(19) United States
(12) Patent Application Publication
(10) Pub. No.: US 2023/0373935 A1
(43) Pub. Date: Nov. 23, 2023

(54) MODULATORS OF CYSTIC FIBROSIS TRANSMEMBRANE CONDUCTANCE REGULATOR

(71) Applicant: VERTEX PHARMACEUTICALS INCORPORATED, Boston, MA (US)

(72) Inventors: Jason MCCARTNEY, Cardiff by the Sea, CA (US); Sunny ABRAHAM, San Diego, CA (US); Corey Don ANDERSON, Brighton, MA (US); Vijayalaksmi ARUMUGAM, San Marcos, CA (US); Jaclyn CHAU, San Diego, CA (US); Thomas CLEVELAND, San Marcos, CA (US); Timothy A. DWIGHT, Quincy, MA (US); Bryan A. FRIEMAN, La Jolla, CA (US); Peter GROOTENHUIS, Del Mar, CA (US); Sara Sabina HADIDA RUAH, La Jolla, CA (US); Yoshihiro ISHIHARA, San Diego, CA (US); Mark Thomas MILLER, Rancho Santa Fe, CA (US); Alina SILINA, Needham, MA (US); Jinglan ZHOU, San Diego, CA (US)

(21) Appl. No.: 18/030,518

(22) PCT Filed: Oct. 6, 2021
(86) PCT No.: PCT/US2021/053863

§ 371 Date: Apr. 6, 2023

Related U.S. Application Data
(60) Provisional application No. 63/089,000, filed on Oct. 7, 2020.

(51) Int. Cl. C07D 239/69; C07D 401/14; C07D 405/14; C07D 403/12; C07D 403/14; C07D 401/12; C07D 403/04; C07D 409/14; C07D 413/04; C07D 401/04; C07D 413/14; C07D 487/04; C07D 471/04; C07D 417/14; C07D 471/08; C07D 405/04; C07D 409/04; C07D 417/04; C07D 413/10; C07F 9/6558; C07D 409/12; C07D 405/12; C07D 239/47; C07D 413/12; C07D 417/12; A61K 45/06

(52) U.S. Cl. CPC C07D 239/69; C07D 401/14; C07D 405/14; C07D 403/12; C07D 403/14; C07D 401/12; C07D 403/04; C07D 409/14; C07D 413/04; C07D 401/04; C07D 413/14; C07D 487/04; C07D 471/04; C07D 417/14; C07D 471/08; C07D 405/04; C07D 409/04; C07D 417/04; C07D 413/10; C07F 9/65583; C07D 409/12; C07D 405/12; C07D 239/47; C07D 413/12; C07D 417/12; A61K 45/06

(57) ABSTRACT

This disclosure provides modulators of Cystic Fibrosis Transmembrane Conductance Regulator (CFTR) having the core structure, pharmaceutical compositions containing at least one such modulator, methods of treating CFTR mediated diseases, including cystic fibrosis using such modulators and pharmaceutical compositions, combination therapies and combination pharmaceuticals employing those modulators, and processes and intermediates for making such modulators.

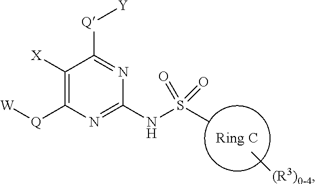

This application claims the benefit of priority of U.S. Provisional Application No. 63/089,000, filed Oct. 7, 2020, the contents of which are incorporated by reference herein in its entirety.

The disclosure relates to modulators of Cystic Fibrosis Transmembrane Conductance Regulator (CFTR), pharmaceutical compositions containing the modulators, methods of treatment of CFTR mediated diseases, including cystic fibrosis, using such modulators and pharmaceutical compositions, combination therapies and combination pharmaceutical compositions employing those modulators, and processes and intermediates for making such modulators.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease-causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 432 of these identified mutations, with sufficient evidence to define 352 mutations as disease-causing. The most prevalent disease-causing mutation is a deletion of phenylalanine at position 508 of the CFTR amino acid sequence and is commonly referred to as the F508del mutation. This mutation occurs in many of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease-causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Na⁺—K⁺-ATPase pump and Cl− channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

A number of CFTR modulating compounds have recently been identified. However, compounds that can treat or reduce the severity of the cystic fibrosis and other CFTR mediated diseases, and particularly the more severe forms of these diseases, are still needed.

One aspect of the invention provides novel compounds, including compounds of Formula I, compounds of Formula Ia, compounds of Formula Ib, compounds of Formula Ic, compounds of Formula Id, Compounds 1-1607 tautomers thereof, deuterated derivatives of those compounds and tautomers, and pharmaceutically acceptable salts of any of the foregoing.

Formula I encompasses compounds falling within the following structure:

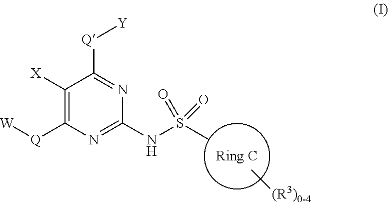

and includes tautomers of those compounds, deuterated derivatives of any of the compounds and tautomers, and pharmaceutically acceptable salts of any of the foregoing, wherein:
Q is absent or is oxygen
W is selected from
—H;
halogen;
—CN;
—C₁₋₈ alkyl;
—C₁₋₈ alkoxy optionally substituted with ═O;
—C₂₋₄ alkenyl;
—C₃₋₄ alkynyl; and
—NH₂ optionally substituted with 1-2 groups selected from C₁₋₆ alkyl;
   or W is

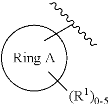

wherein:
Ring A is selected from:
—C₃₋₆ cycloalkyl,
C₅₋₁₀ aryl,
5-10 membered heterocyclyl, and
5-10 membered heteroaryl;
   Each R¹ is independently selected from:
-halogen;
—OH;
—CN;
—C₁₋₆ alkyl optionally substituted with 1-3 groups selected from N(CH₃)₂, OH, ═O, halogen, C₃ cycloalkyl, C₁₋₄ alkoxy, NH₂ (optionally substituted with 1-2 groups independently selected from —C₁₋₄ alkyl, and —C(O)C₁₋₃ alkyl);
—C₁₋₆ alkenyl;
C₁₋₈ alkoxy optionally substituted with 1-3 groups independently selected from halogen, —CN, —OH, ═O, —COOH, —C₃₋₆ cycloalkyl;
—NH₂ optionally substituted with 1-2 groups independently selected from CH₃, —S(O)₂CH₃, —C(O)C₁₋₄ alkyl;
—SR⁴
—S(O)R⁴
—S(O)₂R⁴
   wherein each R⁴ is independently selected from —C₁₋₃ alkyl;
—C₃₋₄ cycloalkyl optionally substituted with C(O)NH₂, C₁₋₃ alkyl;
5-6 membered heterocyclyl optionally substituted with C₁₋₃ alkyl, CF₃;
-phenyl; and
5-6 membered heteroaryl optionally substituted with CH₃;
X is selected from:
hydrogen,
—C₁₋₆ alkyl optionally substituted with 1-5 groups selected from COOH, halogen;
—C₃₋₆ alkenyl;
—C₁₋₆ alkoxy;
—C₃₋₆ cycloalkyl;
—CN;
halogen;
phenyl optionally substituted with 1-2 groups independently selected from halogen, CN, C₁₋₃ alkoxy, C₁₋₃ alkyl; and
—O-phenyl;
Q′ is absent (i.e., Y is attached directly to the pyrimidine core), or is selected from:
—CH₂— optionally substituted with —CN, ═O, or —OH;
—NH— optionally substituted with phenyl; and
—S— optionally substituted with 1-2 ═O;
Y is selected from:
—H;
—C₂₋₄ alkynyl;
—C₁₋₈ alkoxy optionally substituted with 1-3 independently selected from —OH, NHC(O)CH₃;
—C₁₋₈ alkyl optionally substituted with 1-3 independently selected from:
—OH;
—CN,
halogen;
—NH₂ optionally substituted with 1-2 groups independently selected from —C(O)C₁₋₄ alkoxy, —C(O)C₁₋₃ alkyl, —CH₂-phenyl, C₁₋₈ alkyl (optionally further substituted with OH);
—C₃₋₆ cyclic alkyl;
—C₁₋₄ alkoxy
-phenyl optionally substituted with C₁₋₄ alkyl;
—5-6 membered heterocyclyl optionally substituted with 1-2 groups independently selected from ═O, OH;
5-6 membered heteroaryl;
or Y is

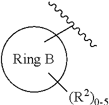

wherein:
Ring B is
—C₅₋₆ aryl;
5-10-membered heteroaryl,
4-10-membered heterocyclyl,
5-10 membered cycloalkyl,
Each R² is independently selected from:
halogen;
—CN;
—OH;
═O;
—C₁₋₈ alkyl optionally substituted with 1-5 groups independently selected from:
   ═O;
   —OH;
   —CN;
   halogen;
   —NH₂ optionally substituted with 1-2 groups independently selected from C₁₋₄ alkyl (optionally substituted with ═O, —CN, —OH, 3-6 membered heterocyclyl (optionally substituted with CH₃), C₁₋₆ alkoxy (optionally substituted with ═O), —C₃₋₆ cyclic alkyl;
   —C₁₋₄ alkoxy;
   —C₃₋₁₀ cyclic alkyl optionally substituted with 1-4 groups selected from ═O, OH, CH₃, CF₃, C₆-cycloalkyl, cyano, NHC(O)C₁₋₆ alkoxy, phenyl (optionally substituted with 1-2 groups selected from halogen, —C₁₋₃ alkyl, C₁₋₃ alkoxy), —C₆ cyclic alkyl);
   —C₆₋₁₀ aryl optionally substituted with 1-3 groups independently selected from halogen, —OH, —C₁₋₄ alkyl (optionally substituted with 1-3 F, —OH, —CN, ═O), —C₁₋₄ alkoxy (optionally substituted with ═O), —CN, —NH₂, S—C₁₋₃ alkyl, 5-6 membered heteroaryl;
   4-10 membered heterocyclyl optionally substituted with 1-3 groups selected from ═O, OH, C₁₋₄ alkyl (optionally substituted with 1-3 F), —C(O)C₁₋₃ alkyl, —C(O)C₁₋₄ alkoxy, phenyl (optionally substituted with CH₃);
   5-10 membered heteroaryl optionally substituted with 1-2 groups selected from ═O, —OH, F, C₁, —C₁₋₄ alkyl (optionally substituted with 1-3 F), C₁₋₄ alkoxy, —C(O)CH₃, phenyl, 5 membered heteroaryl, —NH₂ (optionally substituted with 1-2 groups independently selected from C₁₋₄ alkyl, —C(O)C₁₋₄ alkoxy);
   —SCH₃;
   —S-phenyl optionally substituted with C₁₋₃ alkyl; and
   P(O)(CH₃)₂;
—C₄₋₆ alkynyl optionally substituted with ═O;
—C₁₋₈ alkoxy optionally substituted with 1-3 groups selected from halogen, ═O, NH₂ (optionally substituted with 1-2 groups independently selected from C₁₋₃ alkyl), phenyl (optionally substituted with 1-2 groups selected from halogen ═O, CH₃, —OCH₃, NHC(O)C₁₋₄ alkoxy);
—NH₂ optionally substituted with 1-2 groups independently selected from:
   —C₁₋₆ alkyl optionally substituted with 1-2 groups selected from ═O, —OH, —NH₂, —N(CH₃)₂, —NHCH₃, —S(O)₂CH₃, C₁₋₄ alkoxy, and C₆ cycloalkyl;
   —C₁₋₆ alkoxy optionally substituted with ═O;
   —C₂₋₃ alkynyl;
   5-6 membered heterocyclyl optionally substituted with 1-2 groups independently selected from ═O, —C₁₋₃ alkyl);
   —C₅₋₆ cycloalkyl optionally substituted with COOH;
—S(O)₂ optionally substituted with —C₁₋₃ alkyl (optionally substituted with —N(CH₃)₂),
a cyclic or bridged ring selected from -phenyl, C₃₋₆ cyclic alkyl, 4-8 membered heterocyclyl, and 6 membered heteroaryl, each of which may be optionally substituted with 1-3 groups independently selected from:
   halogen;
   —OH;
   ═O;
   —NH₂ optionally substituted with 1-2 groups independently selected from C(O)C₁₋₄ alkoxy;
   —C₁₋₆ alkyl optionally substituted with a group selected from 1-3 F, —OH, ═O, —NH₂ (optionally substituted with 1-2 groups independently selected from —C₁₋₆ alkoxy);
   —C₁₋₆ alkoxy optionally substituted with ═O; and
   —C₃ cycloalkyl;
Ring C is selected from
—C₃₋₇ cyclic alkyl;
6-membered heterocyclyl;
—C₅₋₆ aryl; and
5-10 membered heteroaryl;
Each R³ is independently selected from
hydrogen
halogen;
—CN;
—OH;
═O;
—C₁₋₆ alkyl optionally substituted with 1-3 groups independently selected from —OH, ═O, halogen, -phenyl, —NH₂ (optionally substituted with 1-2 groups independently selected from —H, —C₁₋₃ alkyl (optionally substituted with C₆ cycloalkyl, phenyl));
—C₁₋₄ alkoxy (optionally substituted with 1-3 groups independently selected from ═O and F),
—NH₂ optionally substituted with 1-2 groups independently selected from ═O, —O⁻, —C(O)CH₃, —CH₃),
6 membered heterocyclyl (optionally substituted with 1-2 F), and
—SO₂CH₃;
   wherein W and Y cannot not both be hydrogen.

Formula I also includes compounds of Formulae Ia, Ib, Ic and Id:

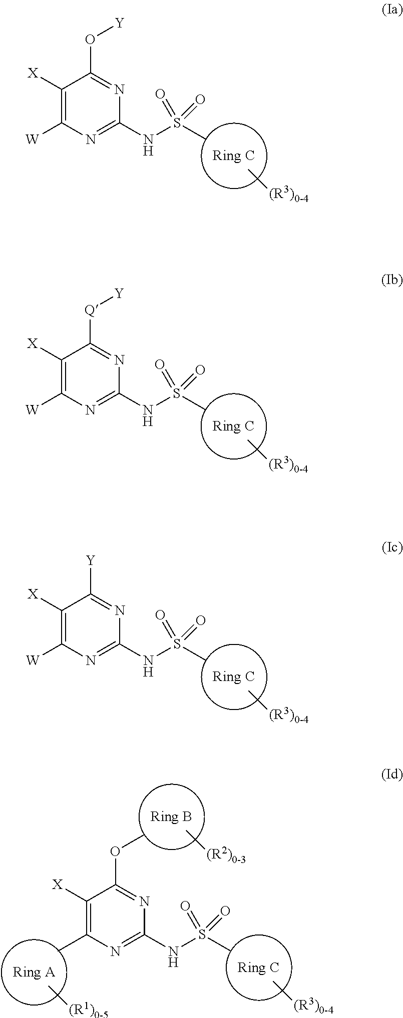

tautomers of those compounds, deuterated derivatives of any of the compounds and tautomers, and pharmaceutically acceptable salts of any of the foregoing, wherein all variables are as defined for Formula I.

Another aspect of the disclosure provides pharmaceutical compositions comprising at least one compound chosen from the novel compounds disclosed herein, pharmaceutically acceptable salts thereof, and deuterated derivatives of any of the foregoing, and at least one pharmaceutically acceptable carrier, which compositions may further include at least one additional active pharmaceutical ingredient. In some embodiments, the at least one additional active pharmaceutical ingredient is at least one other CFTR modulator. In some embodiments, the at least one other CFTR modulator is selected from CFTR potentiators. In some embodiments, the at least one other CFTR modulator is selected from CFTR correctors. In some embodiments, the at least one other CFTR modulators includes both a potentiator and a corrector. In some embodiments, the one or more additional CFTR modulating agents are selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

Thus, another aspect of the invention provides methods of treating the CFTR-mediated disease cystic fibrosis comprising administering at least one of compound chosen from the novel compounds disclosed herein, pharmaceutically acceptable salts thereof, and deuterated derivatives of any of the foregoing, and at least one pharmaceutically acceptable carrier, optionally as part of a pharmaceutical composition comprising at least one additional component, to a subject in need thereof. In some embodiments, the at least one additional active pharmaceutical ingredient is at least one other CFTR modulator. In some embodiments, the at least one other CFTR modulator is selected from CFTR potentiators. In some embodiments, the at least one other CFTR modulator is selected from CFTR correctors. In some embodiments, the at least one other CFTR modulators includes both a potentiator and a corrector. In some embodiments, the one or more additional CFTR modulating agents are selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

In certain embodiments, the pharmaceutical compositions of the disclosure comprise at least one compound chosen from compounds of Formula I, compounds of Formulae Ia-Id, Compounds 1-1607, tautomers thereof, deuterated derivatives of those compounds and tautomers, and pharmaceutically acceptable salts of any of the foregoing. In some embodiments, compositions comprising at least one compound chosen from compounds of Formula I, compounds of Formulae Ia-Id, Compounds 1-1607, tautomers thereof, deuterated derivatives of those compounds and tautomers, and pharmaceutically acceptable salts of any of the foregoing may optionally further comprise (a) at least one compound chosen from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (tezacaftor), 3- (6-(1-(2,2-difluorobenzo[d][1,3]dioxol-5-yl)cyclopropane carboxamido)-3-methylpyridin-2-yl)benzoic acid (lumacaftor), and deuterated derivatives and pharmaceutically acceptable salts thereof; and/or (b) at least one compound chosen from N-[2,4-bis(1,1-dimethylethyl)-5-hydroxyphenyl]-1,4-dihydro-4-oxoquinoline-3-carboxamide (ivacaftor), N-(2-(tert-butyl)-5-hydroxy-4-(2-(methyl-d3)propan-2-yl-1,1,1,3,3,3-d 6)phenyl)-4-oxo-1,4-dihydroquinoline-3-carboxamide (deutivacaftor), (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol , and deuterated derivatives and pharmaceutically acceptable salts of any of the foregoing.

Another aspect of the disclosure provides methods of treating the CFTR-mediated disease, cystic fibrosis, that comprise administering to a patient in need thereof at least one compound chosen from the novel compounds disclosed herein, deuterated derivatives thereof and pharmaceutically acceptable salts of any of the foregoing, and optionally further administering one or more additional CFTR modulating agents. A further aspect of the disclosure provides the pharmaceutical compositions of the disclosure comprising at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing and, optionally, one or more CFTR modulating agents, for use in therapy or for use in the manufacture of a medicament. In some embodiments the optional one or more additional CFTR modulating agents are selected from CFTR potentiators. In some embodiments, the one or more additional CFTR modulating agents are selected from CFTR correctors. In some embodiments, the one or more additional CFTR modulating agents are selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

A further aspect of the disclosure provides intermediates and methods for making the compounds and pharmaceutical compositions disclosed herein.

### DEFINITIONS

“Tezacaftor” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, which can be depicted with the following structure:

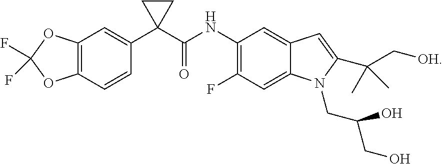 [SMILES: CC(C)(CO)c1cc2cc(NC(=O)C3(c4ccc5c(c4)OC(F)(F)O5)CC3)c(F)cc2n1C[C@@H](O)CO]

Tezacaftor may be in the form of a deuterated derivative, a pharmaceutically acceptable salt, or a pharmaceutically acceptable salt of a deuterated derivative. Tezacaftor and methods of making and using tezacaftor are disclosed in WO 2010/053471, WO 2011/119984, WO 2011/133751, WO 2011/133951, WO 2015/160787, and US 2009/0131492, each incorporated hereing by reference.

“Ivacaftor” as used throughout this disclosure refers to N-(5-hydroxy-2,4-di-tert-butyl-phenyl)-4-oxo-1H-quinoline-3-carboxamide, which is depicted by the structure:

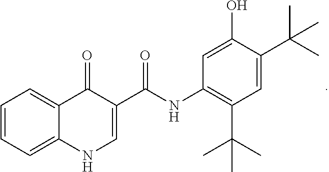 [SMILES: CC(C)(C)c1cc(C(C)(C)C)c(NC(=O)c2c[nH]c3ccccc3c2=O)cc1O]

Ivacaftor may also be in the form of a deuterated derivative, a pharmaceutically acceptable salt, or a pharmaceutically acceptable salt of a deuterated derivative. Ivacaftor and methods of making and using ivacaftor are disclosed in WO 2006/002421, WO 2007/079139, WO 2010/108162, and WO 2010/019239, each incorporated herein by reference.

In some embodiments, a deuterated derivative of ivacaftor (deutivacaftor) is employed in the compositions and methods disclosed herein. A chemical name for deutivacaftor is N-(2-(tert-butyl)-5-hydroxy-4-(2-(methyl-d3)propan-2-yl-1,1,1,3,3,3-d 6)phenyl)-4-oxo-1,4-dihydroquinoline-3-carboxamide, as depicted by the structure:

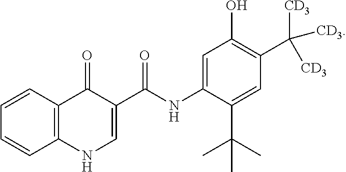 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

Deutivacaftor may be in the form of a further deuterated derivative, a pharmaceutically acceptable salt, or a pharmaceutically acceptable salt of a deuterated derivative. Deutivacaftor and methods of making and using deutivacaftor are disclosed in WO 2012/158885, WO 2014/078842, and U.S. Pat. No. 8,865,902, incorporated herein by reference.

“Lumacaftor” as used herein, refers to 3- (6-(1-(2,2-difluorobenzo[d][1,3]dioxol-5-yl)cyclopropanecarboxamido)-3-methylpyridin-2-yl)benzoic acid, which is depicted by the chemical structure:

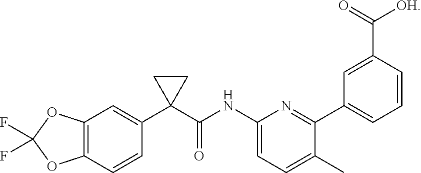 [SMILES: Cc1ccc(NC(=O)C2(c3ccc4c(c3)OC(F)(F)O4)CC2)nc1-c1cccc(C(=O)O)c1]

Lumacaftor may be in the form of a deuterated derivative, a pharmaceutically acceptable salt, or a pharmaceutically acceptable salt of a deuterated derivative. Lumacaftor and methods of making and using Lumacaftor are disclosed in WO 2007/056341, WO 2009/073757, and WO 2009/076142, incorporated herein by reference.

As used herein, the term “alkyl” refers to a saturated or partially saturated, branched or unbranched aliphatic hydrocarbon containing carbon atoms (such as, for example, 1, 2, 3, 4, 5, 6, 7, 8, 9, 10, 11, 12, 13, 14, 15, 16, 17, 18, 19, or 20 carbon atoms) in which one or more adjacent carbon atoms may be interrupted by a double (alkenyl) or triple (akynyl) bond. Alkyl groups may be substituted or unsubstituted.

As used herein, the term “haloalkyl group” refers to an alkyl group substituted with one or more halogen atoms, e.g., fluoroalkyl, wherein the alkyl group is substituted with one or more fluorine atoms.

The term “alkoxy,” as used herein, refers to an alkyl or cycloalkyl covalently bonded to an oxygen atom. Alkoxy groups may be substituted or unsubstituted.

As used herein, “cycloalkyl” refers to a cyclic, bicyclic, tricyclic, or polycyclic non-aromatic hydrocarbon groups having 3 to 12 carbons (such as, for example 3-10 carbons) and may include one or more unsaturated bonds. “Cycloalkyl” groups encompass monocyclic, bicyclic, tricyclic, bridged, fused, and spiro rings, including mono spiro and dispiro rings. Non-limiting examples of cycloalkyl groups are cyclopropyl, cyclobutyl, cyclopentyl, cyclohexyl, adamantyl, norbornyl, dispiro[2.0.2.1]heptane, and spiro[2,3]hexane. Cycloalkyl groups may be substituted or unsubstituted.

The term “aryl,” as used herein, is a functional group or substituent derived from an aromatic ring and encompasses monocyclic, bicyclic, tricyclic, and fused rings. Non-limiting examples of aryl groups include phenyl and naphthyl.

The term “heteroaryl ring,” as used herein, refers to an aromatic ring comprising at least one ring atom that is a heteroatom, such as O, N, or S. Heteroaryl groups encompass monocyclic, bicyclic, tricyclic, bridged, fused, and spiro rings, including mono-spiro and di-spiro rings.

As used herein, the term “heterocyclyl” and “heterocyclyl ring” are used interchangeably and refer to a non-aromatic hydrocarbon containing 3 to 12 atoms in a ring (such as, for example 3-10 atoms) comprising at least one ring atom that is a heteroatom, such as O, N, or S and may include one or more unsaturated bonds. “Heterocyclyl” rings encompass monocyclic, bicyclic, tricyclic, polycyclic, bridged, fused, and spiro rings, including mono spiro and dispiro rings.

“Substituted,” whether preceded by the term “optionally” or not, indicates that at least one hydrogen of the “substituted” group is replaced by a substituent. Unless otherwise indicated, an “optionally substituted” group may have a suitable substituent at each substitutable position of the group, and when more than one position in any given structure may be substituted with more than one substituent chosen from a specified group, the substituent may be either the same or different at each position.

Non-limiting examples of protecting groups for nitrogen include, for example, t-butyl carbamate (Boc), benzyl (Bn), para-methoxybenzyl (PMB), tetrahydropyranyl (THP), 9-fluorenylmethyl carbamate (Fmoc), benzyl carbamate (Cbz), methyl carbamate, ethyl carbamate, 2,2,2-trichloroethyl carbamate (Troc), 2-trimethylsilylethyl carbamate (Teoc), allyl carbamate (Aloc or Alloc), formamide, acetamide, benzamide, allylamine, trifluoroacetamide, triphenylmethylamine, benzylideneamine, and p-toluenesulfonamide. A comprehensive list of nitrogen protecting groups can be found in Wuts, P. G. M. “Greene's Protective Groups in Organic Synthesis: Fifth Edition,” (2014) John Wiley and Sons.

As used herein, “deuterated derivative(s)” refers to a compound having the same chemical structure as a reference compound, with one or more hydrogen atoms replaced by a deuterium atom. In some embodiments, the one or more hydrogens replaced by deuterium are part of an alkyl group. In some embodiments, the one or more hydrogens replaced by deuterium are part of a methyl group.

As used herein, “CFTR” means cystic fibrosis transmembrane conductance regulator.

As used herein, the term “CFTR modulator” refers to a compound that increases the activity of CFTR. The increase in activity resulting from a CFTR modulator includes but is not limited to compounds that correct, potentiate, stabilize and/or amplify CFTR.

The terms “corrector” and “CFTR corrector” are used interchangeably to refer to a compound that facilitates the processing and trafficking of CFTR to increase the amount of CFTR at the cell surface. The novel compounds disclosed herein are CFTR correctors. Other correctors may be used in combination therapies with the novel compounds disclosed herein to treat CFTR mediated diseases, such as cystic fibrosis. Such other correctors include, e.g., tezacaftor, lumacaftor, and their deuterated derivatives and pharmaceutically acceptable salts.

The term “potentiator” and “CFTR potentiator” are used interchangeably to refer to a compound that increases the channel activity of CFTR protein located at the cell surface, resulting in enhanced ion transport. Ivacaftor and deutivacaftor disclosed herein are CFTR potentiators. Potentiators may be used in combination with the novel compounds of the disclosure to treat CFTR mediated diseases such as cystic fibrosis. Such potentiators include, e.g., ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and their deuterated derivatives and pharmaceutically acceptable salts.

It will be appreciated that when a description of a combination of compound selected from compounds of Formula I, compounds of any one of Formulae Ia-Id, Compounds 1-1607, tautomers thereof, deuterated derivatives of those compounds and tautomers, and pharmaceutically acceptable salts of any of the foregoing, and other specified CFTR modulating agents is provided herein, typically, but not necessarily, the combination or treatment regime will include at least one potentiator, such as, e.g., a potentiator selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof. It will also be appreciated that typically, but not necessarily, a single potentiator may used in a combination pharmaceutical composition or therapy. In some embodiments, a combination of at least one compound selected from compounds of Formula I, compounds of any one of Formulae Ia-Id, Compounds 1-1607, tautomers thereof, deuterated derivatives of those compounds and tautomers, and pharmaceutically acceptable salts of any of the foregoing, and other specified CFTR modulating agents, will also include another CFTR corrector, such as, e.g., a corrector compound selected from tezacaftor, lumacaftor, and deuterated derivatives and pharmaceutically acceptable salts thereof.

The term “at least one compound selected from,” as used herein, refers to the selection of one or more of the compounds from a specified group.

A reference to “Compounds 1-1607 in this disclosure is intended to represent a reference to each of Compounds 1 through 1607 individually.

As used herein, the term “active pharmaceutical ingredient” or “therapeutic agent” (“API”) refers to a biologically active compound.

The terms “patient” and “subject” are used interchangeably and refer to an animal including humans.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement in one or more symptoms of CF or lessening the severity of CF or one or more symptoms of CF in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

It should be understood that references herein to methods of treatment (e.g., methods of treating a CFTR mediated disease or a method of treating cystic fibrosis) using one or more compounds of the disclosure optionally in combination with one or more additional CFTR modulating agents (e.g., a compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, optionally in combination with one or more additional CFTR modulating agents) should also be interpreted as references to:
one or more compounds (e.g., compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, optionally in combination with one or more additional CFTR modulating agents) for use in methods of treating, e.g., cystic fibrosis optionally in combination with one or more additional CFTR modulating agents; and/or
the use of one or more compounds (e.g., a compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, optionally in combination with one or more additional CFTR modulating agents) in the manufacture of a medicament for treating, e.g., cystic fibrosis.

It should be also understood that references herein to methods of treatment (e.g., methods of treating a CFTR mediated disease or a method of treating cystic fibrosis) using a pharmaceutical composition of the disclosure (e.g., a pharmaceutical composition comprising at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing and optionally further comprising one or more additional CFTR modulating agents) should also be interpreted as references to:
a pharmaceutical composition (e.g., a pharmaceutical composition comprising at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing and optionally further comprising one or more additional CFTR modulating agents) for use in methods of treating, e.g., cystic fibrosis; and/or
the use of a pharmaceutical composition (e.g., a pharmaceutical composition comprising at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing and optionally further comprising one or more additional CFTR modulating agents) in the manufacture of a medicament for treating, e.g., cystic fibrosis.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

The terms “about” and “approximately” may refer to an acceptable error for a particular value as determined by one of skill in the art, which depends in part on how the values are measured or determined. In some embodiments, the terms “about” and “approximately” mean within 20%, 15%, 10%, 5%, 4%, 3%, 2%, 1%, or 0.5% of a given value or range.

As used herein, the term “solvent” refers to any liquid in which the product is at least partially soluble (solubility of product >1 g/l).

As used herein, the term “room temperature” or “ambient temperature” means 15° C. to 30° C.

It will be appreciated that certain compounds of this invention may exist as separate stereoisomers or enantiomers and/or mixtures of those stereoisomers or enantiomers.

Certain compounds disclosed herein may exist as tautomers and both tautomeric forms are intended, even though only a single tautomeric structure is depicted. For example, a description of Compound X is understood to include its tautomer Compound Y and vice versa, as well as mixtures thereof:

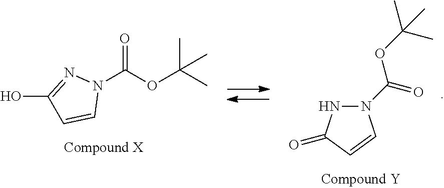

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations;” mutations associated with severe defects in the cellular processing of CFTR and its delivery to the cell surface; mutations associated with no (or minimal) CFTR synthesis; and mutations associated with severe defects in channel conductance.

As used herein, the term “pharmaceutically acceptable salt” refers to a salt form of a compound of this disclosure wherein the salt is nontoxic. Pharmaceutically acceptable salts of the compounds of this disclosure include those derived from suitable inorganic and organic acids and bases. A “free base” form of a compound, for example, does not contain an ionically bonded salt.

The phrase “and deuterated derivatives and pharmaceutically acceptable salts thereof” is used interchangeably with “and deuterated derivatives and pharmaceutically acceptable salts thereof of any of the forgoing” in reference to one or more compounds or formulae of the invention. These phrases are intended to encompass pharmaceutically acceptable salts of any one of the referenced compounds, deuterated derivatives of any one of the referenced compounds, and pharmaceutically acceptable salts of those deuterated derivatives.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

### DETAILED DESCRIPTION OF EMBODIMENTS

In addition to compounds of Formula (I), tautomers thereof, deuterated derivatives of those compounds and tautomers, and pharmaceutically acceptable salts of any of the foregoing, the disclosure provides compounds of Formula I, compounds of Formulae Ia-Id, Compounds 1-1607, tautomers thereof, deuterated derivatives of those compounds and tautomers, and pharmaceutically acceptable salts of any of the foregoing.

For example, in some embodiments, the compound of Formula I is a compound of Formula Ia:

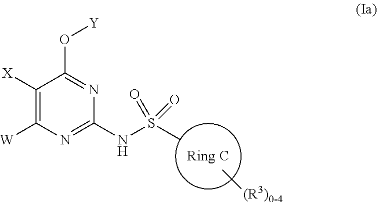

or a tautomer thereof, a deuterated derivative of the compound or tautomer, or a pharmaceutically acceptable salt of any of the foregoing, wherein variables W, X, Y, Ring C, and R³ are as defined for Formula I.

In some embodiments, the compound of Formula I is a compound of Formula Ib:

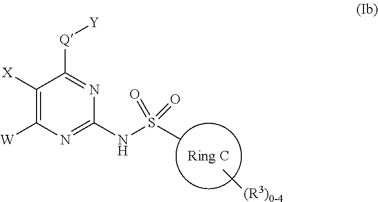

or a tautomer thereof, a deuterated derivative of the compound or tautomer, or a pharmaceutically acceptable salt of any of the foregoing, wherein
Q′ is selected from CH₂ (optionally substituted with —CN, ═O, —OH), NH (optionally substituted with phenyl, CH₃), or S (optionally substituted with 1-2 ═O); and
variables W, X, Y, Ring C, and R³ are as defined for Formula I.

In some embodiments, the compound of Formula I is a compound of Formula Ic:

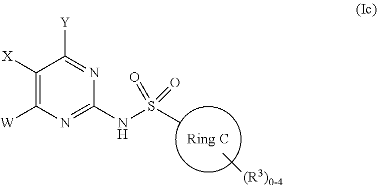

or a tautomer thereof, a deuterated derivative of the compound or tautomer, or a pharmaceutically acceptable salt of any of the foregoing, wherein variables W, X, Y, Ring C, and R³ are as defined for Formula I.

In some embodiments, the compound of Formula I is a compound of Formula Id:

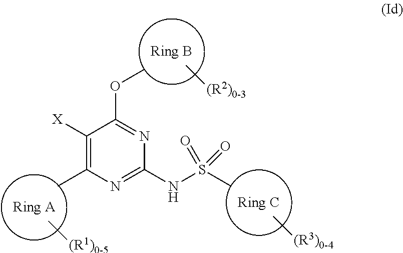

or a tautomer thereof, a deuterated derivative of the compound or tautomer, or a pharmaceutically acceptable salt of any of the foregoing, wherein variables X, R¹, R², and R³, Rings A, B, and C are as defined for Formula I.

Also disclosed herein are Compounds 1-1607 having a structural formula depicted in Table 3, tautomers thereof, deuterated derivatives of those compounds and tautomers, and pharmaceutically acceptable salts of any of the foregoing.

### Methods of Treatment

Any of the novel compounds disclosed herein, such as for example, compounds of Formula I, compounds of Formulae Ia-Id, Compounds 1-1607, tautomers thereof, deuterated derivatives of those compounds and tautomers, and pharmaceutically acceptable salts of any of the foregoing, can act as a CFTR modulator, i.e., it modulates CFTR activity in the body. Individuals suffering from a mutation in the gene encoding CFTR may benefit from receiving a CFTR modulator. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in cystic fibrosis.

Thus, in some embodiments, the invention provides methods of treating, lessening the severity of, or symptomatically treating cystic fibrosis in a patient comprising administering to the patient an effective amount of any of the novel compounds disclosed herein, such as for example, compounds of Formula I, compounds of Formulae Ia-Id, Compounds 1-1607, tautomers thereof, deuterated derivatives of those compounds and tautomers, and pharmaceutically acceptable salts of any of the foregoing, alone or in combination with another active ingredient, such as one or more CFTR modulating agents. In some embodiments, the one (or more) CFTR modulating agent is a corrector. In some embodiments, the one (or more) CFTR modulating agent is a potentiator. In some embodiments, the CFTR modulating agents include both a corrector and a potentiator. In some embodiments, the one or more CFTR modulating agents are selected from potentiators: ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing; and correctors: lumacaftor, tezacaftor, and deuterated derivatives and pharmaceutically acceptable salts thereof.

In some embodiments, the patient to be treated has an F508del/minimal function (MF) genotype, F508del/F508del genotype (homozygous for the F508del mutation), F508del/gating genotype, or F508del/residual function (RF) genotype. In some embodiments the patient is heterozygous and has one F508del mutation. In some embodiments the patient is homozygous for the N1303K mutation.

In some embodiments, 5 mg to 500 mg of a compound disclosed herein, a tautomer thereof, a deuterated derivatives of the compound and tautomer, or a pharmaceutically acceptable salt of any of the foregoing are administered daily.

In some embodiments, the patient has at least one F508del mutation in the CFTR gene. In some embodiments, the patient has a CFTR gene mutation that is responsive to a compound, tautomer, deutrated derivative, or pharmaceutically acceptable salt of the invention based on in vitro data. In some embodiments, the patient is heterozygous and has an F508del mutation on one allele and a mutation on the other allele selected from Table 2:

TABLE 2
CFTR Mutations

| MF Mutation | | | | | |
|---|---|---|---|---|---|
| Category | Mutation | | | | |
| Nonsense | Q2X | L218X | Q525X | R792X | E1104X |
| mutations | S4X | Q220X | G542X | E822X | W1145X |
| | W19X | Y275X | G550X | W882X | R1158X |
| | G27X | C276X | Q552X | W846X | R1162X |
| | Q39X | Q290X | R553X | Y849X | S1196X |
| | W57X | G330X | E585X | R851X | W1204X |
| | E60X | W401X | G673X | Q890X | L1254X |
| | R75X | Q414X | Q685X | S912X | S1255X |
| | L88X | S434X | R709X | Y913X | W1282X |
| | E92X | S466X | K710X | Q1042X | Q1313X |
| | Q98X | S489X | Q715X | W1089X | Q1330X |
| | Y122X | Q493X | L732X | Y1092X | E1371X |
| | E193X | W496X | R764X | W1098X | Q1382X |
| | W216X | C524X | R785X | R1102X | Q1411X |
| Canonical | 185 + 1G→T | 711 + 5G→A | 1717 − 8G→A | 2622 + 1G→A | 3121 − 1G→A |
| splice | 296 + 1G→A | 712 − 1G→T | 1717 − 1G→A | 2790 − 1G→C | 3500 − 2A→G |
| mutations | 296 + 1G→T | 1248 + 1G→A | 1811 + 1G→C | 3040G→C | 3600 + 2insT |
| | 405 + 1G→A | 1249 − 1G→A | 1811 + 1.6 kbA→G | (G970R) | 3850 − 1G→A |
| | 405 + 3A→C | 1341 + 1G→A | 1811 + 1643G→T | 3120G→A | 4005 + 1G→A |
| | 406 − 1G→A | 1525 − 2A→G | 1812 − 1G→A | 3120 + 1G→A | 4374 + 1G→T |
| | 621 + 1G→T | 1525 − 1G→A | 1898 + 1G→A | 3121 − 2A→G | |
| | 711 + 1G→T | | 1898 + 1G→C | | |
| Small (≤3 | 182delT | 1078delT | 1677delTA | 2711delT | 3737delA |
| nucleotide) | 306insA | 1119delA | 1782delA | 2732insA | 3791delC |
| insertion/deletion | 306delTAGA | 1138insG | 1824delA | 2869insG | 3821delT |
| (ins/del) | 365-366insT | 1154insTC | 1833delT | 2896insAG | 3876delA |
| frameshift | 394delTT | 1161delC | 2043delG | 2942insT | 3878delG |
| mutations | 442delA | 1213delT | 2143delT | 2957delT | 3905insT |
| | 444delA | 1259insA | 2183AA→G^{a} | 3007delG | 4016insT |
| | 457TAT→G | 1288insTA | 2184delA | 3028delA | 4021dupT |
| | 541delC | 1343delG | 2184insA | 3171delC | 4022insT |
| | 574delA | 1471delA | 2307insA | 3171insC | 4040delA |
| | 663delT | 1497delGG | 2347delG | 3271delGG | 4279insA |
| | 849delG | 1548delG | 2585delT | 3349insT | 4326delTC |
| | 935delA | 1609del CA | 2594delGT | 3659delC | |
| Non-small (>3 | CFTRdele1 | | CFTRdele 16-17b | 1461ins4 | |
| nucleotide) | CFTRdele2 | | CFTRdele17a, 17b | 1924del7 | |
| insertion/deletion | CFTRdele2, 3 | | CFTRdele17a-18 | 2055del9→A | |
| (ins/del) frameshift | CFTRdele2-4 | | CFTRdele19 | 2105- | |
| mutations | CFTRdele3-10, 14b-16 | | CFTRdele19-21 | 2117del13insAGAAA | |
| | CFTRdele4-7 | | CFTRdele21 | 2372del8 | |
| | CFTRdele4-11 | | CFTRdele22-24 | 2721del11 | |
| | CFTR50 kbdel | | CFTRdele22, 23 | 2991del32 | |
| | CFTRdup6b-10 | | 124del23bp | 3667ins4 | |
| | CFTRdele11 | | 602del14 | 4010del4 | |
| | CFTRdele13, 14a | | 852del22 | 4209TGTT→AA | |
| | CFTRdele14b-17b | | 991del5 | | |
| Missense | A46D | V520F | Y569D | N1303K | |
| mutations that | G85E | A559T | L1065P | | |
| Are not | R347P | R560T | R1066C | | |
| responsive in | L467P | R560S | L1077P | | |
| vitro to TEZ, | I507del | A561E | M1101K | | |
| IVA, or | | | | | |
| TEZ/IVA | | | | | |
| and | | | | | |
| % PI >50% and | | | | | |
| SwCl⁻ >86 | | | | | |
| mmol/L | | | | | |

^{a}Also known as 2183delAA→G.
CFTR: cystic fibrosis transmembrane conductance regulator;
IVA: ivacaftor;
SwCl: sweat chloride;
TEZ: tezacaftor
Source: CFTR2.org [Internet]. Baltimore (MD): Clinical and functional translation of CFTR. The Clinical and Functional Translation of CFTR (CFTR2), US Cystic Fibrosis Foundation, Johns Hopkins University, the Hospital for Sick Children. Available at: http://www.cftr2.org/. Accessed 15 May 2018.
Notes:
% PI: percentage of F508del-CFTR heterozygous patients in the CFTR2 patient registry who are pancreatic insufficient; SwCl: mean sweat chloride of F508del-CFTR heterozygous patients in the CFTR2 patient registry.

In some embodiments, the disclosure also is directed to methods of treatment using isotope-labelled compounds of the afore-mentioned compounds, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P, ³⁵S, ¹⁸F and ³⁶Cl, respectively.

The isotope-labelled compounds and salts can be used in a number of beneficial ways. They can be suitable for medicaments and/or various types of assays, such as substrate tissue distribution assays. For example, tritium (³H)— and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (²H)-labelled ones. In some specific embodiments, the isotope-labelled compounds and salts are deuterium (²H)-labelled, wherein one or more hydrogen atoms therein have been replaced by deuterium. In chemical structures, deuterium is represented as “D.”

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Combination Therapies

One aspect disclosed herein provides methods of treating cystic fibrosis and other CFTR mediated diseases using any of the novel compounds disclosed herein, such as for example, compounds of Formula I, compounds of any one of Formulae Ia-Id, Compounds 1-1607, tautomers thereof, deuterated derivatives of those compounds and tautomers, and pharmaceutically acceptable salts of any of the foregoing, in combination with at least one additional active pharmaceutical ingredient.

In some embodiments, at least one additional active pharmaceutical ingredient is selected from mucolytic agents, bronchodilators, antibiotics, anti-infective agents, and anti-inflammatory agents.

In some embodiments, the additional therapeutic agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In some embodiments, the additional agent is a mucolyte. Exemplary mucolytes useful herein includes Pulmozyme®.

In some embodiments, the additional agent is a bronchodilator. Exemplary bronchodiltors include albuterol, metaprotenerol sulfate, pirbuterol acetate, salmeterol, or tetrabuline sulfate.

In some embodiments, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simavastatin.

In some embodiments, the additional agent is a nutritional agent. Exemplary nutritional agents include pancrelipase (pancreating enzyme replacement), including Pancrease®, Pancreacarb®, Ultrase®, or Creon®, Liprotomase® (formerly Trizytek®), Aquadeks®, or glutathione inhalation. In one embodiment, the additional nutritional agent is pancrelipase.

In some embodiments, at least one additional active pharmaceutical ingredient is selected from CFTR modulating agents. In some embodiments, the additional active pharmaceutical ingredient is selected from CFTR potentiators. In some embodiments, the potentiator is selected from ivacaftor, deutivacaftor, and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. In some embodiments, the additional active pharmaceutical ingredient is chosen from CFTR correctors. In some embodiments, the correctors are selected from lumacaftor, tezacaftor, deuterated derivatives of lumacaftor and tezacaftor, and pharmaceutically acceptable salts of any of the foregoing. In some embodiments, the additional active pharmaceutical ingredient includes both a CFTR potentiator and a CFTR corrector.

In some embodiments, the at least one additional active pharmaceutical ingredient is chosen from (a) tezacaftor, lumacaftor, and deuterated derivatives and pharmaceutically acceptable salts thereof; and/or (b) ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and pharmaceutically acceptable salts of any of the foregoing. Thus, in some embodiments, the combination therapies provided herein comprise (a) a compound selected from compounds of Formula I, compounds of any one of Formulae Ia-Id, Compounds 1-1607, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof; or (c) at least one compound selected from ivacaftor, deutivacaftor, and deuterated derivatives and pharmaceutically acceptable salts thereof. In some embodiments, the combination therapies provided herein comprise (a) at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof; and (c) at least one compound selected from ivacaftor, deutivacaftor, and deuterated derivatives and pharmaceutically acceptable salts thereof. In some embodiments, the combination therapies provided herein comprise (a) at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof; and (c) at least one compound selected from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof. some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in combination with at least one compound chosen from ivacaftor and deuterated derivatives and pharmaceutically acceptable salts thereof. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in combination with at least one compound chosen from deutivacaftor and deuterated derivatives and pharmaceutically acceptable salts thereof. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in combination with at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in combination with at least one compound chosed from tezacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in combination with at least one compound chosed from lumacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

Each of the compounds of Formula I, compounds of any one of Formulae Ia-Id, Compounds 1-1607, tautomers thereof, deuterated derivatives of those compounds and tautomers, and pharmaceutically acceptable salts of any of the foregoing, independently can be administered once daily, twice daily, or three times daily. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered once daily. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered twice daily.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered once daily. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered twice daily.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, and at least one compound chosen from lumcafter and deuterated derivatives and pharmaceutically acceptable salts thereof are administered once daily. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, and at least one compound chosen from lumacaftor and deuterated derivatives and pharmaceutically acceptable salts thereof are administered twice daily.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, and at least one compound chosen from ivacaftor, deutivacaftor, and deuterated derivatives and pharmaceutically acceptable salts thereof are administered once daily. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, and at least one compound chosen from ivacaftor, deutivacaftor, and deuterated derivatives and pharmaceutically acceptable salts thereof are administered twice daily.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof are administered once daily. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof are administered twice daily.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from ivacaftor deutivacaftor, and deuterated derivatives and pharmaceutically acceptable salts thereof are administered once daily. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from ivacaftor, deutivacaftor and deuterated derivatives and pharmaceutically acceptable salts thereof are administered twice daily.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, at least one compound chosen from ivacaftor, deutivacaftor, and deuterated derivatives and pharmaceutically acceptable salts thereof, and at least one compound chosen from lumacaftor and pharmaceutically acceptable salts thereof, are administered once daily. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, at least one compound chosen from ivacaftor, deutivacaftor, and deuterated derivatives and pharmaceutically acceptable salts thereof, and at least one compound chosen from lumacaftor and deuterated derivatives and pharmaceutically acceptable salts thereof, are administered twice daily.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, at least one compound chosen from tezacaftor, lumacaftor, and deuterated derivatives and pharmaceutically acceptable salts thereof, and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, are administered once or twice daily.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof, are administered once daily and at least one compound chosen from ivacaftor and deuterated derivatives and pharmaceutically acceptable salts thereof, are administered twice daily. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, and at least one compound chosen from lumacaftor and deuterated derivatives and pharmaceutically acceptable salts thereof, are administered once daily and at least one compound chosen from ivacaftor and pharmaceutically acceptable salts thereof, are administered twice daily.

Compounds of Formula I, compounds of any one of Formulae Ia-Id, Compounds 1-1607, tautomers thereof, deuterated derivatives of those compounds and tautomers, and pharmaceutically acceptable salts of any of the foregoing, tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof can be administered in a single pharmaceutical composition or separate pharmaceutical compositions. Such pharmaceutical compositions can be administered once daily or multiple times daily, such as twice daily. As used herein, the phrase that a given amount of API (e.g., tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, or a deuterated derivative or pharmaceutically acceptable salt thereof) “is administered once or twice daily or per day” means that said given amount is administered per dosing once or twice daily.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; and at least one compound chosen from ivacaftor and deuterated derivatives and pharmaceutically acceptable salts thereof is administered in a third pharmaceutical composition.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; at least one compound chosen from deutivacaftor and deuterated derivatives and pharmaceutically acceptable salts thereof is administered in a third pharmaceutical composition.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof is administered in a third pharmaceutical composition.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in a first pharmaceutical composition; at least one compound chosen from ivacftor, deutivacaftor, and deuterated derivatives and pharmaceutically acceptable salts thereof is administered in a second pharmaceutical composition; at least one compound chosen from lumacaftor and deuterated derivatives and pharmaceutically acceptable salts thereof is administered in a third pharmaceutical composition. In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in a first pharmaceutical composition; at least one compound chosen from lumacaftor, and deuterated derivatives and pharmaceutically acceptable salts thereof is administered in a second pharmaceutical composition; at least one compound chosen from (6R,12R)-17-amino-12 -methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof is administered in a third pharmaceutical composition.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, is administered in a first pharmaceutical composition; and at least one compound chosen from tezacaftor and deuterated derivatives and pharmaceutically acceptable salts thereof and at least one compound chosen from ivacaftor, deutivacaftor, and deuterated derivatives and pharmaceutically acceptable salts thereof are administered in a second pharmaceutical composition. In some embodiments, the second pharmaceutical composition comprises a half of a daily dose of ivacaftor, and the other half dose of ivacaftor hereof is administered in a third pharmaceutical composition.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing; at least one compound chosen from tezacaftor and deuterated derivatives and pharmaceutically acceptable salts thereof and at least one compound chosen from ivacaftor, deutivacaftor, and deuterated derivatives and pharmaceutically acceptable salts thereof are administered in a first pharmaceutical composition. In some embodiments, the first pharmaceutical composition is administered to the patient twice daily. In some embodiments the first pharmaceutical composition is administered once daily. In some embodiments the first pharmaceutical composition is administered once daily and a second composition comprising only ivacaftor is administered once daily.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing; at least one compound chosen from lumacaftor and deuterated derivatives and pharmaceutically acceptable salts thereof and at least one compound chosen from ivacaftor, deutivacaftor, and deuterated derivatives and pharmaceutically acceptable salts thereof are administered in a first pharmaceutical composition. In some embodiments, the first pharmaceutical composition is administered to the patient twice daily. In some embodiments the first pharmaceutical composition is administered once daily. In some embodiments the first pharmaceutical composition is administered once daily and a second composition comprising only ivacaftor is administered once daily.

In some embodiments, at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing; at least one compound chosen from tezacaftor, lumacaftor, and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof are administered in a first pharmaceutical composition. In some embodiments, the first pharmaceutical composition is administered to the patient twice daily. In some embodiments the first pharmaceutical composition is administered once daily.

Any suitable pharmaceutical compositions can be used for compounds of Formula I, compounds of any one of Formulae Ia-Id, Compounds 1-1607, tezacaftor, ivacaftor, deutivacaftor, lumacaftor and tautomers thereof, deuterated derivatives of those compounds and tautomers, and pharmaceutically acceptable salts of any of the foregoing. Some exemplary pharmaceutical compositions for tezacaftor and its deuterated derivatives and pharmaceutically acceptable salts can be found in WO 2011/119984 and WO 2014/014841, wach of which is incorporated herein by reference. Some exemplary pharmaceutical compositions for ivacaftor and its deuterated derivatives and pharmaceutically acceptable salts can be found in WO 2007/134279, WO 2010/019239, WO 2011/019413, WO 2012/027731, and WO 2013/130669, and some exemplary pharmaceutical compositions for deutivacaftor and its deuterated derivatives and pharmaceutically acceptable salts can be found in U.S. Pat. Nos. 8,865,902, 9,181,192, 9,512,079, WO 2017/053455, and WO 2018/080591, all of which are incorporated herein by reference. Some exemplary pharmaceutical compositions for lumacaftor and its deuterated derivatives and pharmaceutically acceptable salts can be found in WO 2010/037066, WO 2011/127421, and WO 2014/071122, incorporated herein by reference.

### Pharmaceutical Compositions

Another aspect of the disclosure provides a pharmaceutical composition comprising at least one compound chosen from compounds of Formula I, compounds of any one of Formulae Ia-Id, Compounds 1-1607, tautomers thereof, deuterated derivatives of those compounds and tautomers, and pharmaceutically acceptable salts of any of the foregoing, and at least one pharmaceutically acceptable carrier.

In some embodiments, the disclosure provides pharmaceutical compositions comprising at least one compound chosen from compounds of Formula I, compounds of Formulae Ia-Id, Compounds 1-1607, tautomers thereof, deuterated derivatives of those compounds and tautomers, and pharmaceutically acceptable salts of any of the foregoing, in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises at least one compound chosen from compounds of Formula I, compounds of Formulae Ia-Id, Compounds 1-1607, tautomers thereof, deuterated derivatives of those compounds and tautomers, and pharmaceutically acceptable salts of any of the foregoing, and at least two additional active pharmaceutical ingredients, one of which is a CFTR corrector and one of which is a CFTR potentiator.

In some embodiments, the disclosure provides a pharmaceutical composition comprising (a) at least one compound chosen from compounds of Formula I, compounds of Formulae Ia-Id, Compounds 1-1607, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of Formulae Ia-Id, Compounds 1-1607, tautomers thereof, deuterated derivatives of those compounds and tautomers, and pharmaceutically acceptable salts of any of the foregoing, (b) at least one compound chosen from ivacaftor, deutivacaftor, and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of Formulae Ia-Id, Compounds 1-1607, tautomers thereof, deuterated derivatives of those compounds and tautomers, and pharmaceutically acceptable salts of any of the foregoing, (b) at least one compound chosen from lumacaftor and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of Formulae Ia-Id, Compounds 1-1607, tautomers thereof, deuterated derivatives of those compounds and tautomers, and pharmaceutically acceptable salts of any of the foregoing, (b) at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of Formulae Ia-Id, Compounds 1-1607, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from ivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of Formulae Ia-Id, Compounds 1-1607, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from deutivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of Formulae Ia-Id, Compounds 1-1607, tautomers thereof, deuterated derivatives of those compounds and tautomers, and pharmaceutically acceptable salts of any of the foregoing, (b) at least one compound chosen from ivacaftor, deutivacaftor, and deuterated derivatives and pharmaceutically acceptable salts thereof, (c) at least one compound chosen from lumacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of Formulae Ia-Id, Compounds 1-1607, tautomers thereof, deuterated derivatives of those compounds and tautomers, and pharmaceutically acceptable salts of any of the foregoing, (b) at least one compound chosen from tezacaftor, lumacaftor, and deuterated derivatives and pharmaceutically acceptable salts thereof, (c) at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, and (d) at least one pharmaceutically acceptable carrier.

Any pharmaceutical composition disclosed herein may comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, lubricants.

The pharmaceutical compositions described herein are useful for treating cystic fibrosis and other CFTR mediated diseases.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21^{st} edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### EXAMPLES

### I. Abbreviation List

ACN: Acetonitrile
Boc anhydride ((Boc)₂₀): Di-tert-butyl decarbonate
CDCl₃: Chloroform-d CDI: Carbonyl diimidazole
CDMT: 2-Chloro-4,6-dimethoxy-1,3,5-triazine
CH₂Cl₂: Dichloromethane
CH₃CN: Acetonitrile
COMU: (1-Cyano-2-ethox-2-oxoethylidenaminooxy)dimethylamino-morpholino-carbenium
hexafluorophosphate
Cmpd: Compound
DABCO: 1,4-Diazabicyclo[2.2.2]octane
DBU: 1,8-Diazabicyclo(5.4.0)undec-7-ene
DCE: 1,2-Dichloroethane
DCM: Dichloromethane
DI: Deionized
DIAD: Diisopropyl azodicarboxylate
DIEA: (DIPEA, DiPEA): N,N-diisopropylethylamine
DMA: N,N-Dimethylacetamide
DMAP: 4-Dimethylaminopyridine
DMF: N,N-Dimethylformamide
DMSO: Dimethyl sulfoxide
DMP: Dess-Martin periodinane
EA: Ethyl acetate
EDC: 1-Ethyl-3-(3-dimethylaminopropyl)carbodiimide
ELSD: Evaporative light scattering detector
ESI-MS: Electrospray ionization mass spectrometry
diethylether: Diethyl ether
EtOAc: Ethyl acetate
EtOH: Ethanol
GC: Gas chromatography
Grubbs 1st Generation catalyst: Dichloro(benzylidene)bis(tricyclohexylphosphine)ruthenium(II)
Grubbs 2^{nd} Generation catalyst: [1,3-Bis(2,4,6-trimethylphenyl)imidazolidin-2-ylidene]-dichloro-[(2-isopropoxyphenyl)methylene]ruthenium
HATU: 1-[Bis(dimethylamino)methylene]-1H-1,2,3-triazolo[4,5-b]pyridinium 3-oxid hexafluorophosphate
HPLC: High-performance liquid chromatography
Hoveyda-Grubbs 2^{nd} Generation catalyst: (1,3-LCis-(2,4,6-trimethylphenyl)-2-imidazolidinylidene)dichloro(o-isopropoxyphenylmethylene)ruthenium, Dichloro[1,3-bis(2,4,6-trimethylphenyl)-2-imidazolidinylidene](2-isopropoxyphenylmethylene)ruthenium(II)
IPA: Isopropanol
KHSO₄: Potassium bisulfate
LC: Liquid chromatography
LCMS: Liquid chromatography mass spectrometry
LCMS Met.: LCMS method
LCMS Rt: LCMS retention time
LDA: Lithium diisopropylamide
LiOH: Lithium hydroxide
MeCN: Acetonitrile
MeOH: Methanol
MeTHF or 2-MeTHF: 2-Methyltetrahydrofuran
MgSO₄: Magnesium sulfate
MTBE: Methyl tert-butyl ether
NaHCO₃: Sodium bicarbonate
NaOH: Sodium hydroxide
NMP: N-Methyl-2-pyrrolidone
NMM: N-Methylmorpholine
Pd/C: Palladium on carbon
Pd₂(dba)₃: Tris(dibenzylideneacetone)dipalladium(O)
Pd(dppf)Cl₂: [1,1′-Bis(diphenylphosphino)ferrocene]dichloropalladium(II)
Pd(OAc)₂: Palladium(II) acetate
PTFE: Polytetrafluoroethylene
rt, RT: Room temperature
RuPhos: 2-Dicyclohexylphosphino-2′, 6′-diisopropoxybiphenyl
SFC: Supercritical fluid chromatography
TBAI: Tetrabutylammonium iodide
TEA: Triethylamine
TFA: Trifluoroacetic acid
THF: Tetrahydrofuran
TLC: Thin layer chromatography
TMS: Trimethylsilyl
TMSCl: Trimethylsilyl chloride
UPLC: Ultra performance liquid chromatography
XANTPHOS: 4,5-Bis(diphenylphosphino)-9,9-dimethylxanthene

### II. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution, respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. 19F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before fourrier transform. 19F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with an ID PFG, 5 mm, 50-202/500 MHz probe (model/part #99337300).

Final purity of compounds was determined by reversed phase UPLC using an Acquity UPLC BEH C₁₈ column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range. Optical purity of methyl (2S)-2,4-dimethyl-4-nitro-pentanoate was determined using chiral gas chromatography (GC) analysis on an Agilent 7890A/MSD 5975C instrument, using a Restek Rt-βDEXcst (30 m×0.25 mm×0.25 μm_df) column, with a 2.0 mL/min flow rate (H2 carrier gas), at an injection temperature of 220° C. and an oven temperature of 120° C., 15 minutes.

### II. General UPLC/HPLC Analytical Methods

LC method A: Analytical reverse phase UPLC using an Acquity UPLC BEH C₁₈ column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B═CH₃CN (0.035% CF₃CO₂H). Flow rate=1.2 mL/minutes, injection volume=1.5 μL, and column temperature=60° C.

LC method B: Reverse phase HPLC using a Kinetex C₁₈ column (50×3.0 mm) and a dual gradient run from 5-100% mobile phase B over 6 minutes. Mobile phase A=H₂O (0.1% CF₃CO₂H). Mobile phase B═CH₃CN (0.1% CF₃CO₂H). Flow rate=1.5 mL/minutes, injection volume=2 μL, and column temperature=60° C.

LC method C: Kinetex C₁₈ 4.6 ×50 mm 2.6 μm. Temp: 45° C., Flow: 2.0 mL/min, Run Time: 3 minutes. Mobile phase: Initial 95% water (0.1% formic acid) and 5% acetonitrile (0.1% formic acid) linear gradient to 95% acetonitrile (0.1% formic acid) for 2.0 minutes then hold at 95% acetonitrile (0.1% formic acid) for 1.0 minutes.

LC method D: Acquity UPLC BEH C₁₈ column (30×2.1 mm, 1.7 m particle) made by Waters (pn: 186002349), and a dual gradient run from 1-99% mobile phase B over 1.0 minute. Mobile phase A=H₂O (0.05% CF₃CO₂H). Mobile phase B═CH₃CN (0.035% CF₃CO₂H). Flow rate=1.5 mL/minute, injection volume=1.5 μL, and column temperature=60° C.

LC method E: Analytical reverse phase UPLC using an Acquity UPLC BEH C₁₈ column (50×2.1 mm, 1.7 m particle) made by Waters (pn: 186002350), and a dual gradient run from 1-99% mobile phase B over 2.5 minutes. Mobile phase A=water (0.05% trifluoroacetic acid). Mobile phase B=acetonitrile (0.035% trifluoroacetic acid). Flow rate=1.2 mL/minute, injection volume=1.5 μL, and column temperature=60° C.

LC method F: UPLC: Reverse phase HPLC using a Kinetex C₁₈ column (50×2.1 mm, 1.7 μm particle) from Phenomenex (pn: 00B-4475-AN)), and a dual gradient run from 1-99% mobile phase B over 2.5 minutes. Mobile phase A=H₂O (0.05% CF₃CO₂H). Mobile phase B═CH₃CN (0.05% CF₃CO₂H). Flow rate=2 mL/min, injection volume=3 μL, and column temperature=50° C.

LC method G: Symmetry, 4.6×75 mm 3.5 μm. Temp: 45° C., Flow: 2.0 mL/min, Run Time: 8 minutes. Mobile Phase: Initial 95% H₂O (0.1% Formic Acid) and 5% CH₃CN (0.1% FA) linear gradient to 95% CH₃CN (0.1% formic acid) for 6.0 minutes then hold at 95% CH₃CN (0.1% formic acid) for 2.0 minutes.

LC method H: Kinetex C₁₈ 4.6 ×50 mm 2.6 um. Temp: 45° C., Flow: 2.0 mL/min, Run Time: 6 minutes. Mobile Phase: Initial 95% H₂O (0.1% Formic Acid) and 5% CH₃CN (0.1% FA) linear gradient to 95% CH₃CN (0.1% FA) for 4.0 minutes then hold at 95% CH₃CN (0.1% FA) for 2.0 minutes.

LC method I: Acquity UPLC BEH C₁₈ column (50×2.1 mm, 1.7 m particle) made by Waters (pn:186002350), and a dual gradient run from 1-99% mobile phase B over 5.0 minutes. Mobile phase A=H₂O (0.05% CF₃CO₂H). Mobile phase B═CH₃CN (0.035% CF₃CO₂H). Flow rate=1.2 mL/minute, injection volume=1.5 μL, and column temperature=60° C.

LC method J: Reverse phase UPLC using an Acquity UPLC BEH C₁₈ column (50×2.1 mm, 1.7 m particle) made by Waters (pn: 186002350), and a dual gradient run from 1-99% mobile phase B over 2.9 minutes. Mobile phase A=H₂O (0.05% NH₄HCO2). Mobile phase B═CH₃CN. Flow rate=1.2 mL/minute, injection volume=1.5 μL, and column temperature=60° C.

LC method K: Kinetex Polar C₁₈ 3.0 ×50 mm 2.6 μm, 3 min, 5-95% ACN in H₂O (0.1% Formic Acid) 1.2 mL/minute.

LC method L: Reverse phase UPLC using an Acquity UPLC BEH C₁₈ column (100 ×2.1 mm, 1.7 m particle) made by Waters (pn: 186002352), and a dual gradient run from 1-99% mobile phase B over 14.0 minutes. Mobile phase A=H₂O (0.05% CF₃CO₂H). Mobile phase B═CH₃CN (0.035% CF₃CO₂H). Flow rate=0.8 mL/minute, injection volume=1.5 μL, and column temperature=60° C.

LC method M: Poroshell 120 EC-Cis 3.0×50 mm 2.7 μM, Temp: 45° C., Flow: 2.0 ml/min, Run Time: 6 minutes. Mobile Phase Conditions: Initial 95% H₂O (0.1% FA) and 5% CH₃CN (0.1% FA) linear gradient to 95% CH₃CN (0.1% FA) for 4.0 min then hold at 95% CH₃CN (0.1% FA) for 2.0 minutes.

LC method N: Kinetex EVO C₁₈ 4.6 ×50 mm 2.6 m, Temp: 45° C., Flow: 2.0 mL/min, Run Time: 4 minutes. Mobile Phase: Initial 95% H₂O (0.1% Formic Acid) and 5% CH₃CN (0.1% FA) linear gradient to 95% CH₃CN (0.1% FA) for 2.0 min then hold at 95% CH₃CN (0.1% FA) for 2.0 minutes.

LC method O: Zorbax C₁₈ 4.6 ×50 mm 3.5 μM, 2.0 mL/minute, 95% H₂O (0.1% formic acid)+5% CH₃CN (0.1% FA) to 95% CH₃CN (0.1% FA) gradient (2.0 min) then hold at 95% CH₃CN (0.1% FA) for 1.0 minute.

LC method P: Poroshell 120 EC-C₁₈ 3.0 ×50 mm 2.7 μM, Temp:45° C., Flow: 1.5 mL/minute, Run Time: 3 mins. Mobile phase conditions: Initial. 95% H₂O (0.1% Formic Acid) and 5% CH₃CN (0.1% FA) linear gradient to 95% CH₃CN (0.1% FA) for 1.5 minute then hold at 95% CH₃CN (0.1% FA) for 1.5 minutes.

LC method Q: Reversed phase UPLC using an Acquity UPLC BEH C₁₈ column (50 ×2.1 mm, 1.7 m particle) made by Waters (pn: 186002350), and a dual gradient run from 30-99% mobile phase B over 2.9 minutes. Mobile phase A=H₂O (0.05% CF₃CO₂H). Mobile phase B═CH₃CN (0.035% CF₃CO₂H). Flow rate=1.2 mL/minutes, injection volume=1.5 μL, and column temperature=60° C.

LC method R: Reversed phase UPLC using an Acquity UPLC BEH C₁₈ column (50 ×2.1 mm, 1.7 m particle) made by Waters (pn: 186002350), and a dual gradient run from 1-99% mobile phase B over 1.9 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### IV. Synthesis of Common Intermediates

### A. Preparation of 4-Chloro-6-(2,6-dimethylphenyl)pyrimidin-2-amine

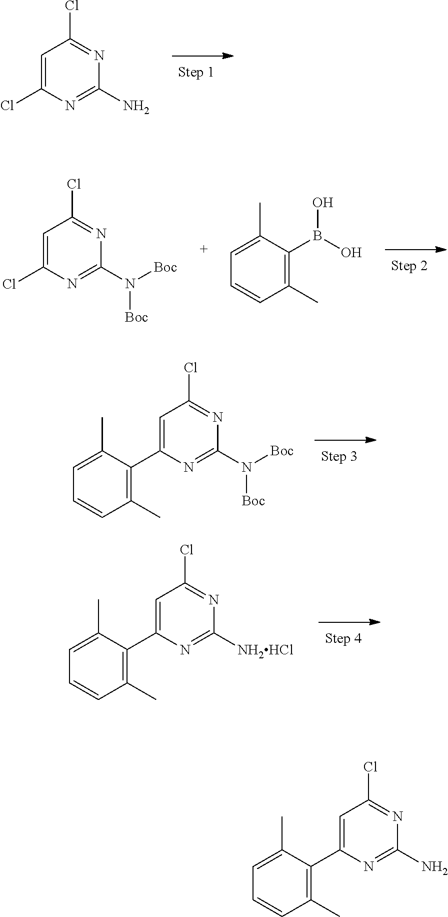

### Step 1: tert-Butyl N-tert-butoxycarbonyl-N-(4,6-dichloropyrimidin-2-yl)carbamate

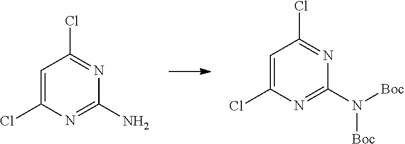 [SMILES: Nc1nc(Cl)cc(Cl)n1 ; CC(C)(C)OC(=O)N(C(=O)OC(C)(C)C)c1nc(Cl)cc(Cl)n1]

To a solution of 4,6-dichloropyrimidin-2-amine (300 g, 1.829 mol) in DCM (2.1 L) was added (Boc)₂₀ (838 g, 3.840 mol) followed by DMAP (5.6 g, 45.84 mmol). The mixture was stirred at ambient temperature for 6 hours. Additional DMAP (5.6 g, 45.84 mmol) was added and the reaction was continued to stir at ambient temperature for 24 hours. The mixture was diluted with water (2.1 L) and the organic phase separated. The organic phase was washed with water (2.1 L), 2.1 L of brine, dried over MgSO₄, filtered over Celite and concentrated in vacuo affording a light orange oil which had a silt in the slurry. The mixture was diluted with ˜500 mL of heptane and filtered using an M filter. The precipitate (SM) was washed with 250 mL of heptane. The filtrate was concentrated in vacuo affording a thick orange oil which was seeded with solid from a previous experiment and crystallized on standing, affording a light orange hard solid. Tert-butyl N-tert-butoxycarbonyl-N-(4,6-dichloropyrimidin-2-yl)carbamate (645 g, 97%). ¹H NMR (400 MHz, DMSO-d₆) δ 8.07 (s, 1H), 1.44 (s, 18H). ESI-MS m/z calc. 363.07526, found 364.1 (M+1)+; Retention time: 2.12 minutes (LC method A).

### Step 2: tert-Butyl N-tert-butoxycarbonyl-N-[4-chloro-6-(2,6-dimethylphenyl) pyrimidin-2-yl]carbamate

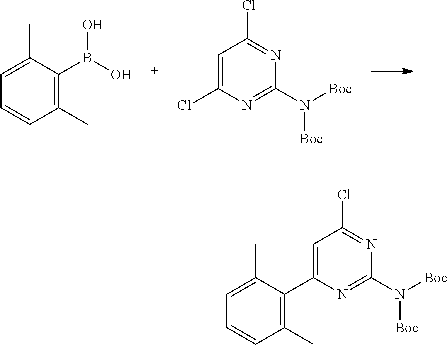 [SMILES: Cc1cccc(C)c1B(O)O ; CC(C)(C)OC(=O)N(C(=O)OC(C)(C)C)c1nc(Cl)cc(Cl)n1 ; Cc1cccc(C)c1-c1cc(Cl)nc(N(C(=O)OC(C)(C)C)C(=O)OC(C)(C)C)n1]

All solvents were degassed prior to use. To a slurry of tert-butyl N-tert-butoxycarbonyl-N-(4,6-dichloropyrimidin-2-yl)carbamate (88 g, 241.6 mmol), (2,6-dimethylphenyl)boronic acid (approximately 36.24 g, 241.6 mmol) and Cs₂CO₃ (approximately 196.8 g, 604.0 mmol) in DME (704 mL) and water (176 mL) were added. Pd(dppf)C₁₂ (approximately 8.839 g, 12.08 mmol) was added and the mixture was vigorously stirred under N2 at 80° C. (reflux) for 1 hour (no starting material remained). The reaction was cooled to ambient temperature and diluted with water (704 mL). The aqueous phase was separated and extracted with EtOAc (704 mL). The organic phase was washed with 700 mL of brine, dried over MgSO₄, filtered and concentrated in vacuo. The crude product was chromatographed on a 1500 g silica gel column eluting with 0-30% EtOAc/hexanes. The product fractions (eluted at 15% EtOAc) were combined and concentrated in vacuo affording the product as a clear oil which crystallized on standing. Tert-butyl N-tert-butoxycarbonyl-N-[4-chloro-6-(2,6-dimethylphenyl)pyrimidin-2-yl]carbamate (81.3 g, 78%). ¹H NMR (400 MHz, DMSO-d₆) δ 7.88 (s, 1H), 7.30 (dd, J=8.2, 7.0 Hz, 1H), 7.21-7.16 (m, 2H), 2.03 (s, 6H), 1.38 (s, 18H). ESI-MS m/z calc. 433.17682, found 434.1 (M+1)+; Retention time: 2.32 minutes (LC method A).

### Step 3: 4-Chloro-6-(2,6-dimethylphenyl)pyrimidin-2-amine (hydrochloride salt)

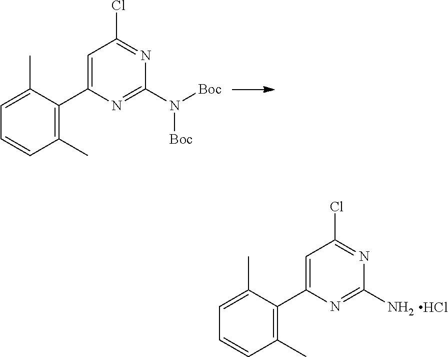 [SMILES: Cc1cccc(C)c1-c1cc(Cl)nc(N(C(=O)OC(C)(C)C)C(=O)OC(C)(C)C)n1 ; Cc1cccc(C)c1-c1cc(Cl)nc(N)n1 ; Cl]

tert-Butyl N-tert-butoxycarbonyl-N-[4-chloro-6-(2,6-dimethylphenyl) pyrimidin-2-yl]carbamate (514.8 g, 915.9 mmol) was dissolved in dichloromethane (4 L). Hydrogen chloride in p-dioxane (1 L, 4 mol) was added and the mixture was stirred overnight at room temperature. The resulting precipitate was collected by vacuum filtration and dried in vacuo to obtain 4-chloro-6-(2,6-dimethylphenyl)pyrimidin-2-amine hydrochloride (213.5 g, 64%) as a white solid (213.5 g, 82%). ¹H NMR (250 MHz, DMSO-d₆) δ 7.45-6.91 (m, 3H), 6.73 (s, 1H), 2.08 (s, 6H). ESI-MS m/z calc. 233.072, found 234.1 (M+1)+; Retention time: 2.1 minutes (LC Method C).

### Step 4: 4-Chloro-6-(2,6-dimethylphenyl)pyrimidin-2-amine

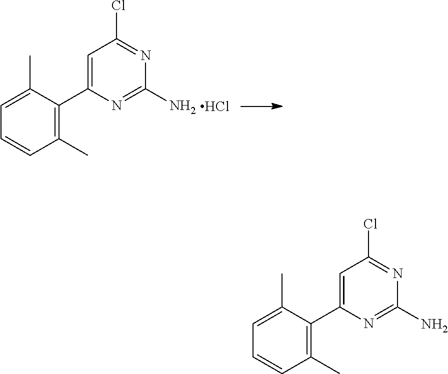 [SMILES: Cc1cccc(C)c1-c1cc(Cl)nc(N)n1 ; Cl ; Cc1cccc(C)c1-c1cc(Cl)nc(N)n1]

4-Chloro-6-(2,6-dimethylphenyl)pyrimidin-2-amine (hydrochloride salt) (166 g, 614.5 mmol) and 4-chloro-6-(2,6-dimethylphenyl)pyrimidin-2-amine (hydrochloride salt) (30 g, 111.0 mmol) were suspended in DCM (2.5 L), treated with NaOH (725 mL of 1 M, 725.0 mmol) and stirred at room temperature for 1 hour. The mixture was transferred into a separatory funnel and left standing over night. The DCM phase was separated and the aqueous phase with insoluble material was extracted twice more with DCM (2×500 mL). The combined brown DCM phases were stirred with magnesium sulfate and charcoal for 1 hour, filtered and the yellow solution concentrated to a volume of ˜ 500 mL. The solution was diluted with heptane (750 mL) and DCM was removed under reduced pressure at 60° C. to give a cream suspension. It was stirred at room temperature for 1 hour, filtered, washed with cold heptane and dried to give 4-chloro-6-(2,6-dimethylphenyl)pyrimidin-2-amine (157 g, 91%) as a cream solid. ¹H NMR (400 MHz, DMSO-d₆) δ 7.28-7.14 (m, 3H), 7.10 (d, J=7.5 Hz, 2H), 6.63 (s, 1H), 2.06 (s, 6H). ESI-MS m/z calc. 233.07198, found 234.0 (M+1)+; Retention time: 1.45 minutes (LC method A).

### B. Preparation of 3-[[4-Chloro-6-(2,6-dimethylphenyl)pyrimidin-2-yl]sulfamoyl]benzoic acid

### Step 1: 3-[[4-Chloro-6-(2,6-dimethylphenyl)pyrimidin-2-yl]sulfamoyl]benzoic acid

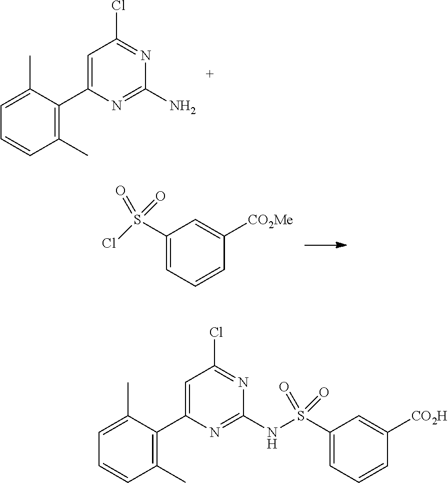 [SMILES: Cc1cccc(C)c1-c1cc(Cl)nc(N)n1 ; "+" ; COC(=O)c1cccc(S(=O)(=O)Cl)c1 ; Cc1cccc(C)c1-c1cc(Cl)nc(NS(=O)(=O)c2cccc(C(=O)O)c2)n1]

4-Chloro-6-(2,6-dimethylphenyl)pyrimidin-2-amine (235 g, 985.5 mmol) was dissolved in MeTHF (2.3 L) and cooled in an ice bath under stirring and nitrogen. To the cold solution methyl 3-chlorosulfonylbenzoate (347 g, 1.479 mol) was added in one portion (seems slightly endothermic) and to the cold pale yellow solution a solution of 2-methyl-butan-2-ol (lithium salt) (875 mL of 3.1 M, 2.712 mol) (in heptane) was added dropwise over 1.25 hour (exothermic, internal temperature from 0 to 10° C.). The ice bath was removed and the greenish solution was stirred for 4 hours at room temperature. To the greenish solution cold HCl (2 L of 1.5 M, 3.000 mol) was added, the phases separated and the organic phase was washed once with water (1 L) and once with brine (500 mL). The aqueous phases were back extracted once with MeTHF (350 mL) and the organic phases were combined. This yellow MeTHF solution of methyl 3-[[4-chloro-6-(2,6-dimethylphenyl)pyrimidin-2-yl]sulfamoyl]benzoate (ESI-MS m/z calc. 431.07065, found 432.0 (M+1)+; Retention time: 1.81 minutes) was treated with NaOH (2.3 L of 2 M, 4.600 mol) and stirred at room temperature for 1 hour. The phases were separated and the NaOH phase was washed twice with MeTHF (2×500 mL) and the combined organic phases were extracted once with 2 M NaOH (1×250 mL). The combined NaOH phases were combined, stirred in an ice bath and slowly acidified by addition of HCl (416 mL of 36% w/w, 4.929 mol) while keeping the internal temperature between 10 and 20° C. At the end of the addition (pH ˜5-6) the final pH was adjusted to 2-3 by addition of solid citric acid. The formed yellow tacky suspension was stirred at room temperature over night to give a cream crisp suspension. The solid was collected by filtration, washed with plenty of water and sucked dry for 3 hours. The solid was dried under reduced pressure with a nitrogen leak at 45-50° C. for 12 hours 3-[[4-chloro-6-(2,6-dimethylphenyl)pyrimidin-2-yl]sulfamoyl]benzoic acid (395 g, 96%) was isolated as an off-white solid. ¹H NMR (400 MHz, DMSO-d₆) δ 13.44 (s, 1H), 12.46 (s, 1H), 8.48-8.39 (m, 1H), 8.25-8.15 (m, 1H), 8.15-8.08 (m, 1H), 7.68 (t, J=7.8 Hz, 1H), 7.31(s, 1H), 7.28-7.18 (m, 1H), 7.10 (d, J=7.6 Hz, 2H), 1.84 (s, 6H). ESI-MS m/z calc. 417.055, found 418.0 (M+1)+; Retention time: 1.56 minutes. (LC method A).

### V. Synthesis of Compounds

### Example 1: Preparation of N-[4-[[6-(Cyclopentylamino)-3-pyridyl]oxy]-6-(2,6-dimethylphenyl)pyrimidin-2-yl]-1-methyl-pyrazole-4-sulfonamide (Compound 1)

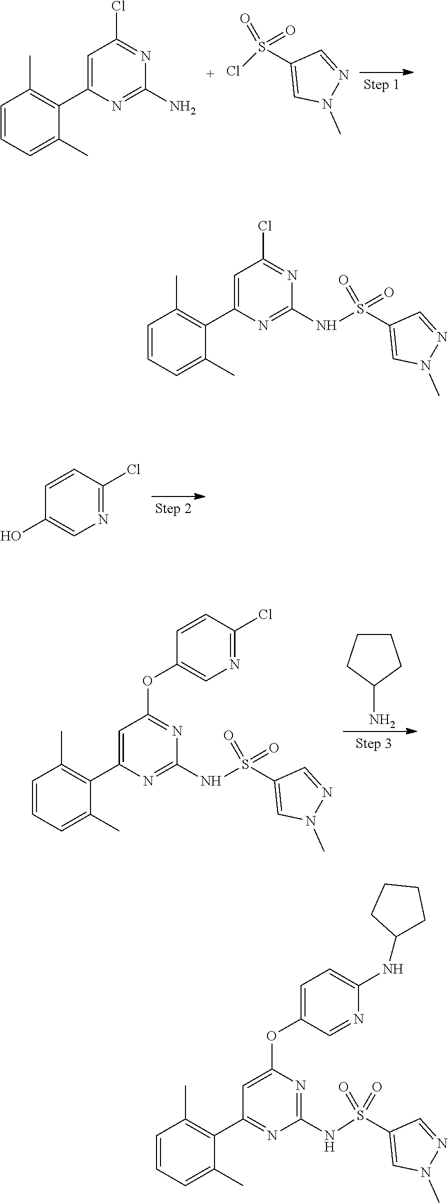

### Step 1: N-[4-Chloro-6-(2,6-dimethylphenyl)pyrimidin-2-yl]-1-methyl-1H-pyrazole-4-sulfonamide

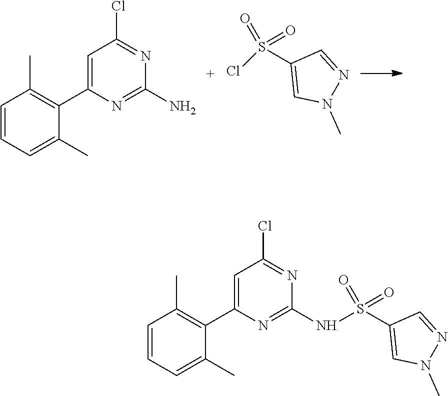

To a suspension of sodium hydride (60% in mineral oil, 6.34 g, 0.159 mol) in anhydrous tetrahydrofuran (50 mL) was added a solution of 4-chloro-6-(2,6-dimethylphenyl)pyrimidin-2-amine (10.6 g, 0.0453 mol) in anhydrous tetrahydrofuran (50 mL) dropwise at 0° C. The reaction was allowed to stir at room temperature for half an hour. A solution of 1-methyl-1H-pyrazole-4-sulfonyl chloride (13.1 g, 0.0725 mol) in anhydrous tetrahydrofuran (30 mL) was added to the reaction mixture dropwise at 0° C. and stirred at this temperature for 1 hour. The reaction was quenched with ice water (150 mL). Dichloromethane (250 mL) was added to the solution. The two layers were separated, and the aqueous layer was extracted with dichloromethane (3×150 mL). The combined organic layers were washed with brine (100 mL), dried over anhydrous sodium sulfate, and concentrated under vacuum. The reside was purified by silica gel column chromatography eluting with methanol in dichloromethane (0% to 10%) to give N-[4-chloro-6-(2,6-dimethylphenyl)pyrimidin-2-yl]-1 -methyl-1H-pyrazole-4-sulfonamide as a white solid (10.3 g, 60%).

The desired product (6.0 g) was dissolved in ethyl acetate (100 mL). Metal scavenger SiliaMetS® Thiol (6.0 g) was added to the solution. The solution was shaken at room temperature for 1 hour. The metal scavenger was filtered off and washed with ethyl acetate. The filtrate was concentrated under vacuum. The residue was triturated with hexane (120 mL). The solid was collected by filtration and dried under vacuum to afford N-[4-chloro-6-(2,6-dimethylphenyl)pyrimidin-2-yl]-1-methyl-1H-pyrazole-4-sulfonamide as a white solid (5.1 g, 85% recovery). ¹H NMR (250 MHz, (CD₃)₂SO) δ 12.1 (s, br, 1H); 8.28 (s, 1H); 7.76 (s, 1H); 7.31 (s, 1H); 7.26 (m, 1H); 7.16 (d, J=7.5 Hz, 2H); 3.82 (s, 3H), 1.96 (s, 6H). ESI-MS m/z calc. 377.1, 379.1, found 377.9, 380.0 (M+H)*. Ret time: 4.06 minutes. (LC method B).

### Step 2: N-[4-[(6-Chloro-3-pyridyl)oxy]-6-(2,6-dimethylphenyl)pyrimidin-2-yl]-1 -methyl-pyrazole-4-sulfonamide

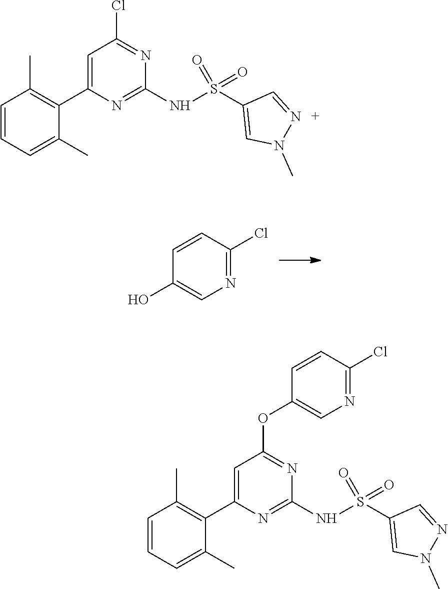

To a solution of N-[4-chloro-6-(2,6-dimethylphenyl)pyrimidin-2-yl]-1-methyl-1H-pyrazole-4-sulfonamide (2.82 g, 7.46 mmol) and 6-chloropyridin-3-ol (1.34 g, 10.3 mmol) in acetonitrile (30 mL) was added potassium carbonate (2.21 g, 15.88 mmol) at room temperature. The reaction mixture was stirred at reflux for 2 days. After cooling down to room temperature, the solid was filtered off, and washed with acetonitrile. The combined filtrate was concentrated under vacuum. The residue was purified by silica gel chromatography eluting with methanol in dichloromethane (0% to 10%). The collected fractions were combined and concentrated under vacuum. The residue was triturated with 50% ethyl acetate in hexanes. The solid was collected by filtration and dried under vacuum to afford N-[4-[(6-chloro-3-pyridyl)oxy]-6-(2,6-dimethylphenyl)pyrimidin-2-yl]-1-methyl-pyrazole-4-sulfonamide as a white solid (2.16 g, 61%). ¹H NMR (250 MHz, (CD₃)₂SO) δ 11.8 (s, br, 1H); 8.49 (d, J=2.8 Hz, 1H); 7.93 (dd, J=2.8 Hz, 8.5 Hz 1H); 7.81 (s, 1H); 7.71 (d, J=8.8 Hz, 1H); 7.26 (m, 2H); 7.16 (d, J=7.3 Hz, 2 H), 6.79 (s, 1H), 3.78 (s, 3H), 2.04 (s, 6H). ESI-MS m/z calc. 470.1, 472.1, found 471.1, 473.0 (M+H)⁺. Ret time: 4.22 min (LC method B).

### Step 3: N-[4-[[6-(Cyclopentylamino)-3-pyridyl]oxy]-6-(2,6-dimethylphenyl)pyrimidin-2-yl]-1-methyl-pyrazole-4-sulfonamide

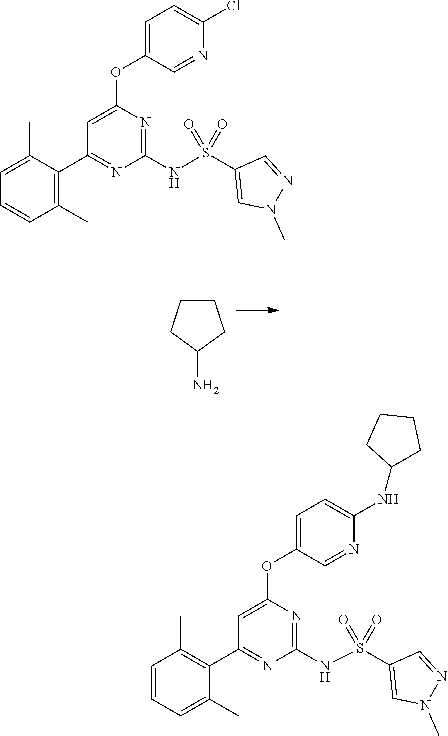 [SMILES: Cc1cccc(C)c1-c1cc(Oc2ccc(Cl)nc2)nc(NS(=O)(=O)c2cnn(C)c2)n1 ; NC1CCCC1 ; Cc1cccc(C)c1-c1cc(Oc2ccc(NC3CCCC3)nc2)nc(NS(=O)(=O)c2cnn(C)c2)n1]

To a dioxane (0.8 mL) solution of Pd₂(dba)₃ (7.1 mg, 0.007753 mmol) and RuPhos (7 mg, 0.01500 mmol) was added N-[4-[(6-chloro-3-pyridyl)oxy]-6-(2,6-dimethylphenyl)pyrimidin-2-yl]-1-methyl-pyrazole-4-sulfonamide (15 mg, 0.03185 mmol), cyclopentanamine (approximately 4.068 mg, 4.714 μL, 0.04778 mmol), and sodium tert-butoxide (approximately 9.183 mg, 0.09555 mmol) sequentially and the mixture was sparged with nitrogen and sonicated for 5 minutes. The reaction mixture was stirred at 50° C. for 30 minutes. The solution was filtered, concentrated in vacuo and the resulting residues dissolved in 1.5 mL DMSO, and purified by reverse phase chromatography using a 15 minutes gradient of 20% MeCN in water to 80% MeCN to give N-[4-[[6-(cyclopentylamino)-3-pyridyl]oxy]-6-(2,6-dimethylphenyl)pyrimidin-2-yl]-1-methyl-pyrazole-4-sulfonamide (hydrochloride salt) (5.2 mg, 29%)ESI-MS m/z calc. 519.20526, found 520.49 (M+1)+; Retention time: 1.16 minutes (LC method A).

### Example 2: Preparation of N-[4-(2,6-dimethylphenyl)-6-[(6-isopentyloxy-3-pyridyl)oxy]pyrimidin-2-yl]-1-methyl-pyrazole-4-sulfonamide (Compound 2)

### Step 1: N-[4-(2,6-Dimethylphenyl)-6-[(6-isopentyloxy-3-pyridyl)oxy]pyrimidin-2-yl]-1-methyl-pyrazole-4-sulfonamide

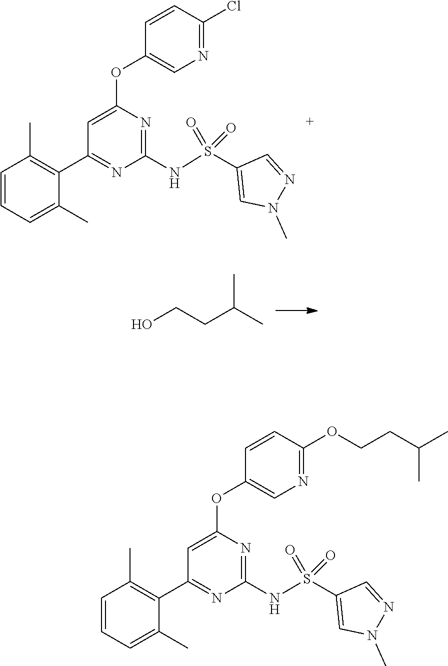

A solution of N-[4-[(6-chloro-3-pyridyl)oxy]-6-(2,6-dimethylphenyl)pyrimidin-2-yl]-1-methyl-pyrazole-4-sulfonamide (25 mg, 0.05309 mmol), 3-methylbutan-1-ol (18 mg, 0.2042 mmol), [2-(2-aminoethyl)phenyl]-chloro-palladium;ditert-butyl-[2-(2,4,6-triisopropylphenyl)phenyl]phosphane (XPhos Pd G1)(approximately 3.646 mg, 0.005309mmol), and Cs₂CO₃ (approximately 69.20 mg, 0.2124 mmol) in dioxane (0.8 mL) was sparged with nitrogen and stirred at room temperature for 30 minutes. The solution was filtered, concentrated in vacuo and the resulting residue was dissolved in 1.5 mL MeOH, and purified by reverse phase chromatography using a 15 minute gradient of 20% MeCN in water to 80% MeCN in water to give N-[4-(2,6-dimethylphenyl)-6-[(6-isopentyloxy-3-pyridyl)oxy]pyrimidin-2-yl]-1-methyl-pyrazole-4-sulfonamide (4.9 mg, 18%). ESI-MS m/z calc. 522.2049, found 523.47 (M+1)⁺; Retention time: 1.99 minutes (LC method A).

### Example 3: Preparation of N-[4-(2,6-dimethylphenyl)-6-[[5-fluoro-6-(4-methylpiperazin-1-yl)-3-pyridyl]oxy]pyrimidin-2-yl]-1-methyl-pyrazole-4-sulfonamide (Compound 3)

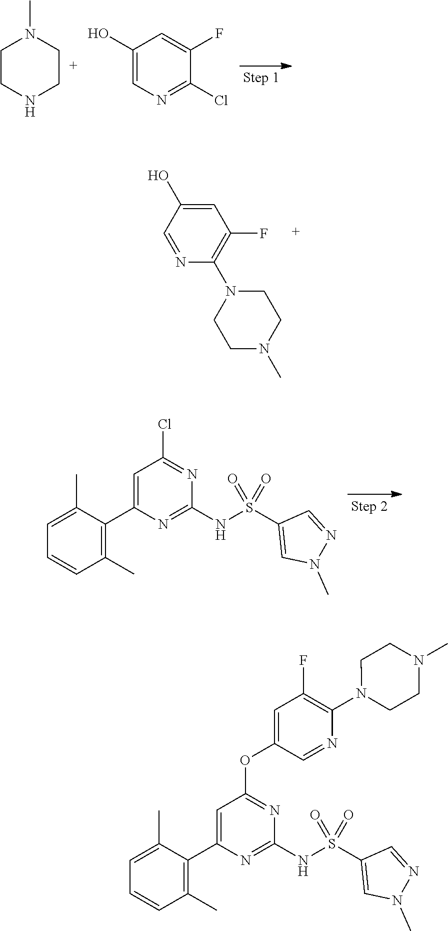

### Step 1: 5-Fluoro-6-(4-methylpiperazin-1-yl)pyridin-3-ol

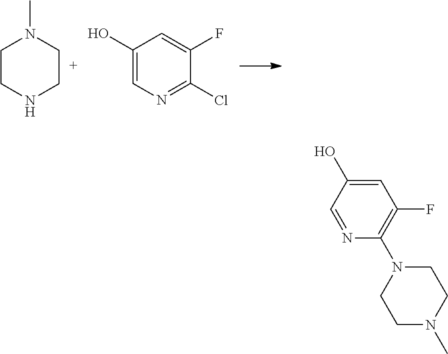

A dioxane (15 mL) mixture of 6-chloro-5-fluoro-pyridin-3-ol (408.5 mg, 2.769 mmol), 1-methylpiperazine (831.5 mg, 8.302 mmol), chloro(2-di-t-butylphosphino-2′, 4′, 6′-tri-i-propyl-1,1′-biphenyl)[2-(2-aminoethyl)phenyl] palladium(II) [t-BuXPhos Palladacycle Gen. 1](381.2 mg, 0.5551 mmol), and sodium tert-butoxide (1.3275 g, 13.81 mmol) was sparged with nitrogen for 10 minutes and then heated to 50° C. for 2 hours. The reaction mixture was then cooled to room temperature and HCl (13.5 mL of 1 M, 13.50 mmol) was added to bring the pH to ˜8. The reaction mixture was diluted with ethyl acetate (20 mL) and the organic layer was separated. The aqueous layer was extracted with ethyl acetate (2×15 mL) and the combined organic layer was washed with water (10 mL) and then dried over anhydrous sodium sulfate, filtered, and concentrated in vacuo. The residue was purified by flash column chromatography on silica gel using a 24 g column and eluting with 0-10% MeOH in DCM over 20 minutes to give 5-fluoro-6-(4-methylpiperazin-1-yl)pyridin-3-ol (194 mg, 33%). ESI-MS m/z calc. 211.11209, found 212.12 (M+1)+; Retention time: 0.49 minutes (LC method A).

### Step 2: N-[4-(2,6-Dimethylphenyl)-6-[[5-fluoro-6-(4-methylpiperazin-1-yl)-3-pyridyl]oxy]pyrimidin-2-yl]-1-methyl-pyrazole-4-sulfonamide

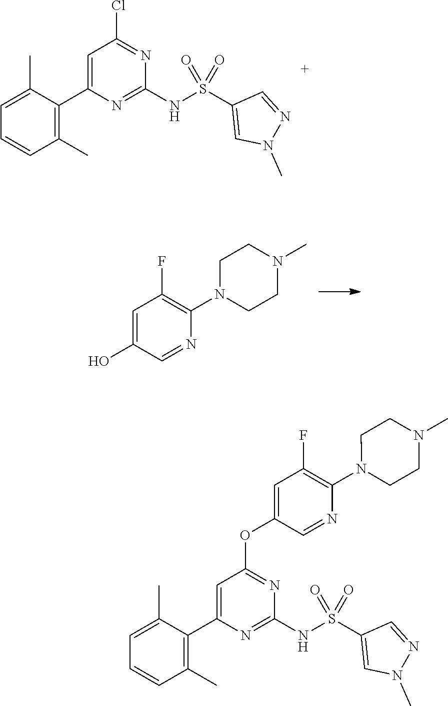 [SMILES: Cc1cccc(C)c1-c1cc(Cl)nc(NS(=O)(=O)c2cnn(C)c2)n1 ; CN1CCN(c2ncc(O)cc2F)CC1 ; Cc1cccc(C)c1-c1cc(Oc2cnc(N3CCN(C)CC3)c(F)c2)nc(NS(=O)(=O)c2cnn(C)c2)n1]

A NMP (0.5 mL) mixture of 5-fluoro-6-(4-methylpiperazin-1-yl)pyridin-3-ol (25.1 mg, 0.1188 mmol), N-[4-chloro-6-(2,6-dimethylphenyl)pyrimidin-2-yl]-1-methyl-pyrazole-4-sulfonamide (14.8 mg, 0.03917 mmol), and Cs₂CO₃ (51.3 mg, 0.1574 mmol) was heated to 100° C. for 16 hours and then cooled to room temperature. The solution was filtered and the filtrate was dissolved in 0.8 mL MeOH, and purified by reverse phase chromatography using a 15 minutes gradient of 20% MeCN in water to 80% MeCN (HCl modifier) to give N-[4-(2,6-dimethylphenyl)-6-[[5-fluoro-6-(4-methylpiperazin-1-yl)-3-pyridyl]oxy]pyrimidin-2-yl]-1 -methyl-pyrazole-4-sulfonamide (hydrochloride salt) (7.5 mg, 33%). ESI-MS m/z calc. 552.2067, found 553.4 (M+1)+; Retention time: 1.14 minutes (LC method A).

### Example 4: Preparation of Compound 4

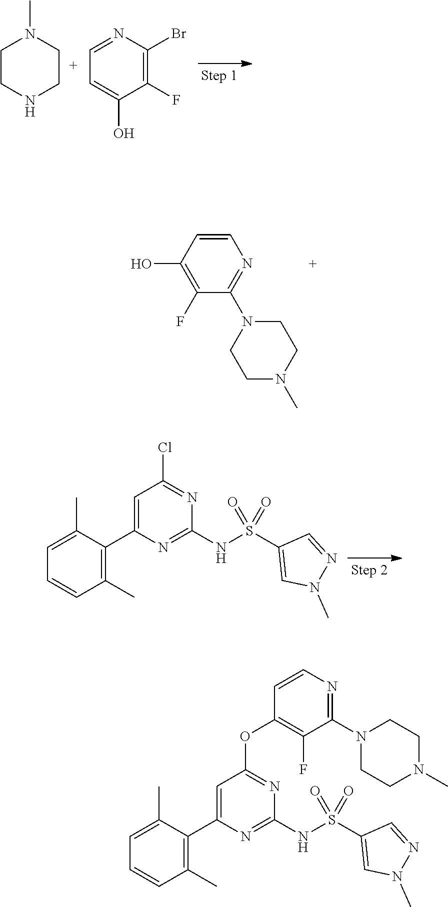

### Step 1: 3-Fluoro-2-(4-methylpiperazin-1-yl)pyridin-4-ol

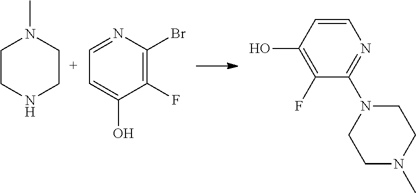

A dioxane (13.54 mL) mixture of 1-methylpiperazine (815.1 mg, 8.138 mmol), 2-bromo-3-fluoro-pyridin-4-ol (519.9 mg, 2.708 mmol), chloro(2-di-t-butylphosphino-2′, 4′, 6′-tri-i-propyl-1,1′-biphenyl)[2-(2-aminoethyl)phenyl] palladium(II) [t-BuXPhos Palladacycle Gen. 1](384.6 mg, 0.5601 mmol) and sodium tert-butoxide (1.285 g, 13.37 mmol) was stirred for 30 minutes at room temperature. HCl (12 mL of 1 M, 12.00 mmol) was added, followed by ethyl acetate (10 mL). The pH of aqueous layer was ˜7-8. The organic layer was separated, and the product was extracted from the aqueous layer (2×5 mL) with ethyl acetate. The combined organic layers were washed with water (5 mL) and then brine (5 mL) and dried with anhydrous sodium sulfate, filtered, and concentrated in vacuo. The crude residue was dissolved in DMSO (3 mL) and a 400 μL sample was purified by reverse phase chromatography using a 15 minute gradient of 1% MeCN in water to 30% MeCN (HCl modifier). This purification was repeated four times and the combined fractions afforded 3-fluoro-2-(4-methylpiperazin-1-yl)pyridin-4-ol (hydrochloride salt) (40.0 mg, 6%). ESI-MS m/z calc. 211.11209, found 212.1 (M+1)⁺; Retention time: 0.17 minutes.

### Step 2: N-[4-(2,6-Dimethylphenyl)-6-[[3-fluoro-2-(4-methylpiperazin-1-yl)-4-pyridyl]oxy]pyrimidin-2-yl]-1-methyl-pyrazole-4-sulfonamide

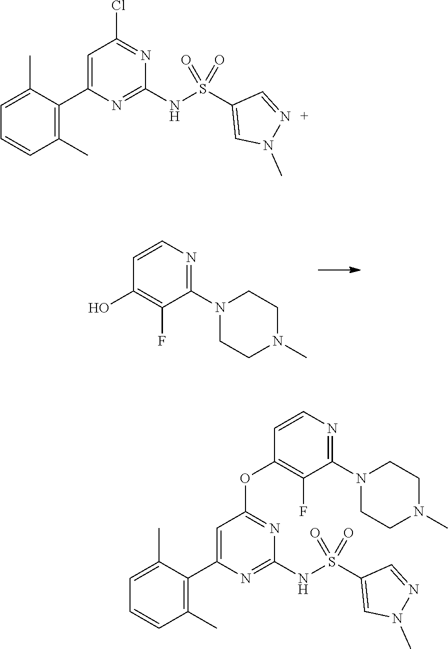

A NMP (0.5 mL) mixture of N-[4-chloro-6-(2,6-dimethylphenyl)pyrimidin-2-yl]-1 -methyl-pyrazole-4-sulfonamide (13.1 mg, 0.03467 mmol), Cs₂CO₃ (76 mg, 0.2333 mmol), and 3-fluoro-2-(4-methylpiperazin-1-yl)pyridin-4-ol (hydrochloride salt) (25.3 mg, 0.1021 mmol) was stirred at 110° C. for 16 hours and then cooled to room temperature. The solution was filtered and the filtrate was dissolved in 0.8 mL MeOH, and purified by reverse phase chromatography using a 15 minute gradient of 20% MeCN in water to 80% MeCN (HCl modifier) to give N-[4-(2,6-dimethylphenyl)-6-[[3-fluoro-2-(4-methylpiperazin-1-yl)-4-pyridyl]oxy]pyrimidin-2-yl]-1-methyl-pyrazole-4-sulfonamide (hydrochloride salt) (1.8 mg, 9%) ESI-MS m/z calc. 552.2067, found 553.33 (M+1)+; Retention time: 1.27 minutes (LC method A).

### Example 5: Preparation of Compound 5

### Step 1: N-[4-(2,6-Dimethylphenyl)-6-[2-(4-methylpiperazin-1-yl)pyrimidin-5-yl]oxy-pyrimidin-2-yl]-1-methyl-pyrazole-4-sulfonamide

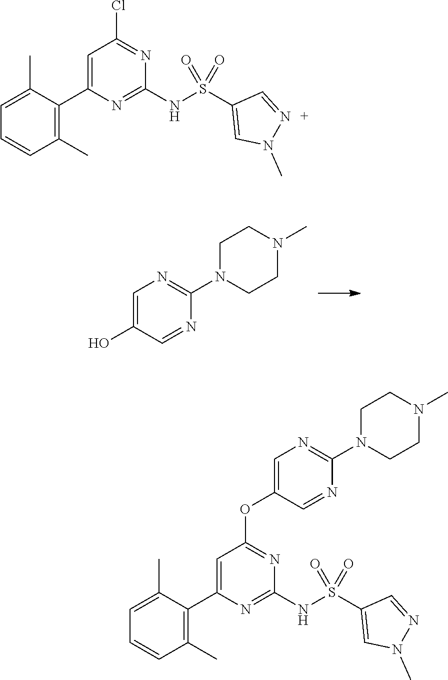

To a 3-mL vial equipped with a magnetic stir bar, N-[4-chloro-6-(2,6-dimethylphenyl)pyrimidin-2-yl]-1-methyl-pyrazole-4-sulfonamide (54.0 mg, 0.1401 mmol), 2-(4-methylpiperazin-1-yl)pyrimidin-5-ol (40.1 mg, 0.2065 mmol), K₂CO₃ (50.0 mg, 0.3618 mmol) and NMP (800 μL) were added. This slurry was stirred at 120° C. for 19 hours. The reaction mixture was then cooled to room temperature, diluted with MeOH (400 μL), filtered and purified by reverse phase HPLC (1-70% acetonitrile in water using HCl as a modifier) to give a combined 58.5 mg of ˜70% pure product and ˜30% side product, N-[4-(2,6-dimethylphenyl)-6-hydroxy-pyrimidin-2-yl]-1-methyl-pyrazole-4-sulfonamide. 7.5 mg out of this mixture was re-purified by preparative TLC (one full silica plate, 20 cm×20 cm, 250 m thickness, 60 Å particle size, 10% methanol/EtOAc, UV active band at baseline) to give N-[4-(2,6-dimethylphenyl)-6-[2-(4-methylpiperazin-1-yl)pyrimidin-5-yl]oxy-pyrimidin-2-yl]-1 -methyl-pyrazole-4-sulfonamide (4.9 mg, 7%) ESI-MS m/z calc. 535.2114, found 536.4 (M+1)+; Retention time: 1.14 minutes (LC method A).

### Example 6: Characterization of Compounds 6-25

The compounds in the following tables were prepared in a manner analogous to that described above using commercially available reagents and intermediates described herein.

| | | LCMS | | | |
|---|---|---|---|---|---|
| | | Room | | | |
| Com- | | Temper- | | | |
| pound | | ature | Calc. | | LCMS |
| number | Structure | (min) | mass | M + 1 | Method |
| 6 | 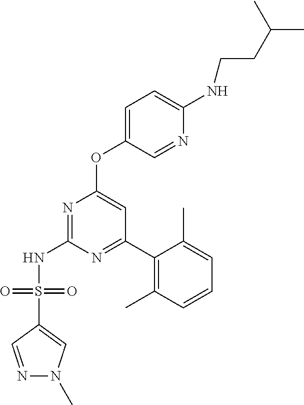 [SMILES: Cc1cccc(C)c1-c1cc(Oc2ccc(NCCC(C)C)nc2)nc(NS(=O)(=O)c2cnn(C)c2)n1] | 1.25 | 521.221 | 522.53 | A |
| 7 | 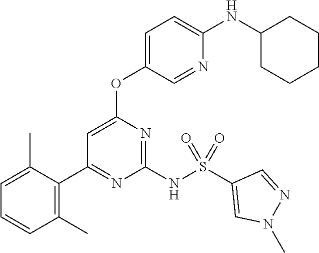 [SMILES: Cc1cccc(C)c1-c1cc(Oc2ccc(NC3CCCCC3)nc2)nc(NS(=O)(=O)c2cnn(C)c2)n1] | 1.24 | 533.221 | 534.5 | A |
| 8 | 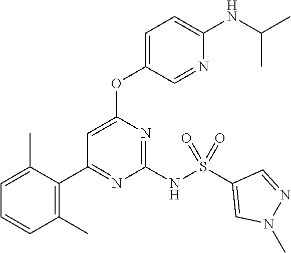 [SMILES: Cc1cccc(C)c1-c1cc(Oc2ccc(NC(C)C)nc2)nc(NS(=O)(=O)c2cnn(C)c2)n1] | 1.06 | 493.19 | 494.48 | A |
| 9 | 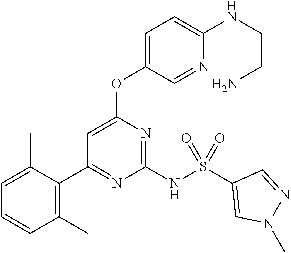 [SMILES: Cc1cccc(C)c1-c1cc(Oc2ccc(NCCN)nc2)nc(NS(=O)(=O)c2cnn(C)c2)n1] | 0.89 | 494.185 | 495.42 | A |
| 10 | 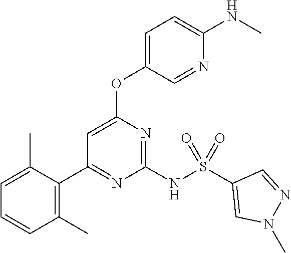 [SMILES: CNc1ccc(Oc2cc(-c3c(C)cccc3C)nc(NS(=O)(=O)c3cnn(C)c3)n2)cn1] | 0.94 | 465.158 | 466.4 | A |
| 11 | 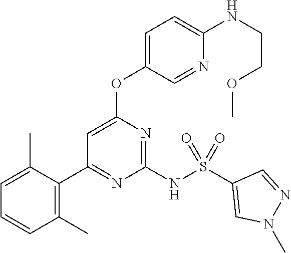 [SMILES: COCCNc1ccc(Oc2cc(-c3c(C)cccc3C)nc(NS(=O)(=O)c3cnn(C)c3)n2)cn1] | 1.02 | 509.185 | 510.48 | A |
| 12 | 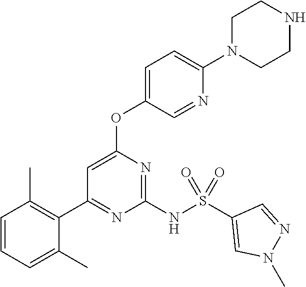 [SMILES: Cc1cccc(C)c1-c1cc(Oc2ccc(N3CCNCC3)nc2)nc(NS(=O)(=O)c2cnn(C)c2)n1] | 1.09 | 520.201 | 521.51 | A |
| 13 | 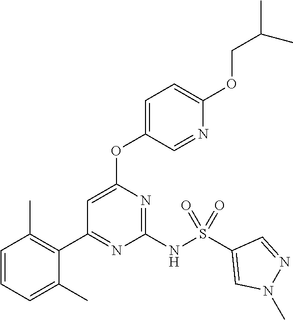 [SMILES: Cc1cccc(C)c1-c1cc(Oc2ccc(OCC(C)C)nc2)nc(NS(=O)(=O)c2cnn(C)c2)n1] | 1.9 | 508.189 | 509.5 | A |
| 14 | 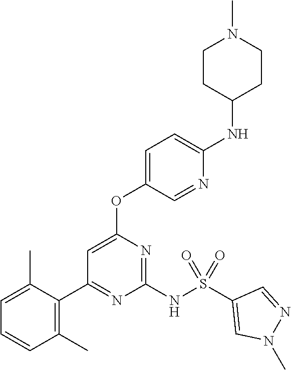 [SMILES: Cc1cccc(C)c1-c1cc(Oc2ccc(NC3CCN(C)CC3)nc2)nc(NS(=O)(=O)c2cnn(C)c2)n1] | 0.89 | 548.232 | 549.52 | A |
| 15 | 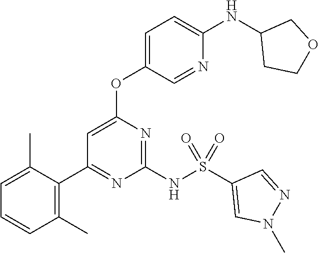 [SMILES: Cc1cccc(C)c1-c1cc(Oc2ccc(NC3CCOC3)nc2)nc(NS(=O)(=O)c2cnn(C)c2)n1] | 1.04 | 521.185 | 522.46 | A |
| 16 | 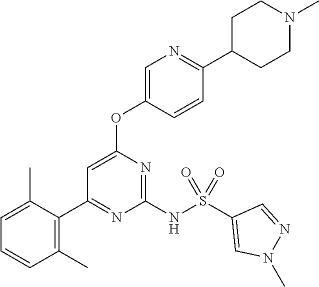 [SMILES: Cc1cccc(C)c1-c1cc(Oc2ccc(C3CCN(C)CC3)nc2)nc(NS(=O)(=O)c2cnn(C)c2)n1] | 1.14 | 533.221 | 534.3 | A |
| 17 | 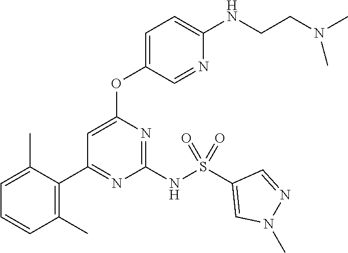 [SMILES: Cc1cccc(C)c1-c1cc(Oc2ccc(NCCN(C)C)nc2)nc(NS(=O)(=O)c2cnn(C)c2)n1] | 0.97 | 522.216 | 523.51 | A |
| 18 | 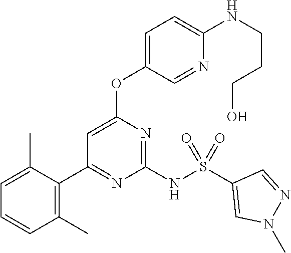 [SMILES: Cc1cccc(C)c1-c1cc(Oc2ccc(NCCCO)nc2)nc(NS(=O)(=O)c2cnn(C)c2)n1] | 0.94 | 509.185 | 510.48 | A |
| 19 | 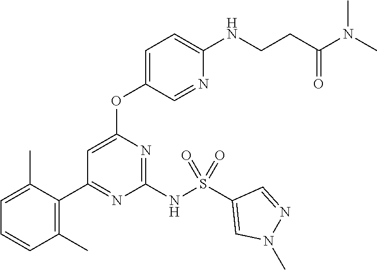 [SMILES: Cc1cccc(C)c1-c1cc(Oc2ccc(NCCC(=O)N(C)C)nc2)nc(NS(=O)(=O)c2cnn(C)c2)n1] | 1.02 | 550.211 | 551.48 | A |
| 20 | 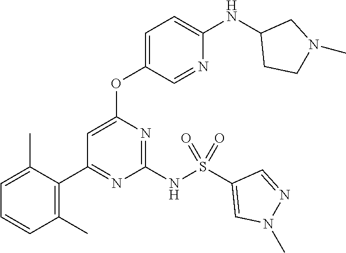 [SMILES: Cc1cccc(C)c1-c1cc(Oc2ccc(NC3CCN(C)C3)nc2)nc(NS(=O)(=O)c2cnn(C)c2)n1] | 0.98 | 534.216 | 535.48 | A |
| 21 | 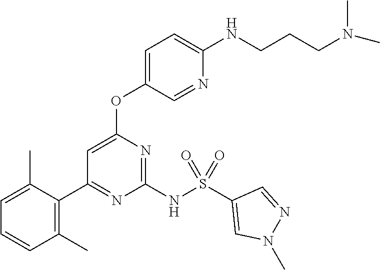 [SMILES: Cc1cccc(C)c1-c1cc(Oc2ccc(NCCCN(C)C)nc2)nc(NS(=O)(=O)c2cnn(C)c2)n1] | 0.85 | 536.232 | 537.58 | A |
| 22 | 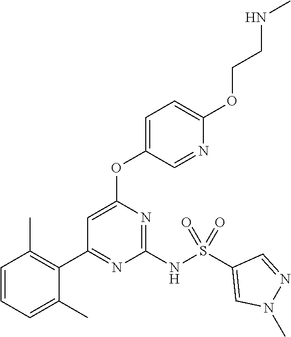 [SMILES: CNCCOc1ccc(Oc2cc(-c3c(C)cccc3C)nc(NS(=O)(=O)c3cnn(C)c3)n2)cn1] | 1.03 | 509.185 | 510.45 | A |
| 23 | 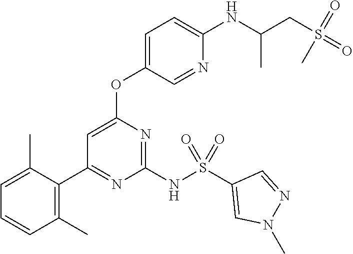 [SMILES: Cc1cccc(C)c1-c1cc(Oc2ccc(NC(C)CS(C)(=O)=O)nc2)nc(NS(=O)(=O)c2cnn(C)c2)n1] | 1.11 | 571.167 | 572.42 | A |
| 24 | 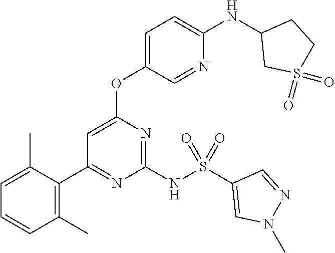 [SMILES: Cc1cccc(C)c1-c1cc(Oc2ccc(NC3CCS(=O)(=O)C3)nc2)nc(NS(=O)(=O)c2cnn(C)c2)n1] | 1.17 | 569.152 | 570.39 | A |
| 25 | 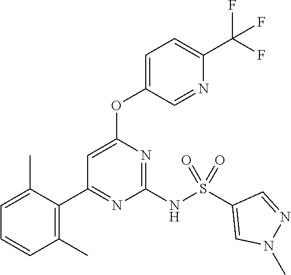 [SMILES: Cc1cccc(C)c1-c1cc(Oc2ccc(C(F)(F)F)nc2)nc(NS(=O)(=O)c2cnn(C)c2)n1] | 1.6 | 504.119 | 505.3 | A |

| Com- | |
|---|---|
| pound | |
| number | NMR |
| 22 | ¹H NMR (400 MHZ, DMSO-d₆) δ 9.01 (s, 2H), 8.21 (d, J = 2.9 Hz, 1H), |
| | 7.82 (dd, J = 8.9, 2.9 Hz, 1H), 7.71 (s, 1H), 7.37-7.19 (m, 2H), 7.14 (d, |
| | J = 7.6 Hz, 2H), 7.01 (d, J = 8.9 Hz, 1H), 6.71 (s, 1H), 4.81-4.30 (m, 2H), |
| | 3.78 (s, 3H), 3.53-3.22 (m, 2H), 2.68-2.53 (m, 3H), 2.04 (s, 6H). |

### Example 7: Preparation of Compound 26

### Step 1: N-[4-(2,6-Dimethylphenyl)-6-[(1R)-1-phenylethoxy]pyrimidin-2-yl]-1 -methyl-pyrazole-4-sulfonamide

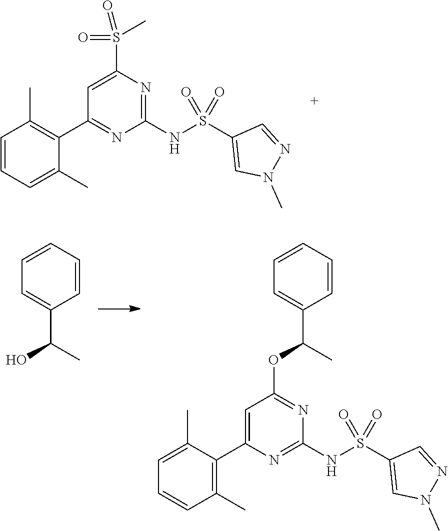

A mixture of N-[4-(2,6-dimethylphenyl)-6-methylsulfonyl-pyrimidin-2-yl]-1-methyl-pyrazole-4-sulfonamide (20 mg, 0.04745 mmol), (1R)-1-phenylethanol (approximately 17.40 mg, 0.1424 mmol) and K₂CO₃ (approximately 26.23 mg, 0.1898 mmol) in NMP (0.4 mL) was stirred at 80° C. for 16 hours. The reaction mixture was diluted with MeOH, filtered and purification by HPLC (1-99% ACN in water (HCl modifier)) gave N-[4-(2,6-dimethylphenyl)-6-[(1R)-1-phenylethoxy]pyrimidin-2-yl]-1-methyl-pyrazole-4-sulfonamide (0.022 g, 13%). ESI-MS m/z calc. 463.16782, found 463.85 (M+1)⁺; Retention time: 1.73 minutes (LC method A).

### Example 8: Preparation of Compound 27

### Step 1: N-[4-(2,2-Dimethylcyclopentoxy)-6-(2,6-dimethylphenyl)pyrimidin-2-yl]-1 -methyl-pyrazole-4-sulfonamide

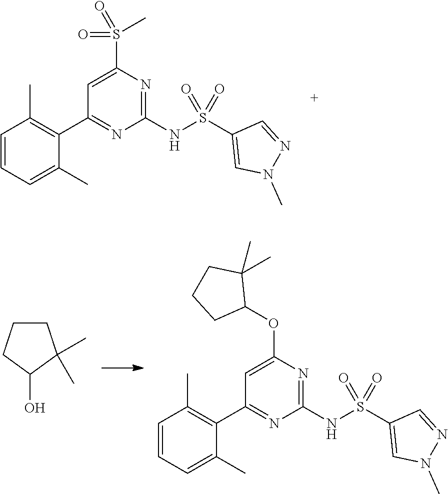

A mixture of N-[4-(2,6-dimethylphenyl)-6-methylsulfonyl-pyrimidin-2-yl]-1-methyl-pyrazole-4-sulfonamide (25 mg, 0.05931 mmol), 2,2-dimethylcyclopentanol (approximately 33.86 mg, 0.2966 mmol) and cesium carbonate (approximately 96.61 mg, 0.2966 mmol) in NMP (0.5 mL) was stirred at 120° C. for 3 hours. The reaction mixture was diluted with DMSO and purified by HPLC (1-99% ACN in water (HCl modifier)) gave N-[4-(2,2-dimethylcyclopentoxy)-6-(2,6-dimethylphenyl)pyrimidin-2-yl]-1-methyl-pyrazole-4-sulfonamide (9.3 mg, 34%) as a white solid. ESI-MS m/z calc. 455.1991, found 456.45 (M+1)⁺; Retention time: 1.87 minutes (LC method A).

### Example 9: Preparation of Compound 28

### Step 1: N-[4-(2,6-Dimethylphenyl)-6-(3-hydroxy-3-methyl-butoxy)pyrimidin-2-yl]-1-methyl-pyrazole-4-sulfonamide

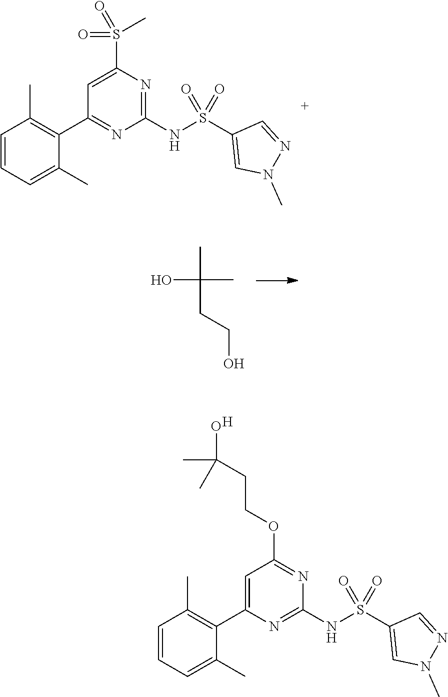

To a 10 mL vial equipped with a magnetic stir bar, N-[4-(2,6-dimethylphenyl)-6-methylsulfonyl-pyrimidin-2-yl]-1-methyl-pyrazole-4-sulfonamide (25.00 mg, 0.05931 mmol) (25.0 mg, 0.0593 mmol), N-methylpyrrolidinone (800 μL) and 3-methylbutane-1,3-diol (23.95 mg, 0.23 mmol) were added, followed by potassium carbonate (31.8 mg, 0.23 mmol). This mixture was stirred at 110° C. for 15 hours. The reaction mixture was then cooled to room temperature, quenched with 1 N HCl (1 mL), and extracted with ethyl acetate (3×1 mL). The combined organic extracts were washed with water (2×2 mL) and saturated aqueous sodium chloride solution (2 mL), then dried over sodium sulfate, filtered, and evaporated in vacuo. Purification by reverse phase HPLC (1-99% acetonitrile in water using HCl as a modifier) gave the desired product, N-[4-(2,6-dimethylphenyl)-6-(3-hydroxy-3-methyl-butoxy)pyrimidin-2-yl]-1-methyl-pyrazole-4-sulfonamide (10.7 mg, 40%). ESI-MS m/z calc. 445.17838, found 446.3 (M+1)⁺; Retention time: 1.29 minutes (LC method A)

### Example 10: Preparation of Compound 29

### Step 1: tert-Butyl 4-[6-(2,6-dimethylphenyl)-2-[(1-methylpyrazol-4-yl)sulfonylamino]pyrimidin-4-yl]oxyazepane-1-carboxylate

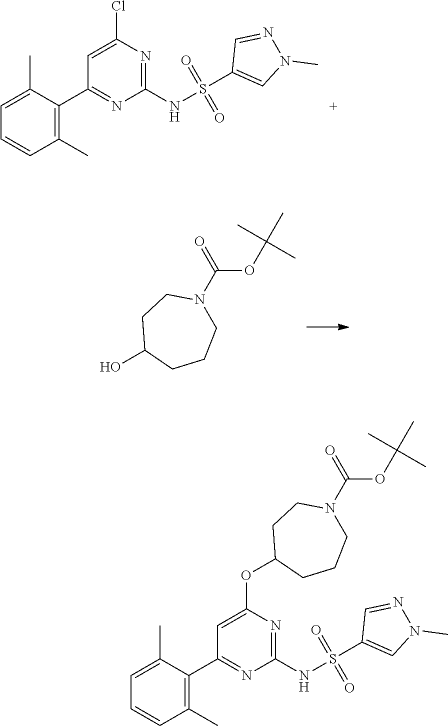 [SMILES: Cc1cccc(C)c1-c1cc(Cl)nc(NS(=O)(=O)c2cnn(C)c2)n1 ; CC(C)(C)OC(=O)N1CCCC(O)CC1 ; Cc1cccc(C)c1-c1cc(OC2CCCN(C(=O)OC(C)(C)C)CC2)nc(NS(=O)(=O)c2cnn(C)c2)n1]

NaH (approximately 48.08 mg of 60% w/w, 1.202 mmol) was added to NMP (5 mL) at 0° C. The mixture was stirred for 45 minutes. then added to N-[4-chloro-6-(2,6-dimethylphenyl)pyrimidin-2-yl]-1-methyl-pyrazole-4-sulfonamide (227 mg, 0.6008 mmol) in NMP (5 mL). The resulting mixture was stirred at 105° C. for 3 hours. The pH of the mixture was adjusted to ˜5 with 1 N HCl, then extracted with ethyl acetate (3×10 mL). The combined organic layers were extracted with brine, dried over Na₂SO₄, concentrated and purified by reverse phase HPLC (HCl, 20-80% ACN-H₂O) to give tert-butyl 4-[6-(2,6-dimethylphenyl)-2-[(1-methylpyrazol-4-yl)sulfonylamino]pyrimidin-4-yl]oxyazepane-1-carboxylate (18.3 mg, 5%). ESI-MS m/z calc. 556.24677, found 557.0 (M+1)⁺; Retention time: 1.79 minutes (LC method A).

### Example 11: Characterization of Compounds 30-56

The compounds in the following tables were prepared in a manner analogous to that described above using commercially available reagents and intermediates described herein.

| | | LCMS Room | | | |
|---|---|---|---|---|---|
| Compound | | Temperature | calc. | | LCMS |
| number | Structure | (min) | mass | M + 1 | Method |
| 30 | 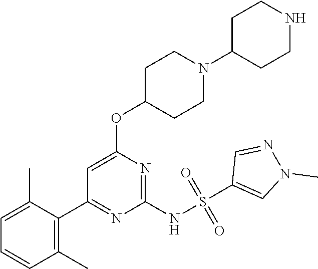 [SMILES: Cc1cccc(C)c1-c1cc(OC2CCN(C3CCNCC3)CC2)nc(NS(=O)(=O)c2cnn(C)c2)n1] | 0.77 | 525.252 | 526.55 | A |
| 31 | 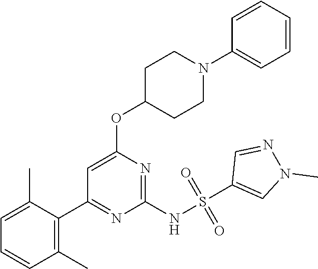 [SMILES: Cc1cccc(C)c1-c1cc(OC2CCN(c3ccccc3)CC2)nc(NS(=O)(=O)c2cnn(C)c2)n1] | 1.32 | 518.21 | 519.51 | A |
| 32 | 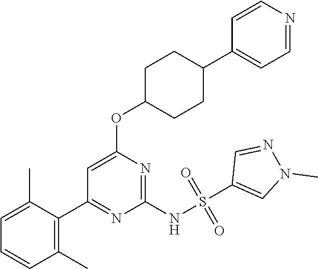 [SMILES: Cc1cccc(C)c1-c1cc(OC2CCC(c3ccncc3)CC2)nc(NS(=O)(=O)c2cnn(C)c2)n1] | 1.18 | 518.21 | 519.51 | A |
| 33 | 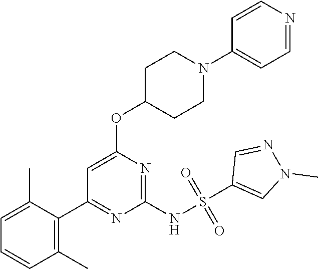 [SMILES: Cc1cccc(C)c1-c1cc(OC2CCN(c3ccncc3)CC2)nc(NS(=O)(=O)c2cnn(C)c2)n1] | 1.09 | 519.205 | 520.49 | A |
| 34 | 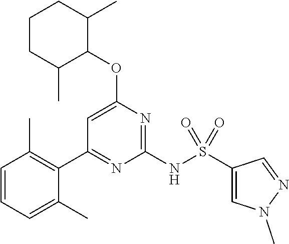 [SMILES: Cc1cccc(C)c1-c1cc(OC2C(C)CCCC2C)nc(NS(=O)(=O)c2cnn(C)c2)n1] | 1.67 | 469.215 | 471.49 | A |
| 35 | 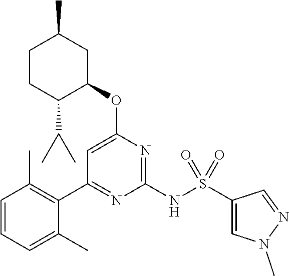 [SMILES: Cc1cccc(C)c1-c1cc(O[C@@H]2C[C@H](C)CC[C@H]2C(C)C)nc(NS(=O)(=O)c2cnn(C)c2)n1] | 2.12 | 497.246 | 498.16 | A |
| 36 | 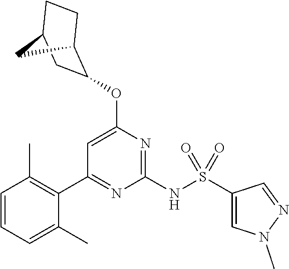 [SMILES: Cc1cccc(C)c1-c1cc(O[C@@H]2C[C@@H]3CC[C@H]2C3)nc(NS(=O)(=O)c2cnn(C)c2)n1] | 1.78 | 453.183 | 454.11 | A |
| 37 | 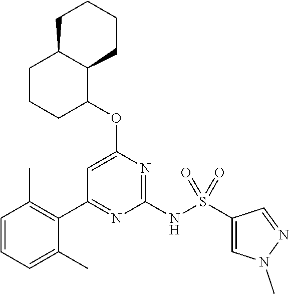 [SMILES: Cc1cccc(C)c1-c1cc(OC2CCC[C@H]3CCCC[C@@H]23)nc(NS(=O)(=O)c2cnn(C)c2)n1] | 2.11 | 495.23 | 497.62 | A |
| 38 | 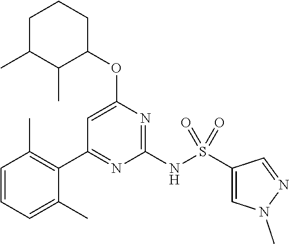 [SMILES: Cc1cccc(C)c1-c1cc(OC2CCCC(C)C2C)nc(NS(=O)(=O)c2cnn(C)c2)n1] | 1.97 | 469.215 | 471.49 | A |
| 39 | 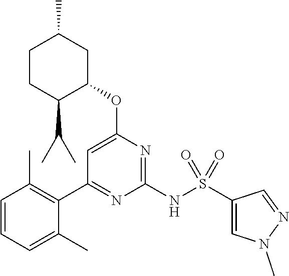 [SMILES: Cc1cccc(C)c1-c1cc(O[C@H]2C[C@@H](C)CC[C@@H]2C(C)C)nc(NS(=O)(=O)c2cnn(C)c2)n1] | 2.12 | 497.246 | 497.91 | A |
| 40 | 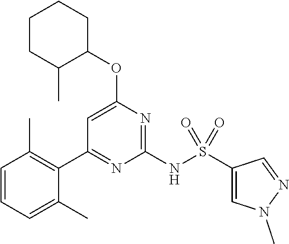 [SMILES: Cc1cccc(C)c1-c1cc(OC2CCCCC2C)nc(NS(=O)(=O)c2cnn(C)c2)n1] | 1.85 | 455.199 | 457.55 | A |
| 41 | 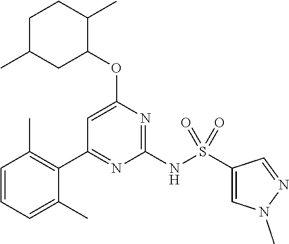 [SMILES: Cc1cccc(C)c1-c1cc(OC2CC(C)CCC2C)nc(NS(=O)(=O)c2cnn(C)c2)n1] | 1.98 | 469.215 | 471.49 | A |
| 42 | 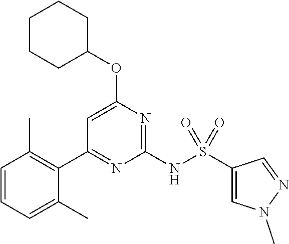 [SMILES: Cc1cccc(C)c1-c1cc(OC2CCCCC2)nc(NS(=O)(=O)c2cnn(C)c2)n1] | 1.75 | 441.183 | 442.41 | A |
| 43 | 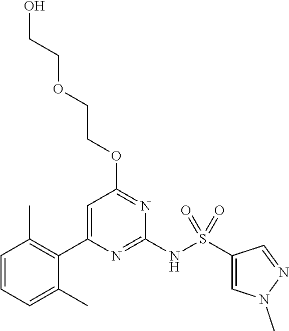 [SMILES: Cc1cccc(C)c1-c1cc(OCCOCCO)nc(NS(=O)(=O)c2cnn(C)c2)n1] | 1.03 | 447.158 | 448.2 | A |
| 44 | 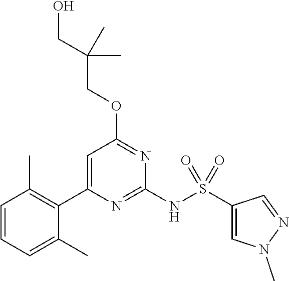 [SMILES: Cc1cccc(C)c1-c1cc(OCC(C)(C)CO)nc(NS(=O)(=O)c2cnn(C)c2)n1] | 1.33 | 445.178 | 446.3 | A |
| 45 | 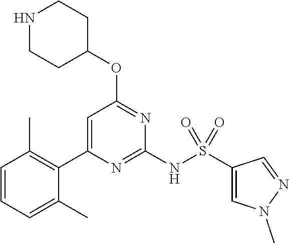 [SMILES: Cc1cccc(C)c1-c1cc(OC2CCNCC2)nc(NS(=O)(=O)c2cnn(C)c2)n1] | 0.815 | 442.179 | 443.1 | A |
| 46 | 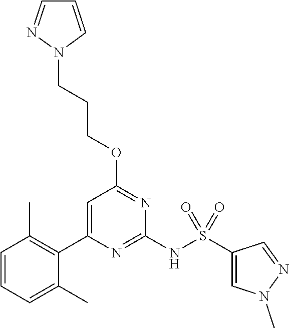 [SMILES: Cc1cccc(C)c1-c1cc(OCCCn2cccn2)nc(NS(=O)(=O)c2cnn(C)c2)n1] | 1.29 | 467.174 | 468.2 | A |
| 47 | 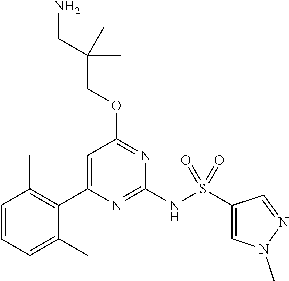 [SMILES: Cc1cccc(C)c1-c1cc(OCC(C)(C)CN)nc(NS(=O)(=O)c2cnn(C)c2)n1] | 0.9 | 444.194 | 445.2 | A |
| 48 | 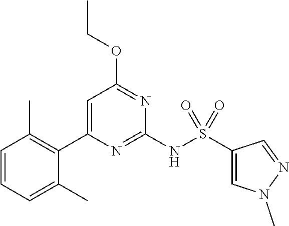 [SMILES: CCOc1cc(-c2c(C)cccc2C)nc(NS(=O)(=O)c2cnn(C)c2)n1] | 1.34 | 387.137 | 388.2 | A |
| 49 | 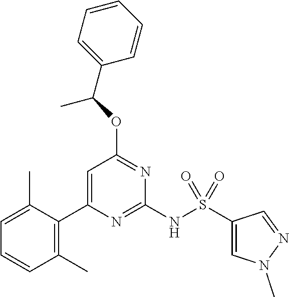 [SMILES: Cc1cccc(C)c1-c1cc(O[C@@H](C)c2ccccc2)nc(NS(=O)(=O)c2cnn(C)c2)n1] | 1.73 | 463.168 | 464.2 | A |
| 50 | 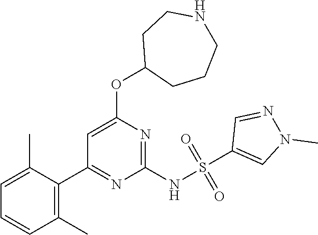 [SMILES: Cc1cccc(C)c1-c1cc(OC2CCCNCC2)nc(NS(=O)(=O)c2cnn(C)c2)n1] | 0.84 | 456.194 | 457 | A |
| 51 | 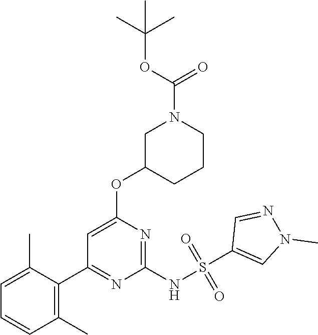 [SMILES: Cc1cccc(C)c1-c1cc(OC2CCCN(C(=O)OC(C)(C)C)C2)nc(NS(=O)(=O)c2cnn(C)c2)n1] | 1.7 | 542.231 | 543 | A |
| 52 | 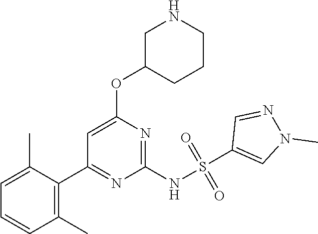 [SMILES: Cc1cccc(C)c1-c1cc(OC2CCCNC2)nc(NS(=O)(=O)c2cnn(C)c2)n1] | 0.81 | 442.179 | 442 | A |
| 53 | 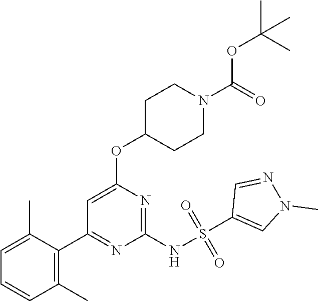 [SMILES: Cc1cccc(C)c1-c1cc(OC2CCN(C(=O)OC(C)(C)C)CC2)nc(NS(=O)(=O)c2cnn(C)c2)n1] | 1.86 | 542.231 | 543 | A |
| 54 | 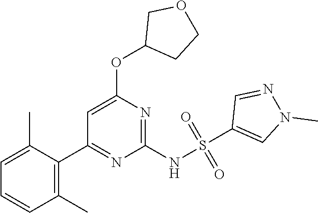 [SMILES: Cc1cccc(C)c1-c1cc(OC2CCOC2)nc(NS(=O)(=O)c2cnn(C)c2)n1] | 1.24 | 429.147 | 430 | A |
| 55 | 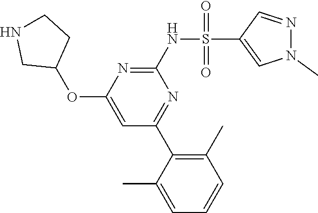 [SMILES: Cc1cccc(C)c1-c1cc(OC2CCNC2)nc(NS(=O)(=O)c2cnn(C)c2)n1] | 0.8 | 428.163 | 428 | A |
| 56 | 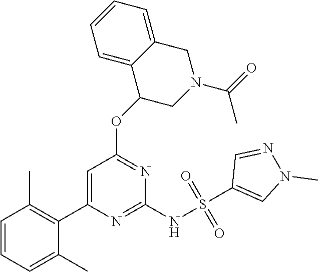 [SMILES: CC(=O)N1Cc2ccccc2C(Oc2cc(-c3c(C)cccc3C)nc(NS(=O)(=O)c3cnn(C)c3)n2)C1] | 1.36 | 532.189 | 533.3 | A |

| Compound | |
|---|---|
| number | NMR |
| 33 | ¹H NMR (400 MHz, DMSO-d₆) δ 13.75 (s, 1H), 8.32-8.21 (m, 3H), 7.78 |
| | (d, J = 0.7 Hz, 1H), 7.31-7.21 (m, 3H), 7.13 (d, J = 7.6 Hz, 2H), 6.39 (s, |
| | 1H), 5.23 (tt, J = 7.6, 3.9 Hz, 1H), 4.01-3.89 (m, 2H), 3.87 (s, 3H), 3.68 |
| | (ddd, J = 13.6, 8.2, 3.5 Hz, 2H), 2.14 (ddt, J = 13.8, 7.5, 3.8 Hz, 2H), 2.03 |
| | (s, 6H), 1.84-1.70 (m, 2H). |
| 50 | ¹H NMR (400 MHz, DMSO-d₆) δ 8.95 (d, J = 41.5 Hz, 2H), 8.26 (s, 1H), |
| | 7.75 (s, 1H), 7.29-7.20 (m, 1H), 7.13 (d, J = 7.6 Hz, 2H), 6.37 (s, 1H), 5.20 |
| | (s, 1H), 3.86 (s, 3H), 3.15 (s, 4H), 2.25 (d, J = 8.6 Hz, 1H), 2.02 (s, 8H), |
| | 1.94-1.84 (m, 2H), 1.78 (s, 1H). |
| 51 | ¹H NMR (400 MHz, DMSO-d₆) δ 8.25 (s, 1H), 7.72 (s, 1H), 7.24 (t, J = 7.6 |
| | Hz, 1H), 7.12 (d, J = 7.6 Hz, 2H), 6.33 (s, 1H), 4.90 (s, 1H), 4.15 (s, 1H), |
| | 3.84 (s, 3H), 3.78 (s, 1H), 3.24 (s, 1H), 2.91 (s, 1H), 2.03 (s, 6H), 1.85 (s, |
| | 2H), 1.68 (s, 1H), 1.45 (s, 1H), 1.24 (d, J = 54.2 Hz, 9H). |
| 52 | ¹H NMR (400 MHz, DMSO-d₆) δ 9.24 (s, 1H), 8.74 (s, 1H), 8.30 (s, 1H), |
| | 7.77 (s, 1H), 7.28-7.21 (m, 1H), 7.14 (d, J = 7.6 Hz, 2H), 6.36 (s, 1H), 5.21 |
| | (s, 1H), 3.87 (s, 3H), 3.38 (s, 3H), 3.11 (s, 1H), 3.01 (s, 1H), 2.03 (s, 6H), |
| | 1.86 (s, 3H), 1.75 (s, 1H). |
| 53 | ¹H NMR (400 MHz, DMSO-d₆) δ 8.25 (s, 1H), 7.74 (s, 1H), 7.28-7.21 (m, |
| | 1H), 7.13 (d, J = 7.6 Hz, 2H), 6.36 (s, 1H), 5.08 (s, 1H), 3.86 (s, 3H), 3.20 |
| | (s, 3H), 2.03 (s, 6H), 1.96 (s, 2H), 1.56 (s, 3H), 1.42 (s, 9H). |
| 54 | ¹H NMR (400 MHz, DMSO-d₆) δ 8.26 (s, 1H), 7.75 (s, 1H), 7.28-7.21 (m, |
| | 1H), 7.19-7.08 (m, 2H), 6.39 (s, 1H), 5.45 (s, 1H), 3.89 (dt, J = 10.5, 5.3 |
| | Hz, 2H), 3.85 (s, 3H), 3.78 (ddd, J = 17.3, 12.2, 8.8 Hz, 3H), 2.28 (dd, |
| | J = 13.2, 7.1 Hz, 1H), 2.02 (s, 6H), 1.97 (s, 1H). |
| 55 | ¹H NMR (400 MHz, DMSO-d₆) δ 9.39 (s, 1H), 9.27 (s, 1H), 8.32 (s, 1H), |
| | 7.79 (s, 1H), 7.30-7.22 (m, 1H), 7.14 (d, J = 7.6 Hz, 2H), 6.39 (s, 1H), 5.47 |
| | (s, 1H), 3.87 (s, 3H), 3.60 (s, 3H), 3.30 (s, 4H), 2.34 (s, 1H), 2.17 (s, 1H), |
| | 2.02 (s, 6H). |

### Example 12: Preparation of Compound 57

### Step 1: N-[4-(3-Aminopyrrolidin-1-yl)-6-(2,6-dimethylphenyl)pyrimidin-2-yl]-1 -methyl-pyrazole-4-sulfonamide (Compound 57)

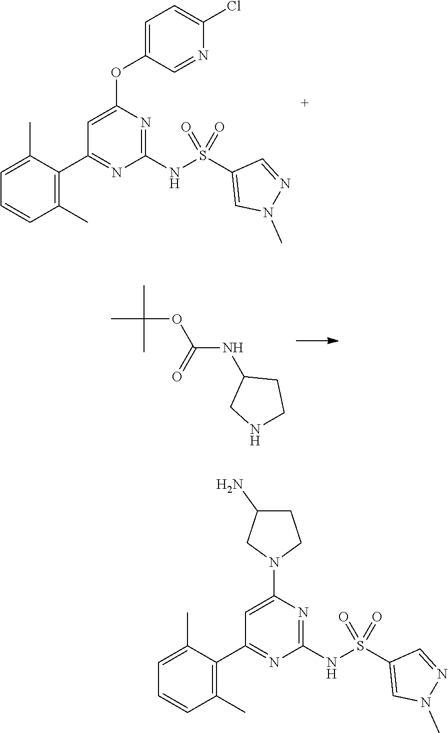 [SMILES: Cc1cccc(C)c1-c1cc(Oc2ccc(Cl)nc2)nc(NS(=O)(=O)c2cnn(C)c2)n1 ; CC(C)(C)OC(=O)NC1CCNC1 ; Cc1cccc(C)c1-c1cc(N2CCC(N)C2)nc(NS(=O)(=O)c2cnn(C)c2)n1]

N-[4-[(6-chloro-3-pyridyl)oxy]-6-(2,6-dimethylphenyl)pyrimidin-2-yl]-1-methyl-pyrazole-4-sulfonamide (approximately 29.99 mg, 0.06369 mmol), tert-butyl N-pyrrolidin-3-ylcarbamate (approximately 23.73 mg, 0.1274 mmol) and DIPEA (approximately 41.15 mg, 55.46 μL, 0.3184 mmol) were dissolved in NMP (0.5 mL) and heated to 130° C. in the microwave for 30 mins. The reaction mixture was filtered and purified by reverse phase HPLC (HCl modifier, 10-60% ACN-H₂O) to give tert-butyl N-[1-[6-(2,6-dimethylphenyl)-2-[(1-methylpyrazol-4-yl)sulfonylamino]pyrimidin-4-yl]pyrrolidin-3-yl]carbamate. ¹H NMR (400 MHz, DMSO-d₆) δ 8.16 (d, J=17.2 Hz, 1H), 7.72 (d, J=13.3 Hz, 1H), 7.22 (d, J=46.0 Hz, 4H), 6.02 (s, 1H), 4.15 (s, 1H), 3.86 (s, 3H), 3.79-3.21 (m, 5H), 2.14 (s, 8H), 1.99-1.83 (m, 1H), 1.41 (d, J=15.8 Hz, 9H). ESI-MS m/z calc. 527.23145, found 528.0 (M+1)+; Retention time: minutes (LC method A).

tert-Butyl N-[1-[6-(2,6-dimethylphenyl)-2-[(1-methylpyrazol-4-yl)sulfonylamino]pyrimidin-4-yl]pyrrolidin-3-yl]carbamate was stirred at room temperature for 30 min in a 1:1 TFA/DCM solution. The reaction mixture was concentrated and the residue was dissolved in DMSO, filtered and purified on reverse phase HPLC (HCl modifier, 1-50% ACN-H₂O) to give N-[4-(3-aminopyrrolidin-1-yl)-6-(2,6-dimethylphenyl)pyrimidin-2-yl]-1-methyl-pyrazole-4-sulfonamide (16.4 mg, 60%). ¹H NMR (400 MHz, DMSO-d₆) δ 8.44 (s, 2H), 8.38 (s, 1H), 8.17 (s, 1H), 7.72 (s, 1H), 7.27 (t, J=7.6 Hz, 1H), 7.15 (d, J=7.6 Hz, 2H), 6.04 (d, J=2.5 Hz, 1H), 3.90 (s, 3H), 3.73 (d, J=4.1 Hz, 3H), 3.63 (d, J=10.6 Hz, 1H), 3.54 (d, J=9.4 Hz, 1H), 2.31 (dd, J=14.1, 7.1 Hz, 1H), 2.13 (s, 7H). ESI-MS m/z calc. 427.17905, found 428.0 (M+1)+; Retention time: 0.65 minutes (LC method A).

### Example 13: Preparation of Compound 58

### Step 1: N-[4-(2,6-Dimethylphenyl)-6-indol-1-yl-pyrimidin-2-yl]-1-methyl-pyrazole-4-sulfonamide (Compound 58)

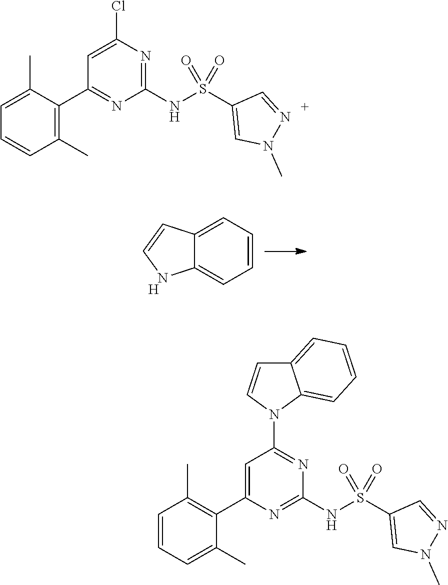

A solution of N-[4-chloro-6-(2,6-dimethylphenyl)pyrimidin-2-yl]-1-methyl-pyrazole-4-sulfonamide (40 mg, 0.1037 mmol) and indole (60 mg, 0.5122 mmol) in NMP (0.5 mL) was stirred at 80° C. for 2 hours. The reaction mixture was diluted with MeOH, filtered and purification by HPLC (1-99% ACN in water (HCl modifier)) gave N-[4-(2,6-dimethylphenyl)-6-indol-1-yl-pyrimidin-2-yl]-1-methyl-pyrazole-4-sulfonamide (21.8 mg, 46%) as an off-white solid. ESI-MS m/z calc. 458.1525, found 459.3 (M+1)⁺; Retention time: 1.71 minutes (LC method A).

### Example 14: Preparation of Compound 59

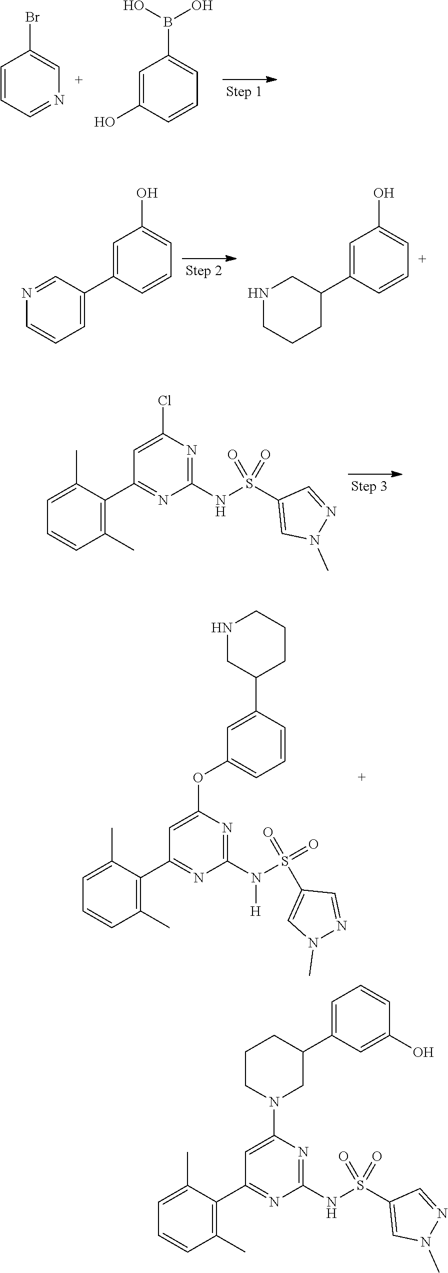

### Step 1: 3-(3-Pyridyl)phenol

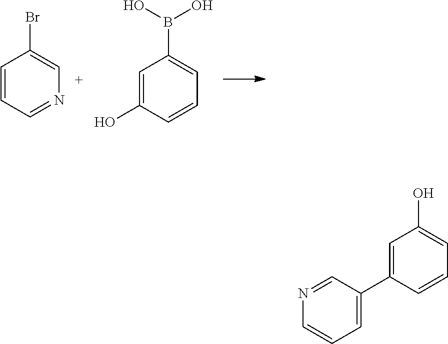

Tetrakis(triphenylphosphine)palladium(0) (838 mg, 0.725 mmol) was added to a solution of (3-hydroxyphenyl)boronic acid (10.02 g, 72.65 mmol), 3-bromopyridine (7 mL, 72.66 mmol) and sodium carbonate (15.37 g, 145.0 mmol) in mixture of tetrahydrofuran (140.0 mL), water (70.00 mL) and methanol (35.00 mL). The reaction mixture was bubbled with nitrogen for 5 minutes and heated at reflux for 2 hours followed by stirring at room temperature overnight. Reaction mixture was diluted with water (100 mL) and extracted with EtOAc (3×100 mL). Organic layers were combined, dried over sodium sulfate and concentrated under reduced pressure. (15.39 g of orange gum) The residue was purified by silica gel chromatography using 0% to 6% of methanol in dichloromethane to afford 3-(3-pyridyl)phenol (6.31 g, 51%) as yellow solid. ¹H NMR (300 MHz, DMSO-d₆) δ 6.77-6.85 (m, 1H), 7.04 (s, 1H), 7.10 (d, J=7.6 Hz, 1H), 7.23-7.34 (m, 1H), 7.45 (dd, J=7.9, 5.0 Hz, 1H), 7.98 (d, J=7.9 Hz, 1H), 8.54 (d, J=4.7 Hz, 1H), 8.77-8.83 (m, 1H), 9.61 (s, 1H). ESI-MS m/z calc. 171.06842, found 172.2 (M+1)⁺; Retention time: 0.7 minutes (LC method C).

### Step 2: 3-(3-Piperidyl)phenol

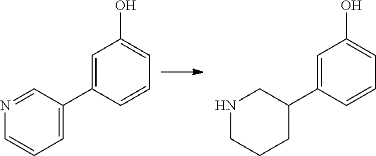 [SMILES: Oc1cccc(-c2cccnc2)c1 ; Oc1cccc(C2CCCNC2)c1]

Platinum oxide (837.0 mg, 3.686 mmol) was added to a solution of 3-(3-pyridyl)phenol (6.31 g, 36.86 mmol) in methanol (150 mL) and concentrated HCl (6 mL). Reaction mixture was placed under 50 PSI of hydrogen for 48 hours (16 hours with stirring). Reaction mixture was filtrated over Celite, washed with methanol and concentrated under reduced pressure to afford 3-(3-piperidyl)phenol (hydrochloride salt) (9.66 g, 123%) as yellow oil. ESI-MS m/z calc. 177.11537, found 178.2 (M+1)+; Retention time: 0.52 minutes (LC method C).

### Step 3: N-[4-(2,6-Dimethylphenyl)-6-[3-(3-hydroxyphenyl)-1-piperidyl]pyrimidin-2-yl]-1-methyl-pyrazole-4-sulfonamide (Compound 59)

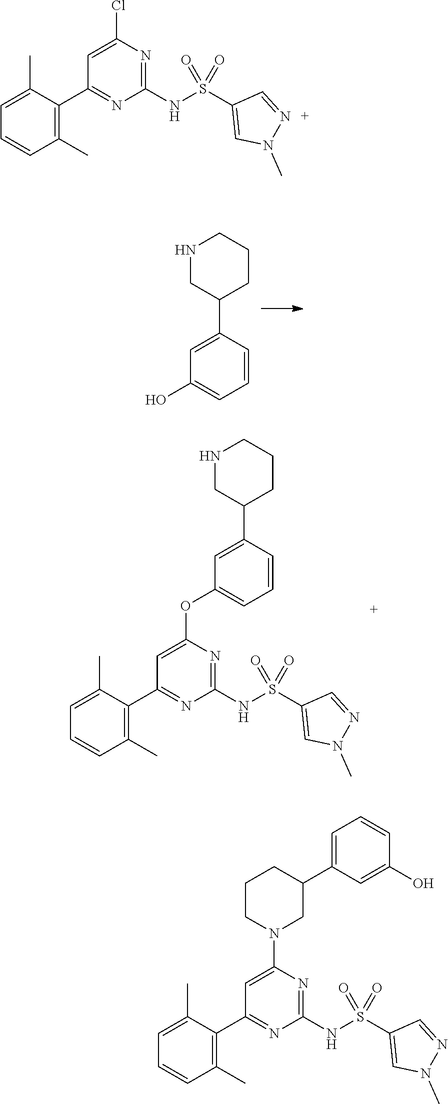

An NMP (0.8 mL) solution of 3-(3-piperidyl)phenol (51.2 mg, 0.289 mmol), Cs₂CO₃ (approximately 271 mg, 0.832 mmol) and N-[4-chloro-6-(2,6-dimethylphenyl)pyrimidin-2-yl]-1 -methyl-pyrazole-4-sulfonamide (50.7 mg, 0.1342 mmol) was heated to 110° C. for 20 hours and then cooled to room temperature. The solution was filtered and the resulting residue was dissolved in 0.7 mL DMSO, and purified by reverse phase chromatography using a 15 minute gradient of 20% MeCN in water to 80% MeCN to give N-[4-(2,6-dimethylphenyl)-6-[3-(3-piperidyl)phenoxy]pyrimidin-2-yl]-1-methyl-pyrazole-4-sulfonamide (5.1 mg, 3%) ESI-MS m z calc. 518.21, found 519.55 (M+1)+; Retention time: 1.24 minutes (LC method A). and N-[4-(2,6-dimethylphenyl)-6-[3-(3-hydroxyphenyl)-1-piperidyl]pyrimidin-2-yl]-1-methyl-pyrazole-4-sulfonamide (5.6 mg, 4%) ESI-MS m/z calc. 518.21, found 519.55 (M+1)+; Retention time: 1.39 minutes (LC method A).

### Example 15: Characterization of Compounds 60-66

The compounds in the following tables were prepared in a manner analogous to that described above using commercially available reagents and intermediates described herein.

| Compound | | LCMS | Calc. | | LCMS |
|---|---|---|---|---|---|
| number | Structure | Rt (min) | mass | M + 1 | Method |
| 60 | 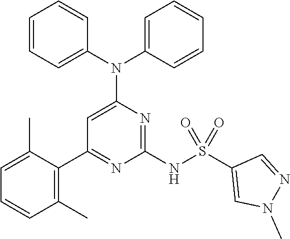 [SMILES: Cc1cccc(C)c1-c1cc(N(c2ccccc2)c2ccccc2)nc(NS(=O)(=O)c2cnn(C)c2)n1] | 1.65 | 510.184 | 511.4 | A |
| 61 | 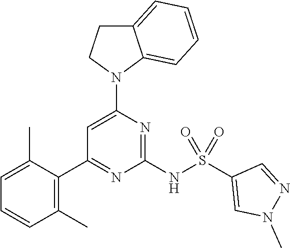 [SMILES: Cc1cccc(C)c1-c1cc(N2CCc3ccccc32)nc(NS(=O)(=O)c2cnn(C)c2)n1] | 1.42 | 460.168 | 461.3 | A |
| 62 | 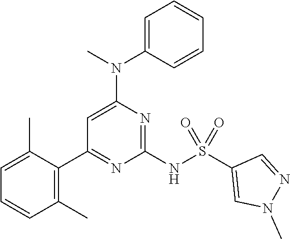 [SMILES: Cc1cccc(C)c1-c1cc(N(C)c2ccccc2)nc(NS(=O)(=O)c2cnn(C)c2)n1] | 1.4 | 448.168 | 449.3 | A |
| 63 | 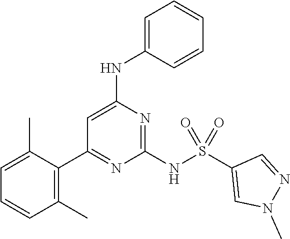 [SMILES: Cc1cccc(C)c1-c1cc(Nc2ccccc2)nc(NS(=O)(=O)c2cnn(C)c2)n1] | 1.22 | 434.152 | 435.3 | A |
| 64 | 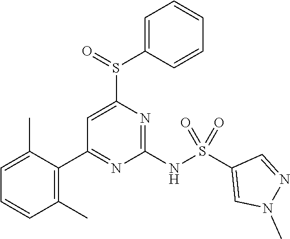 [SMILES: Cc1cccc(C)c1-c1cc(S(=O)c2ccccc2)nc(NS(=O)(=O)c2cnn(C)c2)n1] | 1.45 | 467.109 | 468.1 | A |
| 65 | 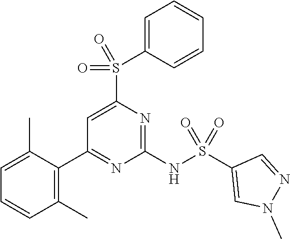 [SMILES: Cc1cccc(C)c1-c1cc(S(=O)(=O)c2ccccc2)nc(NS(=O)(=O)c2cnn(C)c2)n1] | 1.59 | 483.103 | 484.1 | A |
| 66 | 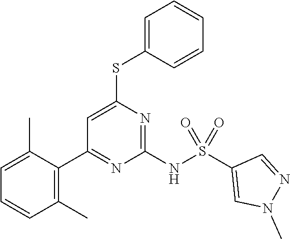 [SMILES: Cc1cccc(C)c1-c1cc(Sc2ccccc2)nc(NS(=O)(=O)c2cnn(C)c2)n1] | 1.77 | 451.114 | 452.3 | A |

### Example 16: Preparation of Compound 67

### Step 1: 5-[6-(2,6-Dimethylphenyl)-2-[(1-methylpyrazol-4-yl)sulfonylamino]pyrimidin-4-yl]oxy-N-methyl-pyridine-2-carboxamide (Compound 67)

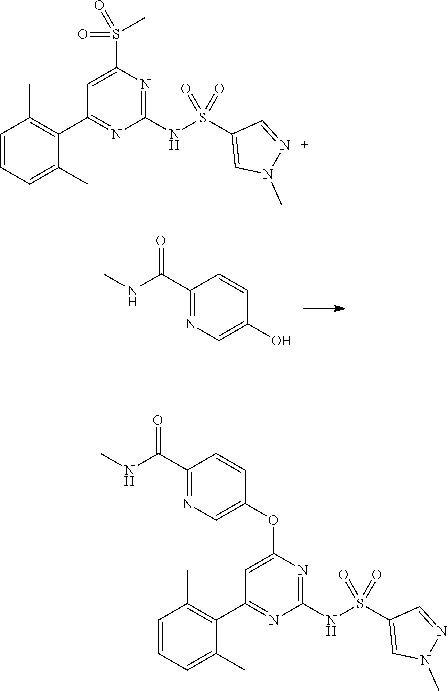

A suspension of N-[4-(2,6-dimethylphenyl)-6-methylsulfonyl-pyrimidin-2-yl]-1 -methyl-pyrazole-4-sulfonamide (78 mg, 0.1851 mmol), 5-hydroxy-N-methyl-pyridine-2-carboxamide (83 mg, 0.5455 mmol) and K₂CO₃ (120 mg, 0.8683 mmol) in NMP (1 mL) was stirred at 120° C. for 3 hours. The reaction mixture was diluted with water and the pH was adjusted to ˜4 with conc. HCl, then extracted with EtOAc (3×). Organics were combined, evaporated and the material was purified by column chromatography (12 g silica; 10-100% EtOAc in hexanes) to give 5-[6-(2,6-dimethylphenyl)-2-[(1-methylpyrazol-4-yl)sulfonylamino]pyrimidin-4-yl]oxy-N-methyl-pyridine-2-carboxamide (34.8 mg, 38%) as a white solid. ESI-MS m/z calc. 493.15323, found 494.2 (M+1)+; Retention time: 1.34 minutes (LC method A). ¹H NMR (400 MHz, DMSO-d₆) δ 11.71 (s, 1H), 8.80 (d, J=4.8 Hz, 1H), 8.68(s, 1H), 8.15 (d, J=8.6 Hz, 1H), 8.04-7.95 (m, 1H), 7.78 (s, 1H), 7.33-7.22 (m, 2H), 7.15 (d, J=7.6 Hz, 2H), 6.82 (s, 1H), 3.74 (s, 3H), 2.85 (d, J=4.8 Hz, 3H), 2.05 (s, 6H).

### Example 17: Preparation of Compound 68

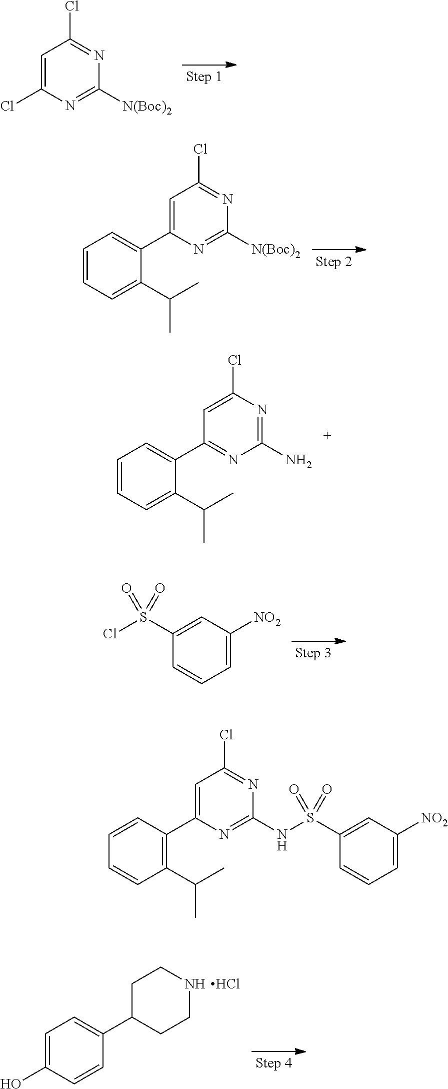

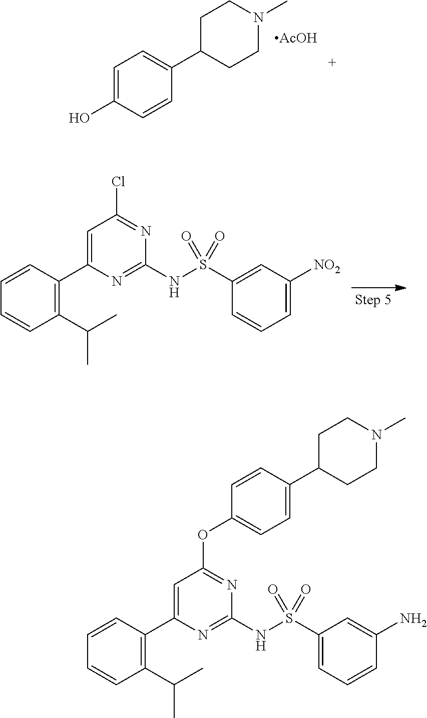 [SMILES: "·AcOH" ; CN1CCC(c2ccc(O)cc2)CC1 ; CC(C)c1ccccc1-c1cc(Cl)nc(NS(=O)(=O)c2cccc([N+](=O)[O-])c2)n1 ; CC(C)c1ccccc1-c1cc(Oc2ccc(C3CCN(C)CC3)cc2)nc(NS(=O)(=O)c2cccc(N)c2)n1]

### Step 1: tert-Butyl N-tert-butoxycarbonyl-N-[4-chloro-6-(2-isopropylphenyl)pyrimidin-2-yl]carbamate

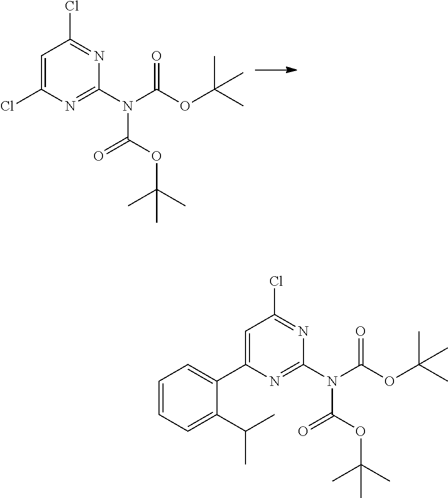 [SMILES: CC(C)(C)OC(=O)N(C(=O)OC(C)(C)C)c1nc(Cl)cc(Cl)n1 ; CC(C)c1ccccc1-c1cc(Cl)nc(N(C(=O)OC(C)(C)C)C(=O)OC(C)(C)C)n1]

A solution of tert-butyl N-[(tert-butoxy)carbonyl]-N-(4,6-dichloropyrimidin-2-yl)carbamate (128.5 g, 0.353 mol), 2-isopropylphenylboronic acid (57.86 g, 0.353 mol) and cesium carbonate (288 g, 0.883 mol) in 1,2-dimethoxyethane (1.0 L) and water (250 mL) was purged with nitrogen, and then 1,1′-bis(diphenylphosphino)ferrocene dichloropalladium(II) (12.9 g, 0.0177 mol) was added. The reaction was stirred at 80° C. for 1 hour. Two layers were separated, and the aqueous layer was extracted with ethyl acetate (2×500 mL). The combined organic layers were washed with brine (500 mL), dried over anhydrous sodium sulfate and concentrated under vacuum. The crude product was purified by recrystallization with methanol (300 mL) to furnish tert-butyl N-[(tert-butoxy)carbonyl]-N-{4-chloro-6-[2-(propan-2-yl)phenyl]pyrimidin-2-yl}carbamate (95.63 g, 60%) as a beige solid. ESI-MS m/z: calc. 447.2, found 448.1 (M1). Retention time: 7.07 minutes. ¹H NMR (250 MHz, CDCl₃) δ 7.46 (m, 2H), 7.35 (s, 1H), 7.32-7.23 (m, 2H), 3.23 (m, 1H), 1.45 (s, 18H), 1.23 (d, J=6.8 Hz, 6H).

### Step 2: 4-Chloro-6-(2-isopropylphenyl)pyrimidin-2-amine

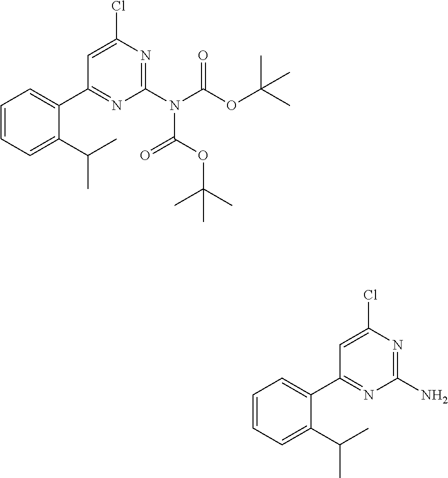 [SMILES: CC(C)c1ccccc1-c1cc(Cl)nc(N(C(=O)OC(C)(C)C)C(=O)OC(C)(C)C)n1 ; CC(C)c1ccccc1-c1cc(Cl)nc(N)n1]

A 2 L round bottom flask was charged with a solution of tert-butyl N-[(tert-butoxy)carbonyl]-N-{4-chloro-6-[2-(propan-2-yl)phenyl]pyrimidin-2-yl}carbamate (95.63 g, 0.213 mol) in dichloromethane (1.0 L). A 4 M hydrogen chloride solution in dioxane (400 mL) was added to the reaction mixture at 0° C. The reaction was stirred at room temperature for 5 hours. Dichloromethane was removed under vacuum. Diethyl ether (400 mL) was added to the reaction mixture to precipitate out the hydrochloride salt of the desired product. The mixture was stirred in an ice bath for 20 minutes. The solid was then collected by suction filtration and dried under vacuum to furnish 4-chloro-6-(2-isopropylphenyl)pyrimidin-2-amine hydrochloride (53.36 g, 88%) as an off-white solid. The salt was dissolved into a mixture of dichloromethane (500 mL) and saturated sodium bicarbonate aqueous solution (500 mL), and it was stirred at room temperature for 1 hour. Two layers were separated. The aqueous layer was extracted with dichloromethane (2×500 mL). The combined organic layers were washed with brine (500 mL), dried over anhydrous magnesium sulfate and concentrated under vacuum. The solid was then triturated with hexane and collected by suction filtration and dried under vacuum to furnish 4-chloro-6-(2-isopropylphenyl)pyrimidin-2-amine (44.64 g, 85%) as an off-white solid. ESI-MS m/z calc. 247.08763, found 248.1 (M+1)⁺; Retention time: 4.73 minutes. ¹H NMR (250 MHz, CDCl₃) δ 7.42 (m, 2H), 7.25 (m, 2H), 6.73 (s, 1H), 5.39 (br, 2H), 3.19 (m, 1H), 1.23 (d, J=6.8 Hz, 6H).

### Step 3: N-[4-Chloro-6-(2-isopropylphenyl)pyrimidin-2-yl]-3-nitro-benzenesulfonamide

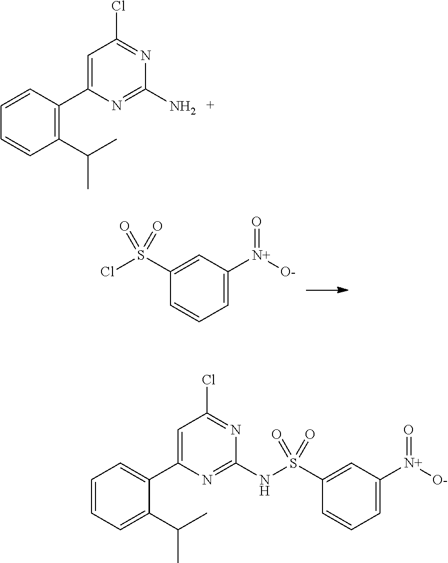

In a dry glass vial was added NaH (800.2 mg, 20.01 mmol) and THE (11 mL) and the white suspension was cooled to 0° C. with an ice-water bath and purged under nitrogen. 4-Chloro-6-(2-isopropylphenyl)pyrimidin-2-amine (1.2402 g, 5.0064 mmol) was dissolved in THE (3 mL) and added dropwise to the reaction mixture via syringe over 15 minutes. The mixture was then warmed to room temperature for 30 minutes. The reaction was cooled with ice-water bath to 0° C. and then 3-nitrobenzenesulfonyl chloride (2.2085 g, 9.9653 mmol) dissolved in THE (5 mL) was added. The reaction was stirred for 5 minutes and quenched with HCl (23 mL of 1 M, 23.000 mmol). Ethyl acetate (25 mL) was added. The organic layer was separated and dried with anhydrous sodium sulfate, filtered, and concentrated in vacuo. The residue was loaded onto a 40 g silica gel column and purified by column chromatography using a 19 minutes gradient of 0-70% ethyl acetate/hexanes to give N-[4-chloro-6-(2-isopropylphenyl)pyrimidin-2-yl]-3-nitro-benzenesulfonamide (1.89 g, 87%). ¹H NMR (400 MHz, DMSO-d₆) δ 8.76-8.65 (m, 1H), 8.49 (ddd, J=8.3, 2.4, 1.0 Hz, 1H), 8.34 (dt, J=8.0, 1.2 Hz, 1H), 7.94-7.82 (m, 1H), 7.52 -7.41 (m, 2H), 7.36 (s, 1H), 7.25 (ddd, J=8.4, 5.1, 3.5 Hz, 1H), 7.13 (dd, J=7.4, 1.1 Hz, 1H), 3.16-3.02 (m, 1H), 1.08 (d, J=6.8 Hz, 6H). ESI-MS m/z calc. 432.06592, found 433.38 (M+1)⁺; Retention time: 0.77 minutes (LC method D).

### Step 4: 4-(1-Methyl-piperidin-4-yl)-phenol

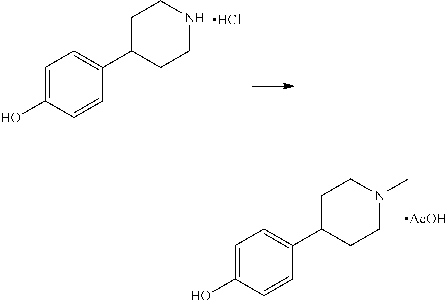

To a stirring solution of 4-(piperdin-4-yl) phenol hydrochloride (10.58 g, 49.6 mmol), 37 wt % solution of formaldehyde in water (20.9 mL, 282.2 mmol), N,N-diisopropylethylamine (8.68 mL, 50.1 mmol) and tetrahydrofuran (170 mL) cooled in an ice bath was added sodium triacetoxyborohydride (21.2 g, 100.2 mmol) portion wise. The ice bath was removed and after 30 minutes the reaction was concentrated to a gel. The crude was quenched with saturated aqueous sodium bicarbonate (150 mL) and extracted with ethyl acetate (150 mL×3) and 9:1 mixture of chloroform: isopropanol (150 mL×3). The aqueous, still containing product was evaporated. All organics were combined with the evaporated aqueous layer and dissolved in methanol (50 mL) along with 15 g silica gel and 15 mL N,N-diisopropylethylamine and evaporated. The dry loaded crude was purified on silica gel using 0-30% methanol in chloroform (0.5% trimethylamine modifier) to give 4-(1-methyl-piperidin-4-yl)-phenol acetic acid salt (7.38 g, 74%) as a beige solid. ESI-MS m/z calc. 191.3 found 192.0 (M+1). Retention time: 1.12 minutes. ¹H NMR (250 MHz, DMSO-d₆) δ 0.95-1.05 (m, 1H) 1.61 (m, 3H) 1.91 (s, 3H) 1.95 -2.05 (m, 2H) 2.20 (s, 3H) 2.26-2.41 (m, 1H) 2.86 (d, J=10.88 Hz, 2H) 6.61-6.76 (m, 2H) 6.95-7.09 (m, 2H)

### Step 5: 3-Amino-N-[4-(2-isopropylphenyl)-6-[4-(1-methyl-4-piperidyl)phenoxy]pyrimidin-2-yl]benzenesulfonamide (Compound 68)

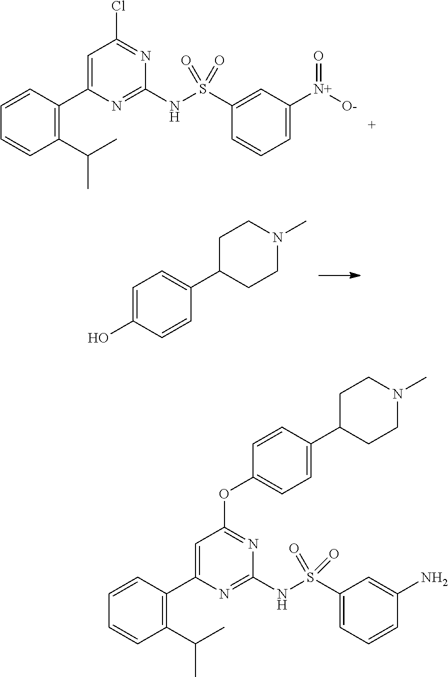 [SMILES: CC(C)c1ccccc1-c1cc(Cl)nc(NS(=O)(=O)c2cccc([N+](=O)[O-])c2)n1 ; CN1CCC(c2ccc(O)cc2)CC1 ; CC(C)c1ccccc1-c1cc(Oc2ccc(C3CCN(C)CC3)cc2)nc(NS(=O)(=O)c2cccc(N)c2)n1]

A NMP (0.8 mL) mixture of 4-(1-methyl-4-piperidyl)phenol (acetate salt) (approximately 43 mg, 0.17 mmol), N-[4-chloro-6-(2-isopropylphenyl)pyrimidin-2-yl]-3-nitro-benzenesulfonamide (25 mg, 0.058 mmol), and Cs₂CO₃ (approximately 75 mg, 0.23 mmol) was heated to 110° C. for 16 hours and then cooled to room temperature. The mixture was diluted with water (0.5 mL) and ethyl acetate (1.5 mL) was added and the organic layer was separated, dried with anhydrous sodium sulfate, filtered, and concentrated in vacuo. The resulting residue was taken up in EtOH (0.9 mL) and HCl (0.1 mL of 0.5 M) was added and the mixture heated to 50° C. for 10 minutes. The solution was filtered, and the resulting residue was dissolved in 0.8 mL MeOH, and purified by reverse phase chromatography using a 15 minutes gradient of 1% MeCN in water to 99% MeCN to give 3-amino-N-[4-(2-isopropylphenyl)-6-[4-(1-methyl-4-piperidyl)phenoxy]pyrimidin-2-yl]benzenesulfonamide (dihydrochloride salt) (5.6 mg, 15%). ESI-MS m/z calc. 557.24603, found 558.63 (M+1)⁺; Retention time: 1.43 minutes (LC method A). ¹H NMR (400 MHz, DMSO-d₆) δ 7.55-7.32 (m, 4H), 7.29-7.12 (m, 4H), 7.05-6.85 (m, 2H), 6.78-6.60 (m, 1H), 6.60-6.39 (m, 2H), 5.50 (s, 2H), 3.18-2.93 (m, 4H), 2.93-2.81 (m, 1H), 2.75 (s, 3H), 2.17-1.81 (m, 4H), 1.09 (d, J=6.6 Hz, 6H), 2H likely overlapped under water signal.

### Example 18: Preparation of Compound 69

### Step 1: 2-Chloro-3-(4-methylpiperazin-1-yl)phenol

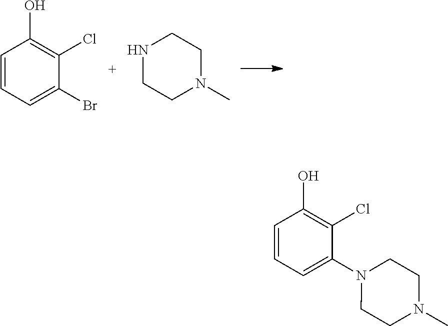

A heterogeneous mixture of 3-bromo-2-chloro-phenol (4.20 g, 20.25 mmol), 1-methylpiperazine (21.5 g, 214.7 mmol), chloro(2-di-t-butylphosphino-2′, 4′, 6′-tri-i-propyl-1,1′-biphenyl)[2-(2-aminoethyl)phenyl] palladium(II) [t-BuXPhos Palladacycle Gen. 1] (2.1 g, 3.058 mmol), and potassium tert-butoxide (4.8 g, 42.78 mmol) in dioxane (120 mL) was sonicated for 15 minutes and heated in a sealed vessel at 50° C. for 16 hours. The reaction mixture was acidified using acetic acid (3.4 mL, 59.79 mmol) then partitioned between DCM (100 mL) and water (100 mL). The organic layer was separated, and the aqueous layer was further extracted with DCM (4×). The combined organic phases were washed once with brine, dried using magnesium sulfate, filtered, and concentrated in vacuo. The crude residue was separated by flash column chromatography on silica gel (gradient: 1 to 10% methanol in dichloromethane) to afford 2-chloro-3-(4-methylpiperazin-1-yl)phenol (3.86 g, 84%) as a yellow solid. ESI-MS m z calc. 226.0873, found 227.12 (M+1)⁺; Retention time: 0.24 minutes (LC method D).

### Step 2: 3-Amino-N-[4-[2-chloro-3-(4-methylpiperazin-1-yl)phenoxy]-6-(2-isopropylphenyl)pyrimidin-2-yl]benzenesulfonamide (Compound 69)

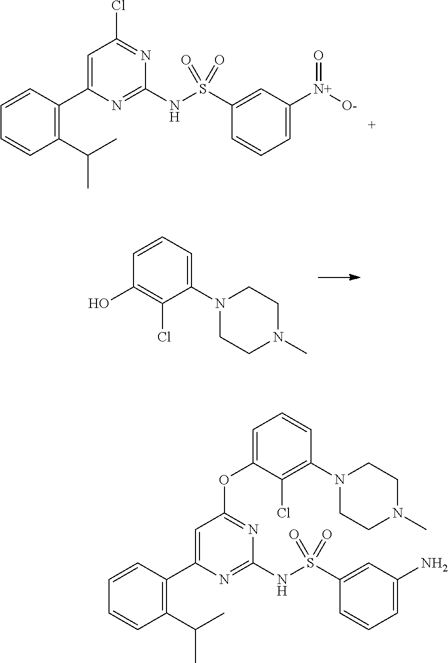 [SMILES: CC(C)c1ccccc1-c1cc(Cl)nc(NS(=O)(=O)c2cccc([N+](=O)[O-])c2)n1 ; CN1CCN(c2cccc(O)c2Cl)CC1 ; CC(C)c1ccccc1-c1cc(Oc2cccc(N3CCN(C)CC3)c2Cl)nc(NS(=O)(=O)c2cccc(N)c2)n1]

A NMP (0.8 mL) mixture of 2-chloro-3-(4-methylpiperazin-1-yl)phenol (approximately 39.29 mg, 0.1733 mmol), N-[4-chloro-6-(2-isopropylphenyl)pyrimidin-2-yl]-3-nitro-benzenesulfonamide (25 mg, 0.05775 mmol), and Cs₂CO₃ (approximately 75.26 mg, 0.2310 mmol) was heated to 110° C. for 16 hours and then cooled to room temperature. The mixture was diluted with water (0.5 mL) and ethyl acetate (1.5 mL) was added and the organic layer was separated, dried with anhydrous sodium sulfate, filtered, and concentrated in vacuo. The resulting residue was taken up in EtOH (0.9 mL) and HCl (0.1 mL of 0.5 M) was added and the mixture was heated to 50° C. for 10 minutes. The solution was filtered and the resulting residues dissolved in 0.8 mL MeOH, and purified by reverse phase chromatography using a 15minute gradient of 1% MeCN in water to 99% MeCN to give 3-amino-N-[4-[2-chloro-3-(4-methylpiperazin-1-yl)phenoxy]-6-(2-isopropylphenyl)pyrimidin-2-yl]benzenesulfonamide (dihydrochloride salt) (7.2 mg, 19%). ESI-MS m/z calc. 592.20233, found 593.53 (M+1)⁺; Retention time: 1.49 minutes (LC method A).

### Example 19: Preparation of Compound 70

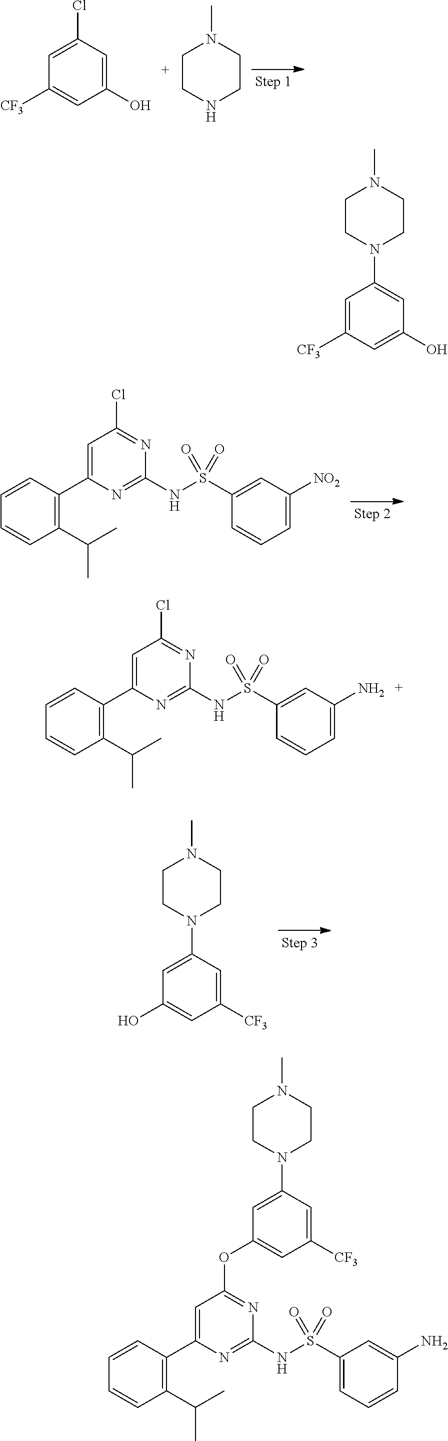

### Step 1: 3-(4-Methylpiperazin-1-yl)-5-(trifluoromethyl)phenol

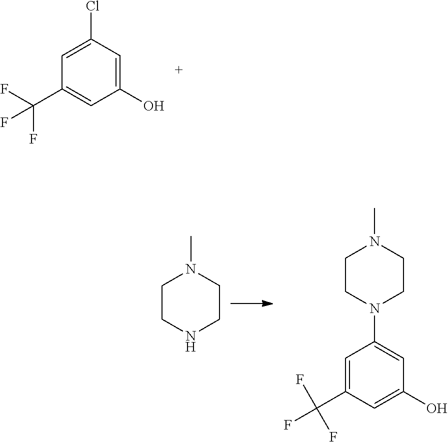

A dioxane (1 mL) mixture of 3-chloro-5-(trifluoromethyl)phenol (50 mg, 0.2544 mmol), 1-methylpiperazine (51.2 mg, 0.5112 mmol), chloro(2-di-t-butylphosphino-2′, 4′, 6′-tri-i-propyl-1,1′-biphenyl)[2-(2-aminoethyl)phenyl] palladium(II) (t-BuXPhos Palladacycle Gen. 1) (approximately 16.57 mg, 0.02544 mmol), and sodium tert-butoxide (approximately 61.12 mg, 0.6360 mmol) was sparged with nitrogen and then stirred at 35° C. for 2 hours. The solution was filtered and the filtrate was dissolved in 0.8 mL MeOH, and purified by reverse phase chromatography using a 15 minute gradient of 1% MeCN in water to 50% MeCN (HCl modifier) to give 3-(4-methylpiperazin-1-yl)-5-(trifluoromethyl)phenol (hydrochloride salt) (48.4 mg, 64%). ¹H NMR (400 MHz, DMSO-d₆) δ 10.88 (s, 1H), 6.75-6.70 (m, 1H), 6.64-6.60 (m, 1H), 6.58-6.53 (m, 1H), 3.93-3.78 (m, 2H), 3.52-3.36 (m, 2H), 3.21-3.01 (m, 4H), 2.80 (d, J=4.7 Hz, 3H). ESI-MS m/z calc. 260.11365, found 261.3 (M+1)⁺; Retention time: 0.9 minutes (LC method A).

### Step 2: 3-Amino-N-[4-chloro-6-(2-isopropylphenyl)pyrimidin-2-yl]benzenesulfonamide

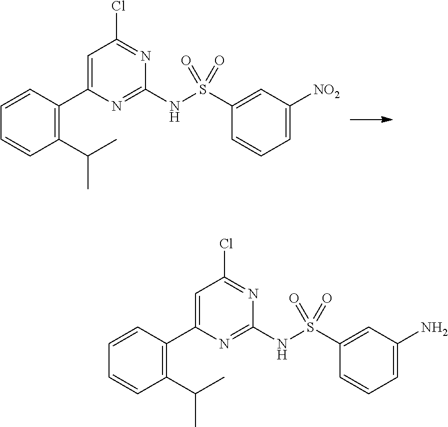 [SMILES: CC(C)c1ccccc1-c1cc(Cl)nc(NS(=O)(=O)c2cccc([N+](=O)[O-])c2)n1 ; CC(C)c1ccccc1-c1cc(Cl)nc(NS(=O)(=O)c2cccc(N)c2)n1]

A solution of N-[4-chloro-6-(2-isopropylphenyl)pyrimidin-2-yl]-3-nitro-benzenesulfonamide (231 mg, 0.5336 mmol), hydrochloric acid (130 μL of 37% w/v, 1.319 mmol) and iron (149 mg, 2.668 mmol) in ethanol (5.3 mL) was stirred at 23° C. for 16 hours. The reaction was diluted with diethyl ether, filtered, and concentrated onto silica gel. The crude impregnated silica was subjected to flash column chromatography (10 to 100% ethyl acetate in hexanes) to afford 3-amino-N-[4-chloro-6-(2-isopropylphenyl)pyrimidin-2-yl]benzenesulfonamide (214 mg, 94%) as a yellow solid. ESI-MS m/z calc. 402.09174, found 403.33 (M+1)⁺; Retention time: 0.68 minutes (LC method D).

### Step 3: 3-Amino-N-[4-(2-isopropylphenyl)-6-[3-(4-methylpiperazin-1-yl)-5-(trifluoromethyl)phenoxy]pyrimidin-2-yl]benzenesulfonamide (Compound 70)

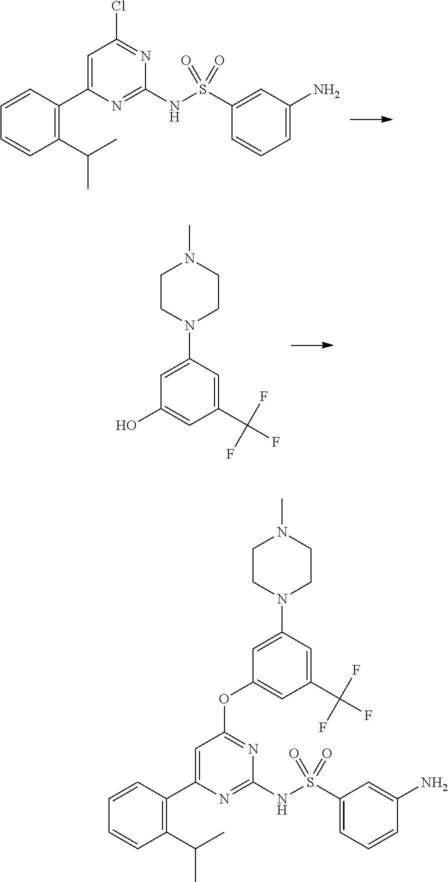 [SMILES: CC(C)c1ccccc1-c1cc(Cl)nc(NS(=O)(=O)c2cccc(N)c2)n1 ; CN1CCN(c2cc(O)cc(C(F)(F)F)c2)CC1 ; CC(C)c1ccccc1-c1cc(Oc2cc(N3CCN(C)CC3)cc(C(F)(F)F)c2)nc(NS(=O)(=O)c2cccc(N)c2)n1]

A NMP (0.5 mL) mixture of 3-(4-methylpiperazin-1-yl)-5-(trifluoromethyl)phenol (hydrochloride salt) (approximately 22 mg, 0.075 mmol), 3-amino-N-[4-chloro-6-(2-isopropylphenyl)pyrimidin-2-yl]benzenesulfonamide (10 mg, 0.025 mmol), and Cs₂CO₃ (approximately 49 mg, 0.15 mmol) was stirred at 110° C. for 16 hours and then cooled to room temperature. The solution was filtered and the resulting residue was dissolved in 0.8 mL MeOH, and purified by reverse phase chromatography using a 15 min gradient of 1% MeCN in water to 99% MeCN (HCl modifier) to give 3-amino-N-[4-(2-isopropylphenyl)-6-[3-(4-methylpiperazin-1-yl)-5-(trifluoromethyl)phenoxy]pyrimidin-2-yl]benzenesulfonamide (dihydrochloride salt) (2.2 mg, 13%). ESI-MS m/z calc. 626.2287, found 627.57 (M+1)⁺; Retention time: 1.64 minutes (LC method A).

### Example 20: Preparation of Compound 71

### Step 1: 3-Amino-N-[4-(2-isopropylphenyl)-6-(4-piperazin-1-ylphenoxy)pyrimidin-2-yl]benzenesulfonamide (Compound 71)

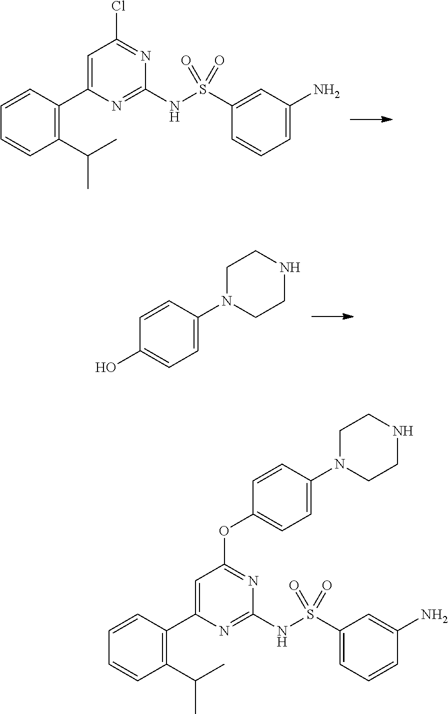 [SMILES: CC(C)c1ccccc1-c1cc(Cl)nc(NS(=O)(=O)c2cccc(N)c2)n1 ; Oc1ccc(N2CCNCC2)cc1 ; CC(C)c1ccccc1-c1cc(Oc2ccc(N3CCNCC3)cc2)nc(NS(=O)(=O)c2cccc(N)c2)n1]

A NMP (0.5 mL) mixture of 4-piperazin-1-ylphenol (approximately 19.91 mg, 0.1117 mmol), 3-amino-N-[4-chloro-6-(2-isopropylphenyl)pyrimidin-2-yl]benzenesulfonamide (15 mg, 0.03723 mmol), and Cs₂CO₃ (approximately 48.51 mg, 0.1489 mmol) was stirred at 110° C. for 16 hours and then cooled to room temperature. The solution was filtered and the resulting residue was dissolved in 0.8 mL MeOH, and purified by reverse phase chromatography using a 15 minute gradient of 1% MeCN in water to 99% MeCN (HCl modifier) to give 3-amino-N-[4-(2-isopropylphenyl)-6-(4-piperazin-1-ylphenoxy)pyrimidin-2-yl]benzenesulfonamide (dihydrochloride salt) (6.2 mg, 31%). ¹H NMR (400 MHz, DMSO-d₆) δ 9.17 (s, 2H), 7.48-7.40 (m, 2H), 7.31-7.21 (m, 2H), 7.21-7.08 (m, 6H), 7.05 (s, 1H), 6.88 (d, J=7.6 Hz, 1H), 6.51 (s, 1H), 4.26 (s, 1H), 3.40 (t, J=5.2 Hz, 4H), 3.24 (m, 5H), 3.09 (p, J=6.9 Hz, 1H), 1.10 (d, J=6.8 Hz, 6H). ESI-MS m/z calc. 544.22565, found 545.56 (M+1)⁺; Retention time: 1.39 minutes (LC method A).

### Example 21: Preparation of Compound 72

### Step 1: 3-Amino-N-[4-[3-(dimethylamino)phenoxy]-6-(2-isopropylphenyl)pyrimidin-2-yl]benzenesulfonamide (Compound 72)

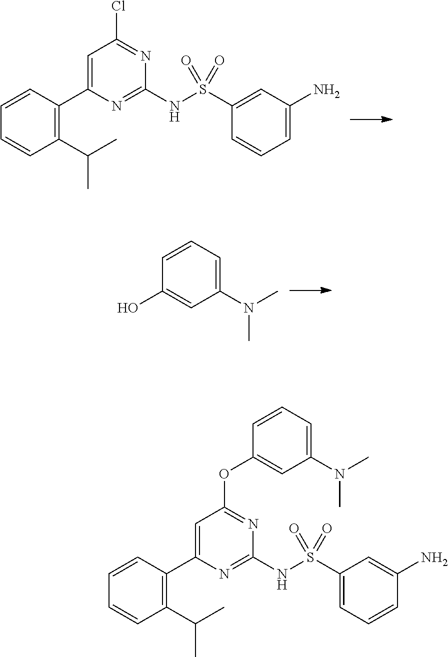

A NMP (0.5 mL) mixture of 3-(dimethylamino)phenol (approximately 15.32 mg, 0.1117 mmol), 3-amino-N-[4-chloro-6-(2-isopropylphenyl)pyrimidin-2-yl]benzenesulfonamide (15 mg, 0.03723 mmol), and Cs₂CO₃ (approximately 48.51 mg, 0.1489 mmol) was stirred at 110° C. for 16 hours and then cooled to room temperature. The solution was filtered and the resulting residue was dissolved in 0.8 mL MeOH, and purified by reverse phase chromatography using a 15 min gradient of 1% MeCN in water to 99% MeCN (HCl modifier) to give 3-amino-N-[4-[3-(dimethylamino)phenoxy]-6-(2-isopropylphenyl)pyrimidin-2-yl]benzenesulfonamide (hydrochloride salt). ESI-MS m/z calc. 503.1991, found 504.31 (M+1)⁺; Retention time: 1.58 minutes (LC method A).

### Example 22: Preparation of Compound 73

### Step 1: 2-Chloro-5-(4-methylpiperazin-1-yl)phenol

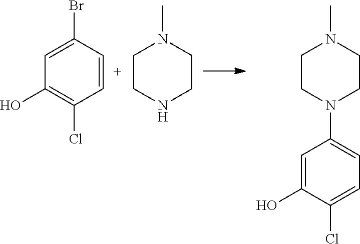

In a glass vial was 5-bromo-2-chloro-phenol (350 mg, 1.687 mmol), [2-(2-aminoethyl)phenyl]-chloro-palladium;ditert-butyl-[2-(2,4,6-triisopropylphenyl) phenyl]phosphane (XPhos Pd G1)(approximately 115.8 mg, 0.1687 mmol), 1-000 (approximately 1.690 g, 1.874 mL, 16.87 mmol), and dioxane (10 mL) and the mixture was sparged with nitrogen for 30 minutes and then solid sodium tert-butoxide (approximately 340.5 mg, 3.543 mmol) was added. The reaction was stirred under nitrogen for 15 minutes at room temperature and then poured into a saturated aqueous solution of ammonium chloride (25 mL) and dichloromethane (25 mL). pH of aqueous layer ˜8-9. The aqueous layer was extracted with dichloromethane (25 mL). The organic layers were combined and dried over anhydrous magnesium sulfate and then filtered and concentrated in vacuo. The mixture was chased with ethyl acetate (2×10 mL) to remove residual dioxane and DCM and then taken up in ethyl acetate (3.5 mL, 10 volumes) and heated to 80° C. to dissolve everything and then stirred and cooled to room temperature over 1 hour and stirred at room temperature for 16 hours. The wet cake was filtered and washed with ethyl acetate (0.5 mL) followed by diethyl ether (1 mL) to afford 2-chloro-5-(4-methylpiperazin-1-yl)phenol. ¹H NMR (400 MHz, Chloroform-d) δ 7.14 (d, J=8.9 Hz, 1H), 6.56 (d, J=2.8 Hz, 1H), 6.45 (dd, J=8.9, 2.9 Hz, 1H), 3.22-3.13 (m, 4H), 2.60-2.52 (m, 5H), 2.34 (s, 3H). ESI-MS m/z calc. 226.0873, found 227.29 (M+1)⁺; Retention time: 0.64 minutes (LC method A).

### Step 2: 3-Amino-N-[4-[2-chloro-5-(4-methylpiperazin-1-yl)phenoxy]-6-(2-isopropylphenyl)pyrimidin-2-yl]benzenesulfonamide (Compound 73)

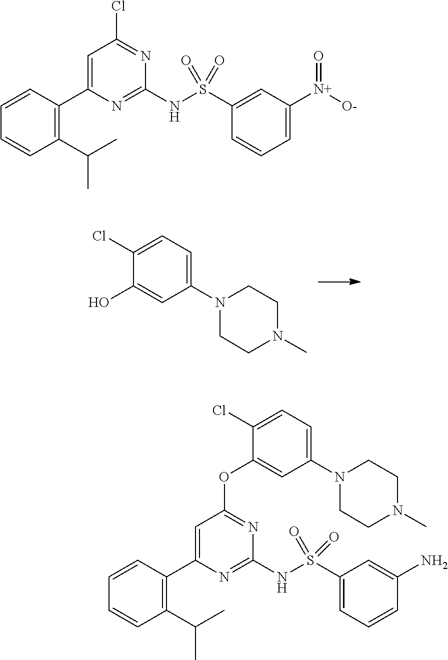 [SMILES: CC(C)c1ccccc1-c1cc(Cl)nc(NS(=O)(=O)c2cccc([N+](=O)[O-])c2)n1 ; CN1CCN(c2ccc(Cl)c(O)c2)CC1 ; CC(C)c1ccccc1-c1cc(Oc2cc(N3CCN(C)CC3)ccc2Cl)nc(NS(=O)(=O)c2cccc(N)c2)n1]

A NMP (0.8 mL) mixture of 2-chloro-5-(4-methylpiperazin-1-yl)phenol (approximately 39.29 mg, 0.1733 mmol), N-[4-chloro-6-(2-isopropylphenyl)pyrimidin-2-yl]-3-nitro-benzenesulfonamide (25 mg, 0.05775 mmol), and Cs₂CO₃ (approximately 75.26 mg, 0.2310 mmol) was heated to 110° C. for 16 hours and then cooled to room temperature. The mixture was diluted with water (0.5 mL) and ethyl acetate (1.5 mL) is added and the organic layer was separated, dried with anhydrous sodium sulfate, filtered, and concentrated in vacuo. The resulting residue was taken up in EtOH (0.9 mL) and HCl (0.1 mL of 0.5 M) was added and the mixture was heated to 50° C. for 10 minutes. The solution was filtered and the resulting residues was dissolved in 0.8 mL MeOH, and purified by reverse phase chromatography using a 15 minute gradient of 1% MeCN in water to 99% MeCN to give 3-amino-N-[4-[2-chloro-5-(4-methylpiperazin-1-yl)phenoxy]-6-(2-isopropylphenyl)pyrimidin-2-yl]benzenesulfonamide (dihydrochloride salt) (4.2 mg, 11%). ESI-MS m/z calc. 592.20233, found 593.53 (M+1)⁺; Retention time: 1.49 minutes (LC method A).

### Example 23: Preparation of Compound 74

### Step 1: 3-Amino-N-[4-(2-isopropylphenyl)-6-(3-methylphenoxy)pyrimidin-2-yl]benzenesulfonamide (Compound 74)

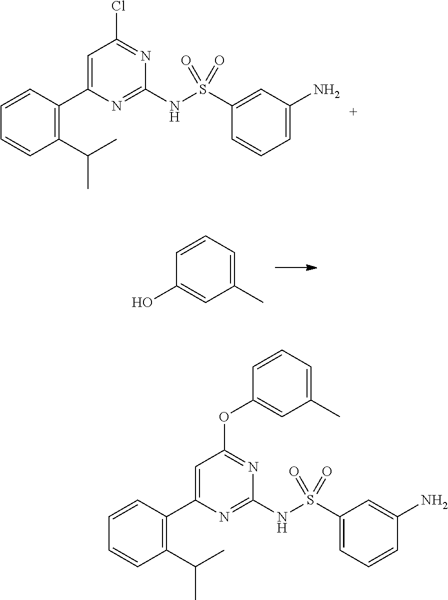

A NMP (0.5 mL) mixture of m-cresol (approximately 12.08 mg, 0.1117 mmol), 3-amino-N-[4-chloro-6-(2-isopropylphenyl)pyrimidin-2-yl]benzenesulfonamide (15 mg, 0.03723 mmol), and Cs₂CO₃ (approximately 48.51 mg, 0.1489 mmol) was stirred at 110° C. for 16 hours and then cooled to room temperature. The solution was filtered, and the resulting residue was dissolved in 0.8 mL MeOH, and purified by reverse phase chromatography using a 15 minute gradient of 1% MeCN in water to 99% MeCN (HCl modifier) to give 3-amino-N-[4-(2-isopropylphenyl)-6-(3-methylphenoxy)pyrimidin-2-yl]benzenesulfonamide (hydrochloride salt). (9.4 mg, 53%). ESI-MS m/z calc. 474.17255, found 475.26 (M+1)⁺; Retention time: 1.87 minutes (LC method A).

### Example 24: Preparation of Compound 75

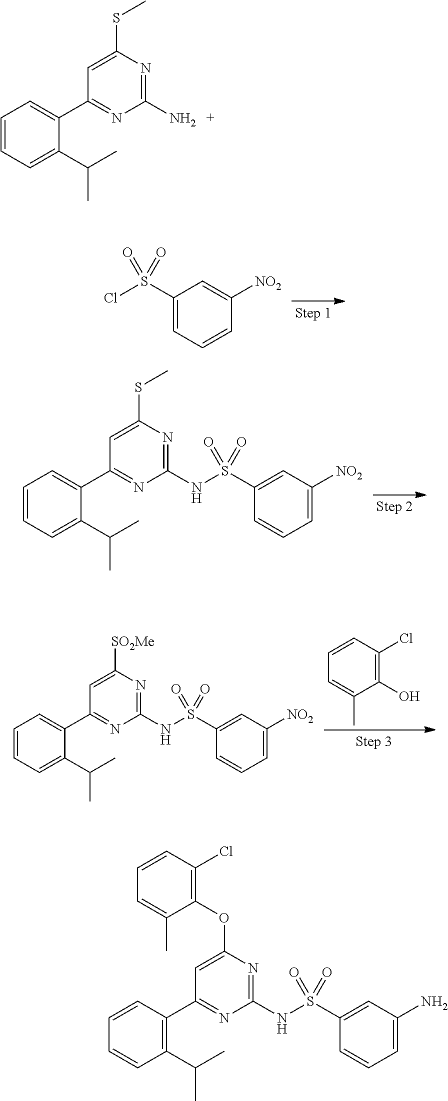

### Step 1: N-[4-(2-Isopropylphenyl)-6-methylsulfanyl-pyrimidin-2-yl]-3-nitro-benzenesulfonamide

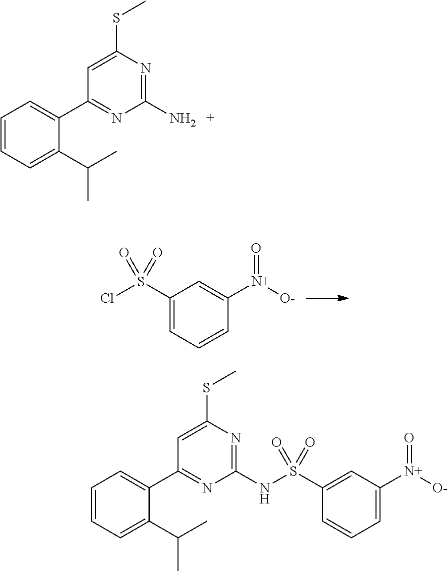

To a heat-gun-dried 20 mL microwave vial equipped with a magnetic stir bar were added 4-(2-isopropylphenyl)-6-methylsulfanyl-pyrimidin-2-amine (501.7 mg, 1.934 mmol) and dimethylformamide (6 mL), and was cooled to 0° C. 60% NaH (approximately 328.8 mg, 8.220 mmol) was added in one portion, and the reaction mixture was warmed to room temperature over 15 minutes. The mixture was cooled to 0° C., upon which 3-nitrobenzenesulfonyl chloride (approximately 655.8 mg, 2.959 mmol) was added in one portion. This solution was stirred at room temperature for 50 minutes, then quenched by a slow transfer onto ice-cold water (ca. 20 mL). The mixture was extracted with ethyl acetate (3×20 mL). The combined organic extracts were washed with 2 N HCl (20 mL), water (20 mL) and saturated aqueous sodium chloride solution (20 mL), then dried over sodium sulfate, filtered, and evaporated in vacuo. This crude product was purified by silica gel chromatography (12 g of silica, 0 to 20% gradient of ethyl acetate/hexanes) to give a dark orange oil that became a foam under high vacuum, N-[4-(2-isopropylphenyl)-6-methylsulfanyl-pyrimidin-2-yl]-3-nitro-benzenesulfonamide (435.8 mg, 51%). ESI-MS m/z calc. 444.0926, found 445.2 (M+1)⁺; Retention time: 0.7 minutes (LC method D).

### Step 2: N-[4-(2-Isopropylphenyl)-6-methylsulfonyl-pyrimidin-2-yl]-3-nitro-benzenesulfonamide

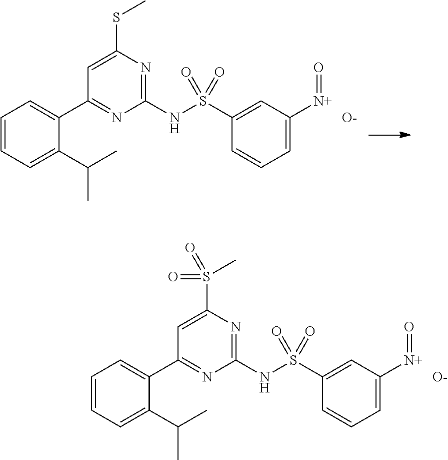

To a 20 mL vial equipped with a magnetic stir bar, N-[4-(2-isopropylphenyl)-6-methylsulfanyl-pyrimidin-2-yl]-3-nitro-benzenesulfonamide (299.5 mg, 0.6737 mmol) and dichloromethane (7.5 mL) were added, followed by m-CPBA (370.5 mg, 1.653 mmol). This solution was stirred at room temperature for 1 hour. The reaction mixture was quenched with solid sodium thiosulfate (850.2 mg, 5.377 mmol). This mixture was stirred for another 1 h at room temperature. The reaction mixture was diluted with dichloromethane (30 mL), then washed with water (30 mL) and saturated aqueous sodium chloride solution (30 mL). The organic layer was then dried over sodium sulfate, filtered, and evaporated in vacuo. This crude product was purified by silica gel chromatography (12 g of silica, 0 to 40% gradient of ethyl acetate/hexanes) to give three batches: 1) 25.4 mg of pure over-oxidized side product; 2) 73.8 mg of a 3:1 mix of product to over-oxidized side product (54.9 mg to 18.9 mg, respectively); and 3) 190.3 mg of pure product. Total of product+side product: N-[4-(2-isopropylphenyl)-6-methylsulfonyl-pyrimidin-2-yl]-3-nitro-benzenesulfonamide (245.2 mg, 76%) ESI-MS m/z calc. 476.08243, found 477.2 (M+1)⁺; Retention time: 0.65 minutes (LC method D), and [6-(2-isopropylphenyl)-2-[(3-nitrophenyl)sulfonylamino]pyrimidin-4-yl] methanesulfonate (44.3 mg, 13%) ESI-MS m/z calc. 492.07733, found 493.2 (M+1)⁺; Retention time: 0.68 minutes (LC method D).

### Step 3: 3-Amino-N-[4-(2-chloro-6-methyl-phenoxy)-6-(2-isopropylphenyl)pyrimidin-2-yl]benzenesulfonamide (Compound 75)

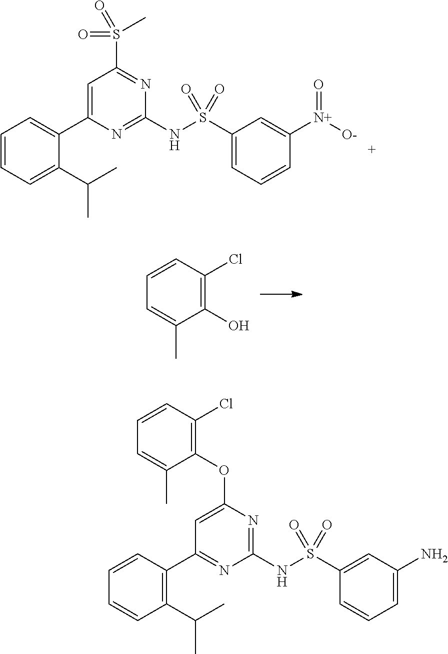

To a 10 mL vial equipped with a magnetic stir bar, N-[4-(2-isopropylphenyl)-6-methylsulfonyl-pyrimidin-2-yl]-3-nitro-benzenesulfonamide (25.0 mg, 0.05246 mmol), N-methylpyrrolidinone (500 μL) and 2-chloro-6-methyl-phenol (28.52 mg, 0.20 mmol) were added, followed by potassium carbonate (30.0 mg, 0.2171 mmol). This mixture was stirred at 110° C. for 19 hours. The reaction mixture was then cooled to room temperature, quenched with 1 N HCl (1 mL), and extracted with ethyl acetate (3×1 mL). The combined organic extracts was washed with water (2×2 mL) and saturated aqueous sodium chloride solution (2 mL), then dried over sodium sulfate, filtered, and evaporated in vacuo. The crude product from the first step was dissolved in ethanol (700 μL) and transferred to a 10 mL vial equipped with a magnetic stir bar. Aqueous HCl (100.0 μL of 0.5 M, 0.05 mmol) was added, followed by a fine dust of iron (20.0 mg, 0.3581 mmol). This reaction mixture was stirred at 60° C. for 10 minutes. It was cooled to room temperature, filtered, and purified by reverse phase HPLC (1-99% acetonitrile in water using HCl as a modifier) to give the desired product 3-Amino-N-[4-(2-chloro-6-methyl-phenoxy)-6-(2-isopropylphenyl)pyrimidin-2-yl]benzenesulfonamide (hydrochloride salt) (12.2mg, 43%). ESI-MS m/z calc. 508.13358, found 509.1 (M+1)⁺; Retention time: 1.98 minutes (LC method A).

### Example 25: Preparation of Compound 76

### Step 1: 2-Chloro-3-(1-methyl-3,6-dihydro-2H-pyridin-4-yl)phenol

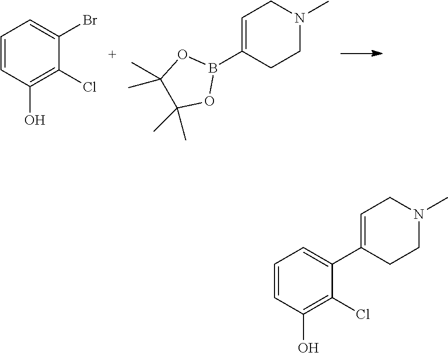

A heterogeneous solution consisting of 3-bromo-2-chloro-phenol (60 mg, 0.2892 mmol), 1-methyl-4-(4,4,5,5-tetramethyl-1,3,2-dioxaborolan-2-yl)-3,6-dihydro-2H-pyridine (50 mg, 0.2241 mmol), potassium carbonate (155 mg, 1.122 mmol) and Pd(dppf)Cl₂(36 mg, 0.04408 mmol) in dioxane (1 mL) and water (200 μL) was microwaved in a sealed vial to 120° C. for 10 minutes. The reaction mixture was acidified with acetic acid (202 mg, 3.364 mmol), further diluted with DMSO (1.0 mL) and filtered. The crude solution was purified by HPLC (acetonitrile in water with 0.1% hydrochloric acid) to afford 2-chloro-3-(1-methyl-3,6-dihydro-2H-pyridin-4-yl)phenol (23.2 mg, 46%) as a white solid. ESI-MS m/z calc. 223.07639, found 224.21 (M+1)⁺; Retention time: 0.31 minutes (LC method D).

### Step 2: 3-Amino-N-[4-[2-chloro-3-(1-methyl-4-piperidyl)phenoxy]-6-(2-isopropylphenyl)pyrimidin-2-yl]benzenesulfonamide (Compound 76)

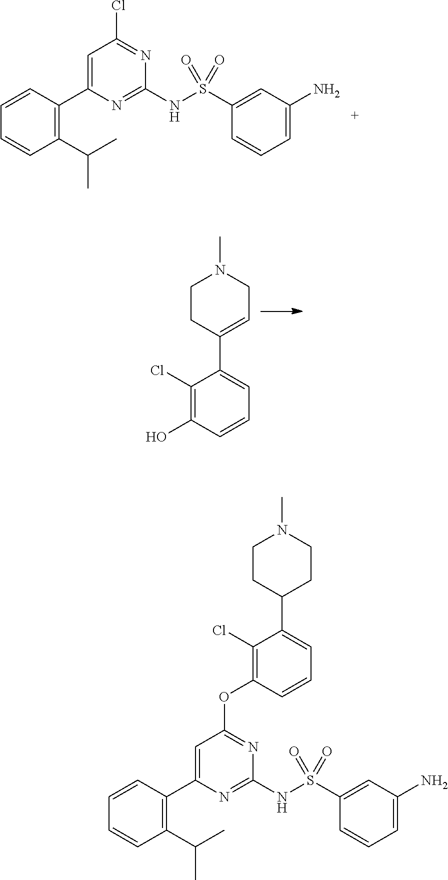

Stage 1: To a solution of 2-chloro-3-(1-methyl-3,6-dihydro-2H-pyridin-4-yl)phenol (23.2 mg, 0.1037 mmol) in methanol (1.0 mL) was added platinum (40.5 mg of 5% w/w, 0.01038 mmol)(sulfided). The reaction flask was fitted with a balloon filled with hydrogen and stirred for 16 hours. The reaction mixture was filtered and concentrated in vacuo to afford 2-chloro-3-(1-methyl-4-piperidyl)phenol and was used without further purification. ESI-MS m z calc. 225.09204, found 226.24 (M+1)⁺; Retention time: 0.32 minutes (LC method D).

Stage 2: A heterogeneous solution consisting of 2-chloro-3-(1-methyl-4-piperidyl)phenol, 3-amino-N-[4-chloro-6-(2-isopropylphenyl)pyrimidin-2-yl]benzenesulfonamide (20.9 mg, 0.05187 mmol), and potassium carbonate (35.8 mg, 0.2590 mmol) in NMP (0.5 mL) was heated to 110° C. for 16 hours. The reaction mixture was cooled and acidified using acetic acid (90 μL, 1.583 mmol), diluted with water (200 μL) and DMSO (0.3 mL) and filtered through a 0.45 μM PTFE syringe filter. The crude solution was separated by HPLC (gradient: 1 to 99% acetonitrile in water with 0.1% hydrochloric acid) to afford 3-amino-N-[4-[2-chloro-3-(1-methyl-4-piperidyl)phenoxy]-6-(2-isopropylphenyl)pyrimidin-2-yl]benzenesulfonamide (hydrochloride salt) (6.8 mg, 10%) as a white solid. ESI-MS m/z calc. 591.2071, found 592.59 (M+1)⁺; Retention time: 1.58 minutes (LC method A).

### Example 26: Preparation of Compound 77

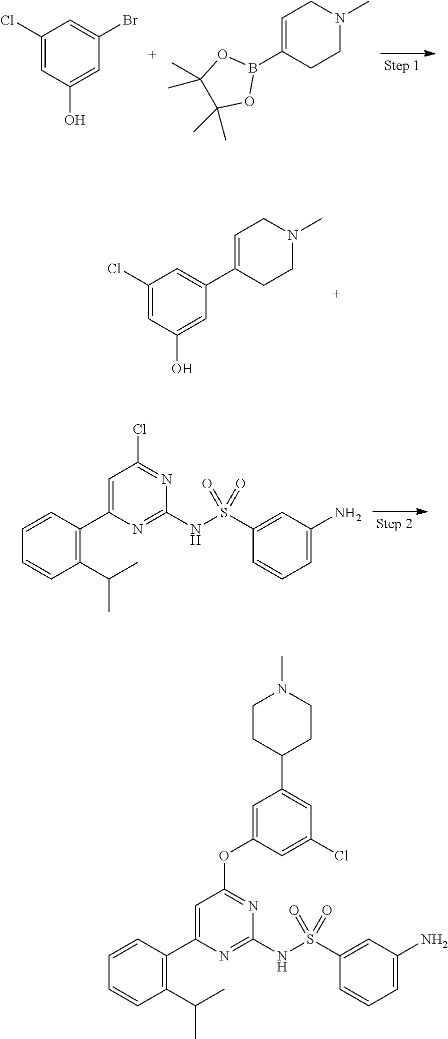

### Step 1: 3-Chloro-5-(1-methyl-3,6-dihydro-2H-pyridin-4-yl)phenol

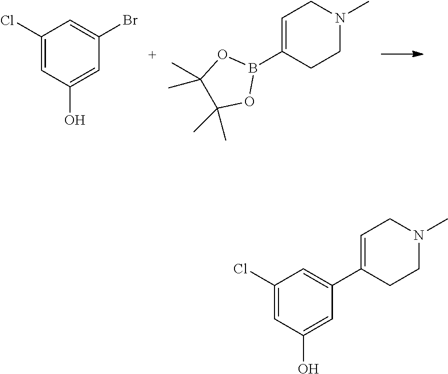

A heterogeneous solution consisting of 3-bromo-5-chloro-phenol (62 mg, 0.2989 mmol), 1-methyl-4-(4,4,5,5-tetramethyl-1,3,2-dioxaborolan-2-yl)-3,6-dihydro-2H-pyridine (50 mg, 0.2241 mmol), potassium carbonate (155 mg, 1.122 mmol) and Pd(dppf)Cl₂ (37 mg, 0.04531 mmol) in dioxane (1 mL) and water (0.2 mL) was microwaved in a sealed vial to 120° C. for 10 minutes. The reaction mixture was acidified with acetic acid (202 mg, 3.364 mmol), further diluted with DMSO (1.0 mL) and filtered. The crude solution was separated by HPLC (acetonitrile in water with 0.1% hydrochloric acid) to afford 3-chloro-5-(1-methyl-3,6-dihydro-2H-pyridin-4-yl)phenol (21 mg, 42%) as a white solid. ESI-MS m/z calc. 223.07639, found 224.21 (M+1)⁺; Retention time: 0.35 minutes (LC method D).

### Step 2: 3-Amino-N-[4-[3-chloro-5-(1-methyl-4-piperidyl)phenoxy]-6-(2-isopropylphenyl)pyrimidin-2-yl]benzenesulfonamide (Compound 77)

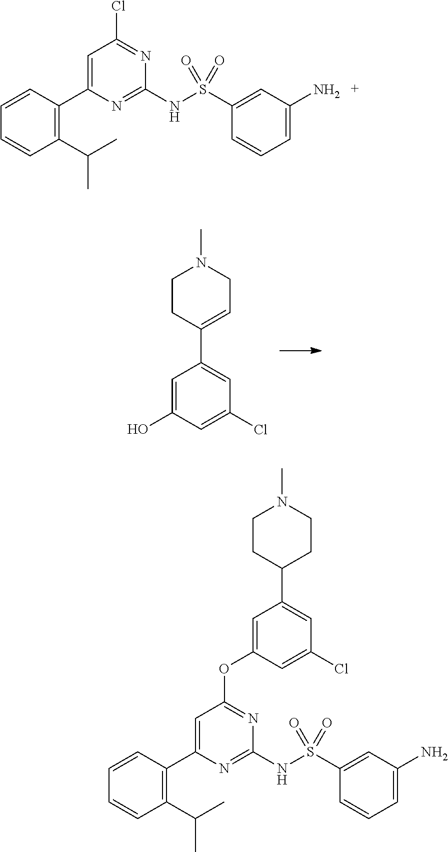

To a solution of 3-chloro-5-(1-methyl-3,6-dihydro-2H-pyridin-4-yl)phenol (21 mg, 0.09388 mmol) in methanol (940 μL) was added platinum (36.6 mg of 5% w/w, 0.009381 mmol)(sulfided). The reaction flask was fitted with a balloon filled with hydrogen and stirred for 16 hours. The reaction mixture was filtered and concentrated in vacuo to afford 3-chloro-5-(1 -methyl-4-piperidyl)phenol and was used without further purification. ESI-MS m/z calc. 225.09204, found 226.24 (M+1)⁺; Retention time: 0.36 minutes (LC method D).

A heterogeneous solution consisting of 3-chloro-5-(1-methyl-4-piperidyl)phenol, 3-amino-N-[4-chloro-6-(2-isopropylphenyl)pyrimidin-2-yl]benzenesulfonamide (18.9 mg, 0.04691 mmol), and potassium carbonate (32.4 mg, 0.2344 mmol) in NMP (0.5 mL) was heated to 110° C. for 16 hours. The reaction mixture was cooled and acidified using acetic acid (50.0 μL, 0.8792 mmol), diluted with water (200 μL) and DMSO (0.3 mL) and filtered through a 0.45 μM PTFE syringe filter. The crude solution was separated by HPLC (gradient: 1 to 99% acetonitrile in water with 0.1% hydrochloric acid) to afford 3-amino-N-[4-[3-chloro-5-(1-methyl-4-piperidyl)phenoxy]-6-(2-isopropylphenyl)pyrimidin-2-yl]benzenesulfonamide (hydrochloride salt) (4.6 mg, 8%) as a white solid. ESI-MS m/z calc. 591.2071, found 592.55 (M+1)⁺; Retention time: 1.61 minutes (LC method A).

### Example 27: Preparation of Compound 78

### Step 1: 2-Chloro-4-(1-methyl-3,6-dihydro-2H-pyridin-4-yl)phenol

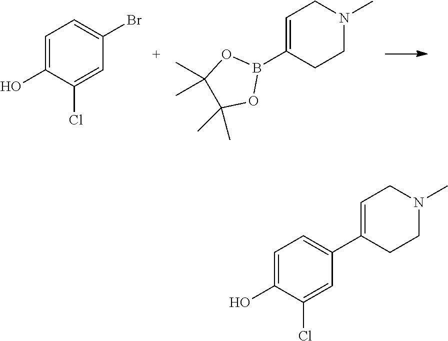

A heterogeneous solution consisting of 4-bromo-2-chloro-phenol (60 mg, 0.2892 mmol), 1-methyl-4-(4,4,5,5-tetramethyl-1,3,2-dioxaborolan-2-yl)-3,6-dihydro-2H-pyridine (50mg, 0.2241 mmol), potassium carbonate (155 mg, 1.122 mmol) and Pd(dppf)Cl₂(36 mg, 0.04408 mmol) in dioxane (1 mL) and water (200 μL) was microwaved in a sealed vial to 120° C. for 10 minutes. The reaction mixture was acidified with acetic acid (202 mg, 3.364 mmol), further diluted with DMSO (1.0 mL) and filtered. The crude solution was separated by HPLC (acetonitrile in water with 0.1% hydrochloric acid) to afford 2-chloro-4-(1-methyl-3,6-dihydro-2H-pyridin-4-yl)phenol (10.7 mg, 15%) as a white solid. ESI-MS m/z calc. 223.07639, found 224.21 (M+1)⁺; Retention time: 0.31 minutes (LC method D).

### Step 2: 3-Amino-N-[4-[2-chloro-4-(1-methyl-4-piperidyl)phenoxy]-6-(2-isopropylphenyl)pyrimidin-2-yl]benzenesulfonamide (Compound 78)

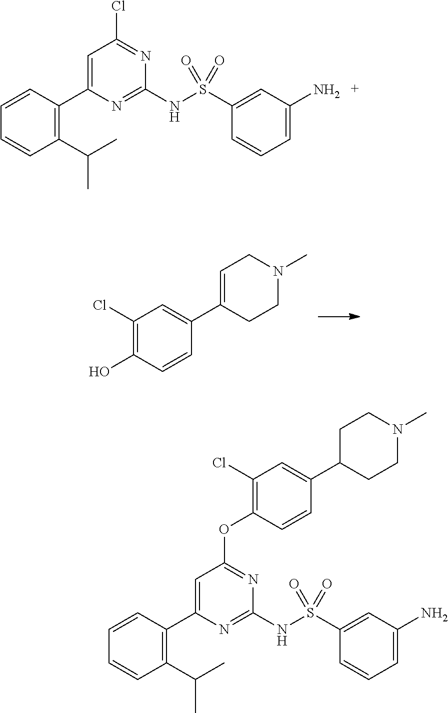

To a solution of 2-chloro-4-(1-methyl-3,6-dihydro-2H-pyridin-4-yl)phenol (10.7 mg, 0.04783 mmol) in methanol (480 μL) was added platinum (18.7 mg of 5% w/w, 0.004793 mmol)(sulfided). The reaction flask was fitted with a balloon filled with hydrogen and stirred for 16 hours. The reaction mixture was filtered and concentrated in vacuo to afford 2-chloro-4-(1 -methyl-4-piperidyl)phenol and was used without further purification. ESI-MS m/z calc. 225.09204, found 226.24 (M+1)⁺; Retention time: 0.32 minutes (LC method D).

A heterogeneous solution consisting of 2-chloro-4-(1-methyl-4-piperidyl)phenol, 3-amino-N-[4-chloro-6-(2-isopropylphenyl)pyrimidin-2-yl]benzenesulfonamide (19.3 mg, 0.04790 mmol) and potassium carbonate (33.1 mg, 0.2395 mmol) in NMP (0.5 mL) was heated to 110° C. for 16 hours. The reaction mixture was cooled and acidified using acetic acid (40 μL, 0.7034 mmol), diluted with water (200 μL) and DMSO (0.3 mL) and filtered through a 0.45 μM PTFE syringe filter. The crude solution was separated by HPLC (gradient: 1 to 99% acetonitrile in water with 0.1% hydrochloric acid) to afford 3-amino-N-[4-[2-chloro-4-(1-methyl-4-piperidyl)phenoxy]-6-(2-isopropylphenyl)pyrimidin-2-yl]benzenesulfonamide (hydrochloride salt) (2.2 mg, 7%) as a white solid. ESI-MS m/z calc. 591.2071, found 592.55 (M+1)⁺; Retention time: 1.61 minutes (LC method A).

### Example 28: Preparation of Compound 79

### Step 1: 2-Chloro-5-(1-methyl-3,6-dihydro-2H-pyridin-4-yl)phenol

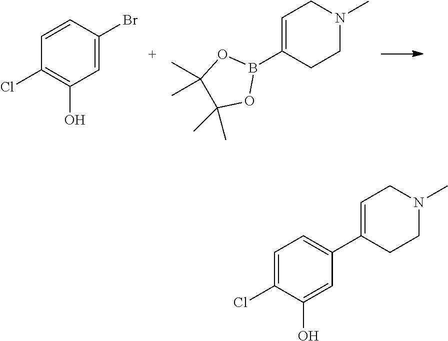

A heterogeneous solution consisting of 5-bromo-2-chloro-phenol (60 mg, 0.2892 mmol), 1-methyl-4-(4,4,5,5-tetramethyl-1,3,2-dioxaborolan-2-yl)-3,6-dihydro-2H-pyridine (50 mg, 0.2241 mmol), potassium carbonate (155 mg, 1.122 mmol) and Pd(dppf)Cl₂ (36 mg, 0.04408 mmol) in dioxane (1 mL) and water (200 μL) was microwaved in a sealed vial to 120° C. for 10 minutes. The reaction mixture was acidified with acetic acid (190 μL, 3.341 mmol), further diluted with DMSO (1.0 mL) and filtered. The crude solution was separated by HPLC (gradient: 1 to 99% acetonitrile in water with 0.1% hydrochloric acid) to afford 2-chloro-5-(1 -methyl-3,6-dihydro-2H-pyridin-4-yl)phenol (27 mg, 45%) as a white solid. ESI-MS m/z calc. 223.07639, found 224.21 (M+1)⁺; Retention time: 0.31 minutes. (LC method D).

### Step 2: 3-Amino-N-[4-[2-chloro-5-(1-methyl-4-piperidyl)phenoxy]-6-(2-isopropylphenyl)pyrimidin-2-yl]benzenesulfonamide (Compound 79)

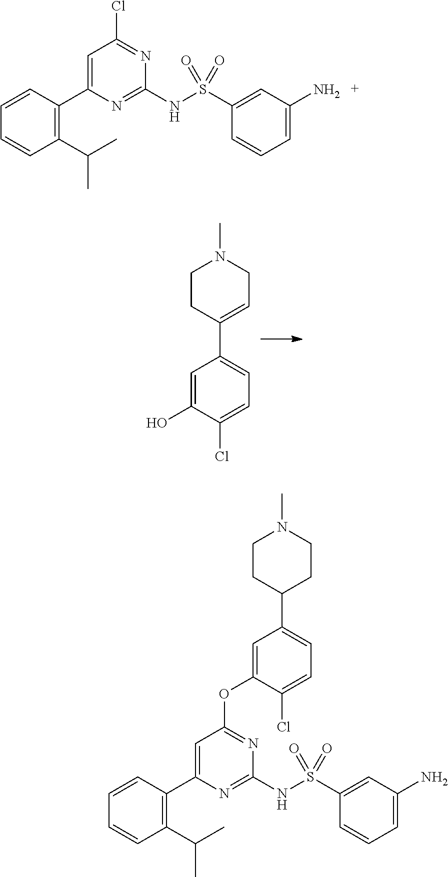

To a solution of 2-chloro-5-(1-methyl-3,6-dihydro-2H-pyridin-4-yl)phenol (27 mg, 0.1207 mmol) in methanol (1.2 mL) was added platinum (47.1 mg of 5% w/w, 0.01207 mmol)(sulfided). The reaction flask was fitted with a balloon filled with hydrogen and stirred for 16 hours. The reaction mixture was filtered and concentrated in vacuo to afford 2-chloro-5-(1 -methyl-4-piperidyl)phenol and was used without further purification. ESI-MS m/z calc. 225.09204, found 226.24 (M+1)⁺; Retention time: 0.34 minutes (LC method D).

A heterogeneous solution consisting of 2-chloro-5-(1-methyl-4-piperidyl)phenol, 3-amino-N-[4-chloro-6-(2-isopropylphenyl)pyrimidin-2-yl]benzenesulfonamide (19.5 mg, 0.04840 mmol), and potassium carbonate (40.0 mg, 0.2894 mmol) in NMP (0.6 mL) was heated to 110°C. for 16 hours. The reaction mixture was cooled and acidified using acetic acid (100 μL, 1.758 mmol), diluted with water (200 μL) and DMSO (0.3 mL) and filtered through a 0.45 μM PTFE syringe filter. The crude solution was separated by HPLC (gradient: 1 to 99% acetonitrile in water with 0.1% hydrochloric acid) to afford 3-amino-N-[4-[2-chloro-5-(1-methyl-4-piperidyl)phenoxy]-6-(2-isopropylphenyl)pyrimidin-2-yl]benzenesulfonamide (hydrochloride salt) (2.9 mg, 4%) as a white solid. ESI-MS m/z calc. 591.2071, found 592.55 (M+1)⁺; Retention time: 1.57 minutes (LC method A).

### Example 29: Preparation of Compound 80

### Step 1: 3-Amino-N-[4-[2-chloro-4-(4-methylpiperazin-1-yl)phenoxy]-6-(2-isopropylphenyl)pyrimidin-2-yl]benzenesulfonamide (Compound 80)

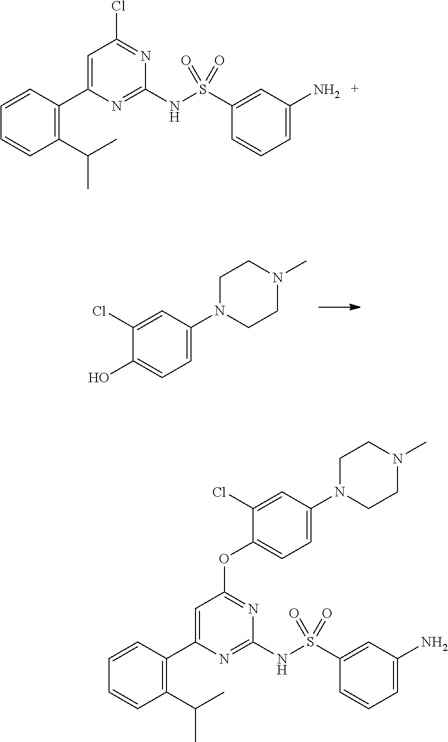

An NMP (0.4 mL) mixture of 2-chloro-4-(4-methylpiperazin-1-yl)phenol (24.8 mg, 0.1094 mmol), 3-amino-N-[4-chloro-6-(2-isopropylphenyl)pyrimidin-2-yl]benzenesulfonamide (15.1 mg, 0.03748 mmol), and Cs₂CO₃ (71.2 mg, 0.2185 mmol) was stirred at 110° C. for 16 hours and then cooled to room temperature. The solution was filtered and the filtrate dissolved in 0.8 mL MeOH, and purified by reverse phase chromatography using a 15 minute gradient of 20% MeCN in water to 80% MeCN (HCl modifier) to give 3-amino-N-[4-[2-chloro-4-(4-methylpiperazin-1-yl)phenoxy]-6-(2-isopropylphenyl)pyrimidin-2-yl]benzenesulfonamide (dihydrochloride salt) (5.8 mg, 23%). ¹H NMR (400 MHz, DMSO-d₆) δ 10.79 (s, 1H), 7.48-7.41 (m, 2H), 7.33-7.08 (m, 7H), 7.02 (s, 1H), 6.78 (d, J=7.8 Hz, 1H), 6.61 (s, 1H), 3.93 (d, J=9.2 Hz, 2H), 3.50 (d, J=8.4 Hz, 2H), 3.24-3.07 (m, 4H), 2.82 (d, J=3.2 Hz, 3H), 1.10 (d, J=6.8 Hz, 6H). ESI-MS m/z calc. 592.20233, found 593.3 (M+1)⁺; Retention time: 1.4 minutes (LC method A).

### Example 30: Preparation of Compound 81

### Step 1: 2,5-Dichloro-4-(4-methylpiperazin-1-yl)phenol

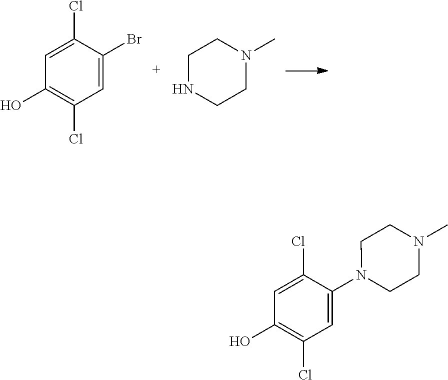

A dioxane (1 mL) mixture of 4-bromo-2,5-dichloro-phenol (50 mg, 0.2067 mmol), 1-methylpiperazine (approximately 22.78 mg, 0.2274 mmol), chloro(2-di-t-butylphosphino-2′, 4′, 6′-tri-i-propyl-1,1′-biphenyl)[2-(2-aminoethyl)phenyl] palladium(II) (t-BuXPhos Palladacycle Gen. 1) (approximately 13.46 mg, 0.02067 mmol), and sodium tert-butoxide (approximately 49.67 mg, 0.5168 mmol) was sparged with nitrogen and then stirred at 35° C. for 2 hours. The solution was filtered and the filtrate was dissolved in 0.8 mL MeOH, and purified by reverse phase chromatography using a 15 minute gradient of 1% MeCN in water to 50% MeCN (HCl modifier) to give 2,5-dichloro-4-(4-methylpiperazin-1-yl)phenol (hydrochloride salt) (20.4 mg, 33%).

### Step 2: 3-Amino-N-[4-[2,5-dichloro-4-(4-methylpiperazin-1-yl)phenoxy]-6-(2-isopropylphenyl)pyrimidin-2-yl]benzenesulfonamide (Compound 81)

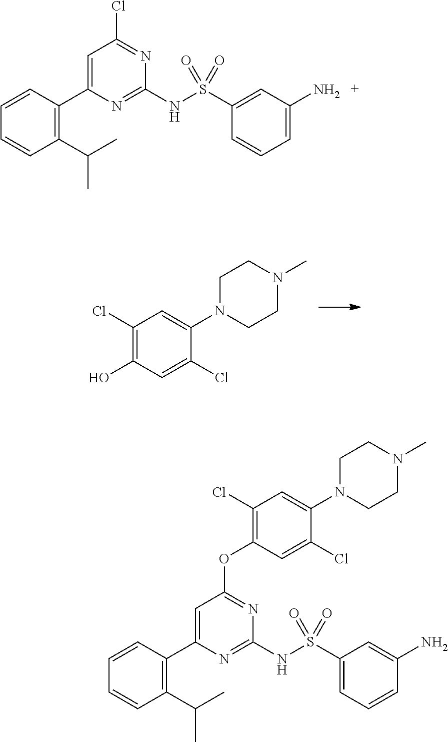

A NMP (0.5 mL) mixture of 2,5-dichloro-4-(4-methylpiperazin-1-yl)phenol (hydrochloride salt) (20.4 mg, 0.0685 mmol), 3-amino-N-[4-chloro-6-(2-isopropylphenyl)pyrimidin-2-yl]benzenesulfonamide (12.6 mg, 0.0313 mmol), and Cs₂CO₃ (approximately 61.3 mg, 0.188 mmol) was stirred at 110° C. for 16 hours and then cooled to room temperature. The solution was filtered and the resulting residue was dissolved in 0.8 mL MeOH, and purified by reverse phase chromatography using a 15 minute gradient of 1% MeCN in water to 99% MeCN (HCl modifier) to give 3-amino-N-[4-[2,5-dichloro-4-(4-methylpiperazin-1-yl)phenoxy]-6-(2-isopropylphenyl)pyrimidin-2-yl]benzenesulfonamide (dihydrochloride salt) (4.0 mg, 18%). ESI-MS m/z calc. 626.1634, found 627.53 (M+1)⁺; Retention time: 1.59 minutes (LC method A).

### Example 31: Preparation of Compound 82

### Step 1: 4-(1-Methyl-4-piperidyl)-3-(trifluoromethyl)phenol

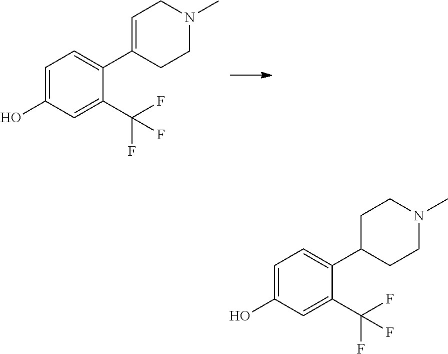 [SMILES: CN1CC=C(c2ccc(O)cc2C(F)(F)F)CC1 ; CN1CCC(c2ccc(O)cc2C(F)(F)F)CC1]

A solution of 4-(1-methyl-3,6-dihydro-2H-pyridin-4-yl)-3-(trifluoromethyl)phenol (56 mg, 0.2177 mmol) and platinum (approximately 169.9 mg of 5% w/w, 0.04354 mmol) in methanol (2.177 mL) was stirred under an atmosphere of hydrogen gas for 12 hour. The reaction was filtered, and the volatiles removed in vacuo. The crude intermediate 4-(1-methyl-4-piperidyl)-3-(trifluoromethyl)phenol was used without further purification.

### Step 2: 3-Amino-N-[4-(2-isopropylphenyl)-6-[4-(1-methyl-4-piperidyl)-3-(trifluoromethyl)phenoxy]pyrimidin-2-yl]benzenesulfonamide (Compound 82)

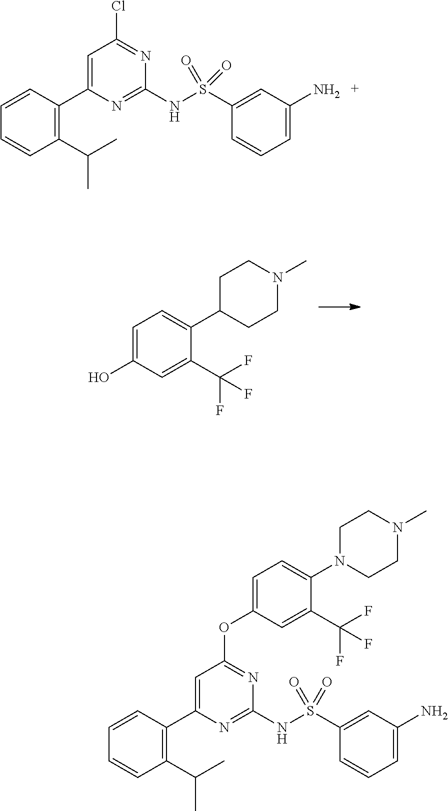

An NMP (0.6 mL) mixture of 3-amino-N-[4-chloro-6-(2-isopropylphenyl)pyrimidin-2-yl]benzenesulfonamide (16.2 mg, 0.04021 mmol), 4-(1-methyl-4-piperidyl)-3-(trifluoromethyl)phenol (approximately 53.77 mg, 0.2074 mmol), and Cs₂CO₃ (approximately 66.30 mg, 0.2035 mmol) was mixed at 110° C. for 16 hours and then cooled to room temperature. The solution was filtered and the filtrate dissolved in 0.8 mL MeOH, and purified by reverse phase chromatography using a 15 minute gradient of 20% MeCN in water to 80% MeCN (HCl modifier) to give 3-amino-N-[4-(2-isopropylphenyl)-6-[4-(1-methyl-4-piperidyl)-3-(trifluoromethyl)phenoxy]pyrimidin-2-yl]benzenesulfonamide (dihydrochloride salt) (7.4 mg, 5%). ESI-MS m/z calc. 625.23346, found 626.76 (M+1)⁺; Retention time: 1.64 minutes (LC method A).

### Example 32: Preparation of Compound 83

### Step 1: 4-Chloro-3-(1-methyl-3,6-dihydro-2H-pyridin-4-yl)phenol

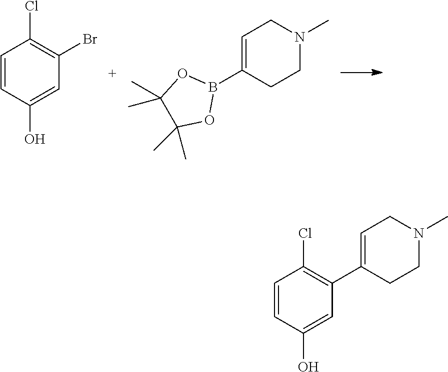

A heterogeneous solution consisting of 3-bromo-4-chloro-phenol (60 mg, 0.2892 mmol), 1-methyl-4-(4,4,5,5-tetramethyl-1,3,2-dioxaborolan-2-yl)-3,6-dihydro-2H-pyridine (50 mg, 0.2241 mmol), potassium carbonate (155 mg, 1.122 mmol) and Pd(dppf)Cl₂ (36 mg, 0.04408 mmol) in dioxane (1 mL) and water (200 μL) was microwaved in a sealed vial to 120° C. for 10 minutes. The reaction mixture was acidified with acetic acid (202 mg, 3.364 mmol), further diluted with DMSO (1.0 mL) and filtered. The crude solution was separated by HPLC (acetonitrile in water with 0.1% hydrochloric acid) to afford 4-chloro-3-(1-methyl-3,6-dihydro-2H-pyridin-4-yl)phenol (20.5 mg, 13%) as a white solid. ESI-MS m/z calc. 223.07639, found 224.21 (M+1)⁺; Retention time: 0.34 minutes (LC method D).

### Step 2: 3-Amino-N-[4-[4-chloro-3-(1-methyl-4-piperidyl)phenoxy]-6-(2-isopropylphenyl)pyrimidin-2-yl]benzenesulfonamide (Compound 83)

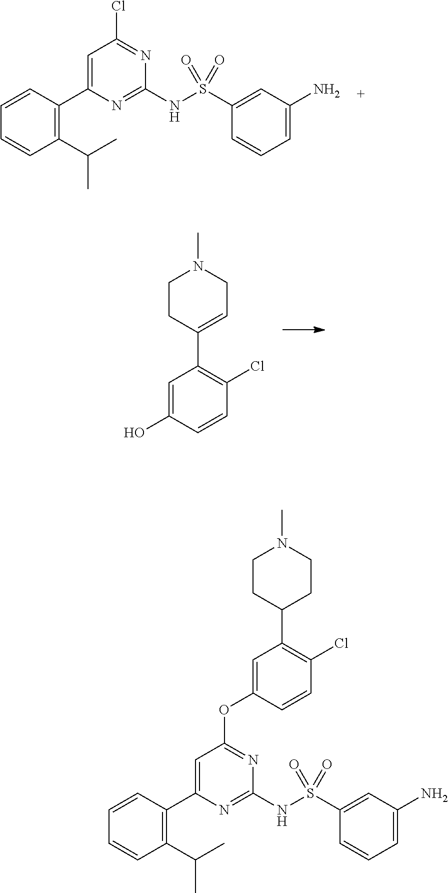 [SMILES: CC(C)c1ccccc1-c1cc(Cl)nc(NS(=O)(=O)c2cccc(N)c2)n1 ; CN1CC=C(c2cc(O)ccc2Cl)CC1 ; CC(C)c1ccccc1-c1cc(Oc2ccc(Cl)c(C3CCN(C)CC3)c2)nc(NS(=O)(=O)c2cccc(N)c2)n1]

To a solution of 4-chloro-3-(1-methyl-3,6-dihydro-2H-pyridin-4-yl)phenol (20.5 mg, 0.09164 mmol) in methanol (920 μL) was added platinum (35.8 mg of 5% w/w, 0.009176 mmol) (sulfided 5 wt %). The reaction flask was fitted with a balloon filled with hydrogen and stirred for 2 hours before filtering through a 0.45 μM PTFE syringe filter. The mother liquor was concentrated in vacuo to afford 4-chloro-3-(1-methyl-4-piperidyl)phenol and was used without further purification. ESI-MS m/z calc. 225.09204, found 226.24 (M+1)⁺; Retention time: 0.36 minutes (LC method D).

To a solution of 4-chloro-3-(1-methyl-4-piperidyl)phenol in NMP (0.5 mL) was added potassium carbonate (31.7 mg, 0.2294 mmol). The reaction mixture was heated to 110° C. in a sealed vial for 16 hour. The solution was acidified using acetic acid (80 μL, 1.407 mmol), diluted with water (200 μL) and DMSO (0.3 mL) and filtered through a 0.45 μM PTFE syringe filter. The crude solution was separated by HPLC (acetonitrile in water with 0.1% hydrochloric acid) to afford 3-amino-N-[4-[4-chloro-3-(1-methyl-4-piperidyl)phenoxy]-6-(2-isopropylphenyl)pyrimidin-2-yl]benzenesulfonamide (hydrochloride salt) (1.2 mg, 20%) as a white solid. ESI-MS m/z calc. 591.2071, found 592.92 (M+1)⁺; Retention time: 2.06 minutes (LC method A).

### Example 33: Characterization of Compounds 84-120

The compounds in the following tables were prepared in a manner analogous to that described above using commercially available reagents and intermediates described herein.

| | | LCMS | | | |
|---|---|---|---|---|---|
| | | Room | | | |
| Compound | | Temperature | Calc. | | LCMS |
| number | Structure | (min) | mass | M + 1 | Method |
| 84 | 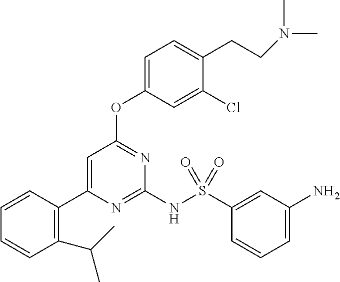 [SMILES: CC(C)c1ccccc1-c1cc(Oc2ccc(CCN(C)C)c(Cl)c2)nc(NS(=O)(=O)c2cccc(N)c2)n1] | 1.38 | 565.191 | 566.2 | A |
| 85 | 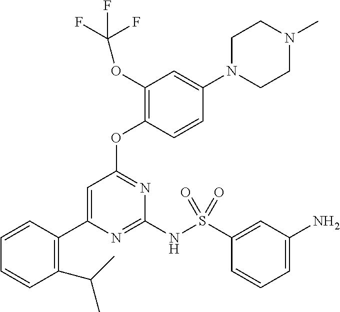 [SMILES: CC(C)c1ccccc1-c1cc(Oc2ccc(N3CCN(C)CC3)cc2OC(F)(F)F)nc(NS(=O)(=O)c2cccc(N)c2)n1] | 1.66 | 642.224 | 643.64 | A |
| 86 | 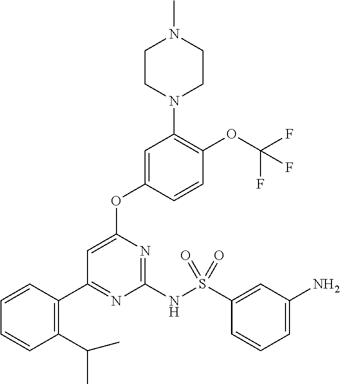 [SMILES: CC(C)c1ccccc1-c1cc(Oc2ccc(OC(F)(F)F)c(N3CCN(C)CC3)c2)nc(NS(=O)(=O)c2cccc(N)c2)n1] | 1.52 | 642.224 | 643.3 | A |
| 87 | 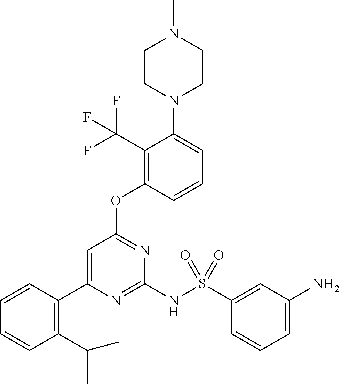 [SMILES: CC(C)c1ccccc1-c1cc(Oc2cccc(N3CCN(C)CC3)c2C(F)(F)F)nc(NS(=O)(=O)c2cccc(N)c2)n1] | 1.57 | 626.229 | 627.67 | A |
| 88 | 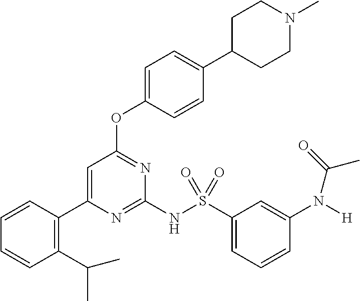 [SMILES: CC(=O)Nc1cccc(S(=O)(=O)Nc2nc(Oc3ccc(C4CCN(C)CC4)cc3)cc(-c3ccccc3C(C)C)n2)c1] | 1.46 | 599.257 | 600.57 | A |
| 89 | 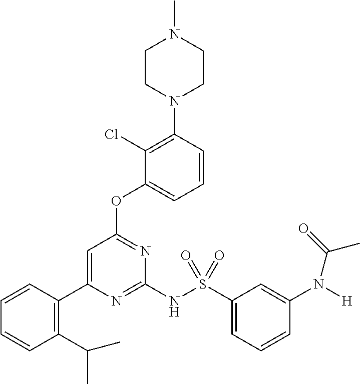 [SMILES: CC(=O)Nc1cccc(S(=O)(=O)Nc2nc(Oc3cccc(N4CCN(C)CC4)c3Cl)cc(-c3ccccc3C(C)C)n2)c1] | 1.5 | 634.213 | 635.52 | A |
| 90 | 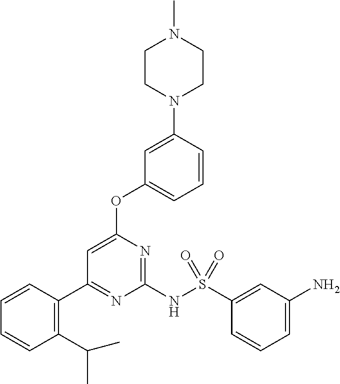 [SMILES: CC(C)c1ccccc1-c1cc(Oc2cccc(N3CCN(C)CC3)c2)nc(NS(=O)(=O)c2cccc(N)c2)n1] | 1.42 | 558.241 | 559.57 | A |
| 91 | 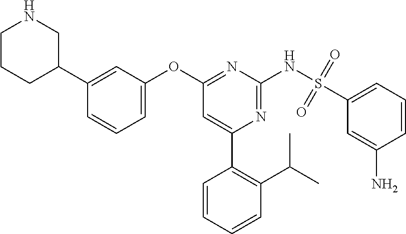 [SMILES: CC(C)c1ccccc1-c1cc(Oc2cccc(C3CCCNC3)c2)nc(NS(=O)(=O)c2cccc(N)c2)n1] | 1.44 | 543.23 | 544.58 | A |
| 92 | 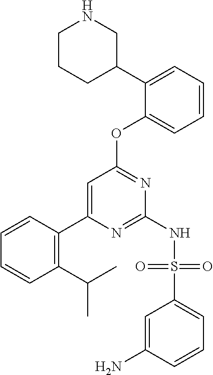 [SMILES: CC(C)c1ccccc1-c1cc(Oc2ccccc2C2CCCNC2)nc(NS(=O)(=O)c2cccc(N)c2)n1] | 1.39 | 543.23 | 544.58 | A |
| 93 | 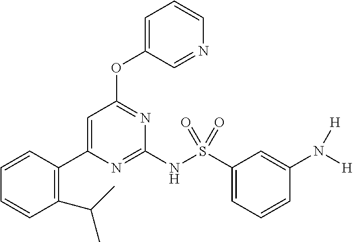 [SMILES: CC(C)c1ccccc1-c1cc(Oc2cccnc2)nc(NS(=O)(=O)c2cccc(N)c2)n1] | 1.46 | 461.152 | 462.44 | A |
| 94 | 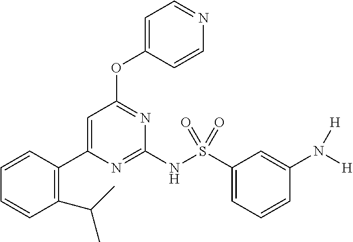 [SMILES: CC(C)c1ccccc1-c1cc(Oc2ccncc2)nc(NS(=O)(=O)c2cccc(N)c2)n1] | 1.42 | 461.152 | 462.44 | A |
| 95 | 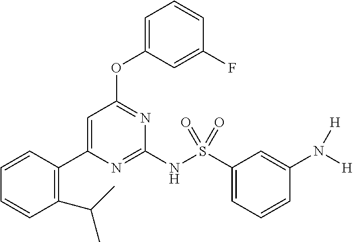 [SMILES: CC(C)c1ccccc1-c1cc(Oc2cccc(F)c2)nc(NS(=O)(=O)c2cccc(N)c2)n1] | 1.83 | 478.147 | 479.23 | A |
| 96 | 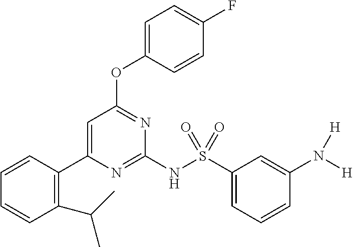 [SMILES: CC(C)c1ccccc1-c1cc(Oc2ccc(F)cc2)nc(NS(=O)(=O)c2cccc(N)c2)n1] | 1.82 | 478.147 | 479.23 | A |
| 97 | 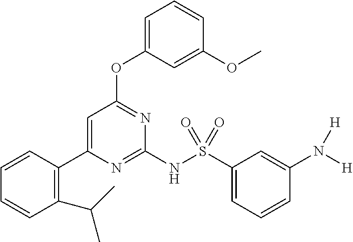 [SMILES: COc1cccc(Oc2cc(-c3ccccc3C(C)C)nc(NS(=O)(=O)c3cccc(N)c3)n2)c1] | 1.78 | 490.167 | 491.27 | A |
| 98 | 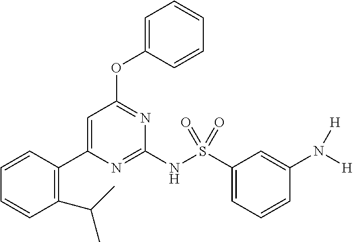 [SMILES: CC(C)c1ccccc1-c1cc(Oc2ccccc2)nc(NS(=O)(=O)c2cccc(N)c2)n1] | 1.77 | 460.157 | 461.25 | A |
| 99 | 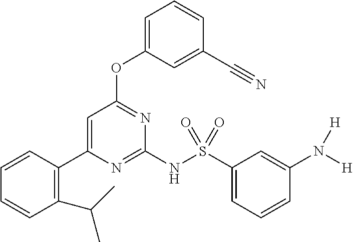 [SMILES: CC(C)c1ccccc1-c1cc(Oc2cccc(C#N)c2)nc(NS(=O)(=O)c2cccc(N)c2)n1] | 1.78 | 485.152 | 486.23 | A |
| 100 | 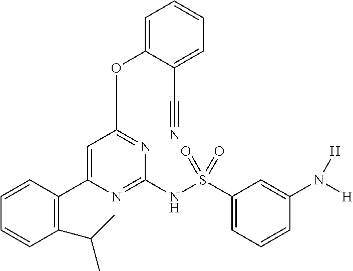 [SMILES: CC(C)c1ccccc1-c1cc(Oc2ccccc2C#N)nc(NS(=O)(=O)c2cccc(N)c2)n1] | 1.83 | 485.152 | 486.5 | A |
| 101 | 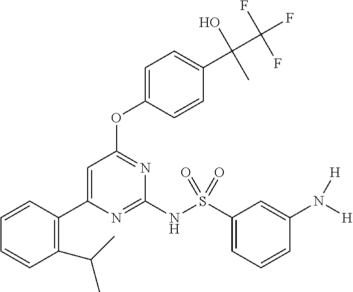 [SMILES: CC(C)c1ccccc1-c1cc(Oc2ccc(C(C)(O)C(F)(F)F)cc2)nc(NS(=O)(=O)c2cccc(N)c2)n1] | 1.97 | 572.171 | 573.51 | A |
| 102 | 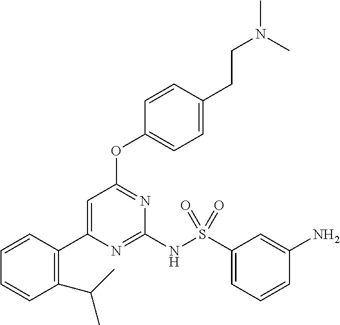 [SMILES: CC(C)c1ccccc1-c1cc(Oc2ccc(CCN(C)C)cc2)nc(NS(=O)(=O)c2cccc(N)c2)n1] | 1.47 | 531.23 | 532.57 | A |
| 103 | 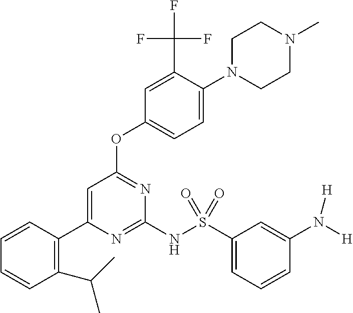 [SMILES: CC(C)c1ccccc1-c1cc(Oc2ccc(N3CCN(C)CC3)c(C(F)(F)F)c2)nc(NS(=O)(=O)c2cccc(N)c2)n1] | 1.6 | 626.229 | 627.64 | A |
| 104 | 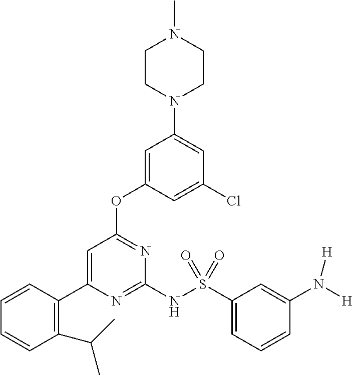 [SMILES: CC(C)c1ccccc1-c1cc(Oc2cc(Cl)cc(N3CCN(C)CC3)c2)nc(NS(=O)(=O)c2cccc(N)c2)n1] | 1.55 | 592.202 | 593.53 | A |
| 105 | 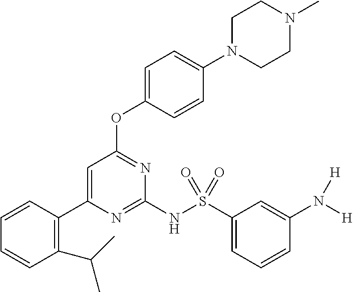 [SMILES: CC(C)c1ccccc1-c1cc(Oc2ccc(N3CCN(C)CC3)cc2)nc(NS(=O)(=O)c2cccc(N)c2)n1] | 1.38 | 558.241 | 559.57 | A |
| 106 | 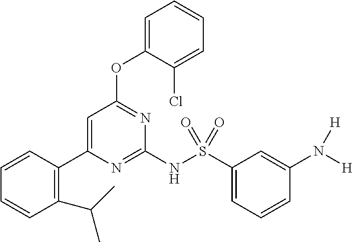 [SMILES: CC(C)c1ccccc1-c1cc(Oc2ccccc2Cl)nc(NS(=O)(=O)c2cccc(N)c2)n1] | 1.85 | 494.118 | 495.24 | A |
| 107 | 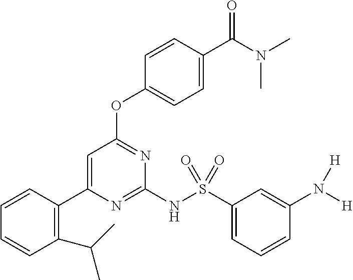 [SMILES: CC(C)c1ccccc1-c1cc(Oc2ccc(C(=O)N(C)C)cc2)nc(NS(=O)(=O)c2cccc(N)c2)n1] | 1.57 | 531.194 | 532.32 | A |
| 108 | 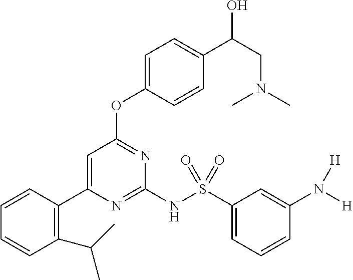 [SMILES: CC(C)c1ccccc1-c1cc(Oc2ccc(C(O)CN(C)C)cc2)nc(NS(=O)(=O)c2cccc(N)c2)n1] | 1.36 | 547.225 | 548.58 | A |
| 109 | 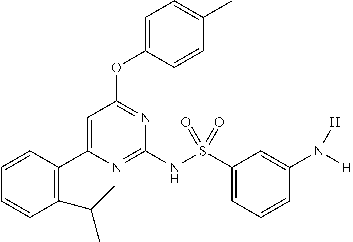 [SMILES: Cc1ccc(Oc2cc(-c3ccccc3C(C)C)nc(NS(=O)(=O)c3cccc(N)c3)n2)cc1] | 1.87 | 474.173 | 475.26 | A |
| 110 | 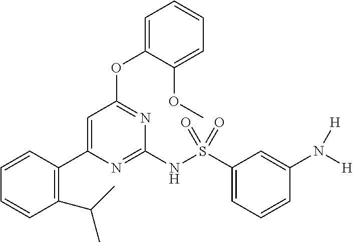 [SMILES: COc1ccccc1Oc1cc(-c2ccccc2C(C)C)nc(NS(=O)(=O)c2cccc(N)c2)n1] | 1.73 | 490.167 | 491.27 | A |
| 111 | 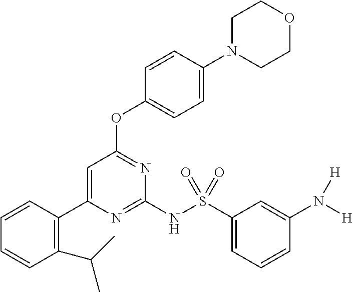 [SMILES: CC(C)c1ccccc1-c1cc(Oc2ccc(N3CCOCC3)cc2)nc(NS(=O)(=O)c2cccc(N)c2)n1] | 1.63 | 545.21 | 546.36 | A |
| 112 | 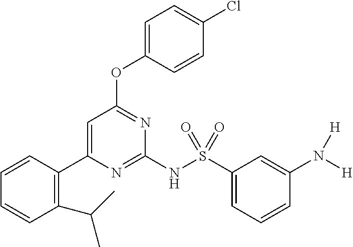 [SMILES: CC(C)c1ccccc1-c1cc(Oc2ccc(Cl)cc2)nc(NS(=O)(=O)c2cccc(N)c2)n1] | 1.95 | 494.118 | 495.24 | A |
| 113 | 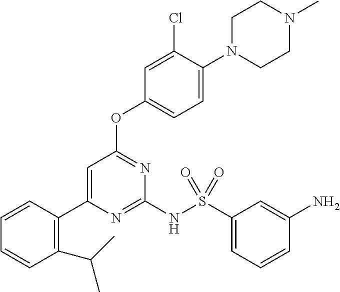 [SMILES: CC(C)c1ccccc1-c1cc(Oc2ccc(N3CCN(C)CC3)c(Cl)c2)nc(NS(=O)(=O)c2cccc(N)c2)n1] | 1.49 | 592.202 | 593.57 | A |
| 114 | 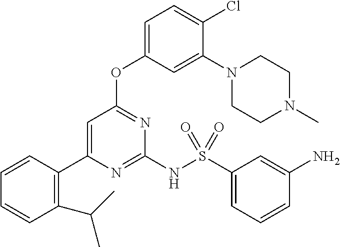 [SMILES: CC(C)c1ccccc1-c1cc(Oc2ccc(Cl)c(N3CCN(C)CC3)c2)nc(NS(=O)(=O)c2cccc(N)c2)n1] | 1.48 | 592.202 | 593.5 | A |
| 115 | 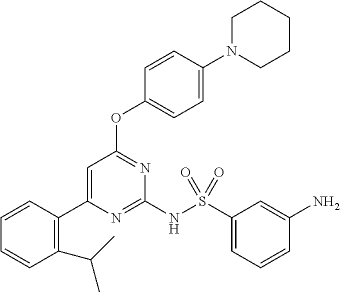 [SMILES: CC(C)c1ccccc1-c1cc(Oc2ccc(N3CCCCC3)cc2)nc(NS(=O)(=O)c2cccc(N)c2)n1] | 1.46 | 543.23 | 544.58 | A |
| 116 | 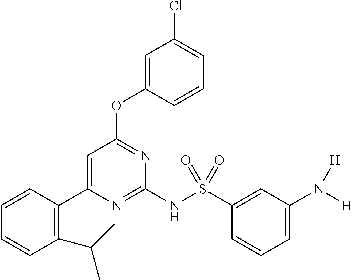 [SMILES: CC(C)c1ccccc1-c1cc(Oc2cccc(Cl)c2)nc(NS(=O)(=O)c2cccc(N)c2)n1] | 2 | 494.118 | 495 | A |
| 117 | 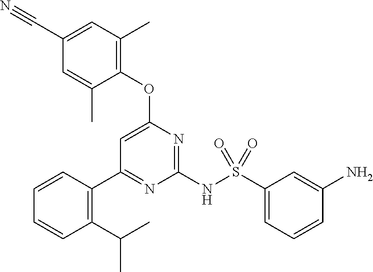 [SMILES: Cc1cc(C#N)cc(C)c1Oc1cc(-c2ccccc2C(C)C)nc(NS(=O)(=O)c2cccc(N)c2)n1] | 1.93 | 513.183 | 514.2 | A |
| 118 | 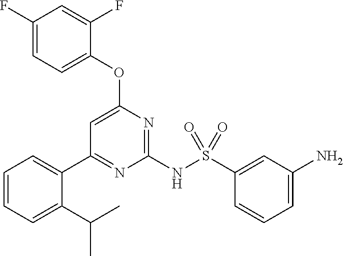 [SMILES: CC(C)c1ccccc1-c1cc(Oc2ccc(F)cc2F)nc(NS(=O)(=O)c2cccc(N)c2)n1] | 1.89 | 496.138 | 497.3 | A |
| 119 | 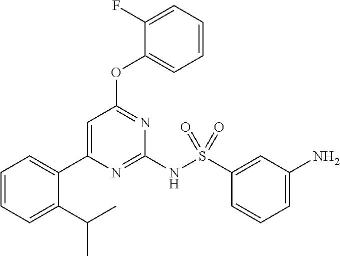 [SMILES: CC(C)c1ccccc1-c1cc(Oc2ccccc2F)nc(NS(=O)(=O)c2cccc(N)c2)n1] | 1.85 | 478.147 | 479.2 | A |
| 120 | 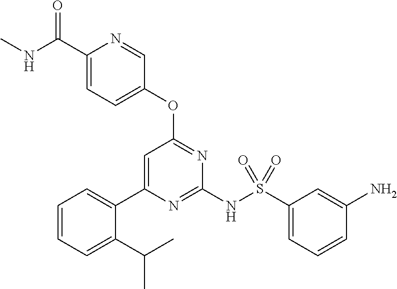 [SMILES: CNC(=O)c1ccc(Oc2cc(-c3ccccc3C(C)C)nc(NS(=O)(=O)c3cccc(N)c3)n2)cn1] | 1.54 | 518.174 | 519.2 | A |

| Compound | |
|---|---|
| number | NMR |
| 85 | ¹H NMR (400 MHz, DMSO-d₆) δ 10.61 (s, 1H), 7.48-7.42 (m, 2H), 7.37 (d, J = |
| | 8.7 Hz, 1H), 7.29-7.23 (m, 1H), 7.16 (dd, J = 8.2, 2.7 Hz, 4H), 7.10-7.01 (m, |
| | 2H), 6.87 (s, 1H), 6.65 (s, 1H), 3.94 (d, J = 10.9 Hz, 3H), 3.52 (d, J = 10.6 Hz, |
| | 3H), 3.25-3.04 (m, 6H), 2.83 (s, 3H), 1.10 (d, J = 6.9 Hz, 6H). |
| 89 | ¹H NMR (400 MHz, DMSO-d₆) δ 10.49 (s, 1H), 10.26 (s, 1H), 8.08 (s, 1H), 7.69 |
| | (d, J = 8.2 Hz, 1H), 7.55-7.42 (m, 3H), 7.30-7.15 (m, 4H), 7.11 (dd, J = 8.1, |
| | 1.3 Hz, 1H), 6.81-6.74 (m, 1H), 6.73 (s, 1H), 3.58-3.50 (m, 5H), 3.33-3.00 |
| | (m, 5H), 2.86 (d, J = 4.5 Hz, 3H), 2.05 (s, 3H), 1.11 (d, J = 6.8 Hz, 6H). |
| 91 | ¹H NMR (400 MHz, DMSO-d₆) δ 8.95 (d, J = 11.1 Hz, 1H), 8.77 (d, J = 11.9 |
| | Hz, 1H), 7.59-7.37 (m, 3H), 7.35-7.07 (m, 6H), 7.06-6.88 (m, 2H), 6.62 (d, J = |
| | 18.3 Hz, 2H), 3.36 (d, J = 12.1 Hz, 2H), 3.21-2.82 (m, 4H), 2.11-1.78 (m, |
| | 4H), 1.11 (d, J = 6.8 Hz, 6H). |
| 92 | ¹H NMR (400 MHz, DMSO-d₆) δ 9.11-8.66 (m, 2H), 7.48-7.43 (m, 2H), 7.40 |
| | (t, J = 2.8 Hz, 3H), 7.32-7.24 (m, 1H), 7.21 (ddd, J = 7.9,5.8, 2.9 Hz, 3H), 7.08 |
| | (t, J = 7.8 Hz, 1H), 6.98 (s, 1H), 6.71 (d, J = 7.6 Hz, 1H), 6.59 (s, 1H), 3.30 (d, |
| | J = 12.4 Hz, 2H), 3.15 (q, J = 6.5 Hz, 1H), 3.02-2.79 (m, 3H), 1.96-1.68 (m, |
| | 4H), 1.11 (d, J = 6.8 Hz, 6H). |
| 93 | ¹H NMR (400 MHz, DMSO-d₆) δ 8.86-8.68 (m, 1H), 8.52-8.30 (m, 1H), 8.19- |
| | 7.98 (m, 1H), 7.94-7.80 (m, 1H), 7.60-7.10 (m, 8H), 6.85 (s, 1H), 3.26-2.87 |
| | (m, 1H), 1.16 (d, J = 15.8, 6.8 Hz, 6H). |
| 103 | ¹H NMR (400 MHz, DMSO-d₆) δ 10.74 (s, 1H), 7.72 (d, J = 8.8 Hz, 1H), 7.67 |
| | (d, J = 2.8 Hz, 1H), 7.60 (dd, J = 8.7, 2.8 Hz, 1H), 7.52-7.40 (m, 2H), 7.34 - |
| | 7.22 (m, 1H), 7.19 (d, J = 7.6 Hz, 1H), 7.06 (s, 1H), 6.94 (dd, J = 7.9 Hz, 1H), |
| | 6.83 (d, J = 8.2 Hz, 1H), 6.69 (s, 1H), 6.46 (d, J = 7.7 Hz, 1H), 3.50 (d, J = 11.4 |
| | Hz, 2H), 3.35-3.22 (m, 2H), 3.22-3.01 (m, 5H), 2.86 (d, J = 3.7 Hz, 3H), 1.12 |
| | (d, J = 6.8 Hz, 6H). |

### Example 34: Preparation of Compound 121

### Step 1: 3-Amino-N-[4-(3-hydroxy-2,2-dimethyl-propoxy)-6-(2-isopropylphenyl)pyrimidin-2-yl]benzenesulfonamide (Compound 121)

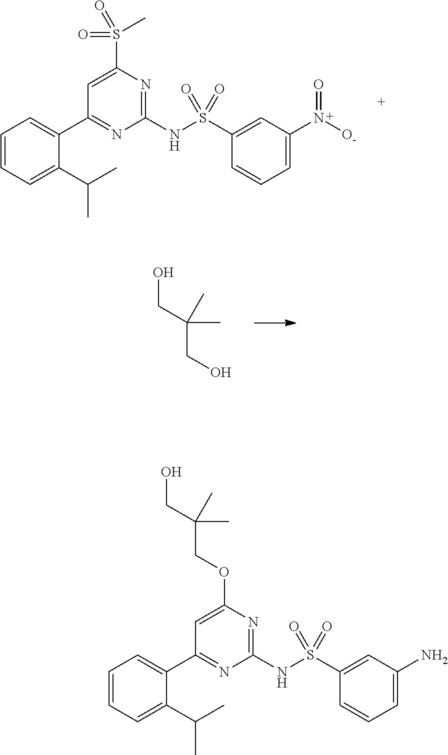

To a 10 mL vial equipped with a magnetic stir bar, N-methylpyrrolidinone (200 μL) and 2,2-dimethylpropane-1,3-diol (10.0 mg, 0.09602 mmol) were added, followed by 60% NaH (5.0 mg, 0.1250 mmol). This slurry was stirred for 5 minutes at room temperature, after which a solution of N-[4-(2-isopropylphenyl)-6-methylsulfonyl-pyrimidin-2-yl]-3-nitro-benzenesulfonamide (9.5 mg, 0.01994 mmol) in N-methylpyrrolidinone (300 μL) was added. After stirring at room temperature for 20 minutes, the reaction was quenched with 1 N HCl (1 mL), and ethyl acetate (3 mL) was added. This mixture was washed with water (2×2 mL) and saturated aqueous sodium chloride solution (2 mL), then dried over sodium sulfate, filtered, and evaporated in vacuo to give 13 mg of crude product, which was carried onto the next stage without purification. To a 10 mL vial equipped with a magnetic stir bar, the crude product from the first step and ethanol (400 μL) were added, and this solution was purged with a balloon of hydrogen gas for 5 minutes. The cap was briefly removed, and palladium on carbon (1.0 mg, 0.009397 mmol) was added. This reaction mixture was stirred under hydrogen gas at room temperature for 1 hour. It was then filtered through Celite and rinsed with ethanol (2 mL). The combined organic solutions were evaporated in vacuo. Purification by reverse phase HPLC (1-99% acetonitrile in water using HCl as a modifier) gave 3-amino-N-[4-(3-hydroxy-2,2-dimethyl-propoxy)-6-(2-isopropylphenyl)pyrimidin-2-yl]benzenesulfonamide (hydrochloride salt) (6.5 mg, 64%). ESI-MS m/z calc. 470.19876, found 471.3 (M+1)⁺; Retention time: 1.51 minutes (LC method A).

### Example 35: Preparation of Compound 122

### Step 1: N-[4-(Cyclopropylmethoxy)-6-(2-isopropylphenyl)pyrimidin-2-yl]-3-nitro-benzenesulfonamide

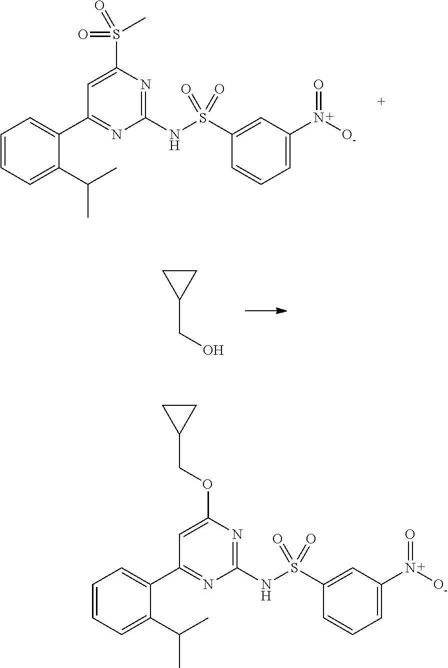

To a solution of cyclopropylmethanol (10 μL) in N-methylpyrrolidinone (200 μL) was added 60% NaH (5.0 mg, 0.1250 mmol) at 0° C. This reaction mixture was stirred for 10 minutes at room temperature. After cooling to 0° C., a solution of N-[4-(2-isopropylphenyl)-6-methylsulfonyl-pyrimidin-2-yl]-3-nitro-benzenesulfonamide (12.7 mg, 0.02665 mmol) in N-methylpyrrolidinone (300 μL) was added, and this reaction mixture was stirred for 10 minutes at room temperature. The reaction mixture was quenched with 1 N HCl (1 mL) and extracted with ethyl acetate (3×1 mL). The combined organic extracts was washed with water (2 mL) and saturated aqueous sodium chloride solution (2 mL), then dried over sodium sulfate, filtered, and evaporated in vacuo to give ˜12 mg of crude product, which contained N-[4-(cyclopropylmethoxy)-6-(2-isopropylphenyl)pyrimidin-2-yl]-3-nitro-benzenesulfonamide. ESI-MS m/z calc. 468.14673, found 469.3 (M+1)⁺; Retention time: 0.71 minutes (LC method D).

### Step 2: 3-Amino-N-[4-(cyclopropylmethoxy)-6-(2-isopropylphenyl)pyrimidin-2-yl]benzenesulfonamide (Compound 122)

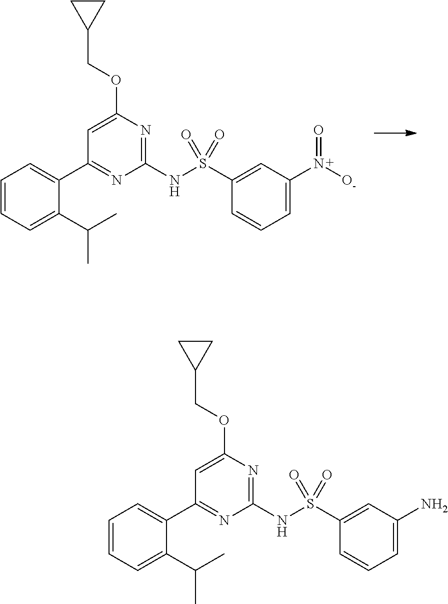

To a 10 mL vial equipped with a magnetic stir bar, a crude product containing N-[4-(cyclopropylmethoxy)-6-(2-isopropylphenyl)pyrimidin-2-yl]-3-nitro-benzenesulfonamide (4 mg, 0.008537 mmol) and ethanol (300 μL) were added, and this solution was purged with a balloon of hydrogen gas for 5 minutes. The cap was briefly removed, and palladium on carbon (1.0 mg, 0.009397 mmol) was added. This reaction mixture was stirred under hydrogen gas at room temperature for 1 hour. It was then filtered through Celite and rinsed with ethanol (2 mL). The combined organic solutions were evaporated in vacuo. Purification by reverse phase HPLC (1-99% acetonitrile in water using HCl as a modifier) gave 3-amino-N-[4-(cyclopropylmethoxy)-6-(2-isopropylphenyl)pyrimidin-2-yl]benzenesulfonamide (hydrochloride salt) (1.4 mg, 35%) ESI-MS m/z calc. 438.17255, found 439.3 (M+1)⁺; Retention time: 1.68 minutes (LC method A).

### Example 36: Characterization of Compounds 123-131

The compounds in the following tables were prepared in a manner analogous to that described above using commercially available reagents and intermediates described herein.

| | | LCMS | | | |
|---|---|---|---|---|---|
| | | Room | | | |
| Compound | | Temperature | Calc. | | LCMS |
| number | Structure | (min) | mass | M + 1 | Method |
| 123 | 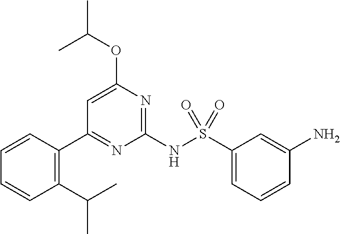 [SMILES: CC(C)Oc1cc(-c2ccccc2C(C)C)nc(NS(=O)(=O)c2cccc(N)c2)n1] | 1.65 | 426.173 | 427.3 | A |
| 124 | 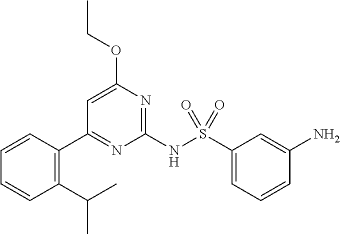 [SMILES: CCOc1cc(-c2ccccc2C(C)C)nc(NS(=O)(=O)c2cccc(N)c2)n1] | 1.54 | 412.157 | 413.2 | A |
| 125 | 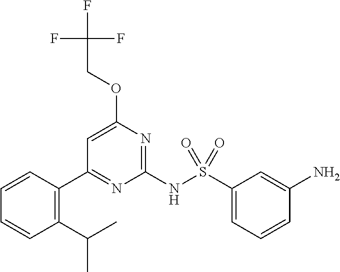 [SMILES: CC(C)c1ccccc1-c1cc(OCC(F)(F)F)nc(NS(=O)(=O)c2cccc(N)c2)n1] | 1.77 | 466.129 | 467.3 | A |
| 126 | 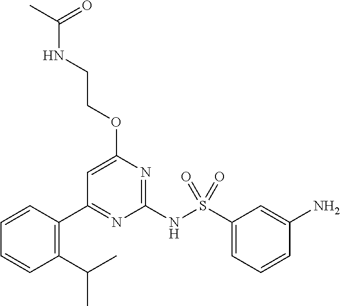 [SMILES: CC(=O)NCCOc1cc(-c2ccccc2C(C)C)nc(NS(=O)(=O)c2cccc(N)c2)n1] | 1.18 | 469.178 | 470.3 | A |
| 127 | 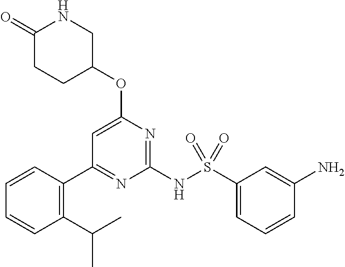 [SMILES: CC(C)c1ccccc1-c1cc(OC2CCC(=O)NC2)nc(NS(=O)(=O)c2cccc(N)c2)n1] | 1.17 | 481.178 | 482.3 | A |
| 128 | 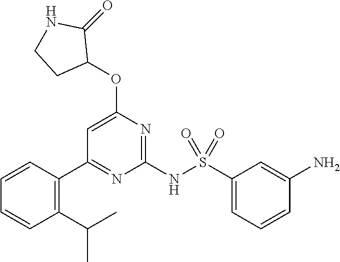 [SMILES: CC(C)c1ccccc1-c1cc(OC2CCNC2=O)nc(NS(=O)(=O)c2cccc(N)c2)n1] | 1.19 | 467.163 | 468.3 | A |
| 129 | 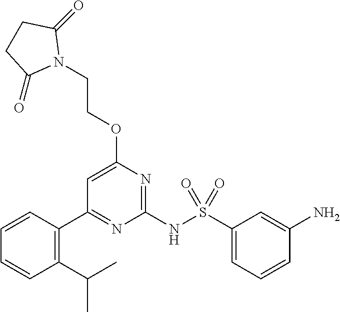 [SMILES: CC(C)c1ccccc1-c1cc(OCCN2C(=O)CCC2=O)nc(NS(=O)(=O)c2cccc(N)c2)n1] | 1.29 | 509.173 | 510.3 | A |
| 130 | 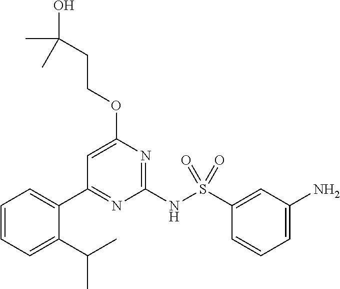 [SMILES: CC(C)c1ccccc1-c1cc(OCCC(C)(C)O)nc(NS(=O)(=O)c2cccc(N)c2)n1] | 1.45 | 470.199 | 471.4 | A |
| 131 | 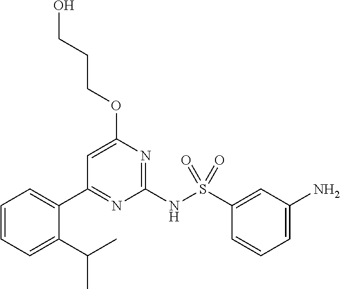 [SMILES: CC(C)c1ccccc1-c1cc(OCCCO)nc(NS(=O)(=O)c2cccc(N)c2)n1] | 1.27 | 442.167 | 443.3 | A |

### Example 37: Preparation of Compound 132

### Step 1: N-[4-(2-chloro-6-methyl-phenoxy)-6-phenyl-pyrimidin-2-yl]benzenesulfonamide (Compound 132)

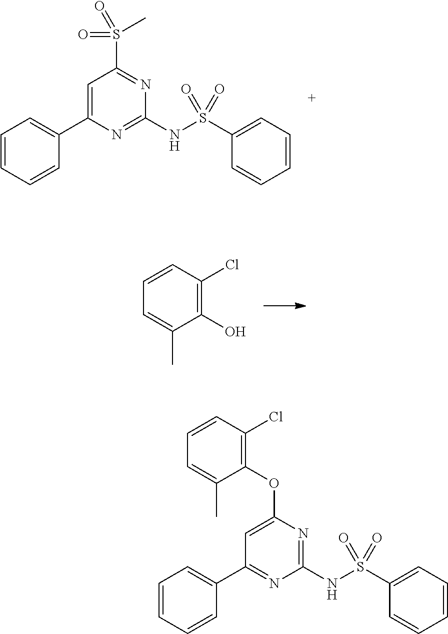

A mixture of N-(4-methylsulfonyl-6-phenyl-pyrimidin-2-yl)benzenesulfonamide (19.47 mg, 0.05 mmol), 2-chloro-6-methyl-phenol (approximately 35.65 mg, 0.2500 mmol) and K₂CO₃ (approximately 34.55 mg, 0.2500 mmol) in NMP (0.5 mL) was stirred at 100° C. for 16 hours. The reaction mixture was cooled, diluted with MeOH, filtered and purification by HPLC (1-99% ACN in water (HCl modifier)) gave N-[4-(2-chloro-6-methyl-phenoxy)-6-phenyl-pyrimidin-2-yl]benzenesulfonamide (9.4 mg, 42%). ESI-MS m/z calc. 451.07574, found 452.2 (M+1)⁺; Retention time: 2.06 minutes; LC method A.

### Example 38: Preparation of Compound 133

### Step 1: N-[4-(1-Isopentylpyrazol-3-yl)oxy-6-phenyl-pyrimidin-2-yl]benzenesulfonamide (Compound 133)

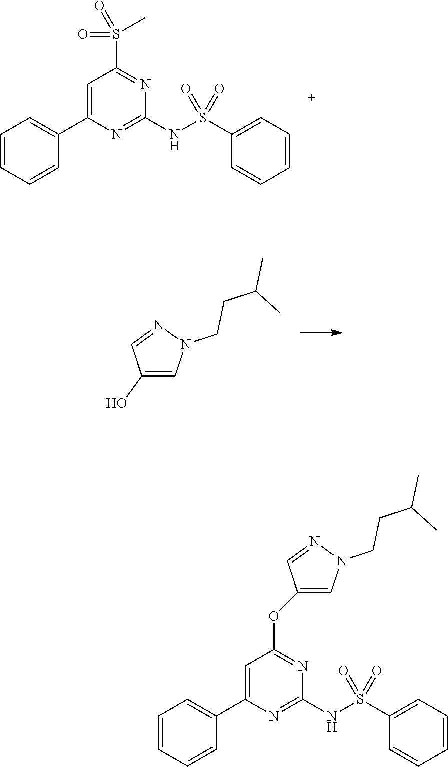 [SMILES: CS(=O)(=O)c1cc(-c2ccccc2)nc(NS(=O)(=O)c2ccccc2)n1 ; CC(C)CCn1cc(O)cn1 ; CC(C)CCn1cc(Oc2cc(-c3ccccc3)nc(NS(=O)(=O)c3ccccc3)n2)cn1]

A mixture of N-(4-methylsulfonyl-6-phenyl-pyrimidin-2-yl)benzenesulfonamide (29.21 mg, 0.075 mmol), 1-isopentylpyrazol-4-ol (approximately 34.70 mg, 0.2250 mmol) and Cs₂CO₃ (approximately 122.2 mg, 0.3750 mmol) in NMP (0.4 mL) was stirred at 80° C. for 90 minutes. The reaction mixture was diluted with MeOH, filtered and purified by HPLC (1-99% ACN in water (HCl modifier)) to give N-[4-(1-isopentylpyrazol-3-yl)oxy-6-phenyl-pyrimidin-2-yl]benzenesulfonamide as a white solid (10.6 mg, 30%). ESI-MS m/z calc. 463.16782, found 464.4 (M+1)⁺; Retention time: 1.95 minutes; LC method A.

### Example 39: Preparation of Compound 134

### Step 1: N-[4-[2-(morpholinomethyl)phenoxy]-6-phenyl-pyrimidin-2-yl]benzenesulfonamide (Compound 134)

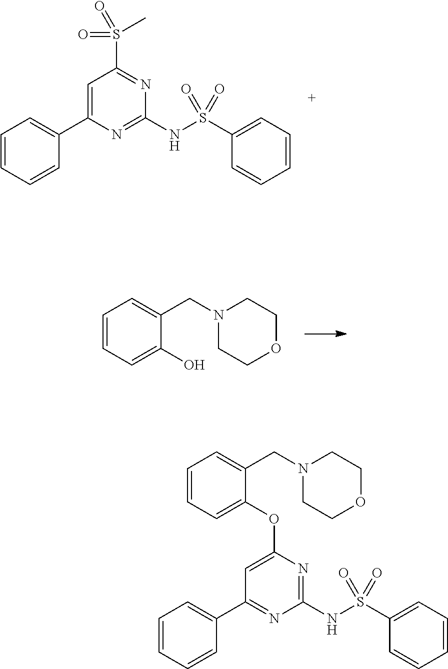 [SMILES: CS(=O)(=O)c1cc(-c2ccccc2)nc(NS(=O)(=O)c2ccccc2)n1 ; Oc1ccccc1CN1CCOCC1 ; O=S(=O)(Nc1nc(Oc2ccccc2CN2CCOCC2)cc(-c2ccccc2)n1)c1ccccc1]

A mixture of N-(4-methylsulfonyl-6-phenyl-pyrimidin-2-yl)benzenesulfonamide (20 mg, 0.05135 mmol), 2-(morpholinomethyl)phenol (approximately 29.76 mg, 0.1540 mmol) and Cs₂CO₃ (approximately 66.92 mg, 0.2054 mmol) in NMP (0.4 mL) was stirred at 80° C. for 16 hours. The reaction mixture was diluted with MeOH, filtered and purified by HPLC (1-99% ACN in water (HCl modifier)) to give N-[4-[2-(morpholinomethyl)phenoxy]-6-phenyl-pyrimidin-2-yl]benzenesulfonamide (hydrochloride salt) (10.1 mg, 39%). ESI-MS m/z calc. 502.16748, found 503.39 (M+1)⁺; Retention time: 0.92 minutes; LC method E.

### Example 40: Preparation of Compound 135

### Step 1: N-(4-Phenoxy-6-phenyl-pyrimidin-2-yl)benzenesulfonamide (Compound 135)

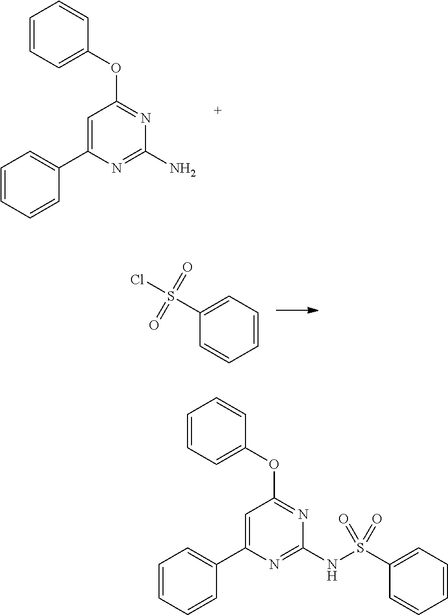

To a solution of 4-phenoxy-6-phenyl-pyrimidin-2-amine (150 mg, 0.5697 mmol) in DMF (3 mL) at 0° C. was added NaH (approximately 68.35 mg of 60% w/w, 1.709 mmol). The mixture was stirred for 10 min at 5° C. It was cooled back to 0° C. and benzenesulfonyl chloride (approximately 150.9 mg, 109.0 μL, 0.8545 mmol) was added. The reaction mixture was stirred at 5° C. for 30 minutes. The reaction mixture was poured in to ice water, and was carefully acidified with 2 N HCl. It was extracted with ethyl acetate. The organic layer was separated, concentrated and was purified by reverse phase HPLC using 10-99% acetonitrile in water using HCl as modifier. It was then purified by silica gel chromatography using only DCM as eluent. Collected the fractions and was concentrated to afford N-(4-phenoxy-6-phenyl-pyrimidin-2-yl)benzenesulfonamide (39.9 mg, 17%). ¹H NMR (400 MHz, Chloroform-d) δ 7.95-7.89 (m, 2H), 7.76-7.69 (m, 2H), 7.56-7.43 (m, 6H), 7.40-7.24 (m, 3H), 7.23-7.16 (m, 2H), 6.89 (s, 1H). ESI-MS m/z calc. 403.09906, found 404.42 (M+1)⁺; Retention time: 1.9 minutes; LC method A.

### Example 41: Preparation of Compound 136

### Step 1: N-[4-(2-Methoxyphenoxy)-6-phenyl-pyrimidin-2-yl]benzenesulfonamide (Compound 136)

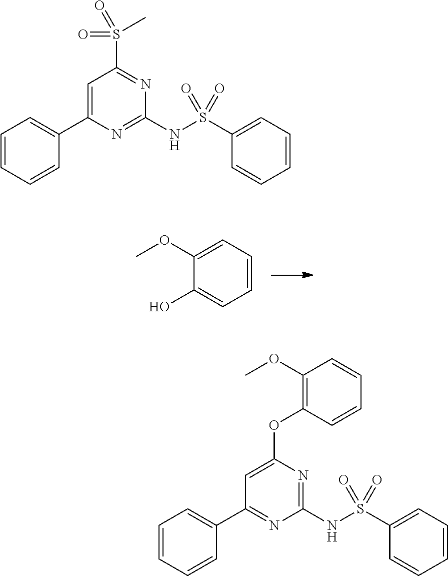

To a mixture of N-(4-methylsulfonyl-6-phenyl-pyrimidin-2-yl)benzenesulfonamide (20 mg, 0.05135 mmol) and 2-methoxyphenol (approximately 12.75 mg, 0.1027 mmol) in NMP (600 μL) was added Potassium carbonate (approximately 28.39 mg, 0.2054 mmol) and heated at 110° C. overnight. It was filtered and the filtrate was purified by reverse phase HPLC using 1-99% acetonitrile in water using HCl as a modifier to afford N-[4-(2-methoxyphenoxy)-6-phenyl-pyrimidin-2-yl]benzenesulfonamide (10.3 mg, 46%)¹H NMR (400 MHz, Chloroform-d) δ 7.97 -7.89 (m, 2H), 7.69-7.61 (m, 2H), 7.53-7.41 (m, 4H), 7.39-7.23 (m, 3H), 7.17-7.03 (m, 3H), 6.94 (s, 1H), 3.80 (s, 3H). ESI-MS m/z calc. 433.10962, found 434.29 (M+1)⁺; Retention time: 1.89 minutes; LC method A.

### Example 42: Preparation of Compound 137

### Step 1: N-[4-(2,2-Dimethylcyclopentoxy)-6-phenyl-pyrimidin-2-yl]benzenesulfonamide (Compound 137)

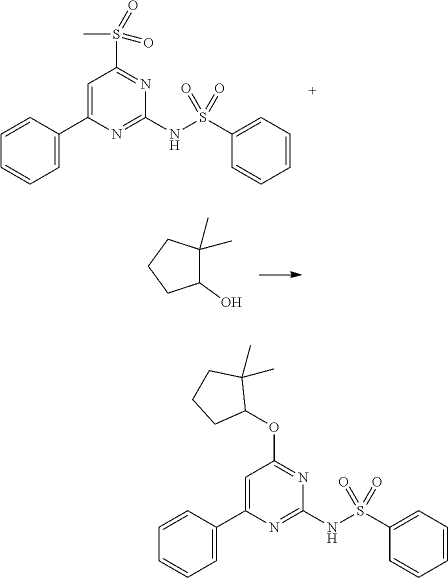

To a solution of N-(4-methylsulfonyl-6-phenyl-pyrimidin-2-yl)benzenesulfonamide (29.21 mg, 0.075 mmol) and 2,2-dimethylcyclopentanol (approximately 42.82 mg, 0.3750 mmol) in NMP (0.4 mL) was added Cs₂CO₃ (approximately 122.2 mg, 0.3750 mmol) and the reaction mixture stirred at 100° C. for 16 hours. The reaction mixture was diluted with MeOH, filtered and purified by HPLC (1-99% ACN in water (HCl modifier)) to give N-[4-(2,2-dimethylcyclopentoxy)-6-phenyl-pyrimidin-2-yl]benzenesulfonamide (10.4 mg, 33%) as a white solid. ESI-MS m/z calc. 423.16165, found 424.5 (M+1)⁺; Retention time: 2.14 minutes; LC method A.

### Example 43: Preparation of Compound 138

### Step 1: N-(4-Norbornan-2-yloxy-6-phenyl-pyrimidin-2-yl)benzenesulfonamide (Compound 138)

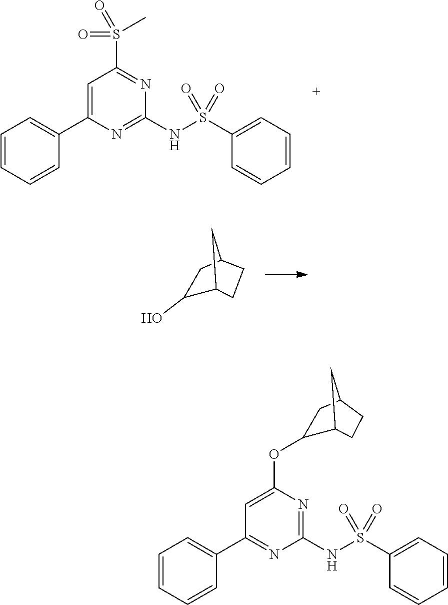 [SMILES: CS(=O)(=O)c1cc(-c2ccccc2)nc(NS(=O)(=O)c2ccccc2)n1 ; OC1CC2CCC1C2 ; O=S(=O)(Nc1nc(OC2CC3CCC2C3)cc(-c2ccccc2)n1)c1ccccc1]

To a solution of norbornan-2-ol (approximately 17.27 mg, 0.1540 mmol) in NMP (250 μL) was added NaH (approximately 10.27 mg of 60% w/w, 0.2568 mmol) at 0° C. and was stirred for 10 minutes. To this mixture was added N-(4-methylsulfonyl-6-phenyl-pyrimidin-2-yl)benzenesulfonamide (20 mg, 0.05135 mmol) in NMP (250 μL) and it was heated at 60° C. for 2 hour. The reaction mixture was quenched with a drop of water. It was filtered and diluted with DMSO and was purified by reverse phase HPLC using 1-99% acetonitrile in water using HCl as modifier to afford N-(4-norbornan-2-yloxy-6-phenyl-pyrimidin-2-yl)benzenesulfonamide (9.6 mg, 44%). ESI-MS m/z calc. 421.14603, found 422.29 (M+1)⁺; Retention time: 2.03 minutes; LC method A.

### Example 44: Preparation of Compound 139

### Step 1: N-(4-Pentoxy-6-phenyl-pyrimidin-2-yl)benzenesulfonamide

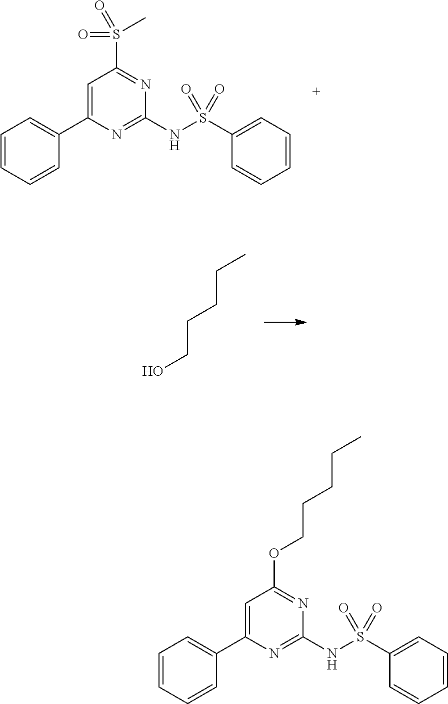

To a solution of pentan-1-ol (approximately 4.526 mg, 5.553 μL, 0.05135 mmol) in NMP (1 mL) was added sodium hydride (approximately 4.929 mg, 5.477 μL, 0.2054 mmol) at 0° C. It was stirred for 10 minutes. To this mixture was added N-(4-methylsulfonyl-6-phenyl-pyrimidin-2-yl)benzenesulfonamide (approximately 20.00 mg, 0.05135 mmol) and was heated to 60° C. for 1 hours. The reaction mixture was quenched with a drop of water, and filtered. DMSO was added and the mixture was purified by reverse phase HPLC using 1-99% acetonitrile in water using HCl as modifier to afford N-(4-pentoxy-6-phenyl-pyrimidin-2-yl)benzenesulfonamide (11.4 mg). ESI-MS m/z calc. 397.14603, found 398.33 (M+1)⁺; Retention time: 2.13 minutes; LC method F.

### Example 45: Preparation of Compound 140

### Step 1: N-[4-(3-Isopropoxypyrazol-1-yl)-6-phenyl-pyrimidin-2-yl]benzenesulfonamide

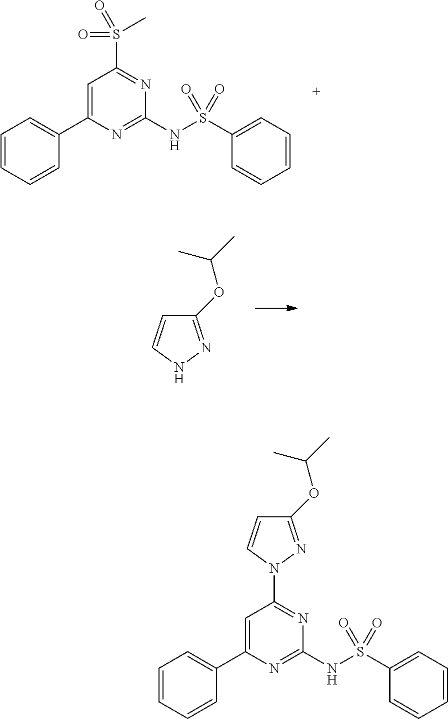

To a solution of 3-isopropoxy-1H-pyrazole (approximately 16.20 mg, 0.1284 mmol) in DMF (1 mL) was added NaH (approximately 10.27 mg of 60% w/w, 0.2568 mmol) at 0° C. and was stirred for 10 min at 0° C. To this mixture was added N-(4-methylsulfonyl-6-phenyl-pyrimidin-2-yl)benzenesulfonamide (25 mg, 0.06419 mmol) and was stirred for 15 min at room temperature. The reaction mixture was quenched with 1 drop of water and was filtered, diluted with DMSO and was purified by reverse phase HPLC using 1-99% acetonitrile in water using HCl as modifier to afford N-[4-(3-isopropoxypyrazol-1-yl)-6-phenyl-pyrimidin-2-yl]benzenesulfonamide (14.5 mg, 52%). ESI-MS m/z calc. 435.1365, found 436.32 (M+1)⁺; Retention time: 2.05 minutes; LC method F.

### Example 46: Preparation of Compound 141

### Step 1: N-[4-Phenyl-6-(1-piperidyl)pyrimidin-2-yl]benzenesulfonamide

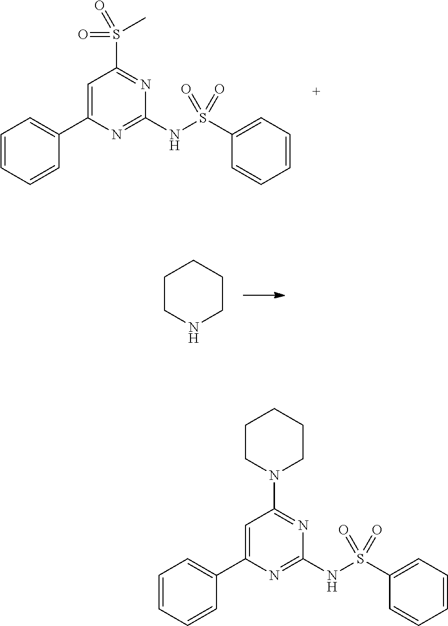 [SMILES: CS(=O)(=O)c1cc(-c2ccccc2)nc(NS(=O)(=O)c2ccccc2)n1 ; C1CCNCC1 ; O=S(=O)(Nc1nc(-c2ccccc2)cc(N2CCCCC2)n1)c1ccccc1]

To a solution of N-(4-methylsulfonyl-6-phenyl-pyrimidin-2-yl)benzenesulfonamide (20 mg, 0.05135 mmol) in NMP (1 mL) was added piperidine (approximately 13.11 mg, 15.23 μL, 0.1540 mmol) and was heated at 100° C. for 30 minutes. The reaction mixture was filtered, diluted with DMSO and was purified by reverse phase HPLC using 1-99% acetonitrile in water using HCl as modifier to afford N-[4-phenyl-6-(1-piperidyl)pyrimidin-2-yl]benzenesulfonamide (14.3 mg, 71%). ESI-MS m/z calc. 394.14633, found 395.31 (M+1)⁺; Retention time: 1.49 minutes; LC method F.

### Example 47: Preparation of Compound 142

### Step 1: N-(4-Phenyl-6-phenylsulfanyl-pyrimidin-2-yl)benzenesulfonamide

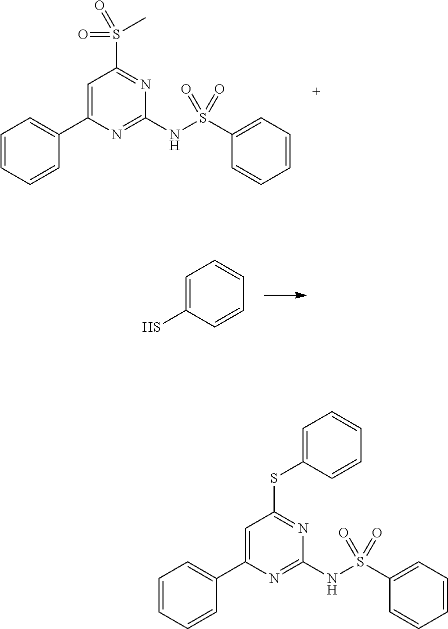 [SMILES: CS(=O)(=O)c1cc(-c2ccccc2)nc(NS(=O)(=O)c2ccccc2)n1 ; Sc1ccccc1 ; O=S(=O)(Nc1nc(Sc2ccccc2)cc(-c2ccccc2)n1)c1ccccc1]

To a solution of N-(4-methylsulfonyl-6-phenyl-pyrimidin-2-yl)benzenesulfonamide (75 mg, 0.1926 mmol) in NMP (1 mL) was added benzenethiol (approximately 25.46 mg, 23.73 μL, 0.2311 mmol) and potassium carbonate (approximately 53.24 mg, 0.3852 mmol) and was heated at 60° C. for 1 hour. Added water and was acidified with 2 N HCl and the product precipitated. It was filtered and was dried to afford N-(4-phenyl-6-phenylsulfanyl-pyrimidin-2-yl)benzenesulfonamide (70 mg, 87%). ESI-MS m/z calc. 419.07623, found 420.25 (M+1)⁺; Retention time: 0.72 minutes; LC method A.

### Example 48: Preparation of Compound 143 and Compound 144

### Step 1: N-[4-(Benzenesulfinyl)-6-phenyl-pyrimidin-2-yl]benzenesulfonamide, (Compound 143) and N-[4-(benzenesulfonyl)-6-phenyl-pyrimidin-2-yl]benzenesulfonamide, (Compound 144)

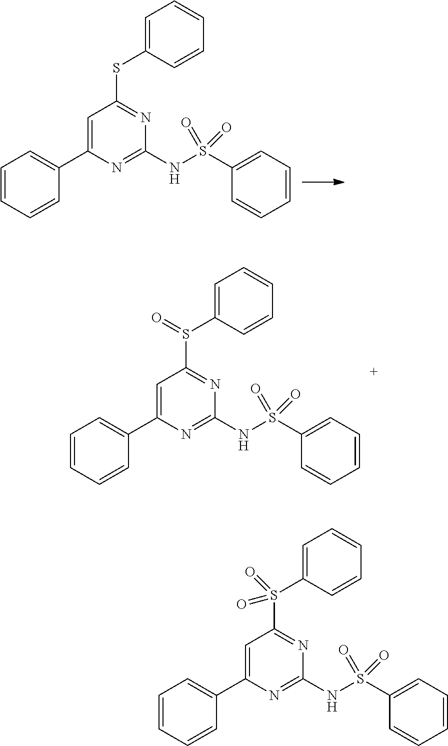

To a solution of N-(4-phenyl-6-phenylsulfanyl-pyrimidin-2-yl)benzenesulfonamide (50 mg, 0.1192 mmol) in methylene chloride (3 mL) was added 3-chloroperoxybenzoic acid (approximately 30.85 mg, 0.1788 mmol) at 0° C. It was stirred at room temperature for 2 hours. Added another 0.5 equivalent of 3-chloro peroxy benzoic acid and was stirred for 30 minutes. It was diluted with sodium thiosulfate and stirred for 10 minutes after which it was extracted with ethyl acetate. The organic layer was separated, dried over Na₂SO₄, concentrated and the residue was dissolved in DMSO and was purified by reverse phase HPLC using 1-99% acetonitrile in water using HCl as modifier to afford N-[4-(benzenesulfinyl)-6-phenyl-pyrimidin-2-yl]benzenesulfonamide (5.9 mg, 11%) ESI-MS m/z calc. 435.07114, found 436.21 (M+1)⁺; Retention time: 1.52 minutes; LC method A; and N-[4-(benzenesulfonyl)-6-phenyl-pyrimidin-2-yl]benzenesulfonamide (13.4 mg, 25%) ESI-MS m/z calc. 451.06604, found 452.25 (M+1)⁺; Retention time: 1.72 minutes; LC method A.

### Example 49: Preparation of Compound 145

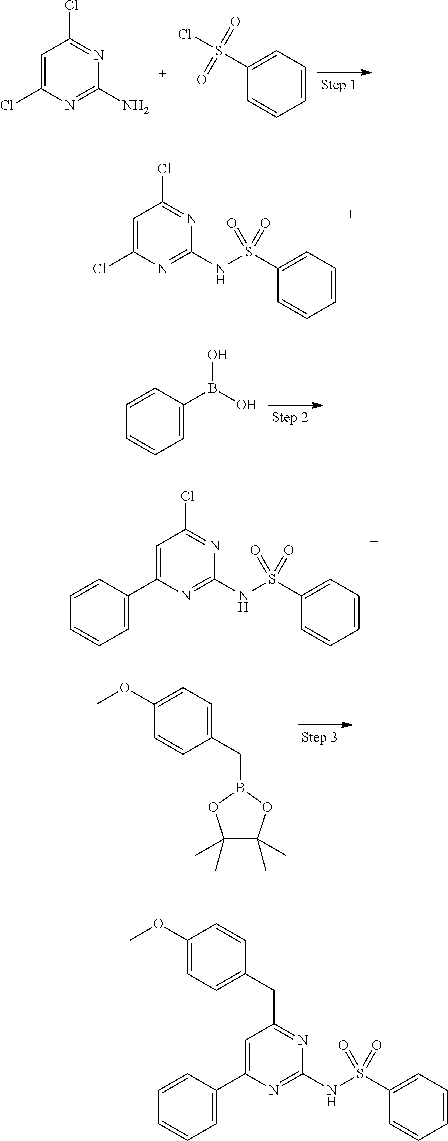

### Step 1: N-(4,6-Dichloropyrimidin-2-yl)benzenesulfonamide

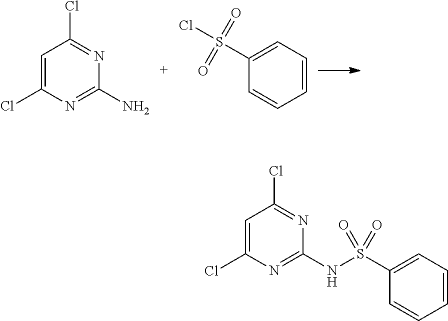

To a solution of 4,6-dichloropyrimidin-2-amine (20 g, 122.0 mmol) in DMF (140.0 mL) was added sodium hydride (approximately 12.20 g of 60% w/w, 305.0 mmol) at 0° C. and stirred for 10 minutes at 0° C. To this mixture was added benzenesulfonyl chloride (approximately 32.32 g, 23.35 mL, 183.0 mmol) in DMF (20.00 mL) at 0° C. very slowly. Stirred the reaction mixture for 20 minutes. The reaction mixture was slowly poured into ice water and was washed 2 times with ethyl acetate (the organic layer was discarded). The aqueous layer was acidified with 2 N HCl (approximately 30 mL) and was extracted multiple times with ethyl acetate. The organic layer was separated, dried over Na₂SO₄, concentrated and the residue was recrystallized using ethyl acetate/hexane to afford pure N-(4,6-dichloropyrimidin-2-yl)benzenesulfonamide (28.2 g, 76%).

### Step 2: N-(4-Chloro-6-phenyl-pyrimidin-2-yl)benzenesulfonamide

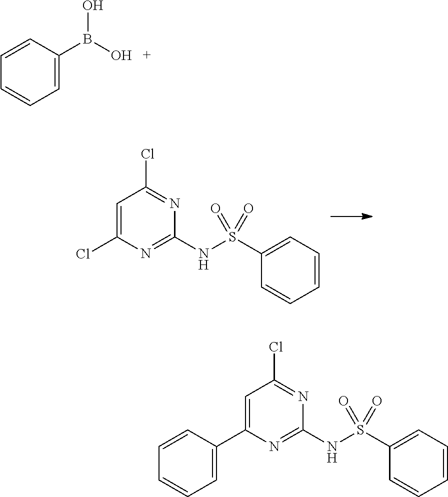

To a solution of N-(4,6-dichloropyrimidin-2-yl)benzenesulfonamide (1 g, 3.288 mmol) and phenylboronic acid (approximately 360.8 mg, 2.959 mmol) in DMF (10 mL), was added potassium carbonate (approximately 6.575 mL of 2 M, 13.15 mmol) and Pd(dppf)C₁₂ (approximately 134.3 mg, 0.1644 mmol). The reaction mixture was flushed with nitrogen. It was heated at 100° C. for 90 minutes. The reaction mixture was added to water and the mixture was acidified with 2 N HCl. The precipitate formed was filtered, which was collected and was purified by reverse phase HPLC using 10-99% acetonitrile in water to afford N-(4-chloro-6-phenyl-pyrimidin-2-yl)benzenesulfonamide (355 mg, 31%) ESI-MS m/z calc. 345.03387, found 346.32 (M+1)⁺; Retention time: 0.66 minutes; LC method D.

### Step 3: N-[4-[(4-Methoxyphenyl)methyl]-6-phenyl-pyrimidin-2-yl]benzenesulfonamide (Compound 145)

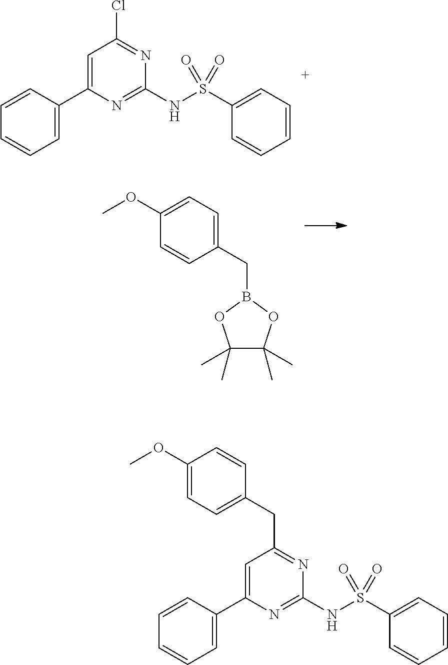

To a solution of N-(4-chloro-6-phenyl-pyrimidin-2-yl)benzenesulfonamide (10 mg, 0.02892 mmol), 2-[(4-methoxyphenyl)methyl]-4,4,5,5-tetramethyl-1,3,2-dioxaborolane (approximately 14.35 mg, 0.05784 mmol) in NMP (1 mL) was added Pd(dppf)C₁₂ (approximately 2.362 mg, 0.002892 mmol) and K₃PO₄ (approximately 18.42 mg, 0.08676 mmol). The reaction mixture was flushed with nitrogen. It was heated at 120° C. overnight. The reaction mixture was filtered and purified by reverse phase HPLC using 1-99% acetonitrile in water using HCl as a modifier to afford N-[4-[(4-methoxyphenyl)methyl]-6-phenyl-pyrimidin-2-yl]benzenesulfonamide (1.2 mg, 10%) ESI-MS m/z calc. 431.13037, found 432.47 (M+1)⁺; Retention time: 1.84 minutes; LC method A.

### Example 50: Preparation of Compound 146

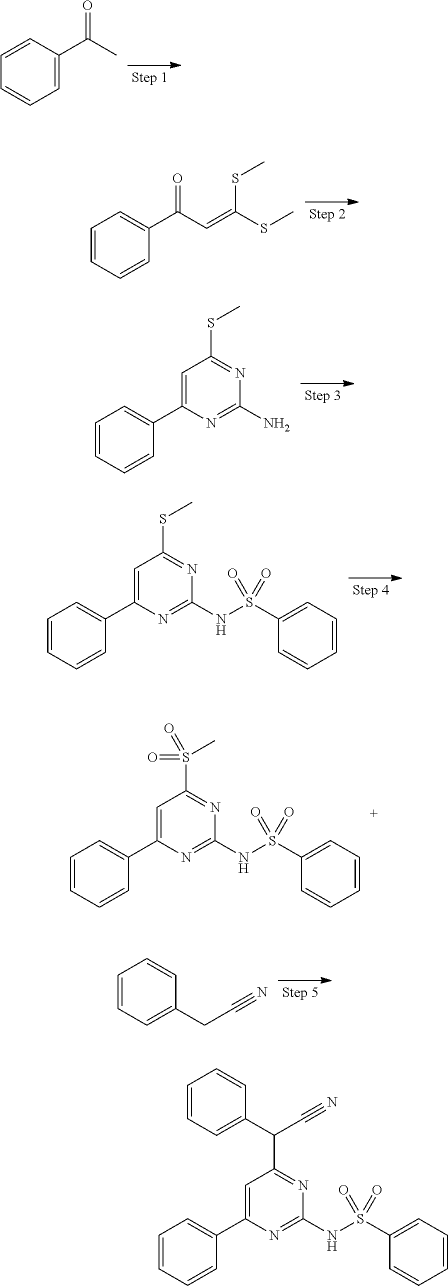

### Step 1: 3,3-Bis(methylsulfanyl)-1-phenyl-prop-2-en-1-one

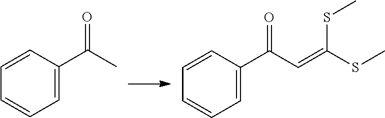

To a solution of 1-phenylethanone (10 g, 83.23 mmol) in tetrahydrofuran (100 mL) was added sodium hydride (approximately 6.659 g of 60% w/w, 166.5 mmol) at 0° C. The mixture was stirred at room temperature for 30 minutes. It was cooled back to 0° C. and carbon disulfide (approximately 6.971 g, 5.506 mL, 91.55 mmol) was added and the reaction was stirred for 30 min at room temperature. Then it was cooled back to 0° C. and iodomethane (approximately 29.54 g, 12.96 mL, 208.1 mmol) was added. The reaction mixture was stirred at room temperature for 3 hours. The reaction mixture was slowly poured into ice cold water and was extracted with ethyl acetate. The organic layer was separated, dried over Na₂SO₄, concentrated and the residue was dissolved in minimum amount of DCM and triturated in hexanes. The product was filtered to afford a yellow solid, 3,3-bis(methylsulfanyl)-1-phenyl-prop-2-en-1-one (12.2 g, 65%). ESI-MS m/z calc. 224.03296, found 225.09 (M+1)⁺; Retention time: 0.58 minutes; LC method D.

### Step 2: 4-Methylsulfanyl-6-phenyl-pyrimidin-2-amine

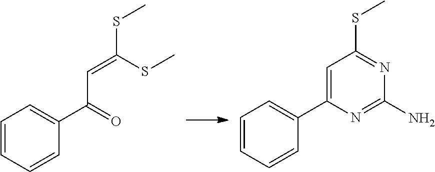

To a solution of 3,3-bis(methylsulfanyl)-1-phenyl-prop-2-en-1-one (4 g, 17.83 mmol) in dimethylformamide (40 mL) was added 3,3-bis(methylsulfanyl)-1-phenyl-prop-2-en-1-one (4 g, 17.83 mmol) and potassium carbonate (approximately 9.857 g, 71.32 mmol). The mixture was heated at 100° C. overnight. The reaction mixture was cooled and water was added. After trituration, the solid was collected by filtration to afford 4-methylsulfanyl-6-phenyl-pyrimidin-2-amine (1.8 g, 46%). ESI-MS m/z calc. 217.06737, found 218.12 (M+1)⁺; Retention time: 0.36 minutes; LC method D.

### Step 3: N-(4-methylsulfanyl-6-phenyl-pyrimidin-2-yl)benzenesulfonamide

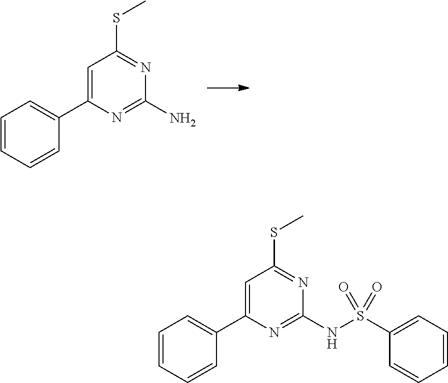 [SMILES: CSc1cc(-c2ccccc2)nc(N)n1 ; CSc1cc(-c2ccccc2)nc(NS(=O)(=O)c2ccccc2)n1]

To a solution of 4-methylsulfanyl-6-phenyl-pyrimidin-2-amine (1.5 g, 6.903 mmol) in DMF (15 mL) at 0° C. was added sodium hydride (approximately 1.104 g of 60% w/w, 27.61 mmol) and the reaction was stirred at rt for 20 minutes. It was cooled back to 0° C. and benzenesulfonyl chloride (approximately 1.828 g, 1.321 mL, 10.35 mmol) in DMF (4 mL) was added and the reaction was stirred at room temperature for 30 minutes. The reaction mixture was poured into ice and was extracted with ethyl acetate. The aqueous layer was neutralized with 2 N HCl (approximately 3 mL) and was extracted with ethyl acetate. The combined organic layer was washed with water (2-3 times) and was separated, dried over Na₂SO₄, concentrated and the residue was dissolved in acetonitrile and partitioned between hexane (to remove mineral oil from NaH), The acetonitrile layer was separated, concentrated and the residue was sonicated with ether, the precipitate was filtered to collect the product, since the filtrate also contains some product it was purified by silica gel chromatography using 0-50% ethyl acetate in hexane to afford N-(4-methylsulfanyl-6-phenyl-pyrimidin-2-yl)benzenesulfonamide (1.3 g, 53%). ESI-MS m/z calc. 357.06058, found 358.2 (M+1)⁺; Retention time: 0.66 minutes; LC method A.

### Step 4: N-(4-Methylsulfonyl-6-phenyl-pyrimidin-2-yl)benzenesulfonamide

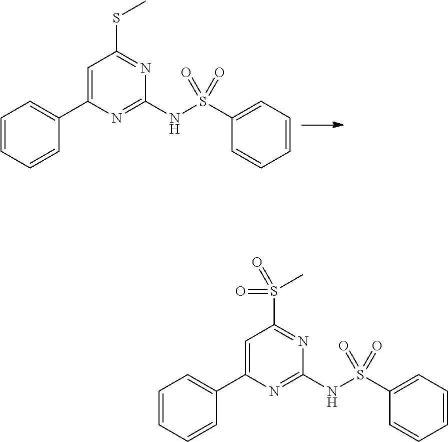 [SMILES: CSc1cc(-c2ccccc2)nc(NS(=O)(=O)c2ccccc2)n1 ; CS(=O)(=O)c1cc(-c2ccccc2)nc(NS(=O)(=O)c2ccccc2)n1]

To a solution of N-(4-methylsulfanyl-6-phenyl-pyrimidin-2-yl)benzenesulfonamide (1.4 g, 3.917 mmol) in methylene chloride (15 mL) was added 3-chloroperoxybenzoic acid (approximately 1.352 g, 7.834 mmol) at 0° C. The reaction mixture was stirred at room temperature for 30 minutes. Added another 1 eq. of m-chloroperoxy benzoic acid was added and the mixture was stirred for 30 minutes. The reaction mixture was diluted sodium thiosulfate and was stirred for 10 minutes. To this mixture was added ethyl acetate. Partitioned the organic layer, dried over Na₂SO₄, concentrated and the residue was recrystallized using ethyl acetate/hexane mixture to afford a white solid, N-(4-methylsulfonyl-6-phenyl-pyrimidin-2-yl)benzenesulfonamide (1.1 g, 72%). ESI-MS m/z calc. 389.05038, found 390.2 (M+1)⁺; Retention time: 0.56 minutes; LC method D.

### Step 5: N-[4-[Cyano(phenyl)methyl]-6-phenyl-pyrimidin-2-yl]benzenesulfonamide (Compound 146)

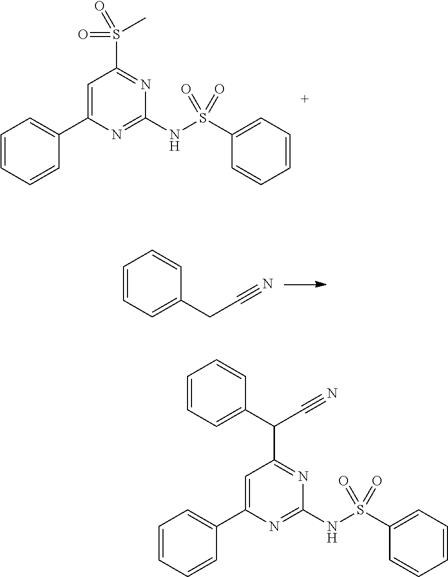

To a solution of 2-phenylacetonitrile (approximately 18.05 mg, 17.78 μL, 0.1541 mmol) in DMF (1 mL) was added NaH (approximately 7.703 mg of 60% w/w, 0.1926 mmol) at 0° C. It was stirred for 20 minutes at room temperature. To this was added N-(4-methylsulfonyl-6-phenyl-pyrimidin-2-yl)benzenesulfonamide (15 mg, 0.03852 mmol) at 0° C. The reaction mixture was stirred at room temperature for 30 minutes. It was neutralized with 2 drops of water. The reaction mixture was filtered, diluted with DMSO and was purified by reverse phase HPLC using 1-99% acetonitrile in water using HCl as modifier to afford N-[4-[cyano(phenyl)methyl]-6-phenyl-pyrimidin-2-yl]benzenesulfonamide ESI-MS m/z calc. 426.11505, found 427.3 (M+1)⁺; Retention time: 7.41 minutes. Final purity was determined by reversed phase UPLC using an Acquity UPLC BEH C₁₈ column (100×2.1 mm, 1.7 m particle) made by Waters (pn: 186002352), and a dual gradient run from 1-99% mobile phase B over 12.98 minutes, held at 99% B for 1 minute, then return to initial conditions. Mobile phase A=H₂O (0.05% CF₃CO₂H). Mobile phase B═CH₃CN (0.035% CF₃CO₂H). Flow rate=0.7 mL/min, injection volume=2.0 μL, and column temperature=60° C.

### Example 51: Characterization of Compounds 147-200

The compounds in the following tables were prepared in a manner analogous to that described above using commercially available reagents and intermediates described herein.

| | | LCMS Room | | | |
|---|---|---|---|---|---|
| Compound | | Temperature | Calc. | | LCMS |
| number | Structure | (min) | mass | M + 1 | Method |
| 147 | 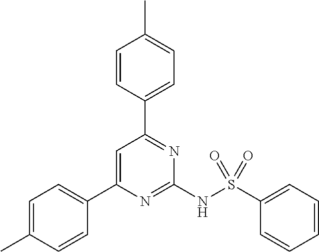 [SMILES: Cc1ccc(-c2cc(-c3ccc(C)cc3)nc(NS(=O)(=O)c3ccccc3)n2)cc1] | 2.08 | 415.135 | 416.2 | A |
| 148 | 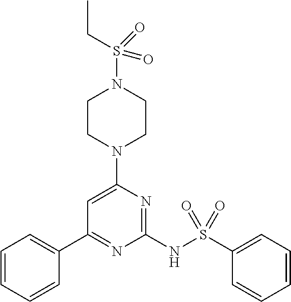 [SMILES: CCS(=O)(=O)N1CCN(c2cc(-c3ccccc3)nc(NS(=O)(=O)c3ccccc3)n2)CC1] | 1.32 | 487.135 | 488.29 | A |
| 149 | 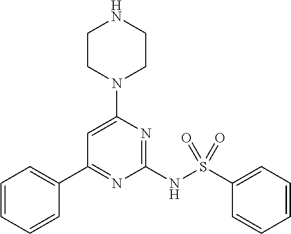 [SMILES: O=S(=O)(Nc1nc(-c2ccccc2)cc(N2CCNCC2)n1)c1ccccc1] | 0.93 | 395.142 | 396.31 | A |
| 150 | 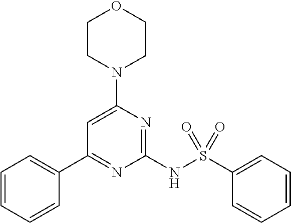 [SMILES: O=S(=O)(Nc1nc(-c2ccccc2)cc(N2CCOCC2)n1)c1ccccc1] | 1.25 | 396.126 | 397.26 | A |
| 151 | 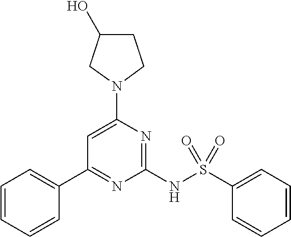 [SMILES: O=S(=O)(Nc1nc(-c2ccccc2)cc(N2CCC(O)C2)n1)c1ccccc1] | 1.17 | 396.126 | 397.26 | A |
| 152 | 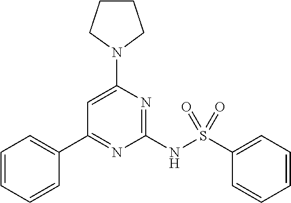 [SMILES: O=S(=O)(Nc1nc(-c2ccccc2)cc(N2CCCC2)n1)c1ccccc1] | 1.42 | 380.131 | 381.26 | A |
| 153 | 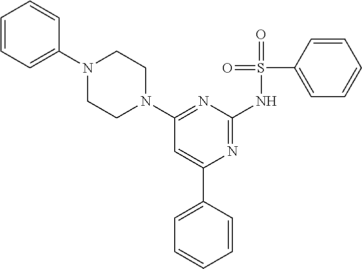 [SMILES: O=S(=O)(Nc1nc(-c2ccccc2)cc(N2CCN(c3ccccc3)CC2)n1)c1ccccc1] | 1.6 | 471.173 | 472 | A |
| 154 | 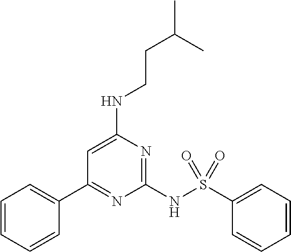 [SMILES: CC(C)CCNc1cc(-c2ccccc2)nc(NS(=O)(=O)c2ccccc2)n1] | 1.56 | 396.162 | 397.34 | A |
| 155 | 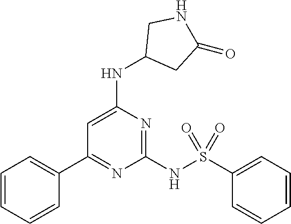 [SMILES: O=C1CC(Nc2cc(-c3ccccc3)nc(NS(=O)(=O)c3ccccc3)n2)CN1] | 1.07 | 409.121 | 410.28 | A |
| 156 | 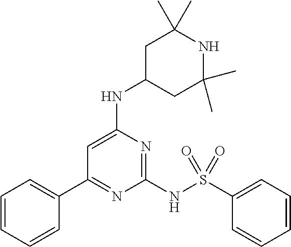 [SMILES: CC1(C)CC(Nc2cc(-c3ccccc3)nc(NS(=O)(=O)c3ccccc3)n2)CC(C)(C)N1] | 1.12 | 465.22 | 466.39 | A |
| 157 | 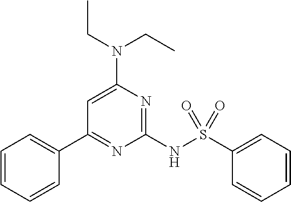 [SMILES: CCN(CC)c1cc(-c2ccccc2)nc(NS(=O)(=O)c2ccccc2)n1] | 1.45 | 382.146 | 383.29 | A |
| 158 | 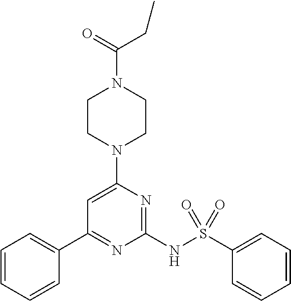 [SMILES: CCC(=O)N1CCN(c2cc(-c3ccccc3)nc(NS(=O)(=O)c3ccccc3)n2)CC1] | 1.25 | 451.168 | 452.32 | A |
| 159 | 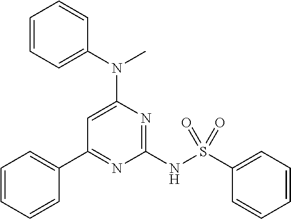 [SMILES: CN(c1ccccc1)c1cc(-c2ccccc2)nc(NS(=O)(=O)c2ccccc2)n1] | 1.62 | 416.131 | 417.28 | A |
| 160 | 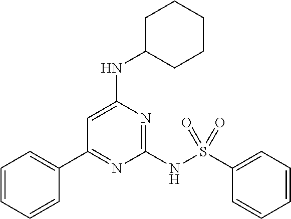 [SMILES: O=S(=O)(Nc1nc(NC2CCCCC2)cc(-c2ccccc2)n1)c1ccccc1] | 1.55 | 408.162 | 409.3 | A |
| 161 | 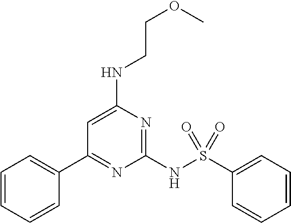 [SMILES: COCCNc1cc(-c2ccccc2)nc(NS(=O)(=O)c2ccccc2)n1] | 1.18 | 384.126 | 385.29 | A |
| 162 | 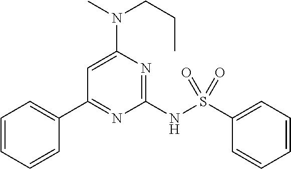 [SMILES: CCCN(C)c1cc(-c2ccccc2)nc(NS(=O)(=O)c2ccccc2)n1] | 1.47 | 382.146 | 383.29 | A |
| 163 | 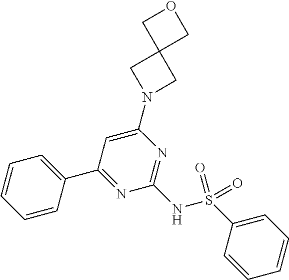 [SMILES: O=S(=O)(Nc1nc(-c2ccccc2)cc(N2CC3(COC3)C2)n1)c1ccccc1] | 1.23 | 408.126 | 409.27 | A |
| 164 | 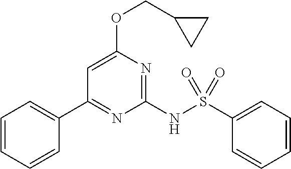 [SMILES: O=S(=O)(Nc1nc(OCC2CC2)cc(-c2ccccc2)n1)c1ccccc1] | 1.77 | 381.115 | 382.28 | A |
| 165 | 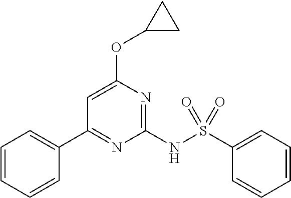 [SMILES: O=S(=O)(Nc1nc(OC2CC2)cc(-c2ccccc2)n1)c1ccccc1] | 1.72 | 367.099 | 368.24 | A |
| 166 | 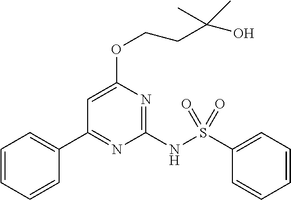 [SMILES: CC(C)(O)CCOc1cc(-c2ccccc2)nc(NS(=O)(=O)c2ccccc2)n1] | 1.55 | 413.141 | 414.31 | A |
| 167 | 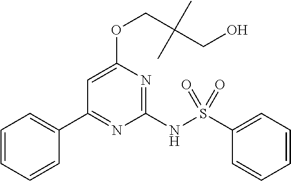 [SMILES: CC(C)(CO)COc1cc(-c2ccccc2)nc(NS(=O)(=O)c2ccccc2)n1] | 1.57 | 413.141 | 414.31 | A |
| 168 | 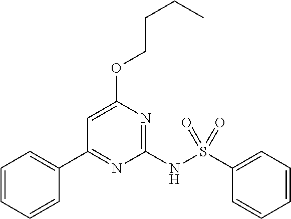 [SMILES: CCCCOc1cc(-c2ccccc2)nc(NS(=O)(=O)c2ccccc2)n1] | 1.9 | 383.13 | 384.27 | A |
| 169 | 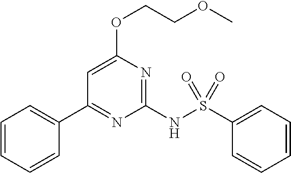 [SMILES: COCCOc1cc(-c2ccccc2)nc(NS(=O)(=O)c2ccccc2)n1] | 1.52 | 385.11 | 386.27 | A |
| 170 | 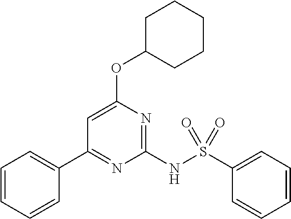 [SMILES: O=S(=O)(Nc1nc(OC2CCCCC2)cc(-c2ccccc2)n1)c1ccccc1] | 2 | 409.146 | 410.32 | A |
| 171 | 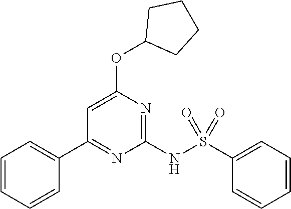 [SMILES: O=S(=O)(Nc1nc(OC2CCCC2)cc(-c2ccccc2)n1)c1ccccc1] | 1.92 | 395.13 | 396.28 | A |
| 172 | 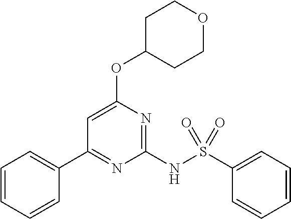 [SMILES: O=S(=O)(Nc1nc(OC2CCOCC2)cc(-c2ccccc2)n1)c1ccccc1] | 1.57 | 411.125 | 412.28 | A |
| 173 | 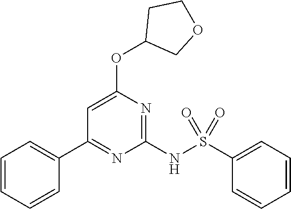 [SMILES: O=S(=O)(Nc1nc(OC2CCOC2)cc(-c2ccccc2)n1)c1ccccc1] | 1.53 | 397.11 | 398.27 | A |
| 174 | 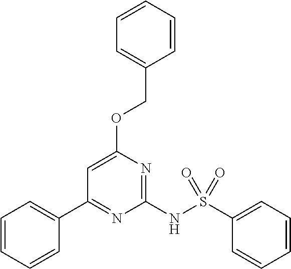 [SMILES: O=S(=O)(Nc1nc(OCc2ccccc2)cc(-c2ccccc2)n1)c1ccccc1] | 1.9 | 417.115 | 418.26 | A |
| 175 | 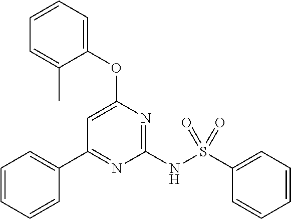 [SMILES: Cc1ccccc1Oc1cc(-c2ccccc2)nc(NS(=O)(=O)c2ccccc2)n1] | 1.95 | 417.115 | 418.26 | A |
| 176 | 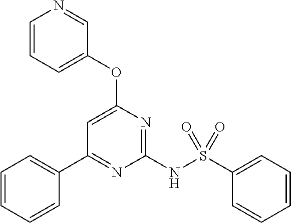 [SMILES: O=S(=O)(Nc1nc(Oc2cccnc2)cc(-c2ccccc2)n1)c1ccccc1] | 1.42 | 404.094 | 405.24 | A |
| 177 | 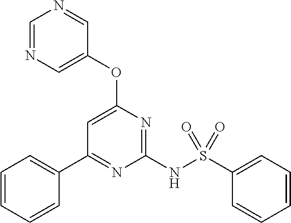 [SMILES: O=S(=O)(Nc1nc(Oc2cncnc2)cc(-c2ccccc2)n1)c1ccccc1] | 1.53 | 405.09 | 406.25 | A |
| 178 | 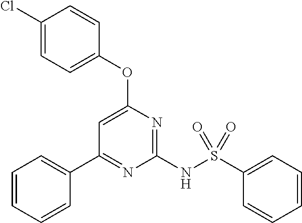 [SMILES: O=S(=O)(Nc1nc(Oc2ccc(Cl)cc2)cc(-c2ccccc2)n1)c1ccccc1] | 2 | 437.06 | 438.21 | A |
| 179 | 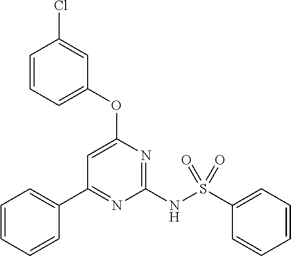 [SMILES: O=S(=O)(Nc1nc(Oc2cccc(Cl)c2)cc(-c2ccccc2)n1)c1ccccc1] | 2.02 | 437.06 | 438.21 | A |
| 180 | 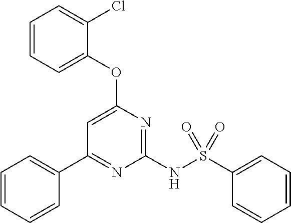 [SMILES: O=S(=O)(Nc1nc(Oc2ccccc2Cl)cc(-c2ccccc2)n1)c1ccccc1] | 1.97 | 437.06 | 438.21 | A |
| 181 | 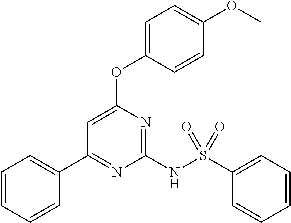 [SMILES: COc1ccc(Oc2cc(-c3ccccc3)nc(NS(=O)(=O)c3ccccc3)n2)cc1] | 1.88 | 433.11 | 434.26 | A |
| 182 | 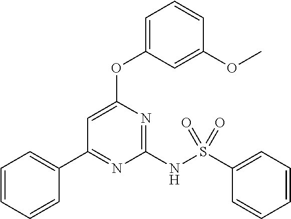 [SMILES: COc1cccc(Oc2cc(-c3ccccc3)nc(NS(=O)(=O)c3ccccc3)n2)c1] | 1.92 | 433.11 | 434.26 | A |
| 183 | 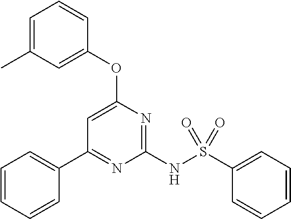 [SMILES: Cc1cccc(Oc2cc(-c3ccccc3)nc(NS(=O)(=O)c3ccccc3)n2)c1] | 2.02 | 417.115 | 418.28 | A |
| 184 | 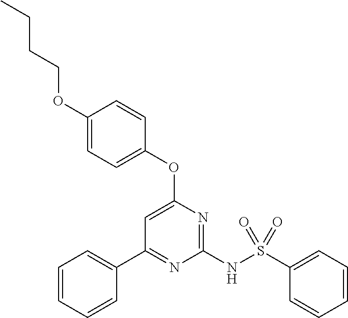 [SMILES: CCCCOc1ccc(Oc2cc(-c3ccccc3)nc(NS(=O)(=O)c3ccccc3)n2)cc1] | 2.18 | 475.157 | 476.32 | A |
| 185 | 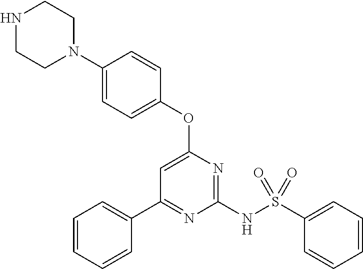 [SMILES: O=S(=O)(Nc1nc(Oc2ccc(N3CCNCC3)cc2)cc(-c2ccccc2)n1)c1ccccc1] | 1.45 | 487.168 | 488.33 | A |
| 186 | 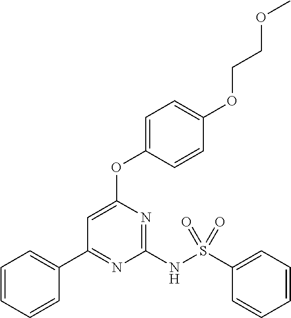 [SMILES: COCCOc1ccc(Oc2cc(-c3ccccc3)nc(NS(=O)(=O)c3ccccc3)n2)cc1] | 1.85 | 477.136 | 478.32 | A |
| 187 | 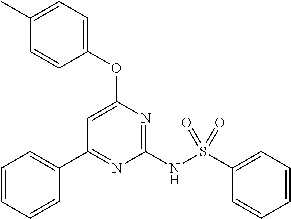 [SMILES: Cc1ccc(Oc2cc(-c3ccccc3)nc(NS(=O)(=O)c3ccccc3)n2)cc1] | 1.98 | 417.115 | 418.26 | A |
| 188 | 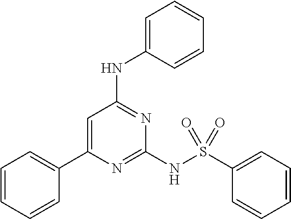 [SMILES: O=S(=O)(Nc1nc(Nc2ccccc2)cc(-c2ccccc2)n1)c1ccccc1] | 1.49 | 402.115 | 403.3 | A |
| 189 | 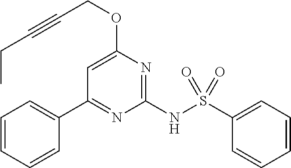 [SMILES: CCC#CCOc1cc(-c2ccccc2)nc(NS(=O)(=O)c2ccccc2)n1] | 1.85 | 393.115 | 394.3 | A |
| 190 | 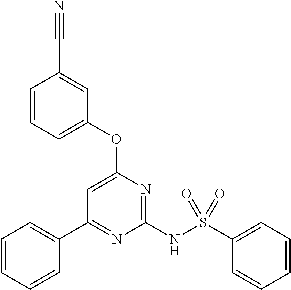 [SMILES: N#Cc1cccc(Oc2cc(-c3ccccc3)nc(NS(=O)(=O)c3ccccc3)n2)c1] | 1.32 | 428.094 | 429.35 | R |
| 191 | 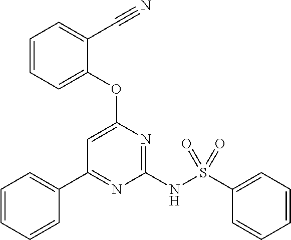 [SMILES: N#Cc1ccccc1Oc1cc(-c2ccccc2)nc(NS(=O)(=O)c2ccccc2)n1] | 1.28 | 428.094 | 429.35 | R |
| 192 | 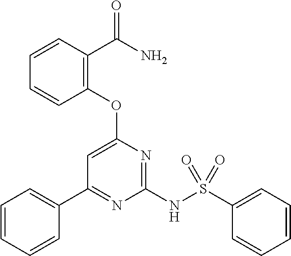 [SMILES: NC(=O)c1ccccc1Oc1cc(-c2ccccc2)nc(NS(=O)(=O)c2ccccc2)n1] | 1.31 | 446.105 | 447.34 | R |
| 193 | 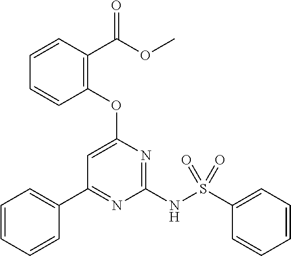 [SMILES: COC(=O)c1ccccc1Oc1cc(-c2ccccc2)nc(NS(=O)(=O)c2ccccc2)n1] | 1.32 | 461.105 | 462.32 | R |
| 194 | 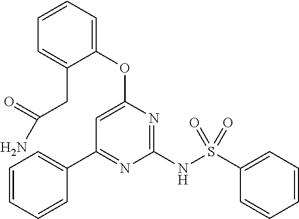 [SMILES: NC(=O)Cc1ccccc1Oc1cc(-c2ccccc2)nc(NS(=O)(=O)c2ccccc2)n1] | 1.07 | 460.121 | 461.34 | R |
| 195 | 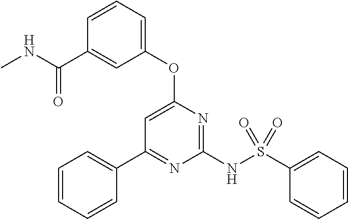 [SMILES: CNC(=O)c1cccc(Oc2cc(-c3ccccc3)nc(NS(=O)(=O)c3ccccc3)n2)c1] | 1.15 | 460.121 | 461.34 | R |
| 196 | 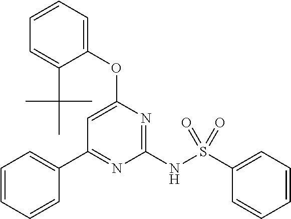 [SMILES: CC(C)(C)c1ccccc1Oc1cc(-c2ccccc2)nc(NS(=O)(=O)c2ccccc2)n1] | 1.6 | 459.162 | 460.36 | R |
| 197 | 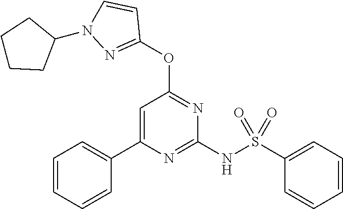 [SMILES: O=S(=O)(Nc1nc(Oc2ccn(C3CCCC3)n2)cc(-c2ccccc2)n1)c1ccccc1] | 1.9 | 461.152 | 462.4 | A |
| 198 | 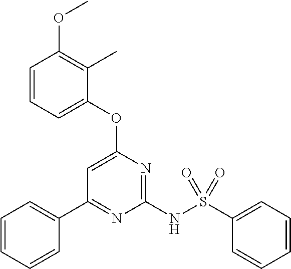 [SMILES: COc1cccc(Oc2cc(-c3ccccc3)nc(NS(=O)(=O)c3ccccc3)n2)c1C] | 2.01 | 447.125 | 448.3 | A |
| 199 | 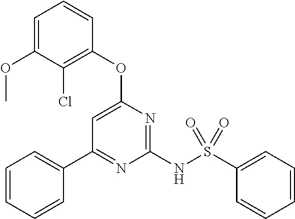 [SMILES: COc1cccc(Oc2cc(-c3ccccc3)nc(NS(=O)(=O)c3ccccc3)n2)c1Cl] | 1.94 | 467.071 | 468.2 | A |
| 200 | 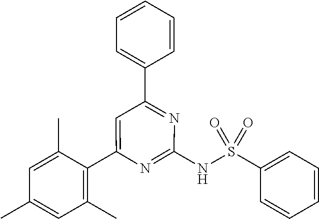 [SMILES: Cc1cc(C)c(-c2cc(-c3ccccc3)nc(NS(=O)(=O)c3ccccc3)n2)c(C)c1] | 2.06 | 429.151 | 430.5 | A |

| Compound | |
|---|---|
| number | NMR |
| 147 | ¹H NMR (400 MHz, DMSO-d₆) δ 11.78 (s, 1H), 8.16-7.98 (m, 7H), 7.68-7.51 |
| | (m, 3H), 7.35 (d, J = 8.3 Hz, 4H), 2.39 (s, 6H). |
| 157 | ¹H NMR (400 MHz, Chloroform-d) δ 8.12-8.05 (m, 2H), 7.79 (dt, J = 7.9, 1.5 |
| | Hz, 2H), 7.60-7.44 (m, 6H), 6.25 (1H), 3.51 (s, 4H), 1.17 (s, 6H). |
| 158 | ¹H NMR (400 MHz, Chloroform-d) δ 8.11-8.04 (m, 2H), 7.93-7.87 (m, 2H), |
| | 7.64-7.47 (m, 5H), 6.47 (1H), 3.96-3.55 (m, 7H), 2.41 (q, J = 7.4 Hz, 2H), 1.20 |
| | (t, J = 7.4 Hz, 3H). |
| 172 | ¹H NMR (400 MHz, Chloroform-d) δ 8.14-8.07 (m, 2H), 7.89 (dd, J = 7.0, 1.8 |
| | Hz, 2H), 7.63-7.56 (m, 1H), 7.56-7.43 (m, 5H), 6.68 (d, J = 2.2 Hz, 1H), 5.09 |
| | (dq, J = 8.5, 4.1 Hz, 1H), 3.95 (dt, J = 11.7, 4.6 Hz, 2H), 3.57 (ddd, J = 11.8, 8.8, |
| | 3.0 Hz, 2H), 1.95 (m, 2H), 1.72 (dtd, J = 12.8, 8.7, 3.9 Hz, 2H). |
| 177 | ¹H NMR (400 MHz, Chloroform-d) δ 9.20 (s, 1H), 8.67 (d, J = 1.2 Hz, 2H), 7.97 |
| | (dt, J = 7.2, 1.2 Hz, 2H), 7.78-7.71 (m, 2H), 7.56-7.46 (m, 4H), 7.38 (td, J = |
| | 8.5, 1.6 Hz, 2H), 7.02 (s, 1H). |

### Example 52: Preparation of Compound 201

### Step 1: 4,6-Bis(2,4,6-trimethylphenyl)pyrimidin-2-amine

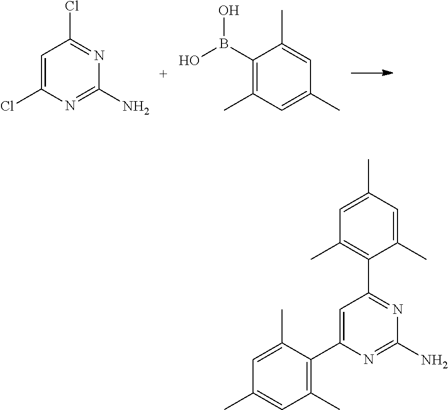

A solution of 4,6-dichloropyrimidin-2-amine (164.0 mg, 1 mmol), (2,4,6-trimethylphenyl)boronic acid (approximately 492.0 mg, 3.000 mmol), potassium carbonate (approximately 2.000 mL of 2 M, 4.000 mmol) and Pd(dppf)C₁₂ (approximately 73.17 mg, 0.1000 mmol) in DME (3.280 mL) was stirred at 90° C. for 4 hours. The reaction mixture was diluted with water and extracted with EtOAc (3×). Organics were combined, dried over sodium sulfate and evaporated to dryness. Purification by column chromatography (24 g silica; 0-50% ethyl acetate in hexanes) gave 4,6-bis(2,4,6-trimethylphenyl)pyrimidin-2-amine (90 mg, 27%) as a white solid. ESI-MS m/z calc. 331.20483, found 332.3 (M+1)⁺; Retention time: 0.67 minutes; LC method D.

### Step 2: N-[4,6-bis(2,4,6-trimethylphenyl)pyrimidin-2-yl]benzenesulfonamide (Compound 201)

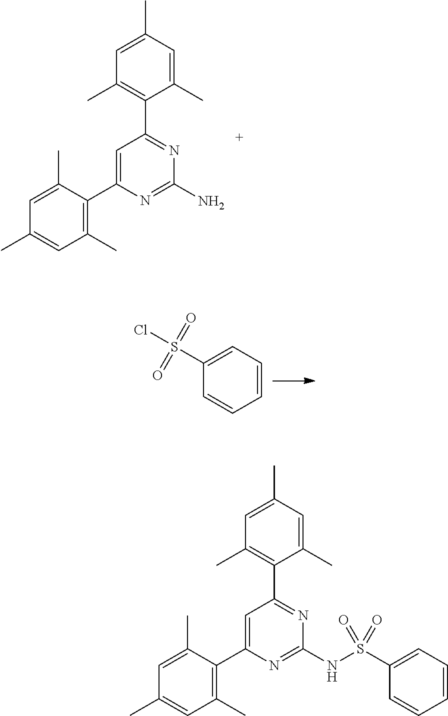 [SMILES: Cc1cc(C)c(-c2cc(-c3c(C)cc(C)cc3C)nc(N)n2)c(C)c1 ; "+" ; O=S(=O)(Cl)c1ccccc1 ; Cc1cc(C)c(-c2cc(-c3c(C)cc(C)cc3C)nc(NS(=O)(=O)c3ccccc3)n2)c(C)c1]

In an open vial. a solution of 4,6-bis(2,4,6-trimethylphenyl)pyrimidin-2-amine (20 mg, 0.06034 mmol) and benzenesulfonyl chloride (200 μL, 1.567 mmol) was heated with a heat gun until the reaction mixture was at reflux. More benzenesulfonyl chloride (200 μL, 1.567 mmol) was added and the reaction mixture heated with a heat gun until the reaction mixture was at reflux. This was done 5 times total. After heating, the residue was taken up in 1:1 DMSO:MeOH and purification by HPLC (1-99% ACN in water (HCl modifier)) gave N-[4,6-bis(2,4,6-trimethylphenyl)pyrimidin-2-yl]benzenesulfonamide (11 mg, 37%) as a white solid. ESI-MS m/z calc. 471.19806, found 472.6 (M+1)⁺; Retention time: 2.0 minutes; LC method A. H NMR (400 MHz, DMSO-d₆) δ 11.81 (s, 1H), 7.86 (d, J=7.9 Hz, 2H), 7.62 (dd, J=7.4 Hz, 1H), 7.48 (dd, J=7.4 Hz, 2H), 7.00-6.87 (m, 5H), 2.27 (s, 6H), 1.89 (s, 12H).

### Example 53: Preparation of Compound 202

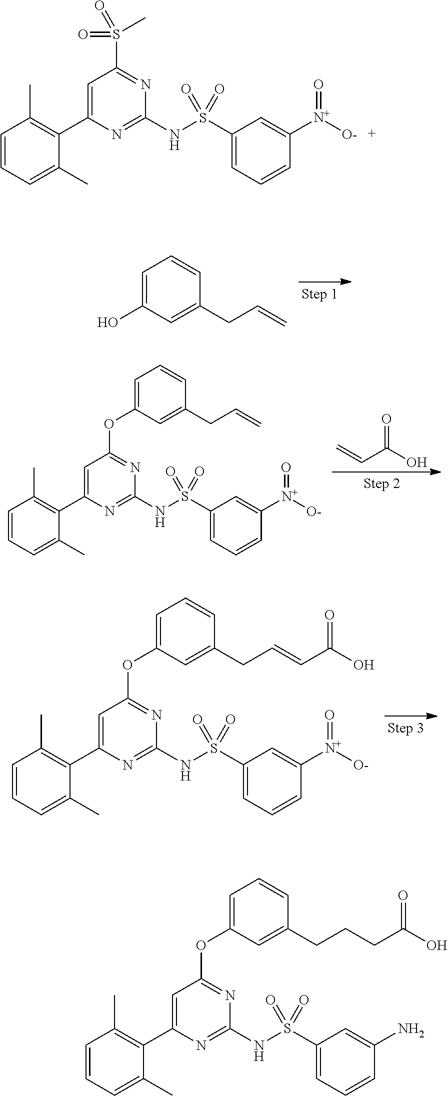

### Step 1: N-[4-(3-allylphenoxy)-6-(2,6-dimethylphenyl)pyrimidin-2-yl]-3-nitro-benzenesulfonamide

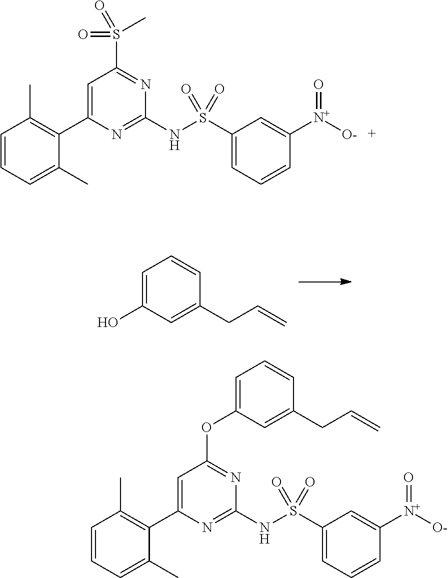

To a 20 mL vial equipped with a magnetic stir bar, N-[4-(2,6-dimethylphenyl)-6-methylsulfonyl-pyrimidin-2-yl]-3-nitro-benzenesulfonamide (0.5031 g, 1.088 mmol), N-methylpyrrolidinone (10.0 mL) and 3-allylphenol (0.5001 g, 3.727 mmol) were added, followed by potassium carbonate (0.5213 g, 3.772 mmol). This solution was stirred at 110° C. for 17 hours. The reaction mixture was then cooled to room temperature, quenched with 1 N HCl (10 mL), and extracted with ethyl acetate (3×15 mL). The combined organic extracts were washed with water (2×25 mL) and saturated aqueous sodium chloride solution (25 mL), then dried over sodium sulfate, filtered, and evaporated in vacuo to give a brown oil. Purification by silica gel chromatography (24 g of silica, 0 to 40% gradient of ethyl acetate/hexanes) gave a white foam, N-[4-(3-allylphenoxy)-6-(2,6-dimethylphenyl)pyrimidin-2-yl]-3-nitro-benzenesulfonamide (342.5 mg, 61%) ESI-MS m/z calc. 516.1467, found 517.3 (M+1)⁺; Retention time: 2.04 minutes; LC method A.

### Step 2: (E)-4-[3-[6-(2,6-dimethylphenyl)-2-[(3-nitrophenyl)sulfonylamino]pyrimidin-4-yl]oxyphenyl]but-2-enoic acid

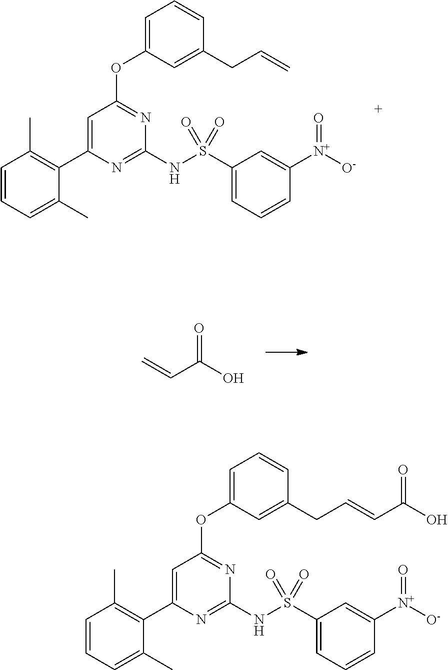

To a 20 mL vial equipped with a magnetic stir bar, N-[4-(3-allylphenoxy)-6-(2,6-dimethylphenyl)pyrimidin-2-yl]-3-nitro-benzenesulfonamide (320.1 mg, 0.6197 mmol), dichloroethane (8.0 mL), and acrylic acid (0.4 mL, 5.834 mmol) were added, followed by Hoveyda-Grubbs 2nd generation catalyst (35.2 mg, 0.05617 mmol). This solution was stirred at 70° C. for 90 minutes. The reaction mixture was then cooled to room temperature, filtered through Celite (rinsed with 10 mL dichloromethane), and evaporated in vacuo. Purification by silica gel chromatography (24 g of silica, 0 to 50% gradient of ethyl acetate/hexanes) gave a yellow foam, (E)-4-[3-[6-(2,6-dimethylphenyl)-2-[(3-nitrophenyl)sulfonylamino]pyrimidin-4-yl]oxyphenyl]but-2-enoic acid (223.4 mg, 51%). ESI-MS m/z calc. 560.1366, found 561.2 (M+1)⁺; Retention time: 1.65 minutes; LC method A.

### Step 3: 4-[3-[2-[(3-aminophenyl)sulfonylamino]-6-(2,6-dimethylphenyl)pyrimidin-4-yl]oxyphenyl]butanoic acid (Compound 202)

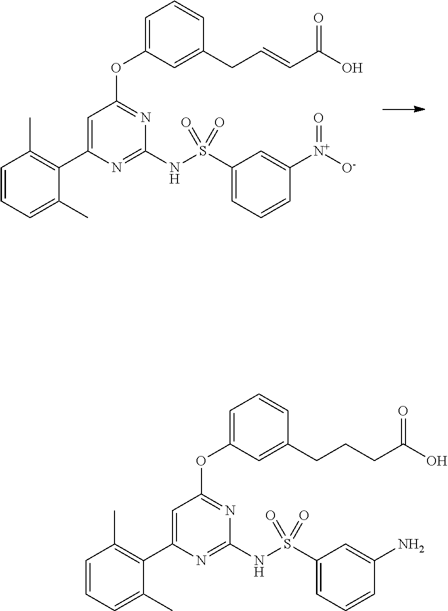 [SMILES: Cc1cccc(C)c1-c1cc(Oc2cccc(C/C=C/C(=O)O)c2)nc(NS(=O)(=O)c2cccc([N+](=O)[O-])c2)n1 ; Cc1cccc(C)c1-c1cc(Oc2cccc(CCCC(=O)O)c2)nc(NS(=O)(=O)c2cccc(N)c2)n1]

In a 10 mL vial equipped with a magnetic stir bar, (E)-4-[3-[6-(2,6-dimethylphenyl)-2-[(3-nitrophenyl)sulfonylamino]pyrimidin-4-yl]oxyphenyl]but-2-enoic acid (101.2 mg, 0.1444 mmol) was dissolved in ethanol (4.0 mL). This solution was sparged with a balloon of hydrogen gas for 5 minutes. The cap was briefly removed, and 10% Pd(OH)₂/C (20.3 mg, 0.01446 mmol) was added. This reaction mixture was stirred under hydrogen gas (2 L, 79.37 mmol) at 60° C. for 17 hours. It was cooled to room temperature, filtered through Celite and rinsed with methanol (6 mL), then evaporated in vacuo. Purification by reverse phase HPLC (1-99% acetonitrile in water using HCl as a modifier) gave the desired 4-[3-[2-[(3-aminophenyl)sulfonylamino]-6-(2,6-dimethylphenyl)pyrimidin-4-yl]oxyphenyl]butanoic acid (hydrochloride salt) (57.6 mg, 70%).

¹H NMR (400 MHz, dimethylsulfoxide-d₆) δ 12.20-11.40 (bs, 1H), 7.42 (t, J=7.8 Hz, 1H), 7.22 (dd, J=8.2, 7.0 Hz, 1H), 7.17 (d, J=7.8 Hz, 1H), 7.14-7.05 (m, 4H), 6.99 (s, 1H), 6.94 (t, J=7.9 Hz, 1H), 6.75 (d, J=8.1 Hz, 1H), 6.62 (d, J=7.7 Hz, 1H), 6.50 (s, 1H), 3.95-3.30 (bs, 2H), 2.65 (d, J=7.7 Hz, 2H), 2.24 (t, J=7.4 Hz, 2H), 1.97 (s, 6H), 1.84 (p, J=7.4 Hz, 2H) ESI-MS m/z calc. 532.17804, found 533.3 (M+1)⁺; Retention time: 1.52 minutes; LC method A.

### Example 54: Preparation of Compound 203

### Step 1: 3-[2-[(3-acetamidophenyl)sulfonylamino]-6-(2,6-dimethylphenyl)pyrimidin-4-yl]oxy-4-chloro-N-methyl-benzamide (Compound 203)

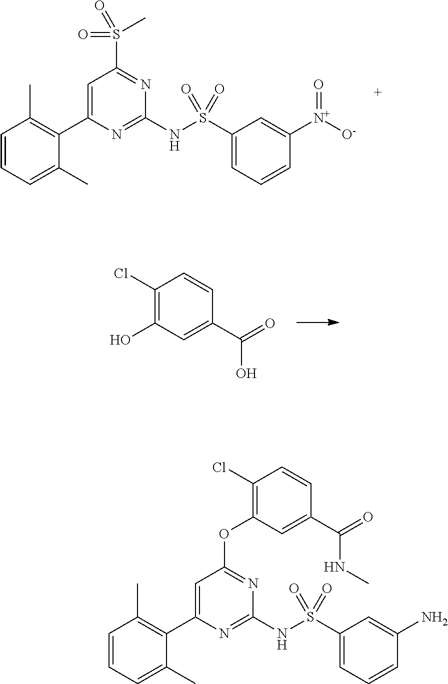 [SMILES: Cc1cccc(C)c1-c1cc(S(C)(=O)=O)nc(NS(=O)(=O)c2cccc([N+](=O)[O-])c2)n1 ; O=C(O)c1ccc(Cl)c(O)c1 ; CNC(=O)c1ccc(Cl)c(Oc2cc(-c3c(C)cccc3C)nc(NS(=O)(=O)c3cccc(N)c3)n2)c1]

Stage 1: To a 20 mL vial equipped with a magnetic stir bar, N-[4-(2,6-dimethylphenyl)-6-methylsulfonyl-pyrimidin-2-yl]-3-nitro-benzenesulfonamide (140.2 mg, 0.3031 mmol), NMP (4.0 mL) and 4-chloro-3-hydroxy-benzoic acid (158.6 mg, 0.9191 mmol) were added, followed by K₂CO₃ (178.5 mg, 1.292 mmol). This solution was stirred at 80° C. for 63 hours. The reaction mixture was then cooled to room temperature, quenched with 1 N HCl (5 mL), and extracted with ethyl acetate (3×5 mL). The combined organic extracts was washed with water (2×6 mL) and saturated aqueous sodium chloride solution (6 mL), then dried over sodium sulfate, filtered, and evaporated in vacuo. This crude product was purified by silica gel chromatography (24 g of silica, 0 to 30% gradient of ethyl acetate/hexanes) to give 107.4 mg of a white solid that was approximately 50% pure.

Stage 2: The product from Stage 1 was partially dissolved in EtOH (3.0 mL) and transferred to a 10 mL microwave vial equipped with a magnetic stir bar. Aqueous HCl (0.5 mL of 1 M, 0.5000 mmol) was added to this slurry, followed by a fine dust of Fe (185.2 mg, 3.316 mmol). This reaction mixture was stirred at 80° C. for 10 minutes. It was cooled to room temperature, filtered through Celite, rinsed with methanol (10 mL), and evaporated in vacuo to give 104.0 mg of a dark brown oil that was approximately 50% pure. 52.0 mg was used for the next reaction, and the remainder was kept.

Stage 3: To a 3 mL vial containing 52.0 mg (0.093 mmol; 0.046 mmol considering its purity) from Stage 2 were added DMF (0.8 mL), DIPEA (50 μL, 0.2871 mmol), MeNH₂ (hydrochloride salt) (25.0 mg, 0.3703 mmol), then HATU (75.6 mg, 0.1988 mmol), in this order. This mixture was stirred at room temperature for 5 minutes, after which it was filtered and purified by reverse phase HPLC (1-70% acetonitrile in water using HCl as a modifier) to give 3-[2-[(3-aminophenyl)sulfonylamino]-6-(2,6-dimethylphenyl)pyrimidin-4-yl]oxy-4-chloro-N-methyl-benzamide (hydrochloride salt) (6.8 mg, 4%) ESI-MS m/z calc. 537.1238, found 538.2 (M+1)⁺; Retention time: 1.46 minutes; LC method A.

### Example 55: Preparation of Compound 204

### Step 1: 3-[2-[(3-Aminophenyl)sulfonylamino]-6-(2,6-dimethylphenyl)pyrimidin-4-yl]oxy-4-chloro-benzoic acid

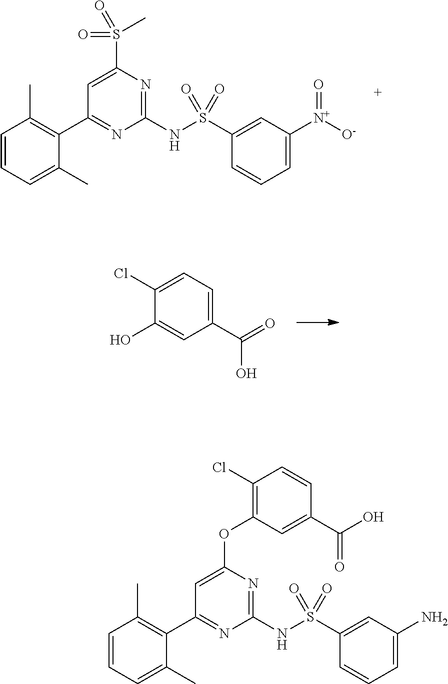 [SMILES: Cc1cccc(C)c1-c1cc(S(C)(=O)=O)nc(NS(=O)(=O)c2cccc([N+](=O)[O-])c2)n1 ; O=C(O)c1ccc(Cl)c(O)c1 ; Cc1cccc(C)c1-c1cc(Oc2cc(C(=O)O)ccc2Cl)nc(NS(=O)(=O)c2cccc(N)c2)n1]

A mixture of N-[4-(2,6-dimethylphenyl)-6-methylsulfonyl-pyrimidin-2-yl]-3-nitro-benzenesulfonamide (160 mg, 0.3459 mmol), 4-chloro-3-hydroxy-benzoic acid (177 mg, 1.026 mmol) and K₂CO₃ (210 mg, 1.519 mmol) in NMP (3 mL) was heated at 110° C. for 16 hours. The reaction mixture was poured into water, the pH adjusted to approximately 3 with iN HCl. The solid was filtered off, washed with water (2×) and dried on the frit. The precipitate was taken up in EtOH (2 mL) and to this was added Fe (188 mg, 3.366 mmol) followed by HCl (approximately 12.61 mg, 0.3459 mmol) and the reaction mixture stirred at 60° C. for 2 hours. The reaction mixture was diluted with EtOH and filtered through Celite. The Celite was washed with water (2×) and EtOH (3×) and then evaporated to dryness to give 3-[2-[(3-aminophenyl)sulfonylamino]-6-(2,6-dimethylphenyl)pyrimidin-4-yl]oxy-4-chloro-benzoic acid (70 mg, 39%) as a reddish solid. The product was used in the next step without further purification. ESI-MS m/z calc. 524.0921, found 525.2 (M+1)⁺; Retention time: 0.62 minutes; LC method D.

### Step 2: 3-Amino-N-[4-[2-chloro-5-(piperidine-1-carbonyl)phenoxy]-6-(2,6-dimethylphenyl)pyrimidin-2-yl]benzenesulfonamide (Compound 204)

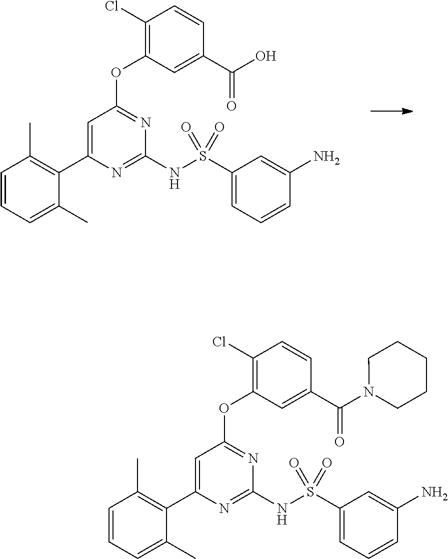 [SMILES: Cc1cccc(C)c1-c1cc(Oc2cc(C(=O)O)ccc2Cl)nc(NS(=O)(=O)c2cccc(N)c2)n1 ; Cc1cccc(C)c1-c1cc(Oc2cc(C(=O)N3CCCCC3)ccc2Cl)nc(NS(=O)(=O)c2cccc(N)c2)n1]

To a 3 mL vial, 3-[2-[(3-aminophenyl)sulfonylamino]-6-(2,6-dimethylphenyl)pyrimidin-4-yl]oxy-4-chloro-benzoic acid (hydrochloride salt) (26.0 mg, 0.04631 mmol), DMF (500 μL), DIPEA (30 μL, 0.1722 mmol), piperidine (50 μL, 0.5056mmol), then HATU (25.2 mg, 0.06628 mmol) were added, in this order. This mixture was stirred at room temperature for 5 minutes, after which it was filtered and purified by reverse phase HPLC (1-70% acetonitrile in water using HCl as a modifier) to give 3-amino-N-[4-[2-chloro-5-(piperidine-1-carbonyl)phenoxy]-6-(2,6-dimethylphenyl)pyrimidin-2-yl]benzenesulfonamide (hydrochloride salt) (1.5 mg, 5%). ESI-MS m/z calc. 591.1707, found 592.3 (M+1)⁺; Retention time: 1.76 minutes; LC method A.

### Example 56: Preparation of Compound 205

### Step 1: N-[3-[[4-(2-chlorophenoxy)-6-(2,6-dimethylphenyl)pyrimidin-2-yl]sulfamoyl]phenyl]acetamide (Compound 205)

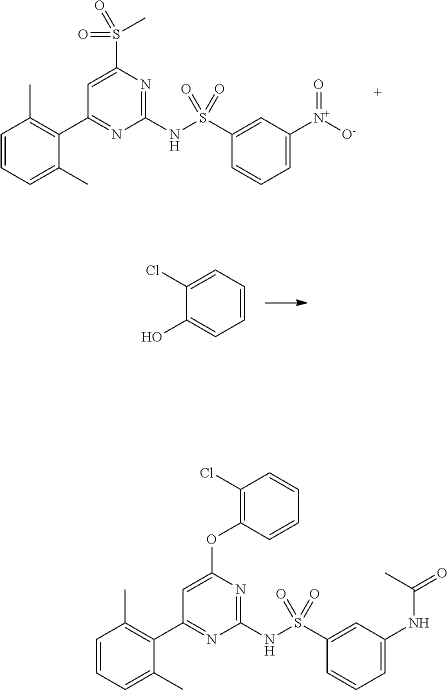 [SMILES: Cc1cccc(C)c1-c1cc(S(C)(=O)=O)nc(NS(=O)(=O)c2cccc([N+](=O)[O-])c2)n1 ; Oc1ccccc1Cl ; CC(=O)Nc1cccc(S(=O)(=O)Nc2nc(Oc3ccccc3Cl)cc(-c3c(C)cccc3C)n2)c1]

Stage 1: To a 20 mL vial equipped with a magnetic stir bar, N-[4-(2,6-dimethylphenyl)-6-methylsulfonyl-pyrimidin-2-yl]-3-nitro-benzenesulfonamide (99.7 mg, 0.2156 mmol), N-methylpyrrolidinone (4.0 mL) and 2-chlorophenol (70 μL, 0.6861 mmol) were added, followed by potassium carbonate (110.3 mg, 0.7981 mmol). This mixture was stirred at 110° C. for 24 hours, after which it was cooled to room temperature, quenched with 1 N HCl (5 mL), and extracted with ethyl acetate (3×5 mL). The combined organic extracts were washed with water (2×5 mL) and saturated aqueous sodium chloride solution (4 mL), then dried over sodium sulfate, filtered, and evaporated in vacuo to give 225 mg of a brown oil. No chromatographic purification was conducted at this stage.

Stage 2: The crude product from Stage 1 was dissolved in ethanol (1.5 mL), to which was added aqueous HCl (0.5 mL of 1.0 M, 0.5000 mmol) and a fine dust of iron (160.8 mg, 2.879 mmol). This reaction mixture was stirred at 70° C. for 20 minutes. It was cooled to room temperature, filtered, and purified by reverse phase HPLC (1-70% acetonitrile in water using HCl as a modifier) to give 3-amino-N-[4-(2-chlorophenoxy)-6-(2,6-dimethylphenyl)pyrimidin-2-yl]benzenesulfonamide (hydrochloride salt) (14.1 mg, 13%). ESI-MS m/z calc. 480.1023, found 481.2 (M+1)⁺; Retention time: 1.74 minutes; LC method A.

Stage 3: The product from Stage 2 was separated into two batches: a 3.9 mg batch, and the remainder (10.2 mg, 0.0197 mmol) for a subsequent reaction. This material was dissolved in dichloromethane (800 μL), to which was added triethylamine (50 μL, 0.3587mmol), acetic anhydride (30 μL, 0.3180 mmol) and 4-dimethylaminopyridine (0.3 mg, 0.002456 mmol). This solution was stirred at room temperature for 100 minutes, after which it was filtered and purified by reverse phase HPLC (1-70% acetonitrile in water using HCl as a modifier) to give N-[3-[[4-(2-chlorophenoxy)-6-(2,6-dimethylphenyl)pyrimidin-2-yl]sulfamoyl]phenyl]acetamide (6 mg, 5%) ESI-MS m/z calc. 522.11285, found 523.2 (M+1)⁺; Retention time: 1.75 minutes; LC method A.

### Example 57: Preparation of Compound 206

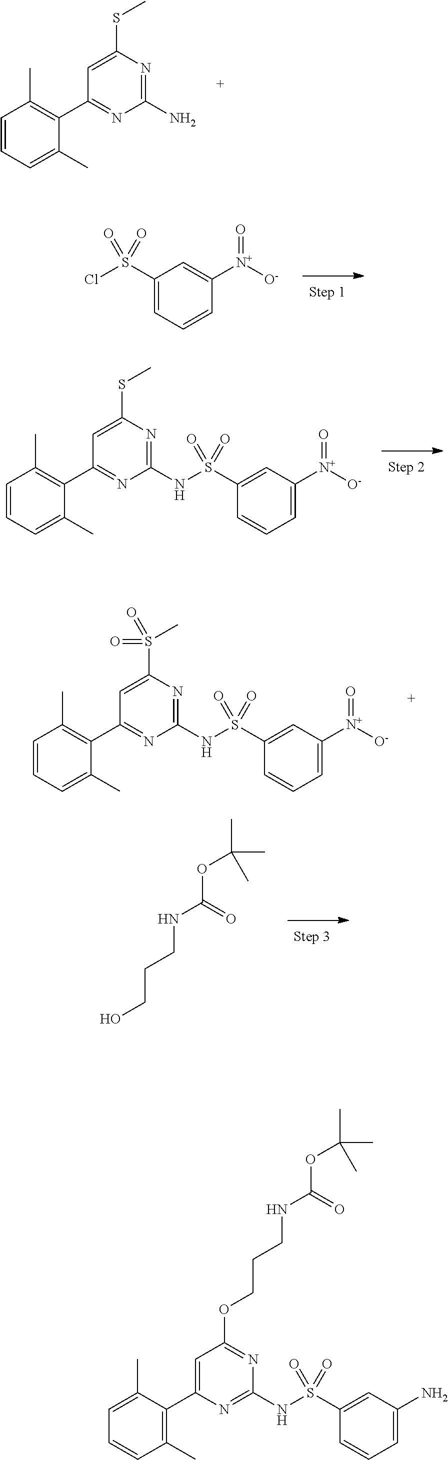 [SMILES: CSc1cc(-c2c(C)cccc2C)nc(N)n1 ; O=[N+]([O-])c1cccc(S(=O)(=O)Cl)c1 ; CSc1cc(-c2c(C)cccc2C)nc(NS(=O)(=O)c2cccc([N+](=O)[O-])c2)n1 ; Cc1cccc(C)c1-c1cc(S(C)(=O)=O)nc(NS(=O)(=O)c2cccc([N+](=O)[O-])c2)n1 ; CC(C)(C)OC(=O)NCCCO ; Cc1cccc(C)c1-c1cc(OCCCNC(=O)OC(C)(C)C)nc(NS(=O)(=O)c2cccc(N)c2)n1]

### Step 1: N-[4-(2,6-dimethylphenyl)-6-methylsulfanyl-pyrimidin-2-yl]-3-nitro-benzenesulfonamide

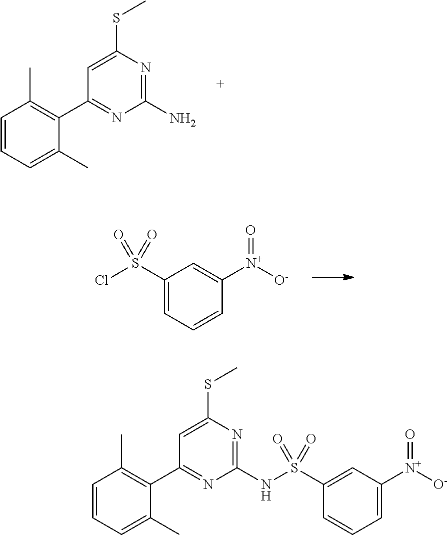 [SMILES: CSc1cc(-c2c(C)cccc2C)nc(N)n1 ; O=[N+]([O-])c1cccc(S(=O)(=O)Cl)c1 ; CSc1cc(-c2c(C)cccc2C)nc(NS(=O)(=O)c2cccc([N+](=O)[O-])c2)n1]

A 250 mL round-bottomed flask equipped with a magnetic stir bar was dried with a heat gun under vacuum and purged with nitrogen; 4-(2,6-dimethylphenyl)-6-methylsulfanyl-pyrimidin-2-amine (6.163 g, 22.61 mmol) and dimethylformamide (80 mL) were added, and this solution was cooled to 0° C. 60% NaH (2.760 g, 69.01 mmol) was added in one portion, and the reaction mixture was warmed to room temperature over 15 minutes. The mixture was cooled to 0° C., upon which 3-nitrobenzenesulfonyl chloride (6.850 g, 30.91 mmol) was added in three portions (CAUTION: hydrogen gas evolution). This solution was stirred at room temperature for 2 hours, then quenched by a slow transfer onto cold 1 N HCl (100 mL). The mixture was extracted with ethyl acetate (3×200 mL). The combined organic extracts were washed with water (2×300 mL) and saturated aqueous sodium chloride solution (300 mL), then dried over sodium sulfate, filtered, and evaporated in vacuo. This crude product was purified by silica gel chromatography (120 g of silica, 0 to 30% gradient of ethyl acetate/hexanes) to give 0.3957 g (6.4%) of recovered starting material, as well as the product, N-[4-(2,6-dimethylphenyl)-6-methylsulfanyl-pyrimidin-2-yl]-3-nitro-benzenesulfonamide (5.50 g, 57%) ESI-MS m/z calc. 430.07693, found 431.1 (M+1)⁺; Retention time: 0.64 minutes; LC method D.

### Step 2: N-[4-(2,6-dimethylphenyl)-6-methylsulfonyl-pyrimidin-2-yl]-3-nitro-benzenesulfonamide

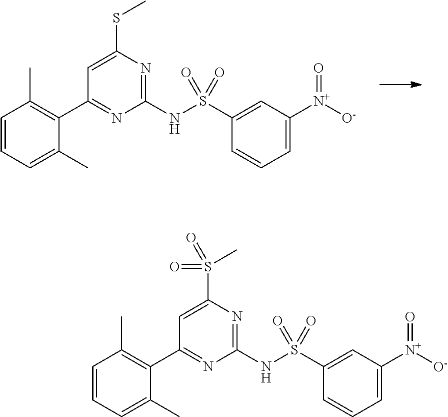 [SMILES: CSc1cc(-c2c(C)cccc2C)nc(NS(=O)(=O)c2cccc([N+](=O)[O-])c2)n1 ; Cc1cccc(C)c1-c1cc(S(C)(=O)=O)nc(NS(=O)(=O)c2cccc([N+](=O)[O-])c2)n1]

To a 250 mL round-bottomed flask equipped with a magnetic stir bar, N-[4-(2,6-dimethylphenyl)-6-methylsulfanyl-pyrimidin-2-yl]-3-nitro-benzenesulfonamide (5.50 g, 12.78 mmol) and dichloromethane (100 mL) were added, followed by 77% m-CPBA (7.56 g, 33.73 mmol). This mixture was stirred at room temperature for 90 minutes, upon which it was quenched with solid sodium thiosulfate (10.36 g, 65.52 mmol). This mixture was stirred for another 90 minutes at room temperature. The reaction mixture was diluted with dichloromethane (300 mL), then washed with water (200 mL), dried over sodium sulfate, filtered, and evaporated in vacuo. This solid was then partially dissolved in dichloromethane (40 mL) and filtered in vacuo on a Buchner funnel to remove the m-chlorobenzoic acid waste. The remaining solution was then purified by silica gel chromatography (120 g of silica, 0 to 60% gradient of ethyl acetate/hexanes) to give N-[4-(2,6-dimethylphenyl)-6-methylsulfonyl-pyrimidin-2-yl]-3-nitro-benzenesulfonamide (4.37 g, 74%) ESI-MS m/z calc. 462.06677, found 463.0 (M+1)⁺; Retention time: 0.61 minutes; LC method D.

### Step 3: tert-Butyl N-[3-[2-[(3-aminophenyl)sulfonylamino]-6-(2,6-dimethylphenyl)pyrimidin-4-yl]oxypropyl]carbamate (Compound 206)

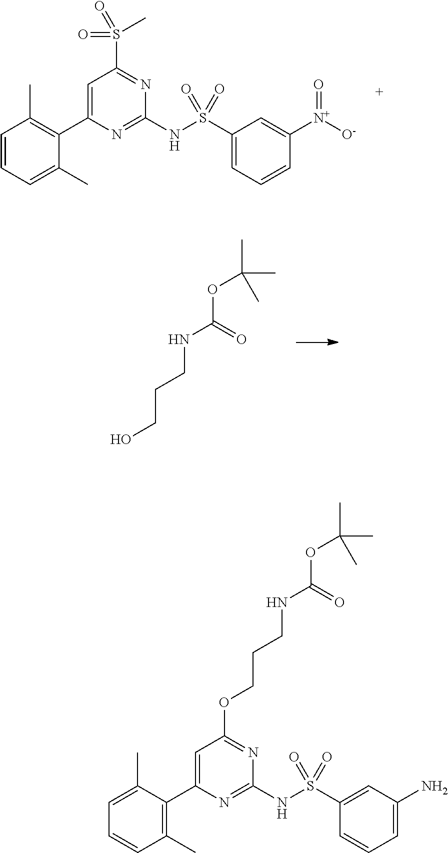 [SMILES: Cc1cccc(C)c1-c1cc(S(C)(=O)=O)nc(NS(=O)(=O)c2cccc([N+](=O)[O-])c2)n1 ; CC(C)(C)OC(=O)NCCCO ; Cc1cccc(C)c1-c1cc(OCCCNC(=O)OC(C)(C)C)nc(NS(=O)(=O)c2cccc(N)c2)n1]

To a 3 mL vial equipped with a magnetic stir bar, NMP (600 μL) and tert-butyl N-(3-hydroxypropyl)carbamate (15.77 mg, 0.090 mmol) were added, followed by 60% NaH (8.000 mg, 0.20003 mmol) (8.0 mg, 0.20 mmol). This slurry was stirred for 5 minutes at room temperature, after which N-[4-(2,6-dimethylphenyl)-6-methylsulfonyl-pyrimidin-2-yl]-3-nitro-benzenesulfonamide (30.00 mg, 0.06163 mmol) (30.0 mg, 0.0616 mmol) was added. After 2 hours at room temperature, the reaction was quenched slowly with 1 N HCl (1 mL), and ethyl acetate (1 mL) was added. After the layers had separated, the aqueous phase was discarded, and the organic layer was washed with water (1 mL). Filtration and purification by reverse phase HPLC (1-99% acetonitrile in water using HCl as a modifier) gave a nitroarene intermediate that was carried to the next step.

The product was dissolved in EtOH (2.0 mL) and transferred to a 10 mL vial equipped with a magnetic stir bar. This solution was sparged with a balloon of H2 for 5 minutes. The cap was briefly removed, and Pd(OH)₂/C (5.0 mg, 0.003560 mmol) was added. This reaction mixture was stirred under a balloon of H2 (2 L, 79.37 mmol) at 60° C. for 2 hours. It was then filtered through Celite and rinsed with MeOH (5 mL), then evaporated in vacuo. Purification by preparative TLC (one full silica plate, 20 cm×20 cm, 250 m thickness, 60 Å particle size, 50% ethyl acetate/hexanes, UV active band) gave tert-butyl N-[3-[2-[(3-aminophenyl)sulfonylamino]-6-(2,6-dimethylphenyl)pyrimidin-4-yl]oxypropyl]carbamate (6.4 mg, 18%). ESI-MS m/z calc. 527.2202, found 528.3 (M+1)⁺; Retention time: 1.58 minutes; LC method A.

### Example 58: Preparation of Compound 207 and Compound 208

### Step 1: N-[4-Chloro-6-(2,6-dimethylphenyl)pyrimidin-2-yl]-3-nitro-benzenesulfonamide

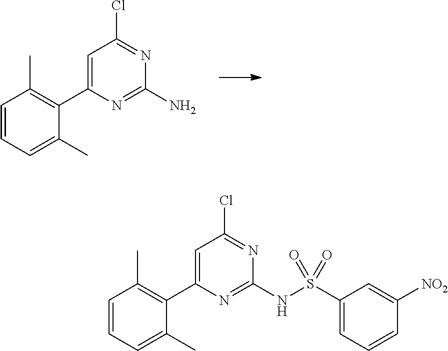 [SMILES: Cc1cccc(C)c1-c1cc(Cl)nc(N)n1 ; Cc1cccc(C)c1-c1cc(Cl)nc(NS(=O)(=O)c2cccc([N+](=O)[O-])c2)n1]

To a suspension of sodium hydride (60% in mineral oil) (4.87 g, 0.122 mol) in anhydrous tetrahydrofuran (30 mL) was added a solution of 4-chloro-6-(2,6-dimethylphenyl)pyrimidin-2-amine (8.13 g, 0.0348 mol) in anhydrous tetrahydrofuran (40 mL) dropwise at 0° C. The reaction mixture was stirred at room temperature for 30 minutes. A solution of 3-nitrobenzenesulfonyl chloride (11.57 g, 52.2 mmol) in anhydrous tetrahydrofuran (40 mL) was added to the reaction mixture dropwise at 0° C. The reaction was stirred at the same temperature for 1 hour. The reaction was quenched with a saturated aqueous solution of sodium bicarbonate (100 mL). The reaction solution was extracted with dichloromethane (3×100 mL). The combined organic layers were washed with water (100 mL), dried over anhydrous sodium sulfate, and then concentrated under vacuum. The residue was purified by silica gel column chromatography using 0 to 10% chloroform-ethyl acetate. The crude product was triturated with a solvent mixture of diethyl ether and hexane (1:5) to furnish N-[4-chloro-6-(2,6-dimethylphenyl)pyrimidin-2-yl]-3-nitro-benzenesulfonamide (5.98 g, 41%) as a white solid. ESI-MS m/z calc. 418.1, found 419.0 (M+1). Retention time: 5.73 minutes. ¹H NMR (250 MHz, CDCl₃) δ (ppm): 9.01 (s, 1H); 8.43 (t, J=10.5 Hz, 2H); 7.682 (t, J=7.8 Hz, 1H); 7.23 (m, 1H); 7.12 (d, J=7.5 Hz, 2H); 6.95 (s, 1H); 1.99 (s, 6H).

### Step 2: 3-amino-N-[4-benzyl-6-(2,6-dimethylphenyl)pyrimidin-2-yl]benzenesulfonamide (Compound 207) and N-[3-[[4-benzyl-6-(2,6-dimethylphenyl)pyrimidin-2-yl]sulfamoyl]phenyl]acetamide (Compound 208)

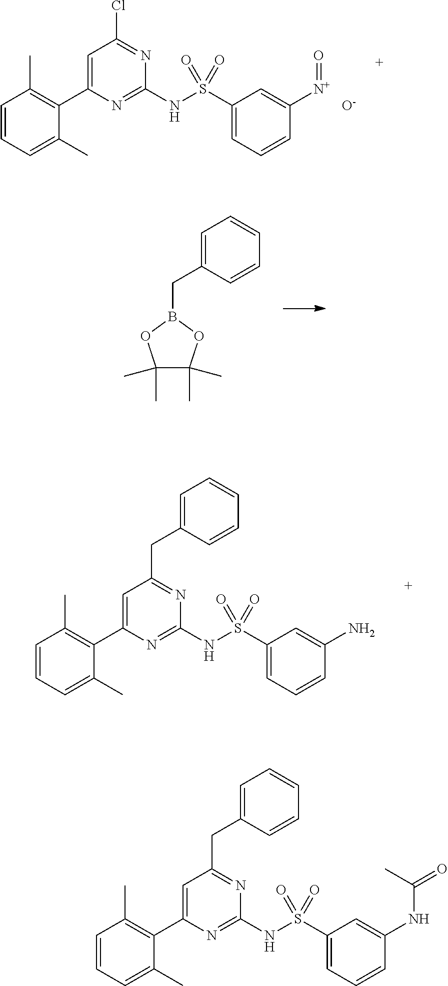

Stage 1: To a 5 mL microwave vial equipped with a magnetic stir bar, N-[4-chloro-6-(2,6-dimethylphenyl)pyrimidin-2-yl]-3-nitro-benzenesulfonamide (60.0 mg, 0.1432 mmol), dioxane (1.0 mL) and benzylboronic acid pinacol ester (59.7 mg, 0.2737 mmol) were added, followed by aqueous sodium carbonate (0.4 mL of 2.0 M, 0.8000 mmol) and Pd(dppf)Cl₂-DCM (10.9 mg, 0.01335 mmol). This mixture was sparged with a balloon of nitrogen gas for 15 minutes under sonication. The reaction mixture was stirred at 70° C. for 19 hours. The reaction mixture was then cooled to room temperature, quenched with 1 N HCl (5 mL), and extracted with ethyl acetate (3×5 mL). The combined organic extracts were washed with water (2×5 mL) and saturated aqueous sodium chloride solution (5 mL), then dried over sodium sulfate, filtered, and evaporated in vacuo. Purification by reverse phase HPLC (1-99% acetonitrile in water using HCl as a modifier) gave two batches of product: a “pure batch” (28.1 mg; 100% pure) and an “impure batch” (16.7 mg; 84% pure).

Stage 2: Each batch from Stage 1 was reacted separately. For the “pure batch”: In a 10 mL microwave vial equipped with a magnetic stir bar, the product from Step 1 was dissolved in EtOH (2.0 mL). This solution was sparged with a balloon of hydrogen gas for 5 minutes. The cap was briefly removed, and 10% Pd(OH)₂/C (5.3 mg, 0.003774 mmol) was added. This reaction mixture was stirred under H2 (2 L, 79.37 mmol) at room temperature for 35 hours, after which it was filtered through Celite and rinsed with methanol (5.0 mL). This solution was evaporated in vacuo, then purified by reverse phase HPLC (1-70% acetonitrile in water using HCl as a modifier) to give 3-amino-N-[4-benzyl-6-(2,6-dimethylphenyl)pyrimidin-2-yl]benzenesulfonamide (11 mg, 17%) ESI-MS m/z calc. 444.162, found 445.3 (M+1)⁺; Retention time: 1.76 minutes; LC method A.

For the “impure batch”: In a 10 mL microwave vial equipped with a magnetic stir bar, the product from Step 1 was dissolved in EtOH (2.0 mL). This solution was sparged with a balloon of hydrogen gas for 5 minutes. The cap was briefly removed, and 10% Pd(OH)₂/C (5.3 mg, 0.003774 mmol) was added. This reaction mixture was stirred under H2 (2 L, 79.37 mmol) at room temperature for 35 hours, after which it was filtered through Celite and rinsed with methanol (5.0 mL). This solution was evaporated in vacuo, then purified by reverse phase HPLC (1-70% acetonitrile in water using HCl as a modifier) to give 3-amino-N-[4-benzyl-6-(2,6-dimethylphenyl)pyrimidin-2-yl]benzenesulfonamide (7.5 mg, 12%), which was carried onto the next step.

Stage 3: The product from the “impure batch” reaction from Stage 2 (7.5 mg, 0.017 mmol) was dissolved in DCM (1.0 mL). Et3N (50 μL, 0.3587 mmol), Ac2O (50 μL, 0.5299 mmol) and DMAP (0.3 mg, 0.002456 mmol) were added in this order. This solution was stirred at room temperature for 30 minutes, after which it was filtered and purified by reverse phase HPLC (1-70% acetonitrile in water using HCl as a modifier) to give N-[3-[[4-benzyl-6-(2,6-dimethylphenyl)pyrimidin-2-yl]sulfamoyl]phenyl]acetamide (4.9 mg, 7%) ESI-MS m/z calc. 486.17255, found 487.3 (M+1)⁺; Retention time: 1.73 minutes; LC method A.

### Example 59: Preparation of Compound 209

### Step 1: N-[4-(2,6-Dimethylphenyl)-6-methylsulfonyl-pyrimidin-2-yl]-3-nitro-benzenesulfonamide

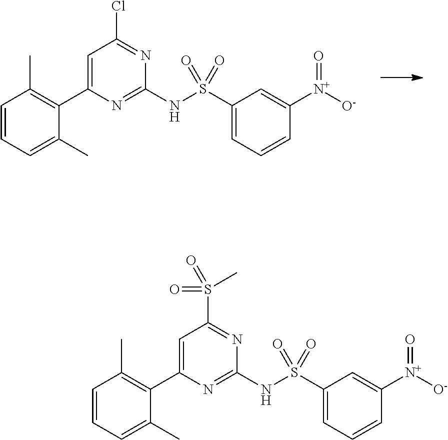 [SMILES: Cc1cccc(C)c1-c1cc(Cl)nc(NS(=O)(=O)c2cccc([N+](=O)[O-])c2)n1 ; Cc1cccc(C)c1-c1cc(S(C)(=O)=O)nc(NS(=O)(=O)c2cccc([N+](=O)[O-])c2)n1]

Stage 1: To a 250 mL round-bottomed flask were added N-[4-chloro-6-(2,6-dimethylphenyl)pyrimidin-2-yl]-3-nitro-benzenesulfonamide (14.14 g, 33.76 mmol), sodium thiomethoxide (5.86 g, 83.61 mmol) and NMP (130 mL). This solution was stirred at 100° C. for 3 hours. The reaction mixture was then cooled to room temperature, quenched with 1 N HCl (300 mL), and extracted with ethyl acetate (3×300 mL). The combined organic extracts were washed with water (300 mL), 3% aqueous hydrogen peroxide solution (300 mL), water (300 mL) and saturated aqueous sodium chloride solution (300 mL), then dried over sodium sulfate, filtered, and evaporated in vacuo. This gave an orange foam (16.71 g, 115% crude product yield) that was carried onto the next reaction.

Stage 2: To a 250 mL round-bottomed flask containing the product from Stage 1, DCM (120 mL) was added, followed by m-CPBA (77% pure, 27.22 g, 121.5 mmol). This solution was stirred at room temperature for 90 minutes. The reaction mixture was quenched by transferring to a 1 L-Erlenmeyer flask containing DCM (400 mL) and solid Na₂S₂O₃ (41.15 g, 260.3 mmol). This mixture was stirred at room temperature for 1 hour. The reaction mixture was diluted with DCM (300 mL), then washed with water (3×400 mL) and saturated aqueous sodium chloride solution (300 mL). The organic layer was then dried over sodium sulfate, filtered, and evaporated in vacuo. This solid was then partially dissolved in DCM (100 mL) and filtered in vacuo on a Buchner funnel to remove the m-chlorobenzoic acid waste (this was repeated three times). The remaining solution was then purified by silica gel chromatography (330 g of silica, 0 to 60% gradient of ethyl acetate/hexanes) to give N-[4-(2,6-dimethylphenyl)-6-methylsulfonyl-pyrimidin-2-yl]-3-nitro-benzenesulfonamide (5.881 g, 36%). ESI-MS m z calc. 462.06677, found 463.1 (M+1)⁺; Retention time: 1.6 minutes; LC method A.

### Step 2: tert-Butyl N-[3-[2-[(3-aminophenyl)sulfonylamino]-6-(2,6-dimethylphenyl)pyrimidin-4-yl]oxycyclohexyl]carbamate (Compound 209)

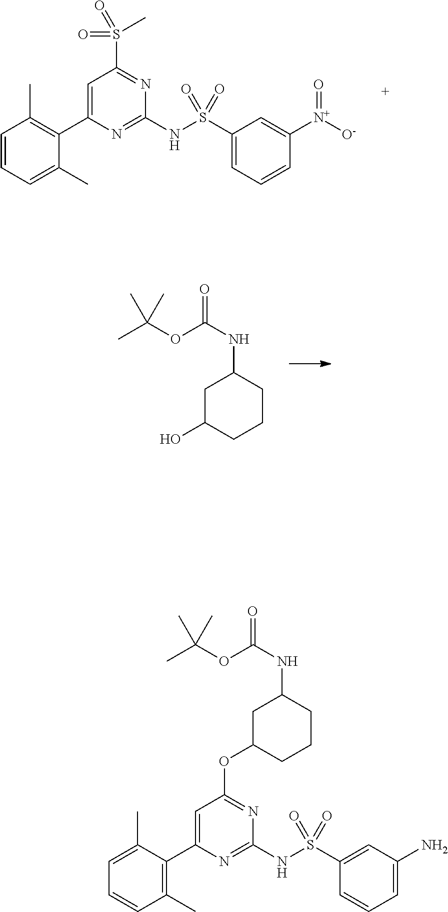 [SMILES: Cc1cccc(C)c1-c1cc(S(C)(=O)=O)nc(NS(=O)(=O)c2cccc([N+](=O)[O-])c2)n1 ; CC(C)(C)OC(=O)NC1CCCC(O)C1 ; Cc1cccc(C)c1-c1cc(OC2CCCC(NC(=O)OC(C)(C)C)C2)nc(NS(=O)(=O)c2cccc(N)c2)n1]

Stage 1: To a 3 mL vial equipped with a magnetic stir bar, NMP (600 μL) and tert-butyl N-(3-hydroxycyClohexyl)carbamate (19.38 mg, 0.090 mmol) were added, followed by 60% NaH (8.000 mg, 0.20003 mmol) (8.0 mg, 0.20 mmol). This slurry was stirred for 5 minutes at room temperature, after which N-[4-(2,6-dimethylphenyl)-6-methylsulfonyl-pyrimidin-2-yl]-3-nitro-benzenesulfonamide (30.00 mg, 0.06163 mmol) (30.0 mg, 0.0616 mmol) was added. After 5 hours at room temperature, the reaction was quenched slowly with 1 N HCl (1 mL), and ethyl acetate (1 mL) was added. After the layers had separated, the aqueous phase was discarded, and the organic layer was washed with water (1 mL). Filtration and purification by reverse phase HPLC (1-99% acetonitrile in water using HCl as a modifier) gave a nitroarene intermediate that was carried to the next step.

Stage 2: The product from Stage 1 was dissolved in EtOH (2.0 mL) and transferred to a 10 mL vial equipped with a magnetic stir bar. This solution was sparged with a balloon of H2 for 5 minutes. The cap was briefly removed, and Pd(OH)₂/C (5.0 mg, 0.003560 mmol) was added. This reaction mixture was stirred under a balloon of H2 (2 L, 79.37 mmol) at 60° C. for 2 hours. It was then filtered through Celite and rinsed with MeOH (5 mL), then evaporated in vacuo. Purification by preparative TLC (one full silica plate, 20 cm×20 cm, 250 m thickness, 60 Å particle size, 50% ethyl acetate/hexanes, UV active band) gave tert-butyl N-[3-[2-[(3-aminophenyl)sulfonylamino]-6-(2,6-dimethylphenyl)pyrimidin-4-yl]oxycyclohexyl]carbamate (9.6 mg, 27%). ESI-MS m/z calc. 567.2515, found 568.3 (M+1)⁺; Retention time: 1.75 minutes; LC method A.

### Example 60: Preparation of Compound 210

### Step 1: 3-Amino-N-[4-(2,6-dimethylphenyl)-6-(2-oxo-3a,4,5,6,7,7a-hexahydro-1,3-benzoxazol-3-yl)pyrimidin-2-yl]benzenesulfonamide (Compound 210)

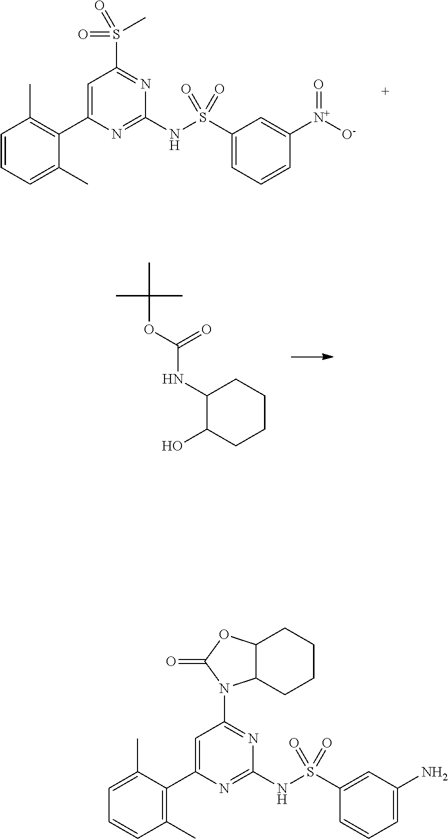 [SMILES: Cc1cccc(C)c1-c1cc(S(C)(=O)=O)nc(NS(=O)(=O)c2cccc([N+](=O)[O-])c2)n1 ; CC(C)(C)OC(=O)NC1CCCCC1O ; Cc1cccc(C)c1-c1cc(N2C(=O)OC3CCCCC32)nc(NS(=O)(=O)c2cccc(N)c2)n1]

Stage 1: To a 3 mL vial equipped with a magnetic stir bar, NMP (600 μL) and tert-butyl N-(2-hydroxycyclohexyl)carbamate (19.38 mg, 0.090 mmol) were added, followed by 60% NaH (8.000 mg, 0.20003 mmol) (8.0 mg, 0.20 mmol). This slurry was stirred for 5 min at room temperature, after which N-[4-(2,6-dimethylphenyl)-6-methylsulfonyl-pyrimidin-2-yl]-3-nitro-benzenesulfonamide (30.00 mg, 0.06163 mmol) (30.0 mg, 0.0616 mmol) was added. After 5 hours at room temperature, the reaction was quenched slowly with 1 N HCl (1 mL), and ethyl acetate (1 mL) was added. After the layers had separated, the aqueous phase was discarded, and the organic layer was washed with water (1 mL). Filtration and purification by reverse phase HPLC (1-99% acetonitrile in water using HCl as a modifier) gave a nitroarene intermediate that was carried to the next step.

Stage 2: The product from Stage 1 was dissolved in EtOH (2.0 mL) and transferred to a 10 mL vial equipped with a magnetic stir bar. This solution was sparged with a balloon of H2 for 5 minutes. The cap was briefly removed, and Pd(OH)₂/C (5.0 mg, 0.003560 mmol) was added. This reaction mixture was stirred under a balloon of H₂ (2 L, 79.37 mmol) at 60° C. for 2hours. It was then filtered through Celite and rinsed with MeOH (5 mL), then evaporated in vacuo. Purification by preparative TLC (one full silica plate, 20 cm×20 cm, 250 μm thickness, 60 Å particle size, 5000 ethyl acetate/hexanes, UV active band) gave 3-amino-N-[4-(2,6-dimethylphenyl)-6-(2-oxo-3 a,4, 5, 6,7,7a-hexahydro-1,3-benzoxazol-3-yl)pyrimidin-2-yl]benzenesulfonamide (hydrochloride salt) (7.5 mg, 20% ). ESI-MS m/z calc. 493.17838, found 494.2 (M+1)⁺; Retention time: 1.6 minutes; LC method A.

### Example 61: Characterization of Compounds 211-226

The compounds in the following tables were prepared in a manner analogous to that described above using commercially available reagents and intermediates described herein.

| | | LCMS Room | | | |
|---|---|---|---|---|---|
| Compound | | Temperature | Calc. | | LCMS |
| number | Structure | (min) | mass | M + 1 | Method |
| 211 | 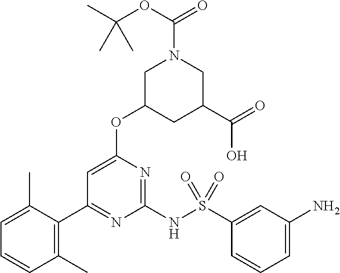 [SMILES: Cc1cccc(C)c1-c1cc(OC2CC(C(=O)O)CN(C(=O)OC(C)(C)C)C2)nc(NS(=O)(=O)c2cccc(N)c2)n1] | 1.51 | 597.226 | 598.2 | A |
| 212 | 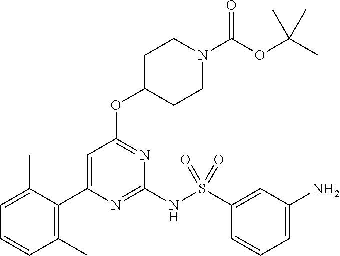 [SMILES: Cc1cccc(C)c1-c1cc(OC2CCN(C(=O)OC(C)(C)C)CC2)nc(NS(=O)(=O)c2cccc(N)c2)n1] | 1.79 | 553.236 | 554.3 | A |
| 213 | 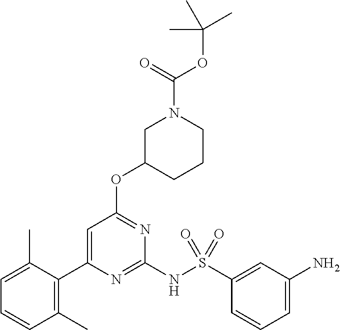 [SMILES: Cc1cccc(C)c1-c1cc(OC2CCCN(C(=O)OC(C)(C)C)C2)nc(NS(=O)(=O)c2cccc(N)c2)n1] | 1.7 | 553.236 | 554.3 | A |
| 214 | 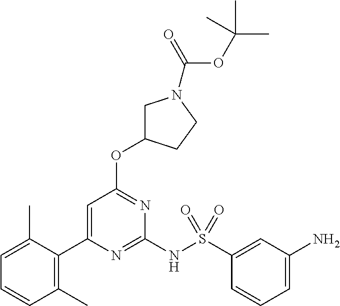 [SMILES: Cc1cccc(C)c1-c1cc(OC2CCN(C(=O)OC(C)(C)C)C2)nc(NS(=O)(=O)c2cccc(N)c2)n1] | 1.68 | 539.22 | 540.3 | A |
| 215 | 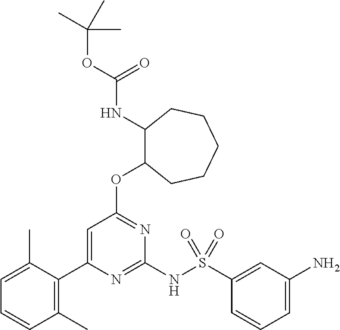 [SMILES: Cc1cccc(C)c1-c1cc(OC2CCCCCC2NC(=O)OC(C)(C)C)nc(NS(=O)(=O)c2cccc(N)c2)n1] | 1.84 | 581.267 | 582.3 | A |
| 216 | 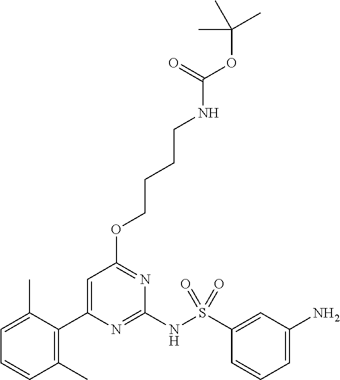 [SMILES: Cc1cccc(C)c1-c1cc(OCCCCNC(=O)OC(C)(C)C)nc(NS(=O)(=O)c2cccc(N)c2)n1] | 1.64 | 541.236 | 542.3 | A |
| 217 | 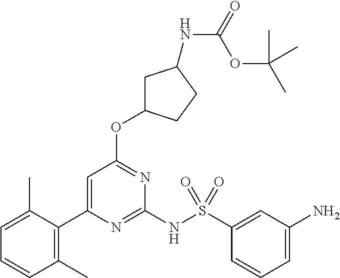 [SMILES: Cc1cccc(C)c1-c1cc(OC2CCC(NC(=O)OC(C)(C)C)C2)nc(NS(=O)(=O)c2cccc(N)c2)n1] | 1.65 | 553.236 | 554.3 | A |
| 218 | 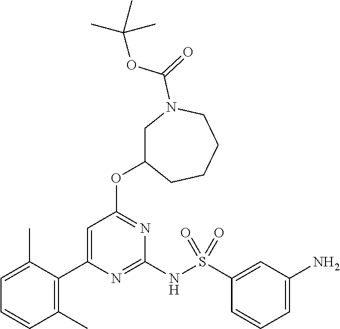 [SMILES: Cc1cccc(C)c1-c1cc(OC2CCCCN(C(=O)OC(C)(C)C)C2)nc(NS(=O)(=O)c2cccc(N)c2)n1] | 1.83 | 567.252 | 568.3 | A |
| 219 | 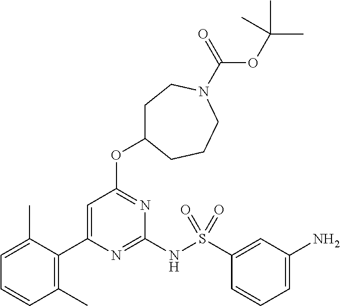 [SMILES: Cc1cccc(C)c1-c1cc(OC2CCCN(C(=O)OC(C)(C)C)CC2)nc(NS(=O)(=O)c2cccc(N)c2)n1] | 1.8 | 567.252 | 568.3 | A |
| 220 | 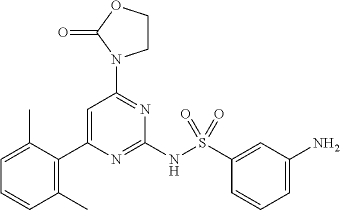 [SMILES: Cc1cccc(C)c1-c1cc(N2CCOC2=O)nc(NS(=O)(=O)c2cccc(N)c2)n1] | 1.25 | 439.131 | 440.1 | A |
| 221 | 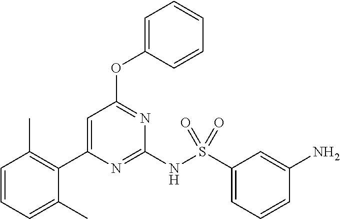 [SMILES: Cc1cccc(C)c1-c1cc(Oc2ccccc2)nc(NS(=O)(=O)c2cccc(N)c2)n1] | 1.68 | 446.141 | 447.3 | A |
| 222 | 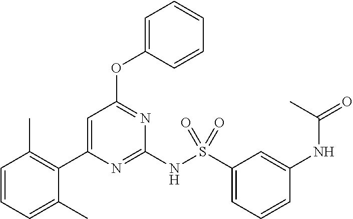 [SMILES: CC(=O)Nc1cccc(S(=O)(=O)Nc2nc(Oc3ccccc3)cc(-c3c(C)cccc3C)n2)c1] | 1.68 | 488.152 | 489.3 | A |
| 223 | 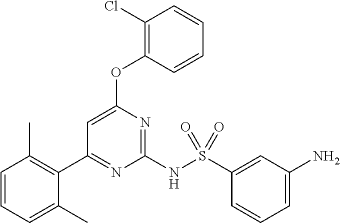 [SMILES: Cc1cccc(C)c1-c1cc(Oc2ccccc2Cl)nc(NS(=O)(=O)c2cccc(N)c2)n1] | 1.74 | 480.102 | 481.2 | A |
| 224 | 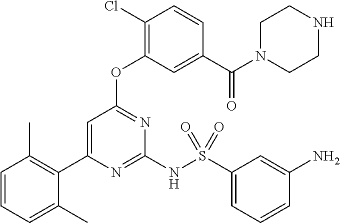 [SMILES: Cc1cccc(C)c1-c1cc(Oc2cc(C(=O)N3CCNCC3)ccc2Cl)nc(NS(=O)(=O)c2cccc(N)c2)n1] | 1.19 | 592.166 | 593.2 | A |
| 225 | 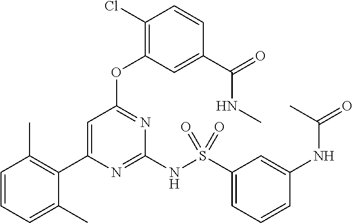 [SMILES: CNC(=O)c1ccc(Cl)c(Oc2cc(-c3c(C)cccc3C)nc(NS(=O)(=O)c3cccc(NC(C)=O)c3)n2)c1] | 1.5 5 | 579.134 | 580.2 | A |
| 226 | 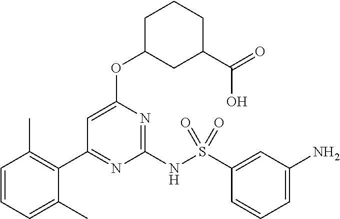 [SMILES: Cc1cccc(C)c1-c1cc(OC2CCCC(C(=O)O)C2)nc(NS(=O)(=O)c2cccc(N)c2)n1] | 1.3 | 496.178 | 497.3 | A |

| Compound | |
|---|---|
| number | NMR |
| 219 | ¹H NMR (400 MHz, dimethylsulfoxide-d₆) δ 13.14-11.27 (bs, 1H), 7.31- |
| | 7.04 (m, 5H), 7.02-6.91 (m, 1H), 6.84-6.60 (m, 1H), 6.47-5.97 (bs, 1H), |
| | 5.79-5.35 (bs, 2H), 5.04-4.85 (bs, 1H), 3.49-3.19 (m, 4H), 2.12-1.93 |
| | (bs, 6H), 1.84-1.54 (m, 5H), 1.41 (s, 9H), 1.28-1.12 (m, 1H) |

### Example 62: Preparation of Compound 227

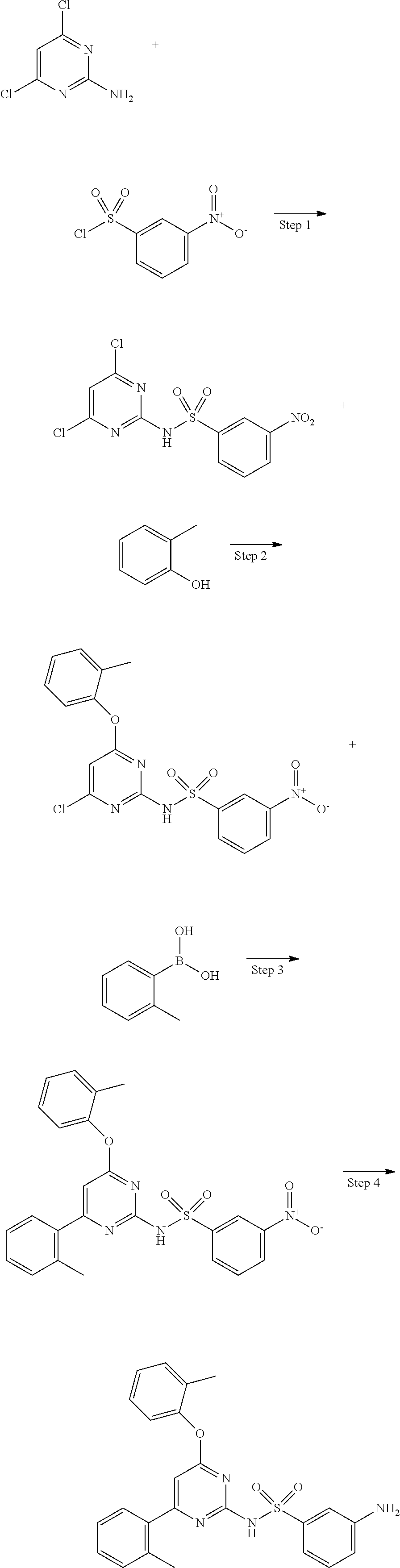

### Step 1: N-(4,6-dichloropyrimidin-2-yl)-3-nitro-benzenesulfonamide

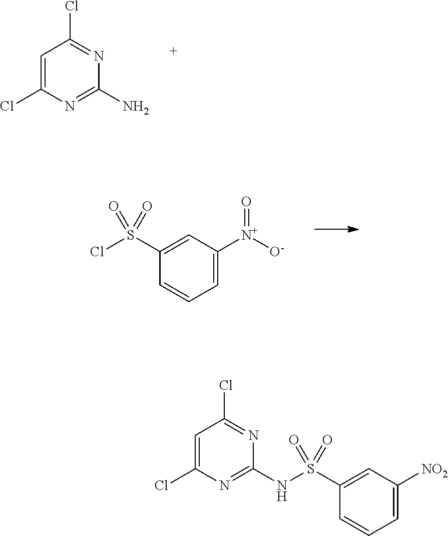 [SMILES: Nc1nc(Cl)cc(Cl)n1 ; O=[N+]([O-])c1cccc(S(=O)(=O)Cl)c1 ; O=[N+]([O-])c1cccc(S(=O)(=O)Nc2nc(Cl)cc(Cl)n2)c1]

To a solution of 4,6-dichloropyrimidin-2-amine (3 g, 20 mmol) in DMF (80 mL) at 0° C. was added sodium hydride (3 g of 60% w/w, 75.01 mmol). The reaction was removed from the cooling bath and allowed to warm to 23° C. over 15 minutes. The reaction was cooled to 0° C. and 3-nitrobenzenesulfonyl chloride (9 g, 40.61 mmol) was added in one portion. The reaction was allowed to warm to 23° C. over 15 minutes and then cooled by to 0° C. before acidifying with acetic acid (20 g, 333.0 mmol). The reaction was diluted with water and ethyl acetate/hexanes (1:1). The organic layer was separated, and the aqueous layer was further extracted with ethyl acetate/hexanes (1:1, 5×). The combined organics were washed with brine, dried over magnesium sulfate, filtered and concentrated in vacuo. The crude residue was separated by flash column chromatography on silica gel (ethyl acetate in hexanes) to afford N-(4,6-dichloropyrimidin-2-yl)-3-nitro-benzenesulfonamide (5.95 g, 79%) as a white solid. ESI-MS m z calc. 347.94867, found 350.49 (M+1)⁺; Retention time: 0.58 minutes; LC method D.

### Step 2: N-[4-chloro-6-(2-methylphenoxy)pyrimidin-2-yl]-3-nitro-benzenesulfonamide

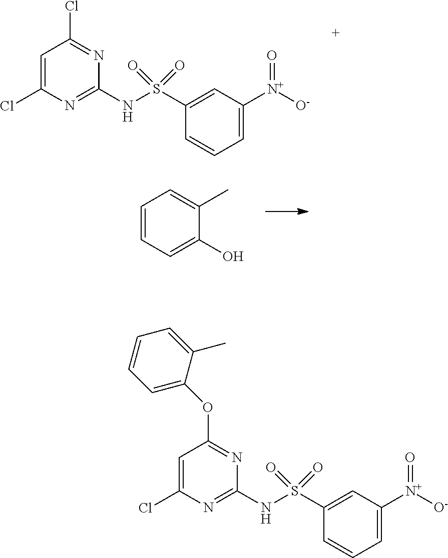

To a mixture of N-(4,6-dichloropyrimidin-2-yl)-3-nitro-benzenesulfonamide (500 mg, 1.432 mmol), o-cresol (approximately 154.9 mg, 280.1 μL, 1.432 mmol) and K₂CO₃ (approximately 593.7 mg, 4.296 mmol) was added DMSO (5 mL) and the mixture was heated at 100° C. for 4 hours. To this reaction mixture was added water and it was then acidified with 2 N HCl. Then precipitate was filtered and purified by reverse phase HPLC using 10-99% acetonitrile in water using HCl as modifier to afford N-[4-chloro-6-(2-methylphenoxy)pyrimidin-2-yl]-3-nitro-benzenesulfonamide (312 mg, 52%). ESI-MS m/z calc. 420.0295, found 421.37 (M+1)⁺; Retention time: 0.73 minutes; LC method A.

### Step 3: N-[4-(2-methylphenoxy)-6-(o-tolyl)pyrimidin-2-yl]-3-nitro-benzenesulfonamide

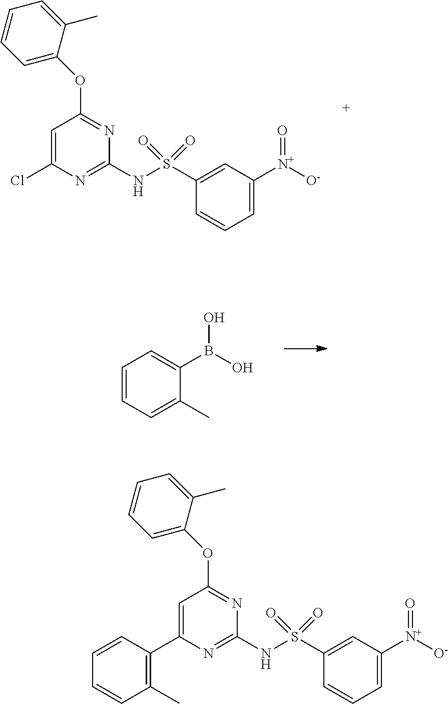

To a solution of N-[4-chloro-6-(2-methylphenoxy)pyrimidin-2-yl]-3-nitro-benzenesulfonamide (100 mg, 0.2376 mmol) and o-tolylboronic acid (approximately 32.30 mg, 0.2376 mmol) in NMP (3 mL) was added potassium carbonate (approximately 365.6 μL of 2 M, 0.7311 mmol) followed by Pd(dppf)C₁₂ (approximately 149.3 mg, 0.1828 mmol). The reaction mixture was flushed with nitrogen. It was heated in a sealed tube at 100° C. for 1 hour. It was filtered and was purified by reverse phase HPLC using 10-99% acetonitrile in water to afford N-[4-(2-methylphenoxy)-6-(o-tolyl)pyrimidin-2-yl]-3-nitro-benzenesulfonamide (71 mg, 82%) ESI-MS m/z calc. 476.11545, found 477.5 (M+1)⁺; Retention time: 0.75 minutes; LC method D.

### Step 4: 3-Amino-N-[4-(2-methylphenoxy)-6-(o-tolyl)pyrimidin-2-yl]benzenesulfonamide (Compound 227)

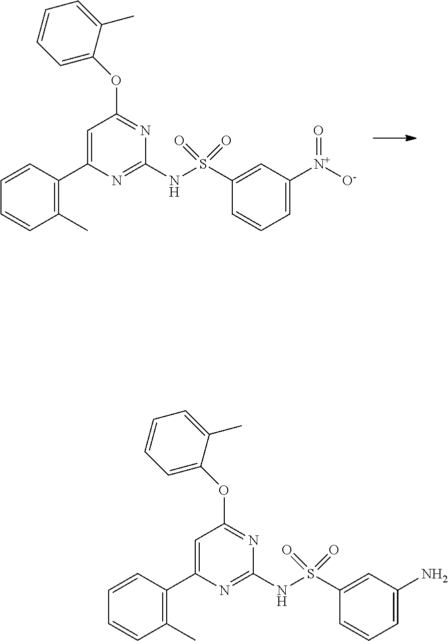 [SMILES: Cc1ccccc1Oc1cc(-c2ccccc2C)nc(NS(=O)(=O)c2cccc([N+](=O)[O-])c2)n1 ; Cc1ccccc1Oc1cc(-c2ccccc2C)nc(NS(=O)(=O)c2cccc(N)c2)n1]

To a solution of N-[4-(2-methylphenoxy)-6-(o-tolyl)pyrimidin-2-yl]-3-nitro-benzenesulfonamide (25 mg, 0.05247 mmol) in MeOH (1 mL) and DMF (250 μL) was added Pd (12 mg of 10% w/w, 0.01128 mmol) and stirred under H₂ for 90 minutes. The reaction mixture was filtered and was purified by reverse phase HPLC using 1-99% acetonitrile in water using HCl as a modifier to afford 3-amino-N-[4-(2-methylphenoxy)-6-(o-tolyl)pyrimidin-2-yl]benzenesulfonamide (6 mg, 26%) ESI-MS m/z calc. 446.14127, found 447.49 (M+1)⁺; Retention time: 1.76 minutes; LC method A.

### Example 63: Preparation of Compound 228)

### Step 1: 3,3-Bis(methylsulfanyl)-1-(o-tolyl)prop-2-en-1-one

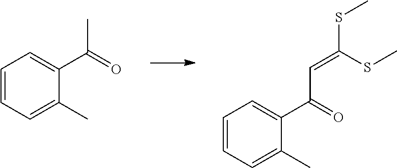

A 1 L round-bottomed flask equipped with a magnetic stir bar was dried with a heat gun under vacuum and purged with nitrogen; to this was added 1-(o-tolyl)ethanone (21.128 g, 157.5 mmol). Dry tetrahydrofuran (500 mL) was added and this solution was cooled to 0° C. 60% NaH (16.101 g, 402.6 mmol) was added in three portions under a blanket of nitrogen, and the reaction mixture was warmed to room temperature over 45 minutes. The mixture was cooled to 0° C., upon which carbon disulfide (14.0 mL, 232.8 mmol) was added. The solution was then warmed to room temperature over 45 minutes. The reaction mixture was cooled to 0° C., upon which iodomethane (22.0 mL, 353.4 mmol) was added. The mixture was stirred at 0° C. for 30 minutes, then warmed to room temperature over 20 hours, maintaining a water bath around the flask (CAUTION: hydrogen gas evolution and slight exotherm). The reaction was quenched by a slow transfer onto ice-cold 1 N hydrochloric acid (500 mL). The mixture was extracted with ethyl acetate (3×300 mL). The combined organic extracts was washed with water (300 mL) and saturated aqueous sodium chloride solution (200 mL), then dried over sodium sulfate, filtered, and evaporated in vacuo to give a brown solid, 3,3-bis(methylsulfanyl)-1-(o-tolyl)prop-2-en-1-one (37.0 g, 99%). ESI-MS m/z calc. 238.0486, found 239.0 (M+1)⁺; Retention time: 0.61 minutes; LC method D.

### Step 2: 4-Methylsulfanyl-6-(o-tolyl)pyrimidin-2-amine

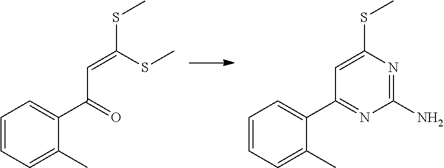 [SMILES: CSC(=CC(=O)c1ccccc1C)SC ; CSc1cc(-c2ccccc2C)nc(N)n1]

To a 1 L round-bottomed flask equipped with a magnetic stir bar were added 3,3-bis(methylsulfanyl)-1-(o-tolyl)prop-2-en-1-one (37.54 g, 157.5 mmol), dimethylformamide (350 mL), guanidine carbonate (59.56 g, 330.6 mmol) and potassium carbonate (80.23 g, 580.5 mmol), in this order. This slurry was heated at 110° C. for 16 hours then at 100° C. for 20 hours. After cooling to room temperature, the flask was opened (CAUTION: stench!) and the contents were quenched by transferring onto cold water (500 mL). This mixture was extracted with ethyl acetate (3×500 mL), then the organic layers were combined and washed with water (2×500 mL) and saturated aqueous sodium chloride solution (500 mL), dried over sodium sulfate, filtered, and evaporated in vacuo. This crude product was purified by silica gel chromatography (330 g of silica, 0 to 30% gradient of ethyl acetate/hexanes) to give a beige solid, 4-methylsulfanyl-6-(o-tolyl)pyrimidin-2-amine (20.43 g, 56%) ESI-MS m/z calc. 231.08302, found 232.0 (M+1)⁺; Retention time: 0.93 minutes; LC method A.

### Step 3: N-[4-Methylsulfanyl-6-(o-tolyl)pyrimidin-2-yl]-3-nitro-benzenesulfonamide

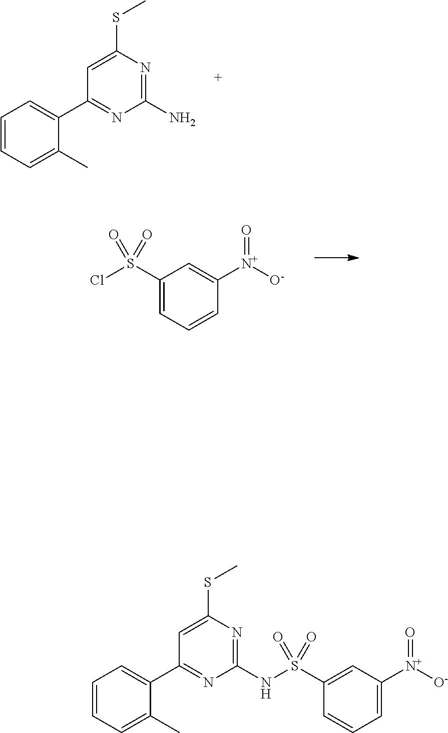 [SMILES: CSc1cc(-c2ccccc2C)nc(N)n1 ; O=[N+]([O-])c1cccc(S(=O)(=O)Cl)c1 ; CSc1cc(-c2ccccc2C)nc(NS(=O)(=O)c2cccc([N+](=O)[O-])c2)n1]

A 250 mL round-bottomed flask equipped with a magnetic stir bar was dried with a heat gun under vacuum and purged with nitrogen; 4-methylsulfanyl-6-(o-tolyl)pyrimidin-2-amine (7.61 g, 32.90 mmol) and dimethylformamide (80 mL) were added, and this solution was cooled to 0° C. 60% NaH (3.20 g, 80.01 mmol) was added in one portion, and the reaction mixture was warmed to room temperature over 15 minutes. The mixture was cooled to 0° C., upon which 3-nitrobenzenesulfonyl chloride (9.31 g, 42.01 mmol) was added in three portions (CAUTION: hydrogen gas evolution). This solution was stirred at room temperature for 80 minutes, then quenched by a slow transfer onto cold 1 N HCl (100 mL). The mixture was extracted with ethyl acetate (3×100 mL). The combined organic extracts were washed with water (2×150 mL) and saturated aqueous sodium chloride solution (150 mL), then dried over sodium sulfate, filtered, and evaporated in vacuo. This crude product was purified by silica gel chromatography (120 g of silica, 0 to 30% gradient of ethyl acetate/hexanes) to give N-[4-methylsulfanyl-6-(o-tolyl)pyrimidin-2-yl]-3-nitro-benzenesulfonamide (2.582 g, 12%) ESI-MS m/z calc. 416.0613, found 417.1 (M+1)⁺; Retention time: 0.64 minutes; LC method D.

### Step 4: N-[4-Methylsulfonyl-6-(o-tolyl)pyrimidin-2-yl]-3-nitro-benzenesulfonamide

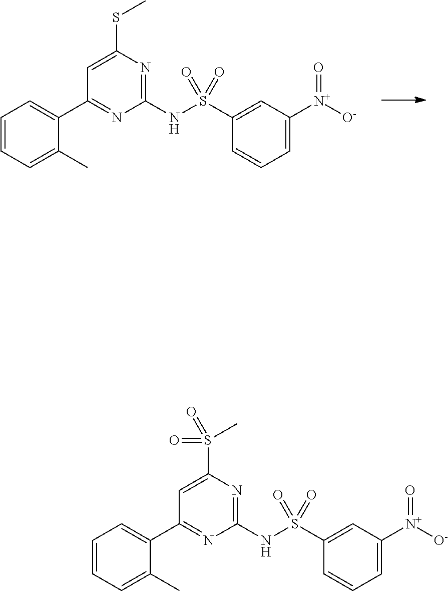 [SMILES: CSc1cc(-c2ccccc2C)nc(NS(=O)(=O)c2cccc([N+](=O)[O-])c2)n1 ; Cc1ccccc1-c1cc(S(C)(=O)=O)nc(NS(=O)(=O)c2cccc([N+](=O)[O-])c2)n1]

To a 100 mL round-bottomed flask equipped with a magnetic stir bar, N-[4-methylsulfanyl-6-(o-tolyl)pyrimidin-2-yl]-3-nitro-benzenesulfonamide (2.582 g, 4.030 mmol) and dichloromethane (40 mL) were added, followed by 77% m-CPBA (2.151 g, 9.598 mmol). This mixture was stirred at room temperature for 90 minutes, upon which a second addition of 77% m-CPBA (1.035 g, 4.618 mmol) was made. After 1 hour of stirring at room temperature, the reaction mixture was quenched with solid sodium thiosulfate (3.205 g, 20.27 mmol). This mixture was stirred for another 90 minutes at room temperature. The reaction mixture was diluted with dichloromethane (100 mL), then washed with water (100 mL), dried over sodium sulfate, filtered, and evaporated in vacuo. This solid was then partially dissolved in dichloromethane (15 mL) and filtered in vacuo on a Buchner funnel to remove the m-chlorobenzoic acid waste. The remaining solution was then purified by silica gel chromatography (40 g of silica, 0 to 60% gradient of ethyl acetate/hexanes) to give 3 batches of product, which were N-[4-methylsulfonyl-6-(o-tolyl)pyrimidin-2-yl]-3-nitro-benzenesulfonamide (1.7153 g, 79%). ¹H NMR (400 MHz, chloroform-d) δ 8.97 (t, J=2.0 Hz, 1H), 8.48-8.41 (m, 2H), 7.75 (s, 1H), 7.71 (t, J=8.1 Hz, 1H), 7.47-7.36 (m, 2H), 7.35-7.28 (m, 2H), 3.27 (s, 3H), 2.44 (s, 3H). ESI-MS m/z calc. 448.05112, found 449.1 (M+1)⁺; Retention time: 0.6 minutes; LC method D.

### Step 5-6: 3-Amino-N-[4-(3-chlorophenoxy)-6-(o-tolyl)pyrimidin-2-yl]benzenesulfonamide (Compound 228)

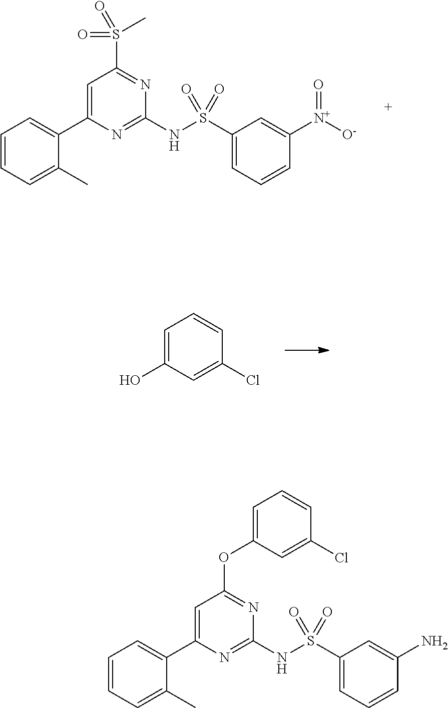 [SMILES: Cc1ccccc1-c1cc(S(C)(=O)=O)nc(NS(=O)(=O)c2cccc([N+](=O)[O-])c2)n1 ; Oc1cccc(Cl)c1 ; Cc1ccccc1-c1cc(Oc2cccc(Cl)c2)nc(NS(=O)(=O)c2cccc(N)c2)n1]

To a 20 mL vial equipped with a magnetic stir bar, N-[4-methylsulfonyl-6-(o-tolyl)pyrimidin-2-yl]-3-nitro-benzenesulfonamide (171.1 mg, 0.3815 mmol), N-methylpyrrolidinone (5.0 mL) and 3-chlorophenol (148.6 mg, 1.156 mmol) were added, followed by potassium carbonate (242.3 mg, 1.753 mmol). This solution was stirred at 100° C. for 22 hours. The reaction mixture was then cooled to room temperature, quenched with 1 N HCl (4 mL), and extracted with ethyl acetate (3×4 mL). The combined organic extracts were washed with water (2×4 mL) and saturated aqueous sodium chloride solution (3 mL), then dried over sodium sulfate, filtered, and evaporated in vacuo. The crude product obtained above was dissolved in ethanol (2.2 mL) and transferred to a 10 mL vial equipped with a magnetic stir bar. Aqueous HCl (0.8 mL of 0.5 M, 0.4000 mmol) was added, followed by a fine dust of iron (145.3 mg, 2.602 mmol). This reaction mixture was stirred at 70° C. for 10 minutes. It was cooled to room temperature, filtered, and purified by reverse phase HPLC (1-99% acetonitrile in water using HCl as a modifier) to give 3-amino-N-[4-(3-chlorophenoxy)-6-(o-tolyl)pyrimidin-2-yl]benzenesulfonamide (hydrochloride salt) (118.2 mg, 62%). ESI-MS m/z calc. 466.08664, found 467.1 (M+1)⁺; Retention time: 1.79 minutes; LC method A.

### Example 64: Preparation of Compound 229

### Step 1: N-[4-[4-(Hydroxymethyl)phenoxy]-6-(o-tolyl)pyrimidin-2-yl]-3-nitro-benzenesulfonamide

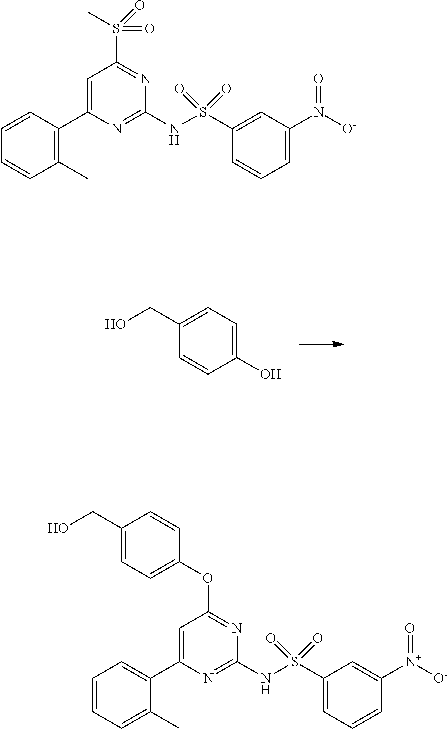 [SMILES: Cc1ccccc1-c1cc(S(C)(=O)=O)nc(NS(=O)(=O)c2cccc([N+](=O)[O-])c2)n1 ; OCc1ccc(O)cc1 ; Cc1ccccc1-c1cc(Oc2ccc(CO)cc2)nc(NS(=O)(=O)c2cccc([N+](=O)[O-])c2)n1]

To a mixture of N-[4-methylsulfonyl-6-(o-tolyl)pyrimidin-2-yl]-3-nitro-benzenesulfonamide (50 mg, 0.1115 mmol), 4-(hydroxymethyl)phenol (approximately 27.68 mg, 0.2230 mmol), and K₂CO₃ (approximately 61.64 mg, 0.4460 mmol), was added NMP (2 mL). The reaction mixture was heated at 100° C. overnight, then was filtered and was purified by reverse phase HPLC using 10-99% acetonitrile in water to afford N-[4-[4-(hydroxymethyl)phenoxy]-6-(o-tolyl)pyrimidin-2-yl]-3-nitro-benzenesulfonamide (20.0 mg, 36%). ESI-MS m/z calc. 492.11035, found 493.5 (M+1)⁺; Retention time: 0.62 minutes; LC method D.

### Step 2: 3-Amino-N-[4-[4-(hydroxymethyl)phenoxy]-6-(o-tolyl)pyrimidin-2-(Compound 229)

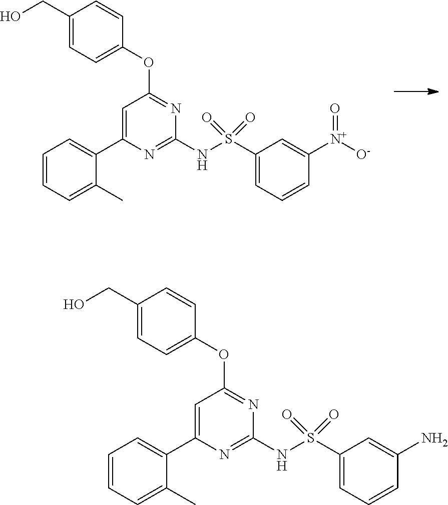 [SMILES: Cc1ccccc1-c1cc(Oc2ccc(CO)cc2)nc(NS(=O)(=O)c2cccc([N+](=O)[O-])c2)n1 ; Cc1ccccc1-c1cc(Oc2ccc(CO)cc2)nc(NS(=O)(=O)c2cccc(N)c2)n1]

To a solution of N-[4-[4-(hydroxymethyl)phenoxy]-6-(o-tolyl)pyrimidin-2-yl]-3-nitro-benzenesulfonamide (20 mg, 0.04061 mmol) in a mixture of MeOH (1 mL) and DMF (200 μL) was added palladium on carbon (approximately 21.60 mg of 10% w/w, 0.02030 mmol) and it was flushed with H₂. The mixture was stirred under H₂ for 1 hour. It was filtered and was purified by reverse phase HPLC using 1-99% acetonitrile in water using HCl as modifier to afford 3-amino-N-[4-[4-(hydroxymethyl)phenoxy]-6-(o-tolyl)pyrimidin-2-yl]benzenesulfonamide (3.20 mg, 17%) ESI-MS m/z calc. 462.13617, found 463.49 (M+1)⁺; Retention time: 1.39 minutes; LC method F.

### Example 65: Preparation of Compound 230

### Step 1: N-(4-chloro-6-phenoxy-pyrimidin-2-yl)-3-nitro-benzenesulfonamide

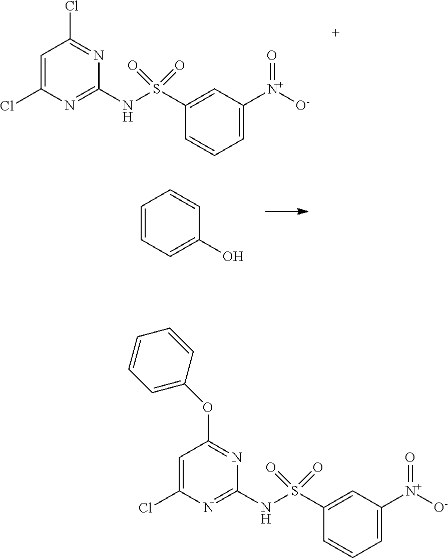 [SMILES: O=[N+]([O-])c1cccc(S(=O)(=O)Nc2nc(Cl)cc(Cl)n2)c1 ; Oc1ccccc1 ; O=[N+]([O-])c1cccc(S(=O)(=O)Nc2nc(Cl)cc(Oc3ccccc3)n2)c1]

To a mixture of N-(4,6-dichloropyrimidin-2-yl)-3-nitro-benzenesulfonamide (500 mg, 1.432 mmol), phenol (approximately 134.8 mg, 127.2 μL, 1.432 mmol), and K₂CO₃ (approximately 593.7 mg, 4.296 mmol) was added DMSO (5 mL) and the mixture was heated at 100° C. for 4 hours. To this reaction mixture was added water and then it was acidified with 2 N HCl. The solid was filtered and the precipitate was purified by reverse phase HPLC using 10-99 % acetonitrile in water using HCl as modifier to afford N-(4-chloro-6-phenoxy-pyrimidin-2-yl)-3-nitro-benzenesulfonamide (340 mg, 58%) ESI-MS m/z calc. 406.01385, found 407.36 (M+1)⁺; Retention time: 0.69 minutes; LC method A.

### Step 2: 3-nitro-N-[4-(o-tolyl)-6-phenoxy-pyrimidin-2-yl]benzenesulfonamide

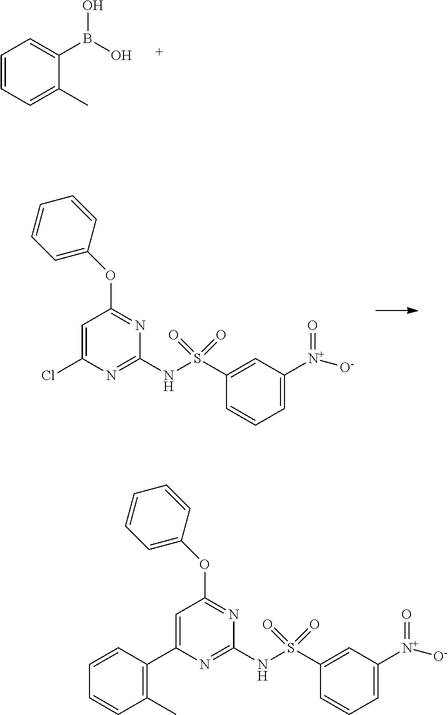

To a solution of N-(4-chloro-6-phenoxy-pyrimidin-2-yl)-3-nitro-benzenesulfonamide (100 mg, 0.2458 mmol), o-tolylboronic acid (approximately 43.44 mg, 0.3195 mmol) in NMP (3 mL) was added Pd(dppf)C₁₂-DCM (approximately 200.7 mg, 0.2458 mmol) and potassium carbonate (approximately 491.6 μL of 2 M, 0.9832 mmol). The reaction mixture was flushed with nitrogen. It was heated at 100° C. for 60 minutes. It was filtered, and was purified by reverse phase HPLC using 10-99% acetonitrile in water to afford 3-nitro-N-[4-(o-tolyl)-6-phenoxy-pyrimidin-2-yl]benzenesulfonamide (67 mg, 59%) ESI-MS m/z calc. 462.0998, found 463.45 (M+1)⁺; Retention time: 0.73 minutes; LC method D.

### Step 3: 3-amino-N-[4-(o-tolyl)-6-phenoxy-pyrimidin-2-yl]benzenesulfonamide (Compound 230)

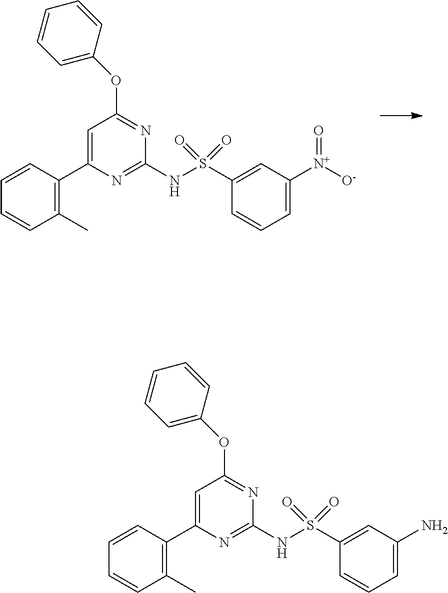 [SMILES: Cc1ccccc1-c1cc(Oc2ccccc2)nc(NS(=O)(=O)c2cccc([N+](=O)[O-])c2)n1 ; Cc1ccccc1-c1cc(Oc2ccccc2)nc(NS(=O)(=O)c2cccc(N)c2)n1]

To a solution of 3-nitro-N-[4-(o-tolyl)-6-phenoxy-pyrimidin-2-yl]benzenesulfonamide (10 mg, 0.02162 mmol) in MeOH (1 mL) was added palladium on carbon (approximately 11.50 mg of 10% w/w, 0.01081 mmol) and was stirred under H₂ atmosphere for 90 minutes. It was filtered and was purified by reverse phase HPLC using 1-99% acetonitrile in water using HCl as modifier to afford 3-amino-N-[4-(o-tolyl)-6-phenoxy-pyrimidin-2-yl]benzenesulfonamide (5.7 mg, 61%) ESI-MS m/z calc. 432.1256, found 433.48 (M+1)⁺; Retention time: 1.69 minutes; LC method A.

### Example 66: Preparation of Compound 231

### Step 1: N-[3-[[4-(o-tolyl)-6-phenoxy-pyrimidin-2-yl]sulfamoyl]phenyl]acetamide (Compound 231)

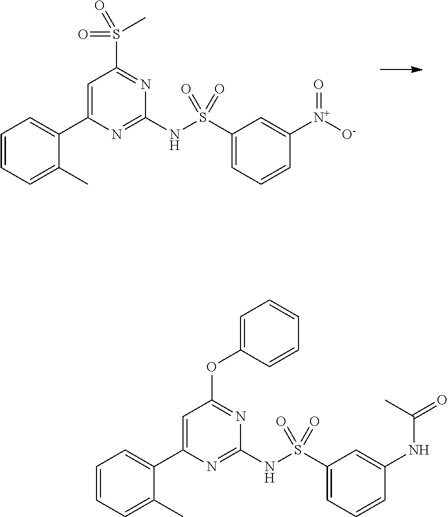 [SMILES: Cc1ccccc1-c1cc(S(C)(=O)=O)nc(NS(=O)(=O)c2cccc([N+](=O)[O-])c2)n1 ; CC(=O)Nc1cccc(S(=O)(=O)Nc2nc(Oc3ccccc3)cc(-c3ccccc3C)n2)c1]

Stage 1: To a 10 mL vial equipped with a magnetic stir bar, N-[4-methylsulfonyl-6-(o-tolyl)pyrimidin-2-yl]-3-nitro-benzenesulfonamide (60.0 mg, 0.1338 mmol), N-methylpyrrolidinone (2.0 mL) and phenol (50.0 mg, 0.5313 mmol) were added, followed by potassium carbonate (75.0 mg, 0.5427 mmol). This mixture was stirred at 110° C. for 19 hours. The reaction mixture was then cooled to room temperature, quenched with 1 N HCl (3 mL), and extracted with ethyl acetate (3×3 mL). The combined organic extracts were washed with water (2×5 mL) and saturated aqueous sodium chloride solution (5 mL), then dried over sodium sulfate, filtered, and evaporated in vacuo.

Stage 2: The crude product from the first stage was dissolved in ethanol (2.0 mL) and transferred to a 10 mL vial equipped with a magnetic stir bar. This solution was purged with a balloon of hydrogen gas for 5 minutes. The cap was briefly removed, and 20% Pd(OH)₂/C (6.0 mg, 0.008545 mmol) was added. This reaction mixture was stirred under hydrogen gas (2 L, 79.37 mmol) at 60° C. for 19 hours. It was then filtered through Celite and rinsed with ethanol (3 mL), then evaporated in vacuo.

Stage 3: The crude product from the second stage was dissolved in dimethylformamide (0.7 mL) and transferred to a 3 mL vial equipped with a magnetic stir bar. To this solution, triethylamine (20 μL, 0.1435 mmol), acetic anhydride (20 μL, 0.2120 mmol) and 4-(dimethylamino)pyridine (1.0 mg, 0.008185 mmol) were added. After stirring at room temperature for 1 hour, the reaction mixture was filtered and purified by reverse phase HPLC (1-99% acetonitrile in water using HCl as a modifier) to give N-[3-[[4-(o-tolyl)-6-phenoxy-pyrimidin-2-yl]sulfamoyl]phenyl]acetamide (39.2 mg, 62%) ESI-MS m/z calc. 474.13617, found 475.2 (M+1)⁺; Retention time: 1.63 minutes; LC method A.

### Example 67: Characterization of Compounds 232-241

The compounds in the following tables were prepared in a manner analogous to that described above using commercially available reagents and intermediates described herein.

| | | LCMS | | | |
|---|---|---|---|---|---|
| | | Room | | | |
| Compound | | Temperature | Calc. | | LCMS |
| number | Structure | (min) | mass | M + 1 | Method |
| 232 | 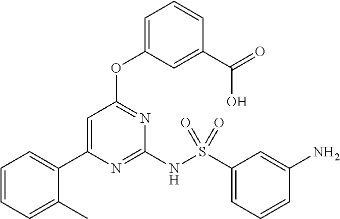 [SMILES: Cc1ccccc1-c1cc(Oc2cccc(C(=O)O)c2)nc(NS(=O)(=O)c2cccc(N)c2)n1] | 1.39 | 476.115 | 477.1 | A |
| 233 | 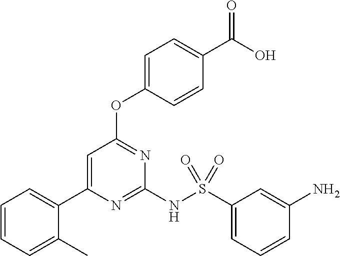 [SMILES: Cc1ccccc1-c1cc(Oc2ccc(C(=O)O)cc2)nc(NS(=O)(=O)c2cccc(N)c2)n1] | 1.41 | 476.115 | 477.1 | A |
| 234 | 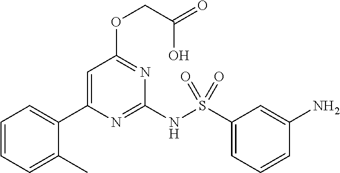 [SMILES: Cc1ccccc1-c1cc(OCC(=O)O)nc(NS(=O)(=O)c2cccc(N)c2)n1] | 0.97 | 414.1 | 415.1 | A |
| 235 | 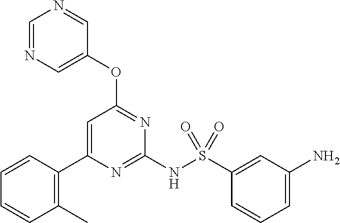 [SMILES: Cc1ccccc1-c1cc(Oc2cncnc2)nc(NS(=O)(=O)c2cccc(N)c2)n1] | 1.26 | 434.116 | 435.48 | A |
| 236 | 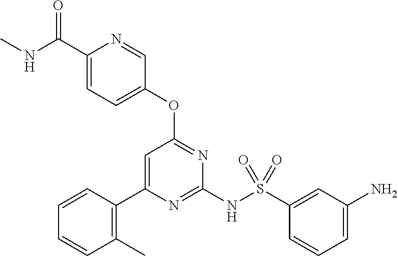 [SMILES: CNC(=O)c1ccc(Oc2cc(-c3ccccc3C)nc(NS(=O)(=O)c3cccc(N)c3)n2)cn1] | 1.38 | 490.142 | 491.47 | A |
| 237 | 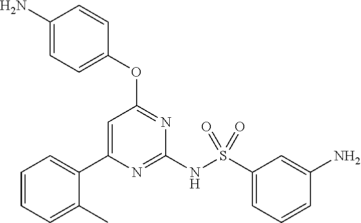 [SMILES: Cc1ccccc1-c1cc(Oc2ccc(N)cc2)nc(NS(=O)(=O)c2cccc(N)c2)n1] | 1.08 | 447.137 | 448.47 | A |
| 238 | 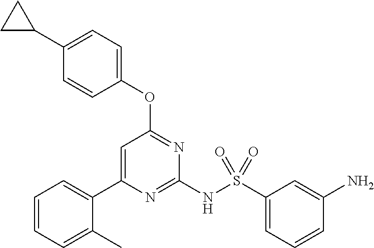 [SMILES: Cc1ccccc1-c1cc(Oc2ccc(C3CC3)cc2)nc(NS(=O)(=O)c2cccc(N)c2)n1] | 1.92 | 472.157 | 473.5 | A |
| 239 | 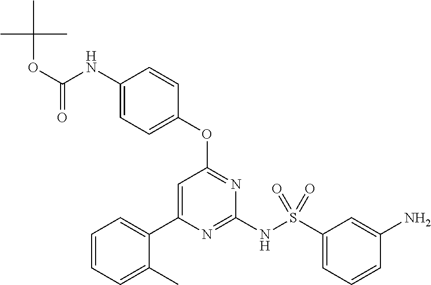 [SMILES: Cc1ccccc1-c1cc(Oc2ccc(NC(=O)OC(C)(C)C)cc2)nc(NS(=O)(=O)c2cccc(N)c2)n1] | 1.85 | 547.189 | 548.51 | A |
| 240 | 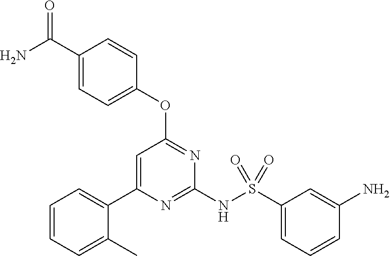 [SMILES: Cc1ccccc1-c1cc(Oc2ccc(C(N)=O)cc2)nc(NS(=O)(=O)c2cccc(N)c2)n1] | 1.29 | 475.131 | 476.48 | A |
| 241 | 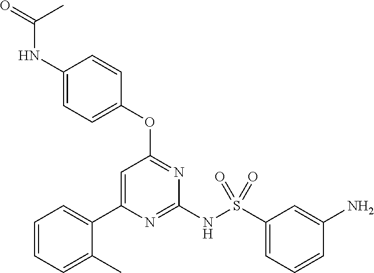 [SMILES: CC(=O)Nc1ccc(Oc2cc(-c3ccccc3C)nc(NS(=O)(=O)c3cccc(N)c3)n2)cc1] | 1.38 | 489.147 | 490.49 | A |

### Example 68: Preparation of Compound 242, Compound 243, and Compound 244

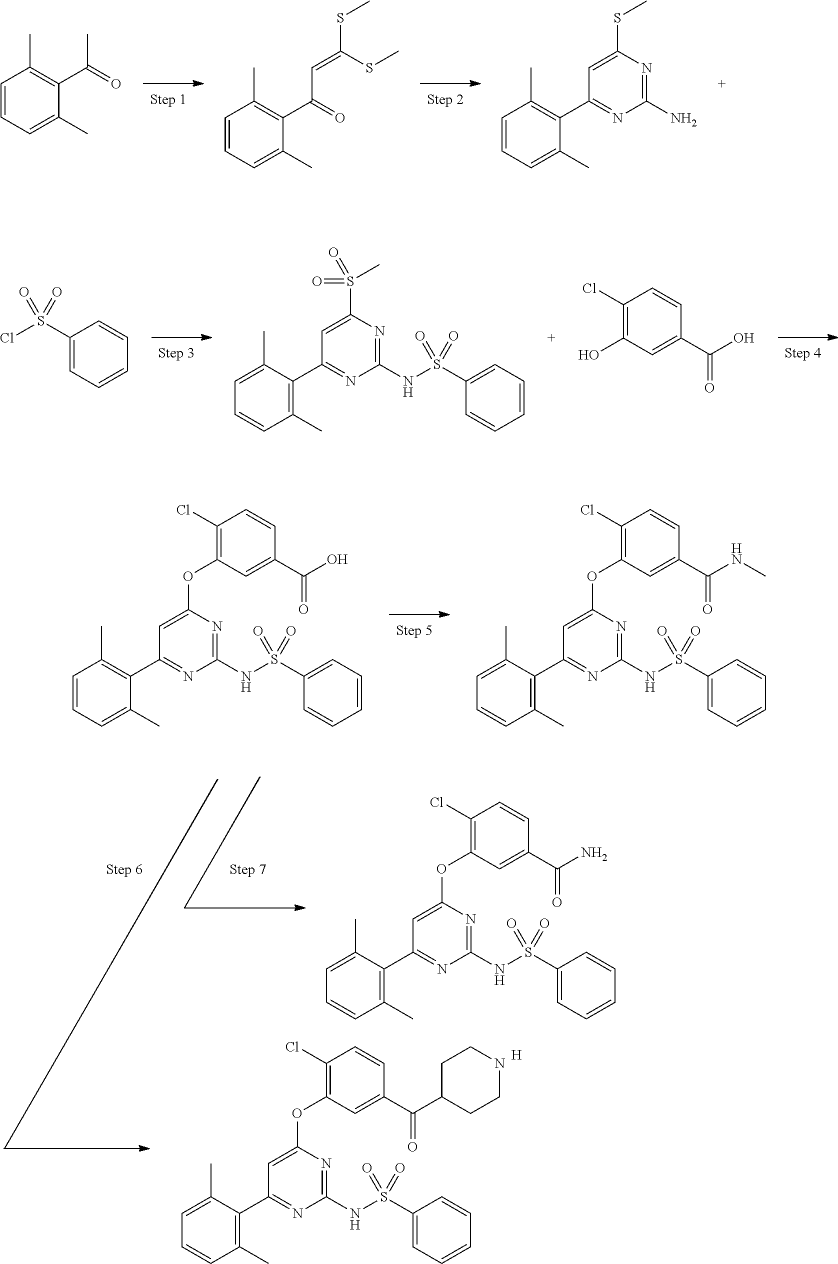

### Step 1: 1-(2,6-Dimethylphenyl)-3,3-bis(methylsulfanyl)prop-2-en-1-one

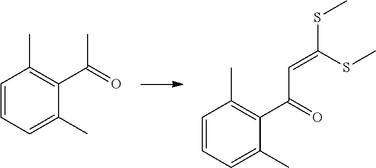 [SMILES: CC(=O)c1c(C)cccc1C ; CSC(=CC(=O)c1c(C)cccc1C)SC]

A 1 L round-bottomed flask equipped with a magnetic stir bar was dried with a heat gun under vacuum and purged with nitrogen; to this was added 1-(2,6-dimethylphenyl)ethanone (20.07 g, 135.4 mmol). Dry tetrahydrofuran (500 mL) was added and this solution was cooled to 0° C. 95% NaH (7.50 g, 296.9 mmol) was added in three portions under a blanket of nitrogen, and the reaction mixture was warmed to room temperature over 45 minutes. The mixture was cooled to 0° C., upon which carbon disulfide (12.0 mL, 199.5 mmol) was added. The solution was then warmed to room temperature over 45 minutes. The reaction mixture was cooled to 0° C., upon which iodomethane (20.0 mL, 321.3 mmol) was added. The mixture was stirred at 0° C. for 30 minutes, then warmed to room temperature over 3 hours, cooling the flask when necessary (CAUTION: hydrogen gas evolution). The reaction was quenched by a slow transfer onto ice-cold water (500 mL). The mixture was extracted with ethyl acetate (3×300 mL). The combined organic extracts were washed with water (300 mL) and saturated aqueous sodium chloride solution (200 mL), then dried over sodium sulfate, filtered, and evaporated in vacuo. This oil was purified by a short pad of silica gel (150 g of silica, elution with 2 L of 1:1 ethyl acetate/hexanes) to give a brown solid, 1-(2,6-dimethylphenyl)-3,3-bis(methylsulfanyl)prop-2-en-i-one (34.0 g, 100%) ESI-MS m/z calc. 252.06425, found 253.0 (M+1)⁺; Retention time: 0.63 minutes; LC method D.

### Step 2: 4-(2,6-Dimethylphenyl)-6-methylsulfanyl-pyrimidin-2-amine

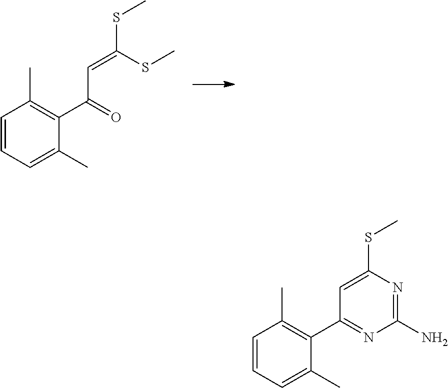 [SMILES: CSC(=CC(=O)c1c(C)cccc1C)SC ; CSc1cc(-c2c(C)cccc2C)nc(N)n1]

To a 1 L round-bottomed flask equipped with a magnetic stir bar were added 1-(2,6-dimethylphenyl)-3,3-bis(methylsulfanyl)prop-2-en-1-one (34.0 g, 134.7 mmol), dimethylformamide (350 mL), guanidine carbonate (50.0 g, 277.5 mmol) and potassium carbonate (70.0 g, 506.5 mmol), in this order. This slurry was heated at 105° C. for 19 hours. After cooling to room temperature, the flask was opened (CAUTION: stench!) and the contents were quenched by transferring onto cold water (500 mL). The product precipitated out of solution, and this solid was collected on a Buchner funnel and dried under vacuum: 4-(2,6-dimethylphenyl)-6-methylsulfanyl-pyrimidin-2-amine (20.117 g, 49%). ¹H NMR (400 MHz, dimethylsulfoxide-d₆) δ 7.16 (dd, J=8.3, 6.7 Hz, 1H), 7.07 (d, J=7.6 Hz, 2H), 6.68 (s, 2H), 6.38 (s, 1H), 2.46 (s, 3H), 2.05 (s, 6H). ESI-MS m/z calc. 245.09866, found 246.0 (M+1)⁺; Retention time: 0.4 minutes; LC method D.

### Step 3-4: N-[4-(2,6-dimethylphenyl)-6-methylsulfonyl-pyrimidin-2-yl]benzenesulfonamide

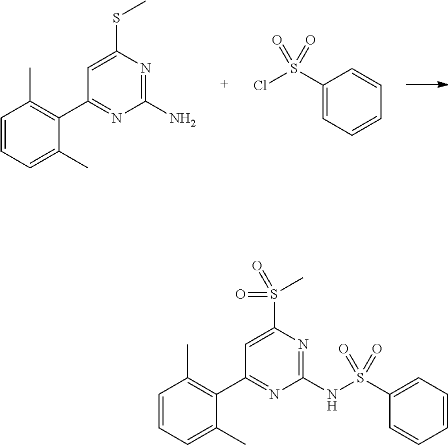 [SMILES: CSc1cc(-c2c(C)cccc2C)nc(N)n1 ; O=S(=O)(Cl)c1ccccc1 ; Cc1cccc(C)c1-c1cc(S(C)(=O)=O)nc(NS(=O)(=O)c2ccccc2)n1]

To a 20 mL vial equipped with a magnetic stir bar, 4-(2,6-dimethylphenyl)-6-methylsulfanyl-pyrimidin-2-amine (0.6536 g, 2.398 mmol) and dimethylformamide (8.0 mL) were added, and this solution was cooled to 0° C. 60% NaH (0.296 g, 7.401 mmol) was added in one portion, and the reaction mixture was warmed to room temperature over 5 minutes. The mixture was cooled to 0° C., upon which benzenesulfonyl chloride (0.50 mL, 3.918 mmol) was added in one portion. This solution was stirred in a room-temperature water bath for 10 min (CAUTION: hydrogen gas evolution), then quenched with cold 1 N HCl (15 mL). The mixture was extracted with ethyl acetate (3×20 mL). The combined organic extracts were washed with water (2×30 mL) and saturated aqueous sodium chloride solution (30 mL), then dried over sodium sulfate, filtered, and evaporated in vacuo. This crude product was purified by silica gel chromatography (24 g of silica, 0 to 30% gradient of ethyl acetate/hexanes) to give 403.7 mg of an approximatively 80% pure intermediate.

To a 20 mL vial equipped with a magnetic stir bar, the product from the previous stage (0.4037 g, -0.8 mmol) and dichloromethane (10.0 mL) were added, followed by 77% m-CPBA (756.1 mg, 3.374 mmol). This mixture was stirred at room temperature for 50 minutes, upon which it was quenched with sodium thiosulfate (902.5 mg, 5.708 mmol). This mixture was stirred for another 50 minutes at room temperature. The reaction mixture was diluted with dichloromethane (20 mL), then washed with water (20 mL), dried over sodium sulfate, filtered, and evaporated in vacuo. This solid was then partially dissolved in dichloromethane (4 mL) and filtered in vacuo on a Buchner funnel to remove the m-chlorobenzoic acid waste. The remaining solution was then purified by silica gel chromatography (24 g of silica, 0 to 50% gradient of ethyl acetate/hexanes) to give 2 products, the desired N-[4-(2,6-dimethylphenyl)-6-methylsulfonyl-pyrimidin-2-yl]benzenesulfonamide (0.2615 g, 26%) ESI-MS m/z calc. 417.0817, found 418.2 (M+1)⁺; Retention time: 1.56 minutes; LC method A.

### Step 4: 3-[2-(Benzenesulfonamido)-6-(2,6-dimethylphenyl)pyrimidin-4-yl]oxy-4-chloro-benzoic acid

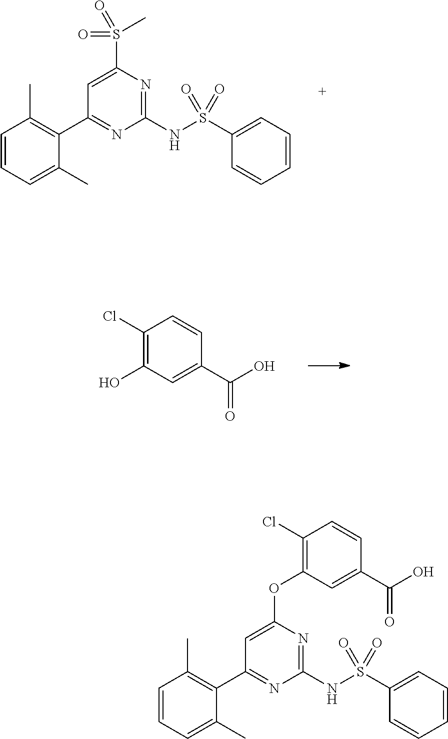 [SMILES: Cc1cccc(C)c1-c1cc(S(C)(=O)=O)nc(NS(=O)(=O)c2ccccc2)n1 ; O=C(O)c1ccc(Cl)c(O)c1 ; Cc1cccc(C)c1-c1cc(Oc2cc(C(=O)O)ccc2Cl)nc(NS(=O)(=O)c2ccccc2)n1]

To a 20 mL vial equipped with a magnetic stir bar, N-[4-(2,6-dimethylphenyl)-6-methylsulfonyl-pyrimidin-2-yl]benzenesulfonamide (104.0 mg, 0.2491 mmol), N-methylpyrrolidinone (3.0 mL) and 4-chloro-3-hydroxy-benzoic acid (106.8 mg, 0.6189 mmol) were added, followed by potassium carbonate (118.9 mg, 0.8603 mmol). This solution was stirred at 110° C. for 20 hours. The reaction mixture was then cooled to room temperature, quenched with 1 N HCl (4 mL), and extracted with ethyl acetate (3×4 mL). The combined organic extracts were washed with water (2×6 mL) and saturated aqueous sodium chloride solution (6 mL), then dried over sodium sulfate, filtered, and evaporated in vacuo to give a brown oil. Purification by silica gel chromatography (4 g of silica, 0 to 40% gradient of ethyl acetate/hexanes) gave a white foam, 3-[2-(benzenesulfonamido)-6-(2,6-dimethylphenyl)pyrimidin-4-yl]oxy-4-chloro-benzoic acid (97.7 mg, 74%) ESI-MS m/z calc. 509.0812, found 510.1 (M+1)⁺; Retention time: 1.77 minutes; LC method A.

### Step 5: 3-[2-(Benzenesulfonamido)-6-(2,6-dimethylphenyl)pyrimidin-4-yl]oxy-4-chloro-N-methyl-benzamide (Compound 242)

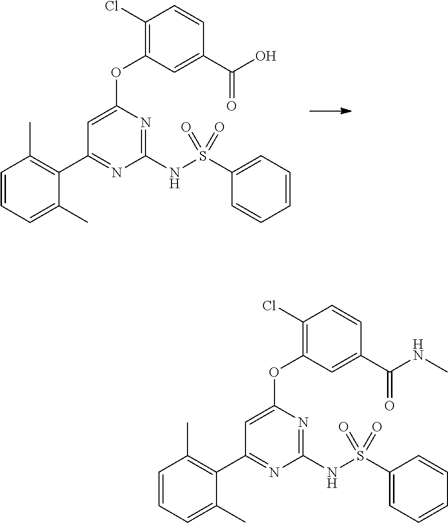 [SMILES: Cc1cccc(C)c1-c1cc(Oc2cc(C(=O)O)ccc2Cl)nc(NS(=O)(=O)c2ccccc2)n1 ; CNC(=O)c1ccc(Cl)c(Oc2cc(-c3c(C)cccc3C)nc(NS(=O)(=O)c3ccccc3)n2)c1]

To a 3 mL vial equipped with a magnetic stir bar, 3-[2-(benzenesulfonamido)-6-(2,6-dimethylphenyl)pyrimidin-4-yl]oxy-4-chloro-benzoic acid (15.94 mg, 0.0300 mmol) (15.3 mg, 0.0300 mmol), dimethylformamide (0.8 mL), methyl amine (hydrochloride salt) (6.752 mg, 0.10 mmol), diisopropylethylamine (18.55 mg, 25.00 μL, 0.1435 mmol) (25 μL, 0.14 mmol) and HATU (38.02 mg, 0.100 mmol) (38.0 mg, 0.100 mmol) were added, in this order. This mixture was stirred at room temperature for 10 minutes, after which it was filtered and purified by reverse phase HPLC (1-70% acetonitrile in water using HCl as a modifier) to give the desired product 3-[2-(benzenesulfonamido)-6-(2,6-dimethylphenyl)pyrimidin-4-yl]oxy-4-chloro-N-methyl-benzamide (10.2 mg, 65% ). ESI-MS m/z calc. 522.11285, found 523.2 (M+1)⁺; Retention time: 1.7 minutes; LC method A.

### Step 6: N-[4-[2-chloro-5-(piperazine-1-carbonyl)phenoxy]-6-(2,6-dimethylphenyl)pyrimidin-2-yl]benzenesulfonamide (Compound 244)

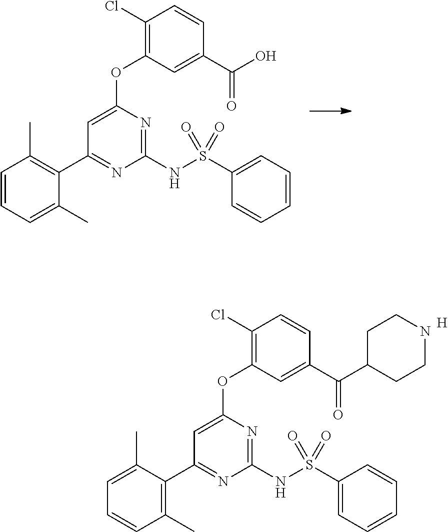

To a 3 mL vial equipped with a magnetic stir bar, 3-[2-(benzenesulfonamido)-6-(2,6-dimethylphenyl)pyrimidin-4-yl]oxy-4-chloro-benzoic acid (15.94 mg, 0.0300 mmol) (15.3 mg, 0.0300 mmol), dimethylformamide (0.8 mL), piperazine (8.614 mg, 0.10 mmol) diisopropylethylamine (18.55 mg, 25.00 μL, 0.1435 mmol) (25 μL, 0.14 mmol) and HATU (38.02 mg, 0.100 mmol) (38.0 mg, 0.100 mmol) were added, in this order. This mixture was stirred at room temperature for 10 minutes, after which it was filtered and purified by reverse phase HPLC (1-70% acetonitrile in water using HCl as a modifier) to give the desired product N-[4-[2-chloro-5-(piperazine-1-carbonyl)phenoxy]-6-(2,6-dimethylphenyl)pyrimidin-2-yl]benzenesulfonamide (hydrochloride salt) (4.9 mg, 27%)). ESI-MS m/z calc. 577.155, found 578.2 (M+1)⁺; Retention time: 1.35 minutes; LC method A.

### Step 7: 3-[2-(Benzenesulfonamido)-6-(2,6-dimethylphenyl)pyrimidin-4-yl]oxy-4-chloro-benzamide (Compound 243)

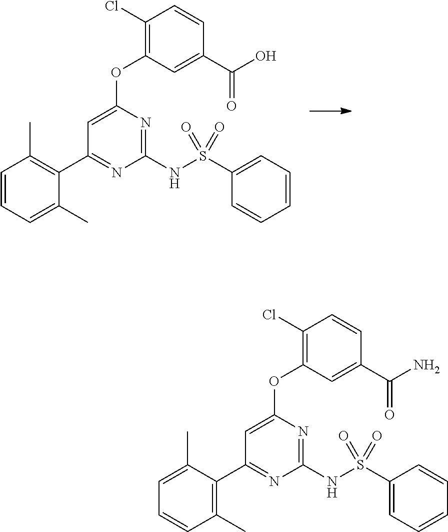 [SMILES: Cc1cccc(C)c1-c1cc(Oc2cc(C(=O)O)ccc2Cl)nc(NS(=O)(=O)c2ccccc2)n1 ; Cc1cccc(C)c1-c1cc(Oc2cc(C(N)=O)ccc2Cl)nc(NS(=O)(=O)c2ccccc2)n1]

To a 3 mL vial equipped with a magnetic stir bar, 3-[2-(benzenesulfonamido)-6-(2,6-dimethylphenyl)pyrimidin-4-yl]oxy-4-chloro-benzoic acid (15.94 mg, 0.0300 mmol) (15.3 mg, 0.0300 mmol), dimethylformamide (0.8 mL), ammonia (hydrochloride salt) (5.349 mg, 0.10 mmol) diisopropylethylamine (18.55 mg, 25.00 μL, 0.1435 mmol) (25 μL, 0.14 mmol) and HATU (38.02 mg, 0.100 mmol) (38.0 mg, 0.100 mmol) were added, in this order. This mixture was stirred at room temperature for 10 minutes, after which it was filtered and purified by reverse phase HPLC (1-70% acetonitrile in water using HCl as a modifier) to give the desired product 3-[2-(benzenesulfonamido)-6-(2,6-dimethylphenyl)pyrimidin-4-yl]oxy-4-chloro-benzamide (8 mg, 52%)). ESI-MS m/z calc. 508.0972, found 509.1 (M+1)⁺; Retention time: 1.62 minutes; LC method A.

### Example 69: Preparation of Compound 245 and Compound 246

### Step 1: 4,4,5,5-Tetramethyl-2-[2-[(Z/E)-4-methylpent-1-enyl]phenyl]-1,3,2-dioxaborolane

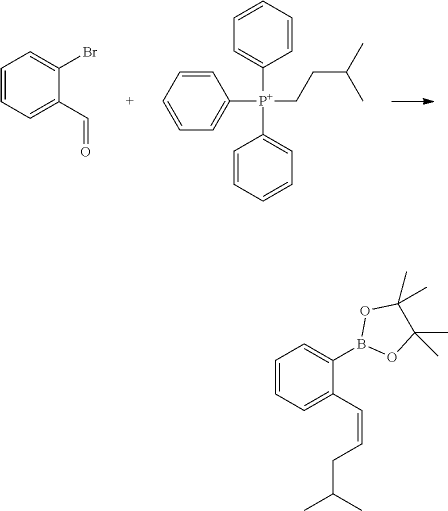 [SMILES: O=Cc1ccccc1Br ; CC(C)CC[P+](c1ccccc1)(c1ccccc1)c1ccccc1 ; CC(C)C/C=C\c1ccccc1B1OC(C)(C)C(C)(C)O1]

Stage 1: To a THE (5 mL) suspension of isopentyl(triphenyl)phosphonium bromide (1.6807 g, 4.066 mmol) at −78° C. was added nBuLi (1.5 mL of 2.5 M in hexanes, 3.750 mmol). The reaction mixture was warmed to 0° C. and stirred for 30 minutes and then treated with 2-bromobenzaldehyde (499.6 mg, 2.700 mmol). The reaction mixture was warmed to ambient temperature and stirred for 1 hour and then cooled to 0° C. and treated with HCl (4 mL of 1 M, 4.000 mmol) and diethyl ether (15 mL). The organic layer was separated, dried with anhydrous sodium sulfate, filtered, and concentrated to minimal volumes (˜2-3 mL) and then cooled to 0° C. and stirred for 15 minutes upon which the triphenylphosphine oxide precipitated out. The solid was filtered off and the filtrate was concentrated in vacuo to minimal volumes (˜2-3 mL) and the trituration was repeated once again. The ether filtrate was concentrated in vacuo to give 1-bromo-2-[(Z/E)-4-methylpent-1-enyl]benzene (1:1 mixture of isomers, 597.5 mg, 93%). ¹H NMR (400 MHz, Chloroform-d) δ 7.61-7.40 (m, 2H), 7.29-7.26 (m, 1H), 7.15-7.01 (m, 1H), 6.69 (dd, J=15.7, 1.6 Hz, 0.4H, isomer 1), 6.48 (dt, J=11.6, 1.8 Hz, 0.5H, isomer 2), 6.15 (dt, J=15.6, 7.3 Hz, 0.4H, isomer 1), 5.79 (dt, J=11.6, 7.4 Hz, 0.5H, isomer 2), 2.19-2.02 (m, 2H), 1.83-1.63 (m, 1H), 0.93 (dd, J=27.3, 6.7 Hz, 6H). (some triphenylphosphine oxide impurities in the aromatic region). ESI-MS m/z calc. 238.0357, Retention time: 0.9 minutes; LC method D.

Stage 2: 27.5 mg of the crude product from stage 1 above was taken up in dioxane (0.9 mL), water (0.1 mL), 4,4,5,5-tetramethyl-2-(4,4,5,5-tetramethyl-1,3,2-dioxaborolan-2-yl)-1,3,2-dioxaborolane (27.4 mg, 0.1079 mmol), potassium acetate (31.8 mg, 0.3240 mmol), Pd(dppf)Cl₂(8.8 mg, 0.01078 mmol) and microwaved at 120° C. for 30 minutes. The solution was filtered and the filtrate dissolved in 0.8 mL MeOH, and purified by reverse phase chromatography using a 15 minutes gradient of 50% MeCN in water to 99% MeCN (HCl modifier) to give 4,4,5,5-tetramethyl-2-[2-[(Z/E)-4-methylpent-1-enyl]phenyl]-1,3,2-dioxaborolane (1:1 mixture of isomers, 19.1 mg, 2%) ESI-MS m/z calc. 286.21042, found 287.1 (M+1)⁺; Retention time: 2.5 minutes; LC method A.

### Step 2: 1-Methyl-N-[4-[2-[(E)-4-methylpent-1-enyl]phenyl]-6-[4-(4-methylpiperazin-1-yl)phenoxy]pyrimidin-2-yl]pyrazole-4-sulfonamide (Compound 245) and N-[4-(2-isohexylphenyl)-6-[4-(4-methylpiperazin-1-yl)phenoxy]pyrimidin-2-yl]-1-methyl-pyrazole-4-sulfonamide (Compound 246)

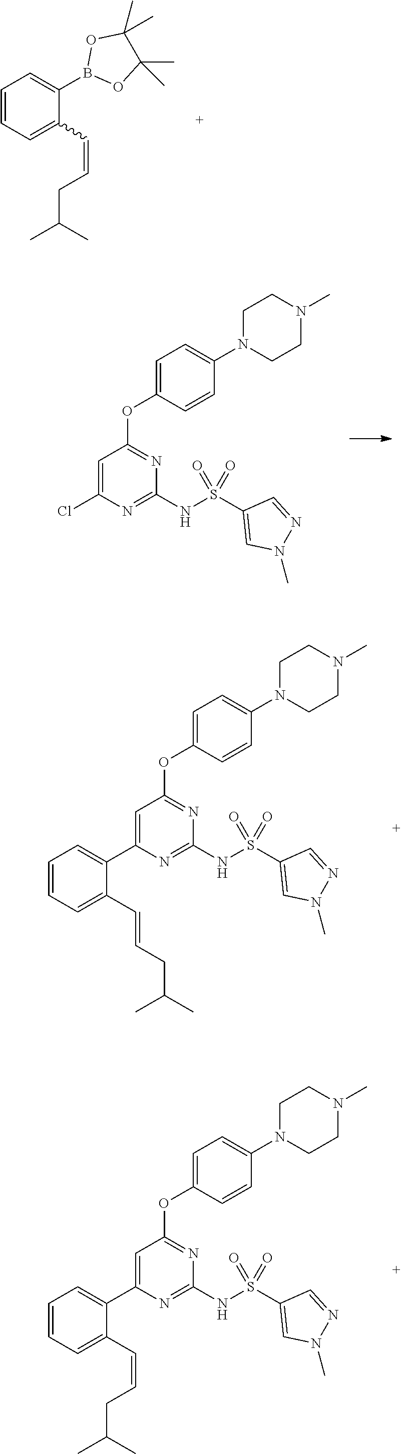 [SMILES: CC(C)CC=Cc1ccccc1B1OC(C)(C)C(C)(C)O1 ; CN1CCN(c2ccc(Oc3cc(Cl)nc(NS(=O)(=O)c4cnn(C)c4)n3)cc2)CC1 ; CC(C)C/C=C/c1ccccc1-c1cc(Oc2ccc(N3CCN(C)CC3)cc2)nc(NS(=O)(=O)c2cnn(C)c2)n1 ; CC(C)C/C=C\c1ccccc1-c1cc(Oc2ccc(N3CCN(C)CC3)cc2)nc(NS(=O)(=O)c2cnn(C)c2)n1]

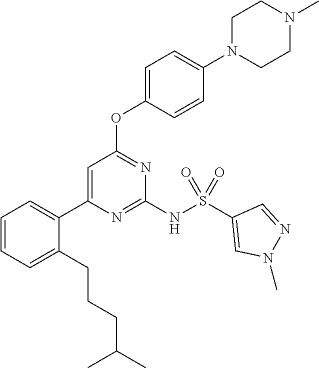 [SMILES: CC(C)CCCc1ccccc1-c1cc(Oc2ccc(N3CCN(C)CC3)cc2)nc(NS(=O)(=O)c2cnn(C)c2)n1]

Stage 1: A dioxane (0.9 mL) mixture of N-[4-chloro-6-[4-(4-methylpiperazin-1-yl)phenoxy]pyrimidin-2-yl]-1-methyl-pyrazole-4-sulfonamide (48.1 mg, 0.1037 mmol) 4,4,5,5-tetramethyl-2-[2-[(Z/E)-4-methylpent-1-enyl]phenyl]-1,3,2-dioxaborolane (1:1 mixture of isomers, 46.2 mg, 0.1614 mmol), K₂CO₃ (100 μL of 2 M, 0.2000 mmol), and Pd(PPh₃)₄ (21.7mg, 0.01878 mmol) was microwaved at 120° C. for 30 minutes. The solution was filtered, and the filtrate was diluted with 0.8 mL MeOH. This solution was separated into two equal portions. The first portion was purified by reverse phase chromatography using a 15 minutes gradient of 1% MeCN in water to 99% MeCN (HCl modifier) to give:

The trans alkene, 1-methyl-N-[4-[2-[(E)-4-methylpent-1-enyl]phenyl]-6-[4-(4-methylpiperazin-1-yl)phenoxy]pyrimidin-2-yl]pyrazole-4-sulfonamide (hydrochloride salt) (4.1 mg, 6%) ESI-MS m/z calc. 587.2679, found 588.5 (M+1)⁺; Retention time: 1.54 minutes; LC method A, and the cis alkene, 1-methyl-N-[4-[2-[(Z)-4-methylpent-1-enyl]phenyl]-6-[4-(4-methylpiperazin-1-yl)phenoxy]pyrimidin-2-yl]pyrazole-4-sulfonamide (hydrochloride salt) (4.6 mg, 7%) ESI-MS m/z calc. 587.2679, found 588.4 (M+1)⁺; Retention time: 1.51 minutes; LC method A. The second portion was also purified but kept aside for Stage 2.

Stage 2: The products set aside from Stage 1 were dissolved in EtOH (1 mL) and sparged with nitrogen for 5 minutes and then treated with Pd/C (32.1 mg, 0.03016 mmol) and further sparged with nitrogen for 5 minutes. The reaction is then hydrogenated under an atmosphere of hydrogen (30 mg, 14.88 mmol) using a hydrogen balloon for 2 hours. The catalyst is filtered, and the filtrate was concentrated in vacuo to give N-[4-(2-isohexylphenyl)-6-[4-(4-methylpiperazin-1-yl)phenoxy]pyrimidin-2-yl]-1-methyl-pyrazole-4-sulfonamide (hydrochloride salt) (7.7 mg, 88%) ESI-MS m/z calc. 589.2835, found 590.37 (M+1)⁺; Retention time: 1.67 minutes; LC method A.

### Example 70: Preparation of Compound 247

### Step 1: 1-Bromo-2-[(Z)-1,3-dimethylbut-1-enyl]benzene

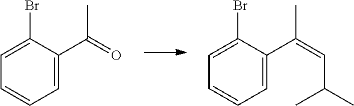

To a THE (12 mL) mixture of isobutyl(triphenyl)phosphonium bromide (3.507 g, 8.783 mmol) under nitrogen at 0° C. was added solid potassium tert-butoxide (987.7 mg, 8.802 mmol) in one portion. The mixture was warmed to room temperature and stirred for 1 hour and then cooled back down to 0° C. 1-(2-Bromophenyl)ethanone (500 mg, 2.512 mmol) was added and the reaction mixture was warmed to room temperature and stirred for 16 hours and then at 50° C. for 8 hours. The reaction mixture was cooled to 0° C. and treated with HCl (10 mL of 1 M, 10.00 mmol) and then diluted with diethyl ether (20 mL). The organic layer was separated and washed with water (10 mL) and then brine (10 mL). The organic layer was dried over sodium sulfate, filtered, and concentrated in vacuo. The resulting residue is taken up in diethyl ether (approximately 3 mL) and cooled to 0° C. for 15 minutes and then filtered. The filtrate was concentrated in vacuo and the trituration was repeated. The filtrate was concentrated in vacuo and then loaded onto a short silica plug, eluting with diethyl ether to give 1-bromo-2-[(Z)-1,3-dimethylbut-1-enyl]benzene (10 mg, 1%) ¹H NMR (400 MHz, Chloroform-d) δ 7.60-7.53 (m, 1H), 7.29-7.23 (m, 1H), 7.15-7.00 (m, 2H), 5.30 (d, J=9.9, 1.5 Hz, 1H), 2.00-1.90 (m, 4H), 0.89 (dd, J=21.1, 6.6 Hz, 6H). (Some triphenylphosphine oxide byproduct present in aromatic region).

### Step 2: 2-[2-[(Z)-1,3-dimethylbut-1-enyl]phenyl]-4,4,5,5-tetramethyl-1,3,2-dioxaborolane

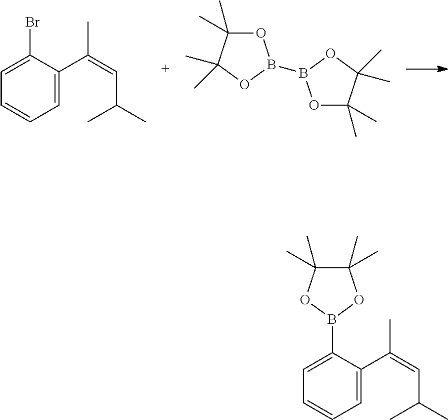

A dioxane (0.9 mL) and water (0.1 mL) mixture of 1-bromo-2-[(Z)-1,3-dimethylbut-1-enyl]benzene (10 mg, 0.04181 mmol), 4,4,5,5-tetramethyl-2-(4,4,5,5-tetramethyl-1,3,2-dioxaborolan-2-yl)-1,3,2-dioxaborolane (22.3 mg, 0.08782 mmol), potassium acetate (12.6 mg, 0.1284 mmol), and Pd(PPh₃)₄ (11.7 mg, 0.01012 mmol) was microwaved at 120° C. for 30 minutes and then cooled to room temperature. The solution was filtered and the filtrate diluted with 0.8 mL of MeOH, and purified by reverse phase chromatography using a 15 min gradient of 30% MeCN in water to 99% MeCN (HCl modifier) to give 2-[2-[(Z)-1,3-dimethylbut-1-enyl]phenyl]-4,4,5,5-tetramethyl-1,3,2-dioxaborolane (4 mg, 33%) ESI-MS m/z calc. 286.21042, found 287.2 (M+1)⁺; Retention time: 1.59 minutes; LC method A.

### Step 3: N-[4-[2-[(Z)-1,3-dimethylbut-1-enyl]phenyl]-6-[4-(4-methylpiperazin-1-yl)phenoxy]pyrimidin-2-yl]-1-methyl-pyrazole-4-sulfonamide

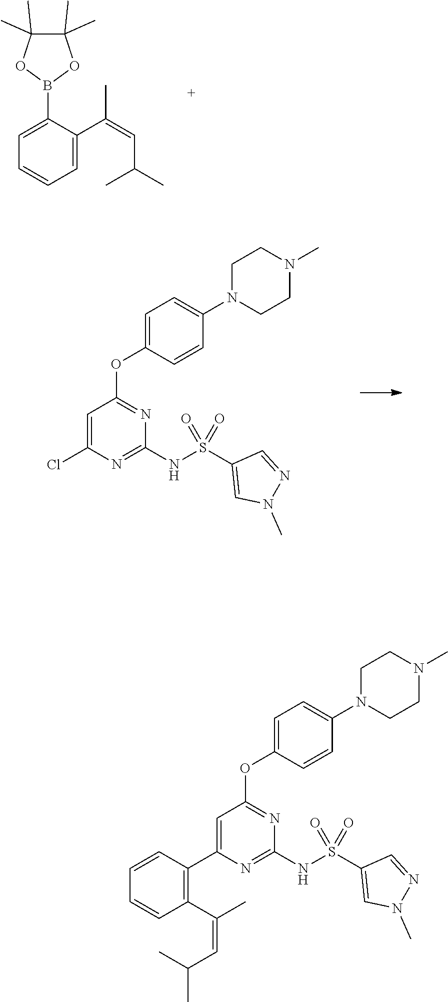 [SMILES: C/C(=C/C(C)C)c1ccccc1B1OC(C)(C)C(C)(C)O1 ; "+" ; CN1CCN(c2ccc(Oc3cc(Cl)nc(NS(=O)(=O)c4cnn(C)c4)n3)cc2)CC1 ; C/C(=C/C(C)C)c1ccccc1-c1cc(Oc2ccc(N3CCN(C)CC3)cc2)nc(NS(=O)(=O)c2cnn(C)c2)n1]

The compound was prepared in a manner analogous Compound 245 and Compound 246 described above.

### Step 4: N-[4-[2-(1,3-dimethylbutyl)phenyl]-6-[4-(4-methylpiperazin-1-yl)phenoxy]pyrimidin-2-yl]-1-methyl-pyrazole-4-sulfonamide (Compound 247)

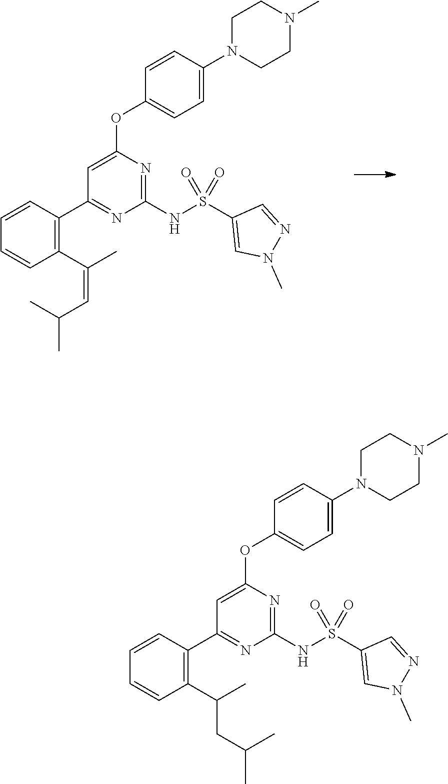 [SMILES: C/C(=C/C(C)C)c1ccccc1-c1cc(Oc2ccc(N3CCN(C)CC3)cc2)nc(NS(=O)(=O)c2cnn(C)c2)n1 ; CC(C)CC(C)c1ccccc1-c1cc(Oc2ccc(N3CCN(C)CC3)cc2)nc(NS(=O)(=O)c2cnn(C)c2)n1]

To a solution of N-[4-[2-[(Z)-1,3-dimethylbut-1-enyl]phenyl]-6-[4-(4-methylpiperazin-1-yl)phenoxy]pyrimidin-2-yl]-1-methyl-pyrazole-4-sulfonamide (6 mg, 0.01021 mmol) in EtOH (0.5 mL) was added Pd/C (2 mg of 10% w/w, 0.001879 mmol) and the reaction mixture was stirred at 40° C. for 3 hours. The reaction mixture was diluted with MeOH, filtered and purification by HPLC (1-99% ACN in water (HCl modifier)) gave N-[4-[2-(1,3-dimethylbutyl)phenyl]-6-[4-(4-methylpiperazin-1-yl)phenoxy]pyrimidin-2-yl]-1-methyl-pyrazole-4-sulfonamide (hydrochloride salt) (0.6 mg, 9%). ESI-MS m/z calc. 589.2835, found 590.4 (M+1)⁺; Retention time: 1.51 minutes; LC method A.

### Example 71: Preparation of Compound 248

### Step 1: N-[4-(2-isobutoxy-6-methyl-phenyl)-6-[4-(4-methylpiperazin-1-yl)phenoxy]pyrimidin-2-yl]-1-methyl-pyrazole-4-sulfonamide (Compound 248)

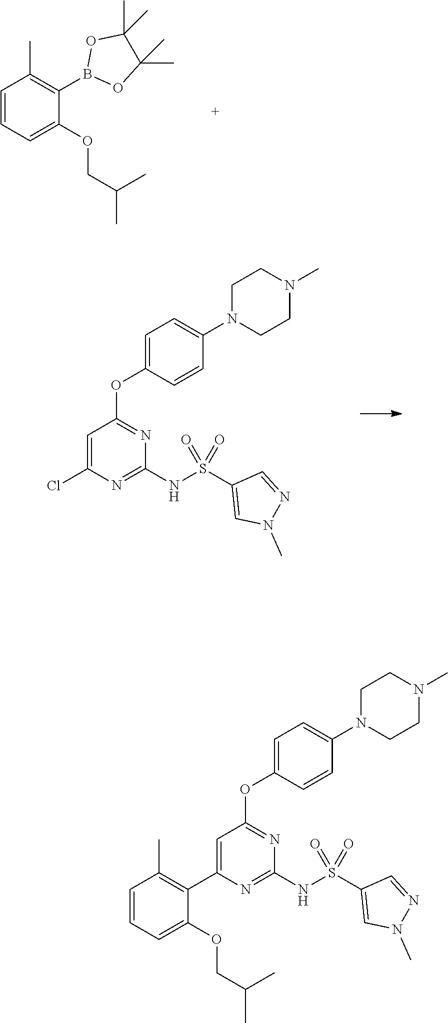 [SMILES: Cc1cccc(OCC(C)C)c1B1OC(C)(C)C(C)(C)O1 ; "+" ; CN1CCN(c2ccc(Oc3cc(Cl)nc(NS(=O)(=O)c4cnn(C)c4)n3)cc2)CC1 ; Cc1cccc(OCC(C)C)c1-c1cc(Oc2ccc(N3CCN(C)CC3)cc2)nc(NS(=O)(=O)c2cnn(C)c2)n1]

A dioxane (0.9 mL) mixture of 2-(2-isobutoxy-6-methyl-phenyl)-4,4,5,5-tetramethyl-1,3,2-dioxaborolane (80.2 mg, 0.2764 mmol), N-[4-chloro-6-[4-(4-methylpiperazin-1-yl)phenoxy]pyrimidin-2-yl]-1-methyl-pyrazole-4-sulfonamide (hydrochloride salt) (100.1 mg, 0.2000 mmol), Pd(PPh₃)₄ (25.1 mg, 0.02172 mmol) and K₂CO₃ (100 μL of 2 M, 0.2000 mmol) was microwaved at 120° C. for 30 minutes and then cooled to room temperature. The solution was filtered and the filtrate dissolved in 0.8 mL MeOH, and purified by reverse phase chromatography using a 15 minute gradient of 1% MeCN in water to 99% MeCN (HCl modifier) to give N-[4-(2-isobutoxy-6-methyl-phenyl)-6-[4-(4-methylpiperazin-1-yl)phenoxy]pyrimidin-2-yl]-1-methyl-pyrazole-4-sulfonamide (hydrochloride salt) (3.7 mg, 3%) ESI-MS m/z calc. 591.26276, found 592.3 (M+1)⁺; Retention time: 1.41 minutes; LC method A.

### Example 72: Preparation of Compound 249

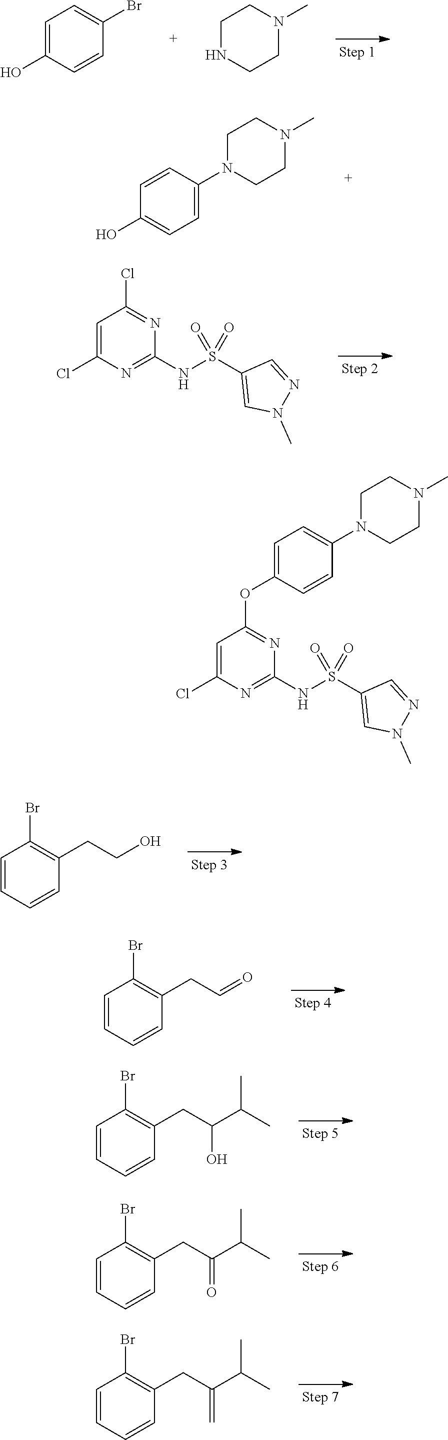

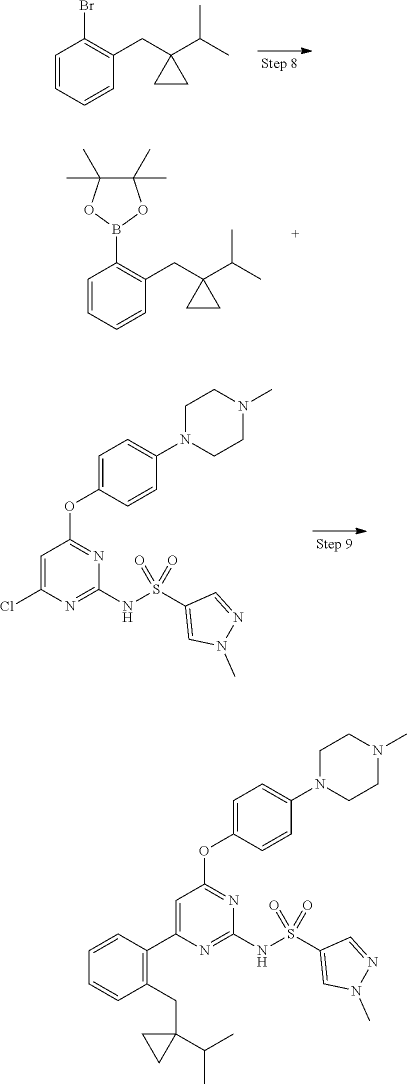

### Step 1: 4-(4-methylpiperazin-1-yl)phenol

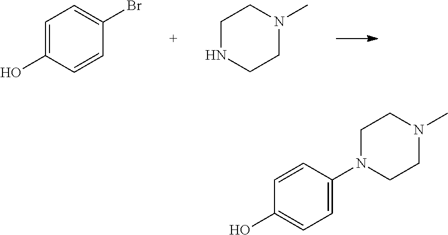

In a 500 mL three-neck round bottom flask were combined 4-bromophenol (15.2141 g, 87.94 mmol) and [2-(2-aminoethyl)phenyl]-chloro-palladium;ditert-butyl-[2-(2,4,6-triisopropylphenyl)phenyl]phosphane (XPhos Pd G1)(3.44 g, 5.010 mmol) and the solid mixture was purged under nitrogen for 75 minutes. In a separate flask was 1-methylpiperazine (60 mL, 540.3 mmol) and dioxane (200 mL) were combined and the mixture was sparged with nitrogen for 30 minutes and then cannulated into the 500 mL three-neck round bottom flask under nitrogen pressure. Sodium tert-butoxide (18.18 g, 189.2 mmol) was added and the reaction was stirred for 16 hours at room temperature. HCl (80 mL of 2 M, 160.0 mmol) was added and the internal temperature was maintained at −22-24° C. Keep the pH basic. The mixture was poured into 500 mL of DCM and the phases were separated. The product was extracted from the aqueous layer with 200 mL DCM. DCM layers were combined and dried over anhydrous magnesium sulfate, filtered, and concentrated in vacuo. The brown residue was taken up in ethyl acetate (300 mL), heated to 60° C. and stirred for 20 minutes, then cooled to room temperature and filtered. The wet cake was washed with ethyl acetate (30 mL) and then diethyl ether (100 mL) and dried under vacuum suction for 30 minutes to give 4-(4-methylpiperazin-1-yl)phenol (9.8378 g, 58%). ¹H NMR (400 MHz, DMSO-d₆) δ 8.79 (s, 1H), 6.76 (d, J=9.0 Hz, 2H), 6.67 -6.58 (m, 2H), 2.93 (dd, J=6.1, 3.8 Hz, 4H), 2.45-2.39 (m, 4H), 2.20 (s, 3H). ESI-MS m/z calc. 192.12627, found 193.25 (M+1)⁺; Retention time: 0.37 minutes.

### Step 2: N-[4-chloro-6-[4-(4-methylpiperazin-1-yl)phenoxy]pyrimidin-2-yl]-1 -methyl-pyrazole-4-sulfonamide

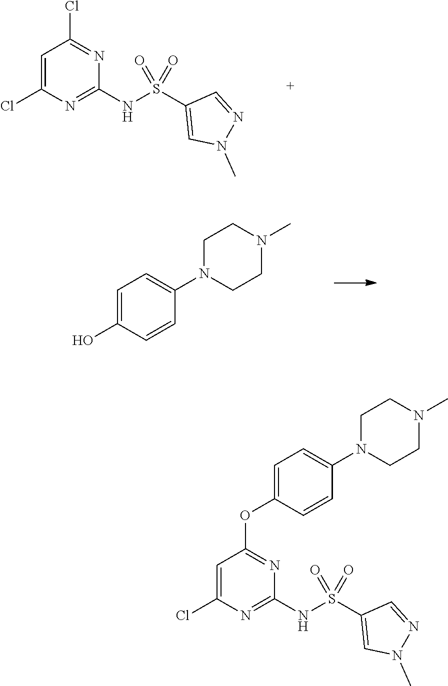 [SMILES: Cn1cc(S(=O)(=O)Nc2nc(Cl)cc(Cl)n2)cn1 ; "+" ; CN1CCN(c2ccc(O)cc2)CC1 ; CN1CCN(c2ccc(Oc3cc(Cl)nc(NS(=O)(=O)c4cnn(C)c4)n3)cc2)CC1]

A NMP (6 mL) mixture of N-(4,6-dichloropyrimidin-2-yl)-1-methyl-pyrazole-4-sulfonamide (992.0 mg, 3.123 mmol), 4-(4-methylpiperazin-1-yl)phenol (399.7 mg, 2.079 mmol), and Cs₂CO₃ (2.723 g, 8.357 mmol) was heated to 100° C. for 15 minutes and then cooled down to room temperature and filtered to remove the salts. The filtrate was loaded onto a 15.5 g redisep C₁₈ column and purified by reverse phase chromatography with an eluent of 0-60% Acetonitrile/0.05% Formic acid in H₂O over 25 minutes to give N-[4-chloro-6-[4-(4-methylpiperazin-1-yl)phenoxy]pyrimidin-2-yl]-1-methyl-pyrazole-4-sulfonamide (formic acid salt) (572.5 mg, 54%). ESI-MS m/z calc. 463.11932, found 464.0 (M+1)⁺; Retention time: 0.91 minutes; LC method A.

### Step 3: 2-(2-bromophenyl)acetaldehyde

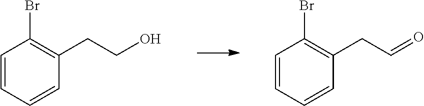 [SMILES: OCCc1ccccc1Br ; O=CCc1ccccc1Br]

To a solution of 2-(2-bromophenyl)ethanol (11.85 g. 58.90 mmol) in dichloromethane (200 mL) was added Dess-Martin periodinane (30.0 g, 70.7 mmol) in several batches at room temperature. The reaction mixture was stirred at room temperature for 3 hours. The reaction was filtered through a pad of Celite. The filtrate was diluted with 10% sodium bicarbonate aqueous solution (200 mL). Two layers were separated. The organic layer was washed with 10% sodium bicarbonate aqueous solution (200 mL) and brine (200 mL), dried over anhydrous magnesium sulfate and concentrated under reduced pressure. The residue was purified by silica gel chromatography using 0 to 30% hexane-dichloromethane to furnish 2-(2-bromophenyl)acetaldehyde (9.87 g, 84%) as a light yellow liquid. ¹H NMR (250 MHz, CDCl₃) δ (ppm): 9.77 (t, J=1.8 Hz, 1H), 7.61 (dd, J=7.7 Hz, 1.1 Hz, 1H), 7.40-7.10 (m, 3H), 3.88 (s, 2H).

### Step 4: 1-(2-bromophenyl)-3-methylbutan-2-ol

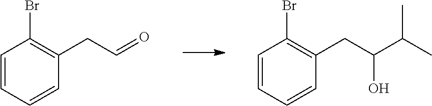

To a solution of 2-(2-bromophenyl)acetaldehyde (9.19 g, 46.17 mmol) in anhydrous diethyl ether (250 mL) was added isopropylmagnesium chloride (2.0 M in tetrahydrofuran solution, 46 mL, 92.34 mmol) at −5° C. The reaction was stirred at 0° C. for 1 hour. The reaction was quenched with 10% ammonium chloride aqueous solution (250 mL). Two layers were separated. The aqueous layer was extracted with diethyl ether (2×250 mL). The combined organic layers were washed with brine (250 mL) and dried over anhydrous magnesium sulfate and concentrated under vacuum. The crude product was purified by silica gel chromatography using 0 to 30% hexane-diethyl ether to furnish 1-(2-bromophenyl)-3-methylbutan-2-ol (5.43 g, 48%) as a white solid. ESI-MS m/z: calc. 242.03, found (M+1)⁺. Retention time: 5.10 minutes.

¹H NMR (250 MHz, CDCl₃) δ (ppm): 7.54 (d, J=7.9 Hz, 1H), 7.29 (m, 2H), 7.10 (td, J=8.7 Hz, 2.6 Hz, 1H), 3.69 (m, 1H), 3.07 (dd, J=13.7 Hz, 2.9 Hz, 1H), 2.68 (dd, J=13.6 Hz, 9.8 Hz, 1H), 1.80 (m, 1H), 1.05 (d, J=6.7 Hz, 6H).

### Step 5: 1-(2-bromo-phenyl)-3-methyl-butan-2-one

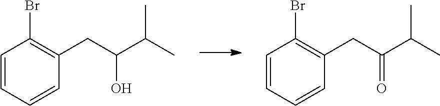

Into a solution of 1-(2-bromophenyl)-3-methylbutan-2-ol (5.43 g, 22.33 mmol) in dichloromethane (100 mL) was added Dess-Martin periodinane (11.37 g, 26.80 mmol). The reaction mixture was stirred at room temperature for 1 hour. The precipitate was removed by filtration. The filtrate was washed with saturated sodium bicarbonate aqueous solution (2×100 mL), and brine (100 mL), dried over anhydrous sodium sulfate and concentrated under vacuum. The residue was purified by silica gel chromatography using 0 to 10% hexane-ethyl acetate to furnish 1-(2-bromo-phenyl)-3-methyl-butan-2-one (4.64 g, 86%) as a clear liquid. ESI-MS m/z: calc. 240.01, found (M+1)⁺. Retention time: 5.18 minutes. ¹H NMR (250 MHz, CDCl₃) δ (ppm): 7.55 (d, J=7.8 Hz, 1H), 7.35-7.05 (m, 3H), 3.93 (s, 2H), 2.77 (m, 1H), 1.16 (d, J=7.0 Hz, 6H).

### Step 6: 1-bromo-2-(3-methyl-2-methylenebutyl)benzene

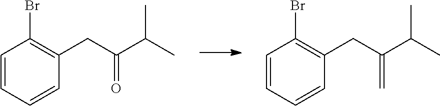

Into a 1-L round bottom flask, was placed activated zinc powder (26.7 g, 0.411 mol) and anhydrous tetrahydrofuran (250 mL). Dibromomethane (10.1 mL, 0.144 mol) was added to the reaction mixture. Titanium tetrachloride (11.5 mL, 0.103 mol) was added to the reaction mixture at −40° C. (dry ice-acetonitrile bath) within 1 hour. The reaction was then stirred at 0 to 5° C. for 60 hours. The dark gray slurry was cooled with an ice batch and anhydrous dichloromethane (50 mL) was added. To the stirred solution was added a solution of 1-(2-bromophenyl)-3-methylbutan-2-one (4.23 g, 17.54 mmol) in anhydrous dichloromethane (50 mL) at 0° C. over 10 minutes. The reaction mixture was stirred at room temperature for 2 hours. The mixture was diluted with pentane (300 mL) and a slurry of sodium bicarbonate (150 g) in water (80 mL) was added cautiously over 1 hour. The clear organic solution was washed with saturated sodium bicarbonate aqueous solution (2×200 mL), and brine (200 mL), dried over anhydrous magnesium sulfate and concentrated under vacuum. The residue was purified by silica gel chromatography using pentane to furnish 1-bromo-2-(3-methyl-2-methylenebutyl)benzene (2.755 g, 66%) as a clear oil. ESI-MS m/z: calc. 238.04, found (M+1)⁺. Retention time: 7.33 minutes. ¹H NMR (250 MHz, DMSO-d₆) δ (ppm): 7.58 (d, J=7.5 Hz, 1H), 7.30 (m, 2H), 7.18 (m, 1H), 4.83 (s, 1H), 4.28 (s, 1H), 3.45 (s, 2H), 2.26 (m, 1H), 1.07 (d, J=5.0 Hz, 6H).

### Step 7: 1-Bromo-2-((1-isopropylcyclopropyl)methyl)benzene

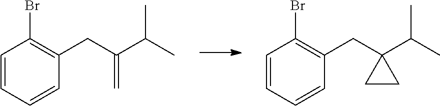

A round bottom flask was charged with anhydrous dichloromethane (50 mL). Diethylzine (1.0 M in hexane, 57.6 mL, 57.6 mmol) was added at 0° C. After 10 minutes, a solution of trifluoroacetic acid (4.41 mL, 57.6 mmol) in dichloromethane (50 mL) was added dropwise. The reaction mixture was stirred at 0° C. for 20 minutes. A solution of diiodomethane (4.65 mL, 57.6 mmol) in dichloromethane (50 mL) was added at 0° C. The reaction was stirred at 0° C. for another 20 minutes. A solution of 1-bromo-2-(3-methyl-2-methylenebutyl)benzene (2.755 g, 11.52 mmol) in dichloromethane (60 mL) was added to the reaction mixture at 0° C. The reaction was stirred at ambient temperature for 1 hour, and then it was quenched with 1 N hydrochloric acid (100 mL) and diluted with pentane (100 mL). The two layers were separated. The aqueous layer was extracted with pentane (2×100 mL). The combined organic layers were washed with saturated sodium thiosulfate (100 mL) and brine (100 mL), dried over anhydrous sodium sulfate and concentrated under vacuum. The residue was purified by silica gel chromatography using hexane to furnish 1-bromo-2-((1-isopropylcyclopropyl)methyl)benzene (3.019 g, quantitative) as a pink oil. ESI-MS m/z: calc. 252.05, found (M+1)⁺. Retention time: 7.76 minutes. ¹H NMR (250 MHz, CDCl₃) δ (ppm): 7.49 (d, J=7.9 Hz, 1H), 7.19 (m, 2H), 7.05 (m, 1H), 2.91 (s, 2H), 1.26 (m, 1H), 1.02 (d, J=6.7 Hz, 6H), 0.24 (m, 2H), 0.10 (m, 2H).

### Step 8: 2-[2-(1-Isopropyl-cyclopropylmethyl)-phenyl]-4,4,5,5-tetramethyl-[1,3,2]dioxaborolane

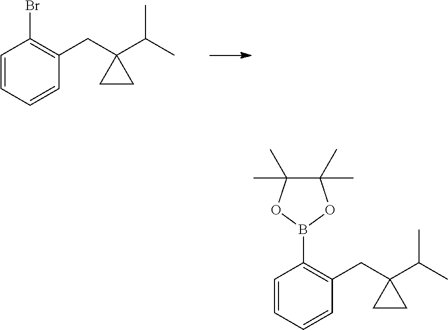 [SMILES: CC(C)C1(Cc2ccccc2Br)CC1 ; CC(C)C1(Cc2ccccc2B2OC(C)(C)C(C)(C)O2)CC1]

Into a solution of 1-bromo-2-(1-isopropyl-cyclopropylmethyl)-benzene (3.019 g, 11.92 mmol) in anhydrous dioxane (50 mL) was added potassium acetate (3.509 g, 35.76 mmol) and bis(pinacolato)diboron (4.542 g, 17.89 mmol). The reaction was purged with nitrogen, then 1,1-bis(diphenylphosphino)ferrocene palladium (II) dichloride dichloromethane (0.487 g, 0.596 mmol) was added. The reaction was refluxed under argon for 16 hours. The reaction was quenched with water (50 mL) and extracted with ethyl acetate (3×50 mL). The combined organic layers were washed with brine (2×50 mL), dried over anhydrous sodium sulfate and concentrated under vacuum. The residue was purified by silica gel chromatography using 0 to 20% hexane-dichloromethane to furnish 2-[2-(1-Isopropyl-cyclopropylmethyl)-phenyl]-4,4,5,5-tetramethyl-[1,3,2]dioxaborolane (2.711 g, 76%) as a clear liquid. ESI-MS m/z: calc. 300.23, found 301.0 (M+1)⁺. Retention time: 8.72 minutes. ¹H NMR (250 MHz, CDCl₃) δ(ppm): 7.72 (d, J=7.2 Hz, 1H), 7.28 (m, 1H), 7.14 (m, 2H), 3.10 (s, 2H), 1.35 (s, 12H), 1.20 (m, 1H), 1.03 (d, J=6.4 Hz, 6H), 0.09 (m, 2H), -0.02 (m, 2H).

### Step 9: N-[4-[2-[(1-isopropylcyclopropyl)methyl]phenyl]-6-[4-(4-methylpiperazin-1-yl)phenoxy]pyrimidin-2-yl]-1-methyl-pyrazole-4-sulfonamide (Compound 249)

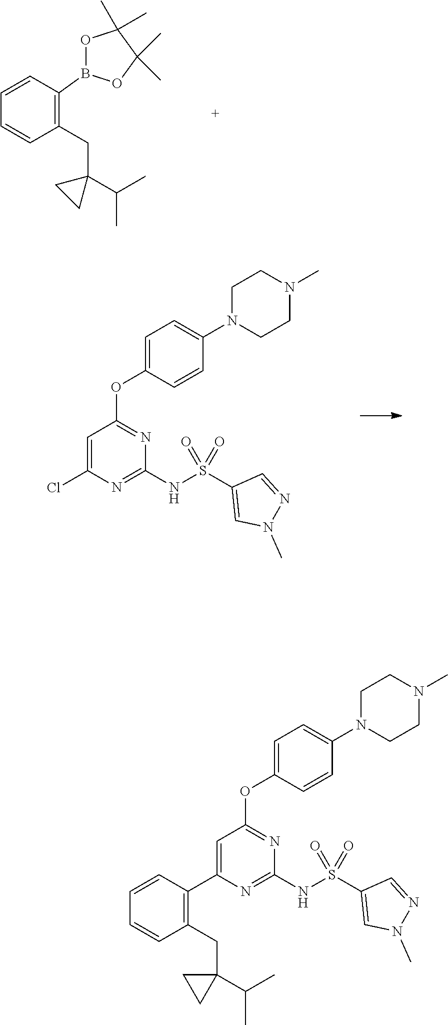 [SMILES: CC(C)C1(Cc2ccccc2B2OC(C)(C)C(C)(C)O2)CC1 ; "+" ; CN1CCN(c2ccc(Oc3cc(Cl)nc(NS(=O)(=O)c4cnn(C)c4)n3)cc2)CC1 ; CC(C)C1(Cc2ccccc2-c2cc(Oc3ccc(N4CCN(C)CC4)cc3)nc(NS(=O)(=O)c3cnn(C)c3)n2)CC1]

A dioxane (0.9 mL) mixture of 2-[2-[(1-isopropylcyclopropyl)methyl]phenyl]-4,4,5,5-tetramethyl-1,3,2-dioxaborolane (19.3 mg, 0.06428 mmol), N-[4-chloro-6-[4-(4-methylpiperazin-1-yl)phenoxy]pyrimidin-2-yl]-1-methyl-pyrazole-4-sulfonamide (formic acid salt) (24.6 mg, 0.04824 mmol), Pd(PPh₃)₄ (10.1 mg, 0.008740 mmol), and K₂CO₃ (100 μL of 2 M, 0.2000 mmol) was sparged with nitrogen for 1 minute and then microwaved at 120° C. for 50 minutes. The solution was filtered and the filtrate purified by reverse phase chromatography using a 15 minute gradient of 1% MeCN in water to 99% MeCN with ammonium formate modifier and the pure fractions were concentrated in vacuo by rotary evaporation to give N-[4-[2-[(1-isopropylcyclopropyl)methyl]phenyl]-6-[4-(4-methylpiperazin-1-yl)phenoxy]pyrimidin-2-yl]-1-methyl-pyrazole-4-sulfonamide (formic acid salt) (9 mg, 29%). ¹H NMR (400 MHz, DMSO-d₆) δ 8.17 (s, 1H), 7.68 (s, 1H), 7.45-7.22 (m, 5H), 7.13-6.97 (m, 4H), 6.46 (s, 1H), 3.72 (s, 3H), 3.15 (t, J=5.0 Hz, 4H), 2.88 (s, 2H), 2.48-2.44 (m, 4H), 2.23 (s, 3H), 1.03 (p, J=6.9 Hz, 1H), 0.64 (d, J=6.8 Hz, 6H), 0.13-0.02 (m, 2H), -0.07 --0.17 (m, 2H). ESI-MS m z calc. 601.2835, found 602.4 (M+1)⁺; Retention time: 1.56 minutes; LC method A.

### Example 73: Preparation of Compound 250

### Step 1: 2-(2-Isohexyl-3-methoxy-phenyl)-4,4,5,5-tetramethyl-1,3,2-dioxaborolane

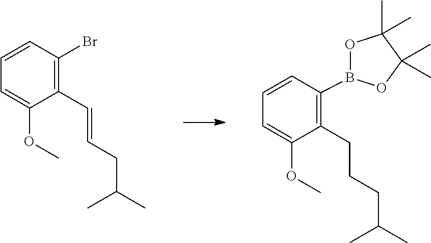 [SMILES: COc1cccc(Br)c1/C=C/CC(C)C ; COc1cccc(B2OC(C)(C)C(C)(C)O2)c1CCCC(C)C]

A mixture of 1-bromo-3-methoxy-2-[(E)-4-methylpent-1-enyl]benzene (38.5 mg, 0.1430 mmol), acetate (Potassium Ion (1)) (41 mg, 0.4178 mmol), 4,4,5,5-tetramethyl-2-(4,4,5,5-tetramethyl-1,3,2-dioxaborolan-2-yl)-1,3,2-dioxaborolane (43.6 mg, 0.1717 mmol), water (0.3 mL)and Pd(dppf)C₁₂(3.2 mg, 0.003918 mmol) in dioxane (3 mL) was heated at 120° C. for 20 minutes and then cooled to room temperature. The solution was filtered and the filtrate dissolved in 0.8 mL MeOH, and purified by reverse phase chromatography using a 15 minute gradient of 30% MeCN in water to 99% MeCN (HCl modifier) to give 2-[3-methoxy-2-[(E)-4-methylpent-1-enyl]phenyl]-4,4,5,5-tetramethyl-1,3,2-dioxaborolane (37.2 mg, 82%) ESI-MS m/z calc. 316.22098, found 317.2 (M+1)⁺; Retention time: 2.38 minutes; LC method A.

The product from above 2-[3-methoxy-2-[(E)-4-methylpent-1-enyl]phenyl]-4,4,5,5-tetramethyl-1,3,2-dioxaborolane (37.2 mg, 82%) was taken up in EtOH (1 mL) and treated with palladium (17.6 mg, 0.01654 mmol). The reaction was evacuated and back-filled with nitrogen, and stirred at room temperature for 2 hours under an atmosphere of hydrogen (800 mg, 396.8 mmol)(balloon), then filtered over Celite and concentrated in vacuo to give 2-(2-isohexyl-3-methoxy-phenyl)-4,4,5,5-tetramethyl-1,3,2-dioxaborolane (35.4 mg, 78%) ESI-MS m/z calc. 318.23663, found 319.3 (M+1)⁺; Retention time: 1.63 minutes; LC method A.

### Step 2: N-[4-(2-Isohexyl-3-methoxy-phenyl)-6-[4-(4-methylpiperazin-1-yl)phenoxy]pyrimidin-2-yl]-1-methyl-pyrazole-4-sulfonamide (Compound 250)

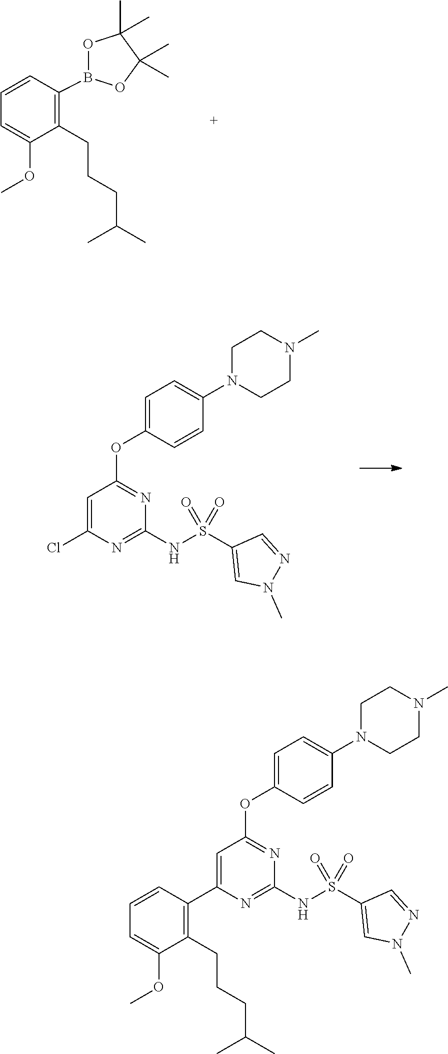 [SMILES: COc1cccc(B2OC(C)(C)C(C)(C)O2)c1CCCC(C)C ; "+" ; CN1CCN(c2ccc(Oc3cc(Cl)nc(NS(=O)(=O)c4cnn(C)c4)n3)cc2)CC1 ; COc1cccc(-c2cc(Oc3ccc(N4CCN(C)CC4)cc3)nc(NS(=O)(=O)c3cnn(C)c3)n2)c1CCCC(C)C]

A dioxane (0.9 mL) mixture of 2-(2-isohexyl-3-methoxy-phenyl)-4,4,5,5-tetramethyl-1,3,2-dioxaborolane (19.4 mg, 0.06096 mmol), Pd(PPh₃)₄ (11.3 mg, 0.009779 mmol), N-[4-chloro-6-[4-(4-methylpiperazin-1-yl)phenoxy]pyrimidin-2-yl]-1-methyl-pyrazole-4-sulfonamide (formic acid salt) (20.4 mg, 0.04000 mmol), and K₂CO₃ (100 μL of 2 M, 0.2000 mmol) was microwaved at 120° C. for 20 minutes. The solution was filtered and the filtrate diluted with 0.8 mL MeOH, and purified by reverse phase chromatography using a 15 min gradient of 20% MeCN in water to 80% MeCN (HCl modifier) to give N-[4-(2-isohexyl-3-methoxy-phenyl)-6-[4-(4-methylpiperazin-1-yl)phenoxy]pyrimidin-2-yl]-1-methyl-pyrazole-4-sulfonamide (hydrochloride salt) (12 mg, 39%) ESI-MS m/z calc. 619.29407, found 620.2 (M+1)⁺; Retention time: 1.56 minutes; LC method A.

### Example 74: Preparation of Compound 251

### Step 1: 1-Bromo-2-((Z)-3-methylbut-1-enyl)benzene and 1-bromo-2-((E)-3-methylbut-1-enyl)benzene

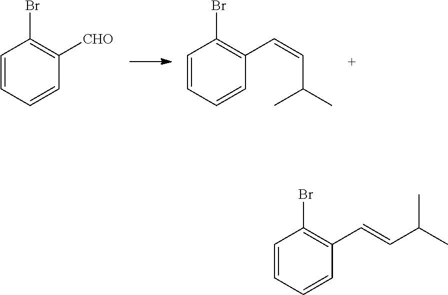

To a suspension of isobutyltriphenylphosphonium bromide (16.27 g, 40.75 mmol) in anhydrous tetrahydrofuran (56 mL) at 0° C. was slowly added 2.5M n-butyl lithium solution in hexane (17.6 mL, 44.14 mmol). The reaction mixture was stirred at ambient temperature for 2 hours. Then 2-bromobenzaldehyde (6.28 g, 33.96 mmol) in anhydrous tetrahydrofuran (22 mL) was added slowly and the resulting solution was stirred at ambient temperature for 16 hours. Saturated aqueous ammonium chloride solution (100 mL) was added and the organic layer was separated. The aqueous layer was extracted with hexanes (3×250 mL) and the combined organic layers were washed with brine (100 mL), dried over magnesium sulfate and concentrated. The residue was purified by silica gel column chromatography using hexanes to afford mixtures of 1-bromo-2-((Z)-3-methylbut-1-enyl)benzene and 1-bromo-2-((E)-3-methylbut-1-enyl)benzene (7.64 g, 100%) as a colorless oil. Mixtures of the two isomers were further purified by HPLC using 0-100% water-acetonitrile (0.1% trifluoroacetic acid). Pure fractions were combined, neutralized with saturated sodium bicarbonate aqueous solution and acetonitrile was evaporated off. The residue was extracted with hexanes. The organic extract was dried over magnesium sulfate and concentrated to afford 1-bromo-2-((Z)-3-methylbut-1-enyl)benzene (2.681 g, 35%) as a colorless oil and 1-bromo-2-((E)-3-methylbut-1-enyl)benzene (2.239 g, 29%) as a colorless oil.

1-Bromo-2-((Z)-3-methylbut-1-enyl)benzene. ¹H NMR (250 MHz, CDCl₃) δ (ppm): 7.58 (d, J=7.8 Hz, 1H), 7.27 (m, 2H), 7.10 (m, 1H), 6.31 (d, J=11.5 Hz, 1H), 5.58 (t, J=11.5 Hz, 1H), 2.65 (m, 1H), 1.02 (d, J=6.8 Hz, 6H).

1-Bromo-2-((E)-3-methylbut-1-enyl)benzene. ¹H NMR (250 MHz, CDCl₃) δ (ppm): 7.51 (m, 2H), 7.28 (m, 1H), 7.07 (m, 1H), 6.68 (d, J=15.8 Hz, 1H), 6.14 (dd, J=7.0, 15.8 Hz, 1H), 2.52 (m, 1H), 1.12 (d, J=6.8 Hz, 1H).

### Step 2: 1-bromo-2-(cis-2-isopropylcyclopropyl)benzene

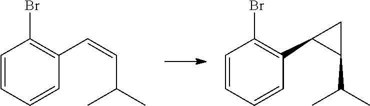

To anhydrous dichloromethane (24 mL) at 0° C. was added 1.0M diethylzine in hexane (23.7 mL, 23.7 mmol) followed by addition of a solution of trifluoracetic acid (2.71 g, 23.7 mmol) in anhydrous dichloromethane (12 mL). The reaction solution was stirred for 30 minutes at 0° C. A solution of diiodomethane (6.36 g, 23.7 mmol) in anhydrous dichloromethane (12 mL) was added dropwise and the reaction solution stirred for another 30 minutes at 0° C. A solution of 1-bromo-2-((Z)-3-methylbut-1-enyl)benzene (2.67 g, 11.9 mmol) in anhydrous dichloromethane (12 mL) was added dropwise to the above solution at 0° C. and the resulting reaction mixture was stirred at ambient temperature for 19 hours. 1 M hydrochloric acid (30 mL) was added and the layers were separated. The aqueous layer was extracted with dichloromethane (4×100 mL). The combined organic layers were washed with brine, dried over magnesium sulfate and concentrated. The residue was purified by revers phase column chromatography using 0-100% water-acetonitrile (0.1% trifluoroacetic acid). Pure fractions were combined, neutralized with saturated aqueous sodium bicarbonate solution and concentrated to remove most of acetonitrile. The residue was extracted with diethyl ether (3×50 mL) and the combined organic layer was dried over magnesium sulfate and concentrated to afford 1-bromo-2-(cis-2-isopropylcyclopropyl)benzene (1.85 g, 65%) as a pale yellow liquid. ¹H NMR (250 MHz, CDCl₃) δ (ppm): 7.55 (dd, J=1.0, 7.5 Hz, 1H), 7.19 (dd, J=1.0, 7.5 Hz, 1H), 7.05 (m, 2H), 2.29 (m, 1H), 1.02-0.94 (m, 7H), 0.93-0.66 (m, 3H).

### Step 3: 2-(2-(cis-2-isopropylcyclopropyl)phenyl)-4,4,5,5-tetramehyl-1,3,2-dioxaborolane

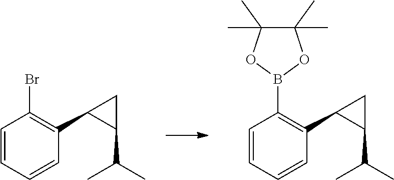 [SMILES: CC(C)[C@@H]1C[C@@H]1c1ccccc1Br ; CC(C)[C@@H]1C[C@@H]1c1ccccc1B1OC(C)(C)C(C)(C)O1]

1-Bromo-2-(cis-2-isopropylcyclopropyl)benzene (1.84 g, 7.69 mmol), Bis(pinacolato)diboron (2.15 g, 8.46 mmol), [1,1′-dis(diphenylphosphino)ferrocene]palladium(II) dichloride dichloromethane complex (314 mg, 0.38 mmol) and potassium acetate (2.26 g, 23.06 mmol) were dissolved in anhydrous 1,4-dioxane (30 mL). The reaction solution was purged with argon for 5 minutes and heated at 80° C. for 19 hours. The reaction solution cooled to ambient temperature and diluted with ether (500 mL), then washed with water, brine and dried over sodium sulfate, filtered and concentrated under the reduced pressure. The residue obtained was purified by silica gel chromatography using 0-15% hexane/dichloromethane to afford 2-(2-(cis-2-isopropylcyclopropyl)phenyl)-4,4,5,5-tetramehyl-1,3,2-dioxaborolane (1.40 g, 64%) as a pale yellow liquid. ¹H NMR (250 MHz, DMSO) δ (ppm): 7.62 (d, J=7.3 Hz, 1H), 7.34 (t, J=7.5 Hz, 1H), 7.15 (t, J=7.5 Hz, 1H), 7.05 (t, J=8.0 Hz, 1H), 2.70 (m, 1H), 1.30 (s, 12H), 0.91-0.82 (m, 7H), 0.8-0.5 (m, 3H).

### Step 4: N-[4-[2-[(1S,2S)-2-isopropylcyclopropyl]phenyl]-6-[4-(4-methylpiperazin-1-yl)phenoxy]pyrimidin-2-yl]-1-methyl-pyrazole-4-sulfonamide (Compound 251)

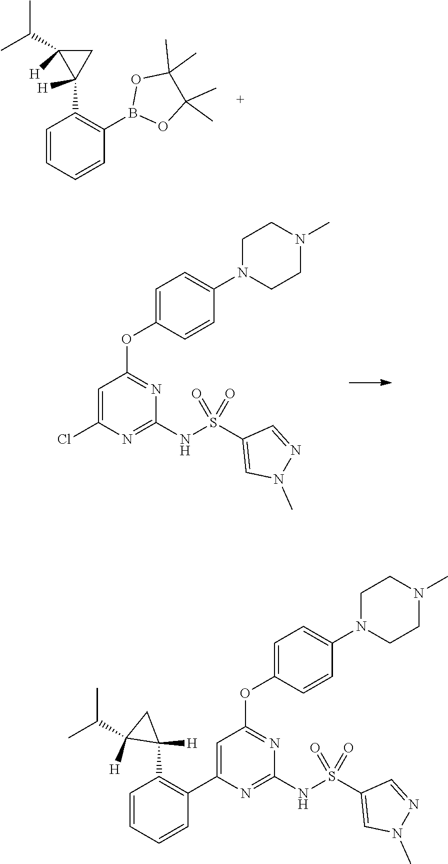 [SMILES: CC(C)[C@@H]1C[C@@H]1c1ccccc1B1OC(C)(C)C(C)(C)O1 ; CN1CCN(c2ccc(Oc3cc(Cl)nc(NS(=O)(=O)c4cnn(C)c4)n3)cc2)CC1 ; CC(C)[C@@H]1C[C@@H]1c1ccccc1-c1cc(Oc2ccc(N3CCN(C)CC3)cc2)nc(NS(=O)(=O)c2cnn(C)c2)n1]

A mixture of N-[4-chloro-6-[4-(4-methylpiperazin-1-yl)phenoxy]pyrimidin-2-yl]-1 -methyl-pyrazole-4-sulfonamide (formic acid salt) (25 mg, 0.04902 mmol), 2-[2-[(1S,2S)-2-isopropylcyclopropyl]phenyl]-4,4,5,5-tetramethyl-1,3,2-dioxaborolane (approximately 21.05 mg, 0.07353 mmol), K₂CO₃ (approximately 73.55 μL of 2 M, 0.1471 mmol) and Pd(PPh₃)₄ (approximately 11.33 mg, 0.009804 mmol) in dioxane (0.4 mL) was stirred at 100° C. for 16 hours. The reaction mixture was diluted with MeOH, filtered and purification by HPLC (1-99% ACN in water (HCl modifier)) gave N-[4-[2-[(1S,2S)-2-isopropylcyclopropyl]phenyl]-6-[4-(4-methylpiperazin-1-yl)phenoxy]pyrimidin-2-yl]-1-methyl-pyrazole-4-sulfonamide (hydrochloride salt) (20.8 mg, 68%). ESI-MS m/z calc. 587.2679, found 588.5 (M+1)⁺; Retention time: 1.39 minutes; LC method A.

### Example 75: Preparation of Compound 252

### Step 1: 1-Bromo-2-(3-methyl-butyl)-benzene

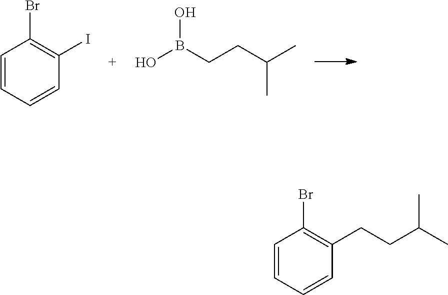

A mixture of 1-bromo-2-iodobenzene (11.0 g, 38.87 mmol), 3-methylbutylboronic acid (4.96 g, 42.76 mmol), potassium phosphate (16.50 g, 77.74 mmol) and 1,1′-bis(diphenylphosphino)ferrocene-palladium(II)dichloride dichloromethane complex (1.59 g, 1.94 mmol) in anhydrous tetrahydrofuran (155 mL) was bubbled with argon for 10 minutes, then sealed and stirred at 90° C. for 19 hours. Water (200 mL) and diethyl ether (300 mL) were added. The solution was filtered, and the organic layer was separated. The aqueous layer was extracted with ether (2×300 mL) and the combined organic layer was washed with brine (2×100 mL), dried over magnesium sulfate and concentrated. The residue was purified by silica gel column chromatography using hexane to afford the crude prude product, which was further purified by reverse phase column chromatography using 0-100% acetonitrile-water (0.1% trifluoroacetic acid). The pure fractions were combined and concentrated under the reduced pressure to remove most of acetonitrile, then extracted with diethyl ether (3×100 mL ). The combined organics was dried over magnesium sulfate and concentrated to afford 1-bromo-2-(3-methyl-butyl)-benzene (4.12 g, 47%) as a colorless oil. ¹H NMR (250 MHz, CDCl₃) δ (ppm): 7.53 (d, J=8.0 Hz, 1H), 7.23 (m, 2H), 7.05 (m, 1H), 2.74 (m, 2H), 1.64 (m, 1H), 1.51 (m, 2H), 0.98 (d, J=6.5 Hz, 6H).

### Step 2: 2-(2-Isopentylphenyl)-4,4,5,5-tetramethyl-1,3,2-dioxaborolane

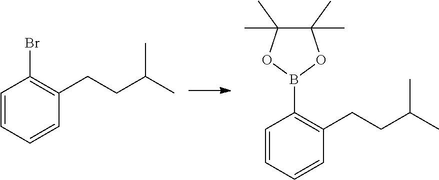 [SMILES: CC(C)CCc1ccccc1Br ; CC(C)CCc1ccccc1B1OC(C)(C)C(C)(C)O1]

Argon gas was bubbled through a mixture of 1-bromo-2-(3-methyl-butyl)-benzene (4.11 g, 18.10 mmol), 4,4,5,5-tetramethyl-2-(4,4,5,5-tetramethyl-1,3,2-dioxaborolan-2-yl)-1,3,2-dioxaborolane (5.05 g, 19.91 mmol), potassium acetate (5.33 g, 54.30 mmol) and 1,1′-bis(diphenylphosphino)ferrocene-palladium(II)dichloride dichloromethane complex (739 mg, 0.91 mmol) in anhydrous dioxane (90 mL) for 10 minutes. The reaction vessel was sealed, and the reaction mixture was stirred at 80° C. for 20 hours. Diethyl ether (200 mL) and water (100 mL) were added. The organic layer was separated, and the aqueous layer was extracted with diethyl ether (2×200 mL). The combined organic layer was washed with brine (2×50 mL), dried over magnesium sulfate and concentrated. The residue obtained was purified by silica gel column chromatography using 0-10% hexanes-dichloromethane to afford 2-(2-isopentylphenyl)-4,4,5,5-tetramethyl-1,3,2-dioxaborolane (3.22 g, 65%) as a pale yellow liquid. ¹H NMR (250 MHz, DMSO) δ (ppm): 7.61 (m, 1H), 7.37 (m, 1H), 7.15 (m. 2H), 2.80 (m, 2H), 1.58 (m, 1H), 1.36 (m, 2H), 1.34 (s, 12H), 0.92 (d, J=6.5 Hz, 6H).

### Step 3: N-[4-(2-isopentylphenyl)-6-[4-(4-methylpiperazin-1-yl)phenoxy]pyrimidin-2-yl]-1-methyl-pyrazole-4-sulfonamide (Compound 252)

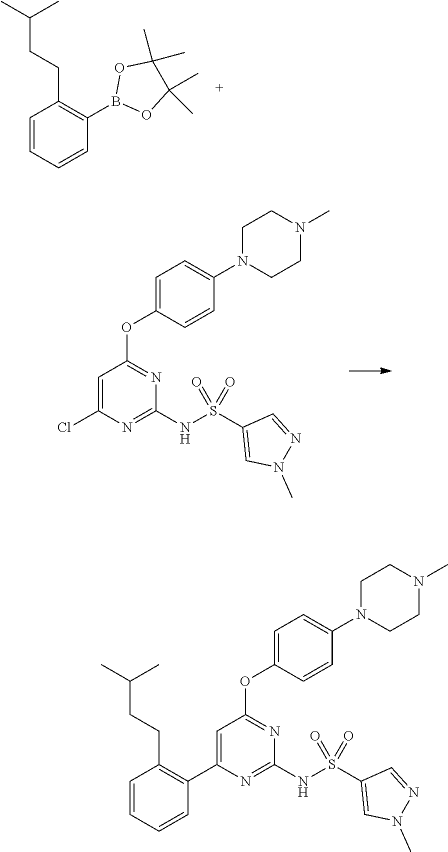 [SMILES: CC(C)CCc1ccccc1B1OC(C)(C)C(C)(C)O1 ; CN1CCN(c2ccc(Oc3cc(Cl)nc(NS(=O)(=O)c4cnn(C)c4)n3)cc2)CC1 ; CC(C)CCc1ccccc1-c1cc(Oc2ccc(N3CCN(C)CC3)cc2)nc(NS(=O)(=O)c2cnn(C)c2)n1]

A dioxane (0.5 mL) solution of 2-(2-isopentylphenyl)-4,4,5,5-tetramethyl-1,3,2-dioxaborolane (approximately 16.13 mg, 0.05883 mmol), N-[4-chloro-6-[4-(4-methylpiperazin-1-yl)phenoxy]pyrimidin-2-yl]-1-methyl-pyrazole-4-sulfonamide (formic acid salt) (20 mg, 0.03922 mmol), Pd(PPh₃)₄ (approximately 9.064 mg, 0.007844 mmol), and K₂CO₃ (approximately 78.45 μL of 2 M, 0.1569 mmol) was sparged with nitrogen for 30 seconds and then heated at 120° C. for 20 minutes. The mixture was diluted with MeOH (0.5 mL) and the solution was filtered and the filtrate purified by reverse phase chromatography using a 15 min gradient of 20% MeCN in water to 80% MeCN (HCl modifier) to give N-[4-(2-isopentylphenyl)-6-[4-(4-methylpiperazin-1-yl)phenoxy]pyrimidin-2-yl]-1-methyl-pyrazole-4-sulfonamide (hydrochloride salt) (6.8 mg, 28%). ¹H NMR (400 MHz, DMSO-d₆) δ 10.59 (s, 1H), 7.73 (s, 1H), 7.44-7.35 (m, 1H), 7.35-7.24 (m, 4H), 7.23-7.16 (m, 2H), 7.16-7.09 (m, 2H), 6.61 (s, 1H), 3.91-3.80 (m, 2H), 3.76 (s, 3H), 3.57-3.44 (m, 2H), 3.26-3.00 (m, 4H), 2.82 (d, J=4.7 Hz, 3H), 2.75-2.62 (m, 2H), 1.47-1.31 (m, 1H), 1.31-1.16 (m, 2H), 0.74 (d, J=6.6 Hz, 6H). ESI-MS m/z calc. 575.2679, found 576.4 (M+1)⁺; Retention time: 1.45 minutes; LC method A.

### Example 76: Preparation of Compound 253

### Step 1: 1-bromo-2-(isopropoxymethyl)benzene

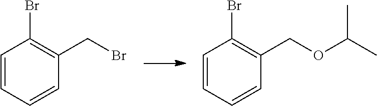

To a solution of isopopanol (3.60 g, 60 mmol) in anhydrous tetrahydrofuran (140 mL) at 0° C. was added a 60% suspension sodium hydride in mineral oil (2.4 g, 60 mmol) and the mixture was stirred at this temperature for 30 minutes. 1-Bromo-2-(bromomethyl)benzene (10 g, 40 mmol) was added dropwise. The reaction mixture was slowly warmed to ambient temperature and stirred for 48 hours. Diethyl ether (800 mL) and water (100 mL) were added. The organic layer was separated, washed with brine (2×100 mL), dried over magnesium sulfate and concentrated. The residue was purified by silica gel column chromatography using 0-20% hexanes-dichloromethane to afford 1-bromo-2-(isopropoxymethyl)benzene (8.32 g, 90%) as a colorless oil. ¹H NMR (250 MHz, CDCl₃) δ (ppm): 7.53 (m, 2H), 7.31 (m, 1H), 7.16 (m, 1H), 4.57 (s, 2H), 3.75 (m, 1H), 1.26 (d, J=6.0 Hz, 6H).

### Step 2: 2-(2-(isopropoxymethyl)phenyl)-4,4,5,5-tetramethyl-1,3,2-dioxaborolane

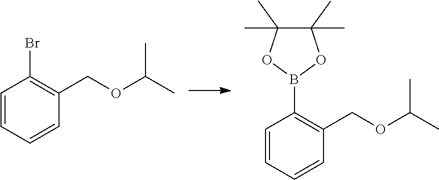 [SMILES: CC(C)OCc1ccccc1Br ; CC(C)OCc1ccccc1B1OC(C)(C)C(C)(C)O1]

Argon gas was bubbled through a mixture of 1-bromo-2-(isopropoxymethyl)benzene (8.33 g, 36.35 mmol), 4,4,5,5-tetramethyl-2-(4,4,5,5-tetramethyl-1,3,2-dioxaborolan-2-yl)-1,3,2-dioxaborolane (10.15 g, 39.99 mmol), potassium acetate (10.7 g, 0.109 mol) and 1,1′-bis(diphenylphosphino)ferrocene-palladium(II)dichloride dichloromethane complex (1.48 g, 1.82 mmol) in anhydrous dioxane (181 mL) for 10 minutes. The reaction vessel was sealed and the reaction mixture was stirred at 80° C. for 19 hours. Diethyl ether (900 mL) and water (100 mL) were added. The organic layer was separated, washed with brine (2×50 mL), dried over magnesium sulfate and concentrated. The residue was purified by silica gel column chromatography using 0-5% hexanes-diethyl ether to afford crude 5.1 g of crude material, contaminated with the ligand. The crude was purified by reverse phase column chromatography using 0-100% water-acetonitrile (0.1% trifluoroacetic acid). Pure fractions were combined, neutralized with saturated aqueous sodium bicarbonate solution and concentrated under the reduced pressure to remove most of the acetonitrile. The aqueous residue was extracted with diethyl ether (3×50 mL). The organic layer was dried over magnesium sulfate and concentrated to afford 2-(2-(isopropoxymethyl)phenyl)-4,4,5,5-tetramethyl-1,3,2-dioxaborolane (3.16 g, 33%) as a pale yellow liquid. ¹H NMR (250 MHz, DMSO) δ (ppm): 7.63 (m, 1H), 7.44 (m, 2H), 7.27 (m, 1H), 4.63 (s, 2H), 3.62 (m, 1H), 1.30 (s, 12H), 1.13 (d, J=6.0 Hz, 6H).

### Step 3: N-[4-[2-(isopropoxymethyl)phenyl]-6-[4-(4-methylpiperazin-1-yl)phenoxy]pyrimidin-2-yl]-1-methyl-pyrazole-4-sulfonamide (Compound 253)

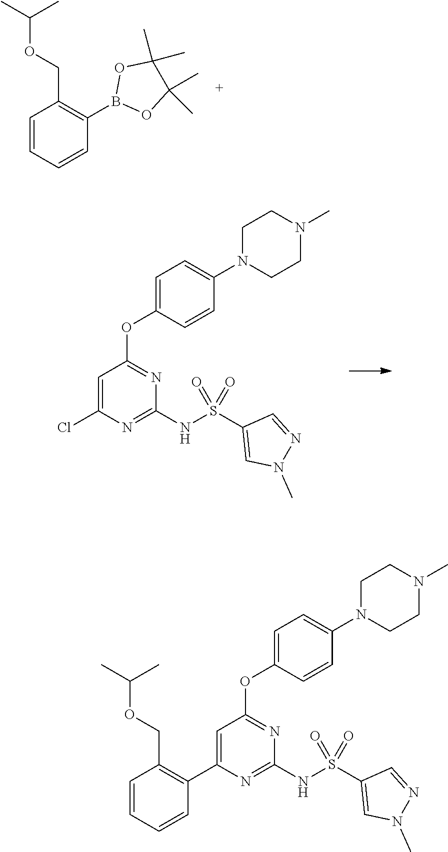 [SMILES: CC(C)OCc1ccccc1B1OC(C)(C)C(C)(C)O1 ; CN1CCN(c2ccc(Oc3cc(Cl)nc(NS(=O)(=O)c4cnn(C)c4)n3)cc2)CC1 ; CC(C)OCc1ccccc1-c1cc(Oc2ccc(N3CCN(C)CC3)cc2)nc(NS(=O)(=O)c2cnn(C)c2)n1]

A dioxane (0.5 mL) solution of 2-[2-(isopropoxymethyl)phenyl]-4,4,5,5-tetramethyl-1,3,2-dioxaborolane (approximately 16.25 mg, 0.05883 mmol), N-[4-chloro-6-[4-(4-methylpiperazin-1-yl)phenoxy]pyrimidin-2-yl]-1-methyl-pyrazole-4-sulfonamide (formic acid salt) (20 mg, 0.03922 mmol), Pd(PPh₃)₄ (approximately 9.064 mg, 0.007844 mmol), and K₂CO₃ (approximately 78.45 μL of 2 M, 0.1569 mmol) was sparged with nitrogen for 30 seconds and then heated at 120° C. for 20 minutes. The mixture was diluted with MeOH (0.5 mL) and the solution was filtered and the filtrate purified by reverse phase chromatography using a 15 minute gradient of 20% MeCN in water to 80% MeCN (HCl modifier) to give N-[4-[2-(isopropoxymethyl)phenyl]-6-[4-(4-methylpiperazin-1-yl)phenoxy]pyrimidin-2-yl]-1-methyl-pyrazole-4-sulfonamide (hydrochloride salt). ESI-MS m/z calc. 577.24713, found 578.0 (M+1)⁺; Retention time: 1.26 minutes; LC method A.

### Example 77: Preparation of Compound 254

### Step 1: 2-(3-isohexyloxyphenyl)-4,4,5,5-tetramethyl-1,3,2-dioxaborolane

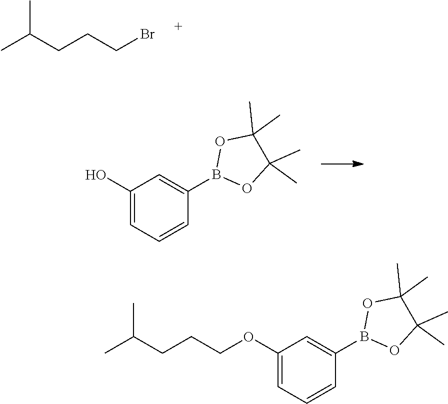

A DMF (1 mL) mixture of 3-(4,4,5,5-tetramethyl-1,3,2-dioxaborolan-2-yl)phenol (137.0 mg, 0.6225 mmol), 1-bromo-4-methyl-pentane (90 μL, 0.6183 mmol), and Cs₂CO₃ (600.2 mg, 1.842 mmol) was heated to 70° C. for 2 hours and then diluted with water (5 mL) and ethyl acetate (15 ml). The organic layer was washed with water (5 mL×3) and then brine (5 mL×2) and then dried with anhydrous sodium sulfate, filtered, and concentrated in vacuo to give 2-(3-isohexyloxyphenyl)-4,4,5,5-tetramethyl-1,3,2-dioxaborolane (134.2 mg, 71%) ESI-MS m/z calc. 304.22098, found 305.2 (M+1)⁺; Retention time: 0.92 minutes.

### Step 2: N-[4-(3-isohexyloxyphenyl)-6-[4-(4-methylpiperazin-1-yl)phenoxy]pyrimidin-2-yl]-1-methyl-pyrazole-4-sulfonamide (Compound 254)

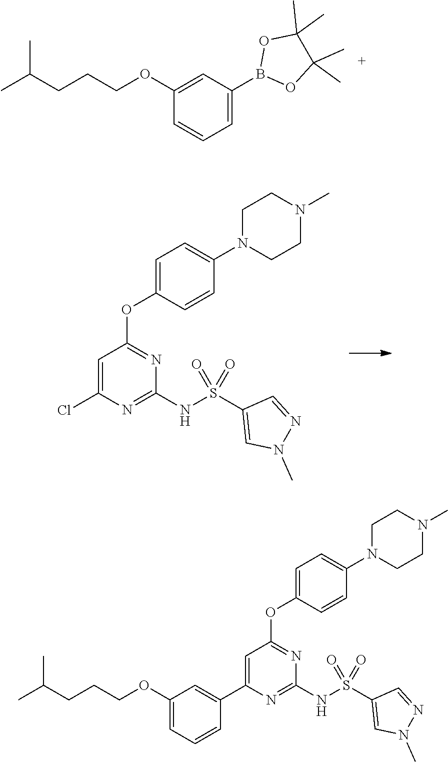

A dioxane (0.5 mL) solution of 2-(3-isohexyloxyphenyl)-4,4,5,5-tetramethyl-1,3,2-dioxaborolane (19.0 mg, 0.06245 mmol), N-[4-chloro-6-[4-(4-methylpiperazin-1-yl)phenoxy]pyrimidin-2-yl]-1-methyl-pyrazole-4-sulfonamide (formic acid salt) (20 mg, 0.03922 mmol), Pd(PPh₃)₄ (approximately 9.064 mg, 0.007844 mmol), and K₂CO₃ (approximately 78.45 μL of 2 M, 0.1569 mmol) was sparged with nitrogen for 30 seconds and then heated at 120° C. for 20 minutes. The mixture was diluted with MeOH (0.5 mL) and the solution was filtered and the filtrate purified by reverse phase chromatography using a 15 min gradient of 20% MeCN in water to 80% MeCN (HCl modifier) to give N-[4-(3-isohexyloxyphenyl)-6-[4-(4-methylpiperazin-1-yl)phenoxy]pyrimidin-2-yl]-1-methyl-pyrazole-4-sulfonamide (hydrochloride salt) (6.0 mg, 24%). ESI-MS m/z calc. 605.27844, found 606.47 (M+1)⁺; Retention time: 1.67 minutes; LC method A.

### Example 78: Preparation of Compound 255

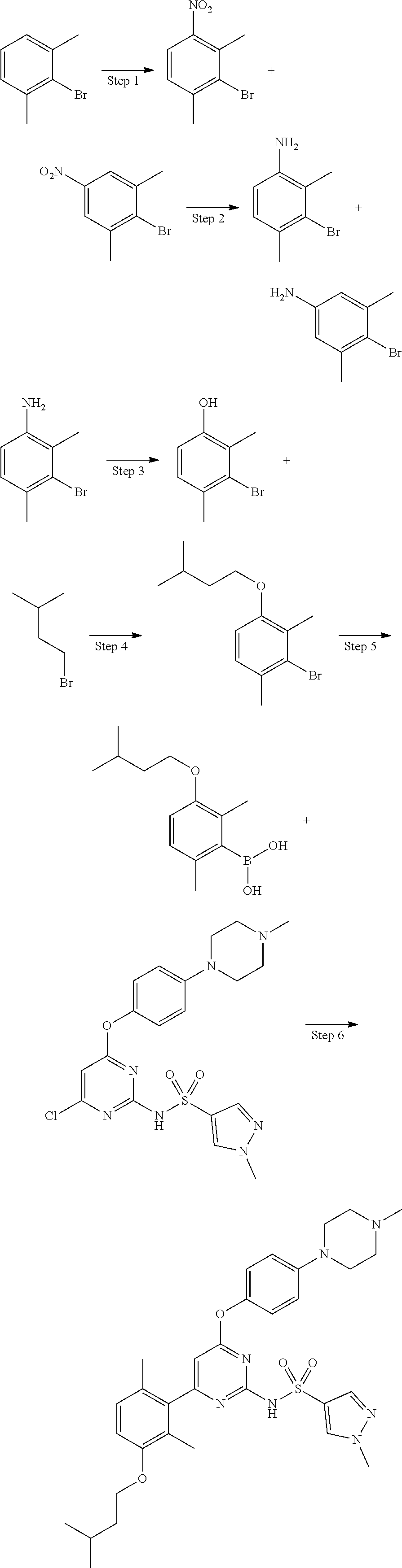

### Step 1: 2-Bromo-1,3-dimethyl-4-nitrobenzene and 2-bromo-1,3-dimethyl-5-nitrobenzene

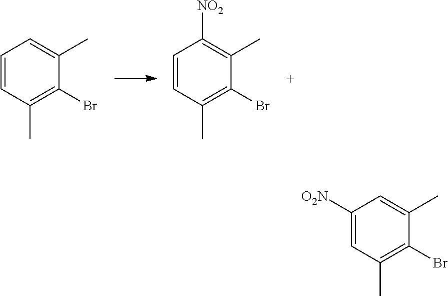

To a solution of 2,6-dimethyl-bromobenzene (95 g, 513 mmol) in glacial acetic acid (300 mL) was slowly added fuming nitric acid (150 mL) and the obtained reaction mixture was heated to 65° C. for 3 hours. After cooling to room temperature, the reaction mixture was poured into iced water (1200 mL). Precipitated yellow solid was isolated by filtration and dried to afford a crude product (85.2 g, 72%), containing an isomeric mixture of 2-bromo-1,3-dimethyl-4-nitrobenzene (major) and 2-bromo-1,3-dimethyl-5-nitrobenzene (minor) in approximately 6:1 ratio (by LCMS (UV254 nm)), that was used in the next step without further purification. 1H NMR (250 MHz, CDCl₃) δ (ppm): 7.93 (s, 0.3H), 7.65 (d, J=8.2 Hz, 1H), 7.22 (d, J=8.2 Hz, 1H), 2.59 (s, 3H), 2.52 (s, 1H), 2.50 (s, 3H).

### Step 2: 3-Bromo-2,4-dimethyl-phenylamine and 4-bromo-3,5-dimethyl-phenylamine

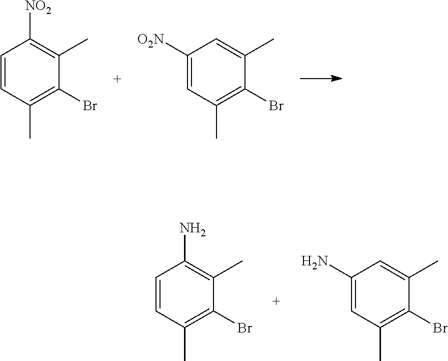

To a stirring solution of crude 2-bromo-1,3-dimethyl-4-nitrobenzene (85.16 g, 348 mmol) and diisopropylethylamine (224.9 g, 1.74 mol) in anhydrous acetonitrile (400 mL) at 0° C. was dropwise added a solution of trichlorosilane (122.1 mL, 1.21 mol) in anhydrous acetonitrile (100 mL). The mixture was allowed to warm up to room temperature and stirred for 16 hours. The reaction mixture was cooled to 0° C. and quenched with 2.0 M aqueous sodium hydroxide solution, filtered, volatiles removed under vacuum. The product was extracted with dichloromethane (3×200 mL). The insoluble part was filtered off and washed with dichloromethane (200 mL). The combined organic layers were washed with brine, dried over anhydrous sodium sulfate and concentrated. The obtained crude was purified by silica gel column chromatography using 0-15% hexanes-ethyl acetate to afford 3-bromo-2,4-dimethyl-phenylamine as a major, less polar isomer (48.41 g, 74%) and 4-bromo-3,5-dimethyl-phenylamine as minor, more polar isomer (7.33 g, 11%), both as brown solids. 3-Bromo-2,4-dimethyl-phenylamine: ¹H NMR (250 MHz, CDCl₃) δ (ppm): 6.91 (d, J=8.2 Hz, 1H), 6.57 (d, J=8.2 Hz, 1H), 3.59 (broad s, 2H), 2.33 (s, 3H), 2.31 (s, 3H). ESI-MS (m/z calc. 201.1, found 202.3 (M+1). Retention time: 2.60 minutes. 4-Bromo-3,5-dimethyl-phenylamine: ¹H NMR (250 MHz, CDCl₃) δ (ppm): 6.45 (s, 2H), 3.56 (broad s, 2H), 2.34 (s, 6H). ESI-MS (m/z calc. 201.1, found 202.3 (M+1). Retention time: 2.74 minutes.

### Step 3: 3-Bromo-2,4-dimethyl-phenol

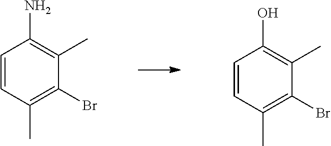

3-Bromo-2,4-dimethyl-phenylamine (48.41 g, 242 mmol) was dispersed in 1.0 M aqueous sulfuric acid (363 mL) and cooled to 0° C. Solution of sodium nitrite (25.05 g, 363 mmol) in water (55 mL) was added dropwise and the reaction mixture was stirred for 30 minutes at 0° C. 96.2% sulfuric acid (125 mL) was added slowly and the reaction mixture was heated to 100° C. for 1 hour. After cooling to room temperature, the reaction mixture was poured into iced water (1200 mL) and the product extracted with ethyl acetate (3×200 mL). The combined organic layers were washed with brine, dried over anhydrous sodium sulfate and concentrated. The obtained crude was purified by vacuum distillation (b.p. 92° C./2 torr) followed by recrystallization from hexanes at −20° C. to afford 3-bromo-2,4-dimethyl-phenol (17.96 g, 37%) as a yellow solid. ¹H NMR (250 MHz, CDCl₃) δ (ppm): 6.96 (d, J=8.0 Hz, 1H), 6.65 (d, J=8.0 Hz, 1H), 4.79 (s, 1H), 2.36 (s, 3H), 2.34 (s, 3H).

### Step 4: 2-Bromo-1,3-dimethyl-4-(3-methyl-butoxy)-benzene

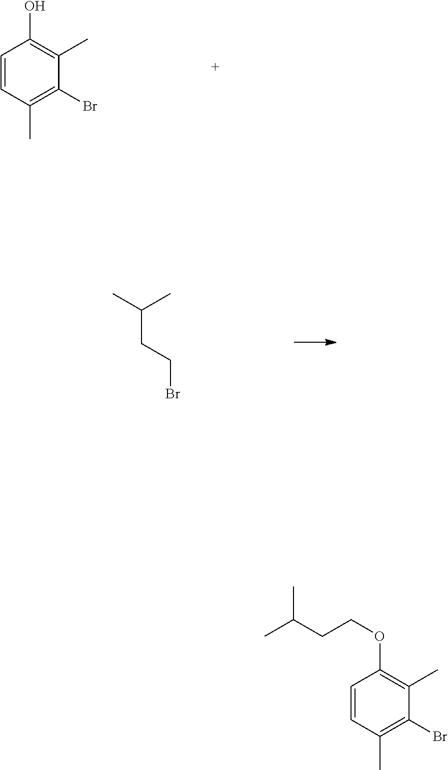

To a solution of crude 3-bromo-2,4-dimethyl-phenol (11.15 g) and 1-bromo-3-methylbutane (8.61 g, 57 mmol) in N-methyl-2-pyrrolidone (200 mL) was added anhydrous cesium carbonate (21.7 g, 66.5 mmol) and the reaction mixture was heated to 120° C. for 24 hours under nitrogen atmosphere. The reaction mixture was allowed to cool down to room temperature and then poured into a mixture of water (800 mL) and 1.0 M aqueous hydrochloric acid (200 mL). The product was extracted with ethyl acetate (3×200 mL), combined organic layers washed with brine, dried over anhydrous sodium sulfate and concentrated. The residue was purified by silica gel column chromatography using hexanes to afford 2-bromo-1,3-dimethyl-4-(3-methyl-butoxy)-benzene (6.02 g, 40%) as a colorless oil. ¹H NMR (250 MHz, CDCl₃) δ (ppm): 7.03 (d, J=8.0 Hz, 1H), 6.71 (d, J=8.0 Hz, 1H), 3.97 (t, J=6.0 Hz, 2H), 2.36 (s, 3H), 2.35 (s, 3H), 1.92-1.78 (m, 1H), 1.70 (q, J=6.0 Hz, 2H), 0.99 (s, 3H), 0.96 (s, 3H).

### Step 5: 2,6-Dimethyl-3-(3-methyl-butoxy)-phenyl boronic acid

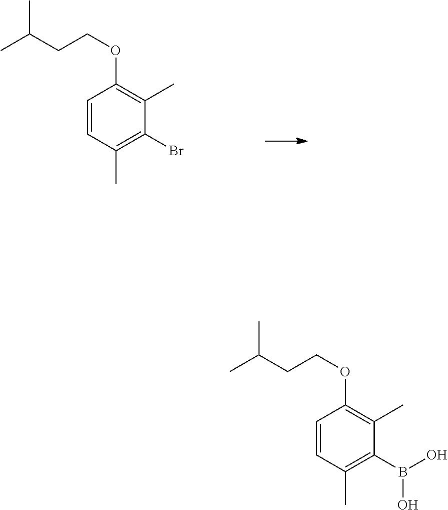 [SMILES: Cc1ccc(OCCC(C)C)c(C)c1Br ; Cc1ccc(OCCC(C)C)c(C)c1B(O)O]

To a solution of 2-bromo-1,3-dimethyl-4-(3-methyl-butoxy)-benzene (6.02 g, 22.2 mmol) in anhydrous tetrahydrofuran (35 mL) was dropwise added 2.26 M solution of n-butyllithium in hexanes (13.7 mL, 31 mmol) at −78° C. and the resulting mixture was stirred for 1 hour under dry nitrogen atmosphere. Trimethyl borate (3.22 g, 31 mmol) was added dropwise at −78° C. and the reaction mixture stirred for 1 hour at −78° C. and allowed to warm up to room temperature for overnight. After quenching with 1.0 M aqueous hydrochloric acid (50 mL), the product was extracted with ethyl acetate (3×100 mL). The combined organic layers washed with brine, dried over anhydrous sodium sulfate and concentrated. The residue was purified by silica gel column chromatography using 0-20% hexanes-acetone to afford 2,6-dimethyl-3-(3 -methyl-butoxy)-phenyl boronic acid (0.87 g, 17%) as a colorless oil. ¹H NMR (250 MHz, CDCl₃) δ (ppm): 6.97 (d, J=8.0 Hz, 1H), 6.74 (d, J=8.0 Hz, 1H), 4.57 (s, 2H), 3.96 (t, J=6.0 Hz, 2H), 2.32 (s, 3H), 2.25 (s, 3H), 1.90-1.84 (m, 1H), 1.71 (q, J=6.0 Hz, 2H), 0.98 (s, 3H), 0.95 (s, 3H). ESI-MS m/z calc. 236.1, found 237.2 (M+1). Retention time: 5.13 minutes

### Step 6: N-[4-(3-isopentyloxy-2,6-dimethyl-phenyl)-6-[4-(4-methylpiperazin-1-yl)phenoxy]pyrimidin-2-yl]-1-methyl-pyrazole-4-sulfonamide (Compound 255)

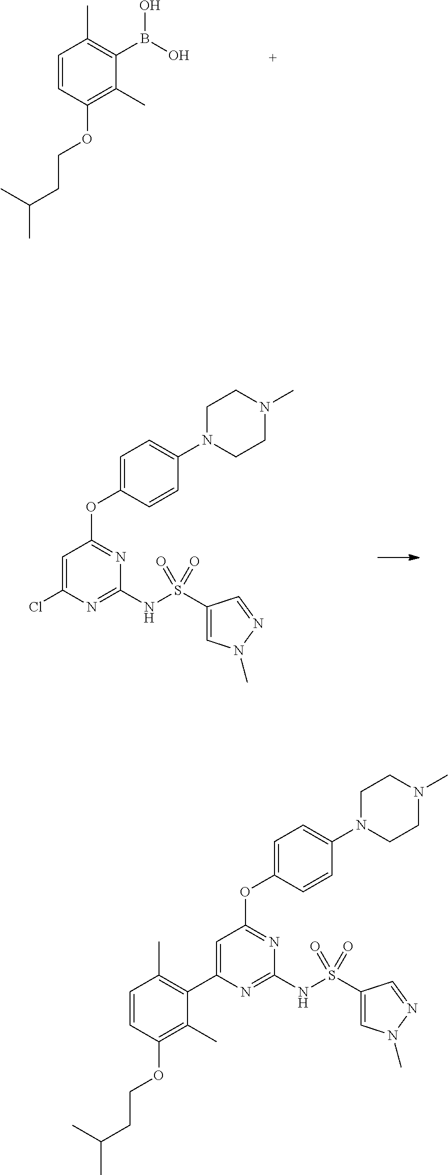 [SMILES: Cc1ccc(OCCC(C)C)c(C)c1B(O)O ; CN1CCN(c2ccc(Oc3cc(Cl)nc(NS(=O)(=O)c4cnn(C)c4)n3)cc2)CC1 ; Cc1ccc(OCCC(C)C)c(C)c1-c1cc(Oc2ccc(N3CCN(C)CC3)cc2)nc(NS(=O)(=O)c2cnn(C)c2)n1]

A dioxane (0.5 mL) solution of (3-isopentyloxy-2,6-dimethyl-phenyl)boronic acid (approximately 13.89 mg, 0.05883 mmol), N-[4-chloro-6-[4-(4-methylpiperazin-1-yl)phenoxy]pyrimidin-2-yl]-1-methyl-pyrazole-4-sulfonamide (formic acid salt) (20 mg, 0.03922 mmol), Pd(PPh₃)₄ (approximately 3.616 mg, 0.003129 mmol), and K₂CO₃ (approximately 39.10 μL of 2 M, 0.07820 mmol) was sparged with nitrogen for 30 seconds and then heated at 120° C. for 20 minutes. The mixture was diluted with MeOH (0.5 mL) and the solution was filtered and the filtrate purified by reverse phase chromatography using a 15 minute gradient of 20% MeCN in water to 80% MeCN (HCl modifier) to give N-[4-(3-isopentyloxy-2,6-dimethyl-phenyl)-6-[4-(4-methylpiperazin-1-yl)phenoxy]pyrimidin-2-yl]-1-methyl-pyrazole-4-sulfonamide (hydrochloride salt) (6.1 mg, 24%). ESI-MS m/z calc. 619.29407, found 620.55 (M+1)⁺; Retention time: 1.57 minutes; LC method A.

### Example 79: Preparation of Compound 256, Compound 257, Compound 258 and Compound 259

### Step 1: N-[4-chloro-6-(4-piperazin-1-ylphenoxy)pyrimidin-2-yl]-1-methyl-pyrazole-4-sulfonamide

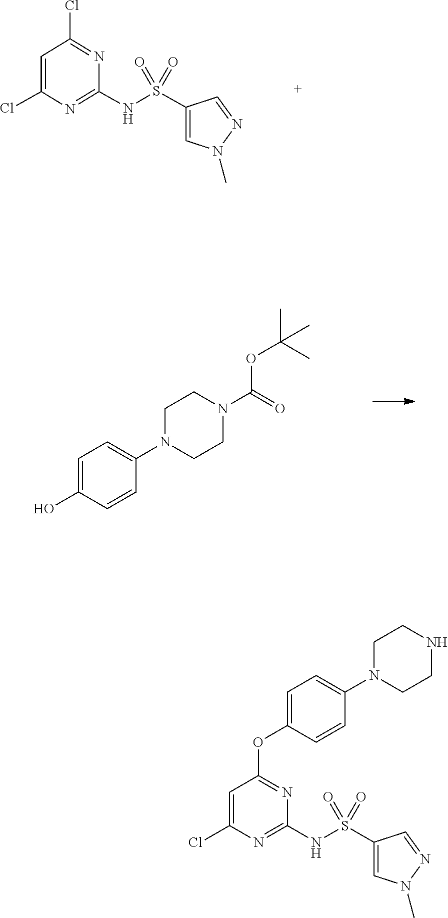 [SMILES: Cn1cc(S(=O)(=O)Nc2nc(Cl)cc(Cl)n2)cn1 ; CC(C)(C)OC(=O)N1CCN(c2ccc(O)cc2)CC1 ; Cn1cc(S(=O)(=O)Nc2nc(Cl)cc(Oc3ccc(N4CCNCC4)cc3)n2)cn1]

Stage 1: A heterogeneous solution of N-(4,6-dichloropyrimidin-2-yl)-1-methyl-pyrazole-4-sulfonamide (3 g, 9.736 mmol), tert-butyl 4-(4-hydroxyphenyl)piperazine-1-carboxylate (approximately 2.710 g, 9.737 mmol) and Potassium carbonate (approximately 4.037 g, 29.21 mmol) in NMP (60.81 mL) was heated in a sealed to 110° C. for 16 hours. Cesium carbonate (approximately 3.173 g, 9.737 mmol) was added and the reaction was further heated to 140° C. for 16 hours. The reaction mixture was cooled, diluted with water, and acetic acid (approximately 4.678 g, 4.430 mL, 77.90 mmol) was added. The aqueous solution was partitioned with ethyl acetate/hexanes (1:1). The organic layer was removed, and the aqueous layer was further extracted with ethyl acetate/hexanes (1:1, 4×). The combined organic extracts were dried using brine, magnesium sulfate, filtered and concentrated in vacuo. The crude residue was separated by flash column chromatography on silica gel (60% ethyl acetate in hexanes). The Boc-protected intermediate was isolated as a white foam.

Stage 2: The intermediate from stage one was dissolved in dioxane (20.0 mL) and hydrochloric acid (approximately 14.60 mL of 4 M, 58.42 mmol) in dioxane (4M) was slowly added. The reaction mixture was stirred for 140 minutes before removing the solvent in vacuo. N-[4-chloro-6-(4-piperazin-1-ylphenoxy)pyrimidin-2-yl]-1-methyl-pyrazole-4-sulfonamide was isolated as a white solid and used without further purification. ESI-MS m/z calc. 449.1037, found 450.43 (M+1)⁺; Retention time: 0.4 minutes;

### Step 2: 1-Methyl-N-[4-(o-tolyl)-6-(4-piperazin-1-ylphenoxy)pyrimidin-2-yl]pyrazole-4-sulfonamide (Compound 256)

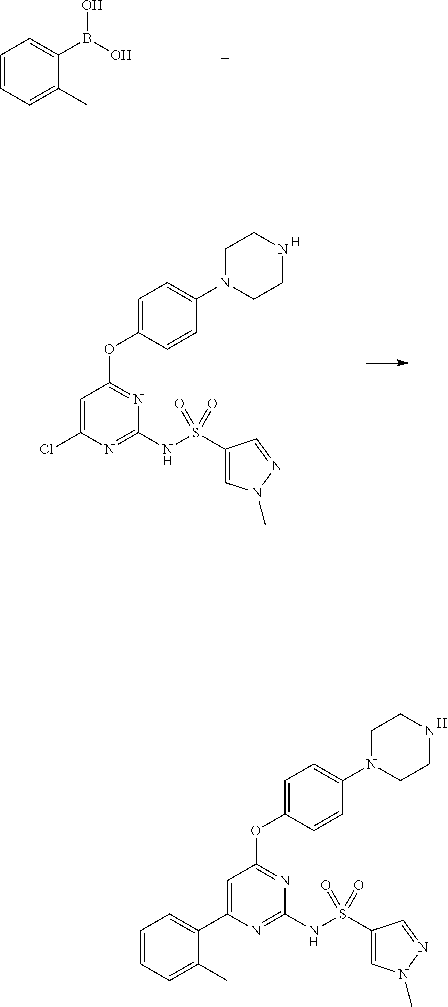

A heterogeneous mixture of o-tolylboronic acid (approximately 40.79 mg, 0.3000 mmol), N-[4-chloro-6-(4-piperazin-1-ylphenoxy)pyrimidin-2-yl]-1-methyl-pyrazole-4-sulfonamide (approximately 44.99 mg, 0.1000 mmol), potassium carbonate (approximately 23.04 mg, 0.1667 mmol), and tetrakis(triphenylphosphine)palladium(0) (approximately 7.704 mg, 0.006667 mmol) in dioxane (500.0 μL) (0.50 mL) and water (0.05 mL) was microwaved at 120° C. in a sealed vial for 15 minutes. The reaction vials were diluted with DMSO (0.50 mL) and acidified using hydrochloric acid (98.54 μL of 37% w/v, 1.000 mmol). The resultant crude mixture was separated by reverse phase HPLC (acetonitrile in water with 0.1% hydrochloric acid) to give 1-methyl-N-[4-(o-tolyl)-6-(4-piperazin-1-ylphenoxy)pyrimidin-2-yl]pyrazole-4-sulfonamide (11.7 mg, 22%). ESI-MS m/z calc. 505.1896, found 506.49 (M+1)⁺; Retention time: 1.16 minutes; LC method A.

### Step 3: 1-Methyl-N-[4-(4-piperazin-1-ylphenoxy)-6-(2,4,6-trimethylphenyl)pyrimidin-2-yl]pyrazole-4-sulfonamide (Compound 257)

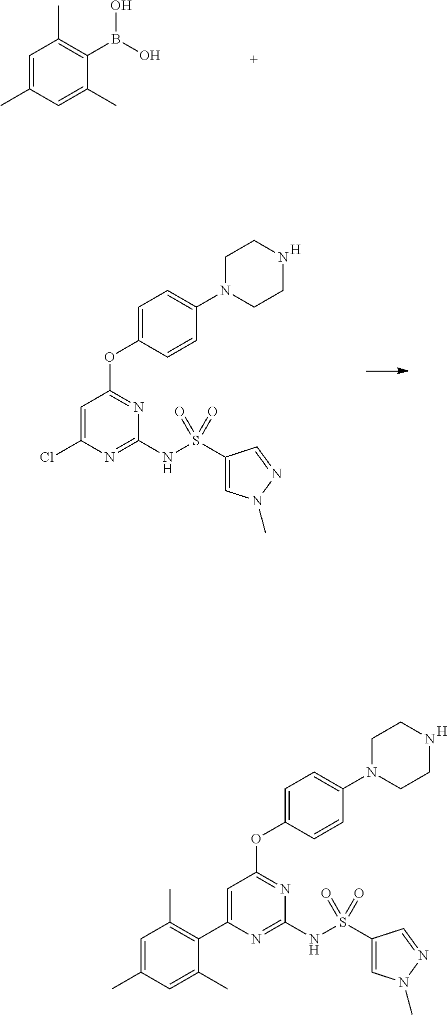

A heterogeneous mixture of (2,4,6-trimethylphenyl)boronic acid (approximately 49.20 mg, 0.3000 mmol), N-[4-chloro-6-(4-piperazin-1-ylphenoxy)pyrimidin-2-yl]-1-methyl-pyrazole-4-sulfonamide (approximately 44.99 mg, 0.1000 mmol), potassium carbonate (approximately 23.04 mg, 0.1667 mmol), and tetrakis(triphenylphosphine)palladium(0) (approximately 7.704 mg, 0.006667 mmol) in dioxane (500.0 μL) (0.50 mL) and water (0.05mL) was microwaved at 120° C. in a sealed vial for 15 minutes. The reaction vials were diluted with DMSO (0.50 mL) and acidified using hydrochloric acid (98.54 μL of 37% w/v, 1.000 mmol). The resultant crude mixture was separated by HPLC (acetonitrile in water with 0.1% hydrochloric acid) to afford 1-Methyl-N-[4-(4-piperazin-1-ylphenoxy)-6-(2,4,6-trimethylphenyl)pyrimidin-2-yl]pyrazole-4-sulfonamide (2.1 mg, 4%). ESI-MS m/z calc. 533.2209, found 534.54 (M+1)⁺; Retention time: 1.3 minutes; LC method A.

### Step 4: N-[4-(3-chloro-2-methyl-phenyl)-6-(4-piperazin-1-ylphenoxy)pyrimidin-2-yl]-1-methyl-pyrazole-4-sulfonamide (Compound 258)

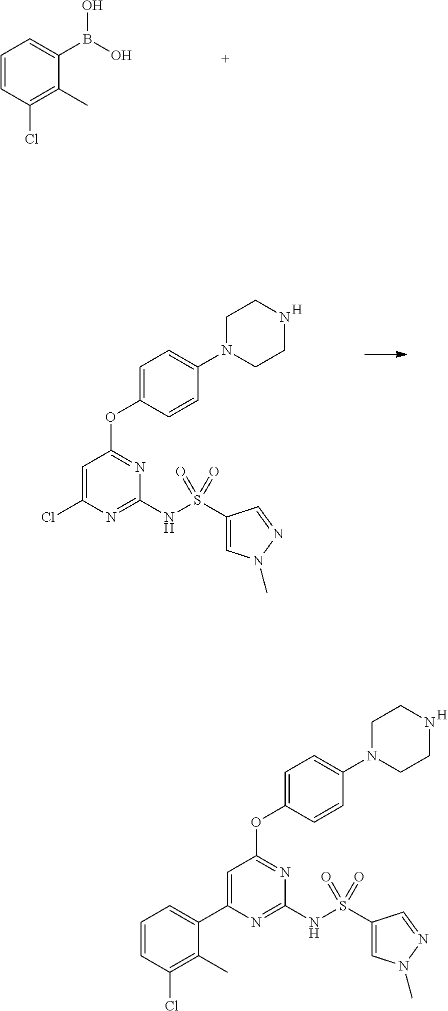

A heterogeneous mixture of (3-chloro-2-methyl-phenyl)boronic acid (approximately 51.12 mg, 0.3000 mmol), N-[4-chloro-6-(4-piperazin-1-ylphenoxy)pyrimidin-2-yl]-1-methyl-pyrazole-4-sulfonamide (approximately 44.99 mg, 0.1000 mmol), potassium carbonate (approximately 23.04 mg, 0.1667 mmol), and tetrakis(triphenylphosphine)palladium(0) (approximately 7.704 mg, 0.006667 mmol) in dioxane (500.0 μL) (0.50 mL) and water (0.05 mL) was microwaved at 120° C. in a sealed vial for 15 minutes. The reaction vials were diluted with DMSO (0.50 mL) and acidified using hydrochloric acid (98.54 μL of 37% w/v, 1.000 mmol). The resultant crude mixture was separated by HPLC (acetonitrile in water with 0.1% hydrochloric acid). N-[4-(3-chloro-2-methyl-phenyl)-6-(4-piperazin-1-ylphenoxy)pyrimidin-2-yl]-1-methyl-pyrazole-4-sulfonamide (11 mg, 19%). ESI-MS m/z calc. 539.15063, found 540.45 (M+1)⁺; Retention time: 1.34 minutes; LC method A.

### Step 5: N-[4-(2-cyclobutylphenyl)-6-(4-piperazin-1-ylphenoxy)pyrimidin-2-yl]-1 -methyl-pyrazole-4-sulfonamide (Compound 259)

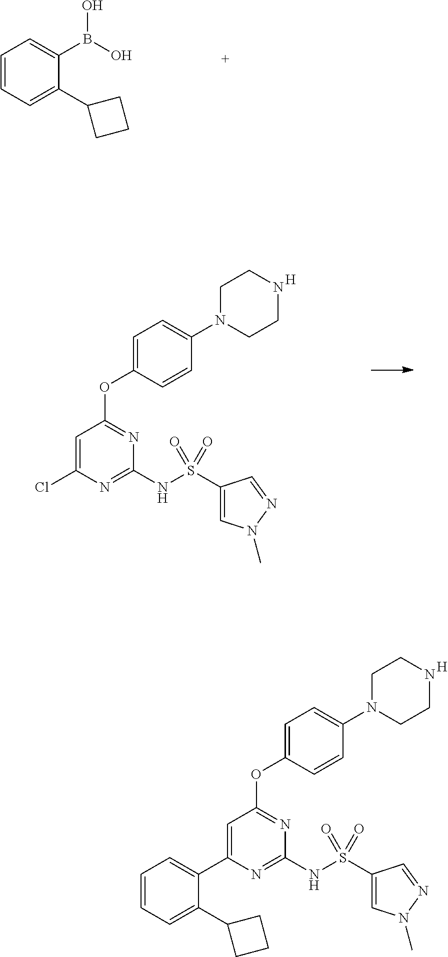

A heterogeneous mixture of (2-cyclobutylphenyl)boronic acid (approximately 52.81 mg, 0.3000 mmol), N-[4-chloro-6-(4-piperazin-1-ylphenoxy)pyrimidin-2-yl]-1-methyl-pyrazole-4-sulfonamide (approximately 44.99 mg, 0.1000 mmol), potassium carbonate (approximately 23.04 mg, 0.1667 mmol), and tetrakis(triphenylphosphine)palladium(0) (approximately 7.704 mg, 0.006667 mmol) in dioxane (500.0 μL) (0.50 mL) and water (0.05 mL) was microwaved at 120° C. in a sealed vial for 15 minutes. The reaction vials were diluted with DMSO (0.50 mL) and acidified using hydrochloric acid (98.54 μL of 37% w/v, 1.000 mmol). The resultant crude mixture was separated by HPLC (acetonitrile in water with 0.1% hydrochloric acid). N-[4-(2-cyclobutylphenyl)-6-(4-piperazin-1-ylphenoxy)pyrimidin-2-yl]-1-methyl-pyrazole-4-sulfonamide (13.3 mg, 23%). ESI-MS m/z calc. 545.2209, found 546.51 (M+1)⁺; Retention time: 1.38 minutes; LC method A.

### Example 80: Preparation of Compound 260

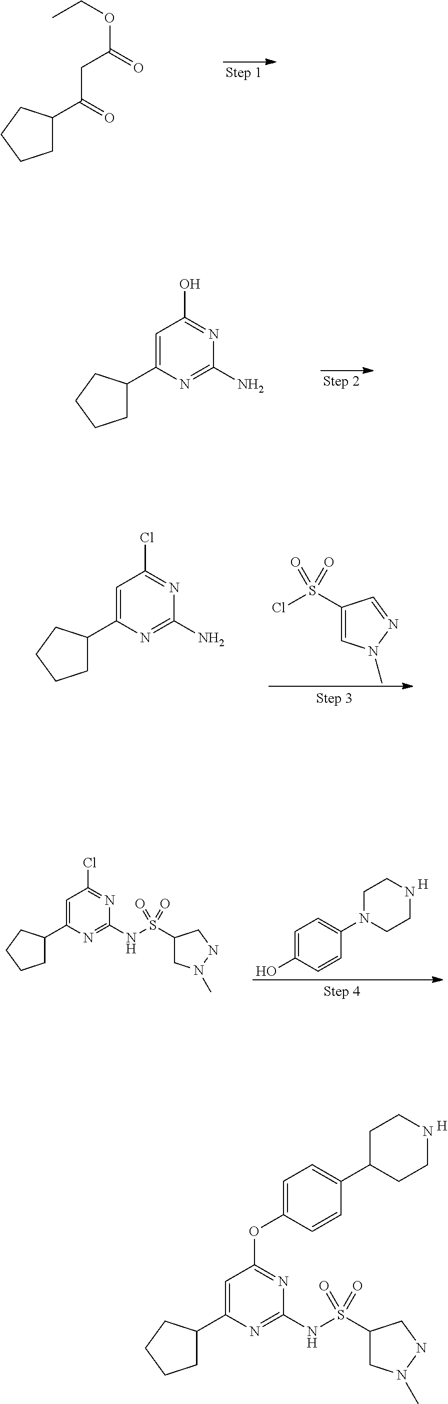

### Step 1: 2-amino-6-cyclopentyl-pyrimidin-4-ol

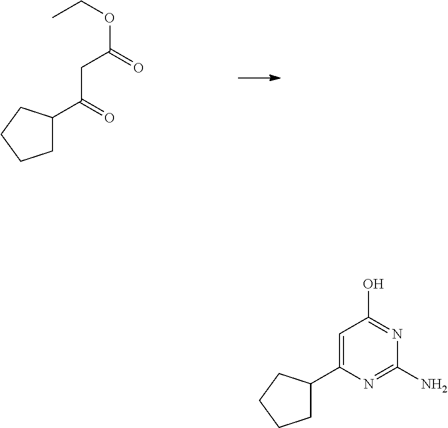

To a solution of ethyl 3-cyclopentyl-3-oxo-propanoate (4.07 g, 22.09 mmol) and guanidine (hydrochloride salt) (approximately 2.533 g, 26.51 mmol) in methanol (70 mL) at room temperature was added potassium tert-butoxide (13.3669 g, 119.1 mmol) portion wise over 30 minutes with vigorous stirring, and the reaction was warmed to 60° C. and stirred for 30 minutes. The reaction was then cooled to room temperature and stirred overnight. The precipitated salt was removed by filtration. The solution was concentrated to about 8 mL of methanol, water (8 mL) was added, cooled in an ice bath and the pH was adjusted to about 5 by adding 6.0 N HCl. The resulting precipitate was filtered, dried via suction and then under high vacuum. The yellow solid obtained (4.50 g) was triturated in MTBE (15 mL), filtered and dried to afford 2-amino-6-cyclopentyl-pyrimidin-4-ol (3.16 g, 80%)¹H NMR (400 MHz, DMSO-d₆) δ 10.72 (s, 1H), 6.54 (s, 2H), 5.40 (s, 1H), 2.65 (m, 1H), 1.80 (m, 2H), 1.60 (m, 6H). ESI-MS m z calc. 179.10587, found 180.22 (M+1)⁺; Retention time: 0.25 minutes; LC method D.

### Step 2: 4-Chloro-6-cyclopentyl-pyrimidin-2-amine

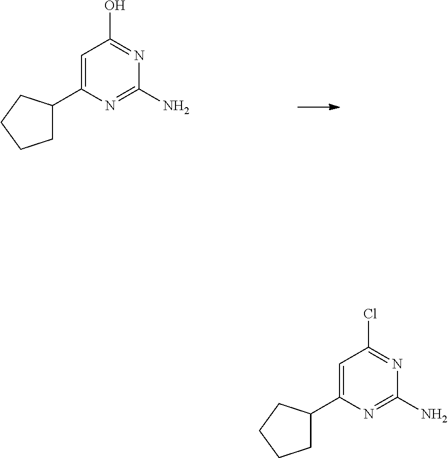 [SMILES: Nc1nc(O)cc(C2CCCC2)n1 ; Nc1nc(Cl)cc(C2CCCC2)n1]

2-Amino-6-cyclopentyl-pyrimidin-4-ol (1.50 g, 8.370 mmol) was added to phosphorus oxychloride (11.50 g, 75.00 mmol). The reaction vial was capped, and the suspension was allowed to stir at 105° C. for 1 hour. The volatiles were removed under reduced pressure and the remaining residue was diluted with DCM (5 mL) and cooled to 0° C. before being stirred with saturated aqueous sodium bicarbonate (75 mL) for 15 minutes. The pH was then raised from 5 to 8 with the addition of 1 N NaOH (approximately 10-15 mL). The mixture was then allowed to stir at room temperature for 2 hours. Afterwards, it was extracted with DCM (3×50 mL). The organic layers were combined, dried over sodium sulfate, filtered and concentrated under reduced pressure. The product was isolated by silica gel column chromatography eluting with a 0-30% EtOAc/hexane gradient over 25 minutes. 4-chloro-6-cyclopentyl-pyrimidin-2-amine (679 mg, 41%) was obtained as a white crystalline solid. ESI-MS m/z calc. 197.07198, found 198.3 (M+1)⁺; Retention time: 1.17 minutes; LC method A.

### Step 3: N-(4-chloro-6-cyclopentyl-pyrimidin-2-yl)-1-methyl-pyrazole-4-sulfonamide

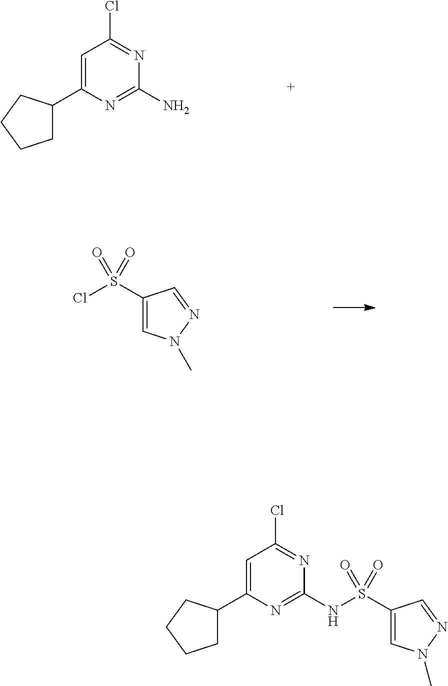

4-chloro-6-cyclopentyl-pyrimidin-2-amine (679 mg, 3.435 mmol) was dissolved in DMF (7 mL) and cooled to 0° C. before the addition of sodium hydride (321 mg, 13.38 mmol). The mixture was stirred at 0° C. for 5 minutes, then at room temperature for another 20 minutes. The mixture was again cooled to 0° C., and 1-methylpyrazole-4-sulfonyl chloride (1.53 g, 5.083mmol) was added in one portion. The reaction mixture was allowed to stir at 0° C. for 5 minutes and then at room temperature for 40 minutes. The reaction mixture was cooled to 0° C. and poured into ice water (50 mL). It was mixed with EtOAc (50 mL), and the layers were separated. The organic layer was further extracted with an aqueous solution of 0.5 M NaOH (2×50 mL). All aqueous layers were combined and acidified to pH 4 with the addition of 1 M HCl. The resulting white suspension was then extracted with EtOAc (3×50 mL). The final organic layers were combined, dried over sodium sulfate, filtered and concentrated under reduced pressure. The obtained orange-yellow oil was subjected to silica gel column chromatography eluting with a 0-50% EtOAc/hexane gradient. The pure fractions were combined and concentrated to provide N-(4-chloro-6-cyclopentyl-pyrimidin-2-yl)-1-methyl-pyrazole-4-sulfonamide (697 mg, 59%) as a clear colorless oil that crystallized upon standing to a white solid. ¹H NMR (400 MHz, DMSO-d₆) δ 11.89 (s, 1H), 8.37 (s, 1H), 7.86 (d, J=0.7 Hz, 1H), 7.16 (s, 1H), 3.87 (s, 3H), 3.06 (p, J=8.2 Hz, 1H), 1.92 (d, J=5.5 Hz, 2H), 1.82-1.69 (m, 2H), 1.69-1.55 (m, 4H). ESI-MS m/z calc. 341.07132, found 342.3 (M+1)⁺; Retention time: 1.4 minutes; LC method A.

### Step 4: N-[4-cyclopentyl-6-(4-piperazin-1-ylphenoxy)pyrimidin-2-yl]-1-methyl-pyrazole-4-sulfonamide (Compound 260)

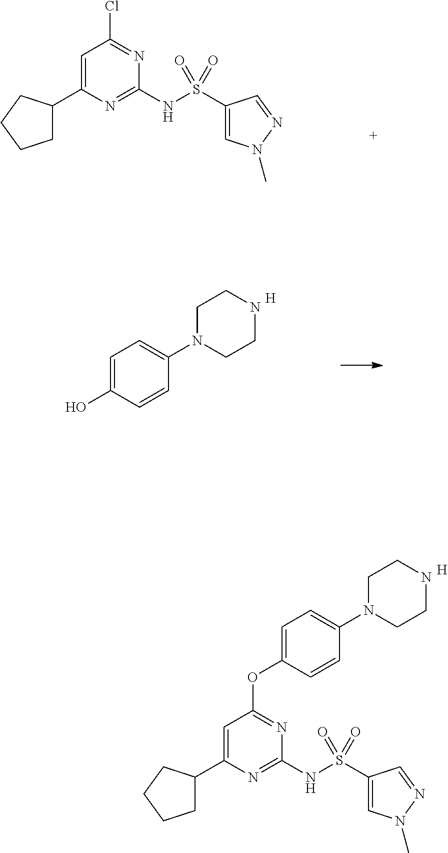

A solution of N-(4-chloro-6-cyclopentyl-pyrimidin-2-yl)-1-methyl-pyrazole-4-sulfonamide (25 mg, 0.07314 mmol) in NMP (400 μL) was added to 4-piperazin-1-ylphenol (approximately 52.15 mg, 0.2926 mmol). Cesium carbonate (120 mg, 0.3683 mmol) was added, and the reaction mixture was allowed to stir at 100° C. overnight. The reaction mixture was diluted with DMSO and purified by reverse-phase HPLC: Samples were purified using a reverse phase HPLC method using a Luna C₁₈ (2) column (50×21.2 mm, 5 μm particle size) sold by Phenomenex (pn: 00B-4252-PO-AX), and a dual gradient run from 10-70% mobile phase B over 15.0 minutes. Mobile phase A=water (5 mM HCl acid modifier). Mobile phase B=acetonitrile. Flow rate=35 mL/min, injection volume=950 μL, and column temperature=25° C. The UV trace at 220 nm was used to collect fractions, to give N-[4-cyclopentyl-6-(4-piperazin-1-ylphenoxy)pyrimidin-2-yl]-1-methyl-pyrazole-4-sulfonamide (16 mg, 42%). ESI-MS m/z calc. 483.20526, found 484.2 (M+1)⁺; Retention time: 0.85 minutes; LC method A.

### Example 81: Preparation of Compound 261

### Step 1: N-[4-(2-isobutylphenyl)-6-(4-piperazin-1-ylphenoxy)pyrimidin-2-yl]-1 -methyl-pyrazole-4-sulfonamide (Compound 261)

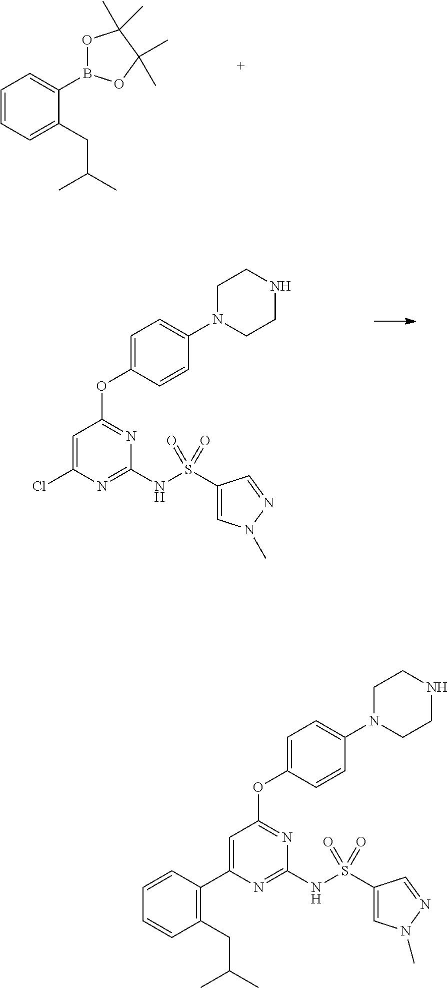 [SMILES: CC(C)Cc1ccccc1B1OC(C)(C)C(C)(C)O1 ; Cn1cc(S(=O)(=O)Nc2nc(Cl)cc(Oc3ccc(N4CCNCC4)cc3)n2)cn1 ; CC(C)Cc1ccccc1-c1cc(Oc2ccc(N3CCNCC3)cc2)nc(NS(=O)(=O)c2cnn(C)c2)n1]

A heterogeneous mixture of 2-(2-isobutylphenyl)-4,4,5,5-tetramethyl-1,3,2-dioxaborolane (78.05 mg, 0.3000 mmol), Tetrakis(triphenylphosphine)palladium(0) (11.6 mg, 0.0100 mmol), N-[4-chloro-6-(4-piperazin-1-ylphenoxy)pyrimidin-2-yl]-1-methyl-pyrazole-4-sulfonamide (45.0 mg, 0.100 mmol) and potassium carbonate (69.1 mg, 0.500 mmol) in dioxane (440 μL) and water (44 μL) was microwaved for 20 minutes at 120° C. in a sealed pressure tube. The reaction mixture was diluted with DMSO (0.5 mL) and hydrochloric acid (80 μL of 37 % w/v, 0.812 mmol) was added. The solution was filtered and then separated by HPLC (acetonitrile in water with 0.1% hydrochloric acid) to afford N-[4-(2-isobutylphenyl)-6-(4-piperazin-1-ylphenoxy)pyrimidin-2-yl]-1-methyl-pyrazole-4-sulfonamide (2.5 mg, 5%) as a white solid. ESI-MS m/z calc. 547.2366, found 548.61 (M+1)⁺; Retention time: 1.42 minutes; LC method A.

### Example 82: Preparation of Compound 262

### Step 1: N-[4-(2,6-diisopropylphenyl)-6-(4-piperazin-1-ylphenoxy)pyrimidin-2-yl]-1 -methyl-pyrazole-4-sulfonamide (Compound 262)

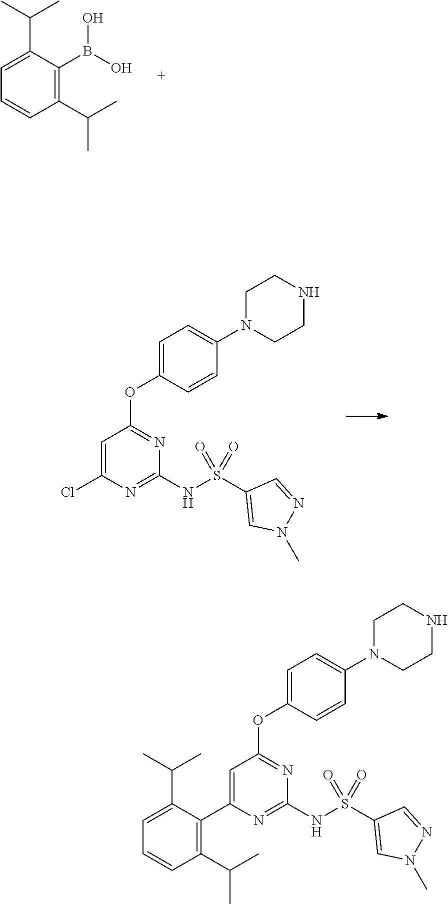 [SMILES: CC(C)c1cccc(C(C)C)c1B(O)O ; Cn1cc(S(=O)(=O)Nc2nc(Cl)cc(Oc3ccc(N4CCNCC4)cc3)n2)cn1 ; CC(C)c1cccc(C(C)C)c1-c1cc(Oc2ccc(N3CCNCC3)cc2)nc(NS(=O)(=O)c2cnn(C)c2)n1]

A heterogeneous mixture of N-[4-chloro-6-(4-piperazin-1-ylphenoxy)pyrimidin-2-yl]-1-methyl-pyrazole-4-sulfonamide (20.00 mg, 0.04445 mmol), (1,3-Bis(2,6-diisopropylphenyl)imidazolidene) (3-chloropyridyl) palladium(II) dichloride (3.0 mg, 0.0044 mmol), (2,6-diisopropylphenyl)boronic acid (27.5 mg, 0.1334 mmol), and potassium tert-butoxide (20.0 mg, 0.178 mmol) in tert-butanol (180 μL) was microwaved for 30 min at 65° C. in a sealed pressure tube. The reaction was diluted with DMSO (0.5 mL) and acidified with hydrochloric acid (35 μL of 37% w/v, 0.355 mmol). The crude solution was separated by HPLC (acetonitrile in water with 0.1% hydrochloric acid). N-[4-(2,6-diisopropylphenyl)-6-(4-piperazin-1-ylphenoxy)pyrimidin-2-yl]-1-methyl-pyrazole-4-sulfonamide (1.3 mg, 5%) was isolated as a white solid. ESI-MS m/z calc. 575.2679, found 576.62 (M+1)⁺; Retention time: 1.49 minutes; LC method A.

### Example 83: Preparation of Compound 263

### Step 1: N-[4-(1-cyano-1-methyl-ethyl)-6-phenoxy-pyrimidin-2-yl]-1-methyl-pyrazole-4-sulfonamide (Compound 263)

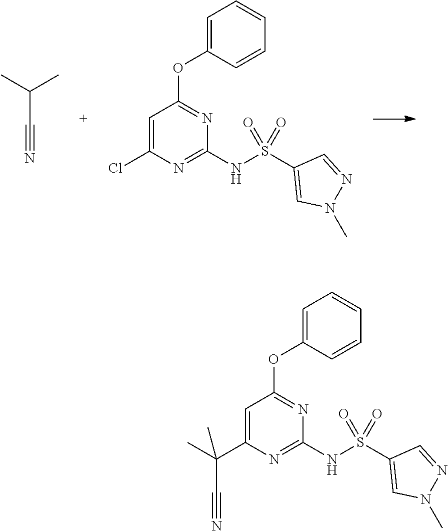

To a solution of isobutyronitrile (approximately 13.82 mg, 17.95 μL, 0.2000 mmol) in toluene (0.2 mL) was added LDA (approximately 100.0 μL of 2 M, 0.2000 mmol) followed by N-(4-chloro-6-phenoxy-pyrimidin-2-yl)-1-methyl-pyrazole-4-sulfonamide (36.58 mg, 0.1 mmol), Pd(OAc)₂ (approximately 2.245 mg, 0.01000 mmol) and 4,6,11-triisobutyl-1,4,6,11-tetraza-5-phosphabicyclo[3.3.3]undecane (approximately 6.850 mg, 7.106 μL, 0.02000 mmol). The reaction vial was sealed and heated to 100° C. for 1 hour. No conversion observed. Added [1,1′-Bis(diphenylphosphino)ferrocene]dichloropalladium(II) (approximately 7.317 mg, 0.01000 mmol) and continued stirring for 2 d at 100° C. The reaction was cooled and 0.020 mL of acetic acid was added. The solvent was evaporated in vacuo. The crude residue was dissolved in DMSO (1.0 mL) and separated by reverse phase chromatography (0 to 100% acetonitrile in water with 0.1% hydrochloric acid). N-[4-(1-cyano-1-methyl-ethyl)-6-phenoxy-pyrimidin-2-yl]-1-methyl-pyrazole-4-sulfonamide (2.7 mg, 7%) was isolated as a white solid. ESI-MS m/z calc. 398.11612, found 399.38 (M+1)⁺; Retention time: 1.39 minutes; LC method A.

### Example 84: Preparation of Compound 264

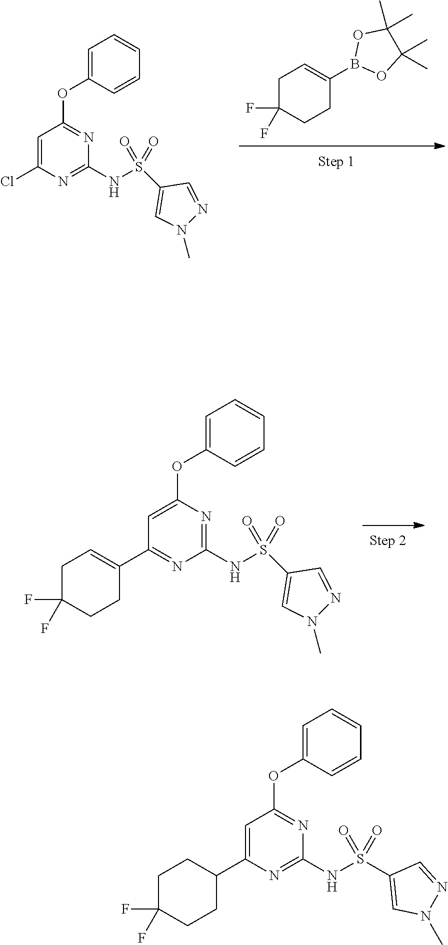 [SMILES: Cn1cc(S(=O)(=O)Nc2nc(Cl)cc(Oc3ccccc3)n2)cn1 ; CC1(C)OB(C2=CCC(F)(F)CC2)OC1(C)C ; Cn1cc(S(=O)(=O)Nc2nc(Oc3ccccc3)cc(C3=CCC(F)(F)CC3)n2)cn1 ; Cn1cc(S(=O)(=O)Nc2nc(Oc3ccccc3)cc(C3CCC(F)(F)CC3)n2)cn1]

### Step 1: N-[4-(4,4-difluorocyclohexen-1-yl)-6-phenoxy-pyrimidin-2-yl]-1-methyl-pyrazole-4-sulfonamide

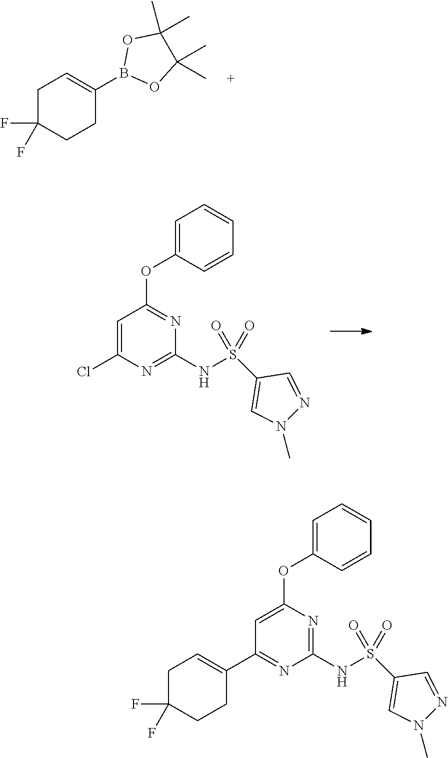 [SMILES: CC1(C)OB(C2=CCC(F)(F)CC2)OC1(C)C ; Cn1cc(S(=O)(=O)Nc2nc(Cl)cc(Oc3ccccc3)n2)cn1 ; Cn1cc(S(=O)(=O)Nc2nc(Oc3ccccc3)cc(C3=CCC(F)(F)CC3)n2)cn1]

A heterogeneous mixture of 2-(4,4-difluorocyclohexen-1-yl)-4,4,5,5-tetramethyl-1,3,2-dioxaborolane (approximately 43.94 mg, 0.1800 mmol), N-(4-chloro-6-phenoxy-pyrimidin-2-yl)-1-methyl-pyrazole-4-sulfonamide (36.58 mg, 0.10 mmol), cesium carbonate (approximately 162.9 mg, 0.5000 mmol), and bis(triphenylphosphine)palladium(II) dichloride (approximately 14.04 mg, 0.02000 mmol) in 1.00 mL dioxane/water (10:1, 0.1M) was heated to 100° C. for 12 hours. The organic layers were separated from the aqueous layer and the solvent was evaporated under a stream of air. To the crude residue was added 0.020 mL of acetic acid and 0.50 mL of dimethyl sulfoxide. The resulting mixture was filtered and separated by reverse phase chromatography (gradient: 1-99% CH₃CN in H₂Owith 0.1% hydrochloric acid) to give N-[4-(4,4-difluorocyclohexen-1-yl)-6-phenoxy-pyrimidin-2-yl]-1-methyl-pyrazole-4-sulfonamide (10 mg, 22%). ESI-MS m/z calc. 447.11768, found 448.36 (M+1)⁺; Retention time: 1.7 minutes; LC method A.

### Step 2: N-[4-(4,4-Difluorocyclohexyl)-6-phenoxy-pyrimidin-2-yl]-1-methyl-pyrazole-4-sulfonamide (Compound 264)

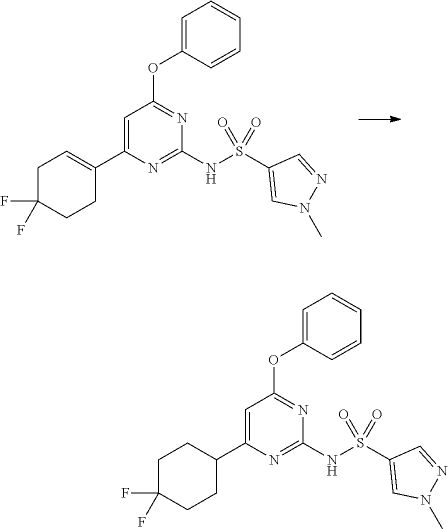 [SMILES: Cn1cc(S(=O)(=O)Nc2nc(Oc3ccccc3)cc(C3=CCC(F)(F)CC3)n2)cn1 ; Cn1cc(S(=O)(=O)Nc2nc(Oc3ccccc3)cc(C3CCC(F)(F)CC3)n2)cn1]

To a solution of N-[4-(4,4-difluorocyclohexen-1-yl)-6-phenoxy-pyrimidin-2-yl]-1 -methyl-pyrazole-4-sulfonamide (10 mg, 0.02235 mmol) in 1.0 mL of methanol and 2.0 mL of ethyl acetate was added 10% palladium on carbon (approximately 23.78 mg of 10% w/w, 0.02235 mmol). The solution was stirred under an atmosphere of hydrogen gas at 23° C. for 14 hours. The solvent was removed in vacuo and the crude residue was dissolved in 1.0 mL of dimethyl sulfoxide and filtered. The purification was carried out by reverse phase chromatography (eluent: acetonitrile/water with 0.1% hydrochloric acid). N-[4-(4,4-difluorocyclohexyl)-6-phenoxy-pyrimidin-2-yl]-1-methyl-pyrazole-4-sulfonamide (9.4 mg, 93%). ESI-MS m/z calc. 449.13333, found 449.88 (M+1)⁺; Retention time: 1.55 minutes; LC method A.

### Example 85: Preparation of Compound 265

### Step 1: N-[4-(cyclohexen-1-yl)-6-phenoxy-pyrimidin-2-yl]-1-methyl-pyrazole-4-sulfonamide (Compound 266)

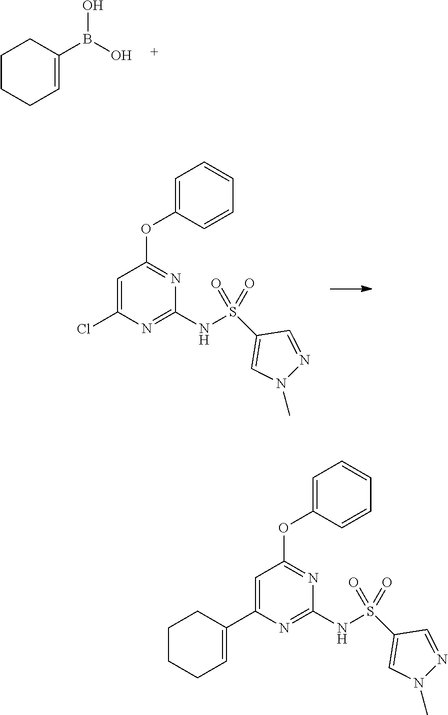 [SMILES: OB(O)C1=CCCCC1 ; Cn1cc(S(=O)(=O)Nc2nc(Cl)cc(Oc3ccccc3)n2)cn1 ; Cn1cc(S(=O)(=O)Nc2nc(Oc3ccccc3)cc(C3=CCCCC3)n2)cn1]

A heterogeneous mixture of cyclohexen-1-ylboronic acid (approximately 22.67 mg, 0.1800 mmol), N-(4-chloro-6-phenoxy-pyrimidin-2-yl)-1-methyl-pyrazole-4-sulfonamide (36.58 mg, 0.10 mmol), cesium carbonate (approximately 162.9 mg, 0.5000 mmol), and dichloroPdPPh₃ (approximately 14.04 mg, 0.02000 mmol) in 1.00 mL dioxane/water (10:1, 0.1M) was heated to 100° C. for 12 hours. The organic layers were separated from the aqueous layer and the solvent concentrated under a stream of air. To the crude residue was added 0.020 mL of acetic acid and 0.50 mL of dimethyl sulfoxide. The resulting mixture was filtered and separated by reverse phase chromatography (gradient: 1-99% CH₃CN in H₂Owith 0.1% hydrochloric acid). N-[4-(cyclohexen-1-yl)-6-phenoxy-pyrimidin-2-yl]-1-methyl-pyrazole-4-sulfonamide (5.5 mg, 13%). ESI-MS m/z calc. 411.1365, found 412.4 (M+1)⁺; Retention time: 1.85 minutes; LC method A.

### Step 2: N-(4-cyclohexyl-6-phenoxy-pyrimidin-2-yl)-1-methyl-pyrazole-4-sulfonamide (Compound 265)

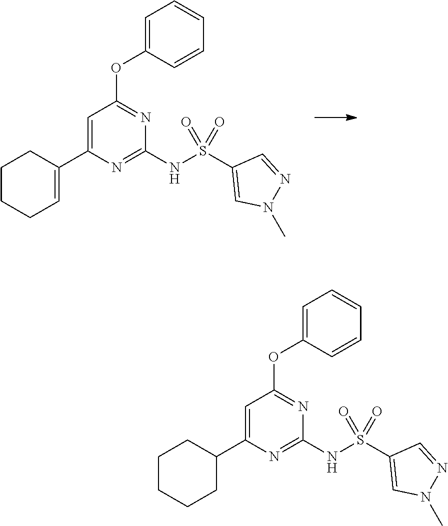 [SMILES: Cn1cc(S(=O)(=O)Nc2nc(Oc3ccccc3)cc(C3=CCCCC3)n2)cn1 ; Cn1cc(S(=O)(=O)Nc2nc(Oc3ccccc3)cc(C3CCCCC3)n2)cn1]

To a solution of N-[4-(cyclohexen-1-yl)-6-phenoxy-pyrimidin-2-yl]-1-methyl-pyrazole-4-sulfonamide (10 mg, 0.02430 mmol) in 1.0 mL of methanol and 2.0 mL of ethyl acetate was added 10% palladium on carbon (approximately 25.86 mg of 10% w/w, 0.02430 mmol). The solution was stirred under an atmosphere of hydrogen gas at 23° C. for 14 hours. The solvent was removed in vacuo and the crude residue was dissolved in 1.0 mL of dimethyl sulfoxide and filtered. The separation was carried out by reverse phase chromatography (eluent: acetonitrile/water with 0.1% hydrochloric acid). N-(4-cyclohexyl-6-phenoxy-pyrimidin-2-yl)-1 -methyl-pyrazole-4-sulfonamide (5.9 mg, 58%). ESI-MS m/z calc. 413.15216, found 414.28 (M+1)⁺; Retention time: 1.18 minutes; LC method A.

### Example 86: Preparation of Compound 267

### Step 1: N-[4-(6,6-Dimethyl-2,5-dihydropyran-4-yl)-6-phenoxy-pyrimidin-2-yl]-1 -methyl-pyrazole-4-sulfonamide

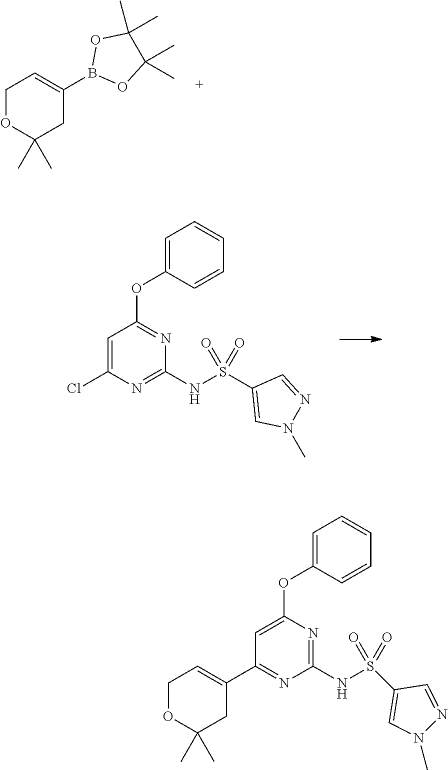 [SMILES: CC1(C)CC(B2OC(C)(C)C(C)(C)O2)=CCO1 ; Cn1cc(S(=O)(=O)Nc2nc(Cl)cc(Oc3ccccc3)n2)cn1 ; Cn1cc(S(=O)(=O)Nc2nc(Oc3ccccc3)cc(C3=CCOC(C)(C)C3)n2)cn1]

A heterogeneous mixture of 2-(2,2-dimethyl-3,6-dihydro-2H-pyran-4-yl)-4,4,5,5-tetramethyl-1,3,2-dioxaborolane (approximately 42.86 mg, 0.1800 mmol), N-(4-chloro-6-phenoxy-pyrimidin-2-yl)-1-methyl-pyrazole-4-sulfonamide (36.58 mg, 0.10 mmol), cesium carbonate (approximately 162.9 mg, 0.5000 mmol), and bis(triphenylphosphine)palladium(II) dichloride(approximately 14.04 mg, 0.02000 mmol) in 1.00 mL dioxane/water (10:1, 0.1M) was heated to 100° C. for 12 hours. The organic layer was separated from the aqueous layer and the solvent evaporated under a stream of air. To the crude residue was added 0.020 mL of acetic acid and 0.50 mL of dimethyl sulfoxide. The resulting mixture was filtered and separated by reverse phase chromatography (gradient: 1-99% CH₃CN in H₂O with 0.1% hydrochloric acid) to give N-[4-(6,6-dimethyl-2,5-dihydropyran-4-yl)-6-phenoxy-pyrimidin-2-yl]-1-methyl-pyrazole-4-sulfonamide (10 mg, 23%). ESI-MS m/z calc. 441.14706, found 442.41 (M+1)⁺; Retention time: 1.49 minutes; LC method A.

### Step 2: N-[4-(2,2-Dimethyltetrahydropyran-4-yl)-6-phenoxy-pyrimidin-2-yl]-1 -methyl-pyrazole-4-sulfonamide (Compound 267)

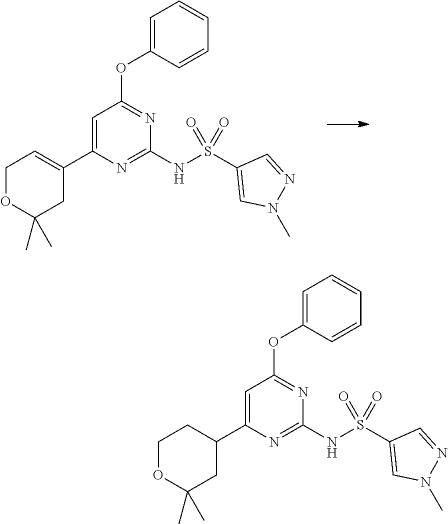 [SMILES: Cn1cc(S(=O)(=O)Nc2nc(Oc3ccccc3)cc(C3=CCOC(C)(C)C3)n2)cn1 ; Cn1cc(S(=O)(=O)Nc2nc(Oc3ccccc3)cc(C3CCOC(C)(C)C3)n2)cn1]

To a solution of N-[4-(6,6-dimethyl-2,5-dihydropyran-4-yl)-6-phenoxy-pyrimidin-2-yl]-1-methyl-pyrazole-4-sulfonamide (10 mg, 0.02265 mmol) in 1.0 mL of methanol and 2.0 mL of ethyl acetate was added 10% palladium on carbon (approximately 24.10 mg of 10% w/w, 0.02265 mmol). The solution was stirred under an atmosphere of hydrogen gas at 23° C. for 14 hours. The solvent was removed in vacuo and the crude residue was dissolved in 1.0 mL of dimethyl sulfoxide and filtered. The separation was carried out by reverse phase chromatography (eluent: acetonitrile/water with 0.1% hydrochloric acid) to give N-[4-(2,2-dimethyltetrahydropyran-4-yl)-6-phenoxy-pyrimidin-2-yl]-1-methyl-pyrazole-4-sulfonamide (4.8 mg, 48%). ESI-MS m/z calc. 443.16272, found 444.17 (M+1)⁺; Retention time: 1.35 minutes; LC method A.

### Example 87: Preparation of Compound 268

### Step 1: 1-methyl-N-(4-phenoxy-6-phenyl-pyrimidin-2-yl)pyrazole-4-sulfonamide (Compound 268)

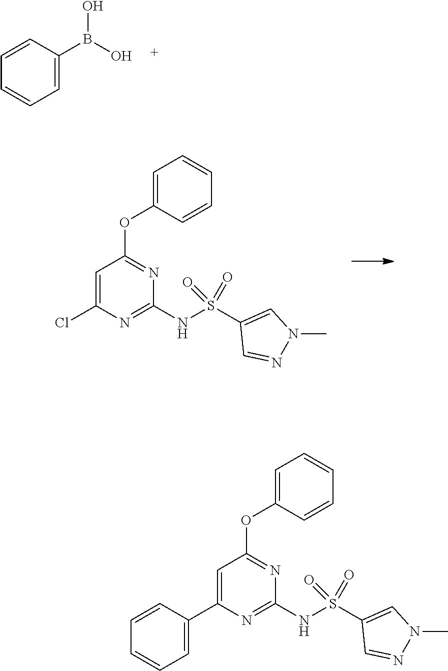 [SMILES: OB(O)c1ccccc1 ; Cn1cc(S(=O)(=O)Nc2nc(Cl)cc(Oc3ccccc3)n2)cn1 ; Cn1cc(S(=O)(=O)Nc2nc(Oc3ccccc3)cc(-c3ccccc3)n2)cn1]

To a mixture of N-(4-chloro-6-phenoxy-pyrimidin-2-yl)-1-methyl-pyrazole-4-sulfonamide (100 mg, 0.2734 mmol), phenylboronic acid (approximately 66.67 mg, 0.5468 mmol), sodium carbonate (approximately 547.0 μL of 2 M, 1.094 mmol) in DMF (2 mL) was added, [1,1′-Bis(diphenylphosphino)ferrocene]dichloropalladium(II), complex with dichloromethane. The mixture was thoroughly flushed with nitrogen and heated at 100° C. for 1 hour. The reaction mixture was filtered and purified by reverse phase HPLC using a gradient of 30-99% acetonitrile in water using HCl as a modifier. The product was repurified by reverse phase HPLC using a gradient of 25-75% acetonitrile in water using 5 mM ammonium formate as a modifier to give 1-methyl-N-(4-phenoxy-6-phenyl-pyrimidin-2-yl)pyrazole-4-sulfonamide (42 mg, 38%) as a white solid. ¹H NMR (400 MHz, DMSO) δ 11.60 (s, 1H), 8.10 (dd, J=6.5, 3.0 Hz, 2H), 7.79 (s, 1H), 7.59-7.49 (m, 5H), 7.42 (s, 1H), 7.35 (t, J=7.4 Hz, 1H), 7.32-7.23 (m, 3H), 3.76 (s, 3H). ESI-MS m/z calc. 407.10522, found 408.0 (M+1)⁺; Retention time: 1.65 minutes; LC method A.

### Example 88: Preparation of Compound 269

### Step 1: N-[4-(2-isopropylphenyl)-6-methylsulfanyl-pyrimidin-2-yl]-1-methyl-pyrazole-4-sulfonamide

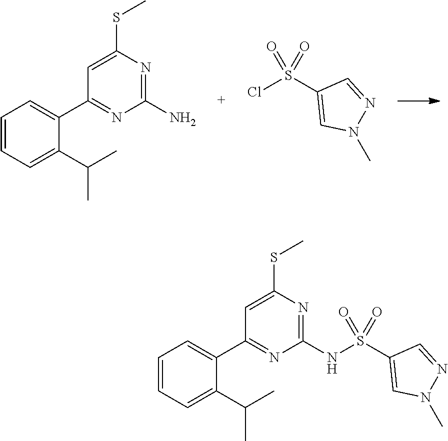

To a heat-gun-dried 20 mL microwave vial equipped with a magnetic stir bar were added 4-(2-isopropylphenyl)-6-methylsulfanyl-pyrimidin-2-amine (654.2 mg, 2.522 mmol) and dimethylformamide (8 mL), and this mixture was cooled to 0° C. 60% NaH (400.0 mg, 10.00 mmol) was added in one portion, and the reaction mixture was warmed to room temperature over 15 minutes. The mixture was cooled to 0° C., upon which 1-methylpyrazole-4-sulfonyl chloride (700.0 mg, 3.876 mmol) was added in one portion. This solution was stirred at room temperature for 4 h, then quenched by a slow transfer onto ice-cold 1 N HCl (30 mL). The mixture was extracted with ethyl acetate (3×40 mL). The combined organic extracts were washed with water (50 mL) and saturated aqueous sodium chloride solution (40 mL), then dried over sodium sulfate, filtered, and evaporated in vacuo. This crude product was purified by silica gel chromatography (24 g of silica, 0 to 50% gradient of ethyl acetate/hexanes) to give five batches of product, which contained recovered starting material (24.5%), 5-chloro-N-[4-(2-isopropylphenyl)-6-methylsulfanyl-pyrimidin-2-yl]-1-methyl-pyrazole-4-sulfonamide (139.8 mg, 13%), and N-[4-(2-isopropylphenyl)-6-methylsulfanyl-pyrimidin-2-yl]-1-methyl-pyrazole-4-sulfonamide (309.6 mg, 30%)¹H NMR (400 MHz, Chloroform-d) δ 7.91 (s, 1H), 7.90 (s, 1H), 7.45-7.41 (m, 2H), 7.26-7.22 (m, 1H), 7.22-7.18 (m, 1H), 6.84 (s, 1H), 3.85 (s, 3H), 3.21 (heptet, J=6.9 Hz, 1H), 2.55 (s, 3H), 1.20 (d, J=6.9 Hz, 6H). ESI-MS m/z calc. 403.11368, found 404.2 (M+1)⁺; Retention time: 0.62 minutes; LC method D.

### Step 2: N-[4-(2-isopropylphenyl)-6-methylsulfonyl-pyrimidin-2-yl]-1-methyl-pyrazole-4-sulfonamide

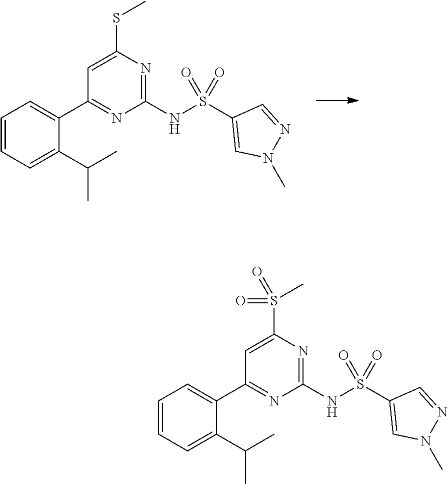 [SMILES: CSc1cc(-c2ccccc2C(C)C)nc(NS(=O)(=O)c2cnn(C)c2)n1 ; CC(C)c1ccccc1-c1cc(S(C)(=O)=O)nc(NS(=O)(=O)c2cnn(C)c2)n1]

To a 20 mL vial equipped with a magnetic stir bar, N-[4-(2-isopropylphenyl)-6-methylsulfanyl-pyrimidin-2-yl]-1-methyl-pyrazole-4-sulfonamide (309.6 mg, 0.7672 mmol) and dichloromethane (7.0 mL) were added, followed by m-CPBA (400.0 mg, 1.785 mmol). This solution was stirred at room temperature for 30 minutes. The reaction mixture was quenched with solid sodium thiosulfate (900.0 mg, 5.692 mmol). This mixture was stirred for another 30 min at room temperature. The reaction mixture was diluted with dichloromethane (7 mL), then washed with water (2×4 mL) and saturated aqueous sodium chloride solution (4 mL). The organic layer was then dried over sodium sulfate, filtered, and evaporated in vacuo. This crude product was purified by silica gel chromatography (12 g of silica, 0 to 70% gradient of ethyl acetate/hexanes) to give N-[4-(2-isopropylphenyl)-6-methylsulfonyl-pyrimidin-2-yl]-1-methyl-pyrazole-4-sulfonamide (309.0 mg, 92%). ESI-MS m/z calc. 435.1035, found 436.2 (M+1)⁺; Retention time: 0.54 minutes; LC method D.

### Step 3: N-[4-(2-isopropylphenyl)-6-phenoxy-pyrimidin-2-yl]-1-methyl-pyrazole-4-sulfonamide (Compound 269)

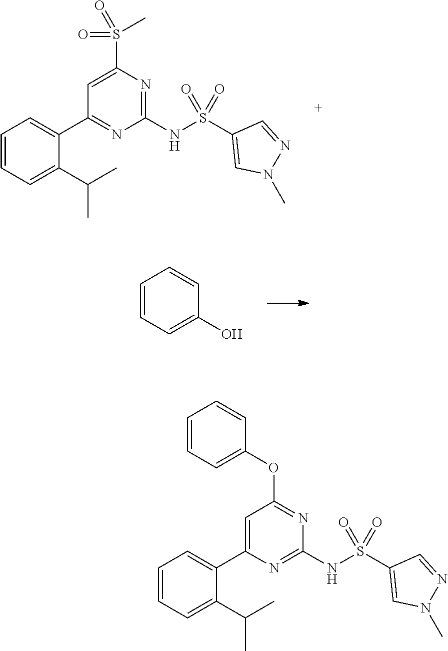 [SMILES: CC(C)c1ccccc1-c1cc(S(C)(=O)=O)nc(NS(=O)(=O)c2cnn(C)c2)n1 ; Oc1ccccc1 ; CC(C)c1ccccc1-c1cc(Oc2ccccc2)nc(NS(=O)(=O)c2cnn(C)c2)n1]

To a 10 mL vial equipped with a magnetic stir bar, N-[4-(2-isopropylphenyl)-6-methylsulfonyl-pyrimidin-2-yl]-1-methyl-pyrazole-4-sulfonamide (15.61 mg, 0.03585 mmol) (15.6 mg, 0.03585 mmol), N-methylpyrrolidinone (500 μL) and phenol (13.50 mg, 12.74 μL, 0.1434 mmol) were added, followed by potassium carbonate (25.00 mg, 0.1809 mmol) (25.0 mg, 0.1809 mmol). This mixture was stirred at 120° C. for 7 hours. The reaction mixture was then cooled to room temperature, quenched with 1 N HCl (1 mL), and extracted with ethyl acetate (3×1 mL). The combined organic extracts were washed with water (2×2 mL) and saturated aqueous sodium chloride solution (2 mL), then dried over sodium sulfate, filtered, and evaporated in vacuo. Purification by reverse phase HPLC (1-99% acetonitrile in water using HCl as a modifier) gave the desired product N-[4-(2-isopropylphenyl)-6-phenoxy-pyrimidin-2-yl]-1-methyl-pyrazole-4-sulfonamide (4.2 mg, 26%). ESI-MS m/z calc. 449.15216, found 450.2 (M+1)⁺; Retention time: 1.8 minutes; LC method A.

### Example 89: Preparation of Compound 270

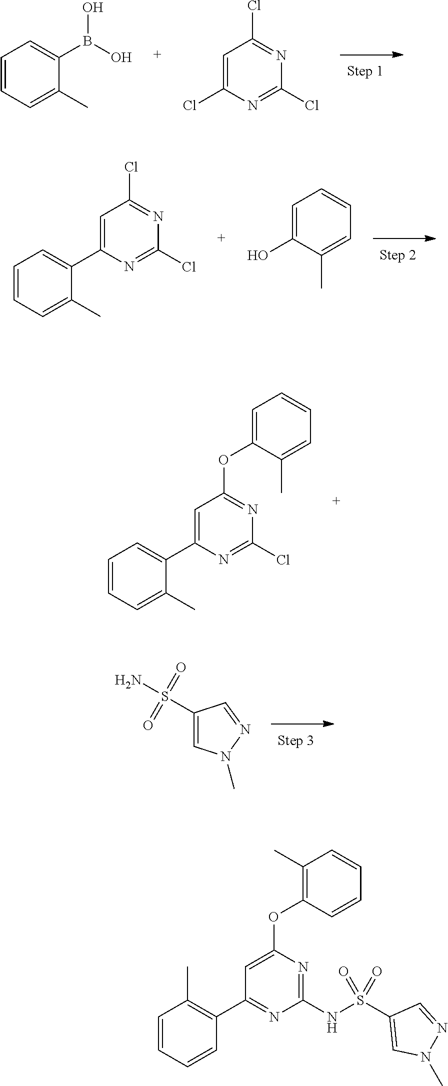

### Step 1: 2,4-Dichloro-6-(o-tolyl)pyrimidine

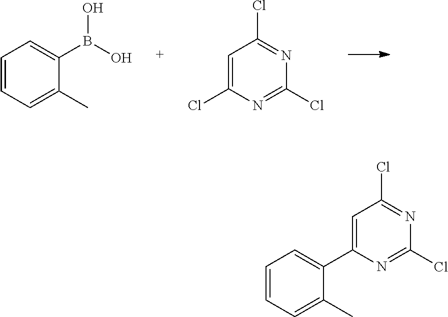

A mixture of 2,4,6-trichloropyrimidine (5.60 g, 30.5 mmol) in DME (280 mL) and 2 N aqueous sodium carbonate (46 mL, 92 mmol) was degassed by bubbling with nitrogen gas for 10 minutes. Added o-tolylboronic acid (4.15 g, 30.5 mmol), triphenylphosphine (801 mg, 3.05 mmol) and palladium acetate (343 mg, 1.53 mmol) and heated in an oil bath set at 85° C. for 17.5 hours. This was cooled to room temperature and concentrated under reduced pressure to remove most of the DME. The residue was transferred to a 1.0-L separatory funnel with water (about 250 mL) and extracted with EtOAc (3×200 mL). The combined organic layers were washed with brine, dried over Na₂SO₄, filtered and concentrated under reduced pressure. The crude material was combined with another reaction run on 1 g scale and purified by silica-gel column chromatography, eluting with 50% heptanes in toluene. The oil solidified to a white solid. A total of (0.24 g+4.42 g) 4.66 g (54% yield) of 2,4-dichloro-6-(o-tolyl)pyrimidine product was obtained.

### Step 2: 2-Chloro-4-(2-methylphenoxy)-6-(o-tolyl)pyrimidine

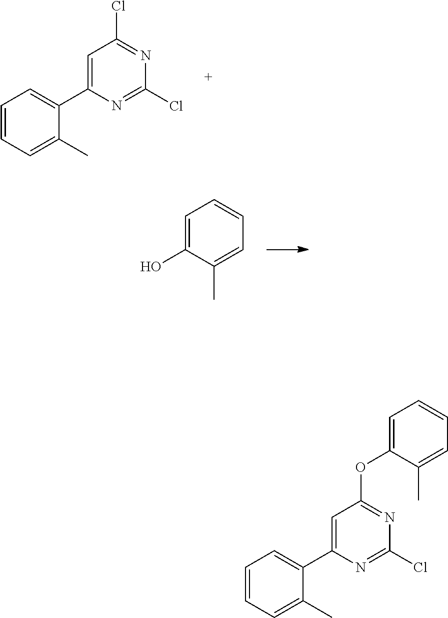

A mixture of 2,4-dichloro-6-(o-tolyl)pyrimidine (4.14 g, 17.3 mmol), o-cresol (1.87 g, 17.3 mmol) and cesium carbonate (8.46 g, 26.0 mmol) in acetonitrile (90 mL) was heated in an oil bath set at 30° C. overnight. The mixture was cooled to room temperature, filtered, washed with EtOAc and then concentrated under reduced pressure. The material was combined with another reaction run on 1 g scale. The desired material was recrystallized from a mixture of heptanes (50 mL) and EtOAc (15 mL). The solids were washed with heptanes and dried under high vacuum to give 3.99 g of the desired product 2-chloro-4-(2-methylphenoxy)-6-(o-tolyl)pyrimidine as a white solid (70% combined yield). ESI-MS m/z calc. 310.08728, found 311.1 (M+1)⁺; Retention time: 5.75 minutes (LC method G). ¹H NMR (300 MHz, CDCl₃) ppm 2.22 (s, 3H), 2.41 (s, 3H), 6.74 (s, 1H), 7.07-7.15 (m, 1H), 7.17-7.39 (m, 6H), 7.40-7.46 (m, 1H).

### Step 3: 1-methyl-N-[4-(2-methylphenoxy)-6-(o-tolyl)pyrimidin-2-yl]pyrazole-4-sulfonamide (Compound 270)

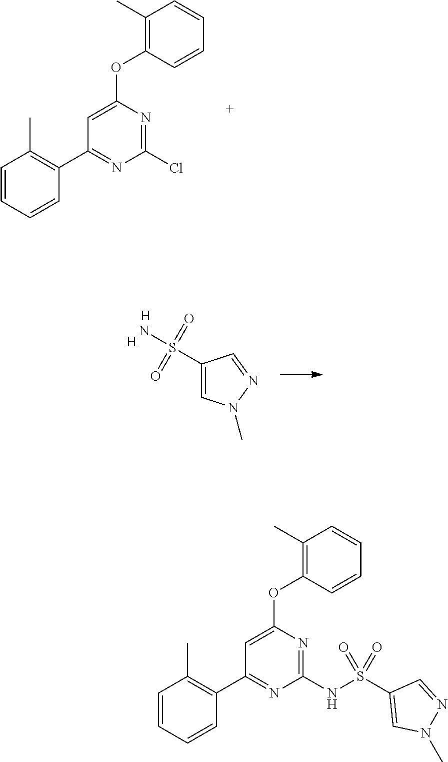

Nitrogen was bubbled through a mixture of 1-methylpyrazole-4-sulfonamide (78.8 mg, 0.4889 mmol), 2-chloro-4-(2-methylphenoxy)-6-(o-tolyl)pyrimidine (150 mg, 0.4827 mmol), Xantphos (51 mg, 0.08814 mmol), Pd (OAc)₂ (35 mg, 0.1559 mmol) and cesium carbonate (approximately 318.0 mg, 0.9760 mmol) in 1,4-dioxane (4.200 mL) for 5 minutes at room temperature. The reaction was then heated at 120° C. for 4 hours. The crude product was filtered through a Celite pad, concentrated, and purified on reverse phase HPLC (HCl modifier, 25-75% ACN-H₂O) to give 1-methyl-N-[4-(2-methylphenoxy)-6-(o-tolyl)pyrimidin-2-yl]pyrazole-4-sulfonamide (127.4 mg, 61%). ¹H NMR (400 MHz, DMSO) δ 11.62 (s, 1H), 7.56 (s, 1H), 7.42 (d, J=7.5 Hz, 2H), 7.40-7.34 (m, 2H), 7.34-7.27 (m, 3H), 7.22 (d, J=7.8 Hz, 2H), 6.77 (s, 1H), 3.75 (s, 3H), 2.36 (s, 3H), 2.15 (s, 3H). ESI-MS m/z calc. 435.1365, found 436.0 (M+1)⁺; Retention time: 1.69 minutes; LC method A.

### Example 90: Preparation of Compound 271

### Step 1: N-[4-(2,6-Dimethylphenyl)-6-phenoxy-pyrimidin-2-yl]-1-methyl-pyrazole-4-sulfonamide (Compound 271)

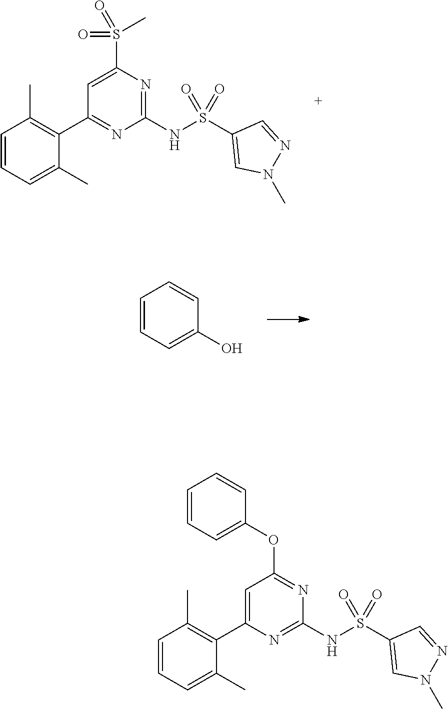 [SMILES: Cc1cccc(C)c1-c1cc(S(C)(=O)=O)nc(NS(=O)(=O)c2cnn(C)c2)n1 ; Oc1ccccc1 ; Cc1cccc(C)c1-c1cc(Oc2ccccc2)nc(NS(=O)(=O)c2cnn(C)c2)n1]

To a 10 mL vial equipped with a magnetic stir bar, N-[4-(2,6-dimethylphenyl)-6-methylsulfonyl-pyrimidin-2-yl]-1-methyl-pyrazole-4-sulfonamide (25.00 mg, 0.05931 mmol) (25.0 mg, 0.0593 mmol), N-methylpyrrolidinone (800 μL) and phenol (21.65 mg, 20.42 μL, 0.23 mmol) were added, followed by potassium carbonate (31.79 mg, 0.23 mmol) (31.8 mg, 0.23 mmol). This mixture was stirred at 110° C. for 15 hours. The reaction mixture was then cooled to room temperature, quenched with 1 N HCl (1 mL), and extracted with ethyl acetate (3×1 mL). The combined organic extracts were washed with water (2×2 mL) and saturated aqueous sodium chloride solution (2 mL), then dried over sodium sulfate, filtered, and evaporated in vacuo. This was then purified by reverse phase HPLC (1-99% acetonitrile in water using HCl as a modifier) to give the desired product N-[4-(2,6-dimethylphenyl)-6-phenoxy-pyrimidin-2-yl]-1-methyl-pyrazole-4-sulfonamide (13.4 mg, 52%). ESI-MS m/z calc. 435.1365, found 436.2 (M+1)⁺; Retention time: 1.61 minutes; LC method A.

### Example 91: Preparation of Compound 272, Compound 273, and Compound 274

### Step 1: N-[4-[2-chloro-3-(4-methylpiperazin-1-yl)phenoxy]-6-(2-cyclobutylphenyl)pyrimidin-2-yl]-1-methyl-pyrazole-4-sulfonamide (Compound 272), N-[4-[2-chloro-3-(4-methylpiperazin-1-yl)phenoxy]-6-(2-isobutylphenyl)pyrimidin-2-yl]-1-methyl-pyrazole-4-sulfonamide (Compound 273) and N-[4-[2-chloro-3-(4-methylpiperazin-1-yl)phenoxy]-6-(2-isopropylphenyl)pyrimidin-2-yl]-1-methyl-pyrazole-4-sulfonamide (Compound 274)

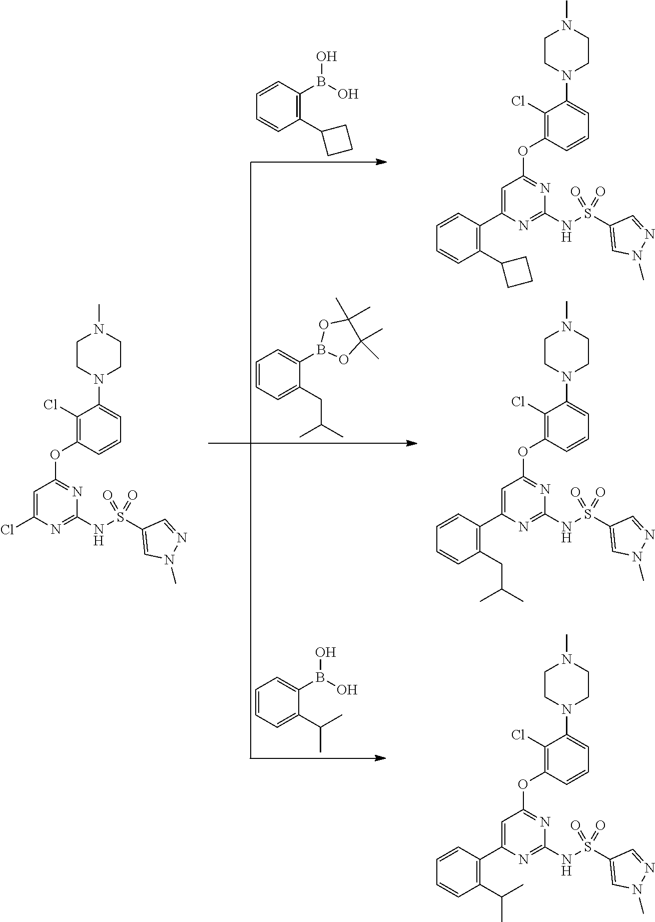

A microwave vial was charged with N-[4-chloro-6-[2-chloro-3-(4-methylpiperazin-1-yl)phenoxy]pyrimidin-2-yl]-1-methyl-pyrazole-4-sulfonamide (prepared in a manner analogous to that described above, 17.1 mg, 0.03431 mmol), Pd(dppf)Cl₂(4.2 mg, 0.005143 mmol), (2-cyclobutylphenyl)boronic acid (7.7 mg, 0.04375 mmol), dioxane (0.7 mL), and K₂CO₃ (70 μL of 2 M, 0.1400 mmol) and the reaction mixture was sparged with nitrogen for 1 minute and then microwaved at 120° C. for 30 minutes. The solutions was filtered and the filtrate dissolved in 0.8 mL MeOH, and purified by reverse phase chromatography using a 15 min gradient of 200% MeCN in water to 80% MeCN (HCl modifier) to give N-[4-[2-chloro-3-(4-methylpiperazin-1-yl)phenoxy]-6-(2-cyclobutylphenyl)pyrimidin-2-yl]-1-methyl-pyrazole-4-sulfonamide (hydrochloride salt) (7.6 mg, 350%)¹H NMR (400 MHz, DMSO-d₆) δ 10.86 (s, 1H), 7.72 (s, 1H), 7.55-7.41 (m, 3H), 7.35-7.28 (m, 2H), 7.24 (td, J=8.0, 1.4 Hz, 2H), 7.16 (s, 1H), 6.74 (s, 1H), 3.92-3.82 (m, 1H), 3.79 (s, 3H), 3.56-3.47 (m, 4H), 3.29-3.08 (m, 4H), 2.83 (d, J=4.7 Hz, 3H), 2.15-1.59 (in, 6H). ESI-MS m/z calc. 593.1976, found 594.2 (M+1)⁺; Retention time: 1.43 minutes; LC method A.

A microwave vial was charged with N-[4-chloro-6-[2-chloro-3-(4-methylpiperazin-1-yl)phenoxy]pyrimidin-2-yl]-1-methyl-pyrazole-4-sulfonamide (17.1 mg, 0.03431 mmol), 2-(2-isobutylphenyl)-4,4,5,5-tetramethyl-1,3,2-dioxaborolane (9.8 mg, 0.03767 mmol), Pd(dppf)Cl₂(4.2 mg, 0.005143 mmol), dioxane (0.7 mL), and K₂CO₃ (70 μL of 2 M, 0.1400 mmol) and the reaction mixture was sparged with nitrogen for 1 minute and then microwaved at 120° C. for 30 minutes. The solution was filtered and the filtrate dissolved in 0.8 mL MeOH, and purified by reverse phase chromatography using a 15 minutes gradient of 20% MeCN in water to 80% MeCN (HCl modifier) to give N-[4-[2-chloro-3-(4-methylpiperazin-1-yl)phenoxy]-6-(2-isobutylphenyl)pyrimidin-2-yl]-1-methyl-pyrazole-4-sulfonamide (hydrochloride salt) (2.7 mg, 12%)¹H NMR (400 MHz, DMSO-d₆) δ 10.46 (s, 1H), 7.72 (s, 1H), 7.51 (t, J=8.1 Hz, 1H), 7.46-7.38 (m, 1H), 7.37-7.28 (m, 3H), 7.28-7.19 (m, 2H), 7.13 (s, 1H), 6.83 (s, 1H), 3.78 (s, 3H), 3.55-3.49 (m, 4H), 3.32-3.04 (m, 4H), 2.85 (d, J=4.6 Hz, 3H), 2.70-2.64 (m, 2H), 1.60 -1.49 (m, 1H), 0.70 (d, J=6.6 Hz, 6H). ESI-MS m/z calc. 595.21326, found 596.51 (M+1)⁺; Retention time: 1.46 minutes; LC method A.

A microwave vial was charged with N-[4-chloro-6-[2-chloro-3-(4-methylpiperazin-1-yl)phenoxy]pyrimidin-2-yl]-1-methyl-pyrazole-4-sulfonamide (17.1 mg, 0.03431 mmol), (2-isopropylphenyl)boronic acid (6.7 mg, 0.04085 mmol), Pd(dppf)C₁₂(4.2 mg, 0.005143 mmol), K₂CO₃ (70 μL of 2 M, 0.1400 mmol), and dioxane (0.7 mL) and the reaction mixture was sparged with nitrogen for 1 minute and then microwaved at 120° C. for 30 minutes. The solution was filtered and the filtrate dissolved in 0.8 mL MeOH, and purified by reverse phase chromatography using a 15 minute gradient of 20% MeCN in water to 80% MeCN (HCl modifier) to give N-[4-[2-chloro-3-(4-methylpiperazin-1-yl)phenoxy]-6-(2-isopropylphenyl)pyrimidin-2-yl]-1-methyl-pyrazole-4-sulfonamide (hydrochloride salt) (6.6 mg, 31%). ¹H NMR (400 MHz, DMSO-d₆) δ 10.79 (s, 1H), 7.65 (s, 1H), 7.52 (t, J=8.1 Hz, 1H), 7.48-7.45 (m, 1H), 7.35-7.20 (m, 5H), 7.09 (s, 1H), 6.78 (s, 1H), 3.79 (s, 3H), 3.62-3.46 (m, 4H), 3.30-3.05 (m, 6H), 2.84 (d, J=4.6 Hz, 3H), 1.14 (d, J=6.8 Hz, 6H). ESI-MS m/z calc. 581.1976, found 582.5 (M+1)⁺; Retention time: 1.38 minutes; LC method A.

### Example 92: Preparation of Compound 275

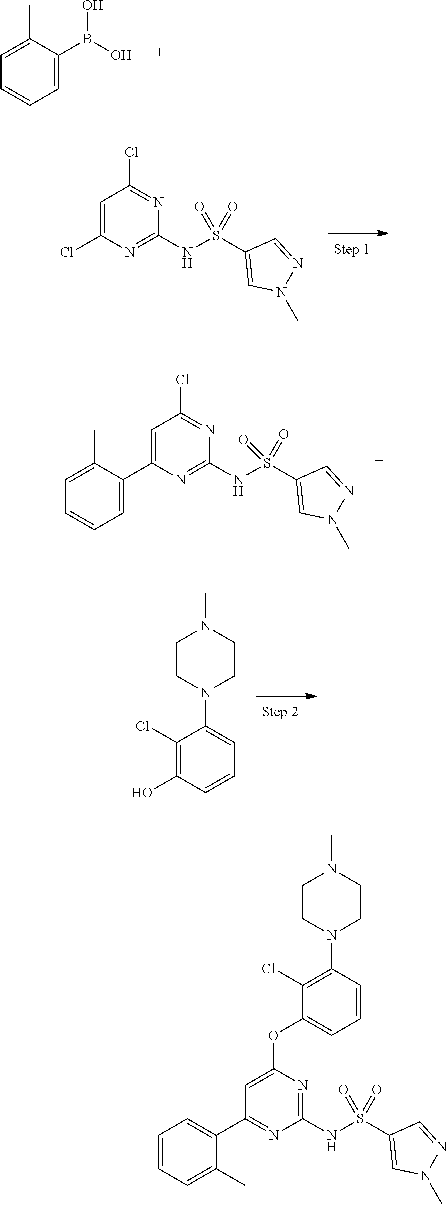

### Step 1: N-(4-chloro-6-(o-tolyl)pyrimidin-2-yl)-1-methyl-pyrazole-4-sulfonamide

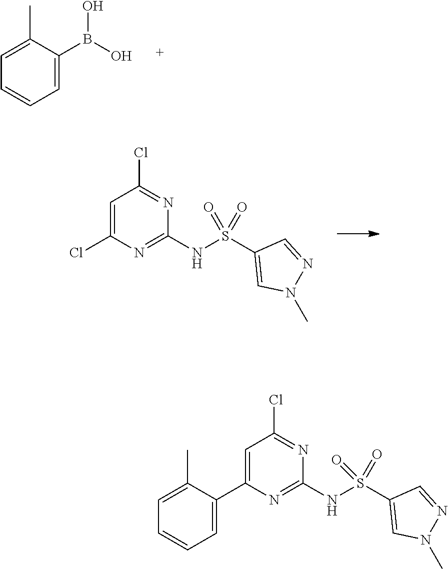

A heterogeneous solution of N-(4,6-dichloropyrimidin-2-yl)-1-methyl-pyrazole-4-sulfonamide (200 mg, 0.5614 mmol), o-tolylboronic acid (approximately 76.33 mg, 0.5614 mmol), potassium carbonate (approximately 232.7 mg, 1.684 mmol), and bis(triphenylphosphine)palladium(II) dichloride(approximately 11.82 mg, 0.01684 mmol) in dioxane (1.871 mL) and water (374.4 μL) was heated in a sealed vial at 90° C. for 16 hours. The solution was acidified with acetic acid (approximately 674.4 mg, 638.6 μL, 11.23 mmol), diluted with DMSO (1.0 mL), and filtered through a 0.45 m PTFE syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-chloro-6-(o-tolyl)pyrimidin-2-yl]-1-methyl-pyrazole-4-sulfonamide (30 mg, 14%) as a white solid. ESI-MS m/z calc. 363.05566, found 364.14 (M+1)⁺; Retention time: 0.59 minutes; LC method D.

### Step 2: N-[4-[2-chloro-3-(4-methylpiperazin-1-yl)phenoxy]-6-(o-tolyl)pyrimidin-2-yl]-1-methyl-pyrazole-4-sulfonamide (Compound 275)

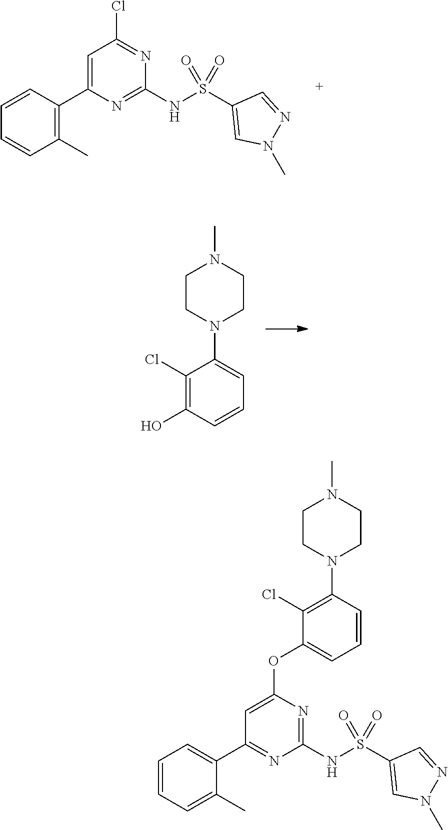 [SMILES: Cc1ccccc1-c1cc(Cl)nc(NS(=O)(=O)c2cnn(C)c2)n1 ; CN1CCN(c2cccc(O)c2Cl)CC1 ; Cc1ccccc1-c1cc(Oc2cccc(N3CCN(C)CC3)c2Cl)nc(NS(=O)(=O)c2cnn(C)c2)n1]

A heterogeneous solution of N-[4-chloro-6-(o-tolyl)pyrimidin-2-yl]-1-methyl-pyrazole-4-sulfonamide (9.096 mg, 0.0250 mmol), 2-chloro-3-(4-methylpiperazin-1-yl)phenol, and potassium carbonate (0.0750 mmol) in NMP (50.00 μL) was heated in a sealed vial to 115° C. for 16 hours. The solution was acidified with acetic acid. The sample was purified by reverse phase HPLC (Waters Sunfire C₁₈ column (100×50 mm, 10 m particle size), gradient: 1-99% acetonitrile in water (5 mM HCl) over 15.0 minutes) to afford N-[4-[2-chloro-3-(4-methylpiperazin-1-yl)phenoxy]-6-(o-tolyl)pyrimidin-2-yl]-1-methyl-pyrazole-4-sulfonamide (hydrochloride salt) as a white solid (8.3 mg, 56%). ESI-MS m/z calc. 553.16626, found 554.25 (M+1)⁺; Retention time: 1.32 minutes; LC method A.

### Example 93: Preparation of Compound 276

### Step 1: 3-(4-methylpiperazin-1-yl)phenol

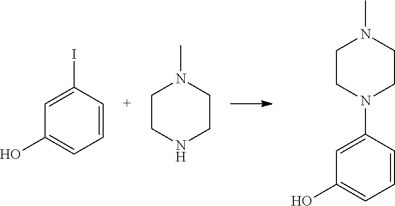

A heterogeneous solution of 3-iodophenol (2.4 g, 10.91 mmol), 1-methylpiperazine (approximately 10.93 g, 109.1 mmol), potassium tert-butoxide (approximately 2.571 g, 22.91 mmol), and Chloro(2-di-tert-butylphosphino-2′, 4′, 6′-tri-i-propyl-1,1′-biphenyl)[2-(2-aminoethyl)phenyl] palladium(II) [t-BuXPhos Palladacycle Gen. 1] (approximately 374.6 mg, 0.5455 mmol) in dioxane (64.18 mL) was heated to 60° C. for 16 hours. The crude solution was concentrated in vacuo. The crude residue was dissolved in acetic acid (approximately 39.31 g, 37.23 mL, 654.6 mmol) and concentrated in vacuo onto silica gel. The silica gel was subjected to flash column chromatography (gradient: 1 to 10% MeOH in DCM) to afford 3-(4-methylpiperazin-1-yl)phenol (1.27 g, 58%) as a dark red solid. ESI-MS m/z calc. 192.12627, found 193.08 (M+1)⁺; Retention time: 0.17 minutes; LC method D.

### Step 2: 1-Methyl-N-[4-[3-(4-methylpiperazin-1-yl)phenoxy]-6-(o-tolyl)pyrimidin-2-yl]pyrazole-4-sulfonamide (Compound 276)

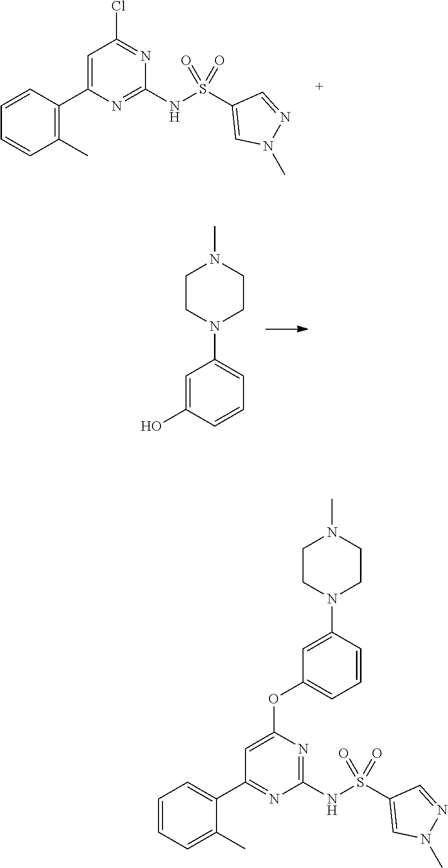 [SMILES: Cc1ccccc1-c1cc(Cl)nc(NS(=O)(=O)c2cnn(C)c2)n1 ; CN1CCN(c2cccc(O)c2)CC1 ; Cc1ccccc1-c1cc(Oc2cccc(N3CCN(C)CC3)c2)nc(NS(=O)(=O)c2cnn(C)c2)n1]

A heterogeneous solution of N-[4-chloro-6-(o-tolyl)pyrimidin-2-yl]-1-methyl-pyrazole-4-sulfonamide (9.096 mg, 0.0250 mmol), 3-(4-methylpiperazin-1-yl)phenol, and potassium carbonate (0.0750 mmol) in NMP (50.00 μL) was heated in a sealed vial to 115° C. for 16 hours. The solution was acidified with acetic acid. The sample was purified by reverse phase HPLC (Waters Sunfire C₁₈ column (100×50 mm, 10 m particle size) using a gradient: 1-99% acetonitrile in water (5 mM HCl) over 15.0 minutes) to afford 1-methyl-N-[4-[3-(4-methylpiperazin-1-yl)phenoxy]-6-(o-tolyl)pyrimidin-2-yl]pyrazole-4-sulfonamide (hydrochloride salt) as a white solid (8.3 mg, 60%). ESI-MS m/z calc. 519.20526, found 520.29 (M+1)⁺; Retention time: 1.26 minutes; LC method A.

### Example 94: Characterization of Compounds 266 and 277-311

The compounds in the following tables were prepared in a manner analogous to that described above using commercially available reagents and intermediates described herein.

| | | LCMS | | | |
|---|---|---|---|---|---|
| | | Room | | | |
| Compound | | Temperature | Calc. | | LCMS |
| number | Structure | (min) | mass | M + 1 | Method |
| 277 | 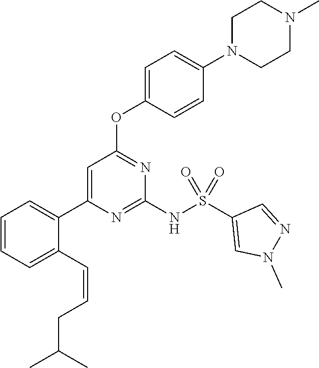 [SMILES: CC(C)C/C=C\c1ccccc1-c1cc(Oc2ccc(N3CCN(C)CC3)cc2)nc(NS(=O)(=O)c2cnn(C)c2)n1] | 1.51 | 587.268 | 588.4 | A |
| 278 | 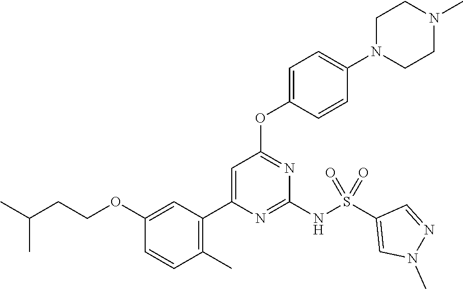 [SMILES: Cc1ccc(OCCC(C)C)cc1-c1cc(Oc2ccc(N3CCN(C)CC3)cc2)nc(NS(=O)(=O)c2cnn(C)c2)n1] | 1.57 5 | 605.278 | 606.47 | A |
| 279 | 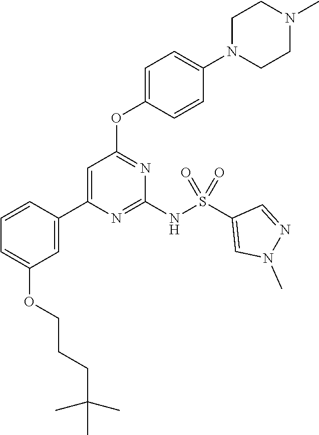 [SMILES: CN1CCN(c2ccc(Oc3cc(-c4cccc(OCCCC(C)(C)C)c4)nc(NS(=O)(=O)c4cnn(C)c4)n3)cc2)CC1] | 1.74 | 619.294 | 620.51 | A |
| 280 | 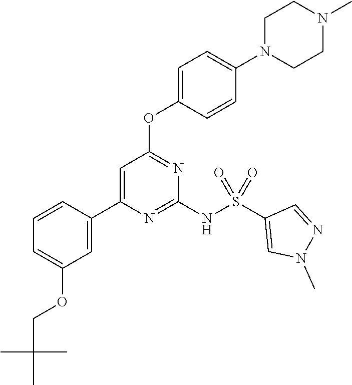 [SMILES: CN1CCN(c2ccc(Oc3cc(-c4cccc(OCC(C)(C)C)c4)nc(NS(=O)(=O)c4cnn(C)c4)n3)cc2)CC1] | 1.57 | 591.263 | 592.1 | A |
| 281 | 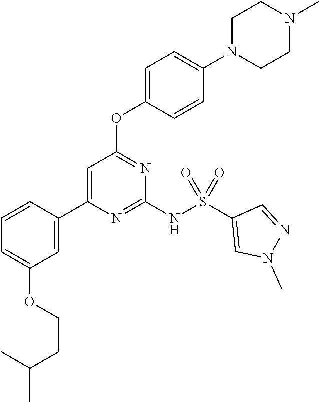 [SMILES: CC(C)CCOc1cccc(-c2cc(Oc3ccc(N4CCN(C)CC4)cc3)nc(NS(=O)(=O)c3cnn(C)c3)n2)c1] | 1.56 | 591.263 | 592.1 | A |
| 282 | 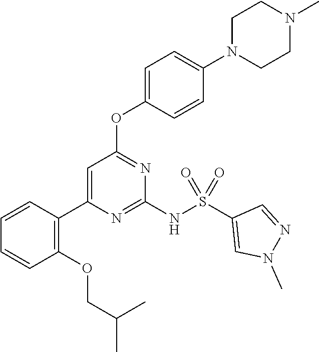 [SMILES: CC(C)COc1ccccc1-c1cc(Oc2ccc(N3CCN(C)CC3)cc2)nc(NS(=O)(=O)c2cnn(C)c2)n1] | 1.31 | 577.247 | 578.1 | A |
| 283 | 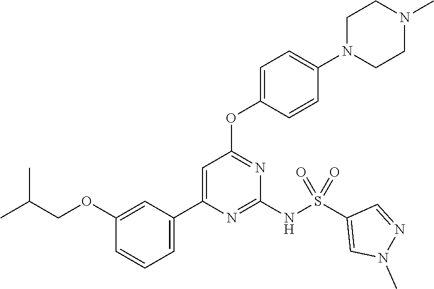 [SMILES: CC(C)COc1cccc(-c2cc(Oc3ccc(N4CCN(C)CC4)cc3)nc(NS(=O)(=O)c3cnn(C)c3)n2)c1] | 1.31 | 577.247 | 578.1 | A |
| 284 | 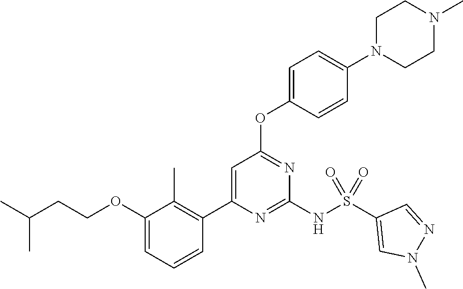 [SMILES: Cc1c(OCCC(C)C)cccc1-c1cc(Oc2ccc(N3CCN(C)CC3)cc2)nc(NS(=O)(=O)c2cnn(C)c2)n1] | 1.57 | 605.278 | 606.1 | A |
| 285 | 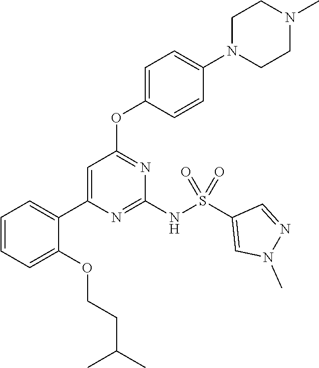 [SMILES: CC(C)CCOc1ccccc1-c1cc(Oc2ccc(N3CCN(C)CC3)cc2)nc(NS(=O)(=O)c2cnn(C)c2)n1] | 1.4 | 591.263 | 592.46 | A |
| 286 | 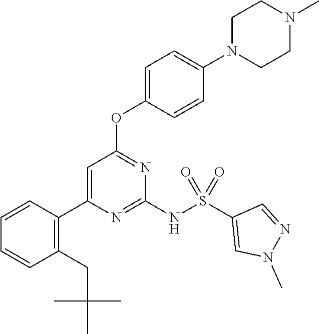 [SMILES: CN1CCN(c2ccc(Oc3cc(-c4ccccc4CC(C)(C)C)nc(NS(=O)(=O)c4cnn(C)c4)n3)cc2)CC1] | 1.37 | 575.268 | 576.44 | A |
| 287 | 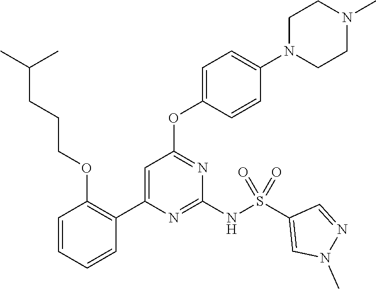 [SMILES: CC(C)CCCOc1ccccc1-c1cc(Oc2ccc(N3CCN(C)CC3)cc2)nc(NS(=O)(=O)c2cnn(C)c2)n1] | 1.5 | 605.278 | 606.43 | A |
| 288 | 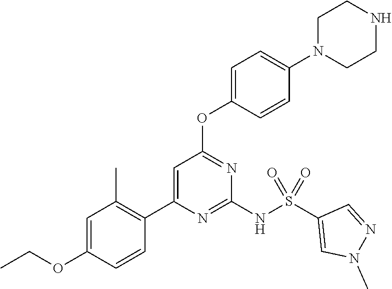 [SMILES: CCOc1ccc(-c2cc(Oc3ccc(N4CCNCC4)cc3)nc(NS(=O)(=O)c3cnn(C)c3)n2)c(C)c1] | 1.28 | 549.216 | 550.5 | A |
| 289 | 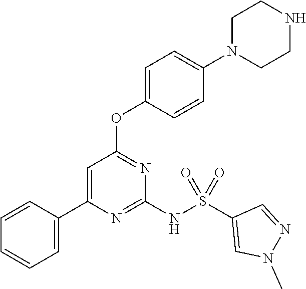 [SMILES: Cn1cc(S(=O)(=O)Nc2nc(Oc3ccc(N4CCNCC4)cc3)cc(-c3ccccc3)n2)cn1] | 1.16 | 491.174 | 492.45 | A |
| 290 | 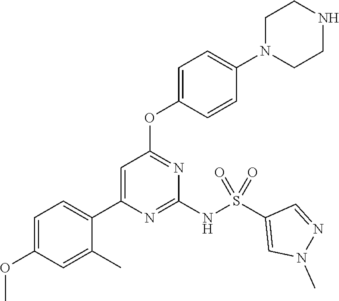 [SMILES: COc1ccc(-c2cc(Oc3ccc(N4CCNCC4)cc3)nc(NS(=O)(=O)c3cnn(C)c3)n2)c(C)c1] | 0.83 | 535.2 | 536.3 | R |
| 291 | 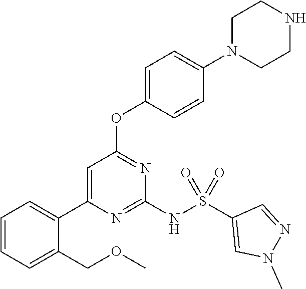 [SMILES: COCc1ccccc1-c1cc(Oc2ccc(N3CCNCC3)cc2)nc(NS(=O)(=O)c2cnn(C)c2)n1] | 1.1 | 535.2 | 536.46 | A |
| 292 | 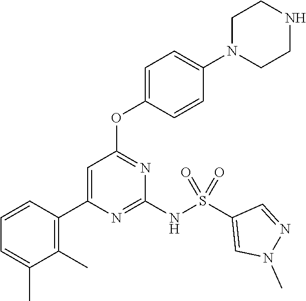 [SMILES: Cc1cccc(-c2cc(Oc3ccc(N4CCNCC4)cc3)nc(NS(=O)(=O)c3cnn(C)c3)n2)c1C] | 1.25 | 519.205 | 520.49 | A |
| 293 | 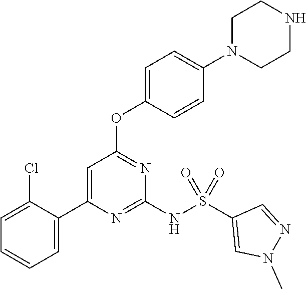 [SMILES: Cn1cc(S(=O)(=O)Nc2nc(Oc3ccc(N4CCNCC4)cc3)cc(-c3ccccc3Cl)n2)cn1] | 1.21 | 525.135 | 526.45 | A |
| 294 | 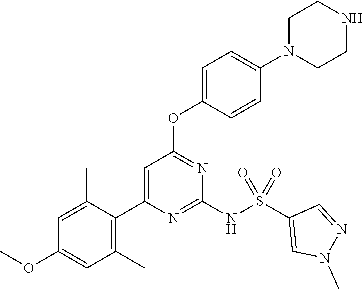 [SMILES: COc1cc(C)c(-c2cc(Oc3ccc(N4CCNCC4)cc3)nc(NS(=O)(=O)c3cnn(C)c3)n2)c(C)c1] | 1.16 | 549.216 | 550.5 | A |
| 295 | 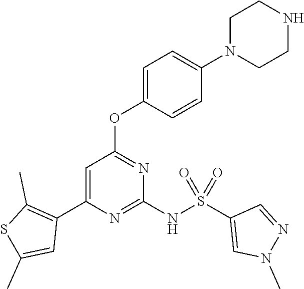 [SMILES: Cc1cc(-c2cc(Oc3ccc(N4CCNCC4)cc3)nc(NS(=O)(=O)c3cnn(C)c3)n2)c(C)s1] | 1.34 | 525.162 | 526.45 | A |
| 296 | 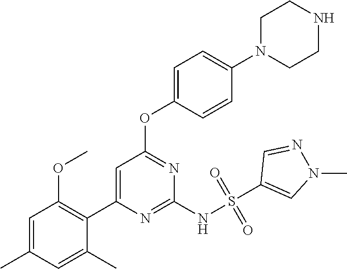 | 1.23 | 549.216 | 550.5 | A |
| 297 | 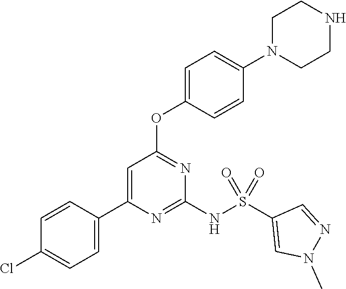 [SMILES: Cn1cc(S(=O)(=O)Nc2nc(Oc3ccc(N4CCNCC4)cc3)cc(-c3ccc(Cl)cc3)n2)cn1] | 1.34 | 525.135 | 526.41 | A |
| 298 | 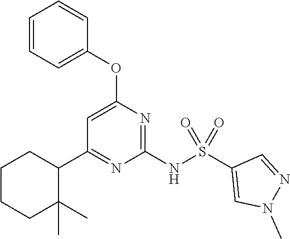 [SMILES: Cn1cc(S(=O)(=O)Nc2nc(Oc3ccccc3)cc(C3CCCCC3(C)C)n2)cn1] | 1.28 | 441.183 | 442.28 | R |
| 299 | 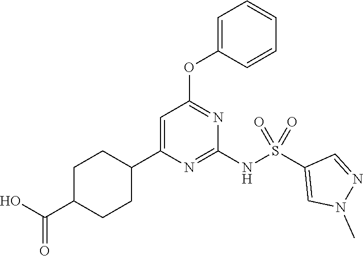 [SMILES: Cn1cc(S(=O)(=O)Nc2nc(Oc3ccccc3)cc(C3CCC(C(=O)O)CC3)n2)cn1] | 1.15 | 457.142 | 458.41 | A |
| 300 | 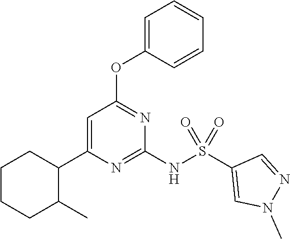 [SMILES: CC1CCCCC1c1cc(Oc2ccccc2)nc(NS(=O)(=O)c2cnn(C)c2)n1] | 1.68 | 427.168 | 428.17 | A |
| 301 | 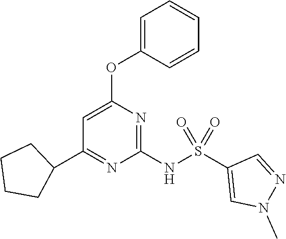 [SMILES: Cn1cc(S(=O)(=O)Nc2nc(Oc3ccccc3)cc(C3CCCC3)n2)cn1] | 1.43 | 399.137 | 399.98 | A |
| 302 | 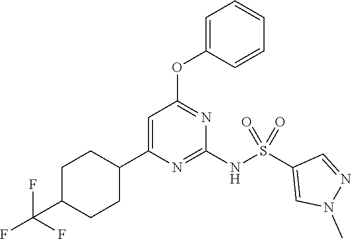 [SMILES: Cn1cc(S(=O)(=O)Nc2nc(Oc3ccccc3)cc(C3CCC(C(F)(F)F)CC3)n2)cn1] | 1.68 | 481.14 | 481.84 | A |
| 303 | 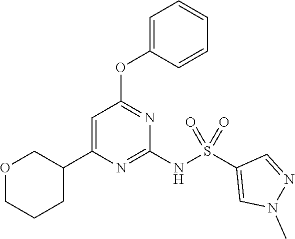 [SMILES: Cn1cc(S(=O)(=O)Nc2nc(Oc3ccccc3)cc(C3CCCOC3)n2)cn1] | 1.27 | 415.131 | 415.77 | A |
| 304 | 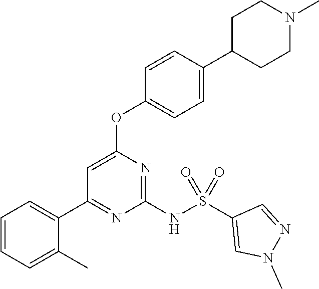 [SMILES: Cc1ccccc1-c1cc(Oc2ccc(C3CCN(C)CC3)cc2)nc(NS(=O)(=O)c2cnn(C)c2)n1] | 1.3 | 518.21 | 519.34 | A |
| 266 | 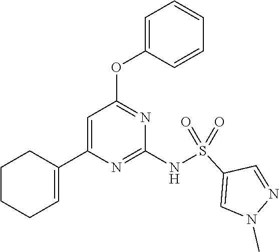 [SMILES: Cn1cc(S(=O)(=O)Nc2nc(Oc3ccccc3)cc(C3=CCCCC3)n2)cn1] | 1.85 | 411.137 | 412.4 | A |
| 305 | 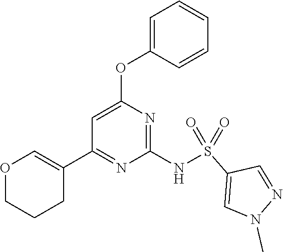 [SMILES: Cn1cc(S(=O)(=O)Nc2nc(Oc3ccccc3)cc(C3=COCCC3)n2)cn1] | 1.62 | 413.116 | 414.36 | A |
| 306 | 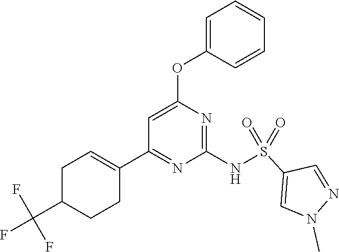 [SMILES: Cn1cc(S(=O)(=O)Nc2nc(Oc3ccccc3)cc(C3=CCC(C(F)(F)F)CC3)n2)cn1] | 1.94 | 479.124 | 480.44 | A |
| 307 | 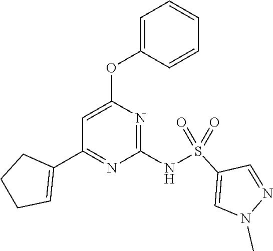 [SMILES: Cn1cc(S(=O)(=O)Nc2nc(Oc3ccccc3)cc(C3=CCCC3)n2)cn1] | 1.69 | 397.121 | 398.36 | A |
| 308 | 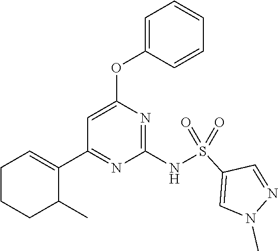 [SMILES: CC1CCCC=C1c1cc(Oc2ccccc2)nc(NS(=O)(=O)c2cnn(C)c2)n1] | 1.81 | 425.152 | 426.41 | A |
| 309 | 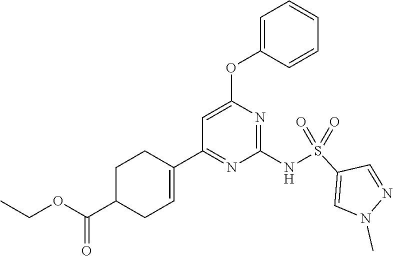 [SMILES: CCOC(=O)C1CC=C(c2cc(Oc3ccccc3)nc(NS(=O)(=O)c3cnn(C)c3)n2)CC1] | 1.65 | 483.158 | 484.46 | A |
| 310 | 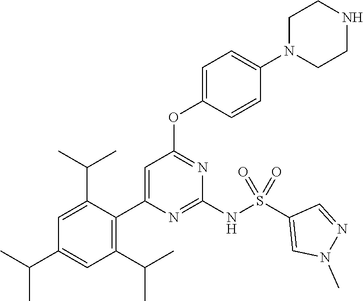 [SMILES: CC(C)c1cc(C(C)C)c(-c2cc(Oc3ccc(N4CCNCC4)cc3)nc(NS(=O)(=O)c3cnn(C)c3)n2)c(C(C)C)c1] | 1.74 | 617.315 | 618.67 | A |
| 311 | 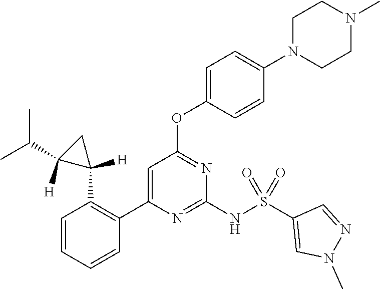 [SMILES: CC(C)[C@@H]1C[C@@H]1c1ccccc1-c1cc(Oc2ccc(N3CCN(C)CC3)cc2)nc(NS(=O)(=O)c2cnn(C)c2)n1] | 1.4 | 587.268 | 588.5 | A |

| Compound | |
|---|---|
| number | NMR |
| 278 | ¹H NMR (400 MHz, DMSO-d₆) δ 10.48 (s, 1H), 7.70 (s, 1H), 7.32 (s, 1H), 7.26- |
| | 7.17 (m, 3H), 7.17-7.11 (m, 2H), 7.01-6.91 (m, 2H), 6.71 (s, 1H), 4.00 (t, J = |
| | 6.7 Hz, 2H), 3.92-3.80 (m, 2H), 3.76 (s, 3H), 3.54-3.46 (m, 2H), 3.27-3.01 (m, |
| | 4H), 2.83 (d, J = 4.6 Hz, 3H), 2.25 (s, 3H), 1.77 (dq, J = 13.3, 6.7 Hz, 1H), 1.67- |
| | 1.54 (m, 2H), 0.93 (d, J = 6.6 Hz, 6H). |
| 279 | ¹H NMR (400 MHz, DMSO-d6) δ 10.51 (s, 1H), 7.79 (s, 1H), 7.68-7.60 (m, 2H), |
| | 7.50-7.37 (m, 2H), 7.28-7.04 (m, 6H), 4.03 (t, J = 6.5 Hz, 2H), 3.77 (s, 3H), |
| | 3.56-3.46 (m, 2H), 3.27-3.01 (m, 4H), 2.83 (d, J = 4.6 Hz, 3H), 1.81-1.61 (m, |
| | 2H), 1.37-1.30 (m, 2H), 0.91 (s, 9H). |

### Example 95: Preparation of Compound 312

### Step 1: N-[4-[2-(methoxymethyl)phenyl]-6-phenoxy-pyrimidin-2-yl]-3-nitro-benzenesulfonamide

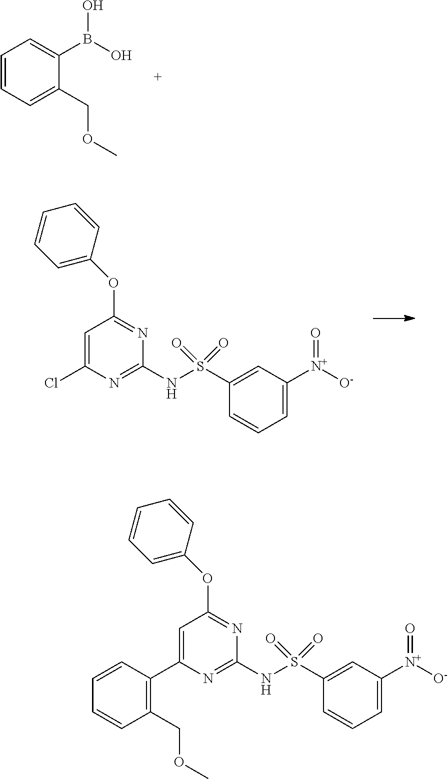 [SMILES: COCc1ccccc1B(O)O ; O=[N+]([O-])c1cccc(S(=O)(=O)Nc2nc(Cl)cc(Oc3ccccc3)n2)c1 ; COCc1ccccc1-c1cc(Oc2ccccc2)nc(NS(=O)(=O)c2cccc([N+](=O)[O-])c2)n1]

To a solution of N-(4-chloro-6-phenoxy-pyrimidin-2-yl)-3-nitro-benzenesulfonamide (100 mg, 0.2458 mmol), and [2-(methoxymethyl)phenyl]boronic acid (approximately 53.03 mg, 0.3195 mmol) in NMP (3 mL) was added Pd(dppf)Cl₂-DCM (approximately 200.7 mg, 0.2458 mmol) and potassium carbonate (approximately 491.6 μL of 2 M, 0.9832 mmol). The reaction mixture was flushed with nitrogen and it was heated at 100° C. for 60 minutes. It was filtered, and was purified by reverse phase HPLC using 10-99% acetonitrile in water to afford N-[4-[2-(methoxymethyl)phenyl]-6-phenoxy-pyrimidin-2-yl]-3-nitro-benzenesulfonamide (75 mg, 62%) ESI-MS m/z calc. 492.11035, found 493.46 (M+1)⁺; Retention time: 0.7 minutes; LC method D.

### Step 2: 3-amino-N-[4-[2-(methoxymethyl)phenyl]-6-phenoxy-pyrimidin-2-yl]benzenesulfonamide (Compound 312)

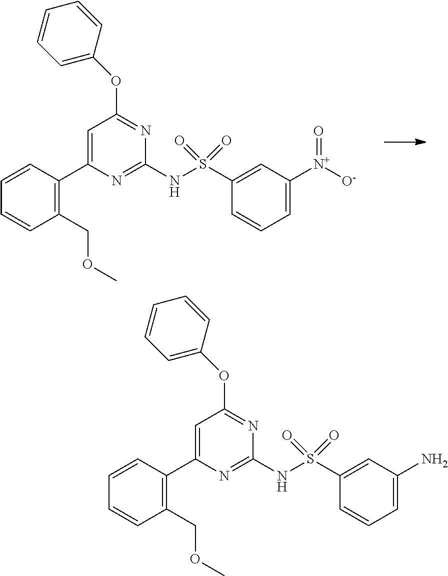

To a solution of N-[4-[2-(methoxymethyl)phenyl]-6-phenoxy-pyrimidin-2-yl]-3-nitro-benzenesulfonamide (10 mg, 0.02030 mmol) in MeOH (1 mL) was added palladium on carbon (approximately 11.50 mg of 10% w/w, 0.01081 mmol) and was stirred under H₂ atmosphere for 90 minutes. It was filtered and was purified by reverse phase HPLC using 1-99% acetonitrile in water using HCl as modifier to afford and 3-amino-N-[4-[2-(methoxymethyl)phenyl]-6-phenoxy-pyrimidin-2-yl]benzenesulfonamide (9.8 mg, 98%). ESI-MS m/z calc. 462.13617, found 463.52 (M+1)⁺; Retention time: 1.58 minutes; LC method A.

### Example 96: Preparation of Compound 313

### Step 1: N-[4-[2-(methoxymethyl)phenyl]-6-(2-methylphenoxy)pyrimidin-2-yl]-3-nitro-benzenesulfonamide

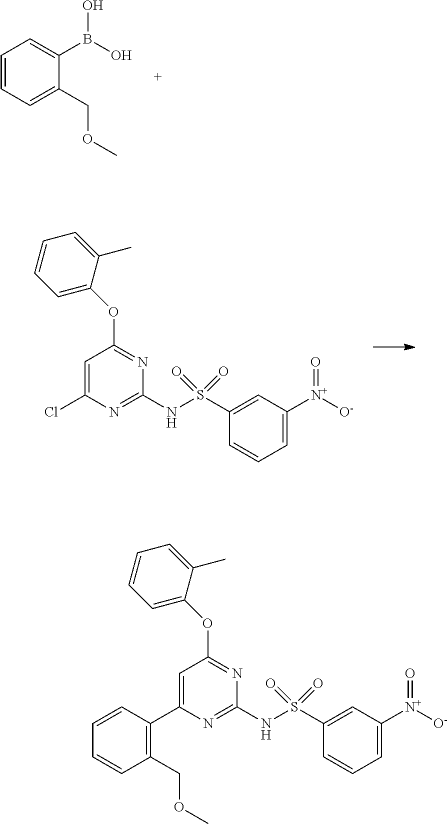 [SMILES: COCc1ccccc1B(O)O ; Cc1ccccc1Oc1cc(Cl)nc(NS(=O)(=O)c2cccc([N+](=O)[O-])c2)n1 ; COCc1ccccc1-c1cc(Oc2ccccc2C)nc(NS(=O)(=O)c2cccc([N+](=O)[O-])c2)n1]

To a solution of N-[4-chloro-6-(2-methylphenoxy)pyrimidin-2-yl]-3-nitro-benzenesulfonamide (100 mg, 0.2376 mmol) and [2-(methoxymethyl)phenyl]boronic acid (approximately 39.44 mg, 0.2376 mmol) in NMP (3 mL) was added potassium carbonate (approximately 365.6 μL of 2 M, 0.7311 mmol) followed by Pd(dppf)C₁₂ (approximately 149.3 mg, 0.1828 mmol). The reaction mixture was flushed with nitrogen and it was heated in a sealed tube at 100° C. for 1 hour. It was filtered and the material was purified by reverse phase HPLC using 10-99% acetonitrile in water to afford N-[4-[2-(methoxymethyl)phenyl]-6-(2-methylphenoxy)pyrimidin-2-yl]-3-nitro-benzenesulfonamide (65 mg, 70%) ESI-MS m/z calc. 506.126, found 507.5 (M+1)⁺; Retention time: 0.73 minutes; LC method D.

### Step 3: 3-amino-N-[4-[2-(methoxymethyl)phenyl]-6-(2-methylphenoxy)pyrimidin-2-yl]benzenesulfonamide (Compound 313)

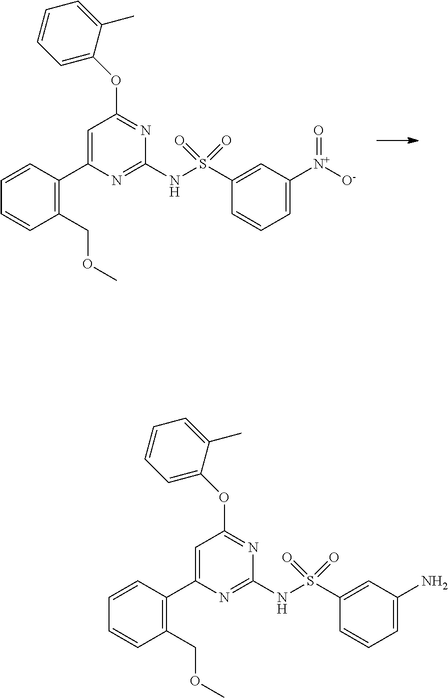 [SMILES: COCc1ccccc1-c1cc(Oc2ccccc2C)nc(NS(=O)(=O)c2cccc([N+](=O)[O-])c2)n1 ; COCc1ccccc1-c1cc(Oc2ccccc2C)nc(NS(=O)(=O)c2cccc(N)c2)n1]

To a solution of N-[4-[2-(methoxymethyl)phenyl]-6-(2-methylphenoxy)pyrimidin-2-yl]-3-nitro-benzenesulfonamide (25 mg, 0.04936 mmol) in MeOH (1 mL) was added Pd (12 mg of 10% w/w, 0.01128 mmol) and stirred under H₂ for 90 minutes. The reaction mixture was filtered and was purified by reverse phase HPLC using 1-99% acetonitrile in water using HCl as a modifier to afford 3-amino-N-[4-[2-(methoxymethyl)phenyl]-6-(2-methylphenoxy)pyrimidin-2-yl]benzenesulfonamide (7 mg, 25%) ESI-MS m/z calc. 476.15182, found 477.53 (M+1)⁺; Retention time: 1.7 minutes; LC method A.

### Example 97: Preparation of Compound 314

### Step 1: N-[4-chloro-6-(2-chloro-6-methyl-phenoxy)pyrimidin-2-yl]-3-nitro-benzenesulfonamide

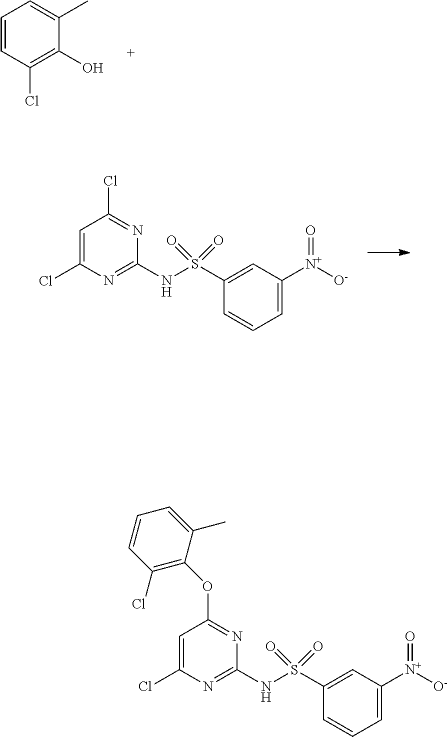

To a mixture of N-(4,6-dichloropyrimidin-2-yl)-3-nitro-benzenesulfonamide (500 mg, 1.432 mmol), 2-chloro-6-methyl-phenol (approximately 204.2 mg, 1.432 mmol) and K₂CO₃ (approximately 593.7 mg, 4.296 mmol) was added DMSO (5 mL) and the mixture was heated at 100° C. for 4 hours. To this reaction mixture was added water and then acidified with 2 N HCl at which point the product crashed out. The solid was filtered and the precipitate was again purified by reverse phase HPLC using 10-99% acetonitrile in water using HCl as modifier to afford N-[4-chloro-6-(2-chloro-6-methyl-phenoxy)pyrimidin-2-yl]-3-nitro-benzenesulfonamide (341 mg, 52%) ESI-MS m/z calc. 453.99054, found 455.37 (M+1)⁺; Retention time: 0.75 minutes; LC method D.

### Step 2: N-[4-(2-chloro-6-methyl-phenoxy)-6-(o-tolyl)pyrimidin-2-yl]-3-nitro-benzenesulfonamide

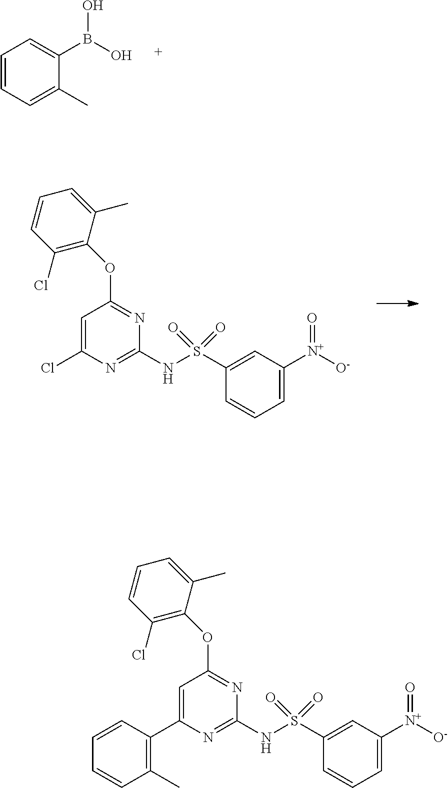

To a solution of N-[4-chloro-6-(2-chloro-6-methyl-phenoxy)pyrimidin-2-yl]-3-nitro-benzenesulfonamide (100 mg, 0.2196 mmol) and o-tolylboronic acid (approximately 26.86 mg, 0.1976 mmol) in NMP (3 mL) was added potassium carbonate (approximately 439.2 μL of 2 M, 0.8784 mmol) and Pd(dppf)C₁₂ (approximately 35.87 mg, 0.04392 mmol). The reaction mixture was flushed with nitrogen and it was heated in a sealed tube at 100° C. for 2 hours. The reaction was filtered and was purified by reverse phase HPLC using 10-99% acetonitrile in water to afford N-[4-(2-chloro-6-methyl-phenoxy)-6-(o-tolyl)pyrimidin-2-yl]-3-nitro-benzenesulfonamide (48 mg, 43%) ESI-MS m/z calc. 510.07648, found 511.46 (M+1)⁺; Retention time: 0.79 minutes; LC method D.

### Step 3: 3-amino-N-[4-(2-chloro-6-methyl-phenoxy)-6-(o-tolyl)pyrimidin-2-yl]benzenesulfonamide (Compound 314)

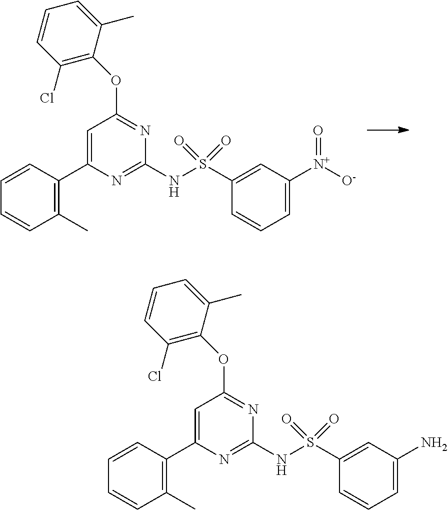 [SMILES: Cc1ccccc1-c1cc(Oc2c(C)cccc2Cl)nc(NS(=O)(=O)c2cccc([N+](=O)[O-])c2)n1 ; Cc1ccccc1-c1cc(Oc2c(C)cccc2Cl)nc(NS(=O)(=O)c2cccc(N)c2)n1]

To a solution of N-[4-(2-chloro-6-methyl-phenoxy)-6-(o-tolyl)pyrimidin-2-yl]-3-nitro-benzenesulfonamide (20 mg, 0.03914 mmol) or N-[4-(2-chloro-6-methyl-phenoxy)-6-[2-(methoxymethyl)phenyl]pyrimidin-2-yl]-3-nitro-benzenesulfonamide (20 mg, 0.03697 mmol) in AcOH (1 mL) was added zinc (approximately 12.80 mg, 1.794 μL, 0.1957 mmol). The reaction mixture was heated at 60° C. for 30 minutes. The reaction was filtered, and the resulting solution was evaporated under reduced pressure. The residue was dissolved in DMSO and was purified by reverse phase HPLC using 1-99% acetonitrile in water using HCl as modifier to afford 3-amino-N-[4-(2-chloro-6-methyl-phenoxy)-6-(o-tolyl)pyrimidin-2-yl]benzenesulfonamide (4.2 mg, 22%) ESI-MS m/z calc. 480.1023, found 481.45 (M+1)⁺; Retention time: 1.87 minutes; LC method A.

### Example 98: Preparation of Compound 315

### Step 1: N-[4-(2-chloro-6-methyl-phenoxy)-6-[2-(methoxymethyl)phenyl]pyrimidin-2-yl]-3-nitro-benzenesulfonamide

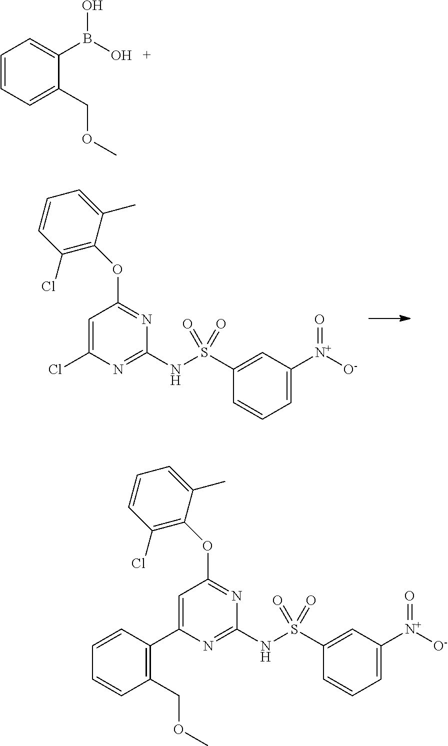 [SMILES: COCc1ccccc1B(O)O ; Cc1cccc(Cl)c1Oc1cc(Cl)nc(NS(=O)(=O)c2cccc([N+](=O)[O-])c2)n1 ; COCc1ccccc1-c1cc(Oc2c(C)cccc2Cl)nc(NS(=O)(=O)c2cccc([N+](=O)[O-])c2)n1]

To a solution of N-[4-chloro-6-(2-chloro-6-methyl-phenoxy)pyrimidin-2-yl]-3-nitro-benzenesulfonamide (100 mg, 0.2196 mmol) and [2-(methoxymethyl)phenyl]boronic acid (approximately 32.80 mg, 0.1976 mmol) in NMP (3 mL) was added potassium carbonate (approximately 439.2 μL of 2 M, 0.8784 mmol) and Pd(dppf)C₁₂ (approximately 35.87 mg, 0.04392 mmol). The reaction mixture was flushed with nitrogen and the mixture was heated in a sealed tube at 100° C. for 2 hours. The solution was filtered and was purified by reverse phase HPLC using 10-99% acetonitrile in water to afford N-[4-(2-chloro-6-methyl-phenoxy)-6-[2-(methoxymethyl)phenyl]pyrimidin-2-yl]-3-nitro-benzenesulfonamide (61 mg, 51%) ESI-MS m z calc. 540.08704, found 541.47 (M+1)⁺; Retention time: 0.76 minutes; LC method D.

### Step 2: 3-Amino-N-[4-(2-chloro-6-methyl-phenoxy)-6-[2-(methoxymethyl)phenyl]pyrimidin-2-yl]benzenesulfonamide (Compound 315)

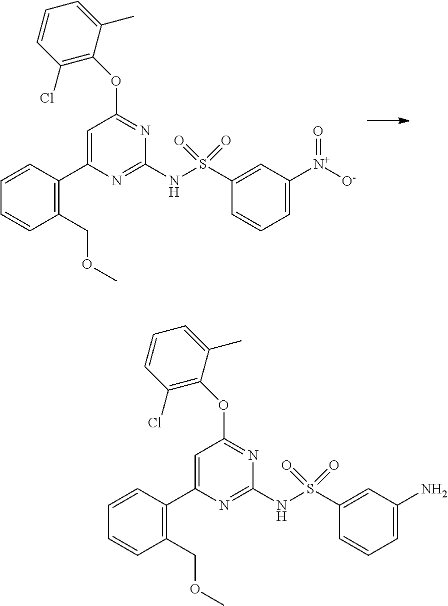 [SMILES: COCc1ccccc1-c1cc(Oc2c(C)cccc2Cl)nc(NS(=O)(=O)c2cccc([N+](=O)[O-])c2)n1 ; COCc1ccccc1-c1cc(Oc2c(C)cccc2Cl)nc(NS(=O)(=O)c2cccc(N)c2)n1]

To a solution N-[4-(2-chloro-6-methyl-phenoxy)-6-[2-(methoxymethyl)phenyl]pyrimidin-2-yl]-3-nitro-benzenesulfonamide (20 mg, 0.03697 mmol) in AcOH (1 mL) was added zinc (approximately 12.80 mg, 1.794 μL, 0.1957 mmol). The reaction mixture was heated at 60° C. for 30 minutes. It was filtered, the acetic acid was evaporated and the residue was dissolved in DMSO and was purified by reverse phase HPLC using 1-99% acetonitrile in water using HCl as modifier to afford 3-amino-N-[4-(2-chloro-6-methyl-phenoxy)-6-[2-(methoxymethyl)phenyl]pyrimidin-2-yl]benzenesulfonamide (3.4 mg, 18%) ESI-MS m/z calc. 510.11285, found 511.5 (M+1)⁺; Retention time: 1.78 minutes; LC method A.

### Example 99: Preparation of Compound 316

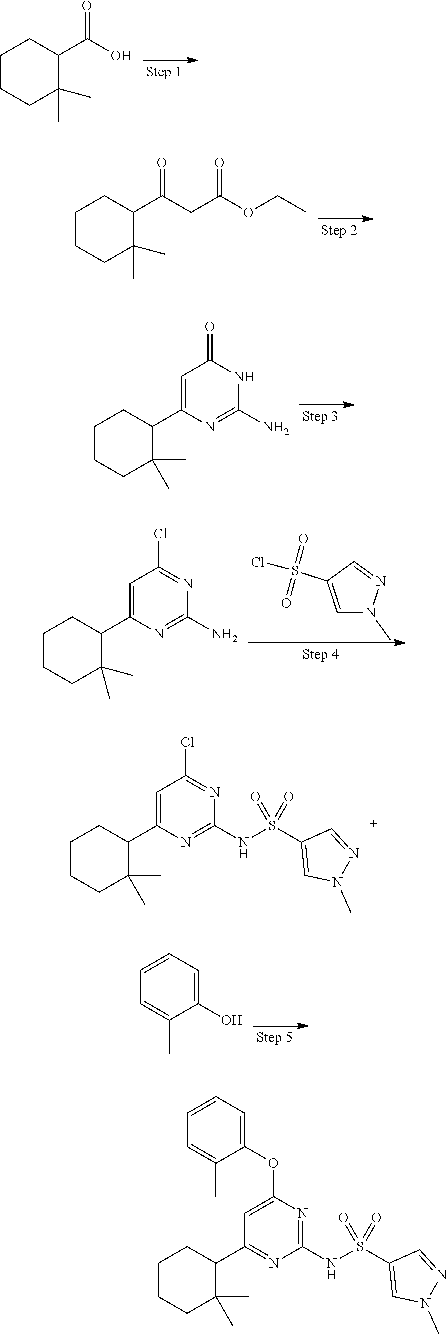

### Step 1: Ethyl 3-(2,2-Dimethylcyclohexyl)-3-oxo-propanoate

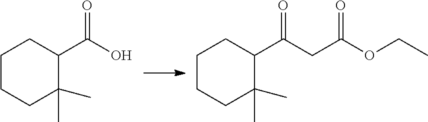 [SMILES: CC1(C)CCCCC1C(=O)O ; CCOC(=O)CC(=O)C1CCCCC1(C)C]

Stage 1: To a solution of 2,2-dimethylcyclohexanecarboxylic acid (5 g, 32.01 mmol) and DMF (approximately 117.0 mg, 123.9 μL, 1.600 mmol) in dichloromethane/hexanes (1:1, 128.0 mL) at 0° C. was slowly added oxalyl chloride (approximately 24.38 g, 16.76 mL, 192.1 mmol). The reaction was stirred for 1 hour until bubbling ceased. The reaction mixture was concentrated and placed under vacuum.

Stage 2: To a solution of LDA (approximately 32.33 mL of 2 M, 64.66 mmol) at −78° C. was added dropwise ethyl acetate (approximately 5.725 g, 6.347 mL, 64.98 mmol). After 10 minutes, a solution of the acid chloride from stage 1 dissolved in THE (32 mL) was added dropwise. The reaction was allowed to warm to 23° C. and then was quenched with acetic acid (approximately 2.883 g, 2.730 mL, 48.01 mmol). Water was added and the aqueous layer was extracted with ethyl acetate (3×). The combined organics were dried with brine, magnesium sulfate, filtered and concentrated under reduced pressure. The crude residue was purified by flash column chromatography on silica gel (0 to 100% Ethyl acetate in hexanes). ethyl 3-(2,2-dimethylcyclohexyl)-3-oxo-propanoate (6.29 g, 87%) was isolated as a light yellow oil. ESI-MS m/z calc. 226.15689, found 227.22 (M+1)⁺; Retention time: 0.85 minutes; LC method D.

### Step 2: 2-Amino-4-(2,2-dimethylcyclohexyl)-1H-pyrimidin-6-one

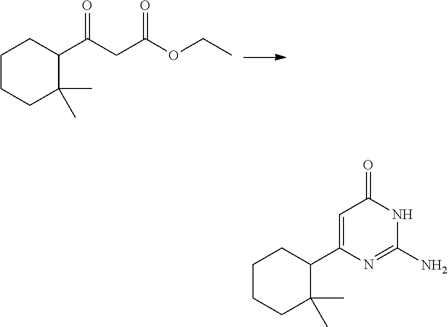 [SMILES: CCOC(=O)CC(=O)C1CCCCC1(C)C ; CC1(C)CCCCC1c1cc(=O)[nH]c(N)n1]

To a solution of ethyl 3-(2,2-dimethylcyclohexyl)-3-oxo-propanoate (6.29 g, 27.79 mmol) and guanidine (hydrochloride salt) (approximately 3.186 g, 33.35 mmol) in methanol (55.45 mL) at 23° C. was added potassium tert-butoxide (approximately 16.84 g, 150.1 mmol) portion wise. The reaction was heated to 85° C. for 12 hours in a pressure vessel. The reaction was cooled to 23° C. and 12 mL of acetic acid was added and then the mixture was further diluted with 50 mL of methanol. The crude mixture was concentrated on to silica gel. The product was purifie by flash column chromatography on silica gel (10% methanol in dichloromethane). 2-amino-4-(2,2-dimethylcyclohexyl)-1H-pyrimidin-6-one (3.52 g, 57%) was isolated as a yellow solid. ESI-MS m/z calc. 221.15282, found 222.25 (M+1)⁺; Retention time: 0.36 minutes; LC method D.

### Step 3: 4-Chloro-6-(2,2-dimethylcyclohexyl)pyrimidin-2-amine

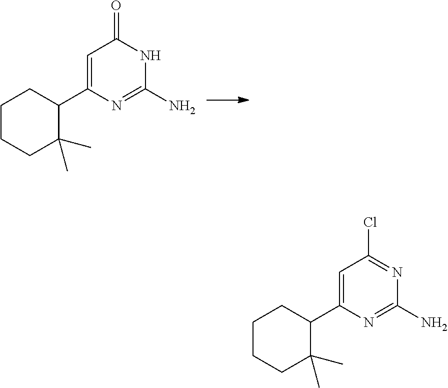 [SMILES: CC1(C)CCCCC1c1cc(=O)[nH]c(N)n1 ; CC1(C)CCCCC1c1cc(Cl)nc(N)n1]

2-Amino-4-(2,2-dimethylcyclohexyl)-1H-pyrimidin-6-one (3.52 g, 15.91 mmol) was dissolved in POCl₃ (approximately 29.27 g, 17.79 mL, 190.9 mmol) and the resulting solution was heated to 95° C. for 4 hours. The excess POCl₃ was removed in vacuo. The crude residue was dissolved in dichloromethane and a saturated aqueous solution of sodium bicarbonate was added. The biphasic mixture was stirred rapidly for 20 minutes. The organic layer was removed, and the aqueous layer was further extracted with dichloromethane (4×). The combined organic layers were dried over sodium sulfate, filtered, and concentrated in vacuo. The crude residue was purified by flash column chromatography on silica gel (20% ethyl acetate in hexanes). 4-Chloro-6-(2,2-dimethylcyclohexyl)pyrimidin-2-amine (1.91 g, 40%) was isolated as a white solid. ESI-MS m/z calc. 239.11893, found 240.22 (M+1)⁺; Retention time: 0.62 minutes; LC method D.

### Step 4: N-[4-Chloro-6-(2,2-dimethylcyclohexyl)pyrimidin-2-yl]-1-methyl-pyrazole-4-sulfonamide

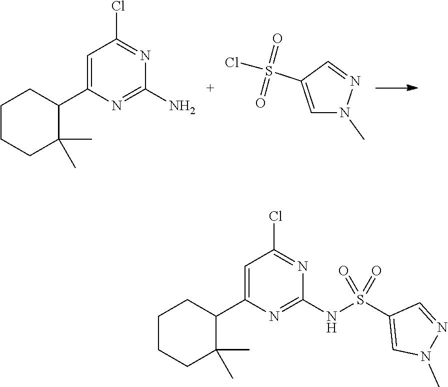

To a solution of 4-chloro-6-(2,2-dimethylcyclohexyl)pyrimidin-2-amine (900 mg, 3.754 mmol) in DMF (15.40 mL) at 0° C. was added NaH (approximately 360.4 mg, 15.02 mmol) and the reaction mixture was stirred at this temperature for 5 minutes, then removed from the cooling bath and stirred at room temperature for 10 minutes. The reaction mixture was cooled to 0° C. and a solution of 1-methylpyrazole-4-sulfonyl chloride (approximately 1.356 g, 7.508 mmol) in DMF (3.0 mL) was added dropwise over 1 minute. The reaction mixture was stirred at this temperature for 5 minutes, then removed from the cooling bath and stirred at room temperature for 12 minutes. The reaction mixture was cooled back to 0° C. and quenched with HCl (approximately 1.283 mL of 48% w/v, 16.89 mmol), then diluted with a solution of ethyl acetate/hexanes (1:1) and water. A saturated aqueous solution of sodium bicarbonate was added until the heterogeneous mixture was completely dissolved, and the acidic solution was neutralized. The organic layer was removed, and the aqueous layer was further extracted with ethyl acetate/hexanes (1:1, 5×). The combined organic extracts were dried with brine and magnesium sulfate. The solution was filtered, and the filtrate was concentrated in vacuo. The crude residue was purified by flash column chromatography on silica gel (5 to 40% ethyl acetate in hexanes). N-[4-chloro-6-(2,2-dimethylcyclohexyl)pyrimidin-2-yl]-1-methyl-pyrazole-4-sulfonamide (1.00 g, 69%) was isolated as a white solid. ESI-MS m/z calc. 383.1183, found 384.32 (M+1)⁺; Retention time: 0.67 minutes; LC method D.

### Step 5: N-[4-(2,2-Dimethylcyclohexyl)-6-(2-methylphenoxy)pyrimidin-2-yl]-1 -methyl-pyrazole-4-sulfonamide (Compound 316)

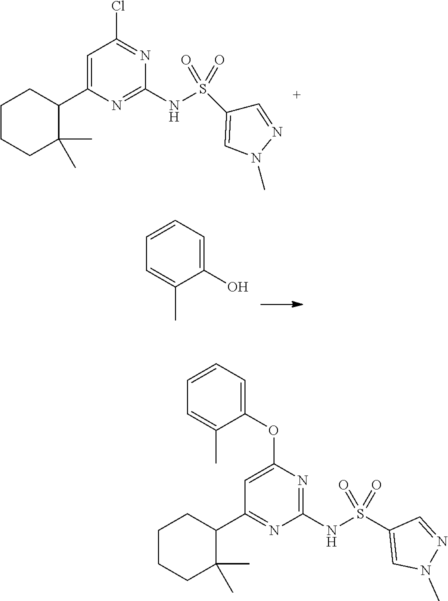

A heterogenous solution of o-cresol (approximately 17.17 mg, 31.07 μL, 0.1588 mmol), and cesium carbonate in NMP (400 μL) was sealed in a vial and the reaction was heated at 110° C. for 16 hours. The reaction was cooled and diluted with DMSO, filtered, and submitted to purification by reverse phase chromatography (acetonitrile in water with 0.1% hydrochloric acid) to give N-[4-(2,2-dimethylcyclohexyl)-6-(2-methylphenoxy)pyrimidin-2-yl]-1-methyl-pyrazole-4-sulfonamide (17.5 mg, 69%). ESI-MS m/z calc. 455.1991, found 456.32 (M+1)⁺; Retention time: 1.34 minutes; LC method E.

### Example 100: Preparation of Compound 317

### Step 1: 2-amino-6-cyclohexyl-pyrimidin-4-ol

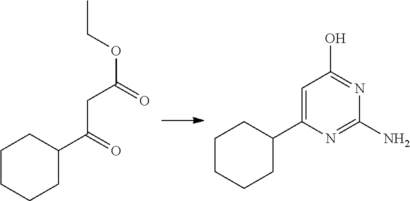

To a stirring solution of ethyl 3-cyclohexyl-3-oxo-propanoate (4.81 g, 24.26 mmol) and guanidine (hydrochloride salt) (approximately 2.781 g, 29.11 mmol) in methanol (56.42 mL) at room temperature, was added potassium tert-butoxide (Potassium Ion (1)) (approximately 14.70 g, 131.0 mmol) in 5 portions. The reaction mixture was then heated to 60° C. for 45 minutes. After this time the reaction mixture was cooled to room temperature and allowed to stir over the weekend (64 hours). The reaction mixture was filtered to remove the resulting salts, and the filtrate was concentrated to about 10 mL. After dilution with 10 mL of water the resulting solution was cooled in an ice bath and acidified to pH 5 with 6 M HCl, resulting in a slightly yellow precipitate, which was collected by filtration and dried on a high vac. 2-amino-6-cyclohexyl-pyrimidin-4-ol (3.671 g, 78%). ¹H NMR (400 MHz, DMSO) δ 10.56 (s, 1H), 6.41 (s, 2H), 5.34 (s, 1H), 2.13 (t, J=11.2 Hz, 1H), 1.73 (d, J=11.0 Hz, 4H), 1.65 (d, J=12.6 Hz, 1H), 1.39-1.11 (m, 5H). ESI-MS m/z calc. 193.1215, found 194.1 (M+1)⁺; Retention time: 0.29 minutes; LC method D.

### Step 2: 4-chloro-6-cyclohexyl-pyrimidin-2-amine

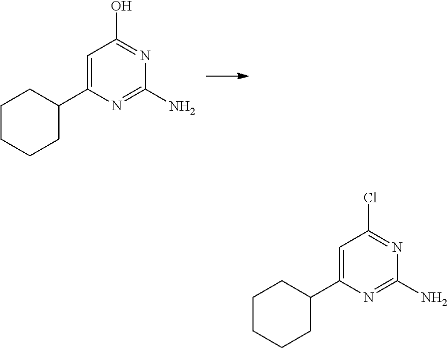 [SMILES: Nc1nc(O)cc(C2CCCCC2)n1 ; Nc1nc(Cl)cc(C2CCCCC2)n1]

2-Amino-6-cyclohexyl-pyrimidin-4-ol (1.434 g, 7.421 mmol) and phosphorus oxychloride (6.25 mL, 67.05 mmol) were combined in a screwcap vial and heated to 100° C. for 2 hours. After cooling to room temperature, the volatiles were removed under reduced pressure, and the remaining residue was diluted with dichloromethane, cooled in an ice bath, and carefully neutralized by the addition of saturated aqueous sodium bicarbonate to pH=8. The resulting biphasic mixture was stirred for an hour at room temperature, then poured into a separatory funnel. The organics were separated, and the aqueous portion was extracted an additional 2×50 mL dichloromethane. The combined organics were washed with water then brine, and dried over sodium sulfate, before being concentrated to give a slightly yellow foam, which was used in the enxt step without further purification. 4-chloro-6-cyclohexyl-pyrimidin-2-amine (1.169 g, 74%) ESI-MS m/z calc. 211.08763, found 212.1 (M+1)⁺; Retention time: 0.53 minutes; LC method D.

### Step 3: N-(4-chloro-6-cyclohexyl-pyrimidin-2-yl)-1-methyl-pyrazole-4-sulfonamide

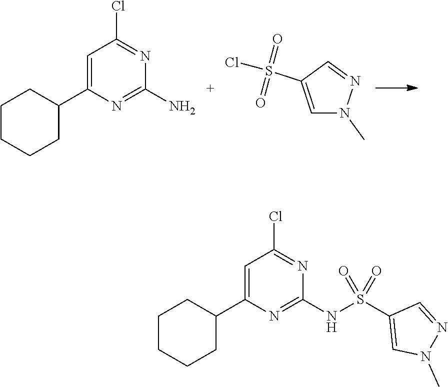

To a solution of 4-chloro-6-cyclohexyl-pyrimidin-2-amine (780 mg, 3.685 mmol) in DMF (12.28 mL), cooled in an ice bath, was added sodium hydride (750 mg, 18.75 mmol) in several portions. After 5 minutes, the reaction mixture was removed from the ice bath and allowed to stir at room temperature for an additional 20 minutes. The reaction mixture was then again cooled in the ice bath, and 1-methylpyrazole-4-sulfonyl chloride (1,000 mg, 5.537 mmol) was added in a single portion. After 5 minutes, the reaction mixture was allowed to warm to room temperature and stirred for an additional 30 minutes. The reaction mixture was then slowly poured into chilled water (50 mL) and acidified with 1 N HCl to pH 4. The resulting precipitate was collected by vacuum filtration, washing with water, and dried on high vacuum to give a beige solid. N-(4-chloro-6-cyclohexyl-pyrimidin-2-yl)-1-methyl-pyrazole-4-sulfonamide (1.310 g, 70%). ESI-MS m/z calc. 355.08698, found 356.1 (M+1)⁺; Retention time: 0.59 minutes; LC method D.

### Step 4: N-[4-cyclohexyl-6-(2-methylphenoxy)pyrimidin-2-yl]-1-methyl-pyrazole-4-sulfonamide (Compound 317)

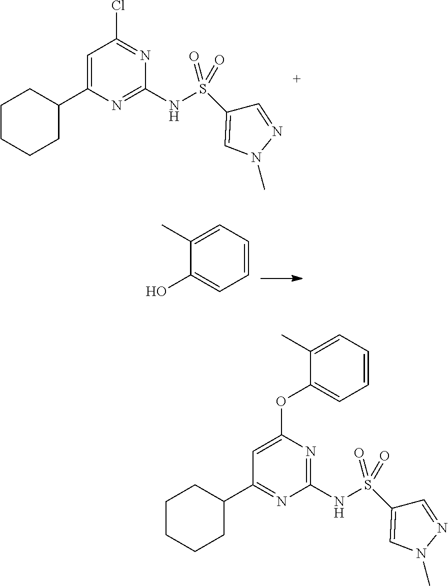

N-(4-Chloro-6-cyclohexyl-pyrimidin-2-yl)-1-methyl-pyrazole-4-sulfonamide (30 mg, 0.08431 mmol), Cs₂CO₃ (approximately 137.4 mg, 0.4216 mmol), and o-cresol (approximately 36.46 mg, 65.97 μL, 0.3372 mmol) were combined in anhydrous NMP (0.4 mL). The reaction was heated to 100° C. Upon completion, the reaction mixture was filtered, diluted with methanol to a 0.8 mL volume and purified by Prep HPLC (0-99% MeCN), to give N-[4-cyclohexyl-6-(2-methylphenoxy)pyrimidin-2-yl]-1-methyl-pyrazole-4-sulfonamide (8.5 mg, 25%). ESI-MS m z calc. 427.16782, found 428.2 (M+1)⁺; Retention time: 1.65 minutes; LC method A.

### Example 101: Preparation of Compound 318

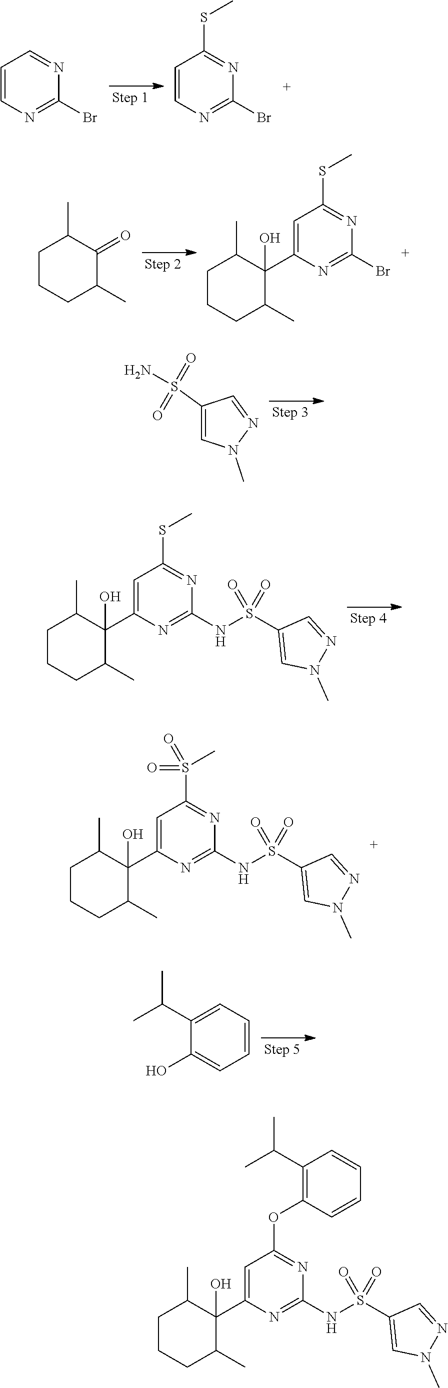

### Step 1: 2-bromo-4-methylsulfanyl-pyrimidine

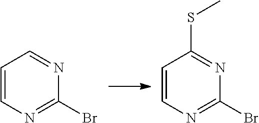

To a heterogeneous solution of 2-bromopyrimidine (approximately 8.332 g, 52.41 mmol) in THE (52.41 mL) at −51° C. (acetonitrile/dry ice bath) was added dropwise lithium;chloro-(2,2,6,6-tetramethyl-1-piperidyl)magnesium;chloride (approximately 57.65 mL of 1 M, 57.65 mmol). The resultant solution was stirred for 1.5 hours before adding methylsulfonylsulfanylmethane (9.92 g, 78.61 mmol) slowly over 5 minutes. The solution was then allowed to warm to room temperature. Acetic acid (7.0 mL), saturated aqueous sodium bicarbonate and diethyl ether were added in succession. The biphasic mixture was filtered, and the organic layer removed. The aqueous layer was further extracted with diethyl ether (3×). The combined organic layers were dried with brine, magnesium sulfate, filtered, and then concentrated in vacuo onto silica gel. The crude residue was separated by flash column chromatography on silica gel (20% ethyl acetate in hexanes). 2-Bromo-4-methylsulfanyl-pyrimidine (490 mg, 4%) was isolated as a white solid. ESI-MS m/z calc. 203.93568, found 205.05 (M+1)⁺; Retention time: 0.43 minutes; LC method D.

### Step 2: 1-(2-bromo-6-methylsulfanyl-pyrimidin-4-yl)-2,6-dimethyl-cyclohexanol

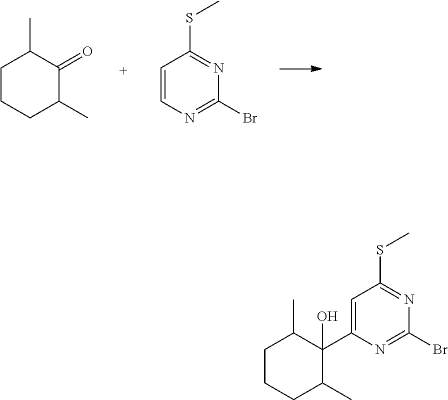

To a solution of 2-bromo-4-methylsulfanyl-pyrimidine (490 mg, 2.342 mmol) in THE (2.342 mL) at −51° C. was added lithium;chloro-(2,2,6,6-tetramethyl-1-piperidyl)magnesium;chloride (approximately 2.576 mL of 1 M, 2.576 mmol) dropwise. The reaction was allowed to stir at this temperature for 1.5 hours before slowly adding 2,6-dimethylcyclohexanone (approximately 384.3 mg, 3.045 mmol). The reaction was allowed to warm to 23° C. over 1 hour and was quenched with a saturated aqueous solution of sodium bicarbonate and then diluted with diethyl ether. The organic layer was removed, and the aqueous layer was further extracted with diethyl ether. The combined organic layers were concentrated in vacuo. The crude residue was separated by flash column chromatography on silica gel (gradient: 1 to 10% ethyl acetate in hexanes). 1-(2-bromo-6-methylsulfanyl-pyrimidin-4-yl)-2,6-dimethyl-cyclohexanol (250 mg, 32%) was isolated as a clear oil. ESI-MS m/z calc. 330.04016, found 331.3 (M+1)⁺; Retention time: 0.77 minutes; LC method A.

### Step 3: N-[4-(1-Hydroxy-2,6-dimethyl-cyclohexyl)-6-methylsulfanyl-pyrimidin-2-yl]-1-methyl-pyrazole-4-sulfonamide

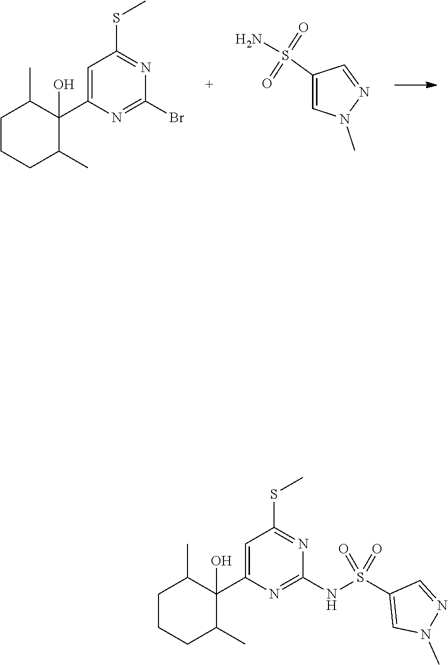

A heterogeneous solution consisting of [2-(2-aminoethyl)phenyl]-chloro-palladium;ditert-butyl-[2-(2,4,6-triisopropylphenyl)phenyl]phosphane (XPhos Pd G1)(approximately 51.82 mg, 0.07547 mmol), 1-(2-bromo-6-methylsulfanyl-pyrimidin-4-yl)-2,6-dimethyl-cyclohexanol (250 mg, 0.7547 mmol), 1-methylpyrazole-4-sulfonamide (approximately 182.5 mg, 1.132 mmol), and cesium carbonate (approximately 737.7 mg, 2.264 mmol) in dioxane (3.019 mL) was sealed in a vial and heated to 100° C. for 1 hour. The reaction was cooled, and the solvent removed in vacuo. The crude residue was partitioned between aqueous 1 N HCl and dichloromethane. The organic layer was removed, and the aqueous layer was further extracted with dichloromethane (4×). The combined organic extracts were dried over magnesium sulfate, filtered, and concentrated in vacuo. The crude residue was separated by flash column chromatography on silica gel (gradient: 5 to 95% ethyl acetate in hexanes). N-[4-(1-hydroxy-2,6-dimethyl-cyclohexyl)-6-methylsulfanyl-pyrimidin-2-yl]-1-methyl-pyrazole-4-sulfonamide (230 mg, 56%) was isolated as a clear oil (purity: 75%). ESI-MS m/z calc. 411.1399, found 412.37 (M+1)⁺; Retention time: 0.58 minutes; LC method D.

### Step 4: N-[4-(1-Hydroxy-2,6-dimethyl-cyclohexyl)-6-methylsulfonyl-pyrimidin-2-yl]-1-methyl-pyrazole-4-sulfonamide

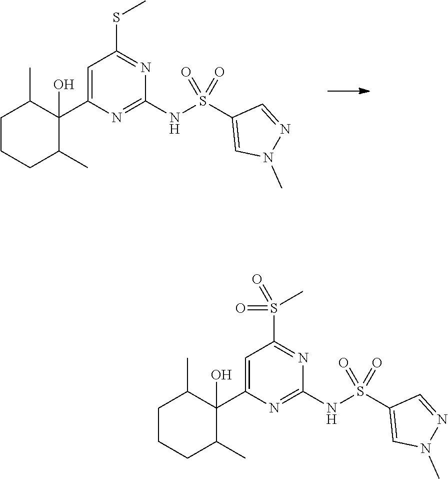 [SMILES: CSc1cc(C2(O)C(C)CCCC2C)nc(NS(=O)(=O)c2cnn(C)c2)n1 ; CC1CCCC(C)C1(O)c1cc(S(C)(=O)=O)nc(NS(=O)(=O)c2cnn(C)c2)n1]

To a solution of N-[4-(1-hydroxy-2,6-dimethyl-cyclohexyl)-6-methylsulfanyl-pyrimidin-2-yl]-1-methyl-pyrazole-4-sulfonamide (260 mg, 0.4738 mmol) in DCM (3.790 mL) was added m-CPBA (approximately 276.1 mg, 1.232 mmol). The reaction was stirred for 2 h, after which more m-CPBA (approximately 106.2 mg, 0.4738 mmol) was added. After 1 h, the reaction mixture was concentrated in vacuo onto silica gel. The silical gel was subjected to flash column chromatography (gradient: 0 to 100% ethyl acetate in hexanes). N-[4-(1-hydroxy-2,6-dimethyl-cyclohexyl)-6-methylsulfonyl-pyrimidin-2-yl]-1-methyl-pyrazole-4-sulfonamide (169mg, 75%) was isolated as a white foam (93% purity by UV−VIS). ESI-MS m/z calc. 443.1297, found 444.34 (M+1)⁺; Retention time: 0.5 minutes; LC method D.

### Step 5: N-[4-(1-hydroxy-2,6-dimethyl-cyclohexyl)-6-(2-isopropylphenoxy)pyrimidin-2-yl]-1-methyl-pyrazole-4-sulfonamide (Compound 318)

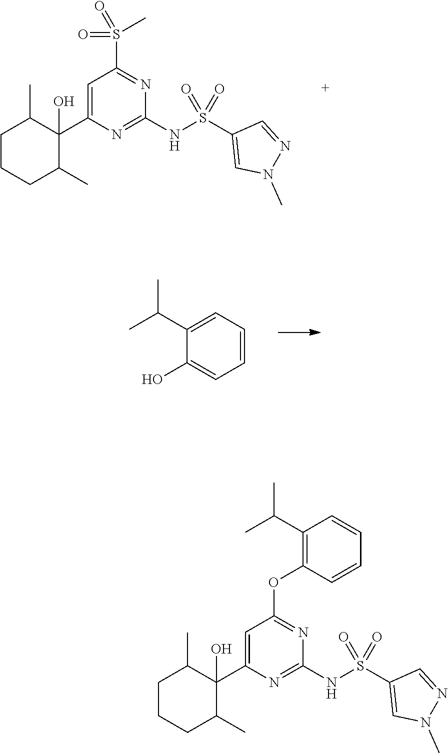

A heterogeneous mixture of 2-isopropylphenol (approximately 21.42 mg, 0.1573 mmol), N-[4-(1-hydroxy-2,6-dimethyl-cyclohexyl)-6-methylsulfonyl-pyrimidin-2-yl]-1-methyl-pyrazole-4-sulfonamide (25 mg, 0.05242 mmol), and cesium carbonate (approximately 68.32 mg, 0.2097 mmol) in NMP (209.7 μL) (0.30 mL) was heated to 120° C. in a sealed vial for 16 hours. To each vial was added acetic acid (0.10 mL) and DMSO (0.50 mL). The crude mixture was separated by reverse phase chromatography (acetonitrile in water with 0.1% hydrochloric acid). N-[4-(1-hydroxy-2,6-dimethyl-cyclohexyl)-6-(2-isopropylphenoxy)pyrimidin-2-yl]-1 -methyl-pyrazole-4-sulfonamide (3.5 mg, 13%). ESI-MS m/z calc. 499.2253, found 500.5 (M+1)⁺; Retention time: 1.92 minutes; LC method A.

### Example 102: Preparation of Compound 319

### Step 1: N-[4-(3-chlorophenoxy)-6-cyclopentyl-pyrimidin-2-yl]-1-methyl-pyrazole-4-sulfonamide (Compound 319)

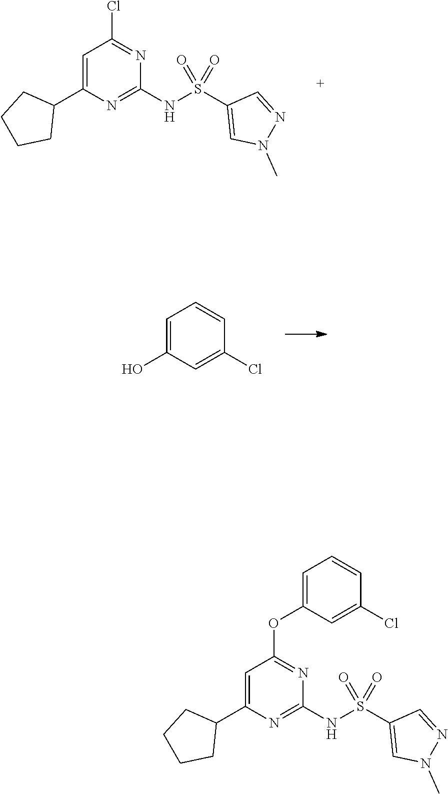 [SMILES: Cn1cc(S(=O)(=O)Nc2nc(Cl)cc(C3CCCC3)n2)cn1 ; Oc1cccc(Cl)c1 ; Cn1cc(S(=O)(=O)Nc2nc(Oc3cccc(Cl)c3)cc(C3CCCC3)n2)cn1]

A solution of N-(4-chloro-6-cyclopentyl-pyrimidin-2-yl)-1-methyl-pyrazole-4-sulfonamide (25 mg, 0.07314 mmol) in NMP (400 μL) was added to 3-chlorophenol (approximately 37.62 mg, 0.2926 mmol). Cesium carbonate (120 mg, 0.3683 mmol) was added, and the reaction mixture was allowed to stir at 100° C. overnight. The reaction mixture was diluted with DMSO and purified by reverse-phase HPLC: Samples were purified using a reverse phase HPLC method using a Luna C₁₈ (2) column (50×21.2 mm, 5 μm particle size) sold by Phenomenex (pn: 00B-4252-PO-AX), and a dual gradient run from 10-70% mobile phase B over 15.0 minutes. Mobile phase A=water (5 mM HCl acid modifier). Mobile phase B=acetonitrile. Flow rate=35 mL/min, injection volume=950 μL, and column temperature=25° C. The UV trace at 220 nm was used to collect fractions to give N-[4-(3-chlorophenoxy)-6-cyclopentyl-pyrimidin-2-yl]-1-methyl-pyrazole-4-sulfonamide (12.1 mg, 38%). ESI-MS m z calc. 433.09753, found 434.2 (M+1)⁺; Retention time: 1.65 minutes; LC method A.

### Example 103: Preparation of Compound 320

### Step 1: Trans-2-amino-6-(2-phenylcyclopropyl)pyrimidin-4-ol

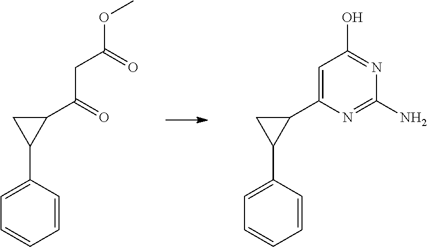 [SMILES: COC(=O)CC(=O)C1CC1c1ccccc1 ; Nc1nc(O)cc(C2CC2c2ccccc2)n1]

Under nitrogen atmosphere, heptane washed sodium (1 g, 43.50 mmol) was dissolved in absolute ethanol (40 mL) and guanidine hydrochloride (3.4 g, 35.59 mmol) was added and the resulting suspension was stirred 5 minutes and then trans-methyl 3-oxo-3-(2-phenylcyclopropyl)propanoate (6 g, 27.49 mmol) was added dissolved in ethanol (3 mL). The reaction was then left stirring at 80° C. for 2 hours then at room temperature overnight. The reaction mixture was concentrated then dissolved in distilled water (250 mL) and the resulting solution was acidified (to pH 3-4) with HCl 3 N and the resulting precipitate, filtered, washed with diethylether(1×50 mL, 2×20 mL). The resulting powder was dried under reduced pressure to provide the pure desired product minutes as a pale yellow powder: trans-2-amino-6-(2-phenylcyclopropyl)pyrimidin-4-ol (5.67 g, 86%) ESI-MS m/z calc. 227.262, found 228.2 (M+1)⁺; Retention time: 1.4. ¹H NMR (300 MHz, DMSO-d₆) ppm 1.20-1.33 (m, 1H), 1.44-1.57 (m, 1H), 1.88-2.02 (m, 1H), 2.28-2.41 (m, 1H), 5.53 (s, 1H), 6.44 (br. s., 2H), 7.06-7.18 (m, 2H), 7.18-7.31 (m, 2H), 10.57 (br. s., 1H). ESI-MS m/z found 228.2 (M+1)+; Retention time: 1.4 minute.

### Step 2: 4-Chloro-6-(2-phenylcyclopropyl)pyrimidin-2-amine

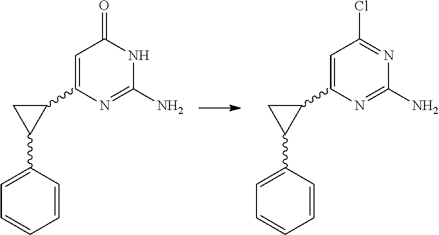 [SMILES: Nc1nc(C2CC2c2ccccc2)cc(=O)[nH]1 ; Nc1nc(Cl)cc(C2CC2c2ccccc2)n1]

A suspension of 2-amino-4-(2-phenylcyclopropyl)-1H-pyrimidin-6-one (5.75 g, 25.3 mmol) in anhydrous dioxane (100 mL) and phosphorus oxychloride (24.0 mL, 25.3 mmol) was added and left stirring 10 minutes at room temperature and then gradually heated up to 80° C. in an oil bath. After heating for 3-3.5 hours the reaction was removed from the oil bath and left to stir at room temperature overnight. The reaction mixture was concentrated under reduced pressure, dissolved in dichloromethane (500 mL), quenched by adding portionwise to 5% aqueous sodium bicarbonate (400 mL) until neutral pH. The aqueous layer was then transferred to a 1.0 L separatory funnel and extracted with dichloromethane (1×300 mL). The combined organic layers were dried over sodium sulfate, filtered on silica gel (3-4 inches) and eluted using 10% ethyl acetate in dichloromethane to provide a purified sample that was further purified via trituration in acetonitrile, filtered and washed with additional acetonitrile then dried under high vacuum to provide the desired product trans-4-chloro-6-(2-phenylcyclopropyl)pyrimidin-2-amine (1.91 g, 30.6%) as a pale yellow solid. A second crop was later isolated by further recrystallization of the concentrated filtrate trans-4-chloro-6-(2-phenylcyclopropyl)pyrimidin-2-amine (610 mg, 9.8%) as pale yellow solid. Total yield: 2.52 g, 40.4%. ¹H NMR (300 MHz, DMSO-d₆) ppm 1.48 (ddd, J=8.3, 6.2, 4.3 Hz, 1H), 1.64 (dt, J=9.3, 4.6 Hz, 1H), 2.12-2.24 (m, 1H), 2.39-2.47 (m, 1H), 6.69 (s, 1H), 6.95 (s, 2H), 7.11-7.20 (m, 3H), 7.21-7.31 (m, 2H). ESI-MS m/z calc. 245.07198, found 246.1 (M+1)⁺; Retention time: 2.99 minutes (LC method H).

### Step 3: 4-(2-Methylphenoxy)-6-(2-phenylcyclopropyl)pyrimidin-2-amine

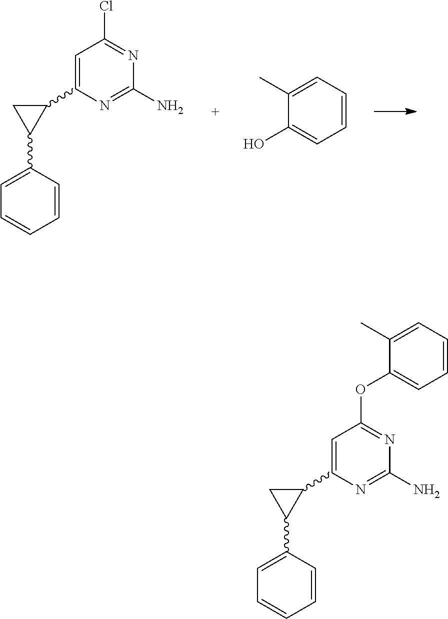

NaH (90 mg of 60% w/w, 2.3 mmol) was added to o-cresol (182.5 mg, 1.688 mmol) in NMP (2 mL) at 0° C. The mixture was stirred for 45 minutes. then added to 4-chloro-6-(2-phenylcyclopropyl)pyrimidin-2-amine (200 mg, 0.814 mmol) in NMP (2 mL). The resulting mixture was stirred at 100° C. for 20 hours. The reaction mixture was cooled down, filtered, and purified on reverse phase HPLC (HCl modifier, 10-60% ACN-H₂O) to give 4-(2-methylphenoxy)-6-(2-phenylcyclopropyl)pyrimidin-2-amine (93.5 mg, 36%). ¹H NMR (400 MHz, DMSO-d₆) δ 7.67-7.40 (m, 1H), 7.38-7.28 (m, 4H), 7.28-7.24 (m, 1H), 7.24-7.16 (m, 4H), 7.12 (d, J=7.8 Hz, 1H), 6.44 (s, 1H), 2.66 (s, 1H), 2.38-2.23 (m, 1H), 2.11 (s, 3H), 1.88 (s, 1H), 1.67 (s, 1H). ESI-MS m/z calc. 317.1528, found 318.0 (M+1)⁺; Retention time: 1.39 minutes; LC method A.

### Step 4: 1-Methyl-N-[4-(2-methylphenoxy)-6-(2-phenylcyclopropyl)pyrimidin-2-yl]pyrazole-4-sulfonamide (Compound 320)

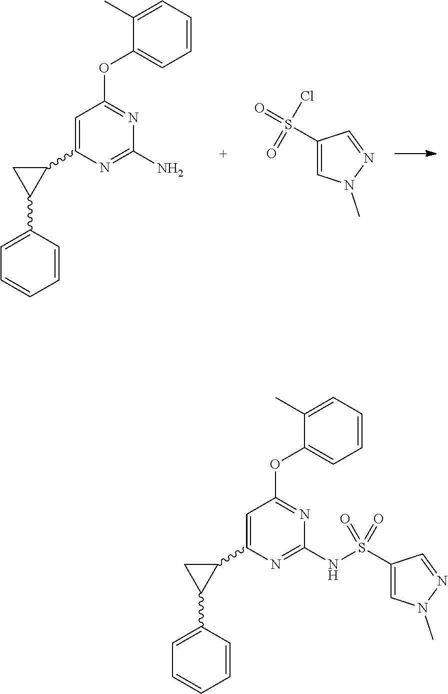

To a solution of 4-(2-methylphenoxy)-6-(2-phenylcyclopropyl)pyrimidin-2-amine (53.5 mg, 0.1669 mmol) in DMF (1 mL) at 0° C. was added NaH (31 mg of 60% w/w, 0.7751 mmol), and the reaction mixture was stirred at this temperature for 5 minutes. The reaction mixture was removed from the cooling bath and stirred at room temperature for 10 minutes. 1-methylpyrazole-4-sulfonyl chloride (42 mg, 0.2325 mmol) in DMF (1 mL) was added slowly to the previous mixture and the resulting mixture was stirred at 60° C. for 10 minutes. LC/MS showed both product and starting material. The crude was filtered and purified by purified on reverse phase HPLC (HCl modifier, 25-75% ACN-H₂O) to give 1-methyl-N-[4-(2-methylphenoxy)-6-(2-phenylcyclopropyl)pyrimidin-2-yl]pyrazole-4-sulfonamide (10.6 mg, 14%)¹H NMR (400 MHz, DMSO-d₆) δ 11.42 (s, 1H), 7.66 (s, 1H), 7.41-7.36 (m, 1H), 7.35-7.23 (m, 5H), 7.22-7.11 (m, 4H), 6.63 (s, 1H), 3.79 (s, 3H), 2.44-2.36 (m, 1H), 2.35-2.28 (m, 1H), 2.10 (s, 3H), 1.71-1.61 (m, 1H), 1.59-1.47 (m, 1H). ESI-MS m/z calc. 461.15216, found 462.0 (M+1)⁺; Retention time: 1.84 minutes; LC method A.

### Example 104: Preparation of Compound 321

### Step 1: 2,2-Dimethyl-5-(1-phenylcyclopropanecarbonyl)-1,3-dioxane-4,6-dione

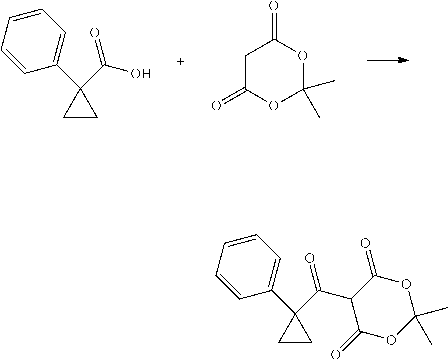

A solution of 1-phenylcyclopropanecarboxylic acid (7.16 g, 44.2 mmol), 2,2-dimethyl-1,3-dioxane-4,6-dione (7.00 g, 48.6 mmol) and 4-(dimethylamino)pyridine (8.09 g, 66.2 mmol) in dichloromethane (75 mL) was cooled in an ice bath and treated with N-(3-dimethylaminopropyl)-N′-ethylcarbodiimide hydrochloride (11.9 g, 62.1 mmol). After 5 minutes, the ice bath was removed, and the reaction was stirred at room temperature over 68 hours. The crude material was transferred to a 1.0-L separatory funnel with water (400 mL) and dichloromethane (about 200 mL). After extraction, the layers were separated and the organic layer was washed with 1 N HCl (2×100 mL), brine (100 mL), dried over sodium sulfate, filtered and concentrated under reduced pressure to afford crude 2,2-dimethyl-5-(1-phenylcyclopropanecarbonyl)-1,3-dioxane-4,6-dione (11.06 g, 85% purity, 74% yield) as an orange solid. ¹H NMR (300 MHz, CDCl₃) ppm 1.46-1.50 (m, 2H), 1.52-1.56 (m, 2H), 1.60 (s, 6H), 7.18-7.31 (m, 3H), 7.37-7.42 (m, 2H). ESI-MS m/z calc. 288.09976, found 287.1 (M−1)⁺; Retention time: 2.17 minutes (LC method C).

### Step 2: Methyl 3-oxo-3-(1-phenylcyclopropyl)propanoate

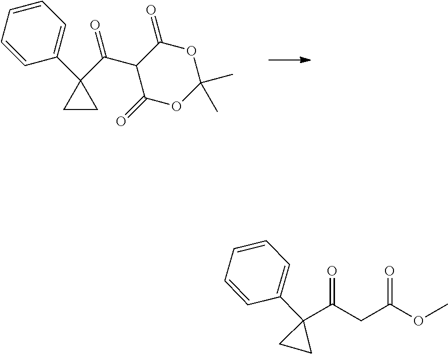 [SMILES: CC1(C)OC(=O)C(C(=O)C2(c3ccccc3)CC2)C(=O)O1 ; COC(=O)CC(=O)C1(c2ccccc2)CC1]

A solution of crude 2,2-dimethyl-5-(1-phenylcyclopropanecarbonyl)-1,3-dioxane-4,6-dione (11.06 g, 38.36 mmol) in methanol (120 mL) was refluxed for 21 hours. The reaction mixture was concentrated under reduced pressure and kept under high vacuum to afford crude methyl 3-oxo-3-(1-phenylcyclopropyl)propanoate (8.51 g, 93% purity, 95% yield) as an orange oil that was used in the following step without further purification. ¹H NMR (300 MHz, CDCl₃) ppm 1.23-1.29 (m, 2H), 1.67-1.73 (m, 2H), 3.35 (s, 2H), 3.64 (s, 3H), 7.26-7.40 (m, 5H). ESI-MS m/z calc. 218.0943, found 219.2 (M+1)⁺; Retention time: 1.95 minutes; LC method C.

### Step 3: 2-Amino-4-(1-phenylcyclopropyl)-1H-pyrimidin-6-one

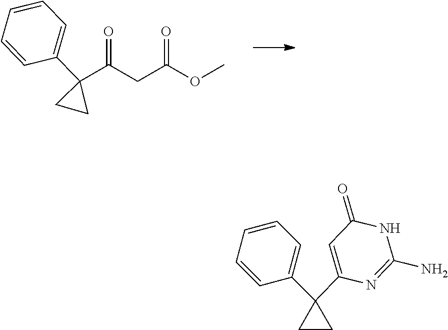 [SMILES: COC(=O)CC(=O)C1(c2ccccc2)CC1 ; Nc1nc(C2(c3ccccc3)CC2)cc(=O)[nH]1]

A flame-dried 500-mL flask was charged with anhydrous ethanol (75 mL) and sodium metal (1.46 g, 63.5 mmol, pre-washed with heptanes) was added. Once gas evolution had stopped and all the sodium had reacted, guanidine hydrochloride (5.78 g, 60.5 mmol) was added (note: a milky white suspension appears). After 5 minutes, a solution of crude methyl 3-oxo-3-(1-phenylcyclopropyl)propanoate (8.51 g, 39.0 mmol) in anhydrous ethanol (5 mL, +5 mL rinse) was added and the reaction was heated in an oil bath at 80° C. for 18 hours. Once cooled, the solvent was removed under reduced pressure, suspended in water (100 mL) and acidified to a pH of 1-2 with concentrated HCl. The solids were filtered, washed with water and dried under high vacuum to afford 2-amino-4-(1-phenylcyclopropyl)-1H-pyrimidin-6-one (6.41 g, 72% yield) as a pale yellow solid. ¹H NMR (300 MHz, DMSO-d₆) ppm 1.04-1.11 (m, 2H), 1.47-1.55 (m, 2H), 4.76 (s, 1H), 6.40 (br.s, 2H), 7.24-7.40 (m, 5H), 10.5 (br.s, 1H). ESI-MS m z calc. 227.10587, found 228.2 (M+1)⁺; Retention time: 1.36 minutes; LC method C.

### Step 4: 4-Chloro-6-(1-phenylcyclopropyl)pyrimidin-2-amine

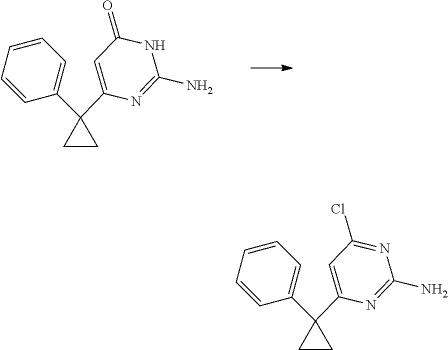 [SMILES: Nc1nc(C2(c3ccccc3)CC2)cc(=O)[nH]1 ; Nc1nc(Cl)cc(C2(c3ccccc3)CC2)n1]

A suspension of 2-amino-4-(1-phenylcyclopropyl)-1H-pyrimidin-6-one (3.50 g, 15.4 mmol) in dioxane (56 mL) and phosphorus oxychloride (14 mL) was gradually heated up to 80° C. in an oil bath (upon heating, turns to an amber solution). After heating for 3-3.5 hours the reaction was removed from the oil bath and left to stir at room temperature overnight (about 17 hours). The reaction was quenched by adding portionwise to 5% aqueous sodium bicarbonate (700 mL) cooled in an ice bath (note: solid sodium hydroxide was added in order to maintain the pH at about 7-8). The aqueous layer was then transferred to a 1.0-L separatory funnel and extracted with dichloromethane (3×150 mL). The combined organic layers were dried over sodium sulfate, filtered and concentrated under reduced pressure. The residue was purified by silica gel chromatography on an 80-g column, eluting from 0% to 40% ethyl acetate in heptanes, to afford 4-chloro-6-(1-phenylcyclopropyl)pyrimidin-2-amine (2.39 g, 98.3% purity, 62% yield) as a pale brown solid. ¹H NMR (300 MHz, DMSO-d₆) ppm 1.21-1.30 (m, 2H), 1.54-1.63 (m, 2H), 5.79 (s, 1H), 7.01 (br. s, 2H), 7.28-7.45 (m, 5H). ESI-MS m/z calc. 245.07198, found 246.1 (M+1)⁺; Retention time: 3.11 minutes; LC method H.

### Step 5: 4-(2-Methylphenoxy)-6-(1-phenylcyclopropyl)pyrimidin-2-amine

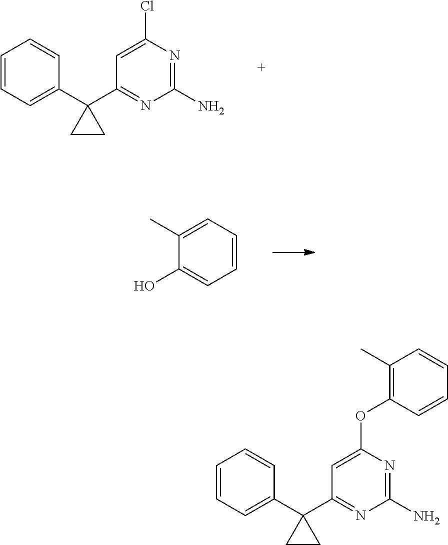

NaH (100 mg of 60% w/w, 2.500 mmol) was added to o-cresol (180 mg, 1.665 mmol) in NMP (3 mL) at 0° C. The mixture was stirred for 45 minutes and then added to 4-chloro-6-(1-phenylcyclopropyl)pyrimidin-2-amine (200 mg, 0.8140 mmol) in NMP (3 mL). The resulting mixture was stirred at 100° C. for 2 hours. The reaction mixture was cooled down, filtered, and purified on reverse phase HPLC (HCl modifier, 15-75% ACN-H₂O) to give 4-(2-methylphenoxy)-6-(1-phenylcyclopropyl)pyrimidin-2-amine (86.2 mg, 33%) ¹H NMR (400 MHz, DMSO-d₆) δ 7.75 (s, 1H), 7.48-7.42 (m, 3H), 7.42-7.36 (m, 2H), 7.36-7.32 (m, 1H), 7.32-7.27 (m, 1H), 7.27-7.21 (m, 1H), 7.21-7.15 (m, 1H), 7.13-7.05 (m, 1H), 5.99 (s, 1H), 2.07 (s, 3H), 1.72 (q, J=4.6, 4.1 Hz, 2H), 1.40 (s, 2H). ESI-MS m/z calc. 317.1528, found 318.0 (M+1)⁺; Retention time: 1.41 minutes; LC method A.

### Step 6: 1-Methyl-N-[4-(2-methylphenoxy)-6-(1-phenylcyclopropyl)pyrimidin-2-yl]pyrazole-4-sulfonamide (Compound 321)

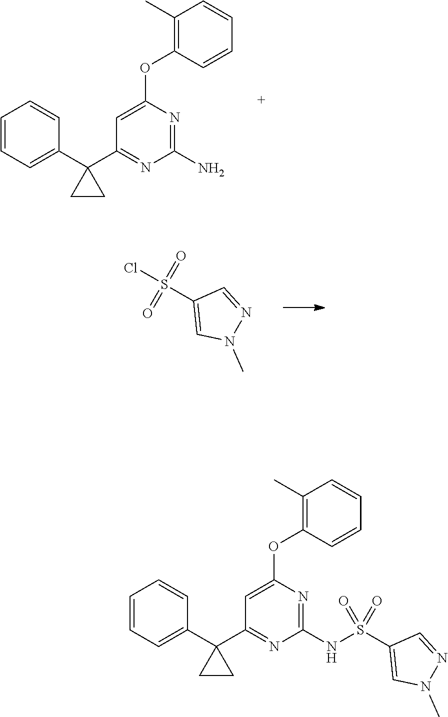

To a solution of 4-(2-methylphenoxy)-6-(1-phenylcyclopropyl)pyrimidin-2-amine (40 mg, 0.1260 mmol) in DMF (500 μL) at 0° C. was added NaH (20 mg of 60% w/w, 0.5000 mmol) and the reaction mixture was stirred at this temperature for 5 minutes. The reaction mixture was removed from the cooling bath and stirred at room temperature for 30 minutes. 1-methylpyrazole-4-sulfonyl chloride (45 mg, 0.2492 mmol) in DMF (500 μL) was added slowly to the previous mixture and the resulting mixture was stirred at 65° C. for 1 hour. LC/MS showed trace of product and starting material. The reaction mixture was stirred overnight at room temperature. Then the temperature was increased to 60° C. and the reaction mixture was stirred at this temperature for 20 minutes. The crude was filtered and purified by reverse phase HPLC (HCl modifier, 15-75% ACN-H₂O) to give 1-methyl-N-[4-(2-methylphenoxy)-6-(1-phenylcyclopropyl)pyrimidin-2-yl]pyrazole-4-sulfonamide (3 mg, 5%) ESI-MS m/z calc. 461.15216, found 461.0 (M+1)⁺; Retention time: 1.92 minutes; LC method A.

### Example 105: Characterization of Compounds 322-342

The compounds in the following tables were prepared in a manner analogous to that described above using commercially available reagents and intermediates described herein.

| | | LCMS | | | | |
|---|---|---|---|---|---|---|
| | | Room | | | | |
| Cmpd | | Temperature | Calc. | | LCMS | |
| No. | Structure | (min) | mass | M + 1 | Met. | NMR |
| 322 | 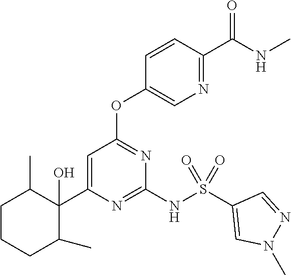 [SMILES: CNC(=O)c1ccc(Oc2cc(C3(O)C(C)CCCC3C)nc(NS(=O)(=O)c3cnn(C)c3)n2)cn1] | 1.39 | 515.195 | 516.47 | A | |
| 323 | 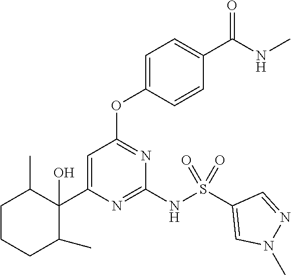 [SMILES: CNC(=O)c1ccc(Oc2cc(C3(O)C(C)CCCC3C)nc(NS(=O)(=O)c3cnn(C)c3)n2)cc1] | 1.38 | 514.2 | 515.49 | A | |
| 324 | 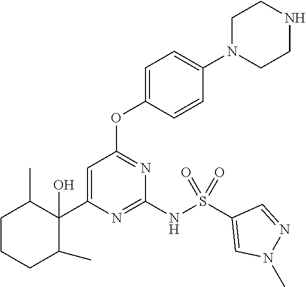 [SMILES: CC1CCCC(C)C1(O)c1cc(Oc2ccc(N3CCNCC3)cc2)nc(NS(=O)(=O)c2cnn(C)c2)n1] | 1.13 | 541.247 | 542.52 | A | |
| 325 | 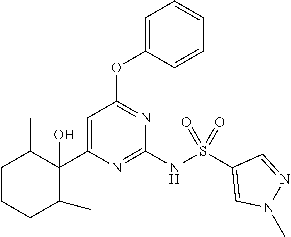 [SMILES: CC1CCCC(C)C1(O)c1cc(Oc2ccccc2)nc(NS(=O)(=O)c2cnn(C)c2)n1] | 1.65 | 457.178 | 458.45 | A | |
| 326 | 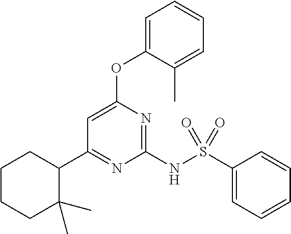 [SMILES: Cc1ccccc1Oc1cc(C2CCCCC2(C)C)nc(NS(=O)(=O)c2ccccc2)n1] | 2.11 | 451.193 | 452.43 | A | |
| 327 | 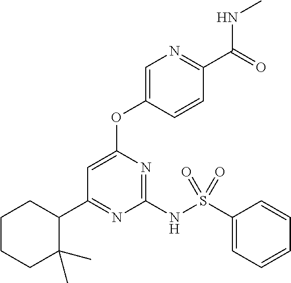 [SMILES: CNC(=O)c1ccc(Oc2cc(C3CCCCC3(C)C)nc(NS(=O)(=O)c3ccccc3)n2)cn1] | 1.73 | 495.194 | 496.47 | A | |
| 328 | 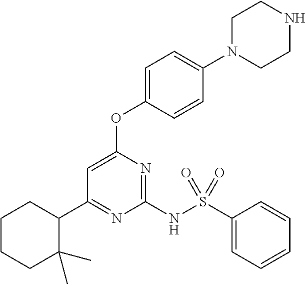 [SMILES: CC1(C)CCCCC1c1cc(Oc2ccc(N3CCNCC3)cc2)nc(NS(=O)(=O)c2ccccc2)n1] | 1.25 | 521.246 | 522.56 | A | |
| 329 | 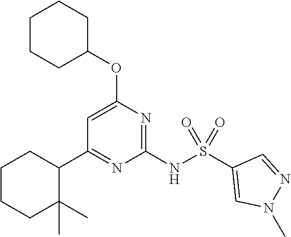 [SMILES: Cn1cc(S(=O)(=O)Nc2nc(OC3CCCCC3)cc(C3CCCCC3(C)C)n2)cn1] | 1.84 | 447.23 | 448.47 | A | |
| 330 | 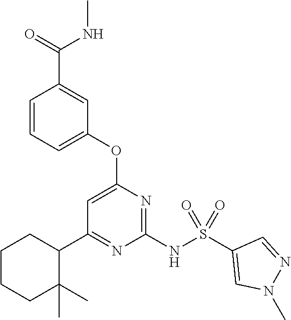 [SMILES: CNC(=O)c1cccc(Oc2cc(C3CCCCC3(C)C)nc(NS(=O)(=O)c3cnn(C)c3)n2)c1] | 1.1 | 498.205 | 499.3 | R | |
| 331 | 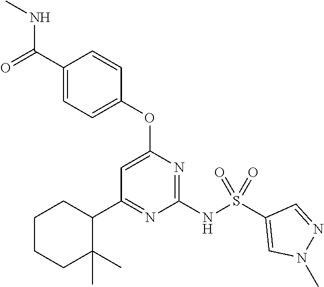 [SMILES: CNC(=O)c1ccc(Oc2cc(C3CCCCC3(C)C)nc(NS(=O)(=O)c3cnn(C)c3)n2)cc1] | 1.08 | 498.205 | 499.33 | R | |
| 332 | 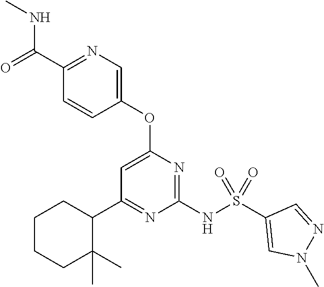 [SMILES: CNC(=O)c1ccc(Oc2cc(C3CCCCC3(C)C)nc(NS(=O)(=O)c3cnn(C)c3)n2)cn1] | 1.1 | 499.2 | 500.32 | R | |
| 333 | 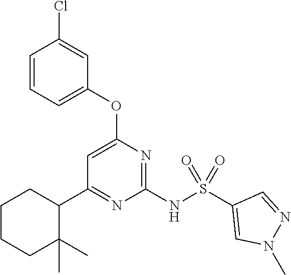 [SMILES: Cn1cc(S(=O)(=O)Nc2nc(Oc3cccc(Cl)c3)cc(C3CCCCC3(C)C)n2)cn1] | 1.4 | 475.144 | 476.27 | R | |
| 334 | 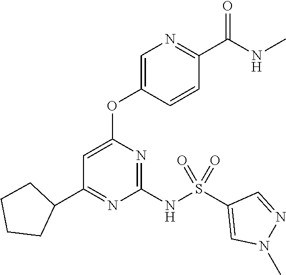 [SMILES: CNC(=O)c1ccc(Oc2cc(C3CCCC3)nc(NS(=O)(=O)c3cnn(C)c3)n2)cn1] | 1.25 | 457.153 | 457.76 | A | |
| 335 | 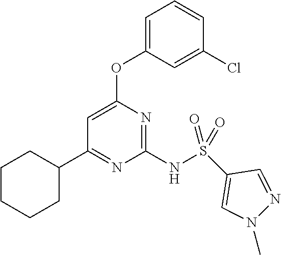 [SMILES: Cn1cc(S(=O)(=O)Nc2nc(Oc3cccc(Cl)c3)cc(C3CCCCC3)n2)cn1] | 1.74 | 447.113 | 448.2 | A | |
| 336 | 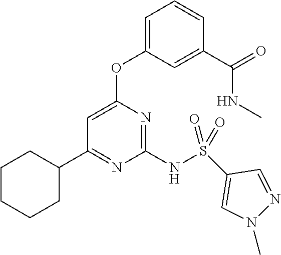 [SMILES: CNC(=O)c1cccc(Oc2cc(C3CCCCC3)nc(NS(=O)(=O)c3cnn(C)c3)n2)c1] | 1.3 | 470.174 | 471.3 | A | |
| 337 | 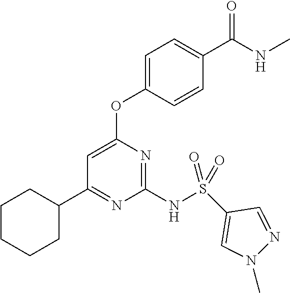 [SMILES: CNC(=O)c1ccc(Oc2cc(C3CCCCC3)nc(NS(=O)(=O)c3cnn(C)c3)n2)cc1] | 1.28 | 470.174 | 471.3 | A | |
| 338 | 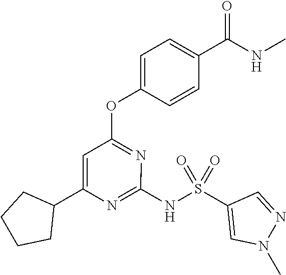 [SMILES: CNC(=O)c1ccc(Oc2cc(C3CCCC3)nc(NS(=O)(=O)c3cnn(C)c3)n2)cc1] | 1.19 | 456.158 | 457.2 | A | ¹H NMR (400 MHz, DMSO- d₆) δ 8.55 (q, J = 4.4 Hz, 1H), 8.04 − 7.93 (m, 2H), 7.61 (s, 1H), 7.41 − 7.32 (m, 2H), 7.29 (s, 1H), 6.56 (s, 1H), 3.73 (s, 3H), 3.01 (p, J = 8.0 Hz, 1H), 2.80 (d, J = 4.5 Hz, 3H), 2.01 − 1.87 (m, |
| | | | | | | 2H), 1.74 |
| | | | | | | (ddq, J = 8.9, |
| | | | | | | 6.9, 3.7, 2.7 |
| | | | | | | Hz, 2H), 1.71 − |
| | | | | | | 1.53 (m, 4H). |
| 339 | 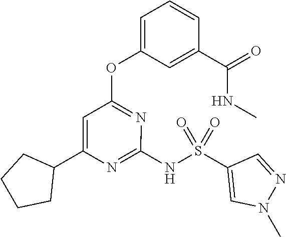 [SMILES: CNC(=O)c1cccc(Oc2cc(C3CCCC3)nc(NS(=O)(=O)c3cnn(C)c3)n2)c1] | 1.21 | 456.158 | 457.1 | A | |
| 340 | 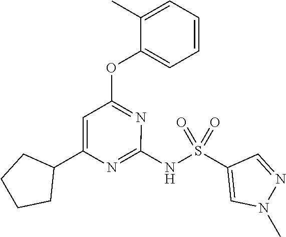 [SMILES: Cc1ccccc1Oc1cc(C2CCCC2)nc(NS(=O)(=O)c2cnn(C)c2)n1] | 1.56 | 413.152 | 414.1 | A | |
| 341 | 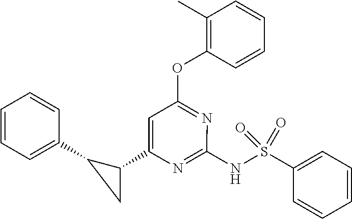 [SMILES: Cc1ccccc1Oc1cc([C@@H]2C[C@@H]2c2ccccc2)nc(NS(=O)(=O)c2ccccc2)n1] | 2.08 | 457.146 | 458 | A | ¹H NMR (400 MHz, DMSO- d₆) 11.61 (s, 1H), 7.66 − 7.53 (m, 3H), 7.47 (s, 2H), 7.39 (d, J = 6.9 Hz, 1H), 7.35 − 7.24 (m, 4H), 7.13 (dt, J = 38.7, 7.6 Hz, 4H), 6.61 (s, 1H), 2.27 (s, 2H), |
| | | | | | | 2.05 (s, 3H), |
| | | | | | | 1.53 (d, J = |
| | | | | | | 32.3 Hz, 2H). |
| 342 | 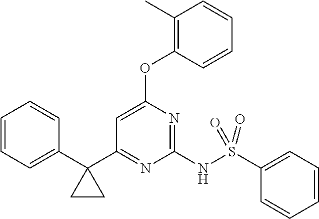 [SMILES: Cc1ccccc1Oc1cc(C2(c3ccccc3)CC2)nc(NS(=O)(=O)c2ccccc2)n1] | 2.16 | 457.146 | 458 | A | ¹H NMR (400 MHz, DMSO- d₆) δ 11.60 (s, 1H), 7.66 − 7.54 (m, 3H), 7.48 (t, J = 7.5 Hz, 2H), 7.35 (td, J = 13.4, 12.5, 6.7 Hz, 6H), 7.29 − 7.18 (m, 2H), 7.00 (d, J = 6.9 Hz, 1H), 5.73 (s, |
| | | | | | | 1H), 1.98 (s, |
| | | | | | | 3H), 1.41 (s, |
| | | | | | | 2H), 1.24 (s, |
| | | | | | | 2H). |

### Example 106: Preparation of Compound 343

### Step 1: 5-[6-Chloro-2-[(1-methylpyrazol-4-yl)sulfonylamino]pyrimidin-4-yl]oxy-N-methyl-pyridine-2-carboxamide

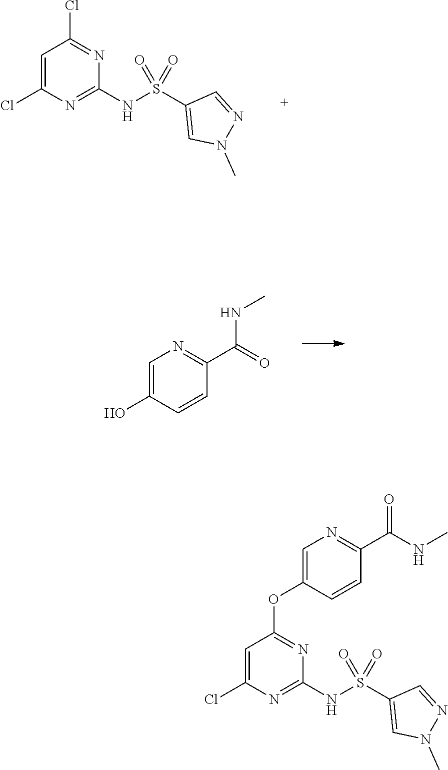 [SMILES: Cn1cc(S(=O)(=O)Nc2nc(Cl)cc(Cl)n2)cn1 ; CNC(=O)c1ccc(O)cn1 ; CNC(=O)c1ccc(Oc2cc(Cl)nc(NS(=O)(=O)c3cnn(C)c3)n2)cn1]

To a solution of N-(4,6-dichloropyrimidin-2-yl)-1-methyl-pyrazole-4-sulfonamide (approximately 1.311 g, 4.256 mmol) and 5-hydroxy-N-methyl-pyridine-2-carboxamide (518.0 mg, 3.405 mmol) in NMP (5.506 mL) was added potassium carbonate (approximately 2.352 g, 17.02 mmol). The solution was heated to 110° C. for 16 hours. The reaction mixture was diluted with water and 5 mL of acetic acid was added. The aqueous layer was extracted with ethyl acetate/hexanes (1:1, 5×). The combined organics were washed with brine, dried with magnesium sulfate, filtered, and concentrated in vacuo. The crude residue was purified by flash column chromatography on silica gel (100% ethyl acetate). 5-[6-Chloro-2-[(1-methylpyrazol-4-yl)sulfonylamino]pyrimidin-4-yl]oxy-N-methyl-pyridine-2-carboxamide (1.03 g, 50%) was isolated as a mixture of three compounds (2.8:1:0.19, UV-Vis integration). ESI-MS m/z calc. 423.05164, found 424.31 (M+1)⁺; Retention time: 0.47 minutes; LC method D.

### Step 2: N-methyl-5-[6-(6-methylcyclohexen-1-yl)-2-[(1-methylpyrazol-4-yl)sulfonylamino]pyrimidin-4-yl]oxy-pyridine-2-carboxamide (Compound 344)

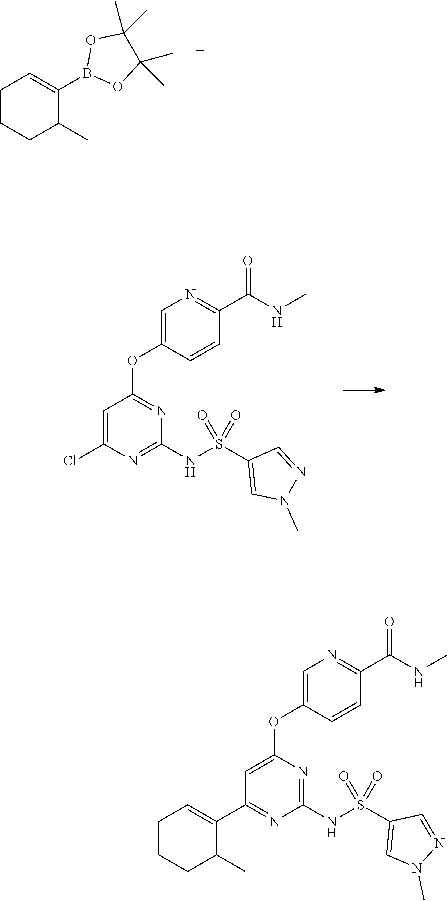 [SMILES: CC1CCCC=C1B1OC(C)(C)C(C)(C)O1 ; CNC(=O)c1ccc(Oc2cc(Cl)nc(NS(=O)(=O)c3cnn(C)c3)n2)cn1 ; CNC(=O)c1ccc(Oc2cc(C3=CCCCC3C)nc(NS(=O)(=O)c3cnn(C)c3)n2)cn1]

A heterogeneous mixture of 4,4,5,5-tetramethyl-2-(6-methylcyclohexen-1-yl)-1,3,2-dioxaborolane (approximately 39.98 mg, 0.1800 mmol), 5-[6-chloro-2-[(1-methylpyrazol-4-yl)sulfonylamino]pyrimidin-4-yl]oxy-N-methyl-pyridine-2-carboxamide (42.38 mg, 0.10 mmol), cesium carbonate (approximately 162.9 mg, 0.5000 mmol), and bis(triphenylphosphine)palladium(II) dichloride(approximately 14.04 mg, 0.02000 mmol) in 1.00 mL dioxane/water (10:1, 0.1M) was heated to 100° C. for 12 hours. The organic layers were separated from the aqueous layer and the solvent was evaporated under a stream of air. To the crude residues were added 0.020 mL of acetic acid and 0.50 mL of dimethyl sulfoxide. The resulting mixture was filtered and separated by reverse phase chromatography (gradient: 1-99% CH₃CN in H₂O with 0.1% hydrochloric acid). N-methyl-5-[6-(6-methylcyclohexen-1-yl)-2-[(1-methylpyrazol-4-yl)sulfonylamino]pyrimidin-4-yl]oxy-pyridine-2-carboxamide (12.5 mg, 26%). ESI-MS m/z calc. 483.16888, found 484.46 (M+1)⁺; Retention time: 1.55 minutes; LC method A.

### Step 3: N-methyl-5-[6-(2-methylcyclohexyl)-2-[(1-methylpyrazol-4-yl)sulfonylamino]pyrimidin-4-yl]oxy-pyridine-2-carboxamide (Compound 343)

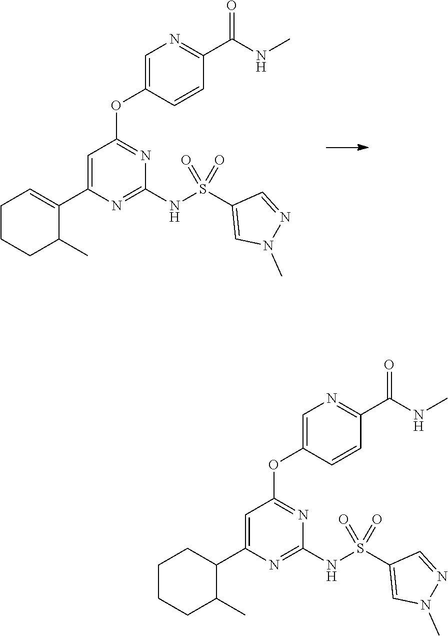 [SMILES: CNC(=O)c1ccc(Oc2cc(C3=CCCCC3C)nc(NS(=O)(=O)c3cnn(C)c3)n2)cn1 ; CNC(=O)c1ccc(Oc2cc(C3CCCCC3C)nc(NS(=O)(=O)c3cnn(C)c3)n2)cn1]

To a solution of N-methyl-5-[6-(6-methylcyclohexen-1-yl)-2-[(1-methylpyrazol-4-yl)sulfonylamino]pyrimidin-4-yl]oxy-pyridine-2-carboxamide (10 mg, 0.02068 mmol) in 1.0 mL of methanol and 2.0 mL of ethyl acetate was added 10% palladium on carbon (approximately 22.01 mg of 10% w/w, 0.02068 mmol). The solution was stirred under an atmosphere of hydrogen gas at 23° C. for 14 hours. The solvent was removed in vacuo and the crude residue was dissolved in 1.0 mL of dimethyl sulfoxide and filtered. The separation was carried out by reverse phase chromatography (eluent: acetonitrile/water with 0.1% hydrochloric acid). N-methyl-5-[6-(2-methylcyclohexyl)-2-[(1-methylpyrazol-4-yl)sulfonylamino]pyrimidin-4-yl]oxy-pyridine-2-carboxamide (4.9 mg, 49%). ESI-MS m/z calc. 485.1845, found 486.19 (M+1)⁺; Retention time: 1.48 minutes; LC method A.

### Example 107: Preparation of Compound 345

### Step 1: 5-[6-(6,6-Dimethyl-2,5-dihydropyran-4-yl)-2-[(1-methylpyrazol-4-yl)sulfonylamino]pyrimidin-4-yl]oxy-N-methyl-pyridine-2-carboxamide

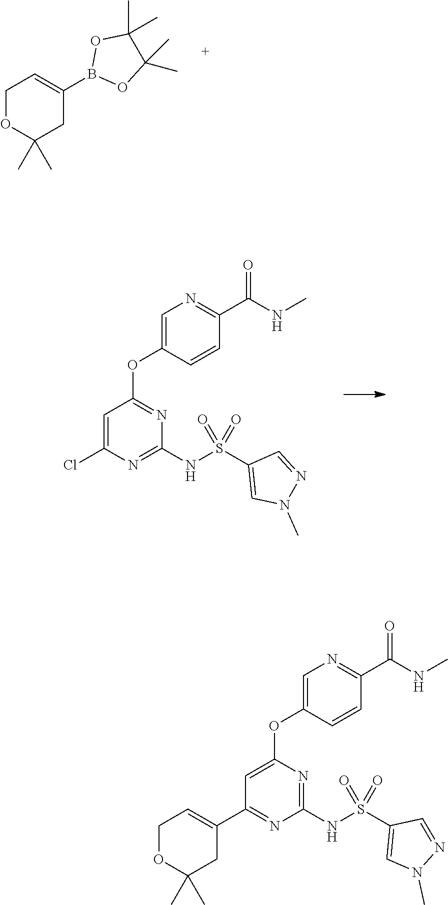 [SMILES: CC1(C)CC(B2OC(C)(C)C(C)(C)O2)=CCO1 ; CNC(=O)c1ccc(Oc2cc(Cl)nc(NS(=O)(=O)c3cnn(C)c3)n2)cn1 ; CNC(=O)c1ccc(Oc2cc(C3=CCOC(C)(C)C3)nc(NS(=O)(=O)c3cnn(C)c3)n2)cn1]

A heterogeneous mixture of 2-(2,2-dimethyl-3,6-dihydro-2H-pyran-4-yl)-4,4,5,5-tetramethyl-1,3,2-dioxaborolane (approximately 42.86 mg, 0.1800 mmol), 5-[6-chloro-2-[(1-methylpyrazol-4-yl)sulfonylamino]pyrimidin-4-yl]oxy-N-methyl-pyridine-2-carboxamide (42.38 mg, 0.10 mmol), cesium carbonate (approximately 162.9 mg, 0.5000 mmol), and bis(triphenylphosphine)palladium(II) dichloride(approximately 14.04 mg, 0.02000 mmol) in 1.00 mL dioxane/water (10:1, 0.1M) was heated to 100° C. for 12 hours. The organic layers were separated from the aqueous layer and the solvent concentrated under a stream of air. To the crude residues were added 0.020 mL of acetic acid and 0.50 mL of dimethyl sulfoxide. The resulting mixture was filtered and separated by reverse phase chromatography (gradient: 1-99% CH₃CN in H₂O with 0.1% hydrochloric acid). 5-[6-(6,6-Dimethyl-2,5-dihydropyran-4-yl)-2-[(1-methylpyrazol-4-yl)sulfonylamino]pyrimidin-4-yl]oxy-N-methyl-pyridine-2-carboxamide (10 mg, 20%). ESI-MS m/z calc. 499.1638, found 500.5 (M+1)⁺; Retention time: 1.21 minutes; LC method A.

### Step 2: 5-[6-(2,2-Dimethyltetrahydropyran-4-yl)-2-[(1-methylpyrazol-4-yl)sulfonylamino]pyrimidin-4-yl]oxy-N-methyl-pyridine-2-carboxamide (Compound 345)

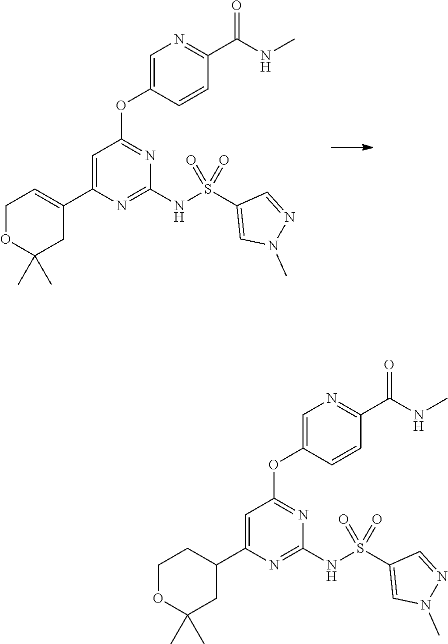 [SMILES: CNC(=O)c1ccc(Oc2cc(C3=CCOC(C)(C)C3)nc(NS(=O)(=O)c3cnn(C)c3)n2)cn1 ; CNC(=O)c1ccc(Oc2cc(C3CCOC(C)(C)C3)nc(NS(=O)(=O)c3cnn(C)c3)n2)cn1]

To a solution of 5-[6-(6,6-dimethyl-2,5-dihydropyran-4-yl)-2-[(1-methylpyrazol-4-yl)sulfonylamino]pyrimidin-4-yl]oxy-N-methyl-pyridine-2-carboxamide (10 mg, 0.02002 mmol) in 1.0 mL of methanol and 2.0 mL of ethyl acetate was added 10% palladium on carbon (approximately 21.31 mg of 10% w/w, 0.02002 mmol). The solution was stirred under an atmosphere of hydrogen gas at 23° C. for 14 hours. The solvent was removed in vacuo and the crude residue was dissolved in 1.0 mL of dimethyl sulfoxide and filtered. The separation was carried out by reverse phase chromatography (eluent: acetonitrile/water with 0.1% hydrochloric acid). 5-[6-(2,2-Dimethyltetrahydropyran-4-yl)-2-[(1-methylpyrazol-4-yl)sulfonylamino]pyrimidin-4-yl]oxy-N-methyl-pyridine-2-carboxamide (2.9 mg, 29%). ESI-MS m/z calc. 501.17944, found 502.36 (M+1)⁺; Retention time: 1.13 minutes; LC method A.

### Example 108: Preparation of Compound 346

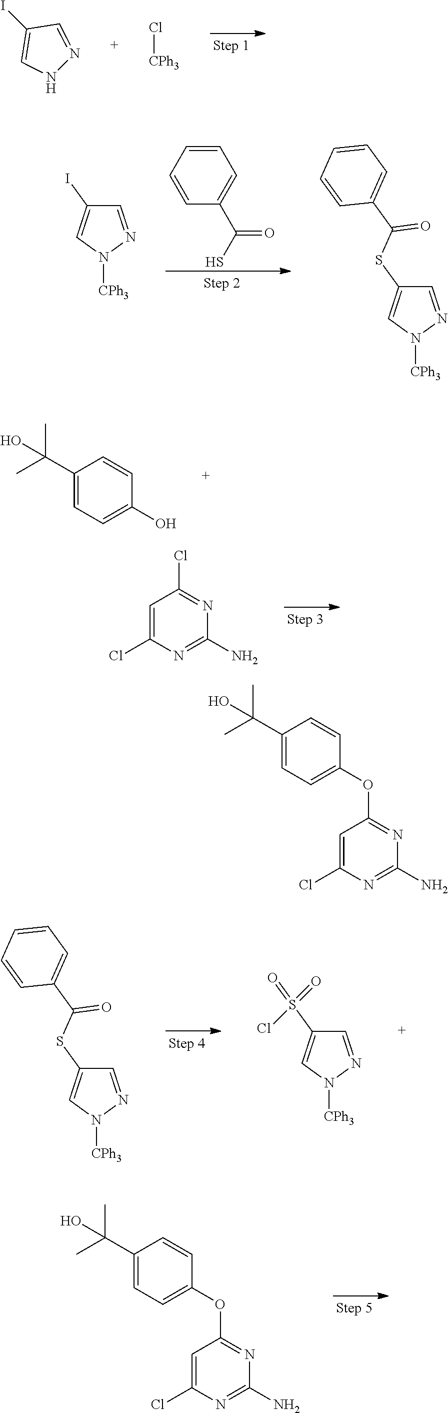

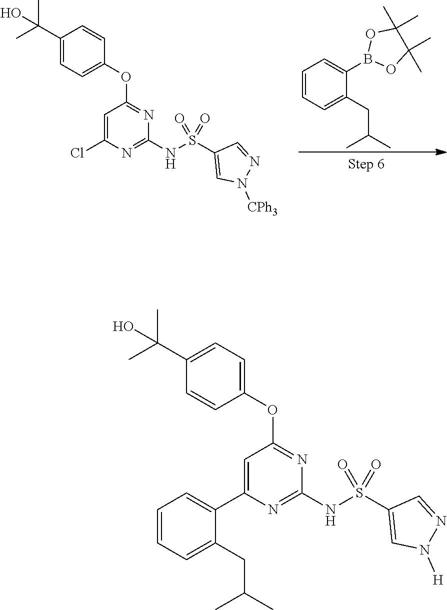

### Step 1: 4-Iodo-1-trityl-pyrazole

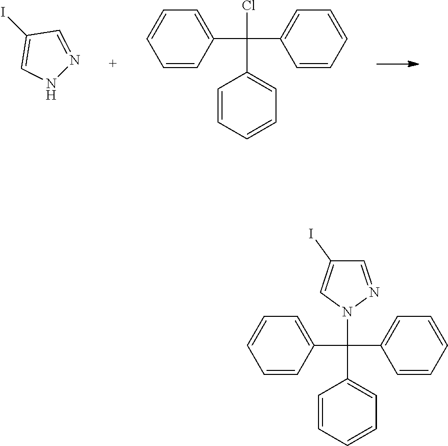 [SMILES: Ic1cn[nH]c1 ; ClC(c1ccccc1)(c1ccccc1)c1ccccc1 ; Ic1cnn(C(c2ccccc2)(c2ccccc2)c2ccccc2)c1]

Chloro(diphenyl)methyl]benzene (47.5 g, 170.4 mmol) was added to a solution of 4-iodo-1H-pyrazole (30.0 g, 154.7 mmol) and triethylamine (43.2 mL, 309.9 mmol) in dichloromethane (300 mL) at 0° C. The solution was stirred at room temperature for 24 hours. Water (500 mL) was added and the aqueous phase was extracted with dichloromethane (3×300 mL). The organic layers were combined, washed with sodium bicarbonate solution (saturated, 300 mL) and brine (300 mL), dried with anhydrous sodium sulfate, filtered and concentrated under reduced pressure. The crude compound (74.76 g of off-white solid) was recrystallized in a mix of heptanes (800 mL) and ethyl acetate (780 mL) to afford 4-iodo-1-trityl-pyrazole (56.64 g, 84%) as off-white solid. ¹H NMR (300 MHz, CDCl₃) 7.67 (s, 1H), 7.41 (s, 1H), 7.36-7.27 (m, 9H), 7.16-7.09 (m, 6H).

### Step 2: S-(1-tritylpyrazol-4-yl) benzenecarbothioate

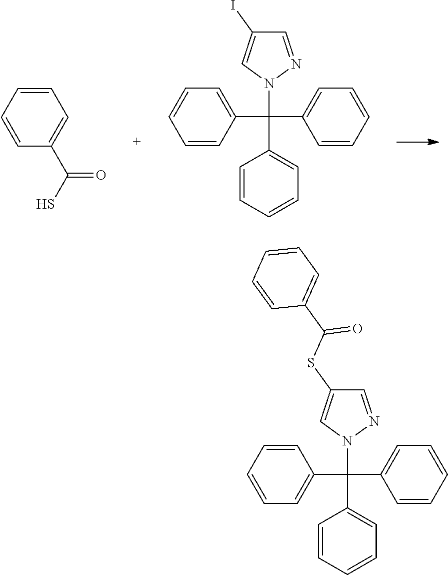

To a solution of 4-iodo-1-trityl-pyrazole (10.0 g, 22.9 mmol), copper iodide (437 mg, 22.9 mmol), and 1,10-phenanthroline (826 mg, 4.58 mmol) in toluene (50.0 mL) was added benzenecarbothioic S-acid (3.24 mL, 27.5 mmol), followed by diisopropylethylamine (7.98 mL, 45.8 mmol) and the mixture was heated at 110° C. overnight. After completion, the mixture was cooled to room temperature and quenched by adding water (100.0 mL). The mixture was extracted with ethyl acetate (2×70.0 mL) and the combined organic layers were washed with brine (80.0 mL), dried over sodium sulfate, filtered, and concentrated under reduced pressure. The obtained residue was filtered through a plug of silica with dichloromethane as an eluent and concentrated under reduced pressure. The residue was then taken up with 10% ethyl acetate in heptane and filtered through Buchner funnel. The solid was then washed with heptane to give S-(1-tritylpyrazol-4-yl) benzenecarbothioate (8.2 g, 80.1%) as a white solid. ¹H NMR (300 MHz, DMSO-d₆) 7.97-7.90 (m, 2H), 7.80 (s, 1H), 7.75-7.67 (m, 1H), 7.61-7.53 (m, 3H), 7.42-7.32 (m, 9H), 7.07 (dd, J=7.5, 2.2 Hz, 6H).

### Step 3: 2-[4-(2-Amino-6-chloro-pyrimidin-4-yl)oxyphenyl]propan-2-ol

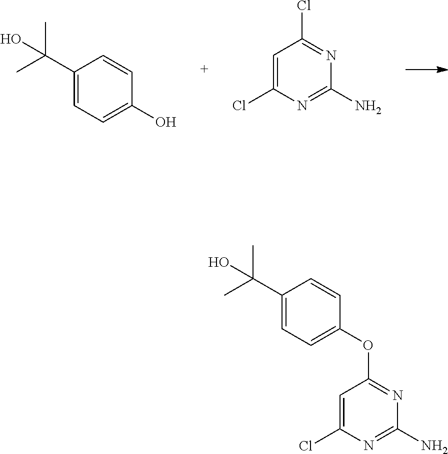 [SMILES: CC(C)(O)c1ccc(O)cc1 ; Nc1nc(Cl)cc(Cl)n1 ; CC(C)(O)c1ccc(Oc2cc(Cl)nc(N)n2)cc1]

4,6-Dichloropyrimidin-2-amine (1 g, 6 mmol), 4-(1-hydroxy-1-methyl-ethyl)phenol (975 mg, 6.41 mmol), DMF (10 mL), and K₂CO₃ (2.54 g, 18.4 mmol) were combined and stirred at 100° C. for 3 hours. The reaction mixture was cooled down to room temperature, and the pH of the mixture was adjusted to ˜5 with 1 N HCl, extracted with ethyl acetate (3×10 ml). The combined organic layers were extracted with brine, dried over Na₂SO₄, concentrated and purified on silica using a gradient of ethyl acetate and hexane. The product came out ˜ 30% ethyl acetate to give 2-[4-(2-amino-6-chloro-pyrimidin-4-yl)oxyphenyl]propan-2-ol (1.52 g, 89%) as a white solid. ¹H NMR (400 MHz, DMSO-d₆) δ 7.50 (d, J=5.3 Hz, 2H), 7.14 (s, 2H), 7.09 (d, J=8.6 Hz, 2H), 6.19 (s, 1H), 5.05 (s, 1H), 1.43 (s, 6H). ESI-MS m/z calc. 279.07745, found 280.0 (M+1)⁺; Retention time: 1.2 minutes; LC method A.

### Step 4: 1-Tritylpyrazole-4-sulfonyl chloride

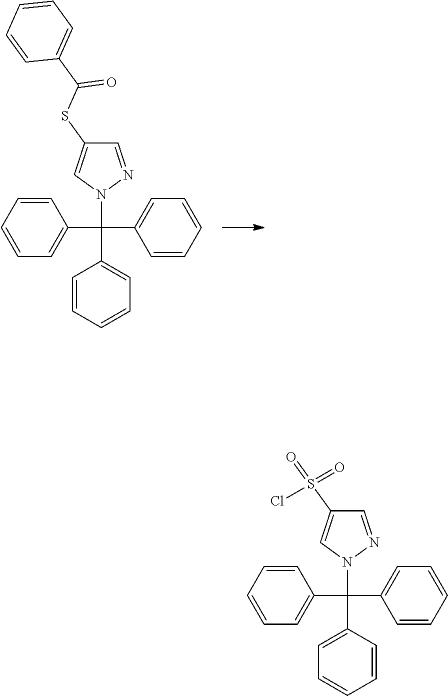 [SMILES: O=C(Sc1cnn(C(c2ccccc2)(c2ccccc2)c2ccccc2)c1)c1ccccc1 ; O=S(=O)(Cl)c1cnn(C(c2ccccc2)(c2ccccc2)c2ccccc2)c1]

To a white suspension of benzyltrimethylammonium chloride (10.56 g, 56.87 mmol) in acetonitrile (150 mL) was added trichloroisocyanuric acid (4.53 g, 19.49 mmol) and the mixture was stirred for 0.5 hours. Then, this mixture was added slowly over 10 minutes to a suspension of S-(1-tritylpyrazol-4-yl) benzenecarbothioate (7.24 g, 16.21 mmol) in acetonitrile (150 mL) at 0° C., followed by 1 M aqueous solution of sodium carbonate (33 mL of 1 M, 33.00 mmol). The mixture was stirred at this temperature for 10 minutes, then 45 minutes at room temperature. After completion, the mixture was concentrated under reduced pressure, redissolved in water (300 mL) and extracted with ethyl acetate (3×150 mL). The combined organic layers were washed with brine (80 mL), dried over sodium sulfate, filtered and concentrated under reduced pressure. The residue was purified by flash chromatography on silica gel eluting from 0% to 25% ethyl acetate in heptanes, the product fractions were combined and concentrated under reduced pressure to give white solid, which was then triturated in heptanes. The solid was dissolved into dichloromethane and concentrated under reduced pressure (repeated 2 times) to give 1-tritylpyrazole-4-sulfonyl chloride (2.48 g, 35%) as white solid. ¹H NMR (300 MHz, CDCl₃) 8.12 (s, 1H), 8.04 (d, J=0.9 Hz, 1H), 7.45-7.29 (m, 9H), 7.18-7.05 (m, 6H). ESI-MS m/z calc. 408.06992, no ionization detected, Retention time: 3.93 minutes; LC method H.

### Step 5: N-[4-Chloro-6-[4-(1-hydroxy-1-methyl-ethyl)phenoxy]pyrimidin-2-yl]-1-trityl-pyrazole-4-sulfonamide

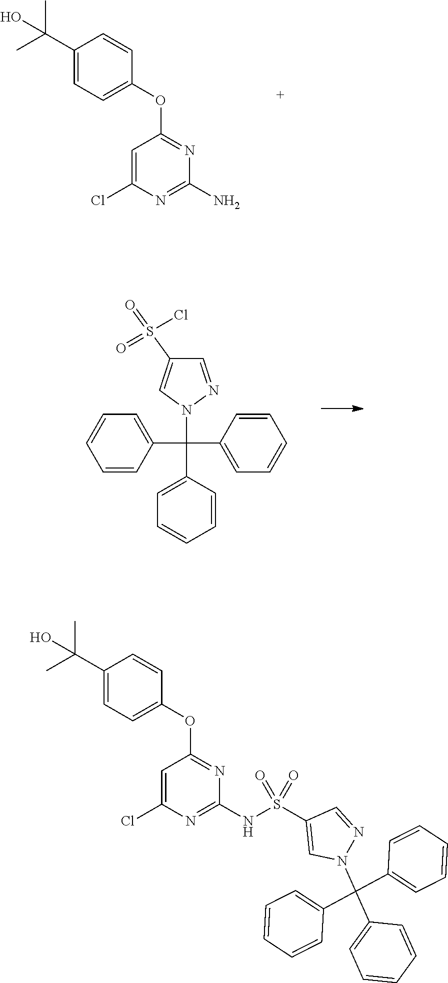 [SMILES: CC(C)(O)c1ccc(Oc2cc(Cl)nc(N)n2)cc1 ; O=S(=O)(Cl)c1cnn(C(c2ccccc2)(c2ccccc2)c2ccccc2)c1 ; CC(C)(O)c1ccc(Oc2cc(Cl)nc(NS(=O)(=O)c3cnn(C(c4ccccc4)(c4ccccc4)c4ccccc4)c3)n2)cc1]

To a solution of 2-[4-(2-amino-6-chloro-pyrimidin-4-yl)oxyphenyl]propan-2-ol (700 mg, 2.477 mmol) in DMF (5 mL) at 0° C. was added NaH (322 mg of 60% w/w, 8.05 mmol) and the reaction mixture was stirred at this temperature for 5 minutes. The reaction mixture was removed from the cooling bath and stirred at room temperature for 30 minutes. Then 1-tritylpyrazole-4-sulfonyl chloride (1 g, 2 mmol) in DMF (5 mL) was added slowly to the previous mixture and the resulting mixture was stirred at room temperature for 2 hours. The reaction mixture was diluted with water and extracted with ethyl acetate (2×10 ml). The combined organic layers were washed with brine, dried over Na₂SO₄, concentrated under reduced pressure. The crude was purified on silica using a gradient of ethyl acetate/hexane. The product came out at ˜30% ethyl acetate: N-[4-chloro-6-[4-(1-hydroxy-1-methyl-ethyl)phenoxy]pyrimidin-2-yl]-1-trityl-pyrazole-4-sulfonamide (1.01 g, 40%). ¹H NMR (400 MHz, Chloroform-d) δ 8.01 (s, 1H), 7.51 (s, 1H), 7.50 (s, 1H), 7.34-7.29 (m, 10H), 7.09-7.01 (m, 10H), 6.54 (s, 1H), 1.48 (s, 6H). ESI-MS m/z calc. 651.1707, found 652.0 (M+1)⁺; Retention time: 2.13 minutes, LC method A.

### Step 6: N-[4-[4-(1-Hydroxy-1-methyl-ethyl)phenoxy]-6-(2-isobutylphenyl)pyrimidin-2-yl]-1H-pyrazole-4-sulfonamide (Compound 346)

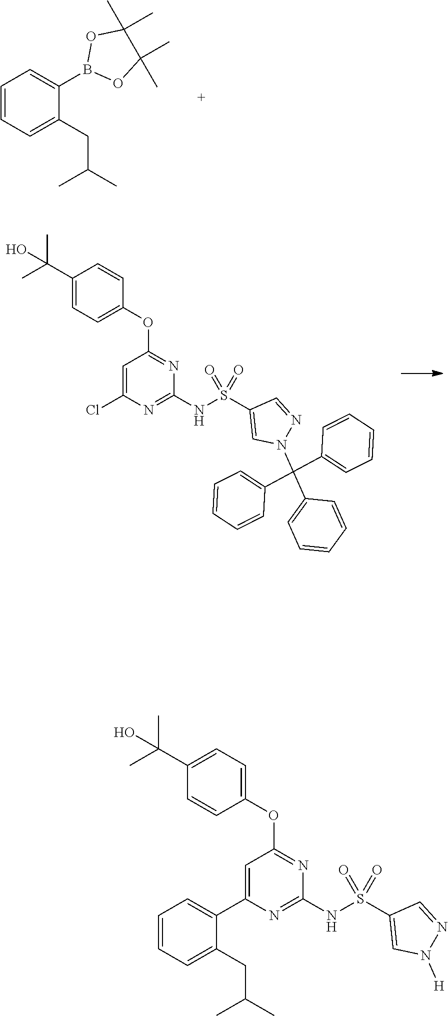 [SMILES: CC(C)Cc1ccccc1B1OC(C)(C)C(C)(C)O1 ; CC(C)(O)c1ccc(Oc2cc(Cl)nc(NS(=O)(=O)c3cnn(C(c4ccccc4)(c4ccccc4)c4ccccc4)c3)n2)cc1 ; CC(C)Cc1ccccc1-c1cc(Oc2ccc(C(C)(C)O)cc2)nc(NS(=O)(=O)c2cn[nH]c2)n1]

Stage 1: N-(4,6-dichloropyrimidin-2-yl)-1-methyl-pyrazole-4-sulfonamide, 2-(2-isobutylphenyl)-4,4,5,5-tetramethyl-1,3,2-dioxaborolane (approximately 29.92 mg, 0.1150 mmol), Pd(dppf)Cl₂ (approximately 5.610 mg, 0.007667 mmol) and cesium carbonate (approximately 74.94 mg, 0.2300 mmol) in DME (400 μL) and water (100 μL) were added and the mixture was purged with nitrogen for 5 minutes. The mixture was vigorously stirred under N2 at 100° C. for 1 hour as needed. The reaction mixture was cooled down to room temperature, filtered and purified by reverse phase HPLC (HCl modifier, 30-99% ACN-H₂O) to give the protected product.

Stage 2: Intermediate from Stage 1 was dissolved in DCM/TFA (approximately 8.742 mg, 5.907 μL, 0.07667 mmol), followed by addition of DCM (500 μL). The mixture was stirred at room temperature for 1 hour. The solvents was removed and the crudes was dissolved in DMSO, filtered, and purified on reverse phase HPLC (HCl modifier, 15-75% ACN-H₂O) to give N-[4-[4-(1-hydroxy-1-methyl-ethyl)phenoxy]-6-(2-isobutylphenyl)pyrimidin-2-yl]-1H-pyrazole-4-sulfonamide (2 mg, 5%). ¹H NMR (400 MHz, DMSO-d₆) δ 13.45 (s, 1H), 11.61 (s, 1H), 7.66 (s, 1H), 7.64-7.57 (m, 2H), 7.50 (s, 1H), 7.41 (s, 2H), 7.37-7.27 (m, 3H), 7.26-7.16 (m, 1H), 6.66 (s, 1H), 5.12 (s, 1H), 1.47 (s, 6H), 1.24 (s, 1H), 0.70 (d, J=6.6 Hz, 6H). ESI-MS m/z calc. 507.19403, found 508.0 (M+1)⁺; Retention time: 1.69 minutes; LC method A.

### Example 109: Preparation of Compound 347

### Step 1: N-[4-(2,6-dimethylphenyl)-6-[4-(1-hydroxy-1-methyl-ethyl)phenoxy]pyrimidin-2-yl]-1H-pyrazole-4-sulfonamide (Compound 347)

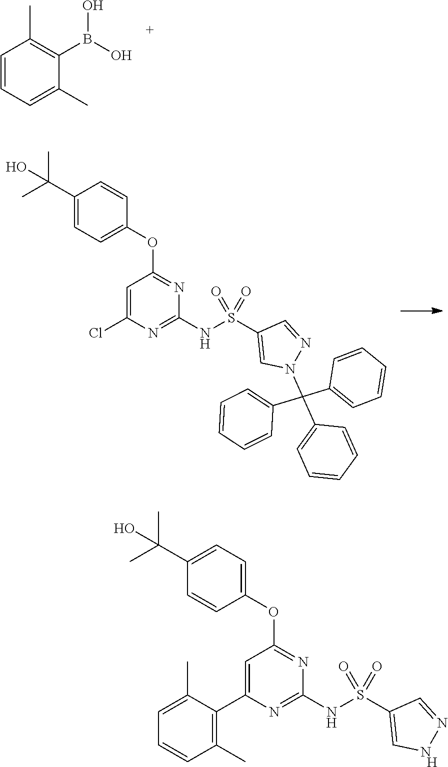

N-(4,6-dichloropyrimidin-2-yl)-1-methyl-pyrazole-4-sulfonamide, (2,6-dimethylphenyl)boronic acid (approximately 17.25 mg, 0.1150 mmol), Pd(dppf)C₁₂ (approximately 5.610 mg, 0.007667 mmol) and Cesium carbonate (approximately 74.94 mg, 0.2300 mmol) in DME (400 μL) and water (100 μL) were added and the mixture was purged with nitrogen for 5 minutes. The mixture was vigorously stirred under nitrogen at 100° C. for 1 hour as needed. The reaction mixture was cooled down to room temperature, filtered and purified on reverse phase HPLC (HCl modifier, 30-99% ACN-H₂O) to give the protected product. The intermediates were dissolved in DCM/TFA (approximately 8.742 mg, 5.907 μL, 0.07667 mmol), followed by addition of DCM (500 μL). The mixture was stirred at room temperature for 1 hour. The solvents were removed and the crude was dissolved in DMSO, filtered, and purified on reverse phase HPLC (HCl modifier, 15-75% ACN-H₂O) to give. N-[4-(2,6-dimethylphenyl)-6-[4-(1-hydroxy-1-methyl-ethyl)phenoxy]pyrimidin-2-yl]-1H-pyrazole-4-sulfonamide (2.1 mg, 6%). ESI-MS m/z calc. 479.16272, found 480.0 (M+1)⁺; Retention time: 1.42 minutes; LC method A.

### Example 110: Preparation of Compound 348

### Step 1: N-[4-[4-(1-hydroxy-1-methyl-ethyl)phenoxy]-6-(2-isopropylphenyl)pyrimidin-2-yl]-1H-pyrazole-4-sulfonamide (Compound 348)

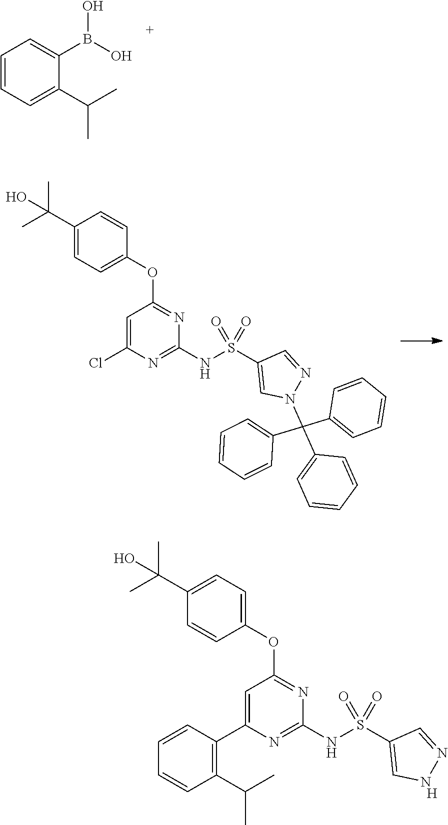

N-(4,6-dichloropyrimidin-2-yl)-1-methyl-pyrazole-4-sulfonamide, (2-isopropylphenyl)boronic acid (approximately 18.86 mg, 0.1150 mmol), Pd(dppf)C₁₂ (approximately 5.610 mg, 0.007667 mmol) and Cesium carbonate (approximately 74.94 mg, 0.2300 mmol) in DME (400 μL) and water (100 μL) were added and the mixture was purged with nitrogen for 5 minutes. The mixture was vigorously stirred under nitrogen at 100° C. for 1 hour as needed. The reaction mixture was cooled down to room temperature, filtered and purified on reverse phase HPLC (HCl modifier, 30-99% ACN-H₂O) to give protected products. The intermediates were dissolved in DCM/TFA (approximately 8.742 mg, 5.907 μL, 0.07667 mmol), followed by addition of DCM (500 μL). The mixture was stirred at room temperature for 1 hour. The solvents was removed and the crude was dissolved in DMSO, filtered, and purified on reverse phase HPLC (HCl modifier, 15-75% ACN-H₂O) to give N-[4-[4-(1-hydroxy-1 -methyl-ethyl)phenoxy]-6-(2-isopropylphenyl)pyrimidin-2-yl]-1H-pyrazole-4-sulfonamide (2.1 mg, 6%). ESI-MS m/z calc. 493.17838, found 494.0 (M+1)⁺; Retention time: 16.0 minutes; LC method A.

### Example 111: Preparation of Compound 349

### Step 1: N-[4-[isopentyl(methyl)amino]-6-phenoxy-pyrimidin-2-yl]benzenesulfonamide (Compound 349)

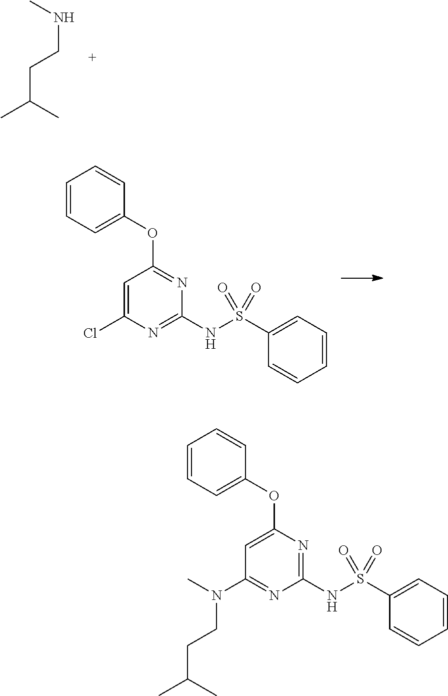

A solution of N-(4-chloro-6-phenoxy-pyrimidin-2-yl)benzenesulfonamide (27.14 mg, 0.075 mmol), N,3-dimethylbutan-1-amine (22.77 mg, 0.225 mmol) and DIPEA (48.47 mg, 65.32 μL, 0.375 mmol) in DMSO (0.4 mL) was heated at 100° C. for 16 hours. The reaction mixture was diluted with MeOH, filtered and purification by HPLC (1-99% ACN in water (HCl modifier)) to give N-[4-[isopentyl(methyl)amino]-6-phenoxy-pyrimidin-2-yl]benzenesulfonamide (19.8 mg, 62%). ESI-MS m/z calc. 426.17255, found 427.5 (M+1)⁺; Retention time: 2.03 minutes; LC method A.

### Example 112: Preparation of Compound 350 and Compound 351

### Step 1: N-[4-phenoxy-6-(1-piperidyl)pyrimidin-2-yl]benzenesulfonamide (Compound 350) and N-[4,6-bis(1-piperidyl)pyrimidin-2-yl]benzenesulfonamide (Compound 351)

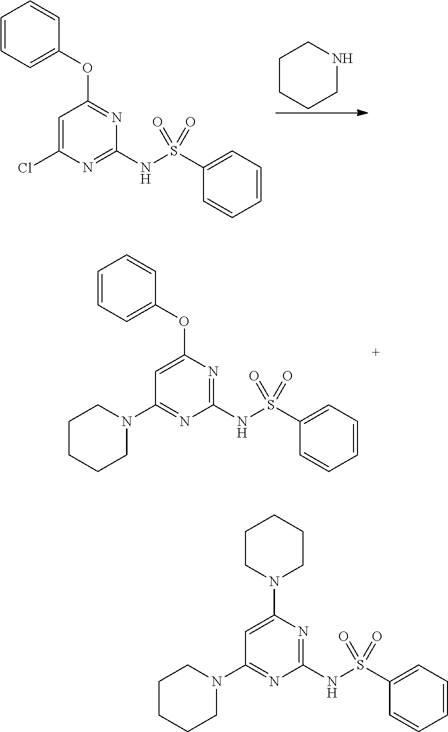 [SMILES: O=S(=O)(Nc1nc(Cl)cc(Oc2ccccc2)n1)c1ccccc1 ; C1CCNCC1 ; O=S(=O)(Nc1nc(Oc2ccccc2)cc(N2CCCCC2)n1)c1ccccc1 ; O=S(=O)(Nc1nc(N2CCCCC2)cc(N2CCCCC2)n1)c1ccccc1]

A solution of N-(4-chloro-6-phenoxy-pyrimidin-2-yl)benzenesulfonamide (27.14 mg, 0.075 mmol), piperidine (19.16 mg, 22.25 μL, 0.225 mmol) and DIPEA (48.47 mg, 65.32 μL, 0.375 mmol) in DMSO (0.4 mL) was heated at 100° C. for 16 hours. The reaction mixture was diluted with MeOH, filtered and purification by HPLC (1-99% ACN in water (HCl modifier)) to give N-[4-phenoxy-6-(1-piperidyl)pyrimidin-2-yl]benzenesulfonamide (6.6 mg, 21%). ESI-MS m/z calc. 410.14127, found 411.5 (M+1)⁺; Retention time: 1.84 minutes; LC method A and N-[4,6-bis(1-piperidyl)pyrimidin-2-yl]benzenesulfonamide (6.8 mg). ESI-MS m/z calc. 401.18854, found 402.5 (M+1)⁺; Retention time: 1.49 minutes; LC method A.

### Example 113: Preparation of Compound 352

### Step 1: 1-Methyl-N-[4-(4-piperazin-1-ylphenoxy)-6-(2-sec-butylphenoxy)pyrimidin-2-yl]pyrazole-4-sulfonamide

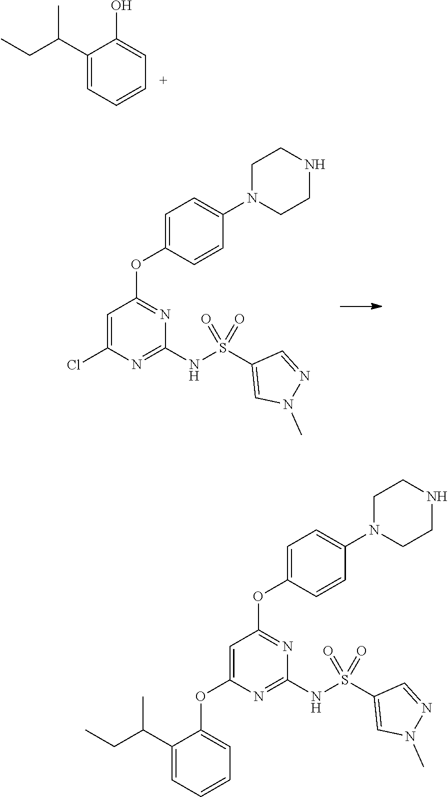 [SMILES: CCC(C)c1ccccc1O ; Cn1cc(S(=O)(=O)Nc2nc(Cl)cc(Oc3ccc(N4CCNCC4)cc3)n2)cn1 ; CCC(C)c1ccccc1Oc1cc(Oc2ccc(N3CCNCC3)cc2)nc(NS(=O)(=O)c2cnn(C)c2)n1]

A heterogeneous solution consisting of 2-sec-butylphenol (approximately 20.04 mg 0.1334 mmol), N-[4-chloro-6-(4-piperazin-1-ylphenoxy)pyrimidin-2-yl]-1-methyl-pyrazole-4-sulfonamide (20 mg, 0.04445 mmol), and cesium carbonate in NMP (0.178 mL) was heated in a sealed vial to 120° C. for 16 hours. The reaction was diluted with DMSO (0.5 mL) and acidified with hydrochloric acid. The solution was purified by HPLC (acetonitrile in water with 0.1% hydrochloric acid) to give 1-methyl-N-[4-(4-piperazin-1-ylphenoxy)-6-(2-sec-butylphenoxy)pyrimidin-2-yl]pyrazole-4-sulfonamide (3.5 mg, 14%). ESI-MS m/z calc. 563.23145, found 564.58 (M+1)⁺; Retention time: 1.53 minutes; LC method A.

### Example 114: Preparation of Compound 353

### Step 1: 1-Methyl-N-[4-(4-piperazin-1-ylphenoxy)-6-(2,4,6-trimethylphenoxy)pyrimidin-2-yl]pyrazole-4-sulfonamide (Compound 353)

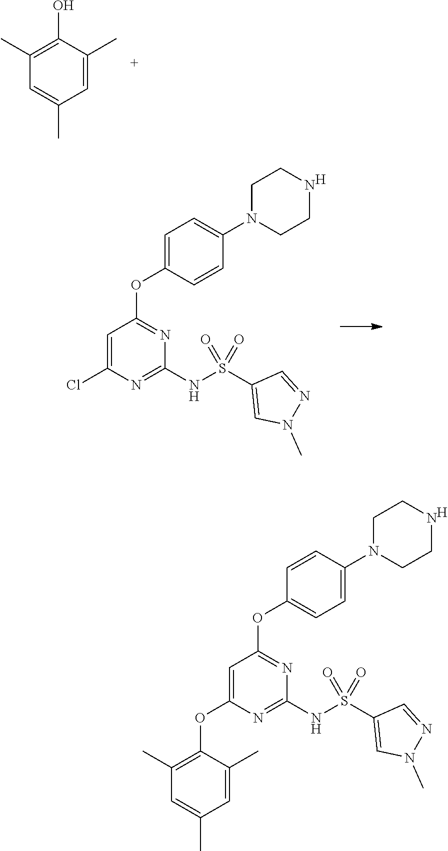

A heterogeneous solution consisting of 2,4,6-trimethylphenol (approximately 18.17 mg, 0.1334 mmol), N-[4-chloro-6-(4-piperazin-1-ylphenoxy)pyrimidin-2-yl]-1-methyl-pyrazole-4-sulfonamide (20 mg, 0.04445 mmol), and cesium carbonate in NMP (0.178 mL) was heated in a sealed vial to 120° C. for 16 hours. The reaction was diluted with DMSO (0.5 mL) and acidified with hydrochloric acid. The solution was purified by HPLC (acetonitrile in water with 0.1% hydrochloric acid) to give 1-methyl-N-[4-(4-piperazin-1-ylphenoxy)-6-(2,4,6-trimethylphenoxy)pyrimidin-2-yl]pyrazole-4-sulfonamide (10.6 mg, 390%). ESI-MS m/z calc. 549.2158, found 550.57 (M+1)⁺; Retention time: 1.48 minutes; LC method A.

### Example 115: Characterization of New Compounds

The compounds in the following tables were prepared in a manner analogous to that described above using commercially available reagents and intermediates described herein.

| | | LCMS | | | |
|---|---|---|---|---|---|
| | | Room | | | |
| Compound | | Temperature | Calc. | | LCMS |
| number | Structure | (min) | mass | M + 1 | Method |
| 354 | 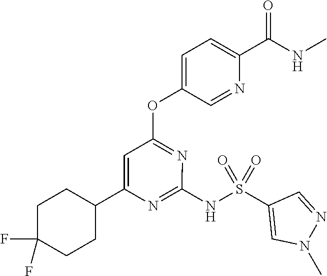 [SMILES: CNC(=O)c1ccc(Oc2cc(C3CCC(F)(F)CC3)nc(NS(=O)(=O)c3cnn(C)c3)n2)cn1] | 1.33 | 507.15 | 507.68 | A |
| 355 | 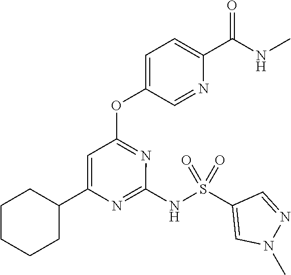 [SMILES: CNC(=O)c1ccc(Oc2cc(C3CCCCC3)nc(NS(=O)(=O)c3cnn(C)c3)n2)cn1] | 1.33 | 471.169 | 471.8 | A |
| 356 | 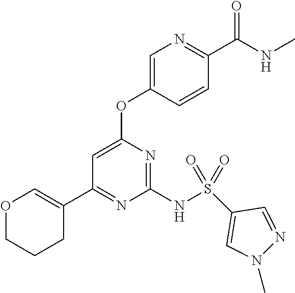 [SMILES: CNC(=O)c1ccc(Oc2cc(C3=COCCC3)nc(NS(=O)(=O)c3cnn(C)c3)n2)cn1] | 1.33 | 471.132 | 472.42 | A |
| 357 | 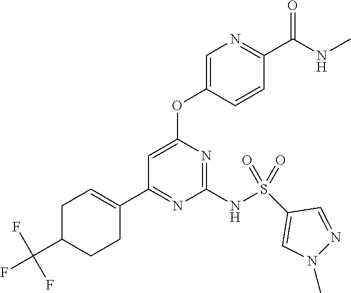 [SMILES: CNC(=O)c1ccc(Oc2cc(C3=CCC(C(F)(F)F)CC3)nc(NS(=O)(=O)c3cnn(C)c3)n2)cn1] | 1.65 | 537.141 | 538.46 | A |
| 358 | 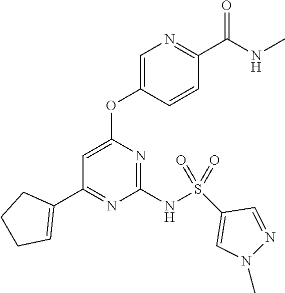 [SMILES: CNC(=O)c1ccc(Oc2cc(C3=CCCC3)nc(NS(=O)(=O)c3cnn(C)c3)n2)cn1] | 1.45 | 455.138 | 456.42 | A |
| 344 | 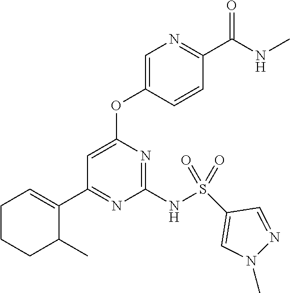 [SMILES: CNC(=O)c1ccc(Oc2cc(C3=CCCCC3C)nc(NS(=O)(=O)c3cnn(C)c3)n2)cn1] | 1.55 | 483.169 | 484.46 | A |
| 359 | 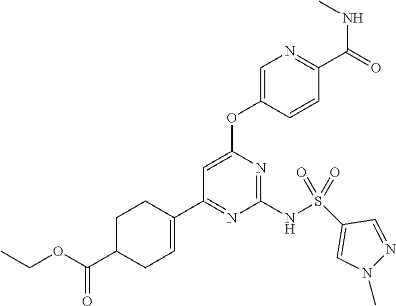 [SMILES: CCOC(=O)C1CC=C(c2cc(Oc3ccc(C(=O)NC)nc3)nc(NS(=O)(=O)c3cnn(C)c3)n2)CC1] | 1.39 | 541.174 | 542.48 | A |
| 360 | 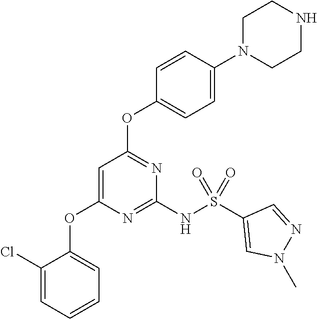 [SMILES: Cn1cc(S(=O)(=O)Nc2nc(Oc3ccc(N4CCNCC4)cc3)cc(Oc3ccccc3Cl)n2)cn1] | 1.29 | 541.13 | 542.48 | A |
| 361 | 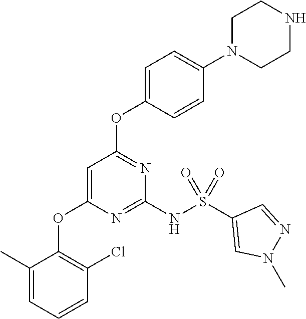 [SMILES: Cc1cccc(Cl)c1Oc1cc(Oc2ccc(N3CCNCC3)cc2)nc(NS(=O)(=O)c2cnn(C)c2)n1] | 1.37 | 555.146 | 556.49 | A |
| 362 | 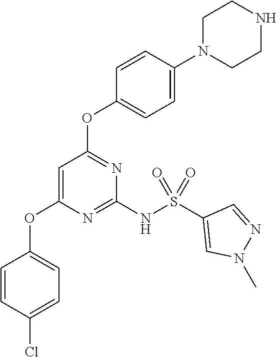 [SMILES: Cn1cc(S(=O)(=O)Nc2nc(Oc3ccc(Cl)cc3)cc(Oc3ccc(N4CCNCC4)cc3)n2)cn1] | 1.36 | 541.13 | 542.45 | A |
| 363 | 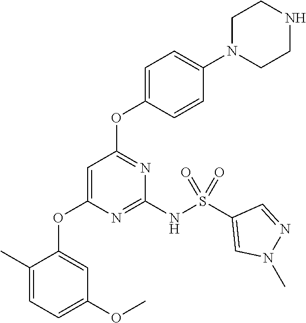 [SMILES: COc1ccc(C)c(Oc2cc(Oc3ccc(N4CCNCC4)cc3)nc(NS(=O)(=O)c3cnn(C)c3)n2)c1] | 1.32 | 551.195 | 552.53 | A |
| 364 | 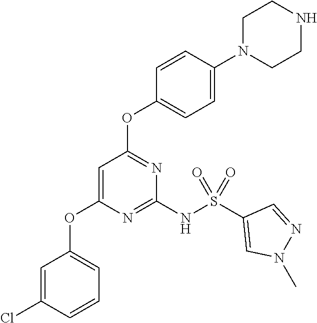 [SMILES: Cn1cc(S(=O)(=O)Nc2nc(Oc3ccc(N4CCNCC4)cc3)cc(Oc3cccc(Cl)c3)n2)cn1] | 1.36 | 541.13 | 542.48 | A |
| 365 | 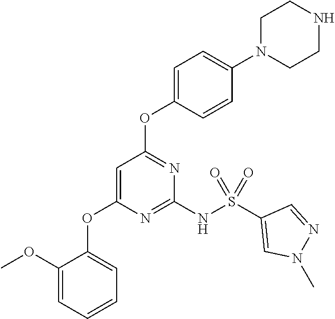 [SMILES: COc1ccccc1Oc1cc(Oc2ccc(N3CCNCC3)cc2)nc(NS(=O)(=O)c2cnn(C)c2)n1] | 1.21 | 537.179 | 538.53 | A |
| 366 | 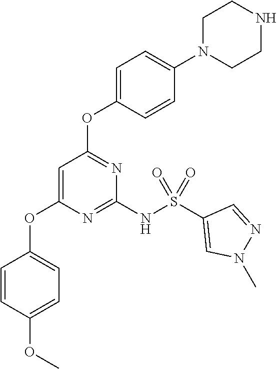 [SMILES: COc1ccc(Oc2cc(Oc3ccc(N4CCNCC4)cc3)nc(NS(=O)(=O)c3cnn(C)c3)n2)cc1] | 1.23 | 537.179 | 538.53 | A |
| 367 | 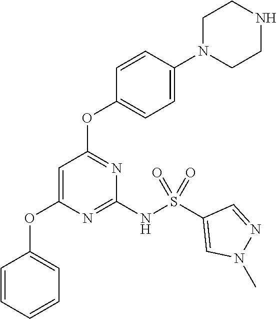 [SMILES: Cn1cc(S(=O)(=O)Nc2nc(Oc3ccccc3)cc(Oc3ccc(N4CCNCC4)cc3)n2)cn1] | 1.22 | 507.169 | 508.52 | A |
| 368 | 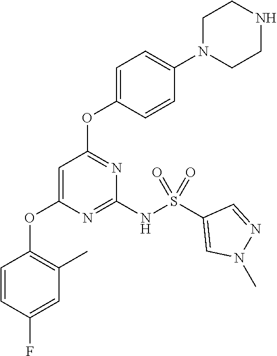 [SMILES: Cc1cc(F)ccc1Oc1cc(Oc2ccc(N3CCNCC3)cc2)nc(NS(=O)(=O)c2cnn(C)c2)n1] | 1.32 | 539.175 | 540.52 | A |
| 369 | 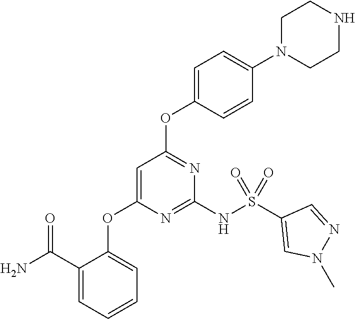 [SMILES: Cn1cc(S(=O)(=O)Nc2nc(Oc3ccc(N4CCNCC4)cc3)cc(Oc3ccccc3C(N)=O)n2)cn1] | 1.15 | 550.175 | 551.52 | A |
| 370 | 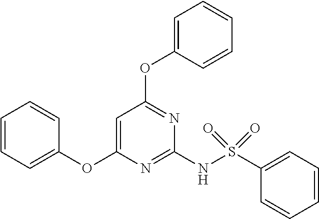 [SMILES: O=S(=O)(Nc1nc(Oc2ccccc2)cc(Oc2ccccc2)n1)c1ccccc1] | 1.97 | 419.094 | 420.25 | A |
| 371 | 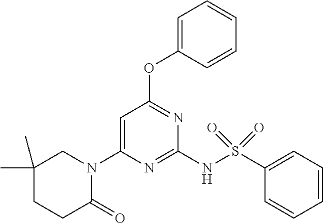 [SMILES: CC1(C)CCC(=O)N(c2cc(Oc3ccccc3)nc(NS(=O)(=O)c3ccccc3)n2)C1] | 1.77 | 452.152 | 453.4 | A |
| 372 | 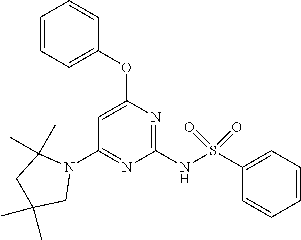 [SMILES: CC1(C)CN(c2cc(Oc3ccccc3)nc(NS(=O)(=O)c3ccccc3)n2)C(C)(C)C1] | 2.15 | 452.188 | 453.5 | A |
| 373 | 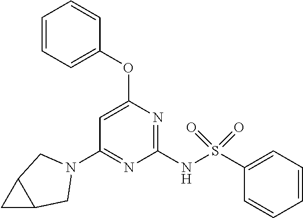 [SMILES: O=S(=O)(Nc1nc(Oc2ccccc2)cc(N2CC3CC3C2)n1)c1ccccc1] | 1.77 | 408.126 | 409.5 | A |
| 374 | 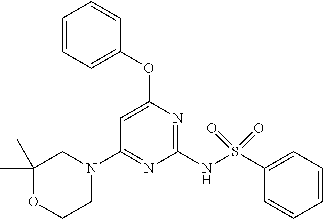 [SMILES: CC1(C)CN(c2cc(Oc3ccccc3)nc(NS(=O)(=O)c3ccccc3)n2)CCO1] | 1.66 | 440.152 | 441.5 | A |
| 375 | 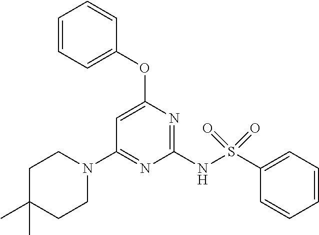 [SMILES: CC1(C)CCN(c2cc(Oc3ccccc3)nc(NS(=O)(=O)c3ccccc3)n2)CC1] | 2.04 | 438.173 | 439.5 | A |
| 376 | 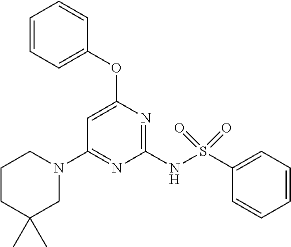 [SMILES: CC1(C)CCCN(c2cc(Oc3ccccc3)nc(NS(=O)(=O)c3ccccc3)n2)C1] | 2.01 | 438.173 | 439.5 | A |
| 377 | 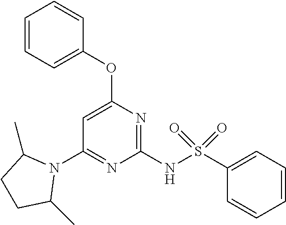 [SMILES: CC1CCC(C)N1c1cc(Oc2ccccc2)nc(NS(=O)(=O)c2ccccc2)n1] | 1.94 | 424.157 | 425.5 | A |
| 378 | 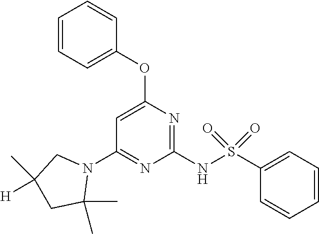 [SMILES: CC1CN(c2cc(Oc3ccccc3)nc(NS(=O)(=O)c3ccccc3)n2)C(C)(C)C1] | 2.08 | 438.173 | 439.5 | A |
| 379 | 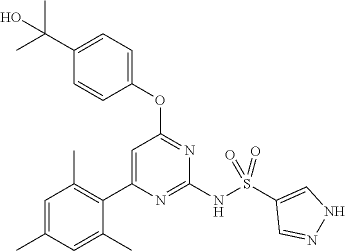 [SMILES: Cc1cc(C)c(-c2cc(Oc3ccc(C(C)(C)O)cc3)nc(NS(=O)(=O)c3cn[nH]c3)n2)c(C)c1] | 1.54 | 493.178 | 494 | A |
| 380 | 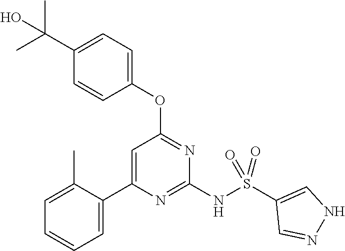 [SMILES: Cc1ccccc1-c1cc(Oc2ccc(C(C)(C)O)cc2)nc(NS(=O)(=O)c2cn[nH]c2)n1] | 1.41 | 465.147 | 466 | A |
| 381 | 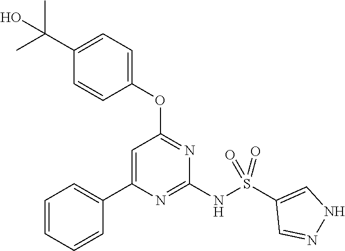 [SMILES: CC(C)(O)c1ccc(Oc2cc(-c3ccccc3)nc(NS(=O)(=O)c3cn[nH]c3)n2)cc1] | 1.41 | 451.131 | 452 | A |

| Compound | |
|---|---|
| number | NMR |
| 381 | ¹H NMR (400 MHz, DMSO-d₆) δ 13.44 (s, 1H), 11.55 (s, 1H), 8.12 (dt, J = 7.4, |
| | 3.9 Hz, 2H), 7.72 − 7.51 (m, 7H), 7.25 (s, 1H), 7.23 − 7.15 (m, 2H), 2.54 (s, 17H), |
| | 1.47 (s, 6H). |

### Example 116: Preparation of Compound 382

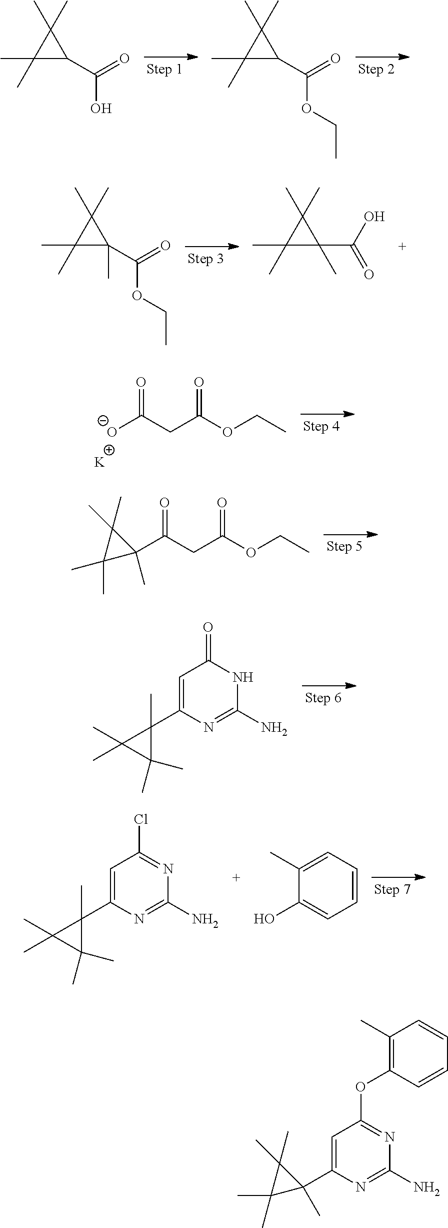

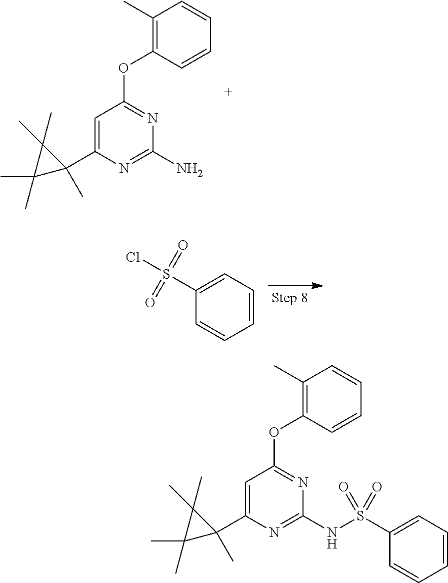

### Step 1: Ethyl 2,2,3,3-tetramethylcyclopropanecarboxylate

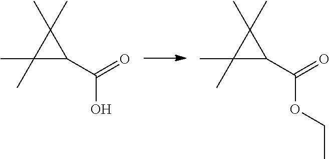

2,2,3,3-Tetramethylcyclopropanecarboxylic acid (15 g, 105.5 mmol) was dissolved in absolute ethanol (100 mL) with sulfuric acid (300 uL) then refluxed 48 hours. The reaction mixture was concentrated under reduced pressure to provide the desired product ethyl 2,2,3,3-tetramethylcyclopropanecarboxylate (14.71 g, 81%) as a clear liquid. ¹H NMR (300 MHz, CDCl₃) 4.07 (q, J=7.0 Hz, 2H), 1.24 (t, J=7.2 Hz, 3H), 1.23 (s, 6H), 1.17 (s, 6H), 1.16 (s, 1H).

### Step 2: Ethyl 1,2,2,3,3-pentamethylcyclopropanecarboxylate

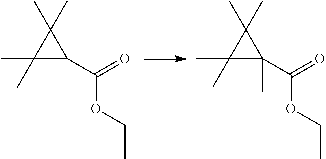

Diisopropylamine (7.5 mL,53.5 mmol, distilled over calcium hydride) was dissolved in anhydrous THE (60 mL). The reaction mixture kept under nitrogen and cooled to −15° C. BuLi (2.5 M in hexanes, 19.5 mL, 48.8 mmol) was slowly added while stirring and maintaining the temperature at −15° C. The resulting mixture was stirred for additional 10 minutes, allowed to warm up to 0° C., following by stirring for an additional 10 minutes. The reaction mixture cooled to −15° C., ethyl 2,2,3,3-tetramethylcyclopropanecarboxylate (7.46 g, 43.8 mmol) was added dropwise, and the mixture was stirred for 40 min at −15° C. The temperature was elevated to ˜8° C., and iodomethane (4 mL, 64.3 mmol) was added to the reaction mixture. The reaction mixture was stirred for an additional 30 minutes. The organic solvent was removed under reduced pressure and the oily residue dispersed in ethyl acetate (40 mL), washed with water (20 mL), brine (10 mL), dried over Na₂SO₄, and filtered. The solvent was evaporated to yield ethyl 1,2,2,3,3-pentamethylcyclopropanecarboxylate as yellow oil (7.0 g, 95% purity, 82% yield). ¹H NMR (300 MHz, CDCl₃) δ 4.10 (q, J=7.1 Hz, 2H), 1.26 (s, 3H), 1.24 (t, J=7.1 Hz, 3H), 1.12 (s, 6H), 1.01 (s, 6H).

### Step 3: 1,2,2,3,3-Pentamethylcyclopropanecarboxylic acid

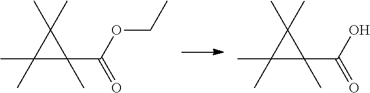

Ethyl 1,2,2,3,3-pentamethylcyclopropanecarboxylate (11.5 g, 62.4 mmol) was dissolved in ethanol (100 mL). Water (20 mL) was added followed by KOH (14.0 g, 249.6 mmol). The mixture was stirred at 90° C. for two days. KOH (7.0 g, 125 mmol) was added and the reaction was continued for another 24 hours. The reaction was cooled down and then concentrated under reduced pressure. The residue was dissolved in water (40 mL), extracted with MTBE (30 mL×2). The aqueous layer was acidified with 6 N HCl to pH 3-4, extracted with ethyl acetate (100 mL×2). The organic layer was washed with brine (50 mL), dried over anhydrous sodium sulfate and concentrated under reduced pressure to afford 1,2,2,3,3-pentamethylcyclopropanecarboxylic acid as a yellow solid (8.6 g, 88% yield). ¹H NMR (300 MHz, CDCl₃) δ 1.28 (s, 3H), 1.21 (s, 6H), 1.05 (s, 6H). ESI-MS m/z calc. 156.11504, found 155.1 (M−1)⁺; Retention time: 1.85 minutes; LC method C.

### Step 4: Ethyl 3-oxo-3-(1,2,2,3,3-pentamethylcyclopropyl)propanoate

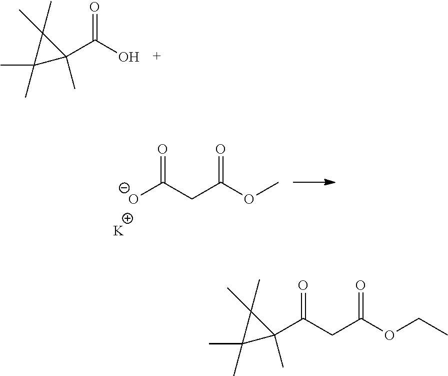

A solution of 1,2,2,3,3-pentamethylcyclopropanecarboxylic acid (3.12 g, 20.0 mmol) in THE (50 mL) was treated with carbonyl diimidazole (3.43 g, 21.2 mmol) and left to stir at room temperature for 2 hours. Added magnesium chloride (2.09 g, 22.0 mmol) and ethyl potassium malonate (3.74 g, 22.0 mmol) and the reaction was heated in an oil bath at 50° C. overnight. Once cooled to room temperature, the reaction mixture was transferred to a 500-mL separatory funnel with methyl tert-butyl ether (300 mL) and washed with 1 N HCl (2×100 mL). The organic layer was then washed with water (100 mL), brine (100 mL), dried over sodium sulfate, filtered and concentrated under reduced pressure. The residue was purified by silica gel chromatography on a 40-g column (Biotage SP1), eluting from 0% to 20% ethyl acetate in heptanes, to afford ethyl 3-oxo-3-(1,2,2,3,3-pentamethylcyclopropyl)propanoate (480 mg, 11% yield) as a pale orange oil. ESI-MS m/z calc. 226.15689, found 227.2 (M+1)⁺; Retention time: 2.21 minutes; LC method C.

### Step 5: 2-Amino-4-(1,2,2,3,3-pentamethylcyclopropyl)-1H-pyrimidin-6-one

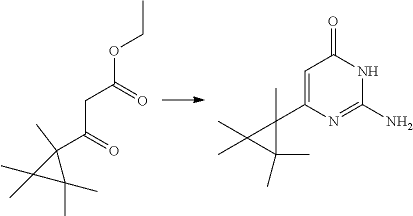 [SMILES: CCOC(=O)CC(=O)C1(C)C(C)(C)C1(C)C ; CC1(C)C(C)(C)C1(C)c1cc(=O)[nH]c(N)n1]

A flame-dried 50-mL flask was charged with anhydrous ethanol (6.0 mL) and sodium metal (95 mg, 4.1 mmol, pre-washed with heptanes) was added. Once gas evolution had stopped and all the sodium had reacted, guanidine hydrochloride (374 mg, 3.92 mmol) was added (note: a milky white suspension appears). After 5 minutes, a solution of ethyl 3-oxo-3-(1,2,2,3,3-pentamethylcyclopropyl)propanoate (571 mg, 2.52 mmol) in anhydrous ethanol (1.5 mL, +1.0 mL rinse) was added and the reaction was heated in an oil bath at 80° C. for 21 hours. Once cooled, the solvent was removed under reduced pressure, suspended in water (about 15 mL) and acidified to a pH of 2-3 with concentrated HCl. The mixture was transferred to a separatory funnel and extracted with a mixture of isopropanol in chloroform (1:2, 3×20 mL). The combined organic layers were dried over sodium sulfate, filtered and concentrated under reduced pressure to afford a slightly gummy solid. This solid was triturated with methyl tert-butyl ether (about 15 mL), washed with additional methyl tert-butyl ether and dried under high vacuum to afford 2-amino-4-(1,2,2,3,3-pentamethylcyclopropyl)-1H-pyrimidin-6-one (142 mg, 25% yield) as a white solid. ¹H NMR (300 MHz, DMSO-d₆) 7.15 (br.s, 2H), 5.42 (s, 1H), 1.19 (s, 3H), 1.04 (s, 6H), 0.99 (s, 6H). ESI-MS m/z calc. 221.15282, found 222.2 (M+1)⁺; Retention time: 1.28 minutes; LC method C.

### Step 6: 4-Chloro-6-(1,2,2,3,3-pentamethylcyclopropyl)pyrimidin-2-amine

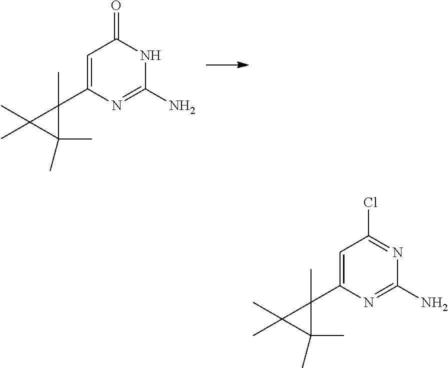 [SMILES: CC1(C)C(C)(C)C1(C)c1cc(=O)[nH]c(N)n1 ; CC1(C)C(C)(C)C1(C)c1cc(Cl)nc(N)n1]

A suspension of 2-amino-4-(1,2,2,3,3-pentamethylcyclopropyl)-1H-pyrimidin-6-one (142 mg, 0.642 mmol) in dioxane (2.4 mL) and phosphorus oxychloride (0.60 mL, 6.4 mmol) was gradually heated up to 80° C. in an oil bath (upon heating, turns to a pale yellow solution). After heating for 3.5 hours the reaction was removed from the oil bath and left to cool to room temperature. The reaction was quenched by adding portionwise to 5% aqueous sodium bicarbonate (50 mL) cooled in an ice bath. The aqueous layer was then transferred to a 125-mL separatory funnel and extracted with dichloromethane (3×20 mL). The combined organic layers were dried over sodium sulfate, filtered and concentrated under reduced pressure. The residue was purified by silica gel chromatography on a 24-g column (Biotage SP1), eluting from 0% to 30% ethyl acetate in heptanes, to afford 4-chloro-6-(1,2,2,3,3-pentamethylcyclopropyl)pyrimidin-2-amine (96 mg, 62% yield) as a white solid. ¹H NMR (300 MHz, CDCl₃) 6.44 (s, 1H), 5.31 (br s, 2H), 1.24 (s, 3H), 1.12 (s, 6H), 0.97 (s, 6H). ESI-MS m z calc. 239.11893, found 240.2 (M+1)⁺; Retention time: 2.97 minutes; LC method H.

### Step 7: 4-(2-Methylphenoxy)-6-(1,2,2,3,3-pentamethylcyclopropyl)pyrimidin-2-amine

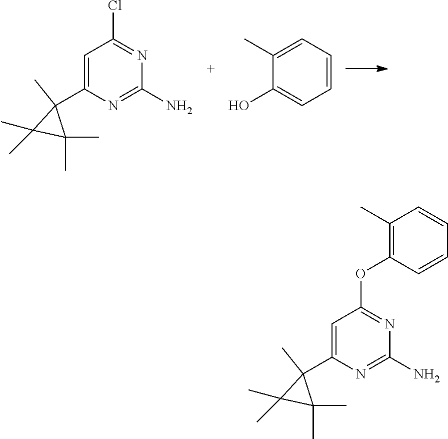

NaH (41.8 mg of 60% w/w, 1.05 mmol) was added to o-cresol (78 mg, 0.72 mmol) in NMP (500 μL) at 0° C. The mixture was stirred for 45 minutes. then added to 4-chloro-6-(1,2,2,3,3-pentamethylcyclopropyl)pyrimidin-2-amine (77 mg, 0.3212 mmol) in NMP (500 μL). The resulting mixture was stirred at 100° C. for 20 hours. The reaction mixture was cooled down, filtered, and purified on reverse phase HPLC (HCl modifier, 10-60% ACN-H₂O) to give 4-(2-methylphenoxy)-6-(1,2,2,3,3-pentamethylcyclopropyl)pyrimidin-2-amine (45.2 mg, 43%) ESI-MS m/z calc. 311.19977, found 312.0 (M+1)⁺; Retention time: 1.39 minutes; LC method A.

### Step 8: N-[4-(2-methylphenoxy)-6-(1,2,2,3,3-pentamethylcyclopropyl)pyrimidin-2-yl]benzenesulfonamide (Compound 382)

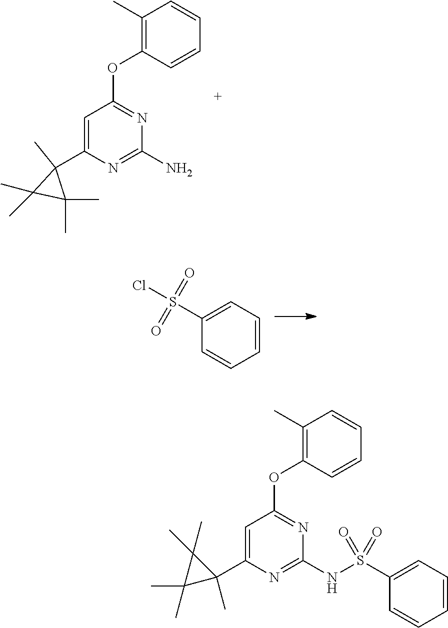 [SMILES: Cc1ccccc1Oc1cc(C2(C)C(C)(C)C2(C)C)nc(N)n1 ; O=S(=O)(Cl)c1ccccc1 ; Cc1ccccc1Oc1cc(C2(C)C(C)(C)C2(C)C)nc(NS(=O)(=O)c2ccccc2)n1]

To a solution of 4-(2-methylphenoxy)-6-(1,2,2,3,3-pentamethylcyclopropyl)pyrimidin-2-amine (17.8 mg, 0.055 mmol) in DMF (500 μL) at 0° C. was added NaH (14 mg of 60% w/w, 0.35 mmol) and the reaction mixture was stirred at this temperature for 5 minutes. the reaction mixture was removed from the cooling bath and stirred at room temperature for 20 minutes. benzenesulfonyl chloride (9 μL, 0.07052 mmol) was added slowly to the previous mixture and the resulting mixture was stirred at room temperature for 1 hours. LC/MS showed trace of product and starting material. The reaction mixture was stirred at 100° C. for 50 minutes. The reaction mixture was filtered and purified on reverse phase HPLC (HCl modifier, 20-80% ACN-H₂O) to give N-[4-(2-methylphenoxy)-6-(1,2,2,3,3-pentamethylcyclopropyl)pyrimidin-2-yl]benzenesulfonamide (2.5 mg, 10%) ¹H NMR (400 MHz, DMSO-d₆) δ 12.97 (s, 1H), 7.63-7.33 (m, 5H), 7.21-7.05 (m, 4H), 6.27 (s, 1H), 2.04 (s, 3H), 1.22-0.89 (m, 15H). ESI-MS m/z calc. 451.19296, found 452.0 (M+1)⁺; Retention time: 2.03 minutes; LC method A.

### Example 117: Preparation of Compound 383 and Compound 384

### Step 1: tert-Butyl 4-[4-[2-amino-6-(2,2,3,3-tetramethylcyclopropyl)pyrimidin-4-yl]oxyphenyl]piperazine-1-carboxylate

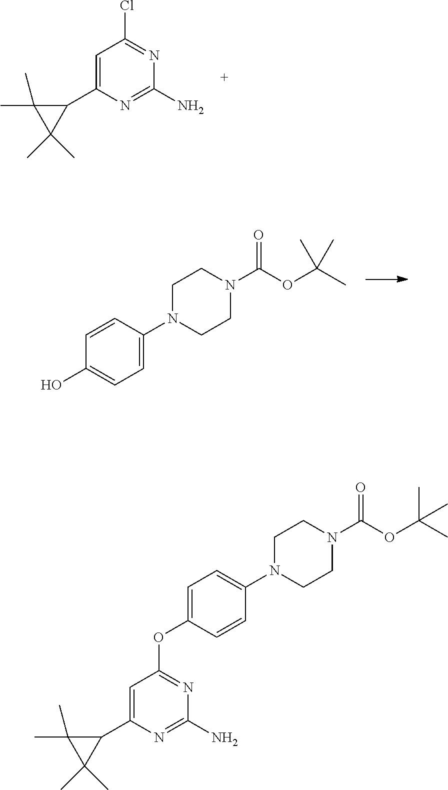

A mixture of 4-chloro-6-(2,2,3,3-tetramethylcyclopropyl)pyrimidin-2-amine (203.6 mg, 0.9020 mmol), cesium carbonate (617.3 mg, 1.895 mmol), and tert-butyl 4-(4-hydroxyphenyl)piperazine-1-carboxylate (372 mg, 1.336 mmol) was heated to 110° C. for 16 hours and then diluted with ethyl acetate (10 mL) and water (20 mL). The pH of aqueous layer is adjusted to 7-8 by the addition of 1 N HCl. Extract product from aqueous layer with ethyl acetate (5 mL×2). Combine the organic layers and wash with water (5 m) and then dry over anhydrous sodium sulfate, filter, and concentrate in vacuo. The crude was purified by silica using a gradient of ethyl acetate and hexane. The product came out at ˜ 25% ethyl acetate: tert-butyl 4-[4-[2-amino-6-(2,2,3,3-tetramethylcyclopropyl)pyrimidin-4-yl]oxyphenyl]piperazine-1-carboxylate (219.5 mg, 50%) ¹H NMR (400 MHz, DMSO-d₆) δ 7.06-6.93 (m, 4H), 6.36 (s, 2H), 5.86 (s, 1H), 3.47 (t, J=5.0 Hz, 4H), 3.08 (t, J=5.1 Hz, 4H), 1.43 (s, 9H), 1.35 (s, 1H), 1.18 (s, 6H), 1.17 (s, 6H). ESI-MS m/z calc. 467.28964, found 468.0 (M+1)⁺; Retention time: 1.61 minutes; LC method A.

### Step 2: tert-Butyl 4-[4-[2-(benzenesulfonamido)-6-(2,2,3,3-tetramethylcyclopropyl)pyrimidin-4-yl]oxyphenyl]piperazine-1-carboxylate (Compound 383) and N-[4-(4-piperazin-1-ylphenoxy)-6-(2,2,3,3-tetramethylcyclopropyl)pyrimidin-2-yl]benzenesulfonamide (Compound 384)

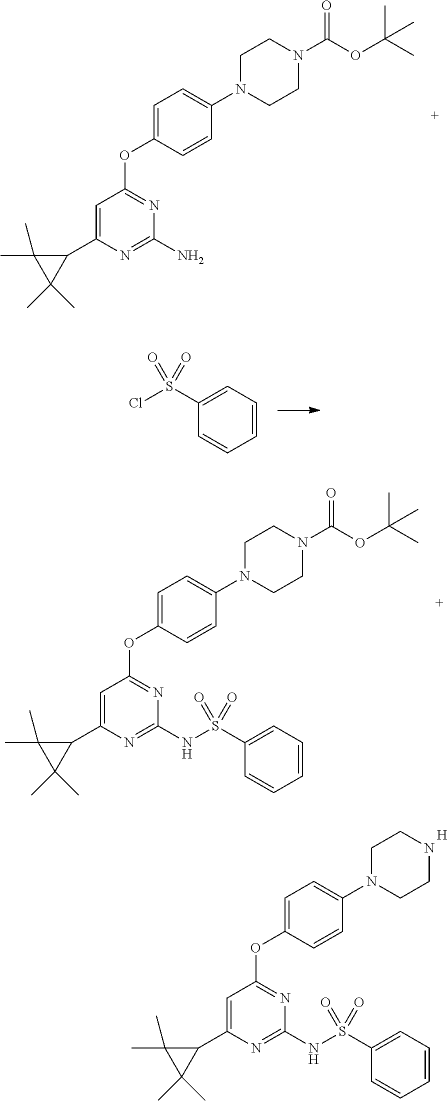

To a solution of tert-butyl 4-[4-[2-amino-6-(2,2,3,3-tetramethylcyclopropyl)pyrimidin-4-yl]oxyphenyl]piperazine-1-carboxylate (50 mg, 0.1069 mmol) in DMF (500.0 μL) at 0° C. was added NaH (approximately 21.38 mg of 60% w/w, 0.5345 mmol) and the reaction mixture was stirred at this temperature for 5 minutes, then removed from the cooling bath and stirred at room temperature for 10 minutes. benzenesulfonyl chloride (approximately 37.76 mg, 27.28 μL, 0.2138 mmol) was added slowly and the resulting mixture was stirred at room temperature for 1 hour. LC/MS showed 10% conversion. The reaction was let to go for 16 hours. at 70° C. The reaction mixture was filtered and purified on reverse phase HPLC (HCl modifier, 15-75% ACN-H₂O) to give tert-butyl 4-[4-[2-(benzenesulfonamido)-6-(2,2,3,3-tetramethylcyclopropyl)pyrimidin-4-yl]oxyphenyl]piperazine-1-carboxylate (1 mg, 2%) ESI-MS m/z calc. 607.28284, found 608.0 (M+1)⁺; Retention time: 2.16 minutes; LC method A.

TFA (500 μL, 6.490 mmol) was added to tert-butyl 4-[4-[2-(benzenesulfonamido)-6-(2,2,3,3-tetramethylcyclopropyl)pyrimidin-4-yl]oxyphenyl]piperazine-1-carboxylate (1 mg, 2%) in DCM (0.5 mL). The mixture was stirred for 30 minutes. at room temperature. Solvents were removed and the crude was dissolved in DMSO, filtered and purified on reverse phase HPLC (HCl modifier, 10-60% ACN-H₂O) to give N-[4-(4-piperazin-1-ylphenoxy)-6-(2,2,3,3-tetramethylcyclopropyl)pyrimidin-2-yl]benzenesulfonamide (6 mg, 11%) ¹H NMR (400 MHz, DMSO-d₆) δ 8.51 (s, 1H), 7.79 (td, J=6.2, 3.0 Hz, 2H), 7.76-7.66 (m, 2H), 7.04-6.90 (m, 4H), 6.36 (s, 2H), 5.84 (s, 1H), 3.18 (dd, J=6.5, 3.6 Hz, 4H), 3.06-3.00 (m, 4H), 1.33 (s, 1H), 1.16 (s, 6H), 1.16 (s, 6H). ESI-MS m/z calc. 507.2304, found 508.0 (M+1)⁺; Retention time: 1.45 minutes; LC method A.

### Example 118: Preparation of Compound 385

### Step 1: 2-Amino-6-tert-butyl-pyrimidin-4-ol

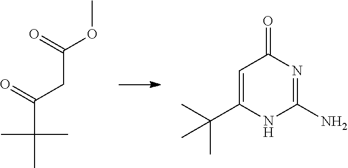

To a solution of methyl 4,4-dimethyl-3-oxo-pentanoate (25 mL, 156.5 mmol) and guanidine (hydrochloride salt) (approximately 17.94 g, 187.8 mmol) in methanol (500 mL) was added potassium tert-butoxide (approximately 87.81 g, 782.5 mmol) in portion over 45 minutes at ambient temperature with vigorous stirring. The reaction mixture was stirred at room temperature overnight. Reaction mixture was filtered, and the filtrate was concentrated until it became syrupy (˜⅔ of the volume was removed). The syrup was acidified to pH ˜5 with 6 N HCl. The white precipitate was filtered and dried in vacuum oven. 2-amino-6-tert-butyl-1H-pyrimidin-4-one (hydrochloride salt) (28.82 g, 90%) ¹H NMR (400 MHz, DMSO) δ 10.76 (s, 1H), 7.08 (s, 1H), 6.48 (s, 2H), 1.14 (s, 9H). ESI-MS m/z calc. 167.10587, found 168.0 (M+1)⁺; Retention time: 0.49 minutes; LC method A.

### Step 2: 4-tert-Butyl-6-chloro-pyrimidin-2-amine

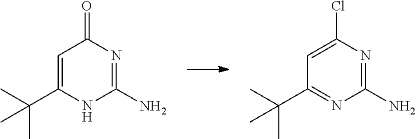

A mixture of 2-amino-6-tert-butyl-1H-pyrimidin-4-one (hydrochloride salt) (28.8 g, 140.0 mmol) in POCl₃ (100 mL, 1.073 mol) was stirred at 120° C. for 1 hour. The reaction mixture was cooled down to room temperature, filtered and the filtrate was concentrated until it became syrupy (˜⅔ volume was removed). Ice chips were added while the flask was swirled. The mixture turned cloudy when the volume was double. The mixture was let to sit for 30 minutes at room temperature, and the precipitate was filtered. The precipitate was washed with more H₂O to give 1.6 g of white solid (1^{st} batch, ˜95% purity). The filtrate was concentrated, and more precipitate came out of solution. The precipitate was filtered, washed with more H₂O to give 3.15 g (precipitate 3, ˜₇₅% purity). The resulting filtrate was neutralized with concentrate NH₄₀H and extracted with ethyl acetate (3×20 ml). The organic layer was dried over Na₂SO₄, concentrated, and purified by silica using a gradient of ethyl acetate/hexane. The product came out ˜ 10% ethyl acetate to give 3.58 g (column, 99.9% purity). Total amount: 4-tert-butyl-6-chloro-pyrimidin-2-amine (8.33 g, 32%) 1H NMR (400 MHz, DMSO) δ 6.96 (s, 2H), 6.64 (s, 1H), 1.22 (s, 9H). ESI-MS m/z calc. 185.07198, found 186.0 (M+1)⁺; Retention time: 1.19 minutes; LC method A.

### Step 3: N-(4-tert-butyl-6-phenoxy-pyrimidin-2-yl)benzenesulfonamide (Compound 385)

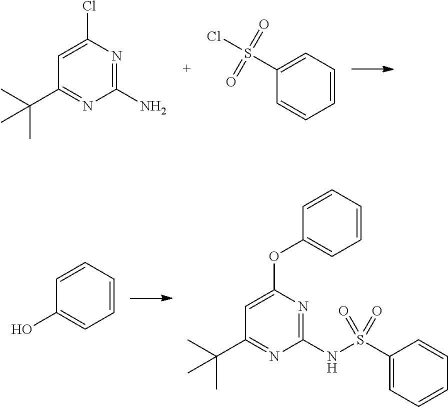 [SMILES: CC(C)(C)c1cc(Cl)nc(N)n1 ; O=S(=O)(Cl)c1ccccc1 ; Oc1ccccc1 ; CC(C)(C)c1cc(Oc2ccccc2)nc(NS(=O)(=O)c2ccccc2)n1]

To a solution of 4-tert-butyl-6-chloro-pyrimidin-2-amine (338.2 mg, 1.822 mmol) in DMF (5 mL) was added sodium hydride (approximately 94.75 mg of 60% w/w, 2.369 mmol) at 0° C. and stirred for 10 minutes at 0° C. To this mixture benzenesulfonyl chloride (approximately 386.1 mg, 279.0 μL, 2.186 mmol) was added dropwise and was stirred at 0° C. for 15 minutes. UPLC showed the formation of product with some starting material remained. The reaction mixture was quench with water and acidified with 1 N HCl, extracted with ethyl acetate (2×10ml). The organic layer was separated, dried over Na₂SO₄, concentrated and the residue was purified by silica. Product came out at ˜ 20% ethyl acetate/hexanes N-(4-tert-butyl-6-chloro-pyrimidin-2-yl)benzenesulfonamide (270 mg)¹H NMR (400 MHz, DMSO) δ 12.09 (s, 1H), 7.96 (d, J=8.5 Hz, 2H), 7.65 (d, J=5.8 Hz, 1H), 7.60 (t, J=7.3 Hz, 2H), 7.17 (s, 1H), 1.10 (s, 9H). ESI-MS m/z calc. 325.0652, found 326.0 (M+1)⁺; Retention time: 1.71 minutes; LC method A.

NaH (approximately 72.87 mg of 60% w/w, 1.822 mmol) was added to phenol (approximately 171.5 mg, 161.8 μL, 1.822 mmol) in DMF (1 mL) at rt. The mixture was stirred for 10 minutes. and N-(4-tert-butyl-6-chloro-pyrimidin-2-yl)benzenesulfonamide (270 mg) in DMF (1 mL) was added. The resulting mixture was stirred for 15 minutes at room temperature. LC/MS showed only starting material. The reaction mixture was stirred at 150° C. for 30 minutes. The reaction was filtered and purified on reverse phase HPLC (HCl modifier, 25-7₅% ACN-H₂O) to give N-(4-tert-butyl-6-phenoxy-pyrimidin-2-yl)benzenesulfonamide (47.5 mg)¹H NMR (400 MHz, DMSO) δ 11.50 (s, 1H), 7.64-7.47 (m, 5H), 7.41 (s, 2H), 7.35 (t, J=7.4 Hz, 1H), 7.23-7.15 (m, 2H), 6.56 (s, 1H), 1.15 (s, 9H). ESI-MS m/z calc. 383.13037, found 384.0 (M+1)⁺; Retention time: 1.87 minutes; LC method A.

### Example 119: Preparation of Compound 386

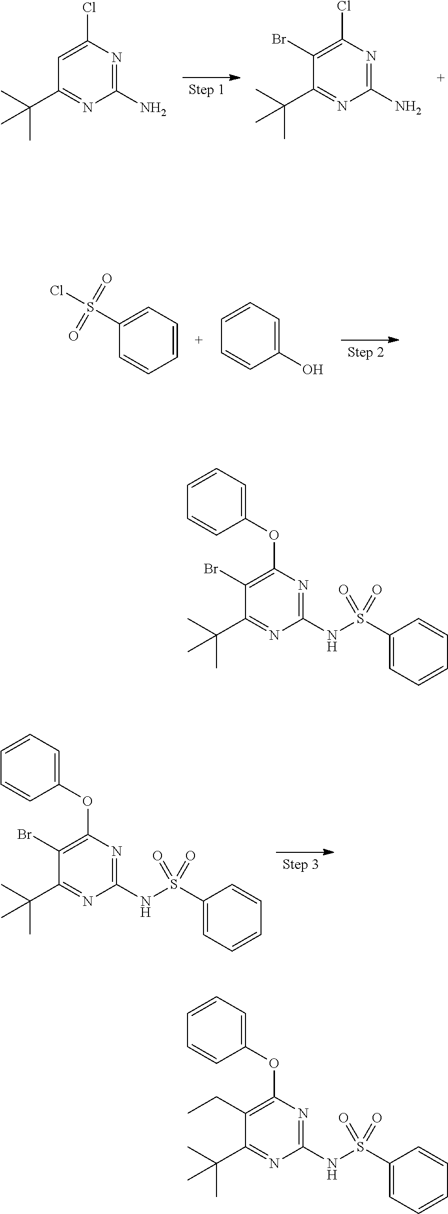

### Step 1: 5-Bromo-4-tert-butyl-6-chloro-pyrimidin-2-amine

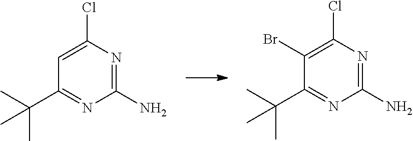

Molecular bromine (approximately 1.377 g, 443.9 μL, 8.618 mmol) was added dropwise to 4-tert-butyl-6-chloro-pyrimidin-2-amine (1.6 g, 8.618 mmol) in acetic acid (30 mL) and the mixture was stirred overnight at room temperature. The reaction mixture was cooled down with ice and the reaction was quenched with ice chips. The precipitate was filtered, washed with water to give 1.31 g of ivory-colored solid. The filtrate was extracted with ethyl acetate (2×20 mL). The organic layer was dried over Na₂SO₄, concentrated and purified on silica using a gradient of ethyl acetate/hexane. The product came out ˜ 15% ethyl acetate to give another 0.276 g of product. Total amount: 5-bromo-4-tert-butyl-6-chloro-pyrimidin-2-amine (1.58 g, 68%)¹H NMR (400 MHz, DMSO) δ 7.15 (s, 2H), 1.41 (s, 9H). ESI-MS m/z calc. 262.98248, found 264.0 (M+1)⁺; Retention time: 1.7 minutes; LC method A.

### Step 2: N-(5-bromo-4-tert-butyl-6-phenoxy-pyrimidin-2-yl)benzenesulfonamide

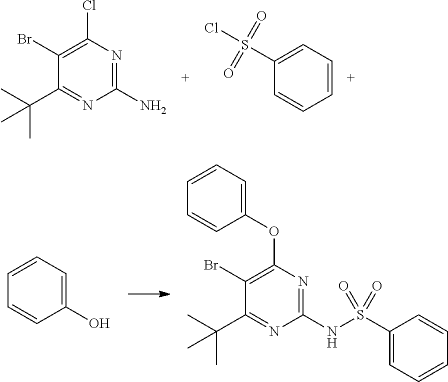 [SMILES: CC(C)(C)c1nc(N)nc(Cl)c1Br ; O=S(=O)(Cl)c1ccccc1 ; Oc1ccccc1 ; CC(C)(C)c1nc(NS(=O)(=O)c2ccccc2)nc(Oc2ccccc2)c1Br]

To a solution of 5-bromo-4-tert-butyl-6-chloro-pyrimidin-2-amine (0.7 g, 2.620 mmol) in DMF (10 mL) was added sodium hydride (approximately 136.2 mg of 60% w/w, 3.406 mmol) at 0° C. and stirred for 10 minutes at 0° C. To this mixture benzenesulfonyl chloride (approximately 555.3 mg, 401.2 μL, 3.144 mmol) was added dropwise and was stirred at 0° C. for 15 minutes. UPLC showed the formation of product with some starting material remained. The reaction mixture was quenched with water and acidified with 1 N HCl, extracted with ethyl aceate (2×10 mL). The organic layer was separated, dried over Na₂SO₄, concentrated and the residue was purified by silica. Product came out ˜ 20% ethyl acetate. 165 mg of starting material was recovered. N-(5-bromo-4-tert-butyl-6-chloro-pyrimidin-2-yl)benzenesulfonamide (437 mg)¹H NMR (400 MHz, DMSO) δ 12.25 (s, 1H), 7.98-7.90 (m, 2H), 7.71-7.65 (m, 1H), 7.65-7.57 (m, 2H), 1.29 (s, 9H). ESI-MS m/z calc. 402.97568, found 405.0 (M+1)⁺; Retention time: 1.95 minutes; LC method A.

NaH (approximately 104.8 mg of 60% w/w, 2.620 mmol) was added to phenol (approximately 246.6 mg, 232.6 μL, 2.620 mmol) in DMF (1 mL) at rt. The mixture was stirred for 10 minutes. and N-(5-bromo-4-tert-butyl-6-chloro-pyrimidin-2-yl)benzenesulfonamide (437 mg) in was added. The resulting mixture was stirred for 15 minutes at room temperature. LC/MS showed only starting material. The reaction mixture was stirred at 150° C. for 30 minutes. LC/MS showed 95% conversion. The reaction was filtered and purified on reverse phase HPLC (HCl modifier, 25-75% ACN-H₂O) to give N-(5-bromo-4-tert-butyl-6-phenoxy-pyrimidin-2-yl)benzenesulfonamide (357 mg) ¹H NMR (400 MHz, CDCl₃) δ 7.56-7.50 (m, 4H), 7.47 (ddt, J=7.8, 7.2, 1.3 Hz, 1H), 7.40-7.33 (m, 1H), 7.30-7.23 (m, 2H), 7.20-7.14 (m, 2H), 1.44 (s, 9H). ESI-MS m/z calc. 461.04086, found 464.0 (M+1)⁺; Retention time: 2.15 minutes; LC method A.

### Step 3: N-(4-tert-butyl-6-phenoxy-5-vinyl-pyrimidin-2-yl)benzenesulfonamide (Compound 386) and N-(4-tert-butyl-5-ethyl-6-phenoxy-pyrimidin-2-yl)benzenesulfonamide

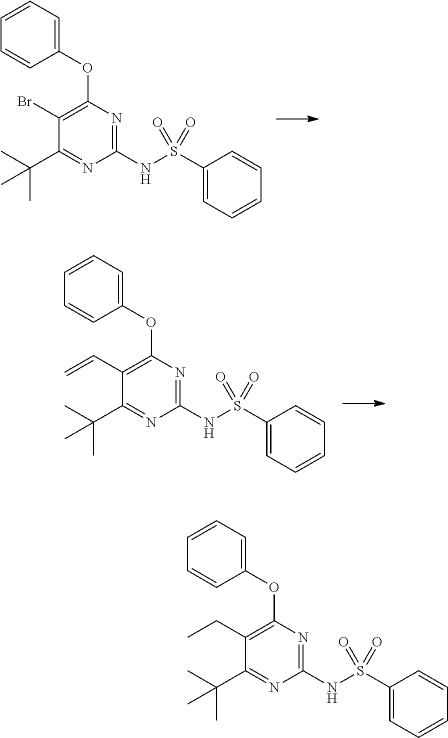 [SMILES: CC(C)(C)c1nc(NS(=O)(=O)c2ccccc2)nc(Oc2ccccc2)c1Br ; C=Cc1c(Oc2ccccc2)nc(NS(=O)(=O)c2ccccc2)nc1C(C)(C)C ; CCc1c(Oc2ccccc2)nc(NS(=O)(=O)c2ccccc2)nc1C(C)(C)C]

To a mixture of N-(5-bromo-4-tert-butyl-6-phenoxy-pyrimidin-2-yl)benzenesulfonamide (60 mg, 0.1298 mmol), 4,4,5,5-tetramethyl-2-vinyl-1,3,2-dioxaborolane (40 mg, 0.2597 mmol), in DMF (1 mL) was added, [1,1′-Bis(diphenylphosphino)ferrocene]dichloropalladium(II), complex with dichloromethane, sodium carbonate (approximately 259.6 μL of 2 M, 0.5192 mmol). The mixture was thoroughly flushed with nitrogen and heated at 100° C. for 1 hour. The reaction mixture was filtered and purified by reverse phase HPLC using 30-99% acetonitrile in water using HCl as modifier: N-(4-tert-butyl-6-phenoxy-5-vinyl-pyrimidin-2-yl)benzenesulfonamide (20 mg)¹H NMR (400 MHz, DMSO) δ 11.46 (s, 1H), 7.59-7.47 (m, 5H), 7.40 (t, J=7.8 Hz, 2H), 7.32 (t, J=6.9 Hz, 1H), 7.14 (d, J=8.6 Hz, 2H), 6.85-6.73 (m, 1H), 5.58 (d, J=7.5 Hz, 1H), 5.55 (s, 1H), 1.25 (s, 9H). ESI-MS m/z calc. 409.14603, found 410.0 (M+1)⁺; Retention time: 2.04 minutes; LC method A.

Pd (28 mg of 5% w/w, 0.01316 mmol) was added to N-(4-tert-butyl-6-phenoxy-5-vinyl-pyrimidin-2-yl)benzenesulfonamide (20 mg) in methanol (5 μL). The reaction mixture was purged with nitrogen and stirred at room temperature for 3 hours. The mixture was filtered, concentrated, and purified on reverse phase HPLC (HCl modifier, 30-99% ACN-H₂O) to give N-(4-tert-butyl-5-ethyl-6-phenoxy-pyrimidin-2-yl)benzenesulfonamide (11.3 mg)¹H NMR (400 MHz, DMSO) δ 11.28 (s, 1H), 7.58-7.52 (m, 2H), 7.51-7.48 (m, 3H), 7.39 (t, J=7.8 Hz, 2H), 7.36-7.30 (m, 1H), 7.18-7.12 (m, 2H), 2.77 (q, J=7.2 Hz, 2H), 1.27 (s, 9H), 1.17 (t, J=7.3 Hz, 3H). ESI-MS m/z calc. 411.16165, found 412.0 (M+1)⁺; Retention time: 2.08 minutes; LC method A.

### Example 120: Preparation of Compound 387

### Step 1: N-(4-chloro-5,6-dimethyl-pyrimidin-2-yl)benzenesulfonamide

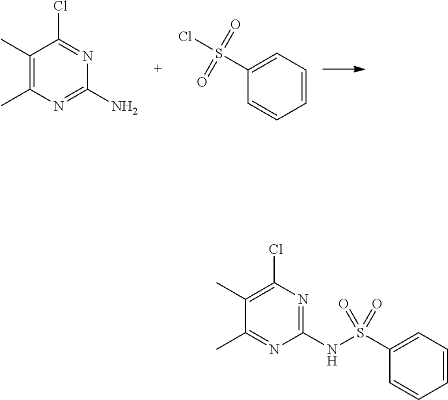

To a solution of 4-chloro-5,6-dimethyl-pyrimidin-2-amine (400 mg, 2.538 mmol) in DMA (3.5 mL) was added NaH (102 mg of 60% w/w, 2.550 mmol). The reaction was stirred at room temperature for 15 minutes. benzenesulfonyl chloride (325 μL, 2.547 mmol) was added and the reaction was stirred at room temperature for 15 hours. The reaction was quenched with MeOH and the solvent was evaporated under reduced pressure. EtOAc was added to the reaction and washed with water (×3). The organic layer was dried over Na₂SO₄, filtered and concentrated. The crude product was purified on 80 g of silica gel utilizing a gradient of 0-50% ethyl acetate in hexane to yield N-(4-chloro-5,6-dimethyl-pyrimidin-2-yl)benzenesulfonamide (114 mg, 15%) as a white solid. ESI-MS m/z calc. 297.03387, found 298.1 (M+1)⁺; Retention time: 1.33 minutes; LC method A.

### Step 2: N-(4,5-Dimethyl-6-phenoxy-pyrimidin-2-yl)benzenesulfonamide

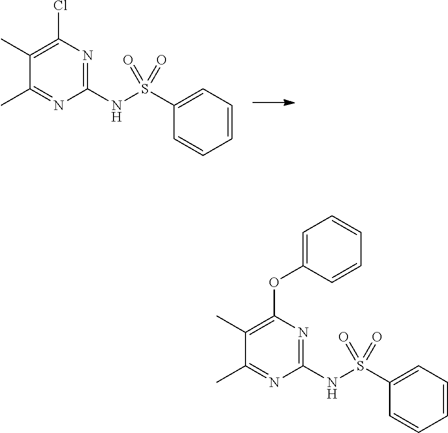 [SMILES: Cc1nc(NS(=O)(=O)c2ccccc2)nc(Cl)c1C ; Cc1nc(NS(=O)(=O)c2ccccc2)nc(Oc2ccccc2)c1C]

To N-(4-chloro-5,6-dimethyl-pyrimidin-2-yl)benzenesulfonamide (62 mg, 0.2082 mmol), sodium phenoxide (129 mg, 1.111 mmol) and N,N-dimethyl formamide (1.5 mL) were added and the reaction was stirred at 110° C. for 2 hours and 15 minutes in a pressure vessel. The crude product was filtered and purified using a reverse phase HPLC C₁₈ column and a dual gradient run from 1-99% mobile phase B over 30 minutes (Mobile phase A=H₂O (5 mM HCl). Mobile phase B═CH₃CN) to yield N-(4,5-dimethyl-6-phenoxy-pyrimidin-2-yl)benzenesulfonamide (18.6 mg, 25%) as a white solid. ¹H NMR (400 MHz, DMSO-d₆) δ 7.61-7.50 (m, 2H), 7.49-7.35 (m, 2H), 7.34-7.17 (m, 4H), 7.17-7.12 (m, 2H), 2.30 (s, 3H), 2.06 (s, 3H). ESI-MS m/z calc. 355.09906, found 356.3 (M+1)⁺; Retention time: 1.39 minutes; LC method A.

### Example 121: Preparation of Compound 388

### Step 1: N-(4-tert-butyl-5-isopropenyl-6-phenoxy-pyrimidin-2-yl)benzenesulfonamide

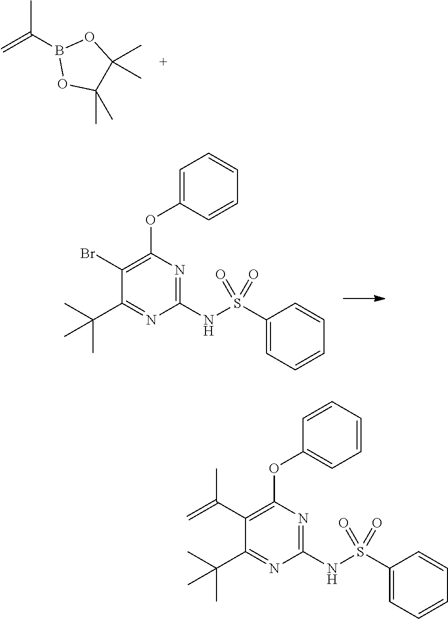 [SMILES: C=C(C)B1OC(C)(C)C(C)(C)O1 ; CC(C)(C)c1nc(NS(=O)(=O)c2ccccc2)nc(Oc2ccccc2)c1Br ; C=C(C)c1c(Oc2ccccc2)nc(NS(=O)(=O)c2ccccc2)nc1C(C)(C)C]

To a mixture of N-(5-bromo-4-tert-butyl-6-phenoxy-pyrimidin-2-yl)benzenesulfonamide (25 mg, 0.05407 mmol), 2-isopropenyl-4,4,5,5-tetramethyl-1,3,2-dioxaborolane (approximately 18.17 mg, 0.1081 mmol), in DMF (1 mL) was added, [1,1′-Bis(diphenylphosphino)ferrocene]dichloropalladium(II), complex with dichloromethane, sodium carbonate (approximately 108.2 μL of 2 M, 0.2163 mmol). The mixture was thoroughly flushed with nitrogen and heated at 100° C. for 1 hour. The reaction mixture was filtered and purified by reverse phase HPLC using 30-99% acetonitrile in water using HCl as modifier to give N-(4-tert-butyl-5-isopropenyl-6-phenoxy-pyrimidin-2-yl)benzenesulfonamide (10 mg, 44%). ESI-MS m/z calc. 423.16165, found 424.0 (M+1)⁺; Retention time: 2.12 minutes; LC method A.

### Example 122: Preparation of Compound 389

### Step 1: N-[4-(cyclohexoxy)-6-cyclohexyl-pyrimidin-2-yl]-1-methyl-pyrazole-4-sulfonamide

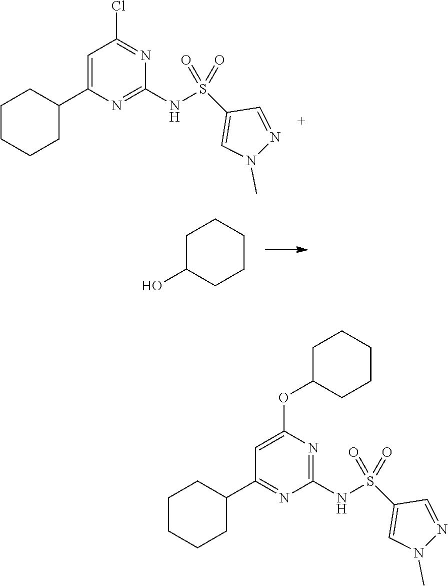

N-(4-chloro-6-cyclohexyl-pyrimidin-2-yl)-1-methyl-pyrazole-4-sulfonamide (30 mg, 0.08431 mmol), Cs₂CO₃ (approximately 137.4 mg, 0.4216 mmol), and cyclohexanol (approximately 33.77 mg, 35.10 μL, 0.3372 mmol) were combined in anhydrous NMP (0.4 mL). The reaction was heated to 180° C. Upon completion, the reaction mixture was filtered, diluted to 0.8 mL total volume with methanol and purified by Prep HPLC (0-99% MeCN), to give the indicated ether products N-[4-(cyclohexoxy)-6-cyclohexyl-pyrimidin-2-yl]-1-methyl-pyrazole-4-sulfonamide (7 mg, 20%). ESI-MS m/z calc. 419.1991, found 420.3 (M+1)⁺; Retention time: 1.68 minutes; LC method A.

### Example 123: Preparation of Compound 390

### Step 1: N-[4-(Cyclohexoxy)-6-cyclopentyl-pyrimidin-2-yl]-1-methyl-pyrazole-4-sulfonamide

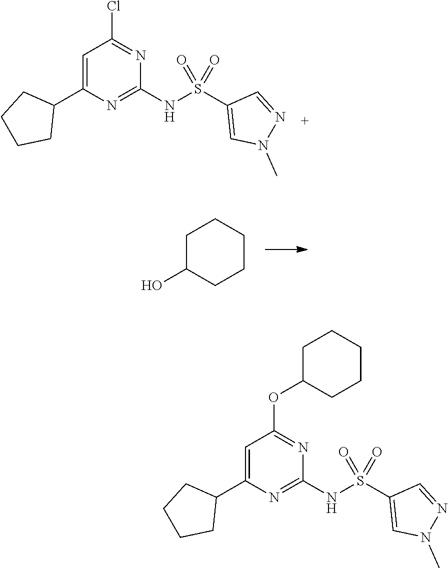 [SMILES: Cn1cc(S(=O)(=O)Nc2nc(Cl)cc(C3CCCC3)n2)cn1 ; OC1CCCCC1 ; Cn1cc(S(=O)(=O)Nc2nc(OC3CCCCC3)cc(C3CCCC3)n2)cn1]

A solution of N-(4-chloro-6-cyclopentyl-pyrimidin-2-yl)-1-methyl-pyrazole-4-sulfonamide (25 mg, 0.07314 mmol) in NMP (400 μL) was added to cyclohexanol (approximately 29.31 mg, 30.47 μL, 0.2926 mmol). cesium carbonate (120 mg, 0.3683 mmol) was added, and the reaction mixture was allowed to stir at 100° C. overnight. It was then heated to 180° C. for one hour followed by 195° C. for another 3 hours. The reaction mixture was then cooled to room temperature, diluted with DMSO and purified by reverse-phase HPLC: Samples were purified using a reverse phase HPLC method using a Luna C₁₈ (2) column (50×21.2 mm, 5 μm particle size) sold by Phenomenex (pn: 00B-4252-PO-AX), and a dual gradient run from 10-70% mobile phase B over 15.0 minutes. Mobile phase A=water (5 mM HCl acid modifier). Mobile phase B=acetonitrile. Flow rate=35 mL/min, injection volume=950 μL, and column temperature=25° C. The UV trace at 220 nm was used to collect fractions. N-[4-(cyclohexoxy)-6-cyclopentyl-pyrimidin-2-yl]-1-methyl-pyrazole-4-sulfonamide (3.9 mg, 13%). ESI-MS m z calc. 405.18347, found 406.3 (M+1)⁺; Retention time: 1.59 minutes; LC method A.

### Example 124: Preparation of Compound 391

### Step 1: 5-Fluoro-4-phenyl-pyrimidin-2-amine

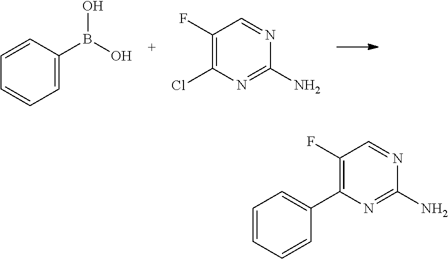 [SMILES: OB(O)c1ccccc1 ; Nc1ncc(F)c(Cl)n1 ; Nc1ncc(F)c(-c2ccccc2)n1]

A mixture of 4-chloro-5-fluoro-pyrimidin-2-amine (200 mg, 1.356 mmol), phenylboronic acid (190 mg, 1.558 mmol), Pd(dppf)C₁₂ (100 mg, 0.1367 mmol), and potassium carbonate (1.5 mL of 2 M, 3.000 mmol) in 1,2-dimethoxyethane (3 mL) was degassed by flow of nitrogen and stirred at 130° C. for 5 hours. The reaction was filtered. EtOAc and water were added to the reaction and the two layers were separated. The organic layer was dried over Na₂SO₄, filtered through a plug of celite and concentrated. The crude product was purified on 40 g of silica gel utilizing a gradient of 0-50% ethyl acetate in hexane to yield a cream solid, 5-fluoro-4-phenyl-pyrimidin-2-amine (164 mg, 64%) ESI-MS m/z calc. 189.07022, found 190.2 (M+1)⁺; Retention time: 1.05 minutes; LC method A.

### Step 2: N-(5-fluoro-4-phenyl-pyrimidin-2-yl)benzenesulfonamide

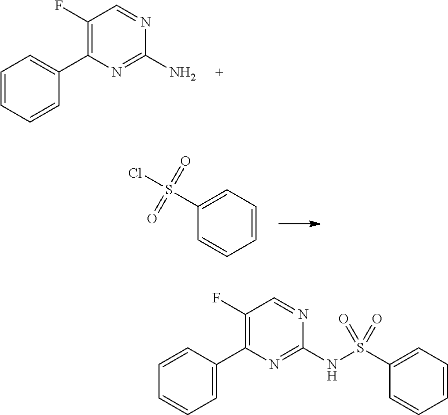

To a solution of 5-fluoro-4-phenyl-pyrimidin-2-amine (85 mg, 0.4493 mmol) in pyridine (1.25 mL) was added benzenesulfonyl chloride (58 μL, 0.4545 mmol) and the reaction was stirred at room temperature for 15 minutes. Very little product was observed by LCMS. The reaction was heated at 200° C. for 10 minutes. More product was observed by UPLC. benzenesulfonyl chloride (58 μL, 0.4545 mmol) was added to the reaction and heated at 200° C. for 2 hours. EtOAc was added to the reaction and washed with water (×3). The organic layer was dried over Na₂SO₄, filtered and concentrated to yield a yellow solid, N-(5-fluoro-4-phenyl-pyrimidin-2-yl)benzenesulfonamide (114 mg, 77%) ESI-MS m/z calc. 329.06342, found 330.1 (M+1)⁺; Retention time: 1.54 minutes; LC method A.

### Step 3: N-(5-phenoxy-4-phenyl-pyrimidin-2-yl)benzenesulfonamide (Compound 391)

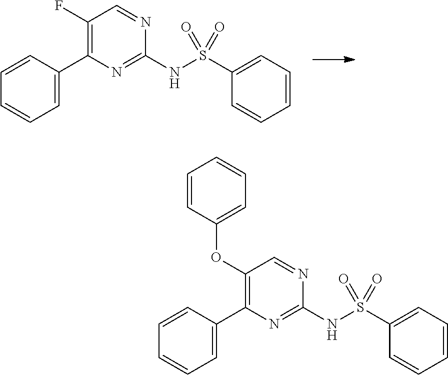 [SMILES: O=S(=O)(Nc1ncc(F)c(-c2ccccc2)n1)c1ccccc1 ; O=S(=O)(Nc1ncc(Oc2ccccc2)c(-c2ccccc2)n1)c1ccccc1]

To N-(5-fluoro-4-phenyl-pyrimidin-2-yl)benzenesulfonamide (113 mg, 0.3431 mmol), sodium phenoxide (81 mg, 0.6977 mmol) and DMF (1.4 mL) were added and the reaction was stirred at room temperature for 0.5 hour in a pressure vessel. Desired product was not observed by UPLC. The reaction was heated at 200° C. for 75 minutes. Very little product was observed by UPLC. Sodium phenoxide (81 mg, 0.6977 mmol), sodium phenoxide (81 mg, 0.6977 mmol) were added to the reaction and heated at 200° C. for 16 hour. sodium phenoxide (81 mg, 0.6977 mmol), sodium phenoxide (81 mg, 0.6977 mmol) were added to the reaction and heated at 200° C. for 6 hour. sodium phenoxide (81 mg, 0.6977 mmol), sodium phenoxide (81mg, 0.6977 mmol) were added to the reaction and heated at 200° C. for 17 hours. Water and EtOAc were added to the reaction and the two layers were separated. The aqueous layer was extracted with EtOAc (×3). The combined organic layer was washed with water (×3), dried over Na₂SO₄, filtered and the solvent was evaporated under reduced pressure. The crude product was purified on 40 g of silica gel utilizing a gradient of 0-30% ethyl acetate in hexane to yield 44 mg product which contained some impurity. The product was dissolved in DMSO, filtered and re-purified using a reverse phase HPLC C₁₈ column and a dual gradient run from 1-99% mobile phase B over 30 minutes [(Mobile phase A=H₂O (5 mM HCl). Mobile phase B═CH₃CN)] to yield a white solid, N-(5-phenoxy-4-phenyl-pyrimidin-2-yl)benzenesulfonamide (31.0 mg, 22%). ¹H NMR (400 MHz, DMSO-d₆) δ 11.96 (s, 1H), 8.41 (s, 1H), 8.05-7.95 (m, 2H), 7.88-7.79 (m, 2H), 7.69-7.63 (m, 1H), 7.63-7.56 (m, 2H), 7.51-7.41 (m, 3H), 7.33-7.26 (m, 2H), 7.10-7.01 (m, 1H), 6.98-6.87 (m, 2H). ESI-MS m/z calc. 403.09906, found 404.2 (M+1)⁺; Retention time: 1.81 minutes; LC method A.

### Example 125: Preparation of Compound 392

### Step 1: N-[5-(2,4-Dimethylphenyl)pyrimidin-2-yl]benzenesulfonamide

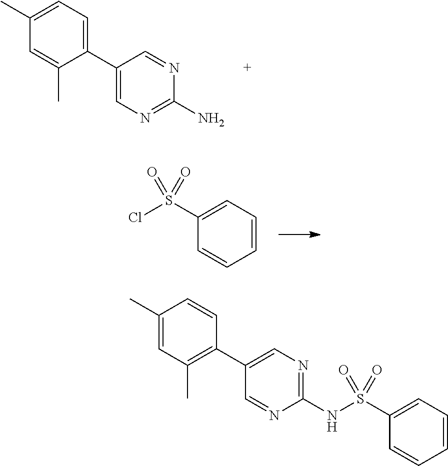 [SMILES: Cc1ccc(-c2cnc(N)nc2)c(C)c1 ; O=S(=O)(Cl)c1ccccc1 ; Cc1ccc(-c2cnc(NS(=O)(=O)c3ccccc3)nc2)c(C)c1]

To a solution of 5-(2,4-dimethylphenyl)pyrimidin-2-amine (19.93 mg, 0.1 mmol) in CH₃CN (0.5 mL) was added DABCO (approximately 56.09 mg, 0.5000 mmol) followed by benzenesulfonyl chloride (approximately 35.32 mg, 25.52 μL, 0.2000 mmol) and the reaction mixture stirred at room temperature for 4 hours. The reaction mixture was diluted with MeOH, filtered and purification by reverse phase HPLC (1-99% ACN in water (HCl modifier)) gave N-[5-(2,4-dimethylphenyl)pyrimidin-2-yl]benzenesulfonamide (4.0 mg, 12%). ESI-MS m/z calc. 339.10416, found 340.3 (M+1)⁺; Retention time: 1.63 minutes; LC method A.

### Example 126: Preparation of Compound 393

### Step 1: 4,5-Diphenylpyrimidin-2-amine

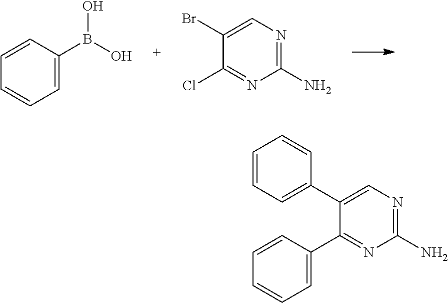 [SMILES: OB(O)c1ccccc1 ; Nc1ncc(Br)c(Cl)n1 ; Nc1ncc(-c2ccccc2)c(-c2ccccc2)n1]

A solution of 5-bromo-4-chloro-pyrimidin-2-amine (152 mg, 0.7292 mmol), 5-bromo-4-chloro-pyrimidin-2-amine (152 mg, 0.7292 mmol), Pd(ddpf)C₁₂ (67 mg, 0.09157 mmol) and sodium carbonate (1.5 mL of 2 M, 3.000 mmol) in DME (1.5 mL) was stirred at 80° C. for 16 hours. The reaction mixture was diluted with water and extracted with EtOAc (2×). Organics were combined and evaporated to dryness. Purification by column chromatography (12 g silica; 0-40% EtOAc in hexanes) gave a white solid, 4,5-diphenylpyrimidin-2-amine (70 mg, 39%). ESI-MS m/z calc. 247.11095, found 248.2 (M+1)⁺; Retention time: 0.47 minutes; LC method D.

### Step 2: N-(4,5-Diphenylpyrimidin-2-yl)benzenesulfonamide (Compound 393)

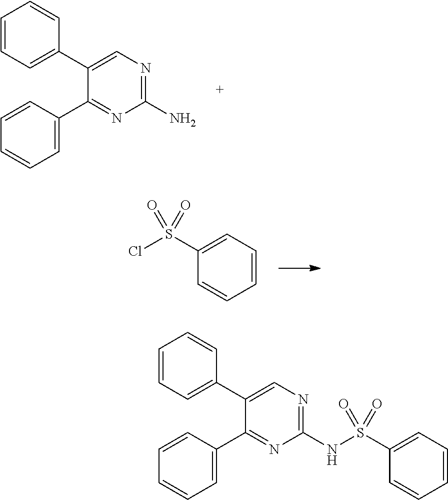 [SMILES: Nc1ncc(-c2ccccc2)c(-c2ccccc2)n1 ; O=S(=O)(Cl)c1ccccc1 ; O=S(=O)(Nc1ncc(-c2ccccc2)c(-c2ccccc2)n1)c1ccccc1]

A mixture of 4,5-diphenylpyrimidin-2-amine (17 mg, 0.06874 mmol) and benzenesulfonyl chloride (250 μL, 1.959 mmol) was heated with a heat gun until the solution was at reflux and held at reflux for 30 seconds then cooled. The solution was subject to 30 seconds at reflux with a heat gun 3 more times. The reaction mixture was diluted with DMSO, filtered and purification by reverse phase HPLC (1-99% ACN in water (HCl modifier)) gave an off-white solid, N-(4,5-diphenylpyrimidin-2-yl)benzenesulfonamide (5.6 mg, 21%). ESI-MS m z calc. 387.10416, found 388.2 (M+1)⁺; Retention time: 1.75 minutes; LC method A.

### Example 127: Preparation of Compound 394

### Step 1: N-(5-bromo-4-chloro-pyrimidin-2-yl)benzenesulfonamide

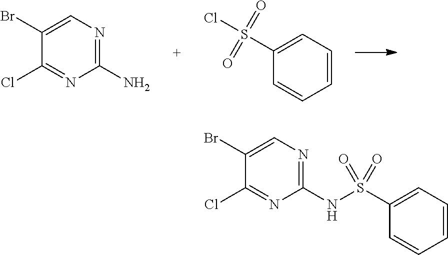

To a solution of 5-bromo-4-chloro-pyrimidin-2-amine (1.22 g, 5.853 mmol) in DMA (7.6 mL) was added NaH (235 mg of 60% w/w, 5.876 mmol). The reaction was stirred at room temperature for 15 minutes. benzenesulfonyl chloride (748 μL, 5.861 mmol) was added and the reaction was stirred at room temperature for 5.5 hours. Benzenesulfonyl chloride (748 μL, 5.861 mmol) was added to the reaction and stirred at room temperature for 16 hours. The reaction was quenched with MeOH and the solvent was evaporated under reduced pressure. EtOAc was added to the reaction and washed with water (×3). The organic layer was dried over Na₂SO₄, filtered and concentrated. The crude product was purified on 120 g of silica gel utilizing a gradient of 0-30% ethyl acetate in hexane to yield a yellow solid, N-(5-bromo-4-chloro-pyrimidin-2-yl)benzenesulfonamide (470 mg, 23%). ESI-MS m/z calc. 346.9131, found 348.0 (M+1)⁺; Retention time: 1.39 minutes; LC method A.

### Step 2: N-(5-bromo-4-phenoxy-pyrimidin-2-yl)benzenesulfonamide

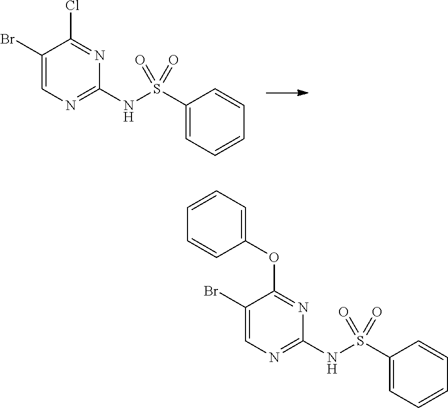

To N-(5-bromo-4-chloro-pyrimidin-2-yl)benzenesulfonamide (450 mg, 1.291 mmol), sodium phenoxide (approximately 630.3 mg, 5.429 mmol) and NN-dimethyl formamide (12 mL) were added and the reaction was stirred at 110° C. for 20 minutes in a pressure vessel. The crude product was purified on 220 g of silica gel utilizing a gradient of 0-30% ethyl acetate in hexane. The solvent was evaporated under reduced pressure. The product was slurried in hexane twice and the solvent was evaporated under reduced pressure to yield a cream solid, N-(5-bromo-4-phenoxy-pyrimidin-2-yl)benzenesulfonamide (290 mg, 55%). ESI-MS m/z calc. 404.97827, found 406.0 (M+1)⁺; Retention time: 1.61 minutes; LC method A.

### Step 3: N-(4-phenoxy-5-phenyl-pyrimidin-2-yl)benzenesulfonamide (Compound 394)

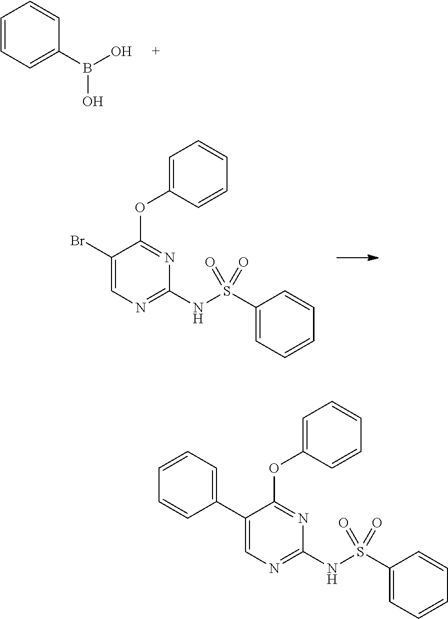 [SMILES: OB(O)c1ccccc1 ; O=S(=O)(Nc1ncc(Br)c(Oc2ccccc2)n1)c1ccccc1 ; O=S(=O)(Nc1ncc(-c2ccccc2)c(Oc2ccccc2)n1)c1ccccc1]

A mixture of N-(5-bromo-4-phenoxy-pyrimidin-2-yl)benzenesulfonamide (88 mg, 0.2166 mmol), phenylboronic acid (48 mg, 0.3937 mmol), Pd(dppf)C₁₂ (21 mg, 0.02870 mmol), 1,4-dioxane (2 mL) and potassium carbonate (218 μL of 2 M, 0.4360 mmol) was degassed by a flow of nitrogen and stirred at 110° C. for 35 minutes. The cooled mixture was filtered and concentrated in vacuo. The crude product was dissolved in DMSO, filtered and purified using a reverse phase HPLC C₁₈ column and a dual gradient run from 1-99% mobile phase B over 15 minutes [(Mobile phase A=H₂O (5 mM HCl). Mobile phase B═CH₃CN)] to yield N-(4-phenoxy-5-phenyl-pyrimidin-2-yl)benzenesulfonamide (25.2 mg, 29%) as a cream solid. ¹H NMR (400 MHz, DMSO-d₆) δ 11.99 (s, 1H), 8.43 (s, 1H), 7.69-7.60 (m, 2H), 7.60-7.49 (m, 3H), 7.49-7.42 (m, 2H), 7.42-7.30 (m, 6H), 7.28-7.18 (m, 2H). ESI-MS m/z calc. 403.09906, found 404.2 (M+1)⁺; Retention time: 1.69 minutes; LC method A.

### Example 128: Preparation of Compound 395

### Step 1: N-[5-(o-tolyl)-4-phenoxy-pyrimidin-2-yl]benzenesulfonamide

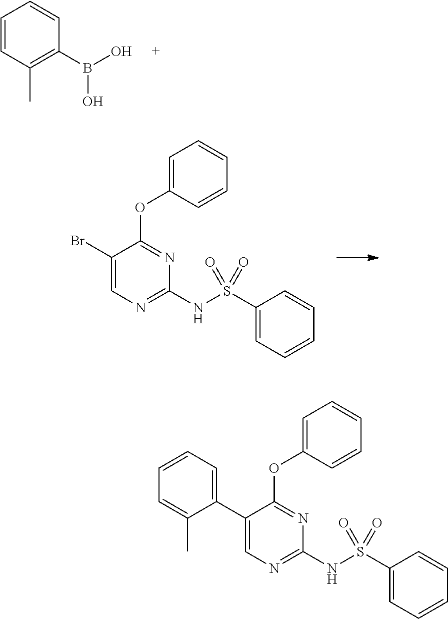

A mixture of N-(5-bromo-4-phenoxy-pyrimidin-2-yl)benzenesulfonamide, methylboronic acid, Pd(dppf)C₁₂ (13.50 mg, 0.01845 mmol), 1,4-dioxane (500.0 μL) and potassium carbonate (62 μL of 2 M, 0.1240 mmol) was degassed by flow of nitrogen and stirred at 110° C. for 1-2 hours. The mixture was cooled down to room temperature, filtered and concentrated in vacuo. The crude product was dissolved in DMSO, filtered and purified using a reverse phase HPLC C₁₈ column and a dual gradient run from 1-99% mobile phase B over 30 minutes (Mobile phase A=H₂O(5 mM HCl). Mobile phase B═CH₃CN) to yield N-[5-(o-tolyl)-4-phenoxy-pyrimidin-2-yl]benzenesulfonamide (9.4 mg, 37%). ESI-MS m/z calc. 417.11472, found 418.2 (M+1)⁺; Retention time: 1.74 minutes; LC method A.

### Example 129: Preparation of Compound 396

### Step 1: N-(5-bromo-4-chloro-pyrimidin-2-yl)-3-nitro-benzenesulfonamide

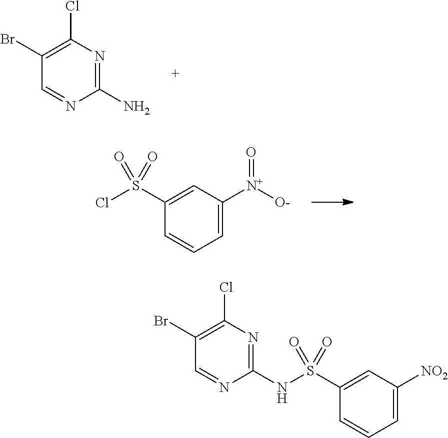

To a solution of 5-bromo-4-chloro-pyrimidin-2-amine (2 g, 9.595 mmol) in DMF (38 mL) at 0° C. was added sodium hydride (1.54 g of 60% w/w, 38.50 mmol). The reaction was allowed to warm to 23° C. over 15 minutes. The reaction was cooled back to 0° C. and 3-nitrobenzenesulfonyl chloride (4.25 g, 19.18 mmol) was introduced in one portion. Stirring continued for 15 minutes before acidifying with acetic acid (8.64 g, 143.9 mmol) and diluting with water and ethyl acetate/hexanes (1:1). The organic layer was separated, and the aqueous layer was further extracted with ethyl acetate/hexanes (1:1, 4×). The combined organics were washed twice with brine, dried over magnesium sulfate, filtered, and concentrated onto silica gel. The silica gel was subjected to flash column chromatography (ethyl acetate in hexanes) to afford a yellow solid, N-(5-bromo-4-chloro-pyrimidin-2-yl)-3-nitro-benzenesulfonamide (2.05 g, 52%). ESI-MS m/z calc. 391.89816, found 395.14 (M+3)*; Retention time: 0.6 minutes; LC method D.

### Step 2: N-(5-bromo-4-phenoxy-pyrimidin-2-yl)-3-nitro-benzenesulfonamide

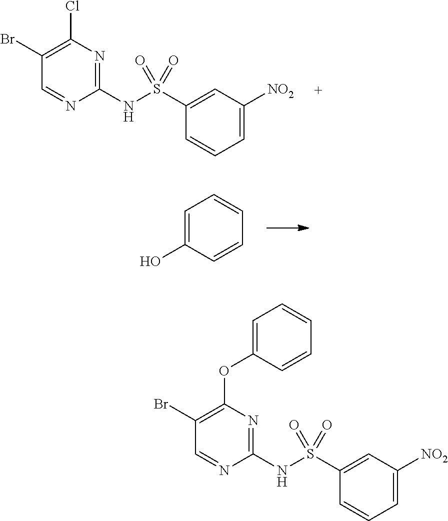

A heterogeneous solution consisting of N-(5-bromo-4-chloro-pyrimidin-2-yl)-3-nitro-benzenesulfonamide (2 g, 5 mmol), potassium carbonate (3 g, 21.71 mmol), and Phenol (600 mg, 6.375 mmol) in NMP (20 mL) was heated to 120° C. for 16 hours. The reaction was cooled and acetic acid (2 g, 33.30 mmol) was added followed by the addition of water and ethyl acetate/hexanes (1:1). The organic layer was separated and the aqueous layer was further extracted with ethyl acetate/hexanes (1:1, 4×). The combined organics were washed with brine (2×), dried over magnesium sulfate, filtered, and concentrated in vacuo. The crude residue was subjected to flash column chromatography on silica gel (eluent: ethyl acetate in hexanes) to afford N-(5-bromo-4-phenoxy-pyrimidin-2-yl)-3-nitro-benzenesulfonamide (1.24 g, 44%) as a yellow solid (purity by UV−VIS=71%). ESI-MS m/z calc. 449.96335, found 451.27 (M+1)⁺; Retention time: 0.64 minutes; LC method D.

### Step 3: 3-Amino-N-(5-bromo-4-phenoxy-pyrimidin-2-yl)benzenesulfonamide

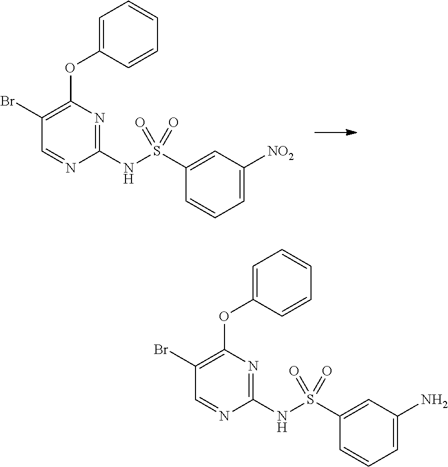 [SMILES: O=[N+]([O-])c1cccc(S(=O)(=O)Nc2ncc(Br)c(Oc3ccccc3)n2)c1 ; Nc1cccc(S(=O)(=O)Nc2ncc(Br)c(Oc3ccccc3)n2)c1]

To a solution of N-(5-bromo-4-phenoxy-pyrimidin-2-yl)-3-nitro-benzenesulfonamide (1 g, 2 mmol) in ethanol (8 mL), DMF (12.6 mL), and acetic acid(5.6 mL) was added Hydrochloric acid (500 μL of 37% w/v, 5.074 mmol) and Iron (600 mg, 10.74 mmol). The reaction was heated to 70° C. for 2 hours then concentrated in vacuo on silica gel. The crude impregnated silica gel was separated by flash column chromatography (ethyl acetate in hexanes) to afford a white solid, 3-amino-N-(5-bromo-4-phenoxy-pyrimidin-2-yl)benzenesulfonamide (910 mg, 97%). ESI-MS m/z calc. 419.98917, found 421.26 (M+1)⁺; Retention time: 0.57 minutes; LC method D.

### Step 4: 3-Amino-N-[5-(2-isopropylphenyl)-4-phenoxy-pyrimidin-2-yl]benzenesulfonamide (Compound 396)

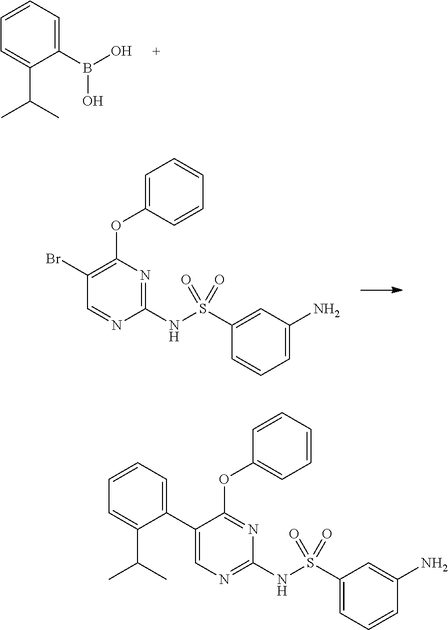

A heterogeneous solution consisting of (2-isopropylphenyl)boronic acid (approximately 29.19 mg, 0.1780 mmol), 3-amino-N-(5-bromo-4-phenoxy-pyrimidin-2-yl)benzenesulfonamide (25 mg, 0.05934 mmol), potassium carbonate (approximately 41.01 mg, 0.2967 mmol), and tetrakis(triphenylphosphine)palladium(0) (approximately 13.72 mg, 0.01187 mmol) in dioxane (247.2 μL) and water (49.45 μL) was microwaved in a sealed vial to 120° C. for 30 minutes. The crude reaction was acidified with acetic acid (approximately 53.45 mg, 50.62 μL, 0.8901 mmol) and diluted with DMSO (0.5 mL). The crude mixture was separated by HPLC (acetonitrile in water with 0.1% hydrochloric acid) to afford the title compound 3-amino-N-[5-(2-isopropylphenyl)-4-phenoxy-pyrimidin-2-yl]benzenesulfonamide (1.9 mg, 7%). ESI-MS m/z calc. 460.15692, found 461.3 (M+1)⁺; Retention time: 1.59 minutes; LC method A.

### Example 130: Preparation of Compound 397

### Step 1: N-(5-isopropenyl-4-phenoxy-pyrimidin-2-yl)benzenesulfonamide

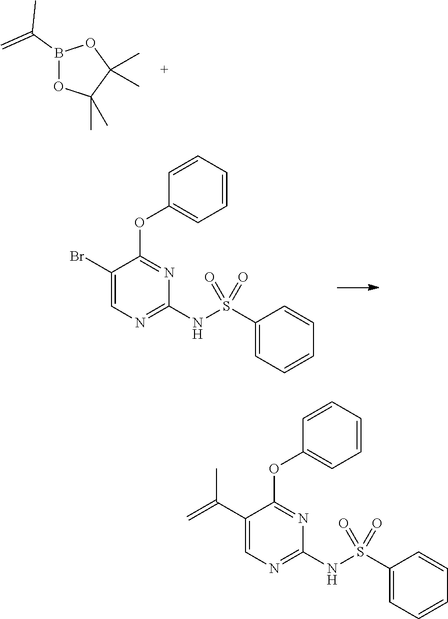 [SMILES: C=C(C)B1OC(C)(C)C(C)(C)O1 ; O=S(=O)(Nc1ncc(Br)c(Oc2ccccc2)n1)c1ccccc1 ; C=C(C)c1cnc(NS(=O)(=O)c2ccccc2)nc1Oc1ccccc1]

A mixture of N-(5-bromo-4-phenoxy-pyrimidin-2-yl)benzenesulfonamide, methylboronic acid, Pd(dppf)C₁₂ (27.0 mg, 0.03690 mmol), 1,4-dioxane (1.000 mL) and potassium carbonate (123 μL of 2 M, 0.2460 mmol) was degassed by flow of nitrogen and stirred at 110° C. for 1-2 hours. Each mixture was cooled down to room temperature, filtered and concentrated in vacuo. Each crude product was dissolved in DMSO, filtered and purified using a reverse phase HPLC C₁₈ column and a dual gradient run from 1-99% mobile phase B over 30 minutes (Mobile phase A=H₂O (5 mM HCl). Mobile phase B═CH₃CN) to yield N-(5-isopropenyl-4-phenoxy-pyrimidin-2-yl)benzenesulfonamide (9.0 mg, 20%). ESI-MS m/z calc. 367.09906, found 368.2 (M+1)⁺; Retention time: 1.6 minutes; LC method A.

### Example 131: Characterization of Compounds 398-412

The compounds in the following tables were prepared in a manner analogous to that described above using commercially available reagents and intermediates described herein.

| | | LCMS | | | |
|---|---|---|---|---|---|
| | | Room | | | |
| Compound | | Temperature | Calc. | | LCMS |
| number | Structure | (min) | mass | M + 1 | method |
| 398 | 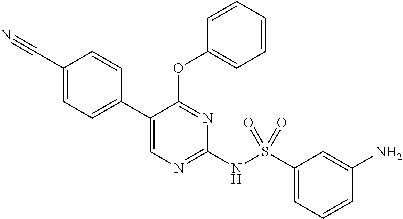 [SMILES: N#Cc1ccc(-c2cnc(NS(=O)(=O)c3cccc(N)c3)nc2Oc2ccccc2)cc1] | 1.38 | 443.105 | 444.25 | A |
| 399 | 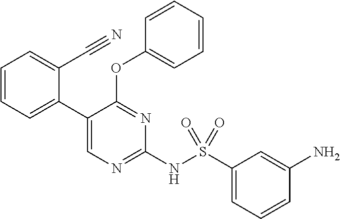 [SMILES: N#Cc1ccccc1-c1cnc(NS(=O)(=O)c2cccc(N)c2)nc1Oc1ccccc1] | 1.49 | 443.105 | 444.44 | A |
| 400 | 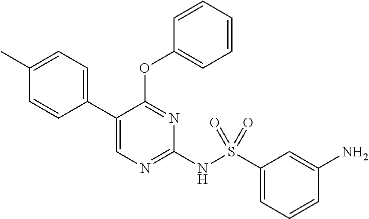 [SMILES: Cc1ccc(-c2cnc(NS(=O)(=O)c3cccc(N)c3)nc2Oc2ccccc2)cc1] | 1.5 | 432.126 | 433.22 | A |
| 401 | 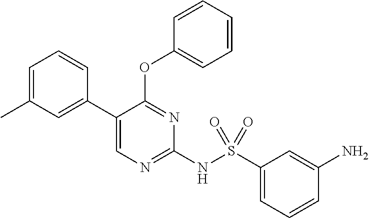 [SMILES: Cc1cccc(-c2cnc(NS(=O)(=O)c3cccc(N)c3)nc2Oc2ccccc2)c1] | 1.5 | 432.126 | 433.22 | A |
| 402 | 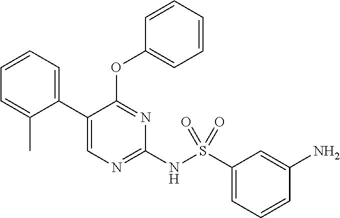 [SMILES: Cc1ccccc1-c1cnc(NS(=O)(=O)c2cccc(N)c2)nc1Oc1ccccc1] | 1.44 | 432.126 | 433.22 | A |
| 403 | 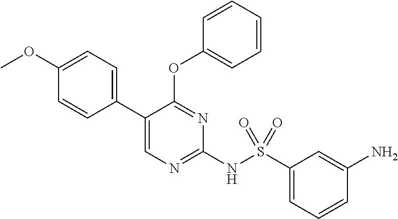 [SMILES: COc1ccc(-c2cnc(NS(=O)(=O)c3cccc(N)c3)nc2Oc2ccccc2)cc1] | 1.38 | 448.121 | 449.28 | A |
| 404 | 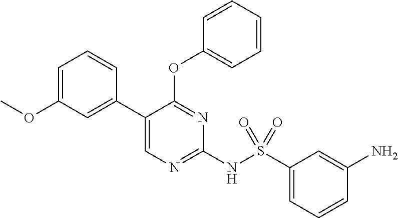 [SMILES: COc1cccc(-c2cnc(NS(=O)(=O)c3cccc(N)c3)nc2Oc2ccccc2)c1] | 1.44 | 448.121 | 449.28 | A |
| 405 | 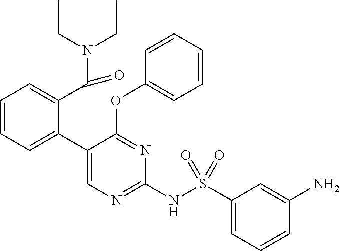 [SMILES: CCN(CC)C(=O)c1ccccc1-c1cnc(NS(=O)(=O)c2cccc(N)c2)nc1Oc1ccccc1] | 1.34 | 517.178 | 518.32 | A |
| 406 | 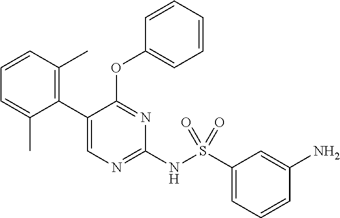 [SMILES: Cc1cccc(C)c1-c1cnc(NS(=O)(=O)c2cccc(N)c2)nc1Oc1ccccc1] | 1.5 | 446.141 | 447.29 | A |
| 407 | 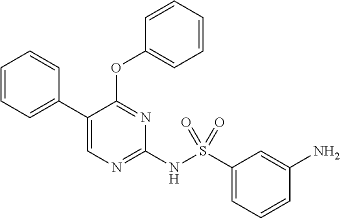 [SMILES: Nc1cccc(S(=O)(=O)Nc2ncc(-c3ccccc3)c(Oc3ccccc3)n2)c1] | 1.39 | 418.11 | 419.21 | A |
| 408 | 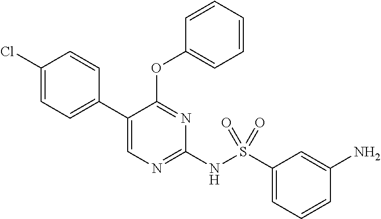 [SMILES: Nc1cccc(S(=O)(=O)Nc2ncc(-c3ccc(Cl)cc3)c(Oc3ccccc3)n2)c1] | 1.56 | 452.071 | 453.17 | A |
| 409 | 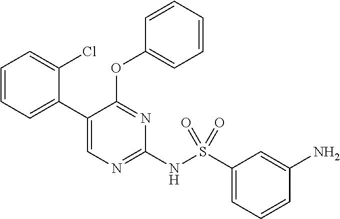 [SMILES: Nc1cccc(S(=O)(=O)Nc2ncc(-c3ccccc3Cl)c(Oc3ccccc3)n2)c1] | 1.46 | 452.071 | 453.17 | A |
| 410 | 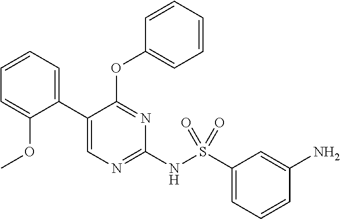 [SMILES: COc1ccccc1-c1cnc(NS(=O)(=O)c2cccc(N)c2)nc1Oc1ccccc1] | 1.4 | 448.121 | 449.09 | A |
| 411 | 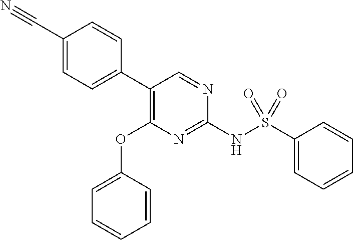 [SMILES: N#Cc1ccc(-c2cnc(NS(=O)(=O)c3ccccc3)nc2Oc2ccccc2)cc1] | 1.6 | 428.094 | 429.2 | A |
| 412 | 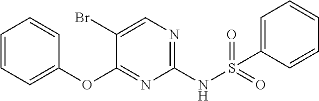 [SMILES: O=S(=O)(Nc1ncc(Br)c(Oc2ccccc2)n1)c1ccccc1] | 1.63 | 404.978 | 406 | A |

| Compound | |
|---|---|
| number | NMR |
| 412 | ¹H NMR (400 MHz, DMSO-d₆) δ 11.93 (s, 1H), 8.60 (d, J = 1.2 Hz, 1H), 7.67 − |
| | 7.51 (m, 3H), 7.47 − 7.39 (m, 1H), 7.38 − 7.31 (m, 4H), 7.28 − 7.16 (m, 2H). |

### Example 132: Preparation of Compound 413

### Step 1: N-(4-tert-butyl-6-chloro-pyrimidin-2-yl)benzenesulfonamide

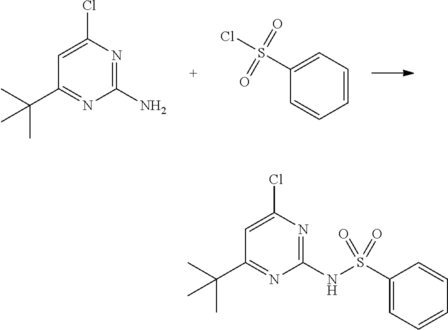 [SMILES: CC(C)(C)c1cc(Cl)nc(N)n1 ; O=S(=O)(Cl)c1ccccc1 ; CC(C)(C)c1cc(Cl)nc(NS(=O)(=O)c2ccccc2)n1]

To a solution of 4-tert-butyl-6-chloro-pyrimidin-2-amine (338.2 mg, 1.822 mmol) in DMF (5 mL) was added sodium hydride (approximately 94.75 mg of 60% w/w, 2.369 mmol) at 0° C. and stirred for 10 minutes at 0° C. To this mixture benzenesulfonyl chloride (approximately 386.1 mg, 279.0 μL, 2.186 mmol) was added dropwise and was stirred at 0° C. for 15 minutes. The reaction mixture was quench with water and acidified with 1 N HCl, extracted with ethyl aceate (2×10 mL). The organic layer was separated, dried over Na₂SO₄, concentrated and the residue was purified by silica. Product came out ˜ 20% ethyl acetate. N-(4-tert-butyl-6-chloro-pyrimidin-2-yl)benzenesulfonamide (270 mg). ¹H NMR (400 MHz, DMSO) δ 12.09 (s, 1H), 7.96 (d, J=8.5 Hz, 2H), 7.65 (d, J=5.8 Hz, 1H), 7.60 (t, J=7.3 Hz, 2H), 7.17 (s, 1H), 1.10 (s, 9H). ESI-MS m/z calc. 325.0652, found 326.0 (M+1)⁺; Retention time: 1.71 minutes; LC method A.

### Example 133: Preparation of Compound 414

### Step 1: N-methyl-N-(4-phenoxy-6-phenyl-pyrimidin-2-yl)benzenesulfonamide

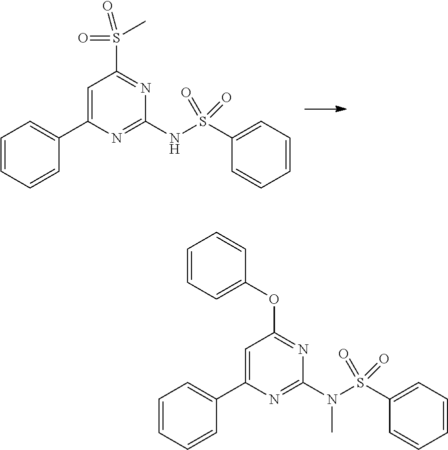 [SMILES: CS(=O)(=O)c1cc(-c2ccccc2)nc(NS(=O)(=O)c2ccccc2)n1 ; CN(c1nc(Oc2ccccc2)cc(-c2ccccc2)n1)S(=O)(=O)c1ccccc1]

To a solution of N-(4-methylsulfonyl-6-phenyl-pyrimidin-2-yl)benzenesulfonamide (38.94 mg, 0.1 mmol) at 0° C. was added NaH (approximately 4.800 mg, 0.1200 mmol) and stirred at this temperature for 5 minutes before the addition of Mel (approximately 17.03 mg, 7.469 μL, 0.1200 mmol). The cooling bath was removed and stirred at room temperature for 15 minutes. At this time more Mel (approximately 17.03 mg, 7.469 μL, 0.1200 mmol) and NaH (approximately 4.800 mg, 0.1200 mmol) was added and stirred at room temperature for 10 minutes. To the reaction mixture was added sodium phenoxide (approximately 58.05 mg, 0.5000 mmol) and the reaction mixture stirred at 100° C. for 20 minutes. The reaction mixture was diluted with MeOH, filtered and purification by HPLC (1-99% ACN in water (HCl modifier)) gave a white solid, N-methyl-N-(4-phenoxy-6-phenyl-pyrimidin-2-yl)benzenesulfonamide (12.5 mg, 30%). ¹H NMR (400 MHz, DMSO-d₆) δ 8.08-8.01 (m, 2H), 7.64-7.49 (m, 8H), 7.46-7.35 (m, 3H), 7.33 (d, J=0.7 Hz, 1H), 7.26 (d, J=8.3, 1.2 Hz, 2H), 3.56 (s, 3H). ESI-MS m/z calc. 417.11472, found 418.3 (M+1)⁺; Retention time: 2.15 minutes; LC method A.

### Example 134: Preparation of Compound 415

### Step 1: N-[4-(2,6-Dimethylphenyl)-6-(2-fluorophenoxy)pyrimidin-2-yl]-1-methyl-pyrazole-4-sulfonamide

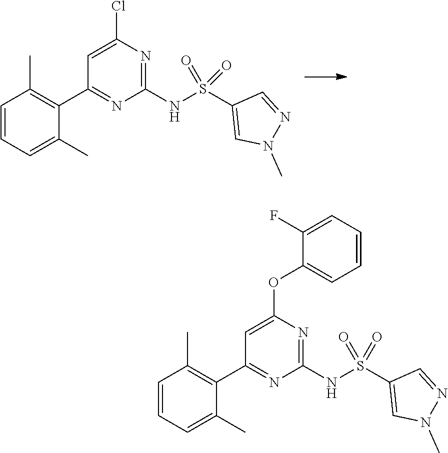

Into a 20 mL glass vial was added N-[4-chloro-6-(2,6-dimethylphenyl)pyrimidin-2-yl]-1-methyl-1H-pyrazole-4-sulfonamide (227 mg, 0.6 mmol), 2-fluorophenol (0.065 mL, 0.72 mmol) and potassium carbonate (166 mg, 1.2 mmol) in acetonitrile (6 mL). The reaction mixture was heated at 80° C. for 16 hours. The reaction was cooled to ambient temperature, and aqueous hydrochloric acid (a N, 10 mL) was added. The reaction solution was extracted with ethyl acetate (3×20 mL). The combined organic layers were washed with brine (20 mL), dried over anhydrous magnesium sulfate and concentrated under vacuum. The residue was purified by silica gel chromatography using 0 to 50% hexane-ethyl acetate to furnish a white solid, N-[4-(2,6-dimethylphenyl)-6-(2-fluorophenoxy)pyrimidin-2-yl]-1-methyl-1H-pyrazole-4-sulfonamide (250 mg, 92%). ¹H NMR (500 MHz, DMSO-d₆) δ (ppm): 11.65 (s, 1H); 7.66 (s, 1H); 7.40 (m, 4H); 7.20 (m, 4H); 6.75 (s, 1H); 3.75 (s, 3H); 2.07 (s, 6H). ESI-MS m/z calc. 453.12708, found 454.0 (M+1)⁺; Retention time: 5.0 minutes.

### Step 2: N-[4-(2,6-dimethylphenyl)-6-(2-fluorophenoxy)pyrimidin-2-yl]-N,1-dimethyl-pyrazole-4-sulfonamide

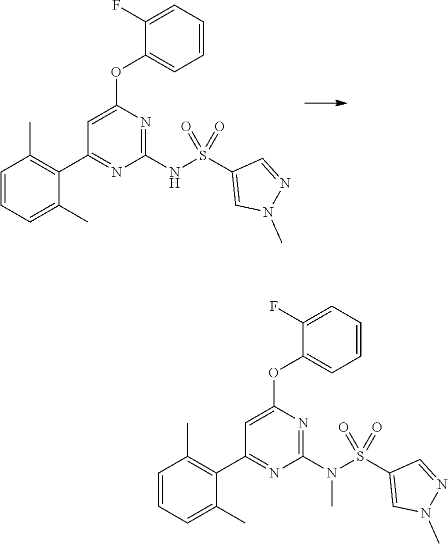 [SMILES: Cc1cccc(C)c1-c1cc(Oc2ccccc2F)nc(NS(=O)(=O)c2cnn(C)c2)n1 ; Cc1cccc(C)c1-c1cc(Oc2ccccc2F)nc(N(C)S(=O)(=O)c2cnn(C)c2)n1]

Into a 50-mL round bottom flask was charged with N-[4-(2,6-dimethylphenyl)-6-(2-fluorophenoxy)pyrimidin-2-yl]-1-methyl-1H-pyrazole-4-sulfonamide (171 mg, 0.377 mmol) in acetonitrile (10 mL). Potassium carbonate (104 mg, 0.754 mmol) was added, and the mixture was stirred at room temperature for 5 minutes. Iodomethane (0.0235 mL, 0.377 mmol) was added to the reaction, and the reaction mixture was stirred at room temperature for 2 days. The solvent was removed under vacuum. The residue was purified by silica gel chromatography using 0 to 50% hexanes-ethyl acetate. The crude product was triturated with 20% diethyl ether in hexane to furnish a white solid, N-[4-(2,6-dimethylphenyl)-6-(2-fluorophenoxy)pyrimidin-2-yl]-N,1-dimethyl-1H-pyrazole-4-sulfonamide (86 mg, 49%). ¹H NMR (250 MHz, CDCl₃) δ (ppm): 7.52 (s, 1H); 7.27 (m, 7H); 7.15 (d, J=7.5 Hz, 1H); 6.51 (s, 1H); 3.73 (s, 3H); 3.44 (s, 3H); 2.13 (s, 6H). ESI-MS m/z calc. 467.14273, found 468.1 (M+1)⁺; Retention time: 6.22 minutes.

### Example 135: Preparation of Compound 416

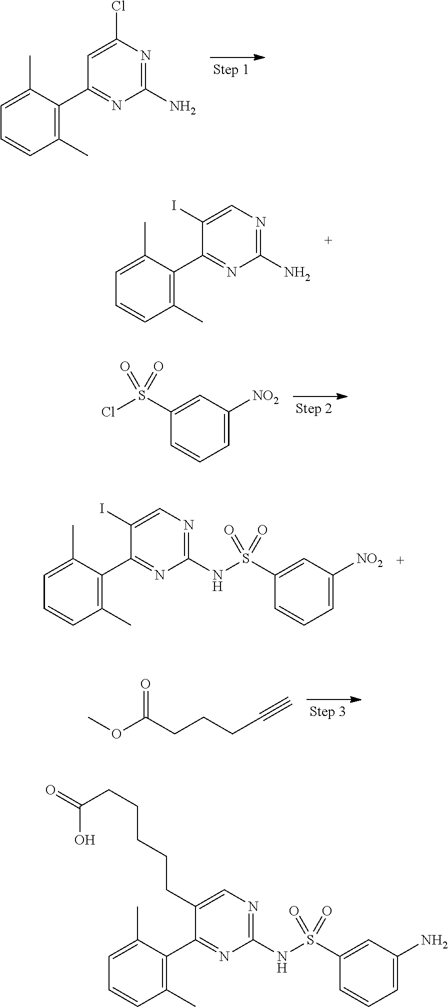

### Step 1: 4-(2,6-Dimethylphenyl)-5-iodo-pyrimidin-2-amine

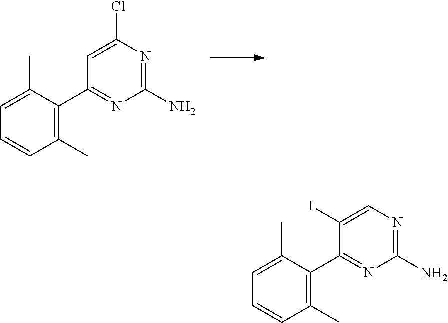 [SMILES: Cc1cccc(C)c1-c1cc(Cl)nc(N)n1 ; Cc1cccc(C)c1-c1nc(N)ncc1I]

Stage 1: To a solution of 4-chloro-6-(2,6-dimethylphenyl)pyrimidin-2-amine (3.1 g, 13.27 mmol) in Ethanol (26 mL) was added Palladium on Carbon (282 mg of 5% w/w, 0.1325 mmol). The flask was fitted with a balloon filled with hydrogen gas. The reaction mixture was heated to 70° C. for 16 hours. After cooling the solution was filtered through Celite and concentrated in vacuo. The crude residue was separated by flash column chromatography on silica gel (ethyl acetate in hexanes) to afford an over-reduced product.

Stage 2: The product from Stage 1 was dissolved in Acetonitrile (120 mL) and acetic acid (20 mL) to which Manganese dioxide (11.5 g, 132 mmol) was added. The solution was stirred for 16 h at 23° C. The reaction mixture was filtered through Celite and solvent removed in vacuo.

Stage 3: To the crude product from Stage 2 in acetic acid (40 mL) was added N-Iodosuccinimide (3.0 g, 13 mmol). The reaction mixture was heated to 65° C. for 16 hours. The solvent was removed in vacuo. The crude was dissolved in an abundance of ethyl acetate and filtered. The filtrate was concentrated in vacuo and the crude residue was subjected to flash column chromatography on silica gel (eluent: ethyl acetate in hexanes) to afford 4-(2,6-dimethylphenyl)-5-iodo-pyrimidin-2-amine (3.34 g, 77%) as an orange solid. ESI-MS m/z calc. 325.0076, found 326.26 (M+1)⁺; Retention time: 0.57 minutes; LC method D.

### Step 2: N-[4-(2,6-Dimethylphenyl)-5-iodo-pyrimidin-2-yl]-3-nitro-benzenesulfonamide

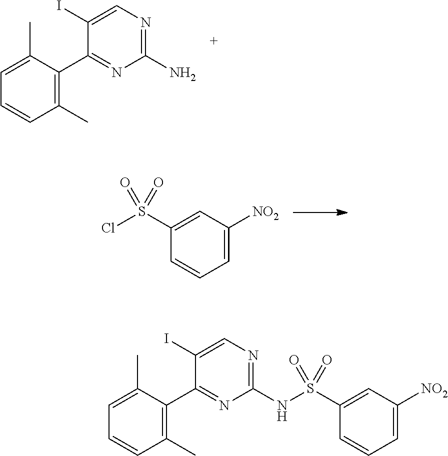 [SMILES: Cc1cccc(C)c1-c1nc(N)ncc1I ; O=[N+]([O-])c1cccc(S(=O)(=O)Cl)c1 ; Cc1cccc(C)c1-c1nc(NS(=O)(=O)c2cccc([N+](=O)[O-])c2)ncc1I]

To a solution of 4-(2,6-dimethylphenyl)-5-iodo-pyrimidin-2-amine (3.34 g, 10.3 mmol) in DMF (41 mL) at 0° C. was added sodium hydride (1.6 g of 60% w/w, 40.00 mmol). After 5 minutes the reaction was allowed to stir at 23° C. for 10 minutes. The reaction was cooled to 0° C. before adding 3-nitrobenzenesulfonyl chloride (4.6 g, 20.76 mmol) in one portion. After 15 minutes, the reaction was acidified with acetic acid (8.8 mL, 154.7 mmol) and diluted with water and ethyl acetate/hexanes (1:1). The organic layer was separated, and the aqueous layer was further extracted with ethyl acetate/hexanes (1:1, 4×). The combined organics were washed twice with brine, dried with magnesium sulfate, filtered and concentrated in vacuo. The crude residue was separated by flash column chromatography on silica gel (ethyl acetate in hexanes) to afford N-[4-(2,6-dimethylphenyl)-5-iodo-pyrimidin-2-yl]-3-nitro-benzenesulfonamide (1.58 g, 21%) as a yellow solid. ESI-MS m/z calc. 509.98587, found 511.32 (M+1)⁺; Retention time: 0.69 minutes; LC method D.

### Step 3: 6-[2-[(3-Aminophenyl)sulfonylamino]-4-(2,6-dimethylphenyl)pyrimidin-5-yl]hexanoic acid (Compound 416)

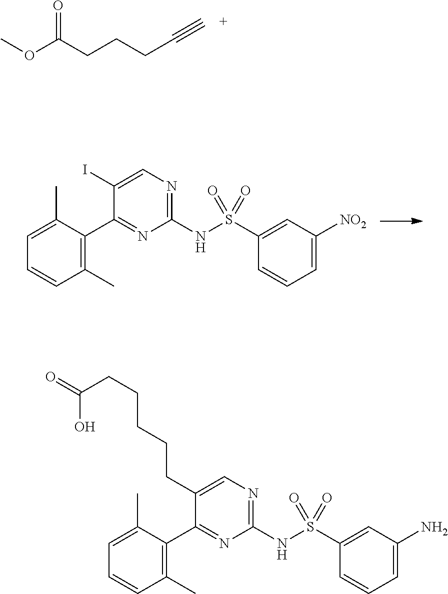

Stage 1: A heterogeneous solution consisting of N-[4-(2,6-dimethylphenyl)-5-iodo-pyrimidin-2-yl]-3-nitro-benzenesulfonamide (411 mg, 0.5557 mmol), methyl hex-5-ynoate (84.1 mg, 0.6667 mmol), triethylamine (387 μL, 2.777 mmol), copper(I) iodide (6.35 mg, 0.03334 mmol), and tetrakis(triphenylphosphine)palladium(0) (38.5 mg, 0.03332 mmol) in DMSO (3.0 mL) was heated to 80° C. in a sealed vial for 24 hours. The crude reaction mixture was directly separated by HPLC (acetonitrile in water with 0.1% hydrochloric acid).

Stage 2: The residue isolated from stage one was dissolved in ethanol (28.4 mL) and palladium on carbon (591 mg of 10% w/w, 0.5553 mmol) was added. Acetic acid (33.4 mg, 0.5562 mmol) was added. A hydrogen balloon was fixed to the top of the reaction vessel. The reaction was stirred for 16 hours. The solvent was removed in vacuo and the crude residue was separated by flash column chromatography on silica gel (ethyl acetate in hexanes) to afford a yellow solid.

Stage 3: To the product from stage 2 dissolved in acetic acid (2.4 mL) was added Manganese (IV) oxide (96.6 mg, 1.111 mmol) (amounts based on 0.20 mmol). The reaction was stirred for 1 hour at 23° C. The solution was filtered and concentrated in vacuo.

Stage 4: To the crude residue in THE (2.4 mL) and water (2.4 mL) was added NaOH (44.4 mg, 1.110 mmol). The solution was warmed to 60° C. for 2 hours before acidifying with hydrochloric acid (137 μL of 37% w/v, 1.390 mmol) and further diluting with diethyl ether. The organic layer was separated and the aqueous layer was extracted with diethyl ether (4×). The combined organics were washed with brine, dried over magnesium sulfate, filtered and concentrated in vacuo. The crude residue was separated by HPLC (acetonitrile in water with 0.1% hydrochloric acid) to afford 6-[2-[(3-aminophenyl)sulfonylamino]-4-(2,6-dimethylphenyl)pyrimidin-5-yl]hexanoic acid (10 mg, 4%) as a white solid (93% purity by UV-VIS). ESI-MS m/z calc. 468.18314, found 469.51 (M+1)⁺; Retention time: 1.4 minutes; LC method A.

### Example 136: Preparation of Compound 417 and Compound 418

### Step 1: N-[4-(4-methylpyrazol-1-yl)-6-phenoxy-pyrimidin-2-yl]benzenesulfonamide Compound 417) and N-[4,6-bis(4-methylpyrazol-1-yl)pyrimidin-2-yl]benzenesulfonamide (Compound 418)

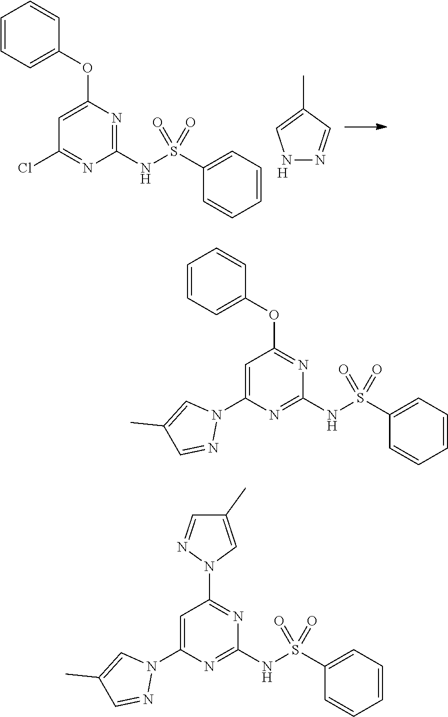

To a solution of 4-methyl-1H-pyrazole (approximately 12.32 mg, 0.1500 mmol) in DMF (0.4 mL) at 0° C. was added NaH (approximately 7.999 mg of 60% w/w, 0.2000 mmol) and the reaction mixture stirred at this temperature for 15 minutes. N-(4-chloro-6-phenoxy-pyrimidin-2-yl)benzenesulfonamide (18.09 mg, 0.05 mmol) was added to the reaction mixture, the cooling bath removed and the reaction mixture stirred at room temperature for 1 hour. The reaction mixture was diluted with MeOH, filtered and purification by HPLC (1-99% ACN in water (HCl modifier)) gave both N-[4-(4-methylpyrazol-1-yl)-6-phenoxy-pyrimidin-2-yl]benzenesulfonamide (2.8 mg) (ESI-MS m/z calc. 407.10522, found 408.5 (M+1)⁺; Retention time: 1.8 minutes (LC method A) and N-[4,6-bis(4-methylpyrazol-1-yl)pyrimidin-2-yl]benzenesulfonamide (11.7 mg) (ESI-MS m/z calc. 395.11646, found 396.5 (M+1)⁺; Retention time: 1.68 minutes (LC method A).

### Example 137: Characterization of Compounds 419-424

The compounds in the following tables were prepared in a manner analogous to that described above using commercially available reagents and intermediates described herein.

| | | LCMS | | | |
|---|---|---|---|---|---|
| | | Room | | | |
| Compound | | Temperature | Calc. | | LCMS |
| number | Structure | (min) | mass | M + 1 | method |
| 419 | 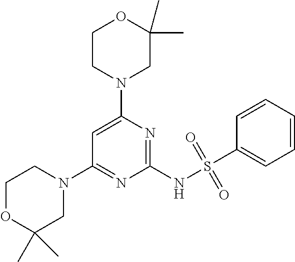 [SMILES: CC1(C)CN(c2cc(N3CCOC(C)(C)C3)nc(NS(=O)(=O)c3ccccc3)n2)CCO1] | 1.85 | 461.21 | 462.6 | A |
| 420 | 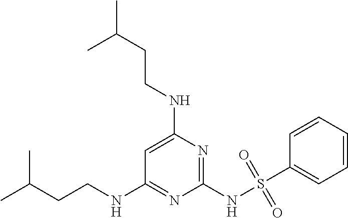 [SMILES: CC(C)CCNc1cc(NCCC(C)C)nc(NS(=O)(=O)c2ccccc2)n1] | 1.58 | 405.22 | 406.5 | A |
| 421 | 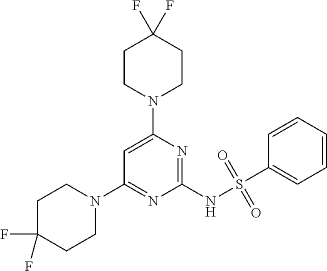 [SMILES: O=S(=O)(Nc1nc(N2CCC(F)(F)CC2)cc(N2CCC(F)(F)CC2)n1)c1ccccc1] | 1.63 | 473.151 | 474.5 | A |
| 422 | 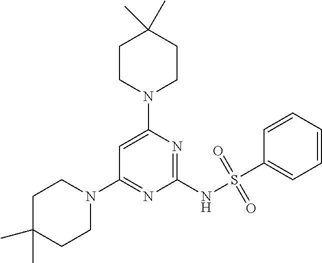 [SMILES: CC1(C)CCN(c2cc(N3CCC(C)(C)CC3)nc(NS(=O)(=O)c3ccccc3)n2)CC1] | 1.84 | 457.251 | 458.6 | A |
| 423 | 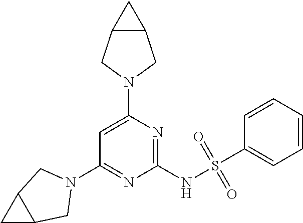 [SMILES: O=S(=O)(Nc1nc(N2CC3CC3C2)cc(N2CC3CC3C2)n1)c1ccccc1] | 1.35 | 397.157 | 398.5 | A |
| 424 | 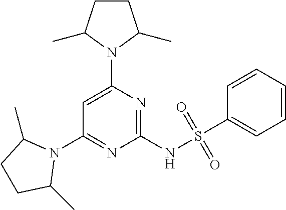 [SMILES: CC1CCC(C)N1c1cc(N2C(C)CCC2C)nc(NS(=O)(=O)c2ccccc2)n1] | 1.6 | 429.22 | 430.5 | A |

| Compound | |
|---|---|
| number | NMR |
| 412 | ¹H NMR (400 MHz, DMSO-d₆) δ 10.73 (s, 1H), 7.85 (s, 2H), 7.58 (d, J = |
| | 18.8 Hz, 3H), 5.49 (s, 1H), 3.56 (s, 4H), 3.38 (t, J = 5.1 Hz, 4H), 3.23 (s, |
| | 4H, 1.06 (s, 12H). |

### Example 138: Preparation of Compound 425

### Step 1: N-[14-chloro-5-ethyl-6-(2-isobutylphenyl)pyrimidin-2-yl]-1-methyl-pyrazole-4-sulfonamide

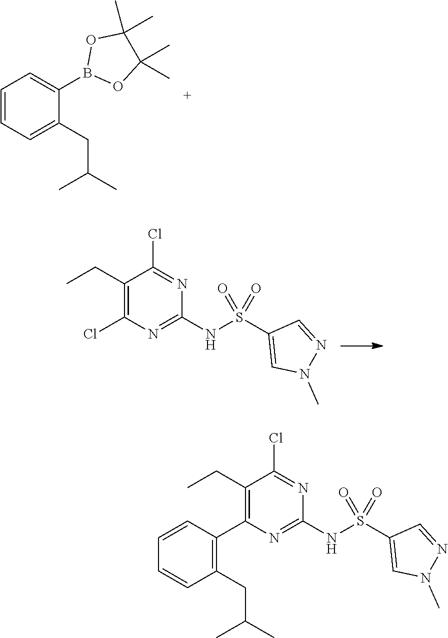 [SMILES: CC(C)Cc1ccccc1B1OC(C)(C)C(C)(C)O1 ; CCc1c(Cl)nc(NS(=O)(=O)c2cnn(C)c2)nc1Cl ; CCc1c(Cl)nc(NS(=O)(=O)c2cnn(C)c2)nc1-c1ccccc1CC(C)C]

To a solution of N-(4,6-dichloro-5-ethyl-pyrimidin-2-yl)-1-methyl-pyrazole-4-sulfonamide (2.07 g, 6.157 mmol), 2-(2-isobutylphenyl)-4,4,5,5-tetramethyl-1,3,2-dioxaborolane (1.8 g, 6.918 mmol) and Pd(dppf)C₁₂ (0.48 g, 0.6560 mmol) was added K₂CO₃ (9 mL of 2 M, 18.00 mmol) and the mixture purged with nitrogen for 5 minutes. The reaction mixture was stirred at 80° C. for 16 hours, poured into water, the pH adjusted to ˜4 with 1 N HCl and extracted with EtOAc (3×). Organics combined, washed with water (2×), brine, dried over sodium sulfate and purification by column chromatography (80 g silica 10-70% EtOAc in hexanes) gave a white solid, N-[4-chloro-5-ethyl-6-(2-isobutylphenyl)pyrimidin-2-yl]-1-methyl-pyrazole-4-sulfonamide (1.3 g, 34%). ESI-MS m/z calc. 433.1339, found 434.2 (M+1)⁺; Retention time: 0.77 minutes; LC method A.

### Step 2: N-[4-[2,3-Dichloro-5-(4-methylpiperazin-1-yl)phenoxy]-5-ethyl-6-(2-isobutylphenyl)pyrimidin-2-yl]-1-methyl-pyrazole-4-sulfonamide (Compound 425)

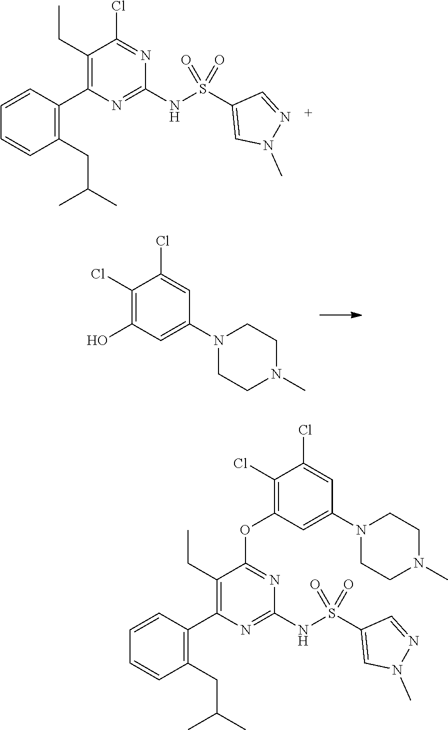

To a mixture of N-[4-chloro-5-ethyl-6-(2-isobutylphenyl)pyrimidin-2-yl]-1-methyl-pyrazole-4-sulfonamide (43.40 mg, 0.1 mmol) and Cs₂CO₃ (approximately 130.3 mg, 0.4000 mmol) in NMP (0.4 mL) was added 2,3-dichloro-5-(4-methylpiperazin-1-yl)phenol (approximately 78.34 mg, 0.3000 mmol) and the reaction mixture stirred at 120° C. for 16 hours. The reaction mixture was diluted with MeOH, filtered and purification by HPLC (1-99% ACN in water (HCl modifier)) gave N-[4-[2,3-dichloro-5-(4-methylpiperazin-1-yl)phenoxy]-5-ethyl-6-(2-isobutylphenyl)pyrimidin-2-yl]-1-methyl-pyrazole-4-sulfonamide (hydrochloride salt) (9.4 mg, 14%). ESI-MS m/z calc. 657.20557, found 658.3 (M+1)⁺; Retention time: 1.61 minutes; LC method A.

### Example 139: Preparation of Compound 426

### Step 1: N-[5-ethyl-4-(2-isobutylphenyl)-6-[4-(1-methyl-4-piperidyl)phenoxy]pyrimidin-2-yl]-1-methyl-pyrazole-4-sulfonamide

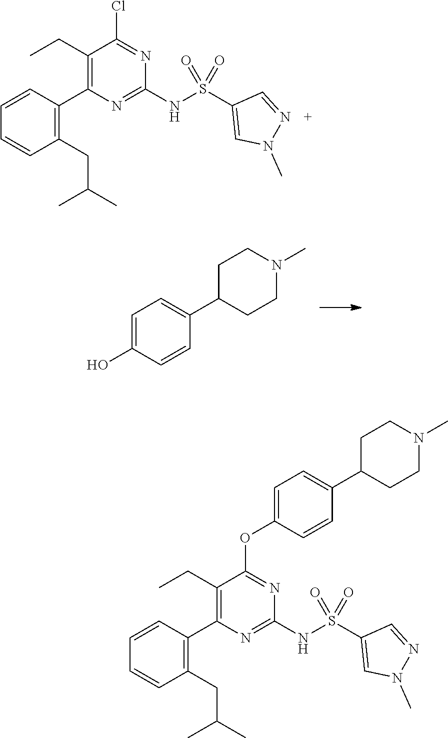 [SMILES: CCc1c(Cl)nc(NS(=O)(=O)c2cnn(C)c2)nc1-c1ccccc1CC(C)C ; CN1CCC(c2ccc(O)cc2)CC1 ; CCc1c(Oc2ccc(C3CCN(C)CC3)cc2)nc(NS(=O)(=O)c2cnn(C)c2)nc1-c1ccccc1CC(C)C]

A NMP (0.7 mL) mixture of N-[4-chloro-5-ethyl-6-(2-isobutylphenyl)pyrimidin-2-yl]-1-methyl-pyrazole-4-sulfonamide (20 mg, 0.04609 mmol), Cs₂CO₃ (approximately 60.08 mg, 0.1844 mmol), and 4-(1-methyl-4-piperidyl)phenol (acetate salt) (approximately 34.76 mg, 0.1383 mmol) was heated to 110° C. for 16 hours and then cooled to room temperature. The solution was filtered and the filtrate dissolved in 0.8 mL MeOH, and purified by reverse phase chromatography using a 15 min gradient of 20% MeCN in water to 80% MeCN (HCl modifier) to give N-[5-ethyl-4-(2-isobutylphenyl)-6-[4-(1-methyl-4-piperidyl)phenoxy]pyrimidin-2-yl]-1 -methyl-pyrazole-4-sulfonamide (hydrochloride salt) (2.2 mg, 5%). ESI-MS m/z calc. 588.28827, found 589.38 (M+1)⁺; Retention time: 1.52 minutes; LC method A.

### Example 140: Preparation of Compound 427

### Step 1: N-[4-[3-chloro-2-methyl-5-(4-methylpiperazin-1-yl)phenoxy]-5-ethyl-6-(2-isobutylphenyl)pyrimidin-2-yl]-1-methyl-pyrazole-4-sulfonamide

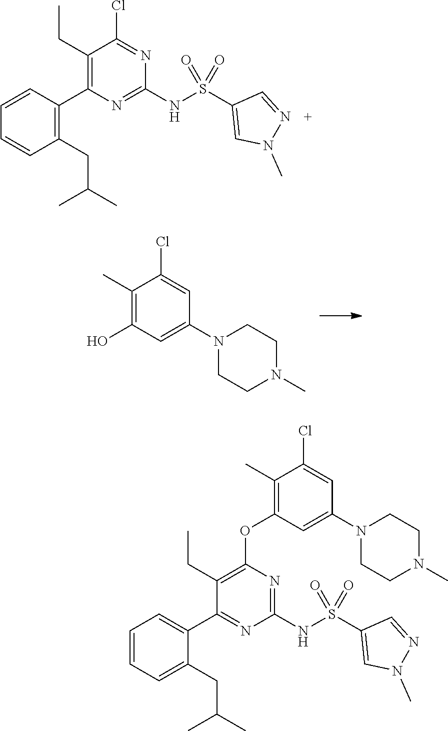

To a mixture of N-[4-chloro-5-ethyl-6-(2-isobutylphenyl)pyrimidin-2-yl]-1-methyl-pyrazole-4-sulfonamide (43.40 mg, 0.1 mmol) and Cs₂CO₃ (approximately 130.3 mg, 0.4000 mmol) in NMP (0.4 mL) was added 3-chloro-2-methyl-5-(4-methylpiperazin-1-yl)phenol (approximately 72.22 mg, 0.3000 mmol) and the reaction mixture was stirred at 120° C. for 16 hours. The reaction mixture was diluted with MeOH, filtered and purification by HPLC (1-99% ACN in water (HCl modifier)) gave N-[4-[3-chloro-2-methyl-5-(4-methylpiperazin-1-yl)phenoxy]-5-ethyl-6-(2-isobutylphenyl)pyrimidin-2-yl]-1-methyl-pyrazole-4-sulfonamide (hydrochloride salt) (12.3 mg, 18%). ESI-MS m/z calc. 637.2602, found 638.4 (M+1)⁺; Retention time: 1.58 minutes; LC method A.

### Example 141: Preparation of Compound 428

### Step 1: 3-(4-Methylpiperazin-1-yl)phenol

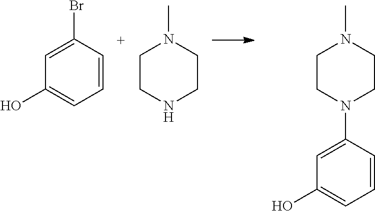

In a glass vial were 3-bromophenol (51.9 mg, 0.300 mmol), [2-(2-aminoethyl)phenyl]-chloro-palladium;di tert-butyl-[2-(2,4,6-triisopropylphenyl)phenyl]phosphane (26.5 mg, 0.0386 mmol), dioxane (800 μL), 1-methylpiperazine (100 μL), and sodium tert-butoxide (60.4 mg, 0.628 mmol) combined and the mixture was sparged under nitrogen for 5 minutes and then stirred at 35° C. for 30 minutes. The solution was filtered and the resulting residue dissolved in 1.2 mL DMSO/MeOH (1:1), and purified by reverse phase chromatography using a 15 minutes gradient of 1% MeCN in water to 99% MeCN (HCl modifier) to give 3-(4-methylpiperazin-1-yl)phenol (hydrochloride salt) (66.3 mg, 97%). ESI-MS m/z calc. 192.12627, found 193.29 (M+1)⁺; Retention time: 0.63 minutes; LC method A.

### Step 2: N-[5-ethyl-4-(2-isobutylphenyl)-6-[3-(4-methylpiperazin-1-yl)phenoxy]pyrimidin-2-yl]-1-methyl-pyrazole-4-sulfonamide

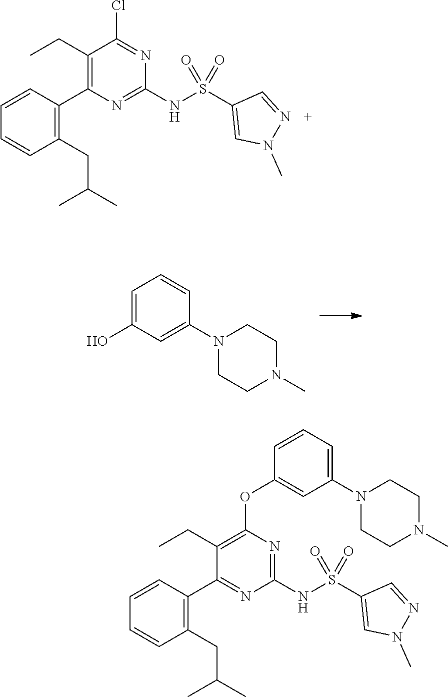

A NMP (0.7 mL) mixture of N-[4-chloro-5-ethyl-6-(2-isobutylphenyl)pyrimidin-2-yl]-1-methyl-pyrazole-4-sulfonamide (20 mg, 0.04609 mmol), Cs₂CO₃ (approximately 60.08 mg, 0.1844 mmol), and 3-(4-methylpiperazin-1-yl)phenol (hydrochloride salt) (approximately 31.63 mg, 0.1383 mmol) was heated to 110° C. for 16 hours and then cooled to room temperature. The solution was filtered and the filtrate dissolved in 0.8 mL MeOH, and purified by reverse phase chromatography using a 15 minutes gradient of 20% MeCN in water to 80% MeCN (HCl modifier) to give N-[5-ethyl-4-(2-isobutylphenyl)-6-[3-(4-methylpiperazin-1-yl)phenoxy]pyrimidin-2-yl]-1-methyl-pyrazole-4-sulfonamide (hydrochloride salt) (2.5 mg, 9%). ESI-MS m/z calc. 589.2835, found 590.37 (M+1)⁺; Retention time: 1.5 minutes; LC method A.

### Example 142: Preparation of Compound 429, Compound 430, and Compound 431

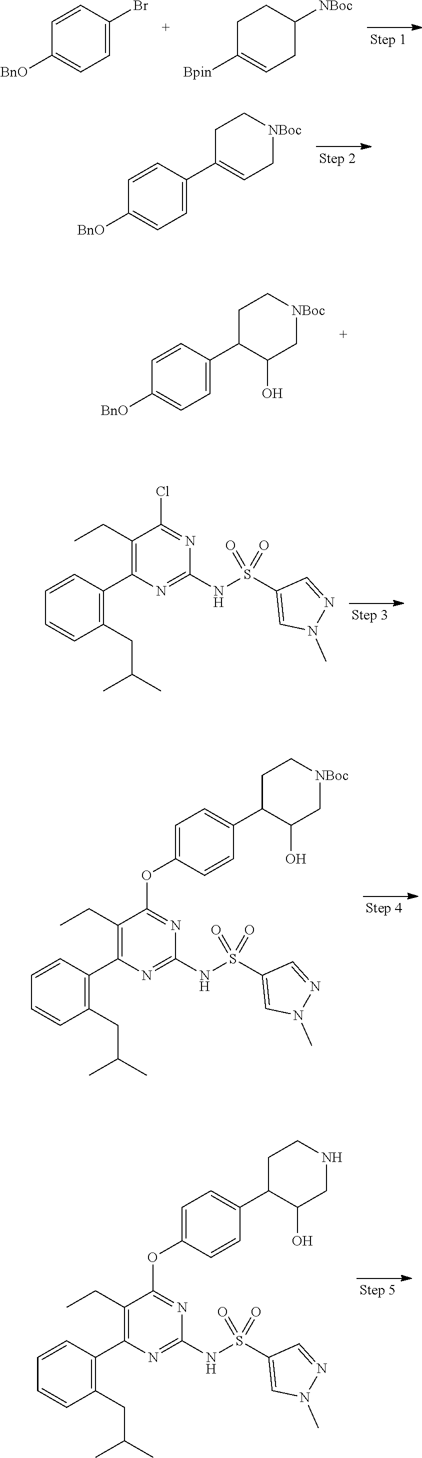

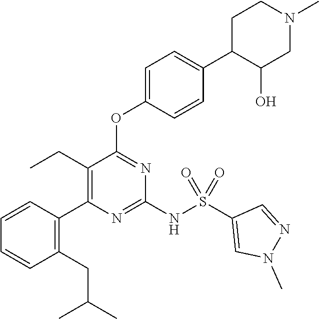 [SMILES: CCc1c(Oc2ccc(C3CCN(C)CC3O)cc2)nc(NS(=O)(=O)c2cnn(C)c2)nc1-c1ccccc1CC(C)C]

### Step 1: tert-Butyl 4-(4-benzyloxyphenyl)-3,6-dihydro-2H-pyridine-1-carboxylate

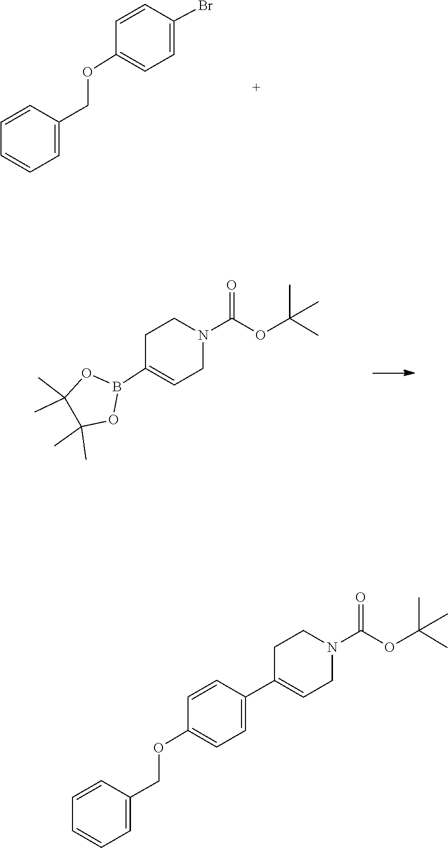 [SMILES: Brc1ccc(OCc2ccccc2)cc1 ; CC(C)(C)OC(=O)N1CC=C(B2OC(C)(C)C(C)(C)O2)CC1 ; CC(C)(C)OC(=O)N1CC=C(c2ccc(OCc3ccccc3)cc2)CC1]

To a solution of 1-benzyloxy-4-bromo-benzene (2.8 g, 10.64 mmol), tert-butyl 4-(4,4,5,5-tetramethyl-1,3,2-dioxaborolan-2-yl)-3,6-dihydro-2H-pyridine-1-carboxylate (3.38 g, 10.93 mmol) and bis(triphenylphosphine)palladium(II) dichloride(383 mg, 0.546 mmol) in DME (50 mL) and water (20 mL) was added sodium carbonate (3.77 g, 35.57 mmol) and the reaction mixture was stirred at 80° C. for 6 hours. The reaction mixture was filtered through Celite, poured into water and extracted with EtOAc (3×). The organics were combined, washed with water then brine, dried over Na₂SO₄, and evaporated to dryness. Purification by column chromatography (120 g silica; 0-20% EtOAc in hexanes) gave a white solid, tert-butyl 4-(4-benzyloxyphenyl)-3,6-dihydro-2H-pyridine-1-carboxylate (2.48 g, 62%). ESI-MS m/z calc. 365.1991, found 366.3 (M+1)⁺; Retention time: 0.82 minutes; LC method D.

### Step 2: tert-Butyl 4-(4-benzyloxyphenyl)-3-hydroxy-piperidine-1-carboxylate

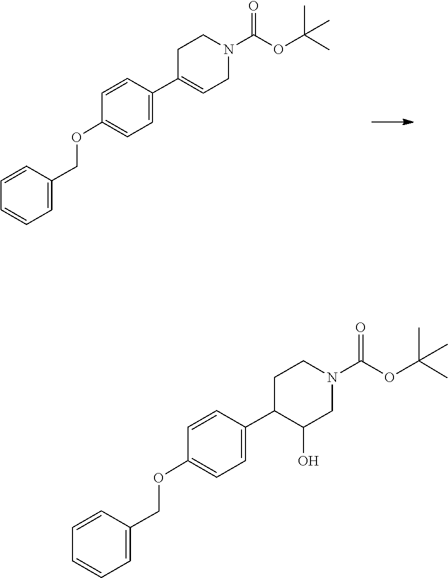 [SMILES: CC(C)(C)OC(=O)N1CC=C(c2ccc(OCc3ccccc3)cc2)CC1 ; CC(C)(C)OC(=O)N1CCC(c2ccc(OCc3ccccc3)cc2)C(O)C1]

To a solution of borane dimethylsulfide(approximately 1.658 mL of 2 M, 3.317 mmol) in THE (10.10 mL) at 0° C. was added a solution of tert-butyl 4-(4-benzyloxyphenyl)-3,6-dihydro-2H-pyridine-1-carboxylate (1.01 g, 2.764 mmol) in THE (5 mL) dropwise. The cooling bath was removed and stirred at room temperature for 2 hours. The reaction mixture was cooled to 0° C. and NaOH (approximately 1.013 mL of 3 M, 3.040 mmol), H₂O₂ (approximately 1.034 mL of 20% w/v, 6.081 mmol) and EtOH (1 mL) was added sequentially. The cooling bath was removed and the reaction mixture was stirred at 60° C. for 4 hours. The reaction mixture was poured into water and extracted with EtOAC (3×). The organic were combined, washed with water and brine, dried over Na₂SO₄, filtered through a short plug of silica and evaporated to dryness to give a white solid, tert-butyl 4-(4-benzyloxyphenyl)-3-hydroxy-piperidine-1-carboxylate (990 mg, 93%). ESI-MS m/z calc. 383.20966, found 384.3 (M+1)⁺; Retention time: 0.71 minutes; LC method D.

### Step 3:tert-butyl 4-[4-[5-ethyl-6-(2-isobutylphenyl)-2-[(1-methylpyrazol-4-yl)sulfonylamino]pyrimidin-4-yl]oxyphenyl]-3-hydroxy-piperidine-1-carboxylate (Compound 429)

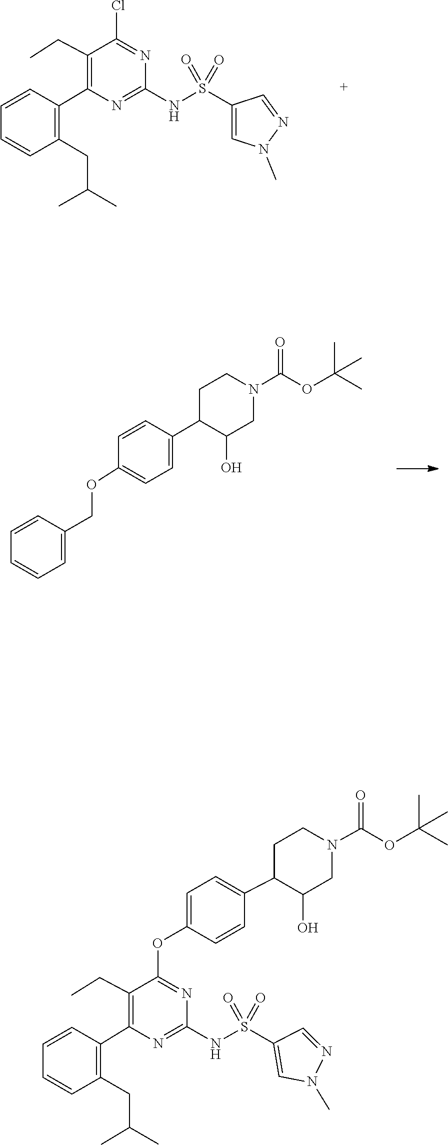 [SMILES: CCc1c(Cl)nc(NS(=O)(=O)c2cnn(C)c2)nc1-c1ccccc1CC(C)C ; CC(C)(C)OC(=O)N1CCC(c2ccc(OCc3ccccc3)cc2)C(O)C1 ; CCc1c(Oc2ccc(C3CCN(C(=O)OC(C)(C)C)CC3O)cc2)nc(NS(=O)(=O)c2cnn(C)c2)nc1-c1ccccc1CC(C)C]

To a solution of tert-butyl 4-(4-benzyloxyphenyl)-3-hydroxy-piperidine-1-carboxylate (360 mg, 0.9388 mmol) in EtOH (3.5 mL) was added Pd/C (20 mg of 10% w/w, 0.01879 mmol) and the reaction was stirred under a balloon of hydrogen for 3 hours. The reaction mixture was filtered through a plug of Celite and evaporated to dryness. The residue was taken up in NMP (4 mL) and to this solution was added N-[4-chloro-5-ethyl-6-(2-isobutylphenyl)pyrimidin-2-yl]-1 -methyl-pyrazole-4-sulfonamide (198 mg, 0.4563 mmol) and Cs₂CO₃ (520 mg, 1.596 mmol) and the reaction mixture was stirred at 120° C. for 16 hours. The reaction mixture was diluted with MeOH, filtered and purification by HPLC (1-99% ACN in water (HCl modifier)) gave tert-butyl 4-[4-[5-ethyl-6-(2-isobutylphenyl)-2-[(1-methylpyrazol-4-yl)sulfonylamino]pyrimidin-4-yl]oxyphenyl]-3-hydroxy-piperidine-1-carboxylate (154 mg, 23%). ESI-MS m/z calc. 690.31995, found 691.4 (M+1)⁺; Retention time: 2.19 minutes; LC method A.

### Step 4: N-[5-ethyl-4-[4-(3-hydroxy-4-piperidyl)phenoxy]-6-(2-isobutylphenyl)pyrimidin-2-yl]-1-methyl-pyrazole-4-sulfonamide (Compound 430)

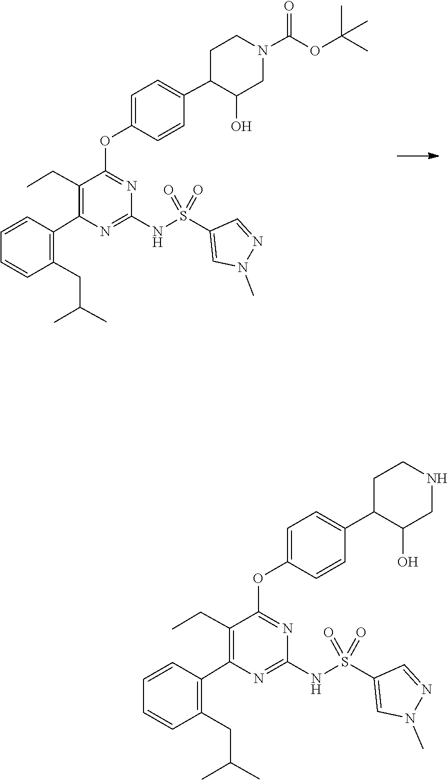 [SMILES: CCc1c(Oc2ccc(C3CCN(C(=O)OC(C)(C)C)CC3O)cc2)nc(NS(=O)(=O)c2cnn(C)c2)nc1-c1ccccc1CC(C)C ; CCc1c(Oc2ccc(C3CCNCC3O)cc2)nc(NS(=O)(=O)c2cnn(C)c2)nc1-c1ccccc1CC(C)C]

To a solution of tert-butyl 4-[4-[5-ethyl-6-(2-isobutylphenyl)-2-[(1-methylpyrazol-4-yl)sulfonylamino]pyrimidin-4-yl]oxyphenyl]-3-hydroxy-piperidine-1-carboxylate (20 mg, 0.02750 mmol) in DCM (0.4 mL) was added HCl (4M in dioxane) (140 μL of 4 M, 0.5600 mmol) and the reaction mixture stirred at room temperature for 20 minutes. The reaction mixture was diluted with MeOH, filtered and purification by HPLC (1-99% ACN in water (HCl modifier)) gave N-[5-ethyl-4-[4-(3-hydroxy-4-piperidyl)phenoxy]-6-(2-isobutylphenyl)pyrimidin-2-yl]-1-methyl-pyrazole-4-sulfonamide (hydrochloride salt) (2.6 mg, 15%). ESI-MS m/z calc. 590.2675, found 591.3 (M+1)⁺; Retention time: 1.45 minutes; LC method A.

### Step 5: N-[5-ethyl-4-[4-(3-hydroxy-1-methyl-4-piperidyl)phenoxy]-6-(2-isobutylphenyl)pyrimidin-2-yl]-1-methyl-pyrazole-4-sulfonamide (Compound 431)

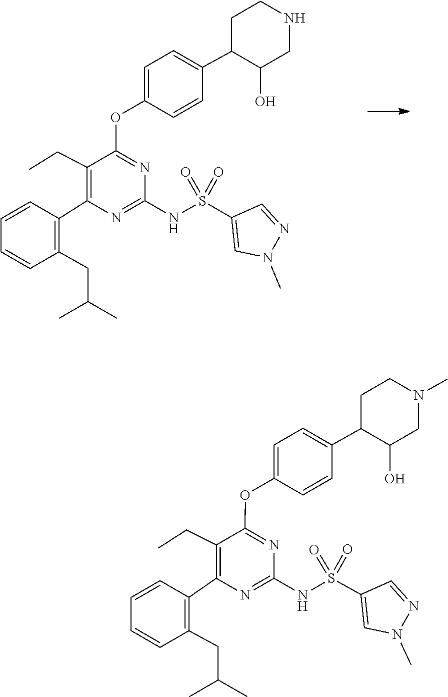 [SMILES: CCc1c(Oc2ccc(C3CCNCC3O)cc2)nc(NS(=O)(=O)c2cnn(C)c2)nc1-c1ccccc1CC(C)C ; CCc1c(Oc2ccc(C3CCN(C)CC3O)cc2)nc(NS(=O)(=O)c2cnn(C)c2)nc1-c1ccccc1CC(C)C]

A solution of tert-butyl 4-[4-[5-ethyl-6-(2-isobutylphenyl)-2-[(1-methylpyrazol-4-yl)sulfonylamino]pyrimidin-4-yl]oxyphenyl]-3-hydroxy-piperidine-1-carboxylate (50 mg, 0.06876 mmol), formic acid (200 μL, 5.301 mmol), formaldehyde (200 μL, 7.260 mmol) and MeOH (0.2 mL) was heated at 80° C. for 1 hour. The reaction mixture was diluted with MeOH, filtered and purification by HPLC (1-99% ACN in water (HCl modifier)) gave N-[5-ethyl-4-[4-(3-hydroxy-1-methyl-4-piperidyl)phenoxy]-6-(2-isobutylphenyl)pyrimidin-2-yl]-1-methyl-pyrazole-4-sulfonamide (hydrochloride salt) (5.9 mg, 13%). ESI-MS m/z calc. 604.2832, found 605.6 (M+1)⁺; Retention time: 1.36 minutes; LC method A.

### Example 143: Preparation of Compound 432

### Step 1: N-[5-ethyl-4-(2-isobutylphenyl)-6-[3-(1-methyl-4-piperidyl)phenoxy]pyrimidin-2-yl]-1-methyl-pyrazole-4-sulfonamide

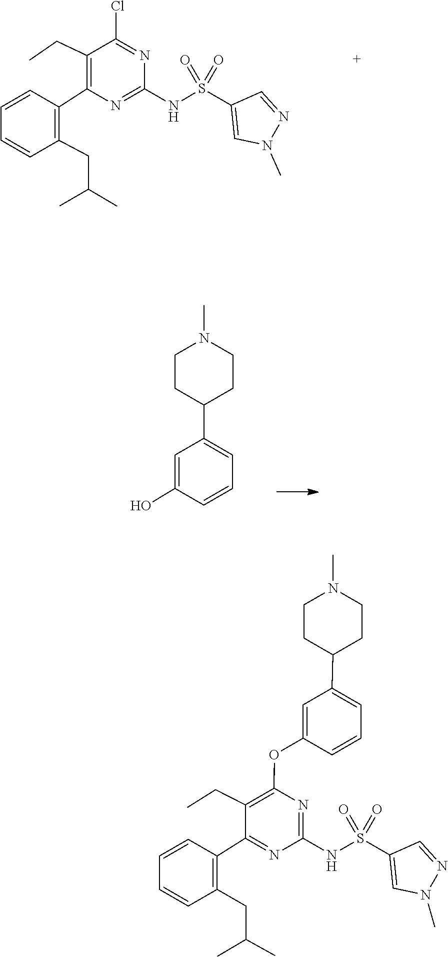 [SMILES: CCc1c(Cl)nc(NS(=O)(=O)c2cnn(C)c2)nc1-c1ccccc1CC(C)C ; CN1CCC(c2cccc(O)c2)CC1 ; CCc1c(Oc2cccc(C3CCN(C)CC3)c2)nc(NS(=O)(=O)c2cnn(C)c2)nc1-c1ccccc1CC(C)C]

A NMP (0.7 mL) mixture of N-[4-chloro-5-ethyl-6-(2-isobutylphenyl)pyrimidin-2-yl]-1-methyl-pyrazole-4-sulfonamide (20 mg, 0.04609 mmol), Cs₂CO₃ (approximately 60.08 mg, 0.1844 mmol), and 3-(1-methyl-4-piperidyl)phenol (approximately 26.45 mg, 0.1383 mmol) was heated at 110° C. for 16 hours and then cooled to room temperature. The solutions was filtered and the filtrate dissolved in 0.8 mL MeOH, and purified by reverse phase chromatography using a 15 minutes gradient of 20% MeCN in water to 80% MeCN (HCl modifier) to give N-[5-ethyl-4-(2-isobutylphenyl)-6-[3-(1-methyl-4-piperidyl)phenoxy]pyrimidin-2-yl]-1-methyl-pyrazole-4-sulfonamide (hydrochloride salt) (4.3mg, 3%). ¹H NMR (400 MHz, DMSO-d₆) δ 10.32 (s, 1H), 7.61-7.50 (m, 1H), 7.50-7.39 (m, 2H), 7.38-7.28 (m, 2H), 7.28-7.23 (m, 1H), 7.23-7.17 (m, 3H), 6.92 (s, 1H), 3.71 (s, 3H), 3.51-3.46 (m, 2H), 3.12-2.99 (m, 2H), 2.94-2.83 (m, 1H), 2.77 (d, J=4.8 Hz, 3H), 2.49-2.39 (m, 2H), 2.27-2.12 (m, 2H), 2.12-1.95 (m, 4H), 1.81-1.67 (m, 1H), 1.04 (t, J=7.4, 3.1 Hz, 3H), 0.79-0.69 (m, 6H). ESI-MS m/z calc. 588.28827, found 589.38 (M+1)⁺; Retention time: 1.52 minutes; LC method A.

### Example 144: Preparation of Compound 433

### Step 1: 2-Fluoro-5-(1-methyl-4-piperidyl)phenol

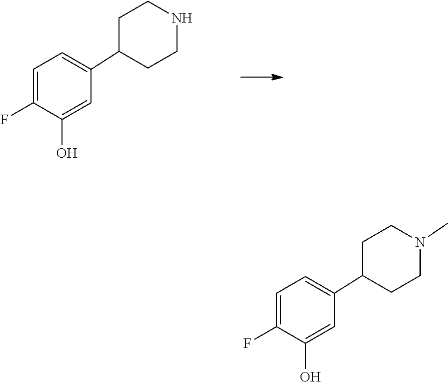

To a solution of 2-fluoro-5-(piperidin-4-yl)phenol hydrochloric acid salt (9.80 g, 42.31 mmol) in methanol (100 mL), triethylamine (10 mL) and 37% aqueous formaldehyde solution (40 mL, 480 mmol) were added 10% palladium on carbon (1.5 g, 1.41 mmol). The mixture was stirred under hydrogen atmosphere at 50 psi for 1 hour, filtered and concentrated. A saturated aqueous sodium bicarbonate solution (70 mL) and ethyl acetate (300 mL) were added The organic layer was washed with brine, dried over sodium sulfate and concentrated to give 2-fluoro-5-(N-methylpiperidin-4-yl)phenol(4.60 g, 52%) as an off-white solid. ¹H NMR (250 MHz, CDCl₃) δ (ppm): 7.00-6.50 (m, 3H), 3.10-2.95 (m, 2H), 2.40-2.20 (m, 1H), 2.34 (s, 3H), 2.15-2.05 (m, 2H), 1.80-1.65 (m, 4H). ESI-MS m/z calc. 209.1216, found 210.2 (M+1)⁺; Retention time: 2.72 minutes.

### Step 2: N-[5-ethyl-4-[2-fluoro-5-(1-methyl-4-piperidyl)phenoxy]-6-(2-isobutylphenyl)pyrimidin-2-yl]-1-methyl-pyrazole-4-sulfonamide

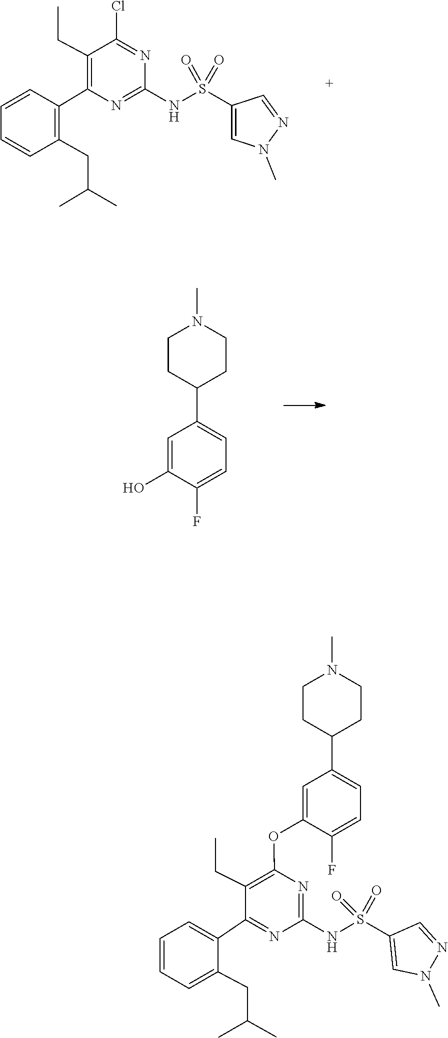 [SMILES: CCc1c(Cl)nc(NS(=O)(=O)c2cnn(C)c2)nc1-c1ccccc1CC(C)C ; CN1CCC(c2ccc(F)c(O)c2)CC1 ; CCc1c(Oc2cc(C3CCN(C)CC3)ccc2F)nc(NS(=O)(=O)c2cnn(C)c2)nc1-c1ccccc1CC(C)C]

To a mixture of N-[4-chloro-5-ethyl-6-(2-isobutylphenyl)pyrimidin-2-yl]-1-methyl-pyrazole-4-sulfonamide (43.40 mg, 0.1 mmol) and Cs₂CO₃ (approximately 130.3 mg, 0.4000 mmol) in NMP (0.4 mL) was added 2-fluoro-5-(1-methyl-4-piperidyl)phenol (approximately 62.78 mg, 0.3000 mmol) and the reaction mixture stirred at 120° C. for 16 hours. The reaction mixture was diluted with MeOH, filtered and purification by HPLC (1-99% ACN in water (HCl modifier)) gave N-[5-ethyl-4-[2-fluoro-5-(1-methyl-4-piperidyl)phenoxy]-6-(2-isobutylphenyl)pyrimidin-2-yl]-1-methyl-pyrazole-4-sulfonamide (hydrochloride salt) (23.9 mg, 39%). ESI-MS m/z calc. 606.2788, found 607.3 (M+1)⁺; Retention time: 1.46 minutes; LC method A.

### Example 145: Preparation of Compound 434

### Step 1: N-[5-ethyl-4-(2-isobutylphenyl)-6-[4-[(4-methylpiperazin-1-yl)methyl]phenoxy]pyrimidin-2-yl]-1-methyl-pyrazole-4-sulfonamide

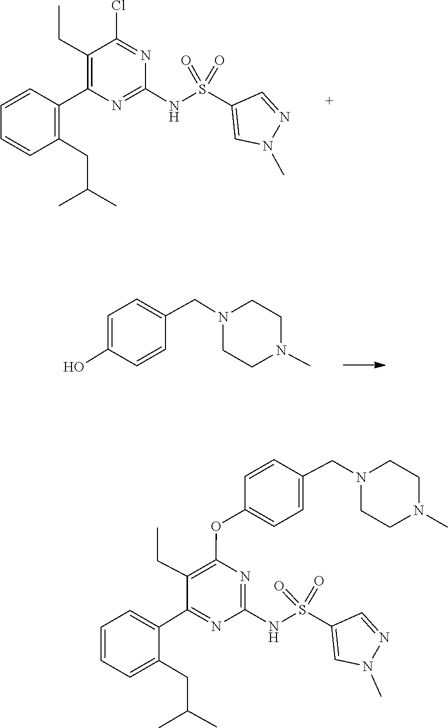 [SMILES: CCc1c(Cl)nc(NS(=O)(=O)c2cnn(C)c2)nc1-c1ccccc1CC(C)C ; CN1CCN(Cc2ccc(O)cc2)CC1 ; CCc1c(Oc2ccc(CN3CCN(C)CC3)cc2)nc(NS(=O)(=O)c2cnn(C)c2)nc1-c1ccccc1CC(C)C]

To a mixture of N-[4-chloro-5-ethyl-6-(2-isobutylphenyl)pyrimidin-2-yl]-1-methyl-pyrazole-4-sulfonamide (43.40 mg, 0.1 mmol) and Cs₂CO₃ (approximately 130.3 mg, 0.4000 mmol) in NMP (0.4 mL) was added 4-[(4-methylpiperazin-1-yl)methyl]phenol (approximately 61.89 mg, 0.3000 mmol) and the reaction mixture was stirred at 120° C. for 16 hours. The reaction mixture was diluted with MeOH, filtered and purification by HPLC (1-99% ACN in water (HCl modifier)) gave N-[5-ethyl-4-(2-isobutylphenyl)-6-[4-[(4-methylpiperazin-1-yl)methyl]phenoxy]pyrimidin-2-yl]-1-methyl-pyrazole-4-sulfonamide (hydrochloride salt) (8.2 mg, 13%). ESI-MS m/z calc. 603.29913, found 604.4 (M+1)⁺; Retention time: 1.29 minutes; LC method A.

### Example 146: Preparation of Compound 435

### Step 1: 5-(4-Methylpiperazin-1-yl)pyridin-3-ol

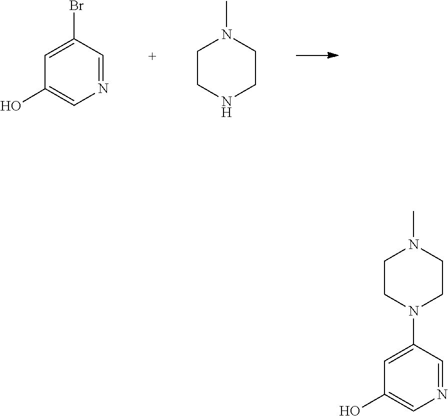

A stirred solution of 5-bromopyridin-3-ol (1 g, 5.747 mmol), 1-methylpiperazine (2 mL, 18.01 mmol), Chloro(2-di-t-butylphosphino-2′, 4′, 6′-tri-i-propyl-1,1′-biphenyl)[2-(2-aminoethyl)phenyl] palladium(II) [t-BuXPhos Palladacycle Gen. 1] (520 mg, 0.7573 mmol) and sodium tert-butoxide (1.3 g, 13.53 mmol) in dioxane (16 mL) was sparged under nitrogen for 5 minutes and then stirred at 45° C. for 16 hours. The reaction mixture was quenched with iN HCl and evaporated to dryness. The residue was taken up in 1:1 DMSO:MeOH, filtered. Purification by SFC (5-15% MeOH in C₀₂ (NH₃ modifier)) gave 5-(4-methylpiperazin-1-yl)pyridin-3-ol (240 mg, 22%). ESI-MS m/z calc. 193.1215, found 194.2 (M+1)⁺; Retention time: 0.07 minutes; LC method D.

### Step 2: N-[5-ethyl-4-(2-isobutylphenyl)-6-[[5-(4-methylpiperazin-1-yl)-3-pyridyl]oxy]pyrimidin-2-yl]-1-methyl-pyrazole-4-sulfonamide (Compound 435)

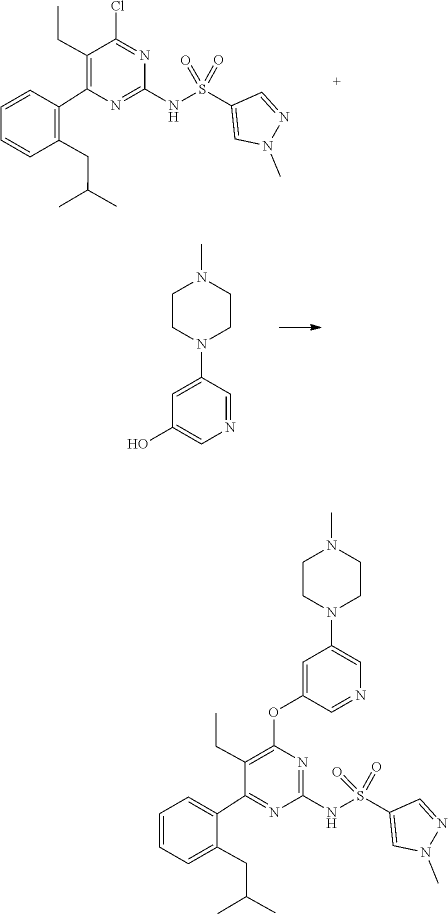 [SMILES: CCc1c(Cl)nc(NS(=O)(=O)c2cnn(C)c2)nc1-c1ccccc1CC(C)C ; CN1CCN(c2cncc(O)c2)CC1 ; CCc1c(Oc2cncc(N3CCN(C)CC3)c2)nc(NS(=O)(=O)c2cnn(C)c2)nc1-c1ccccc1CC(C)C]

To a mixture of N-[4-chloro-5-ethyl-6-(2-isobutylphenyl)pyrimidin-2-yl]-1-methyl-pyrazole-4-sulfonamide (43.40 mg, 0.1 mmol) and Cs₂CO₃ (approximately 130.3 mg, 0.4000 mmol) in NMP (0.4 mL) was added 5-(4-methylpiperazin-1-yl)pyridin-3-ol (approximately 68.91 mg, 0.3000 mmol) and the reaction mixture was stirred at 120° C. for 16 hours. The reaction mixture was diluted with MeOH, filtered and purification by HPLC (1-99% ACN in water (HCl modifier)) gave N-[5-ethyl-4-(2-isobutylphenyl)-6-[[5-(4-methylpiperazin-1-yl)-3-pyridyl]oxy]pyrimidin-2-yl]-1-methyl-pyrazole-4-sulfonamide (hydrochloride salt) (32.2 mg, 55%). ESI-MS m/z calc. 590.27875, found 591.4 (M+1)⁺; Retention time: 1.19 minutes; LC method A.

### Example 147: Preparation of Compound 436

### Step 1: 6-(4-Methylpiperazin-1-yl)pyridin-2-ol

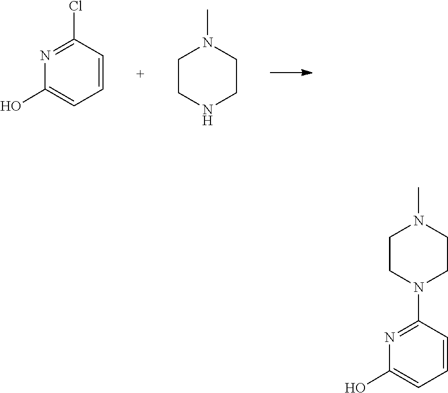

A stirred solution of 1-methylpiperazine (2 mL, 18.01 mmol), 6-chloropyridin-2-ol (1.01 g, 7.797 mmol), Chloro(2-di-t-butylphosphino-2′, 4′, 6′-tri-i-propyl-1,1′-biphenyl)[2-(2-aminoethyl)phenyl] palladium(II) [t-BuXPhos Palladacycle Gen. 1] (680 mg, 0.9903 mmol) and sodium tert-butoxide (1.81 g, 18.83 mmol) in dioxane (16 mL) was purged with nitrogen for 5 minutes, then heated at 45° C. for 1 hour. The reaction mixture was cooled, quenched with 1 N HCl and evaporated to dryness. The residue was taken up in 1:1 DMSO: MeOH, filtered and purification by HPLC (1-99% ACN in water (HCl modifier)) gave 6-(4-methylpiperazin-1-yl)pyridin-2-ol (380 mg, 25%). ESI-MS m/z calc. 193.1215, found 194.4 (M+1)⁺; Retention time: 0.07 minutes; LC method D.

### Step 2: N-[5-ethyl-4-(2-isobutylphenyl)-6-[[6-(4-methylpiperazin-1-yl)-2-pyridyl]oxy]pyrimidin-2-yl]-1-methyl-pyrazole-4-sulfonamide (Compound 436)

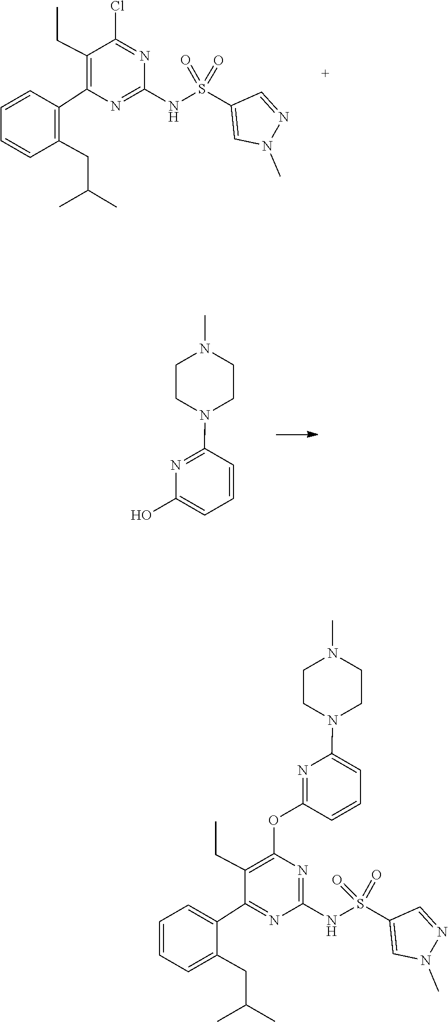 [SMILES: CCc1c(Cl)nc(NS(=O)(=O)c2cnn(C)c2)nc1-c1ccccc1CC(C)C ; CN1CCN(c2cccc(O)n2)CC1 ; CCc1c(Oc2cccc(N3CCN(C)CC3)n2)nc(NS(=O)(=O)c2cnn(C)c2)nc1-c1ccccc1CC(C)C]

To a mixture of N-[4-chloro-5-ethyl-6-(2-isobutylphenyl)pyrimidin-2-yl]-1-methyl-pyrazole-4-sulfonamide (43.40 mg, 0.1 mmol) and Cs₂CO₃ (approximately 130.3 mg, 0.4000 mmol) in NMP (0.4 mL) was added 6-(4-methylpiperazin-1-yl)pyridin-2-ol (approximately 57.97 mg, 0.3000 mmol) and the reaction mixture was stirred at 120° C. for 16 hours. The reaction mixture was diluted with MeOH, filtered and purification by HPLC (1-99% ACN in water (HCl modifier)) gave N-[5-ethyl-4-(2-isobutylphenyl)-6-[[6-(4-methylpiperazin-1-yl)-2-pyridyl]oxy]pyrimidin-2-yl]-1-methyl-pyrazole-4-sulfonamide (hydrochloride salt) (8.7 mg, 13%). ESI-MS m/z calc. 590.27875, found 591.4 (M+1)⁺; Retention time: 1.38 minutes; LC method A.

### Example 148: Preparation of Compound 437

### Step 1: 4-Fluoro-3-(1-methyl-4-piperidyl)phenol

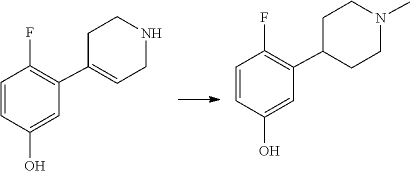

To a solution of 4-fluoro-3-(2,3,6-trihydropyridin-4-yl)phenol hydrochloric acid salt (7.6 g, 53.52 mmol) in methanol (100 mL) were added triethylamine (10 mL), a 37% aqueous formaldehyde solution (40 mL, 480 mmol) and 10% palladium on carbon (1.5 g). The mixture was stirred under hydrogen atmosphere at 50 psi for 1 hour. After filtration and concentration, saturated sodium bicarbonate (50 mL) and ethyl acetate (200 mL) were added and the two phases were separated. The organic layer was washed with brine, dried over sodium sulfate and concentrated. The crude residue was dissolved in methanol (50 mL). 10% Palladium on carbon (2.0 g) was added. The mixture was stirred under hydrogen atmosphere at 60 psi for 16 hours. The reaction mixture was filtered through Celite pad and the filtrate was concentrated to give 4-fluoro-3-(N-methylpiperidin-4-yl)phenol (4.5 g, 45%) as an off-white solid. ¹H NMR (250 MHz, CDCl₃) δ (ppm): 6.95-6.55 (m, 3H), 3.10-3.00 (m, 2H), 2.90-2.75 (m, 1H), 2.34 (s, 3H), 2.25-2.05 (m, 2H), 1.80-1.60 (m, 4H). ESI-MS m/z calc. 209.1216, found 210.2 (M+1)⁺; Retention time: 2.59 minutes.

### Step 2: N-[5-ethyl-4-[4-fluoro-3-(1-methyl-4-piperidyl)phenoxy]-6-(2-isobutylphenyl)pyrimidin-2-yl]-1-methyl-pyrazole-4-sulfonamide

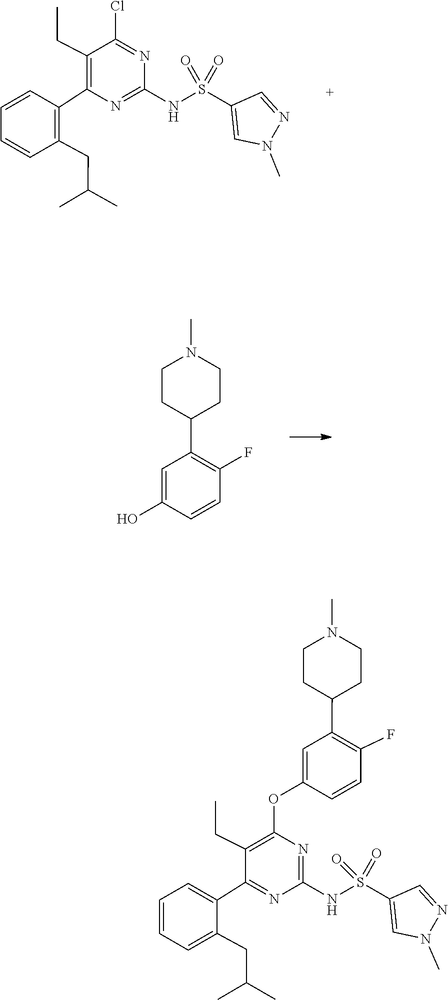 [SMILES: CCc1c(Cl)nc(NS(=O)(=O)c2cnn(C)c2)nc1-c1ccccc1CC(C)C ; CN1CCC(c2cc(O)ccc2F)CC1 ; CCc1c(Oc2ccc(F)c(C3CCN(C)CC3)c2)nc(NS(=O)(=O)c2cnn(C)c2)nc1-c1ccccc1CC(C)C]

To a mixture of N-[4-chloro-5-ethyl-6-(2-isobutylphenyl)pyrimidin-2-yl]-1-methyl-pyrazole-4-sulfonamide (43.40 mg, 0.1 mmol) and Cs₂CO₃ (approximately 130.3 mg, 0.4000 mmol) in NMP (0.4 mL) was added 4-fluoro-3-(1-methyl-4-piperidyl)phenol (approximately 62.78 mg, 0.3000 mmol) and the reaction mixture was stirred at 120° C. for 16 hours. The reaction mixture was diluted with MeOH, filtered and purification by HPLC (1-99% ACN in water (HCl modifier)) gave N-[5-ethyl-4-[4-fluoro-3-(1-methyl-4-piperidyl)phenoxy]-6-(2-isobutylphenyl)pyrimidin-2-yl]-1-methyl-pyrazole-4-sulfonamide (hydrochloride salt) (20.3 mg, 33%). ESI-MS m/z calc. 606.2788, found 607.3 (M+1)⁺; Retention time: 1.44 minutes; LC method A.

### Example 149: Preparation of Compound 438

### Step 1: N-[5-ethyl-4-[2-fluoro-3-(4-methylpiperazin-1-yl)phenoxy]-6-(2-isobutylphenyl)pyrimidin-2-yl]-1-methyl-pyrazole-4-sulfonamide

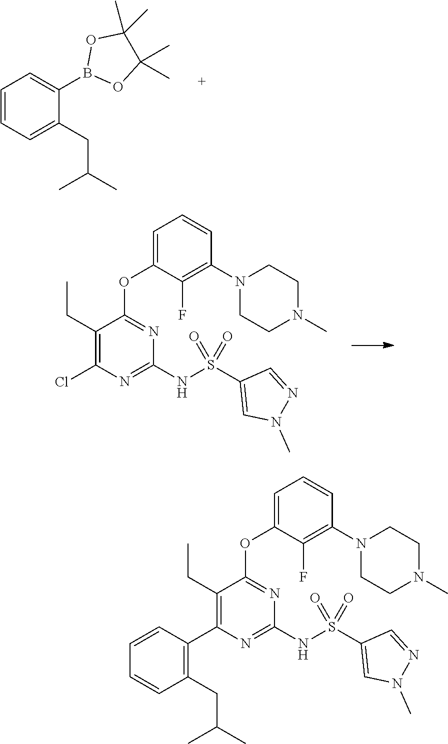

A dioxane (0.7 mL) mixture of 2-(2-isobutylphenyl)-4,4,5,5-tetramethyl-1,3,2-dioxaborolane (14.2 mg, 0.05458 mmol), N-[4-chloro-5-ethyl-6-[2-fluoro-3-(4-methylpiperazin-1-yl)phenoxy]pyrimidin-2-yl]-1-methyl-pyrazole-4-sulfonamide (hydrochloride salt) (20 mg, 0.03660 mmol), Pd(dppf)C₁₂ (5.6 mg, 0.006857 mmol), and potassium carbonate (100 μL of 2 M, 0.2000 mmol) was sparged with nitrogen for 1 minute and then microwaved at 120° C. for 30 minutes. The solution was filtered and the filtrate was purified by reverse phase chromatography using a 1-99% over 15 minutes gradient of MeCN in water (HCl modifier) to give N-[5-ethyl-4-[2-fluoro-3-(4-methylpiperazin-1-yl)phenoxy]-6-(2-isobutylphenyl)pyrimidin-2-yl]-1-methyl-pyrazole-4-sulfonamide (hydrochloride salt) (2.2 mg, 9%). ESI-MS m/z calc. 607.2741, found 608.38 (M+1)⁺; Retention time: 1.57 minutes; LC method A.

### Example 150: Preparation of Compound 439

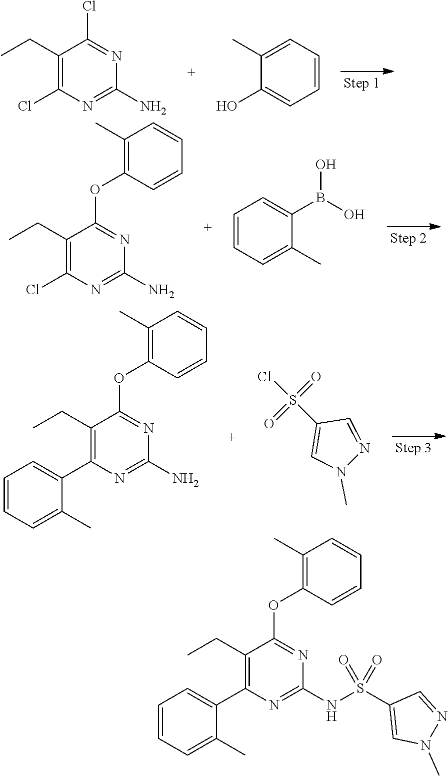

### Step 1: 4-Chloro-5-ethyl-6-(2-methylphenoxy)pyrimidin-2-amine

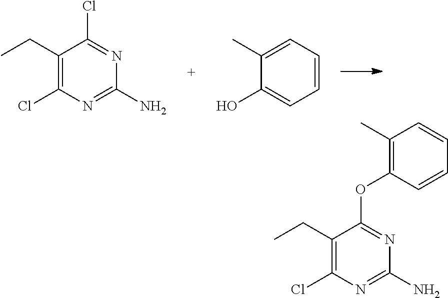

To 4,6-dichloro-5-ethyl-pyrimidin-2-amine (200 mg, 1.041 mmol) and potassium carbonate (432.5 mg, 3.129 mmol) was added DMF (2.5 mL) followed by o-cresol (204 μL, 1.043 mmol). The mixture was heated at 110° C. for 3 hours. EtOAc and water were added to the reaction. The two layers were separated after shaking. The aqueous layer was extracted with EtOAc (×1). The organic layer was washed with water (×5). The organic layer was dried over Na₂SO₄, filtered and the solvent was evaporated under reduced pressure to yield 4-chloro-5-ethyl-6-(2-methylphenoxy)pyrimidin-2-amine (275 mg, 100%) as a brown viscous solid. ESI-MS m/z calc. 263.08255, found 264.1 (M+1)⁺; Retention time: 0.69 minutes; LC method A.

### Step 2: 5-Ethyl-4-(2-methylphenoxy)-6-(o-tolyl)pyrimidin-2-amine

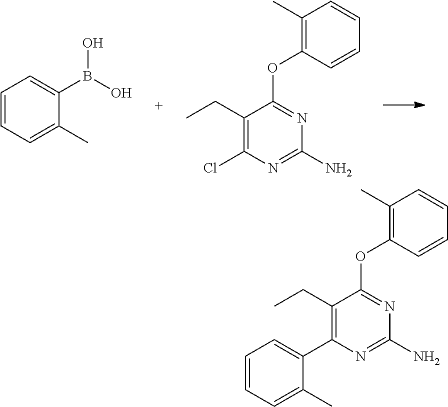

The mixture of 4-chloro-5-ethyl-6-(2-methylphenoxy)pyrimidin-2-amine (274.5 mg, 1.041 mmol), o-tolylboronic acid (154.9 mg, 1.139 mmol), Pd(dppf)C₁₂ (78.3 mg, 0.1070 mmol), and potassium carbonate (1.1 mL of 2 M, 2.200 mmol) in 1,2-dimethoxyethane (2.3 mL) was degassed by flow of nitrogen and the reaction was stirred at 130° C. for 1 hour in a pressure vessel. The reaction was filtered. EtOAc and water were added to the reaction and the two layers were separated. The organic layer was dried over Na₂SO₄, filtered and concentrated. The crude product was purified on 24 g of silica gel utilizing a gradient of 0-30% ethyl acetate in DCM to yield a yellow solid, 5-ethyl-4-(2-methylphenoxy)-6-(o-tolyl)pyrimidin-2-amine (175.6 mg, 53%). ¹H NMR (400 MHz, DMSO-d₆) δ 7.35-7.22 (m, 5H), 7.21-7.10 (m, 3H), 6.36 (s, 2H), 2.47-2.16 (m, 2H), 2.14 (s, 3H), 2.13 (s, 3H), 0.99 (t, J=7.4 Hz, 3H). ESI-MS m/z calc. 319.16846, found 320.3 (M+1)⁺; Retention time: 1.4 minutes; LC method A.

### Step 3: N-[5-ethyl-4-(2-methylphenoxy)-6-(o-tolyl)pyrimidin-2-yl]-1-methyl-pyrazole-4-sulfonamide (Compound 439)

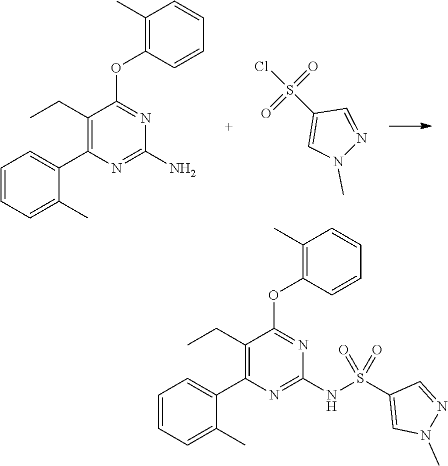

To 5-ethyl-4-(2-methylphenoxy)-6-(o-tolyl)pyrimidin-2-amine (50 mg, 0.1565 mmol), 1-methylpyrazole-4-sulfonyl chloride (126.2 mg, 0.6987 mmol) and pyridine (2.5 mL) were added and the reaction was stirred at 110° C. for 23 hours. Water and EtOAc were added to the reaction and the two layers were separated. The organic layer was washed with water (×2), dried over Na₂SO₄, filtered and the solvent was evaporated under reduced pressure. The compound was dissolved in DMSO, filtered and purified using a reverse phase HPLC C₁₈ column and a dual gradient run from 1-99% mobile phase B over 30 minutes (Mobile phase A=H₂O (5 mM HCl). Mobile phase B═CH₃CN) to yield a light yellow solid, N-[5-ethyl-4-(2-methylphenoxy)-6-(o-tolyl)pyrimidin-2-yl]-1-methyl-pyrazole-4-sulfonamide (35.4 mg, 49%).

¹H NMR (400 MHz, Acetonitrile-d₃) δ 9.38 (s, 1H), 7.46-7.41 (m, 1H), 7.41-7.28 (m, 5H), 7.26-7.15 (m, 3H), 7.05 (s, 1H), 3.73 (s, 3H), 2.59-2.28 (m, 2H), 2.20 (s, 3H), 2.13 (s, 3H), 1.05 (t, J=7.4 Hz, 3H). ESI-MS m/z calc. 463.16782, found 464.2 (M+1)⁺; Retention time: 1.81 minutes; LC method A.

### Example 151: Preparation of Compound 440

### Step 1: 4-Chloro-5-ethyl-6-(o-tolyl)pyrimidin-2-amine

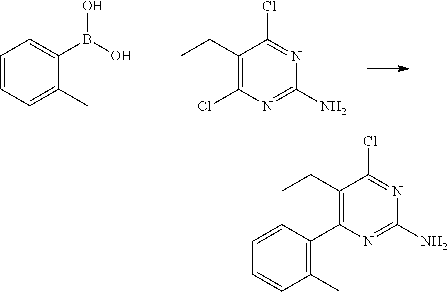

A heterogeneous solution of o-tolylboronic acid (approximately 566.4 mg, 4.166 mmol), 4,6-dichloro-5-ethyl-pyrimidin-2-amine (0.800 g, 4.166 mmol), bis(triphenylphosphine)palladium(II) dichloride (approximately 87.74 mg, 0.1250 mmol), and potassium carbonate (approximately 1.152 g, 8.332 mmol) in 1,4-dioxane (9.816 mL) was sealed in a pressure vessel and heated to 80° C. for 16 hours. The reaction mixture was diluted with dichloromethane and a saturated solution of aqueous ammonium chloride was added. The organic layer was removed, and the aqueous layer further extracted with dichloromethane (3×). The combined organics were dried over magnesium sulfate, filtered, and concentrated. The crude mixture was purified by flash column chromatography on silica gel (25% ethyl acetate in hexanes) to give 4-chloro-5-ethyl-6-(o-tolyl)pyrimidin-2-amine (0.72 g, 70%) as a yellow crystalline solid. ESI-MS m/z calc. 247.08763, found 248.2 (M+1)⁺; Retention time: 0.6 minutes; LC method D.

### Step 2: N-[4-chloro-5-ethyl-6-(o-tolyl)pyrimidin-2-yl]-1-methyl-pyrazole-4-sulfonamide

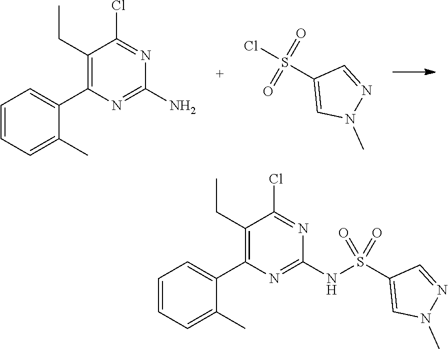

To a solution of 4-chloro-5-ethyl-6-(o-tolyl)pyrimidin-2-amine (720 mg, 2.906 mmol) in DMF (11.62 mL) at 0° C. was added NaH (approximately 278.9 mg, 11.62 mmol) and the reaction mixture was stirred at this temperature for 5 minutes, then removed from the cooling bath and stirred at room temperature for 10 minutes. The reaction mixture was cooled to 0° C. and 1-methylpyrazole-4-sulfonyl chloride (approximately 1.050 g, 5.812 mmol) was added dropwise as a solution in a small quantity of DMF. The reaction mixture was stirred at this temperature for 5 minutes, then removed from the cooling bath and stirred at room temperature for 12 minutes. The reaction mixture was cooled back to 0° C. and quenched with HCl (1.347 mL of 37% w/v, 13.67 mmol), then diluted with a solution of ethyl acetate/hexanes (1:1) and water. A saturated aqueous solution of sodium bicarbonate was added until the heterogeneous mixture was completely dissolved, and the acidic solution was neutralized. The organic layer was removed, and the aqueous layer was further extracted with ethyl acetate/hexanes (1:1, 5×). The combined organic extracts were dried with brine and magnesium sulfate. The solution was filtered, and the filtrate was concentrated in vacuo. The crude residue was purified by flash column chromatography on silica gel (5 to 40% ethyl acetate in hexanes). N-[4-chloro-5-ethyl-6-(o-tolyl)pyrimidin-2-yl]-1-methyl-pyrazole-4-sulfonamide (0.85 g, 67%) was isolated as a white solid. ESI-MS m/z calc. 391.08698, found 392.27 (M+1)⁺; Retention time: 0.65 minutes; LC method D.

### Step 3: N-[5-ethyl-4-(2-isopropylphenoxy)-6-(o-tolyl)pyrimidin-2-yl]-1-methyl-pyrazole-4-sulfonamide (Compound 440)

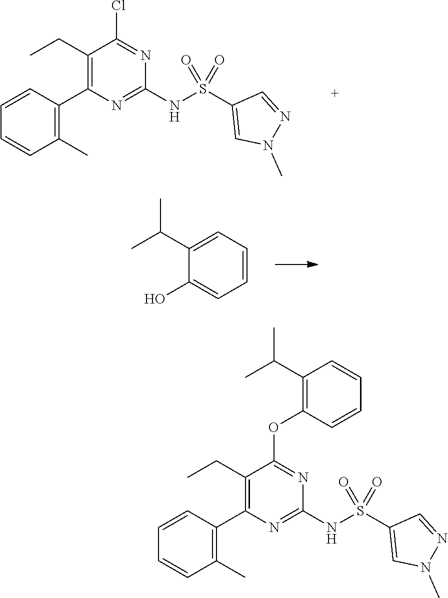

A heterogeneous solution of N-[4-chloro-5-ethyl-6-(o-tolyl)pyrimidin-2-yl]-1-methyl-pyrazole-4-sulfonamide (30 mg, 0.07656 mmol), 2-isopropylphenol (approximately 31.28 mg, 0.2297 mmol), and cesium carbonate (approximately 149.7 mg, 0.4594 mmol) in NMP (306.3 μL) were heated in a sealed vial at 110° C. for 16 hours. To the crude residue was added DMSO (0.60 mL) and the solution was separated by reverse-phase chromatography (acetonitrile in water with 0.1% hydrochloric acid). N-[5-ethyl-4-(2-isopropylphenoxy)-6-(o-tolyl)pyrimidin-2-yl]-1-methyl-pyrazole-4-sulfonamide was isolated as a white solid. ESI-MS m/z calc. 491.1991, found 492.45 (M+1)⁺; Retention time: 1.97 minutes; LC method A.

### Example 152: Preparation of Compound 441

### Step 1: 1-[4-(2-Chloro-3-hydroxy-phenyl)piperazin-1-yl]ethanone

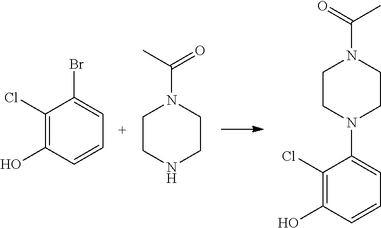

A heterogeneous mixture of 3-bromo-2-chloro-phenol (1.2 g, 5.784 mmol), 1-piperazin-1-ylethanone (6.9 g, 53.83 mmol), t-BuXPhos palladacycle Gen 1 (602 mg, 0.8767 mmol), and potassium t-butoxide (1.42 g, 12.65 mmol) in dioxane (35 mL) was sonicated for 15 minutes and heated in a sealed vessel at 50° C. for 16 hours. The reaction mixture was partitioned between DCM (50 mL) and 1 M HCl (50 mL). The organic layer was separated, and the aqueous layer was further extracted with DCM (2×). The combined organics were washed once with brine, dried using magnesium sulfate, filtered, and concentrated in vacuo. The resulting solid was triturated with dichloromethane. The solid was collected and further dried to give 1-[4-(2-chloro-3-hydroxy-phenyl)piperazin-1-yl]ethanone (810 mg, 55%). ESI-MS m/z calc. 254.0822, found 255.2 (M+1)⁺; Retention time: 0.34 minutes; LC method D.

### Step 2: N-[4-[3-(4-acetylpiperazin-1-yl)-2-chloro-phenoxy]-6-chloro-5-ethyl-pyrimidin-2-yl]-1-methyl-pyrazole-4-sulfonamide

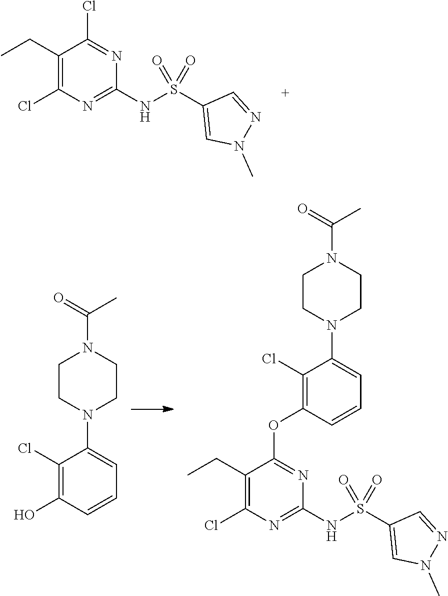 [SMILES: CCc1c(Cl)nc(NS(=O)(=O)c2cnn(C)c2)nc1Cl ; CC(=O)N1CCN(c2cccc(O)c2Cl)CC1 ; CCc1c(Cl)nc(NS(=O)(=O)c2cnn(C)c2)nc1Oc1cccc(N2CCN(C(C)=O)CC2)c1Cl]

A heterogeneous solution of N-(4,6-dichloro-5-ethyl-pyrimidin-2-yl)-1-methyl-pyrazole-4-sulfonamide (552 mg, 1.642 mmol), 1-[4-(2-chloro-3-hydroxy-phenyl)piperazin-1-yl]ethanone (416 mg, 1.633 mmol), and potassium carbonate (765 mg, 5.535 mmol) in NMP (2 mL) was heated in a sealed vial to 120° C. for 16 hours. The reaction was cooled and partitioned between dichloromethane (10 mL) and a 1 M HCl solution (10 mL). The organics were separated, washed with brine, dried over magnesium sulfate, filtered and evaporated. The crude material was purified by silica gel chromatography eluting with 0-10% methanol in dichloromethane to give N-[4-[3-(4-acetylpiperazin-1-yl)-2-chloro-phenoxy]-6-chloro-5-ethyl-pyrimidin-2-yl]-1-methyl-pyrazole-4-sulfonamide (791 mg, 87%) ESI-MS m/z calc. 553.10657, found 554.1 (M+1)⁺; Retention time: 0.63 minutes; LC method D.

### Step 3: N-[4-[3-(4-acetylpiperazin-1-yl)-2-chloro-phenoxy]-5-ethyl-6-(o-tolyl)pyrimidin-2-yl]-1-methyl-pyrazole-4-sulfonamide

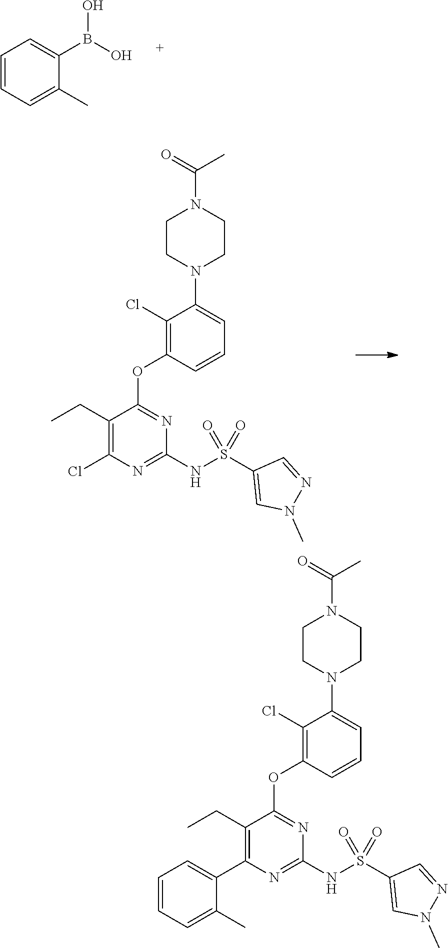

N-[4-[3-(4-acetylpiperazin-1-yl)-2-chloro-phenoxy]-6-chloro-5-ethyl-pyrimidin-2-yl]-1-methyl-pyrazole-4-sulfonamide (30 mg, 0.05411 mmol), o-tolylboronic acid (approximately 8.828 mg, 0.06493 mmol), tetrakis(triphenylphosphine)palladium (0) (approximately 6.253 mg, 0.005411 mmol), and 2 M aqueous potassium carbonate (approximately 108.2 μL of 2 M, 0.2164 mmol) were combined in dioxane and irradiated under microwave for 30 minutes at 120° C. The reaction mixture was filtered and purified by LC/MS utilizing a gradient of 1-99% acetonitrile in 5 mM aqueous HCl to yield N-[4-[3-(4-acetylpiperazin-1-yl)-2-chloro-phenoxy]-5-ethyl-6-(o-tolyl)pyrimidin-2-yl]-1-methyl-pyrazole-4-sulfonamide (12.5 mg, 36%). ESI-MS m/z calc. 609.1925, found 610.1 (M+1)⁺; Retention time: 1.71 minutes; LC method A.

### Example 153: Preparation of Compound 442

### Step 1: 4-(4-Methylpiperazin-1-yl)-3-(trifluoromethyl)phenol

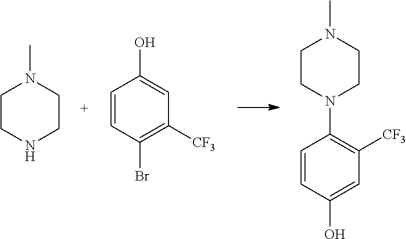

A dioxane (3 mL) mixture of 1-methylpiperazine (123.7 mg, 1.235 mmol), [2-(2-aminoethyl)phenyl]-chloro-palladium;di tert-butyl-[2-(2,4,6-triisopropylphenyl)phenyl]phosphane (41 mg, 0.05971 mmol), 4-bromo-3-(trifluoromethyl)phenol (0.1438 g, 0.5967 mmol), and sodium tert-butoxide (145.2 mg, 1.511 mmol) was sparged with nitrogen under sonication for 5 minutes and then stirred at 30° C. for 2 hours. The mixture was poured into aqueous sat. ammonium chloride (20 mL) and the product was extracted with dichloromethane (20 mL×2). The organic layer was dried over magnesium sulfate, filtered, and concentrated in vacuo. The resulting residues were dissolved in 1.8 mL (1:1) DMSO/MeOH, and purified by reverse phase chromatography using a 15 minutes gradient of 20% MeCN in water to 80% MeCN (HCl modifier) to give 4-(4-methylpiperazin-1-yl)-3-(trifluoromethyl)phenol (hydrochloride salt) (53 mg, 30%) ESI-MS m/z calc. 260.11365, found 261.33 (M+1)⁺; Retention time: 0.74 minutes. LC method A.

### Step 2: N-[4-(2,6-Dimethylphenyl)-6-[4-(4-methylpiperazin-1-yl)-3-(trifluoromethyl)phenoxy]pyrimidin-2-yl]-1-methyl-pyrazole-4-sulfonamide

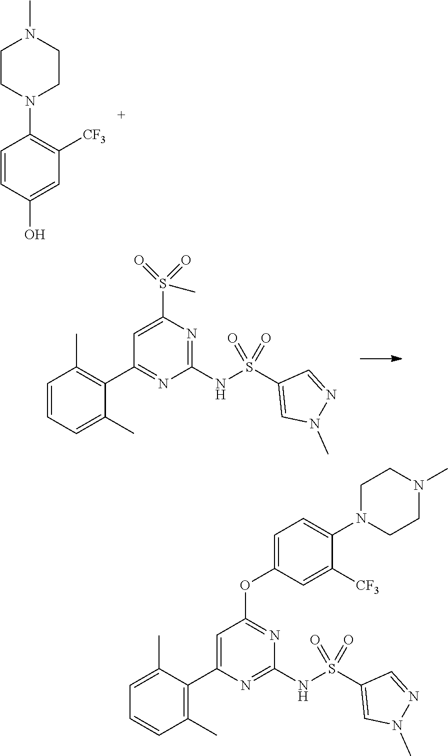

A NMP (500 μL) solution of 4-(4-methylpiperazin-1-yl)-3-(trifluoromethyl)phenol (hydrochloride salt) (approximately 21.12 mg, 0.07118 mmol), N-[4-(2,6-dimethylphenyl)-6-methylsulfonyl-pyrimidin-2-yl]-1-methyl-pyrazole-4-sulfonamide (15 mg, 0.03559 mmol), and Cs₂CO₃ (approximately 46.40 mg, 0.1424 mmol) was stirred at 110° C. for 3 hours and then cooled to room temperature. The solution was filtered, and the resulting residue dissolved in 0.8 mL MeOH, and purified by reverse phase chromatography using a 15 minutes gradient of 20% MeCN in water to 80% MeCN (HCl modifier) to give the title compound. N-[4-(2,6-dimethylphenyl)-6-[4-(4-methylpiperazin-1-yl)-3-(trifluoromethyl)phenoxy]pyrimidin-2-yl]-1 -methyl-pyrazole-4-sulfonamide (12.7 mg, 84%). ESI-MS m/z calc. 601.2083, found 602.57 (M+1)⁺; Retention time: 1.38 minutes; LC method A.

### Example 154: Preparation of Compound 443

### Step 1: 4-(4-Methylpiperazin-1-yl)-2-(trifluoromethoxy)phenol

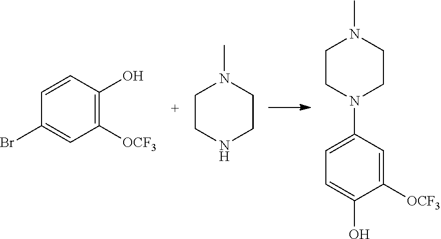

A dioxane (1 mL) mixture of 4-bromo-2-(trifluoromethoxy)phenol (49.7 mg, 0.1934 mmol), 1-methylpiperazine (70 μL, 0.6304 mmol), chloro(2-di-t-butylphosphino-2′, 4′, 6′-tri-i-propyl-1,1′-biphenyl)[2-(2-aminoethyl)phenyl] palladium(II)[t-BuXPhos Palladacycle Gen. 1](23.3 mg, 0.03393 mmol), and sodium tert-butoxide (83.1 mg, 0.8647 mmol) was sparged with nitrogen for 5 minutes and then stirred at 30° C. for 2 hours. The solution was filtered, and the filtrate dissolved in 0.8 mL MeOH, and purified by reverse phase chromatography using a 15 minutes gradient of 20% MeCN in water to 80% MeCN (HCl modifier) to give 4-(4-methylpiperazin-1-yl)-2-(trifluoromethoxy)phenol.

### Step 2: N-[4-(2,6-Dimethylphenyl)-6-[4-(4-methylpiperazin-1-yl)-2-trifluoromethoxy)phenoxy]pyrimidin-2-yl]-1-methyl-pyrazole-4-sulfonamide

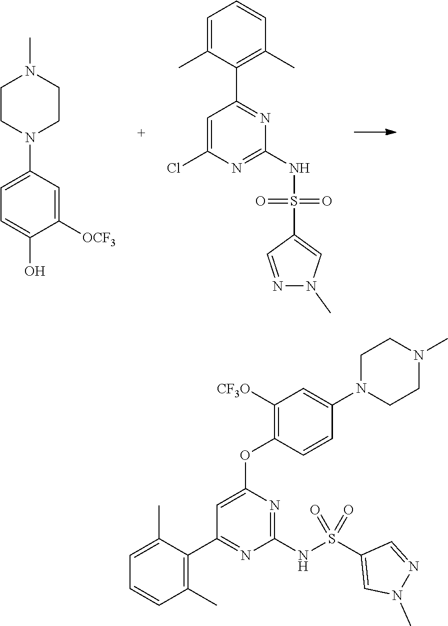

A NMP (0.6 mL) mixture of 4-(4-methylpiperazin-1-yl)-2-(trifluoromethoxy)phenol (12.9 mg, 0.04125 mmol), N-[4-chloro-6-(2,6-dimethylphenyl)pyrimidin-2-yl]-1-methyl-pyrazole-4-sulfonamide (11.6 mg, 0.03070 mmol), and Cs₂CO₃ (54.3 mg, 0.1667 mmol) was stirred at 110° C. for 16 hours and then cooled to room temperature. The solution was filtered and the filtrate was diluted in 0.8 mL MeOH, and purified by reverse phase chromatography using a 15 minutes 1-99% gradient of MeCN in water (HCl modifier) to give N-[4-(2,6-dimethylphenyl)-6-[4-(4-methylpiperazin-1-yl)-2-(trifluoromethoxy)phenoxy]pyrimidin-2-yl]-1 -methyl-pyrazole-4-sulfonamide (hydrochloride salt) (4.6 mg, 23%). ESI-MS m/z calc. 617.2032, found 618.2 (M+1)⁺; Retention time: 1.37 minutes; LC method A.

### Example 155: Preparation of Compound 444

### Step 1: N-[4-[3,5-Dimethyl-4-(4-methylpiperazin-1-yl)phenoxy]-6-(2,6-dimethylphenyl)pyrimidin-2-yl]-1-methyl-pyrazole-4-sulfonamide

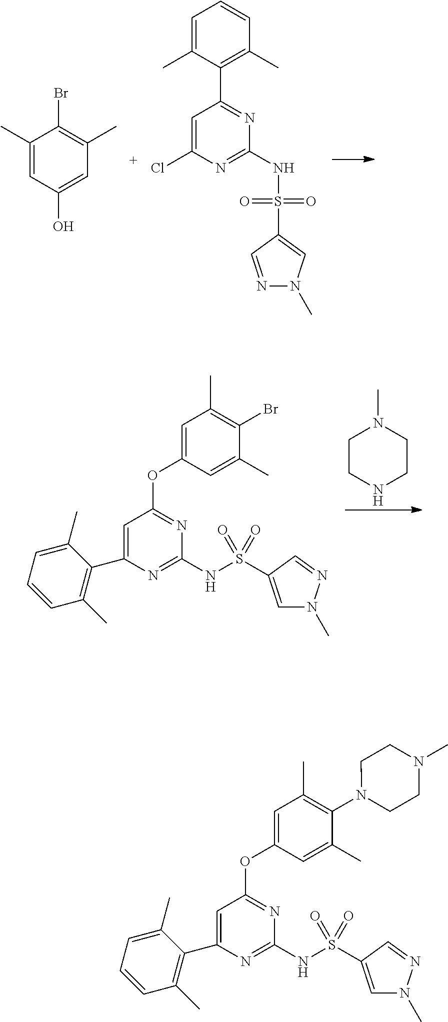

A NMP (1 mL) mixture of 4-bromo-3,5-dimethyl-phenol (85.3 mg, 0.4243 mmol), N-[4-chloro-6-(2,6-dimethylphenyl)pyrimidin-2-yl]-1-methyl-pyrazole-4-sulfonamide (48.2 mg, 0.1276 mmol), and Cs₂CO₃ (166.6 mg, 0.5113 mmol) was stirred at 110° C. for 16 hours and then cooled to room temperature. The solution was filtered and the filtrate dissolved in 0.8 mL MeOH, and purified by reverse phase chromatography using a 15 minutes 1-99% gradient of MeCN in water (HCl modifier) to give N-[4-(4-bromo-3,5-dimethyl-phenoxy)-6-(2,6-dimethylphenyl)pyrimidin-2-yl]-1-methyl-pyrazole-4-sulfonamide (48.7 mg, 70%). ESI-MS m z calc. 541.0783, found 542.41 (M+1)⁺; Retention time: 2.19 minutes.

N-[4-(4-bromo-3,5-dimethyl-phenoxy)-6-(2,6-dimethylphenyl)pyrimidin-2-yl]-1 -methyl-pyrazole-4-sulfonamide (12.2 mg, 0.02249 mmol) from above is charged to a dioxane (0.5 mL) mixture of sodium tert-butoxide (approximately 5.846 mg, 0.06083 mmol), 1-methylpiperazine (30 μL), and [2-(2-aminophenyl)phenyl]-chloro-palladium;dicyclohexyl-[2-(2,4,6-triisopropylphenyl)phenyl]phosphane (approximately 11.32 mg, 0.01439 mmol) and the mixture was sparged with nitrogen for 1 minute and then stirred at 50° C. for 16 hours. The solution was filtered and the filtrate diluted with 0.8 mL MeOH, and purified by reverse phase chromatography using a 15 minutes 20-80% gradient of MeCN in water (HCl modifier) to give N-[4-[3, 5-dimethyl-4-(4-methylpiperazin-1-yl)phenoxy]-6-(2,6-dimethylphenyl)pyrimidin-2-yl]-1-methyl-pyrazole-4-sulfonamide (hydrochloride salt) (1.6 mg, 2%) ESI-MS m/z calc. 561.2522, found 562.55 (M+1)⁺; Retention time: 1.39 minutes; LC method A.

### Example 156: Preparation of Compound 445

### Step 1: 3-Chloro-4-(4-methylpiperazin-1-yl)phenol

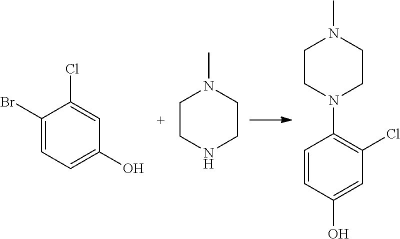

A dioxane (11 mL) solution of 4-bromo-3-chloro-phenol (455.8 mg, 2.197 mmol), [2-(2-aminoethyl)phenyl]-chloro-palladium;ditert-butyl-[2-(2,4,6-triisopropylphenyl)phenyl]phosphane (XPhos Pd G1)(184.6 mg, 0.2688 mmol), 1-methylpiperazine (approximately 2.201 g, 2.440 mL, 21.97 mmol), and sodium tert-butoxide (approximately 527.9 mg, 5.493 mmol) ) was sparged with nitrogen at room temperature for 15 minutes and then heated at 50° C. for 16 hours. The reaction was then cooled to room temperature and poured into a saturated aqueous solution of ammonium chloride (38 mL) and dichloromethane (80 mL). The two layers were separated, and the organic layer was dried with anhydrous sodium sulfate, filtered, concentrated in vacuo and purified by flash column chromatography (12 g silica) using a 15 minute gradient of 0%-10% MeOH in DCM to give 3-chloro-4-(4-methylpiperazin-1-yl)phenol (256.8 mg, 52%). ¹H NMR (400 MHz, DMSO-d₆) δ 9.47 (s, 1H), 7.00 (d, J=8.7 Hz, 1H), 6.80 (d, J=2.8 Hz, 1H), 6.68 (dd, J=8.7, 2.8 Hz, 1H), 2.83 (t, J=4.9 Hz, 4H), 2.44 (s, 4H), 2.21 (s, 3H). ESI-MS m/z calc. 226.0873, found 227.29 (M+1)⁺; Retention time: 0.57 minutes; LC method A.

### Step 2: N-[4-[3-Chloro-4-(4-methylpiperazin-1-yl)phenoxy]-6-(2,6-dimethylphenyl)pyrimidin-2-yl]-1-methyl-pyrazole-4-sulfonamide

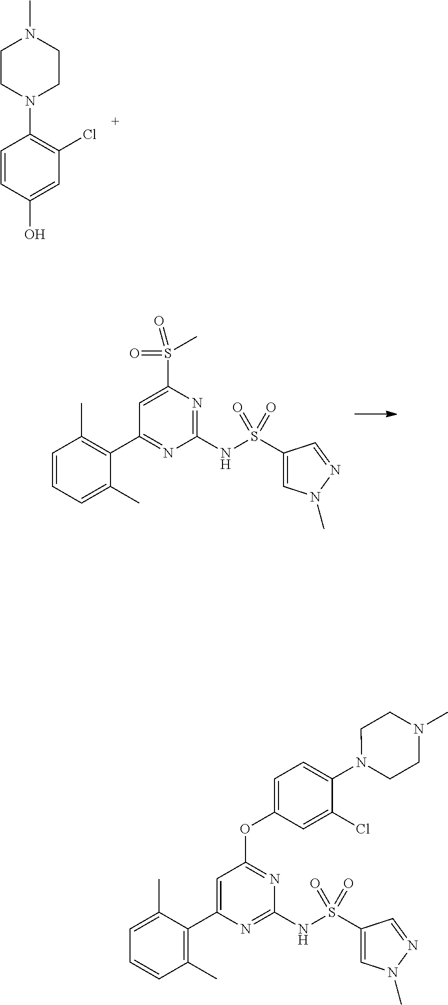 [SMILES: CN1CCN(c2ccc(O)cc2Cl)CC1 ; Cc1cccc(C)c1-c1cc(S(C)(=O)=O)nc(NS(=O)(=O)c2cnn(C)c2)n1 ; Cc1cccc(C)c1-c1cc(Oc2ccc(N3CCN(C)CC3)c(Cl)c2)nc(NS(=O)(=O)c2cnn(C)c2)n1]

A NMP (0.6 mL) mixture of 3-chloro-4-(4-methylpiperazin-1-yl)phenol (approximately 17.75 mg, 0.07830 mmol), Cs₂CO₃ (approximately 34.02 mg, 0.1044 mmol)N-[4-(2,6-dimethylphenyl)-6-methylsulfonyl-pyrimidin-2-yl]-1-methyl-pyrazole-4-sulfonamide (11 mg, 0.02610 mmol) was heated to 110° C. for 2 hours and then cooled to room temperature. The solutions was filtered and the resulting residue dissolved in 0.8 mL MeOH, and purified by reverse phase chromatography using a 15 minutes 20-80% gradient of MeCN in water (HCl modifier) to give the title compound N-[4-[3-chloro-4-(4-methylpiperazin-1-yl)phenoxy]-6-(2,6-dimethylphenyl)pyrimidin-2-yl]-1-methyl-pyrazole-4-sulfonamide (9.7 mg, 68%). ESI-MS m z calc. 567.18195, found 568.53 (M+1)⁺; Retention time: 1.3 minutes; LC method A. ¹H NMR (400 MHz, DMSO-d₆) δ 10.48 (s, 1H), 7.77 (s, 1H), 7.58 (d, J=2.6 Hz, 1H), 7.41-7.29 (m, 2H), 7.29-7.20 (m, 1H), 7.18-7.11 (m, 3H), 6.66 (s, 1H), 3.55-3.41 (m, 4H), 3.34-3.04 (m, 4H), 2.86 (d, J=4.6 Hz, 3H), 2.04 (s, 6H). (3H likely overlapped under the water signal).

### Example 157: Preparation of Compound 446

### Step 1: 4-Bromo-2-(2-methylprop-1-enyl)phenol

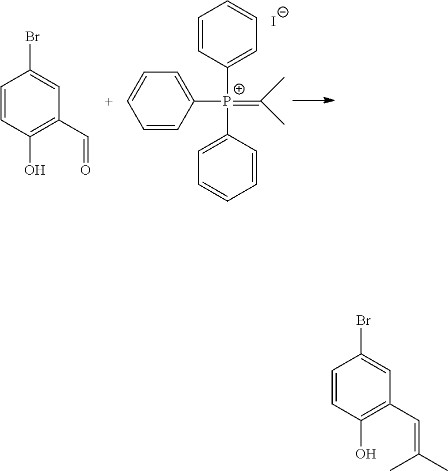

A THE (10 mL) mixture of isopropyl(triphenyl)phosphonium iodide (2.9671 g, 6.864 mmol) at 0° C. was treated with potassium tert-butoxide (773.5 mg, 6.893 mmol) and then warmed to room temperature and stirred for 1.5 hours. The reaction mixture was cooled to 0° C. and 5-bromo-2-hydroxy-benzaldehyde (606.0 mg, 3.015 mmol) was added and the reaction was warmed to room temperature and stirred for 1 hour and then quenched with HCl (9 mL of 1 M, 9.000 mmol) and diluted with diethyl ether (20 ml). The organic layer was separated and washed with water (5 mL) and then brine (5 mL) and dried over anhydrous sodium sulfate, filtered, and concentrated in vacuo to give 4-bromo-2-(2-methylprop-1-enyl)phenol (719.3 mg, 105%). ESI-MS m/z calc. 225.99933, Retention time: 0.71 minutes. Mass not observed.

### Step 2: 4-(4-Methylpiperazin-1-yl)-2-(2-methylprop-1-enyl)phenol

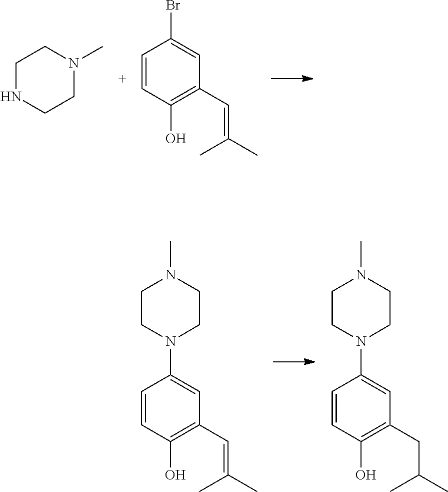

A dioxane (4 mL) mixture of 1-methylpiperazine (120.1 mg, 1.199 mmol), chloro(2-di-t-butylphosphino-2′, 4′, 6′-tri-i-propyl-1,1′-biphenyl)[2-(2-aminoethyl)phenyl] palladium(II) (57.6 mg, 0.08388 mmol), potassium tert-butoxide (298.5 mg, 2.660 mmol), and 4-bromo-2-(2-methylprop-1-enyl)phenol (90.1 mg, 0.3967 mmol) was stirred at room temperature for 15 minutes and then quenched with AcOH (50 μL, 0.8792 mmol). The solution was filtered and the filtrate diluted with 0.8 mL MeOH, and purified by reverse phase chromatography using a 15 minutes gradient of 1% MeCN in water to 50% MeCN (HCl modifier) to give 4-(4-methylpiperazin-1-yl)-2-(2-methylprop-1-enyl)phenol (hydrochloride salt) (20.5 mg, 18%). ESI-MS m/z calc. 246.17322, found 247.2 (M+1)⁺; Retention time: 0.88 minutes. LC method A.

The product from above was taken up in EtOH (1 mL) and treated with Pd on C, wet, Degussa (20.2 mg, 0.01898 mmol). The system was evacuated and backfilled with nitrogen (3×) and then stirred under an atmosphere of hydrogen (balloon) for 2 hours and then filtered over a bed of Celite. The reaction mixture was concentrated in vacuo to give 2-isobutyl-4-(4-methylpiperazin-1-yl)phenol (hydrochloride salt) (19.7 mg, 17%). ESI-MS m/z calc. 248.18886, found 249.2 (M+1)⁺; Retention time: 0.44 minutes; LC method D.

### Step 3: N-[4-(2,6-Dimethylphenyl)-6-[2-isobutyl-4-(4-methylpiperazin-1-yl)phenoxy]pyrimidin-2-yl]-1-methyl-pyrazole-4-sulfonamide

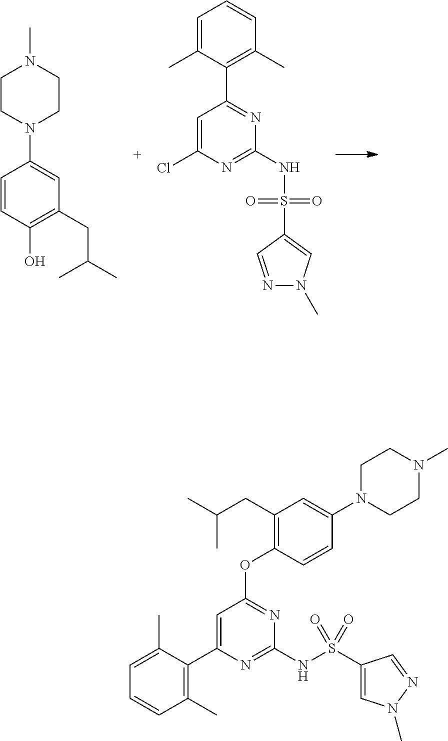

A NMP (0.5 mL) mixture of 2-isobutyl-4-(4-methylpiperazin-1-yl)phenol (hydrochloride salt) (56.7 mg, 0.1991 mmol), N-[4-chloro-6-(2,6-dimethylphenyl)pyrimidin-2-yl]-1-methyl-pyrazole-4-sulfonamide (50.1 mg, 0.1326 mmol), and Cs₂CO₃ (169.2 mg, 0.5193 mmol) was stirred at 140° C. for 16 hours. The solution was filtered and the filtrate diluted in 0.8 mL MeOH, and purified by reverse phase chromatography using a 15 minutes gradient of 1% MeCN in water to 99% MeCN with ammonium formate modifier to give N-[4-(2,6-dimethylphenyl)-6-[2-isobutyl-4-(4-methylpiperazin-1-yl)phenoxy]pyrimidin-2-yl]-1-methyl-pyrazole-4-sulfonamide (formic acid salt) (3.8 mg, 5%). ESI-MS m/z calc. 589.2835, found 590.4 (M+1)⁺; Retention time: 1.36 minutes; LC method A.

### Example 158: Preparation of Compound 447

### Step 1: N-[4-[3-Chloro-5-methyl-4-(4-methylpiperazin-1-yl)phenoxy]-6-(2,6-dimethylphenyl)pyrimidin-2-yl]-1-methyl-pyrazole-4-sulfonamide

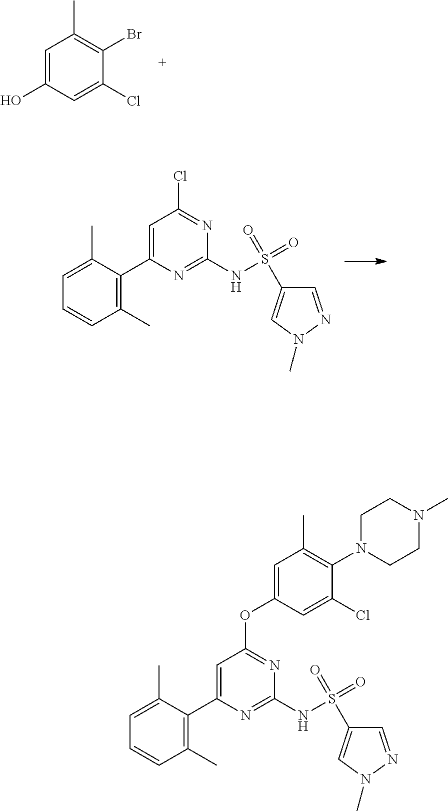

A NMP (1 mL) mixture of 4-bromo-3-chloro-5-methyl-phenol (87.2 mg, 0.3937 mmol), N-[4-chloro-6-(2,6-dimethylphenyl)pyrimidin-2-yl]-1-methyl-pyrazole-4-sulfonamide (47.8 mg, 0.1265 mmol), and Cs₂CO₃ (166.2 mg, 0.5101 mmol) was stirred at 120° C. for 16 hours and then cooled to room temperature. The solution was filtered and the filtrate dissolved in 0.8 mL MeOH, and purified by reverse phase chromatography using a 15 minutes gradient of 10% MeCN in water to 99% MeCN (HCl modifier) to give N-[4-(4-bromo-3-chloro-5-methyl-phenoxy)-6-(2,6-dimethylphenyl)pyrimidin-2-yl]-1-methyl-pyrazole-4-sulfonamide (56.2 mg, 79%). ESI-MS m/z calc. 561.0237, found 562.34 (M+1)⁺; Retention time: 2.17 minutes. LC method A.

N-[4-(4-bromo-3-chloro-5-methyl-phenoxy)-6-(2,6-dimethylphenyl)pyrimidin-2-yl]-1-methyl-pyrazole-4-sulfonamide (26.2 mg, 0.04655 mmol) isolated from above was taken up in a dioxane (0.5 mL) mixture of 1-methylpiperazine (approximately 12.67 mg, 0.1265 mmol), chloro(2-di-t-butylphosphino-2′, 4′, 6′-tri-i-propyl-1,1′-biphenyl)[2-(2-aminoethyl)phenyl]palladium(II) [t-BuXPhos Palladacycle Gen. 1] (14.8 mg, 0.02155 mmol), and sodium tert-butoxide (13.43 mg, 0.1397 mmol) and the mixture was sparged with nitrogen for 1 minute and then stirred at 50° C. for 16 hours. The solution was filtered and the filtrate was diluted with 0.8 mL DMSO, and purified by reverse phase chromatography using a 15 minutes gradient of 20% MeCN in water to 80% MeCN (HCl modifier) to give N-[4-[3-chloro-5-methyl-4-(4-methylpiperazin-1-yl)phenoxy]-6-(2,6-dimethylphenyl)pyrimidin-2-yl]-1-methyl-pyrazole-4-sulfonamide (hydrochloride salt) (1.1 mg, 1%) ESI-MS m/z calc. 581.1976, found 582.2 (M+1)⁺; Retention time: 1.33 minutes. LC method A.

### Example 159: Preparation of Compound 448

### Step 1: N-[4-(4-bromo-3,5-dichloro-phenoxy)-6-(2,6-dimethylphenyl)pyrimidin-2-yl]-1-methyl-pyrazole-4-sulfonamide

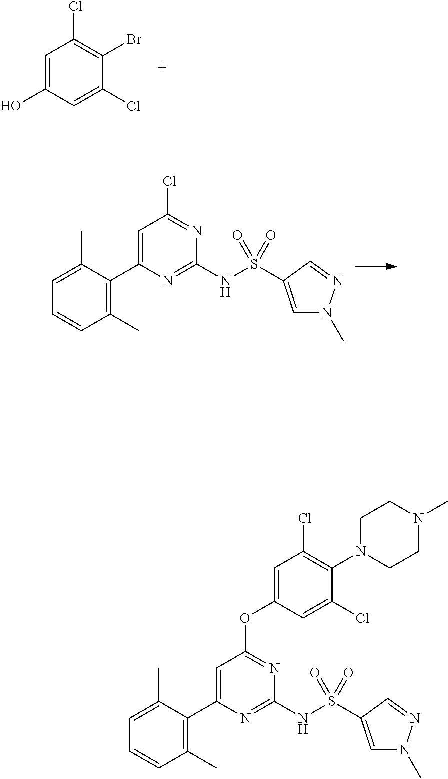 [SMILES: Oc1cc(Cl)c(Br)c(Cl)c1 ; Cc1cccc(C)c1-c1cc(Cl)nc(NS(=O)(=O)c2cnn(C)c2)n1 ; Cc1cccc(C)c1-c1cc(Oc2cc(Cl)c(N3CCN(C)CC3)c(Cl)c2)nc(NS(=O)(=O)c2cnn(C)c2)n1]

A NMP (0.5 mL) mixture of 4-bromo-3,5-dichloro-phenol (approximately 7.673 mg, 0.03172 mmol), N-[4-chloro-6-(2,6-dimethylphenyl)pyrimidin-2-yl]-1-methyl-pyrazole-4-sulfonamide (approximately 11.99 mg, 0.03172 mmol), and Cs₂CO₃ (10.1 mg, 0.03100 mmol) was stirred at 110° C. for 16 hours and then cooled to room temperature. The solution was filtered and the filtrate diluted with 0.8 mL DMSO, and purified by reverse phase chromatography using a 15 minutes gradient of 1% MeCN in water to 99% MeCN (HCl modifier) to give N-[4-(4-bromo-3,5-dichloro-phenoxy)-6-(2,6-dimethylphenyl)pyrimidin-2-yl]-1-methyl-pyrazole-4-sulfonamide (16.1 mg, 87%). ESI-MS m/z calc. 580.96906, found 582.26 (M+1)⁺; Retention time: 2.08 minutes. LC method A.

The material above was taken up in dioxane (0.7 mL) and was combined with 1-methylpiperazine (4.15 mg, 0.04143 mmol), Chloro(2-di-t-butylphosphino-2′, 4′, 6′-tri-i-propyl-1,1′-biphenyl)[2-(2-aminoethyl)phenyl] palladium(II) (t-BuXPhos Palladacycle Gen. 1) (2.1 mg, 0.003225 mmol), sodium tert-butoxide (12.2 mg, 0.1269 mmol) and the reaction mixture was sparged with nitrogen for 1 minute under sonication and then stirred at 80° C. for 16 hours. The solution was filtered and the filtrate dissolved in 0.8 mL MeOH, and purified by reverse phase chromatography using a 15 min gradient of 20% MeCN in water to 80% MeCN (HCl modifier) to give N-[4-[3,5-dichloro-4-(4-methylpiperazin-1-yl)phenoxy]-6-(2,6-dimethylphenyl)pyrimidin-2-yl]-1-methyl-pyrazole-4-sulfonamide (hydrochloride salt) (2.1 mg, 10%) ESI-MS m/z calc. 601.14294, found 602.1 (M+1)⁺; Retention time: 1.34 minutes. LC method A.

### Example 160: Preparation of Compound 449

### Step 1: 5-Hydroxy-2-(4-methylpiperazin-1-yl)benzonitrile

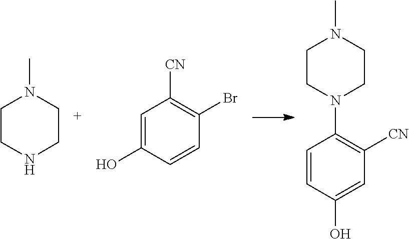

A dioxane (4.4 mL) mixture of 1-methylpiperazine (0.35 mL, 3.2 mmol), sodium tert-butoxide (225.5 mg, 2.346 mmol), [2-(2-aminoethyl)phenyl]-chloro-palladium;ditert-butyl-[2-(2,4,6-triisopropylphenyl)phenyl]phosphane (XPhos Pd G1)(170.9 mg, 0.2489 mmol), and 2-bromo-5-hydroxy-benzonitrile (209 mg, 1.055 mmol) was sparged with nitrogen under sonication for 5 minutes and then stirred at 30° C. for 2 hours. The mixture was poured into aqueous saturated ammonium chloride (20 mL) and the product was extracted with dichloromethane (20 mL×2). The organic layer was dried with magnesium sulfate, filtered, and concentrated in vacuo. The resulting residue was dissolved in 1.8 mL (1:1) DMSO/MeOH, and purified by reverse phase chromatography using a 15 minutes gradient of 1% MeCN in water to 50% MeCN (HCl modifier) to give 5-hydroxy-2-(4-methylpiperazin-1-yl)benzonitrile (hydrochloride salt) (57 mg, 21%). ESI-MS m/z calc. 217.1215, found 218.29 (M+1)⁺; Retention time: 0.48 minutes. LC method A.

### Step 2: N-[4-[3-cyano-4-(4-methylpiperazin-1-yl)phenoxy]-6-(2,6-dimethylphenyl)pyrimidin-2-yl]-1-methyl-pyrazole-4-sulfonamide

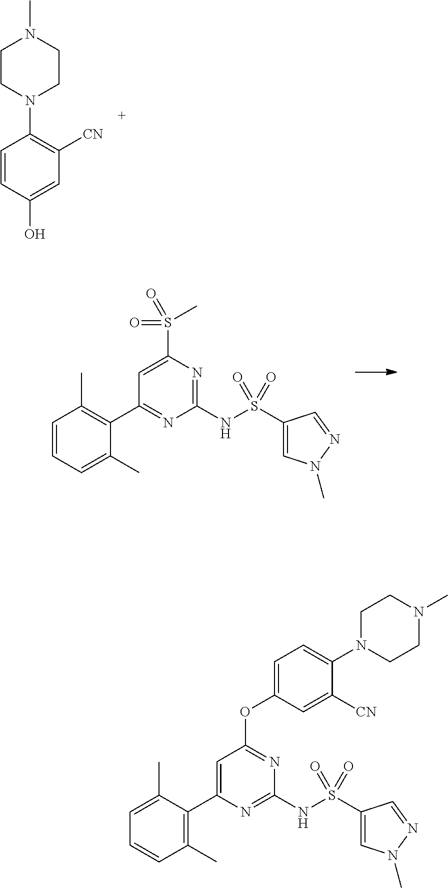 [SMILES: CN1CCN(c2ccc(O)cc2C#N)CC1 ; Cc1cccc(C)c1-c1cc(S(C)(=O)=O)nc(NS(=O)(=O)c2cnn(C)c2)n1 ; Cc1cccc(C)c1-c1cc(Oc2ccc(N3CCN(C)CC3)c(C#N)c2)nc(NS(=O)(=O)c2cnn(C)c2)n1]

A NMP (500 μL) solution of 5-hydroxy-2-(4-methylpiperazin-1-yl)benzonitrile (hydrochloride salt) (approximately 18.06 mg, 0.07118 mmol), N-[4-(2,6-dimethylphenyl)-6-methylsulfonyl-pyrimidin-2-yl]-1-methyl-pyrazole-4-sulfonamide (15 mg, 0.03559 mmol), and Cs₂CO₃ (approximately 46.40 mg, 0.1424 mmol) was stirred at 110° C. for 3 hours and then cooled to room temperature. The solution was filtered, and the resulting residue dissolved in 0.8 mL MeOH, and purified by reverse phase chromatography using a 15 minutes gradient of 20% MeCN in water to 80% MeCN (HCl modifier) to give the title compound. N-[4-[3-cyano-4-(4-methylpiperazin-1-yl)phenoxy]-6-(2,6-dimethylphenyl)pyrimidin-2-yl]-1-methyl-pyrazole-4-sulfonamide (9.6 mg, 68%). ESI-MS m/z calc. 558.2162, found 559.57 (M+1)⁺; Retention time: 1.22 minutes; LC method A. ¹H NMR (400 MHz, DMSO-d₆) δ 7.89 (d, J=2.8 Hz, 1H), 7.83 (s, 1H), 7.63 (dd, J=8.9, 2.9 Hz, 1H), 7.40 (d, J=9.0 Hz, 1H), 7.28-7.22 (m, 1H), 7.18-7.08 (m, 3H), 6.70 (s, 1H), 3.78 (s, 3H), 3.68-3.51 (m, 4H), 3.34-3.18 (m, 4H), 2.86 (dd, J=4.8, 2.1 Hz, 3H), 2.04 (s, 6H).

### Example 161: Preparation of Compound 450

### Step 1: 4-(4-Methylpiperazin-1-yl)phenol

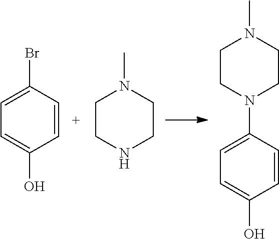

4-Bromophenol (40 g, 231.2 mmol) and 1-methylpiperazine (approximately 69.47 g, 693.6 mmol) were dissolved in dioxane (400.0 mL) and purged with nitrogen. Sodium tert-butoxide (approximately 66.66 g, 693.6 mmol) and tBuXPhos Pd G1 (approximately 3.011 g, 4.624 mmol) was added while purging with nitrogen and the brown suspension was then heated to 95° C. under nitrogen for 18 hours. The reaction mixture was cooled, neutralized by adding acetic acid (approximately 41.65 g, 39.44 mL, 693.6 mmol) and most of the solvent was removed under reduced pressure. The residue was treated with ethyl acetate (400 mL) and saturated sodium bicarbonate (500 mL) and charcoal. The mixture was stirred for 1 hour, filtered over Celite and extracted. The organic phase was washed once with sodium bicarbonate (500 mL) and the aqueous phases were back extracted once with ethyl acetate (300 mL). The combined organic phases were dried over magnesium sulfate, treated with charcoal and filtered. The crude (˜37 g deep brown purple mass) was purified by chromatography over silica gel (750 g, solid load with SiO₂) with a linear gradient of dichloromethane to 10% methanol to give 4-(4-methylpiperazin-1-yl)phenol (14.3 g, 32%) as a brown purple solid. ¹H NMR (400 MHz, DMSO-d₆) δ 8.80 (s, 1H), 6.81-6.70 (m, 2H), 6.70-6.59 (m, 2H), 3.00-2.86 (m, 4H), 2.46-2.37 (m, 4H), 2.20 (s, 3H). ESI-MS m/z calc. 192.12627, found 193.0 (M+1)⁺; Retention time: 0.39 minutes ((LC method J).

### Step 2: N-[4-(2,6-dimethylphenyl)-6-[4-(4-methylpiperazin-1-yl)phenoxy]pyrimidin-2-yl]-1-methyl-pyrazole-4-sulfonamide

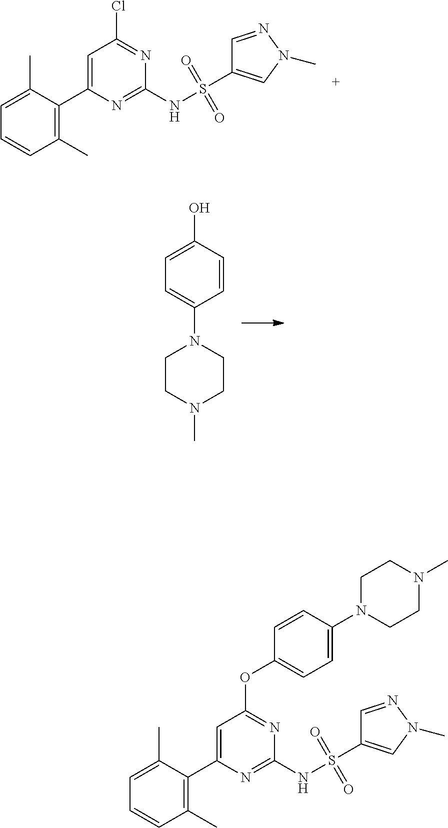 [SMILES: Cc1cccc(C)c1-c1cc(Cl)nc(NS(=O)(=O)c2cnn(C)c2)n1 ; CN1CCN(c2ccc(O)cc2)CC1 ; Cc1cccc(C)c1-c1cc(Oc2ccc(N3CCN(C)CC3)cc2)nc(NS(=O)(=O)c2cnn(C)c2)n1]

N-[4-chloro-6-(2,6-dimethylphenyl)pyrimidin-2-yl]-1-methyl-pyrazole-4-sulfonamide (22.6 g, 59.81 mmol) and 4-(4-methylpiperazin-1-yl)phenol (11.5 g, 59.8 mmol) were dissolved in NMP (225 mL) and treated with cesium fluoride (9.1 g, 60. mmol) followed by cesium carbonate (39.0 g, 120. mmol). The suspension was cycled 3 times with vacuum/nitrogen and heated at 117.5° C. for 38 hours. The reaction mixture was added to a stirred solution of acetic acid (21.6 g, 360. mmol) in water (1.13 L) (foaming, cloudy yellow solution with foam on top), stirred for 0.5 hour and washed twice with ethyl acetate (610 mL) and ethyl acetate (295 mL). The organic phases were washed once with a solution of acetic acid (6 mL, 100 mmol) in water (295 mL). The combined aqueous phases were treated with MP-TMT Pd— scavenger ((Biotage Part #801472, Lot #12170NJ, -15 g) and charcoal and stirred at room temperature for 1 hour. The mixture was filtered over celite (pale yellow clear solution, pH 5.2) and slowly treated with solid sodium bicarbonate (18.0 g, 214 mmol) (foaming, becomes cloudy) and after stirring for ˜5 minutes a fine cream suspension was obtained (pH 6.5). The cream suspension is stirred at room temperature for 3.5 hours, filtered and washed with water. The wet solid was then suspended in water (680 mL) heated to 80° C. and left to cool to room temperature under stirring for 2 hours. The solid was collected by filtration, washed with water and dried in a drying cabinet under vacuum at 45° C. with a nitrogen bleed till weight constant to give N-[4-(2,6-dimethylphenyl)-6-[4-(4-methylpiperazin-1-yl)phenoxy]pyrimidin-2-yl]-1 -methyl-pyrazole-4-sulfonamide (23.5 g, 72%) as an off white solid. ¹H NMR (400 MHz, DMSO-d₆) δ 7.60 (s, 1H), 7.35 (s, 1H), 7.28-7.19 (m, 1H), 7.20-7.09 (m, 4H), 7.09-6.99 (m, 2H), 6.45 (s, 1H), 3.73 (s, 3H), 3.16 (s, 4H), 2.54 (t, J=5.0 Hz, 4H), 2.27 (s, 3H), 2.03 (s, 6H). ESI-MS m/z calc. 533.2209, found 534.0 (M+1)⁺; Retention time: 1.17 minutes. LC method A

### Example 162: Preparation of Compound 451

### Step 1: N-[4-(2,6-Dimethylphenyl)-6-(4-piperazin-1-ylphenoxy)pyrimidin-2-yl]-1 -methyl-pyrazole-4-sulfonamide

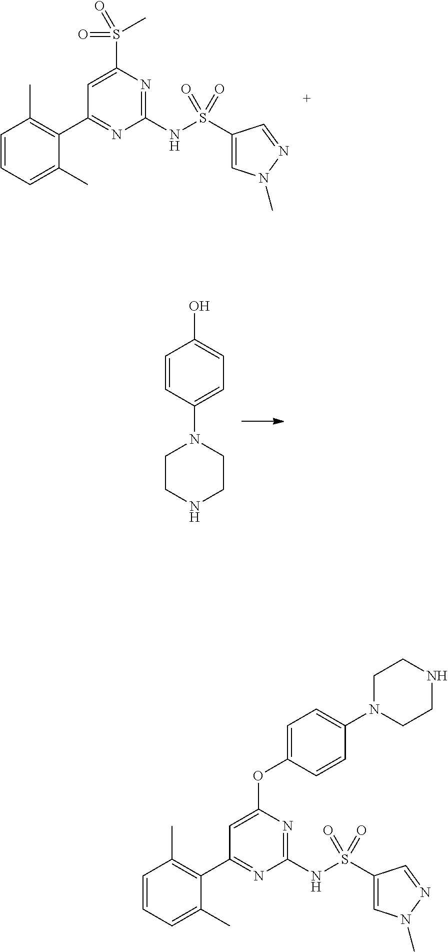 [SMILES: Cc1cccc(C)c1-c1cc(S(C)(=O)=O)nc(NS(=O)(=O)c2cnn(C)c2)n1 ; Oc1ccc(N2CCNCC2)cc1 ; Cc1cccc(C)c1-c1cc(Oc2ccc(N3CCNCC3)cc2)nc(NS(=O)(=O)c2cnn(C)c2)n1]

To a solution of N-[4-(2,6-dimethylphenyl)-6-methylsulfonyl-pyrimidin-2-yl]-1 -methyl-pyrazole-4-sulfonamide (50 mg, 0.1186 mmol) and 4-piperazin-1-ylphenol (74 mg, 0.4152 mmol) in NMP (500.0 μL) was added Cs₂CO₃ (165 mg, 0.5064 mmol) and the reaction mixture stirred at 80° C. for 16 hours. The reaction mixture was poured into water and the pH was adjusted with HCl (approximately 474.4 μL of 1 M, 0.4744 mmol), then extracted with EtOAc (3×). Organics were combined, washed with water, brine, dried (Na₂SO₄) and evaporated to dryness. The material was taken up in 1:1 MeOH DMSO and purification by HPLC (1-99% ACN in water (HCl modifier)) gave N-[4-(2,6-dimethylphenyl)-6-(4-piperazin-1-ylphenoxy)pyrimidin-2-yl]-1-methyl-pyrazole-4-sulfonamide (hydrochloride salt) (44.2 mg, 67%). ¹H NMR (400 MHz, Deuterium Oxide) δ 7.32-7.24 (m, 2H), 7.23-7.11 (m, 6H), 6.85 (d, J=0.7 Hz, 1H), 6.45 (s, 1H), 3.67 (s, 3H), 3.42 (t, J=5.4 Hz, 4H), 3.38-3.31 (m, 4H), 2.04 (s, 6H). ESI-MS m/z calc. 519.20526, found 520.3 (M+1)⁺; Retention time: 1.14 minutes; LC method A.

### Example 163: Preparation of Compound 452

### Step 1: tert-Butyl 4-(2-chloro-4-hydroxy-phenyl)piperazine-1-carboxylate

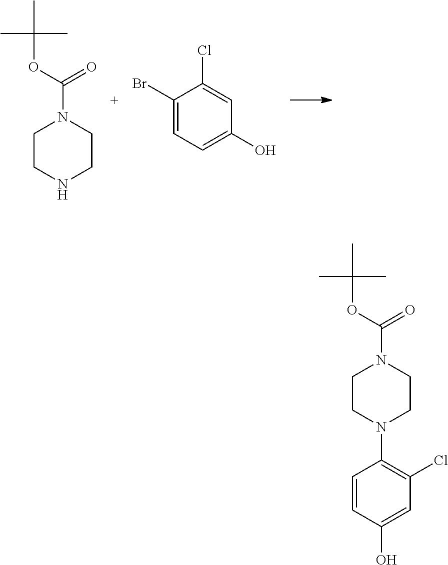 [SMILES: CC(C)(C)OC(=O)N1CCNCC1 ; Oc1ccc(Br)c(Cl)c1 ; CC(C)(C)OC(=O)N1CCN(c2ccc(O)cc2Cl)CC1]

A dried flask was charged with Pd₂(dba)₃ (37.5 mg, 0.04095 mmol), tert-butyl piperazine-1-carboxylate (approximately 362.8 mg, 1.948 mmol) and 4-bromo-3-chloro-phenol (336.6 mg, 1.623 mmol) at room temperature. The flask was capped with a rubber septum, evacuated, and then flushed with nitrogen (three times). Ligand 4,6,11-triisobutyl-1,4,6,11-tetraza-5-phosphabicyclo[3.3.3]undecane (approximately 22.24 mg, 23.07 μL, 0.06492 mmol), toluene (8.115 mL), and LiHMDS (approximately 3.733 mL of 1 M, 3.733 mmol) were added successively and the reaction mixture was heated at 80° C. for 16 hours and then cooled to room temperature. The reaction mixture was poured into aqueous saturated ammonium chloride (15 mL) and ethyl acetate (10 mL) was added. The two layers were separated and the organic layer was washed with brine (5 mL), dried over anhydrous sodium sulfate, filtered, concentrated, and purified by flash column chromatography (12 g silica gel, 0-30% ethyl acetate/hexanes) to give tert-butyl 4-(2-chloro-4-hydroxy-phenyl)piperazine-1-carboxylate. ESI-MS m/z calc. 312.12408, found 313.34 (M+1)⁺; Retention time: 0.61 minutes. LC method D

### Step 2: N-[4-(3-chloro-4-piperazin-1-yl-phenoxy)-6-(2,6-dimethylphenyl)pyrimidin-2-yl]-1-methyl-pyrazole-4-sulfonamide

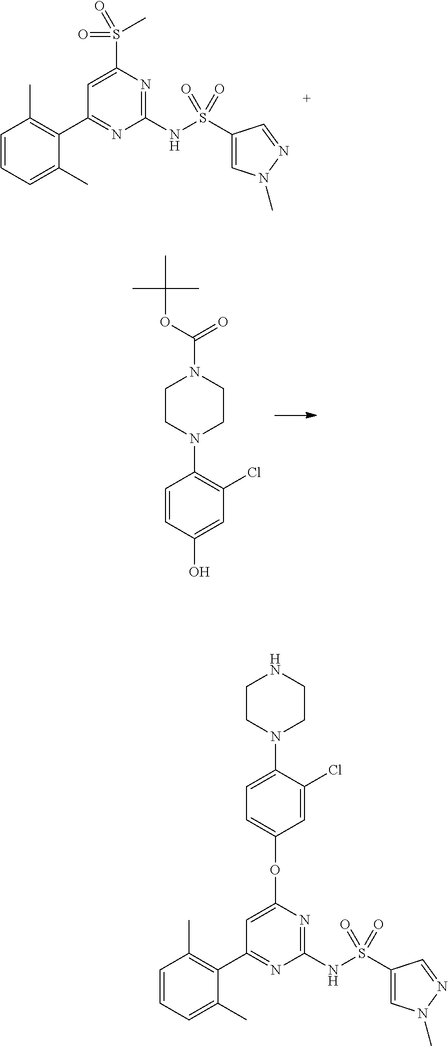 [SMILES: Cc1cccc(C)c1-c1cc(S(C)(=O)=O)nc(NS(=O)(=O)c2cnn(C)c2)n1 ; CC(C)(C)OC(=O)N1CCN(c2ccc(O)cc2Cl)CC1 ; Cc1cccc(C)c1-c1cc(Oc2ccc(N3CCNCC3)c(Cl)c2)nc(NS(=O)(=O)c2cnn(C)c2)n1]

An NMP (600 μL) solution of N-[4-(2,6-dimethylphenyl)-6-methylsulfonyl-pyrimidin-2-yl]-1-methyl-pyrazole-4-sulfonamide (13.0 mg, 0.03084 mmol), cesium carbonate (10.2 mg, 0.03131 mmol), and tert-butyl 4-(2-chloro-4-hydroxy-phenyl)piperazine-1-carboxylate (14.3 mg, 0.04572 mmol) was heated to 110° C. for 1 hour and then diluted with ethyl acetate (1 mL) and water (0.5 mL). The pH of aqueous layer was adjusted to 4 by the addition of concentrated HCl dropwise via syringe. The organic layer was dried over sodium sulfate, filtered, and concentrated to a brown residue. The residue was taken up in TFA (400 μL, 5.192 mmol) and stirred for 16 hours at 40° C. The solution was filtered, concentrated and the resulting residue dissolved in 1.5 mL DMSO, and purified by reverse phase chromatography using a 15 minutes gradient of 20% MeCN in water to 80% MeCN to give N-[4-(3-chloro-4-piperazin-1-yl-phenoxy)-6-(2,6-dimethylphenyl)pyrimidin-2-yl]-1-methyl-pyrazole-4-sulfonamide (hydrochloride salt) (7.3 mg, 40%). ¹H NMR (400 MHz, DMSO-d₆) δ 8.90 (s, 2H), 7.57 (d, J=2.6 Hz, 1H), 7.40-7.29 (m, 2H), 7.28-7.20 (m, 1H), 7.14 (d, J=7.6 Hz, 3H), 6.65 (s, 1H), 3.76 (s, 3H), 3.35-3.25 (m, 4H), 3.25-3.17 (m, 4H), 2.04 (s, 6H). (1H likely overlapped under the water signal). ESI-MS m/z calc. 553.16626, found 554.46 (M+1)⁺; Retention time: 1.19 minutes. LC method A

### Example 164: Preparation of Compound 453

### Step 1: tert-Butyl 4-(4-hydroxy-2-methyl-phenyl)piperazine-1-carboxylate

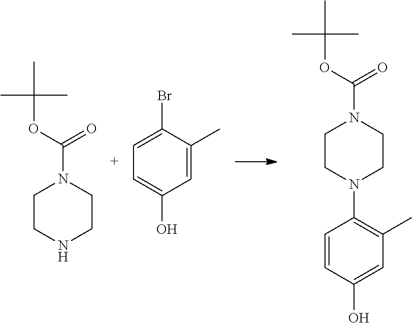

A dried flask was charged with Pd₂(dba)₃ (approximately 25.64 mg, 0.02800 mmol), tert-butyl piperazine-1-carboxylate (300.2 mg, 1.612 mmol), and 4-bromo-3-methyl-phenol (261.9 mg, 1.400 mmol) at room temperature. The flask was capped with a rubber septum, evacuated, and then flushed with nitrogen (three times). Ligand 4,6,11-triisobutyl-1,4,6,11-tetraza-5-phosphabicyclo[3.3.3]undecane (approximately 19.18 mg, 19.90 μL, 0.05600 mmol), toluene (5.460 mL), and LiHMDS (approximately 3.220 mL of 1 M, 3.220 mmol) were added successively and the reaction mixture was heated to 80° C. for 16 hours and then saturated aqueous ammonium chloride solution (10 mL) and ethyl acetate (10 mL) were added. The layers were separated, and the organic layer was washed with water (5 mL) and then brine (5 mL). The organic layer was dried over anhydrous sodium sulfate, filtered, and concentrated in vacuo and then loaded onto a 24 g silica column and eluted with 0 to 30% Ethyl acetate/hexanes to give tert-butyl 4-(4-hydroxy-2-methyl-phenyl)piperazine-1-carboxylate. ESI-MS m/z calc. 292.17868, found 293.34 (M+1)⁺; Retention time: 0.48 minutes.

### Step 2: N-[4-(2,6-Dimethylphenyl)-6-(3-methyl-4-piperazin-1-yl-phenoxy)pyrimidin-2-yl]-1-methyl-pyrazole-4-sulfonamide

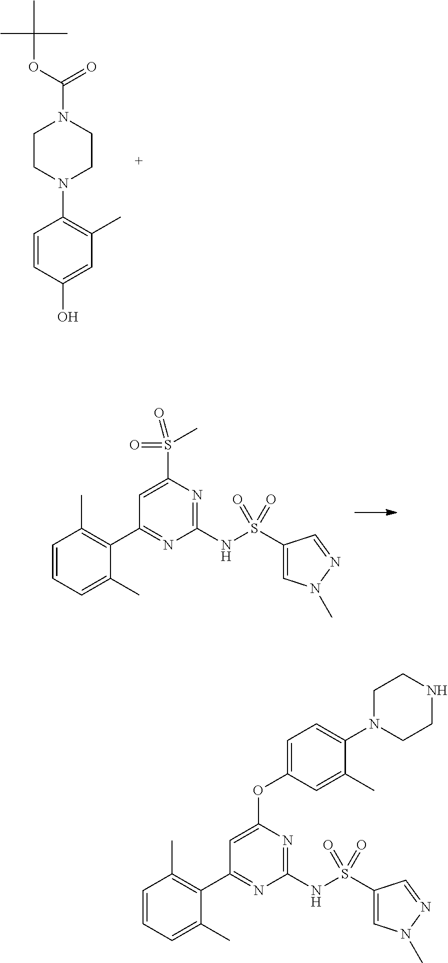 [SMILES: Cc1cc(O)ccc1N1CCN(C(=O)OC(C)(C)C)CC1 ; Cc1cccc(C)c1-c1cc(S(C)(=O)=O)nc(NS(=O)(=O)c2cnn(C)c2)n1 ; Cc1cc(Oc2cc(-c3c(C)cccc3C)nc(NS(=O)(=O)c3cnn(C)c3)n2)ccc1N1CCNCC1]

An NMP (600 μL) solution of tert-butyl 4-(4-hydroxy-2-methyl-phenyl)piperazine-1-carboxylate (37.6 mg, 0.1286 mmol), N-[4-(2,6-dimethylphenyl)-6-methylsulfonyl-pyrimidin-2-yl]-1-methyl-pyrazole-4-sulfonamide (19.3 mg, 0.04579 mmol), and cesium carbonate (16.2 mg, 0.04972 mmol) was heated to 110° C. for 2 hours and then diluted with ethyl acetate (1 mL) and water (0.5 mL). The pH of the aqueous layer was adjusted to 4 by the addition of concentrated HCl via syringe. The organic layer was dried over anhydrous sodium sulfate, filtered, and concentrated to a brown residue. The residue was taken up in TFA (3000 μL, 38.94 mmol) and stirred for 16 hours at 40° C. The solution was filtered, concentrated and the resulting residues dissolved in 1.5 mL DMSO, and purified by reverse phase chromatography using a 15 minutes gradient of 20% MeCN in water to 80% MeCN to give N-[4-(2,6-dimethylphenyl)-6-(3-methyl-4-piperazin-1-yl-phenoxy)pyrimidin-2-yl]-1-methyl-pyrazole-4-sulfonamide (hydrochloride salt) (13.3 mg, 51%). ¹H NMR (400 MHz, DMSO-d₆) δ 9.10 (s, 2H), 7.64 (s, 1H), 7.28-7.17 (m, 3H), 7.17-7.04 (m, 4H), 6.59 (s, 1H), 3.74 (s, 3H), 3.36-3.16 (m, 4H), 3.14-3.00 (m, 4H), 2.32 (s, 3H), 2.04 (s, 6H). ESI-MS m/z calc. 533.2209, found 534.5 (M+1)⁺; Retention time: 1.17 minutes. (LC method A).

### Example 165: Preparation of Compound 454

### Step 1: tert-Butyl 4-(3-chloro-4-hydroxy-phenyl)piperazine-1-carboxylate

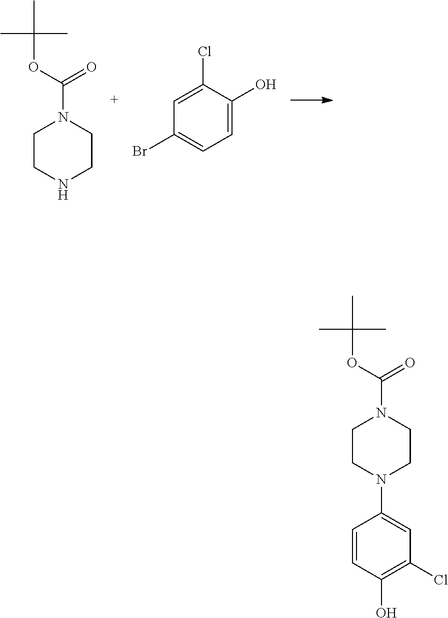 [SMILES: CC(C)(C)OC(=O)N1CCNCC1 ; Oc1ccc(Br)cc1Cl ; CC(C)(C)OC(=O)N1CCN(c2ccc(O)c(Cl)c2)CC1]

A dried flask was charged with Pd₂(dba)₃ (29.3 mg, 0.03200 mmol), tert-butyl piperazine-1-carboxylate (334.8 mg, 1.798 mmol), and 4-bromo-2-chloro-phenol (300 mg, 1.446 mmol) at room temperature. The flask was capped with a rubber septum, evacuated, and then flushed with nitrogen (three times). Ligand 4,6,11-triisobutyl-1,4,6,11-tetraza-5-phosphabicyclo[3.3.3]undecane (25.7 mg, 0.07504 mmol), toluene (8.022 mL), and LiHMDS (approximately 3.326 mL of 1 M, 3.326 mmol) were added successively and the reaction mixture was heated at 80° C. for 3 hours and then cooled to room temperature. The reaction mixture was poured into aqueous saturated ammonium chloride (15 mL) and ethyl acetate (10 mL) was added. The two layers were separated and the organic layer was washed with brine (5 mL), dried over anhydrous sodium sulfate, filtered, concentrated, and purified by flash column chromatography (40 g silica gel, 0-50% ethyl acetate/hexanes) to give tert-butyl 4-(3-chloro-4-hydroxy-phenyl)piperazine-1-carboxylate. ESI-MS m/z calc. 312.12408, found 313.3 (M+1)⁺; Retention time: 0.53 minutes. LC method D.

### Step 2: N-[4-(2-chloro-4-piperazin-1-yl-phenoxy)-6-(2,6-dimethylphenyl)pyrimidin-2-yl]-1-methyl-pyrazole-4-sulfonamide

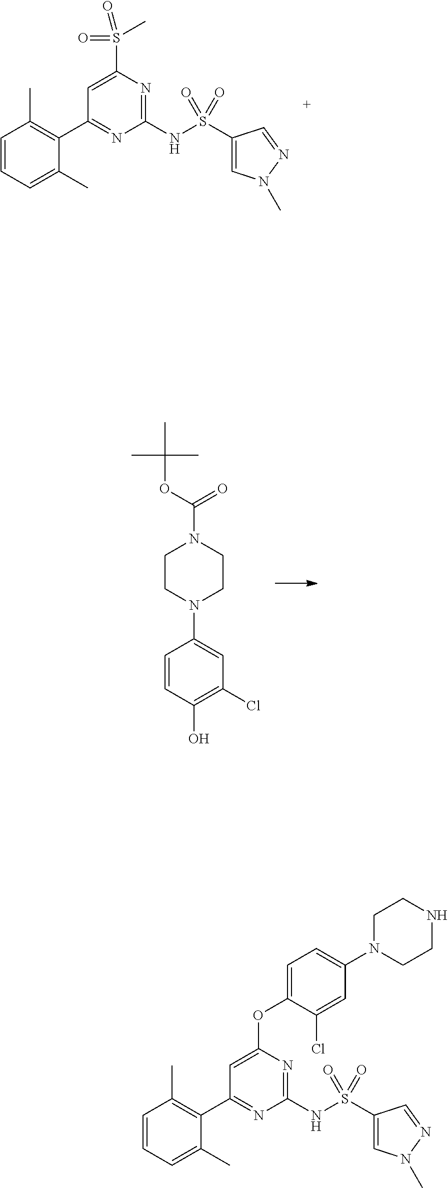 [SMILES: Cc1cccc(C)c1-c1cc(S(C)(=O)=O)nc(NS(=O)(=O)c2cnn(C)c2)n1 ; CC(C)(C)OC(=O)N1CCN(c2ccc(O)c(Cl)c2)CC1 ; Cc1cccc(C)c1-c1cc(Oc2ccc(N3CCNCC3)cc2Cl)nc(NS(=O)(=O)c2cnn(C)c2)n1]

An NMP (600 μL) solution of N-[4-(2,6-dimethylphenyl)-6-methylsulfonyl-pyrimidin-2-yl]-1-methyl-pyrazole-4-sulfonamide (19.5 mg, 0.04626 mmol), tert-butyl 4-(3-chloro-4-hydroxy-phenyl)piperazine-1-carboxylate (41.6 mg, 0.1330 mmol), and cesium carbonate (61.3 mg, 0.1881 mmol)was heated to 110° C. for 2 hours and then diluted with ethyl acetate (1 mL) and water (0.5 mL). The pH of aqueous layer was adjusted to 4 by the addition of concentrated HCl dropwise via syringe. The organic layer was dried over anhydrous sodium sulfate, filtered, and concentrated to a brown residue. The residue was taken up in TFA (3000 μL, 38.94 mmol) and stirred for 16 hours at 40° C. The solution was filtered, concentrated and the resulting residue dissolved in 1.5 mL DMSO, and purified by reverse phase chromatography using a 15 minutes gradient of 20% MeCN in water to 80% MeCN to give N-[4-(2-chloro-4-piperazin-1-yl-phenoxy)-6-(2,6-dimethylphenyl)pyrimidin-2-yl]-1-methyl-pyrazole-4-sulfonamide (hydrochloride salt) (14.3 mg, 52%). ¹H NMR (400 MHz, DMSO-d₆) δ 9.11 (s, 2H), 7.82 (s, 1H), 7.37 (d, J=9.0 Hz, 1H), 7.27-7.23 (m, 3H), 7.15-7.09 (m, 3H), 6.62 (s, 1H), 3.80 (s, 3H), 3.47-3.44 (m, 4H), 3.24-3.17 (m, 4H), 2.02 (s, 6H). ESI-MS m/z calc. 553.16626, found 554.42 (M+1)⁺; Retention time: 1.17 minutes, LC method A.

### Example 166: Preparation of Compound 455

### Step 1: tert-Butyl 4-(4-hydroxy-3-methyl-phenyl)piperazine-1-carboxylate

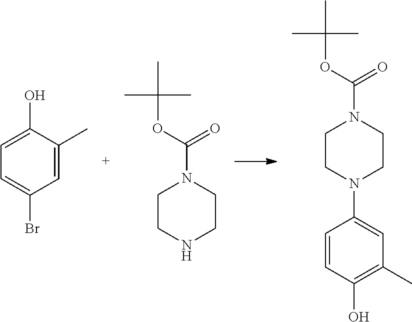

A dried flask was charged with Pd₂(dba)₃ (24.2 mg, 0.02643 mmol), 4-bromo-2 -methyl-phenol (239.7 mg, 1.282 mmol), and tert-butyl piperazine-1-carboxylate (284.8 mg, 1.529 mmol) at room temperature. The flask was capped with a rubber septum, evacuated, and then flushed with nitrogen (three times). Ligand 4,6,11-triisobutyl-1,4,6,11-tetraza-5-phosphabicyclo[3.3.3]undecane (approximately 17.56 mg, 18.22 μL, 0.05128 mmol), toluene (5 mL), and LiHMDS (approximately 2.949 mL of 1 M, 2.949 mmol) were added successively and the reaction mixture was heated to 80° C. for 16 hours and then a saturated aqueous ammonium chloride solution (10 mL) and ethyl acetate (10 mL) were added. The layers were separated, and the organic layer was washed with water (5 mL) and then brine (5 mL). The organic layer was dried over anhydrous sodium sulfate, filtered, and concentrated in vacuo and then loaded onto a 24 g silica column and eluted with 0 to 30% Ethyl acetate/hexanes to give tert-butyl 4-(4-hydroxy-3-methyl-phenyl)piperazine-1-carboxylate. ESI-MS m/z calc. 292.17868, found 293.34 (M+1)⁺; Retention time: 0.38 minutes

### Step 2: N-[4-(2,6-Dimethylphenyl)-6-(2-methyl-4-piperazin-1-yl-phenoxy)pyrimidin-2-yl]-1-methyl-pyrazole-4-sulfonamide

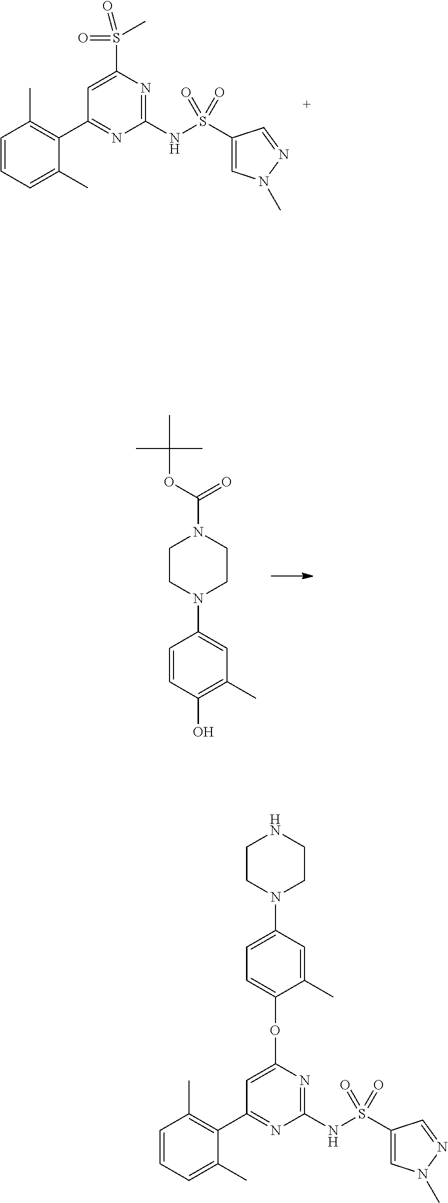 [SMILES: Cc1cccc(C)c1-c1cc(S(C)(=O)=O)nc(NS(=O)(=O)c2cnn(C)c2)n1 ; Cc1cc(N2CCN(C(=O)OC(C)(C)C)CC2)ccc1O ; Cc1cc(N2CCNCC2)ccc1Oc1cc(-c2c(C)cccc2C)nc(NS(=O)(=O)c2cnn(C)c2)n1]

An NMP (0.6 mL) solution of N-[4-(2,6-dimethylphenyl)-6-methylsulfonyl-pyrimidin-2-yl]-1-methyl-pyrazole-4-sulfonamide (22.1 mg, 0.05243 mmol), tert-butyl 4-(4-hydroxy-3-methyl-phenyl)piperazine-1-carboxylate (47.9 mg, 0.1638 mmol), and cesium carbonate (17.1 mg, 0.05 mmol) was heated to 110° C. for 2 hours and then diluted with ethyl acetate (1 mL) and water (0.5 mL). The pH of the aqueous layer was adjusted to 4 by the addition of concentrated HCl dropwise via syringe. The organic layer was dried over anhydrous sodium sulfate, filtered, and concentrated to a brown residue. The residue was taken up in 20% TFA/DCM (v/v, 3 mL) and stirred for 16 hours at 40° C. and then the solution was filtered, concentrated on the rotavapor and the resulting residue was dissolved in 1.5 mL DMSO, and purified by reverse phase chromatography using a 15 minute gradient of 20% MeCN in water to 80% MeCN to give N-[4-(2,6-dimethylphenyl)-6-(2-methyl-4-piperazin-1-yl-phenoxy)pyrimidin-2-yl]-1-methyl-pyrazole-4-sulfonamide (hydrochloride salt) (17.6 mg, 59%).

¹H NMR (400 MHz, DMSO-d₆) δ 9.05 (s, 2H), 7.57 (s, 1H), 7.29-7.20 (m, 1H), 7.18-7.10 (m, 4H), 7.05 (d, J=2.9 Hz, 1H), 6.97 (dd, J=8.8, 3.0 Hz, 1H), 6.54 (s, 1H), 3.77 (s, 3H), 3.43 -3.36 (m, 4H), 3.26-3.19 (m, 4H), 2.11 (s, 3H), 2.04 (s, 6H). ESI-MS m/z calc. 533.2209, found 534.5 (M+1)⁺; Retention time: 1.14 minutes. LC method A.

### Example 167: Preparation of Compound 456

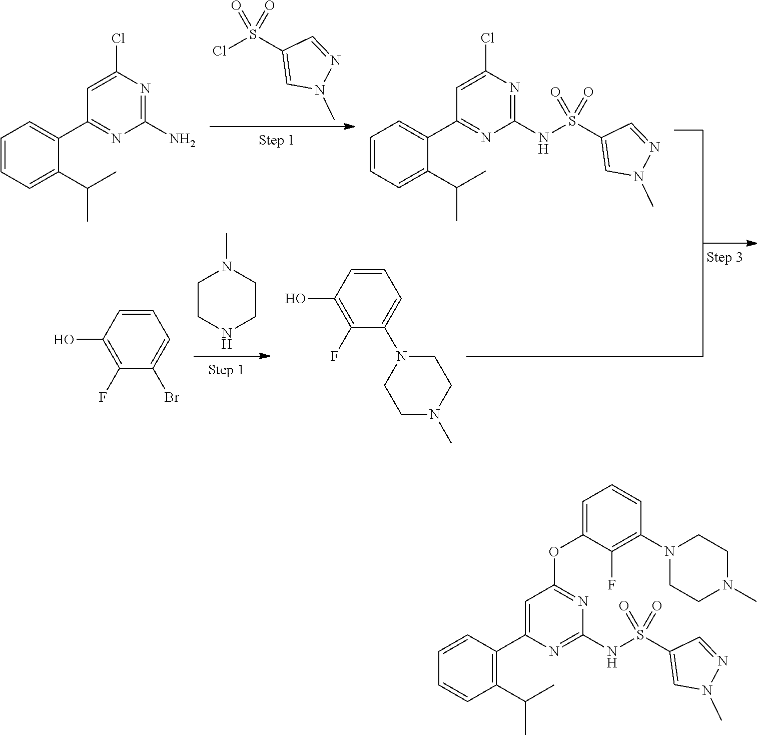

### Step 1: N-[4-chloro-6-(2-isopropylphenyl)pyrimidin-2-yl]-1-methyl-pyrazole-4-sulfonamide

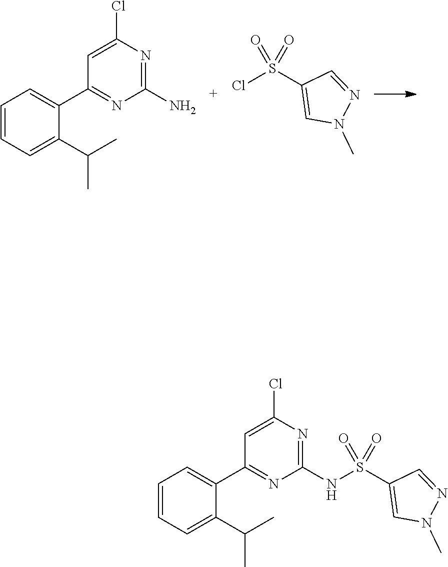

To a suspension of 60% sodium hydride in mineral oil (19.3 g, 0.482 mol) in anhydrous tetrahydrofuran (100 mL) was added solution of 4-chloro-6-(2-isopropylphenyl)pyrimidin-2-amine (42.64 g, 0.172 mol) in anhydrous tetrahydrofuran (200 mL) dropwise at 0° C. The reaction was stirred at room temperature for 1 hour, and then cooled back to 0° C. using an ice bath. A solution of 1-methyl-1H-pyrazole-4-sulfonyl chloride (46.65 g, 0.258 mol) in anhydrous tetrahydrofuran (200 mL) was added to the reaction mixture dropwise at 0° C. The reaction mixture was then stirred at room temperature for 16 hours and poured into 1 N hydrochloric acid (500 mL). Ethyl acetate (200 mL) was added and the two layers were separated. The aqueous layer was extracted with ethyl acetate (500 mL). The combined organic layers were washed with brine (500 mL), dried over anhydrous sodium sulfate and concentrated. The crude product was triturated with ethanol (300 mL) at reflux temperature, and then cooled to 0° C. The product was collected by filtration, washed with ethanol (50 mL), and dried to furnish N-{4-chloro-6-[2-(propan-2-yl)phenyl]pyrimidin-2-yl}-1-methyl-1H-pyrazole-4-sulfonamide (62.76 g, 93%) as a white solid. ESI-MS m/z: calc. 391.1, found 392.0 (M+1)⁺. Retention time: 4.98 minutes. ¹H NMR (250 MHz, CDCl₃) δ (ppm): 8.04 (s, 1H), 8.02 (br, 1H), 7.96 (s, 1H), 7.45 (m, 2H), 7.24 (m, 2H), 7.05 (s, 1H), 3.89 (s, 3H), 3.21 (m, 1H), 1.19 (d, J=6.8 Hz, 6H).

### Step 2: 2-Fluoro-3-(4-methylpiperazin-1-yl)phenol

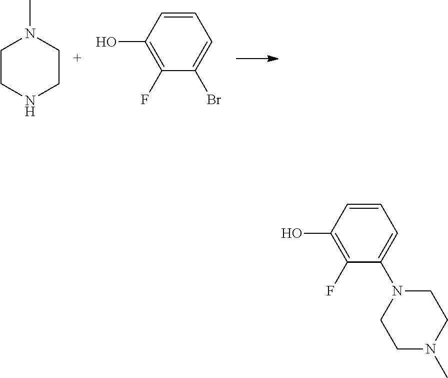

A dioxane (15 mL) mixture of 1-methylpiperazine (950 μL, 8.555 mmol), 3-bromo-2-fluoro-phenol (534.7 mg, 2.800 mmol), sodium tert-butoxide (1.046 g, 10.88 mmol), and chloro(2-di-t-butylphosphino-2′, 4′, 6′-tri-i-propyl-1,1′-biphenyl)[2-(2-aminoethyl)phenyl]palladium(II) [t-BuXPhos Palladacycle Gen. 1] (365.1 mg, 0.5606 mmol) was stirred at room temperature for 2 hours and then HCl (10 mL of 1 M, 10.00 mmol) was added to bring the pH of the aqueous layer to ˜7-8. Ethyl acetate (20 mL) was added and the two layers were separated. The product was extracted from the aqueous layer with ethyl acetate (2×10 mL) and the combined organic layers were dried over anhydrous sodium sulfate, filtered, and concentrated in vacuo. The material was taken up in a minimal amount of dichloromethane and loaded onto a 24 g silica gel column eluted with a gradient of 0-10% MeOH/DCM over 20 minutes to give 2-fluoro-3-(4-methylpiperazin-1-yl)phenol (321.2 mg, 55%). ESI-MS m/z calc. 210.11684, found 211.13 (M+1)⁺; Retention time: 0.49 minutes. LC method A.

### Step 3: N-[4-[2-fluoro-3-(4-methylpiperazin-1-yl)phenoxy]-6-(2-isopropylphenyl)pyrimidin-2-yl]-1-methyl-pyrazole-4-sulfonamide

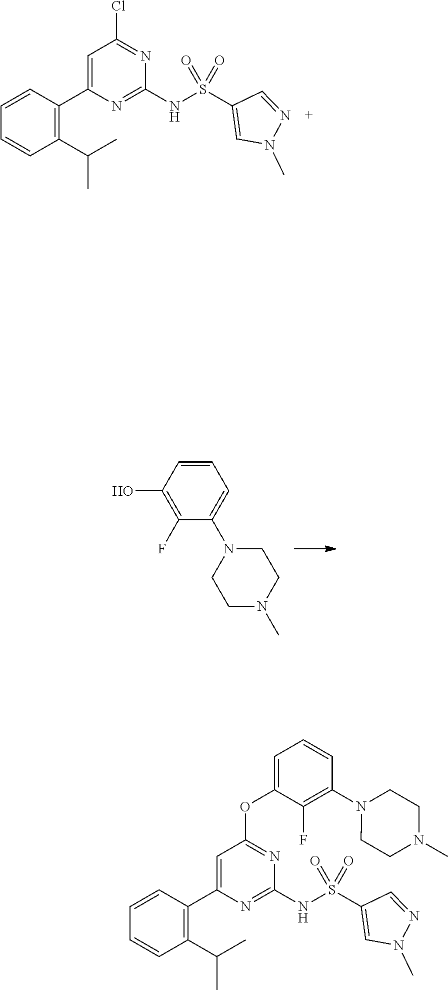 [SMILES: CC(C)c1ccccc1-c1cc(Cl)nc(NS(=O)(=O)c2cnn(C)c2)n1 ; CN1CCN(c2cccc(O)c2F)CC1 ; CC(C)c1ccccc1-c1cc(Oc2cccc(N3CCN(C)CC3)c2F)nc(NS(=O)(=O)c2cnn(C)c2)n1]

A NMP (0.5 mL) mixture of N-[4-chloro-6-(2-isopropylphenyl)pyrimidin-2-yl]-1 -methyl-pyrazole-4-sulfonamide (20.7 mg, 0.05282 mmol), 2-fluoro-3-(4-methylpiperazin-1-yl)phenol (51.2 mg, 0.2435 mmol), and Cs₂CO₃ (80.3 mg, 0.2465 mmol) was stirred at 110° C. for 16 hours and then cooled to room temperature. The solution was filtered and the filtrate dissolved in 0.8 mL MeOH, and purified by reverse phase chromatography using a 15 minutes gradient of 20% MeCN in water to 80% MeCN (HCl modifier) to give N-[4-[2-fluoro-3-(4-methylpiperazin-1-yl)phenoxy]-6-(2-isopropylphenyl)pyrimidin-2-yl]-1-methyl-pyrazole-4-sulfonamide (hydrochloride salt) (6.5 mg, 5%). ESI-MS m/z calc. 565.2271, found 566.2 (M+1)⁺; Retention time: 1.33 minutes. LC method A.

### Example 168: Preparation of Compound 457

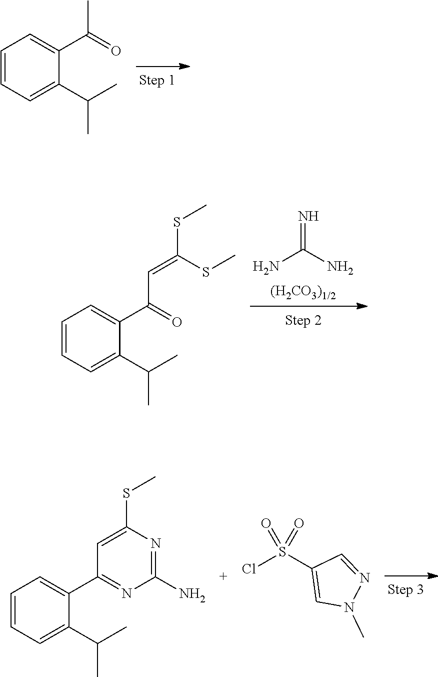

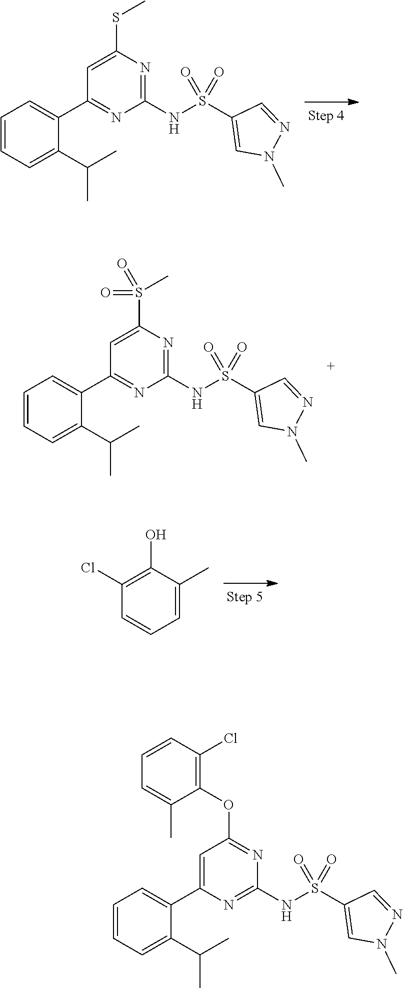

### Step 1: 1-(2-Isopropylphenyl)-3,3-bis(methylsulfanyl)prop-2-en-1-one

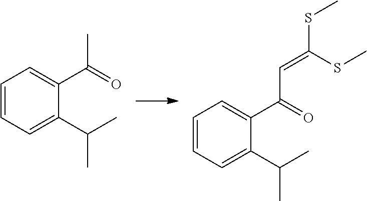 [SMILES: CC(=O)c1ccccc1C(C)C ; CSC(=CC(=O)c1ccccc1C(C)C)SC]

A 500 mL round-bottomed flask equipped with a magnetic stir bar was dried with a heat gun under vacuum and purged with nitrogen; to this was added 1-(2-isopropylphenyl)ethanone (5.13 g, 31.62 mmol). Dry tetrahydrofuran (150 mL) was added and this solution was cooled to 0° C. 60% NaH (2.75 g, 68.76 mmol) was added in two portions, and the reaction mixture was warmed to room temperature over 30 minutes. The mixture was cooled to 0° C., upon which carbon disulfide (2.60 mL, 43.23 mmol) was added. The solution was then warmed to room temperature over 30 minutes. The reaction mixture was cooled to 0° C., upon which iodomethane (5.00 mL, 80.32 mmol) was added, and the mixture was warmed to room temperature over 6 hours. The reaction was quenched by a slow transfer onto ice-cold water (250 mL). The mixture was extracted with ethyl acetate (3×200 mL). The combined organic extracts were washed with water (200 mL) and saturated aqueous sodium chloride solution (200 mL), then dried over sodium sulfate, filtered, and evaporated in vacuo. This oil was purified by silica gel chromatography (80 g of silica, 0 to 15% gradient of ethyl acetate/hexanes) to give a yellow viscous oil, 1-(2-isopropylphenyl)-3,3-bis(methylsulfanyl)prop-2-en-1-one (5.690 g, 68%). ESI-MS m/z calc. 266.0799, found 267.1 (M+1)⁺; Retention time: 0.69 minutes (LC method D).

### Step 2: 4-(2-Isopropylphenyl)-6-methylsulfanyl-pyrimidin-2-amine

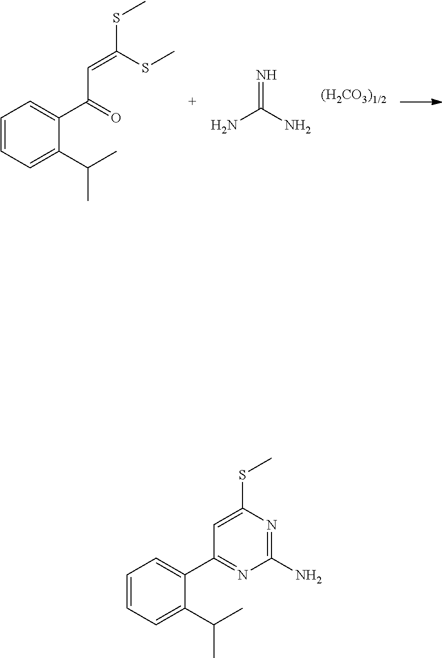

To a heat-gun-dried 250 mL round-bottomed flask equipped with a magnetic stir bar were added 1-(2-isopropylphenyl)-3,3-bis(methylsulfanyl)prop-2-en-1-one (5.690 g, 21.36 mmol), dimethylformamide (100 mL), guanidine carbonate (7.800 g, 43.29 mmol) and potassium carbonate (11.70 g, 84.66 mmol), in this order. This solution was heated at 100° C. for 18 hours. After cooling to room temperature, the flask was opened and water (250 mL) was added. This mixture was extracted with ethyl acetate (3×200 mL), then the organic layers were combined and washed with water (250 mL) and saturated aqueous sodium chloride solution (150 mL), dried over sodium sulfate, filtered, and evaporated in vacuo. This crude product was purified by silica gel chromatography (40 g of silica, 0 to 15% gradient of ethyl acetate/hexanes) to give a yellow solid, 4-(2-isopropylphenyl)-6-methylsulfanyl-pyrimidin-2-amine (3.140 g, 53%). ESI-MS m/z calc. 259.11432, found 260.1 (M+1)⁺; Retention time: 0.46 minutes (LC method D). ¹H NMR (400 MHz, Chloroform-d) δ 7.41-7.35 (m, 2H), 7.27-7.19 (m, 2H), 6.57 (s, 1H), 5.06 (br s, 2H), 3.21 (heptet, J=6.9 Hz, 1H), 2.52 (s, 3H), 1.20 (d, J=6.9 Hz, 6H).

### Step 3: N-[4-(2-isopropylphenyl)-6-methylsulfanyl-pyrimidin-2-yl]-1-methyl-pyrazole-4-sulfonamide

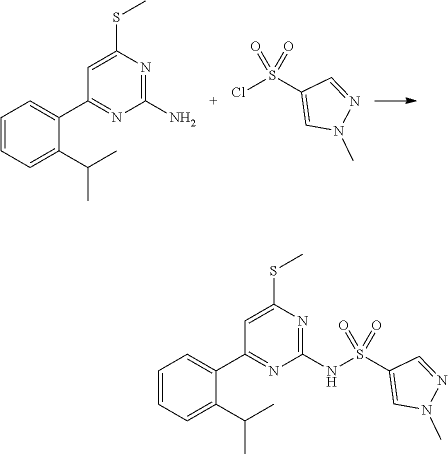

To a heat-gun-dried 20 mL microwave vial equipped with a magnetic stir bar were added 4-(2-isopropylphenyl)-6-methylsulfanyl-pyrimidin-2-amine (654.2 mg, 2.522 mmol) and dimethylformamide (8 mL), and this mixture was cooled to 0° C. 60% NaH (400.0 mg, 10.00 mmol) was added in one portion, and the reaction mixture was warmed to room temperature over 15 minutes. The mixture was cooled to 0° C., upon which 1-methylpyrazole-4-sulfonyl chloride (700.0 mg, 3.876 mmol) was added in one portion. This solution was stirred at room temperature for 4 hours, then quenched by a slow transfer onto ice-cold iN HCl (30 mL). The mixture was extracted with ethyl acetate (3×40 mL). The combined organic extracts were washed with water (50 mL) and saturated aqueous sodium chloride solution (40 mL), then dried over sodium sulfate, filtered, and evaporated in vacuo. This crude product was purified by silica gel chromatography (24 g of silica, 0 to 50% gradient of ethyl acetate/hexanes) to give five batches of product, which contains recovered starting material (24.5%) and the desired product, N-[4-(2-isopropylphenyl)-6-methylsulfanyl-pyrimidin-2-yl]-1-methyl-pyrazole-4-sulfonamide (309.6 mg, 30%). ¹H NMR (400 MHz, Chloroform-d) δ 7.91 (s, 1H), 7.90 (s, 1H), 7.45-7.41 (m, 2H), 7.26-7.22 (m, 1H), 7.22-7.18 (m, 1H), 6.84 (s, 1H), 3.85 (s, 3H), 3.21 (hep, J=6.9 Hz, 1H), 2.55 (s, 3H), 1.20 (d, J=6.9 Hz, 6H). ESI-MS m/z calc. 403.11368, found 404.2 (M+1)⁺; Retention time: 0.62 minutes, LC method D.

### Step 4: N-[4-(2-isopropylphenyl)-6-methylsulfonyl-pyrimidin-2-yl]-1-methyl-pyrazole-4-sulfonamide

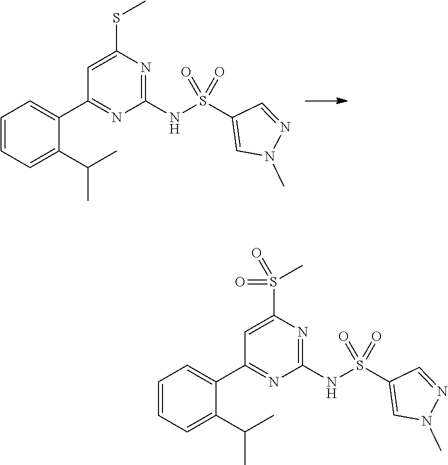 [SMILES: CSc1cc(-c2ccccc2C(C)C)nc(NS(=O)(=O)c2cnn(C)c2)n1 ; CC(C)c1ccccc1-c1cc(S(C)(=O)=O)nc(NS(=O)(=O)c2cnn(C)c2)n1]

To a 20 mL vial equipped with a magnetic stir bar, N-[4-(2-isopropylphenyl)-6-methylsulfanyl-pyrimidin-2-yl]-1-methyl-pyrazole-4-sulfonamide (309.6 mg, 0.7672 mmol) and dichloromethane (7.0 mL) were added, followed by m-CPBA (400.0 mg, 1.785 mmol). This solution was stirred at room temperature for 30 minutes. The reaction mixture was quenched with solid sodium thiosulfate (900.0 mg, 5.692 mmol). This mixture was stirred for another 30 min at room temperature. The reaction mixture was diluted with dichloromethane (7 mL), then washed with water (2×4 mL) and saturated aqueous sodium chloride solution (4 mL). The organic layer was then dried over sodium sulfate, filtered, and evaporated in vacuo. This crude product was purified by silica gel chromatography (12 g of silica, 0 to 70% gradient of ethyl acetate/hexanes) to give N-[4-(2-isopropylphenyl)-6-methylsulfonyl-pyrimidin-2-yl]-1-methyl-pyrazole-4-sulfonamide (309.0 mg, 92%). ESI-MS m/z calc. 435.1035, found 436.2 (M+1)⁺; Retention time: 0.54 minutes, LC method D.

### Step 5: N-[4-(2-chloro-6-methyl-phenoxy)-6-(2-isopropylphenyl)pyrimidin-2-yl]-1 -methyl-pyrazole-4-sulfonamide

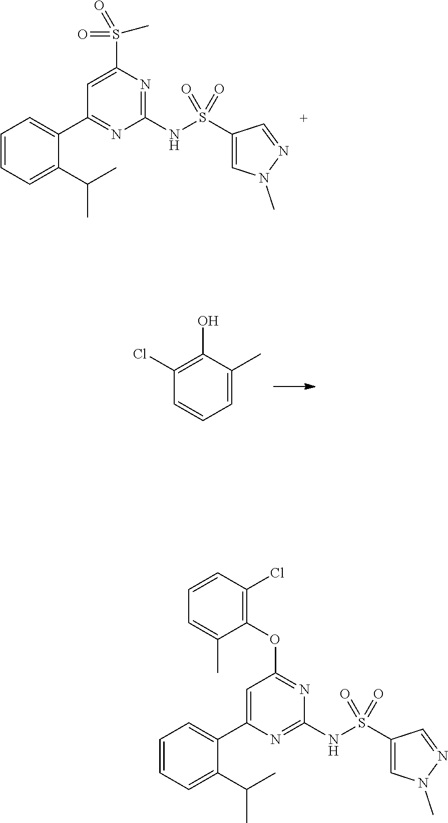

To a 10 mL vial equipped with a magnetic stir bar, N-[4-(2-isopropylphenyl)-6-methylsulfonyl-pyrimidin-2-yl]-1-methyl-pyrazole-4-sulfonamide (15.61 mg, 0.03585 mmol) (15.6 mg, 0.03585 mmol), N-methylpyrrolidinone (500 μL) and 2-chloro-6-methyl-phenol (20.45 mg, 17.03 μL, 0.1434 mmol) were added, followed by potassium carbonate (25.00 mg, 0.1809 mmol) (25.0 mg, 0.1809 mmol). This mixture was stirred at 120° C. for 7 hours. The reaction mixture was then cooled to room temperature, quenched with 1 N HCl (1 mL), and extracted with ethyl acetate (3×1 mL). The combined organic extracts were washed with water (2×2 mL) and saturated aqueous sodium chloride solution (2 mL), then dried over sodium sulfate, filtered, and evaporated in vacuo. Purification by reverse phase HPLC (1-99% acetonitrile in water using HCl as a modifier) gave the desired product N-[4-(2-chloro-6-methyl-phenoxy)-6-(2-isopropylphenyl)pyrimidin-2-yl]-1-methyl-pyrazole-4-sulfonamide (11.7 mg, 66%). ESI-MS m/z calc. 497.12885, found 498.2 (M+1)⁺; Retention time: 1.98 minutes; LC method A.

### Example 169: Preparation of Compound 458

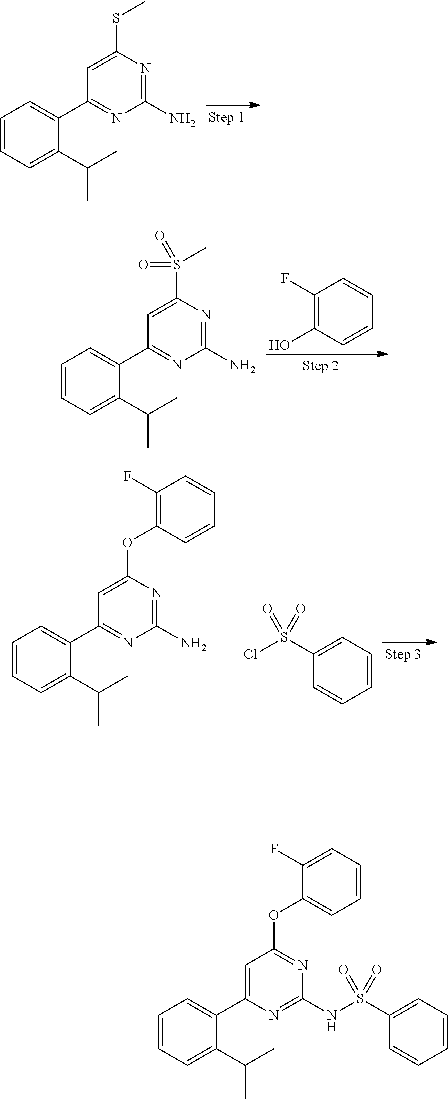

### Step 1: 4-(2-Isopropylphenyl)-6-methylsulfonyl-pyrimidin-2-amine

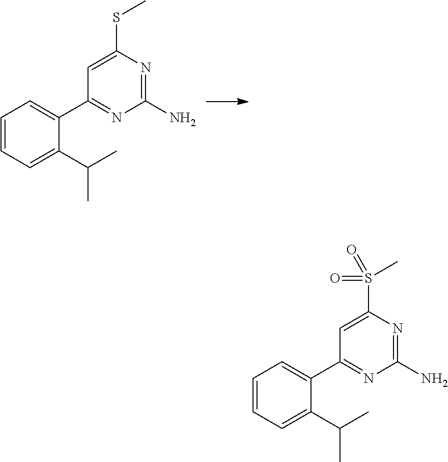 [SMILES: CSc1cc(-c2ccccc2C(C)C)nc(N)n1 ; CC(C)c1ccccc1-c1cc(S(C)(=O)=O)nc(N)n1]

To a 100 mL round-bottomed flask equipped with a magnetic stir bar, 4-(2-isopropylphenyl)-6-methylsulfanyl-pyrimidin-2-amine (2.491 g, 8.932 mmol) and dichloromethane (50 mL) were added, followed by 77% m-CPBA (5.756 g, 25.68 mmol). This mixture was stirred at room temperature for 45 minutes, upon which it was quenched with solid sodium thiosulfate (7.886 g, 49.88 mmol). This mixture was stirred for another 45 min at room temperature. The reaction mixture was diluted with dichloromethane (100 mL), then washed with water (2×100 mL), dried over sodium sulfate, filtered, and evaporated in vacuo. Purification by silica gel chromatography (120 g of silica, 0 to 60% gradient of ethyl acetate/hexanes) gave a yellow foam, 4-(2-isopropylphenyl)-6-methylsulfonyl-pyrimidin-2-amine (2.221 g, 85%). ESI-MS m/z calc. 291.10416, found 292.2 (M+1)⁺; Retention time: 1.35 minutes. LC method A.

### Step 2: 4-(2-fluorophenoxy)-6-(2-isopropylphenyl)pyrimidin-2-amine

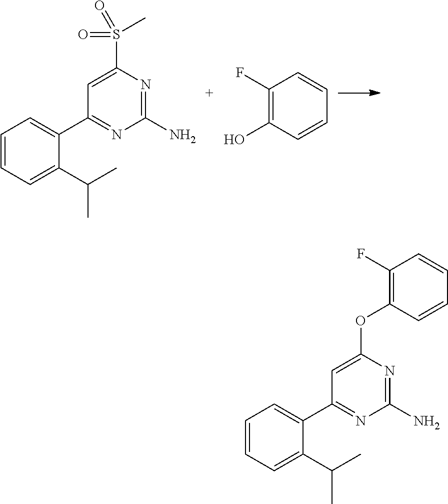 [SMILES: CC(C)c1ccccc1-c1cc(S(C)(=O)=O)nc(N)n1 ; Oc1ccccc1F ; CC(C)c1ccccc1-c1cc(Oc2ccccc2F)nc(N)n1]

To a 100-ml round-bottomed flask, 2-fluorophenol (1.5526 g, 13.850 mmol), K₂CO₃ (2.5654 g, 18.562 mmol), 4-(2-isopropylphenyl)-6-methylsulfonyl-pyrimidin-2-amine (1.6053 g, 5.5095 mmol) and NMP (20 mL) were added. This mixture was stirred at 120° C. for 2 hours. The reaction mixture was then cooled to room temperature, quenched with 1 N HCl (50 mL), and extracted with ethyl acetate (3×50 mL). The combined organic extracts was washed with water (2×80 mL) and saturated aqueous sodium chloride solution (80 mL), then dried over sodium sulfate, filtered, and evaporated in vacuo. A first crop of product was obtained by trituration with dichloromethane and vacuum filtration (0.1854 g). The remaining solution was purified by silica gel chromatography (80 g of silica, 0 to 40% gradient of ethyl acetate/hexanes) to give a white solid (1.0670 g). Obtained product: 4-(2-fluorophenoxy)-6-(2-isopropylphenyl)pyrimidin-2-amine (1.2524 g, 70%)¹H NMR (400 MHz, dimethylsulfoxide-d₆) δ 7.46-7.36 (m, 4H), 7.35-7.22 (m, 4H), 6.77 (s, 2H), 6.23 (s, 1H), 3.23 (hept, J=6.8 Hz, 1H), 1.16 (d, J=6.9 Hz, 6H). ESI-MS m/z calc. 323.1434, found 324.2 (M+1)⁺; Retention time: 1.48 minutes. LC method A

### Step 3: N-[4-(2-Fluorophenoxy)-6-(2-isopropylphenyl)pyrimidin-2-yl]benzenesulfonamide

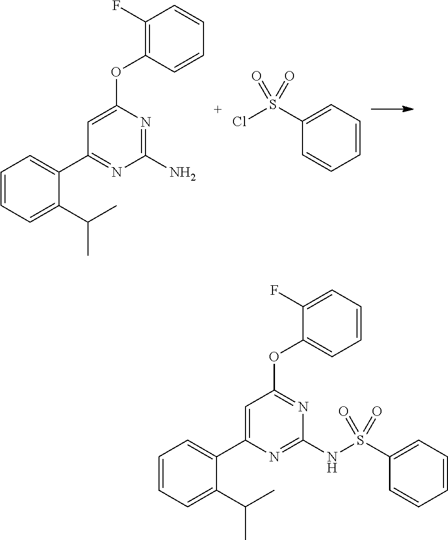 [SMILES: CC(C)c1ccccc1-c1cc(Oc2ccccc2F)nc(N)n1 ; O=S(=O)(Cl)c1ccccc1 ; CC(C)c1ccccc1-c1cc(Oc2ccccc2F)nc(NS(=O)(=O)c2ccccc2)n1]

In a 3-mL vial, 4-(2-fluorophenoxy)-6-(2-isopropylphenyl)pyrimidin-2-amine (25.00 mg, 0.0773 mmol) (25.0 mg, 0.0773 mmol) and benzenesulfonyl chloride (26.49 mg, 19.14 μL, 0.15 mmol) were dissolved in pyridine (800 μL). This mixture was heated at 90° C. for 24 hours, after which it was cooled to room temperature and diluted with ethyl acetate (1 mL). This solution was washed with 1 N HCl (3×1 mL), filtered, and purified by reverse phase HPLC (1-99% acetonitrile in water using HCl as a modifier) to give N-[4-(2-fluorophenoxy)-6-(2-isopropylphenyl)pyrimidin-2-yl]benzenesulfonamide (15.1 mg, 42%). ESI-MS m/z calc. 463.1366, found 464.2 (M+1)⁺; Retention time: 2.07 minutes; LC method A. ¹H NMR (400 MHz, dimethylsulfoxide-d₆) δ 11.85 (s, 1H, D20 exchangeable), 7.63-7.33 (m, 11H), 7.31-7.18 (m, 2H), 6.82 (s, 1H), 3.23-2.99 (m, 1H), 1.13 (d, J=6.8 Hz, 6H) Example 170: Preparation of Compound 459

### Step 1: N-[4-(2-isopropylphenyl)-6-methylsulfanyl-pyrimidin-2-yl]-1-methyl-pyrazole-4-sulfonamide

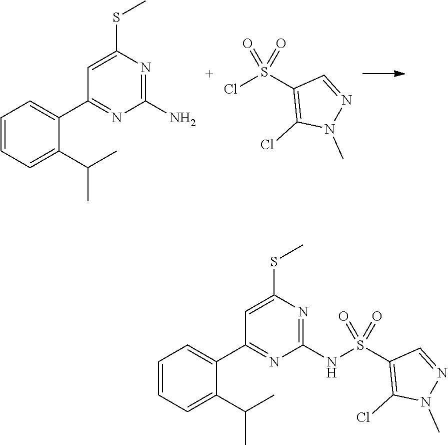 [SMILES: CSc1cc(-c2ccccc2C(C)C)nc(N)n1 ; Cn1ncc(S(=O)(=O)Cl)c1Cl ; CSc1cc(-c2ccccc2C(C)C)nc(NS(=O)(=O)c2cnn(C)c2Cl)n1]

To a heat-gun-dried 20 mL microwave vial equipped with a magnetic stir bar were added 4-(2-isopropylphenyl)-6-methylsulfanyl-pyrimidin-2-amine (654.2 mg, 2.522 mmol) and dimethylformamide (8 mL), and this mixture was cooled to 0° C. 60% NaH (400.0 mg, 10.00 mmol) was added in one portion, and the reaction mixture was warmed to room temperature over 15 minutes. The mixture was cooled to 0° C., upon which 1-methylpyrazole-4-sulfonyl chloride (700.0 mg, 3.876 mmol) was added in one portion. This solution was stirred at room temperature for 4 h, then quenched by a slow transfer onto ice-cold iN HCl (30 mL). The mixture was extracted with ethyl acetate (3×40 mL). The combined organic extracts was washed with water (50 mL) and saturated aqueous sodium chloride solution (40 mL), then dried over sodium sulfate, filtered, and evaporated in vacuo. This crude product was purified by silica gel chromatography (24 g of silica, 0 to 50% gradient of ethyl acetate/hexanes) to give five batches of product, which contains recovered starting material (24.5%), 5-chloro-N-[4-(2-isopropylphenyl)-6-methylsulfanyl-pyrimidin-2-yl]-1-methyl-pyrazole-4-sulfonamide (139.8 mg, 13%). ¹H NMR (400 MHz, Chloroform-d) δ 7.99 (s, 1H), 7.44-7.40 (m, 2H), 7.26-7.21 (m, 1H), 7.21-7.17 (m, 1H), 6.84 (s, 1H), 3.86 (s, 3H), 3.17 (hept, J=6.9 Hz, 1H), 2.53 (s, 3H), 1.19 (d, J=6.9 Hz, 6H). ESI-MS m/z calc. 437.0747, found 438.1 (M+1)⁺; Retention time: 0.65 minutes. LC method D.

### Step 2: 5-Chloro-N-[4-(2-isopropylphenyl)-6-methylsulfonyl-pyrimidin-2-yl]-1 -methyl-pyrazole-4-sulfonamide

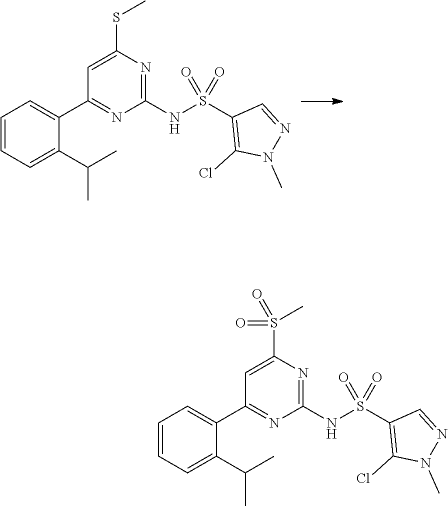 [SMILES: CSc1cc(-c2ccccc2C(C)C)nc(NS(=O)(=O)c2cnn(C)c2Cl)n1 ; CC(C)c1ccccc1-c1cc(S(C)(=O)=O)nc(NS(=O)(=O)c2cnn(C)c2Cl)n1]

To a 20 mL vial equipped with a magnetic stir bar, 5-chloro-N-[4-(2-isopropylphenyl)-6-methylsulfanyl-pyrimidin-2-yl]-1-methyl-pyrazole-4-sulfonamide (124.8 mg, 0.2850 mmol) and dichloromethane (3.0 mL) were added, followed by m-chloroperbenzoic acid (150.0 mg, 0.6693 mmol). This solution was stirred at room temperature for 1 hour. The reaction mixture was quenched with solid sodium thiosulfate (300.0 mg, 1.897 mmol). This mixture was stirred for another 1 hour at room temperature. The reaction mixture was diluted with dichloromethane (7 mL), then washed with water (2×4 mL) and saturated aqueous sodium chloride solution (4 mL). The organic layer was then dried over sodium sulfate, filtered, and evaporated in vacuo. This crude product was purified by silica gel chromatography (4 g of silica, 0 to 70% gradient of ethyl acetate/hexanes) to give 5-chloro-N-[4-(2-isopropylphenyl)-6-methylsulfonyl-pyrimidin-2-yl]-1-methyl-pyrazole-4-sulfonamide (109.4 mg, 82%). ESI-MS m/z calc. 469.0645, found 470.1 (M+1)⁺; Retention time: 0.59 minutes. LC method D.

### Step 3: 5-Chloro-N-[4-(2-chloro-6-methyl-phenoxy)-6-(2-isopropylphenyl)pyrimidin-2-yl]-1-methyl-pyrazole-4-sulfonamide

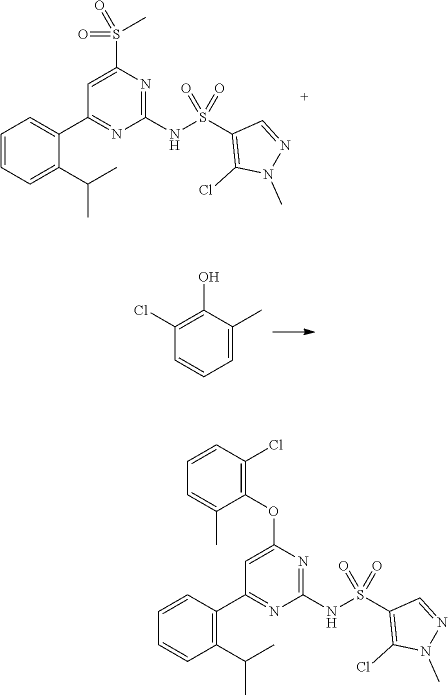

To a 10 mL vial equipped with a magnetic stir bar, N-methylpyrrolidinone (200 μL) and 2-chloro-6-methyl-phenol (21.39 mg, 0.15 mmol) were added, followed by 60% NaH (5.999 mg, 0.15 mmol) (6.0 mg, 0.15 mmol). This slurry was stirred for 5 minutes at room temperature, after which a solution of 5-chloro-N-[4-(2-isopropylphenyl)-6-methylsulfonyl-pyrimidin-2-yl]-1-methyl-pyrazole-4-sulfonamide (20.00 mg, 0.042557 mmol) (20.0 mg, 0.0426 mmol) in N-methylpyrrolidinone (300 μL) was added. After 20 min at room temperature, the reaction was quenched with 1 N HCl (1 mL), and ethyl acetate (3 mL) was added. After the layers had separated, the aqueous phase was discarded, and the organic layer was washed with water (2×2 mL) and saturated aqueous sodium chloride solution (2 mL), then dried over sodium sulfate, filtered, and evaporated in vacuo. Purification by reverse phase HPLC (1-99% acetonitrile in water using HCl as a modifier) gave the desired product 5-chloro-N-[4-(2-chloro-6-methyl-phenoxy)-6-(2-isopropylphenyl)pyrimidin-2-yl]-1-methyl-pyrazole-4-sulfonamide (11.9 mg, 53%). ESI-MS m/z calc. 531.08984, found 532.1 (M+1)⁺; Retention time: 2.03 minutes; LC method A.

### Example 171: Characterization of Compounds 460-472

The compounds in the following tables were prepared in a manner analogous to that described above using commercially available reagents and intermediates described herein.

| Compound | | LCMS Rt | Calc. | | LCMS |
|---|---|---|---|---|---|
| number | Structure | (min) | mass | M + 1 | Method |
| 460 | 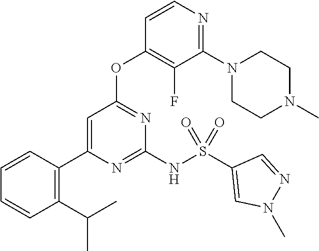 [SMILES: CC(C)c1ccccc1-c1cc(Oc2ccnc(N3CCN(C)CC3)c2F)nc(NS(=O)(=O)c2cnn(C)c2)n1] | 1.3 | 566.222 | 567.1 | A |
| 461 | 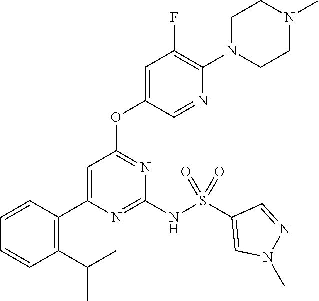 [SMILES: CC(C)c1ccccc1-c1cc(Oc2cnc(N3CCN(C)CC3)c(F)c2)nc(NS(=O)(=O)c2cnn(C)c2)n1] | 1.28 | 566.222 | 567.2 | A |
| 462 | 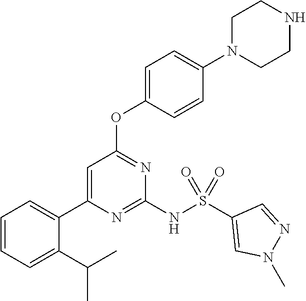 [SMILES: CC(C)c1ccccc1-c1cc(Oc2ccc(N3CCNCC3)cc2)nc(NS(=O)(=O)c2cnn(C)c2)n1] | 1.35 | 533.221 | 534.5 | A |
| 463 | 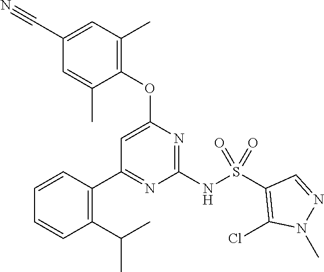 [SMILES: Cc1cc(C#N)cc(C)c1Oc1cc(-c2ccccc2C(C)C)nc(NS(=O)(=O)c2cnn(C)c2Cl)n1] | 1.95 | 536.14 | 537.2 | A |
| 464 | 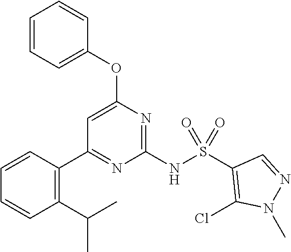 [SMILES: CC(C)c1ccccc1-c1cc(Oc2ccccc2)nc(NS(=O)(=O)c2cnn(C)c2Cl)n1] | 1.87 | 483.113 | 484.2 | A |
| 465 | 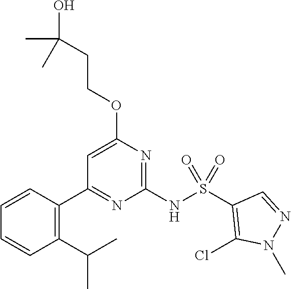 [SMILES: CC(C)c1ccccc1-c1cc(OCCC(C)(C)O)nc(NS(=O)(=O)c2cnn(C)c2Cl)n1] | 1.52 | 493.155 | 494.2 | A |
| 466 | 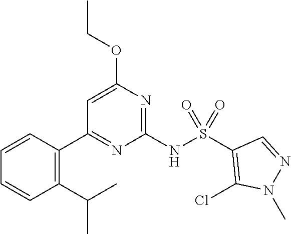 [SMILES: CCOc1cc(-c2ccccc2C(C)C)nc(NS(=O)(=O)c2cnn(C)c2Cl)n1] | 1.62 | 435.113 | 436.1 | A |
| 467 | 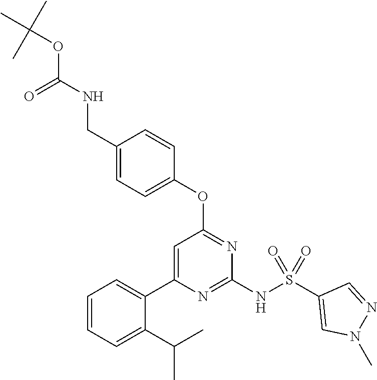 [SMILES: CC(C)c1ccccc1-c1cc(Oc2ccc(CNC(=O)OC(C)(C)C)cc2)nc(NS(=O)(=O)c2cnn(C)c2)n1] | 1.93 | 578.231 | 579.2 | A |
| 468 | 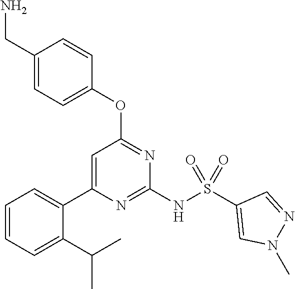 [SMILES: CC(C)c1ccccc1-c1cc(Oc2ccc(CN)cc2)nc(NS(=O)(=O)c2cnn(C)c2)n1] | 1.19 | 478.179 | 479.2 | A |
| 469 | 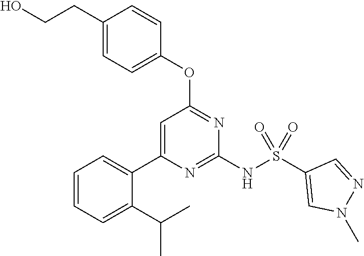 [SMILES: CC(C)c1ccccc1-c1cc(Oc2ccc(CCO)cc2)nc(NS(=O)(=O)c2cnn(C)c2)n1] | 1.59 | 493.178 | 494.2 | A |
| 470 | 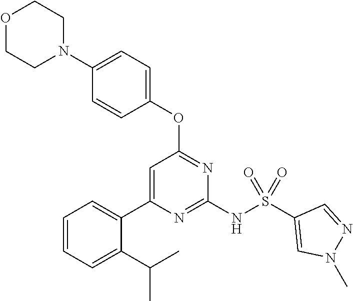 [SMILES: CC(C)c1ccccc1-c1cc(Oc2ccc(N3CCOCC3)cc2)nc(NS(=O)(=O)c2cnn(C)c2)n1] | 1.68 | 534.205 | 535.2 | A |
| 471 | 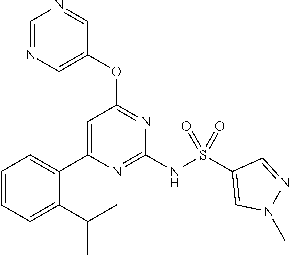 [SMILES: CC(C)c1ccccc1-c1cc(Oc2cncnc2)nc(NS(=O)(=O)c2cnn(C)c2)n1] | 1.45 | 451.143 | 452.2 | A |
| 472 | 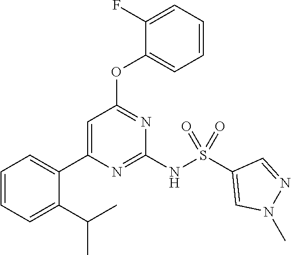 [SMILES: CC(C)c1ccccc1-c1cc(Oc2ccccc2F)nc(NS(=O)(=O)c2cnn(C)c2)n1] | 1.84 | 467.143 | 468.2 | A |

### Example 172: Preparation of Compound 473

### Step 1: N-[4-(2-isopropylphenyl)-6-methylsulfonyl-pyrimidin-2-yl]-1-methyl-pyrazole-4-sulfonamide

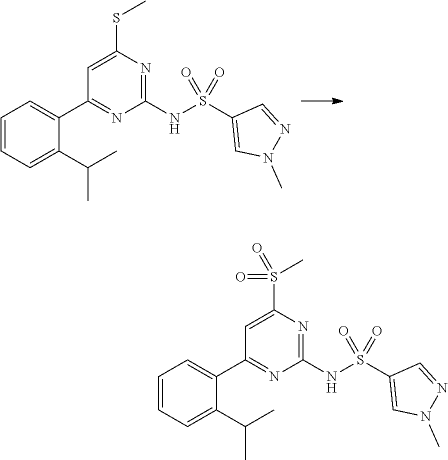 [SMILES: CSc1cc(-c2ccccc2C(C)C)nc(NS(=O)(=O)c2cnn(C)c2)n1 ; CC(C)c1ccccc1-c1cc(S(C)(=O)=O)nc(NS(=O)(=O)c2cnn(C)c2)n1]

To a 20 mL vial equipped with a magnetic stir bar, N-[4-(2-isopropylphenyl)-6-methylsulfanyl-pyrimidin-2-yl]-1-methyl-pyrazole-4-sulfonamide (309.6 mg, 0.7672 mmol) and dichloromethane (7.0 mL) were added, followed by m-CPBA (400.0 mg, 1.785 mmol). This solution was stirred at room temperature for 30 minutes. The reaction mixture was quenched with solid sodium thiosulfate (900.0 mg, 5.692 mmol). This mixture was stirred for another 30 minutes at room temperature. The reaction mixture was diluted with dichloromethane (7 mL), then washed with water (2×4 mL) and saturated aqueous sodium chloride solution (4 mL). The organic layer was then dried over sodium sulfate, filtered, and evaporated in vacuo. This crude product was purified by silica gel chromatography (12 g of silica, 0 to 7000 gradient of ethyl acetate/hexanes) to give N-[4-(2-isopropylphenyl)-6-methylsulfonyl-pyrimidin-2-yl]-1-methyl-pyrazole-4-sulfonamide (309.0 mg, 92% ) ESI-MS m/z calc. 435.1035, found 436.2 (M+1)⁺; Retention time: 0.54 minutes, LC method D.

### Step 2: N-[4-(3-cyanopropoxy)-6-(2-isopropylphenyl)pyrimidin-2-yl]-1-methyl-pyrazole-4-sulfonamide

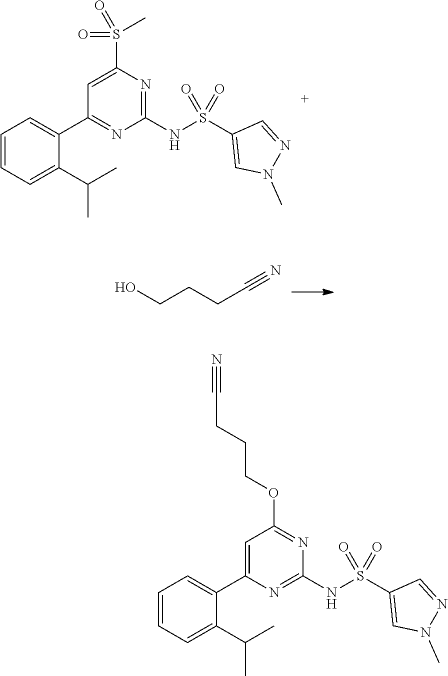 [SMILES: CC(C)c1ccccc1-c1cc(S(C)(=O)=O)nc(NS(=O)(=O)c2cnn(C)c2)n1 ; N#CCCCO ; CC(C)c1ccccc1-c1cc(OCCCC#N)nc(NS(=O)(=O)c2cnn(C)c2)n1]

To a 10 mL vial equipped with a magnetic stir bar, N-methylpyrrolidinone (200 μL) and 4-hydroxybutanenitrile (17.02 mg, 0.2 mmol) were added, followed by 60% NaH (7.999 mg, 0.2 mmol) (8.0 mg, 0.20 mmol). This slurry was stirred for 5 minutes at room temperature, after which a solution of N-[4-(2-isopropylphenyl)-6-methylsulfonyl-pyrimidin-2-yl]-1-methyl-pyrazole-4-sulfonamide (15.00 mg, 0.03444 mmol) (15.0 mg, 0.0344 mmol) in N-methylpyrrolidinone (300 μL) was added. After 20 minutes at room temperature, the reaction was quenched with 1 N HCl (1 mL), and ethyl acetate (3 mL) was added. After the layers had separated, the aqueous phase was discarded, and the organic layer was washed with water (2×2 mL) and saturated aqueous sodium chloride solution (2 mL), then dried over sodium sulfate, filtered, and evaporated in vacuo. Purification by reverse phase HPLC (1-99% acetonitrile in water using HCl as a modifier) gave the desired product N-[4-(3-cyanopropoxy)-6-(2-isopropylphenyl)pyrimidin-2-yl]-1-methyl-pyrazole-4-sulfonamide (3.4 mg, 22%). ESI-MS m z calc. 440.16306, found 441.2 (M+1)⁺; Retention time: 1.44 minutes; LC method A.

### Example 173: Preparation of Compound 474

### Step 1: N-[4-(1-adamantyloxy)-6-(2-isopropylphenyl)pyrimidin-2-yl]-1-methyl-pyrazole-4-sulfonamide

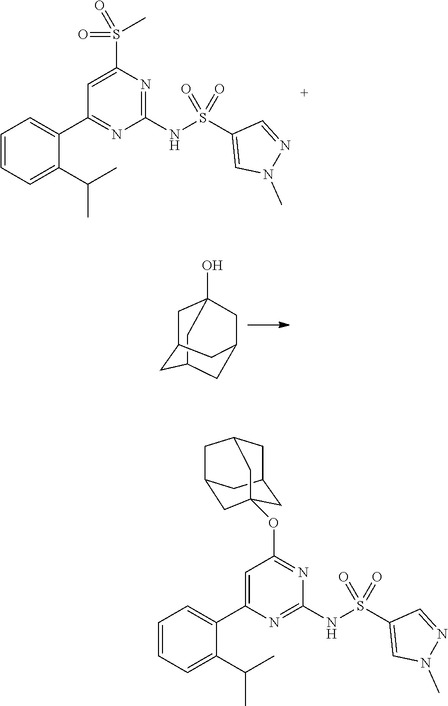 [SMILES: CC(C)c1ccccc1-c1cc(S(C)(=O)=O)nc(NS(=O)(=O)c2cnn(C)c2)n1 ; OC12CC3CC(CC(C3)C1)C2 ; CC(C)c1ccccc1-c1cc(OC23CC4CC(CC(C4)C2)C3)nc(NS(=O)(=O)c2cnn(C)c2)n1]

To a 10 mL vial equipped with a magnetic stir bar, N-methylpyrrolidinone (200 μL) and adamantan-1-ol (30.45 mg, 0.2 mmol) were added, followed by 60% NaH (7.999 mg, 0.2 mmol) (8.0 mg, 0.20 mmol). This slurry was stirred for 5 minutes at room temperature, after which a solution of N-[4-(2-isopropylphenyl)-6-methylsulfonyl-pyrimidin-2-yl]-1-methyl-pyrazole-4-sulfonamide (15.00 mg, 0.03444 mmol) in N-methylpyrrolidinone (300 μL) was added. After 16 hour at room temperature, the reaction was quenched with 1 N HCl (1 mL), and ethyl acetate (3 mL) was added. After the layers had separated, the aqueous phase was discarded, and the organic layer was washed with water (2×2 mL) and saturated aqueous sodium chloride solution (2 mL), then dried over sodium sulfate, filtered, and evaporated in vacuo. Purification by reverse phase HPLC (1-99% acetonitrile in water using HCl as a modifier) gave the desired product N-[4-(1-adamantyloxy)-6-(2-isopropylphenyl)pyrimidin-2-yl]-1-methyl-pyrazole-4-sulfonamide (1.6 mg, 9%). ESI-MS m/z calc. 507.2304, found 508.3 (M+1)⁺; Retention time: 2.16 minutes; LC method A.

### Example 174: Preparation of Compound 475

### Step 1: N-[4-(3-hydroxy-2,2-dimethyl-propoxy)-6-(2-isopropylphenyl)pyrimidin-2-yl]-1-methyl-pyrazole-4-sulfonamide

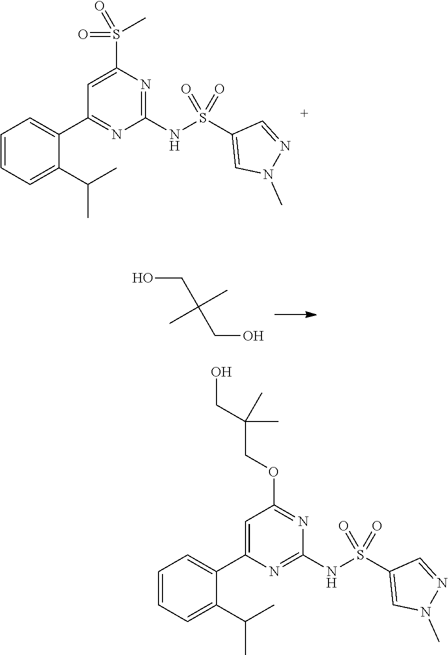

To a 10 mL vial equipped with a magnetic stir bar, N-methylpyrrolidinone (200 μL) and 2,2-dimethylpropane-1,3-diol (20.83 mg, 0.2 mmol) were added, followed by 60% NaH (8.0 mg, 0.20 mmol). This slurry was stirred for 5 minutes at room temperature, after which a solution of N-[4-(2-isopropylphenyl)-6-methylsulfonyl-pyrimidin-2-yl]-1-methyl-pyrazole-4-sulfonamide (15.0 mg, 0.0344 mmol) in N-methylpyrrolidinone (300 μL) was added. After 20 minutes at room temperature, the reaction was quenched with 1 N HCl (1 mL), and ethyl acetate (3 mL) was added. After the layers had separated, the aqueous phase was discarded, and the organic layer was washed with water (2×2 mL) and saturated aqueous sodium chloride solution (2 mL), then dried over sodium sulfate, filtered, and evaporated in vacuo. Purification by reverse phase HPLC (1-99% acetonitrile in water using HCl as a modifier) gave the desired product N-[4-(3-hydroxy-2,2-dimethyl-propoxy)-6-(2-isopropylphenyl)pyrimidin-2-yl]-1-methyl-pyrazole-4-sulfonamide (6.2 mg, 39%). ESI-MS m/z calc. 459.19403, found 460.2 (M+1)⁺; Retention time: 1.51 minutes; LC method A.

### Example 175: Characterization of Compounds 476-486

The compounds in the following tables were prepared in a manner analogous to that described above using commercially available reagents and intermediates described herein.

| | | LCMS | | | |
|---|---|---|---|---|---|
| Compound | | Rt | Calc. | | LCMS |
| number | Structure | (min) | mass | M + 1 | Method |
| 476 | 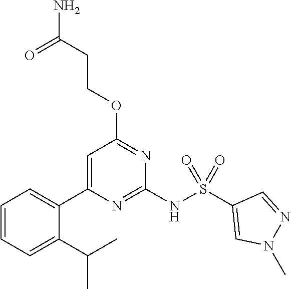 [SMILES: CC(C)c1ccccc1-c1cc(OCCC(N)=O)nc(NS(=O)(=O)c2cnn(C)c2)n1] | 1.13 | 444.158 | 445.2 | A |
| 477 | 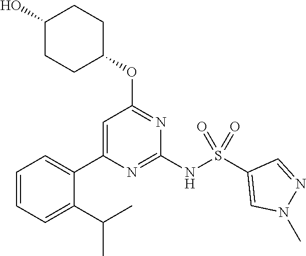 [SMILES: CC(C)c1ccccc1-c1cc(O[C@H]2CC[C@@H](O)CC2)nc(NS(=O)(=O)c2cnn(C)c2)n1] | 1.3 | 471.194 | 472.2 | A |
| 478 | 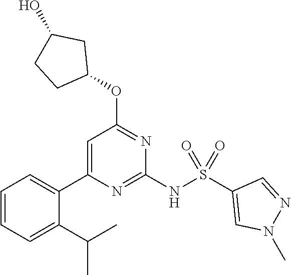 [SMILES: CC(C)c1ccccc1-c1cc(O[C@@H]2CC[C@H](O)C2)nc(NS(=O)(=O)c2cnn(C)c2)n1] | 1.41 | 457.178 | 458.2 | A |
| 479 | 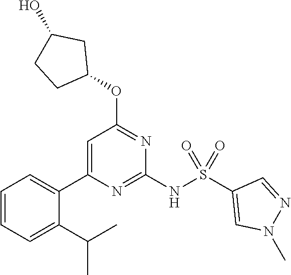 [SMILES: CC(C)c1ccccc1-c1cc(O[C@@H]2CC[C@H](O)C2)nc(NS(=O)(=O)c2cnn(C)c2)n1] | 1.28 | 457.178 | 458.2 | A |
| 480 | 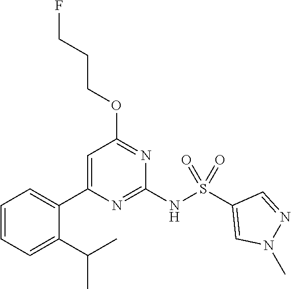 [SMILES: CC(C)c1ccccc1-c1cc(OCCCF)nc(NS(=O)(=O)c2cnn(C)c2)n1] | 1.56 | 433.158 | 434.2 | SD HPLC 1-99 ACN (TFA)-3 min [1] |
| 481 | 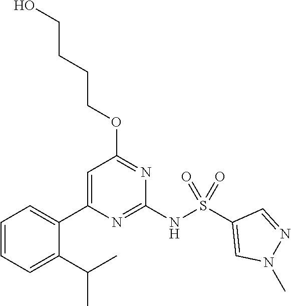 [SMILES: CC(C)c1ccccc1-c1cc(OCCCCO)nc(NS(=O)(=O)c2cnn(C)c2)n1] | 1.34 | 445.178 | 446.2 | A |
| 482 | 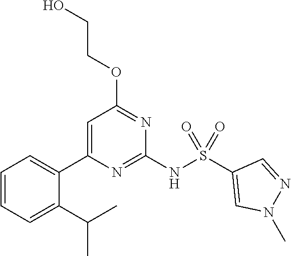 [SMILES: CC(C)c1ccccc1-c1cc(OCCO)nc(NS(=O)(=O)c2cnn(C)c2)n1] | 1.18 | 417.147 | 418.2 | A |
| 483 | 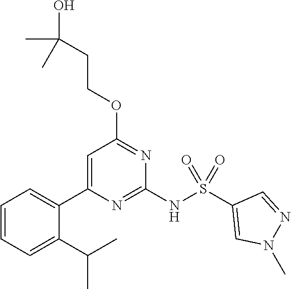 [SMILES: CC(C)c1ccccc1-c1cc(OCCC(C)(C)O)nc(NS(=O)(=O)c2cnn(C)c2)n1] | 1.47 | 459.194 | 460.2 | A |
| 484 | 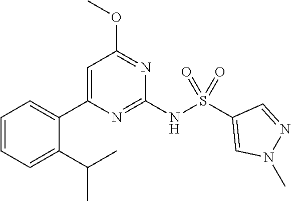 [SMILES: COc1cc(-c2ccccc2C(C)C)nc(NS(=O)(=O)c2cnn(C)c2)n1] | 1.42 | 387.137 | 388.1 | A |
| 485 | 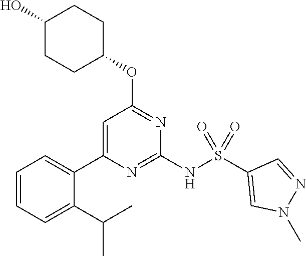 [SMILES: CC(C)c1ccccc1-c1cc(O[C@H]2CC[C@@H](O)CC2)nc(NS(=O)(=O)c2cnn(C)c2)n1] | 1.42 | 471.194 | 472.2 | A |
| 486 | 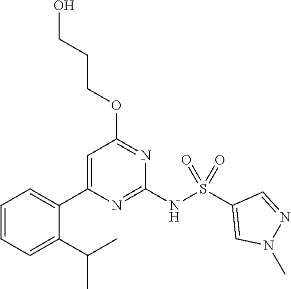 [SMILES: CC(C)c1ccccc1-c1cc(OCCCO)nc(NS(=O)(=O)c2cnn(C)c2)n1] | 1.28 | 431.163 | 432.2 | A |

### Example 176: Preparation of Compound 487

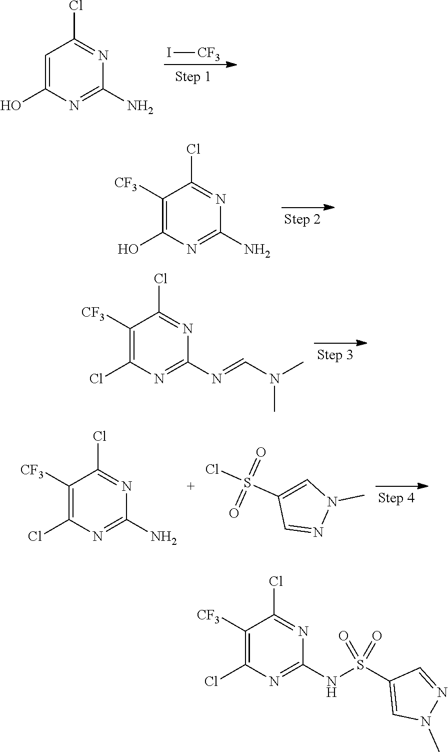

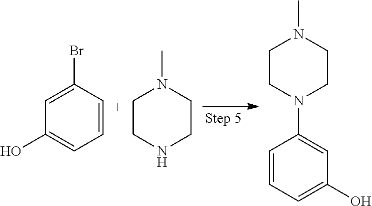

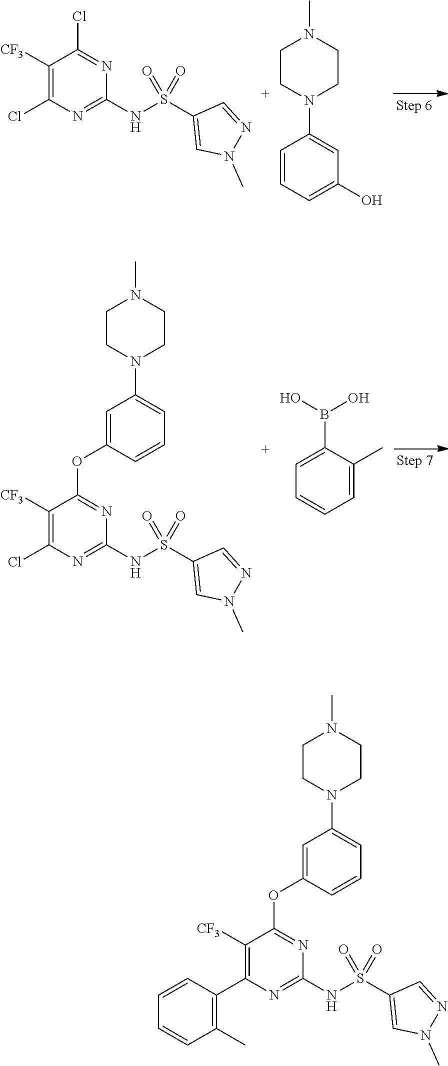

### Step 1: 2-Amino-6-chloro-5-(trifluoromethyl)pyrimidin-4-ol

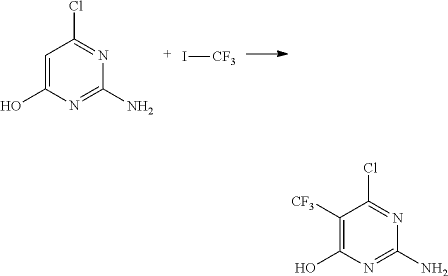 [SMILES: Nc1nc(O)cc(Cl)n1 ; FC(F)(F)I ; Nc1nc(O)c(C(F)(F)F)c(Cl)n1]

An empty 1.0 L flask was cooled in a dry ice bath and trifluoro(iodo)methane (121.15 g, 618.4 mmol) was condensed inside. The flask was then removed from the cold bath and DMSO (200 mL) was added slowly along the inner sides of the flask to give a slurry. This slurry was slowly warmed up until a clear solution was obtained. This solution was then added to a three-neck 1.0 L flask containing a solution of 2-amino-6-chloro-pyrimidin-4-ol (32.20 g, 221.2 mmol) in DMSO (400 mL) at room temperature. Then, a solution of ferrous sulfate was added slowly (66 mL of a roughly 1 M aqueous solution, prepared by dissolving 27.8 g of ferrous sulfate heptahydrate in 100 mL of water, 50 mmol) and the temperature, monitored with a probe inside the reaction, rose a few degrees. To this solution was then added dropwise and carefully, aqueous hydrogen peroxide (38 mL of a 35% aqueous solution, 391 mmol) at such a rate that at the end of the addition, the internal temperature was at 41° C. (over 4 hours). After another 30 minutes, the crude mixture was added equally (about 400 mL each) to two separate 5.0 L flask containing water (1.5 L) and ice cubes (1.5 L, used to cool the water instead of an external bath in addition to having more water in the flask). Upon stirring, solids crashed out and there was also a foam at the top. This crude mixture was filtered and the solids were washed with water, then dried under high vacuum for about 2-3 days to afford 2-amino-6-chloro-5-(trifluoromethyl)pyrimidin-4-ol (34.86 g, 74%) as an off-white solid. ESI-MS m/z calc. 212.9917, found 214.1 (M+1)⁺; Retention time: 1.33 minutes (LC method C).

### Step 2: N′-[4,6-Dichloro-5-(trifluoromethyl)pyrimidin-2-yl]-N,N-dimethyl-formamidine

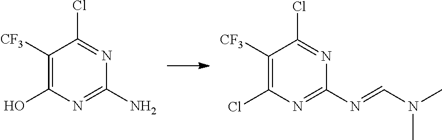 [SMILES: Nc1nc(O)c(C(F)(F)F)c(Cl)n1 ; CN(C)/C=N/c1nc(Cl)c(C(F)(F)F)c(Cl)n1]

Oxalyl chloride (124.70 g, 86 mL, 982.46 mmol) was slowly added to a solution of dimethylformamide (71.440 g, 76 mL, 977.37 mmol) in chloroform (1.25 L) and the solution was stirred at room temperature for 30 minutes. 2-Amino-6-chloro-5-(trifluoromethyl)pyrimidin-4-ol (34.86 g, 163.24 mmol) was added then the reaction mixture was heated at 60° C. for 2 hours. Once cooled to room temperature, the reaction mixture was diluted with saturated sodium bicarbonate solution (2 L) and stirred vigorously for 15 minutes. A 25% sodium hydroxide solution (180 mL) was added to reach pH ˜8-9. The layers were separated and aqueous layer was extracted with dichloromethane (2×500 mL). The organic layers were combined, dried over sodium sulfate and concentrated under reduced pressure to afford N′-[4,6-dichloro-5-(trifluoromethyl)pyrimidin-2-yl]-N,N-dimethyl-formamidine (126.7 g, 270%) as a brown oil. ESI-MS m/z calc. 286, found 287.1 (M+1)⁺; Retention time: 1.82 minutes (LC method C).

### Step 3: 4,6-Dichloro-5-(trifluoromethyl)pyrimidin-2-amine

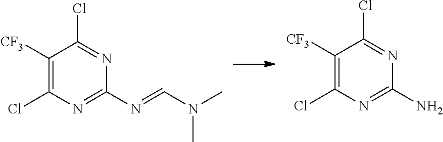 [SMILES: CN(C)/C=N/c1nc(Cl)c(C(F)(F)F)c(Cl)n1 ; Nc1nc(Cl)c(C(F)(F)F)c(Cl)n1]

Hydrochloric acid (85 mL of 12 M, 1.0200 mol) was added to a solution of N′-[4,6-dichloro-5-(trifluoromethyl)pyrimidin-2-yl]-N,N-dimethyl-formamidine (46.86 g, 163.24 mmol) in isopropanol (950 mL) and the mixture was stirred at 50° C. for 90 minutes. The solution was concentrated under reduced pressure and ethyl acetate (800 mL) was added. The organic phase was washed with water (2×300 mL) and brine (300 mL), dried with anhydrous sodium sulfate and concentrated under reduced pressure. The residue was purified on silica gel chromatography (2 successive column) eluting from 0 to 40% ethyl acetate in heptanes then triturated in a mix of ethyl acetate and heptanes (˜1:19), filtered and dried to afford 4,6-dichloro-5-(trifluoromethyl)pyrimidin-2-amine (24.4 g, 63%) as a white solid. ¹H NMR (300 MHz, DMSO-d₆) ppm 8.30 (br. s., 2H). ¹⁹F NMR (282 MHz, DMSO-d₆) ppm −53.4 (s, 3F). ESI-MS m/z calc. 230.9578, found 232.0 (M+1)⁺; Retention time: 2.51 minutes (LC method B).

### Step 4: N-[4,6-Dichloro-5-(trifluoromethyl)pyrimidin-2-yl]-1-methyl-pyrazole-4-sulfonamide

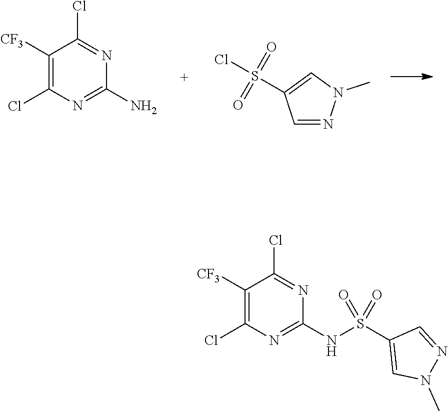

To a solution of 4,6-dichloro-5-(trifluoromethyl)pyrimidin-2-amine (1 g, 4.311 mmol) in DMF (17 mL) at 0° C. was added sodium hydride (690 mg of 60% w/w, 17.25 mmol). The reaction was allowed to warm to 23° C. over 15 minutes and then recooled to 0° C. before introducing 1-methylpyrazole-4-sulfonyl chloride (1.56 g, 8.637 mmol). The reaction was stirred for 15 minutes and then quenched with acetic acid (3.7 mL, 65.06 mmol). The crude solution was partition between water and ethyl acetate. The organic layer was separated, and the aqueous layer was further extracted with ethyl acetate (4×). The combined organics were washed with brine (2×) and concentrated in vacuo. The crude residue was triturated with a small quantity of acetone and filtered to afford N-[4,6-dichloro-5-(trifluoromethyl)pyrimidin-2-yl]-1-methyl-pyrazole-4-sulfonamide (1.47 g, 90%) as a light yellow solid which was used without further purification. ESI-MS m/z calc. 374.95712, found 376.03 (M+1)⁺; Retention time: 0.55 minutes; LC method D.

### Step 5: 3-(4-Methylpiperazin-1-yl)phenol

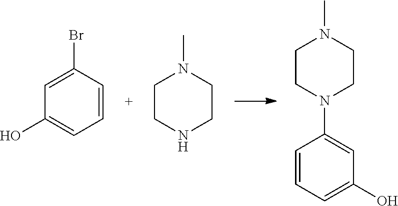

In a glass vial were 3-bromophenol (51.9 mg, 0.300 mmol), [2-(2-aminoethyl)phenyl]-chloro-palladium;ditert-butyl-[2-(2,4,6-triisopropylphenyl)phenyl]phosphane (XPhos Pd G1)(26.5 mg, 0.0386 mmol), dioxane (800 μL), 1-methylpiperazine (100 μL), and sodium tert-butoxide (60.4 mg, 0.628 mmol) and the mixture was sparged under nitrogen for 5 minutes and then stirred at 35° C. for 30 minutes. The solution was filtered and the resulting residues dissolved in 1.2 mL DMSO/MeOH (1:1), and purified by reverse phase chromatography using a 15 minutes gradient of 100 MeCN in water to 99% MeCN (HCl modifier) to give 3-(4-methylpiperazin-1-yl)phenol (hydrochloride salt) (66.3 mg, 97% ) ESI-MS m/z calc. 192.12627, found 193.29 (M+1)⁺; Retention time: 0.63 minutes. LC method A.

### Step 6: N-[4-chloro-6-[3-(4-methylpiperazin-1-yl)phenoxy]-5-(trifluoromethyl)pyrimidin-2-yl]-1-methyl-pyrazole-4-sulfonamide

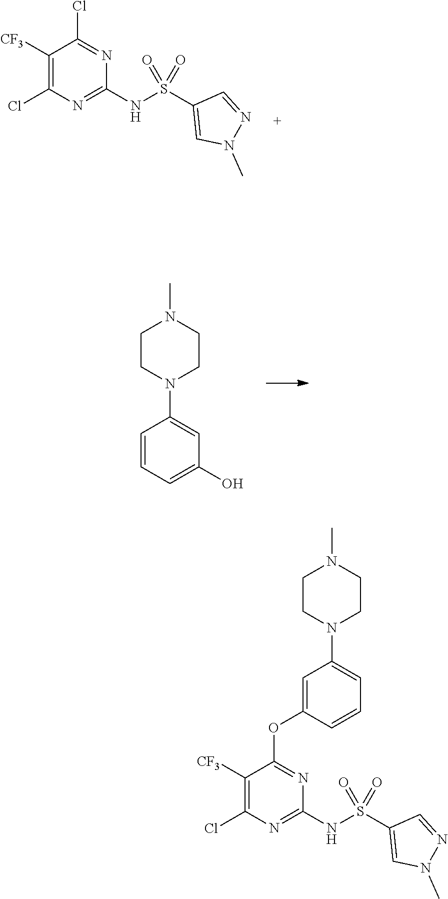 [SMILES: Cn1cc(S(=O)(=O)Nc2nc(Cl)c(C(F)(F)F)c(Cl)n2)cn1 ; CN1CCN(c2cccc(O)c2)CC1 ; CN1CCN(c2cccc(Oc3nc(NS(=O)(=O)c4cnn(C)c4)nc(Cl)c3C(F)(F)F)c2)CC1]

A heterogeneous solution of N-[4,6-dichloro-5-(trifluoromethyl)pyrimidin-2-yl]-1 -methyl-pyrazole-4-sulfonamide (200 mg, 0.5317 mmol), potassium carbonate (220 mg, 1.592 mmol), and 3-(4-methylpiperazin-1-yl)phenol (100 mg, 0.5201 mmol) in NMP (1 mL) was heated in a sealed vial to 60° C. for 16 hours. The crude solution was acidified with acetic acid (600 μL, 10.55 mmol), diluted with water (0.20 mL), and filtered through a 0.2 μm syringe filter. The sample was purified by reverse phase HPLC (waters Sunfire C₁₈ column (100×50 mm, 10 μm particle size), gradient: 1-99% acetonitrile in water (5 mM HCl) over 15.0 minutes) to afford N-[4-chloro-6-[3-(4-methylpiperazin-1-yl)phenoxy]-5-(trifluoromethyl)pyrimidin-2-yl]-1-methyl-pyrazole-4-sulfonamide (30 mg, 11%) as a white solid. ESI-MS m/z calc. 531.10675, found 532.17 (M+1)⁺; Retention time: 0.52 minutes; LC method D.

### Step 7: 1-Methyl-N-[4-[3-(4-methylpiperazin-1-yl)phenoxy]-6-(o-tolyl)-5-(trifluoromethyl)pyrimidin-2-yl]pyrazole-4-sulfonamide (Compound 487)

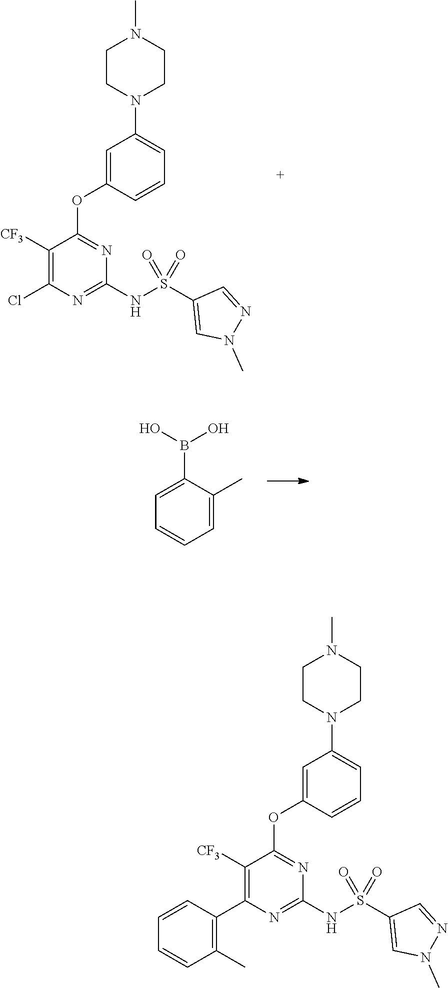

A heterogeneous solution of N-[4-chloro-6-[3-(4-methylpiperazin-1-yl)phenoxy]-5-(trifluoromethyl)pyrimidin-2-yl]-1-methyl-pyrazole-4-sulfonamide (27 mg, 0.05076 mmol), o-tolylboronic acid (13.8 mg, 0.1015 mmol), tetrakis(triphenylphosphine)palladium(0) (11.7 mg, 0.01012 mmol), and potassium carbonate (21.1 mg, 0.1527 mmol) in dioxane (200 μL) and water (40 μL) was microwaved at 125° C. for 20 minutes. The crude solution was acidified with acetic acid (60 μL, 1.055 mmol), diluted with DMSO (0.20 ml), and filtered through a 0.20 m syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford 1-methyl-N-[4-[3-(4-methylpiperazin-1-yl)phenoxy]-6-(o-tolyl)-5-(trifluoromethyl)pyrimidin-2-yl]pyrazole-4-sulfonamide (hydrochloride salt) (10.7 mg, 34%) as a white solid. ESI-MS m/z calc. 587.1926, found 588.28 (M+1)⁺; Retention time: 1.45 minutes (LC method A).

### Example 177: Preparation of Compound 488

### Step 1: N-[4-chloro-6-(2-methylphenoxy)-5-(trifluoromethyl)pyrimidin-2-yl]-1 -methyl-pyrazole-4-sulfonamide

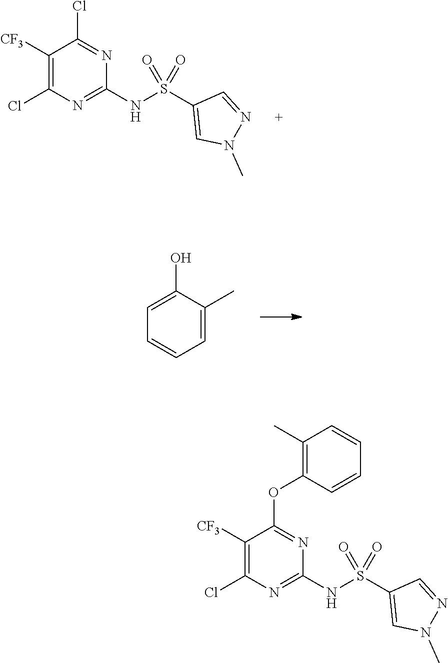

A heterogeneous mixture of N-[4,6-dichloro-5-(trifluoromethyl)pyrimidin-2-yl]-1 -methyl-pyrazole-4-sulfonamide (60 mg, 0.1579 mmol), o-cresol (17 mg, 0.1572 mmol), and potassium carbonate (44 mg, 0.3184 mmol) in NMP (315 μL) was heated in a sealed vial to 120°C. for 16 hours. The reaction mixture was acidified with acetic acid (135 μL, 2.374 mmol) and further diluted with DMSO (0.5 mL). The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-chloro-6-(2-methylphenoxy)-5-(trifluoromethyl)pyrimidin-2-yl]-1-methyl-pyrazole-4-sulfonamide (30 mg, 42%) as a white solid. ESI-MS m/z calc. 447.03796, found 448.12 (M+1)⁺; Retention time: 0.69 minutes; LC method D.

### Step 2: 1-methyl-N-[4-(2-methylphenoxy)-6-(o-tolyl)-5-(trifluoromethyl)pyrimidin-2-yl]pyrazole-4-sulfonamide

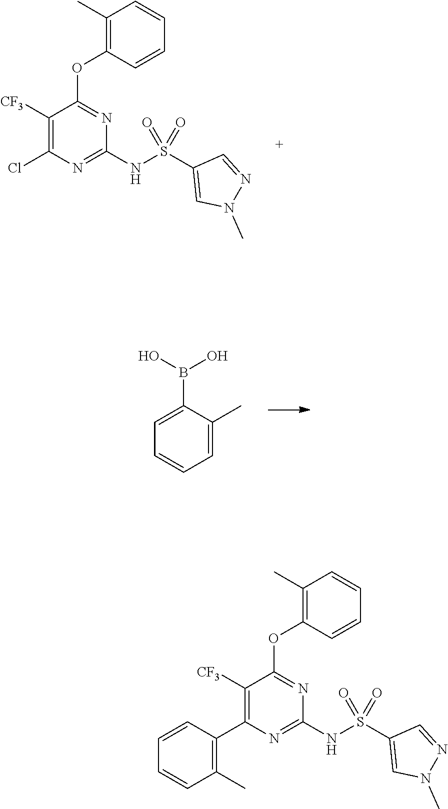

A heterogeneous solution consisting of N-[4-chloro-6-(2-methylphenoxy)-5-(trifluoromethyl)pyrimidin-2-yl]-1-methyl-pyrazole-4-sulfonamide (14.5 mg, 0.03238 mmol), o-tolylboronic acid (8.8 mg, 0.06473 mmol), potassium carbonate (13.4 mg, 0.09696 mmol), and bis(triphenylphosphine)palladium(II)dichloride (2.3 mg, 0.003277 mmol) in dioxane (135 μL) and water (27 μL) was microwaved in a sealed vial to 120° C. for 15 minutes. The reaction was diluted with DMSO (0.5 mL) and acidified with acetic acid (15 μL, 0.2638 mmol). The crude sample was purified by reverse phase HPLC (Phenomenex Luna C₁₈ column (75×30 mm, 5 m particle size), gradient: 1-99% acetonitrile in water (5 mM HCl) over 15.0 minutes) to afford 1 -methyl-N-[4-(2-methylphenoxy)-6-(o-tolyl)-5-(trifluoromethyl)pyrimidin-2-yl]pyrazole-4-sulfonamide (9.8 mg, 60%) as a white solid. ESI-MS m/z calc. 503.1239, found 504.27 (M+1)⁺; Retention time: 2.01 minutes; LC method A.

### Example 178: Preparation of Compound 489

### Step 1: N-[4-chloro-6-[4-(4-methylpiperazin-1-yl)phenoxy]-5-(trifluoromethyl)pyrimidin-2-yl]-1-methyl-pyrazole-4-sulfonamide

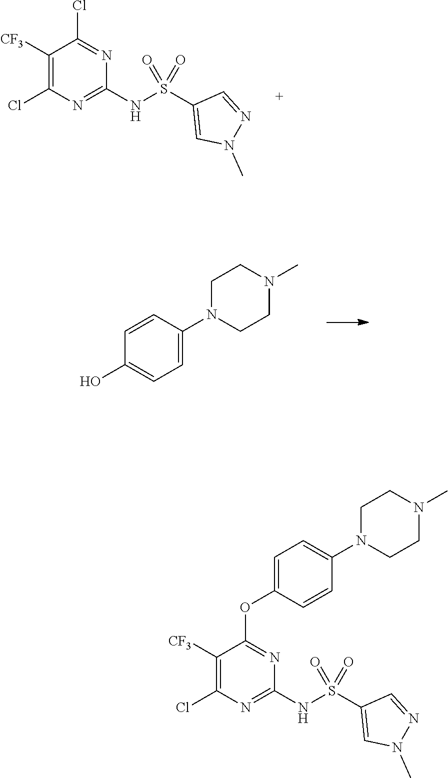 [SMILES: Cn1cc(S(=O)(=O)Nc2nc(Cl)c(C(F)(F)F)c(Cl)n2)cn1 ; CN1CCN(c2ccc(O)cc2)CC1 ; CN1CCN(c2ccc(Oc3nc(NS(=O)(=O)c4cnn(C)c4)nc(Cl)c3C(F)(F)F)cc2)CC1]

A heterogeneous solution of N-[4,6-dichloro-5-(trifluoromethyl)pyrimidin-2-yl]-1 -methyl-pyrazole-4-sulfonamide (247.0 mg, 0.6567 mmol), potassium carbonate (272 mg, 1.968 mmol), and 4-(4-methylpiperazin-1-yl)phenol (126 mg, 0.6554 mmol) in NMP (1.3 mL) was heated to 60° C. for 16 hours in a sealed vial. The reaction was acidified with hydrochloric acid (650 μL of 37% w/v, 6.596 mmol) and diluted with water (˜1 mL). The sample was purified by reverse phase HPLC (Waters Sunfire C₁₈ column (100×50 mm, 10 m particle size), gradient: 1-99% acetonitrile in water (5 mM HCl) over 15.0 minutes) to afford N-[4-chloro-6-[4-(4-methylpiperazin-1-yl)phenoxy]-5-(trifluoromethyl)pyrimidin-2-yl]-1-methyl-pyrazole-4-sulfonamide (70 mg, 20%) as a white solid. ESI-MS m/z calc. 531.10675, found 531.9 (M+1)⁺; Retention time: 0.47 minutes; LC method D.

### Step 2: N-[4-(2,6-Dimethylphenyl)-6-[4-(4-methylpiperazin-1-yl)phenoxy]-5-(trifluoromethyl)pyrimidin-2-yl]-1-methyl-pyrazole-4-sulfonamide

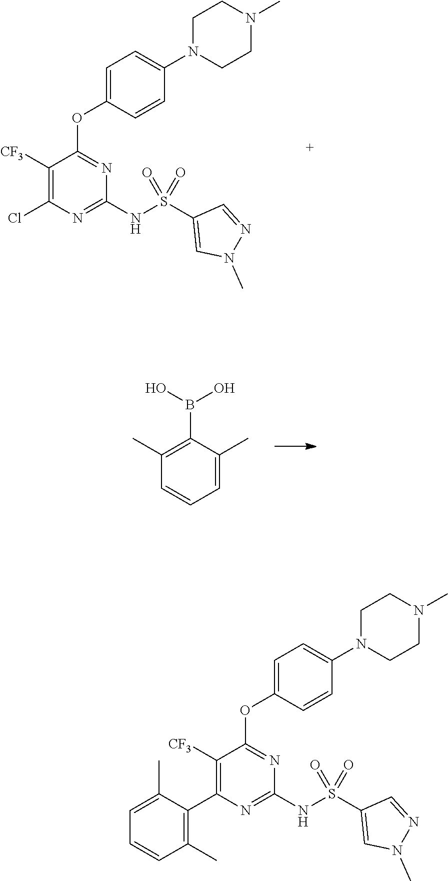

A heterogeneous solution of N-[4-chloro-6-[4-(4-methylpiperazin-1-yl)phenoxy]-5-(trifluoromethyl)pyrimidin-2-yl]-1-methyl-pyrazole-4-sulfonamide (70 mg, 0.1316 mmol), potassium carbonate (73 mg, 0.5282 mmol), (2,6-dimethylphenyl)boronic acid (42 mg, 0.2800 mmol), and tetrakis(triphenylphosphine)palladium(0) (30 mg, 0.02596 mmol) in dioxane (550 μL) and water (110 μL) was microwaved in a sealed vial to 120° C. for 15 minutes. The reaction mixture was acidified with acetic acid (150 μL, 2.638 mmol) and filtered. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-(2,6-dimethylphenyl)-6-[4-(4-methylpiperazin-1-yl)phenoxy]-5-(trifluoromethyl)pyrimidin-2-yl]-1 -methyl-pyrazole-4-sulfonamide (hydrochloride salt) (1.3 mg, 2%) as a white solid. ESI-MS m z calc. 601.2083, found 602.1 (M+1)⁺; Retention time: 1.37 minutes; LC method A.

### Example 179: Preparation of Compound 490

### Step 1: 2-chloro-3-(4-methylpiperazin-1-yl)phenol

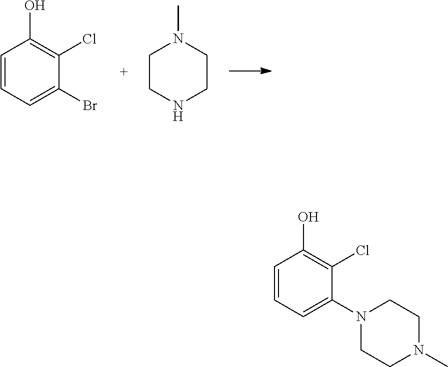

A heterogeneous mixture of 3-bromo-2-chloro-phenol (4.20 g, 20.25 mmol), 1-methylpiperazine (21.5 g, 214.7 mmol), Chloro(2-di-t-butylphosphino-2′, 4′, 6′-tri-i-propyl-1,1′-biphenyl)[2-(2-aminoethyl)phenyl] palladium(II) [t-BuXPhos Palladacycle Gen. 1] (2.1 g, 3.058 mmol), and potassium tert-butoxide (4.8 g, 42.78 mmol) in dioxane (120 mL) was sonicated for 15 minutes and heated in a sealed vessel at 50° C. for 16 hours. The reaction mixture was acidified using acetic acid (3.4 mL, 59.79 mmol) then partitioned between DCM (100 mL) and water (100 mL). The organic layer was separated, and the aqueous layer was further extracted with DCM (4×). The combined organics were washed once with brine, dried using magnesium sulfate, filtered, and concentrated in vacuo. The crude residue was separated by flash column chromatography on silica gel (gradient: 1 to 10% methanol in dichloromethane) to afford 2-chloro-3-(4-methylpiperazin-1-yl)phenol (3.86 g, 84%) as a yellow solid. ESI-MS m/z calc. 226.0873, found 227.12 (M+1)⁺; Retention time: 0.24 minutes; LC method D.

### Step 2: N-[4-chloro-6-[2-chloro-3-(4-methylpiperazin-1-yl)phenoxy]-5-(trifluoromethyl)pyrimidin-2-yl]-1-methyl-pyrazole-4-sulfonamide

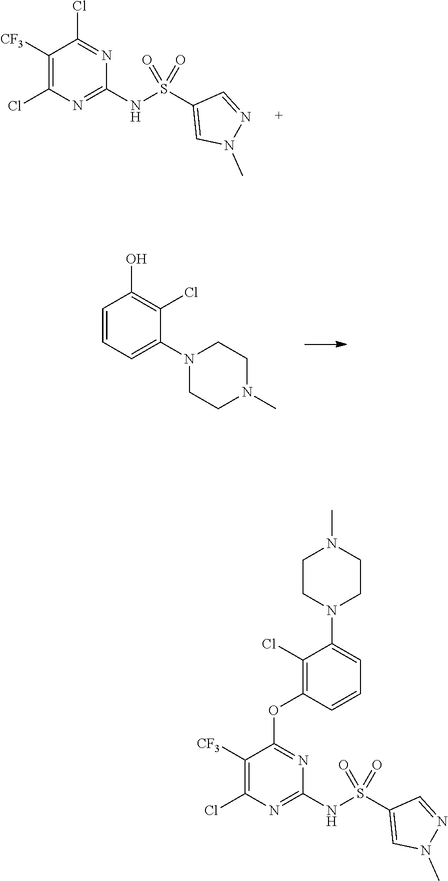 [SMILES: Cn1cc(S(=O)(=O)Nc2nc(Cl)c(C(F)(F)F)c(Cl)n2)cn1 ; CN1CCN(c2cccc(O)c2Cl)CC1 ; CN1CCN(c2cccc(Oc3nc(NS(=O)(=O)c4cnn(C)c4)nc(Cl)c3C(F)(F)F)c2Cl)CC1]

A heterogeneous solution of N-[4,6-dichloro-5-(trifluoromethyl)pyrimidin-2-yl]-1 -methyl-pyrazole-4-sulfonamide (200 mg, 0.5317 mmol), 2-chloro-3-(4-methylpiperazin-1-yl)phenol (approximately 120.5 mg, 0.5317 mmol), and potassium carbonate (approximately 220.4 mg, 1.595 mmol) in NMP (1 mL) was heated to 60° C. for 16 hours. The solution was acidified with acetic acid (approximately 638.4 mg, 604.5 μL, 10.63 mmol) and filtered through a 0.45 m PTFE syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-chloro-6-[2-chloro-3-(4-methylpiperazin-1-yl)phenoxy]-5-(trifluoromethyl)pyrimidin-2-yl]-1-methyl-pyrazole-4-sulfonamide (74 mg, 25%) as a white solid. ESI-MS m/z calc. 565.06775, found 566.17 (M+1)⁺; Retention time: 0.52 minutes; LC method D.

### Step 3: N-[4-[2-chloro-3-(4-methylpiperazin-1-yl)phenoxy]-6-(o-tolyl)-5-(trifluoromethyl)pyrimidin-2-yl]-1-methyl-pyrazole-4-sulfonamide

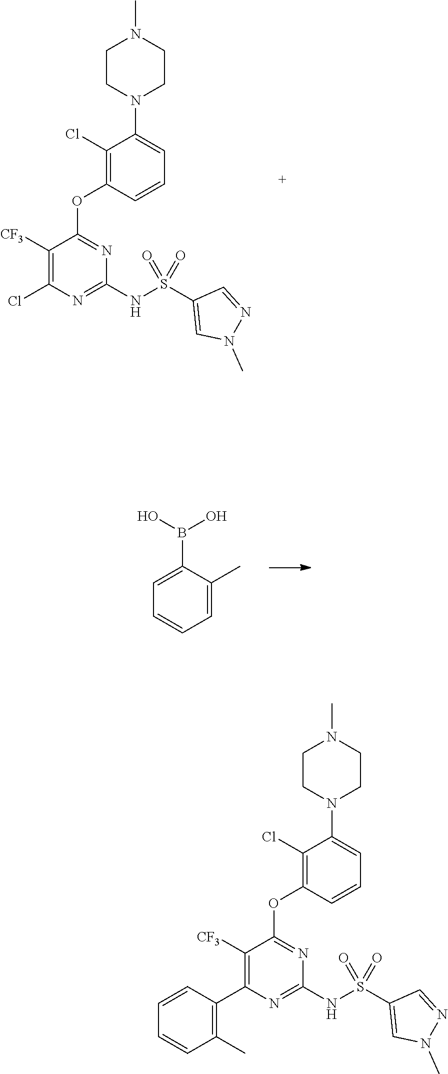

A heterogeneous solution of N-[4-chloro-6-[2-chloro-3-(4-methylpiperazin-1-yl)phenoxy]-5-(trifluoromethyl)pyrimidin-2-yl]-1-methyl-pyrazole-4-sulfonamide (28.0 mg, 0.04944 mmol), o-tolylboronic acid (13.4 mg, 0.09856 mmol), potassium carbonate (27.3 mg, 0.1975 mmol), and tetrakis(triphenylphosphine)palladium(0) (11.4 mg, 0.009865 mmol) in dioxane (200 μL) and water (40 μL) was microwaved at 125° C. for 20 minutes. The reaction mixture was acidified with acetic acid (60 μL, 1.055 mmol), diluted with water (0.20 ml) and DMSO (0.20 ml) and subsequently filtered through a 0.20 m syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-[2-chloro-3-(4-methylpiperazin-1-yl)phenoxy]-6-(o-tolyl)-5-(trifluoromethyl)pyrimidin-2-yl]-1 -methyl-pyrazole-4-sulfonamide (hydrochloride salt) (14 mg, 43%) as a white solid. ESI-MS m z calc. 621.1537, found 622.27 (M+1)⁺; Retention time: 1.52 minutes; LC method A.

### Example 180: Preparation of Compound 491

### Step 1: N-[4-(4-chlorophenoxy)-6-(2-isopentylphenyl)-5-(trifluoromethyl)pyrimidin-2-yl]-1-methyl-pyrazole-4-sulfonamide

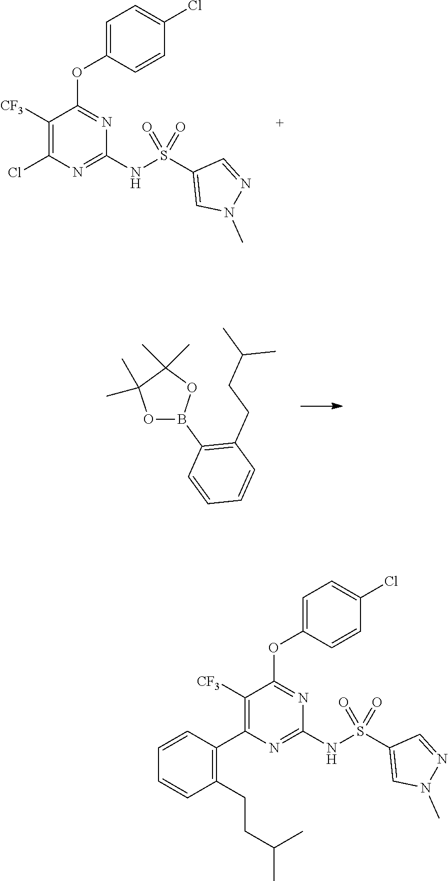 [SMILES: Cn1cc(S(=O)(=O)Nc2nc(Cl)c(C(F)(F)F)c(Oc3ccc(Cl)cc3)n2)cn1 ; CC(C)CCc1ccccc1B1OC(C)(C)C(C)(C)O1 ; CC(C)CCc1ccccc1-c1nc(NS(=O)(=O)c2cnn(C)c2)nc(Oc2ccc(Cl)cc2)c1C(F)(F)F]

A heterogeneous solution of Pd(PPh₃)₄ (305.5 mg, 0.2644 mmol), potassium carbonate (731 mg, 5.289 mmol), N-[4-chloro-6-(4-chlorophenoxy)-5-(trifluoromethyl)pyrimidin-2-yl]-1-methyl-pyrazole-4-sulfonamide (680.0 mg, 1.322 mmol), and 2-(2-isopentylphenyl)-4,4,5,5-tetramethyl-1,3,2-dioxaborolane (435 mg, 1.586 mmol) in dioxane (5.50 mL) and water (1.10 mL) was microwaved to 100° C. for 20 minutes. The crude solution was partitioned between ethyl acetate and a hydrochloric acid solution (1 N aqueous). The organic layer was separated, and the aqueous layer was further extracted with ethyl acetate (3×). The combined organics were dried over magnesium sulfate, filtered, and concentrated in vacuo. The crude residue was separated by flash column chromatography on silica gel (10 to 100% ethyl acetate in hexanes) to afford N-[4-(4-chlorophenoxy)-6-(2-isopentylphenyl)-5-(trifluoromethyl)pyrimidin-2-yl]-1-methyl-pyrazole-4-sulfonamide (310 mg, 37%) as a white solid. ESI-MS m/z calc. 579.1319, found 580.21 (M+1)⁺; Retention time: 0.91 minutes; LC method D.

### Step 2: N-[4-(2-isopentylphenyl)-6-[4-(4-methylpiperazin-1-yl)phenoxy]-5-(trifluoromethyl)pyrimidin-2-yl]-1-methyl-pyrazole-4-sulfonamide

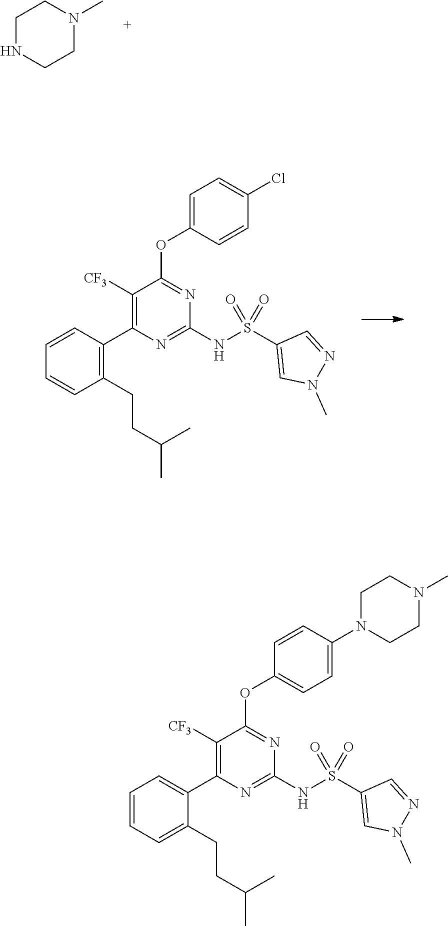

A heterogeneous solution of 1-methylpiperazine (approximately 2.829 mg, 0.02824 mmol), N-[4-(4-chlorophenoxy)-6-(2-isopentylphenyl)-5-(trifluoromethyl)pyrimidin-2-yl]-1 -methyl-pyrazole-4-sulfonamide (15.0 mg, 0.02353 mmol), potassium tert-butoxide (approximately 6.600 mg, 0.05882 mmol), and Chloro(2-di-t-butylphosphino-2′, 4′, 6′-tri-i-propyl-1,1′-biphenyl)[2-(2-aminoethyl)phenyl] palladium(II)[t-BuXPhos Palladacycle Gen. 1](approximately 1.616 mg, 0.002353 mmol) in dioxane (117.6 μL) was heated to 100° C. for 7 minutes. The reaction was diluted with DMSO. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-(2-isopentylphenyl)-6-[4-(4-methylpiperazin-1-yl)phenoxy]-5-(trifluoromethyl)pyrimidin-2-yl]-1-methyl-pyrazole-4-sulfonamide (hydrochloride salt) (1.5 mg, 8%) as a white solid. ESI-MS m/z calc. 643.25525, found 644.42 (M+1)⁺; Retention time: 1.83 minutes; LC method A.

### Example 181: Preparation of Compound 492

### Step 1: N-[4-(2-isopentylphenyl)-6-(4-morpholinophenoxy)-5-(trifluoromethyl)pyrimidin-2-yl]-1-methyl-pyrazole-4-sulfonamide

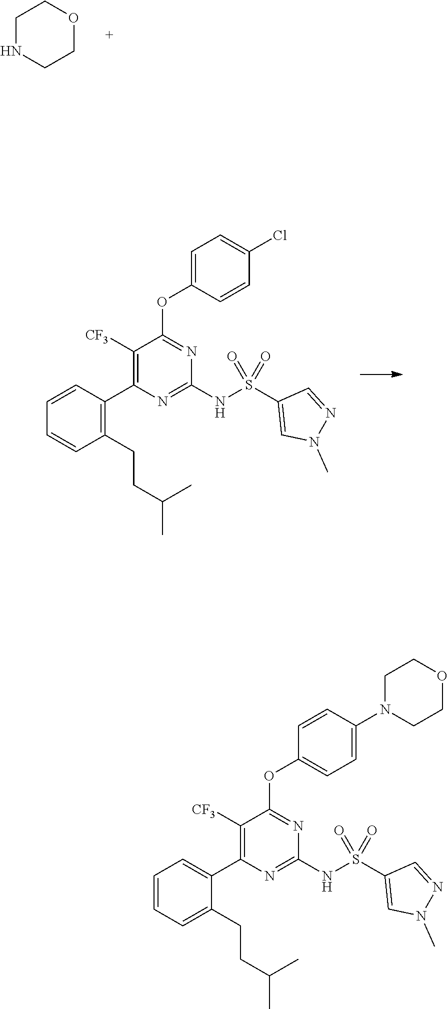 [SMILES: C1COCCN1 ; CC(C)CCc1ccccc1-c1nc(NS(=O)(=O)c2cnn(C)c2)nc(Oc2ccc(Cl)cc2)c1C(F)(F)F ; CC(C)CCc1ccccc1-c1nc(NS(=O)(=O)c2cnn(C)c2)nc(Oc2ccc(N3CCOCC3)cc2)c1C(F)(F)F]

A heterogeneous solution of morpholine (approximately 2.460 mg, 2.462 μL, 0.02824 mmol), N-[4-(4-chlorophenoxy)-6-(2-isopentylphenyl)-5-(trifluoromethyl)pyrimidin-2-yl]-1 -methyl-pyrazole-4-sulfonamide (15.0 mg, 0.02353 mmol), potassium tert-butoxide (approximately 6.600 mg, 0.05882 mmol), and Chloro(2-di-t-butylphosphino-2′, 4′, 6′-tri-i-propyl-1,1′-biphenyl)[2-(2-aminoethyl)phenyl] palladium(II)[t-BuXPhos Palladacycle Gen. 1](approximately 1.616 mg, 0.002353 mmol) in dioxane (117.6 μL) was heated to 100° C. for 7 minutes. The reaction was diluted with DMSO. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-(2-isopentylphenyl)-6-(4-morpholinophenoxy)-5-(trifluoromethyl)pyrimidin-2-yl]-1-methyl-pyrazole-4-sulfonamide (7.6 mg, 51%) as a white solid. ESI-MS m/z calc. 630.22363, found 631.24 (M+1)⁺; Retention time: 2.24 minutes; LC method A.

### Example 182: Preparation of Compound 493

### Step 1: N-[4-chloro-6-[4-(1-methyl-4-piperidyl)phenoxy]-5-(trifluoromethyl)pyrimidin-2-yl]-1-methyl-pyrazole-4-sulfonamide

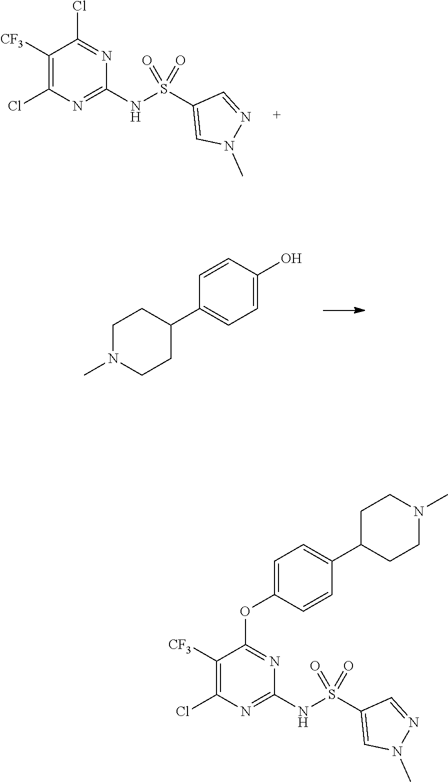

A mixture of N-[4,6-dichloro-5-(trifluoromethyl)pyrimidin-2-yl]-1-methyl-pyrazole-4-sulfonamide (244.5 mg, 0.6500 mmol), 4-(1-methyl-4-piperidyl)phenol (acetate salt) (108.9 mg, 0.4333 mmol), and Cs₂CO₃ (699.7 mg, 2.148 mmol) in NMP (1.5 mL) was heated at 110° C. for 15 minutes and then cooled to room temperature. The solution was filtered and the filtrate was dissolved in 0.8 mL MeOH, and purified by reverse phase chromatography using a 15 minutes gradient of 1% MeCN in water to 99% MeCN (HCl modifier) to give N-[4-chloro-6-[4-(1-methyl-4-piperidyl)phenoxy]-5-(trifluoromethyl)pyrimidin-2-yl]-1-methyl-pyrazole-4-sulfonamide (127.6 mg, 55%) ESI-MS m/z calc. 530.11145, found 531.15 (M+1)⁺; Retention time: 1.31 minutes. LC method A.

### Step 2: N-[4-(2-isobutylphenyl)-6-[4-(1-methyl-4-piperidyl)phenoxy]-5-(trifluoromethyl)pyrimidin-2-yl]-1-methyl-pyrazole-4-sulfonamide

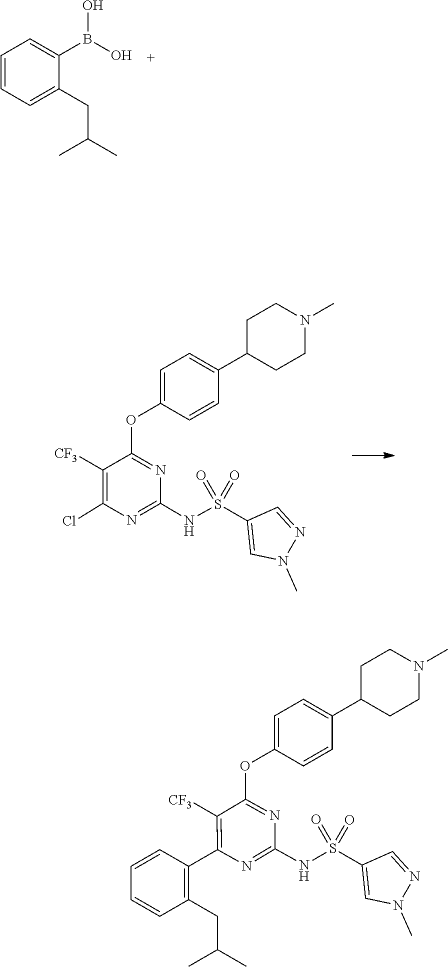 [SMILES: CC(C)Cc1ccccc1B(O)O ; CN1CCC(c2ccc(Oc3nc(NS(=O)(=O)c4cnn(C)c4)nc(Cl)c3C(F)(F)F)cc2)CC1 ; CC(C)Cc1ccccc1-c1nc(NS(=O)(=O)c2cnn(C)c2)nc(Oc2ccc(C3CCN(C)CC3)cc2)c1C(F)(F)F]

A heterogeneous solution of (2-isobutylphenyl)boronic acid (approximately 33.19 mg, 0.1864 mmol), N-[4-chloro-6-[4-(1-methyl-4-piperidyl)phenoxy]-5-(trifluoromethyl)pyrimidin-2-yl]-1-methyl-pyrazole-4-sulfonamide (33 mg, 0.06215 mmol), potassium carbonate (approximately 34.36 mg, 0.2486 mmol), and tetrakis(triphenylphosphine)palladium(0) (approximately 14.36 mg, 0.01243 mmol) in dioxane (259.0 μL) and water (51.79 μL) was microwaved to 120° C. for 20 minutes. The reaction mixture was diluted with water (0.10 mL), acidified with acetic acid (approximately 37.32 mg, 35.34 μL, 0.6215 mmol), and filtered through a 0.45 μM PTFE syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-(2-isobutylphenyl)-6-[4-(1-methyl-4-piperidyl)phenoxy]-5-(trifluoromethyl)pyrimidin-2-yl]-1-methyl-pyrazole-4-sulfonamide (hydrochloride salt) as a white solid (5.6 mg, 13.5%). ESI-MS m/z calc. 628.2443, found 629.42 (M+1)⁺; Retention time: 1.59 minutes; LC method A.

### Example 183: Preparation of Compound 494

### Step 1: N-[4-(2-isopropylphenyl)-6-[4-(1-methyl-4-piperidyl)phenoxy]-5-(trifluoromethyl)pyrimidin-2-yl]-1-methyl-pyrazole-4-sulfonamide

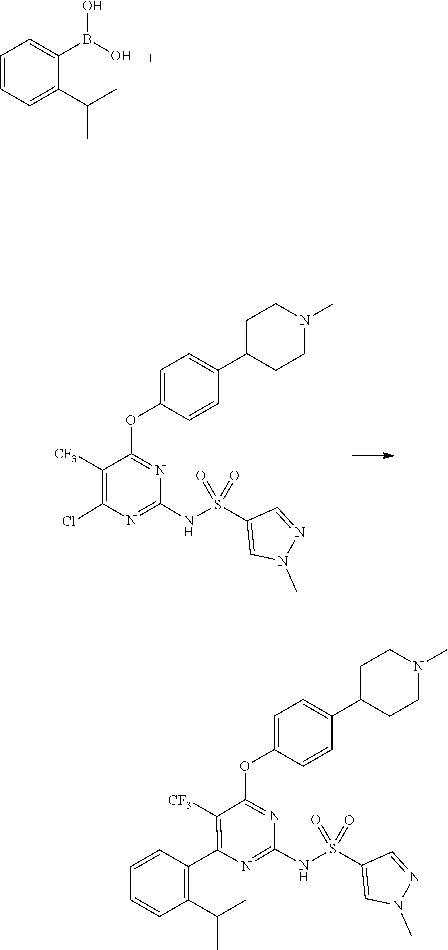 [SMILES: CC(C)c1ccccc1B(O)O ; CN1CCC(c2ccc(Oc3nc(NS(=O)(=O)c4cnn(C)c4)nc(Cl)c3C(F)(F)F)cc2)CC1 ; CC(C)c1ccccc1-c1nc(NS(=O)(=O)c2cnn(C)c2)nc(Oc2ccc(C3CCN(C)CC3)cc2)c1C(F)(F)F]

A heterogeneous solution of (2-isopropylphenyl)boronic acid (approximately 30.57 mg, 0.1864 mmol), N-[4-chloro-6-[4-(1-methyl-4-piperidyl)phenoxy]-5-(trifluoromethyl)pyrimidin-2-yl]-1-methyl-pyrazole-4-sulfonamide (33 mg, 0.06215 mmol), potassium carbonate (approximately 34.36 mg, 0.2486 mmol), and tetrakis(triphenylphosphine)palladium(0) (approximately 14.36 mg, 0.01243 mmol) in dioxane (259.0 μL) and water (51.79 μL) was microwaved to 120° C. for 20 minutes. The reaction mixture was diluted with water (0.10 mL), acifidified with acetic acid (approximately 37.32 mg, 35.34 μL, 0.6215 mmol), and filtered through a 0.45 μM PTFE syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-(2-isopropylphenyl)-6-[4-(1-methyl-4-piperidyl)phenoxy]-5-(trifluoromethyl)pyrimidin-2-yl]-1 -methyl-pyrazole-4-sulfonamide (hydrochloride salt) as a white solid (3.9 mg, 10%). ESI-MS m z calc. 614.2287, found 615.34 (M+1)⁺; Retention time: 1.52 minutes; LC method A.

### Example 184: Preparation of Compound 495

### Step 1: 1-methyl-N-[4-[4-(1-methyl-4-piperidyl)phenoxy]-6-(o-tolyl)-5-(trifluoromethyl)pyrimidin-2-yl]pyrazole-4-sulfonamide

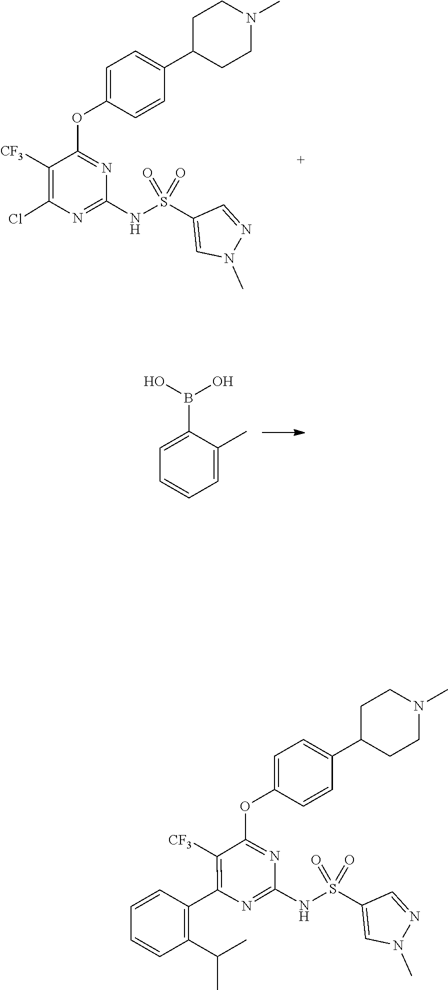

A dioxane (0.8 mL) mixture of N-[4-chloro-6-[4-(1-methyl-4-piperidyl)phenoxy]-5-(trifluoromethyl)pyrimidin-2-yl]-1-methyl-pyrazole-4-sulfonamide (formic acid salt) (53.3 mg, 0.1004 mmol), o-tolylboronic acid (39.2 mg, 0.2883 mmol), Pd(PPh₃)₄ (22.7 mg, 0.01964 mmol), and K₂CO₃ (200 μL of 2 M, 0.4000 mmol) was sparged with nitrogen for 1 minute and then microwaved at 120° C. for 20 minutes. The solution was filtered and the filtrate dissolved in 0.8 mL MeOH, and purified by reverse phase chromatography using a 15 minutes gradient of 20% MeCN in water to 80% MeCN (HCl modifier) to give 1-methyl-N-[4-[4-(1-methyl-4-piperidyl)phenoxy]-6-(o-tolyl)-5-(trifluoromethyl)pyrimidin-2-yl]pyrazole-4-sulfonamide (hydrochloride salt) (20.5 mg, 33%) ¹H NMR (400 MHz, DMSO-d₆) δ 10.38 (s, 1H), 7.49 (s, 1H), 7.46-7.41 (m, 2H), 7.41-7.24 (m, 5H), 7.19 (dd, J=7.6, 1.3 Hz, 1H), 6.96 (s, 1H), 3.73 (s, 3H), 3.50 (d, J=12.0 Hz, 2H), 3.15-2.99 (m, 2H), 2.98-2.84 (m, 1H), 2.77 (d, J=4.6 Hz, 3H), 2.12 (s, 3H), 2.02 (h, J=4.0 Hz, 4H). ESI-MS m/z calc. 586.1974, found 587.33 (M+1)⁺; Retention time: 1.45 minutes. LC method A.

### Example 185: Preparation of Compound 496

### Step 1: N-[4-(2-isopentylphenyl)-6-[4-(1-methyl-4-piperidyl)phenoxy]-5-(trifluoromethyl)pyrimidin-2-yl]-1-methyl-pyrazole-4-sulfonamide

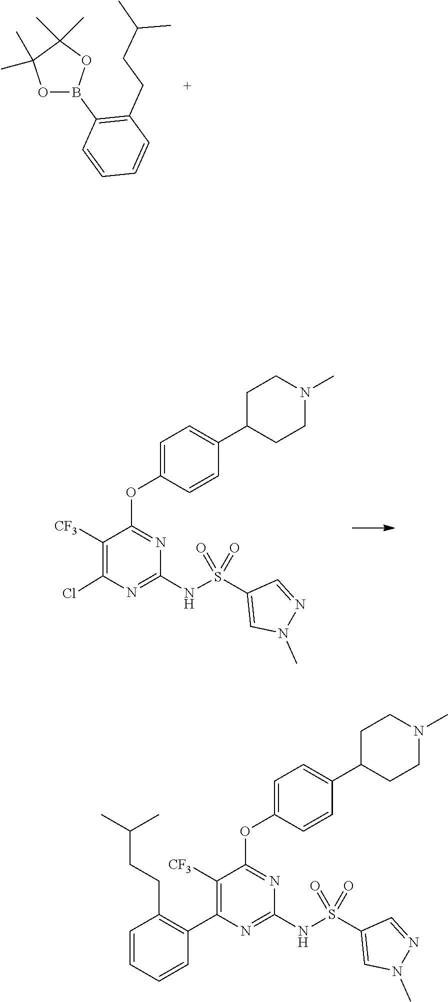 [SMILES: CC(C)CCc1ccccc1B1OC(C)(C)C(C)(C)O1 ; CN1CCC(c2ccc(Oc3nc(NS(=O)(=O)c4cnn(C)c4)nc(Cl)c3C(F)(F)F)cc2)CC1 ; CC(C)CCc1ccccc1-c1nc(NS(=O)(=O)c2cnn(C)c2)nc(Oc2ccc(C3CCN(C)CC3)cc2)c1C(F)(F)F]

A dioxane (0.5 mL) solution of 2-(2-isopentylphenyl)-4,4,5,5-tetramethyl-1,3,2-dioxaborolane (approximately 15.49 mg, 0.05650 mmol), N-[4-chloro-6-[4-(1-methyl-4-piperidyl)phenoxy]-5-(trifluoromethyl)pyrimidin-2-yl]-1-methyl-pyrazole-4-sulfonamide (formic acid salt) (20 mg, 0.03767 mmol), Pd(PPh₃)₄ (approximately 5.802 mg, 0.005021 mmol), and K₂CO₃ (approximately 62.80 μL of 2 M, 0.1256 mmol) was sparged with nitrogen for 30 seconds and then heated at 120° C. for 20 minutes. The mixture was diluted with MeOH (0.5 mL) and the solution was filtered and the filtrate was purified by reverse phase chromatography using a 15 minutes gradient of 20% MeCN in water to 80% MeCN (HCl modifier) to give N-[4-(2-isopentylphenyl)-6-[4-(1-methyl-4-piperidyl)phenoxy]-5-(trifluoromethyl)pyrimidin-2-yl]-1-methyl-pyrazole-4-sulfonamide (hydrochloride salt) (6.3 mg, 25%). ESI-MS m/z calc. 642.26, found 643.2 (M+1)⁺; Retention time: 1.64 minutes; LC method A.

### Example 186: Preparation of Compound 497

### Step 1: N-[4-(2,6-Dimethylphenyl)-6-[4-(1-methyl-4-piperidyl)phenoxy]-5-(trifluoromethyl)pyrimidin-2-yl]-1-methyl-pyrazole-4-sulfonamide

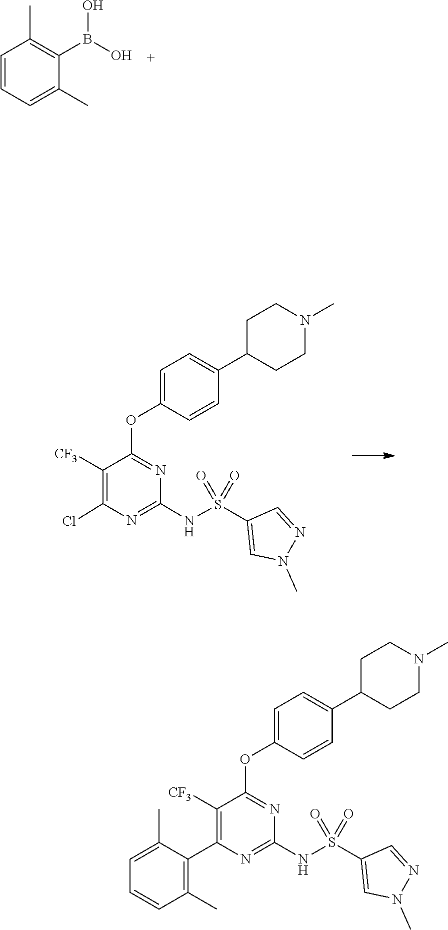 [SMILES: Cc1cccc(C)c1B(O)O ; CN1CCC(c2ccc(Oc3nc(NS(=O)(=O)c4cnn(C)c4)nc(Cl)c3C(F)(F)F)cc2)CC1 ; Cc1cccc(C)c1-c1nc(NS(=O)(=O)c2cnn(C)c2)nc(Oc2ccc(C3CCN(C)CC3)cc2)c1C(F)(F)F]

A heterogeneous solution of (2,6-dimethylphenyl)boronic acid (approximately 27.96 mg, 0.1864 mmol), N-[4-chloro-6-[4-(1-methyl-4-piperidyl)phenoxy]-5-(trifluoromethyl)pyrimidin-2-yl]-1-methyl-pyrazole-4-sulfonamide (33 mg, 0.06215 mmol), potassium carbonate (approximately 34.36 mg, 0.2486 mmol), and tetrakis(triphenylphosphine)palladium(0) (approximately 14.36 mg, 0.01243 mmol) in dioxane (259.0 μL) and water (51.79 μL) was microwaved to 120° C. for 20 minutes. The reaction mixture was diluted with water (0.10 mL), acidified with acetic acid (approximately 37.2532 mg, 35.34 μL, 0.6215 mmol), and filtered through a 0.45 μM PTFE syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-(2,6-dimethylphenyl)-6-[4-(1-methyl-4-piperidyl)phenoxy]-5-(trifluoromethyl)pyrimidin-2-yl]-1 -methyl-pyrazole-4-sulfonamide (hydrochloride salt) as a white solid (3.4 mg, 9%). ESI-MS m z calc. 600.2131, found 601.37 (M+1)⁺; Retention time: 1.42 minutes; LC method A.

### Example 187: Characterization of Compounds 498-528

The compounds in the following tables were prepared in a manner analogous to that described above using commercially available reagents and intermediates described herein.

| Compound | | LCMS Rt | Calc. | | LCMS |
|---|---|---|---|---|---|
| number | Structure | (min) | mass | M + 1 | Method |
| 498 | 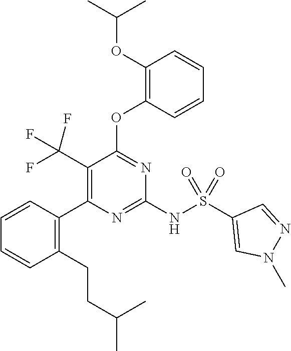 [SMILES: CC(C)CCc1ccccc1-c1nc(NS(=O)(=O)c2cnn(C)c2)nc(Oc2ccccc2OC(C)C)c1C(F)(F)F] | 2.34 | 603.213 | 604.3 | A |
| 499 | 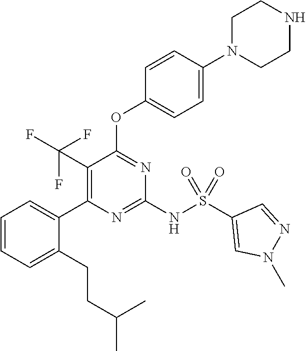 [SMILES: CC(C)CCc1ccccc1-c1nc(NS(=O)(=O)c2cnn(C)c2)nc(Oc2ccc(N3CCNCC3)cc2)c1C(F)(F)F] | 1.75 | 629.24 | 630.34 | A |
| 500 | 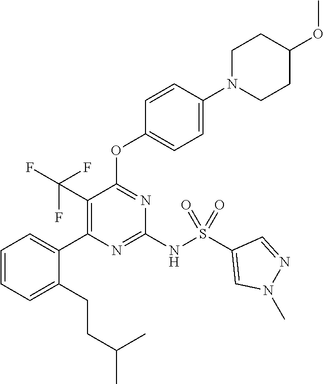 [SMILES: COC1CCN(c2ccc(Oc3nc(NS(=O)(=O)c4cnn(C)c4)nc(-c4ccccc4CCC(C)C)c3C(F)(F)F)cc2)CC1] | 2.02 | 658.255 | 659.35 | A |
| 501 | 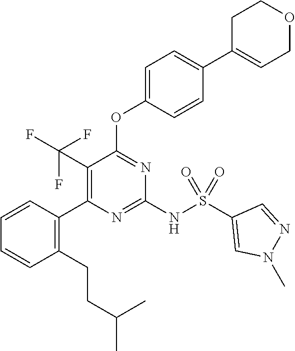 [SMILES: CC(C)CCc1ccccc1-c1nc(NS(=O)(=O)c2cnn(C)c2)nc(Oc2ccc(C3=CCOCC3)cc2)c1C(F)(F)F] | 2.28 | 627.213 | 628.29 | A |
| 502 | 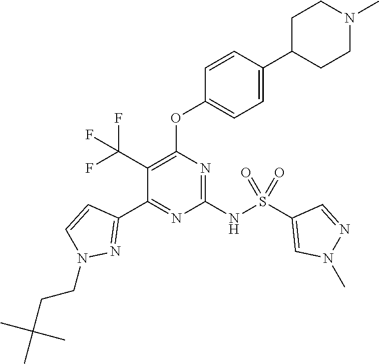 [SMILES: CN1CCC(c2ccc(Oc3nc(NS(=O)(=O)c4cnn(C)c4)nc(-c4ccn(CCC(C)(C)C)n4)c3C(F)(F)F)cc2)CC1] | 1.63 | 646.266 | 647 | A |
| 503 | 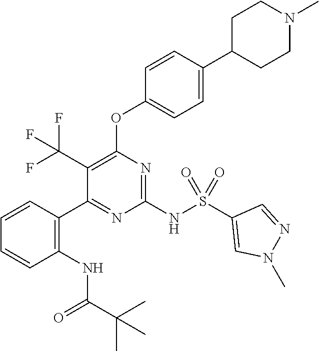 [SMILES: CN1CCC(c2ccc(Oc3nc(NS(=O)(=O)c4cnn(C)c4)nc(-c4ccccc4NC(=O)C(C)(C)C)c3C(F)(F)F)cc2)CC1] | 1.48 | 671.25 | 672 | A |
| 504 | 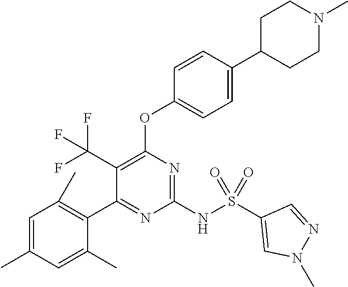 | 1.55 | 614.229 | 615 | A |
| 505 | 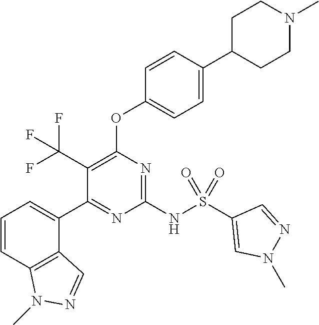 [SMILES: CN1CCC(c2ccc(Oc3nc(NS(=O)(=O)c4cnn(C)c4)nc(-c4cccc5c4cnn5C)c3C(F)(F)F)cc2)CC1] | 1.4 | 626.204 | 627 | A |
| 506 | 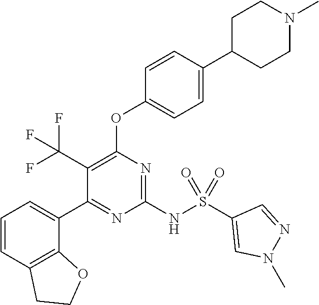 [SMILES: CN1CCC(c2ccc(Oc3nc(NS(=O)(=O)c4cnn(C)c4)nc(-c4cccc5c4OCC5)c3C(F)(F)F)cc2)CC1] | 1.46 | 614.192 | 615 | A |
| 507 | 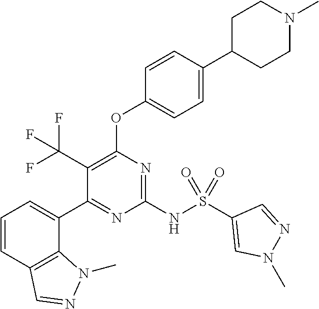 [SMILES: CN1CCC(c2ccc(Oc3nc(NS(=O)(=O)c4cnn(C)c4)nc(-c4cccc5cnn(C)c45)c3C(F)(F)F)cc2)CC1] | 1.42 | 626.204 | 627 | A |
| 508 | 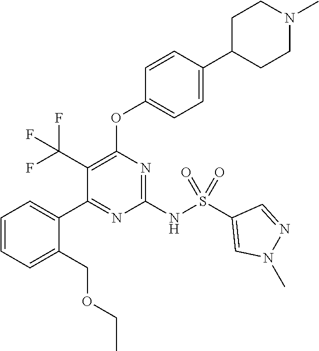 [SMILES: CCOCc1ccccc1-c1nc(NS(=O)(=O)c2cnn(C)c2)nc(Oc2ccc(C3CCN(C)CC3)cc2)c1C(F)(F)F] | 1.54 | 630.224 | 631 | A |
| 509 | 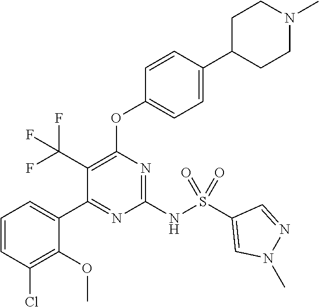 [SMILES: COc1c(Cl)cccc1-c1nc(NS(=O)(=O)c2cnn(C)c2)nc(Oc2ccc(C3CCN(C)CC3)cc2)c1C(F)(F)F] | 1.6 | 636.153 | 637 | A |
| 510 | 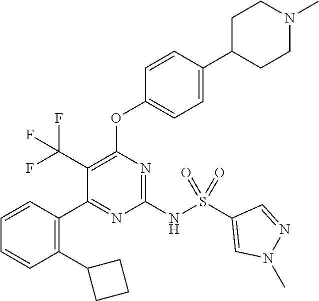 [SMILES: CN1CCC(c2ccc(Oc3nc(NS(=O)(=O)c4cnn(C)c4)nc(-c4ccccc4C4CCC4)c3C(F)(F)F)cc2)CC1] | 1.73 | 626.229 | 627 | A |
| 511 | 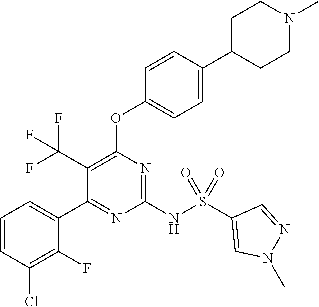 [SMILES: CN1CCC(c2ccc(Oc3nc(NS(=O)(=O)c4cnn(C)c4)nc(-c4cccc(Cl)c4F)c3C(F)(F)F)cc2)CC1] | 1.62 | 624.133 | 625 | A |
| 512 | 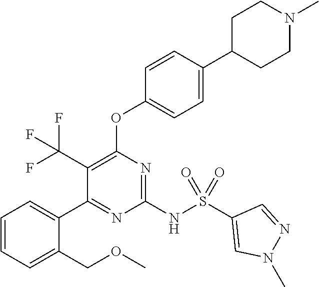 [SMILES: COCc1ccccc1-c1nc(NS(=O)(=O)c2cnn(C)c2)nc(Oc2ccc(C3CCN(C)CC3)cc2)c1C(F)(F)F] | 1.47 | 616.208 | 617 | A |
| 513 | 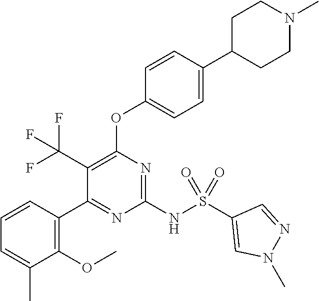 [SMILES: COc1c(C)cccc1-c1nc(NS(=O)(=O)c2cnn(C)c2)nc(Oc2ccc(C3CCN(C)CC3)cc2)c1C(F)(F)F] | 1.53 | 616.208 | 617 | A |
| 514 | 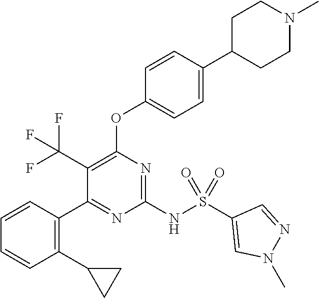 [SMILES: CN1CCC(c2ccc(Oc3nc(NS(=O)(=O)c4cnn(C)c4)nc(-c4ccccc4C4CC4)c3C(F)(F)F)cc2)CC1] | 1.59 | 612.213 | 613 | A |
| 515 | 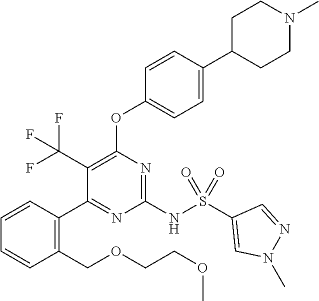 [SMILES: COCCOCc1ccccc1-c1nc(NS(=O)(=O)c2cnn(C)c2)nc(Oc2ccc(C3CCN(C)CC3)cc2)c1C(F)(F)F] | 1.58 | 660.234 | 661 | A |
| 516 | 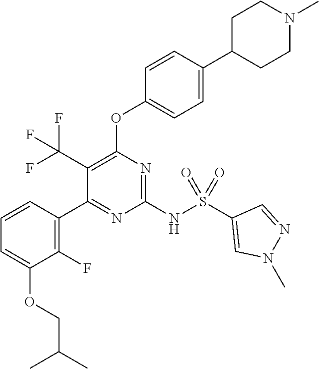 [SMILES: CC(C)COc1cccc(-c2nc(NS(=O)(=O)c3cnn(C)c3)nc(Oc3ccc(C4CCN(C)CC4)cc3)c2C(F)(F)F)c1F] | 1.78 | 662.23 | 663 | A |
| 517 | 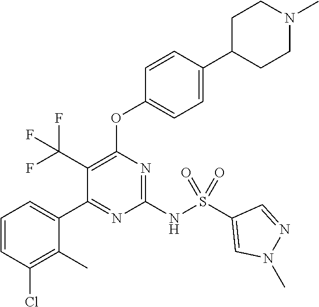 [SMILES: Cc1c(Cl)cccc1-c1nc(NS(=O)(=O)c2cnn(C)c2)nc(Oc2ccc(C3CCN(C)CC3)cc2)c1C(F)(F)F] | 1.71 | 620.158 | 621 | A |
| 518 | 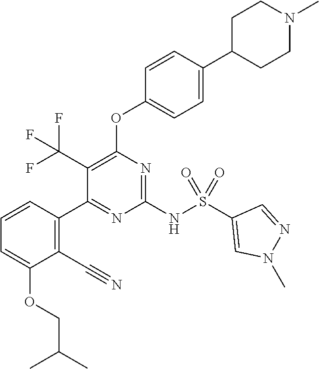 [SMILES: CC(C)COc1cccc(-c2nc(NS(=O)(=O)c3cnn(C)c3)nc(Oc3ccc(C4CCN(C)CC4)cc3)c2C(F)(F)F)c1C#N] | 1.69 | 669.235 | 670 | A |
| 519 | 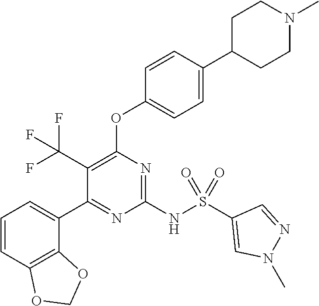 [SMILES: CN1CCC(c2ccc(Oc3nc(NS(=O)(=O)c4cnn(C)c4)nc(-c4cccc5c4OCO5)c3C(F)(F)F)cc2)CC1] | 1.49 | 616.172 | 617 | A |
| 520 | 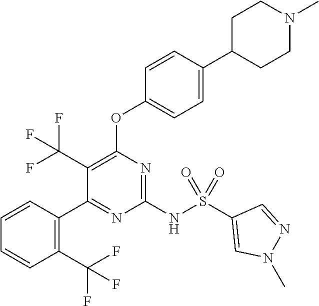 [SMILES: CN1CCC(c2ccc(Oc3nc(NS(=O)(=O)c4cnn(C)c4)nc(-c4ccccc4C(F)(F)F)c3C(F)(F)F)cc2)CC1] | 1.55 | 640.169 | 641 | A |
| 521 | 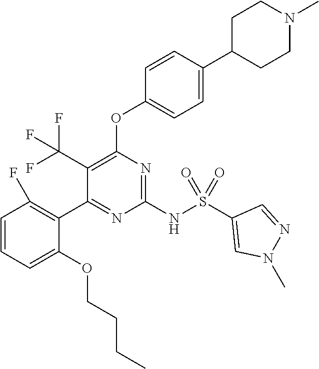 [SMILES: CCCCOc1cccc(F)c1-c1nc(NS(=O)(=O)c2cnn(C)c2)nc(Oc2ccc(C3CCN(C)CC3)cc2)c1C(F)(F)F] | 1.7 | 662.23 | 663 | A |
| 522 | 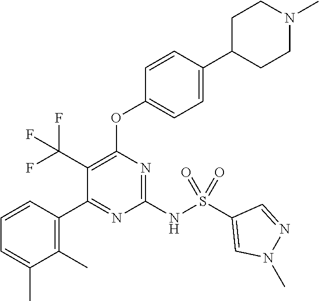 [SMILES: Cc1cccc(-c2nc(NS(=O)(=O)c3cnn(C)c3)nc(Oc3ccc(C4CCN(C)CC4)cc3)c2C(F)(F)F)c1C] | 1.6 | 600.213 | 601 | A |
| 523 | 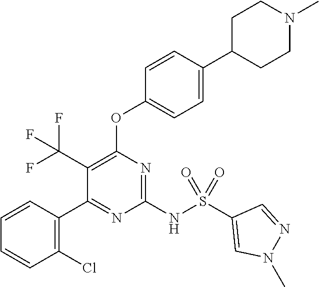 [SMILES: CN1CCC(c2ccc(Oc3nc(NS(=O)(=O)c4cnn(C)c4)nc(-c4ccccc4Cl)c3C(F)(F)F)cc2)CC1] | 1.52 | 606.143 | 607 | A |
| 524 | 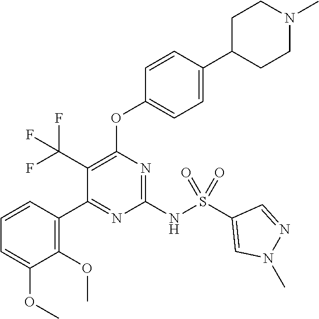 [SMILES: COc1cccc(-c2nc(NS(=O)(=O)c3cnn(C)c3)nc(Oc3ccc(C4CCN(C)CC4)cc3)c2C(F)(F)F)c1OC] | 1.5 | 632.203 | 633 | A |
| 525 | 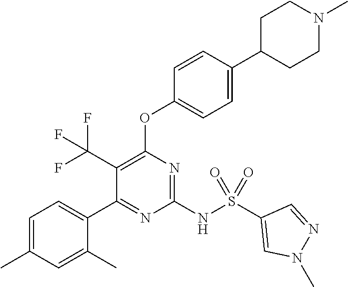 | 1.65 | 600.213 | 601 | A |
| 526 | 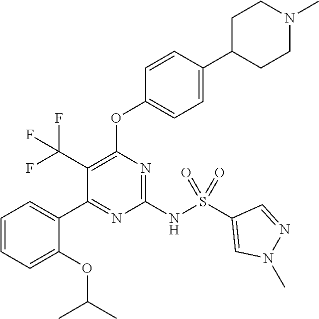 [SMILES: CC(C)Oc1ccccc1-c1nc(NS(=O)(=O)c2cnn(C)c2)nc(Oc2ccc(C3CCN(C)CC3)cc2)c1C(F)(F)F] | 1.62 | 630.224 | 631 | A |
| 527 | 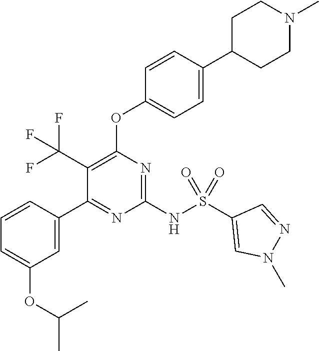 [SMILES: CC(C)Oc1cccc(-c2nc(NS(=O)(=O)c3cnn(C)c3)nc(Oc3ccc(C4CCN(C)CC4)cc3)c2C(F)(F)F)c1] | 1.68 | 630.224 | 631 | A |
| 528 | 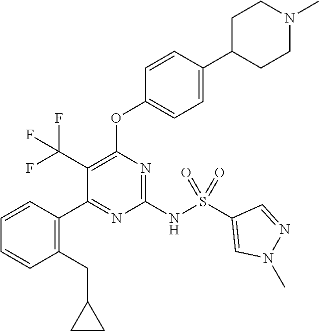 [SMILES: CN1CCC(c2ccc(Oc3nc(NS(=O)(=O)c4cnn(C)c4)nc(-c4ccccc4CC4CC4)c3C(F)(F)F)cc2)CC1] | 1.7 | 626.229 | 627 | A |

| Compound | |
|---|---|
| number | NMR |
| 503 | ¹H NMR (400 MHz, DMSO-d₆) δ 12.21 (s, 1H), 10.26 (s, 1H), 9.00 (s, |
| | 1H), 7.56 (d, J = 8.1 Hz, 1H), 7.44 (d, J = 6.7 Hz, 4H), 7.28 (d, J = |
| | 26.2 Hz, 4H), 6.93 (s, 1H), 3.72 (s, 3H), 3.51 (d, J = 11.9 Hz, 2H), 3.10 |
| | (s, 2H), 2.92 (s, 1H), 2.79 (s, 3H), 2.02 (s, 4H), 1.08 (s, 9H). |
| 505 | ¹H NMR (400 MHz, DMSO-d₆) δ 12.24 (s, 1H), 10.11 (s, 1H), 8.01 (s, |
| | 1H), 7.81 (d, J = 8.5 Hz, 1H), 7.55-7.49 (m, 2H), 7.45 (d, J = 8.6 Hz, |
| | 2H), 7.36 (d, J = 8.6 Hz, 2H), 7.17 (d, J = 7.0 Hz, 1H), 6.99 (s, 1H), |
| | 4.12 (s, 3H), 3.72 (s, 3H), 3.52 (d, J = 11.7 Hz, 2H), 3.09 (d, J = 12.3 |
| | Hz, 2H), 2.93 (s, 1H), 2.80 (d, J = 4.5 Hz, 3H), 2.04 (s, 4H). |
| 506 | ¹H NMR (400 MHz, DMSO-d₆) δ 12.21 (s, 1H), 10.27 (s, 1H), 7.52 (s, |
| | 1H), 7.43 (d, J = 8.5 Hz, 2H), 7.37 (d, J = 7.2 Hz, 1H), 7.31 (d, J = 8.5 |
| | Hz, 2H), 7.12 (d, J = 7.5 Hz, 1H), 7.02 (s, 1H), 6.95 (t, J = 7.5 Hz, 1H), |
| | 4.53 (s, 2H), 3.75 (s, 3H), 3.51 (d, J = 11.2 Hz, 3H), 3.25 (s, 2H), 3.09 |
| | (s, 2H), 2.90 (s, 2H), 2.79 (s, 4H), 2.04 (s, 4H). |
| 507 | ¹H NMR (400 MHz, DMSO-d₆) δ 10.00 (s, 1H), 8.21 (s, 1H), 7.94 (d, |
| | J = 7.4 Hz, 1H), 7.42 (s, 2H), 7.38-7.22 (m, 5H), 7.06 (s, 1H), 3.72 (s, |
| | 3H), 3.65 (s, 2H), 3.54 (d, J = 11.7 Hz, 2H), 3.11 (s, 2H), 2.93 (s, 1H), |
| | 2.83 (s, 3H), 2.03 (d, J = 24.2 Hz, 4H), 1.28 (s, 2H). |
| 509 | ¹H NMR (400 MHz, DMSO-d₆) δ 12.31 (s, 1H), 10.19 (s, 1H), 7.64 (d, |
| | J = 9.2 Hz, 1H), 7.52 (s, 1H), 7.45 (d, J = 8.4 Hz, 2H), 7.31 (dd, J = |
| | 22.9, 8.2 Hz, 5H), 6.98 (s, 1H), 3.75 (s, 3H), 3.66 (s, 3H), 3.51 (d, J = |
| | 12.3 Hz, 2H), 3.08 (s, 2H), 2.93 (s, 1H), 2.79 (s, 3H), 2.04 (s, 4H). |
| 510 | ¹H NMR (400 MHz, DMSO-d₆) δ 12.24 (s, 1H), 10.27 (s, 1H), 7.99 (s, |
| | 1H), 7.53-7.48 (m, 2H), 7.47-7.42 (m, 2H), 7.38-7.32 (m, 2H), 7.32- |
| | 7.25 (m, 2H), 7.14 (d, J = 7.9 Hz, 1H), 6.97 (s, 1H), 3.84 (s, 1H), 3.74 |
| | (s, 3H), 3.51 (d, J = 12.1 Hz, 2H), 3.44 (s, 1H), 3.08 (s, 2H), 2.91 (s, |
| | 1H), 2.79 (s, 3H), 2.27 (s, 1H), 2.11 (s, 1H), 2.01 (d, J = 35.7 Hz, 8H), |
| | 1.74 (s, 1H). |
| 511 | ¹H NMR (400 MHz, DMSO-d₆) δ 12.35 (s, 1H), 10.19 (s, 1H), 7.79 (s, |
| | 1H), 7.52 (s, 1H), 7.45 (d, J = 8.6 Hz, 3H), 7.39 (d, J = 7.9 Hz, 1H), |
| | 7.34 (d, J = 8.5 Hz, 2H), 6.98 (s, 1H), 3.76 (s, 3H), 3.51 (d, J = 11.9 |
| | Hz, 2H), 3.09 (s, 2H), 2.91 (s, 1H), 2.79 (s, 3H), 2.03 (s, 3H). |
| 512 | ¹H NMR (400 MHz, DMSO-d₆) δ 12.21 (s, 1H), 10.46 (s, 1H), 7.51 (d, |
| | J = 7.2 Hz, 3H), 7.43 (t, J = 8.1 Hz, 3H), 7.32 (d, J = 8.5 Hz, 2H), 7.26 |
| | (d, J = 7.4 Hz, 1H), 6.99 (s, 1H), 4.42 (d, J = 12.6 Hz, 1H), 4.29 (d, J = |
| | 12.7 Hz, 1H), 3.73 (s, 3H), 3.50 (d, J = 11.8 Hz, 2H), 3.12 (s, 5H), |
| | 2.92 (s, 1H), 2.78 (s, 3H), 2.04 (s, 4H). |
| 513 | ¹H NMR (400 MHz, DMSO-d₆) δ 12.24 (s, 1H), 10.18 (s, 1H), 7.53 (s, |
| | 1H), 7.44 (d, J = 8.6 Hz, 2H), 7.34 (t, J = 8.3 Hz, 3H), 7.16-7.09 (m, |
| | 2H), 7.00 (s, 1H), 3.75 (s, 3H), 3.52 (s, 5H), 3.08 (s, 2H), 2.92 (s,1H), |
| | 2.79 (s, 3H), 2.31 (s, 3H), 2.04 (s, 4H). |
| 514 | ¹H NMR (400 MHz, DMSO-d₆) δ 12.27 (s, 1H), 10.24 (s, 1H), 7.42 (s, |
| | 3H), 7.39-7.30 (m, 3H), 7.24 (t, J = 7.4 Hz, 1H), 7.16 (d, J = 7.4 Hz, |
| | 1H), 6.98 (d, J = 7.4 Hz, 2H), 3.73 (s, 3H), 3.50 (d, J = 11.5 Hz, 2H), |
| | 3.08 (s, 2H), 2.90 (s, 1H), 2.79 (s, 3H), 2.02 (s, 4H), 1.64 (s, 1H), 0.84 |
| | (d, J = 36.5 Hz, 4H), 0.54 (s, 1H). |
| 515 | ¹H NMR (400 MHz, DMSO-d₆) δ 12.19 (s, 1H), 10.43 (s, 1H), 7.55- |
| | 7.37 (m, 6H), 7.31 (d, J = 8.4 Hz, 2H), 7.25 (d, J = 7.4 Hz, 1H), 6.99 |
| | (s, 1H), 4.51 (d, J = 12.3 Hz, 1H), 4.39 (d, J = 12.3 Hz, 1H), 3.72 (s, |
| | 3H), 3.50 (d, J = 11.6 Hz, 2H), 3.39 (d, J = 6.3 Hz, 1H), 3.27 (s, 3H), |
| | 3.12 (s, 5H), 2.91 (s, 1H), 2.78 (s, 3H), 2.03 (s, 4H). |
| 517 | ¹H NMR (400 MHz, DMSO-d₆) δ 12.30 (s, 1H), 10.37 (s, 1H), 7.56 (d, |
| | J = 8.0 Hz, 1H), 7.50 (s, 1H), 7.44 (d, J = 8.4 Hz, 2H), 7.34 (d, J = 7.9 |
| | Hz, 3H), 7.22 (d, J = 7.5 Hz, 1H), 6.97 (s, 1H), 3.75 (s, 3H), 3.50 (d, |
| | J = 11.6 Hz, 3H), 3.08 (s, 2H), 2.91 (s, 2H), 2.78 (s, 4H), 2.13 (s, 3H), |
| | 2.03 (s, 4H). |
| 520 | ¹H NMR (400 MHz, DMSO-d₆) δ 12.33 (s, 1H), 9.89 (s, 1H), 7.89 (d, |
| | J = 7.7 Hz, 1H), 7.80 (t, J = 7.3 Hz, 1H), 7.73 (t, J = 7.5 Hz, 1H), 7.54 |
| | (d, J = 7.3 Hz, 1H), 7.46 (d, J = 8.5 Hz, 3H), 7.33 (d, J = 8.5 Hz, 2H), |
| | 6.89 (s, 1H), 3.74 (s, 3H), 3.52 (d, J = 11.7 Hz, 2H), 3.09 (s, 2H), 2.92 |
| | (s, 1H), 2.81 (s, 3H), 1.99 (dt, J = 35.2, 12.6 Hz, 4H). |
| 521 | ¹H NMR (400 MHz, DMSO-d₆) δ 12.31 (s, 1H), 10.34 (s, 1H), 7.47 |
| | (dd, J = 15.1, 9.0 Hz, 4H), 7.32 (d, J = 8.4 Hz, 2H), 7.15-6.89 (m, |
| | 3H), 4.01 (s, 2H), 3.76 (s, 3H), 3.52 (s, 2H), 3.10 (s, 2H), 2.93 (s, 1H), |
| | 2.78 (s, 3H), 2.05 (s, 4H), 1.54 (s, 2H), 1.28 (s, 2H), 0.78 (t, J = 7.4 Hz, |
| | 3H). |
| 522 | ¹H NMR (400 MHz, DMSO-d₆) δ 12.22 (s, 1H), 10.34 (s, 1H), 7.48 (s, |
| | 1H), 7.44 (d, J = 8.6 Hz, 2H), 7.35 (d, J = 8.6 Hz, 2H), 7.27 (d, J = 7.5 |
| | Hz, 1H), 7.17 (t, J = 7.6 Hz, 1H), 7.02 (d, J = 7.4 Hz, 1H), 6.97 (s, 1H), |
| | 3.75 (s, 3H), 3.51 (d, J = 11.5 Hz, 2H), 3.08 (s, 2H), 2.90 (s, 1H), 2.79 |
| | (s, 3H), 2.31 (s, 3H), 2.02 (d, J = 8.9 Hz, 7H). |
| 524 | ¹H NMR (400 MHz, DMSO-d₆) δ 12.25 (s, 1H), 10.09 (s, 1H), 7.51- |
| | 7.42 (m, 3H), 7.33 (d, J = 8.5 Hz, 2H), 7.22-7.12 (m, 2H), 6.96 (s, |
| | 1H), 6.83 (d, J = 7.2 Hz, 1H), 3.87 (s, 3H), 3.75 (s, 3H), 3.66 (s, 3H), |
| | 3.53 (s, 3H), 3.09 (s, 2H), 2.91 (s, 1H), 2.80 (s, 3H), 2.03 (s, 4H). |
| 525 | ¹H NMR (400 MHz, DMSO-d₆) δ 12.22 (s, 1H), 10.38 (s, 1H), 7.48 (s, |
| | 1H), 7.41 (d, J = 8.1 Hz, 2H), 7.36-7.30 (m, 2H), 7.10 (d, J = 24.9 |
| | Hz, 3H), 6.96 (s, 1H), 3.72 (s, 3H), 3.50 (s, 3H), 3.09 (s, 2H), 2.90 (s, |
| | 1H), 2.77 (s, 3H), 2.32 (s, 4H), 2.05 (d, J = 25.0 Hz, 8H). |
| 526 | ¹H NMR (400 MHz, DMSO-d₆) δ 12.22 (s, 1H), 10.21 (s, 1H), 7.53 (s, |
| | 1H), 7.44 (d, J = 8.1 Hz, 3H), 7.30 (d, J = 8.5 Hz, 2H), 7.25 (d, J = 7.4 |
| | Hz, 1H), 7.11 (d, J = 8.5 Hz, 1H), 7.02 (t, J = 7.4 Hz, 1H), 6.95 (s, 1H), |
| | 4.65 (d, J = 3.6 Hz, 1H), 3.75 (s, 3H), 3.51 (d, J = 11.4 Hz, 2H), 3.08 |
| | (s, 2H), 2.93 (s, 1H), 2.79 (s, 3H), 2.04 (s, 4H), 1.21 (d, J = 6.0 Hz, |
| | 3H), 1.15 (d, J = 6.0 Hz, 3H). |
| 528 | ¹H NMR (400 MHz, DMSO-d₆) δ 12.14 (s, 1H), 10.03 (s, 1H), 7.44- |
| | 7.39 (m, 2H), 7.34 (d, J = 7.9 Hz, 3H), 7.28-7.17 (m, 3H), 7.08 (d, J = |
| | 7.5 Hz, 1H), 6.86 (s, 1H), 3.63 (s, 3H), 3.42 (s, 2H), 3.00 (s, 2H), |
| | 2.83 (s, 1H), 2.69 (s, 3H), 2.27 (d, J = 9.4 Hz, 2H), 1.94 (s, 4H), 0.81 |
| | (s, 1H), 0.33 (d, J = 7.8 Hz, 2H), −0.00 (d, J = 15.3 Hz, 2H). |

### Example 188: Preparation of Compound 529

### Step 1: N-[4-chloro-5-isopropyl-6-(2-isopropylphenyl)pyrimidin-2-yl]-3-nitro-benzenesulfonamide

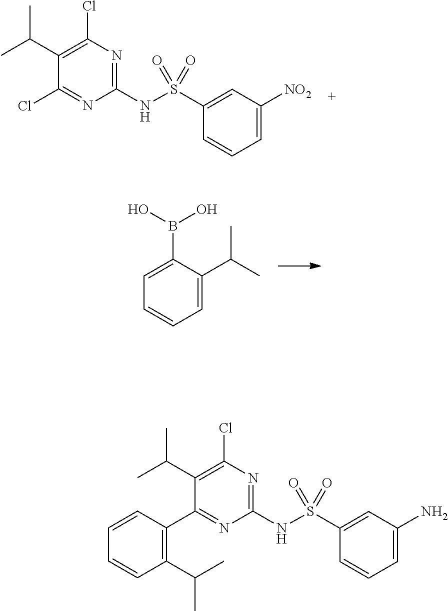 [SMILES: CC(C)c1c(Cl)nc(NS(=O)(=O)c2cccc([N+](=O)[O-])c2)nc1Cl ; CC(C)c1ccccc1B(O)O ; CC(C)c1ccccc1-c1nc(NS(=O)(=O)c2cccc(N)c2)nc(Cl)c1C(C)C]

Stage 1: A heterogeneous solution of (2-isopropylphenyl)boronic acid (148 mg, 0.9024 mmol), N-(4,6-dichloro-5-isopropyl-pyrimidin-2-yl)-3-nitro-benzenesulfonamide (352 mg, 0.8997 mmol), bis(triphenylphosphine)palladium(II) chloride (19.0 mg, 0.02707 mmol) and potassium carbonate (approximately 373.0 mg, 2.699 mmol) in dioxane (4.5 mL) was sealed in a pressure vessel and heated to 85° C. for 16 hours. The reaction mixture was acidified using acetic acid (770 μL, 13.54 mmol) and concentrated in vacuo. The crude residue was dissolved in DMSO (1.0 mL), filtered through a 0.45 μM PTFE syringe filter, and then separated by HPLC (gradient: 1 to 99% acetonitrile in water with 0.1% hydrochloric acid) to afford the intermediate N-[4-chloro-5-isopropyl-6-(2-isopropylphenyl)pyrimidin-2-yl]-3-nitro-benzenesulfonamide (60 mg, 4%) as a white solid. ESI-MS m/z calc. 474.11285, found 475.4 (M+1)⁺; Retention time: 0.84 minutes. LC method D.

Stage 2: To a solution of N-[4-chloro-5-isopropyl-6-(2-isopropylphenyl)pyrimidin-2-yl]-3-nitro-benzenesulfonamide (60 mg, 4%) in ethanol (4.5 mL) was added iron (251 mg, 4.495 mmol) and hydrochloric acid (220 μL of 37% w/v, 2.233 mmol). The reaction vessel was sealed and heated to 65° C. for 2 hours. The reaction mixture was diluted with diethyl ether, filtered through a 0.2 μM PTFE syringe filter, and concentrated in vacuo. The crude residue was dissolved in DMSO (1.0 mL) and separated by HPLC (gradient: 1 to 99% acetonitrile in water with 0.1% hydrochloric acid) to afford 3-amino-N-[4-chloro-5-isopropyl-6-(2-isopropylphenyl)pyrimidin-2-yl]benzenesulfonamide (22 mg, 5%) as a white solid. ESI-MS m z calc. 444.13867, found 445.39 (M+1)⁺; Retention time: 0.77 minutes. LC method D.

### Step 2: 3-Amino-N-[5-isopropyl-4-(2-isopropylphenyl)-6-(2-methylphenoxy)pyrimidin-2-yl]benzenesulfonamide

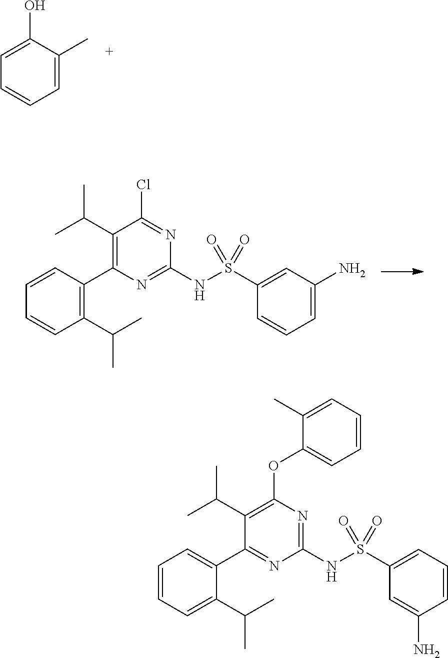

A heterogeneous solution consisting of o-cresol (approximately 8.110 mg, 14.67 μL, 0.07500 mmol), 3-amino-N-[4-chloro-5-isopropyl-6-(2-isopropylphenyl)pyrimidin-2-yl]benzenesulfonamide (11.12 mg, 0.0250 mmol), and potassium carbonate (approximately 13.82 mg, 0.1000 mmol) in NMP (125.0 μL) was heated in a sealed vial to 110° C. for 16 hours. The solution was acidified using acetic acid (approximately 45.04 mg, 42.65 μL, 0.7500 mmol), diluted with DMSO (0.5 mL), and filtered using a 0.2 μM PTFE syringe filter. The crude solution was separated by preparative HPLC (gradient: 10 to 99% acetonitrile in water with 0.1% hydrochloric acid) which furnished the title compound as a white solid. 3-amino-N-[5-isopropyl-4-(2-isopropylphenyl)-6-(2-methylphenoxy)pyrimidin-2-yl]benzenesulfonamide (3.1 mg, 24%). ESI-MS m/z calc. 516.2195, found 517.52 (M+1)⁺; Retention time: 2.13 minutes; LC method A.

### Example 189: Preparation of Compound 530

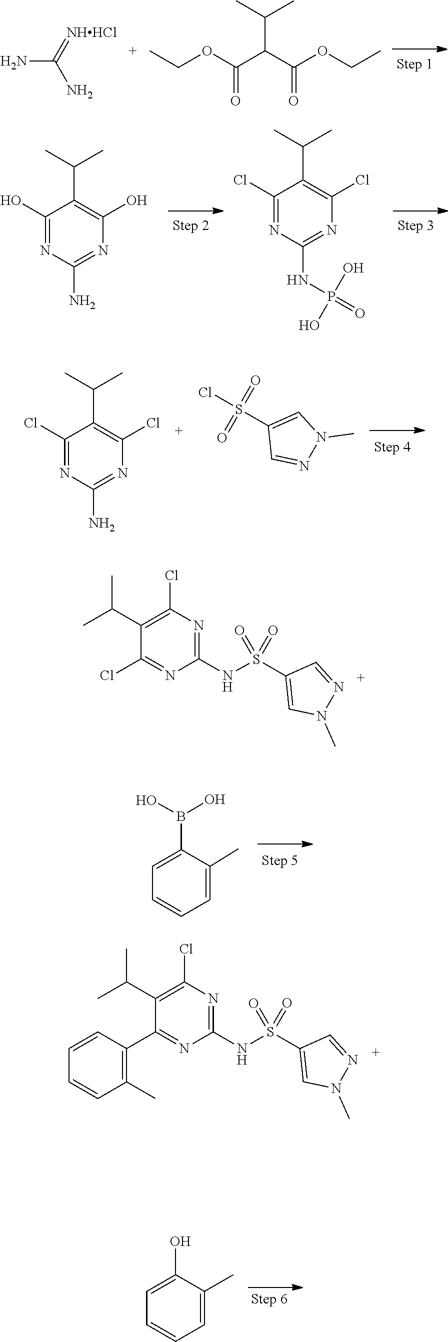

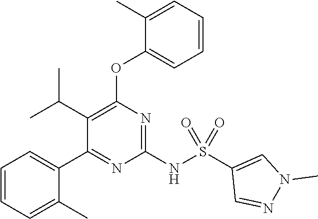 [SMILES: Cc1ccccc1Oc1nc(NS(=O)(=O)c2cnn(C)c2)nc(-c2ccccc2C)c1C(C)C]

### Step 1: 2-Amino-5-isopropyl-pyrimidine-4,6-diol

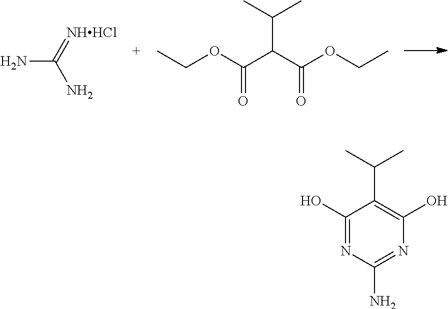

Under nitrogen atmosphere, heptane washed sodium metal (8.3 g, 361 mmol) was dissolved in absolute ethanol (380 mL) and guanidine hydrochloride (13.6 g, 142.4 mmol) was added and the resulting suspension was stirred for 5 minutes and then diethyl 2-isopropylpropanedioate (25.02 g, 25.5 mL, 123.7 mmol) was added. The reaction was then left stirring at 80° C. overnight. The reaction mixture was concentrated then dissolved in distilled water (80 mL) and the resulting solution was acidified with 3 N HCl (˜100 mL). Water was evaporated until a lot of precipitate appeared. The resulting precipitate was filtered, washed with cold water (2×20 mL) then with heptane (2×25 mL). The filtrate was evaporated, and the same treatment was done. The resulting powder was dried under reduced pressure to provide 2-amino-5-isopropyl-pyrimidine-4,6-diol hydrate (28.3 g, 122%) as a white powder. ¹H NMR (300 MHz, DMSO-d₆) δ ppm 1.09 (d, J=6.2 Hz, 6H), 2.96 (br. s., 1H), 3.31-3.72 (m, 1H), 7.28 (br. s., 2H), 8.74-10.34 (m, 4H). ESI-MS m/z calc. 169.181, found 170.2 (M+1)⁺; Retention time: 0.49 minutes (LC method C).

### Step 2: [(4,6-Dichloro-5-isopropyl-pyrimidin-2-yl)amino]phosphonic acid

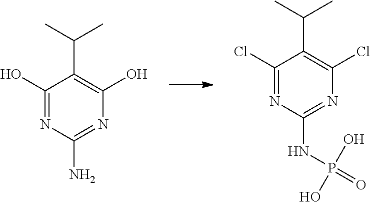 [SMILES: CC(C)c1c(O)nc(N)nc1O ; CC(C)c1c(Cl)nc(NP(=O)(O)O)nc1Cl]

Phosphoryl trichloride (123.4 g, 75 mL, 804.8 mmol) was added to a suspension of 2-amino-5-isopropyl-pyrimidine-4,6-diol hydrate (23.3 g, 124.5 mmol) in dioxane (460 mL) at room temperature. The reaction mixture was heated at 95-100° C. for 23 hours. The reaction mixture was concentrated under reduced pressure and left over night on high vacuum pump to provide the crude desired product [(4,6-dichloro-5-isopropyl-pyrimidin-2-yl)amino]phosphonic acid (58.3 g, 164%) as a brown oil that was used without further purification in the next step.

### Step 3: 4,6-Dichloro-5-isopropyl-pyrimidin-2-amine

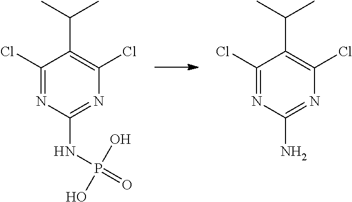 [SMILES: CC(C)c1c(Cl)nc(NP(=O)(O)O)nc1Cl ; CC(C)c1c(Cl)nc(N)nc1Cl]

[(4,6-Dichloro-5-isopropyl-pyrimidin-2-yl)amino]phosphonic acid (35.61 g, 124.5 mmol) (58.3 g) was dissolved in tetrahydrofuran (360 mL) and HCl (325 mL of 3 M, 975.0 mmol) was added and the reaction mixture was stirred at 70° C. for 7 hours. Then it was cooled to room temperature and diluted with brine (500 mL) and extracted with ethyl acetate (500 mL, ×3). The organic phases were combined, dried over anhydrous sodium sulfate, filtered and concentrated under reduced pressure. The resulting crude semi-solid mixture was purified by silica-gel column chromatography on a 330 g column, eluting from 0% to 40% of ethyl acetate in heptane then, after concentration, the residue was triturated in acetonitrile to provide 4,6-dichloro-5-isopropyl-pyrimidin-2-amine (4.8 g, 19%) as an off-white solid. ¹H NMR (300 MHz, DMSO-d₆) δ ppm 1.26 (d, J=7.0 Hz, 6H), 3.38-3.51 (m, 1H), 7.29 (s, 2H). ESI-MS m/z found 192.1 (M+1)⁺; Retention time: 2.50 minutes (LC method B).

### Step 4: N-(4,6-Dichloro-5-isopropyl-pyrimidin-2-yl)-1-methyl-pyrazole-4-sulfonamide

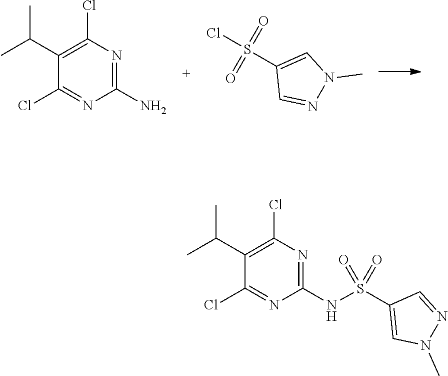

To a solution of 4,6-dichloro-5-isopropyl-pyrimidin-2-amine (1.0 g, 4.853 mmol) in DMF (20 mL) at 0° C. was added sodium hydride (approximately 776.3 mg of 60% w/w, 19.41 mmol). After stirring for 15 minutes, 1-methylpyrazole-4-sulfonyl chloride (approximately 1.753 g, 9.706 mmol) was added in one portion. The solution was allowed to warm to 23° C. over 15 minutes and then cooled back to 0° C. The reaction was acidified using acetic acid (approximately 4.372 g, 4.140 mL, 72.80 mmol) and partitioned in ethyl acetate and water. The organic layer was removed, and the aqueous layer was further extracted with ethyl acetate (5×). The combined organics were washed twice with brine, dried over magnesium sulfate, filtered, and concentrated in vacuo. The crude residue was subjected to flash column chromatography on silica gel (ethyl acetate in hexanes) to afford N-(4,6-dichloro-5-isopropyl-pyrimidin-2-yl)-1 -methyl-pyrazole-4-sulfonamide (2.28 g, 86%) as a white foam. ESI-MS m/z calc. 349.0167, found 350.32 (M+1)⁺; Retention time: 0.61 minutes; LC method D.

### Step 5: N-[4-chloro-5-isopropyl-6-(o-tolyl)pyrimidin-2-yl]-1-methyl-pyrazole-4-sulfonamide

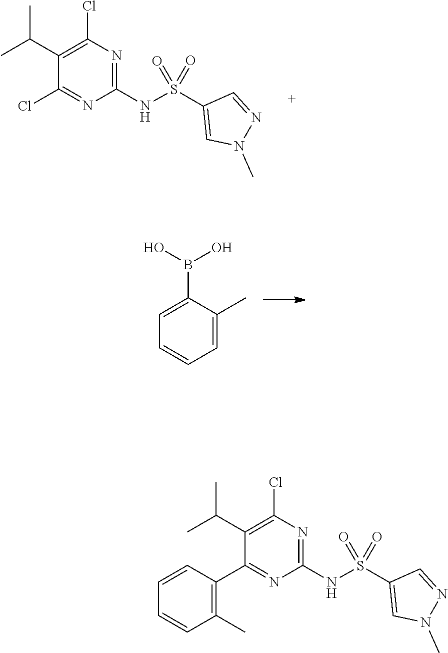

A heterogeneous solution of o-tolylboronic acid (122.4 mg, 0.9003 mmol), N-(4,6-dichloro-5-isopropyl-pyrimidin-2-yl)-1-methyl-pyrazole-4-sulfonamide (492.5 mg, 0.9000 mmol), bis(triphenylphosphine)palladium(II) dichloride (19.0 mg, 0.02707 mmol), and potassium carbonate (373.2 mg, 2.700 mmol) in dioxane (2.1 mL) was sealed in a pressure vessel and heated to 85° C. for 16 hours. The reaction mixture was acidified using acetic acid (810.7 mg, 13.50 mmol) and then concentrated in vacuo onto silica gel. The crude impregnated silica gel was separated by flash column chromatography (ethyl acetate in hexanes) to afford N-[4-chloro-5-isopropyl-6-(o-tolyl)pyrimidin-2-yl]-1-methyl-pyrazole-4-sulfonamide (200 mg, 32%) as a white solid. ESI-MS m/z calc. 405.10263, found 406.34 (M+1)⁺; Retention time: 0.72 minutes; LC method D.

### Step 6: N-[5-isopropyl-4-(2-methylphenoxy)-6-(o-tolyl)pyrimidin-2-yl]-1-methyl-pyrazole-4-sulfonamide

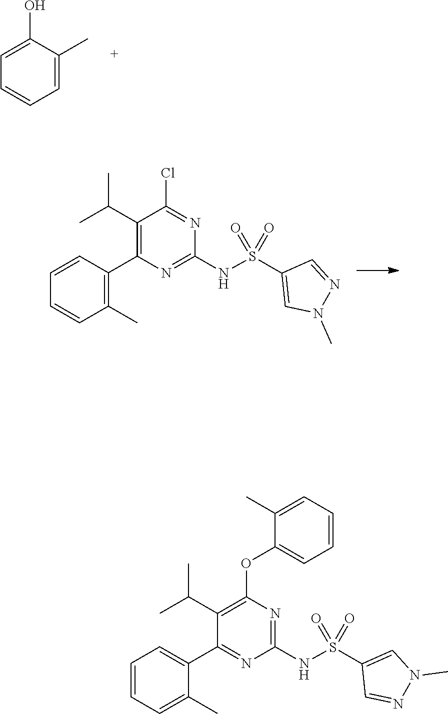

A heterogeneous solution consisting of o-cresol (approximately 8.110 mg, 14.67 μL, 0.07500 mmol), N-[4-chloro-5-isopropyl-6-(o-tolyl)pyrimidin-2-yl]-1-methyl-pyrazole-4-sulfonamide (10.15 mg, 0.0250 mmol), and potassium carbonate (approximately 13.82 mg, 0.1000 mmol) in NMP (125.0 μL) was heated in a sealed vial to 110° C. for 16 hours. The solution was acidified using acetic acid (approximately 45.04 mg, 42.65 μL, 0.7500 mmol), diluted with DMSO (0.5 mL), and filtered using a 0.2 μM PTFE syringe filter. The crude solution was separated by prep HPLC (gradient: 10 to 99% acetonitrile in water with 0.1% hydrochloric acid) which furnished the title compound as a white solid. N-[5-isopropyl-4-(2-methylphenoxy)-6-(o-tolyl)pyrimidin-2-yl]-1-methyl-pyrazole-4-sulfonamide (10.9 mg, 91%). ESI-MS m/z calc. 477.18347, found 478.1 (M+1)⁺; Retention time: 1.92 minutes; LC method A.

### Example 190: Preparation of Compound 531

### Step 1: 3-Amino-N-[5-isopropyl-4-(2-methylphenoxy)-6-(o-tolyl)pyrimidin-2-yl]benzenesulfonamide

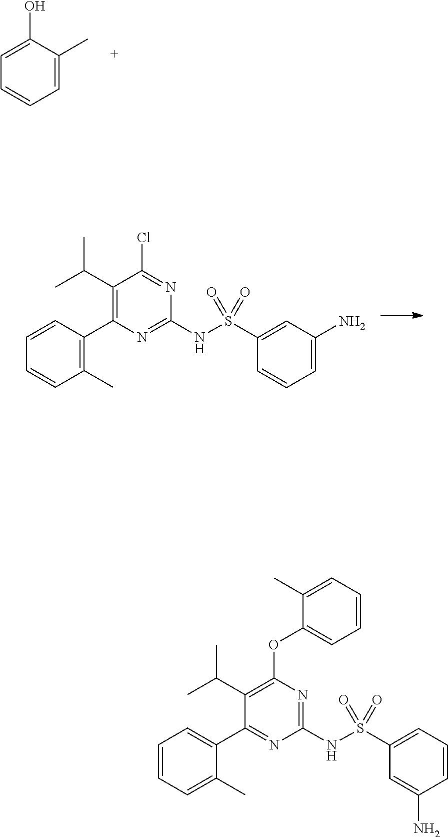

A heterogeneous solution consisting of o-cresol (approximately 8.110 mg, 14.67 μL, 0.07500 mmol), 3-amino-N-[4-chloro-5-isopropyl-6-(o-tolyl)pyrimidin-2-yl]benzenesulfonamide (10.42 mg, 0.0250 mmol), and potassium carbonate (approximately 13.82 mg, 0.1000 mmol) in NMP (125.0 μL) was heated in a sealed vial to 110° C. for 16 hours. The solution was acidified using acetic acid (approximately 45.04 mg, 42.65 μL, 0.7500 mmol), diluted with DMSO (0.5 mL), and filtered using a 0.2 μM PTFE syringe filter. The crude solution was separated by prep HPLC (gradient: 10 to 99% acetonitrile in water with 0.1% hydrochloric acid) which furnished the title compound as a white solid. 3-Amino-N-[5-isopropyl-4-(2-methylphenoxy)-6-(o-tolyl)pyrimidin-2-yl]benzenesulfonamide (4.3 mg, 35%). ESI-MS m/z calc. 488.1882, found 489.51 (M+1)⁺; Retention time: 1.93 minutes; LC method A.

### Example 191: Preparation of Compound 532

### Step 1: 3-Amino-N-[5-isopropyl-4-(2-isopropylphenyl)-6-phenoxy-pyrimidin-2-yl]benzenesulfonamide

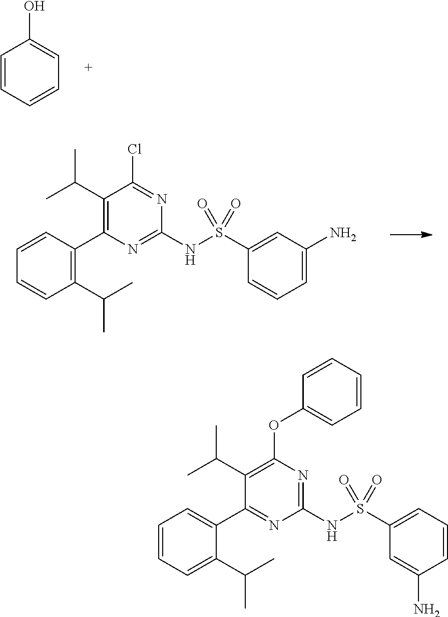 [SMILES: Oc1ccccc1 ; CC(C)c1ccccc1-c1nc(NS(=O)(=O)c2cccc(N)c2)nc(Cl)c1C(C)C ; CC(C)c1ccccc1-c1nc(NS(=O)(=O)c2cccc(N)c2)nc(Oc2ccccc2)c1C(C)C]

A heterogeneous solution consisting of phenol (approximately 7.058 mg, 6.658 μL, 0.07500 mmol), 3-amino-N-[4-chloro-5-isopropyl-6-(2-isopropylphenyl)pyrimidin-2-yl]benzenesulfonamide (11.12 mg, 0.0250 mmol), and potassium carbonate (approximately 13.82 mg, 0.1000 mmol) in NMP (125.0 μL) was heated in a sealed vial to 110° C. for 16 hours. The solution was acidified using acetic acid (approximately 45.04 mg, 42.65 μL, 0.7500 mmol), diluted with DMSO (0.5 mL), and filtered using a 0.2 μM PTFE syringe filter. The crude solution was separated by prep HPLC (gradient: 10 to 99% acetonitrile in water with 0.1% hydrochloric acid) which furnished the title compound as a white solid. 3-amino-N-[5-isopropyl-4-(2-isopropylphenyl)-6-phenoxy-pyrimidin-2-yl]benzenesulfonamide (3.4 mg, 27%), ESI-MS m/z calc. 502.20386, found 503.55 (M+1)⁺; Retention time: 2.05 minutes; LC method A. Example 192 Preparation of Compound 533

### Step 1: N-[4-(2-fluorophenoxy)-5-isopropyl-6-(o-tolyl)pyrimidin-2-yl]-1-methyl-pyrazole-4-sulfonamide

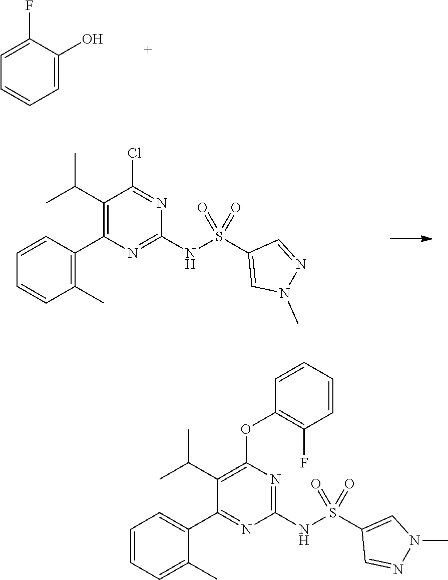

A heterogeneous solution consisting of 2-fluorophenol (approximately 8.408 mg, 0.07500 mmol), N-[4-chloro-5-isopropyl-6-(o-tolyl)pyrimidin-2-yl]-1-methyl-pyrazole-4-sulfonamide (10.15 mg, 0.0250 mmol), and potassium carbonate (approximately 13.82 mg, 0.1000 mmol) in NMP (125.0 μL) was heated in a sealed vial to 110° C. for 16 hours. The solution was acidified using acetic acid (approximately 45.04 mg, 42.65 μL, 0.7500 mmol), diluted with DMSO (0.5 mL), and filtered using a 0.2 μM PTFE syringe filter. The crude solution was separated by prep HPLC (gradient: 10 to 99% acetonitrile in water with 0.1% hydrochloric acid) which furnished the title compound as a white solid. N-[4-(2-fluorophenoxy)-5-isopropyl-6-(o-tolyl)pyrimidin-2-yl]-1-methyl-pyrazole-4-sulfonamide (6.5 mg, 54%). ESI-MS m/z calc. 481.1584, found 482.47 (M+1)⁺; Retention time: 1.89 minutes; LC method A.

### Example 193: Preparation of Compound 534

### Step 1: N-[4-[3-chloro-4-(4-methylpiperazin-1-yl)phenoxy]-5-isopropyl-6-(o-tolyl)pyrimidin-2-yl]-1-methyl-pyrazole-4-sulfonamide

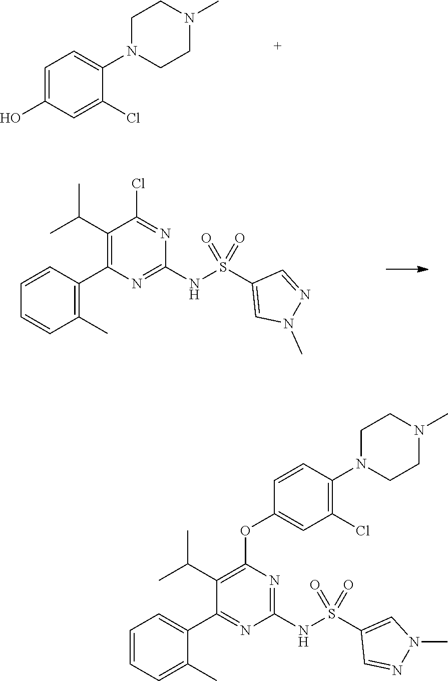 [SMILES: CN1CCN(c2ccc(O)cc2Cl)CC1 ; Cc1ccccc1-c1nc(NS(=O)(=O)c2cnn(C)c2)nc(Cl)c1C(C)C ; Cc1ccccc1-c1nc(NS(=O)(=O)c2cnn(C)c2)nc(Oc2ccc(N3CCN(C)CC3)c(Cl)c2)c1C(C)C]

A heterogeneous solution consisting of 3-chloro-4-(4-methylpiperazin-1-yl)phenol (approximately 17.00 mg, 0.07500 mmol), N-[4-chloro-5-isopropyl-6-(o-tolyl)pyrimidin-2-yl]-1-methyl-pyrazole-4-sulfonamide (10.15 mg, 0.0250 mmol), and potassium carbonate (approximately 13.82 mg, 0.1000 mmol) in NMP (125.0 μL) was heated in a sealed vial to 110° C. for 16 hours. The solution was acidified using acetic acid (approximately 45.04 mg, 42.65 μL, 0.7500 mmol), diluted with DMSO (0.5 mL), and filtered using a 0.2 μM PTFE syringe filter. The crude solution was separated by prep HPLC (gradient: 10 to 99% acetonitrile in water with 0.1% hydrochloric acid) which furnished the title compound as a white solid. N-[4-[3-chloro-4-(4-methylpiperazin-1-yl)phenoxy]-5-isopropyl-6-(o-tolyl)pyrimidin-2-yl]-1-methyl-pyrazole-4-sulfonamide (14.2 mg, 83%). ESI-MS m/z calc. 595.21326, found 596.58 (M+1)⁺; Retention time: 1.41 minutes; LC method A.

### Example 194: Preparation of Compound 535

### Step 1: N-[5-isopropyl-4-[4-(4-methylpiperazin-1-yl)phenoxy]-6-(o-tolyl)pyrimidin-2-yl]-1-methyl-pyrazole-4-sulfonamide

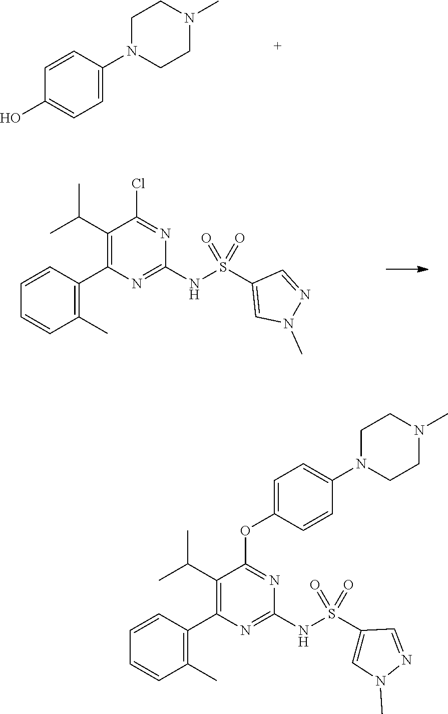 [SMILES: CN1CCN(c2ccc(O)cc2)CC1 ; Cc1ccccc1-c1nc(NS(=O)(=O)c2cnn(C)c2)nc(Cl)c1C(C)C ; Cc1ccccc1-c1nc(NS(=O)(=O)c2cnn(C)c2)nc(Oc2ccc(N3CCN(C)CC3)cc2)c1C(C)C]

A heterogeneous solution consisting of 4-(4-methylpiperazin-1-yl)phenol (approximately 14.42 mg, 0.07500 mmol), N-[4-chloro-5-isopropyl-6-(o-tolyl)pyrimidin-2-yl]-1-methyl-pyrazole-4-sulfonamide (10.15 mg, 0.0250 mmol), and potassium carbonate (approximately 13.82 mg, 0.1000 mmol) in NMP (125.0 μL) was heated in a sealed vial to 110° C. for 16 hours. The solution was acidified using acetic acid (approximately 45.04 mg, 42.65 μL, 0.7500 mmol), diluted with DMSO (0.5 mL), and filtered using a 0.2 μM PTFE syringe filter. The crude solution was separated by prep HPLC (gradient: 10 to 99% acetonitrile in water with 0.1% hydrochloric acid) which furnished the title compound as a white solid. N-[5-isopropyl-4-[4-(4-methylpiperazin-1-yl)phenoxy]-6-(o-tolyl)pyrimidin-2-yl]-1-methyl-pyrazole-4-sulfonamide (7.5 mg, 50%). ESI-MS m/z calc. 561.2522, found 562.62 (M+1)⁺; Retention time: 1.31 minutes; LC method A.

### Example 195: Preparation of Compound 536

### Step 1: N-[4-chloro-5-isopropyl-6-(2-isopropylphenyl)pyrimidin-2-yl]-1-methyl-pyrazole-4-sulfonamide

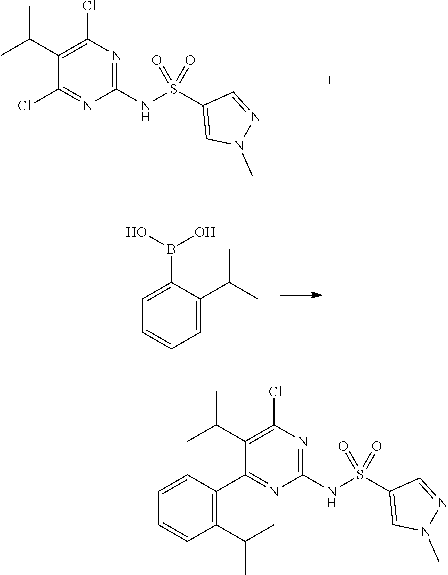 [SMILES: CC(C)c1c(Cl)nc(NS(=O)(=O)c2cnn(C)c2)nc1Cl ; CC(C)c1ccccc1B(O)O ; CC(C)c1ccccc1-c1nc(NS(=O)(=O)c2cnn(C)c2)nc(Cl)c1C(C)C]

A heterogeneous solution of (2-isopropylphenyl)boronic acid (148 mg, 0.9024 mmol), N-(4,6-dichloro-5-isopropyl-pyrimidin-2-yl)-1-methyl-pyrazole-4-sulfonamide (493 mg, 0.9009 mmol), bis(triphenylphosphine)palladium(II) dichloride (19 mg, 0.02707 mmol), and potassium carbonate (373 mg, 2.699 mmol) in dioxane (2.4 mL) was sealed in a pressure vessel and heated to 80° C. for 16 hours. The reaction mixture was diluted with dichloromethane and a saturated solution of aqueous ammonium chloride was added. The organic layer was removed, and the aqueous layer further extracted with dichloromethane (3×). The combined organics were dried over magnesium sulfate, filtered, and concentrated. The crude mixture was separated by flash column chromatography on silica gel (25% ethyl acetate in hexanes) to afford N-[4-chloro-5-isopropyl-6-(2-isopropylphenyl)pyrimidin-2-yl]-1-methyl-pyrazole-4-sulfonamide (205.0 mg, 52%) as a white solid. ESI-MS m/z calc. 433.1339, found 434.39 (M+1)⁺; Retention time: 0.77 minutes; LC method D.

### Step 2: N-[4-(2-fluorophenoxy)-5-isopropyl-6-(2-isopropylphenyl)pyrimidin-2-yl]-1 -methyl-pyrazole-4-sulfonamide

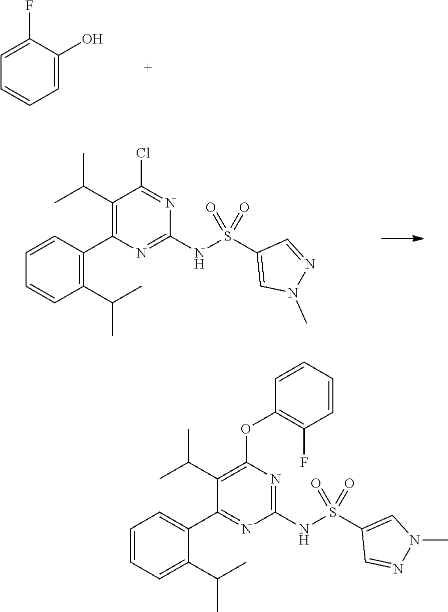

A heterogeneous solution consisting of 2-fluorophenol (approximately 8.408 mg, 0.07500 mmol), N-[4-chloro-5-isopropyl-6-(2-isopropylphenyl)pyrimidin-2-yl]-1-methyl-pyrazole-4-sulfonamide (10.85 mg, 0.0250 mmol), and potassium carbonate (approximately 13.82 mg, 0.1000 mmol) in NMP (125.0 μL) was heated in a sealed vial to 110° C. for 16 hours. The solution was acidified using acetic acid (approximately 45.04 mg, 42.65 μL, 0.7500 mmol), diluted with DMSO (0.5 mL), and filtered using a 0.2 μM PTFE syringe filter. The crude solution was separated by prep HPLC (gradient: 10 to 99% acetonitrile in water with 0.1% hydrochloric acid) which furnished the title compound as a white solid. N-[4-(2-fluorophenoxy)-5-isopropyl-6-(2-isopropylphenyl)pyrimidin-2-yl]-1-methyl-pyrazole-4-sulfonamide (6.6 mg, 52%). ESI-MS m/z calc. 509.1897, found 510.52 (M+1)⁺; Retention time: 2.1 minutes; LC method A.

### Example 196: Preparation of Compound 537

### Step 1: N-[5-isopropyl-4-(2-isopropylphenyl)-6-(2-methylphenoxy)pyrimidin-2-yl]-1-methyl-pyrazole-4-sulfonamide

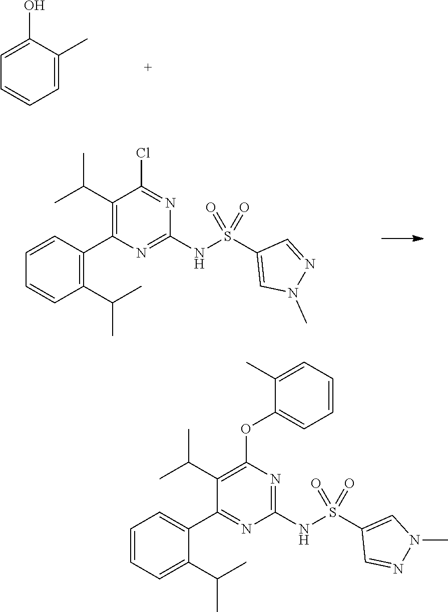

A heterogeneous solution consisting of o-cresol (approximately 8.110 mg, 14.67 μL, 0.07500 mmol), N-[4-chloro-5-isopropyl-6-(2-isopropylphenyl)pyrimidin-2-yl]-1-methyl-pyrazole-4-sulfonamide (10.85 mg, 0.0250 mmol), and potassium carbonate (approximately 13.82 mg, 0.1000 mmol) in NMP (125.0 μL) was heated in a sealed vial to 110° C. for 16 hours. The solution was acidified using acetic acid (approximately 45.04 mg, 42.65 μL, 0.7500 mmol), diluted with DMSO (0.5 mL), and filtered using a 0.2 μM PTFE syringe filter. The crude solution was separated by prep HPLC (gradient: 10 to 99% acetonitrile in water with 0.1% hydrochloric acid) which furnished the title compound as a white solid. N-[5-isopropyl-4-(2-isopropylphenyl)-6-(2-methylphenoxy)pyrimidin-2-yl]-1-methyl-pyrazole-4-sulfonamide (3.7mg, 29%). ESI-MS m/z calc. 505.21475, found 506.52 (M+1)⁺; Retention time: 2.15 minutes; LC method A.

### Example 197: Preparation of Compound 538

### Step 1: N-[4-[3-chloro-4-(4-methylpiperazin-1-yl)phenoxy]-5-isopropyl-6-(2-isopropylphenyl)pyrimidin-2-yl]-1-methyl-pyrazole-4-sulfonamide

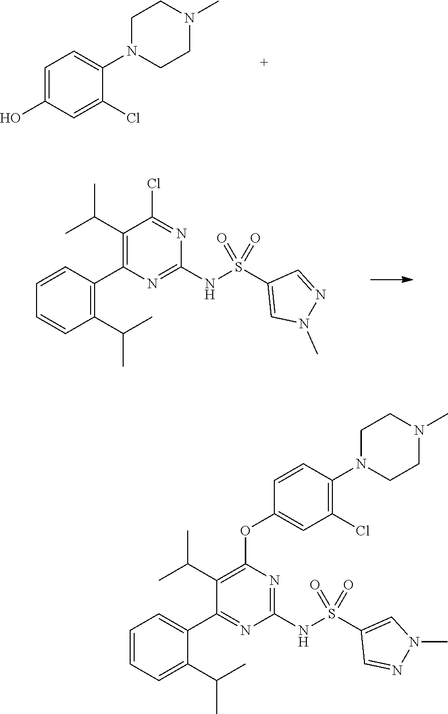 [SMILES: CN1CCN(c2ccc(O)cc2Cl)CC1 ; CC(C)c1ccccc1-c1nc(NS(=O)(=O)c2cnn(C)c2)nc(Cl)c1C(C)C ; CC(C)c1ccccc1-c1nc(NS(=O)(=O)c2cnn(C)c2)nc(Oc2ccc(N3CCN(C)CC3)c(Cl)c2)c1C(C)C]

A heterogeneous solution consisting of 3-chloro-4-(4-methylpiperazin-1-yl)phenol (approximately 17.00 mg, 0.07500 mmol), N-[4-chloro-5-isopropyl-6-(2-isopropylphenyl)pyrimidin-2-yl]-1-methyl-pyrazole-4-sulfonamide (10.85 mg, 0.0250 mmol), and potassium carbonate (approximately 13.82 mg, 0.1000 mmol) in NMP (125.0 μL) was heated in a sealed vial to 110° C. for 16 hours. The solution was acidified using acetic acid (approximately 45.04 mg, 42.65 μL, 0.7500 mmol), diluted with DMSO (0.5 mL), and filtered using a 0.2 μM PTFE syringe filter. The crude solution was separated by prep HPLC (gradient: 10 to 99% acetonitrile in water with 0.1% hydrochloric acid) which furnished the title compound as a white solid. N-[4-[3-chloro-4-(4-methylpiperazin-1-yl)phenoxy]-5-isopropyl-6-(2-isopropylphenyl)pyrimidin-2-yl]-1-methyl-pyrazole-4-sulfonamide (5.4 mg, 33%). ESI-MS m z calc. 623.2445, found 624.59 (M+1)⁺; Retention time: 1.59 minutes; LC method A.

### Example 198: Preparation of Compound 539

### Step 1: N-[5-isopropyl-4-(2-isopropylphenyl)-6-[4-(4-methylpiperazin-1-yl)phenoxy]pyrimidin-2-yl]-1-methyl-pyrazole-4-sulfonamide

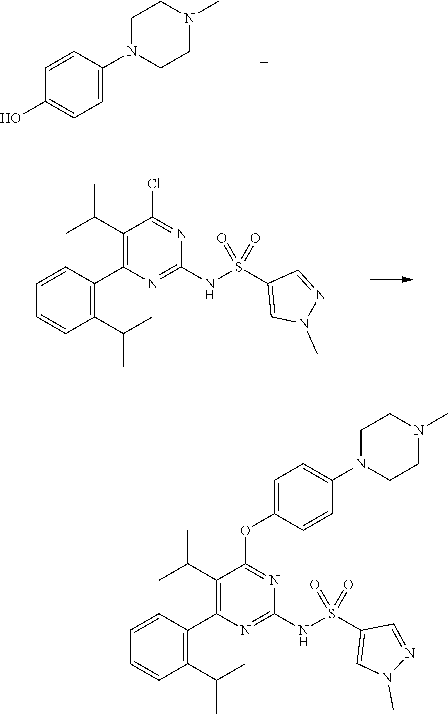 [SMILES: CN1CCN(c2ccc(O)cc2)CC1 ; CC(C)c1ccccc1-c1nc(NS(=O)(=O)c2cnn(C)c2)nc(Cl)c1C(C)C ; CC(C)c1ccccc1-c1nc(NS(=O)(=O)c2cnn(C)c2)nc(Oc2ccc(N3CCN(C)CC3)cc2)c1C(C)C]

A heterogeneous solution consisting of 4-(4-methylpiperazin-1-yl)phenol (approximately 14.42 mg, 0.07500 mmol), N-[4-chloro-5-isopropyl-6-(2-isopropylphenyl)pyrimidin-2-yl]-1-methyl-pyrazole-4-sulfonamide (10.85 mg, 0.0250 mmol), and potassium carbonate (approximately 13.82 mg, 0.1000 mmol) in NMP (125.0 μL) was heated in a sealed vial to 110° C. for 16 hours. The solution was acidified using acetic acid (approximately 45.04 mg, 42.65 μL, 0.7500 mmol), diluted with DMSO (0.5 mL), and filtered using a 0.2 μM PTFE syringe filter. The crude solution was separated by prep HPLC (gradient: 10 to 99% acetonitrile in water with 0.1% hydrochloric acid) which furnished the title compound as a white solid. N-[5-isopropyl-4-(2-isopropylphenyl)-6-[4-(4-methylpiperazin-1-yl)phenoxy]pyrimidin-2-yl]-1-methyl-pyrazole-4-sulfonamide (3.4 mg, 22%). ESI-MS m/z calc. 589.2835, found 590.59 (M+1)⁺; Retention time: 1.48 minutes; LC method A.

### Example 199: Preparation of Compound 540

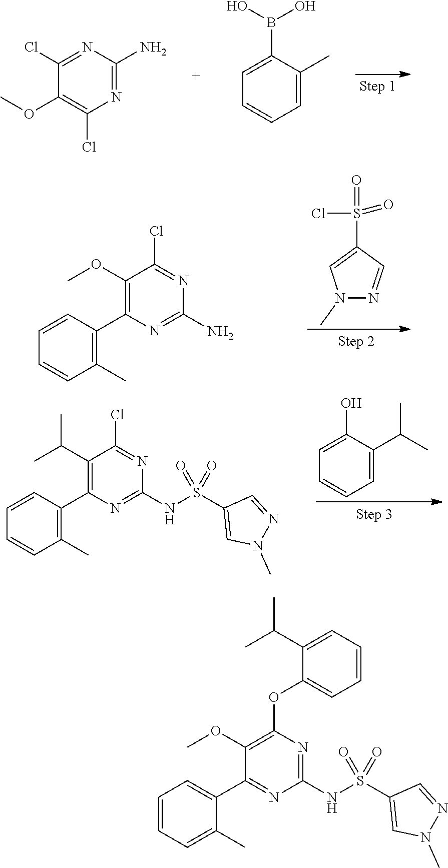

### Step 1: 4-Chloro-5-methoxy-6-(o-tolyl)pyrimidin-2-amine

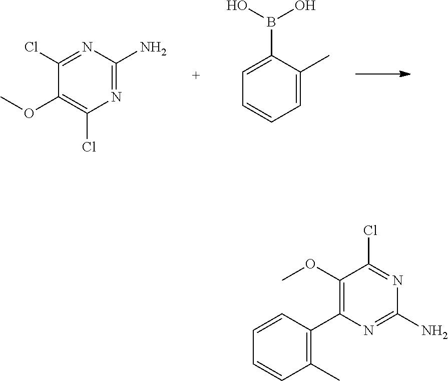

A heterogeneous mixture of 4,6-dichloro-5-methoxy-pyrimidin-2-amine (235 mg, 1.211 mmol), o-tolylboronic acid (approximately 164.6 mg, 1.211 mmol), tetrakis(triphenylphosphine)palladium(0) (approximately 279.9 mg, 0.2422 mmol), and potassium carbonate (approximately 502.1 mg, 3.633 mmol) in dioxane (4.846 mL) and water (968.2 μL) was sonicated for 2 minutes and sealed in a pressure tube. The reaction mixture was microwaved for 20 minutes at 120° C. The reaction solution was diluted with ethyl acetate and water. The organic layer was removed, and the aqueous layer was further extracted with ethyl acetate (4×). The combined organics were concentrated in vacuo on to silica gel. The crude impregnated silica gel was submitted to flash column chromatography on silica gel (20% ethyl acetate in hexanes) to give 4-chloro-5-methoxy-6-(o-tolyl)pyrimidin-2-amine (160 mg, 53%) as a white solid. ESI-MS m/z calc. 249.0669, found 250.2 (M+1)⁺; Retention time: 0.53 minutes (LC method D).

### Step 2: N-[4-chloro-5-methoxy-6-(o-tolyl)pyrimidin-2-yl]-1-methyl-pyrazole-4-sulfonamide

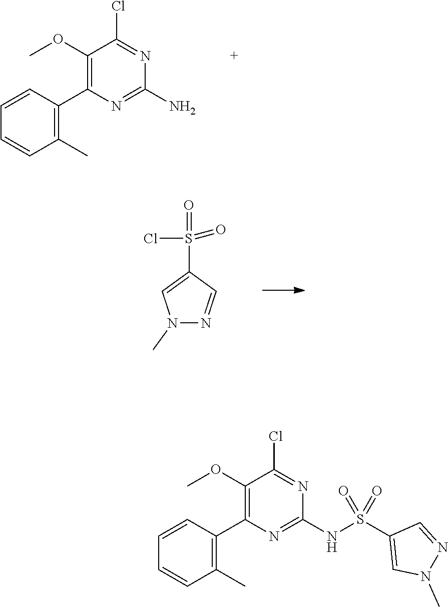

To a solution of 4-chloro-5-methoxy-6-(o-tolyl)pyrimidin-2-amine (160 mg, 0.6408 mmol) in DMF (3.238 mL) at 0° C. was added sodium hydride (approximately 61.51 mg, 2.563 mmol) and the reaction mixture was stirred at this temperature for 5 minutes, then removed from the cooling bath and stirred at room temperature for 10 minutes. The reaction mixture was cooled to 0° C. and a solution of 1-methylpyrazole-4-sulfonyl chloride (approximately 231.5 mg, 1.282 mmol) in DMF (3.0 mL) was added dropwise over 1 minutes. The reaction mixture was stirred at this temperature for 5 minutes, then removed from the cooling bath and stirred at room temperature for 12 minutes. The reaction mixture was cooled back to 0° C. and quenched with hydrochloric acid (approximately 284.2 μL of 37% w/v, 2.884 mmol), then diluted with a solution of ethyl acetate/hexanes (1:1) and partitioned with a saturated aqueous solution of sodium bicarbonate. The organic layer was removed, and the aqueous layer was further extracted with ethyl acetate/hexanes (1:1, 5×). The combined organic extracts were washed with brine and dried over magnesium sulfate. The solution was filtered, and the filtrate was concentrated in vacuo. The crude residue was purified by flash column chromatography on silica gel (5 to 40% ethyl acetate in hexanes). N-[4-chloro-5-methoxy-6-(o-tolyl)pyrimidin-2-yl]-1 -methyl-pyrazole-4-sulfonamide (130 mg, 52%) was isolated as a white solid. ESI-MS m/z calc. 393.06622, found 394.3 (M+1)⁺; Retention time: 0.58 minutes; LC method D.

### Step 3: N-[4-(2-isopropylphenoxy)-5-methoxy-6-(o-tolyl)pyrimidin-2-yl]-1-methyl-pyrazole-4-sulfonamide

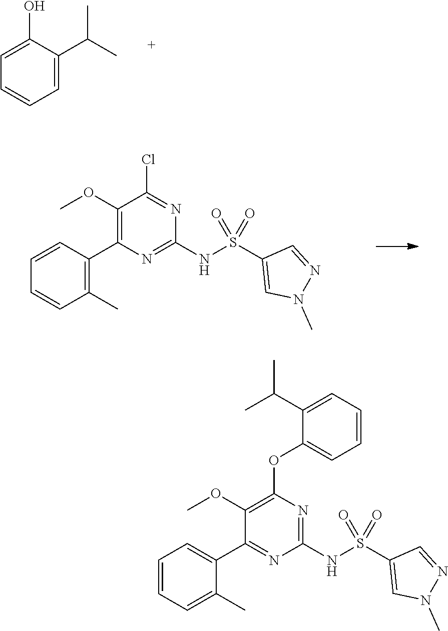

A heterogeneous mixture of 2-isopropylphenol (approximately 26.98 mg, 0.1981 mmol), N-[4-chloro-5-methoxy-6-(o-tolyl)pyrimidin-2-yl]-1-methyl-pyrazole-4-sulfonamide (26 mg, 0.06602 mmol), and cesium carbonate (approximately 107.6 mg, 0.3301 mmol) in NMP (264.1 μL) was heated in a sealed vial to 120° C. for 16 hours. Hydrochloric acid (approximately 52.05 μL of 37% w/v, 0.5282 mmol) and DMSO (0.50 mL) were added and then filtered. The samples were purified by HPLC (acetonitrile in water with 0.1% hydrochloric acid) to give N-[4-(2-isopropylphenoxy)-5-methoxy-6-(o-tolyl)pyrimidin-2-yl]-1-methyl-pyrazole-4-sulfonamide (21.9 mg, 67%). ESI-MS m/z calc. 493.17838, found 494.44 (M+1)⁺; Retention time: 1.96 minutes; LC method A.

### Example 200: Preparation of Compound 541

### Step 1: N-[5-methoxy-4-(o-tolyl)-6-phenoxy-pyrimidin-2-yl]-1-methyl-pyrazole-4-sulfonamide

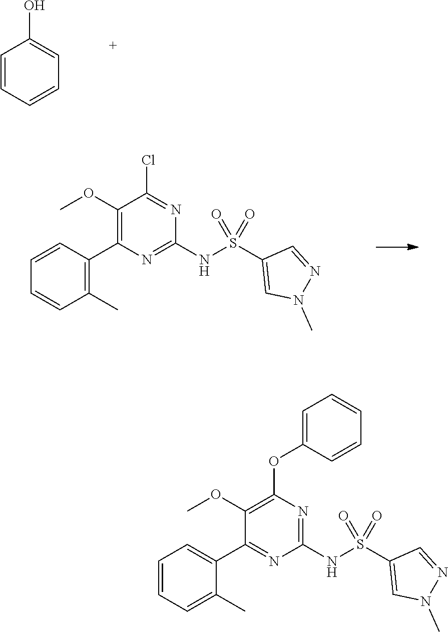 [SMILES: Oc1ccccc1 ; COc1c(Cl)nc(NS(=O)(=O)c2cnn(C)c2)nc1-c1ccccc1C ; COc1c(Oc2ccccc2)nc(NS(=O)(=O)c2cnn(C)c2)nc1-c1ccccc1C]

A heterogeneous mixture of phenol (approximately 18.64 mg, 17.58 μL, 0.1981 mmol), N-[4-chloro-5-methoxy-6-(o-tolyl)pyrimidin-2-yl]-1-methyl-pyrazole-4-sulfonamide (26 mg, 0.06602 mmol), and cesium carbonate (approximately 107.6 mg, 0.3301 mmol) in NMP (264.1 μL) was heated in a sealed vial to 120° C. for 16 hours. Hydrochloric acid (approximately 52.05 μL of 37% w/v, 0.5282 mmol) and DMSO (0.50 mL) were added and then filtered. The samples were purified by HPLC (acetonitrile in water with 0.1% hydrochloric acid). N-[5-methoxy-4-(o-tolyl)-6-phenoxy-pyrimidin-2-yl]-1-methyl-pyrazole-4-sulfonamide (9 mg, 30%). ESI-MS m/z calc. 451.13144, found 452.39 (M+1)⁺; Retention time: 1.68 minutes; LC method A.

### Example 201: Preparation of Compound 542

### Step 1: N-[5-methoxy-4-(2-methylphenoxy)-6-(o-tolyl)pyrimidin-2-yl]-1-methyl-pyrazole-4-sulfonamide

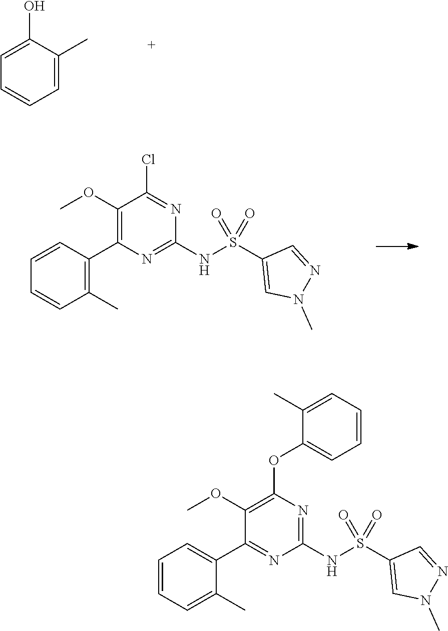

A heterogeneous mixture of o-cresol (approximately 21.42 mg, 38.76 μL, 0.1981 mmol), N-[4-chloro-5-methoxy-6-(o-tolyl)pyrimidin-2-yl]-1-methyl-pyrazole-4-sulfonamide (26 mg, 0.06602 mmol), and cesium carbonate (approximately 107.6 mg, 0.3301 mmol) in NMP (264.1 μL) was heated in a sealed vial to 120° C. for 16 hours. Hydrochloric acid (approximately 52.05 μL of 37% w/v, 0.5282 mmol) and DMSO (0.50 mL) were added and then filtered. The samples were purified by HPLC (acetonitrile in water with 0.1% hydrochloric acid) to give N-[5-methoxy-4-(2-methylphenoxy)-6-(o-tolyl)pyrimidin-2-yl]-1-methyl-pyrazole-4-sulfonamide (21.8 mg, 71%). ESI-MS m/z calc. 465.14706, found 466.4 (M+1)⁺; Retention time: 1.76 minutes; LC method A.

### Example 202: Preparation of Compound 543

### Step 1: N-[5-methoxy-4-(o-tolyl)-6-(4-piperazin-1-ylphenoxy)pyrimidin-2-yl]-1 -methyl-pyrazole-4-sulfonamide

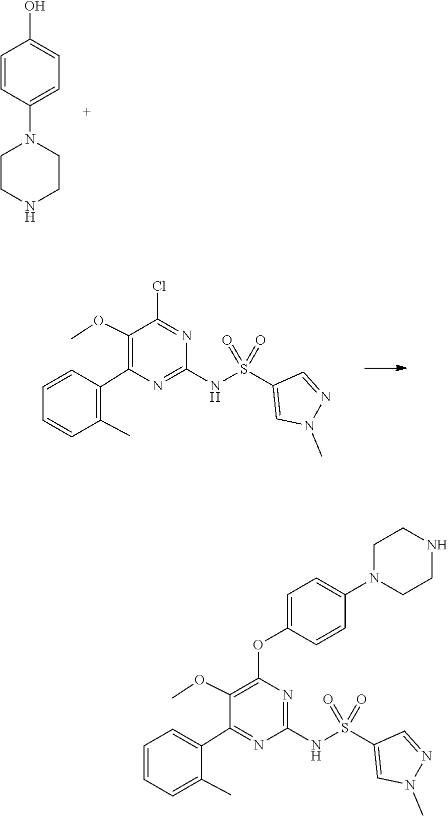 [SMILES: Oc1ccc(N2CCNCC2)cc1 ; COc1c(Cl)nc(NS(=O)(=O)c2cnn(C)c2)nc1-c1ccccc1C ; COc1c(Oc2ccc(N3CCNCC3)cc2)nc(NS(=O)(=O)c2cnn(C)c2)nc1-c1ccccc1C]

A heterogeneous mixture of 4-piperazin-1-ylphenol (approximately 35.31 mg, 0.1981 mmol), N-[4-chloro-5-methoxy-6-(o-tolyl)pyrimidin-2-yl]-1-methyl-pyrazole-4-sulfonamide (26 mg, 0.06602 mmol), and cesium carbonate (approximately 107.6 mg, 0.3301 mmol) in NMP (264.1 μL) was heated in a sealed vial to 120° C. for 16 hours. Hydrochloric acid (approximately 52.05 μL of 37% w/v, 0.5282 mmol) and DMSO (0.50 mL) were added and then filtered. The sample was purified by HPLC (acetonitrile in water with 0.1% hydrochloric acid) to give N-[5-methoxy-4-(o-tolyl)-6-(4-piperazin-1-ylphenoxy)pyrimidin-2-yl]-1-methyl-pyrazole-4-sulfonamide (4.4 mg, 12%). ESI-MS m/z calc. 535.2002, found 536.5 (M+1)⁺; Retention time: 1.21 minutes; LC method A.

### Example 203: Characterization of Compounds 544-555

The compounds in the following tables were prepared in a manner analogous to that described above using commercially available reagents and intermediates described herein.

| Compound | | LCMS | Calc. | | LCMS |
|---|---|---|---|---|---|
| number | Structure | Rt (min) | mass | M + 1 | Method |
| 544 | 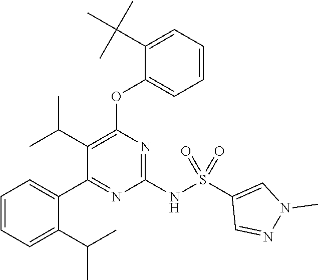 [SMILES: CC(C)c1ccccc1-c1nc(NS(=O)(=O)c2cnn(C)c2)nc(Oc2ccccc2C(C)(C)C)c1C(C)C] | 2.38 | 547.262 | 548.58 | A |
| 545 | 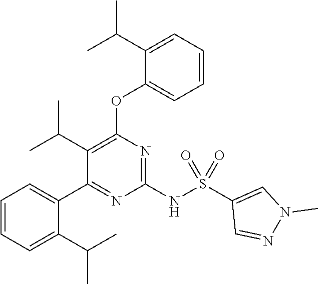 [SMILES: CC(C)c1ccccc1Oc1nc(NS(=O)(=O)c2cnn(C)c2)nc(-c2ccccc2C(C)C)c1C(C)C] | 2.29 | 533.246 | 534.57 | A |
| 546 | 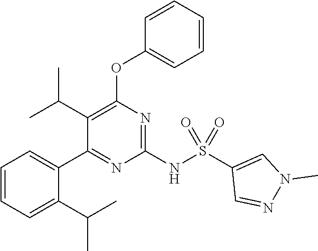 [SMILES: CC(C)c1ccccc1-c1nc(NS(=O)(=O)c2cnn(C)c2)nc(Oc2ccccc2)c1C(C)C] | 2.05 | 491.199 | 492.52 | A |
| 547 | 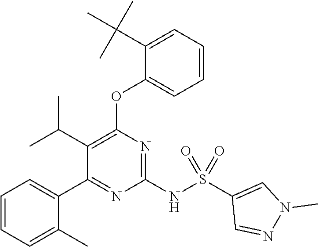 [SMILES: Cc1ccccc1-c1nc(NS(=O)(=O)c2cnn(C)c2)nc(Oc2ccccc2C(C)(C)C)c1C(C)C] | 2.21 | 519.23 | 520.56 | A |
| 548 | 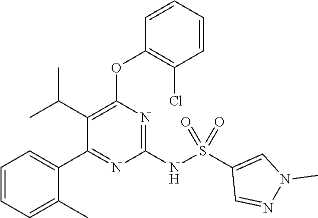 [SMILES: Cc1ccccc1-c1nc(NS(=O)(=O)c2cnn(C)c2)nc(Oc2ccccc2Cl)c1C(C)C] | 1.99 | 497.129 | 498.44 | A |
| 549 | 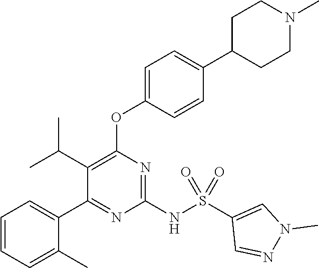 [SMILES: Cc1ccccc1-c1nc(NS(=O)(=O)c2cnn(C)c2)nc(Oc2ccc(C3CCN(C)CC3)cc2)c1C(C)C] | 1.35 | 560.257 | 561.6 | A |
| 550 | 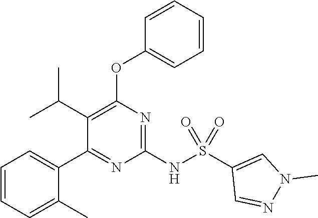 [SMILES: Cc1ccccc1-c1nc(NS(=O)(=O)c2cnn(C)c2)nc(Oc2ccccc2)c1C(C)C] | 1.85 | 463.168 | 464.47 | A |
| 551 | 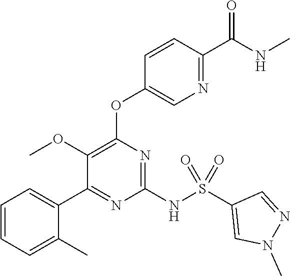 [SMILES: CNC(=O)c1ccc(Oc2nc(NS(=O)(=O)c3cnn(C)c3)nc(-c3ccccc3C)c2OC)cn1] | 1.42 | 509.148 | 510.45 | A |
| 552 | 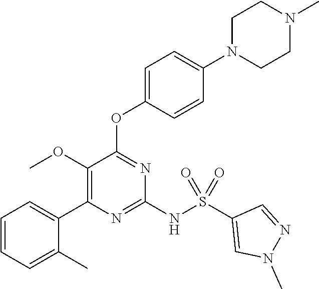 [SMILES: COc1c(Oc2ccc(N3CCN(C)CC3)cc2)nc(NS(=O)(=O)c2cnn(C)c2)nc1-c1ccccc1C] | 1.13 | 549.216 | 550 | A |
| 553 | 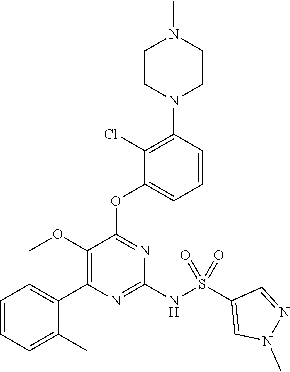 [SMILES: COc1c(Oc2cccc(N3CCN(C)CC3)c2Cl)nc(NS(=O)(=O)c2cnn(C)c2)nc1-c1ccccc1C] | 1.21 | 583.177 | 584 | A |
| 554 | 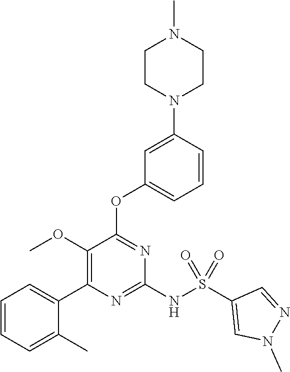 [SMILES: COc1c(Oc2cccc(N3CCN(C)CC3)c2)nc(NS(=O)(=O)c2cnn(C)c2)nc1-c1ccccc1C] | 1.16 | 549.216 | 549 | A |
| 555 | 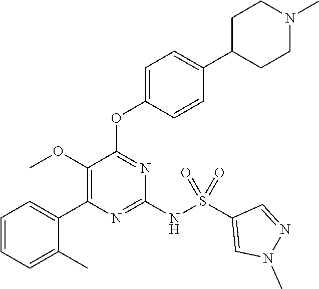 [SMILES: COc1c(Oc2ccc(C3CCN(C)CC3)cc2)nc(NS(=O)(=O)c2cnn(C)c2)nc1-c1ccccc1C] | 1.16 | 548.221 | 549 | A |

| Compound | |
|---|---|
| number | NMR |
| 554 | ¹H NMR (400 MHz, DMSO-d₆) δ 11.45 (s, 1H), 10.37 (s, 1H), 7.44 (s, |
| | 2H), 7.36 (d, J = 16.5 Hz, 4H), 7.14 (s, 1H), 7.04 (s,2H), 6.83 (d, J = |
| | 7.6 Hz, 1H), 3.95 (d, J = 8.5 Hz, 2H), 3.73 (s, 3H), 3.58 (s, 3H), 3.50 (s, |
| | 2H), 3.29 (s, 2H), 3.15 (s, 4H), 2.83 (s, 3H), 2.19 (s, 4H). |
| 555 | ¹H NMR (400 MHz, DMSO-d₆) δ 11.36 (s, 1H), 10.31 (s, 1H), 7.53 (s, |
| | 1H), 7.41 (s, 2H), 7.39 − 7.31 (m, 4H), 7.30 (s, 2H), 7.03 (s, 1H), 3.74 |
| | (s, 3H), 3.58 (s, 3H), 3.49 (d, J = 11.6 Hz, 2H), 3.07 (s, 2H), 2.90 (s, |
| | 1H), 2.78 (d, J = 2.0 Hz, 3H), 2.18 (s, 3H), 2.02 (s, 3H). |

### Example 204: Preparation of Compound 556

### Step 1: N-[5-isopropoxy-4-[4-(1-methyl-4-piperidyl)phenoxy]-6-(o-tolyl)pyrimidin-2-yl]-1-methyl-pyrazole-4-sulfonamide

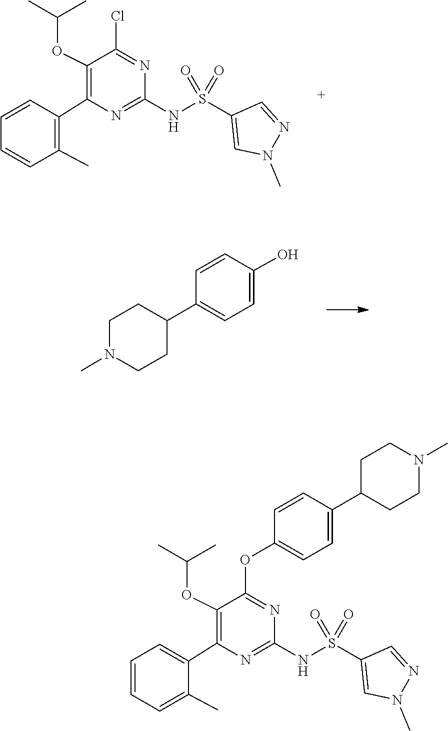 [SMILES: Cc1ccccc1-c1nc(NS(=O)(=O)c2cnn(C)c2)nc(Cl)c1OC(C)C ; CN1CCC(c2ccc(O)cc2)CC1 ; Cc1ccccc1-c1nc(NS(=O)(=O)c2cnn(C)c2)nc(Oc2ccc(C3CCN(C)CC3)cc2)c1OC(C)C]

An mixture of N-[4-chloro-5-isopropoxy-6-(o-tolyl)pyrimidin-2-yl]-1-methyl-pyrazole-4-sulfonamide (50 mg, 0.1185 mmol), 4-(1-methyl-4-piperidyl)phenol (approximately 29.46 mg, 0.1540 mmol), and cesium carbonate (approximately 154.4 mg, 0.4740 mmol) in NMR was stirred at 110° C. for 16 hours and then cooled to room temperature. The solution was filtered and purified by reverse-phase preparative HPLC using a 15 minutes gradient of 1% MeCN in water to 99% MeCN (HCl modifier) to give N-[5-isopropoxy-4-[4-(1-methyl-4-piperidyl)phenoxy]-6-(o-tolyl)pyrimidin-2-yl]-1-methyl-pyrazole-4-sulfonamide (31.5 mg, 46%) ESI-MS m/z calc. 576.2519, found 577.0 (M+1)⁺; Retention time: 1.33 minutes; LC method A. H NMR (400 MHz, DMSO-d₆) δ 10.70 (s, 1H), 7.54 (s, 1H), 7.40 (d, J=8.5 Hz, 3H), 7.31 (s, 5H), 7.07 (s, 1H), 3.94 (s, 1H), 3.74 (s, 3H), 3.49 (d, J=11.5 Hz, 2H), 3.08 (s, 2H), 2.89 (s, 1H), 2.76 (d, J=4.7 Hz, 3H), 2.22 (s, 3H), 2.03 (s, 4H), 0.94 (d, J=6.1 Hz, 6H).

### Example 205: Preparation of Compound 557

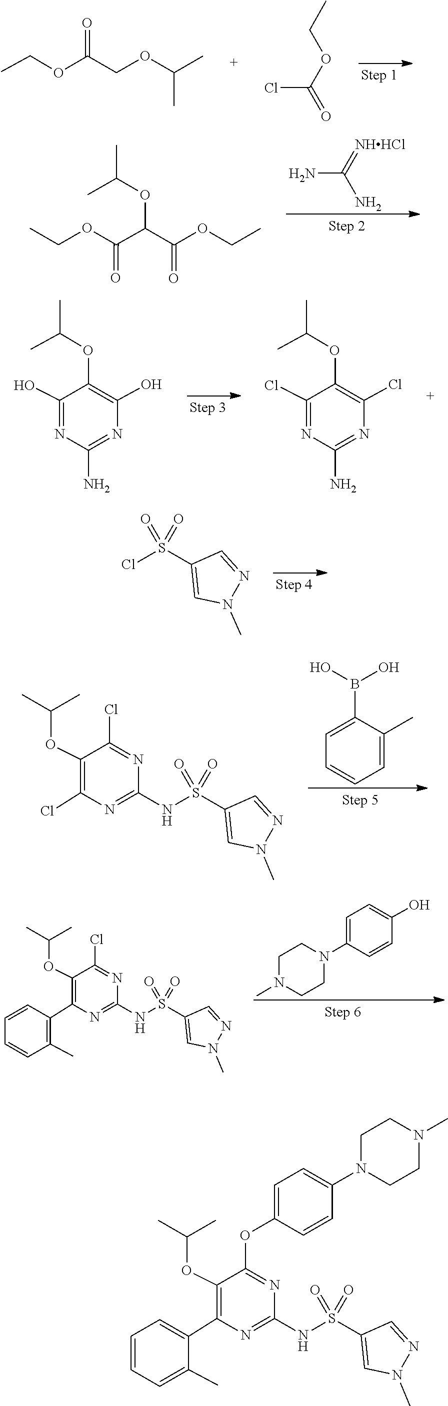

### Step 1: Diethyl 2-isopropoxypropanedioate

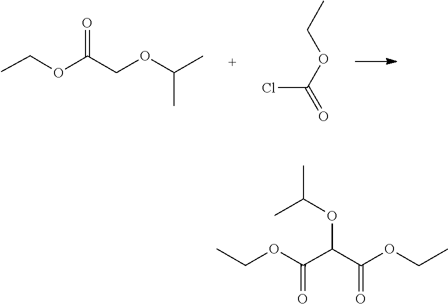

To a solution of diisopropylamine (11.119 g, 15.4 mL, 109.88 mmol) in THF (100 mL) at −78° C. was added n-butyllithium (41.3 mL of 2.5 M, 103.25 mmol). The mixture was stirred at 0° C. for 15 minutes and cooled to −78° C., a solution of ethyl 2-isopropoxyacetate (10.83 g, 68.899 mmol) in THE (25 mL) was added dropwise at a temperature below −74° C. The mixture was stirred for 10 minutes. A solution of ethyl chloroformate (7.9450 g, 7 mL, 73.210 mmol) in THE (15 mL) was added dropwise at −73° C. The mixture was stirred for 10 minutes and allowed to warm up to −50° C. and stirred for 5 minutes. Solid ammonium chloride (7.37 g, 2 eq) was added. The mixture was stirred at −70° C. for 5 minutes and crushed ice (20 g) was added. The mixture was allowed to warm up to room temperature and the organic layer was separated and concentrated after drying over sodium sulfate. The yellow oil residue was purified by flash chromatography on silica gel (220 g) using a 0-25% gradient of ethyl acetate in heptanes over 22 column volumes to give the title compound diethyl 2-isopropoxypropanedioate (9.26 g, 55% yield) as a yellow oil. ¹H NMR (300 MHz, CDCl₃) δ ppm 1.24 (d, J=5.9 Hz, 6H), 1.29 (t, J=7.2 Hz, 6H), 3.69-3.83 (m, 1H), 4.19-4.33 (m, 4H), 4.54 (s, 1H). ESI-MS m/z calc. 218.12, found 241.1 (M+Na)*. Retention time 1.78 minutes

### Step 2: 2-Amino-5-isopropoxy-pyrimidine-4,6-diol

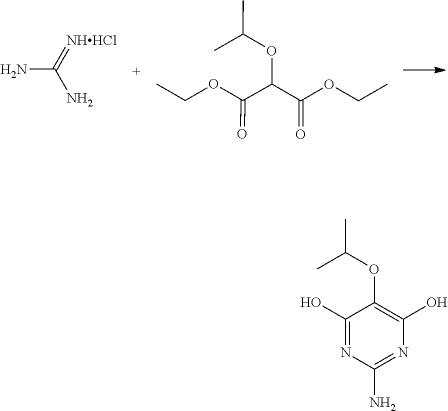

To a 1000-mL three neck flask was added absolute ethanol (215 mL) and solid chunks of sodium metal (4.13 g, 179.64 mmol) were added gradually and the mixture was stirred until all solids dissolved. Once cooled back to around 25° C., guanidine (hydrochloride salt) (6.3 g, 65.947 mmol) and diethyl 2-isopropoxypropanedioate (13.33 g, 59.856 mmol) were successively added. An additional amount of ethanol (70 mL) was added and the reaction was stirred at reflux overnight. Once cooled to room temperature, the crude mixture was concentrated under reduced pressure to remove most of the ethanol. Water (220 mL) was added until dissolution, the resulting solution was cooled in an ice bath and acidified to pH of 5 using acetic acid (120-140 mL). The solids were filtered and washed with water. The solid was dried under reduced pressure and under high vacuum to give 2-amino-5-isopropoxy-pyrimidine-4,6-diol hydrate (7.125 g, 54% yield) as a light pink solid. ¹H NMR (300 MHz, DMSO-d₆) δ ppm 1.08 (d, J=6.2 Hz, 6H), 4.01-4.14 (m, 1H), 6.81 (br. s, 2H), 10.82 (br. s, 2H). ESI-MS m/z calc. 185.08, found 186.1 (M+1)⁺; Retention time: 0.41 minutes (LC method C).

### Step 3: 4,6-Dichloro-5-isopropoxy-pyrimidin-2-amine

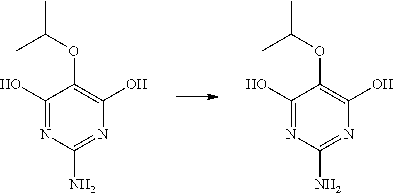

A vigorously stirred suspension of 2-amino-5-isopropoxy-pyrimidine-4,6-diol (7.12 g, 35.508 mmol) in phosphorus oxychloride (53.627 g, 32.6 mL, 349.75 mmol) was heated to 90° C. and N,N-diethylaniline (7.4400 g, 8 mL, 49.855 mmol) was added dropwise. The suspension was stirred for one to three hours at 105° C., then upon completion was allowed to cool to room temperature. Excess phosphorus oxychloride was quenched by careful addition of 450 mL of aqueous 1.0 N NaOH, while keeping internal temperature below 60° C. At the end of the addition the pH reached about 1-2, then it was adjusted to pH=5 by careful addition of a 25% aqueous solution of NaOH. The resultant mixture was added to an extraction funnel and extracted with dichloromethane (3×120 mL). The combined organic phases were washed with brine (1×80 mL), then dried by filtration over a cotton pad with anhydrous sodium sulfate, then concentrated by rotary evaporation. The residue was purified by flash chromatography using a gradient of ethyl acetate (0-30%) in heptanes on a 120 g column (silica gel) to give 4,6-dichloro-5-isopropoxy-pyrimidin-2-amine (3.97 g, 50%). ¹H NMR (300 MHz, CDCl₃) δ ppm 1.36 (d, J=6.2 Hz, 6H), 4.42 (dq, J=12.3, 6.2 Hz, 1H), 5.07 (br. s, 2H). ESI-MS m/z calc. 221.0123, found 222.1 (M+1)⁺; Retention time: 1.77 minutes (LC method P).

### Step 4: N-(4,6-Dichloro-5-isopropoxy-pyrimidin-2-yl)-1-methyl-pyrazole-4-sulfonamide

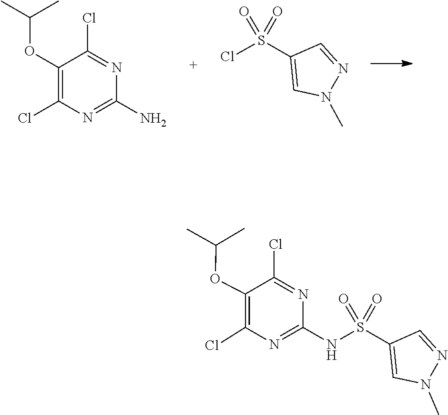

A DMF (30 mL) suspension of NaH (797.7 mg, 33.24 mmol) was cooled to 0° C. using an ice-water bath and it was treated with solid 4,6-dichloro-5-isopropoxy-pyrimidin-2-amine (2 g, 9.006 mmol). The reaction mixture was stirred for 20 minutes and then treated with solid 1-methylpyrazole-4-sulfonyl chloride (1.8 g, 9.966 mmol). The reaction mixture was warmed to room temperature and stirred for 1 hour and then cooled to 0° C. Aqueous HCl (27 mL of 1 M, 27.00 mmol) was added and the reaction mixture was stirred at 0° C. for 10 minutes upon which the product crystallized out. The reaction mixture was filtered, rinsed with cold water, and dried overnight to give N-(4,6-dichloro-5-isopropoxy-pyrimidin-2-yl)-1-methyl-pyrazole-4-sulfonamide (3.24 g, 97%). ¹H NMR (400 MHz, DMSO-d₆) δ 12.16 (s, 1H), 8.39 (s, 1H), 7.90 (s, 1H), 4.46 (s, 1H), 3.88 (s, 3H), 1.30 (d, J=6.1 Hz, 7H). ESI-MS m/z calc. 365.01163, found 366.0 (M+1)⁺; Retention time: 1.36 minutes (LC method A).

### Step 5: N-[4-chloro-5-isopropoxy-6-(o-tolyl)pyrimidin-2-yl]-1-methyl-pyrazole-4-sulfonamide

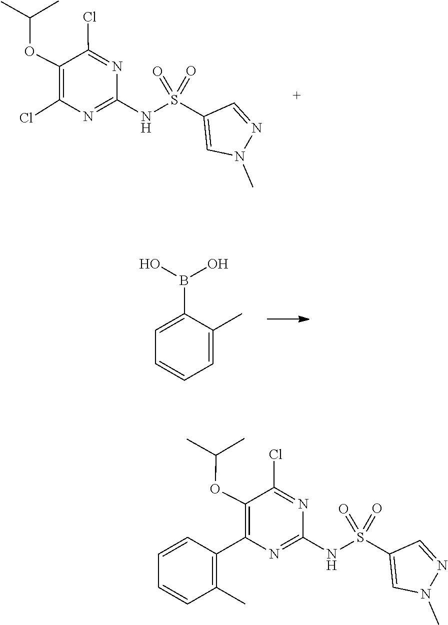

N-(4,6-dichloro-5-isopropoxy-pyrimidin-2-yl)-1-methyl-pyrazole-4-sulfonamide (1.4 g, 3.785 mmol), o-tolylboronic acid (540 mg, 3.972 mmol), tetrakis(triphenylphosphine)palladium (0) (218 mg, 0.1887 mmol), 2 M sodium carbonate (6 mL of 2 M, 12.00 mmol) and 1,2-dimethoxyethane (15 mL) were combined in a microwave vial. The mixture was purged with nitrogen, sealed and stirred for 65 hours at 70° C. The reaction mixture was diluted with water, extracted with ethyl acetate. The organic layer was dried over Na₂SO₄, filtered, concentrated, and purified on silica using a gradient of ethyl acetate/hexane. The product from silica column was purified on reverse phase HPLC (HCl modifier, 25-75% ACN-H₂O) to give N-[4-chloro-5-isopropoxy-6-(o-tolyl)pyrimidin-2-yl]-1-methyl-pyrazole-4-sulfonamide (684.0 mg, 43%). ¹H NMR (400 MHz, DMSO-d₆) δ 11.88 (s, 1H), 8.27 (s, 1H), 7.77 (s, 1H), 7.36 (d, J=29.2 Hz, 4H), 3.80 (d, J=24.9 Hz, 4H), 2.18 (s, 3H), 0.91 (s, 6H). ESI-MS m/z calc. 421.09753, found 422.4 (M+1)⁺; Retention time: 1.67 minutes (LC method A).

### Step 6: N-[5-isopropoxy-4-[4-(4-methylpiperazin-1-yl)phenoxy]-6-(o-tolyl)pyrimidin-2-yl]-1-methyl-pyrazole-4-sulfonamide

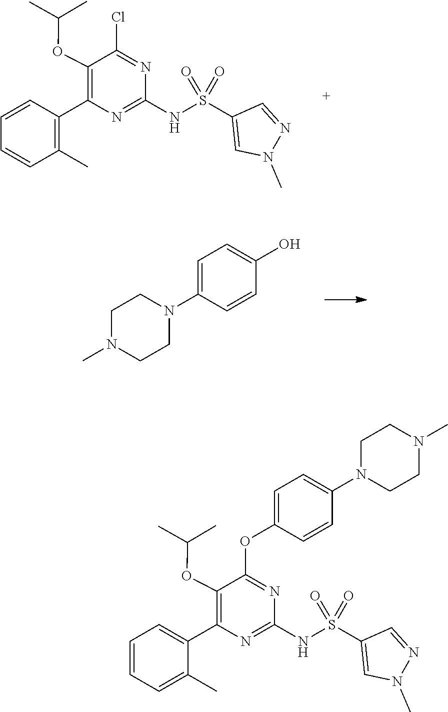 [SMILES: Cc1ccccc1-c1nc(NS(=O)(=O)c2cnn(C)c2)nc(Cl)c1OC(C)C ; CN1CCN(c2ccc(O)cc2)CC1 ; Cc1ccccc1-c1nc(NS(=O)(=O)c2cnn(C)c2)nc(Oc2ccc(N3CCN(C)CC3)cc2)c1OC(C)C]

A mixture of N-[4-chloro-5-isopropoxy-6-(o-tolyl)pyrimidin-2-yl]-1-methyl-pyrazole-4-sulfonamide (50 mg, 0.1185 mmol), 4-(4-methylpiperazin-1-yl)phenol (approximately 29.61 mg, 0.1540 mmol), and cesium carbonate (approximately 154.4 mg, 0.4740 mmol) in NMP was stirred at 110° C. for 16 hours and then cooled to room temperature. The solution was filtered and purified by reverse-phase preparative HPLC using a 15 minutes gradient of 1% MeCN in water to 99% MeCN (HCl modifier) to give N-[5-isopropoxy-4-[4-(4-methylpiperazin-1-yl)phenoxy]-6-(o-tolyl)pyrimidin-2-yl]-1-methyl-pyrazole-4-sulfonamide (34.6 mg, 50%) ESI-MS m/z calc. 577.24713, found 578.0 (M+1)⁺; Retention time: 1.31 minutes; LC method A. ¹H NMR (400 MHz, DMSO-d₆) δ 10.95 (s, 1H), 7.55 (s, 1H), 7.40-7.35 (m, 1H), 7.32 (d, J=11.4 Hz, 3H), 7.23 (d, J=9.1 Hz, 2H), 7.20 (s, 1H), 7.15 (d, J=9.2 Hz, 2H), 3.96 (s, 1H), 3.85 (d, J=10.4 Hz, 2H), 3.75 (s, 3H), 3.50 (d, J=9.9 Hz, 2H), 3.16 (s, 4H), 2.81 (d, J=4.6 Hz, 3H), 2.21 (s, 3H), 0.94 (d, J=6.1 Hz, 6H).

### Example 206: Preparation of Compound 558

### Step 1: N-[5-isopropoxy-4-[3-(4-methylpiperazin-1-yl)phenoxy]-6-(o-tolyl)pyrimidin-2-yl]-1-methyl-pyrazole-4-sulfonamide

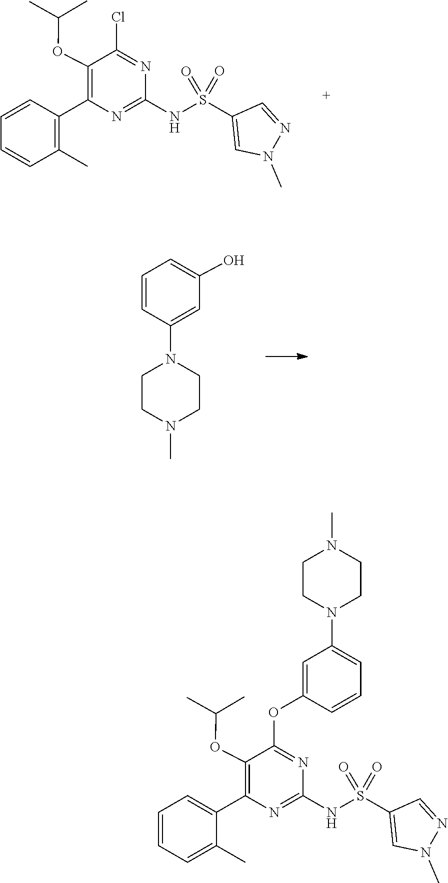 [SMILES: Cc1ccccc1-c1nc(NS(=O)(=O)c2cnn(C)c2)nc(Cl)c1OC(C)C ; CN1CCN(c2cccc(O)c2)CC1 ; Cc1ccccc1-c1nc(NS(=O)(=O)c2cnn(C)c2)nc(Oc2cccc(N3CCN(C)CC3)c2)c1OC(C)C]

A mixture of N-[4-chloro-5-isopropoxy-6-(o-tolyl)pyrimidin-2-yl]-1-methyl-pyrazole-4-sulfonamide (50 mg, 0.1185 mmol), 3-(4-methylpiperazin-1-yl)phenol (approximately 30.84 mg, 0.1540 mmol), and cesium carbonate (approximately 154.4 mg, 0.4740 mmol) in NMP was stirred at 110° C. for 16 hours and then cooled to room temperature. The solution was filtered and purified by reverse phase preparative HPLC using a 15 minutes gradient of 1% MeCN in water to 99% MeCN (HCl modifier) to give N-[5-isopropoxy-4-[3-(4-methylpiperazin-1-yl)phenoxy]-6-(o-tolyl)pyrimidin-2-yl]-1-methyl-pyrazole-4-sulfonamide (29.2 mg, 43%). ESI-MS m/z calc. 577.24713, found 578.0 (M+1)⁺; Retention time: 1.32 minutes; LC method A. 1H NMR (400 MHz, DMSO-d₆) δ 11.15 (s, 1H), 7.48 (s, 1H), 7.44-7.29 (m, 6H), 7.17 (s, 1H), 7.01 (d, J=8.7 Hz, 2H), 6.79 (d, J=8.0 Hz, 1H), 3.99-3.92 (m, 4H), 3.74 (s, 3H), 3.47 (d, J=11.7 Hz, 2H), 3.27-3.12 (m, 4H), 2.80 (d, J=4.6 Hz, 3H), 2.22 (s, 3H), 0.95 (d, J=6.2 Hz, 6H).

### Example 207: Preparation of Compound 559

### Step 1: N-[5-isopropoxy-4-(2-methylphenoxy)-6-(o-tolyl)pyrimidin-2-yl]-1-methyl-pyrazole-4-sulfonamide

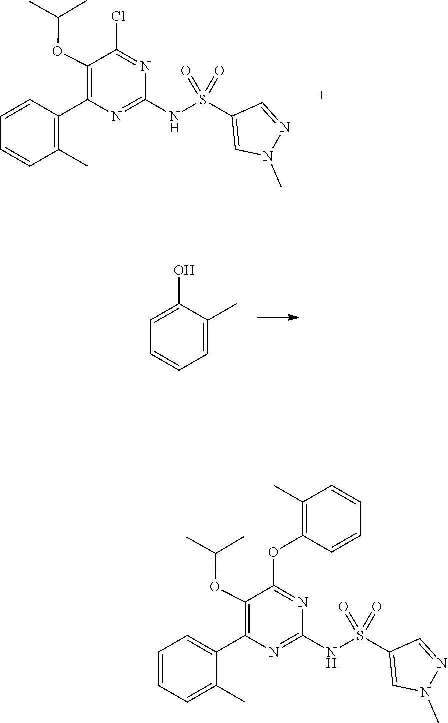

A mixture of N-[4-chloro-5-isopropoxy-6-(o-tolyl)pyrimidin-2-yl]-1-methyl-pyrazole-4-sulfonamide (50 mg, 0.1185 mmol), o-cresol (approximately 16.65 mg, 30.12 μL, 0.1540 mmol), and cesium carbonate (approximately 154.4 mg, 0.4740 mmol) in NMP was stirred at 110° C. for 16 hours and then cooled to room temperature. The solution was filtered and purified by reverse phase preparative HPLC using a 15 minutes gradient of 1% MeCN in water to 99% MeCN (HCl modifier) to give N-[5-isopropoxy-4-(2-methylphenoxy)-6-(o-tolyl)pyrimidin-2-yl]-1-methyl-pyrazole-4-sulfonamide (54.8 mg, 94%). ESI-MS m/z calc. 493.17838, found 494.0 (M+1)⁺; Retention time: 1.9 minutes; LC method A. ¹H NMR (400 MHz, DMSO-d₆) δ 11.39 (s, 1H), 7.44 (d, J=6.5 Hz, 1H), 7.40-7.27 (m, 7H), 7.23 (d, J=7.9 Hz, 1H), 7.08 (s, 1H), 3.99 (s, 1H), 3.74 (s, 3H), 2.23 (s, 3H), 2.18 (s, 3H), 0.96 (d, J=6.1 Hz, 6H).

### Example 208: Preparation of Compound 560

### Step 1: 2-chloro-3-(4-methylpiperazin-1-yl)phenol

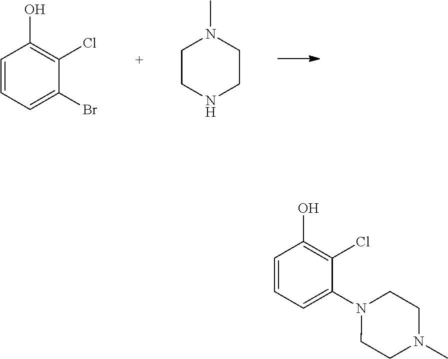

A heterogeneous mixture of 3-bromo-2-chloro-phenol (4.20 g, 20.25 mmol), 1-methylpiperazine (21.5 g, 214.7 mmol), chloro(2-di-t-butylphosphino-2′, 4′, 6′-tri-i-propyl-1,1′-biphenyl)[2-(2-aminoethyl)phenyl] palladium(II) [t-BuXPhos Palladacycle Gen. 1] (2.1 g, 3.058 mmol), and potassium tert-butoxide (4.8 g, 42.78 mmol) in dioxane (120 mL) was sonicated for 15 minutes and heated in a sealed vessel at 50° C. for 16 hours. The reaction mixture was acidified using acetic acid (3.4 mL, 59.79 mmol) then partitioned between DCM (100 mL) and water (100 mL). The organic layer was separated, and the aqueous layer was further extracted with DCM (4×). The combined organics were washed once with brine, dried using magnesium sulfate, filtered, and concentrated in vacuo. The crude residue was separated by flash column chromatography on silica gel (gradient: 1 to 10% methanol in dichloromethane) to afford 2-chloro-3-(4-methylpiperazin-1-yl)phenol (3.86 g, 84%) as a yellow solid. ESI-MS m/z calc. 226.0873, found 227.12 (M+1)⁺; Retention time: 0.24 minutes; LC method D.

### Step 2: N-[4-[2-chloro-3-(4-methylpiperazin-1-yl)phenoxy]-5-isopropoxy-6-(o-tolyl)pyrimidin-2-yl]-1-methyl-pyrazole-4-sulfonamide

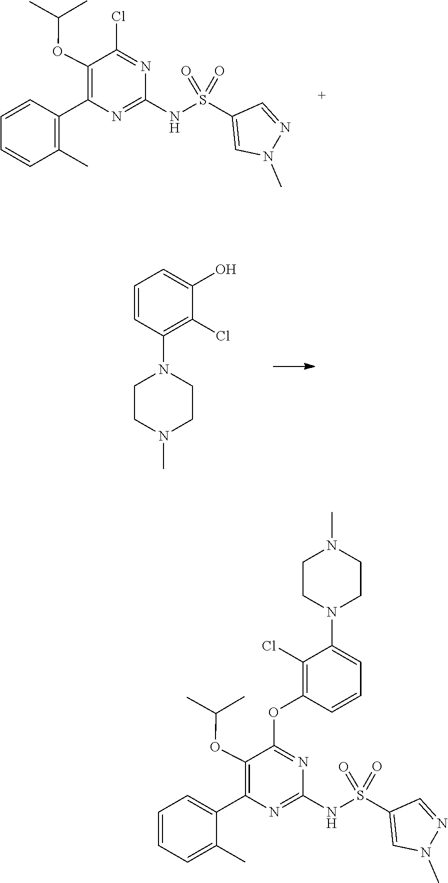 [SMILES: Cc1ccccc1-c1nc(NS(=O)(=O)c2cnn(C)c2)nc(Cl)c1OC(C)C ; CN1CCN(c2cccc(O)c2Cl)CC1 ; Cc1ccccc1-c1nc(NS(=O)(=O)c2cnn(C)c2)nc(Oc2cccc(N3CCN(C)CC3)c2Cl)c1OC(C)C]

A mixture of N-[4-chloro-5-isopropoxy-6-(o-tolyl)pyrimidin-2-yl]-1-methyl-pyrazole-4-sulfonamide (50 mg, 0.1185 mmol), 2-chloro-3-(4-methylpiperazin-1-yl)phenol (approximately 34.91 mg, 0.1540 mmol), and cesium carbonate (approximately 154.4 mg, 0.4740 mmol) in NMP was stirred at 110° C. for 16 hours and then cooled to room temperature. The solution was filtered and purified by reverse phase preparative HPLC using a 15 minutes gradient of 1% MeCN in water to 99% MeCN (HCl modifier) to give N-[4-[2-chloro-3-(4-methylpiperazin-1-yl)phenoxy]-5-isopropoxy-6-(o-tolyl)pyrimidin-2-yl]-1-methyl-pyrazole-4-sulfonamide (14.0 mg, 19%). ESI-MS m/z calc. 611.2081, found 612.0 (M+1)⁺; Retention time: 1.37 minutes; LC method A. ¹H NMR (400 MHz, DMSO-d₆) δ 11.44 (s, 1H), 10.78 (s, 1H), 7.58 (s, 1H), 7.51 (s, 1H), 7.39 (s, 1H), 7.33 (d, J=7.7 Hz, 3H), 7.25 (s, 2H), 7.03 (s, 1H), 4.05 (s, 1H), 3.78 (s, 3H), 3.54 (s, 4H), 3.18 (d, J=11.0 Hz, 4H), 2.84 (s, 3H), 2.22 (s, 3H), 0.96 (d, J=6.1 Hz, 6H).

### Example 209: Preparation of Compound 561

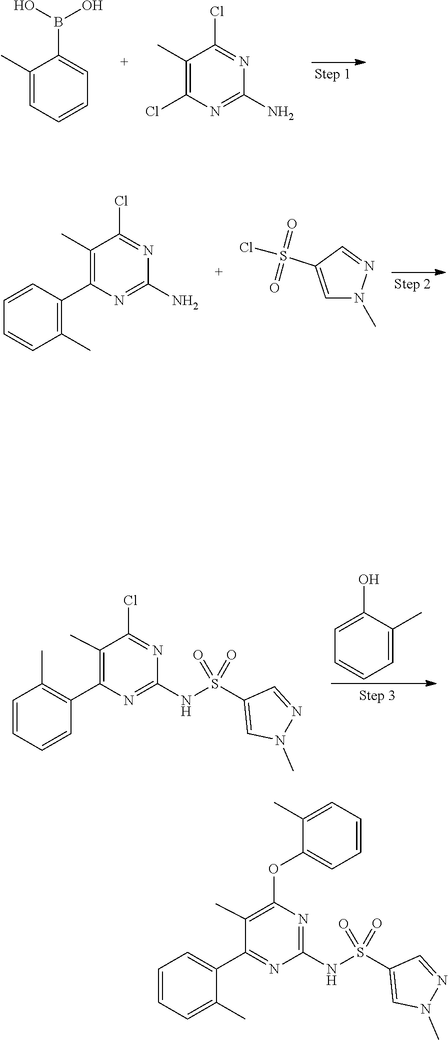

### Step 1: 4-chloro-5-methyl-6-(o-tolyl)pyrimidin-2-amine

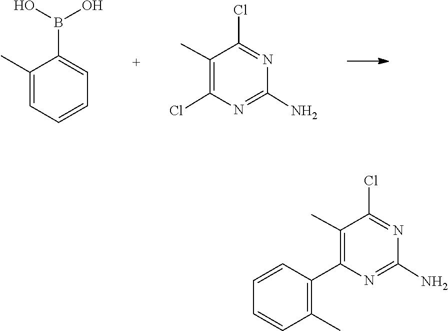

A heterogeneous solution of o-tolylboronic acid (approximately 704.9 mg, 5.185 mmol), 4,6-dichloro-5-methyl-pyrimidin-2-amine (0.923 g, 5.185 mmol), bis(triphenylphosphine)palladium(II) dichloride (approximately 109.2 mg, 0.1556 mmol), and potassium carbonate (approximately 1.433 g, 10.37 mmol) in 1,4-dioxane (11.33 mL) was sealed in a pressure vessel and heated to 80° C. for 16 hours. The reaction mixture was diluted with dichloromethane and a saturated solution of aqueous ammonium chloride was added. The organic layer was removed, and the aqueous layer was further extracted with dichloromethane (3×). The combined organics were dried over magnesium sulfate, filtered, and concentrated. The crude mixture was separated by flash column chromatography on silica gel (25% ethyl acetate in hexanes). 4-chloro-5-methyl-6-(o-tolyl)pyrimidin-2-amine (710 mg, 50%) was isolated as a yellow crystalline solid. ESI-MS m/z calc. 233.07198, found 234.19 (M+1)⁺; Retention time: 0.55 minutes (LC method D).

### Step 2: N-[4-chloro-5-methyl-6-(2-methylphenyl)pyrimidin-2-yl]-1-methyl-1H-pyrazole-4-sulfonamide

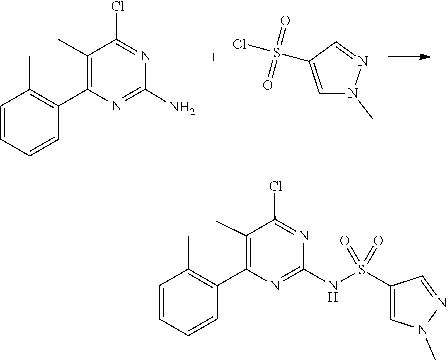

To a solution of 4-chloro-5-methyl-6-(o-tolyl)pyrimidin-2-amine (710 mg, 3.038 mmol) in DMF (12.15 mL) at 0° C. was added NaH (approximately 291.6 mg, 12.15 mmol) and the reaction mixture was stirred at this temperature for 5 minutes, then removed from the cooling bath and stirred at room temperature for 10 minutes. The reaction mixture was cooled to 0° C. and 1-methylpyrazole-4-sulfonyl chloride (approximately 1.097 g, 6.076 mmol) was added dropwise. The reaction mixture was stirred at this temperature for 5 minutes, then removed from the cooling bath and stirred at room temperature for 12 minutes. The reaction mixture was cooled back to 0° C. and quenched with HCl (approximately 1.347 mL of 37% w/v, 13.67 mmol), then diluted with a solution of ethyl acetate/hexanes (1:1) and water. A saturated aqueous solution of sodium bicarbonate was added until the heterogeneous mixture was completely dissolved and the acidic solution was neutralized. The organic layer was removed, and the aqueous layer was further extracted with ethyl acetate/hexanes (1:1, 5×). The combined organic extracts were washed with brine and dried over magnesium sulfate. The solution was filtered, and the filtrate was concentrated in vacuo. The crude residue was purified by flash column chromatography on silica gel (5 to 40% ethyl acetate in hexanes). N-[4-chloro-5-methyl-6-(2-methylphenyl)pyrimidin-2-yl]-1-methyl-1H-pyrazole-4-sulfonamide (0.86 g, 68%) was isolated as a white solid. ESI-MS m/z calc. 377.07132, found 378.26 (M+1)⁺; Retention time: 0.61 minutes; LC method D.

### Step 3: 1-methyl-N-[5-methyl-4-(o-tolyl)-6-phenoxy-pyrimidin-2-yl]pyrazole-4-sulfonamide

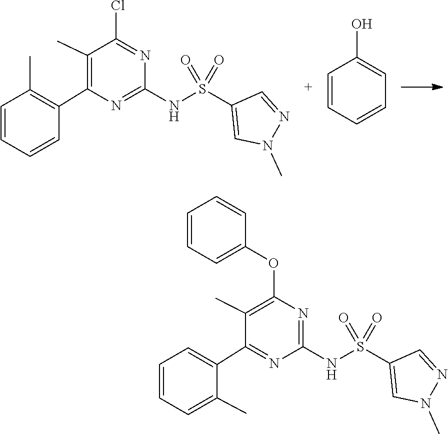

A heterogenous solution of N-[4-chloro-5-methyl-6-(2-methylphenyl)pyrimidin-2-yl]-1-methyl-1H-pyrazole-4-sulfonamide (20 mg, 0.05293 mmol), phenol (approximately 14.94 mg, 14.09 μL, 0.1588 mmol), and cesium carbonate (approximately 86.21 mg, 0.2646 mmol) in NMP (400 μL) was sealed in a vial and the reaction was heated to 110° C. After 16 h the reaction were diluted with DMSO, filtered, and submitted to purification by reverse phase chromatography. 1-methyl-N-[5-methyl-4-(o-tolyl)-6-phenoxy-pyrimidin-2-yl]pyrazole-4-sulfonamide (14.9 mg, 64%). ESI-MS m/z calc. 435.1365, found 436.08 (M+1)⁺; Retention time: 1.54 minutes; LC method A.

### Example 210: Preparation of Compound 562

### Step 1: 1-Methyl-N-[5-methyl-4-(2-methylphenoxy)-6-(o-tolyl)pyrimidin-2-yl]pyrazole-4-sulfonamide

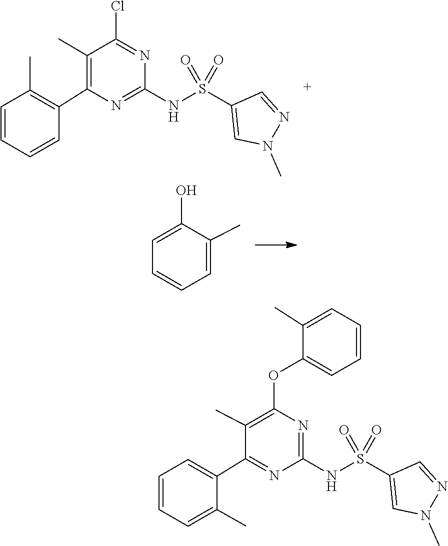

A heterogenous solution of N-[4-chloro-5-methyl-6-(2-methylphenyl)pyrimidin-2-yl]-1-methyl-1H-pyrazole-4-sulfonamide (20 mg, 0.05293 mmol), o-cresol (approximately 17.17 mg, 31.07 μL, 0.1588 mmol), and cesium carbonate (approximately 86.21 mg, 0.2646 mmol) in NMP (400 μL) was sealed in a vial and the reaction was heated to 110° C. and the raction was heated to 150° C. for 16 hours. After 16 hours the reaction was diluted with DMSO, filtered, and purified by reverse phase chromatography to give 1-methyl-N-[5-methyl-4-(2-methylphenoxy)-6-(o-tolyl)pyrimidin-2-yl]pyrazole-4-sulfonamide (32.9 mg, 135%). ESI-MS m/z calc. 449.15216, found 450.09 (M+1)⁺; Retention time: 1.33 minutes; LC method A.

### Example 211: Preparation of Compound 563

### Step 1: N-[4-(3-chlorophenoxy)-5-methyl-6-(o-tolyl)pyrimidin-2-yl]-1-methyl-pyrazole-4-sulfonamide

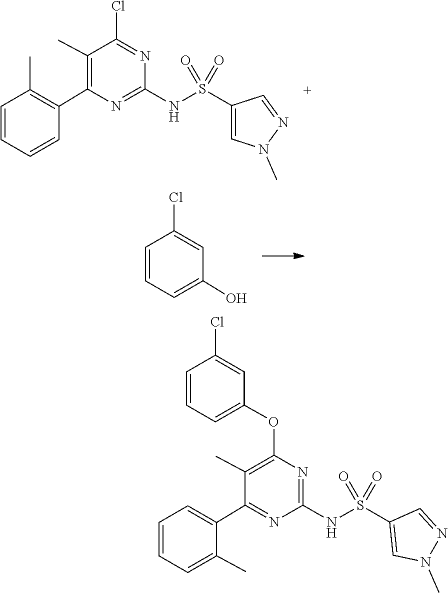

A heterogenous solution of N-[4-chloro-5-methyl-6-(2-methylphenyl)pyrimidin-2-yl]-1-methyl-1H-pyrazole-4-sulfonamide (20 mg, 0.05293 mmol), 3-chlorophenol (approximately 20.41 mg, 0.1588 mmol), and cesium carbonate (approximately 86.21 mg, 0.2646 mmol) in NMP (400 μL) was sealed in a vial and the reaction was heated to 110° C. for 16 hours. The reaction was diluted with DMSO, filtered, and purified by reverse phase chromatography to give N-[4-(3-chlorophenoxy)-5-methyl-6-(o-tolyl)pyrimidin-2-yl]-1-methyl-pyrazole-4-sulfonamide (9.8 mg, 39%). ESI-MS m/z calc. 469.09753, found 470.04 (M+1)⁺; Retention time: 1.71 minutes; LC method A.

### Example 212: Preparation of Compound 564

### Step 1: N-methyl-5-[5-methyl-2-[(1-methylpyrazol-4-yl)sulfonylamino]-6-(o-tolyl)pyrimidin-4-yl]oxy-pyridine-2-carboxamide

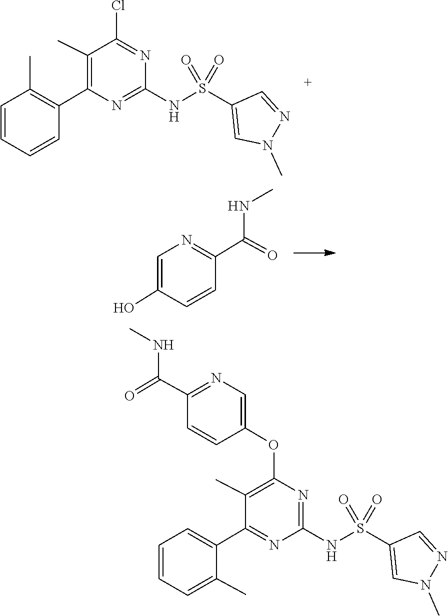

A heterogenous solution of N-[4-chloro-5-methyl-6-(2-methylphenyl)pyrimidin-2-yl]-1-methyl-1H-pyrazole-4-sulfonamide (20 mg, 0.05293 mmol), 5-hydroxy-N-methyl-pyridine-2-carboxamide (approximately 24.16 mg, 0.1588 mmol), and cesium carbonate (approximately 86.21 mg, 0.2646 mmol) in NMP (400 μL) was sealed in a vial and the reaction was heated to 110° C. for 16 hours. The reaction was diluted with DMSO, filtered, and purified by reverse phase chromatography to give N-methyl-5-[5-methyl-2-[(1-methylpyrazol-4-yl)sulfonylamino]-6-(o-tolyl)pyrimidin-4-yl]oxy-pyridine-2-carboxamide (16.9 mg, 65%). ESI-MS m/z calc. 493.15323, found 494.09 (M+1)⁺; Retention time: 1.34 minutes; LC method A.

### Example 213: Preparation of Compound 565

### Step 1: N-methyl-4-[5-methyl-2-[(1-methylpyrazol-4-yl)sulfonylamino]-6-(o-tolyl)pyrimidin-4-yl]oxy-benzamide

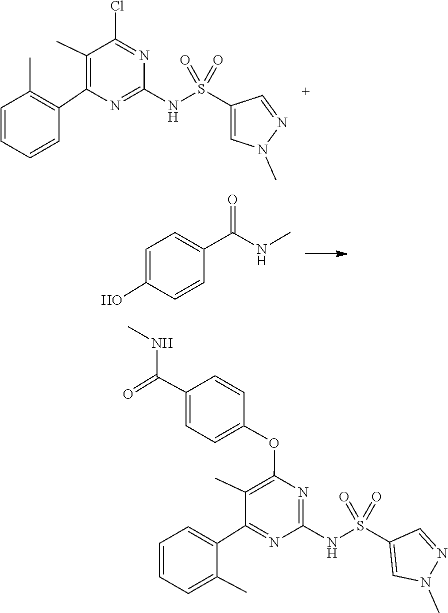

A heterogenous solution of N-[4-chloro-5-methyl-6-(2-methylphenyl)pyrimidin-2-yl]-1-methyl-1H-pyrazole-4-sulfonamide (20 mg, 0.05293 mmol), 4-hydroxy-N-methyl-benzamide (approximately 24.00 mg, 0.1588 mmol), and cesium carbonate (approximately 86.21 mg, 0.2646 mmol) in NMP (400 μL) was sealed in a vial and the reaction was heated to 110° C. for 16 hours. The reaction was diluted with DMSO, filtered, and purified by reverse phase chromatography to give N-methyl-4-[5-methyl-2-[(1-methylpyrazol-4-yl)sulfonylamino]-6-(o-tolyl)pyrimidin-4-yl]oxy-benzamide (15.9 mg, 61%). ESI-MS m/z calc. 492.15796, found 493.1 (M+1)⁺; Retention time: 1.33 minutes; LC method A.

### Example 214: Preparation of Compound 566

### Step 1: N-methyl-3-[5-methyl-2-[(1-methylpyrazol-4-yl)sulfonylamino]-6-(o-tolyl)pyrimidin-4-yl]oxy-benzamide

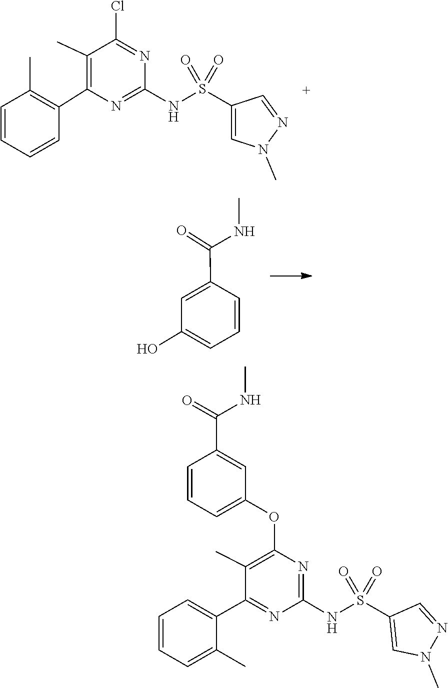

A heterogenous solution of N-[4-chloro-5-methyl-6-(2-methylphenyl)pyrimidin-2-yl]-1-methyl-1H-pyrazole-4-sulfonamide (20 mg, 0.05293 mmol), 3-hydroxy-N-methyl-benzamide (approximately 24.00 mg, 0.1588 mmol), and cesium carbonate (approximately 86.21 mg, 0.2646 mmol) in NMP (400 μL) was sealed in a vial and the reaction was heated to 110° C. for 16 hours. The reaction was diluted with DMSO, filtered, and purified by reverse phase chromatography to give N-methyl-3-[5-methyl-2-[(1-methylpyrazol-4-yl)sulfonylamino]-6-(o-tolyl)pyrimidin-4-yl]oxy-benzamide (16.7 mg, 64%). ESI-MS m/z calc. 492.15796, found 493.1 (M+1)⁺; Retention time: 1.34 minutes; LC method A.

### Example 215: Preparation of Compound 567

### Step 1: 1-Methyl-N-[5-methyl-4-(o-tolyl)-6-(4-piperazin-1-ylphenoxy)pyrimidin-2-yl]pyrazole-4-sulfonamide

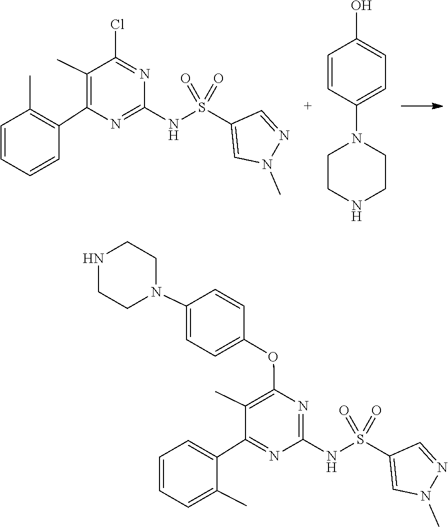 [SMILES: Cc1ccccc1-c1nc(NS(=O)(=O)c2cnn(C)c2)nc(Cl)c1C ; Oc1ccc(N2CCNCC2)cc1 ; Cc1ccccc1-c1nc(NS(=O)(=O)c2cnn(C)c2)nc(Oc2ccc(N3CCNCC3)cc2)c1C]

A heterogenous solution of N-[4-chloro-5-methyl-6-(2-methylphenyl)pyrimidin-2-yl]-1-methyl-1H-pyrazole-4-sulfonamide (20 mg, 0.05293 mmol), 4-piperazin-1-ylphenol (approximately 28.30 mg, 0.1588 mmol), and cesium carbonate (approximately 86.21 mg, 0.2646 mmol) in NMP (400 μL) was sealed in a vial and the reaction was heated to 110° C. for 16 hours. The reaction was diluted with DMSO, filtered, and purified by reverse phase chromatography. 1-Methyl-N-[5-methyl-4-(o-tolyl)-6-(4-piperazin-1-ylphenoxy)pyrimidin-2-yl]pyrazole-4-sulfonamide (7.5 mg, 25%). ESI-MS m/z calc. 519.20526, found 520.2 (M+1)⁺; Retention time: 1.2 minutes; LC method A.

### Example 216: Preparation of Compound 568

### Step 1: N-[4-(cyclohexoxy)-5-methyl-6-(o-tolyl)pyrimidin-2-yl]-1-methyl-pyrazole-4-sulfonamide

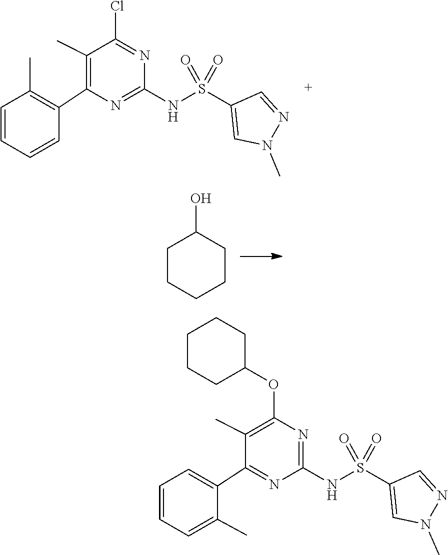

A heterogenous solution of N-[4-chloro-5-methyl-6-(2-methylphenyl)pyrimidin-2-yl]-1-methyl-1H-pyrazole-4-sulfonamide (20 mg, 0.05293 mmol), cyclohexanol (approximately 15.91 mg, 16.54 μL, 0.1588 mmol), and cesium carbonate (approximately 86.21 mg, 0.2646mmol) in NMP (400 μL) was sealed in a vial and the reaction was heated to 150° C. for 16 h, and then was heated for 2 hours at 170° C. The reaction was diluted with DMSO, filtered, and submitted for purification by reverse phase chromatography to give N-[4-(cyclohexoxy)-5 -methyl-6-(o-tolyl)pyrimidin-2-yl]-1-methyl-pyrazole-4-sulfonamide (6.4 mg, 27%). ESI-MS m z calc. 441.18347, found 442.16 (M+1)⁺; Retention time: 1.65 minutes; LC method A.

### Example 217: Preparation of Compound 569

### Step 1: 1-Methyl-N-[5-methyl-4-[3-(4-methylpiperazin-1-yl)phenoxy]-6-(o-tolyl)pyrimidin-2-yl]pyrazole-4-sulfonamide

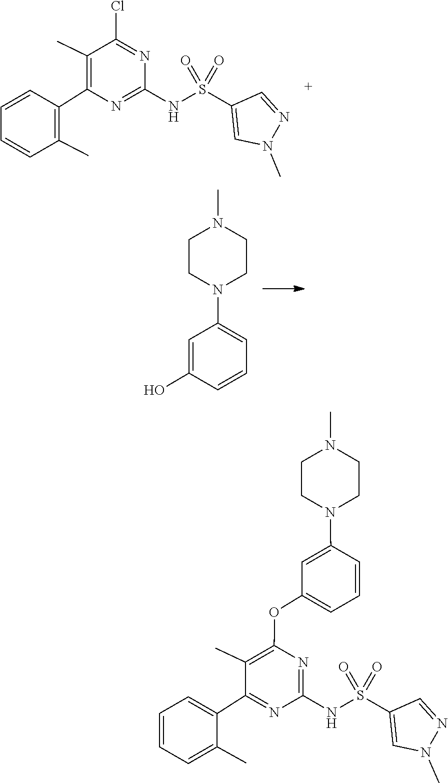 [SMILES: Cc1ccccc1-c1nc(NS(=O)(=O)c2cnn(C)c2)nc(Cl)c1C ; CN1CCN(c2cccc(O)c2)CC1 ; Cc1ccccc1-c1nc(NS(=O)(=O)c2cnn(C)c2)nc(Oc2cccc(N3CCN(C)CC3)c2)c1C]

A heterogeneous solution of N-[4-chloro-5-methyl-6-(2-methylphenyl)pyrimidin-2-yl]-1-methyl-1H-pyrazole-4-sulfonamide (9.446 mg, 0.0250 mmol), 3-(4-methylpiperazin-1-yl)phenol, and potassium carbonate in NMP (50.00 μL) was heated in a sealed vial to 115° C. for 16 hours. The solution was acidified with acetic acid. The sample was purified by reverse phase HPLC (Waters Sunfire C₁₈ column (100×50 mm, 10 m particle size), gradient: 1-99% acetonitrile in water (5 mM HCl) over 15.0 minutes) to afford 1-methyl-N-[5-methyl-4-[3-(4-methylpiperazin-1-yl)phenoxy]-6-(o-tolyl)pyrimidin-2-yl]pyrazole-4-sulfonamide (hydrochloride salt) as a white solid (14.1 mg). ESI-MS m/z calc. 533.2209, found 534.3 (M+1)⁺; Retention time: 1.3 minutes; LC method A.

### Example 218: Preparation of Compound 570

### Step 1: N-[4-[2-chloro-3-(4-methylpiperazin-1-yl)phenoxy]-5-methyl-6-(o-tolyl)pyrimidin-2-yl]-1-methyl-pyrazole-4-sulfonamide

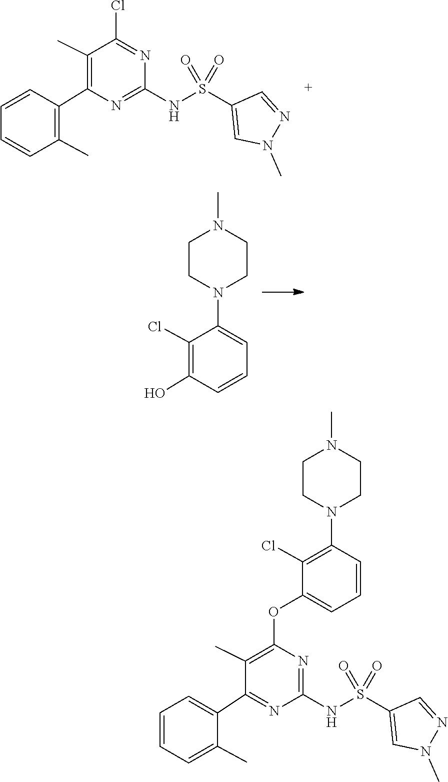 [SMILES: Cc1ccccc1-c1nc(NS(=O)(=O)c2cnn(C)c2)nc(Cl)c1C ; CN1CCN(c2cccc(O)c2Cl)CC1 ; Cc1ccccc1-c1nc(NS(=O)(=O)c2cnn(C)c2)nc(Oc2cccc(N3CCN(C)CC3)c2Cl)c1C]

A heterogeneous solution of N-[4-chloro-5-methyl-6-(2-methylphenyl)pyrimidin-2-yl]-1-methyl-1H-pyrazole-4-sulfonamide (9.446 mg, 0.0250 mmol), 2-chloro-3-(4-methylpiperazin-1-yl)phenol, and potassium carbonate in NMP (50.00 μL) was heated in a sealed vial to 115° C. for 16 hours. The solution was acidified with acetic acid. The sample was purified by reverse phase HPLC (Waters Sunfire C₁₈ column (100×50 mm, 10 m particle size), gradient: 1-99% acetonitrile in water (5 mM HCl) over 15.0 minutes) to afford N-[4-[2-chloro-3-(4-methylpiperazin-1-yl)phenoxy]-5-methyl-6-(o-tolyl)pyrimidin-2-yl]-1-methyl-pyrazole-4-sulfonamide (hydrochloride salt) as a white solid (17 mg). ESI-MS m/z calc. 567.18195, found 568.29 (M+1)⁺; Retention time: 1.36 minutes; LC method A.

### Example 219: Preparation of Compound 571

### Step 1: 1-Methyl-N-[5-methyl-4-[4-(1-methyl-4-piperidyl)phenoxy]-6-(o-tolyl)pyrimidin-2-yl]pyrazole-4-sulfonamide

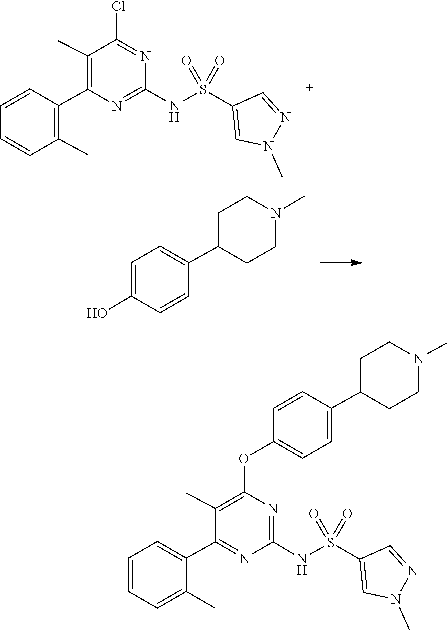

A heterogeneous solution of N-[4-chloro-5-methyl-6-(2-methylphenyl)pyrimidin-2-yl]-1-methyl-1H-pyrazole-4-sulfonamide (9.446 mg, 0.0250 mmol), 4-(1-methyl-4-piperidyl)phenol (approximately 9.563 mg, 0.05000 mmol), and potassium carbonate (approximately 10.37 mg, 0.07500 mmol) in NMP (50.00 μL) was heated in a sealed vial to 115° C. for 16 hours. The solution was acidified with acetic acid. The sample was purified by reverse phase HPLC (Waters Sunfire C₁₈ column (100×50 mm, 10 m particle size), gradient: 1-99% acetonitrile in water (5 mM HCl) over 15.0 minutes) to afford 1-methyl-N-[5-methyl-4-[4-(1 -methyl-4-piperidyl)phenoxy]-6-(o-tolyl)pyrimidin-2-yl]pyrazole-4-sulfonamide (hydrochloride salt) as a white solid (20 mg, 150%). ESI-MS m/z calc. 532.22565, found 533.38 (M+1)⁺; Retention time: 1.3 minutes (LC method A).

### Example 220: Preparation of Compound 572

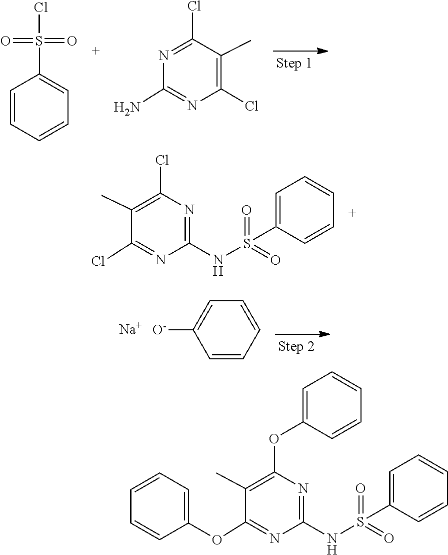

### Step 1: N-(4,6-Dichloro-5-methyl-pyrimidin-2-yl)benzenesulfonamide

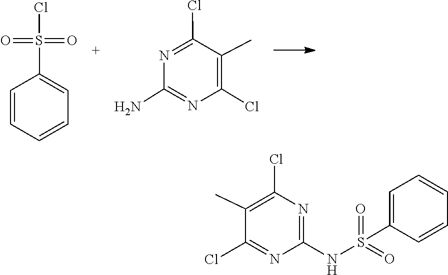

To a solution of 4,6-dichloro-5-methyl-pyrimidin-2-amine (556 mg, 3.123 mmol) in DMA (4 mL) was added NaH (approximately 124.9 mg of 60% w/w, 3.123 mmol). The reaction was stirred at room temperature for 15 minutes. Benzenesulfonyl chloride (399 ptL, 3.127 mmol) was added and the reaction was stirred at room temperature for 21 hours. The reaction was quenched with MeOH and the solvent was evaporated under reduced pressure. EtOAc was added to the reaction and washed with water (×3). The organic layer was dried over Na₂SO₄, filtered and concentrated. The crude product was purified on 80 g of silica gel utilizing a gradient of 0-30% ethyl acetate in hexane to give N-(4,6-dichloro-5-methyl-pyrimidin-2-yl)benzenesulfonamide (240 mg, 24%) as a yellow solid. ESI-MS m/z calc. 316.97925, found 318.1 (M+1)⁺; Retention time: 1.51 minutes; LC method A.

### Step 2: N-(5-Methyl-4,6-diphenoxy-pyrimidin-2-yl)benzenesulfonamide

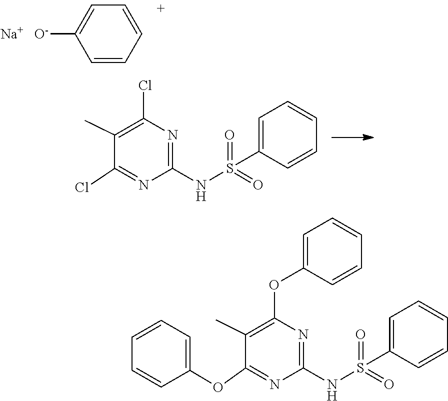 [SMILES: [Na+] ; [O-]c1ccccc1 ; Cc1c(Cl)nc(NS(=O)(=O)c2ccccc2)nc1Cl ; Cc1c(Oc2ccccc2)nc(NS(=O)(=O)c2ccccc2)nc1Oc1ccccc1]

To N-(4,6-dichloro-5-methyl-pyrimidin-2-yl)benzenesulfonamide (86 mg, 0.2703 mmol), sodium phenoxide (31.4 mg, 0.2705 mmol) and DMF (0.8 mL) were added and the reaction was stirred at 110° C. for 7 hours in a pressure vessel. More sodium phenoxide (50 mg) was added and the reaction was heated at 110° C. for 15 hours. The crude product was filtered and purified using a reverse phase HPLC C₁₈ column and a dual gradient run from 1-99% mobile phase B over 30 minutes (Mobile phase A=H₂O (5 mM HCl). Mobile phase B═CH₃CN) to yield N-(5-methyl-4,6-diphenoxy-pyrimidin-2-yl)benzenesulfonamide (32.5 mg, 28%). ¹H NMR (400 MHz, DMSO-d₆) δ 11.33 (s, 1H), 7.56-7.43 (m, 5H), 7.39-7.29 (m, 2H), 7.29-7.22 (m, 2H), 7.21-7.11 (m, 6H), 2.14 (s, 3H). ESI-MS m/z calc. 433.10962, found 434.2 (M+1)⁺; Retention time: 2.06 minutes; LC method A.

### Example 221: Preparation of Compound 573

### Step 1: N-[4-(3-hydroxy-3-methyl-butoxy)-5-methyl-6-(o-tolyl)pyrimidin-2-yl]-1 -methyl-pyrazole-4-sulfonamide

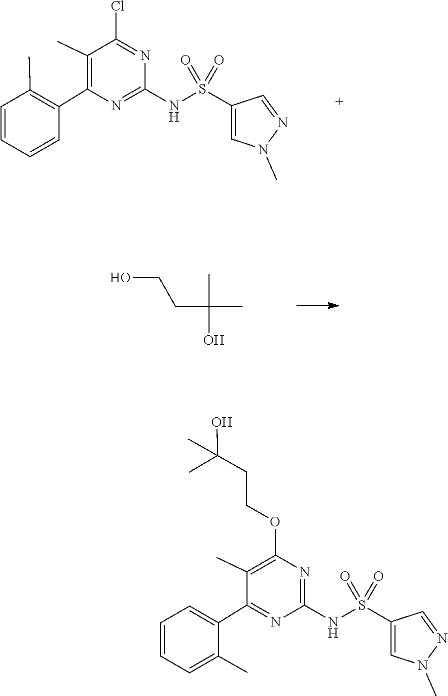

A heterogenous solution of N-[4-chloro-5-methyl-6-(2-methylphenyl)pyrimidin-2-yl]-1-methyl-1H-pyrazole-4-sulfonamide (20 mg, 0.05293 mmol), 3-methylbutane-1,3-diol (approximately 16.54 mg, 0.1588 mmol), and cesium carbonate (approximately 86.21 mg, 0.2646 mmol) in NMP (400 μL) was sealed in a vial and the reaction was heated to 150° C. for 16 hours. It was further heated for 2 h at 170° C. then diluted with DMSO, filtered, and purified by reverse phase chromatography to give N-[4-(3-hydroxy-3-methyl-butoxy)-5-methyl-6-(o-tolyl)pyrimidin-2-yl]-1-methyl-pyrazole-4-sulfonamide (12.6 mg, 53%). ESI-MS m/z calc. 445.17838, found 446.12 (M+1)⁺; Retention time: 1.24 minutes; LC method A.

### Example 222: Preparation of Compound 574

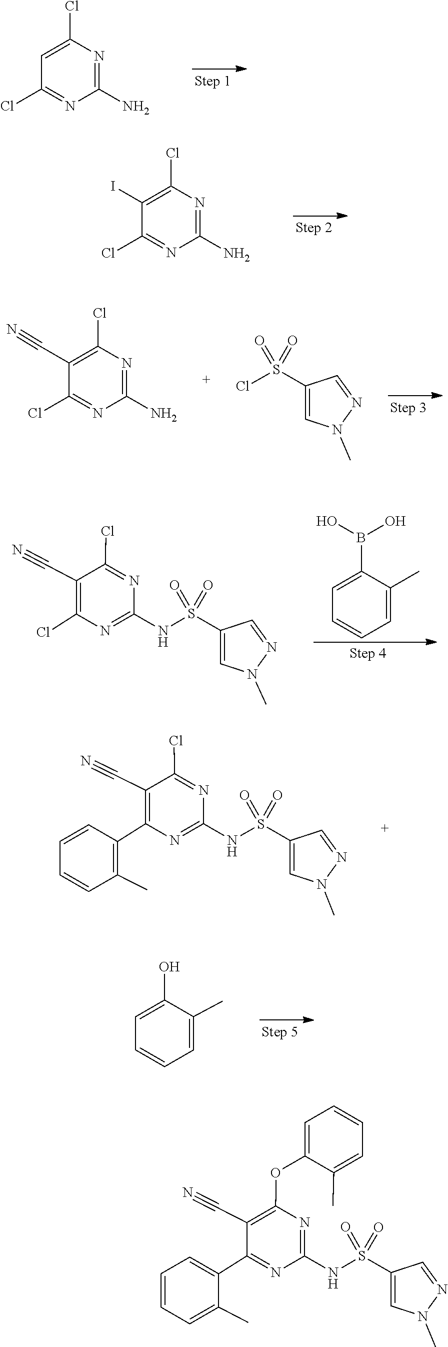

### Step 1: 4,6-Dichloro-5-iodo-pyrimidin-2-amine

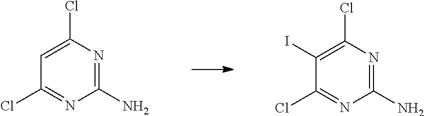 [SMILES: Nc1nc(Cl)cc(Cl)n1 ; Nc1nc(Cl)c(I)c(Cl)n1]

Iodine monochloride (90 g, 554.33 mmol) was dissolved in acetic acid (600 mL) at room temperature and then 4,6-dichloropyrimidin-2-amine (30 g, 182.93 mmol) was added slowly to the solution. The reaction was stirred for 48 hours at room temperature. Precipitate observed and filtered. The solid residue was washed with acetic acid (75 mL) and dried under high vacuum. The acetic acid salt was dissolved in ethyl acetate (300 mL) and added to a stirring saturated sodium bicarbonate aqueous solution (400 mL). The mixture was stirred for 30 minutes then the layers were separated. The aqueous layer was extracted with ethyl acetate (200 mL) and the combined organic layers were dried over sodium sulfate, filtered and concentrated under reduced pressure to give 4,6-dichloro-5-iodo-pyrimidin-2-amine (21 g, 40%) as an off-white solid; ¹H NMR (300 MHz, DMSO-d₆) δ 7.63 (s, 2H). ESI-MS m/z calc. 288.867, found 289.9 (M+1)⁺; Retention time: 1.79 minutes. (LC method K).

### Step 2: 2-Amino-4,6-dichloro-pyrimidine-5-carbonitrile

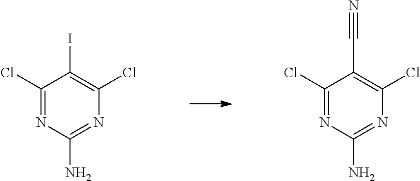 [SMILES: Nc1nc(Cl)c(I)c(Cl)n1 ; N#Cc1c(Cl)nc(N)nc1Cl]

In a 1.0 L round-bottom flask, a solution of 4,6-dichloro-5-iodo-pyrimidin-2-amine (25 g, 86.240 mmol), copper(I) cyanide (31 g, 346.12 mmol) and copper(I) iodide (25 g, 131.27 mmol) in dimethylformamide (375 mL) was bubbled through with nitrogen for 30 minutes. Then, Tetrakis(triphenylphosphine)palladium(0) (10 g, 8.6538 mmol) was added and the reaction mixture was heated at 80° C. overnight. The reaction mixture was cooled to room temperature and the reaction mixture was diluted with water (400 mL) and diethyl ether (300 mL) and the mixture was filtered through Celite and washed with diethyl ether (100 mL). Then, the phases were separated. The combined organic layers were washed with water (100 mL), brine (100 mL), dried over anhydrous sodium sulfate, filtered and concentrated under reduced pressure. The residue was purified on silica gel chromatography, eluting from 0% to 30% ethyl acetate in heptanes to give 2-amino-4,6-dichloro-pyrimidine-5-carbonitrile (7 g, 43%); as an off-white solid. ESI-MS m/z calc. 187.9657, found 188.9 (M+1)⁺; Retention time: 1.58 minutes.

### Step 3: N-(4,6-Dichloro-5-cyano-pyrimidin-2-yl)-1-methyl-pyrazole-4-sulfonamide

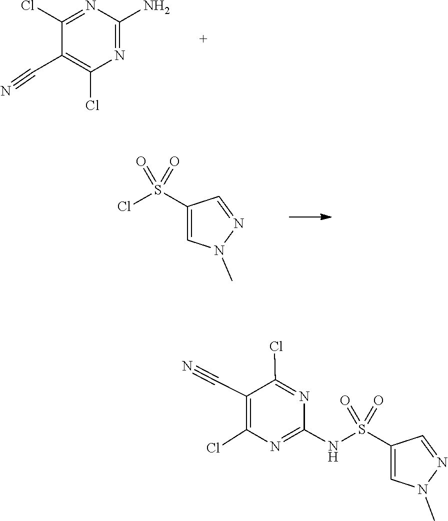

A DMF (9 mL) suspension of NaH (284 mg, 11.83 mmol) was cooled to 0° C. using an ice-water bath and was treated with solid 2-amino-4,6-dichloro-pyrimidine-5-carbonitrile (600 mg, 3.175 mmol). The reaction mixture was stirred for 20 minutes and then treated with solid 1-methylpyrazole-4-sulfonyl chloride (630 mg, 3.488 mmol). The reaction mixture was warmed to room temperature and stirred for 1 hour and then cooled to 0° C. HCl (9 mL of 1 M, 9.000 mmol) was added and the reaction mixture was stirred at 0° C. for 10 minutes upon which the product crystallized out. The reaction mixture was filtered, rinsed with cold water, and dried overnight under vacuum. The crude material was purified on silica using a gradient of ethyl acetate/hexane to give N-(4,6-dichloro-5-cyano-pyrimidin-2-yl)-1-methyl-pyrazole-4-sulfonamide (886 mg, 77%). ESI-MS m/z calc. 331.965, found 333.0 (M+1)⁺; Retention time: 0.99 minutes, (LC method A).

### Step 4: N-[4-chloro-5-cyano-6-(o-tolyl)pyrimidin-2-yl]-1-methyl-pyrazole-4-sulfonamide

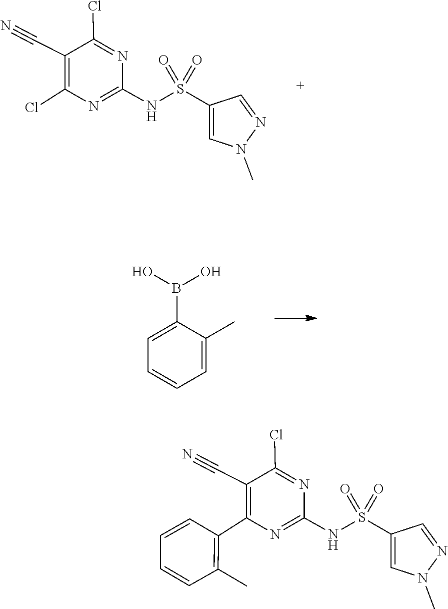

N-(4,6-dichloro-5-cyano-pyrimidin-2-yl)-1-methyl-pyrazole-4-sulfonamide (600 mg, 1.801 mmol), o-tolylboronic acid (265.2 mg, 1.951 mmol), tetrakis(triphenylphosphine)palladium (0) (95 mg, 0.08221 mmol), and 2 M sodium carbonate (2.7 mL of 2 M, 5.400 mmol) in 1,2-dimethoxyethane (6 mL) were combined in a vial. The mixture was purged well with nitrogen, sealed and stirred for 22 hours at 70° C. The reaction mixture was acidified with 1 N HCl, extracted with ethyl acetate. The organic layer was dried over Na₂SO₄, concentrated, and purified on silica using a gradient of ethyl acetate/hexane. Product came out ˜60% ethyl acetate to give N-[4-chloro-5-cyano-6-(o-tolyl)pyrimidin-2-yl]-1 -methyl-pyrazole-4-sulfonamide (125 mg, 17%) ESI-MS m/z calc. 388.05093, found 389.0 (M+1)⁺; Retention time: 1.36 minutes, (LC method A).

### Step 5: N-[5-cyano-4-(2-methylphenoxy)-6-(o-tolyl)pyrimidin-2-yl]-1-methyl-pyrazole-4-sulfonamide

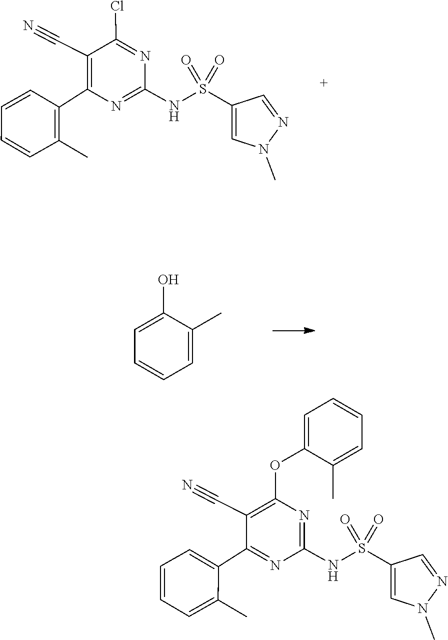

A mixture of N-[4-chloro-5-cyano-6-(o-tolyl)pyrimidin-2-yl]-1-methyl-pyrazole-4-sulfonamide (25 mg, 0.06430 mmol), o-cresol (approximately 9.039 mg, 16.35 μL, 0.08359 mmol) and cesium carbonate (approximately 83.80 mg, 0.2572 mmol) in NMP (1.415 mL) was stirred at 110° C. for 16 hours and then cooled to room temperature. The solution was filtered and purified by reverse phase preparative HPLC using a 15 minutes gradient of 25% MeCN in water to 75% MeCN (HCl modifier) to give N-[5-cyano-4-(2-methylphenoxy)-6-(o-tolyl)pyrimidin-2-yl]-1-methyl-pyrazole-4-sulfonamide (12.6 mg, 43%) ESI-MS m/z calc. 460.13174, found 461.0 (M+1)⁺; Retention time: 1.63 minutes; LC method A. ¹H NMR (400 MHz, DMSO-d₆) δ 12.43 (s, 1H), 7.50 (t, J=2.1 Hz, 1H), 7.49-7.45 (m, 3H), 7.42 (d, J=2.1 Hz, 1H), 7.41-7.38 (m, 2H), 7.36 (ddd, J=7.6, 4.5, 1.7 Hz, 2H), 7.05 (s, 1H), 3.75 (s, 3H), 2.30 (s, 3H), 2.20 (s, 3H).

### Example 223: Preparation of Compound 575

### Step 1: N-[5-cyano-4-[4-(1-methyl-4-piperidyl)phenoxy]-6-(o-tolyl)pyrimidin-2-yl]-1-methyl-pyrazole-4-sulfonamide

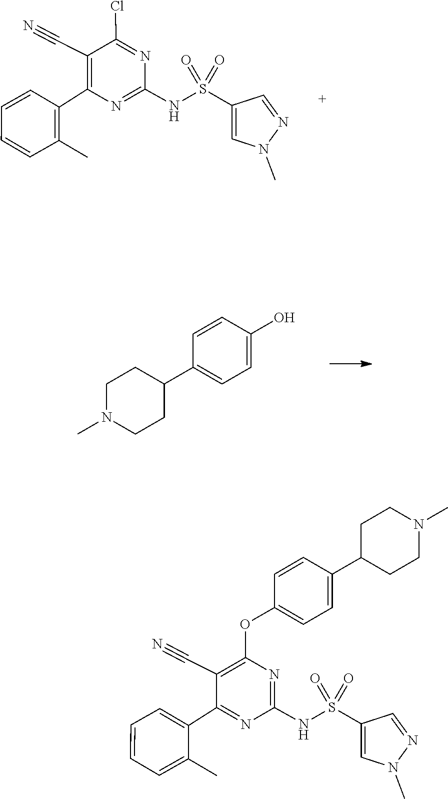 [SMILES: Cc1ccccc1-c1nc(NS(=O)(=O)c2cnn(C)c2)nc(Cl)c1C#N ; CN1CCC(c2ccc(O)cc2)CC1 ; Cc1ccccc1-c1nc(NS(=O)(=O)c2cnn(C)c2)nc(Oc2ccc(C3CCN(C)CC3)cc2)c1C#N]

A mixture of N-[4-chloro-5-cyano-6-(o-tolyl)pyrimidin-2-yl]-1-methyl-pyrazole-4-sulfonamide (25 mg, 0.06430 mmol), 4-(1-methyl-4-piperidyl)phenol (approximately 15.99 mg, 0.08359 mmol) and cesium carbonate (approximately 83.80 mg, 0.2572 mmol) in NMP (1.415 mL) was stirred at 110° C. for 16 hours and then cooled to room temperature. The solution was filtered and purified by reverse phase preparative HPLC using a 15 minute gradient of 25% MeCN in water to 75% MeCN (HCl modifier) to give N-[5-cyano-4-[4-(1-methyl-4-piperidyl)phenoxy]-6-(o-tolyl)pyrimidin-2-yl]-1-methyl-pyrazole-4-sulfonamide (21 mg, 60%). ESI-MS m/z calc. 543.20526, found 544.0 (M+1)⁺; Retention time: 1.17 minutes; LC method A. H NMR (400 MHz, DMSO-d₆) δ 10.54 (s, 1H), 7.57 (s, 1H), 7.48-7.46 (m, 1H), 7.45-7.43 (m, 2H), 7.42-7.39 (m, 3H), 7.39-7.34 (m, 2H), 7.00 (s, 1H), 3.75 (s, 3H), 3.50 (d, J=12.1 Hz, 2H), 3.08 (qt, J=11.6, 4.9 Hz, 2H), 2.95-2.88 (m, 1H), 2.77 (d, J=4.2 Hz, 3H), 2.28 (s, 3H), 2.09-1.97 (m, 4H).

### Example 224: Preparation of Compound 576

Step 1: N-[5-cyano-4-[3-(4-methylpiperazin-1-yl)phenoxy]-6-(o-tolyl)pyrimidin-2-yl]-1-methyl-pyrazole-4-sulfonamide

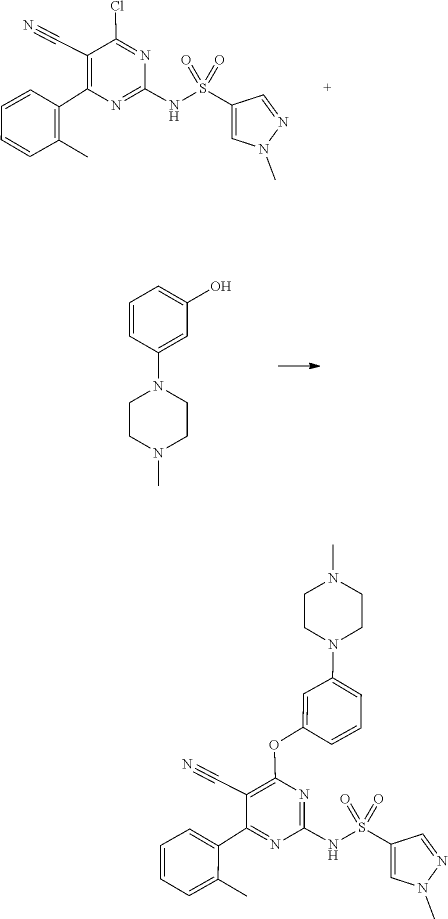 [SMILES: Cc1ccccc1-c1nc(NS(=O)(=O)c2cnn(C)c2)nc(Cl)c1C#N ; CN1CCN(c2cccc(O)c2)CC1 ; Cc1ccccc1-c1nc(NS(=O)(=O)c2cnn(C)c2)nc(Oc2cccc(N3CCN(C)CC3)c2)c1C#N]

A mixture of N-[4-chloro-5-cyano-6-(o-tolyl)pyrimidin-2-yl]-1-methyl-pyrazole-4-sulfonamide (25 mg, 0.06430 mmol), 3-(4-methylpiperazin-1-yl)phenol (approximately 16.74mg, 0.08359 mmol) and cesium carbonate (approximately 83.80 mg, 0.2572 mmol) in NMP (1.415 mL) was stirred at 110° C. for 16 hours and then cooled to room temperature. The solution was filtered and purified by reverse phase preparative HPLC using a 15 minute gradient of 25% MeCN in water to 75% MeCN (HCl modifier) to give N-[5-cyano-4-[3-(4-methylpiperazin-1-yl)phenoxy]-6-(o-tolyl)pyrimidin-2-yl]-1-methyl-pyrazole-4-sulfonamide (24.6 mg, 70%). ESI-MS m/z calc. 544.2005, found 545.0 (M+1)⁺; Retention time: 1.16 minutes; LC method A. ¹H NMR (400 MHz, DMSO-d₆) δ 12.41 (s, 1H), 10.65 (s, 1H), 7.53 (s, 1H), 7.48 (s, 2H), 7.40 (d, J=18.2 Hz, 3H), 7.09 (s, 3H), 6.88 (d, J=7.9 Hz, 1H), 3.96 (s, 2H), 3.75 (s, 3H), 3.51 (s, 2H), 3.16 (d, J=8.0 Hz, 4H), 2.82 (s, 3H), 2.29 (s, 3H).

### Example 225: Preparation of Compound 577

### Step 1: N-[5-cyano-4-[4-(4-methylpiperazin-1-yl)phenoxy]-6-(o-tolyl)pyrimidin-2-yl]-1-methyl-pyrazole-4-sulfonamide

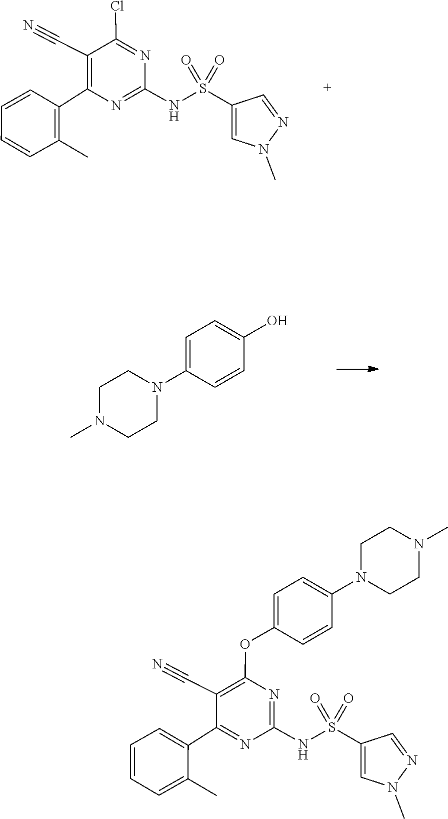 [SMILES: Cc1ccccc1-c1nc(NS(=O)(=O)c2cnn(C)c2)nc(Cl)c1C#N ; CN1CCN(c2ccc(O)cc2)CC1 ; Cc1ccccc1-c1nc(NS(=O)(=O)c2cnn(C)c2)nc(Oc2ccc(N3CCN(C)CC3)cc2)c1C#N]

A mixture of N-[4-chloro-5-cyano-6-(o-tolyl)pyrimidin-2-yl]-1-methyl-pyrazole-4-sulfonamide (25 mg, 0.06430 mmol), 4-(4-methylpiperazin-1-yl)phenol (approximately 16.07 mg, 0.08359 mmol) and cesium carbonate (approximately 83.80 mg, 0.2572 mmol) in NMP (1.415 mL) was stirred at 110° C. for 16 hours and then cooled to room temperature. The solution was filtered and purified by reverse phase preparative HPLC using a 15 minute gradient of 25% MeCN in water to 75% MeCN (HCl modifier) to give N-[5-cyano-4-[4-(4-methylpiperazin-1-yl)phenoxy]-6-(o-tolyl)pyrimidin-2-yl]-1-methyl-pyrazole-4-sulfonamide (8.4 mg, 24%). ESI-MS m/z calc. 544.2005, found 545.0 (M+1)⁺; Retention time: 1.13 minutes; LC method A. ¹H NMR (400 MHz, DMSO-d₆) δ 12.37 (s, 1H), 10.25 (s, 1H), 7.60 (s, 1H), 7.46 (d, J=7.4 Hz, 1H), 7.42-7.35 (m, 3H), 7.31 (d, J=9.0 Hz, 2H), 7.19 (d, J=9.1 Hz, 2H), 7.13 (s, 1H), 3.91 (s, 3H), 3.75 (s, 3H), 2.84 (s, 6H), 2.69 (s, 3H), 2.32 (s, 1H), 2.28 (s, 3H), 2.18 (s, 1H), 1.91 (d, J=7.4 Hz, 1H).

### Example 226: Preparation of Compound 578

### Step 1: N-[4-[2-chloro-3-(4-methylpiperazin-1-yl)phenoxy]-5-cyano-6-(o-tolyl)pyrimidin-2-yl]-1-methyl-pyrazole-4-sulfonamide

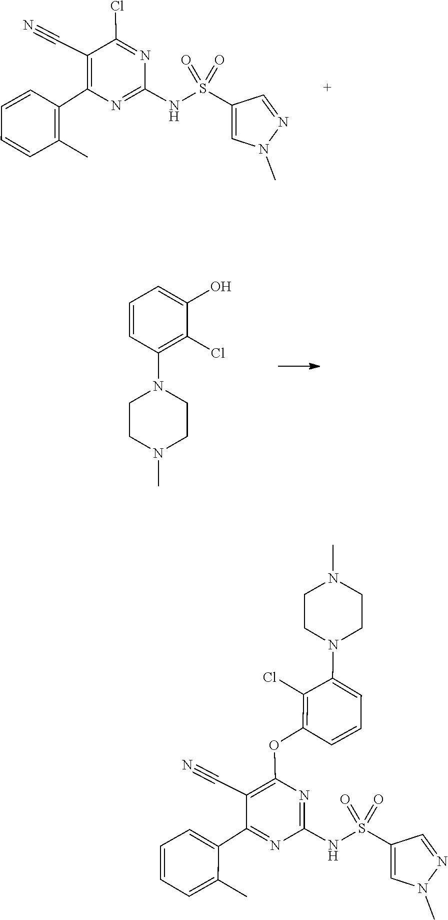 [SMILES: Cc1ccccc1-c1nc(NS(=O)(=O)c2cnn(C)c2)nc(Cl)c1C#N ; CN1CCN(c2cccc(O)c2Cl)CC1 ; Cc1ccccc1-c1nc(NS(=O)(=O)c2cnn(C)c2)nc(Oc2cccc(N3CCN(C)CC3)c2Cl)c1C#N]

A mixture of N-[4-chloro-5-cyano-6-(o-tolyl)pyrimidin-2-yl]-1-methyl-pyrazole-4-sulfonamide (25 mg, 0.06430 mmol), 2-chloro-3-(4-methylpiperazin-1-yl)phenol (approximately 18.95 mg, 0.08359 mmol) and cesium carbonate (approximately 83.80 mg, 0.2572 mmol) in NMP (1.415 mL) was stirred at 110° C. for 16 hours and then cooled to room temperature. The solution was filtered and purified by reverse phase preparative HPLC using a 15 minute gradient of 25% MeCN in water to 75% MeCN (HCl modifier) to give N-[4-[2-chloro-3-(4-methylpiperazin-1-yl)phenoxy]-5-cyano-6-(o-tolyl)pyrimidin-2-yl]-1-methyl-pyrazole-4-sulfonamide (16.2 mg, 43%). ESI-MS m/z calc. 578.16156, found 579.0 (M+1)⁺; Retention time: 1.2 minutes; LC method A. ¹H NMR (400 MHz, DMSO-d₆) δ 12.56 (s, 1H), 10.87 (s, 1H), 7.67 (s, 1H), 7.57 (t, J=8.1 Hz, 1H), 7.45 (dt, J=16.8, 7.8 Hz, 4H), 7.37-7.30 (m, 2H), 7.01 (s, 1H), 3.79 (s, 3H), 3.55 (s, 4H), 3.22 (s, 4H), 2.85 (s, 3H), 2.29 (s, 3H).

### Example 227: Preparation of Compound 579

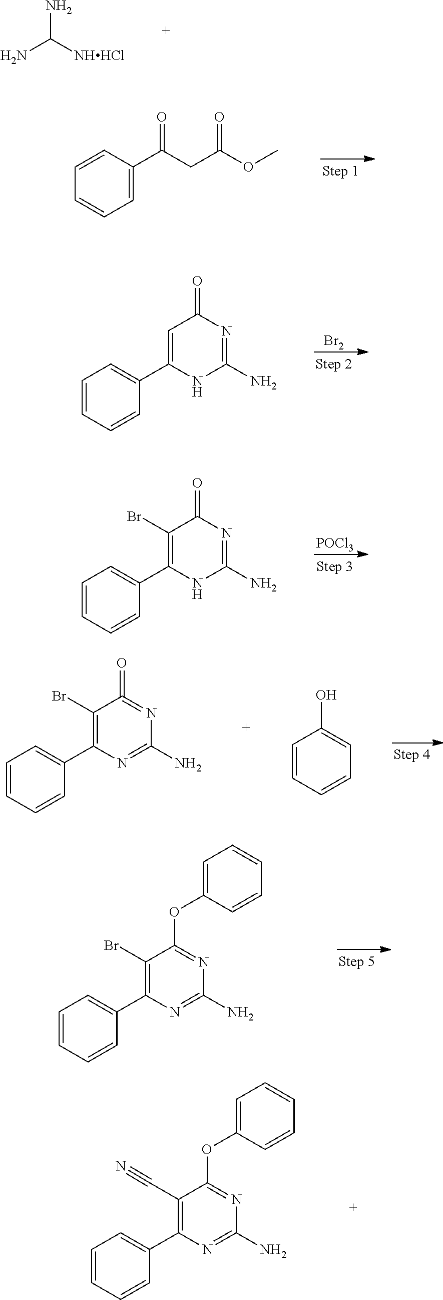

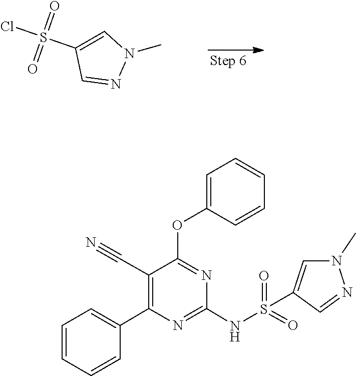

### Step 1: 2-Amino-6-phenyl-1H-pyrimidin-4-one

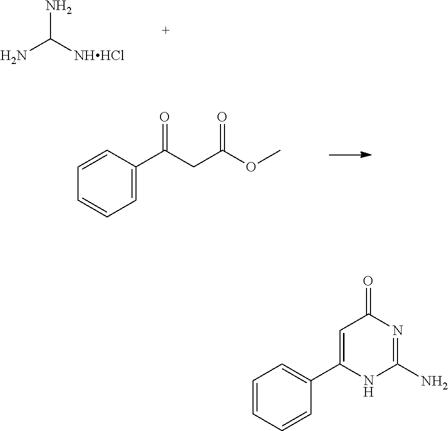

To a solution of methyl 3-oxo-3-phenyl-propanoate (25 g, 140.3 mmol) and guanidine (hydrochloride salt) (16.28 g, 170.4 mmol) in methanol (450 mL) was added potassium tert-butoxide (79.17 g, 705.5 mmol) in portions over 45 minutes at room temperature with vigorous stirring. The reaction mixture was stirred at rt overnight. The reaction was heated at 60° C. for 72 hours. The reaction mixture was filtered, and the filtrate was concentrated (˜⅔ of the volume was removed). The slurry was acidified to pH ˜5 with 6 N HCl (˜100 mL). The precipitate was filtered, washed with water, hexane and dried in a vacuum oven to yield 2-amino-6-phenyl-1H-pyrimidin-4-one as a cream solid. ¹H NMR (400 MHz, DMSO-d₆) δ 10.84 (s, 1H), 7.98-7.89(m, 2H), 7.48-7.39 (m, 3H), 6.63 (s, 2H), 6.10 (s, 1H). ESI-MS m/z calc. 187.07455, found 188.1 (M+1)⁺; Retention time: 0.27 minutes (LC method D).

### Step 2: 2-Amino-5-bromo-6-phenyl-pyrimidin-4-ol

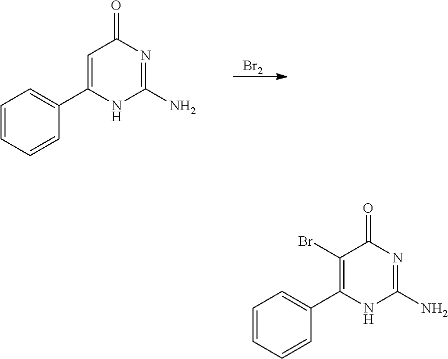 [SMILES: Nc1nc(=O)cc(-c2ccccc2)[nH]1 ; "Br2" ; Nc1nc(=O)c(Br)c(-c2ccccc2)[nH]1]

To 2-amino-6-phenyl-pyrimidin-4-ol (14.51 g, 77.51 mmol) was added glacial acetic acid (418.0 mL). The mixture was heated to 80° C. and bromine (approximately 12.39 g, 3.994 mL, 77.51 mmol) was added dropwise. The clear solution was allowed to stir at 80° C. for 35 minutes and it was cooled to room temperature. A precipitate was observed. The mixture was evaporated to dryness under vacuum at 50° C. and the resulting solid was heated at reflux with water (400 mL) for 30 minutes. The reaction mixture was cooled and filtered, and the solid was washed very well with cold water. The solid was dried in a vacuum oven to yield 2-amino-5-bromo-6-phenyl-pyrimidin-4-ol (20.6 g, 100%) as a cream solid. ¹H NMR (400 MHz, DMSO-d₆) δ 7.58-7.50 (m, 2H), 7.49-7.39 (m, 3H), 6.91 (s, 2H). ESI-MS m/z calc. 264.98508, found 266.0 (M+1)⁺; Retention time: 0.35 minutes (LC method D).

### Step 3: 5-Bromo-4-chloro-6-phenyl-pyrimidin-2-amine

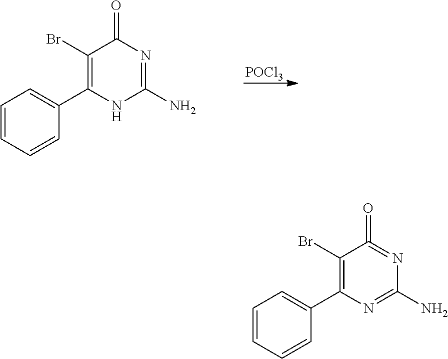

To 2-amino-5-bromo-6-phenyl-pyrimidin-4-ol (approximately 5.000 g, 18.79 mmol) in toluene (108 mL) was added phosphorus oxychloride (21 mL, 225.3 mmol). The resulting reaction was heated at 120° C. for 18 hours. The solvent was evaporated under reduced pressure. Ice and EtOAc were added to the reaction. The two layers were separated, and the aqueous layer was extracted with EtOAc (×2). The organic layer was dried over sodium sulfate, filtered, and the solvent was removed in vacuo. The crude product was purified by flash chromatography on 220 g of silica gel eluting with a gradient of 0-30% ethyl acetate in hexane, followed by 10% MeOH in DCM. The product was purified a second time on 120 g of silica gel utilizing a gradient of 0-100% ethyl acetate in DCM to yield 5-bromo-4-chloro-6-phenyl-pyrimidin-2-amine (362.9 mg, 7%). ESI-MS m/z calc. 282.9512, found 283.9 (M+1)⁺; Retention time: 1.46 minutes (LC method A).

### Step 4: 5-Bromo-4-phenoxy-6-phenyl-pyrimidin-2-amine

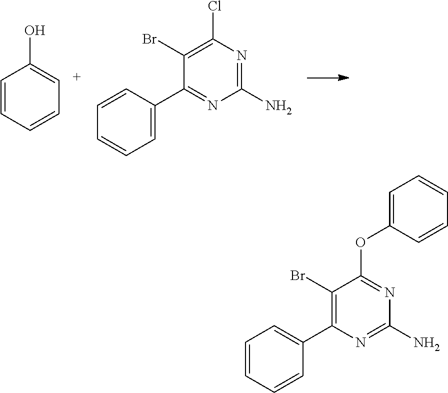

To 5-bromo-4-chloro-6-phenyl-pyrimidin-2-amine (1 g, 3.514 mmol) and potassium carbonate (1.46 g, 10.56 mmol) was added phenol (500 mg, 5.313 mmol) followed by DMF (8.5 mL). The mixture was heated at 110° C. for 2 hours. EtOAc and water were added to the reaction. The two layers were separated after shaking. The aqueous layer was extracted with EtOAc (×1). The combined organic layer was washed with water (×3). The organic layer was dried over Na₂SO₄, filtered and the solvent was evaporated under reduced pressure. The solid was washed with water and hexane and dried in a vacuum oven to yield 5-bromo-4-phenoxy-6-phenyl-pyrimidin-2-amine (902.1 mg, 75%) as a cream solid. ¹H NMR (400 MHz, DMSO-d₆) δ 7.70-7.57 (m, 2H), 7.54-7.41 (m, 5H), 7.37-7.15 (m, 3H), 6.87 (s, 2H). ESI-MS m/z calc. 341.0164, found 342.0 (M+1)⁺; Retention time: 1.61 minutes; LC method A.

### Step 5: 2-Amino-4-phenoxy-6-phenyl-pyrimidine-5-carbonitrile

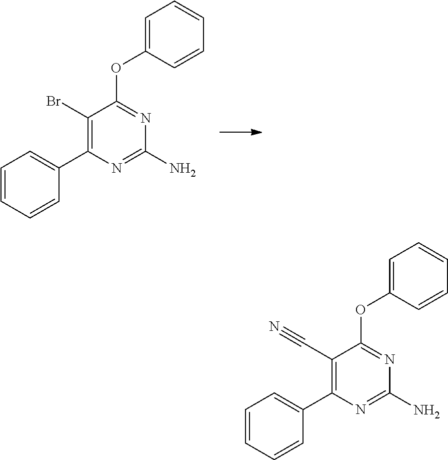 [SMILES: Nc1nc(Oc2ccccc2)c(Br)c(-c2ccccc2)n1 ; N#Cc1c(Oc2ccccc2)nc(N)nc1-c1ccccc1]

5-Bromo-4-phenoxy-6-phenyl-pyrimidin-2-amine (400 mg, 1.169 mmol) and cyanocopper (128.0 mg, 1.429 mmol) in DMF (7 mL) was heated up to 150° C. for 8 hours in a pressure vessel. To the mixture was added to water and extracted with EtOAc (×7). The combined organic phase was dried over anhydrous sodium sulfate, filtered and concentrated under reduced pressure. The crude product was purified on 80 g of silica gel utilizing a gradient of 0-50% ethyl acetate in hexane to yield 2-amino-4-phenoxy-6-phenyl-pyrimidine-5-carbonitrile (169 mg, 50%). ESI-MS m/z calc. 288.1011, found 289.2 (M+1)⁺; Retention time: 1.4 minutes (LC method A).

### Step 6: N-(5-cyano-4-phenoxy-6-phenyl-pyrimidin-2-yl)-1-methyl-pyrazole-4-sulfonamide

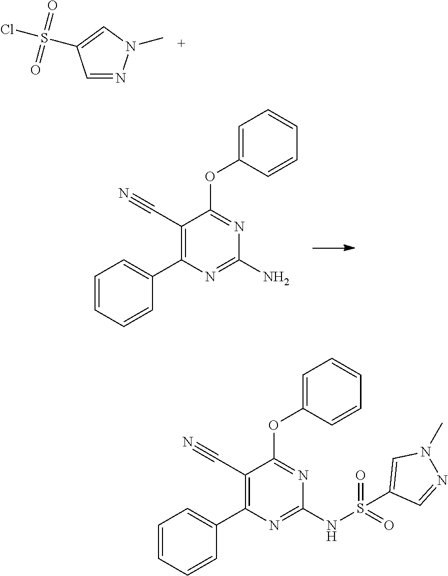

To 2-amino-4-phenoxy-6-phenyl-pyrimidine-5-carbonitrile (150 mg, 0.5203 mmol), 1-methylpyrazole-4-sulfonyl chloride (380 mg, 2.104 mmol) and pyridine (8.1 mL) were added and the reaction was heated in the microwave instrument at 150° C. for 12.5 hours. Water and EtOAc were added to the reaction and the two layers were separated. The organic layer was washed with water (×2), dried over Na₂SO₄, filtered and the solvent was evaporated under reduced pressure. The crude product was purified on 24 g of silica gel utilizing a gradient of 0-100% ethyl acetate in hexane to yield N-(5-cyano-4-phenoxy-6-phenyl-pyrimidin-2-yl)-1 -methyl-pyrazole-4-sulfonamide (149.9 mg, 67%) as a yellow solid. ¹H NMR (400 MHz, DMSO-d₆) δ 12.40 (s, 1H), 8.03-7.77 (m, 2H), 7.70-7.51 (m, 6H), 7.50-7.33 (m, 3H), 7.23 (s, 1H), 3.75 (s, 3H). ESI-MS m/z calc. 432.10046, found 433.1 (M+1)⁺; Retention time: 1.59 minutes; LC method A.

### Example 228: Preparation of Compound 580

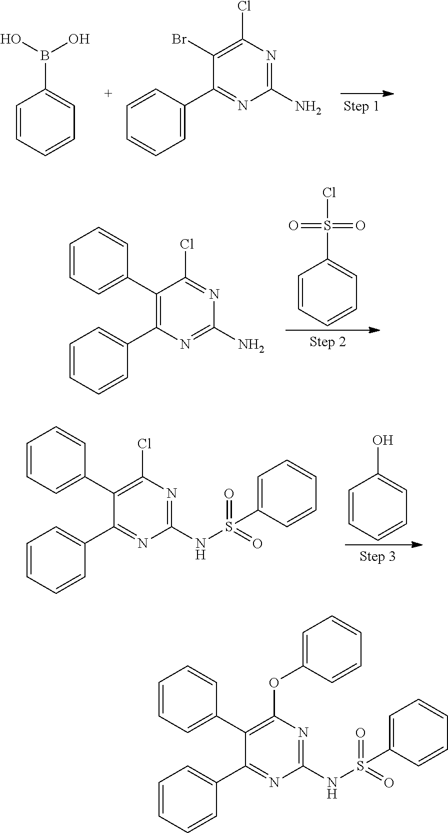

### Step 1: 4-Chloro-5,6-diphenyl-pyrimidin-2-amine

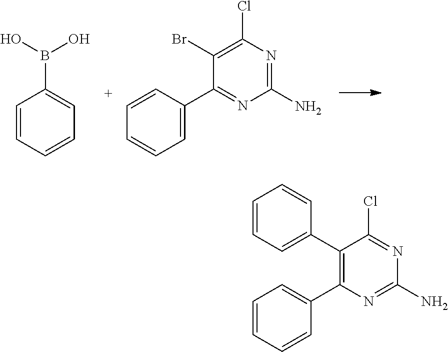 [SMILES: OB(O)c1ccccc1 ; Nc1nc(Cl)c(Br)c(-c2ccccc2)n1 ; Nc1nc(Cl)c(-c2ccccc2)c(-c2ccccc2)n1]

A mixture of 5-bromo-4-chloro-6-phenyl-pyrimidin-2-amine (500 mg, 1.757 mmol), phenylboronic acid (247.7 mg, 2.031 mmol), Pd(dppf)Cl₂ (130.6 mg, 0.1785 mmol), and potassium carbonate (2 mL of 2 M, 4.000 mmol) in 1,2-dimethoxyethane (3.8 mL) was degassed with nitrogen and the reaction was stirred at 130° C. for 15 minutes. The reaction was filtered. EtOAc and water were added, and the two layers were separated. The organic layer was dried over Na₂SO₄, filtered and concentrated. The crude product was purified on 24 g of silica gel utilizing a gradient of 0-50% ethyl acetate in hexane to yield 4-chloro-5,6-diphenyl-pyrimidin-2-amine (152.9 mg, 31%) as a white solid. ESI-MS m/z calc. 281.07196, found 282.1 (M+1)⁺; Retention time: 1.55 minutes (LC method A).

### Step 2: N-(4-Chloro-5,6-diphenyl-pyrimidin-2-yl)benzenesulfonamide

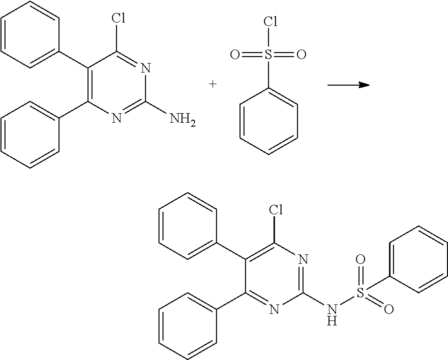 [SMILES: Nc1nc(Cl)c(-c2ccccc2)c(-c2ccccc2)n1 ; O=S(=O)(Cl)c1ccccc1 ; O=S(=O)(Nc1nc(Cl)c(-c2ccccc2)c(-c2ccccc2)n1)c1ccccc1]

To a solution of 4-chloro-5,6-diphenyl-pyrimidin-2-amine (150 mg, 0.5324 mmol) in N,N-dimethylacetamide (2.5 mL) at 0° C. was added NaH (31 mg of 60% w/w, 0.7751 mmol). The reaction was removed from the ice bath and stirred at room temperature for 10 minutes. The reaction was cooled to 0° C. and a solution of benzenesulfonyl chloride (approximately 210.4 mg, 152.0 μL, 1.191 mmol) in N,N-dimethylacetamide (500 μL) was added dropwise and the reaction was allowed to warm to room temperature and stirred for 6 hours. The reaction was quenched with MeOH and the solvent was evaporated under reduced pressure. EtOAc was added to the reaction and it was washed with water (×3). The organic layer was dried over Na₂SO₄, filtered and concentrated. The crude product was purified on 24 g of silica gel utilizing a gradient of 0-30% ethyl acetate in DCM to yield N-(4-chloro-5,6-diphenyl-pyrimidin-2-yl)benzenesulfonamide (53.2 mg, 24%) as a white solid. ESI-MS m/z calc. 421.0652, found 422.1 (M+1)⁺; Retention time: 1.86 minutes (LC method A).

### Step 3: N-(4-Phenoxy-5,6-diphenyl-pyrimidin-2-yl)benzenesulfonamide

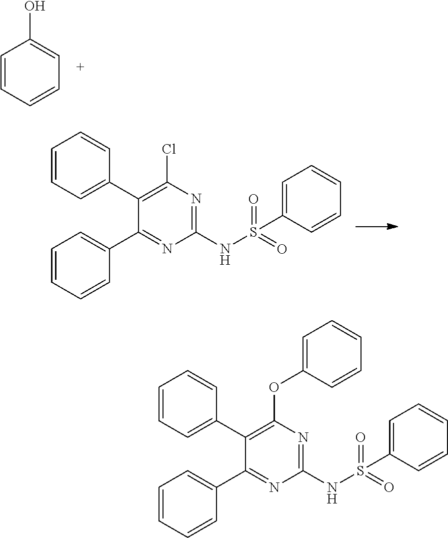 [SMILES: Oc1ccccc1 ; O=S(=O)(Nc1nc(Cl)c(-c2ccccc2)c(-c2ccccc2)n1)c1ccccc1 ; O=S(=O)(Nc1nc(Oc2ccccc2)c(-c2ccccc2)c(-c2ccccc2)n1)c1ccccc1]

To N-(4-chloro-5,6-diphenyl-pyrimidin-2-yl)benzenesulfonamide (24 mg, 0.05689 mmol) and potassium carbonate (24.8 mg, 0.1794 mmol) was added phenol (10 mg, 0.1063mmol) followed by N,N-dimethylformamide (500 μL). The mixture was heated at 110° C. for 110 hours. The reaction was allowed to cool down to room temperature, filtered and purified using a reverse phase HPLC₁₈ column and a gradient of 1-99% over 15 minutes of acetonitrile in water containing 5 mM HCl, to yield N-(4-phenoxy-5,6-diphenyl-pyrimidin-2-yl)benzenesulfonamide (4.7 mg, 17%) as a white solid. ESI-MS m/z calc. 479.13037, found 480.1 (M+1)⁺; Retention time: 2.01 minutes (LC method A).

### Example 229: Preparation of Compound 581

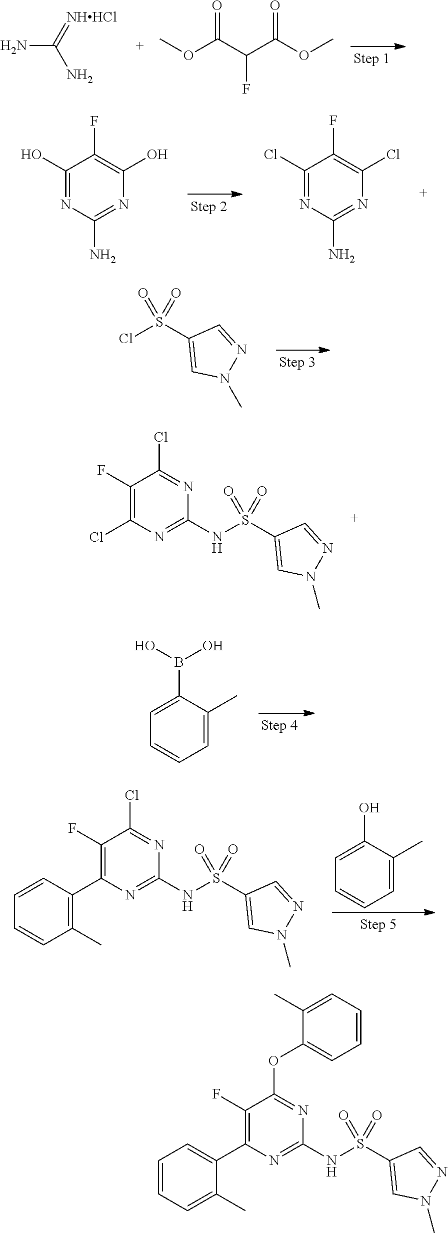

### Step 1: 2-Amino-5-fluoro-pyrimidine-4,6-diol

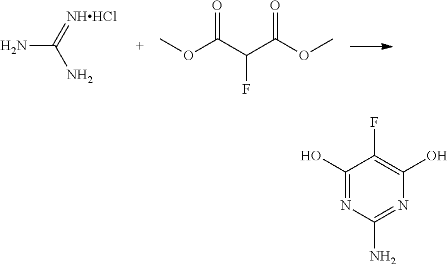

An oil suspension of sodium hydride (65.55 g, 60% w/w, 1.6389 mol) was added gradually to ethanol (1600 mL) in 30 minutes. At 30° C., guanidine (hydrochloride salt) (55.5 g, 580.96 mmol) and dimethyl 2-fluoropropanedioate (80.2 g, 534.29 mmol) were successively added and the reaction mixture was stirred at 70° C. overnight. Once cooled to room temperature, the crude mixture was concentrated under reduced pressure to remove most of the ethanol. Water (1000 mL) was added until dissolution, the resulting solution was washed twice with methyl tertbutylether (2×100 mL). The aqueous layer was partially concentrated under vacuum (100 mL removed) and acidified to pH of 4-5 using acetic acid (40 mL) at 35-40° C. The mixture was cooled down to 8° C., the solids were collected by filtration and washed with water (100 mL). The solid was dried under vacuum at 60° C. for 24 hours to give 2-amino-5-fluoro-pyrimidine-4,6-diol hydrate (76.23 g, 87%) as a pink solid.

### Step 2: 4,6-Dichloro-5-fluoro-pyrimidin-2-amine

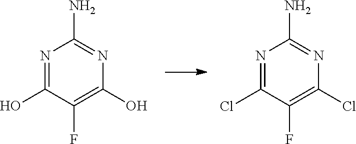 [SMILES: Nc1nc(O)c(F)c(O)n1 ; Nc1nc(Cl)c(F)c(Cl)n1]

2-Amino-5-fluoro-pyrimidine-4,6-diol hydrate (74 g, 453.69 mmol) was added slowly to phosphorus oxychloride (345.45 g, 210 mL, 2.2530 mol). The reaction was then heated for 3 hours at 105° C. The solution was poured in water (700 mL) in 30 minutes and neutralized to pH ˜5 with a 50% aqueous sodium hydroxide solution (900 mL) and heated to 80° C. for 1 hour. The solution was cooled to 20° C. and the suspension was collected by filtration, washed with water and dried under vacuum at 60° C. for 24 hours. The solid was triturated in dichloromethane (˜500 mL), filtered and dried. The resulting solid was dissolved in ethyl acetate (800 mL). Undissolved solids were filtered off using Celite, washed with ethyl acetate (200 mL) and discarded. The filtrate was concentrated, and the residue was dissolved in isopropyl alcohol (600 mL) at 82° C. Water (200 mL) was added dropwise until appearance of a suspension. The mixture was allowed to cool down and the crystallization occurred at 64° C. After 1 hour, the solid was collected by filtration at 20° C., washed with a mixture of isopropyl alcohol and water (1:1, 100 mL) and dried under vacuum providing compound 4,6-dichloro-5-fluoro-pyrimidin-2-amine (38 g, 43%) as a beige solid.

### Step 3: N-(4,6-Dichloro-5-fluoro-pyrimidin-2-yl)-1-methyl-pyrazole-4-sulfonamide

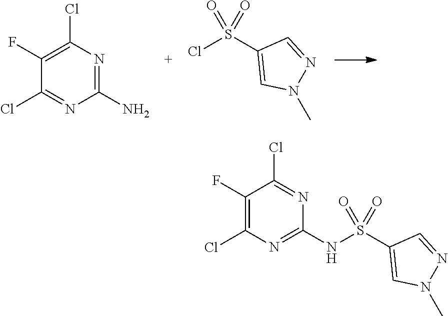

A DMF (45 mL) suspension of NaH (1.4 g, 58.34 mmol) was cooled to 0° C. using an ice-water bath and was treated with solid 4,6-dichloro-5-fluoro-pyrimidin-2-amine (3 g, 16.49 mmol). The reaction mixture was stirred for 20 minutes and then treated with solid 1-methylpyrazole-4-sulfonyl chloride (3.3 g, 18.27 mmol). The reaction mixture was warmed to room temperature and stirred for 1 hour and then cooled to 0° C. Aqueous HCl (50 mL of 1 M, 50.00 mmol) was added and the reaction mixture was stirred at 0° C. for 10 minutes upon which the product crystallized out. The reaction mixture was filtered, rinsed with cold water, and dried overnight under vacuum suction followed by high vacuum to give N-(4,6-dichloro-5-fluoro-pyrimidin-2-yl)-1-methyl-pyrazole-4-sulfonamide (4.4103 g, 79%). ¹H NMR (400 MHz, DMSO-d₆) δ 12.33 (s, 1H), 8.41 (s, 1H), 7.89 (s, 1H), 3.88 (s, 3H). ESI-MS m/z calc. 324.96033, found 326.0 (M+1)⁺; Retention time: 1.11 minutes (LC method A).

### Step 4: N-[4-chloro-5-fluoro-6-(o-tolyl)pyrimidin-2-yl]-1-methyl-pyrazole-4-sulfonamide

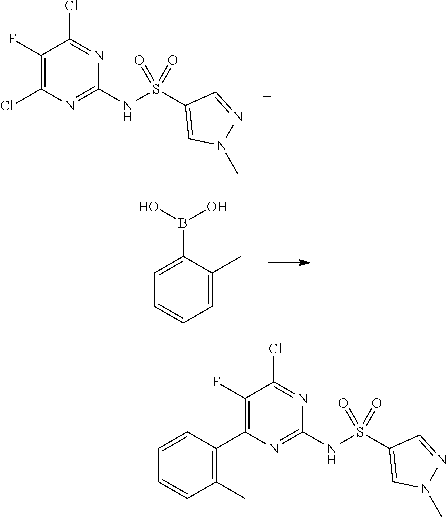

N-(4,6-dichloro-5-fluoro-pyrimidin-2-yl)-1-methyl-pyrazole-4-sulfonamide (0.8 g, 2.453 mmol), o-tolylboronic acid (350 mg, 2.574 mmol), tetrakis(triphenylphosphine)palladium (0) (140 mg, 0.1212 mmol), and 2 M sodium carbonate (4 mL of 2 M, 8.000 mmol) in 1,2-dimethoxyethane (8 mL) were combined in a vial. The mixture was purged well with nitrogen, sealed and stirred for 15 hours at 75° C. The reaction mixture was diluted with water, extracted with ethyl acetate. The organic layer was dried over Na₂SO₄, concentrated, and purified on silica using a gradient of ethyl acetate/hexane. Product came out ˜50% ethyl acetate. The product from silica column was purified on reverse phase HPLC (HCl modifier, 25-75% ACN-H₂O) to give N-[4-chloro-5-fluoro-6-(o-tolyl)pyrimidin-2-yl]-1-methyl-pyrazole-4-sulfonamide (443 mg, 47%)¹H NMR (400 MHz, DMSO-d₆) δ 8.31 (s, 1H), 7.79 (s, 1H), 7.49-7.43 (m, 1H), 7.41-7.32 (m, 3H), 3.84 (s, 3H), 2.22 (s, 3H). ESI-MS m/z calc. 381.04626, found 382.0 (M+1)⁺; Retention time: 1.49 minutes; LC method A.

### Step 5: N-[5-fluoro-4-(2-methylphenoxy)-6-(o-tolyl)pyrimidin-2-yl]-1-methyl-pyrazole-4-sulfonamide

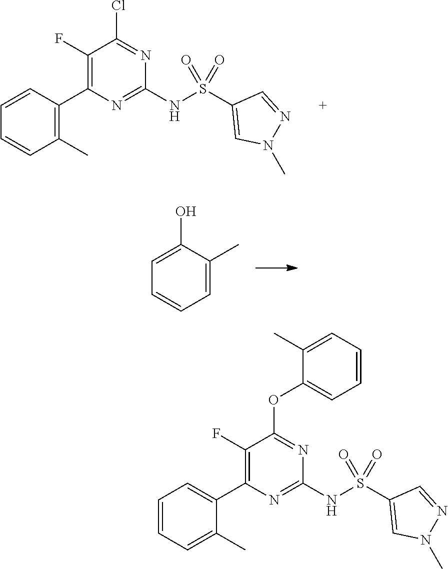

A mixture of o-cresol (approximately 18.42 mg, 33.33 μL, 0.1703 mmol), N-[4-chloro-5-fluoro-6-(o-tolyl)pyrimidin-2-yl]-1-methyl-pyrazole-4-sulfonamide (50 mg, 0.1310 mmol), and cesium carbonate (approximately 170.7 mg, 0.5240 mmol) in NMP (2.830 mL) was stirred at 110° C. for 22 hours and then cooled to room temperature. The solution was filtered and the resulting residue diluted with 0.8 mL MeOH, and purified by reverse phase chromatography using a 15 min gradient of 25% MeCN in water to 75% MeCN (HCl modifier) to give N-[5-fluoro-4-(2-methylphenoxy)-6-(o-tolyl)pyrimidin-2-yl]-1-methyl-pyrazole-4-sulfonamide (14.9 mg, 25%). ESI-MS m/z calc. 453.12708, found 454.0 (M+1)⁺; Retention time: 1.79 minutes; LC method A. ¹H NMR (400 MHz, DMSO-d₆) δ 11.62 (s, 1H), 7.47-7.44 (m, 2H), 7.43-7.41 (m, 1H), 7.39 (d, J=1.9 Hz, 2H), 7.37 (d, J=1.8 Hz, 1H), 7.36-7.35 (m, 1H), 7.35-7.33 (m, 1H), 7.33-7.28 (m, 1H), 7.10 (s, 1H), 3.75 (s, 3H), 2.27 (s, 3H), 2.19 (s, 3H).

### Example 230: Preparation of Compound 582

### Step 1: N-[5-fluoro-4-[4-(1-methyl-4-piperidyl)phenoxy]-6-(o-tolyl)pyrimidin-2-yl]-1-methyl-pyrazole-4-sulfonamide

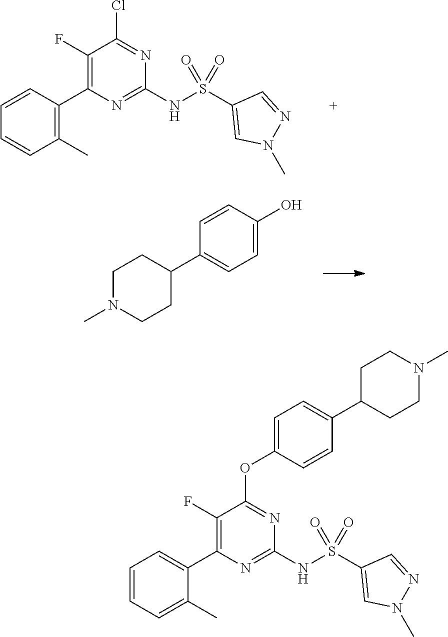

A mixture of 4-(1-methyl-4-piperidyl)phenol (approximately 32.57 mg, 0.1703 mmol), N-[4-chloro-5-fluoro-6-(o-tolyl)pyrimidin-2-yl]-1-methyl-pyrazole-4-sulfonamide (50 mg, 0.1310 mmol), and cesium carbonate (approximately 170.7 mg, 0.5240 mmol) in NMP (2.830 mL) was stirred at 110° C. for 22 hours and then cooled to room temperature. The solution was filtered and the resulting residues dissolved in 0.8 mL MeOH, and purified by reverse phase chromatography using a 15 minutes gradient of 25% MeCN in water to 75% MeCN (HCl modifier) to give N-[5-fluoro-4-[4-(1-methyl-4-piperidyl)phenoxy]-6-(o-tolyl)pyrimidin-2-yl]-1-methyl-pyrazole-4-sulfonamide (21 mg, 30%). ESI-MS m/z calc. 536.20056, found 537.0 (M+1)⁺; Retention time: 1.27 minutes; LC method A. ¹H NMR (400 MHz, DMSO-d₆) δ 11.62 (s, 1H), 9.91 (s, 1H), 7.61 (s, 1H), 7.48-7.31 (m, 8H), 7.05 (s, 1H), 3.75 (s, 3H), 3.51 (d, J=11.9 Hz, 2H), 3.08 (s, 2H), 2.91 (s, 1H), 2.80 (s, 3H), 2.26 (s, 3H), 1.95 (d, J=13.1 Hz, 4H).

### Example 231: Preparation of Compound 583

### Step 1: N-[5-fluoro-4-[3-(4-methylpiperazin-1-yl)phenoxy]-6-(o-tolyl)pyrimidin-2-yl]-1-methyl-pyrazole-4-sulfonamide

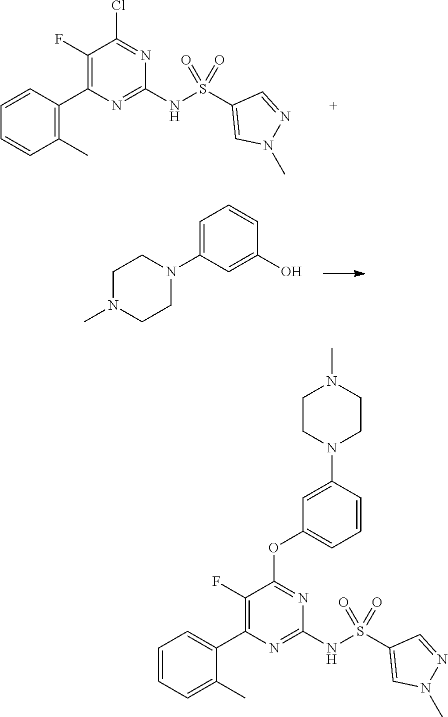

A mixture of 3-(4-methylpiperazin-1-yl)phenol (approximately 34.11 mg, 0.1703 mmol), N-[4-chloro-5-fluoro-6-(o-tolyl)pyrimidin-2-yl]-1-methyl-pyrazole-4-sulfonamide (50 mg, 0.1310 mmol), and cesium carbonate (approximately 170.7 mg, 0.5240 mmol) in NMP (2.830 mL) was stirred at 110° C. for 22 hours and then cooled to room temperature. The solution was filtered and the resulting residues dissolved in 0.8 mL MeOH, and purified by reverse phase chromatography using a 15 min gradient of 25% MeCN in water to 75% MeCN (HCl modifier) to give N-[5-fluoro-4-[3-(4-methylpiperazin-1-yl)phenoxy]-6-(o-tolyl)pyrimidin-2-yl]-1-methyl-pyrazole-4-sulfonamide (22.3 mg, 31%). ESI-MS m/z calc. 537.19586, found 538.0 (M+1)⁺; Retention time: 1.29 minutes; LC method A. ¹H NMR (400 MHz, DMSO-d₆) δ 10.40 (s, 1H), 7.52 (s, 1H), 7.47-7.41 (m, 2H), 7.40-7.32 (m, 3H), 7.16 (s, 1H), 7.09-7.02 (m, 2H), 6.87-6.83 (m, 1H), 4.16-3.79 (m, 4H), 3.74 (s, 3H), 3.27-3.04 (m, 4H), 2.81 (s, 3H), 2.27 (s, 3H).

### Example 232: Preparation of Compound 584

### Step 1: N-[5-fluoro-4-[4-(4-methylpiperazin-1-yl)phenoxy]-6-(o-tolyl)pyrimidin-2-yl]-1-methyl-pyrazole-4-sulfonamide

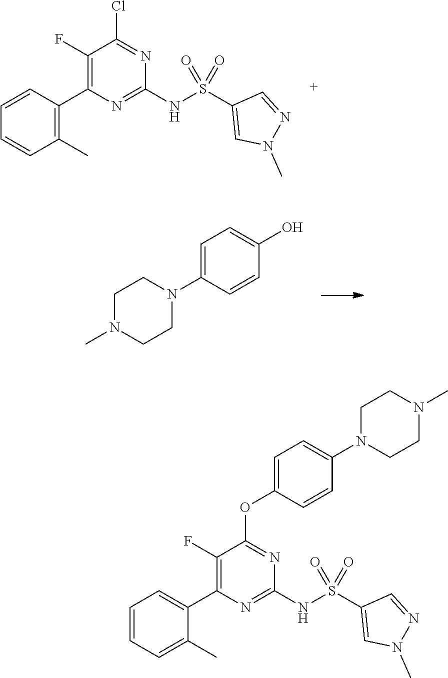 [SMILES: Cc1ccccc1-c1nc(NS(=O)(=O)c2cnn(C)c2)nc(Cl)c1F ; CN1CCN(c2ccc(O)cc2)CC1 ; Cc1ccccc1-c1nc(NS(=O)(=O)c2cnn(C)c2)nc(Oc2ccc(N3CCN(C)CC3)cc2)c1F]

A mixture of 4-(4-methylpiperazin-1-yl)phenol (approximately 32.74 mg, 0.1703 mmol), N-[4-chloro-5-fluoro-6-(o-tolyl)pyrimidin-2-yl]-1-methyl-pyrazole-4-sulfonamide (50mg, 0.1310 mmol), and cesium carbonate (approximately 170.7 mg, 0.5240 mmol) in NMP (2.830 mL) was stirred at 110° C. for 22 hours and then cooled to room temperature. The solution was filtered and the resulting residues dissolved in 0.8 mL MeOH, and purified by reverse phase chromatography using a 15 minute gradient of 25% MeCN in water to 75% MeCN (HCl modifier) to give N-[5-fluoro-4-[4-(4-methylpiperazin-1-yl)phenoxy]-6-(o-tolyl)pyrimidin-2-yl]-1-methyl-pyrazole-4-sulfonamide (31.7 mg, 45%). ESI-MS m/z calc. 537.19586, found 538.0 (M+1)⁺; Retention time: 1.24 minutes; LC method A.

### Example 233: Preparation of Compound 585

### Step 1: N-[4-[2-chloro-3-(4-methylpiperazin-1-yl)phenoxy]-5-fluoro-6-(o-tolyl)pyrimidin-2-yl]-1-methyl-pyrazole-4-sulfonamide

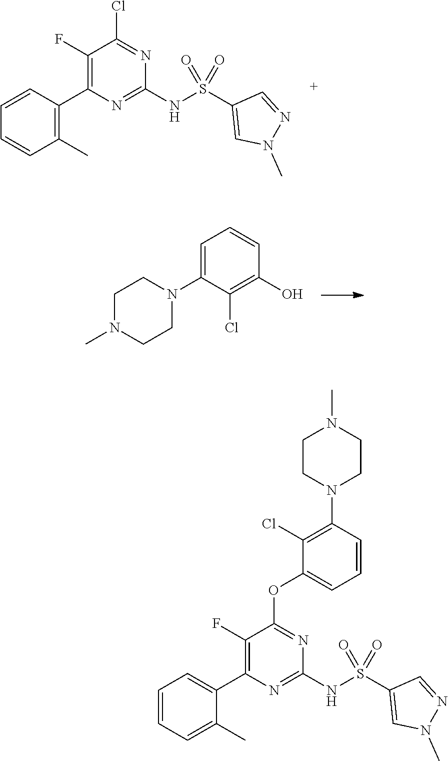 [SMILES: Cc1ccccc1-c1nc(NS(=O)(=O)c2cnn(C)c2)nc(Cl)c1F ; CN1CCN(c2cccc(O)c2Cl)CC1 ; Cc1ccccc1-c1nc(NS(=O)(=O)c2cnn(C)c2)nc(Oc2cccc(N3CCN(C)CC3)c2Cl)c1F]

A mixture of 2-chloro-3-(4-methylpiperazin-1-yl)phenol (approximately 38.61 mg, 0.1703 mmol), N-[4-chloro-5-fluoro-6-(o-tolyl)pyrimidin-2-yl]-1-methyl-pyrazole-4-sulfonamide (50 mg, 0.1310 mmol), and cesium carbonate (approximately 170.7 mg, 0.5240 mmol) in NMP (2.830 mL) was stirred at 110° C. for 22 hours and then cooled to room temperature. The solution was filtered and the resulting residues dissolved in 0.8 mL MeOH, and purified by reverse phase chromatography using a 15 minute gradient of 25% MeCN in water to 75% MeCN (HCl modifier) to give N-[4-[2-chloro-3-(4-methylpiperazin-1-yl)phenoxy]-5-fluoro-6-(o-tolyl)pyrimidin-2-yl]-1-methyl-pyrazole-4-sulfonamide (30.4 mg, 40%). ESI-MS m/z calc. 571.15686, found 572.0 (M+1)⁺; Retention time: 1.3 minutes; LC method A.

### Example 234: Preparation of Compound 586

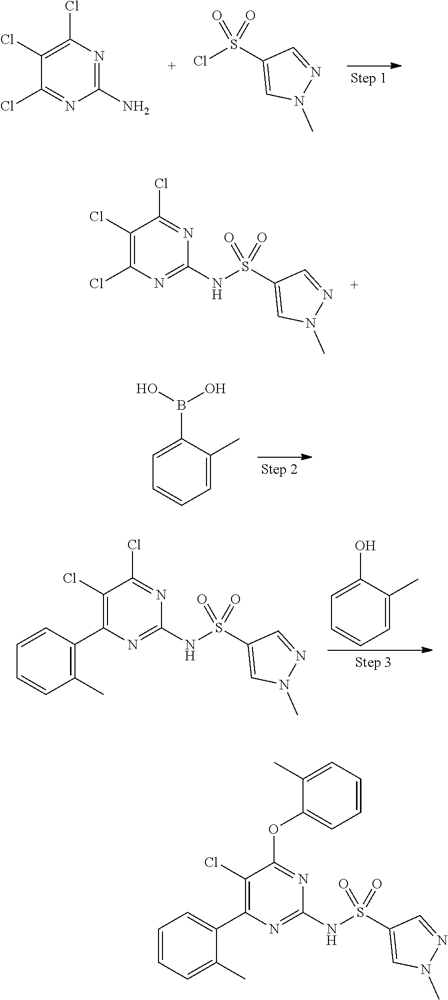

### Step 1: 1-Methyl-N-(4,5,6-trichloropyrimidin-2-yl)pyrazole-4-sulfonamide

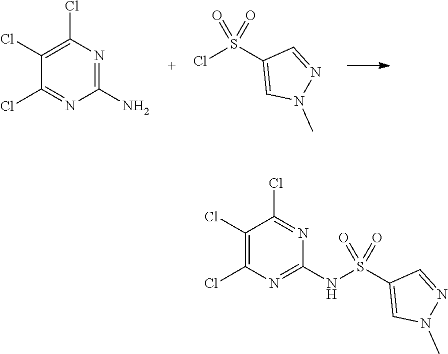

A DMF (30 mL) suspension of NaH (800 mg, 33.34 mmol) was cooled to 0° C. using an ice-water bath and it was treated with solid 4,5,6-trichloropyrimidin-2-amine (2 g, 10.08 mmol). The reaction mixture was stirred for 20 minutes and then treated with solid 1-methylpyrazole-4-sulfonyl chloride (2 g, 11.07 mmol). The reaction mixture was warmed to room temperature and stirred for 1 hour and then cooled to 0° C. HCl (30 mL of 1 M, 30.00 mmol) was added and the reaction mixture was stirred at 0° C. for 10 minutes upon which the product crystallized out. The reaction mixture was filtered, rinsed with cold water, and dried overnight under vacuum to give 1-methyl-N-(4,5,6-trichloropyrimidin-2-yl)pyrazole-4-sulfonamide (3.45 g, 99%)¹H NMR (400 MHz, DMSO-d₆) δ 12.49 (s, 1H), 8.41 (s, 1H), 7.90 (s, 1H), 3.88 (s, 3H). ESI-MS m/z calc. 340.9308, found 344.0 (M+3)*; Retention time. 1.22 minutes (LC method A).

### Step 2: N-[4,5-Dichloro-6-(o-tolyl)pyrimidin-2-yl]-1-methyl-pyrazole-4-sulfonamide

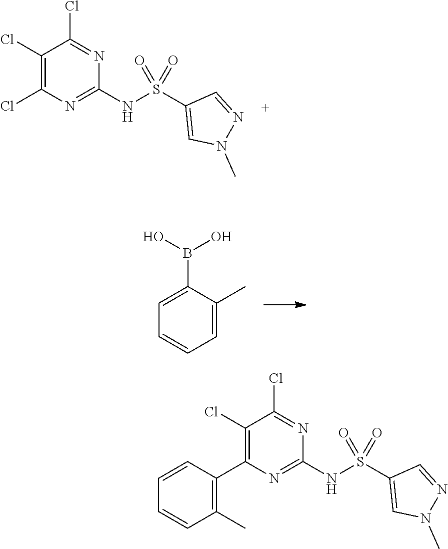

1-Methyl-N-(4,5,6-trichloropyrimidin-2-yl)pyrazole-4-sulfonamide (700 mg, 2.023 mmol), o-tolylboronic acid (287 mg, 2.111 mmol), tetrakis(triphenylphosphine)palladium (0) (115.5 mg, 0.09995 mmol), and 2 M sodium carbonate (3 mL of 2 M, 6.000 mmol) in 1,2-dimethoxyethane (7 mL) were combined in a microwave vial. The mixture was purged well with nitrogen, sealed and stirred for 15 hours at 75° C. The reaction mixture was diluted with water, extracted with ethyl acetate. The organic layer was dried over Na₂SO₄, concentrated, and purified on silica using a gradient of ethyl acetate/hexane. Product came out ˜50% ethyl acetate. The semi-clean product was purified on reverse phase HPLC (HCl modifier, 25-75% ACN-H₂O) to give N-[4,5-dichloro-6-(o-tolyl)pyrimidin-2-yl]-1-methyl-pyrazole-4-sulfonamide (396 mg, 49%). ESI-MS m/z calc. 397.0167, found 398.0 (M+1)⁺; Retention time: 1.65 minutes (LC method A).

### Step 3: N-[5-chloro-4-(2-methylphenoxy)-6-(o-tolyl)pyrimidin-2-yl]-1-methyl-pyrazole-4-sulfonamide

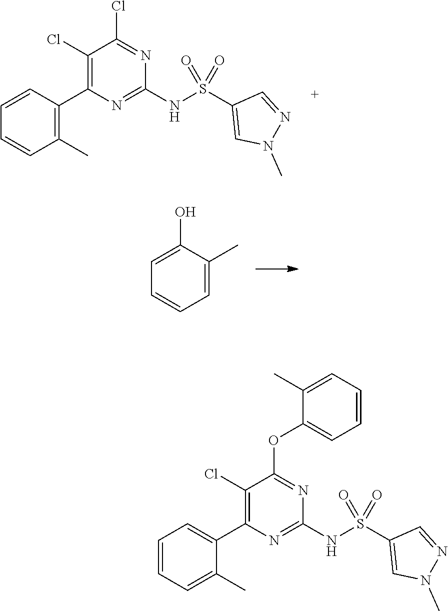

A mixture of o-cresol (approximately 17.65 mg, 31.93 μL, 0.1632 mmol), N-[4,5-dichloro-6-(o-tolyl)pyrimidin-2-yl]-1-methyl-pyrazole-4-sulfonamide (50 mg, 0.1255 mmol), and cesium carbonate (approximately 163.6 mg, 0.5020 mmol) in NMP (2.830 mL) was stirred at 110° C. for 22 hours and then cooled to room temperature. The solution was filtered and the resulting residue dissolved in 0.8 mL MeOH, and purified by reverse phase chromatography using a 15 min gradient of 25% MeCN in water to 75% MeCN (HCl modifier) to give N-[5-chloro-4-(2-methylphenoxy)-6-(o-tolyl)pyrimidin-2-yl]-1-methyl-pyrazole-4-sulfonamide (29.4 mg, 49%). ESI-MS m/z calc. 469.09753, found 470.0 (M+1)⁺; Retention time: 1.85 minutes; LC method A. ¹H NMR (400 MHz, DMSO-d₆) δ 11.78 (s, 1H), 7.47-7.44 (m, 1H), 7.44-7.38 (m, 4H), 7.37-7.33 (m, 2H), 7.33-7.28 (m, 2H), 7.05 (s, 1H), 3.75 (s, 3H), 2.22-2.13 (m, 6H).

### Example 235: Preparation of Compound 587

### Step 1: N-[5-chloro-4-[4-(1-methyl-4-piperidyl)phenoxy]-6-(o-tolyl)pyrimidin-2-yl]-1-methyl-pyrazole-4-sulfonamide

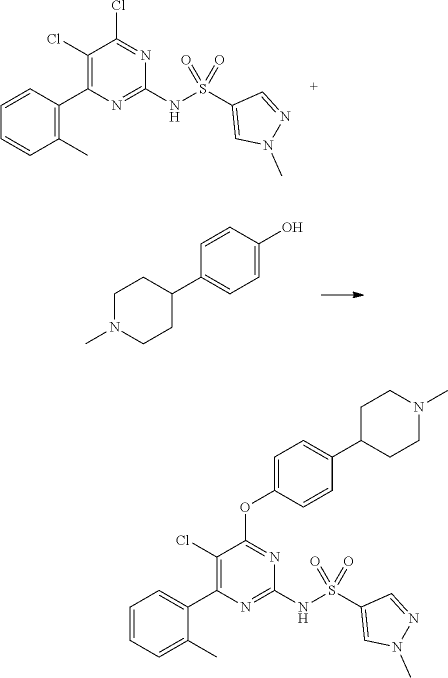 [SMILES: Cc1ccccc1-c1nc(NS(=O)(=O)c2cnn(C)c2)nc(Cl)c1Cl ; CN1CCC(c2ccc(O)cc2)CC1 ; Cc1ccccc1-c1nc(NS(=O)(=O)c2cnn(C)c2)nc(Oc2ccc(C3CCN(C)CC3)cc2)c1Cl]

A mixture of 4-(1-methyl-4-piperidyl)phenol (approximately 31.22 mg, 0.1632 mmol), N-[4,5-dichloro-6-(o-tolyl)pyrimidin-2-yl]-1-methyl-pyrazole-4-sulfonamide (50 mg, 0.1255 mmol), and cesium carbonate (approximately 163.6 mg, 0.5020 mmol) in NMP (2.830 mL) was stirred at 110° C. for 22 hours and then cooled to room temperature. The solution was filtered and the resulting residue dissolved in 0.8 mL MeOH, and purified by reverse phase chromatography using a 15 minute gradient of 25% MeCN in water to 75% MeCN (HCl modifier) to give N-[5-chloro-4-[4-(1-methyl-4-piperidyl)phenoxy]-6-(o-tolyl)pyrimidin-2-yl]-1 -methyl-pyrazole-4-sulfonamide (29.7 mg, 43%). ESI-MS m/z calc. 552.171, found 553.0 (M+1)⁺; Retention time: 1.32 minutes; LC method A. ¹H NMR (400 MHz, DMSO-d₆) δ 10.24 (s, 1H), 7.51 (s, 1H), 7.46-7.38 (m, 3H), 7.37-7.34 (m, 3H), 7.33-7.29 (m, 1H), 7.26 (d, J=7.4 Hz, 1H), 7.00 (s, 1H), 3.75 (s, 3H), 3.34-3.27 (m, 2H), 3.14-3.02 (m, 2H), 2.95-2.88 (m, 1H), 2.70 (s, 3H), 2.16 (s, 3H), 2.03 (s, 2H), 1.95-1.85 (m, 2H).

### Example 236: Preparation of Compound 588

### Step 1: N-[5-chloro-4-[3-(4-methylpiperazin-1-yl)phenoxy]-6-(o-tolyl)pyrimidin-2-yl]-1-methyl-pyrazole-4-sulfonamide

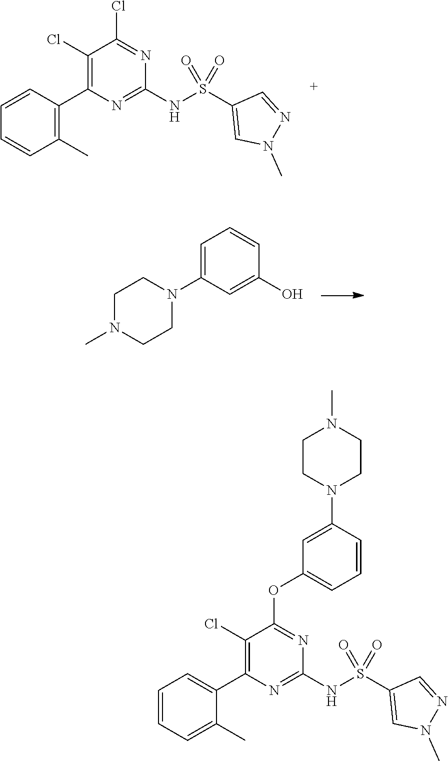 [SMILES: Cc1ccccc1-c1nc(NS(=O)(=O)c2cnn(C)c2)nc(Cl)c1Cl ; CN1CCN(c2cccc(O)c2)CC1 ; Cc1ccccc1-c1nc(NS(=O)(=O)c2cnn(C)c2)nc(Oc2cccc(N3CCN(C)CC3)c2)c1Cl]

A mixture of 3-(4-methylpiperazin-1-yl)phenol (approximately 32.68 mg, 0.1632 mmol), N-[4,5-dichloro-6-(o-tolyl)pyrimidin-2-yl]-1-methyl-pyrazole-4-sulfonamide (50 mg, 0.1255 mmol), and cesium carbonate (approximately 163.6 mg, 0.5020 mmol) in NMP (2.830 mL) was stirred at 110° C. for 22 hours and then cooled to room temperature. The solution was filtered and the resulting residue diluted with 0.8 mL MeOH, and purified by reverse phase chromatography using a 15 minute gradient of 25% MeCN in water to 75% MeCN (HCl modifier) to give N-[5-chloro-4-[3-(4-methylpiperazin-1-yl)phenoxy]-6-(o-tolyl)pyrimidin-2-yl]-1-methyl-pyrazole-4-sulfonamide (33.3 mg, 47%). ESI-MS m/z calc. 553.16626, found 554.0 (M+1)⁺; Retention time: 1.28 minutes; LC method A. ¹H NMR (400 MHz, DMSO-d₆) 10.37 (s, 1H), 7.44 (d, J=5.2 Hz, 2H), 7.40 (d, J=7.3 Hz, 1H), 7.37-7.34 (m, 1H), 7.31 (d, J=7.3 Hz, 1H), 7.28-7.23 (m, 1H), 7.11 (s, 1H), 7.07 (s, 1H), 7.04 (s, 1H), 6.86-6.81 (m, 1H), 4.18-3.82 (m, 4H), 3.74 (s, 3H), 3.28-3.07 (m, 4H), 2.82 (s, 3H), 2.16 (s, 3H).

### Example 237: Preparation of Compound 589

### Step 1: N-[5-chloro-4-[2-chloro-3-(4-methylpiperazin-1-yl)phenoxy]-6-(o-tolyl)pyrimidin-2-yl]-1-methyl-pyrazole-4-sulfonamide

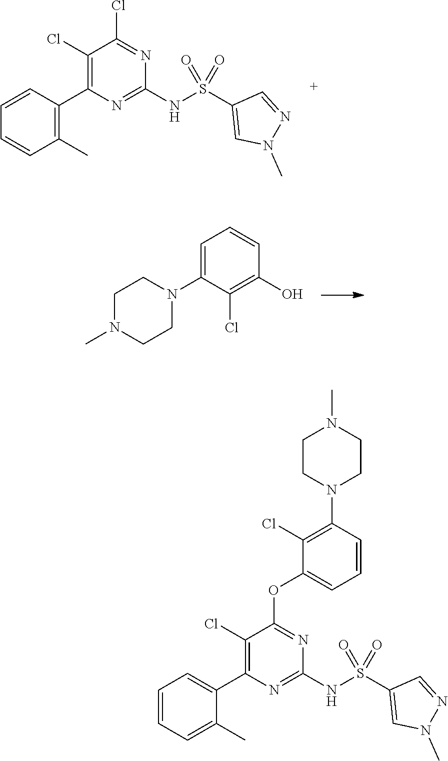 [SMILES: Cc1ccccc1-c1nc(NS(=O)(=O)c2cnn(C)c2)nc(Cl)c1Cl ; CN1CCN(c2cccc(O)c2Cl)CC1 ; Cc1ccccc1-c1nc(NS(=O)(=O)c2cnn(C)c2)nc(Oc2cccc(N3CCN(C)CC3)c2Cl)c1Cl]

A mixture of 2-chloro-3-(4-methylpiperazin-1-yl)phenol (approximately 37.00 mg, 0.1632 mmol), N-[4,5-dichloro-6-(o-tolyl)pyrimidin-2-yl]-1-methyl-pyrazole-4-sulfonamide (50 mg, 0.1255 mmol), and cesium carbonate (approximately 163.6 mg, 0.5020 mmol) in NMP (2.830 mL) was stirred at 110° C. for 22 hours and then cooled to room temperature. The solution was filtered and the resulting residue dissolved in 0.8 mL MeOH, and purified by reverse phase chromatography using a 15 minute gradient of 25% MeCN in water to 75% MeCN (HCl modifier) to give N-[5-chloro-4-[2-chloro-3-(4-methylpiperazin-1-yl)phenoxy]-6-(o-tolyl)pyrimidin-2-yl]-1-methyl-pyrazole-4-sulfonamide (26.0 mg, 34%). ESI-MS m/z calc. 587.1273, found 588.0 (M+1)⁺; Retention time: 1.34 minutes; LC method A. ¹H NMR (400 MHz, DMSO-d₆) δ 11.02-10.10 (m, 1H), 7.60 (s, 1H), 7.58-7.51 (m, 1H), 7.45-7.39 (m, 1H), 7.39-7.32 (m, 2H), 7.29 (t, J=6.8 Hz, 3H), 7.00 (s, 1H), 3.79 (s, 3H), 3.67-3.44 (m, 4H), 3.28-2.98 (m, 4H), 2.85 (s, 3H), 2.16 (s, 3H).

### Example 238: Preparation of Compound 590

### Step 1: N-[5-chloro-4-[4-(4-methylpiperazin-1-yl)phenoxy]-6-(o-tolyl)pyrimidin-2-yl]-1-methyl-pyrazole-4-sulfonamide

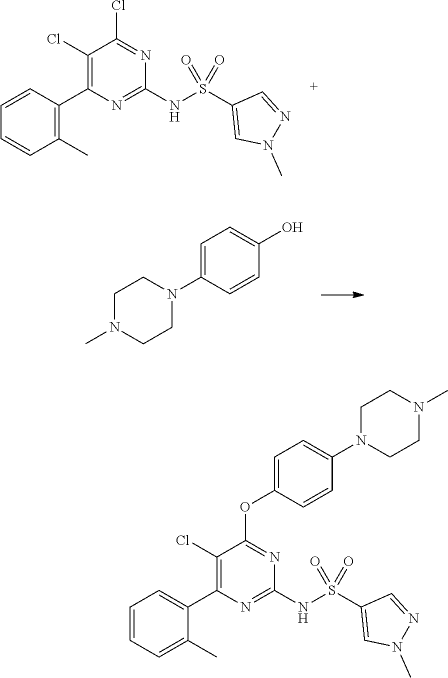 [SMILES: Cc1ccccc1-c1nc(NS(=O)(=O)c2cnn(C)c2)nc(Cl)c1Cl ; CN1CCN(c2ccc(O)cc2)CC1 ; Cc1ccccc1-c1nc(NS(=O)(=O)c2cnn(C)c2)nc(Oc2ccc(N3CCN(C)CC3)cc2)c1Cl]

A mixture of 4-(4-methylpiperazin-1-yl)phenol (approximately 31.38 mg, 0.1632 mmol), N-[4,5-dichloro-6-(o-tolyl)pyrimidin-2-yl]-1-methyl-pyrazole-4-sulfonamide (50 mg, 0.1255 mmol), and cesium carbonate (approximately 163.6 mg, 0.5020 mmol) in NMP (2.830 mL) was stirred at 110° C. for 22 hours and then cooled to room temperature. The solution was filtered and the resulting residues dissolved in 0.8 mL MeOH, and purified by reverse phase chromatography using a 15 minute gradient of 25% MeCN in water to 75% MeCN (HCl modifier) to give N-[5-chloro-4-[4-(4-methylpiperazin-1-yl)phenoxy]-6-(o-tolyl)pyrimidin-2-yl]-1-methyl-pyrazole-4-sulfonamide (20.5 mg, 29%). ESI-MS m/z calc. 553.16626, found 554.0 (M+1)⁺; Retention time: 1.27 minutes; LC method A. ¹H NMR (400 MHz, DMSO-d₆) δ 11.77 (s, 1H), 10.67 (s, 1H), 7.53 (s, 1H), 7.51-7.24 (m, 7H), 7.17 (d, J=14.5 Hz, 2H), 3.81 (d, J=47.2 Hz, 7H), 3.20 (s, 4H), 2.83 (s, 3H), 2.15 (s, 3H).

### Example 239: Preparation of Compound 591

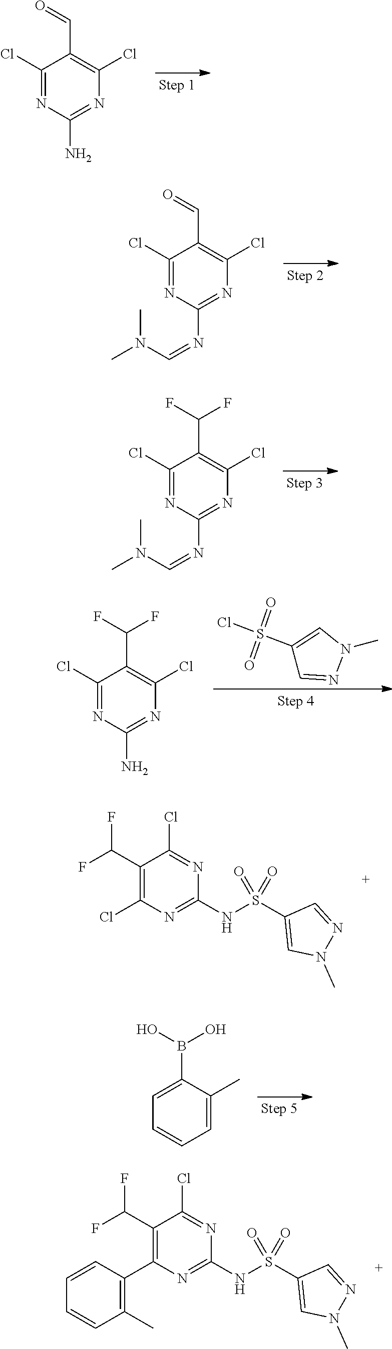

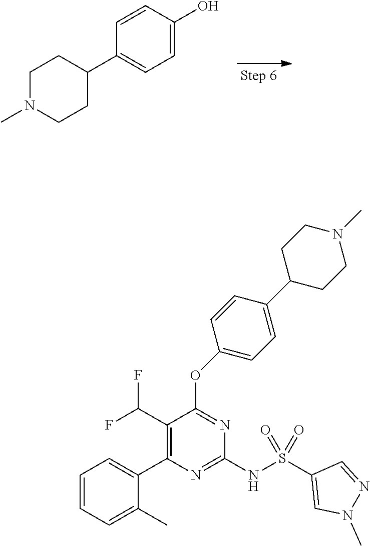 [SMILES: CN1CCC(c2ccc(O)cc2)CC1 ; Cc1ccccc1-c1nc(NS(=O)(=O)c2cnn(C)c2)nc(Oc2ccc(C3CCN(C)CC3)cc2)c1C(F)F]

### Step 1: N′-(4,6-Dichloro-5-formyl-pyrimidin-2-yl)-N,N-dimethyl-formamidine

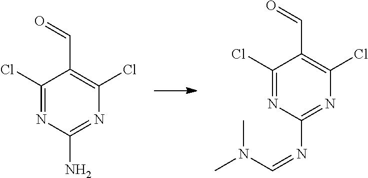

A solution of DMF (65 mL, 839 mmol) in chloroform (1.4 L) was treated slowly with oxalyl chloride (36 mL, 413 mmol). After about 15 minutes, 2-amino-4,6-dichloro-pyrimidine-5-carbaldehyde (53.76 g, 280 mmol) was added and the reaction was left to stir at room temperature for about 3.5 hours. Insoluble material from the reaction mixture was removed by filtration over Celite which was washed with chloroform (about 300 mL). The reaction was then quenched by being added to an Erlenmeyer flask containing 5% aqueous sodium bicarbonate (about 1.5 L) and left to stir vigorously at room temperature for 10-15 minutes. The biphasic mixture was transferred to a 4.0-L separatory funnel and the layers were separated. The organic layer was then dried over sodium sulfate, filtered and concentrated under reduced pressure. The residue (about 80 g of an off-white slightly gummy solid) was split in half and purified by silica gel chromatography on a 330 g column, eluting from 0% to 25% ethyl acetate in dichloromethane. All fractions containing product were combined and concentrated under reduced pressure. Once most of the dichloromethane was removed, a voluminous solid crashed out from the remaining ethyl acetate. This solid was filtered off, washed with ethyl acetate and dried under high vacuum to afford a first lot of N′-(4,6-dichloro-5-formyl-pyrimidin-2-yl)-N,N-dimethyl-formamidine (9.82 g, 13% yield) as a beige yellow solid. ESI-MS m/z calc. 246.0075, found 247.1 (M+1)⁺; Retention time: 1.31 minutes (LC method C). The second half of crude product was purified according to the exact same procedure described above to provide a second lot of N′-(4,6-dichloro-5-formyl-pyrimidin-2-yl)-N,N-dimethyl-formamidine (11.59 g, 16% yield) as a pale yellow solid. ESI-MS m/z calc. 246.0075, found 247.1 (M+1)⁺; Retention time: 1.31 minutes (LC method C). Total amount of 21.41 g (29% yield).

### Step 2: N′-[4,6-Dichloro-5-(difluoromethyl)pyrimidin-2-yl]-N,N-dimethyl-formamidine

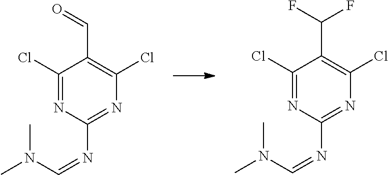 [SMILES: CN(C)/C=N\c1nc(Cl)c(C=O)c(Cl)n1 ; CN(C)/C=N\c1nc(Cl)c(C(F)F)c(Cl)n1]

A solution of N′-(4,6-dichloro-5-formyl-pyrimidin-2-yl)-N,N-dimethyl-formamidine (21.41 g, 86.65 mmol) in dichloroethane (500 mL) was treated with (dimethylamino)sulfur trifluoride (26 mL, 266 mmol). The reaction was heated in an oil bath at 30° C. for 5 hours. The reaction was quenched by being added portionwise into a 2.0-L Erlenmeyer flask containing 5% aqueous sodium bicarbonate (1.0-L) stirred vigorously at room temperature for about 15 minutes (Some solid sodium bicarbonate was added at the end to ensure a basic pH). The mixture was transferred to a 2.0-L separatory funnel, the layers were then separated and the aqueous layer was extracted with dichloromethane (2×200 mL). The combined organic layers were dried over sodium sulfate, filtered and concentrated under reduced pressure to afford crude N′-[4,6-dichloro-5-(difluoromethyl)pyrimidin-2-yl]-N,N-dimethyl-formamidine (18.46 g, 61.2% purity by LCMS, 48% yield) as a yellow, thick oil that solidified on standing. ESI-MS m/z calc. 268.0094, found 269.1 (M+1)⁺; Retention time: 1.51 minutes (LC method C).

### Step 3: 4,6-Dichloro-5-(difluoromethyl)pyrimidin-2-amine

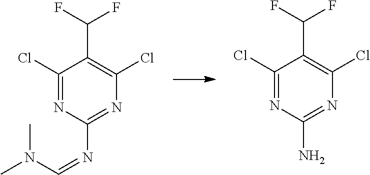 [SMILES: CN(C)/C=N\c1nc(Cl)c(C(F)F)c(Cl)n1 ; Nc1nc(Cl)c(C(F)F)c(Cl)n1]

Crude N′-[4,6-dichloro-5-(difluoromethyl)pyrimidin-2-yl]-N,N-dimethyl-formamidine (18.46 g, 61.2% purity, 42.0 mmol) was taken up in isopropanol (165 mL) and concentrated hydrochloric acid (33 mL, 396 mmol) and the mixture was heated in an oil bath at 60° C. for 60 minutes. Once cooled to room temperature, the crude mixture was transferred to a separatory funnel with water (1.5 L) and ethyl acetate (500 mL). The layers were separated, and the aqueous layer was extracted again with ethyl acetate (2×250 mL). The combined organic layer was extracted with brine (2×250 mL), dried over sodium sulfate, filtered and concentrated under reduced pressure. The residue was purified by silica gel chromatography on a 220 g column, (eluting from 0% to 30% ethyl acetate in heptanes). The pure fractions were combined and concentrated under reduced pressure. The solid was triturated in a mixture of ethyl acetate and heptanes (15% ethyl acetate in heptanes, 20 mL), filtered, washed (15% ethyl acetate in heptanes, 20 mL) and dried under high vacuum to afford 4,6-dichloro-5-(difluoromethyl)pyrimidin-2-amine (2.892 g, 97.5% purity, 31% yield) as a white solid. ¹H NMR (300 MHz, DMSO-d₆) δ ppm 7.19 (t, J=52.2 Hz, 1H), 8.13 (s, 2H). ¹⁹F NMR (282 MHz, DMSO-d₆) δ ppm −111.92 (d, J=53.1 Hz, 2F). ESI-MS m/z calc. 212.9672, found 214.0 (M+1)⁺+; Retention time: 1.99 minutes, (LC method M). Impure fractions from the chromatography were combined and concentrated under reduced pressure. The solid was triturated in a mixture of ethyl acetate and heptanes (15% ethyl acetate in heptanes, 20 mL), filtered, washed (15% ethyl acetate in heptanes, 20 mL) and dried under high vacuum to afford a second lot of 4,6-dichloro-5-(difluoromethyl)pyrimidin-2-amine (1.237 g, 90.2% purity, 12% yield) as a white solid.

### Step 4: N-[4,6-Dichloro-5-(difluoromethyl)pyrimidin-2-yl]-1-methyl-pyrazole-4-sulfonamide

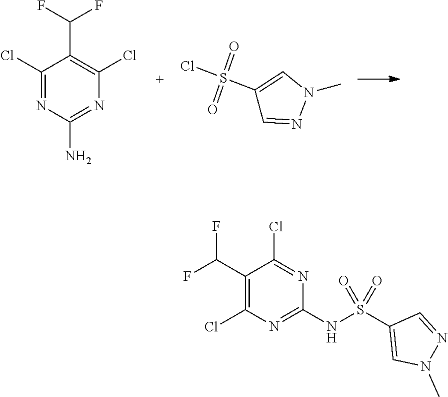

A DMF (11 mL) mixture of sodium hydride (282.1 mg, 11.76 mmol) was treated with 4,6-dichloro-5-(difluoromethyl)pyrimidin-2-amine (651.0 mg, 3.042 mmol) at 0° C. and the reaction mixture was stirred for 30 minutes. 1-Methylpyrazole-4-sulfonyl chloride (663.3 mg, 3.672 mmol) was added and the reaction mixture was warmed to room temperature and stirred for 1 hour and then cooled to 0° C. using an ice-water bath. HCl (6.5 mL of 2 M, 13.00 mmol) was added, upon which the product crystallized out. The reaction mixture was filtered and the solid was washed with cold water (1.5 mL) followed by cold MeOH (2 mL). The solid was dried under vacuum to give N-[4,6-dichloro-5-(difluoromethyl)pyrimidin-2-yl]-1-methyl-pyrazole-4-sulfonamide (1.0095 g, 93%) ESI-MS m/z calc. 356.96655, found 358.02 (M+1)⁺; Retention time: 1.31 minutes, (LC method A).

### Step 5: N-[4-chloro-5-(difluoromethyl)-6-(o-tolyl)pyrimidin-2-yl]-1-methyl-pyrazole-4-sulfonamide

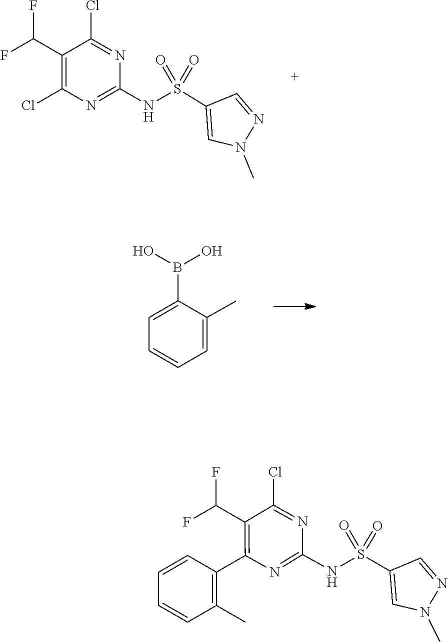

A dioxane (4.5 mL) mixture of o-tolylboronic acid (100.1 mg, 0.7363 mmol), N-[4,6-dichloro-5-(difluoromethyl)pyrimidin-2-yl]-1-methyl-pyrazole-4-sulfonamide (530.3 mg, 1.481 mmol), Pd(PPh₃)₄ (171.1 mg, 0.1481 mmol), and K₂CO₃ (1.5 mL of 2 M, 3.000 mmol) was sparged with nitrogen for 1 minute and then microwaved at 120° C. for 30 minutes and then cooled to room temperature. The solution was filtered and the filtrate dissolved in 0.8 mL MeOH, and purified by reverse phase chromatography using a 15 minute gradient of 1% MeCN in water to 99% MeCN (HCl modifier) to give N-[4-chloro-5-(difluoromethyl)-6-(o-tolyl)pyrimidin-2-yl]-1-methyl-pyrazole-4-sulfonamide (86.7 mg, 28%) ESI-MS m/z calc. 413.0525, found 414.1 (M+1)⁺; Retention time: 1.61 minutes, (LC method A).

### Step 6: N-[5-(difluoromethyl)-4-[4-(1-methyl-4-piperidyl)phenoxy]-6-(o-tolyl)pyrimidin-2-yl]-1-methyl-pyrazole-4-sulfonamide

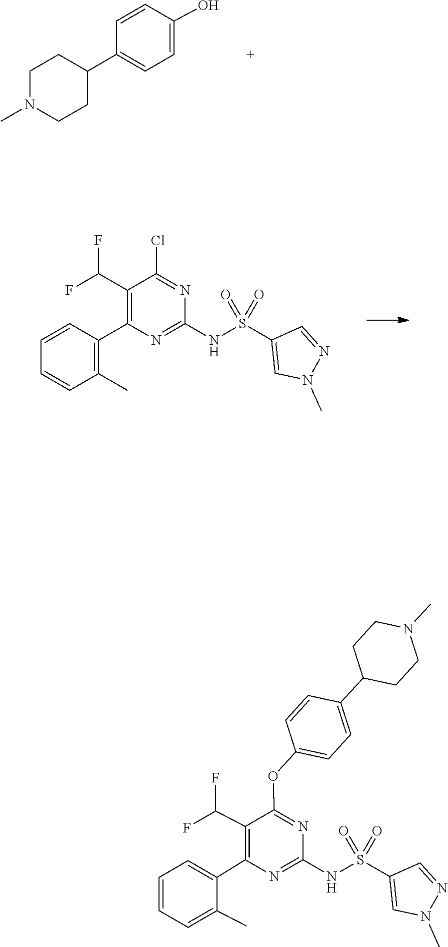 [SMILES: CN1CCC(c2ccc(O)cc2)CC1 ; Cc1ccccc1-c1nc(NS(=O)(=O)c2cnn(C)c2)nc(Cl)c1C(F)F ; Cc1ccccc1-c1nc(NS(=O)(=O)c2cnn(C)c2)nc(Oc2ccc(C3CCN(C)CC3)cc2)c1C(F)F]

A NMP (0.7 mL) mixture of 4-(1-methyl-4-piperidyl)phenol (acetate salt) (approximately 6.072 mg, 0.02416 mmol), N-[4-chloro-5-(difluoromethyl)-6-(o-tolyl)pyrimidin-2-yl]-1-methyl-pyrazole-4-sulfonamide (10 mg, 0.02416 mmol), and Cs₂CO₃ (approximately 31.49 mg, 0.09664 mmol) was heated at 110° C. for 16 hours and then cooled to room temperature. The solution was filtered and the filtrate dissolved in 0.8 mL MeOH, and purified by reverse phase chromatography using a 15 minutes gradient of 1% MeCN in water to 99% MeCN (HCl modifier) to give N-[5-(difluoromethyl)-4-[4-(1-methyl-4-piperidyl)phenoxy]-6-(o-tolyl)pyrimidin-2-yl]-1-methyl-pyrazole-4-sulfonamide (hydrochloride salt) (5.9 mg, 40%). ESI-MS m/z calc. 568.2068, found 569.32 (M+1)⁺; Retention time: 1.33 minutes; LC method A. ¹H NMR (400 MHz, DMSO-d₆) δ 10.11 (s, 1H), 7.53-7.27 (m, 8H), 7.21 (d, J=7.5 Hz, 1H), 6.97 (s, 1H), 6.69 (t, J=52.9 Hz, 1H), 3.73 (s, 3H), 3.51 (d, J=12.2 Hz, 2H), 3.17-2.99 (m, 2H), 2.99-2.85 (m, 1H), 2.79 (d, J=4.7 Hz, 3H), 2.13 (s, 3H), 2.10-1.90 (m, 4H).

### Example 240: Preparation of Compound 592

### Step 1: N-[5-(difluoromethyl)-4-[2-fluoro-4-(1-methyl-4-piperidyl)phenoxy]-6-(o-tolyl)pyrimidin-2-yl]-1-methyl-pyrazole-4-sulfonamide

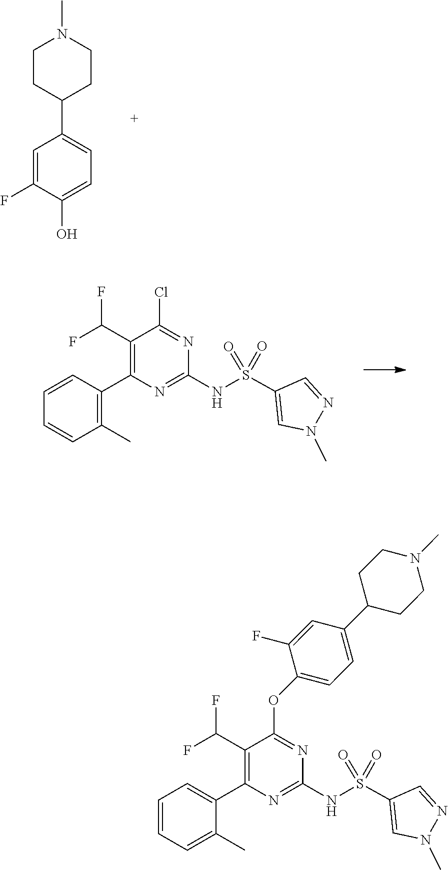 [SMILES: CN1CCC(c2ccc(O)c(F)c2)CC1 ; Cc1ccccc1-c1nc(NS(=O)(=O)c2cnn(C)c2)nc(Cl)c1C(F)F ; Cc1ccccc1-c1nc(NS(=O)(=O)c2cnn(C)c2)nc(Oc2ccc(C3CCN(C)CC3)cc2F)c1C(F)F]

A NMP (0.7 mL) mixture of 2-fluoro-4-(1-methyl-4-piperidyl)phenol (approximately 5.056 mg, 0.02416 mmol), N-[4-chloro-5-(difluoromethyl)-6-(o-tolyl)pyrimidin-2-yl]-1-methyl-pyrazole-4-sulfonamide (10 mg, 0.02416 mmol), and Cs₂CO₃ (approximately 31.49 mg, 0.09664 mmol) was heated at 110° C. for 16 hours and then cooled to room temperature. The solution was filtered, and the filtrate dissolved in 0.8 mL MeOH, and purified by reverse phase chromatography using a 15 minute gradient of 1% MeCN in water to 99% MeCN (HCl modifier) to give N-[5-(difluoromethyl)-4-[2-fluoro-4-(1-methyl-4-piperidyl)phenoxy]-6-(o-tolyl)pyrimidin-2-yl]-1-methyl-pyrazole-4-sulfonamide (hydrochloride salt). ESI-MS m/z calc. 586.1974, found 587.29 (M+1)⁺; Retention time: 1.35 minutes; LC method A.

### Example 241: Preparation of Compound 593

### Step 1: 2-Fluoro-3-(1-methyl-4-piperidyl)phenol

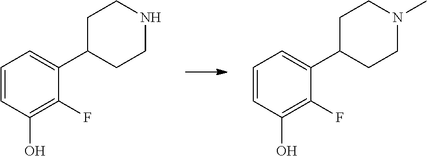

To a solution of 2-fluoro-3-(piperidin-4-yl)phenol trifluoroacetic acid salt (14.10 g, 45.48 mmol) in methanol (100 mL) and 37% aqueous formaldehyde solution (20 mL, 240 mmol) was added 10% palladium on carbon (2.7 g) and the mixture was stirred under hydrogen atmosphere at 50 psi for 1 hour. The reaction mixture was filtered and concentrated. Saturated aqueous sodium bicarbonate solution (70 mL) and ethyl acetate (300 mL) were added. The organic layer was washed with brine, dried over sodium sulfate and concentrated to give 2-fluoro-3-(N-methylpiperidin-4-yl)phenol (7.2 g, 76%) as an off-white solid. ¹H NMR (250 MHz, CDCl₃) δ (ppm): 6.60-7.05 (m, 3H), 2.95-3.10 (m, 2H), 2.75-2.90 (m, 1H), 2.34 (s, 3H), 2.05-2.15 (m, 2H), 1.70-1.98 (m, 4H). ESI-MS m/z calc. 209.1216, found 210.2 (M+1)⁺; Retention time: 1.89 minutes.

### Step 2: N-[5-(difluoromethyl)-4-[2-fluoro-3-(1-methyl-4-piperidyl)phenoxy]-6-(o-tolyl)pyrimidin-2-yl]-1-methyl-pyrazole-4-sulfonamide

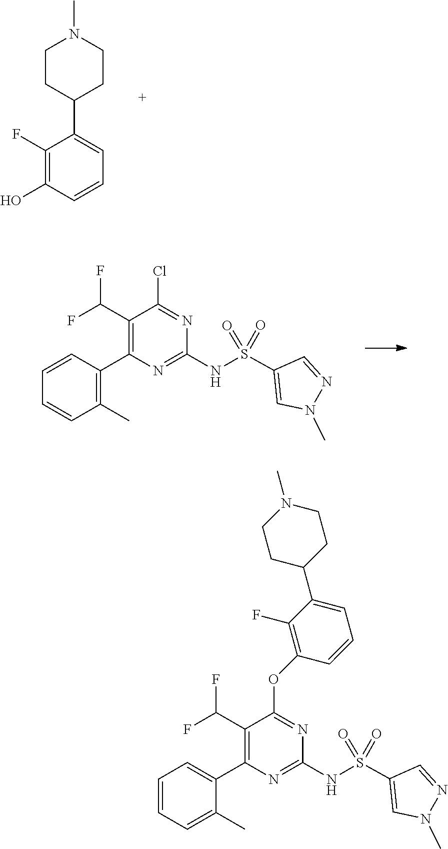 [SMILES: CN1CCC(c2cccc(O)c2F)CC1 ; Cc1ccccc1-c1nc(NS(=O)(=O)c2cnn(C)c2)nc(Cl)c1C(F)F ; Cc1ccccc1-c1nc(NS(=O)(=O)c2cnn(C)c2)nc(Oc2cccc(C3CCN(C)CC3)c2F)c1C(F)F]

A NMP (0.7 mL) mixture of 2-fluoro-3-(1-methyl-4-piperidyl)phenol (approximately 5.056 mg, 0.02416 mmol), N-[4-chloro-5-(difluoromethyl)-6-(o-tolyl)pyrimidin-2-yl]-1-methyl-pyrazole-4-sulfonamide (10 mg, 0.02416 mmol), and Cs₂CO₃ (approximately 31.49 mg, 0.09664 mmol) was heated at 110° C. for 16 hours and then cooled to room temperature. The solution was filtered, and the filtrate dissolved in 0.8 mL MeOH, and purified by reverse phase chromatography using a 15 minute gradient of 1% MeCN in water to 99% MeCN (HCl modifier) to give N-[5-(difluoromethyl)-4-[2-fluoro-3-(1-methyl-4-piperidyl)phenoxy]-6-(o-tolyl)pyrimidin-2-yl]-1-methyl-pyrazole-4-sulfonamide (hydrochloride salt). ESI-MS m/z calc. 586.1974, found 587.29 (M+1)⁺; Retention time: 1.34 minutes; LC method A.

### Example 242: Preparation of Compound 594

### Step 1: N-[5-(difluoromethyl)-4-[2-fluoro-3-(4-methylpiperazin-1-yl)phenoxy]-6-(o-tolyl)pyrimidin-2-yl]-1-methyl-pyrazole-4-sulfonamide

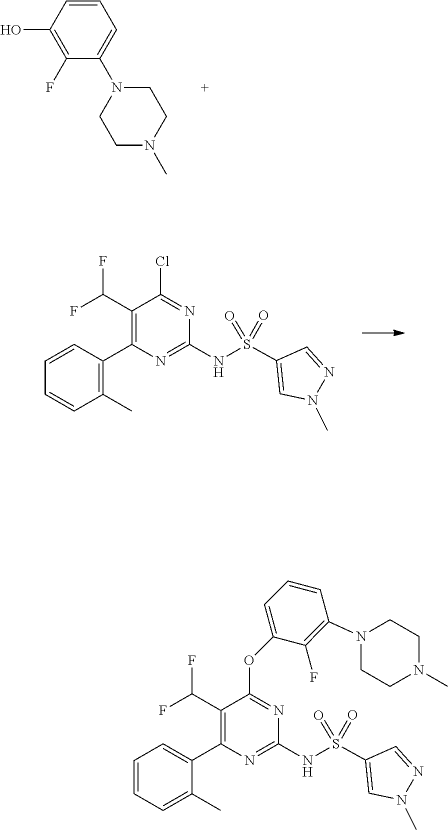 [SMILES: CN1CCN(c2cccc(O)c2F)CC1 ; Cc1ccccc1-c1nc(NS(=O)(=O)c2cnn(C)c2)nc(Cl)c1C(F)F ; Cc1ccccc1-c1nc(NS(=O)(=O)c2cnn(C)c2)nc(Oc2cccc(N3CCN(C)CC3)c2F)c1C(F)F]

A NMP (0.7 mL) mixture of 2-fluoro-3-(4-methylpiperazin-1-yl)phenol (approximately 5.080 mg, 0.02416 mmol), N-[4-chloro-5-(difluoromethyl)-6-(o-tolyl)pyrimidin-2-yl]-1-methyl-pyrazole-4-sulfonamide (10 mg, 0.02416 mmol), and Cs₂CO₃ (approximately 31.49 mg, 0.09664 mmol) was heated at 110° C. for 16 hours and then cooled to room temperature. The solution was filtered, and the filtrate dissolved in 0.8 mL MeOH, and purified by reverse phase chromatography using a 15 minute gradient of 1% MeCN in water to 99% MeCN (HCl modifier) to give N-[5-(difluoromethyl)-4-[2-fluoro-3-(4-methylpiperazin-1-yl)phenoxy]-6-(o-tolyl)pyrimidin-2-yl]-1-methyl-pyrazole-4-sulfonamide (hydrochloride salt). ESI-MS m/z calc. 587.1926, found 588.28 (M+1)⁺; Retention time: 1.33 minutes; LC method A.

### Example 243: Preparation of Compound 595

### Step 1: N-[5-(difluoromethyl)-4-[4-(4-methylpiperazin-1-yl)phenoxy]-6-(o-tolyl)pyrimidin-2-yl]-1-methyl-pyrazole-4-sulfonamide

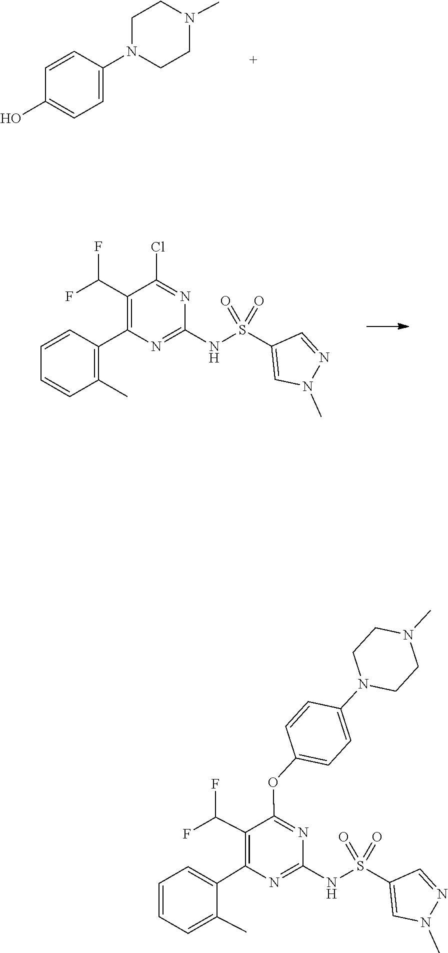 [SMILES: CN1CCN(c2ccc(O)cc2)CC1 ; Cc1ccccc1-c1nc(NS(=O)(=O)c2cnn(C)c2)nc(Cl)c1C(F)F ; Cc1ccccc1-c1nc(NS(=O)(=O)c2cnn(C)c2)nc(Oc2ccc(N3CCN(C)CC3)cc2)c1C(F)F]

A NMP (0.7 mL) mixture of 4-(4-methylpiperazin-1-yl)phenol (approximately 4.645 mg, 0.02416 mmol), N-[4-chloro-5-(difluoromethyl)-6-(o-tolyl)pyrimidin-2-yl]-1-methyl-pyrazole-4-sulfonamide (10 mg, 0.02416 mmol), and Cs₂CO₃ (approximately 31.49 mg, 0.09664 mmol) was heated at 110° C. for 16 hours and then cooled to room temperature. The solution was filtered, and the filtrate dissolved in 0.8 mL MeOH, and purified by reverse phase chromatography using a 15 minute gradient of 1% MeCN in water to 99% MeCN (HCl modifier) to give N-[5-(difluoromethyl)-4-[4-(4-methylpiperazin-1-yl)phenoxy]-6-(o-tolyl)pyrimidin-2-yl]-1-methyl-pyrazole-4-sulfonamide (hydrochloride salt). ESI-MS m/z calc. 569.2021, found 570.31 (M+1)⁺; Retention time: 1.29 minutes; LC method A.

### Example 244: Preparation of Compounds 596

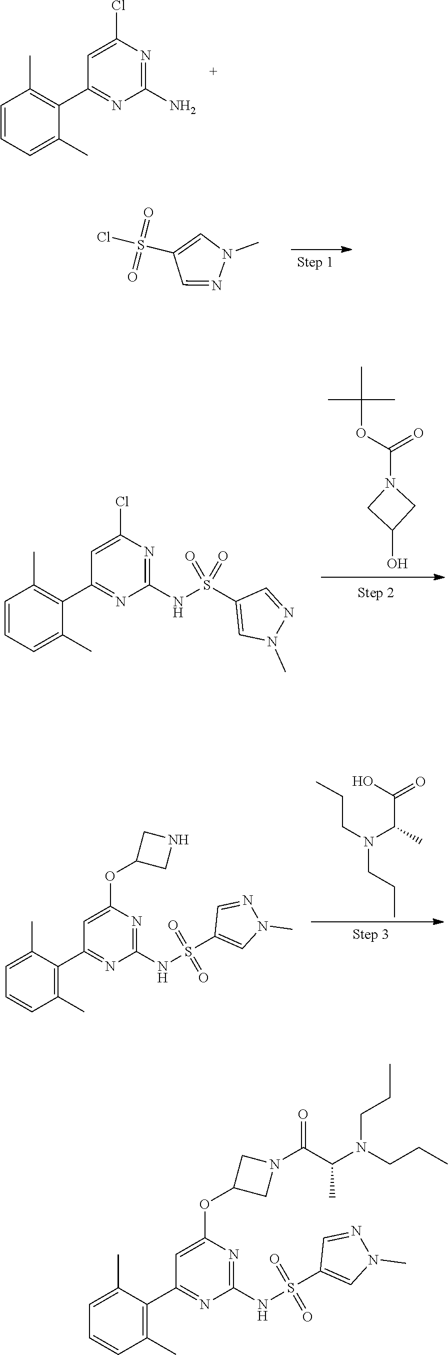

### Step 1: N-[4-chloro-6-(2,6-dimethylphenyl)pyrimidin-2-yl]-1-methyl-pyrazole-4-sulfonamide

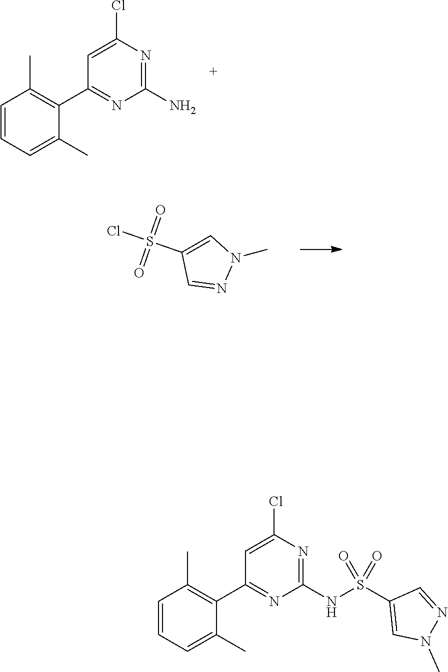

To a solution of 4-chloro-6-(2,6-dimethylphenyl)pyrimidin-2-amine (hydrochloride salt) (18 g, 66.63 mmol) and 1-methylpyrazole-4-sulfonyl chloride (approximately 15.04 g, 83.29 mmol) in 2-MeTHF (216.0 mL) at 0° C. was slowly added 1,1-dimethylpropoxylithium (approximately 75.23 mL of 3.1 M, 233.2 mmol) dropwise. The mixture was allowed to warm to ambient temperature and stirred for 18 hours. The mixture was heated to 45° C. Additional 1-methylpyrazole-4-sulfonyl chloride (approximately 3.009 g, 16.66 mmol) was added and the reaction was stirred at 45° C. for 30 minutes. Heat was removed and the reaction was stirred at ambient temperature for an additional 2 hours. The slurry was filtered using a M frit and washed several times with 50 mL of MTBE. The filtrate was diluted with 300 mL of water and the organic phase separated. The organic phase was extracted with 200 mL of water and the two aqueous phases combined. The aqueous phase was filtered through Celite and acidified with HCl (approximately 16.66 mL of 6 M, 99.94 mmol) affording a thick yellow oil. The mixture was extracted with 400 mL of EtOAc. The organic phase was washed with 200 mL of brine, dried over MgSO₄, filtered and concentrated in vacuo affording a light yellow foam. N-[4-chloro-6-(2,6-dimethylphenyl)pyrimidin-2-yl]-1-methyl-pyrazole-4-sulfonamide (13.2 g, 52%). H NMR (400 MHz, DMSO-d₆) δ 12.13 (s, 1H), 8.29 (d, J=0.6 Hz, 1H), 7.77 (d, J=0.7 Hz, 1H), 7.33 (s, 1H), 7.27 (dd, J=8.1, 7.0 Hz, 1H), 7.20-7.10 (m, 2H), 3.83 (s, 3H), 1.97 (s, 6H). ESI-MS m/z calc. 377.07132, found 378.0 (M+1)⁺; Retention time: 1.55 minutes. (LC method A).

### Step 2: N-[4-(azetidin-3-yloxy)-6-(2,6-dimethylphenyl)pyrimidin-2-yl]-1-methyl-pyrazole-4-sulfonamide

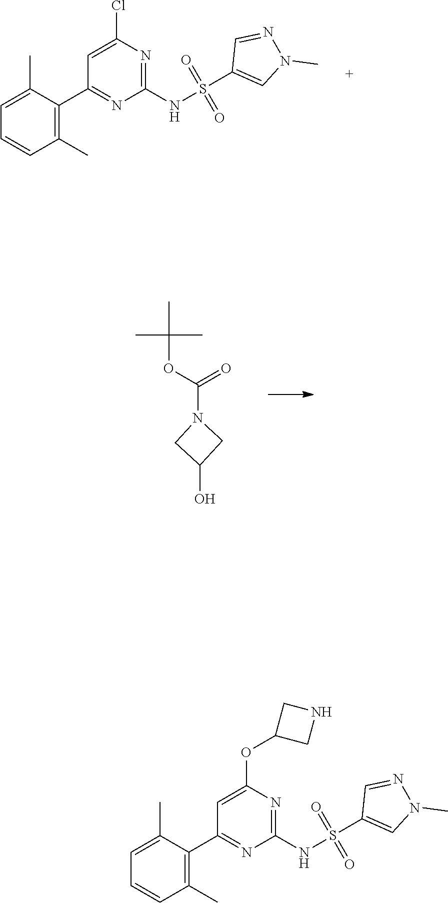

NaH (600 mg of 60% w/w, 15.00 mmol) was added to tert-butyl 3-hydroxyazetidine-1-carboxylate (1.78 g, 10.28 mmol) in NMP (10 mL) at 0° C. The mixture was stirred for 45 minutes. then added to N-[4-chloro-6-(2,6-dimethylphenyl)pyrimidin-2-yl]-1-methyl-pyrazole-4-sulfonamide (2.7 g, 7.146 mmol) in NMP (10 mL). The resulting mixture was stirred at 105° C. for 2 hours. The reaction mixture was cooled down to room temperature. The pH of the mixture was adjusted to ˜5 with 1 N HCl and extracted with ethyl acetate (3×20 mL). The combined organic layers were washed with brine, dried over Na₂SO₄, filtered, concentrated and purified on silica using a gradient of ethyl acetate and hexane. The product came out ˜50% ethyl acetate as a white foam tert-butyl 3-[6-(2,6-dimethylphenyl)-2-[(1-methylpyrazol-4-yl)sulfonylamino]pyrimidin-4-yl]oxyazetidine-1-carboxylate (3.71 g, 100%). ¹H NMR (400 MHz, Chloroform-d) δ 7.86 (d, J=5.5 Hz, 2H), 7.24 (d, J=7.6 Hz, 1H), 7.12 (d, J=7.6 Hz, 2H), 6.31 (s, 1H), 5.35 (s, 1H), 4.37-4.29 (m, 2H), 4.17 (d, J=6.6 Hz, 1H), 3.99 (d, J=11.6 Hz, 2H), 3.86 (s, 3H), 2.08 (s, 6H), 1.46 (s, 9H). ESI-MS m/z calc. 514.1998, found 515.0 (M+1)⁺; Retention time: 1.69 minutes (LC method A).

tert-Butyl 3-[6-(2,6-dimethylphenyl)-2-[(1-methylpyrazol-4-yl)sulfonylamino]pyrimidin-4-yl]oxyazetidine-1-carboxylate (3.71 g, 100%) was stirred in TFA (15 mL, 194.7 mmol)/DCM (15 mL) at room temperature for 30 minutes. The reaction mixture was concentrated to remove solvent. The crude was redissolved in DCM/toluene, concentrated (repeated 3 times) and used as was in the next step without further purification. N-[4-(azetidin-3-yloxy)-6-(2,6-dimethylphenyl)pyrimidin-2-yl]-1-methyl-pyrazole-4-sulfonamide (3.09 g, 99%).

¹H NMR (400 MHz, DMSO-d₆) δ 8.95 (s, 1H), 8.77 (s, 1H), 8.28 (s, 1H), 7.82 (s, 1H), 7.26 (dd, J=8.1, 7.0 Hz, 1H), 7.21-7.08 (m, 2H), 6.56 (s, 1H), 5.43-5.26 (m, 1H), 4.45-4.38 (m, 2H), 4.15-4.12 (m, 2H), 3.87 (s, 3H), 2.02 (s, 6H). ESI-MS m/z calc. 414.1474, found 416.0 (M+1)⁺; Retention time: 0.82 minutes (LC method A).

### Step 3: N-[4-(2,6-Dimethylphenyl)-6-[1-[(2S)-2-(dipropylamino)propanoyl]azetidin-3-yl]oxy-pyrimidin-2-yl]-1-methyl-pyrazole-4-sulfonamide

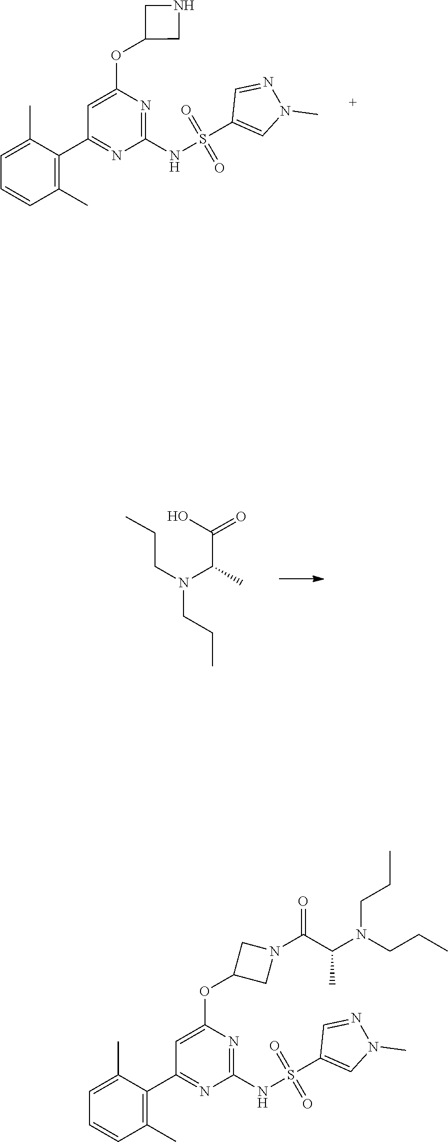

N-[4-(azetidin-3-yloxy)-6-(2,6-dimethylphenyl)pyrimidin-2-yl]-1-methyl-pyrazole-4-sulfonamide (30 mg, 0.07238 mmol) in DMF (0.2 mL) was added to (2S)-2-(dipropylamino)propanoic acid (approximately 12.54 mg, 0.07238 mmol) followed by HATU (in DMF (0.2 mL)), and K₂CO₃ (approximately 40.01 mg, 0.2895 mmol). The reaction mixture was shaken for 16 hours at 70° C. The reaction mixture was filtered and purified by reverse phase HPLC. N-[4-(2,6-dimethylphenyl)-6-[1-[(2S)-2-(dipropylamino)propanoyl]azetidin-3-yl]oxy-pyrimidin-2-yl]-1-methyl-pyrazole-4-sulfonamide (8.9 mg, 21%). ESI-MS m/z calc. 569.27844, found 570.29 (M+1)⁺; Retention time: 1.22 minutes (LC method A). ¹H NMR (400 MHz, DMSO-d₆) δ 9.69 (d, J=73.0 Hz, 1H), 8.33 (d, J=6.8 Hz, 1H), 7.82 (d, J=4.8 Hz, 1H), 7.30-7.21 (m, 1H), 7.14 (d, J=7.6 Hz, 2H), 6.54 (d, J=8.1 Hz, 1H), 5.37 (s, 1H), 4.65 (s, 1H), 4.49-4.29 (m, 2H), 4.11 (s, 1H), 4.00 (dd, J=29.7, 11.4 Hz, 2H), 3.87 (s, 3H), 3.04 (d, J=57.8 Hz, 4H), 2.01 (s, 6H), 1.66 (s, 4H), 1.44 (dd, J=19.3, 6.9 Hz, 3H), 0.96-0.85 (m, 6H).

### Example 245: Preparation of Compound 597

### Step 1: N-[4-[1-[1-(4-chlorophenyl)cyclopropanecarbonyl]azetidin-3-yl]oxy-6-(2,6-dimethylphenyl)pyrimidin-2-yl]-1-methyl-pyrazole-4-sulfonamide

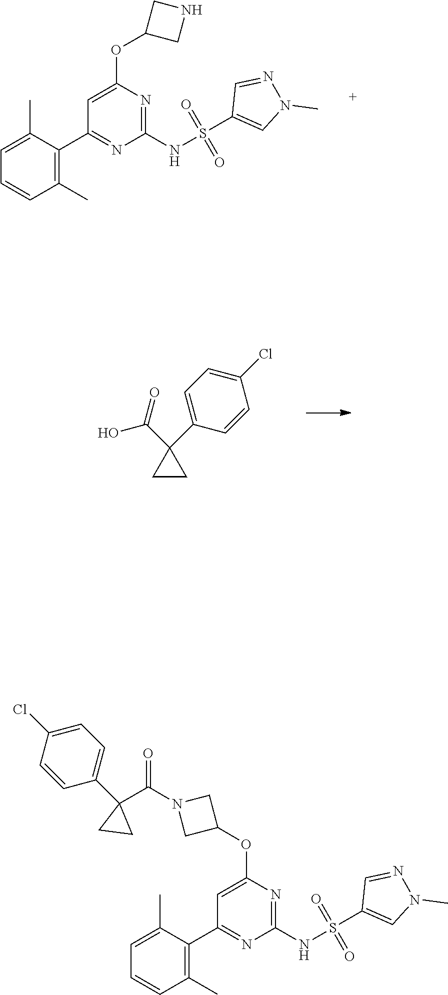 [SMILES: Cc1cccc(C)c1-c1cc(OC2CNC2)nc(NS(=O)(=O)c2cnn(C)c2)n1 ; O=C(O)C1(c2ccc(Cl)cc2)CC1 ; Cc1cccc(C)c1-c1cc(OC2CN(C(=O)C3(c4ccc(Cl)cc4)CC3)C2)nc(NS(=O)(=O)c2cnn(C)c2)n1]

N-[4-(azetidin-3-yloxy)-6-(2,6-dimethylphenyl)pyrimidin-2-yl]-1-methyl-pyrazole-4-sulfonamide (30 mg, 0.07238 mmol) in DMF (0.2 mL) was added to 1-(4-chlorophenyl)cyclopropanecarboxylic acid (approximately 21.35 mg, 0.1086 mmol), followed by HATU (approximately 41.29 mg, 0.1086 mmol) in DMF (0.2 mL), and K₂CO₃ (approximately 40.01 mg, 0.2895 mmol). The reaction mixture was shaken for 16 hours at 70° C. The reaction mixture was filtered and purified by reverse phase HPLC to give N-[4-[1-[1-(4-chlorophenyl)cyclopropanecarbonyl]azetidin-3-yl]oxy-6-(2,6-dimethylphenyl)pyrimidin-2-yl]-1-methyl-pyrazole-4-sulfonamide (17 mg, 40%). ESI-MS m/z calc. 592.16595, found 593.3 (M+1)⁺; Retention time: 1.74 minutes; LC method A. ¹H NMR (400 MHz, DMSO-d₆) δ 8.24 (s, 1H), 7.72 (s, 1H), 7.42-7.33 (m, 4H), 7.27-7.21 (m, 1H), 7.12 (d, J=7.6 Hz, 2H), 6.45 (s, 1H), 5.17 (s, 1H), 4.29 (s, 1H), 3.99 (s, 1H), 3.84 (s, 4H), 2.08 (s, 3H), 1.98 (s, 6H), 1.37 (s, 2H), 1.07 (d, J=28.3 Hz, 2H).

### Example 246: Preparation of Compound 598

### Step 1: N-[4-(2,6-dimethylphenyl)-6-[1-(1-phenylcyclobutanecarbonyl)azetidin-3-yl]oxy-pyrimidin-2-yl]-1-methyl-pyrazole-4-sulfonamide

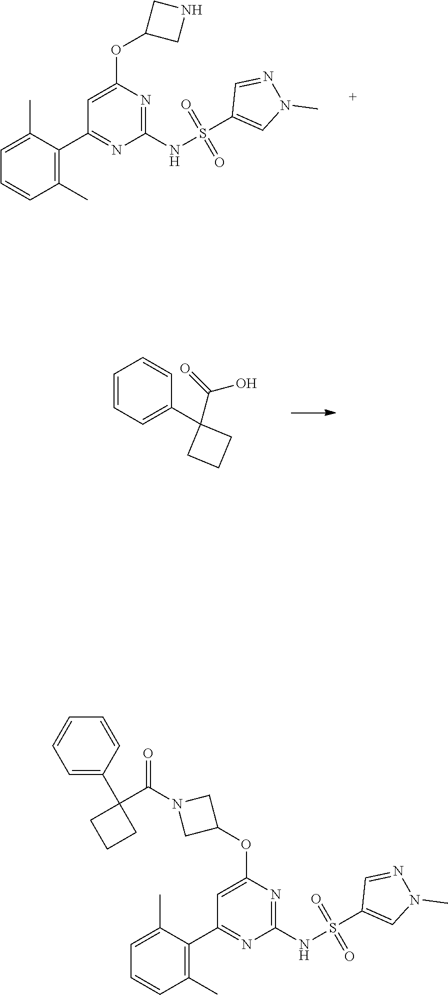 [SMILES: Cc1cccc(C)c1-c1cc(OC2CNC2)nc(NS(=O)(=O)c2cnn(C)c2)n1 ; O=C(O)C1(c2ccccc2)CCC1 ; Cc1cccc(C)c1-c1cc(OC2CN(C(=O)C3(c4ccccc4)CCC3)C2)nc(NS(=O)(=O)c2cnn(C)c2)n1]

N-[4-(azetidin-3-yloxy)-6-(2,6-dimethylphenyl)pyrimidin-2-yl]-1-methyl-pyrazole-4-sulfonamide (30 mg, 0.07238 mmol) in DMF (0.2 mL) was added to 1-phenylcyclobutanecarboxylic acid (approximately 19.14 mg, 0.1086 mmol), followed by HATU (approximately 41.29 mg, 0.1086 mmol) in DMF (0.2 mL), and K₂CO₃ (approximately 40.01 mg, 0.2895 mmol). The reaction mixture was shaken for 16 hours at 70° C. The reaction mixture was filtered and purified by reverse phase HPLC to give N-[4-(2,6-dimethylphenyl)-6-[1-(1-phenylcyclobutanecarbonyl)azetidin-3-yl]oxy-pyrimidin-2-yl]-1-methyl-pyrazole-4-sulfonamide (17.3 mg, 42%). ESI-MS m/z calc. 572.2206, found 573.35 (M+1)⁺; Retention time: 1.68 minutes; LC method A. ¹H NMR (400 MHz, DMSO-d₆) δ 11.60 (s, 1H), 8.22 (s, 1H), 7.74-7.66 (m, 1H), 7.43-7.39 (m, 1H), 7.39-7.33 (m, 3H), 7.29-7.24 (m, 1H), 7.24-7.20 (m, 1H), 7.16-7.07 (m, 2H), 6.44 (s, 1H), 5.23-5.10 (m, 1H), 4.30-4.15 (m, 2H), 3.83 (s, 3H), 3.82-3.77 (m, 1H), 3.68-3.64 (m, 1H), 2.79-2.67 (m, 2H), 2.41-2.24 (m, 2H), 1.98 (s, 6H), 1.87-1.78 (m, 2H).

### Example 247: Preparation of Compound 599

### Step 1: N-[4-[1-[1-(4-chlorophenyl)cyclohexanecarbonyl]azetidin-3-yl]oxy-6-(2,6-dimethylphenyl)pyrimidin-2-yl]-1-methyl-pyrazole-4-sulfonamide

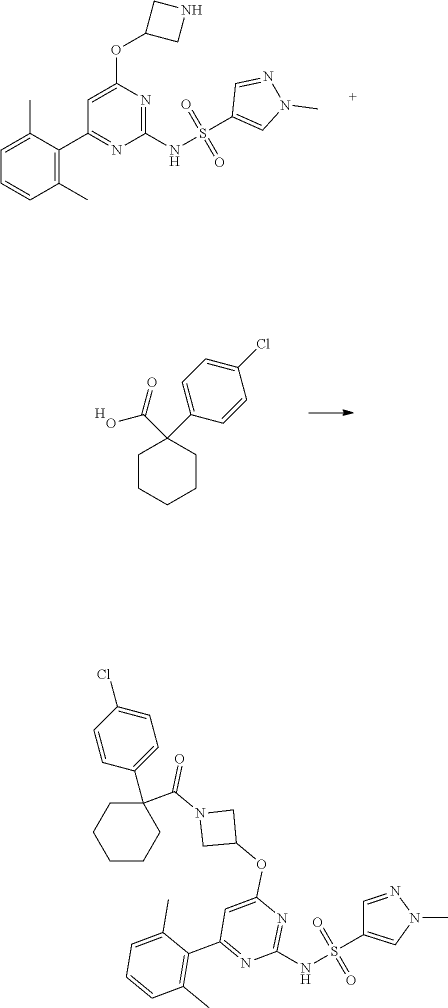

N-[4-(azetidin-3-yloxy)-6-(2,6-dimethylphenyl)pyrimidin-2-yl]-1-methyl-pyrazole-4-sulfonamide (30 mg, 0.07238 mmol) in DMF (0.2 mL) was added to 1-(4-chlorophenyl)cyclohexanecarboxylic acid (approximately 25.92 mg, 0.1086 mmol) followed by HATU (approximately 41.29 mg, 0.1086 mmol) in DMF (0.2 mL), and K₂CO₃ (approximately 40.01 mg, 0.2895 mmol). The reaction mixture was shaken for 16 hours at 70° C. The reaction mixture was filtered and purified by reverse phase HPLC. N-[4-[1-[1-(4-chlorophenyl)cyclohexanecarbonyl]azetidin-3-yl]oxy-6-(2,6-dimethylphenyl)pyrimidin-2-yl]-1 -methyl-pyrazole-4-sulfonamide (12.7 mg, 28%). ESI-MS m/z calc. 634.2129, found 635.35 (M+1)⁺; Retention time: 1.95 minutes; LC method A. ¹H NMR (400 MHz, DMSO-d₆) δ 11.58 (s, 1H), 8.18 (s, 1H), 7.66 (s, 1H), 7.46-7.40 (m, 2H), 7.37-7.31 (m, 2H), 7.26-7.20 (m, 1H), 7.16-7.08 (m, 2H), 6.45 (s, 1H), 5.17-4.97 (m, 1H), 4.25 (s, 1H), 4.10 (s, 1H), 3.83 (s, 3H), 3.65 (s, 1H), 2.24-2.13 (m, 2H), 2.08 (s, 3H), 1.98 (s, 6H), 1.57 (s, 5H), 1.32-1.22 (m, 1H).

### Example 248: Preparation of Compound 600

### Step 1: N-[4-[1-(2,1-benzoxazole-3-carbonyl)azetidin-3-yl]oxy-6-(2,6-dimethylphenyl)pyrimidin-2-yl]-1-methyl-pyrazole-4-sulfonamide

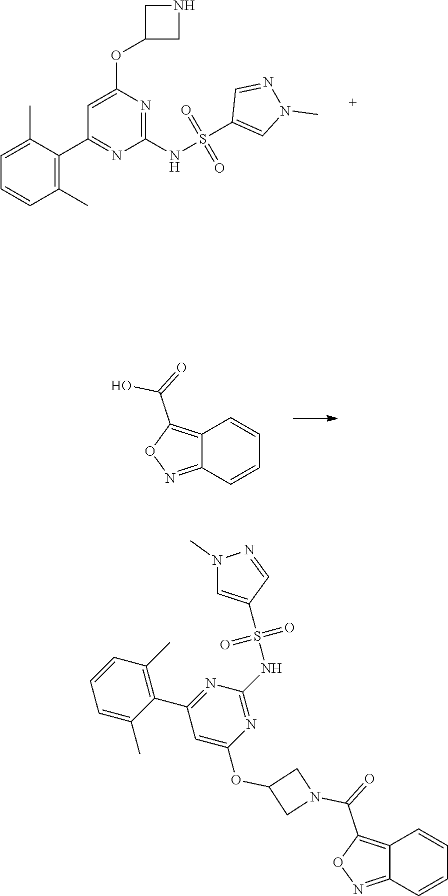 [SMILES: Cc1cccc(C)c1-c1cc(OC2CNC2)nc(NS(=O)(=O)c2cnn(C)c2)n1 ; O=C(O)c1onc2ccccc12 ; Cc1cccc(C)c1-c1cc(OC2CN(C(=O)c3onc4ccccc34)C2)nc(NS(=O)(=O)c2cnn(C)c2)n1]

N-[4-(azetidin-3-yloxy)-6-(2,6-dimethylphenyl)pyrimidin-2-yl]-1-methyl-pyrazole-4-sulfonamide (30 mg, 0.07238 mmol) in DMF (0.2 mL) was added to 2,1-benzoxazole-3-carboxylic acid (approximately 11.81 mg, 0.07238 mmol), followed by HATU (in DMF (0.2 mL)), and K₂CO₃. The reaction mixture was shaken for 16 hours at 70° C. The reaction mixture was filtered and purified by reverse phase HPLC to give N-[4-[1-(2,1-benzoxazole-3-carbonyl)azetidin-3-yl] oxy-6-(2,6-dimethylphenyl)pyrimidin-2-yl]-1-methyl-pyrazole-4-sulfonamide (16.8 mg, 42% ). ESI-MS m/z calc. 559.16376, found 560.18 (M+1)⁺; Retention time: 1.63 minutes; LC method A. ¹H NMR (400 MHz, DMSO-d₆) δ 11.71 (s, 1H), 8.35 (s, 1H), 7.98 (dt, J=8.8, 1.1 Hz, 1H), 7.80 (d, J=9.0 Hz, 2H), 7.56-7.50 (m, 1H), 7.34-7.28 (m, 1H), 7.24 (s, 1H), 7.14 (d, J=7.6 Hz, 2H), 6.58 (d, J=13.3 Hz, 1H), 5.46 (d, J=3.5 Hz, 1H), 5.16 (dd, J=10.8, 6.7 Hz, 1H), 4.72-4.60 (m, 2H), 4.20 (d, J=10.4 Hz, 1H), 3.87 (s, 3H), 2.00 (s, 6H).

### Example 249: Characterization of Compounds 601-699

The compounds in the following tables were prepared in a manner analogous to that described above using commercially available reagents and intermediates described herein.

| | | LCMS | | | |
|---|---|---|---|---|---|
| | | Room | | | |
| Compound | | temperature | Calc. | | LCMS |
| number | Structure | (min) | mass | M + 1 | Method |
| 601 | 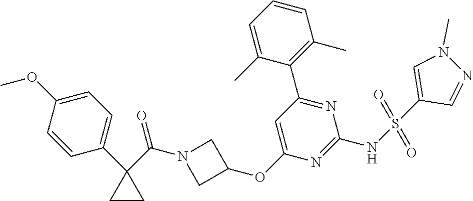 [SMILES: COc1ccc(C2(C(=O)N3CC(Oc4cc(-c5c(C)cccc5C)nc(NS(=O)(=O)c5cnn(C)c5)n4)C3)CC2)cc1] | 1.63 | 588.215 | 589.35 | A |
| 602 | 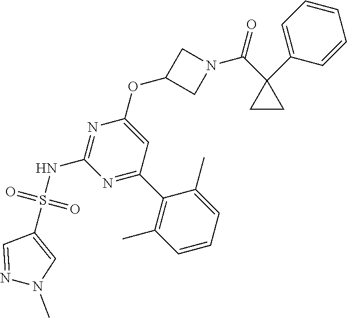 [SMILES: Cc1cccc(C)c1-c1cc(OC2CN(C(=O)C3(c4ccccc4)CC3)C2)nc(NS(=O)(=O)c2cnn(C)c2)n1] | 1.65 | 558.205 | 559.3 | A |
| 603 | 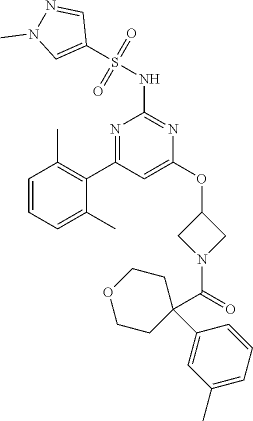 [SMILES: Cc1cccc(C2(C(=O)N3CC(Oc4cc(-c5c(C)cccc5C)nc(NS(=O)(=O)c5cnn(C)c5)n4)C3)CCOCC2)c1] | 1.62 | 616.247 | 617.36 | A |
| 604 | 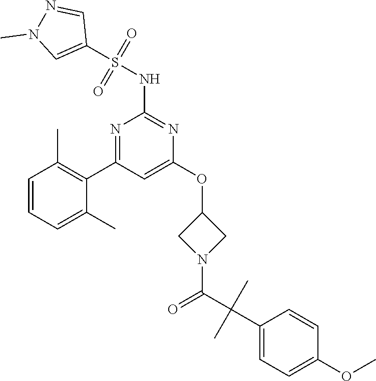 | 1.6 | 590.231 | 591.34 | A |
| 605 | 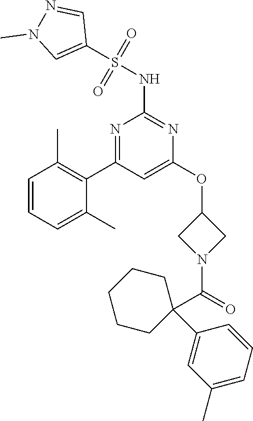 [SMILES: Cc1cccc(C2(C(=O)N3CC(Oc4cc(-c5c(C)cccc5C)nc(NS(=O)(=O)c5cnn(C)c5)n4)C3)CCCCC2)c1] | 1.92 | 614.268 | 615.4 | A |
| 606 | 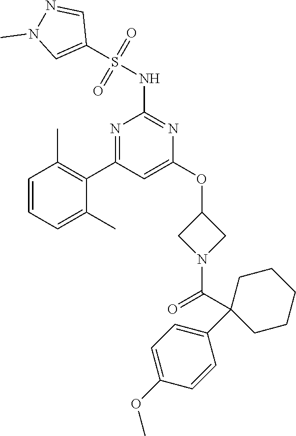 [SMILES: COc1ccc(C2(C(=O)N3CC(Oc4cc(-c5c(C)cccc5C)nc(NS(=O)(=O)c5cnn(C)c5)n4)C3)CCCCC2)cc1] | 1.82 | 630.262 | 631.4 | A |
| 607 | 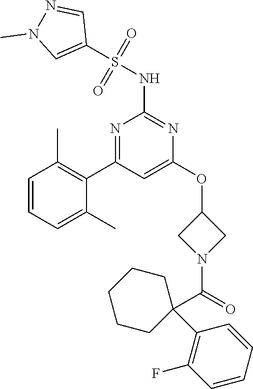 [SMILES: Cc1cccc(C)c1-c1cc(OC2CN(C(=O)C3(c4ccccc4F)CCCCC3)C2)nc(NS(=O)(=O)c2cnn(C)c2)n1] | 1.82 | 618.242 | 619.39 | A |
| 608 | 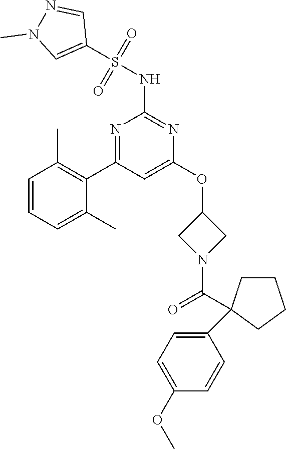 [SMILES: COc1ccc(C2(C(=O)N3CC(Oc4cc(-c5c(C)cccc5C)nc(NS(=O)(=O)c5cnn(C)c5)n4)C3)CCCC2)cc1] | 1.75 | 616.247 | 617.36 | A |
| 609 | 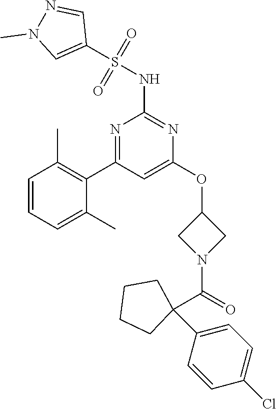 [SMILES: Cc1cccc(C)c1-c1cc(OC2CN(C(=O)C3(c4ccc(Cl)cc4)CCCC3)C2)nc(NS(=O)(=O)c2cnn(C)c2)n1] | 1.87 | 620.197 | 621.35 | A |
| 610 | 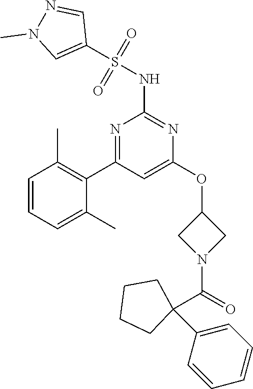 [SMILES: Cc1cccc(C)c1-c1cc(OC2CN(C(=O)C3(c4ccccc4)CCCC3)C2)nc(NS(=O)(=O)c2cnn(C)c2)n1] | 1.77 | 586.236 | 587.35 | A |
| 611 | 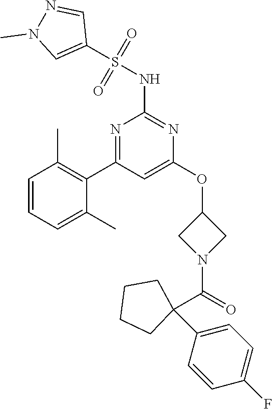 [SMILES: Cc1cccc(C)c1-c1cc(OC2CN(C(=O)C3(c4ccc(F)cc4)CCCC3)C2)nc(NS(=O)(=O)c2cnn(C)c2)n1] | 1.78 | 604.227 | 605.35 | A |
| 612 | 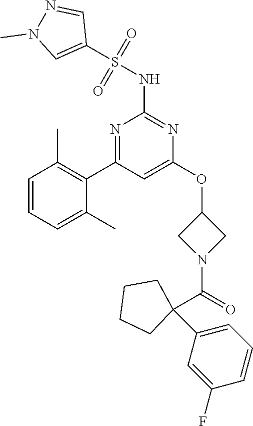 [SMILES: Cc1cccc(C)c1-c1cc(OC2CN(C(=O)C3(c4cccc(F)c4)CCCC3)C2)nc(NS(=O)(=O)c2cnn(C)c2)n1] | 1.78 | 604.227 | 605.35 | A |
| 613 | 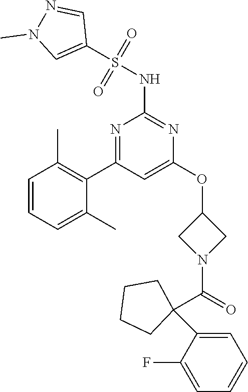 [SMILES: Cc1cccc(C)c1-c1cc(OC2CN(C(=O)C3(c4ccccc4F)CCCC3)C2)nc(NS(=O)(=O)c2cnn(C)c2)n1] | 1.73 | 604.227 | 605.35 | A |
| 614 | 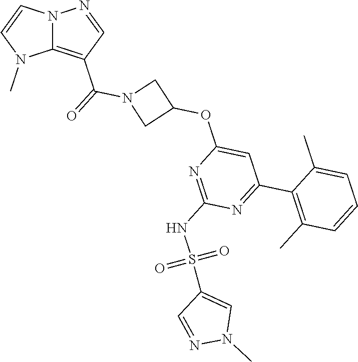 [SMILES: Cc1cccc(C)c1-c1cc(OC2CN(C(=O)c3cnn4ccn(C)c34)C2)nc(NS(=O)(=O)c2cnn(C)c2)n1] | 1.33 | 561.191 | 562.16 | A |
| 615 | 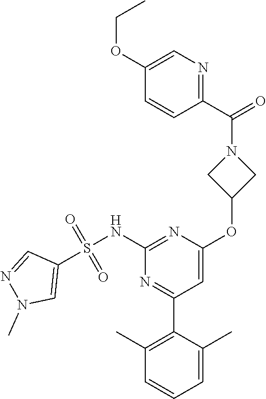 [SMILES: CCOc1ccc(C(=O)N2CC(Oc3cc(-c4c(C)cccc4C)nc(NS(=O)(=O)c4cnn(C)c4)n3)C2)nc1] | 1.6 | 563.195 | 564.21 | A |
| 616 | 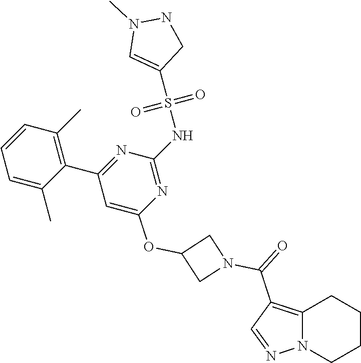 [SMILES: Cc1cccc(C)c1-c1cc(OC2CN(C(=O)c3cnn4c3CCCC4)C2)nc(NS(=O)(=O)C2=CN(C)NC2)n1] | 1.33 | 562.211 | 563.22 | A |
| 617 | 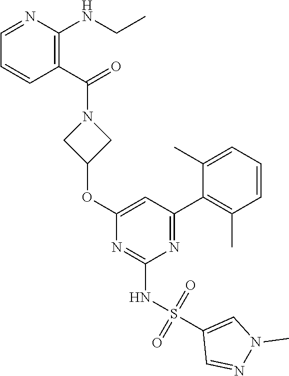 [SMILES: CCNc1ncccc1C(=O)N1CC(Oc2cc(-c3c(C)cccc3C)nc(NS(=O)(=O)c3cnn(C)c3)n2)C1] | 1.16 | 562.211 | 563.22 | A |
| 618 | 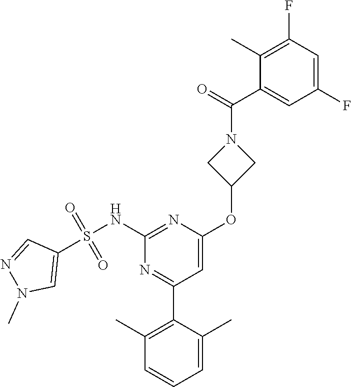 [SMILES: Cc1cccc(C)c1-c1cc(OC2CN(C(=O)c3cc(F)cc(F)c3C)C2)nc(NS(=O)(=O)c2cnn(C)c2)n1] | 1.63 | 568.17 | 569.17 | A |
| 619 | 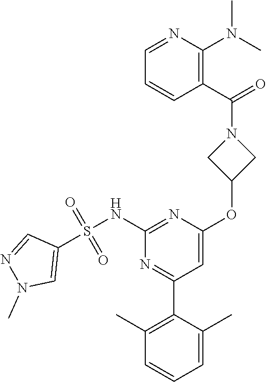 [SMILES: Cc1cccc(C)c1-c1cc(OC2CN(C(=O)c3cccnc3N(C)C)C2)nc(NS(=O)(=O)c2cnn(C)c2)n1] | 1.08 | 562.211 | 563.22 | A |
| 620 | 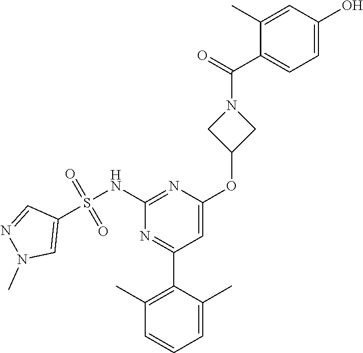 [SMILES: Cc1cc(O)ccc1C(=O)N1CC(Oc2cc(-c3c(C)cccc3C)nc(NS(=O)(=O)c3cnn(C)c3)n2)C1] | 1.33 | 548.184 | 549.14 | A |
| 621 | 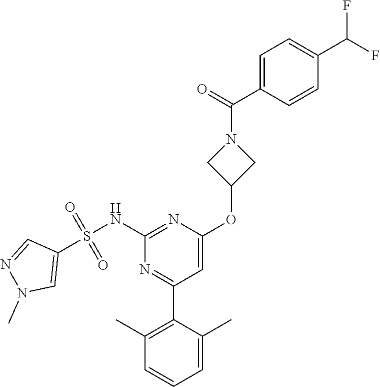 [SMILES: Cc1cccc(C)c1-c1cc(OC2CN(C(=O)c3ccc(C(F)F)cc3)C2)nc(NS(=O)(=O)c2cnn(C)c2)n1] | 1.59 | 568.17 | 569.17 | A |
| 622 | 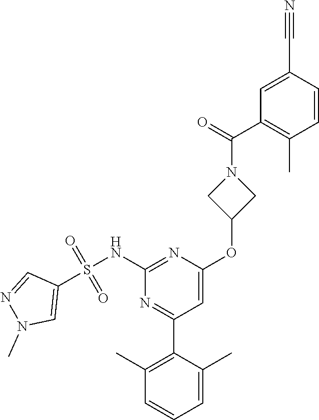 [SMILES: Cc1ccc(C#N)cc1C(=O)N1CC(Oc2cc(-c3c(C)cccc3C)nc(NS(=O)(=O)c3cnn(C)c3)n2)C1] | 1.5 | 557.185 | 558.2 | A |
| 623 | 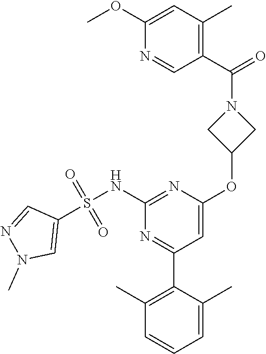 [SMILES: COc1cc(C)c(C(=O)N2CC(Oc3cc(-c4c(C)cccc4C)nc(NS(=O)(=O)c4cnn(C)c4)n3)C2)cn1] | 1.41 | 563.195 | 564.21 | A |
| 624 | 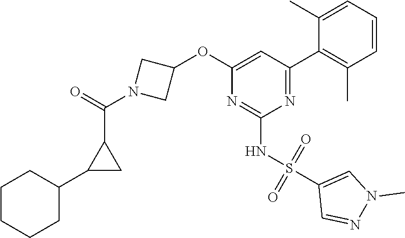 [SMILES: Cc1cccc(C)c1-c1cc(OC2CN(C(=O)C3CC3C3CCCCC3)C2)nc(NS(=O)(=O)c2cnn(C)c2)n1] | 1.86 | 564.252 | 565.27 | A |
| 625 | 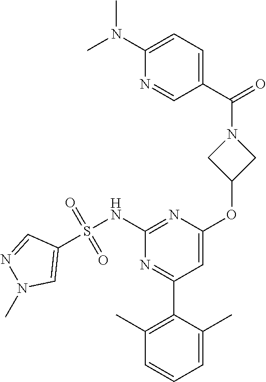 [SMILES: Cc1cccc(C)c1-c1cc(OC2CN(C(=O)c3ccc(N(C)C)nc3)C2)nc(NS(=O)(=O)c2cnn(C)c2)n1] | 1.09 | 562.211 | 563.22 | A |
| 626 | 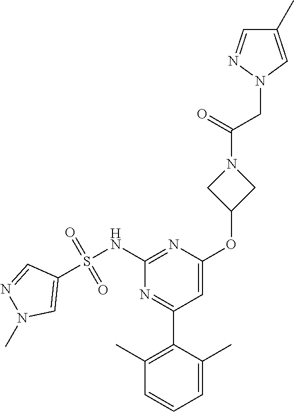 [SMILES: Cc1cnn(CC(=O)N2CC(Oc3cc(-c4c(C)cccc4C)nc(NS(=O)(=O)c4cnn(C)c4)n3)C2)c1] | 1.33 | 536.195 | 537.18 | A |
| 627 | 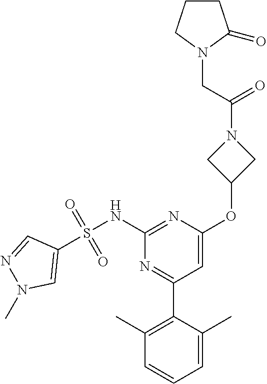 [SMILES: Cc1cccc(C)c1-c1cc(OC2CN(C(=O)CN3CCCC3=O)C2)nc(NS(=O)(=O)c2cnn(C)c2)n1] | 1.18 | 539.195 | 540.22 | A |
| 628 | 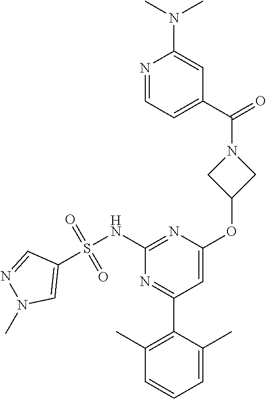 [SMILES: Cc1cccc(C)c1-c1cc(OC2CN(C(=O)c3ccnc(N(C)C)c3)C2)nc(NS(=O)(=O)c2cnn(C)c2)n1] | 1.11 | 562.211 | 563.22 | A |
| 629 | 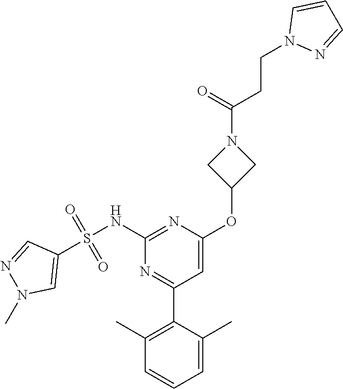 [SMILES: Cc1cccc(C)c1-c1cc(OC2CN(C(=O)CCn3cccn3)C2)nc(NS(=O)(=O)c2cnn(C)c2)n1] | 1.26 | 536.195 | 537.18 | A |
| 630 | 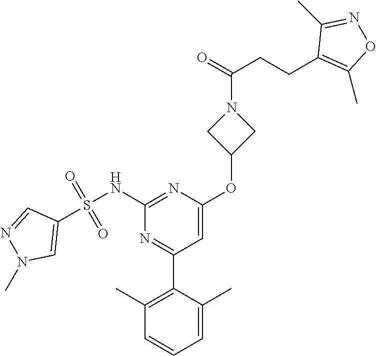 [SMILES: Cc1cccc(C)c1-c1cc(OC2CN(C(=O)CCc3c(C)noc3C)C2)nc(NS(=O)(=O)c2cnn(C)c2)n1] | 1.37 | 565.211 | 566.2 | A |
| 631 | 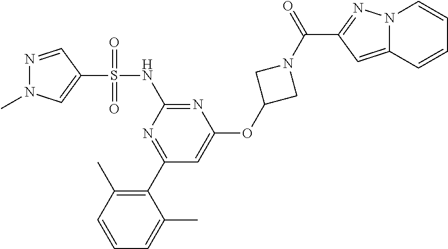 [SMILES: Cc1cccc(C)c1-c1cc(OC2CN(C(=O)c3cc4ccccn4n3)C2)nc(NS(=O)(=O)c2cnn(C)c2)n1] | 1.51 | 558.18 | 559.19 | A |
| 632 | 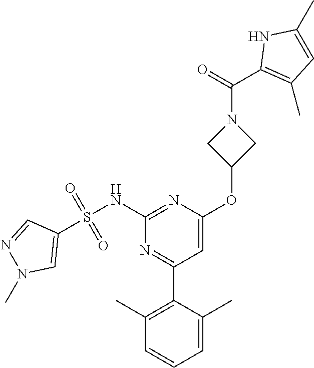 [SMILES: Cc1cc(C)c(C(=O)N2CC(Oc3cc(-c4c(C)cccc4C)nc(NS(=O)(=O)c4cnn(C)c4)n3)C2)[nH]1] | 1.5 | 535.2 | 536.19 | A |
| 633 | 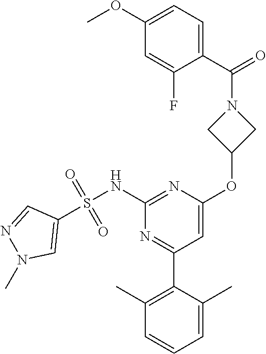 [SMILES: COc1ccc(C(=O)N2CC(Oc3cc(-c4c(C)cccc4C)nc(NS(=O)(=O)c4cnn(C)c4)n3)C2)c(F)c1] | 1.55 | 566.175 | 567.19 | A |
| 634 | 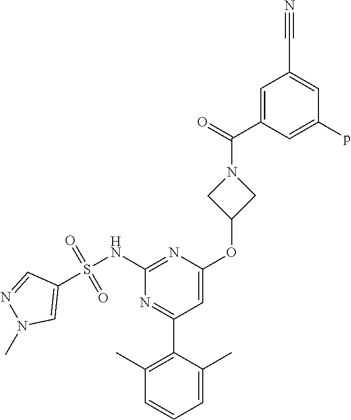 | 1.54 | 561.159 | 562.16 | A |
| 635 | 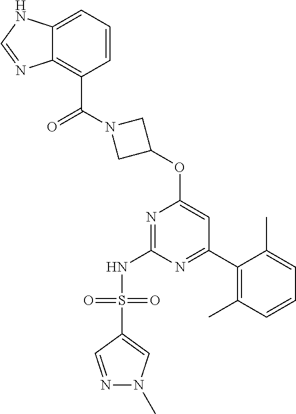 [SMILES: Cc1cccc(C)c1-c1cc(OC2CN(C(=O)c3cccc4[nH]cnc34)C2)nc(NS(=O)(=O)c2cnn(C)c2)n1] | 1.04 | 558.18 | 559 | Q |
| 636 | 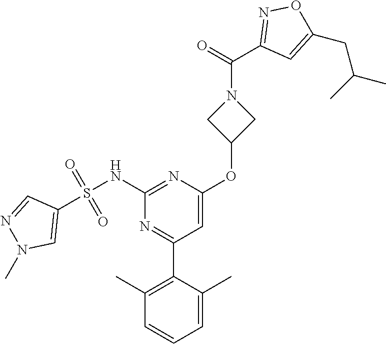 [SMILES: Cc1cccc(C)c1-c1cc(OC2CN(C(=O)c3cc(CC(C)C)on3)C2)nc(NS(=O)(=O)c2cnn(C)c2)n1] | 1.79 | 565.211 | 566.2 | A |
| 637 | 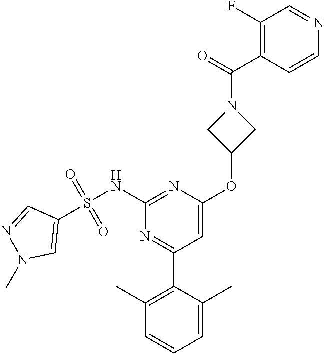 [SMILES: Cc1cccc(C)c1-c1cc(OC2CN(C(=O)c3ccncc3F)C2)nc(NS(=O)(=O)c2cnn(C)c2)n1] | 1.33 | 537.159 | 538.17 | A |
| 638 | 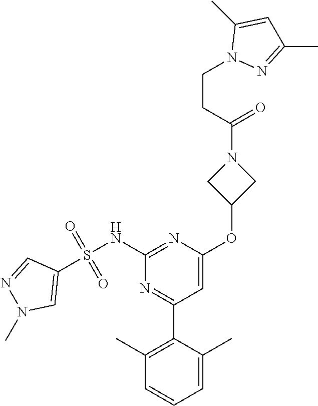 [SMILES: Cc1cc(C)n(CCC(=O)N2CC(Oc3cc(-c4c(C)cccc4C)nc(NS(=O)(=O)c4cnn(C)c4)n3)C2)n1] | 1.27 | 564.227 | 565.2 | A |
| 639 | 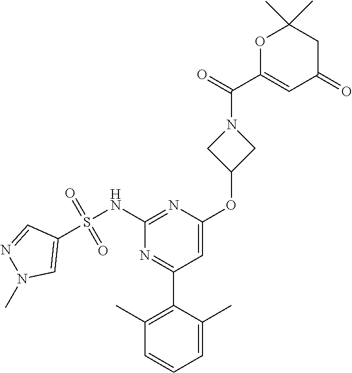 [SMILES: Cc1cccc(C)c1-c1cc(OC2CN(C(=O)C3=CC(=O)CC(C)(C)O3)C2)nc(NS(=O)(=O)c2cnn(C)c2)n1] | 1.43 | 566.195 | 567.19 | A |
| 640 | 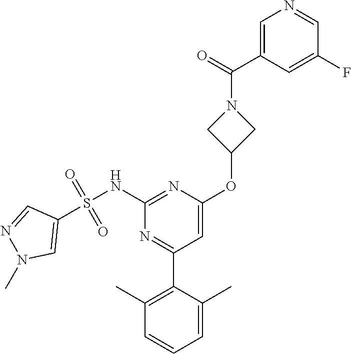 [SMILES: Cc1cccc(C)c1-c1cc(OC2CN(C(=O)c3cncc(F)c3)C2)nc(NS(=O)(=O)c2cnn(C)c2)n1] | 1.35 | 537.159 | 538.17 | A |
| 641 | 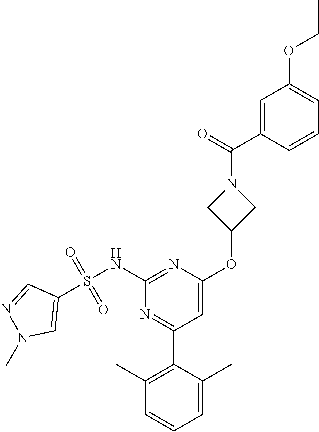 [SMILES: CCOc1cccc(C(=O)N2CC(Oc3cc(-c4c(C)cccc4C)nc(NS(=O)(=O)c4cnn(C)c4)n3)C2)c1] | 1.61 | 562.2 | 563.22 | A |
| 642 | 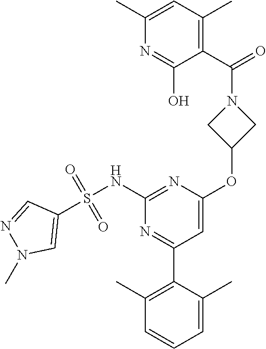 [SMILES: Cc1cc(C)c(C(=O)N2CC(Oc3cc(-c4c(C)cccc4C)nc(NS(=O)(=O)c4cnn(C)c4)n3)C2)c(O)n1] | 1.21 | 563.195 | 564.21 | A |
| 643 | 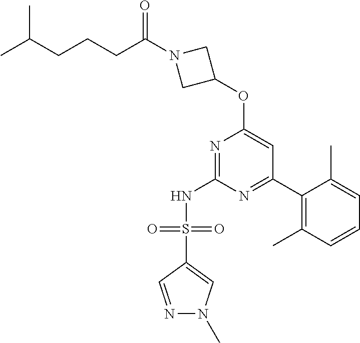 [SMILES: Cc1cccc(C)c1-c1cc(OC2CN(C(=O)CCCC(C)C)C2)nc(NS(=O)(=O)c2cnn(C)c2)n1] | 1.58 | 526.236 | 527.2 | A |
| 644 | 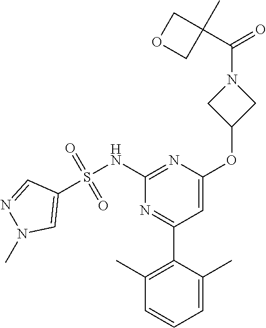 [SMILES: Cc1cccc(C)c1-c1cc(OC2CN(C(=O)C3(C)COC3)C2)nc(NS(=O)(=O)c2cnn(C)c2)n1] | 1.24 | 512.184 | 513.19 | A |
| 645 | 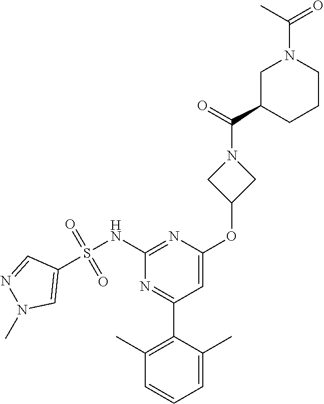 [SMILES: CC(=O)N1CCC[C@@H](C(=O)N2CC(Oc3cc(-c4c(C)cccc4C)nc(NS(=O)(=O)c4cnn(C)c4)n3)C2)C1] | 1.24 | 567.226 | 568.24 | A |
| 646 | 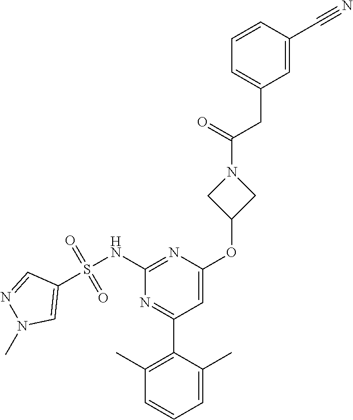 [SMILES: Cc1cccc(C)c1-c1cc(OC2CN(C(=O)Cc3cccc(C#N)c3)C2)nc(NS(=O)(=O)c2cnn(C)c2)n1] | 1.45 | 557.185 | 558.2 | A |
| 647 | 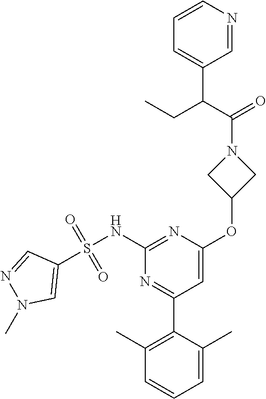 [SMILES: CCC(C(=O)N1CC(Oc2cc(-c3c(C)cccc3C)nc(NS(=O)(=O)c3cnn(C)c3)n2)C1)c1cccnc1] | 1.21 | 561.216 | 562.31 | A |
| 648 | 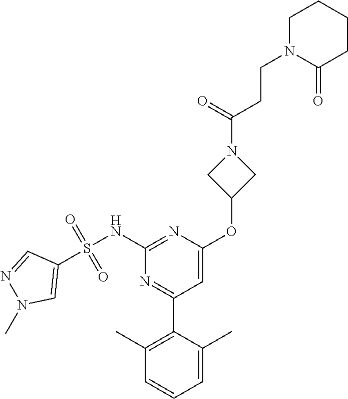 [SMILES: Cc1cccc(C)c1-c1cc(OC2CN(C(=O)CCN3CCCCC3=O)C2)nc(NS(=O)(=O)c2cnn(C)c2)n1] | 1.26 | 567.226 | 568.32 | A |
| 649 | 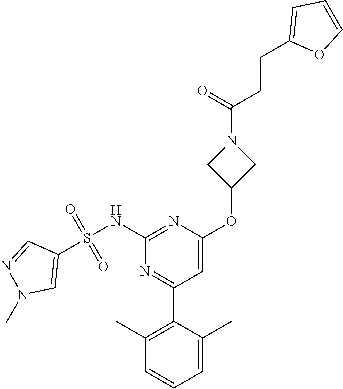 [SMILES: Cc1cccc(C)c1-c1cc(OC2CN(C(=O)CCc3ccco3)C2)nc(NS(=O)(=O)c2cnn(C)c2)n1] | 1.48 | 536.184 | 537.25 | A |
| 650 | 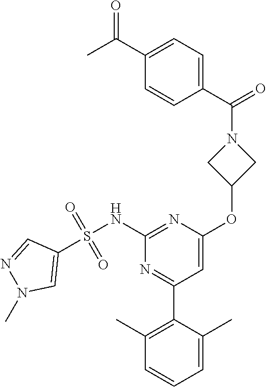 [SMILES: CC(=O)c1ccc(C(=O)N2CC(Oc3cc(-c4c(C)cccc4C)nc(NS(=O)(=O)c4cnn(C)c4)n3)C2)cc1] | 1.45 | 560.184 | 561.32 | A |
| 651 | 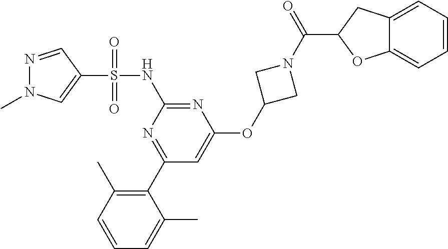 [SMILES: Cc1cccc(C)c1-c1cc(OC2CN(C(=O)C3Cc4ccccc4O3)C2)nc(NS(=O)(=O)c2cnn(C)c2)n1] | 1.6 | 560.184 | 561.25 | A |
| 652 | 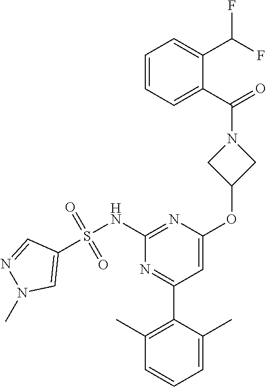 [SMILES: Cc1cccc(C)c1-c1cc(OC2CN(C(=O)c3ccccc3C(F)F)C2)nc(NS(=O)(=O)c2cnn(C)c2)n1] | 1.6 | 568.17 | 569.25 | A |
| 653 | 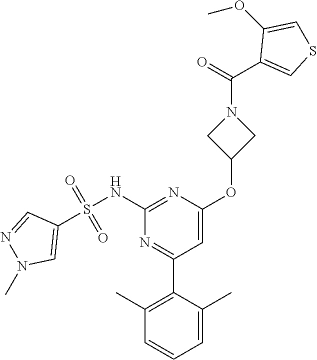 [SMILES: COc1cscc1C(=O)N1CC(Oc2cc(-c3c(C)cccc3C)nc(NS(=O)(=O)c3cnn(C)c3)n2)C1] | 1.45 | 554.141 | 555.23 | A |
| 654 | 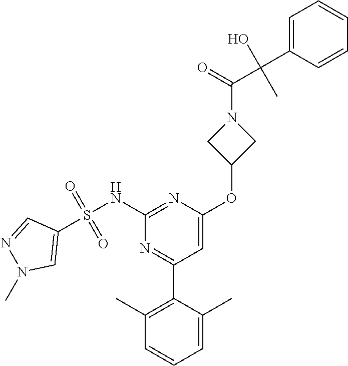 [SMILES: Cc1cccc(C)c1-c1cc(OC2CN(C(=O)C(C)(O)c3ccccc3)C2)nc(NS(=O)(=O)c2cnn(C)c2)n1] | 1.47 | 562.2 | 563.3 | A |
| 655 | 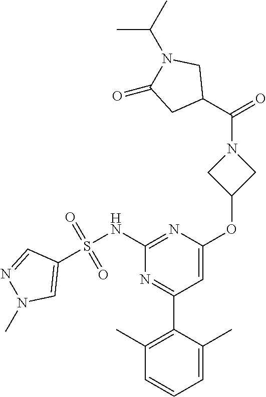 [SMILES: Cc1cccc(C)c1-c1cc(OC2CN(C(=O)C3CC(=O)N(C(C)C)C3)C2)nc(NS(=O)(=O)c2cnn(C)c2)n1] | 1.3 | 567.226 | 568.32 | A |
| 656 | 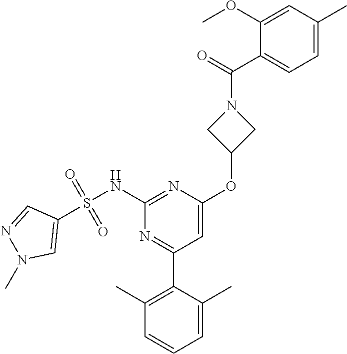 [SMILES: COc1cc(C)ccc1C(=O)N1CC(Oc2cc(-c3c(C)cccc3C)nc(NS(=O)(=O)c3cnn(C)c3)n2)C1] | 1.55 | 562.2 | 563.3 | A |
| 657 | 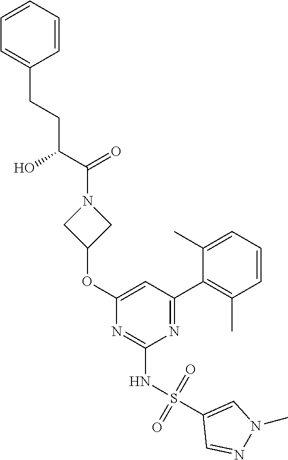 [SMILES: Cc1cccc(C)c1-c1cc(OC2CN(C(=O)[C@H](O)CCc3ccccc3)C2)nc(NS(=O)(=O)c2cnn(C)c2)n1] | 1.53 | 576.215 | 577.32 | A |
| 658 | 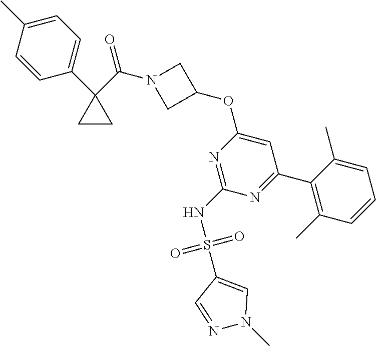 [SMILES: Cc1ccc(C2(C(=O)N3CC(Oc4cc(-c5c(C)cccc5C)nc(NS(=O)(=O)c5cnn(C)c5)n4)C3)CC2)cc1] | 1.75 | 572.221 | 573.28 | A |
| 659 | 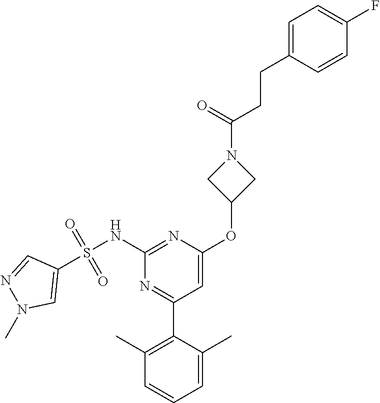 [SMILES: Cc1cccc(C)c1-c1cc(OC2CN(C(=O)CCc3ccc(F)cc3)C2)nc(NS(=O)(=O)c2cnn(C)c2)n1] | 1.6 | 564.196 | 565.28 | A |
| 660 | 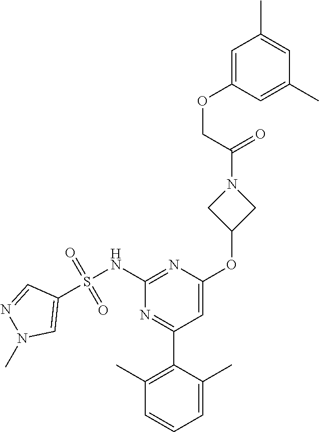 [SMILES: Cc1cc(C)cc(OCC(=O)N2CC(Oc3cc(-c4c(C)cccc4C)nc(NS(=O)(=O)c4cnn(C)c4)n3)C2)c1] | 1.74 | 576.215 | 577.32 | A |
| 661 | 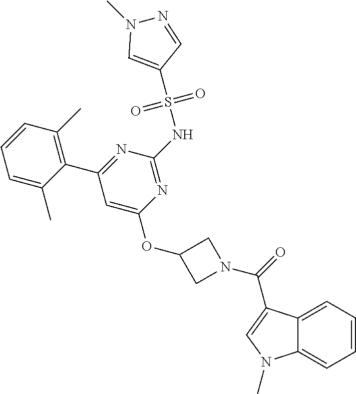 [SMILES: Cc1cccc(C)c1-c1cc(OC2CN(C(=O)c3cn(C)c4ccccc34)C2)nc(NS(=O)(=O)c2cnn(C)c2)n1] | 1.57 | 571.2 | 572.29 | A |
| 662 | 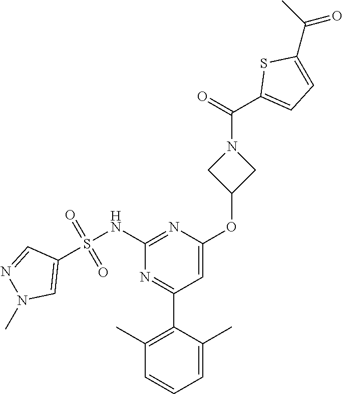 [SMILES: CC(=O)c1ccc(C(=O)N2CC(Oc3cc(-c4c(C)cccc4C)nc(NS(=O)(=O)c4cnn(C)c4)n3)C2)s1] | 1.48 | 566.141 | 567.27 | A |
| 663 | 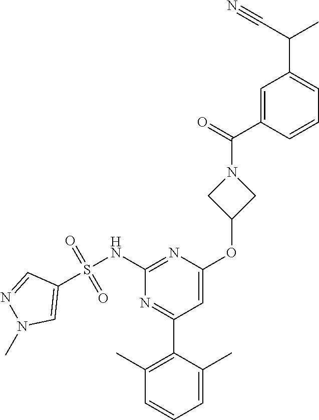 [SMILES: Cc1cccc(C)c1-c1cc(OC2CN(C(=O)c3cccc(C(C)C#N)c3)C2)nc(NS(=O)(=O)c2cnn(C)c2)n1] | 1.53 | 571.2 | 572.29 | A |
| 664 | 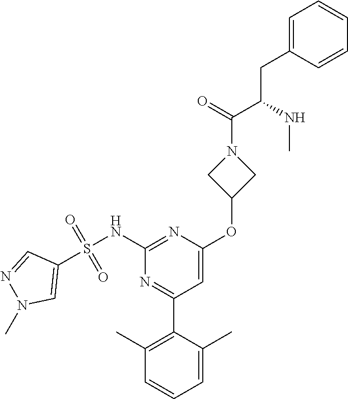 [SMILES: CN[C@@H](Cc1ccccc1)C(=O)N1CC(Oc2cc(-c3c(C)cccc3C)nc(NS(=O)(=O)c3cnn(C)c3)n2)C1] | 1.28 | 575.231 | 576.32 | A |
| 665 | 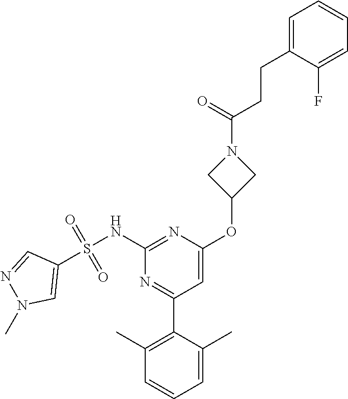 [SMILES: Cc1cccc(C)c1-c1cc(OC2CN(C(=O)CCc3ccccc3F)C2)nc(NS(=O)(=O)c2cnn(C)c2)n1] | 1.61 | 564.196 | 565.28 | A |
| 666 | 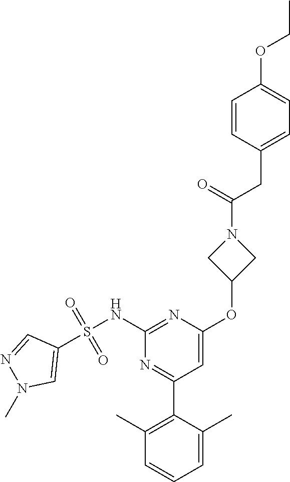 [SMILES: CCOc1ccc(CC(=O)N2CC(Oc3cc(-c4c(C)cccc4C)nc(NS(=O)(=O)c4cnn(C)c4)n3)C2)cc1] | 1.61 | 576.215 | 577.32 | A |
| 667 | 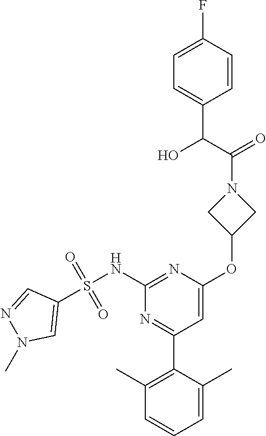 [SMILES: Cc1cccc(C)c1-c1cc(OC2CN(C(=O)C(O)c3ccc(F)cc3)C2)nc(NS(=O)(=O)c2cnn(C)c2)n1] | 1.44 | 566.175 | 567.27 | A |
| 668 | 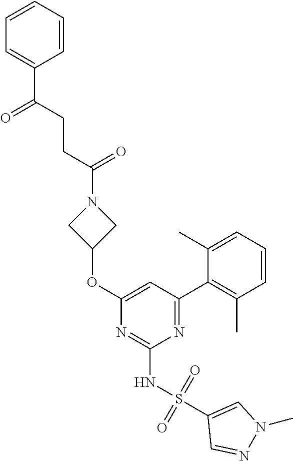 [SMILES: Cc1cccc(C)c1-c1cc(OC2CN(C(=O)CCC(=O)c3ccccc3)C2)nc(NS(=O)(=O)c2cnn(C)c2)n1] | 1.54 | 574.2 | 575.33 | A |
| 669 | 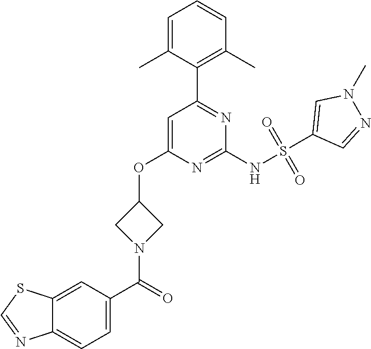 [SMILES: Cc1cccc(C)c1-c1cc(OC2CN(C(=O)c3ccc4ncsc4c3)C2)nc(NS(=O)(=O)c2cnn(C)c2)n1] | 1.03 | 575.141 | 576.34 | R |
| 670 | 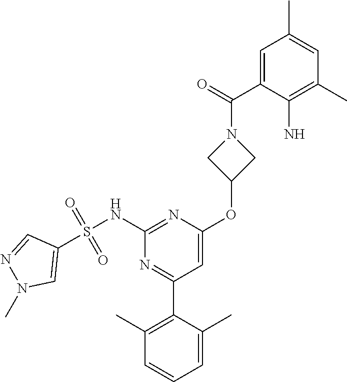 [SMILES: Cc1cc(C)c(N)c(C(=O)N2CC(Oc3cc(-c4c(C)cccc4C)nc(NS(=O)(=O)c4cnn(C)c4)n3)C2)c1] | 1.12 | 561.216 | 562.37 | R |
| 671 | 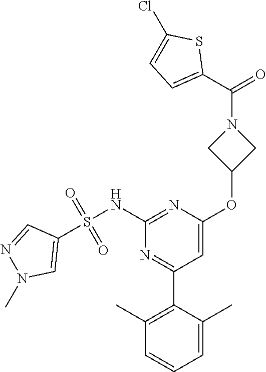 [SMILES: Cc1cccc(C)c1-c1cc(OC2CN(C(=O)c3ccc(Cl)s3)C2)nc(NS(=O)(=O)c2cnn(C)c2)n1] | 1.22 | 558.091 | 559.26 | R |
| 672 | 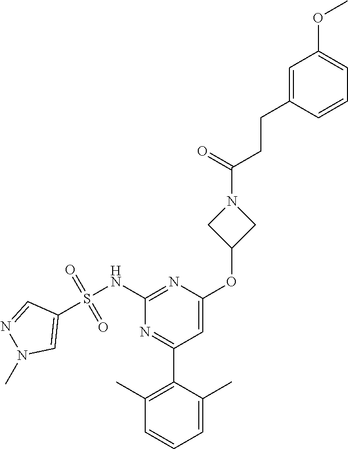 [SMILES: COc1cccc(CCC(=O)N2CC(Oc3cc(-c4c(C)cccc4C)nc(NS(=O)(=O)c4cnn(C)c4)n3)C2)c1] | 1.14 | 576.215 | 577.39 | R |
| 673 | 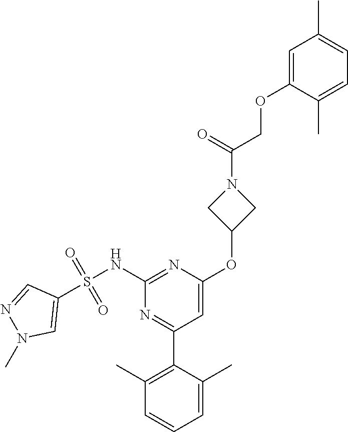 [SMILES: Cc1ccc(C)c(OCC(=O)N2CC(Oc3cc(-c4c(C)cccc4C)nc(NS(=O)(=O)c4cnn(C)c4)n3)C2)c1] | 1.75 | 576.215 | 577.32 | A |
| 674 | 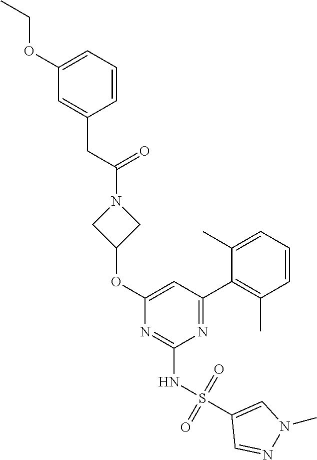 [SMILES: CCOc1cccc(CC(=O)N2CC(Oc3cc(-c4c(C)cccc4C)nc(NS(=O)(=O)c4cnn(C)c4)n3)C2)c1] | 1.6 | 576.215 | 577 | A |
| 675 | 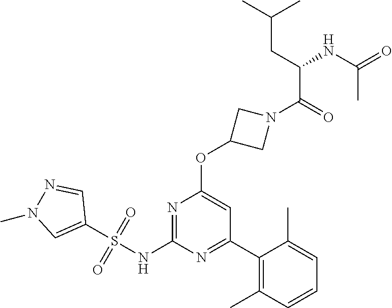 [SMILES: CC(=O)N[C@@H](CC(C)C)C(=O)N1CC(Oc2cc(-c3c(C)cccc3C)nc(NS(=O)(=O)c3cnn(C)c3)n2)C1] | 1.04 | 569.242 | 570.42 | R |
| 676 | 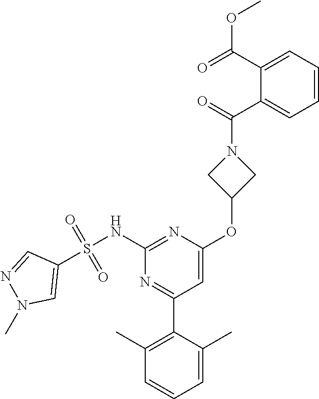 [SMILES: COC(=O)c1ccccc1C(=O)N1CC(Oc2cc(-c3c(C)cccc3C)nc(NS(=O)(=O)c3cnn(C)c3)n2)C1] | 1.06 | 576.179 | 577.35 | R |
| 677 | 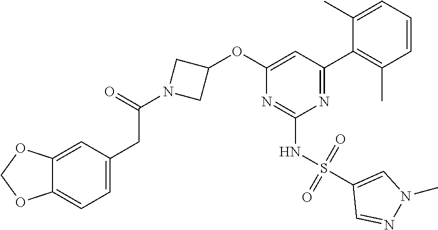 [SMILES: Cc1cccc(C)c1-c1cc(OC2CN(C(=O)Cc3ccc4c(c3)OCO4)C2)nc(NS(=O)(=O)c2cnn(C)c2)n1] | 1.08 | 576.179 | 577.32 | R |
| 678 | 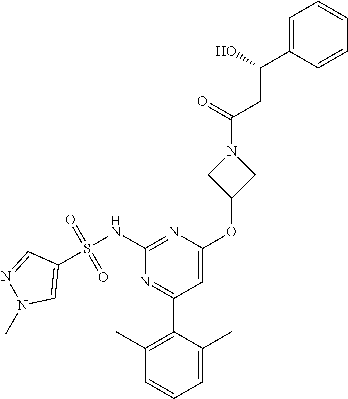 [SMILES: Cc1cccc(C)c1-c1cc(OC2CN(C(=O)C[C@H](O)c3ccccc3)C2)nc(NS(=O)(=O)c2cnn(C)c2)n1] | 1.02 | 562.2 | 563.35 | R |
| 679 | 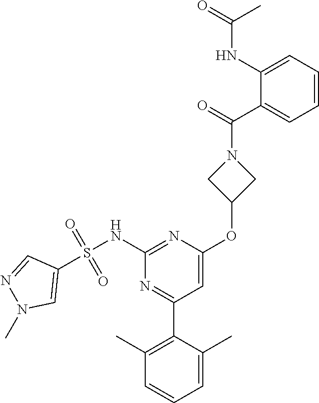 [SMILES: CC(=O)Nc1ccccc1C(=O)N1CC(Oc2cc(-c3c(C)cccc3C)nc(NS(=O)(=O)c3cnn(C)c3)n2)C1] | 1.03 | 575.195 | 576.34 | R |
| 680 | 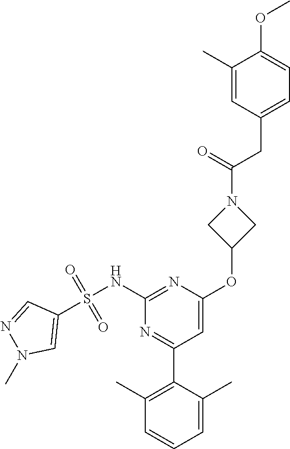 [SMILES: COc1ccc(CC(=O)N2CC(Oc3cc(-c4c(C)cccc4C)nc(NS(=O)(=O)c4cnn(C)c4)n3)C2)cc1C] | 1.18 | 576.215 | 577.39 | R |
| 681 | 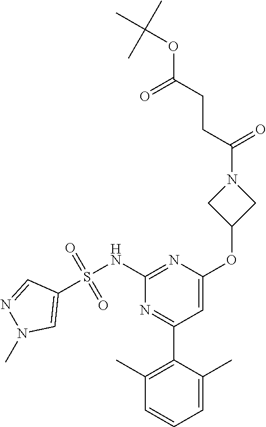 [SMILES: Cc1cccc(C)c1-c1cc(OC2CN(C(=O)CCC(=O)OC(C)(C)C)C2)nc(NS(=O)(=O)c2cnn(C)c2)n1] | 1.14 | 570.226 | 571.37 | R |
| 682 | 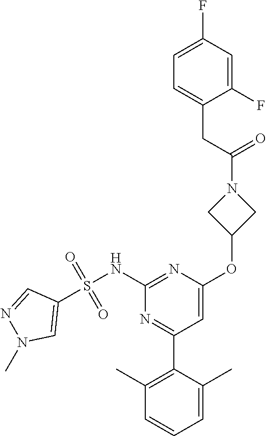 [SMILES: Cc1cccc(C)c1-c1cc(OC2CN(C(=O)Cc3ccc(F)cc3F)C2)nc(NS(=O)(=O)c2cnn(C)c2)n1] | 1.16 | 568.17 | 569.3 | R |
| 683 | 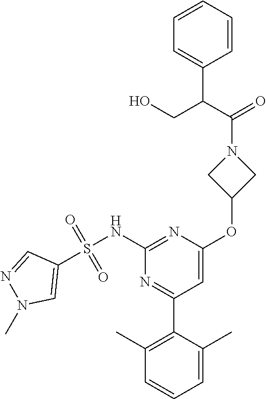 [SMILES: Cc1cccc(C)c1-c1cc(OC2CN(C(=O)C(CO)c3ccccc3)C2)nc(NS(=O)(=O)c2cnn(C)c2)n1] | 1.03 | 562.2 | 563.35 | R |
| 684 | 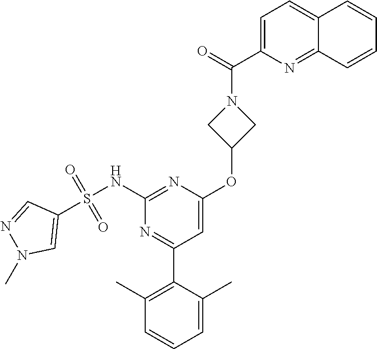 [SMILES: Cc1cccc(C)c1-c1cc(OC2CN(C(=O)c3ccc4ccccc4n3)C2)nc(NS(=O)(=O)c2cnn(C)c2)n1] | 1.24 | 569.185 | 570.35 | R |
| 685 | 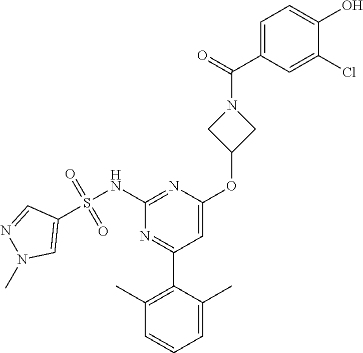 [SMILES: Cc1cccc(C)c1-c1cc(OC2CN(C(=O)c3ccc(O)c(Cl)c3)C2)nc(NS(=O)(=O)c2cnn(C)c2)n1] | 1.02 | 568.13 | 569.27 | R |
| 686 | 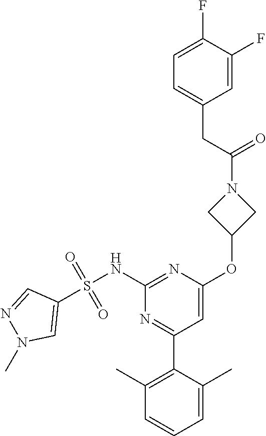 [SMILES: Cc1cccc(C)c1-c1cc(OC2CN(C(=O)Cc3ccc(F)c(F)c3)C2)nc(NS(=O)(=O)c2cnn(C)c2)n1] | 1.16 | 568.17 | 569.3 | R |
| 687 | 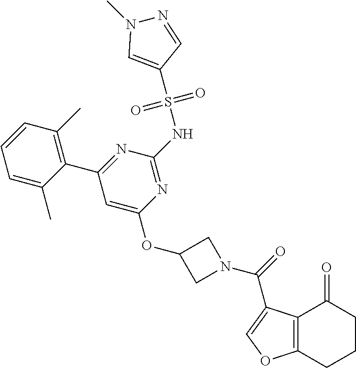 [SMILES: Cc1cccc(C)c1-c1cc(OC2CN(C(=O)c3coc4c3C(=O)CCC4)C2)nc(NS(=O)(=O)c2cnn(C)c2)n1] | 0.99 | 576.179 | 577.35 | R |
| 688 | 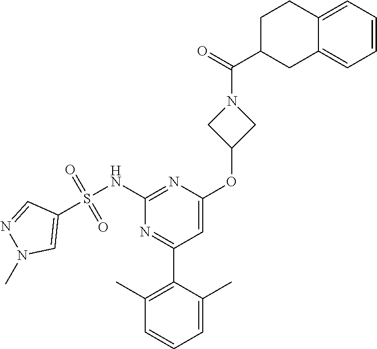 [SMILES: Cc1cccc(C)c1-c1cc(OC2CN(C(=O)C3CCc4ccccc4C3)C2)nc(NS(=O)(=O)c2cnn(C)c2)n1] | 1.26 | 572.221 | 573.36 | R |
| 689 | 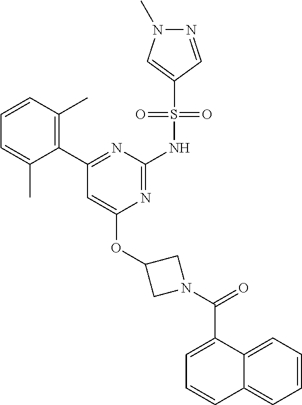 [SMILES: Cc1cccc(C)c1-c1cc(OC2CN(C(=O)c3cccc4ccccc34)C2)nc(NS(=O)(=O)c2cnn(C)c2)n1] | 1.12 | 569.185 | 570.35 | R |
| 690 | 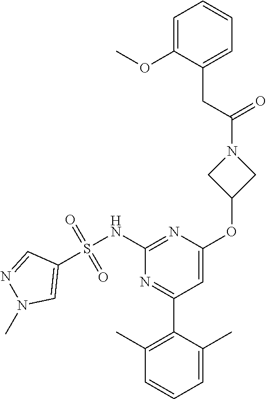 [SMILES: COc1ccccc1CC(=O)N1CC(Oc2cc(-c3c(C)cccc3C)nc(NS(=O)(=O)c3cnn(C)c3)n2)C1] | 1.13 | 562.2 | 563.35 | R |
| 691 | 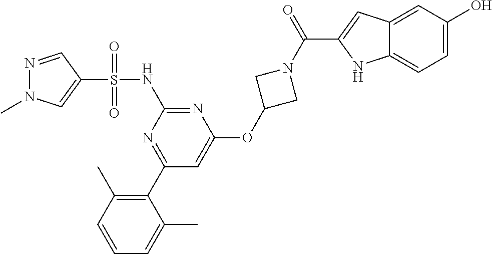 [SMILES: Cc1cccc(C)c1-c1cc(OC2CN(C(=O)c3cc4cc(O)ccc4[nH]3)C2)nc(NS(=O)(=O)c2cnn(C)c2)n1] | 0.97 | 573.179 | 574.34 | R |
| 692 | 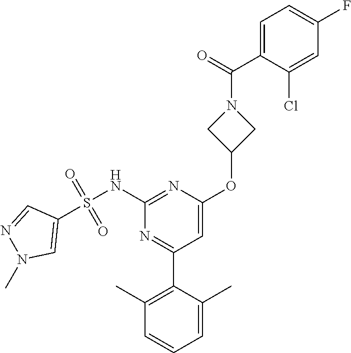 [SMILES: Cc1cccc(C)c1-c1cc(OC2CN(C(=O)c3ccc(F)cc3Cl)C2)nc(NS(=O)(=O)c2cnn(C)c2)n1] | 1.17 | 570.125 | 571.3 | R |
| 693 | 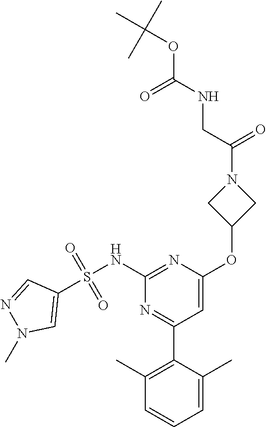 [SMILES: Cc1cccc(C)c1-c1cc(OC2CN(C(=O)CNC(=O)OC(C)(C)C)C2)nc(NS(=O)(=O)c2cnn(C)c2)n1] | 1.06 | 571.221 | 572.35 | R |
| 694 | 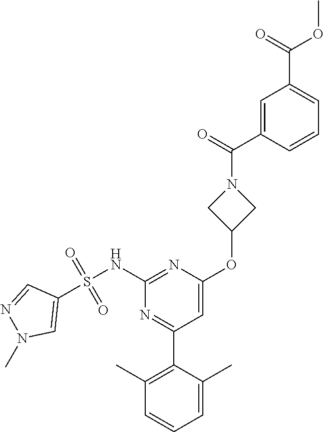 [SMILES: COC(=O)c1cccc(C(=O)N2CC(Oc3cc(-c4c(C)cccc4C)nc(NS(=O)(=O)c4cnn(C)c4)n3)C2)c1] | 1.12 | 576.179 | 577.35 | R |
| 695 | 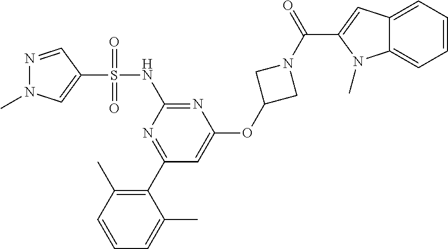 [SMILES: Cc1cccc(C)c1-c1cc(OC2CN(C(=O)c3cc4ccccc4n3C)C2)nc(NS(=O)(=O)c2cnn(C)c2)n1] | 1.27 | 571.2 | 572.35 | R |
| 696 | 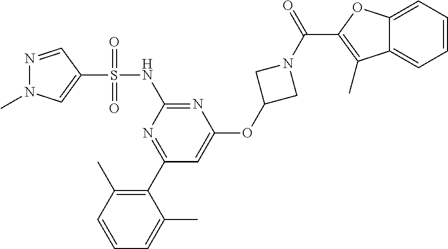 [SMILES: Cc1cccc(C)c1-c1cc(OC2CN(C(=O)c3oc4ccccc4c3C)C2)nc(NS(=O)(=O)c2cnn(C)c2)n1] | 1.32 | 572.184 | 573.33 | R |
| 697 | 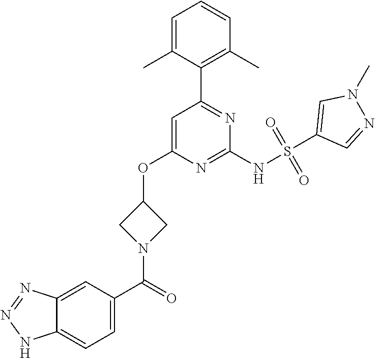 [SMILES: Cc1cccc(C)c1-c1cc(OC2CN(C(=O)c3ccc4[nH]nnc4c3)C2)nc(NS(=O)(=O)c2cnn(C)c2)n1] | 0.91 | 559.175 | 560.38 | R |
| 698 | 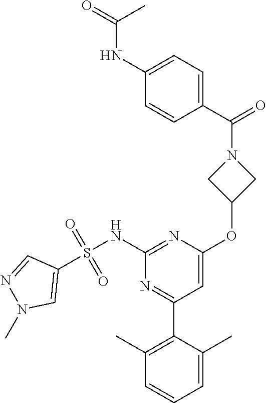 [SMILES: CC(=O)Nc1ccc(C(=O)N2CC(Oc3cc(-c4c(C)cccc4C)nc(NS(=O)(=O)c4cnn(C)c4)n3)C2)cc1] | 0.94 | 575.195 | 576.37 | R |
| 699 | 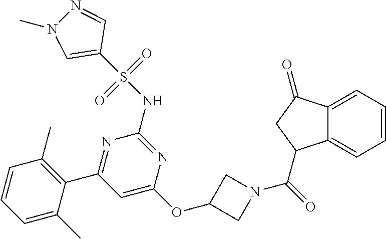 [SMILES: Cc1cccc(C)c1-c1cc(OC2CN(C(=O)C3CC(=O)c4ccccc43)C2)nc(NS(=O)(=O)c2cnn(C)c2)n1] | 1.03 | 572.184 | 573.33 | R |

| Compound | |
|---|---|
| number | NMR |
| 601 | ¹H NMR (400 MHz, DMSO-d₆) δ 8.24 (s, 1H), 7.71 (s, 1H), 7.29 − 7.21 (m, 3H), |
| | 7.12 (d, J = 7.6 Hz, 2H), 6.90 (d, J = 8.8 Hz, 2H), 6.44 (s, 1H), 5.14 (s, 1H), 4.26 |
| | (s, 1H), 3.93 (s, 1H), 3.84 (s, 3H), 3.74 (s, 4H), 3.41 (s, 1H), 2.08 (s, 3H), 1.98 (s, |
| | 6H), 1.32 (s, 2H), 0.98 (d, J = 38.9 Hz, 2H). |
| 603 | ¹H NMR (400 MHz, DMSO-d₆) δ 8.15 (s, 1H), 7.63 (s, 1H), 7.29 (t, J = 7.6 Hz, |
| | 1H), 7.26 − 7.21 (m, 1H), 7.15 − 7.07 (m, 6H), 6.42 (s, 1H), 5.04 (s, 1H), 4.26 (s, |
| | 1H), 4.08 (s, 2H), 3.83 (s, 6H), 3.56 (s, 4H), 3.33 (s, 1H), 2.32 (s, 4H), 2.23 (s, |
| | 3H), 2.08 (s, 1H), 2.00 (d, J = 17.2 Hz, 8H), 1.87 (s, 3H). |
| 604 | ¹H NMR (400 MHz, DMSO-d₆) δ 8.20 (s, 1H), 7.67 (s, 1H), 7.22 (dd, J = 19.1, |
| | 8.1 Hz, 3H), 7.12 (d, J = 7.6 Hz, 2H), 6.93 (d, J = 8.8 Hz, 2H), 6.43 (s, 1H), 5.09 |
| | (s, 1H), 4.23 (s, 1H), 3.84 (s, 4H), 3.73 (s, 5H), 3.37 (s, 1H), 2.08 (s, 2H), 1.97 (s, |
| | 6H), 1.41 (d, J = 8.6 Hz, 6H). |
| 607 | ¹H NMR (400 MHz, DMSO-d₆) δ 8.20 (s, 1H), 7.67 (s, 1H), 7.50 (td, J = 8.1, 1.8 |
| | Hz, 1H), 7.34 (tdd, J = 7.3, 5.0, 1.7 Hz, 1H), 7.26 − 7.16 (m, 3H), 7.12 (d, J = 7.6 |
| | Hz, 2H), 6.43 (s, 1H), 5.09 (s, 1H), 4.21 (s, 1H), 3.82 (d, J = 25.2 Hz, 5H), 3.24 |
| | (s, 1H), 2.08 (s, 6H), 1.97 (s, 6H), 1.89 − 1.66 (m, 4H), 1.44 (d, J = 62.0 Hz, 4H). |
| 609 | ¹H NMR (400 MHz, DMSO-d₆) δ 8.20 (s, 1H), 7.68 (s, 1H), 7.42 (d, J = 8.5 Hz, |
| | 2H), 7.29 (d, J = 8.5 Hz, 2H), 7.23 (t, J = 7.9 Hz, 1H), 7.12 (d, J = 7.6 Hz, 2H), |
| | 6.44 (s, 1H), 5.13 (s, 1H), 4.24 (s, 1H), 4.05 (s, 1H), 3.84 (s, 4H), 3.55 (d, J = 7.9 |
| | Hz, 1H), 2.32 (d, J = 5.9 Hz, 2H), 2.08 (s, 3H), 1.93 (d, J = 38.0 Hz, 8H), 1.64 (s, |
| | 4H). |
| 610 | ¹H NMR (400 MHz, DMSO-d₆) δ 8.19 (s, 1H), 7.67 (s, 1H), 7.36 (t, J = 7.6 Hz, |
| | 2H), 7.25 (dd, J = 18.6, 7.8 Hz, 4H), 7.12 (d, J = 7.6 Hz, 2H), 6.42 (s, 1H), 5.10 |
| | (s, 1H), 4.23 (s, 1H), 4.03 (s, 1H), 3.83 (s, 3H), 3.79 (d, J = 11.0 Hz, 1H), 3.48 − |
| | 3.45 (m, 1H), 2.33 (d, J = 8.4 Hz, 2H), 2.08 (s, 3H), 1.97 (s, 7H), 1.85 (s, 1H), |
| | 1.64 (s, 4H). |
| 617 | ¹H NMR (400 MHz, DMSO-d₆) δ 9.37 (d, J = 45.0 Hz, 1H), 8.30 (d, J = 7.0 Hz, |
| | 1H), 8.10 (dd, J = 19.9, 7.1 Hz, 2H), 7.78 (d, J = 14.1 Hz, 1H), 7.25 (s, 1H), 7.14 |
| | (d, J = 4.9 Hz, 2H), 6.91 (t, J = 6.8 Hz, 1H), 6.54 (s, 1H), 5.43 (d, J = 52.0 Hz, |
| | 1H), 4.78 − 4.40 (m, 3H), 3.85 (s, 3H), 3.55 (s, 3H), 2.01 (d, J = 8.1 Hz, 6H), 1.23 |
| | (d, J = 14.3 Hz, 3H). |
| 619 | ¹H NMR (400 MHz, DMSO-d₆) δ 8.28 (s, 1H), 8.15 (d, J = 7.6 Hz, 1H), 8.01 (d, |
| | J = 4.7 Hz, 1H), 7.78 (s, 1H), 7.29 − 7.21 (m, 1H), 7.14 (d, J = 7.6 Hz, 2H), 7.00 − |
| | 6.92 (m, 1H), 6.53 (s, 1H), 5.37 (s, 1H), 4.50 (d, J = 11.3 Hz, 3H), 4.16 (d, J = |
| | 10.5 Hz, 2H), 4.10 − 4.05 (m, 2H), 3.83 (s, 3H), 3.13 (s, 6H), 2.01 (s, 6H). |
| 628 | ¹H NMR (400 MHz, DMSO-d₆) δ 8.30 (s, 1H), 8.08 (d, J = 6.4 Hz, 1H), 7.80 (s, |
| | 1H), 7.30 (s, 1H), 7.27 − 7.23 (m, 1H), 7.14 (d, J = 7.6 Hz, 2H), 6.99 (d, J = 8.7 |
| | Hz, 1H), 6.54 (d, J = 8.5 Hz, 1H), 5.37 (s, 1H), 4.70 (s, 1H), 4.55 (s, 1H), 4.41 (s, |
| | 1H), 4.10 (d, J = 15.2 Hz, 2H), 3.91 (s, 1H), 3.85 (s, 3H), 3.28 (s, 7H), 2.01 (s, |
| | 7H). |
| 643 | ¹H NMR (400 MHz, DMSO-d₆) δ 8.30 (s, 1H), 7.77 (s, 1H), 7.29 − 7.21 (m, 1H), |
| | 7.14 (d, J = 7.7 Hz, 2H), 6.51 (s, 1H), 5.28 (s, 1H),4.52 (s, 1H), 4.26 (s, 1H), 4.12 |
| | (d, J = 13.7 Hz, 1H), 3.86 (s, 4H), 3.17 (s, 3H), 2.05 (d, J = 27.9 Hz, 9H), 1.50 (d, |
| | J = 16.0 Hz, 3H), 1.15 (d, J = 15.8 Hz, 2H), 0.86 (d, J = 6.6 Hz, 7H). |
| 647 | ¹H NMR (400 MHz, DMSO-d₆) δ 8.88 (d, J = 9.5 Hz, 1H), 8.80 (t, J = 6.7 Hz, |
| | 1H), 8.48 (dd, J = 16.2, 8.1 Hz, 1H), 8.29 (d, J = 20.1 Hz, 1H), 7.95 (s, 1H), 7.78 |
| | (d, J = 15.2 Hz, 1H), 7.29 − 7.19 (m, 1H), 7.17 − 7.11 (m, 2H), 6.51 (d, J = 21.0 |
| | Hz, 1H), 5.31 (d, J = 25.8 Hz, 1H), 4.68 (d, J = 44.9 Hz, 1H), 4.31 (d, J = 33.6 |
| | Hz, 2H), 3.86 (d, J = 14.4 Hz, 7H), 2.01 (d, J = 10.3 Hz, 7H), 1.83 − 1.69 (m, 1H), |
| | 0.89 − 0.74 (m, 3H). |

### Example 250: Preparation of Compound 700

### Step 1: N-[4-[l-[2-(2-chlorophenyl)ethyl]azetidin-3-yl]oxy-6-(2,6-dimethylphenyl)pyrimidin-2-yl]-1-methyl-pyrazole-4-sulfonamide

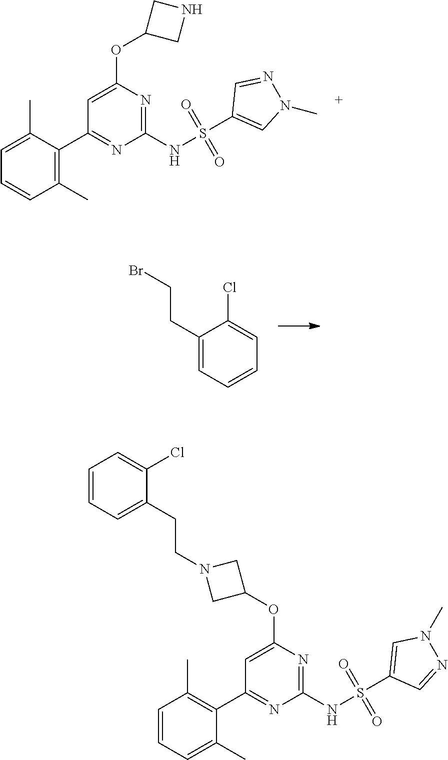 [SMILES: Cc1cccc(C)c1-c1cc(OC2CNC2)nc(NS(=O)(=O)c2cnn(C)c2)n1 ; Clc1ccccc1CCBr ; Cc1cccc(C)c1-c1cc(OC2CN(CCc3ccccc3Cl)C2)nc(NS(=O)(=O)c2cnn(C)c2)n1]

N-[4-(azetidin-3-yloxy)-6-(2,6-diinethylphenyl)pyrimidin-2-yl]-1-methyl-pyrazole-4 -sulfonamide (30 mg, 0.07238 mmol) in DMF (0.2 mL) was added to 1-(2-bromoethyl)-2-chloro-benzene (approximately 31.78 mg, 21.81 μL, 0.1448 mmol), followed by TEA (approximately 29.29 mg, 40.34 μL, 0.2895 mmol). The mixture was shaken at 60° C. for 1 hour. The reaction mixture was allowed to cool to room temperature, filtered and purified by reverse phase HPLC. N-[4-[1-[2-(2-chlorophenyl)ethyl]azetidin-3-yl]oxy-6-(2,6-dimethylphenyl)pyrimidin-2-yl]-1 -methyl-pyrazole-4-sulfonamide (3 mg). ESI-MS m/z calc. 552.171, found 554.0 (M+1)⁺; Retention time: 1.24 minutes; LC method A. ¹H NMR (400 MHz, DMSO-d₆) δ 11.38-11.00 (m, 1H), 8.55-8.28 (m, 1H), 7.87-7.75 (m, 1H), 7.55-7.45 (m, 2H), 7.41-7.32 (m, 2H), 7.30-7.22 (m, 1H), 7.15 (t, J=5.3 Hz, 2H), 6.68-6.49 (m, 1H), 5.45-5.15 (m, 1H), 4.91-4.61 (m, 2H), 4.60-4.42 (m, 1H), 4.39-4.24 (m, 2H), 3.92 (s, 3H), 3.89-3.84 (m, 1H), 3.09-2.93 (m, 2H), 2.02 (s, 6H)

### Example 251: Preparation of Compound 701

### Step 1: N-[4-(2,6-dimethylphenyl)-6-[1-[(3-methoxyphenyl)methyl]azetidin-3-yl]oxy-pyrimidin-2-yl]-1-methyl-pyrazole-4-sulfonamide

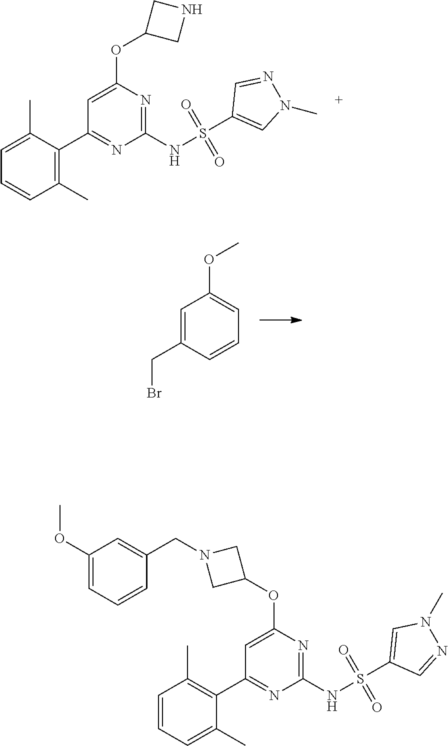

N-[4-(azetidin-3-yloxy)-6-(2,6-dimethylphenyl)pyrimidin-2-yl]-1-methyl-pyrazole-4-sulfonamide (30 mg, 0.07238 mmol) in DMF (0.2 mL) was added to 1-(bromomethyl)-3-methoxy-benzene (approximately 29.11 mg, 20.27 μL, 0.1448 mmol), followed by TEA (approximately 29.29 mg, 40.34 μL, 0.2895 mmol). The mixture was shaken at 60° C. for 1 hour. The reaction mixture was allowed to cool to room temperature, filtered and then purified by reverse phase HPLC. To give N-[4-(2,6-dimethylphenyl)-6-[1-[(3-methoxyphenyl)methyl]azetidin-3-yl]oxy-pyrimidin-2-yl]-1-methyl-pyrazole-4-sulfonamide (5.3 mg). ESI-MS m/z calc. 534.2049, found 535.32 (M+1)⁺; Retention time: 1.27 minutes; LC method A. ¹H NMR (400 MHz, DMSO-d₆) δ 11.47 (s, 1H), 8.50 (s, 1H), 7.80 (d, J=16.7 Hz, 1H), 7.40-7.34 (m, 1H), 7.28-7.21 (m, 2H), 7.14 (d, J=7.7 Hz, 4H), 7.01 (d, J=10.4 Hz, 1H), 6.55 (s, 1H), 5.20 (t, J=7.0 Hz, 1H), 4.55-4.41 (m, 4H), 4.28 (d, J=40.4 Hz, 2H), 3.90 (s, 3H), 3.80 (s, 3H), 2.01 (s, 6H).

### Example 252: Preparation of Compound 702

### Step 1: N-[4-(2,6-dimethylphenyl)-6-[1-[3-(m-tolyl)propyl]azetidin-3-yl]oxy-pyrimidin-2-yl]-1-methyl-pyrazole-4-sulfonamide

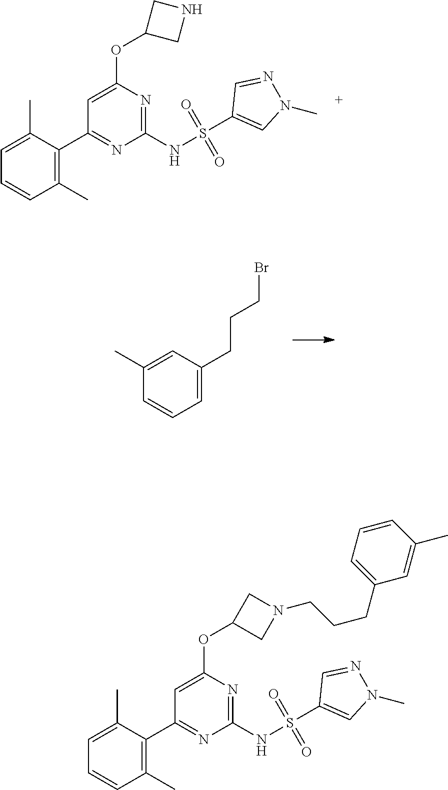

N-[4-(azetidin-3-yloxy)-6-(2,6-dimethylphenyl)pyrimidin-2-yl]-1-methyl-pyrazole-4-sulfonamide (30 mg, 0.07238 mmol) in DMF (0.2 mL) was added to 1-(3-bromopropyl)-3 -methyl-benzene (approximately 30.86 mg, 0.1448 mmol), followed by TEA (approximately 29.29 mg, 40.34 μL, 0.2895 mmol). The mixture was shaken at 60° C. for 1 hour. The reaction mixture was allowed to cool to room temperature, filtered and purified by reverse phase HPLC to give N-[4-(2,6-dimethylphenyl)-6-[1-[3-(m-tolyl)propyl]azetidin-3-yl]oxy-pyrimidin-2-yl]-1 -methyl-pyrazole-4-sulfonamide (2.3 mg). ESI-MS m/z calc. 546.24133, found 547.36 (M+1)⁺; Retention time: 1.54 minutes; LC method A. ¹H NMR (400 MHz, DMSO-d₆) δ 11.77 (s, 1H), 10.73 (d, J=101.4 Hz, 1H), 8.38 (d, J=59.5 Hz, 1H), 7.82 (d, J=13.3 Hz, 1H), 7.23 (dt, J=23.6, 7.4 Hz, 2H), 7.14 (d, J=7.6 Hz, 2H), 7.08-7.00 (m, 3H), 6.57 (s, 1H), 5.39-5.15 (m, 1H), 4.64 (s, 1H), 4.32 (d, J=81.8 Hz, 3H), 3.89 (d, J=19.5 Hz, 3H), 3.23 (s, 2H), 2.61 (s, 2H), 2.29 (s, 3H), 2.01 (s, 6H), 1.81 (s, 2H).

### Example 253: Preparation of Compound 703

### Step 1: N-[4-[1-[2-(4-chlorophenyl)ethyl]azetidin-3-yl]oxy-6-(2,6-dimethylphenyl)pyrimidin-2-yl]-1-methyl-pyrazole-4-sulfonamide

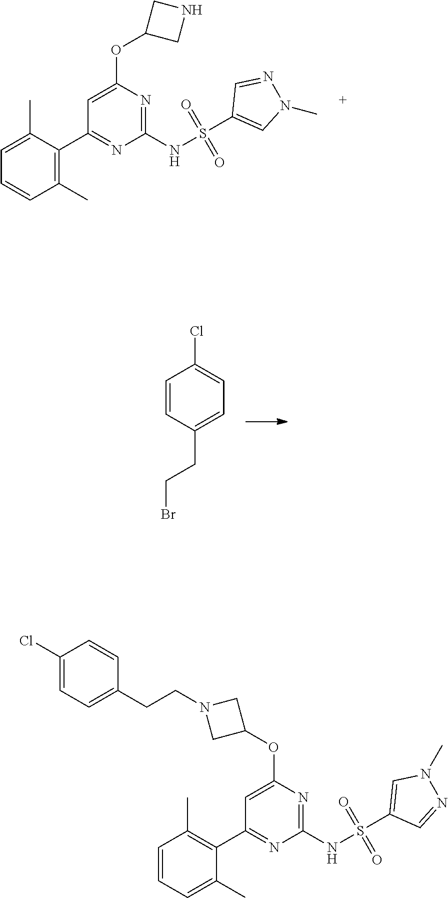 [SMILES: Cc1cccc(C)c1-c1cc(OC2CNC2)nc(NS(=O)(=O)c2cnn(C)c2)n1 ; Clc1ccc(CCBr)cc1 ; Cc1cccc(C)c1-c1cc(OC2CN(CCc3ccc(Cl)cc3)C2)nc(NS(=O)(=O)c2cnn(C)c2)n1]

N-[4-(azetidin-3-yloxy)-6-(2,6-dimethylphenyl)pyrimidin-2-yl]-1-methyl-pyrazole-4-sulfonamide (30 mg, 0.07238 mmol) in DMF (0.2 mL) was added to 1-(2-bromoethyl)-4-chloro-benzene (approximately 31.78 mg, 21.07 μL, 0.1448 mmol), followed by TEA (approximately 29.29 mg, 40.34 μL, 0.2895 mmol). The mixture was shaken at 60° C. for 1 hour. The reaction mixture was allowed to cool to room temperature, filtered and purified by reverse phase HPLC to give N-[4-[1-[2-(4-chlorophenyl)ethyl]azetidin-3-yl]oxy-6-(2,6-dimethylphenyl)pyrimidin-2-yl]-1-methyl-pyrazole-4-sulfonamide (3.5 mg). ESI-MS m/z calc. 552.171, found 554.0 (M+1)⁺; Retention time: 1.27 minutes; LC method A. ¹H NMR (400 MHz, DMSO-d₆) δ 11.80 (s, 1H), 8.60-8.28 (m, 1H), 7.89-7.73 (m, 1H), 7.46-7.40 (m, 2H), 7.38-7.33 (m, 2H), 7.29-7.22 (m, 1H), 7.19-7.11 (m, 2H), 6.70-6.51 (m, 1H), 5.34-5.15 (m, 1H), 4.63 (s, 2H), 4.47 (s, 1H), 4.39-4.22 (m, 2H), 3.92 (s, 3H), 3.87 (s, 1H), 2.94-2.84 (m, 2H), 2.09-1.96 (m, 6H).

### Example 254: Preparation of 704

### Step 1: N-[4-(2,6-Dimethylphenyl)-6-[1-[[3-(trifluoromethyl)phenyl]methyl]azetidin-3-yl]oxy-pyrimidin-2-yl]-1-methyl-pyrazole-4-sulfonamide

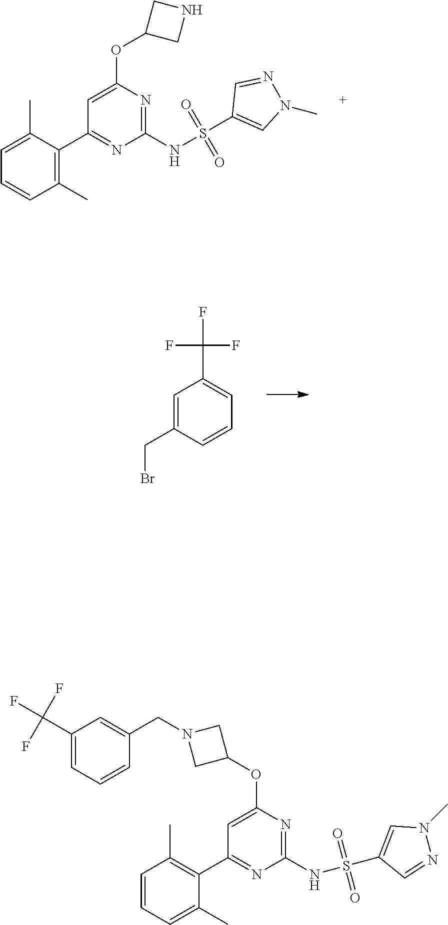 [SMILES: Cc1cccc(C)c1-c1cc(OC2CNC2)nc(NS(=O)(=O)c2cnn(C)c2)n1 ; FC(F)(F)c1cccc(CBr)c1 ; Cc1cccc(C)c1-c1cc(OC2CN(Cc3cccc(C(F)(F)F)c3)C2)nc(NS(=O)(=O)c2cnn(C)c2)n1]

N-[4-(azetidin-3-yloxy)-6-(2,6-dimethylphenyl)pyrimidin-2-yl]-1-methyl-pyrazole-4-sulfonamide (30 mg, 0.07238 mmol) in DMF (0.2 mL) was added to 1-(bromomethyl)-3-(trifluoromethyl)benzene (approximately 34.61 mg, 22.12 μL, 0.1448 mmol), followed by TEA (approximately 29.29 mg, 40.34 μL, 0.2895 mmol). The mixture was shaken at 60° C. for 1 hour. The reaction mixture was allowed to cool to room temperature, filtered and purified by reverse phase HPLC to give N-[4-(2,6-dimethylphenyl)-6-[1-[[3-(trifluoromethyl)phenyl]methyl]azetidin-3-yl]oxy-pyrimidin-2-yl]-1-methyl-pyrazole-4-sulfonamide (6.4 mg). ESI-MS m/z calc. 572.18176, found 573.31 (M+1)⁺; Retention time: 1.4 minutes; LC method A. ¹H NMR (400 MHz, DMSO-d₆) δ 11.63 (s, 1H), 8.48 (s, 1H), 8.03 (d, J=2.0 Hz, 1H), 7.95-7.91 (m, 1H), 7.85-7.83 (m, 1H), 7.77 (s, 1H), 7.74-7.70 (m, 1H), 7.28 -7.24 (m, 1H), 7.16-7.13 (m, 2H), 6.57 (s, 1H), 5.26-5.15 (m, 1H), 4.57-4.50 (m, 2H), 4.44 -4.37 (m, 2H), 3.89 (s, 3H), 3.87-3.82 (m, 2H), 2.01 (s, 6H).

### Example 255: Characterization of Compounds 705-724

The compounds in the following tables were prepared in a manner analogous to that described above using commercially available reagents and intermediates described herein.

| | | LCMS | | | |
|---|---|---|---|---|---|
| | | Room | | | |
| Compound | | temperatue | Calc. | | LCMS |
| Number | Structure | (min) | mass | M + 1 | Method |
| 705 | 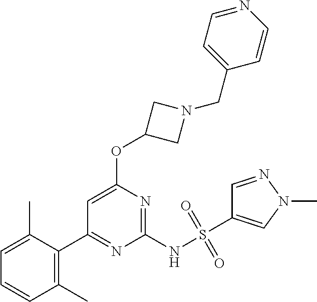 [SMILES: Cc1cccc(C)c1-c1cc(OC2CN(Cc3ccncc3)C2)nc(NS(=O)(=O)c2cnn(C)c2)n1] | 0.76 | 505.19 | 506 | A |
| 706 | 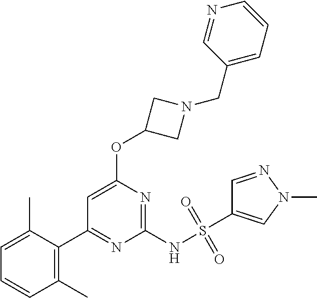 [SMILES: Cc1cccc(C)c1-c1cc(OC2CN(Cc3cccnc3)C2)nc(NS(=O)(=O)c2cnn(C)c2)n1] | 0.79 | 505.19 | 506 | A |
| 707 | 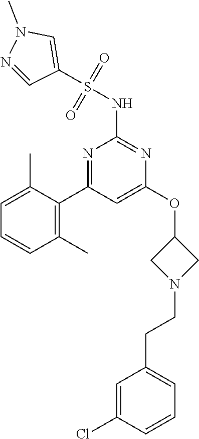 [SMILES: Cc1cccc(C)c1-c1cc(OC2CN(CCc3cccc(Cl)c3)C2)nc(NS(=O)(=O)c2cnn(C)c2)n1] | 1.27 | 552.171 | 554 | A |
| 708 | 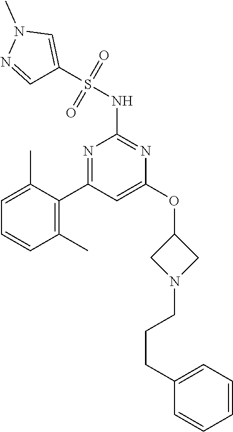 [SMILES: Cc1cccc(C)c1-c1cc(OC2CN(CCCc3ccccc3)C2)nc(NS(=O)(=O)c2cnn(C)c2)n1] | 1.31 | 532.226 | 533 | A |
| 709 | 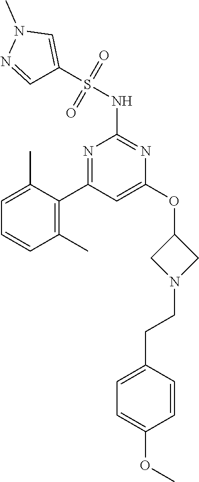 [SMILES: COc1ccc(CCN2CC(Oc3cc(-c4c(C)cccc4C)nc(NS(=O)(=O)c4cnn(C)c4)n3)C2)cc1] | 1.17 | 548.221 | 549 | A |
| 710 | 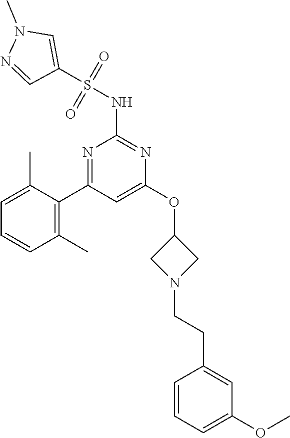 [SMILES: COc1cccc(CCN2CC(Oc3cc(-c4c(C)cccc4C)nc(NS(=O)(=O)c4cnn(C)c4)n3)C2)c1] | 1.17 | 548.221 | 549 | A |
| 711 | 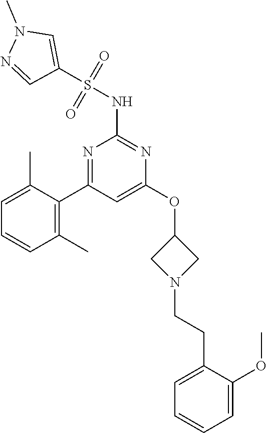 [SMILES: COc1ccccc1CCN1CC(Oc2cc(-c3c(C)cccc3C)nc(NS(=O)(=O)c3cnn(C)c3)n2)C1] | 1.36 | 548.221 | 549.36 | A |
| 712 | 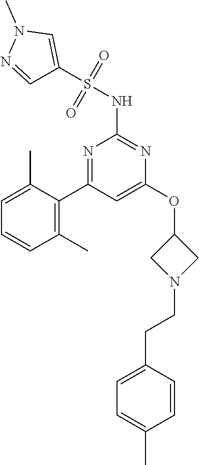 [SMILES: Cc1ccc(CCN2CC(Oc3cc(-c4c(C)cccc4C)nc(NS(=O)(=O)c4cnn(C)c4)n3)C2)cc1] | 1.39 | 532.226 | 533.32 | A |
| 713 | 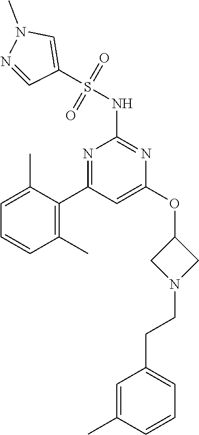 [SMILES: Cc1cccc(CCN2CC(Oc3cc(-c4c(C)cccc4C)nc(NS(=O)(=O)c4cnn(C)c4)n3)C2)c1] | 1.33 | 532.226 | 533 | A |
| 714 | 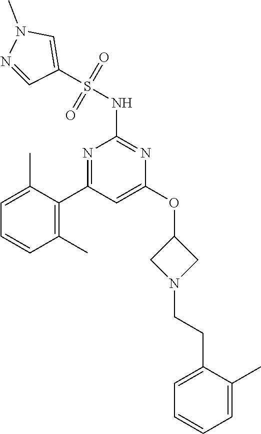 [SMILES: Cc1ccccc1CCN1CC(Oc2cc(-c3c(C)cccc3C)nc(NS(=O)(=O)c3cnn(C)c3)n2)C1] | 1.37 | 532.226 | 533.36 | A |
| 715 | 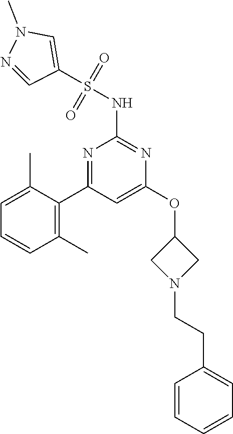 [SMILES: Cc1cccc(C)c1-c1cc(OC2CN(CCc3ccccc3)C2)nc(NS(=O)(=O)c2cnn(C)c2)n1] | 1.32 | 518.21 | 519.32 | A |
| 716 | 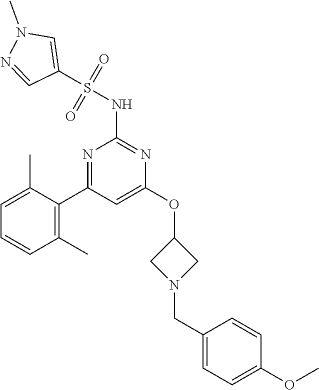 [SMILES: COc1ccc(CN2CC(Oc3cc(-c4c(C)cccc4C)nc(NS(=O)(=O)c4cnn(C)c4)n3)C2)cc1] | 1.28 | 534.205 | 535.32 | A |
| 717 | 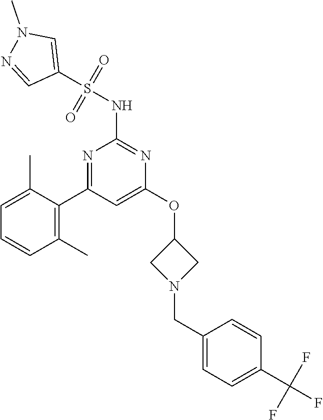 [SMILES: Cc1cccc(C)c1-c1cc(OC2CN(Cc3ccc(C(F)(F)F)cc3)C2)nc(NS(=O)(=O)c2cnn(C)c2)n1] | 1.42 | 572.182 | 573.31 | A |
| 718 | 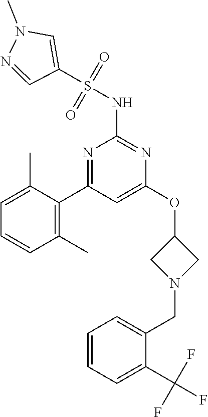 [SMILES: Cc1cccc(C)c1-c1cc(OC2CN(Cc3ccccc3C(F)(F)F)C2)nc(NS(=O)(=O)c2cnn(C)c2)n1] | 1.37 | 572.182 | 573.31 | A |
| 719 | 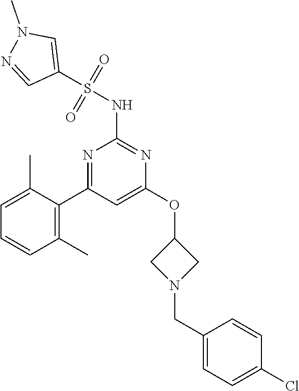 [SMILES: Cc1cccc(C)c1-c1cc(OC2CN(Cc3ccc(Cl)cc3)C2)nc(NS(=O)(=O)c2cnn(C)c2)n1] | 1.35 | 538.155 | 539.28 | A |
| 720 | 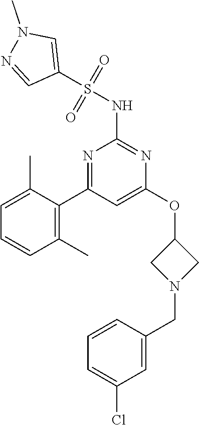 [SMILES: Cc1cccc(C)c1-c1cc(OC2CN(Cc3cccc(Cl)c3)C2)nc(NS(=O)(=O)c2cnn(C)c2)n1] | 1.33 | 538.155 | 539.28 | A |
| 721 | 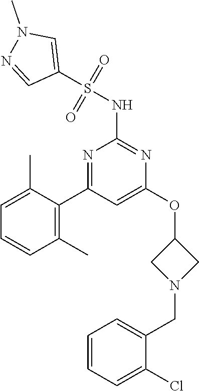 [SMILES: Cc1cccc(C)c1-c1cc(OC2CN(Cc3ccccc3Cl)C2)nc(NS(=O)(=O)c2cnn(C)c2)n1] | 1.3 | 538.155 | 539.28 | A |
| 722 | 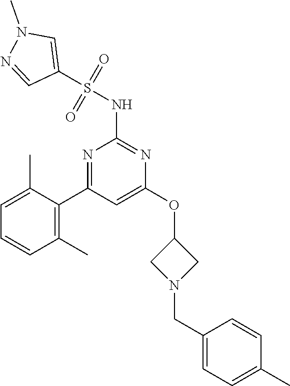 [SMILES: Cc1ccc(CN2CC(Oc3cc(-c4c(C)cccc4C)nc(NS(=O)(=O)c4cnn(C)c4)n3)C2)cc1] | 1.32 | 518.21 | 519.32 | A |
| 723 | 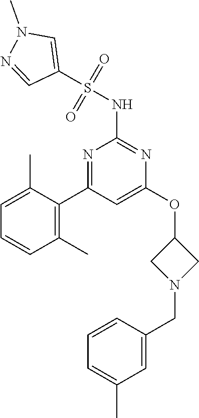 [SMILES: Cc1cccc(CN2CC(Oc3cc(-c4c(C)cccc4C)nc(NS(=O)(=O)c4cnn(C)c4)n3)C2)c1] | 1.33 | 518.21 | 519.32 | A |
| 724 | 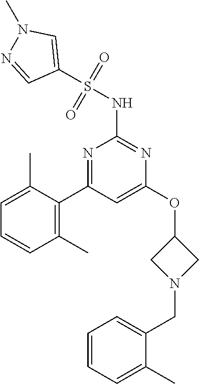 [SMILES: Cc1ccccc1CN1CC(Oc2cc(-c3c(C)cccc3C)nc(NS(=O)(=O)c3cnn(C)c3)n2)C1] | 1.28 | 518.21 | 519.32 | A |
| Compound | | | | | |
| Number | NMR | | | | |

| | |
|---|---|
| 707 | ¹H NMR (400 MHz, DMSO-d₆) δ 8.47 (s, 1H), 7.80 (s, 1H), 7.46 (s, 1H), 7.37 (d, |
| | J = 13.2 Hz, 3H), 7.28 (d, J = 17.0 Hz, 3H), 7.14 (d, J = 7.6 Hz, 2H), 6.58 (s, 1H), |
| | 5.21 (s, 1H), 4.66 (s, 2H), 4.47 (s, 1H), 4.34 (s, 3H), 3.90 (d, J = 20.8 Hz, 4H), |
| | 3.59 (s, 3H), 2.89 (s, 2H), 2.01 (s, 7H). |
| 708 | ¹H NMR (400 MHz, DMSO-d₆) δ 9.11 (s, 1H), 8.91 (s, 1H), 8.46 (s, 1H), 7.90 (s, |
| | 1H), 7.36-7.31 (m, 2H), 7.28-7.19 (m, 5H), 7.11 (d, J = 7.6 Hz, 2H), 5.96 (s, |
| | 1H), 4.98 (s, 1H), 4.30 (d, J = 11.8 Hz, 2H), 3.86 (d, J = 1.8 Hz, 3H), 3.62 (s, 1H), |
| | 3.49 (s, 1H), 3.05 (d, J = 14.8 Hz, 2H), 2.72-2.67 (m, 2H), 2.02 (s, 8H). |
| 718 | ¹H NMR (400 MHz, DMSO-d₆) δ 8.41 (d, J = 10.9 Hz, 1H), 7.91-7.76 (m, 4H), |
| | 7.67 (t, J = 7.6 Hz, 1H), 7.39-7.25 (m, 1H), 7.19 (dd, J = 19.9, 7.6 Hz, 3H), 6.03 |
| | (d, J = 16.7 Hz, 1H), 4.82 (d, J = 8.7 Hz, 1H), 4.50-4.32 (m, 4H), 4.30-4.22 (m, |
| | 1H), 3.86 (s, 3H), 3.73 (s, 1H), 2.25 (s, 6H), 2.04 (s, 2H). |
| 719 | ¹H NMR (400 MHz, DMSO-d₆) δ 8.51 (s, 1H), 7.77 (s, 1H), 7.63 (d, J = 13.4 Hz, |
| | 2H), 7.55 (t, J = 5.6 Hz, 2H), 7.26 (d, J = 14.9 Hz, 1H), 7.14 (d, J = 7.5 Hz, 2H), |
| | 6.53 (d, J = 23.6 Hz, 1H), 5.46-5.12 (m, 1H), 4.51 (s, 3H), 4.27 (d, J = 51.8 Hz, |
| | 2H), 3.88 (d, J = 16.1 Hz, 3H), 2.01 (s, 6H). |
| 720 | ¹H NMR (400 MHz, DMSO-d₆) δ 8.37 (d, J = 57.7 Hz, 1H), 7.80 (d, J = 17.3 Hz, |
| | 1H), 7.68 (d, J = 26.2 Hz, 1H), 7.56 (d, J = 13.5 Hz, 3H), 7.26 (t, J = 7.5 Hz, 1H), |
| | 7.15 (d, J = 7.6 Hz, 2H), 6.54 (d, J = 23.3 Hz, 1H), 5.19 (s, 1H), 4.51 (d, J = 26.7 |
| | Hz, 3H), 4.32 (d, J = 56.5 Hz, 2H), 3.87 (d, J = 12.6 Hz, 3H), 2.01 (s, 6H). |

### Example 256: Preparation of Compound 725

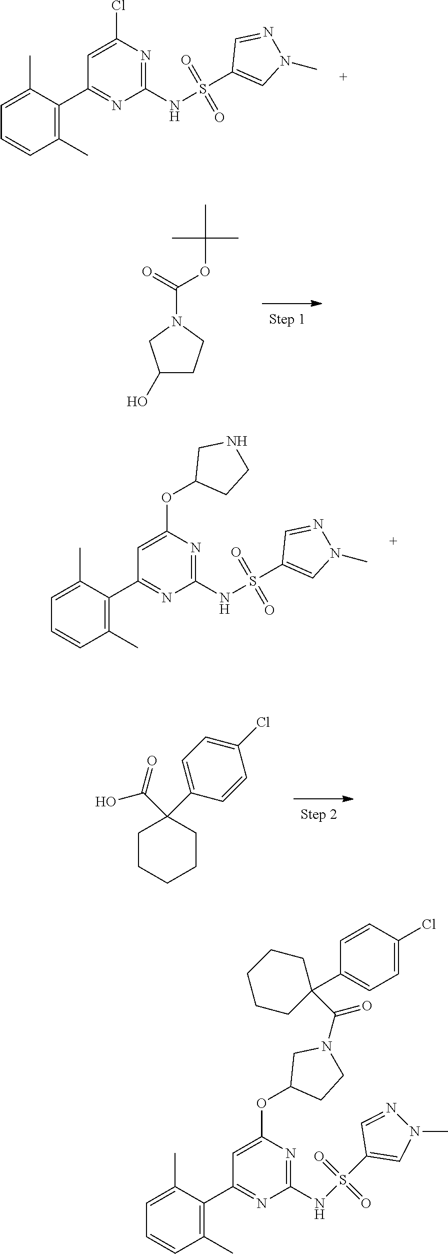 [SMILES: Cc1cccc(C)c1-c1cc(Cl)nc(NS(=O)(=O)c2cnn(C)c2)n1 ; CC(C)(C)OC(=O)N1CCC(O)C1 ; Cc1cccc(C)c1-c1cc(OC2CCNC2)nc(NS(=O)(=O)c2cnn(C)c2)n1 ; O=C(O)C1(c2ccc(Cl)cc2)CCCCC1 ; Cc1cccc(C)c1-c1cc(OC2CCN(C(=O)C3(c4ccc(Cl)cc4)CCCCC3)C2)nc(NS(=O)(=O)c2cnn(C)c2)n1]

### Step 1: N-[4-(2,6-Dimethylphenyl)-6-pyrrolidin-3-yloxy-pyrimidin-2-yl]-1-methyl-pyrazole-4-sulfonamide

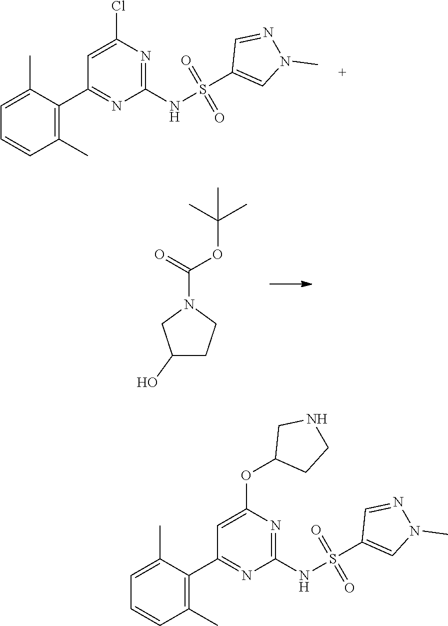 [SMILES: Cc1cccc(C)c1-c1cc(Cl)nc(NS(=O)(=O)c2cnn(C)c2)n1 ; CC(C)(C)OC(=O)N1CCC(O)C1 ; Cc1cccc(C)c1-c1cc(OC2CCNC2)nc(NS(=O)(=O)c2cnn(C)c2)n1]

NaH (698 mg of 60% w/w, 17.45 mmol) was added to tert-butyl 3-hydroxypyrrolidine-1-carboxylate (2.94 g, 15.70 mmol) in NMP (15 mL) at 0° C. The mixture was stirred for 1 hour. then added to N-[4-chloro-6-(2,6-dimethylphenyl)pyrimidin-2-yl]-1 -methyl-pyrazole-4-sulfonamide (3 g, 7.940 mmol) in NMP (5 mL). The resulting mixture was stirred at 105° C. for 10 minutes. The reaction mixture was cooled down to room temperature. The pH of the mixture was adjusted to ˜5 with 1 N HCl, extracted with ethyl acetate (3×20 mL). The combined organic layers were washed with brine, dried over Na₂SO₄, filtered, concentrated and purified on silica using a gradient of ethyl acetate and hexane. The product came out ˜50% ethyl acetate. tert-Butyl 3-[6-(2,6-dimethylphenyl)-2-[(1-methylpyrazol-4-yl)sulfonylamino]pyrimidin-4-yl]oxypyrrolidine-1-carboxylate (4.147 g, 98%). ESI-MS m z calc. 528.2155, found 529.0 (M+1)⁺; Retention time: 1.68 minutes (LC method A). tert-Butyl 3-[6-(2,6-dimethylphenyl)-2-[(1-methylpyrazol-4-yl)sulfonylamino]pyrimidin-4-yl]oxypyrrolidine-1-carboxylate (4.147 g, 98%) was stirred in TFA (10 mL, 129.8 mmol)/DCM (20 mL) at room temperature for 30 minutes. The reaction mixture was concentrated to remove solvents. The crude was redissolved in DCM/toluene, concentrated (repeated 3 times) and used as is in the next step without further purification. N-[4-(2,6-dimethylphenyl)-6-pyrrolidin-3-yloxy-pyrimidin-2-yl]-1-methyl-pyrazole-4-sulfonamide (3.35 g, 95%). ¹H NMR (400 MHz, DMSO-d₆) δ 9.49 (s, 1H), 9.31 (s, 1H), 8.33 (s, 1H), 7.79 (s, 1H), 7.30-7.20 (m, 1H), 7.13 (d, J=7.6 Hz, 2H), 6.39 (s, 1H), 5.53-5.40 (m, 1H), 3.87 (s, 3H), 3.41-3.23 (m, 4H), 2.40-2.27 (m, 1H), 2.20-2.09 (m, 1H), 2.02 (s, 6H). ESI-MS m/z calc. 428.16306, found 429.0 (M+1)⁺; Retention time: 0.74 minutes (LC method A).

### Step 2: N-[4-[1-[1-(4-chlorophenyl)cyclohexanecarbonyl]pyrrolidin-3-yl]oxy-6-(2,6-dimethylphenyl)pyrimidin-2-yl]-1-methyl-pyrazole-4-sulfonamide

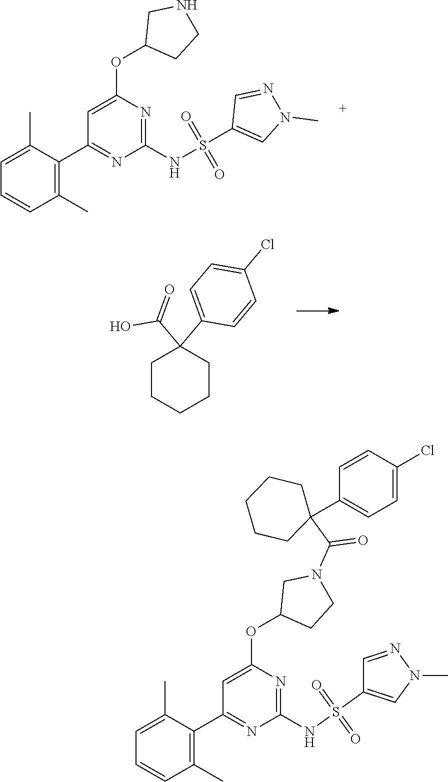 [SMILES: Cc1cccc(C)c1-c1cc(OC2CCNC2)nc(NS(=O)(=O)c2cnn(C)c2)n1 ; O=C(O)C1(c2ccc(Cl)cc2)CCCCC1 ; Cc1cccc(C)c1-c1cc(OC2CCN(C(=O)C3(c4ccc(Cl)cc4)CCCCC3)C2)nc(NS(=O)(=O)c2cnn(C)c2)n1]

N-[4-(2,6-dimethylphenyl)-6-pyrrolidin-3-yloxy-pyrimidin-2-yl]-1-methyl-pyrazole-4-sulfonamide (30 mg, 0.07001 mmol) in DMF (0.2 mL) was added to 1-(4-chlorophenyl)cyclohexanecarboxylic acid (approximately 25.06 mg, 0.1050 mmol) followed by HATU (approximately 39.92 mg, 0.1050 mmol), and K₂CO₃ (approximately 38.70 mg, 0.2800 mmol). The reaction mixture was shaken for 16 hours at 70° C. The reaction mixture was filtered and purified by reverse phase HPLC to give N-[4-[1-[1-(4-chlorophenyl)cyclohexanecarbonyl]pyrrolidin-3-yl]oxy-6-(2,6-dimethylphenyl)pyrimidin-2-yl]-1-methyl-pyrazole-4-sulfonamide (36.5 mg, 80%). ESI-MS m/z calc. 648.2286, found 649.36 (M+1)⁺; Retention time: 1.93 minutes; LC method A. ¹H NMR (400 MHz, DMSO-d₆) δ 11.61 (s, 1H), 8.26 (s, 1H), 7.73 (s, 1H), 7.40 (s, 1H), 7.36-7.18 (m, 4H), 7.13 (d, J=7.5 Hz, 1H), 6.54-6.03 (m, 1H), 5.43-5.11 (m, 1H), 3.85 (s, 3H), 3.77-3.59 (m, 2H), 3.18-2.94 (m, 2H), 2.39-2.17 (m, 3H), 2.13-1.98 (m, 6H), 1.94-1.22 (m, 10H).

### Example 257: Preparation of Compound 726

### Step 1: N-[4-(2,6-Dimethylphenyl)-6-[1-(2-pyrrol-1-ylbenzoyl)pyrrolidin-3-yl]oxy-pyrimidin-2-yl]-1-methyl-pyrazole-4-sulfonamide

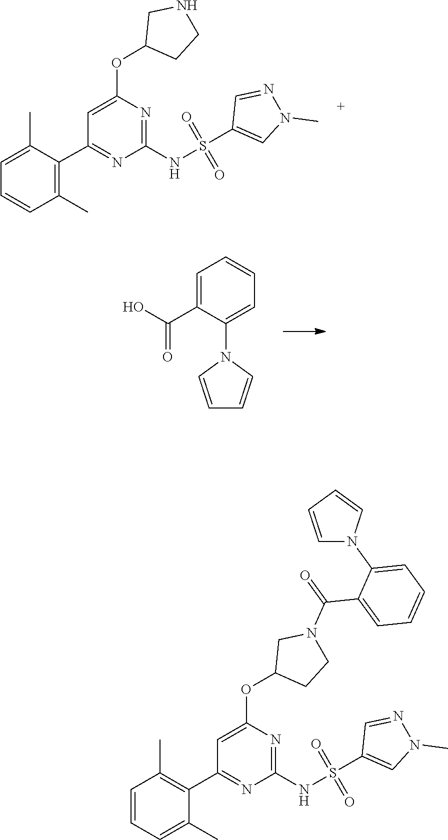 [SMILES: Cc1cccc(C)c1-c1cc(OC2CCNC2)nc(NS(=O)(=O)c2cnn(C)c2)n1 ; O=C(O)c1ccccc1-n1cccc1 ; Cc1cccc(C)c1-c1cc(OC2CCN(C(=O)c3ccccc3-n3cccc3)C2)nc(NS(=O)(=O)c2cnn(C)c2)n1]

N-[4-(2,6-dimethylphenyl)-6-pyrrolidin-3-yloxy-pyrimidin-2-yl]-1-methyl-pyrazole-4-sulfonamide (30 mg, ) in DMF (0.2 mL) was added to 2-pyrrol-1-ylbenzoic acid, followed by HATU (in DMF (0.2 mL) ), and K₂CO₃. The reaction mixture was shaken for 16 hours at 70° C. The reaction mixture was filtered and purified by reverse phase HPLC to give N-[4-(2,6-dimethylphenyl)-6-[1-(2-pyrrol-1-ylbenzoyl)pyrrolidin-3-yl]oxy-pyrimidin-2-yl]-1-methyl-pyrazole-4-sulfonamide (11.8 mg). ESI-MS m/z calc. 597.2158, found 598.39 (M+1)⁺; Retention time: 1.16 minutes; LC method A. ¹H NMR (400 MHz, DMSO-d₆) δ 8.37-8.11 (m, 1H), 7.79-7.64 (m, 1H), 7.61-7.53 (m, 1H), 7.53-7.48 (m, 1H), 7.48-7.44 (m, 1H), 7.44-7.32 (m, 1H), 7.30-7.20 (m, 1H), 7.20-7.09 (m, 2H), 7.04-6.96 (m, 1H), 6.93 (s, 1H), 6.50-5.83 (m, 3H), 5.45-5.20 (m, 1H), 3.87 (s, 3H), 3.77 (s, 2H), 3.75-3.64 (m, 1H), 3.56-3.35 (m, 2H), 2.09-1.95 (m, 7H), 1.94-1.79 (m, 1H).

### Example 258: Preparation of Compound 727

### Step 1: N-[4-[1-[1-(4-chlorophenyl)cyclopentanecarbonyl]pyrrolidin-3-yl]oxy-6-(2,6-dimethylphenyl)pyrimidin-2-yl]-1-methyl-pyrazole-4-sulfonamide

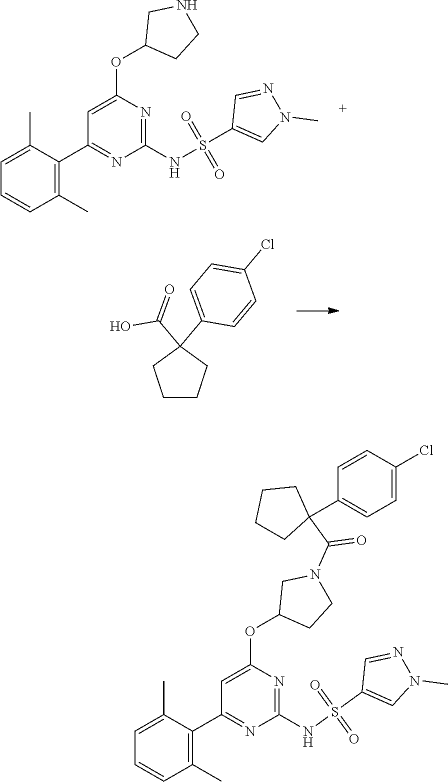 [SMILES: Cc1cccc(C)c1-c1cc(OC2CCNC2)nc(NS(=O)(=O)c2cnn(C)c2)n1 ; O=C(O)C1(c2ccc(Cl)cc2)CCCC1 ; Cc1cccc(C)c1-c1cc(OC2CCN(C(=O)C3(c4ccc(Cl)cc4)CCCC3)C2)nc(NS(=O)(=O)c2cnn(C)c2)n1]

N-[4-(2,6-dimethylphenyl)-6-pyrrolidin-3-yloxy-pyrimidin-2-yl]-1-methyl-pyrazole-4-sulfonamide (30 mg, 0.07001 mmol) in DMF (0.2 mL) was added to 1-(4-chlorophenyl)cyclopentanecarboxylic acid (approximately 23.59 mg, 0.1050 mmol), followed by HATU (approximately 39.92 mg, 0.1050 mmol), and K₂CO₃ (approximately 38.70 mg, 0.2800 mmol). The reaction mixture was shaken for 16 hours at 70° C. The reaction mixture was filtered and purified by reverse phase HPLC to give N-[4-[1-[1-(4-chlorophenyl)cyclopentanecarbonyl]pyrrolidin-3-yl]oxy-6-(2,6-dimethylphenyl)pyrimidin-2-yl]-1-methyl-pyrazole-4-sulfonamide (29.7 mg, 67%). ESI-MS m/z calc. 634.2129, found 635.32 (M+1)⁺; Retention time: 1.85 minutes; LC method A. ¹H NMR (400 MHz, DMSO-d₆) δ 11.59 (s, 1H), 8.29-8.21 (m, 1H), 7.76-7.70 (m, 1H), 7.43-7.37 (m, 1H), 7.30-7.27 (m, 1H), 7.24 -7.22 (m, 1H), 7.19-7.16 (m, 1H), 7.14-7.11 (m, 2H), 6.37-6.19 (m, 1H), 5.37-5.21 (m, 1H), 3.85 (d, J=6.2 Hz, 3H), 3.75-3.69 (m, 1H), 3.23-3.18 (m, 1H), 3.09-2.88 (m, 1H), 2.37-2.21 (m, 3H), 2.01 (s, 6H), 1.97-1.86 (m, 3H), 1.76-1.56 (m, 6H).

### Example 259: Preparation of Compound 728

### Step 1: N-[4-(2,6-Dimethylphenyl)-6-[1-[1-(4-fluorophenyl)cyclopentanecarbonyl]pyrrolidin-3-yl]oxy-pyrimidin-2-yl]-1-methyl-pyrazole-4-sulfonamide

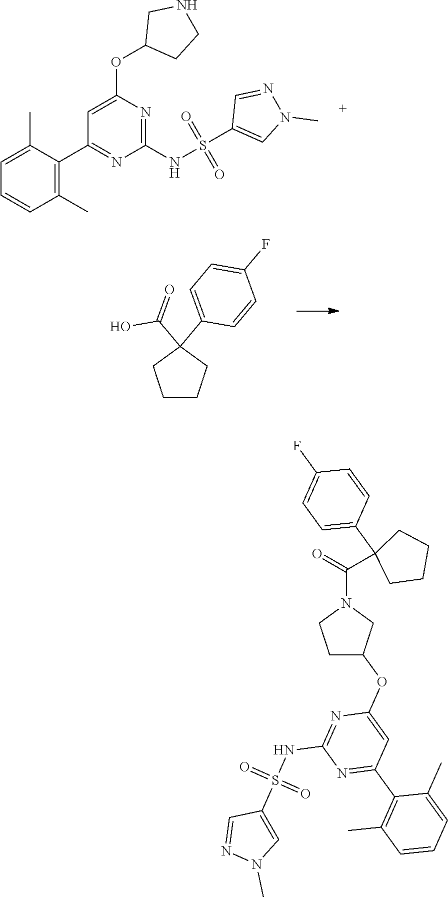 [SMILES: Cc1cccc(C)c1-c1cc(OC2CCNC2)nc(NS(=O)(=O)c2cnn(C)c2)n1 ; O=C(O)C1(c2ccc(F)cc2)CCCC1 ; Cc1cccc(C)c1-c1cc(OC2CCN(C(=O)C3(c4ccc(F)cc4)CCCC3)C2)nc(NS(=O)(=O)c2cnn(C)c2)n1]

N-[4-(2,6-dimethylphenyl)-6-pyrrolidin-3-yloxy-pyrimidin-2-yl]-1-methyl-pyrazole-4-sulfonamide (30 mg, 0.07001 mmol) in DMF (0.2 mL) was added to 1-(4-fluorophenyl)cyclopentanecarboxylic acid (approximately 21.86 mg, 0.1050 mmol) followed by HATU (approximately 39.92 mg, 0.1050 mmol), and K₂CO₃ (approximately 38.70 mg, 0.2800 mmol). The reaction mixture was shaken for 16 hours at 70° C. The reaction mixture was filtered and purified by reverse phase HPLC to give N-[4-(2,6-dimethylphenyl)-6-[1-[1-(4-fluorophenyl)cyclopentanecarbonyl]pyrrolidin-3-yl]oxy-pyrimidin-2-yl]-1-methyl-pyrazole-4-sulfonamide (29.8 mg, 69%). ESI-MS m/z calc. 618.24243, found 619.35 (M+1)⁺; Retention time: 1.72 minutes; LC method A. ¹H NMR (400 MHz, DMSO-d₆) δ 11.57 (s, 1H), 8.26 (s, 1H), 7.73 (s, 1H), 7.24 (t, J=7.3 Hz, 2H), 7.20-7.08 (m, 4H), 7.07-6.94 (m, 1H), 6.50-6.11 (m, 1H), 5.40-5.15 (m, 1H), 3.85 (s, 3H), 3.79-3.63 (m, 1H), 3.56-3.47 (m, 2H), 3.24 (s, 1H), 3.13-2.85 (m, 1H), 2.41-2.26 (m, 2H), 2.26-2.16 (m, 1H), 2.08 (s, 2H), 2.05-1.93 (m, 6H), 1.75-1.61 (m, 3H), 1.60-1.47 (m, 1H).

### Example 260: Preparation of Compound 729

### Step 1: N-[4-(2,6-Dimethylphenyl)-6-[1-[1-(p-tolyl)cyclopropanecarbonyl]pyrrolidin-3-yl]oxy-pyrimidin-2-yl]-1-methyl-pyrazole-4-sulfonamide

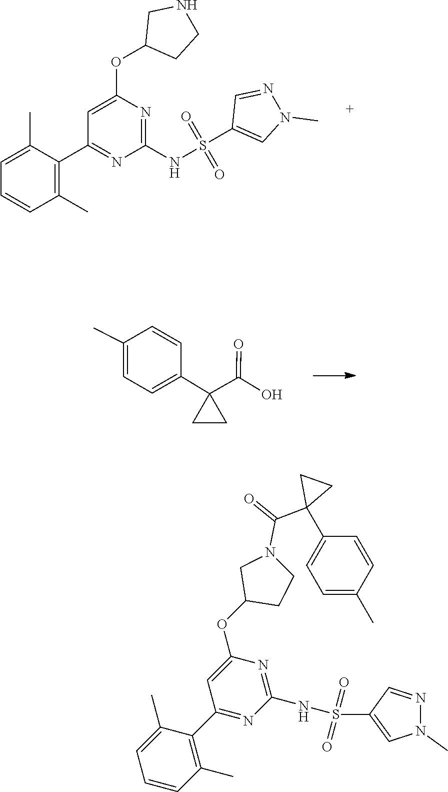

N-[4-(2,6-Dimethylphenyl)-6-pyrrolidin-3-yloxy-pyrimidin-2-yl]-1-methyl-pyrazole-4-sulfonamide (30 mg, 0.07001 mmol) in DMF (0.2 mL) was added to 1-(p-tolyl)cyclopropanecarboxylic acid (approximately 18.50 mg, 0.1050 mmol), followed by HATU (approximately 39.92 mg, 0.1050 mmol), and K₂CO₃ (approximately 38.70 mg, 0.2800 mmol). The reaction mixture was shaken for 16 hours at 70° C. The reaction mixture was filtered and purified by reverse phase HPLC to give N-[4-(2,6-dimethylphenyl)-6-[1-[1-(p-tolyl)cyclopropanecarbonyl]pyrrolidin-3-yl]oxy-pyrimidin-2-yl]-1-methyl-pyrazole-4-sulfonamide (29.3 mg, 71%). ESI-MS m/z calc. 586.2362, found 587.35 (M+1)⁺; Retention time: 1.63 minutes; LC method A. ¹H NMR (400 MHz, DMSO-d₆) δ 11.63 (s, 1H), 8.40-8.11 (m, 1H), 7.74 (d, J=6.7 Hz, 1H), 7.24 (t, J=7.4 Hz, 1H), 7.16-7.09 (m, 3H), 7.08-7.04 (m, 1H), 7.02 (s, 1H), 6.35 (s, 1H), 5.47-5.29 (m, 1H), 3.91-3.78 (m, 3H), 3.75-3.64 (m, 1H), 3.60-3.47 (m, 3H), 3.24-3.10 (m, 1H), 2.29-2.10 (m, 4H), 2.09-1.81 (m, 7H), 1.39-1.27 (m, 1H), 1.28-1.14 (m, 1H), 1.13-0.95 (m, 2H).

### Example 261: Preparation of Compound 730

### Step 1: N-[4-(2,6-Dimethylphenyl)-6-[1-[1-(2-fluorophenyl)cyclohexanecarbonyl]pyrrolidin-3-yl]oxy-pyrimidin-2-yl]-1-methyl-pyrazole-4-sulfonamide

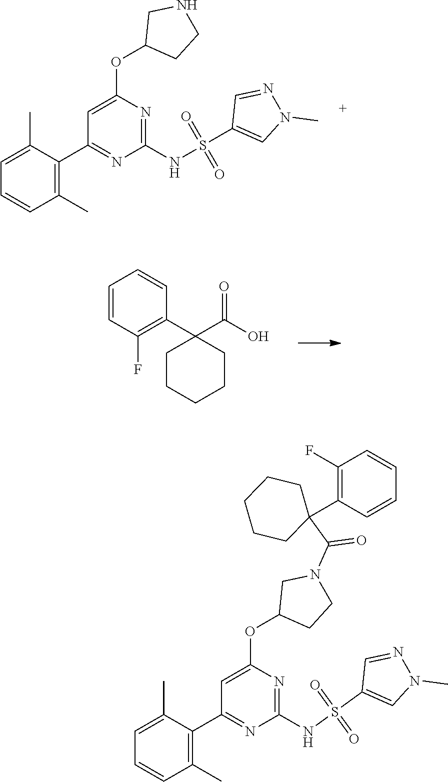 [SMILES: Cc1cccc(C)c1-c1cc(OC2CCNC2)nc(NS(=O)(=O)c2cnn(C)c2)n1 ; O=C(O)C1(c2ccccc2F)CCCCC1 ; Cc1cccc(C)c1-c1cc(OC2CCN(C(=O)C3(c4ccccc4F)CCCCC3)C2)nc(NS(=O)(=O)c2cnn(C)c2)n1]

N-[4-(2,6-Dimethylphenyl)-6-pyrrolidin-3-yloxy-pyrimidin-2-yl]-1-methyl-pyrazole-4-sulfonamide (30 mg, 0.07001 mmol) in DMF (0.2 mL) was added to 1-(2-fluorophenyl)cyclohexanecarboxylic acid (approximately 23.34 mg, 0.1050 mmol), followed by HATU (approximately 39.92 mg, 0.1050 mmol), and K₂CO₃ (approximately 38.70 mg, 0.2800 mmol). The reaction mixture was shaken for 16 hours at 70° C. The reaction mixture was filtered and purified by reverse phase HPLC to give N-[4-(2,6-dimethylphenyl)-6-[1-[1-(2-fluorophenyl)cyclohexanecarbonyl]pyrrolidin-3-yl]oxy-pyrimidin-2-yl]-1-methyl-pyrazole-4-sulfonamide (22.4 mg, 51% ). ESI-MS m/z calc. 632.2581, found 633.39 (M+1)⁺; Retention time: 1.78 minutes; LC method A. ¹H NMR (400 MHz, DMSO-d₆) δ 11.51 (s, 1H), 8.43-8.17 (m, 1H), 7.73 (d, J=4.9 Hz, 1H), 7.60-7.28 (m, 2H), 7.24 (t, J=7.6 Hz, 1H), 7.20-7.09 (m, 2H), 7.09-6.88 (m, 1H), 6.46-6.10 (m, 1H), 5.44-5.09 (m, 1H), 3.85 (s, 3H), 3.73 (s, 3H), 3.21-2.90 (m, 2H), 2.25 (d, J=21.0 Hz, 3H), 2.02 (s, 6H), 1.95-1.66 (m, 4H), 1.65-1.13 (in, 5H).

### Example 262: Characterization of Compounds 731-879

The compounds in the following tables were prepared in a manner analogous to that described above using commercially available reagents and intermediates described herein.

| | | LCMS | | | |
|---|---|---|---|---|---|
| | | Room | | | |
| Compound | | temperature | Calc. | | LCMS |
| Number | Structure | (min) | mass | M + 1 | Method |
| 731 | 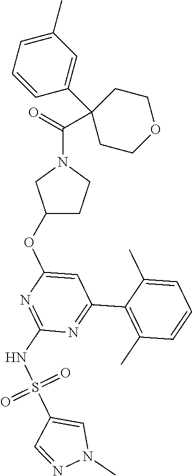 [SMILES: Cc1cccc(C2(C(=O)N3CCC(Oc4cc(-c5c(C)cccc5C)nc(NS(=O)(=O)c5cnn(C)c5)n4)C3)CCOCC2)c1] | 1.58 | 630.262 | 631.4 | A |
| 732 | 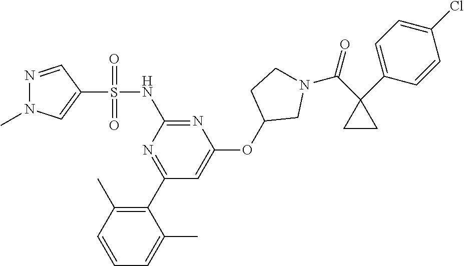 [SMILES: Cc1cccc(C)c1-c1cc(OC2CCN(C(=O)C3(c4ccc(Cl)cc4)CC3)C2)nc(NS(=O)(=O)c2cnn(C)c2)n1] | 1.67 | 606.182 | 607.31 | A |
| 733 | 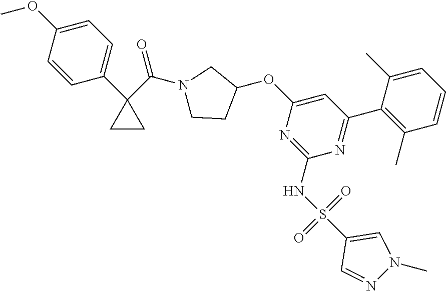 [SMILES: COc1ccc(C2(C(=O)N3CCC(Oc4cc(-c5c(C)cccc5C)nc(NS(=O)(=O)c5cnn(C)c5)n4)C3)CC2)cc1] | 1.55 | 602.231 | 603.39 | A |
| 734 | 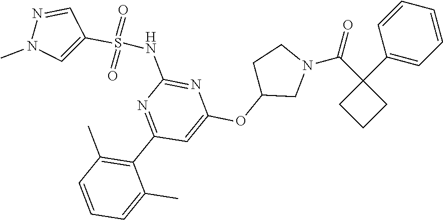 [SMILES: Cc1cccc(C)c1-c1cc(OC2CCN(C(=O)C3(c4ccccc4)CCC3)C2)nc(NS(=O)(=O)c2cnn(C)c2)n1] | 1.63 | 586.236 | 587.35 | A |
| 735 | 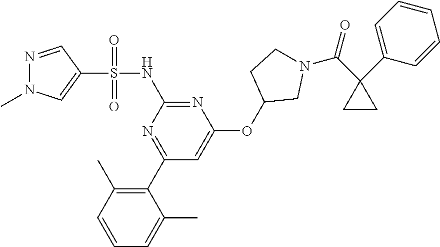 [SMILES: Cc1cccc(C)c1-c1cc(OC2CCN(C(=O)C3(c4ccccc4)CC3)C2)nc(NS(=O)(=O)c2cnn(C)c2)n1] | 1.57 | 572.221 | 573.35 | A |
| 736 | 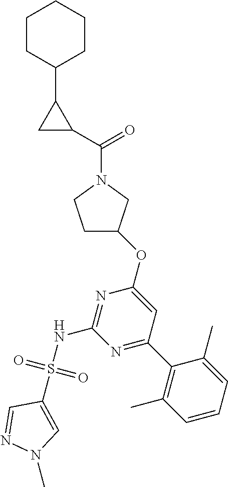 [SMILES: Cc1cccc(C)c1-c1cc(OC2CCN(C(=O)C3CC3C3CCCCC3)C2)nc(NS(=O)(=O)c2cnn(C)c2)n1] | 1.8 | 578.268 | 579.4 | A |
| 737 | 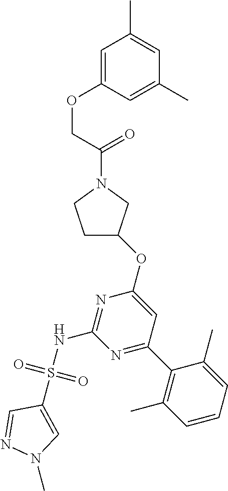 [SMILES: Cc1cc(C)cc(OCC(=O)N2CCC(Oc3cc(-c4c(C)cccc4C)nc(NS(=O)(=O)c4cnn(C)c4)n3)C2)c1] | 1.67 | 590.231 | 591.38 | A |
| 738 | 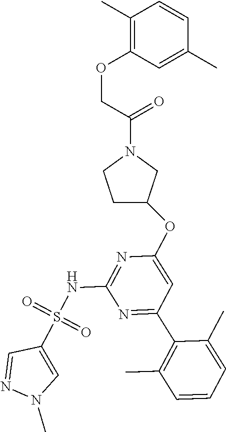 [SMILES: Cc1ccc(C)c(OCC(=O)N2CCC(Oc3cc(-c4c(C)cccc4C)nc(NS(=O)(=O)c4cnn(C)c4)n3)C2)c1] | 1.68 | 590.231 | 591.34 | A |
| 739 | 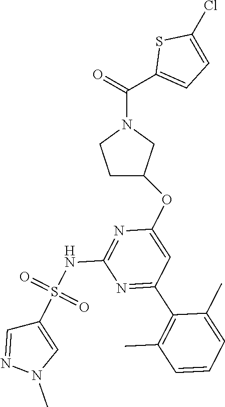 [SMILES: Cc1cccc(C)c1-c1cc(OC2CCN(C(=O)c3ccc(Cl)s3)C2)nc(NS(=O)(=O)c2cnn(C)c2)n1] | 1.62 | 572.107 | 573.24 | A |
| 740 | 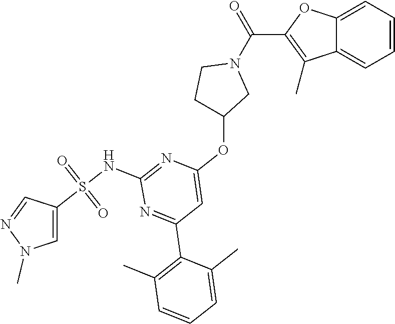 [SMILES: Cc1cccc(C)c1-c1cc(OC2CCN(C(=O)c3oc4ccccc4c3C)C2)nc(NS(=O)(=O)c2cnn(C)c2)n1] | 1.72 | 586.2 | 587.35 | A |
| 741 | 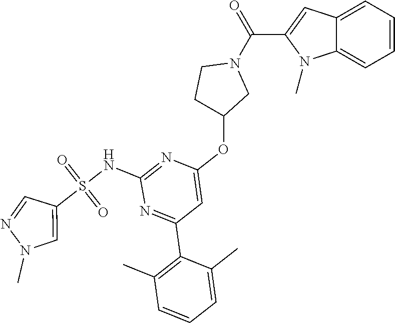 [SMILES: Cc1cccc(C)c1-c1cc(OC2CCN(C(=O)c3cc4ccccc4n3C)C2)nc(NS(=O)(=O)c2cnn(C)c2)n1] | 1.65 | 585.216 | 586.34 | A |
| 742 | 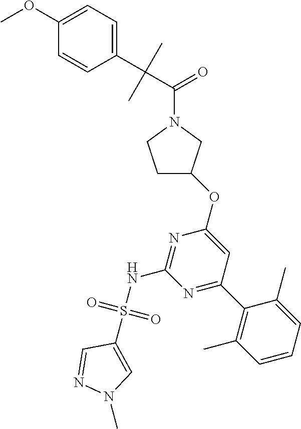 [SMILES: COc1ccc(C(C)(C)C(=O)N2CCC(Oc3cc(-c4c(C)cccc4C)nc(NS(=O)(=O)c4cnn(C)c4)n3)C2)cc1] | 1.62 | 604.247 | 605.38 | A |
| 743 | 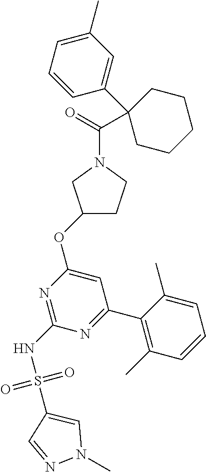 [SMILES: Cc1cccc(C2(C(=O)N3CCC(Oc4cc(-c5c(C)cccc5C)nc(NS(=O)(=O)c5cnn(C)c5)n4)C3)CCCCC2)c1] | 1.9 | 628.283 | 629.4 | A |
| 744 | 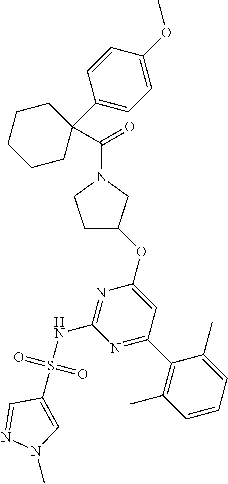 [SMILES: COc1ccc(C2(C(=O)N3CCC(Oc4cc(-c5c(C)cccc5C)nc(NS(=O)(=O)c5cnn(C)c5)n4)C3)CCCCC2)cc1] | 1.82 | 644.278 | 645.4 | A |
| 745 | 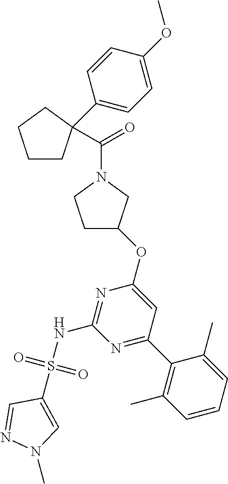 [SMILES: COc1ccc(C2(C(=O)N3CCC(Oc4cc(-c5c(C)cccc5C)nc(NS(=O)(=O)c5cnn(C)c5)n4)C3)CCCC2)cc1] | 1.72 | 630.262 | 631.4 | A |
| 746 | 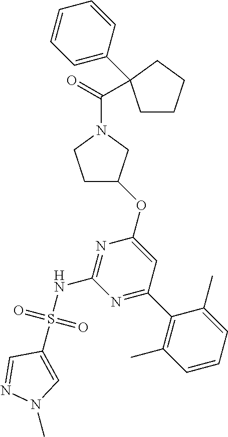 [SMILES: Cc1cccc(C)c1-c1cc(OC2CCN(C(=O)C3(c4ccccc4)CCCC3)C2)nc(NS(=O)(=O)c2cnn(C)c2)n1] | 1.72 | 600.252 | 601.39 | A |
| 747 | 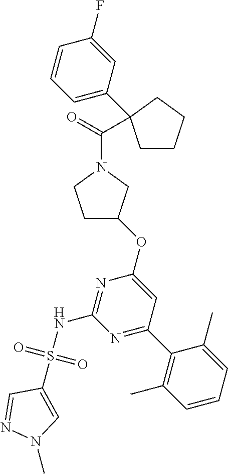 [SMILES: Cc1cccc(C)c1-c1cc(OC2CCN(C(=O)C3(c4cccc(F)c4)CCCC3)C2)nc(NS(=O)(=O)c2cnn(C)c2)n1] | 1.75 | 618.242 | 619.39 | A |
| 748 | 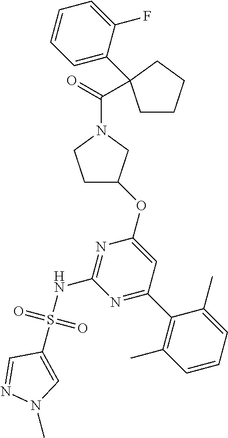 [SMILES: Cc1cccc(C)c1-c1cc(OC2CCN(C(=O)C3(c4ccccc4F)CCCC3)C2)nc(NS(=O)(=O)c2cnn(C)c2)n1] | 1.76 | 618.242 | 619 | A |
| 749 | 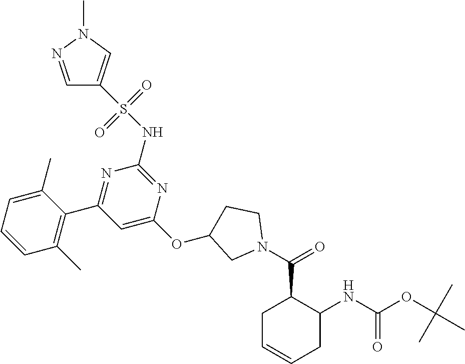 [SMILES: Cc1cccc(C)c1-c1cc(OC2CCN(C(=O)[C@@H]3CC=CCC3NC(=O)OC(C)(C)C)C2)nc(NS(=O)(=O)c2cnn(C)c2)n1] | 1.17 | 651.284 | 642.43 | R |
| 750 | 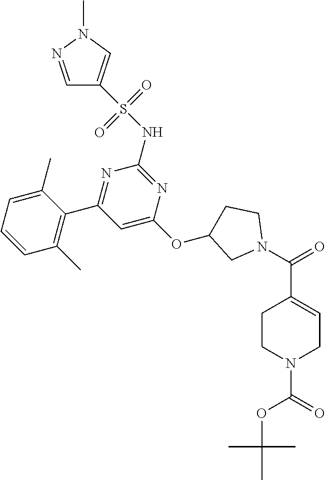 [SMILES: Cc1cccc(C)c1-c1cc(OC2CCN(C(=O)C3=CCN(C(=O)OC(C)(C)C)CC3)C2)nc(NS(=O)(=O)c2cnn(C)c2)n1] | 1.16 | 637.268 | 638.43 | R |
| 751 | 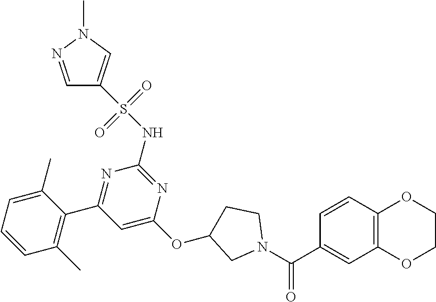 [SMILES: Cc1cccc(C)c1-c1cc(OC2CCN(C(=O)c3ccc4c(c3)OCCO4)C2)nc(NS(=O)(=O)c2cnn(C)c2)n1] | 1.04 | 590.195 | 591.35 | R |
| 752 | 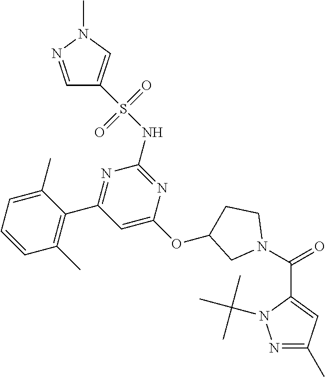 [SMILES: Cc1cc(C(=O)N2CCC(Oc3cc(-c4c(C)cccc4C)nc(NS(=O)(=O)c4cnn(C)c4)n3)C2)n(C(C)(C)C)n1] | 1.12 | 592.258 | 593.42 | R |
| 753 | 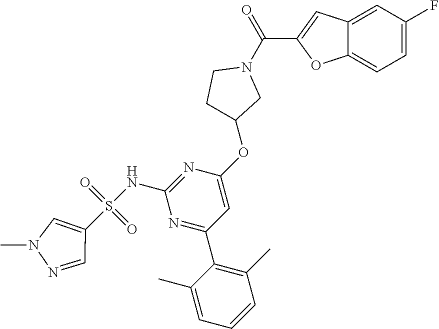 [SMILES: Cc1cccc(C)c1-c1cc(OC2CCN(C(=O)c3cc4cc(F)ccc4o3)C2)nc(NS(=O)(=O)c2cnn(C)c2)n1] | 1.2 | 590.175 | 591.32 | R |
| 754 | 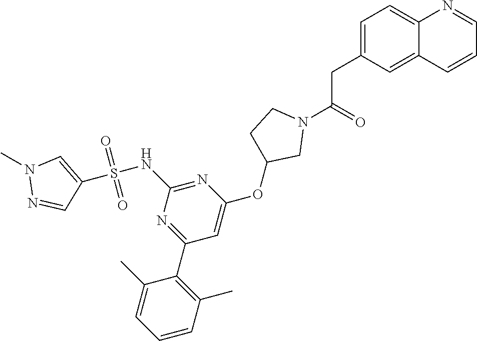 [SMILES: Cc1cccc(C)c1-c1cc(OC2CCN(C(=O)Cc3ccc4ncccc4c3)C2)nc(NS(=O)(=O)c2cnn(C)c2)n1] | 0.76 | 597.216 | 598.39 | R |
| 755 | 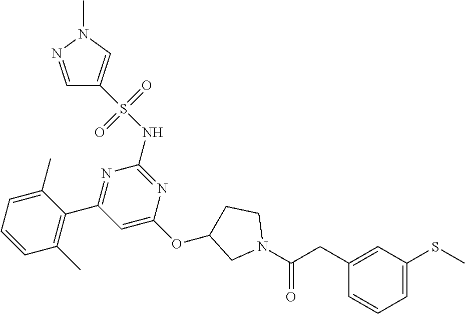 [SMILES: CSc1cccc(CC(=O)N2CCC(Oc3cc(-c4c(C)cccc4C)nc(NS(=O)(=O)c4cnn(C)c4)n3)C2)c1] | 1.16 | 592.193 | 593.35 | R |
| 756 | 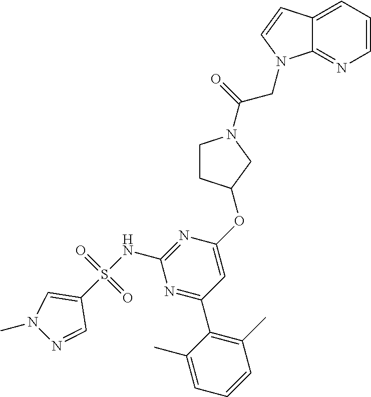 [SMILES: Cc1cccc(C)c1-c1cc(OC2CCN(C(=O)Cn3ccc4cccnc43)C2)nc(NS(=O)(=O)c2cnn(C)c2)n1] | 0.92 | 586.211 | 587.36 | R |
| 757 | 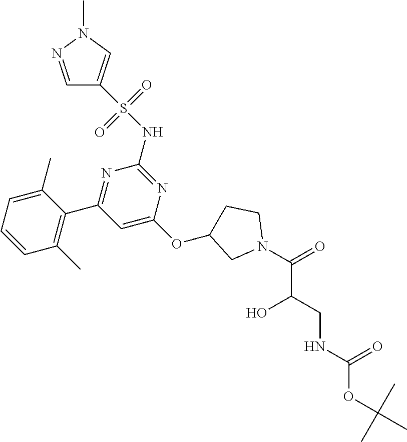 [SMILES: Cc1cccc(C)c1-c1cc(OC2CCN(C(=O)C(O)CNC(=O)OC(C)(C)C)C2)nc(NS(=O)(=O)c2cnn(C)c2)n1] | 1.01 | 615.248 | 616.42 | R |
| 758 | 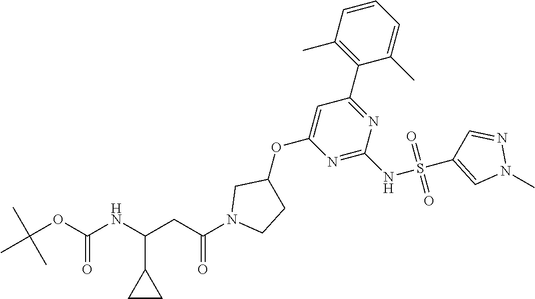 [SMILES: Cc1cccc(C)c1-c1cc(OC2CCN(C(=O)CC(NC(=O)OC(C)(C)C)C3CC3)C2)nc(NS(=O)(=O)c2cnn(C)c2)n1] | 1.17 | 639.284 | 640.46 | R |
| 759 | 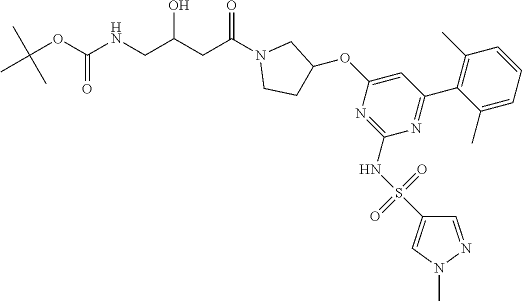 [SMILES: Cc1cccc(C)c1-c1cc(OC2CCN(C(=O)CC(O)CNC(=O)OC(C)(C)C)C2)nc(NS(=O)(=O)c2cnn(C)c2)n1] | 1 | 629.263 | 630.42 | R |
| 760 | 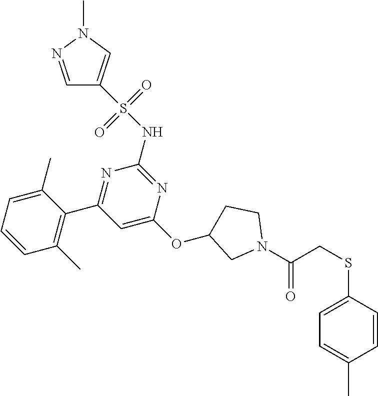 [SMILES: Cc1ccc(SCC(=O)N2CCC(Oc3cc(-c4c(C)cccc4C)nc(NS(=O)(=O)c4cnn(C)c4)n3)C2)cc1] | 1.2 | 592.193 | 593.35 | R |
| 761 | 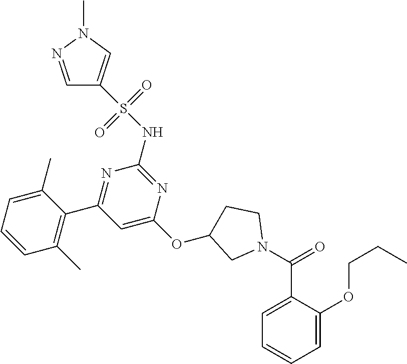 [SMILES: CCCOc1ccccc1C(=O)N1CCC(Oc2cc(-c3c(C)cccc3C)nc(NS(=O)(=O)c3cnn(C)c3)n2)C1] | 1.19 | 590.231 | 591.39 | R |
| 762 | 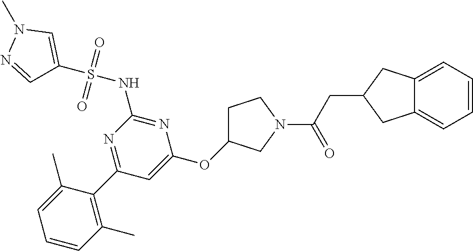 [SMILES: Cc1cccc(C)c1-c1cc(OC2CCN(C(=O)CC3Cc4ccccc4C3)C2)nc(NS(=O)(=O)c2cnn(C)c2)n1] | 1.23 | 586.236 | 587.43 | R |
| 763 | 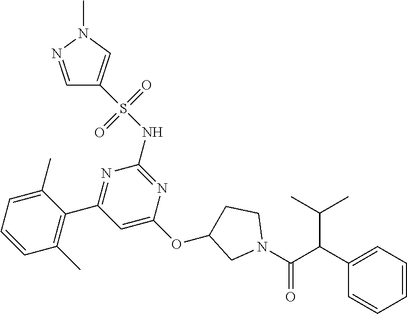 [SMILES: Cc1cccc(C)c1-c1cc(OC2CCN(C(=O)C(c3ccccc3)C(C)C)C2)nc(NS(=O)(=O)c2cnn(C)c2)n1] | 1.29 | 588.252 | 589.43 | R |
| 764 | 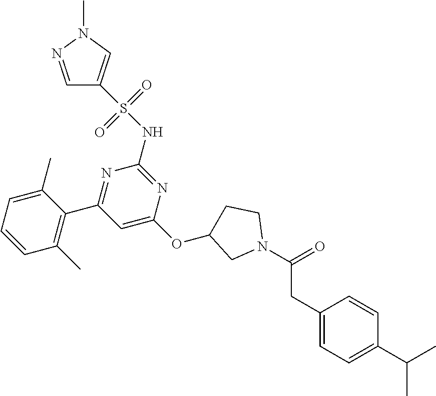 [SMILES: Cc1cccc(C)c1-c1cc(OC2CCN(C(=O)Cc3ccc(C(C)C)cc3)C2)nc(NS(=O)(=O)c2cnn(C)c2)n1] | 1.28 | 588.252 | 589.43 | R |
| 765 | 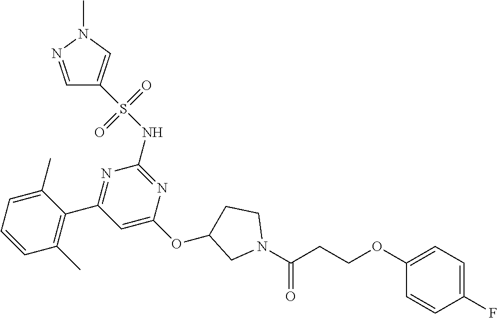 [SMILES: Cc1cccc(C)c1-c1cc(OC2CCN(C(=O)CCOc3ccc(F)cc3)C2)nc(NS(=O)(=O)c2cnn(C)c2)n1] | 1.14 | 594.206 | 595.38 | R |
| 766 | 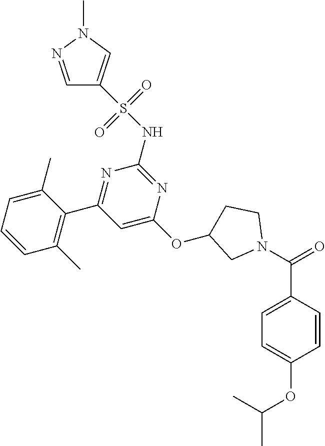 [SMILES: Cc1cccc(C)c1-c1cc(OC2CCN(C(=O)c3ccc(OC(C)C)cc3)C2)nc(NS(=O)(=O)c2cnn(C)c2)n1] | 1.2 | 590.231 | 591.39 | R |
| 767 | 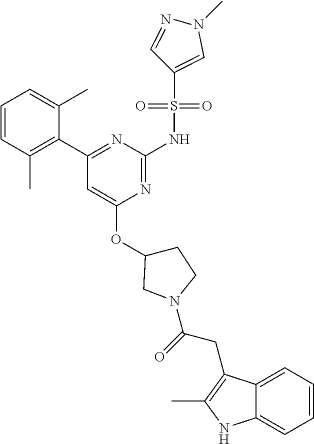 [SMILES: Cc1cccc(C)c1-c1cc(OC2CCN(C(=O)Cc3c(C)[nH]c4ccccc34)C2)nc(NS(=O)(=O)c2cnn(C)c2)n1] | 1.08 | 599.231 | 600.42 | R |
| 768 | 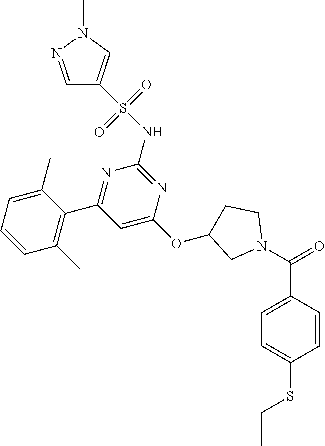 [SMILES: CCSc1ccc(C(=O)N2CCC(Oc3cc(-c4c(C)cccc4C)nc(NS(=O)(=O)c4cnn(C)c4)n3)C2)cc1] | 1.22 | 592.193 | 593.55 | R |
| 769 | 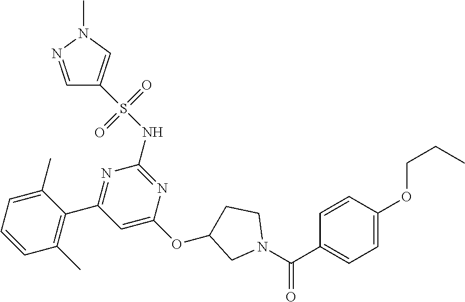 [SMILES: CCCOc1ccc(C(=O)N2CCC(Oc3cc(-c4c(C)cccc4C)nc(NS(=O)(=O)c4cnn(C)c4)n3)C2)cc1] | 1.23 | 590.231 | 591.39 | R |
| 770 | 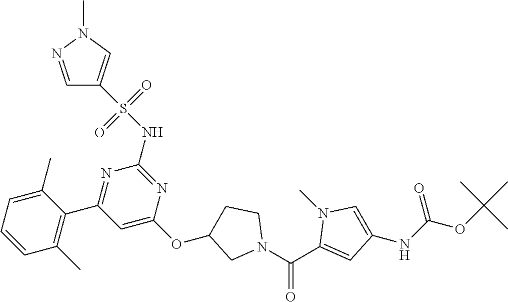 [SMILES: Cc1cccc(C)c1-c1cc(OC2CCN(C(=O)c3cc(NC(=O)OC(C)(C)C)cn3C)C2)nc(NS(=O)(=O)c2cnn(C)c2)n1] | 1.15 | 650.264 | 651.42 | R |
| 771 | 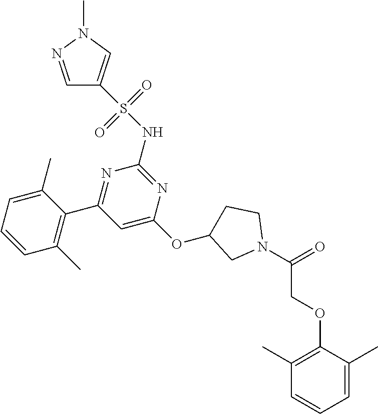 [SMILES: Cc1cccc(C)c1OCC(=O)N1CCC(Oc2cc(-c3c(C)cccc3C)nc(NS(=O)(=O)c3cnn(C)c3)n2)C1] | 1.19 | 590.231 | 591.42 | R |
| 772 | 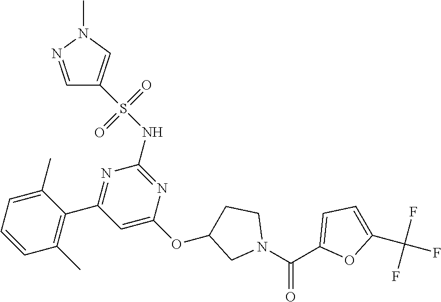 [SMILES: Cc1cccc(C)c1-c1cc(OC2CCN(C(=O)c3ccc(C(F)(F)F)o3)C2)nc(NS(=O)(=O)c2cnn(C)c2)n1] | 1.18 | 590.156 | 591.32 | R |
| 773 | 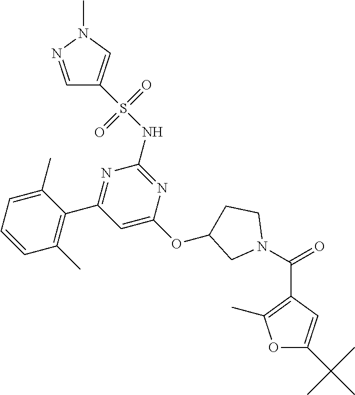 [SMILES: Cc1cccc(C)c1-c1cc(OC2CCN(C(=O)c3cc(C(C)(C)C)oc3C)C2)nc(NS(=O)(=O)c2cnn(C)c2)n1] | 1.33 | 592.247 | 593.42 | R |
| 774 | 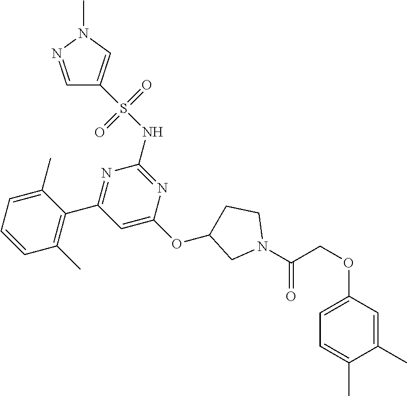 [SMILES: Cc1ccc(OCC(=O)N2CCC(Oc3cc(-c4c(C)cccc4C)nc(NS(=O)(=O)c4cnn(C)c4)n3)C2)cc1C] | 1.21 | 590.231 | 591.39 | R |
| 775 | 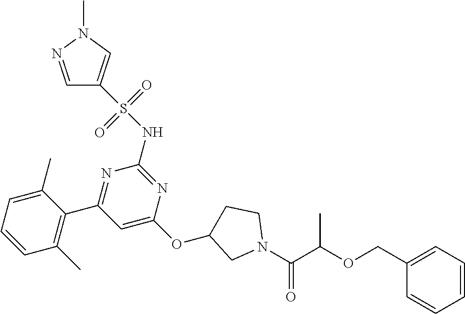 [SMILES: Cc1cccc(C)c1-c1cc(OC2CCN(C(=O)C(C)OCc3ccccc3)C2)nc(NS(=O)(=O)c2cnn(C)c2)n1] | 1.13 | 590.231 | 591.39 | R |
| 776 | 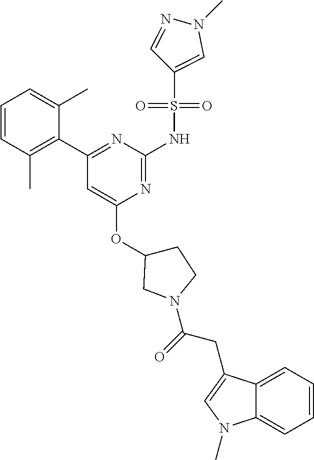 [SMILES: Cc1cccc(C)c1-c1cc(OC2CCN(C(=O)Cc3cn(C)c4ccccc34)C2)nc(NS(=O)(=O)c2cnn(C)c2)n1] | 1.15 | 599.231 | 600.38 | A |
| 777 | 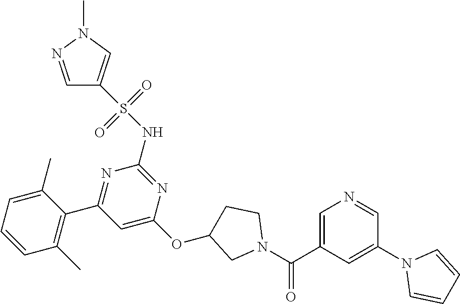 [SMILES: Cc1cccc(C)c1-c1cc(OC2CCN(C(=O)c3cncc(-n4cccc4)c3)C2)nc(NS(=O)(=O)c2cnn(C)c2)n1] | 1.05 | 598.211 | 599.37 | R |
| 778 | 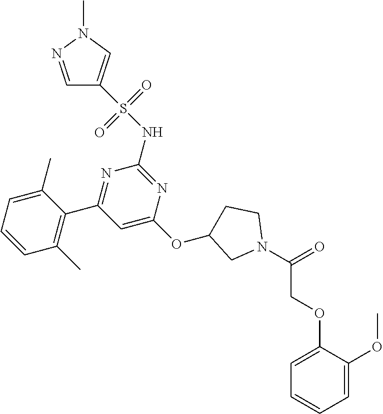 [SMILES: COc1ccccc1OCC(=O)N1CCC(Oc2cc(-c3c(C)cccc3C)nc(NS(=O)(=O)c3cnn(C)c3)n2)C1] | 1.08 | 592.21 | 593.38 | R |
| 779 | 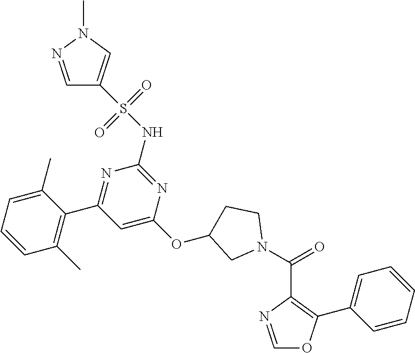 [SMILES: Cc1cccc(C)c1-c1cc(OC2CCN(C(=O)c3ncoc3-c3ccccc3)C2)nc(NS(=O)(=O)c2cnn(C)c2)n1] | 1.12 | 599.195 | 600.39 | R |
| 780 | 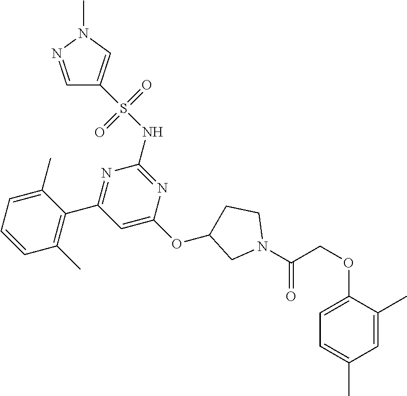 [SMILES: Cc1ccc(OCC(=O)N2CCC(Oc3cc(-c4c(C)cccc4C)nc(NS(=O)(=O)c4cnn(C)c4)n3)C2)c(C)c1] | 1.24 | 590.231 | 591.39 | R |
| 781 | 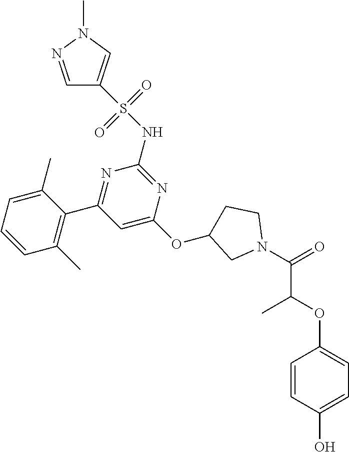 [SMILES: Cc1cccc(C)c1-c1cc(OC2CCN(C(=O)C(C)Oc3ccc(O)cc3)C2)nc(NS(=O)(=O)c2cnn(C)c2)n1] | 0.94 | 592.21 | 593.38 | R |
| 782 | 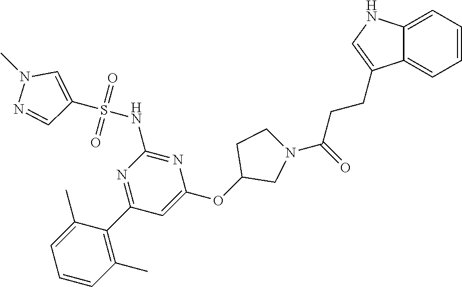 [SMILES: Cc1cccc(C)c1-c1cc(OC2CCN(C(=O)CCc3c[nH]c4ccccc34)C2)nc(NS(=O)(=O)c2cnn(C)c2)n1] | 1.1 | 599.231 | 600.38 | A |
| 783 | 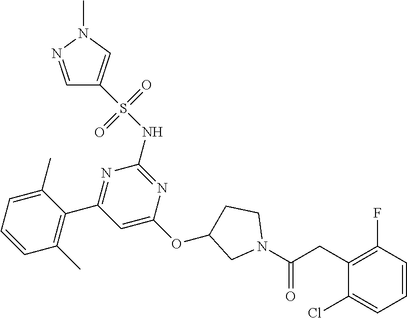 [SMILES: Cc1cccc(C)c1-c1cc(OC2CCN(C(=O)Cc3c(F)cccc3Cl)C2)nc(NS(=O)(=O)c2cnn(C)c2)n1] | 1.18 | 598.157 | 599.33 | R |
| 784 | 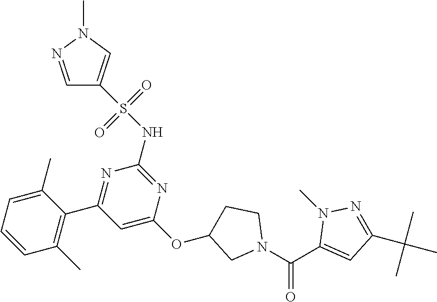 [SMILES: Cc1cccc(C)c1-c1cc(OC2CCN(C(=O)c3cc(C(C)(C)C)nn3C)C2)nc(NS(=O)(=O)c2cnn(C)c2)n1] | 1.19 | 592.258 | 593.42 | R |
| 785 | 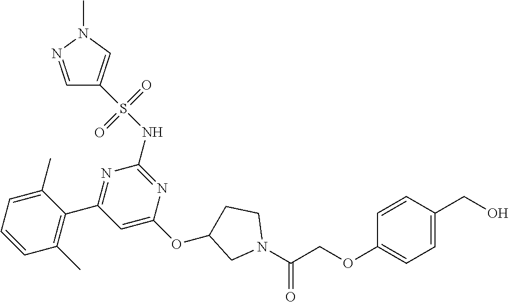 [SMILES: Cc1cccc(C)c1-c1cc(OC2CCN(C(=O)COc3ccc(CO)cc3)C2)nc(NS(=O)(=O)c2cnn(C)c2)n1] | 0.91 | 592.21 | 593.38 | R |
| 786 | 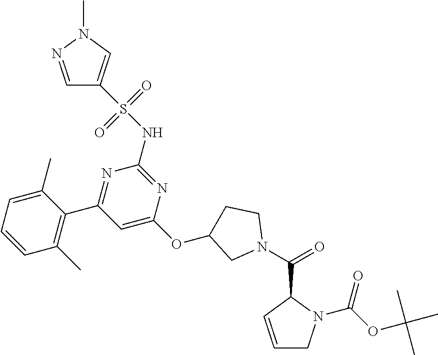 [SMILES: Cc1cccc(C)c1-c1cc(OC2CCN(C(=O)[C@@H]3C=CCN3C(=O)OC(C)(C)C)C2)nc(NS(=O)(=O)c2cnn(C)c2)n1] | 1.08 | 623.253 | 624.43 | R |
| 787 | 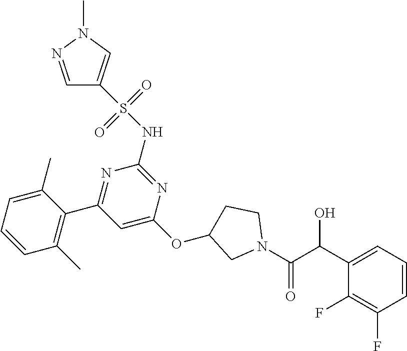 [SMILES: Cc1cccc(C)c1-c1cc(OC2CCN(C(=O)C(O)c3cccc(F)c3F)C2)nc(NS(=O)(=O)c2cnn(C)c2)n1] | 1.41 | 598.181 | 599 | A |
| 788 | 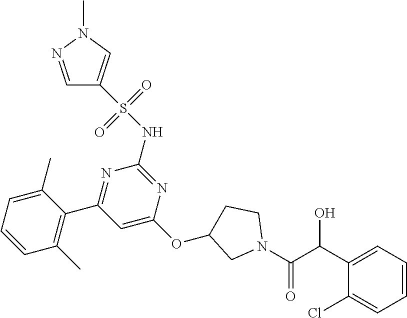 [SMILES: Cc1cccc(C)c1-c1cc(OC2CCN(C(=O)C(O)c3ccccc3Cl)C2)nc(NS(=O)(=O)c2cnn(C)c2)n1] | 1.43 | 596.161 | 597 | A |
| 789 | 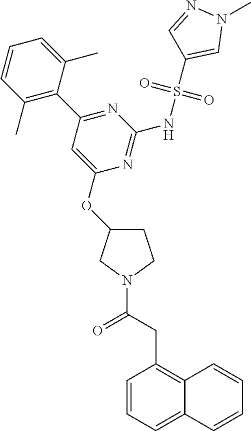 [SMILES: Cc1cccc(C)c1-c1cc(OC2CCN(C(=O)Cc3cccc4ccccc34)C2)nc(NS(=O)(=O)c2cnn(C)c2)n1] | 1.22 | 596.221 | 597.37 | R |
| 790 | 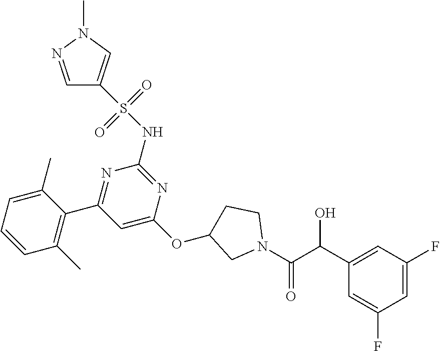 [SMILES: Cc1cccc(C)c1-c1cc(OC2CCN(C(=O)C(O)c3cc(F)cc(F)c3)C2)nc(NS(=O)(=O)c2cnn(C)c2)n1] | 1.43 | 598.181 | 599 | A |
| 791 | 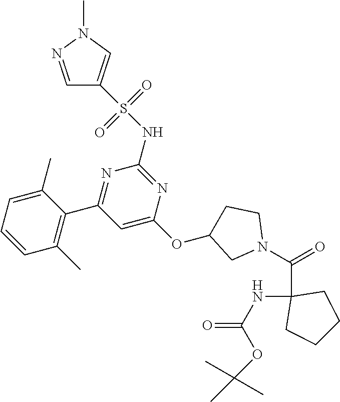 [SMILES: Cc1cccc(C)c1-c1cc(OC2CCN(C(=O)C3(NC(=O)OC(C)(C)C)CCCC3)C2)nc(NS(=O)(=O)c2cnn(C)c2)n1] | 1.15 | 639.284 | 640.49 | A |
| 792 | 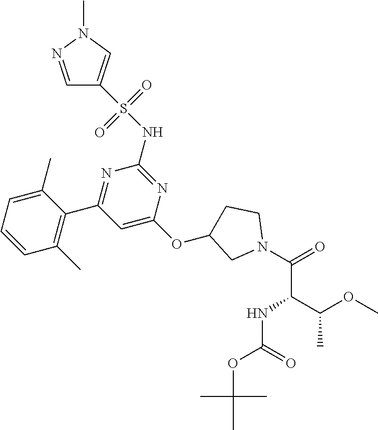 [SMILES: CO[C@H](C)[C@H](NC(=O)OC(C)(C)C)C(=O)N1CCC(Oc2cc(-c3c(C)cccc3C)nc(NS(=O)(=O)c3cnn(C)c3)n2)C1] | 1.14 | 643.279 | 644.48 | R |
| 793 | 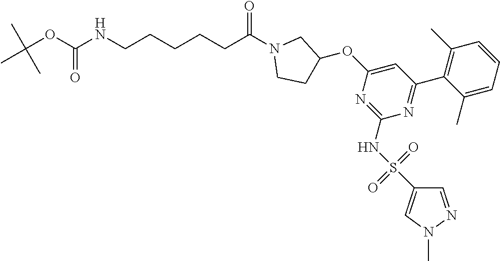 [SMILES: Cc1cccc(C)c1-c1cc(OC2CCN(C(=O)CCCCCNC(=O)OC(C)(C)C)C2)nc(NS(=O)(=O)c2cnn(C)c2)n1] | 1.16 | 641.3 | 642.49 | A |
| 794 | 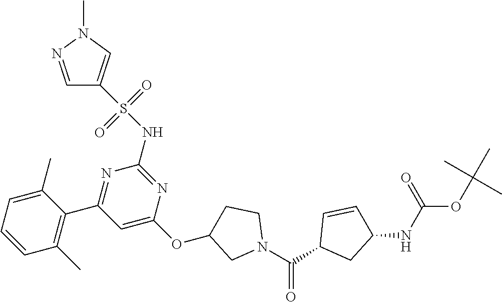 [SMILES: Cc1cccc(C)c1-c1cc(OC2CCN(C(=O)[C@@H]3C=C[C@H](NC(=O)OC(C)(C)C)C3)C2)nc(NS(=O)(=O)c2cnn(C)c2)n1] | 1.17 | 637.268 | 638.46 | R |
| 795 | 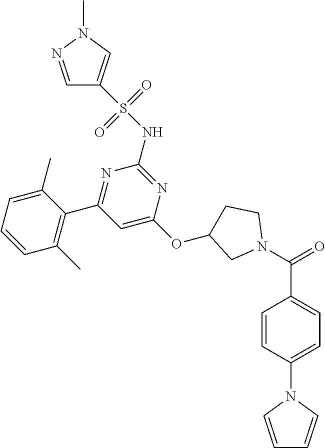 [SMILES: Cc1cccc(C)c1-c1cc(OC2CCN(C(=O)c3ccc(-n4cccc4)cc3)C2)nc(NS(=O)(=O)c2cnn(C)c2)n1] | 1.2 | 597.216 | 598.39 | R |
| 796 | 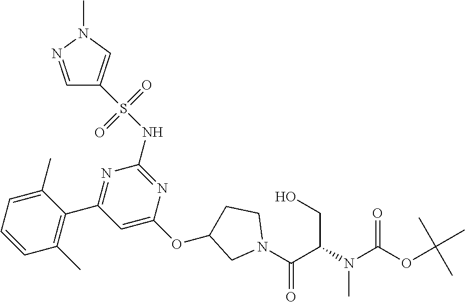 [SMILES: Cc1cccc(C)c1-c1cc(OC2CCN(C(=O)[C@H](CO)N(C)C(=O)OC(C)(C)C)C2)nc(NS(=O)(=O)c2cnn(C)c2)n1] | 1.03 | 629.263 | 630.45 | R |
| 797 | 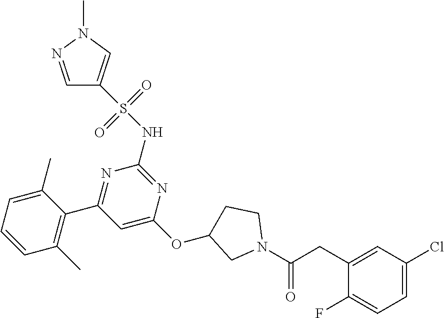 [SMILES: Cc1cccc(C)c1-c1cc(OC2CCN(C(=O)Cc3cc(Cl)ccc3F)C2)nc(NS(=O)(=O)c2cnn(C)c2)n1] | 1.19 | 598.157 | 599.33 | A |
| 798 | 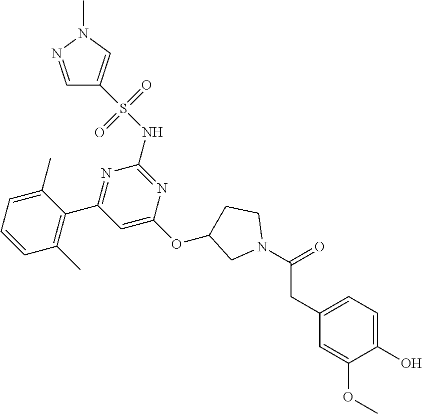 [SMILES: COc1cc(CC(=O)N2CCC(Oc3cc(-c4c(C)cccc4C)nc(NS(=O)(=O)c4cnn(C)c4)n3)C2)ccc1O] | 0.95 | 592.21 | 593.38 | A |
| 799 | 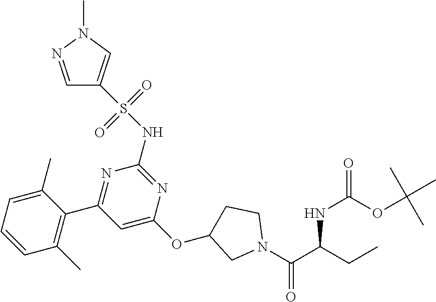 [SMILES: CC[C@H](NC(=O)OC(C)(C)C)C(=O)N1CCC(Oc2cc(-c3c(C)cccc3C)nc(NS(=O)(=O)c3cnn(C)c3)n2)C1] | 1.15 | 613.268 | 614.45 | A |
| 800 | 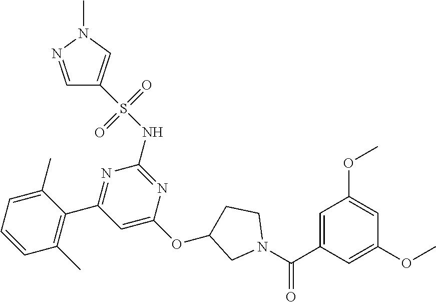 [SMILES: COc1cc(OC)cc(C(=O)N2CCC(Oc3cc(-c4c(C)cccc4C)nc(NS(=O)(=O)c4cnn(C)c4)n3)C2)c1] | 1.1 | 592.21 | 593.38 | R |
| 801 | 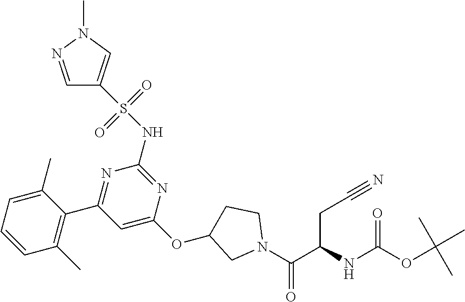 [SMILES: Cc1cccc(C)c1-c1cc(OC2CCN(C(=O)[C@@H](CC#N)NC(=O)OC(C)(C)C)C2)nc(NS(=O)(=O)c2cnn(C)c2)n1] | 1.08 | 624.248 | 625.44 | R |
| 802 | 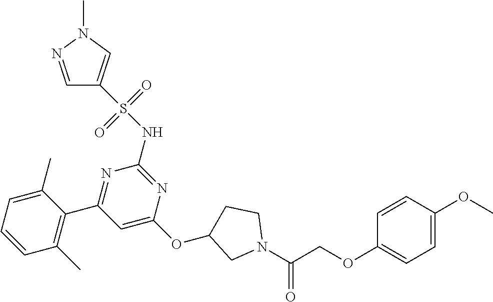 [SMILES: COc1ccc(OCC(=O)N2CCC(Oc3cc(-c4c(C)cccc4C)nc(NS(=O)(=O)c4cnn(C)c4)n3)C2)cc1] | 1.08 | 592.21 | 593.38 | R |
| 803 | 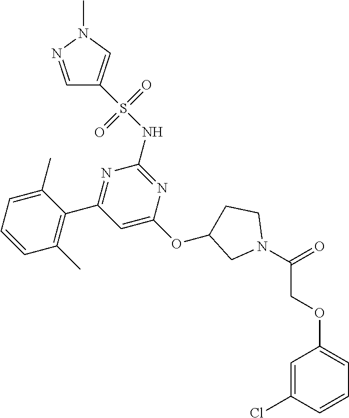 [SMILES: Cc1cccc(C)c1-c1cc(OC2CCN(C(=O)COc3cccc(Cl)c3)C2)nc(NS(=O)(=O)c2cnn(C)c2)n1] | 1.61 | 596.161 | 598 | A |
| 804 | 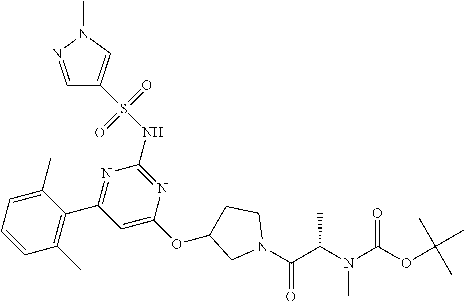 [SMILES: Cc1cccc(C)c1-c1cc(OC2CCN(C(=O)[C@H](C)N(C)C(=O)OC(C)(C)C)C2)nc(NS(=O)(=O)c2cnn(C)c2)n1] | 1.16 | 613.268 | 614.45 | R |
| 805 | 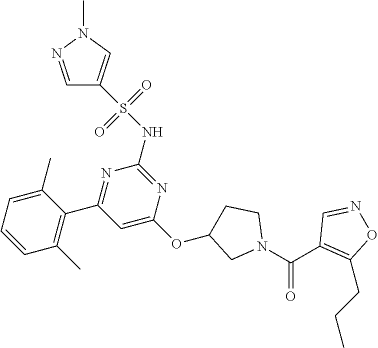 [SMILES: CCCc1oncc1C(=O)N1CCC(Oc2cc(-c3c(C)cccc3C)nc(NS(=O)(=O)c3cnn(C)c3)n2)C1] | 1.8 | 565.211 | 566.2 | A |
| 806 | 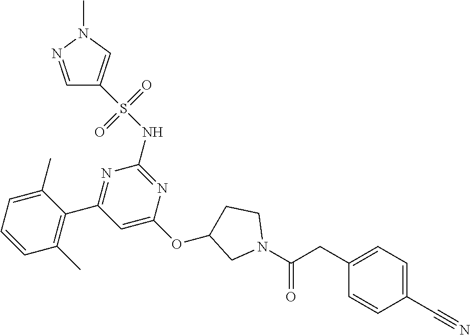 [SMILES: Cc1cccc(C)c1-c1cc(OC2CCN(C(=O)Cc3ccc(C#N)cc3)C2)nc(NS(=O)(=O)c2cnn(C)c2)n1] | 1.39 | 571.2 | 572 | A |
| 807 | 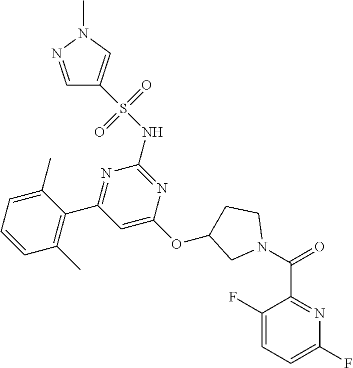 [SMILES: Cc1cccc(C)c1-c1cc(OC2CCN(C(=O)c3nc(F)ccc3F)C2)nc(NS(=O)(=O)c2cnn(C)c2)n1] | 1.35 | 569.166 | 570 | A |
| 808 | 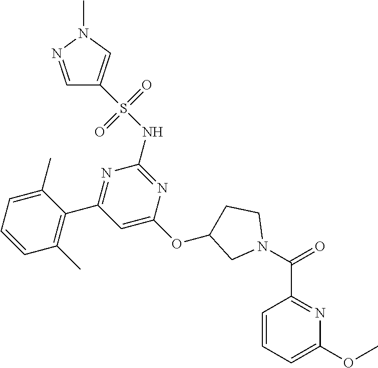 [SMILES: COc1cccc(C(=O)N2CCC(Oc3cc(-c4c(C)cccc4C)nc(NS(=O)(=O)c4cnn(C)c4)n3)C2)n1] | 1.48 | 563.195 | 564.21 | A |
| 809 | 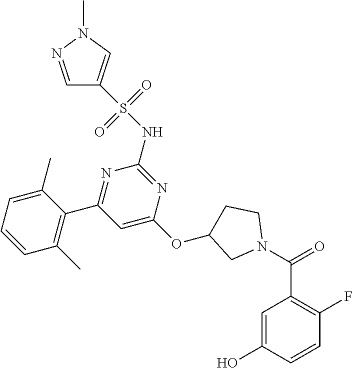 [SMILES: Cc1cccc(C)c1-c1cc(OC2CCN(C(=O)c3cc(O)ccc3F)C2)nc(NS(=O)(=O)c2cnn(C)c2)n1] | 1.29 | 566.175 | 567 | A |
| 810 | 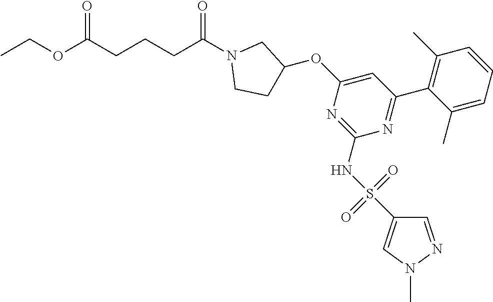 [SMILES: CCOC(=O)CCCC(=O)N1CCC(Oc2cc(-c3c(C)cccc3C)nc(NS(=O)(=O)c3cnn(C)c3)n2)C1] | 1.4 | 570.226 | 571.22 | A |
| 811 | 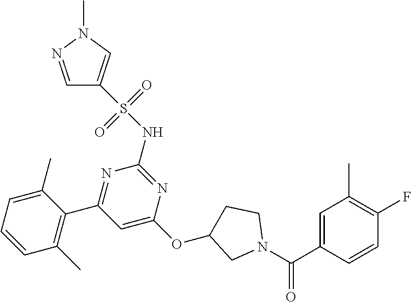 [SMILES: Cc1cc(C(=O)N2CCC(Oc3cc(-c4c(C)cccc4C)nc(NS(=O)(=O)c4cnn(C)c4)n3)C2)ccc1F] | 1.57 | 564.196 | 565.2 | A |
| 812 | 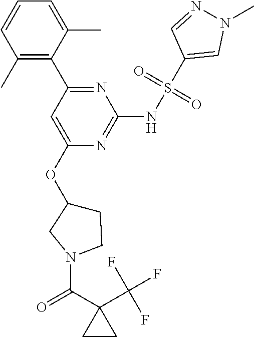 [SMILES: Cc1cccc(C)c1-c1cc(OC2CCN(C(=O)C3(C(F)(F)F)CC3)C2)nc(NS(=O)(=O)c2cnn(C)c2)n1] | 1.45 | 564.177 | 565.14 | A |
| 813 | 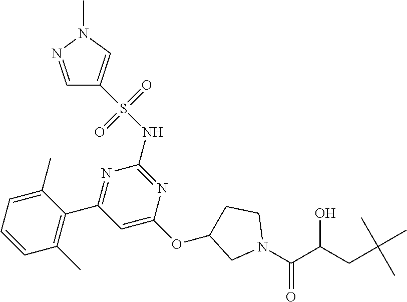 [SMILES: Cc1cccc(C)c1-c1cc(OC2CCN(C(=O)C(O)CC(C)(C)C)C2)nc(NS(=O)(=O)c2cnn(C)c2)n1] | 1.5 | 556.247 | 557.21 | A |
| 814 | 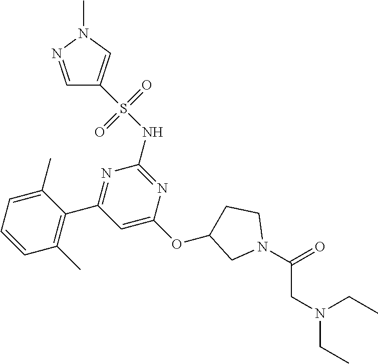 [SMILES: CCN(CC)CC(=O)N1CCC(Oc2cc(-c3c(C)cccc3C)nc(NS(=O)(=O)c3cnn(C)c3)n2)C1] | 1.07 | 541.247 | 542.27 | A |
| 815 | 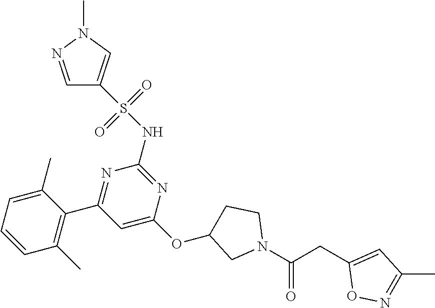 [SMILES: Cc1cc(CC(=O)N2CCC(Oc3cc(-c4c(C)cccc4C)nc(NS(=O)(=O)c4cnn(C)c4)n3)C2)on1] | 1.29 | 551.195 | 552.18 | A |
| 816 | 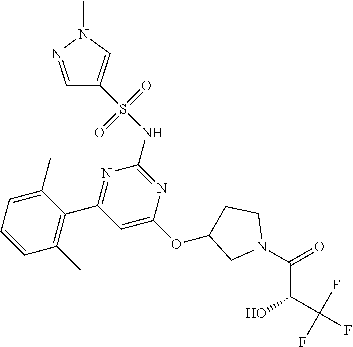 [SMILES: Cc1cccc(C)c1-c1cc(OC2CCN(C(=O)[C@@H](O)C(F)(F)F)C2)nc(NS(=O)(=O)c2cnn(C)c2)n1] | 1.31 | 554.156 | 555 | A |
| 817 | 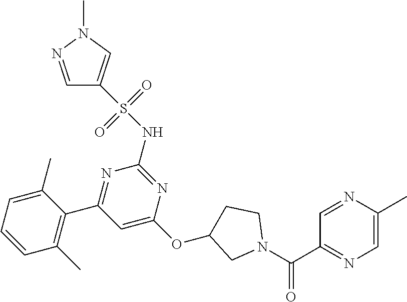 [SMILES: Cc1cnc(C(=O)N2CCC(Oc3cc(-c4c(C)cccc4C)nc(NS(=O)(=O)c4cnn(C)c4)n3)C2)cn1] | 1.29 | 548.195 | 549.14 | A |
| 818 | 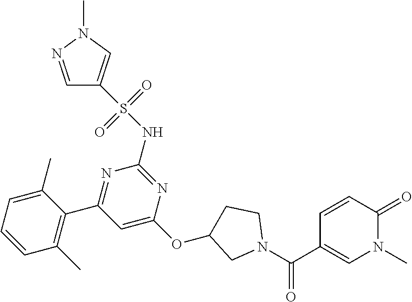 [SMILES: Cc1cccc(C)c1-c1cc(OC2CCN(C(=O)c3ccc(=O)n(C)c3)C2)nc(NS(=O)(=O)c2cnn(C)c2)n1] | 1.16 | 563.195 | 564.15 | A |
| 819 | 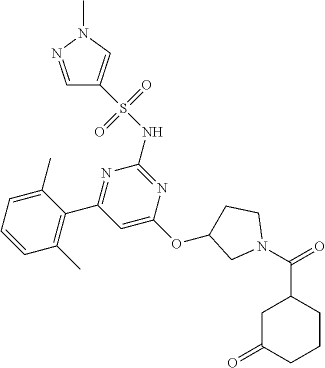 [SMILES: Cc1cccc(C)c1-c1cc(OC2CCN(C(=O)C3CCCC(=O)C3)C2)nc(NS(=O)(=O)c2cnn(C)c2)n1] | 1.19 | 552.215 | 553.18 | A |
| 820 | 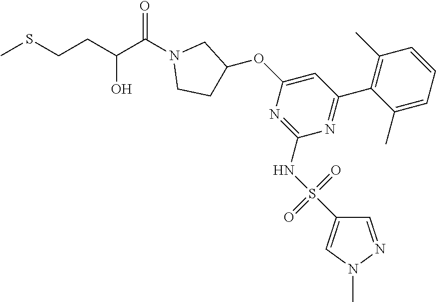 [SMILES: CSCCC(O)C(=O)N1CCC(Oc2cc(-c3c(C)cccc3C)nc(NS(=O)(=O)c3cnn(C)c3)n2)C1] | 1.29 | 560.188 | 561.17 | A |
| 821 | 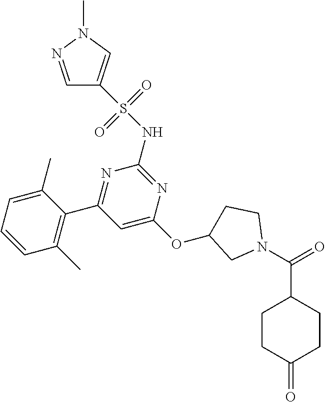 [SMILES: Cc1cccc(C)c1-c1cc(OC2CCN(C(=O)C3CCC(=O)CC3)C2)nc(NS(=O)(=O)c2cnn(C)c2)n1] | 1.24 | 552.215 | 553.18 | A |
| 822 | 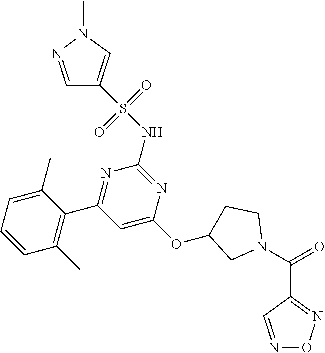 [SMILES: Cc1cccc(C)c1-c1cc(OC2CCN(C(=O)c3cnon3)C2)nc(NS(=O)(=O)c2cnn(C)c2)n1] | 1.28 | 524.159 | 525.15 | A |
| 823 | 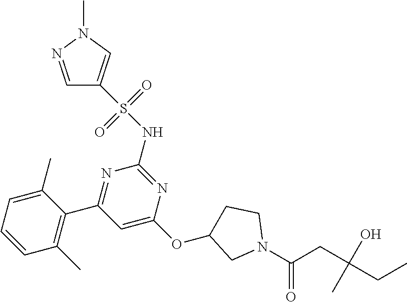 [SMILES: CCC(C)(O)CC(=O)N1CCC(Oc2cc(-c3c(C)cccc3C)nc(NS(=O)(=O)c3cnn(C)c3)n2)C1] | 1.33 | 542.231 | 543 | A |
| 824 | 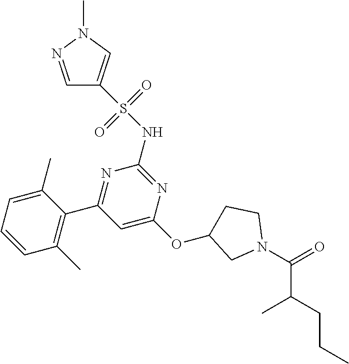 [SMILES: CCCC(C)C(=O)N1CCC(Oc2cc(-c3c(C)cccc3C)nc(NS(=O)(=O)c3cnn(C)c3)n2)C1] | 1.56 | 526.236 | 527.2 | A |
| 825 | 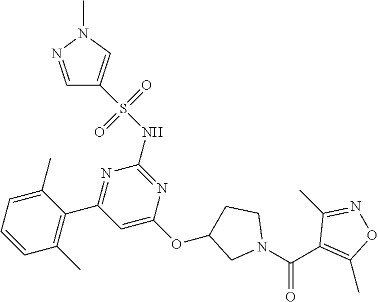 [SMILES: Cc1cccc(C)c1-c1cc(OC2CCN(C(=O)c3c(C)noc3C)C2)nc(NS(=O)(=O)c2cnn(C)c2)n1] | 1.31 | 551.195 | 552.18 | A |
| 826 | 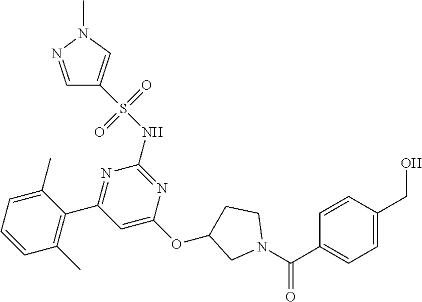 [SMILES: Cc1cccc(C)c1-c1cc(OC2CCN(C(=O)c3ccc(CO)cc3)C2)nc(NS(=O)(=O)c2cnn(C)c2)n1] | 1.24 | 562.2 | 563.16 | A |
| 827 | 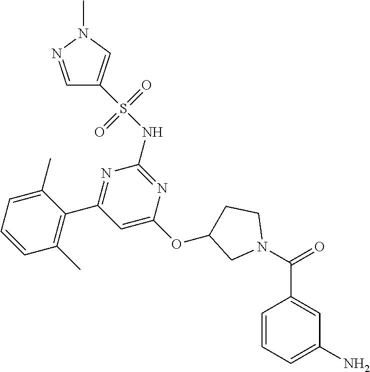 [SMILES: Cc1cccc(C)c1-c1cc(OC2CCN(C(=O)c3cccc(N)c3)C2)nc(NS(=O)(=O)c2cnn(C)c2)n1] | 1.04 | 547.2 | 548 | A |
| 828 | 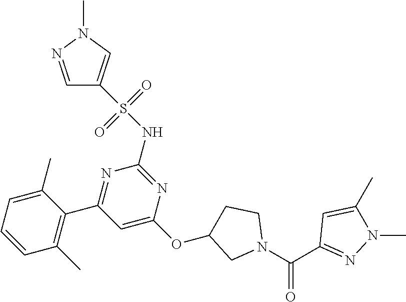 [SMILES: Cc1cccc(C)c1-c1cc(OC2CCN(C(=O)c3cc(C)n(C)n3)C2)nc(NS(=O)(=O)c2cnn(C)c2)n1] | 1.33 | 550.211 | 551.19 | A |
| 829 | 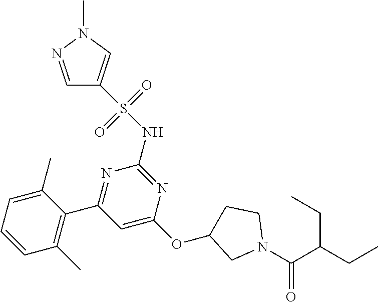 [SMILES: CCC(CC)C(=O)N1CCC(Oc2cc(-c3c(C)cccc3C)nc(NS(=O)(=O)c3cnn(C)c3)n2)C1] | 1.52 | 526.236 | 527 | A |
| 830 | 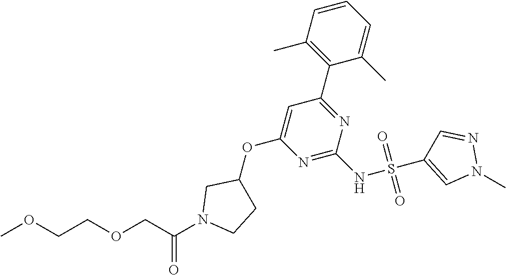 | 1.2 | 544.21 | 545.18 | A |
| 831 | 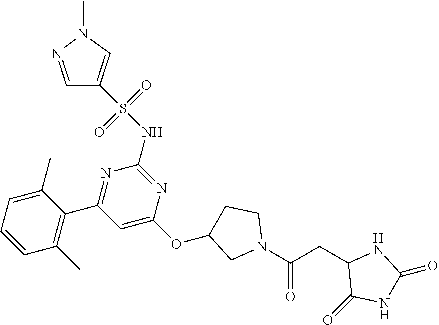 [SMILES: Cc1cccc(C)c1-c1cc(OC2CCN(C(=O)CC3NC(=O)NC3=O)C2)nc(NS(=O)(=O)c2cnn(C)c2)n1] | 1.1 | 568.185 | 569.17 | A |
| 832 | 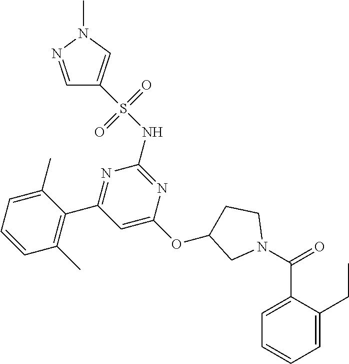 [SMILES: CCc1ccccc1C(=O)N1CCC(Oc2cc(-c3c(C)cccc3C)nc(NS(=O)(=O)c3cnn(C)c3)n2)C1] | 1.57 | 560.221 | 561 | A |
| 833 | 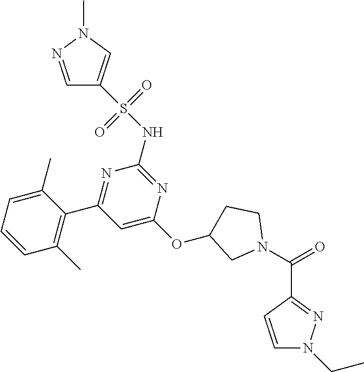 [SMILES: CCn1ccc(C(=O)N2CCC(Oc3cc(-c4c(C)cccc4C)nc(NS(=O)(=O)c4cnn(C)c4)n3)C2)n1] | 1.37 | 550.211 | 551.19 | A |
| 834 | 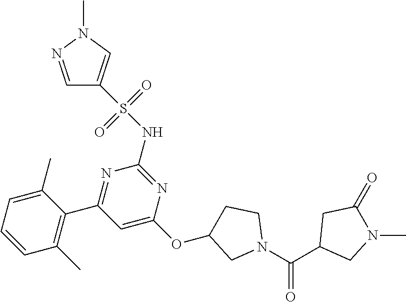 [SMILES: Cc1cccc(C)c1-c1cc(OC2CCN(C(=O)C3CC(=O)N(C)C3)C2)nc(NS(=O)(=O)c2cnn(C)c2)n1] | 1.11 | 553.211 | 554.17 | A |
| 835 | 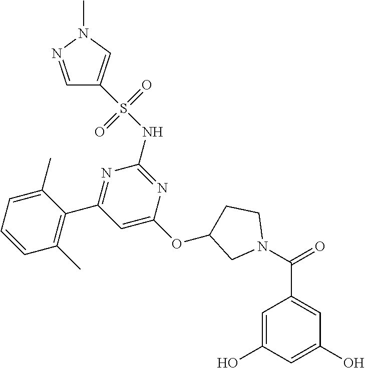 [SMILES: Cc1cccc(C)c1-c1cc(OC2CCN(C(=O)c3cc(O)cc(O)c3)C2)nc(NS(=O)(=O)c2cnn(C)c2)n1] | 1.2 | 564.179 | 565.14 | A |
| 836 | 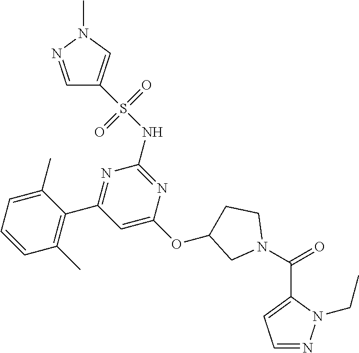 [SMILES: CCn1nccc1C(=O)N1CCC(Oc2cc(-c3c(C)cccc3C)nc(NS(=O)(=O)c3cnn(C)c3)n2)C1] | 1.34 | 550.211 | 551.19 | A |
| 837 | 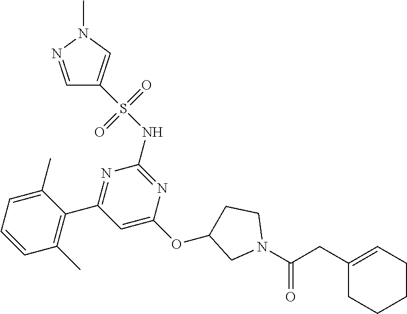 [SMILES: Cc1cccc(C)c1-c1cc(OC2CCN(C(=O)CC3=CCCCC3)C2)nc(NS(=O)(=O)c2cnn(C)c2)n1] | 1.59 | 550.236 | 551 | A |
| 838 | 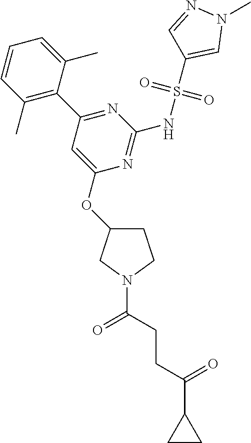 [SMILES: Cc1cccc(C)c1-c1cc(OC2CCN(C(=O)CCC(=O)C3CC3)C2)nc(NS(=O)(=O)c2cnn(C)c2)n1] | 1.31 | 552.215 | 553.18 | A |
| 839 | 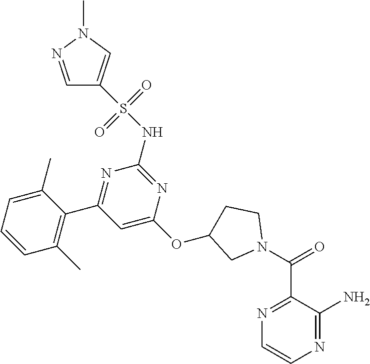 [SMILES: Cc1cccc(C)c1-c1cc(OC2CCN(C(=O)c3nccnc3N)C2)nc(NS(=O)(=O)c2cnn(C)c2)n1] | 1.22 | 549.191 | 550 | A |
| 840 | 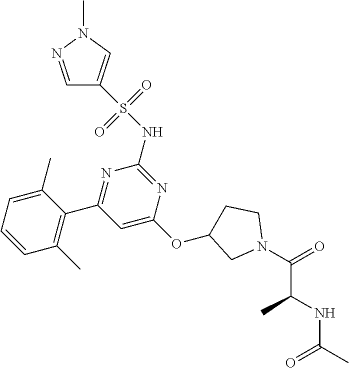 [SMILES: CC(=O)N[C@@H](C)C(=O)N1CCC(Oc2cc(-c3c(C)cccc3C)nc(NS(=O)(=O)c3cnn(C)c3)n2)C1] | 1.24 | 541.211 | 541.94 | A |
| 841 | 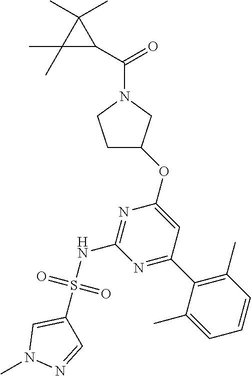 [SMILES: Cc1cccc(C)c1-c1cc(OC2CCN(C(=O)C3C(C)(C)C3(C)C)C2)nc(NS(=O)(=O)c2cnn(C)c2)n1] | 1.65 | 552.252 | 553.05 | A |
| 842 | 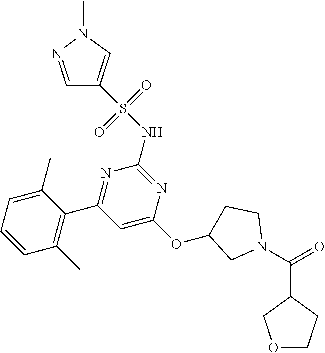 [SMILES: Cc1cccc(C)c1-c1cc(OC2CCN(C(=O)C3CCOC3)C2)nc(NS(=O)(=O)c2cnn(C)c2)n1] | 1.29 | 526.2 | 526.93 | A |
| 843 | 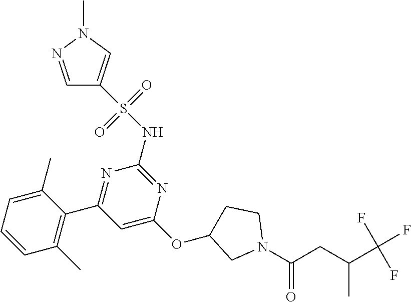 [SMILES: Cc1cccc(C)c1-c1cc(OC2CCN(C(=O)CC(C)C(F)(F)F)C2)nc(NS(=O)(=O)c2cnn(C)c2)n1] | 1.55 | 566.192 | 566.93 | A |
| 844 | 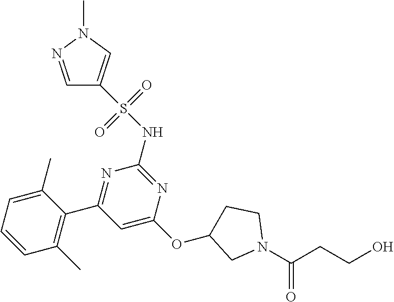 [SMILES: Cc1cccc(C)c1-c1cc(OC2CCN(C(=O)CCO)C2)nc(NS(=O)(=O)c2cnn(C)c2)n1] | 1.1 | 500.184 | 501.16 | A |
| 845 | 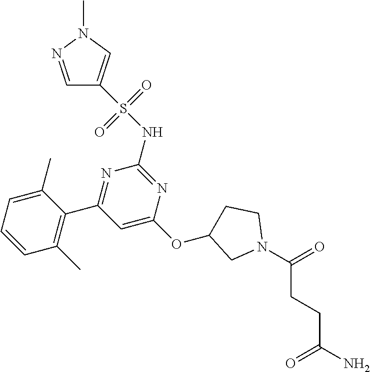 [SMILES: Cc1cccc(C)c1-c1cc(OC2CCN(C(=O)CCC(N)=O)C2)nc(NS(=O)(=O)c2cnn(C)c2)n1] | 1.08 | 527.195 | 528.19 | A |
| 846 | 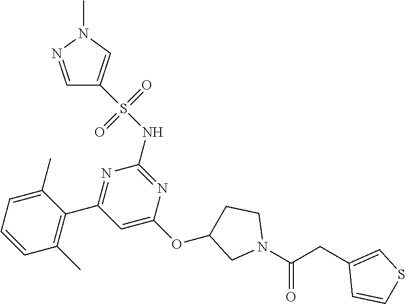 [SMILES: Cc1cccc(C)c1-c1cc(OC2CCN(C(=O)Cc3ccsc3)C2)nc(NS(=O)(=O)c2cnn(C)c2)n1] | 1.41 | 552.161 | 553 | A |
| 847 | 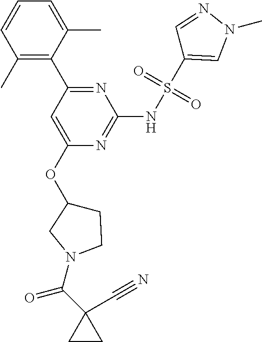 [SMILES: Cc1cccc(C)c1-c1cc(OC2CCN(C(=O)C3(C#N)CC3)C2)nc(NS(=O)(=O)c2cnn(C)c2)n1] | 1.39 | 521.185 | 521.96 | A |
| 848 | 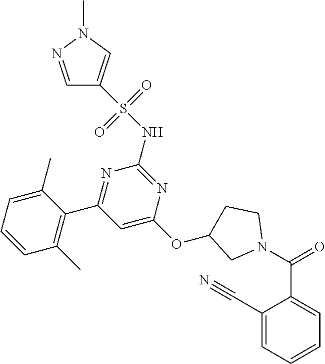 [SMILES: Cc1cccc(C)c1-c1cc(OC2CCN(C(=O)c3ccccc3C#N)C2)nc(NS(=O)(=O)c2cnn(C)c2)n1] | 1.42 | 557.185 | 557.89 | A |
| 849 | 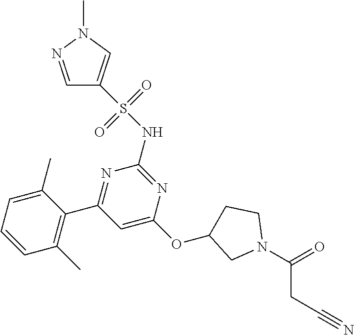 [SMILES: Cc1cccc(C)c1-c1cc(OC2CCN(C(=O)CC#N)C2)nc(NS(=O)(=O)c2cnn(C)c2)n1] | 1.27 | 495.169 | 495.83 | A |
| 850 | 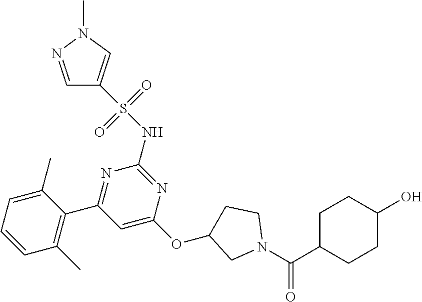 [SMILES: Cc1cccc(C)c1-c1cc(OC2CCN(C(=O)C3CCC(O)CC3)C2)nc(NS(=O)(=O)c2cnn(C)c2)n1] | 1.25 | 554.231 | 555.02 | A |
| 851 | 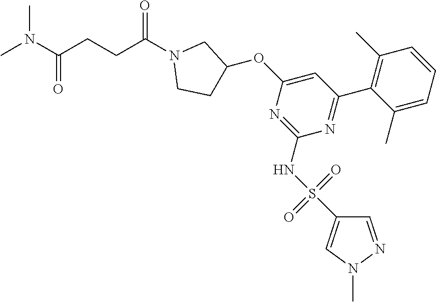 [SMILES: Cc1cccc(C)c1-c1cc(OC2CCN(C(=O)CCC(=O)N(C)C)C2)nc(NS(=O)(=O)c2cnn(C)c2)n1] | 1.13 | 555.226 | 556 | A |
| 852 | 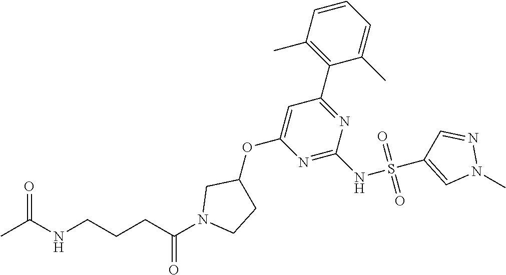 [SMILES: CC(=O)NCCCC(=O)N1CCC(Oc2cc(-c3c(C)cccc3C)nc(NS(=O)(=O)c3cnn(C)c3)n2)C1] | 1.21 | 555.226 | 555.99 | A |
| 853 | 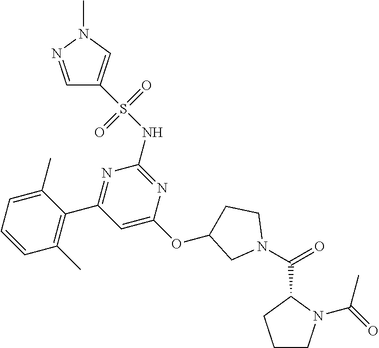 [SMILES: CC(=O)N1CCC[C@@H]1C(=O)N1CCC(Oc2cc(-c3c(C)cccc3C)nc(NS(=O)(=O)c3cnn(C)c3)n2)C1] | 1.28 | 567.226 | 567.95 | A |
| 854 | 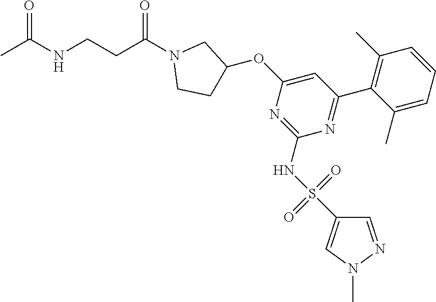 [SMILES: CC(=O)NCCC(=O)N1CCC(Oc2cc(-c3c(C)cccc3C)nc(NS(=O)(=O)c3cnn(C)c3)n2)C1] | 1.19 | 541.211 | 542.07 | A |
| 855 | 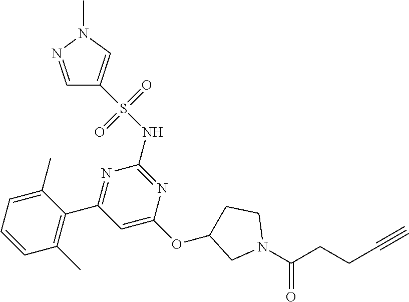 [SMILES: C#CCCC(=O)N1CCC(Oc2cc(-c3c(C)cccc3C)nc(NS(=O)(=O)c3cnn(C)c3)n2)C1] | 1.28 | 508.189 | 509 | A |
| 856 | 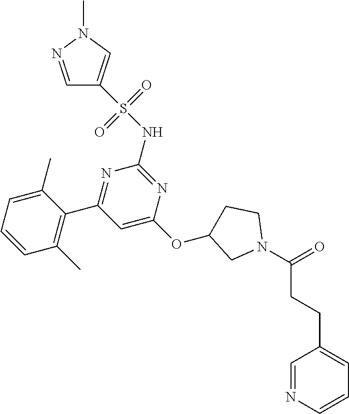 [SMILES: Cc1cccc(C)c1-c1cc(OC2CCN(C(=O)CCc3cccnc3)C2)nc(NS(=O)(=O)c2cnn(C)c2)n1] | 0.93 | 561.216 | 562 | A |
| 857 | 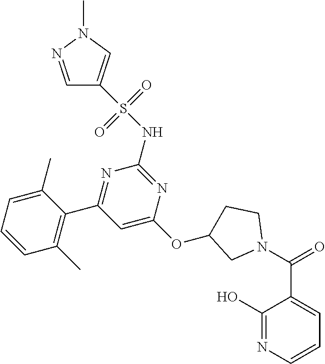 [SMILES: Cc1cccc(C)c1-c1cc(OC2CCN(C(=O)c3cccnc3O)C2)nc(NS(=O)(=O)c2cnn(C)c2)n1] | 1.18 | 549.179 | 549.9 | A |
| 858 | 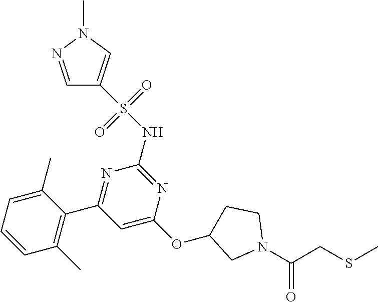 [SMILES: CSCC(=O)N1CCC(Oc2cc(-c3c(C)cccc3C)nc(NS(=O)(=O)c3cnn(C)c3)n2)C1] | 1.35 | 516.161 | 516.85 | A |
| 859 | 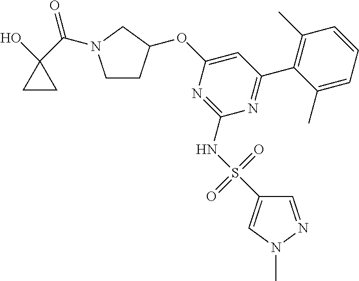 [SMILES: Cc1cccc(C)c1-c1cc(OC2CCN(C(=O)C3(O)CC3)C2)nc(NS(=O)(=O)c2cnn(C)c2)n1] | 1.13 | 512.184 | 513 | A |
| 860 | 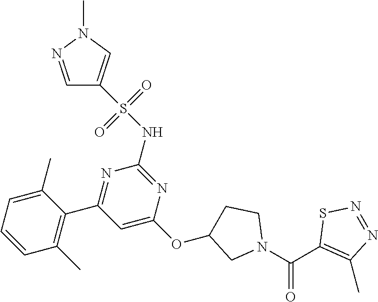 [SMILES: Cc1cccc(C)c1-c1cc(OC2CCN(C(=O)c3snnc3C)C2)nc(NS(=O)(=O)c2cnn(C)c2)n1] | 1.39 | 554.152 | 554.94 | A |
| 861 | 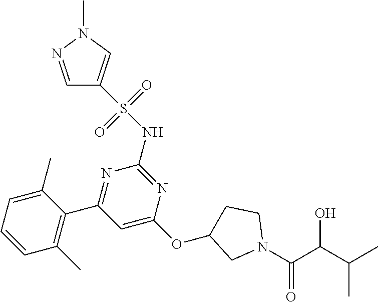 [SMILES: Cc1cccc(C)c1-c1cc(OC2CCN(C(=O)C(O)C(C)C)C2)nc(NS(=O)(=O)c2cnn(C)c2)n1] | 1.39 | 528.215 | 529 | A |
| 862 | 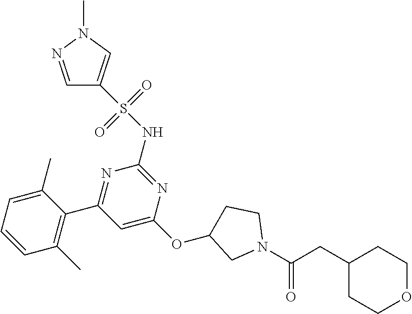 [SMILES: Cc1cccc(C)c1-c1cc(OC2CCN(C(=O)CC3CCOCC3)C2)nc(NS(=O)(=O)c2cnn(C)c2)n1] | 1.35 | 554.231 | 555.01 | A |
| 863 | 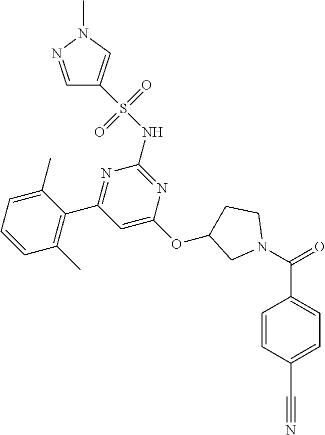 [SMILES: Cc1cccc(C)c1-c1cc(OC2CCN(C(=O)c3ccc(C#N)cc3)C2)nc(NS(=O)(=O)c2cnn(C)c2)n1] | 1.44 | 557.185 | 557.9 | A |
| 864 | 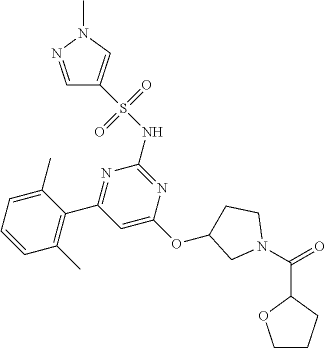 [SMILES: Cc1cccc(C)c1-c1cc(OC2CCN(C(=O)C3CCCO3)C2)nc(NS(=O)(=O)c2cnn(C)c2)n1] | 1.2 | 526.2 | 527 | A |
| 865 | 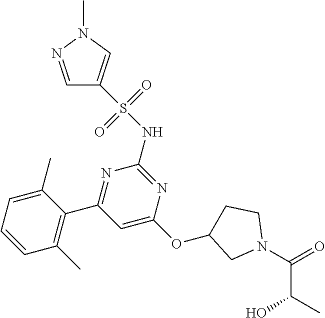 [SMILES: Cc1cccc(C)c1-c1cc(OC2CCN(C(=O)[C@H](C)O)C2)nc(NS(=O)(=O)c2cnn(C)c2)n1] | 1.22 | 500.184 | 500.95 | A |
| 866 | 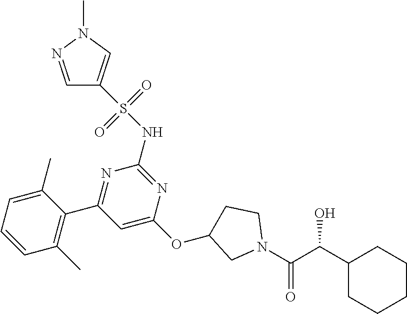 [SMILES: Cc1cccc(C)c1-c1cc(OC2CCN(C(=O)[C@H](O)C3CCCCC3)C2)nc(NS(=O)(=O)c2cnn(C)c2)n1] | 1.55 | 568.247 | 569.06 | A |
| 867 | 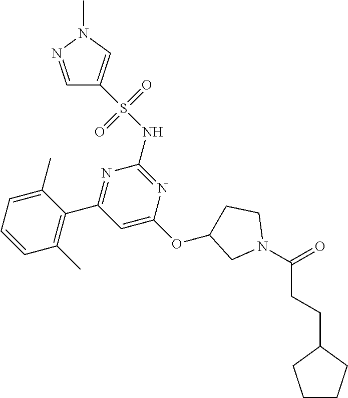 [SMILES: Cc1cccc(C)c1-c1cc(OC2CCN(C(=O)CCC3CCCC3)C2)nc(NS(=O)(=O)c2cnn(C)c2)n1] | 1.66 | 552.252 | 553.06 | A |
| 868 | 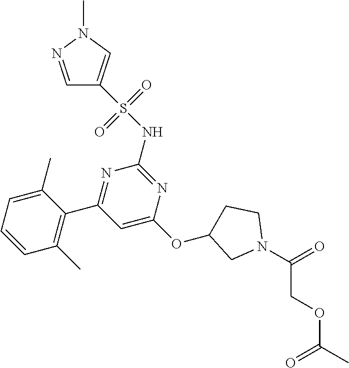 [SMILES: CC(=O)OCC(=O)N1CCC(Oc2cc(-c3c(C)cccc3C)nc(NS(=O)(=O)c3cnn(C)c3)n2)C1] | 1.28 | 528.179 | 528.95 | A |
| 869 | 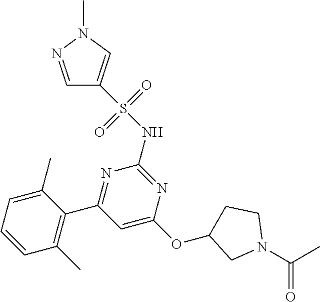 [SMILES: CC(=O)N1CCC(Oc2cc(-c3c(C)cccc3C)nc(NS(=O)(=O)c3cnn(C)c3)n2)C1] | 1.27 | 470.174 | 470.79 | A |
| 870 | 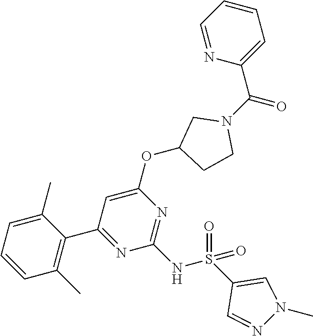 [SMILES: Cc1cccc(C)c1-c1cc(OC2CCN(C(=O)c3ccccn3)C2)nc(NS(=O)(=O)c2cnn(C)c2)n1] | 1.24 | 533.185 | 534 | A |
| 871 | 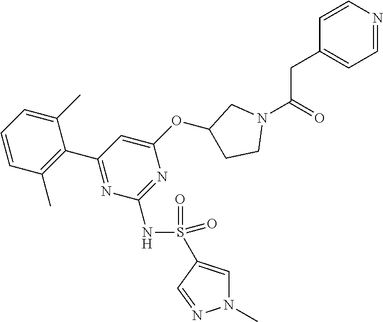 [SMILES: Cc1cccc(C)c1-c1cc(OC2CCN(C(=O)Cc3ccncc3)C2)nc(NS(=O)(=O)c2cnn(C)c2)n1] | 0.91 | 547.2 | 548 | Q |
| 872 | 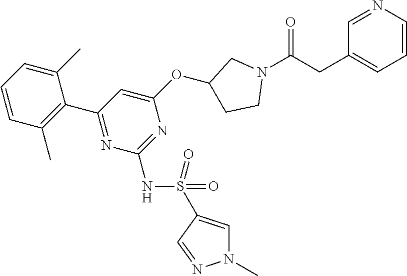 [SMILES: Cc1cccc(C)c1-c1cc(OC2CCN(C(=O)Cc3cccnc3)C2)nc(NS(=O)(=O)c2cnn(C)c2)n1] | 0.9 | 547.2 | 548 | A |
| 873 | 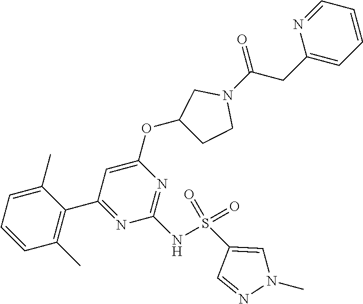 [SMILES: Cc1cccc(C)c1-c1cc(OC2CCN(C(=O)Cc3ccccn3)C2)nc(NS(=O)(=O)c2cnn(C)c2)n1] | 0.91 | 547.2 | 548 | A |
| 874 | 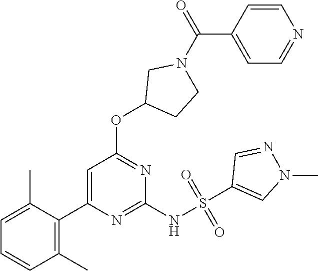 [SMILES: Cc1cccc(C)c1-c1cc(OC2CCN(C(=O)c3ccncc3)C2)nc(NS(=O)(=O)c2cnn(C)c2)n1] | 0.96 | 533.185 | 534 | A |
| 875 | 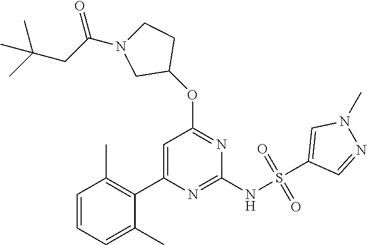 [SMILES: Cc1cccc(C)c1-c1cc(OC2CCN(C(=O)CC(C)(C)C)C2)nc(NS(=O)(=O)c2cnn(C)c2)n1] | 1.55 | 526.236 | 527 | A |
| 876 | 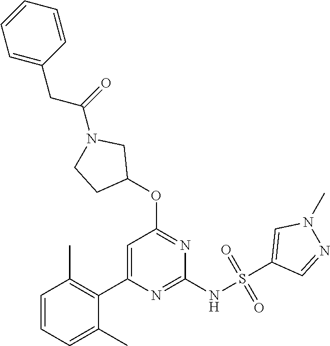 [SMILES: Cc1cccc(C)c1-c1cc(OC2CCN(C(=O)Cc3ccccc3)C2)nc(NS(=O)(=O)c2cnn(C)c2)n1] | 1.45 | 546.205 | 547 | A |
| 877 | 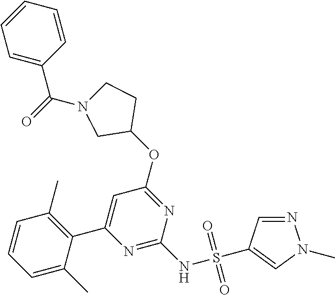 [SMILES: Cc1cccc(C)c1-c1cc(OC2CCN(C(=O)c3ccccc3)C2)nc(NS(=O)(=O)c2cnn(C)c2)n1] | 1.42 | 532.189 | 533 | A |
| 878 | 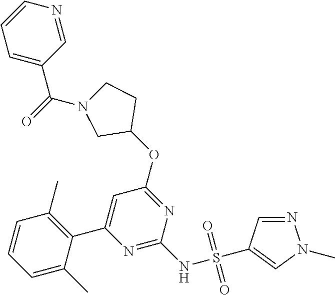 [SMILES: Cc1cccc(C)c1-c1cc(OC2CCN(C(=O)c3cccnc3)C2)nc(NS(=O)(=O)c2cnn(C)c2)n1] | 1 | 533.185 | 534 | A |
| 879 | 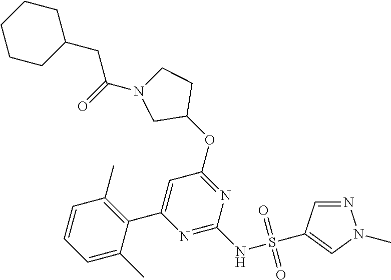 [SMILES: Cc1cccc(C)c1-c1cc(OC2CCN(C(=O)CC3CCCCC3)C2)nc(NS(=O)(=O)c2cnn(C)c2)n1] | 1.69 | 552.252 | 553 | A |

| Compound | |
|---|---|
| number | NMR |
| 736 | ¹H NMR (400 MHz, DMSO-d₆) δ 8.29 (d, J = 4.9 Hz, 1H), 7.76 (d, J = 6.8 Hz, |
| | 1H), 7.24 (t, J = 8.3, Hz, 1H), 7.12 (d, J = 10.4 Hz, 2H), 6.42 (d, J = 9.4 Hz, 1H), |
| | 5.44 (s, 1H), 3.99-3.78 (m, 6H), 3.73-3.58 (m, 3H), 3.50-3.36 (m, 2H), 2.08 |
| | (s, 3H), 2.02 (s, 6H), 1.77-1.38 (m, 6H), 1.04 (dd, J = 48.7, 37.3 Hz, 6H), 0.70 |
| | (d, J = 65.4 Hz, 3H). |
| 739 | ¹H NMR (400 MHz, DMSO-d₆) δ 8.30 (d, J = 10.7 Hz, 1H), 7.79 (d, J = 8.2 Hz, |
| | 1H), 7.53 (d, J = 29.2 Hz, 1H), 7.23 (d, J = 7.7 Hz, 2H), 7.12 (d, J = 7.6 Hz, 2H), |
| | 6.42 (s, 1H), 5.54 (d, J = 13.2 Hz, 1H), 4.27 (d, J = 18.3 Hz, 1H), 3.95 (s, 1H), |
| | 3.88 (s, 4H), 3.72 (s, 2H), 2.33 (d, J = 52.2 Hz, 1H), 2.05 (d, J = 24.0 Hz, 8H). |
| 740 | ¹H NMR (400 MHz, DMSO-d₆) δ 8.31 (d, J = 15.6 Hz, 1H), 7.78 (s, 1H), 7.75 |
| | (dd, J = 7.7, 3.7 Hz, 1H), 7.63 (d, J = 8.3 Hz, 1H), 7.47 (ddd, J = 9.6, 5.8, 2.0 Hz, |
| | 1H), 7.35 (t, J = 7.4 Hz, 1H), 7.24 (q, J = 7.0 Hz, 1H), 7.12 (t, J = 7.7 Hz, 2H), |
| | 6.44 (d, J = 14.5 Hz, 1H), 5.57 (dd, J = 12.5, 8.1 Hz, 1H), 4.10-3.92 (m, 2H), |
| | 3.87 (d, J = 20.2 Hz, 3H), 3.79-3.60 (m, 2H), 2.48 (d, J = 3.9 Hz, 3H), 2.37- |
| | 2.16 (m, 2H), 2.10-1.96 (m, 7H). |
| 742 | ¹H NMR (400 MHz, DMSO-d₆) δ 11.57 (s, 1H), 8.26 (d, J = 15.2 Hz, 1H), 7.73 |
| | (d, J = 7.9 Hz, 1H), 7.24 (t, J = 7.5 Hz, 1H), 7.09 (dd, J = 25.5, 7.9 Hz, 4H), 6.91 |
| | (d, J = 8.3 Hz, 1H), 6.77 (d, J = 8.3 Hz, 1H), 6.29 (d, J = 48.5 Hz, 1H), 5.27 (d, |
| | J = 52.1 Hz, 1H), 3.85 (s, 3H), 3.67 (d, J = 46.1 Hz, 4H), 3.16-2.79 (m, 2H), |
| | 2.05 (d, J = 28.8 Hz, 9H), 1.50-1.29 (m, 6H). |
| 745 | ¹H NMR (400 MHz, DMSO-d₆) δ 8.26 (d, J = 15.0 Hz, 1H), 7.73 (d, J = 6.3 Hz, |
| | 1H), 7.24 (t, J = 7.5 Hz, 1H), 7.12 (d, J = 8.0 Hz, 3H), 7.06 (d, J = 8.6 Hz, 1H), |
| | 6.89 (d, J = 8.7 Hz, 1H), 6.76 (d, J = 8.6 Hz, 1H), 6.26 (d, J = 54.2 Hz, 1H), 5.27 |
| | (d, J = 55.1 Hz, 1H), 3.85 (d, J = 5.5 Hz, 3H), 3.71 (s, 3H), 3.52 (s, 3H), 3.21 (d, |
| | J = 25.6 Hz, 2H), 3.13-2.89 (m, 1H), 2.32-2.18 (m, 2H), 2.09-1.86 (m, 12H), |
| | 1.77-1.55 (m, 4H). |
| 746 | ¹H NMR (400 MHz, DMSO-d₆) δ 8.25 (d, J = 19.8 Hz, 1H), 7.72 (d, J = 11.9 Hz, |
| | 1H), 7.34 (t, J = 7.6 Hz, 1H), 7.21 (td, J = 15.2, 7.8 Hz, 7H), 6.94 (t, J = 6.4 Hz, |
| | 1H), 6.25 (d, J = 58.2 Hz, 1H), 5.25 (d, J = 74.1 Hz, 1H), 3.85 (s, 3H), 3.76 (s, |
| | 1H), 3.53 (s, 2H), 3.21 (s, 1H), 2.99 (d, J = 59.7 Hz, 1H), 2.42-2.13 (m, 3H), |
| | 2.05 (d, J = 28.2 Hz, 11H), 1.84-1.52 (m, 5H). |
| 747 | ¹H NMR (400 MHz, DMSO-d₆) δ 8.26 (d, J = 22.1 Hz, 1H), 7.80-7.67 (m, 1H), |
| | 7.23 (d, J = 7.5 Hz, 2H), 7.13 (d, J = 9.5 Hz, 2H), 7.10-6.89 (m, 3H), 6.80 (t, J = |
| | 8.1 Hz, 1H), 6.24 (d, J = 48.4 Hz, 1H), 5.27 (d, J = 66.5 Hz, 1H), 3.85 (s, 3H), |
| | 3.73 (dd, J = 13.8, 4.6 Hz, 1H), 3.54 (d, J = 14.1 Hz, 2H), 3.24 (s, 1H), 3.13- |
| | 2.86 (m, 1H), 2.39-2.19 (m, 2H), 2.08 (s, 3H), 1.99 (d, J = 16.4 Hz, 8H), 1.68 |
| | (d, J = 20.5 Hz, 3H), 1.55 (d, J = 13.0 Hz, 1H). |
| 760 | ¹H NMR (400 MHz, DMSO-d₆) δ 8.29 (d, J = 8.2 Hz, 1H), 7.77 (d, J = 2.5 Hz, |
| | 1H), 7.26 (dt, J = 16.0, 7.9 Hz, 3H), 7.12 (d, J = 10.5 Hz, 4H), 6.40 (d, J = 8.6 |
| | Hz, 1H), 5.50 (d, J = 31.0 Hz, 1H), 3.94-3.82 (m, 6H), 3.75 (d, J = 10.4 Hz, 1H), |
| | 3.63 (d, J = 7.1 Hz, 2H), 3.52-3.35 (m, 1H), 2.26 (s, 3H), 2.24-2.12 (m, 1H), |
| | 2.08 (s, 1H), 2.02 (s, 6H). |
| 761 | ¹H NMR (400 MHz, DMSO-d₆) δ 8.27 (d, J = 42.0 Hz, 1H), 7.73 (d, J = 33.1 Hz, |
| | 1H), 7.38 (s, 1H), 7.24 (d, J = 11.7 Hz, 2H), 7.14 (d, J = 6.1 Hz, 2H), 7.07 (d, J = |
| | 12.5 Hz, 1H), 6.98 (d, J = 7.1 Hz, 1H), 6.35 (d, J = 15.6 Hz, 1H), 5.48 (d, J = |
| | 20.8 Hz, 1H), 3.96 (s, 2H), 3.88 (s, 2H), 3.76 (s, 2H), 3.28 (t, J = 10.4 Hz, 1H), |
| | 2.23 (s, 1H), 2.02 (d, J = 12.6 Hz, 7H), 1.74-1.56 (m, 2H), 0.96-0.84 (m, 3H). |
| 762 | ¹H NMR (400 MHz, DMSO-d₆) δ 11.64 (s, 1H), 8.28 (s, 1H), 7.76 (s, 1H), 7.15 |
| | (d, J = 35.0 Hz, 7H), 6.44 (s, 1H), 5.48 (d, J = 19.4 Hz, 1H), 3.85 (d, J = 20.6 Hz, |
| | 4H), 3.58 (d, J = 42.9 Hz, 5H), 3.10-2.99 (m, 2H), 2.81-2.71 (m, 1H), 2.59 (d, |
| | J = 10.6 Hz, 2H), 2.45-2.38 (m, 1H), 2.17 (s, 2H), 2.02 (s, 6H). |
| 771 | ¹H NMR (400 MHz, DMSO-d₆) δ 11.66 (s, 1H), 8.32 (s, 1H), 7.77 (s, 1H), 7.24 |
| | (s, 1H), 7.14 (s, 2H), 7.02 (d, J = 7.4 Hz, 2H), 6.94 (d, J = 8.3 Hz, 1H), 6.47 (s, |
| | 1H), 5.51 (d, J = 25.1 Hz, 1H), 4.54-4.31 (m, 2H), 3.84 (d, J = 29.3 Hz, 4H), |
| | 3.77-3.52 (m, 4H), 2.22 (d, J = 10.4 Hz, 8H), 2.01 (s, 7H). |
| 776 | ¹H NMR (400 MHz, DMSO-d₆) δ 8.28 (s, 1H), 7.77 (d, J = 15.8 Hz, 1H), 7.57 (t, |
| | J = 7.6 Hz, 1H), 7.42-7.34 (m, 1H), 7.24 (s, 2H), 7.13 (d, J = 7.4 Hz, 3H), 7.03- |
| | 6.95 (m, 1H), 6.39 (s, 1H), 5.48 (d, J = 23.5 Hz, 1H), 3.85 (s, 5H), 3.76-3.58 |
| | (m, 6H), 2.18 (s, 1H), 2.01 (s, 6H). |
| 780 | ¹H NMR (400 MHz, DMSO-d₆) δ 11.65 (s, 1H), 8.31 (s, 1H), 7.78 (s, 1H), 7.24 |
| | (s, 1H), 7.14 (s, 2H), 6.92 (dd, J = 14.8, 7.5 Hz, 2H), 6.74 (d, J = 8.3 Hz, 1H), |
| | 6.45 (s, 1H), 5.51 (d, J = 37.1 Hz, 1H), 4.75 (s, 1H), 4.73-4.61 (m, 1H), 3.85 (d, |
| | J = 10.3 Hz, 4H), 3.78-3.49 (m, 3H), 2.35-2.14 (m, 7H), 2.05 (d, J = 38.2 Hz, |
| | 8H). |
| 782 | ¹H NMR (400 MHz, DMSO-d₆) δ 10.79 (s, 1H), 8.27 (d, J = 21.6 Hz, 1H), 7.75 |
| | (s, 1H), 7.51 (t, J = 8.7 Hz, 1H), 7.32 (d, J = 8.1 Hz, 1H), 7.23 (d, J = 6.9 Hz, |
| | 1H), 7.13 (d, J = 7.6 Hz, 3H), 7.05 (s, 1H), 6.97 (s, 1H), 6.40 (s, 1H), 5.47 (d, J = |
| | 22.3 Hz, 1H), 3.83 (d, J = 32.5 Hz, 4H), 3.66-3.61 (m, 2H), 3.39-3.28 (m, 1H), |
| | 2.93 (d, J = 6.9 Hz, 2H), 2.70-2.57 (m, 2H), 2.21 (s, 2H), 2.01 (s, 6H). |
| 783 | ¹H NMR (400 MHz, DMSO-d₆) δ 8.30 (s, 1H), 7.78 (d, J = 13.3 Hz, 1H), 7.35 (d, |
| | J = 13.8 Hz, 2H), 7.24 (s, 2H), 7.13 (d, J = 10.5 Hz, 2H), 6.44 (d, J = 12.6 Hz, |
| | 1H), 5.54 (d, J = 38.3 Hz, 1H), 4.06 (d, J = 16.6 Hz, 1H), 3.86 (s, 7H), 3.63-3.34 |
| | (m, 3H), 2.31 (d, J = 49.1 Hz, 2H), 2.04 (d, J = 5.8 Hz, 6H). |
| 788 | ¹H NMR (400 MHz, DMSO-d₆) δ 8.22 (s, 1H), 8.16-8.06 (m, 1H), 7.65 (d, J = |
| | 5.6 Hz, 1H), 7.55-7.37 (m, 2H), 7.37-7.25 (m, 2H), 7.20 (d, J = 7.1 Hz, 1H), |
| | 7.10 (d, J = 7.2 Hz, 2H), 6.25-6.08 (m, 1H), 5.48 (dd, J = 37.2, 7.6 Hz, 2H), 3.91- |
| | 3.76 (m, 4H), 3.68 (d, J = 1.9 Hz, 2H), 2.20 (d, J = 33.7 Hz, 2H), 2.01 (d, J = |
| | 7.8 Hz, 6H). |
| 790 | ¹H NMR (400 MHz, DMSO-d₆) δ 8.24 (s, 1H), 7.74 (s, 1H), 7.23 (s, 1H), 7.13 (d, |
| | J = 2.7 Hz, 5H), 6.39-6.20 (m, 1H), 6.07 (s, 1H), 5.56-5.22 (m, 2H), 3.84 (s, |
| | 4H), 3.68 (d, J = 26.2 Hz, 2H), 2.20 (d, J = 29.7 Hz, 2H), 2.01 (d, J = 7.3 Hz, |
| | 6H). |
| 793 | ¹H NMR (400 MHz, DMSO-d₆) δ 11.65 (s, 1H), 8.30 (s, 1H), 7.74 (d, J = 25.1 |
| | Hz, 1H), 7.40-7.05 (m, 3H), 6.76 (s, 1H), 6.44 (s, 1H), 5.48 (d, J = 23.3 Hz, 1H), |
| | 3.86 (d, J = 4.2 Hz, 3H), 3.57 (d, J = 58.8 Hz, 3H), 2.84 (d, J = 48.3 Hz, 2H), |
| | 2.35-1.91 (m, 10H), 1.37 (s, 14H). |
| 797 | ¹H NMR (400 MHz, DMSO-d₆) δ 8.30 (s, 1H), 7.77 (d, J = 9.0 Hz, 1H), 7.37 (d, |
| | J = 10.7 Hz, 2H), 7.24 (d, J = 9.1 Hz, 2H), 7.14 (d, J = 7.6 Hz, 2H), 6.42 (d, J = |
| | 10.4 Hz, 1H), 5.53 (d, J = 35.5 Hz, 1H), 4.00 (d, J = 10.8 Hz, 1H), 3.86 (s, 3H), |
| | 3.82-3.67 (m, 4H), 2.28 (d, J = 48.4 Hz, 2H), 2.11-2.01 (m, 7H). |
| 798 | ¹H NMR (400 MHz, DMSO-d₆) δ 8.29 (s, 1H), 7.77 (d, J = 10.2 Hz, 1H), 7.23 (d, |
| | J = 8.2 Hz, 1H), 7.12 (d, J = 7.6 Hz, 2H), 6.79 (d, J = 7.1 Hz, 1H), 6.71-6.66 |
| | (m, 1H), 6.61 (t, J = 7.2 Hz, 1H), 6.39 (d, J = 4.6 Hz, 1H), 5.48 (d, J = 21.9 Hz, |
| | 1H), 3.95-3.81 (m, 3H), 3.72 (d, J = 6.9 Hz, 4H), 3.65 (dd, J = 13.7, 4.6 Hz, 1H), |
| | 3.53 (dd, J = 23.9, 9.8 Hz, 3H), 3.35 (d, J = 10.0 Hz, 1H), 2.29 (s, 1H), 2.08 (s, |
| | 2H), 2.02 (s, 6H). |
| 807 | ¹H NMR (400 MHz, DMSO-d₆) δ 8.27 (d, J = 32.4 Hz, 1H), 8.12 (d, J = 23.0 Hz, |
| | 1H), 7.74 (d, J = 30.8 Hz, 1H), 7.49-7.40 (m, 1H), 7.24 (d, J = 12.1 Hz, 1H), |
| | 7.13 (d, J = 11.4 Hz, 2H), 6.44 (d, J = 15.7 Hz, 1H), 5.50 (d, J = 44.9 Hz, 1H), |
| | 3.85 (d, J = 23.0 Hz, 6H), 3.78-3.58 (m, 2H), 3.53 (s, 2H), 2.34 (d, J = 13.9 Hz, |
| | 1H), 2.23-2.08 (m, 1H), 2.02 (d, J = 9.6 Hz, 6H). |
| 808 | ¹H NMR (400 MHz, DMSO-d₆) δ 9.70 (s, 1H), 8.27 (d, J = 30.5 Hz, 1H), 7.74 (d, |
| | J = 22.2 Hz, 1H), 7.26 (s, 1H), 7.16-7.02 (m, 3H), 6.83 (d, J = 12.0 Hz, 1H), |
| | 6.70 (s, 1H), 6.42 (d, J = 15.5 Hz, 1H), 5.49 (d, J = 31.3 Hz, 1H), 3.84 (d, J = |
| | 27.7 Hz, 3H), 3.77-3.57 (m, 2H), 2.24 (d, J = 52.8 Hz, 1H), 2.02 (d, J = 12.2 Hz, |
| | 7H). |
| 813 | ¹H NMR (400 MHz, DMSO-d₆) δ 8.28 (d, J = 18.9 Hz, 1H), 7.77 (s, 1H), 7.24 (s, |
| | 1H), 7.13 (d, J = 7.6 Hz, 2H), 6.41 (s, 1H), 5.57 (s, 1H), 4.24 (ddd, J = 34.6, 8.6, |
| | 3.2 Hz, 1H), 3.84 (d, J = 12.1 Hz, 5H), 2.22 (d, J = 30.3 Hz, 2H), 1.50 (d, J = |
| | 14.2 Hz, 1H), 1.42-1.29 (m, 1H), 0.94 (s, 9H). |
| 814 | ¹H NMR (400 MHz, DMSO-d₆) δ 9.40 (s, 1H), 8.30 (d, J = 11.2 Hz, 1H), 7.82- |
| | 7.74 (m, 1H), 7.25 (t, J = 8.0 Hz, 1H), 7.13 (d, J = 7.5 Hz, 2H), 6.40 (d, J = 12.5 |
| | Hz, 1H), 5.56 (d, J = 47.9 Hz, 1H), 4.23 (s, 4H), 3.87 (d, J = 3.9 Hz, 3H), 3.74 (s, |
| | 2H), 3.60 (s, 2H), 3.17 (s, 4H), 2.35-2.12 (m, 2H), 2.03 (s, 6H), 1.23 (q, J = 7.0 |
| | Hz, 6H). |
| 816 | ¹H NMR (400 MHz, DMSO-d₆) δ 8.29 (d, J = 7.5 Hz, 1H), 7.77 (s, 1H), 7.29- |
| | 7.21 (m, 1H), 7.13 (d, J = 7.6 Hz, 2H), 6.41 (s, 1H), 5.53 (d, J = 39.8 Hz, 1H), |
| | 4.91 (dd, J = 37.8, 7.2 Hz, 1H), 3.95 (d, J = 12.2 Hz, 1H), 3.86 (d, J = 7.4 Hz, |
| | 4H), 3.54-3.43 (m, 2H), 2.31-2.09 (m, 2H), 2.02 (s, 6H). |
| 817 | ¹H NMR (400 MHz, DMSO-d₆) δ 8.87 (s, 1H), 8.59 (d, J = 7.5 Hz, 1H), 8.29 (d, |
| | J = 22.6 Hz, 1H), 7.82-7.71 (m, 1H), 7.21 (s, 1H), 7.17-7.08 (m, 2H), 6.42 (d, |
| | J = 18.8 Hz, 1H), 5.55 (s, 1H), 4.15 (d, J = 13.2 Hz, 1H), 3.86 (d, J = 15.6 Hz, |
| | 9H), 2.57 (s, 3H), 2.08 (s, 3H), 2.02 (d, J = 14.1 Hz, 6H). |
| 819 | ¹H NMR (400 MHz, DMSO-d₆) δ 8.28 (d, J = 8.5 Hz, 1H), 7.76 (d, J = 5.0 Hz, |
| | 1H), 7.24 (t, J = 7.5 Hz, 1H), 7.13 (d, J = 7.6 Hz, 2H), 6.42 (s, 1H), 5.49 (d, J = |
| | 29.9 Hz, 1H), 3.86 (d, J = 4.5 Hz, 4H), 3.69 (d, J = 9.1 Hz, 2H), 3.39 (t, J = 8.5 |
| | Hz, 1H), 3.02 (d, J = 40.4 Hz, 1H), 2.34 (s, 2H), 2.20 (d, J = 21.6 Hz, 3H), 2.03 |
| | (s, 7H), 1.88 (s, 2H), 1.69 (s, 2H). |
| 824 | ¹H NMR (400 MHz, DMSO-d₆) δ 8.29 (s, 1H), 7.77 (d, J = 5.9 Hz, 1H), 7.24 (t, |
| | J = 7.5 Hz, 1H), 7.13 (d, J = 8.3 Hz, 2H), 6.41 (s, 1H), 5.47 (d, J = 19.9 Hz, 1H), |
| | 3.87 (d, J = 3.7 Hz, 4H), 3.73-3.56 (m, 2H), 2.25 (d, J = 59.1 Hz, 2H), 2.02 (s, |
| | 6H), 1.50 (s, 1H), 1.24 (d, J = 46.0 Hz, 3H), 1.03-0.91 (m, 3H), 0.90-0.80 (m, |
| | 2H), 0.71 (t, J = 7.1 Hz, 1H). |
| 829 | ¹H NMR (400 MHz, DMSO-d₆) δ 8.30 (s, 1H), 7.77 (d, J = 8.1 Hz, 1H), 7.29- |
| | 7.19 (m, 1H), 7.12 (d, J = 7.6 Hz, 2H), 6.37 (d, J = 10.1 Hz, 1H), 5.44 (s, 1H), |
| | 3.90-3.80 (m, 4H), 3.71 (d, J = 12.9 Hz, 1H), 3.66-3.39 (m, 3H), 2.35 (d, J = |
| | 55.1 Hz, 3H), 2.01 (s, 6H), 1.45 (d, J = 47.5 Hz, 4H), 0.84 (q, J = 7.3 Hz, 3H), |
| | 0.78 (d, J = 14.8 Hz, 1H), 0.66 (t, J = 7.4 Hz, 1H). |
| 837 | ¹H NMR (400 MHz, DMSO-d₆) δ 8.29 (d, J = 5.6 Hz, 1H), 7.76 (d, J = 2.2 Hz, |
| | 1H), 7.24 (t, J = 7.5 Hz, 1H), 7.13 (d, J = 7.6 Hz, 2H), 6.39 (d, J = 12.6 Hz, 1H), |
| | 5.41 (d, J = 25.0 Hz, 2H), 3.86 (d, J = 3.9 Hz, 3H), 3.85-3.78 (m, 1H), 3.72- |
| | 3.46 (m, 3H), 3.37 (d, J = 10.2 Hz, 1H), 2.91 (d, J = 28.3 Hz, 2H), 2.24 (d, J = |
| | 42.6 Hz, 1H), 2.08 (s, 1H), 2.02 (s, 6H), 1.90 (d, J = 21.2 Hz, 4H), 1.49 (t, J = |
| | 28.1 Hz, 4H). |
| 840 | ¹H NMR (400 MHz, DMSO-d₆) δ 8.29 (t, J = 9.0 Hz, 1H), 8.19 (s, 1H), 7.76 (d, |
| | J = 5.8 Hz, 1H), 7.27-7.20 (m, 1H), 7.12 (d, J = 7.6 Hz, 2H), 6.41 (d, J = 4.3 |
| | Hz, 1H), 5.49 (d, J = 28.2 Hz, 1H), 4.55 (d, J = 13.6 Hz, 1H), 4.42 (d, J = 6.9 Hz, |
| | 1H), 3.87 (s, 3H), 3.71-3.33 (m, 3H), 2.25 (d, J = 48.2 Hz, 2H), 2.02 (s, 6H), |
| | 1.86-1.74 (m, 3H), 1.26-1.07 (m, 3H). |
| 843 | ¹H NMR (400 MHz, DMSO-d₆) δ 8.28 (d, J = 9.1 Hz, 1H), 7.76 (d, J = 3.8 Hz, |
| | 1H), 7.28-7.20 (m, 1H), 7.12 (d, J = 7.4 Hz, 2H), 6.39 (s, 1H), 5.48 (d, J = 23.8 |
| | Hz, 1H), 3.86 (d, J = 4.5 Hz, 4H), 3.59 (d, J = 52.7 Hz, 7H), 2.64 (s, 3H), 2.42- |
| | 2.13 (m, 5H), 2.02 (s, 6H), 1.09 (t, J = 11.2 Hz, 3H). |
| 846 | ¹H NMR (400 MHz, DMSO-d₆) δ 8.29 (d, J = 4.2 Hz, 1H), 7.77 (d, J = 7.6 Hz, |
| | 1H), 7.49-7.41 (m, 1H), 7.25 (t, J = 9.7 Hz, 2H), 7.13 (d, J = 7.3 Hz, 2H), 7.01 |
| | (dd, J = 10.5, 4.9 Hz, 1H), 6.40 (s, 1H), 5.48 (d, J = 17.9 Hz, 1H), 3.94-3.83 (m, |
| | 5H), 3.76-3.48 (m, 9H), 2.26 (d, J = 57.0 Hz, 2H), 2.02 (s, 7H). |
| 850 | ¹H NMR (400 MHz, DMSO-d₆) δ 8.28 (d, J = 6.7 Hz, 1H), 7.75 (d, J = 4.9 Hz, |
| | 1H), 7.23 (t, J = 7.5 Hz, 1H), 7.12 (d, J = 7.6 Hz, 2H), 6.40 (d, J = 6.0 Hz, 1H), |
| | 5.46 (d, J = 13.8 Hz, 1H), 3.86 (d, J = 3.9 Hz, 4H), 3.80 (s, 1H), 3.73-3.30 (m, |
| | 4H), 2.46-2.06 (m, 3H), 2.02 (s, 6H), 1.82-1.57 (m, 4H), 1.51-1.21 (m, 4H). |
| 853 | ¹H NMR (400 MHz, DMSO-d₆) δ 8.29 (s, 1H), 7.75 (d, J = 4.7 Hz, 1H), 7.24 (t, |
| | J = 7.5 Hz, 1H), 7.12 (d, J = 7.6 Hz, 2H), 6.41 (d, J = 7.7 Hz, 1H), 5.48 (d, J = |
| | 30.5 Hz, 1H), 4.81-4.34 (m, 2H), 3.86 (d, J = 6.4 Hz, 4H), 3.71 (d, J = 31.8 Hz, |
| | 2H), 3.50 (s, 2H), 3.36 (d, J = 7.1 Hz, 1H), 2.36-2.12 (m, 2H), 2.03 (d, J = 5.6 |
| | Hz, 6H), 1.96 (s, 3H), 1.75 (d, J = 45.1 Hz, 2H). |
| 867 | ¹H NMR (400 MHz, DMSO-d₆) δ 8.28 (d, J = 7.1 Hz, 1H), 7.76 (d, J = 4.7 Hz, |
| | 1H), 7.23 (t, J = 7.5 Hz, 1H), 7.12 (d, J = 7.6 Hz, 2H), 6.40 (d, J = 9.4 Hz, 1H), |
| | 5.47 (d, J = 22.3 Hz, 1H), 3.86 (d, J = 3.7 Hz, 4H), 3.68-3.35 (m, 5H), 2.32- |
| | 2.14 (m, 3H), 2.05 (d, J = 24.3 Hz, 7H), 1.71 (s, 3H), 1.51 (d, J = 32.2 Hz, 6H), |
| | 1.07 (s, 2H). |
| 869 | ¹H NMR (400 MHz, DMSO-d₆) δ 8.28 (s, 1H), 7.76 (s, 1H), 7.27-7.21 (m, 1H), |
| | 7.12 (d, J = 7.6 Hz, 2H), 6.40 (s, 1H), 5.47 (d, J = 20.6 Hz, 1H), 3.86 (d, J = 5.1 |
| | Hz, 4H), 2.18 (d, J = 18.8 Hz, 3H), 1.98 (s, 2H), 1.94 (s, 1H). |
| 870 | ¹H NMR (400 MHz, DMSO-d₆) δ 8.62 (s, 1H), 8.29 (d, J = 21.3 Hz, 1H), 7.96 (d, |
| | J = 7.8 Hz, 1H), 7.84-7.70 (m, 2H), 7.53 (s, 1H), 7.23 (d, J = 5.3 Hz, 1H), 7.12 |
| | (t, J = 7.1 Hz, 2H), 6.42 (d, J = 19.3 Hz, 1H), 5.53 (s, 1H), 4.11 (dd, J = 13.1, 4.3 |
| | Hz, 1H), 3.88 (s, 2H), 3.83 (s, 4H), 2.34-2.55 (m, 1H), 2.13 (d, J = 14.8 Hz, 1H), |
| | 2.01 (d, J = 15.5 Hz, 6H). |
| 871 | ¹H NMR (400 MHz, DMSO-d₆) δ 8.86 (d, J = 6.1 Hz, 2H), 8.30 (s, 1H), 7.96 (s, |
| | 2H), 7.78 (d, J = 10.6 Hz, 1H), 7.26 (s, 1H), 7.13 (s, 2H), 6.42 (d, J = 12.6 Hz, |
| | 1H), 5.54 (d, J = 38.7 Hz, 1H), 4.15 (d, J = 6.0 Hz, 2H), 4.01 (s, 1H), 3.86 (s, |
| | 4H), 3.52 (d, J = 13.7 Hz, 1H), 3.40 (s, 1H), 2.34 (s, 1H), 2.22 (s, 1H), 2.03 (d, J = |
| | 5.9 Hz, 7H). |
| 872 | ¹H NMR (400 MHz, DMSO-d₆) δ 8.85 (d, J = 5.4 Hz, 2H), 8.50 (s, 1H), 8.31 (s, |
| | 1H), 8.12-8.02 (m, 1H), 7.78 (d, J = 13.0 Hz, 1H), 7.24 (s, 1H), 7.12 (d, J = 3.5 |
| | Hz, 2H), 6.42 (d, J = 13.6 Hz, 1H), 5.55 (d, J = 40.8 Hz, 1H), 4.04 (d, J = 7.8 Hz, |
| | 3H), 3.86 (d, J = 3.4 Hz, 3H), 3.68 (d, J = 17.3 Hz, 3H), 3.52 (d, J = 13.7 Hz, |
| | 1H), 3.40 (s, 1H), 2.34 (s, 1H), 2.24 (s, 1H), 2.03 (d, J = 7.0 Hz, 7H). |
| 873 | ¹H NMR (400 MHz, DMSO-d₆) δ 8.89 (s, 1H), 8.53 (s, 1H), 8.32 (s, 1H), 7.96 (s, |
| | 2H), 7.78 (d, J = 11.7 Hz, 1H), 7.19 (d, J = 43.1 Hz, 3H), 6.42 (d, J = 11.9 Hz, |
| | 1H), 5.56 (d, J = 38.9 Hz, 1H), 4.32 (d, J = 10.9 Hz, 4H), 3.85 (s, 4H), 2.27 (s, |
| | 2H), 2.03 (d, J = 6.1 Hz, 8H). |
| 874 | ¹H NMR (400 MHz, DMSO-d₆) δ 8.84 (d, J = 5.0 Hz, 2H), 8.26 (d, J = 37.1 Hz, |
| | 1H), 7.86-7.69 (m, 3H), 7.23 (d, J = 13.5 Hz, 1H), 7.12 (d, J = 14.7 Hz, 2H), |
| | 6.42 (d, J = 9.5 Hz, 1H), 5.49 (d, J = 40.1 Hz, 1H), 3.85 (d, J = 19.7 Hz, 6H), |
| | 3.67-3.45 (m, 4H), 2.30 (s, 3H), 2.01 (d, J = 15.0 Hz, 6H). |
| 875 | ¹H NMR (400 MHz, DMSO-d₆) δ 8.28 (d, J = 4.9 Hz, 1H), 7.76 (d, J = 5.8 Hz, |
| | 1H), 7.23 (t, J = 7.5 Hz, 1H), 7.12 (d, J = 7.6 Hz, 2H), 6.38 (d, J = 5.4 Hz, 1H), |
| | 5.44 (d, J = 5.7 Hz, 1H), 3.86 (s, 3H), 3.82 (dd, J = 12.2, 4.5 Hz, 1H), 3.52 (d, J = |
| | 12.9 Hz, 3H), 3.37 (s, 1H), 2.35-2.20 (m, 1H), 2.18 (d, J = 2.4 Hz, 1H), 2.13 |
| | (s, 1H), 2.07 (d, J = 14.4 Hz, 1H), 2.02 (s, 6H), 0.98 (d, J = 18.6 Hz, 9H). |
| 876 | ¹H NMR (400 MHz, DMSO-d₆) δ 8.21 (s, 1H), 7.69 (d, J = 9.1 Hz, 1H), 7.20 (dd, |
| | J = 22.9, 9.0 Hz, 7H), 7.05 (d, J = 7.3 Hz, 2H), 6.32 (s, 1H), 5.41 (d, J = 19.7 Hz, |
| | 1H), 3.78 (d, J = 3.5 Hz, 4H), 3.68-3.50 (m, 5H), 2.18 (d, J = 59.9 Hz, 2H), 1.95 |
| | (s, 7H). |
| 877 | ¹H NMR (400 MHz, DMSO-d₆) δ 8.27 (d, J = 35.1 Hz, 1H), 7.74 (d, J = 22.2 Hz, |
| | 1H), 7.59-7.41 (m, 6H), 7.23 (d, J = 7.5 Hz, 1H), 7.12 (t, J = 8.2 Hz, 2H), 6.41 |
| | (d, J = 16.7 Hz, 1H), 5.49 (d, J = 35.3 Hz, 1H), 3.87 (t, J = 22.2 Hz, 5H), 2.26 (s, |
| | 1H), 2.03 (t, J = 18.6 Hz, 8H). |
| 878 | ¹H NMR (400 MHz, DMSO-d₆) δ 8.94 (d, J = 10.9 Hz, 1H), 8.82 (d, J = 5.0 Hz, |
| | 1H), 8.39-8.19 (m, 2H), 7.75 (d, J = 28.5 Hz, 2H), 7.32-7.09 (m, 3H), 6.40 (d, |
| | J = 15.0 Hz, 1H), 5.50 (d, J = 34.8 Hz, 1H), 4.05 (d, J = 11.5 Hz, 1H), 3.98-3.92 |
| | (m, 2H), 3.87 (s, 3H), 3.82 (s, 3H), 2.38-2.23 (m, 2H), 2.14 (s, 2H), 2.01 (d, J = |
| | 16.5 Hz, 6H). |
| 879 | ¹H NMR (400 MHz, DMSO-d₆) δ 8.28 (d, J = 6.0 Hz, 1H), 7.76 (d, J = 4.6 Hz, |
| | 1H), 7.23 (t, J = 7.3 Hz, 1H), 7.12 (d, J = 7.5 Hz, 2H), 6.39 (d, J = 7.5 Hz, 1H), |
| | 5.45 (d, J = 12.9 Hz, 1H), 3.86 (s, 4H), 2.35-1.98 (m, 11H), 1.65 (d, J = 27.5 |
| | Hz, 7H), 1.18 (d, J = 23.6 Hz, 4H), 0.92 (s, 2H). |

### Example 263: Preparation of Compound 880

### Step 1: N-[4-[1-[2-(3-chlorophenyl)ethyl]pyrrolidin-3-yl]oxy-6-(2,6-dimethylphenyl)pyrimidin-2-yl]-1-methyl-pyrazole-4-sulfonamide

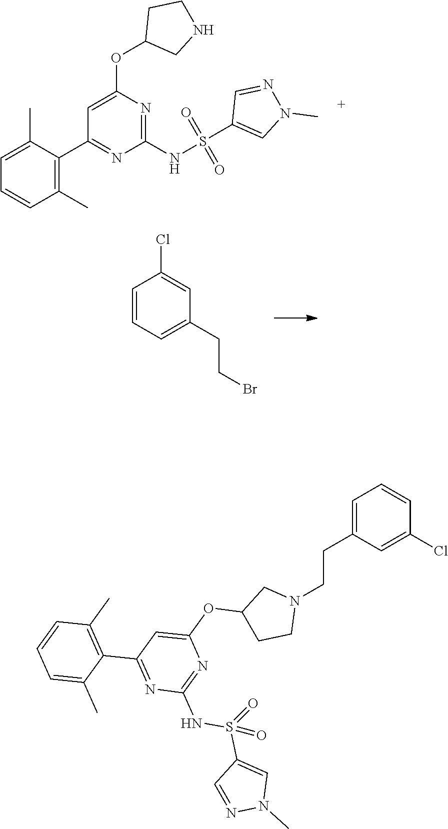

N-[4-(2,6-Dimethylphenyl)-6-pyrrolidin-3-yloxy-pyrimidin-2-yl]-1-methyl-pyrazole-4-sulfonamide (30 mg, 0.07001 mmol) in DMF (0.2 mL) was added to 1-(2-bromoethyl)-3-chloro-benzene (approximately 30.73 mg, 20.59 μL, 0.1400 mmol), followed by TEA (approximately 28.33 mg, 39.02 μL, 0.2800 mmol). The mixture was shaken at 60° C. for 1 hour. The reaction mixture was allowed to cool to room temperature, filtered and purified by reverse phase HPLC. N-[4-[1-[2-(3-chlorophenyl)ethyl]pyrrolidin-3-yl]oxy-6-(2,6-dimethylphenyl)pyrimidin-2-yl]-1-methyl-pyrazole-4-sulfonamide (6.7 mg). ESI-MS m/z calc. 566.1867, found 567.0 (M+1)⁺; Retention time: 1.28 minutes; LC method A. ¹H NMR (400 MHz, DMSO-d₆) δ 11.47-10.93 (m, 1H), 8.45-8.30 (m, 1H), 7.85-7.76 (m, 1H), 7.43 (q, J=2.0 Hz, 1H), 7.38-7.34 (m, 1H), 7.30-7.22 (m, 2H), 7.14 (dd, J=7.6, 2.9 Hz, 2H), 6.47 (s, 1H), 5.58-5.39 (m, 1H), 4.31-4.18 (m, 1H), 3.91-3.85 (m, 3H), 3.84-3.75 (m, 1H), 3.75-3.60 (m, 2H), 3.33-3.26 (m, 1H), 3.12-2.98 (m, 2H), 2.73-2.61 (m, 1H), 2.49-2.37 (m, 1H), 2.31-2.09 (m, 2H), 2.02 (s, 6H).

### Example 264: Preparation of Compound 881

### Step 1: N-[4-(2,6-Dimethylphenyl)-6-[1-(1-phenylethyl)pyrrolidin-3-yl]oxy-pyrimidin-2-yl]-1-methyl-pyrazole-4-sulfonamide

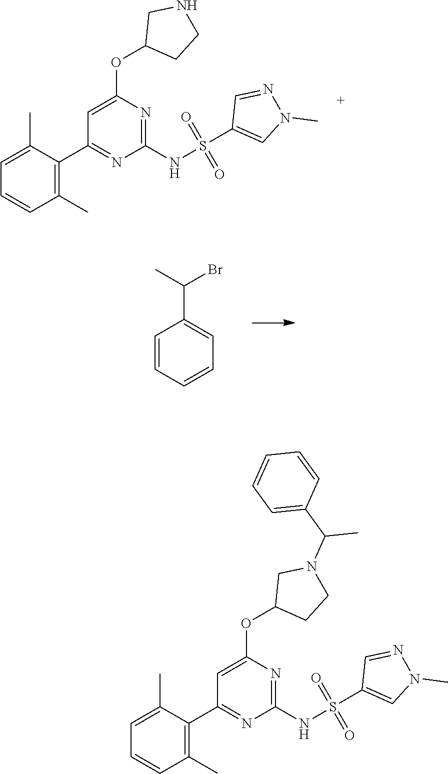

To a vial equipped with magnetic stir bar was added N-[4-(2,6-dimethylphenyl)-6-pyrrolidin-3-yloxy-pyrimidin-2-yl]-1-methyl-pyrazole-4-sulfonamide (30 mg, 0.07001 mmol) in anhydrous DMF. To the reaction was added 1-bromoethylbenzene (approximately 14.25 mg, 10.51 μL, 0.07701 mmol) and TEA (approximately 21.25 mg, 29.27 μL, 0.2100 mmol). The reaction mixture was allowed to stir at 60° C. for 1 hour. The crude was filtered and purified by reverse phase HPLC (HCl, 15-75% ACN-H₂O) to give N-[4-(2,6-dimethylphenyl)-6-[1-(1-phenylethyl)pyrrolidin-3-yl]oxy-pyrimidin-2-yl]-1-methyl-pyrazole-4-sulfonamide (9.3 mg, 24%). ESI-MS m/z calc. 532.22565, found 533.0 (M+1)⁺; Retention time: 1.12 minutes; LC method A. ¹H NMR (400 MHz, DMSO-d₆) δ 11.37-10.81 (m, 1H), 8.42-8.25 (m, 1H), 7.88-7.72 (m, 1H), 7.69-7.56 (m, 2H), 7.47 (dt, J=5.1, 3.1 Hz, 2H), 7.31-7.22 (m, 1H), 7.14 (dd, J=7.6, 5.2 Hz, 2H), 6.46 (d, J=27.2 Hz, 1H), 5.58-5.38 (m, 1H), 4.50-4.37 (m, 2H), 4.11-3.98 (m, 1H), 3.87 (s, 3H), 3.72-3.60 (m, 1H), 3.36-3.21 (m, 3H), 2.30-2.21 (m, 1H), 2.19-2.08 (m, 1H), 2.03 (s, 3H), 2.00 (s, 3H).

### Example 265: Preparation of Compound 882

### Step 1: N-[4-(2,6-Dimethylphenyl)-6-[1-[2-(p-tolyl)ethyl]pyrrolidin-3-yl]oxy-pyrimidin-2-yl]-1-methyl-pyrazole-4-sulfonamide

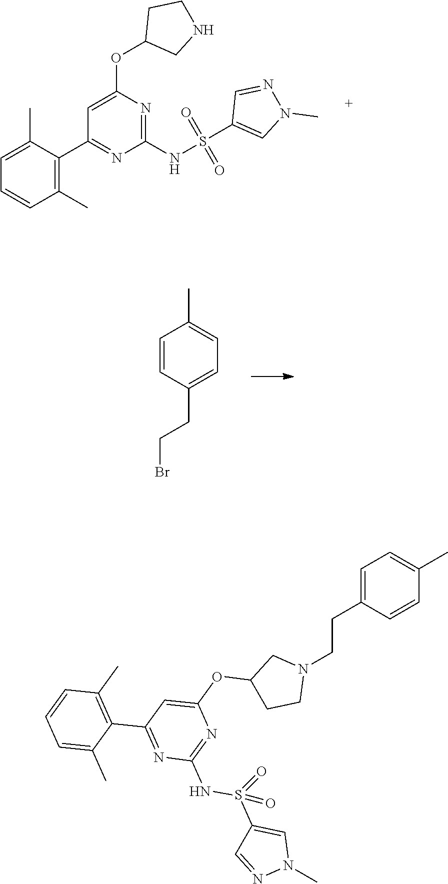

N-[4-(2,6-Dimethylphenyl)-6-pyrrolidin-3-yloxy-pyrimidin-2-yl]-1-methyl-pyrazole-4-sulfonamide (30 mg, 0.07001 mmol) in DMF (0.2 mL) was added to 1-(2-bromoethyl)-4 -methyl-benzene (approximately 27.87 mg, 0.1400 mmol), followed by TEA (approximately 28.33 mg, 39.02 μL, 0.2800 mmol). The mixture was shaken at 60° C. for 1 hour. The reaction mixture was allowed to cool to room temperature, filtered and purified by reverse phase HPLC to give N-[4-(2,6-dimethylphenyl)-6-[1-[2-(p-tolyl)ethyl]pyrrolidin-3-yl]oxy-pyrimidin-2-yl]-1 -methyl-pyrazole-4-sulfonamide (7.2 mg). ESI-MS m/z calc. 546.24133, found 547.0 (M+1)⁺; Retention time: 1.27 minutes; LC method A. ¹H NMR (400 MHz, DMSO-d₆) δ 11.50-10.81 (m, 1H), 8.47-8.26 (m, 1H), 7.87-7.73 (m, 1H), 7.30-7.22 (m, 1H), 7.18-7.13 (m, 5H), 6.54-6.34 (m, 1H), 5.59-5.37 (m, 1H), 4.34-4.18 (m, 1H), 3.88 (d, J=13.4 Hz, 3H), 3.75 (ddd, J=37.7, 11.7, 5.0 Hz, 2H), 3.66-3.57 (m, 1H), 3.34-3.24 (m, 2H), 3.10-2.92 (m, 2H), 2.70-2.59 (m, 1H), 2.42 (dd, J=13.2, 6.1 Hz, 1H), 2.28 (d, J=4.3 Hz, 3H), 2.25-2.10 (m, 1H), 2.06-1.98 (m, 6H).

### Example 266: Preparation of Compound 883

### Step 1: N-[4-(1-benzylpyrrolidin-3-yl)oxy-6-(2,6-dimethylphenyl)pyrimidin-2-yl]-1 -methyl-pyrazole-4-sulfonamide

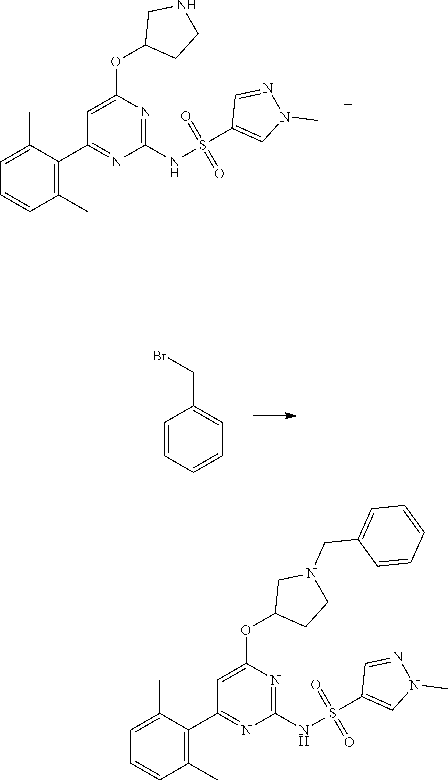 [SMILES: Cc1cccc(C)c1-c1cc(OC2CCNC2)nc(NS(=O)(=O)c2cnn(C)c2)n1 ; BrCc1ccccc1 ; Cc1cccc(C)c1-c1cc(OC2CCN(Cc3ccccc3)C2)nc(NS(=O)(=O)c2cnn(C)c2)n1]

To a vial equipped with magnetic stir bar was added N-[4-(2,6-dimethylphenyl)-6-pyrrolidin-3-yloxy-pyrimidin-2-yl]-1-methyl-pyrazole-4-sulfonamide (30 mg, 0.07001 mmol) in anhydrous DMF. To the reaction solution was added bromomethylbenzene (approximately 13.17 mg, 9.159 μL, 0.07701 mmol) and TEA (approximately 21.25 mg, 29.27 μL, 0.2100 mmol). The reaction mixture was allowed to stir at 60° C. for 1 hour. The crude was filtered and purified by reverse phase HPLC (HCl, 15-75% ACN-H₂O) to give. N-[4-(1-benzylpyrrolidin-3-yl)oxy-6-(2,6-dimethylphenyl)pyrimidin-2-yl]-1-methyl-pyrazole-4-sulfonamide (5.17.3 mg, 19%). ESI-MS m/z calc. 518.21, found 519.0 (M+1)⁺; Retention time: 1.09 minutes; LC method A. ¹H NMR (400 MHz, DMSO-d₆) δ 11.37-10.81 (m, 1H), 8.42-8.25 (m, 1H), 7.88-7.72 (m, 1H), 7.69-7.56 (m, 2H), 7.47 (dt, J=5.1, 3.1 Hz, 2H), 7.31-7.22 (m, 1H), 7.14 (dd, J=7.6, 5.2 Hz, 2H), 6.46 (d, J=27.2 Hz, 1H), 5.58-5.38 (m, 1H), 4.50-4.37 (m, 2H), 4.11-3.98(m, 1H), 3.87 (s, 3H), 3.72-3.60 (m, 1H), 3.36-3.21 (m, 3H), 2.30-2.21 (m, 1H), 2.19-2.08 (m, 1H), 2.03 (s, 3H), 2.00 (s, 3H).

### Example 267: Preparation of Compound 884

### Step 1: N-[4-(2,6-Dimethylphenyl)-6-[1-[[4-(trifluoromethyl)phenyl]methyl]pyrrolidin-3-yl]oxy-pyrimidin-2-yl]-1-methyl-pyrazole-4-sulfonamide

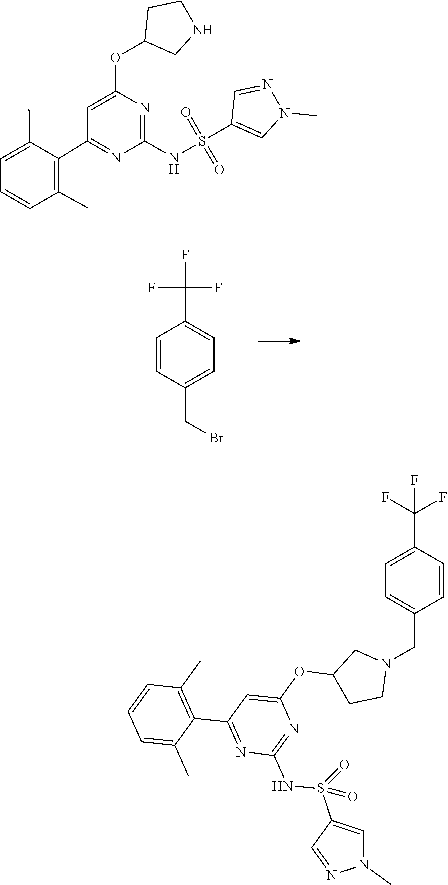 [SMILES: Cc1cccc(C)c1-c1cc(OC2CCNC2)nc(NS(=O)(=O)c2cnn(C)c2)n1 ; FC(F)(F)c1ccc(CBr)cc1 ; Cc1cccc(C)c1-c1cc(OC2CCN(Cc3ccc(C(F)(F)F)cc3)C2)nc(NS(=O)(=O)c2cnn(C)c2)n1]

N-[4-(2,6-Dimethylphenyl)-6-pyrrolidin-3-yloxy-pyrimidin-2-yl]-1-methyl-pyrazole-4-sulfonamide (30 mg, 0.07001 mmol) in DMF (0.2 mL) was added to 1-(bromomethyl)-4-(trifluoromethyl)benzene (approximately 33.46 mg, 0.1400 mmol), followed by TEA (approximately 28.33 mg, 39.02 μL, 0.2800 mmol). The mixture was shaken at 60° C. for 1 hour. The reaction mixture was allowed to cool to room temperature, filtered and purified by reverse phase HPLC to give N-[4-(2,6-dimethylphenyl)-6-[1-[[4-(trifluoromethyl)phenyl]methyl]pyrrolidin-3-yl]oxy-pyrimidin-2-yl]-1-methyl-pyrazole-4-sulfonamide (11.4 mg). ESI-MS m/z calc. 586.1974, found 587.0 (M+1)⁺; Retention time: 1.28 minutes; LC method A. ¹H NMR (400 MHz, DMSO-d₆) δ 12.00-11.17 (m, 1H), 8.51-8.26 (m, 1H), 7.93-7.75 (m, 4H), 7.43-7.02 (m, 3H), 6.71-6.30 (m, 1H), 5.69-5.32 (m, 1H), 4.56 (s, 2H), 4.10 (dd, J=13.1, 6.6 Hz, 1H), 3.87 (d, J=5.1 Hz, 3H), 3.78-3.62 (m, 2H), 2.79 -2.62 (m, 2H), 2.35-2.15 (m, 2H), 2.14-1.90 (m, 6H).

### Example 268: Characterization of Compound 885-912

The compounds in the following tables were prepared in a manner analogous to that described above using commercially available reagents and intermediates described herein.

| | | LCMS | | | |
|---|---|---|---|---|---|
| | | Room | | | |
| Cmpd | | Temperature | Calc. | | LCMS |
| No. | Structure | (min) | mass | M + 1 | Method |
| 885 | 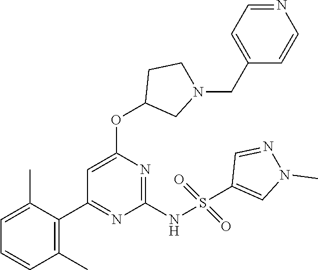 [SMILES: Cc1cccc(C)c1-c1cc(OC2CCN(Cc3ccncc3)C2)nc(NS(=O)(=O)c2cnn(C)c2)n1] | 0.76 | 519.205 | 520 | A |
| 886 | 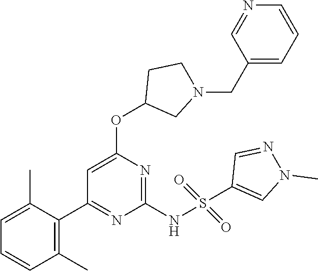 [SMILES: Cc1cccc(C)c1-c1cc(OC2CCN(Cc3cccnc3)C2)nc(NS(=O)(=O)c2cnn(C)c2)n1] | 0.78 | 519.205 | 520 | A |
| 887 | 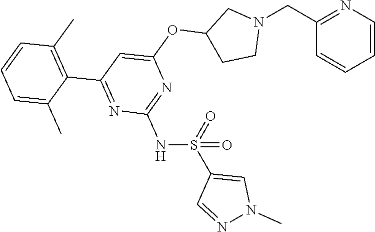 [SMILES: Cc1cccc(C)c1-c1cc(OC2CCN(Cc3ccccn3)C2)nc(NS(=O)(=O)c2cnn(C)c2)n1] | 0.97 | 519.205 | 520 | A |
| 888 | 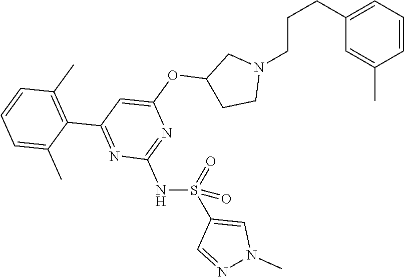 [SMILES: Cc1cccc(CCCN2CCC(Oc3cc(-c4c(C)cccc4C)nc(NS(=O)(=O)c4cnn(C)c4)n3)C2)c1] | 1.25 | 560.257 | 560 | A |
| 889 | 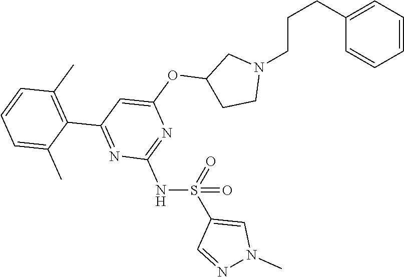 [SMILES: Cc1cccc(C)c1-c1cc(OC2CCN(CCCc3ccccc3)C2)nc(NS(=O)(=O)c2cnn(C)c2)n1] | 1.34 | 546.241 | 547 | A |
| 890 | 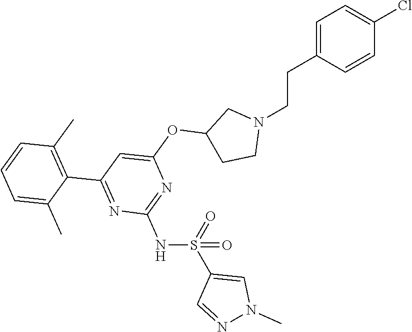 [SMILES: Cc1cccc(C)c1-c1cc(OC2CCN(CCc3ccc(Cl)cc3)C2)nc(NS(=O)(=O)c2cnn(C)c2)n1] | 1.3 | 566.187 | 567 | A |
| 891 | 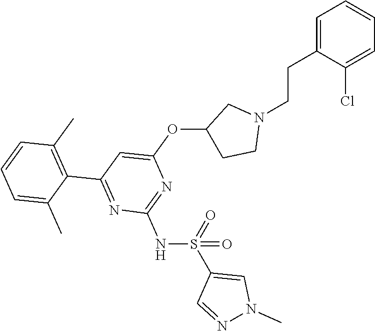 [SMILES: Cc1cccc(C)c1-c1cc(OC2CCN(CCc3ccccc3Cl)C2)nc(NS(=O)(=O)c2cnn(C)c2)n1] | 1.23 | 566.187 | 568 | A |
| 892 | 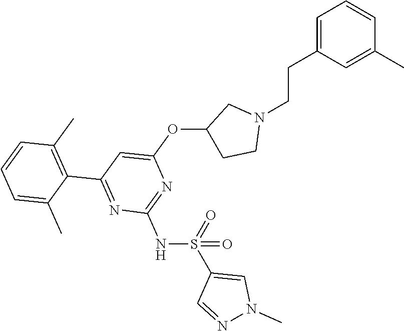 [SMILES: Cc1cccc(CCN2CCC(Oc3cc(-c4c(C)cccc4C)nc(NS(=O)(=O)c4cnn(C)c4)n3)C2)c1] | 1.26 | 546.241 | 546 | A |
| 893 | 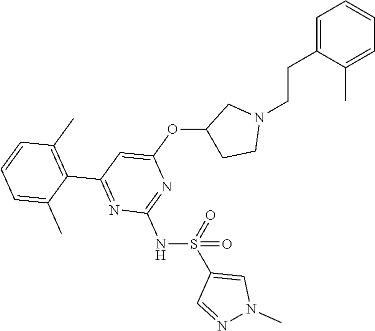 [SMILES: Cc1ccccc1CCN1CCC(Oc2cc(-c3c(C)cccc3C)nc(NS(=O)(=O)c3cnn(C)c3)n2)C1] | 1.26 | 546.241 | 547 | A |
| 894 | 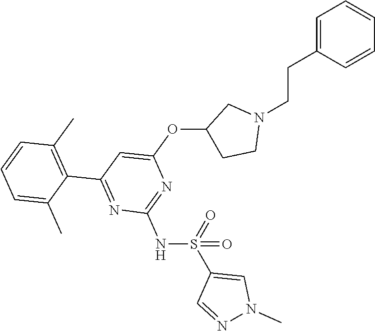 [SMILES: Cc1cccc(C)c1-c1cc(OC2CCN(CCc3ccccc3)C2)nc(NS(=O)(=O)c2cnn(C)c2)n1] | 1.18 | 532.226 | 533 | A |
| 895 | 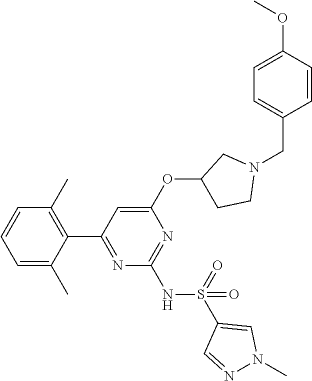 [SMILES: COc1ccc(CN2CCC(Oc3cc(-c4c(C)cccc4C)nc(NS(=O)(=O)c4cnn(C)c4)n3)C2)cc1] | 1.13 | 548.221 | 549 | A |
| 896 | 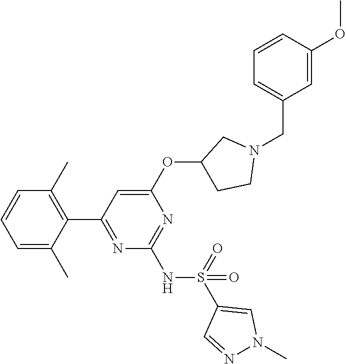 [SMILES: COc1cccc(CN2CCC(Oc3cc(-c4c(C)cccc4C)nc(NS(=O)(=O)c4cnn(C)c4)n3)C2)c1] | 1.14 | 548.221 | 549 | A |
| 897 | 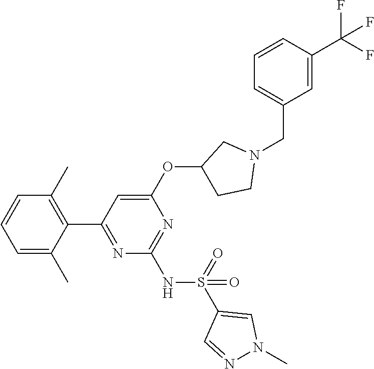 [SMILES: Cc1cccc(C)c1-c1cc(OC2CCN(Cc3cccc(C(F)(F)F)c3)C2)nc(NS(=O)(=O)c2cnn(C)c2)n1] | 1.27 | 586.197 | 587 | A |
| 898 | 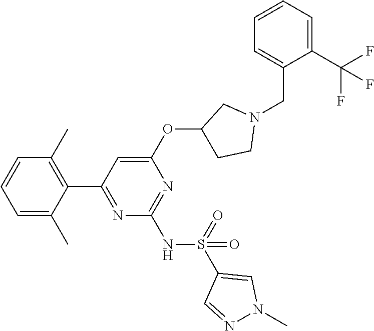 [SMILES: Cc1cccc(C)c1-c1cc(OC2CCN(Cc3ccccc3C(F)(F)F)C2)nc(NS(=O)(=O)c2cnn(C)c2)n1] | 1.25 | 586.197 | 587 | A |
| 899 | 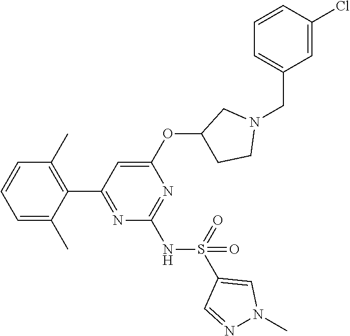 [SMILES: Cc1cccc(C)c1-c1cc(OC2CCN(Cc3cccc(Cl)c3)C2)nc(NS(=O)(=O)c2cnn(C)c2)n1] | 1.17 | 552.171 | 553 | A |
| 900 | 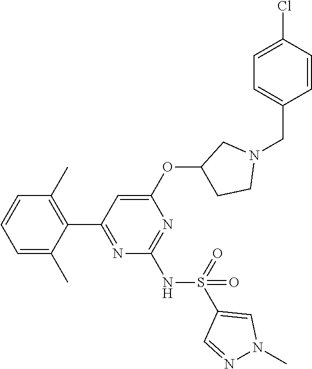 [SMILES: Cc1cccc(C)c1-c1cc(OC2CCN(Cc3ccc(Cl)cc3)C2)nc(NS(=O)(=O)c2cnn(C)c2)n1] | 1.19 | 552.171 | 553 | A |
| 901 | 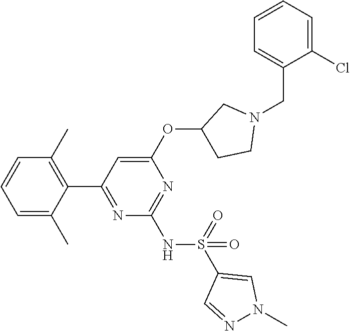 [SMILES: Cc1cccc(C)c1-c1cc(OC2CCN(Cc3ccccc3Cl)C2)nc(NS(=O)(=O)c2cnn(C)c2)n1] | 1.15 | 552.171 | 553 | A |
| 902 | 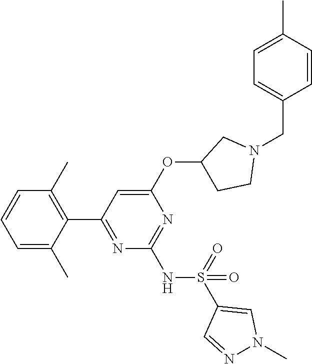 [SMILES: Cc1ccc(CN2CCC(Oc3cc(-c4c(C)cccc4C)nc(NS(=O)(=O)c4cnn(C)c4)n3)C2)cc1] | 1.18 | 532.226 | 533 | A |
| 903 | 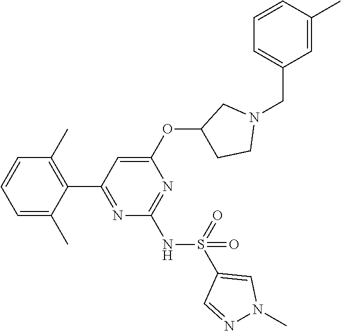 [SMILES: Cc1cccc(CN2CCC(Oc3cc(-c4c(C)cccc4C)nc(NS(=O)(=O)c4cnn(C)c4)n3)C2)c1] | 1.17 | 532.226 | 533 | A |
| 904 | 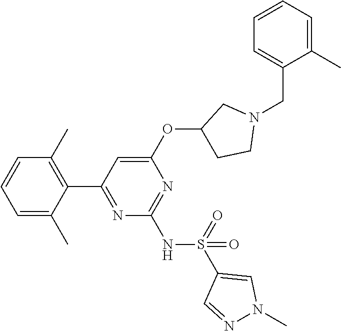 [SMILES: Cc1ccccc1CN1CCC(Oc2cc(-c3c(C)cccc3C)nc(NS(=O)(=O)c3cnn(C)c3)n2)C1] | 1.16 | 532.226 | 533 | A |
| 905 | 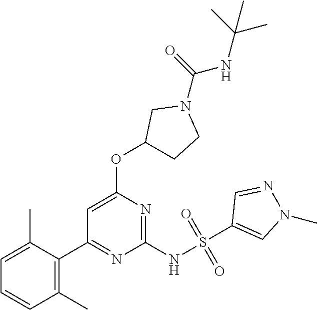 [SMILES: Cc1cccc(C)c1-c1cc(OC2CCN(C(=O)NC(C)(C)C)C2)nc(NS(=O)(=O)c2cnn(C)c2)n1] | 1.44 | 527.231 | 528 | A |
| 906 | 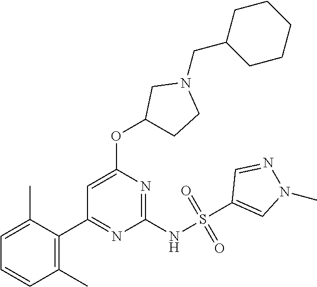 [SMILES: Cc1cccc(C)c1-c1cc(OC2CCN(CC3CCCCC3)C2)nc(NS(=O)(=O)c2cnn(C)c2)n1] | 1.2 | 524.257 | 525 | A |
| 907 | 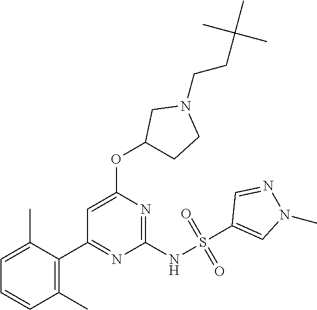 [SMILES: Cc1cccc(C)c1-c1cc(OC2CCN(CCC(C)(C)C)C2)nc(NS(=O)(=O)c2cnn(C)c2)n1] | 1.19 | 512.257 | 513 | A |
| 908 | 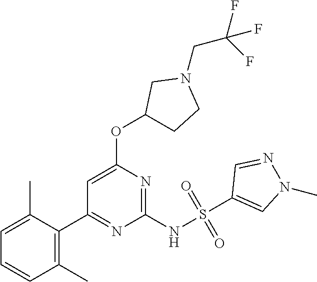 [SMILES: Cc1cccc(C)c1-c1cc(OC2CCN(CC(F)(F)F)C2)nc(NS(=O)(=O)c2cnn(C)c2)n1] | 1.3 | 510.166 | 511 | A |
| 909 | 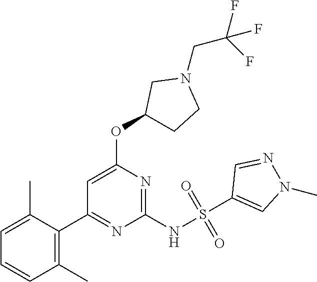 [SMILES: Cc1cccc(C)c1-c1cc(O[C@@H]2CCN(CC(F)(F)F)C2)nc(NS(=O)(=O)c2cnn(C)c2)n1] | 1.59 | 510.166 | 511 | A |
| 910 | 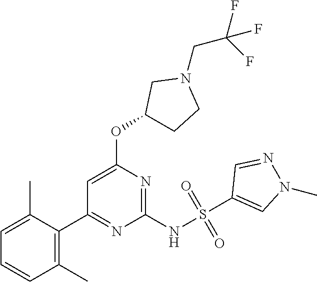 [SMILES: Cc1cccc(C)c1-c1cc(O[C@H]2CCN(CC(F)(F)F)C2)nc(NS(=O)(=O)c2cnn(C)c2)n1] | 1.29 | 510.166 | 511 | A |
| 911 | 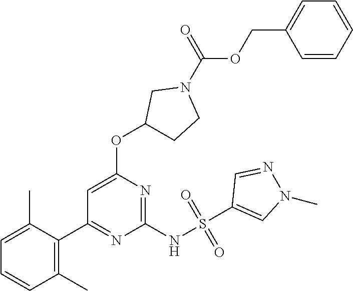 [SMILES: Cc1cccc(C)c1-c1cc(OC2CCN(C(=O)OCc3ccccc3)C2)nc(NS(=O)(=O)c2cnn(C)c2)n1] | 1.68 | 562.2 | 563 | A |
| 912 | 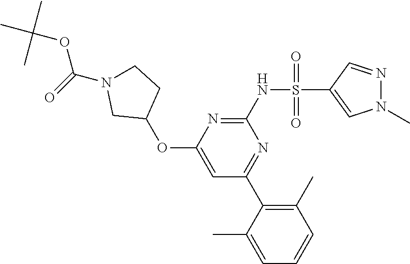 [SMILES: Cc1cccc(C)c1-c1cc(OC2CCN(C(=O)OC(C)(C)C)C2)nc(NS(=O)(=O)c2cnn(C)c2)n1] | 1.68 | 528.215 | 529 | A |

| Compound | |
|---|---|
| number | NMR |
| 887 | ¹H NMR (400 MHz, DMSO-d₆) δ 11.38 (s, 1H), 8.67 (s, 1H), 8.33 (s, 1H), 7.95 |
| | (t, J = 8.2 Hz, 1H), 7.78 (s, 1H), 7.67 (d, J = 7.7 Hz, 1H), 7.54 - 7.47 (m, 1H), |
| | 7.28 - 7.22 (m, 1H), 7.13 (d, J = 7.6 Hz, 2H), 6.44 (s, 1H), 5.49 (s, 1H), 4.63 (s, |
| | 2H), 3.86 (s, 4H), 3.55 (s, 4H), 2.24 (s, 2H), 2.02 (s, 8H). |
| 888 | ¹H NMR (400 MHz, DMSO-d₆) δ 11.70 (s, 1H), 8.36 (d, J = 55.3 Hz, 1H), 7.79 |
| | (d, J = 10.1 Hz, 1H), 7.28 - 7.22 (m, 1H), 7.21 - 7.16 (m, 1H), 7.14 (d, J = 7.5 |
| | Hz, 2H), 7.03 (dd, J = 17.3, 8.2 Hz, 3H), 6.44 (s, 1H), 5.43 (d, J = 40.0 Hz, 1H), |
| | 4.24 (d, J = 9.2 Hz, 1H), 3.86 (s, 3H), 3.72 (d, J = 34.9 Hz, 2H), 3.51 (s, 1H), |
| | 3.19 (d, J = 18.9 Hz, 4H), 2.62 (t, J = 7.6 Hz, 3H), 2.44 (s, 1H), 2.29 (d, J = 8.1 |
| | Hz, 3H), 2.22 (s, 1H), 2.02 (s, 8H). |
| 889 | ¹H NMR (400 MHz, DMSO-d₆) δ 11.71 (s, 1H), 11.19 (s, 1H), 10.62 (s, 1H), |
| | 8.36 (d, J = 48.1 Hz, 1H), 7.80 (d, J = 9.9 Hz, 1H), 7.32 (q, J = 7.8 Hz, 2H), |
| | 7.27 - 7.18 (m, 4H), 7.14 (d, J = 7.5 Hz, 2H), 6.42 (s, 1H), 5.43 (d, J = 41.7 Hz, |
| | 1H), 4.23 (s, 1H), 3.87 (d, J = 13.9 Hz, 3H), 3.74 (d, J = 31.0 Hz, 2H), 3.52 (s, |
| | 1H), 3.17 (s, 5H), 2.67 (t, J = 7.6 Hz, 3H), 2.41 (s, 1H), 2.24 (s, 1H), 2.01 (s, |
| | 9H). |
| 892 | ¹H NMR (400 MHz, DMSO-d₆) δ 11.36 (d, J = 200.8 Hz, 1H), 8.37 (d, J = 49.6 |
| | Hz, 1H), 7.80 (d, J = 12.1 Hz, 1H), 7.25 (s, 2H), 7.16 - 7.06 (m, 4H), 6.45 (d, J = |
| | 14.4 Hz, 1H), 5.47 (d, J = 42.1 Hz, 1H), 4.28 (s, 1H), 3.97 - 3.69 (m, 6H), |
| | 3.29 (s, 2H), 3.01 (s, 2H), 2.65 (dd, J = 14.4, 7.8 Hz, 1H), 2.47 - 2.39 (m, 1H), |
| | 2.30 (d, J = 6.6 Hz, 3H), 2.21 (d, J = 5.6 Hz, 1H), 2.02 (s, 6H). |
| 893 | ¹H NMR (400 MHz, DMSO-d₆) δ 11.28 (d, J = 241.2 Hz, 1H), 8.37 (d, J = 48.4 |
| | Hz, 1H), 7.81 (d, J = 11.0 Hz, 1H), 7.26 - 7.13 (m, 7H), 6.49 (d, J = 17.0 Hz, |
| | 1H), 5.48 (d, J = 47.1 Hz, 1H), 4.29 (s, 1H), 3.88 (d, J = 15.4 Hz, 4H), 3.77 (s, |
| | 1H), 3.62 (tt, J = 11.9, 5.6 Hz, 1H), 3.33 (s, 3H), 3.17 (s, 1H), 3.01 (s, 2H), 2.68 |
| | (s, 1H), 2.33 (d, J = 7.5 Hz, 3H), 2.21 (d, J = 29.6 Hz, 1H), 2.02 (s, 6H). |
| 894 | 1H NMR (400 MHz, DMSO-d₆) δ 12.00 - 11.17 (m, 1H), 8.51 - 8.26 (m, 1H), |
| | 7.93 - 7.75 (m, 4H), 7.43 - 7.02 (m, 3H), 6.71 - 6.30 (m, 1H), 5.69 - 5.32 (m, |
| | 1H), 4.56 (d, J = 6.9 Hz, 2H), 4.10 (dd, J = 13.1, 6.6 Hz, 1H), 3.87 (d, J = 5.1 |
| | Hz, 3H), 3.78 - 3.62 (m, 2H), 2.79 - 2.62 (m, 2H), 2.35 - 2.15 (m, 2H), 2.14 - |
| | 1.90 (m, 6H). |
| 896 | ¹H NMR (400 MHz, DMSO-d₆) δ 11.49 (d, J = 214.2 Hz, 1H), 8.35 (d, J = 46.0 |
| | Hz, 1H), 7.84 - 7.73 (m, 1H), 7.40 - 7.22 (m, 3H), 7.20 - 7.10 (m, 3H), 7.01 (t, |
| | J = 8.9 Hz, 1H), 6.45 (d, J = 31.1 Hz, 1H), 5.47 (d, J = 29.4 Hz, 1H), 4.39 (s, |
| | 2H), 4.06 (s, 1H), 3.87 (d, J = 6.5 Hz, 3H), 3.80 (d, J = 2.3 Hz, 3H), 3.31 (s, |
| | 2H), 2.27 - 2.11 (m, 1H), 2.02 (d, J = 8.6 Hz, 6H). |
| 897 | ¹H NMR (400 MHz, DMSO-d₆) δ 11.51 (d, J = 220.3 Hz, 2H), 8.34 (d, J = 32.7 |
| | Hz, 1H), 8.08 (d, J = 11.6 Hz, 1H), 7.97 (s, 1H), 7.87 - 7.67 (m, 3H), 7.20 (d, J = |
| | 46.0 Hz, 3H), 6.43 (d, J = 12.8 Hz, 1H), 5.48 (d, J = 52.2 Hz, 1H), 4.56 (s, |
| | 2H), 4.11 (s, 1H), 3.87 (s, 3H), 2.16 (s, 2H), 2.03 (s, 6H). |
| 898 | ¹H NMR (400 MHz, DMSO-d₆) δ 11.50 (d, J = 236.8 Hz, 1H), 8.49 - 8.11 (m, |
| | 2H), 7.93 - 7.74 (m, 3H), 7.68 (t, J = 7.6 Hz, 1H), 7.25 (t, J = 7.5 Hz, 1H), 7.14 |
| | (d, J = 7.5 Hz, 2H), 6.42 (s, 1H), 5.52 (d, J = 39.2 Hz, 1H), 4.64 (d, J = 13.1 |
| | Hz, 2H), 4.20 (s, 1H), 3.87 (s, 4H), 3.63 (s, 1H), 2.22 (d, J = 48.7 Hz, 1H), 2.03 |
| | (s, 6H). |
| 899 | ¹H NMR (400 MHz, DMSO-d₆) δ 11.47 (d, J = 219.6 Hz, 1H), 8.34 (d, J = 37.2 |
| | Hz, 1H), 7.82 - 7.73 (m, 2H), 7.62 (t, J = 6.4 Hz, 1H), 7.56 - 7.46 (m, 2H), 7.38 - |
| | 7.22 (m, 2H), 7.14 (dd, J = 7.5, 4.4 Hz, 2H), 6.45 (d, J = 30.6 Hz, 1H), 5.46 |
| | (d, J = 42.9 Hz, 1H), 4.51 - 4.40 (m, 2H), 4.08 (dt, J = 12.5, 5.9 Hz, 1H), 3.88 |
| | (d, J = 3.5 Hz, 3H), 3.37 - 3.27 (m, 2H), 2.72 - 2.60 (m, 1H), 2.20 (ddd, J = |
| | 28.6, 10.5, 5.5 Hz, 1H), 2.02 (d, J = 8.5 Hz, 6H). |
| 900 | ¹H NMR (400 MHz, DMSO-d₆) δ 11.50 (d, J = 220.3 Hz, 1H), 8.35 (d, J = 38.4 |
| | Hz, 1H), 7.78 (d, J = 17.7 Hz, 1H), 7.72 - 7.64 (m, 2H), 7.54 (t, J = 8.2 Hz, 2H), |
| | 7.36 (q, J = 8.4 Hz, 1H), 7.29 - 7.22 (m, 1H), 7.19 - 7.11 (m, 2H), 6.44 (d, J = |
| | 29.5 Hz, 1H), 5.45 (d, J = 44.6 Hz, 1H), 4.50 - 4.40 (m, 2H), 4.13 - 4.02 (m, |
| | 1H), 3.88 (d, J = 4.0 Hz, 3H), 3.31 (s, 2H), 2.68 (s, 1H), 2.26 (s, 1H), 2.02 (d, J = |
| | 7.9 Hz, 7H). |
| 901 | ¹H NMR (400 MHz, DMSO-d₆) δ 11.38 (d, J = 220.4 Hz, 1H), 8.36 (d, J = 40.0 |
| | Hz, 1H), 7.90 (d, J = 24.5 Hz, 1H), 7.79 (d, J = 17.9 Hz, 1H), 7.63 - 7.23 (m, |
| | 5H), 7.14 (d, J = 7.6 Hz, 2H), 6.44 (d, J = 21.2 Hz, 1H), 5.50 (d, J = 35.9 Hz, |
| | 1H), 4.59 (d, J = 17.6 Hz, 2H), 4.12 (d, J = 6.1 Hz, 1H), 3.87 (s, 3H), 3.77 (s, |
| | 1H), 3.40 (s, 3H), 2.71 (s, 1H), 2.30 - 2.12 (m, 1H), 2.02 (d, J = 6.2 Hz, 6H). |
| 902 | ¹H NMR (400 MHz, DMSO-d₆) δ 11.31 (d, J = 215.8 Hz, 1H), 8.34 (d, J = 38.6 |
| | Hz, 1H), 7.78 (d, J = 15.8 Hz, 1H), 7.50 (d, J = 7.8 Hz, 2H), 7.32 - 7.21 (m, |
| | 3H), 7.18 - 7.10 (m, 2H), 6.44 (d, J = 28.3 Hz, 1H), 5.45 (d, J = 32.6 Hz, 1H), |
| | 4.37 (d, J = 5.2 Hz, 2H), 4.02 (d, J = 13.2 Hz, 1H), 3.87 (d, J = 2.6 Hz, 3H), |
| | 2.33 (d, J = 4.3 Hz, 3H), 2.18 (d, J = 44.8 Hz, 2H), 2.02 (d, J = 9.9 Hz, 6H). |
| 903 | ¹H NMR (400 MHz, DMSO-d₆) δ 11.14 (d, J = 185.9 Hz, 1H), 8.33 (d, J = 34.7 |
| | Hz, 1H), 7.78 (d, J = 15.1 Hz, 1H), 7.41 (d, J = 6.7 Hz, 2H), 7.38 - 7.31 (m, |
| | 1H), 7.29 - 7.22 (m, 2H), 7.18 - 7.09 (m, 2H), 6.45 (d, J = 30.6 Hz, 1H), 5.46 |
| | (d, J = 39.6 Hz, 1H), 4.39 (d, J = 5.7 Hz, 2H), 4.05 (d, J = 6.6 Hz, 1H), 3.87 (s, |
| | 3H), 3.69 (s, 1H), 3.35 - 3.27 (m, 2H), 2.73 - 2.61 (m, 1H), 2.34 (d, J = 8.7 Hz, |
| | 3H), 2.28 - 2.07 (m, 2H), 2.02 (d, J = 9.8 Hz, 6H). |
| 904 | ¹H NMR (400 MHz, DMSO-d₆) δ 11.12 (d, J = 228.4 Hz, 1H), 8.38 (d, J = 55.0 |
| | Hz, 1H), 7.79 (d, J = 18.1 Hz, 1H), 7.64 (t, J = 6.4 Hz, 1H), 7.40 - 7.21 (m, 4H), |
| | 7.19 - 7.09 (m, 2H), 6.45 (d, J = 26.6 Hz, 1H), 5.48 (d, J = 33.6 Hz, 1H), 4.46 |
| | (d, J = 5.4 Hz, 2H), 4.16 (d, J = 12.8 Hz, 1H), 3.87 (d, J = 4.4 Hz, 3H), 3.38 (s, |
| | 2H), 2.64 (d, J = 51.3 Hz, 1H), 2.45 (d, J = 5.4 Hz, 3H), 2.26 (s, 1H), 2.02 (d, J = |
| | 8.6 Hz, 6H). |
| 905 | ¹H NMR (400 MHz, DMSO-d₆) δ 8.27 (s, 1H), 7.74 (s, 1H), 7.29 - 7.19 (m, 1H), |
| | 7.12 (d, J = 7.6 Hz, 2H), 6.40 (s, 1H), 5.45 (s, 1H), 5.32 (s, 1H), 3.86 (s, 3H), |
| | 3.58 (d, J = 12.0 Hz, 1H), 3.29 (d, J = 11.9 Hz, 1H), 2.20 (s, 1H), 2.02 (s,7H), |
| | 1.25 (s, 10H). |
| 906 | ¹H NMR (400 MHz, DMSO-d₆) δ 11.70 (s, 1H), 9.78 (d, J = 28.2 Hz, 1H), 8.32 |
| | (s, 1H), 7.81 (s, 1H), 7.25 (s, 1H), 7.14 (d, J = 7.4 Hz, 2H), 6.49 (s, 1H), 5.45 |
| | (d, J = 35.0 Hz, 1H), 3.87 (s, 5H), 3.06 (s, 2H), 2.68 (p, J = 1.9 Hz, 1H), 2.01 |
| | (s, 7H), 1.72 (s, 6H), 1.33 - 1.10 (m, 3H), 0.96 (d, J = 10.0 Hz, 2H). |
| 907 | ¹H NMR (400 MHz, DMSO-d₆) δ 9.99 (s, 1H), 8.30 (s, 1H), 7.81 (s, 1H), 7.25 |
| | (s, 1H), 7.14 (d, J = 7.4 Hz, 2H), 6.49 (s, 1H), 5.45 (s, 1H), 3.87 (s, 3H), 3.15 |
| | (s, 2H), 2.00 (s, 7H), 1.53 (s, 2H), 0.92 (s, 9H). |
| 908 | ¹H NMR (400 MHz, DMSO-d₆) δ 8.29 (s, 1H), 7.75 (s, 1H), 7.27 - 7.20 (m, 1H), |
| | 7.13 (d, J = 7.6 Hz, 2H), 6.42 (s, 1H), 5.37 (s, 1H), 3.86 (s, 4H), 3.19 (s, 3H), |
| | 2.43 (s, 1H), 2.02 (s, 8H). |
| 911 | ¹H NMR (400 MHz, DMSO-d₆) δ 8.25 (d, J = 6.6 Hz, 1H), 7.74 (s, 1H), 7.36 |
| | (d, J = 11.7 Hz, 5H), 7.26 - 7.20 (m, 1H), 7.12 (d, J = 7.6 Hz, 2H), 6.37 (s, 1H), |
| | 5.46 (d, J = 12.9 Hz, 1H), 5.08 (s, 2H), 3.82 (d, J = 21.3 Hz, 5H), 2.24 (s, 1H), |
| | 2.01 (s, 8H). |
| 912 | ¹H NMR (400 MHz, DMSO-d₆) δ 8.27 (s, 1H), 7.75 (s, 1H), 7.28 - 7.20 (m, 1H), |
| | 7.12 (d, J = 7.6 Hz, 2H), 6.40 (s, 1H), 5.44 (s, 1H), 3.86 (s, 3H), 3.64 (s, 2H), |
| | 3.34 (d, J = 12.8 Hz, 3H), 2.21 (s, 1H), 2.02 (s, 7H), 1.40 (s, 10H). |

### Example 269: Preparation of Compound 905

### Step 1: N-tert-Butyl-3-[6-(2,6-dimethylphenyl)-2-[(1-methylpyrazol-4-yl)sulfonylamino]pyrimidin-4-yl]oxy-pyrrolidine-1-carboxamide

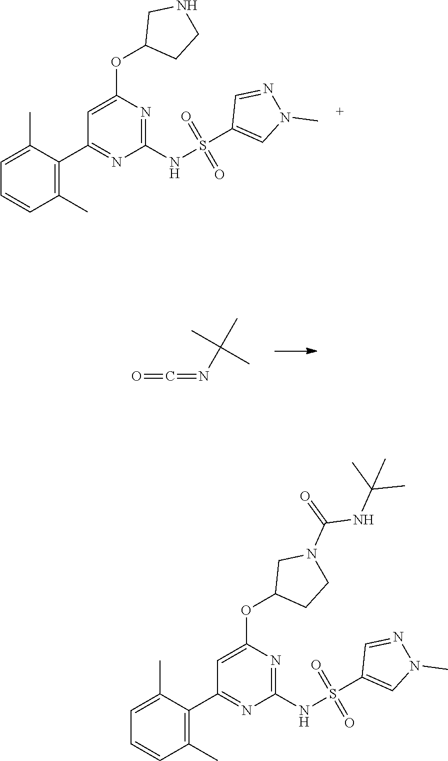

A solution of N-[4-(2,6-dimethylphenyl)-6-pyrrolidin-3-yloxy-pyrimidin-2-yl]-1 -methyl-pyrazole-4-sulfonamide (30 mg, 0.07001 mmol) in THE (600.0 μL) was added 2-isocyanato-2-methyl-propane (8 μL, 0.07005 mmol) and TEA (20 μL, 0.1435 mmol). The mixture was stirred for 2 hours. The reaction mixture was filtered and purified b reverse phase HPLC (HCl modifier, 10-60%0 gradient of ACN in water) to give N-tert-butyl-3-[6-(2,6-dimethylphenyl)-2-[(1-methylpyrazol-4-yl)sulfonylamino]pyrimidin-4-yl] oxy-pyrrolidine-1l-carboxamide (12.7 mg, 340%). ¹H NMR (400 MHz, DMSO-d₆) δ 8.27 (s, 1H), 7.74 (s, 1H), 7.29 -7.19 (m, 1H), 7.12 (d, J=7.6 Hz, 2H), 6.40 (s, 1H), 5.45 (s, 1H), 5.32 (s, 1H), 3.86 (s, 3H), 3.58 (d, J=12.0 Hz, 1H), 3.29 (d, J=11.9 Hz, 1H), 2.20 (s, 1H), 2.02 (s, 7H), 1.25 (s, 10H). ESI-MS m/z calc. 527.23145, found 528.0 (M+1)⁺; Retention time: 1.44 minutes (LC method A).

### Example 270: Preparation of Compound 875

### Step 1: N-[4-[1-(3,3-Dimethylbutanoyl)pyrrolidin-3-yl]oxy-6-(2,6-dimethylphenyl)pyrimidin-2-yl]-1-methyl-pyrazole-4-sulfonamide

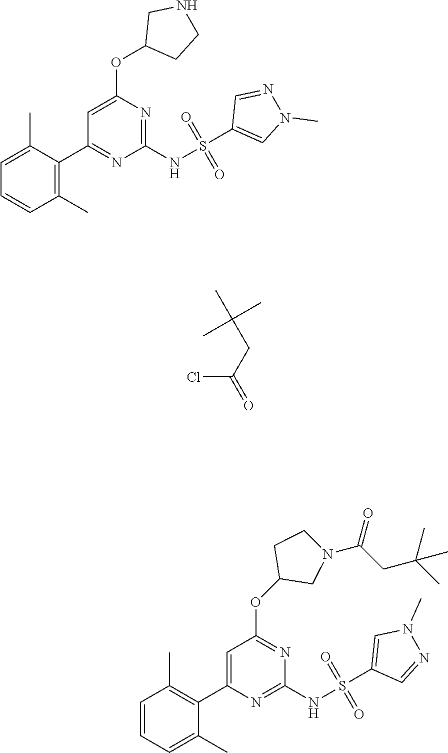

3,3-Dimethylbutanoyl chloride (approximately 14.13 mg, 14.58 μL, 0.1050 mmol) was added to N-[4-(2,6-dimethylphenyl)-6-pyrrolidin-3-yloxy-pyrimidin-2-yl]-1-methyl-pyrazole-4-sulfonamide (30 mg, 0.07001 mmol) and TEA (approximately 28.33 mg, 39.02 μL, 0.2800 mmol) in DCM (1 mL). The mixture was stirred at room temperature. The solvent was evaporated and the crude was dissolved in DMSO, filtered and purified by reverse phase HPLC (HCl modifier, 15-75% gradient of ACN in water) to give N-[4-[1-(3,3-dimethylbutanoyl)pyrrolidin-3-yl]oxy-6-(2,6-dimethylphenyl)pyrimidin-2-yl]-1-methyl-pyrazole-4-sulfonamide (3.6 mg, 9%). ESI-MS m/z calc. 526.2362, found 527.0 (M+1)⁺; Retention time: 1.55 minutes (LC method A). ¹H NMR (400 MHz, DMSO-d₆) δ 8.28 (d, J=4.9 Hz, 1H), 7.76 (d, J=5.8 Hz, 1H), 7.23 (t, J=7.5 Hz, 1H), 7.12 (d, J=7.6 Hz, 2H), 6.38 (d, J=5.4 Hz, 1H), 5.44 (d, J=5.7 Hz, 1H), 3.86 (s, 3H), 3.82 (dd, J=12.2, 4.5 Hz, 1H), 3.52 (d, J=12.9 Hz, 3H), 3.37 (s, 1H), 2.35-2.20 (m, 1H), 2.18 (d, J=2.4 Hz, 1H), 2.13 (s, 1H), 2.07 (d, J=14.4 Hz, 1H), 2.02 (s, 6H), 0.98 (d, J=18.6 Hz, 9H).

### Example 271: Preparation of Compound 913

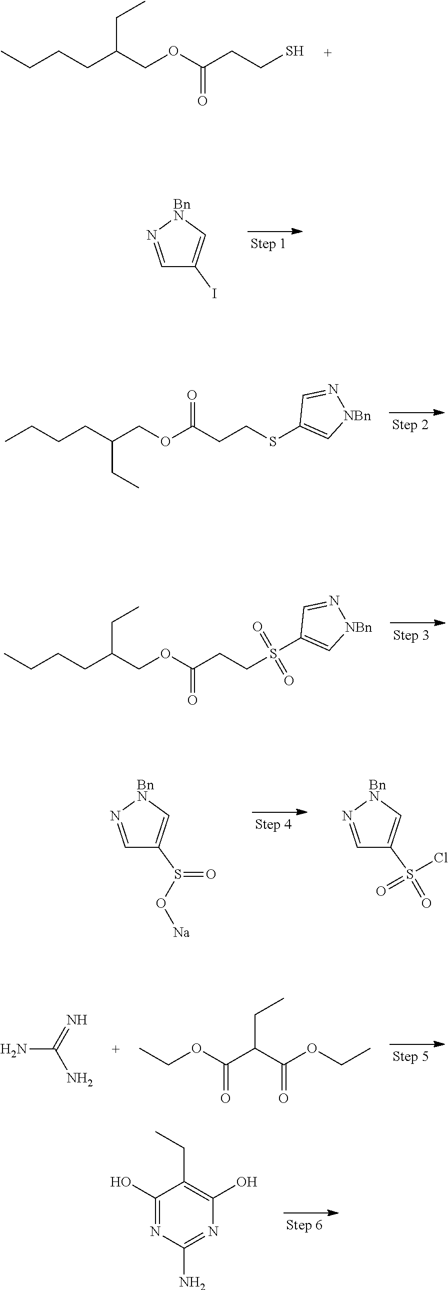

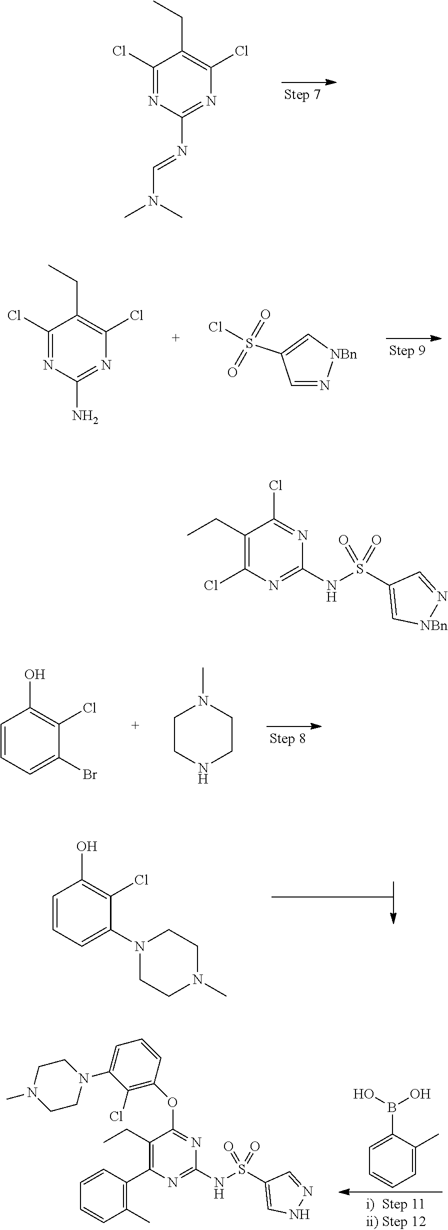

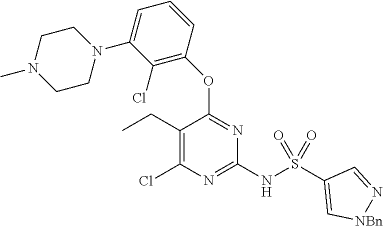 [SMILES: CCc1c(Cl)nc(NS(=O)(=O)c2cnn(Cc3ccccc3)c2)nc1Oc1cccc(N2CCN(C)CC2)c1Cl]

### Step 1: 2-Ethylhexyl 3-(1-benzylpyrazol-4-yl)sulfanylpropanoate

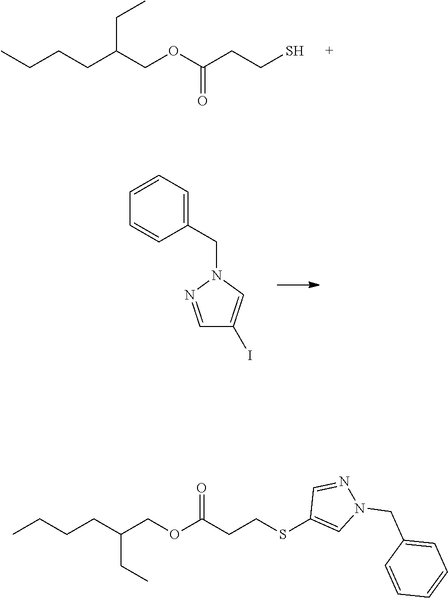 [SMILES: CCCCC(CC)COC(=O)CCS ; Ic1cnn(Cc2ccccc2)c1 ; CCCCC(CC)COC(=O)CCSc1cnn(Cc2ccccc2)c1]

In a sealed tube, a solution of 1-benzyl-4-iodo-pyrazole (500 mg, 1.7600 mmol) and diisopropylethylamine (460.04 mg, 0.62 mL, 3.5595 mmol) in toluene (10 mL) was degassed by bubbling nitrogen for 5 minutes. tris(dibenzylideneacetone)dipalladium(0) (48 mg, 0.0524 mmol), xantphos (61 mg, 0.1054 mmol) and 2-ethylhexyl 3-sulfanylpropanoate (425 mg, 1.9464 mmol) were added, the tube was sealed and the mixture was heated at 110° C. overnight. The reaction mixture was concentrated under reduced pressure. The residue was purified by silica gel chromatography using 0% to 15% of EtOAc in heptanes to afford 2-ethylhexyl 3-(1-benzylpyrazol-4-yl)sulfanylpropanoate (438 mg, 66%) as yellow oil. ¹H NMR (300 MHz, CDCl₃) ppm 0.80-0.96 (m, 6H), 1.18-1.42 (m, 9H), 2.48-2.61 (m, 2H), 2.78-2.91 (m, 2H), 3.94-4.04 (m, 2H), 5.28 (s, 2H), 7.20-7.25 (m, 2H), 7.29-7.40 (m, 3H), 7.43 (s, 1H), 7.55 (s, 1H). ESI-MS m/z calc. 374.2028, found 375.2 (M+1)⁺; Retention time: 2.51 minutes.

### Step 2: 2-Ethylhexyl 3-(1-benzylpyrazol-4-yl)sulfonylpropanoate

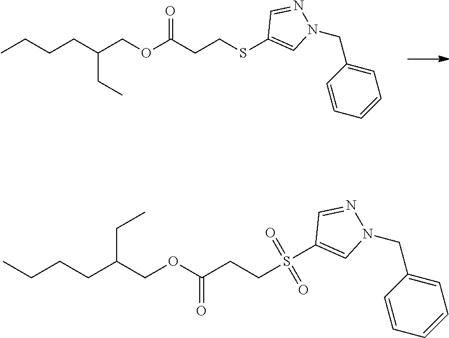 [SMILES: CCCCC(CC)COC(=O)CCSc1cnn(Cc2ccccc2)c1 ; CCCCC(CC)COC(=O)CCS(=O)(=O)c1cnn(Cc2ccccc2)c1]

m-Chloroperbenzoic acid (550 mg, 2.4541 mmol) was slowly added to a solution of 2-ethylhexyl 3-(1-benzylpyrazol-4-yl)sulfanylpropanoate (438 mg, 1.1694 mmol) in dichloromethane (5 mL) at room temperature. The mixture was stirred at this temperature overnight. Ethyl acetate (50 mL) was added and the mixture was washed with an aqueous saturated sodium bicarbonate solution (50 mL) and an aqueous 0.5 M sodium hydroxide solution (2×50 mL). The organic phase was dried over anhydrous sodium sulfate and concentrated under reduced pressure to afford 2-ethylhexyl 3-(1-benzylpyrazol-4-yl)sulfonylpropanoate (409 mg, 86%) as yellowish oil. ¹H NMR (300 MHz, CDCl₃) ppm 0.77-0.95 (m, 6H), 1.15-1.39 (m, 9H), 2.77 (t, J=7.8 Hz, 2H), 3.36-3.51 (m, 2H), 3.96 (dd, J=5.7, 2.5 Hz, 2H), 5.33 (s, 2H), 7.19-7.33 (m, 2H), 7.34-7.43 (m, 3H), 7.80 (s, 1H), 7.86 (s, 1H). ESI-MS m/z calc. 406.1926, found 407.2 (M+1)⁺+; Retention time: 2.29 minutes. The crude compound was used for next step without further purification.

### Step 3: (1-benzylpyrazol-4-yl)sulfinyloxysodium

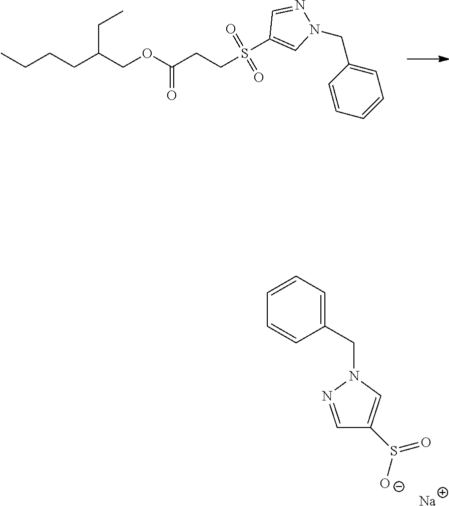 [SMILES: CCCCC(CC)COC(=O)CCS(=O)(=O)c1cnn(Cc2ccccc2)c1 ; O=S([O-])c1cnn(Cc2ccccc2)c1 ; [Na+]]

Sodium methoxide (218 mg, 4.0353 mmol) was added to a solution of 2-ethylhexyl 3-(1-benzylpyrazol-4-yl)sulfonylpropanoate (409 mg, 1.0061 mmol) in tetrahydrofuran (1 mL) and methanol (0.35 mL) at room temperature. The reaction mixture was stirred for 4 hours then the mixture was concentrated under reduced pressure to afford (1-benzylpyrazol-4-yl)sulfinyloxysodium (450 mg, 183%) as beige solid. ESI-MS m/z calc. 221.04, found 223.1 (M+1)⁺+; Retention time: 1.3 minutes. The compound was used in the next step without purification.

### Step 4: 1-benzylpyrazole-4-sulfonyl chloride

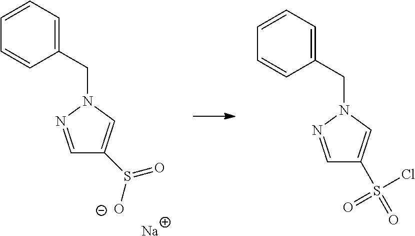 [SMILES: O=S([O-])c1cnn(Cc2ccccc2)c1 ; O=S(=O)(Cl)c1cnn(Cc2ccccc2)c1 ; [Na+]]

The crude (1-benzylpyrazol-4-yl)sulfinyloxysodium (245 mg, 1.0031 mmol) was suspended in dichloromethane (5 mL) then cooled to 0° C. To the mixture was added N-chlorosuccinimide (150 mg, 1.1233 mmol) portionwise under nitrogen atmosphere then the mixture was stirred at room temperature one hour. More dichloromethane (30 mL) was added and the mixture was stirred for one hour at room temperature. The solution was diluted with dichloromethane (50 mL) and filtrated on Celite. The filtrate was washed with water (50 mL) then with brine (50 mL). The solution was dried over sodium sulfate, filtered then concentrated to afford crude 1-benzylpyrazole-4-sulfonyl chloride (227 mg, 71%) as yellowish oil. ¹H NMR (300 MHz, CDCl₃) ppm 5.35 (s, 2H), 7.28-7.37 (m, 2H), 7.39-7.48 (m, 3H), 7.93 (s, 1H), 8.01 (s, 1H). ESI-MS m/z calc. 256.0073, found 237.1 (M-C₁+OH)⁻—; Retention time: 2.81 minutes.

### Step 5: 2-Amino-5-ethyl-pyrimidine-4,6-diol

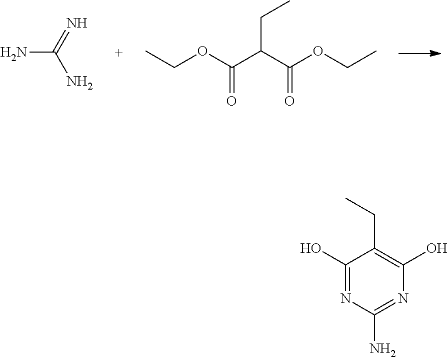

To a 2.0 L three neck flask was added ethanol (800 mL) and solid chunks of sodium metal (23 g, 1.000 mol) were added gradually and carefully under nitrogen flow and the mixture was stirred until completely dissolved. Once cooled back to room temperature guanidine (hydrochloride salt) (36.5 g, 382.1 mmol) and diethyl 2-ethylpropanedioate (56.22 g, 56 mL, 298.7 mmol) were successively added and the reaction was stirred with a mantle set at 80° C. (probe in the solution) for 17 hours. Once cooled to room temperature, the crude mixture was concentrated under reduced pressure to remove most of the ethanol. Water (400 mL) was added, the resulting solution was cooled in an ice bath and acidified to pH of 1-2 using concentrated HCl. The solids were filtered and washed with water (2×100 mL) followed by cold acetone (250 mL). The solid was dried in the fume hood overnight and transferred to a crystallization dish under high vacuum for three days to provide 2-amino-5-ethyl-pyrimidine-4,6-diol (H₂O)₂ (54.8 g, 96%) as a white solid. ESI-MS m/z calc. 155.0695, found 156.2 (M+1)⁺; Retention time: 0.369 minutes. ¹H NMR (300 MHz, DMSO-d₆) ppm 0.87 (t, J=7.2 Hz, 3H), 2.03-2.23 (m, 2H), 6.28 (s, 2H), 10.02-10.45 (m, 2H).

### Step 6: N′-(4,6-dichloro-5-ethyl-pyrimidin-2-yl)-N,N-dimethyl-formamidine

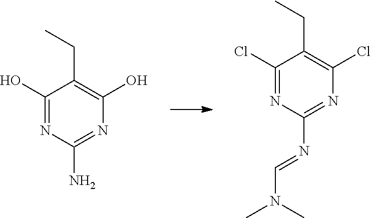

Oxalyl chloride (290.0 g, 200 mL, 2.285 mol) was slowly added to a solution of dimethylformamide (166.4 g, 177 mL, 2.277 mol) in chloroform (1.6 L) and the solution was stirred at room temperature for 30 minutes. 2-Amino-5-ethyl-pyrimidine-4,6-diol (H₂O)₂ (43.55 g, 227.8 mmol) was added then the reaction mixture was heated at 60° C. overnight. Once cooled to room temperature, the reaction mixture was diluted with saturated sodium bicarbonate solution (2.0 L) and stirred vigorously for 15 minutes. A 25% sodium hydroxide solution (250 mL) was added to reach pH ˜8-9. Layers were separated and aqueous layer was extracted with dichloromethane (2×700 mL). Organic layers were combined, dried over sodium sulfate and concentrated under reduced pressure to afford N′-(4,6-dichloro-5-ethyl-pyrimidin-2-yl)-N,N-dimethyl-formamidine (192 g, 341%) as brown oil. The crude material was used for the next step without any further purification. ESI-MS m/z calc. 246.0439, found 247.1 (M+1)⁺; Retention time: 1.25 minutes.

### Step 7: 4,6-Dichloro-5-ethyl-pyrimidin-2-amine

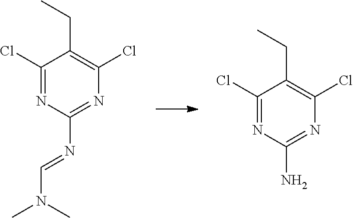

Concentrated hydrochloric acid (117 mL of 12 M, 1.404 mol) was added to a solution of N′-(4,6-dichloro-5-ethyl-pyrimidin-2-yl)-N,N-dimethyl-formamidine (57.9 g, 234.3 mmol) and the mixture was stirred at 50° C. for 150 minutes. The mixture was cooled in the freezer overnight then the solid was filtered, rinsed with cold isopropanol (350 mL) and dried to afford 4,6-dichloro-5-ethyl-pyrimidin-2-amine (35.4 g, 77%) as an off-white solid. ESI-MS m/z calc. 191.0017, found 192.1 (M+1)⁺; Retention time: 2.39 minutes. ¹H NMR (300 MHz, CDCl₃) ppm 1.15 (t, J=7.5 Hz, 3H), 2.75 (q, J=7.3 Hz, 2H), 5.30 (br. s., 2H).

### Step 8: 2-Chloro-3-(4-methylpiperazin-1-yl)phenol

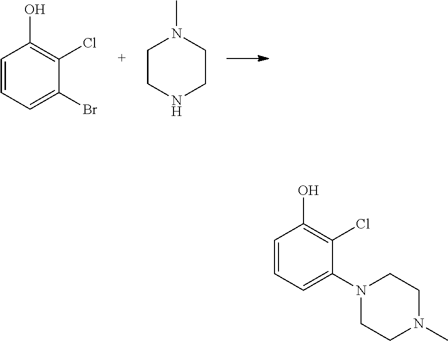

A heterogeneous mixture of 3-bromo-2-chloro-phenol (4.20 g, 20.25 mmol), 1-methylpiperazine (21.5 g, 214.7 mmol), chloro(2-di-t-butylphosphino-2′, 4′, 6′-tri-i-propyl-1,1′-biphenyl)[2-(2-aminoethyl)phenyl] palladium(II) [t-BuXPhos Palladacycle Gen. 1] (2.1 g, 3.058 mmol), and potassium tert-butoxide (4.8 g, 42.78 mmol) in dioxane (120 mL) was sonicated for 15 minutes and heated in a sealed vessel at 50° C. for 16 hours. The reaction mixture was acidified using acetic acid (3.4 mL, 59.79 mmol) then partitioned between DCM (100 mL) and water (100 mL). The organic layer was separated, and the aqueous layer was further extracted with DCM (4×). The combined organic layers were washed once with brine, dried using magnesium sulfate, filtered, and concentrated in vacuo. The crude residue was purified by flash column chromatography on silica gel (gradient: 1 to 10% methanol in dichloromethane) to afford 2-chloro-3-(4-methylpiperazin-1-yl)phenol (3.86 g, 84%) as a yellow solid. ESI-MS m z calc. 226.0873, found 227.12 (M+1)⁺; Retention time: 0.24 minutes (LC method D).

### Step 9: 1-Benzyl-N-(4,6-dichloro-5-ethyl-pyrimidin-2-yl)pyrazole-4-sulfonamide

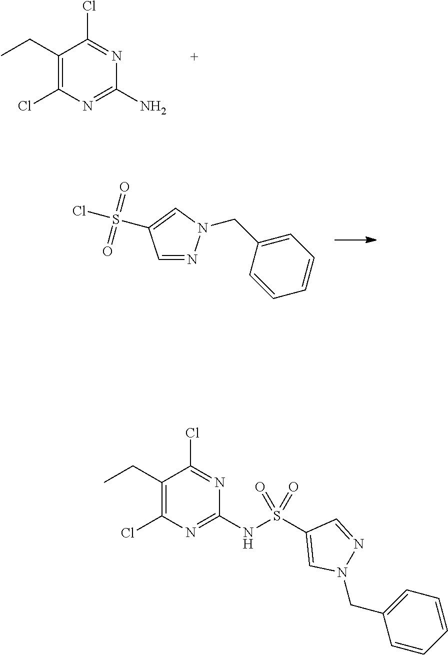 [SMILES: CCc1c(Cl)nc(N)nc1Cl ; O=S(=O)(Cl)c1cnn(Cc2ccccc2)c1 ; CCc1c(Cl)nc(NS(=O)(=O)c2cnn(Cc3ccccc3)c2)nc1Cl]

To a solution of 4,6-dichloro-5-ethyl-pyrimidin-2-amine (628 mg, 3.270 mmol) in DMF (10 mL) at 0° C. was added sodium hydride (530 mg of 60% w/w, 13.25 mmol). The reaction was allowed to warm to 23° C. over 15 minutes. The solution was cooled to 0° C. and 1-benzylpyrazole-4-sulfonyl chloride (998 mg, 3.888 mmol) was added. The reaction mixture was allowed to warm to 23° C. over 15 minutes. The solution was cooled to 0° C., acidified with acetic acid (2.8 mL, 49.24 mmol), and partitioned between ethyl acetate and water. The organic layer was separated, and the aqueous layer was further extracted with ethyl acetate (2×). The combined organics were washed with a saturated sodium bicarbonate solution, brine, dried over magnesium sulfate, filtered, and concentrated to give 1-benzyl-N-(4,6-dichloro-5-ethyl-pyrimidin-2-yl)pyrazole-4-sulfonamide (1.29 g, 96%) ESI-MS m/z calc. 411.03235, found 412.1 (M+1)⁺; Retention time: 0.65 minutes (LC method D).

### Step 10: 1-Benzyl-N-[4-chloro-6-[2-chloro-3-(4-methylpiperazin-1-yl)phenoxy]-5-ethyl-pyrimidin-2-yl]pyrazole-4-sulfonamide

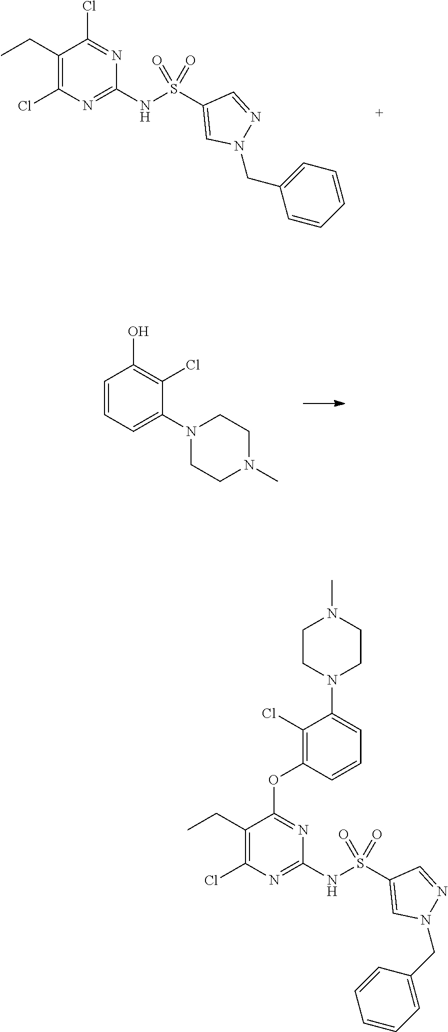 [SMILES: CCc1c(Cl)nc(NS(=O)(=O)c2cnn(Cc3ccccc3)c2)nc1Cl ; CN1CCN(c2cccc(O)c2Cl)CC1 ; CCc1c(Cl)nc(NS(=O)(=O)c2cnn(Cc3ccccc3)c2)nc1Oc1cccc(N2CCN(C)CC2)c1Cl]

A heterogeneous solution of 1-benzyl-N-(4,6-dichloro-5-ethyl-pyrimidin-2-yl)pyrazole-4-sulfonamide (1.29 g, 3.129 mmol), 2-chloro-3-(4-methylpiperazin-1-yl)phenol (701 mg, 3.092 mmol), and potassium carbonate (approximately 1.298 g, 9.390 mmol) in NMP (3.858 mL) was heated in a sealed vial to 120° C. for 6 hours. The reaction was diluted with water (10 mL) and acetic acid (1.1 mL, 19.34 mmol) was added. The mixture was extracted with dichloromethane (3×20 mL). The organics were combined, washed with brine, dried over magnesium sulfate and evaporated. The crude material was purified by silica gel chromatography eluting with 0-10% methanol in dichloromethane to give 1-benzyl-N-[4-chloro-6-[2-chloro-3-(4-methylpiperazin-1-yl)phenoxy]-5-ethyl-pyrimidin-2-yl]pyrazole-4-sulfonamide (923 mg, 49%) ESI-MS m/z calc. 601.14294, found 602.2 (M+1)⁺; Retention time: 0.59 minutes (LC method D).

### Step 11: 1-Benzyl-N-[4-[2-chloro-3-(4-methylpiperazin-1-yl)phenoxy]-5-ethyl-6-(o-tolyl)pyrimidin-2-yl]pyrazole-4-sulfonamide

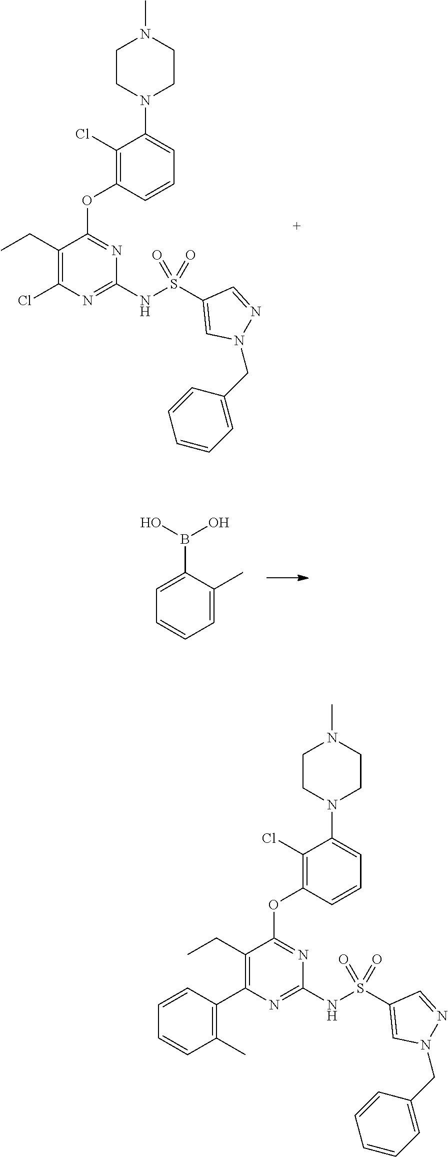

1-Benzyl-N-[4-chloro-6-[2-chloro-3-(4-methylpiperazin-1-yl)phenoxy]-5-ethyl-pyrimidin-2-yl]pyrazole-4-sulfonamide (124 mg, 0.2058 mmol), o-tolylboronic acid (34 mg, 0.2501 mmol), tetrakis(triphenylphosphine)palladium (0) (24 mg, 0.02077 mmol), and aqueous potassium carbonate (400 μL of 2 M, 0.8000 mmol) were combined in dioxane (2 mL) and heated at 120° C. in a sealed vial for 3 hours. The reaction was cooled and filtered. The crude material was purified by LC/MS utilizing a gradient of 1-99% acetonitrile in 5 mM aqueous HCl to yield 1-benzyl-N-[4-[2-chloro-3-(4-methylpiperazin-1-yl)phenoxy]-5-ethyl-6-(o-tolyl)pyrimidin-2-yl]pyrazole-4-sulfonamide (hydrochloride salt) (75.6 mg, 53%). ESI-MS m z calc. 657.2289, found 658.4 (M+1)⁺; Retention time: 0.62 minutes (LC method A).

### Step 12: N-[4-[2-chloro-3-(4-methylpiperazin-1-yl)phenoxy]-5-ethyl-6-(o-tolyl)pyrimidin-2-yl]-1H-pyrazole-4-sulfonamide

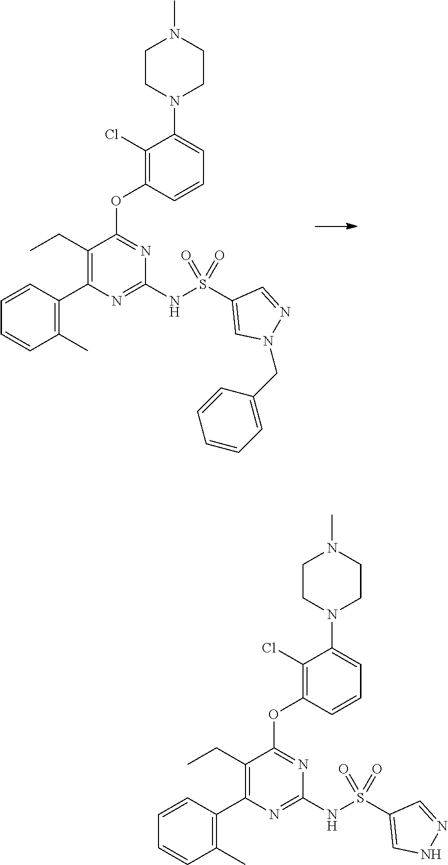 [SMILES: CCc1c(Oc2cccc(N3CCN(C)CC3)c2Cl)nc(NS(=O)(=O)c2cnn(Cc3ccccc3)c2)nc1-c1ccccc1C ; CCc1c(Oc2cccc(N3CCN(C)CC3)c2Cl)nc(NS(=O)(=O)c2cn[nH]c2)nc1-c1ccccc1C]

1-Benzyl-N-[4-[2-chloro-3-(4-methylpiperazin-1-yl)phenoxy]-5-ethyl-6-(o-tolyl)pyrimidin-2-yl]pyrazole-4-sulfonamide (hydrochloride salt) (75.6 mg, 0.1088 mmol), dihydroxypalladium (19 mg of 10% w/w, 0.01353 mmol), and HCl (60 μL of 12 M, 0.7200 mmol) were combined in ethanol (3 mL) under a balloon of hydrogen at 70° C. The reaction was stirred for 15 minutes, then filtered and evaporated. The crude material was purified by LC/MS utilizing a gradient of 1-99% acetonitrile in 5 mM aqueous HCl to yield N-[4-[2-chloro-3-(4-methylpiperazin-1-yl)phenoxy]-5-ethyl-6-(o-tolyl)pyrimidin-2-yl]-1H-pyrazole-4-sulfonamide (hydrochloride salt) (30 mg, 45%). ESI-MS m/z calc. 567.18195, found 568.3 (M+1)⁺; Retention time: 1.31 minutes (LC method A). ¹H NMR (400 MHz, DMSO-d₆) δ 10.58 (s, 1H), 7.51 (t, J=8.1 Hz, 1H), 7.43-7.17 (m, 8H), 3.54 (d, J=19.9 Hz, 4H), 3.19 (dt, J=24.1, 11.2 Hz, 4H), 2.86 (d, J=4.7 Hz, 3H), 2.08 (s, 3H), 1.03 (t, J=7.4 Hz, 3H). One CH₂ hidden under DMSO peak.

### Example 272: Preparation of Compound 914

### Step 1: 1-Benzyl-N-[4-[2-chloro-3-(4-methylpiperazin-1-yl)phenoxy]-5-ethyl-6-(2-isobutoxyphenyl)pyrimidin-2-yl]pyrazole-4-sulfonamide

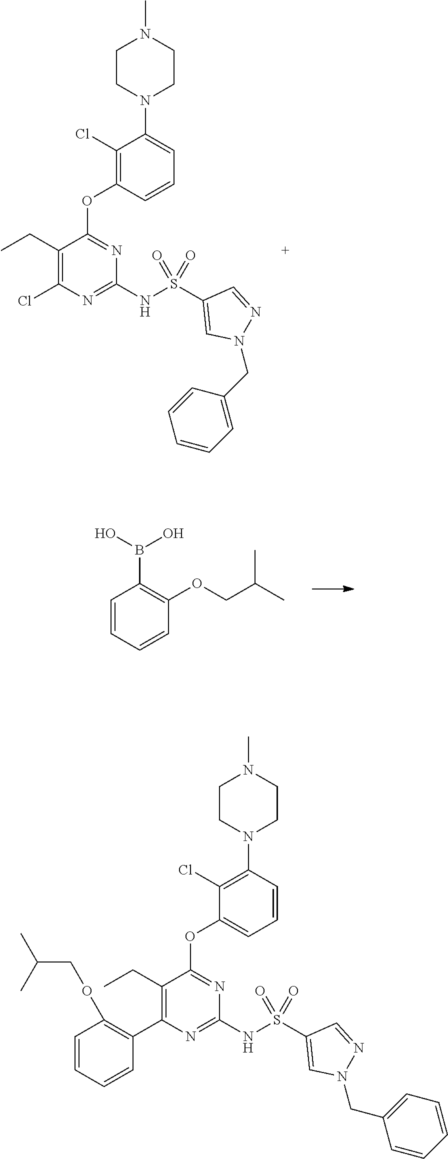 [SMILES: CCc1c(Cl)nc(NS(=O)(=O)c2cnn(Cc3ccccc3)c2)nc1Oc1cccc(N2CCN(C)CC2)c1Cl ; CC(C)COc1ccccc1B(O)O ; CCc1c(Oc2cccc(N3CCN(C)CC3)c2Cl)nc(NS(=O)(=O)c2cnn(Cc3ccccc3)c2)nc1-c1ccccc1OCC(C)C]

1-benzyl-N-[4-chloro-6-[2-chloro-3-(4-methylpiperazin-1-yl)phenoxy]-5-ethyl-pyrimidin-2-yl]pyrazole-4-sulfonamide (50 mg, 0.08298 mmol), (2-isobutoxyphenyl)boronic acid (approximately 19.32 mg, 0.09958 mmol), tetrakis(triphenylphosphine)palladium (0) (approximately 9.589 mg, 0.008298 mmol), and 2 M aqueous potassium carbonate (approximately 166.0 μL of 2 M, 0.3319 mmol) were combined in dioxane (0.8 mL) and irradiated in the microwave for 30 minutes at 120° C. The reaction mixture was filtered and purified by LC/MS utilizing a gradient of 1-99% acetonitrile in 5 mM aqueous HCl to yield 1-benzyl-N-[4-[2-chloro-3-(4-methylpiperazin-1-yl)phenoxy]-5-ethyl-6-(2-isobutoxyphenyl)pyrimidin-2-yl]pyrazole-4-sulfonamide (hydrochloride salt) (25 mg, 40%). ESI-MS m/z calc. 715.27075, found 716.4 (M+1)⁺; Retention time: 1.58 minutes (LC method A).

### Step 2: N-[4-[2-chloro-3-(4-methylpiperazin-1-yl)phenoxy]-5-ethyl-6-(2-isobutoxyphenyl)pyrimidin-2-yl]-1H-pyrazole-4-sulfonamide

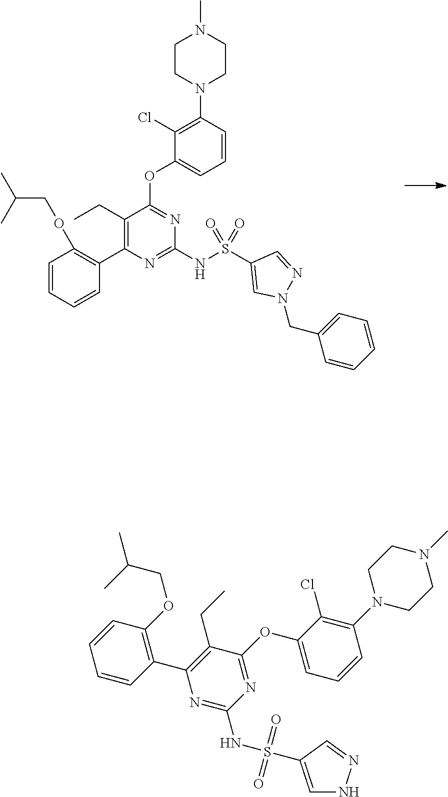 [SMILES: CCc1c(Oc2cccc(N3CCN(C)CC3)c2Cl)nc(NS(=O)(=O)c2cnn(Cc3ccccc3)c2)nc1-c1ccccc1OCC(C)C ; CCc1c(Oc2cccc(N3CCN(C)CC3)c2Cl)nc(NS(=O)(=O)c2cn[nH]c2)nc1-c1ccccc1OCC(C)C]

To a mixture of 1-benzyl-N-[4-[2-chloro-3-(4-methylpiperazin-1-yl)phenoxy]-5-ethyl-6-(2-isobutoxyphenyl)pyrimidin-2-yl]pyrazole-4-sulfonamide (hydrochloride salt) (25 mg, 0.03321 mmol) and palladium hydroxide (approximately 99.99 mg of 20% w/w, 0.1424 mmol) in ethanol (2 mL) was added 5 drops of concentrated HCl (approximately 2.768 μL of 12 M, 0.03321 mmol) and the reaction was heated at 70° C. for 15 minutes under an atmosphere of hydrogen. The reaction was filtered and evaporated to dryness. The crude material was purified by LC/MS utilizing a gradient of 1-99% acetonitrile in 5 mM aqueous HCl to yield N-[4-[2-chloro-3-(4-methylpiperazin-1-yl)phenoxy]-5-ethyl-6-(2-isobutoxyphenyl)pyrimidin-2-yl]-1H-pyrazole-4-sulfonamide (hydrochloride salt) (8.1 mg, 36%). ESI-MS m/z calc. 625.2238, found 626.3 (M+1)⁺; Retention time: 1.45 minutes (LC method A).

### Example 273: Preparation of Compound 915

### Step 1: 1-Benzyl-N-[4-[2-chloro-3-(4-methylpiperazin-1-yl)phenoxy]-6-(2-ethoxyphenyl)-5-ethyl-pyrimidin-2-yl]pyrazole-4-sulfonamide

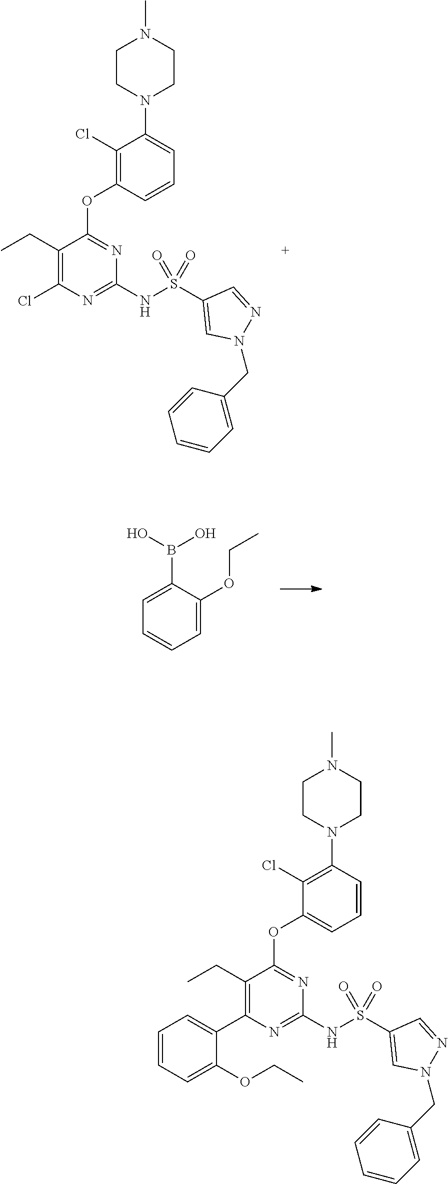 [SMILES: CCc1c(Cl)nc(NS(=O)(=O)c2cnn(Cc3ccccc3)c2)nc1Oc1cccc(N2CCN(C)CC2)c1Cl ; CCOc1ccccc1B(O)O ; CCOc1ccccc1-c1nc(NS(=O)(=O)c2cnn(Cc3ccccc3)c2)nc(Oc2cccc(N3CCN(C)CC3)c2Cl)c1CC]

1-Benzyl-N-[4-chloro-6-[2-chloro-3-(4-methylpiperazin-1-yl)phenoxy]-5-ethyl-pyrimidin-2-yl]pyrazole-4-sulfonamide (50 mg, 0.08298 mmol), (2-ethoxyphenyl)boronic acid (approximately 16.53 mg, 0.09958 mmol), tetrakis(triphenylphosphine)palladium (0) (approximately 9.589 mg, 0.008298 mmol), and 2 M aqueous potassium carbonate (approximately 166.0 μL of 2 M, 0.3319 mmol) were combined in dioxane (0.8 mL) and irradiated in the microwave for 30 minutes at 120° C. The reaction mixture was filtered and purified by LC/MS utilizing a gradient of 1-99% acetonitrile in 5 mM aqueous HCl to yield 1-benzyl-N-[4-[2-chloro-3-(4-methylpiperazin-1-yl)phenoxy]-6-(2-ethoxyphenyl)-5-ethyl-pyrimidin-2-yl]pyrazole-4-sulfonamide (hydrochloride salt) (35.8 mg, 60%). ESI-MS m/z calc. 687.23944, found 688.4 (M+1)⁺; Retention time: 0.6 minutes (LC method D).

### Step 2: N-[4-[2-chloro-3-(4-methylpiperazin-1-yl)phenoxy]-6-(2-ethoxyphenyl)-5-ethyl-pyrimidin-2-yl]-1H-pyrazole-4-sulfonamide

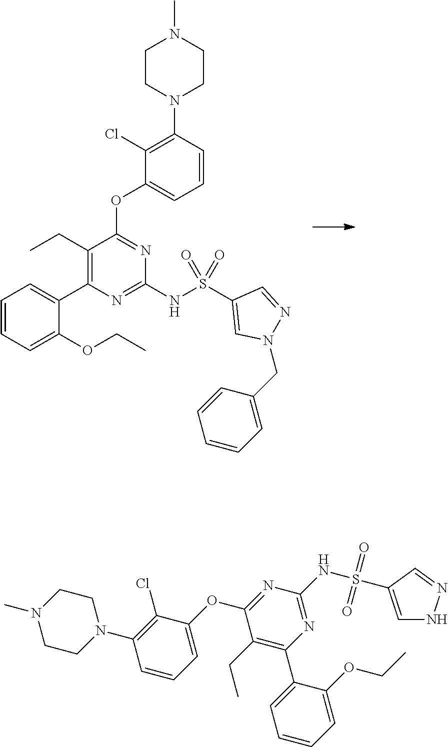 [SMILES: CCOc1ccccc1-c1nc(NS(=O)(=O)c2cnn(Cc3ccccc3)c2)nc(Oc2cccc(N3CCN(C)CC3)c2Cl)c1CC ; CCOc1ccccc1-c1nc(NS(=O)(=O)c2cn[nH]c2)nc(Oc2cccc(N3CCN(C)CC3)c2Cl)c1CC]

To a mixture of 1-benzyl-N-[4-[2-chloro-3-(4-methylpiperazin-1-yl)phenoxy]-6-(2-ethoxyphenyl)-5-ethyl-pyrimidin-2-yl]pyrazole-4-sulfonamide (hydrochloride salt) (28.3 mg, 0.03905 mmol) and palladium hydroxide (approximately 99.99 mg of 20% w/w, 0.1424 mmol) in ethanol (2 mL) was added 5 drops of concentrated HCl (approximately 3.254 μL of 12 M, 0.03905 mmol) and the reaction was heated at 70° C. for 15 minutes under an atmosphere of hydrogen. The reaction was filtered and evaporated to dryness. The crude material was purified by LC/MS utilizing a gradient of 1-99% acetonitrile in 5 mM aqueous HCl to yield N-[4-[2-chloro-3-(4-methylpiperazin-1-yl)phenoxy]-6-(2-ethoxyphenyl)-5-ethyl-pyrimidin-2-yl]-H-pyrazole-4-sulfonamide (hydrochloride salt) (9.6 mg, 38%). ESI-MS m/z calc. 597.1925, found 598.5 (M+1)⁺; Retention time: 1.21 minutes (LC method A).

### Example 274: Preparation of Compound 916

### Step 1: 1-Benzyl-N-[4-[2-chloro-3-(4-methylpiperazin-1-yl)phenoxy]-5-ethyl-6-(2-isopropoxyphenyl)pyrimidin-2-yl]pyrazole-4-sulfonamide

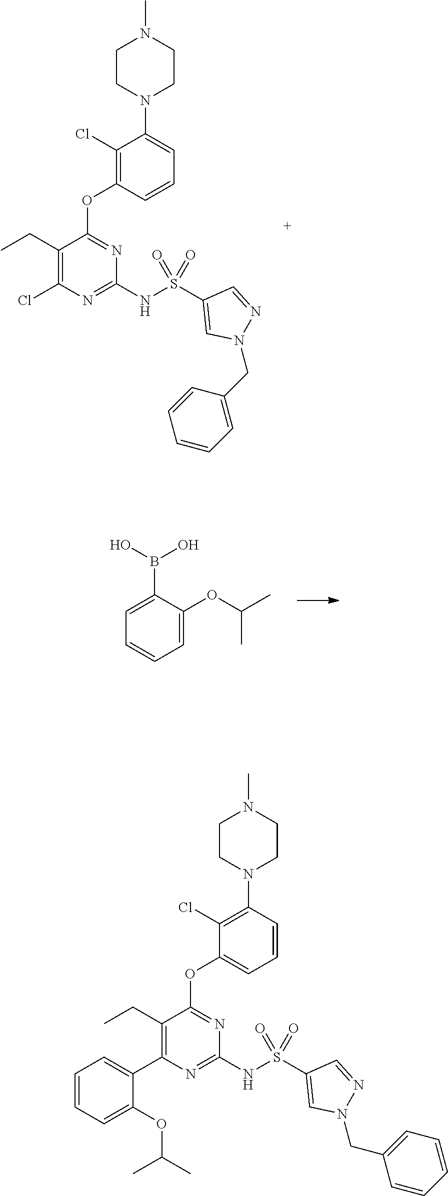 [SMILES: CCc1c(Cl)nc(NS(=O)(=O)c2cnn(Cc3ccccc3)c2)nc1Oc1cccc(N2CCN(C)CC2)c1Cl ; CC(C)Oc1ccccc1B(O)O ; CCc1c(Oc2cccc(N3CCN(C)CC3)c2Cl)nc(NS(=O)(=O)c2cnn(Cc3ccccc3)c2)nc1-c1ccccc1OC(C)C]

1-Benzyl-N-[4-chloro-6-[2-chloro-3-(4-methylpiperazin-1-yl)phenoxy]-5-ethyl-pyrimidin-2-yl]pyrazole-4-sulfonamide (50 mg, 0.08298 mmol), (2-isopropoxyphenyl)boronic acid (approximately 17.93 mg, 16.68 μL, 0.09958 mmol), tetrakis(triphenylphosphine)palladium (0) (approximately 9.589 mg, 0.008298 mmol), and 2 M aqueous potassium carbonate (approximately 166.0 μL of 2 M, 0.3319 mmol) were combined in dioxane (0.8 mL) and irradiated in the microwave for 30 minutes at 120° C. The reaction mixture was filtered and purified by LC/MS utilizing a gradient of 1-99% acetonitrile in 5 mM aqueous HCl to yield 1-benzyl-N-[4-[2-chloro-3-(4-methylpiperazin-1-yl)phenoxy]-5-ethyl-6-(2-isopropoxyphenyl)pyrimidin-2-yl]pyrazole-4-sulfonamide (hydrochloride salt) (21.6 mg, 35%). ESI-MS m/z calc. 701.2551, found 702.4 (M+1)⁺; Retention time: 0.61 minutes; LC method D.

### Step 2: N-[4-[2-chloro-3-(4-methylpiperazin-1-yl)phenoxy]-5-ethyl-6-(2-isopropoxyphenyl)pyrimidin-2-yl]-1H-pyrazole-4-sulfonamide

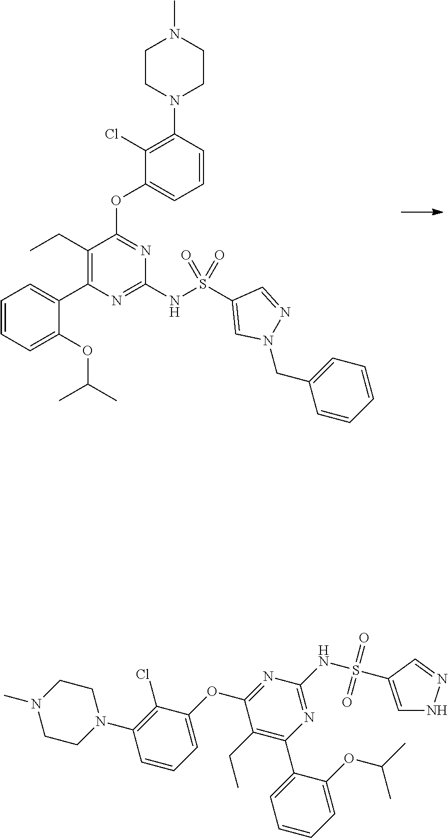 [SMILES: CCc1c(Oc2cccc(N3CCN(C)CC3)c2Cl)nc(NS(=O)(=O)c2cnn(Cc3ccccc3)c2)nc1-c1ccccc1OC(C)C ; CCc1c(Oc2cccc(N3CCN(C)CC3)c2Cl)nc(NS(=O)(=O)c2cn[nH]c2)nc1-c1ccccc1OC(C)C]

To a mixture of 1-benzyl-N-[4-[2-chloro-3-(4-methylpiperazin-1-yl)phenoxy]-5-ethyl-6-(2-isopropoxyphenyl)pyrimidin-2-yl]pyrazole-4-sulfonamide (hydrochloride salt) (23 mg, 0.03113 mmol) and palladium hydroxide (approximately 99.99 mg of 20% w/w, 0.1424 mmol) in ethanol (2 mL) was added 5 drops of concentrated HCl (approximately 2.594 μL of 12 M, 0.03113 mmol) and the reaction was heated at 70° C. for 15 minutes under an atmosphere of hydrogen. The reaction was filtered and evaporated to dryness. The crude material was purified by LC/MS utilizing a gradient of 1-99% acetonitrile in 5 mM aqueous HCl to yield N-[4-[2-chloro-3-(4-methylpiperazin-1-yl)phenoxy]-5-ethyl-6-(2-isopropoxyphenyl)pyrimidin-2-yl]-1H-pyrazole-4-sulfonamide (hydrochloride salt) (9.1 mg, 44%). ESI-MS m/z calc. 611.2081, found 612.3 (M+1)⁺; Retention time: 1.24 minutes (LC method A).

### Example 275: Preparation of Compound 917

### Step 1: 1-Benzyl-N-[4-[2-chloro-3-(4-methylpiperazin-1-yl)phenoxy]-5-ethyl-6-phenyl-pyrimidin-2-yl]pyrazole-4-sulfonamide

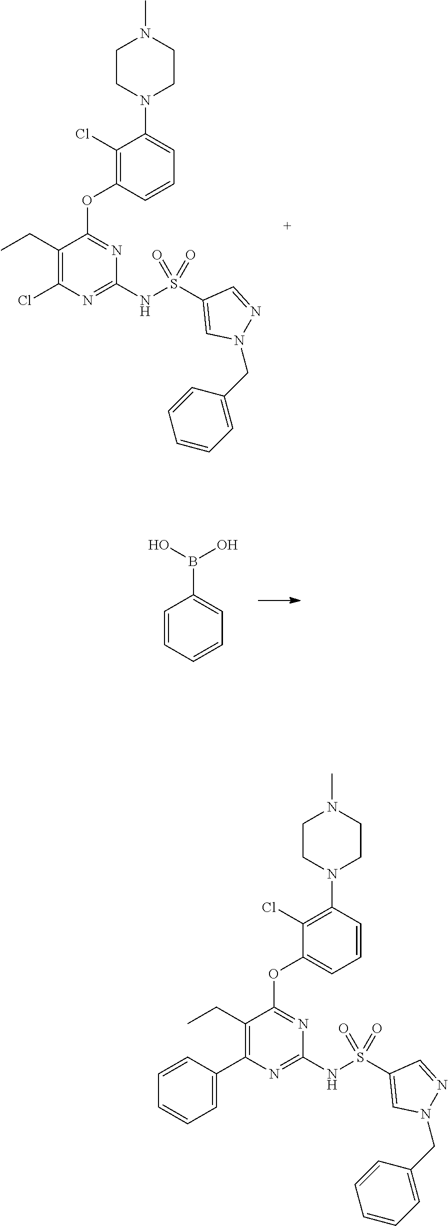 [SMILES: CCc1c(Cl)nc(NS(=O)(=O)c2cnn(Cc3ccccc3)c2)nc1Oc1cccc(N2CCN(C)CC2)c1Cl ; OB(O)c1ccccc1 ; CCc1c(Oc2cccc(N3CCN(C)CC3)c2Cl)nc(NS(=O)(=O)c2cnn(Cc3ccccc3)c2)nc1-c1ccccc1]

1-Benzyl-N-[4-chloro-6-[2-chloro-3-(4-methylpiperazin-1-yl)phenoxy]-5-ethyl-pyrimidin-2-yl]pyrazole-4-sulfonamide (50 mg, 0.08298 mmol), phenylboronic acid (approximately 12.14 mg, 0.09958 mmol) tetrakis(triphenylphosphine)palladium (0) (approximately 9.589 mg, 0.008298 mmol), and 2M aqueous potassium carbonate (approximately 166.0 μL of 2 M, 0.3319 mmol) were combined in dioxane (0.8 mL) and irradiated in the microwave for 30 minutes at 120° C. The reaction mixture was filtered and purified by LC/MS utilizing a gradient of 1-99% acetonitrile in 5 mM aqueous HCl to yield 1-benzyl-N-[4-[2-chloro-3-(4-methylpiperazin-1-yl)phenoxy]-5-ethyl-6-phenyl-pyrimidin-2-yl]pyrazole-4-sulfonamide (hydrochloride salt) (36.7 mg, 65%). ESI-MS m/z calc. 643.21326, found 644.3 (M+1)⁺; Retention time: 0.57 minutes (LC method D).

### Step 2: N-[4-[2-chloro-3-(4-methylpiperazin-1-yl)phenoxy]-5-ethyl-6-phenyl-pyrimidin-2-yl]-1H-pyrazole-4-sulfonamide

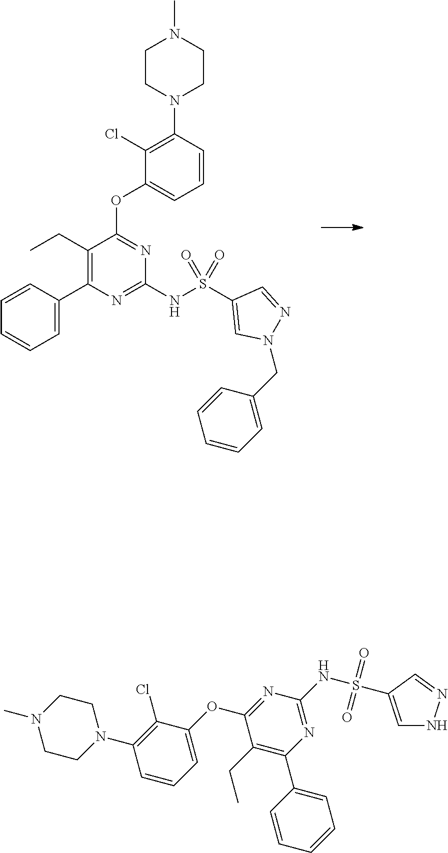 [SMILES: CCc1c(Oc2cccc(N3CCN(C)CC3)c2Cl)nc(NS(=O)(=O)c2cnn(Cc3ccccc3)c2)nc1-c1ccccc1 ; CCc1c(Oc2cccc(N3CCN(C)CC3)c2Cl)nc(NS(=O)(=O)c2cn[nH]c2)nc1-c1ccccc1]

To a mixture of 1-benzyl-N-[4-[2-chloro-3-(4-methylpiperazin-1-yl)phenoxy]-5-ethyl-6-phenyl-pyrimidin-2-yl]pyrazole-4-sulfonamide (hydrochloride salt) (27.8 mg, 0.04084 mmol) and palladium hydroxide (approximately 99.99 mg of 20% w/w, 0.1424 mmol) in ethanol (2 mL) was added 5 drops of concentrated HCl (approximately 3.403 μL of 12 M, 0.04084 mmol) and the reaction was heated at 70° C. for 15 minutes under an atmosphere of hydrogen. The reaction was filtered and evaporated to dryness. The crude material was purified by LC/MS utilizing a gradient of 1-99% acetonitrile in 5 mM aqueous HCl to yield N-[4-[2-chloro-3-(4-methylpiperazin-1-yl)phenoxy]-5-ethyl-6-phenyl-pyrimidin-2-yl]-1H-pyrazole-4-sulfonamide (hydrochloride salt) (3.2 mg, 13%). ESI-MS m/z calc. 553.16626, found 554.2 (M+1)⁺; Retention time: 1.15 minutes (LC method A).

### Example 276: Preparation of Compound 918

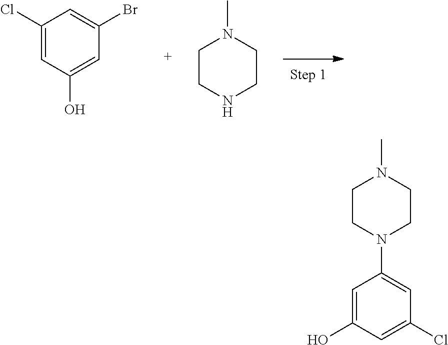

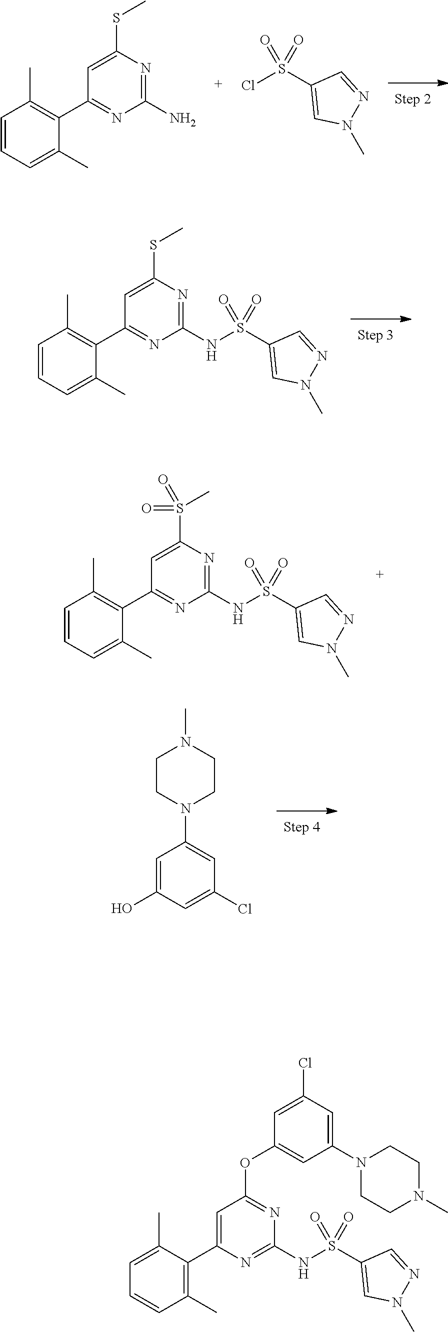

### Step 1: 3 -Cholor-5(4-methlpiperazin-1-yl)phenol

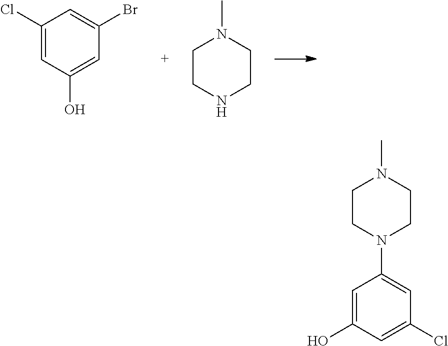

In a glass vial was added 3-bromo-5-chloro-phenol (300 mg, 1.446 mmol), [2-(2-aminoethyl)phenyl]-chloro-palladium;ditert-butyl-[2-(2,4,6-triisopropylphenyl)phenyl]phosphane (XPhos Pd G1)(approximately 99.30 mg, 0.1446 mmol), 1-methylpiperazine (approximately 152.0 mg, 168.5 μL, 1.518 mmol), and dioxane (10 mL) and the mixture is sparged with nitrogen for 30 minutes and then solid sodium tert-butoxide (approximately 277.9 mg, 2.892 mmol) was added. The reaction was stirred under nitrogen pressure for 15 minutes at room temperature and then poured into a saturated aqueous solution of ammonium chloride (25 mL) and dichloromethane (25 mL). The layers were separated, and the aqueous layer was extracted with dichloromethane (25 mL). The organic layers were and dried over anhydrous magnesium sulfate, filtered and concentrated in vacuo. The residue was washed with ethyl acetate (2×10 mL) to remove residual dioxane and DCM and then taken up in ethyl acetate (3.5 mL, 10 volumes) and heated to 80° C. to dissolve everything and then stirred and cooled to room temperature over 1 hour and stirred at room temperature for 16 hours. The solid was filtered and washed with ethyl acetate (0.5 mL) followed by diethyl ether (1 mL) to afford the title compound 3-chloro-5-(4-methylpiperazin-1-yl)phenol. ESI-MS m/z calc. 226.0873, found 227.29 (M+1)⁺; Retention time: 0.68 minutes; LC method A.

### Step 2: N-[4-(2,6-Dimethylphenyl)-6-methylsulfanyl-pyrimidin-2-yl]-1-methyl-pyrazole-4-sulfonamide

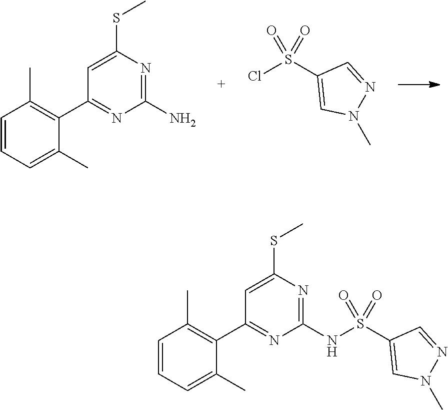

To a 250 mL round-bottomed flask equipped with a magnetic stir bar were added 4-(2,6-dimethylphenyl)-6-methylsulfanyl-pyrimidin-2-amine (3.8460 g, 15.68 mmol) and dimethylformamide (50.0 mL), and this mixture was cooled to 0° C. 60% NaH (2.30 g, 57.51 mmol) was added in one portion, and the reaction mixture was warmed to room temperature over 30 minutes. The mixture was cooled to 0° C., upon which 1-methylpyrazole-4-sulfonyl chloride (3.8460 g, 21.29 mmol) was added in one portion. This solution was stirred at room temperature for 1 hours, then quenched by a slow transfer onto ice-cold 1 N HCl (75 mL). The mixture was extracted with ethyl acetate (3×100 mL). The combined organic extracts were washed with water (200 mL) and saturated aqueous sodium chloride solution (100 mL), then dried over sodium sulfate, filtered, and evaporated in vacuo. This crude product was purified by silica gel chromatography (80 g of silica, 0 to 50% gradient of ethyl acetate/hexanes) to give three products: recovered starting material (0.297 g, 7.7%), N-[4-(2,6-dimethylphenyl)-6-methylsulfanyl-pyrimidin-2-yl]-1-methyl-pyrazole-4-sulfonamide (2.451 g, 34%) ESI-MS m z calc. 389.09802, found 390.1 (M+1)⁺; Retention time: 0.56 minutes and 5-chloro-N-[4-(2,6-dimethylphenyl)-6-methylsulfanyl-pyrimidin-2-yl]-1-methyl-pyrazole-4-sulfonamide (0.8505 g, 13%) ESI-MS m/z calc. 423.05905, found 424.1 (M+1)⁺; Retention time: 0.59 minutes (LC method D).

### Step 3: N-[4-(2,6-Dimethylphenyl)-6-methylsulfonyl-pyrimidin-2-yl]-1-methyl-pyrazole-4-sulfonamide

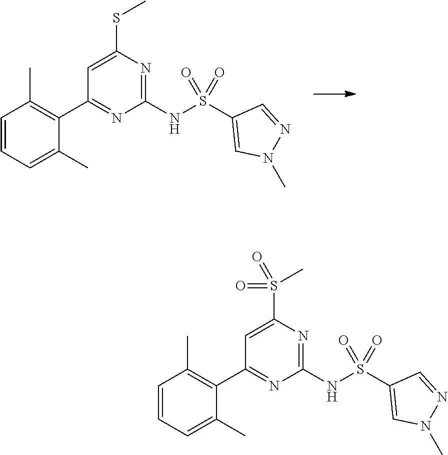 [SMILES: CSc1cc(-c2c(C)cccc2C)nc(NS(=O)(=O)c2cnn(C)c2)n1 ; Cc1cccc(C)c1-c1cc(S(C)(=O)=O)nc(NS(=O)(=O)c2cnn(C)c2)n1]

To a 100 mL round-bottomed flask equipped with a magnetic stir bar, N-[4-(2,6-dimethylphenyl)-6-methylsulfanyl-pyrimidin-2-yl]-1-methyl-pyrazole-4-sulfonamide (2.451 g, 5.789 mmol) and dichloromethane (55 mL) were added, followed by 77% m-chloroperbenzoic acid (2.921 g, 13.03 mmol). This solution was stirred at room temperature for 1.5 hours. The reaction mixture was quenched with solid sodium thiosulfate (5.020 g, 31.75 mmol). This mixture was stirred for another 1.5 hours at room temperature. The reaction mixture was diluted with dichloromethane (50 mL), then washed with water (2×40 mL) and saturated aqueous sodium chloride solution (40 mL). The organic layer was then dried over sodium sulfate, filtered, and evaporated in vacuo. This crude product was first purified by dissolution in dichloromethane (20 mL) and filtering away most of the m-chlorobenzoic acid waste on a Buchner funnel. The remaining solution was then purified by silica gel chromatography (80 g of silica, 0 to 70% gradient of ethyl acetate/hexanes) to give N-[4-(2,6-dimethylphenyl)-6-methylsulfonyl-pyrimidin-2-yl]-1-methyl-pyrazole-4-sulfonamide (2.1205 g, 87%) ESI-MS m z calc. 421.08786, found 422.1 (M+1)⁺; Retention time: 0.52 minutes. (LC method D).

### Step 4: N-[4-[3-chloro-5-(4-methylpiperazin-1-yl)phenoxy]-6-(2,6-dimethylphenyl)pyrimidin-2-yl]-1-methyl-pyrazole-4-sulfonamide

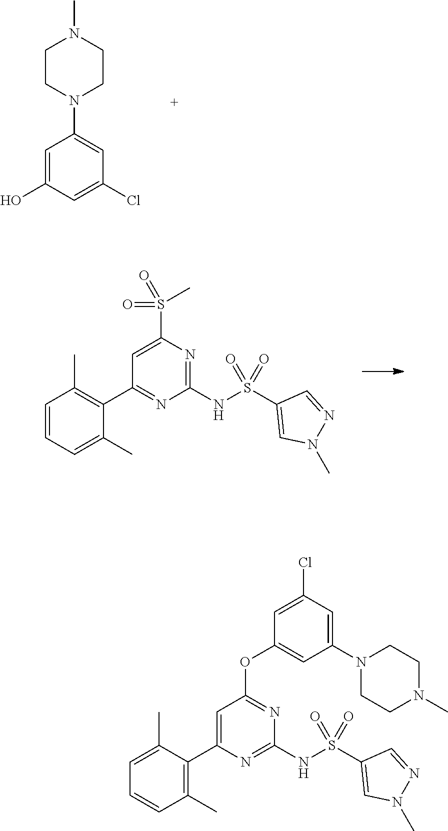 [SMILES: CN1CCN(c2cc(O)cc(Cl)c2)CC1 ; Cc1cccc(C)c1-c1cc(S(C)(=O)=O)nc(NS(=O)(=O)c2cnn(C)c2)n1 ; Cc1cccc(C)c1-c1cc(Oc2cc(Cl)cc(N3CCN(C)CC3)c2)nc(NS(=O)(=O)c2cnn(C)c2)n1]

A mixture of 3-chloro-5-(4-methylpiperazin-1-yl)phenol (approximately 16.14 mg, 0.07118 mmol), Cs₂CO₃ (approximately 46.40 mg, 0.1424 mmol)N-[4-(2,6-dimethylphenyl)-6-methylsulfonyl-pyrimidin-2-yl]-1-methyl-pyrazole-4-sulfonamide (15 mg, 0.03559 mmol) in NMP (0.6 mL) was heated to 110° C. for 2 hours and then cooled to room temperature. The reaction mixture was filtered, and the resulting residue diluted with 0.8 mL MeOH, and purified by reverse phase chromatography using a 15 minute gradient of 20% MeCN in water to 80% MeCN (HCl modifier) to give the title compound. N-[4-[3-chloro-5-(4-methylpiperazin-1-yl)phenoxy]-6-(2,6-dimethylphenyl)pyrimidin-2-yl]-1-methyl-pyrazole-4-sulfonamide (7.9 mg, 33%). ESI-MS m/z calc. 567.18195, found 568.53 (M+1)⁺; Retention time: 1.36 minutes; LC method A.

### Example 277: Preparation of Compound 919

### Step 1: N-[4-[3-bromo-2-(trifluoromethyl)phenoxy]-6-(2,6-dimethylphenyl)pyrimidin-2-yl]-1-methyl-pyrazole-4-sulfonamide

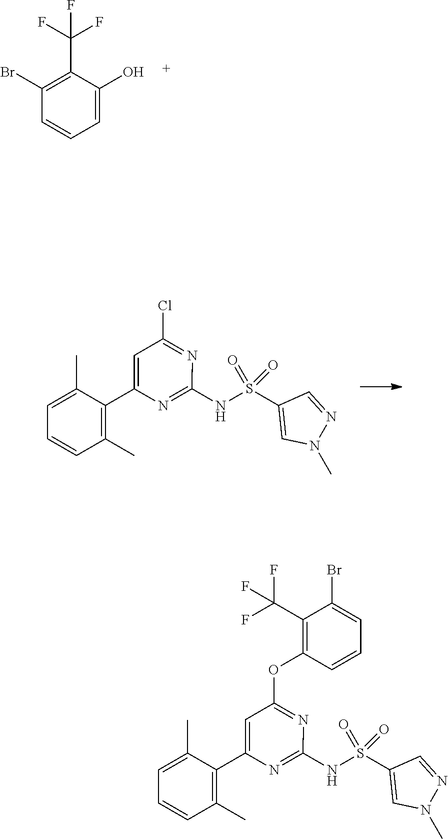 [SMILES: Oc1cccc(Br)c1C(F)(F)F ; Cc1cccc(C)c1-c1cc(Cl)nc(NS(=O)(=O)c2cnn(C)c2)n1 ; Cc1cccc(C)c1-c1cc(Oc2cccc(Br)c2C(F)(F)F)nc(NS(=O)(=O)c2cnn(C)c2)n1]

A mixture of N-[4-chloro-6-(2,6-dimethylphenyl)pyrimidin-2-yl]-1-methyl-pyrazole-4-sulfonamide (50 mg, 0.1323 mmol), 3-bromo-2-(trifluoromethyl)phenol (approximately 83.53 mg, 0.3466 mmol), and Cs₂CO₃ (approximately 188.3 mg, 0.5778 mmol) in NMP (1 mL) was stirred at 110° C. for 16 hours and then cooled to room temperature. The solution was filtered and the filtrate diluted with 0.8 mL MeOH, and purified by reverse phase chromatography using a 15 minute gradient of 1% MeCN in water to 99% MeCN (HCl modifier) to give N-[4-[3-bromo-2-(trifluoromethyl)phenoxy]-6-(2,6-dimethylphenyl)pyrimidin-2-yl]-1-methyl-pyrazole-4-sulfonamide (55.3 mg, 72%) ESI-MS m/z calc. 581.0344, found 582.36 (M+1)⁺; Retention time: 2.03 minutes. (LC method A).

### Step 2: N-[4-(2,6-Dimethylphenyl)-6-[3-(4-methylpiperazin-1-yl)-2-(trifluoromethyl)phenoxy]pyrimidin-2-yl]-1-methyl-pyrazole-4-sulfonamide

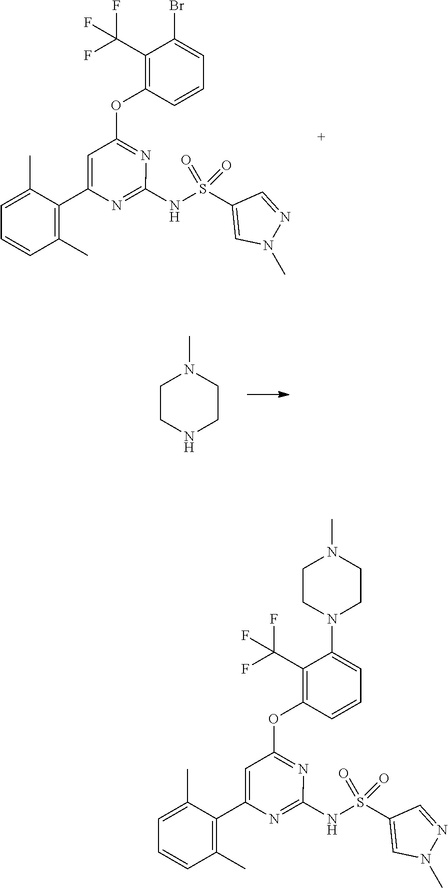

A dioxane (0.6 mL) mixture of N-[4-[3-bromo-2-(trifluoromethyl)phenoxy]-6-(2,6-dimethylphenyl)pyrimidin-2-yl]-1-methyl-pyrazole-4-sulfonamide (15 mg, 0.02576 mmol), 1-methylpiperazine (approximately 31.19 mg, 0.3114 mmol), Chloro(2-di-t-butylphosphino-2′, 4′, 6′-tri-i-propyl-1,1′-biphenyl)[2-(2-aminoethyl)phenyl] palladium(II)[t-BuXPhos Palladacycle Gen. 1] (8.4 mg, 0.01223 mmol), and NaOtBu (12.2 mg, 0.1269 mmol) was sparged with nitrogen for 1 minute and then stirred under a purge of nitrogen for 15 minutes at room temperature. HCl (130 μL of 1 M, 0.1300 mmol) was added to quench the reaction. The solution was filtered and the filtrate dissolved in 0.8 mL MeOH, and purified by reverse phase chromatography using a 15 minute gradient of 20% MeCN in water to 80% MeCN (HCl modifier) to give N-[4-(2,6-dimethylphenyl)-6-[3-(4-methylpiperazin-1-yl)-2-(trifluoromethyl)phenoxy]pyrimidin-2-yl]-1-methyl-pyrazole-4-sulfonamide (hydrochloride salt) (2.4 mg, 14%). ¹H NMR (400 MHz, DMSO-d₆) δ 11.69 (s, 1H), 10.28 (s, 1H), 7.83-7.77 (m, 1H), 7.46 (d, J=8.2 Hz, 1H), 7.32 (d, J=8.2 Hz, 1H), 7.30-7.21 (m, 1H), 7.19-7.01 (m, 3H), 6.70 (s, 1H), 3.77 (s, 3H), 3.56-3.50 (m, 2H), 3.33-3.26 (m, 2H), 3.25-3.12 (m, 4H), 2.87 (d, J=4.6 Hz, 3H), 2.03 (s, 6H). ESI-MS m/z calc. 601.2083, found 602.6 (M+1)⁺; Retention time: 1.43 minutes (LC method A).

### Example 278: Preparation of Compound 920

### Step 1: 2-Chloro-3-(4-methylpiperazin-1-yl)phenol

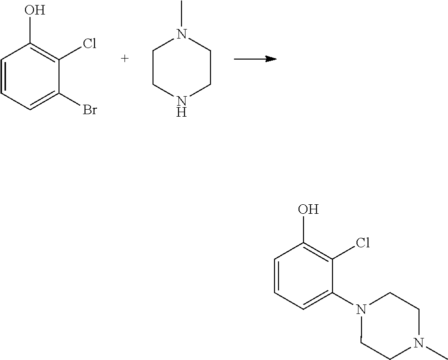

A heterogeneous mixture of 3-bromo-2-chloro-phenol (4.20 g, 20.25 mmol), 1-methylpiperazine (21.5 g, 214.7 mmol), chloro(2-di-t-butylphosphino-2′, 4′, 6′-tri-i-propyl-1,1′-biphenyl)[2-(2-aminoethyl)phenyl] palladium(II) [t-BuXPhos Palladacycle Gen. 1] (2.1 g, 3.058 mmol), and potassium tert-butoxide (4.8 g, 42.78 mmol) in dioxane (120 mL) was sonicated for 15 minutes and heated in a sealed vessel at 50° C. for 16 hours. The reaction mixture was acidified using acetic acid (3.4 mL, 59.79 mmol) then partitioned between DCM (100 mL) and water (100 mL). The organic layer was separated, and the aqueous layer was further extracted with DCM (4×). The combined organics were washed once with brine, dried using magnesium sulfate, filtered, and concentrated in vacuo. The crude residue was separated by flash column chromatography on silica gel (gradient: 1 to 10% methanol in dichloromethane), to afford 2-chloro-3-(4-methylpiperazin-1-yl)phenol (3.86 g, 84%) as a yellow solid. ESI-MS m/z calc. 226.0873, found 227.12 (M+1)⁺; Retention time: 0.24 minutes. (LC method D).

### Step 2: N-[4-[2-chloro-3-(4-methylpiperazin-1-yl)phenoxy]-6-(2,6-dimethylphenyl)pyrimidin-2-yl]-1-methyl-pyrazole-4-sulfonamide

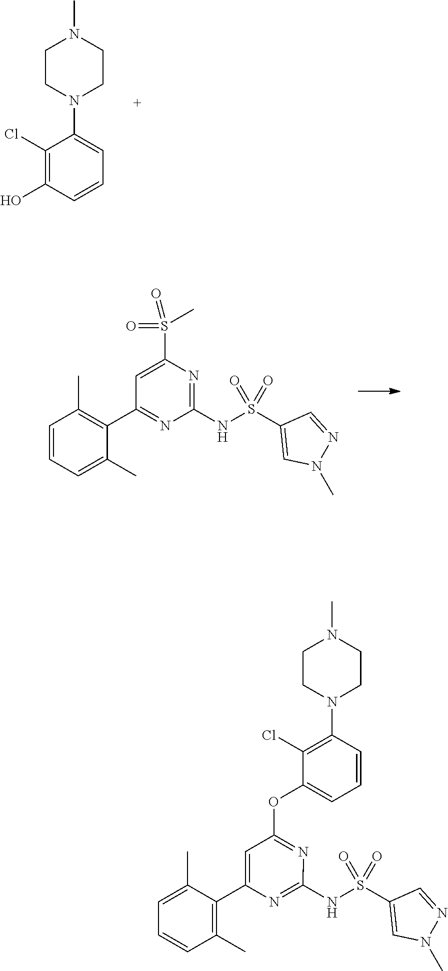 [SMILES: CN1CCN(c2cccc(O)c2Cl)CC1 ; Cc1cccc(C)c1-c1cc(S(C)(=O)=O)nc(NS(=O)(=O)c2cnn(C)c2)n1 ; Cc1cccc(C)c1-c1cc(Oc2cccc(N3CCN(C)CC3)c2Cl)nc(NS(=O)(=O)c2cnn(C)c2)n1]

An NMP (0.6 mL) mixture of 2-chloro-3-(4-methylpiperazin-1-yl)phenol (approximately 17.91 mg, 0.07899 mmol), Cs₂CO₃ (approximately 34.31 mg, 0.1053 mmol)N-[4-(2,6-dimethylphenyl)-6-methylsulfonyl-pyrimidin-2-yl]-1-methyl-pyrazole-4-sulfonamide (11.1 mg, 0.02633 mmol) was heated to 110° C. for 2 hours and then cooled to room temperature. The solution was filtered, and the resulting residue diluted with 0.8 mL MeOH, and purified by reverse phase chromatography using a 15 minute gradient of 20% MeCN in water to 80% MeCN (HCl modifier) to give the title compound. N-[4-[2-chloro-3-(4-methylpiperazin-1-yl)phenoxy]-6-(2,6-dimethylphenyl)pyrimidin-2-yl]-1-methyl-pyrazole-4-sulfonamide (7.9 mg, 46%). ESI-MS m/z calc. 567.18195, found 568.53 (M+1)⁺; Retention time: 1.29 minutes; LC method A.

### Example 279: Preparation of Compound 921

### Step 1: 2-Chloro-5-(4-methylpiperazin-1-yl)phenol

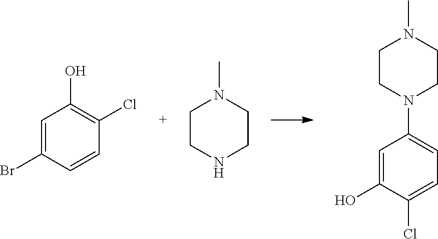

The compound was prepared in a manner analogous to that described above using commercially available 5-bromo-2-chloro-phenol (350 mg, 1.687 mmol) and -methylpiperazine (approximately 1.690 g, 1.874 mL, 16.87 mmol) to give 2-chloro-5-(4-methylpiperazin-1-yl)phenol. ESI-MS m/z calc. 226.0873, found 227.29 (M+1)⁺; Retention time: 0.64 minutes; LC method A. ¹H NMR (400 MHz, Chloroform-d) δ 7.14 (d, J=8.9 Hz, 1H), 6.56 (d, J=2.8 Hz, 1H), 6.45 (dd, J=8.9, 2.9 Hz, 1H), 3.22-3.13 (m, 4H), 2.60-2.52 (m, 5H), 2.34 (s, 3H).

### Step 2: N-[4-[2-chloro-5-(4-methylpiperazin-1-yl)phenoxy]-6-(2,6-dimethylphenyl)pyrimidin-2-yl]-1-methyl-pyrazole-4-sulfonamide

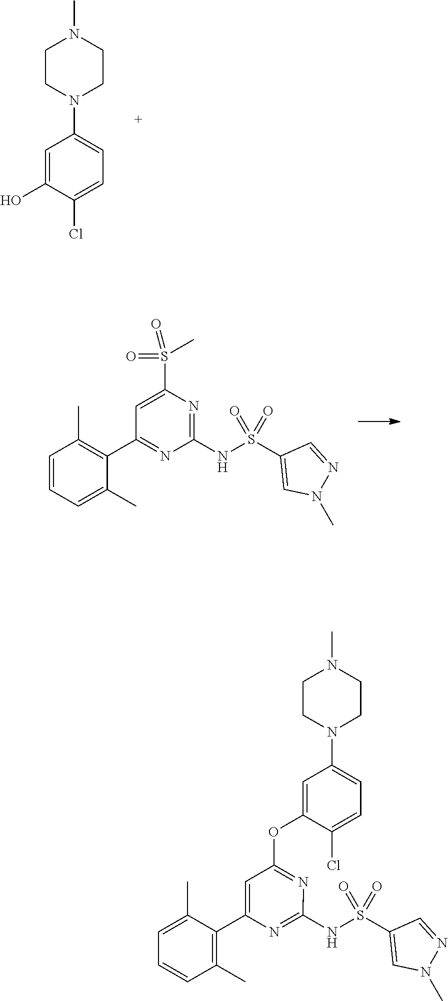 [SMILES: CN1CCN(c2ccc(Cl)c(O)c2)CC1 ; Cc1cccc(C)c1-c1cc(S(C)(=O)=O)nc(NS(=O)(=O)c2cnn(C)c2)n1 ; Cc1cccc(C)c1-c1cc(Oc2cc(N3CCN(C)CC3)ccc2Cl)nc(NS(=O)(=O)c2cnn(C)c2)n1]

A mixture of 2-chloro-5-(4-methylpiperazin-1-yl)phenol (approximately 21.62 mg, 0.09537 mmol), Cs₂CO₃ (approximately 41.44 mg, 0.1272 mmol) and N-[4-(2,6-dimethylphenyl)-6-methylsulfonyl-pyrimidin-2-yl]-1-methyl-pyrazole-4-sulfonamide (13.4 mg, 0.03179 mmol) in NMP (0.6 mL) was heated to 110° C. for 2 hours and then cooled to room temperature. The solution was filtered, and the resulting residue diluted with 0.8 mL MeOH, and purified by reverse phase chromatography using a 15 min gradient of 20% MeCN in water to 80 % MeCN (HCl modifier) to give the title compound. N-[4-[2-chloro-5-(4-methylpiperazin-1-yl)phenoxy]-6-(2,6-dimethylphenyl)pyrimidin-2-yl]-1-methyl-pyrazole-4-sulfonamide (8.8 mg, 37%). ESI-MS m/z calc. 567.18195, found 568.53 (M+1)⁺; Retention time: 1.33 minutes; LC method A.

### Example 280: Preparation of Compound 922

### Step 1: 3-(4-methylpiperazin-1-yl)-4-(trifluoromethoxy)phenol

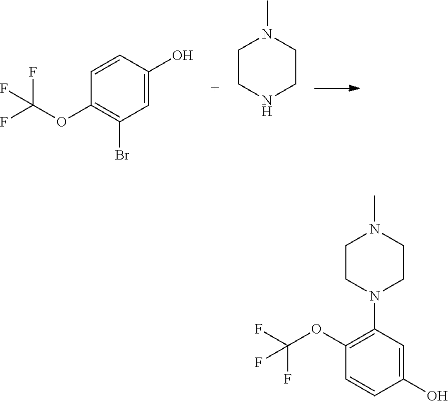

The compound was prepared in a manner analogous to that described above using commercially available 3-bromo-4-(trifluoromethoxy)phenol (46.4 mg, 0.1805 mmol) and 1-methylpiperazine (approximately 54.24 mg, 60.13 μL, 0.5415 mmol) to give 3-(4-methylpiperazin-1-yl)-4-(trifluoromethoxy)phenol.

### Step 2: N-[4-(2,6-Dimethylphenyl)-6-[3-(4-methylpiperazin-1-yl)-4-(trifluoromethoxy)phenoxy]pyrimidin-2-yl]-1-methyl-pyrazole-4-sulfonamide

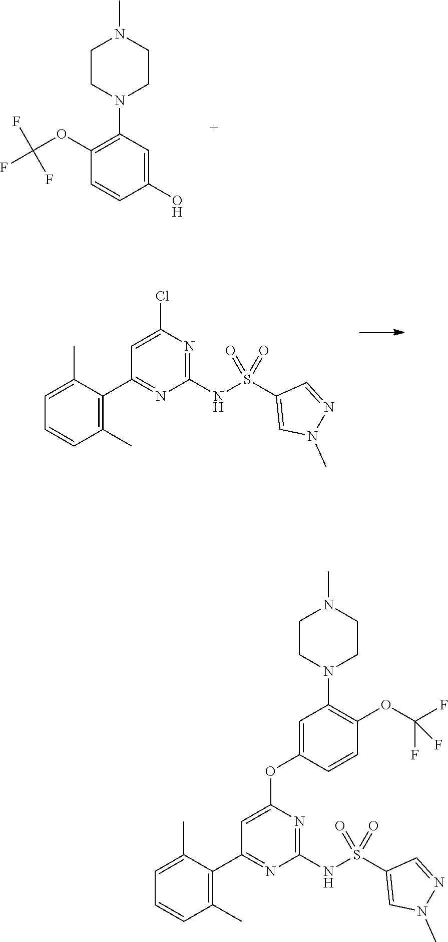 [SMILES: CN1CCN(c2cc(O)ccc2OC(F)(F)F)CC1 ; Cc1cccc(C)c1-c1cc(Cl)nc(NS(=O)(=O)c2cnn(C)c2)n1 ; Cc1cccc(C)c1-c1cc(Oc2ccc(OC(F)(F)F)c(N3CCN(C)CC3)c2)nc(NS(=O)(=O)c2cnn(C)c2)n1]

A mixture of 3-(4-methylpiperazin-1-yl)-4-(trifluoromethoxy)phenol (hydrochloride salt) (14.1 mg, 0.04509 mmol), N-[4-chloro-6-(2,6-dimethylphenyl)pyrimidin-2-yl]-1-methyl-pyrazole-4-sulfonamide (11.6 mg, 0.03070 mmol), and Cs₂CO₃ (54.3 mg, 0.1667 mmol) in NMP (0.6 mL)was stirred at 110° C. for 16 hours and then cooled to room temperature. The solution was filtered and the filtrate dissolved in 0.8 mL MeOH, and purified by reverse phase chromatography using a 15 min gradient of 1% MeCN in water to 99% MeCN (HCl modifier) to give N-[4-(2,6-dimethylphenyl)-6-[3-(4-methylpiperazin-1-yl)-4-(trifluoromethoxy)phenoxy]pyrimidin-2-yl]-1-methyl-pyrazole-4-sulfonamide (hydrochloride salt) (3.7 mg, 18%). ESI-MS m/z calc. 617.2032, found 618.2 (M+1)⁺; Retention time: 1.36 minutes (LC method A).

### Example 281: Preparation of Compound 923

### Step 1: 3-(4-Methylpiperazin-1-yl)phenol

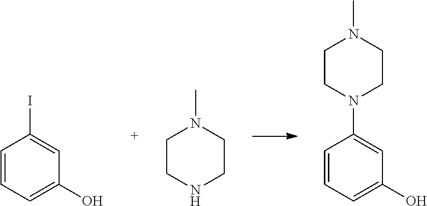

A heterogeneous mixture of 3-iodophenol (2.4 g, 10.91 mmol), 1-methylpiperazine (approximately 10.93 g, 109.1 mmol), potassium tert-butoxide (approximately 2.571 g, 22.91 mmol), and Chloro(2-di-t-butylphosphino-2′, 4′, 6′-tri-i-propyl-1,1′-biphenyl)[2-(2-aminoethyl)phenyl] palladium(II) [t-BuXPhos Palladacycle Gen. 1] (approximately 374.6 mg, 0.5455 mmol) in dioxane (64.18 mL) was heated to 60° C. for 16 hours. The crude mixture was concentrated in vacuo. The crude residue was dissolved in acetic acid (approximately 39.31 g, 37.23 mL, 654.6 mmol) and concentrated in vacuo onto silica gel. The crude impregnated silica gel was separated by flash column chromatography (gradient: 1 to 10% MeOH in DCM) to afford 3-(4-methylpiperazin-1-yl)phenol (1.27 g, 58%) as a dark red solid. ESI-MS m/z calc. 192.12627, found 193.08 (M+1)⁺; Retention time: 0.17 minutes; LC method D.

### Step 2: N-[4-(2,6-Dimethylphenyl)-6-[3-(4-methylpiperazin-1-yl)phenoxy]pyrimidin-2-yl]-1-methyl-pyrazole-4-sulfonamide

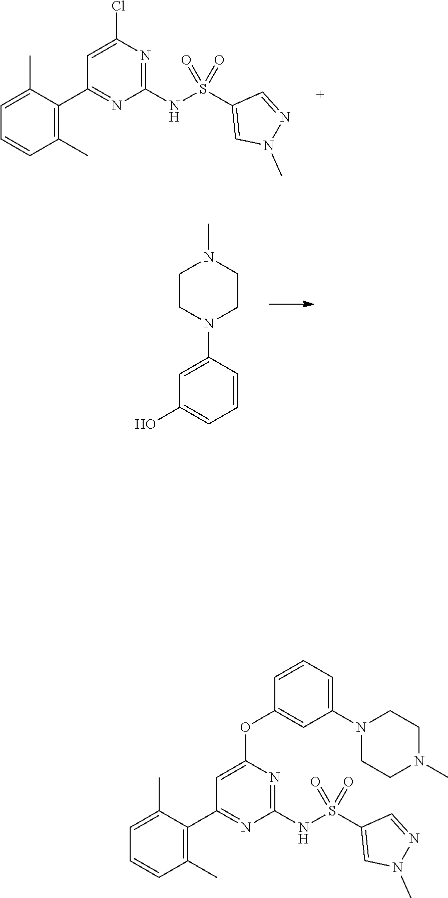 [SMILES: Cc1cccc(C)c1-c1cc(Cl)nc(NS(=O)(=O)c2cnn(C)c2)n1 ; CN1CCN(c2cccc(O)c2)CC1 ; Cc1cccc(C)c1-c1cc(Oc2cccc(N3CCN(C)CC3)c2)nc(NS(=O)(=O)c2cnn(C)c2)n1]

A mixture of N-[4-chloro-6-(2,6-dimethylphenyl)pyrimidin-2-yl]-1-methyl-pyrazole-4-sulfonamide (83 mg, 0.2153 mmol), 3-(4-methylpiperazin-1-yl)phenol (hydrochloride salt) (70 mg, 0.3061 mmol) and Cs₂CO₃ (320 mg, 0.9821 mmol) in NMP (1 mL) was stirred at 100° C. for 4 hours. The reaction mixture was diluted with MeOH, filtered and purification by HPLC (1-99% ACN in water (HCl modifier)) gave N-[4-(2,6-dimethylphenyl)-6-[3-(4-methylpiperazin-1-yl)phenoxy]pyrimidin-2-yl]-1-methyl-pyrazole-4-sulfonamide (hydrochloride salt) (45 mg, 37%). ESI-MS m/z calc. 533.2209, found 534.3 (M+1)⁺; Retention time: 1.25 minutes (LC method A).

### Example 282: Preparation of Compound 924

### Step 1: 4-chloro-3-(4-methylpiperazin-1-yl)phenol

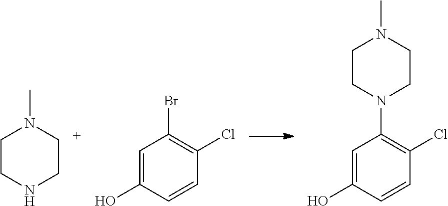

A dioxane (12 mL) solution of 3-bromo-4-chloro-phenol (298.9 mg, 1.441 mmol) [2-(2-aminoethyl)phenyl]-chloro-palladium;ditert-butyl-[2-(2,4,6-triisopropylphenyl)phenyl]phosphane (XPhos Pd G1) (approximately 197.9 mg, 0.2882 mmol), 1-methylpiperazine (approximately 1.443 g, 14.41 mmol), and sodium tert-butoxide (343.7 mg, 3.576 mmol) was sparged with nitrogen at room temperature for 15 minutes and then heated at 50° C. for 16 hours. The reaction is then cooled to room temperature and poured into a saturated aqueous solution of ammonium chloride (25 mL) and dichloromethane (60 mL). The two layers are separated, and the organic layer is dried with anhydrous sodium sulfate, filtered, concentrated in vacuo and purified by flash column chromatography (12 g silica) using a 15minute gradient of 0%-10% MeOH in DCM to give 4-chloro-3-(4-methylpiperazin-1-yl)phenol (236 mg, 72%)¹H NMR (400 MHz, Chloroform-d) δ 7.17 (d, J=8.6 Hz, 1H), 6.67 (d, J=2.8 Hz, 1H), 6.47 (dd, J=8.6, 2.8 Hz, 1H), 3.10 (s, 5H), 2.69 (s, 5H), 2.40 (s, 3H). ESI-MS m/z calc. 226.0873, found 227.29 (M+1)⁺; Retention time: 0.68 minutes (LC method D).

### Step 2: N-[4-[4-chloro-3-(4-methylpiperazin-1-yl)phenoxy]-6-(2,6-dimethylphenyl)pyrimidin-2-yl]-1-methyl-pyrazole-4-sulfonamide

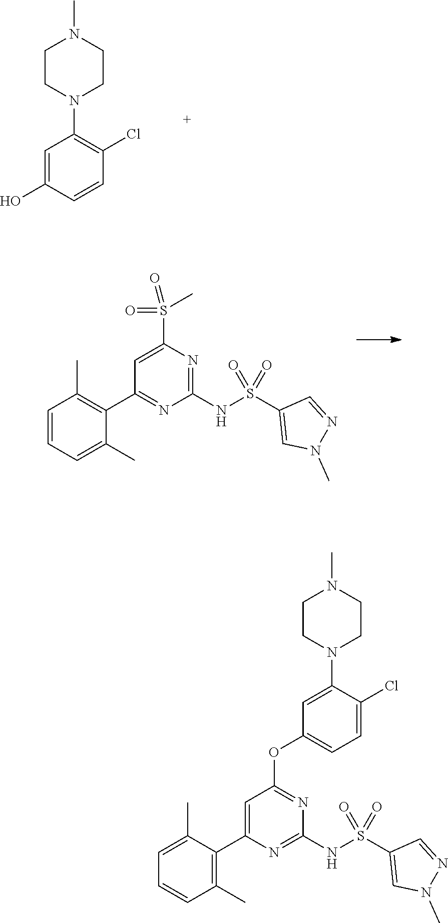 [SMILES: CN1CCN(c2cc(O)ccc2Cl)CC1 ; Cc1cccc(C)c1-c1cc(S(C)(=O)=O)nc(NS(=O)(=O)c2cnn(C)c2)n1 ; Cc1cccc(C)c1-c1cc(Oc2ccc(Cl)c(N3CCN(C)CC3)c2)nc(NS(=O)(=O)c2cnn(C)c2)n1]

A mixture of 4-chloro-3-(4-methylpiperazin-1-yl)phenol (approximately 24.21 mg, 0.1068 mmol), Cs₂CO₃ (approximately 46.40 mg, 0.1424 mmol)N-[4-(2,6-dimethylphenyl)-6-methylsulfonyl-pyrimidin-2-yl]-1-methyl-pyrazole-4-sulfonamide (15 mg, 0.03559 mmol) in NMP (0.6 mL) was heated to 110° C. for 2 hours and then cooled to room temperature. The mixture was filtered, and the resulting residue diluted with 0.8 mL MeOH, and purified by reverse phase chromatography using a 15 minute gradient of 20% MeCN in water to 80% MeCN (HCl modifier) to give the title compound. N-[4-[4-chloro-3-(4-methylpiperazin-1-yl)phenoxy]-6-(2,6-dimethylphenyl)pyrimidin-2-yl]-1-methyl-pyrazole-4-sulfonamide (7.2 mg, 28%). ESI-MS m/z calc. 567.18195, found 568.53 (M+1)⁺; Retention time: 1.29 minutes; LC method A.

### Example 283: Preparation of Compound 925

### Step 1: 2-Fluoro-3-(4-methylpiperazin-1-yl)phenol

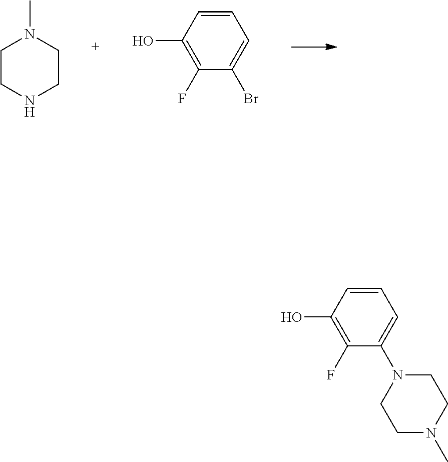

A dioxane (15 mL) mixture of 1-methylpiperazine (950 μL, 8.555 mmol), 3-bromo-2-fluoro-phenol (534.7 mg, 2.800 mmol), sodium tert-butoxide (1.046 g, 10.88 mmol), and chloro(2-di-t-butylphosphino-2′, 4′, 6′-tri-i-propyl-1,1′-biphenyl)[2-(2-aminoethyl)phenyl]palladium(II) [t-BuXPhos Palladacycle Gen. 1] (365.1 mg, 0.5606 mmol) was stirred at room temperature for 2 hours and then HCl (10 mL of 1 M, 10.00 mmol) was added to bring the pH of the aqueous layer to ˜7-8. Ethyl acetate (20 mL) was added and the two layers are separated. The product was extracted from the aqueous layer with ethyl acetate (2×10 mL) and the combined organic layers were dried with anhydrous sodium sulfate, filtered, and concentrated in vacuo. The material was taken up in minimal dichloromethane and loaded onto a 24 g column and the compound is purified by flash column chromatography with a gradient of 0-10% MeOH/DCM over 20 minutes to give 2-fluoro-3-(4-methylpiperazin-1-yl)phenol (321.2 mg, 55%) ESI-MS m/z calc. 210.11684, found 211.13 (M+1)⁺; Retention time: 0.49 minutes (LC method A).

### Step 2: N-[4-(2,6-Dimethylphenyl)-6-[2-fluoro-3-(4-methylpiperazin-1-yl)phenoxy]pyrimidin-2-yl]-1-methyl-pyrazole-4-sulfonamide

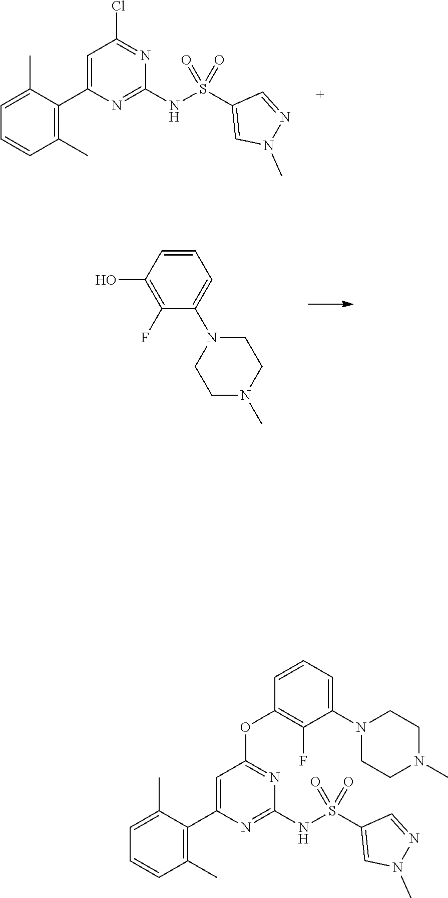 [SMILES: Cc1cccc(C)c1-c1cc(Cl)nc(NS(=O)(=O)c2cnn(C)c2)n1 ; CN1CCN(c2cccc(O)c2F)CC1 ; Cc1cccc(C)c1-c1cc(Oc2cccc(N3CCN(C)CC3)c2F)nc(NS(=O)(=O)c2cnn(C)c2)n1]

A mixture of N-[4-chloro-6-(2,6-dimethylphenyl)pyrimidin-2-yl]-1-methyl-pyrazole-4-sulfonamide (21.4 mg, 0.05664 mmol), 2-fluoro-3-(4-methylpiperazin-1-yl)phenol (51.2 mg, 0.2435 mmol), and Cs₂CO₃ (80.3 mg, 0.2465 mmol) in NMP (0.5 mL) was stirred at 110° C. for 16 hours and then cooled to room temperature. The solution was filtered and the filtrate dissolved in 0.8 mL MeOH, and purified by reverse phase chromatography using a 15 minutes gradient of 20% MeCN in water to 80% MeCN (HCl modifier) to give N-[4-(2,6-dimethylphenyl)-6-[2-fluoro-3-(4-methylpiperazin-1-yl)phenoxy]pyrimidin-2-yl]-1-methyl-pyrazole-4-sulfonamide (hydrochloride salt) (4.6 mg, 3%) ESI-MS m/z calc. 551.2115, found 552.1 (M+1)⁺; Retention time: 1.17 minutes (LC method A).

### Example 284: Preparation of Compound 926

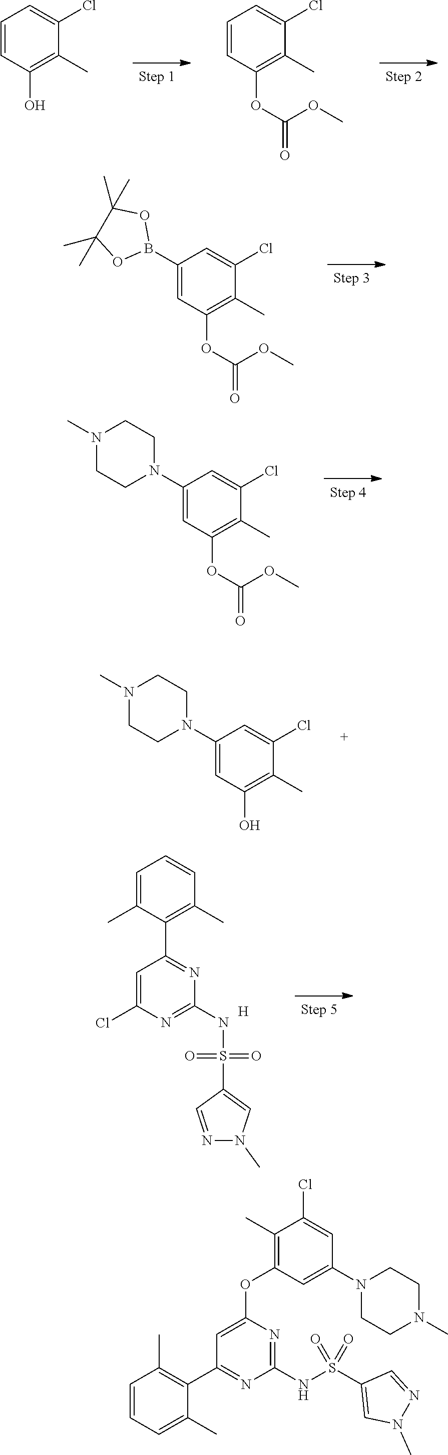

### Step 1: 3-Chloro-2-methylphenyl methyl carbonate

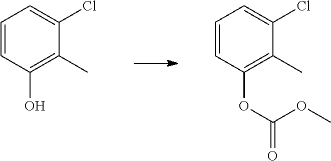

To a solution of 3-chloro-2-methyl-phenol (66.78 g, 0.46 mol) and triethylamine (93.42 mL, 0.67 mol) in tetrahydrofuran (1.34 L) and was added slowly methyl chloroformate (38.00 mL, 0.49 mol) at 0° C. The reaction mixture was stirred at ambient temperature for 3 hours. The formed precipitate was filtered off and the filtrate concentrated under reduced pressure. Diethyl ether (500 mL) and water (500 mL) were added, and the aqueous layer extracted with diethyl ether (2×500 mL). The combined organics were dried over sodium sulfate and concentrated to afford 3-chloro-2-methylphenyl methyl carbonate (83.19 g, 89%) as an amber glass. ¹H NMR (250 MHz, CDCl₃) δ (ppm): 7.29 (d, 1H, J=7.8 Hz), 7.16 (t, 1H, J=8.1 Hz), 7.05 (d, 1H, J=8.0 Hz), 3.92 (s, 3H), 2.27 (s, 3H). ESI-MS m/z calc. 200.62, found 201.3 (M+1). Retention time: 3.11 minutes.

### Step 2: 3-Chloro-2-methyl-5-(4,4,5,5-tetramethyl-1,3,2-dioxaborolan-2-yl)phenyl methyl carbonate

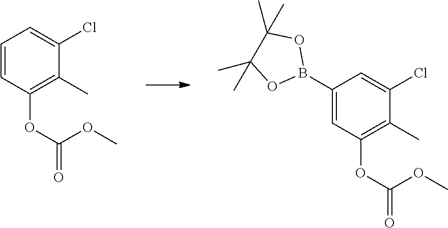

A sealed vessel was charged with 3-chloro-2-methylphenyl methyl carbonate (25.00 g, 124.6 mmol) and bis(pinacolato)diboron (34.81 g, 137.1 mmol), di-mu-methoxobis(1,5-cyclooctadiene)diiridium (826 mg, 1.25 mmol), 4,4′-di-tert-butyl-2,2′-dipyridyl (669 mg, 2.49 mmol) and anhydrous tetrahydrofuran (250 mL) and the reaction mixture was stirred at 80° C. for 16 hours. The reaction mixture was concentrated under reduced pressure and the residue was purified by silica gel column chromatography using 0-10% hexanes-diethyl ether to afford 3-chloro-2-methyl-5-(4,4,5,5-tetramethyl-1,3,2-dioxaborolan-2-yl)phenyl methyl carbonate (26.18 g, 64%) as an off-white solid. ¹H NMR (250 MHz, CDCl₃) δ (ppm): 7.70 (s, 1H), 7.43 (s, 1H), 3.91 (s, 3H), 2.28 (s, 3H), 1.32 (s, 12H). ESI-MS m/z calc. 326.59, found 327.5 (M+1). Retention time: 3.95 minutes.

### Step 3: 3-Chloro-2-methyl-5-(4-methylpiperazin-1-yl)phenyl methyl carbonate

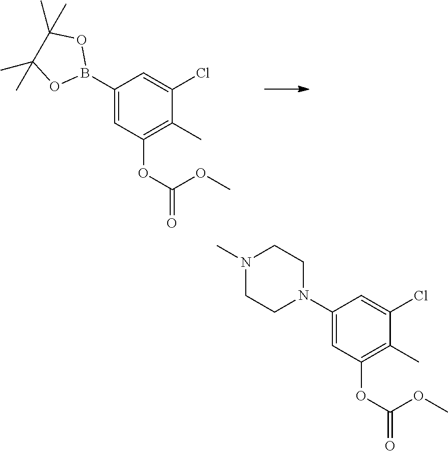

A sealed vessel was charged with -chloro-2-methyl-5-(4,4,5,5-tetramethyl-1,3,2-dioxaborolan-2-yl)phenyl methyl carbonate (7.00 g, 21.43 mmol), copper acetate (3.89 g, 21.43 mmol), 4 Å molecular sieves (8.50 g), 1-methylpiperazine (2.88 mL, 26.00 mmol), anhydrous pyridine (3.44 mL, 42.58 mmol) and anhydrous dichloromethane (84.0 mL) and the reaction stirred for 16 hours at room temperature. The molecular sieves were filtered off and the reaction mixture was concentrated under reduced pressure. The residue was purified by silica gel column chromatography using 0-10% methanol in dichloromethane to afford 3-chloro-2-methyl-5-(4-methylpiperazin-1-yl)phenyl methyl carbonate (1.15 g, 18%) as an amber oil. ¹H NMR (250 MHz, CDCl₃) δ (ppm): 6.83 (d, 1H, J=2.4 Hz), 6.57 (d, 1H, J=2.3 Hz), 3.90 (s, 3H), 3.27-3.18 (m, 4H), 2.76-2.66 (m, 4H), 2.41 (s, 3H), 2.15 (s, 3H). ESI-MS m/z calc. 298.77, found 299.1 (M+1). Retention time: 2.11 minutes.

### Step 4: 3-Chloro-2-methyl-5-(4-methyl-piperazin-1-yl)-phenol

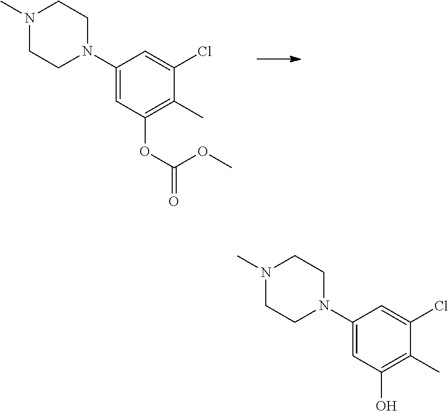

A solution of 3-chloro-2-methyl-5-(4-methylpiperazin-1-yl)phenyl methyl carbonate (1.15 g, 3.85 mmol) and piperidine (0.95 mL, 9.62 mmol) in dichloromethane (38.5 mL) was stirred at 40° C. for 18 hours. The reaction mixture was diluted with dichloromethane (60 mL) and extracted with 1 N aqueous sodium hydroxide solution (3×35 mL). 1 N aqueous hydrogen chloride solution was added to the combined aqueous layer to adjust pH to 9-10 and the aqueous phase was extracted with dichloromethane (3×75 mL). The combined organics were dried over sodium sulfate and concentrated under reduced pressure to afford 3-chloro-2-methyl-5-(4 -methyl-piperazin-1-yl)-phenol (213 mg, 23%) as a tan solid. ¹H NMR (250 MHz, CDCl₃) δ (ppm): 6.54 (d, 1H, J=2.4 Hz), 6.26 (d, 1H, J=2.3 Hz), 3.17-3.09 (m, 4H), 2.61-2.52 (m, 4H), 2.36 (s, 3H), 2.19 (s, 3H). ESI-MS m/z calc. 240.74, found 241.2 (M+1). Retention time: 1.89 minutes.

### Step 5: N-[4-[3-chloro-2-methyl-5-(4-methylpiperazin-1-yl)phenoxy]-6-(2,6-dimethylphenyl)pyrimidin-2-yl]-1-methyl-pyrazole-4-sulfonamide

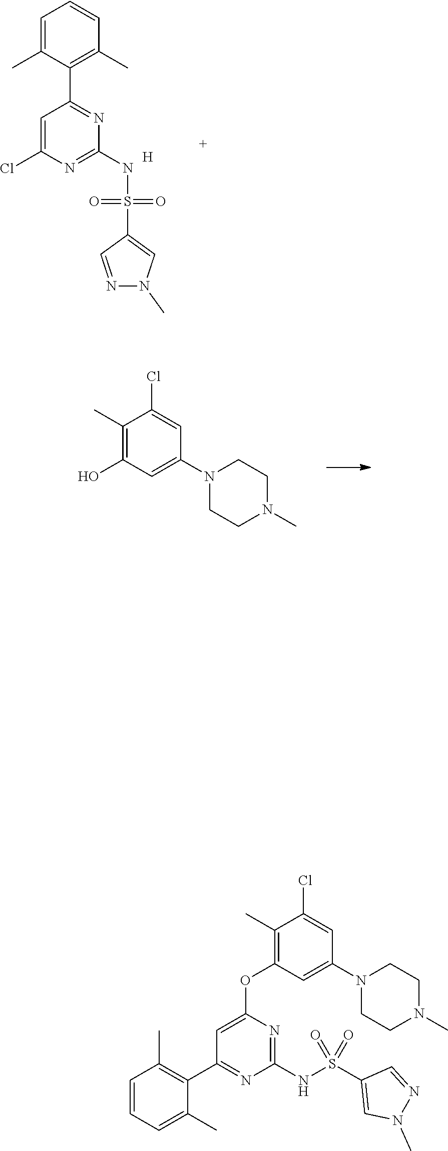

To a mixture of N-[4-chloro-6-(2,6-dimethylphenyl)pyrimidin-2-yl]-1-methyl-pyrazole-4-sulfonamide (37.78 mg, 0.1 mmol) and Cs₂CO₃ (approximately 130.3 mg, 0.4000 mmol) in NMP (0.4 mL) was added 3-chloro-2-methyl-5-(4-methylpiperazin-1-yl)phenol (approximately 72.22 mg, 0.3000 mmol) and the reaction mixture was stirred at 120° C. for 16 hours. The reaction mixture was diluted with MeOH, filtered and purification by HPLC (1-99% ACN in water (HCl modifier)) gave N-[4-[3-chloro-2-methyl-5-(4-methylpiperazin-1-yl)phenoxy]-6-(2,6-dimethylphenyl)pyrimidin-2-yl]-1-methyl-pyrazole-4-sulfonamide (hydrochloride salt) (35.1 mg, 57%). ESI-MS m/z calc. 581.1976, found 582.3 (M+1)⁺; Retention time: 1.29 minutes; LC method A.

### Example 285: Preparation of Compound 927

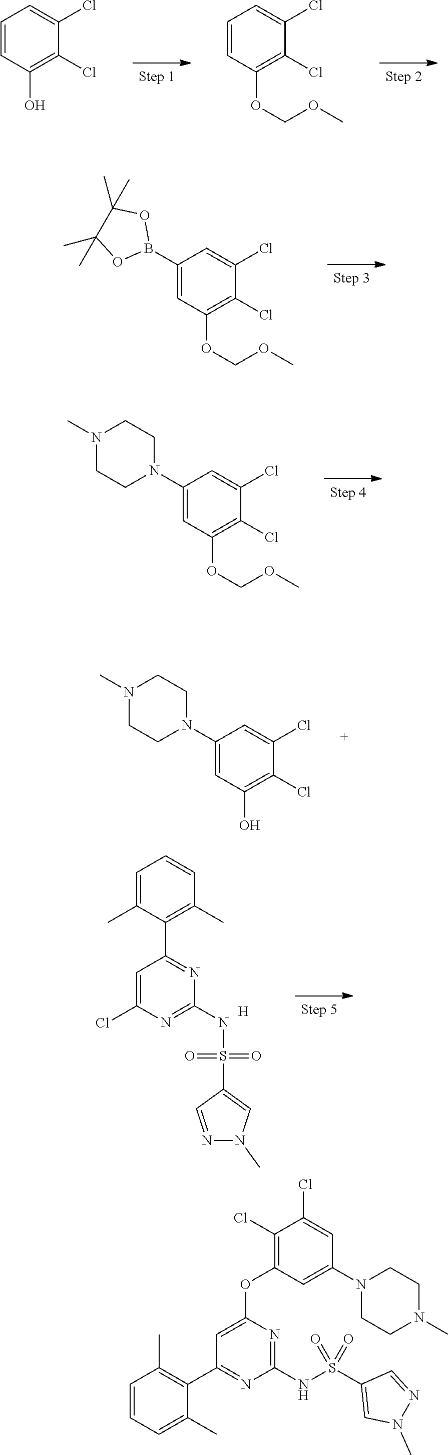

### Step 1: 1,2-Dichloro-3-methoxymethoxy-benzene

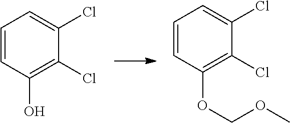

To a suspension of 2,3-dichlorophenol (15.12 g, 92.76 mmol) and potassium carbonate (25.64 g, 185.52 mmol) in acetonitrile (140 mL) was added chloromethyl methyl ether (8.46 mL, 111.34 mmol) and the reaction mixture was stirred at 60° C. for 3 hours. The reaction mixture was concentrated and dichloromethane (250 mL) and water (250 mL) were added. The aqueous phase was extracted with dichloromethane (2×200 mL). The combined organics were dried over sodium sulfate and concentrated to afford 1,2-dichloro-3-methoxymethoxy-benzene (16.47 g, 86%) as a clear liquid. ¹H NMR (250 MHz, CDCl₃) δ (ppm): 7.15-7.03 (m, 3H), 5.26 (s, 2H), 3.52 (s, 3H)

### Step 2: 2-(3,4-Dichloro-5-methoxymethoxy-phenyl)-4,4,5,5-tetramethyl-[1,3,2]dioxaborolane

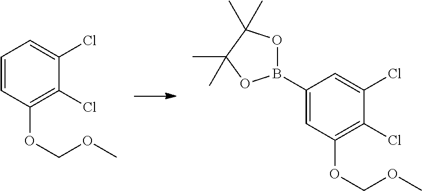

A sealed vessel was charged with 1,2-dichloro-3-methoxymethoxy-benzene (16.47 g, 79.54 mmol), bis(pinacolato)diboron (22.33 g, 87.93 mmol),di--methoxobis(1,5-cyclooctadiene)diiridium (530 mg, 0.80 mmol, 4,4′-di-tert-butyl-2,2′-dipyridyl (430 mg, 0.16 mmol) and anhydrous tetrahydrofuran (165 mL) and the reaction mixture was stirred at 80° C. for 16 hours. The reaction mixture was concentrated under reduced pressure and the residue was purified by silica gel column chromatography using 0-10% hexanes-diethyl ether to afford 2-(3,4-dichloro-5-methoxymethoxy-phenyl)-4,4,5,5-tetramethyl-[1,3,2]dioxaborolane (14.57 g, 55%) as a white solid. ¹H NMR (250 MHz, CDCl₃) δ (ppm): 7.56 (s, 1H), 7.42 (s, 1H), 5.30 (s, 2H), 3.53 (s, 3H), 1.33 (s, 12H) ESI-MS m/z calc. 333.02, found 333.4 (M+1). Retention time: 3.69 minutes.

### Step 3: 1-(3,4-Dichloro-5-methoxymethoxy-phenyl)-4-methyl-piperazine

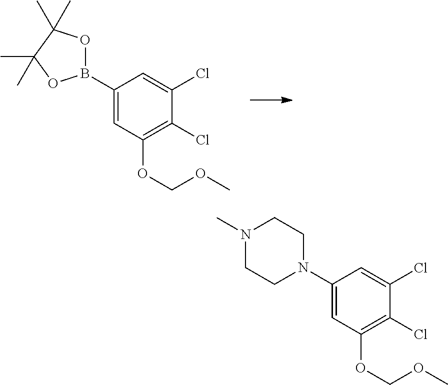

2-(3,4-Dichloro-5-methoxymethoxy-phenyl)-4,4,5,5-tetramethyl-[1,3,2]dioxaborolane (3.00 g, 9.01 mmol), 1-methylpiperazine (1.68 mL, 15.10 mmol) and 4 Å molecular sieves (2.50 g) were charged into a sealed vessel and anhydrous dichloromethane (65 mL) was added. The solution was stirred for 15 minutes, then 3-hexyne (2.27 mL, 20.01 mmol), copper acetate (1.82 g, 10.02 mmol), and anhydrous pyridine (1.62 mL, 20.02 mmol) were added sequentially. The reaction mixture was placed into a preheated 55° C. oil bath and stirred at 55° C. for 41 hours. The reaction mixture was concentrated under reduced pressure and the crude residue purified by silica gel column chromatography using 0-5% dichloromethane-methanol gradient to afford 1-(3,4-dichloro-5-methoxymethoxy-phenyl)-4-methyl-piperazine (571 mg, 21%) as a clear liquid.

¹H NMR (250 MHz, CDCl₃) δ (ppm): 6.67 (s, 1H), 5.22 (s, 2H), 3.52 (s, 3H), 3.22-3.12 (m, 4H), 2.58-2.49 (m, 4H), 2.34 (s, 3H). ESI-MS m/z calc. 305.21, found 305.3 (M+1). Retention time: 2.13 minutes.

### Step 4: 2,3-Dichloro-5-(4-methyl-piperazin-1-yl)-phenol

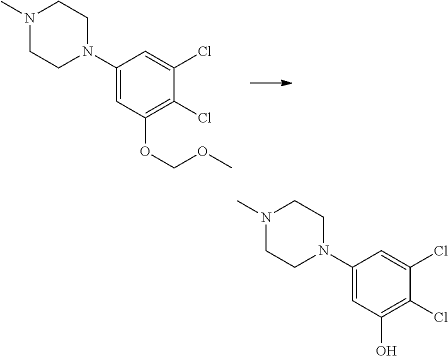

To a solution of 1-(3,4-dichloro-5-methoxymethoxy-phenyl)-4-methyl-piperazine (0.57 g, 1.87 mmol) in dichloromethane (20 mL) was added trifluoroacetic acid (2.5 mL) and the reaction solution was stirred at ambient temperature for 18 hours. The volatiles were removed under reduced pressure and the residue dissolved in water (10 mL). 5% aqueous sodium bicarbonate solution (70 mL) was added and the aqueous layer was extracted with chloroform (3×75 mL). The organic phase was dried over sodium sulfate and concentrated to afford 2,3-dichloro-5-(4-methyl-piperazin-1-yl)-phenol (265 mg, 53.8% ) as a brown foam. ¹H NMR (250 MHz, CDCl₃) δ (ppm): 6.60 (d, 1H, J=2.8 Hz), 6.47 (d, 1H, J=2.5 Hz), 3.23-3.14 (m, 4H), 2.60-2.51 (m, 4H), 2.35 (s, 3H). ESI-MS m/z calc. 261.15, found 262.6 (M+1). Retention time: 2.04 minutes.

### Step 5: N-[4-[2,3-Dichloro-5-(4-methylpiperazin-1-yl)phenoxy]-6-(2,6-dimethylphenyl)pyrimidin-2-yl]-1-methyl-pyrazole-4-sulfonamide

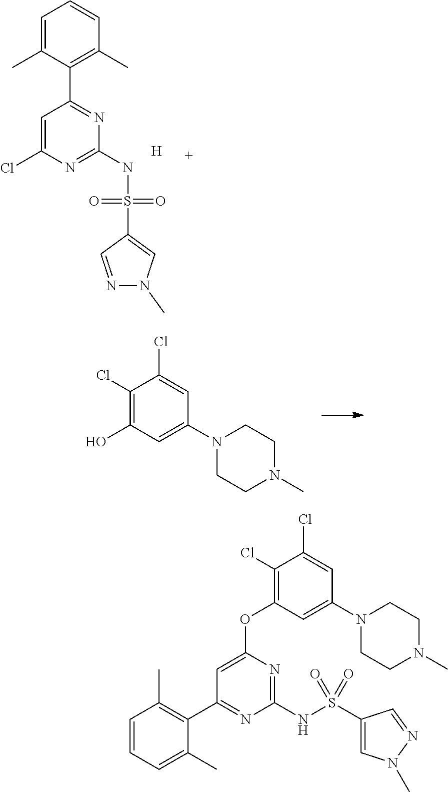

To a mixture of N-[4-chloro-6-(2,6-dimethylphenyl)pyrimidin-2-yl]-1-methyl-pyrazole-4-sulfonamide (37.78 mg, 0.1 mmol) and Cs₂CO₃ (approximately 130.3 mg, 0.4000 mmol) in NMP (0.4 mL) was added 2,3-dichloro-5-(4-methylpiperazin-1-yl)phenol (approximately 78.34 mg, 0.3000 mmol) and the reaction mixture stirred at 120° C. for 16 hours. The reaction mixture was diluted with MeOH, filtered and purification by HPLC (1-99% ACN in water (HCl modifier)) gave N-[4-[2,3-dichloro-5-(4-methylpiperazin-1-yl)phenoxy]-6-(2,6-dimethylphenyl)pyrimidin-2-yl]-1-methyl-pyrazole-4-sulfonamide (hydrochloride salt) (15.2 mg, 23%). ESI-MS m/z calc. 601.14294, found 602.2 (M+1)⁺; Retention time: 1.3 minutes; LC method A.

### Example 286: Preparation of Compound 928

### Step 1: 3-(1-Methyl-2-piperidyl)phenol

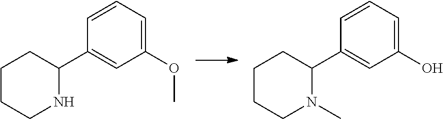

A room temperature solution of 2-(3-methoxyphenyl)piperidine (162.8 mg, 0.8512 mmol) in MeOH (2000 μL) and DCM (3 mL) was treated with formaldehyde (1500 μL, 54 mmol)(37% in water), formic acid (1500 μL, 40. mmol), and sodium triacetoxyborohydride (322.3 mg, 1.521 mmol). The mixture was stirred for 16 hours at 60° C. and then concentrated in vacuo. The resulting residue was taken up in concentrated hydrobromic acid (1000 μL, 18.42 mmol) and heated at 100° C. for 16 hours and then concentrated in vacuo. The residue was dissolved in DMSO/MeOH (1:1) (1.5 mL) and the solution was filtered and purified by reverse phase chromatography using a 15 minute gradient of 1% MeCN in water to 50% MeCN (HCl modifier) to give 3-(1-methyl-2-piperidyl)phenol (hydrochloride salt) (18.0 mg, 9%) ESI-MS m/z calc. 191.13101, found 192.27 (M+1)⁺; Retention time: 0.54 minutes. (LC method A).

### Step 2: N-[4-(2,6-Dimethylphenyl)-6-[3-(1-methyl-2-piperidyl)phenoxy]pyrimidin-2-yl]-1-methyl-pyrazole-4-sulfonamide

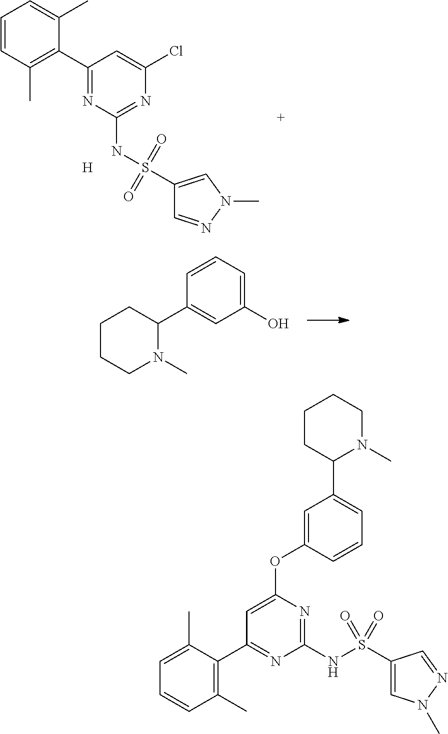

An NMP (0.5 mL) solution of N-[4-chloro-6-(2,6-dimethylphenyl)pyrimidin-2-yl]-1 -methyl-pyrazole-4-sulfonamide (20 mg, 0.05293 mmol), Cs₂CO₃ (72.1 mg, 0.2213 mmol), and 3-(1-methyl-2-piperidyl)phenol (17.6 mg, 0.0920 mmol) was stirred at 110° C. for 16 hours and then cooled to room temperature. The solution was filtered and the resulting residue diluted with 0.8 mL MeOH, and purified by reverse phase chromatography using a 15 min gradient of 20% MeCN in water to 80% MeCN (HCl modifier) to give N-[4-(2,6-dimethylphenyl)-6-[3-(1 -methyl-2-piperidyl)phenoxy]pyrimidin-2-yl]-1-methyl-pyrazole-4-sulfonamide (hydrochloride salt) (7.3 mg, 24%) ESI-MS m/z calc. 532.22565, found 533.55 (M+1)⁺; Retention time: 1.21 minutes. (LC method A).

### Example 287: Preparation of Compound 929

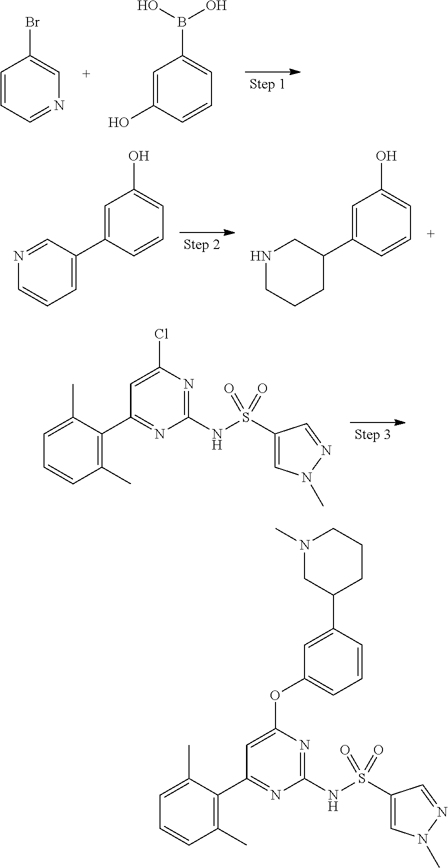

### Step 1: 3-(3-Pyridyl)phenol

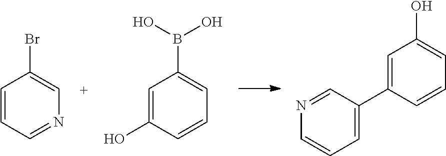 [SMILES: Brc1cccnc1 ; OB(O)c1cccc(O)c1 ; Oc1cccc(-c2cccnc2)c1]

Tetrakis(triphenylphosphine)palladium(0) (838 mg, 0.725 mmol) was added to a solution of (3-hydroxyphenyl)boronic acid (10.02 g, 72.65 mmol), 3-bromopyridine (7 mL, 72.66 mmol) and sodium carbonate (15.37 g, 145.0 mmol) in mixture of tetrahydrofuran (140.0 mL), water (70.00 mL) and methanol (35.00 mL). The reaction mixture was bubbled with nitrogen for 5 minutes and heated at reflux for 2 hours followed by stirring at room temperature overnight. Reaction mixture was diluted with water (100 mL) and extracted with EtOAc (3×100 mL). Organic layers were combined, dried over sodium sulfate and concentrated under reduced pressure. (15.39 g of orange gum) The residue was purified by silica gel chromatography using 0% to 6% of methanol in dichloromethane to afford 3-(3-pyridyl)phenol (6.31 g, 51%) as a yellow solid. ¹H NMR (300 MHz, DMSO-d₆) ppm 6.77-6.85 (m, 1H), 7.04 (s, 1H), 7.10 (d, J=7.6 Hz, 1H), 7.23-7.34 (m, 1H), 7.45 (dd, J=7.9, 5.0 Hz, 1H), 7.98 (d, J=7.9 Hz, 1H), 8.54 (d, J=4.7 Hz, 1H), 8.77-8.83 (m, 1H), 9.61 (s, 1H). ESI-MS m/z calc. 171.195, found 172.2 (M+1)⁺; Retention time: 0.7 minutes (LC method C).

### Step 2: 3-(3-Piperidyl)phenol

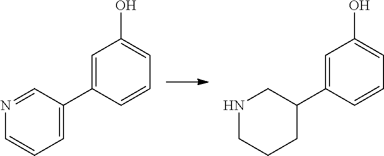 [SMILES: Oc1cccc(-c2cccnc2)c1 ; Oc1cccc(C2CCCNC2)c1]

Platinum oxide (837.0 mg, 3.686 mmol) was added to a solution of 3-(3-pyridyl)phenol (6.31 g, 36.86 mmol) in methanol (150 mL) and concentrated HCl (6 mL). Reaction mixture was placed under 50 PSI of hydrogen for 48 hours (16 hours with stirring). The reaction mixture was filtrated over Celite, washed with methanol and concentrated under reduced pressure to afford 3-(3-piperidyl)phenol (hydrochloride salt) (9.66 g, 123%) as a yellow oil. ESI-MS m/z calc. 177.243, found 178.2 (M+1)⁺; Retention time: 0.52 minutes. (LC method C).

### Step 3: N-[4-(2,6-Dimethylphenyl)-6-[3-(1-methyl-3-piperidyl)phenoxy]pyrimidin-2-yl]-1-methyl-pyrazole-4-sulfonamide

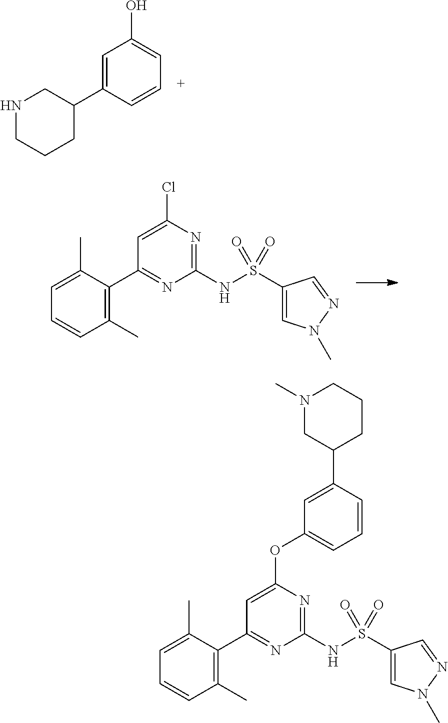

An NMP (0.8 mL) solution of 3-(3-piperidyl)phenol (51.2 mg, 0.289 mmol), Cs₂CO₃ (approximately 271 mg, 0.832 mmol) and N-[4-chloro-6-(2,6-dimethylphenyl)pyrimidin-2-yl]-1 -methyl-pyrazole-4-sulfonamide (50.7 mg, 0.1342 mmol) was heated to 110° C. for 20 hours and then cooled to room temperature. The solution was filtered and the resulting residues dissolved in 0.7 mL DMSO, and purified by reverse phase chromatography using a 15 minute gradient of 20% MeCN in water to 80% MeCN to give N-[4-(2,6-dimethylphenyl)-6-[3-(3-piperidyl)phenoxy]pyrimidin-2-yl]-1-methyl-pyrazole-4-sulfonamide (5.1 mg, 3%) ESI-MS m z calc. 518.21, found 519.55 (M+1)⁺; Retention time: 1.24 minutes and N-[4-(2,6-dimethylphenyl)-6-[3-(3-hydroxyphenyl)-1-piperidyl]pyrimidin-2-yl]-1-methyl-pyrazole-4-sulfonamide (5.6 mg, 4%) ESI-MS m/z calc. 518.21, found 519.55 (M+1)⁺; Retention time: 1.39 minutes (LC method A).

N-[4-(2,6-dimethylphenyl)-6-[3-(3-piperidyl)phenoxy]pyrimidin-2-yl]-1-methyl-pyrazole-4-sulfonamide (5.1 mg, 3%) from above was treated with formaldehyde (200 μL, 7.26 mmol) and formic acid (200 μL, 5.30 mmol) and heated to 90° C. for 18 hours and then cooled to room temperature. The solution was filtered and the resulting residues dissolved in 0.8 mL MeOH, and purified by reverse phase chromatography using a 15 minute gradient of 20% MeCN in water to 80% MeCN (HCl modifier) to give N-[4-(2,6-dimethylphenyl)-6-[3-(1 -methyl-3-piperidyl)phenoxy]pyrimidin-2-yl]-1-methyl-pyrazole-4-sulfonamide (hydrochloride salt) (2.6 mg, 2%) ESI-MS m/z calc. 532.22565, found 533.55 (M+1)⁺; Retention time: 1.24 minutes (LC method A).

### Example 288: Preparation of Compound 930

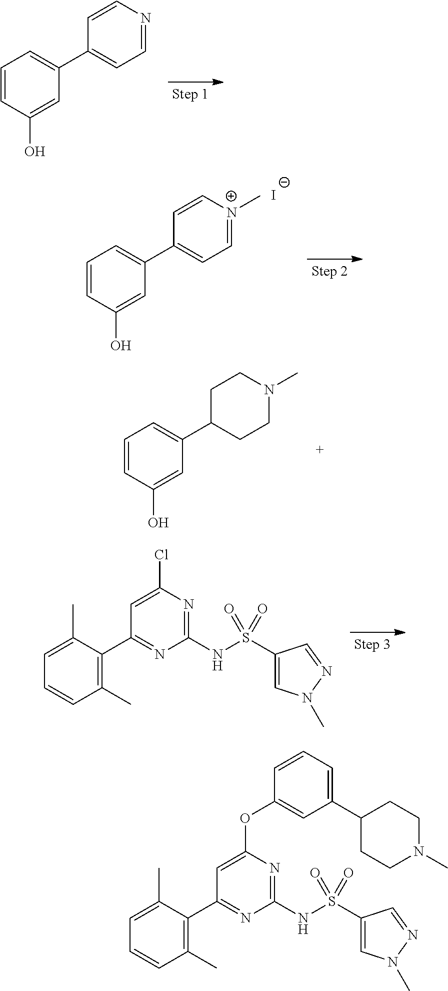

### Step 1: 3-(1-Methylpyridin-1-ium-4-yl)phenol iodide

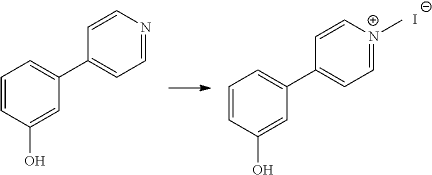

A solution of 3-pyridin-4-yl-phenol (9.27 g, 54.1 mmol) and methyl iodide (13 mL, 216.6 mmol) in acetone (550 mL) was heated at 65° C. overnight. The reaction mixture was concentrated in vacuum to half volume and solid was filtered-off, rinsed with acetone to give 4-(3-hydroxy-phenyl)-1-methyl-pyridinium iodide (16.02 g, 95%) as a yellow solid. ESI-MS m/z calc. 186.24 found 185.7 (M−1). Retention time: 1.29 minutes.

### Step 2: 3-(1-Methyl-4-piperidyl)phenol

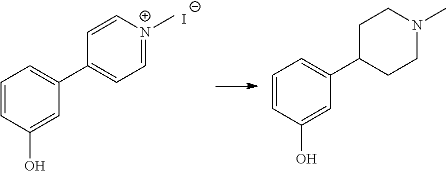

Approximately 5 g of Raney Nickel was added to 500 mL methanol, followed by the addition of 4-(3-hydroxy-phenyl)-1-methyl-pyridinium iodide (16.02 g, 51.2 mmol). The reaction was hydrogenated on a Parr shaker under 60 PSI of hydrogen for 48 hours, adding additional Raney Nickel (5 g×2) after 16 and 32 hours shaking. The reaction mixture was filtered over Celite, rinsed with methanol and concentrated under vacuum. Water (50 mL), saturated aqueous sodium bicarbonate solution (100 mL) and 10 mL brine were added to the crude and it was extracted with a mixture of 4:1 dichloromethane iso-propanol (150 mL×6). Combined organic layer was dried over anhydrous magnesium sulfate and purified by silica gel chromatography using 0-20% dichloromethane-methanol (containing 1% triethylamine). Product containing fractions were concentrated under vacuum and a 1M aqueous sodium hydroxide solution was added. The aqueous layer was extracted with ethyl acetate (100 mL×5), acidified with 2 M aqueous hydrogen chloride solution to pH 5 and then neutralized with solid sodium bicarbonate. The aqueous layer was extracted with a mixture 4:1 dichloromethane: iso-propanol (100 mL×10) to afford 3-(1-methyl-piperidin-4-yl)-phenol (5.10 g, 52%) as a white solid. ESI-MS m/z calc. 191.28 found 192.0 (M1). Retention time: 1.27 minutes. ¹H NMR (250 MHz, ACN-d₃) ppm 1.30-1.41 (m, 3H) 1.49-1.55 (m, 2H) 1.68-1.81 (m, 1H) 1.89 (s, 3H) 1.97-2.13 (m, 1H)2.50-2.68 (m, 2H) 6.17-6.36 (m, 3H) 6.70 (t, J=7.86 Hz, 1H).

### Step 3: N-[4-(2,6-Dimethylphenyl)-6-[3-(1-methyl-4-piperidyl)phenoxy]pyrimidin-2-yl]-1-methyl-pyrazole-4-sulfonamide

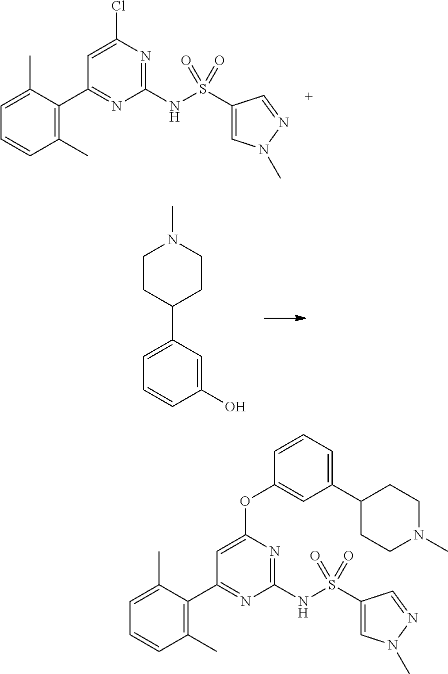 [SMILES: Cc1cccc(C)c1-c1cc(Cl)nc(NS(=O)(=O)c2cnn(C)c2)n1 ; CN1CCC(c2cccc(O)c2)CC1 ; Cc1cccc(C)c1-c1cc(Oc2cccc(C3CCN(C)CC3)c2)nc(NS(=O)(=O)c2cnn(C)c2)n1]

To a solution of N-[4-chloro-6-(2,6-dimethylphenyl)pyrimidin-2-yl]-1-methyl-pyrazole-4-sulfonamide (30 mg, 0.07940 mmol) and 3-(1-methyl-4-piperidyl)phenol (approximately 45.56 mg, 0.2382 mmol) in NMP (0.4 mL) was added Cs₂CO₃ (approximately 103.5 mg, 0.3176 mmol) and the reaction mixture was stirred at 120° C. for 16 hours. The reaction mixture was diluted with MeOH, filtered and purification by HPLC (1-99% ACN in water (HCl modifier)) gave N-[4-(2,6-dimethylphenyl)-6-[3-(1-methyl-4-piperidyl)phenoxy]pyrimidin-2-yl]-1-methyl-pyrazole-4-sulfonamide (hydrochloride salt) (27.7 mg, 58%). ESI-MS m/z calc. 532.22565, found 533.3 (M+1)⁺; Retention time: 1.03 minutes; LC method A.

### Example 289: Preparation of Compound 931

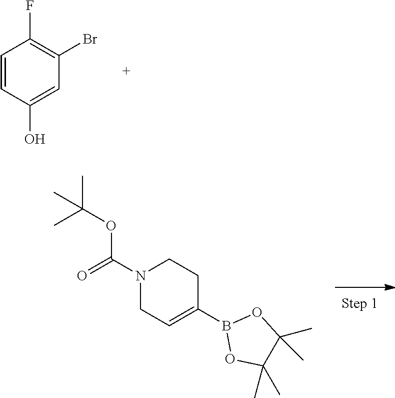 [SMILES: Oc1ccc(F)c(Br)c1 ; CC(C)(C)OC(=O)N1CC=C(B2OC(C)(C)C(C)(C)O2)CC1]

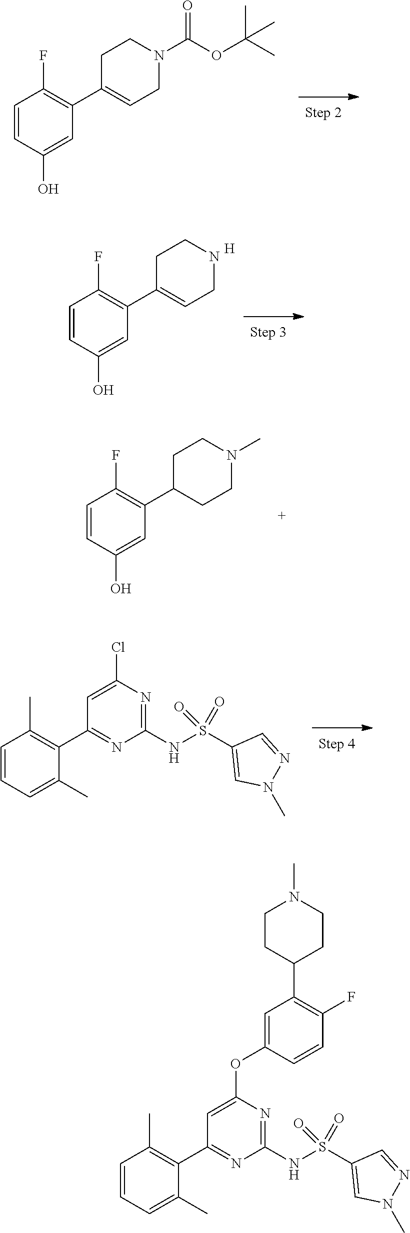

### Step 1: tert-Butyl 4-(2-fluoro-5-hydroxy-phenyl)-3,6-dihydro-2H-pyridine-1-carboxylate

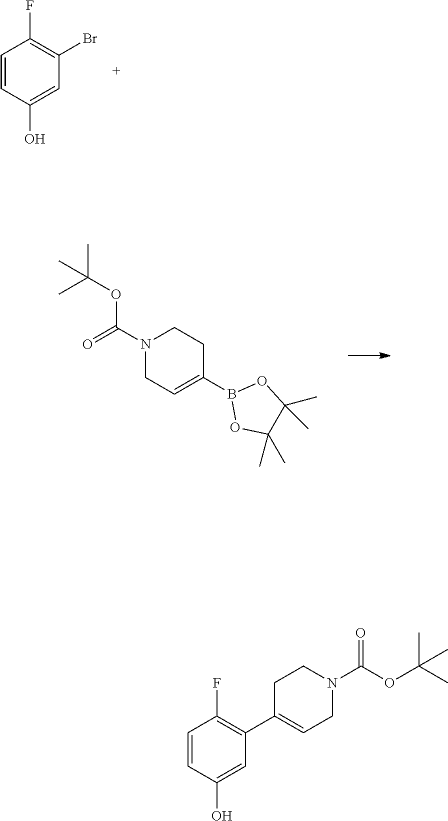 [SMILES: Oc1ccc(F)c(Br)c1 ; CC(C)(C)OC(=O)N1CC=C(B2OC(C)(C)C(C)(C)O2)CC1 ; CC(C)(C)OC(=O)N1CC=C(c2cc(O)ccc2F)CC1]

To a solution of 4-fluoro-3-bromophenol (2.37 g, 12.4 mmol) in dioxane (12 mL) and 2 M aqueous potassium carbonate solution (10 mL) were added N-tert-butoxycarbonyl-2,3,6-trahydropyridine-4-boronic acid, pinacolate (6 g, 19.4 mmol) and dichloro 1,1′-bis(diphenylphosphino)ferrocene palladium (II) dichloromethane adduct (0.4 g, 0.49 mmol). The mixture was irradiated in microwave for 25 minutes at 140° C. A total of four syntheses were made and the reaction mixture were combined, diluted with water (200 mL) and extracted with ethyl acetate (3×200 mL). The organic layer was washed with brine, dried over sodium sulfate and concentrated. The crude residue was purified by silica gel column chromatography using 0-40% hexanes-ethyl acetate to afford 4-fluoro-3-(N-tert-butyloxycarbonyl-2,3,6-trihydropyridin-4-yl)phenol (14.1 g, 97%) as an off-white solid. ESI-MS m/z: calc.293.14, found 294.5 (M+1)⁺. Retention time: 3.26 minutes.

### Step 2: 4-Fluoro-3-(1,2,3,6-tetrahydropyridin-4-yl)phenol

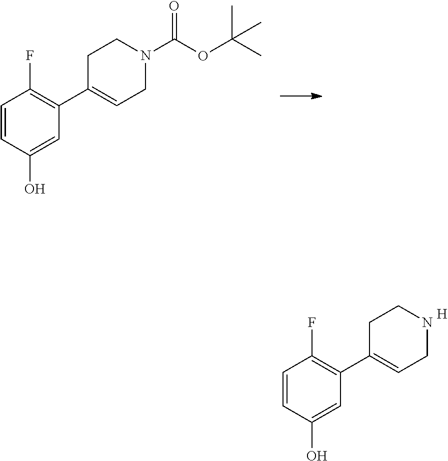

To a stirred solution of 4-fluoro-3-(N-tert-butyloxycarbonyl-2,3,6-trihydropyridin-4-yl)phenol (14.1 g, 48.10 mmol) in dichloromethane (50 mL) was added 4 N hydrogen chloride solution in dioxane (60 mL). The mixture was stirred for 1 hour at room temperature and concentrated. Diethyl ether (100 mL) was added to the residue and formed precipitate was collected by filtration to afford 4-fluoro-3-(2,3,6-trihydropyridin-4-yl)phenol hydrochloric acid salt (7.6 g, 69%) as an off-white solid. ESI-MS m/z: calc. 193.09, found 194.1 (M+1)⁺. Retention time: 1.82 minutes.

### Step 3: 4-Fluoro-3-(1-methyl-4-piperidyl)phenol

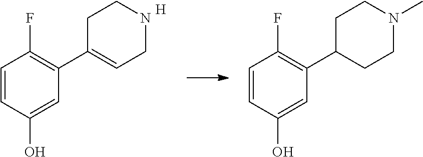

To a solution of 4-fluoro-3-(2,3,6-trihydropyridin-4-yl)phenol hydrochloric acid salt (7.6 g, 53.52 mmol) in methanol (100 mL) were added triethylamine (10 mL), a 37% aqueous formaldehyde solution (40 mL, 480 mmol) and 10% palladium on carbon (1.5 g). The mixture was stirred under hydrogen atmosphere at 50 psi for 1 hour. After filtration and concentration, the saturated sodium bicarbonate (50 mL) and ethyl acetate (200 mL) were added and extracted. The organic layer was washed with brine, dried over sodium sulfate and concentrated. The crude residue was redissolved in methanol (50 mL). 10% palladium on carbon (2.0 g) was added. The mixture was stirred under hydrogen atmosphere of 60 psi for 16 hours. The reaction mixture was filtered through Celite pad and the filtrate was concentrated to give 4-fluoro-3-(N-methylpiperidin-4-yl)phenol (4.5 g, 45%) as an off-white solid. ESI-MS m/z: calc. 209.12, found 210.2 (M+1)⁺. Retention time: 2.59 minutes. ¹H NMR (250 MHz, CDCl₃) δ (ppm): 6.55 -6.95 (m, 3H), 3.00-3.10 (m, 2H), 2.75-2.90 (m, 1H), 2.34 (s, 3H), 2.015-2.25 (m, 2H), 1.60-1.80 (m, 4H).

### Step 4: N-[4-(2,6-Dimethylphenyl)-6-[4-fluoro-3-(1-methyl-4-piperidyl)phenoxy]pyrimidin-2-yl]-1-methyl-pyrazole-4-sulfonamide

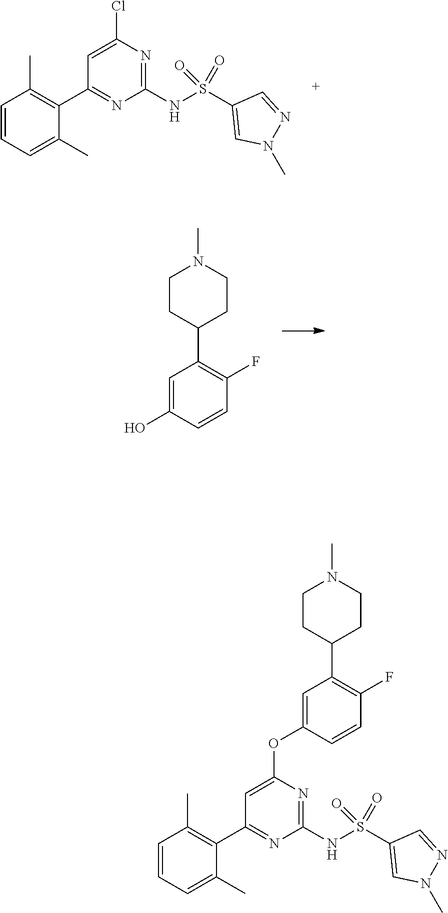 [SMILES: Cc1cccc(C)c1-c1cc(Cl)nc(NS(=O)(=O)c2cnn(C)c2)n1 ; CN1CCC(c2cc(O)ccc2F)CC1 ; Cc1cccc(C)c1-c1cc(Oc2ccc(F)c(C3CCN(C)CC3)c2)nc(NS(=O)(=O)c2cnn(C)c2)n1]

To a mixture of N-[4-chloro-6-(2,6-dimethylphenyl)pyrimidin-2-yl]-1-methyl-pyrazole-4-sulfonamide (37.78 mg, 0.1 mmol) and Cs₂CO₃ (approximately 130.3 mg, 0.4000 mmol) in NMP (0.4 mL) was added 4-fluoro-3-(1-methyl-4-piperidyl)phenol (approximately 62.78 mg, 0.3000 mmol) and the reaction mixture stirred at 120° C. for 16 hours. The reaction mixture was diluted with MeOH, filtered and purification by HPLC (1-99% ACN in water (HCl modifier)) gave N-[4-(2,6-dimethylphenyl)-6-[4-fluoro-3-(1-methyl-4-piperidyl)phenoxy]pyrimidin-2-yl]-1-methyl-pyrazole-4-sulfonamide (hydrochloride salt) (31.7 mg, 58%). ESI-MS m/z calc. 550.21625, found 551.3 (M+1)⁺; Retention time: 1.12 minutes; LC method A.

### Example 290: Preparation of Compound 932

### Step 1: tert-Butyl 4-(4-fluoro-3-hydroxy-phenyl)-3,6-dihydro-2H-pyridine-1-carboxylate

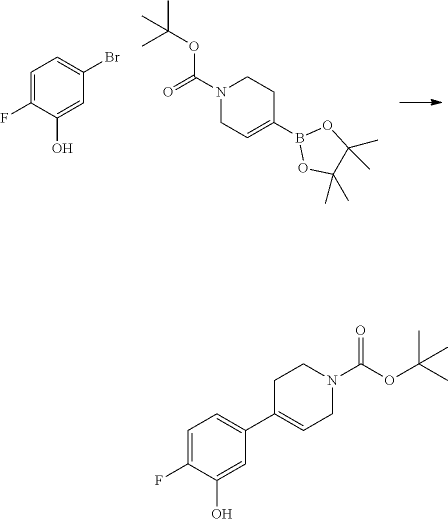

To a solution of 2-fluoro-5-bromophenol (2.25 g, 11.8 mmol) in dioxane (12 mL) and a 2 M aqueous potassium carbonate solution (10 mL) were added N-tert-butoxycarbonyl-2,3,6-trahydropyridine-4-boronic acid, pinacolate (4.5 g, 14.5 mmol) and dichloro 1,1′-bis(diphenylphosphino)ferrocene palladium (II) (dichloromethane adduct, 0.30 g, 0.37 mmol) and the mixture was heated in a microwave oven for 25 minutes at 140° C. A total of 4 syntheses were made and the reaction mixtures were combined, diluted with water (200 mL) and extracted with ethyl acetate (3×200 mL). The organic layer was washed with brine, dried over sodium sulfate and concentrated. The crude residue was purified by silica gel column chromatography using 0-40% hexane-ethyl acetate to afford 2-fluoro-5-(N-tert-butyloxycarbonyl-2,3,6-trihydropyridin-4-yl)phenol (12.70 g, 92%) as an off-white solid. ESI-MS m/z: calc.293.14, found 294.2 (M+1)⁺. Retention time: 3.33 minutes.

### Step 2: tert-Butyl 4-(4-fluoro-3-hydroxy-phenyl)piperidine-1-carboxylate

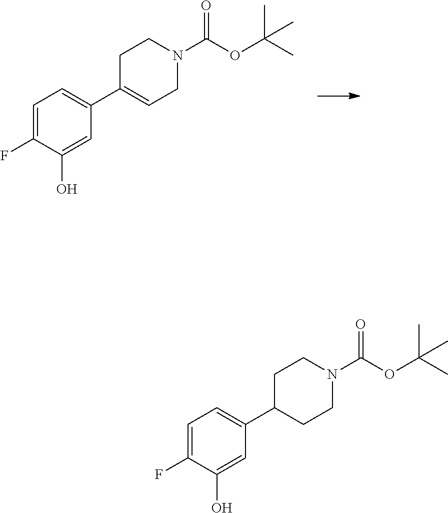 [SMILES: CC(C)(C)OC(=O)N1CC=C(c2ccc(F)c(O)c2)CC1 ; CC(C)(C)OC(=O)N1CCC(c2ccc(F)c(O)c2)CC1]

To a solution of 2-fluoro-5-(N-tert-butyloxycarbonyl-2,3,6-trihydropyridin-4-yl)phenol (12.7 g, 43.34 mmol) in methanol (80 mL) were added 10% palladium on carbon (1.2 g) and the mixture was stirred under hydrogen atmosphere at 60 psi for 2 hours. The reaction mixture was filtered through a Celite pad and the filtrate was concentrated to give 2-fluoro-5-(N-tert-butoxycarbonyl-piperidin-4-yl)phenol (13.43 g, >100%) as an off-white solid. ESI-MS m/z: calc. 295.16, found 296.2 (M+1)⁺, Retention time: 3.52 minutes.

### Step 3: 2-Fluoro-5-(4-piperidyl)phenol

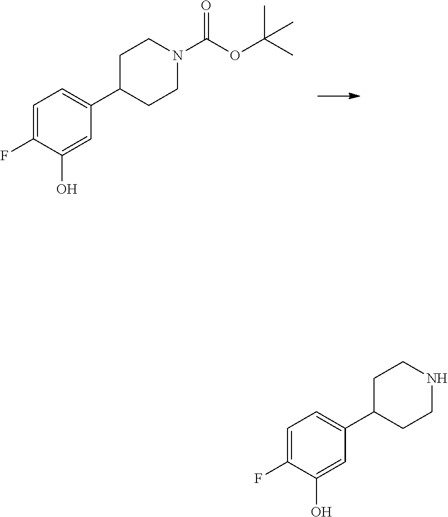 [SMILES: CC(C)(C)OC(=O)N1CCC(c2ccc(F)c(O)c2)CC1 ; Oc1cc(C2CCNCC2)ccc1F]

To a solution of 2-fluoro-5-(N-tert-butoxycarbonyl-piperidin-4-yl)phenol (13.43 g, 43.34 mmol) in dichloromethane (50 mL) was added 4 N hydrogen chloride solution in dioxane (60 mL). The mixture was stirred for 1 hour at room temperature and concentrated. Diethyl ether (100 mL) was added to the residue and formed precipitate was collected by filtration to afford 2-fluoro-5-(piperidin-4-yl)phenol hydrochloric acid salt (9.80 g, 97%) as an off-white solid. ESI-MS m/z: calc. 195.11, found 196.3 (M+1)⁺. Retention time: 1.88 minutes.

### Step 4: 2-Fluoro-5-(1-methyl-4-piperidyl)phenol

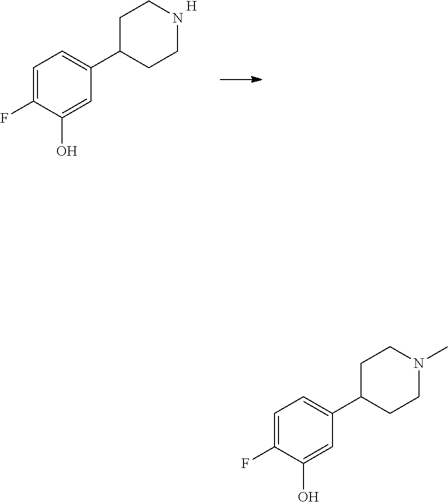

To a solution of 2-fluoro-5-(piperidin-4-yl)phenol hydrochloric acid salt (9.80 g, 42.31 mmol) in methanol (100 mL), triethylamine (10 mL) and 37% aqueous formaldehyde solution (40 mL, 480 mmol) were added 10% palladium on carbon (1.5 g, 1.41 mmol). The mixture was stirred under hydrogen atmosphere at 50 psi for 1 hour, filtered and concentrated. A saturated aqueous sodium bicarbonate solution (70 mL) and ethyl acetate (300 mL) were added The organic layer was washed with brine, dried over sodium sulfate and concentrated to give 2-fluoro-5-(N-methylpiperidin-4-yl)phenol(4.60 g, 52%) as an off-white solid. ESI-MS m/z: calc. 209.12, found 210.2 (M+1)⁺. Retention time: 2.72 minutes. ¹H NMR (250 MHz, CDCl₃) δ (ppm): 6.50-7.00 (m, 3H), 2.95-3.10 (m, 2H), 2.20-2.40 (m, 1H), 2.34 (s, 3H), 2.05-2.15 (m, 2H), 1.65-1.80 (m, 4H).

### Step 5: N-[4-(2,6-Dimethylphenyl)-6-[2-fluoro-5-(1-methyl-4-piperidyl)phenoxy]pyrimidin-2-yl]-1-methyl-pyrazole-4-sulfonamide

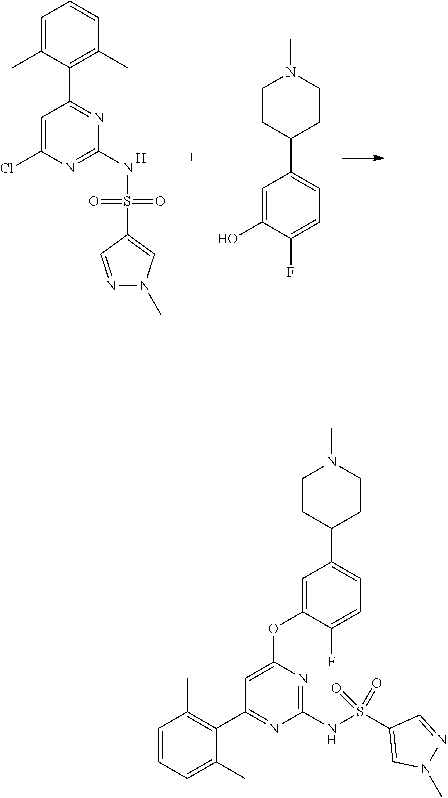

To a mixture of N-[4-chloro-6-(2,6-dimethylphenyl)pyrimidin-2-yl]-1-methyl-pyrazole-4-sulfonamide (37.78 mg, 0.1 mmol) and Cs₂CO₃ (approximately 130.3 mg, 0.4000 mmol) in NMP (0.4 mL) was added 2-fluoro-5-(1-methyl-4-piperidyl)phenol (approximately 62.78 mg, 0.3000 mmol) and the reaction mixture stirred at 120° C. for 16 hours. The reaction mixture was diluted with MeOH, filtered and purification by HPLC (1-99% ACN in water (HCl modifier)) gave N-[4-(2,6-dimethylphenyl)-6-[2-fluoro-5-(1-methyl-4-piperidyl)phenoxy]pyrimidin-2-yl]-1-methyl-pyrazole-4-sulfonamide (hydrochloride salt) (31.6 mg, 57%). ESI-MS m/z calc. 550.21625, found 551.3 (M+1)⁺; Retention time: 1.16 minutes; LC method A.

### Example 291: Characterization of Compounds 933-1068

The compounds in the following tables were prepared in a manner analogous to that described above using commercially available reagents and intermediates described herein.

| | | LCMS Room | | | |
|---|---|---|---|---|---|
| Compound | | Temperature | Calc. | | LCMS |
| Number | Structure | (min) | mass | M + 1 | Method |
| 933 | 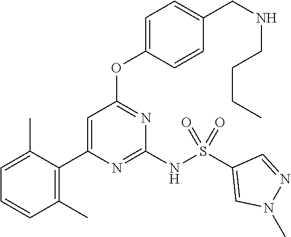 [SMILES: CCCCNCc1ccc(Oc2cc(-c3c(C)cccc3C)nc(NS(=O)(=O)c3cnn(C)c3)n2)cc1] | 1.5 | 520.226 | 521.58 | A |
| 934 | 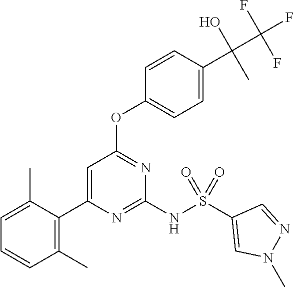 [SMILES: Cc1cccc(C)c1-c1cc(Oc2ccc(C(C)(O)C(F)(F)F)cc2)nc(NS(=O)(=O)c2cnn(C)c2)n1] | 1.73 | 547.15 | 548.47 | A |
| 935 | 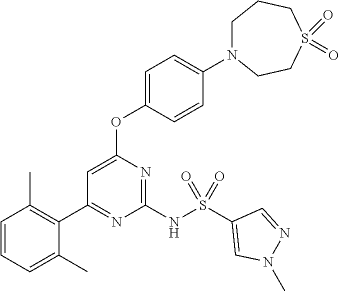 [SMILES: Cc1cccc(C)c1-c1cc(Oc2ccc(N3CCCS(=O)(=O)CC3)cc2)nc(NS(=O)(=O)c2cnn(C)c2)n1] | 1.1 | 582.172 | 583.41 | A |
| 936 | 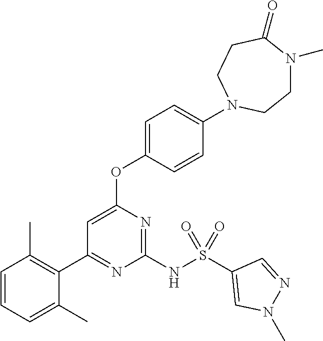 [SMILES: Cc1cccc(C)c1-c1cc(Oc2ccc(N3CCC(=O)N(C)CC3)cc2)nc(NS(=O)(=O)c2cnn(C)c2)n1] | 1.44 | 561.216 | 562.62 | A |
| 937 | 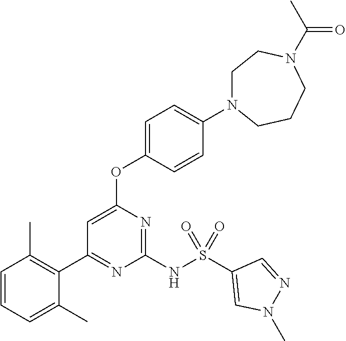 [SMILES: CC(=O)N1CCCN(c2ccc(Oc3cc(-c4c(C)cccc4C)nc(NS(=O)(=O)c4cnn(C)c4)n3)cc2)CC1] | 1.08 | 575.231 | 576.44 | A |
| 938 | 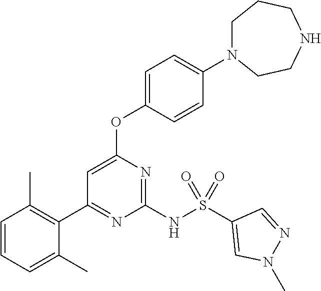 [SMILES: Cc1cccc(C)c1-c1cc(Oc2ccc(N3CCCNCC3)cc2)nc(NS(=O)(=O)c2cnn(C)c2)n1] | 0.86 | 533.221 | 534.44 | A |
| 939 | 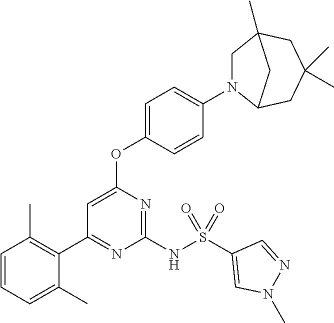 [SMILES: Cc1cccc(C)c1-c1cc(Oc2ccc(N3CC4(C)CC3CC(C)(C)C4)cc2)nc(NS(=O)(=O)c2cnn(C)c2)n1] | 2.46 | 586.273 | 587.7 | A |
| 940 | 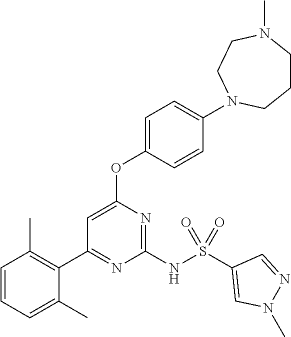 [SMILES: Cc1cccc(C)c1-c1cc(Oc2ccc(N3CCCN(C)CC3)cc2)nc(NS(=O)(=O)c2cnn(C)c2)n1] | 1.24 | 547.237 | 548.65 | A |
| 941 | 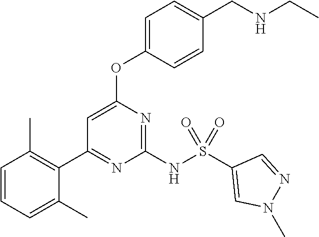 [SMILES: CCNCc1ccc(Oc2cc(-c3c(C)cccc3C)nc(NS(=O)(=O)c3cnn(C)c3)n2)cc1] | 1.15 | 492.194 | 493.6 | A |
| 942 | 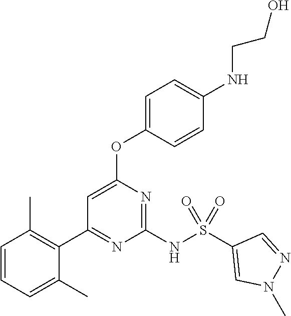 [SMILES: Cc1cccc(C)c1-c1cc(Oc2ccc(NCCO)cc2)nc(NS(=O)(=O)c2cnn(C)c2)n1] | 1.2 | 494.174 | 495.49 | A |
| 943 | 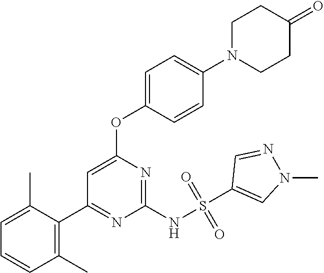 [SMILES: Cc1cccc(C)c1-c1cc(Oc2ccc(N3CCC(=O)CC3)cc2)nc(NS(=O)(=O)c2cnn(C)c2)n1] | 1.52 | 532.189 | 533.48 | A |
| 944 | 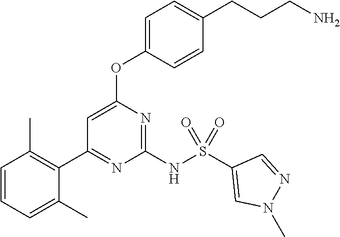 [SMILES: Cc1cccc(C)c1-c1cc(Oc2ccc(CCCN)cc2)nc(NS(=O)(=O)c2cnn(C)c2)n1] | 1.21 | 492.194 | 493.46 | A |
| 945 | 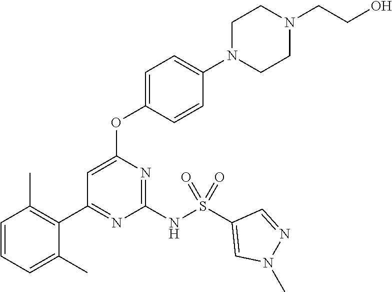 [SMILES: Cc1cccc(C)c1-c1cc(Oc2ccc(N3CCN(CCO)CC3)cc2)nc(NS(=O)(=O)c2cnn(C)c2)n1] | 1.17 | 563.231 | 564.54 | A |
| 946 | 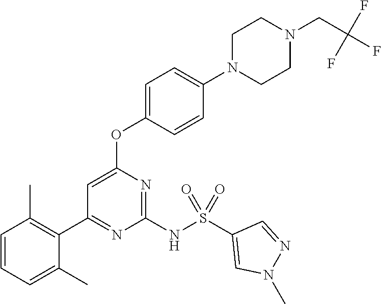 [SMILES: Cc1cccc(C)c1-c1cc(Oc2ccc(N3CCN(CC(F)(F)F)CC3)cc2)nc(NS(=O)(=O)c2cnn(C)c2)n1] | 1.75 | 601.208 | 602.5 | A |
| 947 | 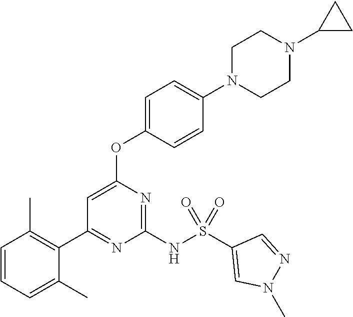 [SMILES: Cc1cccc(C)c1-c1cc(Oc2ccc(N3CCN(C4CC4)CC3)cc2)nc(NS(=O)(=O)c2cnn(C)c2)n1] | 1.245 | 559.237 | 560.55 | A |
| 948 | 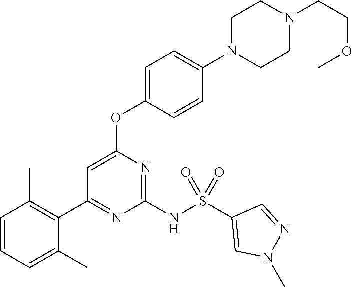 [SMILES: COCCN1CCN(c2ccc(Oc3cc(-c4c(C)cccc4C)nc(NS(=O)(=O)c4cnn(C)c4)n3)cc2)CC1] | 1.26 | 577.247 | 578.51 | A |
| 949 | 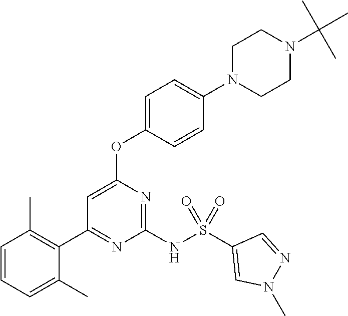 [SMILES: Cc1cccc(C)c1-c1cc(Oc2ccc(N3CCN(C(C)(C)C)CC3)cc2)nc(NS(=O)(=O)c2cnn(C)c2)n1] | 1.29 | 575.268 | 576.55 | A |
| 950 | 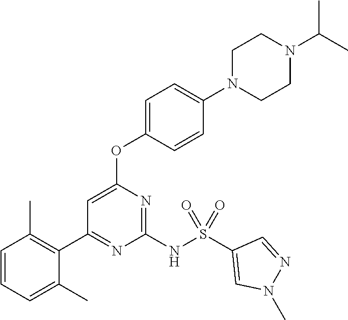 [SMILES: Cc1cccc(C)c1-c1cc(Oc2ccc(N3CCN(C(C)C)CC3)cc2)nc(NS(=O)(=O)c2cnn(C)c2)n1] | 1.26 | 561.252 | 562.55 | A |
| 951 | 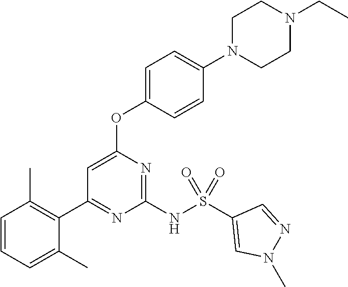 [SMILES: CCN1CCN(c2ccc(Oc3cc(-c4c(C)cccc4C)nc(NS(=O)(=O)c4cnn(C)c4)n3)cc2)CC1] | 1.23 | 547.237 | 548.54 | A |
| 952 | 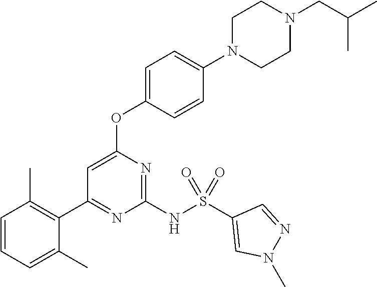 [SMILES: Cc1cccc(C)c1-c1cc(Oc2ccc(N3CCN(CC(C)C)CC3)cc2)nc(NS(=O)(=O)c2cnn(C)c2)n1] | 1.32 | 575.268 | 576.55 | A |
| 953 | 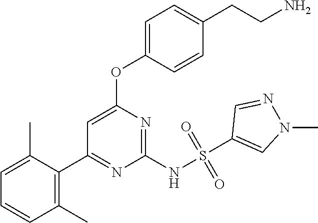 [SMILES: Cc1cccc(C)c1-c1cc(Oc2ccc(CCN)cc2)nc(NS(=O)(=O)c2cnn(C)c2)n1] | 1.14 | 478.179 | 474.49 | A |
| 954 | 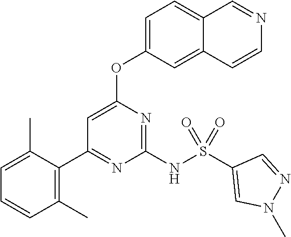 [SMILES: Cc1cccc(C)c1-c1cc(Oc2ccc3cnccc3c2)nc(NS(=O)(=O)c2cnn(C)c2)n1] | 1.06 | 486.147 | 487.41 | A |
| 955 | 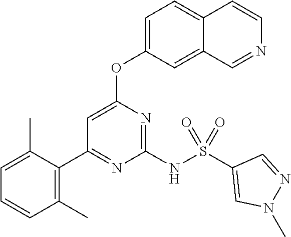 [SMILES: Cc1cccc(C)c1-c1cc(Oc2ccc3ccncc3c2)nc(NS(=O)(=O)c2cnn(C)c2)n1] | 1.06 | 486.147 | 487.44 | A |
| 956 | 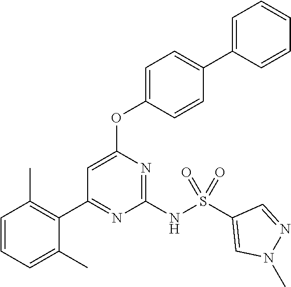 [SMILES: Cc1cccc(C)c1-c1cc(Oc2ccc(-c3ccccc3)cc2)nc(NS(=O)(=O)c2cnn(C)c2)n1] | 1.94 | 511.168 | 512.48 | A |
| 957 | 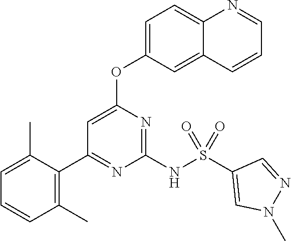 [SMILES: Cc1cccc(C)c1-c1cc(Oc2ccc3ncccc3c2)nc(NS(=O)(=O)c2cnn(C)c2)n1] | 1.16 | 486.147 | 487.41 | A |
| 958 | 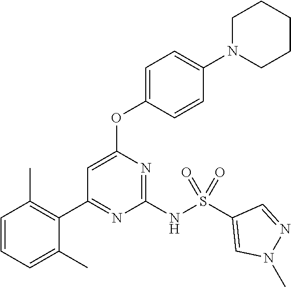 [SMILES: Cc1cccc(C)c1-c1cc(Oc2ccc(N3CCCCC3)cc2)nc(NS(=O)(=O)c2cnn(C)c2)n1] | 1.23 | 518.21 | 519.5 | A |
| 959 | 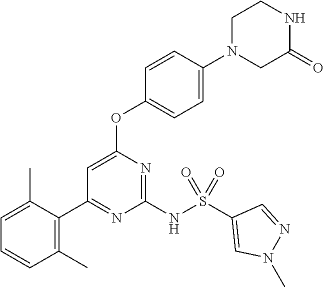 [SMILES: Cc1cccc(C)c1-c1cc(Oc2ccc(N3CCNC(=O)C3)cc2)nc(NS(=O)(=O)c2cnn(C)c2)n1] | 1.32 | 533.185 | 534.43 | A |
| 960 | 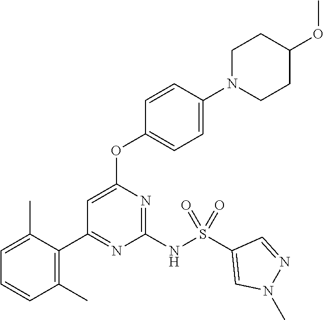 [SMILES: COC1CCN(c2ccc(Oc3cc(-c4c(C)cccc4C)nc(NS(=O)(=O)c4cnn(C)c4)n3)cc2)CC1] | 1.23 | 548.221 | 549.52 | A |
| 961 | 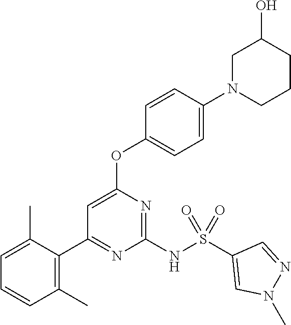 [SMILES: Cc1cccc(C)c1-c1cc(Oc2ccc(N3CCCC(O)C3)cc2)nc(NS(=O)(=O)c2cnn(C)c2)n1] | 1.17 | 534.205 | 535.48 | A |
| 962 | 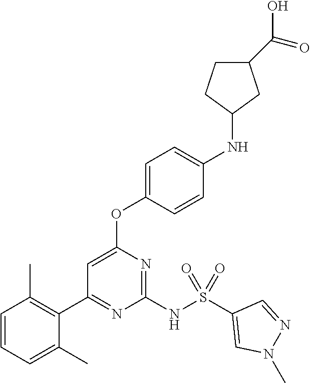 [SMILES: Cc1cccc(C)c1-c1cc(Oc2ccc(NC3CCC(C(=O)O)C3)cc2)nc(NS(=O)(=O)c2cnn(C)c2)n1] | 1.26 | 562.2 | 563.49 | A |
| 963 | 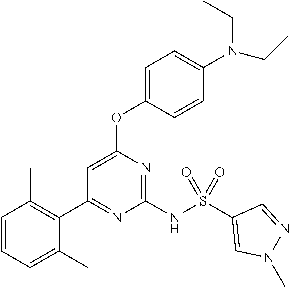 [SMILES: CCN(CC)c1ccc(Oc2cc(-c3c(C)cccc3C)nc(NS(=O)(=O)c3cnn(C)c3)n2)cc1] | 1.12 | 506.21 | 507.5 | A |
| 964 | 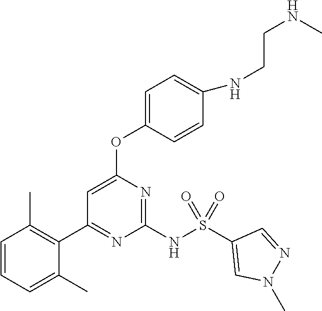 [SMILES: CNCCNc1ccc(Oc2cc(-c3c(C)cccc3C)nc(NS(=O)(=O)c3cnn(C)c3)n2)cc1] | 1.06 | 507.205 | 508.52 | A |
| 965 | 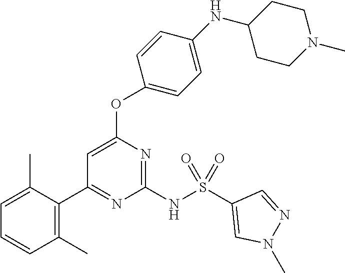 [SMILES: Cc1cccc(C)c1-c1cc(Oc2ccc(NC3CCN(C)CC3)cc2)nc(NS(=O)(=O)c2cnn(C)c2)n1] | 1.09 | 547.237 | 548.54 | A |
| 966 | 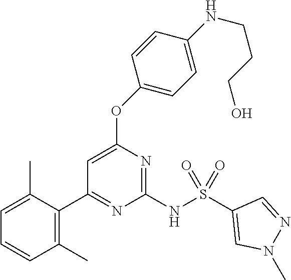 [SMILES: Cc1cccc(C)c1-c1cc(Oc2ccc(NCCCO)cc2)nc(NS(=O)(=O)c2cnn(C)c2)n1] | 1.1 | 508.189 | 508.48 | A |
| 967 | 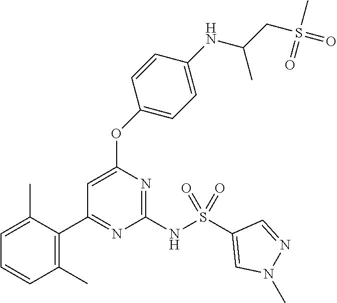 [SMILES: Cc1cccc(C)c1-c1cc(Oc2ccc(NC(C)CS(C)(=O)=O)cc2)nc(NS(=O)(=O)c2cnn(C)c2)n1] | 1.465 | 570.172 | 571.2 | A |
| 968 | 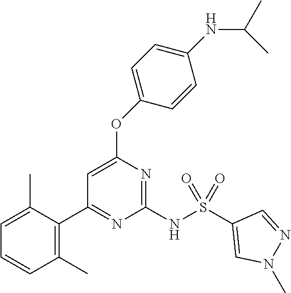 [SMILES: Cc1cccc(C)c1-c1cc(Oc2ccc(NC(C)C)cc2)nc(NS(=O)(=O)c2cnn(C)c2)n1] | 1.28 | 492.194 | 492.96 | A |
| 969 | 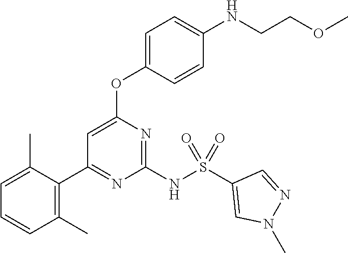 [SMILES: COCCNc1ccc(Oc2cc(-c3c(C)cccc3C)nc(NS(=O)(=O)c3cnn(C)c3)n2)cc1] | 1.41 | 508.189 | 508.95 | A |
| 970 | 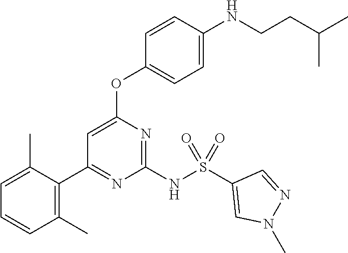 [SMILES: Cc1cccc(C)c1-c1cc(Oc2ccc(NCCC(C)C)cc2)nc(NS(=O)(=O)c2cnn(C)c2)n1] | 1.5 | 520.226 | 521.5 | A |
| 971 | 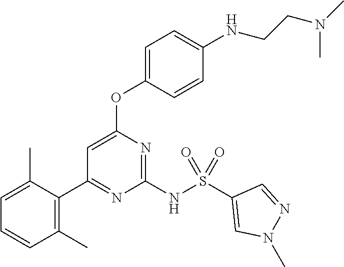 [SMILES: Cc1cccc(C)c1-c1cc(Oc2ccc(NCCN(C)C)cc2)nc(NS(=O)(=O)c2cnn(C)c2)n1] | 1.07 | 521.221 | 522.53 | A |
| 972 | 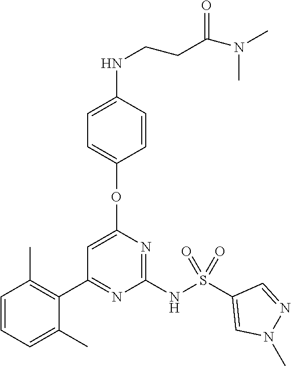 [SMILES: Cc1cccc(C)c1-c1cc(Oc2ccc(NCCC(=O)N(C)C)cc2)nc(NS(=O)(=O)c2cnn(C)c2)n1] | 1.21 | 549.216 | 550.5 | A |
| 973 | 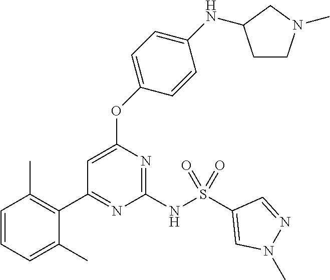 [SMILES: Cc1cccc(C)c1-c1cc(Oc2ccc(NC3CCN(C)C3)cc2)nc(NS(=O)(=O)c2cnn(C)c2)n1] | 1.08 | 533.221 | 534.54 | A |
| 974 | 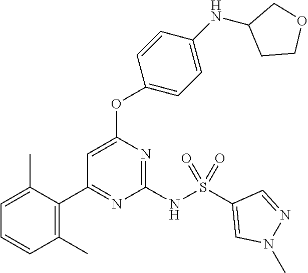 [SMILES: Cc1cccc(C)c1-c1cc(Oc2ccc(NC3CCOC3)cc2)nc(NS(=O)(=O)c2cnn(C)c2)n1] | 1.47 | 520.189 | 520.94 | A |
| 975 | 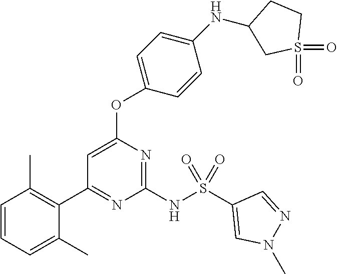 [SMILES: Cc1cccc(C)c1-c1cc(Oc2ccc(NC3CCS(=O)(=O)C3)cc2)nc(NS(=O)(=O)c2cnn(C)c2)n1] | 1.46 | 568.156 | 569.06 | A |
| 976 | 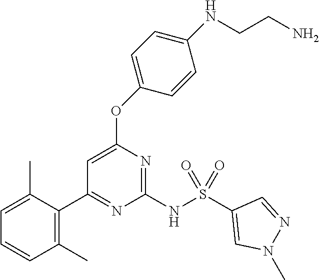 [SMILES: Cc1cccc(C)c1-c1cc(Oc2ccc(NCCN)cc2)nc(NS(=O)(=O)c2cnn(C)c2)n1] | 1.05 | 493.19 | 494.48 | A |
| 977 | 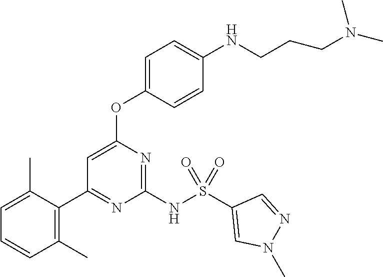 [SMILES: Cc1cccc(C)c1-c1cc(Oc2ccc(NCCCN(C)C)cc2)nc(NS(=O)(=O)c2cnn(C)c2)n1] | 1.08 | 535.237 | 536.53 | A |
| 978 | 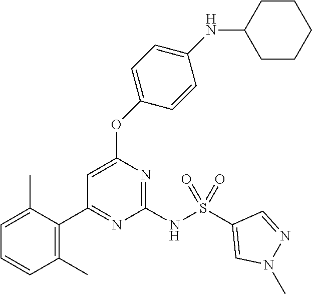 [SMILES: Cc1cccc(C)c1-c1cc(Oc2ccc(NC3CCCCC3)cc2)nc(NS(=O)(=O)c2cnn(C)c2)n1] | 1.43 | 532.226 | 533.2 | A |
| 979 | 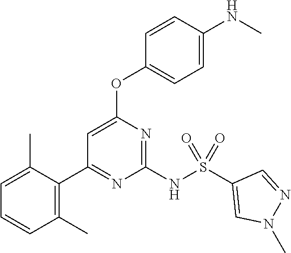 [SMILES: CNc1ccc(Oc2cc(-c3c(C)cccc3C)nc(NS(=O)(=O)c3cnn(C)c3)n2)cc1] | 1.26 | 464.163 | 465.1 | A |
| 980 | 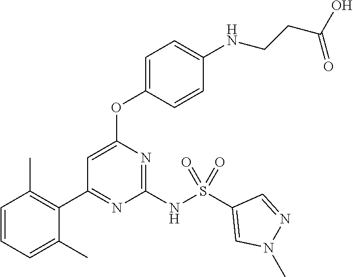 [SMILES: Cc1cccc(C)c1-c1cc(Oc2ccc(NCCC(=O)O)cc2)nc(NS(=O)(=O)c2cnn(C)c2)n1] | 1.26 | 522.169 | 523.47 | A |
| 981 | 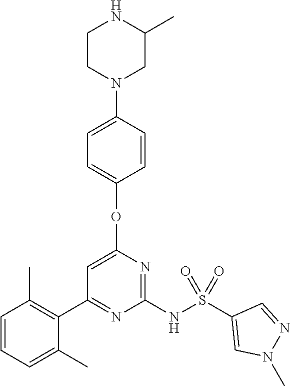 [SMILES: Cc1cccc(C)c1-c1cc(Oc2ccc(N3CCNC(C)C3)cc2)nc(NS(=O)(=O)c2cnn(C)c2)n1] | 1.12 | 533.221 | 534.54 | A |
| 982 | 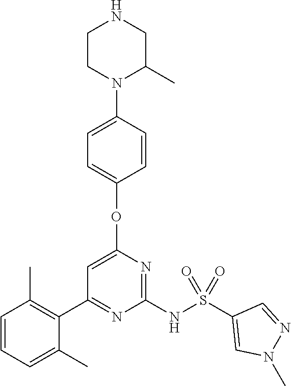 [SMILES: Cc1cccc(C)c1-c1cc(Oc2ccc(N3CCNCC3C)cc2)nc(NS(=O)(=O)c2cnn(C)c2)n1] | 1.12 | 533.221 | 534.54 | A |
| 983 | 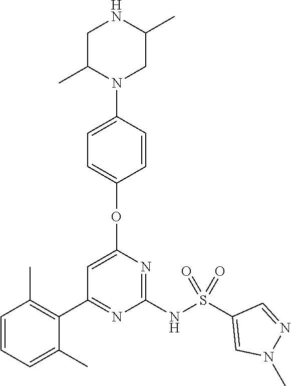 [SMILES: Cc1cccc(C)c1-c1cc(Oc2ccc(N3CC(C)NCC3C)cc2)nc(NS(=O)(=O)c2cnn(C)c2)n1] | 1.16 | 547.237 | 548.54 | A |
| 984 | 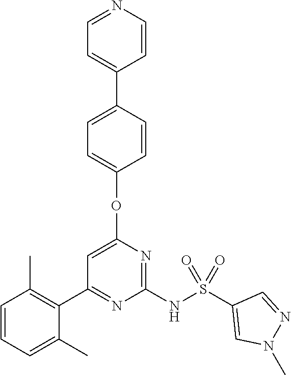 [SMILES: Cc1cccc(C)c1-c1cc(Oc2ccc(-c3ccncc3)cc2)nc(NS(=O)(=O)c2cnn(C)c2)n1] | 1.12 | 512.163 | 513.46 | A |
| 985 | 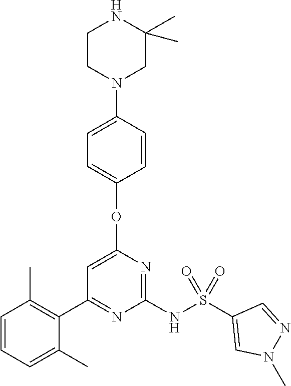 [SMILES: Cc1cccc(C)c1-c1cc(Oc2ccc(N3CCNC(C)(C)C3)cc2)nc(NS(=O)(=O)c2cnn(C)c2)n1] | 1.18 | 547.237 | 548.54 | A |
| 986 | 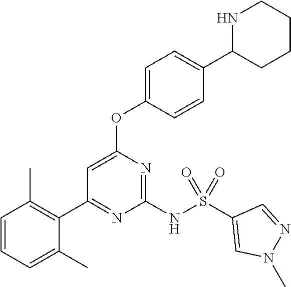 [SMILES: Cc1cccc(C)c1-c1cc(Oc2ccc(C3CCCCN3)cc2)nc(NS(=O)(=O)c2cnn(C)c2)n1] | 1.11 | 518.21 | 519.51 | A |
| 987 | 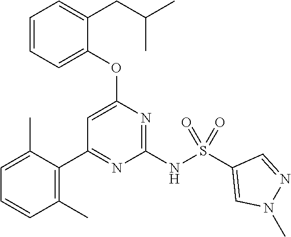 [SMILES: Cc1cccc(C)c1-c1cc(Oc2ccccc2CC(C)C)nc(NS(=O)(=O)c2cnn(C)c2)n1] | 2.08 | 491.199 | 492.3 | A |
| 988 | 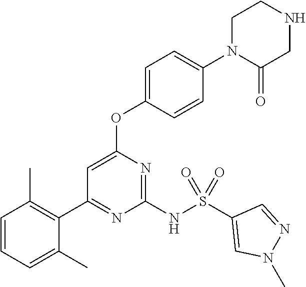 [SMILES: Cc1cccc(C)c1-c1cc(Oc2ccc(N3CCNCC3=O)cc2)nc(NS(=O)(=O)c2cnn(C)c2)n1] | 1.25 | 533.185 | 534.5 | A |
| 989 | 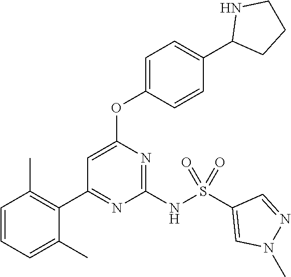 [SMILES: Cc1cccc(C)c1-c1cc(Oc2ccc(C3CCCN3)cc2)nc(NS(=O)(=O)c2cnn(C)c2)n1] | 1.28 | 504.194 | 505.51 | A |
| 990 | 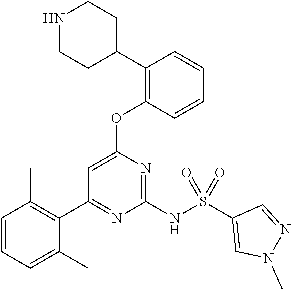 [SMILES: Cc1cccc(C)c1-c1cc(Oc2ccccc2C2CCNCC2)nc(NS(=O)(=O)c2cnn(C)c2)n1] | 1.11 | 518.21 | 519.51 | A |
| 991 | 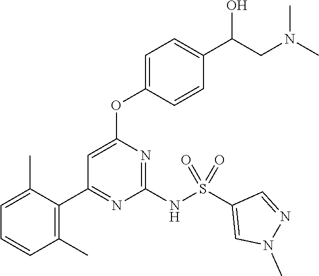 [SMILES: Cc1cccc(C)c1-c1cc(Oc2ccc(C(O)CN(C)C)cc2)nc(NS(=O)(=O)c2cnn(C)c2)n1] | 1.11 | 522.205 | 523.54 | A |
| 992 | 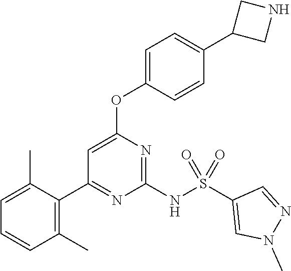 [SMILES: Cc1cccc(C)c1-c1cc(Oc2ccc(C3CNC3)cc2)nc(NS(=O)(=O)c2cnn(C)c2)n1] | 1.12 | 490.179 | 491.5 | A |
| 993 | 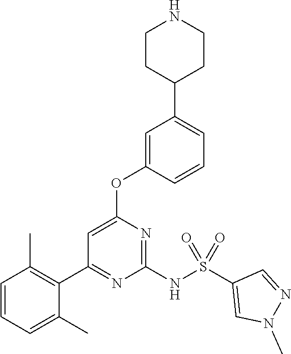 [SMILES: Cc1cccc(C)c1-c1cc(Oc2cccc(C3CCNCC3)c2)nc(NS(=O)(=O)c2cnn(C)c2)n1] | 1.18 | 518.21 | 519.51 | A |
| 994 | 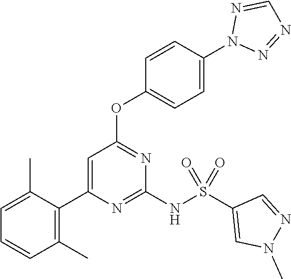 [SMILES: Cc1cccc(C)c1-c1cc(Oc2ccc(-n3ncnn3)cc2)nc(NS(=O)(=O)c2cnn(C)c2)n1] | 1.49 | 503.149 | 504.49 | A |
| 995 | 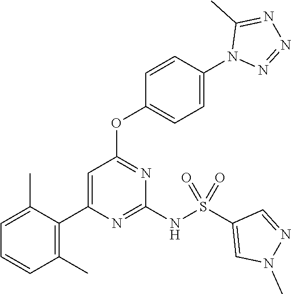 [SMILES: Cc1cccc(C)c1-c1cc(Oc2ccc(-n3nnnc3C)cc2)nc(NS(=O)(=O)c2cnn(C)c2)n1] | 1.5 | 517.164 | 518.5 | A |
| 996 | 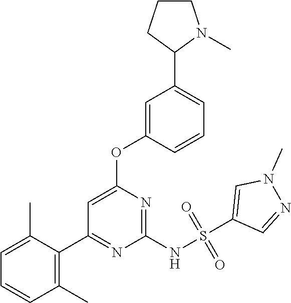 [SMILES: Cc1cccc(C)c1-c1cc(Oc2cccc(C3CCCN3C)c2)nc(NS(=O)(=O)c2cnn(C)c2)n1] | 1.23 | 518.21 | 519.55 | A |
| 997 | 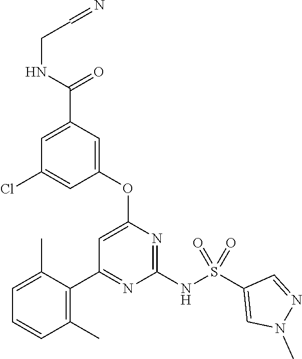 [SMILES: Cc1cccc(C)c1-c1cc(Oc2cc(Cl)cc(C(=O)NCC=N)c2)nc(NS(=O)(=O)c2cnn(C)c2)n1] | 1.63 | 551.114 | 552.18 | A |
| 998 | 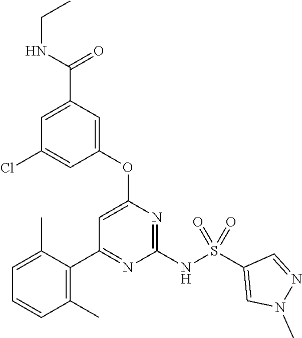 [SMILES: CCNC(=O)c1cc(Cl)cc(Oc2cc(-c3c(C)cccc3C)nc(NS(=O)(=O)c3cnn(C)c3)n2)c1] | 1.65 | 540.135 | 540.59 | A |
| 999 | 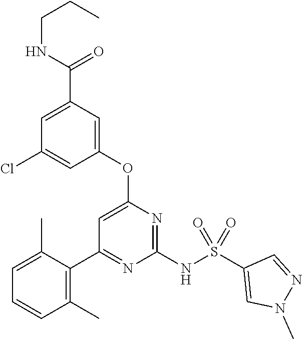 [SMILES: CCCNC(=O)c1cc(Cl)cc(Oc2cc(-c3c(C)cccc3C)nc(NS(=O)(=O)c3cnn(C)c3)n2)c1] | 1.75 | 554.15 | 555.15 | A |
| 1000 | 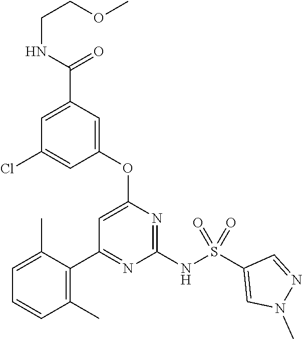 [SMILES: COCCNC(=O)c1cc(Cl)cc(Oc2cc(-c3c(C)cccc3C)nc(NS(=O)(=O)c3cnn(C)c3)n2)c1] | 1.62 | 570.145 | 571.19 | A |
| 1001 | 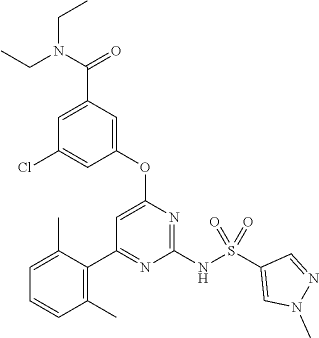 [SMILES: CCN(CC)C(=O)c1cc(Cl)cc(Oc2cc(-c3c(C)cccc3C)nc(NS(=O)(=O)c3cnn(C)c3)n2)c1] | 1.8 | 568.166 | 569.16 | A |
| 1002 | 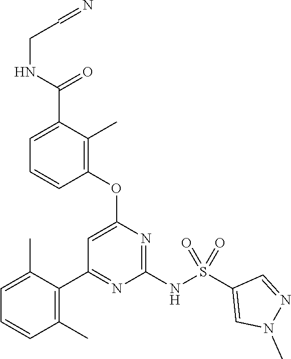 [SMILES: Cc1cccc(C)c1-c1cc(Oc2cccc(C(=O)NCC=N)c2C)nc(NS(=O)(=O)c2cnn(C)c2)n1] | 1.5 | 531.169 | 532.01 | A |
| 1003 | 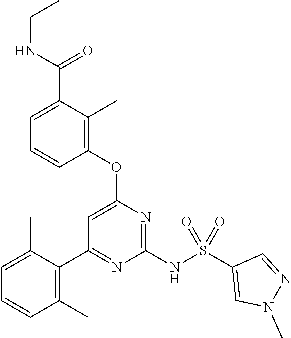 [SMILES: CCNC(=O)c1cccc(Oc2cc(-c3c(C)cccc3C)nc(NS(=O)(=O)c3cnn(C)c3)n2)c1C] | 1.5 | 520.189 | 521.23 | A |
| 1004 | 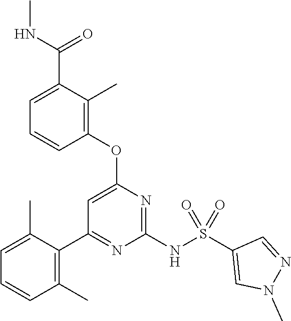 [SMILES: CNC(=O)c1cccc(Oc2cc(-c3c(C)cccc3C)nc(NS(=O)(=O)c3cnn(C)c3)n2)c1C] | 1.4 | 506.174 | 506.7 | A |
| 1005 | 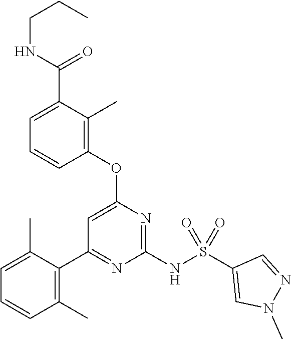 [SMILES: CCCNC(=O)c1cccc(Oc2cc(-c3c(C)cccc3C)nc(NS(=O)(=O)c3cnn(C)c3)n2)c1C] | 1.57 | 534.205 | 535.23 | A |
| 1006 | 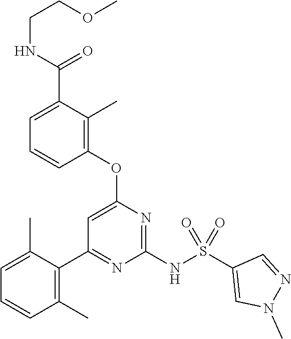 [SMILES: COCCNC(=O)c1cccc(Oc2cc(-c3c(C)cccc3C)nc(NS(=O)(=O)c3cnn(C)c3)n2)c1C] | 1.47 | 550.2 | 551.23 | A |
| 1007 | 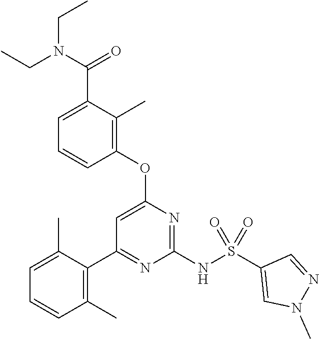 [SMILES: CCN(CC)C(=O)c1cccc(Oc2cc(-c3c(C)cccc3C)nc(NS(=O)(=O)c3cnn(C)c3)n2)c1C] | 1.62 | 548.221 | 549.24 | A |
| 1008 | 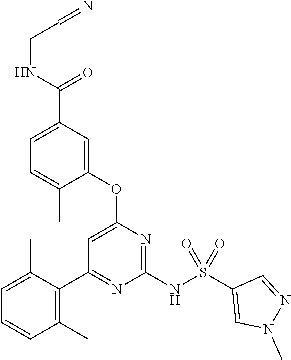 [SMILES: Cc1ccc(C(=O)NCC=N)cc1Oc1cc(-c2c(C)cccc2C)nc(NS(=O)(=O)c2cnn(C)c2)n1] | 1.53 | 531.169 | 532.26 | A |
| 1009 | 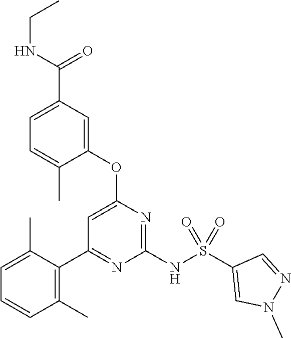 [SMILES: CCNC(=O)c1ccc(C)c(Oc2cc(-c3c(C)cccc3C)nc(NS(=O)(=O)c3cnn(C)c3)n2)c1] | 1.55 | 520.189 | 521.23 | A |
| 1010 | 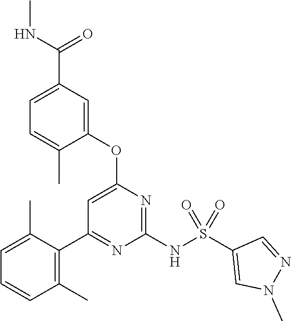 [SMILES: CNC(=O)c1ccc(C)c(Oc2cc(-c3c(C)cccc3C)nc(NS(=O)(=O)c3cnn(C)c3)n2)c1] | 1.47 | 506.174 | 506.67 | A |
| 1011 | 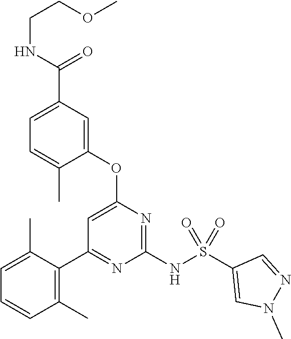 [SMILES: COCCNC(=O)c1ccc(C)c(Oc2cc(-c3c(C)cccc3C)nc(NS(=O)(=O)c3cnn(C)c3)n2)c1] | 1.5 | 550.2 | 551.2 | A |
| 1012 | 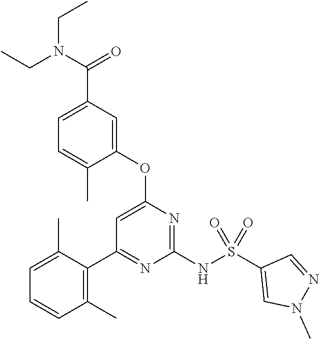 [SMILES: CCN(CC)C(=O)c1ccc(C)c(Oc2cc(-c3c(C)cccc3C)nc(NS(=O)(=O)c3cnn(C)c3)n2)c1] | 1.7 | 548.221 | 549.52 | A |
| 1013 | 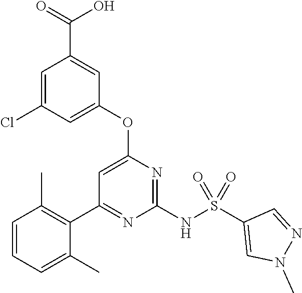 [SMILES: Cc1cccc(C)c1-c1cc(Oc2cc(Cl)cc(C(=O)O)c2)nc(NS(=O)(=O)c2cnn(C)c2)n1] | 1.62 | 513.087 | 515.37 | A |
| 1014 | 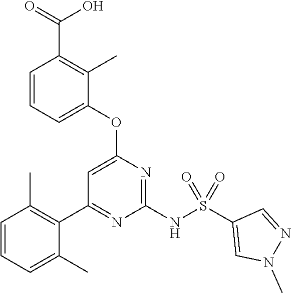 [SMILES: Cc1cccc(C)c1-c1cc(Oc2cccc(C(=O)O)c2C)nc(NS(=O)(=O)c2cnn(C)c2)n1] | 1.48 | 493.142 | 495.41 | A |
| 1015 | 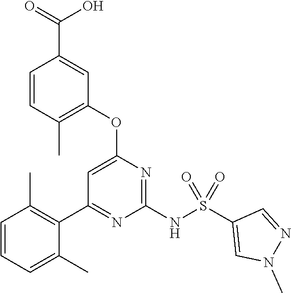 [SMILES: Cc1ccc(C(=O)O)cc1Oc1cc(-c2c(C)cccc2C)nc(NS(=O)(=O)c2cnn(C)c2)n1] | 1.51 | 493.142 | 495.41 | A |
| 1016 | 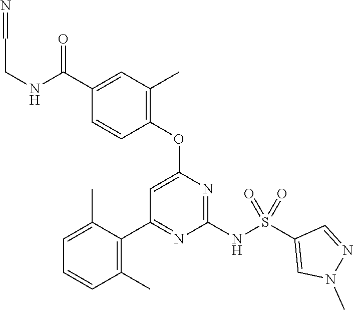 [SMILES: Cc1cc(C(=O)NCC=N)ccc1Oc1cc(-c2c(C)cccc2C)nc(NS(=O)(=O)c2cnn(C)c2)n1] | 1.47 | 531.169 | 533.47 | A |
| 1017 | 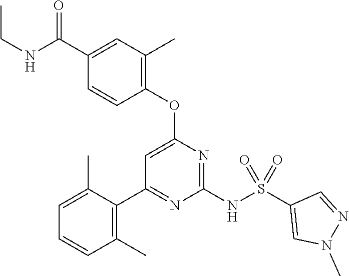 [SMILES: CCNC(=O)c1ccc(Oc2cc(-c3c(C)cccc3C)nc(NS(=O)(=O)c3cnn(C)c3)n2)c(C)c1] | 1.51 5 | 520.189 | 522.47 | A |
| 1018 | 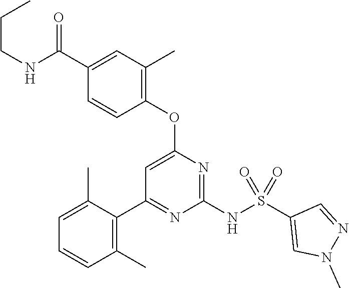 [SMILES: CCCNC(=O)c1ccc(Oc2cc(-c3c(C)cccc3C)nc(NS(=O)(=O)c3cnn(C)c3)n2)c(C)c1] | 1.61 | 534.205 | 536.52 | A |
| 1019 | 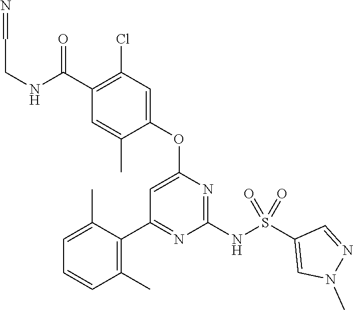 [SMILES: Cc1cc(C(=O)NCC=N)c(Cl)cc1Oc1cc(-c2c(C)cccc2C)nc(NS(=O)(=O)c2cnn(C)c2)n1] | 1.47 | 551.114 | 553.43 | A |
| 1020 | 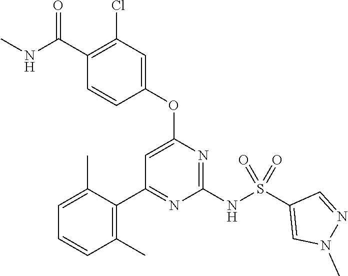 [SMILES: CNC(=O)c1ccc(Oc2cc(-c3c(C)cccc3C)nc(NS(=O)(=O)c3cnn(C)c3)n2)cc1Cl] | 1.43 | 526.119 | 528.43 | A |
| 1021 | 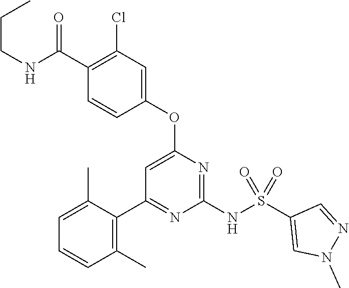 [SMILES: CCCNC(=O)c1ccc(Oc2cc(-c3c(C)cccc3C)nc(NS(=O)(=O)c3cnn(C)c3)n2)cc1Cl] | 1.63 | 554.15 | 556.47 | A |
| 1022 | 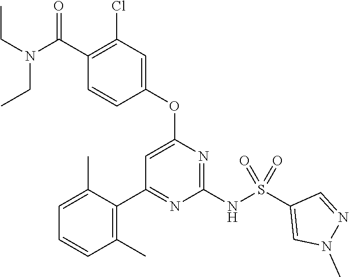 [SMILES: CCN(CC)C(=O)c1ccc(Oc2cc(-c3c(C)cccc3C)nc(NS(=O)(=O)c3cnn(C)c3)n2)cc1Cl] | 1.73 | 568.166 | 570.48 | A |
| 1023 | 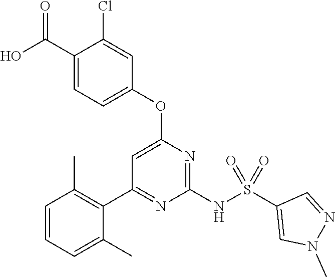 [SMILES: Cc1cccc(C)c1-c1cc(Oc2ccc(C(=O)O)c(Cl)c2)nc(NS(=O)(=O)c2cnn(C)c2)n1] | 1.53 | 513.087 | 515.4 | A |
| 1024 | 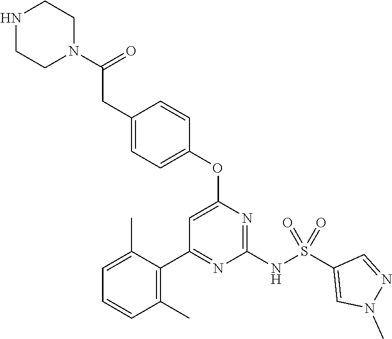 [SMILES: Cc1cccc(C)c1-c1cc(Oc2ccc(CC(=O)N3CCNCC3)cc2)nc(NS(=O)(=O)c2cnn(C)c2)n1] | 1.12 | 561.216 | 562.3 | A |
| 1025 | 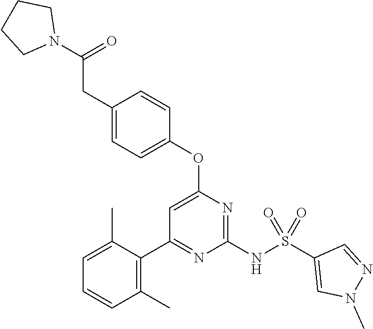 [SMILES: Cc1cccc(C)c1-c1cc(Oc2ccc(CC(=O)N3CCCC3)cc2)nc(NS(=O)(=O)c2cnn(C)c2)n1] | 1.5 | 546.205 | 547.3 | A |
| 1026 | 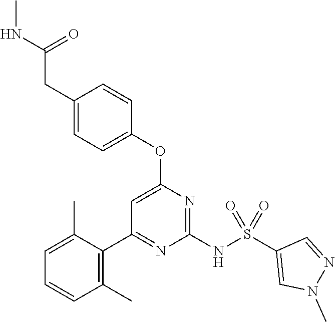 [SMILES: CNC(=O)Cc1ccc(Oc2cc(-c3c(C)cccc3C)nc(NS(=O)(=O)c3cnn(C)c3)n2)cc1] | 1.3 | 506.174 | 507.3 | A |
| 1027 | 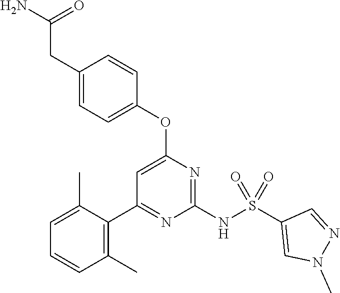 [SMILES: Cc1cccc(C)c1-c1cc(Oc2ccc(CC(N)=O)cc2)nc(NS(=O)(=O)c2cnn(C)c2)n1] | 1.25 | 492.158 | 493.3 | A |
| 1028 | 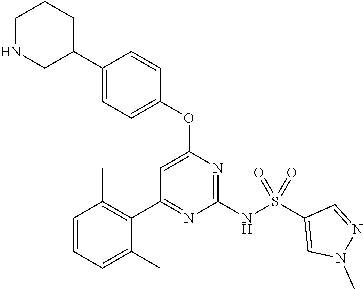 [SMILES: Cc1cccc(C)c1-c1cc(Oc2ccc(C3CCCNC3)cc2)nc(NS(=O)(=O)c2cnn(C)c2)n1] | 1.22 | 518.21 | 519.3 | A |
| 1029 | 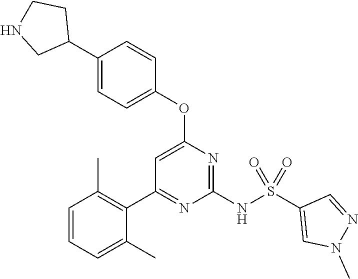 [SMILES: Cc1cccc(C)c1-c1cc(Oc2ccc(C3CCNC3)cc2)nc(NS(=O)(=O)c2cnn(C)c2)n1] | 1.12 | 504.194 | 505.2 | A |
| 1030 | 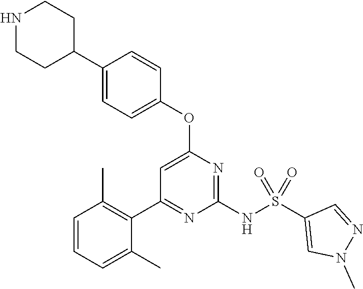 [SMILES: Cc1cccc(C)c1-c1cc(Oc2ccc(C3CCNCC3)cc2)nc(NS(=O)(=O)c2cnn(C)c2)n1] | 1.15 | 518.21 | 519.2 | A |
| 1031 | 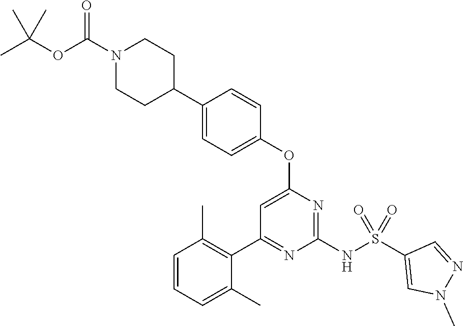 [SMILES: Cc1cccc(C)c1-c1cc(Oc2ccc(C3CCN(C(=O)OC(C)(C)C)CC3)cc2)nc(NS(=O)(=O)c2cnn(C)c2)n1] | 2.04 | 618.262 | 619.3 | A |
| 1032 | 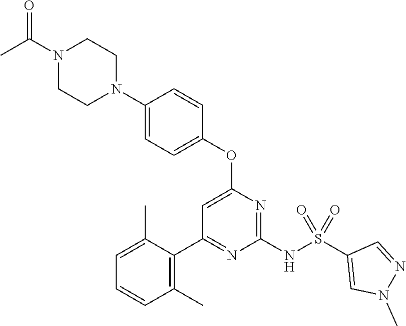 [SMILES: CC(=O)N1CCN(c2ccc(Oc3cc(-c4c(C)cccc4C)nc(NS(=O)(=O)c4cnn(C)c4)n3)cc2)CC1] | 1.42 | 561.216 | 562.2 | A |
| 1033 | 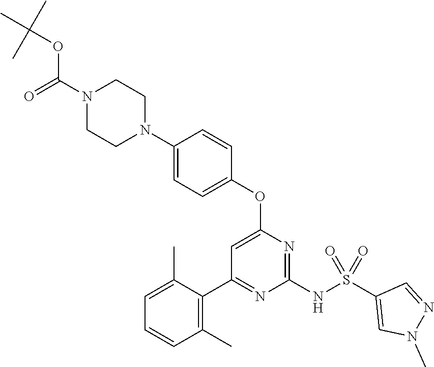 [SMILES: Cc1cccc(C)c1-c1cc(Oc2ccc(N3CCN(C(=O)OC(C)(C)C)CC3)cc2)nc(NS(=O)(=O)c2cnn(C)c2)n1] | 1.9 | 619.258 | 620.3 | A |
| 1034 | 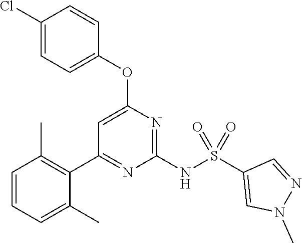 [SMILES: Cc1cccc(C)c1-c1cc(Oc2ccc(Cl)cc2)nc(NS(=O)(=O)c2cnn(C)c2)n1] | 1.78 | 469.098 | 470.1 | A |
| 1035 | 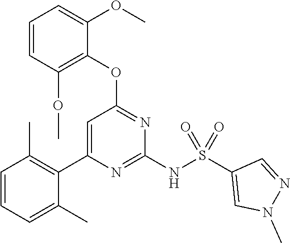 [SMILES: COc1cccc(OC)c1Oc1cc(-c2c(C)cccc2C)nc(NS(=O)(=O)c2cnn(C)c2)n1] | 1.6 | 495.158 | 496.2 | A |
| 1036 | 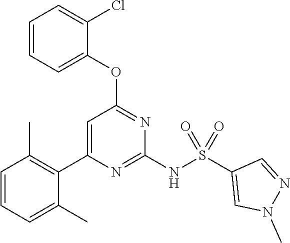 [SMILES: Cc1cccc(C)c1-c1cc(Oc2ccccc2Cl)nc(NS(=O)(=O)c2cnn(C)c2)n1] | 1.78 | 469.098 | 470.11 | A |
| 1037 | 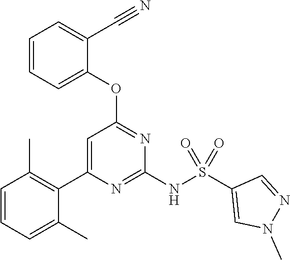 [SMILES: Cc1cccc(C)c1-c1cc(Oc2ccccc2C#N)nc(NS(=O)(=O)c2cnn(C)c2)n1] | 1.63 | 460.132 | 461.01 | A |
| 1038 | 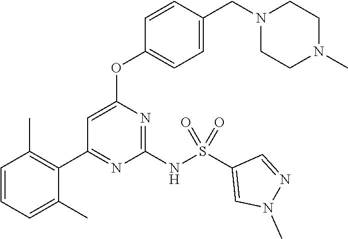 [SMILES: Cc1cccc(C)c1-c1cc(Oc2ccc(CN3CCN(C)CC3)cc2)nc(NS(=O)(=O)c2cnn(C)c2)n1] | 0.96 | 547.237 | 548.3 | A |
| 1039 | 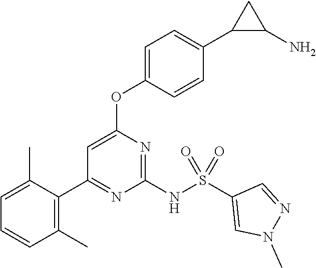 [SMILES: Cc1cccc(C)c1-c1cc(Oc2ccc(C3CC3N)cc2)nc(NS(=O)(=O)c2cnn(C)c2)n1] | 1.18 | 490.179 | 491.2 | A |
| 1040 | 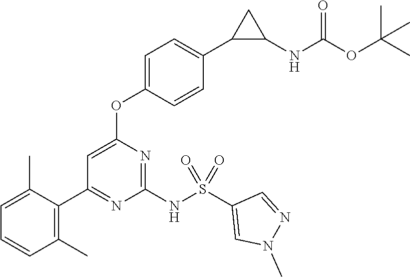 [SMILES: Cc1cccc(C)c1-c1cc(Oc2ccc(C3CC3NC(=O)OC(C)(C)C)cc2)nc(NS(=O)(=O)c2cnn(C)c2)n1] | 1.63 | 590.231 | 591.9 | A |
| 1041 | 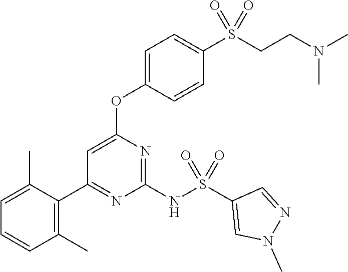 [SMILES: Cc1cccc(C)c1-c1cc(Oc2ccc(S(=O)(=O)CCN(C)C)cc2)nc(NS(=O)(=O)c2cnn(C)c2)n1] | 1.13 | 570.172 | 571.3 | A |
| 1042 | 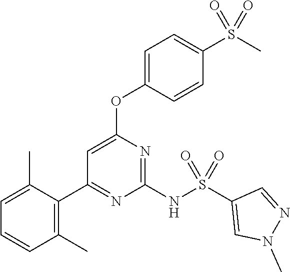 [SMILES: Cc1cccc(C)c1-c1cc(Oc2ccc(S(C)(=O)=O)cc2)nc(NS(=O)(=O)c2cnn(C)c2)n1] | 1.41 | 513.114 | 514.4 | A |
| 1043 | 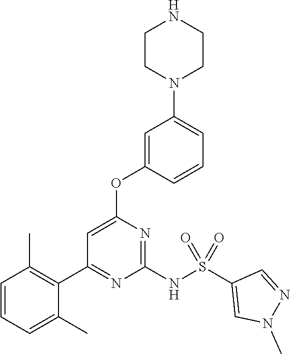 [SMILES: Cc1cccc(C)c1-c1cc(Oc2cccc(N3CCNCC3)c2)nc(NS(=O)(=O)c2cnn(C)c2)n1] | 1.16 | 519.205 | 520.3 | A |
| 1044 | 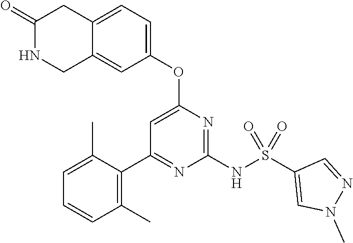 [SMILES: Cc1cccc(C)c1-c1cc(Oc2ccc3c(c2)CNC(=O)C3)nc(NS(=O)(=O)c2cnn(C)c2)n1] | 1.26 | 504.158 | 505.3 | A |
| 1045 | 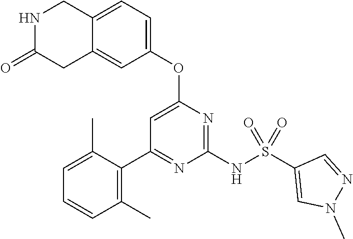 [SMILES: Cc1cccc(C)c1-c1cc(Oc2ccc3c(c2)CC(=O)NC3)nc(NS(=O)(=O)c2cnn(C)c2)n1] | 1.27 | 504.158 | 505.3 | A |
| 1046 | 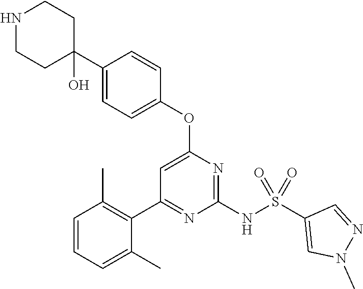 [SMILES: Cc1cccc(C)c1-c1cc(Oc2ccc(C3(O)CCNCC3)cc2)nc(NS(=O)(=O)c2cnn(C)c2)n1] | 1.05 | 534.205 | 535.5 | A |
| 1047 | 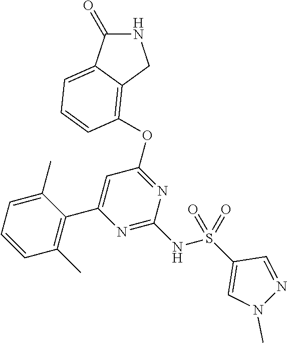 [SMILES: Cc1cccc(C)c1-c1cc(Oc2cccc3c2CNC3=O)nc(NS(=O)(=O)c2cnn(C)c2)n1] | 1.38 | 490.142 | 491.16 | A |
| 1048 | 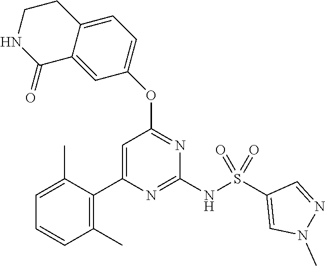 [SMILES: Cc1cccc(C)c1-c1cc(Oc2ccc3c(c2)C(=O)NCC3)nc(NS(=O)(=O)c2cnn(C)c2)n1] | 1.42 | 504.158 | 505.16 | A |
| 1049 | 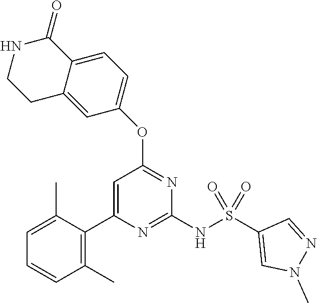 [SMILES: Cc1cccc(C)c1-c1cc(Oc2ccc3c(c2)CCNC3=O)nc(NS(=O)(=O)c2cnn(C)c2)n1] | 1.45 | 504.158 | 505.23 | A |
| 1050 | 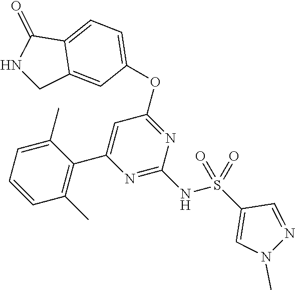 [SMILES: Cc1cccc(C)c1-c1cc(Oc2ccc3c(c2)CNC3=O)nc(NS(=O)(=O)c2cnn(C)c2)n1] | 1.37 | 490.142 | 491.19 | A |
| 1051 | 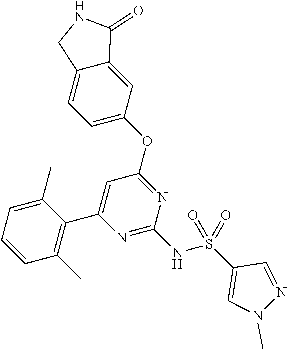 [SMILES: Cc1cccc(C)c1-c1cc(Oc2ccc3c(c2)C(=O)NC3)nc(NS(=O)(=O)c2cnn(C)c2)n1] | 1.35 | 490.142 | 490.67 | A |
| 1052 | 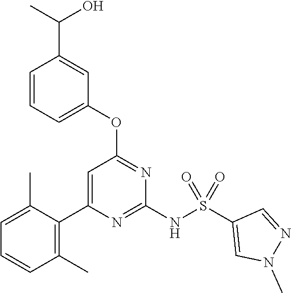 [SMILES: Cc1cccc(C)c1-c1cc(Oc2cccc(C(C)O)c2)nc(NS(=O)(=O)c2cnn(C)c2)n1] | 1.5 | 479.163 | 480.2 | A |
| 1053 | 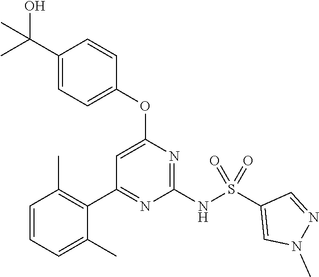 [SMILES: Cc1cccc(C)c1-c1cc(Oc2ccc(C(C)(C)O)cc2)nc(NS(=O)(=O)c2cnn(C)c2)n1] | 1.5 | 493.178 | 494.2 | A |
| 1054 | 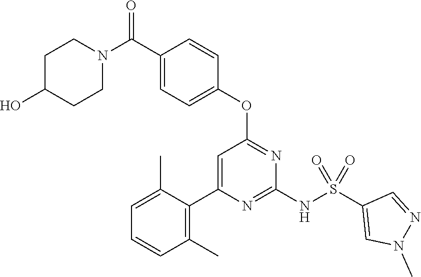 [SMILES: Cc1cccc(C)c1-c1cc(Oc2ccc(C(=O)N3CCC(O)CC3)cc2)nc(NS(=O)(=O)c2cnn(C)c2)n1] | 1.35 | 562.2 | 563.17 | A |
| 1055 | 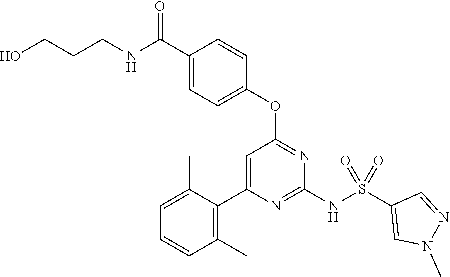 [SMILES: Cc1cccc(C)c1-c1cc(Oc2ccc(C(=O)NCCCO)cc2)nc(NS(=O)(=O)c2cnn(C)c2)n1] | 1.33 | 536.184 | 536.74 | A |
| 1056 | 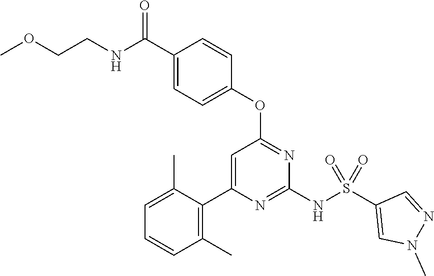 [SMILES: COCCNC(=O)c1ccc(Oc2cc(-c3c(C)cccc3C)nc(NS(=O)(=O)c3cnn(C)c3)n2)cc1] | 1.43 | 536.184 | 536.74 | A |
| 1057 | 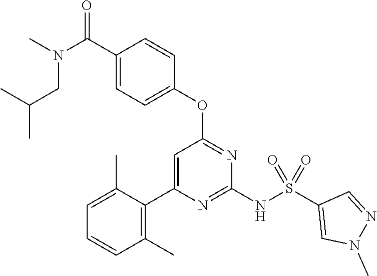 [SMILES: Cc1cccc(C)c1-c1cc(Oc2ccc(C(=O)N(C)CC(C)C)cc2)nc(NS(=O)(=O)c2cnn(C)c2)n1] | 1.68 | 548.221 | 548.78 | A |
| 1058 | 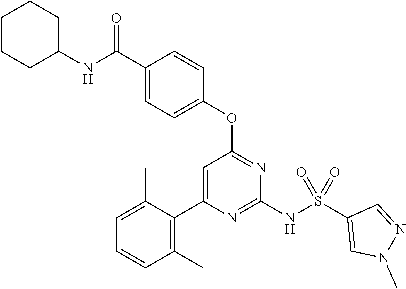 [SMILES: Cc1cccc(C)c1-c1cc(Oc2ccc(C(=O)NC3CCCCC3)cc2)nc(NS(=O)(=O)c2cnn(C)c2)n1] | 1.73 | 560.221 | 561.18 | A |
| 1059 | 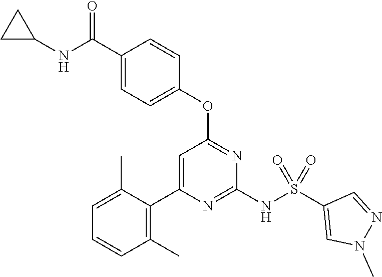 [SMILES: Cc1cccc(C)c1-c1cc(Oc2ccc(C(=O)NC3CC3)cc2)nc(NS(=O)(=O)c2cnn(C)c2)n1] | 1.48 | 518.174 | 518.85 | A |
| 1060 | 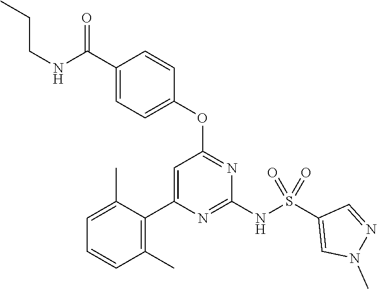 [SMILES: CCCNC(=O)c1ccc(Oc2cc(-c3c(C)cccc3C)nc(NS(=O)(=O)c3cnn(C)c3)n2)cc1] | 1.55 | 520.189 | 520.95 | A |
| 1061 | 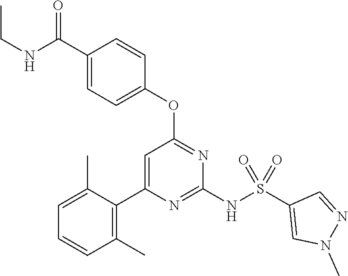 [SMILES: CCNC(=O)c1ccc(Oc2cc(-c3c(C)cccc3C)nc(NS(=O)(=O)c3cnn(C)c3)n2)cc1] | 1.47 | 506.174 | 506.98 | A |
| 1062 | 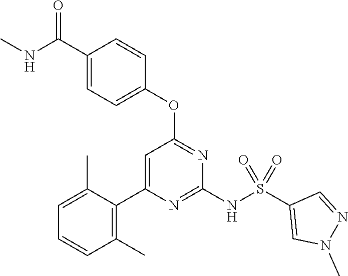 [SMILES: CNC(=O)c1ccc(Oc2cc(-c3c(C)cccc3C)nc(NS(=O)(=O)c3cnn(C)c3)n2)cc1] | 1.4 | 492.158 | 492.73 | A |
| 1063 | 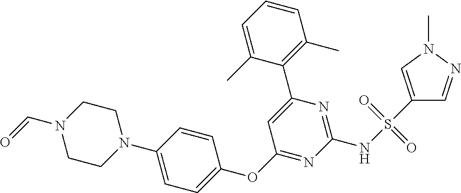 [SMILES: Cc1cccc(C)c1-c1cc(Oc2ccc(N3CCN(C=O)CC3)cc2)nc(NS(=O)(=O)c2cnn(C)c2)n1] | 1.42 | 547.2 | 548 | A |
| 1064 | 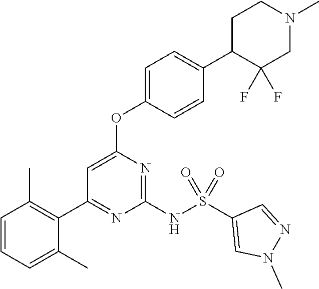 [SMILES: Cc1cccc(C)c1-c1cc(Oc2ccc(C3CCN(C)CC3(F)F)cc2)nc(NS(=O)(=O)c2cnn(C)c2)n1] | 1.18 | 568.207 | 569.4 | A |

| Compound | |
|---|---|
| Number | NMR |
| 934 | ¹H NMR (400 MHz, DMSO-d₆) δ 11.67 (s, 1H), 7.73 (d, J = 8.4 Hz, 2H), 7.60 (s, |
| | 1H), 7.37 (d, J = 8.4 Hz, 2H), 7.33 - 7.20 (m, 1H), 7.14 (d, J = 7.6 Hz, 2H), 6.72 |
| | (s, 1H), 6.66 (s, 1H), 3.73 (s, 3H), 2.03 (s, 6H), 1.74 (s, 3H). |
| 940 | ¹H NMR (400 MHz, DMSO-d₆) δ 10.70 (s, 1H), 7.73 (s, 1H), 7.33 (s, 1H), 7.27 - |
| | 7.21 (m, 1H), 7.20 - 7.09 (m, 4H), 6.88 (d, J = 9.2 Hz, 2H), 6.45 (s, 1H), 3.90 - |
| | 3.79 (m, 1H), 3.76 (s, 3H), 3.73 - 3.65 (m, 1H), 3.56 - 3.35 (m, 4H), 3.22 - 3.05 |
| | (m, 2H), 2.78 (dd, J = 5.0, 1.8 Hz, 3H), 2.36 - 2.24 (m, 2H), 2.23 - 2.11 (m,1H), |
| | 2.02 (s, 6H). |
| 943 | ¹H NMR (400 MHz, Chloroform-d) δ 7.39 - 7.37 (m, 2H), 7.25 - 7.20 (m, 3H), |
| | 7.14 - 7.08 (m, 4H), 6.44 (s, 1H), 3.77 (s, 3H), 3.65 (t, J = 6.0 Hz, 4H), 2.59 (t, J = |
| | 6.0 Hz, 5H), 2.11 (s, 6H). |
| 947 | ¹H NMR (400 MHz, DMSO-d₆) δ 10.33 (s, 1H), 7.95 - 7.37 (m, 1H), 7.27 - 7.20 |
| | (m, 4H), 7.18 - 7.10 (m, 4H), 6.56 (s, 1H), 3.95 - 3.78 (m, 2H), 3.75 (s, 3H), 3.66 - |
| | 3.53 (m, 2H), 3.18 - 3.06 (m, 2H), 2.97 - 2.84 (m, 1H), 2.04 (s, 7H), 1.18 - 1.02 |
| | (m, 3H), 0.93 - 0.73 (m, 3H). |
| 955 | ¹H NMR (400 MHz, MeOD-d₄) δ 9.80 (d, J = 0.9 Hz, 1H), 8.68 - 8.57 (m, 2H), |
| | 8.56 - 8.41 (m, 2H), 8.24 (dd, J = 9.0, 2.4 Hz, 1H), 7.69 (s, 1H), 7.27 (dd, J = 8.3, |
| | 6.9 Hz, 1H), 7.17 (d, J = 7.3 Hz, 2H), 6.93 (d, J = 0.7 Hz, 1H), 6.76 (s, 1H), 3.71 |
| | (s, 3H), 2.15 (s, 6H). |
| 956 | ¹H NMR (400 MHz, DMSO-d₆) δ 11.67 (s, 1H), 7.82 (d, J = 8.4 Hz, 2H), 7.76 - |
| | 7.69 (m, 2H), 7.49 (dd, J = 8.4, 6.9 Hz, 2H), 7.45 - 7.35 (m, 3H), 7.31 - 7.21 (m, |
| | 2H), 7.14 (d, J = 7.6 Hz, 2H), 6.68 (s, 1H), 3.72 (s, 3H), 2.04 (s, 6H). |
| 957 | ¹H NMR (400 MHz, DMSO-d₆) δ 9.30 (dd, J = 5.1, 1.5 Hz, 1H), 9.06 (dd, J = 8.6, |
| | 1.4 Hz, 1H), 8.54 (d, J = 9.2 Hz, 1H), 8.35 (d, J = 2.6 Hz, 1H), 8.17 - 8.03 (m, |
| | 2H), 7.75 (s, 1H), 7.34 - 7.21 (m, 1H), 7.19 - 7.08 (m, 3H), 6.86 (s, 1H), 3.67 (s, |
| | 3H), 2.07 (s, 6H). |
| 966 | ¹H NMR (400 MHz, DMSO-d₆) δ 7.64 (s, 1H), 7.35 (s, 4H), 7.32 - 7.21 (m, 2H), |
| | 7.14 (d, J = 7.6 Hz, 2H), 6.62 (s, 1H), 3.76 (s, 3H), 3.51 (t, J = 6.1 Hz, 2H), 3.28 |
| | (s, 2H), 2.04 (s, 6H), 1.80 (p, J = 6.3 Hz, 2H). |
| 967 | ¹H NMR (400 MHz, DMSO-d₆) δ 7.63 (s, 1H), 7.30 - 7.19 (m, 2H), 7.11 (dd, J = |
| | 15.4, 8.3 Hz, 4H), 6.82 - 6.70 (m, 2H), 6.51 (s, 1H), 4.02 - 3.95 (m, 3H), 3.76 (s, |
| | 3H), 3.40 (dd, J = 14.2, 5.3 Hz, 1H), 3.17 (dd, J = 14.2, 6.8 Hz, 1H), 3.02 (s, 3H), |
| | 2.04 (s, 6H), 1.32 (d, J = 6.4 Hz, 3H). [2] |
| 970 | ¹H NMR (400 MHz, DMSO-d₆) δ 7.73 - 7.49 (m, 0H), 7.32 (s, 0H), 7.24 (dd, J = |
| | 8.2, 6.9 Hz, 0H), 7.13 (d, J = 7.6 Hz, 1H), 3.75 (s, 1H), 3.12 (s, 1H), 2.03 (s, 3H), |
| | 1.68 (dt, J = 13.4, 6.6 Hz, 0H), 1.48 (q, J = 7.2 Hz, 1H), 0.89 (d, J = 6.6 Hz, 3H). |
| 981 | ¹H NMR (400 MHz, DMSO-d₆) δ 9.40 - 9.22 (m, 1H), 9.05 - 8.91 (m, 1H), 7.62 |
| | (s, 1H), 7.29 - 7.20 (m, 4H),7.18 - 7.08 (m, 4H)), 6.55 (s, 1H), 3.97 - 3.69 (m, 5H), |
| | 3.46 - 3.28 (m, 2H), 3.21 - 3.08 (m, 1H), 3.03 - 2.95 (m, 1H), 2.87 - 2.70 (m, 1H), |
| | 2.04 (s, 6H), 1.30 (d, J = 6.5 Hz, 3H). |
| 982 | ¹H NMR (400 MHz, DMSO-d₆) δ 9.43 (s, 1H), 9.02 (s, 1H), 7.65 (s, 1H), 7.31 - |
| | 7.19 (m, 4H), 7.19 - 7.11 (m, 4H), 6.57 (s, 1H), 4.08 (s, 1H), 3.76 (s, 3H), 3.54 - |
| | 3.37 (m, 1H), 3.37 - 3.24 (m, 2H), 3.23 - 3.07 (m, 3H), 2.04 (s, 6H), 1.06 (d, J = |
| | 6.7 Hz, 3H). |
| 983 | ¹H NMR (400 MHz, DMSO-d₆) δ 9.36 (s, 1H), 9.18 (s, 1H), 7.72 (s, 1H), 7.41 - |
| | 7.19 (m, 5H), 7.18 - 7.07 (m, 3H), 6.61 (s, 1H), 3.76 (s, 3H), 3.52 - 3.48 (m, 2H), |
| | 3.46 - 3.35 (m, 1H), 3.35 - 3.25 (m, 1H), 3.10 - 2.94 (m, 1H), 2.92 - 2.83 (m, 1H), |
| | 2.05 (s, 6H), 1.26 (d, J = 6.5 Hz, 3H), 0.97 (d, J = 6.1 Hz, 3H). |
| 984 | ¹H NMR (400 MHz, DMSO-d₆) δ 8.97 - 8.77 (m, 2H), 8.26 (d, J = 5.8 Hz, 2H), |
| | 8.16 (d, J = 8.8 Hz, 2H), 7.78 (s, 1H), 7.62 - 7.52 (m,2H), 7.31 - 7.20 (m, 2H), |
| | 7.15 (d, J = 7.6 Hz, 2H), 6.74 (s, 1H), 3.72 (s, 3H), 2.06 (s,6H). |
| 989 | ¹H NMR (400 MHz, DMSO-d₆) δ 9.82 (s, 1H), 8.91 (s, 1H), 7.75 - 7.60 (m, 2H), |
| | 7.51 - 7.38 (m, 2H), 7.29 - 7.21 (m, 2H), 7.14 (d, J = 7.6 Hz, 2H), 6.63 (s, 1H), |
| | 4.72 - 4.54 (m, 1H), 3.75 (s, 3H), 3.46 - 3.22 (m, 2H), 2.45 - 2.36 (m, 1H), 2.21 - |
| | 1.93 (m, 7H). |
| 991 | ¹H NMR (400 MHz, DMSO-d₆) δ 7.64 (s, 1H), 7.58 (d, J = 8.2 Hz, 2H), 7.38 (d, |
| | J = 8.0 Hz, 2H), 7.31 - 7.19 (m, 1H), 7.14 (d, J = 7.6 Hz, 2H), 6.62 (s, 1H), 6.40 |
| | (d, J = 4.4 Hz, 1H), 5.20 - 5.04 (m, 1H), 3.75 (s, 3H), 3.30 - 3.24 (m, 2H), 2.87 (s, |
| | 6H), 2.04 (s, 6H). |
| 992 | ¹H NMR (400 MHz, DMSO-d₆) δ 9.09 (s, 1H), 8.73 (s, 1H), 7.63 - 7.54 (m, 2H), |
| | 7.37 (d, J = 8.5 Hz, 2H), 7.25 (t, J = 7.6 Hz, 1H), 7.14 (d, J = 7.6 Hz, 2H), 6.63 |
| | (s, 1H), 4.46 - 3.99 (m, 5H), 3.74 (s, 3H), 2.04 (s, 6H). |
| 994 | ¹H NMR (400 MHz, DMSO-d₆) δ 10.16 (s, 1H), 8.11 - 8.02 (m, 2H), 7.78 (s, 1H), |
| | 7.70 - 7.59 (m, 2H), 7.32 (s, 1H), 7.26 (dd, J = 8.2, 6.9 Hz, 1H), 7.15 (d, J = 7.6 |
| | Hz, 2H), 6.74 (s, 1H), 3.74 (s, 3H), 2.06 (s, 6H). |
| 995 | ¹H NMR (400 MHz, DMSO-d₆) δ 11.73 (s, 1H), 8.07 - 7.70 (m, 3H), 7.63 (d, J = |
| | 8.4 Hz, 2H), 7.33 - 7.21 (m, 2H), 7.15 (d, J = 7.6 Hz, 2H), 6.78 (s, 1H), 3.76 (s, |
| | 3H), 2.59 (s, 3H), 2.06 (s, 6H). |
| 1065 | ¹H NMR (400 MHz, Methanol-d₄) δ 7.71 (s, 2H), 7.51 - 7.43 (m, 3H), 7.41 (s, 1H), |
| | 7.27 (dd, J = 8.3, 6.9 Hz, 1H), 7.20 - 7.12 (m, 2H), 6.59 (s, 1H), 3.80 (s, 3H), 2.85 |
| | (s, 1H), 2.15 (s, 6H), 1.77 (s, 2H), 1.13 (s, 2H). |
| 1066 | ¹H NMR (400 MHz, Methanol-d₄) δ 7.82 (dd, J = 8.3, 1.4 Hz, 2H), 7.73 (dt, J = |
| | 7.6, 1.4 Hz, 1H), 7.67 (t, J = 7.8 Hz, 1H), 7.64 - 7.58 (m, 2H), 7.55 (ddd, J = 8.0, |
| | 2.4, 1.2 Hz, 1H), 7.52 - 7.46 (m, 3H), 7.25 (d, J = 0.7 Hz, 1H), 7.20 (dd, J = 8.3, |
| | 6.9 Hz, 1H), 7.09 (dt, J = 7.5, 0.7 Hz, 2H), 6.53 (s, 1H), 3.72 (s, 3H), 2.07 (s, 6H). |
| 1067 | ¹H NMR (400 MHz, Methanol-d₄) δ 7.91 - 7.85 (m, 2H), 7.75 (dd, J = 8.3, 1.4 Hz, |
| | 2H), 7.61 - 7.54 (m, 1H), 7.49 - 7.45 (m, 2H), 7.44 (d, J = 4.6 Hz, 1H), 7.42 - 7.36 |
| | (m, 2H), 7.22 (d, J = 0.7 Hz, 1H), 7.16 (dd, J = 8.3, 6.9 Hz, 1H), 7.06 (dd, J = 7.4, |
| | 0.9 Hz, 2H), 6.52 (s, 1H), 3.68 (s, 3H), 2.04 (s, 6H). |
| 1068 | ¹H NMR (400 MHz, Methanol-d₄) δ 7.95 - 7.85 (m, 2H), 7.39 (d, J = 16.8 Hz, |
| | 2H), 7.27 (dd, J = 8.3, 6.9 Hz, 1H), 7.22 - 7.09 (m, 4H), 6.56 (d, J = 2.0 Hz, 1H), |
| | 3.80 (s, 3H), 2.14 (s, 6H). |
| 1063 | ¹H NMR (400 MHz, DMSO-d₆) δ 11.63 (s, 1H), 8.09 (s, 1H), 7.68 (s, 1H), 7.48 - |
| | 6.98 (m, 8H), 6.53 (s, 1H), 3.75 (s, 3H), 3.54 (dt, J = 11.1, 6.1 Hz, 4H), 3.16 (dt, |
| | J = 24.7, 5.2 Hz, 4H), 2.03 (s, 6H). |

### Example 292: Preparation of Compound 1069

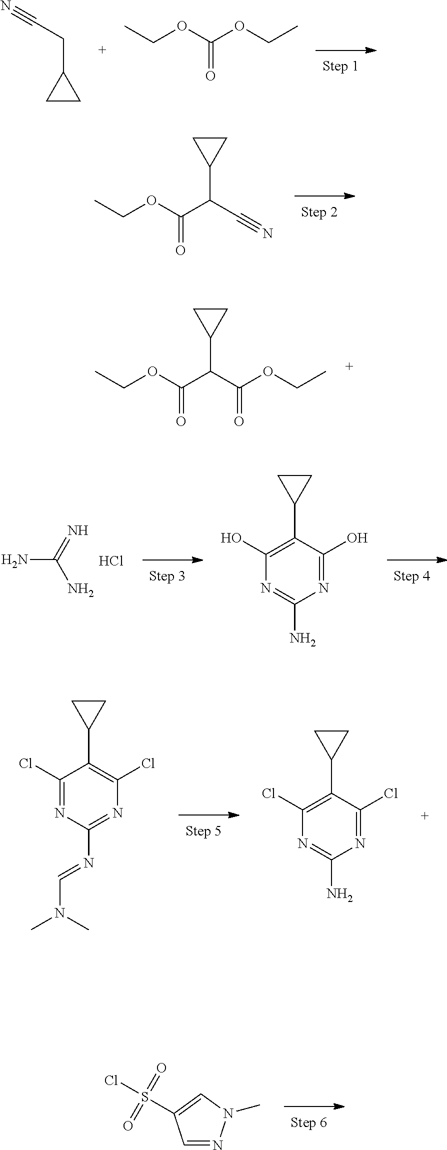

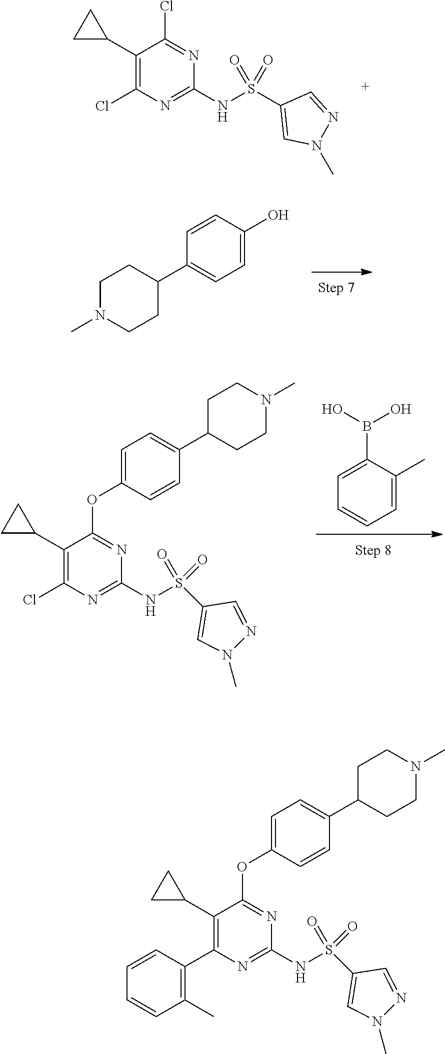

### Step 1: Ethyl 2-cyano-2-cyclopropyl-acetate

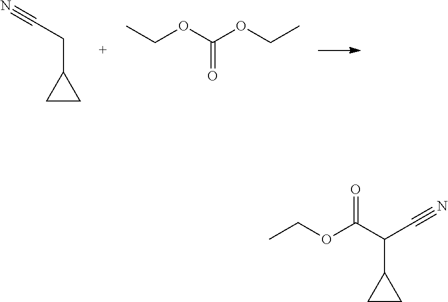

2-Cyclopropylacetonitrile (10 g, 123.28 mmol) dissolved in toluene (35 mL) was slowly added to diethyl carbonate (31.200 g, 32 mL, 264.11 mmol) and sodium hydride (10.9 g, 272.53 mmol) in refluxing toluene (75 mL). The reaction was maintained at reflux for another 2 hours. The reaction mixture was cooled to room temperature then transferred into acetic acid (50 mL) in an Erlenmeyer cooled with ice water. The resulting suspension was partitioned between water (200 mL) and ethyl acetate (500 mL). The organic phase was separated, washed with distilled water (2×100 mL). The organic layer was dried over anhydrous sodium sulfate, filtered and concentrated under reduced pressure. The resulting residue was partitioned between heptane (50 mL) and acetonitrile (100 mL). The lower phase (acetonitrile) was separated and washed with pentane (100 mL), separated then concentrated under reduced pressure to provide the desired product ethyl 2-cyano-2-cyclopropyl-acetate (2.2 g, 93%) brown oil, ESI-MS m/z calc. 153.079, found 154.2 (M+1)⁺; Retention time: 1.61 minutes, (LC method C), ¹H NMR (300 MHz, CDCl₃) ppm 0.46-0.64 (m, 2H), 0.64-0.83 (m, 2H), 1.24-1.44 (m, 4H), 3.22 (d, J=7.3 Hz, 1H), 4.20-4.35 (m, 2H).

### Step 2: Diethyl 2-cyclopropylpropanedioate

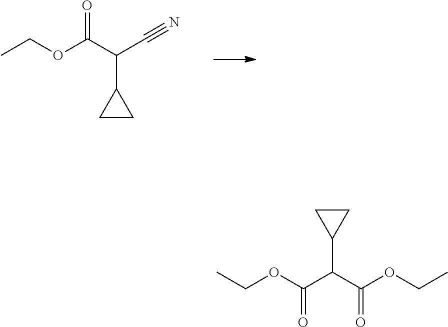

In a sealed tube under nitrogen atmosphere, dry ethanol (20.120 g, 25.5 mL, 436.74 mmol), chlorotrimethylsilane (22.7 g, 208.94 mmol) and ethyl 2-cyano-2-cyclopropyl-acetate (10.000 g, 65.284 mmol) was heated at 90° C. for 18 hours. The reaction mixture was cooled to room temperature and water (3.9000 g, 3.9 mL, 216.48 mmol) was added. The mixture was stirred for 1 minute and it was partitioned between water (100 mL) and ethyl acetate (300 mL). The organics were separated, dried over magnesium sulfate then concentrated under reduced pressure to provide the desired product diethyl 2-cyclopropylpropanedioate (12.01 g, 92%) as a brown oil, ESI-MS m/z calc. 200.1049, found 201.2 (M+1)⁺; Retention time: 1.84 minutes, (LC method C). ¹H NMR (300 MHz, CDCl₃) ppm −0.04-0.12 (m, 2H), 0.30-0.44 (m, 2H), 0.98 (t, J=7.0 Hz, 6H), 1.05-1.19 (m, 1H), 2.30 (d, J=10.0 Hz, 1H), 3.92 (q, J=7.1 Hz, 4H).

### Step 3: 2-Amino-5-cyclopropyl-pyrimidine-4,6-diol

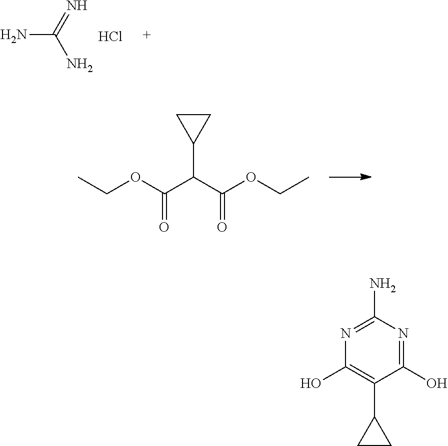

Under nitrogen atmosphere, heptane washed sodium hydride (6.8 g, 295.78 mmol) was dissolved in absolute ethanol (300 mL) and guanidine hydrochloride (11.67 g, 122.16 mmol) was added and the resulting suspension was stirred 5 minutes, then diethyl 2-cyclopropylpropanedioate (19.9 g, 99.385 mmol) was added and the mixture became very thick and hard to mix. The reaction was then left stirring at 80° C. overnight. The reaction mixture was concentrated under reduced pressure and the resulting residue was dissolved in deionized water (150 mL) and the resulting solution was acidified with HCl 13 N (˜15 mL) and the resulting precipitate, filtered, washed with water (2×15 mL). The resulting powder was dried under reduced pressure to provide desired 2-amino-5-cyclopropyl-pyrimidine-4,6-diol hydrate (17.5 g, 95%). ESI-MS m/z calc. 167.0695, found 168.2 (M+1)⁺+; Retention time: 0.382 minutes (LC method C). ¹H NMR (300 MHz, DMSO-d₆) ppm 0.35-0.51 (m, 2H), 0.75 (q, J=4.3 Hz, 2H), 1.31-1.51 (m, 1H), 6.29 (br. s., 2H), 10.18 (br. s., 2H).

### Step 4: N′-(4,6-Dichloro-5-cyclopropyl-pyrimidin-2-yl)-N,N-dimethyl-formamidine

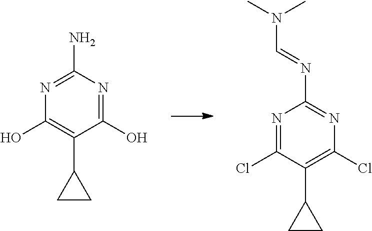 [SMILES: Nc1nc(O)c(C2CC2)c(O)n1 ; CN(C)/C=N/c1nc(Cl)c(C2CC2)c(Cl)n1]

Oxalyl chloride (127.31 g, 87.5 mL, 1.0030 mol) was slowly added to DMF (73.632 g, 78 mL, 1.0074 mol) in chloroform (700 mL) at room temperature. This mixture was stirred at room temperature for 30 minutes. 2-Amino-5-cyclopropyl-pyrimidine-4,6-diol (16.7 g, 99.901 mmol) was added then the reaction was stirred at 70° C. overnight. The reaction mixture was cooled and then added to saturated aqueous sodium bicarbonate (2 L) with strong stirring; solid sodium bicarbonate was added until neutral pH. The aqueous phase was separated and extracted with ethyl acetate (3×300 mL), the organic fractions were combined, dried over anhydrous sodium sulfate, filtered and concentrated under reduced pressure (using heptane azeotrope to drive out remaining traces of DMF) then left to dry under high vacuum to provide the desired product N′-(4,6-dichloro-5-cyclopropyl-pyrimidin-2-yl)-N,N-dimethyl-formamidine (23.04 g, 86%) as a dark solid ESI-MS m/z calc. 258.0439, found 259.1 (M+1)⁺; Retention time: 1.28 minutes (LC method C). ¹H NMR (300 MHz, CDCl₃) ppm 0.64-0.79 (m, 2H), 1.03-1.20 (m, 2H), 1.58-1.77 (m, 1H), 3.14 (d, J=2.6 Hz, 6H), 8.60 (s, 1H). This material was used in the next step without further purification.

### Step 5: 4,6-Dichloro-5-cyclopropyl-pyrimidin-2-amine

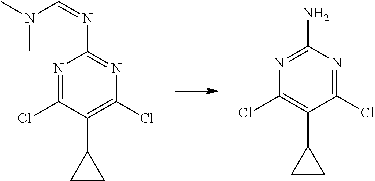 [SMILES: CN(C)/C=N\c1nc(Cl)c(C2CC2)c(Cl)n1 ; Nc1nc(Cl)c(C2CC2)c(Cl)n1]

To N′-(4,6-dichloro-5-cyclopropyl-pyrimidin-2-yl)-N,N-dimethyl-formamidine (12 g, 46.308 mmol) dissolved in isopropanol (240 mL) was added HCl (23 mL of 12 M, 276.00 mmol), the solution was left stirring at 50° C. for 2 hours. It was heated for an additional 30 minutes and the reaction was left to cool to room temperature, a precipitate formed. The mixture was cooled in an ice bath and the precipitate was filtered and washed with cold isopropanol then dried under vacuum to provide the desired 4,6-dichloro-5-cyclopropyl-pyrimidin-2-amine (6.4 g, 66%) as a beige powder ESI-MS m/z calc. 203.0017, found 204.1 (M+1)⁺; Retention time: 2.39 minutes (LC method H). ¹H NMR (300 MHz, DMSO-d₆) ppm 0.51-0.68 (m, 2H), 0.91-1.07 (m, 2H), 1.56 (tt, J=8.3, 5.4 Hz, 1H), 7.33 (s, 2H). The filtrate was concentrated under reduced pressure then partitioned between ethyl acetate (200 mL) and dilute sodium bicarbonate (100 mL), the organic phase was separated, and the aqueous phase was extracted with ethyl acetate (2×200 mL). The organic phases were combined, dried over anhydrous sodium sulfate, filtered and concentrated under reduced pressure and the resulting residue was purified on silica gel using 10% ethyl acetate in heptane to give additional desired product 4,6-dichloro-5-cyclopropyl-pyrimidin-2-amine (1.28 g, 14%) as a white powder, ESI-MS m/z calc. 203.0017, found 204.1 (M+1)⁺+; Retention time: 2.39 minutes (LC method H).

### Step 6: N-(4,6-Dichloro-5-cyclopropyl-pyrimidin-2-yl)-1-methyl-pyrazole-4-sulfonamide

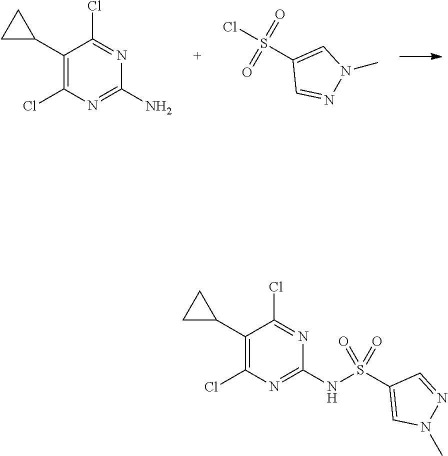

To a solution of 4,6-dichloro-5-cyclopropyl-pyrimidin-2-amine (3.00 g, 13.67 mmol) in DMF (54.68 mL) at 0° C. was added sodium hydride (approximately 2.187 g of 60% w/w, 54.68 mmol). After 15 minutes, 1-methylpyrazole-4-sulfonyl chloride (approximately 4.938 g, 27.34 mmol) was added and the reaction mixture was further stirred for 15 minutes at 0° C. before quenching with acetic acid (approximately 9.849 g, 9.327 mL, 164.0 mmol). The crude solution was partitioned between brine and ethyl acetate. The water layer was removed, and the organic layer was further washed with brine (2×). The combined organics were dried over magnesium sulfate, filtered, and concentrated in vacuo. The residual acetic acid was removed through azeotrope distillation with benzene (in vacuo). The crude residue was triturated with acetone to afford N-(4,6-dichloro-5-cyclopropyl-pyrimidin-2-yl)-1-methyl-pyrazole-4-sulfonamide (4.07 g, 86%) as a white solid. ESI-MS m/z calc. 347.00104, found 348.01 (M+1)⁺; Retention time: 0.58 minutes (LC method D).

### Step 7: N-[4-Chloro-5-cyclopropyl-6-[4-(1-methyl-4-piperidyl)phenoxy]pyrimidin-2-yl]-1-methyl-pyrazole-4-sulfonamide

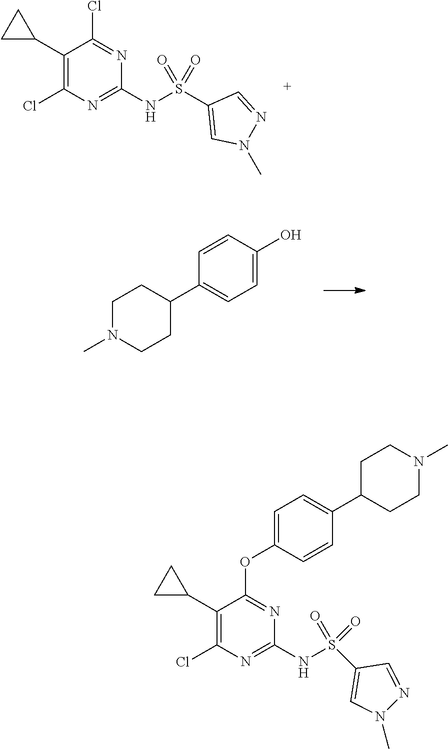 [SMILES: Cn1cc(S(=O)(=O)Nc2nc(Cl)c(C3CC3)c(Cl)n2)cn1 ; CN1CCC(c2ccc(O)cc2)CC1 ; CN1CCC(c2ccc(Oc3nc(NS(=O)(=O)c4cnn(C)c4)nc(Cl)c3C3CC3)cc2)CC1]

A heterogeneous mixture of N-(4,6-dichloro-5-cyclopropyl-pyrimidin-2-yl)-1-methyl-pyrazole-4-sulfonamide (approximately 306.1 mg, 0.8790 mmol), 4-(1-methyl-4-piperidyl)phenol (acetate salt) (approximately 220.9 mg, 0.8790 mmol), and potassium carbonate (approximately 485.9 mg, 3.516 mmol) in NMP (1.758 mL) was heated in a sealed vial to 110° C. for 16 hours. The reaction mixture was acidified with acetic acid (1 mL, 17.58 mmol), diluted with water (0.20 mL), and filtered through a 0.20 m filter syringe. The sample was purified by reverse phase HPLC (Waters Sunfire C₁₈ column (100×50 mm, 10 m particle size), gradient: 1-99% acetonitrile in water (5 mM HCl) over 15.0 minutes) to afford N-[4-chloro-5-cyclopropyl-6-[4-(1-methyl-4-piperidyl)phenoxy]pyrimidin-2-yl]-1-methyl-pyrazole-4-sulfonamide (Trifluoroacetate salt) (110 mg, 20%) as a gel. ESI-MS m/z calc. 502.1554, found 503.27 (M+1)⁺; Retention time: 0.53 minutes (LC method D).

### Step 8: N-[5-cyclopropyl-4-[4-(1-methyl-4-piperidyl)phenoxy]-6-(o-tolyl)pyrimidin-2-yl]-1-methyl-pyrazole-4-sulfonamide

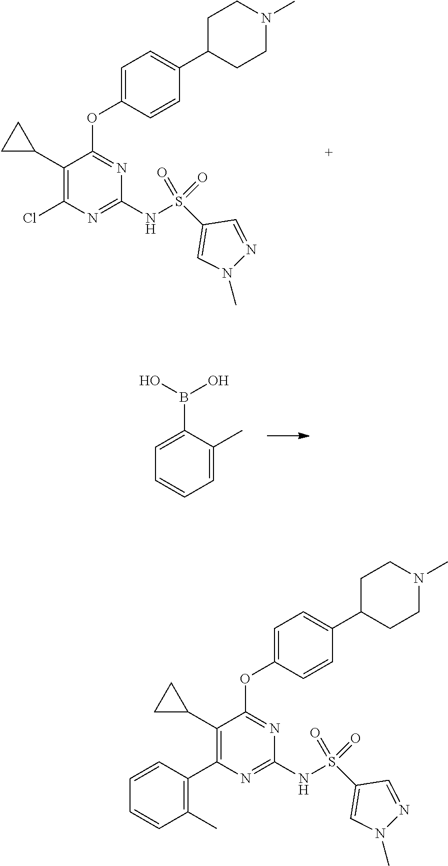

A heterogeneous mixture of N-[4-chloro-5-cyclopropyl-6-[4-(1-methyl-4-piperidyl)phenoxy]pyrimidin-2-yl]-1-methyl-pyrazole-4-sulfonamide (trifluoroacetate salt) (20.0 mg, 0.03241 mmol), o-tolylboronic acid (8.8 mg, 0.06473 mmol), tetrakis(triphenylphosphine)palladium(0) (7.5 mg, 0.006490 mmol) and potassium carbonate (17.9 mg, 0.1295 mmol) in dioxane (135 μL) and water (30 μL) was microwaved in a sealed vial to 125° C. for 30 minutes. The reaction mixture was acidified with acetic acid (60.0 μL, 1.055 mmol), diluted with DMSO (0.5 mL), and filtered through a 0.45 m filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[5-cyclopropyl-4-[4-(1-methyl-4-piperidyl)phenoxy]-6-(o-tolyl)pyrimidin-2-yl]-1-methyl-pyrazole-4-sulfonamide (hydrochloride salt) (8.8 mg, 42%) as a white solid. ESI-MS m/z calc. 558.24133, found 559.46 (M+1)⁺; Retention time: 1.28 minutes (LC method A).

### Example 293: Preparation of Compound 1070

### Step 1: N-[4-chloro-5-cyclopropyl-6-(2-methylphenoxy)pyrimidin-2-yl]-1-methyl-pyrazole-4-sulfonamide

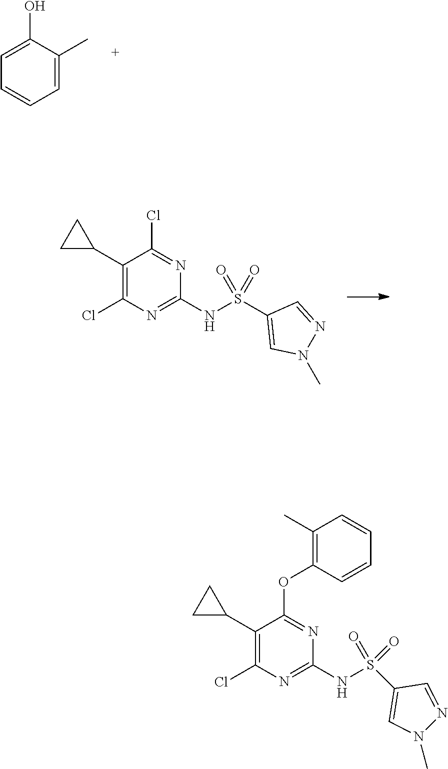

A heterogeneous mixture of o-cresol (approximately 98.84 mg, 178.7 μL, 0.9140 mmol), potassium carbonate (approximately 505.3 mg, 3.656 mmol), and N-(4,6-dichloro-5-cyclopropyl-pyrimidin-2-yl)-1-methyl-pyrazole-4-sulfonamide (approximately 318.3 mg, 0.9140 mmol) in NMP (1.828 mL) was heated in a sealed vial to 110° C. The reaction mixture was acidified with acetic acid (approximately 823.3 mg, 779.6 μL, 13.71 mmol), diluted with water (0.2 mL), and filtered through a 0.2 m syringe filter. The sample was purified by reverse phase HPLC (Waters Sunfire C₁₈ column (100×50 mm, 10 m particle size), gradient: 1-99% acetonitrile in water (5 mM HCl) over 15.0 minutes) to afford N-[4-chloro-5-cyclopropyl-6-(2-methylphenoxy)pyrimidin-2-yl]-1-methyl-pyrazole-4-sulfonamide (76.3 mg, 20%) as a white solid. ESI-MS m/z calc. 419.08188, found 420.18 (M+1)⁺; Retention time: 0.72 minutes (LC method D).

### Step 2: N-[5-cyclopropyl-4-(2-methylphenoxy)-6-(o-tolyl)pyrimidin-2-yl]-1-methyl-pyrazole-4-sulfonamide

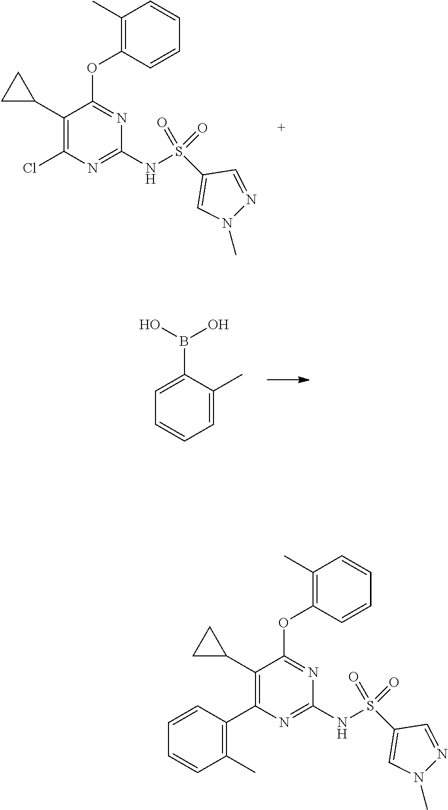

A heterogeneous solution of N-[4-chloro-5-cyclopropyl-6-(2-methylphenoxy)pyrimidin-2-yl]-1-methyl-pyrazole-4-sulfonamide (20.00 mg, 0.04763 mmol), o-tolylboronic acid (13.0 mg, 0.09562 mmol), potassium carbonate (19.8 mg, 0.1433 mmol), and tetrakis(triphenylphosphine)palladium(0) (11.0 mg, 0.009519 mmol) in dioxane (160 μL) and water (30 μL) was microwaved in a sealed vial to 125° C. for 30 minutes. The reaction mixture was acidified with acetic acid (60 μL, 1.055 mmol), diluted with DMSO (0.5 mL), and filtered through a 0.45 m PTFE filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[5-cyclopropyl-4-(2-methylphenoxy)-6-(o-tolyl)pyrimidin-2-yl]-1-methyl-pyrazole-4-sulfonamide (12.0 mg, 53%) as a white solid. ESI-MS m/z calc. 475.16782, found 476.21 (M+1)⁺; Retention time: 1.83 minutes (LC method A).

### Example 294: Preparation of Compound 1071

### Step 1: N-[4-chloro-5-cyclopropyl-6-(o-tolyl)pyrimidin-2-yl]-1-methyl-pyrazole-4-sulfonamide

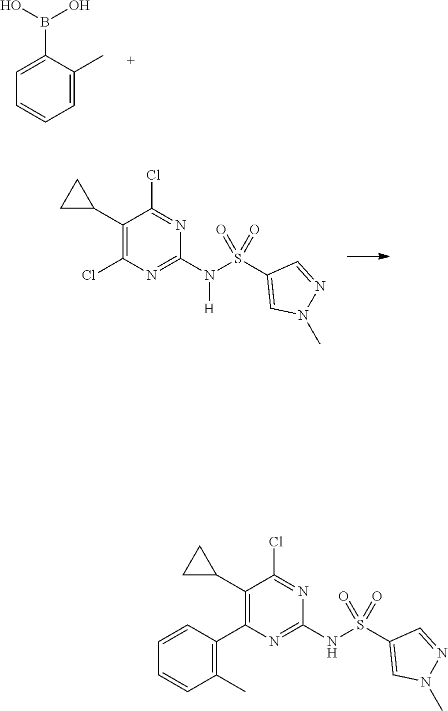

A heterogeneous mixture of bis(triphenylphosphine)palladium(II) dichloride (approximately 11.81 mg, 0.01683 mmol), o-tolylboronic acid (approximately 76.27 mg, 0.5610 mmol), N-(4,6-dichloro-5-cyclopropyl-pyrimidin-2-yl)-1-methyl-pyrazole-4-sulfonamide (195.3 mg, 0.561 mmol) and potassium carbonate (approximately 232.6 mg, 1.683 mmol) in dioxane (1.870 mL) and water (374.1 μL) was heated in a sealed vial to 90° C. for 16 hours. The mixture was acidified with acetic acid (approximately 673.8 mg, 638.1 μL, 11.22 mmol), diluted with DMSO (1.0 mL), and filtered through a 0.45 m syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-chloro-5-cyclopropyl-6-(o-tolyl)pyrimidin-2-yl]-1-methyl-pyrazole-4-sulfonamide (21 mg, 9%) as a white solid. ESI-MS m/z calc. 403.08698, found 404.15 (M+1)⁺; Retention time: 0.65 minutes (LC method D).

### Step 2: N-[5-cyclopropyl-4-[3-(4-methylpiperazin-1-yl)phenoxy]-6-(o-tolyl)pyrimidin-2-yl]-1-methyl-pyrazole-4-sulfonamide

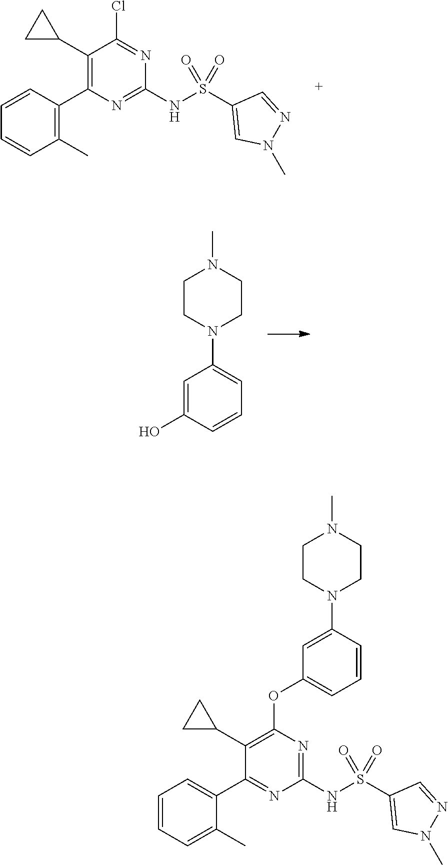 [SMILES: Cc1ccccc1-c1nc(NS(=O)(=O)c2cnn(C)c2)nc(Cl)c1C1CC1 ; CN1CCN(c2cccc(O)c2)CC1 ; Cc1ccccc1-c1nc(NS(=O)(=O)c2cnn(C)c2)nc(Oc2cccc(N3CCN(C)CC3)c2)c1C1CC1]

A heterogeneous mixture of N-[4-chloro-5-cyclopropyl-6-(o-tolyl)pyrimidin-2-yl]-1 -methyl-pyrazole-4-sulfonamide (10.10 mg, 0.0250 mmol), 3-(4-methylpiperazin-1-yl)phenol, and potassium carbonate in NMP (50.00 μL) was heated in a sealed vial to 115° C. for 16 hours. The solution was acidified with acetic acid. The sample was purified by reverse phase HPLC (Waters Sunfire C₁₈ column (100×50 mm, 10 m particle size), gradient: 1-99% acetonitrile in water (5 mM HCl) over 15.0 minutes) to afford N-[5-cyclopropyl-4-[3-(4-methylpiperazin-1-yl)phenoxy]-6-(o-tolyl)pyrimidin-2-yl]-1-methyl-pyrazole-4-sulfonamide (hydrochloride salt) as a white solid (6.8 mg). ESI-MS m/z calc. 559.2366, found 560.3 (M+1)⁺; Retention time: 1.38 minutes; LC method A.

### Example 296: Preparation of Compound 1072

### Step 1: N-[4-[2-chloro-3-(4-methylpiperazin-1-yl)phenoxy]-5-cyclopropyl-6-(o-tolyl)pyrimidin-2-yl]-1-methyl-pyrazole-4-sulfonamide

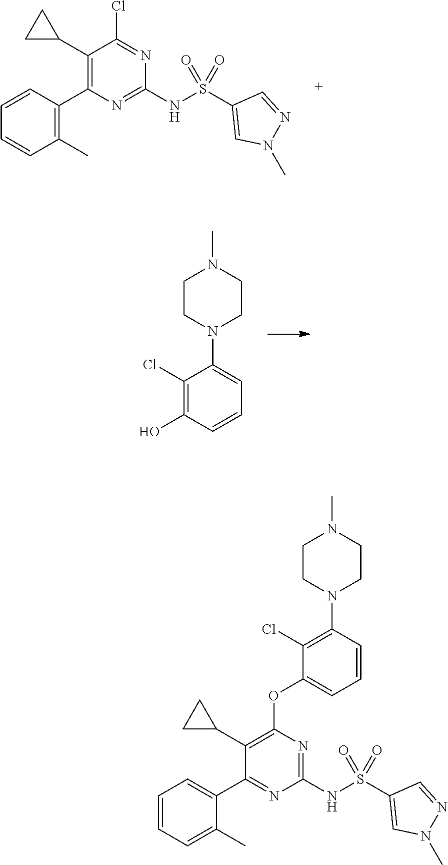 [SMILES: Cc1ccccc1-c1nc(NS(=O)(=O)c2cnn(C)c2)nc(Cl)c1C1CC1 ; CN1CCN(c2cccc(O)c2Cl)CC1 ; Cc1ccccc1-c1nc(NS(=O)(=O)c2cnn(C)c2)nc(Oc2cccc(N3CCN(C)CC3)c2Cl)c1C1CC1]

A heterogeneous solution of N-[4-chloro-5-cyclopropyl-6-(o-tolyl)pyrimidin-2-yl]-1 -methyl-pyrazole-4-sulfonamide (10.10 mg, 0.0250 mmol), 2-chloro-3-(4-methylpiperazin-1-yl)phenol, and potassium carbonate inNMP (50.00 μL) was heated in a sealed vial to 115° C. for 16 hours. The solution was acidified with acetic acid. The sample was purified by reverse phase HPLC (Waters Sunfire C₁₈ column (100×50 mm, 10 m particle size), gradient: 1-99% acetonitrile in water (5 mM HCl) over 15.0 minutes) to afford N-[4-[2-chloro-3-(4-methylpiperazin-1-yl)phenoxy]-5-cyclopropyl-6-(o-tolyl)pyrimidin-2-yl]-1-methyl-pyrazole-4-sulfonamide (hydrochloride salt) as a white solid (8 mg). ESI-MS m/z calc. 593.1976, found 594.29 (M+1)⁺; Retention time: 1.45 minutes; LC method A.

### Example 297: Preparation of Compound 1073

### Step 1: N-[4-chloro-5-cyclopropyl-6-[4-(1-methyl-4-piperidyl)phenoxy]pyrimidin-2-yl]-1-methyl-pyrazole-4-sulfonamide

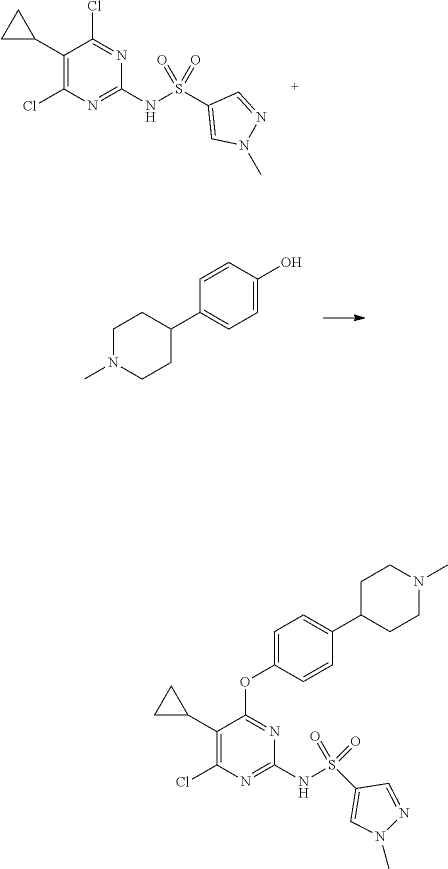 [SMILES: Cn1cc(S(=O)(=O)Nc2nc(Cl)c(C3CC3)c(Cl)n2)cn1 ; CN1CCC(c2ccc(O)cc2)CC1 ; CN1CCC(c2ccc(Oc3nc(NS(=O)(=O)c4cnn(C)c4)nc(Cl)c3C3CC3)cc2)CC1]

A heterogeneous solution of N-(4,6-dichloro-5-cyclopropyl-pyrimidin-2-yl)-1-methyl-pyrazole-4-sulfonamide (approximately 306.1 mg, 0.8790 mmol), 4-(1-methyl-4-piperidyl)phenol (acetate salt) (approximately 220.9 mg, 0.8790 mmol), and potassium carbonate (approximately 485.9 mg, 3.516 mmol) in NMP (1.758 mL) was heated in a sealed vial to 110° C. for 16 hours. The reaction mixture was acidified with acetic acid (1 mL, 17.58 mmol), diluted with water (0.20 mL), and filtered through a 0.20 m filter syringe. The sample was purified by reverse phase HPLC (Waters Sunfire C₁₈ column (100×50 mm, 10 m particle size), gradient: 1-99% acetonitrile in water (5 mM HCl) over 15.0 minutes) to afford N-[4-chloro-5-cyclopropyl-6-[4-(1-methyl-4-piperidyl)phenoxy]pyrimidin-2-yl]-1-methyl-pyrazole-4-sulfonamide (Trifluoroacetate salt) (110 mg, 20%) as a gel. ESI-MS m/z calc. 502.1554, found 503.27 (M+1)⁺; Retention time: 0.53 minutes (LC method D).

### Step 2: N-[5-Cyclopropyl-4-(2,6-dimethylphenyl)-6-[4-(1-methyl-4-piperidyl)phenoxy]pyrimidin-2-yl]-1-methyl-pyrazole-4-sulfonamide

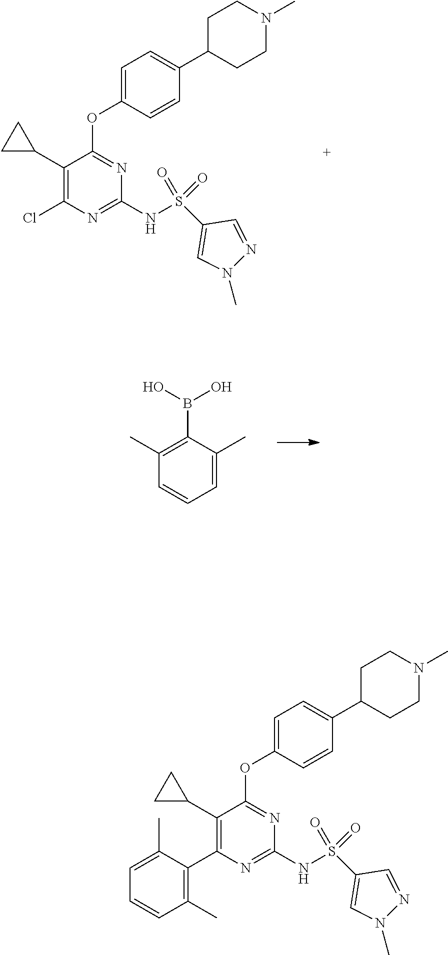

A heterogeneous solution of N-[4-chloro-5-cyclopropyl-6-[4-(1-methyl-4-piperidyl)phenoxy]pyrimidin-2-yl]-1-methyl-pyrazole-4-sulfonamide (trifluoroacetate salt) (20.0 mg, 0.03241 mmol), (2,6-dimethylphenyl)boronic acid (9.7 mg, 0.06467 mmol), tetrakis(triphenylphosphine)palladium(0) (7.5 mg, 0.006490 mmol) and potassium carbonate (17.9 mg, 0.1295 mmol) in dioxane (135 μL) and water (30 μL) was microwaved in a sealed vial to 125° C. for 30 minutes. The reaction mixture was acidified with acetic acid (60.0 μL, 1.055 mmol), diluted with DMSO (0.5 mL), and filtered through a 0.45 m PTFE filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[5-cyclopropyl-4-(2,6-dimethylphenyl)-6-[4-(1-methyl-4-piperidyl)phenoxy]pyrimidin-2-yl]-1 -methyl-pyrazole-4-sulfonamide (hydrochloride salt) (5.3 mg, 27%) as a white solid. ESI-MS m z calc. 572.25696, found 573.5 (M+1)⁺; Retention time: 1.31 minutes (LC method A).

### Example 298: Preparation of Compound 1074

### Step 1: N-[4-Chloro-5-cyclopropyl-6-[4-(1-methyl-4-piperidyl)phenoxy]pyrimidin-2-yl]-1-methyl-pyrazole-4-sulfonamide

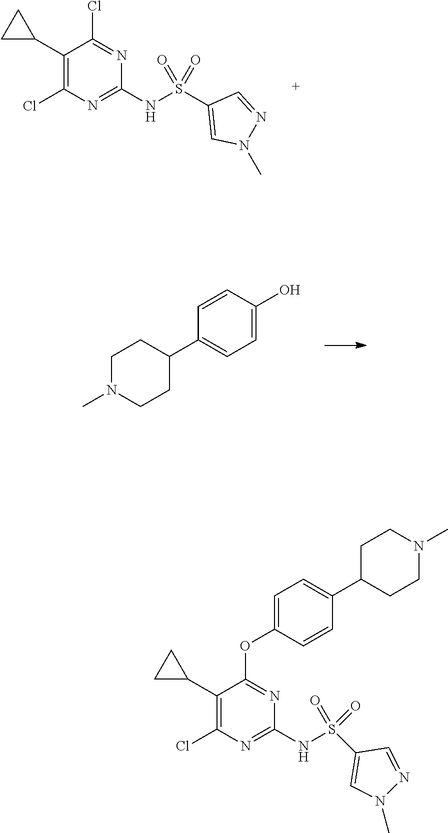 [SMILES: Cn1cc(S(=O)(=O)Nc2nc(Cl)c(C3CC3)c(Cl)n2)cn1 ; CN1CCC(c2ccc(O)cc2)CC1 ; CN1CCC(c2ccc(Oc3nc(NS(=O)(=O)c4cnn(C)c4)nc(Cl)c3C3CC3)cc2)CC1]

A heterogeneous solution of N-(4,6-dichloro-5-cyclopropyl-pyrimidin-2-yl)-1-methyl-pyrazole-4-sulfonamide (approximately 306.1 mg, 0.8790 mmol), 4-(1-methyl-4-piperidyl)phenol (acetate salt) (approximately 220.9 mg, 0.8790 mmol), and potassium carbonate (approximately 485.9 mg, 3.516 mmol) in NMP (1.758 mL) was heated in a sealed vial to 110° C. for 16 hours. The reaction mixture was acidified with acetic acid (1 mL, 17.58 mmol), diluted with water (0.20 mL), and filtered through a 0.20 m filter syringe. The sample was purified by reverse phase HPLC (Waters Sunfire C₁₈ column (100×50 mm, 10 m particle size), gradient: 1-99% acetonitrile in water (5 mM HCl) over 15.0 minutes) to afford N-[4-chloro-5-cyclopropyl-6-[4-(1-methyl-4-piperidyl)phenoxy]pyrimidin-2-yl]-1-methyl-pyrazole-4-sulfonamide (Trifluoroacetate salt) (110 mg, 20%) as a gel. ESI-MS m/z calc. 502.1554, found 503.27 (M+1)⁺; Retention time: 0.53 minutes (LC method D).

### Step 2: N-[5-cyclopropyl-4-(2-isobutylphenyl)-6-[4-(1-methyl-4-piperidyl)phenoxy]pyrimidin-2-yl]-1-methyl-pyrazole-4-sulfonamide

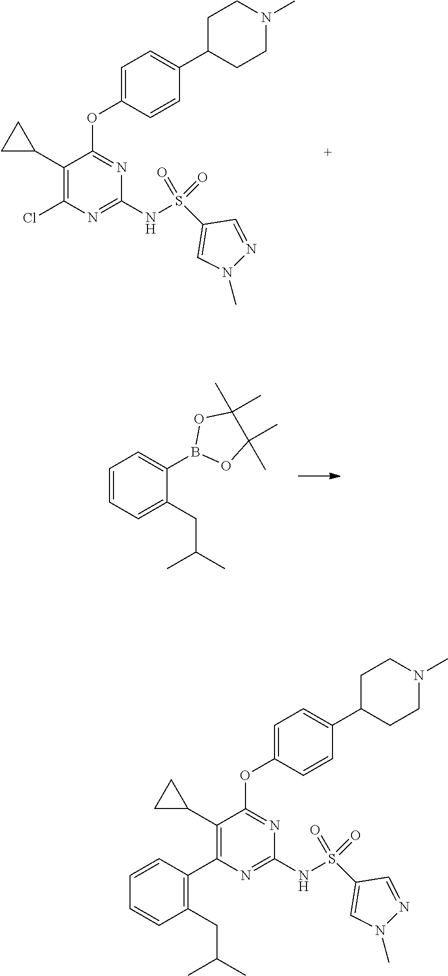 [SMILES: CN1CCC(c2ccc(Oc3nc(NS(=O)(=O)c4cnn(C)c4)nc(Cl)c3C3CC3)cc2)CC1 ; CC(C)Cc1ccccc1B1OC(C)(C)C(C)(C)O1 ; CC(C)Cc1ccccc1-c1nc(NS(=O)(=O)c2cnn(C)c2)nc(Oc2ccc(C3CCN(C)CC3)cc2)c1C1CC1]

A heterogeneous solution of N-[4-chloro-5-cyclopropyl-6-[4-(1-methyl-4-piperidyl)phenoxy]pyrimidin-2-yl]-1-methyl-pyrazole-4-sulfonamide (Trifluoroacetate salt) (20 mg, 0.03241 mmol), 2-(2-isobutylphenyl)-4,4,5,5-tetramethyl-1,3,2-dioxaborolane (16.9 mg, 0.06496 mmol), potassium carbonate (17.9 mg, 0.1295 mmol) and tetrakis(triphenylphosphine)palladium(0) (7.5 mg, 0.006490 mmol) in dioxane (135 μL) and water (30 μL) was microwaved in a sealed vial to 125° C. for 30 minutes. The reaction mixture was acidified with acetic acid (60.0 μL, 1.055 mmol), diluted with DMSO (0.5 mL), and filtered through a 0.45 m PTFE filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[5-cyclopropyl-4-(2-isobutylphenyl)-6-[4-(1-methyl-4-piperidyl)phenoxy]pyrimidin-2-yl]-1-methyl-pyrazole-4-sulfonamide (hydrochloride salt) (5.1 mg, 25%) as a white solid. ESI-MS m/z calc. 600.28827, found 601.55 (M+1)⁺; Retention time: 1.51 minutes (LC method A).

### Example 299: Preparation of Compound 1075

### Step 1: N-[5-Cyclopropyl-4-(2,6-dimethylphenyl)-6-(2-methylphenoxy)pyrimidin-2-yl]-1-methyl-pyrazole-4-sulfonamide

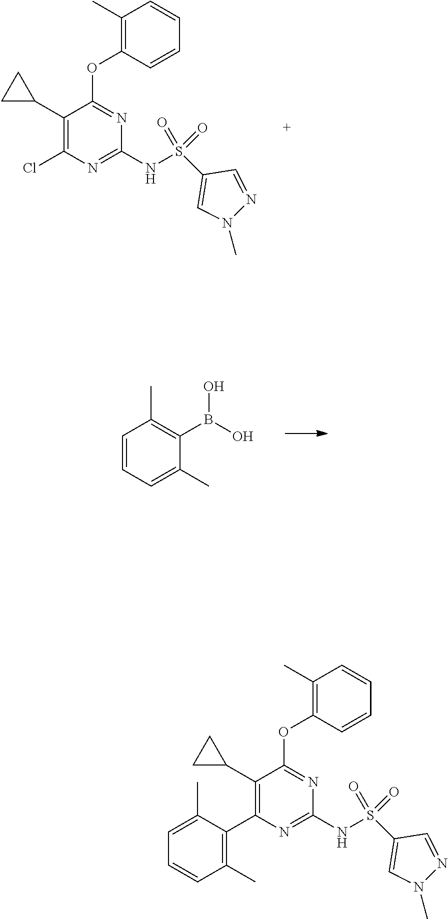 [SMILES: Cc1ccccc1Oc1nc(NS(=O)(=O)c2cnn(C)c2)nc(Cl)c1C1CC1 ; Cc1cccc(C)c1B(O)O ; Cc1ccccc1Oc1nc(NS(=O)(=O)c2cnn(C)c2)nc(-c2c(C)cccc2C)c1C1CC1]

A heterogeneous solution of (2,6-dimethylphenyl)boronic acid (14.3 mg, 0.09534 mmol), N-[4-chloro-5-cyclopropyl-6-(2-methylphenoxy)pyrimidin-2-yl]-1-methyl-pyrazole-4-sulfonamide (20.0 mg, 0.04763 mmol), tetrakis(triphenylphosphine)palladium(0) (11.0 mg, 0.009519 mmol), and potassium carbonate (19.8 mg, 0.1433 mmol) in dioxane (160 μL) and water (30 μL) was microwaved in a sealed vial to 125° C. for 30 minutes. The reaction mixture was acidified with acetic acid (60.0 μL, 1.055 mmol), diluted with DMSO (0.5 mL), and filtered through a 0.45 m PTFE filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[5-cyclopropyl-4-(2,6-dimethylphenyl)-6-(2-methylphenoxy)pyrimidin-2-yl]-1-methyl-pyrazole-4-sulfonamide (8.2 mg, 33%) as a white solid. ESI-MS m/z calc. 489.18347, found 490.3 (M+1)⁺; Retention time: 1.89 minutes (LC method A).

### Example 300: Preparation of Compound 1076

### Step 1: N-[5-cyclopropyl-4-(2-isobutylphenyl)-6-(2-methylphenoxy)pyrimidin-2-yl]-1-methyl-pyrazole-4-sulfonamide

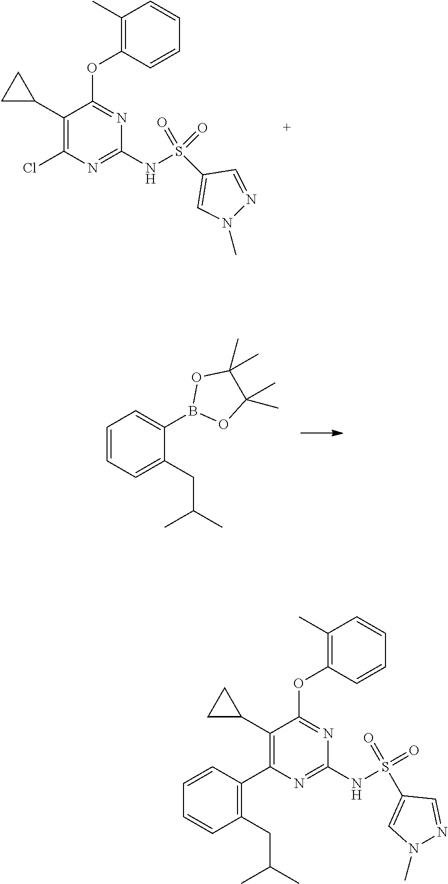 [SMILES: Cc1ccccc1Oc1nc(NS(=O)(=O)c2cnn(C)c2)nc(Cl)c1C1CC1 ; CC(C)Cc1ccccc1B1OC(C)(C)C(C)(C)O1 ; Cc1ccccc1Oc1nc(NS(=O)(=O)c2cnn(C)c2)nc(-c2ccccc2CC(C)C)c1C1CC1]

A heterogeneous solution of N-[4-chloro-5-cyclopropyl-6-(2-methylphenoxy)pyrimidin-2-yl]-1-methyl-pyrazole-4-sulfonamide (20.00 mg, 0.04763 mmol), tetrakis(triphenylphosphine)palladium(0) (11 mg, 0.009519 mmol), potassium carbonate (19.8 mg, 0.1433 mmol), 2-(2-isobutylphenyl)-4,4,5,5-tetramethyl-1,3,2-dioxaborolane (12.4 mg, 0.04766 mmol) in dioxane (160 μL) and water (30 μL) was microwaved in a sealed vial to 125° C. for 30 minutes. The reaction mixture was acidified with acetic acid (60 μL, 1.055 mmol), diluted with DMSO (0.5 mL), and filtered through a 0.45 m PTFE filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[5-cyclopropyl-4-(2-isobutylphenyl)-6-(2-methylphenoxy)pyrimidin-2-yl]-1-methyl-pyrazole-4-sulfonamide (5.9 mg, 24%) as a white solid. ESI-MS m/z calc. 517.2148, found 518.31 (M+1)⁺; Retention time: 2.11 minutes (LC method A).

### Example 301: Preparation of Compound 1077

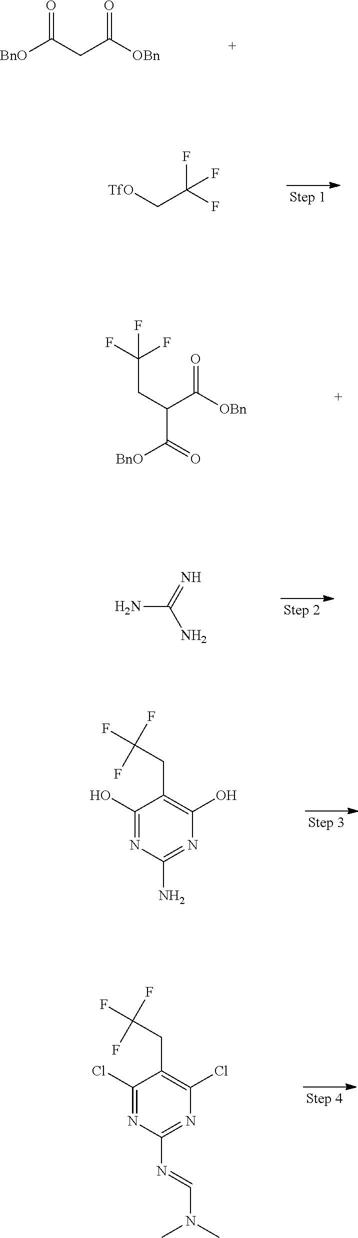

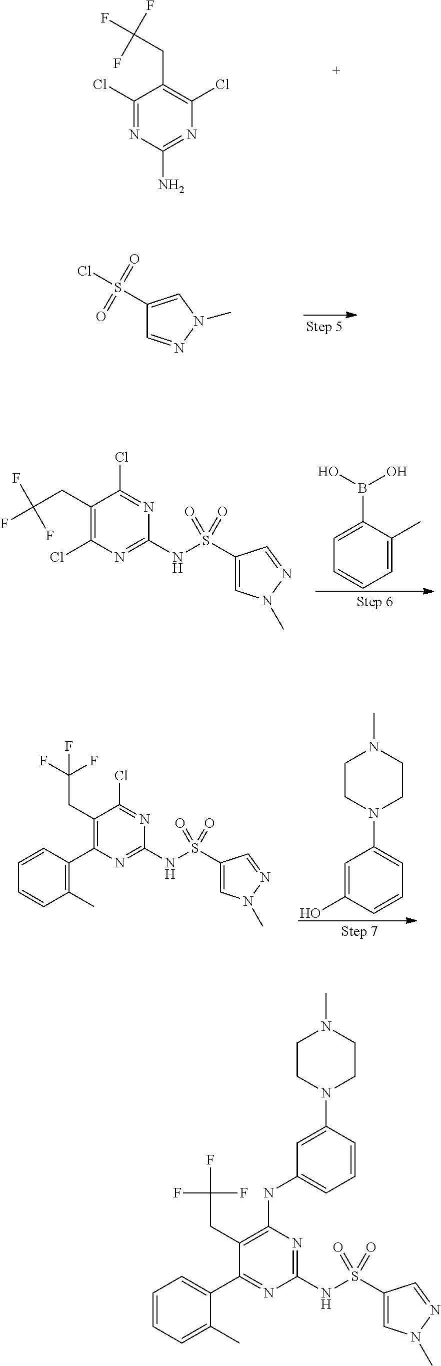

### Step 1: Dibenzyl 2-(2,2,2-trifluoroethyl)propanedioate

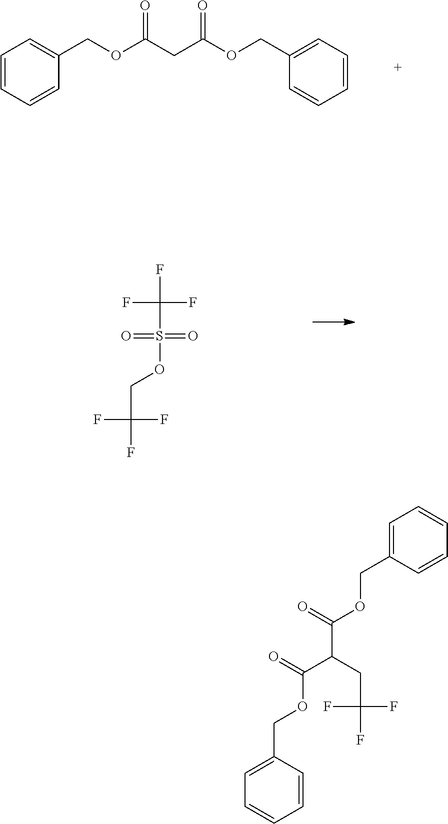 [SMILES: O=C(CC(=O)OCc1ccccc1)OCc1ccccc1 ; O=S(=O)(OCC(F)(F)F)C(F)(F)F ; O=C(OCc1ccccc1)C(CC(F)(F)F)C(=O)OCc1ccccc1]

Dibenzyl propanedioate (352.5 mg, 0.31 mL, 1.240 mmol) was added to a suspension of 60% sodium hydride (95 mg, 2.375 mmol) in tetrahydrofuran (5 mL) at room temperature and the mixture was stirred for 30 minutes to give a clear solution. 2,2,2-trifluoroethyl trifluoromethanesulfonate (257.8 mg, 0.16 mL, 1.111 mmol) was added and the reaction mixture was heated at 50° C. for 48 hours. Once cooled to room temperature, the reaction mixture was mixed with another reaction run on the same scale and concentrated under reduced pressure and the residue was diluted in diethyl ether. Aqueous saturated ammonium chloride was added followed by 1 N HCl and the mixture was extracted with diethyl ether (3×15 mL). Organic layers were combined, dried over magnesium sulfate and concentrated under reduced pressure. The residue was purified by silica gel chromatography using 0% to 15% of EtOAc in heptanes to afford dibenzyl 2-(2,2,2-trifluoroethyl)propanedioate (454 mg, 56%) as a clear oil. ¹H NMR (300 MHz, CDCl₃) δ ppm 2.85 (qd, J=10.4, 6.9 Hz, 2H), 3.76 (t, J=6.9 Hz, 1H), 5.16 (d, J=1.2 Hz, 4H), 7.21-7.40 (m, 10H). ¹⁹F NMR (282 MHz, CDCl₃) δ ppm −66.0-−65.7 (m, 3F). ESI-MS m/z calc. 366.331, found 389.1 (M+Na)*; Retention time: 2.37 minutes (LC method A).

### Step 2: 2-Amino-5-(2,2,2-trifluoroethyl)pyrimidine-4,6-diol

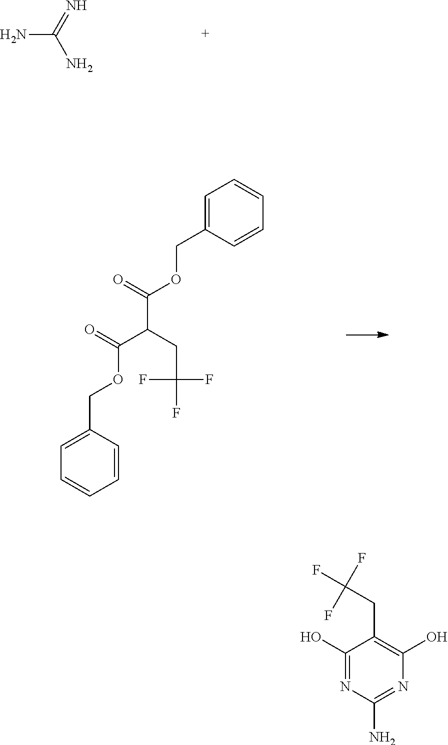

Solid chunks of sodium metal (95 mg, 4.132 mmol) were added gradually and carefully to ethanol (7 mL) and the mixture was stirred until completely dissolved. Once cooled back to room temperature guanidine hydrochloride (150 mg, 1.570 mmol) and dibenzyl 2-(2,2,2-trifluoroethyl)propanedioate (450 mg, 1.228 mmol) were successively added and the reaction was stirred in an oil bath set at 80° C. overnight. Once cooled to room temperature, the crude mixture was concentrated under reduced pressure to remove most of the ethanol. Water (10 mL) was added, the resulting solution was cooled in an ice bath and acidified to pH of 1-2 using 6 N HCl. The solids were filtered and washed with water (2×2 mL) and dried under high vacuum to afford 2-amino-5-(2,2,2-trifluoroethyl)pyrimidine-4,6-diol (174 mg, 68%) as a white solid. ¹H NMR (300 MHz, DMSO-d₆) δ ppm 2.99 (q, J=11.3 Hz, 2H), 6.71 (br. s., 2H), 10.56 (br. s., 2H). ¹⁹F NMR (282 MHz, DMSO-d₆) δ ppm −64.4 (t, J=9.2 Hz, 3F). ESI-MS m/z calc. 209.126, found 210.1 (M+1)⁺+; Retention time: 0.46 minutes (LC method C).

### Step 3: N′-[4,6-Dichloro-5-(2,2,2-trifluoroethyl)pyrimidin-2-yl]-N,N-dimethyl-formamidine

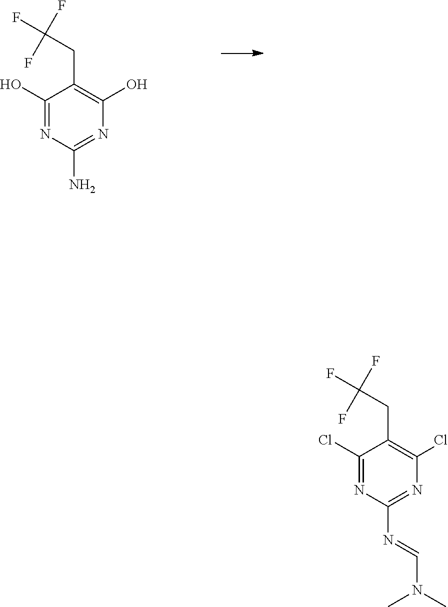 [SMILES: Nc1nc(O)c(CC(F)(F)F)c(O)n1 ; CN(C)/C=N/c1nc(Cl)c(CC(F)(F)F)c(Cl)n1]

Oxalyl chloride (65.25 g, 45 mL, 514.1 mmol) was slowly added to a solution of dimethylformamide (37.60 g, 40 mL, 514.4 mmol) in chloroform (400 mL) and the solution was stirred at room temperature for 30 minutes. 2-Amino-5-(2,2,2-trifluoroethyl)pyrimidine-4,6-diol (10.8 g, 51.64 mmol) was added then the reaction mixture was heated at reflux overnight. Once cooled to room temperature, the reaction mixture was diluted with sodium bicarbonate (500 mL) and stirred vigorously for 15 minutes. Layers were separated and the aqueous layer was extracted with dichloromethane (2×200 mL). Organic layers were combined, dried over sodium sulfate and concentrated under reduced pressure to afford N-[4,6-dichloro-5-(2,2,2-trifluoroethyl)pyrimidin-2-yl]-N,N-dimethyl-formamidine (32.73 g, 210%) as brown oil. The crude material was used for next step without any further purification. ESI-MS m/z calc. 300.01562, found 301.1 (M+1)⁺; Retention time: 1.56 minutes (LC method C).

### Step 4: 4,6-Dichloro-5-(2,2,2-trifluoroethyl)pyrimidin-2-amine

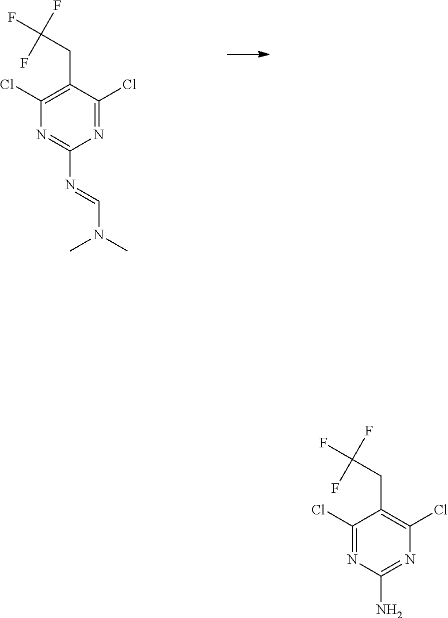 [SMILES: CN(C)/C=N/c1nc(Cl)c(CC(F)(F)F)c(Cl)n1 ; Nc1nc(Cl)c(CC(F)(F)F)c(Cl)n1]

Concentrated hydrochloric acid (27 mL of 12 M, 324.0 mmol) was added to a solution of N-[4,6-dichloro-5-(2,2,2-trifluoroethyl)pyrimidin-2-yl]-N,N-dimethyl-formamidine (15.55 g, 51.64 mmol) in isopropanol (160 mL) and the mixture was stirred at 50° C. for 90 minutes. The solution was concentrated under reduced pressure. The crude material was combined with the crude from another reaction run on 1 g scale and transferred to a 1.0-L separatory funnel with water (about 150 mL) and 5% aqueous sodium bicarbonate. The aqueous layer was then extracted with DCM (4×150 mL). Solids were observed in the organic layer, which were filtered off and washed with additional DCM to afford a first lot of 4,6-dichloro-5-(2,2,2-trifluoroethyl)pyrimidin-2-amine (5.535 g, 44%) as a beige solid. ¹H NMR (300 MHz, DMSO-d₆) ppm 3.68 (q, J=10.4 Hz, 2H), 7.72 (s, 2H). ¹⁹F NMR (282 MHz, DMSO-d₆) ppm −66.6-−61.2 (m, 3F). ESI-MS m/z calc. 244.9734, found 246.0 (M+1)⁺; Retention time: 2.43 minutes (LC method B). The organic layer was then transferred back to a 1.0-L separatory funnel and washed with brine (about 200 mL). The organic layer was dried over sodium sulfate, filtered and concentrated under reduced pressure to a final volume of about 50-75 mL. The solid that crashed out was filtered and washed with additional DCM to afford a second lot of 4,6-dichloro-5-(2,2,2-trifluoroethyl)pyrimidin-2-amine (1.845 g, 14%) as a beige solid. ¹H NMR (300 MHz, DMSO-d₆) δ ppm 3.68 (q, J=10.6 Hz, 2H), 7.72 (s, 2H). ¹⁹F NMR (282 MHz, DMSO-d₆) δ ppm −63.9−-63.4 (m, 3F). ESI-MS m/z calc. 244.9734, found 246.1 (M+1)⁺; Retention time: 2.43 minutes (LC method B).

### Step 5: N-[4,6-Dichloro-5-(2,2,2-trifluoroethyl)pyrimidin-2-yl]-1-methyl-pyrazole-4-sulfonamide

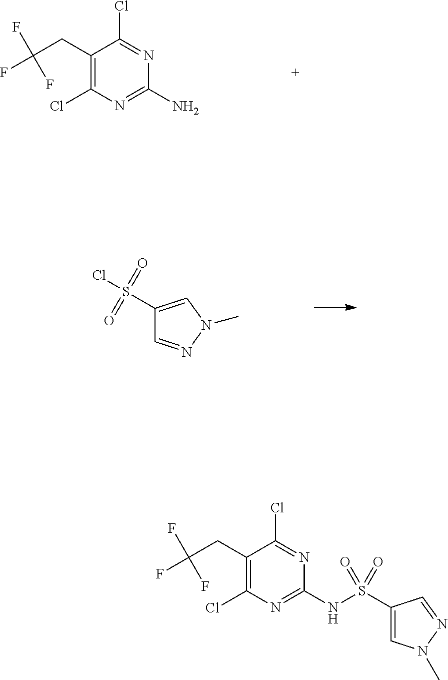

To a solution of 4,6-dichloro-5-(2,2,2-trifluoroethyl)pyrimidin-2-amine (2.00 g, 8.130 mmol) in DMF (32.5 mL) at 0° C. was added sodium hydride (1.30 g of 60% w/w, 32.50 mmol). The reaction was allowed to warm to 23° C. over 15 minutes. The solution was cooled to 0° C. and 1-methylpyrazole-4-sulfonyl chloride (2.94 g, 16.28 mmol) was added. The reaction mixture was allowed to warm to 23° C. over 15 minutes. The solution was cooled to 0° C., acidified with acetic acid (6.9 mL, 121.3 mmol), and partitioned between ethyl acetate and water. The organic layer was separated, and the aqueous layer was further extracted with ethyl acetate (4×). The combined organics were washed with brine, dried over magnesium sulfate, filtered, and concentrated in vacuo. The crude solid was triturated twice with acetone to afford N-[4,6-dichloro-5-(2,2,2-trifluoroethyl)pyrimidin-2-yl]-1-methyl-pyrazole-4-sulfonamide (2.23 g, 70%) as a white solid. ESI-MS m/z calc. 388.97278, found 390.0 (M+1)⁺; Retention time: 0.53 minutes (LC method D).

### Step 6: N-[4-chloro-6-(o-tolyl)-5-(2,2,2-trifluoroethyl)pyrimidin-2-yl]-1-methyl-pyrazole-4-sulfonamide

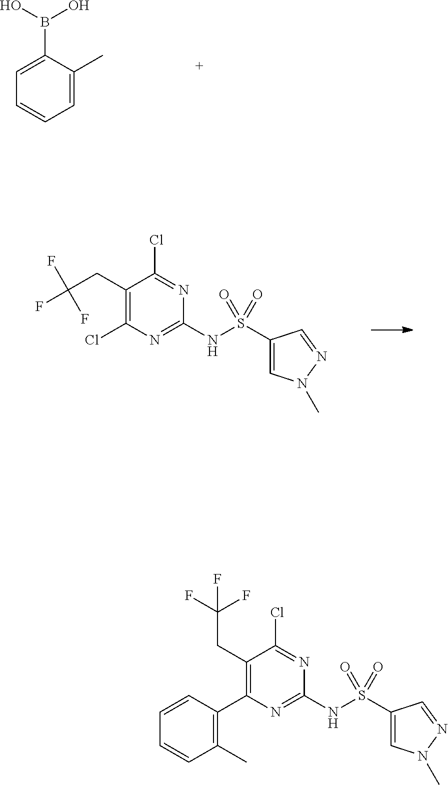

A heterogeneous solution of o-tolylboronic acid (approximately 76.33 mg, 0.5614 mmol), bis(triphenylphosphine)palladium(II) dichloride(approximately 11.82 mg, 0.01684 mmol), N-[4,6-dichloro-5-(2,2,2-trifluoroethyl)pyrimidin-2-yl]-1-methyl-pyrazole-4-sulfonamide (219.0 mg, 0.5614 mmol), and potassium carbonate (approximately 232.7 mg, 1.684 mmol) in dioxane (1.871 mL) and water (374.4 μL) was heated in a sealed vial to 90° C. for 16 hours. The solution was acidified with acetic acid (approximately 674.4 mg, 638.6 μL, 11.23 mmol), diluted with DMSO (1.0 mL), and filtered through a 0.45 m syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-chloro-6-(o-tolyl)-5-(2,2,2-trifluoroethyl)pyrimidin-2-yl]-1-methyl-pyrazole-4-sulfonamide (30 mg, 12%) as a white solid. ESI-MS m/z calc. 445.05872, found 446.14 (M+1)⁺; Retention time: 0.64 minutes (LC method D).

### Step 7: 1-Methyl-N-[4-[3-(4-methylpiperazin-1-yl)phenoxy]-6-(o-tolyl)-5-(2,2,2-trifluoroethyl)pyrimidin-2-yl]pyrazole-4-sulfonamide

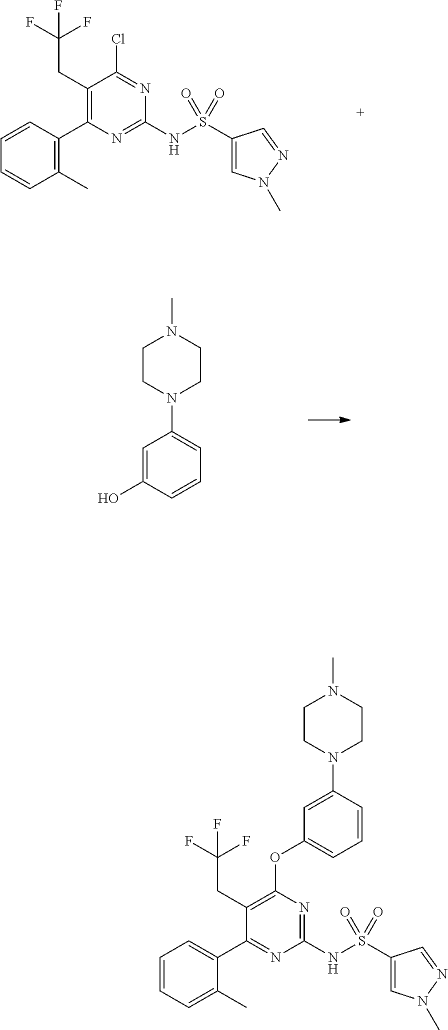 [SMILES: Cc1ccccc1-c1nc(NS(=O)(=O)c2cnn(C)c2)nc(Cl)c1CC(F)(F)F ; CN1CCN(c2cccc(O)c2)CC1 ; Cc1ccccc1-c1nc(NS(=O)(=O)c2cnn(C)c2)nc(Oc2cccc(N3CCN(C)CC3)c2)c1CC(F)(F)F]

A heterogeneous solution of N-[4-chloro-6-(o-tolyl)-5-(2,2,2-trifluoroethyl)pyrimidin-2-yl]-1-methyl-pyrazole-4-sulfonamide (11.15 mg, 0.0250 mmol), 3-(4-methylpiperazin-1-yl)phenol, and potassium carbonate in NMP (50.00 μL) was heated in a sealed vial to 115° C. for 16 hours. The solution was acidified with acetic acid. The sample was purified by reverse phase HPLC (Waters Sunfire C₁₈ column (100×50 mm, 10 m particle size), gradient: 1-99% acetonitrile in water (5 mM HCl) over 15.0 minutes) to afford 1-methyl-N-[4-[3-(4-methylpiperazin-1-yl)phenoxy]-6-(o-tolyl)-5-(2,2,2-trifluoroethyl)pyrimidin-2-yl]pyrazole-4-sulfonamide (hydrochloride salt) as a white solid. ESI-MS m/z calc. 601.2083, found 602.25 (M+1)⁺; Retention time: 1.41 minutes (LC method A).

### Example 302: Preparation of Compound 1078

### Step 1: 1-Methyl-N-[4-[4-(1-methyl-4-piperidyl)phenoxy]-6-phenyl-5-(2,2,2-trifluoroethyl)pyrimidin-2-yl]pyrazole-4-sulfonamide

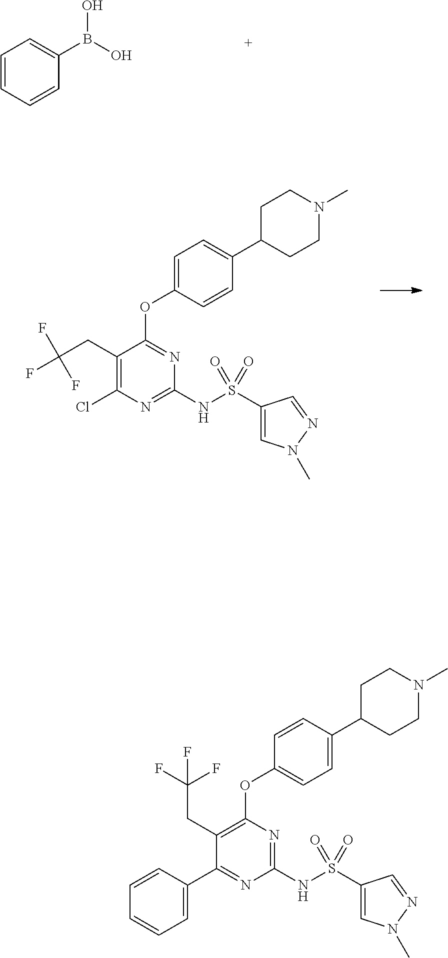 [SMILES: OB(O)c1ccccc1 ; CN1CCC(c2ccc(Oc3nc(NS(=O)(=O)c4cnn(C)c4)nc(Cl)c3CC(F)(F)F)cc2)CC1 ; CN1CCC(c2ccc(Oc3nc(NS(=O)(=O)c4cnn(C)c4)nc(-c4ccccc4)c3CC(F)(F)F)cc2)CC1]

A heterogeneous solution of phenylboronic acid (approximately 30.64 mg, 0.2513 mmol), N-[4-chloro-6-[4-(1-methyl-4-piperidyl)phenoxy]-5-(2,2,2-trifluoroethyl)pyrimidin-2-yl]-1-methyl-pyrazole-4-sulfonamide (75 mg, 0.1005 mmol), tetrakis(triphenylphosphine)palladium(0) (approximately 23.23 mg, 0.02010 mmol), and potassium carbonate (approximately 69.45 mg, 0.5025 mmol) in dioxane (418.7 μL) and water (83.77 μL) was microwaved in a sealed vial to 120° C. for 30 minutes. The crude solution was concentrated to ⅓ the volume by blowing a steady stream of air over the surface. The solution was acidified with acetic acid (approximately 120.7 mg, 114.3 μL, 2.010 mmol) and diluted with DMSO (2.0 mL). The sample was purified by reverse phase HPLC (Phenomenex Luna C₁₈ column (75×30 mm, 5 m particle size), gradient: 1-99% acetonitrile in water (5 mM HCl) over 15.0 minutes) to afford the title compound as a white solid. 1-Methyl-N-[4-[4-(1-methyl-4-piperidyl)phenoxy]-6-phenyl-5-(2,2,2-trifluoroethyl)pyrimidin-2-yl]pyrazole-4-sulfonamide (1 mg, 2%). ESI-MS m/z calc. 586.1974, found 587.29 (M+1)⁺; Retention time: 1.28 minutes; LC method A.

### Example 303: Preparation of Compound 1079

### Step 1: N-[4-(2,4-Dimethylphenyl)-6-[4-(1-methyl-4-piperidyl)phenoxy]-5-(2,2,2-trifluoroethyl)pyrimidin-2-yl]-1-methyl-pyrazole-4-sulfonamide

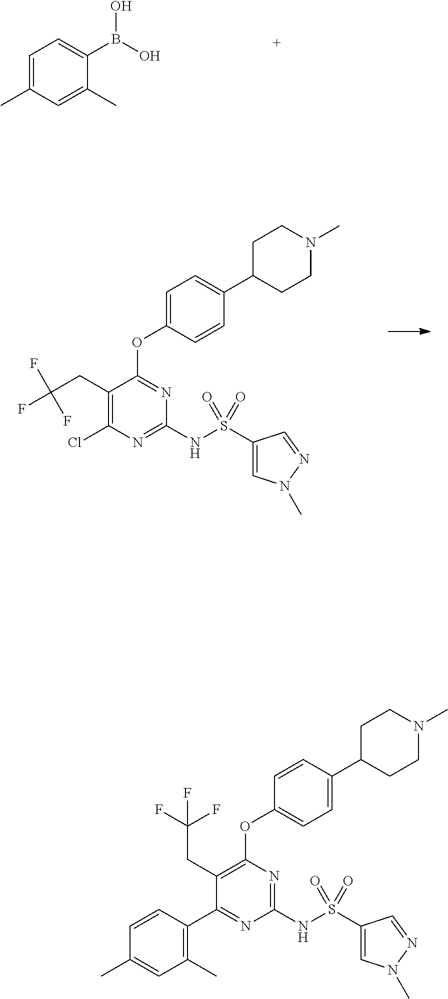

A heterogeneous solution of (2,4-dimethylphenyl)boronic acid (approximately 37.69 mg, 0.2513 mmol), N-[4-chloro-6-[4-(1-methyl-4-piperidyl)phenoxy]-5-(2,2,2-trifluoroethyl)pyrimidin-2-yl]-1-methyl-pyrazole-4-sulfonamide (75 mg, 0.1005 mmol), tetrakis(triphenylphosphine)palladium(0) (approximately 23.23 mg, 0.02010 mmol), and potassium carbonate (approximately 69.45 mg, 0.5025 mmol) in dioxane (418.7 μL) and water (83.77 μL) was microwaved in a sealed vial to 120° C. for 30 minutes. The crude solution was concentrated to ⅓ the volume by blowing a steady stream of air over the surface. The solution was acidified with acetic acid (approximately 120.7 mg, 114.3 μL, 2.010 mmol) and diluted with DMSO (2.0 mL). The sample was purified by reverse phase HPLC (Phenomenex Luna C₁₈ column (75×30 mm, 5 m particle size), gradient: 1-99% acetonitrile in water (5 mM HCl) over 15.0 minutes) to afford the title compound as a white solid. N-[4-(2,4-dimethylphenyl)-6-[4-(1-methyl-4-piperidyl)phenoxy]-5-(2,2,2-trifluoroethyl)pyrimidin-2-yl]-1-methyl-pyrazole-4-sulfonamide (2.3 mg, 4%). ESI-MS m/z calc. 614.2287, found 615.45 (M+1)⁺; Retention time: 1.41 minutes; LC method A.

### Example 304: Preparation of Compound 1080

### Step 1: N-[4-[2-chloro-3-(4-methylpiperazin-1-yl)phenoxy]-6-(o-tolyl)-5-(2,2,2-trifluoroethyl)pyrimidin-2-yl]-1-methyl-pyrazole-4-sulfonamide

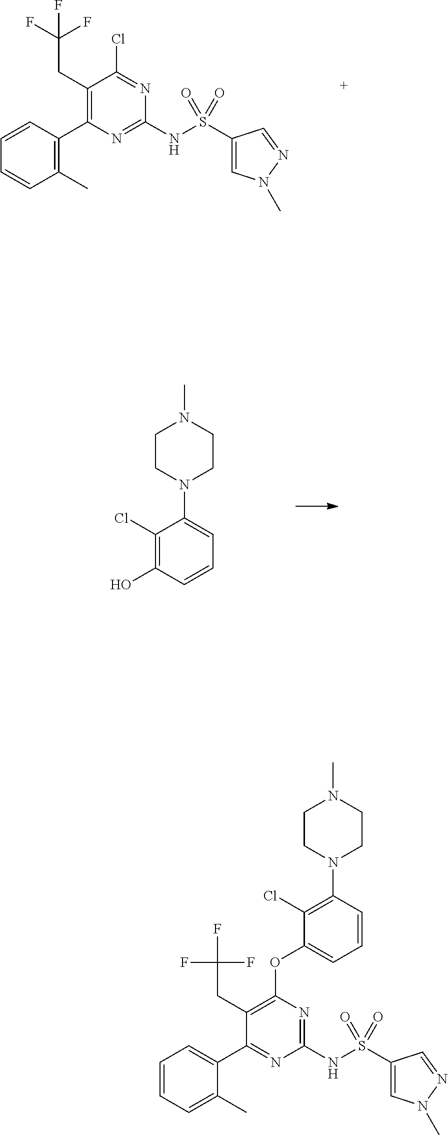 [SMILES: Cc1ccccc1-c1nc(NS(=O)(=O)c2cnn(C)c2)nc(Cl)c1CC(F)(F)F ; CN1CCN(c2cccc(O)c2Cl)CC1 ; Cc1ccccc1-c1nc(NS(=O)(=O)c2cnn(C)c2)nc(Oc2cccc(N3CCN(C)CC3)c2Cl)c1CC(F)(F)F]

A heterogeneous solution of N-[4-chloro-6-(o-tolyl)-5-(2,2,2-trifluoroethyl)pyrimidin-2-yl]-1-methyl-pyrazole-4-sulfonamide (11.15 mg, 0.0250 mmol), 2-chloro-3-(4-methylpiperazin-1-yl)phenol, and potassium carbonate in NMP (50.00 μL) was heated in a sealed vial to 115° C. for 16 hours. The solution was acidified with acetic acid. The sample was purified by reverse phase HPLC (Waters Sunfire C₁₈ column (100×50 mm, 10 m particle size), gradient: 1-99% acetonitrile in water (5 mM HCl) over 15.0 minutes) to afford N-[4-[2-chloro-3-(4-methylpiperazin-1-yl)phenoxy]-6-(o-tolyl)-5-(2,2,2-trifluoroethyl)pyrimidin-2-yl]-1-methyl-pyrazole-4-sulfonamide (hydrochloride salt) as a white solid. ESI-MS m/z calc. 635.1693, found 636.24 (M+1)⁺; Retention time: 1.49 minutes; LC method A.

### Example 305: Preparation of Compound 1081

### Step 1: 1-Methyl-N-[4-[4-(1-methyl-4-piperidyl)phenoxy]-6-(o-tolyl)-5-(2,2,2-trifluoroethyl)pyrimidin-2-yl]pyrazole-4-sulfonamide

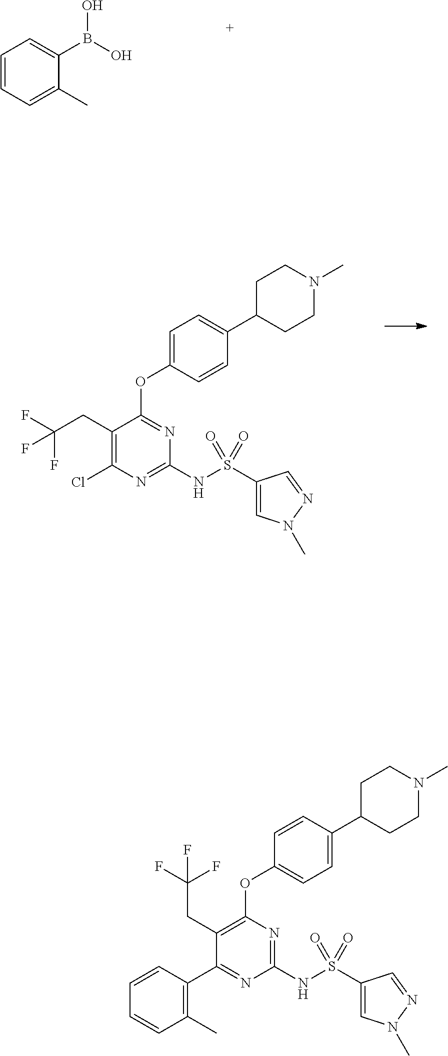

A heterogeneous solution of o-tolylboronic acid (approximately 34.17 mg, 0.2513 mmol), N-[4-chloro-6-[4-(1-methyl-4-piperidyl)phenoxy]-5-(2,2,2-trifluoroethyl)pyrimidin-2-yl]-1-methyl-pyrazole-4-sulfonamide (75 mg, 0.1005 mmol), tetrakis(triphenylphosphine)palladium(0) (approximately 23.23 mg, 0.02010 mmol), and potassium carbonate (approximately 69.45 mg, 0.5025 mmol) in dioxane (418.7 μL) and water (83.77 μL) was microwaved in a sealed vial to 120° C. for 30 minutes. The crude solution was concentrated to ⅓ the volume by blowing a steady stream of air over the surface. The solution was acidified with acetic acid (approximately 120.7 mg, 114.3 μL, 2.010 mmol) and diluted with DMSO (2.0 mL). The sample was purified by reverse phase HPLC (Phenomenex Luna C₁₈ column (75×30 mm, 5 m particle size), gradient: 1-99% acetonitrile in water (5 mM HCl) over 15.0 minutes) to afford the title compound as a white solid. 1-Methyl-N-[4-[4-(1-methyl-4-piperidyl)phenoxy]-6-(o-tolyl)-5-(2,2,2-trifluoroethyl)pyrimidin-2-yl]pyrazole-4-sulfonamide (7.4 mg, 12%). ESI-MS m/z calc. 600.2131, found 601.33 (M+1)⁺; Retention time: 1.33 minutes; LC method A.

### Example 306: Preparation of Compound 1082

### Step 1: N-[4-chloro-6-[4-(1-methyl-4-piperidyl)phenoxy]-5-(2,2,2-trifluoroethyl)pyrimidin-2-yl]-1-methyl-pyrazole-4-sulfonamide

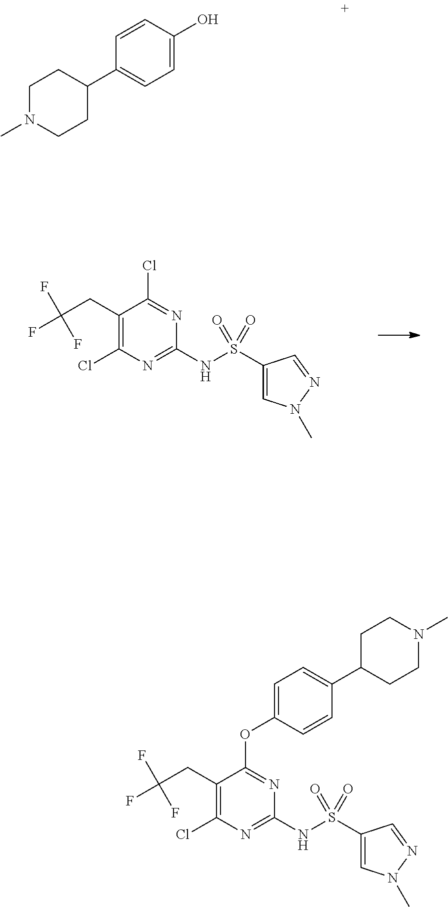 [SMILES: CN1CCC(c2ccc(O)cc2)CC1 ; Cn1cc(S(=O)(=O)Nc2nc(Cl)c(CC(F)(F)F)c(Cl)n2)cn1 ; CN1CCC(c2ccc(Oc3nc(NS(=O)(=O)c4cnn(C)c4)nc(Cl)c3CC(F)(F)F)cc2)CC1]

A heterogeneous solution of N-[4,6-dichloro-5-(2,2,2-trifluoroethyl)pyrimidin-2-yl]-1-methyl-pyrazole-4-sulfonamide (1.5 g, 3.844 mmol), 4-(1-methyl-4-piperidyl)phenol (acetate salt) (966 mg, 3.844 mmol), and potassium carbonate (1.6 g, 11.58 mmol) in NMP (7.6 mL) was heated in a sealed vial to 110° C. for 16 hours. The solution was acidified with acetic acid (3.3 mL, 58.03 mmol) and diluted with water (10 mL). The sample was purified by reverse phase column chromatography (Cis, gradient: 1-99% acetonitrile in water (0.1% formic acid) over 25.0 minutes) to afford N-[4-chloro-6-[4-(1-methyl-4-piperidyl)phenoxy]-5-(2,2,2-trifluoroethyl)pyrimidin-2-yl]-1-methyl-pyrazole-4-sulfonamide (formic acid salt) (1.24 g, 29%) as a white solid. ESI-MS m/z calc. 544.12714, found 545.27 (M+1)⁺; Retention time: 0.49 minutes (LC method D).

### Step 2: N-[4-(2-isobutylphenyl)-6-[4-(1-methyl-4-piperidyl)phenoxy]-5-(2,2,2-trifluoroethyl)pyrimidin-2-yl]-1-methyl-pyrazole-4-sulfonamide

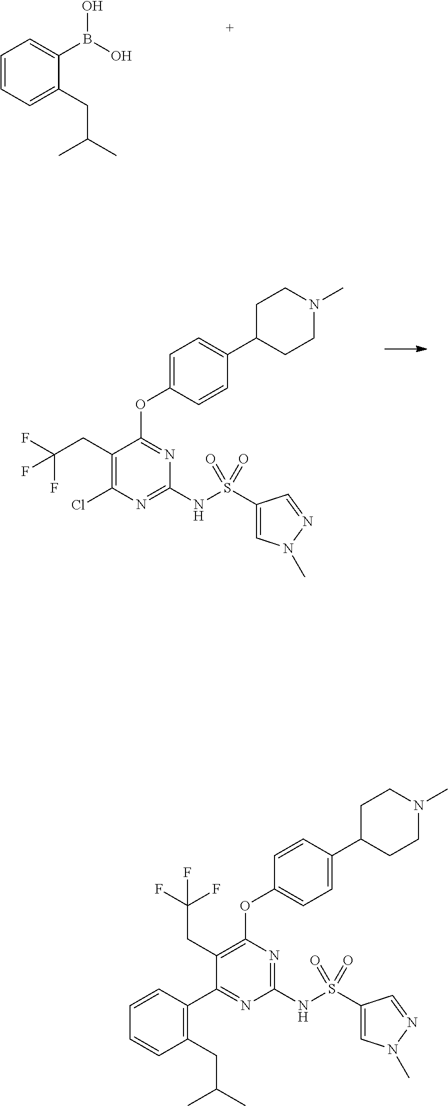 [SMILES: CC(C)Cc1ccccc1B(O)O ; CN1CCC(c2ccc(Oc3nc(NS(=O)(=O)c4cnn(C)c4)nc(Cl)c3CC(F)(F)F)cc2)CC1 ; CC(C)Cc1ccccc1-c1nc(NS(=O)(=O)c2cnn(C)c2)nc(Oc2ccc(C3CCN(C)CC3)cc2)c1CC(F)(F)F]

A heterogeneous solution of (2-isobutylphenyl)boronic acid (approximately 44.74 mg, 0.2513 mmol), N-[4-chloro-6-[4-(1-methyl-4-piperidyl)phenoxy]-5-(2,2,2-trifluoroethyl)pyrimidin-2-yl]-1-methyl-pyrazole-4-sulfonamide (75 mg, 0.1005 mmol), tetrakis(triphenylphosphine)palladium(0) (approximately 23.23 mg, 0.02010 mmol), and potassium carbonate (approximately 69.45 mg, 0.5025 mmol) in dioxane (418.7 μL) and water (83.77 μL) was microwaved in a sealed vial to 120° C. for 30 minutes. The crude solution was concentrated to ⅓ the volume by blowing a steady stream of air over the surface. The solution was acidified with acetic acid (approximately 120.7 mg, 114.3 μL, 2.010 mmol) and diluted with DMSO (2.0 mL). The sample was purified by reverse phase HPLC (Phenomenex Luna C₁₈ column (75×30 mm, 5 m particle size), gradient: 1-99% acetonitrile in water (5 mM HCl) over 15.0 minutes) to afford the title compound as a white solid. N-[4-(2-isobutylphenyl)-6-[4-(1-methyl-4-piperidyl)phenoxy]-5-(2,2,2-trifluoroethyl)pyrimidin-2-yl]-1-methyl-pyrazole-4-sulfonamide (4.4 mg, 7%). ESI-MS m/z calc. 642.26, found 643.54 (M+1)⁺; Retention time: 1.55 minutes; LC method A.

### Example 307: Preparation of Compound 1083

### Step 1: N-[4-(2-isopropylphenyl)-6-[4-(1-methyl-4-piperidyl)phenoxy]-5-(2,2,2-trifluoroethyl)pyrimidin-2-yl]-1-methyl-pyrazole-4-sulfonamide

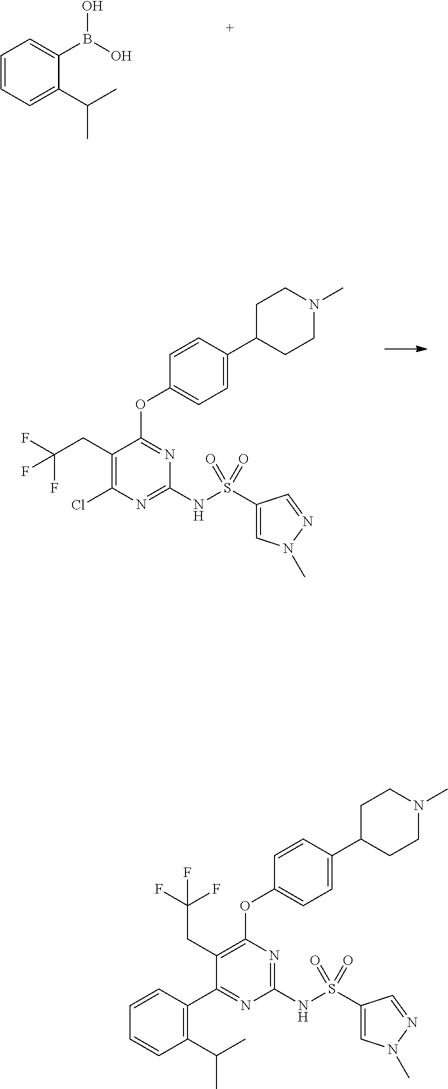 [SMILES: CC(C)c1ccccc1B(O)O ; CN1CCC(c2ccc(Oc3nc(NS(=O)(=O)c4cnn(C)c4)nc(Cl)c3CC(F)(F)F)cc2)CC1 ; CC(C)c1ccccc1-c1nc(NS(=O)(=O)c2cnn(C)c2)nc(Oc2ccc(C3CCN(C)CC3)cc2)c1CC(F)(F)F]

A heterogeneous solution of (2-isopropylphenyl)boronic acid (approximately 41.22 mg, 0.2513 mmol), N-[4-chloro-6-[4-(1-methyl-4-piperidyl)phenoxy]-5-(2,2,2-trifluoroethyl)pyrimidin-2-yl]-1-methyl-pyrazole-4-sulfonamide (75 mg, 0.1005 mmol), tetrakis(triphenylphosphine)palladium(0) (approximately 23.23 mg, 0.02010 mmol), and potassium carbonate (approximately 69.45 mg, 0.5025 mmol) in dioxane (418.7 μL) and water (83.77 μL) was microwaved in a sealed vial to 120° C. for 30 minutes. The crude solution was concentrated to ⅓ the volume by blowing a steady stream of air over the surface. The solution was acidified with acetic acid (approximately 120.7 mg, 114.3 μL, 2.010 mmol) and diluted with DMSO (2.0 mL). The sample was purified by reverse phase HPLC (Phenomenex Luna C₁₈ column (75×30 mm, 5 m particle size), gradient: 1-99% acetonitrile in water (5 mM HCl) over 15.0 minutes) to afford the title compound as a white solid. N-[4-(2-isopropylphenyl)-6-[4-(1-methyl-4-piperidyl)phenoxy]-5-(2,2,2-trifluoroethyl)pyrimidin-2-yl]-1-methyl-pyrazole-4-sulfonamide (6.1 mg, 10%). ESI-MS m/z calc. 628.2443, found 629.4 (M+1)⁺; Retention time: 1.49 minutes; LC method A.

### Example 308: Preparation of Compound 1084

### Step 1: N-[4-chloro-6-[4-(1-methyl-4-piperidyl)phenoxy]-5-(2,2,2-trifluoroethyl)pyrimidin-2-yl]-1-methyl-pyrazole-4-sulfonamide

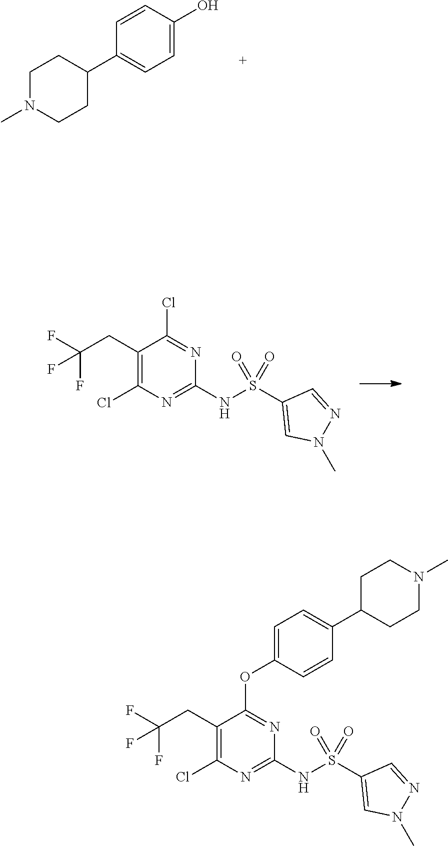 [SMILES: CN1CCC(c2ccc(O)cc2)CC1 ; Cn1cc(S(=O)(=O)Nc2nc(Cl)c(CC(F)(F)F)c(Cl)n2)cn1 ; CN1CCC(c2ccc(Oc3nc(NS(=O)(=O)c4cnn(C)c4)nc(Cl)c3CC(F)(F)F)cc2)CC1]

A heterogeneous solution of N-[4,6-dichloro-5-(2,2,2-trifluoroethyl)pyrimidin-2-yl]-1-methyl-pyrazole-4-sulfonamide (1.5 g, 3.844 mmol), 4-(1-methyl-4-piperidyl)phenol (acetate salt) (966 mg, 3.844 mmol), and potassium carbonate (1.6 g, 11.58 mmol) in NMP (7.6 mL) was heated in a sealed vial to 110° C. for 16 hours. The solution was acidified with acetic acid (3.3 mL, 58.03 mmol) and diluted with water (10 mL). The sample was purified by reverse phase column chromatography (Cis, gradient: 1-99% acetonitrile in water (0.1% formic acid) over 25.0 minutes) to afford N-[4-chloro-6-[4-(1-methyl-4-piperidyl)phenoxy]-5-(2,2,2-trifluoroethyl)pyrimidin-2-yl]-1-methyl-pyrazole-4-sulfonamide (formic acid salt) (1.24 g, 29%) as a white solid. ESI-MS m/z calc. 544.12714, found 545.27 (M+1)⁺; Retention time: 0.49 minutes; LC method D.

### Step 2: N-[4-(2,6-Dimethylphenyl)-6-[4-(1-methyl-4-piperidyl)phenoxy]-5-(2,2,2-trifluoroethyl)pyrimidin-2-yl]-1-methyl-pyrazole-4-sulfonamide

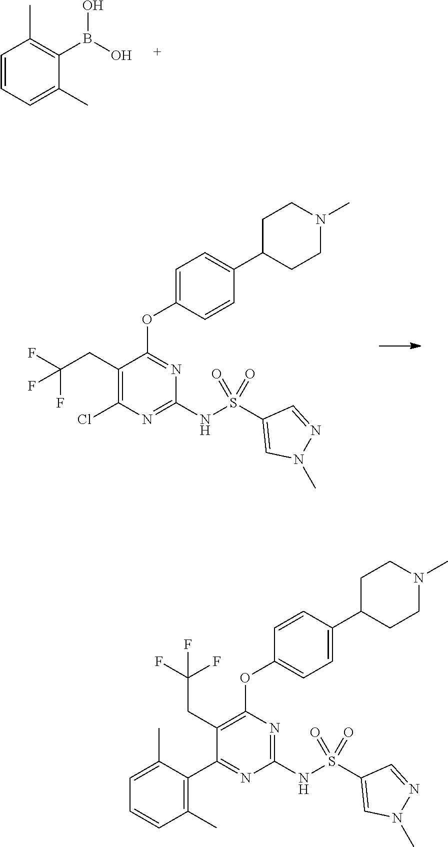 [SMILES: Cc1cccc(C)c1B(O)O ; CN1CCC(c2ccc(Oc3nc(NS(=O)(=O)c4cnn(C)c4)nc(Cl)c3CC(F)(F)F)cc2)CC1 ; Cc1cccc(C)c1-c1nc(NS(=O)(=O)c2cnn(C)c2)nc(Oc2ccc(C3CCN(C)CC3)cc2)c1CC(F)(F)F]

A heterogeneous solution of (2,6-dimethylphenyl)boronic acid (approximately 37.69 mg, 0.2513 mmol), N-[4-chloro-6-[4-(1-methyl-4-piperidyl)phenoxy]-5-(2,2,2-trifluoroethyl)pyrimidin-2-yl]-1-methyl-pyrazole-4-sulfonamide (75 mg, 0.1005 mmol), tetrakis(triphenylphosphine)palladium(0) (approximately 23.23 mg, 0.02010 mmol), and potassium carbonate (approximately 69.45 mg, 0.5025 mmol) in dioxane (418.7 μL) and water (83.77 μL) was microwaved in a sealed vial to 120° C. for 30 minutes. The crude solution was concentrated to ⅓ the volume by blowing a steady stream of air over the surface. The solution was acidified with acetic acid (approximately 120.7 mg, 114.3 μL, 2.010 mmol) and diluted with DMSO (2.0 mL). The sample was purified by reverse phase HPLC (Phenomenex Luna C₁₈ column (75×30 mm, 5 m particle size), gradient: 1-99% acetonitrile in water (5 mM HCl) over 15.0 minutes) to afford the title compound as a white solid. N-[4-(2,6-dimethylphenyl)-6-[4-(1-methyl-4-piperidyl)phenoxy]-5-(2,2,2-trifluoroethyl)pyrimidin-2-yl]-1-methyl-pyrazole-4-sulfonamide (4.2 mg, 7%). ESI-MS m/z calc. 614.2287, found 615.45 (M+1)⁺; Retention time: 1.36 minutes; LC method A.

### Example 309: Preparation of Compound 1085

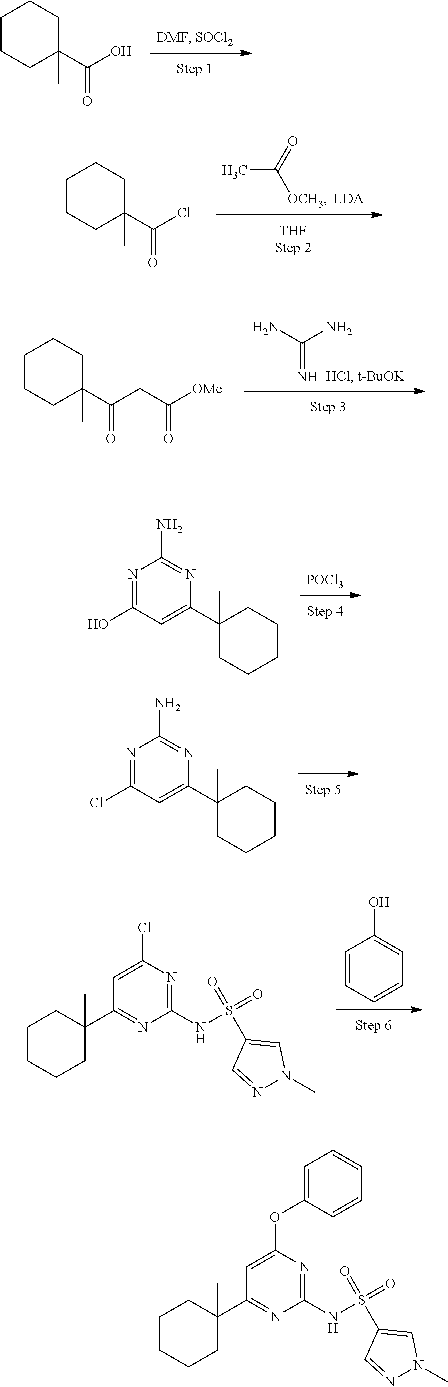

### Step 1: 1-Methylcyclohexanecarbonyl chloride

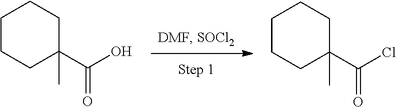

A mixture of 1-methylcyclohexanecarboxylic acid (6.00 g, 42.2 mmol), thionyl chloride (6.02 g, 50.6 mmol) and anhydrous DMF (30.8 mg, 0.42 mmol) was stirred at room temperature overnight. The volatiles were removed under reduced pressure and the crude 1-methylcyclohexanecarbonyl chloride (6.2 g, 91%) was used in the next step without further purification. ¹H NMR (400 MHz, CDCl₃) δ ppm 1.34 (s, 3H), 1.36-1.69 (m, 8H), 2.10-2.21 (m, 2H).

### Step 2: 1-(1-Methylcyclohexyl)pentane-1,3-dione

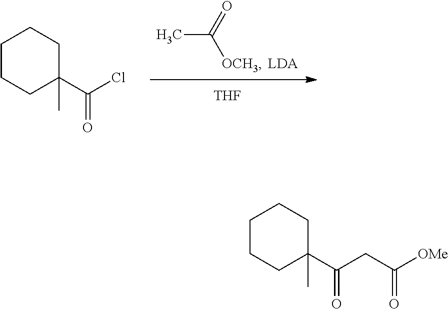

Methyl acetate (2.99 g, 40.36 mmol) was added dropwise at −78° C. to a solution of LDA (40 mL of a 1.0 M solution in THF, 40 mmol), after which a solution of crude 1-methylcyclohexanecarbonyl chloride (3.24 g, 20.18 mmol) in anhydrous THE (20 mL) was also added dropwise at −78° C. and the reaction was allowed to warm to room temperature. The reaction was diluted with 1 N HCl (40 mL) and extracted with ethyl acetate (3×60 mL). The combined organic layers were washed with brine (3×30 mL), dried over anhydrous sodium sulfate, filtered and concentrated under reduced pressure. The crude product (3.40 g, 20.33 mmol) residue was purified by silica gel column chromatography, eluting with 10% EtOAc in heptanes, to give 1-(1-methylcyclohexyl)pentane-1,3-dione (2.52 g, 63% yield) as a yellow oil.

¹H NMR (400 MHz, CDCl₃) δ ppm 1.30 (s, 3H), 1.28-1.47 (m, 6H), 1.50-1.59 (m, 2H), 1.85 -1.95 (m, 2H), 3.54 (s, 2H), 3.73 (s, 3H).

### Step 3: 2-Amino-6-(1-methylcyclohexyl)-pyrimidin-4-ol

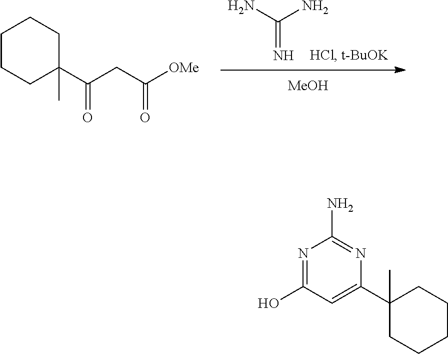

To a solution of 1-(1-methylcyclohexyl)pentane-1,3-dione (5.10 g, 25.7 mmol) and guanidine hydrochloride (2.95 g, 30.9 mmol) in methanol (60 mL) at room temperature was added potassium tert-butoxide (15.59 g, 138.91 mmol) portion-wise over 30 minutes with vigorous stirring, and the reaction was warmed to 60° C. and stirred for 30 minutes. The reaction was then cooled to room temperature and stirred overnight. The precipitated salt was removed by filtration. The solution was concentrated to about 8 mL of methanol, water (8 mL) was added. The mixture was cooled in an ice bath and the pH was adjusted to about 5 by adding 6.0 N HCl. The resulting precipitate was filtered, dried via suction and then under high vacuum. The yellow solid obtained (4.50 g) was triturated in MTBE (15 mL), filtered and dried to afford 2-amino-6-(1-methylcyclohexyl)-pyrimidin-4-ol (4.13 g, 77% yield) as an off-white solid. ¹H NMR (400 MHz, DMSO-d₆) δ ppm 1.05 (s, 3H), 1.26-1.39 (m, 6H), 1.40-1.50 (m, 2H), 1.89-1.99 (m, 2H), 5.48 (s, 1H), 6.34 (s, 2H), 10.55 (s, 1H). [M+H]⁺=208.2.

### Step 4: 2-Amino-4-chloro-6-(1-methylcyclohexyl)pyrimidine

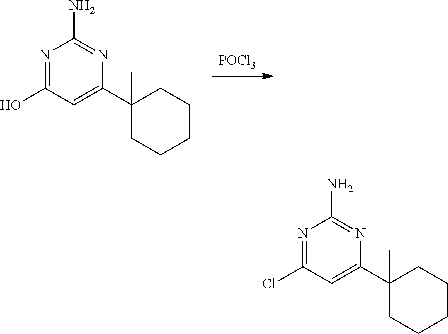

A suspension of 2-amino-6-(1-methylcyclohexyl)-pyrimidin-4-ol (1.50 g, 7.24 mmol) in phosphorus oxychloride (9.99 g, 65.1 mmol) was stirred at 105° C. for 3 hours. The solution was concentrated under vacuum, diluted with dichloromethane (100 mL), cooled to 5° C. and neutralized slowly with 5% KHCO₃ to a pH of 8. The layers were separated, and the aqueous layer was extracted again with dichloromethane (3×50 mL). The combined organic layers were washed with water (2×25 mL), dried over anhydrous sodium sulfate, filtered and concentrated under reduced pressure to give 1.3 g of crude material. The material was purified twice by silica gel column chromatography, eluting with 10% EtOAc in heptanes, to afford 4-chloro-6-(1-methylcyclohexyl)pyrimidin-2-amine (0.81 g, 50% yield) as a white solid. ¹H NMR (300 MHz, CDCl₃) δ ppm 1.15 (s, 3H), 1.27-1.69 (m, 8H), 1.95-2.13 (m, 2H), 5.05 (br. s., 2H), 6.67 (s, 1H). [M+H]⁺+=226.2.

### Step 5: N-[4-chloro-6-(1-methylcyclohexyl)pyrimidin-2-yl]-1-methyl-pyrazole-4-sulfonamide

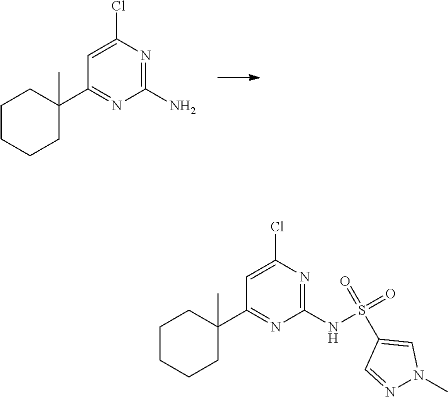 [SMILES: CC1(c2cc(Cl)nc(N)n2)CCCCC1 ; Cn1cc(S(=O)(=O)Nc2nc(Cl)cc(C3(C)CCCCC3)n2)cn1]

To a solution of 4-chloro-6-(1-methylcyclohexyl)pyrimidin-2-amine (0.39 g, 1.728 mmol) in DMF (5.850 mL) at 0° C. was added NaH (270 mg of 60% w/w, 6.751 mmol) and stirred at this temp for 5 minutes. The cooling bath was removed and stirred at room temperature for 20 minutes. The reaction mixture was cooled to 0° C. and 1-methylpyrazole-4-sulfonyl chloride (490 mg, 2.713 mmol) was added in one portion. The reaction mixture was stirred at this temp for 5 minutes, the cooling bath was removed and stirred at room temperature for 30 minutes. The reaction mixture was poured on to ice water, the pH adjusted to ˜ 4 and the precipitate formed was filtered off. The filtrate was washed with water (2×) and dried to give N-[4-chloro-6-(1-methylcyclohexyl)pyrimidin-2-yl]-1-methyl-pyrazole-4-sulfonamide (600 mg, 75%) as a white solid. ESI-MS m/z calc. 369.10263, found 370.2 (M+1)⁺; Retention time: 0.64 minutes; LC method D.

### Step 6: 1-Methyl-N-[4-(1-methylcyclohexyl)-6-phenoxy-pyrimidin-2-yl]pyrazole-4-sulfonamide

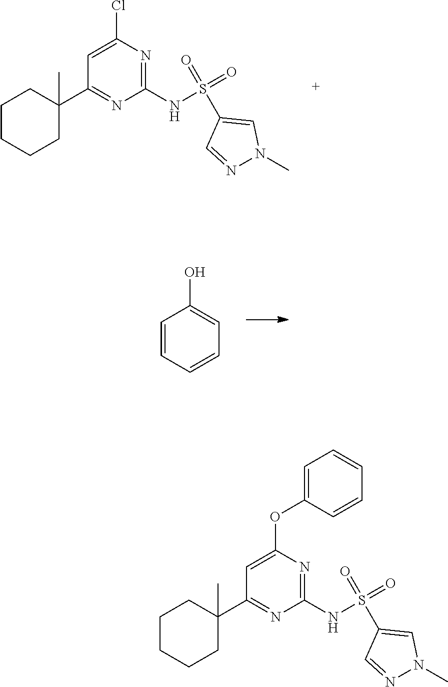 [SMILES: Cn1cc(S(=O)(=O)Nc2nc(Cl)cc(C3(C)CCCCC3)n2)cn1 ; Oc1ccccc1 ; Cn1cc(S(=O)(=O)Nc2nc(Oc3ccccc3)cc(C3(C)CCCCC3)n2)cn1]

To a solution of N-[4-chloro-6-(1-methylcyclohexyl)pyrimidin-2-yl]-1-methyl-pyrazole-4-sulfonamide (20 mg, 0.05407 mmol) in NMP (0.4 mL) was added K₂CO₃ (approximately 37.37 mg, 0.2704 mmol) and phenol (approximately 15.26 mg, 14.40 μL, 0.1622 mmol) and the reaction was stirred at 80° C. for 16 hours. The reaction mixture was diluted with MeOH, filtered and purified by HPLC (1-99% ACN in water (HCl modifier)) to give 1-methyl-N-[4-(1-methylcyclohexyl)-6-phenoxy-pyrimidin-2-yl]pyrazole-4-sulfonamide (11.8 mg, 51%). ESI-MS m/z calc. 427.16782, found 427.68 (M+1)⁺; Retention time: 1.78 minutes; LC method A.

### Example 310: Characterization of Compounds 1086-1092

The compounds in the following tables were prepared in a manner analogous to that described above using commercially available reagents and intermediates described herein.

| | | LCMS | | | |
|---|---|---|---|---|---|
| | | Room | | | |
| Compound | | Temperature | Calc. | | LCMS |
| Number | Structure | (min) | mass | M + 1 | Method |
| 1086 | 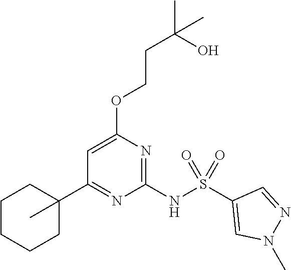 | 1.44 | 437.21 | 438.3 | A |
| 1087 | 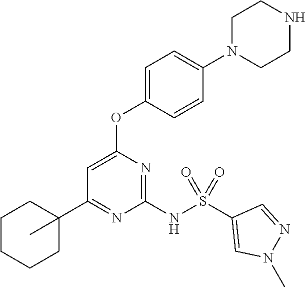 | 1.11 | 511.237 | 511.71 | A |
| 1088 | 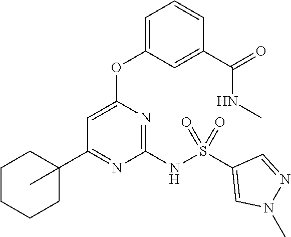 | 1.52 | 484.189 | 485.27 | A |
| 1089 | 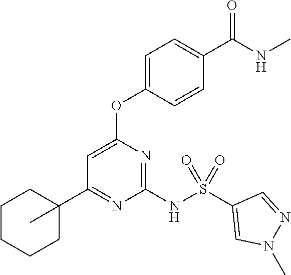 | 1.52 | 484.189 | 484.71 | A |
| 1090 | 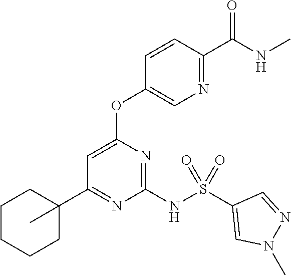 | 1.55 | 485.185 | 486.15 | A |
| 1091 | 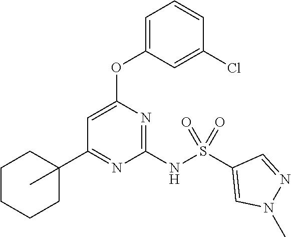 | 1.95 | 461.129 | 462.17 | A |
| 1092 | 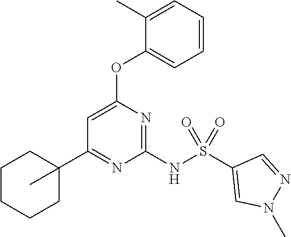 | 1.85 | 441.183 | 442.21 | A |

### Example 311: Preparation of Compound 1093

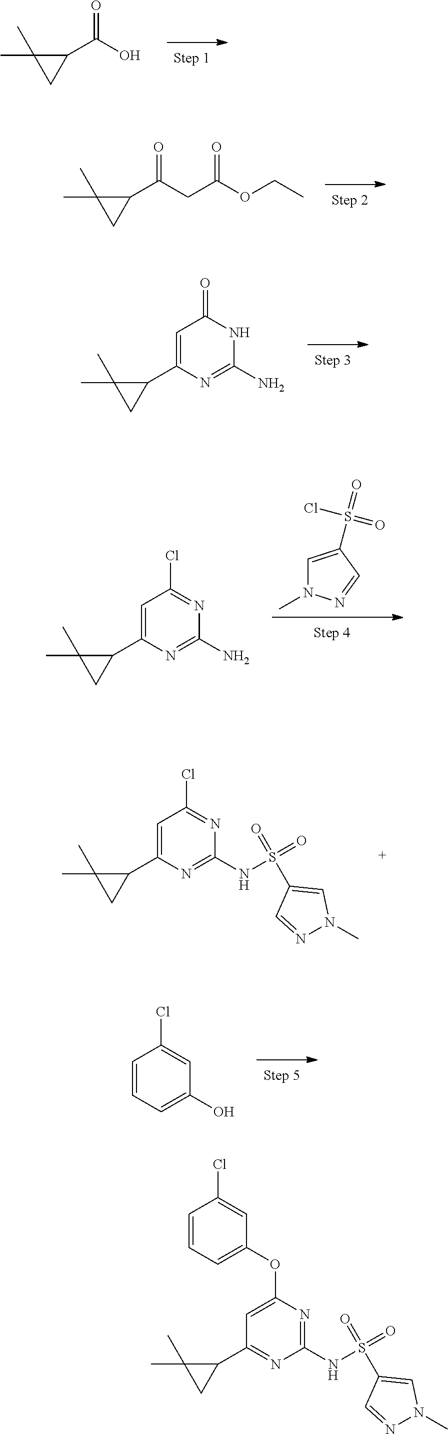

### Step 1: Ethyl 3-(2,2-dimethylcyclopropyl)-3-oxo-propanoate

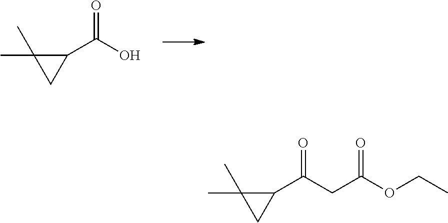

Stage 1: To a solution of 2,2-dimethylcyclopropanecarboxylic acid (4.0 g, 35.04 mmol) and DMF (approximately 128.1 mg, 135.7 μL, 1.752 mmol) in dichloromethane (64.0 mL) at 0° C. was slowly added oxalyl chloride (approximately 26.68 g, 18.34 mL, 210.2 mmol). The reaction was stirred for 1 hour until bubbling ceased. The reaction mixture was concentrated and placed under vacuum for a brief period of time.

Stage 2: To a solution of LDA (approximately 35.39 mL of 2 M, 70.78 mmol) at −78° C. was added dropwise ethyl acetate (approximately 6.267 g, 6.948 mL, 71.13 mmol). After 10 minutes, a solution of the acid chloride from stage 1 dissolved in THE (32 mL) was added dropwise. The reaction was allowed to warm to 23° C. and was quenched with acetic acid (approximately 3.156 g, 2.989 mL, 52.56 mmol). Water was added and the aqueous layer was extracted with ethyl acetate (4×). The combined organics were washed with brine, dried over magnesium sulfate, filtered and concentrated under reduced pressure. The crude residue was purified by flash column chromatography on silica gel (0 to 100% Ethyl acetate in hexanes) to give ethyl 3-(2,2-dimethylcyclopropyl)-3-oxo-propanoate (3.0 g, 42%) as a clear oil. ESI-MS m/z calc. 184.10994, found 185.2 (M+1)⁺; Retention time: 0.5 minutes; LC method D.

### Step 2: 2-Amino-4-(2,2-dimethylcyclopropyl)-1H-pyrimidin-6-one

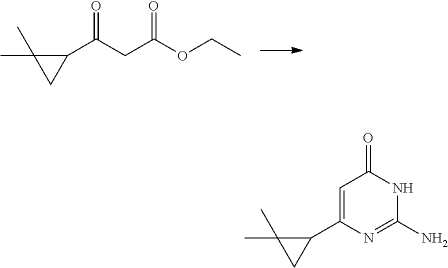

To a solution of ethyl 3-(2,2-dimethylcyclopropyl)-3-oxo-propanoate (3.00 g, 16.28 mmol) and guanidine (hydrochloride salt) (approximately 1.867 g, 19.54 mmol) in methanol (26.44 mL) at 23° C. was added potassium tert-butoxide (approximately 9.865 g, 87.91 mmol) portionwise. The reaction was heated to 85° C. for 12 hours in a pressure vessel. The reaction was cooled to 0° C. and 7 mL of acetic acid was added and then further diluted with 50 mL of methanol. The crude mixture was concentrated on to silica gel. The separation was performed by flash column chromatography on silica gel (10% methanol in dichloromethane). 2-amino-4-(2,2-dimethylcyclopropyl)-1H-pyrimidin-6-one (1.24 g, 22%) was isolated as a white solid. ESI-MS m/z calc. 179.10587, found 180.26 (M+1)⁺; Retention time: 0.25 minutes; LC method D.

### Step 3: 4-Chloro-6-(2,2-dimethylcyclopropyl)pyrimidin-2-amine

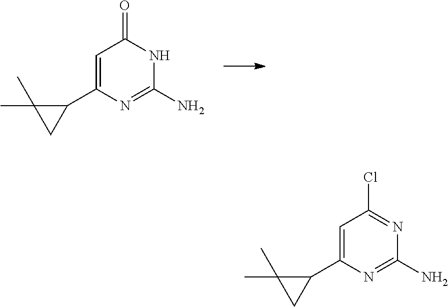

A heterogeneous solution of 2-amino-4-(2,2-dimethylcyclopropyl)-1H-pyrimidin-6-one (1.24 g, 6.919 mmol) in POCl₃ (approximately 12.73 g, 7.739 mL, 83.03 mmol) was heated to 95° C. for 2 hour. The excess POCl₃ was removed in vacuo. The crude residue was dissolved in dichloromethane and a saturated aqueous solution of sodium bicarbonate was added. The biphasic mixture was stirred rapidly for 30 minutes, at which point the organic layer was removed and the aqueous layer was further extracted with dichloromethane (4×). The combined organics were dried over sodium sulfate, filtered, and concentrated in vacuo. The crude residue was separated by flash column chromatography on silica gel (20% ethyl acetate in hexanes) to give 4-chloro-6-(2,2-dimethylcyclopropyl)pyrimidin-2-amine (340 mg, 20%) as a yellow oil ESI-MS m/z calc. 197.07198, found 198.16 (M+1)⁺; Retention time: 1.11 minutes; LC method A. ¹H NMR (400 MHz, Chloroform-d) δ 6.54 (s, 1H), 5.15 (s, 2H), 1.74 (dd, J=8.0, 5.7 Hz, 1H), 1.30 (dd, J=5.7, 4.5 Hz, 1H), 1.23 (s, 3H), 1.03 (s, 3H), 0.89 (dd, J=8.0, 4.4 Hz, 1H).

### Step 4: N-[4-Chloro-6-(2,2-dimethylcyclopropyl)pyrimidin-2-yl]-1-methyl-pyrazole-4-sulfonamide

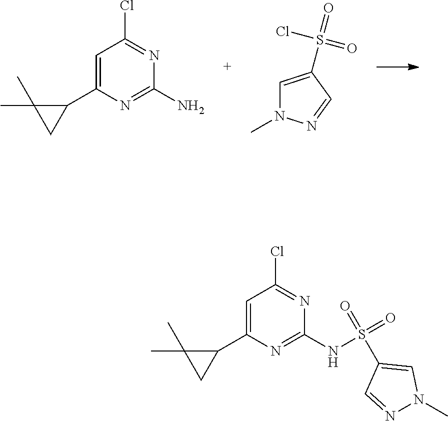

To a solution of 4-chloro-6-(2,2-dimethylcyclopropyl)pyrimidin-2-amine (340 mg, 1.720 mmol) in DMF (6.880 mL) at 0° C. was added sodium hydride (approximately 165.1 mg, 6.880 mmol) and the reaction mixture was stirred at this temperature for 5 minutes, then removed from the cooling bath and stirred at room temperature for 10 minutes. The reaction mixture was cooled to 0° C. and a solution of 1-methylpyrazole-4-sulfonyl chloride (approximately 621.3 mg, 3.440 mmol) in DMF (3.0 mL) was added dropwise over 1 minutes. The reaction mixture was stirred at this temperature for 5 minutes, then removed from the cooling bath and stirred at room temperature for 12 minutes. The reaction mixture was cooled back to 0° C. and quenched with hydrochloric acid (approximately 762.7 μL of 37% w/v, 7.740 mmol), then diluted with a solution of ethyl acetate/hexanes (1:1) and partitioned with a saturated aqueous solution of sodium bicarbonate. The organic layer was removed, and the aqueous layer was further extracted with ethyl acetate/hexanes (1:1, 5×). The combined organic extracts were dried with brine and magnesium sulfate. The solution was filtered, and the filtrate was concentrated in vacuo. The crude residue was purified by flash column chromatography on silica gel (5 to 40% ethyl acetate in hexanes). N-[4-chloro-6-(2,2-dimethylcyclopropyl)pyrimidin-2-yl]-1-methyl-pyrazole-4-sulfonamide (350 mg, 42%) was isolated as a white solid. ESI-MS m/z calc. 341.07132, found 342.3 (M+1)⁺; Retention time: 0.55 minutes; LC method D.

### Step 5: N-[4-(3-Chlorophenoxy)-6-(2,2-dimethylcyclopropyl)pyrimidin-2-yl]-1 -methyl-pyrazole-4-sulfonamide

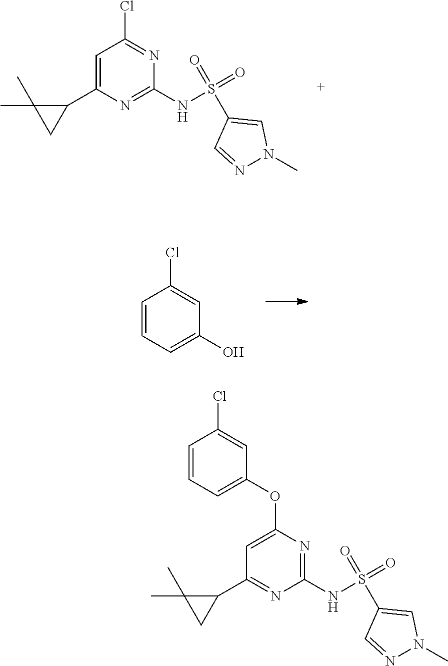

A heterogenous solution of N-[4-chloro-6-(2,2-dimethylcyclopropyl)pyrimidin-2-yl]-1-methyl-pyrazole-4-sulfonamide (20.0 mg, 0.05851 mmol), 3-chlorophenol (approximately 22.56 mg, 0.1755 mmol), and cesium carbonate in NMP (400 μL) was sealed in a vial and the reaction was heated at 110° C. for 16 hours. The reaction was cooled and diluted with DMS0, filtered, and purified by reverse phase chromatography (acetonitrile in water with 0.1% hydrochloric acid). N-[4-(3-chlorophenoxy)-6-(2,2-dimethylcyclopropyl)pyrimidin-2-yl]-1 -methyl-pyrazole-4-sulfonamide (14.3 mg, 56% ). ESI-MS m/z calc. 433.09753, found 434.36 (M+1)⁺; Retention time: 1.62 minutes; LC method A.

### Example 312: Characterization of Compounds 1094-1103

The compounds in the following tables were prepared in a manner analogous to that described above using commercially available reagents and intermediates described herein.

| | | LCMS | | | | |
|---|---|---|---|---|---|---|
| | | Room | | | | |
| Compound | | Temp. | Calc. | | LCMS | |
| Number. | Structure | (min) | mass | M + 1 | Method | NMR |
| 1094 | 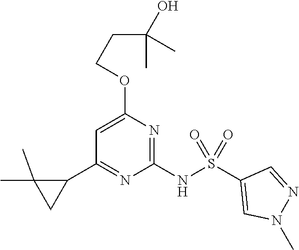 | 1.11 | 409.178 | 410.41 | A | |
| 1095 | 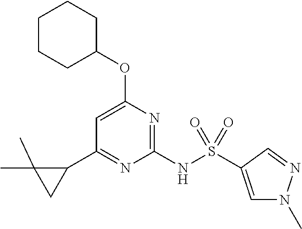 | 1.55 | 405.183 | 406.45 | A | |
| 1096 | 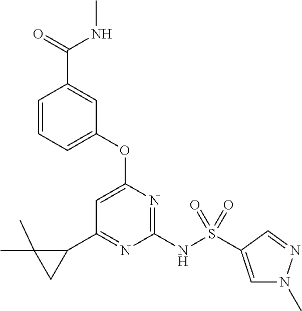 | 1.18 | 456.158 | 457.47 | A | |
| 1097 | 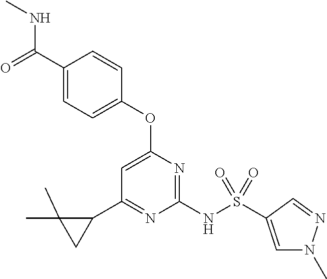 [SMILES: CNC(=O)c1ccc(Oc2cc(C3CC3(C)C)nc(NS(=O)(=O)c3cnn(C)c3)n2)cc1] | 1.16 | 456.158 | 457.43 | A | |
| 1098 | 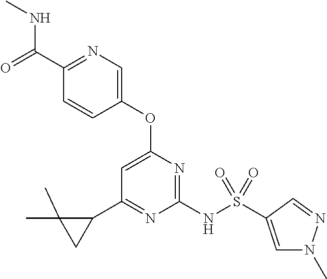 [SMILES: CNC(=O)c1ccc(Oc2cc(C3CC3(C)C)nc(NS(=O)(=O)c3cnn(C)c3)n2)cn1] | 1.17 | 457.153 | 458.45 | A | |
| 1099 | 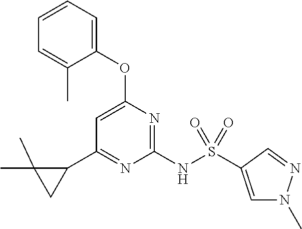 | 1.52 | 413.152 | 414.4 | A | |
| 1100 | 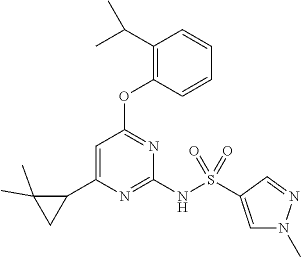 | 1.74 | 441.183 | 442.45 | A | |
| 1101 | 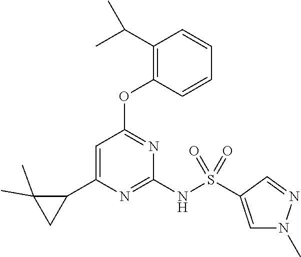 | 1.43 | 399.137 | 400.39 | A | |
| 1102 | 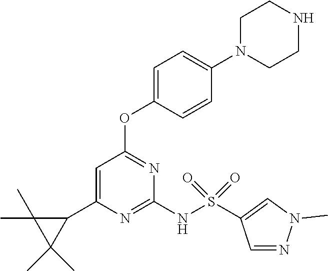 | 1.32 | 511.237 | 512 | A | ¹H NMR (400 MHz, DMSO-d₆) δ 13.63 (s, 1H), 8.86 (s, 1H), 8.39 (s, 1H), 7.95 (s, 1H), 7.83 (s, 1H), 7.13 (d, J = 9.0 Hz, 2H), 7.01 (d, J = 9.1 Hz, 2H), 6.36 (s, 1H), 3.92 (s, 3H), 3.25 (s, 4H), 2.98 (s, 4H), 1.60 (s, 1H), 1.23 (s, 6H), 1.08 (s, 6H). |
| 1103 | 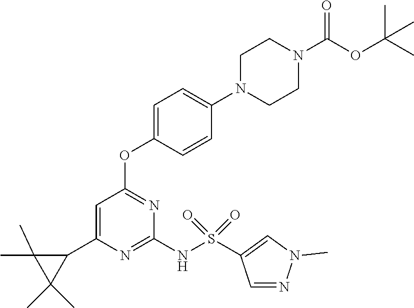 [SMILES: Cn1cc(S(=O)(=O)Nc2nc(Oc3ccc(N4CCN(C(=O)OC(C)(C)C)CC4)cc3)cc(C3C(C)(C)C3(C)C)n2)cn1] | 1.92 | 611.289 | 612 | A | ¹H NMR (400 MHz, DMSO-d₆) δ 11.37 (s, 1H), 7.77 (s, 1H), 7.42 (s, 1H), 7.08 (t, J = 11.2 Hz, 4H), 6.30 (s, 1H), 3.75 (s, 3H), 3.48 (s, 4H), 3.12 (s, 4H), 1.43 (s, 9H), 1.18 (s, 6H), 1.09 (s, 6H). |

### Example 313: Preparation of Compound 1104

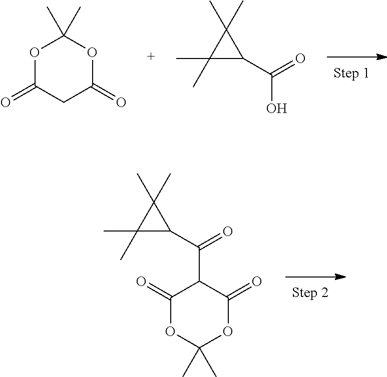

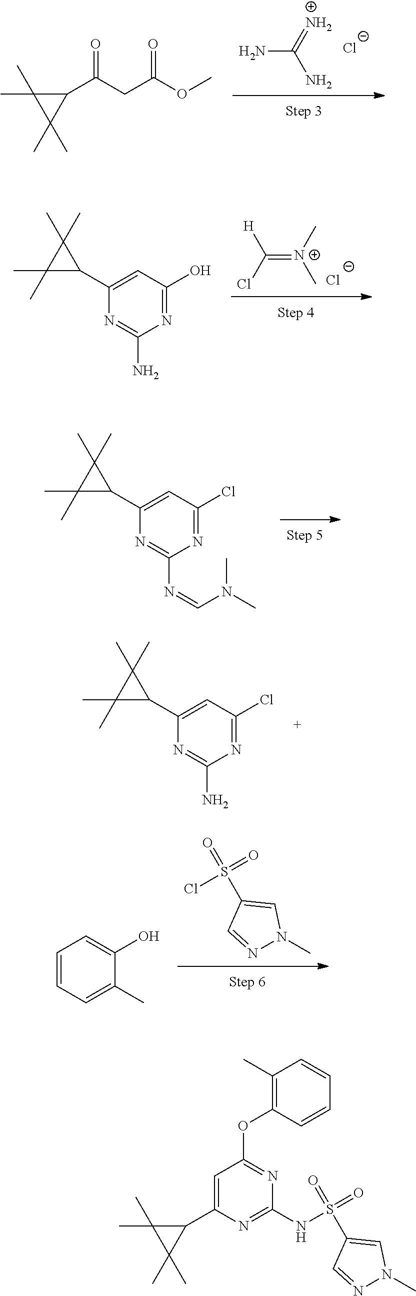

### Step 1: 2,2-Dimethyl-5-(2,2,3,3-tetramethylcyclopropanecarbonyl)-1,3-dioxane-4,6-dione

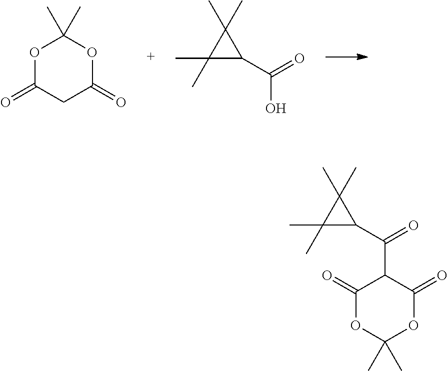

To a solution of 2,2,3,3 tetramethylcyclopropanecarboxylic acid (9.99 g, 70.26 mmol) and 2,2-dimethyl-1,3-dioxane-4,6-dione (11.08 g, 76.88 mmol) in dichloromethane (125 mL) was added 4-dimethylaminopyridin (12.85 g, 105.2 mmol). After cooling the mixture to 0° C., N-(3-Dimethylaminopropyl)-N′-ethylcarbodiimide hydrochloride (19.00 g, 99.11 mmol) was added in portions and then the reaction mixture was stirred at room temperature for 0.5 hour then heated at 30° C. for 3 hours. The reaction was then stirred at room temperature overnight. The mixture was treated with distilled water (125 mL) and HCl 1 N (75 mL) and then the layers were separated. The organic layer was washed with HCl 1 M (50 mL), with distilled water (100 mL) then dried over anhydrous sodium sulfate, filtered and concentrated under reduced pressure to provide the desired product 2,2-dimethyl-5-(2,2,3,3-tetramethylcyclopropanecarbonyl)-1,3-dioxane-4,6-dione (18.96 g, 90% yield, 90% pure) as a red oil. ¹H NMR (300 MHz, CDCl₃) δ ppm 1.51 (s, 6H), 1.52 (s, 6H), 1.90 (s, 6H), 1.96 (s, 1H), 3.13 (d, J=1.5 Hz, 1H). ESI-MS m/z found 267.1 (M−1); Retention time: 2.34 minutes. LCMS Method: Kinetex C₁₈ 4.6 ×50 mm 2.6 μM, 2.0 mL/min, 95% H₂O (0.1% FA)⁺5% CH₃CN (0.1% FA) to 95% CH₃CN (0.1% FA) gradient (2.0 min) then hold at 95% CH₃CN (0.1% FA) for 1.0 minute.

### Step 2: Methyl 3-oxo-3-(2,2,3,3-tetramethylcyclopropyl)propanoate

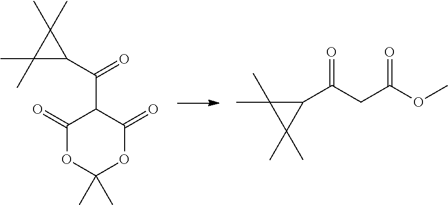

2,2-Dimethyl-5-(2,2,3,3-tetramethylcyclopropanecarbonyl)-1,3-dioxane-4,6-dione (18.96 g, 70.67 mmol) was dissolved in anhydrous methanol (200 mL) and then stirred overnight for 4 hours at 50° C. The reaction mixture was concentrated under reduced pressure to provide the desired product methyl 3-oxo-3-(2,2,3,3 tetramethylcyclopropyl)propanoate (14 g, 86%) as an orange oil. ¹H NMR (300 MHz, CDCl₃) δ ppm 1.20 (s, 6H), 1.20 (s, 6H), 1.53 (s, 1H), 3.49 (s, 2H), 3.66-3.80 (m, 3H). ESI-MS m/z found 199.2 (M+1)⁺; Retention time: 2.04 minutes. LC method C.

### Step 3: 2-Amino-6-(2,2,3,3-tetramethylcyclopropyl)pyrimidin-4-ol

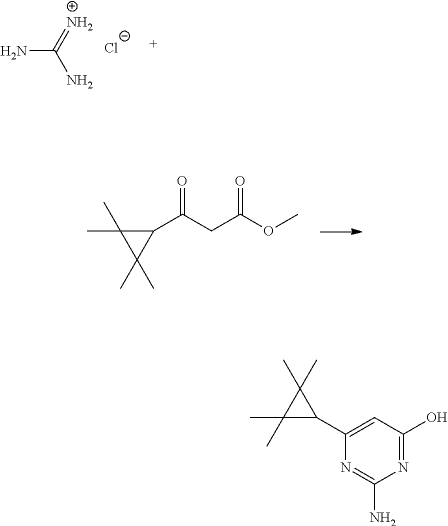

Under nitrogen atmosphere, heptane washed, sodium metal (2.30 g, 100 mmol) was dissolved in absolute ethanol (110 mL) and guanidine hydrochloride (9.29 g, 97.3 mmol) was added and the resulting suspension was stirred 5 minutes and then a solution of methyl 3-oxo-3-(2,2,3,3-tetramethylcyclopropyl)propanoate (12.48 g, 63.0 mmol) in ethanol (10 mL) was added. The reaction was then left stirring at 80° C. for 2 hours then at room temperature overnight. The reaction mixture was concentrated then dissolved in distilled water (50 mL) and the resulting solution was acidified with 3 N HCl and the resulting precipitate, filtered, washed with diethylether. The resulting powder was dried under reduced pressure to provide the desired product 2-amino-6-(2,2,3,3-tetramethylcyclopropyl)pyrimidin-4-ol (8.41 g, 64%) as a pale yellow powder. ¹H NMR (300 MHz, DMSO-d₆) ppm 1.08 (s, 6H), 1.13 (s, 6H), 1.20 (s, 1H), 5.37 (d, J=0.9 Hz, 1H), 6.69 (br. s., 2H), 7.00 (br. s., 1H). ESI-MS m/z found 208.3 (M+1)⁺; Retention time: 1.23 minute. LC method C.

### Step 4: N′-[4-chloro-6-(2,2,3,3-tetramethylcyclopropyl)pyrimidin-2-yl]-N,N-dimethyl-formamidine

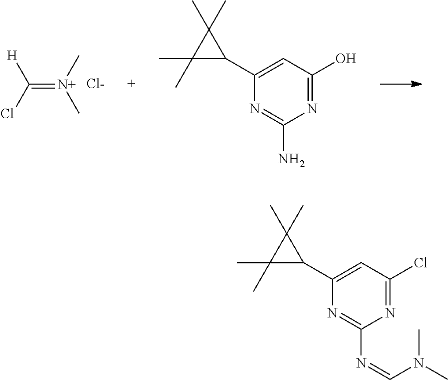

2-Amino-6-(2,2,3,3-tetramethylcyclopropyl)pyrimidin-4-ol (4.0 g, 19 mmol ) was added under nitrogen to (chloromethylene)dimethyliminium chloride (11.6 g, 90.7 mmol) in chloroform (125 ml). The reaction mixture was subsequently heated at reflux for 4 hours, during which time the starting material completely dissolved. The reaction mixture was cooled to the room temperature and a thick precipitate formed. The reaction mixture was cooled further in a cold water bath and then the solid that formed was filtered and washed with methanol (3×25 mL) then dried under high vacuum to provide the desired product N′-[4-chloro-6-(2,2,3,3-tetramethylcyclopropyl)pyrimidin-2-yl]-N,N-dimethyl-formamidine (5.12 g, 95%) as a white solid, (hydrated form). ESI-MS m/z calc. 280.14548, found 281.2 (M+1)⁺; Retention time: 1.53 minutes; LC method C. ¹H NMR (300 MHz, DMSO-d₆) ppm 1.23 (s, 6H), 1.24 (s, 6H), 1.76 (s, 1H), 3.31 (s, 3H), 3.43 (s, 3H), 7.45 (s, 1H), 8.31 (s, 1H), 8.82 (s, 2H), 11.80-12.59 (m, 1H).

### Step 5: 4-Chloro-6-(2,2,3,3-tetramethylcyclopropyl)pyrimidin-2-amine

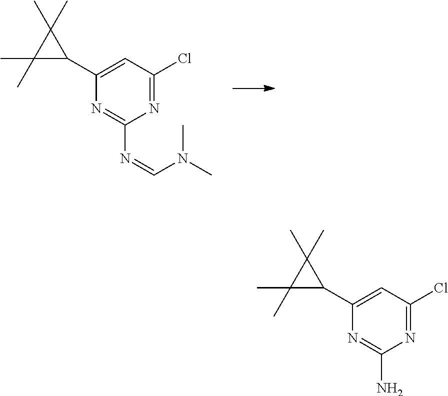

N′-[4-chloro-6-(2,2,3,3-tetramethylcyclopropyl)pyrimidin-2-yl]-N,N-dimethyl-formamidine (4.5 g, 16.0 mmol) in ethanol (40 mL) was added concentrated HCl (6.0 mL, 58 mmol) and then stirred at 50° C. for 4 hours. The reaction mixture was concentrated under reduced pressure, the resulting solid was triturated in methanol, a small amount of water was added and the solid formed was filtered and washed with distilled water then dried under vacuum. To provide more sample, distilled water was added to the filtrate and the solid obtained (batch 2) was dried to air. The filtrate was neutralized with concentrated aqueous sodium bicarbonate resulted in more material precipitating. All the material and previous crops were combined and partitioned between ethyl acetate (200 mL) and aqueous saturated sodium bicarbonate (50 mL). The organic phase was separated, dried over anhydrous sodium sulfate, filtered and concentrated under reduced pressure and the resulting residue was purified on silica gel using 15 to 25% ethyl acetate in heptane to provide the desired product 4-chloro-6-(2,2,3,3-tetramethylcyclopropyl)pyrimidin-2-amine (1.04 g, 28%) as a white solid. ¹H NMR (300 MHz, CDCl₃) ppm 1.20 (s, 6H), 1.24 (s, 6H), 1.35 (s, 1H), 5.03 (br. s., 2H), 6.54 (s, 1H). ESI-MS m/z found 226.2 (M+1)⁺; Retention time: 3.06 minutes, LC method H.

### Step 6: 1-Methyl-N-[4-(2-methylphenoxy)-6-(2,2,3,3-tetramethylcyclopropyl)pyrimidin-2-yl]pyrazole-4-sulfonamide

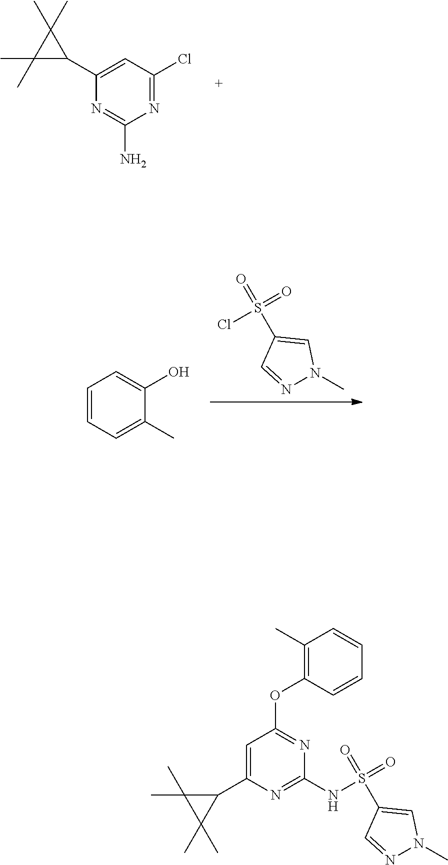

Stage 1: NaH (35 mg of 60% w/w, 0.8751 mmol) was added to o-cresol (50 mg, 0.4624 mmol) in NMP (500 μL) at 0° C. The mixture was stirred for 15 minutes. then added to 4-chloro-6-(2,2,3,3-tetramethylcyclopropyl)pyrimidin-2-amine (50 mg, 0.2215 mmol) in NMP (500 μL). The resulting mixture was stirred at 110° C. overnight. The reaction mixture was cooled down, filtered, and purified on reverse phase HPLC (HCl modifier, 15-75% ACN-H₂O) to give 4-(2-methylphenoxy)-6-(2,2,3,3-tetramethylcyclopropyl)pyrimidin-2-amine (35.4 mg, 52%) ESI-MS m/z calc. 297.1841, found 298.0 (M+1)⁺; Retention time: 1.37 minutes (LC method A).

Stage 2: NaH (36 mg of 60% w/w, 0.9001 mmol) was added to 4-(2-methylphenoxy)-6-(2,2,3,3-tetramethylcyclopropyl)pyrimidin-2-amine (35.4 mg, 52%) in DMF (1 mL). The mixture was stirred at room temperature for 30 minutes. then 1-methylpyrazole-4-sulfonyl chloride (80 mg, 0.4429 mmol) in DMF (1 mL) was added. The mixture was stirred at room temperature. The reaction mixture was stirred at 70° C. for 30 minutes, filtered and purified on reverse phase HPLC (HCl modifier, 15-75% ACN-H₂O) to give 1-methyl-N-[4-(2-methylphenoxy)-6-(2,2,3,3-tetramethylcyclopropyl)pyrimidin-2-yl]pyrazole-4-sulfonamide (2.4 mg, 2%). ESI-MS m/z calc. 441.18347, found 442.0 (M+1)⁺; Retention time: 1.74 minutes; LC method A. ¹H NMR (400 MHz, DMSO-d₆) δ 11.29 (s, 1H), 7.51 (s, 1H), 7.34 (d, J=7.5 Hz, 1H), 7.30-7.25 (m, 1H), 7.21 (s, 1H), 7.09 (d, J=9.0 Hz, 1H), 6.46 (d, J=20.2 Hz, 2H), 3.68 (s, 3H), 2.04 (s, 3H), 1.46 (s, 1H), 1.12 (s, 6H), 1.02 (d, J=26.2 Hz, 6H).

### Example 314: Preparation of Compound 1105

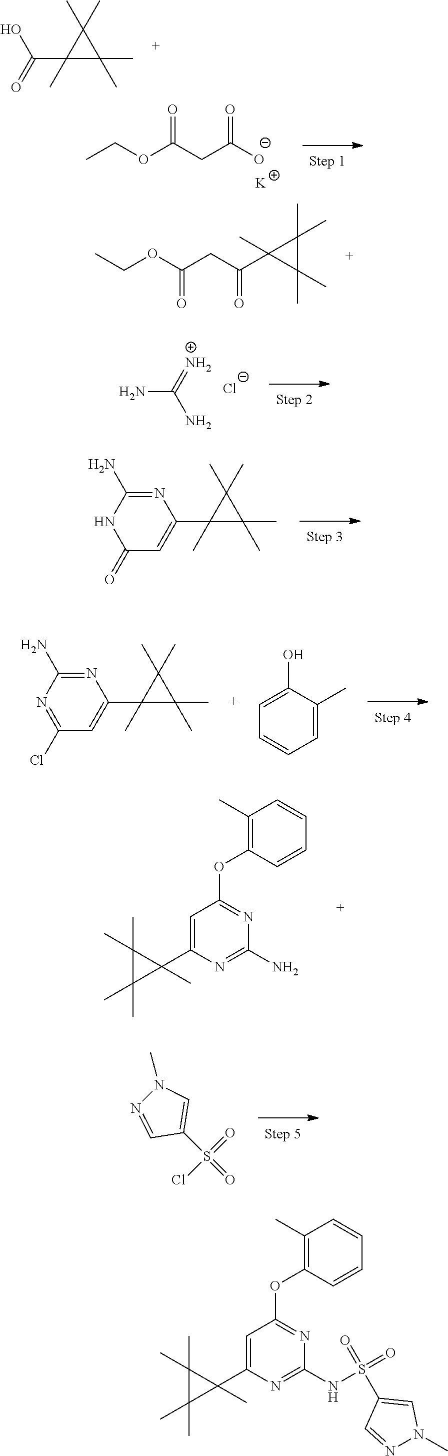

### Step 1: Ethyl 3-oxo-3-(1,2,2,3,3-pentamethylcyclopropyl)propanoate

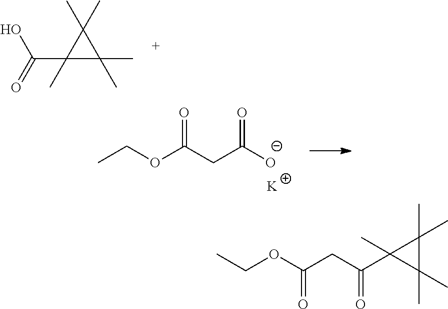

A solution of 1,2,2,3,3-pentamethylcyclopropanecarboxylic acid (3.12 g, 20.0 mmol) in THE (50 mL) was treated with carbonyl diimidazole (3.43 g, 21.2 mmol) and left to stir at room temperature for 2 hours. Magnesium chloride was added (2.09 g, 22.0 mmol) followed by ethyl potassium malonate (3.74 g, 22.0 mmol) and the reaction was heated in an oil bath at 50° C. overnight. Once cooled to room temperature, the reaction mixture was transferred to a 500-mL separatory funnel with methyl tert-butyl ether (300 mL) and washed with 1 N HCl (2×100 mL). The organic layer was then washed with water (100 mL), brine (100 mL), dried over sodium sulfate, filtered and concentrated under reduced pressure. The residue was purified by silica gel chromatography on a 40-g column (Biotage SP1), eluting from 0% to 20% ethyl acetate in heptanes, to afford ethyl 3-oxo-3-(1,2,2,3,3-pentamethylcyclopropyl)propanoate (480 mg, 11% yield) as a pale orange oil. ESI-MS m/z calc. 226.15689, found 227.2 (M+1)⁺; Retention time: 2.21 minutes; LC method C.

### Step 2: 2-Amino-4-(1,2,2,3,3-pentamethylcyclopropyl)-1H-pyrimidin-6-one

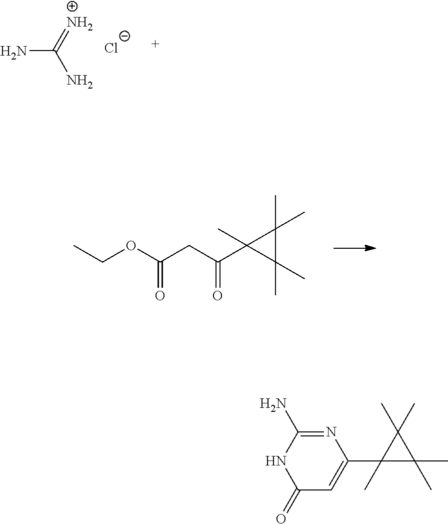

A flame-dried 50-mL flask was charged with anhydrous ethanol (6.0 mL) and sodium metal (95 mg, 4.1 mmol, pre-washed with heptanes) was added. Once gas evolution had stopped and all the sodium had reacted, guanidine hydrochloride (374 mg, 3.92 mmol) was added. After 5 minutes, a solution of ethyl 3-oxo-3-(1,2,2,3,3-pentamethylcyclopropyl)propanoate (571 mg, 2.52 mmol) in anhydrous ethanol (1.5 mL, +1.0 mL rinse) was added and the reaction was heated in an oil bath at 80° C. for 21 hours. Once cooled, the solvent was removed under reduced pressure, suspended in water (about 15 mL) and acidified to a pH of 2-3 with concentrated HCl. The mixture was transferred to a separatory funnel and extracted with a mixture of isopropanol in chloroform (1:2, 3×20 mL). The combined organic layers were dried over sodium sulfate, filtered and concentrated under reduced pressure to afford a slightly gummy solid. This solid was triturated with methyl tert-butyl ether (about 15 mL), washed with additional methyl tert-butyl ether and dried under high vacuum to afford 2-amino-4-(1,2,2,3,3-pentamethylcyclopropyl)-1H-pyrimidin-6-one (142 mg, 25% yield) as a white solid. ESI-MS m/z calc. 221.15282, found 222.2 (M+1)⁺; Retention time: 1.28 minutes; LC method C. ¹H NMR (300 MHz, DMSO-d₆) δ ppm 0.99 (s, 6H), 1.04 (s, 6H), 1.19 (s, 3H), 5.42 (s, 1H), 7.15 (br.s, 2H).

### Step 3: 4-Chloro-6-(1,2,2,3,3-pentamethylcyclopropyl)pyrimidin-2-amine

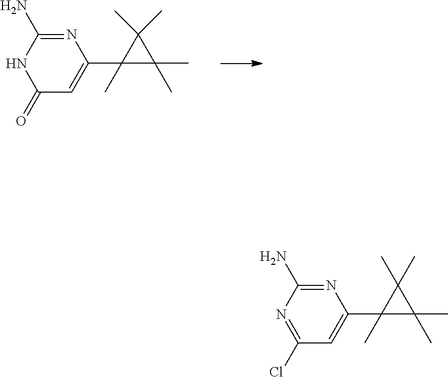 [SMILES: CC1(C)C(C)(C)C1(C)c1cc(=O)[nH]c(N)n1 ; CC1(C)C(C)(C)C1(C)c1cc(Cl)nc(N)n1]

A suspension of 2-amino-4-(1,2,2,3,3-pentamethylcyclopropyl)-1H-pyrimidin-6-one (142 mg, 0.642 mmol) in dioxane (2.4 mL) and phosphorus oxychloride (0.60 mL, 6.4 mmol) was gradually heated up to 80° C. in an oil bath. After heating for 3.5 hours the reaction was removed from the oil bath and left to cool to room temperature. The reaction was quenched by adding portionwise to a 5% aqueous sodium bicarbonate (50 mL) cooled in an ice bath. The aqueous layer was then transferred to a 125-mL separatory funnel and extracted with dichloromethane (3×20 mL). The combined organic layers were dried over sodium sulfate, filtered and concentrated under reduced pressure. The residue was purified by silica gel chromatography on a 24-g column (Biotage SP1), eluting from 0% to 30% ethyl acetate in heptanes, to afford 4-chloro-6-(1,2,2,3,3-pentamethylcyclopropyl)pyrimidin-2-amine (96 mg, 62% yield) as a white solid. ESI-MS m/z calc. 239.11893, found 240.2 (M+1)⁺; Retention time: 2.97 minutes; LC method H. ¹H NMR (300 MHz, CDCl₃) δ ppm 0.97 (s, 6H), 1.12 (s, 6H), 1.24 (s, 3H), 5.31 (br. s., 2H), 6.44 (s, 1H).

### Step 4: 4-(2-Methylphenoxy)-6-(1,2,2,3,3-pentamethylcyclopropyl)pyrimidin-2-amine

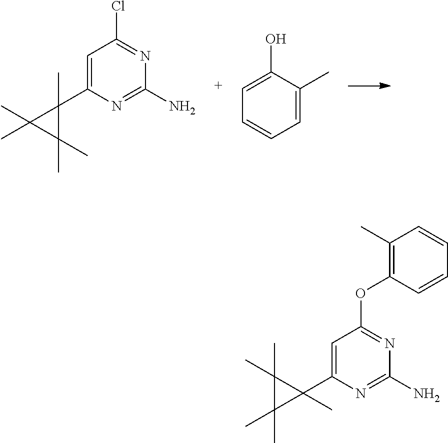

NaH (41.8 mg of 60% w/w, 1.05 mmol) was added to o-cresol (78 mg, 0.72 mmol) in NMP (500 μL) at 0° C. The mixture was stirred for 45 minutes. then added to 4-chloro-6-(1,2,2,3,3-pentamethylcyclopropyl)pyrimidin-2-amine (77 mg, 0.3212 mmol) in NMP (500 μL). The resulting mixture was stirred at 100° C. for 20 hours. The reaction mixture was cooled down, filtered, and purified on reverse phase HPLC (HCl modifier, 10-60% ACN-H₂O) to give 4-(2-methylphenoxy)-6-(1,2,2,3,3-pentamethylcyclopropyl)pyrimidin-2-amine (45.2 mg, 43%). ESI-MS m/z calc. 311.19977, found 312.0 (M+1)⁺; Retention time: 1.39 minutes; LC method A.

### Step 5: 1-Methyl-N-[4-(2-methylphenoxy)-6-(1,2,2,3,3-pentamethylcyclopropyl)pyrimidin-2-yl]pyrazole-4-sulfonamide

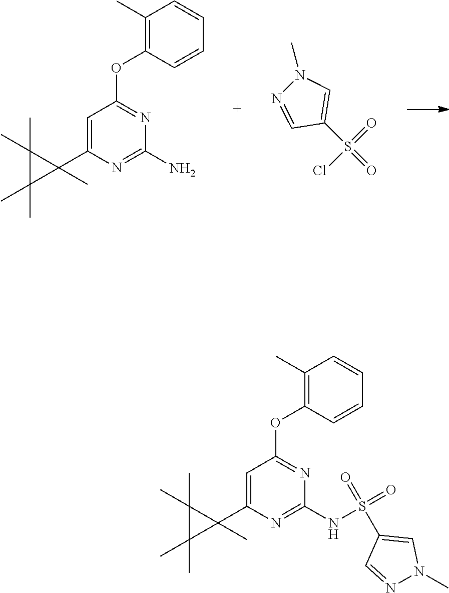

To a solution of 4-(2-methylphenoxy)-6-(1,2,2,3,3-pentamethylcyclopropyl)pyrimidin-2-amine (22.6 mg, 0.06967 mmol) in DMF (500 μL) at 0° C. was added NaH (13.4 mg of 60% w/w, 0.335 mmol), and the reaction mixture was stirred at this temperature for 5 minutes. The reaction mixture was removed from the cooling bath and stirred at room temperature for 30 minutes. 1-methylpyrazole-4-sulfonyl chloride (17 mg, 0.094 mmol) in DMF (500 μL) was added slowly to the previous mixture and the resulting mixture was stirred at 100° C. 50 minutes. The crude was filtered and purified by purified on reverse phase HPLC (HCl modifier, 25-75% ACN-H₂O) to give 1-methyl-N-[4-(2-methylphenoxy)-6-(1,2,2,3,3-pentamethylcyclopropyl)pyrimidin-2-yl]pyrazole-4-sulfonamide (5.5 mg, 17%). ESI-MS m/z calc. 455.1991, found 456.0 (M+1)⁺; Retention time: 1.74 minutes; LC method A. ¹H NMR (400 MHz, Methanol-d₄) δ 7.44-7.40 (m, 1H), 7.37 (td, J=7.5, 1.8 Hz, 1H), 7.32 (td, J=7.4, 1.5 Hz, 1H), 7.20-7.10 (m, 3H), 6.42 (d, J=0.8 Hz, 1H), 3.76 (s, 3H), 2.17 (s, 3H), 1.32 (s, 3H), 1.17 (s, 6H), 1.04 (s, 6H).

### Example 315: Preparation of Compound 1106

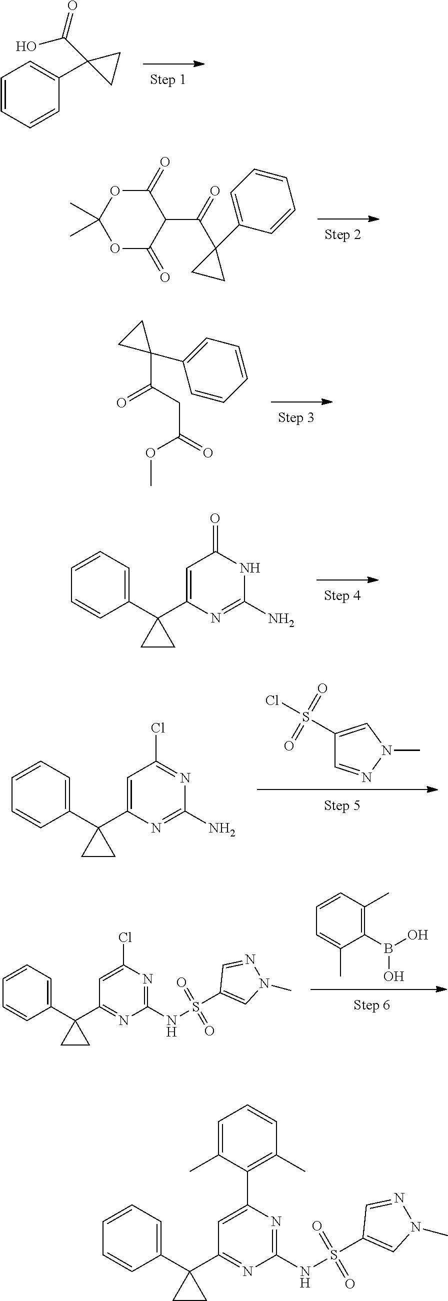

### Step 1: 2,2-Dimethyl-5-(1-phenylcyclopropanecarbonyl)-1,3-dioxane-4,6-dione

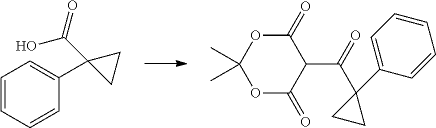 [SMILES: O=C(O)C1(c2ccccc2)CC1 ; CC1(C)OC(=O)C(C(=O)C2(c3ccccc3)CC2)C(=O)O1]

A solution of 1-phenylcyclopropanecarboxylic acid (7.16 g, 44.2 mmol), 2,2-dimethyl-1,3-dioxane-4,6-dione (7.00 g, 48.6 mmol) and 4-(dimethylamino)pyridine (8.09 g, 66.2 mmol) in dichloromethane (75 mL) was cooled in an ice bath and treated with N-(3-dimethylaminopropyl)-N′-ethylcarbodiimide hydrochloride (11.9 g, 62.1 mmol). After 5 minutes, the ice bath was removed, and the reaction was stirred at room temperature for about 68 hours. The crude was transferred to a 1.0 L separatory funnel with water (400 mL) and dichloromethane (about 200 mL). After extraction, the layers were separated and the organic layer was washed with 1 N HCl (2×100 mL), brine (100 mL), dried over sodium sulfate, filtered and concentrated under reduced pressure to afford crude 2,2-dimethyl-5-(1-phenylcyclopropanecarbonyl)-1,3-dioxane-4,6-dione (11.06 g, 85% purity, 74% yield) as an orange solid. ESI-MS m/z calc. 288.09976, found 287.1 (M−1)⁺; Retention time: 2.17 minutes; LC method C. ¹H NMR (300 MHz, CDCl₃) δ ppm 1.46-1.50 (m, 2H), 1.52-1.56 (m, 2H), 1.60 (s, 6H), 7.18-7.31 (m, 3H), 7.37-7.42 (m, 2H).

### Step 2: Methyl 3-oxo-3-(1-phenylcyclopropyl)propanoate

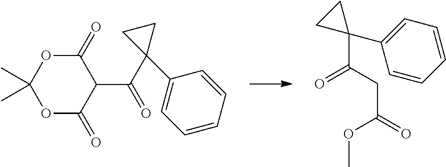 [SMILES: CC1(C)OC(=O)C(C(=O)C2(c3ccccc3)CC2)C(=O)O1 ; COC(=O)CC(=O)C1(c2ccccc2)CC1]

A solution of crude 2,2-dimethyl-5-(1-phenylcyclopropanecarbonyl)-1,3-dioxane-4,6-dione (11.06 g, 38.36 mmol) in methanol (120 mL) was refluxed for 21 hours. The reaction mixture was concentrated under reduced pressure and kept under high vacuum to afford crude methyl 3-oxo-3-(1-phenylcyclopropyl)propanoate (8.51 g, 93% purity, 95% yield) as an orange oil that was used in the following step without further purification. ESI-MS m/z calc. 218.0943, found 219.2 (M+1)⁺; Retention time: 1.95 minutes; LC method C. ¹H NMR (300 MHz, CDCl₃) δ ppm 1.23-1.29 (m, 2H), 1.67-1.73 (m, 2H), 3.35 (s, 2H), 3.64 (s, 3H), 7.26-7.40 (m, 5 H).

### Step 3: 2-Amino-4-(1-phenylcyclopropyl)-1H-pyrimidin-6-one

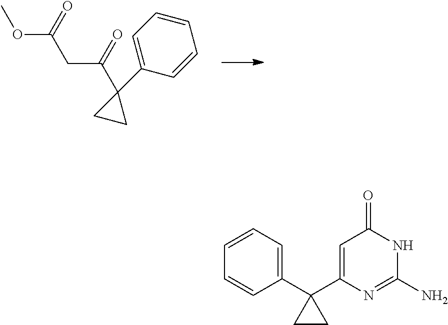 [SMILES: COC(=O)CC(=O)C1(c2ccccc2)CC1 ; Nc1nc(C2(c3ccccc3)CC2)cc(=O)[nH]1]

A flame-dried 500-mL flask was charged with anhydrous ethanol (75 mL) and sodium metal (1.46 g, 63.5 mmol, pre-washed with heptanes) was added. Once gas evolution had stopped and all the sodium had reacted, guanidine hydrochloride (5.78 g, 60.5 mmol) was added. After 5 minutes, a solution of crude methyl 3-oxo-3-(1-phenylcyclopropyl)propanoate (8.51 g, 39.0 mmol) in anhydrous ethanol (5 mL, +5 mL rinse) was added and the reaction was heated in an oil bath at 80° C. for 18 hours. Once cooled, the solvent was removed under reduced pressure, suspended in water (100 mL) and acidified to a pH of 1-2 with concentrated HCl. The solids were filtered, washed with water and dried under high vacuum to afford 2-amino-4-(1-phenylcyclopropyl)-1H-pyrimidin-6-one (6.41 g, 72% yield) as a pale yellow solid. ESI-MS m z calc. 227.10587, found 228.2 (M+1)⁺; Retention time: 1.36 minutes; LC method C. ¹H NMR (300 MHz, DMSO-d₆) δ ppm 1.04-1.11 (m, 2H), 1.47-1.55 (m, 2H), 4.76 (s, 1H), 6.40 (br.s, 2H), 7.24-7.40 (m, 5H), 10.5 (br.s, 1H).

### Step 4: 4-chloro-6-(1-phenylcyclopropyl)pyrimidin-2-amine

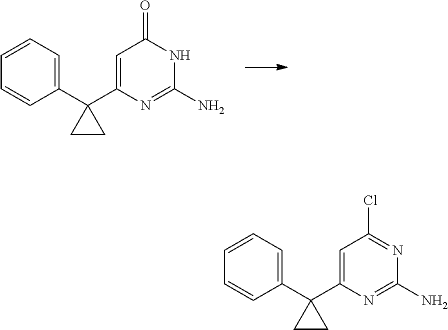 [SMILES: Nc1nc(C2(c3ccccc3)CC2)cc(=O)[nH]1 ; Nc1nc(Cl)cc(C2(c3ccccc3)CC2)n1]

A suspension of 2-amino-4-(1-phenylcyclopropyl)-1H-pyrimidin-6-one (3.50 g, 15.4 mmol) in dioxane (56 mL) and phosphorus oxychloride (14 mL) was gradually heated up to 80° C. in an oil bath. After heating for 3-3.5 hours the reaction was removed from the oil bath and left to stir at room temperature overnight (about 17 hours). The reaction was quenched by adding portionwise to 5% aqueous sodium bicarbonate (700 mL) cooled in an ice bath (solid sodium hydroxide was added in order to maintain the pH at about 7-8). The aqueous layer was then transferred to a 1.0 L separatory funnel and extracted with dichloromethane (3×150 mL). The combined organic layers were dried over sodium sulfate, filtered and concentrated under reduced pressure. The residue was purified by silica gel chromatography on an 80 g column (Biotage SP1), eluting from 0% to 40% ethyl acetate in heptanes, to afford 4-chloro-6-(1-phenylcyclopropyl)pyrimidin-2-amine (2.39 g, 98.3% purity, 62% yield) as a pale brown solid. ESI-MS m/z calc. 245.07198, found 246.1 (M+1)⁺; Retention time: 3.11 minutes; LC method H.

¹H NMR (300 MHz, DMSO-d₆) δ ppm 1.21-1.30 (m, 2H), 1.54-1.63 (m, 2H), 5.79 (s, 1H), 7.01 (br. s, 2H), 7.28-7.45 (m, 5H).

### Step 5: N-[4-chloro-6-(1-phenylcyclopropyl)pyrimidin-2-yl]-1-methyl-pyrazole-4-sulfonamide

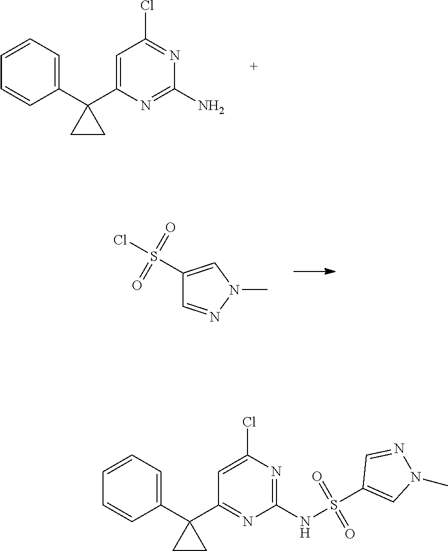

To a solution of 4-chloro-6-(1-phenylcyclopropyl)pyrimidin-2-amine (490 mg, 1.675 mmol) in DMF (6.7 mL) at 0° C. was added sodium hydride (268 mg of 60% w/w, 6.701 mmol). The reaction was stirred for 15 minutes before adding 1-methylpyrazole-4-sulfonyl chloride (605 mg, 3.350 mmol) in one portion. The reaction was further stirred for 15 minutes and then quenched with the addition of acetic acid (800 μL, 14.07 mmol). The solution was partitioned between ethyl acetate and water. The water layer was removed, and the organic layer was washed with brine (2×), dried over magnesium sulfate, filtered, and concentrated in vacuo. The crude residue was separated by flash column chromatography on silica gel (gradient: 10 to 65% ethyl acetate in hexanes) to afford N-[4-chloro-6-(1-phenylcyclopropyl)pyrimidin-2-yl]-1 -methyl-pyrazole-4-sulfonamide (310 mg, 47%) as a white solid. ESI-MS m/z calc. 389.07132, found 390.11 (M+1)⁺; Retention time: 0.67 minutes; LC method D.

### Step 6: N-[4-(2,6-Dimethylphenyl)-6-(1-phenylcyclopropyl)pyrimidin-2-yl]-1-methyl-pyrazole-4-sulfonamide

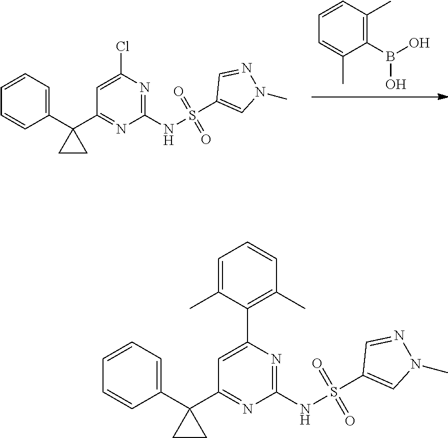 [SMILES: Cn1cc(S(=O)(=O)Nc2nc(Cl)cc(C3(c4ccccc4)CC3)n2)cn1 ; Cc1cccc(C)c1B(O)O ; Cc1cccc(C)c1-c1cc(C2(c3ccccc3)CC2)nc(NS(=O)(=O)c2cnn(C)c2)n1]

A heterogeneous solution of N-[4-chloro-6-(1-phenylcyclopropyl)pyrimidin-2-yl]-1 -methyl-pyrazole-4-sulfonamide (38.2 mg, 0.09798 mmol), (2,6-dimethylphenyl)boronic acid (29.4 mg, 0.1960 mmol), tetrakis(triphenylphosphine)palladium(0) (22.7 mg, 0.01964 mmol), and potassium carbonate (54.2 mg, 0.3922 mmol) in dioxane (400 μL) and water (80 μL) was microwaved in a sealed vial to 125° C. for 25 minutes. The reaction was acidified with acetic acid (110 μL, 1.934 mmol). The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-(2,6-dimethylphenyl)-6-(1-phenylcyclopropyl)pyrimidin-2-yl]-1-methyl-pyrazole-4-sulfonamide (4.6 mg, 10%) as a white solid. ESI-MS m/z calc. 459.17288, found 460.0 (M+1)⁺; Retention time: 1.95 minutes (LC method A). ¹H NMR (400 MHz, Chloroform-d) δ 7.86 (d, J=1.0 Hz, 2H), 7.33 (d, J=3.9 Hz, 4H), 7.30-7.23 (m, 1H), 7.16 (dd, J=8.1, 7.0 Hz, 1H), 7.07-6.99 (m, 2H), 6.30 (s, 1H), 3.90 (s, 3H), 1.97 (s, 6H), 1.74 (q, J=3.6 Hz, 2H), 1.43 (q, J=3.7 Hz, 2H).

### Example 316: Preparation of Compound 1107

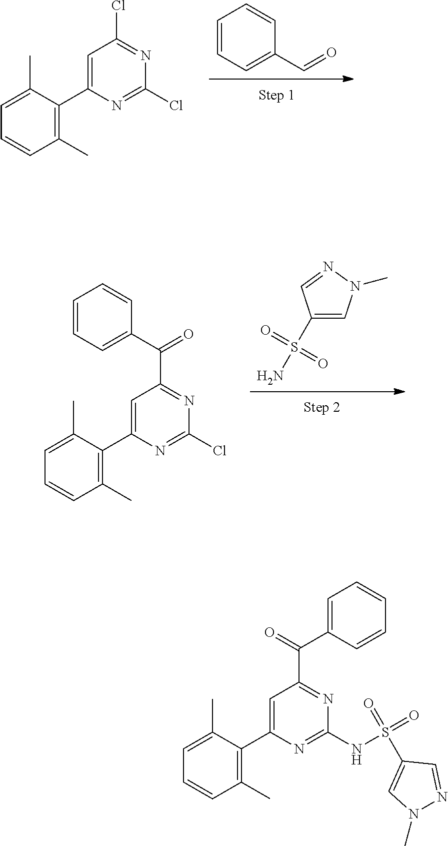

### Step 1: [2-Chloro-6-(2,6-dimethylphenyl)pyrimidin-4-yl]-phenyl-methanone

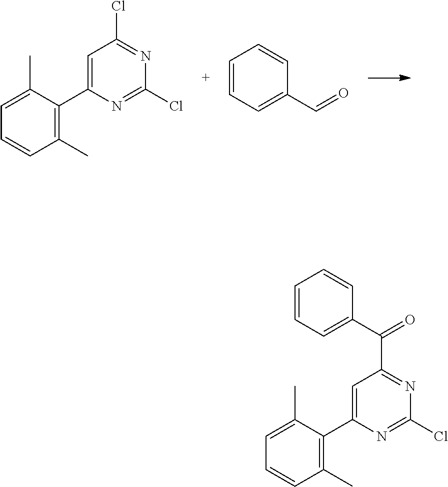 [SMILES: Cc1cccc(C)c1-c1cc(Cl)nc(Cl)n1 ; O=Cc1ccccc1 ; Cc1cccc(C)c1-c1cc(C(=O)c2ccccc2)nc(Cl)n1]

To a solution of 2,4-dichloro-6-(2,6-dimethylphenyl)pyrimidine (141 mg, 0.5570 mmol), benzaldehyde (65 mg, 0.6125 mmol), and 1,3-dimethylbenzimidazol-3-ium iodide (84 mg, 0.3065 mmol) in DMF (9.3 mL) was added sodium hydride (40 mg of 60% w/w, 1.000 mmol). The reaction was stirred for 16 hours and quenched with acetic acid (130 μL, 2.286 mmol). The solution was partitioned between ethyl acetate and water. The water layer was removed, and the organic layer was washed with brine (2×), dried over magnesium sulfate, filtered and concentrated in vacuo. The crude residue was separated by flash column chromatography on silica gel (5 to 15% ethyl acetate in hexanes) to afford [2-chloro-6-(2,6-dimethylphenyl)pyrimidin-4-yl]-phenyl-methanone (44 mg, 21%) as a yellow solid. ESI-MS m z calc. 322.08728, found 323.14 (M+1)⁺; Retention time: 0.81 minutes; LC method D.

### Step 2: N-[4-Benzoyl-6-(2,6-dimethylphenyl)pyrimidin-2-yl]-1-methyl-pyrazole-4-sulfonamide

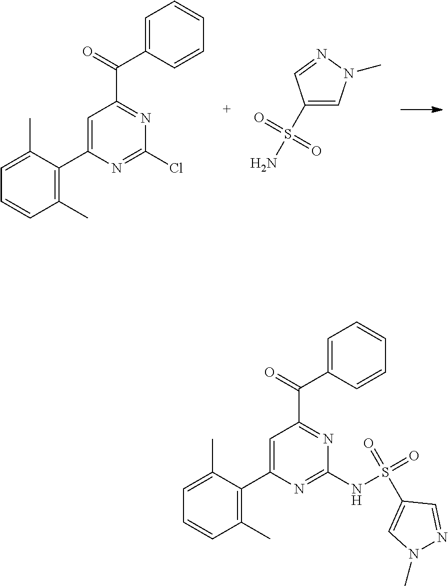

A heterogeneous solution of [2-chloro-6-(2,6-dimethylphenyl)pyrimidin-4-yl]-phenyl-methanone (6 mg, 0.01859 mmol), 1-methylpyrazole-4-sulfonamide (9.0 mg, 0.05584 mmol), diacetoxypalladium (0.8 mg, 0.003563 mmol), (5-diphenylphosphanyl-9,9-dimethyl-xanthen-4-yl)-diphenyl-phosphane (4.3 mg, 0.007431 mmol) and potassium carbonate (7.7 mg, 0.05571 mmol) in dioxane (310 μL) was microwaved to 125° C. for 25 minutes. The solvent was removed under a steady stream of air. The crude mixture was dissolved in acetic acid (50.0 μL, 0.8792 mmol) and DMSO (1.0 mL) and filtered. The sample was purified by reverse phase HPLC (Waters Sunfire C₁₈ column (100×50 mm, 10 m particle size), gradient: 1-99% acetonitrile in water (5 mM HCl) over 15.0 minutes) to afford N-[4-benzoyl-6-(2,6-dimethylphenyl)pyrimidin-2-yl]-1-methyl-pyrazole-4-sulfonamide (1.8 mg, 22%) as a yellow solid. ESI-MS m/z calc. 447.1365, found 448.27 (M+1)⁺; Retention time: 1.76 minutes; LC method A. ¹H NMR (400 MHz, Chloroform-d) δ 8.20 (dd, J=8.3, 1.4 Hz, 2H), 8.07 (s, 1H), 7.78-7.69 (m, 1H), 7.67 (s, 1H), 7.61 (dd, J=8.4, 7.1 Hz, 2H), 7.51 (d, J=7.6 Hz, 2H), 7.26 (dt, J=8.0, 5.1 Hz, 1H), 7.14 (d, J=7.6 Hz, 2H), 3.70 (s, 3H), 2.10 (s, 6H).

### Example 317: Preparation of Compound 1108

### Step 1: N-(4-Chloro-5,6-dimethyl-pyrimidin-2-yl)benzenesulfonamide

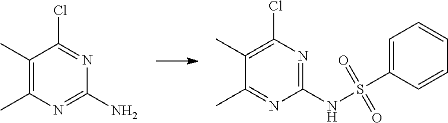

To a solution of 4-chloro-5,6-dimethyl-pyrimidin-2-amine (400 mg, 2.538 mmol) in DMA (3.5 mL) was added NaH (102 mg of 60% w/w, 2.550 mmol). The reaction was stirred at room temperature for 15 minutes. benzenesulfonyl chloride (325 μL, 2.547 mmol) was added and the reaction was stirred at room temperature for 15 hours. The reaction was quenched with MeOH and the solvent was evaporated under reduced pressure. EtOAc was added to the reaction and washed with water (×3). The organic layer was dried over Na₂SO₄, filtered and concentrated. The crude product was purified on 80 g of silica gel utilizing a gradient of 0-50% ethyl acetate in hexane to yield N-(4-chloro-5,6-dimethyl-pyrimidin-2-yl)benzenesulfonamide (114 mg, 15%) as a white solid. ESI-MS m/z calc. 297.03387, found 298.1 (M+1)⁺; Retention time: 1.33 minutes; LC method A.

### Step 2: N-(4,5-Dimethyl-6-phenyl-pyrimidin-2-yl)benzenesulfonamide

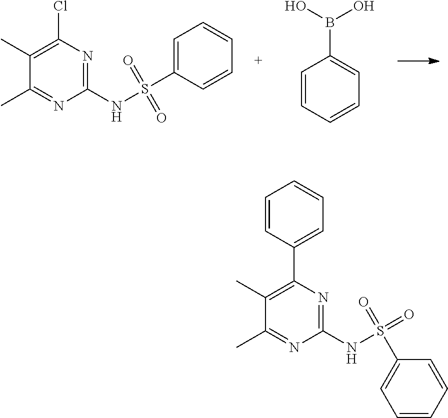 [SMILES: Cc1nc(NS(=O)(=O)c2ccccc2)nc(Cl)c1C ; OB(O)c1ccccc1 ; Cc1nc(NS(=O)(=O)c2ccccc2)nc(-c2ccccc2)c1C]

The mixture of N-(4-chloro-5,6-dimethyl-pyrimidin-2-yl)benzenesulfonamide (50 mg, 0.1679 mmol), phenylboronic acid (34 mg, 0.2788 mmol), Pd(dppf)C₁₂ (20 mg, 0.02733 mmol), dioxane (1.5 mL) and potassium carbonate (approximately 169.0 μL of 2 M, 0.3380 mmol) was degassed by flow of nitrogen and stirred at 110° C. for 1 hour in a pressure vessel. The cooled mixture was filtered and concentrated in vacuo. The crude product was dissolved in DMSO, filtered and purified using a reverse phase HPLC C₁₈ column and a dual gradient run from 1-99% mobile phase B over 15 minutes (Mobile phase A=H₂O (5 mM HCl). Mobile phase B═CH₃CN) to yield N-(4,5-dimethyl-6-phenyl-pyrimidin-2-yl)benzenesulfonamide (37.2 mg, 63%) as a cream solid. ¹H NMR (400 MHz, DMSO-d₆) δ 11.54 (s, 1H), 8.03-7.88 (m, 2H), 7.68-7.60 (m, 1H), 7.60-7.53 (m, 2H), 7.53-7.44 (m, 3H), 7.41-7.30 (m, 2H), 2.38 (s, 3H), 2.10 (s, 3H). ESI-MS m/z calc. 339.10416, found 340.2 (M+1)⁺; Retention time: 1.51 minutes; LC method A.

### Example 318: Preparation of Compound 1109, Compound 1110, and Compound 146

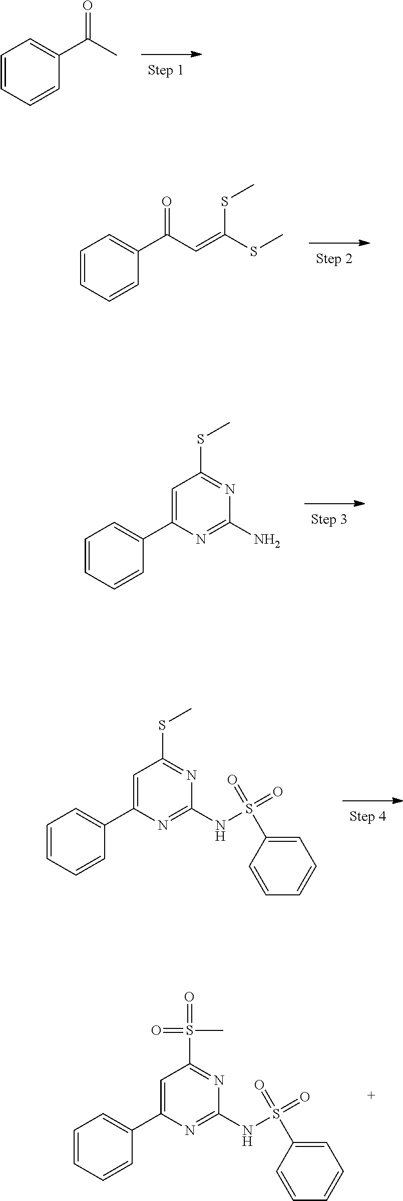

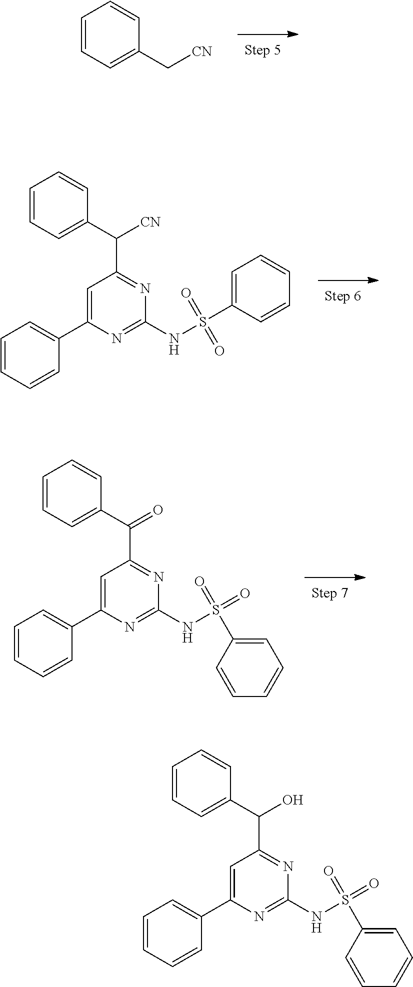 [SMILES: N#CCc1ccccc1 ; N#CC(c1ccccc1)c1cc(-c2ccccc2)nc(NS(=O)(=O)c2ccccc2)n1 ; O=C(c1ccccc1)c1cc(-c2ccccc2)nc(NS(=O)(=O)c2ccccc2)n1 ; O=S(=O)(Nc1nc(-c2ccccc2)cc(C(O)c2ccccc2)n1)c1ccccc1]

### Step 1: 3,3-Bis(methylsulfanyl)-1-phenyl-prop-2-en-1-one

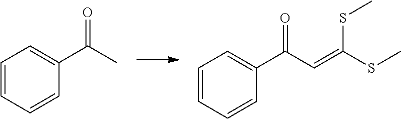

To a solution of 1-phenylethanone (10 g, 83.23 mmol) in THE (100 mL) was added sodium hydride (approximately 6.659 g of 60% w/w, 166.5 mmol) at 0° C. The mixture was stirred at room temperature for 30 minutes. It was cooled back to 0° C. and added carbon disulfide (approximately 6.971 g, 5.506 mL, 91.55 mmol) and was stirred for 30 minutes at room temperature. Then it was cooled back to 0° C. and iodomethane was added (approximately 29.54 g, 12.96 mL, 208.1 mmol). The reaction mixture was stirred at room temperature for 3 hours. The reaction mixture was slowly poured into ice cold water and was extracted with ethyl acetate. The organic layer was separated, dried over Na₂SO₄, concentrated and the residue was dissolved in minimum amount of DCM and hexane was added. The resulting solid was filtered to afford 3,3-bis(methylsulfanyl)-1-phenyl-prop-2-en-1-one (12.2 g, 65%). ESI-MS m/z calc. 224.03296, found 225.09 (M+1)⁺; Retention time: 0.58 minutes; LC method D.

### Step 2: 4-Methylsulfanyl-6-phenyl-pyrimidin-2-amine

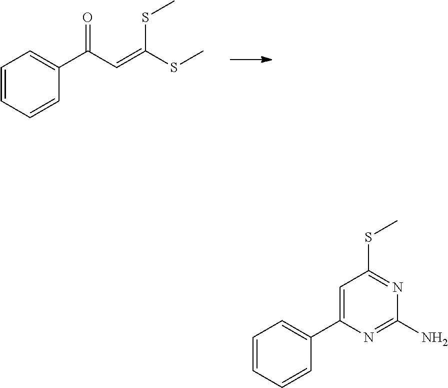

To a solution of 3,3-bis(methylsulfanyl)-1-phenyl-prop-2-en-1-one (4 g, 17.83 mmol) in dimethylformamide (40 mL) was added 3,3-bis(methylsulfanyl)-1-phenyl-prop-2-en-1-one (4 g, 17.83 mmol) and potassium carbonate (approximately 9.857 g, 71.32 mmol). The mixture was heated at 100° C. overnight. The reaction mixture was cooled and was added water. Scratched the sides with a spatula. Product got crashed out which was filtered, and the precipitate was collected to afford 4-methylsulfanyl-6-phenyl-pyrimidin-2-amine (1.8 g, 46%). ESI-MS m z calc. 217.06737, found 218.12 (M+1)⁺; Retention time: 0.36 minutes; LC method D.

### Step 3: N-(4-methylsulfanyl-6-phenyl-pyrimidin-2-yl)benzenesulfonamide

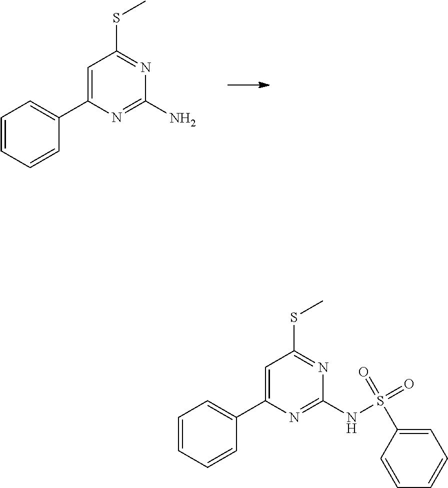 [SMILES: CSc1cc(-c2ccccc2)nc(N)n1 ; CSc1cc(-c2ccccc2)nc(NS(=O)(=O)c2ccccc2)n1]

To a solution of 4-methylsulfanyl-6-phenyl-pyrimidin-2-amine (1.5 g, 6.903 mmol) in DMF (15 mL) at 0° C. was added sodium hydride (approximately 1.104 g of 60% w/w, 27.61 mmol) and the reaction was stirred at room temperture for 20 minutes. It was cooled back to 0° C. and benzenesulfonyl chloride (approximately 1.828 g, 1.321 mL, 10.35 mmol) in DMF (4 mL) was added and the reaction was stirred at room temperature for 30 minutes. The reaction mixture was poured into ice and was extracted with ethyl acetate. The aqueous layer was neutralized with 2 N HCl (approximately 3 mL) and was extracted with ethyl acetate. The combined organic layer was washed with water (2-3 times) and was separated, dried over Na₂SO₄, concentrated and the residue was dissolved in acetonitrile and partitioned between hexane and acetonitrile. The acetonitrile layer was separated, concentrated and the residue was sonicated with ether, the precipitate was filtered to collect part of the product. The filtrate was purified by silica gel chromatography using 0-50% ethyl acetate in hexane to afford a combined total of N-(4-methylsulfanyl-6-phenyl-pyrimidin-2-yl)benzenesulfonamide (1.3 g, 53%). ESI-MS m/z calc. 357.06058, found 358.2 (M+1)⁺; Retention time: 0.66 minutes; LC method A.

### Step 4: N-(4-methylsulfonyl-6-phenyl-pyrimidin-2-yl)benzenesulfonamide

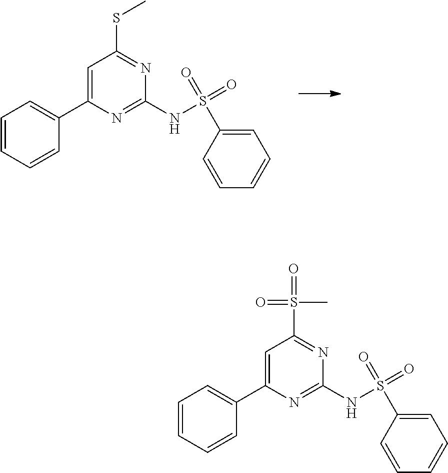 [SMILES: CSc1cc(-c2ccccc2)nc(NS(=O)(=O)c2ccccc2)n1 ; CS(=O)(=O)c1cc(-c2ccccc2)nc(NS(=O)(=O)c2ccccc2)n1]

To a solution of N-(4-methylsulfanyl-6-phenyl-pyrimidin-2-yl)benzenesulfonamide (1.4 g, 3.917 mmol) in methylene chloride (15 mL) was added 3-chloroperoxybenzoic acid (approximately 1.352 g, 7.834 mmol) at 0° C. The reaction mixture was stirred at room temperature for 30 minutes. Another 1 eq. of m-chloroperoxy benzoic acid was added and the reaction was stirred for 30 minutes. The reaction mixture was diluted with sodium thiosulfate and was stirred for 10 minutes. To this mixture was added ethyl acetate. The organic layer was dried over Na₂SO₄, concentrated and the residue was recrystallized using a ethyl acetate/hexane mixture to afford N-(4-methylsulfonyl-6-phenyl-pyrimidin-2-yl)benzenesulfonamide (1.1 g, 72%) as a white solid. ESI-MS m/z calc. 389.05038, found 390.2 (M+1)⁺; Retention time: 0.56 minutes; LC method D.

### Step 5: N-[4-[cyano(phenyl)methyl]-6-phenyl-pyrimidin-2-yl]benzenesulfonamide (Compound 146)

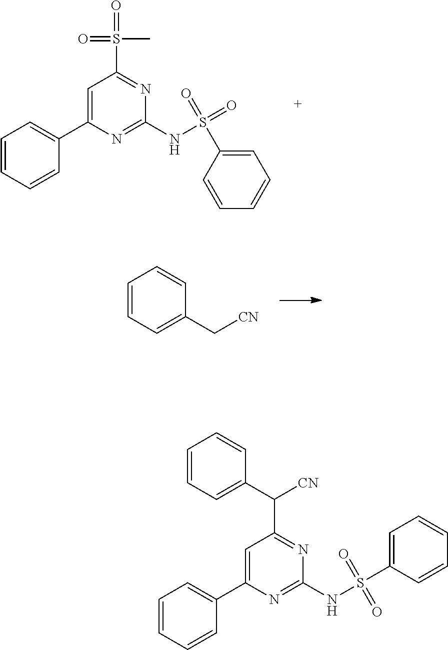 [SMILES: CS(=O)(=O)c1cc(-c2ccccc2)nc(NS(=O)(=O)c2ccccc2)n1 ; N#CCc1ccccc1 ; N#CC(c1ccccc1)c1cc(-c2ccccc2)nc(NS(=O)(=O)c2ccccc2)n1]

To a solution of 2-phenylacetonitrile (approximately 18.05 mg, 17.78 μL, 0.1541 mmol) in DMF (1 mL) was added NaH (approximately 7.703 mg of 60% w/w, 0.1926 mmol) at 0° C. It was stirred for 20 min at rt. To this was added N-(4-methylsulfonyl-6-phenyl-pyrimidin-2-yl)benzenesulfonamide (15 mg, 0.03852 mmol) at 0° C. The reaction mixture was stirred at room temperature for 30 minutes. It was neutralized with 2 drops of water. The reaction mixture was filtered, diluted with DMSO and was purified by reverse phase HPLC using 1-99% acetonitrile in water using HCl as modifier to afford N-[4-[cyano(phenyl)methyl]-6-phenyl-pyrimidin-2-yl]benzenesulfonamide (2 mg). ESI-MS m/z calc. 426.11505, found 427.3 (M+1)⁺; Retention time: 7.41 minutes (LC method L).

### Step 6: N-(4-benzoyl-6-phenyl-pyrimidin-2-yl)benzenesulfonamide (Compound 1109)

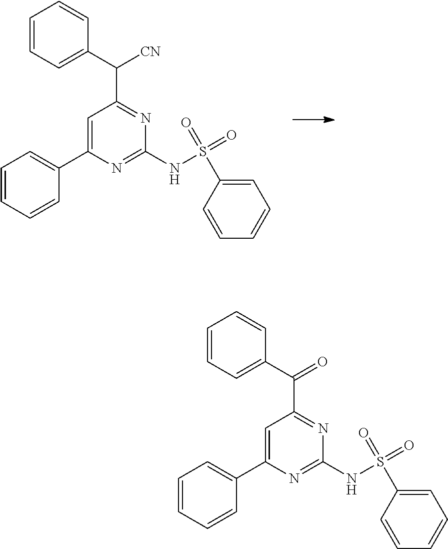 [SMILES: N#CC(c1ccccc1)c1cc(-c2ccccc2)nc(NS(=O)(=O)c2ccccc2)n1 ; O=C(c1ccccc1)c1cc(-c2ccccc2)nc(NS(=O)(=O)c2ccccc2)n1]

To a solution of N-[4-[cyano(phenyl)methyl]-6-phenyl-pyrimidin-2-yl]benzenesulfonamide (10 mg, 0.02345 mmol) in dioxane (500 μL) and water (500 μL) was added potassium permanganate (approximately 7.412 mg, 0.04690 mmol) and the reaction was heated at 100° C. for 30 minutes. The reaction mixture was filtered, and the filtrate was concentrated. The residue was dissolved in DMSO and was purified by reverse phase HPLC using 1-99% acetonitrile in water using HCl as modifier to afford N-(4-benzoyl-6-phenyl-pyrimidin-2-yl)benzenesulfonamide which was used for next step. ESI-MS m/z calc. 415.09906, found 416.43 (M+1)⁺; Retention time: 1.88 minutes; LC method F. ¹H NMR (400 MHz, Chloroform-d) δ 8.15-8.09 (m, 2H), 8.09-8.03 (m, 2H), 7.99-7.92 (m, 3H), 7.91 (s, 1H), 7.73 -7.66 (m, 1H), 7.60-7.46 (m, 6H), 7.36-7.29 (m, 2H).

### Step 7: N-[4-[hydroxy(phenyl)methyl]-6-phenyl-pyrimidin-2-yl]benzenesulfonamide (Compound 1110)

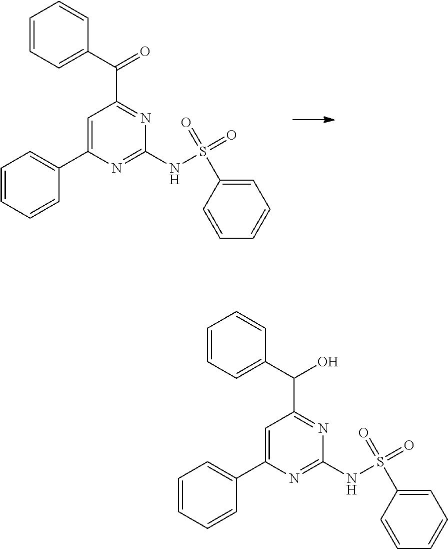 [SMILES: O=C(c1ccccc1)c1cc(-c2ccccc2)nc(NS(=O)(=O)c2ccccc2)n1 ; O=S(=O)(Nc1nc(-c2ccccc2)cc(C(O)c2ccccc2)n1)c1ccccc1]

To a solution of N-(4-benzoyl-6-phenyl-pyrimidin-2-yl)benzenesulfonamide (10 mg, 0.02407 mmol) in MeOH (1 mL) at 0° C. was added sodium borohydride (approximately 2.732 mg, 0.07221 mmol). The reaction mixture was stirred at room temperature for 20 minutes. It was quenched with 2 drops of water, filtered and purified by reverse phase HPLC using 1-99% acetonitrile in water using HCl as modifier to afford N-[4-[hydroxy(phenyl)methyl]-6-phenyl-pyrimidin-2-yl]benzenesulfonamide. ESI-MS m/z calc. 417.11472, found 418.46 (M+1)⁺; Retention time: 1.66 minutes; LC method F.

### Example 319: Preparation of Compound 1111

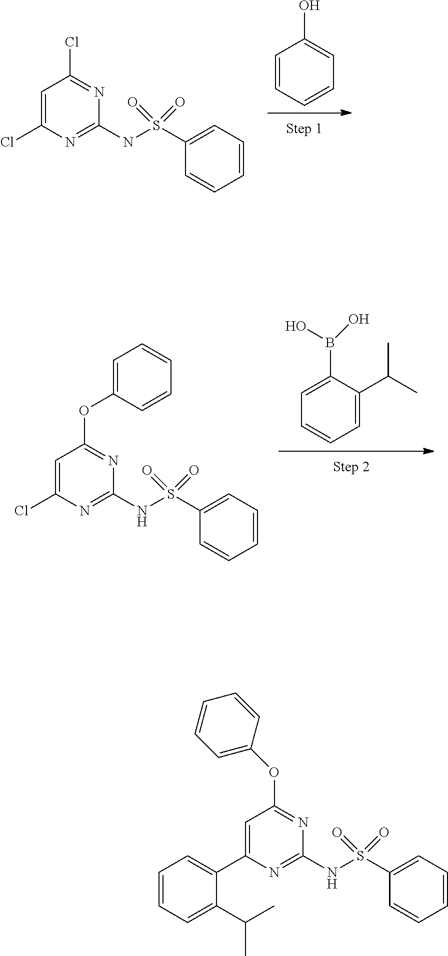 [SMILES: O=S(=O)(Nc1nc(Cl)cc(Cl)n1)c1ccccc1 ; Oc1ccccc1 ; O=S(=O)(Nc1nc(Cl)cc(Oc2ccccc2)n1)c1ccccc1 ; CC(C)c1ccccc1B(O)O ; CC(C)c1ccccc1-c1cc(Oc2ccccc2)nc(NS(=O)(=O)c2ccccc2)n1]

### Step 1: N-(4-chloro-6-phenoxy-pyrimidin-2-yl)benzenesulfonamide

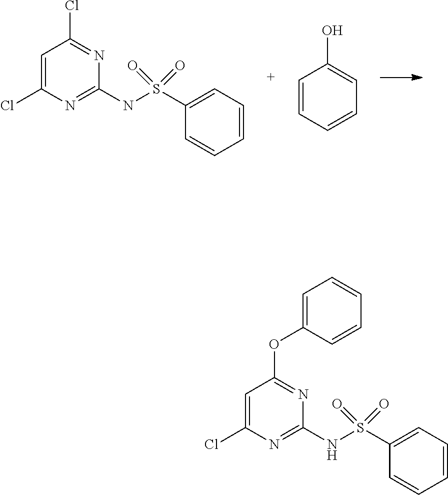 [SMILES: O=S(=O)(Nc1nc(Cl)cc(Cl)n1)c1ccccc1 ; Oc1ccccc1 ; O=S(=O)(Nc1nc(Cl)cc(Oc2ccccc2)n1)c1ccccc1]

To a solution of N-(4,6-dichloropyrimidin-2-yl)benzenesulfonamide (5 g, 16.44 mmol) in DMF (50.00 mL) was added phenol (approximately 1.702 g, 1.606 mL, 18.08 mmol) followed potassium carbonate (approximately 6.816 g, 49.32 mmol). The mixture was heated at 110° C. overnight. The reaction mixture was cooled down with ice, carefully acidified with 1 N HCl at −0° C. and was extracted with ethyl acetate. The organic layer was washed with brine, dried over Na₂SO₄, concentrated and the residue was purified by silica gel chromatography using a gradient of ethyl acetate/hexane to give N-(4-chloro-6-phenoxy-pyrimidin-2-yl)benzenesulfonamide (4.565 g, 60%)¹H NMR (400 MHz, DMSO) δ 12.10 (s, 1H), 7.56 (dd, J=16.0, 8.3 Hz, 3H), 7.47-7.38 (m, 5H), 7.20 (d, J=7.6 Hz, 2H), 6.87 (s, 1H).

### Step 2: N-[4-(2-isopropylphenyl)-6-phenoxy-pyrimidin-2-yl]benzenesulfonamide

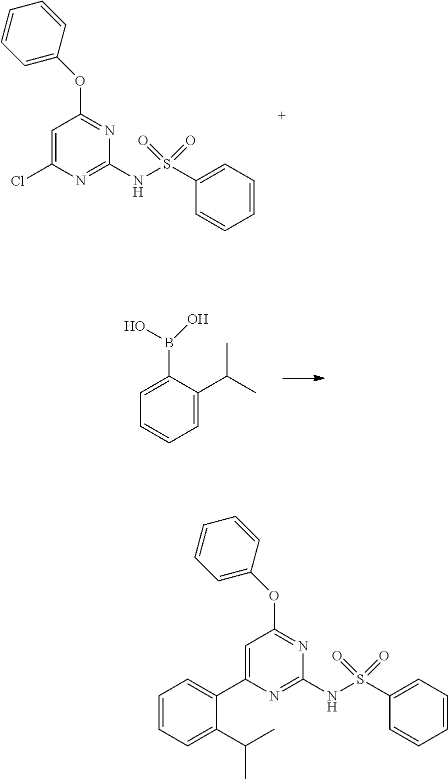 [SMILES: O=S(=O)(Nc1nc(Cl)cc(Oc2ccccc2)n1)c1ccccc1 ; CC(C)c1ccccc1B(O)O ; CC(C)c1ccccc1-c1cc(Oc2ccccc2)nc(NS(=O)(=O)c2ccccc2)n1]

To a mixture of N-(4-chloro-6-phenoxy-pyrimidin-2-yl)benzenesulfonamide (25 mg, 0.06910 mmol), (2-isopropylphenyl)boronic acid (approximately 16.99 mg, 0.1036 mmol), potassium carbonate (approximately 138.2 μL of 2 M, 0.2764 mmol) in NMP (1 mL) was added Pd(dppf)Cl₂ (approximately 28.21 mg, 0.03455 mmol). The reaction mixture was flushed with nitrogen multiple times. The mixture was heated at 100° C. overnight. It was filtered and was purified by reverse phase HPLC using 1-99% acetonitrile in water and using HCl as modifier to afford N-[4-(2-isopropylphenyl)-6-phenoxy-pyrimidin-2-yl]benzenesulfonamide (8.5 mg, 28%). ESI-MS m/z calc. 445.14603, found 446.54 (M+1)⁺; Retention time: 2.11 minutes; LC method A.

### Example 320: Preparation of Compound 370

### Step 1: N-(4,6-Diphenoxypyrimidin-2-yl)benzenesulfonamide

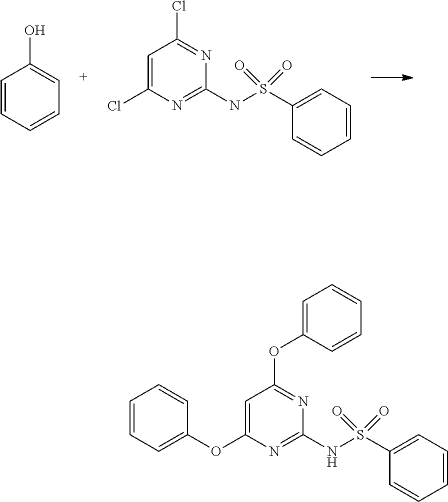 [SMILES: Oc1ccccc1 ; O=S(=O)(Nc1nc(Cl)cc(Cl)n1)c1ccccc1 ; O=S(=O)(Nc1nc(Oc2ccccc2)cc(Oc2ccccc2)n1)c1ccccc1]

To a solution of N-(4,6-dichloropyrimidin-2-yl)benzenesulfonamide (20 mg, 0.06576 mmol) in NMP (750 μL) was added phenol (approximately 24.75 mg, 23.35 μL, 0.2630 mmol) followed by potassium carbonate (approximately 45.44 mg, 0.3288 mmol) and the reaction was heated in a microwave for 30 minutes at 180° C. The reaction mixture was filtered, diluted with DMSO and purified by reverse phase HPLC using a 1-99% gradient of acetonitrile in water (HCl modifier) to afford N-(4,6-diphenoxypyrimidin-2-yl)benzenesulfonamide ESI-MS m z calc. 419.09396, found 420.25 (M+1)⁺; Retention time: 1.97 minutes (LC method F).

### Example 321: Preparation of Compound 1112

### Step 1: N-[4-phenoxy-6-(2,4,6-trimethylphenyl)pyrimidin-2-yl]benzenesulfonamide

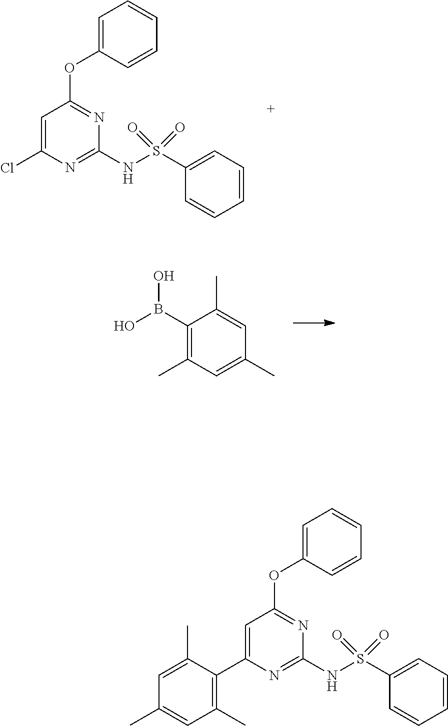 [SMILES: O=S(=O)(Nc1nc(Cl)cc(Oc2ccccc2)n1)c1ccccc1 ; Cc1cc(C)c(B(O)O)c(C)c1 ; Cc1cc(C)c(-c2cc(Oc3ccccc3)nc(NS(=O)(=O)c3ccccc3)n2)c(C)c1]

To a mixture of N-(4-chloro-6-phenoxy-pyrimidin-2-yl)benzenesulfonamide (20 mg, 0.05528 mmol), (2,4,6-trimethylphenyl)boronic acid (approximately 18.14 mg, 0.1106 mmol), in DMSO (500 μL) was added sodium carbonate (approximately 165.8 μL of 1 M, 0.1658 mmol) and Pd(dppf)C₁₂ (approximately 9.032 mg, 0.01106 mmol). The mixture was thoroughly flushed with nitrogen. It was heated in microwave at 150° C. for 3 minutes. The reaction mixture was filtered, diluted with DMSO and was purified by reverse phase HPLC using 1-99% acetonitrile in water using HCl as modifier to afford N-[4-phenoxy-6-(2,4,6-trimethylphenyl)pyrimidin-2-yl]benzenesulfonamide. ESI-MS m/z calc. 445.14603, found 446.33 (M+1)⁺; Retention time: 1.77 minutes (LC method F).

### Example 322: Preparation of Compound 1113

### Step 1: N-[4-(2,6-Diisopropylphenyl)-6-phenoxy-pyrimidin-2-yl]benzenesulfonamide

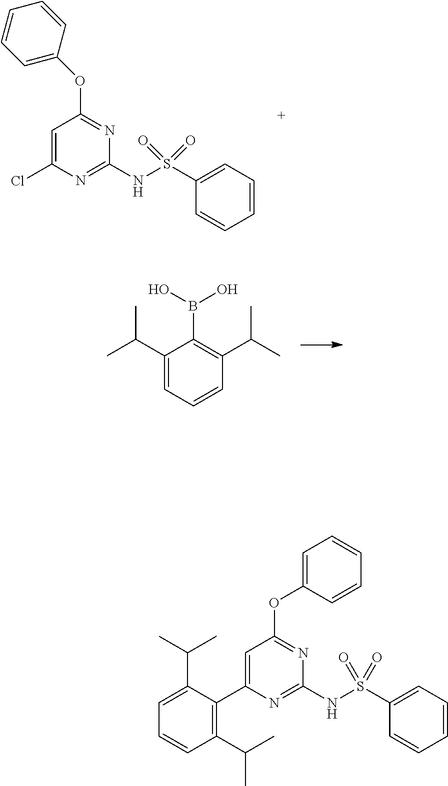 [SMILES: O=S(=O)(Nc1nc(Cl)cc(Oc2ccccc2)n1)c1ccccc1 ; CC(C)c1cccc(C(C)C)c1B(O)O ; CC(C)c1cccc(C(C)C)c1-c1cc(Oc2ccccc2)nc(NS(=O)(=O)c2ccccc2)n1]

A heterogeneous mixture of N-(4-chloro-6-phenoxy-pyrimidin-2-yl)benzenesulfonamide (25.0 mg, 0.0553 mmol), (1,3-Bis(2,6-diisopropylphenyl)imidazolidene) (3-chloropyridyl) palladium(II) dichloride (3.8 mg, 0.005568 mmol), (2,6-diisopropylphenyl)boronic acid (34.2 mg, 0.1659 mmol), and potassium tert-butoxide (24.8 mg, 0.2210 mmol) in tert-butanol (220 μL) was microwaved for 30 minutes at 65° C. in a sealed pressure tube. The reaction mixture was diluted with DMSO (0.5 mL) and filtered through a 0.45 μM PTFE syringe filter. The crude solution was separated by HPLC (gradient: 1 to 99% acetonitrile in water with 0.1% hydrochloric acid) to afford N-[4-(2,6-diisopropylphenyl)-6-phenoxy-pyrimidin-2-yl]benzenesulfonamide (13.2 mg, 49%) as a white solid. ESI-MS m/z calc. 487.19296, found 488.53 (M+1)⁺; Retention time: 2.29 minutes (LC method A).

### Example 323: Preparation of Compound 1114

### Step 1: N-[4-(2,6-Dimethylphenyl)-6-phenoxy-pyrimidin-2-yl]benzenesulfonamide

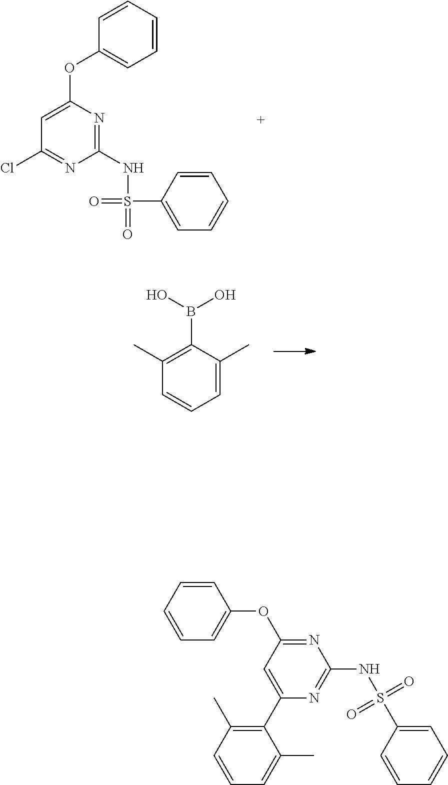

To a mixture of N-(4-chloro-6-phenoxy-pyrimidin-2-yl)benzenesulfonamide (20 mg, 0.05528 mmol), (2,6-dimethylphenyl)boronic acid (16.5 mg, 0.11 mmol), in DMF (500 μL) was added sodium carbonate (approximately 110.6 μL of 2 M, 0.2211 mmol), Pd(ddpf) C₁₂ (approximately 8.093 mg, 0.01106 mmol). The mixture was thoroughly flushed with nitrogen and heated at 100° C. for 1 hour. The reaction mixture was filtered and purified by reverse phase HPLC using 30-99% acetonitrile in water using HCl as modifier. N-[4-(2,6-dimethylphenyl)-6-phenoxy-pyrimidin-2-yl]benzenesulfonamide (5.2 mg, 21%). ESI-MS m/z calc. 431.13037, found 431.0 (M+1)⁺; Retention time: 1.89 minutes; LC method A.

### Example 324: Preparation of Compound 1115

### Step 1: N-[4-(4-isopropylphenyl)-6-phenoxy-pyrimidin-2-yl]benzenesulfonamide

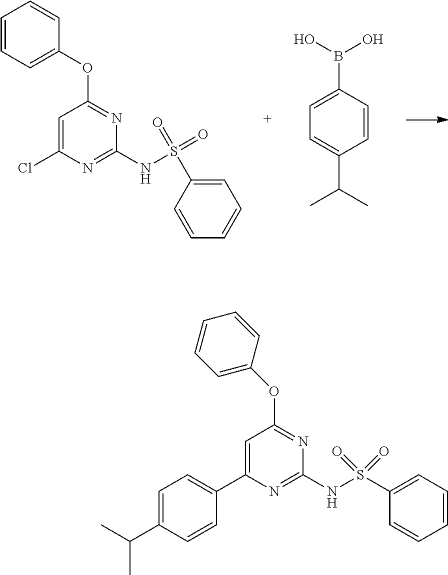 [SMILES: O=S(=O)(Nc1nc(Cl)cc(Oc2ccccc2)n1)c1ccccc1 ; CC(C)c1ccc(B(O)O)cc1 ; CC(C)c1ccc(-c2cc(Oc3ccccc3)nc(NS(=O)(=O)c3ccccc3)n2)cc1]

To a mixture of N-(4-chloro-6-phenoxy-pyrimidin-2-yl)benzenesulfonamide (25 mg, 0.06910 mmol), (4-isopropylphenyl)boronic acid (approximately 16.99 mg, 0.1036 mmol), potassium carbonate (approximately 138.2 μL of 2 M, 0.2764 mmol) in NMP (1 mL) was added Pd(dppf)Cl₂ (approximately 28.21 mg, 0.03455 mmol). The reaction mixture was flushed with nitrogen multiple times. The mixture was heated at 100° C. overnight. It was filtered and was purified by reverse phase HPLC using 1-99% acetonitrile in water and using HCl as modifier to afford N-[4-(4-isopropylphenyl)-6-phenoxy-pyrimidin-2-yl]benzenesulfonamide (13.2 mg, 43%). ESI-MS m/z calc. 445.14603, found 446.32 (M+1)⁺; Retention time: 1.58 minutes; LC method E.

### Example 325: Preparation of Compound 371

### Step 1: N-[4-(5,5-Dimethyl-2-oxo-1-piperidyl)-6-phenoxy-pyrimidin-2-yl]benzenesulfonamide

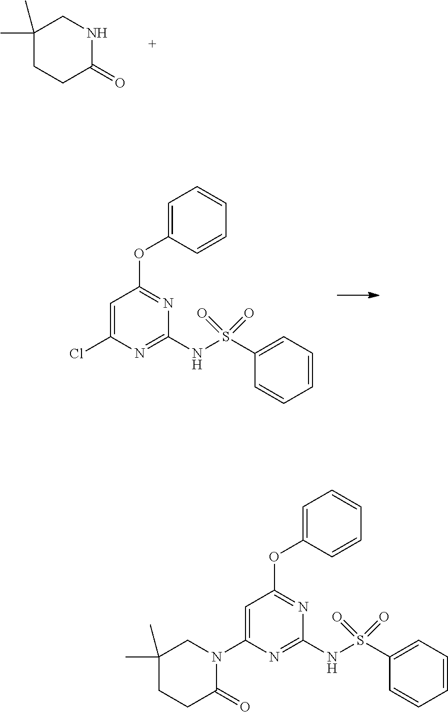

A mixture of N-(4-chloro-6-phenoxy-pyrimidin-2-yl)benzenesulfonamide (20 mg, 0.05528 mmol), 5,5-dimethylpiperidin-2-one (approximately 28.12 mg, 0.2211 mmol), Xantphos (20 mg, 0.03457 mmol), Pd(OAc)₂ (4 mg, 0.01782 mmol) and Cs₂CO₃ (approximately 54.02 mg, 0.1658 mmol) in dioxane (0.5 mL) was degassed with a stream of nitrogen for 5 minutes before heating to 120° C. in a sealed tube for 15 minutes. The mixture was diluted with MeOH, filtered and purified by HPLC (1-99% ACN in water (HCl modifier)) to give N-[4-(5,5-dimethyl-2-oxo-1-piperidyl)-6-phenoxy-pyrimidin-2-yl]benzenesulfonamide (14.5 mg, 58%) as a glass. ESI-MS m/z calc. 452.15182, found 453.4 (M+1)⁺; Retention time: 1.77 minutes (LC method A).

### Example 326: Preparation of Compound 1116

### Step 1: N-[4-[2-(dimethylamino)phenyl]-6-phenoxy-pyrimidin-2-yl]benzenesulfonamide

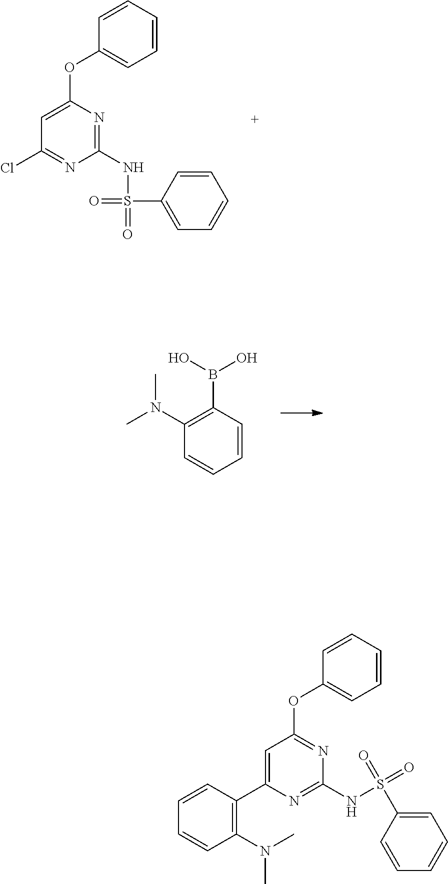 [SMILES: O=S(=O)(Nc1nc(Cl)cc(Oc2ccccc2)n1)c1ccccc1 ; CN(C)c1ccccc1B(O)O ; CN(C)c1ccccc1-c1cc(Oc2ccccc2)nc(NS(=O)(=O)c2ccccc2)n1]

The compound was prepared in a manner analogous to that described above using commercially available [2-(dimethylamino)phenyl]boronic acid to give N-[4-[2-(dimethylamino)phenyl]-6-phenoxy-pyrimidin-2-yl]benzenesulfonamide (6 mg, 40%). ESI-MS m/z calc. 446.14127, found 447.0 (M+1)⁺; Retention time: 1.19 minutes; LC method A. ¹H NMR (400 MHz, DMSO) δ 12.90 (d, J=333.6 Hz, 1H), 8.18 (s, 1H), 7.74 (s, 1H), 7.60 (t, J=7.6 Hz, 3H), 7.55-7.32 (m, 6H), 7.24 (d, J=8.3 Hz, 2H), 3.21 (s, 6H).

### Example 327: Preparation of Compound 1117

### Step 1: N-[4-(2-isopropoxyphenyl)-6-phenoxy-pyrimidin-2-yl]benzenesulfonamide

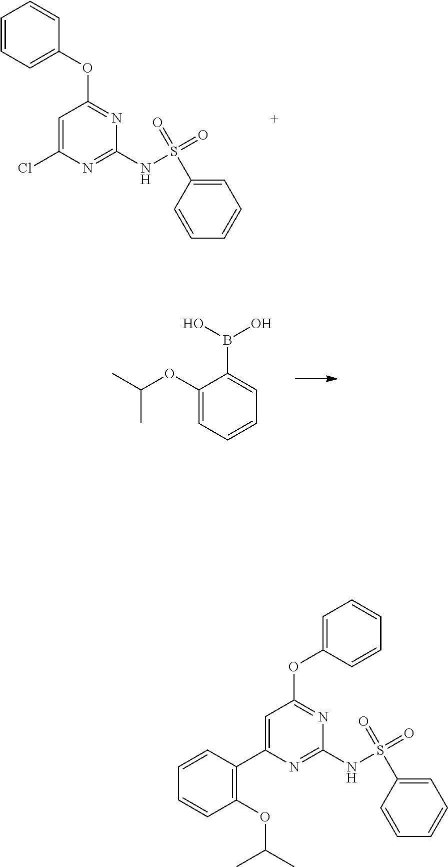 [SMILES: O=S(=O)(Nc1nc(Cl)cc(Oc2ccccc2)n1)c1ccccc1 ; CC(C)Oc1ccccc1B(O)O ; CC(C)Oc1ccccc1-c1cc(Oc2ccccc2)nc(NS(=O)(=O)c2ccccc2)n1]

The compound was prepared in a manner analogous to that described above using commercially available (2-isopropoxyphenyl)boronic acid to give N-[4-(2-isopropoxyphenyl)-6-phenoxy-pyrimidin-2-yl]benzenesulfonamide (10.2 mg, 33%) as a white solid. ESI-MS m/z calc. 461.14093, found 462.1 (M+1)⁺; Retention time: 2.0 minutes

### Example 328: Preparation of Compound 1118 and Compound 1119

### Step 1: N-[4-phenoxy-6-[3-(trifluoromethyl)pyrazol-1-yl]pyrimidin-2-yl]benzenesulfonamide, Compound 1118, and N-[4,6-bis[3-(trifluoromethyl)pyrazol-1-yl]pyrimidin-2-yl]benzenesulfonamide, Compound 1119

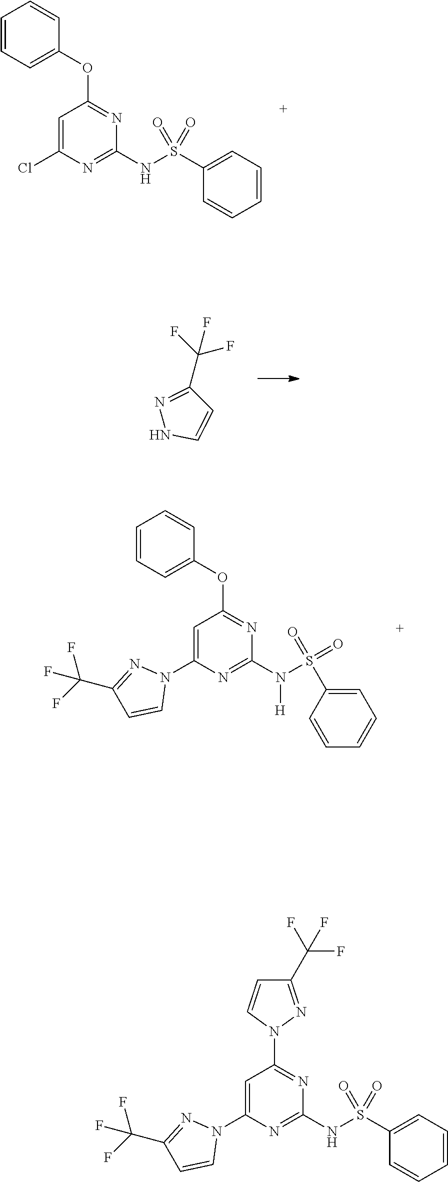 [SMILES: O=S(=O)(Nc1nc(Cl)cc(Oc2ccccc2)n1)c1ccccc1 ; FC(F)(F)c1cc[nH]n1 ; O=S(=O)(Nc1nc(Oc2ccccc2)cc(-n2ccc(C(F)(F)F)n2)n1)c1ccccc1 ; O=S(=O)(Nc1nc(-n2ccc(C(F)(F)F)n2)cc(-n2ccc(C(F)(F)F)n2)n1)c1ccccc1]

To a solution of N-(4-chloro-6-phenoxy-pyrimidin-2-yl)benzenesulfonamide (27.14 mg, 0.075 mmol) and at 0° C. was added NaH (approximately 7.199 mg, 0.3000 mmol) and the reaction mixture stirred at this temp for 10 minutes. The cooling bath was removed, and the reaction mixture stirred at room temperature for 30 minutes then at 80° C. for 1 hour. The reaction mixture was quenched with 0.45 mL of MeOH followed by AcOH (approximately 45.04 mg, 42.65 μL, 0.7500 mmol) which gave a clear solution. The solution was filtered and purified by HPLC (1-99% ACN in water (HCl modifier)). The tubes with the desired product were diluted with brine and the layers separated. The organic layer was dried over MgSO₄, filtered and evaporated to dryness to give both N-[4-phenoxy-6-[3-(trifluoromethyl)pyrazol-1-yl]pyrimidin-2-yl]benzenesulfonamide (4.3 mg, 25%) (ESI-MS m/z calc. 461.07693, found 462.3 (M+1)⁺; Retention time: 2.01 minutes) and N-[4,6-bis[3-(trifluoromethyl)pyrazol-1-yl]pyrimidin-2-yl]benzenesulfonamide (3.0 mg, 16%) (ESI-MS m/z calc. 503.0599, found 504.3 (M+1)⁺; Retention time: 2.08 minutes).

### Example 329: Preparation of Compound 1120

### Step 1: N-[4-(6-methoxy-2-pyridyl)-6-phenoxy-pyrimidin-2-yl]benzenesulfonamide

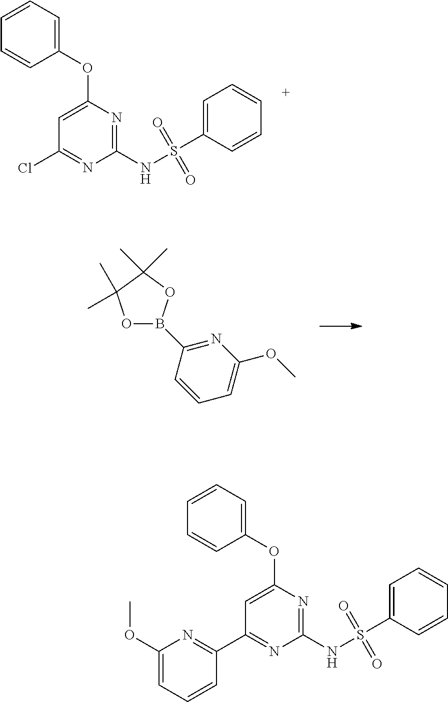

To a mixture of N-(4-chloro-6-phenoxy-pyrimidin-2-yl)benzenesulfonamide (24.96 mg, 0.069 mmol), 2-methoxy-6-(4,4,5,5-tetramethyl-1,3,2-dioxaborolan-2-yl)pyridine (approximately 16.22 mg, 0.06900 mmol), and potassium carbonate (approximately 138.0 μL of 2 M, 0.2760 mmol) in NMP (500 μL) and IPA (500 μL) (1:1) was added (1,3-Bis(2,6-diisopropylphenyl)imidazolidene) (3-chloropyridyl) palladium(II) dichloride (approximately 2.402 mg, 0.003519 mmol). The reaction mixture was heated at 100° C. overnight. It was filtered and was purified by reverse phase HPLC using to afford N-[4-(6-methoxy-2-pyridyl)-6-phenoxy-pyrimidin-2-yl]benzenesulfonamide (2.4 mg). ESI-MS m/z calc. 434.1049, found 435.27 (M+1)⁺; Retention time: 2.12 minutes; LC method A.

### Example 330: Preparation of Compound 1121

### Step 1: N-[4-[2-isopropoxy-5-(trifluoromethyl)-3-pyridyl]-6-phenoxy-pyrimidin-2-yl]benzenesulfonamide

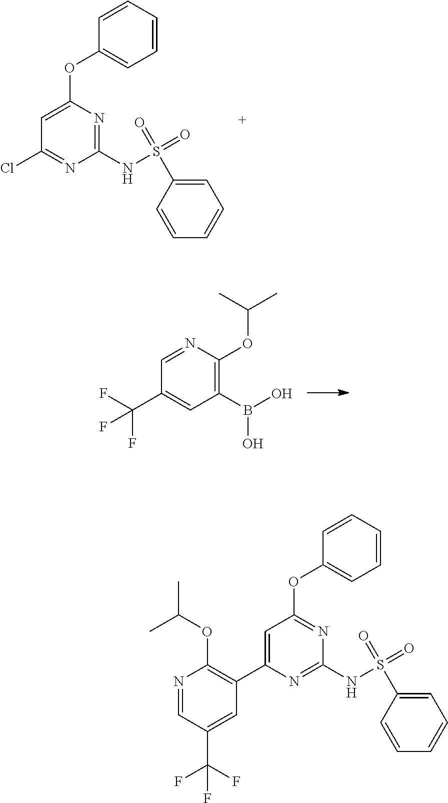 [SMILES: O=S(=O)(Nc1nc(Cl)cc(Oc2ccccc2)n1)c1ccccc1 ; CC(C)Oc1ncc(C(F)(F)F)cc1B(O)O ; CC(C)Oc1ncc(C(F)(F)F)cc1-c1cc(Oc2ccccc2)nc(NS(=O)(=O)c2ccccc2)n1]

The compound was prepared in a manner analogous to that described above using commercially available [2-isopropoxy-5-(trifluoromethyl)-3-pyridyl]boronic acid to give N-[4-[2-isopropoxy-5-(trifluoromethyl)-3-pyridyl]-6-phenoxy-pyrimidin-2-yl]benzenesulfonamide (14.4 mg, 75%). ESI-MS m/z calc. 530.12354, found 531.0 (M+1)⁺; Retention time: 2.28 minutes; LC method A. ¹H NMR (400 MHz, DMSO) δ 11.98 (s, 1H), 8.71 (s, 1H), 8.56 (s, 1H), 7.77 (d, J=7.9 Hz, 2H), 7.63-7.46 (m, 5H), 7.38 (t, J=7.9 Hz, 1H), 7.28 (d, J=8.5 Hz, 2H), 7.23 (s, 1H), 5.42 (dt, J=12.2, 6.1 Hz, 1H), 1.20 (d, J=6.2 Hz, 6H).

### Example 331: Preparation of Compound 1122

### Step 1: N-[4-(2-ethox-4-methyl-3-pyridyl)-6-phenoxy-pyrimidin-2-yl]benzenesulfonamide

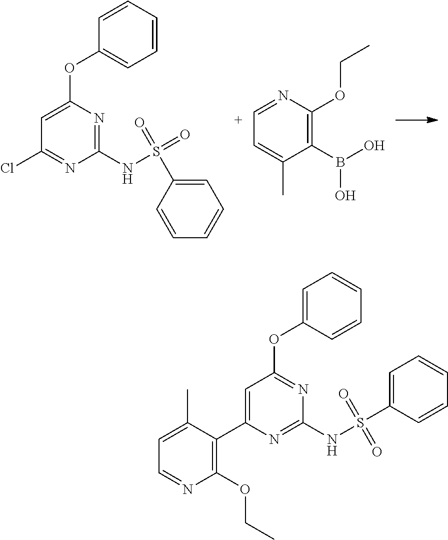 [SMILES: O=S(=O)(Nc1nc(Cl)cc(Oc2ccccc2)n1)c1ccccc1 ; CCOc1nccc(C)c1B(O)O ; CCOc1nccc(C)c1-c1cc(Oc2ccccc2)nc(NS(=O)(=O)c2ccccc2)n1]

The compound was prepared in a manner analogous to that described above using commercially available (2-ethox-4-methyl-3-pyridyl)boronic acid to give N-[4-(2-ethox-4 -methyl-3-pyridyl)-6-phenoxy-pyrimidin-2-yl]benzenesulfonamide (17.1 mg, 48%). ¹H NMR (400 MHz, DMSO) δ 11.77 (s, 1H), 8.09 (d, J=5.2 Hz, 1H), 7.57 (t, J=7.7 Hz, 3H), 7.51-7.31 (m, 5H), 7.25 (d, J=7.7 Hz, 2H), 6.95 (d, J=5.2 Hz, 1H), 6.66 (s, 1H), 4.27 (q, J=7.0 Hz, 2H), 2.06 (s, 3H), 1.16 (s, 3H). ESI-MS m/z calc. 462.13617, found 463.0 (M+1)⁺; Retention time: 1.8 minutes (LC method A).

### Example 332: Preparation of Compound 1123

### Step 1: N-[4-(3,3-Dimethylbut-1-ynyl)-6-phenoxy-pyrimidin-2-yl]benzenesulfonamide

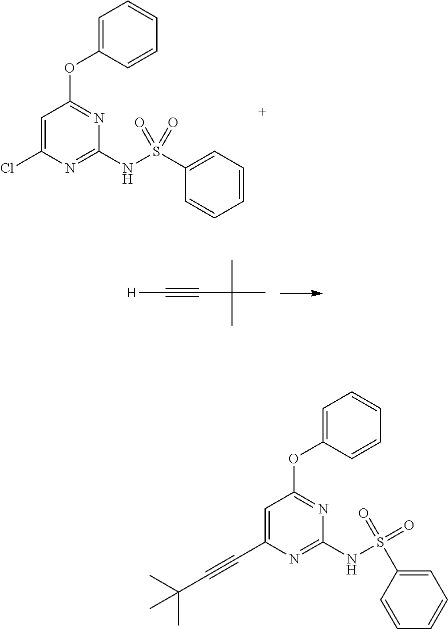

A solution of N-(4-chloro-6-phenoxy-pyrimidin-2-yl)benzenesulfonamide (30.66 mg, 0.075 mmol), 3,3-dimethylbut-1-yne (approximately 18.48 mg, 26.86 μL, 0.2250 mmol) in DMF (0.2 mL) was degassed by bubbling nitrogen through the solution for 10 minutes, at this time dichloropalladium;triphenylphosphine (5.2 mg, 0.007 mmol) and CuI (1.4 mg, 0.007 mmo) and TEA (104 μL, 0.75 mmol) were added and the reaction mixture was stirred at 80° C. for 16 hours. The reaction mixture was diluted with MeOH, filtered and purification by HPLC (1-99% ACN in water (HCl modifier)) gave N-[4-(3,3-dimethylbut-1-ynyl)-6-phenoxy-pyrimidin-2-yl]benzenesulfonamide (4.4 mg) as a glass. ESI-MS m/z calc. 407.13037, found 408.3 (M+1)⁺; Retention time: 2.01 minutes, LC method A.

### Example 333: Preparation of Compound 1124

### Step 1: N-[4-(4,4-Dimethylcyclohexen-1-yl)-6-phenoxy-pyrimidin-2-yl]benzenesulfonamide

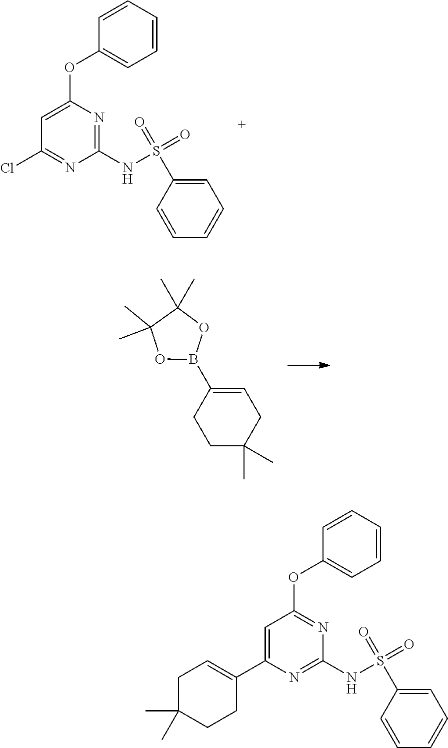

The compound was prepared in a manner analogous to that described above using commercially available 2-(4,4-dimethylcyclohexen-1-yl)-4,4,5,5-tetramethyl-1,3,2-dioxaborolane to give N-[4-(4,4-dimethylcyclohexen-1-yl)-6-phenoxy-pyrimidin-2-yl]benzenesulfonamide (5 mg, 56%). ESI-MS m/z calc. 435.16165, found 436.0 (M+1)⁺; Retention time: 2.19 minutes; LC method A. ¹H NMR (400 MHz, DMSO) δ 11.52 (s, 1H), 7.61 (s, 3H), 7.50 (t, J=7.7 Hz, 2H), 7.42 (d, J=6.1 Hz, 2H), 7.33 (t, J=7.1 Hz, 1H), 7.17 (d, J=8.3 Hz, 2H), 6.80 (s, 1H), 6.59 (s, 1H), 2.25 (s, 2H), 2.00 (s, 2H), 1.42 (t, J=6.3 Hz, 2H), 1.17 (s, 2H), 0.89 (s, 6H).

### Example 334: Preparation of Compound 1125

### Step 1: N-[4-(4,4-Dimethylcyclohexyl)-6-phenoxy-pyrimidin-2-yl]benzenesulfonamide

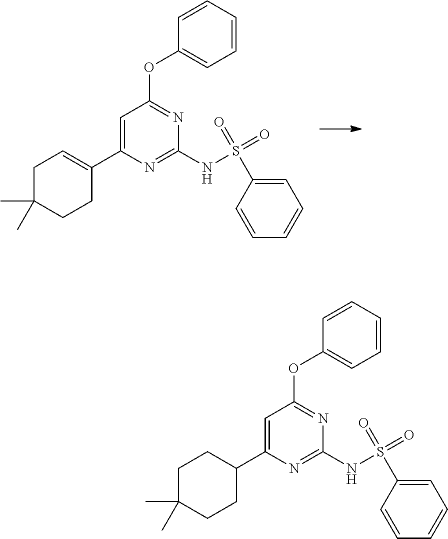 [SMILES: CC1(C)CC=C(c2cc(Oc3ccccc3)nc(NS(=O)(=O)c3ccccc3)n2)CC1 ; CC1(C)CCC(c2cc(Oc3ccccc3)nc(NS(=O)(=O)c3ccccc3)n2)CC1]

Pd/C (10 mg of 5% w/w, 0.004698 mmol) was added to N-[4-(4,4-dimethylcyclohexen-1-yl)-6-phenoxy-pyrimidin-2-yl]benzenesulfonamide (20 mg, 0.04592 mmol) in methanol (10 mL). The flask was purged with nitrogen and stirred under a balloon of hydrogen at room temperature for 3 hours. The reaction mixture was filtered, concentrated under reduced pressure. The crude was suspended in DMSO, filtered and purified on reverse phase HPLC (HCl modifier, 30-99% ACN-H₂O) to give product N-[4-(4,4-dimethylcyclohexyl)-6-phenoxy-pyrimidin-2-yl]benzenesulfonamide (3.5 mg). ESI-MS m/z calc. 437.1773, found 438.0 (M+1)⁺; Retention time: 2.14 minutes; LC method A. ¹H NMR (400 MHz, DMSO) δ 11.59 (s, 1H), 7.66-7.47 (m, 6H), 7.45-7.31 (m, 3H), 7.17 (d, J=8.1 Hz, 2H), 6.49 (s, 1H), 1.56 (s, 3H), 1.41 (d, J=12.8 Hz, 2H), 1.23 (s, 3H), 0.93 (d, J=10.3 Hz, 7H).

### Example 335: Preparation of Compound 1126 and Compound 1127

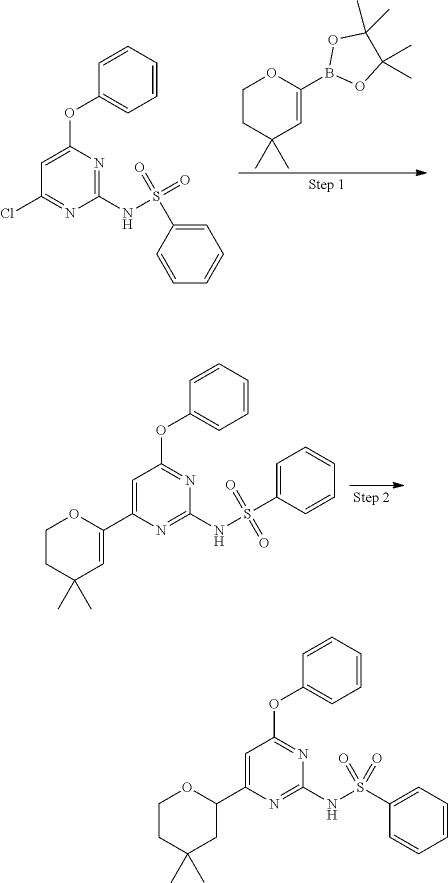 [SMILES: O=S(=O)(Nc1nc(Cl)cc(Oc2ccccc2)n1)c1ccccc1 ; CC1(C)C=C(B2OC(C)(C)C(C)(C)O2)OCC1 ; CC1(C)C=C(c2cc(Oc3ccccc3)nc(NS(=O)(=O)c3ccccc3)n2)OCC1 ; CC1(C)CCOC(c2cc(Oc3ccccc3)nc(NS(=O)(=O)c3ccccc3)n2)C1]

### Step 1: N-[4-(4,4-Dimethyl-2,3-dihydropyran-6-yl)-6-phenoxy-pyrimidin-2-yl]benzenesulfonamide

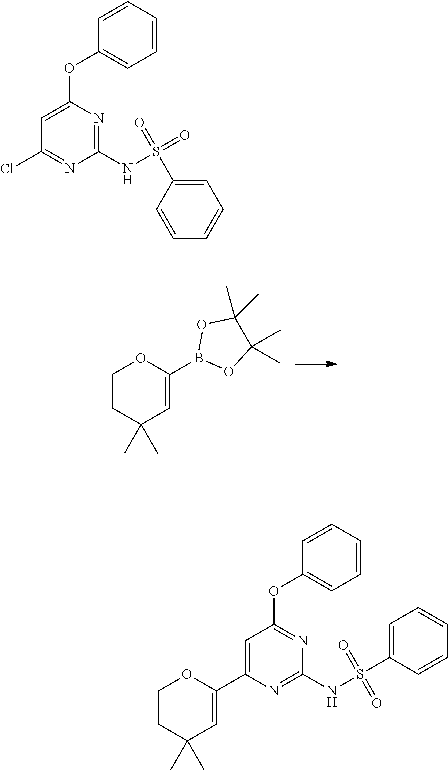 [SMILES: O=S(=O)(Nc1nc(Cl)cc(Oc2ccccc2)n1)c1ccccc1 ; CC1(C)C=C(B2OC(C)(C)C(C)(C)O2)OCC1 ; CC1(C)C=C(c2cc(Oc3ccccc3)nc(NS(=O)(=O)c3ccccc3)n2)OCC1]

The compound was prepared in a manner analogous to that described above using commercially available 2-(4,4-dimethyl-2,3-dihydropyran-6-yl)-4,4,5,5-tetramethyl-1,3,2-dioxaborolane to give N-[4-(4,4-dimethyl-2,3-dihydropyran-6-yl)-6-phenoxy-pyrimidin-2-yl]benzenesulfonamide (6.1 mg, 99%). ESI-MS m/z calc. 437.14093, found 438.0 (M+1)⁺; Retention time: 2.04 minutes; LC method A. ¹H NMR (400 MHz, DMSO) δ 11.66 (s, 1H), 7.72 (d, J=7.6 Hz, 2H), 7.61 (t, J=7.3 Hz, 1H), 7.49 (q, J=7.5 Hz, 4H), 7.34 (t, J=7.4 Hz, 1H), 7.19 (d, J=7.7 Hz, 2H), 6.43 (s, 1H), 5.84 (s, 1H), 4.08-3.99 (m, 2H), 1.65-1.57 (m, 2H), 1.09 (s, 6H).

### Step 2: N-[4-(4,4-dimethyltetrahydropyran-2-yl)-6-phenoxy-pyrimidin-2-yl]benzenesulfonamide

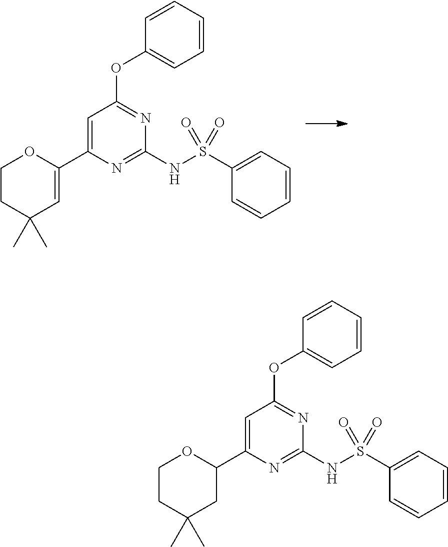 [SMILES: CC1(C)C=C(c2cc(Oc3ccccc3)nc(NS(=O)(=O)c3ccccc3)n2)OCC1 ; CC1(C)CCOC(c2cc(Oc3ccccc3)nc(NS(=O)(=O)c3ccccc3)n2)C1]

Pd/C (10 mg of 5% w/w, 0.004698 mmol) was added to N-[4-(4,4-dimethyl-2,3-dihydropyran-6-yl)-6-phenoxy-pyrimidin-2-yl]benzenesulfonamide (20 mg, 0.04571 mmol) in methanol (10 mL). The flask was purged with nitrogen and stirred under a balloon of hydrogen. The reaction mixture was filtered, concentrated under reduced pressure. The crude was suspended in DMSO, filtered and purified on reverse phase HPLC (HCl modifier, 30-99% ACN-H₂O) to give product N-[4-(4,4-dimethyltetrahydropyran-2-yl)-6-phenoxy-pyrimidin-2-yl]benzenesulfonamide (3.2 mg). ESI-MS m/z calc. 439.1566, found 440.0 (M+1)⁺; Retention time: 1.99 minutes; LC method A.

### Example 336: Preparation of Compound 1128 and Compound 1129

### Step 1: N-[4-(6,6-dimethylcyclohexen-1-yl)-6-phenoxy-pyrimidin-2-yl]benzenesulfonamide

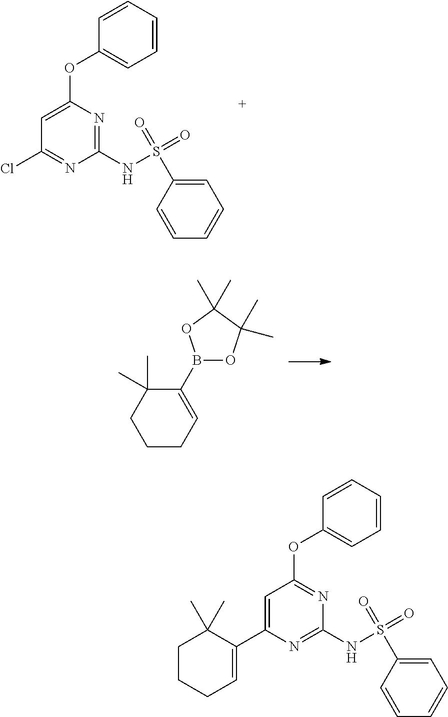

The compound was prepared in a manner analogous to that described above using commercially available 2-(6,6-dimethylcyclohexen-1-yl)-4,4,5,5-tetramethyl-1,3,2-dioxaborolane to give N-[4-(6,6-dimethylcyclohexen-1-yl)-6-phenoxy-pyrimidin-2-yl]benzenesulfonamide (4.0 mg, 49%). ESI-MS m/z calc. 435.16165, found 436.0 (M+1)⁺; Retention time: 2.08 minutes; LC method A. ¹H NMR (400 MHz, DMSO) δ 11.58 (s, 1H), 7.53(dd, J=17.1, 9.4 Hz, 5H), 7.45-7.33 (m, 3H), 7.20 (d, J=7.7 Hz, 2H), 6.48 (s, 1H), 6.00 (s, 1H), 2.10 (s, 2H), 1.67-1.59 (m, 2H), 1.43 (s, 2H), 1.05 (s, 7H).

### Step 2: N-[4-(2,2-Dimethylcyclohexyl)-6-phenoxy-pyrimidin-2-yl]benzenesulfonamide

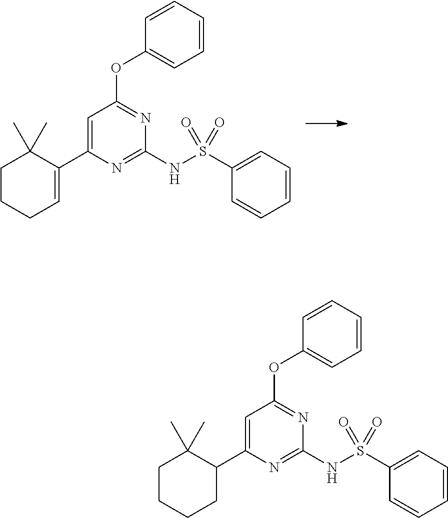 [SMILES: CC1(C)CCCC=C1c1cc(Oc2ccccc2)nc(NS(=O)(=O)c2ccccc2)n1 ; CC1(C)CCCCC1c1cc(Oc2ccccc2)nc(NS(=O)(=O)c2ccccc2)n1]

Pd/C (20 mg of 5% w/w, 0.009397 mmol) was added to N-[4-(6,6-dimethylcyclohexen-1-yl)-6-phenoxy-pyrimidin-2-yl]benzenesulfonamide (15 mg, 0.03444 mmol) in methanol (10 mL). The flask was purged with nitrogen and stirred under a balloon of hydrogen (approximately 0.06943 mg, ∞ L, 0.03444 mmol) at room temperature for 16 hours. The reaction mixture was filtered, concentrated under reduced pressure. The crude was suspended in DMSO, filtered and purified on reverse phase HPLC (HCl modifier, 30-99% ACN-H₂O) to give product N-[4-(2,2-dimethylcyclohexyl)-6-phenoxy-pyrimidin-2-yl]benzenesulfonamide (5.7 mg, 38%) as a white solid. ¹H NMR (400 MHz, DMSO) δ 11.53 (s, 1H), 7.50 (dd, J=80.7, 36.6 Hz, 8H), 7.20 (d, J=8.2 Hz, 2H), 6.38 (s, 1H), 2.36 (d, J=19.8 Hz, 1H), 1.73 (d, J=13.7 Hz, 2H), 1.42 (d, J=37.1 Hz, 3H), 1.22 (d, J=21.6 Hz, 3H), 0.73 (s, 6H). ESI-MS m/z calc. 437.1773, found 438.0 (M+1)⁺; Retention time: 2.05 minutes (LC method A).

### Example 337: Preparation of Compound 1130

### Step 1: N-[4-(2-methyl-1-methylene-propyl)-6-phenoxy-pyrimidin-2-yl]benzenesulfonamide

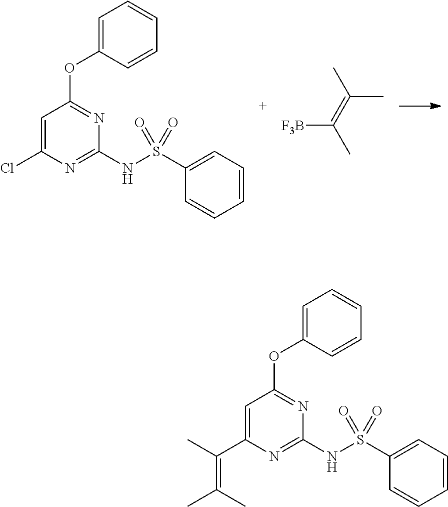

To a solution of N-(4-chloro-6-phenoxy-pyrimidin-2-yl)benzenesulfonamide (32 mg, 0.07827 mmol), 1,2-dimethylprop-1-enyl(trifluoro)boranuide (potassium salt) (22 mg, 0.1250 mmol) and (1,3-Bis(2,6-diisopropylphenyl)imidazolidene) (3-chloropyridyl) palladium(II)dichloride (10 mg, 0.01465 mmol) in EtOH (1 mL) was added K₂CO₃ (2 N in water) (approximately 156.6 μL of 2 M, 0.3131 mmol) and the reaction mixture was stirred at 120° C. for 20 minutes. At this time, more 1,2-dimethylprop-1-enyl(trifluoro)boranuide (Potassium Ion (1)) (22 mg, 0.1250 mmol), (1,3-Bis(2,6-diisopropylphenyl)imidazolidene) (3-chloropyridyl) palladium(II) dichloride (10 mg, 0.01465 mmol) and K₂CO₃ (2 N in water) (approximately 156.6 μL of 2 M, 0.3131 mmol) were added and the reaction mixture was stirred at 120° C. for 20 minutes. The reaction mixture was cooled and the EtOH was evaporated off. The material was then diluted with water and extracted with EtOAc (3×). Organics were combined, dried over Na₂SO₄ and evaporated. Purification by column chromatography (0-50% EtOAc in hexanes; 12 g silica) gave N-[4-(2-methyl-1-methylene-propyl)-6-phenoxy-pyrimidin-2-yl]benzenesulfonamide (9 mg, 29%) as a foam. ESI-MS m/z calc. 395.13037, found 396.2 (M+1)⁺; Retention time: 0.68 minutes; LC method D.

### Step 2: N-[4-(1,2-Dimethylpropyl)-6-phenoxy-pyrimidin-2-yl]benzenesulfonamide

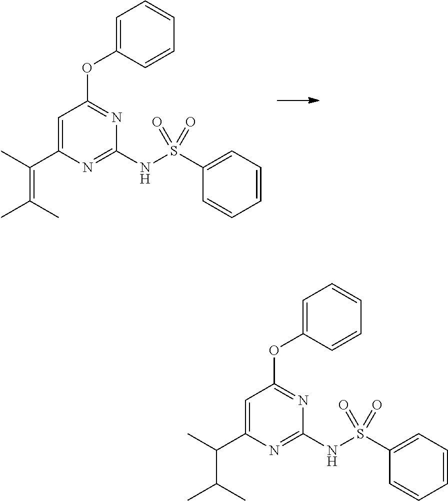 [SMILES: CC(C)=C(C)c1cc(Oc2ccccc2)nc(NS(=O)(=O)c2ccccc2)n1 ; CC(C)C(C)c1cc(Oc2ccccc2)nc(NS(=O)(=O)c2ccccc2)n1]

A suspension of N-[4-(2-methyl-1-methylene-propyl)-6-phenoxy-pyrimidin-2-yl]benzenesulfonamide (9 mg, 0.02276 mmol) and 10% Pd/C (3 mg, 0.02819 mmol) in MeOH (0.5 mL) was stirred under a balloon of hydrogen for 2 hours. The reaction mixture was filtered and purification by HPLC (1-99% ACN in water (HCl modifier)) gave N-[4-(1,2-dimethylpropyl)-6-phenoxy-pyrimidin-2-yl]benzenesulfonamide (2.8 mg, 31%) as a foam. ESI-MS m/z calc. 397.14603, found 398.3 (M+1)⁺; Retention time: 1.82 minutes; LC method A.

### Example 338: Preparation of Compound 1131

### Step 1: Ethyl 2-[2-(benzenesulfonamido)-6-phenoxy-pyrimidin-4-yl]-3-methyl-butanoate

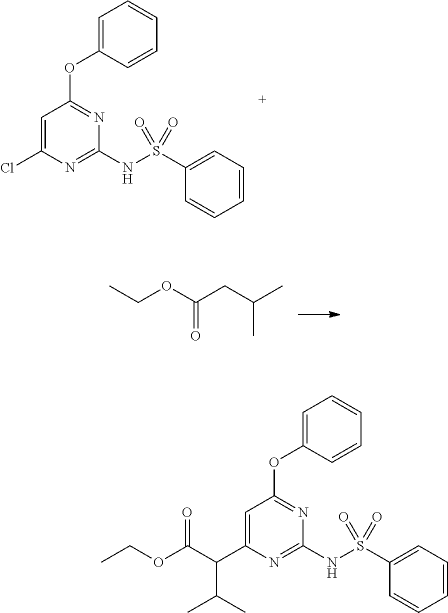

To a solution of ethyl 3-methylbutanoate (approximately 31.84 mg, 35.30 μL, 0.2446 mmol) in toluene (0.5 mL) at 0° C. was added NaHMDS (1 M in THF) (approximately 48.92 μL of 1 M, 0.04892 mmol) dropwise and the reaction mixture stirred while bubbling nitrogen through the solution for 5 minutes. At this time, allyl-[1,3-bis(2,6-diisopropylphenyl)imidazol-2-ylidene]-chloro-palladium (approximately 6.990 mg, 0.01223 mmol) and N-(4-chloro-6-phenoxy-pyrimidin-2-yl)benzenesulfonamide (20 mg, 0.04892 mmol) was added and the reaction mixture stirred at room temperature for 20 minutes then acetic acid (approximately 17.63 mg, 16.70 μL, 0.2935 mmol) was added. The reaction mixture was evaporated and diluted with MeOH, filtered and purification by HPLC (1-99% ACN in water (HCl modifier)) gave ethyl 2-[2-(benzenesulfonamido)-6-phenoxy-pyrimidin-4-yl]-3-methyl-butanoate (3 mg) as a foam. ESI-MS m/z calc. 455.1515, found 456.3 (M+1)⁺; Retention time: 1.89 minutes, LC method D.

### Example 339: Preparation of Compound 1132

### Step 1: N-[4-[1-(hydroxymethyl)-2-methyl-propyl]-6-phenoxy-pyrimidin-2-yl]benzenesulfonamide

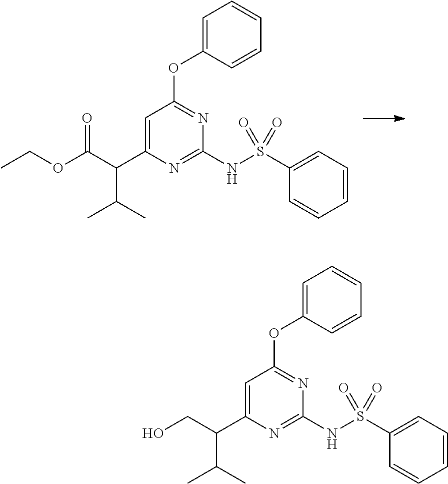 [SMILES: CCOC(=O)C(c1cc(Oc2ccccc2)nc(NS(=O)(=O)c2ccccc2)n1)C(C)C ; CC(C)C(CO)c1cc(Oc2ccccc2)nc(NS(=O)(=O)c2ccccc2)n1]

To a solution of ethyl 2-[2-(benzenesulfonamido)-6-phenoxy-pyrimidin-4-yl]-3 -methyl-butanoate (11 mg, 0.02415 mmol) in THE (0.5 mL) was added LiBH₄ (2M in THF) (approximately 24.15 μL of 2 M, 0.04830 mmol) and the reaction mixture stirred at rt for 1 hour, more LiBH₄ (2 M in THF) (approximately 24.15 μL of 2 M, 0.04830 mmol) was added and the reaction mixture stirred at room temperature for 30 minutes then more LiBH₄ (2 M in THF) (approximately 24.15 μL of 2 M, 0.04830 mmol) was added and the reaction mixture stirred at 40° C. for 20 minutes. The reaction mixture was then diluted with 0.25 mL MeOH and stirred for 5 minutes before quenching with acetic acid (approximately 36.26 mg, 34.34 μL, 0.6038 mmol). The reaction mixture was filtered and purification by HPLC (1-99% ACN in water (HCl modifier)) gave N-[4-[1-(hydroxymethyl)-2-methyl-propyl]-6-phenoxy-pyrimidin-2-yl]benzenesulfonamide (1.5 mg, 15%) as a foam. ESI-MS m/z calc. 413.14093, found 414.1 (M+1)⁺; Retention time: 1.51 minutes, LC method A.

### Example 340: Characterization of Compounds 1133-1274

The compounds in the following tables were prepared in a manner analogous to that described above using commercially available reagents and intermediates described herein.

| | | LCMS | | | |
|---|---|---|---|---|---|
| | | Room | | | |
| Compound | | temperature | Calc. | | LCMS |
| Number | Structure | (min) | mass | M + 1 | Method |
| 1133 | 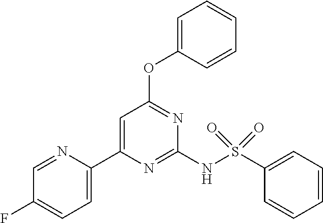 [SMILES: O=S(=O)(Nc1nc(Oc2ccccc2)cc(-c2ccc(F)cn2)n1)c1ccccc1] | 1.9 | 422.085 | 423.19 | A |
| 1134 | 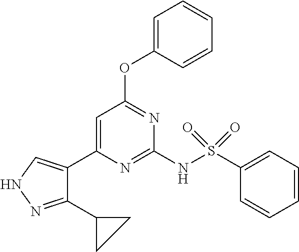 [SMILES: O=S(=O)(Nc1nc(Oc2ccccc2)cc(-c2c[nH]nc2C2CC2)n1)c1ccccc1] | 1.57 | 433.121 | 434.23 | A |
| 1135 | 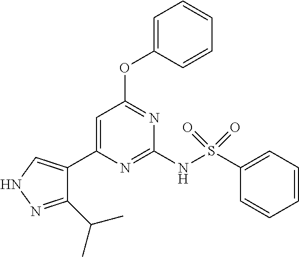 [SMILES: CC(C)c1n[nH]cc1-c1cc(Oc2ccccc2)nc(NS(=O)(=O)c2ccccc2)n1] | 1.64 | 435.137 | 436.24 | A |
| 1136 | 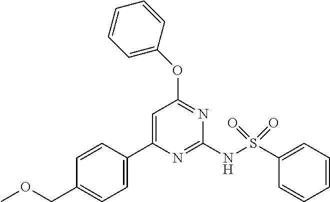 [SMILES: COCc1ccc(-c2cc(Oc3ccccc3)nc(NS(=O)(=O)c3ccccc3)n2)cc1] | 1.36 | 447.125 | 448.29 | R |
| 1137 | 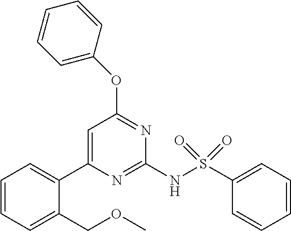 [SMILES: COCc1ccccc1-c1cc(Oc2ccccc2)nc(NS(=O)(=O)c2ccccc2)n1] | 1.33 | 447.125 | 448.29 | R |
| 1138 | 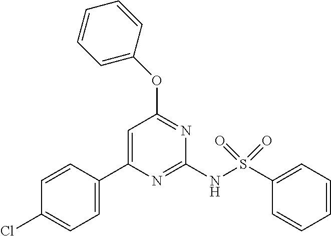 [SMILES: O=S(=O)(Nc1nc(Oc2ccccc2)cc(-c2ccc(Cl)cc2)n1)c1ccccc1] | 1.49 | 437.06 | 438.24 | R |
| 1139 | 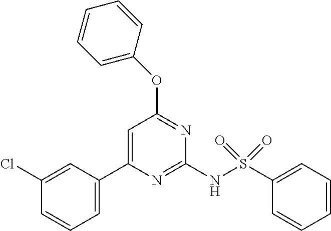 [SMILES: O=S(=O)(Nc1nc(Oc2ccccc2)cc(-c2cccc(Cl)c2)n1)c1ccccc1] | 1.48 | 437.06 | 438.24 | R |
| 1140 | 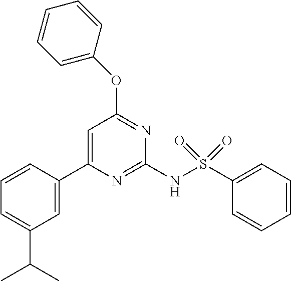 [SMILES: CC(C)c1cccc(-c2cc(Oc3ccccc3)nc(NS(=O)(=O)c3ccccc3)n2)c1] | 1.58 | 445.146 | 446.29 | R |
| 1141 | 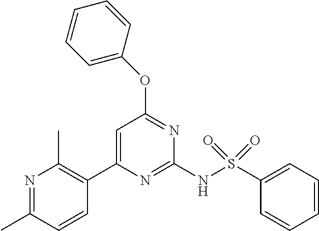 [SMILES: Cc1ccc(-c2cc(Oc3ccccc3)nc(NS(=O)(=O)c3ccccc3)n2)c(C)n1] | 1.19 | 432.126 | 433 | A |
| 1142 | 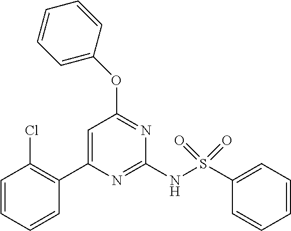 [SMILES: O=S(=O)(Nc1nc(Oc2ccccc2)cc(-c2ccccc2Cl)n1)c1ccccc1] | 1.96 | 437.06 | 437.9 | A |
| 1143 | 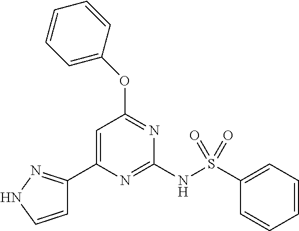 [SMILES: O=S(=O)(Nc1nc(Oc2ccccc2)cc(-c2cc[nH]n2)n1)c1ccccc1] | 1.06 | 393.09 | 394.23 | R |
| 1144 | 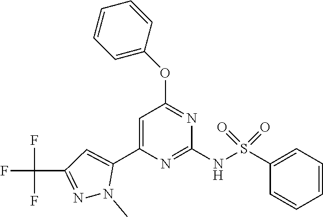 [SMILES: Cn1nc(C(F)(F)F)cc1-c1cc(Oc2ccccc2)nc(NS(=O)(=O)c2ccccc2)n1] | 1.99 | 475.093 | 476.48 | A |
| 1145 | 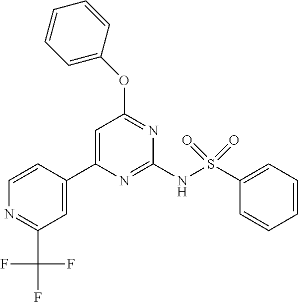 [SMILES: O=S(=O)(Nc1nc(Oc2ccccc2)cc(-c2ccnc(C(F)(F)F)c2)n1)c1ccccc1] | 1.91 | 472.082 | 473.47 | A |
| 1146 | 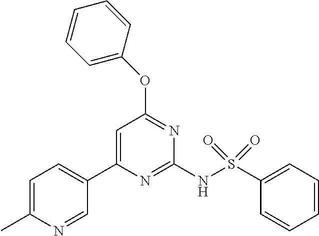 [SMILES: Cc1ccc(-c2cc(Oc3ccccc3)nc(NS(=O)(=O)c3ccccc3)n2)cn1] | 0.92 | 418.11 | 419.26 | R |
| 1147 | 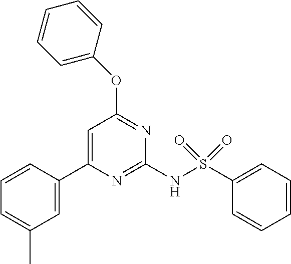 [SMILES: Cc1cccc(-c2cc(Oc3ccccc3)nc(NS(=O)(=O)c3ccccc3)n2)c1] | 1.46 | 417.115 | 418.28 | R |
| 1148 | 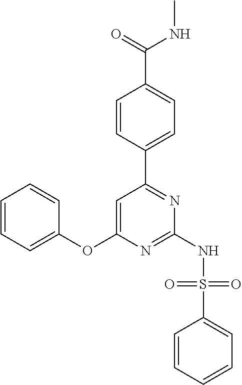 [SMILES: CNC(=O)c1ccc(-c2cc(Oc3ccccc3)nc(NS(=O)(=O)c3ccccc3)n2)cc1] | 1.72 | 460.121 | 461.28 | A |
| 1149 | 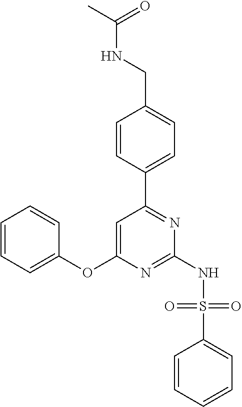 [SMILES: CC(=O)NCc1ccc(-c2cc(Oc3ccccc3)nc(NS(=O)(=O)c3ccccc3)n2)cc1] | 1.7 | 474.136 | 475.31 | A |
| 1150 | 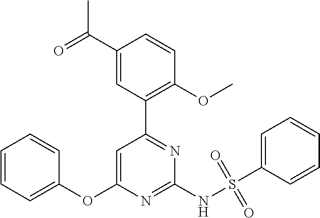 [SMILES: COc1ccc(C(C)=O)cc1-c1cc(Oc2ccccc2)nc(NS(=O)(=O)c2ccccc2)n1] | 1.93 | 475.12 | 476.29 | A |
| 1151 | 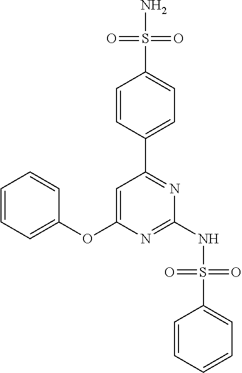 [SMILES: NS(=O)(=O)c1ccc(-c2cc(Oc3ccccc3)nc(NS(=O)(=O)c3ccccc3)n2)cc1] | 1.75 | 482.072 | 483.25 | A |
| 1152 | 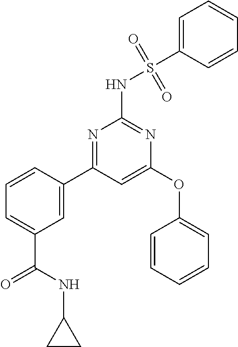 [SMILES: O=C(NC1CC1)c1cccc(-c2cc(Oc3ccccc3)nc(NS(=O)(=O)c3ccccc3)n2)c1] | 1.82 | 486.136 | 487.31 | A |
| 1153 | 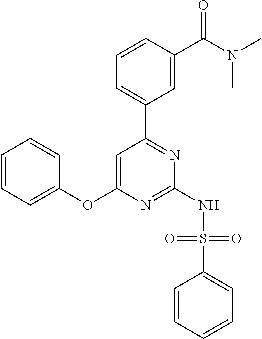 [SMILES: CN(C)C(=O)c1cccc(-c2cc(Oc3ccccc3)nc(NS(=O)(=O)c3ccccc3)n2)c1] | 1.78 | 474.136 | 475.31 | A |
| 1154 | 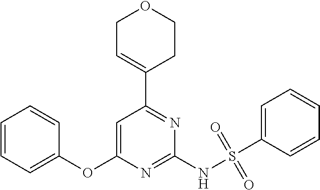 [SMILES: O=S(=O)(Nc1nc(Oc2ccccc2)cc(C2=CCOCC2)n1)c1ccccc1] | 1.78 | 409.11 | 410.25 | A |
| 1155 | 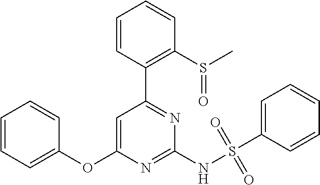 [SMILES: CS(=O)c1ccccc1-c1cc(Oc2ccccc2)nc(NS(=O)(=O)c2ccccc2)n1] | 1.77 | 465.082 | 466.28 | A |
| 1156 | 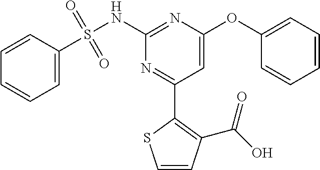 [SMILES: O=C(O)c1ccsc1-c1cc(Oc2ccccc2)nc(NS(=O)(=O)c2ccccc2)n1] | 1.77 | 453.045 | 454.21 | A |
| 1157 | 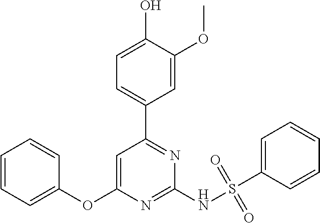 [SMILES: COc1cc(-c2cc(Oc3ccccc3)nc(NS(=O)(=O)c3ccccc3)n2)ccc1O] | 1.82 | 449.105 | 450.29 | A |
| 1158 | 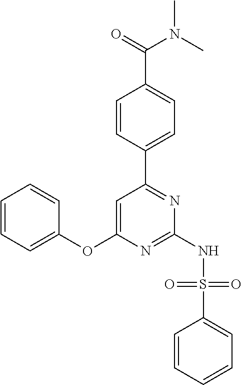 [SMILES: CN(C)C(=O)c1ccc(-c2cc(Oc3ccccc3)nc(NS(=O)(=O)c3ccccc3)n2)cc1] | 1.77 | 474.136 | 475.34 | A |
| 1159 | 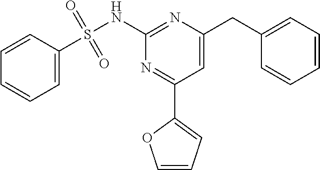 [SMILES: O=S(=O)(Nc1nc(Cc2ccccc2)cc(-c2ccco2)n1)c1ccccc1] | 1.93 | 393.078 | 394.25 | A |
| 1160 | 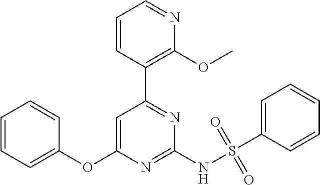 [SMILES: COc1ncccc1-c1cc(Oc2ccccc2)nc(NS(=O)(=O)c2ccccc2)n1] | 1.97 | 434.105 | 435.27 | A |
| 1161 | 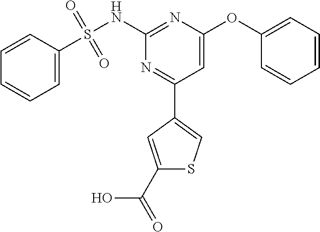 [SMILES: O=C(O)c1cc(-c2cc(Oc3ccccc3)nc(NS(=O)(=O)c3ccccc3)n2)cs1] | 1.78 | 453.045 | 454.17 | A |
| 1162 | 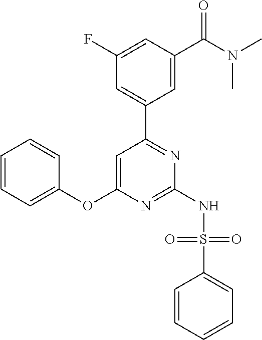 [SMILES: CN(C)C(=O)c1cc(F)cc(-c2cc(Oc3ccccc3)nc(NS(=O)(=O)c3ccccc3)n2)c1] | 1.85 | 492.127 | 493.33 | A |
| 1163 | 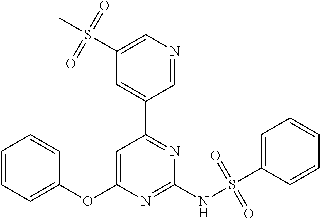 [SMILES: CS(=O)(=O)c1cncc(-c2cc(Oc3ccccc3)nc(NS(=O)(=O)c3ccccc3)n2)c1] | 1.72 | 482.072 | 483.25 | A |
| 1164 | 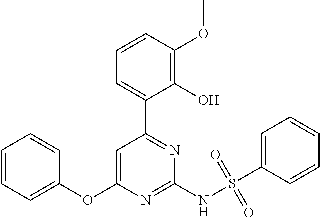 [SMILES: COc1cccc(-c2cc(Oc3ccccc3)nc(NS(=O)(=O)c3ccccc3)n2)c1O] | 2.02 | 449.105 | 450.29 | A |
| 1165 | 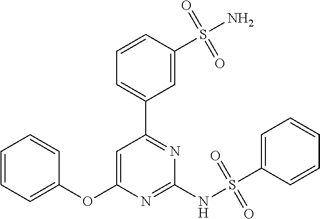 [SMILES: NS(=O)(=O)c1cccc(-c2cc(Oc3ccccc3)nc(NS(=O)(=O)c3ccccc3)n2)c1] | 1.75 | 482.072 | 483.25 | A |
| 1166 | 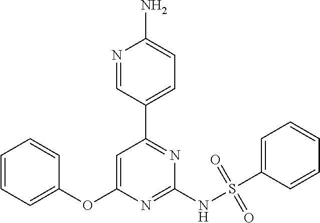 [SMILES: Nc1ccc(-c2cc(Oc3ccccc3)nc(NS(=O)(=O)c3ccccc3)n2)cn1] | 1.43 | 419.105 | 420.26 | A |
| 1167 | 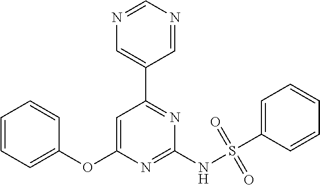 [SMILES: O=S(=O)(Nc1nc(Oc2ccccc2)cc(-c2cncnc2)n1)c1ccccc1] | 1.82 | 405.09 | 406.35 | A |
| 1168 | 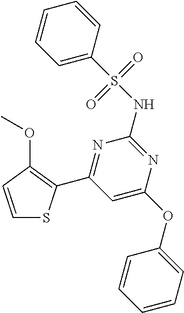 [SMILES: COc1ccsc1-c1cc(Oc2ccccc2)nc(NS(=O)(=O)c2ccccc2)n1] | 1.82 | 439.066 | 440.12 | A |
| 1169 | 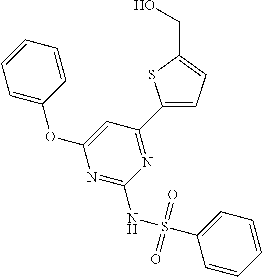 [SMILES: O=S(=O)(Nc1nc(Oc2ccccc2)cc(-c2ccc(CO)s2)n1)c1ccccc1] | 1.6 | 439.066 | 440.12 | A |
| 1170 | 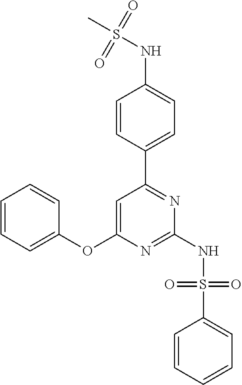 [SMILES: CS(=O)(=O)Nc1ccc(-c2cc(Oc3ccccc3)nc(NS(=O)(=O)c3ccccc3)n2)cc1] | 1.64 | 496.088 | 497.2 | A |
| 1171 | 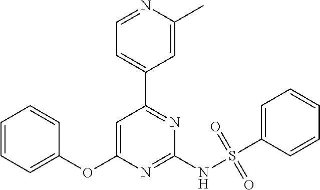 [SMILES: Cc1cc(-c2cc(Oc3ccccc3)nc(NS(=O)(=O)c3ccccc3)n2)ccn1] | 1.3 | 418.11 | 419.14 | A |
| 1172 | 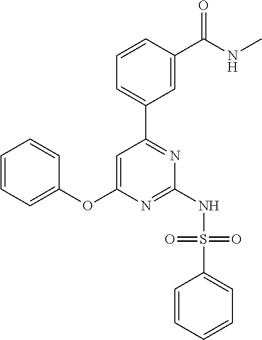 [SMILES: CNC(=O)c1cccc(-c2cc(Oc3ccccc3)nc(NS(=O)(=O)c3ccccc3)n2)c1] | 1.57 | 460.121 | 461.23 | A |
| 1173 | 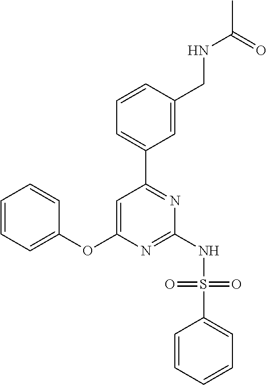 [SMILES: CC(=O)NCc1cccc(-c2cc(Oc3ccccc3)nc(NS(=O)(=O)c3ccccc3)n2)c1] | 1.56 | 474.136 | 475.22 | A |
| 1174 | 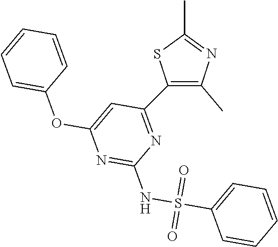 [SMILES: Cc1nc(C)c(-c2cc(Oc3ccccc3)nc(NS(=O)(=O)c3ccccc3)n2)s1] | 1.69 | 438.082 | 439.37 | A |
| 1175 | 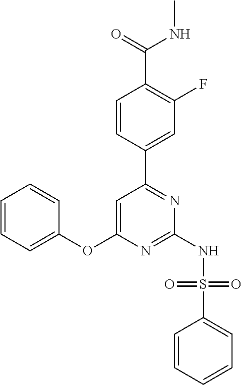 [SMILES: CNC(=O)c1ccc(-c2cc(Oc3ccccc3)nc(NS(=O)(=O)c3ccccc3)n2)cc1F] | 1.61 | 478.111 | 479.19 | A |
| 1176 | 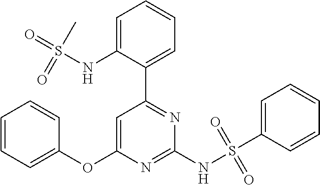 [SMILES: CS(=O)(=O)Nc1ccccc1-c1cc(Oc2ccccc2)nc(NS(=O)(=O)c2ccccc2)n1] | 1.77 | 496.088 | 497.2 | A |
| 1177 | 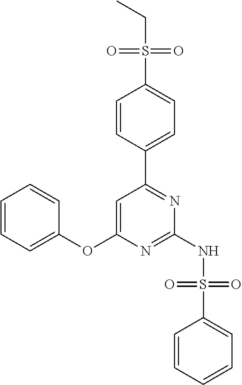 [SMILES: CCS(=O)(=O)c1ccc(-c2cc(Oc3ccccc3)nc(NS(=O)(=O)c3ccccc3)n2)cc1] | 1.73 | 495.092 | 496.2 | A |
| 1178 | 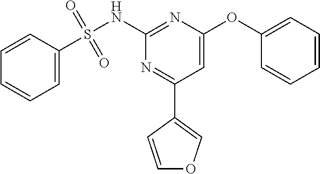 [SMILES: O=S(=O)(Nc1nc(Oc2ccccc2)cc(-c2ccoc2)n1)c1ccccc1] | 1.69 | 393.078 | 394.13 | A |
| 1179 | 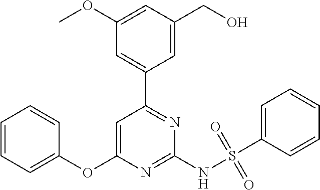 [SMILES: COc1cc(CO)cc(-c2cc(Oc3ccccc3)nc(NS(=O)(=O)c3ccccc3)n2)c1] | 1.94 | 463.12 | 464.09 | A |
| 1180 | 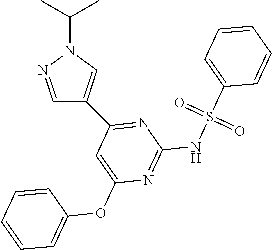 [SMILES: CC(C)n1cc(-c2cc(Oc3ccccc3)nc(NS(=O)(=O)c3ccccc3)n2)cn1] | 1.67 | 435.137 | 436.15 | A |
| 1181 | 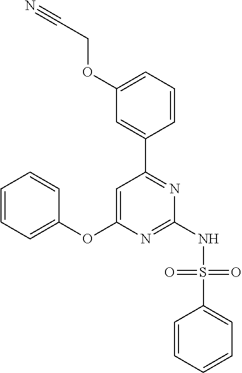 [SMILES: N#CCOc1cccc(-c2cc(Oc3ccccc3)nc(NS(=O)(=O)c3ccccc3)n2)c1] | 1.42 | 458.105 | 459.15 | A |
| 1182 | 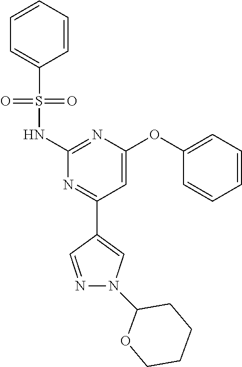 [SMILES: O=S(=O)(Nc1nc(Oc2ccccc2)cc(-c2cnn(C3CCCCO3)c2)n1)c1ccccc1] | 1.7 | 477.147 | 478.17 | A |
| 1183 | 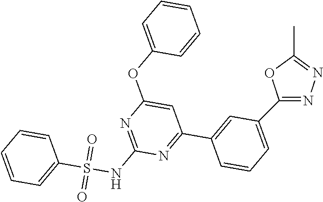 [SMILES: Cc1nnc(-c2cccc(-c3cc(Oc4ccccc4)nc(NS(=O)(=O)c4ccccc4)n3)c2)o1] | 1.75 | 485.116 | 486.1 | A |
| 1184 | 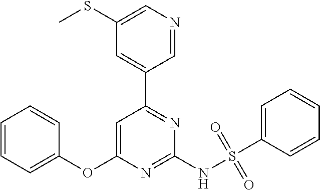 [SMILES: CSc1cncc(-c2cc(Oc3ccccc3)nc(NS(=O)(=O)c3ccccc3)n2)c1] | 1.66 | 450.082 | 451.08 | A |
| 1185 | 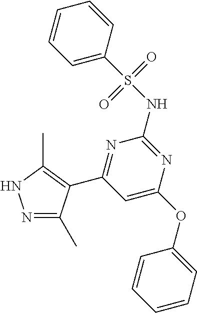 [SMILES: Cc1n[nH]c(C)c1-c1cc(Oc2ccccc2)nc(NS(=O)(=O)c2ccccc2)n1] | 1.9 | 421.121 | 420.09 | A |
| 1186 | 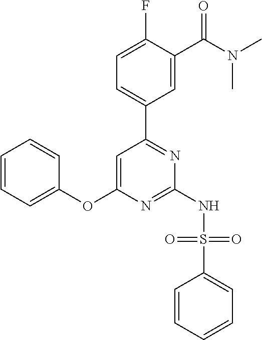 [SMILES: CN(C)C(=O)c1cc(-c2cc(Oc3ccccc3)nc(NS(=O)(=O)c3ccccc3)n2)ccc1F] | 1.68 | 492.127 | 493.1 | A |
| 1187 | 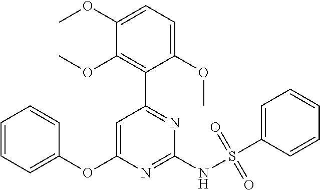 [SMILES: COc1ccc(OC)c(-c2cc(Oc3ccccc3)nc(NS(=O)(=O)c3ccccc3)n2)c1OC] | 1.73 | 493.131 | 494.16 | A |
| 1188 | 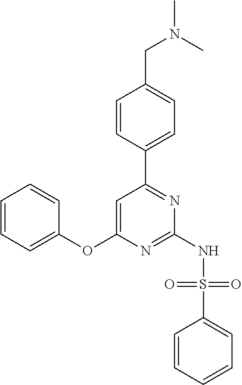 [SMILES: CN(C)Cc1ccc(-c2cc(Oc3ccccc3)nc(NS(=O)(=O)c3ccccc3)n2)cc1] | 1.36 | 460.157 | 461.12 | A |
| 1190 | 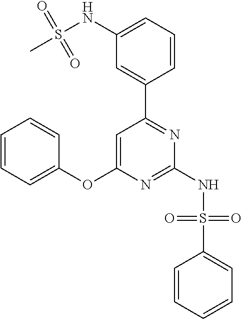 [SMILES: CS(=O)(=O)Nc1cccc(-c2cc(Oc3ccccc3)nc(NS(=O)(=O)c3ccccc3)n2)c1] | 1.69 | 496.088 | 497.07 | A |
| 1191 | 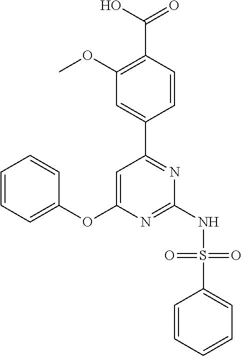 [SMILES: COc1cc(-c2cc(Oc3ccccc3)nc(NS(=O)(=O)c3ccccc3)n2)ccc1C(=O)O] | 1.65 | 477.099 | 478.1 | A |
| 1192 | 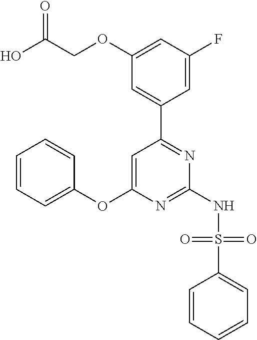 [SMILES: O=C(O)COc1cc(F)cc(-c2cc(Oc3ccccc3)nc(NS(=O)(=O)c3ccccc3)n2)c1] | 1.74 | 495.09 | 496.08 | A |
| 1193 | 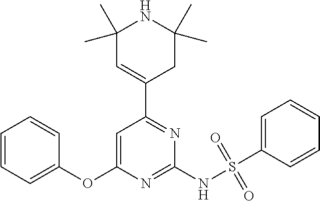 [SMILES: CC1(C)C=C(c2cc(Oc3ccccc3)nc(NS(=O)(=O)c3ccccc3)n2)CC(C)(C)N1] | 1.34 | 464.188 | 465.22 | A |
| 1194 | 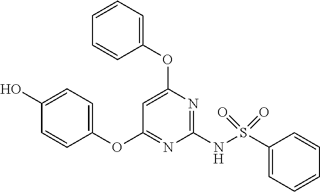 [SMILES: O=S(=O)(Nc1nc(Oc2ccccc2)cc(Oc2ccc(O)cc2)n1)c1ccccc1] | 1.65 | 419.094 | 420.05 | A |
| 1195 | 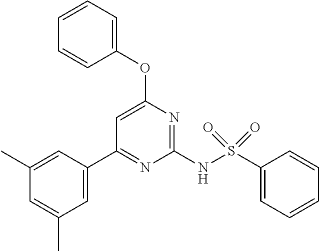 [SMILES: Cc1cc(C)cc(-c2cc(Oc3ccccc3)nc(NS(=O)(=O)c3ccccc3)n2)c1] | 2.09 | 431.13 | 432.48 | A |
| 1196 | 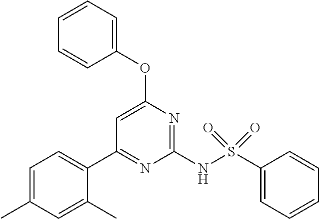 [SMILES: Cc1ccc(-c2cc(Oc3ccccc3)nc(NS(=O)(=O)c3ccccc3)n2)c(C)c1] | 2 | 431.13 | 432.48 | A |
| 1197 | 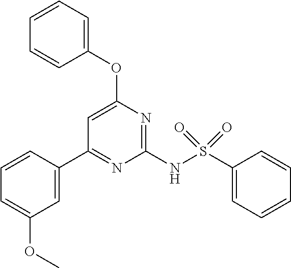 [SMILES: COc1cccc(-c2cc(Oc3ccccc3)nc(NS(=O)(=O)c3ccccc3)n2)c1] | 1.82 | 433.11 | 434.29 | A |
| 1198 | 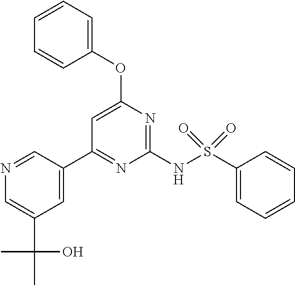 [SMILES: CC(C)(O)c1cncc(-c2cc(Oc3ccccc3)nc(NS(=O)(=O)c3ccccc3)n2)c1] | 1.15 | 462.136 | 463.31 | A |
| 1199 | 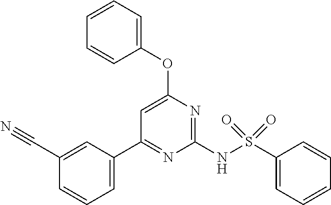 [SMILES: N#Cc1cccc(-c2cc(Oc3ccccc3)nc(NS(=O)(=O)c3ccccc3)n2)c1] | 1.8 | 428.094 | 429.28 | A |
| 1200 | 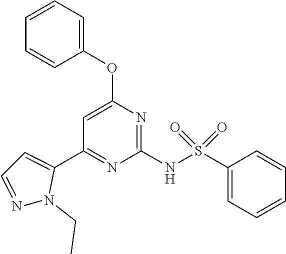 [SMILES: CCn1nccc1-c1cc(Oc2ccccc2)nc(NS(=O)(=O)c2ccccc2)n1] | 1.71 | 421.121 | 422.12 | A |
| 1201 | 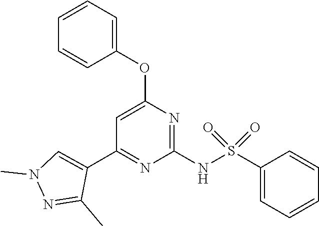 [SMILES: Cc1nn(C)cc1-c1cc(Oc2ccccc2)nc(NS(=O)(=O)c2ccccc2)n1] | 1.58 | 421.121 | 422.14 | A |
| 1202 | 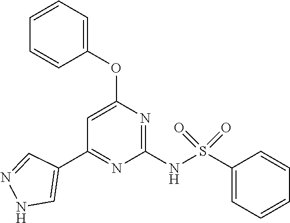 [SMILES: O=S(=O)(Nc1nc(Oc2ccccc2)cc(-c2cn[nH]c2)n1)c1ccccc1] | 1.71 | 393.09 | 392.39 | A |
| 1203 | 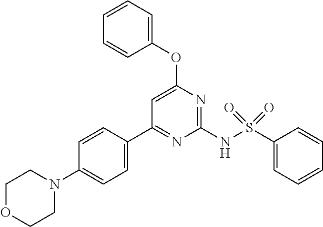 [SMILES: O=S(=O)(Nc1nc(Oc2ccccc2)cc(-c2ccc(N3CCOCC3)cc2)n1)c1ccccc1] | 1.81 | 488.152 | 489.33 | A |
| 1204 | 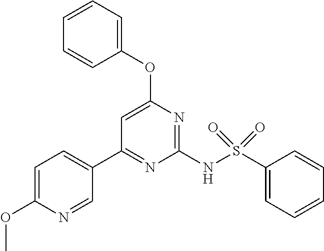 [SMILES: COc1ccc(-c2cc(Oc3ccccc3)nc(NS(=O)(=O)c3ccccc3)n2)cn1] | 1.76 | 434.105 | 435.15 | A |
| 1205 | 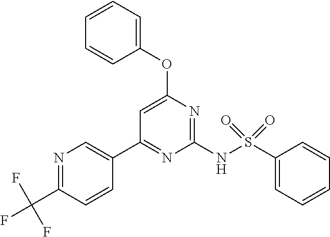 [SMILES: O=S(=O)(Nc1nc(Oc2ccccc2)cc(-c2ccc(C(F)(F)F)nc2)n1)c1ccccc1] | 1.83 | 472.082 | 473.12 | A |
| 1206 | 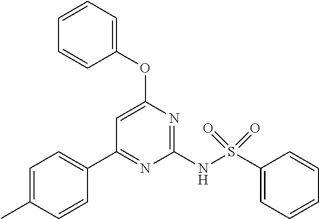 [SMILES: Cc1ccc(-c2cc(Oc3ccccc3)nc(NS(=O)(=O)c3ccccc3)n2)cc1] | 1.98 | 417.115 | 418.48 | A |
| 1207 | 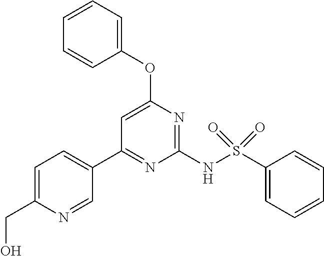 [SMILES: O=S(=O)(Nc1nc(Oc2ccccc2)cc(-c2ccc(CO)nc2)n1)c1ccccc1] | 1.35 | 434.105 | 435.15 | A |
| 1208 | 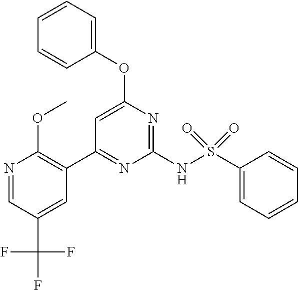 [SMILES: COc1ncc(C(F)(F)F)cc1-c1cc(Oc2ccccc2)nc(NS(=O)(=O)c2ccccc2)n1] | 2 | 502.092 | 503.01 | A |
| 1209 | 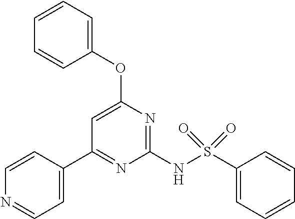 [SMILES: O=S(=O)(Nc1nc(Oc2ccccc2)cc(-c2ccncc2)n1)c1ccccc1] | 1.34 | 404.094 | 405.26 | A |
| 1210 | 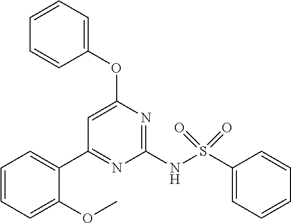 [SMILES: COc1ccccc1-c1cc(Oc2ccccc2)nc(NS(=O)(=O)c2ccccc2)n1] | 1.84 | 433.11 | 434.26 | A |
| 1211 | 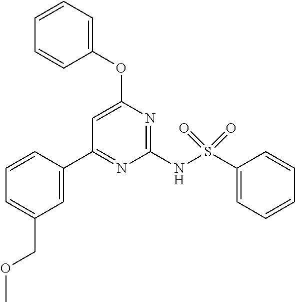 [SMILES: COCc1cccc(-c2cc(Oc3ccccc3)nc(NS(=O)(=O)c3ccccc3)n2)c1] | 1.82 | 447.125 | 448.16 | A |
| 1212 | 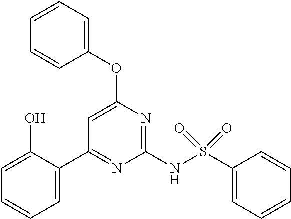 [SMILES: O=S(=O)(Nc1nc(Oc2ccccc2)cc(-c2ccccc2O)n1)c1ccccc1] | 1.905 | 419.094 | 420.25 | A |
| 1213 | 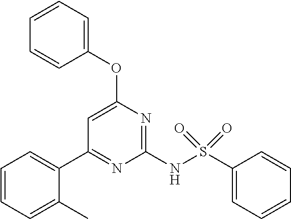 [SMILES: Cc1ccccc1-c1cc(Oc2ccccc2)nc(NS(=O)(=O)c2ccccc2)n1] | 1.89 | 417.115 | 418.48 | A |
| 1214 | 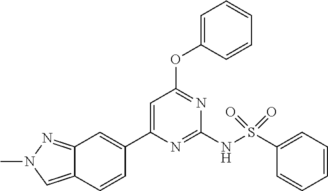 [SMILES: Cn1cc2ccc(-c3cc(Oc4ccccc4)nc(NS(=O)(=O)c4ccccc4)n3)cc2n1] | 1.63 | 457.121 | 458.08 | A |
| 1215 | 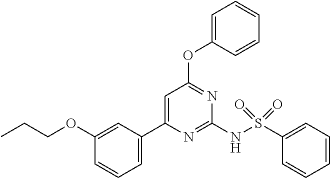 [SMILES: CCCOc1cccc(-c2cc(Oc3ccccc3)nc(NS(=O)(=O)c3ccccc3)n2)c1] | 2.06 | 461.141 | 462.11 | A |
| 1216 | 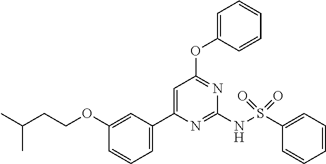 [SMILES: CC(C)CCOc1cccc(-c2cc(Oc3ccccc3)nc(NS(=O)(=O)c3ccccc3)n2)c1] | 2.24 | 489.172 | 490.13 | A |
| 1217 | 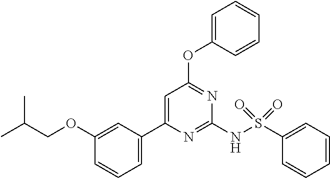 [SMILES: CC(C)COc1cccc(-c2cc(Oc3ccccc3)nc(NS(=O)(=O)c3ccccc3)n2)c1] | 2.17 | 475.157 | 476.12 | A |
| 1218 | 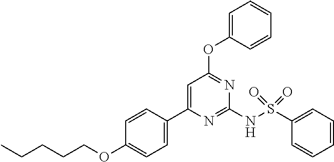 [SMILES: CCCCCOc1ccc(-c2cc(Oc3ccccc3)nc(NS(=O)(=O)c3ccccc3)n2)cc1] | 2.21 | 489.172 | 490.13 | A |
| 1219 | 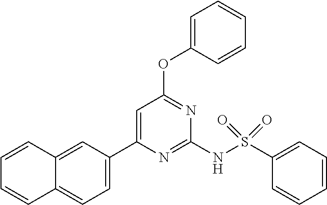 [SMILES: O=S(=O)(Nc1nc(Oc2ccccc2)cc(-c2ccc3ccccc3c2)n1)c1ccccc1] | 2.02 | 453.115 | 454.05 | A |
| 1220 | 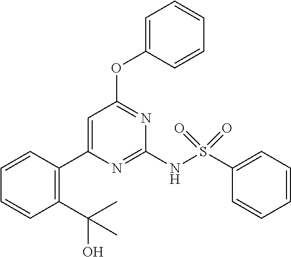 [SMILES: CC(C)(O)c1ccccc1-c1cc(Oc2ccccc2)nc(NS(=O)(=O)c2ccccc2)n1] | 1.75 | 461.141 | 462.2 | A |
| 1221 | 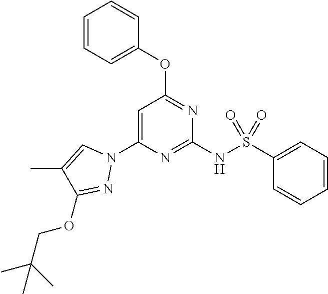 [SMILES: Cc1cn(-c2cc(Oc3ccccc3)nc(NS(=O)(=O)c3ccccc3)n2)nc1OCC(C)(C)C] | 2.43 | 493.178 | 494.4 | A |
| 1222 | 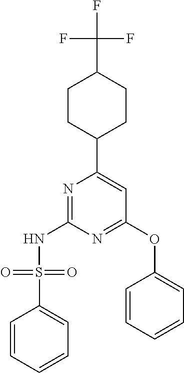 [SMILES: O=S(=O)(Nc1nc(Oc2ccccc2)cc(C2CCC(C(F)(F)F)CC2)n1)c1ccccc1] | 1.98 | 477.133 | 478 | A |
| 1223 | 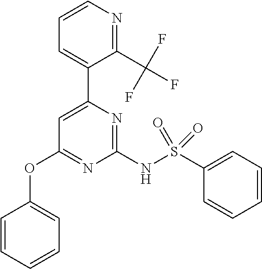 [SMILES: O=S(=O)(Nc1nc(Oc2ccccc2)cc(-c2cccnc2C(F)(F)F)n1)c1ccccc1] | 1.73 | 472.082 | 473 | A |
| 1224 | 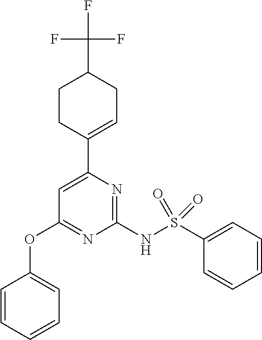 [SMILES: O=S(=O)(Nc1nc(Oc2ccccc2)cc(C2=CCC(C(F)(F)F)CC2)n1)c1ccccc1] | 2.04 | 475.118 | 475 | A |
| 1225 | 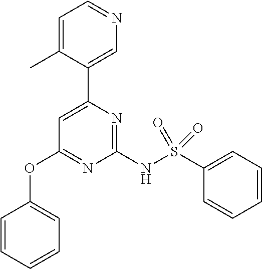 [SMILES: Cc1ccncc1-c1cc(Oc2ccccc2)nc(NS(=O)(=O)c2ccccc2)n1] | 1.17 | 418.11 | 419 | A |
| 1226 | 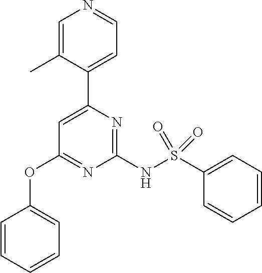 [SMILES: Cc1cnccc1-c1cc(Oc2ccccc2)nc(NS(=O)(=O)c2ccccc2)n1] | 1.18 | 418.11 | 419 | A |
| 1227 | 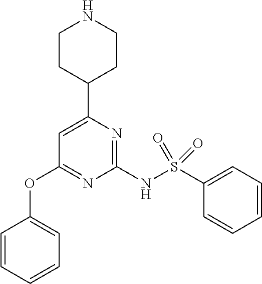 [SMILES: O=S(=O)(Nc1nc(Oc2ccccc2)cc(C2CCNCC2)n1)c1ccccc1] | 1.09 | 410.141 | 411 | A |
| 1228 | 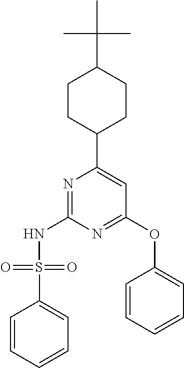 [SMILES: CC(C)(C)C1CCC(c2cc(Oc3ccccc3)nc(NS(=O)(=O)c3ccccc3)n2)CC1] | 2.35 | 465.209 | 466 | A |
| 1229 | 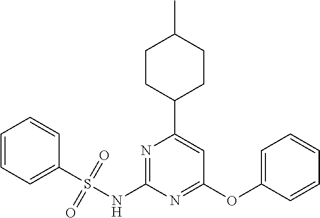 [SMILES: CC1CCC(c2cc(Oc3ccccc3)nc(NS(=O)(=O)c3ccccc3)n2)CC1] | 2.05 | 423.162 | 424 | A |
| 1230 | 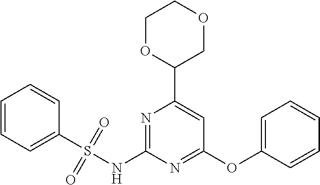 [SMILES: O=S(=O)(Nc1nc(Oc2ccccc2)cc(C2COCCO2)n1)c1ccccc1] | 1.57 | 413.105 | 414 | A |
| 1231 | 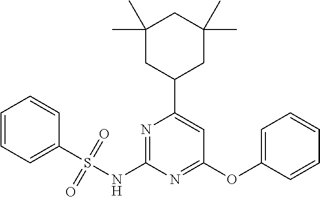 [SMILES: CC1(C)CC(c2cc(Oc3ccccc3)nc(NS(=O)(=O)c3ccccc3)n2)CC(C)(C)C1] | 2.31 | 465.209 | 466 | A |
| 1232 | 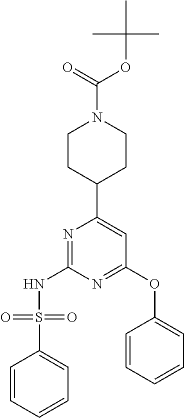 [SMILES: CC(C)(C)OC(=O)N1CCC(c2cc(Oc3ccccc3)nc(NS(=O)(=O)c3ccccc3)n2)CC1] | 1.92 | 510.194 | 511 | A |
| 1233 | 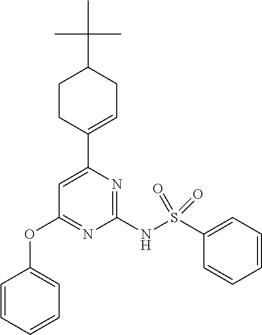 [SMILES: CC(C)(C)C1CC=C(c2cc(Oc3ccccc3)nc(NS(=O)(=O)c3ccccc3)n2)CC1] | 2.38 | 463.193 | 464 | A |
| 1234 | 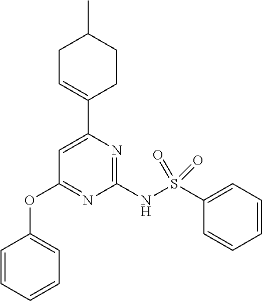 [SMILES: CC1CC=C(c2cc(Oc3ccccc3)nc(NS(=O)(=O)c3ccccc3)n2)CC1] | 2.11 | 421.146 | 422 | A |
| 1235 | 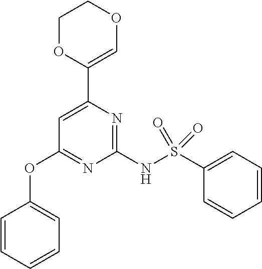 [SMILES: O=S(=O)(Nc1nc(Oc2ccccc2)cc(C2=COCCO2)n1)c1ccccc1] | 1.7 | 411.089 | 412 | A |
| 1236 | 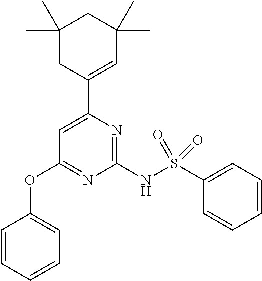 [SMILES: CC1(C)C=C(c2cc(Oc3ccccc3)nc(NS(=O)(=O)c3ccccc3)n2)CC(C)(C)C1] | 2.35 | 463.193 | 464 | A |
| 1237 | 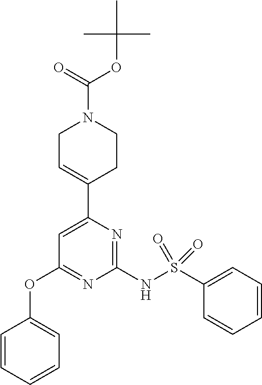 [SMILES: CC(C)(C)OC(=O)N1CC=C(c2cc(Oc3ccccc3)nc(NS(=O)(=O)c3ccccc3)n2)CC1] | 2 | 508.178 | 509 | A |
| 1238 | 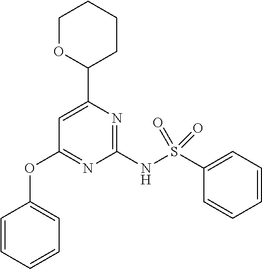 [SMILES: O=S(=O)(Nc1nc(Oc2ccccc2)cc(C2CCCCO2)n1)c1ccccc1] | 1.73 | 411.125 | 412 | A |
| 1239 | 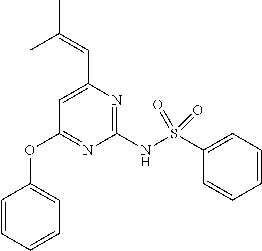 [SMILES: CC(C)=Cc1cc(Oc2ccccc2)nc(NS(=O)(=O)c2ccccc2)n1] | 1.7 | 381.115 | 382 | A |
| 1240 | 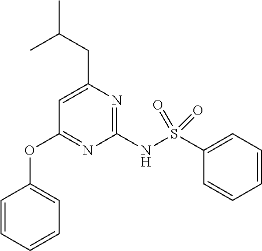 [SMILES: CC(C)Cc1cc(Oc2ccccc2)nc(NS(=O)(=O)c2ccccc2)n1] | 1.68 | 383.13 | 384 | A |
| 1241 | 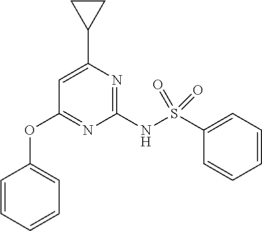 [SMILES: O=S(=O)(Nc1nc(Oc2ccccc2)cc(C2CC2)n1)c1ccccc1] | 1.63 | 367.099 | 368 | A |
| 1242 | 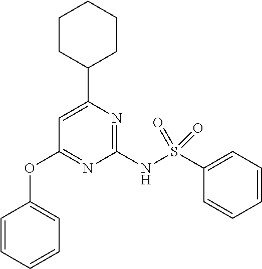 [SMILES: O=S(=O)(Nc1nc(Oc2ccccc2)cc(C2CCCCC2)n1)c1ccccc1] | 1.915 | 409.146 | 410 | A |
| 1243 | 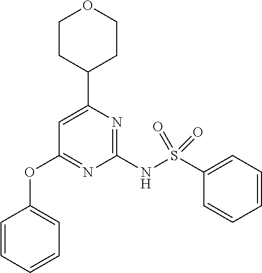 [SMILES: O=S(=O)(Nc1nc(Oc2ccccc2)cc(C2CCOCC2)n1)c1ccccc1] | 1.48 | 411.125 | 412 | A |
| 1244 | 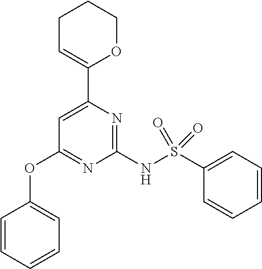 [SMILES: O=S(=O)(Nc1nc(Oc2ccccc2)cc(C2=CCCCO2)n1)c1ccccc1] | 1.82 | 409.11 | 410 | A |
| 1245 | 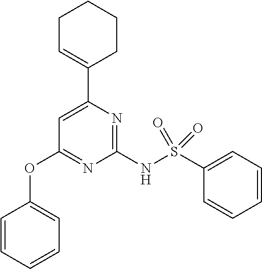 [SMILES: O=S(=O)(Nc1nc(Oc2ccccc2)cc(C2=CCCCC2)n1)c1ccccc1] | 1.97 | 407.13 | 408 | A |
| 1246 | 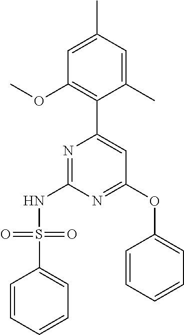 [SMILES: COc1cc(C)cc(C)c1-c1cc(Oc2ccccc2)nc(NS(=O)(=O)c2ccccc2)n1] | 1.9 | 461.141 | 462 | A |
| 1247 | 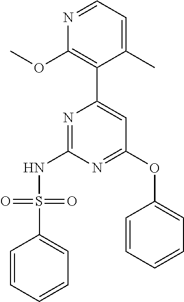 [SMILES: COc1nccc(C)c1-c1cc(Oc2ccccc2)nc(NS(=O)(=O)c2ccccc2)n1] | 1.7 | 448.121 | 449 | A |
| 1248 | 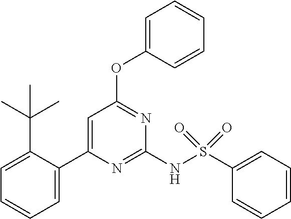 [SMILES: CC(C)(C)c1ccccc1-c1cc(Oc2ccccc2)nc(NS(=O)(=O)c2ccccc2)n1] | 2.09 | 459.162 | 460 | A |
| 1249 | 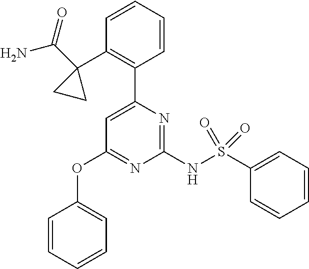 [SMILES: NC(=O)C1(c2ccccc2-c2cc(Oc3ccccc3)nc(NS(=O)(=O)c3ccccc3)n2)CC1] | 1.59 | 486.136 | 487 | A |
| 1250 | 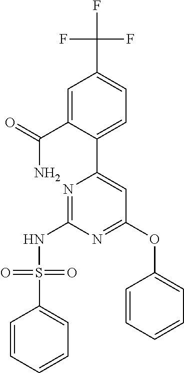 [SMILES: NC(=O)c1cc(C(F)(F)F)ccc1-c1cc(Oc2ccccc2)nc(NS(=O)(=O)c2ccccc2)n1] | 1.64 | 514.092 | 515 | A |
| 1251 | 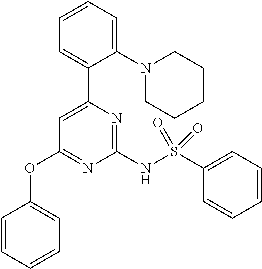 [SMILES: O=S(=O)(Nc1nc(Oc2ccccc2)cc(-c2ccccc2N2CCCCC2)n1)c1ccccc1] | 1.48 | 486.173 | 487 | A |
| 1252 | 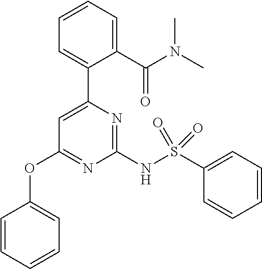 [SMILES: CN(C)C(=O)c1ccccc1-c1cc(Oc2ccccc2)nc(NS(=O)(=O)c2ccccc2)n1] | 1.6 | 474.136 | 475 | A |
| 1253 | 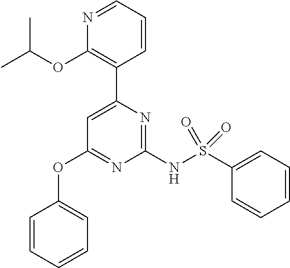 [SMILES: CC(C)Oc1ncccc1-c1cc(Oc2ccccc2)nc(NS(=O)(=O)c2ccccc2)n1] | 2.03 | 462.136 | 463 | A |
| 1254 | 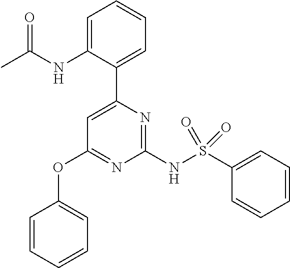 [SMILES: CC(=O)Nc1ccccc1-c1cc(Oc2ccccc2)nc(NS(=O)(=O)c2ccccc2)n1] | 1.69 | 460.121 | 461 | A |
| 1255 | 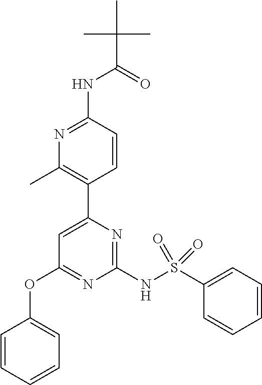 [SMILES: Cc1nc(NC(=O)C(C)(C)C)ccc1-c1cc(Oc2ccccc2)nc(NS(=O)(=O)c2ccccc2)n1] | 1.78 | 517.178 | 518 | A |
| 1256 | 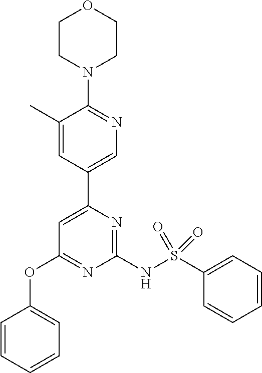 [SMILES: Cc1cc(-c2cc(Oc3ccccc3)nc(NS(=O)(=O)c3ccccc3)n2)cnc1N1CCOCC1] | 1.53 | 503.163 | 504 | A |
| 1257 | 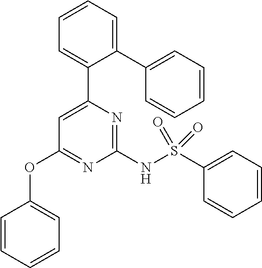 [SMILES: O=S(=O)(Nc1nc(Oc2ccccc2)cc(-c2ccccc2-c2ccccc2)n1)c1ccccc1] | 2.04 | 479.13 | 480 | A |
| 1258 | 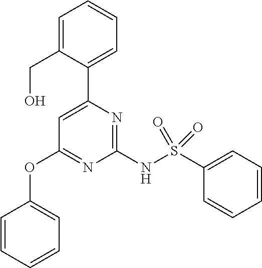 [SMILES: O=S(=O)(Nc1nc(Oc2ccccc2)cc(-c2ccccc2CO)n1)c1ccccc1] | 1.62 | 433.11 | 434 | A |
| 1259 | 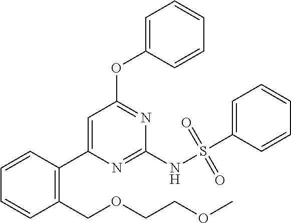 [SMILES: COCCOCc1ccccc1-c1cc(Oc2ccccc2)nc(NS(=O)(=O)c2ccccc2)n1] | 1.78 | 491.151 | 492 | A |
| 1260 | 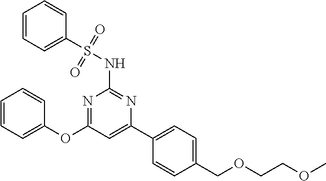 [SMILES: COCCOCc1ccc(-c2cc(Oc3ccccc3)nc(NS(=O)(=O)c3ccccc3)n2)cc1] | 1.82 | 491.151 | 492 | A |
| 1261 | 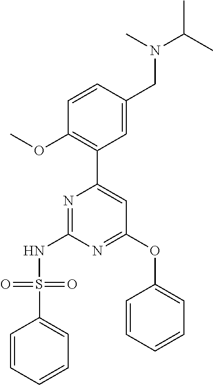 [SMILES: COc1ccc(CN(C)C(C)C)cc1-c1cc(Oc2ccccc2)nc(NS(=O)(=O)c2ccccc2)n1] | 1.33 | 518.199 | 519 | A |
| 1262 | 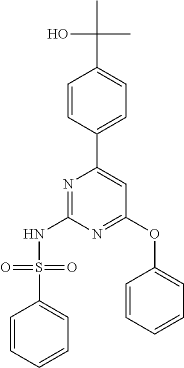 [SMILES: CC(C)(O)c1ccc(-c2cc(Oc3ccccc3)nc(NS(=O)(=O)c3ccccc3)n2)cc1] | 1.71 | 461.141 | 462 | A |
| 1263 | 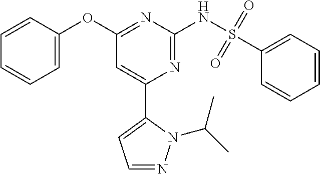 [SMILES: CC(C)n1nccc1-c1cc(Oc2ccccc2)nc(NS(=O)(=O)c2ccccc2)n1] | 1.8 | 435.137 | 436 | A |
| 1264 | 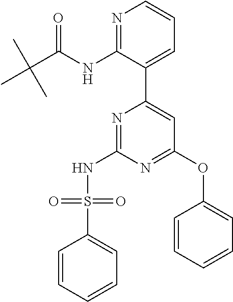 [SMILES: CC(C)(C)C(=O)Nc1ncccc1-c1cc(Oc2ccccc2)nc(NS(=O)(=O)c2ccccc2)n1] | 1.46 | 503.163 | 504 | A |
| 1265 | 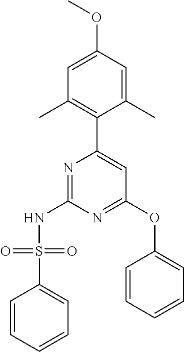 [SMILES: COc1cc(C)c(-c2cc(Oc3ccccc3)nc(NS(=O)(=O)c3ccccc3)n2)c(C)c1] | 1.86 | 461.141 | 462 | A |
| 1266 | 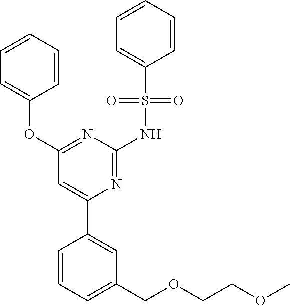 [SMILES: COCCOCc1cccc(-c2cc(Oc3ccccc3)nc(NS(=O)(=O)c3ccccc3)n2)c1] | 1.83 | 491.151 | 492 | A |
| 1267 | 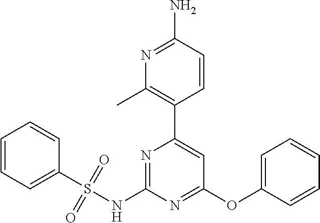 [SMILES: Cc1nc(N)ccc1-c1cc(Oc2ccccc2)nc(NS(=O)(=O)c2ccccc2)n1] | 1.16 | 433.121 | 434 | A |
| 1268 | 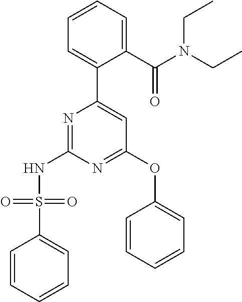 [SMILES: CCN(CC)C(=O)c1ccccc1-c1cc(Oc2ccccc2)nc(NS(=O)(=O)c2ccccc2)n1] | 1.75 | 502.167 | 503 | A |
| 1269 | 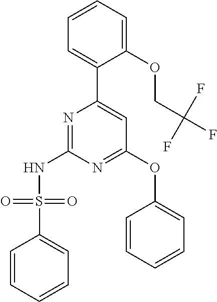 [SMILES: O=S(=O)(Nc1nc(Oc2ccccc2)cc(-c2ccccc2OCC(F)(F)F)n1)c1ccccc1] | 1.92 | 501.097 | 502 | A |
| 1270 | 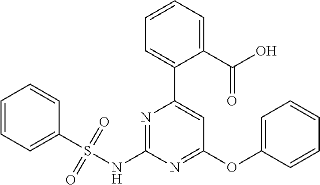 [SMILES: O=C(O)c1ccccc1-c1cc(Oc2ccccc2)nc(NS(=O)(=O)c2ccccc2)n1] | 1.54 | 447.089 | 448 | A |
| 1271 | 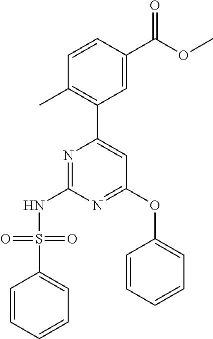 [SMILES: COC(=O)c1ccc(C)c(-c2cc(Oc3ccccc3)nc(NS(=O)(=O)c3ccccc3)n2)c1] | 1.89 | 475.12 | 476 | A |
| 1272 | 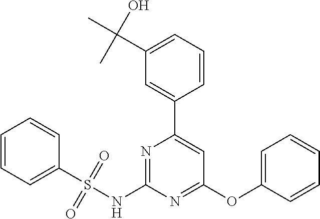 [SMILES: CC(C)(O)c1cccc(-c2cc(Oc3ccccc3)nc(NS(=O)(=O)c3ccccc3)n2)c1] | 1.74 | 461.141 | 462 | A |
| 1273 | 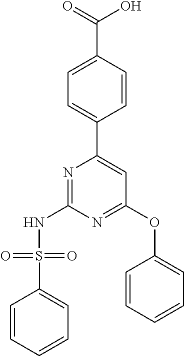 [SMILES: O=C(O)c1ccc(-c2cc(Oc3ccccc3)nc(NS(=O)(=O)c3ccccc3)n2)cc1] | 1.6 | 447.089 | 448 | A |
| 1274 | 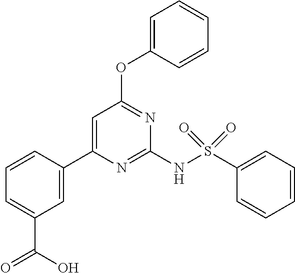 [SMILES: O=C(O)c1cccc(-c2cc(Oc3ccccc3)nc(NS(=O)(=O)c3ccccc3)n2)c1] | 1.62 | 447.089 | 448 | A |

| Compound | |
|---|---|
| number | NMR |
| 1223 | ¹H NMR (400 MHz, DMSO-d₆) δ 8.00 (d, J = 7.7 Hz, 1H), 7.85 (dd, J = 7.8, 4.7 Hz, 1H) 7.57 (t, J = 7.8 Hz |
| | 3H), 7.40 (dt, J = 25.8, 7.7 Hz, 5H), 7.24 (d, J = 8.4 Hz, 2H), 6.87 (s, 1H). |
| 1224 | ¹H NMR (400 MHz, DMSO-d₆) δ 11.56 (s, 1H), 7.58 (dd, J = 15.8, 7.6 Hz, 3H), 7.50 (t, J = 7.7 Hz, 2H), |
| | 7.44 (t, J = 7.6 Hz, 2H), 7.34 (t, J = 7.4 Hz, 1H), 7.16 (d, J = 8.4 Hz, 2H), 6.83 (s, 1H), 6.63 (s, 1H), 2.28 (t, |
| | J = 16.0 Hz, 2H), 2.04 (d, J = 11.7 Hz, 1H), 1.50 (t, J = 14.4 Hz, 1H), 1.38-1.16 (m, 2H), 0.86 (t, J = 6.6 Hz, |
| | 1H). |
| 1225 | ¹H NMR (400 MHz, DMSO-d₆) δ 11.92 (s, 1H), 8.59 (d, J = 7.4 Hz, 2H), 7.63-7.48 (m, 6H), 7.39 (t, J = 7.0 |
| | Hz, 3H), 7.26 (d, J = 8.3 Hz, 2H), 6.97 (s, 1H), 2.40 (s, 3H). |
| 1226 | ¹H NMR (400 MHz, DMSO-d₆) δ 11.96 (s, 1H), 8.81 (s, 1H), 8.77 (d, J = 5.5 Hz, 1H), 7.75 (d, J = 5.5 Hz, |
| | 1H), 7.62-7.49 (m, 5H), 7.40 (t, J = 7.7 Hz, 3H), 7.26 (d, J = 7.7 Hz, 2H), 7.03 (s, 1H), 2.40 (s, 3H). |
| 1227 | ¹H NMR (400 MHz, DMSO-d₆) δ 11.64 (s, 1H), 8.94 (s, 1H)), 8.71 (s, 1H), 7.54 (dt, J = 25.6, 22.6 Hz, 7H), |
| | 7.35 (t, J = 7.4 Hz, 1H), 7.18 (d, J = 8.7 Hz, 2H), 6.49 (s, 1H), 3.33 (d, J = 12.4 Hz, 2H), 3.01-2.88 (m, 2H), |
| | 2.83 (t, J = 11.3 Hz, 1H), 1.83 (dd, J = 42.2, 13.7 Hz, 4H). |
| 1233 | ¹H NMR (400 MHz, DMSO-d₆) δ 11.52 (s, 1H), 7.59 (s, 3H), 7.50 (t, J = 7.8 Hz, 2H), 7.43 (s, 2H), 7.34 (t, |
| | J = 7.2 Hz, 1H), 7.17 (d, J = 8.2 Hz, 2H), 6.89 (s, 1H), 6.59 (s, 1H), 2.22 (t, J = 28.1 Hz, 2H), 1.94 (d, J = |
| | 24.5 Hz, 2H), 1.25 (t, J = 11.5 Hz, 1H), 1.11 (d, J = 16.9 Hz, 1H), 0.88 (s, 9H). |
| 1236 | ¹H NMR (400 MHz, DMSO-d₆) δ 11.54 (s, 1H), 7.78-7.54 (m, 5H), 7.47 (dt, J = 13.4, 7.0 Hz, 4H), 7.37- |
| | 7.30 (m, 1H), 7.18 (d, J =8.2 Hz, 2H), 6.62 (d, J = 7.0 Hz, 2H), 2.04 (d, J = 29.2 Hz, 2H), 1.36 (s, 2H), 1.00 |
| | (d, J = 47.1 Hz, 12H). |
| 1238 | ¹H NMR (400 MHz, DMSO-d₆) δ 11.68 (s, 1H), 7.64-7.50 (m, 5H), 7.47-7.34 (m, 3H), 7.18 (d, J = 8.4 Hz, |
| | 2H), 6.46 (s, 1H), 4.16 (d, J = 11.2 Hz, 1H), 3.98 (d, J = 11.2 Hz, 1H), 1.83 (s, 2H), 1.66-1.45 (m, 3H), 1.24 |
| | (s, 1H). |
| 1239 | ¹H NMR (400 MHz, DMSO-d₆) δ 11.59 (s, 1H), 7.52 (t, J = 7.9 Hz, 5H), 7.36 (t, J = 7.3 Hz, 3H), 7.21-7.16 |
| | (m, 2H), 6.38 (s, 1H), 6.07 (s, 1H), 2.07 (s, 3H), 1.90 (s, 3H). |
| 1242 | ¹H NMR (400 MHz, DMSO-d₆) δ 11.57 (s, 1H), 7.67-7.45 (m, 5H), 7.38 (d, J = 6.4 Hz, 3H), 7.17 (d, J = 8.2 |
| | Hz, 2H), 6.44 (s, 1H), 1.71 (dd, J = 29.3, 10.8 Hz, 5H), 1.50-1.07 (m, 6H). |
| 1243 | ¹H NMR (400 MHz, DMSO-d₆) δ 11.60 (s, 1H), 7.53 (t, J = 7.7 Hz, 4H), 7.38 (d, J = 6.9 Hz, 3H), 7.18 (d, |
| | J = 7.7 Hz, 2H), 6.53 (d, J = 11.6 Hz, 1H), 3.90 (d, J = 10.9 Hz, 2H), 2.71 (d, J = 28.6, Hz, 1H), 1.63 (s, 4H). |
| 1245 | ¹H NMR (400 MHz, DMSO-d₆) δ 11.53 (s, 1H), 7.64-7.48 (m, 5H), 7.43 (t, J = 6.7 Hz, 2H), 7.34 (t, J = 7.4 |
| | Hz), 7.18 (d, J = 7.6 Hz, 2H), 6.87 (s, 1H), 6.59 (s, 1H), 2.27-2.17 (m, 4H), 1.69-1.53 (m, 4H). |
| 1246 | ¹H NMR (400 MHz, MeOD) δ 7.57-7.52 (m, 2H), 7.45-7.38 (m, 4H), 7.22 (dd, J = 16.1, 8.5 Hz, 4H), 6.76 |
| | (d, J = 8.8 Hz, 2H), 6.40 (s, 1H), 3.74 (s, 3H), 2.35 (s, 3H), 2.11 (s, 3H). |
| 1247 | ¹H NMR (400 MHz, DMSO-d₆) δ 8.12 (d, J = 5.2 Hz, 1H), 7.57 (dd, J = 13.5, 5.3 Hz, 3H), 7.42 (d, J = 7.3 |
| | Hz, 3H), 7.37-7.32 (m, 2H), 7.25 (d, J = 7.8 Hz, 2H), 6.98 (d, J = 5.2 Hz, 1H), 6.72 (s, 1H), 3.78 (s. 3H), |
| | 2.07 (s, 3H). |
| 1249 | ¹H NMR (400 MHz, DMSO-d₆) δ 11.69 (s, 1H), 7.51 (dd, J = 21.7, 9.5 Hz, 6H), 7.45-7.28 (m, 5H), 7.20 (d, |
| | J = 7.7 Hz, 2H), 7.01 (s, 1H), 6.75 (s, 1H), 6.41 (s, 1H), 1.26 (s, 2H), 0.65 (s, 2H). |
| 1250 | ¹H NMR (400 MHz, DMSO-d₆) δ 11.88 (s, 1H), 8.17 (s, 1H), 7.95 (d, J = 8.2 Hz, 1H), 7.85 (s, 1H), 7.70 (d, |
| | J = 7.7 Hz, 2H), 7.63-7.49 (m, 5H), 7.46-7.37 (m, 3H), 7.22 (d, J = 7.7 Hz, 2H), 2.08 (s, 1H). |
| 1251 | ¹H NMR (400 MHz, DMSO-d₆) δ 11.85 (s, 1H), 7.69-7.21 (m, 16H), 3.03 (s, 4H), 2.08 (s, 2H), 1.56 (s, 6H). |
| 1253 | ¹H NMR (400 MHz, DMSO-d₆) δ 11.86 (s, 1H), 8.27 (d, J = 6.5 Hz, 1H), 8.20 (d, J = 7.3 Hz, 1H), 7.80 (d, |
| | J = 7.4 Hz, 2H), 7.62-7.47 (m, 6H), 7.37 (t, J = 7.3 Hz, 1H), 7.26 (d, J = 8.0 Hz, 2H), 7.20-7.08 (m, 2H), |
| | 5.37-5.29 (m, 1H), 1.16 (d, J = 6.2 Hz, 6H). |
| 1255 | ¹H NMR (400 MHz, DMSO-d₆) δ 11.79 (s, 1H), 9.97 (s, 1H), 7.99 (d, J = 8.6 Hz, 1H), 7.77 (d, J = 8.6 Hz, |
| | 1H), 7.55 (t, J = 7.8 Hz, 5H), 7.44-7.34 (m, 3H), 7.25 (d, J = 8.3 Hz, 2H), 6.86 (s, 1H), 2.51 (s, 3H), 1.25 |
| | (s, 9H). |
| 1256 | ¹H NMR (400 MHz, DMSO-d₆) δ 11.71 (s, 1H), 8.77 (s, 1H), 8.03 (s, 1H), 7.65-7.50 (m, 6H), 7.44 (t, J = |
| | 7.5 Hz, 2H), 7.36 (t, J = 7.1 Hz, 1H), 7.26-7.21 (m, 3H), 3.74 (s, 4H), 3.23-3.20 (m, 4H), 2.31 (s, 3H). |
| 1257 | ¹H NMR (400 MHz, DMSO-d₆) δ 11.82 (s, 1H), 7.70-7.27 (m, 16H), 7.08 (s, 2H), 6.89 (d, J = 7.0 Hz, 2H), |
| | 5.82 (s, 1H). |
| 1258 | ¹H NMR (400 MHz, DMSO-d₆) δ 11.81 (s, 1H), 7.57 (dd, J = 14.2, 7.3 Hz, 5H), 7.49 (t, J = 6.5 Hz, 3H), |
| | 7.43-7.35 (m, 5H), 7.25 (d, J = 8.5 Hz, 2H), 6.89 (s, 1H), 5.10 (s, 1H), 4.53 (s, 2H), 3.17 (d, J = 6.0 Hz, 1H). |
| 1260 | ¹H NMR (400 MHz, DMSO-d₆) δ 11.75 (s, 1H), 8.0 (d, J = 8.1 Hz, 2H), 7.65 (d, J = 7.5 Hz, 2H), 7.61-7.51 |
| | (m, 3H), 7.45 (d, J = 7.9 Hz, 4H), 7.34 (t, J = 7.4 Hz, 1H), 7.23 (d, J = 8.4 Hz, 3H), 4.56 (s, 2H), 3.63-3.58 |
| | (m, 2H), 3.55-3.49 (m, 2H), 3.27 (s, 3H). |
| 1261 | ¹H NMR (400 MHz, DMSO-d₆) δ 11.79 (s, 1H), 10.34 (s, 1H), 7.89-7.79 (m, 2H), 7.56 (t, J = 7.7 Hz, 5H), |
| | 7.46-7.31 (m, 3H), 7.27 (s, 3H), 7.09 (s, 1H), 4.22 (d, J = 46.2, 2H), 3.85 (s, 3H), 3.49 (s, 1H), 2.54 (s, 3H), |
| | 1.32 (d, J = 6.3 Hz, 6H). |
| 1262 | ¹H NMR (400 MHz, DMSO-d₆) δ 11.73 (s, 1H), 7.94 (d, J = 8.4 Hz, 2H), 7.67-7.50 (m, 7H), 7.45 (t, J = 7.5 |
| | Hz, 2H), 7.37 (t, J = 7.4 Hz, 1H), 7.28-7.14 (m, 3H), 5.15 (s, 1H), 1.45 (s, 6H). |
| 1263 | ¹H NMR (400 MHz, DMSO-d₆) δ 11.88 (s, 1H), 7.61-7.56 (m, 2H), 7.56-7.51 (m, 5H), 7.45-7.36 (m, 3H), |
| | 7.24 (d, J = 8.6 Hz 2H), 7.07 (s, 1H), 6.92 (d, J = 1.9 Hz, 1H), 5.75 (dt, J = 13.0, 6.4 Hz, 1H), 1.33 (d, J = |
| | 6.5 Hz, 6H). |
| 1264 | ¹H NMR (400 MHz, DMSO-d₆) δ 11.83 (s, 1H), 9.97 (s, 1H), 8.53 (d, J = 4.8 Hz, 1H), 7.95 (d, J = 9.6 Hz, |
| | 1H), 7.60-7.52 (m, 3H), 7.48 (d, J = 6.7 Hz, 2H), 7.40 (dt, J = 12.9, 6.4 Hz, 4H), 7.16 (d, J = 7.6 Hz, 2H), |
| | 6.83 (s, 1H), 1.11 (s, 9H). |
| 1267 | ¹H NMR (400 MHz, DMSO-d₆) δ 14.48 (s, 1H), 11.89 (s, 1H), 7.95 (d, J = 9.0 Hz, 1H), 7.56 (dd, J = 17.5, |
| | 9.9 Hz, 5H), 7.42 (d, J = 7.7 Hz, 3H), 7.23 (d, J = 8.3 Hz, 2H), 6.92 (d, J = 7.0 Hz, 2H), 2.57 (s, 3H). |
| 1268 | ¹H NMR (400 MHz, DMSO-d₆) δ 11.73 (s, 1H), 7.64-7.52 (m, 8H), 7.38 (t, J = 14.4 Hz, 4H), 7.17 (d, J = 7.7 |
| | (d, J = 7.7 Hz, 2H), 6.67 (s, 1H), 2.90 (d, J = 6.9 Hz, 2H), 0.99 (s, 3H), 0.78 (s, 3H). |
| 1270 | ¹H NMR (400 MHz, DMSO-d₆) δ 12.99 (s, 1H), 11.71 (s, 1H), 7.83 (s, 1H), 7.68-7.51 (m, 6H), 7.48-7.31 |
| | (m, 6H), 7.22 (d, J = 8.4 Hz, 2H), 6.72 (s, 1H). |
| 1271 | ¹H NMR (400 MHz, DMSO-d₆) δ 11.82 (s, 1H), 7.95 (d, J = 5.7 Hz, 2H), 7.60-7.46 (m, 6H), 7.38 (t, J = 6.1 |
| | Hz, 3H), 7.25 (d, J = 8.2 Hz, 2H), 6.84 (s, 1H), 3.87 (s, 3H), 2.38 (s, 3H). |
| 1272 | ¹H NMR (400 MHz, DMSO-d₆) δ 11.75 (s, 1H), 8.19 (s, 1H), 7.84 (d, J = 7.7 Hz, 1H), 7.66 (dd, J = 12.1, 7.8 |
| | Hz, 3H), 7.55 (dt, J = 15.4, 7.5 Hz, 3H), 7.44 (t, J = 7.7 Hz, 3H), 7.37 (t, J = 7.3 Hz, 1H)), 7.24 (d, J = 8.5 Hz, |
| | 3H), 2.08 (s, 1H), 1.48 (s, 6H). |
| 1274 | ¹H NMR (400 MHz, DMSO-d₆) δ 11.82 (s, 1H), 8.70 (s, 1H), 8.27 (d, J = 7.0 Hz, 1H), 8.09 (d, J = 7.8 Hz, |
| | 1H), 7.72-7.62 (m, 3H), 7.56 (dt, J = 15.2, 7.7 Hz, 3H), 7.44 (t, J = 7.5 Hz, 2H), 7.36 (dd, J = 15.7, 8.8 Hz, |
| | 2H), 7.25 (d, J = 8.4 Hz, 2H). |

### Example 341: Preparation of Compound 1275 and Compound 1276

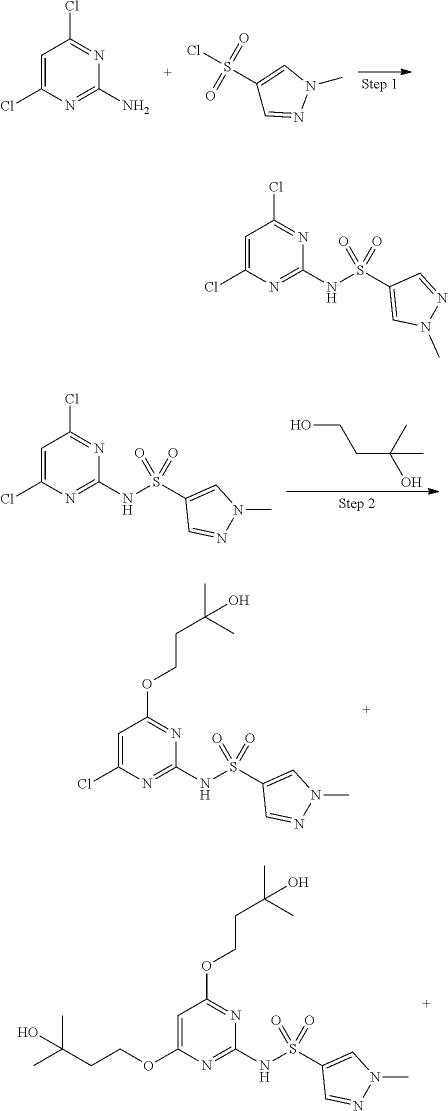

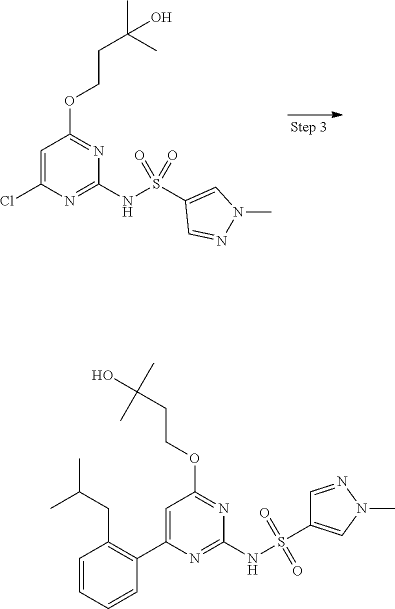

### Step 1: N-(4,6-Dichloropyrimidin-2-yl)-1-methyl-pyrazole-4-sulfonamide

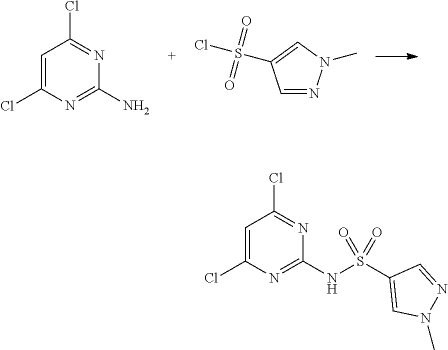

To a THF (23 mL) suspension of sodium hydride (580.3 mg, 24.18 mmol) was added 4,6-dichloropyrimidin-2-amine (1.0081 g, 6.147 mmol) in an ice-water bath and then the reaction was warmed to room temperature and stirred for 15 minutes. The reaction was cooled to 0° C. and a THF (7 mL) solution of 1-methylpyrazole-4-sulfonyl chloride (1.6962 g, 9.391 mmol) was added dropwise via syringe. The reaction mixture was stirred for 15 minutes at 0° C. and then quenched with HCl (approximately 24.59 mL of 1 M, 24.59 mmol). Ethyl acetate (25 mL) was added and the organic layer was separated, dried with anhydrous sodium sulfate, filtered, and concentrated in vacuo. The crude residue was taken up in EtOH (6 mL) and stirred at 0° C. for 30 minutes upon which the product crystallized out. Filtration and drying gave N-(4,6-dichloropyrimidin-2-yl)-1-methyl-pyrazole-4-sulfonamide (1.668 g, 88%)¹H NMR (400 MHz, DMSO-d₆) δ 12.41 (s, 1H), 8.41 (s, 1H), 7.91 (d, J=0.8 Hz, 1H), 7.55 (s, 1H), 3.88 (s, 3H). ESI-MS m/z calc. 306.96976, found 308.22 (M+1)⁺; Retention time: 0.49 minutes (LC method D).

### Step 2: N-[4-chloro-6-(3-hydroxy-3-methyl-butoxy)pyrimidin-2-yl]-1-methyl-pyrazole-4-sulfonamide and N-[4,6-bis(3-hydroxy-3-methyl-butoxy)pyrimidin-2-yl]-1-methyl-pyrazole-4-sulfonamide (Compound 1276)

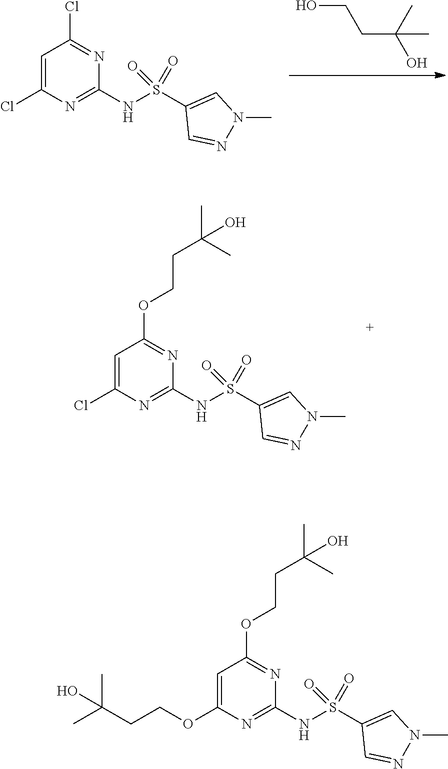

NaH (approximately 583.9 mg of 60% w/w, 14.60 mmol) was added to 3-methylbutane-1,3-diol (approximately 1.521 g, 14.60 mmol) in DMF (15 mL). The mixture was stirred at room temperature for 30 minutes. To the mixture was then added dropwise to N-(4,6-dichloropyrimidin-2-yl)-1-methyl-pyrazole-4-sulfonamide (3 g, 9.736 mmol) in DMF (30.00 mL) and the combined mixture was stirred at room temperature for 30 minutes. More 3-methylbutane-1,3-diol (1 g, stirred in NaH and DMF) was added dropwise to reaction mixture. After another 30 minutes. at room temperature, the reaction mixture was cooled down with ice/water, and the pH of the reaction mixture was adjusted to ˜4 with 1 N HCl, extracted with ethyl acetate (3×20 ml). The combined organic layers were extracted with brine, dried over Na₂SO₄, concentrated, and purified on silica using a gradient of ethyl acetate and hexane. Two products were isolated: N-[4-chloro-6-(3-hydroxy-3-methyl-butoxy)pyrimidin-2-yl]-1-methyl-pyrazole-4-sulfonamide (3.04 g); ESI-MS m/z calc. 375.08, found 376.1 (M+1)⁺; Retention time: 1.11 minutes (LC method A) and side product N-[4,6-bis(3-hydroxy-3-methyl-butoxy)pyrimidin-2-yl]-1-methyl-pyrazole-4-sulfonamide (9.4 mg)¹H NMR (400 MHz, DMSO) δ 11.35 (s, 1H), 8.34 (s, 1H), 7.85 (s, 1H), 5.74 (s, 1H), 4.31-4.26 (m, 9H), 3.87 (s, 3H), 1.78 (t, J=7.3 Hz, 4H), 1.14 (s, 12H). ESI-MS m/z calc. 443.18387, found 444.4 (M+1)⁺; Retention time: 1.09 minutes (LC method A).

### Step 3: N-[4-(3-hydroxy-3-methyl-butoxy)-6-(2-isobutylphenyl)pyrimidin-2-yl]-1 -methyl-pyrazole-4-sulfonamide (Compound 1275)

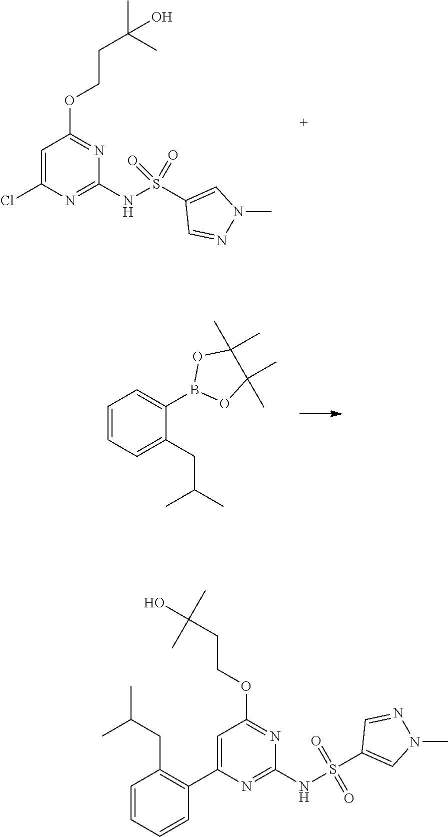 [SMILES: Cn1cc(S(=O)(=O)Nc2nc(Cl)cc(OCCC(C)(C)O)n2)cn1 ; CC(C)Cc1ccccc1B1OC(C)(C)C(C)(C)O1 ; CC(C)Cc1ccccc1-c1cc(OCCC(C)(C)O)nc(NS(=O)(=O)c2cnn(C)c2)n1]

N-[4-chloro-6-(3-hydroxy-3-methyl-butoxy)pyrimidin-2-yl]-1-methyl-pyrazole-4-sulfonamide (30 mg, 0.080 mmol) 2-(2-isobutylphenyl)-4,4,5,5-tetramethyl-1,3,2-dioxaborolane (42 mg, 0.16 mmol), sodium carbonate (160 μL of 2 M aqueous solution, 0.32 mmol) and Pd(dppf)Cl₂ (12 mg, 0.016 mmol) were combined with DMF (0.5 mL) in a vial. The reaction was purged with nitrogen and the reaction was stirred at 110° C. for 6 hours. The reaction mixture was filtered and purified by reverse phase HPLC (10-6₀% gradient of ACN in water containing HCl as a modifier). N-[4-(3-hydroxy-3-methyl-butoxy)-6-(2-isobutylphenyl)pyrimidin-2-yl]-1-methyl-pyrazole-4-sulfonamide (13.7 mg, 50%). ESI-MS m z calc. 473.2097, found 474.0 (M+1)⁺; Retention time: 1.58 minutes; LC method A.

### Example 342: Preparation of Compound 1277

### Step 1: N-[4-(2-cyclobutylphenyl)-6-(3-hydroxy-3-methyl-butoxy)pyrimidin-2-yl]-1 -methyl-pyrazole-4-sulfonamide

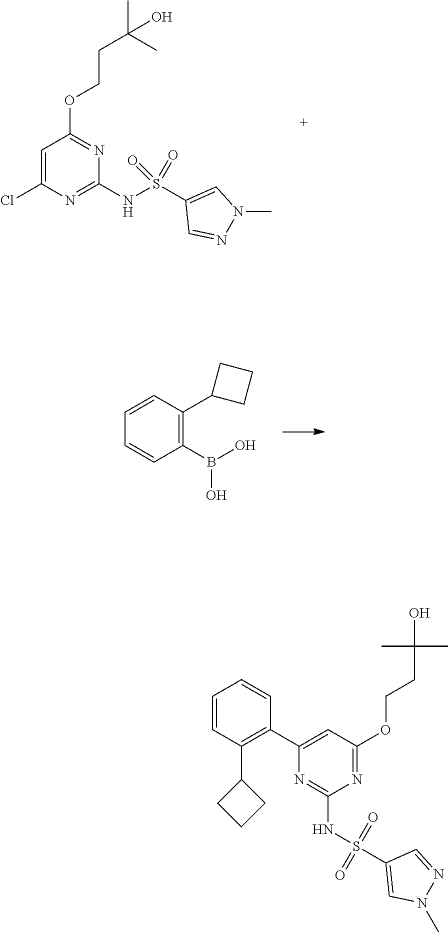 [SMILES: Cn1cc(S(=O)(=O)Nc2nc(Cl)cc(OCCC(C)(C)O)n2)cn1 ; OB(O)c1ccccc1C1CCC1 ; Cn1cc(S(=O)(=O)Nc2nc(OCCC(C)(C)O)cc(-c3ccccc3C3CCC3)n2)cn1]

The compound was prepared in a manner analogous to that described above using commercially available (2-cyclobutylphenyl)boronic acid to give N-[4-(2-cyclobutylphenyl)-6-(3-hydroxy-3-methyl-butoxy)pyrimidin-2-yl]-1-methyl-pyrazole-4-sulfonamide (20.5 mg, 65%). ESI-MS m/z calc. 471.19403, found 472.0 (M+1)⁺; Retention time: 1.52 minutes; LC method A.

### Example 343: Preparation of Compound 1278

### Step 1: N-[4-(3-hydroxy-3-methyl-butoxy)-6-(4-pentoxyphenyl)pyrimidin-2-yl]-1 -methyl-pyrazole-4-sulfonamide

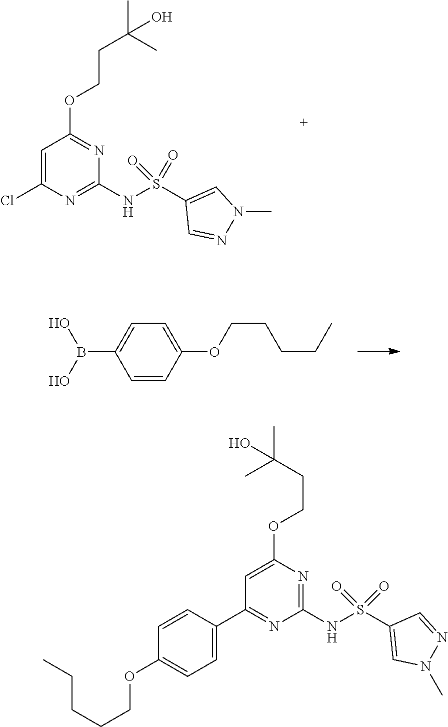

The compound was prepared in a manner analogous to that described above using commercially available (4-pentoxyphenyl)boronic acid to give N-[4-(3-hydroxy-3-methyl-butoxy)-6-(4-pentoxyphenyl)pyrimidin-2-yl]-1-methyl-pyrazole-4-sulfonamide (1.1 mg, 4%). ESI-MS m/z calc. 503.22025, found 504.2 (M+1)⁺; Retention time: 1.8 minutes; LC method A.

### Example 344: Preparation of Compound 1279 and Compound 1280

### Step 1: N-[4-(6,6-Dimethylcyclohexen-1-yl)-6-(3-hydroxy-3-methyl-butoxy)pyrimidin-2-yl]-1-methyl-pyrazole-4-sulfonamide (Compound 1279) and N-[4-(2,2-dimethylcyclohexyl)-6-(3-hydroxy-3-methyl-butoxy)pyrimidin-2-yl]-1 -methyl-pyrazole-4-sulfonamide (Compound 1280)

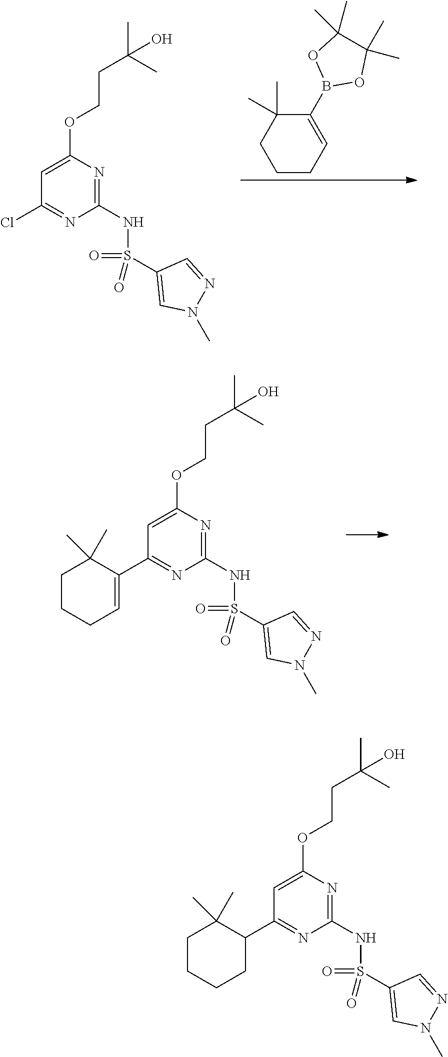

Stage 1: To a mixture of N-[4-chloro-6-(3-hydroxy-3-methyl-butoxy)pyrimidin-2-yl]-1-methyl-pyrazole-4-sulfonamide (63.6 mg, 0.1692 mmol), 2-(6,6-dimethylcyclohexen-1-yl)-4,4,5,5-tetramethyl-1,3,2-dioxaborolane (72.4 mg, 0.3066 mmol), sodium carbonate (300 μL of 2 M, 0.6000 mmol) in DMSO (0.5 mL) was added Pd(dppf)C₁₂ (24 mg). The mixture was thoroughly flushed with nitrogen and heated at 110° C. for 6 hours. The reaction mixture was filtered and purified on reverse phase HPLC (HCl modifier, 25-75% ACN-H₂O) to give N-[4-(6,6-dimethylcyclohexen-1-yl)-6-(3-hydroxy-3-methyl-butoxy)pyrimidin-2-yl]-1-methyl-pyrazole-4-sulfonamide (15.2 mg, 20%). ¹H NMR (400 MHz, DMSO) δ 8.27 (s, 1H), 7.79 (s, 1H), 6.23 (s, 1H), 5.92 (s, 1H), 4.35 (t, J=7.3 Hz, 2H), 3.89-3.82 (m, 3H), 2.54 (s, 1H), 2.07 (t, J=9.7 Hz, 3H), 1.84-1.75 (m, 2H), 1.64 (d, J=8.5 Hz, 3H), 1.50-1.40 (m, 2H), 1.16 (s, 5H), 1.08 (s, 6H). ESI-MS m/z calc. 449.2097, found 450.0 (M+1)⁺; Retention time: 1.46 minutes (LC method A).

Stage 2: The product from stage 1 was dissolved in methanol (10 mL) and Pd/C (35 mg of 5% w/w, 0.01644 mmol) was added. The mixture was stirred under a balloon of hydrogen for 40 hours. The mixture was filtered, concentrated under reduced pressure, and purified on reverse phase HPLC (HCl modifier, 25-75% ACN-H₂O) to give N-[4-(2,2-dimethylcyclohexyl)-6-(3-hydroxy-3-methyl-butoxy)pyrimidin-2-yl]-1-methyl-pyrazole-4-sulfonamide (4.2 mg, 5% ) ESI-MS m/z calc. 451.2253, found 452.0 (M+1)⁺; Retention time: 1.43 minutes (LC method A).

### Example 345: Characterization of Compounds 1281-1317

The compounds in the following tables were prepared in a manner analogous to that described above using commercially available reagents and intermediates described herein.

| | | LCMS | | | |
|---|---|---|---|---|---|
| Compound | | Rt | Calc. | | LCMS |
| number | Structure | (min) | mass | M + 1 | Method |
| 1281 | 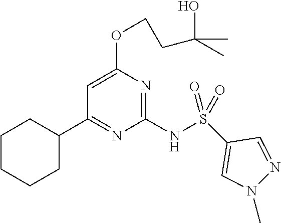 [SMILES: Cn1cc(S(=O)(=O)Nc2nc(OCCC(C)(C)O)cc(C3CCCCC3)n2)cn1] | 1.25 | 423.194 | 424.3 | A |
| 1282 | 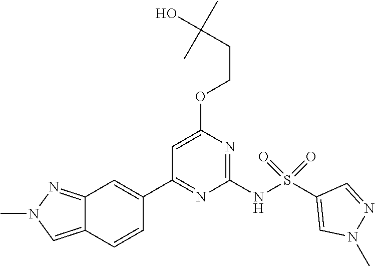 | 1.17 | 471.169 | 472.16 | A |
| 1283 | 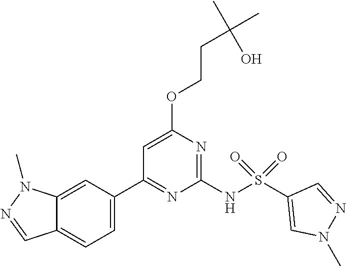 [SMILES: Cn1cc(S(=O)(=O)Nc2nc(OCCC(C)(C)O)cc(-c3ccc4cnn(C)c4c3)n2)cn1] | 1.27 | 471.169 | 472.16 | A |
| 1284 | 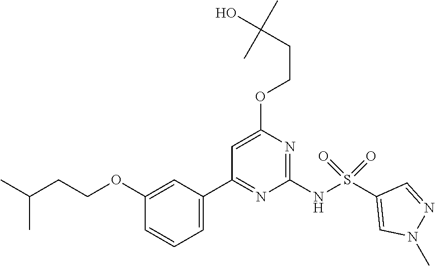 [SMILES: CC(C)CCOc1cccc(-c2cc(OCCC(C)(C)O)nc(NS(=O)(=O)c3cnn(C)c3)n2)c1] | 1.8 | 503.22 | 504.2 | A |
| 1285 | 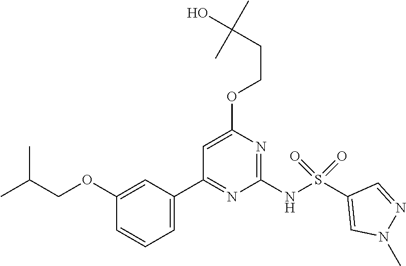 [SMILES: CC(C)COc1cccc(-c2cc(OCCC(C)(C)O)nc(NS(=O)(=O)c3cnn(C)c3)n2)c1] | 1.7 | 489.205 | 490.13 | A |
| 1286 | 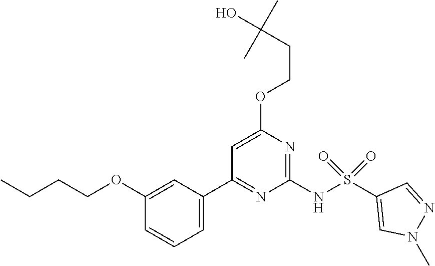 [SMILES: CCCCOc1cccc(-c2cc(OCCC(C)(C)O)nc(NS(=O)(=O)c3cnn(C)c3)n2)c1] | 1.3 | 489.205 | 490.13 | A |
| 1287 | 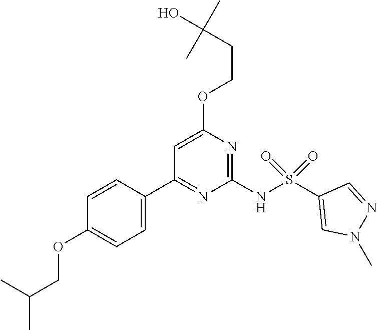 [SMILES: CC(C)COc1ccc(-c2cc(OCCC(C)(C)O)nc(NS(=O)(=O)c3cnn(C)c3)n2)cc1] | 1.68 | 489.205 | 490.13 | A |
| 1288 | 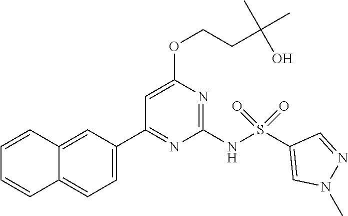 [SMILES: Cn1cc(S(=O)(=O)Nc2nc(OCCC(C)(C)O)cc(-c3ccc4ccccc4c3)n2)cn1] | 1.55 | 467.163 | 468.13 | A |
| 1289 | 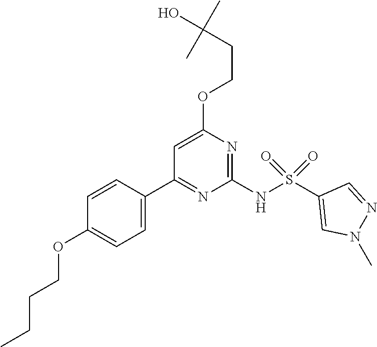 [SMILES: CCCCOc1ccc(-c2cc(OCCC(C)(C)O)nc(NS(=O)(=O)c3cnn(C)c3)n2)cc1] | 1.68 | 489.205 | 490.13 | A |
| 1290 | 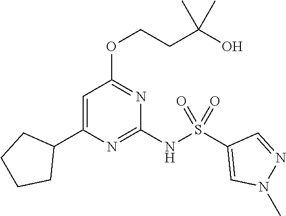 [SMILES: Cn1cc(S(=O)(=O)Nc2nc(OCCC(C)(C)O)cc(C3CCCC3)n2)cn1] | 1.15 | 409.178 | 410.5 | A |
| 1291 | 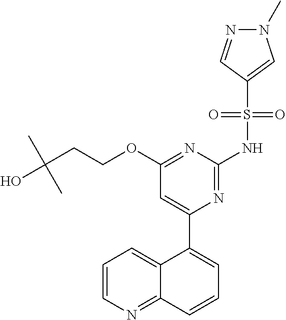 [SMILES: Cn1cc(S(=O)(=O)Nc2nc(OCCC(C)(C)O)cc(-c3cccc4ncccc34)n2)cn1] | 0.85 | 468.158 | 469 | A |
| 1292 | 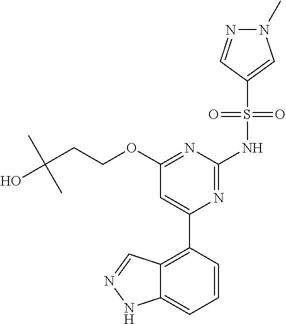 [SMILES: Cn1cc(S(=O)(=O)Nc2nc(OCCC(C)(C)O)cc(-c3cccc4[nH]ncc34)n2)cn1] | 1.1 | 457.153 | 458 | A |
| 1293 | 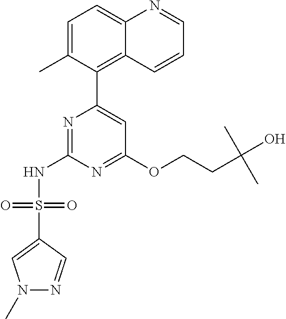 [SMILES: Cc1ccc2ncccc2c1-c1cc(OCCC(C)(C)O)nc(NS(=O)(=O)c2cnn(C)c2)n1] | 0.84 | 482.174 | 483 | A |
| 1294 | 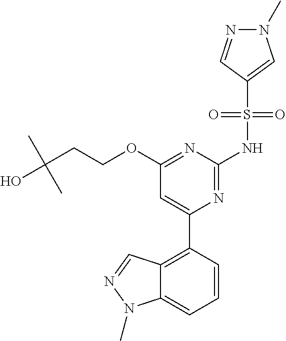 [SMILES: Cn1cc(S(=O)(=O)Nc2nc(OCCC(C)(C)O)cc(-c3cccc4c3cnn4C)n2)cn1] | 1.25 | 471.169 | 472 | A |
| 1295 | 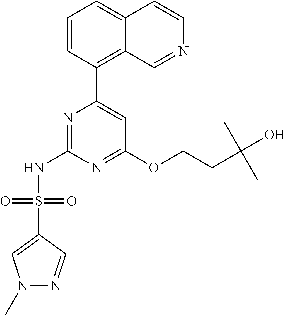 [SMILES: Cn1cc(S(=O)(=O)Nc2nc(OCCC(C)(C)O)cc(-c3cccc4ccncc34)n2)cn1] | 0.83 | 468.158 | 469 | A |
| 1296 | 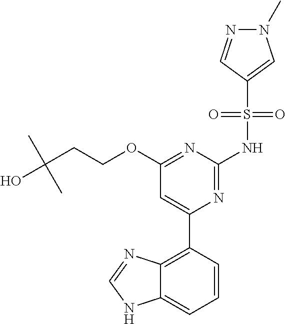 [SMILES: Cn1cc(S(=O)(=O)Nc2nc(OCCC(C)(C)O)cc(-c3cccc4[nH]cnc34)n2)cn1] | 0.89 | 457.153 | 458 | A |
| 1297 | 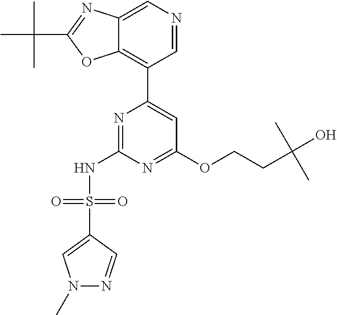 [SMILES: Cn1cc(S(=O)(=O)Nc2nc(OCCC(C)(C)O)cc(-c3cncc4nc(C(C)(C)C)oc34)n2)cn1] | 1.24 | 515.195 | 516 | A |
| 1298 | 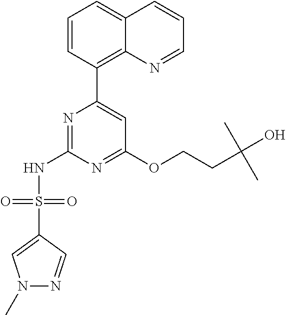 [SMILES: Cn1cc(S(=O)(=O)Nc2nc(OCCC(C)(C)O)cc(-c3cccc4cccnc34)n2)cn1] | 0.91 | 468.158 | 469 | A |
| 1299 | 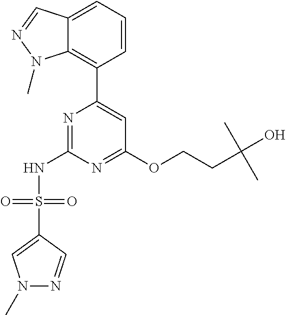 [SMILES: Cn1cc(S(=O)(=O)Nc2nc(OCCC(C)(C)O)cc(-c3cccc4cnn(C)c34)n2)cn1] | 1.22 | 471.169 | 472 | A |
| 1300 | 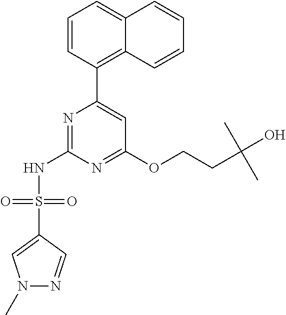 [SMILES: Cn1cc(S(=O)(=O)Nc2nc(OCCC(C)(C)O)cc(-c3cccc4ccccc34)n2)cn1] | 1.44 | 467.163 | 468 | A |
| 1301 | 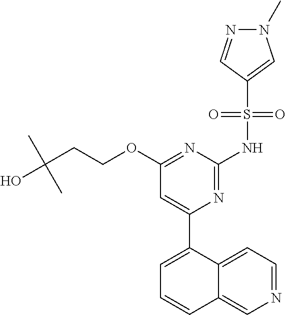 [SMILES: Cn1cc(S(=O)(=O)Nc2nc(OCCC(C)(C)O)cc(-c3cccc4cnccc34)n2)cn1] | 0.79 | 468.158 | 469 | A |
| 1302 | 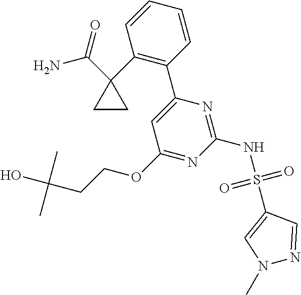 [SMILES: Cn1cc(S(=O)(=O)Nc2nc(OCCC(C)(C)O)cc(-c3ccccc3C3(C(N)=O)CC3)n2)cn1] | 1.03 | 500.184 | 501 | A |
| 1303 | 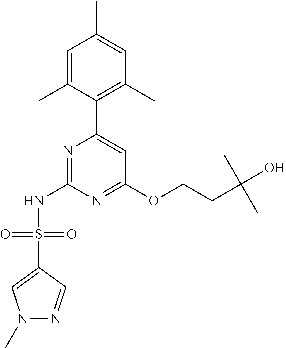 [SMILES: Cc1cc(C)c(-c2cc(OCCC(C)(C)O)nc(NS(=O)(=O)c3cnn(C)c3)n2)c(C)c1] | 1.42 | 459.194 | 460 | A |
| 1304 | 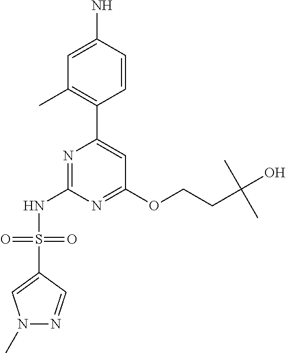 [SMILES: Cc1cc(N)ccc1-c1cc(OCCC(C)(C)O)nc(NS(=O)(=O)c2cnn(C)c2)n1] | 0.84 | 446.174 | 447 | A |
| 1305 | 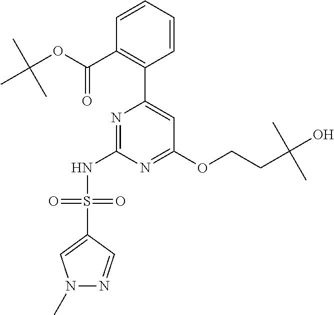 [SMILES: Cn1cc(S(=O)(=O)Nc2nc(OCCC(C)(C)O)cc(-c3ccccc3C(=O)OC(C)(C)C)n2)cn1] | 1.43 | 517.2 | 518 | A |
| 1306 | 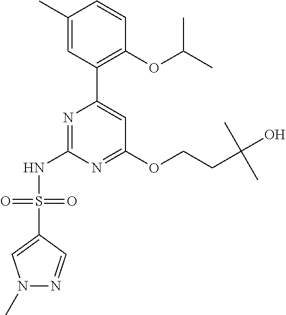 [SMILES: Cc1ccc(OC(C)C)c(-c2cc(OCCC(C)(C)O)nc(NS(=O)(=O)c3cnn(C)c3)n2)c1] | 1.5 | 489.205 | 490 | A |
| 1307 | 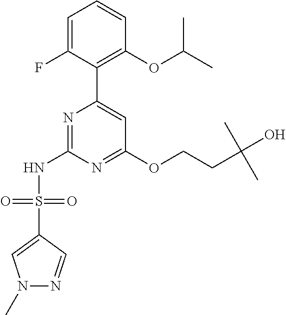 [SMILES: CC(C)Oc1cccc(F)c1-c1cc(OCCC(C)(C)O)nc(NS(=O)(=O)c2cnn(C)c2)n1] | 1.38 | 493.18 | 494 | A |
| 1308 | 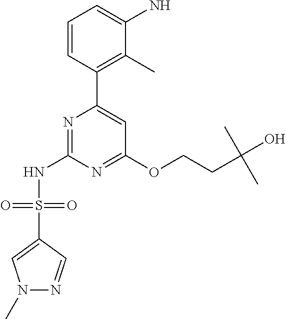 [SMILES: Cc1c(N)cccc1-c1cc(OCCC(C)(C)O)nc(NS(=O)(=O)c2cnn(C)c2)n1] | 0.83 | 446.174 | 447 | A |
| 1309 | 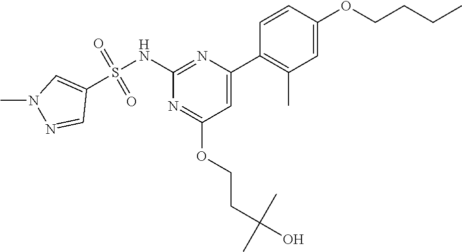 [SMILES: CCCCOc1ccc(-c2cc(OCCC(C)(C)O)nc(NS(=O)(=O)c3cnn(C)c3)n2)c(C)c1] | 1.7 | 503.22 | 504 | A |
| 1310 | 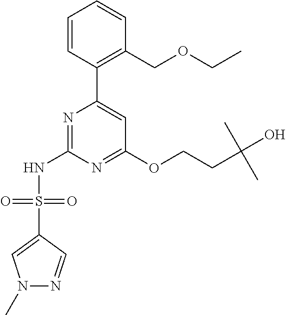 [SMILES: CCOCc1ccccc1-c1cc(OCCC(C)(C)O)nc(NS(=O)(=O)c2cnn(C)c2)n1] | 1.28 | 475.189 | 476 | A |
| 1311 | 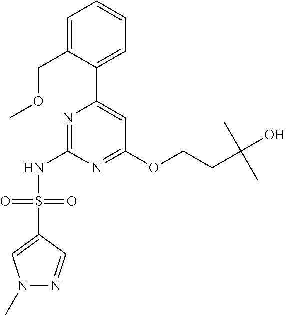 [SMILES: COCc1ccccc1-c1cc(OCCC(C)(C)O)nc(NS(=O)(=O)c2cnn(C)c2)n1] | 1.2 | 461.173 | 462 | A |
| 1312 | 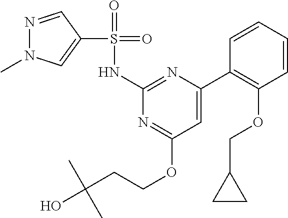 [SMILES: Cn1cc(S(=O)(=O)Nc2nc(OCCC(C)(C)O)cc(-c3ccccc3OCC3CC3)n2)cn1] | 1.43 | 487.189 | 488 | A |
| 1313 | 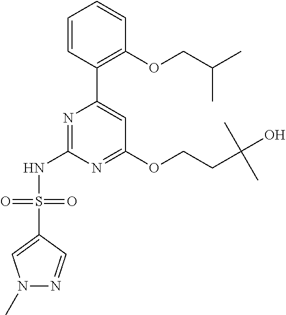 [SMILES: CC(C)COc1ccccc1-c1cc(OCCC(C)(C)O)nc(NS(=O)(=O)c2cnn(C)c2)n1] | 1.54 | 489.205 | 490 | A |
| 1314 | 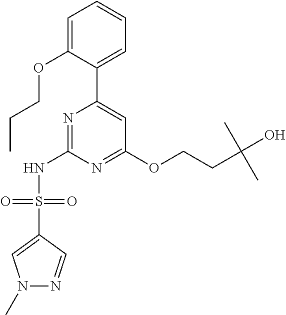 [SMILES: CCCOc1ccccc1-c1cc(OCCC(C)(C)O)nc(NS(=O)(=O)c2cnn(C)c2)n1] | 1.43 | 475.189 | 476 | A |
| 1315 | 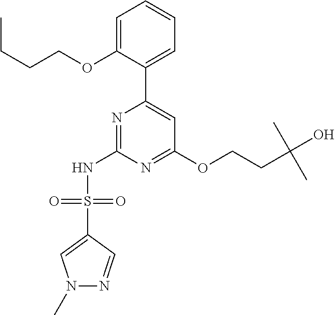 [SMILES: CCCCOc1ccccc1-c1cc(OCCC(C)(C)O)nc(NS(=O)(=O)c2cnn(C)c2)n1] | 1.54 | 489.205 | 490 | A |
| 1316 | 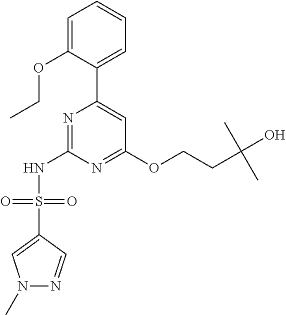 [SMILES: CCOc1ccccc1-c1cc(OCCC(C)(C)O)nc(NS(=O)(=O)c2cnn(C)c2)n1] | 1.3 | 461.173 | 462 | A |
| 1317 | 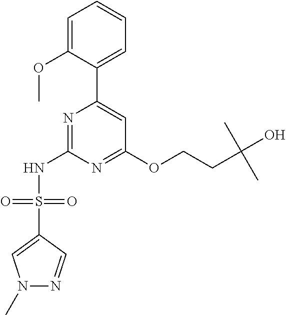 [SMILES: COc1ccccc1-c1cc(OCCC(C)(C)O)nc(NS(=O)(=O)c2cnn(C)c2)n1] | 1.18 | 447.158 | 448 | A |

### Example 346: Preparation of Compound 1318

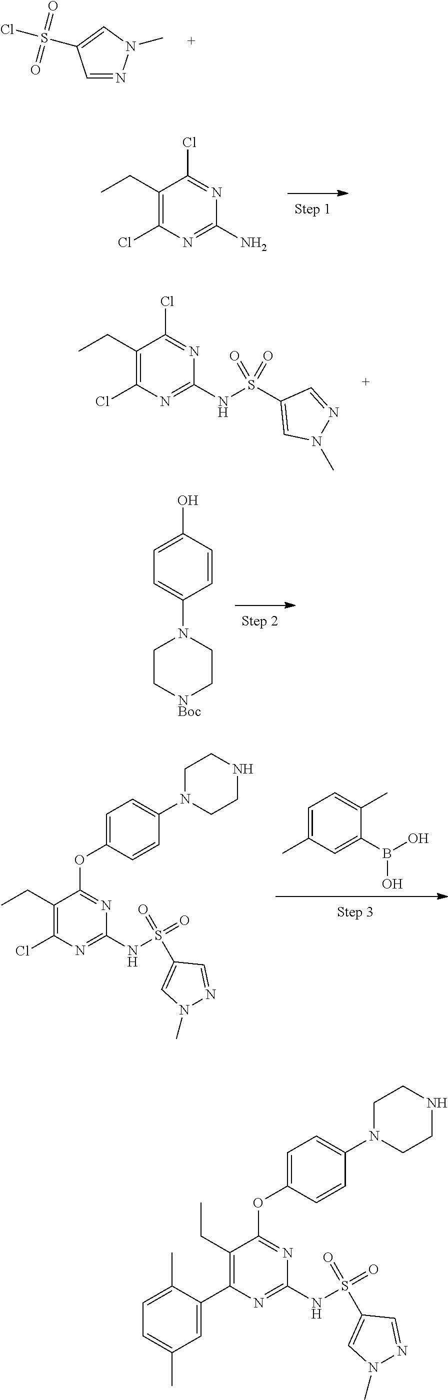

### Step 1: N-(4,6-Dichloro-5-ethyl-pyrimidin-2-yl)-1-methyl-pyrazole-4-sulfonamide

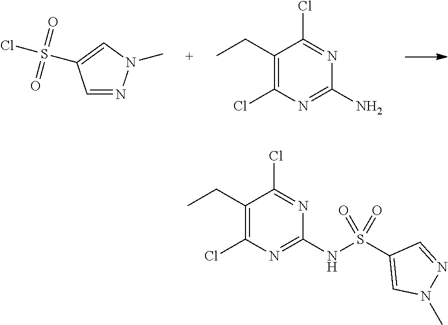

To a solution of 4,6-dichloro-5-ethyl-pyrimidin-2-amine (2.66 g, 13.85 mmol) in DMF (55 mL) at 0° C. was added sodium hydride (1.33 g, 55.4 mmol) and the reaction mixture was stirred at this temperature for 5 minutes, then removed from the cooling bath and stirred at room temperature for 10 minutes. The reaction mixture was cooled to 0° C. and a solution of 1-methylpyrazole-4-sulfonyl chloride (5 g, 27.68 mmol) in DMF (3.0 mL) was added dropwise over 1 minutes. The reaction mixture was stirred at this temperature for 5 minutes, then removed from the cooling bath and stirred at room temperature for 12 minutes. The reaction mixture was cooled back to 0° C. and quenched with hydrochloric acid (6.8 mL of 37% w/v, 69.0 mmol), then diluted with a solution of ethyl acetate/hexanes (1:1) and partitioned with a saturated aqueous solution of sodium bicarbonate. The organic layer was removed, and the aqueous layer was further extracted with ethyl acetate/hexanes (1:1, 5×). The combined organic extracts were dried with brine and magnesium sulfate. The solution was filtered, and the filtrate was concentrated in vacuo. The crude residue was purified by flash column chromatography on silica gel (5 to 40% ethyl acetate in hexanes). N-(4,6-dichloro-5-ethyl-pyrimidin-2-yl)-1-methyl-pyrazole-4-sulfonamide (4.09 g, 88%) as isolated as a white solid. ESI-MS m/z calc. 341.07132, found 342.3 (M+1)⁺; Retention time: 0.55 minutes (LC method D). ¹H NMR (400 MHz, DMSO-d₆) δ 12.24 (s, 1H), 8.40 (s, 1H), 7.91 (d, J=0.7 Hz, 1H), 3.88 (s, 3H), 2.71 (q, J=7.5 Hz, 2H), 1.10 (t, J=7.5 Hz, 3H).

### Step 2: N-[4-chloro-5-ethyl-6-(4-piperazin-1-ylphenoxy)pyrimidin-2-yl]-1-methyl-pyrazole-4-sulfonamide

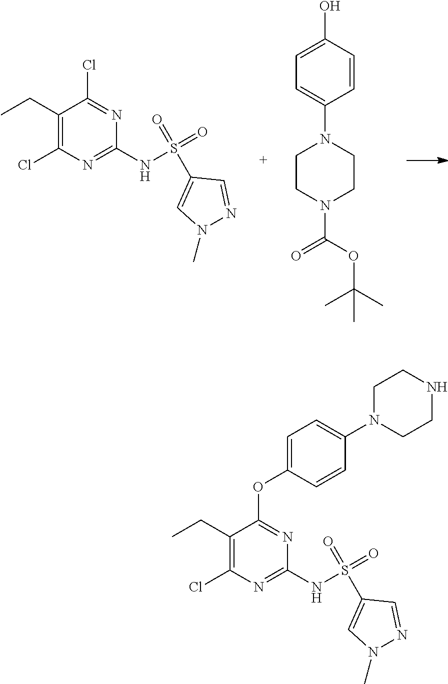 [SMILES: CCc1c(Cl)nc(NS(=O)(=O)c2cnn(C)c2)nc1Cl ; CC(C)(C)OC(=O)N1CCN(c2ccc(O)cc2)CC1 ; CCc1c(Cl)nc(NS(=O)(=O)c2cnn(C)c2)nc1Oc1ccc(N2CCNCC2)cc1]

Stage 1: A heterogeneous solution of N-(4,6-dichloro-5-ethyl-pyrimidin-2-yl)-1 -methyl-pyrazole-4-sulfonamide (1.5 g, 4.462 mmol), tert-butyl 4-(4-hydroxyphenyl)piperazine-1-carboxylate (1.24 g, 4.455 mmol), and cesium carbonate (4.36 g, 13.4 mmol) in NMP (28 mL) was heated in a sealed pressure vessel to 110° C. for 16 hours. The reaction mixture was cooled and diluted with water (150 mL). The reaction mixture was acidified with acetic acid (1.60 g, 26.6 mmol) and the solution was extracted three times with ethyl acetate/hexanes (1:1). The organic layer was washed with brine, dried over magnesium sulfate, filtered, and concentrated in vacuo onto silica gel. The crude impregnated silica gel was subjected to flash column chromatography (0 to 60% ethyl acetate in hexanes).

Stage 2: The semi-pure residue from stage one was dissolved in dioxane (9.0 mL) and HCl (6.7 mL of 4 M, 26.8 mmol) (in dioxane) was slowly added. The reaction mixture was stirred for 140 minutes before removing the solvent in vacuo. N-[4-chloro-5-ethyl-6-(4-piperazin-1-ylphenoxy)pyrimidin-2-yl]-1-methyl-pyrazole-4-sulfonamide (hydrochloride salt) (1.5 g, 54%) was isolated as a yellow hydroscopic solid. ESI-MS m/z calc. 477.13498, found 478.48 (M+1)⁺; Retention time: 0.47 minutes, LC method D.

### Step 3: N-[4-(2,5-Dimethylphenyl)-5-ethyl-6-(4-piperazin-1-ylphenoxy)pyrimidin-2-yl]-1-methyl-pyrazole-4-sulfonamide

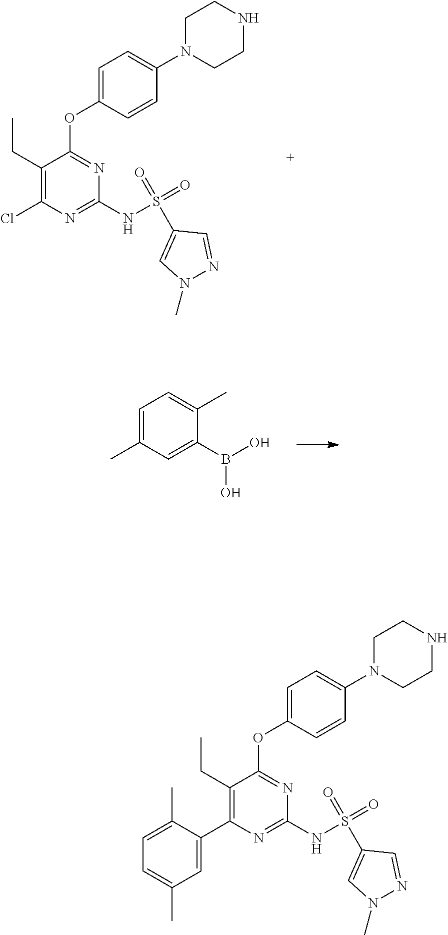 [SMILES: CCc1c(Cl)nc(NS(=O)(=O)c2cnn(C)c2)nc1Oc1ccc(N2CCNCC2)cc1 ; Cc1ccc(C)c(B(O)O)c1 ; CCc1c(Oc2ccc(N3CCNCC3)cc2)nc(NS(=O)(=O)c2cnn(C)c2)nc1-c1cc(C)ccc1C]

A heterogeneous mixture of (2,5-dimethylphenyl)boronic acid (approximately 44.99 mg, 0.3000 mmol), N-[4-chloro-5-ethyl-6-(4-piperazin-1-ylphenoxy)pyrimidin-2-yl]-1-methyl-pyrazole-4-sulfonamide (approximately 47.80 mg, 0.1000 mmol), potassium carbonate (approximately 23.04 mg, 0.1667 mmol), and tetrakis(triphenylphosphine)palladium(0) (approximately 7.704 mg, 0.006667 mmol) in dioxane (500.0 μL) (0.50 mL) and water (0.05 mL) was microwaved at 120° C. in a sealed vial for 15 minutes. The reaction vials were diluted with DMSO (0.50 mL) and acidified using hydrochloric acid (98.54 μL of 37% w/v, 1.000 mmol). The resultant crude mixture was separated by HPLC (acetonitrile in water with 0.1% hydrochloric acid). N-[4-(2,5-dimethylphenyl)-5-ethyl-6-(4-piperazin-1-ylphenoxy)pyrimidin-2-yl]-1-methyl-pyrazole-4-sulfonamide (2.6 mg, 4.4%). ESI-MS m/z calc. 547.2366, found 548.54 (M+1)⁺; Retention time: 1.4 minutes; LC method A.

### Example 347: Preparation of Compound 1319

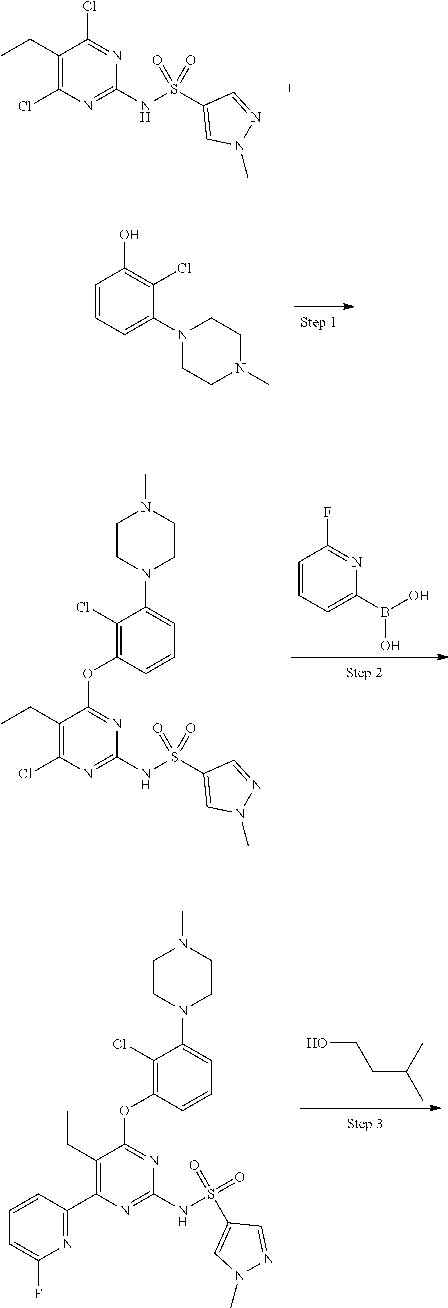

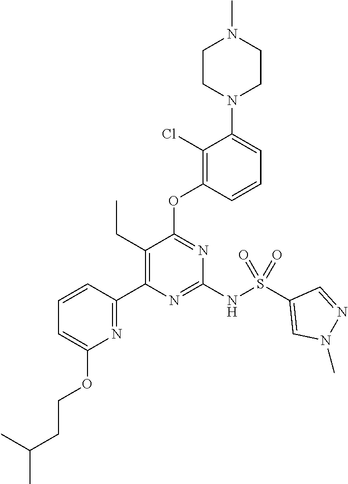 [SMILES: CCc1c(Oc2cccc(N3CCN(C)CC3)c2Cl)nc(NS(=O)(=O)c2cnn(C)c2)nc1-c1cccc(OCCC(C)C)n1]

### Step 1: N-[4-chloro-6-[2-chloro-3-(4-methylpiperazin-1-yl)phenoxy]-5-ethyl-pyrimidin-2-yl]-1-methyl-pyrazole-4-sulfonamide

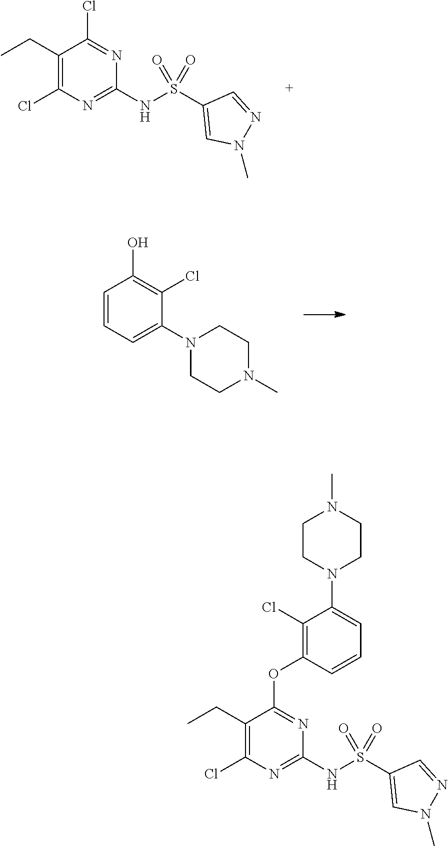 [SMILES: CCc1c(Cl)nc(NS(=O)(=O)c2cnn(C)c2)nc1Cl ; CN1CCN(c2cccc(O)c2Cl)CC1 ; CCc1c(Cl)nc(NS(=O)(=O)c2cnn(C)c2)nc1Oc1cccc(N2CCN(C)CC2)c1Cl]

A heterogeneous solution of N-(4,6-dichloro-5-ethyl-pyrimidin-2-yl)-1-methyl-pyrazole-4-sulfonamide (2.14 g, 6.365 mmol), 2-chloro-3-(4-methylpiperazin-1-yl)phenol (1.44 g, 6.352 mmol), and potassium carbonate (2.64 g, 19.10 mmol) in NMP (6.4 mL) was heated in a sealed vial to 120° C. for 16 hours. The reaction mixture diluted with water (6 mL) and acidified with acetic acid (2.2 mL, 38.69 mmol). The aqueous layer was extracted with dichloromethane (5×), washed with brine (2×), dried over magnesium sulfate, filtered, and concentrated in vacuo. The crude residue was purified by flash column chromatography on silica gel (gradient: 1 to 10% methanol in DCM) to afford N-[4-chloro-6-[2-chloro-3-(4-methylpiperazin-1-yl)phenoxy]-5-ethyl-pyrimidin-2-yl]-1-methyl-pyrazole-4-sulfonamide (2.22g, 62%) as a light yellow solid. ESI-MS m/z calc. 525.11163, found 526.23 (M+1)⁺; Retention time: 0.5 minutes, LC method D.

### Step 2: N-[4-[2-chloro-3-(4-methylpiperazin-1-yl)phenoxy]-5-ethyl-6-(6-fluoro-2-pyridyl)pyrimidin-2-yl]-1-methyl-pyrazole-4-sulfonamide

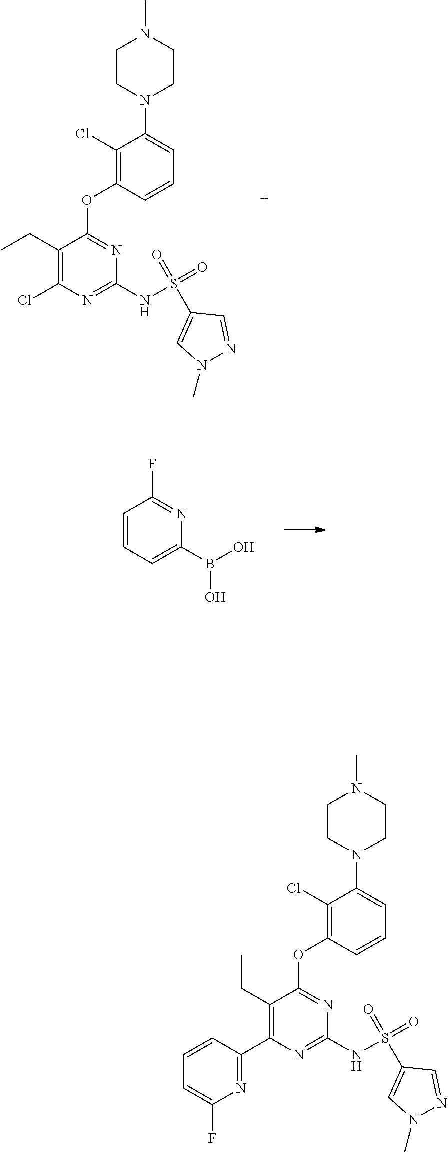 [SMILES: CCc1c(Cl)nc(NS(=O)(=O)c2cnn(C)c2)nc1Oc1cccc(N2CCN(C)CC2)c1Cl ; OB(O)c1cccc(F)n1 ; CCc1c(Oc2cccc(N3CCN(C)CC3)c2Cl)nc(NS(=O)(=O)c2cnn(C)c2)nc1-c1cccc(F)n1]

N-[4-chloro-6-[2-chloro-3-(4-methylpiperazin-1-yl)phenoxy]-5-ethyl-pyrimidin-2-yl]-1-methyl-pyrazole-4-sulfonamide (65.2 mg, 0.1164 mmol), (6-fluoro-2-pyridyl)boronic acid (25.3 mg, 0.1795 mmol), tetrakis(triphenylphosphine)palladium (0) (16 mg, 0.01385 mmol), and 2 M aqueous potassium carbonate (200 μL of 2 M, 0.4000 mmol) were combined in dioxane (1.8 mL) and heated in a sealed vial for 1 hour at 120° C. The reaction was filtered and purified by LC/MS utilizing a gradient of 1-99% acetonitrile in 5 mM aqueous HCl to yield N-[4-[2-chloro-3-(4-methylpiperazin-1-yl)phenoxy]-5-ethyl-6-(6-fluoro-2-pyridyl)pyrimidin-2-yl]-1 -methyl-pyrazole-4-sulfonamide (42.0 mg, 61%) ESI-MS m/z calc. 586.1678, found 587.4 (M+1)⁺; Retention time: 0.52 minutes. LC method D.

### Step 3: N-[4-[2-chloro-3-(4-methylpiperazin-1-yl)phenoxy]-5-ethyl-6-(6-isopentyloxy-2-pyridyl)pyrimidin-2-yl]-1-methyl-pyrazole-4-sulfonamide

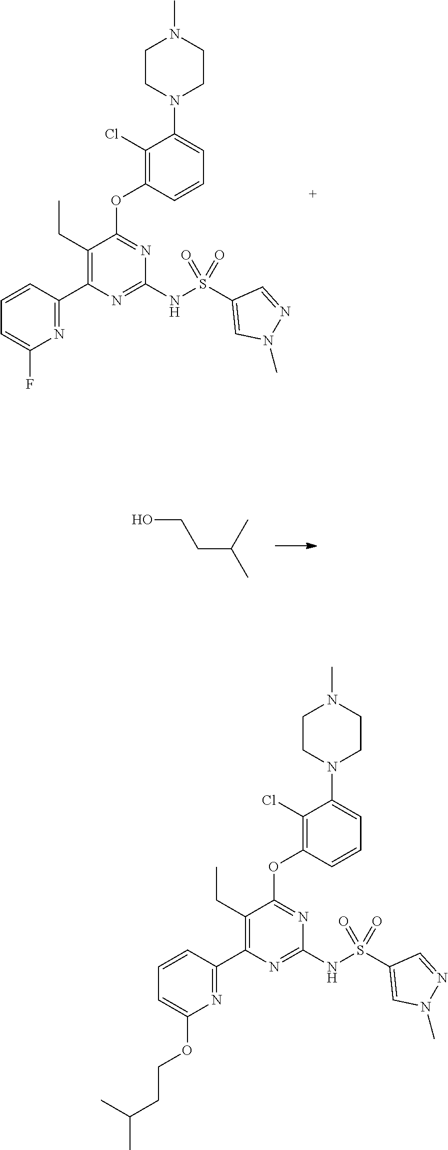

N-[4-[2-chloro-3-(4-methylpiperazin-1-yl)phenoxy]-5-ethyl-6-(6-fluoro-2-pyridyl)pyrimidin-2-yl]-1-methyl-pyrazole-4-sulfonamide (10 mg, 0.01703 mmol) was dissolved in 3-methylbutan-1-ol (500 μL, 4.589 mmol) and cesium carbonate (28.2 mg, 0.08655 mmol) was added. The reaction mixture was heated at 90° C. for 16 hours. The reaction mixture was diluted with DMSO (0.5 mL), filtered, and purified by LC/MS utilizing a gradient of 1-99% acetonitrile in 5 mM aqueous HCl to yield N-[4-[2-chloro-3-(4-methylpiperazin-1-yl)phenoxy]-5-ethyl-6-(6-isopentyloxy-2-pyridyl)pyrimidin-2-yl]-1-methyl-pyrazole-4-sulfonamide (hydrochloride salt) (4.4 mg, 37%) ESI-MS m/z calc. 654.25037, found 655.5 (M+1)⁺; Retention time: 1.78 minutes. LC method A.

### Example 348: Preparation of Compound 1320

### Step 1: N-[4-[2-chloro-3-(4-methylpiperazin-1-yl)phenoxy]-5-ethyl-6-(2-isopropoxy-3-pyridyl)pyrimidin-2-yl]-1-methyl-pyrazole-4-sulfonamide

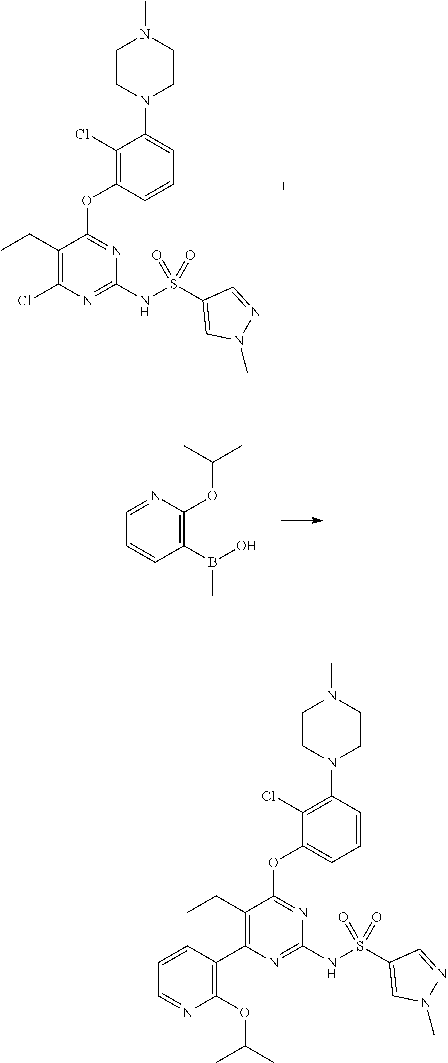 [SMILES: CCc1c(Cl)nc(NS(=O)(=O)c2cnn(C)c2)nc1Oc1cccc(N2CCN(C)CC2)c1Cl ; CB(O)c1cccnc1OC(C)C ; CCc1c(Oc2cccc(N3CCN(C)CC3)c2Cl)nc(NS(=O)(=O)c2cnn(C)c2)nc1-c1cccnc1OC(C)C]

N-[4-chloro-6-[2-chloro-3-(4-methylpiperazin-1-yl)phenoxy]-5-ethyl-pyrimidin-2-yl]-1-methyl-pyrazole-4-sulfonamide (30 mg, 0.05357 mmol), (2-isopropoxy-3-pyridyl)boronic acid (approximately 11.63 mg, 0.06428 mmol), tetrakis(triphenylphosphine)palladium (0) (approximately 6.190 mg, 0.005357 mmol), and 2 M aqueous potassium carbonate (approximately 107.2 μL of 2 M, 0.2143 mmol) were combined in dioxane (0.9 mL) and irradiated in the microwave for 30 minutes at 120° C. The reaction mixture was filtered and purified by LC/MS utilizing a gradient of 1-99% acetonitrile in 5 mM aqueous HCl to yield N-[4-[2-chloro-3-(4-methylpiperazin-1-yl)phenoxy]-5-ethyl-6-(2-isopropoxy-3-pyridyl)pyrimidin-2-yl]-1-methyl-pyrazole-4-sulfonamide (hydrochloride salt) (3.8 mg, 10%). ESI-MS m/z calc. 626.21906, found 627.27 (M+1)⁺; Retention time: 1.02 minutes; LC method A.

### Example 349: Preparation of Compound 1321

### Step 1: N-[5-ethyl-4,6-bis(o-tolyl)pyrimidin-2-yl]-1-methyl-pyrazole-4-sulfonamide

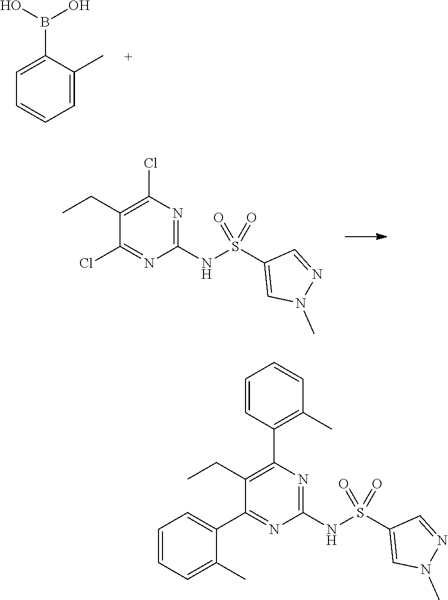

A mixture of N-(4,6-dichloro-5-ethyl-pyrimidin-2-yl)-1-methyl-pyrazole-4-sulfonamide (200 mg, 0.5949 mmol), o-tolylboronic acid (89.7 mg, 0.6598 mmol), Pd(dppf)Cl₂ (45.1 mg, 0.06164 mmol), and potassium carbonate (629 μL of 2 M, 1.258 mmol) in 1,2-dimethoxyethane (1.3 mL) was degassed by flow of nitrogen and the reaction was stirred at 130° C. for 10 minutes in a pressure vessel. The reaction was filtered. EtOAc and water were added to the reaction and acidified with 1 N HCl. The two layers were separated, and the aqueous layer was extracted with EtOAc (×2). The combined organic layer was dried over Na₂SO₄, filtered and concentrated. The crude material was purified on 40 g of silica gel utilizing a gradient of 0-100% ethyl acetate in hexane to yield N-[5-ethyl-4,6-bis(o-tolyl)pyrimidin-2-yl]-1-methyl-pyrazole-4-sulfonamide (43 mg, 16%). ¹H NMR (400 MHz, DMSO-d₆) δ 11.57 (s, 1H), 8.08 (s, 1H), 7.62 (s, 1H), 7.41 (dd, J=7.7, 1.5 Hz, 1H), 7.39-7.35 (m, 3H), 7.33 (td, J=6.8, 6.3, 2.4 Hz, 2H), 7.27 (dd, J=7.4, 1.3 Hz, 2H), 3.77 (s, 3H), 2.21-1.93 (m, 8H), 0.55 (t, J=7.4 Hz, 3H). ESI-MS m/z calc. 447.17288, found 448.2 (M+1)⁺; Retention time: 1.89 minutes (LC method A).

### Example 350: Preparation of Compound 1322

### Step 1: N-(5-Ethyl-4,6-diphenyl-pyrimidin-2-yl)benzenesulfonamide

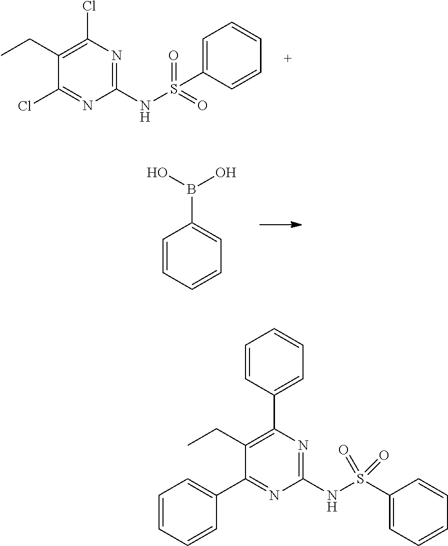 [SMILES: CCc1c(Cl)nc(NS(=O)(=O)c2ccccc2)nc1Cl ; OB(O)c1ccccc1 ; CCc1c(-c2ccccc2)nc(NS(=O)(=O)c2ccccc2)nc1-c1ccccc1]

A mixture of N-(4,6-dichloro-5-ethyl-pyrimidin-2-yl)benzenesulfonamide (157 mg, 0.4726 mmol), phenylboronic acid (90 mg, 0.7381 mmol), Pd(dppf)C₁₂ (59 mg, 0.08063 mmol), dioxane (4.2 mL) and potassium carbonate (494 μL of 2 M, 0.9880 mmol) was degassed by flow of nitrogen and the reaction was stirred at 110° C. for 1 hour and 15 minutes in a pressure vessel. The cooled mixture was filtered and concentrated in vacuo. The crude product was dissolved in DMSO, filtered and purified using a reverse phase HPLC C₁₈ column and a dual gradient run from 1-99% mobile phase B over 30 minutes (Mobile phase A=H₂O (5 mM HCl). Mobile phase B═CH₃CN) to yield N-(5-ethyl-4,6-diphenyl-pyrimidin-2-yl)benzenesulfonamide (97.4 mg, 50%) as a brown solid. ¹H NMR (400 MHz, DMSO-d₆) δ 11.78 (s, 1H), 7.93 (d, J=7.9 Hz, 2H), 7.66 (t, J=7.3 Hz, 1H), 7.59-7.49 (m, 8H), 7.44 (dd, J=6.7, 3.0 Hz, 4H), 2.60 (q, J=7.5 Hz, 2H), 0.59 (t, J=7.4 Hz, 3H). ESI-MS m/z calc. 415.13544, found 416.3 (M+1)⁺; Retention time: 1.97 minutes (LC method A).

### Example 351: Preparation of Compound 1323

### Step 1: N-[4-(2,3-Dimethylphenyl)-5-ethyl-6-(4-piperazin-1-ylphenoxy)pyrimidin-2-yl]-1-methyl-pyrazole-4-sulfonamide

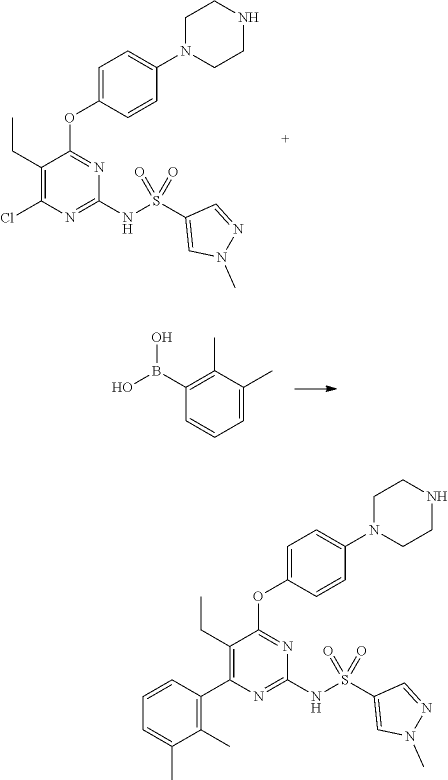 [SMILES: CCc1c(Cl)nc(NS(=O)(=O)c2cnn(C)c2)nc1Oc1ccc(N2CCNCC2)cc1 ; Cc1cccc(B(O)O)c1C ; CCc1c(Oc2ccc(N3CCNCC3)cc2)nc(NS(=O)(=O)c2cnn(C)c2)nc1-c1cccc(C)c1C]

A heterogeneous mixture of (2,3-dimethylphenyl)boronic acid (approximately 44.99 mg, 0.3000 mmol), N-[4-chloro-5-ethyl-6-(4-piperazin-1-ylphenoxy)pyrimidin-2-yl]-1-methyl-pyrazole-4-sulfonamide (approximately 47.80 mg, 0.1000 mmol), potassium carbonate (approximately 23.04 mg, 0.1667 mmol), and tetrakis(triphenylphosphine)palladium(0) (approximately 7.704 mg, 0.006667 mmol) in dioxane (500.0 μL) (0.50 mL) and water (0.05 mL) was microwaved at 120° C. in a sealed vial for 15 minutes. The reaction vials were diluted with DMSO (0.50 mL) and acidified using hydrochloric acid (98.54 μL of 37% w/v, 1.000 mmol). The resultant crude mixture was separated by HPLC (acetonitrile in water with 0.1% hydrochloric acid). N-[4-(2,3-dimethylphenyl)-5-ethyl-6-(4-piperazin-1-ylphenoxy)pyrimidin-2-yl]-1-methyl-pyrazole-4-sulfonamide (4.4 mg, 8%). ESI-MS m/z calc. 547.2366, found 548.54 (M+1)⁺; Retention time: 1.37 minutes; LC method A.

### Example 352: Preparation of Compound 1324

### Step 1: N-[5-ethyl-4-(4-piperazin-1-ylphenoxy)-6-(2,3,5,6-tetramethylphenyl)pyrimidin-2-yl]-1-methyl-pyrazole-4-sulfonamide

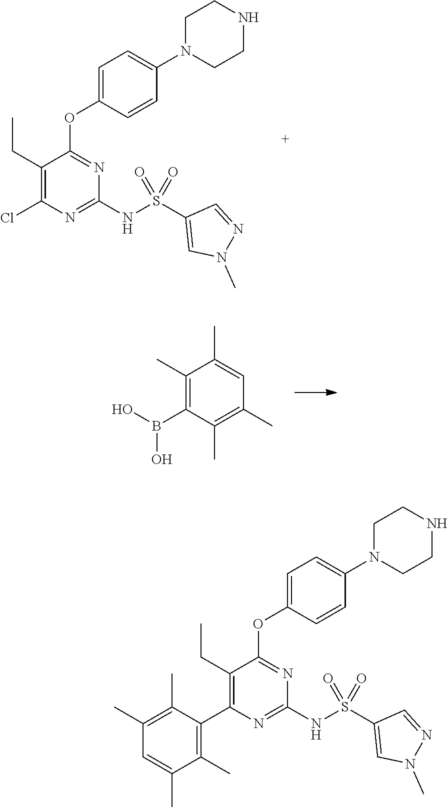

The compound was prepared in a manner analogous to that described above using commercially available (2,3,5,6-tetramethylphenyl)boronic acid to give N-[5-ethyl-4-(4-piperazin-1-ylphenoxy)-6-(2,3,5,6-tetramethylphenyl)pyrimidin-2-yl]-1-methyl-pyrazole-4-sulfonamide (2.6 mg, 4%). ESI-MS m/z calc. 575.2679, found 576.55 (M+1)⁺; Retention time: 1.5 minutes; LC method A.

### Example 353: Preparation of Compound 1325

### Step 1: N-[5-ethyl-4-(2-isobutylphenyl)-6-(4-piperazin-1-ylphenoxy)pyrimidin-2-yl]-1-methyl-pyrazole-4-sulfonamide

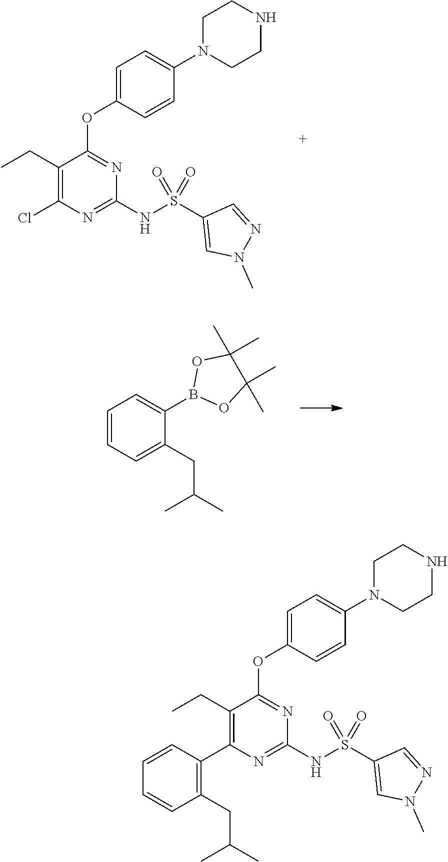 [SMILES: CCc1c(Cl)nc(NS(=O)(=O)c2cnn(C)c2)nc1Oc1ccc(N2CCNCC2)cc1 ; CC(C)Cc1ccccc1B1OC(C)(C)C(C)(C)O1 ; CCc1c(Oc2ccc(N3CCNCC3)cc2)nc(NS(=O)(=O)c2cnn(C)c2)nc1-c1ccccc1CC(C)C]

The compound was prepared in a manner analogous to that described above using commercially available 2-(2-isobutylphenyl)-4,4,5,5-tetramethyl-1,3,2-dioxaborolane to give N-[5-ethyl-4-(2-isobutylphenyl)-6-(4-piperazin-1-ylphenoxy)pyrimidin-2-yl]-1-methyl-pyrazole-4-sulfonamide (1 mg, 2%) was furnished as a yellow solid. ESI-MS m/z calc. 575.2679, found 576.66 (M+1)⁺; Retention time. 1.54 minutes, LC method A.

### Example 354: Preparation of Compound 1326

### Step 1: N-[5-ethyl-4-(2-isopropylphenyl)-6-(4-piperazin-1-ylphenoxy)pyrimidin-2-yl]-1-methyl-pyrazole-4-sulfonamide

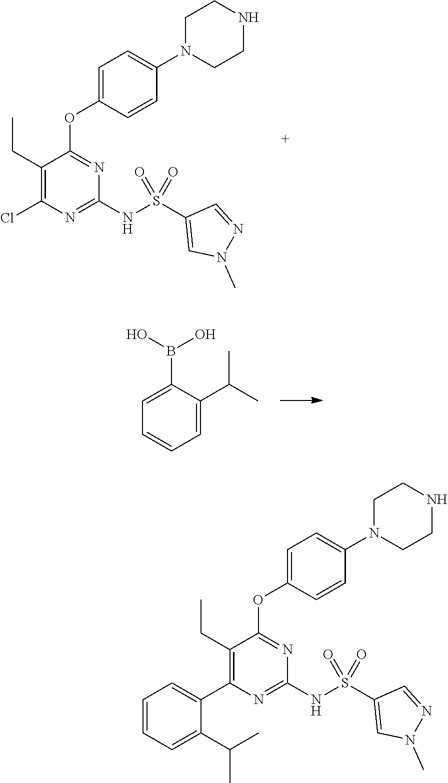 [SMILES: CCc1c(Cl)nc(NS(=O)(=O)c2cnn(C)c2)nc1Oc1ccc(N2CCNCC2)cc1 ; CC(C)c1ccccc1B(O)O ; CCc1c(Oc2ccc(N3CCNCC3)cc2)nc(NS(=O)(=O)c2cnn(C)c2)nc1-c1ccccc1C(C)C]

The compound was prepared in a manner analogous to that described above using commercially available (2-isopropylphenyl)boronic acid to give N-[5-ethyl-4-(2-isopropylphenyl)-6-(4-piperazin-1-ylphenoxy)pyrimidin-2-yl]-1-methyl-pyrazole-4-sulfonamide (6.5 mg, 11%). ESI-MS m/z calc. 561.2522, found 562.55 (M+1)⁺; Retention time: 1.46 minutes; LC method A.

### Example 355: Preparation of Compound 1327

### Step 1: N-[4-(2-chlorophenyl)-5-ethyl-6-(4-piperazin-1-ylphenoxy)pyrimidin-2-yl]-1-methyl-pyrazole-4-sulfonamide

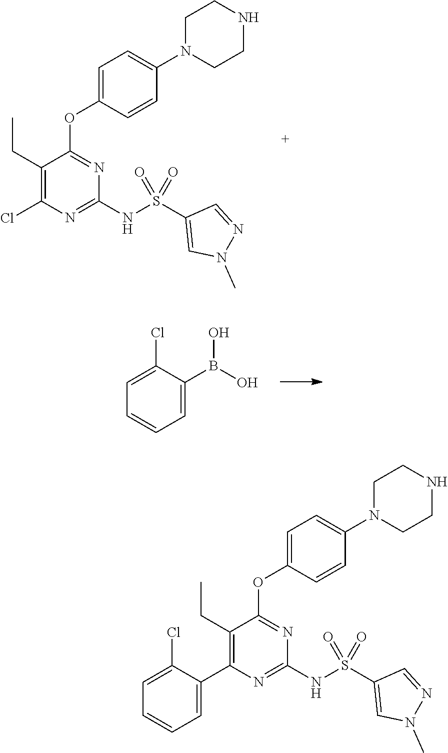 [SMILES: CCc1c(Cl)nc(NS(=O)(=O)c2cnn(C)c2)nc1Oc1ccc(N2CCNCC2)cc1 ; OB(O)c1ccccc1Cl ; CCc1c(Oc2ccc(N3CCNCC3)cc2)nc(NS(=O)(=O)c2cnn(C)c2)nc1-c1ccccc1Cl]

The compound was prepared in a manner analogous to that described above using commercially available (2-chlorophenyl)boronic acid to give N-[4-(2-chlorophenyl)-5-ethyl-6-(4-piperazin-1-ylphenoxy)pyrimidin-2-yl]-1-methyl-pyrazole-4-sulfonamide (1.7 mg, 3%). ESI-MS m/z calc. 553.16626, found 554.46 (M+1)⁺; Retention time: 1.33 minutes; LC method A.

### Example 356: Preparation of Compound 1328

### Step 1: N-[4-(2-cyclobutylphenyl)-5-ethyl-6-(4-piperazin-1-ylphenoxy)pyrimidin-2-yl]-1-methyl-pyrazole-4-sulfonamide

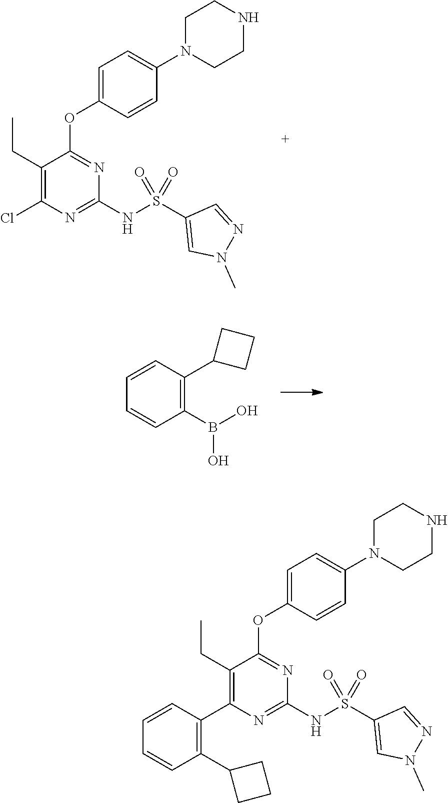

The compound was prepared in a manner analogous to that described above using commercially available (2-cyclobutylphenyl)boronic acid to give N-[4-(2-cyclobutylphenyl)-5-ethyl-6-(4-piperazin-1-ylphenoxy)pyrimidin-2-yl]-1-methyl-pyrazole-4-sulfonamide (3.9 mg, 6%). ESI-MS m/z calc. 573.2522, found 574.56 (M+1)⁺; Retention time: 1.5 minutes; LC method A.

### Example 357: Preparation of Compound 1329

### Step 1: N-[5-ethyl-4-(4-piperazin-1-ylphenoxy)-6-(2,4,6-trimethylphenyl)pyrimidin-2-yl]-1-methyl-pyrazole-4-sulfonamide

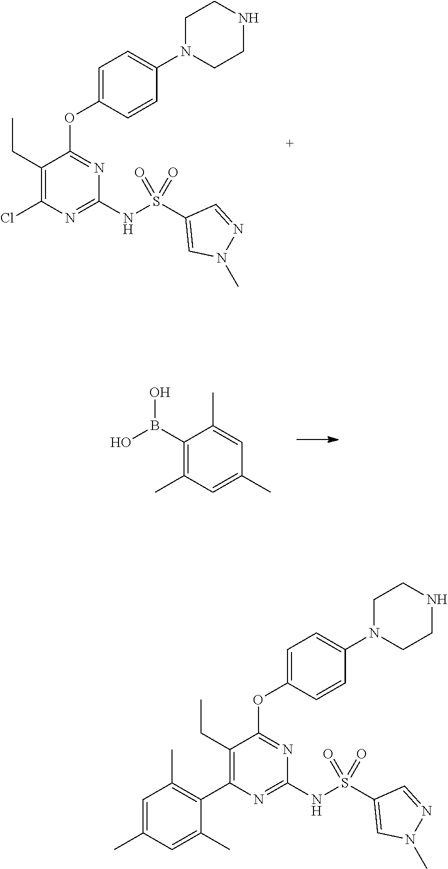 [SMILES: CCc1c(Cl)nc(NS(=O)(=O)c2cnn(C)c2)nc1Oc1ccc(N2CCNCC2)cc1 ; Cc1cc(C)c(B(O)O)c(C)c1 ; CCc1c(Oc2ccc(N3CCNCC3)cc2)nc(NS(=O)(=O)c2cnn(C)c2)nc1-c1c(C)cc(C)cc1C]

The compound was prepared in a manner analogous to that described above using commercially available (2,4,6-trimethylphenyl)boronic acid to give N-[5-ethyl-4-(4-piperazin-1-ylphenoxy)-6-(2,4,6-trimethylphenyl)pyrimidin-2-yl]-1-methyl-pyrazole-4-sulfonamide (3 mg, 5%). ESI-MS m/z calc. 561.2522, found 562.34 (M+1)⁺; Retention time: 1.03 minutes; LC method E.

### Example 358: Preparation of Compound 1340

### Step 1: N-[4-[2-chloro-3-(4-methylpiperazin-1-yl)phenoxy]-5-ethyl-6-(3-isopentyloxy-2,6-dimethyl-phenyl)pyrimidin-2-yl]-1-methyl-pyrazole-4-sulfonamide

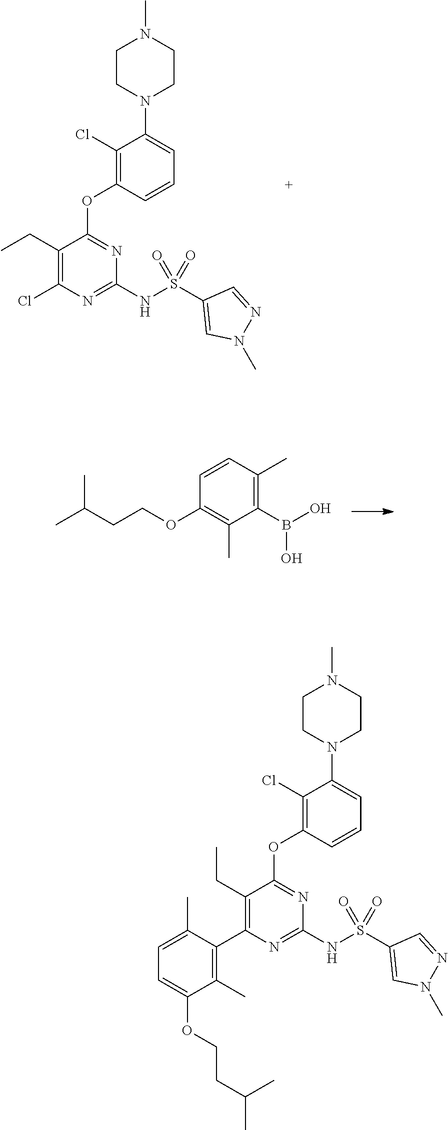 [SMILES: CCc1c(Cl)nc(NS(=O)(=O)c2cnn(C)c2)nc1Oc1cccc(N2CCN(C)CC2)c1Cl ; Cc1ccc(OCCC(C)C)c(C)c1B(O)O ; CCc1c(Oc2cccc(N3CCN(C)CC3)c2Cl)nc(NS(=O)(=O)c2cnn(C)c2)nc1-c1c(C)ccc(OCCC(C)C)c1C]

A heterogeneous solution of (3-isopentyloxy-2,6-dimethyl-phenyl)boronic acid (27.4 mg, 0.1160 mmol), potassium carbonate (48.1 mg, 0.3480 mmol), tetrakis(triphenylphosphine)palladium(0) (26.8 mg, 0.02319 mmol), and N-[4-chloro-6-[2-chloro-3-(4-methylpiperazin-1-yl)phenoxy]-5-ethyl-pyrimidin-2-yl]-1-methyl-pyrazole-4-sulfonamide (65.0 mg, 0.1161 mmol) in dioxane (400 μL) and water (80 μL) was microwaved in a sealed vial to 130° C. for 3 hours. The reaction mixture was acidified with acetic acid (100 μL, 1.758 mmol), diluted with DMSO (0.5 mL), and filtered through a 0.45 μM PTFE syringe filter. The sample was purified by reverse phase HPLC (Phenomenex Luna C₁₈ column (75×30 mm, m particle size), gradient: 1-99% acetonitrile in water (5 mM HCl) over 15.0 minutes) to afford N-[4-[2-chloro-3-(4-methylpiperazin-1-yl)phenoxy]-5-ethyl-6-(3-isopentyloxy-2,6-dimethyl-phenyl)pyrimidin-2-yl]-1-methyl-pyrazole-4-sulfonamide (hydrochloride salt) (1.8 mg, 2%) as a yellow oil. ESI-MS m/z calc. 681.2864, found 682.41 (M+1)⁺; Retention time: 1.75 minutes. LC method A.

### Example 359: Preparation of Compound 1341

### Step 1: N-[4-[2-chloro-3-(4-methylpiperazin-1-yl)phenoxy]-5-ethyl-6-(2-isopentyphenyl)pyrimidin-2-yl]-1-methyl-pyrazole-4-sulfonamide

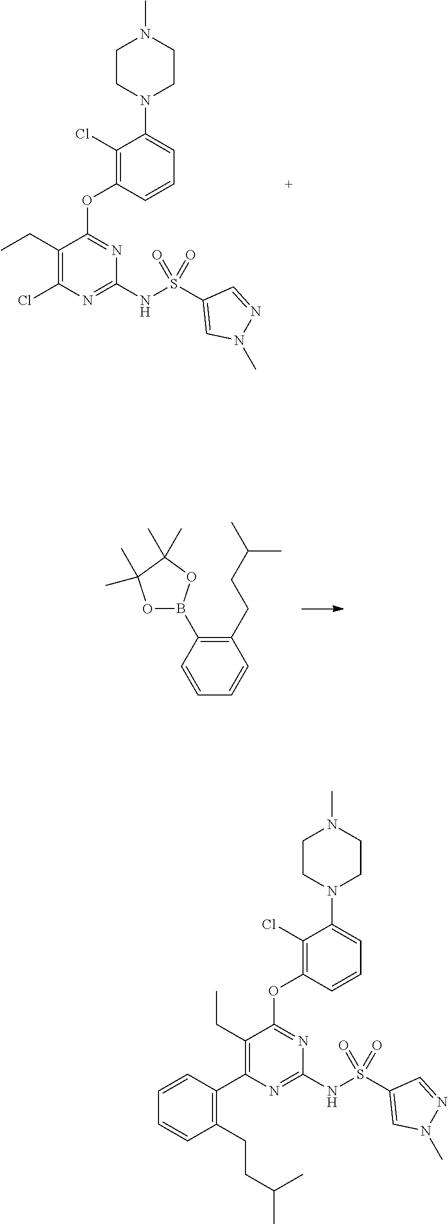 [SMILES: CCc1c(Cl)nc(NS(=O)(=O)c2cnn(C)c2)nc1Oc1cccc(N2CCN(C)CC2)c1Cl ; CC(C)CCc1ccccc1B1OC(C)(C)C(C)(C)O1 ; CCc1c(Oc2cccc(N3CCN(C)CC3)c2Cl)nc(NS(=O)(=O)c2cnn(C)c2)nc1-c1ccccc1CCC(C)C]

N-[4-chloro-6-[2-chloro-3-(4-methylpiperazin-1-yl)phenoxy]-5-ethyl-pyrimidin-2-yl]-1-methyl-pyrazole-4-sulfonamide (19.2 mg, 0.03428 mmol), 2-(2-isopentylphenyl)-4,4,5,5-tetramethyl-1,3,2-dioxaborolane (17 mg, 0.06200 mmol), tetrakis(triphenylphosphine)palladium (0) (6.1 mg, 0.005279 mmol), and 2 M aqueous potassium carbonate (100 μL of 2 M, 0.2000 mmol) were combined in dioxane (900 μL) and heated in a sealed vial for 1 hours at 120° C. The reaction was filtered and purified by LC/MS utilizing a gradient of 1-99% acetonitrile in 5 mM aqueous HCl to yield N-[4-[2-chloro-3-(4-methylpiperazin-1-yl)phenoxy]-5-ethyl-6-(2-isopentylphenyl)pyrimidin-2-yl]-1-methyl-pyrazole-4-sulfonamide (hydrochloride salt) (6.8 mg, 29%) ESI-MS m/z calc. 637.2602, found 638.5 (M+1)⁺; Retention time: 1.64 minutes. LC method A.

### Example 360: Preparation of Compound 1342

### Step 1: N-[4-[2-chloro-3-(4-methylpiperazin-1-yl)phenoxy]-5-ethyl-6-(2-isobutylphenyl)pyrimidin-2-yl]-1-methyl-pyrazole-4-sulfonamide

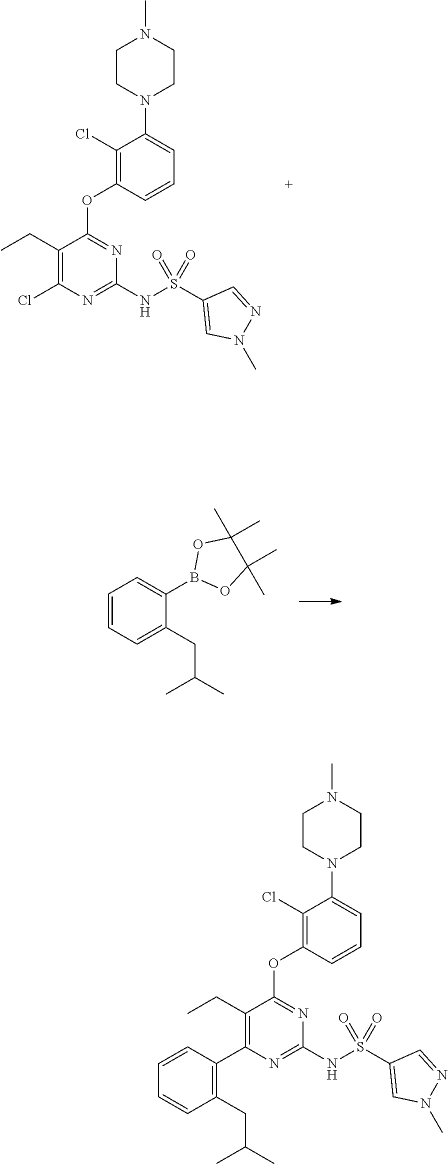 [SMILES: CCc1c(Cl)nc(NS(=O)(=O)c2cnn(C)c2)nc1Oc1cccc(N2CCN(C)CC2)c1Cl ; CC(C)Cc1ccccc1B1OC(C)(C)C(C)(C)O1 ; CCc1c(Oc2cccc(N3CCN(C)CC3)c2Cl)nc(NS(=O)(=O)c2cnn(C)c2)nc1-c1ccccc1CC(C)C]

A dioxane (0.5 mL) mixture of N-[4-chloro-6-[2-chloro-3-(4-methylpiperazin-1-yl)phenoxy]-5-ethyl-pyrimidin-2-yl]-1-methyl-pyrazole-4-sulfonamide (hydrochloride salt) (19.7 mg, 0.03500 mmol), 2-(2-isobutylphenyl)-4,4,5,5-tetramethyl-1,3,2-dioxaborolane (9.244 mg, 0.03553 mmol), Pd(dppf)Cl₂(2.902 mg, 0.003554 mmol), and K₂CO₃ (71.05 μL of 2 M, 0.1421 mmol) was sparged under nitrogen for 2 minutes and then microwaved at 120° C. for 30 minutes. The solution was filtered and the filtrate dissolved in 0.8 mL DMSO, and purified by reverse phase chromatography using a 15 minute gradient of 20% MeCN in water to 80% MeCN (HCl modifier) to give N-[4-[2-chloro-3-(4-methylpiperazin-1-yl)phenoxy]-5-ethyl-6-(2-isobutylphenyl)pyrimidin-2-yl]-1-methyl-pyrazole-4-sulfonamide (hydrochloride salt) (1.5 mg, 6%) ESI-MS m/z calc. 623.2445, found 624.3 (M+1)⁺; Retention time: 1.58 minutes. LC method A.

### Example 361: Preparation of Compound 1343

### Step 1: N-[4-[2-chloro-3-(4-methylpiperazin-1-yl)phenoxy]-5-ethyl-6-(2-isopropylphenyl)pyrimidin-2-yl]-1-methyl-pyrazole-4-sulfonamide

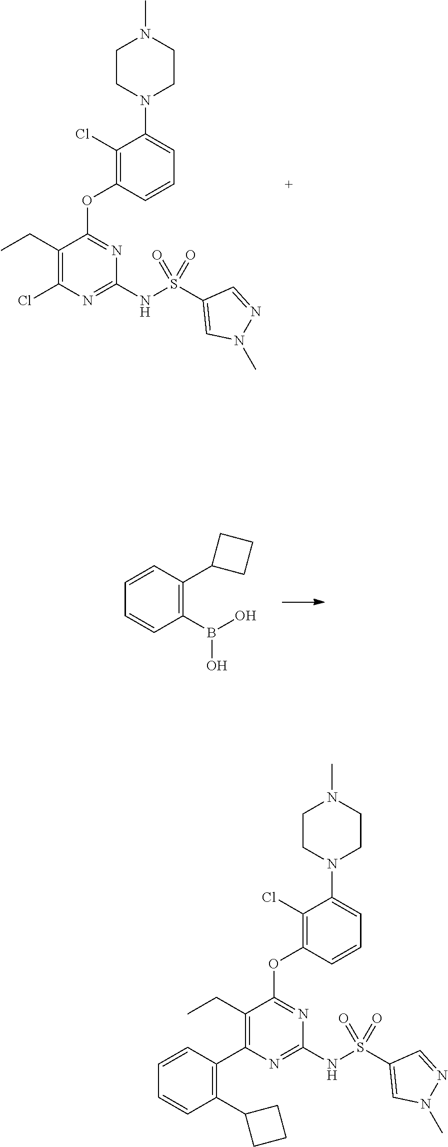 [SMILES: CCc1c(Cl)nc(NS(=O)(=O)c2cnn(C)c2)nc1Oc1cccc(N2CCN(C)CC2)c1Cl ; OB(O)c1ccccc1C1CCC1 ; CCc1c(Oc2cccc(N3CCN(C)CC3)c2Cl)nc(NS(=O)(=O)c2cnn(C)c2)nc1-c1ccccc1C1CCC1]

The compound was prepared in a manner analogous to that described above using commercially available (2-cyclobutylphenyl)boronic acid to give N-[4-[2-chloro-3-(4-methylpiperazin-1-yl)phenoxy]-6-(2-cyclobutylphenyl)-5-ethyl-pyrimidin-2-yl]-1-methyl-pyrazole-4-sulfonamide (hydrochloride salt) (1.0 mg, 4%) ESI-MS m/z calc. 621.2289, found 622.2 (M+1)⁺; Retention time: 1.52 minutes. LC method A.

### Example 362: Preparation of Compound 1344

### Step 1:N-[4-[2-chloro-3-(4-methylpiperazin-1-yl)phenoxy]-5-ethyl-6-(2-isopropylphenyl)pyrimidin-2-yl]-1-methyl-pyrazole-4-sulfonamide

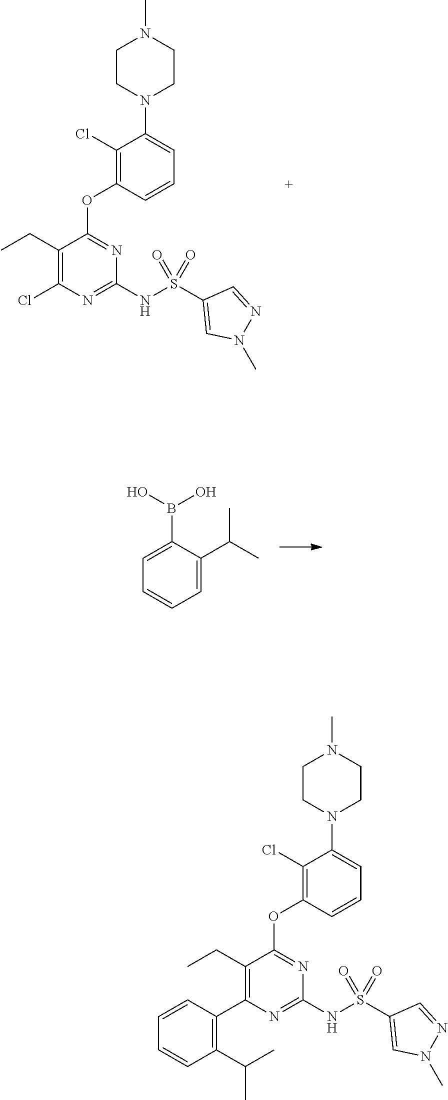 [SMILES: CCc1c(Cl)nc(NS(=O)(=O)c2cnn(C)c2)nc1Oc1cccc(N2CCN(C)CC2)c1Cl ; CC(C)c1ccccc1B(O)O ; CCc1c(Oc2cccc(N3CCN(C)CC3)c2Cl)nc(NS(=O)(=O)c2cnn(C)c2)nc1-c1ccccc1C(C)C]

The compound was prepared in a manner analogous to that described above using commercially available (2-isopropylphenyl)boronic acid to give N-[4-[2-chloro-3-(4-methylpiperazin-1-yl)phenoxy]-5-ethyl-6-(2-isopropylphenyl)pyrimidin-2-yl]-1-methyl-pyrazole-4-sulfonamide (hydrochloride salt) (2.4 mg, 11%) ESI-MS m/z calc. 609.2289, found 610.2 (M+1)⁺; Retention time: 1.51 minutes. LC method A.

### Example 363: Preparation of Compound 1345

### Step 1: N-[4-[2-chloro-3-(4-methylpiperazin-1-yl)phenoxy]-5-ethyl-6-(3-isopropoxyphenyl)pyrimidin-2-yl]-1-methyl-pyrazole-4-sulfonamide

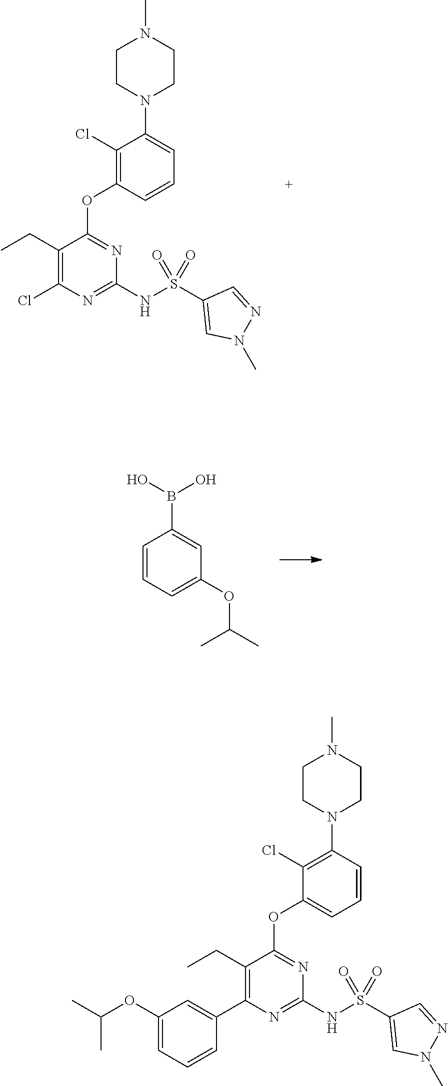 [SMILES: CCc1c(Cl)nc(NS(=O)(=O)c2cnn(C)c2)nc1Oc1cccc(N2CCN(C)CC2)c1Cl ; CC(C)Oc1cccc(B(O)O)c1 ; CCc1c(Oc2cccc(N3CCN(C)CC3)c2Cl)nc(NS(=O)(=O)c2cnn(C)c2)nc1-c1cccc(OC(C)C)c1]

N-[4-chloro-6-[2-chloro-3-(4-methylpiperazin-1-yl)phenoxy]-5-ethyl-pyrimidin-2-yl]-1-methyl-pyrazole-4-sulfonamide (30 mg, 0.05357 mmol), (3-isopropoxyphenyl)boronic acid (approximately 11.57 mg, 0.06428 mmol), tetrakis(triphenylphosphine)palladium (0) (approximately 6.190 mg, 0.005357 mmol), and 2 M aqueous potassium carbonate (approximately 107.2 μL of 2 M, 0.2143 mmol) were combined in dioxane (0.9 mL) and irradiated in the microwave for 30 minutes at 120° C. The reaction was filtered and purified by LC/MS utilizing a gradient of 1-99% acetonitrile in 5 mM aqueous HCl to yield N-[4-[2-chloro-3-(4-methylpiperazin-1-yl)phenoxy]-5-ethyl-6-(3-isopropoxyphenyl)pyrimidin-2-yl]-1-methyl-pyrazole-4-sulfonamide (hydrochloride salt) (7.3 mg, 20%). ESI-MS m/z calc. 625.2238, found 626.4 (M+1)⁺; Retention time: 1.4 minutes; LC method A.

### Example 364: Preparation of Compound 1346

### Step 1: N-[4-[2-chloro-3-(4-methylpiperazin-1-yl)phenoxy]-5-ethyl-6-(o-tolyl)pyrimidin-2-yl]-1-methyl-pyrazole-4-sulfonamide

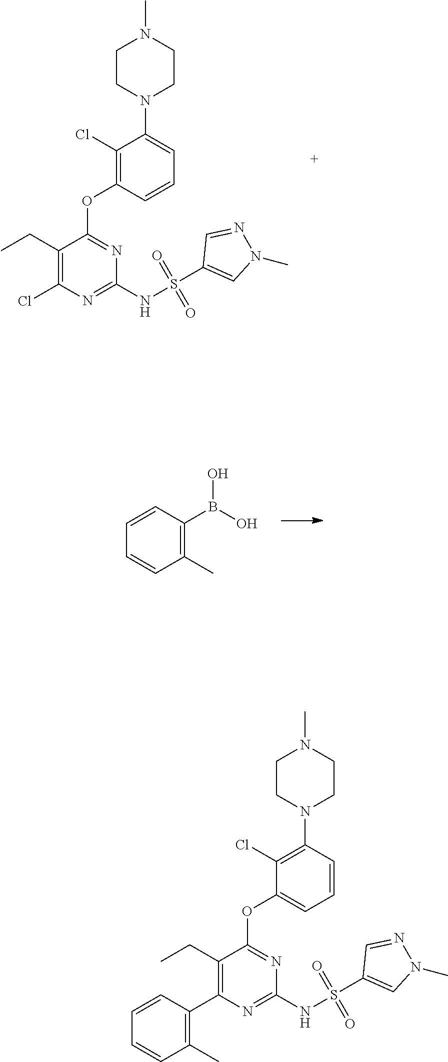

N-[4-chloro-6-[2-chloro-3-(4-methylpiperazin-1-yl)phenoxy]-5-ethyl-pyrimidin-2-yl]-1-methyl-pyrazole-4-sulfonamide (30 mg, 0.05699 mmol) and o-tolylboronic acid (10 mg, 0.07355 mmol) were combined and dissolved in dioxane (1 mL). Aqueous potassium carbonate (115 μL of 2 M, 0.2300 mmol) was added. tetrakis(triphenylphosphine)palladium (0) (4 mg, 0.003462 mmol) was added under nitrogen gas. The reaction vessel was sealed and heated under microwave irradiation at 120° C. for 20 minutes. The product was isolated by UV-triggered reverse-phase HPLC: Samples were purified using a reverse phase HPLC method using a Luna Cis (2) column (50×21.2 mm, 5 μm particle size) sold by Phenomenex (pn: 00B-4252-PO-AX), and a dual gradient run from 10-70% mobile phase B over 15.0 minutes. Mobile phase A=water (5 mM HCl acid modifier). Mobile phase B=acetonitrile. Flow rate=35 mL/min, injection volume=950 μL, and column temperature=25° C. The UV trace at 220 nm was used to collect fractions. N-[4-[2-chloro-3-(4-methylpiperazin-1-yl)phenoxy]-5-ethyl-6-(o-tolyl)pyrimidin-2-yl]-1-methyl-pyrazole-4-sulfonamide (hydrochloride salt) (5.3 mg, 15%) was obtained. ESI-MS m/z calc. 581.1976, found 582.4 (M+1)⁺; Retention time: 1.35 minutes (LC method A).

### Example 365: Preparation of Compound 1347

### Step 1: N-[4-(2-cyclobutylphenyl)-5-ethyl-6-[2-fluoro-3-(4-methylpiperazin-1-yl)phenoxy]pyramidin-2-yl]-1-methyl-pyrazole-4-sulfonamide

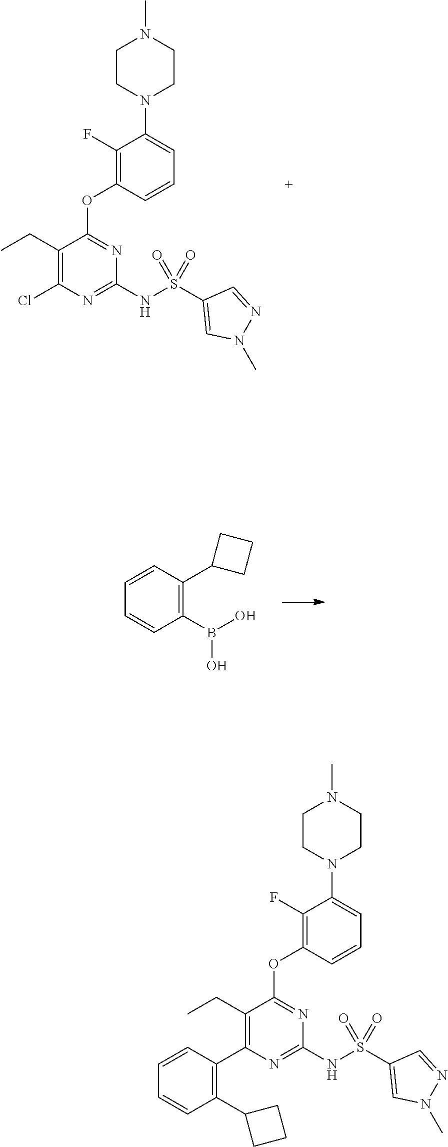 [SMILES: CCc1c(Cl)nc(NS(=O)(=O)c2cnn(C)c2)nc1Oc1cccc(N2CCN(C)CC2)c1F ; OB(O)c1ccccc1C1CCC1 ; CCc1c(Oc2cccc(N3CCN(C)CC3)c2F)nc(NS(=O)(=O)c2cnn(C)c2)nc1-c1ccccc1C1CCC1]

The compound was prepared in a manner analogous to that described above using commercially available (2-cyclobutylphenyl)boronic acid to give N-[4-(2-cyclobutylphenyl)-5-ethyl-6-[2-fluoro-3-(4-methylpiperazin-1-yl)phenoxy]pyrimidin-2-yl]-1-methyl-pyrazole-4-sulfonamide (hydrochloride salt) (3.4 mg, 14%) ESI-MS m/z calc. 605.2584, found 606.32 (M+1)⁺; Retention time: 1.52 minutes. LC method A.

### Example 366: Preparation of Compound 1348

### Step 1: N-[4-[2-chloro-3-(4-methylpiperazin-1-yl)phenoxy]-5-ethyl-6-(2-isobutoxyphenyl)pyrimidin-2-yl]-1-methyl-pyrazole-4-sulfonamide

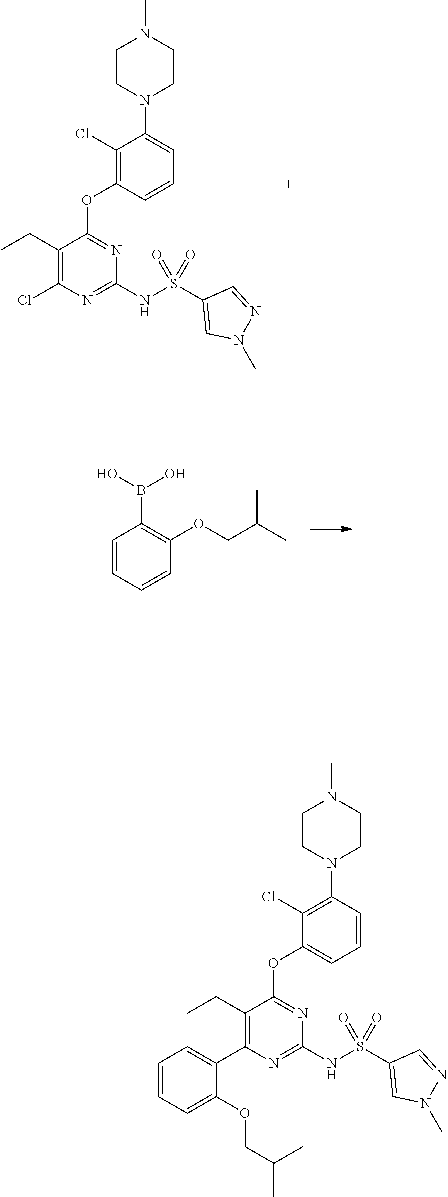 [SMILES: CCc1c(Cl)nc(NS(=O)(=O)c2cnn(C)c2)nc1Oc1cccc(N2CCN(C)CC2)c1Cl ; CC(C)COc1ccccc1B(O)O ; CCc1c(Oc2cccc(N3CCN(C)CC3)c2Cl)nc(NS(=O)(=O)c2cnn(C)c2)nc1-c1ccccc1OCC(C)C]

The compound was prepared in a manner analogous to that described above using commercially available (2-isobutoxyphenyl)boronic acid to give N-[4-[2-chloro-3-(4-methylpiperazin-1-yl)phenoxy]-5-ethyl-6-(2-isobutoxyphenyl)pyrimidin-2-yl]-1-methyl-pyrazole-4-sulfonamide (hydrochloride salt) (5.8 mg, 16%). ESI-MS m/z calc. 639.23944, found 640.4 (M+1)⁺; Retention time: 1.41 minutes; LC method A.

### Example 367: Preparation of Compound 1349

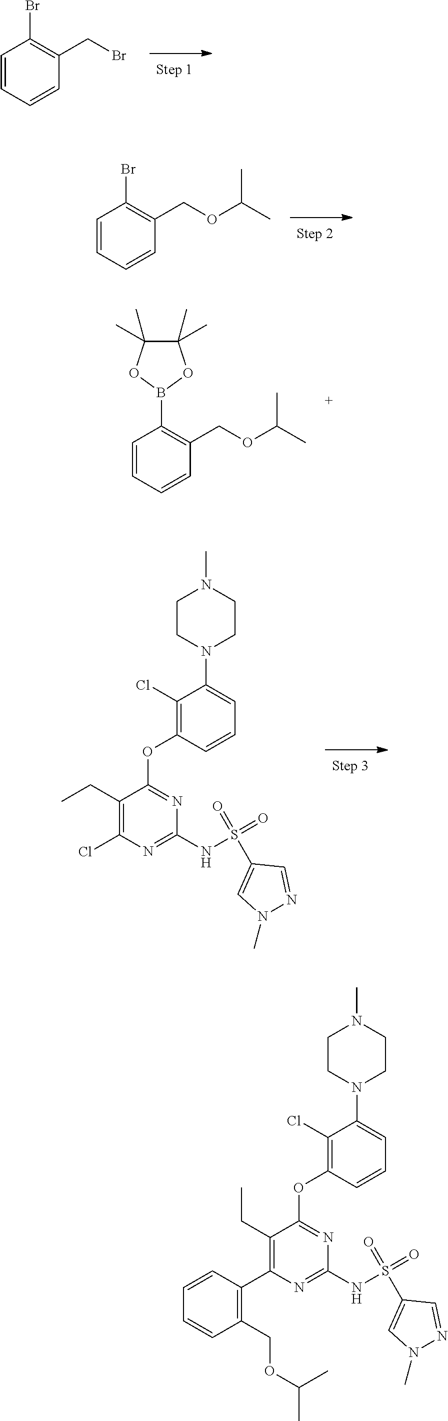

### Step 1: 1-Bromo-2-(isopropoxymethyl)benzene

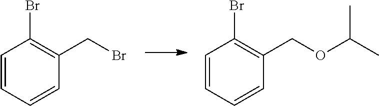

To a solution of isopropanol (3.60 g, 60 mmol) in anhydrous tetrahydrofuran (140 mL) at 0° C. was added a 60% suspension of sodium hydride in mineral oil (2.4 g, 60 mmol) and the mixture was stirred at this temperature for 30 minutes. 1-Bromo-2-(bromomethyl)benzene (10 g, 40 mmol) was added dropwise. The reaction mixture was slowly warmed to ambient temperature and stirred for 48 hours. Diethyl ether (800 mL) and water (100 mL) were added. The organic layer was separated, washed with brine (2×100 mL), dried over magnesium sulfate and concentrated. The residue was purified by silica gel column chromatography using 0-20% hexanes-dichloromethane to afford 1-bromo-2-(isopropoxymethyl)benzene (8.32 g, 90%) as a colorless oil. ¹H NMR (250 MHz, CDCl₃) δ (ppm): 7.53 (m, 2H), 7.31 (m, 1H), 7.16 (m, 1H), 4.57 (s, 2H), 3.75 (m, 1H), 1.26 (d, J=6.0 Hz, 6H). ESI-MS m/z calc. 228.01498, Retention time: 5.74 minutes;

### Step 2: 2-[2-(Isopropoxymethyl)phenyl]-4,4,5,5-tetramethyl-1,3,2-dioxaborolane

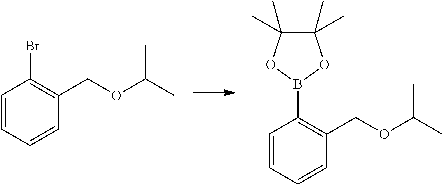 [SMILES: CC(C)OCc1ccccc1Br ; CC(C)OCc1ccccc1B1OC(C)(C)C(C)(C)O1]

Argon gas was bubbled through a mixture of 1-bromo-2-(isopropoxymethyl)benzene (8.33 g, 36.35 mmol), 4,4,5,5-tetramethyl-2-(4,4,5,5-tetramethyl-1,3,2-dioxaborolan-2-yl)-1,3,2-dioxaborolane (10.15 g, 39.99 mmol), potassium acetate (10.7 g, 0.109 mol) and 1,1′-bis(diphenylphosphino)ferrocene-palladium(II)dichloride dichloromethane complex (1.48 g, 1.82 mmol) in anhydrous dioxane (181 mL) for 10 minutes. The reaction vessel was sealed, and the reaction mixture was stirred at 80° C. for 19 hours. Diethyl ether (900 mL) and water (100 mL) were added. The organic layer was separated, washed with brine (2×50 mL), dried over magnesium sulfate and concentrated. The residue was purified by silica gel column chromatography using 0-5% hexanes-diethyl ether to afford crude 5.1 g of crude material, contaminated with the ligand. The crude was purified by reverse phase column chromatography using 0-100% water-acetonitrile (0.1% trifluoroacetic acid). The pure fractions were combined, neutralized with saturated aqueous sodium bicarbonate solution and concentrated under reduced pressure to remove most of the acetonitrile. The aqueous residue was extracted with diethyl ether (3×50 mL). The organic layer was dried over magnesium sulfate and concentrated to afford 2-(2-(isopropoxymethyl)phenyl)-4,4,5,5-tetramethyl-1,3,2-dioxaborolane (3.16 g, 33%) as a pale yellow liquid. ¹H NMR (250 MHz, DMSO) δ (ppm): 7.63 (m, 1H), 7.44 (m, 2H), 7.27 (m, 1H), 4.63 (s, 2H), 3.62 (m, 1H), 1.30 (s, 12H), 1.13 (d, J=6.0 Hz, 6H). ESI-MS m/z calc. 276.18967, Retention time: 3.23 minutes.

### Step 3: N-[4-[2-chloro-3-(4-methylpiperazin-1-yl)phenoxy]-5-ethyl-6-[2-(isopropoxymethyl)phenyl]pyrimidin-2-yl]-1-methyl-pyrazole-4-sulfonamide

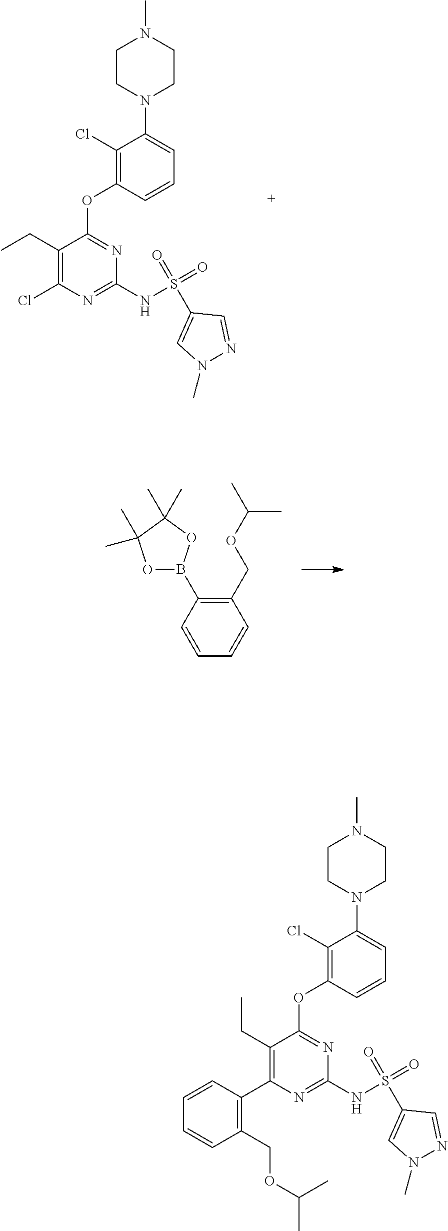 [SMILES: CCc1c(Cl)nc(NS(=O)(=O)c2cnn(C)c2)nc1Oc1cccc(N2CCN(C)CC2)c1Cl ; CC(C)OCc1ccccc1B1OC(C)(C)C(C)(C)O1 ; CCc1c(Oc2cccc(N3CCN(C)CC3)c2Cl)nc(NS(=O)(=O)c2cnn(C)c2)nc1-c1ccccc1COC(C)C]

N-[4-chloro-6-[2-chloro-3-(4-methylpiperazin-1-yl)phenoxy]-5-ethyl-pyrimidin-2-yl]-1-methyl-pyrazole-4-sulfonamide (21.9 mg, 0.03910 mmol), 2-[2-(isopropoxymethyl)phenyl]-4,4,5,5-tetramethyl-1,3,2-dioxaborolane (18 mg, 0.06518 mmol), tetrakis(triphenylphosphine)palladium (0) (6.3 mg, 0.005452 mmol), and 2 M aqueous potassium carbonate (100 μL of 2 M, 0.2000 mmol) were combined in dioxane (900 μL) and heated in a sealed vial for 1 hours at 120° C. The reaction was filtered and purified by LC/MS utilizing a gradient of 1-99% acetonitrile in 5 mM aqueous HCl to yield N-[4-[2-chloro-3-(4-methylpiperazin-1-yl)phenoxy]-5-ethyl-6-[2-(isopropoxymethyl)phenyl]pyrimidin-2-yl]-1 -methyl-pyrazole-4-sulfonamide (hydrochloride salt) (8 mg, 29%). ESI-MS m/z calc. 639.23944, found 640.5 (M+1)⁺; Retention time: 1.4 minutes. LC method A.

### Example 368: Preparation of Compound 1350

### Step 1:N-[5-ethyl-4-[2-fluoro-3-(4-methylpiperazin-1-yl)phenoxy]-6-(2-isopropylphenyl)pyrimidin-2-yl]-1-methyl-pyrazole-4-sulfonamide

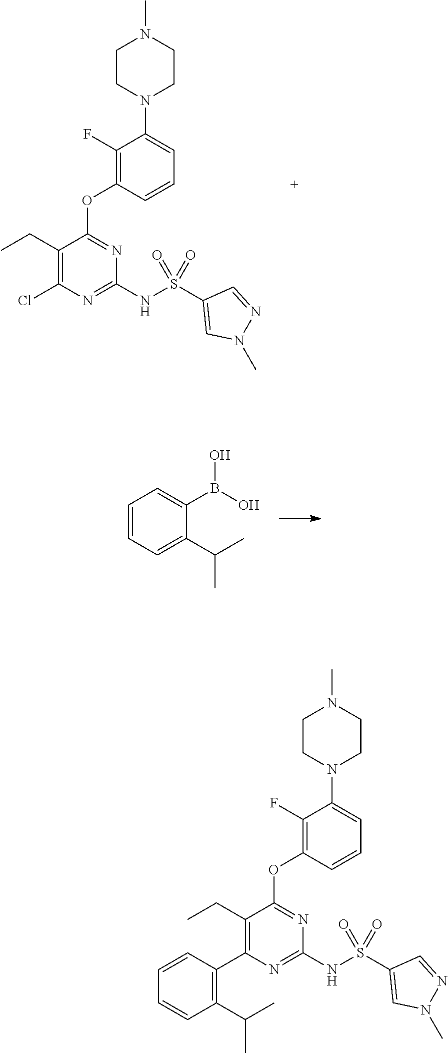 [SMILES: CCc1c(Cl)nc(NS(=O)(=O)c2cnn(C)c2)nc1Oc1cccc(N2CCN(C)CC2)c1F ; CC(C)c1ccccc1B(O)O ; CCc1c(Oc2cccc(N3CCN(C)CC3)c2F)nc(NS(=O)(=O)c2cnn(C)c2)nc1-c1ccccc1C(C)C]

The compound was prepared in a manner analogous to that described above using commercially available (2-isopropylphenyl)boronic acid to give N-[5-ethyl-4-[2-fluoro-3-(4-methylpiperazin-1-yl)phenoxy]-6-(2-isopropylphenyl)pyrimidin-2-yl]-1-methyl-pyrazole-4-sulfonamide (hydrochloride salt) (0.6 mg, 3%). ESI-MS m/z calc. 593.2584, found 594.33 (M+1)⁺; Retention time: 1.49 minutes. LC method A.

### Example 369: Preparation of Compound 1351

### Step 1: N-[4-[2-chloro-3-(4-methylpiperazin-1-yl)phenoxy]-6-(2-ethoxyphenyl)-5-ethyl-pyrimidin-2-yl]-1-methyl-pyrazole-4-sulfonamide

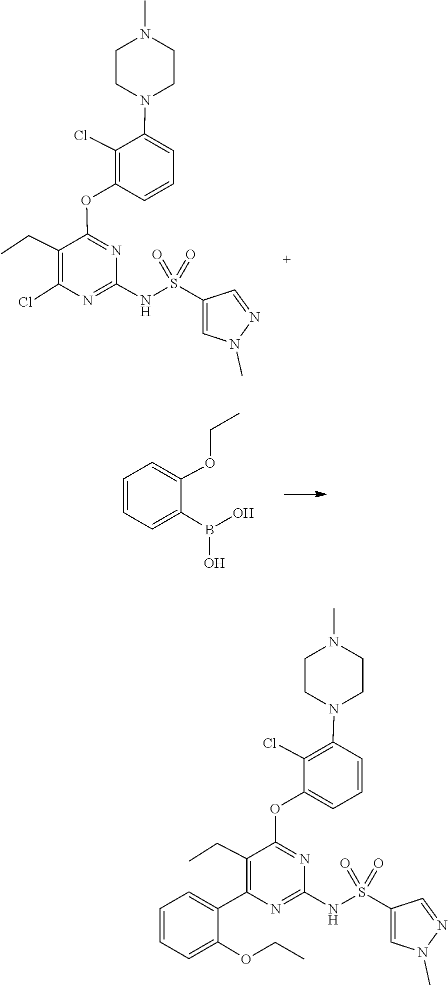

The compound was prepared in a manner analogous to that described above using commercially available (2-ethoxyphenyl)boronic acid to give N-[4-[2-chloro-3-(4-methylpiperazin-1-yl)phenoxy]-6-(2-ethoxyphenyl)-5-ethyl-pyrimidin-2-yl]-1-methyl-pyrazole-4-sulfonamide (hydrochloride salt) (4.5 mg, 13%). ESI-MS m/z calc. 611.2081, found 612.3 (M+1)⁺; Retention time: 1.26 minutes; LC method A.

### Example 370: Preparation of Compound 1352

### Step 1: N-[4-[2-chloro-3-(4-methylpiperazin-1-yl)phenoxy]-5-ethyl-6-(2-isopropoxyphenyl)pyrimidin-2-yl]-1-methyl-pyrazole-4-sulfonamide

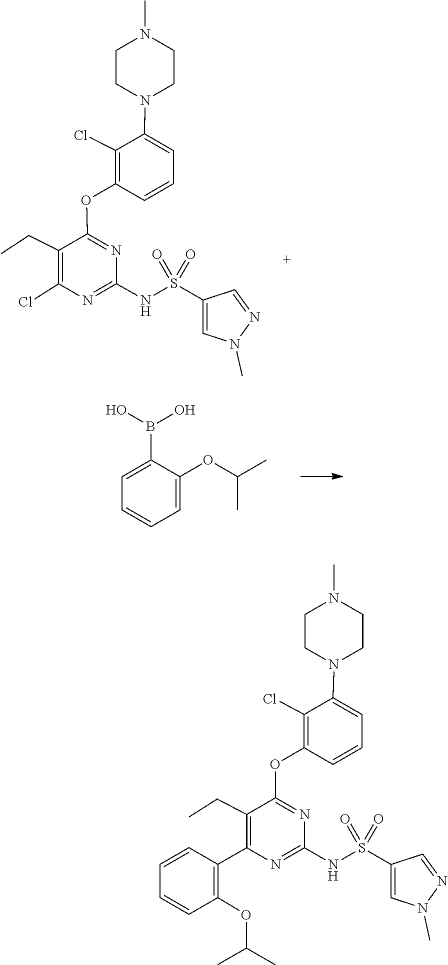 [SMILES: CCc1c(Cl)nc(NS(=O)(=O)c2cnn(C)c2)nc1Oc1cccc(N2CCN(C)CC2)c1Cl ; CC(C)Oc1ccccc1B(O)O ; CCc1c(Oc2cccc(N3CCN(C)CC3)c2Cl)nc(NS(=O)(=O)c2cnn(C)c2)nc1-c1ccccc1OC(C)C]

The compound was prepared in a manner analogous to that described above using commercially available (2-isopropoxyphenyl)boronic acid to give N-[4-[2-chloro-3-(4-methylpiperazin-1-yl)phenoxy]-5-ethyl-6-(2-isopropoxyphenyl)pyrimidin-2-yl]-1-methyl-pyrazole-4-sulfonamide (hydrochloride salt) (9.1 mg, 29%) ESI-MS m/z calc. 625.2238, found 626.3 (M+1)⁺; Retention time: 1.47 minutes. LC method A.

### Example 371: Preparation of Compound 1353

### Step 1: N-[4-[2-chloro-3-(4-methylpiperazin-1-yl)phenoxy]-5-ethyl-6-phenyl-pyrimidin-2-yl]-1-methyl-pyrazole-4-sulfonamide

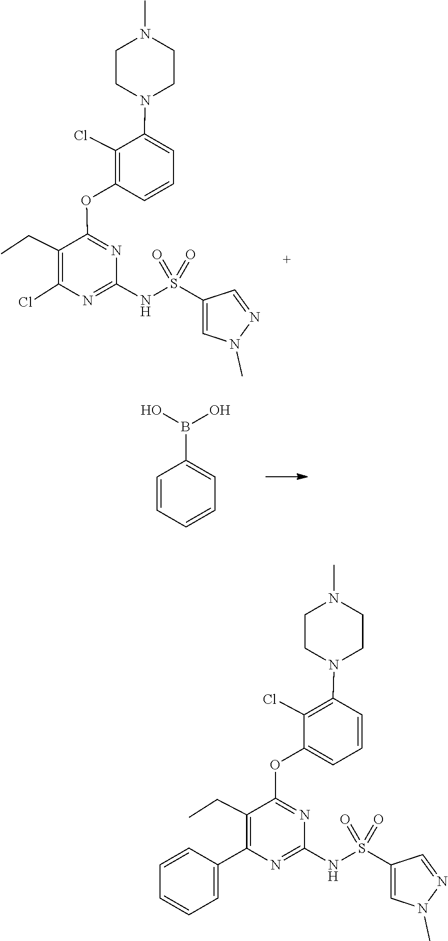

The compound was prepared in a manner analogous to that described above using commercially available phenylboronic acid to give N-[4-[2-chloro-3-(4-methylpiperazin-1-yl)phenoxy]-5-ethyl-6-phenyl-pyrimidin-2-yl]-1-methyl-pyrazole-4-sulfonamide (hydrochloride salt) (6.2 mg, 18%). ESI-MS m/z calc. 567.18195, found 568.3 (M+1)⁺; Retention time: 1.65 minutes; LC method A.

### Example 372: Preparation of Compound 1321

### Step 1: N-[5-ethyl-4,6-bis(o-tolyl)pyrimidin-2-yl]-1-methyl-pyrazole-4-sulfonamide

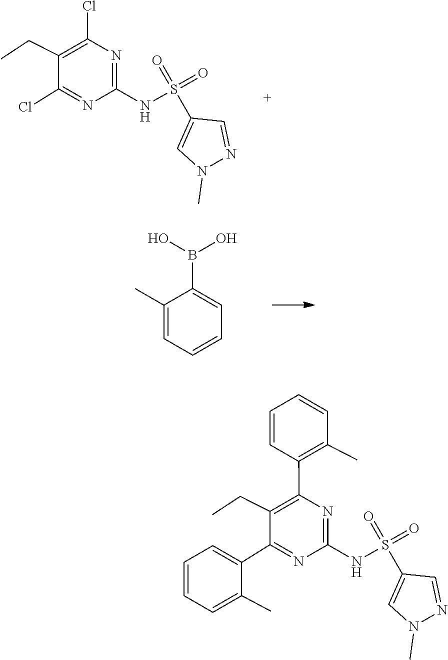

A mixture of N-(4,6-dichloro-5-ethyl-pyrimidin-2-yl)-1-methyl-pyrazole-4-sulfonamide (200 mg, 0.5949 mmol), o-tolylboronic acid (89.7 mg, 0.6598 mmol), Pd(dppf)Cl₂ (45.1 mg, 0.06164 mmol), and a potassium carbonate solution (629 μL of 2 M, 1.258 mmol) in 1,2-dimethoxyethane (1.3 mL) was degassed by flow of nitrogen and the reaction was stirred at 130° C. for 10 minutes in a pressure vessel. The reaction was filtered. EtOAc and water were added to the reaction and acidified with 1 N HCl. The two layers were separated, and the aqueous layer was extracted with EtOAc (×2). The combined organic layer was dried over Na₂SO₄, filtered and concentrated. The crude material was purified on 40 g of silica gel utilizing a gradient of 0-100% ethyl acetate in hexane to yield N-[5-ethyl-4,6-bis(o-tolyl)pyrimidin-2-yl]-1-methyl-pyrazole-4-sulfonamide (43 mg, 16%).¹H NMR (400 MHz, DMSO-d₆) δ 11.57 (s, 1H), 8.08 (s, 1H), 7.62 (s, 1H), 7.41 (dd, J=7.7, 1.5 Hz, 1H), 7.39-7.35 (m, 3H), 7.33 (td, J=6.8, 6.3, 2.4 Hz, 2H), 7.27 (dd, J=7.4, 1.3 Hz, 2H), 3.77 (s, 3H), 2.21-1.93 (m, 8H), 0.55 (t, J=7.4 Hz, 3H). ESI-MS m/z calc. 447.17288, found 448.2 (M+1)⁺; Retention time: 1.89 minutes (LC method A).

### Example 373: Preparation of Compound 1355

### Step 1: N-[5-ethyl-4-(o-tolyl)-6-(4-piperazin-1-ylphenoxy)pyrimidin-2-yl]-1-methyl-pyrazole-4-sulfonamide

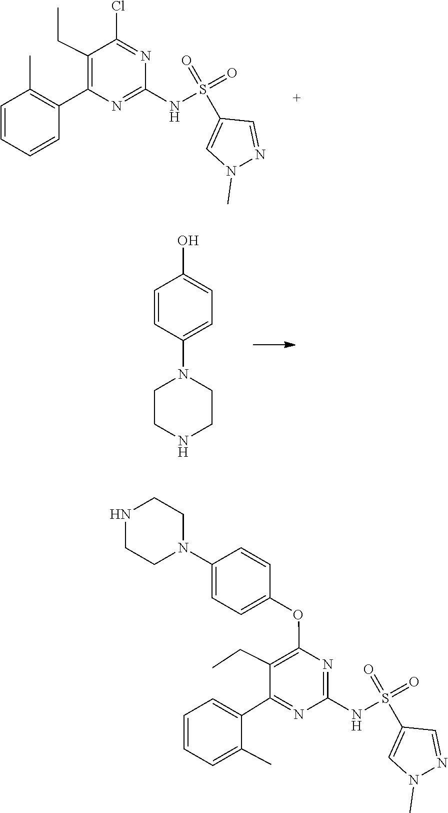 [SMILES: CCc1c(Cl)nc(NS(=O)(=O)c2cnn(C)c2)nc1-c1ccccc1C ; Oc1ccc(N2CCNCC2)cc1 ; CCc1c(Oc2ccc(N3CCNCC3)cc2)nc(NS(=O)(=O)c2cnn(C)c2)nc1-c1ccccc1C]

A heterogenous solution of N-[4-chloro-5-ethyl-6-(o-tolyl)pyrimidin-2-yl]-1-methyl-pyrazole-4-sulfonamide (20 mg, 0.05104 mmol), 4-piperazin-1-ylphenol (approximately 27.29 mg, 0.1531 mmol), and cesium carbonate (approximately 83.15 mg, 0.2552 mmol) in NMP (400μL) was sealed in a vial and the reaction was heated to 110° C. for 16 hours. The reaction was diluted with DMSO, filtered, and purified by reverse phase chromatography to give N-[5-ethyl-4-(o-tolyl)-6-(4-piperazin-1-ylphenoxy)pyrimidin-2-yl]-1-methyl-pyrazole-4-sulfonamide (8.9 mg, 31%). ESI-MS m/z calc. 533.2209, found 534.14 (M+1)⁺; Retention time: 0.95 minutes; LC method A.

### Example 374: Preparation of Compound 1356

### Step 1: N-[4-(2,6-Dimethylphenyl)-5-ethyl-6-(4-piperazin-1-ylphenoxy)pyrimidin-2-yl]-1-methyl-pyrazole-4-sulfonamide

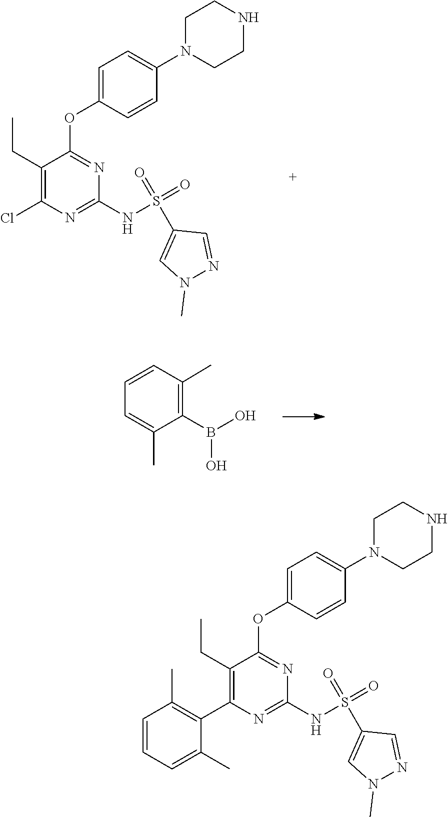 [SMILES: CCc1c(Cl)nc(NS(=O)(=O)c2cnn(C)c2)nc1Oc1ccc(N2CCNCC2)cc1 ; Cc1cccc(C)c1B(O)O ; CCc1c(Oc2ccc(N3CCNCC3)cc2)nc(NS(=O)(=O)c2cnn(C)c2)nc1-c1c(C)cccc1C]

The compound was prepared in a manner analogous to that described above using commercially available (2,6-dimethylphenyl)boronic acid to give N-[4-(2,6-dimethylphenyl)-5-ethyl-6-(4-piperazin-1-ylphenoxy)pyrimidin-2-yl]-1-methyl-pyrazole-4-sulfonamide (2.8 mg, 5%). ESI-MS m/z calc. 547.2366, found 548.54 (M+1)⁺; Retention time: 1.33 minutes; LC method A.

### Example 375: Characterization of Compounds 1357-1402

The compounds in the following tables were prepared in a manner analogous to that described above using commercially available reagents and intermediates described herein.

| | | LCMS | | | |
|---|---|---|---|---|---|
| | | Room | | | |
| Compound | | temperature | Calc. | | LCMS |
| number | Structure | (min) | mass | M + 1 | Method |
| 1357 | 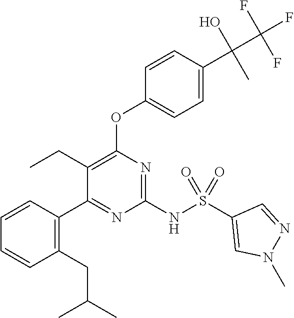 [SMILES: CCc1c(Oc2ccc(C(C)(O)C(F)(F)F)cc2)nc(NS(=O)(=O)c2cnn(C)c2)nc1-c1ccccc1CC(C)C] | 2.08 | 603.213 | 604.23 | A |
| 1358 | 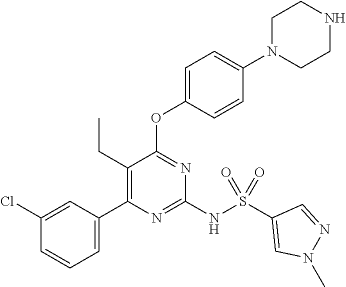 [SMILES: CCc1c(Oc2ccc(N3CCNCC3)cc2)nc(NS(=O)(=O)c2cnn(C)c2)nc1-c1cccc(Cl)c1] | 1.02 | 553.166 | 554.25 | R |
| 1359 | 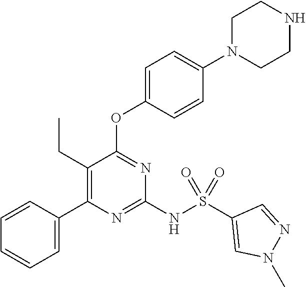 [SMILES: CCc1c(Oc2ccc(N3CCNCC3)cc2)nc(NS(=O)(=O)c2cnn(C)c2)nc1-c1ccccc1] | 0.89 | 519.205 | 520.27 | R |
| 1360 | 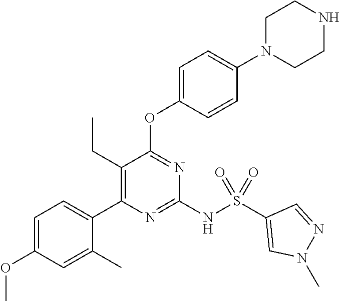 [SMILES: CCc1c(Oc2ccc(N3CCNCC3)cc2)nc(NS(=O)(=O)c2cnn(C)c2)nc1-c1ccc(OC)cc1C] | 1.28 | 563.231 | 564.51 | A |
| 1361 | 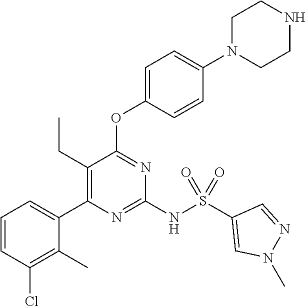 [SMILES: CCc1c(Oc2ccc(N3CCNCC3)cc2)nc(NS(=O)(=O)c2cnn(C)c2)nc1-c1cccc(Cl)c1C] | 1.46 | 567.182 | 568.5 | A |
| 1362 | 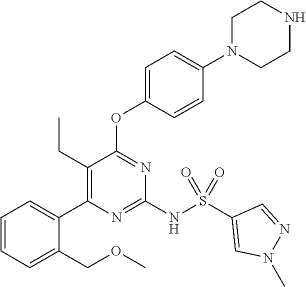 [SMILES: CCc1c(Oc2ccc(N3CCNCC3)cc2)nc(NS(=O)(=O)c2cnn(C)c2)nc1-c1ccccc1COC] | 0.88 | 563.231 | 564.3 | R |
| 1363 | 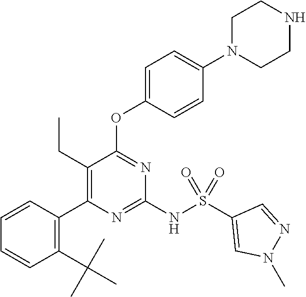 [SMILES: CCc1c(Oc2ccc(N3CCNCC3)cc2)nc(NS(=O)(=O)c2cnn(C)c2)nc1-c1ccccc1C(C)(C)C] | 1.04 | 575.268 | 575.18 | R |
| 1364 | 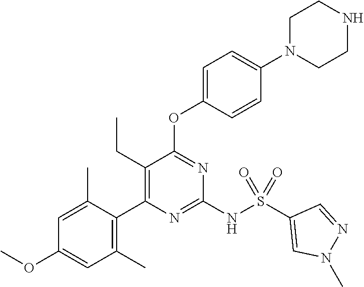 [SMILES: CCc1c(Oc2ccc(N3CCNCC3)cc2)nc(NS(=O)(=O)c2cnn(C)c2)nc1-c1c(C)cc(OC)cc1C] | 0.94 | 577.247 | 578.33 | R |
| 1365 | 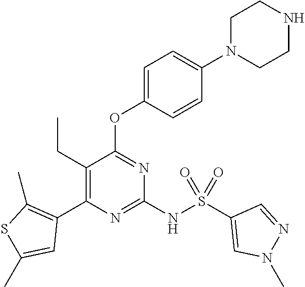 [SMILES: CCc1c(Oc2ccc(N3CCNCC3)cc2)nc(NS(=O)(=O)c2cnn(C)c2)nc1-c1cc(C)sc1C] | 1.38 | 553.193 | 554.49 | A |
| 1366 | 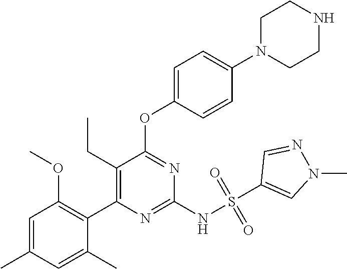 | 0.99 | 577.247 | 578.33 | R |
| 1367 | 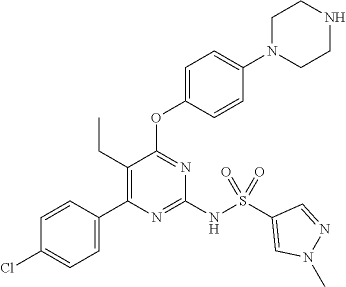 [SMILES: CCc1c(Oc2ccc(N3CCNCC3)cc2)nc(NS(=O)(=O)c2cnn(C)c2)nc1-c1ccc(Cl)cc1] | 1.4 | 553.166 | 554.46 | A |
| 1368 | 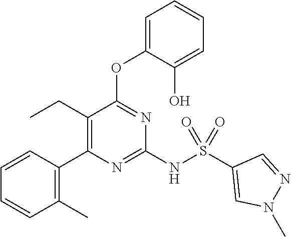 [SMILES: CCc1c(Oc2ccccc2O)nc(NS(=O)(=O)c2cnn(C)c2)nc1-c1ccccc1C] | 1.5 | 465.147 | 466.47 | A |
| 1369 | 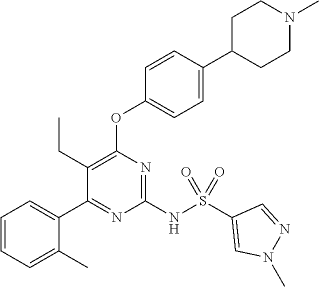 [SMILES: CCc1c(Oc2ccc(C3CCN(C)CC3)cc2)nc(NS(=O)(=O)c2cnn(C)c2)nc1-c1ccccc1C] | 1.38 | 546.241 | 547.5 | A |
| 1370 | 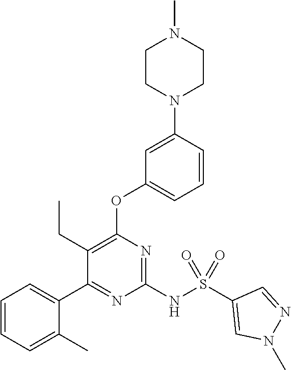 [SMILES: CCc1c(Oc2cccc(N3CCN(C)CC3)c2)nc(NS(=O)(=O)c2cnn(C)c2)nc1-c1ccccc1C] | 1.38 | 547.237 | 548.35 | A |
| 1371 | 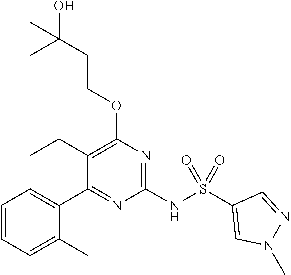 [SMILES: CCc1c(OCCC(C)(C)O)nc(NS(=O)(=O)c2cnn(C)c2)nc1-c1ccccc1C] | 1.36 | 459.194 | 460.13 | A |
| 1372 | 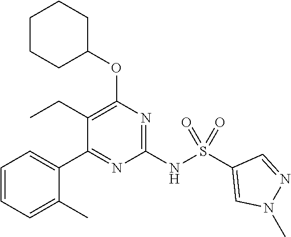 [SMILES: CCc1c(OC2CCCCC2)nc(NS(=O)(=O)c2cnn(C)c2)nc1-c1ccccc1C] | 1.74 | 455.199 | 456.17 | A |
| 1373 | 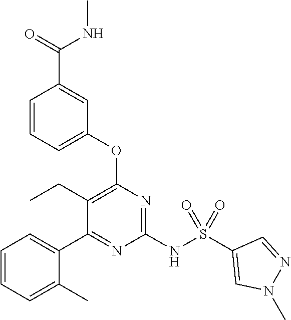 [SMILES: CCc1c(Oc2cccc(C(=O)NC)c2)nc(NS(=O)(=O)c2cnn(C)c2)nc1-c1ccccc1C] | 1.43 | 506.174 | 507.11 | A |
| 1374 | 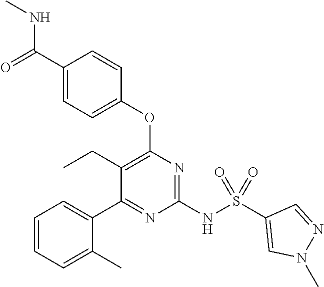 [SMILES: CCc1c(Oc2ccc(C(=O)NC)cc2)nc(NS(=O)(=O)c2cnn(C)c2)nc1-c1ccccc1C] | 1.42 | 506.174 | 507.11 | A |
| 1375 | 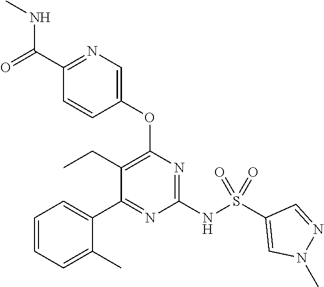 [SMILES: CCc1c(Oc2ccc(C(=O)NC)nc2)nc(NS(=O)(=O)c2cnn(C)c2)nc1-c1ccccc1C] | 1.44 | 507.169 | 508.1 | A |
| 1376 | 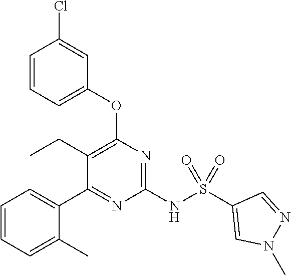 [SMILES: CCc1c(Oc2cccc(Cl)c2)nc(NS(=O)(=O)c2cnn(C)c2)nc1-c1ccccc1C] | 1.8 | 483.113 | 484.05 | A |
| 1377 | 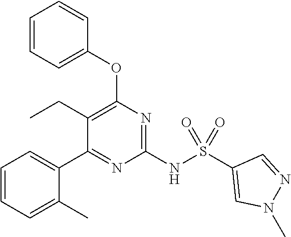 [SMILES: CCc1c(Oc2ccccc2)nc(NS(=O)(=O)c2cnn(C)c2)nc1-c1ccccc1C] | 1.63 | 449.152 | 450.09 | A |
| 1378 | 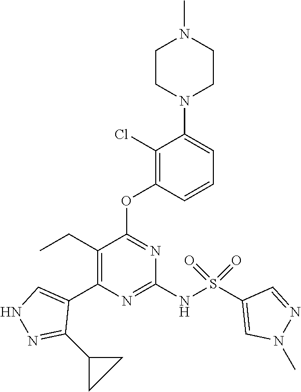 [SMILES: CCc1c(Oc2cccc(N3CCN(C)CC3)c2Cl)nc(NS(=O)(=O)c2cnn(C)c2)nc1-c1c[nH]nc1C1CC1] | 1.01 | 597.204 | 598.22 | A |
| 1379 | 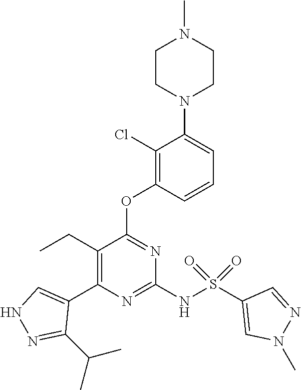 [SMILES: CCc1c(Oc2cccc(N3CCN(C)CC3)c2Cl)nc(NS(=O)(=O)c2cnn(C)c2)nc1-c1c[nH]nc1C(C)C] | 1.04 | 599.219 | 600.2 | A |
| 1380 | 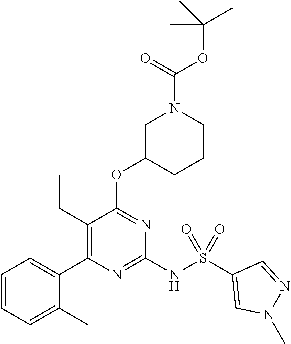 [SMILES: CCc1c(OC2CCCN(C(=O)OC(C)(C)C)C2)nc(NS(=O)(=O)c2cnn(C)c2)nc1-c1ccccc1C] | 1.87 | 556.247 | 557.4 | A |
| 1381 | 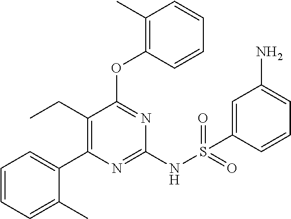 [SMILES: CCc1c(Oc2ccccc2C)nc(NS(=O)(=O)c2cccc(N)c2)nc1-c1ccccc1C] | 1.89 | 474.173 | 475.2 | A |
| 1382 | 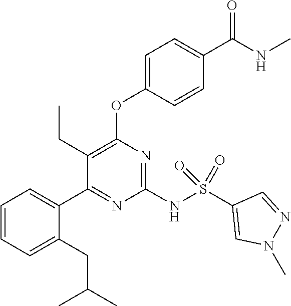 [SMILES: CCc1c(Oc2ccc(C(=O)NC)cc2)nc(NS(=O)(=O)c2cnn(C)c2)nc1-c1ccccc1CC(C)C] | 1.7 | 548.221 | 549.4 | A |
| 1383 | 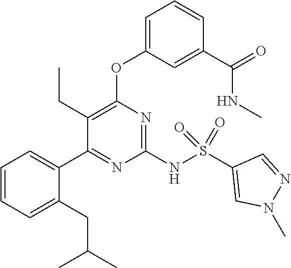 [SMILES: CCc1c(Oc2cccc(C(=O)NC)c2)nc(NS(=O)(=O)c2cnn(C)c2)nc1-c1ccccc1CC(C)C] | 1.8 | 548.221 | 549.3 | A |
| 1384 | 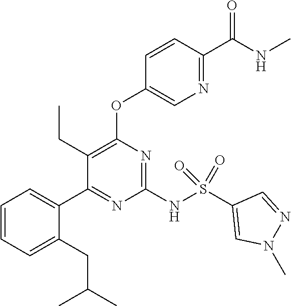 [SMILES: CCc1c(Oc2ccc(C(=O)NC)nc2)nc(NS(=O)(=O)c2cnn(C)c2)nc1-c1ccccc1CC(C)C] | 1.73 | 549.216 | 550.3 | Q |
| 1385 | 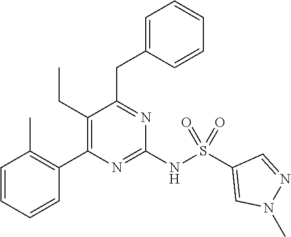 [SMILES: CCc1c(Cc2ccccc2)nc(NS(=O)(=O)c2cnn(C)c2)nc1-c1ccccc1C] | 1.8 | 447.173 | 448.3 | A |
| 1386 | 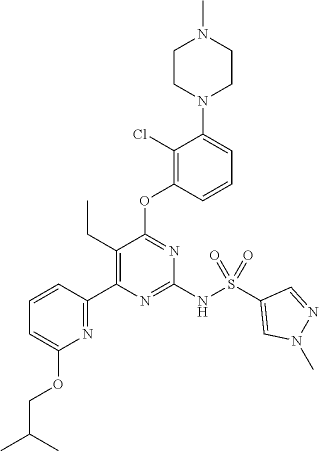 [SMILES: CCc1c(Oc2cccc(N3CCN(C)CC3)c2Cl)nc(NS(=O)(=O)c2cnn(C)c2)nc1-c1cccc(OCC(C)C)n1] | 1.68 | 640.235 | 641.5 | A |
| 1387 | 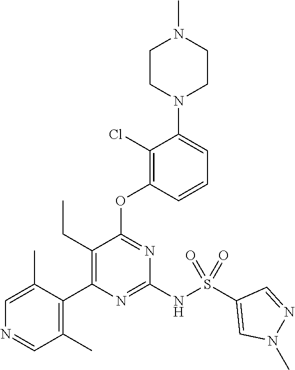 [SMILES: CCc1c(Oc2cccc(N3CCN(C)CC3)c2Cl)nc(NS(=O)(=O)c2cnn(C)c2)nc1-c1c(C)cncc1C] | 0.86 | 596.208 | 597.4 | A |
| 1388 | 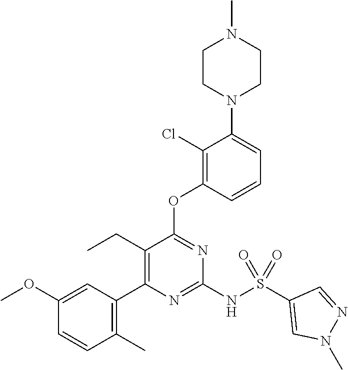 | 1.31 | 611.208 | 612.1 | A |
| 1389 | 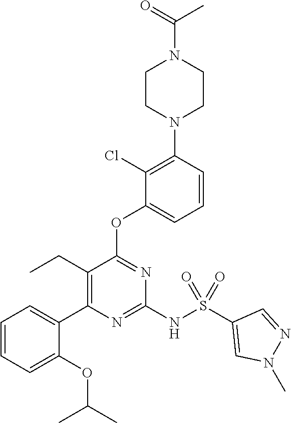 [SMILES: CCc1c(Oc2cccc(N3CCN(C(C)=O)CC3)c2Cl)nc(NS(=O)(=O)c2cnn(C)c2)nc1-c1ccccc1OC(C)C] | 1.75 | 653.219 | 654.3 | A |
| 1390 | 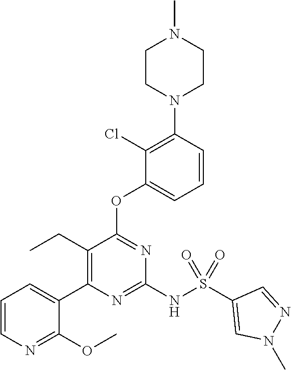 [SMILES: CCc1c(Oc2cccc(N3CCN(C)CC3)c2Cl)nc(NS(=O)(=O)c2cnn(C)c2)nc1-c1cccnc1OC] | 0.89 | 598.188 | 599.29 | A |
| 1391 | 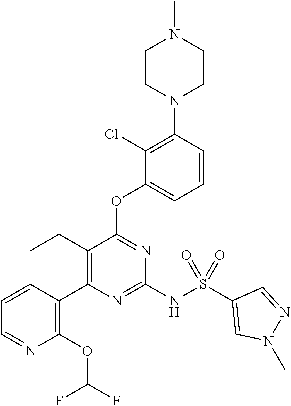 [SMILES: CCc1c(Oc2cccc(N3CCN(C)CC3)c2Cl)nc(NS(=O)(=O)c2cnn(C)c2)nc1-c1cccnc1OC(F)F] | 0.9 | 634.169 | 635.25 | A |
| 1392 | 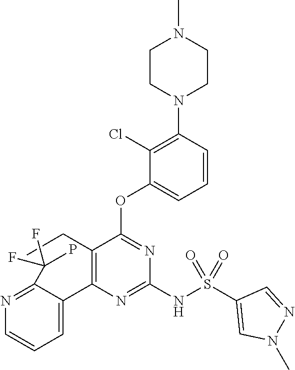 | 0.87 | 636.165 | 637.27 | A |
| 1393 | 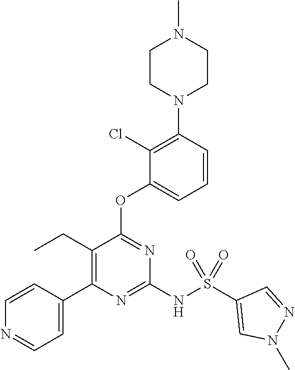 [SMILES: CCc1c(Oc2cccc(N3CCN(C)CC3)c2Cl)nc(NS(=O)(=O)c2cnn(C)c2)nc1-c1ccncc1] | 0.6 | 568.177 | 569.26 | A |
| 1394 | 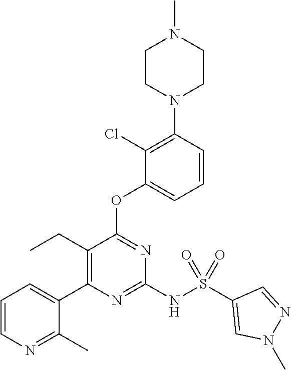 [SMILES: CCc1c(Oc2cccc(N3CCN(C)CC3)c2Cl)nc(NS(=O)(=O)c2cnn(C)c2)nc1-c1cccnc1C] | 0.58 | 582.193 | 583.23 | A |
| 1395 | 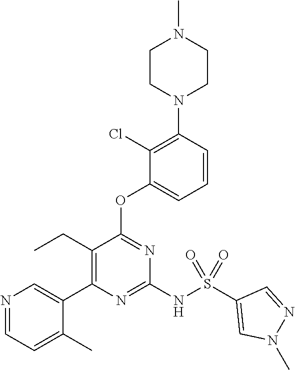 [SMILES: CCc1c(Oc2cccc(N3CCN(C)CC3)c2Cl)nc(NS(=O)(=O)c2cnn(C)c2)nc1-c1cnccc1C] | 0.6 | 582.193 | 583.23 | A |
| 1396 | 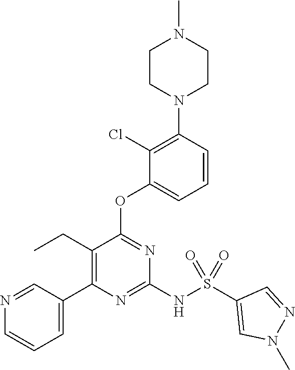 [SMILES: CCc1c(Oc2cccc(N3CCN(C)CC3)c2Cl)nc(NS(=O)(=O)c2cnn(C)c2)nc1-c1cccnc1] | 0.69 | 568.177 | 569.19 | A |
| 1397 | 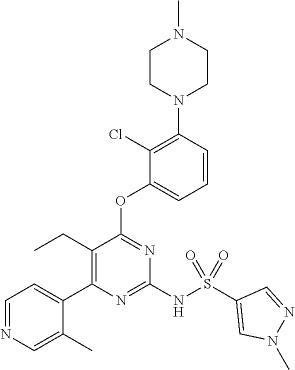 [SMILES: CCc1c(Oc2cccc(N3CCN(C)CC3)c2Cl)nc(NS(=O)(=O)c2cnn(C)c2)nc1-c1ccncc1C] | 0.59 | 582.193 | 583.23 | A |
| 1398 | 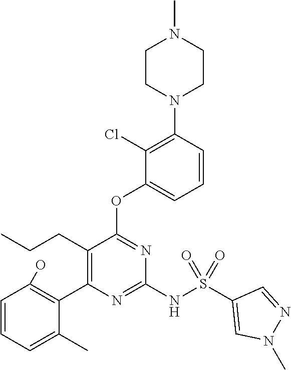 [SMILES: CCCc1c(Oc2cccc(N3CCN(C)CC3)c2Cl)nc(NS(=O)(=O)c2cnn(C)c2)nc1-c1c(C)cccc1O] | 1.23 | 611.208 | 612.3 | A |
| 1399 | 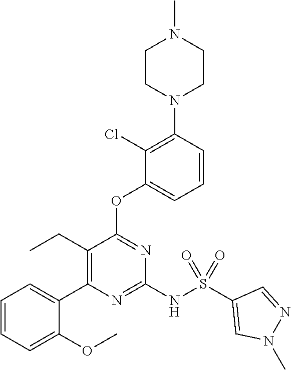 [SMILES: CCc1c(Oc2cccc(N3CCN(C)CC3)c2Cl)nc(NS(=O)(=O)c2cnn(C)c2)nc1-c1ccccc1OC] | 1.16 | 597.193 | 598.3 | A |
| 1400 | 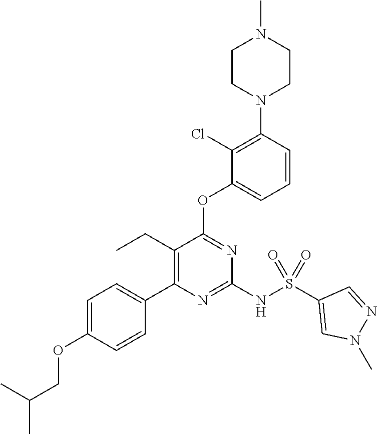 [SMILES: CCc1c(Oc2cccc(N3CCN(C)CC3)c2Cl)nc(NS(=O)(=O)c2cnn(C)c2)nc1-c1ccc(OCC(C)C)cc1] | 1.54 | 639.239 | 640.5 | A |
| 1401 | 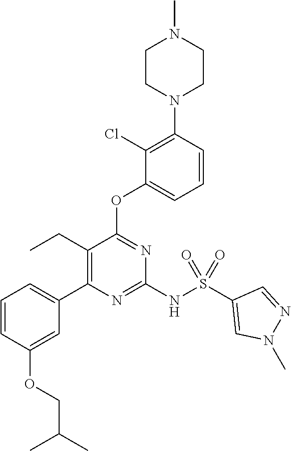 [SMILES: CCc1c(Oc2cccc(N3CCN(C)CC3)c2Cl)nc(NS(=O)(=O)c2cnn(C)c2)nc1-c1cccc(OCC(C)C)c1] | 1.55 | 639.239 | 640.5 | A |
| 1402 | 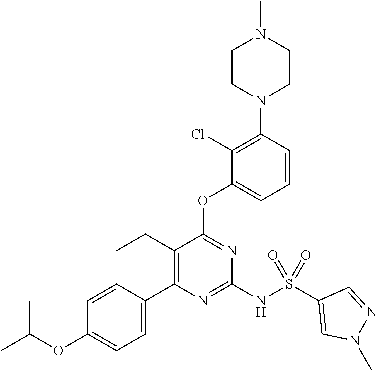 [SMILES: CCc1c(Oc2cccc(N3CCN(C)CC3)c2Cl)nc(NS(=O)(=O)c2cnn(C)c2)nc1-c1ccc(OC(C)C)cc1] | 1.39 | 625.224 | 626.4 | A |

| Compound | |
|---|---|
| number | NMR |
| 1381 | ¹H NMR (400 MHz, DMSO-d₆) δ 11.44 (s, 1H), 7.42 (dd, J = 7.6, 1.8 Hz, 1H), |
| | 7.40 − 7.33 (m, 3H), 7.33 − 7.26 (m, 2H), 7.22 (d, J = 7.5 Hz, 1H), 7.13 (dd, J = |
| | 7.9, 1.4 Hz, 1H), 6.96 (s, 1H), 6.91 (t, J = 7.8 Hz, 1H), 6.80 (d, J = 8.1 Hz, 1H), |
| | 6.28 (d, J = 7.7 Hz, 1H), 2.49 − 2.17 (m, 2H), 2.08 (s, 3H), 2.05 (s, 3H), 0.99 (t, |
| | J = 7.4 Hz, 3H). |

### Example 376: Preparation of Compound 1403

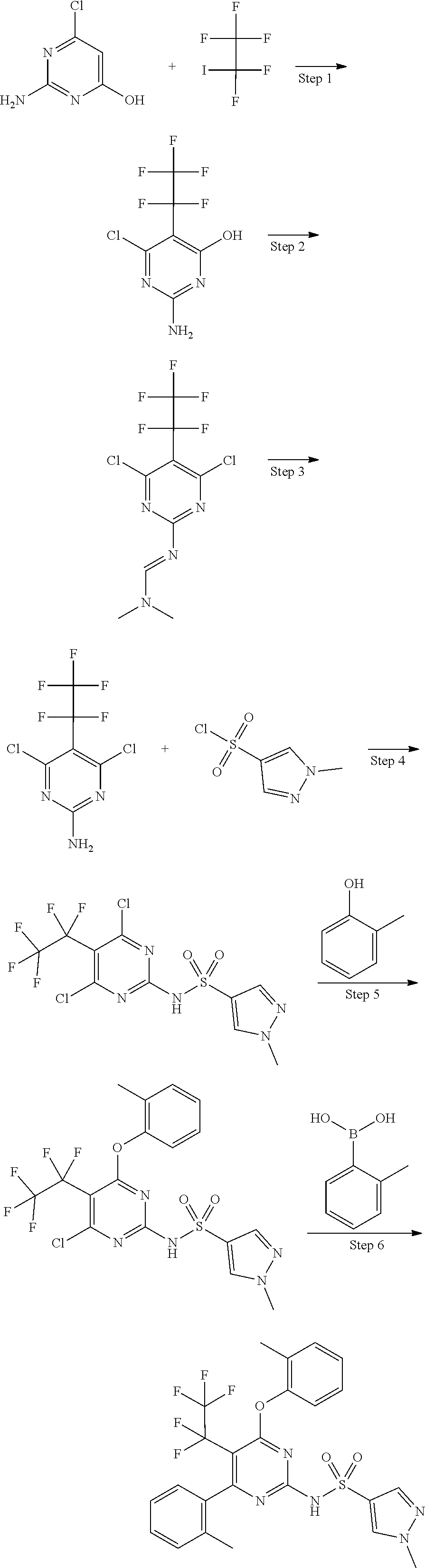

### Step 1: 2-Amino-6-chloro-5-(1,1,2,2,2-pentafluoroethyl)pyrimidin-4-ol

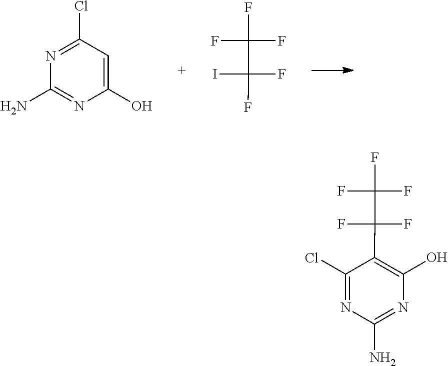 [SMILES: Nc1nc(O)cc(Cl)n1 ; FC(F)(F)C(F)(F)I ; Nc1nc(O)c(C(F)(F)C(F)(F)F)c(Cl)n1]

An empty 500-mL flask was cooled in a dry ice bath and 1,1,1,2,2-pentafluoro-2-iodo-ethane (40.73 g, 165.62 mmol) was condensed inside. The flask was then removed from the cold bath and DMSO (55 mL) was added slowly to give a slurry. This slurry was slowly warmed up until a clear solution was obtained. This solution was then added to a three-neck 500-mL flask containing a solution of 2-amino-6-chloro-pyrimidin-4-ol (12 g, 82.448 mmol) in DMSO (110 mL) at room temperature. Then, a solution of ferrous sulfate was added (25 mL of a roughly 1 M aqueous solution, prepared by dissolving 27.8 g of ferrous sulfate heptahydrate in 100 mL of water, 25 mmol) and the temperature, monitored with a probe inside the reaction, rose a few degrees. To this solution was then added dropwise and carefully aqueous hydrogen peroxide (14 mL of a 35% aqueous solution, 144.06 mmol) cooling the solution with a cold water bath when the internal temperature raised over 35° C. and keeping the internal temperature between 35 and 40° C. during the addition. After another 60 minutes, the crude mixture was transferred to a 3 L flask with water (750 mL)/crushed ice (750 mL) then filtered and dried under high vacuum to afford 2-amino-6-chloro-5-(1,1,2,2,2-pentafluoroethyl)pyrimidin-4-ol (20.54 g, 95%, labelled as VBL00771-098-02) as a white solid. ¹H NMR (300 MHz, DMSO-d₆) δ ppm 6.86 (br. s., 1H), 8.28 (br. s., 1H), 11.70 (br. s., 1H). ¹⁹F NMR (282 MHz, DMSO-d₆) δ ppm −106.9 (s, 2F), -83.5 (s, 3F). ESI-MS m/z calc. 262.9885, found 264.0 (M+1)⁺; Retention time: 1.55 minutes. LC method C.

### Step 2: N′-[4,6-Dichloro-5-(1,1,2,2,2-pentafluoroethyl)pyrimidin-2-yl]-N,N-dimethyl-formamidine

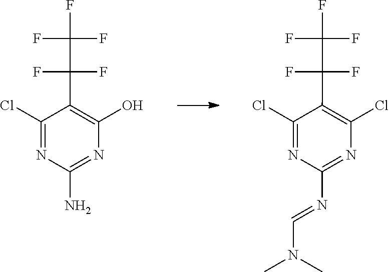 [SMILES: Nc1nc(O)c(C(F)(F)C(F)(F)F)c(Cl)n1 ; CN(C)/C=N/c1nc(Cl)c(C(F)(F)C(F)(F)F)c(Cl)n1]

Oxalyl chloride (162.40 g, 112 mL, 1.2795 mol) was slowly added to a solution of dimethylformamide (93.060 g, 99 mL, 1.2732 mol) in chloroform (1.6 L) and the solution was stirred at room temperature for 30 minutes. 2-Amino-6-chloro-5-(1,1,2,2,2-pentafluoroethyl)pyrimidin-4-ol (56 g, 212.48 mmol) was added then the reaction mixture was heated at 60° C. for 1 hour. Once cooled to room temperature, the reaction mixture was diluted with saturated sodium bicarbonate solution (1.5 L) and stirred vigorously for 15 minutes. 50% Sodium hydroxide solution (50 mL) was added to reach pH ˜8-9. Layers were separated and the aqueous layer was extracted with dichloromethane (2×700 mL). Organic layers were combined, dried over sodium sulfate and concentrated under reduced pressure to afford N′-[4,6-dichloro-5-(1,1,2,2,2-pentafluoroethyl)pyrimidin-2-yl]-N,N-dimethyl-formamidine (278.5 g, 389%) as an orange oil (containing a lot of DMF). ESI-MS m/z calc. 335.9968, found 337.1 (M+1)⁺+; Retention time: 2.0 minutes (LC method C). The crude material was used for next step without any further purification.

### Step 3: 4,6-Dichloro-5-(1,1,2,2,2-pentafluoroethyl)pyrimidin-2-amine

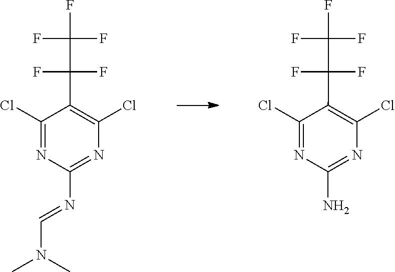 [SMILES: CN(C)/C=N/c1nc(Cl)c(C(F)(F)C(F)(F)F)c(Cl)n1 ; Nc1nc(Cl)c(C(F)(F)C(F)(F)F)c(Cl)n1]

Hydrochloric acid (110 mL of 12 M, 1.3200 mol) was added to a solution of N′-[4,6-dichloro-5-(1,1,2,2,2-pentafluoroethyl)pyrimidin-2-yl]-N,N-dimethyl-formamidine (71.62 g, 212.47 mmol) in isopropanol (1.4 L) and the mixture was stirred at 50° C. for 30 minutes. The solution was concentrated under reduced pressure and ethyl acetate (1.2 L) was added. The organic phase was washed with water (2×500 mL) and brine (500 mL), dried over anhydrous sodium sulfate and concentrated under reduced pressure. The residue was dissolved in isopropanol (500 mL) and 10% hydrochloric acid (100 mL) was added. Isopropanol was removed under reduced pressure and water (500 mL) was added. The mixture was cooled in an ice bath, filtered, rinsed with cold water (2×100 mL) and dried to afford 4,6-dichloro-5-(1,1,2,2,2-pentafluoroethyl)pyrimidin-2-amine (57.04 g, 94%) as an off-white solid. ¹H NMR (300 MHz, DMSO-d₆) δ ppm 8.44 (br s, 2H). ¹⁹F NMR (282 MHz, DMSO-d₆) δ ppm −104.4 (br s, 2F), -82.8 (br s, 3F). ESI-MS m/z calc. 280.9546, found 282.0 (M+1)⁺; Retention time: 2.8 minutes. (LC method M).

### Step 4: N-[4,6-Dichloro-5-(1,1,2,2,2-pentafluoroethyl)pyrimidin-2-yl]-1-methyl-pyrazole-4-sulfonamide

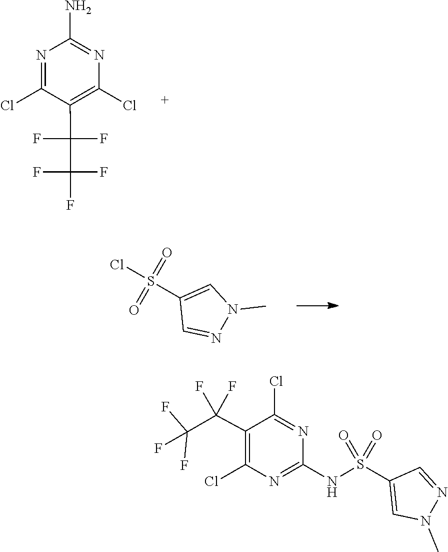

To a solution of 4,6-dichloro-5-(1,1,2,2,2-pentafluoroethyl)pyrimidin-2-amine (3.0 g, 10.64 mmol) in DMF (42.56 mL) at 0° C. was added sodium hydride (approximately 1.702 g of 60% w/w, 42.56 mmol). The reaction was stirred for 15 minutes before adding 1-methylpyrazole-4-sulfonyl chloride (approximately 3.843 g, 21.28 mmol). The reaction was stirred for 15 minutes and quenched with the addition of acetic acid (approximately 6.390 g, 6.051 mL, 106.4 mmol). The crude solution was diluted with water and partitioned with ethyl acetate and brine. The aqueous layer was removed, and the organic layer was further washed with brine (2×). The organic layer was dried over magnesium sulfate, filtered, and concentrated in vacuo. The residual acetic acid was azeotroped twice with benzene. The crude solid was triturated twice with acetone which after drying under vacuum provided N-[4,6-dichloro-5-(1,1,2,2,2-pentafluoroethyl)pyrimidin-2-yl]-1-methyl-pyrazole-4-sulfonamide (3.37 g, 69%) as a light yellow solid. ESI-MS m/z calc. 424.95395, found 426.01 (M+1)⁺; Retention time: 0.63 minutes. LC method D.

### Step 5: N-[4-chloro-6-(2-methylphenoxy)-5-(1,1,2,2,2-pentafluoroethyl)pyrimidin-2-yl]-1-methyl-pyrazole-4-sulfonamide

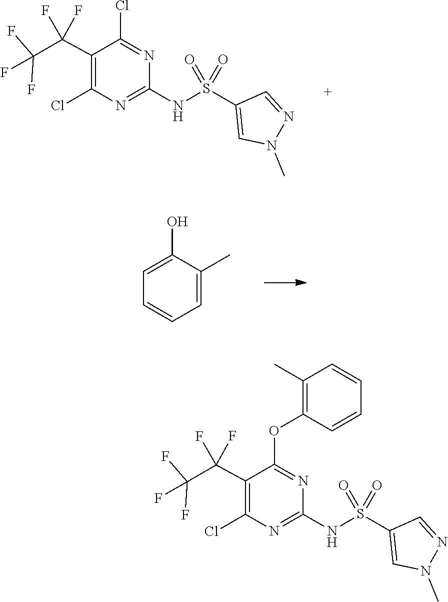

A heterogeneous solution of N-[4,6-dichloro-5-(1,1,2,2,2-pentafluoroethyl)pyrimidin-2-yl]-1-methyl-pyrazole-4-sulfonamide (333 mg, 0.7267 mmol), potassium carbonate (approximately 301.3 mg, 2.180 mmol), and o-cresol (approximately 78.58 mg, 142.1 μL, 0.7267 mmol) in NMP (1.453 mL) was heated to 65° C. for 16 hours. The reaction mixture was diluted with DMSO, acidified with acetic acid (approximately 523.7 mg, 495.9 μL, 8.720 mmol) and filtered. The sample was purified by reverse phase HPLC (Waters Sunfire C₁₈ column (100 ×50 mm, 10 m particle size), gradient: 1-99% acetonitrile in water (5 mM HCl) over 15.0 minutes) to afford N-[4-chloro-6-(2-methylphenoxy)-5-(1,1,2,2,2-pentafluoroethyl)pyrimidin-2-yl]-1-methyl-pyrazole-4-sulfonamide (150 mg, 41%) as a white solid. ESI-MS m/z calc. 497.0348, Retention time: minutes.

### Step 6: 1-methyl-N-[4-(2-methylphenoxy)-6-(o-tolyl)-5-(1,1,2,2,2-pentafluoroethyl)pyrimidin-2-yl]pyrazole-4-sulfonamide

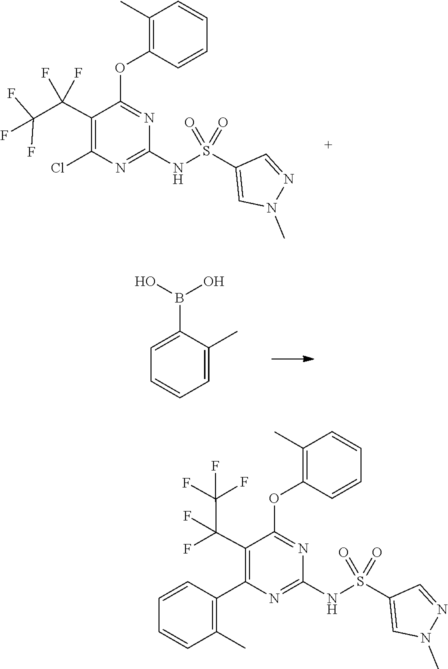

A heterogeneous solution of N-[4-chloro-6-(2-methylphenoxy)-5-(1,1,2,2,2-pentafluoroethyl)pyrimidin-2-yl]-1-methyl-pyrazole-4-sulfonamide (30 mg, 0.06026 mmol), tetrakis(triphenylphosphine)palladium(0) (13.9 mg, 0.01203 mmol), potassium carbonate (25.0 mg, 0.1809 mmol), and o-tolylboronic acid (16.4 mg, 0.1206 mmol) in dioxane (250 μL) and water (50 μL) was microwaved in a sealed vial to 125° C. for 30 minutes. The reaction mixture was acidified with acetic acid (35 μL, 0.6155 mmol), diluted with DMSO (0.5 mL) and filtered through a 0.45 m PTFE syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford 1-methyl-N-[4-(2-methylphenoxy)-6-(o-tolyl)-5-(1,1,2,2,2-pentafluoroethyl)pyrimidin-2-yl]pyrazole-4-sulfonamide (6.9 mg, 21%) as a white solid. ESI-MS m/z calc. 553.1207, found 554.25 (M+1)⁺; Retention time: 2.12 minutes LC method A.

### Example 377: Preparation of Compound 1404

### Step 1: N-[4-chloro-6-[2-chloro-3-(4-methylpiperazin-1-yl)phenoxy]-5-(1,1,2,2,2-pentafluoroethyl)pyrimidin-2-yl]-1-methyl-pyrazole-4-sulfonamide

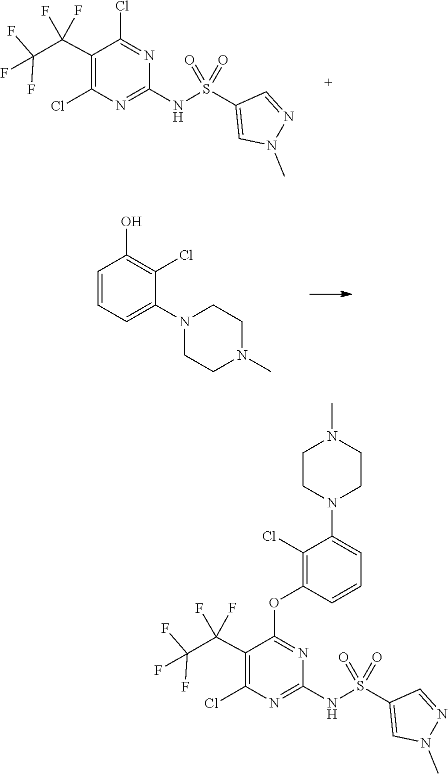 [SMILES: Cn1cc(S(=O)(=O)Nc2nc(Cl)c(C(F)(F)C(F)(F)F)c(Cl)n2)cn1 ; CN1CCN(c2cccc(O)c2Cl)CC1 ; CN1CCN(c2cccc(Oc3nc(NS(=O)(=O)c4cnn(C)c4)nc(Cl)c3C(F)(F)C(F)(F)F)c2Cl)CC1]

A heterogeneous solution of N-[4,6-dichloro-5-(1,1,2,2,2-pentafluoroethyl)pyrimidin-2-yl]-1-methyl-pyrazole-4-sulfonamide (333 mg, 0.7267 mmol), potassium carbonate (300 mg, 2.171 mmol), and 2-chloro-3-(4-methylpiperazin-1-yl)phenol (165 mg, 0.7278 mmol) in NMP (1.5 mL) was heated in a sealed vial to 110° C. for 16 hours. The solution was acidified with acetic acid (500 μL, 8.792 mmol), diluted with water (0.20 mL), and filtered through a 0.45 m PTFE syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-chloro-6-[2-chloro-3-(4-methylpiperazin-1-yl)phenoxy]-5-(1,1,2,2,2-pentafluoroethyl)pyrimidin-2-yl]-1-methyl-pyrazole-4-sulfonamide (hydrochloride salt) (160 mg, 34%) as an orange solid. ESI-MS m/z calc. 615.0646, found 616.11 (M+1)⁺; Retention time: 0.58 minutes (LC method D).

### Step 2: N-[4-[2-chloro-3-(4-methylpiperazin-1-yl)phenoxy]-6-(o-tolyl)-5-(1,1,2,2,2-pentafluoroethyl)pyrimidin-2-yl]-1-methyl-pyrazole-4-sulfonamide

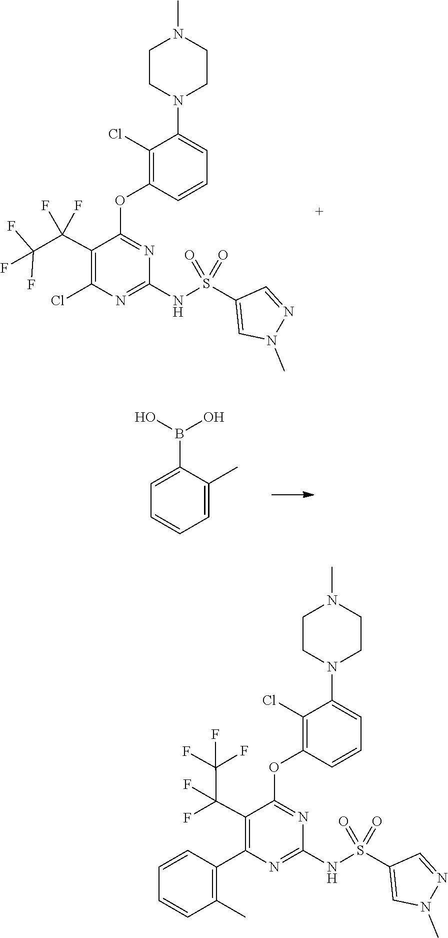

A heterogeneous solution of N-[4-chloro-6-[2-chloro-3-(4-methylpiperazin-1-yl)phenoxy]-5-(1,1,2,2,2-pentafluoroethyl)pyrimidin-2-yl]-1-methyl-pyrazole-4-sulfonamide (hydrochloride salt) (30.0 mg, 0.04595 mmol), o-tolylboronic acid (12.5 mg, 0.09194 mmol), tetrakis(triphenylphosphine)palladium(0) (10.6 mg, 0.009173 mmol), and potassium carbonate (19.0 mg, 0.1375 mmol) in dioxane (200 μL) and water (40 μL) was microwaved in a sealed vial to 125° C. for 30 minutes. The reaction solution was acidified with acetic acid (50 μL, 0.8792 mmol), diluted with DMSO (0.5 mL) and filtered through a 0.45 m PTFE syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-[2-chloro-3-(4-methylpiperazin-1-yl)phenoxy]-6-(o-tolyl)-5-(1,1,2,2,2-pentafluoroethyl)pyrimidin-2-yl]-1-methyl-pyrazole-4-sulfonamide (hydrochloride salt) (14.3 mg, 44%) as a white solid. ESI-MS m/z calc. 671.15045, found 672.25 (M+1)⁺; Retention time: 1.58 minutes, LC method A.

### Example 378: Preparation of Compound 1405

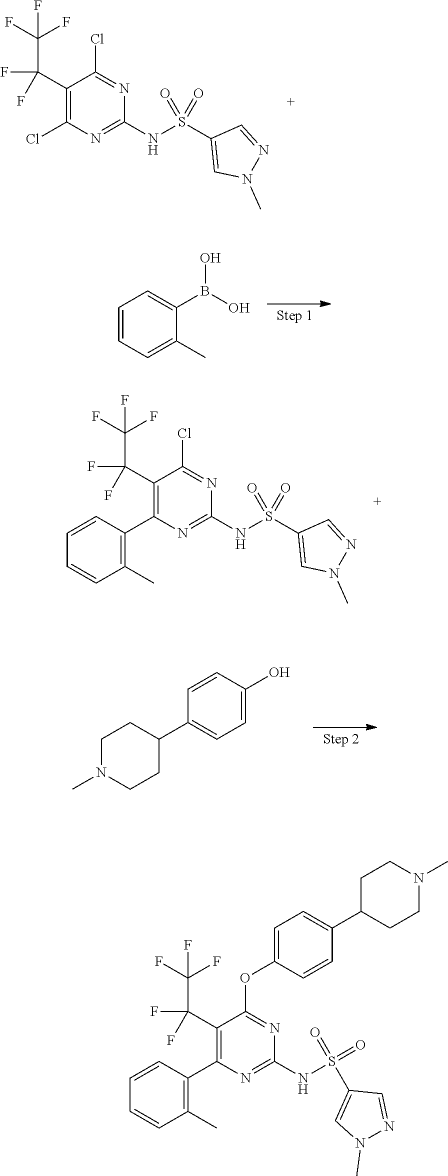

### Step 1: N-[4-chloro-6-(o-tolyl)-5-(1,1,2,2,2-pentafluoroethyl)pyrimidin-2-yl]-1 -methyl-pyrazole-4-sulfonamide

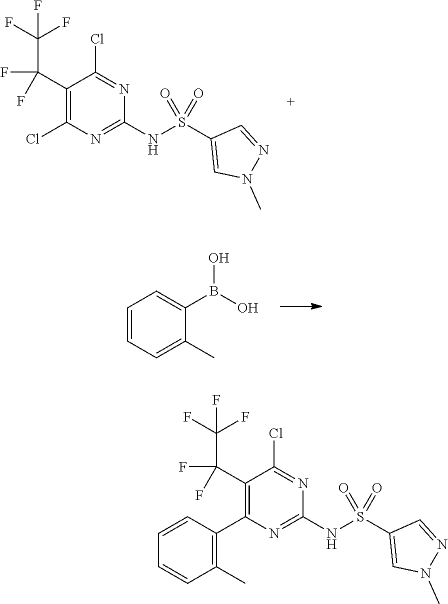

In a 20-mL microwave vial equipped with a magnetic star bar, N-[4,6-dichloro-5-(1,1,2,2,2-pentafluoroethyl)pyrimidin-2-yl]-1-methyl-pyrazole-4-sulfonamide (1.0021 g, 2.187mmol), o-tolylboronic acid (0.3194 g, 2.349 mmol), and Pd(PPh₃)₂C₁₂ (0.055 g, 0.07836 mmol) were placed, and this solid mixture was degassed under three vacuum evacuation/N2 refill sequences. To this mixture, dioxane (10 mL) was added, followed by a solution of K₂CO₃ (1.0011 g, 7.244 mmol) in water (2 mL). This solution was stirred at 90° C. for 20 hours. The reaction mixture was cooled to room temperature, quenched slowly with 1 N HCl solution (10 mL), and extracted with ethyl acetate (3×5 mL). The combined organic extracts were washed with water (10 mL) and saturated aqueous sodium chloride solution (10 mL), then dried over sodium sulfate, filtered, and evaporated in vacuo. This crude product was purified by silica gel chromatography (80 g of silica, 0 to 60% gradient of ethyl acetate/hexanes) to give 0.4168 g of a white foam, which is a ˜1:1 mixture of the desired product N-[4-chloro-6-(o-tolyl)-5-(1,1,2,2,2-pentafluoroethyl)pyrimidin-2-yl]-1-methyl-pyrazole-4-sulfonamide (0.197 g, 19%) ESI-MS m z calc. 481.03986, found 482.2 (M+1)⁺; Retention time: 1.86 minutes (LC method A), and the side product N-[4,6-bis(o-tolyl)-5-(1,1,2,2,2-pentafluoroethyl)pyrimidin-2-yl]-1-methyl-pyrazole-4-sulfonamide (0.22 g, 19%) ESI-MS m/z calc. 537.1258, found 538.3 (M+1)⁺; Retention time: 2.02 minutes. LC method A.

### Step 2: 1-Methyl-N-[4-[4-(1-methyl-4-piperidyl)phenoxy]-6-(o-tolyl)-5-(1,1,2,2,2-pentafluoroethyl)pyrimidin-2-yl]pyrazole-4-sulfonamide

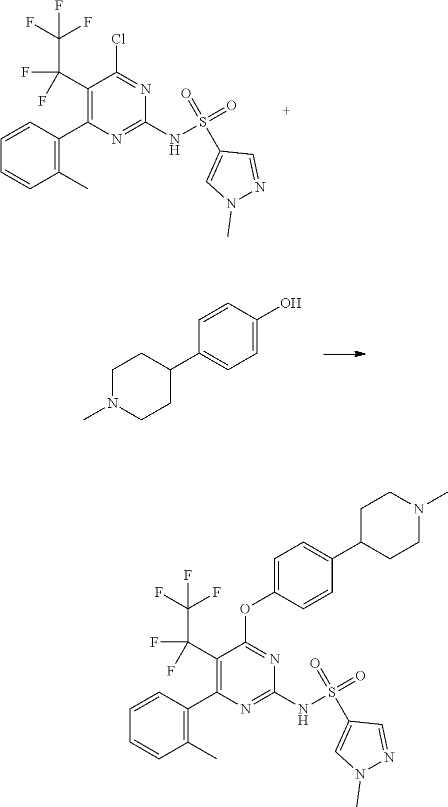 [SMILES: Cc1ccccc1-c1nc(NS(=O)(=O)c2cnn(C)c2)nc(Cl)c1C(F)(F)C(F)(F)F ; CN1CCC(c2ccc(O)cc2)CC1 ; Cc1ccccc1-c1nc(NS(=O)(=O)c2cnn(C)c2)nc(Oc2ccc(C3CCN(C)CC3)cc2)c1C(F)(F)C(F)(F)F]

A mixture of N-[4-chloro-6-(o-tolyl)-5-(1,1,2,2,2-pentafluoroethyl)pyrimidin-2-yl]-1 -methyl-pyrazole-4-sulfonamide (70 mg, 0.1453 mmol), 4-(1-methyl-4-piperidyl)phenol (acetate salt) (56 mg, 0.2228 mmol) and Cs₂CO₃ (160 mg, 0.4911 mmol) in NMP (700 μL) was stirred at 120° C. for 16 hours. The reaction mixture was diluted with MeOH, filtered and purification by HPLC (1-99% ACN in water (HCl modifier)) gave 1-methyl-N-[4-[4-(1-methyl-4-piperidyl)phenoxy]-6-(o-tolyl)-5-(1,1,2,2,2-pentafluoroethyl)pyrimidin-2-yl]pyrazole-4-sulfonamide (hydrochloride salt) (23.7 mg, 24%). ESI-MS m/z calc. 636.1942, found 637.3 (M+1)⁺; Retention time: 1.48 minutes, LC method A.

### Example 379: Preparation of Compound 1354

### Step 1: 1-Methyl-N-[4-[3-(4-methylpiperazin-1-yl)phenoxy]-6-(o-tolyl)-5-(1,1,2,2,2-pentafluoroethyl)pyrimidin-2-yl]pyrazole-4-sulfonamide

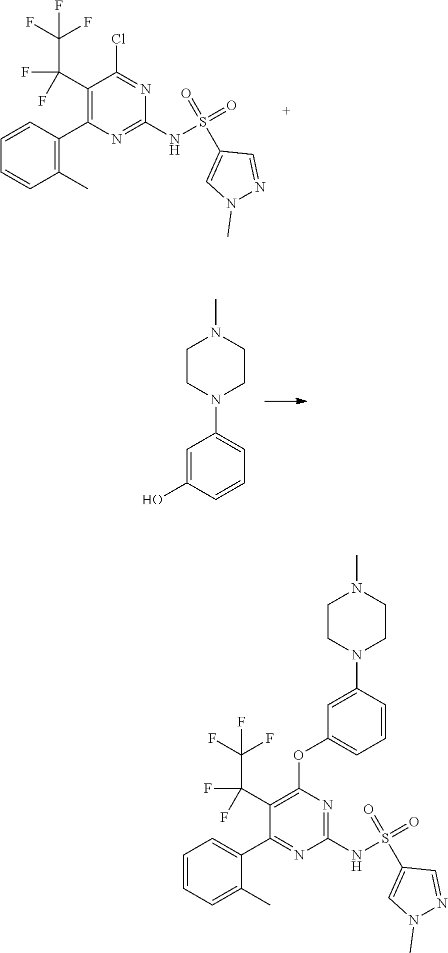 [SMILES: Cc1ccccc1-c1nc(NS(=O)(=O)c2cnn(C)c2)nc(Cl)c1C(F)(F)C(F)(F)F ; CN1CCN(c2cccc(O)c2)CC1 ; Cc1ccccc1-c1nc(NS(=O)(=O)c2cnn(C)c2)nc(Oc2cccc(N3CCN(C)CC3)c2)c1C(F)(F)C(F)(F)F]

A heterogeneous solution of N-[4-chloro-6-(o-tolyl)-5-(1,1,2,2,2-pentafluoroethyl)pyrimidin-2-yl]-1-methyl-pyrazole-4-sulfonamide (12.05 mg, 0.0250 mmol), 3-(4-methylpiperazin-1-yl)phenol, and potassium carbonate in NMP (50.00 μL) was heated in a sealed vial to 115° C. for 16 h. The solution was acidified with acetic acid. The sample was purified by reverse phase HPLC (Waters Sunfire C₁₈ column (100×50 mm, 10 m particle size), gradient: 1-99% acetonitrile in water (5 mM HCl) over 15.0 minutes) to afford 1-methyl-N-[4-[3-(4-methylpiperazin-1-yl)phenoxy]-6-(o-tolyl)-5-(1,1,2,2,2-pentafluoroethyl)pyrimidin-2-yl]pyrazole-4-sulfonamide (hydrochloride salt) as a white solid. ESI-MS m/z calc. 637.18945, found 638.3 (M+1)⁺; Retention time: 1.56 minutes; LC method A.

### Example 380: Preparation of Compound 1406

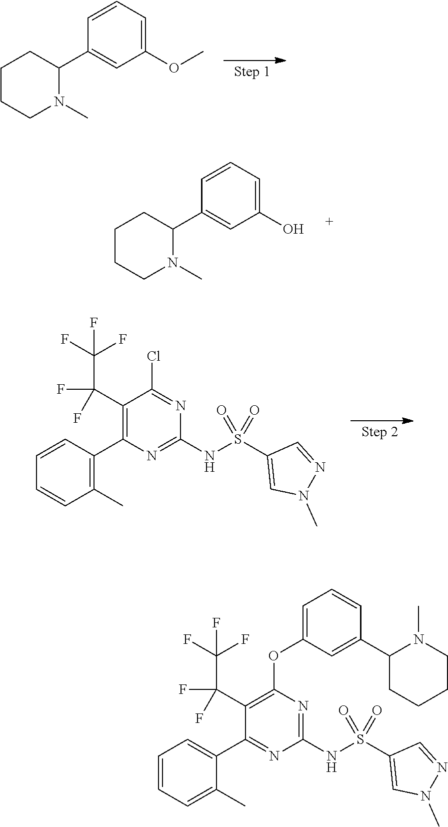 [SMILES: COc1cccc(C2CCCCN2C)c1 ; CN1CCCCC1c1cccc(O)c1 ; Cc1ccccc1-c1nc(NS(=O)(=O)c2cnn(C)c2)nc(Cl)c1C(F)(F)C(F)(F)F ; Cc1ccccc1-c1nc(NS(=O)(=O)c2cnn(C)c2)nc(Oc2cccc(C3CCCCN3C)c2)c1C(F)(F)C(F)(F)F]

### Step 1: 3-(1-Methyl-2-piperidyl)phenol

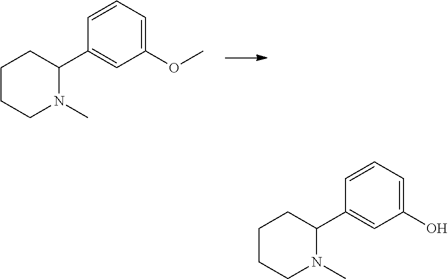

A room temperature solution of 2-(3-methoxyphenyl)piperidine (162.8 mg, 0.8512 mmol) in MeOH (2000 μL) and DCM (3 mL) was treated with formaldehyde (1500 μL, 54mmol)(37% in water), formic acid (1500 μL, 40.0 mmol), and sodium triacetoxyborohydride (322.3 mg, 1.521 mmol). The mixture was stirred for 16 hours at 60° C. and then concentrated in vacuo. The resulting residue was taken up in concentrated hydrobromic acid (1000 μL, 18.42 mmol) and heated at 100° C. for 16 hours and then concentrated in vacuo. The residue was dissolved in DMSO/MeOH (1:1) (1.5 mL) and the solution was filtered and purified by reverse phase chromatography using a 15 minute gradient of 1% MeCN in water to 50% MeCN (HCl modifier) to give 3-(1-methyl-2-piperidyl)phenol (hydrochloride salt) (18.0 mg, 9%) ESI-MS m/z calc. 191.13101, found 192.27 (M+1)⁺; Retention time: 0.54 minutes. LC method A.

### Step 2: 1-Methyl-N-[4-[3-(1-methyl-2-piperidyl)phenoxy]-6-(o-tolyl)-5-(1,1,2,2,2-pentafluoroethyl)pyrimidin-2-yl]pyrazole-4-sulfonamide

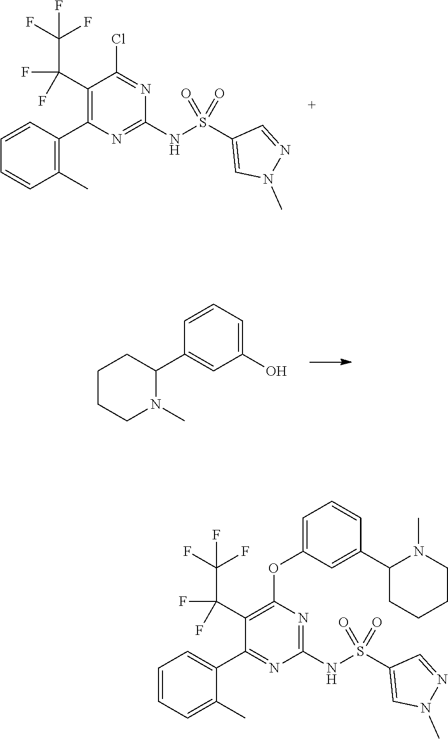 [SMILES: Cc1ccccc1-c1nc(NS(=O)(=O)c2cnn(C)c2)nc(Cl)c1C(F)(F)C(F)(F)F ; CN1CCCCC1c1cccc(O)c1 ; Cc1ccccc1-c1nc(NS(=O)(=O)c2cnn(C)c2)nc(Oc2cccc(C3CCCCN3C)c2)c1C(F)(F)C(F)(F)F]

To a 3-mL vial equipped with a magnetic stir bar, N-[4-chloro-6-(o-tolyl)-5-(1,1,2,2,2-pentafluoroethyl)pyrimidin-2-yl]-1-methyl-pyrazole-4-sulfonamide (16.30 mg, 0.03383 mmol) (16.3 mg, 0.0338 mmol), 3-(1-methyl-2-piperidyl)phenol (hydrochloride salt) (26.17 mg, 0.1149 mmol) (0.115 mmol), K₂CO₃ (21.50 mg, 0.1556 mmol) (21.5 mg, 0.156 mmol) and NMP (500 μL) were added. This slurry was stirred at 120° C. for 13 hours or 37 hours. The reaction mixture was then cooled to room temperature, diluted with MeOH (400 μL), filtered and purified by reverse phase HPLC (1-70% acetonitrile in water using HCl as a modifier) to give the desired product 1-methyl-N-[4-[3-(1-methyl-2-piperidyl)phenoxy]-6-(o-tolyl)-5-(1,1,2,2,2-pentafluoroethyl)pyrimidin-2-yl]pyrazole-4-sulfonamide (hydrochloride salt) (4.2 mg, 18%)). ESI-MS m/z calc. 636.1942, found 637.4 (M+1)⁺; Retention time: 1.52 minutes; LC method A.

### Example 381: Preparation of Compound 1407

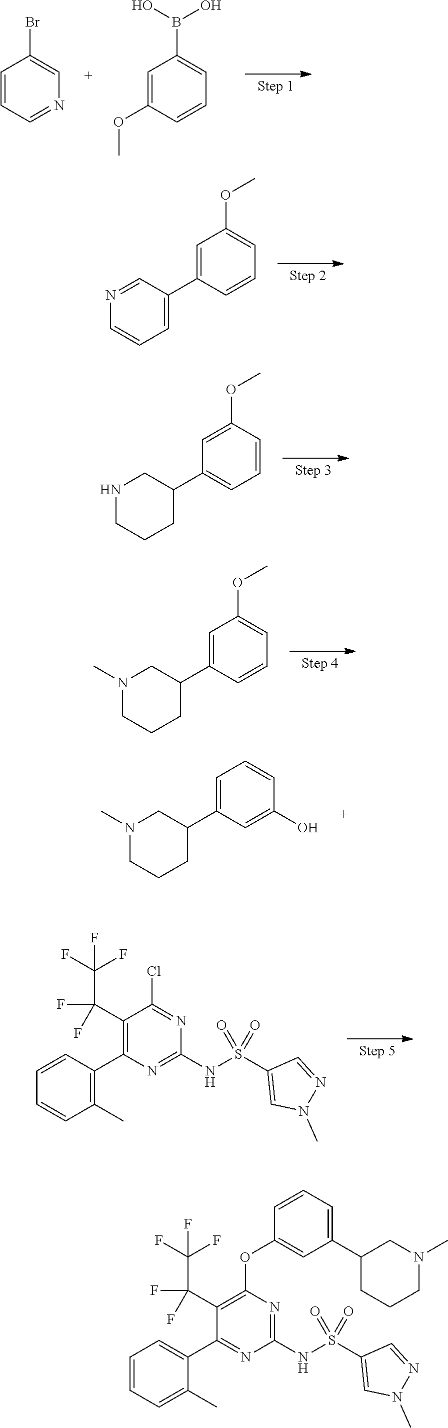

### Step 1: 3-(3-Methoxyphenyl)pyridine

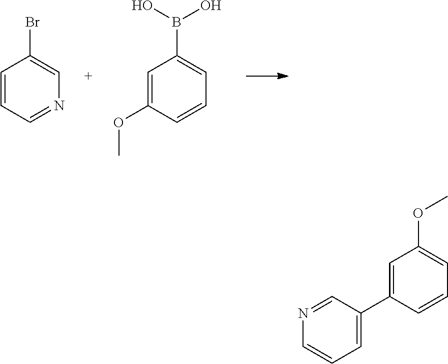

Tetrakis(triphenylphosphine)palladium(0) (0.90 g, 0.779 mmol) was added to a solution of (3-methoxyphenyl)boronic acid (15.00 g, 98.71 mmol), 3-bromopyridine (9.5 mL, 98.61 mmol) and sodium carbonate (20.90 g, 197.2 mmol) in mixture of tetrahydrofuran (200.0 mL), water (100.00 mL) and methanol (50.00 mL). The reaction mixture was bubbled with nitrogen for 5 minutes and heated at reflux for 2 hours followed by stirring at room temperature overnight. The reaction mixture was diluted with water (150 mL) and extracted with EtOAc (3×150 mL). The organic layers were combined, dried over sodium sulfate and concentrated under reduced pressure to afford 3-(3-methoxyphenyl)pyridine (20.58 g, 113%) as brown oil. ¹H NMR (300 MHz, DMSO-d₆) δ ppm 3.82 (s, 3H), 6.95-7.01 (m, 1H), 7.23-7.30 (m, 2H), 7.39 (d, J=7.9 Hz, 1H), 7.42-7.51 (m, 1H), 8.07 (dt, J=7.9, 1.9 Hz, 1H), 8.56 (d, J=4.7 Hz, 1H), 8.89 (d, J=2.1 Hz, 1H). ESI-MS m/z calc. 185.222, found 186.2 (M+1)⁺; Retention time: 1.26 minutes LC method C.

### Step 2: 3-(3-Methoxyphenyl)piperidine

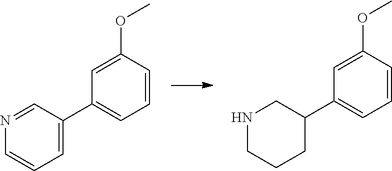

Platinum oxide (2.24 g, 9.864 mmol) was added to a solution of 3-(3-methoxyphenyl)pyridine (18.26 g, 98.69 mmol) in methanol (200 mL) and concentrated HCl (15 mL). Reaction mixture was placed under 50 PSI of hydrogen overnight (2 hours with stirring). The reaction mixture was placed under 50 PSI of hydrogen for 7 more hours. The reaction mixture was filtered over celite, washed with methanol and concentrated under reduced pressure to afford crude 3-(3-methoxyphenyl)piperidine (hydrochloride salt) (29.66 g, 132%) as a yellow oil. ESI-MS m/z calc. 191.269, found 192.2 (M+1)⁺; Retention time: 1.08 minutes. (LC method C).

### Step 3: 3-(3-Methoxyphenyl)-1-methyl-piperidine

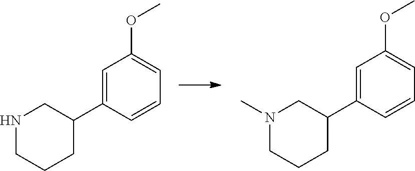

Formaldehyde (11.0 mL of 37% in water, 148.2 mmol), sodium triacetoxyborohydride (41.9 g, 197.7 mmol) and acetic acid (11.30 mL, 197.6 mmol) were added to a solution of 3-(3-methoxyphenyl)piperidine (hydrochloride salt) (22.5 g, 98.80 mmol) in dichloromethane (450 mL) at 0° C. and reaction mixture was stirred at room temperature overnight. The reaction mixture was basified using 5% aqueous sodium bicarbonate (until pH 7-8) and the resulting mixture was extracted with dichloromethane (3×300 mL). Organic layers were combined, dried over sodium sulfate and concentrated under reduced pressure. The residue was purified by silica gel chromatography using 0% to 10% of methanol (containing 1% of ammonium hydroxide) in dichloromethane to afford 3-(3-methoxyphenyl)-1-methyl-piperidine (6.99 g, 34%) as orange oil. ¹H NMR (300 MHz, CDCl₃) δ ppm 1.33-1.51 (m, 1H), 1.69-1.85 (m, 2H), 1.87-2.05 (m, 3H), 2.31 (s, 3H), 2.73-3.04 (m, 3H), 3.80 (s, 3H), 6.71-6.88 (m, 3H), 7.17-7.26 (m, 1H). ESI-MS m/z calc. 205.296, found 206.2 (M+1)⁺; Retention time: 1.1 minutes. LC method C.

### Step 4: 3-(1-Methyl-3-piperidyl)phenol

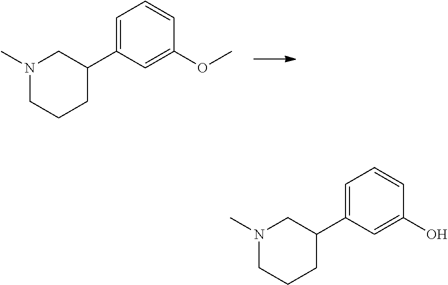

A boron tribromide solution (4.9 mL of 1 M, 4.900 mmol) was added to a cold solution (−78° C.) of 3-(3-methoxyphenyl)-1-methyl-piperidine (500 mg, 2.436 mmol) in dichloromethane (5 mL) under nitrogen. The reaction mixture was allowed to warm and stirred at room temperature overnight. The reaction mixture was quenched with water (5 mL) and saturated aqueous sodium carbonate was added until pH 10. The resulting mixture was extracted using ethyl acetate (3×10 mL). The organic layers were combined, dried over sodium sulfate and concentrated under reduced pressure (690 mg of off-white foamy). The residue was diluted in methanol (5 mL) and treated with 2 N HCl in diethyl ether (2 mL). The solution was concentrated under reduced pressure. The residue was triturated with diethyl ether for 30 minutes, filtered and the residue was freeze dried overnight. The residue was diluted in dichloromethane and precipitated with diethyl ether without any success. The residue was diluted with methanol and concentrated under reduced pressure to afford 3-(1-methyl-3-piperidyl)phenol (hydrochloride salt) (300 mg, 43% yield) as beige solid. ¹H NMR (300 MHz, methanol-d₄) δ ppm 1.64-2.15 (m, 3H), 2.15-2.32 (m, 1H), 2.87 (s, 3H), 2.95-3.15 (m, 1H), 3.17-3.27 (m, 1H), 3.40-3.63 (m, 2H), 3.84-4.02 (m, 1H), 4.15-4.28 (m, 1H), 6.63-6.89 (m, 3H), 7.09-7.25 (m, 1H). ESI-MS m/z calc. 191.269, found 192.2 (M+1)⁺; Retention time: 0.54 minutes, LC method C.

### Step 5: 1-Methyl-N-[4-[3-(1-methyl-3-piperidyl)phenoxy]-6-(o-tolyl)-5-(1,1,2,2,2-pentafluoroethyl)pyrimidin-2-yl]pyrazole-4-sulfonamide

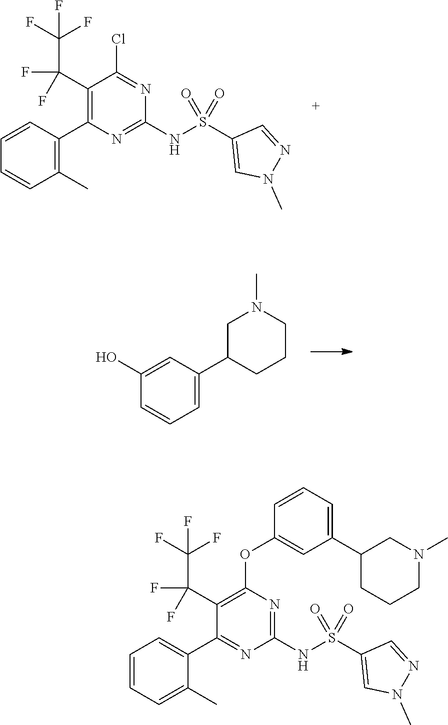 [SMILES: Cc1ccccc1-c1nc(NS(=O)(=O)c2cnn(C)c2)nc(Cl)c1C(F)(F)C(F)(F)F ; CN1CCCC(c2cccc(O)c2)C1 ; Cc1ccccc1-c1nc(NS(=O)(=O)c2cnn(C)c2)nc(Oc2cccc(C3CCCN(C)C3)c2)c1C(F)(F)C(F)(F)F]

To a 3-mL vial equipped with a magnetic stir bar, N-[4-chloro-6-(o-tolyl)-5-(1,1,2,2,2-pentafluoroethyl)pyrimidin-2-yl]-1-methyl-pyrazole-4-sulfonamide (16.30 mg, 0.03383 mmol) (16.3 mg, 0.0338 mmol), 3-(1-methyl-3-piperidyl)phenol (hydrochloride salt) (36.67 mg, 0.1149 mmol) (0.115 mmol), K₂CO₃ (21.50 mg, 0.1556 mmol) (21.5 mg, 0.156 mmol) and NMP (500 μL) were added. This slurry was stirred at 120° C. for 13 hours or 37 hours. The reaction mixture was then cooled to room temperature, diluted with MeOH (400 μL), filtered and purified by reverse phase HPLC (1-70% acetonitrile in water using HCl as a modifier) to give the desired product 1-methyl-N-[4-[3-(1-methyl-3-piperidyl)phenoxy]-6-(o-tolyl)-5-(1,1,2,2,2-pentafluoroethyl)pyrimidin-2-yl]pyrazole-4-sulfonamide (hydrochloride salt) (4.3 mg, 19%). ESI-MS m/z calc. 636.1942, found 637.4 (M+1)⁺; Retention time: 1.54 minutes; LC method A.

### Example 382: Preparation of Compound 1408

### Step 1: 1-Methyl-N-[4-[3-(1-methyl-4-piperidyl)phenoxy]-6-(o-tolyl)-5-(1,1,2,2,2-pentafluoroethyl)pyrimidin-2-yl]pyrazole-4-sulfonamide

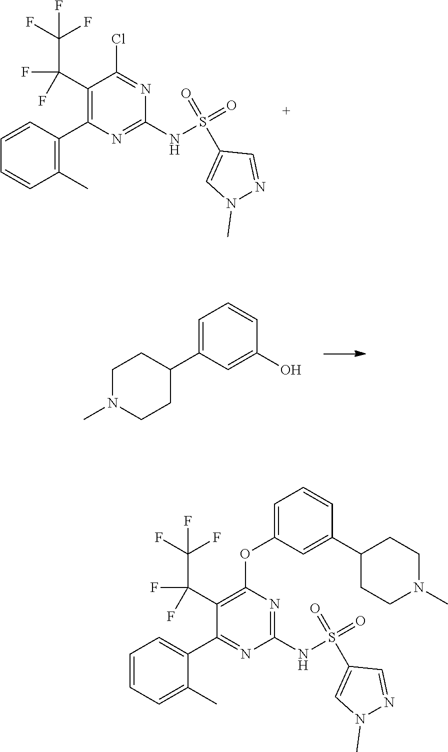 [SMILES: Cc1ccccc1-c1nc(NS(=O)(=O)c2cnn(C)c2)nc(Cl)c1C(F)(F)C(F)(F)F ; CN1CCC(c2cccc(O)c2)CC1 ; Cc1ccccc1-c1nc(NS(=O)(=O)c2cnn(C)c2)nc(Oc2cccc(C3CCN(C)CC3)c2)c1C(F)(F)C(F)(F)F]

The compound was prepared in a manner analogous to that described above using 3-(1-methyl-4-piperidyl)phenol to give 1-methyl-N-[4-[3-(1-methyl-4-piperidyl)phenoxy]-6-(o-tolyl)-5-(1,1,2,2,2-pentafluoroethyl)pyrimidin-2-yl]pyrazole-4-sulfonamide (hydrochloride salt) (7.1 mg, 31%). ESI-MS m/z calc. 636.1942, found 637.4 (M+1)⁺; Retention time: 1.54 minutes; LC method A.

### Example 383: Preparation of Compound 1409

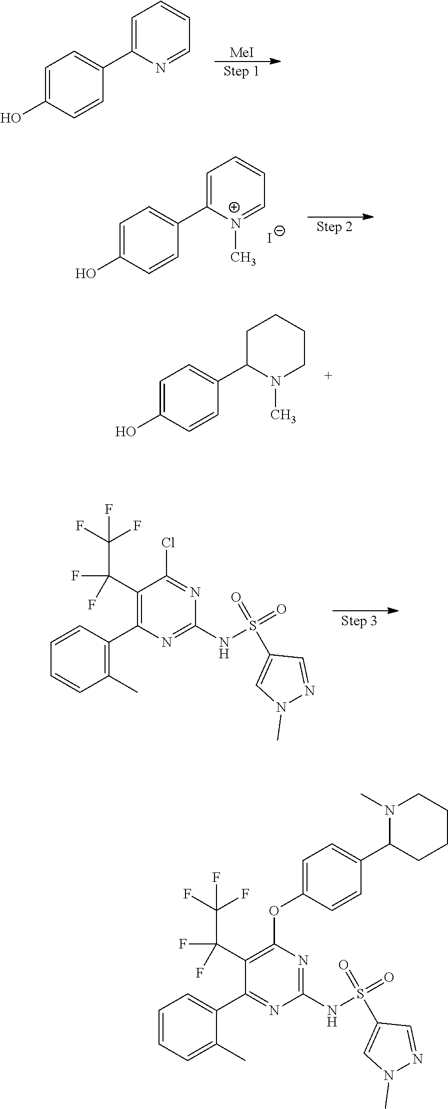

### Step 1: 2-(4-Hydroxyphenyl)-1-methylpyridin-1-ium iodide

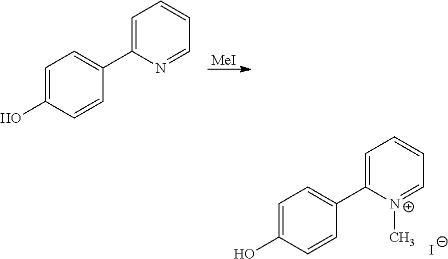

A solution of 4-(pyridin-2-yl)phenol (3.5 g, 20.47 mmol) in acetone (20 mL) and iodomethane (20 mL) was stirred at reflux (60° C.) for 16 hours. The mixture was cooled and filtered, and the solid pink cake was rinsed with acetone, ethyl acetate and hexanes to afford the desired 2-(4-hydroxyphenyl)-1-methylpyridin-1-ium iodide (6.4 g, 99.8% yield) as a pink solid. ESI-MS m/z: calc.186.09, found 186.1, Retention time: 1.19 minutes.

### Step 2: 4-(1-Methylpiperidin-2-yl)phenol

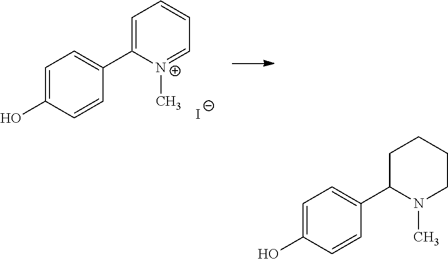

To a solution of 2-(4-hydroxyphenyl)-1-methylpyridin-1-ium iodide (4.5 g, 14.37 mmol) in methanol (110 mL) was added Raney-nickel (3 large spatula tips). The mixture was shaken in a Parr shaker at 60 psi of hydrogen for 20 hours, at which time it was carefully filtered and concentrated to dryness. The residue was treated with 0.5 N HCl (150 mL) and ethyl acetate (100 mL). The aqueous (pH-1) layer was neutralized by the addition of 2 N sodium hydroxide to pH-8-9, and then extracted with a mixture of chloroform and isopropanol (3:1, volume:volume, 3×200 mL). The organic layer was washed with brine, dried over sodium sulfate and concentrated to afford 4-(1-methyl-2-piperidyl)phenol (2.62 g, 95% yield) as an off-white solid. ESI-MS m/z calc.191.131, found 192.4 (M+1)⁺. Retention time: 0.74 minutes. ¹H NMR (CDCl₃, 250 MHz) δ 7.19 (d, J=8.25 Hz, 2H), 6.76 (d, J=8.75 Hz, 2H), 3.20-3.05 (m, 1H), 2.90-2.78 (m, 1H), 2.28-2.10 (m, 1H), 2.06 (s, 3H), 1.90-1.65 (m, 5H), 1.50-1.30 (m, 1H).

### Step 3: 1-Methyl-N-[4-[4-(1-methyl-2-piperidyl)phenoxy]-6-(o-tolyl)-5-(1,1,2,2,2-pentafluoroethyl)pyrimidin-2-yl]pyrazole-4-sulfonamide

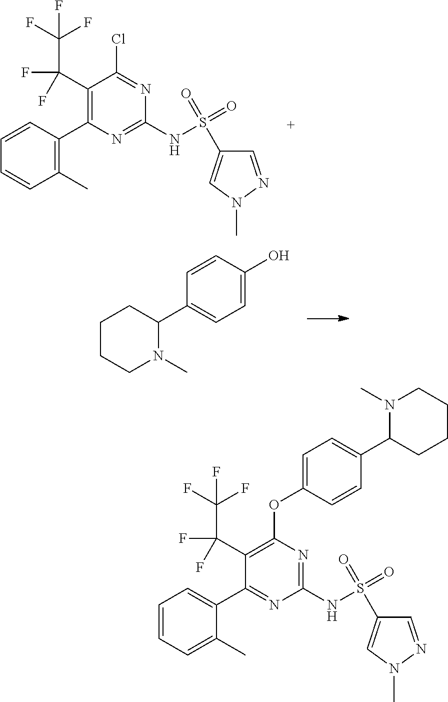

The compound was prepared in a manner analogous to that described above using 4-(1-methyl-2-piperidyl)phenol to give the desired product 1-methyl-N-[4-[4-(1-methyl-2-piperidyl)phenoxy]-6-(o-tolyl)-5-(1,1,2,2,2-pentafluoroethyl)pyrimidin-2-yl]pyrazole-4-sulfonamide (hydrochloride salt) (5.3 mg, 23%). ESI-MS m/z calc. 636.1942, found 637.4 (M+1)⁺; Retention time: 1.52 minutes; LC method A. ¹H NMR (400 MHz, dimethylsulfoxide-d₆) δ 12.73-12.01 (br s, 1H, D20 exchangeable), 10.67-10.34 (br s, 1H, D20 exchangeable), 7.79 (d, J=6.5 Hz, 2H), 7.72 (d, J=2.1 Hz, 1H), 7.45 (d, J=8.8 Hz, 2H), 7.37 (td, J=7.4, 1.3 Hz, 1H), 7.31 (d, J=7.3 Hz, 1H), 7.26 (t, J=7.4 Hz, 1H), 7.16 (d, J=7.6 Hz, 1H), 6.96 (s, 1H), 4.36-4.24 (m, 1H), 3.78 (s, 3H), 3.62-3.54 (m, 1H), 3.19-3.03 (m, 1H), 2.48 (d, J=3.8 Hz, 3H), 2.13-1.83 (m, 5H), 2.09 (s, 3H), 1.68-1.54 (m, 1H).

### Example 384: Preparation of Compound 1410

### Step 1: 3-(Pyridin-3-yl)phenol

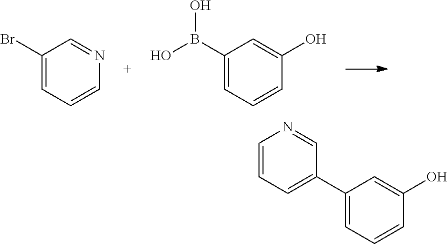

In 20 mL microwave vial, a mixture of 3-bromopyridine (960 mg, 6 mmol), 3-hydroxyphenyl boronic acid (912 mg, 6.6 mol), [1,1′-bis(diphenylphosphino)ferrocene]palladium(II) dichloride (44 mg, 0.6 μmol), 2 M aqueous potassium carbonate solution (4.5 mL, 9 mmol) in acetonitrile (13.5 mL) was irradiated at 135° C. for 12 minutes. Eleven batches were run, and the reaction mixtures were combined and filtered through a pad of Celite. The mother liquor was acidified by 1 M hydrogen chloride to pH 4 and neutralized by solid sodium hydrogen carbonate. All solvents were concentrated in vacuum; the residue was taken in chloroform (300 mL), dried over anhydrous magnesium sulfate and concentrated in vacuum to give 3-(pyridin-3-yl)phenol (10.2 g, 90.3%) as an off-white solid. ESI-MS m/z calc. 171.2, found 172.9 (M+1). Retention time: 2.00 minutes.

### Step 2: 3-(3-Hydroxyphenyl)-1-methylpyridin-1-ium iodide

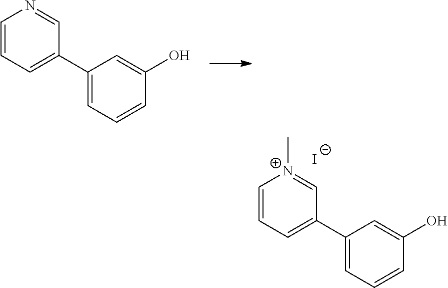

3-(Pyridin-3-yl)phenol (9.6 g, 56 mmol) was dissolved in tetrahydrofuran (120 mL) and methyl iodide (11.9 g, 84.1 mmol) was added. The reaction mixture was stirred at 60° C. for 16 hours. The formed precipitate was filtered off, washed with in tetrahydrofuran (20 mL) to give 3-(3-hydroxyphenyl)-1-methylpyridin-1-ium iodide (15.5 g, 88.4%) as an off-white solid. ESI-MS m/z calc. 186.1, found 185.8 (M+1). Retention time: 2.44 minutes.

### Step 3: 3-(3-Hydroxyphenyl)-1-methylpiperidin-1-ium iodide

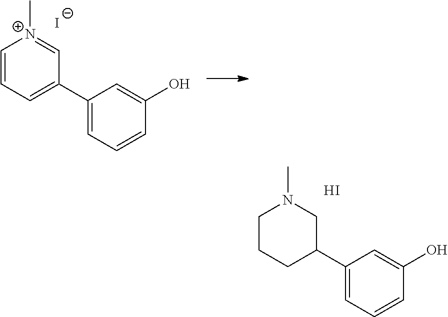

3-(3-Hydroxyphenyl)-1-methylpyridin-1-ium iodide (15.5 g, 49.5 mmol) was dissolved in a mixture of methanol (450 ml) and water (65 mL). Approximately 5 g of nickel (Raney) was added and the mixture was hydrogenated in a Parr shaker at 60 psi for 48 hours. Fresh 5 g portions of Raney nickel were added after 16 hours and 32 hours. The reaction mixture was filtered, and the filtrate was concentrated in vacuum to give 3-(3-hydroxyphenyl)-1-methylpiperidin-1-ium iodide (13.5 g, 85.5%) as a pale brown solid. ESI-MS m/z calc. 191.13, found 192.1 (M+1). Retention time: 6.68 minutes. ¹H NMR (250 MHz, DMSO-d₆) δ (ppm): 9.39 (s, 1H), 7.15 (t, J=2.8 Hz, 1H), 6.66 (t, J=2.8 Hz, 2H), 6.64 (s, 1H), 3.28 (br. s, 2H), 2.82-2.5.8 (m, 3H), 2.66 (s, 3H), 1.91-1.50 (m, 4H).

### Step 4: 1-Methyl-N-[4-[4-(1-methyl-3-piperidyl)phenoxy]-6-(o-tolyl)-5-(1,1,2,2,2-pentafluoroethyl)pyrimidin-2-yl]pyrazole-4-sulfonamide

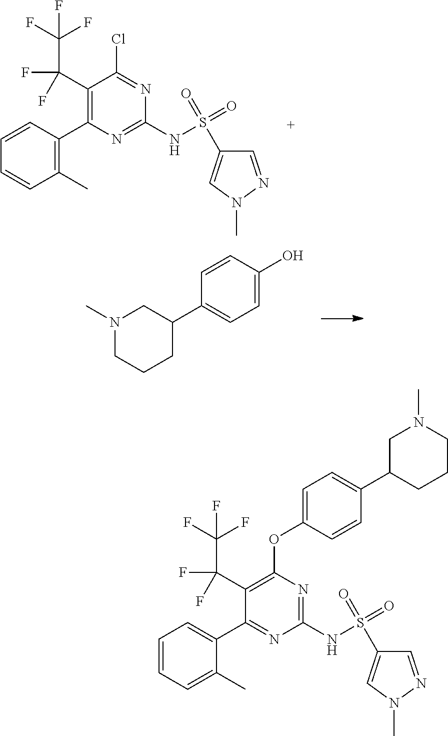

To a 3-mL vial equipped with a magnetic stir bar, N-[4-chloro-6-(o-tolyl)-5-(1,1,2,2,2-pentafluoroethyl)pyrimidin-2-yl]-1-methyl-pyrazole-4-sulfonamide (16.30 mg, 0.03383 mmol) (16.3 mg, 0.0338 mmol), 4-(1-methyl-3-piperidyl)phenol (hydroiodic salt) (36.67 mg, 0.1149 mmol) (0.115 mmol), K₂CO₃ (21.50 mg, 0.1556 mmol) (21.5 mg, 0.156 mmol) and NMP (500 μL) were added. This slurry was stirred at 120° C. for 13 hours or 37 hours. The reaction mixture was then cooled to room temperature, diluted with MeOH (400 μL), filtered and purified by reverse phase HPLC (1-70% acetonitrile in water using HCl as a modifier) to give the desired product 1-methyl-N-[4-[4-(1-methyl-3-piperidyl)phenoxy]-6-(o-tolyl)-5-(1,1,2,2,2-pentafluoroethyl)pyrimidin-2-yl]pyrazole-4-sulfonamide (hydrochloride salt) (7.8 mg, 34%). ESI-MS m/z calc. 636.1942, found 637.4 (M+1)⁺; Retention time: 1.54 minutes; LC method A.

### Example 385: Preparation of Compound 1411

### Step 1: 1-Methyl-N-[4-[4-(4-methylpiperazin-1-yl)phenoxy]-6-(o-tolyl)-5-(1,1,2,2,2-pentafluoroethyl)pyrimidin-2-yl]pyrazole-4-sulfonamide

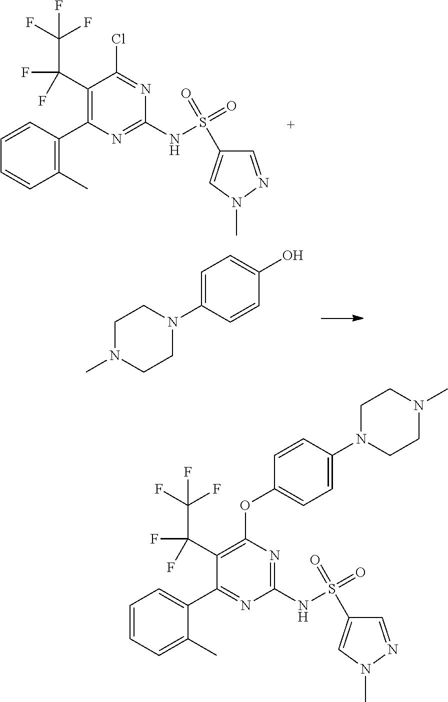

The compound was prepared in a manner analogous to that described above using 4-(4-methylpiperazin-1-yl)phenol to give 1-methyl-N-[4-[4-(4-methylpiperazin-1-yl)phenoxy]-6-(o-tolyl)-5-(1,1,2,2,2-pentafluoroethyl)pyrimidin-2-yl]pyrazole-4-sulfonamide (hydrochloride salt) (6.1 mg, 27%). ESI-MS m/z calc. 637.18945, found 638.4 (M+1)⁺; Retention time: 1.49 minutes; LC method A. ¹H NMR (400 MHz, dimethylsulfoxide-d₆) δ 12.44-12.12 (bs, 1H, D20 exchangeable), 10.13-9.93 (bs, 1H, D20 exchangeable), 7.51 (s, 1H), 7.35 (td, J=7.4, 1.4 Hz, 1H), 7.30 (d, J=7.4 Hz, 1H), 7.28-7.11 (m, 6H), 7.08 (s, 1H), 3.93-3.83 (m, 2H), 3.74 (s, 3H), 3.58-3.49 (m, 2H), 3.24-3.13 (m, 2H), 3.09-2.99 (m, 2H), 2.85 (d, J=4.0 Hz, 3H), 2.07 (s, 3H)

### Example 386: Preparation of Compound 1412

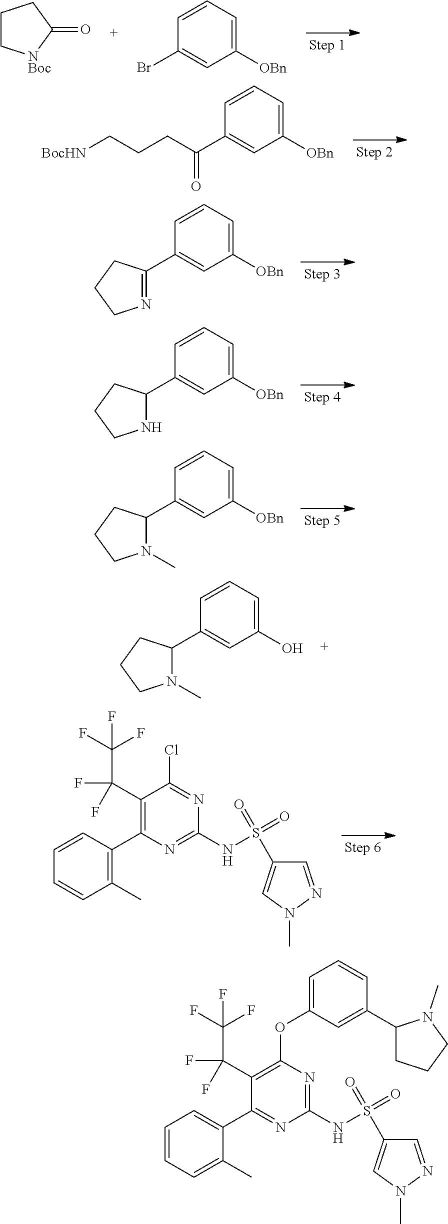

### Step 1 [4-(3-Benzyloxy-phenyl)-4-oxo-butyl]-carbamic acid tert-butyl ester

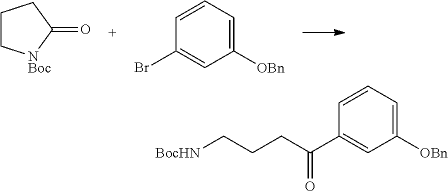 [SMILES: CC(C)(C)OC(=O)N1CCCC1=O ; Brc1cccc(OCc2ccccc2)c1 ; CC(C)(C)OC(=O)NCCCC(=O)c1cccc(OCc2ccccc2)c1]

Benzyloxy-3-bromo-benzene (7.75 g, 29.45 mmol) was dissolved in THF (100 mL) and cooled in a dry ice acetone bath at −70° C. under nitrogen. nBuLi (15.4 mL, 2.5 M, 38.5 mmol) was added via syringe over 5 minutes. The mixture was stirred at this temperature for 30 minutes. 2-Oxo-pyrrolidine-1-carboxylic acid tert-butyl ester (6 g, 32.43 mmol) in THE (10 mL) was added. The reaction mixture was stirred at −78° C. for 1 hour and then slowly warmed up to 0° C. and quenched by adding NH₄C₁ (sat. aq. ˜ 10 mL). The mixture was concentrated to remove most volatiles. The residue was partitioned between EtOAc and water (˜50 mL each). The organic layer was washed with brine, dried over Na₂SO₄, filtered and concentrated. The residue was purified by silica gel chromatography, using 0-30% EtOAc in hexanes to afford [4-(3-Benzyloxy-phenyl)-4-oxo-butyl]-carbamic acid tert-butyl ester (6.75 g, 18.28 mmol, 62% yield) as oil. ESI-MS m/z calc. 369.19, found 370.4 (M+1)⁺; Retention time: 3.80 minutes.

### Step 2: 5-(3-Benzyloxy-phenyl)-3,4-dihydro-2H-pyrrole

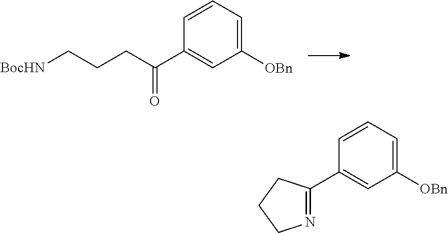 [SMILES: CC(C)(C)OC(=O)NCCCC(=O)c1cccc(OCc2ccccc2)c1 ; c1ccc(COc2cccc(C3=NCCC3)c2)cc1]

[4-(3-Benzyloxy-phenyl)-4-oxo-butyl]-carbamic acid tert-butyl ester (6.75 g, 18.28 mmol) was dissolved in DCM (20 mL) at rt. TFA (˜20 mL) was added. The mixture was stirred for 2 hours. It was then concentrated to dryness. The crude was taken in DCM (30 mL) and washed with sat. aq. NaHCO₃(20 mL). The DCM layer was separated, dried over Na₂SO₄, filtered and concentrated to afford crude 5-(3-Benzyloxy-phenyl)-3,4-dihydro-2H-pyrrole (5 g, 19.8 mmol, 108% yield). This material was used in the next step without further purification. ESI-MS m/z calc. 251.13, found 252.2 (M+1)⁺; Retention time: 2.36 minutes

### Step 3: 2-(3-Benzyloxy-phenyl)-pyrrolidine

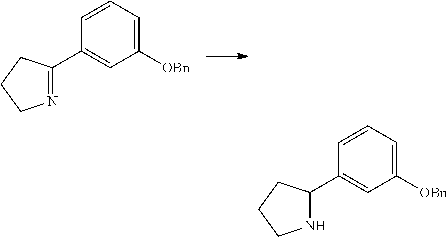 [SMILES: c1ccc(COc2cccc(C3=NCCC3)c2)cc1 ; c1ccc(COc2cccc(C3CCCN3)c2)cc1]

Crude 5-(3-Benzyloxy-phenyl)-3,4-dihydro-2H-pyrrole (5 g, 19.8 mmol) was dissolved in EtOH (40 mL) and cooled in an ice water bath. NaBH₄ (718 mg, 19 mmol) was added in small portions. The mixture was stirred 1 hour after complete addition. NH₄C₁ (sat. aq.) was added to quench the reaction. The mixture was concentrated, and the residue was taken into DCM (˜40 mL) and washed with brine. The layers were separated, and the DCM solution was dried over Na₂SO₄, filtered and concentrated. This crude 2-(3-Benzyloxy-phenyl)-pyrrolidine (˜5 g, 99% yield) was used in the next step without further purification. ESI-MS m/z calc. 253.15, found 254.0 (M+1)⁺; Retention time: 2.41 minutes

### Step 4: 2-(3-Benzyloxy-phenyl)-1-methyl-pyrrolidine

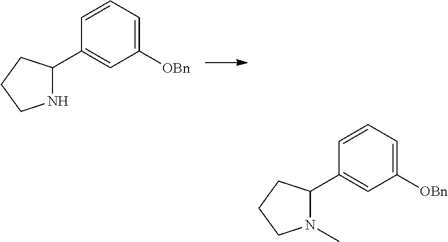 [SMILES: c1ccc(COc2cccc(C3CCCN3)c2)cc1 ; CN1CCCC1c1cccc(OCc2ccccc2)c1]

Crude 2-(3-Benzyloxy-phenyl)-pyrrolidine (˜ 5 g, 19 mmol assumed) was dissolved in THE (40 mL), formaldehyde (8 mL, 98.5 mmol, 37% in water) was added, followed by sodium triacetoxyborohydride (4.2 g, 19.8 mmol) and 3 drops of AcOH. The mixture was stirred at room temperature for 2 hours. It was then concentrated under vacuum to remove most of the THF. The residue was partitioned between DCM and an aqueous saturated NaHCO₃solution (40mL each). The layers were separated, and the DCM solution was washed with water (40 mL), brine, dried over Na₂SO₄, filtered and concentrated to afford crude 2-(3-Benzyloxy-phenyl)-1 -methyl-pyrrolidine (4.6 g, 17.16 mmol, 90% yield). ESI-MS m/z calc. 267.16, found 268.1 (M+1)⁺; Retention time: 2.47 minutes.

### Step 5: 3-(1-Methyl-pyrrolidin-2-yl)-phenol

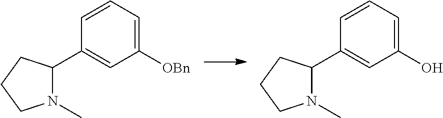 [SMILES: CN1CCCC1c1cccc(OCc2ccccc2)c1 ; CN1CCCC1c1cccc(O)c1]

2-(3-Benzyloxy-phenyl)-1-methyl-pyrrolidine (4.6 g, 17.16 mmol) was dissolved in MeOH (40 mL) in a pressure flask. The solution was degassed and refilled with nitrogen. Pd/C (100 mg, 10% dry basis) and Pd(OH)₂/C (100 mg, 20% dry basis) were added. The mixture was placed under 50 psi of hydrogen pressure in a Parr shaker for 3 hours. The mixture was purged with nitrogen and filtered through a Celite pad and washed with MeOH. The filtrate was concentrated and the residue was purified by reversed phase prep HPLC using 0 to 100% water-acetonitrile (buffered with 0.1% hydrochloric acid) to afford 3-(1-Methyl-pyrrolidin-2-yl)-phenol (hydrochloride salt) (2.2 g, 10.30 mmol, 60% yield) as a white solid. ESI-MS m/z calc. 177.12, found 178.1 (M+1)⁺; Retention time: 0.69 minutes. ¹H NMR (250 MHz, DMSO-d₆) δ (ppm) 7.09 (t, J=7.75 Hz, 1H), 6.7-6.8 (m, 2H), 6.615 (dd, J=2.25, 1 Hz, 1H), 3.135 (t, J=7.5 Hz, 1H), 2.964 (t, J=8.5 Hz, 1H), 2.3-2.0 (m,1 H), 2.05 (s, 3H), 1.45-1.95 (m, 4H).

### Step 6: 1-Methyl-N-[4-[3-(1-methylpyrrolidin-2-yl)phenoxy]-6-(o-tolyl)-5-(1,1,2,2,2-pentafluoroethyl)pyrimidin-2-yl]pyrazole-4-sulfonamide

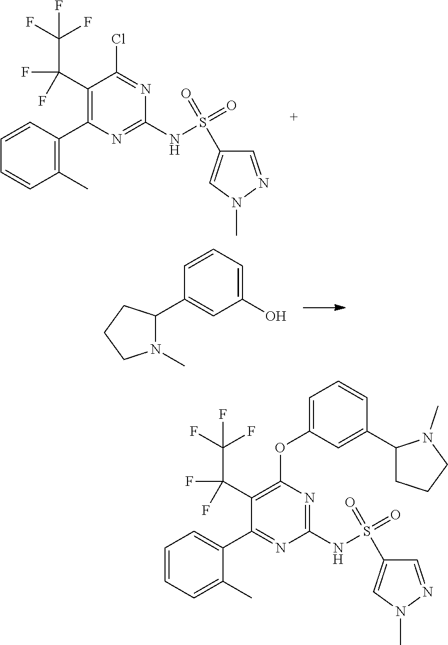

The compound was prepared in a manner analogous to that described above using 3-(1-methylpyrrolidin-2-yl)phenol to give 1-methyl-N-[4-[3-(1-methylpyrrolidin-2-yl)phenoxy]-6-(o-tolyl)-5-(1,1,2,2,2-pentafluoroethyl)pyrimidin-2-yl]pyrazole-4-sulfonamide (hydrochloride salt) (2.2 mg, 10%). ESI-MS m/z calc. 622.1785, found 623.4 (M+1)⁺; Retention time: 1.48 minutes; LC method A.

### Example 387: Preparation of Compound 1413

### Step 1: N-[4-[2-fluoro-3-(1-methyl-4-piperidyl)phenoxy]-6-(o-tolyl)-5-(1,1,2,2,2-pentafluoroethyl)pyrimidin-2-yl]-1-methyl-pyrazole-4-sulfonamide

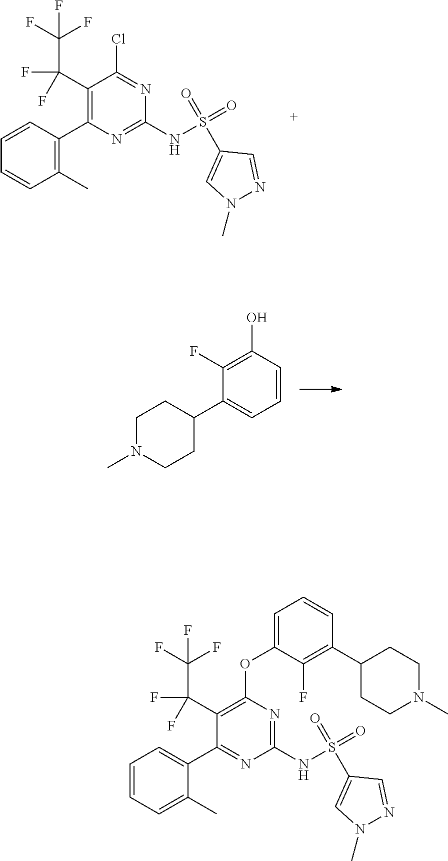 [SMILES: Cc1ccccc1-c1nc(NS(=O)(=O)c2cnn(C)c2)nc(Cl)c1C(F)(F)C(F)(F)F ; CN1CCC(c2cccc(O)c2F)CC1 ; Cc1ccccc1-c1nc(NS(=O)(=O)c2cnn(C)c2)nc(Oc2cccc(C3CCN(C)CC3)c2F)c1C(F)(F)C(F)(F)F]

The compound was prepared in a manner analogous to that described above using 2-fluoro-3-(1-methyl-4-piperidyl)phenol to give N-[4-[2-fluoro-3-(1-methyl-4-piperidyl)phenoxy]-6-(o-tolyl)-5-(1,1,2,2,2-pentafluoroethyl)pyrimidin-2-yl]-1-methyl-pyrazole-4-sulfonamide (hydrochloride salt) (8.3 mg, 36%). ESI-MS m/z calc. 654.18475, found 655.4 (M+1)⁺; Retention time: 1.54 minutes; LC method A.

### Example 388: Preparation of Compound 1414

### Step 1: N-[4-[2-fluoro-5-(1-methyl-4-piperidyl)phenoxy]-6-(o-tolyl)-5-(1,1, 2,2, 2-pentafluoroethyl)pyrimidin-2-yl]-1-methyl-pyrazole-4-sulfonamide

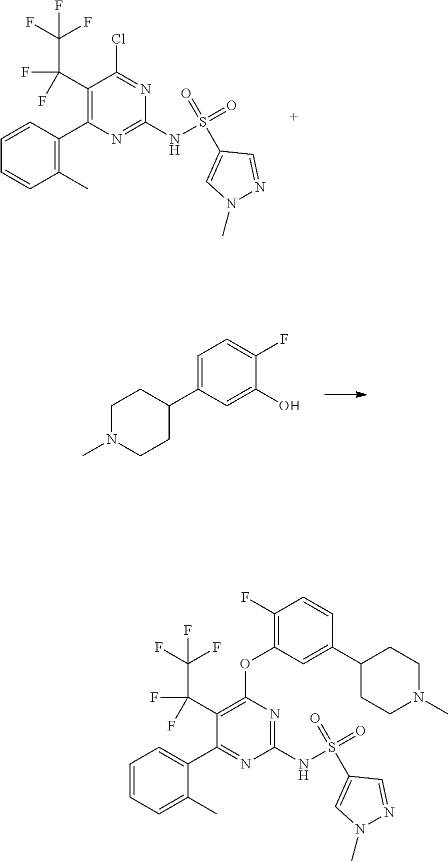 [SMILES: Cc1ccccc1-c1nc(NS(=O)(=O)c2cnn(C)c2)nc(Cl)c1C(F)(F)C(F)(F)F ; CN1CCC(c2ccc(F)c(O)c2)CC1 ; Cc1ccccc1-c1nc(NS(=O)(=O)c2cnn(C)c2)nc(Oc2cc(C3CCN(C)CC3)ccc2F)c1C(F)(F)C(F)(F)F]

The compound was prepared in a manner analogous to that described above using N-[4-[2-fluoro-5-(1-methyl-4-piperidyl)phenoxy]-6-(o-tolyl)-5-(1,1,2,2,2-pentafluoroethyl)pyrimidin-2-yl]-1-methyl-pyrazole-4-sulfonamide (hydrochloride salt) (8.4 mg, 36%). ESI-MS m/z calc. 654.18475, found 655.4 (M+1)⁺; Retention time: 1.54 minutes; LC method A.

### Example 389: Preparation of Compound 1415

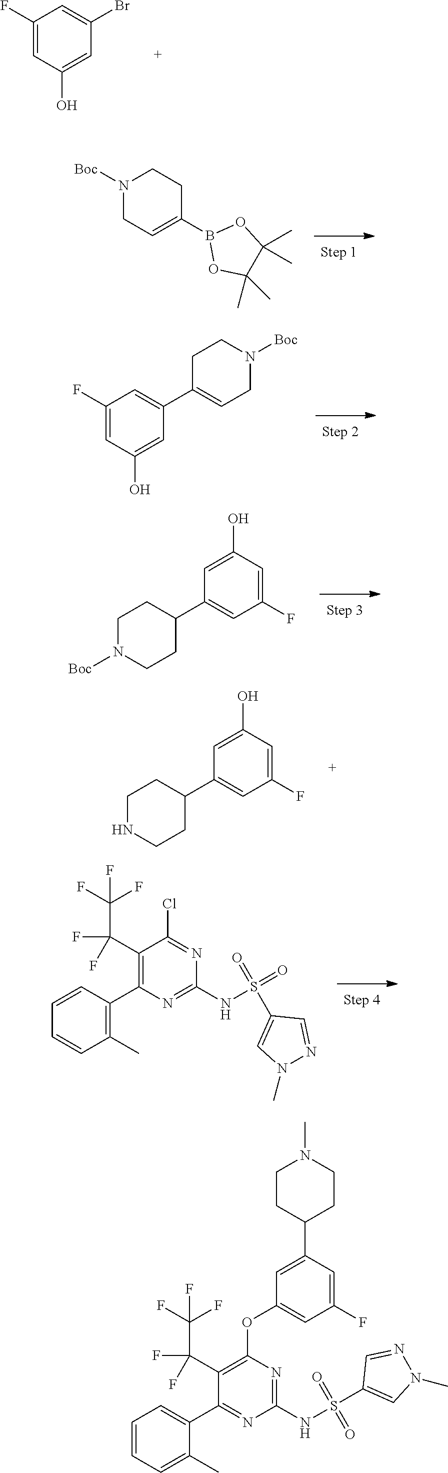

### Step 1: 5-Fluoro-3-(N-tert-butyloxycarbonyl-2,3,6-trihydropyridin-4-yl)phenol

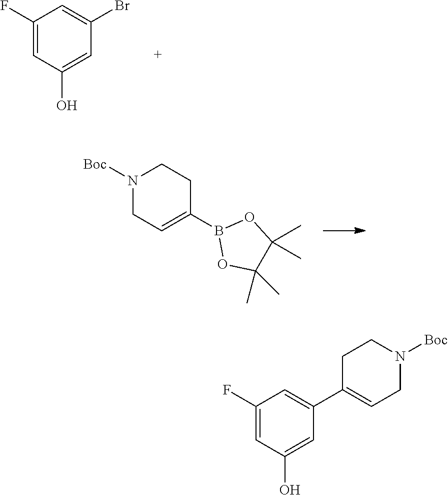 [SMILES: Oc1cc(F)cc(Br)c1 ; CC(C)(C)OC(=O)N1CC=C(B2OC(C)(C)C(C)(C)O2)CC1 ; CC(C)(C)OC(=O)N1CC=C(c2cc(O)cc(F)c2)CC1]

To a solution of 5-fluoro-3-bromophenol (2.0 g, 10.5 mmol) in dioxane (10 mL) and 2 M aqueous potassium carbonate solution (10 mL) were added N-tert-butoxycarbonyl-2,3,6-trahydropyridine-4-boronic acid, pinacol ester (4.0 g, 12.9 mmol) and dichloro 1,1′-bis(diphenylphosphino)ferrocene palladium (II) dichloromethane (0.25 g, 0.31 mmol). The mixture was irradiated in microwave oven for 25 minutes at 140° C. Six other batches were run under the same conditions and all mixtures were combined for work-up. The reaction mixture was diluted with water (250 mL) and extracted with ethyl acetate (3×250 mL). The organic layer was washed with brine, dried over sodium sulfate and concentrated. The crude residue was purified by silica gel column chromatography using 0-40% hexanes—ethyl acetate to afford 5-fluoro-3-(N-tert-butyloxycarbonyl-2,3,6-trihydropyridin-4-yl)phenol (17.3 g, 94%) as an off-white solid. ESI-MS m/z: calc.293.14, found 294.3 (M+1). Retention time: 3.61 minutes.

### Step 2: 5-Fluoro-3-(N-tert-butoxycarbonyl-piperidin-4-yl)phenol

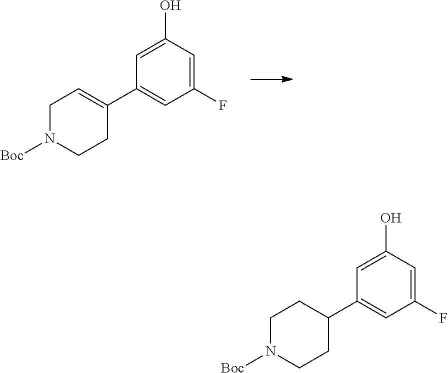 [SMILES: CC(C)(C)OC(=O)N1CC=C(c2cc(O)cc(F)c2)CC1 ; CC(C)(C)OC(=O)N1CCC(c2cc(O)cc(F)c2)CC1]

To a solution of 5-fluoro-3-(N-tert-butyloxycarbonyl-2,3,6-trahydropyridin-4-yl)phenol (17.3 g, 59.01 mmol) in methanol (100 mL) were added 10% palladium on carbon (2.0 g) and the mixture was stirred under hydrogen atmosphere at 60 psi for 2 hours. The reaction mixture was filtered through Celite pad and the filtrate was concentrated to give 5-fluoro-3-(N-tert-butoxycarbonyl-piperidin-4-yl)phenol (15.8 g, 90%) as an off-white solid. ESI-MS m/z: calc. 295.16, found 296.4 (M+1). Retention time: 3.57 minutes.

### Step 3: 5-Fluoro-3-(piperidin-4-yl)phenol

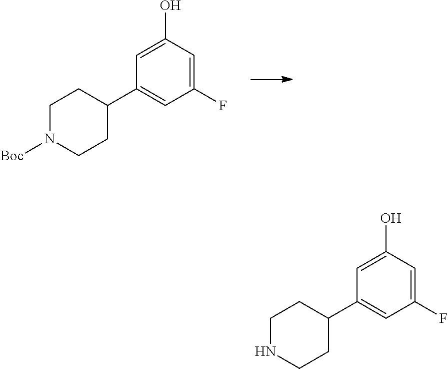 [SMILES: CC(C)(C)OC(=O)N1CCC(c2cc(O)cc(F)c2)CC1 ; Oc1cc(F)cc(C2CCNCC2)c1]

To a stirred solution of 5-fluoro-3-(N-tert-butoxycarbonyl-piperidin-4-yl)phenol (15.8 g, 53.52 mmol) in dichloromethane (50 mL) was added a 4 N hydrogen chloride solution in dioxane (60 mL). The mixture was stirred for 1 hour at room temperature and concentrated. Diethyl ether (100 mL) was added to the residue and the resulting precipitate was collected by filtration to afford 5-fluoro-3-(piperidin-4-yl)phenol (hydrochloride salt (14.6 g, >100%) as a white solid. The salt was dissolved in water (60 mL) and saturated aqueous sodium bicarbonate solution was added slowly to pH=8-10. The aqueous layer was extracted with ethyl acetate (2×200 mL). The organic layer was washed with brine, dried over sodium sulfate and concentrated to give 5-fluoro-3-(piperidin-4-yl)phenol (12.10 g, >100%) as a clear gel. ESI-MS m/z: calc. 195.11, found 196.2 (M+1). Retention time: 2.01 minutes.

### Step 4: 5-Fluoro-3-(N-methylpiperidin-4-yl)phenol

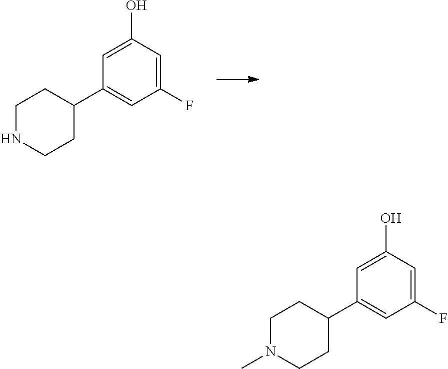

To a solution of 5-fluoro-3-(piperidin-4-yl)phenol (12.1 g, 53.52 mmol) in methanol (100 mL) and 37% aqueous formaldehyde solution (40 mL, 480 mmol) were added 10% palladium on carbon (1.7 g). The mixture was stirred under hydrogen atmosphere at 60 psi for 1 hour, filtered and concentrated. Saturated aqueous sodium bicarbonate solution (70 mL) and ethyl acetate (300 mL) were added. The organic layer was washed with brine, dried over sodium sulfate and concentrated to afford 5-fluoro-3-(N-methylpiperidin-4-yl)phenol (6.89 g, 61%) as an off-white solid. ESI-MS m/z: calc. 209.12, found 210.2 (M+1). Retention time: 2.56 minutes.

¹H NMR (250 MHz, CDCl₃) δ (ppm): 6.20-6.45 (m, 3H), 2.95-3.10 (m, 2H), 2.20-2.40 (m, 1H), 2.31 (s, 3H), 2.05-2.15 (m, 2H), 1.45-1.75 (m, 4H).

### Step 5: N-[4-[3-fluoro-5-(1-methyl-4-piperidyl)phenoxy]-6-(o-tolyl)-5-(1,1,2,2,2-pentafluoroethyl)pyrimidin-2-yl]-1-methyl-pyrazole-4-sulfonamide

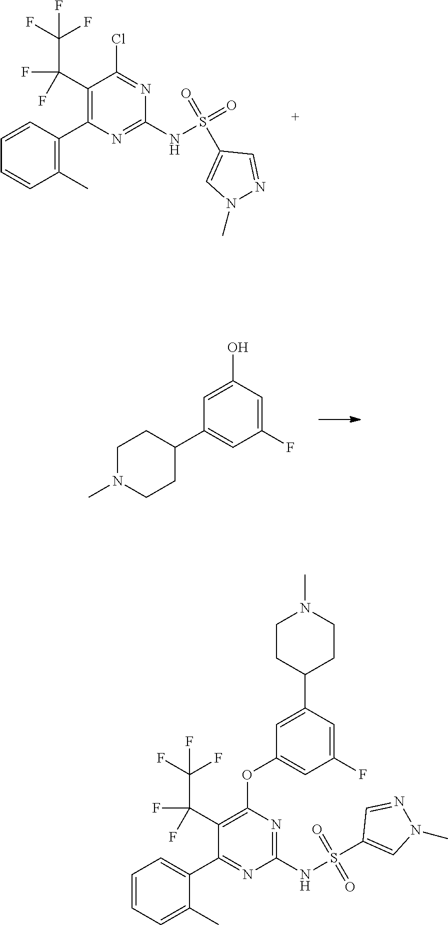 [SMILES: Cc1ccccc1-c1nc(NS(=O)(=O)c2cnn(C)c2)nc(Cl)c1C(F)(F)C(F)(F)F ; CN1CCC(c2cc(O)cc(F)c2)CC1 ; Cc1ccccc1-c1nc(NS(=O)(=O)c2cnn(C)c2)nc(Oc2cc(F)cc(C3CCN(C)CC3)c2)c1C(F)(F)C(F)(F)F]

To a 3-mL vial equipped with a magnetic stir bar, N-[4-chloro-6-(o-tolyl)-5-(1,1,2,2,2-pentafluoroethyl)pyrimidin-2-yl]-1-methyl-pyrazole-4-sulfonamide (16.30 mg, 0.03383 mmol), 3-fluoro-5-(1-methyl-4-piperidyl)phenol (24.04 mg, 0.1149 mmol), K₂CO₃ (21.50 mg, 0.1556 mmol) and NMP (500 μL) were added. This slurry was stirred at 120° C. for 13 hours. The reaction mixture was then cooled to room temperature, diluted with MeOH (400 μL), filtered and purified by reverse phase HPLC (1-70% acetonitrile in water using HCl as a modifier) to give the desired product N-[4-[3-fluoro-5-(1-methyl-4-piperidyl)phenoxy]-6-(o-tolyl)-5-(1,1,2,2,2-pentafluoroethyl)pyrimidin-2-yl]-1-methyl-pyrazole-4-sulfonamide (hydrochloride salt) (7.9 mg, 34%). ESI-MS m/z calc. 654.18475, found 655.4 (M+1)⁺; Retention time: 1.56 minutes; LC method A.

### Example 390: Preparation of Compound 1416

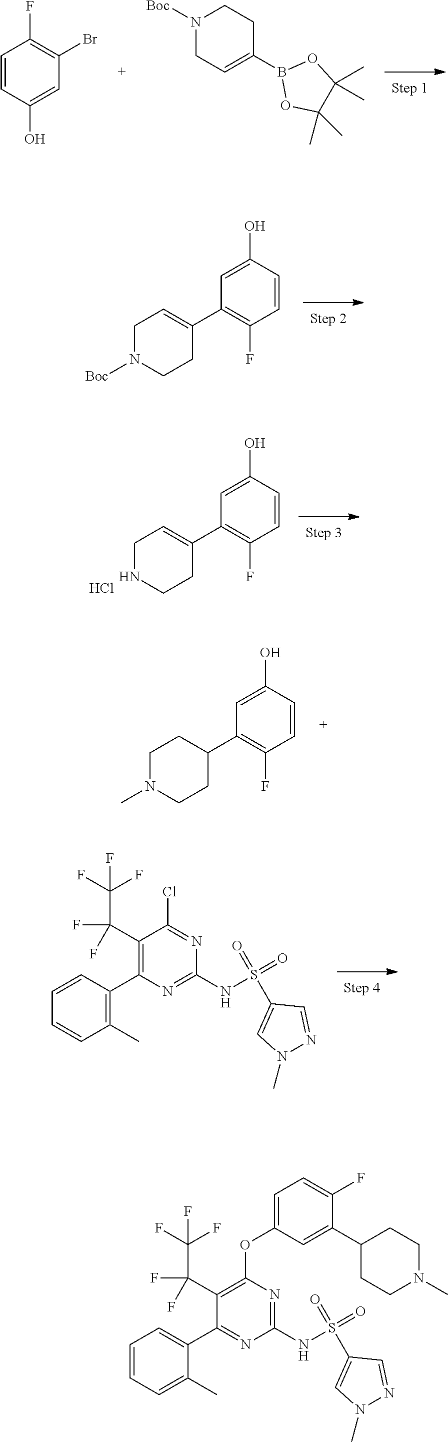

### Step 1: 4-Fluoro-3-(N-tert-butyloxycarbonyl-2,3,6-trihydropyridin-4-yl)phenol

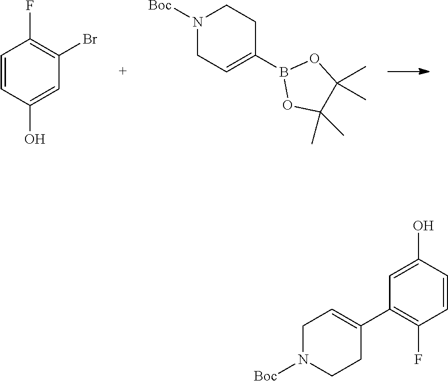 [SMILES: Oc1ccc(F)c(Br)c1 ; CC(C)(C)OC(=O)N1CC=C(B2OC(C)(C)C(C)(C)O2)CC1 ; CC(C)(C)OC(=O)N1CC=C(c2cc(O)ccc2F)CC1]

To a solution of 4-fluoro-3-bromophenol (2.37 g, 12.4 mmol) in dioxane (12 mL) and 2 M aqueous potassium carbonate solution (10 mL) were added N-tert-butoxycarbonyl-2,3,6-trahydropyridine-4-boronic acid, pinacolate (6 g, 19.4 mmol) and dichloro 1,1′-bis(diphenylphosphino)ferrocene palladium (II) dichloromethane adduct (0.4 g, 0.49 mmol). The mixture was irradiated in microwave for 25 minutes at 140° C. The reaction was run in 4 batches the reaction mixtures were combined for workup, diluted with water (200 mL) and extracted with ethyl acetate (3×200 mL). The organic layer was washed with brine, dried over sodium sulfate and concentrated. The crude residue was purified by silica gel column chromatography using 0-40% hexanes—ethyl acetate to afford 4-fluoro-3-(N-tert-butyloxycarbonyl-2,3,6-trihydropyridin-4-yl)phenol (14.1 g, 97%) as an off-white solid. ESI-MS m/z: calc.293.14, found 294.5 (M+1). Retention time: 3.26 minutes.

### Step 2: 4-Fluoro-3-(2,3,6-trihydropyridin-4-yl)phenol

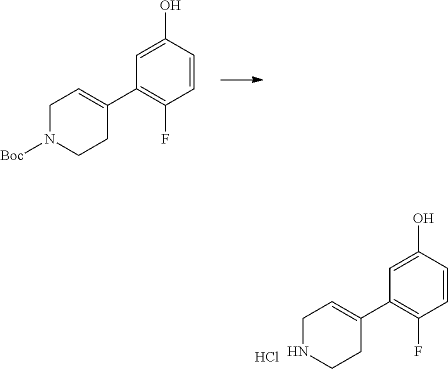 [SMILES: CC(C)(C)OC(=O)N1CC=C(c2cc(O)ccc2F)CC1 ; Cl ; Oc1ccc(F)c(C2=CCNCC2)c1]

To a stirred solution of 4-fluoro-3-(N-tert-butyloxycarbonyl-2,3,6-trihydropyridin-4-yl)phenol (14.1 g, 48.10 mmol) in dichloromethane (50 mL) was added 4 N hydrogen chloride solution in dioxane (60 mL). The mixture was stirred for 1 hour at room temperature and concentrated. Diethyl ether (100 mL) was added to the residue and formed precipitate was collected by filtration to afford 4-fluoro-3-(2,3,6-trihydropyridin-4-yl)phenol hydrochloric acid salt (7.6 g, 69%) as an off-white solid. ESI-MS m/z: calc. 193.09, found 194.1 (M+1). Retention time: 1.82 minutes.

### Step 3: 4-Fluoro-3-(N-methylpiperidin-4-yl)phenol

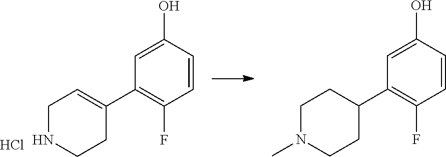

To a solution of 4-fluoro-3-(2,3,6-trihydropyridin-4-yl)phenol hydrochloride salt (7.6 g, 53.52 mmol) in methanol (100 mL) were added triethylamine (10 mL), a 37% aqueous formaldehyde solution (40 mL, 480 mmol) and 10% palladium on carbon (1.5 g). The mixture was stirred under hydrogen atmosphere at 50 psi for 1 hours. The reaction was filtered and concentrated, then saturated sodium bicarbonate (50 mL) and ethyl acetate (200 mL) were added and the two phases were separated. The organic layer was washed with brine, dried over sodium sulfate and concentrated. The crude residue was dissolved in methanol (50 mL). 10% palladium on carbon (2.0 g) was added. The mixture was stirred under a hydrogen atmosphere at 60 psi for 16 hours. The reaction mixture was filtered through Celite pad and the filtrate was concentrated to give 4-fluoro-3-(N-methylpiperidin-4-yl)phenol (4.5 g, 45%) as an off-white solid. ESI-MS m/z: calc. 209.12, found 210.2 (M+1). Retention time: 2.59 minutes. ¹H NMR (250 MHz, CDCl₃) δ (ppm): 6.55-6.95 (m, 3H), 3.00-3.10 (m, 2H), 2.75-2.90 (m, 1H), 2.34 (s, 3H), 2.015-2.25 (m, 2H), 1.60-1.80 (m, 4H).

### Step 4: N-[4-[4-fluoro-3-(1-methyl-4-piperidyl)phenoxy]-6-(o-tolyl)-5-(1,1,2,2,2-pentafluoroethyl)pyrimidin-2-yl]-1-methyl-pyrazole-4-sulfonamide

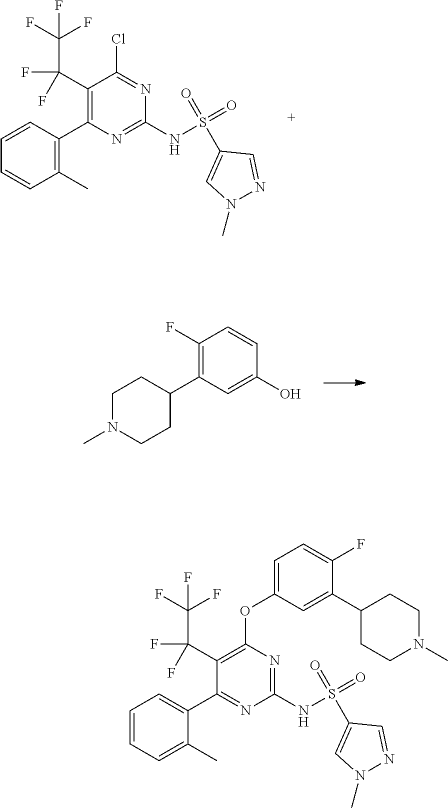 [SMILES: Cc1ccccc1-c1nc(NS(=O)(=O)c2cnn(C)c2)nc(Cl)c1C(F)(F)C(F)(F)F ; CN1CCC(c2cc(O)ccc2F)CC1 ; Cc1ccccc1-c1nc(NS(=O)(=O)c2cnn(C)c2)nc(Oc2ccc(F)c(C3CCN(C)CC3)c2)c1C(F)(F)C(F)(F)F]

To a 3-mL vial equipped with a magnetic stir bar, N-[4-chloro-6-(o-tolyl)-5-(1,1,2,2,2-pentafluoroethyl)pyrimidin-2-yl]-1-methyl-pyrazole-4-sulfonamide (16.30 mg, 0.03383 mmol), 4-fluoro-3-(1-methyl-4-piperidyl)phenol (24.04 mg, 0.1149 mmol), K₂CO₃ (21.50 mg, 0.1556 mmol) and NMP (500 μL) were added. This slurry was stirred at 120° C. for 13 hours. The reaction mixture was then cooled to room temperature, diluted with MeOH (400 L), filtered and purified by reverse phase HPLC (1-70% acetonitrile in water using HCl as a modifier) to give the desired product N-[4-[4-fluoro-3-(1-methyl-4-piperidyl)phenoxy]-6-(o-tolyl)-5-(1,1,2,2,2-pentafluoroethyl)pyrimidin-2-yl]-1-methyl-pyrazole-4-sulfonamide (hydrochloride salt) (9.2 mg, 39%). ESI-MS m/z calc. 654.18475, found 655.4 (M+1)⁺; Retention time: 1.52 minutes; LC method A. ¹H NMR (400 MHz, dimethylsulfoxide-d₆) δ 12.79 -12.02 (br s, 1H, D20 exchangeable), 10.62-9.70 (br s, 1H, D20 exchangeable), 7.74-7.64 (m, 1H), 7.46-7.40 (m, 1H), 7.39-7.34 (m, 1H), 7.33-7.29 (m, 1H), 7.29-7.18 (m, 3H), 7.17-7.11 (m, 1H), 6.94-6.83 (m, 1H), 3.76 (s, 3H), 3.56-3.47 (m, 2H), 3.18-3.08 (m, 2H), 2.90-2.73 (m, 3H), 2.12-2.01 (m, 3H), 2.09 (s, 3H), [Note: 2H are missing from the overall hydrogen count of 29 from the product (C₂₉H₂₈F6N 6O3S+HCl)].

### Example 391: Preparation of Compound 1417

### Step 1. 4-(3-Fluoro-4-hydroxy-phenyl)-3, 6-dihydro-2H-pyridine-1-carboxylic acid tert-butyl ester

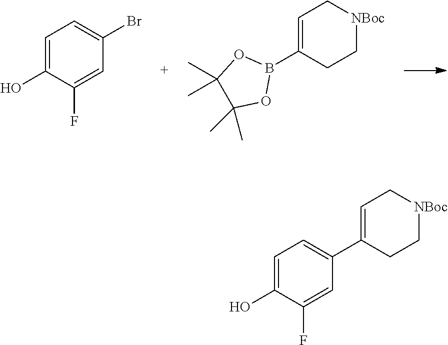 [SMILES: Oc1ccc(Br)cc1F ; CC(C)(C)OC(=O)N1CC=C(B2OC(C)(C)C(C)(C)O2)CC1 ; CC(C)(C)OC(=O)N1CC=C(c2ccc(O)c(F)c2)CC1]

To a solution of 4-bromo-2-fluorophenol (4.8 g, 25 mmol) and N-Boc-piperidine-4-boronic acid pinacol ester (11.7 g, 37.5 mmol) in nitrogen purged dioxane/water(1:1 ratio, 100mL) was added Na₂CO₃ (8 g, 75 mmol) and Pd(PPh₃)₄ (578 mg, 0.5 mmol). The mixture was stirred at 120° C. under microwave for 1 hours. The pale-yellow mixture was then filtered. The filtrate was extracted with ethyl acetate (5×150 mL). The combined organic layer was washed with 5% sodium bicarbonate (300 mL), brine (300 mL), dried over sodium sulfate, filtered and concentrated. The residue was purified by silica gel chromatography eluting with a gradient of 0% to 100% of EtOAc in hexanes to afford 4-(3-fluoro-4-hydroxy-phenyl)-3,6-dihydro-2H-pyridine-1-carboxylic acid tert-butyl ester (5.9 g, 20 mmol, 80% yield) as a pale-yellow powder. ESI-MS m/z calc. 293.33, found 294.2 (M+1)⁺; Retention time: 2.91 minutes (LC method B).

### Step 2. 4-(3-Fluoro-4-hydroxy-phenyl)-piperidine-1-carboxylic acid tert-butyl ester

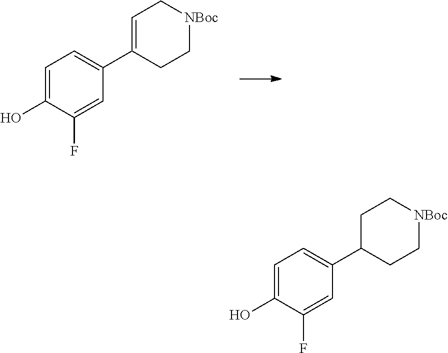 [SMILES: CC(C)(C)OC(=O)N1CC=C(c2ccc(O)c(F)c2)CC1 ; CC(C)(C)OC(=O)N1CCC(c2ccc(O)c(F)c2)CC1]

In a 2 L hydrogenation vessel, 4-(3-fluoro-4-hydroxy-phenyl)-3,6-dihydro-2H-pyridine-1-carboxylic acid tert-butyl ester (5.9 g, 20 mmole) in methanol (500 mL) was vacuumed and refilled with nitrogen. Palladium on carbon (1.3 g, 10% w/w, 50% wet) was then added. The vessel was placed on a Parr shaker, purged twice with hydrogen and it was hydrogenated at 60 psi hydrogen for 2 hours. After purging with nitrogen, the mixture was treated with 3 N HCl in methanol until the pH was ˜ 2. The mixture was filtered through a pad of Celite and washed with methanol (˜300 mL). The filtrate was concentrated and used for next step without further purification. ESI-MS m/z calc. 295.35, found 296.4 (M+1)⁺; Retention time: 2.92 minutes (LC method b).

### Step 3. 2-Fluoro-4-piperidin-4-yl-phenol

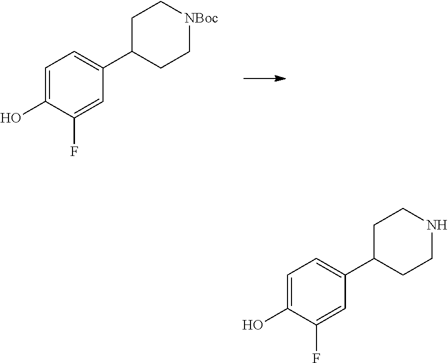 [SMILES: CC(C)(C)OC(=O)N1CCC(c2ccc(O)c(F)c2)CC1 ; Oc1ccc(C2CCNCC2)cc1F]

4-(3-Fluoro-4-hydroxy-phenyl)-piperidine-1-carboxylic acid tert-butyl ester from step 2 was dissolved and stirred in 50% TFA/DCM (150 mL) for 20 min at room temperature. The reactants were removed under vacuum, and the residue was washed with cold ether and dried under high vacuum to afford 2-fluoro-4-piperidin-4-yl-phenol (3.7 g, 95% yield) as a pale yellow solid. ESI-MS m/z calc. 195.23, found 195.8 (M+1)⁺; Retention time: 1.42 minutes (LC method B).

### Step 4. 2-Fluoro-4-(1-methyl-piperidin-4-yl)-phenol

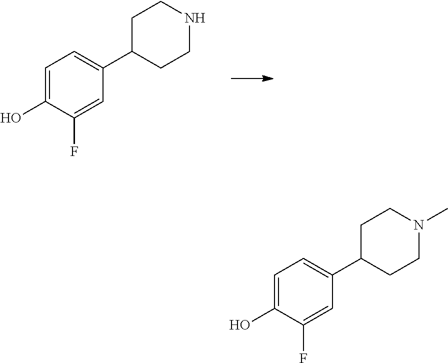

To a solution of 2-fluoro-4-piperidin-4-yl-phenol (3.7 g, 19 mmol) in THE (50 mL) was added formaldehyde (37% in water, 9.2 mL). The mixture was cooled and stirred at 0° C. for 30 minutes. Sodium triacetoxyborohydride (5.6 g, 27 mmole) was added portion-wise and the reaction was allowed to warm to room temperature for 20 hours. The THF was evaporated and the resulting solid was dissolved in DCM (200 mL). The pH was adjusted to pH 8-9 with saturated aqueous sodium bicarbonate and the mixture was stirred for 10 minutes. The layers were separated and the aqueous layer was extracted with DCM (6×300 mL). The combined organic layers were dried over sodium sulfate, filtered, and concentrated under vacuum, to afford 2-fluoro-4-(1-methyl-piperidin-4-yl)-phenol (3.55 g, 96%) ESI-MS m/z calc. 209.3, found 210.0 (M+1)⁺; Retention time: 0.79 minutes (LC method B).

### Step 5: N-[4-[2-fluoro-4-(1-methyl-4-piperidyl)phenoxy]-6-(o-tolyl)-5-(1,1,2,2,2-pentafluoroethyl)pyrimidin-2-yl]-1-methyl-pyrazole-4-sulfonamide

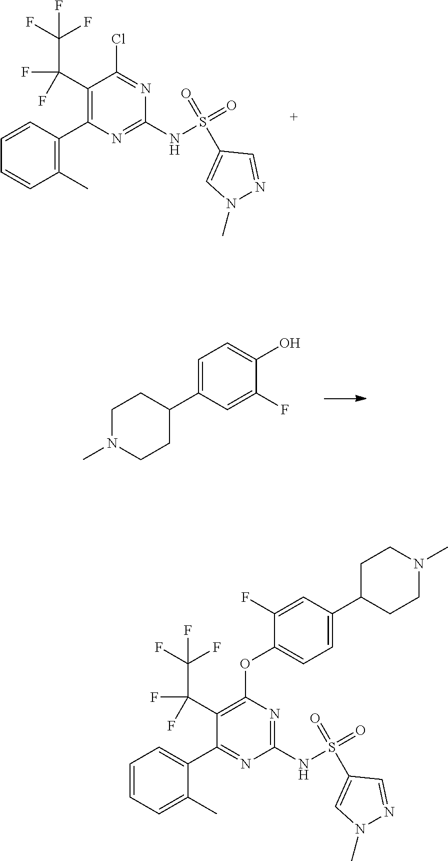 [SMILES: Cc1ccccc1-c1nc(NS(=O)(=O)c2cnn(C)c2)nc(Cl)c1C(F)(F)C(F)(F)F ; CN1CCC(c2ccc(O)c(F)c2)CC1 ; Cc1ccccc1-c1nc(NS(=O)(=O)c2cnn(C)c2)nc(Oc2ccc(C3CCN(C)CC3)cc2F)c1C(F)(F)C(F)(F)F]

To a 3-mL vial equipped with a magnetic stir bar, N-[4-chloro-6-(o-tolyl)-5-(1,1,2,2,2-pentafluoroethyl)pyrimidin-2-yl]-1-methyl-pyrazole-4-sulfonamide (16.30 mg, 0.03383 mmol), 2-fluoro-4-(1-methyl-4-piperidyl)phenol (24.04 mg, 0.1149 mmol), K₂CO₃ (21.50 mg, 0.1556 mmol) and NMP (500 μL) were added. This slurry was stirred at 120° C. for 13 hours. The reaction mixture was then cooled to room temperature, diluted with MeOH (400 L), filtered and purified by reverse phase HPLC (1-70% acetonitrile in water using HCl as a modifier) to give the desired product N-[4-[2-fluoro-4-(1-methyl-4-piperidyl)phenoxy]-6-(o-tolyl)-5-(1,1,2,2,2-pentafluoroethyl)pyrimidin-2-yl]-1-methyl-pyrazole-4-sulfonamide (hydrochloride salt) (7.7 mg, 33%). ESI-MS m/z calc. 654.18475, found 655.4 (M+1)⁺; Retention time: 1.54 minutes; LC method A.

### Example 392: Preparation of Compound 1418

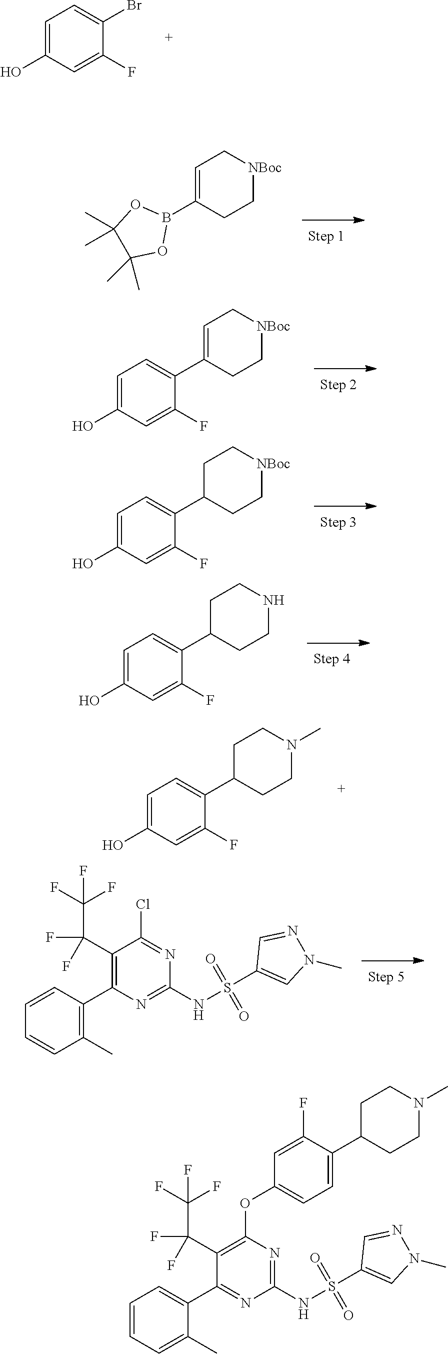

### Step 1. 4-((2-Fluoro-4-hydoxy-phenyl)-3, 6-dihydro-2H-pyridine-1-carboxylic acid tert-butyl ester

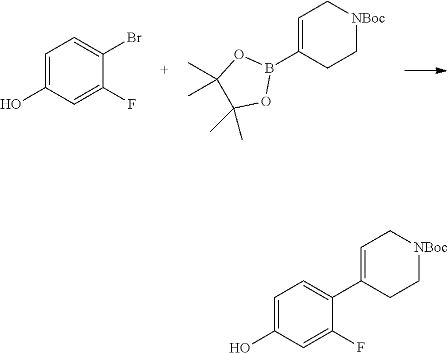 [SMILES: Oc1ccc(Br)c(F)c1 ; CC(C)(C)OC(=O)N1CC=C(B2OC(C)(C)C(C)(C)O2)CC1 ; CC(C)(C)OC(=O)N1CC=C(c2ccc(O)cc2F)CC1]

To a solution of 4-bromo-3-fluorophenol (3.9 g, 20 mmol) and N-Boc-piperidine-4-boronic acid pinacol ester (9.4 g, 30 mmol) in nitrogen purged dioxane/water(1:1 ratio, 80 mL) was added Na₂CO₃ (6.4 g, 60 mmol) and Pd(PPh₃)₄ (462 mg, 0. 4 mmol). The reaction was stirred at 120° C. under microwave for 1 hour. The pale-yellow solution was then filtered. The filtrate was extracted with ethyl acetate (3×150 mL). The combined organic layer was washed with 5% sodium bicarbonate (300 mL), brine (300 mL), dried over sodium sulfate, filtered and concentrated. The residue was purified by silica gel chromatography (120 g column), eluting with 0% to 100% EtOAc in hexanes to afford 4-(2-fluoro-4-hydroxy-phenyl)-3,6-dihydro-2H-pyridine-1-carboxylic acid tert-butyl ester (4.7 g, 16 mmol, 80% yield) as a pale yellow powder. ESI-MS m/z calc. 293.33, found 294.2 (M+1)⁺; Retention time: 2.92 minutes (LC method B).

### Step 2. 4-(2-Fluoro-4-hydroxy-phenyl)-piperidine-1-carboxylic acid tert-butyl ester

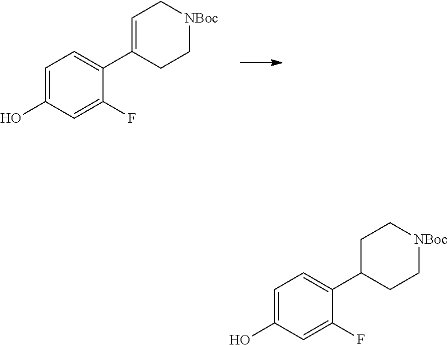 [SMILES: CC(C)(C)OC(=O)N1CC=C(c2ccc(O)cc2F)CC1 ; CC(C)(C)OC(=O)N1CCC(c2ccc(O)cc2F)CC1]

In a 2 L hydrogenation vessel, 4-(2-fluoro-4-hydroxy-phenyl)-3,6-dihydro-2H-phridine-1-carboxylic acid tert-butyl ester (4.7 g, 16 mmole) in methanol (450 mL) was vacuumed and refilled with nitrogen. Palladium on carbon (1.0 g, 10% w/w, 50% wet), was then added. The vessel was placed on a Parr shaker, purged twice with hydrogen and hydrogenated at 60 psi of hydrogen gas for 2 hours. After purging with nitrogen, the mixture was treated with 3 N HCl in methanol until the pH was ˜ 2. The mixture was then filtered through a Celite pad and washed with methanol (˜300 mL). The filtrate was concentrated and used for next step without further purification. ESI-MS m/z calc. 295.35, found 296 (M+1)⁺; Retention time: 2.91 minutes (LC method B).

### Step 3. 3-Fluoro-4-piperidin-4-yl-phenol

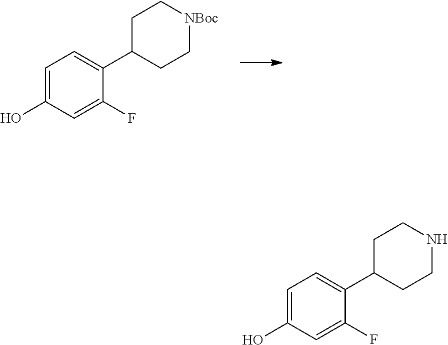 [SMILES: CC(C)(C)OC(=O)N1CCC(c2ccc(O)cc2F)CC1 ; Oc1ccc(C2CCNCC2)c(F)c1]

4-(2-Fluoro-4-hydroxy-phenyl)-piperidine-1-carboxylic acid tert-butyl ester was treated with 50% TFA in DCM(150 ml) for 20 min at room temperature. TFA/DCM were removed under vacuum and then the residue was washed with cold ether. The resulting solid was dried under high vacuum to afford 3-fluoro-4-piperidin-4-yl-phenol (3.0 g, 95% yield) as a pale yellow solid. ESI-MS m/z calc. 195.23, found 195.5 (M+1)⁺; Retention time: 1.29 minutes (LC method B).

### Step 4. 3-Fluoro-4-(1-methyl-piperidin-4-yl)-phenol

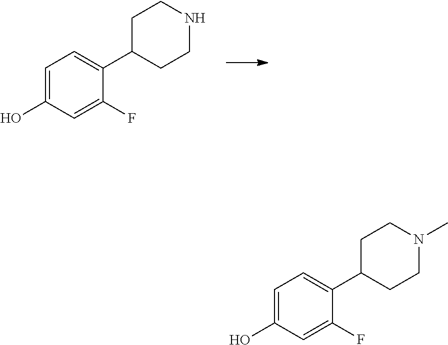

In a 250 ml hydrogenation vessel, 3-fluoro-4-piperidine-4-yl-phenol (3.0 g, 15.4 mmol) and aqueous formaldehyde (37% in water, 7.5 ml, excess) in methanol (40 mL) was vacuumed and refilled with nitrogen. Palladium on carbon (0.9 g, 10% w/w, 50% wet), was then added. The vessel was purged twice with hydrogen and then was hydrogenated at 1 atm hydrogen for 4 hours. After purging with nitrogen, the mixture was treated with 3 N HCl in methanol until the pH was ˜ 2. The mixture was then filtered through a pad of Celite and washed with methanol (˜200 mL). The filtrate was dried under vacuum to afford 2-fluoro-4-(1 -methyl-piperidin-4-yl)-phenol (3.1 g, 94.4% purity, 98% yield). ESI-MS m/z calc. 209.3, found 210.0 (M+1)⁺; Retention time: 1.02 minutes (LC method B).

### Step 5: N-[4-[3-fluoro-4-(1-methyl-4-piperidyl)phenoxy]-6-(o-tolyl)-5-(1,1,2,2,2-pentafluoroethyl)pyrimidin-2-yl]-1-methyl-pyrazole-4-sulfonamide

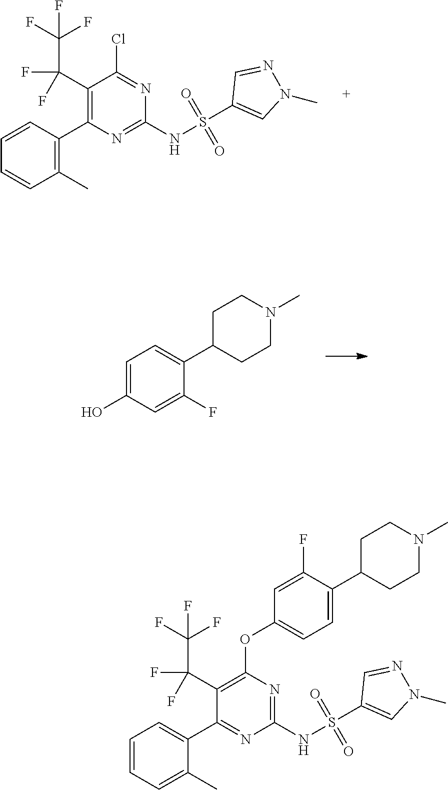 [SMILES: Cc1ccccc1-c1nc(NS(=O)(=O)c2cnn(C)c2)nc(Cl)c1C(F)(F)C(F)(F)F ; CN1CCC(c2ccc(O)cc2F)CC1 ; Cc1ccccc1-c1nc(NS(=O)(=O)c2cnn(C)c2)nc(Oc2ccc(C3CCN(C)CC3)c(F)c2)c1C(F)(F)C(F)(F)F]

To a 3-mL vial equipped with a magnetic stir bar, N-[4-chloro-6-(o-tolyl)-5-(1,1,2,2,2-pentafluoroethyl)pyrimidin-2-yl]-1-methyl-pyrazole-4-sulfonamide (16.30 mg, 0.03383 mmol), 3-fluoro-4-(1-methyl-4-piperidyl)phenol (24.04 mg, 0.1149 mmol), K₂CO₃ (21.50 mg, 0.1556 mmol)) and NMP (500 μL) were added. This slurry was stirred at 120° C. for 13 hours. The reaction mixture was then cooled to room temperature, diluted with MeOH (400 L), filtered and purified by reverse phase HPLC (1-70% acetonitrile in water using HCl as a modifier) to give the desired product N-[4-[3-fluoro-4-(1-methyl-4-piperidyl)phenoxy]-6-(o-tolyl)-5-(1,1,2,2,2-pentafluoroethyl)pyrimidin-2-yl]-1-methyl-pyrazole-4-sulfonamide (hydrochloride salt) (8.3 mg, 36%). ESI-MS m/z calc. 654.18475, found 655.4 (M+1)⁺; Retention time: 1.55 minutes; LC method A.

### Example 393: Preparation of Compound 1419

### Step 1: 2-Fluoro-3-(4-methylpiperazin-1-yl)phenol

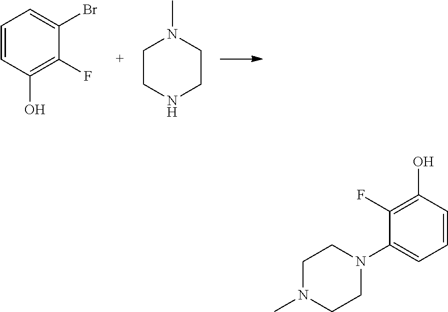

A 1 M solution of lithium bis(trimethylsilyl)amide in tetrahydrofuran (200 mL, 200 mmol) was added to a mixture of 3-bromo-2-fluorophenol (13.1 g, 68.6 mmol), 1-methylpiperazine (4.7 mL, 68.4 mmol), 2,8,9-triisobutyl-2,5,8,9-tetraza-1-phospha bicyclo{3.3.3}undecane (0.49 g, 1.4 mmol) and palladium (II) acetate (0.5 g, 1.8 mmol) in toluene (60 mL) and the reaction mixture was subjected to three cycles of evacuation-backfilling with argon in a Schlenk flask. The reaction mixture was heated at 50° C. for 13 hours. The mixture was filtered through a Celite pad and concentrated to dryness. The residue was purified by silica gel column chromatography using 0-10% dichloromethane-methanol to give 2-fluoro-3-(4-methylpiperazin-1-yl)phenol (1.0 g, 7%) as an off white solid. ESI-MS m/z calc. 210.12, found 211.2 (M+1). Retention time: 1.11 minutes ¹H NMR (250 MHz, DMSO) δ (ppm): 9.76 (s, 1H), 6.86 (m, 1H), 6.66 (m, 1H), 6.47 (m, 1H), 3.60-3.00 (br, 8H), 2.85 (s, 3H).

### Step 2: N-[4-[2-fluoro-3-(4-methylpiperazin-1-yl)phenoxy]-6-(o-tolyl)-5-(1,1,2,2,2-pentafluoroethyl)pyrimidin-2-yl]-1-methyl-pyrazole-4-sulfonamide

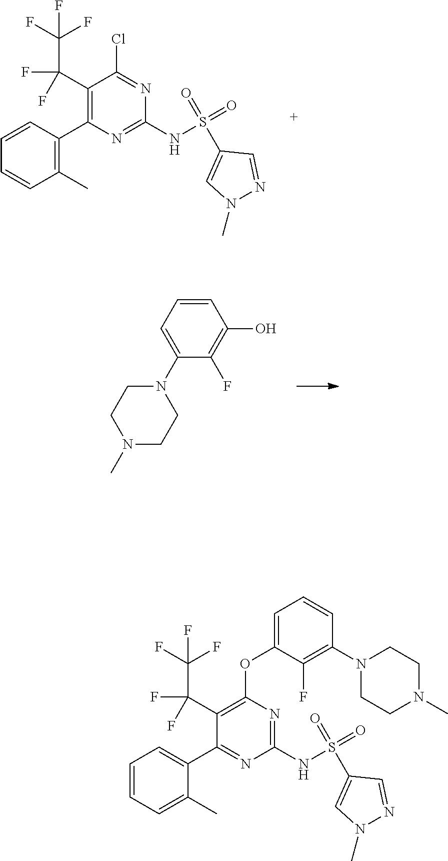 [SMILES: Cc1ccccc1-c1nc(NS(=O)(=O)c2cnn(C)c2)nc(Cl)c1C(F)(F)C(F)(F)F ; CN1CCN(c2cccc(O)c2F)CC1 ; Cc1ccccc1-c1nc(NS(=O)(=O)c2cnn(C)c2)nc(Oc2cccc(N3CCN(C)CC3)c2F)c1C(F)(F)C(F)(F)F]

To a 3-mL vial equipped with a magnetic stir bar, N-[4-chloro-6-(o-tolyl)-5-(1,1,2,2,2-pentafluoroethyl)pyrimidin-2-yl]-1-methyl-pyrazole-4-sulfonamide (16.30 mg, 0.03383 mmol), 2-fluoro-3-(4-methylpiperazin-1-yl)phenol (24.16 mg, 0.1149 mmol), K₂CO₃ (21.50 mg, 0.1556 mmol) and NMP (500 μL) were added. This slurry was stirred at 120° C. for 13 hours. The reaction mixture was then cooled to room temperature, diluted with MeOH (400 L), filtered and purified by reverse phase HPLC (1-70% acetonitrile in water using HCl as a modifier) to give the desired product N-[4-[2-fluoro-3-(4-methylpiperazin-1-yl)phenoxy]-6-(o-tolyl)-5-(1,1,2,2,2-pentafluoroethyl)pyrimidin-2-yl]-1-methyl-pyrazole-4-sulfonamide (hydrochloride salt) (8.7 mg, 37%). ESI-MS m/z calc. 655.18005, found 656.4 (M+1)⁺; Retention time: 1.52 minutes; LC method A. ¹H NMR (400 MHz, dimethylsulfoxide-d₆) δ 12.97-11.73 (br s, 1H, D20 exchangeable), 10.92-10.15 (br s, 1H, D20 exchangeable), 7.50 (s, 1H), 7.40-7.30 (m, 3H), 7.26 (td, J=7.4, 1.4 Hz, 1H), 7.22-7.15 (m, 2H), 7.15-7.09 (m, 1H), 7.03 (s, 1H), 3.78 (s, 3H), 3.67-3.57 (m, 2H), 3.56-3.46 (m, 2H), 3.28-3.12 (m, 4H), 2.83 (s, 3H), 2.07 (s, 3H)

### Example 394: Preparation of Compound 1420

### Step 1: 2-Fluoro-5-(4-methylpiperazin-1-yl)phenol

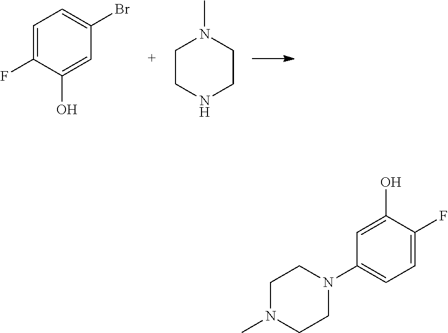

A 1 M solution of lithium bis(trimethylsilyl)amide in tetrahydrofuran (200 mL, 200 mmol) was added to a mixture of 3-bromo-6-fluorophenol (16.2 g, 84.8 mmol), 1-methylpiperazine (5.8 mL, 84.8 mmol), 2,8,9-triisobutyl-2,5,8,9-tetraza-1-phospha bicyclo{3.3.3}undecane (0.60 g, 1.7 mmol) and palladium (II) acetate (0.5 g, 2.1 mmol) in toluene (60 mL) and the reaction mixture was subjected to three cycles of evacuation-backfilling with argon in a Schlenk flask. The reaction mixture was heated at 80° C. for 13 hours. The mixture was filtered through Celite and concentrated to dryness. The residue was purified by silica gel column chromatography using 0-10% dichloromethane-methanol to give 2-fluoro-5-(4-methylpiperazin-1-yl)phenol (6.2 g, 35%) as an off-white solid. ESI-MS m/z calc. 210.12, found 211.2 (M+1). Retention time: 1.18 minutes. ¹H NMR (250 MHz, DMSO) δ (ppm): 9.71 (s, 1H), 7.10 (m, 1H), 6.56 (m, 1H), 6.40 (m, 1H), 3.70-2.95 (br, 8H), 2.85 (s, 3H).

### Step 2: N-[4-[2-fluoro-5-(4-methylpiperazin-1-yl)phenoxy]-6-(o-tolyl)-5-(1,1,2,2,2-pentafluoroethyl)pyrimidin-2-yl]-1-methyl-pyrazole-4-sulfonamide

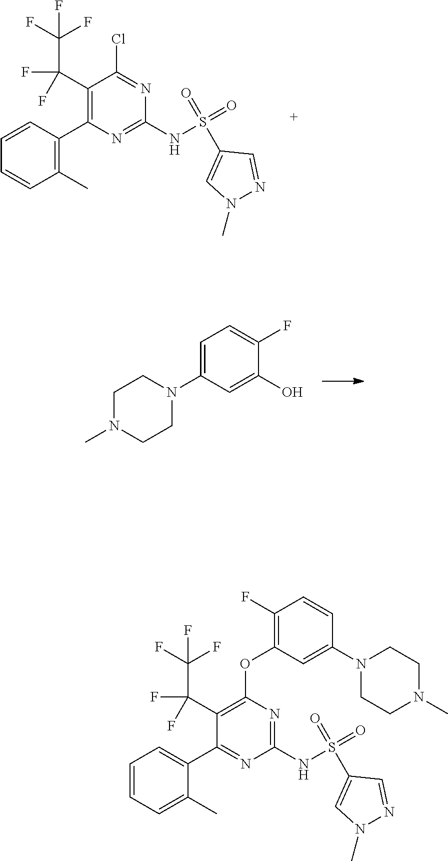 [SMILES: Cc1ccccc1-c1nc(NS(=O)(=O)c2cnn(C)c2)nc(Cl)c1C(F)(F)C(F)(F)F ; CN1CCN(c2ccc(F)c(O)c2)CC1 ; Cc1ccccc1-c1nc(NS(=O)(=O)c2cnn(C)c2)nc(Oc2cc(N3CCN(C)CC3)ccc2F)c1C(F)(F)C(F)(F)F]

To a 3-mL vial equipped with a magnetic stir bar, N-[4-chloro-6-(o-tolyl)-5-(1,1,2,2,2-pentafluoroethyl)pyrimidin-2-yl]-1-methyl-pyrazole-4-sulfonamide (16.30 mg, 0.03383 mmol), 2-fluoro-5-(4-methylpiperazin-1-yl)phenol (24.16 mg, 0.1149 mmol), K₂CO₃ (21.50 mg, 0.1556 mmol) and NMP (500 μL) were added. This slurry was stirred at 120° C. for 13 hours. The reaction mixture was then cooled to room temperature, diluted with MeOH (400 L), filtered and purified by reverse phase HPLC (1-70% acetonitrile in water using HCl as a modifier) to give the desired product N-[4-[2-fluoro-5-(4-methylpiperazin-1-yl)phenoxy]-6-(o-tolyl)-5-(1,1,2,2,2-pentafluoroethyl)pyrimidin-2-yl]-1-methyl-pyrazole-4-sulfonamide (hydrochloride salt) (5.9 mg, 25%). ESI-MS m/z calc. 655.18005, found 656.4 (M+1)⁺; Retention time: 1.51 minutes; LC method A.

### Example 395: Preparation of Compound 1421

Step 1: 4-Fluoro-3-(4-methylpiperazin-1-yl)phenol

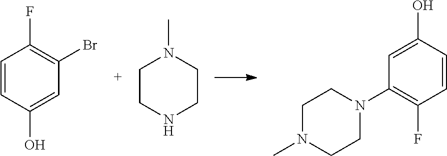

A 1 M solution of lithium bis(trimethylsilyl)amide in tetrahydrofuran (170 mL, 170 mmol) was added to a mixture of 3-bromo-4-fluorophenol (16.2 g, 84.8 mmol), 1-methylpiperazine (5.8 mL, 84.8 mmol), 2,8,9-triisobutyl-2,5,8,9-tetraza-1-phospha bicyclo{3.3.3}undecane (0.60 g, 1.7 mmol) and palladium (II) acetate (0.5 g, 2.1 mmol) in toluene (60 mL) and the reaction mixture was subjected to three cycles of evacuation-backfilling with argon in a Schlenk flask. The reaction mixture was heated at 80° C. for 13 hours. The mixture was filtered through Celite and concentrated to dryness. The residue was purified by silica gel column chromatography using 0-10% dichloromethane-methanol to give 4-fluoro-3-(4-methylpiperazin-1-yl)phenol (4.0 g, 22%) as an off-white solid. ESI-MS m/z calc. 210.12, found 211.2 (M+1). Retention time: 1.25 minutes. ¹H NMR (250 MHz, DMSO) δ (ppm): 9.37 (s, 1H), 6.96 (m, 1H), 6.41 (m, 2H), 3.60-2.95 (br, 8H), 2.86 (s, 3H).

### Step 2: N-[4-[4-fluoro-3-(4-methylpiperazin-1-yl)phenoxy]-6-(o-tolyl)-5-(1,1,2,2,2-pentafluoroethyl)pyrimidin-2-yl]-1-methyl-pyrazole-4-sulfonamide

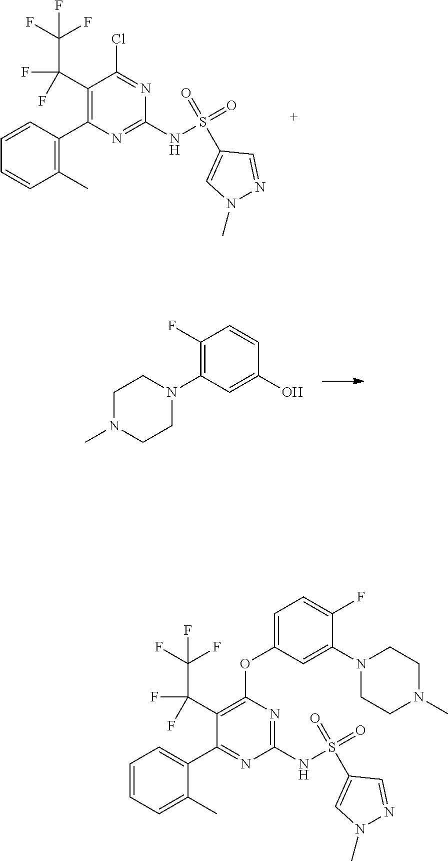 [SMILES: Cc1ccccc1-c1nc(NS(=O)(=O)c2cnn(C)c2)nc(Cl)c1C(F)(F)C(F)(F)F ; CN1CCN(c2cc(O)ccc2F)CC1 ; Cc1ccccc1-c1nc(NS(=O)(=O)c2cnn(C)c2)nc(Oc2ccc(F)c(N3CCN(C)CC3)c2)c1C(F)(F)C(F)(F)F]

To a 3-mL vial equipped with a magnetic stir bar, N-[4-chloro-6-(o-tolyl)-5-(1,1,2,2,2-pentafluoroethyl)pyrimidin-2-yl]-1-methyl-pyrazole-4-sulfonamide (16.30 mg, 0.03383 mmol), 4-fluoro-3-(4-methylpiperazin-1-yl)phenol (24.1 mg, 0.115 mmol), K₂CO₃ (21.5 mg, 0.156 mmol) and NMP (500 μL) were added. This slurry was stirred at 120° C. for 13 hours. The reaction mixture was then cooled to room temperature, diluted with MeOH (400 μL), filtered and purified by reverse phase HPLC (1-70% acetonitrile in water using HCl as a modifier) to give the desired product N-[4-[4-fluoro-3-(4-methylpiperazin-1-yl)phenoxy]-6-(o-tolyl)-5-(1,1,2,2,2-pentafluoroethyl)pyrimidin-2-yl]-1-methyl-pyrazole-4-sulfonamide (hydrochloride salt) (5.2 mg, 22%). ESI-MS m/z calc. 655.18005, found 656.4 (M+1)⁺; Retention time: 1.52 minutes; LC method A.

### Example 396: Preparation of Compound 1422

### Step 1: 3-Fluoro-5-(4-methylpiperazin-1-yl)phenol

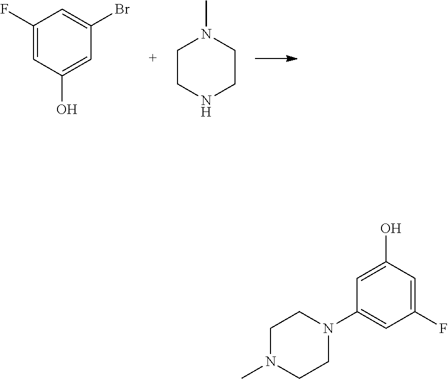

A 1 M solution of lithium bis(trimethylsilyl)amide in tetrahydrofuran (186 mL, 186 mmol) was added to a mixture of 3-bromo-5-fluorophenol (15 g, 78.5 mmol), 1-methylpiperazine (5.4 mL, 79 mmol), 2,8,9-triisobutyl-2,5,8,9-tetraza-1-phospha bicyclo{3.3.3}undecane (0.55 g, 1.6 mmol) and palladium (II) acetate (0.5 g, 2.1 mmol) in toluene (100 mL) and the reaction mixture was subjected to three cycles of evacuation-backfilling with argon in a Schlenk flask. The reaction mixture was heated at 80° C. for 13 hours. The mixture was filtered through Celite and concentrated to dryness. The residue was purified by silica gel column chromatography using 0-10% dichloromethane-methanol to give 3-fluoro-5-(4-methylpiperazin-1-yl)phenol (5.8 g, 35%) as an off-white solid. ESI-MS m/z calc. 210.12, found 211.2 (M+1). Retention time: 1.36 minutes. ¹H NMR (250 MHz, DMSO) δ (ppm): 9.74 (s, 1H), 6.31 (d, 1H), 6.19 (s, 1H), 6.07 (dd, 1H), 3.60-2.95 (br, 8H), 2.84 (s, 3H).

### Step 2: N-[4-[3-fluoro-5-(4-methylpiperazin-1-yl)phenoxy]-6-(o-tolyl)-5-(1,1,2,2,2-pentafluoroethyl)pyrimidin-2-yl]-1-methyl-pyrazole-4-sulfonamide

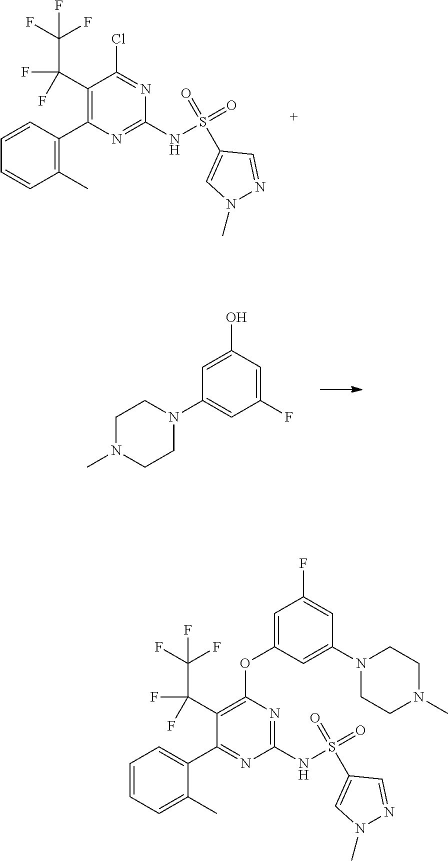 [SMILES: Cc1ccccc1-c1nc(NS(=O)(=O)c2cnn(C)c2)nc(Cl)c1C(F)(F)C(F)(F)F ; CN1CCN(c2cc(O)cc(F)c2)CC1 ; Cc1ccccc1-c1nc(NS(=O)(=O)c2cnn(C)c2)nc(Oc2cc(F)cc(N3CCN(C)CC3)c2)c1C(F)(F)C(F)(F)F]

To a 3-mL vial equipped with a magnetic stir bar, N-[4-chloro-6-(o-tolyl)-5-(1,1,2,2,2-pentafluoroethyl)pyrimidin-2-yl]-1-methyl-pyrazole-4-sulfonamide (16.30 mg, 0.03383 mmol), 3-fluoro-5-(4-methylpiperazin-1-yl)phenol (24.1 mg, 0.115 mmol), K₂CO₃ (21.5 mg, 0.156 mmol) and NMP (500 μL) were added. This slurry was stirred at 120° C. for 13 hours. The reaction mixture was then cooled to room temperature, diluted with MeOH (400 μL), filtered and purified by reverse phase HPLC (1-70% acetonitrile in water using HCl as a modifier) to give the desired product N-[4-[3-fluoro-5-(4-methylpiperazin-1-yl)phenoxy]-6-(o-tolyl)-5-(1,1,2,2,2-pentafluoroethyl)pyrimidin-2-yl]-1-methyl-pyrazole-4-sulfonamide (hydrochloride salt) (3.6 mg, 15%). ESI-MS m/z calc. 655.18005, found 656.4 (M+1)⁺; Retention time: 1.56 minutes; LC method A.

### Example 397: Preparation of Compound 1423

### Step 1: 2-(4-Methyl-piperazin-1-yl)-pyrimidin-5-ol

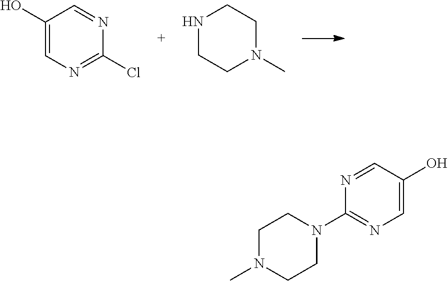

A mixture of 2-chloro-5-hydroxypyrimidine (40 g, 306.5 mmol) and N-methylpiperazine (200 mL, 1.8 mol) was stirred at 120° C. for 18 hours. The reaction mixture was concentrated under vacuum and the residue was recrystallized from acetonitrile to afford 2-(4-methyl-piperazin-1-yl)-pyrimidin-5-ol (42.4 g, 71.3% yield) as a beige solid. ESI-MS m/z calc. 194.12, found 195.0 (M+1). Retention time: 0.83 minutes. ¹H NMR (250 MHz, DMSO) δ (ppm): 8.02 (s, 2H), 3.55 (t, 4H), 2.33 (t, 4H), 2.19 (s, 3H).

### Step 2: 1-Methyl-N-[4-[2-(4-methylpiperazin-1-yl)pyrimidin-5-yl]oxy-6-(o-tolyl)-5-(1,1,2,2,2-pentafluoroethyl)pyrimidin-2-yl]pyrazole-4-sulfonamide

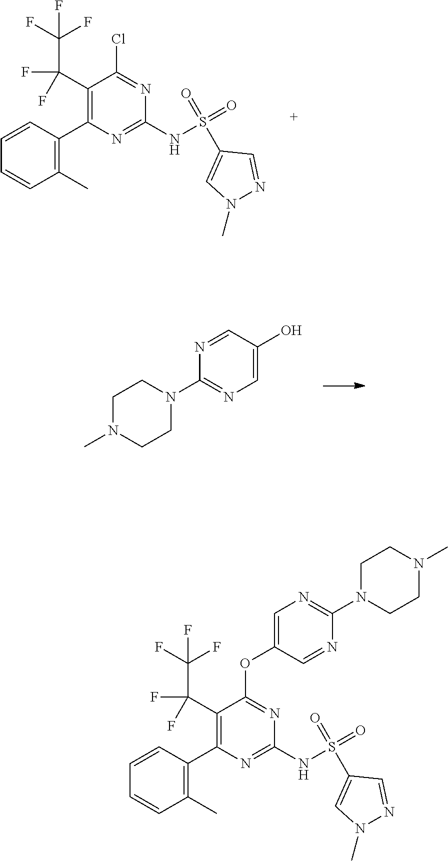 [SMILES: Cc1ccccc1-c1nc(NS(=O)(=O)c2cnn(C)c2)nc(Cl)c1C(F)(F)C(F)(F)F ; CN1CCN(c2ncc(O)cn2)CC1 ; Cc1ccccc1-c1nc(NS(=O)(=O)c2cnn(C)c2)nc(Oc2cnc(N3CCN(C)CC3)nc2)c1C(F)(F)C(F)(F)F]

To a 3-mL vial equipped with a magnetic stir bar, N-[4-chloro-6-(o-tolyl)-5-(1,1,2,2,2-pentafluoroethyl)pyrimidin-2-yl]-1-methyl-pyrazole-4-sulfonamide (16.5 mg, 0.03424 mmol), 2-(4-methylpiperazin-1-yl)pyrimidin-5-ol (21.4 mg, 0.1102 mmol), K₂CO₃ (25.0 mg, 0.1809 mmol) and NMP (500 μL) were added. This slurry was stirred at 120° C. for 19 hours. The reaction mixture was then cooled to room temperature, diluted with MeOH (400 μL), filtered and purified by reverse phase HPLC (1-70% acetonitrile in water using HCl as a modifier) to give a combined 7.1 mg of ˜₈₀% pure product and ˜₂₀% side product, N-[4-hydroxy-6-(o-tolyl)-5-(1,1,2,2,2-pentafluoroethyl)pyrimidin-2-yl]-1-methyl-pyrazole-4-sulfonamide. This mixture was re-purified by preparative TLC (one full silica plate, 20 cm×20 cm, 250 m thickness, 60 Å particle size, 10% methanol/EtOAc, UV active band at baseline) to give 1-methyl-N-[4-[2-(4-methylpiperazin-1-yl)pyrimidin-5-yl]oxy-6-(o-tolyl)-5-(1,1,2,2,2-pentafluoroethyl)pyrimidin-2-yl]pyrazole-4-sulfonamide (5.3 mg, 24%) ESI-MS m/z calc. 639.17993, found 640.4 (M+1)⁺; Retention time: 1.46 minutes. (LC method A).

### Example 398: Preparation of Compound 1424

### Step 1: N-[4-[2-chloro-3-(4-methylpiperazin-1-yl)phenoxy]-6-(2,6-dimethylphenyl)-5-(1,1,2,2,2-pentafluoroethyl)pyrimidin-2-yl]-1-methyl-pyrazole-4-sulfonamide

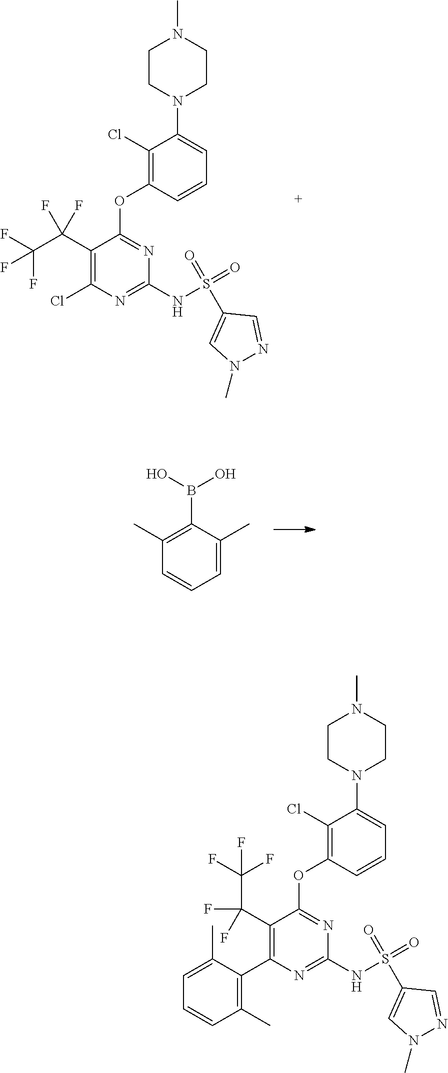

A heterogeneous solution of N-[4-chloro-6-[2-chloro-3-(4-methylpiperazin-1-yl)phenoxy]-5-(1,1,2,2,2-pentafluoroethyl)pyrimidin-2-yl]-1-methyl-pyrazole-4-sulfonamide (hydrochloride salt) (30.0 mg, 0.04595 mmol), (2,6-dimethylphenyl)boronic acid (13.8 mg, 0.09201 mmol), potassium carbonate (19.0 mg, 0.1375 mmol), and tetrakis(triphenylphosphine)palladium(0) (10.6 mg, 0.009173 mmol) in dioxane (200 μL) and water (40 μL) was microwaved in a sealed vial to 125° C. for 30 minutes

The reaction solution was acidified with acetic acid (50 μL, 0.8792 mmol), diluted with DMSO (0.5 mL) and filtered through a 0.45 m PTFE syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-[2-chloro-3-(4-methylpiperazin-1-yl)phenoxy]-6-(2,6-dimethylphenyl)-5-(1,1,2,2,2-pentafluoroethyl)pyrimidin-2-yl]-1-methyl-pyrazole-4-sulfonamide (hydrochloride salt) (0.50 mg, 2%) as a white solid. ESI-MS m/z calc. 685.16614, found 686.26 (M+1)⁺; Retention time: 1.64 minutes LC method A.

### Example 399: Preparation of Compound 1425

### Step 1: N-[4-[2-chloro-3-(4-methylpiperazin-1-yl)phenoxy]-6-(2-isopentylphenyl)-5-1,1,2,2,2-pentafluoroethyl)pyrimidin-2-yl]-1-methyl-pyrazole-4-sulfonamide

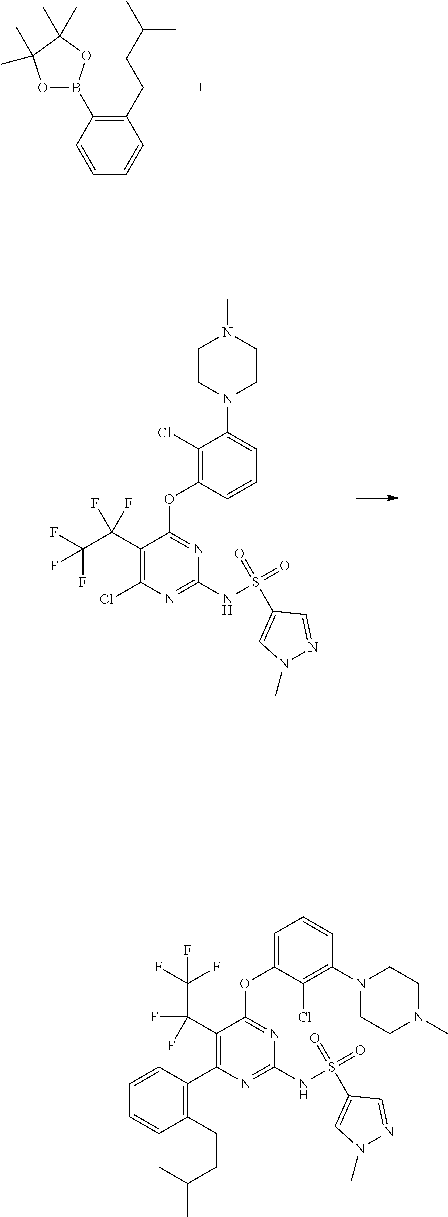 [SMILES: CC(C)CCc1ccccc1B1OC(C)(C)C(C)(C)O1 ; CN1CCN(c2cccc(Oc3nc(NS(=O)(=O)c4cnn(C)c4)nc(Cl)c3C(F)(F)C(F)(F)F)c2Cl)CC1 ; CC(C)CCc1ccccc1-c1nc(NS(=O)(=O)c2cnn(C)c2)nc(Oc2cccc(N3CCN(C)CC3)c2Cl)c1C(F)(F)C(F)(F)F]

A dioxane (0.5 mL) solution of 2-(2-isopentylphenyl)-4,4,5,5-tetramethyl-1,3,2-dioxaborolane (approximately 12.60 mg, 0.04595 mmol), N-[4-chloro-6-[2-chloro-3-(4-methylpiperazin-1-yl)phenoxy]-5-(1,1,2,2,2-pentafluoroethyl)pyrimidin-2-yl]-1-methyl-pyrazole-4-sulfonamide (hydrochloride salt) (20 mg, 0.03063 mmol), Pd(PPh₃)₄ (approximately 7.079 mg, 0.006126 mmol), and K₂CO₃ (approximately 61.25 μL of 2 M, 0.1225 mmol) was sparged with nitrogen for 30 seconds and then heated at 120° C. for 20 minutes. The mixture was diluted with MeOH (0.5 mL) and the solution was filtered and the filtrate purified by reverse phase chromatography using a 15 minutes gradient of 20% MeCN in water to 80% MeCN (HCl modifier) to give N-[4-[2-chloro-3-(4-methylpiperazin-1-yl)phenoxy]-6-(2-isopentylphenyl)-5-(1,1,2,2,2-pentafluoroethyl)pyrimidin-2-yl]-1-methyl-pyrazole-4-sulfonamide (hydrochloride salt) (5.6 mg, 24%). ESI-MS m/z calc. 727.2131, found 728.32 (M+1)⁺; Retention time: 1.76 minutes; LC method A.

### Example 400: Preparation of Compound 1426

### Step 1: N-[4-(2-isobutylphenyl)-6-(2-methylphenoxy)-5-(1,1,2,2,2-pentafluoroethyl)pyrimidin-2-yl]-1-methyl-pyrazole-4-sulfonamide

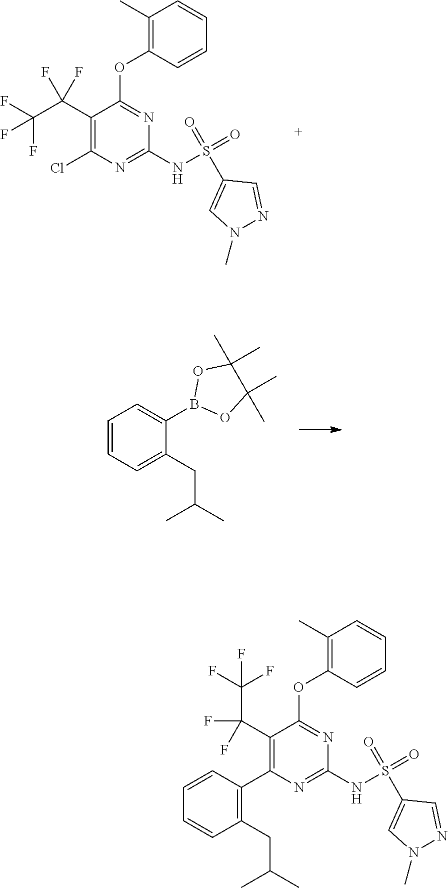 [SMILES: Cc1ccccc1Oc1nc(NS(=O)(=O)c2cnn(C)c2)nc(Cl)c1C(F)(F)C(F)(F)F ; CC(C)Cc1ccccc1B1OC(C)(C)C(C)(C)O1 ; Cc1ccccc1Oc1nc(NS(=O)(=O)c2cnn(C)c2)nc(-c2ccccc2CC(C)C)c1C(F)(F)C(F)(F)F]

A heterogeneous solution of N-[4-chloro-6-(2-methylphenoxy)-5-(1,1,2,2,2-pentafluoroethyl)pyrimidin-2-yl]-1-methyl-pyrazole-4-sulfonamide (30.0 mg, 0.06026 mmol), 2-(2-isobutylphenyl)-4,4,5,5-tetramethyl-1,3,2-dioxaborolane (31.4 mg, 0.1207 mmol), tetrakis(triphenylphosphine)palladium(0) (13.9 mg, 0.01203 mmol) and potassium carbonate (25.0 mg, 0.1809 mmol) in dioxane (250 μL) and water (50 μL) was microwaved in a sealed vial to 125° C. for 30 minutes. The reaction solution was acidified with acetic acid (70 μL, 1.231 mmol), diluted with DMSO (0.5 mL) and filtered through a 0.45 m PTFE syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-(2-isobutylphenyl)-6-(2-methylphenoxy)-5-(1,1,2,2,2-pentafluoroethyl)pyrimidin-2-yl]-1 -methyl-pyrazole-4-sulfonamide (4.5 mg, 13%) as a white solid. ESI-MS m/z calc. 595.16766, found 596.24 (M+1)⁺; Retention time: 2.33 minutes LC method A.

### Example 401: Characterization of Compounds 1427-1430

The compounds in the following tables were prepared in a manner analogous to that described above using commercially available reagents and intermediates described herein.

| | | LCMS | | | |
|---|---|---|---|---|---|
| | | Room | | | |
| Compound | | Temperature | Calc. | | LCMS |
| number | Structure | (min) | mass | M + 1 | Method |
| 1427 | 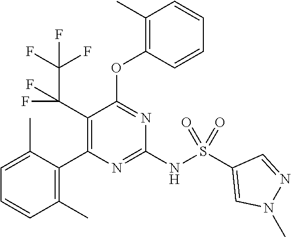 [SMILES: Cc1ccccc1Oc1nc(NS(=O)(=O)c2cnn(C)c2)nc(-c2c(C)cccc2C)c1C(F)(F)C(F)(F)F] | 2.18 | 567.136 | 568.22 | A |
| 1428 | 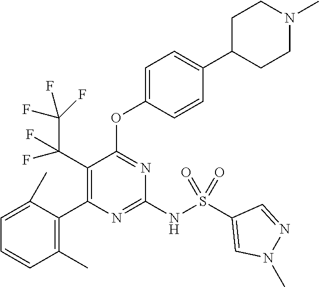 [SMILES: Cc1cccc(C)c1-c1nc(NS(=O)(=O)c2cnn(C)c2)nc(Oc2ccc(C3CCN(C)CC3)cc2)c1C(F)(F)C(F)(F)F] | 1.52 | 650.21 | 651.28 | A |
| 1429 | 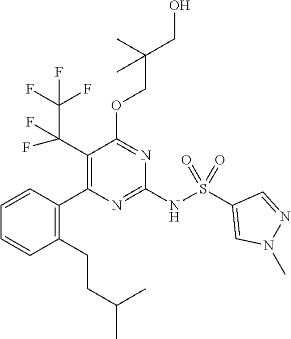 [SMILES: CC(C)CCc1ccccc1-c1nc(NS(=O)(=O)c2cnn(C)c2)nc(OCC(C)(C)CO)c1C(F)(F)C(F)(F)F] | 2.09 | 605.21 | 606.3 | A |
| 1430 | 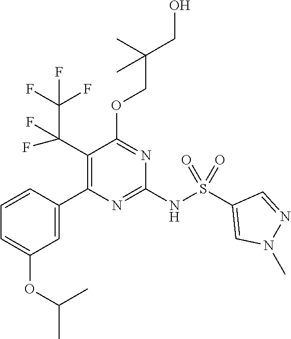 [SMILES: CC(C)Oc1cccc(-c2nc(NS(=O)(=O)c3cnn(C)c3)nc(OCC(C)(C)CO)c2C(F)(F)C(F)(F)F)c1] | 1.9 | 593.173 | 594.2 | A |

### Example 402: Preparation of Compound 1431

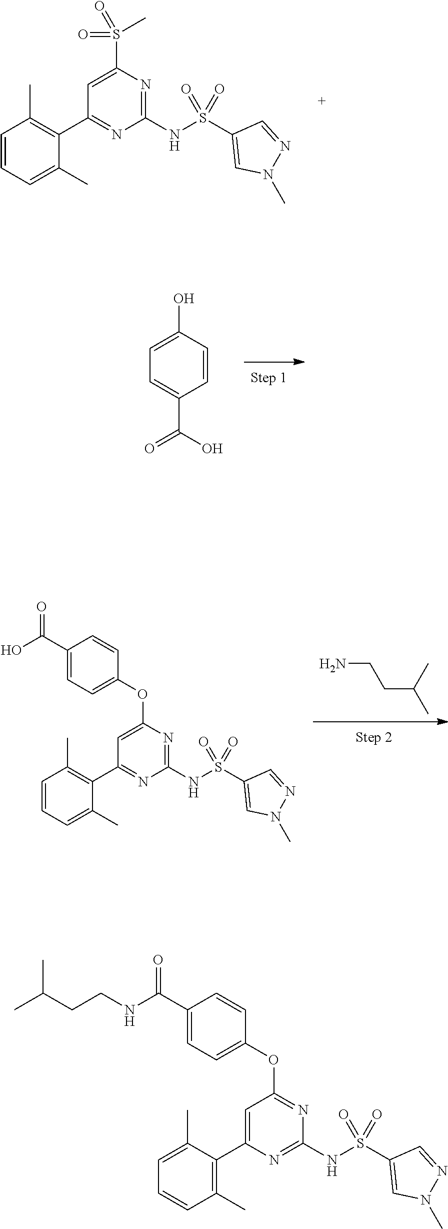

### Step 1: 4-[6-(2,6-Dimethylphenyl)-2-[(1-methylpyrazol-4-yl)sulfonylamino]pyrimidin-4-yl]oxybenzoic acid

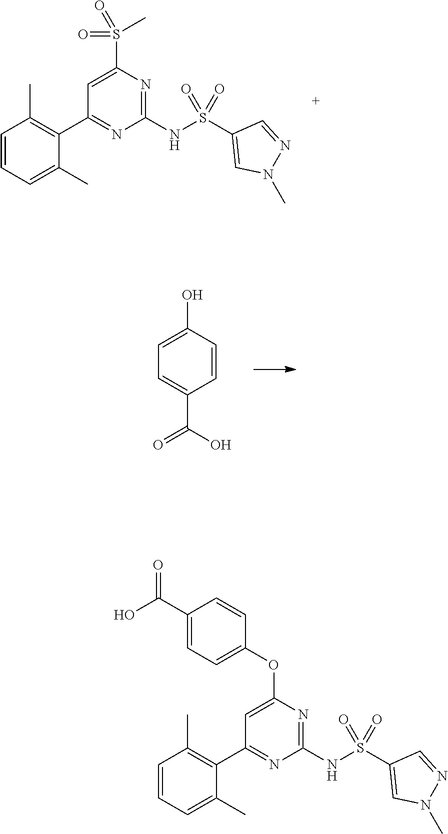 [SMILES: Cc1cccc(C)c1-c1cc(S(C)(=O)=O)nc(NS(=O)(=O)c2cnn(C)c2)n1 ; O=C(O)c1ccc(O)cc1 ; Cc1cccc(C)c1-c1cc(Oc2ccc(C(=O)O)cc2)nc(NS(=O)(=O)c2cnn(C)c2)n1]

To a solution of N-[4-(2,6-dimethylphenyl)-6-methylsulfonyl-pyrimidin-2-yl]-1 -methyl-pyrazole-4-sulfonamide (0.32 g, 0.7592 mmol) in NMP (6.400 mL) was added K₂CO₃ (0.53 g, 3.835 mmol) and 4-hydroxybenzoic acid (0.34 g, 2.462 mmol) and the reaction mixture stirred at 110° C. for 5 hours. The reaction mixture was poured into water and the pH brought to ˜4 with 1 N HCl and then extracted with EtOAc (3×). Organics were combined, washed with water (2×), brine, dried over Na₂SO₄ and evaporated to dryness. Purification by column chromatography (24 g silica; 0-75% EtOAc in hexanes) gave 4-[6-(2,6-dimethylphenyl)-2-[(1-methylpyrazol-4-yl)sulfonylamino]pyrimidin-4-yl]oxybenzoic acid (299 mg, 61%) as a white solid. ESI-MS m/z calc. 479.12634, found 480.2 (M+1)⁺; Retention time: 0.57 minutes; LC method D.

### Step 2: 4-[6-(2,6-Dimethylphenyl)-2-[(1-methylpyrazol-4-yl)sulfonylamino]pyrimidin-4-yl]oxy-N-isopentyl-benzamide

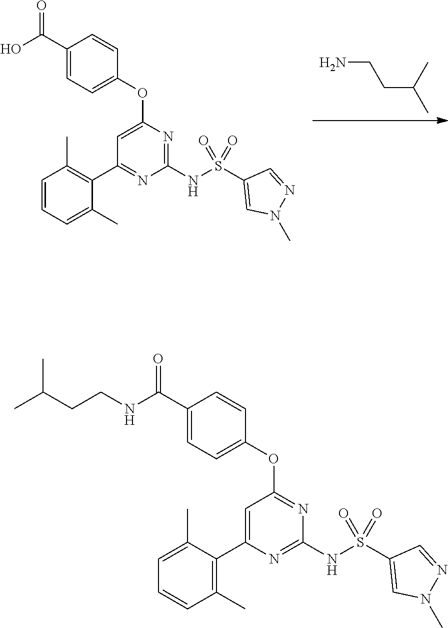

To a solution of 4-[6-(2,6-dimethylphenyl)-2-[(1-methylpyrazol-4-yl)sulfonylamino]pyrimidin-4-yl]oxybenzoic acid (25 mg, 0.03128 mmol), HATU (approximately 14.27 mg, 0.03754 mmol) and DIPEA (approximately 10.11 mg, 13.63 μL, 0.07820 mmol) was added 3-methylbutan-1-amine (approximately 5.453 mg, 7.261 μL, 0.06256 mmol) and the reaction mixture was stirred at room temperature for 16 hours. The reaction mixture was diluted with 1:1 DMSO:MeOH, filtered and purification by HPLC (1-99% ACN in water (HCl modifier)) gave 4-[6-(2,6-dimethylphenyl)-2-[(1-methylpyrazol-4-yl)sulfonylamino]pyrimidin-4-yl]oxy-N-isopentyl-benzamide (6.5 mg, 38%). ESI-MS m/z calc. 548.2206, found 549.2 (M+1)⁺; Retention time: 1.73 minutes; LC method A.

### Example 403: Preparation of Compound 1432 and Compound 1433

### Step 1: 4-[6-(2,6-Dimethylphenyl)-2-[(1-methylpyrazol-4-yl)sulfonylamino]pyrimidin-4-yl]oxy-3-methyl-benzoic acid

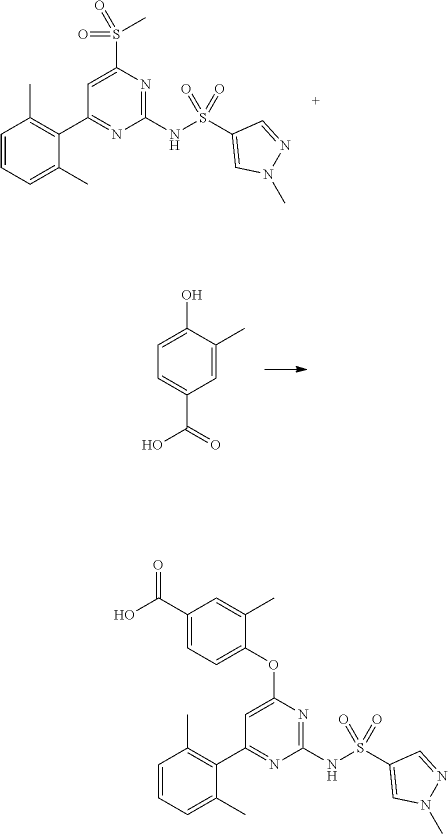 [SMILES: Cc1cccc(C)c1-c1cc(S(C)(=O)=O)nc(NS(=O)(=O)c2cnn(C)c2)n1 ; Cc1cc(C(=O)O)ccc1O ; Cc1cc(C(=O)O)ccc1Oc1cc(-c2c(C)cccc2C)nc(NS(=O)(=O)c2cnn(C)c2)n1]

To a solution of N-[4-(2,6-dimethylphenyl)-6-methylsulfonyl-pyrimidin-2-yl]-1 -methyl-pyrazole-4-sulfonamide (200 mg, 0.4745 mmol) in NMP (4.000 mL) was added 4-hydroxy-3-methyl-benzoic acid (219.7 mg, 1.444 mmol) and cesium carbonate (792.8 mg, 2.433 mmol) and the reaction mixture was stirred at 110° C. for 5 hours. The reaction mixture was poured into water and the pH brought to ˜4 with 1 N HCl and then extracted with EtOAc (3×). Organics were combined, washed with water (2×), brine, dried over sodium sulfate and evaporated to dryness. Purification by column chromatography (24 g silica; 0-5% MeOH in DCM) gave 4-[6-(2,6-dimethylphenyl)-2-[(1-methylpyrazol-4-yl)sulfonylamino]pyrimidin-4-yl]oxy-3-methyl-benzoic acid (142 mg, 61%) as a white solid. ESI-MS m/z calc. 493.142, found 495.48 (M+1)⁺; Retention time: 1.52 minutes; LC method A.

### Step 2: 4-[6-(2,6-Dimethylphenyl)-2-[(1-methylpyrazol-4-yl)sulfonylamino]pyrimidin-4-yl]oxy-N-(2-methoxyethyl)-3-methyl-benzamide

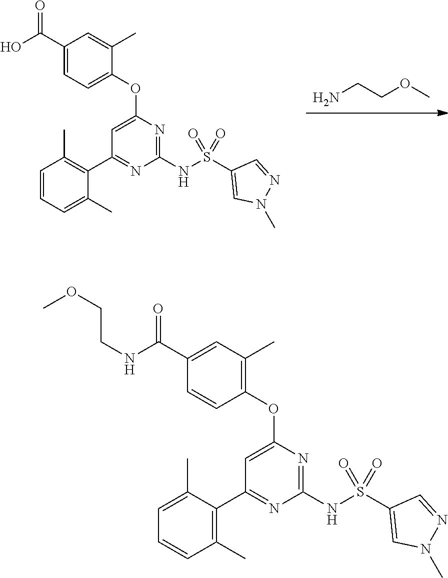

To a solution of 4-[6-(2,6-dimethylphenyl)-2-[(1-methylpyrazol-4-yl)sulfonylamino]pyrimidin-4-yl]oxy-3-methyl-benzoic acid (15 mg, 0.03039 mmol) in NMP (0.5 mL) was added 2-methoxyethanamine (approximately 2.283 mg, 2.642 μL, 0.03039 mmol) and DIPEA (approximately 3.928 mg, 5.294 μL, 0.03039 mmol). Propylphosphonic anhydride (T3P, 50 wt % anhydride solution in ethylacate) (approximately 19.34 μL of 50% w/v, 0.03039 mmol) was added and the reaction was stirred for 1 hour. The reaction mixture was diluted DMSO and purification by HPLC (1-99% ACN in water (HCl modifier)) gave 4-[6-(2,6-dimethylphenyl)-2-[(1-methylpyrazol-4-yl)sulfonylamino]pyrimidin-4-yl]oxy-N-(2-methoxyethyl)-3-methyl-benzamide (8.9 mg, 53%). ESI-MS m/z calc. 550.1998, found 552.48 (M+1)⁺; Retention time: 1.48 minutes; LC method A.

### Example 404: Preparation of Compound 1434

### Step 1: 2-Chloro-4-[6-(2,6-dimethylphenyl)-2-[(1-methylpyrazol-4-yl)sulfonylamino]pyrimidin-4-yl]oxy-benzoic acid

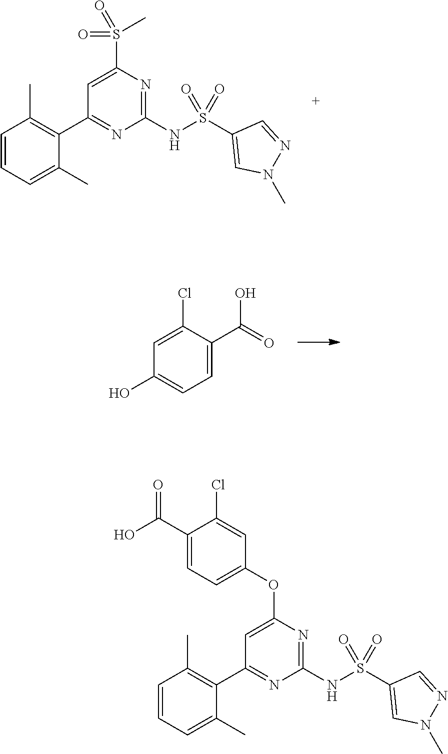 [SMILES: Cc1cccc(C)c1-c1cc(S(C)(=O)=O)nc(NS(=O)(=O)c2cnn(C)c2)n1 ; O=C(O)c1ccc(O)cc1Cl ; Cc1cccc(C)c1-c1cc(Oc2ccc(C(=O)O)c(Cl)c2)nc(NS(=O)(=O)c2cnn(C)c2)n1]

To a solution of N-[4-(2,6-dimethylphenyl)-6-methylsulfonyl-pyrimidin-2-yl]-1 -methyl-pyrazole-4-sulfonamide (400 mg, 0.9490 mmol) in NMP (8 mL) was added 2-chloro-4-hydroxy-benzoic acid (approximately 491.3 mg, 2.847 mmol) and cesium carbonate (approximately 1.546 g, 4.745 mmol). The reaction mixture stirred at 110° C. for 5 hours. The reaction mixture was poured into water and the pH brought to ˜4 with 1 N HCl and then extracted with EtOAc (3×). Organics were combined, washed with water (2×), brine, dried over sodium sulfate and evaporated to dryness. Purification by column chromatography (40 g silica; 0-5% MeOH in DCM) gave 2-chloro-4-[6-(2,6-dimethylphenyl)-2-[(1-methylpyrazol-4-yl)sulfonylamino]pyrimidin-4-yl]oxy-benzoic acid (270 mg, 55%) as a white solid. ESI-MS m z calc. 513.08734, found 515.4 (M+1)⁺; Retention time: 1.53 minutes; LC method A.

### Step 2: 2-Chloro-4-[6-(2,6-dimethylphenyl)-2-[(1-methylpyrazol-4-yl)sulfonylamino]pyrimidin-4-yl]oxy-N-(2-methoxyethyl)benzamide

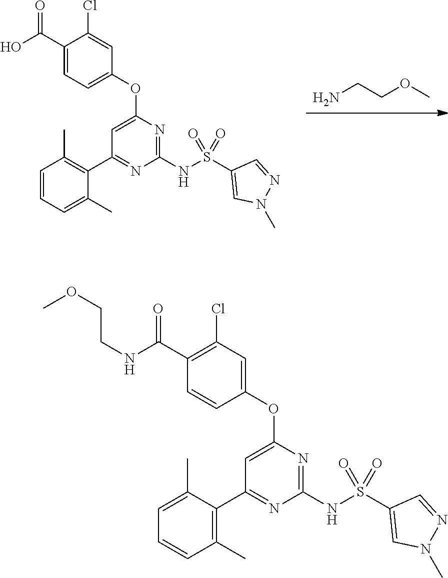

The compound was prepared in a manner analogous to that described above using commercially available 2-methoxyethanamine to give 2-chloro-4-[6-(2,6-dimethylphenyl)-2-[(1-methylpyrazol-4-yl)sulfonylamino]pyrimidin-4-yl]oxy-N-(2-methoxyethyl)benzamide (13 mg, 78%). ESI-MS m/z calc. 570.1452, found 572.55 (M+1)⁺; Retention time: 1.49 minutes; LC method A.

### Example 405: Preparation of Compound 1435

### Step 1: 3-Chloro-5-[6-(2,6-dimethylphenyl)-2-[(1-methylpyrazol-4-yl)sulfonylamino]pyrimidin-4-yl]oxy-benzoic acid

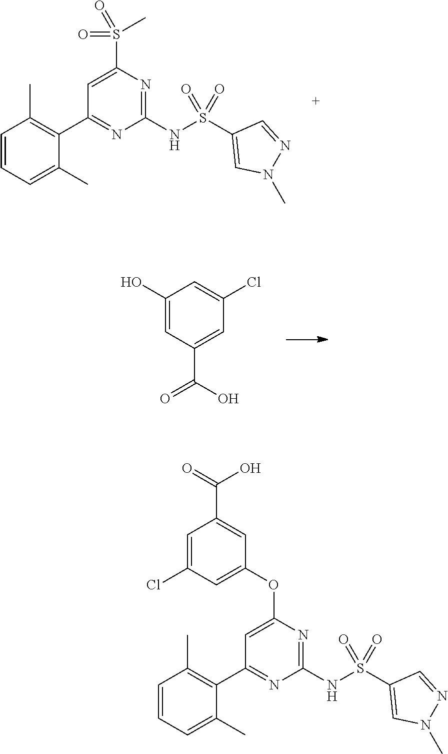 [SMILES: Cc1cccc(C)c1-c1cc(S(C)(=O)=O)nc(NS(=O)(=O)c2cnn(C)c2)n1 ; O=C(O)c1cc(O)cc(Cl)c1 ; Cc1cccc(C)c1-c1cc(Oc2cc(Cl)cc(C(=O)O)c2)nc(NS(=O)(=O)c2cnn(C)c2)n1]

To a solution of N-[4-(2,6-dimethylphenyl)-6-methylsulfonyl-pyrimidin-2-yl]-1 -methyl-pyrazole-4-sulfonamide (175 mg, 0.4152 mmol) in NMP (3 mL) was added 3-chloro-5-hydroxy-benzoic acid (205.0 mg, 1.188 mmol) and cesium carbonate (696.4 mg, 2.137 mmol). The reaction mixture was stirred at 110° C. for 3 hours. The reaction mixture was poured into water and the pH brought to ˜4 with 1 N HCl and then extracted with EtOAc (3×). Organics were combined, washed with water (2×), brine, dried over sodium sulfate and evaporated to dryness. Purification by column chromatography (40 g silica; 0-5% MeOH in DCM) gave 3-chloro-5-[6-(2,6-dimethylphenyl)-2-[(1-methylpyrazol-4-yl)sulfonylamino]pyrimidin-4-yl]oxy-benzoic acid (129 mg, 60%) as white solid. ESI-MS m/z calc. 513.08734, found 515.37 (M+1)⁺; Retention time: 1.62 minutes; LC method A.

### Step 2: 3-Chloro-5-[6-(2,6-dimethylphenyl)-2-[(1-methylpyrazol-4-yl)sulfonylamino]pyrimidin-4-yl]oxy-N-methyl-benzamide

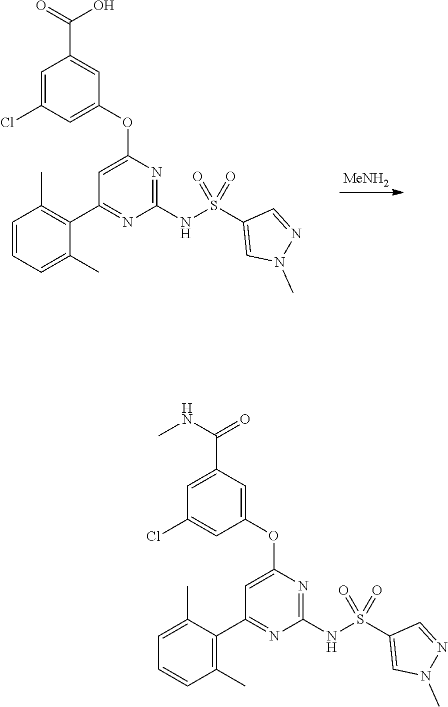 [SMILES: "MeNH2" ; Cc1cccc(C)c1-c1cc(Oc2cc(Cl)cc(C(=O)O)c2)nc(NS(=O)(=O)c2cnn(C)c2)n1 ; CNC(=O)c1cc(Cl)cc(Oc2cc(-c3c(C)cccc3C)nc(NS(=O)(=O)c3cnn(C)c3)n2)c1]

The compound was prepared in a manner analogous to that described above using commercially available methyl amine to give 3-chloro-5-[6-(2,6-dimethylphenyl)-2-[(1-methylpyrazol-4-yl)sulfonylamino]pyrimidin-4-yl]oxy-N-methyl-benzamide (11 mg, 72%). ESI-MS m/z calc. 526.119, found 526.59 (M+1)⁺; Retention time: 1.58 minutes; LC method A.

### Example 406: Preparation of Compound 1436

### Step 1: 3-[6-(2,6-Dimethylphenyl)-2-[(1-methylpyrazol-4-yl)sulfonylamino]pyrimidin-4-yl]oxy-4-methyl-benzoic acid

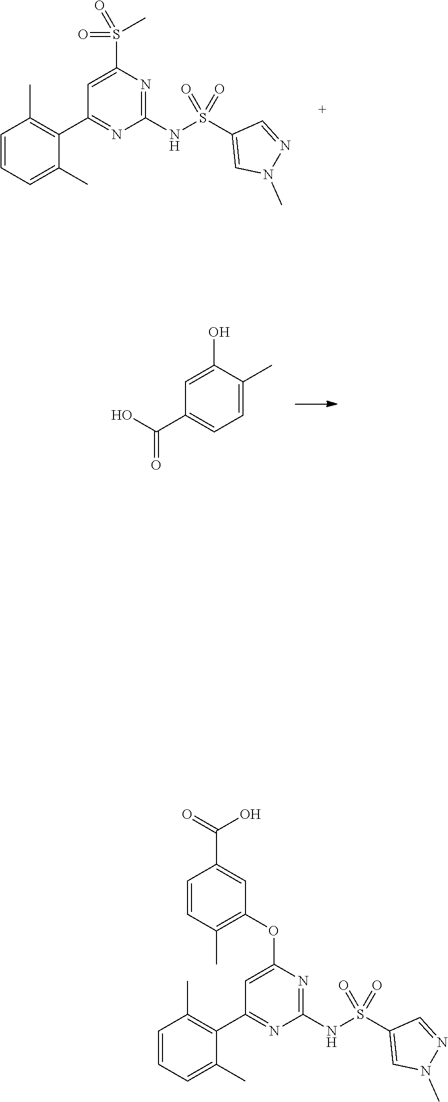 [SMILES: Cc1cccc(C)c1-c1cc(S(C)(=O)=O)nc(NS(=O)(=O)c2cnn(C)c2)n1 ; Cc1ccc(C(=O)O)cc1O ; Cc1ccc(C(=O)O)cc1Oc1cc(-c2c(C)cccc2C)nc(NS(=O)(=O)c2cnn(C)c2)n1]

The compound was prepared in a manner analogous to that described above using commercially available -hydroxy-4-methyl-benzoic acid to give 3-[6-(2,6-dimethylphenyl)-2-[(1-methylpyrazol-4-yl)sulfonylamino]pyrimidin-4-yl]oxy-4-methyl-benzoic acid (160 mg, 68%) as a white solid. ESI-MS m/z calc. 493.142, found 495.41 (M+1)⁺; Retention time: 1.51 minutes; LC method A.

### Step 2: 3-[6-(2,6-Dimethylphenyl)-2-[(1-methylpyrazol-4-yl)sulfonylamino]pyrimidin-4-yl]oxy-4-methyl-N-propyl-benzamide

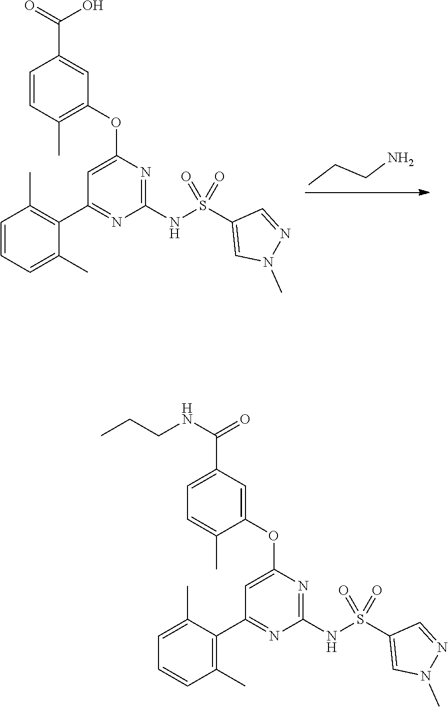

The compound was prepared in a manner analogous to that described above using commercially available propan-1-amine to give 3-[6-(2,6-dimethylphenyl)-2-[(1-methylpyrazol-4-yl)sulfonylamino]pyrimidin-4-yl]oxy-4-methyl-N-propyl-benzamide (13.2 mg, 81%). ESI-MS m/z calc. 534.2049, found 535.27 (M+1)⁺; Retention time: 1.62 minutes; LC method A.

### Example 407: Preparation of Compound 1437

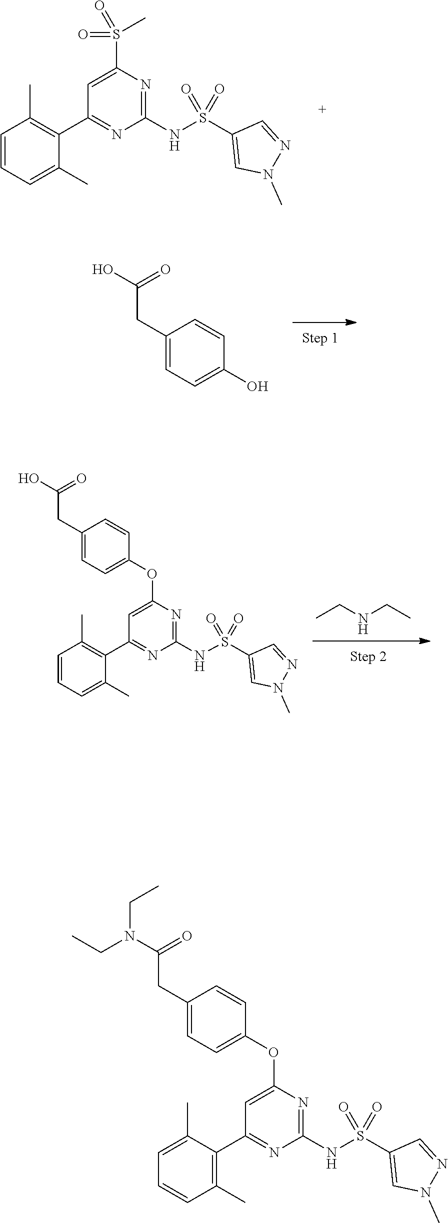

### Step 1: 2-[4-[6-(2,6-Dimethylphenyl)-2-[(1-methylpyrazol-4-yl)sulfonylamino]pyrimidin-4-yl]oxyphenyl]acetic acid

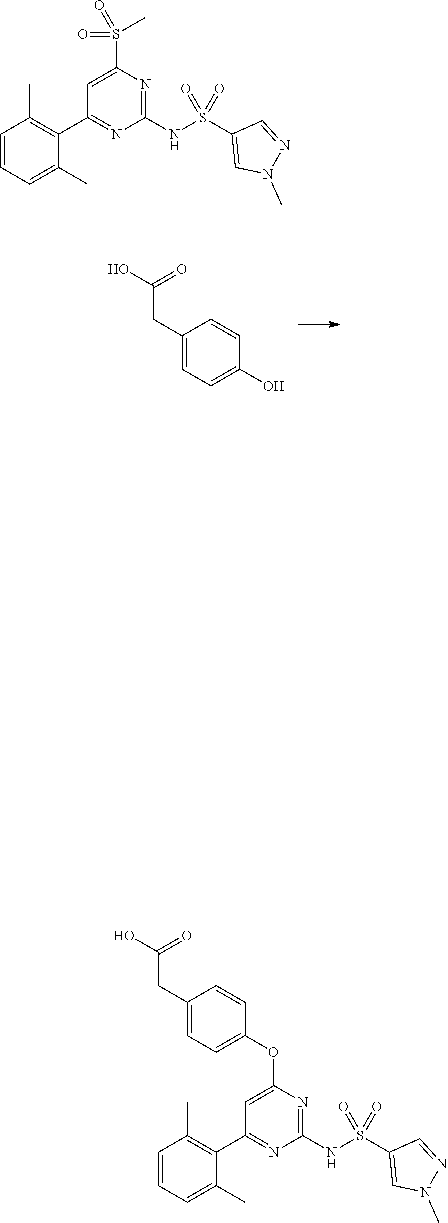 [SMILES: Cc1cccc(C)c1-c1cc(S(C)(=O)=O)nc(NS(=O)(=O)c2cnn(C)c2)n1 ; O=C(O)Cc1ccc(O)cc1 ; Cc1cccc(C)c1-c1cc(Oc2ccc(CC(=O)O)cc2)nc(NS(=O)(=O)c2cnn(C)c2)n1]

To a 20 mL vial equipped with a magnetic stir bar, N-[4-(2,6-dimethylphenyl)-6-methylsulfonyl-pyrimidin-2-yl]-1-methyl-pyrazole-4-sulfonamide (102.3 mg, 0.2427 mmol), N-methylpyrrolidinone (4.0 mL) and 2-(4-hydroxyphenyl)acetic acid (101.1 mg, 0.6645 mmol) were added, followed by potassium carbonate (132.3 mg, 0.9573 mmol). This mixture was stirred at 150° C. for 2 hours. The reaction mixture was then cooled to room temperature, quenched with 1 N HCl (5 mL), and extracted with ethyl acetate (3×7 mL). The combined organic extracts was washed with water (2×10 mL) and saturated aqueous sodium chloride solution (10 mL), then dried over sodium sulfate, filtered, and evaporated in vacuo. This crude material was purified by silica gel chromatography (4 g of silica, 0 to 40% gradient of ethyl acetate/hexanes) to give 2-[4-[6-(2,6-dimethylphenyl)-2-[(1-methylpyrazol-4-yl)sulfonylamino]pyrimidin-4-yl]oxyphenyl]acetic acid (105.0 mg, 88%) ESI-MS m/z calc. 493.142, found 494.2 (M+1)⁺; Retention time: 0.56 minutes, LC method D.

### Step 2: 2-[4-[6-(2,6-Dimethylphenyl)-2-[(1-methylpyrazol-4-yl)sulfonylamino]pyrimidin-4-yl]oxyphenyl]-N,N-diethyl-acetamide

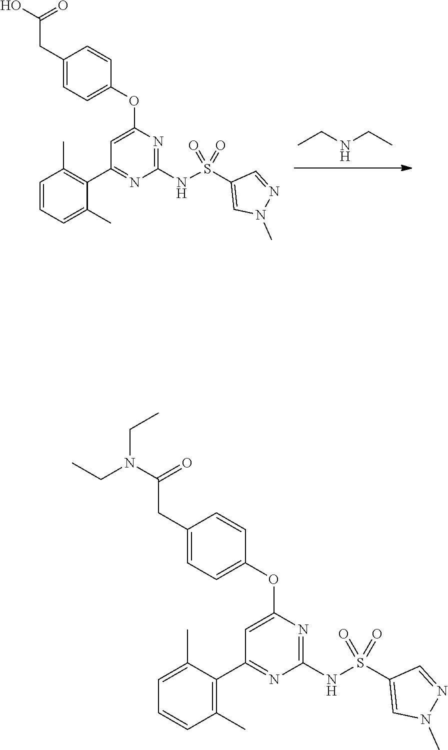

To a 3 mL vial equipped with a magnetic stir bar, 2-[4-[6-(2,6-dimethylphenyl)-2-[(1-methylpyrazol-4-yl)sulfonylamino]pyrimidin-4-yl]oxyphenyl]acetic acid (16.70 mg, 0.03384 mmol) (16.7 mg, 0.0338 mmol), DMF (1.0 mL), N-ethylethanamine (hydrochloride salt) (10.96 mg, 15.50 μL, 0.1 mmol), DIPEA (18.55 mg, 25.00 μL, 0.1435 mmol) (25 μL, 0.14 mmol) and HATU (38.00 mg, 0.09995 mmol) (38.0 mg, 0.100 mmol) were added, in this order. This mixture was stirred at room temperature for 5 minutes, after which it was filtered and purified by reverse phase HPLC (1-70% acetonitrile in water using HCl as a modifier) to give the desired product 2-[4-[6-(2,6-dimethylphenyl)-2-[(1-methylpyrazol-4-yl)sulfonylamino]pyrimidin-4-yl]oxyphenyl]-N,N-diethyl-acetamide (10.0 mg, 54%). ESI-MS m/z calc. 548.2206, found 549.3 (M+1)⁺; Retention time: 1.61 minutes; LC method A.

### Example 408: Preparation of Compound 1438

### Step 1: N-[4-(2,6-Dimethylphenyl)-6-[4-[2-oxo-2-(1-piperidyl)ethyl]phenoxy]pyrimidin-2-yl]-1-methyl-pyrazole-4-sulfonamide

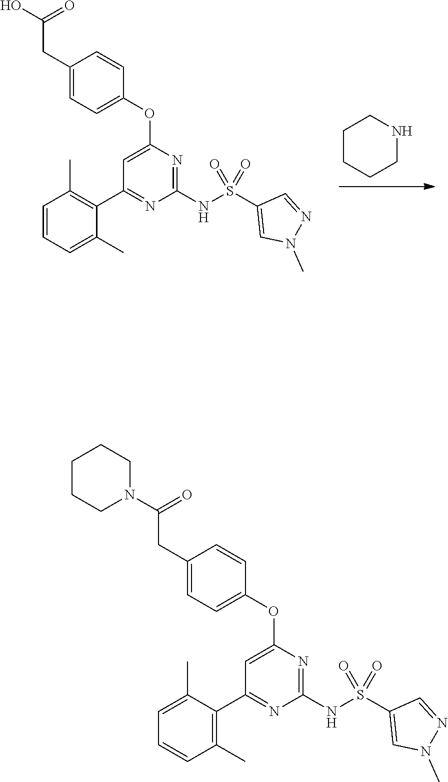 [SMILES: Cc1cccc(C)c1-c1cc(Oc2ccc(CC(=O)O)cc2)nc(NS(=O)(=O)c2cnn(C)c2)n1 ; C1CCNCC1 ; Cc1cccc(C)c1-c1cc(Oc2ccc(CC(=O)N3CCCCC3)cc2)nc(NS(=O)(=O)c2cnn(C)c2)n1]

The compound was prepared in a manner analogous to that described above using commercially available piperidine to give N-[4-(2,6-dimethylphenyl)-6-[4-[2-oxo-2-(1-piperidyl)ethyl]phenoxy]pyrimidin-2-yl]-1-methyl-pyrazole-4-sulfonamide (7 mg, 37%). ESI-MS m/z calc. 560.2206, found 561.4 (M+1)⁺; Retention time: 1.65 minutes; LC method A.

### Example 409: Preparation of Compound 1439

### Step 1: 4-[6-(2,6-Dimethylphenyl)-2-[(1-methylpyrazol-4-yl)sulfonylamino]pyrimidin-4-yl]oxy-N,3-dimethyl-benzamide

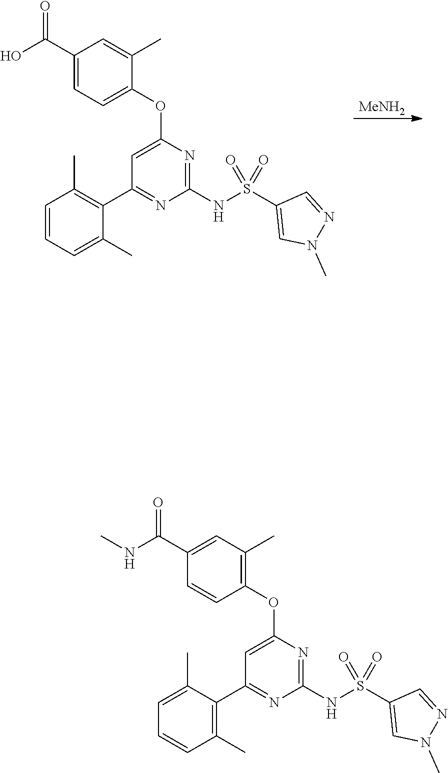 [SMILES: "MeNH2" ; Cc1cc(C(=O)O)ccc1Oc1cc(-c2c(C)cccc2C)nc(NS(=O)(=O)c2cnn(C)c2)n1 ; CNC(=O)c1ccc(Oc2cc(-c3c(C)cccc3C)nc(NS(=O)(=O)c3cnn(C)c3)n2)c(C)c1]

The compound was prepared in a manner analogous to that described above using commercially available methylamine to give 4-[6-(2,6-dimethylphenyl)-2-[(1-methylpyrazol-4-yl)sulfonylamino]pyrimidin-4-yl]oxy-N,3-dimethyl-benzamide (8.2 mg, 53%). ESI-MS m z calc. 506.1736, found 508.5 (M+1)⁺; Retention time: 1.42 minutes; LC method A.

### Example 410: Preparation of Compound 1440

### Step 1: 3-[6-(2,6-Dimethylphenyl)-2-[(1-methylpyrazol-4-yl)sulfonylamino]pyrimidin-4-yl]oxy-N-methyl-benzamide

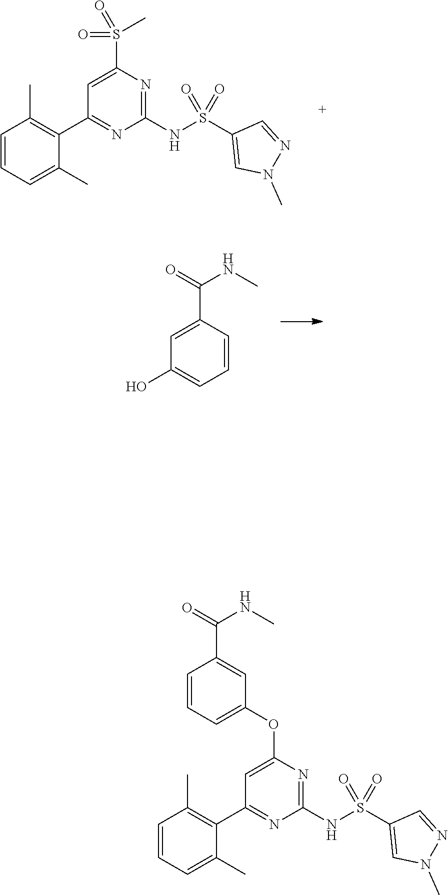 [SMILES: Cc1cccc(C)c1-c1cc(S(C)(=O)=O)nc(NS(=O)(=O)c2cnn(C)c2)n1 ; CNC(=O)c1cccc(O)c1 ; CNC(=O)c1cccc(Oc2cc(-c3c(C)cccc3C)nc(NS(=O)(=O)c3cnn(C)c3)n2)c1]

A suspension of N-[4-(2,6-dimethylphenyl)-6-methylsulfonyl-pyrimidin-2-yl]-1 -methyl-pyrazole-4-sulfonamide (20 mg, 0.04745 mmol), 3-hydroxy-N-methyl-benzamide (16 mg, 0.1058 mmol) and K₂CO₃ (22 mg, 0.1592 mmol) was stirred in NMP (0.4 mL) at 80° C. for 16 hours. The reaction mixture was diluted with MeOH, filtered and purification by HPLC (1-99% ACN in water (HCl modifier)) gave 3-[6-(2,6-dimethylphenyl)-2-[(1-methylpyrazol-4-yl)sulfonylamino]pyrimidin-4-yl]oxy-N-methyl-benzamide as a foam. ESI-MS m/z calc. 492.15796, found 493.2 (M+1)⁺; Retention time: 1.34 minutes; LC method A.

### Example 411: Preparation of Compound 1441

### Step 1: 4-[6-(2,6-Dimethylphenyl)-2-[(1-methylpyrazol-4-yl)sulfonylamino]pyrimidin-4-yl]oxy-N,N-diethyl-3-methyl-benzamide

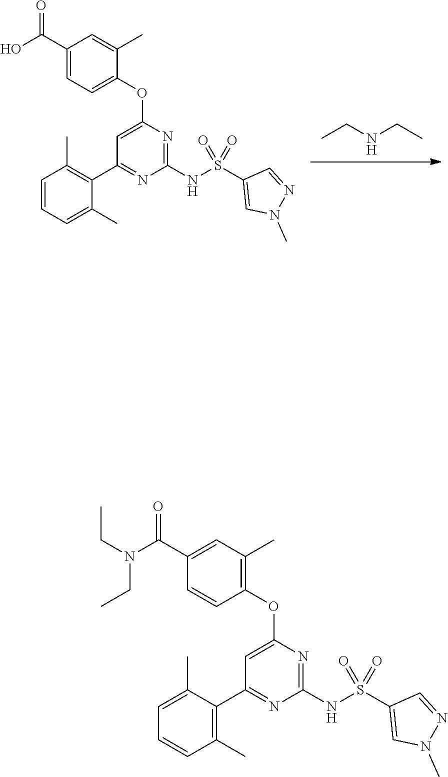

The compound was prepared in a manner analogous to that described above using commercially available N-ethylethanamine to give 4-[6-(2,6-dimethylphenyl)-2-[(1-methylpyrazol-4-yl)sulfonylamino]pyrimidin-4-yl]oxy-N,N-diethyl-3-methyl-benzamide (9.6 mg, 58%). ESI-MS m/z calc. 548.2206, found 550.49 (M+1)⁺; Retention time: 1.7 minutes; LC method A.

### Example 412: Preparation of Compound 1442

### Step 1: 4-[6-(2,6-Dimethylphenyl)-2-[(1-methylpyrazol-4-yl)sulfonylamino]pyrimidin-4-yl]oxy-N,N-diethyl-benzamide

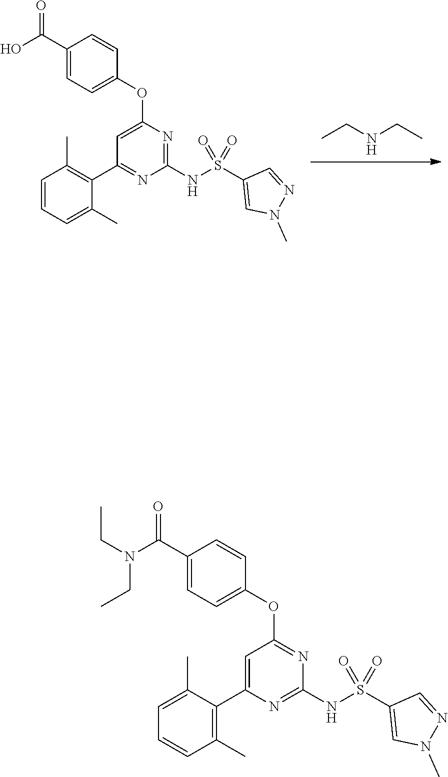

The compound was prepared in a manner analogous to that described above using commercially available N-ethylethanamine to give 4-[6-(2,6-dimethylphenyl)-2-[(1-methylpyrazol-4-yl)sulfonylamino]pyrimidin-4-yl]oxy-N,N-diethyl-benzamide (6.6 mg, 39%). ESI-MS m/z calc. 534.2049, found 535.06 (M+1)⁺; Retention time: 1.6 minutes; LC method A.

### Example 413: Preparation of Compound 1443

### Step 1: N-[4-(2,6-dimethylphenyl)-6-[4-(2-dimethylphosphorylethyl)phenoxy]pyrimidin-2-yl]-1-methyl-pyrazole-4-sulfonamide

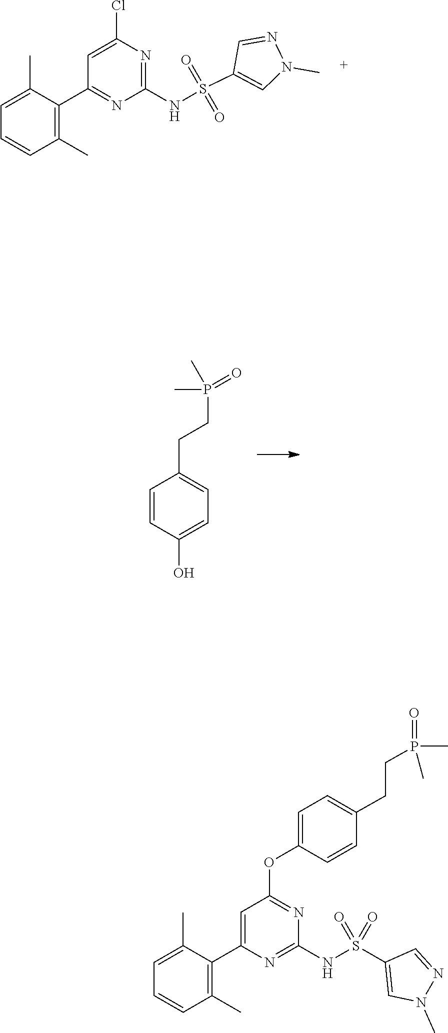 [SMILES: Cc1cccc(C)c1-c1cc(Cl)nc(NS(=O)(=O)c2cnn(C)c2)n1 ; CP(C)(=O)CCc1ccc(O)cc1 ; Cc1cccc(C)c1-c1cc(Oc2ccc(CCP(C)(C)=O)cc2)nc(NS(=O)(=O)c2cnn(C)c2)n1]

A heterogeneous solution of 4-(2-dimethylphosphorylethyl)phenol (13.2 mg, 0.06660 mmol), N-[4-chloro-6-(2,6-dimethylphenyl)pyrimidin-2-yl]-1-methyl-pyrazole-4-sulfonamide (21 mg, 0.05558 mmol), and potassium carbonate (23.0 mg, 0.1664 mmol) in NMP (110 μL) was heated in a sealed vial to 150° C. for 5 hours. The solution was cooled, acidified with acetic acid (33.4 mg, 0.5562 mmol) and diluted with DMSO (0.5 mL). The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-(2,6-dimethylphenyl)-6-[4-(2-dimethylphosphorylethyl)phenoxy]pyrimidin-2-yl]-1-methyl-pyrazole-4-sulfonamide (4.9 mg, 16%) as a white solid. ESI-MS m/z calc. 539.1756, found 540.28 (M+1)⁺; Retention time: 1.36 minutes; LC method A.

### Example 414: Preparation of Compound 1444

### Step 1: N-[4-(4-bromophenoxy)-6-(2,6-dimethylphenyl)pyrimidin-2-yl]-1-methyl-pyrazole-4-sulfonamide

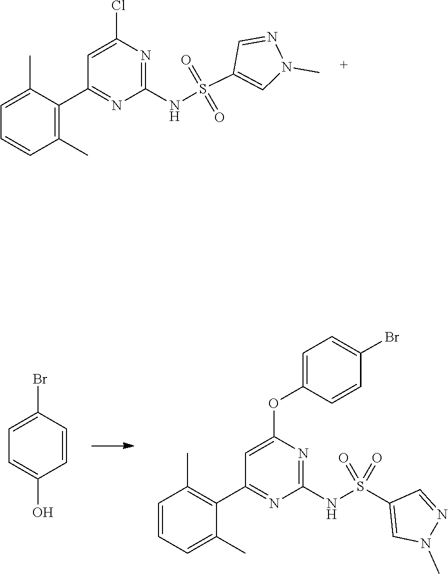 [SMILES: Cc1cccc(C)c1-c1cc(Cl)nc(NS(=O)(=O)c2cnn(C)c2)n1 ; Oc1ccc(Br)cc1 ; Cc1cccc(C)c1-c1cc(Oc2ccc(Br)cc2)nc(NS(=O)(=O)c2cnn(C)c2)n1]

An NMP (12 mL) solution of 4-bromophenol (approximately 1.483 g, 8.574 mmol), cesium carbonate (approximately 931.2 mg, 2.858 mmol), and N-[4-chloro-6-(2,6-dimethylphenyl)pyrimidin-2-yl]-1-methyl-pyrazole-4-sulfonamide (1.08 g, 2.858 mmol) was heated to 110° C. for 16 hours and then diluted with ethyl acetate (50 mL) and water (20 mL). The pH of aqueous layer was adjusted to 7-8 by the addition of concentrated HCl dropwise via syringe. The product was extracted with ethyl acetate (25 mL×2). The organic layers were combined and washed with water (5 mL×5) and then dried over anhydrous sodium sulfate, filtered, and concentrated in vacuo and chased with diethyl ether (10 mL) to −2 mL. Then 5 mL of diethyl ether was added, and the mixture was stirred at room temperature for 1 hour upon which the product crystallized out as a white slurry. It was cooled to 0° C. and stirred for 30 minutes. The solid was filtered and washed with cool diethyl ether (1 mL) and dried under high vacuum to give N-[4-(4-bromophenoxy)-6-(2,6-dimethylphenyl)pyrimidin-2-yl]-1-methyl-pyrazole-4-sulfonamide (1.40 g, 95%). ESI-MS m/z calc. 513.047, found 516.36 (M+1)⁺; Retention time: 1.79 minutes; LCMS Method: (LC method A).

### Step 2: N-[4-(2,6-dimethylphenyl)-6-(4-dimethylphosphorylphenoxy)pyrimidin-2-yl]-1-methyl-pyrazole-4-sulfonamide

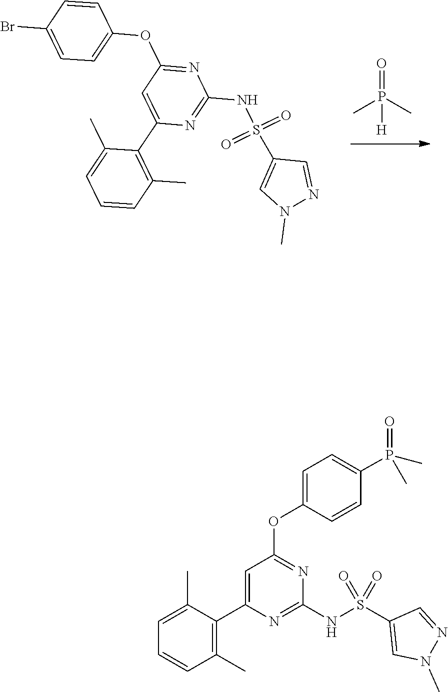

A heterogeneous solution of N-[4-(4-bromophenoxy)-6-(2,6-dimethylphenyl)pyrimidin-2-yl]-1-methyl-pyrazole-4-sulfonamide (25.0 mg, 0.04860 mmol), methylphosphonoylmethane (7.6 mg, 0.09737 mmol), potassium carbonate (20.2 mg, 0.146 mmol) and copper(I) iodide (0.5 mg, 0.003 mmol) in toluene (160 μL) and DMF (160 μL) was degassed by sonicating for 5 minutes. The reaction vessel was sealed and microwaved at 120° C. for 40 minutes. The reaction was diluted with DMSO (0.50 mL) and acidified with acetic acid (55 μL, 0.97 mmol). The crude reaction mixture was filtered and separated by HPLC (acetonitrile in water with 0.1% hydrochloric acid). N-[4-(2,6-dimethylphenyl)-6-(4-dimethylphosphorylphenoxy)pyrimidin-2-yl]-1-methyl-pyrazole-4-sulfonamide (11.5 mg, 46%) was isolated as a white solid. ESI-MS m/z calc. 511.14432, found 512.48 (M+1)⁺; Retention time: 1.28 minutes; LC method A.

### Example 415: Preparation of Compound 1445

### Step 1: N-[4-(2,6-Dimethylphenyl)-6-(2-fluorophenoxy)pyrimidin-2-yl]-1-methyl-pyrazole-4-sulfonamide

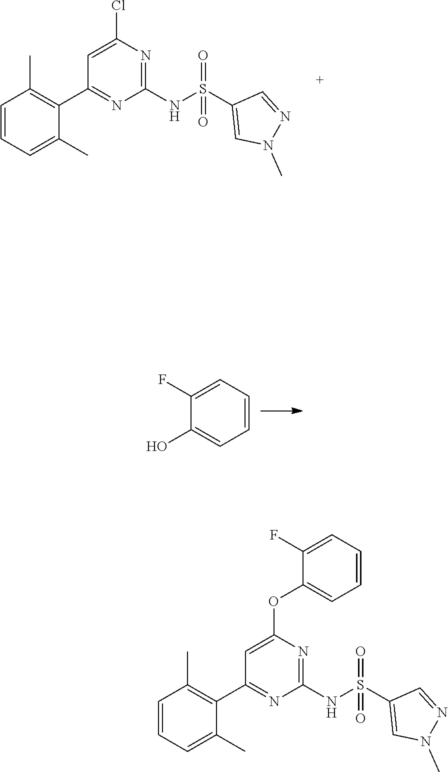 [SMILES: Cc1cccc(C)c1-c1cc(Cl)nc(NS(=O)(=O)c2cnn(C)c2)n1 ; Oc1ccccc1F ; Cc1cccc(C)c1-c1cc(Oc2ccccc2F)nc(NS(=O)(=O)c2cnn(C)c2)n1]

Into a 20 mL glass vial was added N-[4-chloro-6-(2,6-dimethylphenyl)pyrimidin-2-yl]-1-methyl-1H-pyrazole-4-sulfonamide (227 mg, 0.6 mmol), 2-fluorophenol (0.065 mL, 0.72 mmol) and potassium carbonate (166 mg, 1.2 mmol) in acetonitrile (6 mL). The reaction mixture was heated at 80° C. for 16 hours. The reaction was cooled to ambient temperature, and aqueous hydrochloric acid (1 N, 10 mL) was added. The reaction solution was extracted with ethyl acetate (3×20 mL). The combined organic layers were washed with brine (20 mL), dried over anhydrous magnesium sulfate and concentrated under vacuum. The residue was purified by silica gel chromatography using 0 to 50% hexane-ethyl acetate to furnish N-[4-(2,6-dimethylphenyl)-6-(2-fluorophenoxy)pyrimidin-2-yl]-1-methyl-1H-pyrazole-4-sulfonamide (250 mg, 92%) as a white solid. ESI-MS m/z calc. 453.12708, found 454.0 (M+1)⁺; Retention time: 5.0 minutes; ¹H NMR (500 MHz, DMSO-d₆) δ (ppm): 11.65 (s, 1H); 7.66 (s, 1H); 7.40 (m, 4H); 7.20 (m, 4H); 6.75 (s, 1H); 3.75 (s, 3H); 2.07 (s, 6H).

### Example 416: Preparation of Compound 1446

### Step 1: N-[4-(2-chloro-6-methyl-phenoxy)-6-(2,6-dimethylphenyl)pyrimidin-2-yl]-1 -methyl-pyrazole-4-sulfonamide

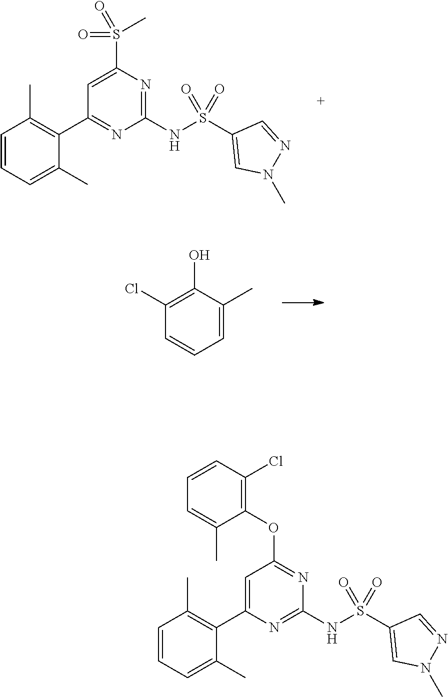

To a 10 mL vial equipped with a magnetic stir bar, N-[4-(2,6-dimethylphenyl)-6-methylsulfonyl-pyrimidin-2-yl]-1-methyl-pyrazole-4-sulfonamide (25.00 mg, 0.05931 mmol) (25.0 mg, 0.0593 mmol), N-methylpyrrolidinone (800 μL) and 2-chloro-6-methyl-phenol (32.79 mg, 0.23 mmol) were added, followed by potassium carbonate (31.79 mg, 0.23 mmol) (31.8 mg, 0.23 mmol). This mixture was stirred at 110° C. for 15 hours. The reaction mixture was then cooled to room temperature, quenched with 1 N HCl (1 mL), and extracted with ethyl acetate (3×1 mL). The combined organic extracts were washed with water (2×2 mL) and saturated aqueous sodium chloride solution (2 mL), then dried over sodium sulfate, filtered, and evaporated in vacuo. Purification by reverse phase HPLC (1-99% acetonitrile in water using HCl as a modifier) gave N-[4-(2-chloro-6-methyl-phenoxy)-6-(2,6-dimethylphenyl)pyrimidin-2-yl]-1-methyl-pyrazole-4-sulfonamide (5.2 mg, 58%). ESI-MS m/z calc. 483.1132, found 484.2 (M+1)⁺; Retention time: 1.82 minutes; LC method A.

### Example 417: Preparation of Compound 1447

### Step 1: N-[4-(2,6-Dimethylphenyl)-6-(2-isopropoxyphenoxy)pyrimidin-2-yl]-1 -methyl-pyrazole-4-sulfonamide

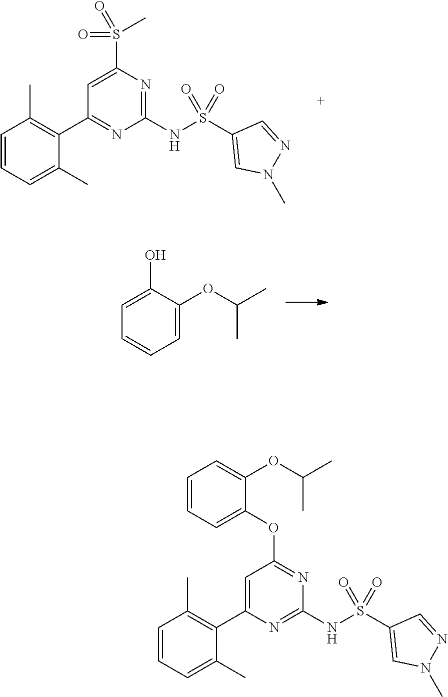 [SMILES: Cc1cccc(C)c1-c1cc(S(C)(=O)=O)nc(NS(=O)(=O)c2cnn(C)c2)n1 ; CC(C)Oc1ccccc1O ; Cc1cccc(C)c1-c1cc(Oc2ccccc2OC(C)C)nc(NS(=O)(=O)c2cnn(C)c2)n1]

To a 10 mL vial equipped with a magnetic stir bar, N-[4-(2,6-dimethylphenyl)-6-methylsulfonyl-pyrimidin-2-yl]-1-methyl-pyrazole-4-sulfonamide (15.11 mg, 0.03585 mmol) (15.1 mg, 0.03585 mmol), N-methylpyrrolidinone (500 μL) and 2-isopropoxyphenol (30.44 mg, 29.55 μL, 0.2 mmol) were added, followed by potassium carbonate (25.00 mg, 0.18087 mmol) (25.0 mg, 0.1809 mmol). This mixture was stirred at 110° C. for 18 hours. The reaction mixture was then cooled to room temperature, quenched with 1 N HCl (1 mL), and extracted with ethyl acetate (3×1 mL). The combined organic extracts were washed with water (2×2 mL) and saturated aqueous sodium chloride solution (2 mL), then dried over sodium sulfate, filtered, and evaporated in vacuo. Purification by reverse phase HPLC (1-99% acetonitrile in water using HCl as a modifier) gave the desired product N-[4-(2,6-dimethylphenyl)-6-(2-isopropoxyphenoxy)pyrimidin-2-yl]-1-methyl-pyrazole-4-sulfonamide (12.4 mg, 70%). ESI-MS m/z calc. 493.17838, found 494.41 (M+1)⁺; Retention time: 1.83 minutes; LC method A.

### Example 418: Preparation of Compound 1448

### Step 1: N-[4-(2,6-Dimethylphenoxy)-6-(2,6-dimethylphenyl)pyrimidin-2-yl]-1 -methyl-pyrazole-4-sulfonamide

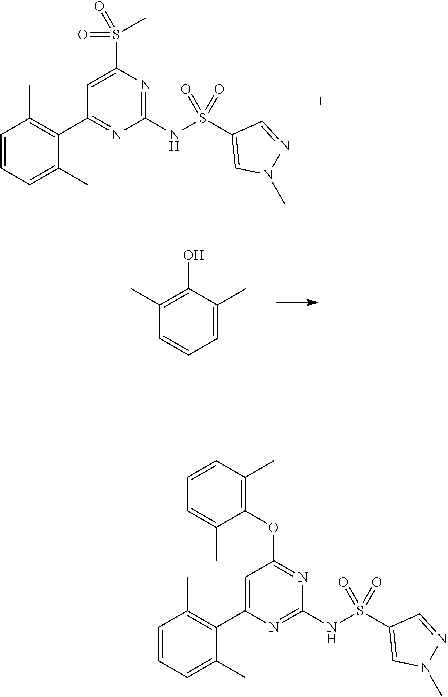

The compound was prepared in a manner analogous to that described above using commercially available 2,6-dimethylphenol gave N-[4-(2,6-dimethylphenoxy)-6-(2,6-dimethylphenyl)pyrimidin-2-yl]-1-methyl-pyrazole-4-sulfonamide (13.3 mg, 80%). ESI-MS m z calc. 463.16782, found 464.06 (M+1)⁺; Retention time: 1.83 minutes; LC method A.

### Example 419: Preparation of Compound 1449

### Step 1: N-[4-(2,6-Dimethylphenyl)-6-(2-methylphenoxy)pyrimidin-2-yl]-1-methyl-pyrazole-4-sulfonamide

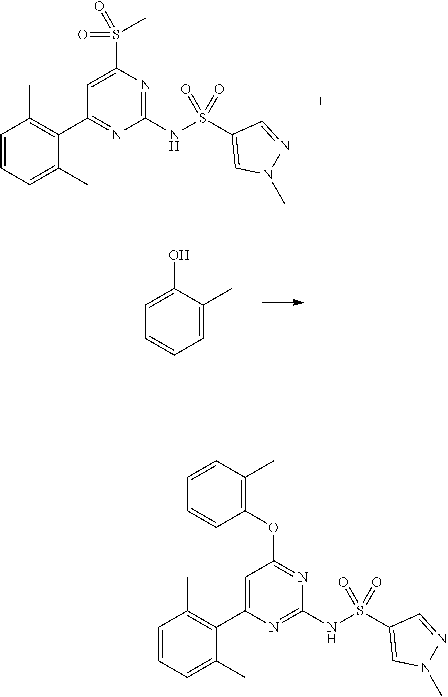

The compound was prepared in a manner analogous to that described above using commercially available o-cresol gave N-[4-(2,6-dimethylphenyl)-6-(2-methylphenoxy)pyrimidin-2-yl]-1-methyl-pyrazole-4-sulfonamide (12.4 mg, 74%). ESI-MS m z calc. 449.15216, found 450.05 (M+1)⁺; Retention time: 1.72 minutes; LC method A.

### Example 420: Preparation of Compound 1450

### Step 1: N-[4-(2,6-Difluorophenoxy)-6-(2,6-dimethylphenyl)pyrimidin-2-yl]-1 -methyl-pyrazole-4-sulfonamide

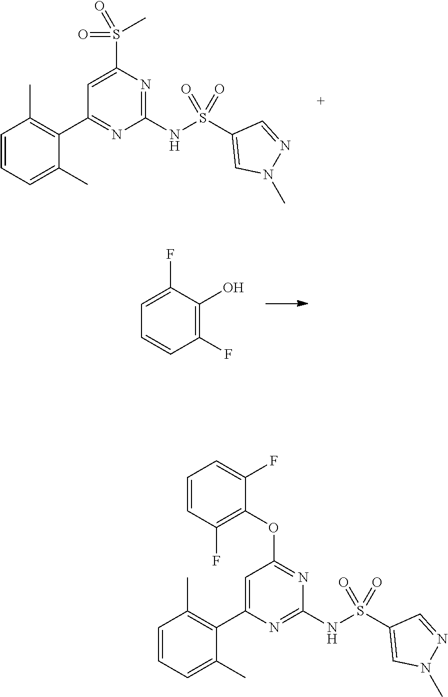 [SMILES: Cc1cccc(C)c1-c1cc(S(C)(=O)=O)nc(NS(=O)(=O)c2cnn(C)c2)n1 ; Oc1c(F)cccc1F ; Cc1cccc(C)c1-c1cc(Oc2c(F)cccc2F)nc(NS(=O)(=O)c2cnn(C)c2)n1]

The compound was prepared in a manner analogous to that described above using commercially available 2,6-difluorophenol to give N-[4-(2,6-difluorophenoxy)-6-(2,6-dimethylphenyl)pyrimidin-2-yl]-1-methyl-pyrazole-4-sulfonamide (13.2 mg, 78%). ESI-MS m z calc. 471.11768, found 472.15 (M+1)⁺; Retention time: 1.77 minutes; LC method A.

### Example 421: Preparation of Compound 1451

### Step 1: N-[4-(2-chloro-6-fluoro-phenoxy)-6-(2,6-dimethylphenyl)pyrimidin-2-yl]-1 -methyl-pyrazole-4-sulfonamide

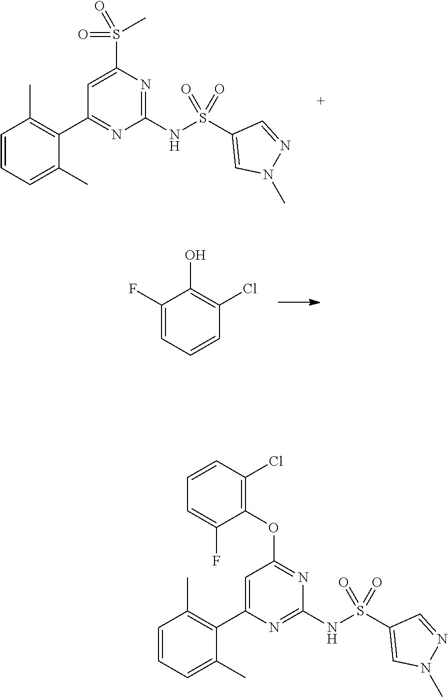 [SMILES: Cc1cccc(C)c1-c1cc(S(C)(=O)=O)nc(NS(=O)(=O)c2cnn(C)c2)n1 ; Oc1c(F)cccc1Cl ; Cc1cccc(C)c1-c1cc(Oc2c(F)cccc2Cl)nc(NS(=O)(=O)c2cnn(C)c2)n1]

The compound was prepared in a manner analogous to that described above using commercially available 2-chloro-6-fluoro-phenol to give N-[4-(2-chloro-6-fluoro-phenoxy)-6-(2,6-dimethylphenyl)pyrimidin-2-yl]-1-methyl-pyrazole-4-sulfonamide (14.2 mg, 81%). ESI-MS m/z calc. 487.0881, found 488.32 (M+1)⁺; Retention time: 1.82 minutes; LC method A.

### Example 422: Preparation of Compound 1452

### Step 1: N-[4-(4-Cyano-2,6-dimethyl-phenoxy)-6-(2,6-dimethylphenyl)pyrimidin-2-yl]-1-methyl-pyrazole-4-sulfonamide

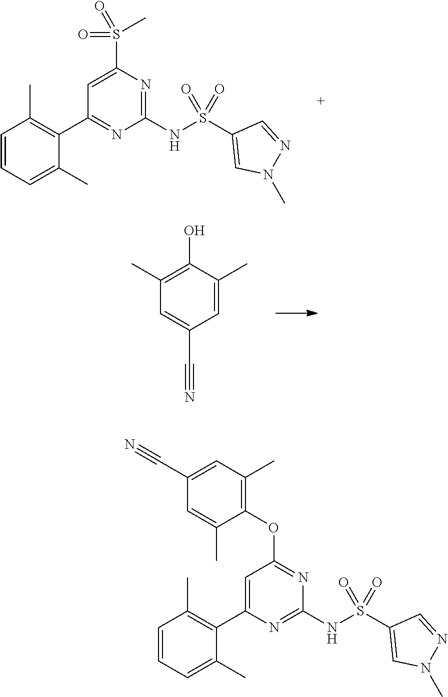

The compound was prepared in a manner analogous to that described above using commercially available 4-hydroxy-3,5-dimethyl-benzonitrile to give N-[4-(4-cyano-2,6-dimethyl-phenoxy)-6-(2,6-dimethylphenyl)pyrimidin-2-yl]-1-methyl-pyrazole-4-sulfonamide (9.1 mg, 51%). ESI-MS m/z calc. 488.16306, found 489.2 (M+1)⁺; Retention time: 1.74 minutes; LC method A.

### Example 423: Preparation of Compound 1453

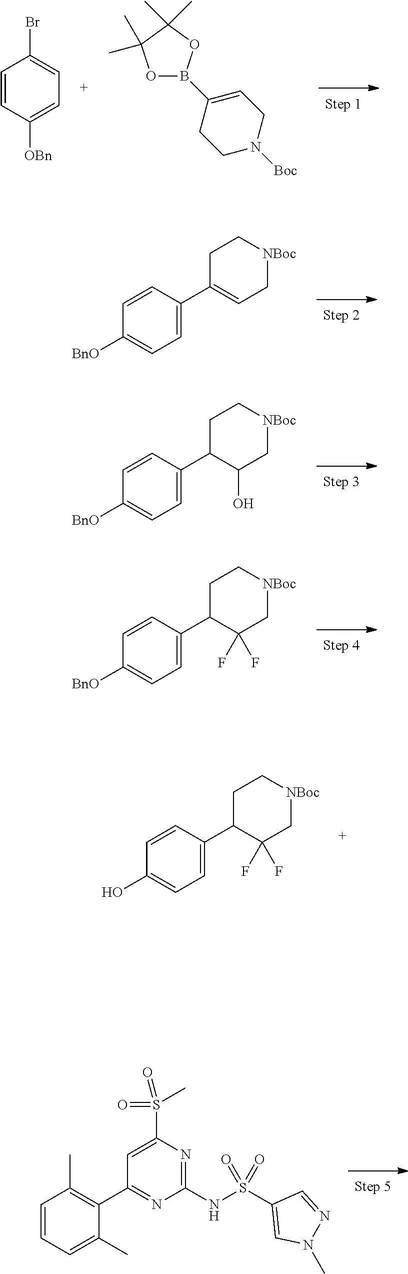

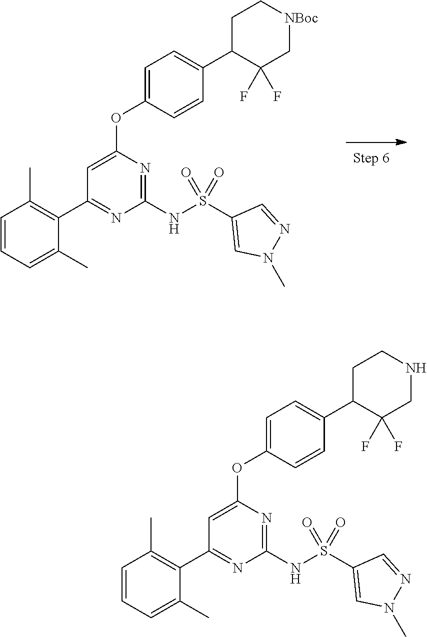 [SMILES: Cc1cccc(C)c1-c1cc(Oc2ccc(C3CCN(C(=O)OC(C)(C)C)CC3(F)F)cc2)nc(NS(=O)(=O)c2cnn(C)c2)n1 ; Cc1cccc(C)c1-c1cc(Oc2ccc(C3CCNCC3(F)F)cc2)nc(NS(=O)(=O)c2cnn(C)c2)n1]

### Step 1: tert-Butyl 4-(4-benzyloxyphenyl)-3,6-dihydro-2H-pyridine-1-carboxylate

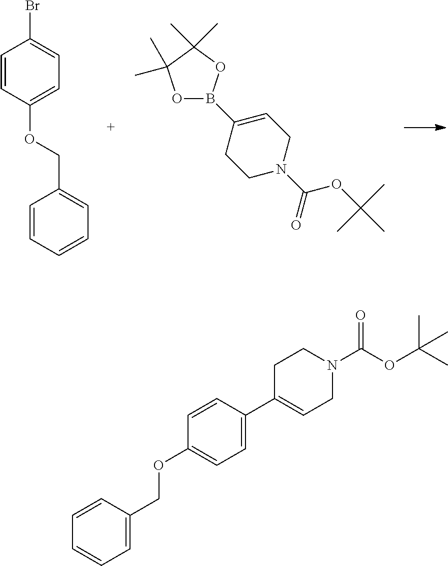 [SMILES: Brc1ccc(OCc2ccccc2)cc1 ; CC(C)(C)OC(=O)N1CC=C(B2OC(C)(C)C(C)(C)O2)CC1 ; CC(C)(C)OC(=O)N1CC=C(c2ccc(OCc3ccccc3)cc2)CC1]

To a solution of 1-benzyloxy-4-bromo-benzene (2.8 g, 10.64 mmol), tert-butyl 4-(4,4,5,5-tetramethyl-1,3,2-dioxaborolan-2-yl)-3,6-dihydro-2H-pyridine-1-carboxylate (3.38 g, 10.93 mmol) and bis(triphenylphosphine)palladium(II) dichloride (383 mg, 0.546 mmol) in DME (50 mL) and water (20 mL) was added sodium carbonate (3.77 g, 35.57 mmol) and the reaction mixture was stirred at 80° C. for 6 hours. The reaction mixture was filtered through Celite, poured into water and extracted with EtOAc (3×). The organics were combined, washed with water then brine, dried over sodium sulfate, and evaporated to dryness. Purification by column chromatography (120 g silica; 0-20% EtOAc in hexanes) gave tert-butyl 4-(4-benzyloxyphenyl)-3,6-dihydro-2H-pyridine-1-carboxylate (2.48 g, 62%) as a white solid. ESI-MS m/z calc. 365.1991, found 366.3 (M+1)⁺; Retention time: 0.82 minutes; LC method D.

### Step 2: tert-Butyl 4-(4-benzyloxyphenyl)-3-hydroxy-piperidine-1-carboxylate

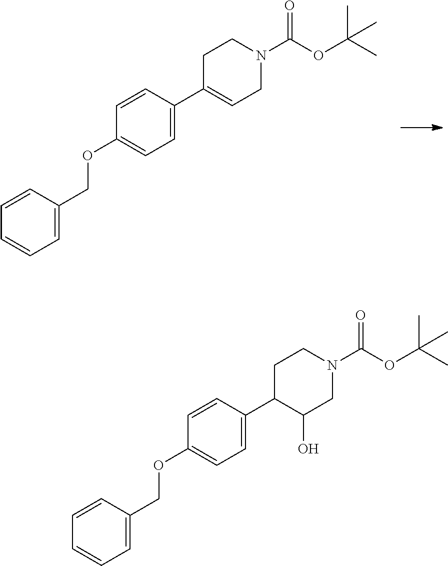 [SMILES: CC(C)(C)OC(=O)N1CC=C(c2ccc(OCc3ccccc3)cc2)CC1 ; CC(C)(C)OC(=O)N1CCC(c2ccc(OCc3ccccc3)cc2)C(O)C1]

To a solution of borane dimethylsulfide (approximately 1.658 mL of 2 M, 3.317 mmol) in THE (10.10 mL) at 0° C. was added a solution of tert-butyl 4-(4-benzyloxyphenyl)-3,6-dihydro-2H-pyridine-1-carboxylate (1.01 g, 2.764 mmol) in THE (5 mL) dropwise. The cooling bath was removed and stirred at room temperature for 2 hours. The reaction mixture was cooled to 0° C. and NaOH (approximately 1.013 mL of 3 M, 3.040 mmol), H₂O₂ (approximately 1.034 mL of 20% w/v, 6.081 mmol) and EtOH (1 mL) were added sequentially. The cooling bath was removed, and the reaction mixture stirred at 60° C. for 4 hours. The reaction mixture was poured into water and extracted with EtOAc (3×). The organic were combined, washed with water and brine, dried over sodium sulfate, filtered through a short plug of silica and evaporated to dryness to give tert-butyl 4-(4-benzyloxyphenyl)-3-hydroxy-piperidine-1-carboxylate (990 mg, 93%) as a white solid. ESI-MS m/z calc. 383.20966, found 384.3 (M+1)⁺; Retention time: 0.71 minutes; LC method D.

### Step 3: tert-Butyl 4-(4-benzyloxyphenyl)-3,3-difluoro-piperidine-1-carboxylate

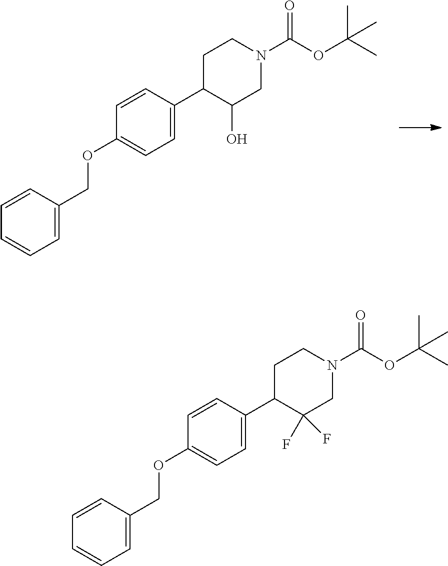 [SMILES: CC(C)(C)OC(=O)N1CCC(c2ccc(OCc3ccccc3)cc2)C(O)C1 ; CC(C)(C)OC(=O)N1CCC(c2ccc(OCc3ccccc3)cc2)C(F)(F)C1]

To a solution of tert-butyl 4-(4-benzyloxyphenyl)-3-hydroxy-piperidine-1-carboxylate (250 mg, 0.6519 mmol) in DCM (5 mL) was added Dess-Martin periodinane (0.85 g, 2.004 mmol) and the reaction mixture was stirred at rt for 2 hours. The reaction mixture was filtered through Celite, and the pad was washed with EtOAc. The filtrate was evaporated to dryness. Purification by column chromatography (12 g silica; 0-50% EtOAc in hexanes) gave tert-butyl 4-(4-benzyloxyphenyl)-3-oxo-piperidine-1-carboxylate. A solution of the ketone in DCM (4 mL) was cooled to −78° C. and bis(2-methoxyethyl)aminosulfur trifluoride (Deoxo-Fluor®, 480 μL, 2.60 mmol) was added dropwise and stirred at this temp or 1 hour. The cooling bath was removed, and the reaction mixture was stirred at room temperature for 16 hours. The reaction mixture was slowly and carefully quenched with ice, then slowly poured into 1 M sodium carbonate and extracted with DCM (3×). The organics were combined, washed with water, brine, dried over sodium sulfate and evaporated to dryness. Purification by column chromatography (12 g silica; 0-50% EtOAc in hexanes) gave tert-butyl 4-(4-benzyloxyphenyl)-3,3-difluoro-piperidine-1-carboxylate (40 mg, 15%) as a clear oil. ESI-MS m/z calc. 403.1959, found 348.2 (M+1)⁺; Retention time: 0.8 minutes (LC method D).

### Step 4: tert-Butyl 3,3-difluoro-4-(4-hydroxyphenyl)piperidine-1-carboxylate

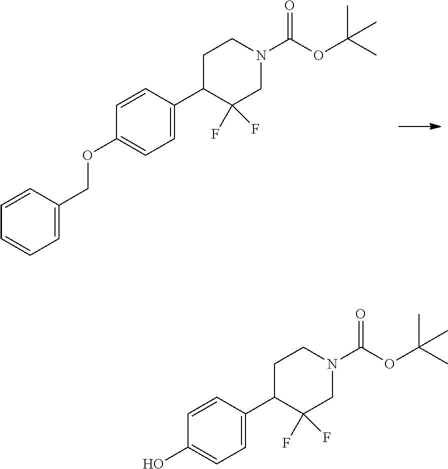 [SMILES: CC(C)(C)OC(=O)N1CCC(c2ccc(OCc3ccccc3)cc2)C(F)(F)C1 ; CC(C)(C)OC(=O)N1CCC(c2ccc(O)cc2)C(F)(F)C1]

To a suspension of tert-butyl 4-(4-benzyloxyphenyl)-3,3-difluoro-piperidine-1-carboxylate (40 mg, 0.09914 mmol) and Pd/C (approximately 105 mg of 10% w/w, 0.0991 mmol) in MeOH (0.5 mL) was stirred under a balloon of hydrogen for 2 hours. The reaction mixture was filtered through Celite and evaporated to dryness to give tert-butyl 3,3-difluoro-4-(4-hydroxyphenyl)piperidine-1-carboxylate (30 mg, 97%). Used without further purification. ESI-MS m/z calc. 313.14896, found 258.1 (M+1)⁺; Retention time: 0.6 minutes (LC method D).

### Step 5: tert-Butyl 4-[4-[6-(2,6-dimethylphenyl)-2-[(1-methylpyrazol-4-yl)sulfonylamino]pyrimidin-4-yl]oxyphenyl]-3,3-difluoro-piperidine-1-carboxylate

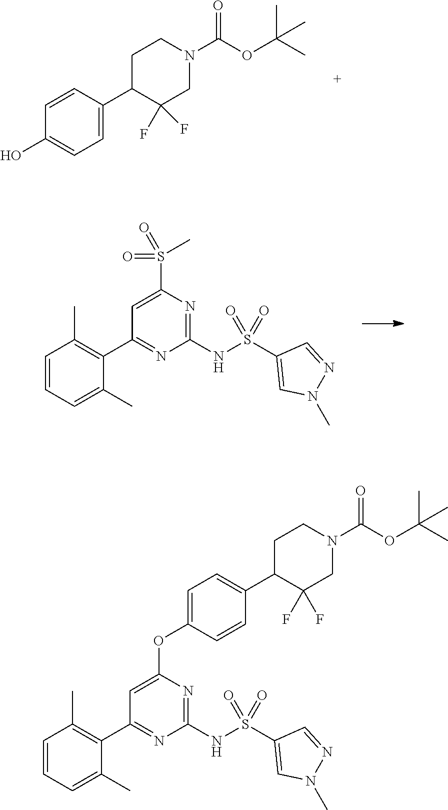 [SMILES: CC(C)(C)OC(=O)N1CCC(c2ccc(O)cc2)C(F)(F)C1 ; Cc1cccc(C)c1-c1cc(S(C)(=O)=O)nc(NS(=O)(=O)c2cnn(C)c2)n1 ; Cc1cccc(C)c1-c1cc(Oc2ccc(C3CCN(C(=O)OC(C)(C)C)CC3(F)F)cc2)nc(NS(=O)(=O)c2cnn(C)c2)n1]

To a solution of N-[4-(2,6-dimethylphenyl)-6-methylsulfonyl-pyrimidin-2-yl]-1 -methyl-pyrazole-4-sulfonamide (31 mg, 0.07355 mmol) in NMP (0.5 mL) was added Cs₂CO₃ (75 mg, 0.2302 mmol) and tert-butyl 3,3-difluoro-4-(4-hydroxyphenyl)piperidine-1-carboxylate (20 mg, 0.06383 mmol) and the reaction mixture stirred at 100° C. for 10 minutes, then for 60° C. for 16 hours. The reaction mixture was diluted with MeOH, filtered and purification by HPLC (1-99% ACN in water (HCl modifier)) gave tert-butyl 4-[4-[6-(2,6-dimethylphenyl)-2-[(1-methylpyrazol-4-yl)sulfonylamino]pyrimidin-4-yl]oxyphenyl]-3,3-difluoro-piperidine-1-carboxylate (25.0 mg, 60%). ESI-MS m/z calc. 654.2436, found 551.9 (M+1)⁺; Retention time: 0.99 minutes; (LC method A).

### Step 6: N-[4-[4-(3,3-Difluoro-4-piperidyl)phenoxy]-6-(2,6-dimethylphenyl)pyrimidin-2-yl]-1-methyl-pyrazole-4-sulfonamide

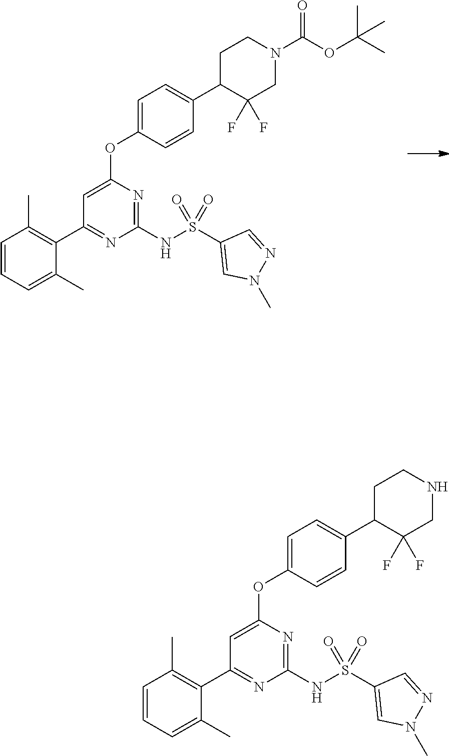 [SMILES: Cc1cccc(C)c1-c1cc(Oc2ccc(C3CCN(C(=O)OC(C)(C)C)CC3(F)F)cc2)nc(NS(=O)(=O)c2cnn(C)c2)n1 ; Cc1cccc(C)c1-c1cc(Oc2ccc(C3CCNCC3(F)F)cc2)nc(NS(=O)(=O)c2cnn(C)c2)n1]

To a solution of tert-butyl 4-[4-[6-(2,6-dimethylphenyl)-2-[(1-methylpyrazol-4-yl)sulfonylamino]pyrimidin-4-yl]oxyphenyl]-3,3-difluoro-piperidine-1-carboxylate (25 mg, 0.03818 mmol) in DCM (0.5 mL) was added HCl (4 M in dioxane) (250 μL of 4 M, 1.000 mmol) and the reaction mixture was stirred at room temperature for 1 hour. The reaction mixture was diluted with MeOH and purification by HPLC (1-99% ACN in water (HCl modifier)) gave N-[4-[4-(3,3-difluoro-4-piperidyl)phenoxy]-6-(2,6-dimethylphenyl)pyrimidin-2-yl]-1-methyl-pyrazole-4-sulfonamide (hydrochloride salt) (15 mg, 67%) as a white solid. ESI-MS m/z calc. 554.19116, found 555.4 (M+1)⁺; Retention time: 1.13 minutes; LC method A.

### Example 424: Preparation of Compound 1454

### Step 1: N-[4-[4-(Azepan-1-yl)phenoxy]-6-(2,6-dimethylphenyl)pyrimidin-2-yl]-1 -methyl-pyrazole-4-sulfonamide

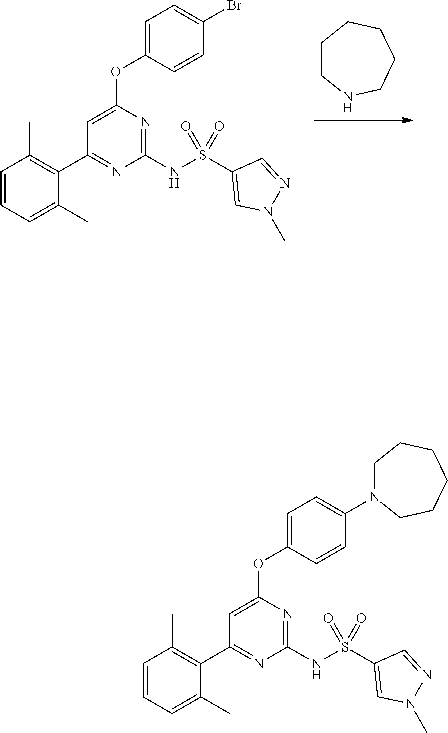 [SMILES: Cc1cccc(C)c1-c1cc(Oc2ccc(Br)cc2)nc(NS(=O)(=O)c2cnn(C)c2)n1 ; C1CCCNCC1 ; Cc1cccc(C)c1-c1cc(Oc2ccc(N3CCCCCC3)cc2)nc(NS(=O)(=O)c2cnn(C)c2)n1]

In a glass vial were combined N-[4-(4-bromophenoxy)-6-(2,6-dimethylphenyl)pyrimidin-2-yl]-1-methyl-pyrazole-4-sulfonamide (20 mg, 0.03888 mmol), [2-(2-aminoethyl)phenyl]-chloro-palladium;ditert-butyl-[2-(2,4,6-triisopropylphenyl)phenyl]phosphane (XPhos Pd G1)(approximately 10.68 mg, 0.01555 mmol), azepane (approximately 11.56 mg, 13.14 μL, 0.1166 mmol), sodium tert-butoxide (approximately 22.42 mg, 0.2333 mmol), and dioxane (0.8 mL) and the mixture was sparged with nitrogen under sonication for 5 minutes. It was then stirred at room temperature for 15 minutes. AcOH (approximately 2.335 mg, 2.211 μL, 0.03888 mmol) was added to quench the reaction. The solution was filtered, and the resulting residues dissolved in 0.8 mL MeOH and purified by reverse-phase preparative HPLC using a 15 min gradient of 20% MeCN in water to 80% MeCN to afford N-[4-[4-(azepan-1-yl)phenoxy]-6-(2,6-dimethylphenyl)pyrimidin-2-yl]-1 -methyl-pyrazole-4-sulfonamide (4.2 mg, 20%). ESI-MS m/z calc. 532.22565, found 533.6 (M+1)⁺; Retention time: 1.84 minutes; LC method A.

### Example 425: Preparation of Compound 1455 and Compound 1456

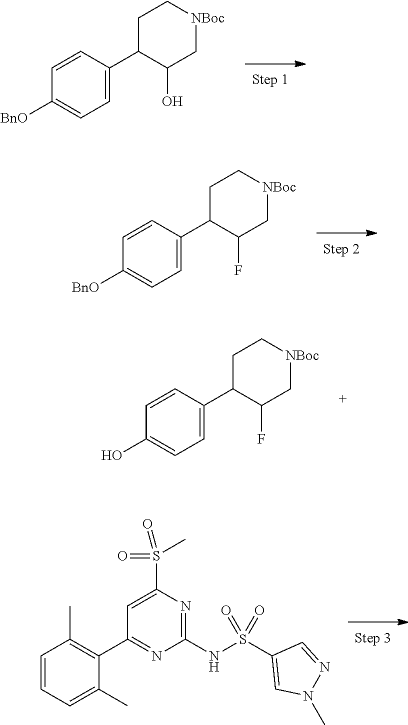 [SMILES: CC(C)(C)OC(=O)N1CCC(c2ccc(OCc3ccccc3)cc2)C(O)C1 ; CC(C)(C)OC(=O)N1CCC(c2ccc(OCc3ccccc3)cc2)C(F)C1 ; CC(C)(C)OC(=O)N1CCC(c2ccc(O)cc2)C(F)C1 ; Cc1cccc(C)c1-c1cc(S(C)(=O)=O)nc(NS(=O)(=O)c2cnn(C)c2)n1]

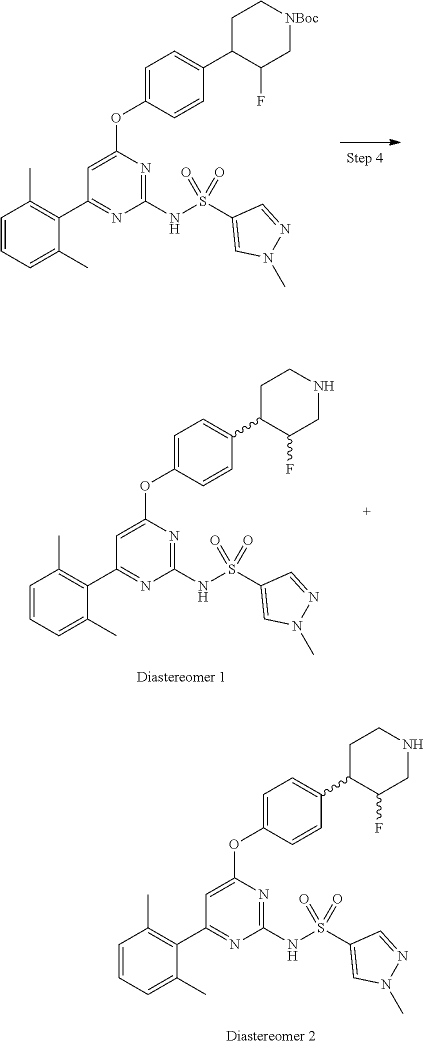 [SMILES: Cc1cccc(C)c1-c1cc(Oc2ccc(C3CCN(C(=O)OC(C)(C)C)CC3F)cc2)nc(NS(=O)(=O)c2cnn(C)c2)n1 ; Cc1cccc(C)c1-c1cc(Oc2ccc(C3CCNCC3F)cc2)nc(NS(=O)(=O)c2cnn(C)c2)n1 ; Cc1cccc(C)c1-c1cc(Oc2ccc(C3CCNCC3F)cc2)nc(NS(=O)(=O)c2cnn(C)c2)n1]

### Step 1: tert-Butyl 4-(4-benzyloxyphenyl)-3-fluoro-piperidine-1-carboxylate

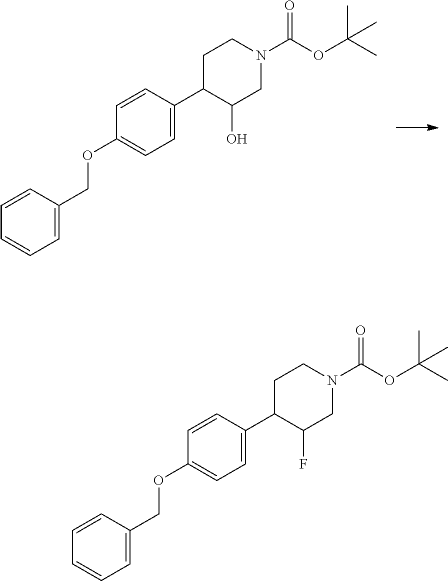 [SMILES: CC(C)(C)OC(=O)N1CCC(c2ccc(OCc3ccccc3)cc2)C(O)C1 ; CC(C)(C)OC(=O)N1CCC(c2ccc(OCc3ccccc3)cc2)C(F)C1]

To a solution of tert-butyl 4-(4-benzyloxyphenyl)-3-hydroxy-piperidine-1-carboxylate (335 mg, 0.8736 mmol) in DCM (5 mL) at −78° C. was added bis(2-methoxyethyl)aminosulfur trifluoride (Deoxo-Fluor®, 33 mg, 0.1492 mmol) dropwise and the reaction mixture was stirred at this temperature for 1 hour. The cooling bath was removed, and the reaction mixture was stirred at room temperature for 16 hours. The reaction mixture was poured into water and extracted with DCM (3×). The organics were dried over sodium sulfate, filtered through a short plug of silica (eluted with 3 column volumes of EtOAc) and evaporated to dryness to give tert-butyl 4-(4-benzyloxyphenyl)-3-fluoro-piperidine-1-carboxylate (302 mg, 90%) as a pale-yellow oil. ESI-MS m/z calc. 385.20532, found 386.3 (M+1)⁺; Retention time: 0.77 minutes; LC method D.

### Step 2: tert-Butyl 3-fluoro-4-(4-hydroxyphenyl)piperidine-1-carboxylate

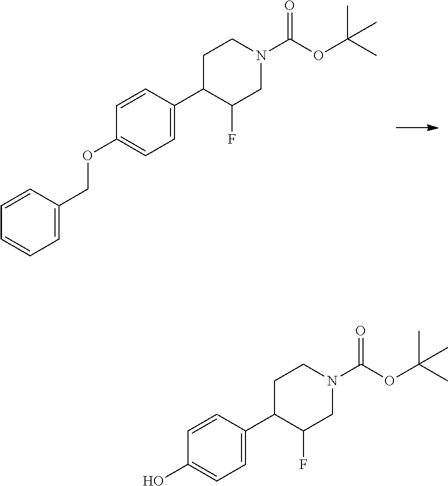 [SMILES: CC(C)(C)OC(=O)N1CCC(c2ccc(OCc3ccccc3)cc2)C(F)C1 ; CC(C)(C)OC(=O)N1CCC(c2ccc(O)cc2)C(F)C1]

A suspension of tert-butyl 4-(4-benzyloxyphenyl)-3-fluoro-piperidine-1-carboxylate (302 mg, 0.7835 mmol) and Pd/C (30 mg of 10% w/w, 0.02819 mmol) in EtOH (3 mL)/EtOAc (1 mL) was stirred under a balloon of hydrogen for 3 hours. At this point the reaction mixture was heated to 50° C. and stirred at this temp for 16 hours. The reaction mixture was filtered through Celite and evaporated to dryness. More Pd/C (30 mg of 10% w/w, 0.02819 mmol) was added and the residue was taken up in EtOH (3 mL) and EtOAc (1 mL) and stirred at 50° C. under a balloon of hydrogen for 6 hours. The reaction mixture was filtered through Celite and evaporated to dryness. More Pd/C (30 mg of 10% w/w, 0.02819 mmol) was added and the residue was taken up in EtOH (3 mL) and EtOAc (1 mL) and stirred at 50° C. under a balloon of hydrogen for 16 hours. The reaction mixture was filtered through Celite and evaporated to dryness and used without further purification. ESI-MS m/z calc. 295.1584, found 296.2 (M+1)⁺; Retention time: 0.57 minutes; LC method D.

### Step 3: tert-Butyl 4-[4-[6-(2,6-dimethylphenyl)-2-[(1-methylpyrazol-4-yl)sulfonylamino]pyrimidin-4-yl]oxyphenyl]-3-fluoro-piperidine-1-carboxylate

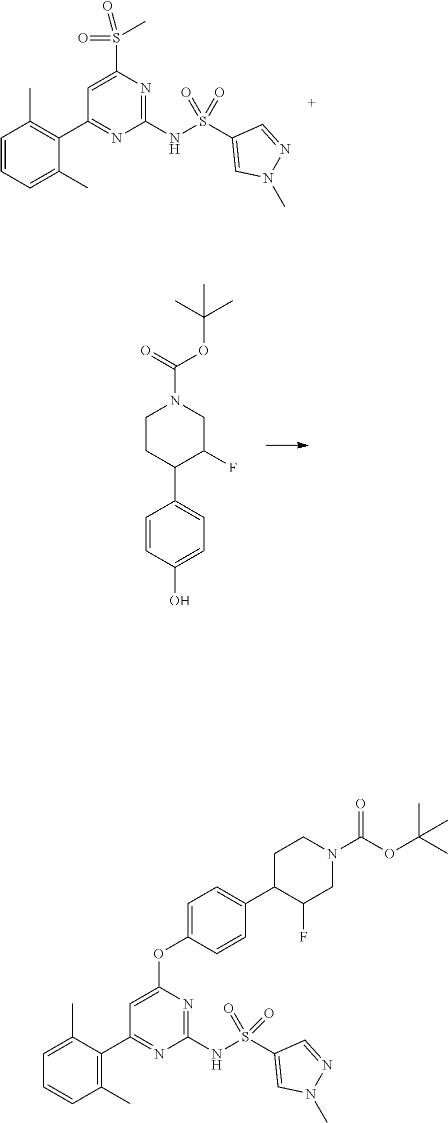 [SMILES: Cc1cccc(C)c1-c1cc(S(C)(=O)=O)nc(NS(=O)(=O)c2cnn(C)c2)n1 ; CC(C)(C)OC(=O)N1CCC(c2ccc(O)cc2)C(F)C1 ; Cc1cccc(C)c1-c1cc(Oc2ccc(C3CCN(C(=O)OC(C)(C)C)CC3F)cc2)nc(NS(=O)(=O)c2cnn(C)c2)n1]

To a solution of N-[4-(2,6-dimethylphenyl)-6-methylsulfonyl-pyrimidin-2-yl]-1 -methyl-pyrazole-4-sulfonamide (42 mg, 0.09965 mmol) and tert-butyl 3-fluoro-4-(4-hydroxyphenyl)piperidine-1-carboxylate (45 mg, 0.1524 mmol) in NMP (0.5 mL) was added Cs₂CO₃ (152 mg, 0.4665 mmol) and the reaction mixture stirred at 80° C. for 16 hours. The reaction mixture was diluted with water, the pH brought to 4 with 1 N HCl and the product was extracted with EtOAc (3×). The organics were combined, washed with water, brine, dried over sodium sulfate, and evaporated to dryness. Purification by column chromatography (12 g silica; 0-50% EtOAc in hexanes) gave tert-butyl 4-[4-[6-(2,6-dimethylphenyl)-2-[(1-methylpyrazol-4-yl)sulfonylamino]pyrimidin-4-yl]oxyphenyl]-3-fluoro-piperidine-1-carboxylate (36.8 mg, 58%) as a glass. ESI-MS m/z calc. 636.253, found 637.4 (M+1)⁺; Retention time: 0.74 minutes; LC method D.

### Step 4: N-[4-(2,6-Dimethylphenyl)-6-[4-(3-fluoro-4-piperidyl)phenoxy]pyrimidin-2-yl]-1-methyl-pyrazole-4-sulfonamide, diasteromer 1, Compound 1455 and N-[4-(2,6-dimethylphenyl)-6-[4-(3-fluoro-4-piperidyl)phenoxy]pyrimidin-2-yl]-1-methyl-pyrazole-4-sulfonamide, diastereomer 2, Compound 1456

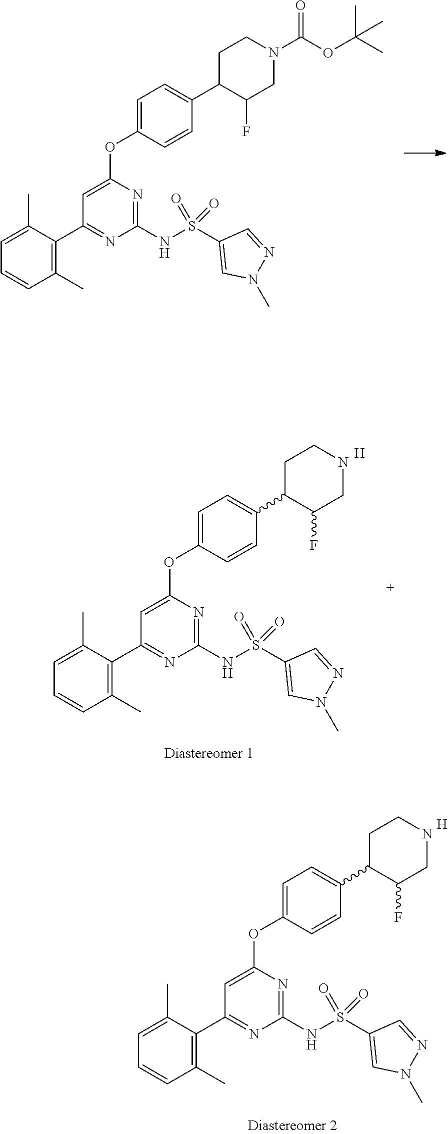

To a solution of tert-butyl 4-[4-[6-(2,6-dimethylphenyl)-2-[(1-methylpyrazol-4-yl)sulfonylamino]pyrimidin-4-yl]oxyphenyl]-3-fluoro-piperidine-1-carboxylate (36 mg, 0.05654 mmol) in DCM (0.5 mL) was added HCl (290 μL of 4 M, 1.16 mmol) dropwise and the reaction mixture was stirred at room temperature for 20 minutes. The reaction mixture was diluted with MeOH, filtered. Purification by HPLC (1-99% ACN in water (HCl modifier)) [30 minute run]gave two isomers: First to elute diastereomer 1, N-[4-(2,6-dimethylphenyl)-6-[4-(3-fluoro-4-piperidyl)phenoxy]pyrimidin-2-yl]-1-methyl-pyrazole-4-sulfonamide (hydrochloride salt) (4.7 mg, 29%), ESI-MS m/z calc. 536.20056, found 537.3 (M+1)⁺; Retention time: 1.15 minutes (LC method A); and second to elute diastereomer 2, N-[4-(2,6-dimethylphenyl)-6-[4-(3-fluoro-4-piperidyl)phenoxy]pyrimidin-2-yl]-1-methyl-pyrazole-4-sulfonamide (hydrochloride salt) (1.4 mg, 9%) ESI-MS m/z calc. 536.20056, found 537.3 (M+1)⁺; Retention time: 1.17 minutes (LC method A).

### Example 426: Preparation of Compound 1457

### Step 1: N-[4-(2,6-Dimethylphenyl)-6-[4-(1,4-oxazepan-4-yl)phenoxy]pyrimidin-2-yl]-1-methyl-pyrazole-4-sulfonamide

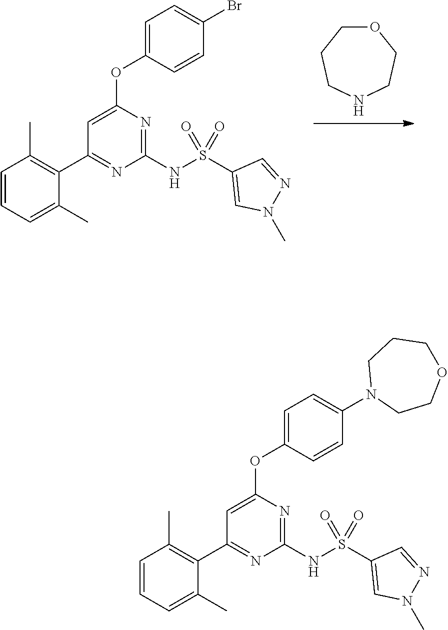 [SMILES: Cc1cccc(C)c1-c1cc(Oc2ccc(Br)cc2)nc(NS(=O)(=O)c2cnn(C)c2)n1 ; C1CNCCOC1 ; Cc1cccc(C)c1-c1cc(Oc2ccc(N3CCCOCC3)cc2)nc(NS(=O)(=O)c2cnn(C)c2)n1]

The compound was prepared in a manner analogous to that described above using commercially available 1,4-oxazepane to give N-[4-(2,6-dimethylphenyl)-6-[4-(1,4-oxazepan-4-yl)phenoxy]pyrimidin-2-yl]-1-methyl-pyrazole-4-sulfonamide (7 mg, 32%). ESI-MS m/z calc. 534.2049, found 535.42 (M+1)⁺; Retention time: 1.17 minutes; LC method A.

### Example 427: Preparation of Compound 1458

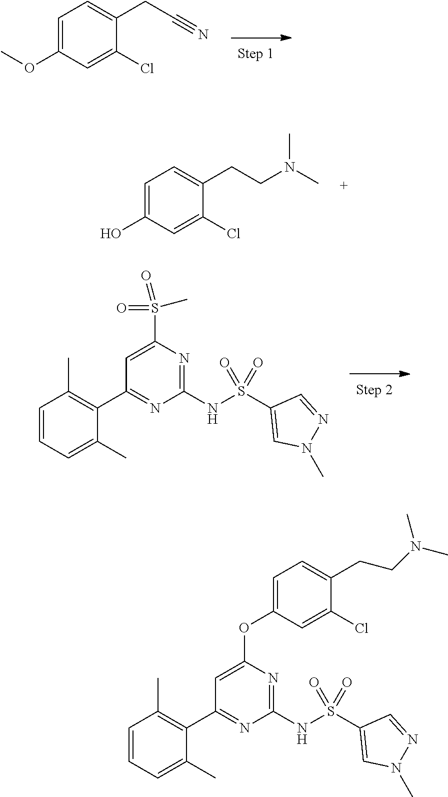 [SMILES: COc1ccc(CC#N)c(Cl)c1 ; CN(C)CCc1ccc(O)cc1Cl ; Cc1cccc(C)c1-c1cc(S(C)(=O)=O)nc(NS(=O)(=O)c2cnn(C)c2)n1 ; Cc1cccc(C)c1-c1cc(Oc2ccc(CCN(C)C)c(Cl)c2)nc(NS(=O)(=O)c2cnn(C)c2)n1]

### Step 1: 3-chloro-4-[2-(dimethylamino)ethyl]phenol

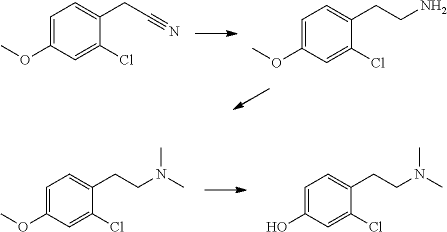

To a solution of 2-(2-chloro-4-methoxy-phenyl)acetonitrile (300 mg, 1.65 mmol) in MeOH (7 mL) was added sodium borohydride (652 mg, 17.2 mmol) and nickel chloride (214 mg, 1.65 mmol) at 0° C. under N2 atmosphere, slowly and in small portions. The reaction mixture was allowed to warm to room temperature and stirred at room temperature for 1.5 hours. The reaction mixture was diluted with EtOAc and then washed with brine. The organic layer was dried over anhydrous sodium sulfate, filtered through Celite, and the filtrate was concentrated to give 2-(2-chloro-4-methoxy-phenyl)ethanamine (306 mg, 100%). ESI-MS m z calc. 185.06075, found 186.1 (M+1)⁺; Retention time: 0.35 minutes; LC method D.

The residue was taken up in formic acid (200 μL, 5.30 mmol) and formaldehyde (200 μL, 7.26 mmol) and stirred at 90° C. for 30 minutes and then cooled to room temperature. The reaction mixture was diluted with water (10 mL) and washed with ethyl acetate (15 mL). The two layers was separated, and the aqueous layer was carefully quenched with 1 M NaOH until the pH was basic (˜10-11). The aqueous layer was extracted with ethyl acetate (2×15 mL). The organic layer was dried over anhydrous sodium sulfate, filtered, and concentrated in vacuo to give 2-(2-chloro-4-methoxy-phenyl)-N,N-dimethyl-ethanamine (353 mg, 100%). ESI-MS m z calc. 213.09204, found 214.23 (M+1)⁺; Retention time: 0.34 minutes; LC method D.

The residue was taken up in aqueous 48 wt % HBr (300 μL, 2.65 mmol) and stirred at 110° C. for 1 hour and then concentrated in vacuo. The resulting residue was triturated in diethyl ether and then filtered to give 3-chloro-4-[2-(dimethylamino)ethyl]phenol (hydrobromic salt) (42.9 mg, 9%). ESI-MS m/z calc. 199.07639, found 200.22 (M+1)⁺; Retention time: 0.53 minutes; LC method A.

### Step 2: N-[4-[3-chloro-4-[2-(dimethylamino)ethyl]phenoxy]-6-(2,6-dimethylphenyl)pyrimidin-2-yl]-1-methyl-pyrazole-4-sulfonamide

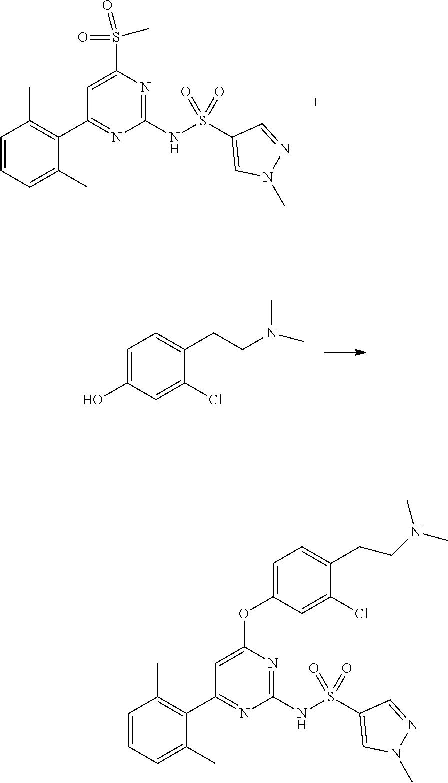 [SMILES: Cc1cccc(C)c1-c1cc(S(C)(=O)=O)nc(NS(=O)(=O)c2cnn(C)c2)n1 ; CN(C)CCc1ccc(O)cc1Cl ; Cc1cccc(C)c1-c1cc(Oc2ccc(CCN(C)C)c(Cl)c2)nc(NS(=O)(=O)c2cnn(C)c2)n1]

An NMP (0.5 mL) mixture of N-[4-(2,6-dimethylphenyl)-6-methylsulfonyl-pyrimidin-2-yl]-1-methyl-pyrazole-4-sulfonamide (12.7 mg, 0.0289 mmol), Cs₂CO₃ (50.8 mg, 0.156 mmol), and 3-chloro-4-[2-(dimethylamino)ethyl]phenol (hydrobromic salt) (15.8 mg, 0.0563 mmol) was heated to 110° C. for 1 hour and then cooled to room temperature. The solution was filtered and the resulting residue dissolved in 0.8 mL MeOH, and injected onto a reverse-phase preparative HPLC for chromatography using a 15 minutes gradient of 20% MeCN in water to 80% MeCN (HCl modifier) to give N-[4-[3-chloro-4-[2-(dimethylamino)ethyl]phenoxy]-6-(2,6-dimethylphenyl)pyrimidin-2-yl]-1-methyl-pyrazole-4-sulfonamide (hydrochloride salt) (3.6 mg, 22%). ¹H NMR (400 MHz, DMSO-d₆) δ 10.32 (s, 1H), 7.84-7.72 (m, 1H), 7.66-7.52 (m, 2H), 7.36 (dd, J=8.4, 2.5 Hz, 1H), 7.30-7.22 (m, 1H), 7.21-7.10 (m, 3H), 6.69 (s, 1H), 3.76 (s, 3H), 3.36-3.23 (m, 2H), 3.23-3.12 (m, 2H), 2.86 (d, J=4.9 Hz, 6H), 2.05 (s, 6H). ESI-MS m/z calc. 540.171, found 541.5 (M+1)⁺; Retention time: 1.25 minutes; LC method A.

### Example 428: Preparation of Compound 1459

### Step 1: N-[4-(2,6-Dimethylphenyl)-6-[4-(3-fluoro-1-methyl-4-piperidyl)phenoxy]pyrimidin-2-yl]-1-methyl-pyrazole-4-sulfonamide

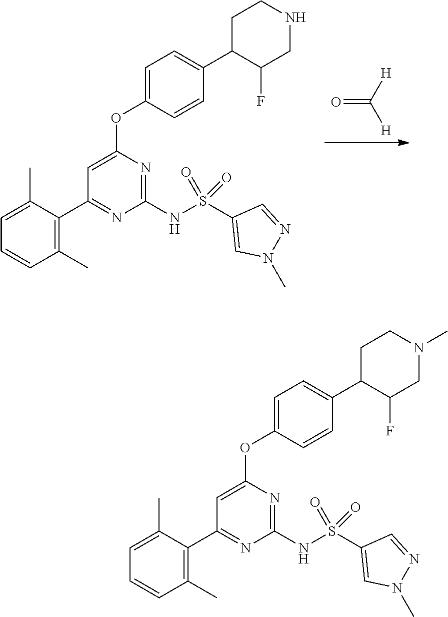 [SMILES: Cc1cccc(C)c1-c1cc(Oc2ccc(C3CCNCC3F)cc2)nc(NS(=O)(=O)c2cnn(C)c2)n1 ; C=O ; Cc1cccc(C)c1-c1cc(Oc2ccc(C3CCN(C)CC3F)cc2)nc(NS(=O)(=O)c2cnn(C)c2)n1]

A solution of N-[4-(2,6-dimethylphenyl)-6-[4-(3-fluoro-4-piperidyl)phenoxy]pyrimidin-2-yl]-1-methyl-pyrazole-4-sulfonamide (diastereomeric mixture, 12 mg, 0.02236 mmol) in formic acid (100 μL, 2.651 mmol) and formaldehyde (100 μL of 37 % w/v, 1.232 mmol) was stirred at 60° C. for 24 hours. The reaction mixture was diluted with MeOH and purification by HPLC (1-99% ACN in water (HCl modifier)) gave N-[4-(2,6-dimethylphenyl)-6-[4-(3-fluoro-1-methyl-4-piperidyl)phenoxy]pyrimidin-2-yl]-1-methyl-pyrazole-4-sulfonamide (hydrochloride salt) (7.4 mg, 56%). ESI-MS m/z calc. 550.21625, found 551.4 (M+1)⁺; Retention time: 1.15 minutes; LC method A.

### Example 429: Preparation of Compound 1460

### Step 1: 4-(4-Methylmorpholin-2-yl)phenol

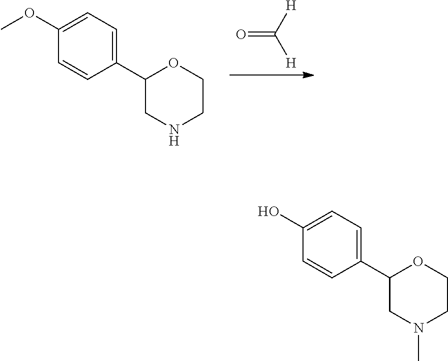

A MeOH (0.2 mL) solution of 2-(4-methoxyphenyl)morpholine (70 mg, 0.36 mmol), AcOH (200 μL, 3.52 mmol), formaldehyde (200 μL, 7.26 mmol), and sodium triacetoxyborohydride (178.1 mg, 0.8403 mmol) was stirred at room temperature for 90 minutes and then concentrated in vacuo. The residue was diluted in DCM (10 mL) and washed with a saturated aqueous solution of sodium bicarbonate. The DCM layer was separated, dried over magnesium sulfate, filtered and concentrated in vacuo. The resulting residue was dissolved in DCM (1.5 mL) and cooled to 0° C. with an ice-water bath. BBr3 (1.1 mL of 1 M, 1.100 mmol) was slowly added and the reaction mixture was warmed to room temperature, upon which the product precipitated out of solution. The entire mixture was filtered and the precipitate was washed with DCM (5 mL) and then dried under high vacuum for 16 hours to give 4-(4-methylmorpholin-2-yl)phenol (hydrobromide salt) (68.2 mg, 17%). ESI-MS m/z calc. 193.11028, found 194.27 (M+1)⁺; Retention time: 0.33 minutes (LC method J).

### Step 2: N-[4-(2,6-Dimethylphenyl)-6-[4-(4-methylmorpholin-2-yl)phenoxy]pyrimidin-2-yl]-1-methyl-pyrazole-4-sulfonamide

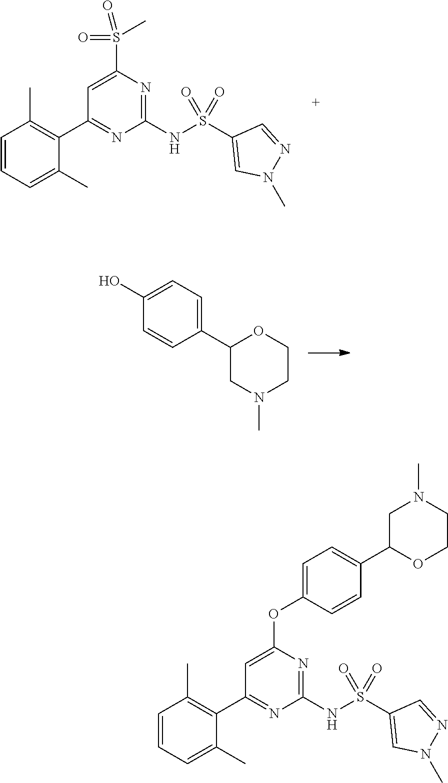 [SMILES: Cc1cccc(C)c1-c1cc(S(C)(=O)=O)nc(NS(=O)(=O)c2cnn(C)c2)n1 ; CN1CCOC(c2ccc(O)cc2)C1 ; Cc1cccc(C)c1-c1cc(Oc2ccc(C3CN(C)CCO3)cc2)nc(NS(=O)(=O)c2cnn(C)c2)n1]

An NMP (0.6 mL) mixture of N-[4-(2,6-dimethylphenyl)-6-methylsulfonyl-pyrimidin-2-yl]-1-methyl-pyrazole-4-sulfonamide (9.3 mg, 0.022 mmol), 4-(4-methylmorpholin-2-yl)phenol (hydrobomide salt) (16.1 mg, 0.0294 mmol), and cesium carbonate (80.2 mg, 0.246 mmol) was stirred at 110° C. for 1 hour and then cooled to room temperature. The solution was filtered and the resulting residues dissolved in 0.8 mL MeOH, and purified by reverse phase chromatography using a 15 minute gradient of 20% MeCN in water to 80% MeCN (HCl modifier) to give N-[4-(2,6-dimethylphenyl)-6-[4-(4-methylmorpholin-2-yl)phenoxy]pyrimidin-2-yl]-1-methyl-pyrazole-4-sulfonamide (hydrochloride salt) (3.8 mg, 30%). ¹H NMR (400 MHz, DMF-d₇) δ 10.76 (s, 1H), 7.63-7.48 (m, 3H), 7.44-7.36 (m, 2H), 7.31-7.21 (m, 1H), 7.21-7.10 (m, 3H), 6.65 (s, 1H), 4.87 (dd, J=11.1, 2.2 Hz, 1H), 4.20 (dd, J=12.8, 3.6 Hz, 1H), 3.95 (td, J=12.5, 2.2 Hz, 1H), 3.74 (s, 3H), 3.66 (d, J=12.5 Hz, 1H), 3.46 (d, J=12.3 Hz, 1H), 3.26-3.06 (m, 2H), 2.83 (d, J=4.4 Hz, 3H), 2.05 (s, 6H). ESI-MS m/z calc. 534.2049, found 535.55 (M+1)⁺; Retention time: 1.17 minutes; LC method A.

### Example 430: Preparation of Compound 1461

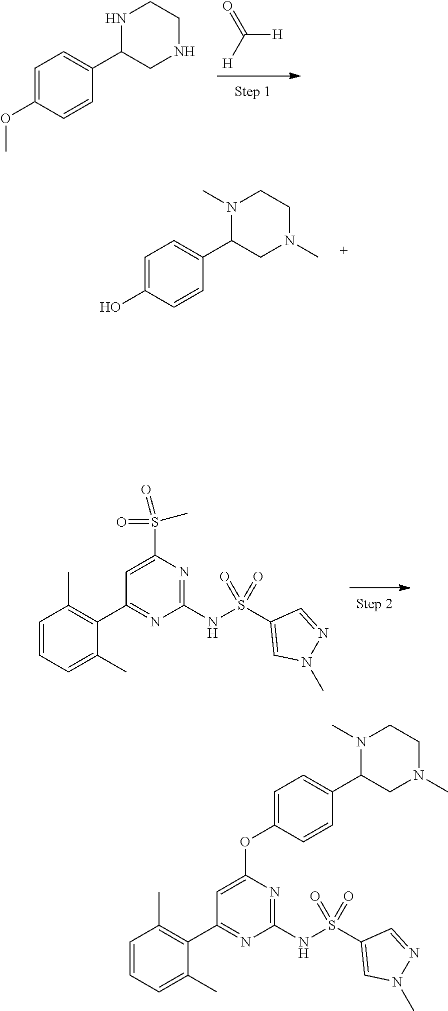

### Step 1: 4-(1,4-Dimethylpiperazin-2-yl)phenol

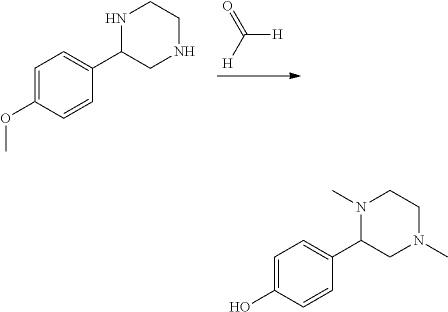

A concentrated solution of HBr (1.0 mL, 18.42 mmol) of 2-(4-methoxyphenyl)piperazine (approximately 48.70 mg, 0.2533 mmol) was stirred at 90° C. for 18 hours and then cooled to room temperature. The suspension was filtered, and the wet cake was washed with acetonitrile (0.5 mL) followed by diethyl ether (1 mL) and dried under suction vacuum for 1 hour. The resulting solid was taken up in formaldehyde (200 μL, 7.260 mmol) and formic acid (200 μL, 5.301 mmol) and heated to 90° C. for 3 hours and then cooled to room temperature and concentrated in vacuo to give 4-(1,4-dimethylpiperazin-2-yl)phenol (Hydrobromic Acid (2)) (30 mg, 32%). ESI-MS m/z calc. 206.1419, found 207.26 (M+1)⁺; Retention time: 0.1 minutes; LC method D.

### Step 2: N-[4-(2,6-Dimethylphenyl)-6-[4-(1,4-dimethylpiperazin-2-yl)phenoxy]pyrimidin-2-yl]-1-methyl-pyrazole-4-sulfonamide

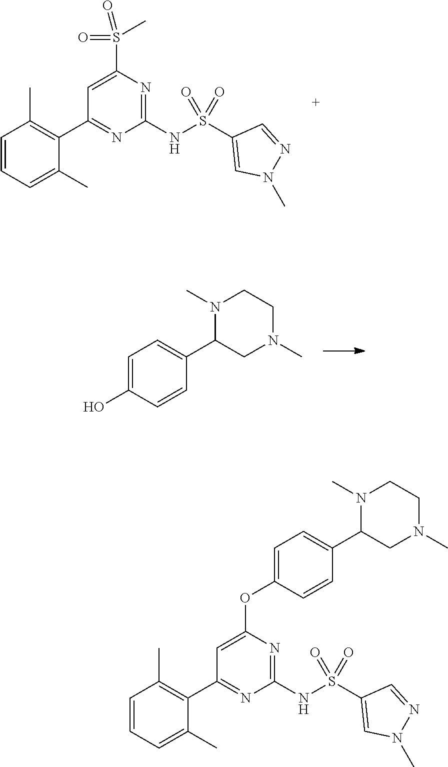 [SMILES: Cc1cccc(C)c1-c1cc(S(C)(=O)=O)nc(NS(=O)(=O)c2cnn(C)c2)n1 ; CN1CCN(C)C(c2ccc(O)cc2)C1 ; Cc1cccc(C)c1-c1cc(Oc2ccc(C3CN(C)CCN3C)cc2)nc(NS(=O)(=O)c2cnn(C)c2)n1]

A NMP (0.5 mL) mixture of N-[4-(2,6-dimethylphenyl)-6-methylsulfonyl-pyrimidin-2-yl]-1-methyl-pyrazole-4-sulfonamide (9.7 mg, 0.02301 mmol), 4-(1,4-dimethylpiperazin-2-yl)phenol (approximately 7.121 mg, 0.03452 mmol), and Cs₂CO₃ (71 mg, 0.2179 mmol) was heated to 110° C. for 60 minutes and then cooled to room temperature. The solution was filtered and the resulting residue dissolved in 0.8 mL MeOH, and purified by reverse phase chromatography using a 30 minute gradient of 20% MeCN in water to 80% MeCN (HCl modifier) to give N-[4-(2,6-dimethylphenyl)-6-[4-(1,4-dimethylpiperazin-2-yl)phenoxy]pyrimidin-2-yl]-1-methyl-pyrazole-4-sulfonamide (dihydrochloride salt) (1.2 mg, 8%). ESI-MS m/z calc. 547.2366, found 548.54 (M+1)⁺; Retention time: 1.1 minutes; LC method A.

### Example 431: Preparation of Compound 1462 and Compound 1463

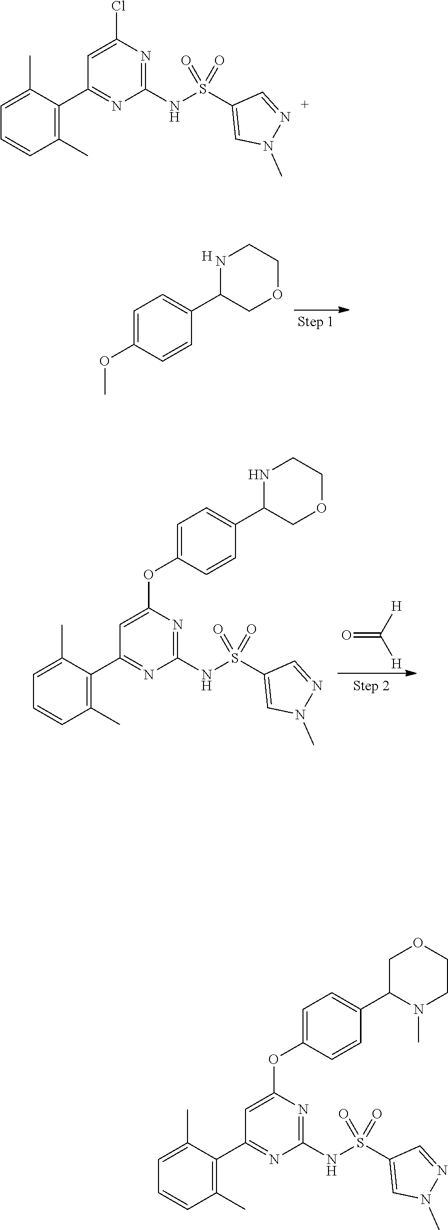

### Step 1: N-[4-(2,6-Dimethylphenyl)-6-(4-morpholin-3-ylphenoxy)pyrimidin-2-yl]-1-methyl-pyrazole-4-sulfonamide

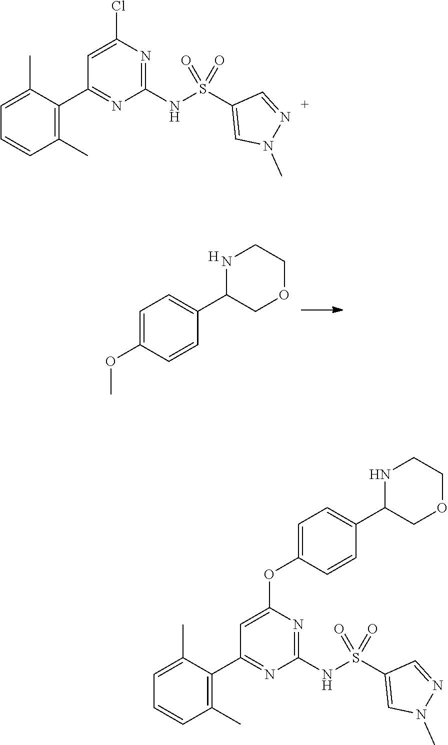

In a glass vial 3-(4-methoxyphenyl)morpholine (28.3 mg, 0.1464 mmol) and HBr (200 μL, 3.683 mmol) were combined and the mixture was stirred at 80° C. for 4 hours and then cooled to room temperature and concentrated in vacuo. The resulting residue was triturated in diethyl ether and then filtered by vacuum suction. The resulting solid was added to an NMP (0.7 mL) mixture of N-[4-chloro-6-(2,6-dimethylphenyl)pyrimidin-2-yl]-1-methyl-pyrazole-4-sulfonamide (29.7 mg, 0.0786 mmol) and Cs₂CO₃ (116 mg, 0.3560 mmol) and heated to 110° C. for 5 hours and then cooled to room temperature. The solution was filtered and the resulting residues dissolved in 0.8 mL MeOH, and purified by reverse phase chromatography using a 15 minute gradient of 1% MeCN in water to 99% MeCN (HCl modifier) to give N-[4-(2,6-dimethylphenyl)-6-(4-morpholin-3-ylphenoxy)pyrimidin-2-yl]-1-methyl-pyrazole-4-sulfonamide (hydrochloride salt) (3.5 mg, 8%). ¹H NMR (400 MHz, DMSO-d₆) δ 9.66 (s, 1H), 7.74 (d, J=8.7 Hz, 2H), 7.46 (d, J=8.6 Hz, 2H), 7.30-7.22 (m, 1H), 7.19 (s, 1H), 7.14 (d, J=7.6 Hz, 2H), 6.64 (s, 1H), 4.67-4.51 (m, 1H), 4.10-3.97 (m, 2H), 3.93-3.79 (m, 2H), 3.75 (s, 3H), 3.42-3.23 (m, 2H), 2.04 (s, 6H). ESI-MS m/z calc. 520.1893, found 521.54 (M+1)⁺; Retention time: 1.12 minutes; LC method A.

### Step 2: N-[4-(2,6-Dimethylphenyl)-6-[4-(4-methylmorpholin-3-yl)phenoxy]pyrimidin-2-yl]-1-methyl-pyrazole-4-sulfonamide

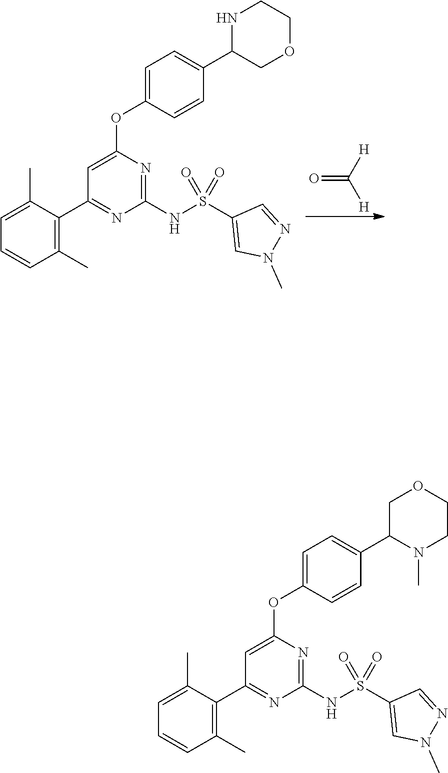 [SMILES: Cc1cccc(C)c1-c1cc(Oc2ccc(C3COCCN3)cc2)nc(NS(=O)(=O)c2cnn(C)c2)n1 ; C=O ; Cc1cccc(C)c1-c1cc(Oc2ccc(C3COCCN3C)cc2)nc(NS(=O)(=O)c2cnn(C)c2)n1]

In a glass vial N-[4-(2,6-dimethylphenyl)-6-(4-morpholin-3-ylphenoxy)pyrimidin-2-yl]-1-methyl-pyrazole-4-sulfonamide (hydrochloride salt) (5.5 mg, 0.01056 mmol), formic acid (850 μL, 22.53 mmol), and formaldehyde (850 μL, 30.86 mmol) were mixed and heated under stirring at 90° C. for 1 hour and then cooled to room temperature. The solution was filtered and the resulting residues purified by reverse phase chromatography using a 15 min gradient of 1% MeCN in water to 99% MeCN (HCl modifier) to give N-[4-(2,6-dimethylphenyl)-6-[4-(4-methylmorpholin-3-yl)phenoxy]pyrimidin-2-yl]-1-methyl-pyrazole-4-sulfonamide (hydrochloride salt) (2.4 mg, 40%). ¹H NMR (400 MHz, DMSO-d₆) δ 11.38 (s, 1H), 7.90-7.77 (m, 3H), 7.48 (d, 2H), 7.29-7.21 (m, 1H), 7.14 (d, J=7.6 Hz, 2H), 7.08 (s, 1H), 6.70 (s, 1H), 4.58-4.49 (m, 1H), 4.16-3.83 (m, 6H), 3.78 (s, 3H), 2.58 (d, J=4.3 Hz, 3H), 2.05 (s, 6H). ESI-MS m/z calc. 534.2049, found 535.52 (M+1)⁺; Retention time: 1.13 minutes; LC method A.

### Example 432: Preparation of Compound 1464 and Compound 1465

### Step 1: N-[4-(2,6-Dimethylphenyl)-6-[4-(1,4-dimethylpiperazin-2-yl)phenoxy]pyrimidin-2-yl]-1-methyl-pyrazole-4-sulfonamide, enantiomer 1, (Compound 1464) and N-[4-(2,6-dimethylphenyl)-6-[4-(1,4-dimethylpiperazin-2-yl)phenoxy]pyrimidin-2-yl]-1-methyl-pyrazole-4-sulfonamide, enantiomer 2 (Compound 1465)

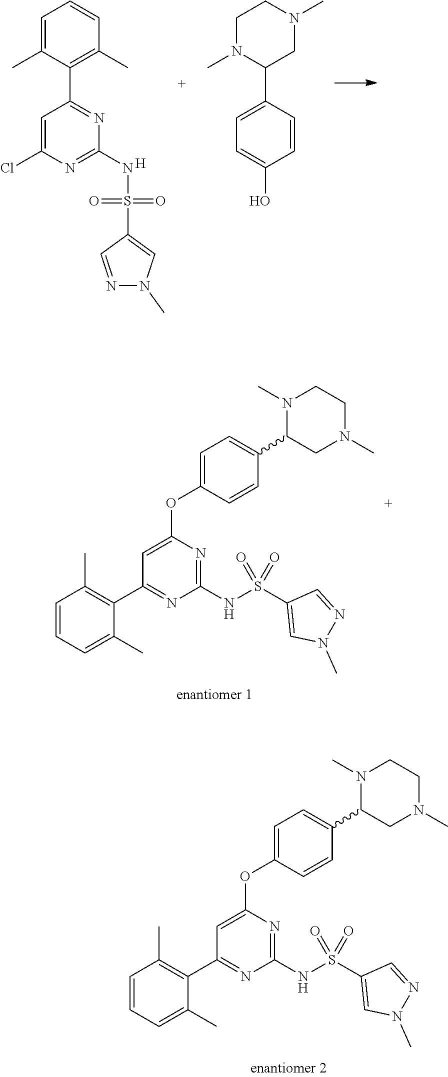

An NMP (0.8 mL) solution of 4-(1,4-dimethylpiperazin-2-yl)phenol (hydrobromide salt) (72 mg, 0.25 mmol), N-[4-chloro-6-(2,6-dimethylphenyl)pyrimidin-2-yl]-1-methyl-pyrazole-4-sulfonamide (60 mg, 0.16 mmol) and cesium carbonate (352.7 mg, 1.083 mmol) was heated to 110° C. for 20 hours and then cooled to room temperature. The solution was filtered and the resulting residue dissolved in 0.8 mL MeOH, and purified by reverse phase chromatography using a 15 minute gradient of 20% MeCN in water to 80% MeCN (HCl modifier) to afford 70 mg of racemic N-[4-(2,6-dimethylphenyl)-6-[4-(1,4-dimethylpiperazin-2-yl)phenoxy]pyrimidin-2-yl]-1-methyl-pyrazole-4-sulfonamide. The two enantiomers are separated by SFC using the following conditions: ChiralCel OD-H (250×10 mm), 5 m column; eluant: 15% MeOH (20 mM NH₃), 85% C₀₂; flow: 70 mL/min; concentration: −32 mg/mL in methanol; injection volume: 70 μL; pressure: 100 bar; wavelength 220 nm.

Enantiomer 1 (peak 1): N-[4-(2,6-dimethylphenyl)-6-[4-(1,4-dimethylpiperazin-2-yl)phenoxy]pyrimidin-2-yl]-1-methyl-pyrazole-4-sulfonamide (hydrochloride salt) (6.6 mg, 7%)

¹H NMR (400 MHz, DMSO-d₆) δ 7.97-7.74 (m, 3H), 7.55-7.46 (m, 2H), 7.28-7.23 (m, 1H), 7.20 (s, 1H), 7.14 (d, J=7.6 Hz, 2H), 6.70 (s, 1H), 4.45-3.18 (m, 9H), 2.87 (s, 3H), 2.51 (s, 3H), 2.05 (s, 6H) (2H proton likely overlapped under DMSO signal). ESI-MS m/z calc. 547.2366, found 548.58 (M+1)⁺; Retention time: 1.07 minutes, (LC method A).

Enantiomer 2 (peak 2): N-[4-(2,6-dimethylphenyl)-6-[4-(1,4-dimethylpiperazin-2-yl)phenoxy]pyrimidin-2-yl]-1-methyl-pyrazole-4-sulfonamide (hydrochloride salt) (4.7 mg, 5%) H NMR (400 MHz, DMSO-d₆) δ 7.94-7.61 (m, 3H), 7.52-7.45 (m, 2H), 7.29-7.21 (m, 1H), 7.18-7.11 (m, 3H), 6.69 (s, 1H), 3.78 (s, 3H), 2.83 (s, 3H), 2.05 (s, 6H), (11H likely overlapped under water and DMSO signal). ESI-MS m/z calc. 547.2366, found 548.58 (M+1)⁺; Retention time: 1.08 minutes, (LC method A).

### Example 433: Preparation of Compound 1466

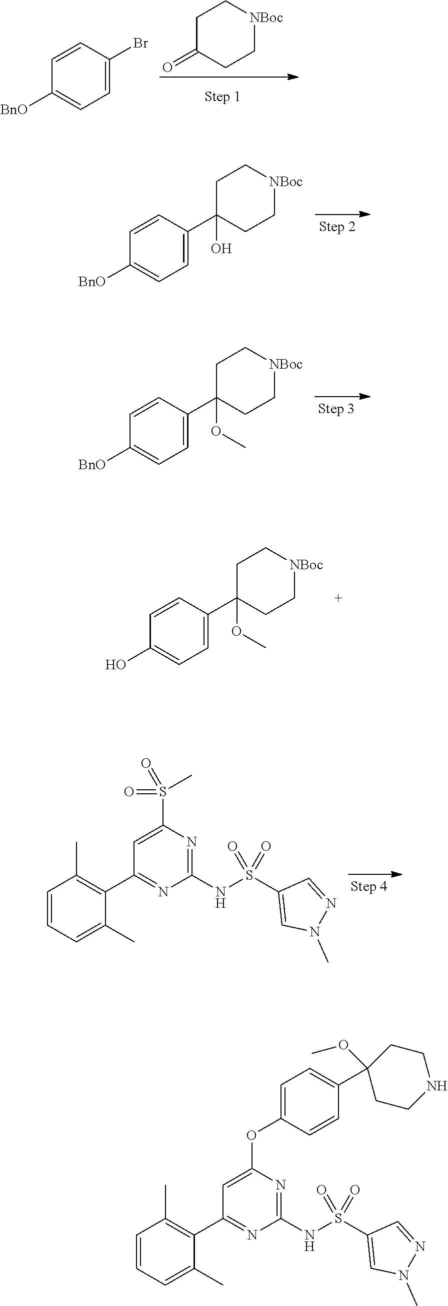

### Step 1: tert-Butyl 4-(4-benzyloxyphenyl)-4-hydroxy-piperidine-1-carboxylate

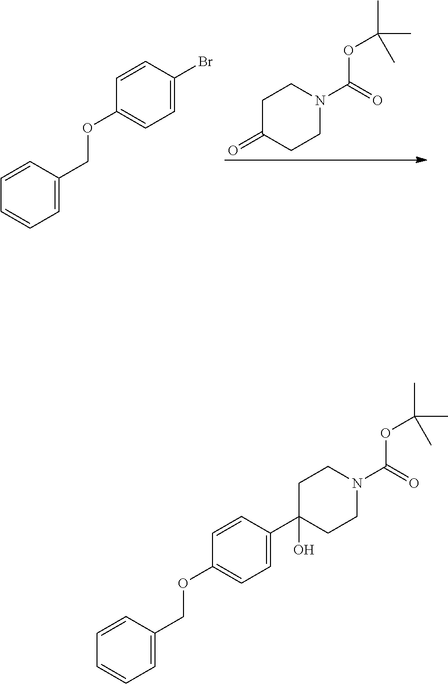 [SMILES: Brc1ccc(OCc2ccccc2)cc1 ; CC(C)(C)OC(=O)N1CCC(=O)CC1 ; CC(C)(C)OC(=O)N1CCC(O)(c2ccc(OCc3ccccc3)cc2)CC1]

In a 100 mL round-bottomed flask equipped with a stir bar, 1-benzyloxy-4-bromo-benzene (2.1103 g, 8.020 mmol) was dissolved in tetrahydrofuran (12.0 mL) and cooled to −78° C. A solution of n-BuLi (6.0 mL of 1.6 M, 9.600 mmol) in hexanes was added dropwise, and this yellow slurry was stirred at −78° C. for 30 minute under a nitrogen atmosphere. A solution of tert-butyl 4-oxopiperidine-1-carboxylate (1.5702 g, 7.881 mmol) in tetrahydrofuran (12.0 mL) was then added dropwise. This yellow transparent solution was then stirred at −78° C. for 2 hour. The reaction mixture was quenched onto a saturated aqueous ammonium chloride solution (20mL). The mixture was extracted with ethyl acetate (3×30 mL). The combined organic extracts were washed with water (60 mL) and saturated aqueous sodium chloride solution (60 mL), then dried over sodium sulfate, filtered, and evaporated in vacuo. Purification by silica gel chromatography (40 g of silica, 0 to 40% gradient of ethyl acetate/hexanes) gave 1.7984 g of a white chunky solid, tert-butyl 4-(4-benzyloxyphenyl)-4-hydroxy-piperidine-1-carboxylate (1.7984 g, 60%)¹H NMR (400 MHz, dimethylsulfoxide-d₆) δ 7.46-7.41 (m, 2H), 7.41-7.35 (m, 4H), 7.35-7.29 (m, 1H), 6.97-6.91 (m, 2H), 5.08 (s, 2H), 4.98 (s, 1H, D20 exchangeable), 3.95-3.68 (bs, 2H), 3.27-2.95 (bs, 2H), 1.75 (td, J=13.0, 4.7 Hz, 2H), 1.56 (d, J=12.2 Hz, 2H), 1.41 (s, 9H). ESI-MS m/z calc. 383.20966, found 366.2 (M+1-H₂O)⁺; Retention time: 0.71 minutes; LC method D.

### Step 2: tert-Butyl 4-(4-benzyloxyphenyl)-4-methoxy-piperidine-1-carboxylate

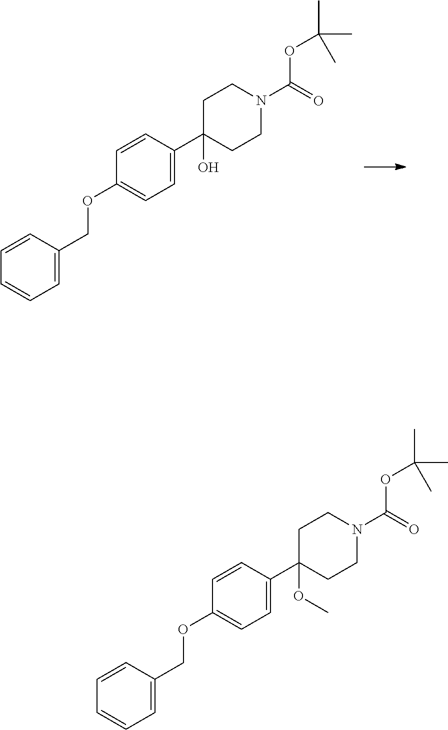 [SMILES: CC(C)(C)OC(=O)N1CCC(O)(c2ccc(OCc3ccccc3)cc2)CC1 ; COC1(c2ccc(OCc3ccccc3)cc2)CCN(C(=O)OC(C)(C)C)CC1]

In a 25 mL round-bottomed flask equipped with a stir bar, tert-butyl 4-(4-benzyloxyphenyl)-4-hydroxy-piperidine-1-carboxylate (702.1 mg, 1.831 mmol) was dissolved in tetrahydrofuran (5.0 mL) and cooled to 0° C. 60% NaH (168.3 mg, 4.208 mmol) was added in one portion, and this slurry was stirred at 0° C. for 30 minutes. Then, iodomethane (200 μL, 3.213 mmol) was added in one portion, and the reaction mixture was stirred at 0° C. for 30 minutes. TLC analysis indicated reaction completion, and therefore the reaction mixture was quenched with saturated aqueous ammonium chloride solution (8 mL). The mixture was extracted with ethyl acetate (3×10 mL). The combined organic extracts was washed with water (20 mL) and saturated aqueous sodium chloride solution (20 mL), then dried over sodium sulfate, filtered, and evaporated in vacuo to give a very viscous, clear liquid that crystallized under high vacuum: tert-butyl 4-(4-benzyloxyphenyl)-4-methoxy-piperidine-1-carboxylate (701.0 mg, 96%)¹H NMR (400 MHz, dimethylsulfoxide-d₆) δ 7.48-7.43 (m, 2H), 7.42-7.37 (m, 2H), 7.36-7.32 (m, 1H), 7.32-7.26 (m, 2H), 7.03-6.97 (m, 2H), 5.09 (s, 2H), 3.94-3.64 (bs, 2H), 3.22-2.90 (bs, 2H), 2.84 (s, 3H), 1.95 (d, J=12.4 Hz, 2H), 1.70 (td, J=13.4, 4.7 Hz, 2H), 1.40 (s, 9H). ESI-MS m/z calc. 397.2253, found 366.2 (M+1-OMe)⁺; Retention time: 0.79 minutes; LC method D.

### Step 3: tert-Butyl 4-(4-hydroxyphenyl)-4-methoxy-piperidine-1-carboxylate and tert-butyl 4-(4-hydroxyphenyl)piperidine-1-carboxylate

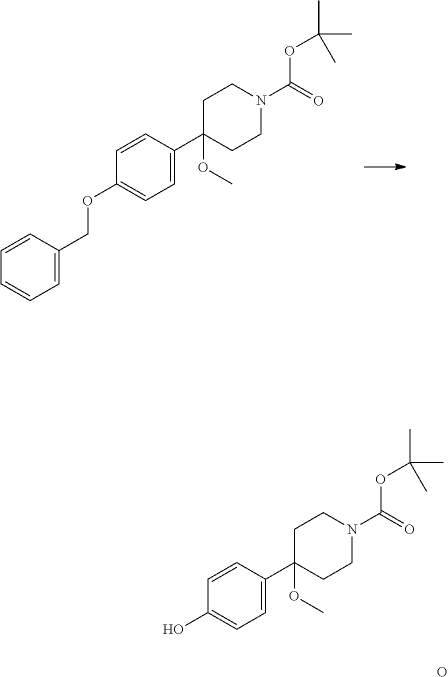 [SMILES: COC1(c2ccc(OCc3ccccc3)cc2)CCN(C(=O)OC(C)(C)C)CC1 ; COC1(c2ccc(O)cc2)CCN(C(=O)OC(C)(C)C)CC1 ; O]

In a 10 mL vial equipped with a magnetic stir bar, tert-butyl 4-(4-benzyloxyphenyl)-4-methoxy-piperidine-1-carboxylate (41.2 mg, 0.1036 mmol) was dissolved in methanol (2.0 mL), and this solution was purged with a balloon of hydrogen gas for 5 minutes. The cap was briefly removed, and 10% Pd/C (5.1 mg, 0.004792 mmol) was added. This reaction mixture was stirred under hydrogen gas (2 L, 79.37 mmol) at room temperature for 16 hours. It was then filtered through Celite and rinsed with methanol (5.0 mL). This solution was evaporated in vacuo to give 39.1 mg of a slightly yellow oil. This material was purified by preparative TLC (two full silica plates, 20 cm×20 cm, 250 m thickness, 60 Å particle size, 50% ethyl acetate/hexanes, UV active bands) to give two products, both of which were white solids: tert-butyl 4-(4-hydroxyphenyl)-4-methoxy-piperidine-1-carboxylate (5.5 mg, 17%) and tert-butyl 4-(4-hydroxyphenyl)piperidine-1-carboxylate (20.0 mg, 70%).

### Step 4: N-[4-(2,6-Dimethylphenyl)-6-[4-(4-methoxy-4-piperidyl)phenoxy]pyrimidin-2-yl]-1-methyl-pyrazole-4-sulfonamide

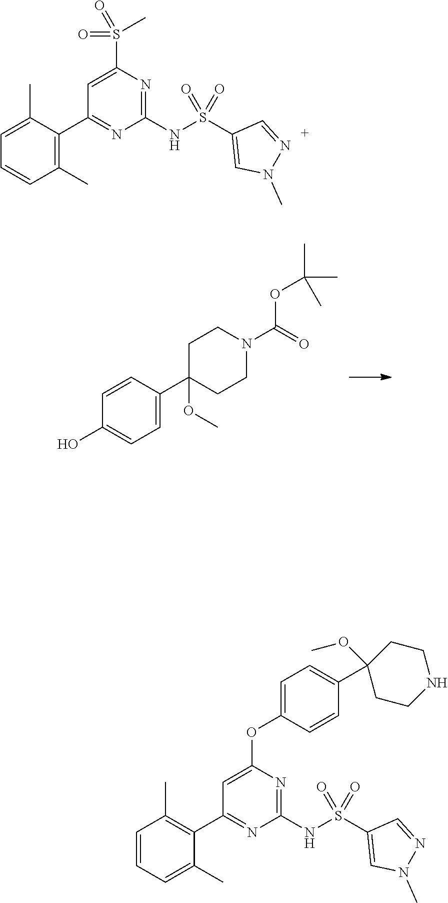

Stage 1: To a 3 mL vial equipped with a magnetic stir bar, N-[4-(2,6-dimethylphenyl)-6-methylsulfonyl-pyrimidin-2-yl]-1-methyl-pyrazole-4-sulfonamide (17.2 mg, 0.04081 mmol), tert-butyl 4-(4-hydroxyphenyl)-4-methoxy-piperidine-1-carboxylate (11.8 mg, 0.03839 mmol) and N-methylpyrrolidinone (700 μL) were added, followed by cesium carbonate (35.2 mg, 0.1080 mmol). This mixture was stirred at 60° C. for 13 hours. The reaction mixture was then cooled to room temperature, quenched with 1 N HCl (1 mL), and extracted with ethyl acetate (3×1 mL). The combined organic extracts were washed with water (2×2 mL) and saturated aqueous sodium chloride solution (2 mL), then dried over sodium sulfate, filtered, and evaporated in vacuo to give 22 mg of crude product. This material was purified by preparative TLC (two full silica plates, 20 cm×20 cm, 250 m thickness, 60 Å particle size, 50% ethyl acetate/hexanes, UV active bands) to give 19.5 mg of ˜85% pure product.

Stage 2: The product from Stage 1 was dissolved in dioxane (300 μL), to which was added a solution of HCl in dioxane (300 μL of 4 M, 1.200 mmol). This solution was stirred at 70° C. for 15 minutes, after which it was cooled to room temperature, filtered, and purified by reverse phase HPLC (1-99% acetonitrile in water using HCl as a modifier) to give N-[4-(2,6-dimethylphenyl)-6-[4-(4-methoxy-4-piperidyl)phenoxy]pyrimidin-2-yl]-1-methyl-pyrazole-4-sulfonamide (hydrochloride salt) (3.9 mg, 17%). ESI-MS m/z calc. 548.2206, found 549.4 (M+1)⁺; Retention time: 1.22 minutes; LC method A.

### Example 434: Preparation of Compound 1467

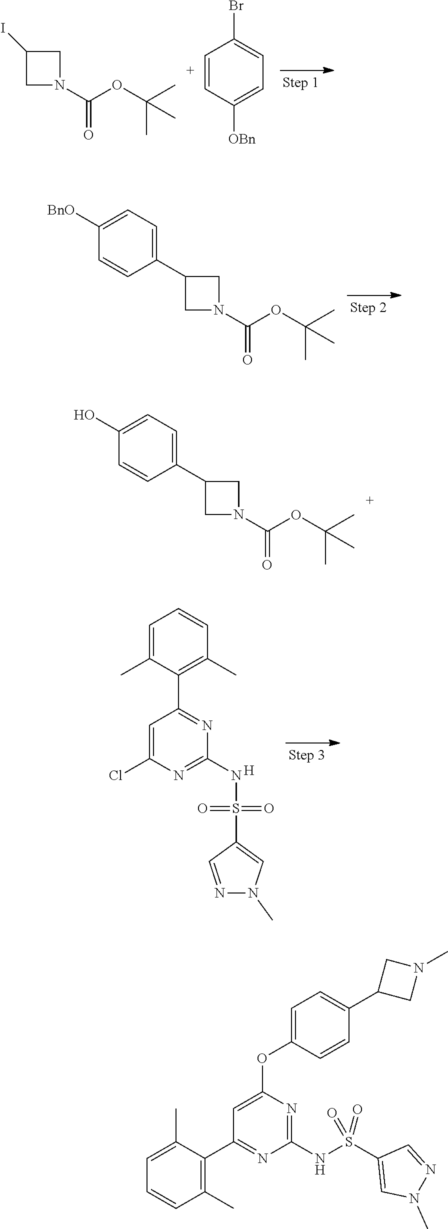

### Step 1: tert-Butyl 3-(4-benzyloxyphenyl)azetidine-1-carboxylate

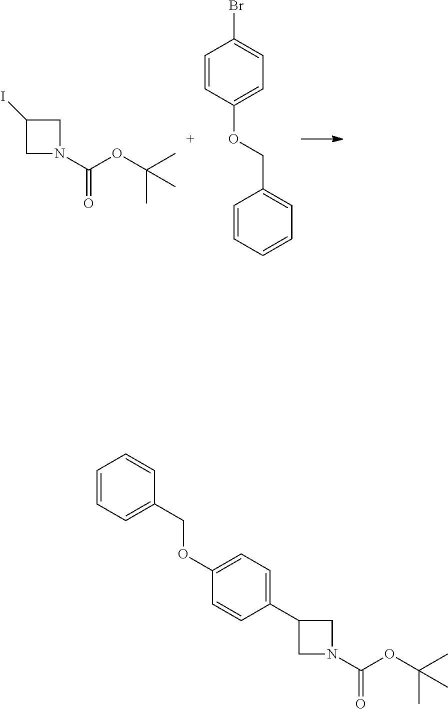 [SMILES: CC(C)(C)OC(=O)N1CC(I)C1 ; Brc1ccc(OCc2ccccc2)cc1 ; CC(C)(C)OC(=O)N1CC(c2ccc(OCc3ccccc3)cc2)C1]

To a suspension of zinc (251.2 mg, 3.840 mmol) in THE (2 mL) was added 1,2-dibromoethane (approximately 35.69 mg, 16.37 μL, 0.1900 mmol). The mixture was heated to 65° C. and allowed to cool to room temperature over 30 minutes. Chloro(trimethyl)silane (approximately 20.64 mg, 24.11 μL, 0.1900 mmol) was then added, and the mixture was stirred at room temperature for an additional 30 minutes. A solution of tert-butyl 3-iodoazetidine-1-carboxylate (809 mg, 2.858 mmol) in THE (0.8 mL) was added dropwise to the reaction mixture and the mixture was stirred 1 hour at room temperature. The mixture was quickly filtered through a syringe filter into a vessel containing 1-benzyloxy-4-bromo-benzene (500 mg, 1.900 mmol), Pd(dppf)Cl₂ (approximately 69.54 mg, 0.09504 mmol) and copper iodide (approximately 181.0 mg, 0.9504 mmol). The reaction mixture was then stirred at 70° C. for 4 hours. The reaction mixture was filtered through Celite and concentrated to ⅓ of the volume under reduced pressure. To the remaining black solution was added EtOAc (25 mL) and a saturated aqueous sodium chloride solution (10 mL). The organic portion was concentrated to dryness under reduced pressure. The residue was purified via silica gel using 0 to 10% EtOAc in hexane to obtain tert-butyl 3-(4-benzyloxyphenyl)azetidine-1-carboxylate (32 mg, 5%). ¹H NMR (400 MHz, Chloroform-d) δ 7 7.47-7.29 (m, 5H), 7.25-7.21 (m, 2H), 7.00-6.92 (m, 2H), 5.06 (s, 2H), 4.30 (t, J=8.7 Hz, 2H), 3.97-3.89 (m, 2H), 3.74-3.62 (m, 1H), 1.46 (s, 9H). ESI-MS m/z calc. 339.18344, found 284.34 (M+1-56)⁺; Retention time: 3.42 minutes.

### Step 2: tert-Butyl 3-(4-hydroxyphenyl)azetidine-1-carboxylate

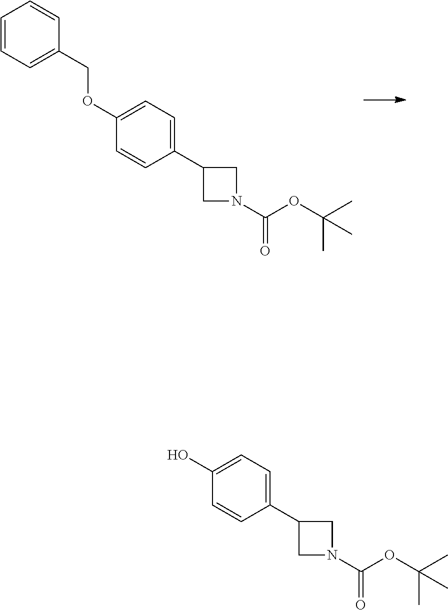 [SMILES: CC(C)(C)OC(=O)N1CC(c2ccc(OCc3ccccc3)cc2)C1 ; CC(C)(C)OC(=O)N1CC(c2ccc(O)cc2)C1]

In a glass vial was tert-butyl 3-(4-benzyloxyphenyl)azetidine-1-carboxylate (32.1 mg, 0.0946 mmol), EtOH (2 mL), ethyl acetate (2 mL), and dihydroxypalladium (45.7 mg, 0.0651 mmol) mixed and the mixture was sparged with nitrogen (three times) and then reduced under an atmosphere of H₂ using a hydrogen balloon for 16 hours. The catalyst was filtered off and the resulting filtrate was concentrated in vacuo to give tert-butyl 3-(4-hydroxyphenyl)azetidine-1-carboxylate (23 mg, 98%). The material was used without any further purification. ¹H NMR (400 MHz, Chloroform-d) δ 7.18 (d, 1H), 7.06 (d, J=8.3 Hz, 1H), 6.85-6.75 (m, 2H), 4.30 (t, J=8.7 Hz, 2H), 3.92 (dd, J=8.6, 6.0 Hz, 2H), 3.72-3.60 (m, 1H), 1.47 (s, 9H). ESI-MS m z calc. 249.13649, found 196.26 (M+1)⁺; Retention time: 1.38 minutes; LC method A.

### Step 3: N-[4-[4-(azetidin-3-yl)phenoxy]-6-(2,6-dimethylphenyl)pyrimidin-2-yl]-1 -methyl-pyrazole-4-sulfonamide Compound 992 and N-[4-(2,6-dimethylphenyl)-6-[4-(1-methylazetidin-3-yl)phenoxy]pyrimidin-2-yl]-1-methyl-pyrazole-4- sulfonamide Compound 1467

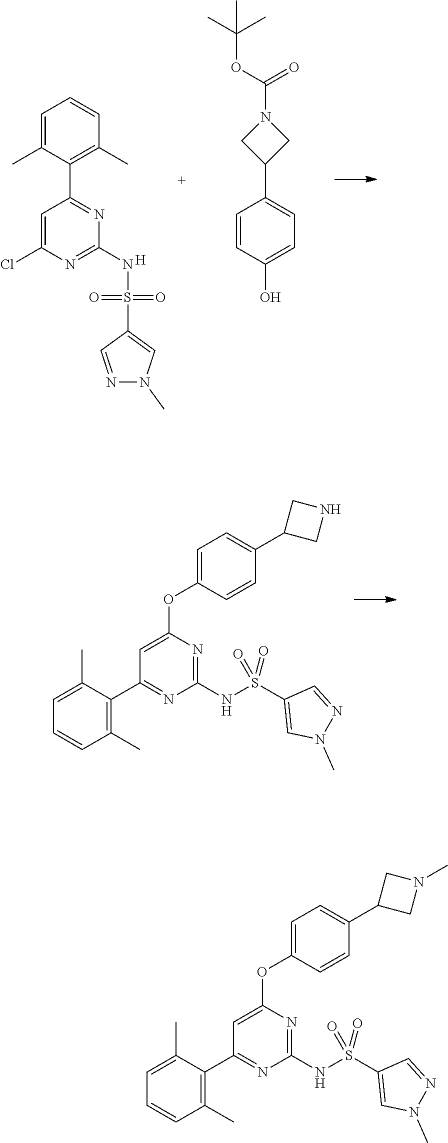

Stage 1: In a glass vial NMP (600 μL), tert-butyl 3-(4-hydroxyphenyl)azetidine-1-carboxylate (23 mg, 0.092 mmol), N-[4-chloro-6-(2,6-dimethylphenyl)pyrimidin-2-yl]-1 -methyl-pyrazole-4-sulfonamide (20.7 mg, 0.0548 mmol) and Cs₂CO₃ (58.5 mg, 0.1795 mmol) were mixed and the reaction was stirred at 110° C. for 16 hours. After cooling to room temperature, the mixture was filtered and the resulting residue diluted in 0.8 mL DMSO, and injected onto a reverse-phase preparative HPLC for chromatography using a 30 min gradient of 20% MeCN in water to 80% MeCN with HCl as a modifier, to give the desired intermediate. After concentration in vacuo the residue was treated with DCM (0.8 mL) and TFA (200 μL, 2.60 mmol) at room temperature for 2 hours and then concentrated in vacuo. The residue was dissolved in 0.8 mL DMSO, and purified by reverse phase chromatography using a 15 minute gradient of 1% MeCN in water to 99% MeCN (HCl modifier) to afford N-[4-[4-(azetidin-3-yl)phenoxy]-6-(2,6-dimethylphenyl)pyrimidin-2-yl]-1-methyl-pyrazole-4-sulfonamide (hydrochloride salt) (3.6 mg, 13%). ¹H NMR (400 MHz, DMSO-d₆) δ 9.09 (s, 1H), 8.73 (s, 1H), 7.63-7.54 (m, 2H), 7.37 (d, J=8.5 Hz, 2H), 7.25 (t, J=7.6 Hz, 1H), 7.14 (d, J=7.6 Hz, 2H), 6.63 (s, 1H), 4.46-3.99 (m, 5H), 3.74 (s, 3H), 2.04 (s, 6H). ESI-MS m/z calc. 490.1787, found 491.5 (M+1)⁺; Retention time: 1.12 minutes. LC method A.

Stage 2: Some of the product from stage 1 was treated with formaldehyde (200 μL, 7.260 mmol) and formic acid (200 μL, 5.30 mmol at 90° C. for 1 hour. The solution was filtered and the resulting residue diluted in 0.8 mL DMSO and purified by reverse phase chromatography using a 15 min gradient of 20% MeCN in water to 80% MeCN (HCl modifier) to afford of N-[4-(2,6-dimethylphenyl)-6-[4-(1-methylazetidin-3-yl)phenoxy]pyrimidin-2-yl]-1 -methyl-pyrazole-4-sulfonamide (hydrochloride salt) (1.1 mg, 4%). ESI-MS m/z calc. 504.19437, found 505.54 (M+1)⁺; Retention time: 1.14 minutes; LC method A.

### Example 435: Preparation of Compound 1468

### Step 1: N-[4-[4-[2-(dimethylamino)ethyl]phenoxy]-6-(2,6-dimethylphenyl)pyrimidin-2-yl]-1-methyl-pyrazole-4-sulfonamide

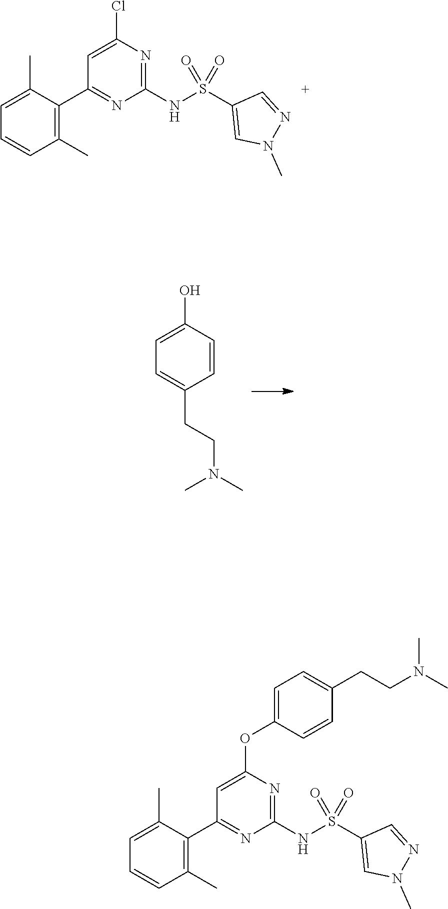 [SMILES: Cc1cccc(C)c1-c1cc(Cl)nc(NS(=O)(=O)c2cnn(C)c2)n1 ; CN(C)CCc1ccc(O)cc1 ; Cc1cccc(C)c1-c1cc(Oc2ccc(CCN(C)C)cc2)nc(NS(=O)(=O)c2cnn(C)c2)n1]

To a solution of N-[4-chloro-6-(2,6-dimethylphenyl)pyrimidin-2-yl]-1-methyl-pyrazole-4-sulfonamide (26 mg, 0.06881 mmol) and 4-[2-(dimethylamino)ethyl]phenol (35 mg, 0.2118 mmol) in NMP (0.4 mL) was added Cs₂CO₃ (101 mg, 0.3100 mmol) and the reaction mixture was stirred at 120° C. for 16 hours. The reaction mixture was diluted with MeOH, filtered and purified by HPLC (1-99% ACN in water (HCl modifier)) to give N-[4-[4-[2-(dimethylamino)ethyl]phenoxy]-6-(2,6-dimethylphenyl)pyrimidin-2-yl]-1-methyl-pyrazole-4-sulfonamide (hydrochloride salt) (24.6 mg, 66%) as a light reddish orange solid. ¹H NMR (400 MHz, DMSO-d₆) δ 10.15 (s, 1H), 7.65 (s, 1H), 7.45 (d, J=8.4 Hz, 2H), 7.32 (d, J=8.5 Hz, 2H), 7.29-7.21 (m, 1H), 7.21-7.09 (m, 3H), 6.61 (s, 1H), 3.75 (s, 3H), 3.32 (s, 2H), 3.16-3.02 (m, 2H), 2.83 (d, J=5.0 Hz, 6H), 2.04 (s, 6H). ESI-MS m/z calc. 506.21002, found 507.3 (M+1)⁺; Retention time: 1.12 minutes; LC method A.

### Example 436: Preparation of Compound 1469

### Step 1: N-[4-(2,6-Dimethylphenyl)-6-(4-morpholinophenoxy)pyrimidin-2-yl]-1 -methyl-pyrazole-4-sulfonamide

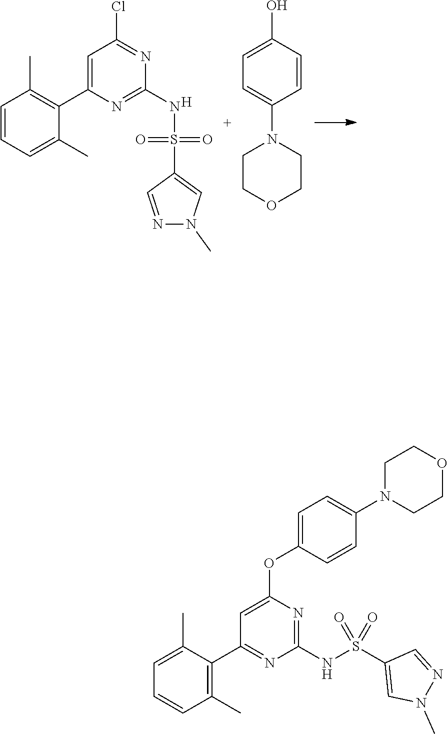

N-[4-chloro-6-(2,6-dimethylphenyl)pyrimidin-2-yl]-1-methyl-pyrazole-4-sulfonamide (19 g, 50.28 mmol) and 4-morpholinophenol (approximately 9.912 g, 55.31 mmol) were dissolved in NMP (190.0 mL), treated with K₂CO₃ (approximately 20.84 g, 150.8 mmol) and the suspension was heated in an oil bath at 125-130° C. for 40 hours. The brown reaction mixture was slowly added to a solution of AcOH (approximately 30.19 g, 28.59 mL, 502.8 mmol) in water (950.0 mL) and the formed suspension was stirred for 1 hour. The solid was collected by filtration. The wet solid was dissolved in DCM (475 mL) and washed with water (500 mL). The organic solution was dried over magnesium sulfate, treated with charcoal, and filtered. The DCM solution was treated with a PM-TMT Pd-scavenger resin (Biotage Part #801472, Lot #12170NJ) for 1 hour. The mixture was filtered, and the solvent evaporated. The crude (˜27 g) was suspended in DCM/methanol. Most of the DCM and methanol were removed under reduced pressure at 60° C. to give a thick suspension, which was stirred at room temperature for 1 hour and filtered. The solid was washed with dry ice cold methanol and dried in a drying cabinet at 45° C. with a nitrogen bleed over the weekend to give N-[4-(2,6-dimethylphenyl)-6-(4-morpholinophenoxy)pyrimidin-2-yl]-1-methyl-pyrazole-4-sulfonamide (21.4 g, 80%) as an off white solid. ¹H NMR (400 MHz, DMSO-d₆) δ 11.65 (s, 1H), 7.65 (s, 1H), 7.34 (s, 1H), 7.28-7.16 (m, 3H), 7.13 (d, J=7.6 Hz, 2H), 7.10-7.00 (m, 2H), 6.51 (s, 1H), 3.85-3.63 (m, 7H), 3.22-3.05 (m, 4H), 2.03 (s, 6H). ESI-MS m/z calc. 520.1893, found 521.0 (M+1)⁺; Retention time: 1.55 minutes; LC method A.

### Example 437: Preparation of Compound 1470

### Step 1: N-[4-(2,6-Dimethylphenyl)-6-[4-(4-hydroxyazepan-1-yl)phenoxy]pyrimidin-2-yl]-1-methyl-pyrazole-4-sulfonamide

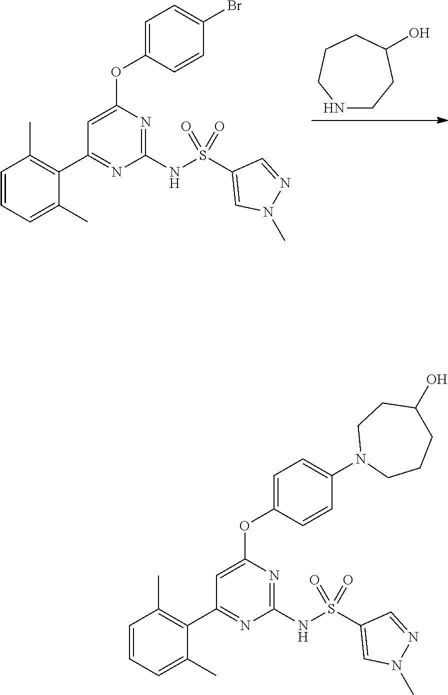 [SMILES: Cc1cccc(C)c1-c1cc(Oc2ccc(Br)cc2)nc(NS(=O)(=O)c2cnn(C)c2)n1 ; OC1CCCNCC1 ; Cc1cccc(C)c1-c1cc(Oc2ccc(N3CCCC(O)CC3)cc2)nc(NS(=O)(=O)c2cnn(C)c2)n1]

N-[4-(4-bromophenoxy)-6-(2,6-dimethylphenyl)pyrimidin-2-yl]-1-methyl-pyrazole-4-sulfonamide (20 mg, 0.03888 mmol), [2-(2-aminoethyl)phenyl]-chloro-palladium;ditert-butyl-[2-(2,4,6-triisopropylphenyl)phenyl]phosphane (XPhos Pd G1)(approximately 10.68 mg, 0.01555 mmol), azepan-4-ol (approximately 13.43 mg, 0.1166 mmol), sodium tert-butoxide (approximately 22.42 mg, 0.2333 mmol), and dioxane (0.8 mL) were combined in a glass vial and the mixture was sparged with nitrogen under sonication for 5 minutes. The reaction was stirred at room temperature for 15 minutes. AcOH (approximately 2.335 mg, 2.211 μL, 0.03888 mmol) was added to quench the reaction. The solution was filtered, and the resulting residue diluted in 0.8 mL MeOH and purified by reverse phase HPLC using a 15 minutes gradient of 20% MeCN in water to 80% MeCN to give N-[4-(2,6-dimethylphenyl)-6-[4-(4-hydroxyazepan-1-yl)phenoxy]pyrimidin-2-yl]-1-methyl-pyrazole-4-sulfonamide (6.4 mg, 30%). ESI-MS m z calc. 548.2206, found 549.63 (M+1)⁺; Retention time: 1.42 minutes; LC method A.

### Example 438: Preparation of Compound 1471

### Step 1: N-[4-(2,6-Dimethylphenyl)-6-[4-(1-methyl-4-piperidyl)phenoxy]pyrimidin-2-yl]-1-methyl-pyrazole-4-sulfonamide

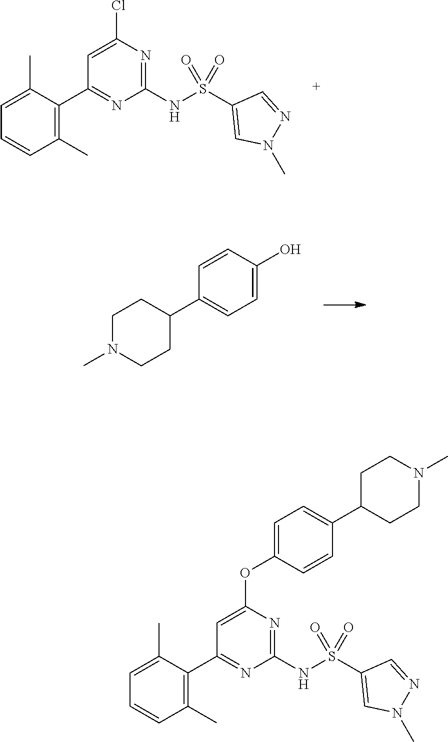

To a solution of N-[4-chloro-6-(2,6-dimethylphenyl)pyrimidin-2-yl]-1-methyl-pyrazole-4-sulfonamide (30 mg, 0.07940 mmol) and 4-(1-methyl-4-piperidyl)phenol acetate (approximately 59.86 mg, 0.2382 mmol) in NMP (0.4 mL) was added Cs₂CO₃ (approximately 103.5 mg, 0.3176 mmol) and the reaction mixture was stirred at 120° C. for 16 hours. The reaction mixture was diluted with MeOH, filtered and purified by HPLC (1-99% ACN in water (HCl modifier)) to give N-[4-(2,6-dimethylphenyl)-6-[4-(1-methyl-4-piperidyl)phenoxy]pyrimidin-2-yl]-1-methyl-pyrazole-4-sulfonamide (hydrochloride salt) (26.4 mg, 58%). ESI-MS m/z calc. 532.22565, found 533.5 (M+1)⁺; Retention time: 1.15 minutes (LC method A).

### Example 439: Preparation of Compound 1472

### Step 1: 1-(4-Hydroxyphenyl)piperidin-4-ol

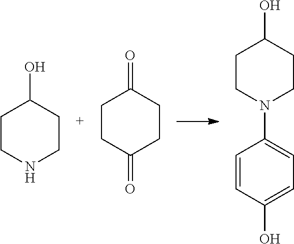

Piperidin-4-ol (200 mg, 1.977 mmol) and cyclohexane-1,4-dione (approximately 443.3 mg, 3.954 mmol) was refluxed in EtOH (9.885 mL) in open air for 16 hours and the reaction was then concentrated in vacuo and the resulting residue was purified by column chromatography (0 to 10% MeOH/DCM) to give clean fractions of 1-(4-hydroxyphenyl)piperidin-4-ol ESI-MS m/z calc. 193.11028, found 194.2 (M+1)⁺; Retention time: 0.1 minutes (LC method D).

### Step 2: N-[4-(2,6-Dimethylphenyl)-6-[4-(4-hydroxy-1-piperidyl)phenoxy]pyrimidin-2-yl]-1-methyl-pyrazole-4-sulfonamide

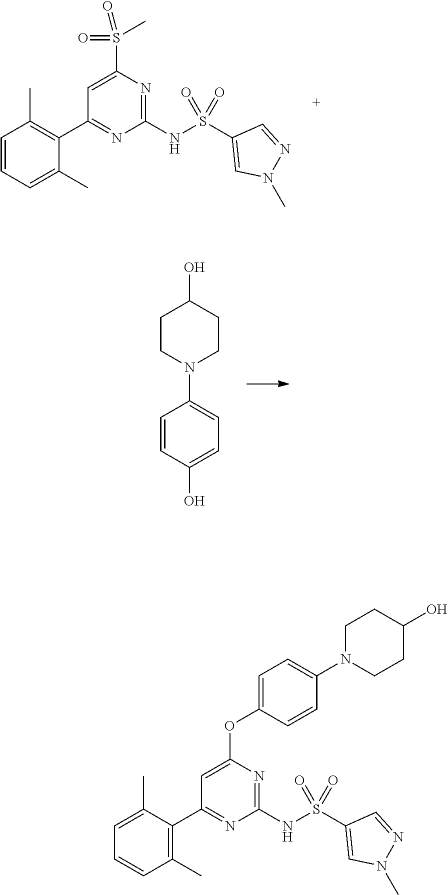 [SMILES: Cc1cccc(C)c1-c1cc(S(C)(=O)=O)nc(NS(=O)(=O)c2cnn(C)c2)n1 ; Oc1ccc(N2CCC(O)CC2)cc1 ; Cc1cccc(C)c1-c1cc(Oc2ccc(N3CCC(O)CC3)cc2)nc(NS(=O)(=O)c2cnn(C)c2)n1]

An NMP (600 μL) solution of N-[4-(2,6-dimethylphenyl)-6-methylsulfonyl-pyrimidin-2-yl]-1-methyl-pyrazole-4-sulfonamide (20 mg, 0.04745 mmol), cesium carbonate (61.7 mg, 0.1894 mmol), and 1-(4-hydroxyphenyl)piperidin-4-ol (24.9 mg, 0.1289 mmol) was heated at 110° C. for 1 hour and then diluted with ethyl acetate (1 mL) and water (0.5 mL). The pH of the aqueous layer was adjusted to 4 by the addition of concentrated HCl. After drying over sodium sulfate, the organic solution was concentrated. Purification by reverse phase HPLC gave N-[4-(2,6-dimethylphenyl)-6-[4-(4-hydroxy-1-piperidyl)phenoxy]pyrimidin-2-yl]-1 -methyl-pyrazole-4-sulfonamide (hydrochloride salt) (6.4 mg, 24%). ESI-MS m/z calc. 534.2049, found 535.48 (M+1)⁺; Retention time: 1.06 minutes; LC method A.

### Example 440: Preparation of Compound 1473

### Step 1: N-[4-(2,6-Dimethylphenyl)-6-[4-(1-methyl-2-piperidyl)phenoxy]pyrimidin-2-yl]-1-methyl-pyrazole-4-sulfonamide

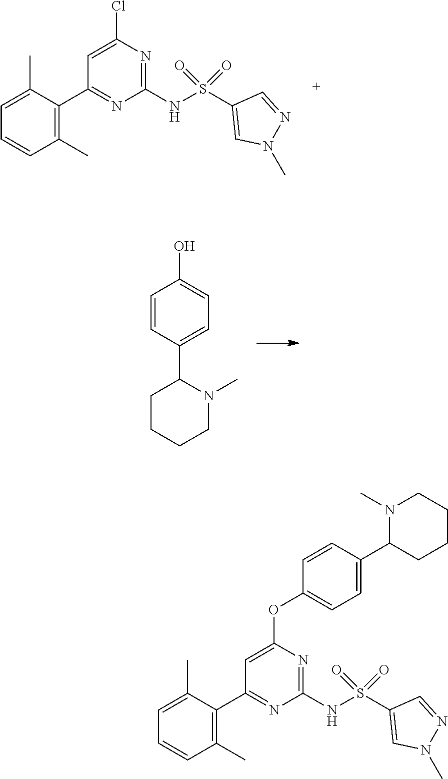 [SMILES: Cc1cccc(C)c1-c1cc(Cl)nc(NS(=O)(=O)c2cnn(C)c2)n1 ; CN1CCCCC1c1ccc(O)cc1 ; Cc1cccc(C)c1-c1cc(Oc2ccc(C3CCCCN3C)cc2)nc(NS(=O)(=O)c2cnn(C)c2)n1]

To a solution of N-[4-chloro-6-(2,6-dimethylphenyl)pyrimidin-2-yl]-1-methyl-pyrazole-4-sulfonamide (30 mg, 0.07940 mmol) and 4-(1-methyl-2-piperidyl)phenol (approximately 45.56 mg, 0.2382 mmol) in NMP (0.4 mL) was added Cs₂CO₃ (approximately 103.5 mg, 0.3176 mmol) and the reaction mixture was stirred at 120° C. for 16 hours. The mixture was diluted with MeOH, filtered and purified by HPLC (1-99% ACN in water (HCl modifier)) to give N-[4-(2,6-dimethylphenyl)-6-[4-(1-methyl-2-piperidyl)phenoxy]pyrimidin-2-yl]-1-methyl-pyrazole-4-sulfonamide (hydrochloride salt) (27.7 mg, 62%). ESI-MS m/z calc. 532.22565, found 533.3 (M+1)⁺; Retention time: 1.02 minutes; LC method A.

### Example 441: Preparation of Compound 1474

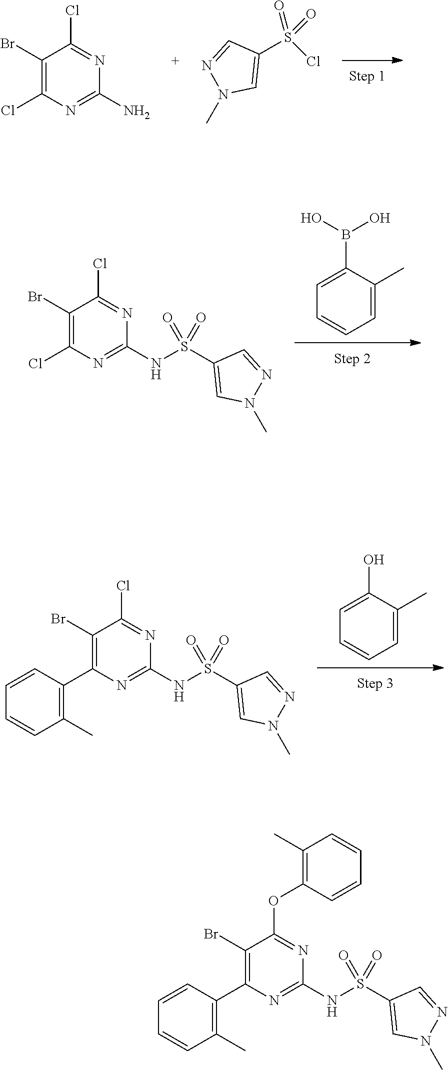

### Step 1: N-(5-Bromo-4,6-dichloro-pyrimidin-2-yl)-1-methyl-pyrazole-4-sulfonamide

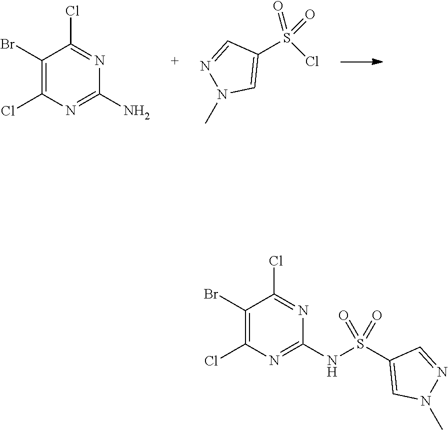

To a solution of 5-bromo-4,6-dichloro-pyrimidin-2-amine (3 g, 12.35 mmol) in DMF (50 mL) at 0° C. was added sodium hydride (1.98 g of 60% w/w, 49.50 mmol). The reaction was allowed to warm to 23° C. over 15 min and then cooled back to 0° C. before adding 1-methylpyrazole-4-sulfonyl chloride (4.46 g, 24.69 mmol). The reaction mixture was allowed again to warm to 23° C. over 15 minutes and then it was quenched using acetic acid (10.50 mL, 184.6 mmol). The solution was further diluted with water and ethyl acetate. The organic layer was separated, and the aqueous layer was extracted with ethyl acetate (4×). The combined organics were washed with brine, dried over magnesium sulfate, filtered and concentrated in vacuo. The crude residue was triturated with acetone (2×) to afford N-(5-bromo-4,6-dichloro-pyrimidin-2-yl)-1-methyl-pyrazole-4-sulfonamide (4.09 g, 80%) as a white solid. ESI-MS m z calc. 386.88, found 388.14 (M+1)⁺; Retention time: 0.51 minutes; LC method D.

### Step 2: N-[5-bromo-4-chloro-6-(o-tolyl)pyrimidin-2-yl]-1-methyl-pyrazole-4-sulfonamide

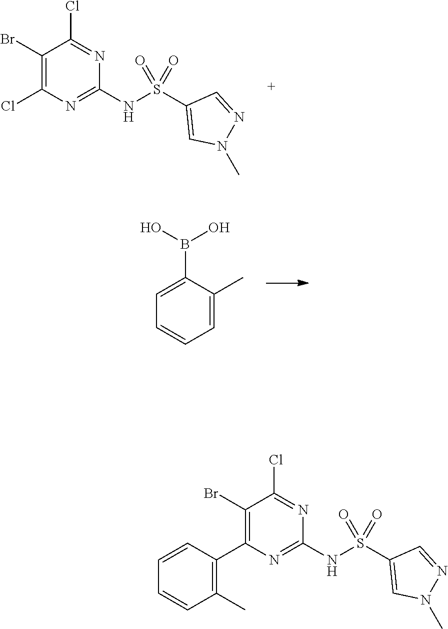

A heterogeneous solution consisting of N-(5-bromo-4,6-dichloro-pyrimidin-2-yl)-1 -methyl-pyrazole-4-sulfonamide (1.2 g, 2.883 mmol), tetrakis(triphenylphosphine)palladium(0) (666 mg, 0.5763 mmol), potassium carbonate (1.2 g, 8.683 mmol), and o-tolylboronic acid (392 mg, 2.883 mmol) in dioxane (12 mL) and water (2.4 mL) was microwaved in a sealed vial at 120° C. for 20 minutes. After cooling, the dioxane layer was removed under a steady stream of air. The remaining contents were acidified using acetic acid (2.5 mL, 43.96 mmol) and allowed to stir until bubbling ceased. Ethyl acetate was added and then the organic layer was removed. The aqueous layer was further extracted with ethyl acetate (2×). The combined organics were dried over magnesium sulfate, filtered, and concentrated in vacuo. The crude residue was dissolved in DMSO (2.0 mL) and filtered through a 0.20 μM PTFE syringe filter. The product was purified by preparatory HPLC (gradient: 10 to 99% acetonitrile in water with 0.1% hydrochloric acid) to afford N-[5-bromo-4-chloro-6-(o-tolyl)pyrimidin-2-yl]-1-methyl-pyrazole-4-sulfonamide (130 mg, 10%) as a white solid. ESI-MS m/z calc. 440.9662, found 444.27 (M+1)⁺; Retention time: 0.62 minutes; LC method D.

### Step 3: N-[5-bromo-4-(2-methylphenoxy)-6-(o-tolyl)pyrimidin-2-yl]-1-methyl-pyrazole-4-sulfonamide

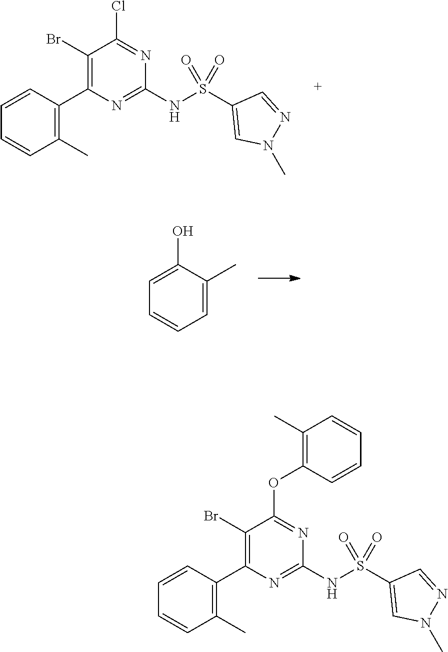

A heterogeneous mixture consisting of N-[5-bromo-4-chloro-6-(o-tolyl)pyrimidin-2-yl]-1-methyl-pyrazole-4-sulfonamide (120 mg, 0.2711 mmol), o-cresol (88 mg, 0.8138 mmol), and potassium carbonate (187 mg, 1.353 mmol) in NMP (550 μL) was heated to 110° C. in a sealed vial for 16 hours. Water (0.25 mL) and acetic acid (300 μL, 5.275 mmol) were added. The solution was allowed to stir until neutralized, DMSO (0.5 mL) was added and the solution was filtered. The solution was purified by preparative HPLC (gradient: 10% to 99% acetonitrile in water with 0.1% hydrochloric acid) to afford N-[5-bromo-4-(2-methylphenoxy)-6-(o-tolyl)pyrimidin-2-yl]-1-methyl-pyrazole-4-sulfonamide (3 mg, 2%) as a white solid. ¹H NMR (400 MHz, DMSO-d₆) δ 11.80 (s, 1H), 7.54-7.20 (m, 9H), 7.03 (d, J=0.7 Hz, 1H), 3.75 (s, 3H), 2.14 (d, J=5.1 Hz, 6H). ESI-MS m/z calc. 513.047, found 516.0 (M+1)⁺; Retention time: 0.72 minutes; LC method D.

### Example 442: Characterization of Compounds 580, and 1475-1479

The compounds in the following tables were prepared in a manner analogous to that described above using commercially available reagents and intermediates described herein.

| Compound | | LCMS Rt | calc. | | LCMS |
|---|---|---|---|---|---|
| number | Structure | (min) | mass | M + 1 | Method |
| 1475 | 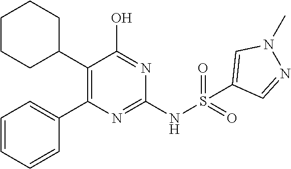 [SMILES: Cn1cc(S(=O)(=O)Nc2nc(O)c(C3CCCCC3)c(-c3ccccc3)n2)cn1] | 1.63 | 413.152 | 414.1 | A |
| 1476 | 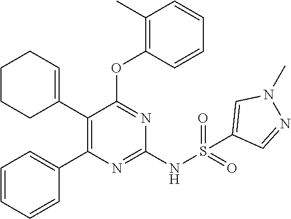 [SMILES: Cc1ccccc1Oc1nc(NS(=O)(=O)c2cnn(C)c2)nc(-c2ccccc2)c1C1=CCCCC1] | 2.13 | 501.183 | 502.2 | A |
| 580 | 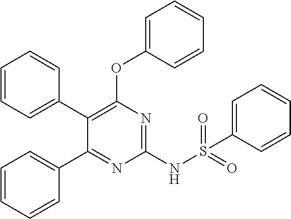 [SMILES: O=S(=O)(Nc1nc(Oc2ccccc2)c(-c2ccccc2)c(-c2ccccc2)n1)c1ccccc1] | 2.01 | 479.13 | 480.1 | A |
| 1477 | 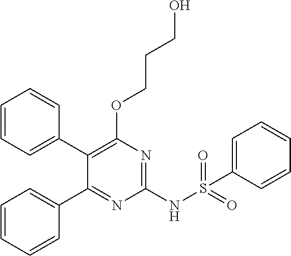 [SMILES: O=S(=O)(Nc1nc(OCCCO)c(-c2ccccc2)c(-c2ccccc2)n1)c1ccccc1] | 1.52 | 461.141 | 462.2 | A |
| 1478 | 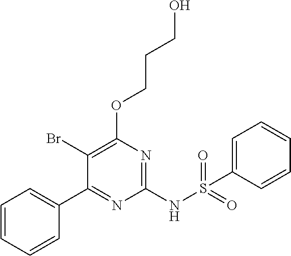 [SMILES: O=S(=O)(Nc1nc(OCCCO)c(Br)c(-c2ccccc2)n1)c1ccccc1] | 1.5 | 463.02 | 464 | A |
| 1479 | 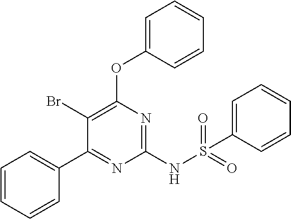 [SMILES: O=S(=O)(Nc1nc(Oc2ccccc2)c(Br)c(-c2ccccc2)n1)c1ccccc1] | 1.96 | 481.01 | 482.1 | A |

| Compound | |
|---|---|
| number | NMR |
| 1476 | ¹H NMR (400 MHz, DMSO-d₆) δ 11.50 (s, 1H), 7.74-7.57 (m, 2H), 7.50-7.42 |
| | (m, 4H), 7.42-7.38 (m, 1H), 7.38-7.30 (m, 1H), 7.30-7.24 (m, 1H), 7.21-7.10 |
| | (m, 2H), 5.64-5.51 (m, 1H), 3.74 (s, 3H), 2.20-2.14 (m, 2H), 2.13 (s, 3H), 2.02- |
| | 1.87 (m, 2H), 1.66-1.42 (m, 4H). |
| 1477 | ¹H NMR (400 MHz, DMSO-d₆) δ 8.02-7.94 (m, 2H), 7.70-7.63 (m, 1H), 7.62- |
| | 7.55 (m, 2H), 7.31-7.24 (m, 1H), 7.24-7.17 (m, 5H), 7.17-7.11 (m, 2H), 7.09- |
| | 7.01 (m, 2H), 4.18 (t, J = 6.3 Hz, 2H), 3.36 (t, J = 6.2 Hz, 2H), 1.71-1.60 (m, |
| | 2H). |
| 1478 | ¹H NMR (400 MHz, DMSO-d₆) δ 12.00 (s, 1H), 8.01-7.84 (m, 2H), 7.72-7.65 |
| | (m, 1H), 7.63-7.55 (m, 2H), 7.54-7.44 (m, 5H), 4.29 (t, J = 6.4 Hz, 2H), 3.53 |
| | (t, J = 6.2 Hz, 2H), 1.83 (p, J = 6.3 Hz, 2H). |
| 1479 | ¹H NMR (400 MHz, DMSO-d₆) δ 11.92 (s, 1H), 7.62-7.55 (m, 5H), 7.54-7.48 |
| | (m, 3H), 7.45-7.35 (m, 5H), 7.29-7.23 (m, 2H). |

### Example 443: Preparation of Compound 1480

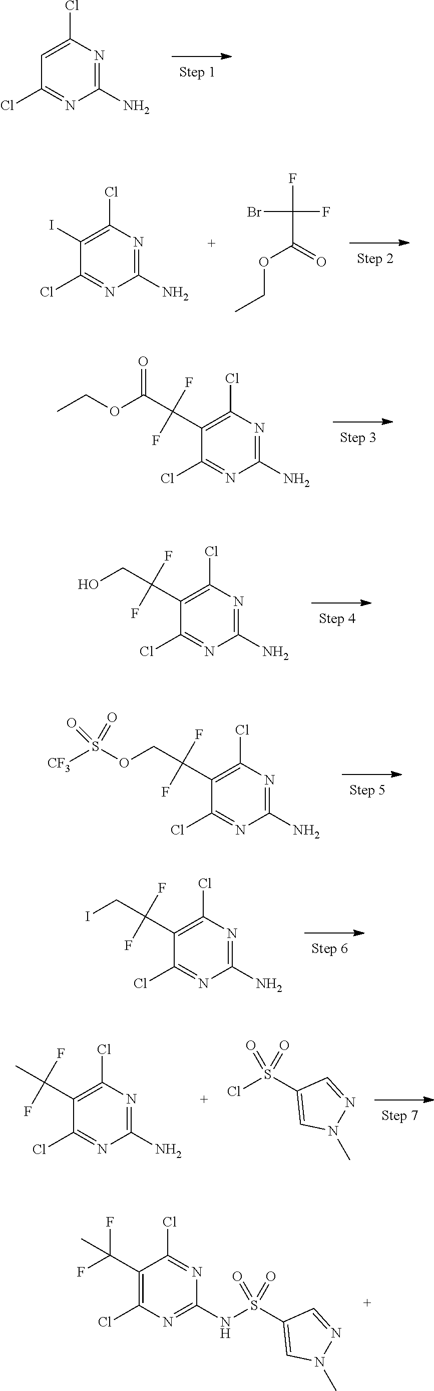

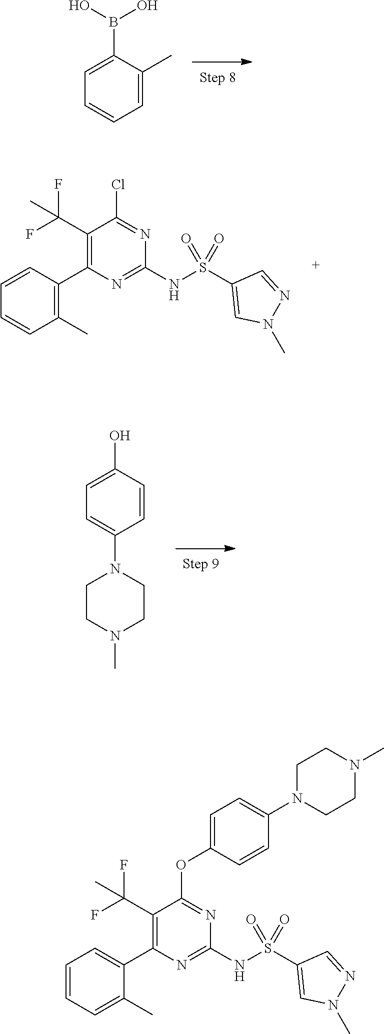

### Step 1: 4,6-Dichloro-5-iodo-pyrimidin-2-amine

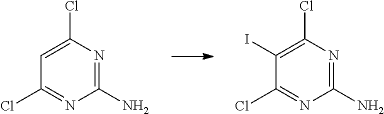 [SMILES: Nc1nc(Cl)cc(Cl)n1 ; Nc1nc(Cl)c(I)c(Cl)n1]

Iodine monochloride (45 g, 277.17 mmol) was dissolved in acetic acid (300 mL) at room temperature and then 4,6-dichloropyrimidin-2-amine (15 g, 91.467 mmol) was added slowly to the solution. The reaction was stirred overnight at room temperature. The precipitate was filtered. The solid residue was washed with acetic acid (50 mL) and dried under high vacuum. The acetic acid salt was dissolved in ethyl acetate (150 mL) and added to a stirring saturated sodium bicarbonate aqueous solution (250 mL). The mixture was stirred for 20 minutes then the layers were separated. The aqueous layer was extracted with ethyl acetate (150 mL) and the combined organic layers were dried over sodium sulfate, filtered and concentrated under reduced pressure to give 4,6-dichloro-5-iodo-pyrimidin-2-amine (12 g, 45%) as an off-white solid; ¹H NMR (300 MHz, DMSO-d₆) δ 7.63 (br. s., 2H). ESI-MS m/z calc. 288.86703, found 289.9 (M+1)⁺; Retention time: 1.79 minutes (LC method C).

### Step 2: Ethyl 2-(2-amino-4,6-dichloro-pyrimidin-5-yl)-2,2-difluoro-acetate

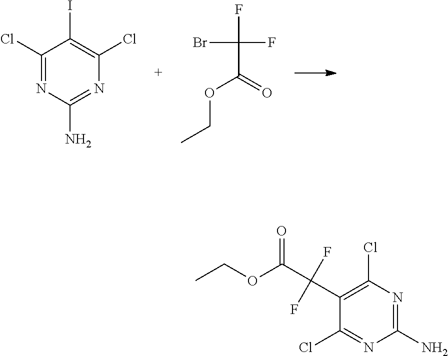

A solution of 4,6-dichloro-5-iodo-pyrimidin-2-amine (250 mg, 0.8624 mmol), ethyl 2-bromo-2,2-difluoro-acetate (182.05 mg, 0.115 mL, 0.8969 mmol) and copper (110 mg, 1.7310 mmol) in dimethylsulfoxide (2.5 mL) was degassed for 10 min then sealed and heated to 40° C. for 16 hours. A saturated ammonium chloride aqueous solution (10 mL) was added and the mixture was extracted with ethyl acetate (2×15 mL). The combined organic layers were adsorbed on silica gel and concentrated under reduced pressure. The crude residue was purified by flash chromatography using a 12 g silica column eluting from 5% to 25% ethyl acetate in heptanes to give ethyl 2-(2-amino-4,6-dichloro-pyrimidin-5-yl)-2,2-difluoro-acetate (111 mg, 45%) as a white solid. ¹H NMR (CDCl₃, 300 MHz) ppm δ 1.36 (t, J=7.2 Hz, 3H), 4.39 (q, J=7.2 Hz, 2H), 5.57 (br. s, 2H). ¹⁹F NMR (CDCl₃, 282 MHz) δ ppm −96.7 (s, 2 F).

### Step 3: 2-(2-Amino-4,6-dichloro-pyrimidin-5-yl)-2,2-difluoro-ethanol

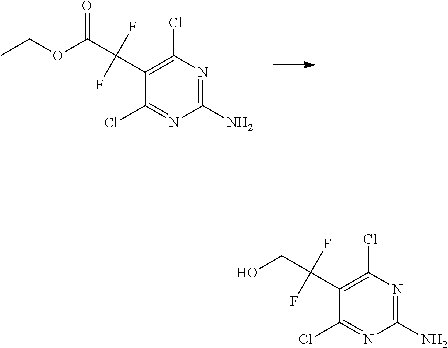 [SMILES: CCOC(=O)C(F)(F)c1c(Cl)nc(N)nc1Cl ; Nc1nc(Cl)c(C(F)(F)CO)c(Cl)n1]

To a solution of ethyl 2-(2-amino-4,6-dichloro-pyrimidin-5-yl)-2,2-difluoro-acetate (543 mg, 1.8982 mmol) in ethanol (18 mL) at 0° C. was added calcium chloride (73 mg, 0.6578 mmol) followed by sodium borohydride (73 mg, 1.9296 mmol). The reaction was stirred for 0.5 hours at 0° C. then 3 hours at room temperature. A white suspension was observed. The reaction was cooled down to 0° C. and quenched by adding a saturated ammonium chloride solution (50 mL) and extracted with dichloromethane (2×60 mL). The combined organic layers were adsorbed on silica gel and concentrated under reduced pressure. The crude residue was purified by flash chromatography using a 24 g silica column eluting from 20% to 50% ethyl acetate in heptanes to give 2-(2-amino-4,6-dichloro-pyrimidin-5-yl)-2,2-difluoro-ethanol (317 mg, 68%) as a white solid. ¹H NMR (DMSO-d₆, 300 MHz) δ ppm 3.88 (td, J=14.1, 6.9 Hz, 2H), 5.70 (t, J=6.9 Hz, 1H), 7.90 (br. s, 2H). ¹⁹F NMR (DMSO-d₆, 282 MHz) δ ppm −97.0 (t, J=14.3 Hz, 2 F).

### Step 4: [2-(2-Amino-4,6-dichloro-pyrimidin-5-yl)-2,2-difluoro-ethyl]trifluoromethanesulfonate

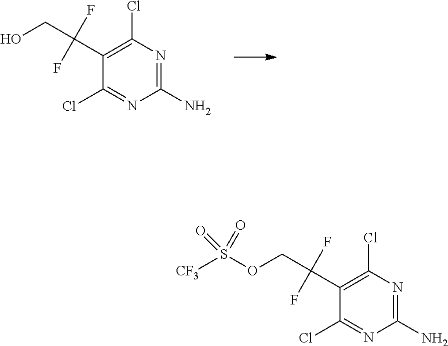 [SMILES: Nc1nc(Cl)c(C(F)(F)CO)c(Cl)n1 ; Nc1nc(Cl)c(C(F)(F)COS(=O)(=O)C(F)(F)F)c(Cl)n1]

To solution of 2-(2-amino-4,6-dichloro-pyrimidin-5-yl)-2,2-difluoro-ethanol (25 mg, 0.1024 mmol) in diethyl ether (2 mL) at 0° C. were added DIPEA (37.100 mg, 0.050 mL, 0.2871 mmol) followed by trifluoromethanesulfonic anhydride (58.695 mg, 0.035 mL, 0.2080 mmol). The reaction was warmed up to room temperature and stirred for 2 hours. The reaction was filtered over Celite and the solid residue washed with diethyl ether. The filtrate was concentrated under reduced pressure to give crude [2-(2-amino-4,6-dichloro-pyrimidin-5-yl)-2,2-difluoro-ethyl] trifluoromethanesulfonate (53 mg, quantitative) as a brown oil. The crude product was used to the next step without any purification. ESI-MS m/z calc. 374.92706, found 376.0 (M+1)⁺; Retention time: 2.07 minutes; LC method C.

### Step 5: 4,6-Dichloro-5-(1,1-difluoro-2-iodo-ethyl)pyrimidin-2-amine

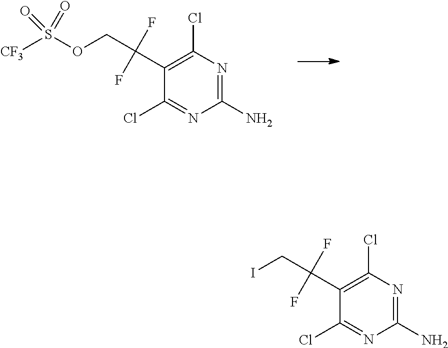 [SMILES: Nc1nc(Cl)c(C(F)(F)COS(=O)(=O)C(F)(F)F)c(Cl)n1 ; Nc1nc(Cl)c(C(F)(F)CI)c(Cl)n1]

To a solution of crude [2-(2-amino-4,6-dichloro-pyrimidin-5-yl)-2,2-difluoro-ethyl]trifluoromethanesulfonate (369.88 mg, 0.9835 mmol) in acetonitrile (15 mL) was added sodium iodide (443 mg, 2.9554 mmol) and the resulting mixture was stirred at 70° C. for 0.5 hours. The reaction was cooled down to room temperature, adsorbed on silica gel and concentrated under reduced pressure. The crude residue was purified by flash chromatography using a 24 g silica column eluting from 0% to 20% ethyl acetate in heptanes to give 4,6-dichloro-5-(1,1-difluoro-2-iodo-ethyl)pyrimidin-2-amine (245 mg, 58%) as an orange solid. ¹H NMR (DMSO-d₆, 300 MHz) δ ppm 3.93 (t, J=16.8 Hz, 2H), 8.02 (br. s, 2H). ¹⁹F NMR (DMSO-d₆, 282 MHz) δ ppm −83.2 (t, J=16.9 Hz, 2 F). ESI-MS m/z calc. 352.87952, found 353.9 (M+1)⁺; Retention time: 1.95 minutes; LC method C.

### Step 6:4,6-Dichloro-5-(1,1-difluoroethyl)pyrimidin-2-amine

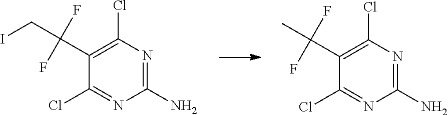

To a suspension of 4,6-dichloro-5-(1,1-difluoro-2-iodo-ethyl)pyrimidin-2-amine (455 mg, 1.2856 mmol) in toluene (25 mL) was added tributyltin hydride (757.40 mg, 0.70 mL, 2.6022 mmol) followed by 2,2′-azobis(2-methylpropionitrile) (33 mg, 0.2010 mmol). The mixture was degassed with nitrogen for 20 minutes, sealed and heated to 80° C. for 24 hours. The reaction was cooled down to room temperature. A 10% potassium fluoride aqueous solution (60 mL) and ethyl acetate (125 mL) were added and the mixture was filtered over Celite while washing with ethyl acetate. The phases were separated, the aqueous layer was back extracted with ethyl acetate (125 mL) and the combined organic layers were adsorbed on silica gel and concentrated under reduced pressure. The crude residue was purified by flash chromatography using a 80 g silica column eluting from 0% to 15% ethyl acetate in heptanes to give recovered starting material (209 mg, 43%) and desired 4,6-dichloro-5-(1,1-difluoroethyl)pyrimidin-2-amine (95 mg, 32%) as a white solid. ¹H NMR (DMSO-d₆, 300 MHz) δ ppm 1.98 (t, J=18.3 Hz, 3H), 7.88 (br. s, 2H). ¹⁹F NMR (DMSO-d₆, 282 MHz) δ ppm −79.0 (q, J=17.8 Hz, 2 F).

### Step 7: N-[4,6-Dichloro-5-(1,1-difluoroethyl)pyrimidin-2-yl]-1-methyl-pyrazole-4-sulfonamide

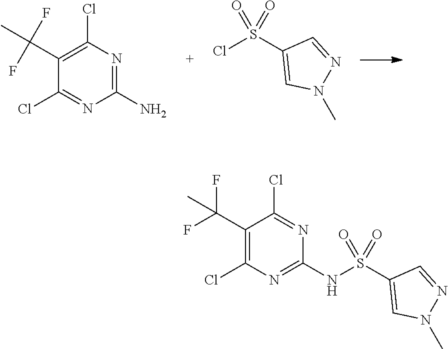

A DMF (2 mL) suspension of NaH (59 mg, 2.459 mmol) was cooled to 0° C. using an ice-water bath and was treated with solid 4,6-dichloro-5-(1,1-difluoroethyl)pyrimidin-2-amine (155 mg, 0.6797 mmol). The reaction mixture was stirred for 20 minutes and then treated with solid 1-methylpyrazole-4-sulfonyl chloride (125 mg, 0.6921 mmol). The reaction mixture was warmed to room temperature and stirred for 1 hour and then cooled to 0° C. HCl (1 mL of 1 M, 1.000 mmol) was added and the reaction mixture was stirred at 0° C. for 10 minutes upon which the product crystallized out. The reaction mixture was filtered, rinsed with cold water, and dried overnight under vacuum. The crude was purified by reverse phase preparative chromatography using a C₁₈ column and a 15 minutes gradient eluent of 10 to 60% acetonitrile in water containing 5 mM hydrochloric acid to give N-[4,6-dichloro-5-(1,1-difluoroethyl)pyrimidin-2-yl]-1-methyl-pyrazole-4-sulfonamide (96.1 mg, 37%). ¹H NMR (400 MHz, DMSO-d₆) δ 12.55 (s, 1H), 8.42 (s, 1H), 7.93 (s, 1H), 3.88 (s, 3H), 2.10-1.96 (m, 3H). ESI-MS m/z calc. 370.9822, found 372.0 (M+1)⁺; Retention time: 1.31 minutes; LC method A.

### Step 8: N-[4-Chloro-5-(1,1-difluoroethyl)-6-(o-tolyl)pyrimidin-2-yl]-1-methyl-pyrazole-4-sulfonamide

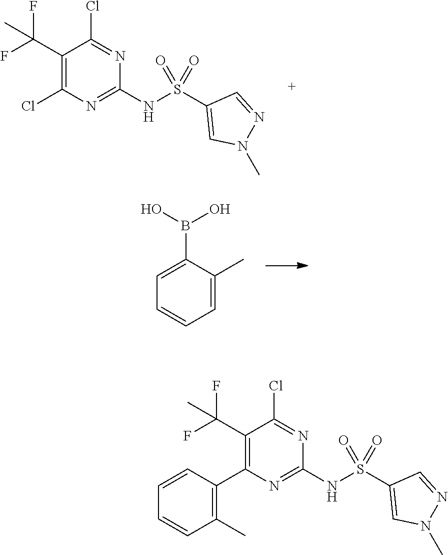

N-[4,6-dichloro-5-(1,1-difluoroethyl)pyrimidin-2-yl]-1-methyl-pyrazole-4-sulfonamide (96 mg, 0.2502 mmol), o-tolylboronic acid (36 mg, 0.2648 mmol), tetrakis(triphenylphosphine)palladium (0) (15 mg, 0.01298 mmol), and 2 M sodium carbonate (450 μL of 2 M, 0.9000 mmol) in 1,2-dimethoxyethane (2 mL) were combined in a microwave vial. The mixture was sparged with nitrogen, sealed and stirred for 65 hours at 70° C. The reaction mixture was diluted with water, extracted with ethyl acetate. The organic layer was dried over sodium sulfate, concentrated, and purified on reverse phase HPLC (HCl modifier, 25-75% ACN-H₂O) to give N-[4-chloro-5-(1,1-difluoroethyl)-6-(o-tolyl)pyrimidin-2-yl]-1-methyl-pyrazole-4-sulfonamide (58.3 mg, 54%). ¹H NMR (400 MHz, DMSO-d₆) δ 8.21 (s, 1H), 7.75 (s, 1H), 7.38-7.29 (m, 2H), 7.27-7.21 (m, 1H), 7.16 (d, J=7.7 Hz, 1H), 3.81 (s, 3H), 2.03 (s, 3H), 1.94 (t, J=18.7 Hz, 3H). ESI-MS m/z calc. 427.0681, found 428.0 (M+1)⁺; Retention time: 1.53 minutes; LC method A.

### Step 9: N-[5-(1,1-Difluoroethyl)-4-[4-(4-methylpiperazin-1-yl)phenoxy]-6-(o-tolyl)pyrimidin-2-yl]-1-methyl-pyrazole-4-sulfonamide

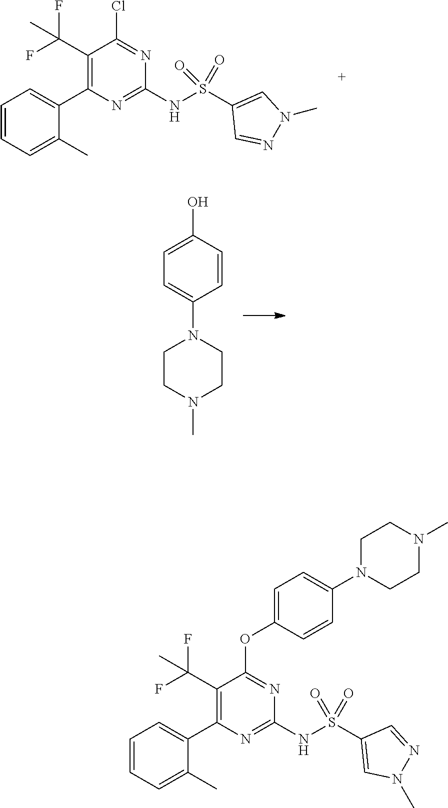 [SMILES: Cc1ccccc1-c1nc(NS(=O)(=O)c2cnn(C)c2)nc(Cl)c1C(C)(F)F ; CN1CCN(c2ccc(O)cc2)CC1 ; Cc1ccccc1-c1nc(NS(=O)(=O)c2cnn(C)c2)nc(Oc2ccc(N3CCN(C)CC3)cc2)c1C(C)(F)F]

An NMP (300 μL) mixture of N-[4-chloro-5-(1,1-difluoroethyl)-6-(o-tolyl)pyrimidin-2-yl]-1-methyl-pyrazole-4-sulfonamide (25 mg, 0.05843 mmol), 4-(4-methylpiperazin-1-yl)phenol (16 mg, 0.08322 mmol), and cesium carbonate (76 mg, 0.2333 mmol) was stirred at 110° C. for 16 hours. The reaction mixture was filtered and purified by reverse phase chromatography using a 15 minutes gradient of 10% MeCN in water to 60% MeCN (HCl modifier) to give N-[5-(1,1-difluoroethyl)-4-[4-(4-methylpiperazin-1-yl)phenoxy]-6-(o-tolyl)pyrimidin-2-yl]-1-methyl-pyrazole-4-sulfonamide (23 mg, 67%). ¹H NMR (400 MHz, DMSO-d₆) δ 10.90 (s, 1H), 7.46 (s, 1H), 7.29 (dd, J=12.8, 8.6 Hz, 5H), 7.20-7.14 (m, 3H), 7.11 (s, 1H), 3.85 (s, 3H), 3.73 (s, 3H), 3.51 (d, J=10.1 Hz, 2H), 3.16 (t, J=12.3 Hz, 4H), 2.81 (d, J=4.6 Hz, 3H), 2.10 (s, 3H), 1.98 (t, J=18.9 Hz, 3H). ESI-MS m/z calc. 583.2177, found 584.0 (M+1)⁺; Retention time: 1.25 minutes; LC method A.

### Example 444: Preparation of Compound 1481

### Step 1: N-[5-(1,1-Difluoroethyl)-4-[4-(1-methyl-4-piperidyl)phenoxy]-6-(o-tolyl)pyrimidin-2-yl]-1-methyl-pyrazole-4-sulfonamide

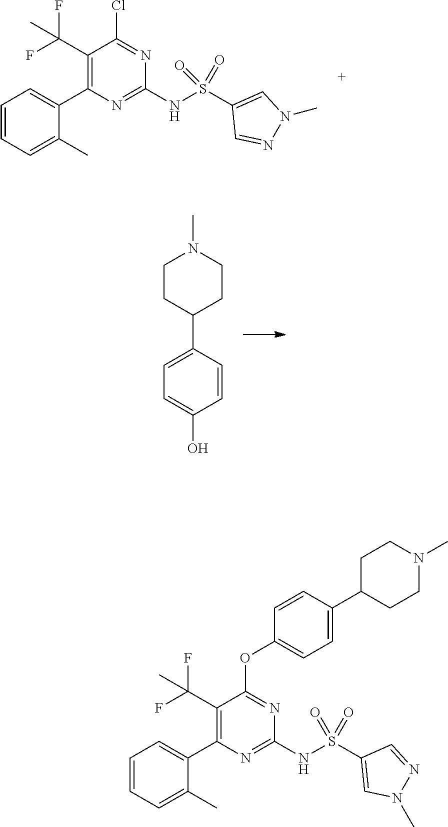 [SMILES: Cc1ccccc1-c1nc(NS(=O)(=O)c2cnn(C)c2)nc(Cl)c1C(C)(F)F ; CN1CCC(c2ccc(O)cc2)CC1 ; Cc1ccccc1-c1nc(NS(=O)(=O)c2cnn(C)c2)nc(Oc2ccc(C3CCN(C)CC3)cc2)c1C(C)(F)F]

An NMP (300 μL) mixture of N-[4-chloro-5-(1,1-difluoroethyl)-6-(o-tolyl)pyrimidin-2-yl]-1-methyl-pyrazole-4-sulfonamide (25 mg, 0.05843 mmol), 4-(1-methyl-4-piperidyl)phenol (16 mg, 0.08365 mmol), and cesium carbonate (76 mg, 0.2333 mmol) was stirred at 110° C. for 16 hours. The reaction mixture was cooled down to room temperature, filtered, and purified by reverse phase preparative chromatography using a C₁₈ column and a 15 minutes. gradient eluent of 10 to 60% acetonitrile in water containing 5 mM hydrochloric acid to give N-[5-(1,1-difluoroethyl)-4-[4-(1-methyl-4-piperidyl)phenoxy]-6-(o-tolyl)pyrimidin-2-yl]-1-methyl-pyrazole-4-sulfonamide (19.6 mg, 57%). ¹H NMR (400 MHz, DMSO-d₆) δ 10.39 (s, 1H), 7.47-7.39 (m, 3H), 7.35-7.28 (m, 4H), 7.26-7.20 (m, 1H), 7.15 (d, J=7.4 Hz, 1H), 6.95 (s, 1H), 3.73 (s, 3H), 3.53-3.43 (m, 4H), 3.14-3.01 (m, 2H), 2.99-2.83 (m, 2H), 2.78 (d, J=4.8 Hz, 3H), 2.10 (s, 3H), 2.06-2.00 (m, 4H). ESI-MS m/z calc. 582.2225, found 583.0 (M+1)⁺; Retention time: 1.26 minutes; LC method A.

### Example 445: Preparation of Compound 1482

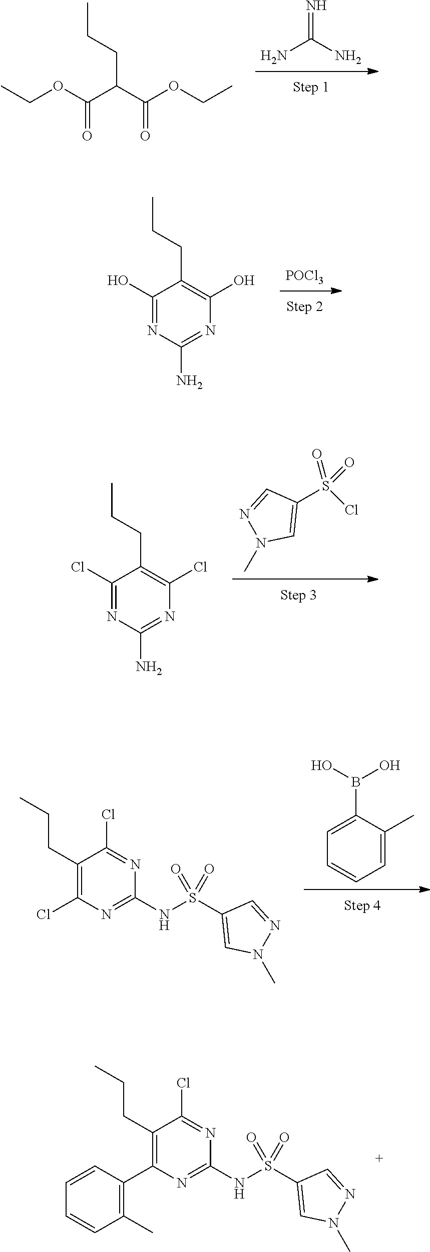

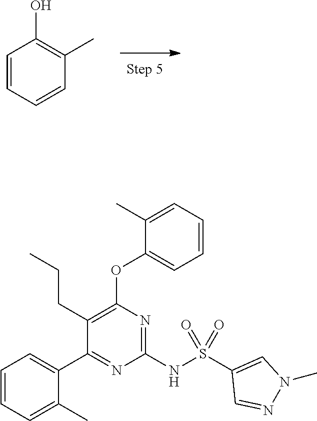

Step 1:2-Amino-5-propyl-pyrimidibe-4,6-diol

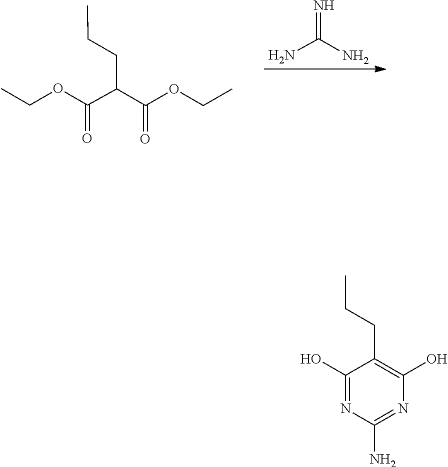

To a 1.0-L flask was added ethanol (400 mL). Solid chunks of sodium metal (9.91 g, 431 mmol) were added gradually and carefully and the mixture was stirred until sodium was completely dissolved. Once cooled back to room temperature guanidine hydrochloride (15.76 g, 165 mmol) and diethyl 2-propylpropanedioate (26.13 g, 129.2 mmol) were successively added and the reaction was stirred in an oil bath set at 80° C. for 17 hours. Once cooled down to room temperature, the crude mixture was concentrated under reduced pressure to remove most of the ethanol. Water (200 mL) was added, the resulting solution was cooled in an ice bath and acidified to pH of 1-2 using concentrated HCl. The solids were filtered and washed with water (2×50 mL). The solid was washed with acetone (about 250 mL) and dried under high vacuum to provide 2-amino-5-propyl-pyrimidine-4,6-diol hydrate (19.15 g, 79% yield) as a white solid.

¹H NMR (300 MHz, DMSO-d₆) δ ppm 0.81 (t, J=7.4 Hz, 3H), 1.23-1.40 (m, 2H), 2.10 (t, J=7.4Hz, 2H), 6.27 (s, 2H), 10.2 (br. s, 2H). ESI-MS m/z calc. 169.08513, found 170.2 (M+1)⁺; Retention time: 0.47 minutes; LC method C.

### Step 2: 4,6-Dichloro-5-propyl-pyrimidin-2-amine

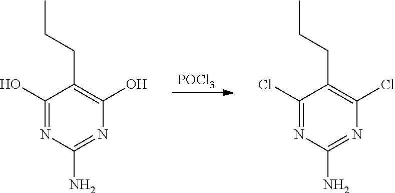

A suspension of 2-amino-5-propyl-pyrimidine-4,6-diol hydrate (19.15 g, 102.3 mmol) in dioxane (400 mL) and phosphoryl trichloride (48 mL, 515.0 mmol) was gradually heated up from room temperature to 100° C. in an oil bath and left to stir at that temperature for 45 hours. Once cooled, the dark crude mixture was concentrated under reduced pressure and co-evaporated with additional dioxane (1×200 mL and 1×150 mL). The crude residue was taken up in THF (250 mL) and 3 N aqueous HCl (250 mL) was added, maintaining the internal temperature below 40° C. with the use of a water bath. The mixture was then heated in an oil bath set at 60° C. for 6 hours. Once cooled to room temperature the crude mixture was transferred to a 2.0-L separatory funnel, diluted with brine (200 mL) and extracted with ethyl acetate (3×300 mL). The combined organic layers were washed with water:brine (1:1, 400 mL), brine (300 mL), dried over sodium sulfate, filtered and concentrated under reduced pressure to remove most of the ethyl acetate. Some solids crashed out and were filtered, washed with ethyl acetate and dried under high vacuum to afford a first lot of 4,6-dichloro-5-propyl-pyrimidin-2-amine (3.459 g, 16% yield) as an off-white solid (Lot 1). The filtrate was concentrated under reduced pressure and the residue was purified by silica gel chromatography on two separate 220-g columns, eluting from 0% to 10% ethyl acetate in dichloromethane. The cleanest fractions containing product were concentrated under reduced pressure to remove most of the dichloromethane, leaving a white solid suspended in ethyl acetate. This solid was filtered, washed with ethyl acetate and dried under high vacuum to afford a second lot of 4,6-dichloro-5-propyl-pyrimidin-2-amine (2.80 g, 13% yield) as a white solid (Lot 2). ¹H NMR (300 MHz, DMSO-d₆) δ ppm 0.92 (t, J=7.2 Hz, 3H), 1.41-1.61 (m, 2H), 2.53-2.66 (m, 2H), 7.33 (s, 2H). ESI-MS m/z calc. 205.01735, found 206.1 (M+1)⁺; Retention time: 2.78 minutes; LC method H.

### Step 3: N-(4,6-Dichloro-5-propyl-pyrimidin-2-yl)-1-methyl-pyrazole-4-sulfonamide

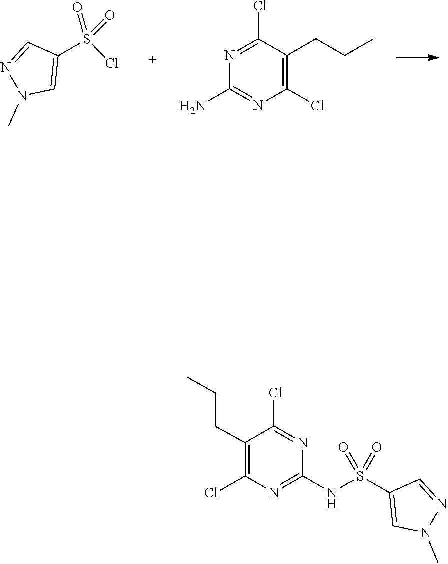

To a solution of 4,6-dichloro-5-propyl-pyrimidin-2-amine (511 mg, 2.480 mmol) in DMF (10.0 mL) at 0° C. was added sodium hydride (397 mg of 60% w/w, 9.926 mmol). The reaction was allowed to warm to 23° C. over 15 minutes and then cooled back to 0° C. before adding 1-methylpyrazole-4-sulfonyl chloride (896 mg, 4.961 mmol). The reaction mixture was allowed again to warm to 23° C. over 15 minutes and then it was quenched using acetic acid (2.1 mL, 36.93 mmol). The solution was further diluted with water and ethyl acetate. The organic layer was separated, and the aqueous layer was extracted using ethyl acetate (4×). The combined organics were washed with brine, dried over magnesium sulfate, filtered and concentrated in vacuo. The crude residue was purified by flash column chromatography on silica gel (gradient: 1 to 6% methanol in dichloromethane) to afford N-(4,6-dichloro-5-propyl-pyrimidin-2-yl)-1 -methyl-pyrazole-4-sulfonamide (780 mg, 29%) as a white solid. ESI-MS m/z calc. 349.0167, found 383.9 (M+1)⁺; Retention time: 0.58 minutes; LC method D.

### Step 4: N-[4-chloro-6-(o-tolyl)-5-propyl-pyrimidin-2-yl]-1-methyl-pyrazole-4-sulfonamide

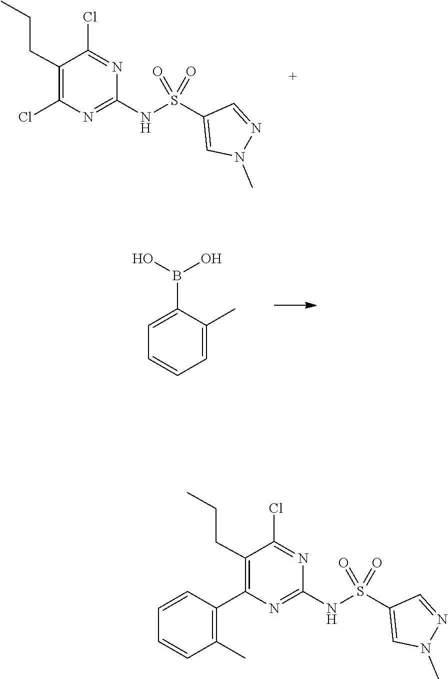

A heterogeneous solution of bis(triphenylphosphine)palladium(II) dichloride (approximately 11.81 mg, 0.01683 mmol), o-tolylboronic acid (approximately 76.27 mg, 0.5610 mmol), N-(4,6-dichloro-5-propyl-pyrimidin-2-yl)-1-methyl-pyrazole-4-sulfonamide (344.7 mg, 0.561 mmol) and potassium carbonate (approximately 232.6 mg, 1.683 mmol) in dioxane (1.870 mL) and water (374.0 μL) was heated in a sealed vial to 90° C. for 16 hours. The solution was acidified with acetic acid (approximately 33.69 mg, 31.90 μL, 0.5610 mmol), diluted with DMSO (1.0 mL), and filtered through a 0.45 m PTFE syringe filter. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[4-chloro-6-(o-tolyl)-5-propyl-pyrimidin-2-yl]-1-methyl-pyrazole-4-sulfonamide (30 mg, 12%) as a white solid. ESI-MS m/z calc. 405.10263, found 406.17 (M+1)⁺; Retention time: 0.68 minutes; LC method D.

### Step 5: 1-methyl-N-[4-(2-methylphenoxy)-6-(o-tolyl)-5-propyl-pyrimidin-2-yl]pyrazole-4-sulfonamide

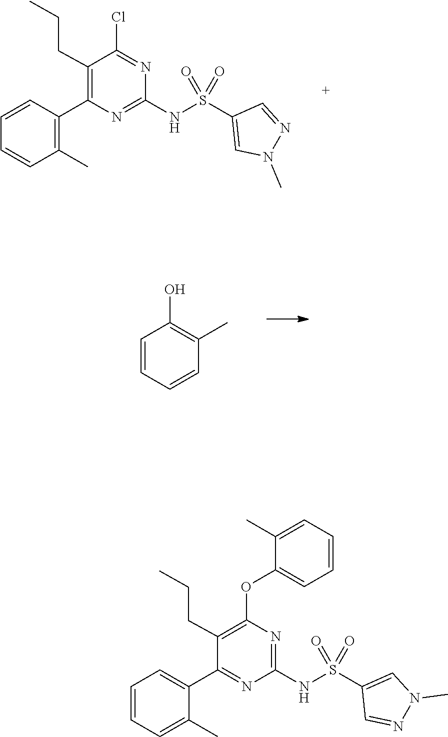

A heterogeneous solution consisting of o-cresol (12.98 mg, 23.47 μL, 0.1200 mmol), N-[4-chloro-6-(o-tolyl)-5-propyl-pyrimidin-2-yl]-1-methyl-pyrazole-4-sulfonamide (16.24 mg, 0.0400 mmol), and potassium carbonate (approximately 1.842 mg, 0.01333 mmol) in NMP was heated in a sealed vial to 110° C. for 16 hours. The solution was acidified using acetic acid(approximately 6.005 mg, 5.687 μL, 0.1000 mmol), diluted with DMSO (0.5 mL), and filtered using a 0.2 μM PTFE syringe filter. The crude solution was purified by preparative HPLC (gradient: 10 to 99% acetonitrile in water with 0.1% hydrochloric acid) to give 1-methyl-N-[4-(2-methylphenoxy)-6-(o-tolyl)-5-propyl-pyrimidin-2-yl]pyrazole-4-sulfonamide (7.8 mg, 41%) as a white solid. ESI-MS m/z calc. 477.18347, found 478.2 (M+1)⁺; Retention time: 1.92 minutes; LC method A.

### Example 446: Preparation of Compound 1483

### Step 1: 1-Methyl-N-[4-[4-(1-methyl-4-piperidyl)phenoxy]-6-(o-tolyl)-5-propyl-pyrimidin-2-yl]pyrazole-4-sulfonamide

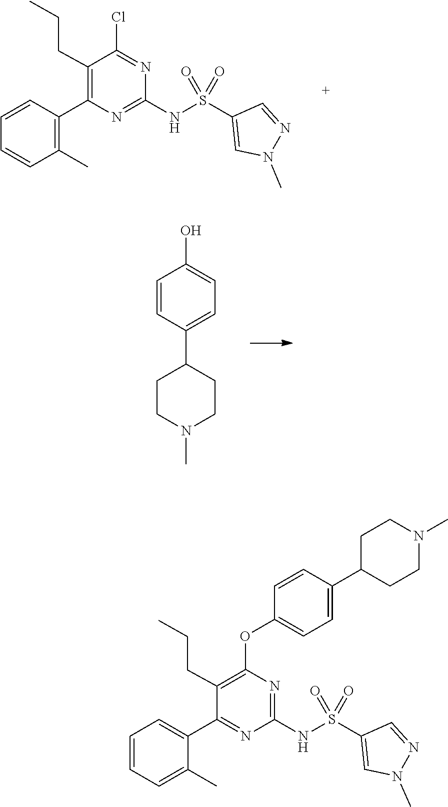 [SMILES: CCCc1c(Cl)nc(NS(=O)(=O)c2cnn(C)c2)nc1-c1ccccc1C ; CN1CCC(c2ccc(O)cc2)CC1 ; CCCc1c(Oc2ccc(C3CCN(C)CC3)cc2)nc(NS(=O)(=O)c2cnn(C)c2)nc1-c1ccccc1C]

A heterogeneous solution of N-[4-chloro-6-(o-tolyl)-5-propyl-pyrimidin-2-yl]-1 -methyl-pyrazole-4-sulfonamide (10.15 mg, 0.0250 mmol), 4-(1-methyl-4-piperidyl)phenol (approximately 9.563 mg, 0.05000 mmol), and potassium carbonate (approximately 10.37 mg, 0.07500 mmol) in NMP (50.00 μL) was heated in a sealed vial to 115° C. for 16 hours. The solution was acidified with acetic acid. The sample was purified by reverse phase HPLC (Waters Sunfire C₁₈ column (100×50 mm, 10 m particle size), gradient: 1-99% acetonitrile in water (5mM HCl) over 15.0 minutes) to afford 1-methyl-N-[4-[4-(1-methyl-4-piperidyl)phenoxy]-6-(o-tolyl)-5-propyl-pyrimidin-2-yl]pyrazole-4-sulfonamide (hydrochloride salt) as a white solid (9.3 mg, 66%). ESI-MS m/z calc. 560.25696, found 561.4 (M+1)⁺; Retention time: 1.47 minutes; LC method A.

### Example 447: Preparation of Compound 1484

### Step 1: N-[4-[2-chloro-3-(4-methylpiperazin-1-yl)phenoxy]-6-(o-tolyl)-5-propyl-pyrimidin-2-yl]-1-methyl-pyrazole-4-sulfonamide

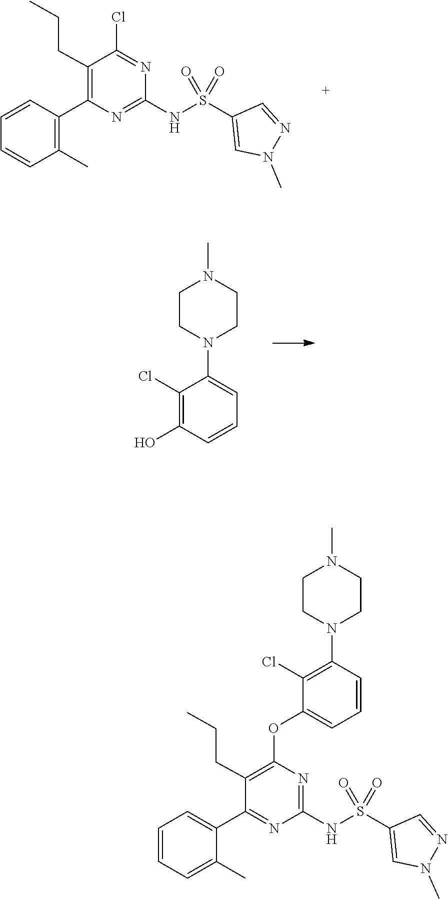 [SMILES: CCCc1c(Cl)nc(NS(=O)(=O)c2cnn(C)c2)nc1-c1ccccc1C ; CN1CCN(c2cccc(O)c2Cl)CC1 ; CCCc1c(Oc2cccc(N3CCN(C)CC3)c2Cl)nc(NS(=O)(=O)c2cnn(C)c2)nc1-c1ccccc1C]

The compound was prepared in a manner analogous to that described above using 2-chloro-3-(4-methylpiperazin-1-yl)phenol to give N-[4-[2-chloro-3-(4-methylpiperazin-1-yl)phenoxy]-6-(o-tolyl)-5-propyl-pyrimidin-2-yl]-1-methyl-pyrazole-4-sulfonamide (hydrochloride salt) as a white solid (6.3 mg). ESI-MS m/z calc. 595.21326, found 596.1 (M+1)⁺; Retention time: 1.37 minutes (LC method A).

### Example 449: Preparation of Compound 1485

### Step 1: 1-Methyl-N-[4-[3-(4-methylpiperazin-1-yl)phenoxy]-6-(o-tolyl)-5-propyl-pyrimidin-2-yl]pyrazole-4-sulfonamide

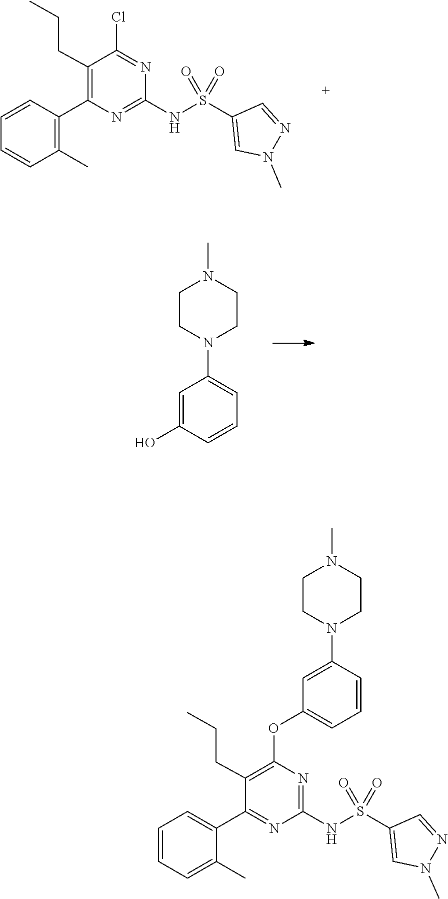 [SMILES: CCCc1c(Cl)nc(NS(=O)(=O)c2cnn(C)c2)nc1-c1ccccc1C ; CN1CCN(c2cccc(O)c2)CC1 ; CCCc1c(Oc2cccc(N3CCN(C)CC3)c2)nc(NS(=O)(=O)c2cnn(C)c2)nc1-c1ccccc1C]

The compound was prepared in a manner analogous to that described above using 3-(4-methylpiperazin-1-yl)phenol to give 1-methyl-N-[4-[3-(4-methylpiperazin-1-yl)phenoxy]-6-(o-tolyl)-5-propyl-pyrimidin-2-yl]pyrazole-4-sulfonamide (hydrochloride salt) as a white solid (7.2 mg). ESI-MS m/z calc. 561.2522, found 562.35 (M+1)⁺; Retention time: 1.45 minutes (LC method A).

### Example 450: Preparation of Compound 1486

### Step 1: 1-Methyl-N-[4-(o-tolyl)-6-phenoxy-5-propyl-pyrimidin-2-yl]pyrazole-4-sulfonamide

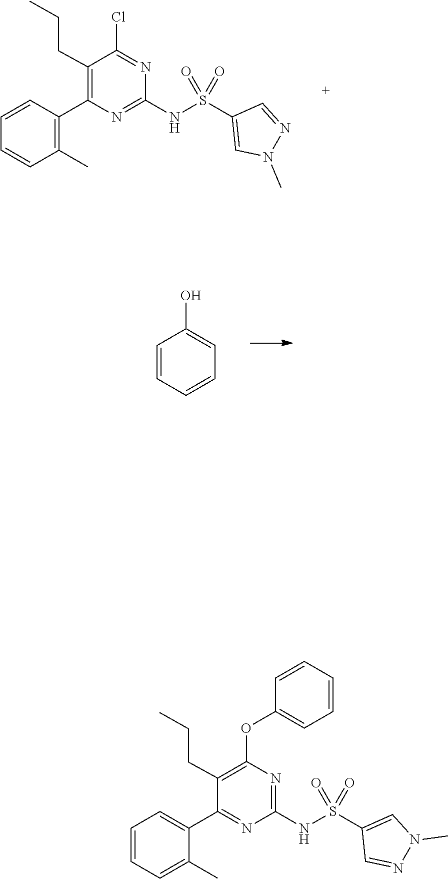 [SMILES: CCCc1c(Cl)nc(NS(=O)(=O)c2cnn(C)c2)nc1-c1ccccc1C ; Oc1ccccc1 ; CCCc1c(Oc2ccccc2)nc(NS(=O)(=O)c2cnn(C)c2)nc1-c1ccccc1C]

The compound was prepared in a manner analogous to that described above using commercially available phenol to give 1-methyl-N-[4-(o-tolyl)-6-phenoxy-5-propyl-pyrimidin-2-yl]pyrazole-4-sulfonamide (2.6 mg, 14%) as a white solid. ESI-MS m/z calc. 463.16782, found 464.1 (M+1)⁺; Retention time: 1.84 minutes; LC method A.

### Example 451: Preparation of Compound 1487

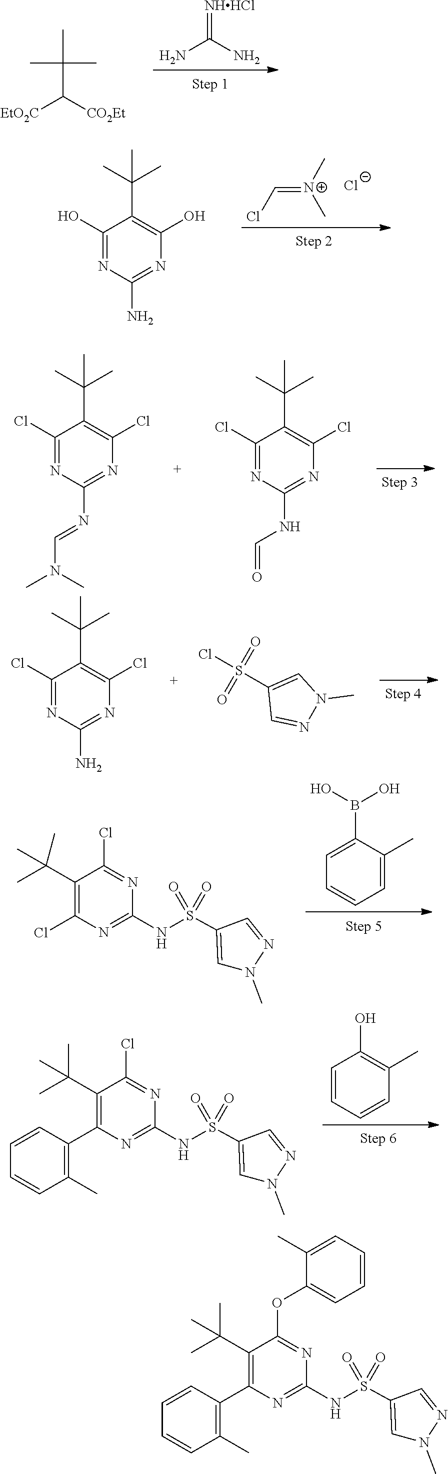

### Step 1: 2-Amino-5-tert-butyl-pyrimidine-4,6-diol

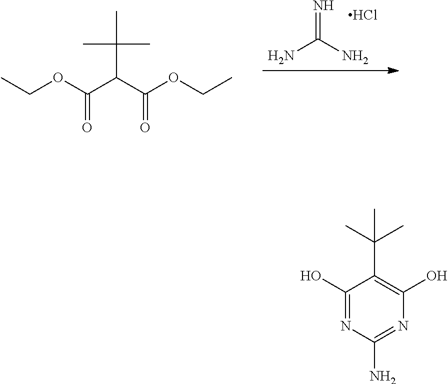

To a 2.0 L flask charged with ethanol (760 mL), solid chunks of sodium metal (17.7 g, 769.9 mmol) were added gradually and carefully and the mixture was stirred until all solids completely dissolved. Once cooled back to room temperature guanidine (hydrochloride salt) (28.3 g, 296.2 mmol) and diethyl 2-tert-butylpropanedioate (50 g, 231.2 mmol) were successively added and the reaction was heated at 80° C. for 17 hours. Once cooled to room temperature, the crude mixture was concentrated under reduced pressure to remove most of the ethanol. Water was added to complete dissolution (350 mL), the resulting solution was cooled in an ice bath and acidified to pH of 1-2 using concentrated HCl. The solids were filtered and washed with water (2×100 mL) then with acetone (2×100 mL) and dried under high vacuum to provide 2-amino-5-tert-butyl-pyrimidine-4,6-diol hydrate (43.4 g, 93%) as an off-white solid.

¹H NMR (300 MHz, DMSO-d₆) δ ppm 1.01 (s, 9H). ESI-MS m/z calc. 183.10078, found 184.2 (M+1)⁺; Retention time: 0.71 minutes; LC method C.

### Step 2: N′-(5-tert-Butyl-4,6-dichloro-pyrimidin-2-yl)-N,N-dimethyl-formamidine and N-(5-tert-butyl-4,6-dichloro-pyrimidin-2-yl)formamide

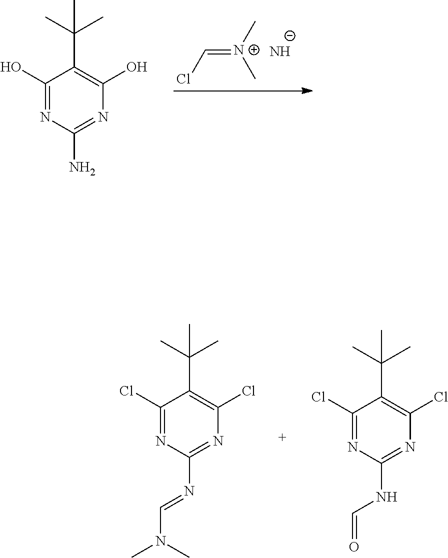

2-Amino-5-tert-butyl-pyrimidine-4,6-diol (2 g, 10.92 mmol) was dissolved in chloroform (40 mL) and (Chloromethylene)dimethyliminium chloride (11.3 g, 88.28 mmol) was added and the mixture was heated at reflux for 2 hours under nitrogen atmosphere then left to cool down and stirred overnight at room temperature. It was heated to reflux for another 4 hours. The reaction mixture was cooled to room temperature then partitioned between ice cold saturated NaHCO₃(50 mL) and dichloromethane (2×50 mL). The combined organics were dried over sodium sulfate then filtered and concentrated under reduced pressure. The resulting residue was purified on silica gel using 20 then 45% ethyl acetate in heptane to give mainly two products as white solids N′-(5-tert-butyl-4,6-dichloro-pyrimidin-2-yl)-N,N-dimethyl-formamidine (1.2 g, 40%) ESI-MS m/z calc. 275.178, found 275.1 (M+1)⁺; Retention time: 1.568 minutes (LC method C), ¹H NMR (300 MHz, CDCl₃) δ ppm 1.57-1.63 (s, 9H), 3.15 (d, J=2.1 Hz, 6H), 8.59 (s, 1H) and N-(5-tert-butyl-4,6-dichloro-pyrimidin-2-yl)formamide (600 mg, 22%), ESI-MS m/z calc. 248.109, found 248.1 (M+1)⁺; Retention time: 2.05 minutes (LC method C), ¹H NMR (300 MHz, CDCl₃) δ ppm 1.65 (s, 9H), 7.83 (d, J=8.8 Hz, 1H), 9.35 (d, J=10.6 Hz, 1H).

### Step 3: 5-tert-Butyl-4,6-dichloro-pyrimidin-2-amine

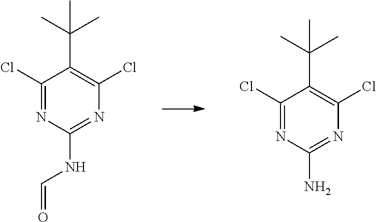

To N-(5-tert-butyl-4,6-dichloro-pyrimidin-2-yl)formamide (330 mg, 1.330 mmol) dissolved in isopropanol (7 mL) was added HCl (0.6 mL of 12 M, 7.200 mmol) and the mixture was stirred 30 minutes. at 50° C. The reaction mixture was concentrated under reduced pressure to provide the desired product 5-tert-butyl-4,6-dichloro-pyrimidin-2-amine (277 mg, 95%) as a white solid. ¹H NMR (300 MHz, CDCl₃) δ ppm 1.61 (s, 9H), 5.07 (br. s., 2H). ESI-MS m/z calc. 219.033, found 220.1 (M+1)⁺; Retention time: 2.048 minutes; LC method C.

### Step 4: N-(5-tert-butyl-4,6-dichloro-pyrimidin-2-yl)-1-methyl-pyrazole-4-sulfonamide

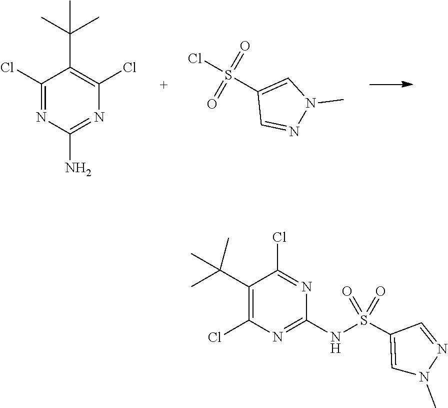

To a solution of 5-tert-butyl-4,6-dichloro-pyrimidin-2-amine (1.01 g, 4.589 mmol) in DMF (16 mL) at 0° C. was added sodium hydride (751 mg of 60% w/w, 18.78 mmol). 1-methylpyrazole-4-sulfonyl chloride (1.011 g, 5.598 mmol) was added and the reaction mixture was allowed to warm to room temperature and stir for 1 hour. The reaction mixture was cooled in an ice bath and acidified with a 1M HCl solution. The reaction was extracted with ethyl acetate (2×). The organic layer was washed with brine, dried over magnesium sulfate, filtered, and concentrated. The crude material was triturated with a 1:1 mixture of ether:hexanes and the resulting solid was collected and further dried to give N-(5-tert-butyl-4,6-dichloro-pyrimidin-2-yl)-1-methyl-pyrazole-4-sulfonamide (1.103 g, 66%). ESI-MS m/z calc. 363.03235, found 364.0 (M+1)⁺; Retention time: 0.62 minutes; LC method D.

### Step 5: N-[5-tert-butyl-4-chloro-6-(o-tolyl)pyrimidin-2-yl]-1-methyl-pyrazole-4-sulfonamide

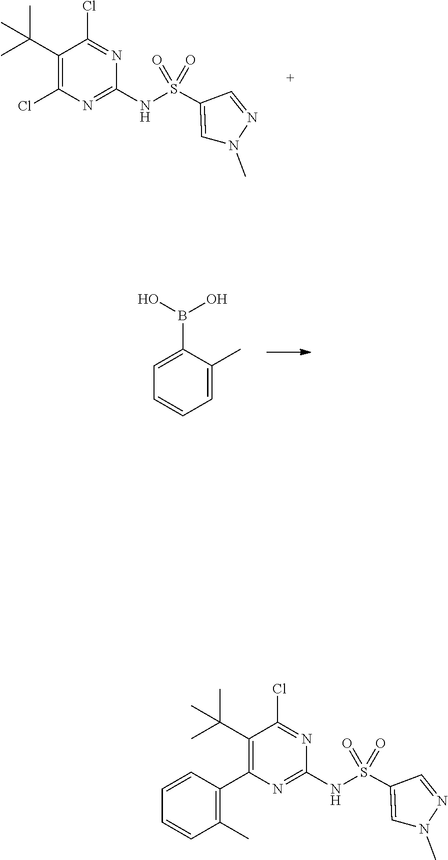

A heterogeneous mixture of N-(5-tert-butyl-4,6-dichloro-pyrimidin-2-yl)-1-methyl-pyrazole-4-sulfonamide (508 mg, 1.171 mmol), bis(triphenylphosphine)palladium(II) dichloride(82 mg, 0.1168 mmol), potassium carbonate (485 mg, 3.509 mmol), and o-tolylboronic acid (159 mg, 1.169 mmol) in dioxane (3.9 mL) and water (780 μL) was heated in a sealed vial to 85° C. for 16 hours. After cooling, the dioxane layer was removed under a steady stream of air. Acetic acid (1.3 mL, 22.86 mmol) and water were added. The solution was stirred until the base was neutralized and then ethyl acetate was added. The organic layer was removed, and the aqueous layer was further extracted once with ethyl acetate. The combined organic extracts were concentrated in vacuo. The crude residue was separated by flash column chromatography on silica gel (gradient: 10 to 50% ethyl acetate in hexanes) to afford N-[5-tert-butyl-4-chloro-6-(o-tolyl)pyrimidin-2-yl]-1-methyl-pyrazole-4-sulfonamide (350 mg, 71%) as a white solid. ESI-MS m/z calc. 419.1183, found 420.1 (M+1)⁺; Retention time: 0.71 minutes; LC method D.

### Step 6: N-[5-tert-butyl-4-(2-methylphenoxy)-6-(o-tolyl)pyrimidin-2-yl]-1-methyl-pyrazole-4-sulfonamide

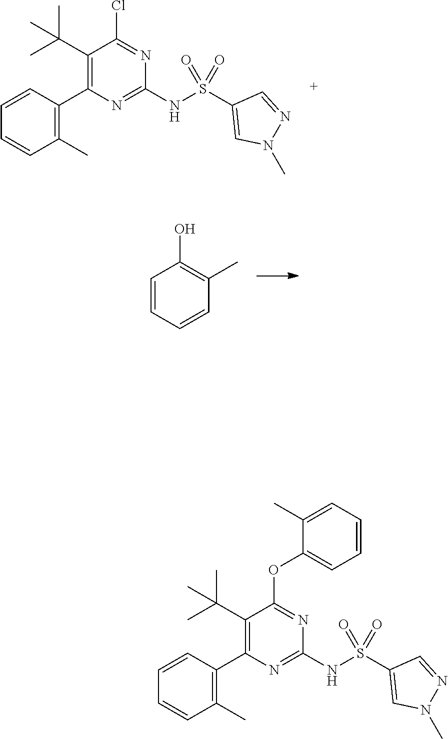

A heterogeneous solution consisting of o-cresol (12.98 mg, 23.47 μL, 0.1200 mmol), N-[5-tert-butyl-4-chloro-6-(o-tolyl)pyrimidin-2-yl]-1-methyl-pyrazole-4-sulfonamide (16.80 mg, 0.0400 mmol), and potassium carbonate (approximately 7.369 mg, 0.05332 mmol) in NMP was heated in a sealed vial to 110° C. for 16 hours. The solution was acidified using acetic acid (approximately 24.02 mg, 22.75 μL, 0.4000 mmol), diluted with DMSO (0.5 mL), and filtered using a 0.2 μM PTFE syringe filter. The crude solution was separated by preparative HPLC (gradient: 10 to 99% acetontirle in water with 0.1% hydrochloric acid) which furnished N-[5-tert-butyl-4-(2-methylphenoxy)-6-(o-tolyl)pyrimidin-2-yl]-1-methyl-pyrazole-4-sulfonamide (8.6 mg, 44%) as a white solid. ESI-MS m/z calc. 491.1991, found 492.1 (M+1)⁺; Retention time: 1.96 minutes; LC method A.

### Example 452: Preparation of Compound 1488

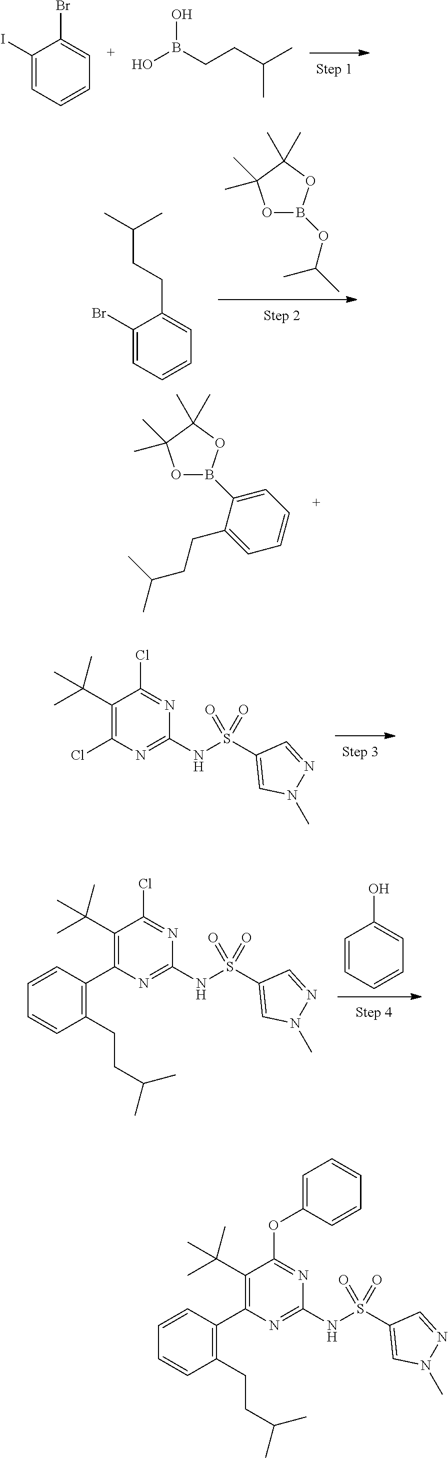

### Step 1: 1-Bromo-2-isopentyl-benzene

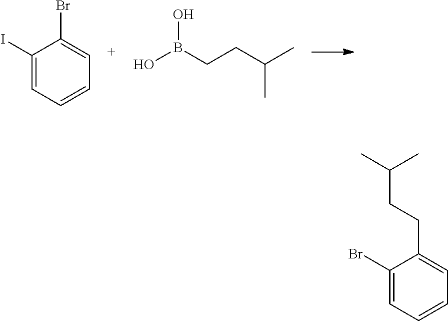

A suspension of 1-bromo-2-iodo-benzene (5.5075 g, 2.5 mL, 19.468 mmol), 3-methylbutylboronic acid (3.4 g, 29.319 mmol) and potassium phosphate (8.3 g, 39.102 mmol) in dry dioxane (75 mL) was degassed for 10 minutes by bubbling nitrogen. Pd(dppf)C₁₂ (1.6 g, 1.9593 mmol) was added. The reaction was sparged for another 5 minutes and stirred at reflux overnight. The reaction was cooled to room temperature and 3-methylbutylboronic acid (565 mg, 4.8721 mmol) and Pd(dppf)C₁₂ (318 mg, 0.3894 mmol) were added. The reaction was sparged for 10 minutes and stirred at reflux overnight. The reaction was cooled down to room temperature and partitioned between water (75 mL) and EtOAc (100 mL). The mixture was filtered, and the aqueous layer was extracted with EtOAc (2×50 mL). The combined organic layers were washed with brine (2×50 mL), dried over anhydrous sodium sulfate, filtered and concentrated under reduced pressure. The product was purified by a silica plug eluted with heptane to yield 1-bromo-2-isopentyl-benzene (3.28 g, 74%) as a colorless oil. ¹H NMR (300 MHz, CDCl₃) δ ppm 0.97 (d, J=6.5 Hz, 6H), 1.41-1.53 (m, 2H), 1.63 (dq, J=13.2, 6.6 Hz, 1H), 2.67-2.76 (m, 2H), 6.97-7.08 (m, 1H), 7.14-7.24 (m, 2H), 7.46-7.56 (m, 1H). ESI-MS m/z calc. 226.0357, not ionized, Retention time: 2.57 minutes; LC method C.

### Step 2: 2-(2-Isopentylphenyl)-4,4,5,5-tetramethyl-1,3,2-dioxaborolane

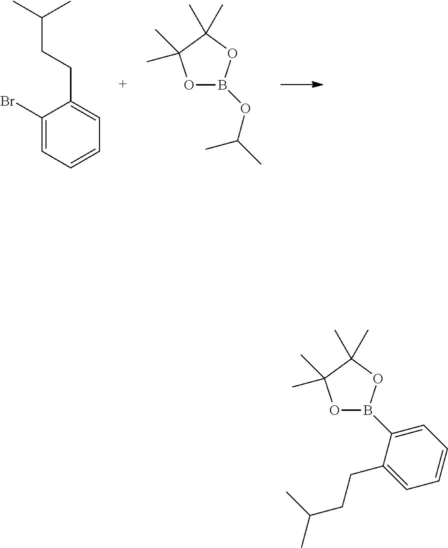 [SMILES: CC(C)CCc1ccccc1Br ; CC(C)OB1OC(C)(C)C(C)(C)O1 ; CC(C)CCc1ccccc1B1OC(C)(C)C(C)(C)O1]

n-Butyllithium (4.2 mL of 2.5 M in hexanes, 10.500 mmol) was slowly added to a solution of 1-bromo-2-isopentyl-benzene (1.95 g, 8.5850 mmol) in tetrahydrofuran (40 mL) at −78° C. After stirring for 30 minutes at the same temperature, 2-isopropoxy-4,4,5,5-tetramethyl-1,3,2-dioxaborolane (1.9110 g, 2.10 mL, 10.271 mmol) was added and the reaction mixture was stirred at −78° C. for 2.5 hours. The reaction was quenched with water and diluted using ethyl acetate (100 mL). Layers were separated, the organic phase was washed with brine (50 mL), dried over sodium sulfate and concentrated under reduced pressure. The residue was purified by silica gel chromatography using 0% to 10% of EtOAc in heptanes to afford 2-(2-isopentylphenyl)-4,4,5,5-tetramethyl-1,3,2-dioxaborolane (1.41 g, 60%) as clear oil. ¹H NMR (300 MHz, CDCl₃) δ ppm 0.90-0.97 (m, 6H), 1.32-1.36 (m, 12H), 1.38-1.50 (m, 2H), 1.57-1.70 (m, 1H), 2.81-2.91 (m, 2H), 7.09-7.21 (m, 2H), 7.28-7.39 (m, 1H), 7.72-7.80 (m, 1H). ESI-MS m/z calc. 274.21042, not ionized, Retention time: 2.7 minutes; LC method C.

### Step 3: N-[5-tert-butyl-4-chloro-6-(2-isopentylphenyl)pyrimidin-2-yl]-1-methyl-pyrazole-4-sulfonamide

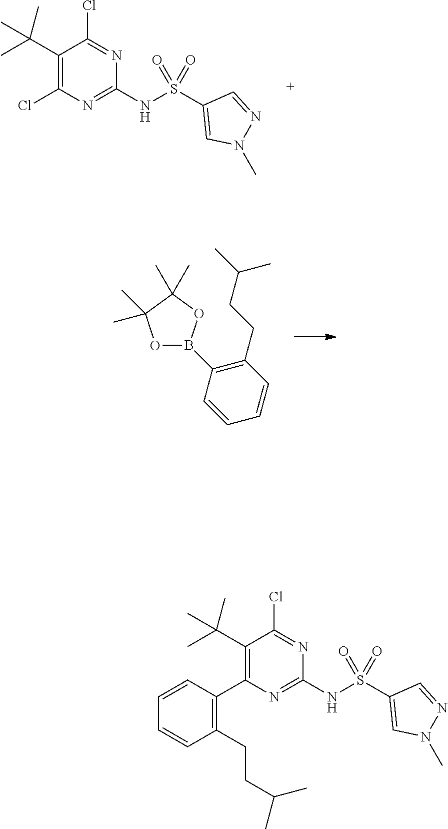 [SMILES: Cn1cc(S(=O)(=O)Nc2nc(Cl)c(C(C)(C)C)c(Cl)n2)cn1 ; CC(C)CCc1ccccc1B1OC(C)(C)C(C)(C)O1 ; CC(C)CCc1ccccc1-c1nc(NS(=O)(=O)c2cnn(C)c2)nc(Cl)c1C(C)(C)C]

A dioxane (0.8 mL) mixture of 2-(2-isopentylphenyl)-4,4,5,5-tetramethyl-1,3,2-dioxaborolane (91.3 mg, 0.3330 mmol), N-(5-tert-butyl-4,6-dichloro-pyrimidin-2-yl)-1-methyl-pyrazole-4-sulfonamide (157.6 mg, 0.4327 mmol), K₂CO₃ (500 μL of 2 M, 1.000 mmol), and Pd(PPh₃)₄ (77.9 mg, 0.06741 mmol) was microwaved at 120° C. for 30 minutes. The solution was filtered and the filtrate diluted with 0.8 mL MeOH, and purified by reverse phase chromatography using a 15 minute gradient of 1% MeCN in water to 99% MeCN (HCl modifier) to afford N-[5-tert-butyl-4-chloro-6-(2-isopentylphenyl)pyrimidin-2-yl]-1-methyl-pyrazole-4-sulfonamide (30.2 mg, 19%). ESI-MS m/z calc. 475.18088, found 476.3 (M+1)⁺; Retention time: 2.26 minutes; LC method A.

### Step 4: N-[5-tert-butyl-4-(2-isopentylphenyl)-6-phenoxy-pyrimidin-2-yl]-1-methyl-pyrazole-4-sulfonamide

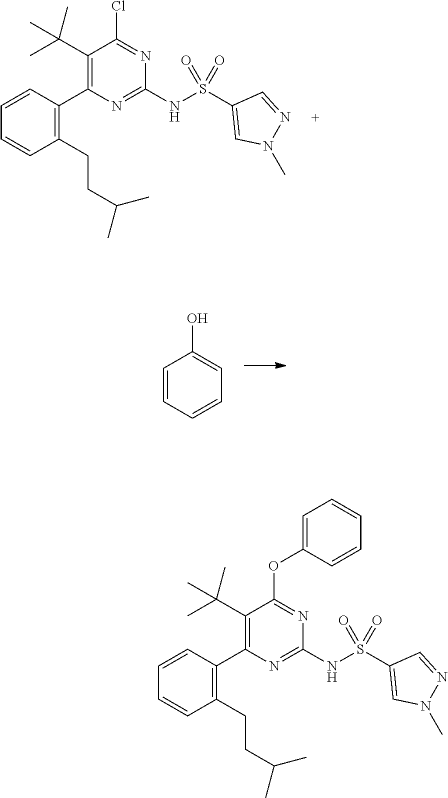 [SMILES: CC(C)CCc1ccccc1-c1nc(NS(=O)(=O)c2cnn(C)c2)nc(Cl)c1C(C)(C)C ; Oc1ccccc1 ; CC(C)CCc1ccccc1-c1nc(NS(=O)(=O)c2cnn(C)c2)nc(Oc2ccccc2)c1C(C)(C)C]

A NMP (0.5 mL) mixture of N-[5-tert-butyl-4-chloro-6-(2-isopentylphenyl)pyrimidin-2-yl]-1-methyl-pyrazole-4-sulfonamide (6.5 mg, 0.01365 mmol), phenol (6.1 mg, 0.06482 mmol), and Cs₂CO₃ (31.2 mg, 0.09576 mmol) was heated at 130° C. for 16 hours. The solution was filtered and the filtrate was diluted with 0.8 mL MeOH, and purified by reverse phase chromatography using a 15 minute gradient of 1% MeCN in water to 99% MeCN (HCl modifier) to give N-[5-tert-butyl-4-(2-isopentylphenyl)-6-phenoxy-pyrimidin-2-yl]-1-methyl-pyrazole-4-sulfonamide (2.4 mg, 33%). ESI-MS m/z calc. 533.24603, found 534.4 (M+1)⁺; Retention time: 2.32 minutes; LC method A.

### Example 453: Preparation of 1489

### Step 1: N-[5-tert-butyl-4-(2-isopentylphenyl)-6-(2-isopropoxyphenoxy)pyrimidin-2-yl]-1-methyl-pyrazole-4-sulfonamide

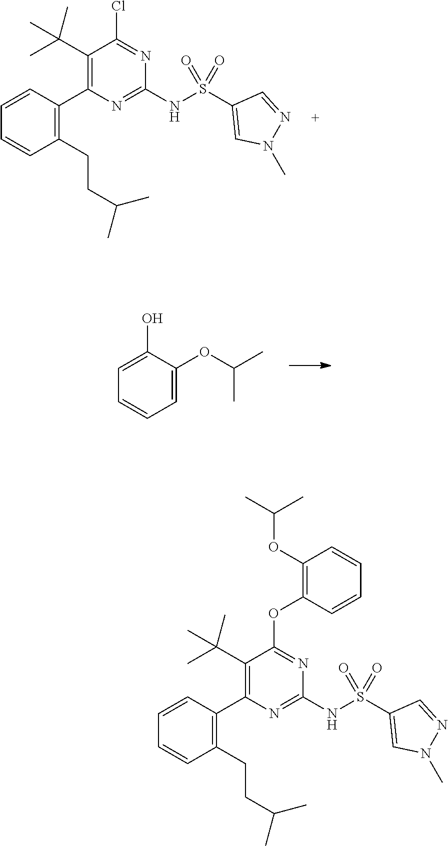 [SMILES: CC(C)CCc1ccccc1-c1nc(NS(=O)(=O)c2cnn(C)c2)nc(Cl)c1C(C)(C)C ; CC(C)Oc1ccccc1O ; CC(C)CCc1ccccc1-c1nc(NS(=O)(=O)c2cnn(C)c2)nc(Oc2ccccc2OC(C)C)c1C(C)(C)C]

A NMP (0.5 mL) mixture of 2-isopropoxyphenol (8.2 mg, 0.05388 mmol), N-[5-tert-butyl-4-chloro-6-(2-isopentylphenyl)pyrimidin-2-yl]-1-methyl-pyrazole-4-sulfonamide (7.6 mg, 0.01597 mmol), and Cs₂CO₃ (21.7 mg, 0.06660 mmol) was stirred at 140° C. for 16 hours and then cooled to room temperature. The solution was filtered and the filtrate was diluted in 0.8 mL of MeOH, and purified by reverse phase chromatography using a 15 min gradient of 1% MeCN in water to 99% MeCN (HCl modifier) to give N-[5-tert-butyl-4-(2-isopentylphenyl)-6-(2-isopropoxyphenoxy)pyrimidin-2-yl]-1-methyl-pyrazole-4-sulfonamide (2.4 mg, 25%). ESI-MS m/z calc. 591.2879, found 592.2 (M+1)⁺; Retention time: 2.49 minutes; LC method A.

### Example 454: Preparation of Compound 1490

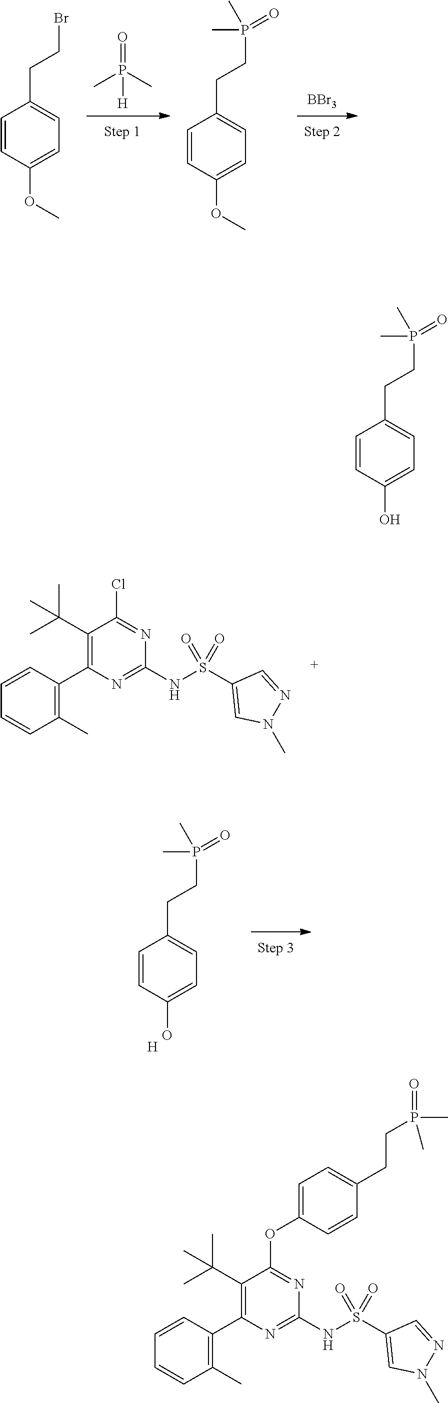

### Step 1: 1-(2-Dimethylphosphorylethyl)₄-methoxy-benzene

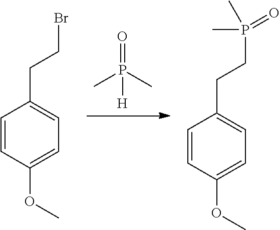

To a solution of methylphosphonoylmethane (145 mg, 1.858 mmol) in THE (3.7 mL) was added sodium hydride (112 mg of 60% w/w, 2.800 mmol). The reaction was allowed to stir for 30 min before adding 1-(2-bromoethyl)-4-methoxy-benzene (400.0 mg, 1.860 mmol) in one portion. The reaction was stirred for 16 hours and was acidified with acetic acid (650 μL, 11.43 mmol) and concentrated to a 20% volume of solvent under a steady stream of air. The crude solution was diluted with DMSO (0.5 mL) and the sample was purified by reverse phase HPLC (Phenomenex Luna C₁₈ column (75×30 mm, 5 m particle size), gradient: 1-99% acetonitrile in water (5 mM HCl) over 15.0 minutes) to afford 1-(2-dimethylphosphorylethyl)-4-methoxy-benzene (36 mg, 9%) as a white solid. ESI-MS m/z calc. 212.09662, found 213.07 (M+1)⁺; Retention time: 0.34 minutes; LC method D.

### Step 2: 4-(2-Dimethylphosphorylethyl)phenol

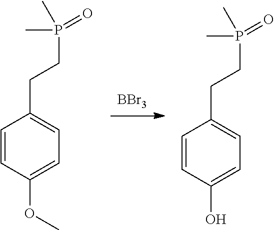

To a solution of 1-(2-dimethylphosphorylethyl)-4-methoxy-benzene (36 mg, 0.1696 mmol) in DCM (450 μL) at 0° C. was slowly added tribromoborane (340 μL of 1 M in DCM, 0.3400 mmol). The reaction mixture was allowed to stir for 16 hours. The reaction was cooled to 0° C. and quenched with water. Dichloromethane was removed under a slow stream of air blown over the surface. The resulting solution was diluted with DMSO. The sample was purified by reverse phase HPLC (Phenomenex Luna C₁₈ column (75×30 mm, 5 m particle size), gradient: 1-99% acetonitrile in water (5 mM HCl) over 15.0 minutes) to afford 4-(2-dimethylphosphorylethyl)phenol (26 mg, 77%) as a white solid. ESI-MS m/z calc. 198.08096, found 199.06 (M+1)⁺; Retention time: 0.25 minutes; LC method D.

### Step 3: N-[5-tert-butyl-4-[4-(2-dimethylphosphorylethyl)phenoxy]-6-(o-tolyl)pyrimidin-2-yl]-1-methyl-pyrazole-4-sulfonamide

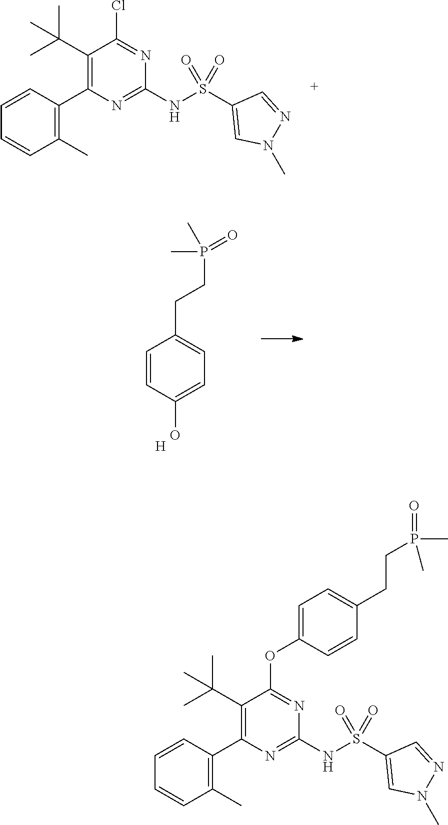

A heterogeneous solution of 4-(2-dimethylphosphorylethyl)phenol (13.0 mg, 0.06559 mmol), N-[5-tert-butyl-4-chloro-6-(o-tolyl)pyrimidin-2-yl]-1-methyl-pyrazole-4-sulfonamide (23 mg, 0.05477 mmol), and potassium carbonate (22.7 mg, 0.1642 mmol) in NMP (110 μL) was heated in a sealed vial at 150° C. for 5 hours. The reaction was cooled, acidified with acetic acid (32.9 mg, 0.5479 mmol) and diluted with DMSO (0.5 mL). The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[5-tert-butyl-4-[4-(2-dimethylphosphorylethyl)phenoxy]-6-(o-tolyl)pyrimidin-2-yl]-1-methyl-pyrazole-4-sulfonamide (3.0 mg, 9%) as a white solid. ESI-MS m/z calc. 581.22253, found 582.3 (M+1)⁺; Retention time: 1.58 minutes; LC method A.

### Example 455: Preparation of Compound 1491

### Step 1: 3-(1-Methylpyridin-1-ium-4-yl)phenol iodide

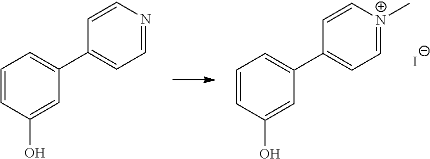

A solution of 3-pyridin-4-yl-phenol (9.27 g, 54.1 mmol) and methyl iodide (13 mL, 216.6 mmol) in acetone (550 mL) was heated at 65° C. for overnight. The reaction mixture was concentrated in vacuum to half volume and the solid was filtered-off, rinsed with acetone to give 4-(3-hydroxy-phenyl)-1-methyl-pyridinium iodide (16.02 g, 95%) as a yellow solid. ESI-MS m z calc. 312.99637, found 185.7 (M+1)⁺; Retention time: 1.29 minutes.

### Step 2: 3-(1-Methyl-4-piperidyl)phenol

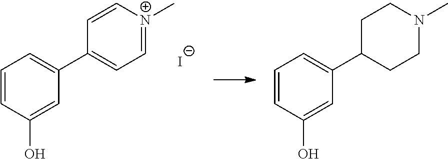

Approximately 5 g of Raney Nickel was added to 500 mL of methanol, followed by the addition of 4-(3-hydroxy-phenyl)-1-methyl-pyridinium iodide (16.02 g, 51.2 mmol). The reaction was hydrogenated on a Parr shaker under 60 PSI of hydrogen for 48 hours. Additional Raney Nickel (5 g ×2) was added after 16 and 32 hours shaking. The reaction mixture was filtered over Celite, rinsed with methanol and concentrated under vacuum. Water (50 mL), a saturated aqueous sodium bicarbonate solution (100 mL) and brine (10 mL) were added to the crude and it was extracted with a mixture of 4:1 dichloromethane: iso-propanol (150 mL×6). The combined organic layer was dried over anhydrous magnesium sulfate and purified by silica gel chromatography using 0-20% dichloromethane-methanol (containing 1% triethylamine). Fractions were concentrated in vacuo and a 1 M aqueous sodium hydroxide solution was added. The aqueous layer was extracted with ethyl acetate (100 mL×5), acidified with 2 M aqueous hydrogen chloride solution to pH 5 and then neutralized with solid sodium bicarbonate. The aqueous layer was extracted with a mixture 4:1 dichloromethane: iso-propanol (100 mL×10) to afford 3-(1-methyl-piperidin-4-yl)-phenol (5.10 g, 52%) as a white solid. ¹H NMR (250 MHz, ACN-d₃) δ ppm 1.30-1.41 (m, 3H) 1.49-1.55 (m, 2H) 1.68-1.81 (m, 1H) 1.89 (s, 3H) 1.97 -2.13 (m, 1H) 2.50-2.68 (m, 2H) 6.17-6.36 (m, 3H) 6.70 (t, J=7.86 Hz, 1H). ESI-MS m z calc. 191.13101, found 192.0 (M+1)⁺; Retention time: 1.27 minutes.

### Step 3: N-[5-tert-butyl-4-[3-(1-methyl-4-piperidyl)phenoxy]-6-(o-tolyl)pyrimidin-2-yl]-1-methyl-pyrazole-4-sulfonamide

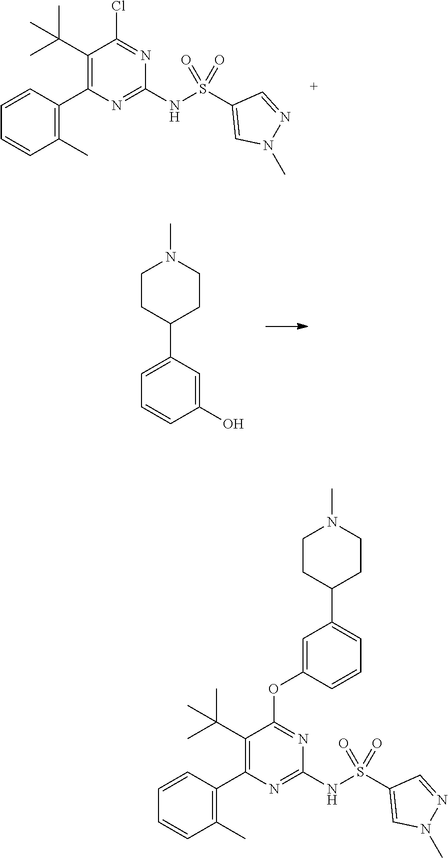 [SMILES: Cc1ccccc1-c1nc(NS(=O)(=O)c2cnn(C)c2)nc(Cl)c1C(C)(C)C ; CN1CCC(c2cccc(O)c2)CC1 ; Cc1ccccc1-c1nc(NS(=O)(=O)c2cnn(C)c2)nc(Oc2cccc(C3CCN(C)CC3)c2)c1C(C)(C)C]

A NMP (1 mL) mixture of N-[5-tert-butyl-4-chloro-6-(o-tolyl)pyrimidin-2-yl]-1 -methyl-pyrazole-4-sulfonamide (0.0501 g, 0.1193 mmol), 3-(1-methyl-4-piperidyl)phenol (67.5 mg, 0.3529 mmol), and cesium carbonate (170.3 mg, 0.5227 mmol) was stirred at 140° C. for 18 hours and then cooled to room temperature. The solution was filtered and the filtrate dissolved in 0.8 mL MeOH, and purified by reverse phase chromatography using a 15 minute gradient of 1% MeCN in water to 99% MeCN (HCl modifier) to give N-[5-tert-butyl-4-[3-(1-methyl-4-piperidyl)phenoxy]-6-(o-tolyl)pyrimidin-2-yl]-1-methyl-pyrazole-4-sulfonamide (hydrochloride salt) (4.7 mg, 6%). ESI-MS m/z calc. 574.2726, found 575.37 (M+1)⁺; Retention time: 1.4 minutes; LC method A.

### Example 456: Preparation of Compound 1492

### Step 1: 3-(4-Methylpiperazin-1-yl)phenol

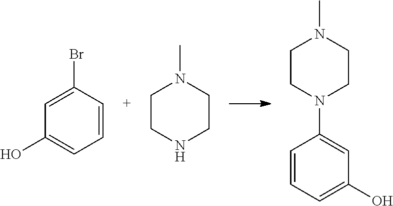

In a glass vial, 3-bromophenol (51.9 mg, 0.300 mmol), [2-(2-aminoethyl)phenyl]-chloro-palladium;ditert-butyl-[2-(2,4,6-triisopropylphenyl)phenyl]phosphane (XPhos Pd G1)(26.5 mg, 0.0386 mmol), dioxane (800 μL), 1-methylpiperazine (100 μL), and sodium tert-butoxide (60.4 mg, 0.628 mmol) were combined and the mixture was sparged with nitrogen for 5 minutes. The reaction was stirred at 35° C. for 30 minutes. The solution was filtered and the resulting residue was dissolved in 1.2 mL of DMSO/MeOH (1:1) and purified by reverse phase chromatography using a 15 min gradient of 1% MeCN in water to 99% MeCN (HCl modifier) to give 3-(4-methylpiperazin-1-yl)phenol (hydrochloride salt) (66.3 mg, 97%). ESI-MS m/z calc. 192.12627, found 193.29 (M+1)⁺; Retention time: 0.63 minutes; LC method A.

### Step 2: N-[5-tert-butyl-4-[3-(4-methylpiperazin-1-yl)phenoxy]-6-(o-tolyl)pyrimidin-2-yl]-1-methyl-pyrazole-4-sulfonamide

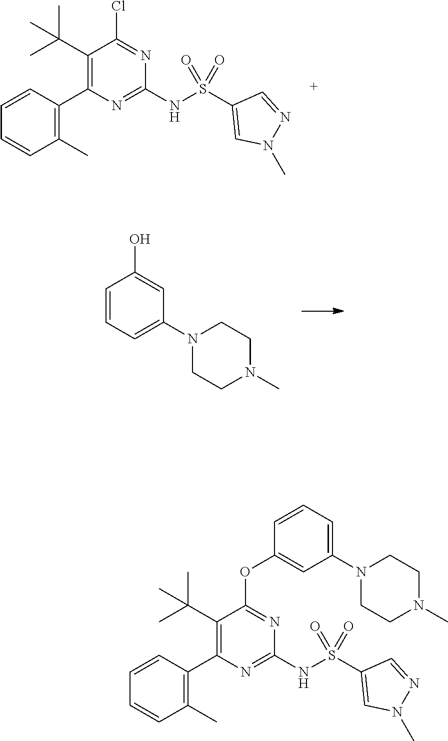 [SMILES: Cc1ccccc1-c1nc(NS(=O)(=O)c2cnn(C)c2)nc(Cl)c1C(C)(C)C ; CN1CCN(c2cccc(O)c2)CC1 ; Cc1ccccc1-c1nc(NS(=O)(=O)c2cnn(C)c2)nc(Oc2cccc(N3CCN(C)CC3)c2)c1C(C)(C)C]

A mixture of N-[5-tert-butyl-4-chloro-6-(o-tolyl)pyrimidin-2-yl]-1-methyl-pyrazole-4-sulfonamide (20.2 mg, 0.04810 mmol), 3-(4-methylpiperazin-1-yl)phenol (29.7 mg, 0.1545 mmol), and Cs₂CO₃ (63.6 mg, 0.1952 mmol) in NMP (0.5 mL) was stirred at 140° C. for 6 hours and then cooled to room temperature. The solution was filtered and the filtrate dissolved in 0.8 mL MeOH, and purified by reverse phase chromatography using a 15 minute gradient of 20% MeCN in water to 80% MeCN (HCl modifier) to give N-[5-tert-butyl-4-[3-(4-methylpiperazin-1-yl)phenoxy]-6-(o-tolyl)pyrimidin-2-yl]-1-methyl-pyrazole-4-sulfonamide (hydrochloride salt) (10.2 mg, 35%). ESI-MS m/z calc. 575.2679, found 576.36 (M+1)⁺; Retention time: 1.41 minutes; LC method A.

### Example 457: Preparation of Compound 1493

### Step 1: 2-Chloro-3-(4-methylpiperazin-1-yl)phenol

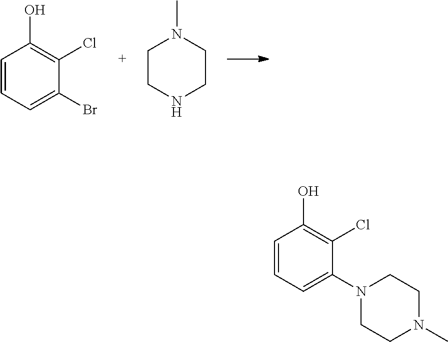

A heterogeneous mixture of 3-bromo-2-chloro-phenol (4.20 g, 20.25 mmol), 1-methylpiperazine (21.5 g, 214.7 mmol), chloro(2-di-t-butylphosphino-2′, 4′, 6′-tri-i-propyl-1,1′-biphenyl)[2-(2-aminoethyl)phenyl] palladium(II) [t-BuXPhos Palladacycle Gen. 1] (2.1 g, 3.058 mmol), and potassium tert-butoxide (4.8 g, 42.78 mmol) in dioxane (120 mL) was sonicated for 15 min and heated in a sealed vessel at 50° C. for 16 hours. The reaction mixture was acidified using acetic acid (3.4 mL, 59.79 mmol) then partitioned between DCM (100 mL) and water (100 mL). The organic layer was separated, and the aqueous layer was further extracted with DCM (4×). The combined organics were washed once with brine, dried using magnesium sulfate, filtered, and concentrated in vacuo. The crude residue was purified by flash column chromatography on silica gel (gradient: 1 to 10% methanol in dichloromethane) to give 2-chloro-3-(4-methylpiperazin-1-yl)phenol (3.86 g, 84%) as a yellow solid. ESI-MS m/z calc. 226.0873, found 227.12 (M+1)⁺; Retention time: 0.24 minutes; LC method D.

### Step 2: N-[5-tert-butyl-4-[2-chloro-3-(4-methylpiperazin-1-yl)phenoxy]-6-(o-tolyl)pyrimidin-2-yl]-1-methyl-pyrazole-4-sulfonamide

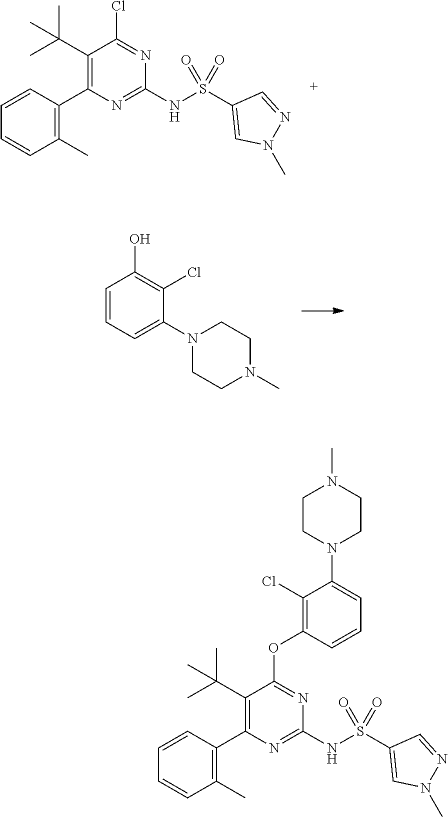 [SMILES: Cc1ccccc1-c1nc(NS(=O)(=O)c2cnn(C)c2)nc(Cl)c1C(C)(C)C ; CN1CCN(c2cccc(O)c2Cl)CC1 ; Cc1ccccc1-c1nc(NS(=O)(=O)c2cnn(C)c2)nc(Oc2cccc(N3CCN(C)CC3)c2Cl)c1C(C)(C)C]

A) mixture of N-[5-tert-butyl-4-chloro-6-(o-tolyl)pyrimidin-2-yl]-1-methyl-pyrazole-4-sulfonamide (20.2 mg, 0.04810 mmol), 2-chloro-3-(4-methylpiperazin-1-yl)phenol (33.1 mg, 0.1460 mmol), Cs₂CO₃ (63.6 mg, 0.1952 mmol) in NMP (0.5 mL was stirred at 140° C. for 6 hours and then cooled to room temperature. The solution was filtered and the filtrate dissolved in 0.8 mL MeOH, and purified by reverse phase chromatography using a 15 minute gradient of 20% MeCN in water to 80% MeCN (HCl modifier) to give N-[5-tert-butyl-4-[2-chloro-3-(4-methylpiperazin-1-yl)phenoxy]-6-(o-tolyl)pyrimidin-2-yl]-1-methyl-pyrazole-4-sulfonamide (hydrochloride salt) (6.2 mg, 20%). ESI-MS m/z calc. 609.2289, found 610.36 (M+1)⁺; Retention time: 1.46 minutes; LC method A.

### Example 458: Preparation of Compound 1494

### Step 1: N-[5-tert-butyl-4-(2-isopropylphenoxy)-6-(o-tolyl)pyrimidin-2-yl]-1-methyl-pyrazole-4-sulfonamide

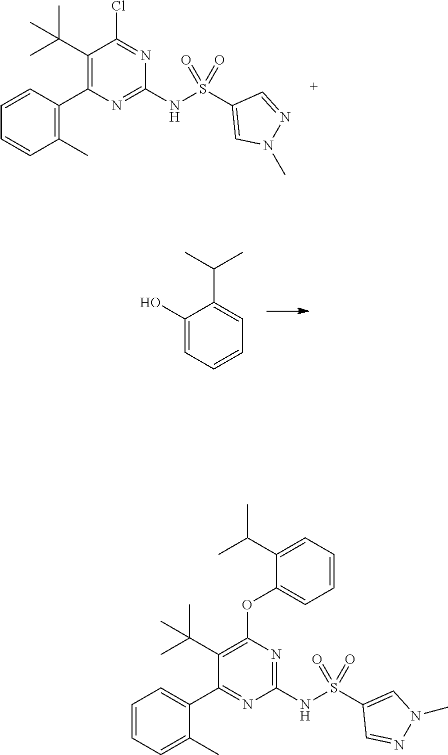

The compound was prepared in a manner analogous to that described above using commercially available 2-isopropylphenol to give N-[5-tert-butyl-4-(2-isopropylphenoxy)-6-(o-tolyl)pyrimidin-2-yl]-1-methyl-pyrazole-4-sulfonamide (6.6 mg, 32%) as a white solid. ESI-MS m/z calc. 519.2304, found 520.2 (M+1)⁺; Retention time: 2.15 minutes; LC method A.

### Example 459: Preparation of Compound 1495

### Step 1: N-[5-tert-butyl-4-(2-chlorophenoxy)-6-(o-tolyl)pyrimidin-2-yl]-1-methyl-pyrazole-4-sulfonamide

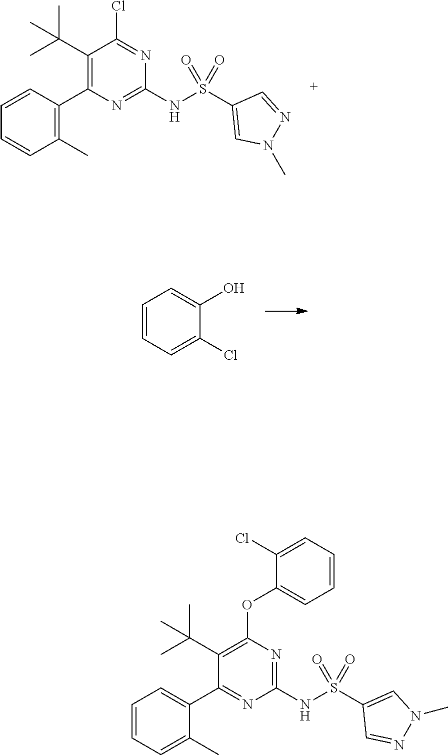 [SMILES: Cc1ccccc1-c1nc(NS(=O)(=O)c2cnn(C)c2)nc(Cl)c1C(C)(C)C ; Oc1ccccc1Cl ; Cc1ccccc1-c1nc(NS(=O)(=O)c2cnn(C)c2)nc(Oc2ccccc2Cl)c1C(C)(C)C]

The compound was prepared in a manner analogous to that described above using commercially available 2-chlorophenol to give N-[5-tert-butyl-4-(2-chlorophenoxy)-6-(o-tolyl)pyrimidin-2-yl]-1-methyl-pyrazole-4-sulfonamide (10.2 mg, 47%) as a white solid. ESI-MS m/z calc. 511.1445, found 512.1 (M+1)⁺; Retention time: 1.98 minutes; LC method A.

### Example 460: Preparation of Compound 1496

### Step 1: N-[5-tert-butyl-4-[4-(1-methyl-4-piperidyl)phenoxy]-6-(o-tolyl)pyrimidin-2-yl]-1-methyl-pyrazole-4-sulfonamide

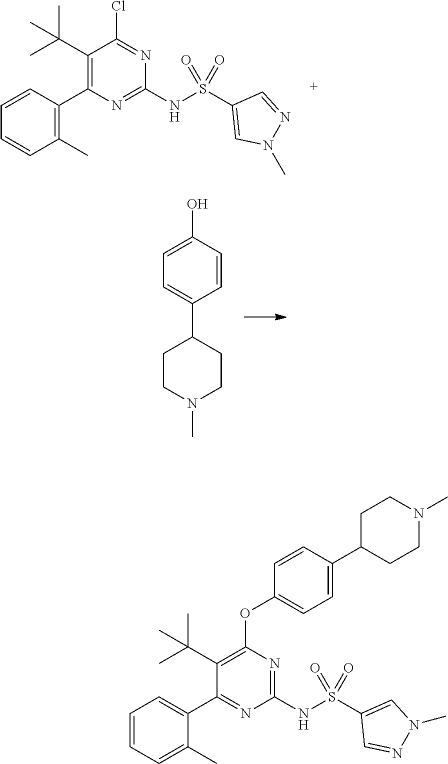 [SMILES: Cc1ccccc1-c1nc(NS(=O)(=O)c2cnn(C)c2)nc(Cl)c1C(C)(C)C ; CN1CCC(c2ccc(O)cc2)CC1 ; Cc1ccccc1-c1nc(NS(=O)(=O)c2cnn(C)c2)nc(Oc2ccc(C3CCN(C)CC3)cc2)c1C(C)(C)C]

The compound was prepared in a manner analogous to that described above using 4-(1-methyl-4-piperidyl)phenol to give N-[5-tert-butyl-4-[4-(1-methyl-4-piperidyl)phenoxy]-6-(o-tolyl)pyrimidin-2-yl]-1-methyl-pyrazole-4-sulfonamide (hydrochloride salt) (9.3 mg, 40%) as a white solid. ESI-MS m/z calc. 574.2726, found 575.2 (M+1)⁺; Retention time: 1.38 minutes; LC method A.

### Example 461: Preparation of Compound 1497

### Step 1: N-[5-tert-butyl-4-[4-(4-methylpiperazin-1-yl)phenoxy]-6-(o-tolyl)pyrimidin-2-yl]-1-methyl-pyrazole-4-sulfonamide

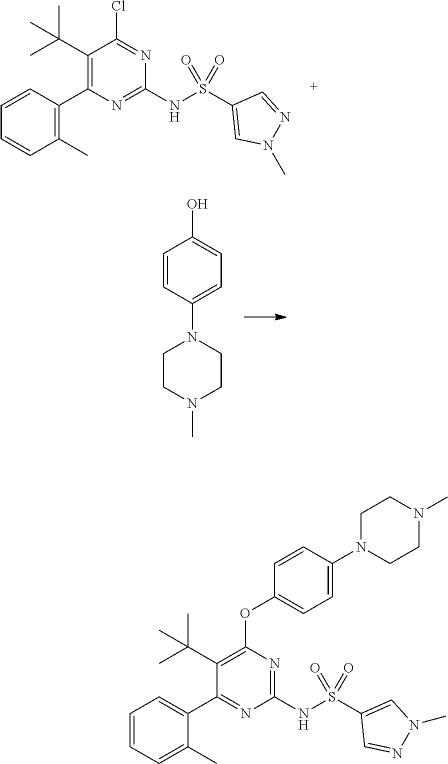 [SMILES: Cc1ccccc1-c1nc(NS(=O)(=O)c2cnn(C)c2)nc(Cl)c1C(C)(C)C ; CN1CCN(c2ccc(O)cc2)CC1 ; Cc1ccccc1-c1nc(NS(=O)(=O)c2cnn(C)c2)nc(Oc2ccc(N3CCN(C)CC3)cc2)c1C(C)(C)C]

The compound was prepared in a manner analogous to that described above using 4-(4-methylpiperazin-1-yl)phenol to give N-[5-tert-butyl-4-[4-(4-methylpiperazin-1-yl)phenoxy]-6-(o-tolyl)pyrimidin-2-yl]-1-methyl-pyrazole-4-sulfonamide (hydrochloride salt) (6.8 mg, 30%) as a white solid. ESI-MS m/z calc. 575.2679, found 576.2 (M+1)⁺; Retention time: 1.34 minutes; LC method A.

### Example 462: Preparation of Compound 1498

### Step 1: N-[5-tert-butyl-4-(o-tolyl)-6-phenoxy-pyrimidin-2-yl]-1-methyl-pyrazole-4-sulfonamide

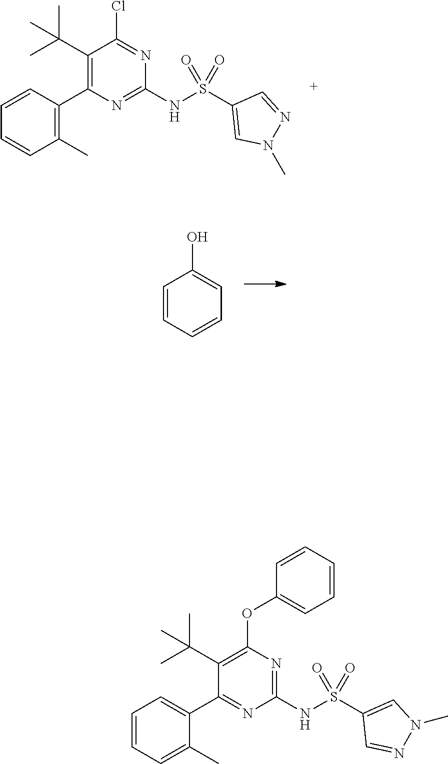 [SMILES: Cc1ccccc1-c1nc(NS(=O)(=O)c2cnn(C)c2)nc(Cl)c1C(C)(C)C ; Oc1ccccc1 ; Cc1ccccc1-c1nc(NS(=O)(=O)c2cnn(C)c2)nc(Oc2ccccc2)c1C(C)(C)C]

The compound was prepared in a manner analogous to that described above using commercially available phenol to give N-[5-tert-butyl-4-(o-tolyl)-6-phenoxy-pyrimidin-2-yl]-1 -methyl-pyrazole-4-sulfonamide (10.5 mg, 55%) as a white solid. ESI-MS m/z calc. 477.18347, found 478.2 (M+1)⁺; Retention time: 1.87 minutes; LC method A.

### Example 463: Preparation of Compound 1499

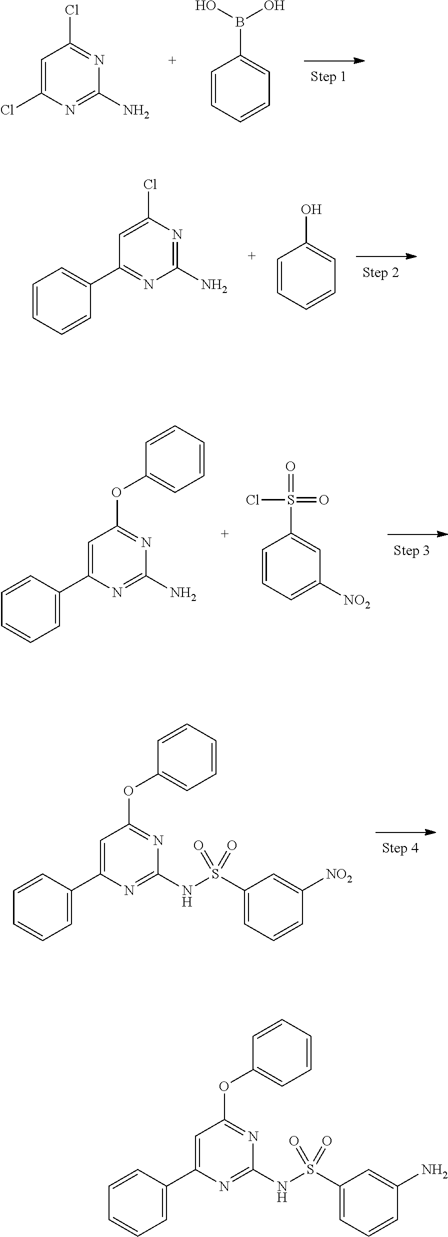

### Step 1: 4-Chloro-6-phenyl-pyrimidin-2-amine

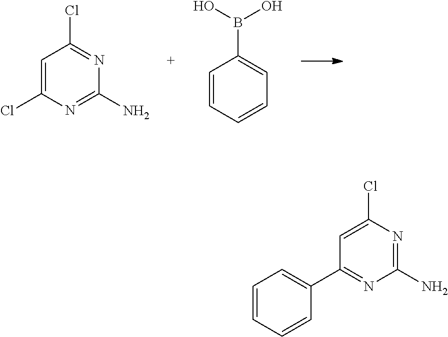 [SMILES: Nc1nc(Cl)cc(Cl)n1 ; OB(O)c1ccccc1 ; Nc1nc(Cl)cc(-c2ccccc2)n1]

A mixture of 4,6-dichloropyrimidin-2-amine (2 g, 12.20 mmol), phenylboronic acid (approximately 743.8 mg, 6.100 mmol), potassium carbonate (approximately 12.20 mL of 2 M, 24.40 mmol) and tetrakis(triphenylphosphine)palladium (0) (approximately 352.4 mg, 0.3050 mmol) in CH₃CN (20 mL) was heated at 90° C. for 4 hours. The reaction mixture was poured into water and extracted with EtOAc (3×). The combined organics were dried over sodium sulfate, and evaporated. Purification by column chromatography (24 g silica; 0-40% ethyl acetate in hexanes) gave 4-chloro-6-phenyl-pyrimidin-2-amine (1.6 g, 64%) as a white solid. ESI-MS m/z calc. 205.04068, found 206.2 (M+1)⁺; Retention time: 0.53 minutes; LC method D.

### Step 2: 4-Phenoxy-6-phenyl-pyrimidin-2-amine

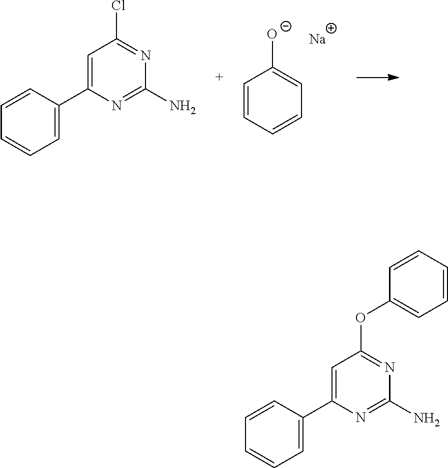 [SMILES: Nc1nc(Cl)cc(-c2ccccc2)n1 ; [O-]c1ccccc1 ; [Na+] ; Nc1nc(Oc2ccccc2)cc(-c2ccccc2)n1]

4-Chloro-6-phenyl-pyrimidin-2-amine (1269 mg, 6.171 mmol) was mixed with sodium phenoxide (approximately 730.7 mg, 6.294 mmol) in DMF (31.72 mL) under nitrogen and the reaction was heated to 110° C. for 5 hours. The mixture was diluted with 100 mL of water and it was extracted with EtOAc (3×50 mL). The combined organics were washed with brine, dried over sodium sulfate and concentrated under reduced pressure. The residue was dissolved in a 1:5 mixture of EtOH:EtOAc (v:v, 6 mL) and purified by silica gel chromatography using a 0-50% gradient of EtOAc in hexanes over 30 minutes. The desired product was obtained as a white solid. 4-Phenoxy-6-phenyl-pyrimidin-2-amine (1.36 g, 84%).

¹H NMR (400 MHz, DMSO) δ 8.07-8.01 (m, 2H), 7.51-7.41 (m, 5H), 7.28-7.18 (m, 3H), 6.71 (s, 1H), 6.69 (s, 2H). ESI-MS m/z calc. 263.11, found 264.2 (M+1)⁺, Retention time: 1.06 minutes (LC method A).

### Step 3: 3-Nitro-N-(4-phenoxy-6-phenyl-pyrimidin-2-yl)benzenesulfonamide

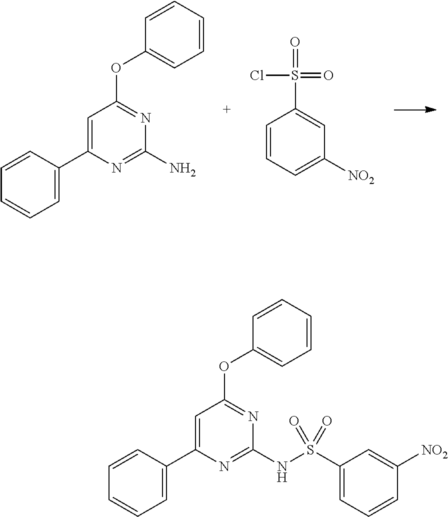 [SMILES: Nc1nc(Oc2ccccc2)cc(-c2ccccc2)n1 ; O=[N+]([O-])c1cccc(S(=O)(=O)Cl)c1 ; O=[N+]([O-])c1cccc(S(=O)(=O)Nc2nc(Oc3ccccc3)cc(-c3ccccc3)n2)c1]

4-Phenoxy-6-phenyl-pyrimidin-2-amine (58 mg, 0.2203 mmol) and NaH (approximately 5.287 mg, 0.2203 mmol) were mixed in DMA (2 mL) at room temperature. The reaction mixture was allowed to react for 15 minutes and 3-nitrobenzenesulfonyl chloride (approximately 48.82 mg, 0.2203 mmol) was added and the reaction mixture was allowed to stir for an additional 60 minutes. The reaction was filtered and subjected to preparative HPLC using 1-99% gradient of ACN in water (0.05% HCl modifier) over 15 minutes. The compound was used for the next step without further purification.

### Step 4: 3-Amino-N-(4-phenoxy-6-phenyl-pyrimidin-2-yl)benzenesulfonamide

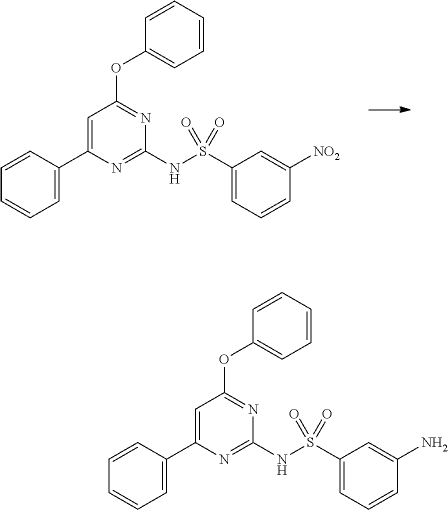 [SMILES: O=[N+]([O-])c1cccc(S(=O)(=O)Nc2nc(Oc3ccccc3)cc(-c3ccccc3)n2)c1 ; Nc1cccc(S(=O)(=O)Nc2nc(Oc3ccccc3)cc(-c3ccccc3)n2)c1]

Iron powder (2 mg, 0.03345 mmol) and HCl (5 μL of 6 M, 0.02900 mmol) were added to a solution of 3-nitro-N-(4-phenoxy-6-phenyl-pyrimidin-2-yl)benzenesulfonamide (15 mg, 0.03345 mmol) in THE (40 μL) and EtOH (20 μL). The mixture was stirred at 95° C. for 30 minutes. The mixture was filtered and subjected to preparative HPLC using a 1-99% gradient of ACN in water (0.05% HCl modifier) over 15 min to give the desired product as white solid. 3-Amino-N-(4-phenoxy-6-phenyl-pyrimidin-2-yl)benzenesulfonamide (5.5 mg, 41%). ¹H NMR (400 MHz, DMSO) δ 11.54 (s, 1H), 8.10-7.90 (m, 2H), 7.54-7.45 (m, 5H), 7.34-7.28 (m, 1H), 7.26-7.21 (m, 2H), 7.18 (s, 1H), 7.10 (t, J=2.1 Hz, 1H), 7.06 (t, J=7.9 Hz, 1H), 6.86 -6.79 (m, 1H), 6.73-6.68 (m, 1H), 5.52 (s, 2H). ESI-MS m/z calc. 418.10995, found 419.3 (M+1)⁺; Retention time: 1.75 minutes (LC method I).

### Example 464: Preparation of Compound 1500

### Step 1: 4-Nitro-N-(4-phenoxy-6-phenyl-pyrimidin-2-yl)benzenesulfonamide

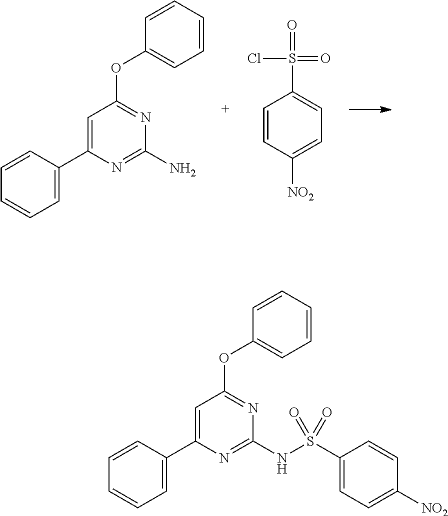 [SMILES: Nc1nc(Oc2ccccc2)cc(-c2ccccc2)n1 ; O=[N+]([O-])c1ccc(S(=O)(=O)Cl)cc1 ; O=[N+]([O-])c1ccc(S(=O)(=O)Nc2nc(Oc3ccccc3)cc(-c3ccccc3)n2)cc1]

4-Phenoxy-6-phenyl-pyrimidin-2-amine (100 mg, 0.3798 mmol) and NaH (approximately 9.114 mg, 0.3798 mmol) were mixed in DMA (3.448 mL) at room temperature. The reaction mixture was allowed to react for 15 minutes and 4-nitrobenzenesulfonyl chloride (approximately 84.17 mg, 0.3798 mmol) was added. The reaction mixture was allowed to stir for 16 hours at 110° C. The reaction was filtered and subjected to preparative HPLC using a 1-99% gradient of ACN in water (0.05% HCl modifier) over 15 minutes. The product was used for the next step without further purification. 4-Nitro-N-(4-phenoxy-6-phenyl-pyrimidin-2-yl)benzenesulfonamide (42.9 mg, 25%). ESI-MS m/z calc. 448.08414, found 449.3 (M+1)⁺; Retention time: 1.85 minutes; LC method A.

### Step 2: 4-Amino-N-(4-phenoxy-6-phenyl-pyrimidin-2-yl)benzenesulfonamide

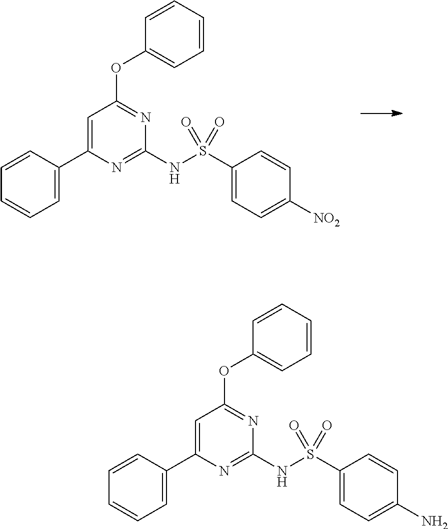 [SMILES: O=[N+]([O-])c1ccc(S(=O)(=O)Nc2nc(Oc3ccccc3)cc(-c3ccccc3)n2)cc1 ; Nc1ccc(S(=O)(=O)Nc2nc(Oc3ccccc3)cc(-c3ccccc3)n2)cc1]

Iron powder (5 mg, 0.09366 mmol) and HCl (10 μL of 6 M, 0.06244 mmol) were added to a solution of 4-nitro-N-(4-phenoxy-6-phenyl-pyrimidin-2-yl)benzenesulfonamide (42 mg, 0.09366 mmol) in THE (100 μL) and EtOH (50 μL). The mixture was stirred at 95° C. for 30 minutes. The mixture was filtered and subjected to preparative HPLC using a 1-99% gradient of ACN in water (0.05% HCl modifier) over 15 min to produce the desired product as white solid. 4-Amino-N-(4-phenoxy-6-phenyl-pyrimidin-2-yl)benzenesulfonamide (10.6 mg, 77%). ¹H NMR (400 MHz, DMSO) δ 11.24 (s, 1H), 8.10-8.00 (m, 2H), 7.60-7.46 (m, 5H), 7.39-7.21 (m, 5H), 7.19 (s, 1H), 6.47-6.38 (m, 2H), 5.96 (s, 2H). ESI-MS m/z calc. 418.10995, found 419.5 (M+1)⁺; Retention time: 1.63 minutes (LC method I).

### Example 465: Preparation of Compound 1501

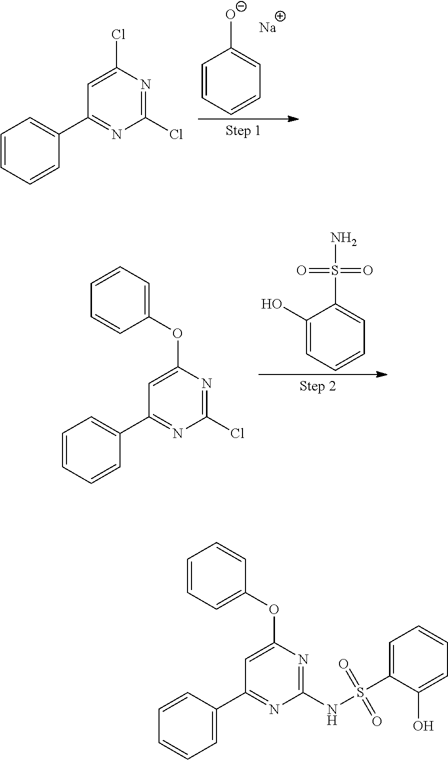 [SMILES: Clc1cc(-c2ccccc2)nc(Cl)n1 ; [O-]c1ccccc1 ; [Na+] ; Clc1nc(Oc2ccccc2)cc(-c2ccccc2)n1 ; NS(=O)(=O)c1ccccc1O ; O=S(=O)(Nc1nc(Oc2ccccc2)cc(-c2ccccc2)n1)c1ccccc1O]

### Step 1: 2-Chloro-4-phenoxy-6-phenyl-pyrimidine

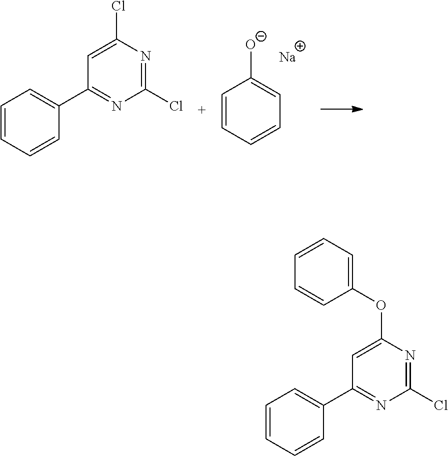 [SMILES: Clc1cc(-c2ccccc2)nc(Cl)n1 ; [O-]c1ccccc1 ; [Na+] ; Clc1nc(Oc2ccccc2)cc(-c2ccccc2)n1]

2,4-Dichloro-6-phenyl-pyrimidine (1.00 g, 4.443 mmol) and sodium phenoxide (515.8 mg, 4.443 mmol) were mixed in THE (5 mL) and the reaction mixture was stirred at room temperature for 15 minutes. The reaction was filtered and evaporated. The residue was dissolved in DCM and washed with brine. The cloudy organic phase was filtered, and the solvent was evaporated. The residue was slurried in a mixture of DCM (1 mL), diethylether (1 mL) and EtOH (2 mL). The solid was filtered to give 2-chloro-4-phenoxy-6-phenyl-pyrimidine (1.200 g, 91%) as a white powder. ¹H NMR (400 MHz, DMSO) δ 8.26-8.13 (m, 2H), 7.79 (s, 1H), 7.65-7.43 (m, 5H), 7.40-7.22 (m, 3H). ESI-MS m/z calc. 282.056, found 283.3 (M+1)⁺, Retention time: 2.86 minutes (LC method I).

### Step 2: 2-Hydroxy-N-(4-phenoxy-6-phenyl-pyrimidin-2-yl)benzenesulfonamide

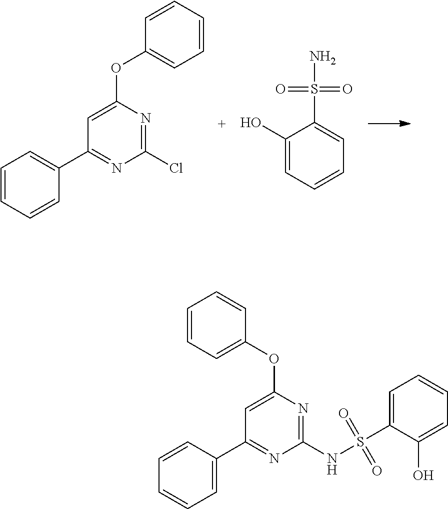 [SMILES: Clc1nc(Oc2ccccc2)cc(-c2ccccc2)n1 ; NS(=O)(=O)c1ccccc1O ; O=S(=O)(Nc1nc(Oc2ccccc2)cc(-c2ccccc2)n1)c1ccccc1O]

2-Hydroxybenzenesulfonamide (approximately 18.38 mg, 0.1061 mmol) was dissolved in DMF (0.5 mL) and NaH (approximately 8.487 mg, 0.2122 mmol) was added and the reaction mixture was purged with nitrogen, capped and stirred at room temperature for 30 minutes. 2-Chloro-4-phenoxy-6-phenyl-pyrimidine (30 mg, 0.1061 mmol) in DMF (1 mL) was added and the reaction mixture was heated to 100° C. for 3 hours. The reaction mixture was filtered and subjected to HPLC purification using a 1-99% gradient over 15 minutes of ACN in water (0.05% HCl modifier) to give 2-hydroxy-N-(4-phenoxy-6-phenyl-pyrimidin-2-yl)benzenesulfonamide (14.7 mg, 31%) as a white solid. ¹H NMR (400 MHz, DMSO) δ 11.53 (s, 1H), 10.58 (s, 1H), 7.98-7.86 (m, 2H), 7.56 (dd, J=8.0, 1.8 Hz, 1H), 7.53-7.43 (m, 5H), 7.41-7.28 (m, 2H), 7.25-7.16 (m, 2H), 7.13 (s, 1H), 6.86 (dd, J=8.2, 1.1 Hz, 1H), 6.84-6.76 (m, 1H). ESI-MS m/z calc. 419.09396, found 420.4 (M+1)⁺; Retention time: 1.82 minutes (LC method A).

### Example 466: Preparation of Compound 1502

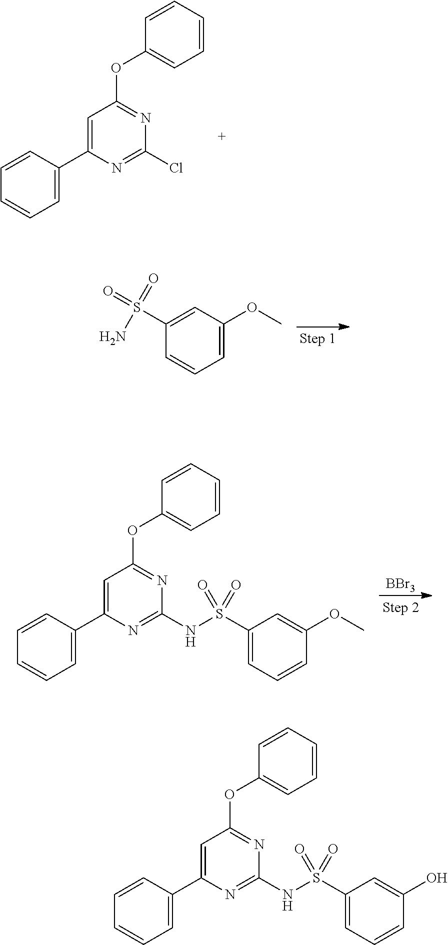 [SMILES: Clc1nc(Oc2ccccc2)cc(-c2ccccc2)n1 ; COc1cccc(S(N)(=O)=O)c1 ; COc1cccc(S(=O)(=O)Nc2nc(Oc3ccccc3)cc(-c3ccccc3)n2)c1 ; "BBr3" ; O=S(=O)(Nc1nc(Oc2ccccc2)cc(-c2ccccc2)n1)c1cccc(O)c1]

### Step 1: 3-Methoxy-N-(4-phenoxy-6-phenyl-pyrimidin-2-yl)benzenesulfonamide

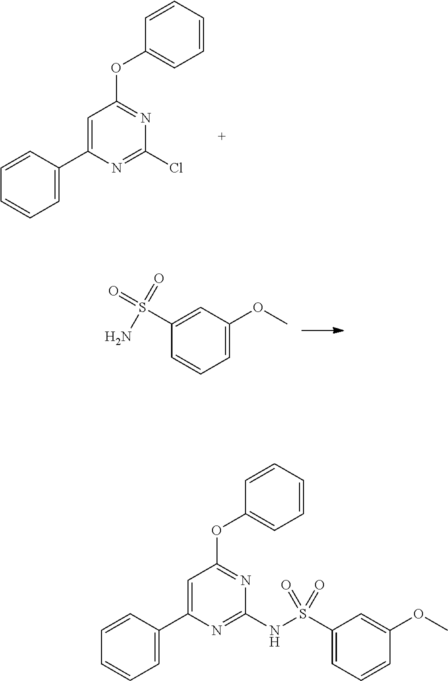 [SMILES: Clc1nc(Oc2ccccc2)cc(-c2ccccc2)n1 ; COc1cccc(S(N)(=O)=O)c1 ; COc1cccc(S(=O)(=O)Nc2nc(Oc3ccccc3)cc(-c3ccccc3)n2)c1]

3-Methoxybenzenesulfonamide (approximately 16.56 mg, 0.08843 mmol) was dissolved in DMF (0.5 mL) and NaH (approximately 7.075 mg, 0.1769 mmol) was added. The mixture was left to react for 10 minutes at room temperature under nitrogen. A solution of 2-chloro-4-phenoxy-6-phenyl-pyrimidine (25 mg, 0.08843 mmol) in DMF (1 mL) was added to the reaction mixture and it was heated at 100° C. for 16 hours. The reaction was filtered and purified by reverse phase HPLC using a 1-99% gradient over 15 minutes of acetonitrile in aqueous 5 mM HCl to give 3-methoxy-N-(4-phenoxy-6-phenyl-pyrimidin-2-yl)benzenesulfonamide (2.4 mg, 6%). ¹H NMR (400 MHz, MeOD) δ 7.92 (t, J=1.6 Hz, 1H), 7.90 (d, J=1.9 Hz, 1H), 7.53-7.41 (m, 6H), 7.35 (t, J=8.0 Hz, 1H), 7.31-7.25 (m, 2H), 7.20 -7.18 (m, 1H), 7.18-7.12 (m, 3H), 3.80 (s, 3H). ESI-MS m/z calc. 433.10962, found 434.2 (M+1)⁺; Retention time: 1.87 minutes; LC method A.

### Step 2: 3-Hydroxy-N-(4-phenoxy-6-phenyl-pyrimidin-2-yl)benzenesulfonamide

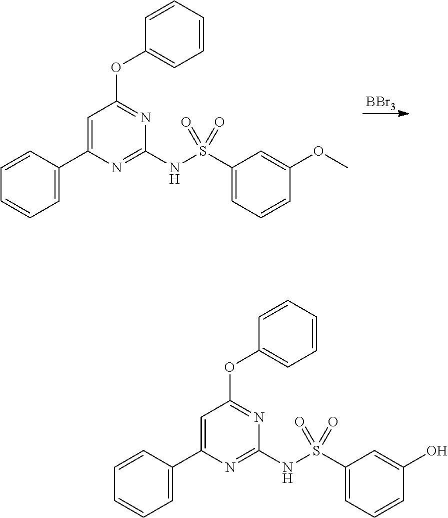 [SMILES: COc1cccc(S(=O)(=O)Nc2nc(Oc3ccccc3)cc(-c3ccccc3)n2)c1 ; "BBr3" ; O=S(=O)(Nc1nc(Oc2ccccc2)cc(-c2ccccc2)n1)c1cccc(O)c1]

3-Methoxy-N-(4-phenoxy-6-phenyl-pyrimidin-2-yl)benzenesulfonamide (20 mg, 0.04614 mmol) was dissolved in dichloromethane (398.8 μL) and cooled down to −78° C. BBr₃ (approximately 366.8 mg of 1 M in DCM, 138.4 μL of 1 M, 0.1384 mmol) was added slowly and the reaction was allowed to slowly warm to room temperature and stir for 4 hours. The mixture was filtered and washed with NaHCO₃(1 mL). The organic layer was separated, filtered and evaporated. The residue was dissolved in MeOH and purified by reverse phase HPLC using a 1-99% gradient over 15 minutes of acetonitrile in aqueous 5 mM HCl to give 3-hydroxy-N-(4-phenoxy-6-phenyl-pyrimidin-2-yl)benzenesulfonamide (15.5 mg, 76%) as a white solid. ¹H NMR (400 MHz, DMSO) δ 11.68 (s, 1H), 10.07 (s, 1H), 8.09-7.92 (m, 2H), 7.55-7.45 (m, 5H), 7.36-7.29 (m, 1H), 7.29-7.26 (m, 1H), 7.25-7.20 (m, 4H), 7.13-7.07 (m, 1H), 6.95 (ddd, J=8.4, 2.4, 1.1 Hz, 1H). ESI-MS m/z calc. 419.09396, found 420.3 (M+1)⁺; Retention time: 2.41 minutes (LC method I).

### Example 467: Preparation of Compound 1503

### Step 1: 6-Nitro-N-(4-phenoxy-6-phenyl-pyrimidin-2-yl)pyridine-3-sulfonamide

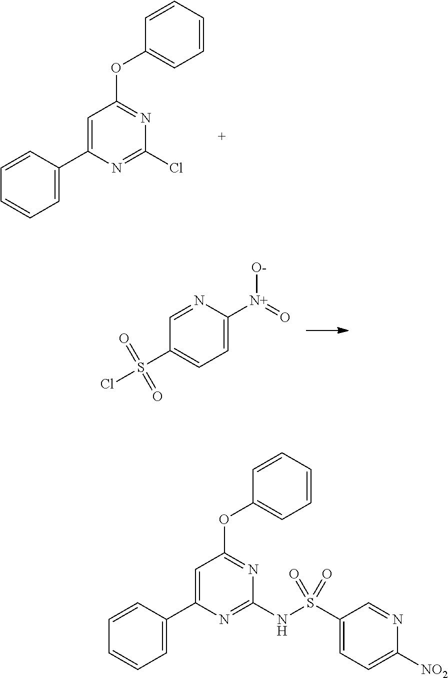 [SMILES: Clc1nc(Oc2ccccc2)cc(-c2ccccc2)n1 ; O=[N+]([O-])c1ccc(S(=O)(=O)Cl)cn1 ; O=[N+]([O-])c1ccc(S(=O)(=O)Nc2nc(Oc3ccccc3)cc(-c3ccccc3)n2)cn1]

A solution of 4-phenoxy-6-phenyl-pyrimidin-2-amine (23 mg, 0.08735 mmol) in NMP (1 mL) was cooled to −20° C. and potassium tert-butoxide (approximately 19.60 mg, 0.1747 mmol) in NMP (500 μL) was added. The reaction mixture was allowed to warm to room temperature over 15-20 minutes. The reaction was cooled to −20° C. and 6-nitropyridine-3-sulfonyl chloride (approximately 38.89 mg, 0.1747 mmol) in NMP (500 μL) was added and the reaction mixture was allowed to warm to room temperature and was stirred for 60 minutes. The reaction mixture was diluted in DMSO and was purified by reverse phase chromatography (1-99% ACN in water (HCl modifier)) to give 6-nitro-N-(4-phenoxy-6-phenyl-pyrimidin-2-yl)pyridine-3-sulfonamide (12 mg, 31%) as a white solid. ESI-MS m/z calc. 449.07938, found 450.32 (M+1)⁺; Retention time: 1.4 minutes; LC method A.

### Step 2: 6-Amino-N-(4-phenoxy-6-phenyl-pyrimidin-2-yl)pyridine-3-sulfonamide

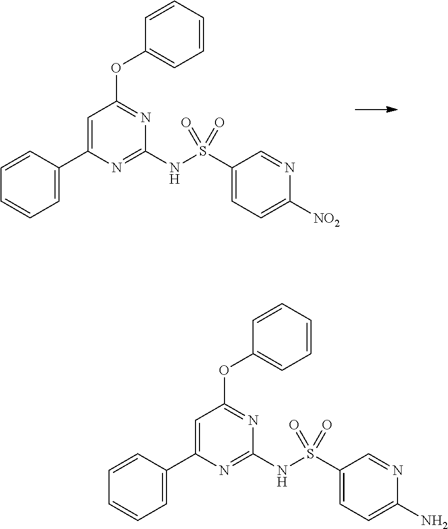 [SMILES: O=[N+]([O-])c1ccc(S(=O)(=O)Nc2nc(Oc3ccccc3)cc(-c3ccccc3)n2)cn1 ; Nc1ccc(S(=O)(=O)Nc2nc(Oc3ccccc3)cc(-c3ccccc3)n2)cn1]

To a solution of 6-nitro-N-(4-phenoxy-6-phenyl-pyrimidin-2-yl)pyridine-3-sulfonamide (approximately 6.000 mg, 0.01335 mmol) in MeOH (4 mL) was added palladium on carbon (approximately 0.2841 mg, 0.002670 mmol). The reaction was purged with hydrogen for 5 minutes and fitted with a hydrogen balloon and stirred at 50° C. for 2 hours. The reaction mixture was filtered and evaporated to give 6-amino-N-(4-phenoxy-6-phenyl-pyrimidin-2-yl)pyridine-3-sulfonamide (5 mg, 89%). ESI-MS m/z calc. 419.10522, found 420.35 (M+1)⁺; Retention time: 1.21 minutes; LC method A.

### Example 468: Preparation of Compound 1504

### Step 1: 6-(4,4-Difluoro-1-piperidyl)-N-(4-phenoxy-6-phenyl-pyrimidin-2-yl)pyridine-2-sulfonamide

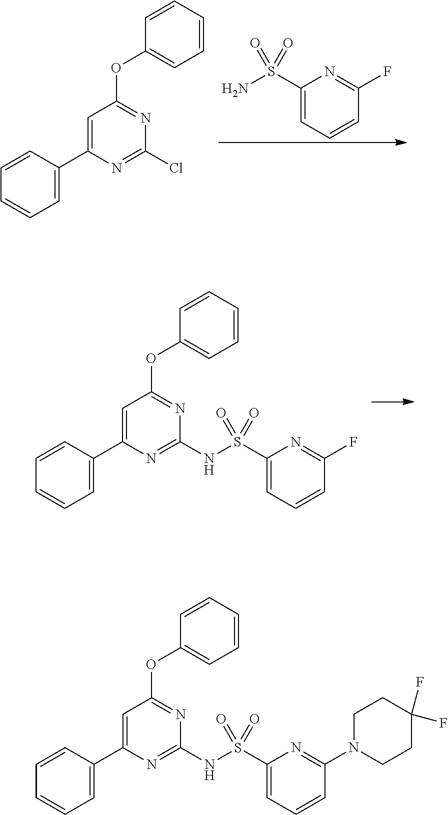 [SMILES: Clc1nc(Oc2ccccc2)cc(-c2ccccc2)n1 ; NS(=O)(=O)c1cccc(F)n1 ; O=S(=O)(Nc1nc(Oc2ccccc2)cc(-c2ccccc2)n1)c1cccc(F)n1 ; O=S(=O)(Nc1nc(Oc2ccccc2)cc(-c2ccccc2)n1)c1cccc(N2CCC(F)(F)CC2)n1]

Stage 1: To a mixture of Pd₂(dba)₃ (155 mg, 0.1693 mmol) and di-tert-butyl-[2-(2,4,6-triisopropylphenyl)phenyl]phosphane (73.6 mg, 0.1733 mmol) in dioxane (2.000 mL) was added 2-chloro-4-phenoxy-6-phenyl-pyrimidine (50 mg, 0.1769 mmol), 6-fluoropyridine-2-sulfonamide (32 mg, 0.1816 mmol) and Cs₂CO₃ (65 mg, 0.1995 mmol). The reaction mixture was stirred at 95° C. for 50 minutes. The reaction mixture was filtered and purified by HPLC (15-75% ACN in water (HCl modifier)) to give 6-fluoro-N-(4-phenoxy-6-phenyl-pyrimidin-2-yl)pyridine-2-sulfonamide (38.6 mg, 51%). ¹H NMR (400 MHz, DMSO-d₆) δ 12.28 (s, 1H), 8.09 (d, J=7.7 Hz, 1H), 7.95 (d, J=8.3 Hz, 2H), 7.55-7.46 (m, 5H), 7.43 (d, J=6.5 Hz, 1H), 7.35 (t, J=7.4 Hz, 1H), 7.26 (s, 1H), 7.17 (d, J=8.6 Hz, 2H). ESI-MS m/z calc. 422.0849, found 423.0 (M+1)⁺; Retention time: 1.7 minutes (LC method A).

Stage 2: A mixture of 6-fluoro-N-(4-phenoxy-6-phenyl-pyrimidin-2-yl)pyridine-2-sulfonamide (38.6 mg, 51%), Cs₂CO₃ (250 mg, 0.7673 mmol), 4,4-difluoropiperidine (hydrochloride salt) (36 mg, 0.2284 mmol), and DMSO (1 mL) was stirred at 130° C. for 16 hours. The reaction mixture was cooled down to room temperature, filtered and purified by reverse phase HPLC (20-80% gradient of ACN in H₂O, HCl modifier) to give 6-(4,4-difluoro-1-piperidyl)-N-(4-phenoxy-6-phenyl-pyrimidin-2-yl)pyridine-2-sulfonamide (5.1 mg, 5%). ESI-MS m/z calc. 523.149, found 524.0 (M+1)⁺; Retention time: 1.96 minutes (3 min run). LC method A.

### Example 469: Preparation of Compound 1505 and Compound 1506

### Step 1: 1-Benzyl-N-(4-phenoxy-6-phenyl-pyrimidin-2-yl)pyrazole-4-sulfonamide

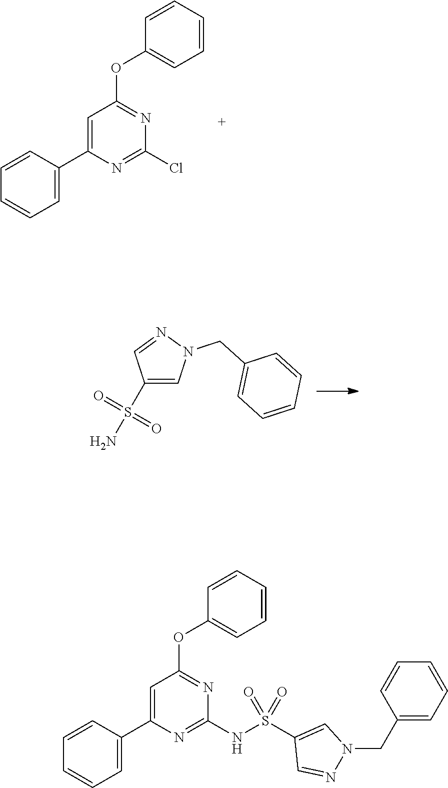 [SMILES: Clc1nc(Oc2ccccc2)cc(-c2ccccc2)n1 ; NS(=O)(=O)c1cnn(Cc2ccccc2)c1 ; O=S(=O)(Nc1nc(Oc2ccccc2)cc(-c2ccccc2)n1)c1cnn(Cc2ccccc2)c1]

Nitrogen was bubbled through a mixture of 1-benzylpyrazole-4-sulfonamide (approximately 62.95 mg, 0.2653 mmol), (5-diphenylphosphanyl-9,9-dimethyl-xanthen-4-yl)-diphenyl-phosphane (approximately 7.672 mg, 0.01326 mmol), diacetoxypalladium (approximately 1.489 mg, 0.006632 mmol), cesium carbonate (approximately 57.64 mg, 0.1769 mmol) and 2-chloro-4-phenoxy-6-phenyl-pyrimidine (25 mg, 0.08843 mmol) in dioxane (1.000 mL) for 5 minutes at room temperature. The reaction mixture was stirred at 100° C. for 1 hour. The mixture was diluted with MeOH, filtered and evaporated. The residue was taken up in MeOH and DMSO, filtered and purified by HPLC (20-80% over 30 minutes gradient of ACN in water (HCl modifier)) to give 1-benzyl-N-(4-phenoxy-6-phenyl-pyrimidin-2-yl)pyrazole-4-sulfonamide (16.9 mg, 40%). ¹H NMR (400 MHz, DMSO) δ 11.63 (s, 1H), 8.18-8.06 (m, 3H), 7.59-7.44 (m, 6H), 7.34-7.20 (m, 7H), 7.14-7.08 (m, 2H), 5.26 (s, 2H). ESI-MS m/z calc. 483.1365, found 484.4 (M+1)⁺; Retention time: 2.69 minutes (LC method I).

### Step 2: N-(4-Phenoxy-6-phenyl-pyrimidin-2-yl)-1H-pyrazole-4-sulfonamide

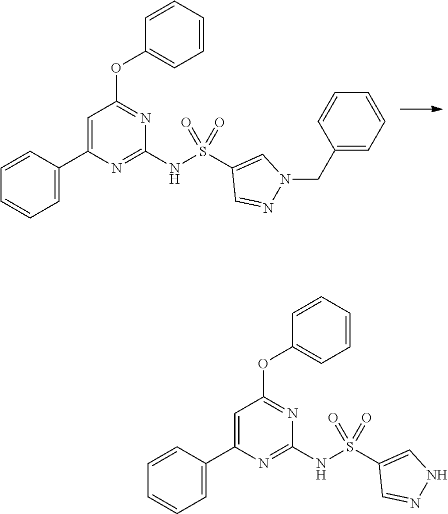 [SMILES: O=S(=O)(Nc1nc(Oc2ccccc2)cc(-c2ccccc2)n1)c1cnn(Cc2ccccc2)c1 ; O=S(=O)(Nc1nc(Oc2ccccc2)cc(-c2ccccc2)n1)c1cn[nH]c1]

1-Benzyl-N-(4-phenoxy-6-phenyl-pyrimidin-2-yl)pyrazole-4-sulfonamide (31 mg, 0.06411 mmol) was dissolved in MeOH (1.5 mL). Dihydroxypalladium (approximately 90.03 mg, 0.6411 mmol) and HCl (approximately 64.11 μL of 1 M, 0.06411 mmol) were added. The mixture was degassed and vigorously stirred under a hydrogen atmosphere for 30 hours. The reaction was filtered and subjected to HPLC using a 1-99% gradient over 15 minutes of ACN in water (0.05% HCl modifier) to produce the desired product as a white powder N-(4-phenoxy-6-phenyl-pyrimidin-2-yl)-1H-pyrazole-4-sulfonamide (hydrochloride salt) (1.7 mg). ¹H NMR (400 MHz, DMSO) δ 11.56 (s, 1H), 8.16-7.99 (m, 2H), 7.67 (s, 2H), 7.59-7.45 (m, 5H), 7.39-7.17 (m, 4H). ESI-MS m/z calc. 393.08957, found 394.3 (M+1)⁺; Retention time: 2.05 minutes (LC method I).

### Example 470: Preparation of Compound 1507

### Step 1: 1-Ethyl-N-(4-phenoxy-6-phenyl-pyrimidin-2-yl)pyrazole-4-sulfonamide

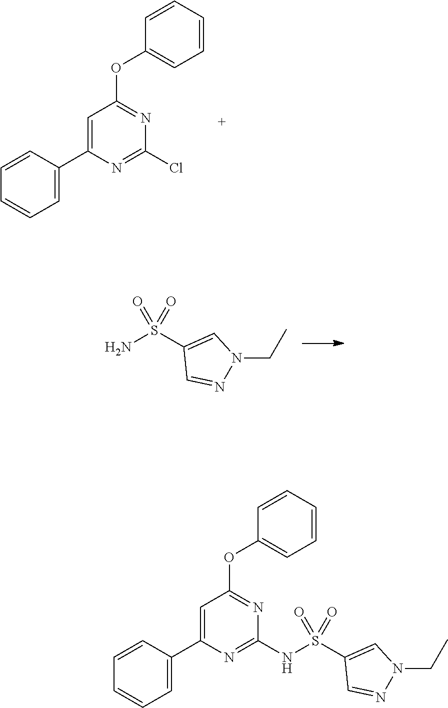 [SMILES: Clc1nc(Oc2ccccc2)cc(-c2ccccc2)n1 ; CCn1cc(S(N)(=O)=O)cn1 ; CCn1cc(S(=O)(=O)Nc2nc(Oc3ccccc3)cc(-c3ccccc3)n2)cn1]

Nitrogen was bubbled through a mixture of 1-ethylpyrazole-4-sulfonamide (approximately 46.48 mg, 0.2653 mmol), (5-diphenylphosphanyl-9,9-dimethyl-xanthen-4-yl)-diphenyl-phosphane (approximately 7.672 mg, 0.01326 mmol), diacetoxypalladium (approximately 1.489 mg, 0.006632 mmol), cesium carbonate (approximately 57.64 mg, 0.1769 mmol) and 2-chloro-4-phenoxy-6-phenyl-pyrimidine (25 mg, 0.08843 mmol) in dioxane (1.000 mL) for 15 minutes at room temperature. The reaction mixture was capped then stirred at 120° C. for 0.5 hours. The reaction mixture was diluted with MeOH, filtered and evaporated. The residue was taken up in MeOH and DMSO, filtered and purified by HPLC (20-80% gradient over 30 minutes of ACN in water (HCL modifier)) to give 1-ethyl-N-(4-phenoxy-6-phenyl-pyrimidin-2-yl)pyrazole-4-sulfonamide (11.2 mg, 30%). ESI-MS m/z calc. 421.12085, found 422.4 (M+1)⁺; Retention time: 2.4 minutes (LC method I).

### Example 471: Preparation of Compound 1508

### Step 1: 3-(dimethylamino)-N-(4-phenoxy-6-phenyl-pyrimidin-2-yl)benzenesulfonamide

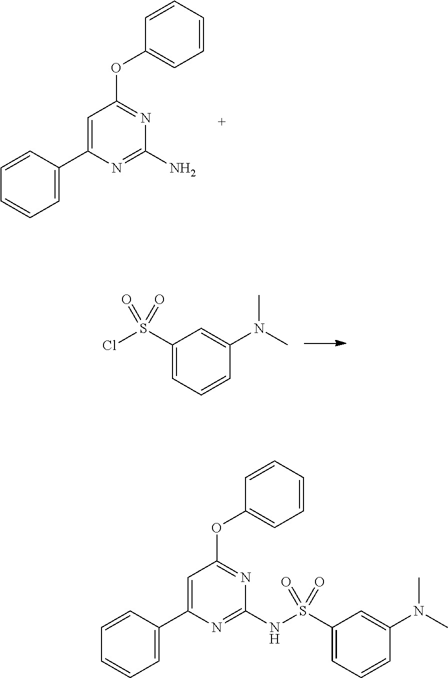 [SMILES: Nc1nc(Oc2ccccc2)cc(-c2ccccc2)n1 ; CN(C)c1cccc(S(=O)(=O)Cl)c1 ; CN(C)c1cccc(S(=O)(=O)Nc2nc(Oc3ccccc3)cc(-c3ccccc3)n2)c1]

To a solution of 4-phenoxy-6-phenyl-pyrimidin-2-amine (20 mg, 0.07596 mmol) in DMF was added NaH (approximately 9.115 mg of 60% w/w, 0.2279 mmol) at 0° C. The reaction was stirred for 5 minutes at 5° C. and it was cooled back to 0° C. To this mixture was added 3-(dimethylamino)benzenesulfonyl chloride (approximately 25.02 mg, 0.1139 mmol) and the reaction was stirred for 10 minutes. The mixture was quenched with 2 drops of water. Filtration and purification by reverse phase HPLC using a 1-99% gradient of acetonitrile in water (HCl modifier) provided 3-(dimethylamino)-N-(4-phenoxy-6-phenyl-pyrimidin-2-yl)benzenesulfonamide (8.2 mg, 24%). ESI-MS m/z calc. 446.14127, found 447.52 (M+1)⁺; Retention time: 1.96 minutes; LC method A. ¹H NMR (400 MHz, Chloroform-d) δ 7.95-7.91 (m, 2H), 7.50-7.38 (m, 6H), 7.34-7.28 (m, 1H), 7.22-7.12 (m, 4H), 6.84 (s, 2H), 2.89 (s, 6H).

### Example 472: Preparation of Compound 1509

### Step 1: 2-Nitro-N-(4-phenoxy-6-phenyl-pyrimidin-2-yl)benzenesulfonamide

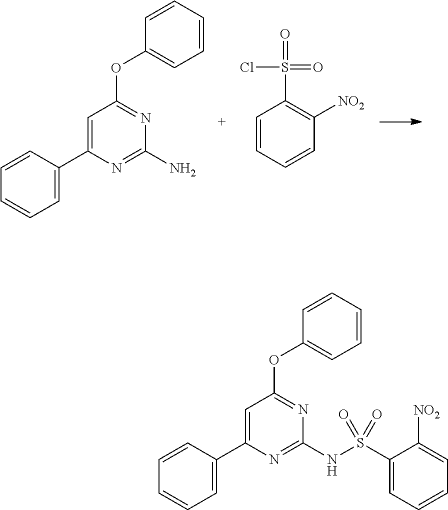 [SMILES: Nc1nc(Oc2ccccc2)cc(-c2ccccc2)n1 ; O=[N+]([O-])c1ccccc1S(=O)(=O)Cl ; O=[N+]([O-])c1ccccc1S(=O)(=O)Nc1nc(Oc2ccccc2)cc(-c2ccccc2)n1]

The compound was prepared in a manner analogous to that described above using commercially available 2-nitrobenzenesulfonyl chloride to give 2-nitro-N-(4-phenoxy-6-phenyl-pyrimidin-2-yl)benzenesulfonamide that was used for the next step.

### Step 2: 2-Amino-N-(4-phenoxy-6-phenyl-pyrimidin-2-yl)benzenesulfonamide

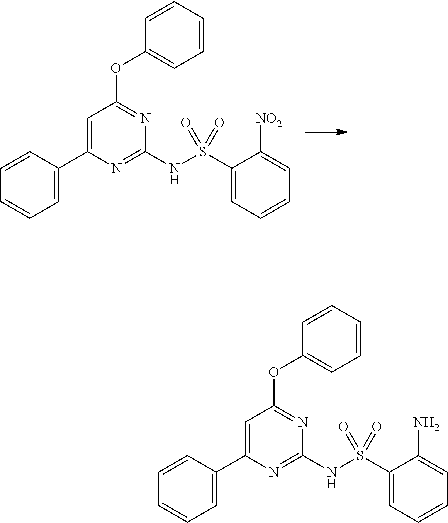 [SMILES: O=[N+]([O-])c1ccccc1S(=O)(=O)Nc1nc(Oc2ccccc2)cc(-c2ccccc2)n1 ; Nc1ccccc1S(=O)(=O)Nc1nc(Oc2ccccc2)cc(-c2ccccc2)n1]

Iron powder (4 mg, 0.07136 mmol) and HCl (8 μL of 6 M, 0.04757 mmol) were added to a solution of 2-nitro-N-(4-phenoxy-6-phenyl-pyrimidin-2-yl)benzenesulfonamide (32 mg, 0.07136 mmol) in THE (80μL) and EtOH (40 μL). The mixture was stirred at 95° C. for 30minutes. It was filtered and subjected to HPLC purification using a 1-99% gradient over 15 minutes of ACN in water (0.05% HCl modifier). After evaporation the desired product was isolated as a white solid. 2-Amino-N-(4-phenoxy-6-phenyl-pyrimidin-2-yl)benzenesulfonamide (10.5 mg). ¹H NMR (400 MHz, DMSO) δ 11.61-11.32 (m, 1H), 8.06 (dq, J=7.8, 2.5, 2.0 Hz, 2H), 7.59-7.48 (m, 5H), 7.41-7.31 (m, 2H), 7.26-7.14 (m, 4H), 6.69 (dd, J=8.3, 1.1 Hz, 1H), 6.47-6.37 (m, 1H), 5.97 (s, 2H). ESI-MS m/z calc. 418.10995, found 419.5 (M+1)⁺; Retention time: 1.8 minutes; LC method I.

### Example 473: Preparation of Compound 1510

### Step 1: 6-Fluoro-N-(4-phenoxy-6-phenyl-pyrimidin-2-yl)pyridine-2-sulfonamide

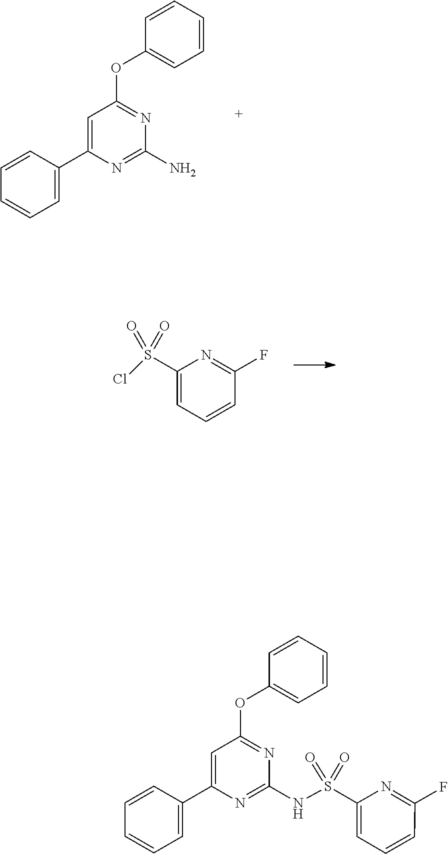 [SMILES: Nc1nc(Oc2ccccc2)cc(-c2ccccc2)n1 ; O=S(=O)(Cl)c1cccc(F)n1 ; O=S(=O)(Nc1nc(Oc2ccccc2)cc(-c2ccccc2)n1)c1cccc(F)n1]

4-Phenoxy-6-phenyl-pyrimidin-2-amine (100 mg, 0.3798 mmol) was dissolved in pyridine, and the mixture was stirred for 5 min and then 6-fluoropyridine-2-sulfonyl chloride (approximately 111.4 mg, 0.5697 mmol) was added. The reaction was stirred at 85° C. for 30 minutes. It was filtered and diluted with DMF. The solution was subjected to HPLC purification using a 1-99% gradient over 15 minutes of ACN in water (0.05% HCl modifier) to give 6-Fluoro-N-(4-phenoxy-6-phenyl-pyrimidin-2-yl)pyridine-2-sulfonamide (60 mg, 37%) as a white powder.

### Step 2: 6-Amino-N-(4-phenoxy-6-phenyl-pyrimidin-2-yl)pyridine-2-sulfonamide

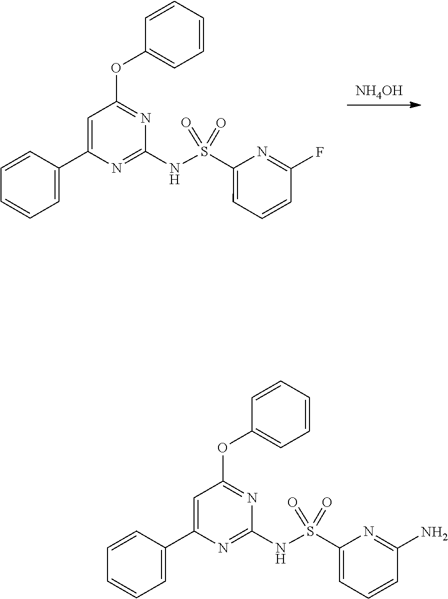 [SMILES: O=S(=O)(Nc1nc(Oc2ccccc2)cc(-c2ccccc2)n1)c1cccc(F)n1 ; "NH4OH" ; Nc1cccc(S(=O)(=O)Nc2nc(Oc3ccccc3)cc(-c3ccccc3)n2)n1]

6-Fluoro-N-(4-phenoxy-6-phenyl-pyrimidin-2-yl)pyridine-2-sulfonamide (30 mg, 0.07102 mmol) was reacted with NH₄₀H (approximately 444.5 mg, 493.9 μL, 3.551 mmol) in a tight sealed tube for 16 hours at 120° C. The reaction was diluted with methanol and purified by HPLC using a 1-99% gradient over 15 minutes of ACN in water (0.05% HCl modifier) to give 6-amino-N-(4-phenoxy-6-phenyl-pyrimidin-2-yl)pyridine-2-sulfonamide (2.7 mg). ¹H NMR (400 MHz, DMSO) δ 11.62 (s, 1H), 7.96 (dd, J=6.6, 1.8 Hz, 2H), 7.54-7.42 (m, 7H), 7.22-7.18(m, 3H), 6.89 (d, J=7.3 Hz, 1H), 6.56 (d, J=8.2 Hz, 1H), 6.41 (s, 2H). ESI-MS m/z calc. 419.10522, found 420.5 (M+1)⁺; Retention time: 2.13 minutes (LC method I).

### Example 474: Preparation of Compound 1511

### Step 1: N-(4-phenoxy-6-phenyl-pyrimidin-2-yl)pyrrolidine-1-sulfonamide

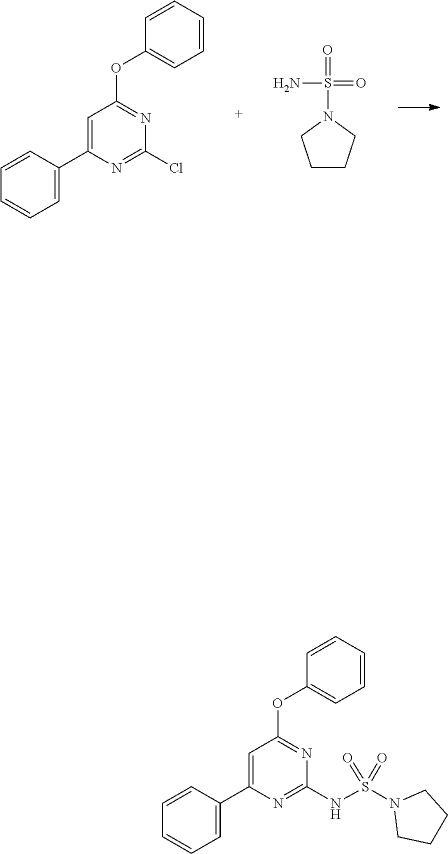 [SMILES: Clc1nc(Oc2ccccc2)cc(-c2ccccc2)n1 ; NS(=O)(=O)N1CCCC1 ; O=S(=O)(Nc1nc(Oc2ccccc2)cc(-c2ccccc2)n1)N1CCCC1]

Nitrogen was bubbled through a mixture of 2-chloro-4-phenoxy-6-phenyl-pyrimidine (25 mg, 0.08843 mmol), pyrrolidine-1-sulfonamide (approximately 39.85 mg, 0.2653 mmol), (5-diphenylphosphanyl-9,9-dimethyl-xanthen-4-yl)-diphenyl-phosphane (approximately 7.672 mg, 0.01326 mmol), diacetoxypalladium (approximately 1.489 mg, 0.006632 mmol) and cesium carbonate (approximately 57.64 mg, 0.1769 mmol) in dioxane (625.0 μL) for 5 min at room temperature. The reaction mixture was capped and stirred at 100° C. for 16 hours. The reaction mixture was diluted with MeOH, filtered and evaporated. The residue was taken up in MeOH and DMSO and purified by HPLC (1-99% ACN in water (HCl modifier)) to give N-(4-phenoxy-6-phenyl-pyrimidin-2-yl)pyrrolidine-1-sulfonamide as a white solid. ¹H NMR (400 MHz, DMSO) δ 10.93 (s, 1H), 8.10-8.13 (m, 2H), 7.63-7.38 (m, 5H), 7.38-7.15 (m, 4H), 3.17-3.02 (m, 4H), 1.64-1.51 (m, 4H). ESI-MS m/z calc. 396.1256, found 397.5 (M+1)⁺; Retention time: 2.67 minutes (LC method I).

### Example 475: Preparation of Compound 1512

### Step 1: 1,5-Dimethyl-N-(4-phenoxy-6-phenyl-pyrimidin-2-yl)pyrazole-4-sulfonamide

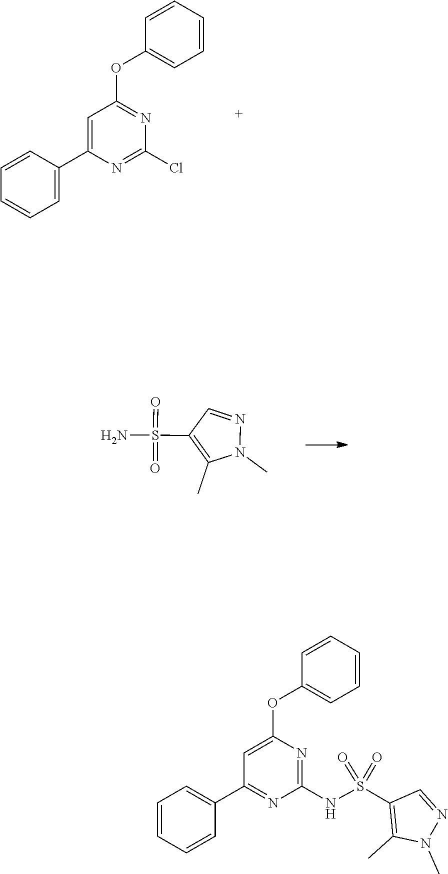 [SMILES: Clc1nc(Oc2ccccc2)cc(-c2ccccc2)n1 ; Cc1c(S(N)(=O)=O)cnn1C ; Cc1c(S(=O)(=O)Nc2nc(Oc3ccccc3)cc(-c3ccccc3)n2)cnn1C]

To a mixture of Pd₂(dba)₃ (8.21 mg, 0.008966 mmol) and di-tert-butyl-[2-(2,4,6-triisopropylphenyl)phenyl]phosphane (6.30 mg, 0.01484 mmol) in a vial was added dioxane (1.5 mL) and the mixture was sparged with nitrogen for 10 minutes. 2-Chloro-4-phenoxy-6-phenyl-pyrimidine (25.5 mg, 0.09019 mmol), 1,5-dimethylpyrazole-4-sulfonamide (approximately 47.41 mg, 0.2706 mmol) and Cs₂CO₃ (approximately 146.9 mg, 0.4509 mmol) were added. The reaction was stirred in a microwave oven at 100° C. for 30 minutes. The mixture was diluted with and purified by HPLC (1-99% ACN in water (HCl modifier)) to give 1,5-dimethyl-N-(4-phenoxy-6-phenyl-pyrimidin-2-yl)pyrazole-4-sulfonamide (12.7 mg, 33%) as a white solid. ESI-MS m/z calc. 421.12085, found 422.31 (M+1)⁺; Retention time: 1.39 minutes; LC method A.

### Example 476: Preparation of Compound 1513

### Step 1: 1-Methyl-N-(4-phenoxy-6-phenyl-pyrimidin-2-yl)pyrazole-3-sulfonamide

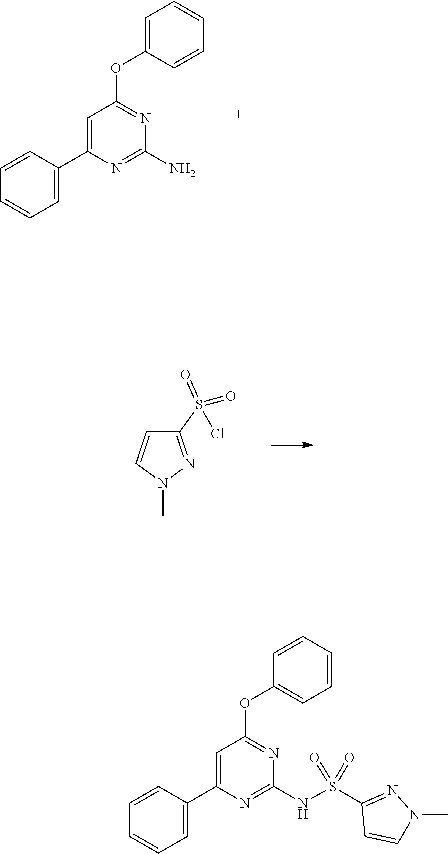

To a solution of 4-phenoxy-6-phenyl-pyrimidin-2-amine (25 mg, 0.09495 mmol) in DMF (1 mL) was added NaH (approximately 11.39 mg, 0.2848 mmol) at 0° C. The reaction was stirred at 5° C. for 5 minutes and it was cooled back to 0° C. To this mixture was added -methylpyrazole-3-sulfonyl chloride (approximately 25.72 mg, 0.1424 mmol) and it was stirred for 16 hours at 100° C. The mixture was filtered and subjected to preparative HPLC purification using a 20-80% gradient over 15 minutes of ACN in water (0.05% HCl modifier) to give 1 -methyl-N-(4-phenoxy-6-phenyl-pyrimidin-2-yl)pyrazole-3-sulfonamide (5.1 mg, 13%). ¹H NMR (400 MHz, DMSO) δ 11.78 (s, 1H), 8.12-8.03 (m, 2H), 7.73 (d, J=2.3 Hz, 1H), 7.57-7.44 (m, 5H), 7.35-7.29 (m, 1H), 7.26-7.20 (m, 3H), 6.35 (s, 1H), 3.86 (s, 3H). ESI-MS m z calc. 407.10522, found 408.3 (M+1)⁺; Retention time: 2.23 minutes (LC method I).

### Example 477: Preparation of Compound 1514

### Step 1: 1,3-Dimethyl-N-(4-phenoxy-6-phenyl-pyrimidin-2-yl)pyrazole-4-sulfonamide

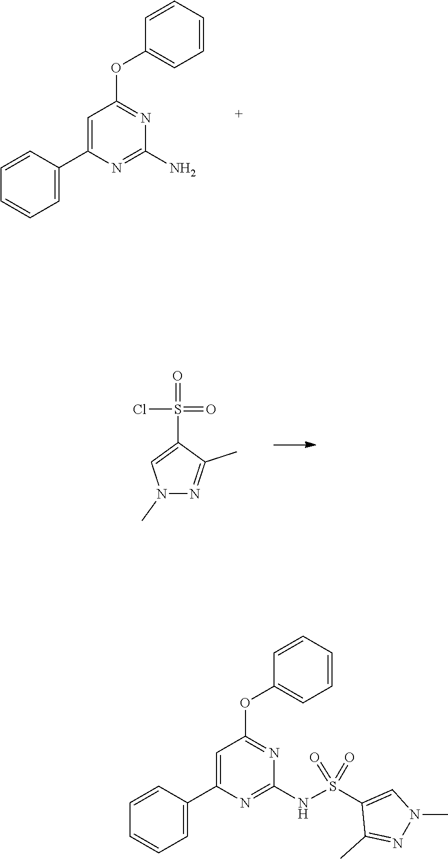

4-Phenoxy-6-phenyl-pyrimidin-2-amine (25 mg, 0.09495 mmol) was dissolved in DMF (1 mL) at 0° C. and sodium hydride (approximately 2.279 mg, 0.09495 mmol) was added. The mixture was allowed to stir for 15 minutes. 1,3-Dimethylpyrazole-4-sulfonyl chloride (approximately 36.96 mg, 0.1899 mmol) was added in two portions and the mixture was allowed to stir for 1 hour at room temperature, then 140° C. for 1 hours. The reaction mixture was filtered and subjected to HPLC using a 1-99% gradient over 15 minutes of ACN in water (0.05% HCl modifier) to give the desired product as a light solid. 1,3-Dimethyl-N-(4-phenoxy-6-phenyl-pyrimidin-2-yl)pyrazole-4-sulfonamide ¹H NMR (400 MHz, DMSO) δ 11.62 (s, 1H), 8.09-8.03 (m, 2H), 7.56-7.50 (m, 5H), 7.43 (s, 1H), 7.38-7.33 (m, 1H), 7.30-7.25 (m, 2H), 7.22 (s, 1H), 3.65 (s, 3H), 2.20 (s, 3H). ESI-MS m/z calc. 421.12085, found 422.3 (M+1)⁺; Retention time: 2.32 minutes (LC method I).

### Example 478: Preparation of Compound 1515

### Step 1: N-[4-(2-fluorophenoxy)-6-(2-isopropylphenyl)pyrimidin-2-yl]thiophene-3-sulfonamide

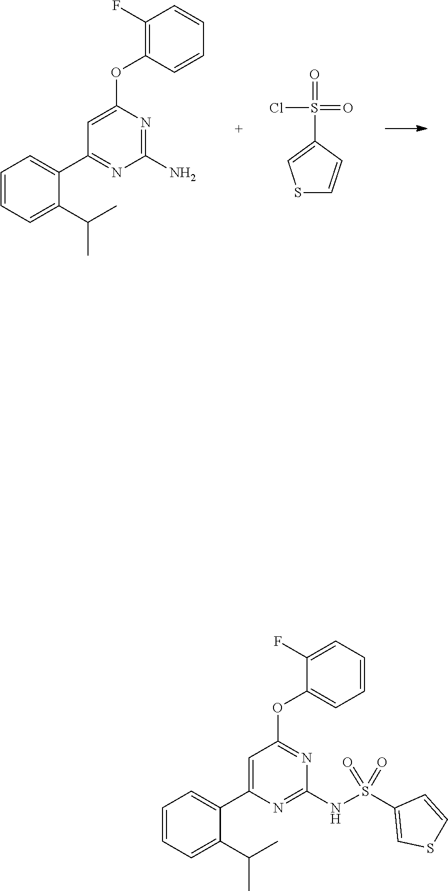 [SMILES: CC(C)c1ccccc1-c1cc(Oc2ccccc2F)nc(N)n1 ; O=S(=O)(Cl)c1ccsc1 ; CC(C)c1ccccc1-c1cc(Oc2ccccc2F)nc(NS(=O)(=O)c2ccsc2)n1]

In a 3-mL vial, 4-(2-fluorophenoxy)-6-(2-isopropylphenyl)pyrimidin-2-amine (25.00 mg, 0.0773 mmol) (25.0 mg, 0.0773 mmol) and thiophene-3-sulfonyl chloride (27.40 mg, 0.15 mmol) were dissolved in pyridine (800 μL). This mixture was heated at 90° C. for 24 hours, after which it was cooled to room temperature and diluted with ethyl acetate (1 mL). This solution was washed with 1 N HCl (3×1 mL), filtered, and purified by reverse phase HPLC (1-99% acetonitrile in water using HCl as a modifier) to give N-[4-(2-fluorophenoxy)-6-(2-isopropylphenyl)pyrimidin-2-yl]thiophene-3-sulfonamide (10.1 mg, 28%). ¹H NMR (400 MHz, dimethylsulfoxide-d₆) δ 11.81 (s, 1H, D20 exchangeable), 7.62-7.34 (m, 8H), 7.32-7.24 (m, 2H), 6.86 (s, 1H), 6.84 (s, 1H), 3.25-3.01 (m, 1H), 1.15 (d, J=6.8 Hz, 6H). ESI-MS m/z calc. 469.09302, found 470.1 (M+1)⁺; Retention time: 2.03 minutes; LC method A.

### Example 479: Preparation of Compound 1516 and Compound 1517

### Step 1: N-[4-(2-fluorophenoxy)-6-(2-isopropylphenyl)pyrimidin-2-yl]-3-methoxy-benzenesulfonamide

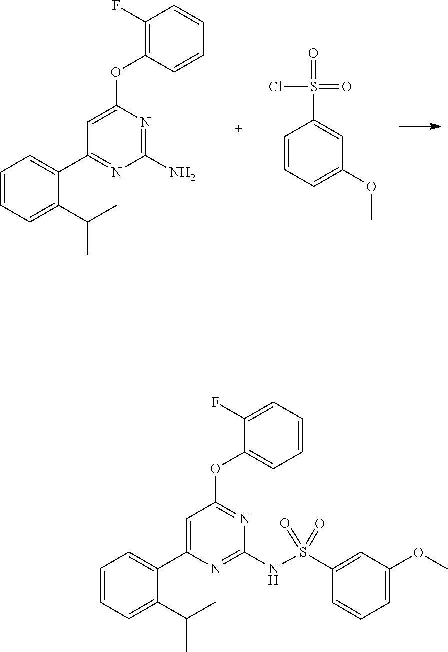 [SMILES: CC(C)c1ccccc1-c1cc(Oc2ccccc2F)nc(N)n1 ; COc1cccc(S(=O)(=O)Cl)c1 ; COc1cccc(S(=O)(=O)Nc2nc(Oc3ccccc3F)cc(-c3ccccc3C(C)C)n2)c1]

In a 3-mL vial, 4-(2-fluorophenoxy)-6-(2-isopropylphenyl)pyrimidin-2-amine (25.00 mg, 0.0773 mmol) (25.0 mg, 0.0773 mmol) and 3-methoxybenzenesulfonyl chloride (31.00 mg, 21.23 μL, 0.15 mmol) were dissolved in pyridine (800 μL). This mixture was heated at 90° C. for 24 h, after which it was cooled to room temperature and diluted with ethyl acetate (1 mL). This solution was washed with 1 N HCl (3×1 mL), filtered, and purified by reverse phase HPLC (1-99% acetonitrile in water using HCl as a modifier) to give N-[4-(2-fluorophenoxy)-6-(2-isopropylphenyl)pyrimidin-2-yl]-3-methoxy-benzenesulfonamide (15.0 mg, 39%). ESI-MS m z calc. 493.14716, found 494.2 (M+1)⁺; Retention time: 2.1 minutes; LC method A.

### Step 2: N-[4-(2-fluorophenoxy)-6-(2-isopropylphenyl)pyrimidin-2-yl]-3-hydroxy-benzenesulfonamide

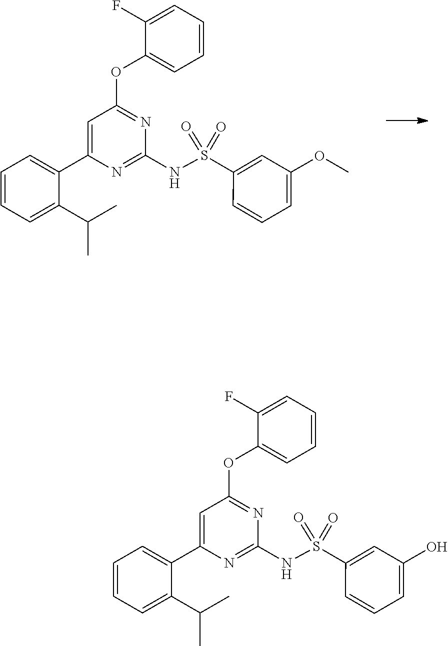 [SMILES: COc1cccc(S(=O)(=O)Nc2nc(Oc3ccccc3F)cc(-c3ccccc3C(C)C)n2)c1 ; CC(C)c1ccccc1-c1cc(Oc2ccccc2F)nc(NS(=O)(=O)c2cccc(O)c2)n1]

In a 3 mL vial with a pressure-relief cap, N-[4-(2-fluorophenoxy)-6-(2-isopropylphenyl)pyrimidin-2-yl]-3-methoxy-benzenesulfonamide (9.0 mg, 0.018 mmol) was dissolved in DCM (400 μL). This solution was cooled to 0° C., then a solution of BBr₃ (100 μL of 1.0 M, 0.1000 mmol) in DCM was added. This solution was stirred at 0° C. for 30 minutes, after which it was quenched with methanol (500 μL), filtered, and purified by reverse phase HPLC (1-99% acetonitrile in water using HCl as a modifier) to give N-[4-(2-fluorophenoxy)-6-(2-isopropylphenyl)pyrimidin-2-yl]-3-hydroxy-benzenesulfonamide (6.2 mg, 72%). ESI-MS m z calc. 479.1315, found 480.2 (M+1)⁺; Retention time: 1.88 minutes; LC method A.

### Example 480: Preparation of Compound 1518

### Step 1: 4-Amino-N-[4-(2-fluorophenoxy)-6-(2-isopropylphenyl)pyrimidin-2-yl]benzenesulfonamide

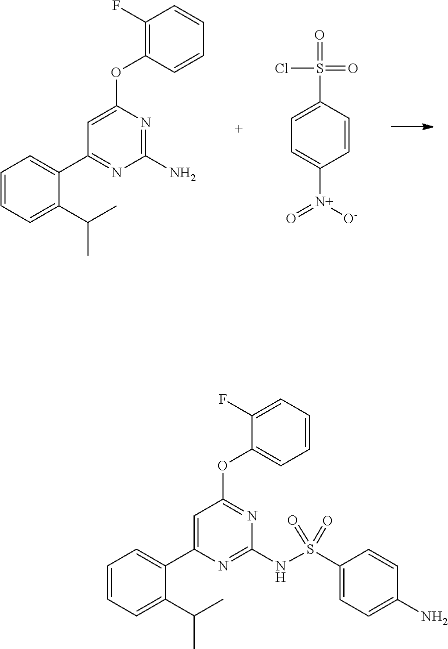 [SMILES: CC(C)c1ccccc1-c1cc(Oc2ccccc2F)nc(N)n1 ; O=[N+]([O-])c1ccc(S(=O)(=O)Cl)cc1 ; CC(C)c1ccccc1-c1cc(Oc2ccccc2F)nc(NS(=O)(=O)c2ccc(N)cc2)n1]

In a 3-mL vial, 4-(2-fluorophenoxy)-6-(2-isopropylphenyl)pyrimidin-2-amine (25.00 mg, 0.0773 mmol) (25.0 mg, 0.0773 mmol) and 4-nitrobenzenesulfonyl chloride (33.24 mg, 0.15 mmol) were dissolved in pyridine (800 μL). This mixture was heated at 90° C. for 24 hours, after which it was cooled to room temperature and diluted with ethyl acetate (1 mL). This solution was washed with 1 N HCl (3×1 mL), filtered, and purified by reverse phase HPLC (1-99% acetonitrile in water using HCl as a modifier) to give a pure nitro product. To a 10-mL microwave vial, the purified nitro product was dissolved in 1:1 EtOH:EtOAc (2.0 mL) and this solution was sparged with a balloon of hydrogen gas for 5 minutes. The cap was briefly removed, and 10% Pd(OH)₂/C (5.4 mg, 0.0038 mmol) was added. This reaction mixture was stirred under H₂ (2 L, 80 mmol) at 60° C. for 2 hours. This mixture was cooled to room temperature, filtered through Celite and rinsed with MeOH (5 mL). Concentration in vacuo, re-dissolution in 1:1 MeOH:DMSO (1.0 mL), filtration and purification by reverse phase HPLC (1-99% acetonitrile in water using HCl as a modifier) gave 4-amino-N-[4-(2-fluorophenoxy)-6-(2-isopropylphenyl)pyrimidin-2-yl]benzenesulfonamide (hydrochloride salt) (7.0 mg, 18%). ESI-MS m/z calc. 478.1475, found 479.2 (M+1)⁺; Retention time: 1.87 minutes; LC method A.

### Example 481: Preparation of Compound 1519

### Step 1: N-[4-(2-fluorophenoxy)-6-(2-isopropylphenyl)pyrimidin-2-yl]pyridine-3-sulfonamide

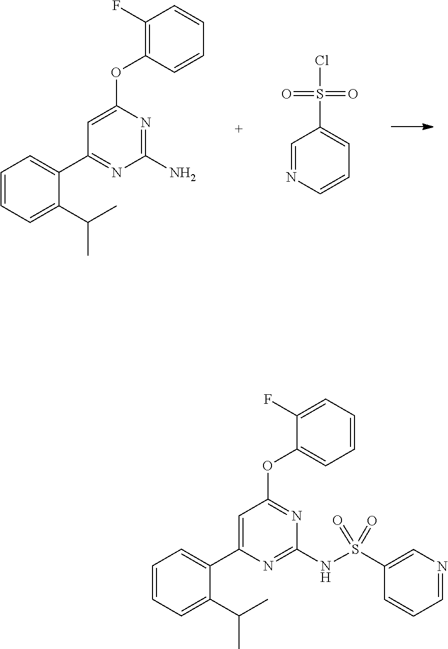 [SMILES: CC(C)c1ccccc1-c1cc(Oc2ccccc2F)nc(N)n1 ; O=S(=O)(Cl)c1cccnc1 ; CC(C)c1ccccc1-c1cc(Oc2ccccc2F)nc(NS(=O)(=O)c2cccnc2)n1]

In a 3-mL vial, 4-(2-fluorophenoxy)-6-(2-isopropylphenyl)pyrimidin-2-amine (25.00 mg, 0.0773 mmol) (25.0 mg, 0.0773 mmol) and pyridine-3-sulfonyl chloride (26.64 mg, 0.15 mmol) were dissolved in pyridine (800 μL). This mixture was heated at 90° C. for 24 hour, after which it was cooled to room temperature and diluted with ethyl acetate (1 mL). This solution was washed with 1 N HCl (3×1 mL), filtered, and purified by reverse phase HPLC (1-99% acetonitrile in water using HCl as a modifier) to give N-[4-(2-fluorophenoxy)-6-(2-isopropylphenyl)pyrimidin-2-yl]pyridine-3-sulfonamide (12.5 mg, 35%). ¹H NMR (400 MHz, dimethylsulfoxide-d₆) δ 12.95-11.65 (bs, 1H, D20 exchangeable), 8.73 (dd, J=4.8, 1.6 Hz, 1H), 8.59 (d, J=2.3 Hz, 1H), 7.66-7.54 (m, 1H), 7.54-7.34 (m, 7H), 7.33-7.22 (m, 2H), 6.83 (s, 1H), 3.15-3.02 (m, 1H), 1.14 (d, J=6.8 Hz, 6H). ESI-MS m/z calc. 464.13184, found 465.2 (M+1)⁺; Retention time: 1.74 minutes; LC method A.

### Example 482: Preparation of Compound 1520 and Compound 1521

### Step 1: N-[4-(2-fluorophenoxy)-6-(2-isopropylphenyl)pyrimidin-2-yl]-4-methoxy-benzenesulfonamide (Compound 1520)

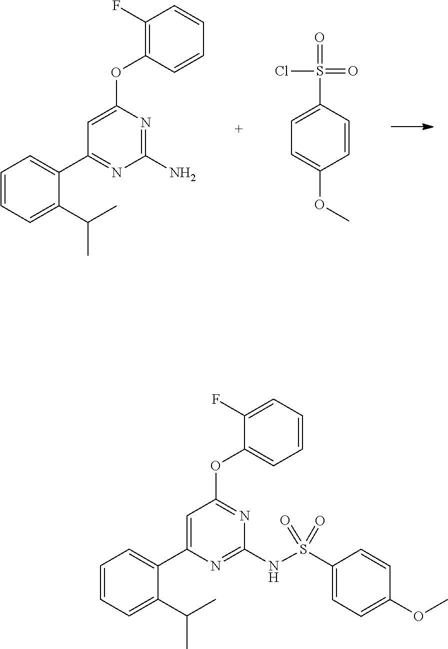 [SMILES: CC(C)c1ccccc1-c1cc(Oc2ccccc2F)nc(N)n1 ; COc1ccc(S(=O)(=O)Cl)cc1 ; COc1ccc(S(=O)(=O)Nc2nc(Oc3ccccc3F)cc(-c3ccccc3C(C)C)n2)cc1]

In a 3-mL vial, 4-(2-fluorophenoxy)-6-(2-isopropylphenyl)pyrimidin-2-amine (25.0 mg, 0.0773 mmol) was dissolved in NMP (800 μL), to which 60% NaH (8.0 mg, 0.20 mmol) was added. This slurry was stirred for 5 minutes at room temperature, after which 4-methoxybenzenesulfonyl chloride (31.00 mg, 0.15 mmol) was added. After 2 hours at room temperature, the reaction was quenched dropwise with 1 N HCl (1 mL), and ethyl acetate (1 mL) was added. After the layers had separated, the organic layer was washed with water (1 mL). Filtration and purification by reverse phase HPLC (1-99% acetonitrile in water using HCl as a modifier) gave N-[4-(2-fluorophenoxy)-6-(2-isopropylphenyl)pyrimidin-2-yl]-4-methoxy-benzenesulfonamide (14.0 mg, 37%). ¹H NMR (400 MHz, dimethylsulfoxide-d₆) δ 11.66 (s, 1H, D20 exchangeable), 7.58-7.40 (m, 6H), 7.37-7.18 (m, 4H), 6.88 (d, J=8.5 Hz, 2H), 6.81 (s, 1H), 3.82 (s, 3H), 3.20-3.03 (m, 1H), 1.13 (d, J=6.9 Hz, 6H). ESI-MS m/z calc. 493.14716, found 494.2 (M+1)⁺; Retention time: 2.09 minutes; LC method A.

### Step 2: N-[4-(2-fluorophenoxy)-6-(2-isopropylphenyl)pyrimidin-2-yl]-4-hydroxy-benzenesulfonamide (Compound 1521)

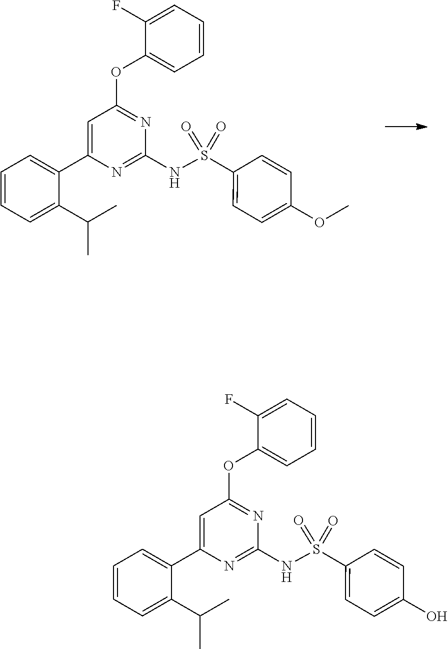 [SMILES: COc1ccc(S(=O)(=O)Nc2nc(Oc3ccccc3F)cc(-c3ccccc3C(C)C)n2)cc1 ; CC(C)c1ccccc1-c1cc(Oc2ccccc2F)nc(NS(=O)(=O)c2ccc(O)cc2)n1]

In a 3 mL vial with a pressure-relief cap, N-[4-(2-fluorophenoxy)-6-(2-isopropylphenyl)pyrimidin-2-yl]-4-methoxy-benzenesulfonamide (9.0 mg, 0.01824 mmol), MeCN (500 μL) and TMS-I (50 μL, 0.35 mmol) were added. This solution was heated to 100° C. for 16 hours, after which it was cooled to room temperature. Another portion of TMS-I (50 μL, 0.35 mmol) was added, and this mixture was heated to 100° C. for 4 hours. After cooling to room temperature, the mixture was diluted with dichloromethane (1 mL) and washed with saturated sodium thiosulfate solution (2×1 mL), water (1 mL) and saturated sodium chloride solution (1 mL). This solution was filtered and purified by reverse phase HPLC (1-99% acetonitrile in water using HCl as a modifier) to give N-[4-(2-fluorophenoxy)-6-(2-isopropylphenyl)pyrimidin-2-yl]-4-hydroxy-benzenesulfonamide (2.4 mg, 27%). ESI-MS m/z calc. 479.1315, found 480.2 (M+1)⁺; Retention time: 1.83 minutes; LC method A.

### Example 483: Preparation of Compound 1522

### Step 1: 3-(Difluoromethoxy)-N-[4-(2-fluorophenoxy)-6-(2-isopropylphenyl)pyrimidin-2-yl]benzenesulfonamide

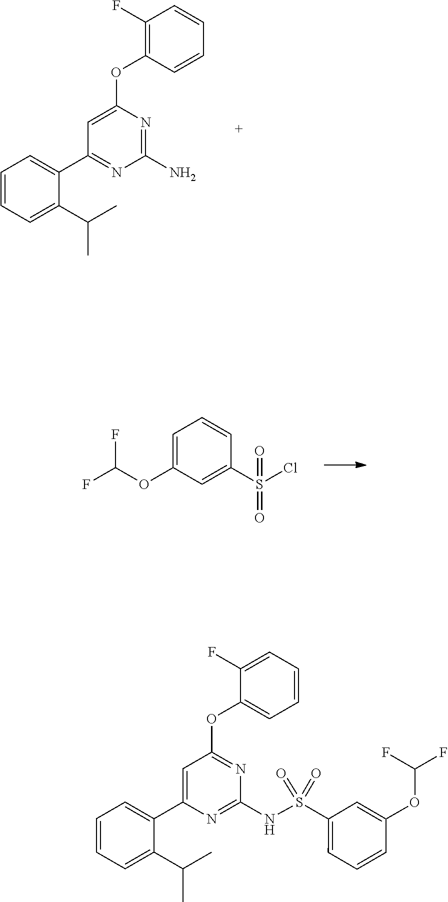 [SMILES: CC(C)c1ccccc1-c1cc(Oc2ccccc2F)nc(N)n1 ; O=S(=O)(Cl)c1cccc(OC(F)F)c1 ; CC(C)c1ccccc1-c1cc(Oc2ccccc2F)nc(NS(=O)(=O)c2cccc(OC(F)F)c2)n1]

In a 3-mL vial, 4-(2-fluorophenoxy)-6-(2-isopropylphenyl)pyrimidin-2-amine (25.00 mg, 0.0773 mmol) (25.0 mg, 0.0773 mmol) and 3-(difluoromethoxy)benzenesulfonyl chloride (36.39 mg, 24.12 μL, 0.15 mmol) were dissolved in pyridine (800 μL). This mixture was heated at 90° C. for 24 hours, after which it was cooled to room temperature and diluted with ethyl acetate (1 mL). This solution was washed with 1 N HCl (3×1 mL), filtered, and purified by reverse phase HPLC (1-99% acetonitrile in water using HCl as a modifier) to give 3-(difluoromethoxy)-N-[4-(2-fluorophenoxy)-6-(2-isopropylphenyl)pyrimidin-2-yl]benzenesulfonamide (7.2 mg, 18%). ¹H NMR (400 MHz, dimethylsulfoxide-d₆) δ 12.42 -11.60 (bs, 1H, D20 exchangeable), 7.51-6.98 (m, 12H), 7.18 (t, J=73.5 Hz, 1H), 6.82 (s, 1H), 3.25-3.00 (m, 1H), 1.13 (d, J=6.8 Hz, 6H). ESI-MS m/z calc. 529.1283, found 530.2 (M+1)⁺; Retention time: 2.11 minutes; LC method A.

### Example 484: Preparation of Compound 1523 Step 1: 3-Chloro-N-[4-(2-fluorophenoxy)-6-(2-isopropylphenyl)pyrimidin-2-yl]benzenesulfonamide

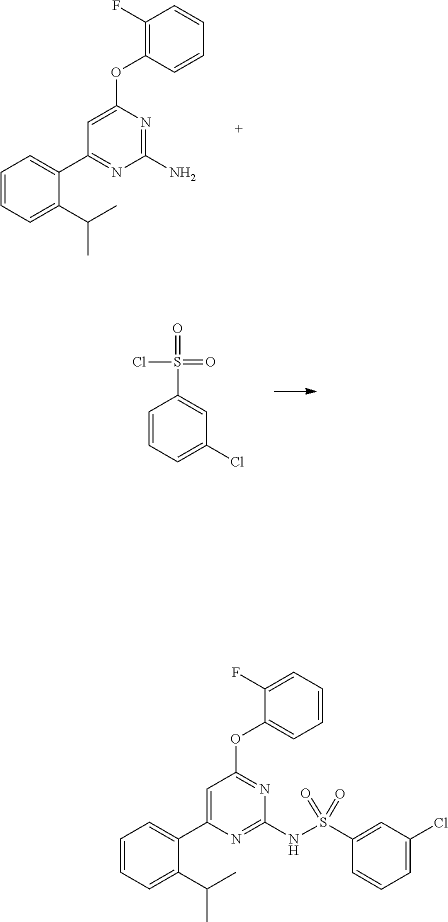 [SMILES: CC(C)c1ccccc1-c1cc(Oc2ccccc2F)nc(N)n1 ; O=S(=O)(Cl)c1cccc(Cl)c1 ; CC(C)c1ccccc1-c1cc(Oc2ccccc2F)nc(NS(=O)(=O)c2cccc(Cl)c2)n1]

In a 3-mL vial, 4-(2-fluorophenoxy)-6-(2-isopropylphenyl)pyrimidin-2-amine (25.00 mg, 0.0773 mmol) (25.0 mg, 0.0773 mmol) and 3-chlorobenzenesulfonyl chloride (31.66 mg, 21.12 μL, 0.15 mmol) were dissolved in pyridine (800 μL). This mixture was heated at 90° C. for 24 hours, after which it was cooled to room temperature and diluted with ethyl acetate (1 mL). This solution was washed with 1 N HCl (3×1 mL), filtered, and purified by reverse phase HPLC (1-99% acetonitrile in water using HCl as a modifier) to give 3-chloro-N-[4-(2-fluorophenoxy)-6-(2-isopropylphenyl)pyrimidin-2-yl]benzenesulfonamide (14.7 mg, 38%). ESI-MS m/z calc. 497.09763, found 498.1 (M+1)⁺; Retention time: 2.17 minutes; LC method A.

### Example 485: Preparation of Compound 1524

### Step 1: N-[4-(2-fluorophenoxy)-6-(2-isopropylphenyl)pyrimidin-2-yl]-3-(trifluoromethyl)benzenesulfonamide

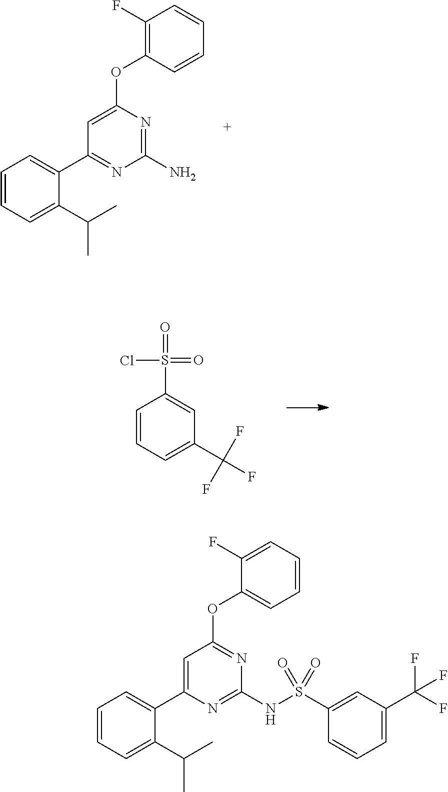 [SMILES: CC(C)c1ccccc1-c1cc(Oc2ccccc2F)nc(N)n1 ; O=S(=O)(Cl)c1cccc(C(F)(F)F)c1 ; CC(C)c1ccccc1-c1cc(Oc2ccccc2F)nc(NS(=O)(=O)c2cccc(C(F)(F)F)c2)n1]

In a 3-mL vial, 4-(2-fluorophenoxy)-6-(2-isopropylphenyl)pyrimidin-2-amine (25.00 mg, 0.0773 mmol) (25.0 mg, 0.0773 mmol) and 3-(trifluoromethyl)benzenesulfonyl chloride (36.69 mg, 0.15 mmol) were dissolved in pyridine (800 μL). This mixture was heated at 90° C. for 24 hours, after which it was cooled to room temperature and diluted with ethyl acetate (1 mL). This solution was washed with 1 N HCl (3×1 mL), filtered, and purified by reverse phase HPLC (1-99% acetonitrile in water using HCl as a modifier) to give N-[4-(2-fluorophenoxy)-6-(2-isopropylphenyl)pyrimidin-2-yl]-3-(trifluoromethyl)benzenesulfonamide (15.0 mg, 37%). ESI-MS m/z calc. 531.12396, found 532.2 (M+1)⁺; Retention time: 2.2 minutes; LC method A.

### Example 486: Preparation of Compound 1525

### Step 1: N-[4-(2-fluorophenoxy)-6-(2-isopropylphenyl)pyrimidin-2-yl]-3-methyl-benzenesulfonamide

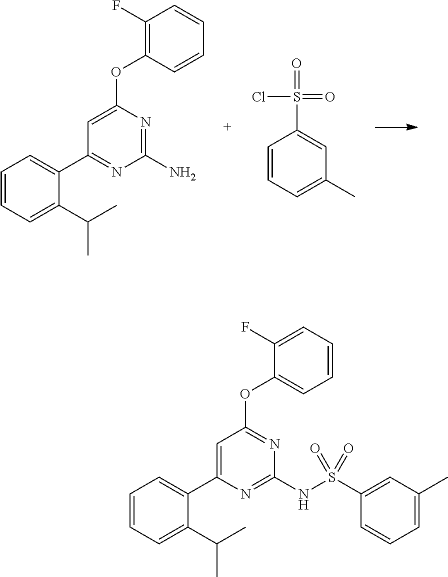 [SMILES: CC(C)c1ccccc1-c1cc(Oc2ccccc2F)nc(N)n1 ; Cc1cccc(S(=O)(=O)Cl)c1 ; Cc1cccc(S(=O)(=O)Nc2nc(Oc3ccccc3F)cc(-c3ccccc3C(C)C)n2)c1]

In a 3-mL vial, 4-(2-fluorophenoxy)-6-(2-isopropylphenyl)pyrimidin-2-amine (25.00 mg, 0.0773 mmol) (25.0 mg, 0.0773 mmol) and 3-methylbenzenesulfonyl chloride (28.60 mg, 21.77 μL, 0.15 mmol) were dissolved in pyridine (800 μL). This mixture was heated at 90° C. for 24 hours, after which it was cooled to room temperature and diluted with ethyl acetate (1 mL). This solution was washed with 1 N HCl (3×1 mL), filtered, and purified by reverse phase HPLC (1-99% acetonitrile in water using HCl as a modifier) to give N-[4-(2-fluorophenoxy)-6-(2-isopropylphenyl)pyrimidin-2-yl]-3-methyl-benzenesulfonamide (13.4 mg, 36%). ESI-MS m z calc. 477.15225, found 478.2 (M+1)⁺; Retention time: 2.16 minutes; LC method A.

### Example 487: Preparation of Compound 1526 and Compound 1527

### Step 1: N-[4-(2-fluorophenoxy)-6-(2-isopropylphenyl)pyrimidin-2-yl]-2-methoxy-benzenesulfonamide

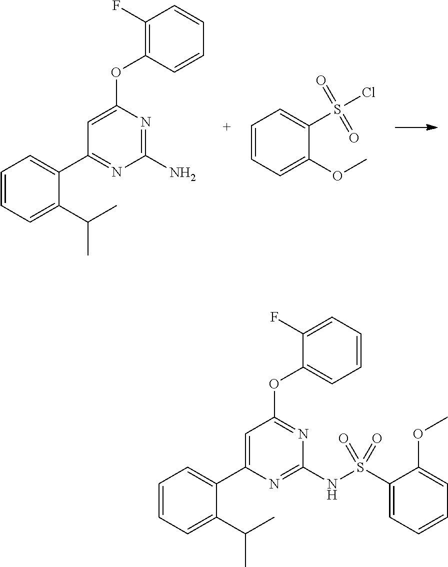 [SMILES: CC(C)c1ccccc1-c1cc(Oc2ccccc2F)nc(N)n1 ; COc1ccccc1S(=O)(=O)Cl ; COc1ccccc1S(=O)(=O)Nc1nc(Oc2ccccc2F)cc(-c2ccccc2C(C)C)n1]

In a 3-mL vial, 4-(2-fluorophenoxy)-6-(2-isopropylphenyl)pyrimidin-2-amine (25.00 mg, 0.0773 mmol) (25.0 mg, 0.0773 mmol) and 2-methoxybenzenesulfonyl chloride (31.00 mg, 0.15 mmol) were dissolved in pyridine (800 μL). This mixture was heated at 90° C. for 24 hours, after which it was cooled to room temperature and diluted with ethyl acetate (1 mL). This solution was washed with 1 N HCl (3×1 mL), filtered, and purified by reverse phase HPLC (1-99% acetonitrile in water using HCl as a modifier) to give N-[4-(2-fluorophenoxy)-6-(2-isopropylphenyl)pyrimidin-2-yl]-2-methoxy-benzenesulfonamide (11.6 mg, 30%). ESI-MS m z calc. 493.14716, found 494.2 (M+1)⁺; Retention time: 2.06 minutes; LC method A.

### Step 2: N-[4-(2-fluorophenoxy)-6-(2-isopropylphenyl)pyrimidin-2-yl]-2-hydroxy-benzenesulfonamide

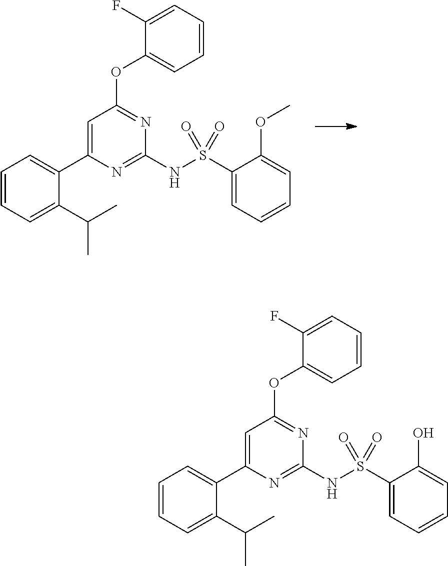 [SMILES: COc1ccccc1S(=O)(=O)Nc1nc(Oc2ccccc2F)cc(-c2ccccc2C(C)C)n1 ; CC(C)c1ccccc1-c1cc(Oc2ccccc2F)nc(NS(=O)(=O)c2ccccc2O)n1]

In a 3 mL vial with a pressure-relief cap, N-[4-(2-fluorophenoxy)-6-(2-isopropylphenyl)pyrimidin-2-yl]-2-methoxy-benzenesulfonamide (6.7 mg, 0.01358 mmol), DCM (400 μL) and a solution of BBr₃ (100 μL of 1.0 M, 0.1000 mmol) in DCM were added. This solution was heated to 50° C. for 15 minutes, after which it was cooled to room temperature, quenched with methanol (500 μL), filtered, and purified by reverse phase HPLC (1-99% acetonitrile in water using HCl as a modifier) to give N-[4-(2-fluorophenoxy)-6-(2-isopropylphenyl)pyrimidin-2-yl]-2-hydroxy-benzenesulfonamide (2.2 mg, 34%). ESI-MS m z calc. 479.1315, found 480.2 (M+1)⁺; Retention time: 2.0 minutes; LC method A.

### Example 488: Preparation of Compound 1528

### Step 1: 2-Amino-N-[4-(2-fluorophenoxy)-6-(2-isopropylphenyl)pyrimidin-2-yl]benzenesulfonamide

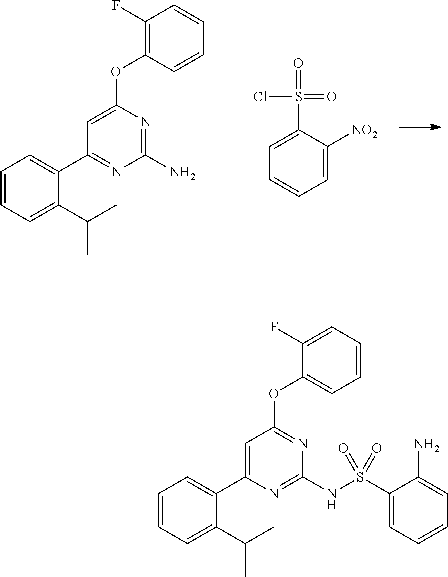 [SMILES: CC(C)c1ccccc1-c1cc(Oc2ccccc2F)nc(N)n1 ; O=[N+]([O-])c1ccccc1S(=O)(=O)Cl ; CC(C)c1ccccc1-c1cc(Oc2ccccc2F)nc(NS(=O)(=O)c2ccccc2N)n1]

In a 3-mL vial, 4-(2-fluorophenoxy)-6-(2-isopropylphenyl)pyrimidin-2-amine (25.00 mg, 0.0773 mmol) (25.0 mg, 0.0773 mmol) and 2-nitrobenzenesulfonyl chloride (33.24 mg, 0.15 mmol) were dissolved in pyridine (800 μL). This mixture was heated at 90° C. for 24 hours, after which it was cooled to room temperature and diluted with ethyl acetate (1 mL). This solution was washed with 1 N HCl (3×1 mL), filtered, and purified by reverse phase HPLC (1-99% acetonitrile in water using HCl as a modifier) to give a pure nitro intermediate. To a 10-mL microwave vial, the purified nitro intermediate was dissolved in 1:1 EtOH:EtOAc (2.0 mL) and this solution was sparged with a balloon of hydrogen gas for 5 minutes. The cap was briefly removed, and 10% Pd(OH)₂/C (5.4 mg, 0.0038 mmol) was added. This reaction mixture was stirred under H₂ (2 L, 80 mmol) at 60° C. for 2 hours. This mixture was cooled to room temperature, filtered through Celite and rinsed with MeOH (5 mL). Concentration in vacuo, re-dissolution in 1:1 MeOH:DMSO (1.0 mL), filtration and purification by reverse phase HPLC (1-99% acetonitrile in water using HCl as a modifier) gave 2-amino-N-[4-(2-fluorophenoxy)-6-(2-isopropylphenyl)pyrimidin-2-yl]benzenesulfonamide (hydrochloride salt) (3.2 mg, 8%). ESI-MS m/z calc. 478.1475, found 479.2 (M+1)⁺; Retention time: 2.02 minutes; LC method A.

### Example 489: Preparation of Compound 1529

### Step 1: 1-tert-Butyl-N-[4-(2-methylphenoxy)-6-(o-tolyl)pyrimidin-2-yl]pyrazole-4-sulfonamide

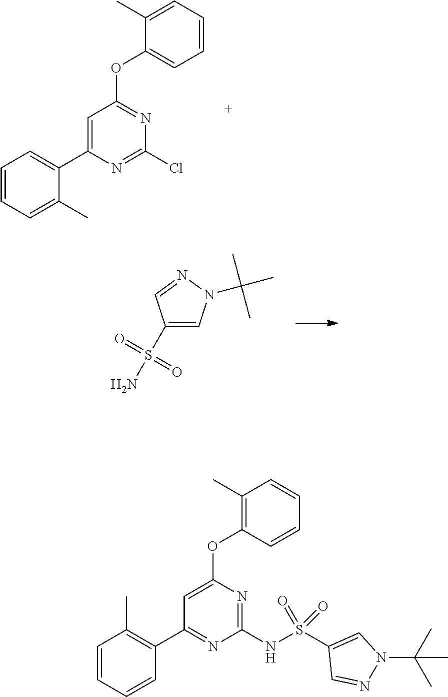 [SMILES: Cc1ccccc1Oc1cc(-c2ccccc2C)nc(Cl)n1 ; CC(C)(C)n1cc(S(N)(=O)=O)cn1 ; Cc1ccccc1Oc1cc(-c2ccccc2C)nc(NS(=O)(=O)c2cnn(C(C)(C)C)c2)n1]

Nitrogen was bubbled through a mixture of 1-tert-butylpyrazole-4-sulfonamide (33.3 mg, 0.1638 mmol), 2-chloro-4-(2-methylphenoxy)-6-(o-tolyl)pyrimidine (43.5 mg, 0.1400 mmol), Xantphos (16.8 mg, 0.02903 mmol), and cesium carbonate (99 mg, 0.3038 mmol) in 1,4-dioxane (1.218 mL) for 5 minutes at room temperature. The reaction was heated at 120° C. for 3.5 hours. The crude product was filtered and purified on reverse phase HPLC (HCl modifier, 30-90% ACN-H₂O) to give 1-tert-butyl-N-[4-(2-methylphenoxy)-6-(o-tolyl)pyrimidin-2-yl]pyrazole-4-sulfonamide (39 mg, 58%). ¹H NMR (400 MHz, DMSO-d₆) δ 11.63 (s, 1H), 8.08 (s, 1H), 7.54-7.27 (m, 8H), 7.23 (s, 1H), 6.66 (s, 1H), 2.38 (s, 3H), 2.14 (s, 3H), 1.33 (s, 10H). ESI-MS m/z calc. 477.18347, found 478.0 (M+1)⁺; Retention time: 2.07 minutes; LC method A.

### Example 490: Preparation of Compound 1530

### Step 1: 3-[[4-[(4-tert-Butylphenyl)methoxy]-6-(2,6-dimethylphenyl)pyrimidin-2-yl]sulfamoyl]benzoic acid

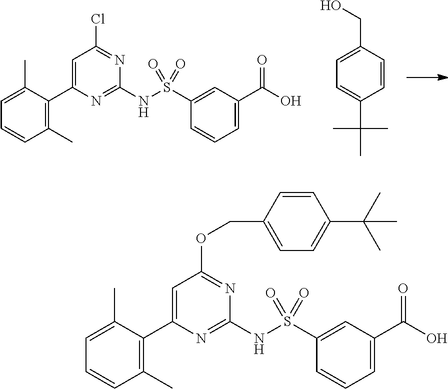

To a solution of 3-[[4-chloro-6-(2,6-dimethylphenyl)pyrimidin-2-yl]sulfamoyl]benzoic acid (240 mg, 0.5743 mmol) and (4-tert-butylphenyl)methanol (72.6 mg, 0.4420 mmol) in THE (1.9 mL) and DMF (260 μL) was added potassium tert-butoxide (273 mg, 2.433 mmol). The reaction was stirred for 1 hour and then concentrated in vacuo. The crude solution was acidified with hydrochloric acid (220 μL of 12 M, 2.640 mmol) and further dissolved in DMSO (3 mL). The sample was purified by reverse phase HPLC (Waters Sunfire C₁₈ column (100×50 mm, 10 m particle size), gradient: 1-99% acetonitrile in water (5 mM HCl) over 15.0 minutes) to afford 3-[[4-[(4-tert-butylphenyl)methoxy]-6-(2,6-dimethylphenyl)pyrimidin-2-yl]sulfamoyl]benzoic acid (66 mg, 27%). ESI-MS m/z calc. 545.1984, found 546.33 (M+1)⁺; Retention time: 0.81 minutes; LC method D.

### Step 2: 3-[[4-[(4-tert-Butylphenyl)methoxy]-6-(2,6-dimethylphenyl)pyrimidin-2-yl]sulfamoyl]-N-ethyl-benzamide

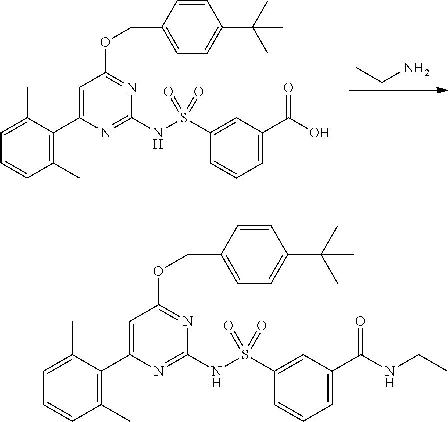

To a solution of 3-[[4-[(4-tert-butylphenyl)methoxy]-6-(2,6-dimethylphenyl)pyrimidin-2-yl]sulfamoyl]benzoic acid (15 mg, 0.02749 mmol) in DMF (150 μL) was added HATU (15.7 mg, 0.04129 mmol). After 5 minutes, ethanamine (40 μL of 2 M in methanol, 0.08000 mmol) and triethylamine (15 μL, 0.1076 mmol) were introduced. The reaction was stirred for 1 hours. The sample was purified by reverse phase HPLC (Phenomenex Luna C₁₈ column (75×30 mm, 5 m particle size), gradient: 1-99% acetonitrile in water (5 mM HCl) over 15.0 minutes) to afford 3-[[4-[(4-tert-butylphenyl)methoxy]-6-(2,6-dimethylphenyl)pyrimidin-2-yl]sulfamoyl]-N-ethyl-benzamide (3.4 mg, 22%). ESI-MS m/z calc. 572.2457, found 573.36 (M+1)⁺; Retention time: 2.09 minutes; LC method A.

### Example 491: Characterization of Compounds 1531-1612

The compounds in the following tables were prepared in a manner analogous to that described above using commercially available reagents and intermediates described herein.

| | | LCMS | | | |
|---|---|---|---|---|---|
| | | Room | | | |
| Compound | | Temperature | Calc. | | LCMS |
| number | Structure | (min) | mass | M + 1 | Method |
| 1531 | 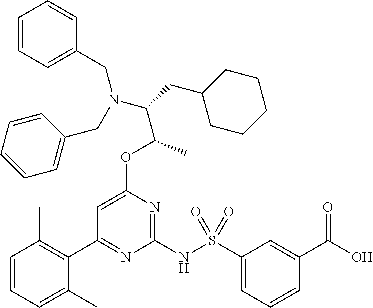 [SMILES: Cc1cccc(C)c1-c1cc(O[C@@H](C)[C@@H](CC2CCCCC2)N(Cc2ccccc2)Cc2ccccc2)nc(NS(=O)(=O)c2cccc(C(=O)O)c2)n1] | 0.86 | 732.335 | 733.5 | D |
| 1532 | 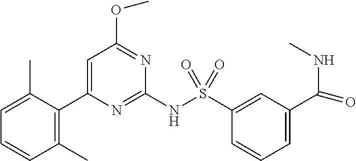 [SMILES: CNC(=O)c1cccc(S(=O)(=O)Nc2nc(OC)cc(-c3c(C)cccc3C)n2)c1] | 1.27 | 426.136 | 427.22 | A |
| 1533 | 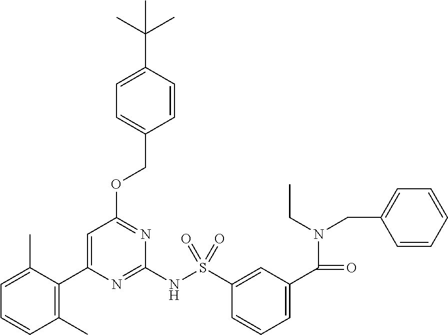 [SMILES: CCN(Cc1ccccc1)C(=O)c1cccc(S(=O)(=O)Nc2nc(OCc3ccc(C(C)(C)C)cc3)cc(-c3c(C)cccc3C)n2)c1] | 2.35 | 662.293 | 663.38 | A |
| 1534 | 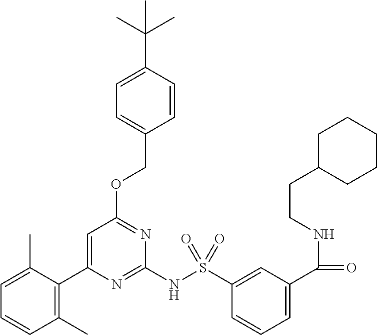 [SMILES: Cc1cccc(C)c1-c1cc(OCc2ccc(C(C)(C)C)cc2)nc(NS(=O)(=O)c2cccc(C(=O)NCCC3CCCCC3)c2)n1] | 2.48 | 654.324 | 655.42 | A |
| 1535 | 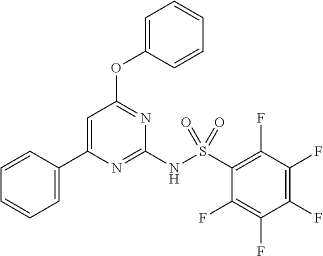 [SMILES: O=S(=O)(Nc1nc(Oc2ccccc2)cc(-c2ccccc2)n1)c1c(F)c(F)c(F)c(F)c1F] | 1.97 | 493.052 | 494.34 | A |
| 1536 | 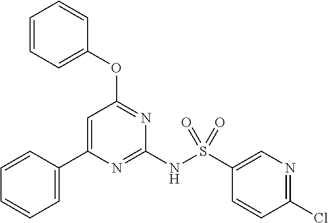 [SMILES: O=S(=O)(Nc1nc(Oc2ccccc2)cc(-c2ccccc2)n1)c1ccc(Cl)nc1] | 1.85 | 438.055 | 439.29 | A |
| 1537 | 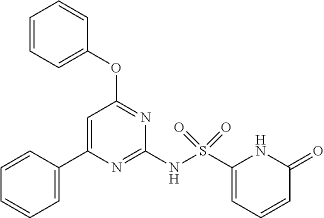 [SMILES: O=c1cccc(S(=O)(=O)Nc2nc(Oc3ccccc3)cc(-c3ccccc3)n2)[nH]1] | 1.495 | 420.089 | 422.37 | A |
| 1538 | 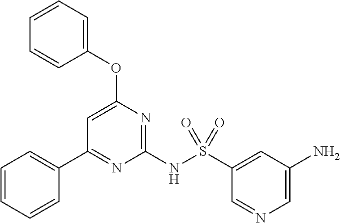 [SMILES: Nc1cncc(S(=O)(=O)Nc2nc(Oc3ccccc3)cc(-c3ccccc3)n2)c1] | 0.98 | 419.105 | 421.32 | A |
| 1540 | 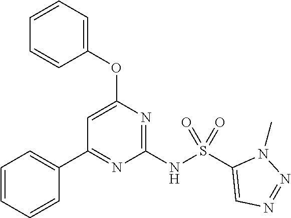 [SMILES: Cn1nncc1S(=O)(=O)Nc1nc(Oc2ccccc2)cc(-c2ccccc2)n1] | 1.4 | 408.1 | 409.32 | A |
| 1541 | 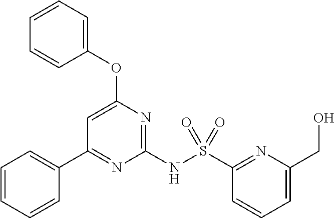 [SMILES: O=S(=O)(Nc1nc(Oc2ccccc2)cc(-c2ccccc2)n1)c1cccc(CO)n1] | 1.26 | 434.105 | 436.34 | A |
| 1542 | 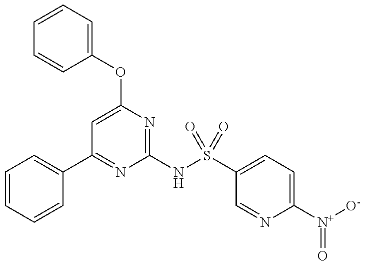 [SMILES: O=[N+]([O-])c1ccc(S(=O)(=O)Nc2nc(Oc3ccccc3)cc(-c3ccccc3)n2)cn1] | 1.4 | 449.079 | 451.32 | A |
| 1543 | 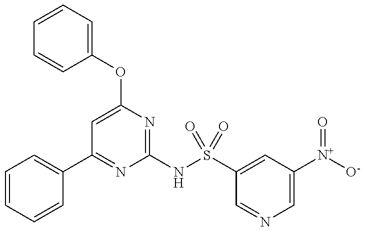 [SMILES: O=[N+]([O-])c1cncc(S(=O)(=O)Nc2nc(Oc3ccccc3)cc(-c3ccccc3)n2)c1] | 1.42 | 449.079 | 451.32 | A |
| 1544 | 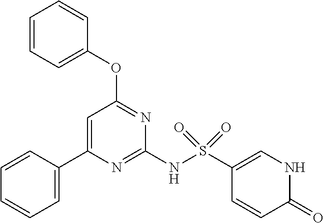 [SMILES: O=c1ccc(S(=O)(=O)Nc2nc(Oc3ccccc3)cc(-c3ccccc3)n2)c[nH]1] | 1.17 | 420.089 | 422.3 | A |
| 1545 | 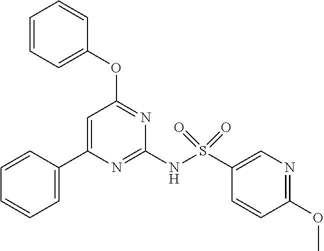 [SMILES: COc1ccc(S(=O)(=O)Nc2nc(Oc3ccccc3)cc(-c3ccccc3)n2)cn1] | 1.87 | 434.105 | 436.37 | A |
| 1546 | 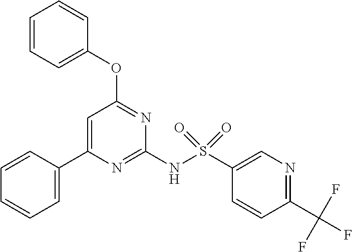 [SMILES: O=S(=O)(Nc1nc(Oc2ccccc2)cc(-c2ccccc2)n1)c1ccc(C(F)(F)F)nc1] | 1.9 | 472.082 | 474.36 | A |
| 1547 | 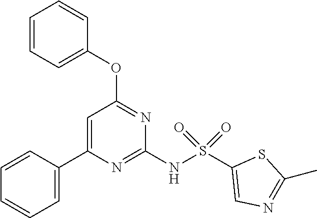 [SMILES: Cc1ncc(S(=O)(=O)Nc2nc(Oc3ccccc3)cc(-c3ccccc3)n2)s1] | 1.64 | 424.066 | 426.1 | A |
| 1548 | 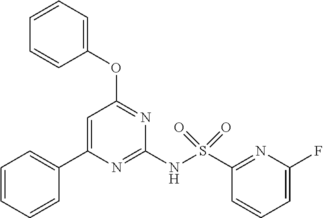 [SMILES: O=S(=O)(Nc1nc(Oc2ccccc2)cc(-c2ccccc2)n1)c1cccc(F)n1] | 1.42 | 422.085 | 424.36 | A |
| 1549 | 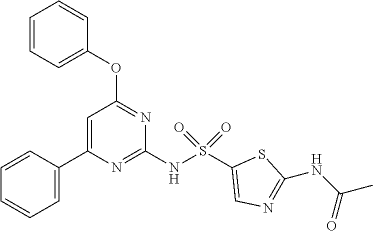 [SMILES: CC(=O)Nc1ncc(S(=O)(=O)Nc2nc(Oc3ccccc3)cc(-c3ccccc3)n2)s1] | 1.58 | 467.072 | 469.39 | A |
| 1550 | 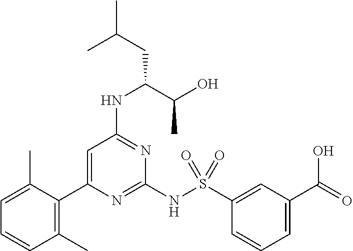 [SMILES: Cc1cccc(C)c1-c1cc(N[C@H](CC(C)C)[C@H](C)O)nc(NS(=O)(=O)c2cccc(C(=O)O)c2)n1] | 1.27 | 512.209 | 513.5 | A |
| 1551 | 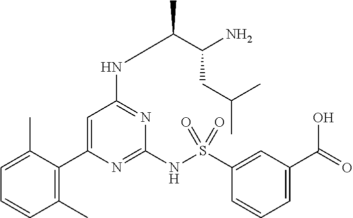 [SMILES: Cc1cccc(C)c1-c1cc(N[C@@H](C)[C@H](N)CC(C)C)nc(NS(=O)(=O)c2cccc(C(=O)O)c2)n1] | 1.11 | 512.209 | 513.5 | A |
| 1552 | 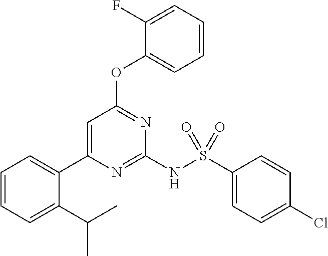 [SMILES: CC(C)c1ccccc1-c1cc(Oc2ccccc2F)nc(NS(=O)(=O)c2ccc(Cl)cc2)n1] | 2.2 | 497.098 | 498.1 | A |
| 1553 | 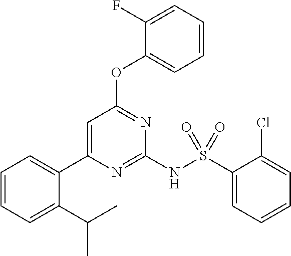 [SMILES: CC(C)c1ccccc1-c1cc(Oc2ccccc2F)nc(NS(=O)(=O)c2ccccc2Cl)n1] | 2.09 | 497.098 | 498.1 | A |
| 1554 | 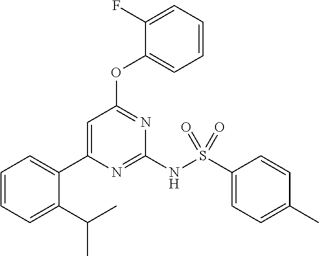 [SMILES: Cc1ccc(S(=O)(=O)Nc2nc(Oc3ccccc3F)cc(-c3ccccc3C(C)C)n2)cc1] | 2.15 | 477.152 | 478.2 | A |
| 1555 | 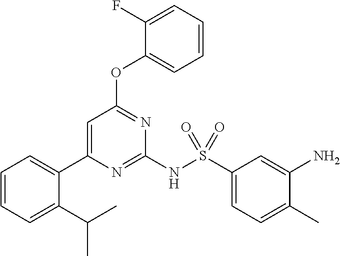 [SMILES: Cc1ccc(S(=O)(=O)Nc2nc(Oc3ccccc3F)cc(-c3ccccc3C(C)C)n2)cc1N] | 1.9 | 492.163 | 493.3 | A |
| 1556 | 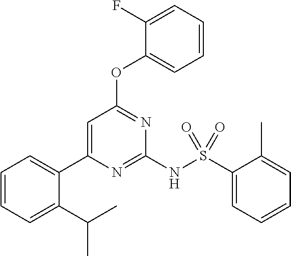 [SMILES: Cc1ccccc1S(=O)(=O)Nc1nc(Oc2ccccc2F)cc(-c2ccccc2C(C)C)n1] | 2.14 | 477.152 | 478.2 | A |
| 1557 | 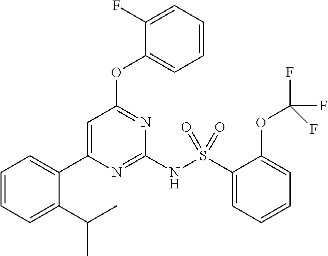 [SMILES: CC(C)c1ccccc1-c1cc(Oc2ccccc2F)nc(NS(=O)(=O)c2ccccc2OC(F)(F)F)n1] | 2.15 | 547.119 | 548.2 | A |
| 1558 | 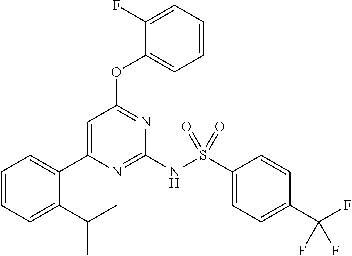 [SMILES: CC(C)c1ccccc1-c1cc(Oc2ccccc2F)nc(NS(=O)(=O)c2ccc(C(F)(F)F)cc2)n1] | 2.21 | 531.124 | 532.2 | A |
| 1559 | 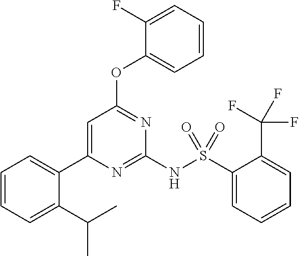 [SMILES: CC(C)c1ccccc1-c1cc(Oc2ccccc2F)nc(NS(=O)(=O)c2ccccc2C(F)(F)F)n1] | 2.11 | 531.124 | 532.2 | A |
| 1560 | 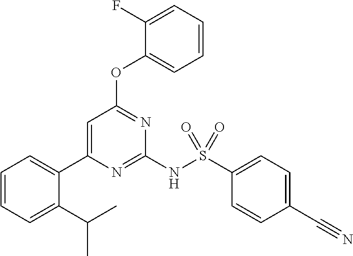 [SMILES: CC(C)c1ccccc1-c1cc(Oc2ccccc2F)nc(NS(=O)(=O)c2ccc(C#N)cc2)n1] | 1.96 | 488.132 | 489.2 | A |
| 1561 | 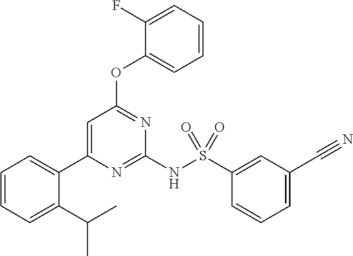 [SMILES: CC(C)c1ccccc1-c1cc(Oc2ccccc2F)nc(NS(=O)(=O)c2cccc(C#N)c2)n1] | 1.96 | 488.132 | 489.2 | A |
| 1562 | 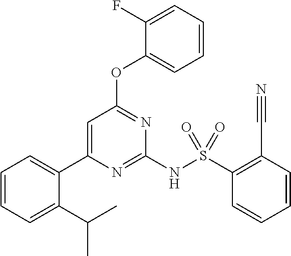 [SMILES: CC(C)c1ccccc1-c1cc(Oc2ccccc2F)nc(NS(=O)(=O)c2ccccc2C#N)n1] | 2 | 488.132 | 489.2 | A |
| 1520 | 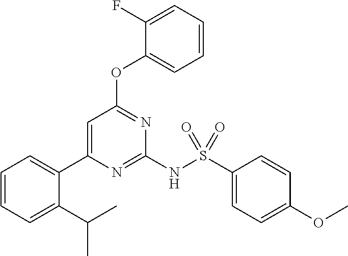 [SMILES: COc1ccc(S(=O)(=O)Nc2nc(Oc3ccccc3F)cc(-c3ccccc3C(C)C)n2)cc1] | 2.09 | 493.147 | 494.2 | A |
| 1526 | 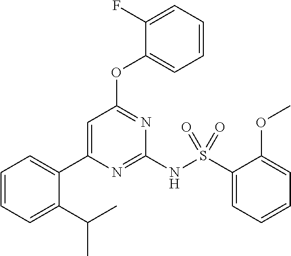 [SMILES: COc1ccccc1S(=O)(=O)Nc1nc(Oc2ccccc2F)cc(-c2ccccc2C(C)C)n1] | 2.06 | 493.147 | 494.2 | A |
| 1565 | 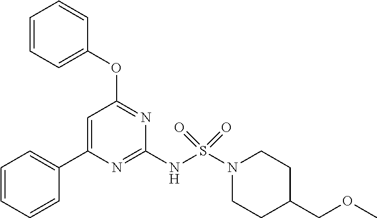 [SMILES: COCC1CCN(S(=O)(=O)Nc2nc(Oc3ccccc3)cc(-c3ccccc3)n2)CC1] | 1.97 | 454.167 | 455.51 | A |
| 1566 | 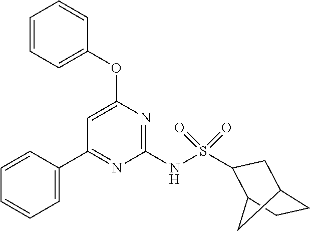 [SMILES: O=S(=O)(Nc1nc(Oc2ccccc2)cc(-c2ccccc2)n1)C1CC2CCC1C2] | 2.09 | 421.146 | 422.49 | A |
| 1567 | 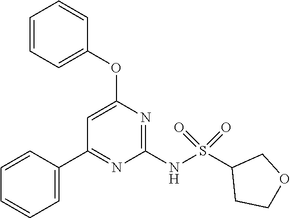 [SMILES: O=S(=O)(Nc1nc(Oc2ccccc2)cc(-c2ccccc2)n1)C1CCOC1] | 1.66 | 397.11 | 398.43 | A |
| 1568 | 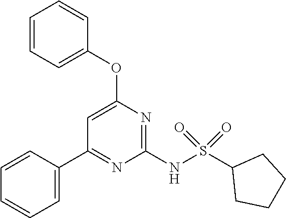 [SMILES: O=S(=O)(Nc1nc(Oc2ccccc2)cc(-c2ccccc2)n1)C1CCCC1] | 1.95 | 395.13 | 396.43 | A |
| 1569 | 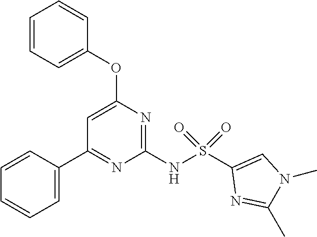 [SMILES: Cc1nc(S(=O)(=O)Nc2nc(Oc3ccccc3)cc(-c3ccccc3)n2)cn1C] | 1.4 | 421.121 | 422.45 | A |
| 1570 | 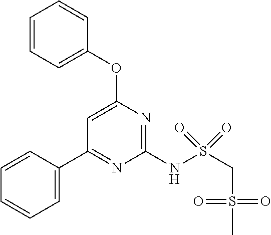 [SMILES: CS(=O)(=O)CS(=O)(=O)Nc1nc(Oc2ccccc2)cc(-c2ccccc2)n1] | 1.43 | 419.061 | 420.39 | A |
| 1571 | 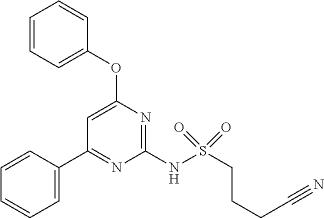 [SMILES: N#CCCCS(=O)(=O)Nc1nc(Oc2ccccc2)cc(-c2ccccc2)n1] | 1.69 | 394.11 | 395.42 | D |
| 1572 | 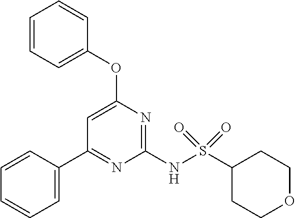 [SMILES: O=S(=O)(Nc1nc(Oc2ccccc2)cc(-c2ccccc2)n1)C1CCOCC1] | 1.71 | 411.125 | 412.47 | A |
| 1573 | 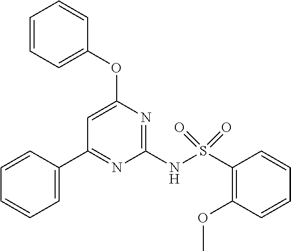 [SMILES: COc1ccccc1S(=O)(=O)Nc1nc(Oc2ccccc2)cc(-c2ccccc2)n1] | 1.88 | 433.11 | 434.46 | A |
| 1574 | 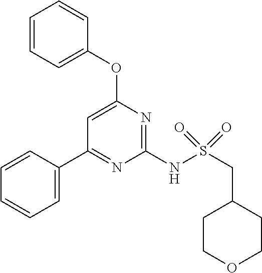 [SMILES: O=S(=O)(CC1CCOCC1)Nc1nc(Oc2ccccc2)cc(-c2ccccc2)n1] | 1.75 | 425.141 | 426.48 | A |
| 1575 | 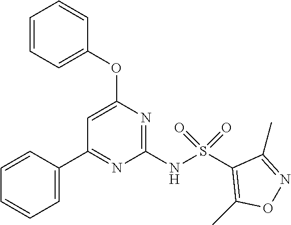 [SMILES: Cc1noc(C)c1S(=O)(=O)Nc1nc(Oc2ccccc2)cc(-c2ccccc2)n1] | 1.86 | 422.105 | 423.43 | A |
| 1576 | 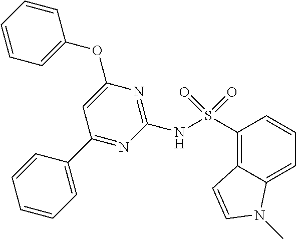 [SMILES: Cn1ccc2c(S(=O)(=O)Nc3nc(Oc4ccccc4)cc(-c4ccccc4)n3)cccc21] | 0.99 | 456.126 | 457.5 | Q |
| 1577 | 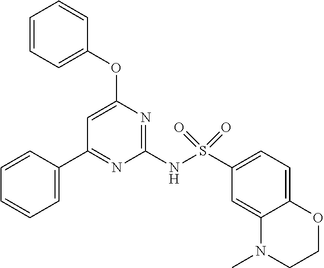 [SMILES: CN1CCOc2ccc(S(=O)(=O)Nc3nc(Oc4ccccc4)cc(-c4ccccc4)n3)cc21] | 1.95 | 474.136 | 475.5 | A |
| 1578 | 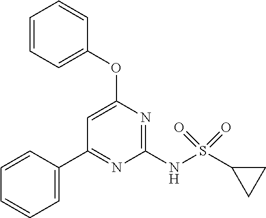 [SMILES: O=S(=O)(Nc1nc(Oc2ccccc2)cc(-c2ccccc2)n1)C1CC1] | 1.78 | 367.099 | 368.42 | A |
| 1579 | 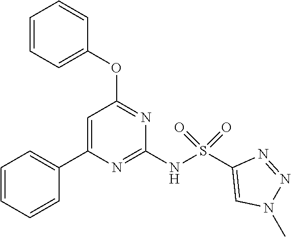 [SMILES: Cn1cc(S(=O)(=O)Nc2nc(Oc3ccccc3)cc(-c3ccccc3)n2)nn1] | 1.5 | 408.1 | 409.2 | A |
| 1580 | 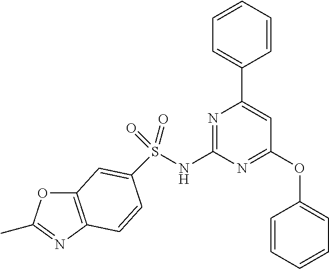 [SMILES: Cc1nc2ccc(S(=O)(=O)Nc3nc(Oc4ccccc4)cc(-c4ccccc4)n3)cc2o1] | 2.55 | 458.105 | 459.4 | I |
| 1581 | 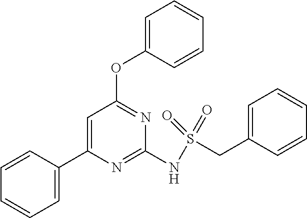 [SMILES: O=S(=O)(Cc1ccccc1)Nc1nc(Oc2ccccc2)cc(-c2ccccc2)n1] | 2.73 | 417.115 | 418.3 | I |
| 1582 | 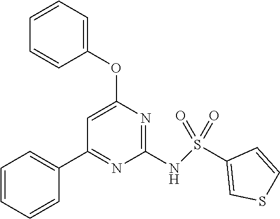 [SMILES: O=S(=O)(Nc1nc(Oc2ccccc2)cc(-c2ccccc2)n1)c1ccsc1] | 2.6 | 409.055 | 410.3 | I |
| 1583 | 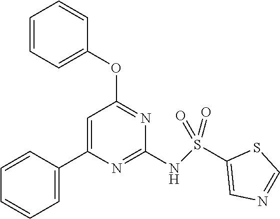 [SMILES: O=S(=O)(Nc1nc(Oc2ccccc2)cc(-c2ccccc2)n1)c1cncs1] | 2.17 | 410.051 | 411.3 | I |
| 1584 | 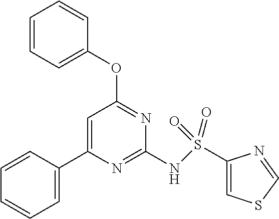 [SMILES: O=S(=O)(Nc1nc(Oc2ccccc2)cc(-c2ccccc2)n1)c1cscn1] | 2.24 | 410.051 | 411.3 | I |
| 1585 | 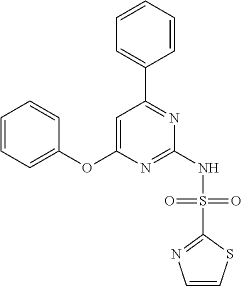 [SMILES: O=S(=O)(Nc1nc(Oc2ccccc2)cc(-c2ccccc2)n1)c1nccs1] | 2.16 | 410.051 | 411.3 | I |
| 1586 | 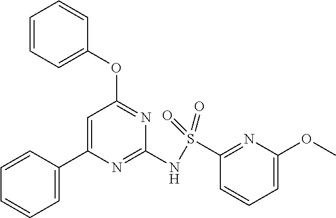 [SMILES: COc1cccc(S(=O)(=O)Nc2nc(Oc3ccccc3)cc(-c3ccccc3)n2)n1] | 2.58 | 434.105 | 435.3 | I |
| 1587 | 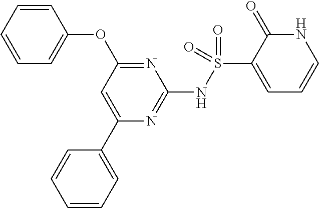 [SMILES: O=c1[nH]cccc1S(=O)(=O)Nc1nc(Oc2ccccc2)cc(-c2ccccc2)n1] | 2.03 | 420.089 | 421.3 | I |
| 1588 | 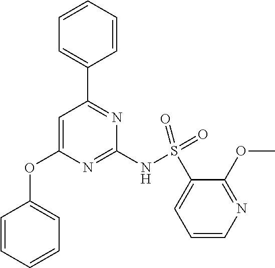 [SMILES: COc1ncccc1S(=O)(=O)Nc1nc(Oc2ccccc2)cc(-c2ccccc2)n1] | 2.55 | 434.105 | 435.4 | I |
| 1589 | 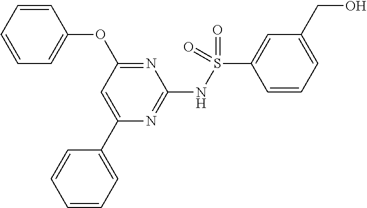 [SMILES: O=S(=O)(Nc1nc(Oc2ccccc2)cc(-c2ccccc2)n1)c1cccc(CO)c1] | 2.31 | 433.11 | 434.4 | I |
| 1590 | 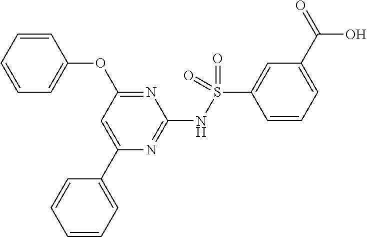 [SMILES: O=C(O)c1cccc(S(=O)(=O)Nc2nc(Oc3ccccc3)cc(-c3ccccc3)n2)c1] | 2.81 | 474.089 | 448.4 | I |
| 1591 | 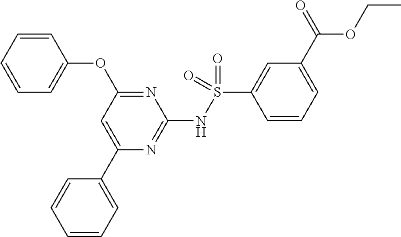 [SMILES: CCOC(=O)c1cccc(S(=O)(=O)Nc2nc(Oc3ccccc3)cc(-c3ccccc3)n2)c1] | 2.83 | 475.12 | 476.4 | I |
| 1592 | 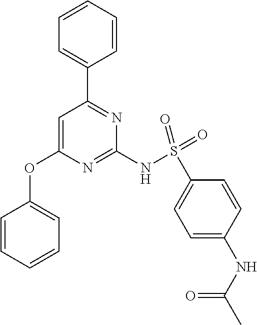 [SMILES: CC(=O)Nc1ccc(S(=O)(=O)Nc2nc(Oc3ccccc3)cc(-c3ccccc3)n2)cc1] | 2.33 | 460.121 | 461.3 | I |
| 1594 | 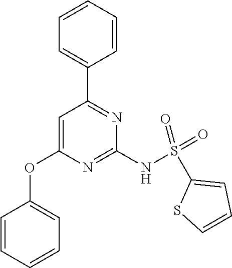 [SMILES: O=S(=O)(Nc1nc(Oc2ccccc2)cc(-c2ccccc2)n1)c1cccs1] | 2.62 | 409.055 | 410.4 | I |
| 1595 | 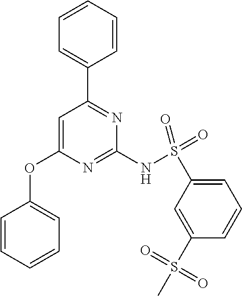 [SMILES: CS(=O)(=O)c1cccc(S(=O)(=O)Nc2nc(Oc3ccccc3)cc(-c3ccccc3)n2)c1] | 2.38 | 481.077 | 482.4 | I |
| 1596 | 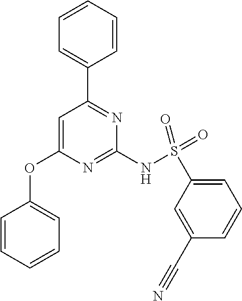 [SMILES: N#Cc1cccc(S(=O)(=O)Nc2nc(Oc3ccccc3)cc(-c3ccccc3)n2)c1] | 2.58 | 428.094 | 429.4 | I |
| 1597 | 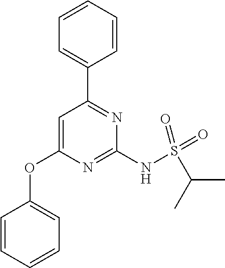 [SMILES: CC(C)S(=O)(=O)Nc1nc(Oc2ccccc2)cc(-c2ccccc2)n1] | 2.54 | 369.115 | 370.4 | I |
| 1598 | 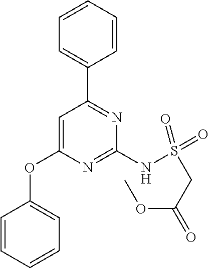 [SMILES: COC(=O)CS(=O)(=O)Nc1nc(Oc2ccccc2)cc(-c2ccccc2)n1] | 2.25 | 399.089 | 400.5 | I |
| 1599 | 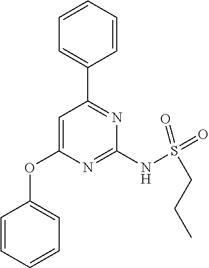 [SMILES: CCCS(=O)(=O)Nc1nc(Oc2ccccc2)cc(-c2ccccc2)n1] | ND | 369.115 | ND | — |
| 1600 | 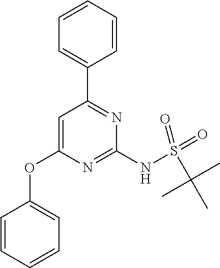 [SMILES: CC(C)(C)S(=O)(=O)Nc1nc(Oc2ccccc2)cc(-c2ccccc2)n1] | 2.65 | 383.13 | 384.5 | I |
| 1601 | 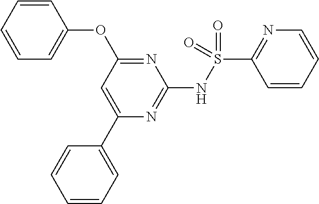 [SMILES: O=S(=O)(Nc1nc(Oc2ccccc2)cc(-c2ccccc2)n1)c1ccccn1] | 2.29 | 404.094 | 405.4 | I |
| 1602 | 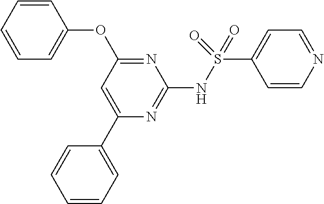 [SMILES: O=S(=O)(Nc1nc(Oc2ccccc2)cc(-c2ccccc2)n1)c1ccncc1] | 1.94 | 404.094 | 405.4 | I |
| 1603 | 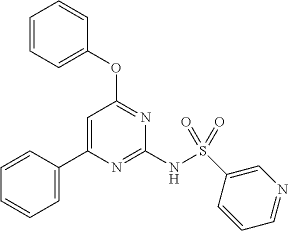 [SMILES: O=S(=O)(Nc1nc(Oc2ccccc2)cc(-c2ccccc2)n1)c1cccnc1] | 2.12 | 404.094 | 405.4 | I |
| 1604 | 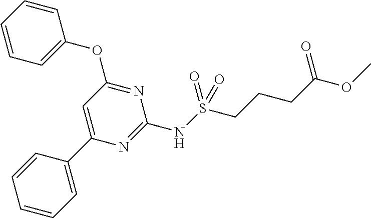 [SMILES: COC(=O)CCCS(=O)(=O)Nc1nc(Oc2ccccc2)cc(-c2ccccc2)n1] | 2.42 | 427.12 | 428.5 | I |
| 1605 | 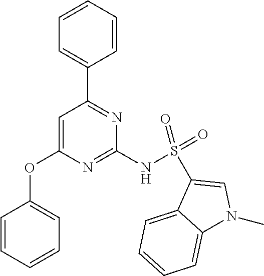 [SMILES: Cn1cc(S(=O)(=O)Nc2nc(Oc3ccccc3)cc(-c3ccccc3)n2)c2ccccc21] | 2.65 | 456.126 | 457.5 | I |
| 1606 | 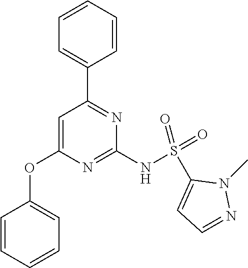 [SMILES: Cn1nccc1S(=O)(=O)Nc1nc(Oc2ccccc2)cc(-c2ccccc2)n1] | 1.65 | 407.105 | 408.4 | I |
| 1607 | 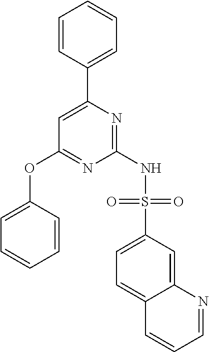 [SMILES: O=S(=O)(Nc1nc(Oc2ccccc2)cc(-c2ccccc2)n1)c1ccc2cccnc2c1] | 1.64 | 454.11 | 455.4 | A |
| 1608 | 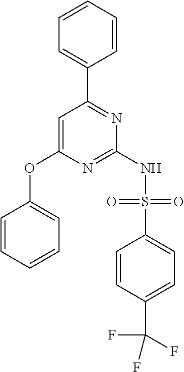 [SMILES: O=S(=O)(Nc1nc(Oc2ccccc2)cc(-c2ccccc2)n1)c1ccc(C(F)(F)F)cc1] | 2.1 | 471.086 | 472.45 | A |
| 1609 | 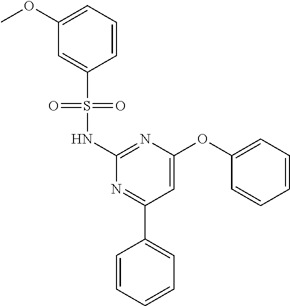 [SMILES: COc1cccc(S(=O)(=O)Nc2nc(Oc3ccccc3)cc(-c3ccccc3)n2)c1] | 1.87 | 433.11 | 434.2 | A |
| 1501 | 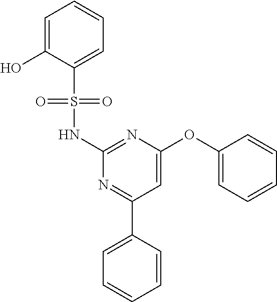 [SMILES: O=S(=O)(Nc1nc(Oc2ccccc2)cc(-c2ccccc2)n1)c1ccccc1O] | 1.82 | 419.094 | 420.4 | A |
| 1611 | 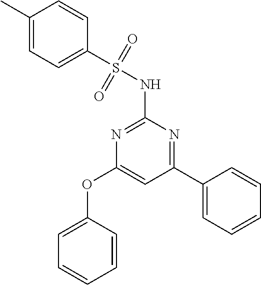 [SMILES: Cc1ccc(S(=O)(=O)Nc2nc(Oc3ccccc3)cc(-c3ccccc3)n2)cc1] | 1.95 | 417.115 | 418.5 | A |
| 1612 | 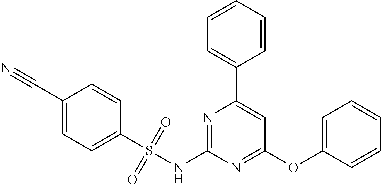 [SMILES: N#Cc1ccc(S(=O)(=O)Nc2nc(Oc3ccccc3)cc(-c3ccccc3)n2)cc1] | 1.83 | 428.094 | 429.5 | A |
| 1596 | 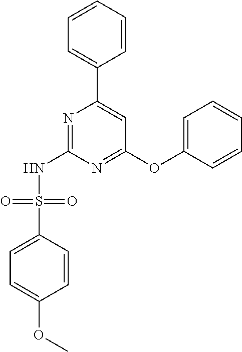 [SMILES: COc1ccc(S(=O)(=O)Nc2nc(Oc3ccccc3)cc(-c3ccccc3)n2)cc1] | 1.92 | 433.11 | 434.5 | A |

| Compound | |
|---|---|
| number | NMR |
| 1568 | ¹H NMR (400 MHz, Chloroform-d) δ 8.04-7.95 (m, 2H), 7.58-7.41 (m, |
| | 5H), 7.35-7.16 (m, 3H), 6.97 (s, 1H), 4.02 (ddd, J = 16.1, 8.8, 7.1 Hz, |
| | 1H), 2.11-1.97 (m, 2H), 1.89-1.61 (m, 6H). |
| 1573 | ¹H NMR (400 MHz, Chloroform-d) δ 7.91-7.84 (m, 2H), 7.70 (d, J = |
| | 7.6 Hz, 1H), 7.51-7.40 (m, 6H), 7.38-7.29 (m, 1H), 7.16-7.08 (m, 2H), |
| | 6.96-6.81 (m, 3H), 3.89 (s, 3H). |
| 1577 | ¹H NMR (400 MHz, Chloroform-d) δ 7.95 (d, J = 6.7 Hz, 2H), 7.71- |
| | 7.39 (m, 6H), 7.23 (d, J = 48.9 Hz, 4H), 7.13-6.97 (m, 1H), 6.84 (q, J = |
| | 4.7 Hz, 1H), 6.68-6.56 (m, 1H), 4.28 (d, J = 7.5 Hz, 2H), 3.22 (d, J = |
| | 7.9 Hz, 2H), 2.75 (q, J = 5.4, 4.8 Hz, 3H). |
| 1580 | ¹H NMR (400 MHz, DMSO) δ 11.85 (s, 1H), 8.11-7.92 (m, 1H), 7.79 |
| | (s, 1H), 7.68 (q, J = 8.4 Hz, 2H), 7.52 (tq, J = 6.2, 2.9 Hz, 5H), 7.36 (t, J = |
| | 7.4 Hz, 1H), 7.27-7.09 (m, 3H), 2.66 (s, 3H). |
| 1581 | ¹H NMR (400 MHz, DMSO) δ 11.22 (s, 1H), 8.19 (dd, J = 6.7, 2.9 Hz, |
| | 2H), 7.61-7.53 (m, 3H), 7.51-7.45 (m, 2H), 7.42 (s, 1H), 7.37-7.28 |
| | (m, 6H), 7.13 (ddd, J = 5.6, 2.9, 1.7 Hz, 2H), 4.55 (s, 2H). |
| 1582 | ¹H NMR (400 MHz, DMSO) δ 11.70 (s, 1H), 8.09-8.03 (m, 2H), 7.75 |
| | (s, 1H), 7.59-7.50 (m, 6H), 7.39-7.33 (m, 1H), 7.30-7.24 (m, 3H), 7.14 |
| | (dd, J = 5.0, 1.4 Hz, 1H). |
| 1583 | ¹H NMR (400 MHz, DMSO) δ 12.37 (s, 1H), 9.26 (s, 1H), 8.12-8.07 |
| | (m, 2H), 7.98 (s, 1H), 7.58-7.48 (m, 5H), 7.37-7.31 (m, 1H), 7.30-7.24 |
| | (m, 3H). |
| 1584 | ¹H NMR (400 MHz, DMSO) δ 12.08 (s, 1H), 9.13 (d, J = 2.0 Hz, 1H), |
| | 8.03-7.87 (m, 3H), 7.55-7.48 (m, 5H), 7.37-7.32 (m, 1H), 7.25-7.20 |
| | (m, 3H). |
| 1585 | ¹H NMR (400 MHz, DMSO) δ 12.72 (s, 1H), 8.04 (d, J = 3.1 Hz, 1H), |
| | 8.01-7.93 (m, 3H), 7.59-7.44 (m, 5H), 7.34-7.28 (m, 1H), 7.23 (s, 1H), |
| | 7.19 (dd, J = 7.4, 1.7 Hz, 2H). |
| 1586 | ¹H NMR (400 MHz, DMSO) δ 11.97 (s, 1H), 8.01-7.90 (m, 2H), 7.80 |
| | (t, J = 7.8 Hz, 1H), 7.54-7.45 (m, 5H), 7.35-7.28 (m, 2H), 7.23 (s, 1H), |
| | 7.19-7.13 (m, 2H), 7.01 (d, J = 8.3 Hz, 1H), 3.78 (s, 3H). |
| 1587 | ¹H NMR (400 MHz, DMSO) δ 12.25 (s, 1H), 11.66 (s, 1H), 8.09-7.95 |
| | (m, 2H), 7.76 (dd, J = 7.2, 2.1 Hz, 1H), 7.65 (s, 1H), 7.56-7.45 (m, 5H), |
| | 7.38-7.31 (m, 1H), 7.25-7.15 (m, 3H), 6.17 (t, J = 6.8 Hz, 1H). |
| 1588 | ¹H NMR (400 MHz, DMSO) δ 11.96 (s, 1H), 8.32 (dd, J = 4.9, 1.9 Hz, |
| | 1H), 8.08-7.92 (m, 2H), 7.78 (d, J = 7.3 Hz, 1H), 7.60-7.47 (m, 5H), |
| | 7.42-7.31 (m, 1H), 7.24-7.13 (m, 3H), 6.97 (dd, J = 7.5, 4.8 Hz, 1H), |
| | 3.91 (s, 3H). |
| 1589 | ¹H NMR (400 MHz, DMSO) δ 11.74 (s, 1H), 8.01 (dd, J = 7.6, 2.2 Hz, |
| | 2H), 7.87 (s, 1H), 7.51 (td, J = 8.2, 7.7, 3.9 Hz, 7H), 7.36 (dt, J = 14.8, |
| | 7.4 Hz, 2H), 7.26-7.19 (m, 3H), 5.41 (t, J = 5.5 Hz, 1H), 4.50 (d, J = |
| | 5.1 Hz, 2H). |
| 1590 | ¹H NMR (400 MHz, DMSO) δ 13.50 (s, 1H), 11.93 (s, 1H), 8.46 (t, J = |
| | 1.8 Hz, 1H), 8.12 (dt, J = 7.7, 1.4 Hz, 1H), 8.03-7.94 (m, 2H), 7.80 (d, |
| | J = 7.8 Hz, 1H), 7.62-7.45 (m, 6H), 7.37-7.30 (m, 1H), 7.27-7.20 (m, |
| | 3H). |
| 1591 | ¹H NMR (400 MHz, DMSO) δ 11.97 (s, 1H), 8.47-8.40 (m, 1H), 8.13 |
| | (dt, J = 7.9, 1.3 Hz, 1H), 8.02-7.95 (m, 2H), 7.82 (d, J = 7.8 Hz, 1H), |
| | 7.62-7.46 (m, 6H), 7.36-7.30 (m, 1H), 7.26-7.20 (m, 3H), 4.34 (q, J = |
| | 7.1 Hz, 2H), 1.30 (t, J = 7.1 Hz, 3H). |
| 1596 | ¹H NMR (400 MHz, DMSO) δ 12.14 (s, 1H), 8.10 (s, 1H), 8.06 (dt, J = |
| | 7.8, 1.4 Hz, 1H), 8.02-7.98 (m, 2H), 7.89 (d, J = 7.7 Hz, 1H), 7.66 (t, J = |
| | 7.9 Hz, 1H), 7.59-7.48 (m, 5H), 7.38-7.32 (m, 1H), 7.28-7.21 (m, |
| | 3H). |
| 1598 | ¹H NMR (400 MHz, DMSO) δ 11.78 (s, 1H), 8.18-81.0 (m, 2H), 7.61- |
| | 7.52 (m, 3H), 7.51-7.42 (m, 2H), 7.38-7.26 (m, 4H), 4.49 (s, 2H), 3.64 |
| | (s, 3H). |
| 1599 | ¹H NMR (400 MHz, DMSO) δ 11.21 (s, 1H), 8.17-8.11 (m, 2H), 7.60- |
| | 7.52 (m, 3H), 7.51-7.44 (m, 2H), 7.35 (s, 1H), 7.33-7.25 (m, 3H), 3.27- |
| | 3.13 (m, 2H), 1.64-1.49 (m, 2H), 0.86 (t, J = 7.4 Hz, 3H). |
| 1601 | ¹H NMR (400 MHz, DMSO) δ 12.04 (s, 1H), 8.67-8.60 (m, 1H), 7.95- |
| | 7.86 (m, 3H), 7.69 (d, J = 7.8 Hz, 1H), 7.58 (ddd, J = 7.7, 4.6, 1.1 Hz, |
| | 1H), 7.54-7.44 (m, 5H), 7.39-7.30 (m, 1H), 7.21 (s, 1H), 7.18-7.08 |
| | (m, 2H). |
| 1602 | ¹H NMR (400 MHz, DMSO) δ 12.23 (s, 1H), 8.72-8.60 (m, 2H), 8.04- |
| | 7.94 (m, 2H), 7.60-7.49 (m, 5H), 7.46 (d, J = 4.9 Hz, 1H), 7.42-7.35 |
| | (m, 1H), 7.27 (s, 1H), 7.25-7.17 (m, 2H). |
| 1603 | ¹H NMR (400 MHz, DMSO) δ 12.10 (bs, 1H), 8.84 (d, J = 2.5 Hz, 1H), |
| | 8.74 (dd, J = 4.9, 1.6 Hz, 1H), 8.05-7.98 (m, 2H), 7.87 (d, J = 7.7 Hz, |
| | 1H), 7.63-7.44 (m, 6H), 7.43-7.30 (m, 1H), 7.30-7.16 (m, 3H). |
| 1604 | ¹H NMR (400 MHz, DMSO) δ 11.28 (s, 1H), 8.24-7.99 (m, 2H), 7.69- |
| | 7.43 (m, 5H), 7.37-7.11 (m, 4H), 3.53 (s, 3H), 3.33 (dd, J = 8.8, 6.5 Hz, |
| | 2H), 2.37 (t, J = 7.3 Hz, 2H), 1.82 (dq, J = 9.8, 7.4 Hz, 2H). |
| 1605 | ¹H NMR (400 MHz, DMSO) δ 11.85 (s, 1H), 7.92-7.87 (m, 1H), 7.85- |
| | 7.78 (m, 3H), 7.57-7.53 (m, 1H), 7.53-7.43 (m, 5H), 7.34-7.19 (m, |
| | 5H), 7.18 (s, 1H), 3.80 (s, 3H). |
| 1607 | ¹H NMR (400 MHz, DMSO) δ 12.01 (s, 0H), 9.07 (dd, J = 4.2, 1.7 Hz, |
| | 1H), 8.58-8.50 (m, 1H), 8.48 (dd, J = 8.3, 1.6 Hz, 1H), 8.09 (d, J = 8.6 |
| | Hz, 1H), 8.00-7.91 (m, 2H), 7.80 (dd, J = 8.4, 1.9 Hz, 1H), 7.70 (dd, J = |
| | 8.3, 4.2 Hz, 1H), 7.58-7.39 (m, 5H), 7.38-7.26 (m, 1H), 7.23-7.16 |
| | (m, 3H). |
| 1608 | ¹H NMR (400 MHz, DMSO) δ 12.15 (s, 0H), 8.06-7.91 (m, 2H), 7.80 |
| | (s, 4H), 7.61-7.49 (m, 5H), 7.44-7.34 (m, 1H), 7.30-7.17 (m, 3H). |
| 1609 | 1H NMR (400 MHz, MeOD) ? 7.92 (t, J = 1.6 Hz, 1H), 7.90 (d, J = 1.9 |
| | Hz, 1H), 7.53-7.41 (m, 6H), 7.35 (t, J = 8.0 Hz, 1H), 7.31-7.25 (m, |
| | 2H), 7.20-7.18 (m, 1H), 7.18-7.12 (m, 3H), 3.80 (s, 3H). |
| 1611 | 1H NMR (400 MHz, MeOD) ? 7.96-7.88 (m, 2H), 7.59-7.43 (m, 7H), |
| | 7.36-7.29 (m, 1H), 7.26-7.15 (m, 4H), 7.07 (s, 1H), 2.37 (s, 3H). |
| 1612 | ¹H NMR (400 MHz, DMSO) δ 12.20 (s, 0H), 8.04-7.96 (m, 8H), 7.90 |
| | (d, J = 7.9 Hz, 8H), 7.74-7.67 (m, 14H), 7.57-7.51 (m, 19H), 7.41- |
| | 7.35 (m, 4H), 7.27 (s, 4H), 7.24-7.19 (m, 8H). |
| 1596 | ¹H NMR (400 MHz, DMSO) δ 11.58 (s, 1H), 8.12-8.03 (m, 2H), 7.59- |
| | 7.51 (m, 7H), 7.40-7.33 (m, 1H), 7.27-7.22 (m, 3H), 6.95-6.90 (m, |
| | 2H), 3.79 (s, 3H). |

### Example 492: Preparation of N-[2-(2,6-Dimethylphenyl)-6-(1-phenylcyclopropyl)pyrimidin-4-yl]-1-methyl-pyrazole-4-sulfonamide

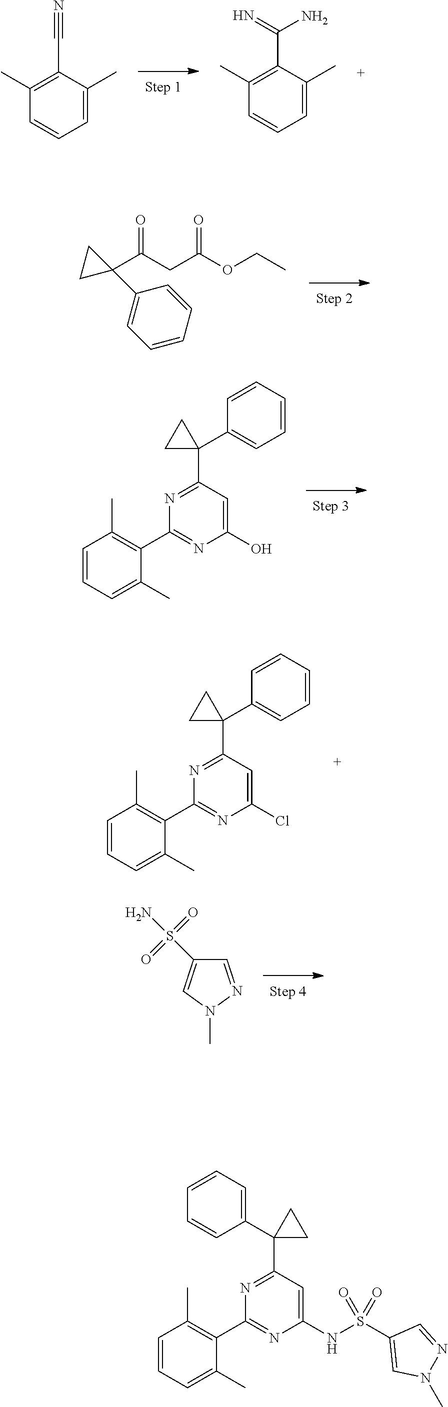

### Step 1: 2,6-Dimethylbenzamidine

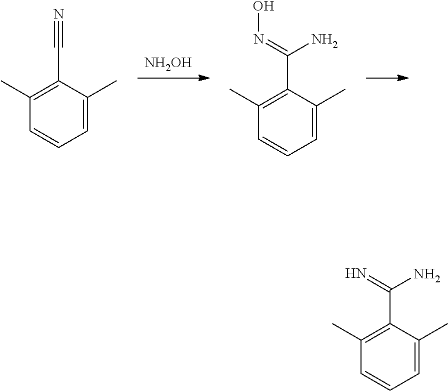

To a solution of 2,6-dimethylbenzonitrile (6 g, 45.74 mmol) and hydroxylamine (hydrochloride salt) (8.3 g, 119.4 mmol) in ethanol (23 mL) was added potassium tert-butoxide (13.3 g, 118.5 mmol). The reaction was sealed in a pressure vessel and heated to 110° C. for 16 hours. The reaction mixture was concentrated in vacuo. The crude residue was partitioned between water and ethyl acetate. The organic layer was separated, and the aqueous layer was extracted once more with ethyl acetate. The combined organics were dried over magnesium sulfate, filtered, and concentrated in vacuo. The crude residue was subjected to flash column chromatography on silica gel (gradient: 10 to 100% ethyl acetate in hexanes) to afford N′-hydroxy-2,6-dimethyl-benzamidine (3.91 g, 16%) ESI-MS m/z calc. 164.09496, found 165.06 (M+1)⁺; Retention time: 0.23 minutes (LC method D).

To a solution of N′-hydroxy-2,6-dimethyl-benzamidine (3.91 g, 16%) in acetic acid (90 mL) was added acetic anhydride (6.5 mL, 68.89 mmol). The reaction was allowed to stir for 15 minutes before adding palladium on carbon (1000 mg of 10% w/w, 0.9397 mmol)(10% by weight). The head space was purged with hydrogen and to the reaction flask was set with a balloon filled with hydrogen gas. The reaction was stirred for 16 hours. The solution was filtered and concentrated in vacuo. The residual acetic acid was removed after azeotroping (3×) with benzene. Without any further purification, this afforded 2,6-dimethylbenzamidine (4.3 g, 20%) as a yellow solid. ESI-MS m/z calc. 148.10005, found 149.03 (M+1)⁺; Retention time: 0.22 minutes (LC method D).

### Step 2: 2-(2,6-Dimethylphenyl)-6-(1-phenylcyclopropyl)pyrimidin-4-ol

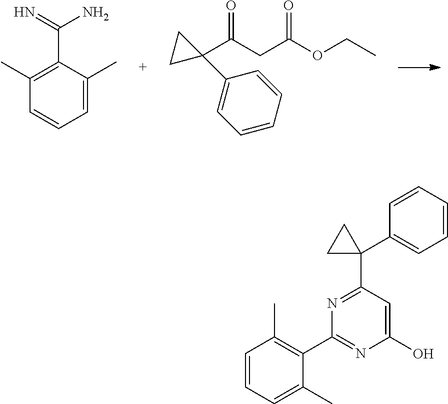

A solution of 2,6-dimethylbenzamidine (1.58 g, 3.305 mmol), ethyl 3-oxo-3-(1-phenylcyclopropyl)propanoate (1 g, 2.755 mmol), and potassium tert-butoxide (1.36 g, 12.12 mmol) in ethanol (9.2 mL) was heated in a sealed vessel to 120° C. for 16 hours. The solution was concentrated, acidified with acetic acid (2.5 mL, 43.96 mmol), and partitioned between ethyl acetate and water. The water layer was removed, and the organic layer was washed with brine, dried over magnesium sulfate, filtered, and concentrated in vacuo. The crude residue was subjected to flash column chromatography on silica gel (gradient: 10 to 100% ethyl acetate in hexanes) to afford 2-(2,6-dimethylphenyl)-6-(1-phenylcyclopropyl)pyrimidin-4-ol (120 mg, 12%) as a white solid. ESI-MS m/z calc. 316.15756, found 317.2 (M+1)⁺; Retention time: 0.7 minutes (LC method D).

### Step 3: 4-Chloro-2-(2,6-dimethylphenyl)-6-(1-phenylcyclopropyl)pyrimidine

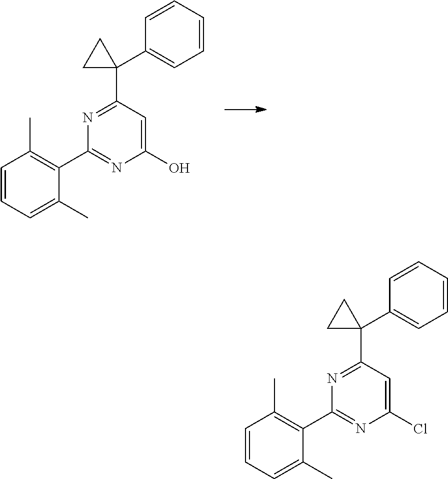 [SMILES: Cc1cccc(C)c1-c1nc(O)cc(C2(c3ccccc3)CC2)n1 ; Cc1cccc(C)c1-c1nc(Cl)cc(C2(c3ccccc3)CC2)n1]

A solution of 2-(2,6-dimethylphenyl)-6-(1-phenylcyclopropyl)pyrimidin-4-ol (120 mg, 0.3793 mmol) in phosphorus oxychloride (425 μL, 4.560 mmol) was heated at 100° C. for 1 hour. The reaction was cooled at poured onto ice. After the ice melted, the reaction flask was immersed in an ice bath and ethyl acetate was added. After 10 minutes of vigorous stirring, the organic layer was separated, and the aqueous layer was extracted ethyl acetate. The combined organics were washed with brine, dried over magnesium sulfate, filtered, and concentrated in vacuo. The crude residue was subjected to flash column chromatography on silica gel (short column, 50% ethyl acetate in hexanes) to afford 4-chloro-2-(2,6-dimethylphenyl)-6-(1-phenylcyclopropyl)pyrimidine (86 mg, 57%) as a yellow solid. ESI-MS m/z calc. 334.1237, found 335.21 (M+1)⁺; Retention time: 0.93 minutes (LC method D).

### Step 4: N-[2-(2,6-Dimethylphenyl)-6-(1-phenylcyclopropyl)pyrimidin-4-yl]-1 -methyl-pyrazole-4-sulfonamide

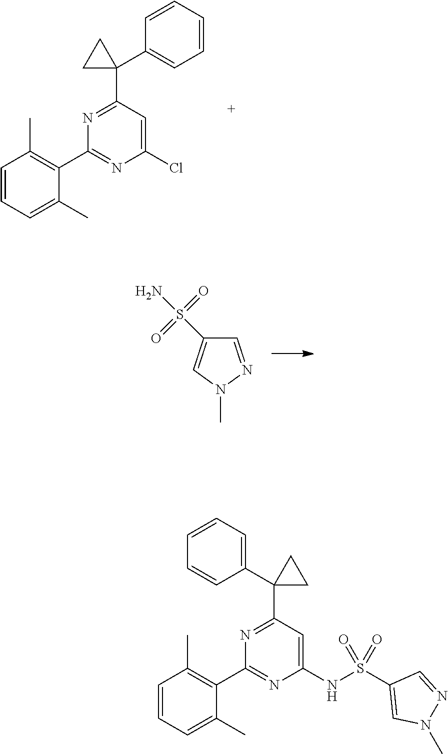

A heterogeneous mixture of 4-chloro-2-(2,6-dimethylphenyl)-6-(1-phenylcyclopropyl)pyrimidine (21.5 mg, 0.05394 mmol), 1-methylpyrazole-4-sulfonamide (26.1 mg, 0.1619 mmol), (5-diphenylphosphanyl-9,9-dimethyl-xanthen-4-yl)-diphenyl-phosphane (12.5 mg, 0.02160 mmol), potassium carbonate (22.4 mg, 0.1621 mmol), and palladium(II) acetate (2.4 mg, 0.01069 mmol) in dioxane (1.1 mL) was microwaved at 125° C. for 25 minutes. The solvent was removed under a steady stream of air and the crude residue was dissolved in DMSO (1.0 mL), acidified with acetic acid (60 μL, 1.055 mmol), and filtered. The sample was purified by reverse phase HPLC (Phenomenex Luna C₁₈ column (75×30 mm, 5 m particle size), gradient: 1-99% acetonitrile in water (5 mM HCl) over 15.0 minutes) to afford N-[2-(2,6-dimethylphenyl)-6-(1-phenylcyclopropyl)pyrimidin-4-yl]-1-methyl-pyrazole-4-sulfonamide (18.5 mg, 69%) as a yellow solid. ¹H NMR (400 MHz, Chloroform-d) δ 7.56-7.38 (m, 7H), 7.26 (s, 2H), 7.12 (d, J=7.6 Hz, 2H), 6.31 (s, 1H), 3.87 (s, 3H), 2.16 (s, 6H), 1.88-1.34 (m, 4H). ESI-MS m/z calc. 459.17288, found 460.26 (M+1)⁺; Retention time: 1.87 minutes (LC method A).

VI. Bioactivity assays
1. 3T3 assay
   a. Membrane potential optical methods for assaying F508del modulation properties of compounds

The assay utilizes fluorescent voltage sensing dyes to measure changes in membrane potential using a fluorescent plate reader (e.g., FLIPR III, Molecular Devices, Inc.) as a readout for increase in functional F508del in NIH 3T3 cells. The driving force for the response is the creation of a chloride ion gradient in conjunction with channel activation by a single liquid addition step after the cells have previously been treated with compounds and subsequently loaded with a voltage sensing dye.
b. Identification of Corrector Compounds

To identify correctors of F508del, a single-addition HTS assay format was developed. This HTS assay utilizes fluorescent voltage sensing dyes to measure changes in membrane potential on the FLIPR III as a measurement for increase in gating (conductance) of F508del in F508del NIH 3T3 cells. The F508del NIH 3T3 cell cultures were incubated with the corrector compounds at a range of concentrations for 18-24 hours at 37° C., and subsequently loaded with a redistribution dye. The driving force for the response is a Cl⁻ ion gradient in conjunction with channel activation with forskolin in a single liquid addition step using a fluorescent plate reader such as FLIPR III. The efficacy and potency of the putative F508del correctors was compared to that of the known corrector, lumacaftor, in combination with acutely added 300 nM Ivacaftor.

c. Solutions
Bath Solution #1: (in mM) NaCl 160, KCl 4.5, CaCl₂) 2, MgCl₂1, HEPES 10, pH 7.4 with NaOH.
Chloride-free bath solution: Chloride salts in Bath Solution #1 (above) are substituted with gluconate salts.

d. Cell Culture

NIH3T3 mouse fibroblasts stably expressing F508del are used for optical measurements of membrane potential. The cells are maintained at 37° C. in 5% C₀₂ and 90% humidity in Dulbecco's modified Eagle's medium supplemented with 2 mM glutamine, 10% fetal bovine serum, 1×NEAA, b-ME, 1×pen/strep, and 25 mM HEPES in 175 cm² culture flasks. For all optical assays, the cells were seeded at ˜20,000/well in 384-well matrigel-coated plates. For the correction assays, the cells are cultured at 37° C. with and without compounds for 16-24 hours.
2. Enteroid Assay

a. Solutions

Base medium (ADF+++) consisted of Advanced DMEM/Ham's F12, 2 mM Glutamax, 10 mM HEPES, 1 μg/mL penicillin/streptomycin.

Intestinal enteroid maintenance medium (IEMM) consisted of ADF+++, 1×B27 supplement, 1×N2 supplement, 1.25 mM N-acetyl cysteine, 10 mM Nicotinamide, 50 ng/mL hEGF, 10 nM Gastrin, 1 μg/mL hR-spondin-1, 100 ng/mL hNoggin, TGF-b type 1 inhibitor A-83-01, 100 μg/mL Primocin, 10 μM P38 MAPK inhibitor SB202190.

Bath 1 Buffer consisted of 1 mM MgCl₂, 160 mM NaCl, 4.5 mM KCl, 10 mM HEPES, 10 mM Glucose, 2 mM CaCl₂).

Chloride Free Buffer consisted of 1 mM Magnesium Gluconate, 2 mM Calcium Gluconate, 4.5 mM Potassium Gluconate, 160 mM Sodium Gluconate, 10 mM HEPES, 10 mM Glucose.

Bath1 Dye Solution consisted of Bath 1 Buffer, 0.04% Pluronic F127, 20 μM Methyl Oxonol, 30 μM CaCCinh-AO1, 30 μM Chicago Sky Blue.

Chloride Free Dye Solution consisted of Chloride Free Buffer, 0.04% Pluronic F127, 20 μM Methyl Oxonol, 30 μM CaCCinh-AO1, 30 μM Chicago Sky Blue.

Chloride Free Dye Stimulation Solution consisted of Chloride Free Dye Solution, 10 μM forskolin, 100 μM IBMX, and 300 nM Compound III.
b. Cell Culture

Human intestinal epithelial enteroid cells were obtained from the Hubrecht Institute for Developmental Biology and Stem Cell Research, Utrecht, The Netherlands and expanded in T-Flasks as previously described (Dekkers J F, Wiegerinck C L, de Jonge H R, Bronsveld I, Janssens H M, de Winter-de Groot K M, Brandsma A M, de Jong N W M, Bijvelds M J C, Scholte B J, Nieuwenhuis E E S, van den Brink S, Clevers H, van der Ent C K, Middendorp S and M Beekman J M. A functional CFTR assay using primary cystic fibrosis intestinal organoids. Nat Med. 2013 July;19(7):939-45.).
c. Enteroid Cell Harvesting and Seeding

Cells were recovered in cell recovery solution, collected by centrifugation at 650 rpm for 5 minutes at 4° C., resuspended in TryPLE and incubated for 5 min at 37° C. Cells were then collected by centrifugation at 650 rpm for 5 minutes at 4° C. and resuspended in IEMM containing 10 μM ROCK inhibitor (RI). The cell suspension was passed through a 40 μm cell strainer and resuspended at 1×106 cells/mL in IEMM containing 10 μM RI. Cells were seeded at 5000 cells/well into multi-well plates and incubated for overnight at 37° C., 95% humidity and 5% C₀₂ prior to assay.

d. Membrane Potential Dye, Enteroid Assay A

Enteroid cells were incubated with test compound in IEMM for 18-24 hours at 37° C., 95% humidity and 5% C₀₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added, and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 minutes. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 3 μM (S)—N-((6-aminopyridin-2-yl)sulfonyl)-6-(3-fluoro-5-isobutoxyphenyl)-2-(2,2,4- trimethylpyrrolidin-1-yl)nicotinamide, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity
e. Membrane Potential Dye, Enteroid Assay B

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added and the cells were incubated for 25 minutes at room temperature. Following dye incubation, cells were washed 3 times in Chloride Free Dye Solution. Chloride transport was initiated by addition of Chloride Free Dye Stimulation Solution and the fluorescence signal was read for 15 minutes. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 1 μM (14S)-8-[3-(2-{Dispiro[2.0.2.1]heptan-7-yl}ethoxy)-1H-pyrazol-1-yl]-12,12-dimethyl-2 k₆-thia-3,9,11,18,23-pentaazatetracyclo[17.3.1.111,14.05,10]tetracosa-1(22),5,7,9,19(23),20-hexaene-2,2,4-trione, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).
1. Biological Activity Data

The following table represent CFTR modulating activity for representative compounds of the invention generated using one or more of the assays disclosed herein (EC₅₀ : +++ is <1 μM; ++ is 1-<3 μM; + is 3-<30 μM; and ND is “not detected in this assay.” % Activity: +++ is >60%; ++ is 30-60%; + is <30%).

Lengthy table referenced here
US20230373935A1-20231123-T00001
Please refer to the end of the specification for access instructions.

### VIII. Synthesis of (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol

A. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. ¹⁹F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before fourrier transform. ¹⁹F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H₁/C₁₃/F19/P31) probe (type: 250-SB, s #23055/0020) or on a Varian 500 MHz instrument equipped with a ID PFG, 5 mm, 50-202/500 MHz probe (model/part #99337300).

Unless stated to the contrary in the following examples, final purity of compounds was determined by reversed phase UPLC using an Acquity UPLC BEH C₁₈ column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]* species obtained using a single quadrupole mass spectrometer equipped with an electrospray ionization (ESI) source capable of achieving a mass accuracy of 0.1 Da and a minimum resolution of 1000 (no units on resolution) across the detection range.

Solid-state NMR (SSNMR) spectra were recorded on a Bruker-Biospin 400 MHz wide-bore spectrometer equipped with Bruker-Biospin 4 mm HFX probe. Samples were packed into 4 mm ZrO2 rotors and spun under Magic Angle Spinning (MAS) condition with spinning speed typically set to 12.5 kHz. The proton relaxation time was measured using ¹H MAS Ti saturation recovery relaxation experiment in order to set up proper recycle delay of the ¹³C cross-polarization (CP) MAS experiment. The fluorine relaxation time was measured using ¹⁹F MAS Ti saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz.

B. Procedures for the Synthesis of Intermediates

### Intermediate 1: Preparation of methyl 3-[bis(tert-butoxycarbonyl)amino]-6-bromo-5-(trifluoromethyl)pyridine-2-carboxylate

### Step 1: Methyl 3-(benzhydrylideneamino)-5-(trifluoromethyl)pyridine-2-carboxylate

A mixture of methyl 3-chloro-5-(trifluoromethyl)pyridine-2-carboxylate (47.3 g, 197.43 mmol), diphenylmethanimine (47 g, 259.33 mmol), Xantphos (9.07 g, 15.675 mmol), and cesium carbonate (131 g, 402.06 mmol) in dioxane (800 mL) was degassed with bubbling nitrogen for 30 minutes. Pd(OAc)₂ (3.52 g, 15.679 mmol) was added and the system was purged with nitrogen three times. The reaction mixture was heated at 100° C. for 18 h. The reaction was cooled to room temperature and filtered on a pad of Celite. The cake was washed with EtOAc and solvents were evaporated under reduced pressure to give methyl 3-(benzhydrylideneamino)-5-(trifluoromethyl)pyridine-2-carboxylate (90 g, 84%) as yellow solid. ESI-MS m/z calc. 384.10855, found 385.1 (M+1)⁺; Retention time: 2.24 minutes. LCMS Method: Kinetex C₁₈ 4.6 ×50 mm 2.6 μM, 2.0 mL/min, 95% H₂O (0.1% formic acid)⁺5% acetonitrile (0.1% formic acid) to 95% acetonitrile (0.1% formic acid) gradient (2.0 min) then held at 95% acetonitrile (0.1% formic acid) for 1.0 min.

### Step 2: Methyl 3-amino-5-(trifluoromethyl)pyridine-2-carboxylate

To a suspension of methyl 3-(benzhydrylideneamino)-5-(trifluoromethyl)pyridine-2-carboxylate (65 g, 124.30 mmol) in methanol (200 mL) was added HCl (3 M in methanol) (146 mL of 3 M, 438.00 mmol). The mixture was stirred at room temperature for 1.5 hour then the solvent was removed under reduced pressure. The residue was taken up in ethyl acetate (2 L) and dichloromethane (500 mL). The organic phase was washed with 5% aqueous sodium bicarbonate solution (3×500 mL) and brine (2×500 mL), dried over anhydrous sodium sulfate, filtered and the solvent was removed under reduced pressure. The residue was triturated with heptanes (2×50 mL) and the mother liquors were discarded. The solid obtained was triturated with a mixture of dichloromethane and heptanes (1:1, 40 mL) and filtered to afford methyl 3-amino-5-(trifluoromethyl)pyridine-2-carboxylate (25.25 g, 91%) as yellow solid. ¹H NMR (300 MHz, CDCl₃) δ 8.24 (s, 1H), 7.28 (s, 1H), 5.98 (br. s, 2H), 4.00 (s, 3H) ppm. 19F NMR (282 MHz, CDCl₃) δ -63.23 (s, 3F) ppm. ESI-MS m/z calc. 220.046, found 221.1 (M+1)⁺; Retention time: 1.62 minutes. LCMS Method: Kinetex Polar C₁₈ 3.0 ×50 mm 2.6 μm, 3 min, 5-95% acetonitrile in H₂O (0.1% formic acid) 1.2 mL/min.

### Step 3: Methyl 3-amino-6-bromo-5-(trifluoromethyl)pyridine-2-carboxylate

To a solution of methyl 3-amino-5-(trifluoromethyl)pyridine-2-carboxylate (18.75 g, 80.91 mmol) in acetonitrile (300 mL) at 0° C. was added portion wise N-bromosuccinimide (18.7 g, 105.3 mmol). The mixture was stirred overnight at 25° C. Ethyl acetate (1000 mL) was added. The organic layer was washed with 10% sodium thiosulfate solution (3×200 mL) which were back extracted with ethyl acetate (2×200 mL). The combined organic extracts were washed with saturated sodium bicarbonate solution (3×200 mL), brine (200 mL), dried over sodium sulfate and concentrated in vacuo to provide methyl 3-amino-6-bromo-5-(trifluoromethyl)pyridine-2-carboxylate (25.46 g, 98%). ¹H NMR (300 MHz, CDCl₃) δ 3.93-4.03 (m, 3H), 6.01 (br. s., 2H), 7.37 (s, 1H) ppm. ¹⁹F NMR (282 MHz, CDCl₃) ppm −64.2 (s, 3F). ESI-MS m/z calc. 297.9565, found 299.0 (M+1)⁺; Retention time: 2.55 minutes. LCMS Method: Kinetex C₁₈ 4.6 ×50 mm 2.6 μM. Temp: 45° C., Flow: 2.0 mL/min, Run Time: 6 min. Mobile Phase: Initial 95% H₂O (0.1% formic acid) and 5% acetonitrile (0.1% formic acid) linear gradient to 95% acetonitrile (0.1% formic acid) for 4.0 min then held at 95% acetonitrile (0.1% formic acid) for 2.0 min.

### Step 4: Methyl 3-[bis(tert-butoxycarbonyl)amino]-6-bromo-5-(trifluoro methyl)pyridine-2-carboxylate

A mixture of methyl 3-amino-6-bromo-5-(trifluoromethyl)pyridine-2-carboxylate (5 g, 15.549 mmol), (Boc)₂₀ (11 g, 11.579 mL, 50.402 mmol), DMAP (310 mg, 2.5375 mmol) and CH₂C₁₂ (150 mL) was stirred at room temperature overnight. The reaction mixture was concentrated under reduced pressure and purification by silica gel chromatography (0-15% ethyl acetate in heptane) provided methyl 3-[bis(tert-butoxycarbonyl)amino]-6-bromo-5-(trifluoromethyl)pyridine-2-carboxylate (6.73 g, 87%) as light yellow solid. ¹H NMR (300 MHz, CDCl₃) δ 1.42 (s, 18H), 3.96 (s, 3H), 7.85 (s, 1H) ppm. ¹⁹F NMR (282 MHz, CDCl₃) δ-63.9 (s, 3F) ppm. ESI-MS m/z calc. 498.06134, Retention time: 2.34 minutes. LCMS Method: Kinetex C₁₈ 4.6 ×50 mm 2.6 μM. Temp: 45° C., Flow: 2.0 mL/min, Run Time: 3 min. Mobile Phase: Initial 95% H₂O (0.1% formic acid) and 5% acetonitrile (0.1% formic acid) linear gradient to 95% acetonitrile (0.1% formic acid) for 2.0 min then held at 95% acetonitrile (0.1% formic acid) for 1.0 min.

### Intermediate 2: Preparation of 6-bromo-3-(tert-butoxycarbonylamino)-5-(trifluoromethyl)pyridine-2-carboxylic acid

### Step 1: 6-Bromo-3-(tert-butoxycarbonylamino)-5-(trifluoromethyl)pyridine-2-carboxylic acid

To a mixture of methyl 3-[bis(tert-butoxycarbonyl)amino]-6-bromo-5-(trifluoromethyl)pyridine-2-carboxylate (247 g, 494.7 mmol) in THE (1.0 L) was added a solution of LiOH (47.2 g, 1.971 mol) in water (500 mL). The mixture was stirred at ambient temperature for 18 h affording a yellow slurry. The mixture was cooled with an ice-bath and slowly acidified with HCl (1000 mL of 2 M, 2.000 mol) keeping the reaction temperature <15° C. The mixture was diluted with heptane (1.5 L), mixed and the organic phase separated. The aqueous phase was extracted with heptane (500 mL). The combined organic phases were washed with brine, dried over MgSO₄, filtered and concentrated in vacuo. The crude oil was dissolved in heptane (600 mL), seeded and stirred at ambient temperature for 18 h affording a thick slurry. The slurry was diluted with cold heptane (500 mL) and the precipitate collected using a medium frit. The filter cake was washed with cold heptane and air dried for 1 h, then in vacuo at 45° C. for 48 h to afford 6-bromo-3-(tert-butoxycarbonylamino)-5-(trifluoromethyl)pyridine-2-carboxylic acid (158.3 g, 83%). ¹H NMR (400 MHz, DMSO-d₆) δ 10.38 (s, 1H), 9.01 (s, 1H), 1.50 (s, 9H) ppm. ESI-MS m/z calc. 383.99326, found 384.9 (M+1)⁺; Retention time: 2.55 minutes. LCMS Method Detail: Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=acetonitrile (0.035% CF₃CO₂H). Flow rate=1.2 mL/min, injection volume=1.5 μL, and column temperature=60° C.

### Intermediate 3: Preparation of 2-Benzyloxy-2-(trifluoromethyl)hex-5-enoic acid

### Step 1: Ethyl 2-hydroxy-2-(trifluoromethyl)hex-5-enoate

To a solution of ethyl 3,3,3-trifluoro-2-oxo-propanoate (25.15 g, 147.87 mmol) in Et20 (270 mL) at −78° C. was added bromo(but-3-enyl)magnesium in THE (190 mL of 0.817 M, 155.23 mmol) dropwise over a period of 1.5 h (inner temperature −72° C. to −76° C.). The mixture was stirred at −78° C. for 20 min. The dry ice-acetone bath was removed. The mixture was slowly warm to 5° C. during 1 h, added to a mixture of 1 N aqueous HCl (170 mL) and crushed ice (150 g) (pH=4). The two layers were separated. The organic layer was concentrated, and the residue was combined with aqueous phase and extracted with EtOAc (2×150 mL). The combined organic phase was washed with 5% aqueous NaHCO₃(50 mL) and brine (20 mL), dried with Na₂SO₄. The mixture was filtered and concentrated, and co-evaporated with THE (2×40 mL) to give ethyl 2-hydroxy-2-(trifluoromethyl)hex-5-enoate (37.44 g, 96%) as colorless oil. ¹H NMR (300 MHz, CDCl₃) δ 5.77 (ddt, J=17.0, 10.4, 6.4 Hz, 1H), 5.15-4.93 (m, 2H), 4.49-4.28 (m, 2H), 3.88 (s, 1H), 2.35-2.19 (m, 1H), 2.17-1.89 (m, 3H), 1.34 (t, J=7.0 Hz, 3H) ppm. ¹⁹F NMR (282 MHz, CDCl₃) δ −78.74 (s, 3F) ppm.

### Step 2: Ethyl 2-benzyloxy-2-(trifluoromethyl)hex-5-enoate

To a solution of ethyl 2-hydroxy-2-(trifluoromethyl)hex-5-enoate (24.29 g, 87.6% purity, 94.070 mmol) in DMF (120 mL) at 0° C. was added NaH (60% in mineral oil, 5.64 g, 141.01 mmol) portion-wise. The mixture was stirred at 0° C. for 10 min. Benzyl bromide (24.13 g, 141.08 mmol) and TBAI (8.68 g, 23.500 mmol) were added. The mixture was stirred at room temperature overnight. NH₄C₁ (3 g, 0.6 eq) was added. The mixture was stirred for 10 min. 30 mL of EtOAc was added, then ice-water was added (400 g). The mixture was extracted with CH₂C₁₂ and the combined organic layers were concentrated. Purification by silica gel chromatography (0-20% CH₂C₁₂ in heptanes) provided ethyl 2-benzyloxy-2-(trifluoromethyl)hex-5-enoate (26.05 g, 88%) as pink oil. ¹H NMR (300 MHz, CDCl₃) δ 1.34 (t, J=7.2 Hz, 3H), 2.00-2.19 (m, 3H), 2.22-2.38 (m, 1H), 4.33 (q, J=7.2 Hz, 2H), 4.64 (d, J=10.6 Hz, 1H), 4.84 (d, J=10.9 Hz, 1H), 4.91-5.11 (m, 2H), 5.62-5.90 (m, 1H), 7.36 (s, 5H) ppm. 19F NMR (282 MHz, CDCl₃) δ −70.5 (s, 3F) ppm. ESI-MS m/z calc. 316.12863, found 317.1 (M+1)⁺; Retention time: 2.47 minutes. LCMS Method: Kinetex C₁₈ 4.6 ×50 mm 2.6 μM. Temp: 45° C., Flow: 2.0 mL/min, Run Time: 3 min. Mobile Phase: Initial 95% H₂O (0.1% formic acid) and 5% acetonitrile (0.1% formic acid) linear gradient to 95% acetonitrile (0.1% formic acid) for 2.0 min then held at 95% acetonitrile (0.1% formic acid) for 1.0 min.

### Step 3: 2-Benzyloxy-2-(trifluoromethyl)hex-5-enoic acid

A solution of sodium hydroxide (7.86 g, 196.51 mmol) in water (60 mL) was added to a solution of ethyl 2-benzyloxy-2-(trifluoromethyl)hex-5-enoate (24.86 g, 78.593 mmol) in methanol (210 mL). The reaction was heated at 50° C. overnight. The reaction was concentrated to remove methanol, diluted with water (150 mL) and the carboxylate sodium salt was washed with heptane (1×100 mL). The aqueous solution was acidified to pH=2 with aqueous 3N solution of HCl. The carboxylic acid was extracted with dichloromethane (3×100 mL) and dried over sodium sulfate. The solution was filtered and concentrated to give 2-benzyloxy-2-(trifluoromethyl)hex-5-enoic acid (22.57 g, 97%) as pale yellow oil. ¹H NMR (300 MHz, DMSO-d₆) δ 14.31 (br. s., 1H), 7.55-7.20 (m, 5H), 5.93-5.70 (m, 1H), 5.17-4.91 (m, 2H), 4.85-4.68 (m, 1H), 4.67-4.55 (m, 1H), 2.32-1.94 (m, 4H) ppm. ¹⁹F NMR (282 MHz, DMSO-d₆) δ -70.29 (s, 3F) ppm. ESI-MS m/z calc. 288.09732, found 287.1 (M−1); Retention time: 3.1 minutes. LCMS Method: Kinetex Polar C₁₈ 3.0 ×50 mm 2.6 μm, 6 min, 5-95% acetonitrile in H₂O (0.1% formic acid) 1.2 mL/min. Intermediate 4: Preparation of (2R)-2-benzyloxy-2-(trifluoromethyl)hex-5-enoic acid

### Step 1: (2R)-2-Benzyloxy-2-(trifluoromethyl)hex-5-enoic acid; (R)-4-quinolyl-[(2S,4S)-5-vinylquinuclidin-2-yl]methanol

To a N2 purged jacketed reactor set to 20° C. was added isopropyl acetate (IPAC, 100 L, 0.173 M, 20 Vols), followed by previously melted 2-benzyloxy-2-(trifluoromethyl)hex-5-enoic acid (5.00 kg, 17.345 mol) and cinchonidine (2.553 kg, 8.67 mol) made into a slurry with minor amount of the reaction solvent. The reactor was set to ramp internal temperature to 80° C. over 1 hour, with solids going in solution upon heating to set temperature, then the solution was held at temperature for at least 10 minutes, then cooled to 70° C. held and seeded with chiral salt (50 g, 1.0% by wt). The mixture was stirred for 10 minutes, then ramped to 20° C. internal temperature over 4 hours, then held overnight at 20° C. The mixture was filtered, cake washed with isopropyl acetate (10.0 L, 2.0 vols) and dried under vacuum. The cake was then dried in vacuo (50° C., vacuum) to afford 4.7 kg of salt. The resulting solid salt was returned to the reactor by making a slurry with a portion of isopropyl acetate (94 L, 20 vol based on current salt wt), and pumped into reactor and stirred. The mixture was then heated to 80° C. internal, stirred hot slurry for at least 10 minutes, then ramped to 20° C. over 4-6 h, then stirred overnight at 20° C. The material was then filtered and cake washed with isopropyl acetate (9.4 L, 2.0 vol), pulled dry, cake scooped out and dried in vacuo (50° C., vacuum) to afford 3.1 kg of solid. The solid (3.1 kg) and isopropyl acetate (62 L, 20 vol based on salt solid wt) was slurried and added to a reactor, stirred under N2 purge and heated to 80° C. and held at temperature at least 10 minutes, then ramped to 20° C. over 4-6 hours, then stirred overnight. The mixture was filtered, cake washed with isopropyl acetate (6.2 L, 2 vol), pulled dry, scooped out and dried in vacuo (50° C., vac) to afford 2.25 kg of solid salt. The solid (2.25 kg) and isopropyl acetate (4.5 L, 20 vol based on salt solid wt) was slurried and added to a reactor, stirred under N2 purge and heated to 80° C., held at temperature at least 10 minutes, then ramped to 20° C. over 4-6 hours, then stirred overnight. The mixture was filtered, cake washed with isopropyl acetate (4.5 L, 2 vol), pulled dry, scooped out and dried in vacuo (50° C. to afford (2R)-2-benzyloxy-2-(trifluoromethyl)hex-5-enoic acid;(R)-4-quinolyl-[(2S,4S)-5-vinylquinuclidin-2-yl]methanol (1.886 kg, >98.0% ee ) as off-white to tan solid. Chiral purity was determined by Agilent 1200 HPLC instrument using Phenomenex Lux i-Amylose-3 column (3 μm, 150×4.6 mm) and a dual, isocratic gradient run 30% to 70% mobile phase B over 20.0 minutes. Mobile phase A=H₂O (0.1% CF₃CO₂H). Mobile phase B=MeOH (0.1% CF₃CO₂H). Flow rate=1.0 mL/min, injection volume=2 μL, and column temperature=30° C., sample concentration: 1 mg/mL in 60% acetonitrile/40% water.

### Step 2: (2R)-2-Benzyloxy-2-(trifluoromethyl)hex-5-enoic acid

A suspension of (2R)-2-benzyloxy-2-(trifluoromethyl)hex-5-enoic acid; (R)-4-quinolyl-[(2S,4S)-5-vinylquinuclidin-2-yl]methanol (50 g, 87.931 mmol) in ethyl acetate (500.00 mL) was treated with an aqueous solution of hydrochloric acid (200 mL of 1 M, 200.00 mmol). After stirring 15 minutes at room temperature, the two phases were separated. The aqueous phase was extracted twice with ethyl acetate (200 mL). The combined organic layer was washed with 1 N HCl (100 mL). The organic layer was dried over sodium sulfate, filtered and concentrated. The material was dried over high vacuum overnight to give (2R)-2-benzyloxy-2-(trifluoromethyl)hex-5-enoic acid (26.18 g, 96%) as pale brown oil. ¹H NMR (400 MHz, CDCl₃) δ 7.46-7.31 (m, 5H), 5.88-5.73 (m, 1H), 5.15-4.99 (m, 2H), 4.88 (d, J=10.3 Hz, 1H), 4.70 (d, J=10.3 Hz, 1H), 2.37-2.12 (m, 4H) ppm. ¹⁹F NMR (377 MHz, CDCl₃) δ-71.63 (br s, 3F) ppm. ESI-MS m/z calc. 288.0973, found 287.0 (M−1); Retention time: 2.15 minutes. LCMS Method: Kinetex Polar C₁₈ 3.0 ×50 mm 2.6 μm, 3 min, 5-95% acetonitrile in H₂O (0.1% formic acid) 1.2 mL/min.

### Intermediate 5: Preparation of (2R)-2-benzyloxy-2-(trifluoromethyl)hex-5-enehydrazide

### Step 1: tert-Butyl N-[[(2R)-2-benzyloxy-2-(trifluoromethyl)hex-5-enoyl]amino]carbamate

To a solution of (2R)-2-benzyloxy-2-(trifluoromethyl)hex-5-enoic acid (365 g, 1.266 mol) in DMF (2 L) was added HATU (612 g, 1.610 mol) and DIEA (450 mL, 2.584 mol) and the mixture was stirred at ambient temperature for 10 min. To the mixture was added tert-butyl N-aminocarbamate (200 g, 1.513 mol) (slight exotherm upon addition) and the mixture was stirred at ambient temperature for 16 h. The reaction was poured into ice water (5 L). The resultant precipitate was collected by filtration and washed with water. The solid was dissolved in EtOAc (2 L) and washed with brine. The organic phase was dried over MgSO₄, filtered and concentrated in vacuo. The oil was diluted with EtOAc (500 mL) followed by heptane (3 L) and stirred at ambient temperature for several hours affording a thick slurry. The slurry was diluted with additional heptane and filtered to collect fluffy white solid (343 g). The filtrate was concentrated and purification by silica gel chromatography (0-40% EtOAc/hexanes) provided tert-butyl N-[[(2R)-2-benzyloxy-2-(trifluoromethyl)hex-5-enoyl]amino]carbamate (464 g, 91%, combined with product from crystallization). ESI-MS m/z calc. 402.17664, found 303.0 (M+1-Boc)*; Retention time: 2.68 minutes. Final purity was determined by reversed phase UPLC using an Acquity UPLC BEH C₁₈ column (50×2.1 mm, 1.7 m particle) made by Waters (pn: 186002350) and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate =1.2 mL/min, injection volume=1.5 μL, and column temperature=60° C.

### Step 2: (2R)-2-Benzyloxy-2-(trifluoromethyl)hex-5-enehydrazide

To a solution of tert-butyl N-[[(2R)-2-benzyloxy-2-(trifluoromethyl)hex-5-enoyl]amino]carbamate (464 g, 1.153 mol) in DCM (1.25 L) and was added HCl (925 mL of 4 M, 3.700 mol) and the mixture stirred at ambient temperature for 20 h. The mixture was concentrated in vacuo removing most of the DCM. The mixture was diluted with isopropyl acetate (1 L) and basified to pH=6 with NaOH (140 g of 50% w/w, 1.750 mol) in 1 L of ice water. The organic phase was separated and washed with 1 L of brine and the combined aqueous phases were extracted with isopropyl acetate (1 L). The combined organic phases were dried over MgSO₄, filtered and concentrated in vacuo affording a dark yellow oil of (2R)-2-benzyloxy-2-(trifluoromethyl)hex-5-enehydrazide (358 g, quant.). ¹H NMR (400 MHz, CDCl₃) δ 8.02 (s, 1H), 7.44-7.29 (m, 5H), 5.81 (ddt, J=16.8, 10.1, 6.4 Hz, 1H), 5.13-4.93 (m, 2H), 4.75 (dd, J=10.5, 1.5 Hz, 1H), 4.61 (d, J=10.5 Hz, 1H), 3.78 (s, 2H), 2.43 (ddd, J=14.3, 11.0, 5.9 Hz, 1H), 2.26-1.95 (m, 3H) ppm. ESI-MS m/z calc. 302.1242, found 303.0 (M+1)⁺; Retention time: 2.0 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate =1.2 mL/min, injection volume=1.5 μL, and column temperature=60° C.

### Intermediate 6: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

### Step 1: tert-Butyl N-[2-[[[(2R)-2-benzyloxy-2-(trifluoromethyl)hex-5-enoyl]amino]carbamoyl]-6-bromo-5-(trifluoromethyl)-3-pyridyl]carbamate

To a mixture of 6-bromo-3-(tert-butoxycarbonylamino)-5-(trifluoromethyl)pyridine-2-carboxylic acid (304 g, 789.3 mmol) and (2R)-2-benzyloxy-2-(trifluoromethyl)hex-5-enehydrazide (270 g, 893.2 mmol) in EtOAc (2.25 L) at ambient temperature was added DIEA (425 mL, 2.440 mol). To the mixture was slowly added T3P (622 g of 50% w/w, 977.4 mmol) using an ice-water bath to keep the temperature <35° C. (temperature rose to 34° C.) and the reaction mixture was stirred at ambient temperature for 18 h. Added additional DIEA (100 mL, 574.1 mmol) and T3P (95 g, 298.6 mmol) and stirred at ambient temperature for 2 days. Starting material was still observed and an additional T3P (252 g, 792 mmol) was added and stirred for 5 days. The reaction was quenched with the slow addition of water (2.5 L) and the mixture stirred for 30 min. The organic phase was separated, and the aqueous phase extracted with EtOAc (2 L). The combined organic phases were washed with brine, dried over MgSO₄, filtered and concentrated in vacuo. The crude product was dissolved in MTBE (300 mL) and diluted with heptane (3 L), the mixture stirred at ambient temperature for 12 h affording a light yellow slurry. The slurry was filtered, and the resultant solid was air dried for 2 h, then in vacuo at 40° C. for 48 h. The filtrate was concentrated in vacuo and purified by silica gel chromatography (0-20% EtOAc/hexanes) and combined with material obtained from crystallization providing tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (433 g, 82%). ¹H NMR (400 MHz, DMSO) δ 11.07 (s, 1H), 10.91 (s, 1H), 10.32 (s, 1H), 9.15 (s, 1H), 7.53-7.45 (m, 2H), 7.45-7.28 (m, 3H), 5.87 (ddt, J=17.0, 10.2, 5.1 Hz, 1H), 5.09 (dq, J=17.1, 1.3 Hz, 1H), 5.02 (dd, J=10.3, 1.9 Hz, 1H), 4.84 (q, J=11.3 Hz, 2H), 2.37-2.13 (m, 4H), 1.49 (s, 9H) ppm. ESI-MS m/z calc. 668.1069, found 669.0 (M+1)⁺; Retention time: 3.55 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### Step 2: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

To a solution of tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (240 g, 358.5 mmol) in anhydrous acetonitrile (1.5 L) under nitrogen was added DIEA (230 mL, 1.320 mol) and the orange solution heated to 70° C. To the mixture was added p-toluenesulfonyl chloride (80.5 g, 422.2 mmol) in 3 equal portions over 1 h. The mixture was stirred at 70° C. for 9 h then additional p-toluenesulfonyl chloride (6.5 g, 34.09 mmol) was added. The mixture was stirred for a total of 24 h then allowed to cool to ambient temperature. Acetonitrile was removed in vacuo affording a dark orange oil which was diluted with EtOAc (1.5 L) and water (1.5 L). The organic phase was separated and washed with 500 mL of 1M HCl, 500 mL of brine, dried over MgSO₄, filtered and concentrated in vacuo. Purification by silica gel chromatography (0-20% EtOAc/hexanes) provided tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (200 g, 86%). ¹H NMR (400 MHz, DMSO) δ 10.11 (s, 1H), 9.10 (s, 1H), 7.55-7.48 (m, 2H), 7.47-7.28 (m, 3H), 5.87 (ddt, J=16.7, 10.2, 6.4 Hz, 1H), 5.11 (dt, J=17.2, 1.7 Hz, 1H), 5.01 (dt, J=10.2, 1.5 Hz, 1H), 4.74 (d, J=10.6 Hz, 1H), 4.65 (d, J=10.6 Hz, 1H), 2.55-2.42 (m, 2H), 2.30 (qd, J=11.3, 10.3, 6.9 Hz, 2H), 1.52 (s, 9H) ppm. ESI-MS m/z calc. 650.0963, found 650.0 (M+1)⁺; Retention time: 3.78 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05 % CF₃CO₂H). Mobile phase B═CH₃CN (0.035% CF₃CO₂H). Flow rate=1.2 mL/min, injection volume=1.5 μL, and column temperature=60° C.

### Intermediate 7: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

To a solution of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (222 g, 340.8 mmol) in MTBE (1.333 L) was added DIPEA (65.3 mL, 374.9 mmol) followed DMAP (2.09 g, 17.11 mmol). Added a solution of di-tert-butyl dicarbonate (111.6 g, 511.3 mmol) in MTBE (250 mL) over approx. 8 minutes, and the resulting mixture was stirred for additional 30 min. Added 1 L of water and separated the layers. The organic layer was washed with KHSO₄ (886 mL of 0.5 M, 443.0 mmol), 300 mL brine, dried with MgSO₄ and most (>95%) of the MTBE was evaporated by rotary evaporation at 45° C., leaving a thick oil. Added 1.125 L of heptane, spun in the 45° C. rotovap bath until dissolved, then evaporated out 325 mL of solvent by rotary evaporation. The rotovap bath temp was allowed to drop to room temperature and product started crystallizing out during the evaporation. Then put the flask in a −20° C. freezer overnight. The resultant solid was filtered and washed with cold heptane and dried at room temperature for 3 days to give tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (240.8 g, 94%). ¹H NMR (400 MHz, Chloroform-d) δ 7.95 (s, 1H), 7.52-7.45 (m, 2H), 7.44-7.36 (m, 2H), 7.36-7.29 (m, 1H), 5.83-5.67 (m, 1H), 5.08-5.00 (m, 1H), 5.00-4.94 (m, 1H), 4.79 (d, J=10.4 Hz, 1H), 4.64 (d, J=10.4 Hz, 1H), 2.57-2.26 (m, 3H), 2.26-2.12 (m, 1H), 1.41 (s, 18H) ppm. ESI-MS m/z calc. 750.14874, found 751.1 (M+1)⁺; Retention time: 3.76 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### Intermediate 8: Preparation of tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (280 g, 372.6 mmol) was dissolved in DMSO (1.82 L) (yellow solution) and treated with cesium acetate (215 g, 1.120 mol) under stirring at room temperature. The yellow suspension was heated at 80° C. for 5 h. The reaction mixture was cooled to room temperature and added to a stirred cold emulsion of water (5.5 L) with 1 kg ammonium chloride dissolved in it and a 1:1 mixture of MTBE and heptane (2 L) (in 20 L). The phases were separated and the organic phase washed water (3×3 L) and with brine (1×2.5 L). The organic phase was dried with MgSO₄, filtered and concentrated under reduced pressure. The resultant yellow solution was diluted with heptane (˜1 L) and seeded with tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate and stirred on the rotavap at 100 mbar pressure at room temperature for 1.5 h. The solid mass was stirred mechanically for 2 h at room temperature, resultant thick fine suspension was filtered, washed with dry ice cold heptane and dried under vacuum at 45° C. with a nitrogen bleed for 16 h to give tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (220 g, 85%) as an off white solid. ¹H NMR (400 MHz, DMSO-d₆) δ 13.28 (s, 1H), 8.43 (s, 1H), 7.58-7.26 (m, 5H), 5.85 (ddt, J=16.8, 10.3, 6.5 Hz, 1H), 5.10 (dq, J=17.2, 1.6 Hz, 1H), 5.01 (dq, J=10.2, 1.3 Hz, 1H), 4.76 (d, J=11.0 Hz, 1H), 4.65 (d, J=11.0 Hz, 1H), 2.55 (dd, J=9.6, 5.2 Hz, 2H), 2.23 (td, J=13.2, 10.0, 5.7 Hz, 2H), 1.27 (d, J=3.8 Hz, 18H) ppm. ESI-MS m/z calc. 688.23315, found 689.0 (M+1)⁺; Retention time: 3.32 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B ═CH₃CN (0.035% CF₃CO₂H). Flow rate=1.2 mL/min, injection volume=1.5 μL, and column temperature=60° C.

### C. Preparation of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

### Step 1: tert-Butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate

Dissolved tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (159.3 g, 231.3 mmol) and triphenylphosphine (72.9 g, 277.9 mmol) in toluene (1 L), then added (2S)-pent-4-en-2-ol (28.7 mL, 278.9 mmol). Heated this mixture to 45° C., then added DIAD (58.3 mL, 296.1 mmol) (exotherm) slowly over 40 min. For the next approximately 2 h, the mixture was cooled to room temperature. During this cooling period, after the first 10 minutes, triphenylphosphine (6.07 g, 23.14 mmol) was added. After a further 1 h, additional triphenylphosphine (3.04 g, 11.59 mmol) was added. After a further 23 min, DIAD (2.24 mL, 11.57 mmol) was added. After the ˜2 h cooling to room temperature period, the mixture was cooled to 15° C., and seed crystals of DIAD-triphenylphosphine oxide complex were added which caused precipitation to occur, then added 1000 mL heptane. Stored the mixture at −20° C. for 3 days. Filtered out and discarded the precipitate and concentrated the filtrate to give a red residue/oil. Dissolved the residue in 613 mL heptane at 45° C., then cooled to 0° C., seeded with DIAD-triphenylphosphine oxide complex, stirred at 0° C. for 30 min, then filtered the solution. The filtrate was concentrated to a smaller volume, then loaded onto a 1.5 kg silica gel column (column volume=2400 mL, flow rate=600 mL/min). Ran a gradient of 1% to 6% EtOAc in hexanes over 32 minutes (8 column volumes), then held at 6% EtOAc in hexanes until the product finished eluting which gave tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (163.5 g, 93%). ¹H NMR (400 MHz, Chloroform-d) δ 7.82 (s, 1H), 7.43-7.27 (m, 5H), 5.88-5.69 (m, 2H), 5.35 (h, J=6.2 Hz, 1H), 5.16-4.94 (m, 4H), 4.81 (d, J=10.7 Hz, 1H), 4.63 (d, J=10.7 Hz, 1H), 2.58-2.15 (m, 6H), 1.42 (s, 18H), 1.36 (d, J=6.2 Hz, 3H) ppm. ESI-MS m/z calc. 756.2958, found 757.3 (M+1)⁺; Retention time: 4.0 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 2: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture)

The following reaction was run, split equally between two, 12 L reaction flasks run in parallel. Mechanical stirring was employed, and reactions were subjected to a constant nitrogen gas purge using a course porosity gas dispersion tube. To each flask was added tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (54 g, 71.36 mmol in each flask) dissolved in DCE (8 L in each flask) and both flasks were strongly purged with nitrogen at room temperature. Both flasks were heated to 62° C. and Grubbs 1^{st}Generation Catalyst (9 g, 10.94 mmol in each flask) was added to each reaction and stirred at 400 rpm while setting an internal temperature control to 75° C. with strong nitrogen purging (both reactions reached ˜75° C. after approximately 20 min). After 5 h 15 min, the internal temperature control was set to 45° C. After approximately 2 h, 2-sulfanylpyridine-3-carboxylic acid (11 g, 70.89 mmol in each flask) was added to each flask followed by triethylamine (10 mL, 71.75 mmol in each flask). On completion of addition, the nitrogen purge was turned off and both reaction flasks were stirred at 45° C. open to air overnight. The reactions were then removed from heat and 130 g of silica gel was added to each reaction and each was stirred at room temperature. After approximately 2 h, the green mixtures were combined and filtered over Celite then concentrated by rotary evaporation at 43° C. The obtained residue was dissolved in dichloromethane/heptane 1:1 (400 mL) and the formed orange solid was removed by filtration. The greenish mother liquor was evaporated to give 115.5 g of a green foam. Dissolved this material in 500 mL of 1:1 dichloromethane/hexanes then loaded onto a 3 kg silica gel column (column volume=4800 mL, flow rate=900 mL/min). Ran a gradient of 2% to 9% EtOAc in hexanes over 43 minutes (8 column volumes), then ran at 9% EtOAc until the product finished eluting giving 77.8 g of impure product. This material was co-evaporated with methanol (˜500 mL) then diluted with methanol (200 mL) to give 234.5 g of a methanolic solution, which was halved and each half was purified by reverse phase chromatography (3.8 kg C₁₈ column, column volume=3300 mL, flow rate=375 mL/min, loaded as solution in methanol). Ran the column at 55% acetonitrile for ˜5 minutes (0.5 column volumes), then at a gradient of 55% to 100% acetonitrile in water over ˜170 minutes (19-20 column volumes), then held at 100% acetonitrile until the product and impurities finished eluting. Clean product fractions from both columns were combined and concentrated by rotary evaporation then transferred with ethanol into 5 L flask, evaporated and carefully dried (becomes a foam) to give as a mixture of olefin isomers, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (55.5 g, 53%). ESI-MS m/z calc. 728.26447, found 729.0 (M+1)⁺; Retention time: 3.82 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 3: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (11.7 g, 16.06 mmol) was dissolved in stirring ethanol (230 mL) and cycled the flask 3 times vacuum/nitrogen and treated with 10% Pd/C (50% water wet, 2.2 g of 5% w/w, 1.034 mmol). The mixture was cycled 3 times between vacuum/nitrogen and 3 times between vacuum/hydrogen. The mixture was then stirred strongly under hydrogen (balloon) for 7.5 h. The catalyst was removed by filtration, replaced with fresh 10% Pd/C (50% water wet, 2.2 g of 5% w/w, 1.034 mmol) and stirred vigorously under hydrogen (balloon) overnight. Then, the catalyst was removed again by filtration, the filtrate evaporated and the residue (11.3 g, 1 g set aside) was dissolved in ethanol (230 mL) charged with fresh 10% Pd/C (50% water wet, 2.2 g of 5% w/w, 1.034 mmol) and stirred vigorously under hydrogen (balloon) for 6 h, recharged again with fresh 10% Pd/C (50% water wet, 2.2 g of 5% w/w, 1.034 mmol) and stirred vigorously under hydrogen (balloon) overnight. The catalyst was removed by filtration and the filtrate was evaporated (10 g of residue obtained). This crude material (10 g+1 g set aside above) was purified by silica gel chromatography (330 g column, liquid load in dichloromethane) with a linear gradient of 0% to 15% ethyl acetate in hexane until the product eluted followed by 15% to 100% ethyl acetate in hexane to giving, as a colorless foam, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (9.1 g, 78%). ESI-MS m/z calc. 730.2801, found 731.0 (M+1)⁺; Retention time: 3.89 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 4: (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (8.6 g, 11.77 mmol) was dissolved in ethanol (172 mL) then the flask was cycled 3 times between vacuum/nitrogen. Treated the mixture with 10% Pd/C (50% water wet, 1.8 g of 5 % w/w, 0.8457 mmol) then cycled 3 times between vacuum/nitrogen and 3 times between vacuum/hydrogen and then stirred vigorously under hydrogen (balloon) at room temperature for 18 h. The mixture was cycled 3 times between vacuum/nitrogen, filtered over Celite washing with ethanol and then the filtrate was evaporated to give 7.3 g of tert-butyl N-tert-butoxycarbonyl-N-[(6R,12R)-6-hydroxy-12-methyl-6,15-bis(trifluoromethyl)-13,19-dioxa-3,4,18-triazatricyclo[12.3.1.12,5] nonadeca-1(18),2,4,14,16-pentaen-17-yl]carbamate an off-white solid. 1H NMR and MS confirmed the expected product. CFTR modulatory activity was confirmed using a standard Ussing Chamber Assay for CFTR potentiator activity.

### OTHER EMBODIMENTS

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

LENGTHY TABLES
The patent application contains a lengthy table section. A copy of the table is available in electronic form from the USPTO web site (https://seqdata.uspto.gov/?pageRequest=docDetail&DocID=US20230373935A1). An electronic copy of the table will also be available from the USPTO upon request and payment of the fee set forth in 37 CFR 1.19(b)(3).

1. A compound selected from Formula I: 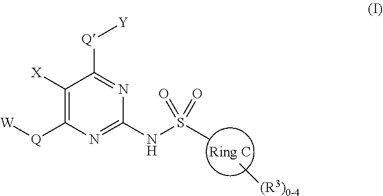
or a tautomer thereof, a deuterated derivative of the compound or tautomer, or a pharmaceutically acceptable salt of any of the foregoing, wherein
Q is absent or is oxygen
W is selected from
—H;
halogen;
—CN;
—C₁₋₈ alkyl;
—C₁₋₈ alkoxy optionally substituted with ═O;
—C₂₋₄ alkenyl;
—C₃₋₄ alkynyl; and
—NH₂ optionally substituted with 1-2 groups selected from C₁₋₆ alkyl;
or W is 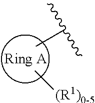
wherein:
Ring A is selected from:
—C₃₋₆ cycloalkyl,
C₅₋₁₀ aryl,
5-10 membered heterocyclyl, and
5-10 membered heteroaryl;
Each R¹ is independently selected from:
-halogen;
—OH;
—CN;
—C₁₋₆ alkyl optionally substituted with 1-3 groups selected from N(CH₃)₂, OH, ═O, halogen, C₃ cycloalkyl, C₁₋₄ alkoxy, NH₂ (optionally substituted with 1-2 groups independently selected from —C₁₋₄ alkyl, and —C(O)C₁₋₃ alkyl);
—C₁₋₆ alkenyl;
—C₁₋₈ alkoxy optionally substituted with 1-3 groups independently selected from halogen, —CN, —OH, ═O, —COOH, —C₃₋₆ cycloalkyl;
—NH₂ optionally substituted with 1-2 groups independently selected from CH₃, —S(O)₂CH₃, —C(O)C₁₋₄ alkyl;
—SR⁴
—S(O)R⁴
—S(O)₂R⁴
wherein each R⁴ is independently selected from C₁₋₃ alkyl;
—C₃₋₄ cycloalkyl optionally substituted with C(O)NH₂, C₁₋₃ alkyl;
—5-6 membered heterocyclyl optionally substituted with C₁₋₃ alkyl, CF₃;
-phenyl; and
5-6 membered heteroaryl optionally substituted with CH₃;
X is selected from:
hydrogen,
—C₁₋₆ alkyl optionally substituted with 1-5 groups selected from COOH, halogen;
—C₃₋₆ alkenyl;
—C₁₋₆ alkoxy;
—C₃₋₆ cycloalkyl;
—CN;
halogen;
phenyl optionally substituted with 1-2 groups independently selected from halogen, CN, C₁₋₃ alkoxy, C₁₋₃ alkyl; and
—O-phenyl;
Q′ is absent (i.e., Y is attached directly to the pyrimidine core), or is selected from:
—CH₂— optionally substituted with —CN, ═O, or —OH;
—NH— optionally substituted with phenyl; and
—S— optionally substituted with 1-2=0;
Y is selected from:
—H;
—C₂₋₄ alkynyl;
—C₁₋₈ alkoxy optionally substituted with 1-3 independently selected from —OH, NHC(O)CH₃;
—C₁₋₈ alkyl optionally substituted with 1-3 independently selected from:
—OH;
—CN,
halogen;
—NH₂ optionally substituted with 1-2 groups independently selected from —C(O)C₁₋₄ alkoxy, —C(O)C₁₋₃ alkyl, —CH₂-phenyl, C₁₋₈ alkyl (optionally further substituted with OH);
—C₃₋₆ cyclic alkyl;
—C₁₋₄ alkoxy
-phenyl optionally substituted with C₁₋₄ alkyl;
—5-6 membered heterocyclyl optionally substituted with 1-2 groups independently selected from ═O, OH;
5-6 membered heteroaryl;
or Y is 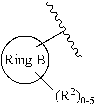
wherein:
Ring B is
—C₅₋₆ aryl;
5-10-membered heteroaryl,
4-10-membered heterocyclyl,
5-10 membered cycloalkyl,
Each R² is independently selected from:
halogen;
—CN;
—OH;
═O;
—C₁₋₈ alkyl optionally substituted with 1-5 groups independently selected from:
═O;
—OH;
—CN;
halogen;
—NH₂ optionally substituted with 1-2 groups independently selected from C₁₋₄ alkyl (optionally substituted with ═O, —CN, —OH, 3-6 membered heterocycyl (optionally substituted with CH₃), C₁₋₆ alkoxy (optionally substituted with ═O), —C₃₋₆ cyclic alkyl;
—C₁₋₄ alkoxy;
—C₃₋₁₀ cyclic alkyl optionally substituted with 1-4 groups selected from ═O, OH, CH₃, CF₃, C₆-cycloalkyl, cyano, NHC(O)C₁₋₆ alkoxy, phenyl (optionally substituted with 1-2 groups selected from halogen, —C₁₋₃ alkyl, C₁₋₃ alkoxy), —C₆ cyclic alkyl);
—C₆₋₁₀ aryl optionally substituted with 1-3 groups independently selected from halogen, —OH, —C₁₋₄ alkyl (optionally substituted with 1-3 F, —OH, —CN, ═O), —C₁₋₄ alkoxy (optionally substituted with ═O), —CN, —NH₂, S—C₁₋₃ alkyl, 5-6 membered heteroaryl;
4-10 membered heterocyclyl optionally substituted with 1-3 groups selected from ═O, OH, C₁₋₄ alkyl (optionally substituted with 1-3 F), —C(O)C₁₋₃ alkyl, —C(O)C₁₋₄ alkoxy, phenyl (optionally substituted with CH₃);
5-10 membered heteroaryl optionally substituted with 1-2 groups selected from ═O, OH, F, C₁, C₁₋₄ alkyl (optionally substituted with 1-3 F), C₁₋₄ alkoxy, —C(O)CH₃, phenyl, 5 membered heteroaryl, NH₂ (optionally substituted with 1-2 groups independently selected from C₁₋₄ alkyl, —C(O)C₁₋₄ alkoxy);
—SCH₃;
—S-phenyl optionally substituted with C₁₋₃ alkyl; and
—P(O)(CH₃)₂;
—C₄₋₆ alkynl optionally substituted with ═O;
—C₁₋₈ alkoxy optionally substituted with 1-3 groups selected from halogen, ═O, —NH₂ (optionally substituted with 1-2 groups independently selected from C₁₋₃ alkyl), phenyl (optionally substituted with 1-2 groups selected from halogen ═O, CH₃, —OCH₃, NHC(O)C₁₋₄ alkoxy);
—NH₂ optionally substituted with 1-2 groups independently selected from:
—C₁₋₆ alkyl optionally substituted with 1-2 groups selected from ═O, —OH, —NH₂, —N(CH₃)₂, —NHCH₃, —S(O)₂CH₃, C₁₋₄ alkoxy, and C₆ cycloalkyl;
—C₁₋₆ alkoxy optionally substituted with ═O;
—C₂₋₃ alkynyl;
—5-6 membered heterocyclyl optionally substituted with 1-2 groups independently selected from ═O, —C₁₋₃ alkyl);
—C₅₋₆ cycloalkyl optionally substituted with COOH;
—S(O)₂ optionally substituted with —C₁₋₃ alkyl (optionally substituted with —N(CH₃)₂),
a cyclic or bridged ring selected from -phenyl, —C₃₋₆ cyclic alkyl, 4-8 membered heterocyclyl, and 6 membered heteroaryl each of which may be optionally substituted with 1-3 groups independently selected from:
halogen;
—OH;
═O;
—NH₂ optionally substituted with 1-2 groups independently selected from C(O)C₁₋₄ alkoxy;
—C₁₋₆ alkyl optionally substituted with a group selected from 1-3 F, —OH, ═O, —NH₂ (optionally substituted with 1-2 groups independently selected from —C₁₋₆ alkoxy);
—C₁₋₆ alkoxy optionally substituted with ═O; and
—C₃ cycloalkyl;
Ring C is selected from
—C₃₋₇ cyclic alkyl;
6-membered heterocyclyl;
—C₅₋₆ aryl; and
5-10 membered heteroaryl;
Each R³ is independently selected from
hydrogen
halogen;
—CN;
═OH;
═O;
—C₁₋₆ alkyl optionally substituted with 1-3 groups independently selected from —OH, ═O, halogen, -phenyl, —NH₂ (optionally substituted with 1-2 groups independently selected from —H, —C₁₋₃ alkyl (optionally substituted with C₆ cycloalkyl, phenyl));
—C₁₋₄ alkoxy (optionally substituted with 1-3 groups independently selected from ═O and F),
—NH₂ optionally substituted with 1-2 groups independently selected from ═O, —O⁻, —C(O)CH₃, —CH₃),
6 membered heterocyclyl (optionally substituted with 1-2 F), and
—SO₂CH₃;
wherein W and Y cannot both be hydrogen.

2. The compound, tautomer, deuterated derivative, or pharmaceutically acceptable salt according to claim 1, selected from compounds of Formula Ia: 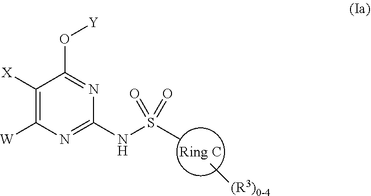
or a tautomer thereof, a deuterated derivative of the compound or tautomer, or a pharmaceutically acceptable salt of any of the foregoing, wherein variables W, X, Y, Ring C, and R³ are as defined in claim 1.

3. The compound, tautomer, deuterated derivative, or pharmaceutically acceptable salt according to claim 1, selected from compounds of Formula Ib: 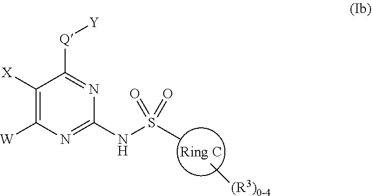
or a tautomer thereof, a deuterated derivative of the compound or tautomer, or a pharmaceutically acceptable salt of any of the foregoing, wherein
Q′ is selected from CH₂ (optionally substituted with —CN, ═O, —OH), —NH (optionally substituted with phenyl, CH₃), or S (optionally substituted with 1-2 ═O); and
variables W, X, Y, Ring C, and R³ are as defined in claim 1.

4. The compound, tautomer, deuterated derivative, or pharmaceutically acceptable salt according to claim 1, selected from compounds of Formula Ic: 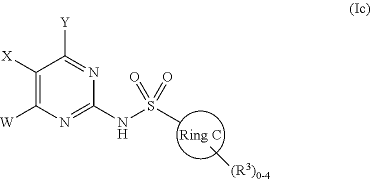
or a tautomer thereof, a deuterated derivative of the compound or tautomer, or a pharmaceutically acceptable salt of any of the foregoing, wherein variables W, X, Y, Ring C, and R³ are as defined in claim 1.

5. The compound, tautomer, deuterated derivative, or pharmaceutically acceptable salt according to claim 1, selected from compounds of Formula Id: 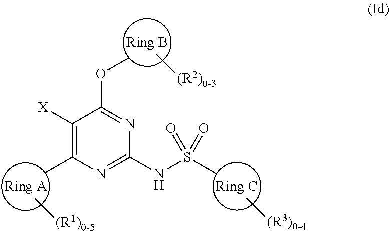
or a tautomer thereof, a deuterated derivative of the compound or tautomer, or a pharmaceutically acceptable salt of any of the foregoing, wherein variables X, R¹, R², and R³, Rings A, B, and C are as defined in claim 1.

6. The compound, tautomer, deuterated derivative, or pharmaceutically acceptable salt according to any one of claims 1-5, wherein X is hydrogen.

7. The compound, tautomer, deuterated derivative, or pharmaceutically acceptable salt according to any one of claims 1-6, wherein Rings A, B, and C are selected from optionally substituted —C₅₋₆ aryl and optionally substituted 5-10 membered heteroaryl.

8. The compound, tautomer, deuterated derivative, or pharmaceutically acceptable salt according to any one of claims 1-7, wherein Ring A is phenyl optionally substituted with 1-3 methyl groups.

9. The compound, tautomer, deuterated derivative, or pharmaceutically acceptable salt according to any one of claims 1-8, wherein Ring C is optionally substituted phenyl or optionally substituted pyrazole.

10. The compound, tautomer, deuterated derivative, or pharmaceutically acceptable salt according to any one of claims 1-9, wherein Ring C is phenyl substituted with NH₂ or pyrazole substituted with methyl.

11. The compound, tautomer, deuterated derivative, or pharmaceutically acceptable salt according to any one of claims 1-9, wherein Rings A, B, and C are optionally substituted phenyl.

12. The compound, tautomer, deuterated derivative, or pharmaceutically acceptable salt according to any one of claims 1-10, wherein at least one R² is selected from optionally substituted C₄, C₅, or C₆ cyclic alkyl and optionally substituted phenyl.

13. The compound, tautomer, deuterated derivative, or pharmaceutically acceptable salt according to any one of claims 1-11, wherein at least one R² is a substituted or unsubstituted heterocyclyl selected from:

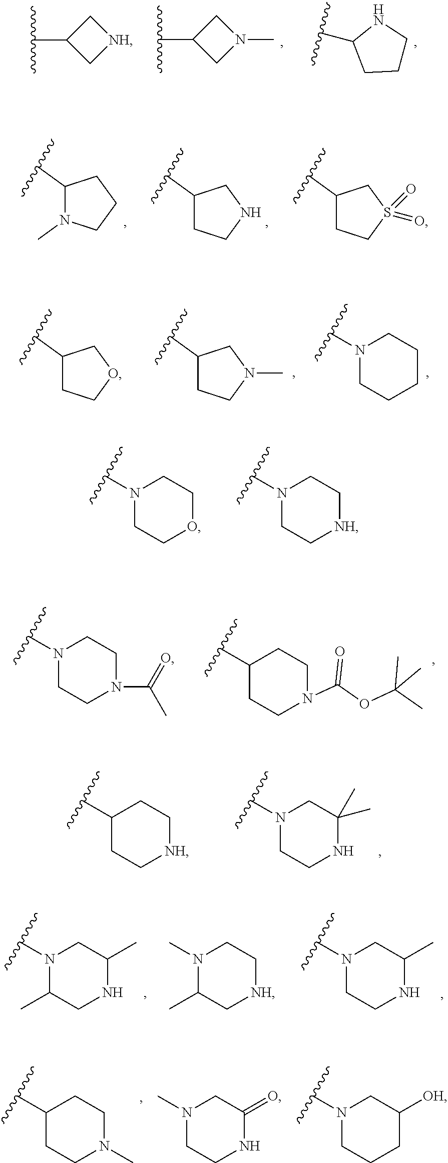

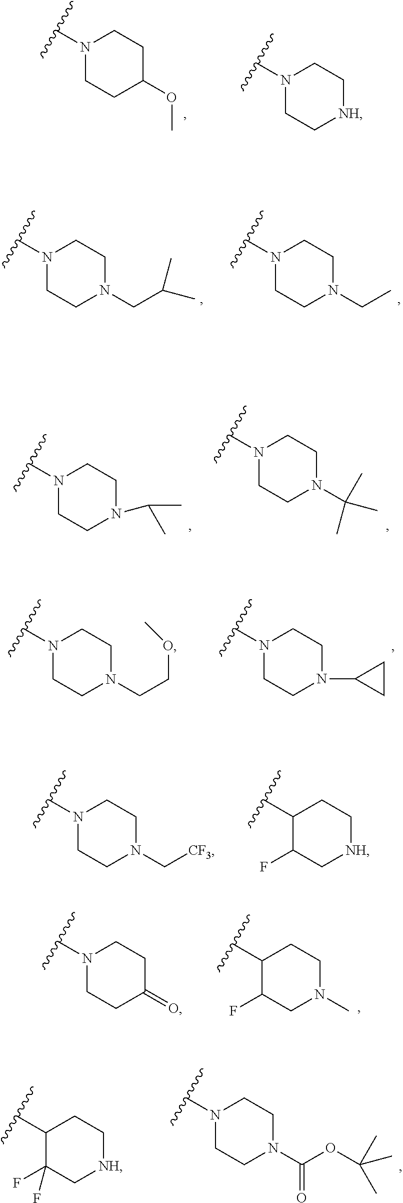

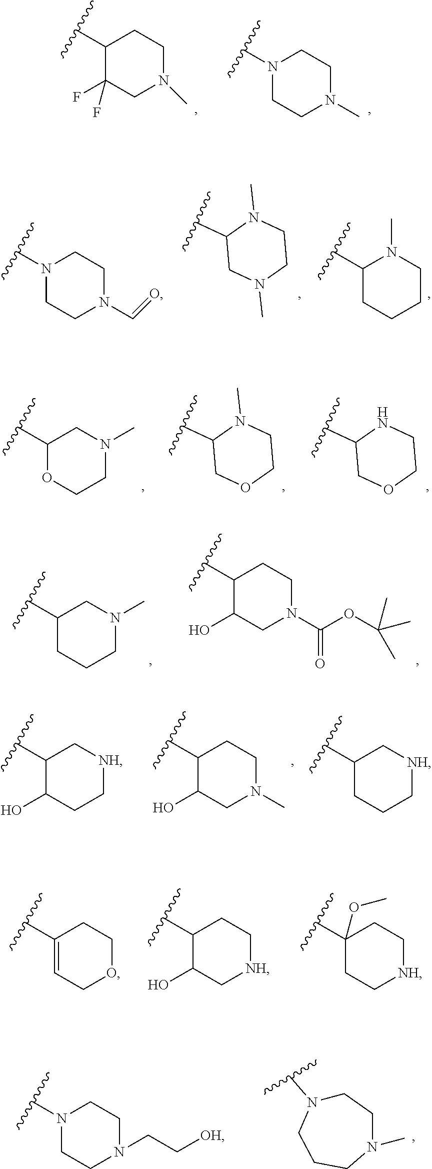

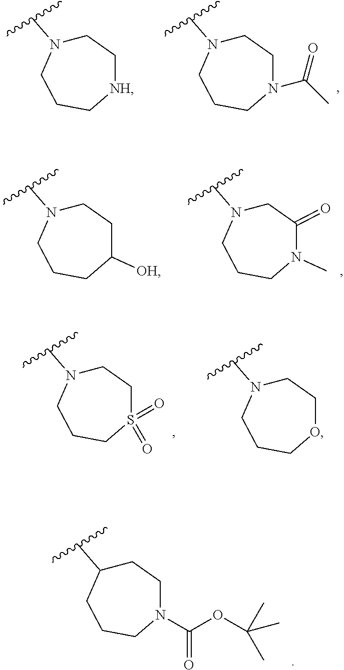

14. The compound, tautomer, deuterated derivative, or pharmaceutically acceptable salt according to any one of claims 1-6, wherein Ring A is a C₃₋₆ cycloalkyl substituted with (R¹)_{0-5.}

15. The compound, tautomer, deuterated derivative, or pharmaceutically acceptable salt according to any one of claims 1-6, wherein Ring B is a C₅₋₁₀ cycloalkyl substituted with (R²)_{0-3.}

16. The compound, tautomer, deuterated derivative, or pharmaceutically acceptable salt according to any one of claims 1-6, wherein 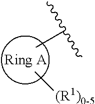
is selected from:

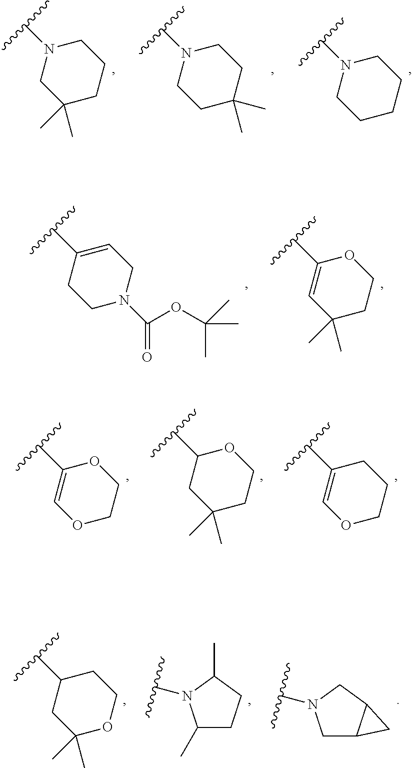

17. The compound, tautomer, deuterated derivative, or pharmaceutically acceptable salt according to any one of claims 1-9, wherein Ring B is a heteroaryl or a heterocyclyl substituted with (R²)_{0-3,} wherein the heteroaryl or heterocyclyl is is selected from

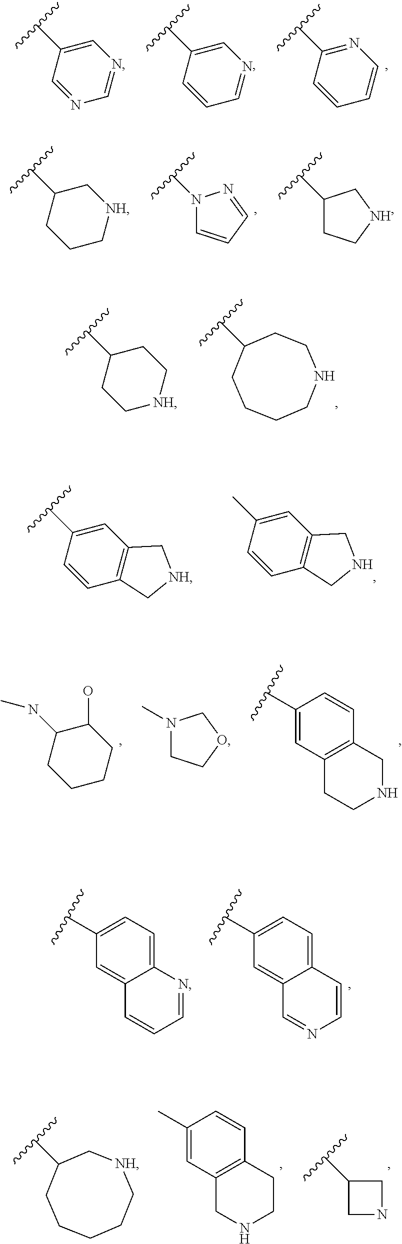

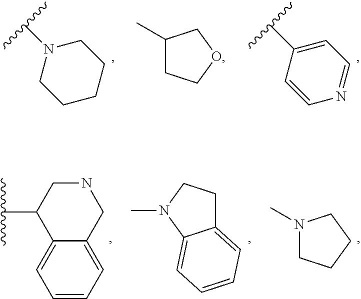

18. A compound selected from Compounds 1-1607 (Table 3), tautomers thereof, deuterated derivatives of the compounds and tautomers, and pharmaceutically acceptable salts of any of the foregoing.

19. A pharmaceutical composition comprising a compound, tautomer, deuterated derivative, or pharmaceutically acceptable salt of any one of claims 1-18 and a pharmaceutically acceptable carrier.

20. The pharmaceutical composition of claim 19, further comprising one or more additional therapeutic agent(s).

21. The pharmaceutical composition of claim 20, wherein the one or more additional therapeutic agent(s) comprise(s) a compound selected from tezacaftor, ivacaftor, deutivacaftor, and pharmaceutically acceptable salts thereof.

22. The pharmaceutical composition of claim 21, wherein the composition comprises tezacaftor and ivacaftor.

23. The pharmaceutical composition of claim 21, wherein the composition comprises tezacaftor and deutivacaftor.

24. A pharmaceutical composition comprising:
(a) at least one compound, tautomer, deuterated derivative, or pharmaceutically acceptable salt according to of any one of claims 1 to 18;
(b) at least one pharmaceutically acceptable carrier; and
optionally one or more of:
(c) (i) a compound chosen from tezacaftor: 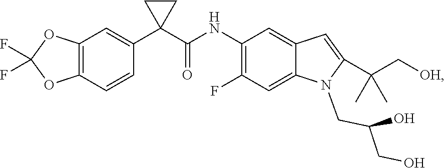 [SMILES: CC(C)(CO)c1cc2cc(NC(=O)C3(c4ccc5c(c4)OC(F)(F)O5)CC3)c(F)cc2n1C[C@@H](O)CO]
and pharmaceutically acceptable salts and deuterated derivatives thereof; and
(ii) a compound chosen from
ivacaftor: 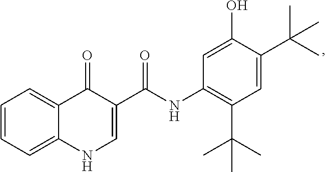 [SMILES: CC(C)(C)c1cc(C(C)(C)C)c(NC(=O)c2c[nH]c3ccccc3c2=O)cc1O]
deutivacaftor: 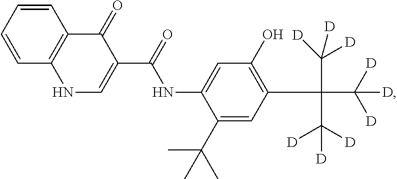
and deuterated derivatives and pharmaceutically acceptable salts thereof.

25. A method of treating cystic fibrosis comprising administering to a patient in need thereof a compound, tautomer, deuterated derivative, or pharmaceutically acceptable salt of any one of claims 1 to 18 or a pharmaceutical composition according to any one of claims 19 to 24.

26. The method of claim 25, further comprising administering to the patient one or more additional therapeutic agent(s) prior to, concurrent with, or subsequent to the compound or the pharmaceutical composition.

27. The method of claim 26, wherein the one or more additional therapeutic agent(s) comprise(s) a compound selected from tezacaftor, ivacaftor, deutivacaftor, lumacaftor, and pharmaceutically acceptable salts thereof.

28. The method of claim 27, wherein the one or more additional therapeutic agent(s) comprise(s) tezacaftor and ivacaftor.

29. The method of claim 27, wherein the one or more additional therapeutic agent(s) comprise(s) ivacaftor and deutivacaftor.

30. The compound, tautomer, deuterated derivative, or pharmaceutically acceptable salt of any one of claims 1 to 18 or the pharmaceutical composition according to any one of claims 19 to 24 for use in the treatment of cystic fibrosis.

31. The compound, tautomer, deuterated derivative, or pharmaceutically acceptable salt of any one of claims 1 to 18 or the pharmaceutical composition according to any one of claims 19 to 24 for use in the manufacture of a medicament for the treatment of cystic fibrosis.

32. A compound selected from Compounds 1-1607, tautomers thereof, deuterated derivatives of those compounds and tautomers, and pharmaceutically acceptable salts of any of the foregoing.

33. A deuterated derivative of a compound selected from Compounds 1-1607.

34. A pharmaceutically acceptable salt of a compound selected from Compounds 1-1607.

35. A compound selected from Compounds 1-1607.

36. A pharmaceutical composition comprising a compound selected from Compounds 1-1607, tautomers thereof, deuterated derivatives of those compounds and tautomers, and pharmaceutically acceptable salts of any of the foregoing and a pharmaceutically acceptable carrier.

37. A pharmaceutical composition comprising a deuterated derivative of a compound selected from Compounds 1-1607 and a pharmaceutically acceptable carrier.

38. A pharmaceutical composition comprising a pharmaceutically acceptable salt of a compound selected from Compounds 1-1607 and a pharmaceutically acceptable carrier.

39. A pharmaceutical composition comprising a compound selected from Compounds 1-1607 and a pharmaceutically acceptable carrier.

40. A pharmaceutical composition comprising (a) a compound selected from Compounds 1-1607, tautomers thereof, deuterated derivatives of those compounds and tautomers, and pharmaceutically acceptable salts of any of the foregoing; (b) a CFTR potentiator; and (c) a pharmaceutically acceptable carrier.

41. A pharmaceutical composition comprising (a) a deuterated derivative of a compound selected from Compounds 1-1607; (b) a CFTR potentiator; and (c) a pharmaceutically acceptable carrier.

42. A pharmaceutical comprising (a) a pharmaceutically acceptable salt of a compound selected from Compounds 1-1607; (b) a CFTR potentiator; and (c) a pharmaceutically acceptable carrier.

43. A pharmaceutical composition comprising (a) a compound selected from Compounds 1-1607; (b) a CFTR potentiator; and (c) a pharmaceutically acceptable carrier.

44. A pharmaceutical composition comprising (a) a compound selected from Compounds 1-1607, tautomers thereof, deuterated derivatives of those compounds and tautomers, and pharmaceutically acceptable salts of any of the foregoing; (b) an additional CFTR corrector; and (c) a pharmaceutically acceptable carrier.

45. A pharmaceutical composition comprising (a) a deuterated derivative of a compound selected from Compounds 1-1607; (b) an additional CFTR corrector; and (c) a pharmaceutically acceptable carrier.

46. A pharmaceutical composition comprising (a) a pharmaceutically acceptable salt of a compound selected from Compounds 1-1607; (b) an additional CFTR corrector; and (c) a pharmaceutically acceptable carrier.

47. A pharmaceutical composition comprising (a) a compound selected from Compounds 1-1607; (b) an additional CFTR corrector; and (c) a pharmaceutically acceptable carrier.

48. A pharmaceutical composition comprising (a) a compound selected from Compounds 1-1607, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier.

49. A pharmaceutical composition comprising (a) a deuterated derivative of a compound selected from Compounds 1-1607; (b) an additional CFTR corrector; (c) a CFTR potentiator; and (d) a pharmaceutically acceptable carrier.

50. A pharmaceutical composition comprising (a) a pharmaceutically acceptable salt of a compound selected from Compounds 1-1607; (b) an additional CFTR corrector; (c) a CFTR potentiator; and (d) a pharmaceutically acceptable carrier.

51. A pharmaceutical composition comprising (a) a compound selected from Compounds 1-1607; (b) an additional CFTR corrector; (c) a CFTR potentiator; and (d) a pharmaceutically acceptable carrier.

52. A compound selected from Compounds 1-1607, tautomers thereof, deuterated derivatives of those compounds and tautomers, and pharmaceutically acceptable salts of any of the foregoing for use in a method of treating cystic fibrosis.

53. A deuterated derivative of a compound selected from Compounds 1-1607 for use in a method of treating cystic fibrosis.

54. A pharmaceutically acceptable salt of a compound selected from Compounds 1-1607 for use in a method of treating cystic fibrosis.

55. A compound selected from Compounds 1-1607 for use in a method of treating cystic fibrosis.

56. A pharmaceutical composition comprising a compound selected from Compounds 1-1607, tautomers thereof, deuterated derivatives of those compounds and tautomers, and pharmaceutically acceptable salts of any of the foregoing and a pharmaceutically acceptable carrier for use in a method of treating cystic fibrosis.

57. A pharmaceutical composition comprising a deuterated derivative of a compound selected from Compounds 1-1607 and a pharmaceutically acceptable carrier for use in a method of treating cystic fibrosis.

58. A pharmaceutical composition comprising a pharmaceutically acceptable salt of a compound selected from Compounds 1-1607 and a pharmaceutically acceptable carrier for use in a method of treating cystic fibrosis.

59. A pharmaceutical composition comprising a compound selected from Compounds 1-1607 and a pharmaceutically acceptable carrier for use in a method of treating cystic fibrosis.

60. A pharmaceutical composition comprising (a) a compound selected from Compounds 1-1607, tautomers thereof, deuterated derivatives of those compounds and tautomers, and pharmaceutically acceptable salts of any of the foregoing; (b) a CFTR potentiator; and (c) a pharmaceutically acceptable carrier for use in a method of treating cystic fibrosis.

61. A pharmaceutical comprising (a) a deuterated derivative of a compound selected from Compounds 1-1607; (b) a CFTR potentiator; and (c) a pharmaceutically acceptable carrier for use in a method of treating cystic fibrosis.

62. A pharmaceutical composition comprising (a) a pharmaceutically acceptable salt of a compound selected from Compounds 1-1607; (b) a CFTR potentiator; and (c) a pharmaceutically acceptable carrier for use in a method of treating cystic fibrosis.

63. A pharmaceutical composition comprising (a) a compound selected from Compounds 1-1607; (b) a CFTR potentiator; and (c) a pharmaceutically acceptable carrier.

64. A pharmaceutical composition comprising (a) a compound selected from Compounds 1-1607, tautomers thereof, deuterated derivatives of those compounds and tautomers, and pharmaceutically acceptable salts of any of the foregoing; (b) an additional CFTR corrector; and (c) a pharmaceutically acceptable carrier for use in a method of treating cystic fibrosis.

65. A pharmaceutical composition comprising (a) a deuterated derivative of a compound selected from Compounds 1-1607; (b) an additional CFTR corrector; and (c) a pharmaceutically acceptable carrier for use in a method of treating cystic fibrosis.

66. A pharmaceutical composition comprising (a) a pharmaceutically acceptable salt of a compound selected from Compounds 1-1607; (b) an additional CFTR corrector; and (c) a pharmaceutically acceptable carrier for use in a method of treating cystic fibrosis.

67. A pharmaceutical composition comprising (a) a compound selected from Compounds 1-1607; (b) an additional CFTR corrector; and (c) a pharmaceutically acceptable carrier for use in a method of treating cystic fibrosis.

68. A pharmaceutical composition comprising (a) a compound selected from Compounds 1-1607, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier for use in a method of treating cystic fibrosis.

69. A pharmaceutical composition comprising (a) a deuterated derivative of a compound selected from Compounds 1-1607; (b) an additional CFTR corrector; (c) a CFTR potentiator; and (d) a pharmaceutically acceptable carrier for use in a method of treating cystic fibrosis.

70. A pharmaceutical composition comprising (a) a pharmaceutically acceptable salt of a compound selected from Compounds 1-1607; (b) an additional CFTR corrector; (c) a CFTR potentiator; and (d) a pharmaceutically acceptable carrier for use in a method of treating cystic fibrosis.

71. A pharmaceutical composition comprising (a) a compound selected from Compounds 1-1607; (b) an additional CFTR corrector; (c) a CFTR potentiator; and (d) a pharmaceutically acceptable carrier for use in a method of treating cystic fibrosis.
